(19) United States
(12) Patent Application Publication
(10) Pub. No.: US 2023/0143612 A1
(43) Pub. Date: May 11, 2023

(54) SUBSTITUTED 1,1'-BIPHENYL COMPOUNDS AND METHODS USING SAME

(71) Applicant: Arbutus Biopharma Corporation, Burnaby, CA (US)

(72) Inventors: Vijay Ahuja, Princeton, NJ (US); Yingzhi Bi, Plainsboro, NJ (US); Andrew G. Cole, Cranbury, NJ (US); Bruce D. Dorsey, Ambler, PA (US); Yi Fan, Doylestown, PA (US); Ramesh Kakarla, San Diego, CA (US); Sharon Marie Kirk, Philadelphia, PA (US); Duyan Nguyen, Ambler, PA (US); Seyma Ozturk, Southampton, PA (US); Jorge G. Quintero, Cherry Hill, NJ (US); Michael J. Sofia, Doylestown, PA (US)

(21) Appl. No.: 17/795,422

(22) PCT Filed: Feb. 1, 2021
(86) PCT No.: PCT/US2021/016068

§ 371 Date: Jul. 26, 2022

Related U.S. Application Data
(60) Provisional application No. 62/969,417, filed on Feb. 3, 2020.
(60) Provisional application No. 63/056,769, filed on Jul. 27, 2020.

(51) Int. Cl. C07D 471/04; A61K 45/06; A61K 39/395; A61K 31/704; A61K 31/7068; A61K 31/519; C07D 401/12; A61K 31/517; A61K 31/5513; A61K 31/444; C07D 401/14; A61K 31/55; C07D 213/85; C07D 241/20; A61K 31/4965; C07D 491/048; A61K 31/4355; A61K 31/497; C07D 519/00; C07D 495/04; A61K 31/4709; A61K 31/4725; C07D 487/04; C07D 413/14; A61K 31/4439; C07D 513/04; A61K 31/5025; A61K 31/553; C07D 498/04; A61K 31/551; A61K 31/53

(52) U.S. Cl. CPC C07D 471/04; A61K 45/06; A61K 39/3955; A61K 31/704; A61K 31/7068; A61K 31/519; C07D 401/12; A61K 31/517; A61K 31/5513; A61K 31/444; C07D 401/14; A61K 31/55; C07D 213/85; C07D 241/20; A61K 31/4965; C07D 491/048; A61K 31/4355; A61K 31/497; C07D 519/00; C07D 495/04; A61K 31/4709; A61K 31/4725; C07D 487/04; C07D 413/14; A61K 31/4439; C07D 513/04; A61K 31/5025; A61K 31/553; C07D 498/04; A61K 31/551; A61K 31/53

(57) ABSTRACT

The present invention includes substituted 1,1′-biphenyl compounds, analogues thereof, and compositions comprising the same. In one aspect, the compounds contemplated in the invention can be used to treat, ameliorate, or prevent hepatitis B virus (HBV) and/or hepatitis D virus (HDV) infections in a patient. In another aspect, the compounds contemplated in the invention can be used to treat, ameliorate, and/or prevent cancer in a patient.

### CROSS-REFERENCE TO RELATED APPLICATIONS

This application claims priority under 35 U.S.C. § 119(e) to U.S. Provisional Application No. 62/969,417, filed Feb. 3, 2020, and U.S. Provisional Application No. 63/056,769, filed Jul. 27, 2020, the contents of each of which are incorporated by reference herein in their entireties.

### BACKGROUND OF THE INVENTION

Hepatitis B virus (HBV) is a noncytopathic, liver tropic DNA virus belonging to Hepadnaviridae family. HBV infection is one of the world's most prevalent diseases, being listed by National Institute of Allergy and Infectious Diseases (NIAID) as a High Priority Area of Interest. Although most individuals resolve the infection following acute symptoms, approximately 30% of cases become chronic. 350-400 million people worldwide are estimated to have chronic hepatitis B, leading to 0.5-1 million deaths per year, due largely to the development of hepatocellular carcinoma, cirrhosis and/or other complications.

A limited number of drugs are currently approved for the management of chronic hepatitis B, including two formulations of alpha-interferon (standard and pegylated) and five nucleoside/nucleotide analogues (lamivudine, adefovir, entecavir, telbivudine, and tenofovir) that inhibit HBV DNA polymerase. At present, the first-line treatment choices are entecavir, tenofovir and/or peg-interferon alfa-2a. However, peg-interferon alfa-2a achieves desirable serological milestones in only one third of treated patients, and is frequently associated with severe side effects. Entecavir and tenofovir are potent HBV inhibitors, but require long-term or possibly lifetime administration to continuously suppress HBV replication, and may eventually fail due to emergence of drug-resistant viruses. There is thus a pressing need for the introduction of novel, safe and effective therapies for chronic hepatitis B.

Hepatitis D virus (HDV) is a small circular enveloped RNA virus that can propagate only in the presence of HBV. In particular, HDV requires the HBV surface antigen protein to propagate itself. Infection with both HBV and HDV results in more severe complications compared to infection with HBV alone. These complications include a greater likelihood of experiencing liver failure in acute infections and a rapid progression to liver cirrhosis, with an increased chance of developing liver cancer in chronic infections. In combination with hepatitis B virus, hepatitis D has the highest mortality rate of all the hepatitis infections. The routes of transmission of HDV are similar to those for HBV. Infection is largely restricted to persons at high risk of HBV infection, particularly injecting drug users and persons receiving clotting factor concentrates.

Currently, there is no effective antiviral therapy available for the treatment of acute or chronic type D hepatitis. Interferon-alfa, given weekly for 12 to 18 months, is the only licensed treatment for hepatitis D. Response to this therapy is limited—in only about one-quarter of patients is serum HDV RNA undetectable 6 months post therapy.

There is a need in the art for the identification of novel compounds that can be used to treat, ameliorate, and/or prevent HBV infection in a subject. In certain embodiments, the novel compounds can be used in patients that are HBV infected, patients who are at risk of becoming HBV infected, and/or patients that are infected with drug-resistant HBV. In other embodiments, the HBV-infected subject is further HDV-infected. The present invention addresses this need.

### BRIEF SUMMARY OF THE INVENTION

The invention provides certain compounds of formula (Ia) or (Ib), as defined elsewhere herein, or a salt, solvate, geometric isomer, stereoisomer, tautomer and any mixtures thereof, wherein the substituents in (Ia) or (Ib) are defined elsewhere herein.

The invention further provides pharmaceutical compositions comprising at least one compound of the invention. In certain embodiments, the pharmaceutical compositions further comprise at least one pharmaceutically acceptable carrier. In other embodiments, the pharmaceutical compositions further comprise at least one additional agent that treats, ameliorates, or prevents hepatitis virus infection. In yet other embodiments, the hepatitis virus is hepatitis B virus (HBV). In yet other embodiments, the hepatitis virus is hepatitis D virus (HDV).

The invention further provides a method of treating, ameliorating, and/or preventing hepatitis virus infection in a subject. In certain embodiments, the method comprises administering to the subject a therapeutically effective amount of a compound of the invention, or a salt, solvate, prodrug, stereoisomer, tautomer, or any mixtures thereof. In other embodiments, the subject is infected with HBV. In yet other embodiments, the subject is infected with HDV. In yet other embodiments, the subject is infected with HBV and HDV. In yet other embodiments, the subject is further administered at least one additional agent useful for treating the hepatitis virus infection. In yet other embodiments, the subject is in need of the treatment, amelioration, or prevention of the HBV and/or HDV infection.

The invention further provides a method of treating, ameliorating, and/or preventing cancer in a subject. In certain embodiments, the method comprises administering to the subject a therapeutically effective amount of a compound of the invention, or a salt, solvate, prodrug, stereoisomer, tautomer, or any mixtures thereof. In other embodiments, the subject is further administered at least one additional agent useful for treating, ameliorating, or preventing the cancer. In yet other embodiments, the subject is in need of the treatment, amelioration, and/or prevention of the cancer.

### DETAILED DESCRIPTION OF THE INVENTION

The invention relates, in certain aspects, to the discovery of certain substituted 1,1′-biphenyl compounds. In one aspect, the compounds of the invention are useful to treat, ameliorate and/or prevent hepatitis B virus (HBV) infection and/or hepatitis B virus-hepatitis D virus (HBV-HDV) infection and related conditions in a subject. In certain embodiments, these compounds are administered along with at least one additional agent useful for treating, ameliorating, and/or preventing the viral infection. In other embodiments, the subject is infected with HBV. In yet other embodiments, the HBV-infected subject is further infected with HDV. In another aspect, the compounds of the invention are useful to treat, ameliorate and/or prevent cancer and related conditions in a subject.

### Definitions

As used herein, each of the following terms has the meaning associated with it in this section. Unless defined otherwise, all technical and scientific terms used herein generally have the same meaning as commonly understood by one of ordinary skill in the art to which this invention belongs. Generally, the nomenclature used herein and the laboratory procedures in animal pharmacology, pharmaceutical science, separation science and organic chemistry are those well-known and commonly employed in the art. It should be understood that the order of steps or order for performing certain actions is immaterial, so long as the present teachings remain operable. Moreover, two or more steps or actions can be conducted simultaneously or not.

As used herein, the articles “a” and “an” refer to one or to more than one (i.e., to at least one) of the grammatical object of the article. By way of example, “an element” means one element or more than one element.

As used herein, the term “alkenyl,” employed alone or in combination with other terms, means, unless otherwise stated, a stable monounsaturated or diunsaturated straight chain or branched chain hydrocarbon group having the stated number of carbon atoms. Examples include vinyl, propenyl (or allyl), crotyl, isopentenyl, butadienyl, 1,3-pentadienyl, 1,4-pentadienyl, and the higher homologs and isomers. A functional group representing an alkene is exemplified by —CH₂—CH═CH₂.

As used herein, the term “alkoxy” employed alone or in combination with other terms means, unless otherwise stated, an alkyl group having the designated number of carbon atoms, as defined elsewhere herein, connected to the rest of the molecule via an oxygen atom, such as, for example, methoxy, ethoxy, 1-propoxy, 2-propoxy (or isopropoxy) and the higher homologs and isomers. A specific example is (C₁-C₃)alkoxy, such as, but not limited to, ethoxy and methoxy.

As used herein, the term “alkyl” by itself or as part of another substituent means, unless otherwise stated, a straight or branched chain hydrocarbon having the number of carbon atoms designated (i.e., C₁-C₁₀ means one to ten carbon atoms) and includes straight, branched chain, or cyclic substituent groups. Examples include methyl, ethyl, propyl, isopropyl, butyl, isobutyl, tert-butyl, pentyl, neopentyl, hexyl, and cyclopropylmethyl. A specific embodiment is (C₁-C₆) alkyl, such as, but not limited to, ethyl, methyl, isopropyl, isobutyl, n-pentyl, n-hexyl and cyclopropylmethyl.

As used herein, the term “alkynyl” employed alone or in combination with other terms means, unless otherwise stated, a stable straight chain or branched chain hydrocarbon group with a triple carbon-carbon bond, having the stated number of carbon atoms. Non-limiting examples include ethynyl and propynyl, and the higher homologs and isomers. The term “propargylic” refers to a group exemplified by —CH₂—C≡CH. The term “homopropargylic” refers to a group exemplified by —CH₂CH₂—C≡CH.

As used herein, the term “aromatic” refers to a carbocycle or heterocycle with one or more polyunsaturated rings and having aromatic character, i.e., having (4n+2) delocalized 7c (pi) electrons, where ‘n’ is an integer.

As used herein, the term “aryl” employed alone or in combination with other terms means, unless otherwise stated, a carbocyclic aromatic system containing one or more rings (typically one, two or three rings) wherein such rings may be attached together in a pendent manner, such as a biphenyl, or may be fused, such as naphthalene. Examples include phenyl, anthracyl and naphthyl. Aryl groups also include, for example, phenyl or naphthyl rings fused with one or more saturated or partially saturated carbon rings (e.g., bicyclo[4.2.0]octa-1,3,5-trienyl, or indanyl), which can be substituted at one or more carbon atoms of the aromatic and/or saturated or partially saturated rings.

As used herein, the term “aryl-(C₁-C₆)alkyl” refers to a functional group wherein a one to six carbon alkylene chain is attached to an aryl group, e.g., —CH₂CH₂-phenyl or —CH₂-phenyl (or benzyl). Specific examples are aryl-CH₂— and aryl-CH(CH₃)—. The term “substituted aryl-(C₁-C₆)alkyl” refers to an aryl-(C₁-C₆)alkyl functional group in which the aryl group is substituted. A specific example is substituted aryl(CH₂)—. Similarly, the term “heteroaryl-(C₁-C₆)alkyl” refers to a functional group wherein a one to three carbon alkylene chain is attached to a heteroaryl group, e.g., —CH₂CH₂-pyridyl. A specific example is heteroaryl-(CH₂)—. The term “substituted heteroaryl-(C₁-C₆)alkyl” refers to a heteroaryl-(C₁-C₆)alkyl functional group in which the heteroaryl group is substituted. A specific example is substituted heteroaryl-(CH₂)—.

In one aspect, the terms “co-administered” and “co-administration” as relating to a subject refer to administering to the subject a compound and/or composition of the invention along with a compound and/or composition that may also treat or prevent a disease or disorder contemplated herein. In certain embodiments, the co-administered compounds and/or compositions are administered separately, or in any kind of combination as part of a single therapeutic approach. The co-administered compound and/or composition may be formulated in any kind of combinations as mixtures of solids and liquids under a variety of solid, gel, and liquid formulations, and as a solution.

As used herein, the term “cycloalkyl” by itself or as part of another substituent refers to, unless otherwise stated, a cyclic chain hydrocarbon having the number of carbon atoms designated (i.e., C₃-C₆ refers to a cyclic group comprising a ring group consisting of three to six carbon atoms) and includes straight, branched chain or cyclic substituent groups.

Examples of (C₃-C₆)cycloalkyl groups are cyclopropyl, cyclobutyl, cyclopentyl and cyclohexyl. Cycloalkyl rings can be optionally substituted. Non-limiting examples of cycloalkyl groups include: cyclopropyl, 2-methyl-cyclopropyl, cyclopropenyl, cyclobutyl, 2,3-dihydroxycyclobutyl, cyclobutenyl, cyclopentyl, cyclopentenyl, cyclopentadienyl, cyclohexyl, cyclohexenyl, cycloheptyl, cyclooctanyl, decalinyl, 2,5-dimethylcyclopentyl, 3,5-dichlorocyclohexyl, 4-hydroxycyclohexyl, 3,3,5-trimethylcyclohex-1-yl, octahydropentalenyl, octahydro-1H-indenyl, 3a,4,5,6,7,7a-hexahydro-3H-inden-4-yl, decahydroazulenyl; bicyclo[6.2.0]decanyl, decahydronaphthalenyl, and dodecahydro-1H-fluorenyl. The term “cycloalkyl” also includes bicyclic hydrocarbon rings, non-limiting examples of which include, bicyclo-[2.1.1]hexanyl, bicyclo[2.2.1]heptanyl, bicyclo[3.1.1]heptanyl, 1,3-dimethyl[2.2.1] heptan-2-yl, bicyclo[2.2.2]octanyl, and bicyclo[3.3.3]undecanyl.

As used herein, the term “DCM” refers to dichloromethane.

As used herein, a “disease” is a state of health of a subject wherein the subject cannot maintain homeostasis, and wherein if the disease is not ameliorated then the subject's health continues to deteriorate.

As used herein, a “disorder” in a subject is a state of health in which the subject is able to maintain homeostasis, but in which the subject's state of health is less favorable than it would be in the absence of the disorder. Left untreated, a disorder does not necessarily cause a further decrease in the subject's state of health.

As used herein, the term “halide” refers to a halogen atom bearing a negative charge. The halide anions are fluoride (F⁻), chloride (Cl⁻), bromide (Br⁻), and iodide (I⁻).

As used herein, the term “halo” or “halogen” alone or as part of another substituent refers to, unless otherwise stated, a fluorine, chlorine, bromine, or iodine atom.

As used herein, the term “Hepatitis B virus” (or HBV) refers to a virus species of the genus Orthohepadnavirus, which is a part of the Hepadnaviridae family of viruses, and that is capable of causing liver inflammation in humans.

As used herein, the term “Hepatitis D virus” (or HDV) refers to a virus species of the genus Deltaviridae, which is capable of causing liver inflammation in humans. The HDV particle comprises an envelope, which is provided by HBV and surrounds the RNA genome and the HDV antigen. The HDV genome is a single, negative stranded, circular RNA molecule nearly 1.7 kb in length. The genome contains several sense and antisense open reading frames (ORFs), only one of which is functional and conserved. The RNA genome is replicated through an RNA intermediate, the antigenome. The genomic RNA and its complement, the antigenome, can function as ribozymes to carry out self-cleavage and self-ligation reactions. A third RNA present in the infected cell, also complementary to the genome, but 800 bp long and polyadenylated, is the mRNA for the synthesis of the delta antigen (HDAg).

As used herein, the term “heteroalkenyl” by itself or in combination with another term refers to, unless otherwise stated, a stable straight or branched chain monounsaturated or diunsaturated hydrocarbon group consisting of the stated number of carbon atoms and one or two heteroatoms selected from the group consisting of O, N, and S, and wherein the nitrogen and sulfur atoms may optionally be oxidized and the nitrogen heteroatom may optionally be quaternized. Up to two heteroatoms may be placed consecutively. Examples include —CH═CH—O—CH₃, —CH═CH—CH₂—OH, —CH₂—CH═N—OCH₃, —CH═CH—N(CH₃)—CH₃, and —CH₂—CH═CH—CH₂—SH.

As used herein, the term “heteroalkyl” by itself or in combination with another term refers to, unless otherwise stated, a stable straight or branched chain alkyl group consisting of the stated number of carbon atoms and one or two heteroatoms selected from the group consisting of O, N, and S, and wherein the nitrogen and sulfur atoms may be optionally oxidized and the nitrogen heteroatom may be optionally quaternized. The heteroatom(s) may be placed at any position of the heteroalkyl group, including between the rest of the heteroalkyl group and the fragment to which it is attached, as well as attached to the most distal carbon atom in the heteroalkyl group. Examples include: —OCH₂CH₂CH₃, —CH₂CH₂CH₂OH, —CH₂CH₂NHCH₃, —CH₂SCH₂CH₃, and —CH₂CH₂S(═O)CH₃. Up to two heteroatoms may be consecutive, such as, for example, —CH₂NH—OCH₃, or —CH₂CH₂SSCH₃.

As used herein, the term “heteroaryl” or “heteroaromatic” refers to a heterocycle having aromatic character. A polycyclic heteroaryl may include one or more rings that are partially saturated. Examples include tetrahydroquinoline and 2,3-dihydrobenzofuryl.

As used herein, the term “heterocycle” or “heterocyclyl” or “heterocyclic” by itself or as part of another substituent refers to, unless otherwise stated, an unsubstituted or substituted, stable, mono- or multi-cyclic heterocyclic ring system that comprises carbon atoms and at least one heteroatom selected from the group consisting of N, O, and S, and wherein the nitrogen and sulfur heteroatoms may be optionally oxidized, and the nitrogen atom may be optionally quaternized. The heterocyclic system may be attached, unless otherwise stated, at any heteroatom or carbon atom that affords a stable structure. A heterocycle may be aromatic or non-aromatic in nature. In certain embodiments, the heterocycle is a heteroaryl.

Examples of non-aromatic heterocycles include monocyclic groups such as aziridine, oxirane, thiirane, azetidine, oxetane, thietane, pyrrolidine, pyrroline, imidazoline, pyrazolidine, dioxolane, sulfolane, 2,3-dihydrofuran, 2,5-dihydrofuran, tetrahydrofuran, thiophane, piperidine, 1,2,3,6-tetrahydropyridine, 1,4-dihydropyridine, piperazine, morpholine, thiomorpholine, pyran, 2,3-dihydropyran, tetrahydropyran, 1,4-dioxane, 1,3-dioxane, homopiperazine, homopiperidine, 1,3-dioxepane, 4,7-dihydro-1,3-dioxepin and hexamethyleneoxide.

Examples of heteroaryl groups include pyridyl, pyrazinyl, pyrimidinyl (such as, but not limited to, 2- and 4-pyrimidinyl), pyridazinyl, thienyl, furyl, pyrrolyl, imidazolyl, thiazolyl, oxazolyl, pyrazolyl, isothiazolyl, 1,2,3-triazolyl, 1,2,4-triazolyl, 1,3,4-triazolyl, tetrazolyl, 1,2,3-thiadiazolyl, 1,2,3-oxadiazolyl, 1,3,4-thiadiazolyl and 1,3,4-oxadiazolyl.

Examples of polycyclic heterocycles include indolyl (such as, but not limited to, 3-, 4-5-, 6- and 7-indolyl), indolinyl, quinolyl, tetrahydroquinolyl, isoquinolyl (such as, but not limited to, 1- and 5-isoquinolyl), 1,2,3,4-tetrahydroisoquinolyl, cinnolinyl, quinoxalinyl (such as, but not limited to, 2- and 5-quinoxalinyl), quinazolinyl, phthalazinyl, 1,8-naphthyridinyl, 1,4-benzodioxanyl, coumarin, dihydrocoumarin, 1,5-naphthyridinyl, benzofuryl (such as, but not limited to, 3-, 4-, 5-, 6- and 7-benzofuryl), 2,3-dihydrobenzofuryl, 1,2-benzisoxazolyl, benzothienyl (such as, but not limited to, 3-, 4-, 5-, 6-, and 7-benzothienyl), benzoxazolyl, benzothiazolyl (such as, but not limited to, 2-benzothiazolyl and 5-benzothiazolyl), purinyl, benzimidazolyl, benztriazolyl, thioxanthinyl, carbazolyl, carbolinyl, acridinyl, pyrrolizidinyl, and quinolizidinyl.

The aforementioned listing of heterocyclyl and heteroaryl moieties is intended to be representative and not limiting.

As described herein, the term “PD-L1 inhibitor” includes any compound that is capable of inhibiting the expression and/or function of the protein Programmed Death-Ligand 1 (PD-L1) either directly or indirectly. PD-L1, also known as cluster of differentiation 274 (CD274) or B7 homolog 1 (B7-H1), is a type 1 transmembrane protein that plays a major role in suppressing the adaptive arm of immune system during pregnancy, tissue allograft transplants, autoimmune disease, and hepatitis. PD-L1 binds to its receptor, the inhibitory checkpoint molecule PD-1 (which is found on activated T cells, B cells, and myeloid cells) so as to modulate activation or inhibition of the adaptive arm of immune system. In certain embodiments, the PD-L1 inhibitor inhibits the expression and/or function of PD-L1 by at least 5%, at least 10%, at least 20%, at least 50%, at least 75%, or at least 90%.

As used herein, the term “pharmaceutical composition” or “composition” refers to a mixture of at least one compound useful within the invention with a pharmaceutically acceptable carrier. The pharmaceutical composition facilitates administration of the compound to a subject.

As used herein, the term “pharmaceutically acceptable” refers to a material, such as a carrier or diluent, which does not abrogate the biological activity or properties of the compound useful within the invention, and is relatively non-toxic, i.e., the material may be administered to a subject without causing undesirable biological effects or interacting in a deleterious manner with any of the components of the composition in which it is contained.

As used herein, the term “pharmaceutically acceptable carrier” means a pharmaceutically acceptable material, composition or carrier, such as a liquid or solid filler, stabilizer, dispersing agent, suspending agent, diluent, excipient, thickening agent, solvent or encapsulating material, involved in carrying or transporting a compound useful within the invention within or to the subject such that it may perform its intended function. Typically, such constructs are carried or transported from one organ, or portion of the body, to another organ, or portion of the body. Each carrier must be “acceptable” in the sense of being compatible with the other ingredients of the formulation, including the compound useful within the invention, and not injurious to the subject. Some examples of materials that may serve as pharmaceutically acceptable carriers include: sugars, such as lactose, glucose and sucrose; starches, such as corn starch and potato starch; cellulose, and its derivatives, such as sodium carboxymethyl cellulose, ethyl cellulose and cellulose acetate; powdered tragacanth; malt; gelatin; talc; excipients, such as cocoa butter and suppository waxes; oils, such as peanut oil, cottonseed oil, safflower oil, sesame oil, olive oil, corn oil and soybean oil; glycols, such as propylene glycol; polyols, such as glycerin, sorbitol, mannitol and polyethylene glycol; esters, such as ethyl oleate and ethyl laurate; agar; buffering agents, such as magnesium hydroxide and aluminum hydroxide; surface active agents; alginic acid; pyrogen-free water; isotonic saline; Ringer's solution; ethyl alcohol; phosphate buffer solutions; and other non-toxic compatible substances employed in pharmaceutical formulations. As used herein, “pharmaceutically acceptable carrier” also includes any and all coatings, antibacterial and antifungal agents, and absorption delaying agents, and the like that are compatible with the activity of the compound useful within the invention, and are physiologically acceptable to the subject. Supplementary active compounds may also be incorporated into the compositions. The “pharmaceutically acceptable carrier” may further include a pharmaceutically acceptable salt of the compound useful within the invention.

Other additional ingredients that may be included in the pharmaceutical compositions used in the practice of the invention are known in the art and described, for example in Remington's Pharmaceutical Sciences (Genaro, Ed., Mack Publishing Co., 1985, Easton, Pa.), which is incorporated herein by reference.

As used herein, the language “pharmaceutically acceptable salt” refers to a salt of the administered compound prepared from pharmaceutically acceptable non-toxic acids and/or bases, including inorganic acids, inorganic bases, organic acids, inorganic bases, solvates (including hydrates) and clathrates thereof.

As used herein, a “pharmaceutically effective amount,” “therapeutically effective amount,” or “effective amount” of a compound is that amount of compound that is sufficient to provide a beneficial effect to the subject to which the compound is administered.

The term “prevent,” “preventing,” or “prevention” as used herein means avoiding or delaying the onset of symptoms associated with a disease or condition in a subject that has not developed such symptoms at the time the administering of an agent or compound commences. Disease, condition and disorder are used interchangeably herein.

By the term “specifically bind” or “specifically binds” as used herein is meant that a first molecule preferentially binds to a second molecule (e.g., a particular receptor or enzyme), but does not necessarily bind only to that second molecule.

As used herein, the terms “subject” and “individual” and “patient” can be used interchangeably and may refer to a human or non-human mammal or a bird. Non-human mammals include, for example, livestock and pets, such as ovine, bovine, porcine, canine, feline and murine mammals. In certain embodiments, the subject is human.

As used herein, the term “substituted” refers to that an atom or group of atoms has replaced hydrogen as the substituent attached to another group.

As used herein, the term “substituted alkyl,” “substituted cycloalkyl,” “substituted alkenyl,” or “substituted alkynyl” refers to alkyl, cycloalkyl, alkenyl or alkynyl, as defined elsewhere herein, substituted by one, two or three substituents independently selected from the group consisting of halogen, —OH, alkoxy, tetrahydro-2-H-pyranyl, —NH₂, —NH(C₁-C₆ alkyl), —N(C₁-C₆ alkyl)₂, 1-methyl-imidazol-2-yl, pyridin-2-yl, pyridin-3-yl, pyridin-4-yl, —C(═O)OH, —C(═O)O(C₁-C₆)alkyl, trifluoromethyl, —C—N, —C(═O)NH₂, —C(═O)NH(C₁-C₆)alkyl, —C(═O)N((C₁-C₆)alkyl)₂, —SO₂NH₂, —SO₂NH(C₁-C₆ alkyl), —SO₂N(C₁-C₆ alkyl)₂, —C(═NH)NH₂, and —NO₂, in certain embodiments containing one or two substituents independently selected from halogen, —OH, alkoxy, —NH₂, trifluoromethyl, —N(CH₃)₂, and —C(═O)OH, in certain embodiments independently selected from halogen, alkoxy and —OH.

Examples of substituted alkyls include, but are not limited to, 2,2-difluoropropyl, 2-carboxycyclopentyl and 3-chloropropyl.

For aryl, aryl-(C₁-C₃)alkyl and heterocyclyl groups, the term “substituted” as applied to the rings of these groups refers to any level of substitution, namely mono—, di—, tri—, tetra—, or penta-substitution, where such substitution is permitted. The substituents are independently selected, and substitution may be at any chemically accessible position. In certain embodiments, the substituents vary in number between one and four. In other embodiments, the substituents vary in number between one and three. In yet another embodiments, the substituents vary in number between one and two. In yet other embodiments, the substituents are independently selected from the group consisting of C₁-C₆ alkyl, —OH, C₁-C₆ alkoxy, halo, amino, acetamido and nitro. As used herein, where a substituent is an alkyl or alkoxy group, the carbon chain may be branched, straight or cyclic.

In certain embodiments, each occurrence of alkyl or cycloalkyl is independently optionally substituted with at least one substituent selected from the group consisting of C₁-C₆ alkyl, halo, —OR, phenyl (thus yielding, in non-limiting examples, optionally substituted phenyl-(C₁-C₃ alkyl), such as, but not limited to, benzyl or substituted benzyl) and —N(R)(R), wherein each occurrence of R is independently H, C₁-C₆ alkyl or C₃-C₈ cycloalkyl. In other embodiments, each occurrence of aryl or heteroaryl is independently optionally substituted with at least one substituent selected from the group consisting of C₁-C₆ alkyl, C₁-C₆ haloalkyl, C₁-C₆ haloalkoxy, halo, —CN, —OR, —N(R)(R), —NO₂, —S(═O)₂N(R)(R), acyl, and C₁-C₆ alkoxycarbonyl, wherein each occurrence of R is independently H, C₁-C₆ alkyl or C₃-C₈ cycloalkyl. In yet other embodiments, each occurrence of aryl or heteroaryl is independently optionally substituted with at least one substituent selected from the group consisting of C₁-C₆ alkyl, C₁-C₆ haloalkyl, C₁-C₆ haloalkoxy, halo, —CN, —OR, —N(R)(R), and C₁-C₆ alkoxycarbonyl, wherein each occurrence of R is independently H, C₁-C₆ alkyl or C₃-C₈ cycloalkyl.

Unless otherwise noted, when two substituents are taken together to form a ring having a specified number of ring atoms (e.g., R² and R³ taken together with the nitrogen to which they are attached to form a ring having from 3 to 7 ring members), the ring can have carbon atoms and optionally one or more (e.g., 1 to 3) additional heteroatoms independently selected from nitrogen, oxygen, or sulfur. The ring can be saturated or partially saturated, and can be optionally substituted.

Whenever a term or either of their prefix roots appear in a name of a substituent the name is to be interpreted as including those limitations provided herein. For example, whenever the term “alkyl” or “aryl” or either of their prefix roots appear in a name of a substituent (e.g., arylalkyl, alkylamino) the name is to be interpreted as including those limitations given elsewhere herein for “alkyl” and “aryl” respectively.

In certain embodiments, substituents of compounds are disclosed in groups or in ranges. It is specifically intended that the description include each and every individual subcombination of the members of such groups and ranges. For example, the term “C₁₋₆ alkyl” is specifically intended to individually disclose C₁, C₂, C₃, C₄, C₅, C₆, C₁-C₆, C₁-C₅, C₁-C₄, C₁-C₃, C₁-C₂, C₂-C₆, C₂-C₅, C₂-C₄, C₂-C₃, C₃-C₆, C₃-C₈, C₃-C₄, C₄-C₆, C₄-C₅, and C₅-C₆ alkyl.

The terms “treat,” “treating,” and “treatment,” as used herein, means reducing the frequency or severity with which symptoms of a disease or condition are experienced by a subject by virtue of administering an agent or compound to the subject.

Ranges: throughout this disclosure, various aspects of the invention can be presented in a range format. It should be understood that the description in range format is merely for convenience and brevity and should not be construed as an inflexible limitation on the scope of the invention. Accordingly, the description of a range should be considered to have specifically disclosed all the possible sub-ranges as well as individual numerical values within that range. For example, description of a range such as from 1 to 6 should be considered to have specifically disclosed sub-ranges such as from 1 to 3, from 1 to 4, from 1 to 5, from 2 to 4, from 2 to 6, from 3 to 6 etc., as well as individual and partial numbers within that range, for example, 1, 2, 2.7, 3, 4, 5, 5.3, and 6. This applies regardless of the breadth of the range.

### Compounds

Programmed death-ligand 1 (PD-L1), which is also known as cluster of differentiation 274 (CD274) or B7 homolog 1 (B7-H1), is a human transmembrane protein that plays a major role in suppressing the immune system as needed. Generally, the presence of a foreign antigen in the body triggers proliferation of antigen-specific CD8+ T cells in the lymph nodes. However, binding of PD-L1 to the receptor programmed cell death protein 1 (PD-1) or B7.1 membrane protein (both of which are found on activated T cells, B cells, and myeloid cells), transmits an inhibitory signal, which reduces proliferation of the CD8+ T cells in the lymph nodes. Such interaction effectively suppresses the immune response and avoids detection and destruction of the antigens.

In certain embodiments, small-molecule immunomodulators targeting the PD-1/PD-L1 signaling pathway are used to treat, ameliorate, and/or prevent hepatitis B virus (HBV) infection and related conditions in a subject. In other embodiments, inhibition of PDL-1 enhances the immune response to at least one HBV antigen.

The invention include a compound of formula (Ia) or (Ib), or a salt, solvate, prodrug, stereoisomer (such as, in a non-limiting example, an enantiomer or diastereoisomer, and any mixtures thereof, such as, in a non-limiting example, mixtures in any proportion of enantiomers and/or diastereoisomers thereof), tautomer, and/or geometric isomer, and any mixtures thereof. It should be noted that the absolute stereochemistry of the chiral center(s) represented in any structure depicted herein and/or compound named herein is merely illustrative and non-limiting.

In certain embodiments, the compound of formula (Ia) is:

wherein:

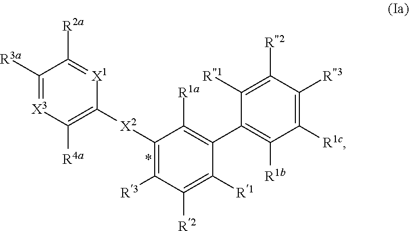

X¹ is selected from the group consisting of CR^{X1} and N, wherein each occurrence of R^{X1} is independently selected from the group consisting of H, C₁-C₃ alkyl, C₃-C₈ cycloalkyl, C₁-C₃ alkoxy, cyano, halogen, C₁-C₃ haloalkyl, and C₁-C₃ haloalkoxy;

X² is selected from the group consisting of bond, —OCH₂—**, —CH₂O—**, —CH═CH—**, —CF═CH—**, —CH═CF—**, —CF═CF—**, —C(═O)NH—**, and —NHC(═O)—**,

wherein the bond marked with ** is to the phenyl ring carbon marked with *;

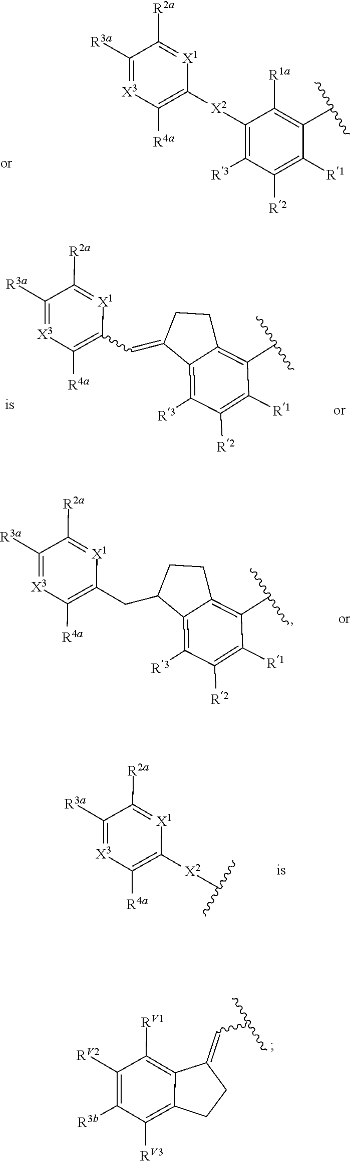

X³ is selected from the group consisting of CR^{X3} and N, wherein each occurrence of R^{X3} is independently selected from the group consisting of H, C₁-C₃ alkyl, C₃-C₈ cycloalkyl, C₁-C₃ alkoxy, cyano, halogen, C₁-C₃ haloalkyl, and C₁-C₃ haloalkoxy;

R′¹, R′², and R′³ are independently selected from the group consisting of H, C₁-C₃ alkyl, C₃-C₈ cycloalkyl, C₁-C₃ alkoxy, cyano, halogen, C₁-C₃ haloalkyl, and C₁-C₃ haloalkoxy;

R″¹, R″², and R″³ are independently selected from the group consisting of H, C₁-C₃ alkyl, C₃-C₈ cycloalkyl, C₁-C₃ alkoxy, cyano, halogen, C₁-C₃ haloalkyl, and C₁-C₃ haloalkoxy;

R^{1a} is selected from the group consisting of H, C₁-C₃ alkyl, C₃-C₈ cycloalkyl, C₁-C₃ alkoxy, cyano, halogen, C₁-C₃ haloalkyl, and C₁-C₃ haloalkoxy;

R^{1b} is selected from the group consisting of H, C₁-C₃ alkyl, C₃-C₈ cycloalkyl, C₁-C₃ alkoxy, cyano, halogen, C₁-C₃ haloalkyl, and C₁-C₃ haloalkoxy;

R^{1c} is selected from the group consisting of:

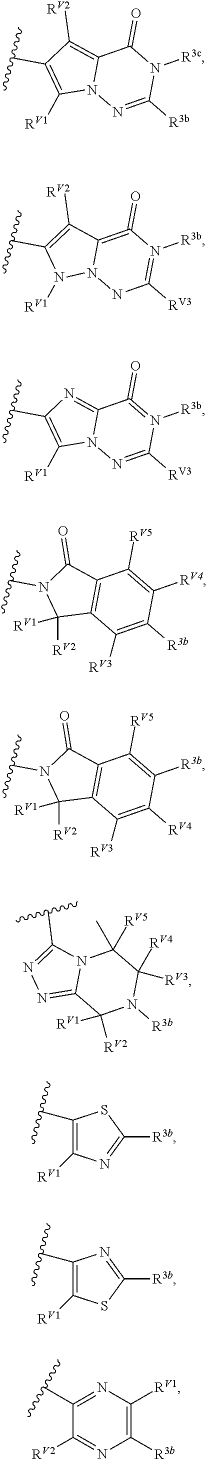

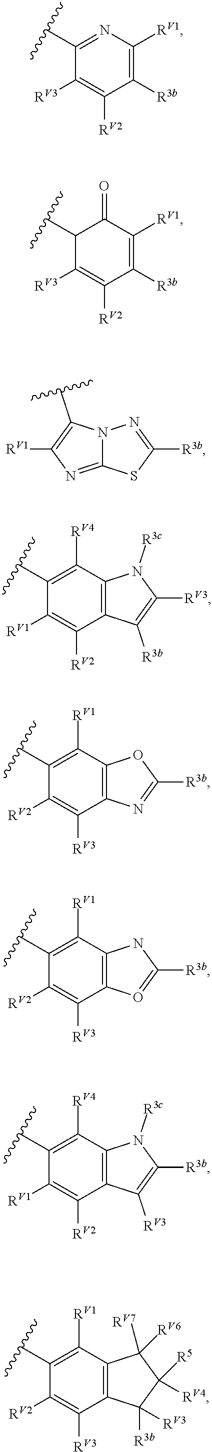

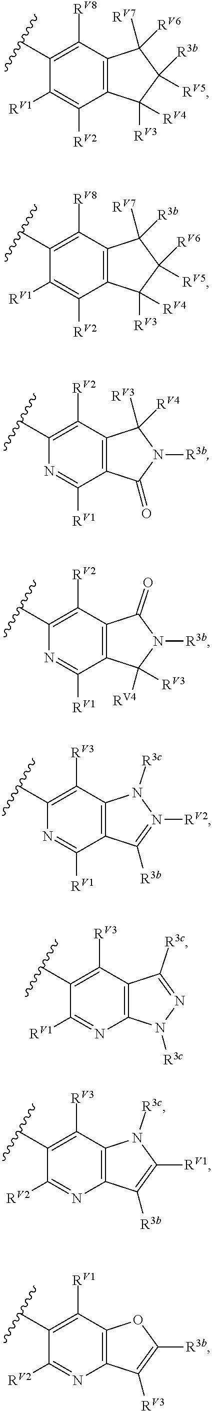

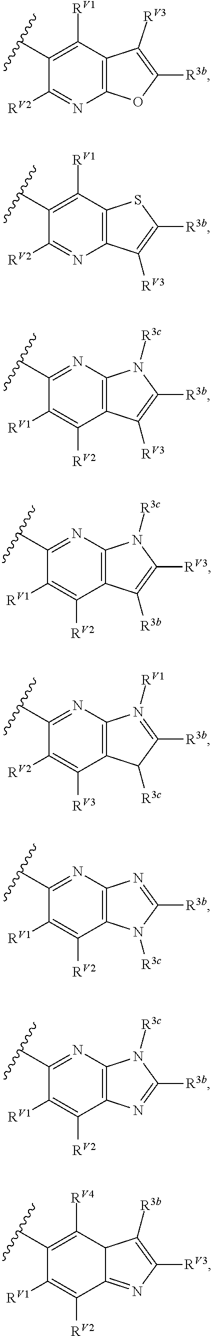

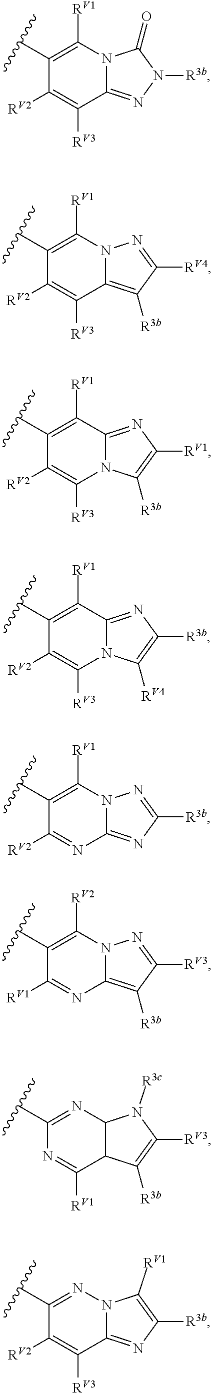

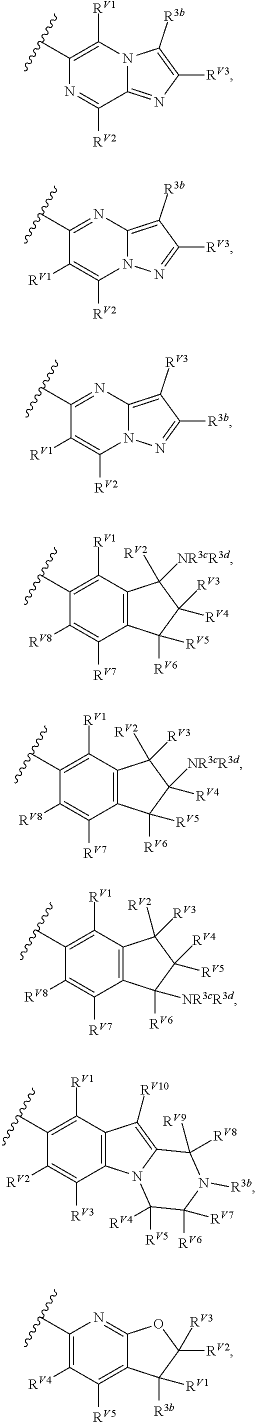

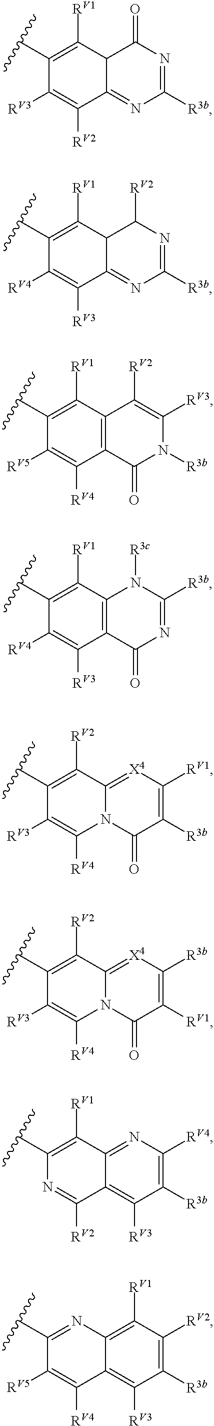

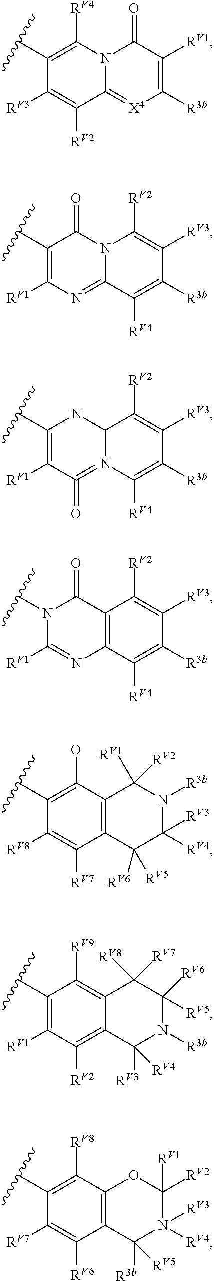

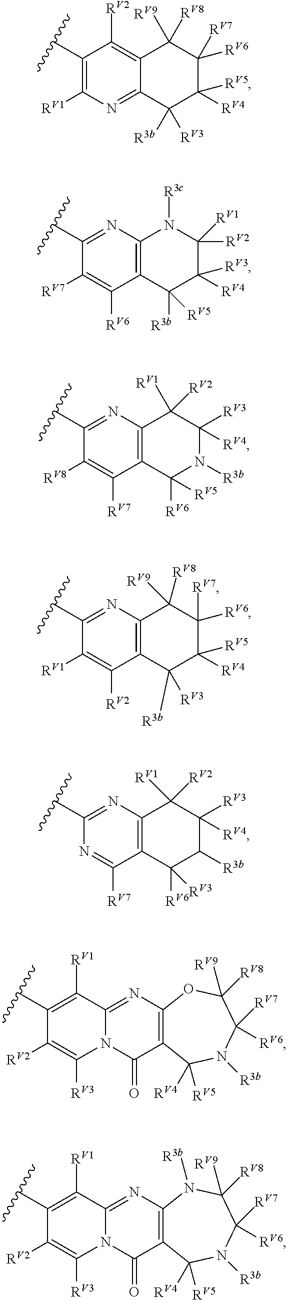

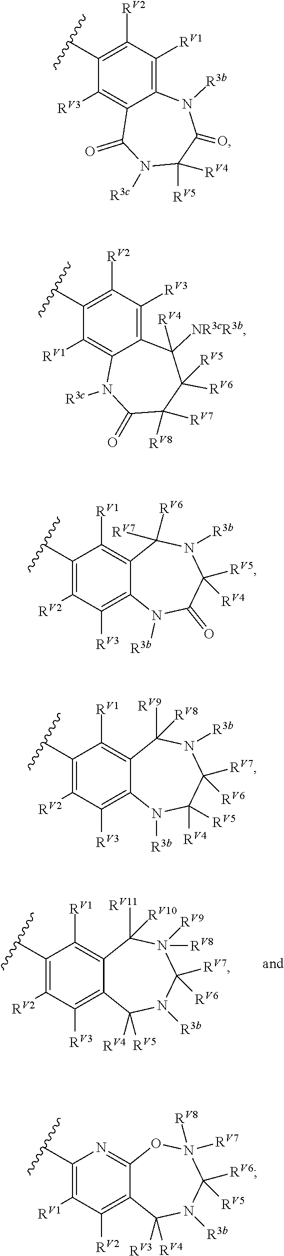

each occurrence of X⁴ is independently selected from the group consisting of CR^{X4} and N, wherein each occurrence of R^{X4} is independently selected from the group consisting of H, C₁-C₃ alkyl, C₃-C₈ cycloalkyl, C₁-C₃ alkoxy, cyano, halogen, C₁-C₃ haloalkyl, and C₁-C₃ haloalkoxy;

R^{2a} is selected from the group consisting of H, halogen, optionally substituted C₁-C₆ alkyl, optionally substituted C₃-C₈ cycloalkyl, —(CH₂)₁₋₃(optionally substituted phenyl), —(CH₂)₁₋₃(optionally substituted heteroaryl), —OR¹, —SR, —NRWRW, —O(CH₂)₁₋₃(optionally substituted phenyl), —O(CH₂)₁₋₃(optionally substituted heteroaryl), —(CH₂)₁₋₃C(═O)OR¹, —(CH₂)₁₋₃C(═O)NR¹R¹, —O(CH₂)₁₋₃C(═O)OR¹, and —O(CH₂)₁₋₃C(═O)NR¹R¹,
wherein each occurrence of R¹ is independently H, C₁-C₆ alkyl, or C₃-C⁸ cycloalkyl, wherein the alkyl or cycloalkyl is independently optionally substituted with at least one of halogen, —OH, C₁-C₆ alkoxy, —NH₂, —NH(C₁-C₆ alkyl), and —N(C₁-C₆ alkyl)(C₁-C₆ alkyl),
or two R¹ can combine with the N atom to which they are bound to form 3-membered to 8-membered optionally substituted heterocyclyl;

R^{3a} is selected from the group consisting of —CHO, —C(O)OR^{III}, —C(═O)NR^{III}R^{III}R, —C(═NR⁵)NR^{III}R^{III}, optionally substituted heterocyclyl, —(CH₂)₁₋₃(optionally substituted heterocyclyl), optionally substituted C₁-C₆ alkoxy, optionally substituted C₁-C₆ aminoalkyl, and optionally substituted C₁-C₆ hydroxyalkyl,
wherein each occurrence of R^{III} is independently H, C₁-C₆ alkyl, or C₃-C⁸ cycloalkyl, wherein the alkyl or cycloalkyl is independently optionally substituted with at least one of halogen, —OH, C₁-C₆ alkoxy, —NH₂, —NH(C₁-C₆ alkyl), and —N(C₁-C₆ alkyl)(C₁-C₆ alkyl),
wherein each occurrence of R⁵ is independently H, C₁-C₆ alkyl, or C₃-C⁸ cycloalkyl, wherein the alkyl or cycloalkyl is independently optionally substituted with at least one of halogen, —OH, C₁-C₆ alkoxy, —NH₂, —NH(C₁-C₆ alkyl), and —N(C₁-C₆ alkyl)(C₁-C₆ alkyl),
or two R^{III} can combine with the N atom to which they are bound to form 3-membered to 8-membered optionally substituted heterocyclyl,
or, if R^{3a} is —C(═NR⁵)NR^{III}R^{III}, then R⁵ and one R^{III} can combine to form 4-membered to 8-membered optionally substituted heterocyclyl,
or, if X³ is CR^{X3} and R^{3a} has a tetravalent a carbon, then optionally R^{X3} and the tetravalent a carbon of R^{3a} combine to form a 3-membered to to 8-membered carbocyclyl;

R^{3b} is selected from the group consisting of H, optionally substituted C₁-C₆ alkyl, optionally substituted C₁-C₆ alkoxy, optionally substituted C₁-C₆ aminoalkyl, optionally substituted C₁-C₆ hydroxyalkyl, optionally substituted C₃-C₈ cycloalkyl, —CHO, —C(O)OR, —C(═O)NR^{IV}R^{IV}, —C(═NR⁵)NR^{IV}R^{IV}, —NR^{3c}(optionally substituted alkyl), optionally substituted heterocyclyl, and —(CH₂)₁₋₃(optionally substituted heterocyclyl),
wherein each occurrence of R^{IV} is independently H, C₁-C₆ alkyl, or C₃-C⁸ cycloalkyl, wherein the alkyl or cycloalkyl is independently optionally substituted with at least one of halogen, —OH, C₁-C₆ alkoxy, —NH₂, —NH(C₁-C₆ alkyl), and —N(C₁-C₆ alkyl)(C₁-C₆ alkyl),
wherein each occurrence of R⁵ is independently H, C₁-C₆ alkyl, or C₃-C⁸ cycloalkyl, wherein the alkyl or cycloalkyl is independently optionally substituted with at least one of halogen, —OH, C₁-C₆ alkoxy, —NH₂, —NH(C₁-C₆ alkyl), and —N(C₁-C₆ alkyl)(C₁-C₆ alkyl),
or two R^{IV} can combine with the N atom to which they are bound to form 3-membered to 8-membered optionally substituted heterocyclyl,
or, if R^{3b} is —C(═NR⁵)NR^{IV}R^{IV}, then R⁵ and one R^{IV} can combine to form 4-membered to 8-membered optionally substituted heterocyclyl;

each occurrence of R³ is independently selected from the group consisting of H, optionally substituted C₁-C₆ alkyl, optionally substituted C₃-C₈ cycloalkyl, and optionally substituted C₁-C₆ acyl;

each occurrence of R^{3d} is independently selected from the group consisting of H, optionally substituted C₁-C₆ alkyl, optionally substituted C₃-C₈ cycloalkyl, and optionally substituted C₁-C₆ acyl;

R^{4a} is selected from the group consisting of H, halogen, cyano, C₁-C₃ alkyl, and C₃-C₈ cycloalkyl;

each occurrence of R^{V1}, R^{V2}, R^{V3}, R^{V4}, R^{V5}, R^{V6}, R^{V7}, R^{V8}, R^{V9}, RV¹⁰, and RV¹¹ is independently selected from the group consisting of H, C₁-C₃ alkyl, C₃-C₈ cycloalkyl, C₁-C₃ alkoxy, cyano, halogen, C₁-C₃ haloalkyl, and C₁-C₃ haloalkoxy. or a salt, solvate, geometric isomer, stereoisomer, tautomer and any mixtures thereof.

In certain embodiments, the compound of formula (Ib) is:

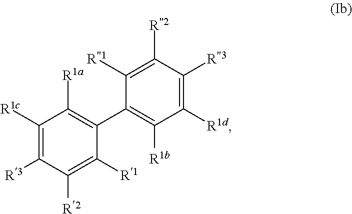

wherein:

R^{1a} is selected from the group consisting of H, C₁-C₃ alkyl, C₃-C₈ cycloalkyl, C₁-C₃ alkoxy, cyano, halogen, C₁-C₃ haloalkyl, and C₁-C₃ haloalkoxy;

R^{1b} is selected from the group consisting of H, C₁-C₃ alkyl, C₃-C₈ cycloalkyl, C₁-C₃ alkoxy, cyano, halogen, C₁-C₃ haloalkyl, and C₁-C₃ haloalkoxy;

R^{1c} and R^{1d} are independently selected from the group consisting of:

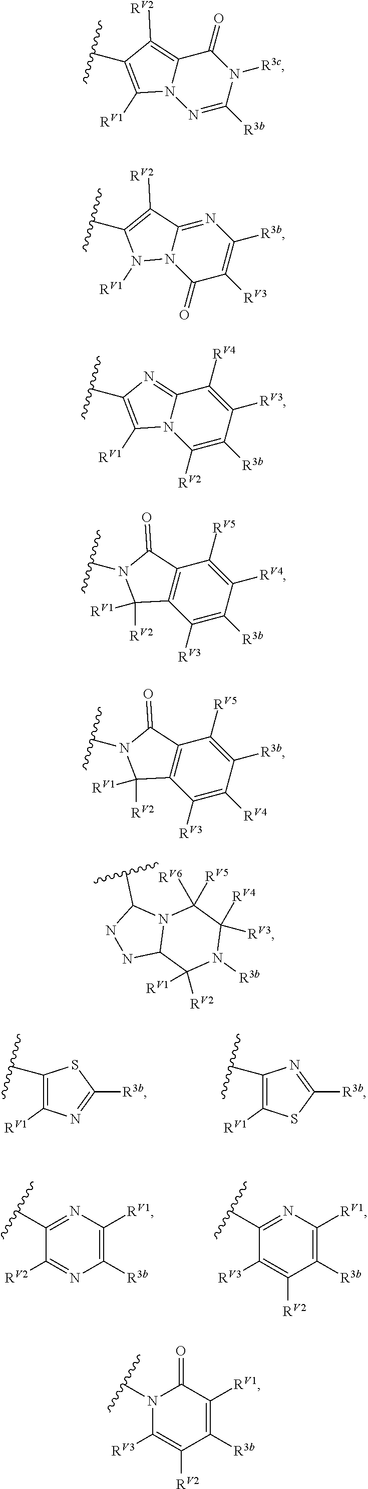

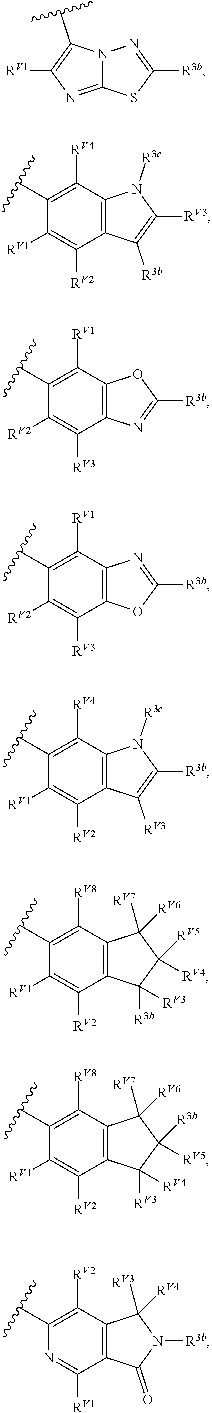

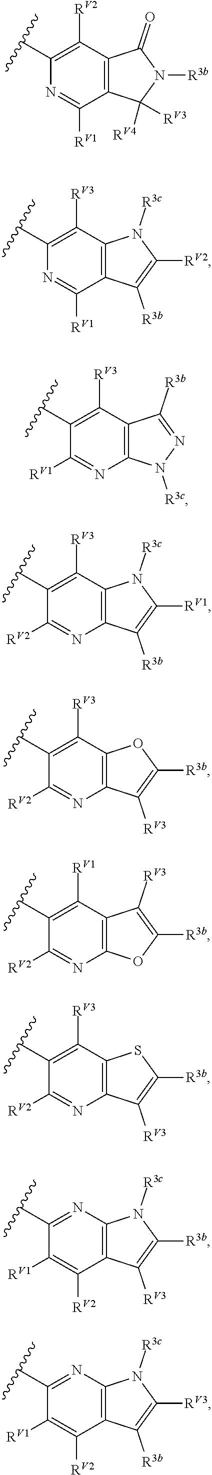

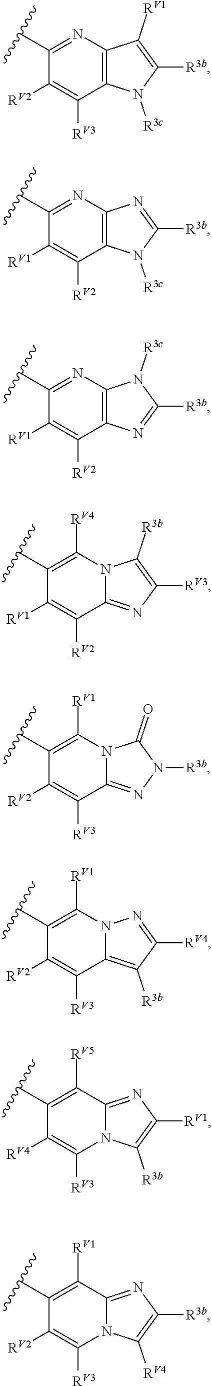

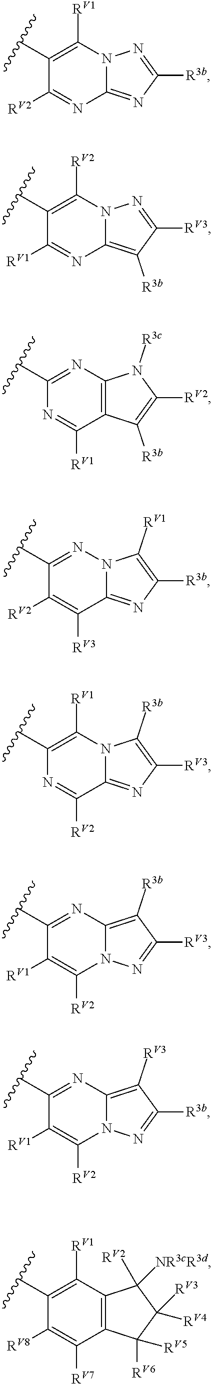

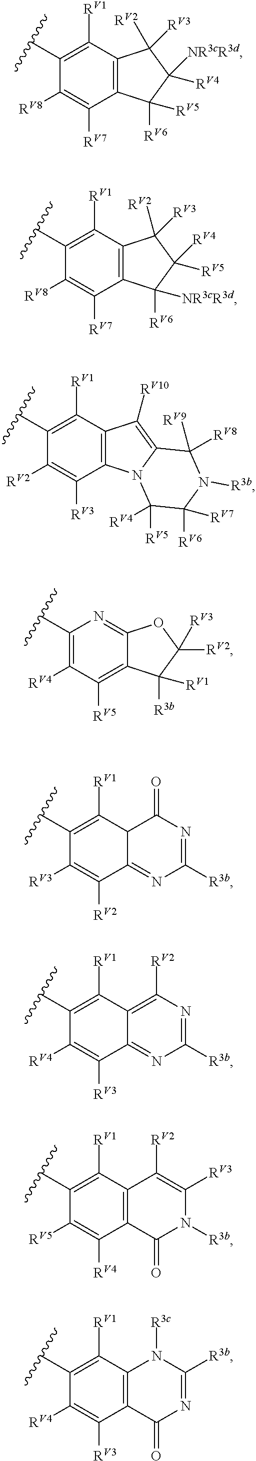

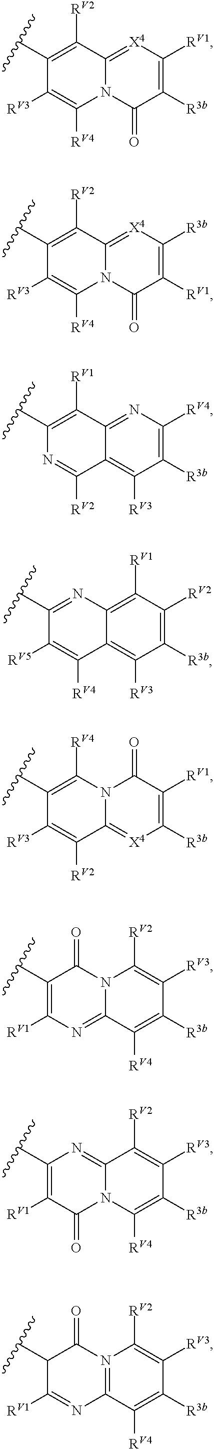

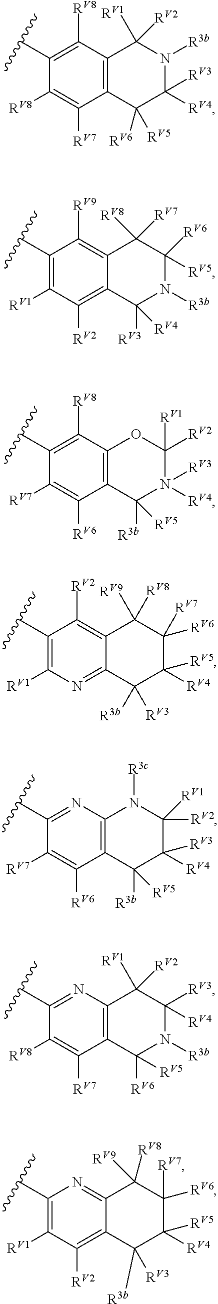

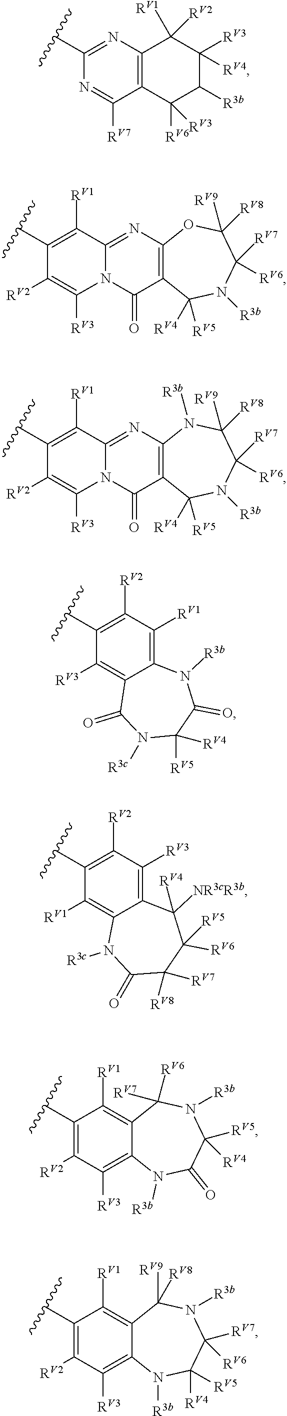

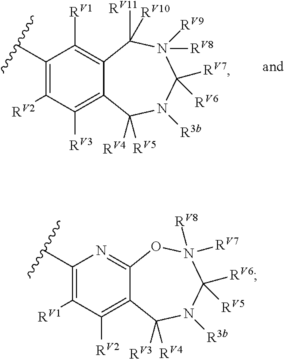

R′¹, R′², and R′³ are independently selected from the group consisting of H, C₁-C₃ alkyl, C₃-C₈ cycloalkyl, C₁-C₃ alkoxy, cyano, halogen, C₁-C₃ haloalkyl, and C₁-C₃ haloalkoxy;

R″¹, R″², and R″³ are independently selected from the group consisting of H, C₁-C₃ alkyl, C₃-C₈ cycloalkyl, C₁-C₃ alkoxy, cyano, halogen, C₁-C₃ haloalkyl, and C₁-C₃ haloalkoxy;

each occurrence of X⁴ is independently selected from the group consisting of CR^{X4} and N, wherein each occurrence of R^{X4} is selected from the group consisting of H, C₁-C₃ alkyl, C₃-C₈ cycloalkyl, C₁-C₃ alkoxy, cyano, halogen, C₁-C₃ haloalkyl, and C₁-C₃ haloalkoxy;

each occurrence of R^{3b} is independently selected from the group consisting of H, optionally substituted C₁-C₆ alkyl, optionally substituted C₁-C₆ alkoxy, optionally substituted C₁-C₆ aminoalkyl, optionally substituted C₁-C₆ hydroxyalkyl, optionally substituted C₃-C₈ cycloalkyl, —CHO, —C(O)OR^{IV}, —C(═O)NR^{IV}R^{IV}, —C(═NR⁵)NR^{IV}R^{IV}, —NR^{3d}(optionally substituted alkyl), optionally substituted heterocyclyl, and —(CH₂)₁₋₃(optionally substituted heterocyclyl),
wherein each occurrence of R^{IV} is independently H, C₁-C₆ alkyl, or C₃-C⁸ cycloalkyl, wherein the alkyl or cycloalkyl is independently optionally substituted with at least one of halogen, —OH, C₁-C₆ alkoxy, —NH₂, —NH(C₁-C₆ alkyl), and —N(C₁-C₆ alkyl)(C₁-C₆ alkyl),
wherein each occurrence of R⁵ is independently H, C₁-C₆ alkyl, or C₃-C⁸ cycloalkyl, wherein the alkyl or cycloalkyl is independently optionally substituted with at least one of halogen, —OH, C₁-C₆ alkoxy, —NH₂, —NH(C₁-C₆ alkyl), and —N(C₁-C₆ alkyl)(C₁-C₆ alkyl),
or two R^{IV} can combine with the N atom to which they are bound to form 3-membered to 8-membered optionally substituted heterocyclyl,
or, if R^{3b} is —C(═NR⁵)NR^{IV}R^{IV}, then R⁵ and one R^{IV} can combine to form 4-membered to 8-membered optionally substituted heterocyclyl;

each occurrence of R^{3c} is independently selected from the group consisting of H, optionally substituted C₁-C₆ alkyl, optionally substituted C₃-C₈ cycloalkyl, and optionally substituted C₁-C₆ acyl;

each occurrence of R^{3d} is independently selected from the group consisting of H, optionally substituted C₁-C₆ alkyl, optionally substituted C₃-C₈ cycloalkyl, and optionally substituted C₁-C₆ acyl;

each occurrence of R^{V1}, R^{V2}, R^{V3}, R^{V4}, R^{V5}, R^{V6}, R^{V7}, R^{V8}, R^{V9}, R^{V10}, and R^{V11} is independently selected from the group consisting of H, C₁-C₃ alkyl, C₃-C₈ cycloalkyl, C₁-C₃ alkoxy, cyano, halogen, C₁-C₃ haloalkyl, and C₁-C₃ haloalkoxy. or a salt, solvate, geometric isomer, stereoisomer, tautomer and any mixtures thereof.

In certain embodiments, the compound of formula (Ia) is:

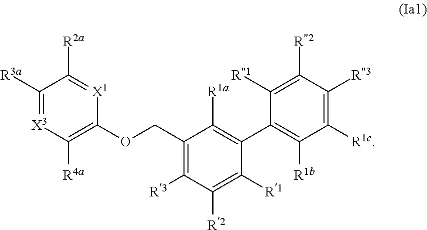

In certain embodiments, the compound of formula (Ia) is:

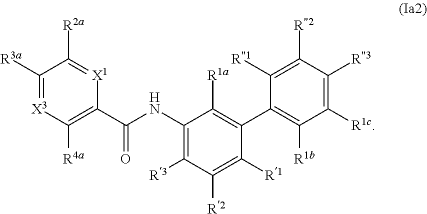

In certain embodiments, the compound of formula (Ia) is:

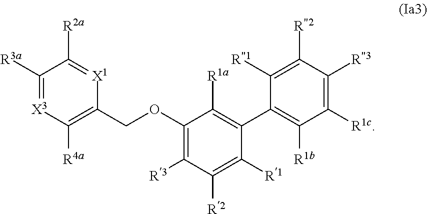

In certain embodiments, the compound of formula (Ia) is:

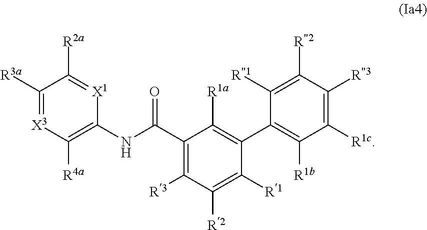

In certain embodiments, the compound of formula (Ia) is:

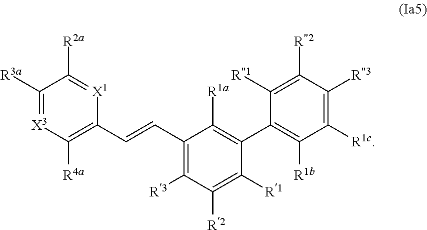

In certain embodiments, the compound of formula (Ia) is:

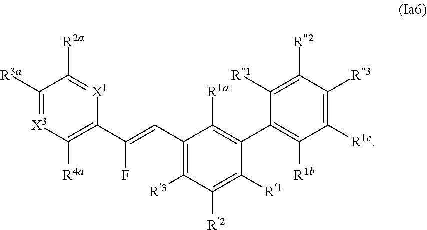

In certain embodiments, the compound of formula (Ia) is:

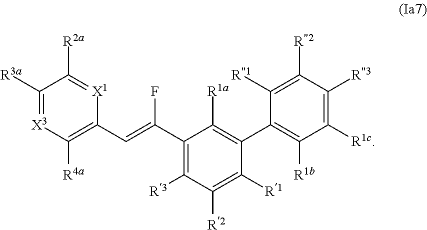

In certain embodiments, the compound of formula (Ia) is:

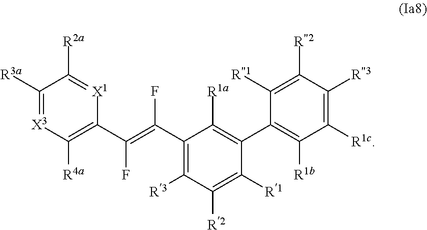

In certain embodiments, X¹ is CR^{X1}. In certain embodiments, X¹ is N.

In certain embodiments, X² is bond. In certain embodiments, X² is —OCH₂—**. In certain embodiments, X² is —CH₂O—**. In certain embodiments, X² is —CH═CH—**. In certain embodiments, X² is —CF═CH—**. In certain embodiments, X² is —CH═CF—**. In certain embodiments, X² is —CF═CF—**. In certain embodiments, X² is —C(═O)NH—**. In certain embodiments, X² is —NHC(═O)—**.

In certain embodiments, X³ is CR^{X3}. In certain embodiments, X³ is N.

In certain embodiments, X⁴ is CR^{X4}. In certain embodiments, X⁴ is N.

In certain embodiments, R^{1a} is identical to R^{1b}. In other embodiments, R^{1a} is not identical to R^{1b}. In yet other embodiments, R^{1a} is methyl. In yet other embodiments, R^{1b} is methyl. In yet other embodiments, R^{1a} is H. In yet other embodiments, R^{1b} is H. In yet other embodiments, R^{1a} is Cl. In yet other embodiments, R^{1b} is Cl.

In certain embodiments, R^{1c} is

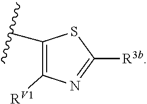

In certain embodiments, R^{1c} is

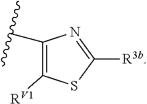

In certain embodiments, R^{1c} is

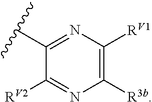

In certain embodiments, R^{1c} is

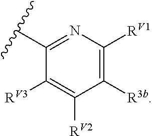

In certain embodiments, R^{1c} is

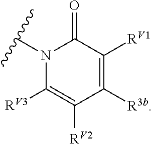

In certain embodiments, R^{1c} is

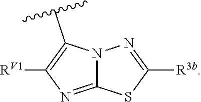

In certain embodiments, R^{1c} is

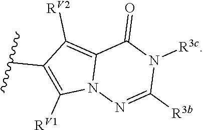

In certain embodiments, R^{1c} is

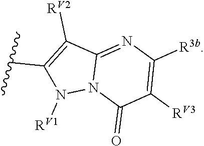

In certain embodiments, R^{1c} is

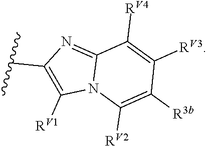

In certain embodiments, R^{1c} is

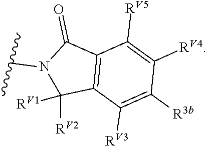

In certain embodiments, R^{1c} is

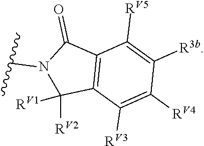

In certain embodiments, R^{1c} is

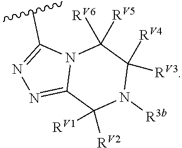

In certain embodiments, R^{1c} is

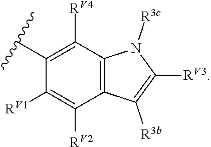

In certain embodiments, R^{1c} is

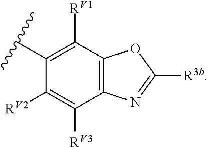

In certain embodiments, R^{1c} is

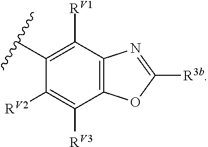

In certain embodiments, R^{1c} is

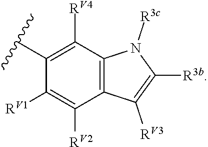

In certain embodiments, R^{1c} is

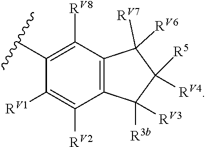

In certain

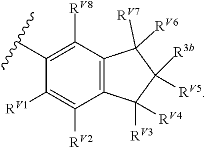

In certain embodiments, R^{1c} is

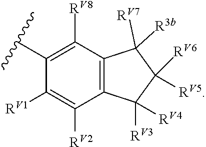

In certain embodiments, R^{1c} is

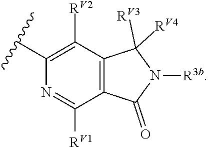

In certain embodiments, R^{1c} is

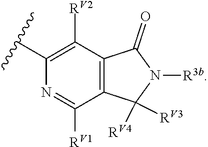

In certain embodiments, R^{1c} is

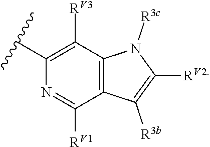

In certain

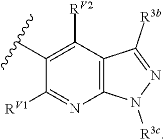

In certain embodiments, R^{1c} is

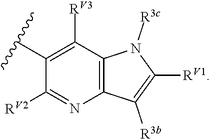

In certain embodiments, R^{1c} is

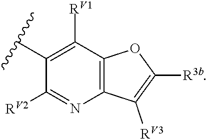

In certain embodiments, R^{1c} is

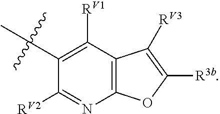

In certain embodiments, R^{1c} is

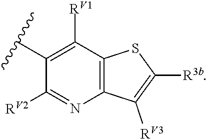

In certain embodiments, R^{1c} is

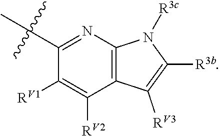

In certain embodiments, R^{1c} is

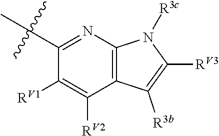

In certain embodiments, R^{1c} is

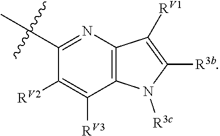

In certain embodiments, R^{1c} is

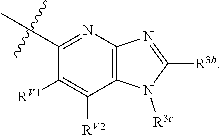

In certain embodiments, R^{1c} is

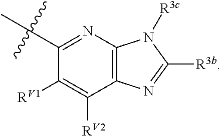

In certain embodiments, R^{1c} is

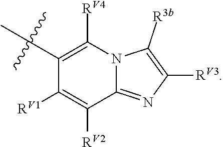

In certain embodiments, R^{1c} is

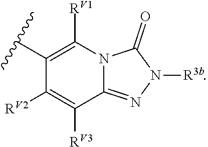

In certain embodiments, R^{1c} is

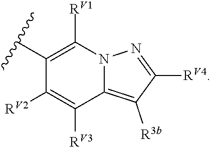

In certain embodiments, R^{1c} is

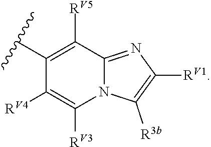

In certain embodiments, R^{1c} is

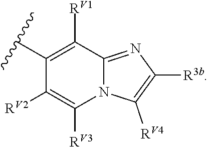

In certain embodiments, R^{1c} is

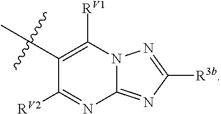

In certain embodiments, R^{1c} is

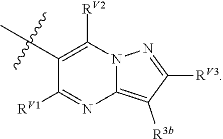

In certain embodiments, R^{1c} is

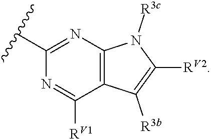

In certain embodiments, R^{1c} is

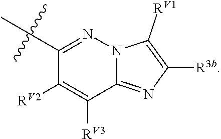

In certain embodiments, R^{1c} is

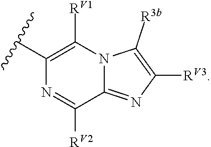

In certain embodiments, R^{1c} is

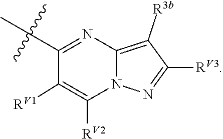

In certain embodiments, R^{1c} is

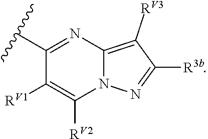

In certain embodiments, R^{1c} is

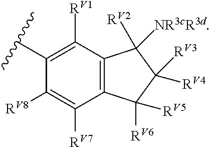

In certain embodiments, R^{1c} is

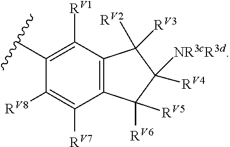

In certain embodiments, R^{1c} is

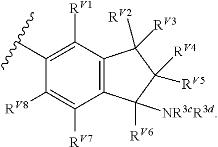

In certain embodiments, R^{1c} is

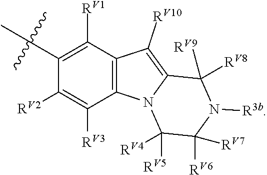

In certain embodiments, R^{1c} is

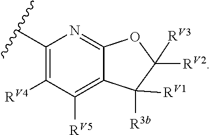

In certain embodiments, R^{1c} is

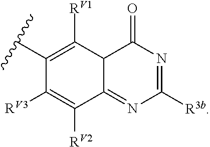

In certain embodiments, R^{1c} is

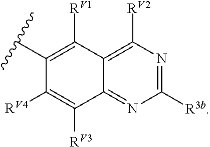

In certain embodiments, R^{1c} is

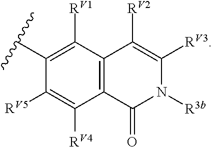

In certain embodiments, R^{1c} is

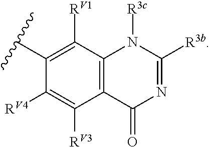

In certain embodiments, R^{1c} is

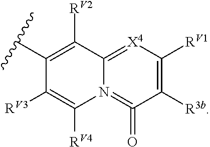

In certain embodiments, R^{1c} is

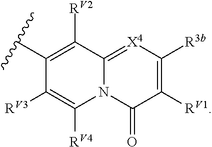

In certain embodiments, R^{1c} is

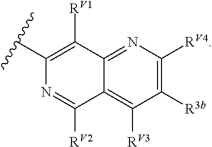

In certain embodiments, R^{1c} is

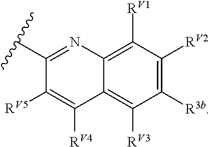

In certain embodiments, R^{1c} is

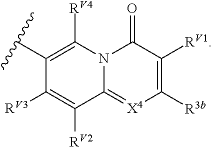

In certain embodiments, R^{1c} is

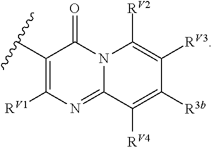

In certain embodiments, R^{1c} is

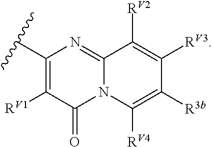

In certain embodiments, R^{1c} is

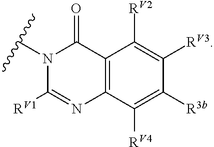

In certain embodiments, R^{1c} is

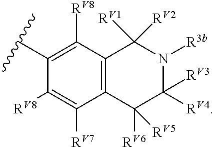

In certain embodiments, R^{1c} is

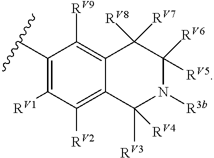

In certain embodiments, R^{1c} is

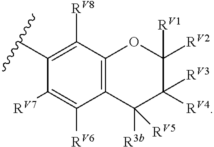

In certain embodiments, R^{1c} is

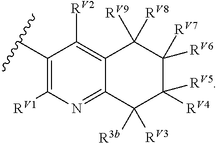

In certain embodiments, R^{1c} is

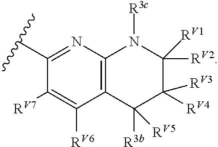

In certain embodiments, R^{1c} is

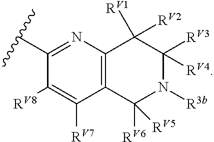

In certain embodiments, R^{1c} is

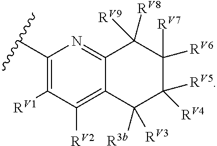

In certain embodiments, R^{1c} is

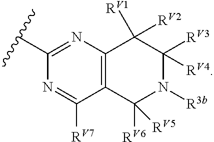

In certain

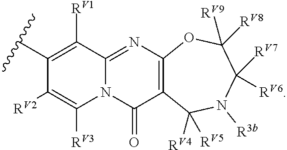

embodiments, R^{1c} is

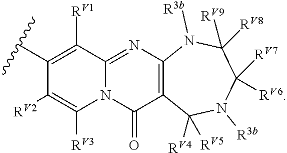

In certain embodiments, R^{1c} is

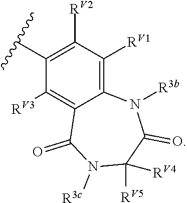

In certain embodiments, R^{1c} is

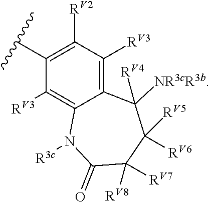

In certain embodiments, R^{1c} is

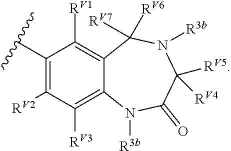

In certain embodiments, R^{1c} is

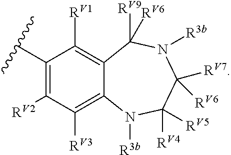

In certain embodiments, R^{1c} is

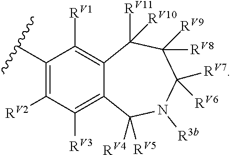

In certain embodiments, R^{1c} is

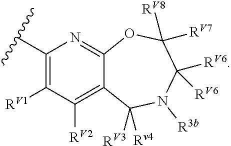

In certain embodiments, R^{1d} is

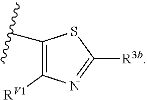

In certain embodiments, R^{1d} is

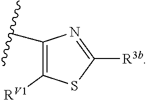

In certain embodiments, R^{1d} is

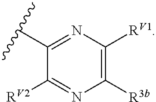

In certain embodiments, R^{1d} is

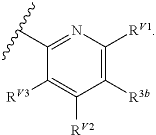

In certain embodiments, R^{1d} is

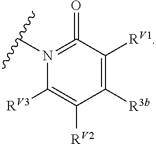

In certain embodiments, R^{1d} is

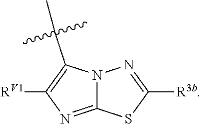

In certain embodiments, R^{1d} is

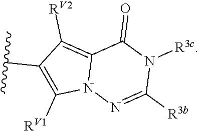

In certain embodiments, R^{1d} is

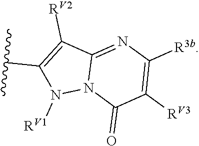

In certain embodiments, R^{1d} is

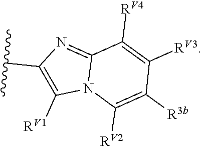

In certain embodiments, R^{1d} is

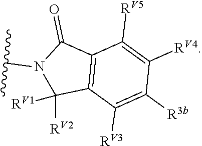

In certain embodiments, R^{1d} is

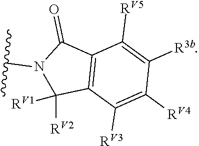

In certain embodiments, R^{1d} is

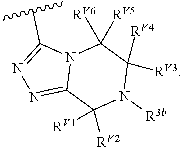

In certain embodiments, R^{1d} is

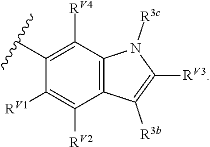

In certain embodiments, R^{1d} is

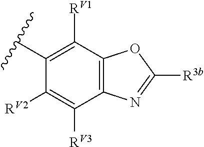

In certain embodiments, R^{1d} is

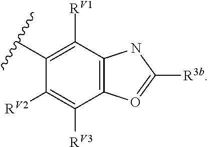

In certain embodiments, R^{1d} is

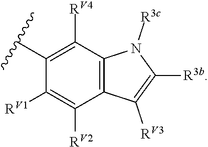

In certain embodiments, R^{1d} is

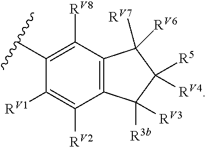

In certain embodiments, R^{1d} is

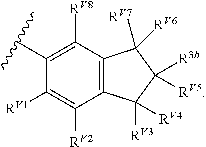

In certain embodiments, R^{1d} is

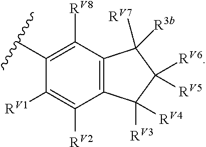

In certain embodiments, R^{1d} is

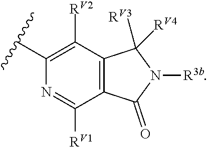

In certain embodiments, R^{1d} is

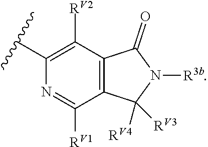

In certain embodiments, R^{1d} is

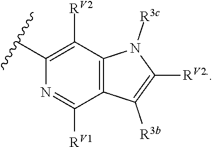

In certain embodiments, R^{1d} is

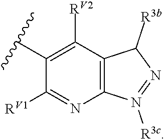

In certain embodiments, R^{1d} is

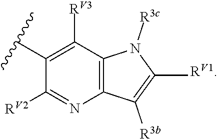

certain embodiments, R^{1d} is

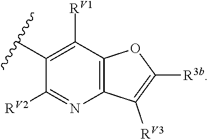

In certain embodiments, R^{1d} is

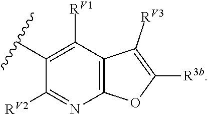

In certain embodiments, R^{1d} is

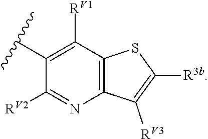

In certain embodiments, R^{1d} is

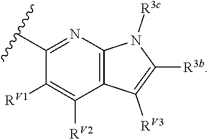

In certain embodiments, R^{1d} is

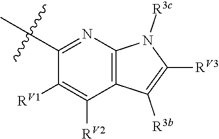

In certain embodiments, R^{1d} is

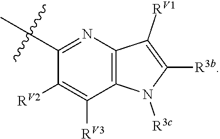

In certain embodiments, R^{1d} is

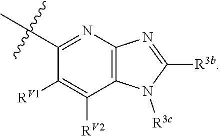

In certain embodiments, R^{1d} is

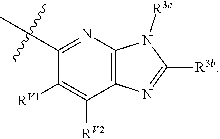

In certain embodiments, R^{1d} is

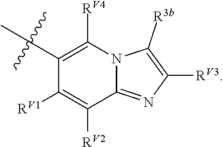

In certain embodiments, R^{1d} is

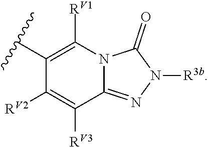

In certain embodiments, R^{1d} is

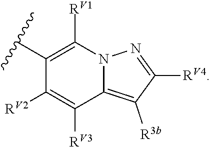

In certain embodiments, R^{1d} is

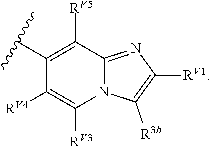

In certain embodiments, R^{1d} is

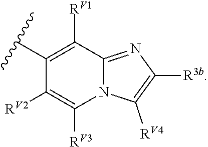

In certain embodiments, R^{1d} is

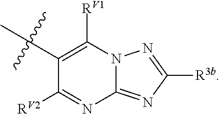

In certain embodiments, R^{1d} is

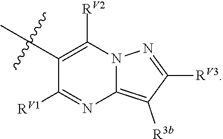

In certain embodiments, R^{1d} is

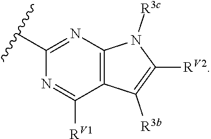

In certain embodiments, R^{1d} is

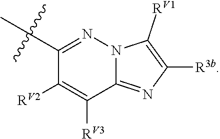

In certain embodiments, R^{1d} is

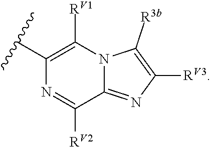

In certain embodiments, R^{1d} is

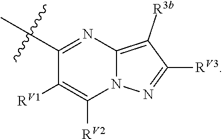

In certain embodiments, R^{id} is

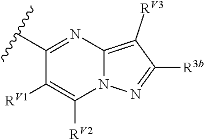

In certain embodiments, R^{1d} is

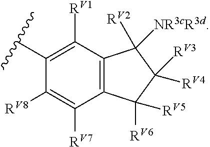

In certain embodiments, R^{1d} is

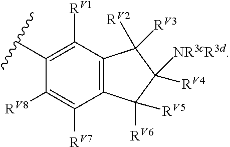

In certain embodiments, R^{1d} is

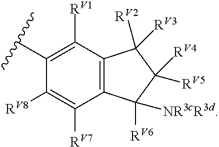

In certain embodiments, R^{1d} is

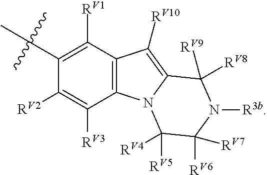

In certain embodiments, R^{1d} is

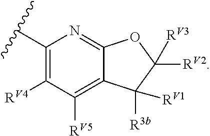

In certain embodiments, R^{1d} is

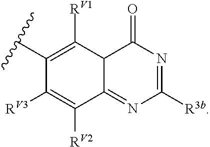

In certain

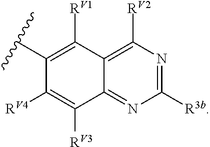

embodiments, R^{1d} is
In certain embodiments, R^{1d} is

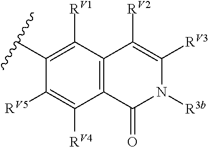

In certain embodiments, R^{1d} is

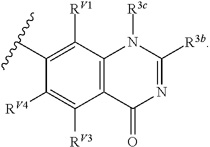

In certain embodiments, R^{1d} is

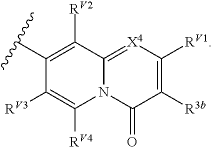

In certain embodiments, R^{1d} is

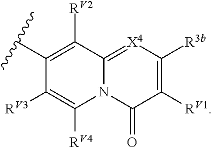

In certain embodiments, R^{1d} is

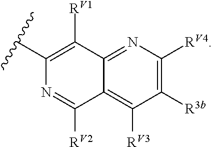

In certain embodiments, R^{1d} is

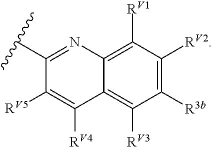

In certain embodiments, R^{1d} is

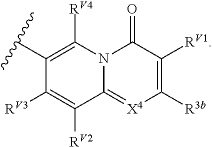

In certain embodiments, R^{1d} is

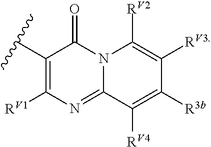

In certain embodiments, R^{1d} is

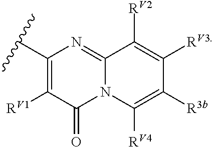

In certain embodiments, R^{1d} is

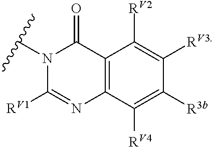

In certain embodiments, R^{1d} is

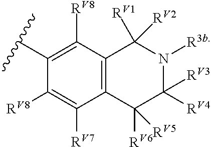

In certain embodiments, R^{1d} is

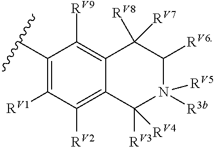

In certain embodiments, R^{1d} is

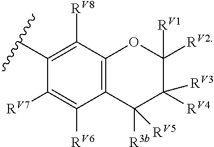

In certain embodiments, R^{1d} is

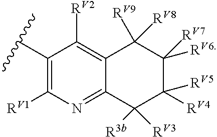

In certain embodiments, R^{1d} is

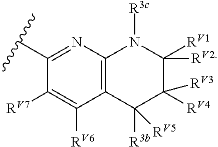

In certain embodiments, R^{1d} is

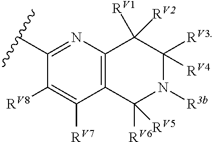

In certain embodiments, R^{1d} is

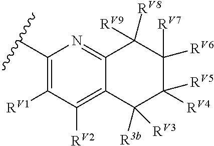

In certain embodiments, R^{1d} is

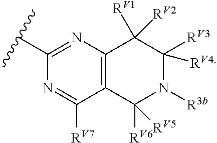

In certain embodiments, R^{1d} is

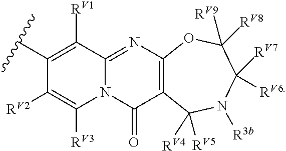

In certain embodiments, R^{1d} is

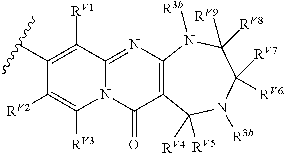

In certain embodiments, R^{1d} is

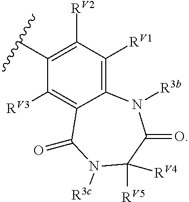

In certain embodiments, R^{1d} is

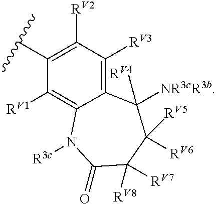

In certain embodiments, R^{1d} is

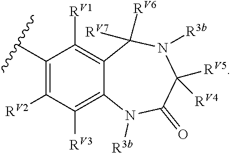

In certain embodiments, R^{1d} is

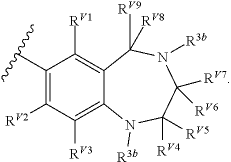

In certain embodiments, R^{1d} is

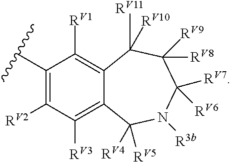

In certain embodiments, R^{1d} is

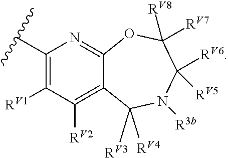

In certain embodiments, R^{1c} is

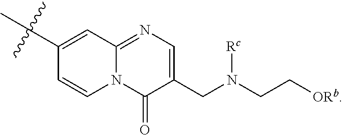

In certain embodiments,

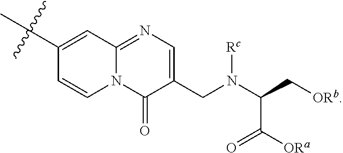

In certain embodiments, R^{1c} is

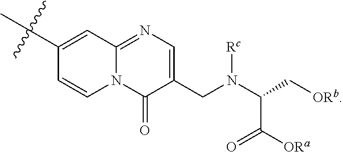

In certain embodiments, R^{1c} is

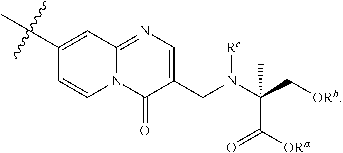

In certain embodiments, R^{1c} is

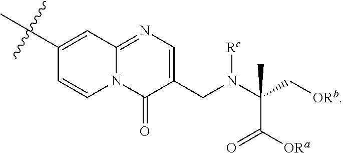

In certain embodiments, R^{1c} is

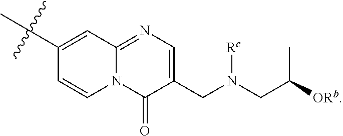

In certain embodiments, R^{1c} is

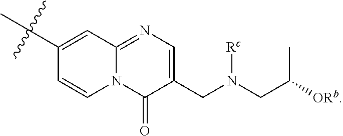

In certain embodiments, R^{1c} is

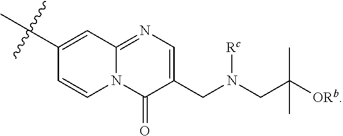

In certain embodiments, R^{1c} is

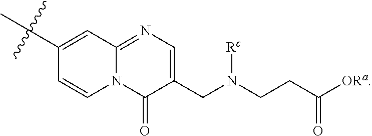

In certain embodiments, R^{1c} is

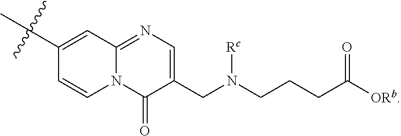

In certain embodiments, R^{1c} is

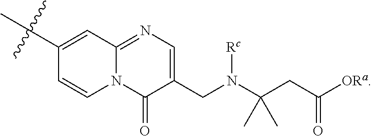

In certain embodiments, R^{1c} is

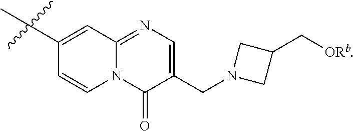

In certain embodiments, R^{1c} is

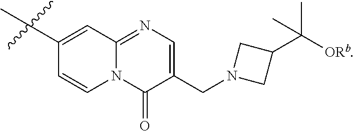

In certain embodiments, R^{1c} is

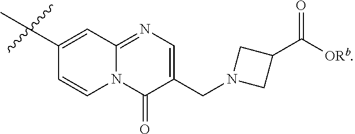

In certain embodiments, R^{1c} is

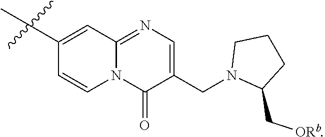

In certain embodiments, R^{1c} is

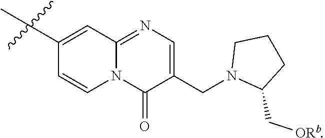

In certain embodiments, R^{1c} is

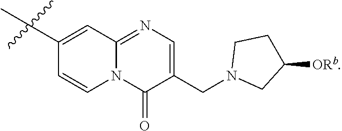

In certain embodiments, R^{1c} is

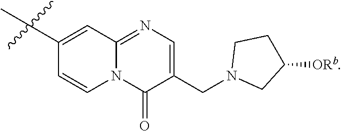

In certain embodiments, R^{1c} is

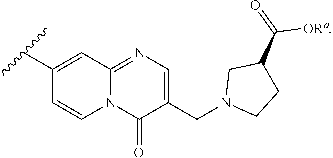

In certain embodiments, R^{1c} is

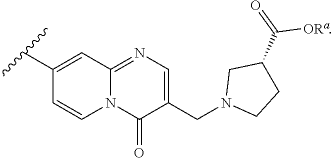

In certain embodiments, R^{1c} is

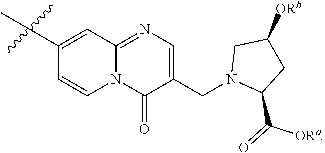

certain embodiments, R^{1c} is

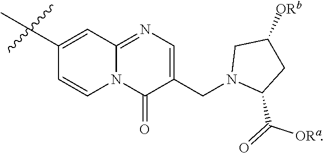

In certain embodiments, R^{1c} is

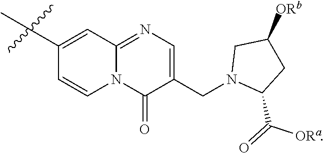

In certain embodiments, R^{1c} is

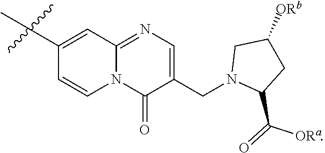

In certain embodiments, R^{1c} is

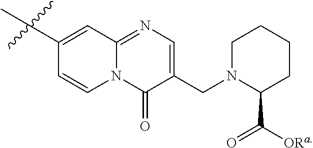

In certain embodiments, R^{1c} is

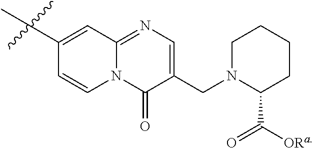

In certain embodiments, R^{1c} is

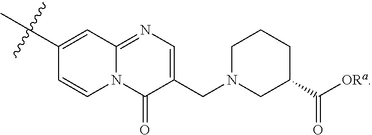

In certain embodiments, R^{1c} is

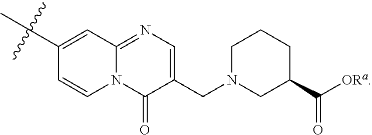

In certain embodiments, R^{1c} is

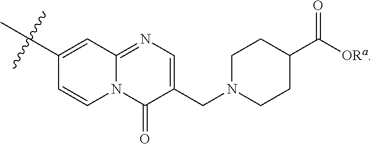

In certain embodiments, R^{1c} is

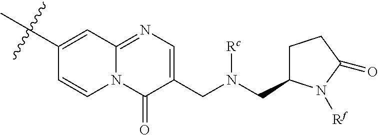

In certain embodiments R^{1c} is

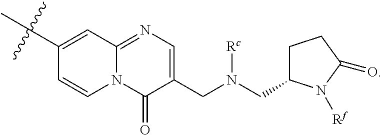

In certain embodiments, R^{1c} is

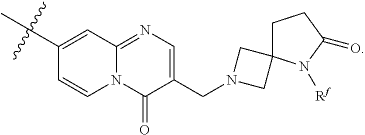

In certain embodiments, R^{1c} is

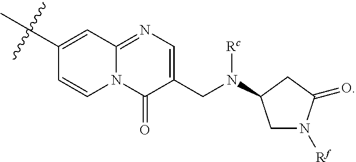

In certain embodiments, R^{1c} is

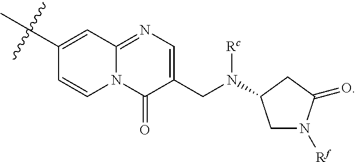

In certain embodiments, R^{1c} is

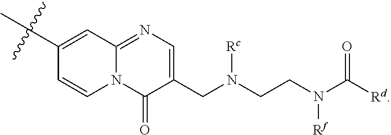

In certain embodiments, R^{1c} is

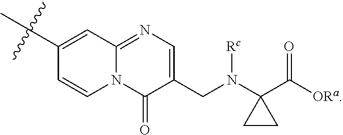

In certain embodiments, R^{1c} is

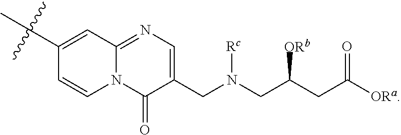

In certain embodiments, R^{1c} is

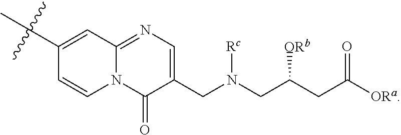

In certain embodiments, R^{1c} is

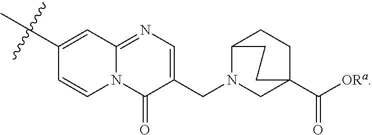

In certain embodiments, R^{1c} is

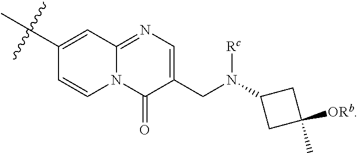

In certain embodiments, R^{1c} is

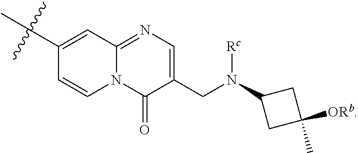

In certain embodiments, R^{1c} is

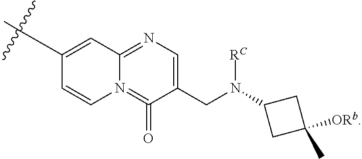

In certain embodiments, R^{1c} is

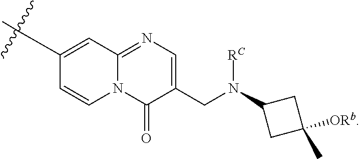

In certain embodiments, R^{1c} is

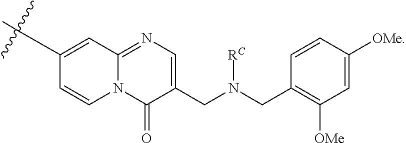

In certain embodiments, R^{1c} is

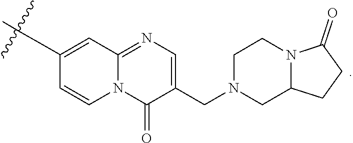 [SMILES: *c1ccn2c(=O)c(CN3CCN4C(=O)CCC4C3)cnc2c1]

In certain embodiments, R^{1c} is

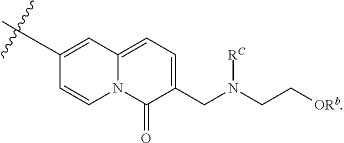

In certain embodiments R^{1c} is

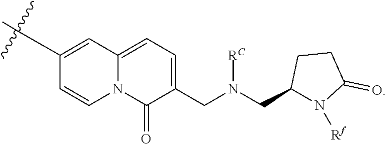

In certain embodiments, R^{1c} is

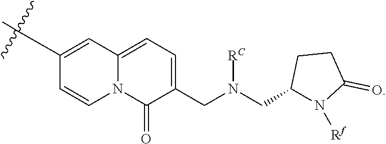

In certain embodiments, R^{1c} is

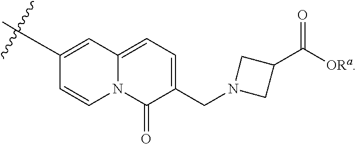

In certain embodiments, R^{1c} is

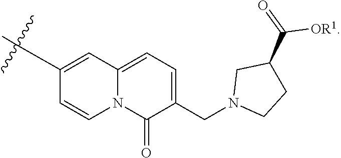

In certain embodiments, R^{1c} is

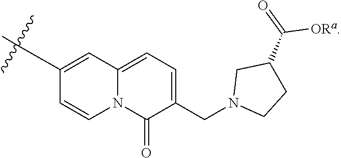

In certain embodiments, R^{1c} is

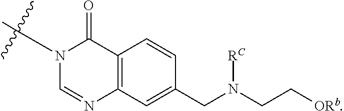

In certain embodiments, R^{1c} is

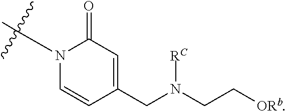

In certain embodiments, R^{1c} is

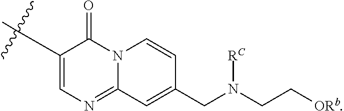

In certain embodiments, R^{1c} is

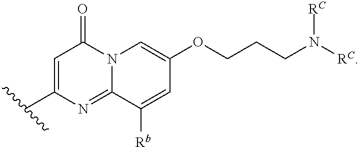

In certain embodiments, R^{1c} is

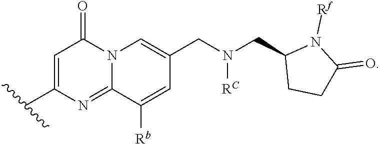

In certain embodiments, R^{1c} is

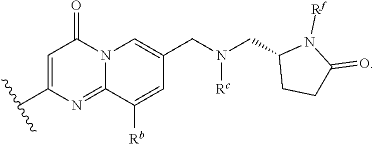

In certain embodiments, R^{1c} is

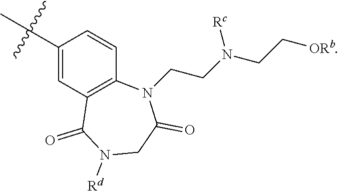

In certain embodiments, R^{1c} is

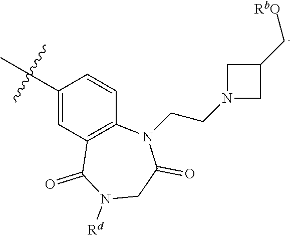

In certain embodiments, R^{1c} is

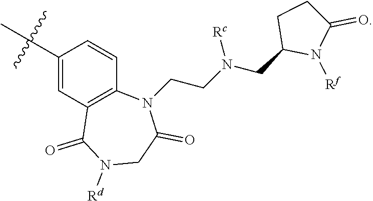

In certain embodiments, R^{1c} is

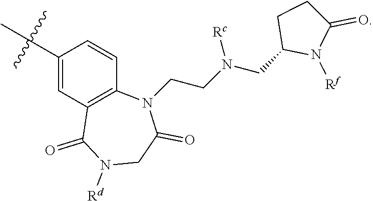

In certain embodiments, R^{1c} is

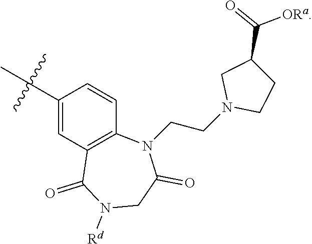

In certain embodiments, R^{1c} is

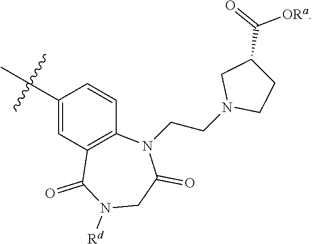

In certain embodiments, R^{1c} is

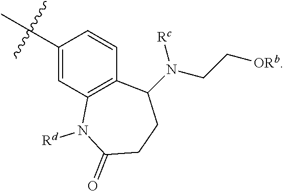

In certain embodiments, R^{1c} is

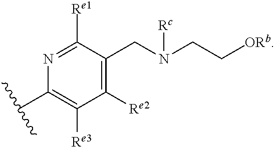

In certain embodiments, R^{1c} is

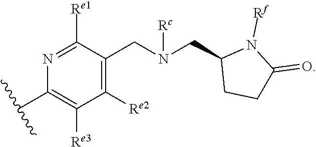

In certain embodiments, R^{1c} is

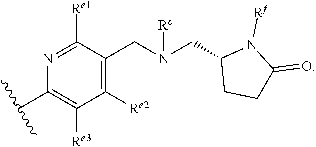

In certain embodiments, R^{1c} is

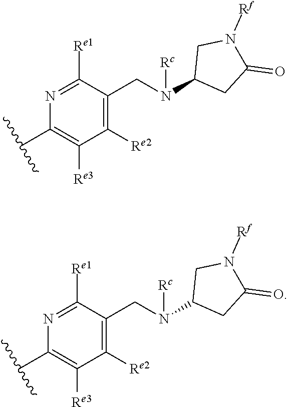

In certain embodiments, R^{1c} is

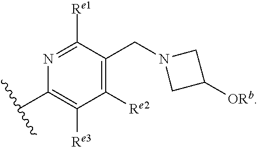

In certain embodiments, R^{1c} is

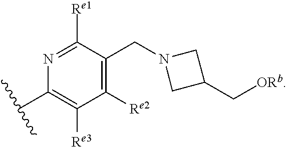

In certain embodiments, R^{1c} is

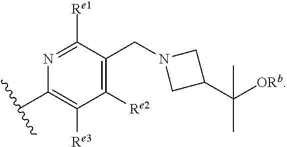

In certain embodiments, R^{1c} is

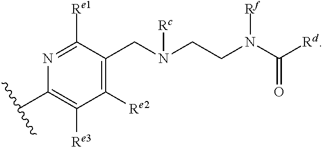

In certain embodiments, R^{1c} is

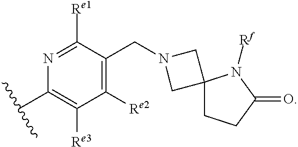

In certain embodiments, R^{1c} is

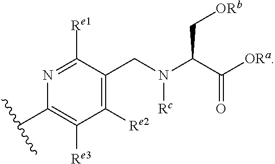

In certain embodiments, R^{1c} is

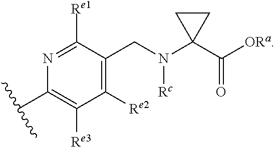

In certain embodiments, R^{1c} is

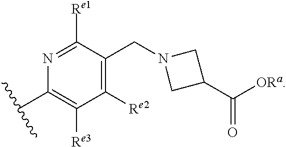

In certain embodiments R^{1c} is

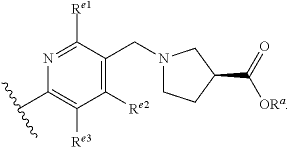

In certain embodiments R^{1c} is

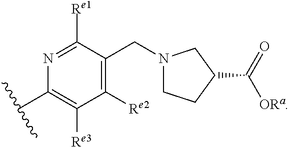

In certain embodiments, R^{1c} is

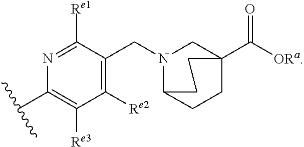

In certain embodiments, R^{1c} is

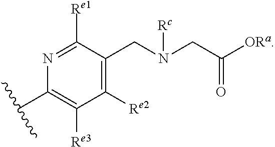

In certain embodiments, R^{1c} is

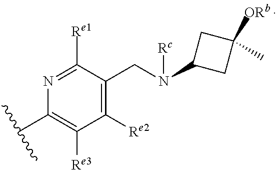

In certain embodiments, R^{1c} is

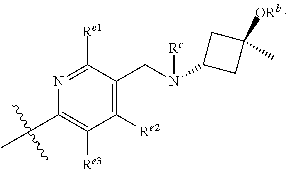

In certain embodiments, R^{1c} is

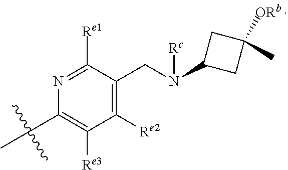

In certain embodiments, R^{1c} is

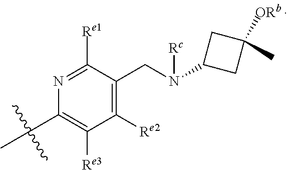

In certain embodiments, R^{1c} is

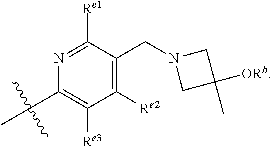

In certain embodiments, R^{1c} is

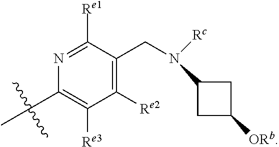

In certain embodiments, R^{1c} is

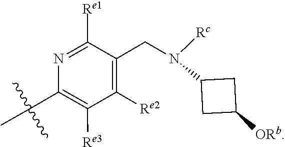

In certain embodiments, R^{1c} is

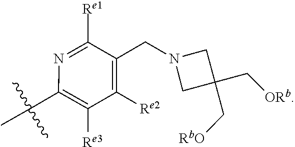

In certain embodiments, R^{1c} is

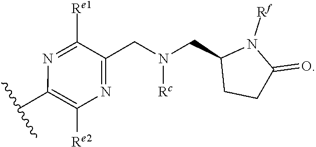

In certain embodiments, R^{1c} is

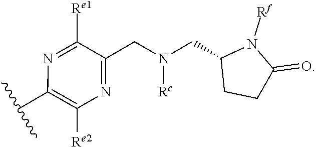

In certain embodiments, R^{1c} is

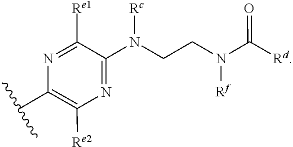

In certain embodiments, R^{1c} is

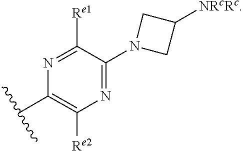

In certain embodiments, R^{1c} is

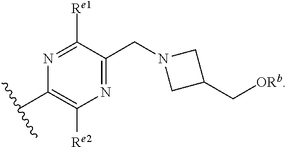

In certain embodiments R^{1c} is

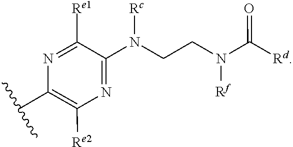

In certain embodiments, R^{1c} is

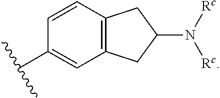

In certain embodiments, R^{1C} is

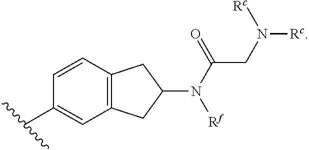

In certain embodiments, R^{1c} is

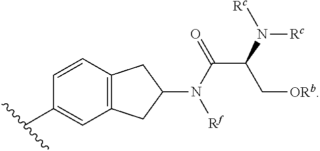

In certain embodiments, R^{1c} is

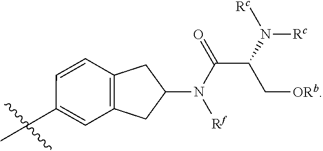

In certain embodiments, R^{1c} is

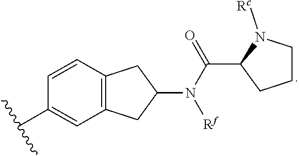

In certain embodiments, R^{1c} is

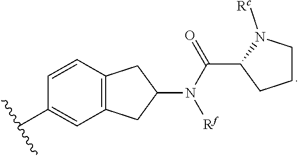

In certain embodiments, R^{1c} is

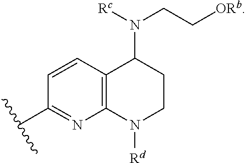

In certain embodiments, R^{1c} is

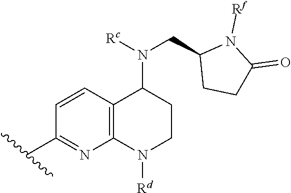

In certain embodiments, R^{1c} is

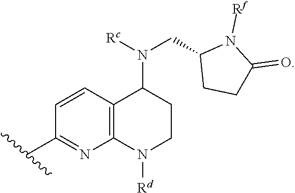

In certain embodiments, R^{1c} is

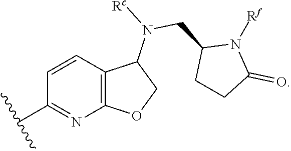

In certain embodiments, R^{1c} is

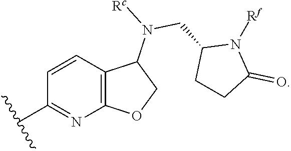

In certain embodiments, R^{1c} is

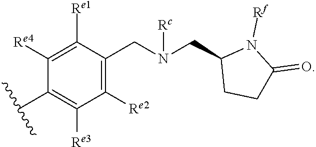

In certain embodiments, R^{1c} is

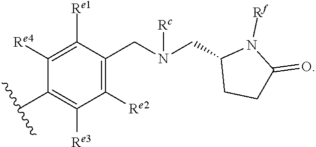

In certain embodiments, R^{1c} is

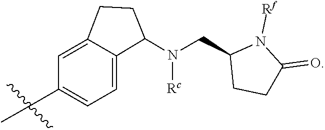

In certain embodiments, R^{1c} is

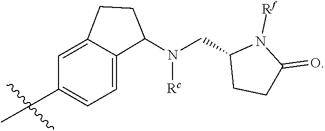

In certain embodiments, R^{1c} is

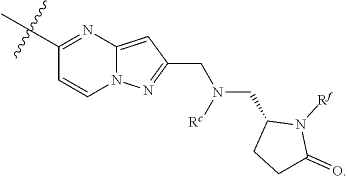

In certain embodiments, R^{1c} is

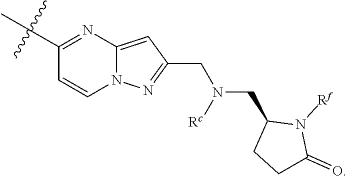

In certain embodiments, R^{1c} is

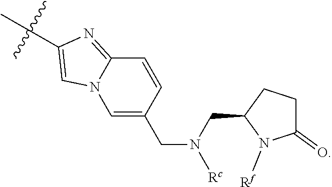

In certain embodiments, R^{1c} is

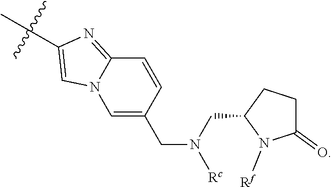

In certain embodiments, R^{1c} is

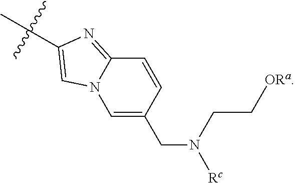

In certain embodiments, R^{1c} is

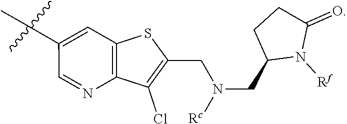

In certain embodiments, R^{1c} is

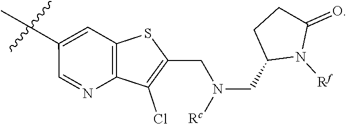

In certain embodiments, R^{1c} is

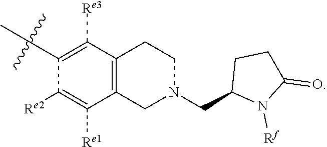

In certain embodiments, R^{1c} is

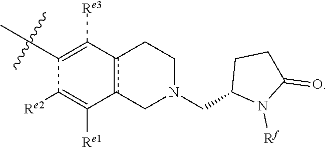

In certain embodiments, each occurrence of R^{a}, R^{b}, R^{c}, R^{d}, and R^{f} is independently selected from the group consisting of H, optionally substituted C₁-C₆ alkyl, and optionally substituted C₃-C₈ cycloalkyl. In certain embodiments, each occurrence of R^{e1}, R^{e2}, R^{e3}, and R^{e4} is independently selected from the group consisting of H, F, C₁, Br, C₁-C₆ alkyl, C₁-C₆ haloalkyl (such as but not limited to —CHF₂ and —CF₃), C₁-C₆ alkoxy, C₁-C₆ haloalkoxy (such as but not limited to —OCHF₂ and —OCF₃), C₁-C₆ thioalkoxy, C₃-C₈ cycloalkyl, C₃-C₈ halocycloalkyl, C₃-C₈ cycloalkoxy, C₃-C₈ halocycloalkoxy, C₃-C₈ thioalkoxy, and NR^{g}R^{g}, wherein each occurrence of R^{g} is independently H, optionally substituted C₁-C₆ alkyl, or optionally substituted C₃-C₈ cycloalkyl. In certain embodiments, at least one of R^{e1} and R^{e2} is not H. In certain embodiments, R^{e1} is not H. In certain embodiments, R^{e2} is not H.

In certain embodiments, R^{1d} is

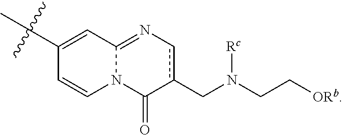

In certain embodiments, R^{1d} is

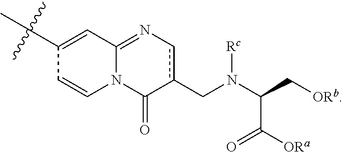

In certain embodiments, R^{1d} is

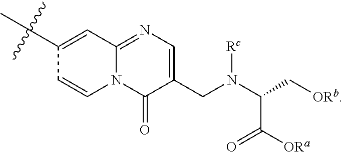

In certain embodiments, R^{1d} is

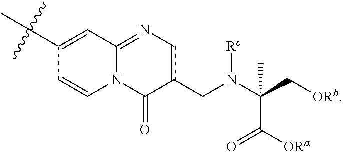

In certain embodiments R^{1d} is

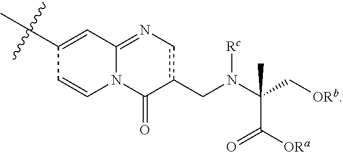

In certain embodiments, R^{1d} is

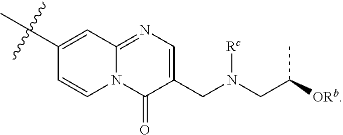

In certain embodiments, R^{1d} is

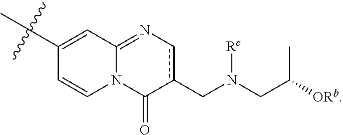

In certain embodiments, R^{1d} is

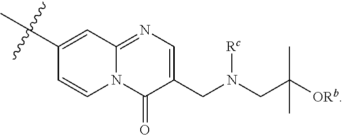

In certain embodiments, R^{1d} is

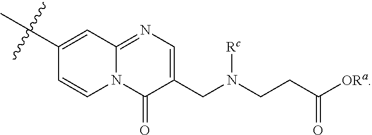

In certain embodiments, R^{1d} is

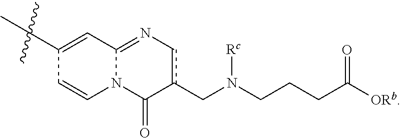

In certain embodiments, R^{1d} is

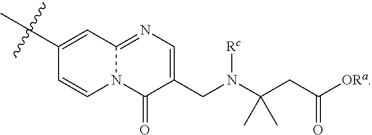

In certain embodiments, R^{1d} is

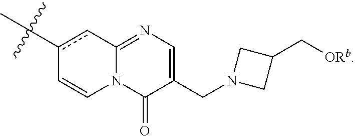

In certain embodiments, R^{1d} is

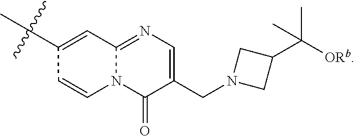

In certain embodiments, R^{1d} is

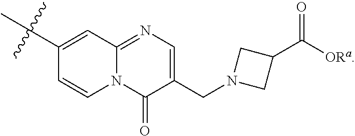

In certain embodiments, R^{1d} is

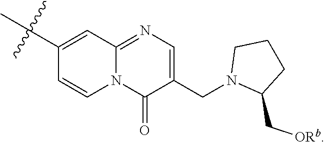

In certain embodiments, R^{1d} is

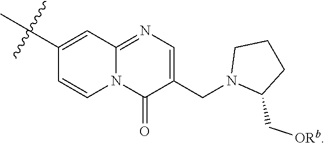

In certain embodiments, R^{1d} is

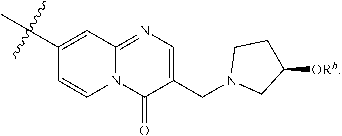

In certain embodiments, R^{1d} is

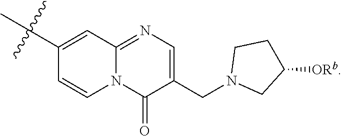

In certain embodiments, R^{1d} is

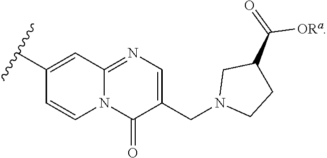

In certain embodiments, R^{1d} is

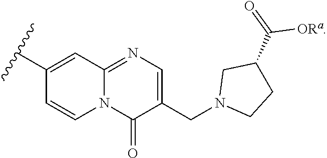

In certain embodiments, R^{1d} is

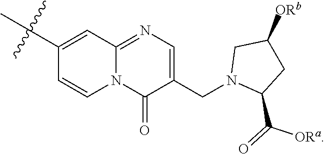

In certain embodiments, R^{1d} is

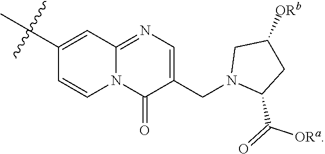

In certain embodiments, R^{1d} is

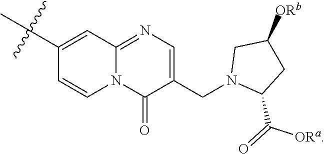

In certain embodiments, R^{1d} is

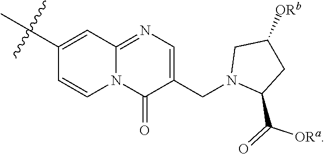

certain embodiments, R^{1d} is

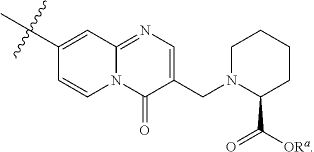

In certain embodiments, R^{1d} is

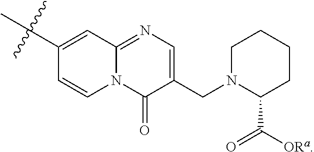

In certain embodiments, R^{1d} is

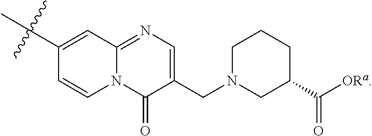

In certain embodiments, R^{1d} is

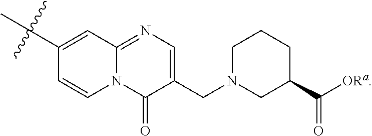

In certain embodiments, R^{1d} is

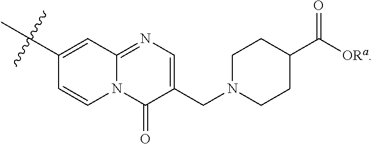

In certain embodiments, R^{1d} is

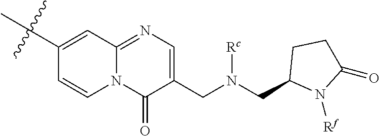

In certain embodiments, R^{1d} is

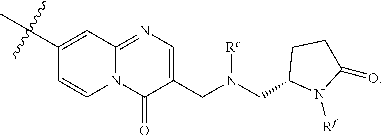

In certain embodiments, R^{1d} is

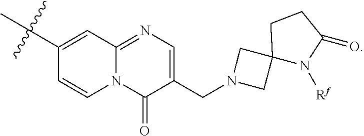

In certain embodiments, R^{1d} is

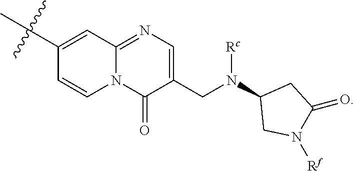

In certain embodiments, R^{1d} is

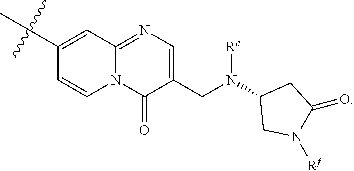

In certain embodiments, R^{1d} is

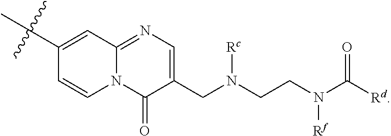

In certain embodiments R^{1d} is

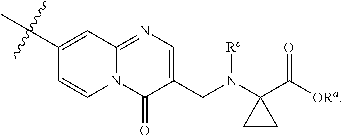

In certain embodiments, R^{1d} is

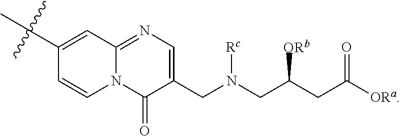

In certain embodiments, R^{1d} is

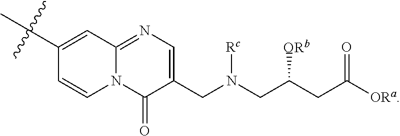

In certain embodiments, R^{1d} is

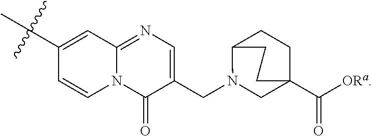

In certain embodiments, R^{1d} is

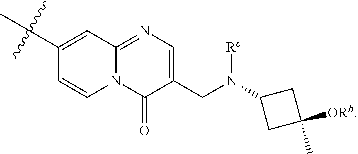

In certain embodiments, R^{1d} is

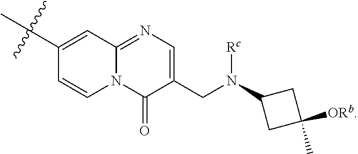

In certain embodiments, R^{1d} is

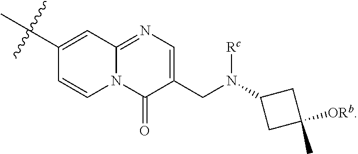

In certain embodiments, R^{1d} is

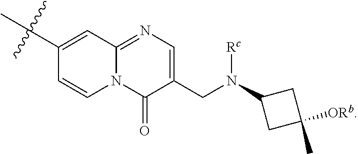

In certain embodiments, R^{1d} is

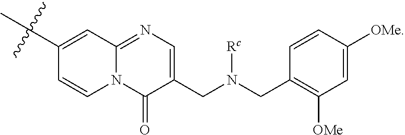

In certain embodiments, R^{1d} is

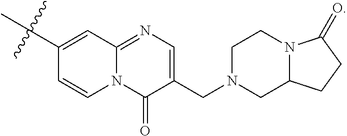 [SMILES: *c1ccn2c(=O)c(CN3CCN4C(=O)CCC4C3)cnc2c1]

In certain embodiments, R^{1d} is

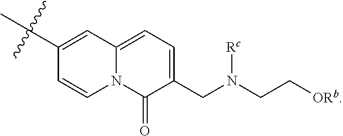

In certain embodiments, R^{1d} is

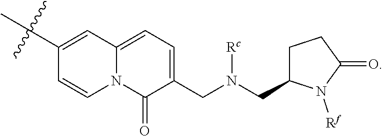

In certain embodiments, R^{1d} is

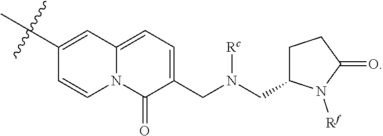

In certain embodiments, R^{1d} is

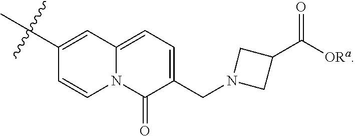

In certain embodiments, R^{1d} is

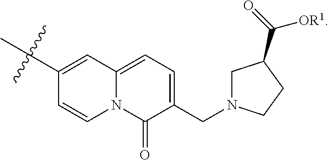

In certain embodiments, R^{1d} is

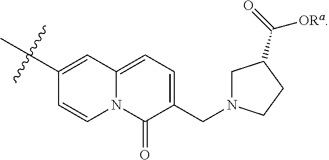

In certain embodiments, R^{1d} is

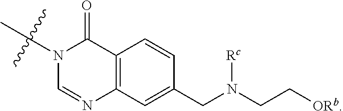

In certain embodiments, R^{1d} is

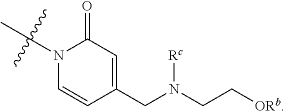

In certain embodiments, R^{1d} is

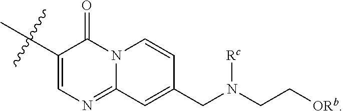

In certain embodiments, R^{1d} is

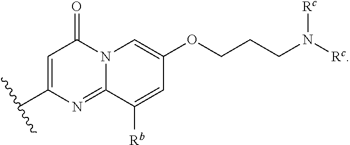

In certain embodiments, R^{1d} is

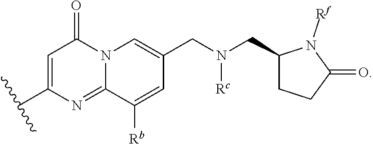

In certain embodiments, R^{1d} is

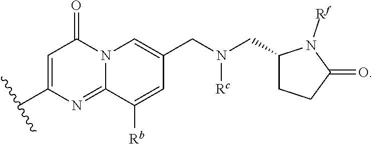

In certain embodiments, R^{1d} is

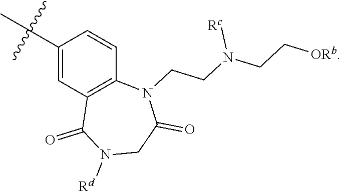

In certain embodiments, R^{1d} is

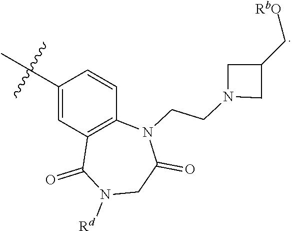

In certain embodiments, R^{1d} is

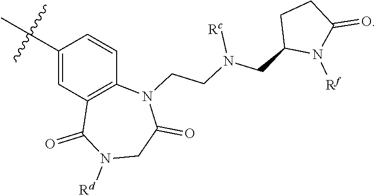

In certain embodiments, R^{1d} is

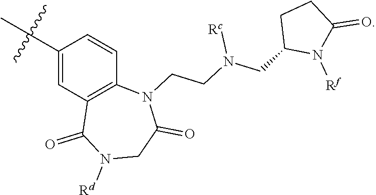

In certain embodiments, R^{1d} is

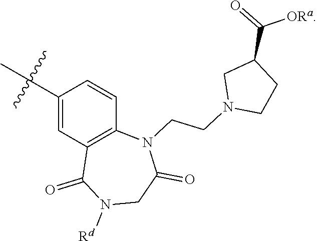

In certain embodiments, R^{1d} is

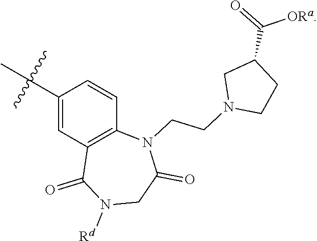

In certain embodiments, R^{1d} is

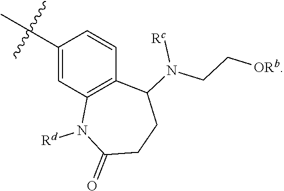

In certain embodiments R^{1d} is

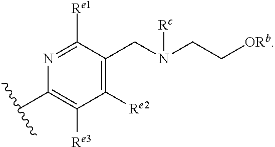

In certain embodiments, R^{1d} is

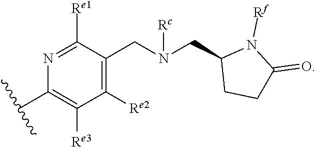

In certain embodiments, R^{1d} is

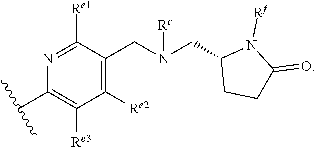

In certain embodiments, R^{1d} is

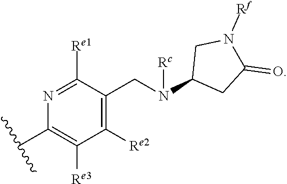

In certain embodiments, R^{1d} is

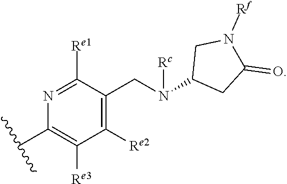

In certain embodiments, R^{1d} is

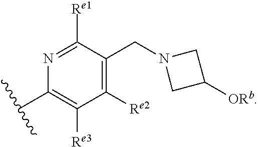

In certain embodiments, R^{1d} is

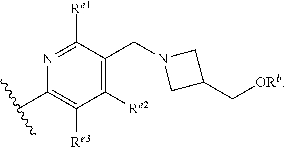

In certain embodiments, R^{1d} is

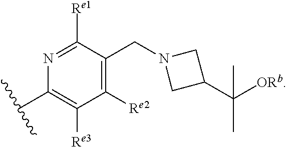

In certain embodiments, R^{1d} is

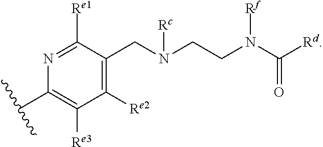

In certain embodiments, R^{1d} is

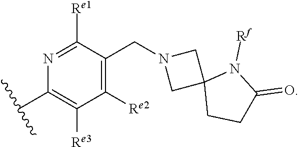

### In

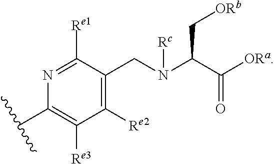

In certain embodiments, R^{1d} is

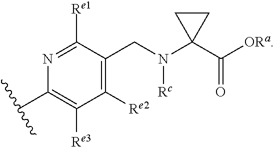

In certain embodiments, R^{1d} is

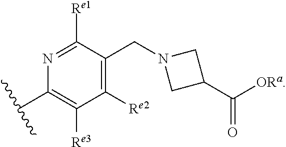

In certain embodiments, R^{1d} is

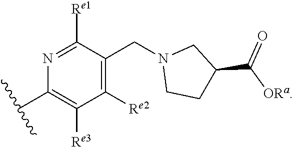

In certain embodiments, R^{1d} is

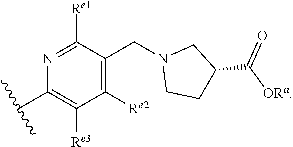

In certain embodiments, R^{1d} is

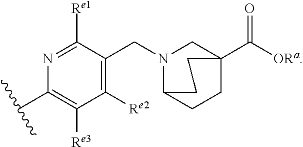

In certain embodiments, R^{1d} is

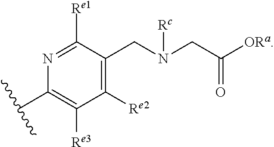

In certain embodiments, R^{1d} is

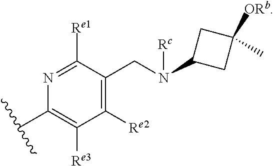

In certain embodiments, R^{1d} is

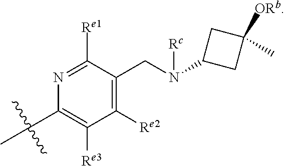

In certain embodiments, R^{1d} is

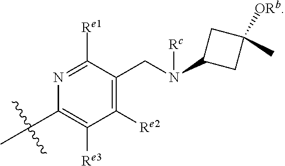

In certain embodiments, R^{1d} is

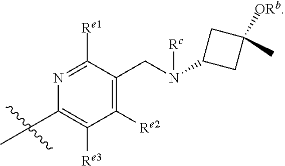

In certain embodiments, R^{1d} is

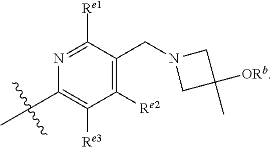

In certain embodiments, R^{1d} is

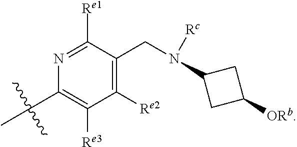

In certain embodiments, R^{1d} is

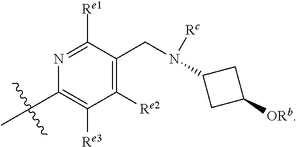

In certain embodiments, R^{1d} is

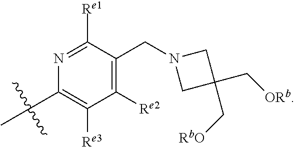

### In

certain embodiments, R^{1d} is

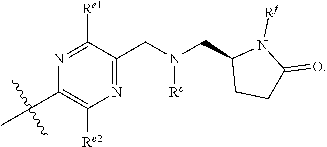

In certain embodiments, R^{1d} is

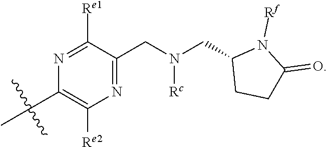

In certain embodiments, R^{1d} is

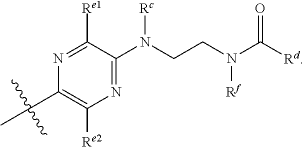

In certain embodiments, R^{1d} is

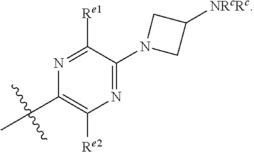

In certain embodiments, R^{1d} is

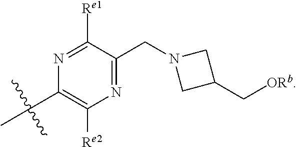

In certain embodiments, R^{1d} is

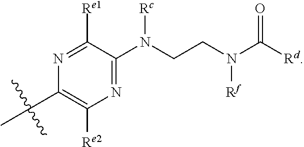

In certain embodiments, R^{1d} is

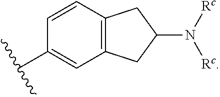

In certain embodiments, R^{1d} is

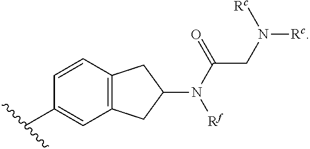

In certain embodiments, R^{1d} is

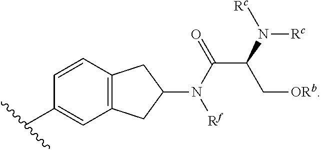

In certain embodiments, R^{1d} is

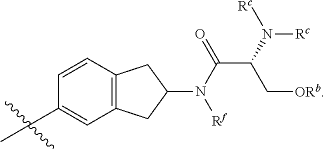

In certain embodiments, R is

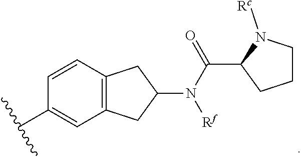

In certain embodiments, R^{1d} is

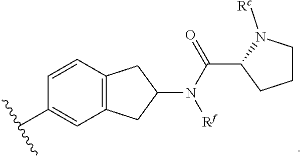

In certain embodiments, R^{1d} is

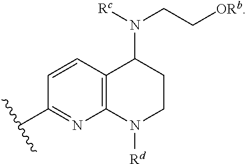

In certain embodiments, R^{1d} is

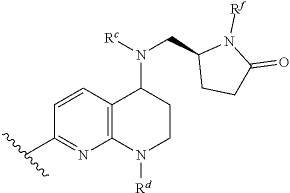

In certain embodiments, R^{1d} is

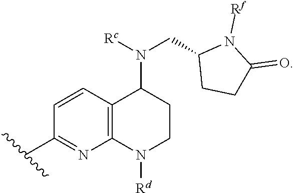

In certain embodiments, R^{1d} is

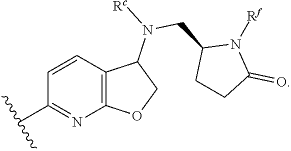

In certain embodiments, R^{1d} is

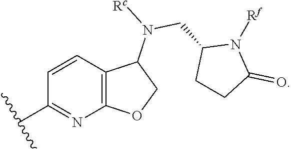

In certain embodiments, R^{1d} is

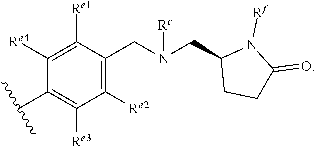

In certain embodiments, R^{1d} is

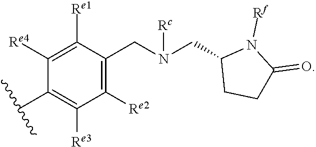

In certain embodiments, R^{1d} is

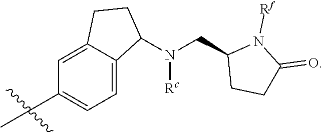

In certain embodiments, R is

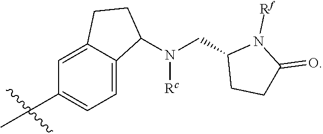

In certain embodiments, R^{1d} is

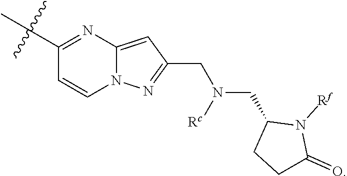

In certain embodiments, R^{1d} is

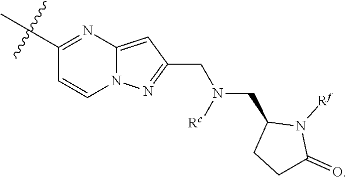

In certain embodiments, R^{1d} is

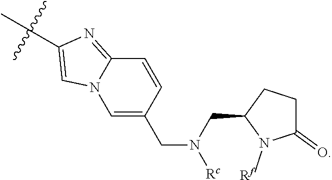

In certain embodiments, R^{1d} is

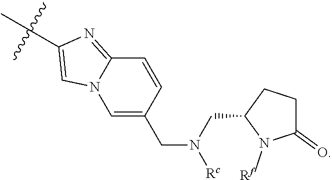

In certain embodiments, R^{1d} is

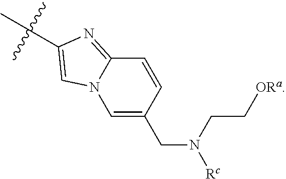

In certain embodiments, R^{1d} is

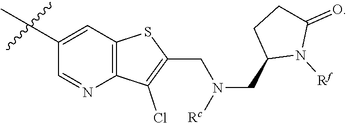

In certain embodiments, R^{1d} is

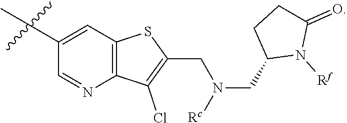

In certain embodiments, R^{1d} is

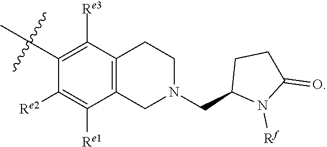

In certain embodiments, R^{1d} is

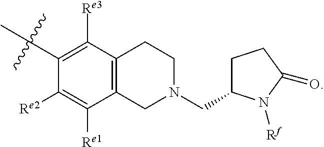

In certain embodiments, each occurrence of R^{a}, R^{b}, R^{c}, R^{d}, and R^{f} is independently selected from the group consisting of H, optionally substituted C₁-C₆ alkyl, and optionally substituted C₃-C₈ cycloalkyl. In certain embodiments, each occurrence of R^{e1}, R^{e2}, R^{e3}, and R^{e4} is independently selected from the group consisting of H, F, C₁, Br, C₁-C₆ alkyl, C₁-C₆ haloalkyl (such as but not limited to —CHF₂ and —CF₃), C₁-C₆ alkoxy, C₁-C₆ haloalkoxy (such as but not limited to —OCHF₂ and —OCF₃), C₁-C₆ thioalkoxy, C₃-C₈ cycloalkyl, C₃-C₈ halocycloalkyl, C₃-C₈ cycloalkoxy, C₃-C₈ halocycloalkoxy, C₃-C₈ thioalkoxy, and NR^{g}R^{g}, wherein each occurrence of R^{g} is independently H, optionally substituted C₁-C₆ alkyl, or optionally substituted C₃-C₈ cycloalkyl. In certain embodiments, at least one of R^{e1} and R^{e2} is not H. In certain embodiments, R^{e1} is not H. In certain embodiments, R^{e2} is not H.

In certain embodiments, at least one occurrence of R^{3a}/R^{3b} is —NR^{b}R^{c}. In certain 15 embodiments, at least one occurrence of R^{3a}/R^{3b} is —CH₂NR^{b}R^{c}. In certain embodiments, at least one occurrence of R^{3a}/R^{3b} is —CH₂N(Me)R^{c}. In certain embodiments, at least one occurrence of R^{3a}/R^{3b} is —CH₂NH₂. In certain embodiments, at least one occurrence of R^{3a}/R^{3b} is —CH₂NHMe. In certain embodiments, at least one occurrence of R^{3a}/R^{3b} is —CH₂NHEt. In certain embodiments, at least one occurrence of R^{3a}/R^{3b} is —CH₂NH(n-propyl). In certain embodiments, at least one occurrence of R^{3a}/R^{3b} is —CH₂NH(i-propyl). In certain embodiments, at least one occurrence of R^{3a}/R^{3b} is —CH₂NH(n-butyl). In certain embodiments, at least one occurrence of R^{3a}/R^{3b} is —CH₂NH(i-butyl). In certain embodiments, at least one occurrence of R^{3a}/R^{3b} is —CH₂NH(sec-butyl). In certain embodiments, at least one occurrence of R^{3a}/R^{3b} is —CH₂NH(t-butyl). In certain embodiments, at least one occurrence of R^{3a}/R^{3b} is —CH₂NH(n-pentyl). In certain embodiments, at least one occurrence of R^{3a}/R^{3b} is —CH₂NH(2-methylbutan-2-yl). In certain embodiments, at least one occurrence of R^{3a}/R^{3b} is —CH₂NH(2,2-dimethylpropyl). In certain embodiments, at least one occurrence of R^{3a}/R^{3b} is —CH₂NH(3-methylbutyl). In certain embodiments, at least one occurrence of R^{3a}/R^{3b} is —CH₂NH(pentan-2-yl). In certain embodiments, at least one occurrence of R^{3a}/R^{3b} is —CH₂NH(pentan-3-yl). In certain embodiments, at least one occurrence of R^{3a}/R^{3b} is —CH₂NH(3-methylbutan-2-yl). In certain embodiments, at least one occurrence of R^{3a}/R^{3b} is —CH₂NH(2-methylbutyl). In certain embodiments, at least one occurrence of R^{3a}/R^{3b} is —NR^{a}C(O)CH₂NR^{b}R^{c}. In certain embodiments, at least one occurrence of R^{3a}/R^{3b} is —NR^{a}C(O)CH(NR^{b}R^{c})CH₂OR^{d}. In certain embodiments, at least one occurrence of R^{3a}/R^{3b} is

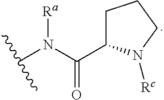

In certain embodiments, at least one occurrence of R^{3a}/R^{3b} is

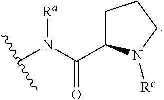

In certain embodiments, at least one occurrence of R^{3a}/R^{3b} is

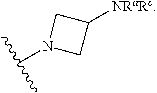

In certain embodiments, at least one occurrence of R^{3a}/R^{3b} is

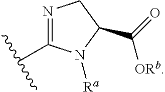

In certain embodiments, at least one occurrence of R^{3a}/R^{3b} is

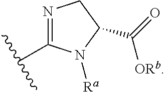

In certain embodiments, at least one occurrence of R^{3a}/R^{3b} is

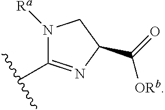

In certain embodiments, at least one occurrence of R^{3a}/R^{3b} is

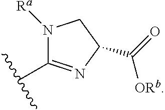

In certain embodiments, at least one occurrence of R^{3a}/R^{3b} is

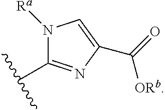

In certain embodiments, at least one occurrence of R^{3a}/R^{3b} is

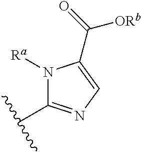

In certain embodiments, at least one occurrence of R^{3a}R3b is

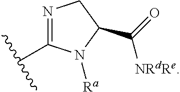

In certain embodiments, at least one occurrence of R^{3a}/R^{3b} is

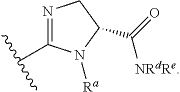

In certain embodiments, at least one occurrence of R^{3a}/R^{3b} is

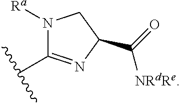

In certain embodiments, at least one occurrence of R^{3a}/R^{3b} is

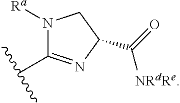

In certain embodiments, at least one occurrence of R^{3a}/R^{3b} is

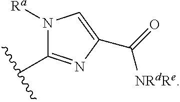

In certain embodiments, at least one occurrence of R^{3a}/R^{3b} is

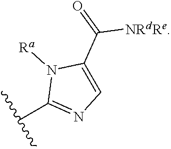

In certain embodiments, at least one occurrence of R^{3a}/R^{3b} is

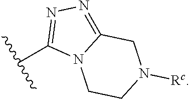

In certain embodiments, at least one occurrence of R^{3a}/R^{3b} is

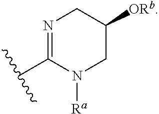

In certain embodiments, at least one occurrence of R^{3a}/R^{3b} is

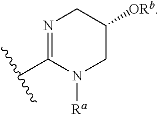

In certain embodiments, at least one occurrence of R^{3a}/R^{3b} is

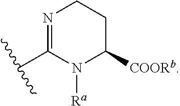

In certain embodiments, at least one occurrence of R^{3a}/R^{3b} is

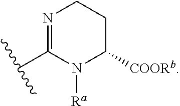

In certain embodiments, at least one occurrence of R^{3a}/R^{3b} is

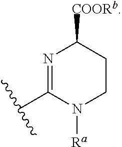

In certain embodiments, at least one occurrence of R^{3a}/R^{3b} is

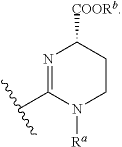

In certain embodiments, at least one occurrence of R^{3a}/R^{3b} is

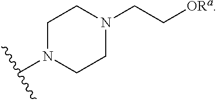

In certain embodiments, at least one occurrence of R^{3a}/R^{3b} is

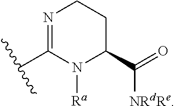

In certain embodiments, at least one occurrence of R^{3a}/R^{3b} is

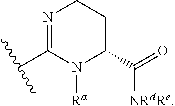

In certain embodiments, at least one occurrence of R^{3a}/R^{3b} is

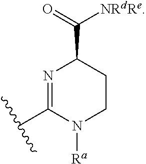

In certain embodiments, at least one occurrence of R^{3a}/R^{3b} is

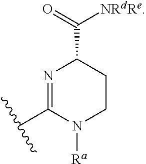

In certain embodiments, at least one occurrence of R^{3a}/R^{3b} is

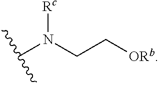

In certain embodiments, at least one occurrence of R^{3a}/R^{3b} is

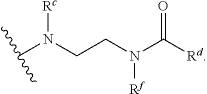

In certain embodiments, at least one occurrence of R^{3a}/R^{3b} is

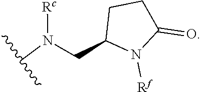

In certain embodiments, at least one occurrence of R^{3a}/R^{3b} is

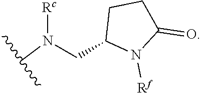

In certain embodiments, at least one occurrence of R^{3a}/R^{3b} is

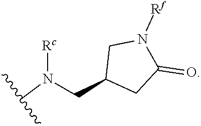

In certain embodiments, at least one occurrence of R^{3a}/R^{3b} is

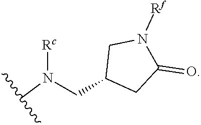

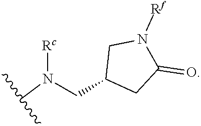

In certain embodiments, at least one occurrence of R^{3a}/R^{3b} is

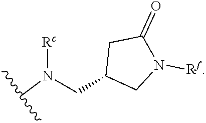

In certain embodiments, at least one occurrence of R^{3a}/R^{3b} is

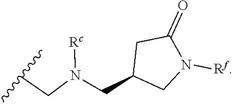

In certain embodiments, at least one occurrence of R^{3a}/R^{3b} is

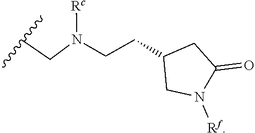

In certain embodiments, at least one occurrence of R^{3a}/R^{3b} is

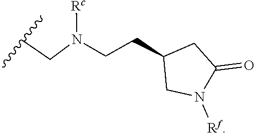

In certain embodiments, at least one occurrence of R^{3a}/R^{3b} is

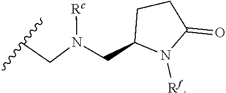

In certain embodiments, at least one occurrence of R^{3a}/R^{3b} is

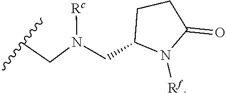

In certain embodiments, at least one occurrence of R^{3a}/R^{3b} is

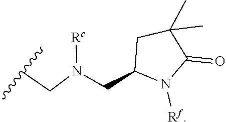

In certain embodiments, at least one occurrence of R^{3a}/R^{3b} is

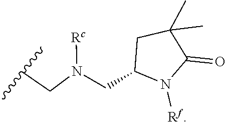

In certain embodiments, at least one occurrence of R^{3a}/R^{3b} is

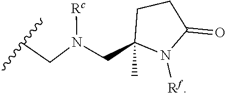

In certain embodiments, at least one occurrence of R^{3a}/R^{3b} is

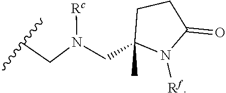

In certain embodiments, at least one occurrence of R^{3a}/R^{3b} is

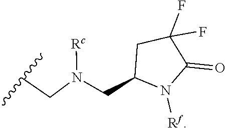

In certain embodiments, at least one occurrence of R^{3a}/R^{3b} is

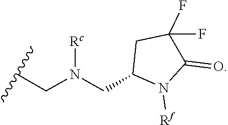

In certain embodiments, at least one occurrence of R^{3a}/R^{3b} is

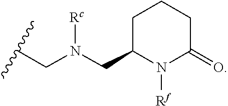

In certain embodiments, at least one occurrence of R^{3a}/R^{3b} is

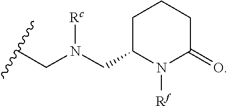

In certain embodiments, at least one occurrence of R^{3a}/R^{3b} is

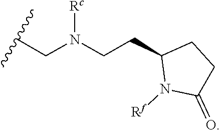

In certain embodiments, at least one occurrence of R^{3a}/R^{3b} is

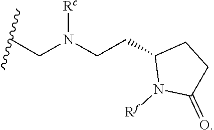

In certain embodiments, at least one occurrence of R^{3a}/R^{3b} is

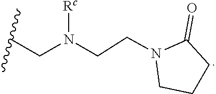

In certain embodiments, at least one occurrence of R^{3a}/R^{3b} is

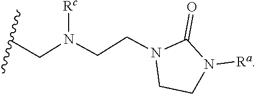

In certain embodiments, at least one occurrence of R^{3a}/R^{3b} is

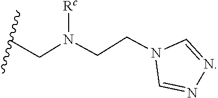

In certain embodiments, at least one occurrence of R^{3a}/R^{3b} is

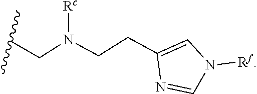

In certain embodiments, at least one occurrence of R^{3a}/R^{3b} is

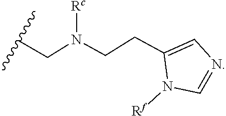

In certain embodiments, at least one occurrence of R^{3a}/R^{3b} is

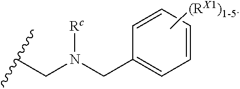

In certain embodiments, at least one occurrence of R^{3a}/R^{3b} is

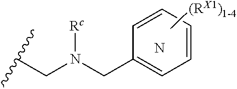

(wherein one of the ring atoms is N, or pyridyl). In certain embodiments, at least one occurrence of R^{3a}/R^{3b} is

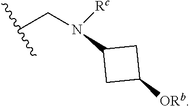

In certain embodiments, at least one occurrence of R^{3a}/R^{3b} is

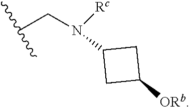

In certain embodiments, at least one occurrence of R^{3a}/R^{3b} is

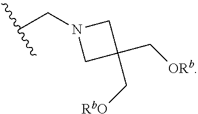

In certain embodiments, at least one occurrence of R^{3a}/R^{3b} is

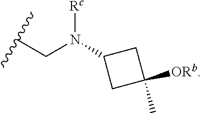

In certain embodiments, at least one occurrence of R^{3a}/R^{3b} is

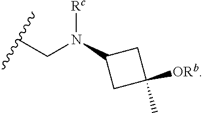

In certain embodiments, at least one occurrence of R^{3a}/R^{3b} is

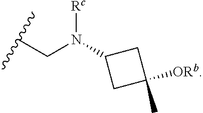

In certain embodiments, at least one occurrence of R^{3a}/R^{3b} is

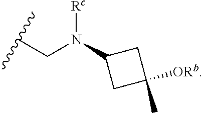

In certain embodiments, at least one occurrence of R^{3a}/R^{3b} is

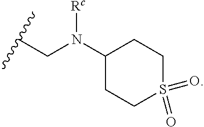

In certain embodiments, at least one occurrence of R^{3a}/R^{3b} is

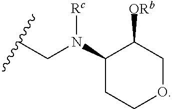

In certain embodiments, at least one occurrence of R^{3a}/R^{3b} is

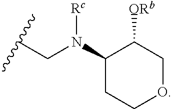

In certain embodiments, at least one occurrence of R^{3a}/R^{3b} is

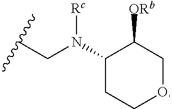

In certain embodiments, at least one occurrence of R^{3a}/R^{3b} is

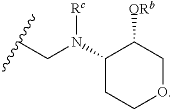

In certain embodiments, at least one occurrence of R^{3a}/R^{3b} is

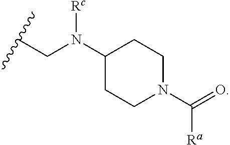

In certain embodiments, at least one occurrence of R^{3a}/R^{3b} is

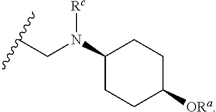

In certain embodiments, at least one occurrence of R^{3a}/R^{3b} is

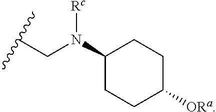

In certain embodiments, at least one occurrence of R^{3a}/R^{3b} is

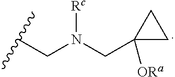

In certain embodiments, at least one occurrence of R^{3a}/R^{3b} is

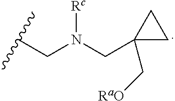

certain embodiments, at least one occurrence of R^{3a}/R^{b} is

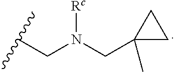

In certain embodiments, at least one occurrence of R^{3a}/R^{3b} is

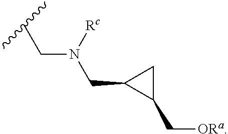

In certain embodiments, at least one occurrence of R^{3a}/R^{3b} is

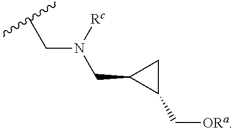

In certain embodiments, at least one occurrence of R^{3a}/R^{3b} is

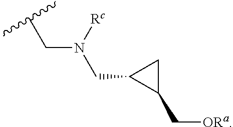

In certain embodiments, at least one occurrence of R^{3a}/R^{3b} is

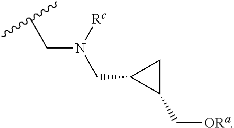

In certain embodiments, at least one occurrence of R^{3a}/R^{3b} is

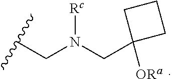

In certain embodiments, at least one occurrence of R^{3a}/R^{3b} is

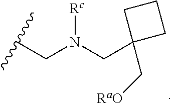

In certain embodiments, at least one occurrence of R^{3a}/R^{3b} is

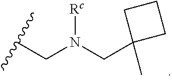

In certain embodiments, at least one occurrence of R^{3a}/R^{3b} is

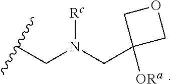

In certain embodiments, at least one occurrence of R^{3a}/R^{3b} is

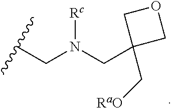

In certain embodiments, at least one occurrence of R^{3a}/R^{3b} is

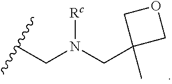

In certain embodiments, at least one occurrence of R^{3a}/R^{3b} is

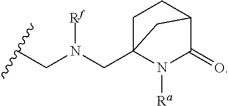

In certain embodiments, at least one occurrence of R^{3a}/R^{3b} is

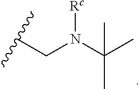

In certain embodiments, at least one occurrence of R^{3a}/R^{3b} is

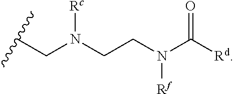

In certain embodiments, at least one occurrence of R^{3a}/R^{3b} is

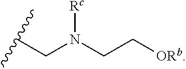

In certain embodiments, at least one occurrence of

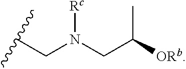

In certain embodiments, at least one occurrence of R^{3a}/R^{3b} is

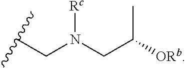

In certain embodiments, at least one occurrence of R^{3a}/R^{3b} is

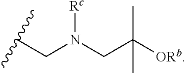

In certain embodiments, at least one occurrence of R^{3a}/R^{3b} is

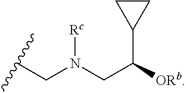

In certain embodiments, at least one occurrence of R^{3a}/R^{3b} is

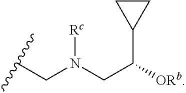

In certain embodiments, at least one occurrence of R^{3a}/R^{3b} is

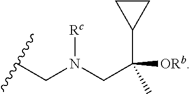

In certain embodiments, at least one occurrence of R^{3a}/R3b is

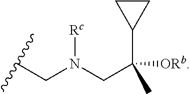

In certain embodiments, at least one occurrence of R^{3a}/R^{3b} is

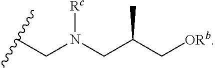

In certain embodiments, at least one occurrence of R^{3a}/R^{3b} is

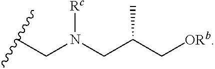

In certain embodiments, at least one occurrence of R^{3a}/R^{3b} is

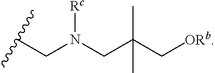

In certain embodiments, at least one occurrence of R^{3a}/R^{3b} is

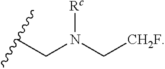

In certain embodiments, at least one occurrence of R^{3a}/R^{3b} is

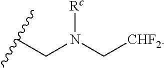

In certain embodiments, at least one occurrence of R^{3a}/R^{3b} is

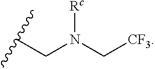

In certain embodiments, at least one occurrence of R^{3a}/R^{3b} is

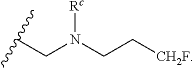

In certain embodiments, at least one occurrence of R^{3a}/R^{3b} is

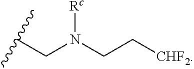

In certain embodiments, at least one occurrence of R^{3a}/R^{3b} is

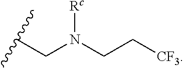

In certain embodiments, at least one occurrence of R^{3a}/R^{3b} is

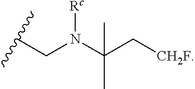

In certain embodiments, at least one occurrence of R^{3a}/R^{3b} is

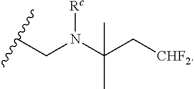

In certain embodiments, at least one occurrence of R^{3a}/R^{3b} is

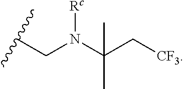

In certain embodiments, at least one occurrence of R^{3a}/R^{3b} is

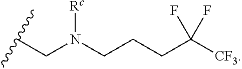

In certain embodiments, at least one occurrence of R^{3a}/R^{3b} is

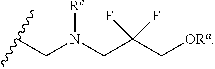

In certain embodiments, at least one occurrence of R^{3a}/R^{3b} is

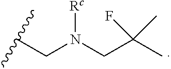

In certain embodiments, at least one occurrence of R^{3a}/R^{3b} is

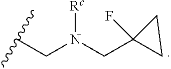

In certain embodiments, at least one occurrence of R^{3a}/R^{3b} is

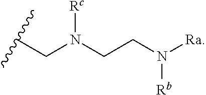

In certain embodiments, at least one occurrence of R^{3a}/R^{3b} is

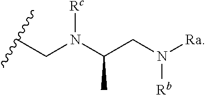

In certain embodiments, at least one occurrence of R^{3a}/R^{3b} is

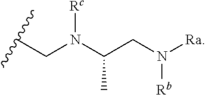

In certain embodiments, at least one occurrence of R^{3a}/R^{3b} is

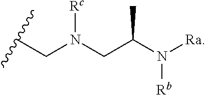

In certain embodiments, at least one occurrence of R^{3a}/R^{3b} is

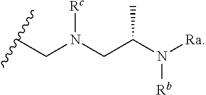

In certain embodiments, at least one occurrence of R^{3a}/R^{3b} is

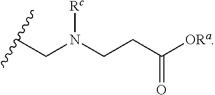

In certain embodiments, at least one occurrence of R^{3a}/R^{3b} is

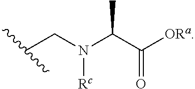

In certain embodiments, at least one occurrence of R^{3a}/R^{3b} is

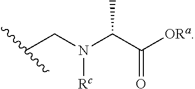

In certain embodiments, at least one occurrence of R^{3a}/R^{3b} is

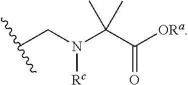

In certain embodiments, at least one occurrence of R^{3a}/R^{3b} is

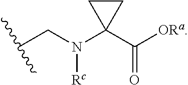

In certain embodiments, at least one occurrence of R^{3a}/R^{3b} is

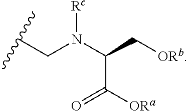

In certain embodiments, at least one occurrence of R^{3a}/R^{3b} is

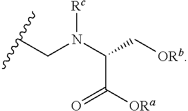

In certain embodiments, at least one occurrence of R³R^{3b} is

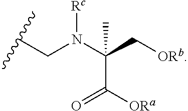

In certain embodiments, at least one occurrence of R^{3a}/R^{3b} is

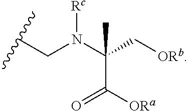

In certain embodiments, at least one occurrence of R^{3a}/R^{3b} is

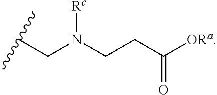

In certain embodiments, at least one occurrence of R^{3a}/R^{3b} is

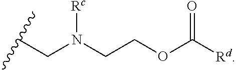

In certain embodiments, at least one occurrence of R^{3a}/R^{3b} is

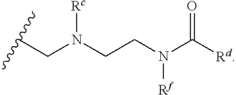

In certain embodiments, at least one occurrence of R^{3a}/R^{3b} is

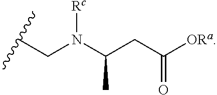

In certain embodiments, at least one occurrence of R^{3a}/R^{3b} is

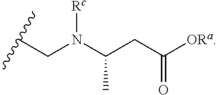

In certain embodiments, at least one occurrence of R^{3a}/R^{3b}

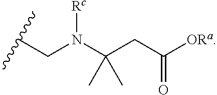

In certain embodiments, at least one occurrence of R^{3a}/R^{3b} is

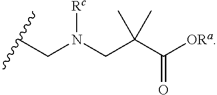

In certain embodiments, at least one occurrence of R^{3a}/R^{3b} is

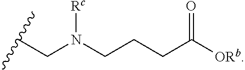

In certain embodiments, at least one occurrence of R^{3a}/R^{3b} is

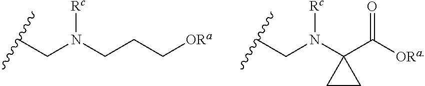

In certain embodiments, at least one occurrence of R^{3a}/R^{3b} is

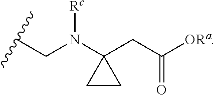

In certain embodiments, at least one occurrence of R^{3a}/R^{3b} is

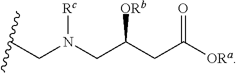

In certain embodiments, at least one occurrence of R^{3a}/R^{3b} is

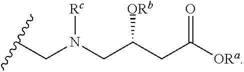

In certain embodiments, at least one occurrence of R^{3a}/R^{3b} is

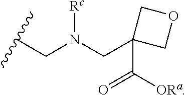

In certain embodiments, at least one occurrence of R^{3a}/R^{3b} is

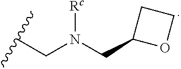

In certain embodiments, at least one occurrence of R^{3a}/R^{3b} is

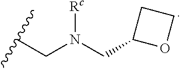

In certain embodiments, at least one occurrence of R^{3a}/R^{3b} is

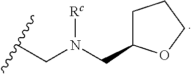

In certain embodiments, at least one occurrence of R^{3a}/R^{3b} is

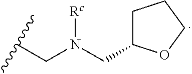

In certain embodiments, at least one occurrence of R^{3a}/R^{3b} is

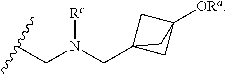

In certain embodiments, at least one occurrence of R^{3a}/R^{3b} is

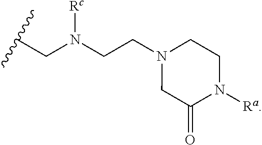

In certain embodiments, at least one occurrence of R^{3a}/R^{3b} is

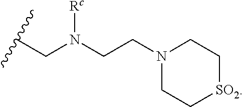

In certain embodiments, at least one occurrence of R^{3a}/R^{3b} is

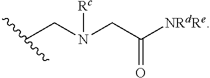

In certain embodiments, at least one occurrence of R^{3a}/R^{3b} is

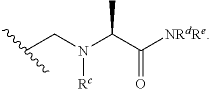

In certain embodiments, at least one occurrence of R^{3a}R^{3b}

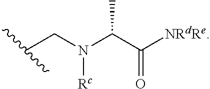

In certain embodiments, at least one occurrence of R^{3a}/R^{3b} is

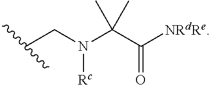

In certain embodiments, at least one occurrence of R^{3a}/R^{3b} is

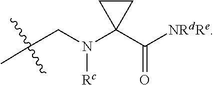

In certain embodiments, at least one occurrence of R^{3a}/R^{3b} is

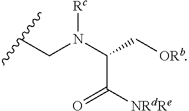

In certain

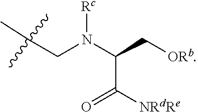

embodiments, at least one occurrence of R^{3a}/R^{3b} is
In certain embodiments, at least one occurrence of R^{3a}/R^{3b} is

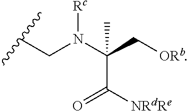

In certain

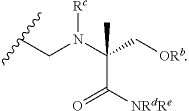

embodiments, at least one occurrence of R^{3a}/R^{3b} is
In certain embodiments, at least one occurrence of R^{3a}/R^{3b} is

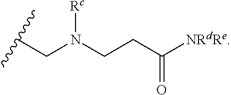

In certain embodiments, at least one occurrence of R^{3a}/R^{3b} is

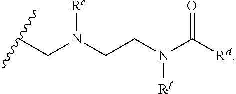

In certain embodiments, at least one occurrence of R^{3a}/R^{3b} is

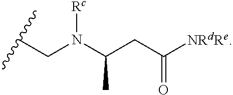

In certain embodiments, at least one occurrence of R^{3a}/R^{3b} is

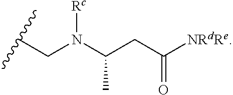

In certain embodiments, at least one occurrence of R^{3a}/R^{3b} is

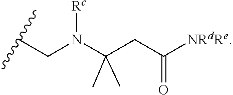

In certain embodiments, at least one occurrence of R^{3a}/R^{3b} is

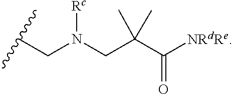

In certain embodiments, at least one occurrence of R^{3a}/R^{3b} is

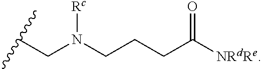

In certain embodiments, at least one occurrence of R^{3a}/R^{3b} is

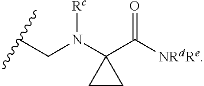

In certain embodiments, at least one occurrence
of R^{3a}/R^{3b} is

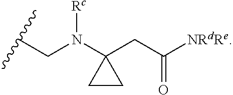

In certain embodiments, at least one occurrence of R^{3a}/R^{3b} is

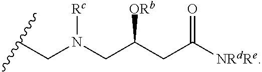

In certain embodiments, at least one occurrence of
R^{3a}/R^{3b} is

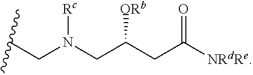

In certain embodiments, at least one occurrence of R^{3a}/R^{3b} is

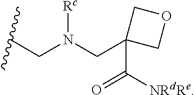

In certain embodiments, at least one occurrence of R^{3a}/R^{3b} is

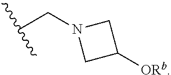

In certain embodiments, at least one occurrence of R^{3a}/R^{3b} is

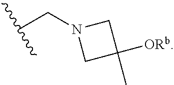

In certain embodiments, at least one occurrence of R^{3a}/R^{3b} is

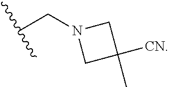 [SMILES: *CN1CC(C)(C#N)C1]

In certain embodiments, at least one occurrence of R^{3a}/R^{3b} is

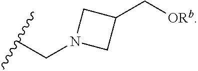

In certain embodiments, at least one occurrence of R^{3a}/R^{3b} is

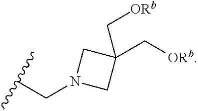

In certain embodiments, at least one occurrence of R^{3a}/R^{3b} is

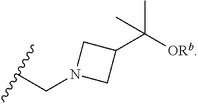

In certain embodiments, at least one occurrence of R^{3a}/R^{3b} is

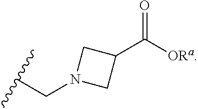

In certain embodiments, at least one occurrence of R^{3a}/R^{3b} is

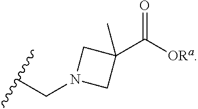

In certain embodiments, at least one occurrence of R^{3a}/R^{3b} is

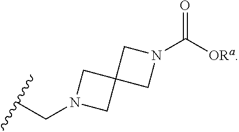

In certain embodiments, at least one occurrence of R^{3a}/R^{3b} is

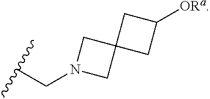

In certain embodiments, at least one occurrence of R^{3a}/R^{3b} is

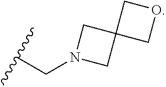 [SMILES: *CN1CC2(COC2)C1]

In certain embodiments, at least one occurrence of R^{3a}R^{3b} is

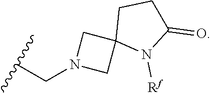

In certain embodiments, at least one occurrence of R^{3a}/R^{3b} is

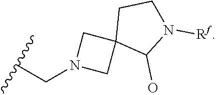

In certain embodiments, at least one occurrence of R^{3a}/R^{3b} is

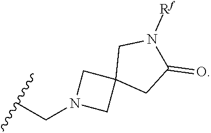

In certain embodiments, at least one occurrence of R^{3a}/R^{3b} is

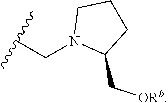

In certain embodiments, at least one occurrence of R^{3a}/R^{3b} is

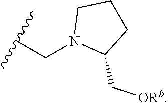

In certain embodiments, at least one occurrence of R^{3a}/R^{3b} is

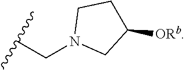

In certain embodiments, at least one occurrence of R^{3a}/R^{3b} is

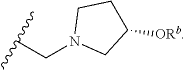

In certain embodiments, at least one occurrence of R^{3a}/R^{3b} is

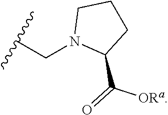

In certain embodiments, at least one occurrence of R^{3a}/R^{3b} is

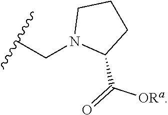

In certain embodiments, at least one occurrence of R^{3a}/R^{3b} is

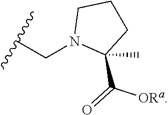

In certain embodiments, at least one occurrence of R^{3a}/R^{3b} is

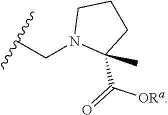

In certain embodiments, at least one occurrence of R^{3a}/R^{3b} is

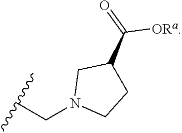

In certain embodiments, at least one occurrence of R^{3a}/R^{3b} is

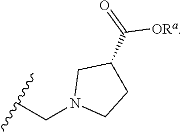

In certain embodiments, at least one occurrence of R^{3a}/R^{3b} is

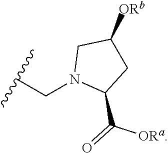

In certain embodiments, at least one occurrence of R^{3a}/R^{3b} is

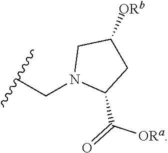

In certain embodiments, at least one occurrence of R^{3a}/R^{3b} is

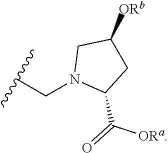

In certain embodiments, at least one occurrence of R^{3a}/R^{3b} is

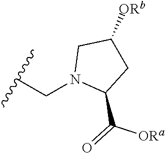

In certain embodiments, at least one occurrence of R^{3a}/R^{3b} is

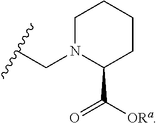

In certain embodiments, at least one occurrence of R^{3a}/R^{3b} is

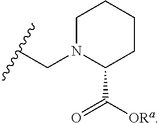

In certain embodiments, at least one occurrence of R^{3a}/R^{3b} is

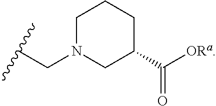

In certain embodiments, at least one occurrence of R^{3a}/R^{3b} is

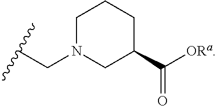

In certain embodiments, at least one occurrence of R^{3a}/R^{3b} is

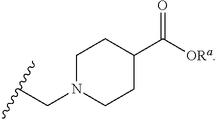

In certain embodiments, at least one occurrence of R^{3a}/R^{3b} is

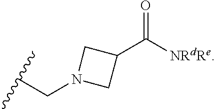

In certain embodiments, at least one occurrence of R^{3a}/R^{3b} is

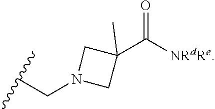

In certain embodiments, at least one occurrence of R^{3a}/R^{3b} is

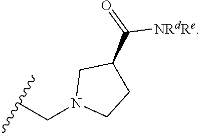

In certain embodiments, at least one occurrence of R^{3a}/R^{3b} is

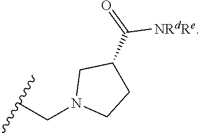

In certain embodiments, at least one occurrence of R^{3a}/R^{3b} is

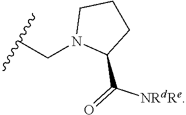

In certain embodiments, at least one occurrence of R^{3a}/R^{3b} is

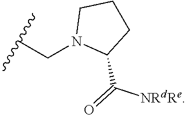

In certain embodiments, at least one occurrence of R^{3a}/R^{3b} is

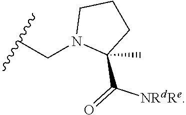

In certain embodiments, at least one occurrence of R^{3a}/R3b is

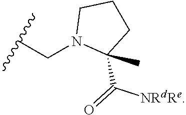

In certain embodiments, at least one occurrence of R^{3a}/R^{3b} is

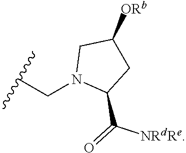

In certain embodiments, at least one occurrence of R^{3a}/R^{3b} is

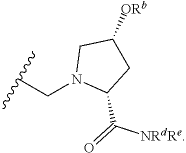

In certain embodiments, at least one occurrence of R^{3a}/R^{3b} is

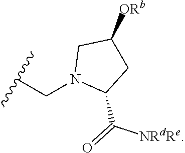

In certain embodiments, at least one occurrence of R^{3a}/R^{3b} is

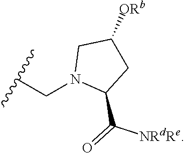

In certain embodiments, at least one occurrence of R^{3a}/R^{3b} is

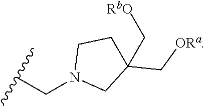

In certain embodiments, at least one occurrence of R^{3a}/R^{3b} is

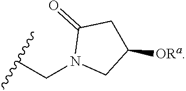

In certain embodiments, at least one occurrence of R^{3a}/R^{3b} is

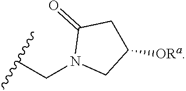

In certain embodiments, at least one occurrence of R^{3a}/R^{3b} is

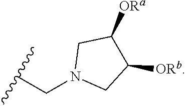

In certain embodiments, at least one occurrence of R^{3a}/R^{3b} is

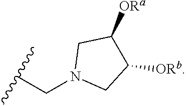

In certain embodiments, at least one occurrence of R^{3a}/R^{3b} is

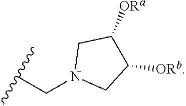

In certain embodiments, at least one occurrence of R^{3a}/R3b is

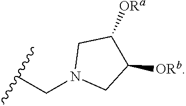

In certain embodiments, at least one occurrence of R^{3a}/R^{3b} is

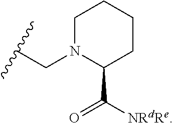

In certain embodiments, at least one occurrence of R^{3a}/R^{3b} is

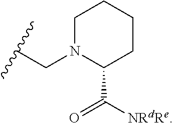

In certain embodiments, at least one occurrence of R^{3a}/R^{3b} is

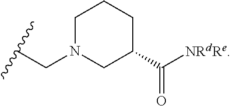

In certain embodiments, at least one occurrence of R^{3a}/R^{3b} is

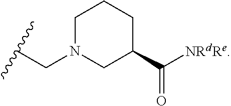

In certain embodiments, at least one occurrence of R^{3a}/R^{3b} is

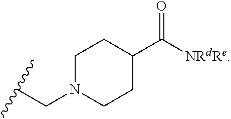

In certain embodiments, at least one occurrence of R^{3a}/R^{3b} is

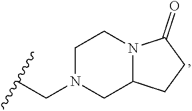 [SMILES: *CN1CCN2C(=O)CCC2C1]

In certain embodiments, at least one occurrence of R^{3a}/R^{3b} is

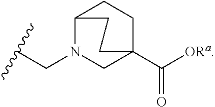

In certain embodiments, at least one occurrence of R^{3a}/R^{3b} is

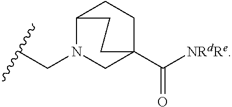

In certain embodiments, at least one occurrence of R^{3a}/R^{3b} is

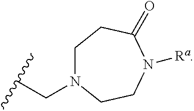

In certain embodiments, at least one occurrence of R^{3a}/R^{3b} is

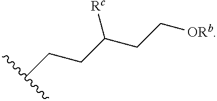

In certain embodiments, at least one occurrence of R^{3a}/R^{3b} is

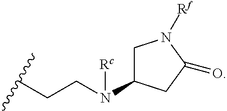

In certain embodiments, at least one occurrence of R^{3a}/R^{3b} is

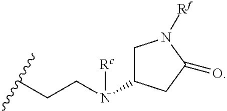

In certain embodiments, at least one occurrence of R^{3a}/R^{3b} is

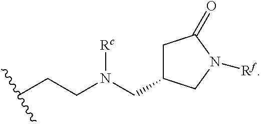

In certain embodiments, at least one occurrence of R^{3a}/R^{3b} is

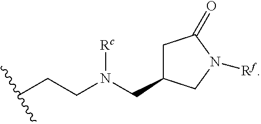

In certain embodiments, at least one occurrence of R^{3a}/R^{3b} is

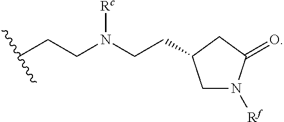

In certain embodiments, at least one occurrence of R^{3a}/R^{3b} is

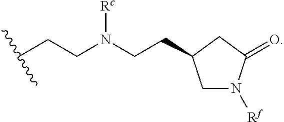

In certain embodiments, at least one occurrence of R^{3a}/R^{3b} is

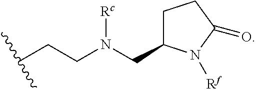

In certain embodiments, at least one occurrence of R^{3a}/R^{3b} is

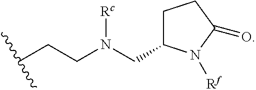

In certain embodiments, at least one occurrence of R^{3a}/R^{3b} is

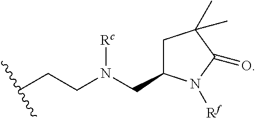

In certain embodiments, at least one occurrence of R^{3a}/R^{3b} is

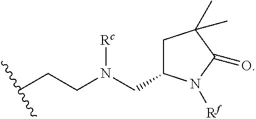

In certain embodiments, at least one occurrence of R^{3a}/R^{3b} is

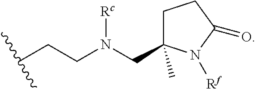

In certain embodiments, at least one occurrence of R^{3a}/R^{3b} is

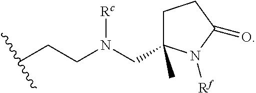

In certain embodiments, at least one occurrence of R^{3a}/R^{3b} is

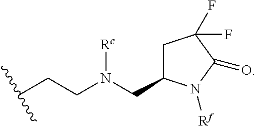

In certain embodiments, at least one occurrence of R^{3a}/R^{3b} is

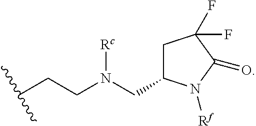

In certain embodiments, at least one occurrence of R^{3a}/R^{3b} is

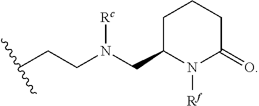

In certain embodiments, at least one occurrence of R^{3a}/R^{3b} is

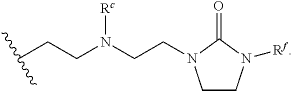

In certain embodiments, at least one occurrence of R^{3a}/R^{3b} is

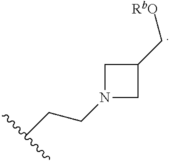

In certain embodiments, at least one occurrence of R^{3a}/R^{3b} is

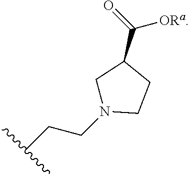

In certain embodiments, at least one occurrence of R^{3a}/R^{3b} is

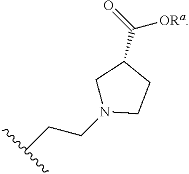

In certain embodiments, at least one occurrence of R^{3a}/R^{3b} is

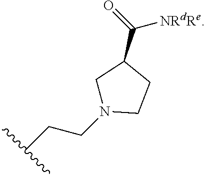

In certain embodiments, at least one occurrence of R^{3a}/R^{3b} is

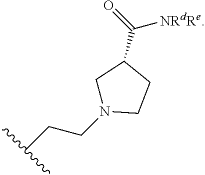

In certain embodiments, at least one occurrence of R^{3a}/R^{3b} is

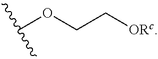

In certain embodiments, at least one occurrence of R^{3a}/R^{3b} is

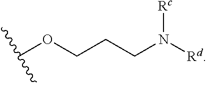

In certain embodiments, at least one occurrence of R^{3a}/R^{3b} is

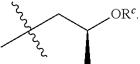

In certain embodiments, at least one occurrence of R^{3a}/R^{3b} is

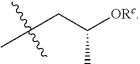

In certain embodiments, at least one occurrence of R^{3a}/R^{3b} is

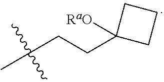

In certain embodiments, at least one occurrence of R^{3a}/R^{3b} is

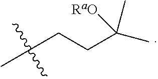

In certain embodiments, at least one occurrence of R^{3a}/R^{3b} is

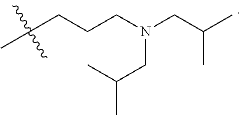 [SMILES: *CCCN(CC(C)C)CC(C)C]

In certain embodiments, at least one occurrence of R^{3a}/R^{3b} is

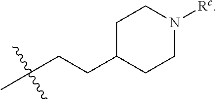

In certain embodiments, at least one occurrence of R^{3a}/R^{3b} is

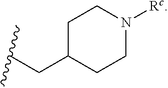

In certain embodiments, at least one occurrence of R^{3a}/R^{3b} is

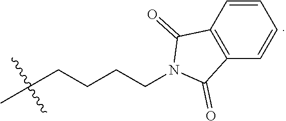 [SMILES: *CCCCN1C(=O)c2ccccc2C1=O]

In certain embodiments, at least one occurrence of R^{3a}/R^{3b} is

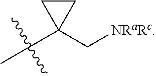

In certain embodiments, at least one occurrence of R^{3a}/R^{3b} is

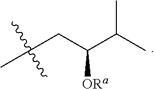

In certain embodiments, at least one occurrence of R^{3a}/R^{3b} is

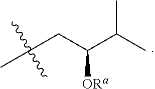

In certain embodiments, at least one occurrence of R^{3a}/R^{3b} is

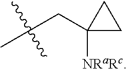

In certain embodiments, at least one occurrence of R^{3a}/R^{3b} is

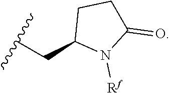

In certain embodiments, at least one occurrence of R^{3a}/R^{3b} is

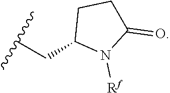

In certain embodiments, R^{2a} is H. In certain embodiments, R^{2a} is halogen. In certain embodiments, R^{2a} is C₁-C₆ alkyl. In certain embodiments, R^{2a} is C₁-C₆ alkoxy. In certain embodiments, R^{2a} is —(CH₂)₁₋₃(optionally substituted phenyl). In certain embodiments, R^{2a} is —(CH₂)₁₋₃(optionally substituted heteroaryl). In certain embodiments, R^{2a} is —O(CH₂)₁₋₃(optionally substituted phenyl). In certain embodiments, R^{2a} is —O(CH₂)₁₋₃(optionally substituted heteroaryl). In certain embodiments, R^{2a} is —CH₂)₁₋₃C(═O)OR¹. In certain embodiments, R^{2a} is —(CH₂)₁₋₃C(═O)NR^{I}R^{I}. In certain embodiments, R^{2a} is —O(CH₂)₁₋₃C(═O)OR¹. In certain embodiments, R^{2a} is —O(CH₂)₁₋₃C(═O)NR¹R1.

In certain embodiments, R^{2a} is selected from the group consisting of halogen, C₁-C₆ alkoxy, —CH₂(optionally substituted pyridinyl), —O(CH₂)₁₋₃C(═O)OH, and —O(CH₂)₁₋₃ C(═O)O(C₁-C₆ alkyl).

In certain embodiments, R^{3a} is —CHO. In certain embodiments, R^{3a} is —C(O)OR^{III}. In certain embodiments, R^{3a} is —C(═O)NR^{III}R^{III}. In certain embodiments, R^{3a} is —C(═NR⁵)NR^{III}R^{III}. In certain embodiments, R^{3a} is optionally substituted heterocyclyl. In certain embodiments, R^{3a} is —(CH₂)₁₋₃(optionally substituted heterocyclyl). In certain embodiments, R^{3a} is optionally substituted C₁-C₆ alkoxy. In certain embodiments, R^{3a} is optionally substituted C₁-C₆ aminoalkyl. In certain embodiments, R^{3a} is optionally substituted C₁-C₆ hydroxyalkyl.

In certain embodiments, R^{3a} is identical to R^{3b}. In other embodiments, R^{3a} is not identical to R^{3b}.

In certain embodiments, R^{3a} is selected from the group consisting of —CHO, —CH₂OH, —C(═NH)NH₂, —(CH₂)₀₋₁(optionally substituted piperidinyl), —(CH₂)₀₋₁(optionally substituted tetrahydropyrimidinyl), —(CH₂)₀₋₁(optionally substituted imidazolyl), —(CH₂)₀₋₁(optionally substituted dihydroimidazolyl), —C(═O)NH(C₁-C₆ hydroxyalkyl), —CH₂NH(C₁-C₆ haloalkyl), —CH₂NH(C₁-C₆ hydroxyalkyl), —CH₂N(C₁-C₆ hydroxyalkyl)(C₁-C₆ hydroxyalkyl), —CH₂NH(C₁-C₆ aminoalkyl), —CH₂NH(C₁-C₆ acetamidoalkyl), —CH₂NH—CH(COOH)(CH₂)₁₋₆ OH, —CH₂NH—CH(C(═O)OC₁-C₆alkyl)(CH₂)₁₋₆OH, —CH₂NH—C(C₁-C₆ alkyl)(COOH)(CH₂)₁₋₆OH, —CH₂NH—C(C₁-C₆ alkyl)(C(═O)OC₁-C₆alkyl)(CH₂)₁₋₆OH, and —CH₂N(C₀-C₆ alkyl)-(CH₂)₁₋₆-(optionally substituted 5-oxopyrrolidin-2-yl).

In certain embodiments, R^{3a} is selected from the group consisting of —C(═NH)NH₂, —(CH₂)₀₋₁(optionally substituted azetidinyl), —(CH₂)₀₋₁(optionally substituted pyrrolidinyl), —(CH₂)₀₋₁(optionally substituted piperidinyl), —(CH₂)₀₋₁(optionally substituted tetrahydropyrimidinyl), —(CH₂)₀₋₁(optionally substituted imidazolyl), and —(CH₂)₀₋₁(optionally substituted dihydroimidazolyl).

In certain embodiments, in R^{3a} the —C(═NH)NH₂, azetidinyl, pyrrolidinyl, piperidinyl, tetrahydropyrimidinyl, imidazolyl, or dihydroimidazolyl is optionally substituted with at least one selected from the group consisting of oxo (═O), C₁-C₆ alkyl, C₁-C₆ hydroxyalkyl, C₁-C₆ aminoalkyl, C₁-C₆N-acylaminoalkyl, —(CH₂)₀₋₃C(═O)OH, —(CH₂)₀₋₃C(═O)O(C₁-C₆ alkyl), —OH, C₁-C₆ alkoxy, —O(CH₂)₀₋₃C(═O)OH, or —O(CH₂)₀₋₃C(═O)O(C₁-C₆ alkyl).

In certain embodiments, R^{3a} is selected from the group consisting of: —CH₂CH₂OH, —CH₂NHCH₂CH₂OH, —CH₂CH₂NHCH₂CH₂OH, —CH₂NHCH(COOH)CH₂OH, —CH₂NHC(CH₃)(COOH)CH₂OH, —CH₂(2-carboxyazetidin-1-yl), —CH₂(3-carboxyazetidin-1-yl), —CH₂(2-carboxypyrrolidin-1-yl), —CH₂(3-carboxypyrrolidin-1-yl), —CH₂(2-carboxypiperidin-1-yl), —CH₂(3-carboxypiperidin-1-yl), —CH₂(4-carboxypiperidin-1-yl), and —CH₂NHCH₂-(5-oxopyrrolin-2-yl).

In certain embodiments, R^{3b} is —CHO. In certain embodiments, R^{3b} is —C(O)OR^{IV}. In certain embodiments, R^{3b} is —C(═O)NR^{IV}R^{IV}. In certain embodiments, R^{3b} is —C(═NR⁵)NR^{IV}R^{IV}. In certain embodiments, R^{3b} is optionally substituted heterocyclyl. In certain embodiments, R^{3b} is —(CH₂)₁₋₃(optionally substituted heterocyclyl). In certain embodiments, R^{3b} is optionally substituted C₁-C₆ alkoxy. In certain embodiments, R^{3b} is optionally substituted C₁-C₆ aminoalkyl. In certain embodiments, R^{3b} is optionally substituted C₁-C₆ hydroxyalkyl. In certain embodiments, R^{3b} is optionally substituted —(CH₂)₀₋₄(N-linked C₁-C₆ aminoalkyl). In certain embodiments, R^{3b} is optionally substituted —(CH₂)₀₋₄(N-linked azetidinyl). In certain embodiments, R^{3b} is optionally substituted —(CH₂)₀₋₄(N-linked pyrrolidinyl). In certain embodiments, R^{3b} is optionally substituted —(CH₂)₀₋₄(N-linked piperidinyl). In certain embodiments, R^{3b} is optionally substituted —(CH₂)₀₋₄(N-linked 2-azabicyclo[2.2.2]octyl). In certain embodiments, R^{3b} is optionally substituted —(CH₂)₀₋₄(N-linked 2,5-diazaspiro[3.4]octan-6-one). In certain embodiments, R^{3b} is optionally substituted —(CH₂)₀₋₄(N-linked hexahydropyrrolo[1,2-a]pyrazin-6(2H)-one). In certain embodiments, R^{3b} is optionally substituted —(CH₂)₀₋₄(O-linked C₁-C₆ alkyl). In certain embodiments, R^{3b} is optionally substituted —(CH₂)₀₋₄(pyrrolidin-2-one).

In certain embodiments, R^{3b} is selected from the group consisting of —CHO, —CH₂OH, —C(═NH)NH₂, —(CH₂)₀₋₁(optionally substituted piperidinyl), —(CH₂)₀₋₁(optionally substituted tetrahydropyrimidinyl), —(CH₂)₀₋₁(optionally substituted imidazolyl), —(CH₂)₀₋₁(optionally substituted dihydroimidazolyl), —C(═O)NH(C₁-C₆ hydroxyalkyl), —CH₂NH(C₁-C₆ haloalkyl), —CH₂NH(C₁-C₆ hydroxyalkyl), —CH₂N(C₁-C₆ hydroxyalkyl)(C₁-C₆ hydroxyalkyl), —CH₂NH(C₁-C₆ aminoalkyl), —CH₂NH(C₁-C₆ acetamidoalkyl), —CH₂NH—CH(COOH)(CH₂)₁₋₆ OH, —CH₂NH—CH(C(═O)OC₁-C₆alkyl)(CH₂)₁₋₆OH, —CH₂NH—C(C₁-C₆ alkyl)(COOH)(CH₂)₁₋₆ OH, —CH₂NH—C(C₁-C₆ alkyl)(C(═O)OC₁-C₆alkyl)(CH₂)₁₋₆OH, and —CH₂N(C₀-C₆ alkyl)-(CH₂)₁₋₆-(optionally substituted 5-oxopyrrolidin-2-yl).

In certain embodiments, R^{3b} is selected from the group consisting of —C(═NH)NH₂, —(CH₂)₀₋₁(optionally substituted azetidinyl), —(CH₂)₀₋₁(optionally substituted pyrrolidinyl), —(CH₂)₀₋₁(optionally substituted piperidinyl), —(CH₂)₀₋₁(optionally substituted tetrahydropyrimidinyl), —(CH₂)₀₋₁(optionally substituted imidazolyl), and —(CH₂)₀₋₁(optionally substituted dihydroimidazolyl).

In certain embodiments, in R^{3b} the —C(═NH)NH₂, azetidinyl, pyrrolidinyl, piperidinyl, tetrahydropyrimidinyl, imidazolyl, or dihydroimidazolyl is optionally substituted with at least one selected from the group consisting of oxo (═O), C₁-C₆ alkyl, C₁-C₆ hydroxyalkyl, C₁-C₆ aminoalkyl, C₁-C₆N-acylaminoalkyl, —(CH₂)₀₋₃C(═O)OH, —(CH₂)₁₋₃C(═O)O(C₁-C₆ alkyl), —OH, C₁-C₆ alkoxy, —O(CH₂)₀₋₃C(═O)OH, or —O(CH₂)₁₋₃C(═O)O(C₁-C₆ alkyl).

In certain embodiments, R^{3b} is selected from the group consisting of: —CH₂CH₂OH, —CH₂NHCH₂CH₂OH, —CH₂CH₂NHCH₂CH₂OH, —CH₂NHCH(COOH)CH₂OH, —CH₂NHC(CH₃)(COOH)CH₂OH, —CH₂(2-carboxyazetidin-1-yl), —CH₂(3-carboxyazetidin-1-yl), —CH₂(2-carboxypyrrolidin-1-yl), —CH₂(3-carboxypyrrolidin-1-yl), —CH₂(2-carboxypiperidin-1-yl), —CH₂(3-carboxypiperidin-1-yl), —CH₂(4-carboxypiperidin-1-yl), and —CH₂NHCH₂-(5-oxopyrrolin-2-yl).

In certain embodiments, R^{4a} is H. In yet other embodiments, R^{4a} is chloro.

In certain embodiments, the heterocyclyl is selected from the group consisting of azetidinyl, imidazolyl, dihydroimidazolyl, piperidinyl, piperazinyl, morpholinyl, pyrrolidinyl, pyrrolidin-2-one-yl, and tetrahydropyrimidinyl.

In certain embodiments, the heteroaryl is selected from the group consisting of pyridinyl, pyrimidinyl, pyrrolyl, oxazolyl, isoxazolyl, oxadiazolyl, imidazolyl, pyrazolyl, and triazolyl.

In certain embodiments, each occurrence of alkyl or cycloalkyl is independently optionally substituted with at least one substituent selected from the group consisting of C₁-C₆ alkyl, halo, —CN, —OR, phenyl (thus yielding, in non-limiting examples, optionally substituted phenyl-(C₁-C₃ alkyl), such as, but not limited to, benzyl or substituted benzyl), and —N(R)(R), wherein each occurrence of R is independently H, C₁-C₆ alkyl, or C₃-C₈ cycloalkyl.

In certain embodiments, each occurrence of phenyl, heterocyclyl, or heteroaryl is independently optionally substituted with at least one substituent selected from the group consisting of C₁-C₆ alkyl, C₁-C₆ haloalkyl, C₁-C₆ alkoxy, C₁-C₆ haloalkoxy, halo, —CN, —C(═O)OR, —OR, —N(R)(R), —NO₂, —S(═O)₂N(R)(R), acyl, and C₁-C₆ alkoxycarbonyl, wherein each occurrence of R is independently H, C₁-C₆ alkyl, or C₃-C₈ cycloalkyl.

In certain embodiments, the compound of formula (Ia) or (Ib) is selected from the group consisting of:
5-((4-chloro-2-(((2-hydroxyethyl)amino)methyl)-5-((3′-(3-(((2-hydroxyethyl)amino)methyl)-4-oxo-4H-pyrido[1,2-a]pyrimidin-8-yl)-2-methyl-[1,1′-biphenyl]-3-yl)methoxy)phenoxy)methyl)nicotinamide;
5-(((2-hydroxyethyl)amino)methyl)-N-(3′-(3-(((2-hydroxyethyl)amino)methyl)-4-oxo-4H-pyrido[1,2-a]pyrimidin-8-yl)-2-methyl-[1,1′-biphenyl]-3-yl)picolinamide;
5-((4-chloro-2-(((2-hydroxyethyl)amino)methyl)-5-((3′-(3-(((2-hydroxyethyl)amino)methyl)-4-oxo-4H-pyrido[1,2-a]pyrimidin-8-yl)-2,2′-dimethyl-[1,1′-biphenyl]-3-yl)methoxy)phenoxy)methyl)nicotinonitrile;
5-((4-chloro-2-(((2-hydroxyethyl)amino)methyl)-5-((3′-(3-(((2-hydroxyethyl)amino)methyl)-4-oxo-4H-pyrido[1,2-a]pyrimidin-8-yl)-2-methyl-[1,1′-biphenyl]-3-yl)methoxy)phenoxy)methyl)nicotinonitrile;
5-((4-chloro-2-(((2-hydroxyethyl)amino)methyl)-5-((3′-(7-(((2-hydroxyethyl)amino)methyl)-4-oxoquinazolin-3(4H)-yl)-2-methyl-[1,1′-biphenyl]-3-yl)methoxy)phenoxy)methyl)nicotinonitrile;
5-((4-chloro-2-(((2-hydroxyethyl)amino)methyl)-5-((3′-(7-(((2-hydroxyethyl)amino)methyl)-4-oxoquinazolin-3(4H)-yl)-2,2′-dimethyl-[1,1′-biphenyl]-3-yl)methoxy)phenoxy)methyl)nicotinonitrile;
(S)-2-(((8-(3′-((4-((((S)-2-carboxy-1-hydroxypropan-2-yl)amino)methyl)-2-chloro-5-((5-cyanopyridin-3-yl)methoxy)phenoxy)methyl)-2,2′-dimethyl-[1,1′-biphenyl]-3-yl)-4-oxo-4H-pyrido[1,2-a]pyrimidin-3-yl)methyl)amino)-3-hydroxy-2-methylpropanoic acid;
5-((4-chloro-5-((3′-(1-(2-((2-hydroxyethyl)amino)ethyl)-4-methyl-2,5-dioxo-2,3,4,5-tetrahydro-1H-benzo[e][1,4]diazepin-7-yl)-2,2′-dimethyl-[1,1′-biphenyl]-3-yl)methoxy)-2-(((2-hydroxyethyl)amino)methyl)phenoxy)methyl)nicotinonitrile;
5-((4-chloro-2-(((2-hydroxyethyl)amino)methyl)-5-((3′-(3-(((2-hydroxyethyl)amino)methyl)-4-oxo-4H-quinolizin-8-yl)-2,2′-dimethyl-[1,1′-biphenyl]-3-yl)methoxy)phenoxy)methyl)nicotinonitrile;
((8-(3′-((4-((((S)-1-carboxy-2-hydroxyethyl)amino)methyl)-2-chloro-5-((5-cyanopyridin-3-yl)methoxy)phenoxy)methyl)-2,2′-dimethyl-[1,1′-biphenyl]-3-yl)-4-oxo-4H-pyrido[1,2-a]pyrimidin-3-yl)methyl)-L-serine or(S)-2-(((8-(3′-((4-((((S)-2-carboxy-1-hydroxypropan-2-yl)amino)methyl)-2-chloro-5-((5-cyanopyridin-3-yl)methoxy)phenoxy)methyl)-2,2′-dimethyl-[1,1′-biphenyl]-3-yl)-4-oxo-4H-pyrido[1,2-a]pyrimidin-3-yl)methyl)amino)-3-hydroxy-2-methylpropanoic acid;
(S)-1-((8-(3′-((4-(((S)-2-carboxypiperidin-1-yl)methyl)-2-chloro-5-((5-cyanopyridin-3-yl)methoxy)phenoxy)methyl)-2,2′-dimethyl-[1,1′-biphenyl]-3-yl)-4-oxo-4H-pyrido[1,2-a]pyrimidin-3-yl)methyl)piperidine-2-carboxylic acid;
5-((4-chloro-5-((2,2′-dimethyl-3′-(4-oxo-3-(((((S)-5-oxopyrrolidin-2-yl)methyl)amino)methyl)-4H-pyrido[1,2-a]pyrimidin-8-yl)-[1,1′-biphenyl]-3-yl)methoxy)-2-(((((S)-5-oxopyrrolidin-2-yl)methyl)amino)methyl)phenoxy)methyl)nicotinonitrile;
5-((4-chloro-5-((2,2′-dimethyl-3′-(4-oxo-3-(((((R)-5-oxopyrrolidin-2-yl)methyl)amino)methyl)-4H-pyrido[1,2-a]pyrimidin-8-yl)-[1,1′-biphenyl]-3-yl)methoxy)-2-(((((R)-5-oxopyrrolidin-2-yl)methyl)amino)methyl)phenoxy)methyl)nicotinonitrile;
5-((4-chloro-5-((2-chloro-3′-(3-(((2-hydroxyethyl)amino)methyl)-4-oxo-4H-pyrido[1,2-a]pyrimidin-8-yl)-2′-methyl-[1,1′-biphenyl]-3-yl)methoxy)-2-(((2-hydroxyethyl)amino)methyl)phenoxy)methyl)nicotinonitrile;
8,8′-(2,2′-dimethyl-[1,1′-biphenyl]-3,3′-diyl)bis(3-(((2-hydroxyethyl)amino)methyl)-4H-pyrido[1,2-a]pyrimidin-4-one);
5-((4-chloro-5-((2-cyano-3′-(3-(((2-hydroxyethyl)amino)methyl)-4-oxo-4H-pyrido[1,2-a]pyrimidin-8-yl)-2′-methyl-[1,1′-biphenyl]-3-yl)methoxy)-2-(((2-hydroxyethyl)amino)methyl)phenoxy)methyl)nicotinonitrile;
5-((4-chloro-2-(((2-hydroxyethyl)amino)methyl)-5-((3′-(8-(((2-hydroxyethyl)amino)methyl)-4-oxo-4H-pyrido[1,2-a]pyrimidin-3-yl)-2,2′-dimethyl-[1,1′-biphenyl]-3-yl)methoxy)phenoxy)methyl)nicotinonitrile;
5-((4-chloro-2-(((2-hydroxyethyl)amino)methyl)-5-(((3′-(3-(((2-hydroxyethyl)amino)methyl)-4-oxo-4H-pyrido[1,2-a]pyrimidin-8-yl)-2,2′-dimethyl-[1,1′-biphenyl]-3-yl)oxy)methyl)phenoxy)methyl)nicotinonitrile;
5-((4-chloro-5-(((2,2′-dimethyl-3′-(4-oxo-3-(((((S)-5-oxopyrrolidin-2-yl)methyl)amino)methyl)-4H-pyrido[1,2-a]pyrimidin-8-yl)-[1,1′-biphenyl]-3-yl)oxy)methyl)-2-(((((S)-5-oxopyrrolidin-2-yl)methyl)amino)methyl)phenoxy)methyl)nicotinonitrile;
5-((4-chloro-5-((2,2′-dimethyl-3′-(4-methyl-2,5-dioxo-1-(2-((((S)-5-oxopyrrolidin-2-yl)methyl)amino)ethyl)-2,3,4,5-tetrahydro-1H-benzo[e][1,4]diazepin-7-yl)-[1,1′-biphenyl]-3-yl)methoxy)-2-(((((S)-5-oxopyrrolidin-2-yl)methyl)amino)methyl)phenoxy)methyl)nicotinonitrile;
(Z)-8-(3′-(2-fluoro-2-(5-(((2-hydroxyethyl)amino)methyl)pyridin-2-yl)vinyl)-2,2′-dimethyl-[1,1′-biphenyl]-3-yl)-3-(((2-hydroxyethyl)amino)methyl)-4H-pyrido[1,2-a]pyrimidin-4-one;
5-((4-chloro-5-((3′-(5-((2-hydroxyethyl)amino)-1-methyl-2-oxo-2,3,4,5-tetrahydro-1H-benzo[b]azepin-8-yl)-2,2′-dimethyl-[1,1′-biphenyl]-3-yl)methoxy)-2-(((2-hydroxyethyl)amino)methyl)phenoxy)methyl)nicotinonitrile;
5-((4-chloro-2-(((2-hydroxyethyl)amino)methyl)-5-((3′-(4-(((2-hydroxyethyl)amino)methyl)-2-oxopyridin-1(2H)-yl)-2,2′-dimethyl-[1,1′-biphenyl]-3-yl)methoxy)phenoxy)methyl)nicotinonitrile;
5-((4-chloro-5-((2,2′-dimethyl-3′-(4-oxo-3-(((((S)-5-oxopyrrolidin-2-yl)methyl)amino)methyl)-4H-quinolizin-8-yl)-[1,1′-biphenyl]-3-yl)methoxy)-2-(((((S)-5-oxopyrrolidin-2-yl)methyl)amino)methyl)phenoxy)methyl)nicotinonitrile;
5-((4-chloro-5-((2′-cyano-3′-(3-(((2-hydroxyethyl)amino)methyl)-4-oxo-4H-pyrido[1,2-a]pyrimidin-8-yl)-2-methyl-[1,1′-biphenyl]-3-yl)methoxy)-2-(((2-hydroxyethyl)amino)methyl)phenoxy)methyl)nicotinonitrile;
5-((4-chloro-5-((3′-(1-(2-(3-(hydroxymethyl)azetidin-1-yl)ethyl)-4-methyl-2,5-dioxo-2,3,4,5-tetrahydro-1H-benzo[e][1,4]diazepin-7-yl)-2,2′-dimethyl-[1,1′-biphenyl]-3-yl)methoxy)-2-((3-(hydroxymethyl)azetidin-1-yl)methyl)phenoxy)methyl)nicotinonitrile;
5-((4-chloro-5-(((2,2′-dimethyl-3′-(4-methyl-2,5-dioxo-1-(2-((((S)-5-oxopyrrolidin-2-yl)methyl)amino)ethyl)-2,3,4,5-tetrahydro-1H-benzo[e][1,4]diazepin-7-yl)-[1,1′-biphenyl]-3-yl)oxy)methyl)-2-(((((S)-5-oxopyrrolidin-2-yl)methyl)amino)methyl)phenoxy)methyl)nicotinonitrile;
5-((4-chloro-5-((2,2′-dimethyl-3′-(4-oxo-3-(((((R)-5-oxopyrrolidin-2-yl)methyl)amino)methyl)-4H-quinolizin-8-yl)-[1,1′-biphenyl]-3-yl)methoxy)-2-(((((R)-5-oxopyrrolidin-2-yl)methyl)amino)methyl)phenoxy)methyl)nicotinonitrile;
1-((8-(3′-((4-((3-carboxyazetidin-1-yl)methyl)-2-chloro-5-((5-cyanopyridin-3-yl)methoxy)phenoxy)methyl)-2,2′-dimethyl-[1,1′-biphenyl]-3-yl)-4-oxo-4H-quinolizin-3-yl)methyl)azetidine-3-carboxylic acid;
(S)-1-(4-((3′-(1-(2-((S)-3-carboxypyrrolidin-1-yl)ethyl)-4-methyl-2,5-dioxo-2,3,4,5-tetrahydro-1H-benzo[e][1,4]diazepin-7-yl)-2,2′-dimethyl-[1,1′-biphenyl]-3-yl)methoxy)-5-chloro-2-((5-cyanopyridin-3-yl)methoxy)benzyl)pyrrolidine-3-carboxylic acid;
8-(3′-(6-methoxy-5-(((((S)-5-oxopyrrolidin-2-yl)methyl)amino)methyl)pyridin-2-yl)-2,2′-dimethyl-[1,1′-biphenyl]-3-yl)-3-(((((S)-5-oxopyrrolidin-2-yl)methyl)amino)methyl)-4H-pyrido[1,2-a]pyrimidin-4-one;
8-(2′-chloro-3′-(6-methoxy-5-(((((S)-5-oxopyrrolidin-2-yl)methyl)amino)methyl)pyridin-2-yl)-2-methyl-[1,1′-biphenyl]-3-yl)-3-(((((S)-5-oxopyrrolidin-2-yl)methyl)amino)methyl)-4H-pyrido[1,2-a]pyrimidin-4-one;
8-(2,2′-dichloro-3′-(6-methoxy-5-(((((S)-5-oxopyrrolidin-2-yl)methyl)amino)methyl)pyridin-2-yl)-[1,1′-biphenyl]-3-yl)-3-(((((S)-5-oxopyrrolidin-2-yl)methyl)amino)methyl)-4H-pyrido[1,2-a]pyrimidin-4-one;
2,2′-(((2,2′-dichloro-[1,1′-biphenyl]-3,3′-diyl)bis(1-methyl-1,2,3,4-tetrahydro-1,8-naphthyridine-7,4-diyl))bis(azanediyl))bis(ethan-1-ol);
2,2′-(2,2′-dichloro-[1,1′-biphenyl]-3,3′-diyl)bis(9-methyl-7-(3-(methylamino)propoxy)-4H-pyrido[1,2-a]pyrimidin-4-one);
8-(2′-chloro-3′-(6-methoxy-5-(((((S)-5-oxopyrrolidin-2-yl)methyl)amino)methyl)pyrazin-2-yl)-2-methyl-[1,1′-biphenyl]-3-yl)-3-(((((S)-5-oxopyrrolidin-2-yl)methyl)amino)methyl)-4H-pyrido[1,2-a]pyrimidin-4-one;
8,8′-(2,2′-dimethyl-[1,1′-biphenyl]-3,3′-diyl)bis(3-(((((S)-5-oxopyrrolidin-2-yl)methyl)amino)methyl)-4H-pyrido[1,2-a]pyrimidin-4-one);
N,N′-((((2,2′-dichloro-[1,1′-biphenyl]-3,3′-diyl)bis(3-methoxypyrazine-5,2-diyl))bis(azanediyl))bis(ethane-2,1-diyl))diacetamide;
N,N′-((((2,2′-dichloro-[1,1′-biphenyl]-3,3′-diyl)bis(6-methoxypyrazine-5,2-diyl))bis(azanediyl))bis(ethane-2,1-diyl))diacetamide;
8-(3′-(2-amino-2,3-dihydro-1H-inden-5-yl)-2′-chloro-2-methyl-[1,1′-biphenyl]-3-yl)-3-(((((S)-5-oxopyrrolidin-2-yl)methyl)amino)methyl)-4H-pyrido[1,2-a]pyrimidin-4-one;
8-(3′-(2-amino-2,3-dihydro-1H-inden-5-yl)-2,2′-dimethyl-[1,1′-biphenyl]-3-yl)-3-(((((S)-5-oxopyrrolidin-2-yl)methyl)amino)methyl)-4H-pyrido[1,2-a]pyrimidin-4-one;
(5S,5'S)-5,5′-((((2,2′-dichloro-[1,1′-biphenyl]-3,3′-diyl)bis(1-methyl-1,2,3,4-tetrahydro-1,8-naphthyridine-7,4-diyl))bis(azanediyl))bis(methylene))bis(pyrrolidin-2-one);
2-amino-N-(5-(2-chloro-2′-methyl-3′-(4-oxo-3-(((((S)-5-oxopyrrolidin-2-yl)methyl)amino)methyl)-4H-pyrido[1,2-a]pyrimidin-8-yl)-[1,1′-biphenyl]-3-yl)-2,3-dihydro-1H-inden-2-yl)acetamide;
(5S,5'S)-5,5′-((((2,2′-dichloro-[1,1′-biphenyl]-3,3′-diyl)bis(2,3-dihydrofuro[2,3-b]pyridine-6,3-diyl))bis(azanediyl))bis(methylene))bis(pyrrolidin-2-one);
(2S)-2-amino-N-(5-(2-chloro-2′-methyl-3′-(4-oxo-3-(((((S)-5-oxopyrrolidin-2-yl)methyl)amino)methyl)-4H-pyrido[1,2-a]pyrimidin-8-yl)-[1,1′-biphenyl]-3-yl)-2,3-dihydro-1H-inden-2-yl)-3-hydroxypropanamide;
(2S)—N-(5-(2-chloro-2′-methyl-3′-(4-oxo-3-(((((S)-5-oxopyrrolidin-2-yl)methyl)amino)methyl)-4H-pyrido[1,2-a]pyrimidin-8-yl)-[1,1′-biphenyl]-3-yl)-2,3-dihydro-1H-inden-2-yl)pyrrolidine-2-carboxamide;
8-(2,2′-dichloro-3′-(6-methoxy-5-(((((S)-5-oxopyrrolidin-2-yl)methyl)amino)methyl)pyrazin-2-yl)-[1,1′-biphenyl]-3-yl)-3-(((((S)-5-oxopyrrolidin-2-yl)methyl)amino)methyl)-4H-pyrido[1,2-a]pyrimidin-4-one;
8,8′-(2,2′-dichloro-[1,1′-biphenyl]-3,3′-diyl)bis(3-(((((S)-5-oxopyrrolidin-2-yl)methyl)amino)methyl)-4H-pyrido[1,2-a]pyrimidin-4-one);
8-(2′-chloro-3′-(6-methoxy-5-(((((S)-5-oxopyrrolidin-2-yl)methyl)amino)methyl)pyrazin-2-yl)-2-methyl-[1,1′-biphenyl]-3-yl)-3-(((((S)-5-oxopyrrolidin-2-yl)methyl)amino)methyl)-4H-quinolizin-4-one;
8-(2′-chloro-3′-(6-chloro-5-(((((S)-5-oxopyrrolidin-2-yl)methyl)amino)methyl)pyridin-2-yl)-2-methyl-[1,1′-biphenyl]-3-yl)-3-(((((S)-5-oxopyrrolidin-2-yl)methyl)amino)methyl)-4H-pyrido[1,2-a]pyrimidin-4-one;
(5S)-5-(((7-(2,2′-dichloro-3′-(6-methoxy-5-(((((S)-5-oxopyrrolidin-2-yl)methyl)amino)methyl)pyridin-2-yl)-[1,1′-biphenyl]-3-yl)-1-methyl-1,2,3,4-tetrahydro-1,8-naphthyridin-4-yl)amino)methyl)pyrrolidin-2-one;
8-(2′-chloro-3′-(6-methoxy-5-(((((S)-5-oxopyrrolidin-2-yl)methyl)amino)methyl)pyridin-2-yl)-2-methyl-[1,1′-biphenyl]-3-yl)-3-((methyl(((S)-5-oxopyrrolidin-2-yl)methyl)amino)methyl)-4H-pyrido[1,2-a]pyrimidin-4-one;
(S)-8-(2′-chloro-3′-(5-((3-hydroxyazetidin-1-yl)methyl)-6-methoxypyridin-2-yl)-2-methyl-[1,1′-biphenyl]-3-yl)-3-((methyl((5-oxopyrrolidin-2-yl)methyl)amino)methyl)-4H-pyrido[1,2-a]pyrimidin-4-one;
(5S)-5-(((7-(2,2′-dichloro-3′-(6-methoxy-5-(((((S)-5-oxopyrrolidin-2-yl)methyl)amino)methyl)pyrazin-2-yl)-[1,1′-biphenyl]-3-yl)-1-methyl-1,2,3,4-tetrahydro-1,8-naphthyridin-4-yl)amino)methyl)pyrrolidin-2-one;
8-(2′-chloro-3′-(6-fluoro-5-(((((S)-5-oxopyrrolidin-2-yl)methyl)amino)methyl)pyridin-2-yl)-2-methyl-[1,1′-biphenyl]-3-yl)-3-(((((S)-5-oxopyrrolidin-2-yl)methyl)amino)methyl)-4H-pyrido[1,2-a]pyrimidin-4-one;
2-(2,2′-dichloro-3′-(6-methoxy-5-(((((S)-5-oxopyrrolidin-2-yl)methyl)amino)methyl)pyrazin-2-yl)-[1,1′-biphenyl]-3-yl)-7-(((((S)-5-oxopyrrolidin-2-yl)methyl)amino)methyl)-4H-pyrido[1,2-a]pyrimidin-4-one;
2-(2,2′-dichloro-3′-(6-methoxy-5-(((((S)-5-oxopyrrolidin-2-yl)methyl)amino)methyl)pyridin-2-yl)-[1,1′-biphenyl]-3-yl)-7-(((((S)-5-oxopyrrolidin-2-yl)methyl)amino)methyl)-4H-pyrido[1,2-a]pyrimidin-4-one;
2,2′-(2,2′-dichloro-[1,1′-biphenyl]-3,3′-diyl)bis(7-(((((S)-5-oxopyrrolidin-2-yl)methyl)amino)methyl)-4H-pyrido[1,2-a]pyrimidin-4-one);
8-(2′-chloro-2-methyl-3′-(5-(((((S)-5-oxopyrrolidin-2-yl)methyl)amino)methyl)pyridin-2-yl)-[1,1′-biphenyl]-3-yl)-3-(((((S)-5-oxopyrrolidin-2-yl)methyl)amino)methyl)-4H-pyrido[1,2-a]pyrimidin-4-one;
8-(2′-chloro-3′-(6-fluoro-5-(((((S)-5-oxopyrrolidin-2-yl)methyl)amino)methyl)pyridin-2-yl)-2-methyl-[1,1′-biphenyl]-3-yl)-3-(((5-oxopyrrolidin-3-yl)amino)methyl)-4H-pyrido[1,2-a]pyrimidin-4-one;
8-(2,2′-dichloro-3′-(6-methoxy-5-(((5-oxopyrrolidin-3-yl)amino)methyl)pyridin-2-yl)-[1,1′-biphenyl]-3-yl)-3-(((5-oxopyrrolidin-3-yl)amino)methyl)-4H-pyrido[1,2-a]pyrimidin-4-one;
8-(2′-chloro-3′-(6-(dimethylamino)-5-(((((S)-5-oxopyrrolidin-2-yl)methyl)amino)methyl)pyridin-2-yl)-2-methyl-[1,1′-biphenyl]-3-yl)-3-(((((S)-5-oxopyrrolidin-2-yl)methyl)amino)methyl)-4H-pyrido[1,2-a]pyrimidin-4-one;
8-(2′-chloro-2-methyl-4″-(((((S)-5-oxopyrrolidin-2-yl)methyl)amino)methyl)-[1,1′:3′,1″-terphenyl]-3-yl)-3-(((((S)-5-oxopyrrolidin-2-yl)methyl)amino)methyl)-4H-pyrido[1,2-a]pyrimidin-4-one;
8-(2′-chloro-3′-methoxy-2-methyl-4″-(((((S)-5-oxopyrrolidin-2-yl)methyl)amino)methyl)-[1,1′:3′,1″-terphenyl]-3-yl)-3-(((((S)-5-oxopyrrolidin-2-yl)methyl)amino)methyl)-4H-pyrido[1,2-a]pyrimidin-4-one;
8-(2′,3″-dichloro-2-methyl-4″-(((((S)-5-oxopyrrolidin-2-yl)methyl)amino)methyl)-[1,1′:3′,1″-terphenyl]-3-yl)-3-(((((S)-5-oxopyrrolidin-2-yl)methyl)amino)methyl)-4H-pyrido[1,2-a]pyrimidin-4-one;
8-(2′-chloro-2-methyl-3′-(8-methyl-5-((((S)-5-oxopyrrolidin-2-yl)methyl)amino)-5,6,7,8-tetrahydro-1,8-naphthyridin-2-yl)-[1,1′-biphenyl]-3-yl)-3-(((((S)-5-oxopyrrolidin-2-yl)methyl)amino)methyl)-4H-pyrido[1,2-a]pyrimidin-4-one;
N-(2-(((6-(3′-(3-(((2-acetamidoethyl)amino)methyl)-4-oxo-4H-pyrido[1,2-a]pyrimidin-8-yl)-2,2′-dichloro-[1,1′-biphenyl]-3-yl)-2-methoxypyridin-3-yl)methyl)amino)ethyl)acetamide;
8-(2,2′-dichloro-3′-(6-methoxy-5-((7-oxo-2,6-diazaspiro[3.4]octan-2-yl)methyl)pyridin-2-yl)-[1,1′-biphenyl]-3-yl)-3-((7-oxo-2,6-diazaspiro[3.4]octan-2-yl)methyl)-4H-pyrido[1,2-a]pyrimidin-4-one;
(S)-8-(3′-(5-(3-aminoazetidin-1-yl)-6-methoxypyrazin-2-yl)-2,2′-dichloro-[1,1′-biphenyl]-3-yl)-3-((((5-oxopyrrolidin-2-yl)methyl)amino)methyl)-4H-pyrido[1,2-a]pyrimidin-4-one;
8-(2′-chloro-3′-(6-ethoxy-5-(((((S)-5-oxopyrrolidin-2-yl)methyl)amino)methyl)pyridin-2-yl)-2-methyl-[1,1′-biphenyl]-3-yl)-3-(((((S)-5-oxopyrrolidin-2-yl)methyl)amino)methyl)-4H-pyrido[1,2-a]pyrimidin-4-one;
8-(2′-chloro-2-methyl-3′-(6-(methylamino)-5-(((((S)-5-oxopyrrolidin-2-yl)methyl)amino)methyl)pyridin-2-yl)-[1,1′-biphenyl]-3-yl)-3-(((((S)-5-oxopyrrolidin-2-yl)methyl)amino)methyl)-4H-pyrido[1,2-a]pyrimidin-4-one;
8-(2′-chloro-3′-(6-(ethylamino)-5-(((((S)-5-oxopyrrolidin-2-yl)methyl)amino)methyl)pyridin-2-yl)-2-methyl-[1,1′-biphenyl]-3-yl)-3-(((((S)-5-oxopyrrolidin-2-yl)methyl)amino)methyl)-4H-pyrido[1,2-a]pyrimidin-4-one;
8-(2,2′-dichloro-3′-(5-(((2-hydroxyethyl)amino)methyl)-6-methoxypyridin-2-yl)-[1,1′-biphenyl]-3-yl)-3-(((2-hydroxyethyl)amino)methyl)-4H-pyrido[1,2-a]pyrimidin-4-one;
8-(2′-chloro-2-methyl-3′-(1-((((S)-5-oxopyrrolidin-2-yl)methyl)amino)-2,3-dihydro-1H-inden-5-yl)-[1,1′-biphenyl]-3-yl)-3-(((((S)-5-oxopyrrolidin-2-yl)methyl)amino)methyl)-4H-pyrido[1,2-a]pyrimidin-4-one;
8-(2′-chloro-3′-(4-methoxy-5-(((((S)-5-oxopyrrolidin-2-yl)methyl)amino)methyl)pyridin-2-yl)-2-methyl-[1,1′-biphenyl]-3-yl)-3-(((((S)-5-oxopyrrolidin-2-yl)methyl)amino)methyl)-4H-pyrido[1,2-a]pyrimidin-4-one;
8-(2′-chloro-2-methyl-3′-(5-(((((S)-5-oxopyrrolidin-2-yl)methyl)amino)methyl)-6-(trifluoromethyl)pyridin-2-yl)-[1,1′-biphenyl]-3-yl)-3-(((((S)-5-oxopyrrolidin-2-yl)methyl)amino)methyl)-4H-pyrido[1,2-a]pyrimidin-4-one;
8-(2′-chloro-2-methyl-3′-(6-methyl-5-(((((S)-5-oxopyrrolidin-2-yl)methyl)amino)methyl)pyridin-2-yl)-[1,1′-biphenyl]-3-yl)-3-(((((S)-5-oxopyrrolidin-2-yl)methyl)amino)methyl)-4H-pyrido[1,2-a]pyrimidin-4-one;
(S)-8-(2,2′-dichloro-3′-(6-methoxy-5-((((5-oxopyrrolidin-2-yl)methyl)amino)methyl)pyridin-2-yl)-[1,1′-biphenyl]-3-yl)-3-(((2-hydroxyethyl)amino)methyl)-4H-pyrido[1,2-a]pyrimidin-4-one;
(S)—N-(2-(((8-(2,2′-dichloro-3′-(6-methoxy-5-((((5-oxopyrrolidin-2-yl)methyl)amino)methyl)pyridin-2-yl)-[1,1′-biphenyl]-3-yl)-4-oxo-4H-pyrido[1,2-a]pyrimidin-3-yl)methyl)amino)ethyl)acetamide;
(S)-5-((((6-(2,2′-dichloro-3′-(2-(((((S)-5-oxopyrrolidin-2-yl)methyl)amino)methyl)pyrazolo[1,5-a]pyrimidin-5-yl)-[1,1′-biphenyl]-3-yl)-2-methoxypyridin-3-yl)methyl)amino)methyl)pyrrolidin-2-one;
(S)-1-((8-(2,2′-dichloro-3′-(5-((3-(hydroxymethyl)azetidin-1-yl)methyl)-6-methoxypyridin-2-yl)-[1,1′-biphenyl]-3-yl)-4-oxo-4H-pyrido[1,2-a]pyrimidin-3-yl)methyl)pyrrolidine-3-carboxylic acid;
1-((8-(2,2′-dichloro-3′-(5-((3-(hydroxymethyl)azetidin-1-yl)methyl)-6-methoxypyridin-2-yl)-[1,1′-biphenyl]-3-yl)-4-oxo-4H-pyrido[1,2-a]pyrimidin-3-yl)methyl)azetidine-3-carboxylic acid;
8-(2,2′-dichloro-3′-(6-methoxy-5-(((((S)-5-oxopyrrolidin-2-yl)methyl)amino)methyl)pyridin-2-yl)-[1,1′-biphenyl]-3-yl)-3-(((((S)-5-oxopyrrolidin-2-yl)methyl)amino)methyl)-4H-quinolizin-4-one;
8-(2,2′-dichloro-5′-fluoro-3′-(6-methoxy-5-(((((S)-5-oxopyrrolidin-2-yl)methyl)amino)methyl)pyridin-2-yl)-[1,1′-biphenyl]-3-yl)-3-(((((S)-5-oxopyrrolidin-2-yl)methyl)amino)methyl)-4H-pyrido[1,2-a]pyrimidin-4-one;
8-(2,2′-dichloro-4″-(((((S)-5-oxopyrrolidin-2-yl)methyl)amino)methyl)-3″-(trifluoromethoxy)-[1,1′:3′,1″-terphenyl]-3-yl)-3-(((((S)-5-oxopyrrolidin-2-yl)methyl)amino)methyl)-4H-pyrido[1,2-a]pyrimidin-4-one;
8-(2′-chloro-2-methyl-4″-(((((S)-5-oxopyrrolidin-2-yl)methyl)amino)methyl)-3″-(trifluoromethoxy)-[1,1′:3′,1″-terphenyl]-3-yl)-3-(((((S)-5-oxopyrrolidin-2-yl)methyl)amino)methyl)-4H-pyrido[1,2-a]pyrimidin-4-one;
8-(2,2′-dichloro-3′-(6-(methylamino)-5-(((((S)-5-oxopyrrolidin-2-yl)methyl)amino)methyl)pyridin-2-yl)-[1,1′-biphenyl]-3-yl)-3-(((((S)-5-oxopyrrolidin-2-yl)methyl)amino)methyl)-4H-pyrido[1,2-a]pyrimidin-4-one;
8-(2,2′-dichloro-3″-methoxy-4″-(((((S)-5-oxopyrrolidin-2-yl)methyl)amino)methyl)-[1,1′:3′,1″-terphenyl]-3-yl)-3-(((((S)-5-oxopyrrolidin-2-yl)methyl)amino)methyl)-4H-pyrido[1,2-a]pyrimidin-4-one;
(5S)-5-((((6-(2,2′-dichloro-3′-(5-(((5-fluoropyridin-3-yl)methyl)amino)-8-methyl-5,6,7,8-tetrahydro-1,8-naphthyridin-2-yl)-[1,1′-biphenyl]-3-yl)-2-methoxypyridin-3-yl)methyl)amino)methyl)pyrrolidin-2-one;
(S)-1-((8-(2,2′-dichloro-3′-(5-((3-(hydroxymethyl)azetidin-1-yl)methyl)-6-methoxypyrazin-2-yl)-[1,1′-biphenyl]-3-yl)-4-oxo-4H-pyrido[1,2-a]pyrimidin-3-yl)methyl)pyrrolidine-3-carboxylic acid;
1-((8-(2,2′-dichloro-3′-(5-((3-(hydroxymethyl)azetidin-1-yl)methyl)-6-methoxypyrazin-2-yl)-[1,1′-biphenyl]-3-yl)-4-oxo-4H-pyrido[1,2-a]pyrimidin-3-yl)methyl)azetidine-3-carboxylic acid;
(S)-1-((8-(2,2′-dichloro-3′-(6-methoxy-5-(((((S)-5-oxopyrrolidin-2-yl)methyl)amino)methyl)pyridin-2-yl)-[1,1′-biphenyl]-3-yl)-4-oxo-4H-pyrido[1,2-a]pyrimidin-3-yl)methyl)pyrrolidine-3-carboxylic acid;
8-(2-chloro-3′-(6-methoxy-5-(((((S)-5-oxopyrrolidin-2-yl)methyl)amino)methyl)pyridin-2-yl)-2′-(trifluoromethyl)-[1,1′-biphenyl]-3-yl)-3-(((((S)-5-oxopyrrolidin-2-yl)methyl)amino)methyl)-4H-pyrido[1,2-a]pyrimidin-4-one;
8-(3′-(6-methoxy-5-(((((S)-5-oxopyrrolidin-2-yl)methyl)amino)methyl)pyridin-2-yl)-2-methyl-2′-(trifluoromethyl)-[1,1′-biphenyl]-3-yl)-3-(((((S)-5-oxopyrrolidin-2-yl)methyl)amino)methyl)-4H-pyrido[1,2-a]pyrimidin-4-one;
8-(2′-chloro-5′-fluoro-3′-(6-methoxy-5-(((((S)-5-oxopyrrolidin-2-yl)methyl)amino)methyl)pyridin-2-yl)-2-methyl-[1,1′-biphenyl]-3-yl)-3-(((((S)-5-oxopyrrolidin-2-yl)methyl)amino)methyl)-4H-pyrido[1,2-a]pyrimidin-4-one;
(S)-1-((8-(2,2′-dichloro-3′-(6-methoxy-5-((((5-oxopyrrolidin-2-yl)methyl)amino)methyl)pyridin-2-yl)-[1,1′-biphenyl]-3-yl)-4-oxo-4H-pyrido[1,2-a]pyrimidin-3-yl)methyl)azetidine-3-carboxylic acid;
(S)-5-((((6-(2,2′-dichloro-3′-(6-(((((S)-5-oxopyrrolidin-2-yl)methyl)amino)methyl)imidazo[1,2-a]pyridin-2-yl)-[1,1′-biphenyl]-3-yl)-2-methoxypyridin-3-yl)methyl)amino)methyl)pyrrolidin-2-one;
2-(((2-(2,2′-dichloro-3′-(5-(((2-hydroxyethyl)amino)methyl)-6-methoxypyridin-2-yl)-[1,1′-biphenyl]-3-yl)imidazo[1,2-a]pyridin-6-yl)methyl)amino)ethan-1-ol;
methyl ((8-(2,2′-dichloro-3′-(6-methoxy-5-(((((S)-5-oxopyrrolidin-2-yl)methyl)amino)methyl)pyridin-2-yl)-[1,1′-biphenyl]-3-yl)-4-oxo-4H-pyrido[1,2-a]pyrimidin-3-yl)methyl)-L-serinate;
(S)-8-(2,2′-dichloro-3′-(5-(((2-hydroxyethyl)amino)methyl)-6-methoxypyridin-2-yl)-[1,1′-biphenyl]-3-yl)-3-((((5-oxopyrrolidin-2-yl)methyl)amino)methyl)-4H-pyrido[1,2-a]pyrimidin-4-one;
(S)—N-(2-(((6-(2,2′-dichloro-3′-(4-oxo-3-((((5-oxopyrrolidin-2-yl)methyl)amino)methyl)-4H-pyrido[1,2-a]pyrimidin-8-yl)-[1,1′-biphenyl]-3-yl)-2-methoxypyridin-3-yl)methyl)amino)ethyl)acetamide;
8-(2,2′-dichloro-3″-fluoro-5″-methoxy-4″-(((((S)-5-oxopyrrolidin-2-yl)methyl)amino)methyl)-[1,1′:3′,1″-terphenyl]-3-yl)-3-(((((S)-5-oxopyrrolidin-2-yl)methyl)amino)methyl)-4H-pyrido[1,2-a]pyrimidin-4-one;
8-(2,2′-dichloro-2″-fluoro-3″-methoxy-4″-(((((S)-5-oxopyrrolidin-2-yl)methyl)amino)methyl)-[1,1′:3′,1″-terphenyl]-3-yl)-3-(((((S)-5-oxopyrrolidin-2-yl)methyl)amino)methyl)-4H-pyrido[1,2-a]pyrimidin-4-one;
8-(2,2′-dichloro-2″-fluoro-5″-methoxy-4″-(((((S)-5-oxopyrrolidin-2-yl)methyl)amino)methyl)-[1,1′:3′,1″-terphenyl]-3-yl)-3-(((((S)-5-oxopyrrolidin-2-yl)methyl)amino)methyl)-4H-pyrido[1,2-a]pyrimidin-4-one;
8-(2,2′-dichloro-3′-(6-methoxy-5-(((((S)-5-oxopyrrolidin-2-yl)methyl)amino)methyl)pyrazin-2-yl)-[1,1′-biphenyl]-3-yl)-3-(((((S)-5-oxopyrrolidin-2-yl)methyl)amino)methyl)-4H-quinolizin-4-one;
8-(2,2′-dichloro-3′-(6-(methylthio)-5-(((((S)-5-oxopyrrolidin-2-yl)methyl)amino)methyl)pyridin-2-yl)-[1,1′-biphenyl]-3-yl)-3-(((((S)-5-oxopyrrolidin-2-yl)methyl)amino)methyl)-4H-pyrido[1,2-a]pyrimidin-4-one;
8-(2,2′-dichloro-3′-(6-cyclopropoxy-5-(((((S)-5-oxopyrrolidin-2-yl)methyl)amino)methyl)pyridin-2-yl)-[1,1′-biphenyl]-3-yl)-3-(((((S)-5-oxopyrrolidin-2-yl)methyl)amino)methyl)-4H-pyrido[1,2-a]pyrimidin-4-one;
8-(2,2′-dichloro-3′-(difluoromethoxy)-4″-(((((S)-5-oxopyrrolidin-2-yl)methyl)amino)methyl)-[1,1′:3′,1″-terphenyl]-3-yl)-3-(((((S)-5-oxopyrrolidin-2-yl)methyl)amino)methyl)-4H-pyrido[1,2-a]pyrimidin-4-one;
(S)-8-(2,2′-dichloro-3′-(6-methoxy-5-((((5-oxopyrrolidin-2-yl)methyl)amino)methyl)pyridin-2-yl)-[1,1′-biphenyl]-3-yl)-3-((3-(2-hydroxypropan-2-yl)azetidin-1-yl)methyl)-4H-pyrido[1,2-a]pyrimidin-4-one;
(S)-8-(2,2′-dichloro-3′-(5-((3-(2-hydroxypropan-2-yl)azetidin-1-yl)methyl)-6-methoxypyridin-2-yl)-[1,1′-biphenyl]-3-yl)-3-((((5-oxopyrrolidin-2-yl)methyl)amino)methyl)-4H-pyrido[1,2-a]pyrimidin-4-one;
methyl ((6-(2,2′-dichloro-3′-(4-oxo-3-(((((S)-5-oxopyrrolidin-2-yl)methyl)amino)methyl)-4H-pyrido[1,2-a]pyrimidin-8-yl)-[1,1′-biphenyl]-3-yl)-2-methoxypyridin-3-yl)methyl)-L-serinate;
((6-(2,2′-dichloro-3′-(4-oxo-3-(((((S)-5-oxopyrrolidin-2-yl)methyl)amino)methyl)-4H-pyrido[1,2-a]pyrimidin-8-yl)-[1,1′-biphenyl]-3-yl)-2-methoxypyridin-3-yl)methyl)-L-serine;
(S)-1-(((6-(2,2′-dichloro-3′-(4-oxo-3-((((5-oxopyrrolidin-2-yl)methyl)amino)methyl)-4H-pyrido[1,2-a]pyrimidin-8-yl)-[1,1′-biphenyl]-3-yl)-2-methoxypyridin-3-yl)methyl)amino)cyclopropane-1-carboxylic acid;
(S)-1-((8-(2,2′-dichloro-3′-(6-methoxy-5-(((((S)-5-oxopyrrolidin-2-yl)methyl)amino)methyl)pyrazin-2-yl)-[1,1′-biphenyl]-3-yl)-4-oxo-4H-pyrido[1,2-a]pyrimidin-3-yl)methyl)pyrrolidine-3-carboxylic acid;
(S)-1-((8-(2,2′-dichloro-3′-(6-methoxy-5-((((5-oxopyrrolidin-2-yl)methyl)amino)methyl)pyrazin-2-yl)-[1,1′-biphenyl]-3-yl)-4-oxo-4H-pyrido[1,2-a]pyrimidin-3-yl)methyl)azetidine-3-carboxylic acid;
(S)-1-((6-(2,2′-dichloro-3′-(4-oxo-3-(((((S)-5-oxopyrrolidin-2-yl)methyl)amino)methyl)-4H-pyrido[1,2-a]pyrimidin-8-yl)-[1,1′-biphenyl]-3-yl)-2-methoxypyridin-3-yl)methyl)pyrrolidine-3-carboxylic acid;
(S)-1-((6-(2,2′-dichloro-3′-(4-oxo-3-((((5-oxopyrrolidin-2-yl)methyl)amino)methyl)-4H-pyrido[1,2-a]pyrimidin-8-yl)-[1,1′-biphenyl]-3-yl)-2-methoxypyridin-3-yl)methyl)azetidine-3-carboxylic acid;
(S)-1-(((8-(2,2′-dichloro-3′-(6-methoxy-5-((((5-oxopyrrolidin-2-yl)methyl)amino)methyl)pyridin-2-yl)-[1,1′-biphenyl]-3-yl)-4-oxo-4H-pyrido[1,2-a]pyrimidin-3-yl)methyl)amino)cyclopropane-1-carboxylic acid;
methyl (S)-2-((6-(2,2′-dichloro-3′-(4-oxo-3-((((5-oxopyrrolidin-2-yl)methyl)amino)methyl)-4H-pyrido[1,2-a]pyrimidin-8-yl)-[1,1′-biphenyl]-3-yl)-2-methoxypyridin-3-yl)methyl)-2-azabicyclo[2.2.2]octane-4-carboxylate;
(S)-2-((6-(2,2′-dichloro-3′-(4-oxo-3-((((5-oxopyrrolidin-2-yl)methyl)amino)methyl)-4H-pyrido[1,2-a]pyrimidin-8-yl)-[1,1′-biphenyl]-3-yl)-2-methoxypyridin-3-yl)methyl)-2-azabicyclo[2.2.2]octane-4-carboxylic acid;
(S)-5-((((3-chloro-6-(2,2′-dichloro-3′-(6-methoxy-5-(((((S)-5-oxopyrrolidin-2-yl)methyl)amino)methyl)pyridin-2-yl)-[1,1′-biphenyl]-3-yl)thieno[3,2-b]pyridin-2-yl)methyl)amino)methyl)pyrrolidin-2-one;
methyl (S)-((6-(2,2′-dichloro-3′-(4-oxo-3-((((5-oxopyrrolidin-2-yl)methyl)amino)methyl)-4H-pyrido[1,2-a]pyrimidin-8-yl)-[1,1′-biphenyl]-3-yl)-2-methoxypyridin-3-yl)methyl)glycinate;
(S)-((6-(2,2′-dichloro-3′-(4-oxo-3-((((5-oxopyrrolidin-2-yl)methyl)amino)methyl)-4H-pyrido[1,2-a]pyrimidin-8-yl)-[1,1′-biphenyl]-3-yl)-2-methoxypyridin-3-yl)methyl)glycine;
(S)-4-(((8-(2,2′-dichloro-3′-(6-methoxy-5-(((((S)-5-oxopyrrolidin-2-yl)methyl)amino)methyl)pyridin-2-yl)-[1,1′-biphenyl]-3-yl)-4-oxo-4H-pyrido[1,2-a]pyrimidin-3-yl)methyl)amino)-3-hydroxybutanoic acid;
(S)-2-((8-(2,2′-dichloro-3′-(6-methoxy-5-((((5-oxopyrrolidin-2-yl)methyl)amino)methyl)pyridin-2-yl)-[1,1′-biphenyl]-3-yl)-4-oxo-4H-pyrido[1,2-a]pyrimidin-3-yl)methyl)-2-azabicyclo[2.2.2]octane-4-carboxylic acid;
((8-(2,2′-dichloro-3′-(6-methoxy-5-(((((S)-5-oxopyrrolidin-2-yl)methyl)amino)methyl)pyridin-2-yl)-[1,1′-biphenyl]-3-yl)-4-oxo-4H-pyrido[1,2-a]pyrimidin-3-yl)methyl)-L-proline;
(S)-1-((8-(2,2′-dichloro-3′-(6-methoxy-5-(((((S)-5-oxopyrrolidin-2-yl)methyl)amino)methyl)pyridin-2-yl)-[1,1′-biphenyl]-3-yl)-4-oxo-4H-pyrido[1,2-a]pyrimidin-3-yl)methyl)piperidine-2-carboxylic acid;
(S)-1-((8-(2,2′-dichloro-3′-(6-methoxy-5-(((((S)-5-oxopyrrolidin-2-yl)methyl)amino)methyl)pyridin-2-yl)-[1,1′-biphenyl]-3-yl)-4-oxo-4H-pyrido[1,2-a]pyrimidin-3-yl)methyl)piperidine-3-carboxylic acid;
8-(2,2′-dichloro-3′-(6-methoxy-5-(((((S)-5-oxopyrrolidin-2-yl)methyl)amino)methyl)pyridin-2-yl)-[1,1′-biphenyl]-3-yl)-3-((((S)-2-hydroxypropyl)amino)methyl)-4H-pyrido[1,2-a]pyrimidin-4-one;
8-(2,2′-dichloro-3′-(6-methoxy-5-(((((S)-5-oxopyrrolidin-2-yl)methyl)amino)methyl)pyridin-2-yl)-[1,1′-biphenyl]-3-yl)-3-((((R)-2-hydroxypropyl)amino)methyl)-4H-pyrido[1,2-a]pyrimidin-4-one;
(S)-8-(2,2′-dichloro-3′-(6-methoxy-5-((((5-oxopyrrolidin-2-yl)methyl)amino)methyl)pyridin-2-yl)-[1,1′-biphenyl]-3-yl)-3-(((2-hydroxy-2-methylpropyl)amino)methyl)-4H-pyrido[1,2-a]pyrimidin-4-one;
(2R,4R)-1-((8-(2,2′-dichloro-3′-(6-methoxy-5-(((((S)-5-oxopyrrolidin-2-yl)methyl)amino)methyl)pyridin-2-yl)-[1,1′-biphenyl]-3-yl)-4-oxo-4H-pyrido[1,2-a]pyrimidin-3-yl)methyl)-4-hydroxypyrrolidine-2-carboxylic acid;
(S)-1-((6-(2,2′-dichloro-3′-(3-((3-(hydroxymethyl)azetidin-1-yl)methyl)-4-oxo-4H-pyrido[1,2-a]pyrimidin-8-yl)-[1,1′-biphenyl]-3-yl)-2-methoxypyridin-3-yl)methyl)pyrrolidine-3-carboxylic acid;
1-((6-(2,2′-dichloro-3′-(3-((3-(hydroxymethyl)azetidin-1-yl)methyl)-4-oxo-4H-pyrido[1,2-a]pyrimidin-8-yl)-[1,1′-biphenyl]-3-yl)-2-methoxypyridin-3-yl)methyl)azetidine-3-carboxylic acid;
methyl (S)-4-(((8-(2,2′-dichloro-3′-(6-methoxy-5-((((5-oxopyrrolidin-2-yl)methyl)amino)methyl)pyridin-2-yl)-[1,1′-biphenyl]-3-yl)-4-oxo-4H-pyrido[1,2-a]pyrimidin-3-yl)methyl)amino)butanoate;
(S)-8-(2,2′-dichloro-3′-(6-methoxy-5-((((5-oxopyrrolidin-2-yl)methyl)amino)methyl)pyridin-2-yl)-[1,1′-biphenyl]-3-yl)-3-(((2,4-dimethoxybenzyl)amino)methyl)-4H-pyrido[1,2-a]pyrimidin-4-one;
(S)-2-(1-(((8-(2,2′-dichloro-3′-(6-methoxy-5-((((5-oxopyrrolidin-2-yl)methyl)amino)methyl)pyridin-2-yl)-[1,1′-biphenyl]-3-yl)-4-oxo-4H-pyrido[1,2-a]pyrimidin-3-yl)methyl)amino)cyclopropyl)acetic acid;
8-(2,2′-dichloro-5-fluoro-3′-(6-methoxy-5-(((((S)-5-oxopyrrolidin-2-yl)methyl)amino)methyl)pyridin-2-yl)-[1,1′-biphenyl]-3-yl)-3-(((((S)-5-oxopyrrolidin-2-yl)methyl)amino)methyl)-4H-pyrido[1,2-a]pyrimidin-4-one;
3-(((((S)-5-oxopyrrolidin-2-yl)methyl)amino)methyl)-8-(2,2′,5-trichloro-3′-(6-methoxy-5-(((((S)-5-oxopyrrolidin-2-yl)methyl)amino)methyl)pyridin-2-yl)-[1,1′-biphenyl]-3-yl)-4H-pyrido[1,2-a]pyrimidin-4-one;
8-(2,2′-dichloro-3′-(5-((((1r,3r)-3-hydroxy-3-methylcyclobutyl)amino)methyl)-6-methoxypyridin-2-yl)-[1,1′-biphenyl]-3-yl)-3-(((((S)-5-oxopyrrolidin-2-yl)methyl)amino)methyl)-4H-pyrido[1,2-a]pyrimidin-4-one;
8-(2,2′-dichloro-3′-(6-methoxy-5-(((((S)-5-oxopyrrolidin-2-yl)methyl)amino)methyl)pyridin-2-yl)-[1,1′-biphenyl]-3-yl)-3-((((1r,3r)-3-hydroxy-3-methylcyclobutyl)amino)methyl)-4H-pyrido[1,2-a]pyrimidin-4-one;
8-(2,2′-dichloro-3′-(6-methoxy-5-(((((S)-5-oxopyrrolidin-2-yl)methyl)amino)methyl)pyridin-2-yl)-[1,1′-biphenyl]-3-yl)-3-(((S)-3-hydroxypyrrolidin-1-yl)methyl)-4H-pyrido[1,2-a]pyrimidin-4-one;
2′-chloro-3-(6-methoxy-5-(((((S)-5-oxopyrrolidin-2-yl)methyl)amino)methyl)pyridin-2-yl)-3′-(4-oxo-3-(((((S)-5-oxopyrrolidin-2-yl)methyl)amino)methyl)-4H-pyrido[1,2-a]pyrimidin-8-yl)-[1,1′-biphenyl]-2-carbonitrile;
8-(2,2′-dichloro-5′-methoxy-3′-(6-methoxy-5-(((((S)-5-oxopyrrolidin-2-yl)methyl)amino)methyl)pyridin-2-yl)-[1,1′-biphenyl]-3-yl)-3-(((((S)-5-oxopyrrolidin-2-yl)methyl)amino)methyl)-4H-pyrido[1,2-a]pyrimidin-4-one;
8-(2,2′-dichloro-3′-(4-fluoro-6-methoxy-5-(((((S)-5-oxopyrrolidin-2-yl)methyl)amino)methyl)pyridin-2-yl)-[1,1′-biphenyl]-3-yl)-3-(((((S)-5-oxopyrrolidin-2-yl)methyl)amino)methyl)-4H-pyrido[1,2-a]pyrimidin-4-one;
(2S,4S)-1-((8-(2,2′-dichloro-3′-(6-methoxy-5-(((((S)-5-oxopyrrolidin-2-yl)methyl)amino)methyl)pyridin-2-yl)-[1,1′-biphenyl]-3-yl)-4-oxo-4H-pyrido[1,2-a]pyrimidin-3-yl)methyl)-4-hydroxypyrrolidine-2-carboxylic acid;
(2R,4S)-1-((8-(2,2′-dichloro-3′-(6-methoxy-5-(((((S)-5-oxopyrrolidin-2-yl)methyl)amino)methyl)pyridin-2-yl)-[1,1′-biphenyl]-3-yl)-4-oxo-4H-pyrido[1,2-a]pyrimidin-3-yl)methyl)-4-hydroxypyrrolidine-2-carboxylic acid;
N-((8-(2,2′-dichloro-3′-(6-methoxy-5-(((((S)-5-oxopyrrolidin-2-yl)methyl)amino)methyl)pyridin-2-yl)-[1,1′-biphenyl]-3-yl)-4-oxo-4H-pyrido[1,2-a]pyrimidin-3-yl)methyl)-O-methyl-D-serine;
((8-(2,2′-dichloro-3′-(6-methoxy-5-(((((S)-5-oxopyrrolidin-2-yl)methyl)amino)methyl)pyridin-2-yl)-[1,1′-biphenyl]-3-yl)-4-oxo-4H-pyrido[1,2-a]pyrimidin-3-yl)methyl)-L-serine;
8-(2,2′-dichloro-3′-(6-methoxy-5-(((((S)-5-oxopyrrolidin-2-yl)methyl)amino)methyl)pyridin-2-yl)-[1,1′-biphenyl]-3-yl)-3-((6-oxohexahydropyrrolo[1,2-a]pyrazin-2(1H)-yl)methyl)-4H-pyrido[1,2-a]pyrimidin-4-one;
8-(2,2′-dichloro-3′-(6-methoxy-5-(((((S)-5-oxopyrrolidin-2-yl)methyl)amino)methyl)pyridin-2-yl)-[1,1′-biphenyl]-3-yl)-3-((2-(hydroxymethyl)pyrrolidin-1-yl)methyl)-4H-pyrido[1,2-a]pyrimidin-4-one;
(S)-3-(((8-(2,2′-dichloro-3′-(6-methoxy-5-((((5-oxopyrrolidin-2-yl)methyl)amino)methyl)pyridin-2-yl)-[1,1′-biphenyl]-3-yl)-4-oxo-4H-pyrido[1,2-a]pyrimidin-3-yl)methyl)amino)-3-methylbutanoic acid;
5-((6-(2,2′-dichloro-3′-(6-methoxy-5-(((((S)-5-oxopyrrolidin-2-yl)methyl)amino)methyl)pyridin-2-yl)-[1,1′-biphenyl]-3-yl)-8-methoxy-3,4-dihydroisoquinolin-2(1H)-yl)methyl)pyrrolidin-2-one;
8-(2,2′-dichloro-3′-(6-methoxy-5-(((((S)-5-oxopyrrolidin-2-yl)methyl)amino)methyl)pyridin-2-yl)-[1,1′-biphenyl]-3-yl)-2-(((((S)-5-oxopyrrolidin-2-yl)methyl)amino)methyl)-4H-pyrido[1,2-a]pyrimidin-4-one;
(S)-8-(2,2′-dichloro-3′-(5-((3-hydroxy-3-methylazetidin-1-yl)methyl)-6-methoxypyridin-2-yl)-[1,1′-biphenyl]-3-yl)-3-((((5-oxopyrrolidin-2-yl)methyl)amino)methyl)-4H-pyrido[1,2-a]pyrimidin-4-one;
8-(2,2′-dichloro-3′-(5-((((1r,3r)-3-hydroxycyclobutyl)amino)methyl)-6-methoxypyridin-2-yl)-[1,1′-biphenyl]-3-yl)-3-(((((S)-5-oxopyrrolidin-2-yl)methyl)amino)methyl)-4H-pyrido[1,2-a]pyrimidin-4-one;
(S)-8-(3′-(5-((3,3-bis(hydroxymethyl)azetidin-1-yl)methyl)-6-methoxypyridin-2-yl)-2,2′-dichloro-[1,1′-biphenyl]-3-yl)-3-((((5-oxopyrrolidin-2-yl)methyl)amino)methyl)-4H-pyrido[1,2-a]pyrimidin-4-one;
(S)-4-(((8-(2,2′-dichloro-3′-(6-methoxy-5-((((5-oxopyrrolidin-2-yl)methyl)amino)methyl)pyridin-2-yl)-[1,1′-biphenyl]-3-yl)-4-oxo-4H-pyrido[1,2-a]pyrimidin-3-yl)methyl)amino)butanoic acid;
8-(2,2′-dichloro-3′-(6-methoxy-5-(((((S)-5-oxopyrrolidin-2-yl)methyl)amino)methyl)pyridin-2-yl)-[1,1′-biphenyl]-3-yl)-3-(5-hydroxy-1,4,5,6-tetrahydropyrimidin-2-yl)-4H-pyrido[1,2-a]pyrimidin-4-one;
methyl (S)-1-((8-(2,2′-dichloro-3′-(6-methoxy-5-((((5-oxopyrrolidin-2-yl)methyl)amino)methyl)pyridin-2-yl)-[1,1′-biphenyl]-3-yl)-4-oxo-4H-pyrido[1,2-a]pyrimidin-3-yl)methyl)azetidine-3-carboxylate;
8-(2,2′-dichloro-3′-(6-methoxy-5-(((((S)-5-oxopyrrolidin-2-yl)methyl)amino)methyl)pyridin-2-yl)-[1,1′-biphenyl]-3-yl)-3-((((3R,4S)-3-hydroxytetrahydro-2H-pyran-4-yl)amino)methyl)-4H-pyrido[1,2-a]pyrimidin-4-one;
8-(2,2′-dichloro-3′-(6-methoxy-5-(((((S)-5-oxopyrrolidin-2-yl)methyl)amino)methyl)pyridin-2-yl)-[1,1′-biphenyl]-3-yl)-2-methyl-3-(((((S)-5-oxopyrrolidin-2-yl)methyl)amino)methyl)-4H-pyrido[1,2-a]pyrimidin-4-one;
8-(2,2′-dichloro-3′-(5-(((S)-3-hydroxypyrrolidin-1-yl)methyl)-6-methoxypyridin-2-yl)-[1,1′-biphenyl]-3-yl)-3-(((S)-3-hydroxypyrrolidin-1-yl)methyl)-4H-pyrido[1,2-a]pyrimidin-4-one;
(2S,4R)-1-((8-(2,2′-dichloro-3′-(6-methoxy-5-(((((S)-5-oxopyrrolidin-2-yl)methyl)amino)methyl)pyridin-2-yl)-[1,1′-biphenyl]-3-yl)-4-oxo-4H-pyrido[1,2-a]pyrimidin-3-yl)methyl)-4-hydroxypyrrolidine-2-carboxylic acid;
methyl (S)-2-(8-(2,2′-dichloro-3′-(6-methoxy-5-((((5-oxopyrrolidin-2-yl)methyl)amino)methyl)pyridin-2-yl)-[1,1′-biphenyl]-3-yl)-4-oxo-4H-pyrido[1,2-a]pyrimidin-3-yl)-1H-imidazole-5-carboxylate;
(S)-1-((8-(2,2′-dichloro-3′-(5-(((2-hydroxyethyl)amino)methyl)-6-methoxypyridin-2-yl)-[1,1′-biphenyl]-3-yl)-4-oxo-4H-pyrido[1,2-a]pyrimidin-3-yl)methyl)-2-methylpyrrolidine-2-carboxylic acid;
8-(2,2′-dichloro-3′-(6-methoxy-5-(((((S)-5-oxopyrrolidin-2-yl)methyl)amino)methyl)pyridin-2-yl)-[1,1′-biphenyl]-3-yl)-3-(((R)-3-hydroxypyrrolidin-1-yl)methyl)-4H-pyrido[1,2-a]pyrimidin-4-one;
8-(2,2′-dichloro-3′-(5-(((R)-3-hydroxypyrrolidin-1-yl)methyl)-6-methoxypyridin-2-yl)-[1,1′-biphenyl]-3-yl)-3-(((((S)-5-oxopyrrolidin-2-yl)methyl)amino)methyl)-4H-pyrido[1,2-a]pyrimidin-4-one;
methyl 2-(8-(2,2′-dichloro-3′-(6-methoxy-5-(((((S)-5-oxopyrrolidin-2-yl)methyl)amino)methyl)pyridin-2-yl)-[1,1′-biphenyl]-3-yl)-4-oxo-4H-pyrido[1,2-a]pyrimidin-3-yl)-4,5-dihydro-1H-imidazole-5-carboxylate;
(S)-1-((8-(2,2′-dichloro-3′-(5-(((2-hydroxyethyl)amino)methyl)-6-methoxypyridin-2-yl)-[1,1′-biphenyl]-3-yl)-4-oxo-4H-pyrido[1,2-a]pyrimidin-3-yl)methyl)pyrrolidine-3-carboxylic acid;
(S)-1-(5-chloro-2-(2-cyanoethoxy)-4-((E)-2-(2,2′-dichloro-3′-(4-oxo-3-(((((S)-5-oxopyrrolidin-2-yl)methyl)amino)methyl)-4H-pyrido[1,2-a]pyrimidin-8-yl)-[1,1′-biphenyl]-3-yl)vinyl)benzyl)pyrrolidine-3-carboxylic acid;
8-(2,2′-dichloro-3′-(5-((((3R,4S)-3-hydroxytetrahydro-2H-pyran-4-yl)amino)methyl)-6-methoxypyridin-2-yl)-[1,1′-biphenyl]-3-yl)-3-(((((S)-5-oxopyrrolidin-2-yl)methyl)amino)methyl)-4H-pyrido[1,2-a]pyrimidin-4-one;
8-(2,2′-dichloro-3′-(5-(((S)-3-hydroxypyrrolidin-1-yl)methyl)-6-methoxypyridin-2-yl)-[1,1′-biphenyl]-3-yl)-3-(((((S)-5-oxopyrrolidin-2-yl)methyl)amino)methyl)-4H-pyrido[1,2-a]pyrimidin-4-one;
8-(2,2′-dichloro-3′-(5-methoxy-6-(((((S)-5-oxopyrrolidin-2-yl)methyl)amino)methyl)quinolin-2-yl)-[1,1′-biphenyl]-3-yl)-3-(((((S)-5-oxopyrrolidin-2-yl)methyl)amino)methyl)-4H-pyrido[1,2-a]pyrimidin-4-one;
8-(2-chloro-3-((E)-1-((3-chloro-6-methoxy-5-(((((S)-5-oxopyrrolidin-2-yl)methyl)amino)methyl)pyridin-2-yl)methylene)-2,3-dihydro-1H-inden-4-yl)phenyl)-3-(((((S)-5-oxopyrrolidin-2-yl)methyl)amino)methyl)-4H-pyrido[1,2-a]pyrimidin-4-one;
8-(2,2′-dichloro-3′-(5-(5-hydroxy-1,4,5,6-tetrahydropyrimidin-2-yl)-6-methoxypyridin-2-yl)-[1,1′-biphenyl]-3-yl)-3-(((((S)-5-oxopyrrolidin-2-yl)methyl)amino)methyl)-4H-pyrido[1,2-a]pyrimidin-4-one;
2-(8-(2,2′-dichloro-3′-(6-methoxy-5-(((((S)-5-oxopyrrolidin-2-yl)methyl)amino)methyl)pyridin-2-yl)-[1,1′-biphenyl]-3-yl)-4-oxo-4H-pyrido[1,2-a]pyrimidin-3-yl)-4,5-dihydro-1H-imidazole-5-carboxylic acid;
2-(6-(2,2′-dichloro-3′-(4-oxo-3-(((((S)-5-oxopyrrolidin-2-yl)methyl)amino)methyl)-4H-pyrido[1,2-a]pyrimidin-8-yl)-[1,1′-biphenyl]-3-yl)-2-methoxypyridin-3-yl)-3,4,5,6-tetrahydropyrimidine-4-carboxylic acid;
8-(2,2′-dichloro-3′-(5-((((S)-2-hydroxypropyl)amino)methyl)-6-methoxypyridin-2-yl)-[1,1′-biphenyl]-3-yl)-3-((((S)-2-hydroxypropyl)amino)methyl)-2-methyl-4H-pyrido[1,2-a]pyrimidin-4-one;
(S)-3-(((8-(2,2′-dichloro-3′-(6-methoxy-5-((((5-oxopyrrolidin-2-yl)methyl)amino)methyl)pyridin-2-yl)-[1,1′-biphenyl]-3-yl)-4-oxo-4H-pyrido[1,2-a]pyrimidin-3-yl)methyl)amino)propanoic acid;
8-(2,2′-dichloro-3′-(6-methoxy-5-(((((S)-5-oxopyrrolidin-2-yl)methyl)amino)methyl)pyridin-2-yl)-[1,1′-biphenyl]-3-yl)-6-methyl-3-(((((S)-5-oxopyrrolidin-2-yl)methyl)amino)methyl)-4H-pyrido[1,2-a]pyrimidin-4-one;
(S)-2-(((8-(2,2′-dichloro-3′-(6-methoxy-5-((((5-oxopyrrolidin-2-yl)methyl)amino)methyl)pyridin-2-yl)-[1,1′-biphenyl]-3-yl)-4-oxo-4H-pyrido[1,2-a]pyrimidin-3-yl)methyl)amino)-2-methylpropanoic acid;
8-(2,2′-dichloro-3′-(5-((((S)-2-hydroxypropyl)amino)methyl)-6-methoxypyridin-2-yl)-[1,1′-biphenyl]-3-yl)-3-((((S)-2-hydroxypropyl)amino)methyl)-4H-pyrido[1,2-a]pyrimidin-4-one;
8-(2,2′-dichloro-3′-(5-((((R)-2-hydroxypropyl)amino)methyl)-6-methoxypyridin-2-yl)-[1,1′-biphenyl]-3-yl)-3-((((R)-2-hydroxypropyl)amino)methyl)-4H-pyrido[1,2-a]pyrimidin-4-one;
(S)—N-((8-(2,2′-dichloro-3′-(6-methoxy-5-((((5-oxopyrrolidin-2-yl)methyl)amino)methyl)pyridin-2-yl)-[1,1′-biphenyl]-3-yl)-4-oxo-4H-pyrido[1,2-a]pyrimidin-3-yl)methyl)-N-methylglycine;
1-((8-(2,2′-dichloro-3′-(5-(((2-hydroxyethyl)amino)methyl)-6-methoxypyridin-2-yl)-[1,1′-biphenyl]-3-yl)-4-oxo-4H-pyrido[1,2-a]pyrimidin-3-yl)methyl)azetidine-3-carboxylic acid;
(S)-((8-(2,2′-dichloro-3′-(6-methoxy-5-((((5-oxopyrrolidin-2-yl)methyl)amino)methyl)pyridin-2-yl)-[1,1′-biphenyl]-3-yl)-4-oxo-4H-pyrido[1,2-a]pyrimidin-3-yl)methyl)glycine;
N-((8-(2,2′-dichloro-3′-(6-methoxy-5-(((((S)-5-oxopyrrolidin-2-yl)methyl)amino)methyl)pyridin-2-yl)-[1,1′-biphenyl]-3-yl)-4-oxo-4H-pyrido[1,2-a]pyrimidin-3-yl)methyl)-N-methyl-L-alanine;
((8-(2,2′-dichloro-3′-(5-(((2-hydroxyethyl)amino)methyl)-6-methoxypyridin-2-yl)-[1,1′-biphenyl]-3-yl)-4-oxo-4H-pyrido[1,2-a]pyrimidin-3-yl)methyl)-L-serine;
methyl (S)-1-((8-(2,2′-dichloro-3′-(6-methoxy-5-(((((S)-5-oxopyrrolidin-2-yl)methyl)amino)methyl)pyridin-2-yl)-[1,1′-biphenyl]-3-yl)-4-oxo-4H-pyrido[1,2-a]pyrimidin-3-yl)methyl)pyrrolidine-3-carboxylate;
8-(2,2′-dichloro-3′-(6-methoxy-5-(((((S)-5-oxopyrrolidin-2-yl)methyl)amino)methyl)pyridin-2-yl)-[1,1′-biphenyl]-3-yl)-3-((((S)-2-hydroxypropyl)amino)methyl)-2-methyl-4H-pyrido[1,2-a]pyrimidin-4-one;
(S)-5-((((8-chloro-2-(2,2′-dichloro-3′-(6-methoxy-5-(((((S)-5-oxopyrrolidin-2-yl)methyl)amino)methyl)pyridin-2-yl)-[1,1′-biphenyl]-3-yl)-5-methoxyquinolin-6-yl)methyl)amino)methyl)pyrrolidin-2-one;
8-(2,2′-dichloro-3′-(5-((((S)-2-hydroxypropyl)amino)methyl)-6-methoxypyridin-2-yl)-[1,1′-biphenyl]-3-yl)-3-((((R)-2-hydroxypropyl)amino)methyl)-4H-pyrido[1,2-a]pyrimidin-4-one;
8-(2,2′-dichloro-3′-(5-((((R)-2-hydroxypropyl)amino)methyl)-6-methoxypyridin-2-yl)-[1,1′-biphenyl]-3-yl)-3-((((S)-2-hydroxypropyl)amino)methyl)-4H-pyrido[1,2-a]pyrimidin-4-one;
8-(2,2′-dichloro-3′-(5-((((S)-2-hydroxypropyl)amino)methyl)-6-methoxypyridin-2-yl)-[1,1′-biphenyl]-3-yl)-3-(((((S)-5-oxopyrrolidin-2-yl)methyl)amino)methyl)-4H-pyrido[1,2-a]pyrimidin-4-one;
8-(2,2′-dichloro-3′-(5-((((R)-2-hydroxypropyl)amino)methyl)-6-methoxypyridin-2-yl)-[1,1′-biphenyl]-3-yl)-3-(((((S)-5-oxopyrrolidin-2-yl)methyl)amino)methyl)-4H-pyrido[1,2-a]pyrimidin-4-one;
2-(8-(2,2′-dichloro-3′-(6-methoxy-5-(((((S)-5-oxopyrrolidin-2-yl)methyl)amino)methyl)pyridin-2-yl)-[1,1′-biphenyl]-3-yl)-4-oxo-4H-pyrido[1,2-a]pyrimidin-3-yl)-3,4,5,6-tetrahydropyrimidine-4-carboxylic acid;
(S)-2-(((8-(2,2′-dichloro-3′-(6-methoxy-5-((((5-oxopyrrolidin-2-yl)methyl)amino)methyl)pyridin-2-yl)-[1,1′-biphenyl]-3-yl)-4-oxo-4H-pyrido[1,2-a]pyrimidin-3-yl)methyl)(methyl)amino)-N-methylacetamide;
8-(2,2′-dichloro-3′-(6-methoxy-5-(((((S)-5-oxopyrrolidin-2-yl)methyl)amino)methyl)pyridin-2-yl)-[1,1′-biphenyl]-3-yl)-2-methoxy-3-(((((S)-5-oxopyrrolidin-2-yl)methyl)amino)methyl)-4H-pyrido[1,2-a]pyrimidin-4-one;
(S)-2-(((8-(2,2′-dichloro-3′-(6-methoxy-5-((((5-oxopyrrolidin-2-yl)methyl)amino)methyl)pyridin-2-yl)-[1,1′-biphenyl]-3-yl)-4-oxo-4H-pyrido[1,2-a]pyrimidin-3-yl)methyl)amino)-N-methylacetamide;
8-(2,2′-dichloro-3′-(8-chloro-5-methoxy-6-(((((S)-5-oxopyrrolidin-2-yl)methyl)amino)methyl)quinolin-2-yl)-[1,1′-biphenyl]-3-yl)-3-(((((S)-5-oxopyrrolidin-2-yl)methyl)amino)methyl)-4H-pyrido[1,2-a]pyrimidin-4-one;
8-(2,2′-dichloro-3′-(6-methoxy-5-(((((S)-5-oxopyrrolidin-2-yl)methyl)amino)methyl)pyridin-2-yl)-[1,1′-biphenyl]-3-yl)-3-((((1r,3r)-3-hydroxy-3-methylcyclobutyl)amino)methyl)-2-methyl-4H-pyrido[1,2-a]pyrimidin-4-one;
8-(2,2′-dichloro-3′-(5-((((1r,3r)-3-hydroxy-3-methylcyclobutyl)amino)methyl)-6-methoxypyridin-2-yl)-[1,1′-biphenyl]-3-yl)-3-((((1r,3r)-3-hydroxy-3-methylcyclobutyl)amino)methyl)-2-methyl-4H-pyrido[1,2-a]pyrimidin-4-one;
8-(2,2′-dichloro-3′-(6-methoxy-5-(((((S)-5-oxopyrrolidin-2-yl)methyl)amino)methyl)pyridin-2-yl)-[1,1′-biphenyl]-3-yl)-3-((((6-oxopiperidin-2-yl)methyl)amino)methyl)-4H-pyrido[1,2-a]pyrimidin-4-one;
(S)-8-(2,2′-dichloro-3′-(6-methoxy-5-((((5-oxopyrrolidin-2-yl)methyl)amino)methyl)pyridin-2-yl)-[1,1′-biphenyl]-3-yl)-3-(((2-(2-oxoimidazolidin-1-yl)ethyl)amino)methyl)-4H-pyrido[1,2-a]pyrimidin-4-one;
8-(2′-chloro-3′-(6-methoxy-5-(((((S)-5-oxopyrrolidin-2-yl)methyl)amino)methyl)pyridin-2-yl)-2-(trifluoromethyl)-[1,1′-biphenyl]-3-yl)-3-(((((S)-5-oxopyrrolidin-2-yl)methyl)amino)methyl)-4H-pyrido[1,2-a]pyrimidin-4-one;
(S)-2-(((8-(2,2′-dichloro-3′-(6-methoxy-5-((((5-oxopyrrolidin-2-yl)methyl)amino)methyl)pyridin-2-yl)-[1,1′-biphenyl]-3-yl)-4-oxo-4H-pyrido[1,2-a]pyrimidin-3-yl)methyl)amino)-N,2-dimethylpropanamide;
(S)-3-(((8-(2,2′-dichloro-3′-(6-methoxy-5-((((5-oxopyrrolidin-2-yl)methyl)amino)methyl)pyridin-2-yl)-[1,1′-biphenyl]-3-yl)-4-oxo-4H-pyrido[1,2-a]pyrimidin-3-yl)methyl)amino)-N-methylpropanamide;
8-(2,2′-dichloro-3′-(6-methoxy-5-(((((S)-5-oxopyrrolidin-2-yl)methyl)amino)methyl)pyridin-2-yl)-[1,1′-biphenyl]-3-yl)-3-((((1r,3r)-3-hydroxy-3-methylcyclobutyl)amino)methyl)-6-methyl-4H-pyrido[1,2-a]pyrimidin-4-one;
7-(2,2′-dichloro-3′-(6-methoxy-5-(((((S)-5-oxopyrrolidin-2-yl)methyl)amino)methyl)pyridin-2-yl)-[1,1′-biphenyl]-3-yl)-2-(((((S)-5-oxopyrrolidin-2-yl)methyl)amino)methyl)-4H-pyrido[1,2-a]pyrimidin-4-one;
8-(2-chloro-3-(1-((3-chloro-6-methoxy-5-(((((S)-5-oxopyrrolidin-2-yl)methyl)amino)methyl)pyridin-2-yl)methyl)-2,3-dihydro-1H-inden-4-yl)phenyl)-3-(((((S)-5-oxopyrrolidin-2-yl)methyl)amino)methyl)-4H-pyrido[1,2-a]pyrimidin-4-one;
8-(2,2′-dichloro-3′-(6-methoxy-5-(((((S)-5-oxopyrrolidin-2-yl)methyl)amino)methyl)pyridin-2-yl)-[1,1′-biphenyl]-3-yl)-3-(((2-((S)-5-oxopyrrolidin-2-yl)ethyl)amino)methyl)-4H-pyrido[1,2-a]pyrimidin-4-one;
(S)-3-(((2-(1H-imidazol-4-yl)ethyl)amino)methyl)-8-(2,2′-dichloro-3′-(6-methoxy-5-((((5-oxopyrrolidin-2-yl)methyl)amino)methyl)pyridin-2-yl)-[1,1′-biphenyl]-3-yl)-4H-pyrido[1,2-a]pyrimidin-4-one;
(S)-3-(((2-(4H-1,2,4-triazol-4-yl)ethyl)amino)methyl)-8-(2,2′-dichloro-3′-(6-methoxy-5-((((5-oxopyrrolidin-2-yl)methyl)amino)methyl)pyridin-2-yl)-[1,1′-biphenyl]-3-yl)-4H-pyrido[1,2-a]pyrimidin-4-one;
(S)-5-((((6-(3′-(3-amino-2-(hydroxymethyl)thieno[3,2-b]pyridin-6-yl)-2,2′-dichloro-[1,1′-biphenyl]-3-yl)-2-methoxypyridin-3-yl)methyl)amino)methyl)pyrrolidin-2-one;
(S)-5-((((6-(2,2′-dichloro-3′-(2,3,4,5-tetrahydropyrido[3,2-f][1,4]oxazepin-8-yl)-[1,1′-biphenyl]-3-yl)-2-methoxypyridin-3-yl)methyl)amino)methyl)pyrrolidin-2-one;
(S)-8-(2,2′-dichloro-3′-(6-methoxy-5-((((5-oxopyrrolidin-2-yl)methyl)amino)methyl)pyridin-2-yl)-[1,1′-biphenyl]-3-yl)-3-(((2-(2-oxopyrrolidin-1-yl)ethyl)amino)methyl)-4H-pyrido[1,2-a]pyrimidin-4-one;
methyl (S)-4-(((8-(2,2′-dichloro-3′-(6-methoxy-5-(((((S)-5-oxopyrrolidin-2-yl)methyl)amino)methyl)pyridin-2-yl)-[1,1′-biphenyl]-3-yl)-4-oxo-4H-pyrido[1,2-a]pyrimidin-3-yl)methyl)amino)-3-hydroxybutanoate;
7-(2,2′-dichloro-3′-(6-methoxy-5-(((((S)-5-oxopyrrolidin-2-yl)methyl)amino)methyl)pyridin-2-yl)-[1,1′-biphenyl]-3-yl)-2-((((1r,3r)-3-hydroxycyclobutyl)amino)methyl)-4H-pyrido[1,2-a]pyrimidin-4-one;
(S)-7-(2,2′-dichloro-3′-(6-methoxy-5-((((5-oxopyrrolidin-2-yl)methyl)amino)methyl)pyridin-2-yl)-[1,1′-biphenyl]-3-yl)-2-((3-hydroxy-3-methylazetidin-1-yl)methyl)-4H-pyrido[1,2-a]pyrimidin-4-one;
(S)-8-(2,2′-dichloro-3′-(6-methoxy-5-((((5-oxopyrrolidin-2-yl)methyl)amino)methyl)pyridin-2-yl)-[1,1′-biphenyl]-3-yl)-3-(((4,4,5,5,5-pentafluoropentyl)amino)methyl)-4H-pyrido[1,2-a]pyrimidin-4-one;
(S)-8-(2,2′-dichloro-3′-(6-methoxy-5-((((5-oxopyrrolidin-2-yl)methyl)amino)methyl)pyridin-2-yl)-[1,1′-biphenyl]-3-yl)-3-(((1,1-dioxidotetrahydro-2H-thiopyran-4-yl)amino)methyl)-4H-pyrido[1,2-a]pyrimidin-4-one;
cis-8-(2,2′-dichloro-3′-(6-methoxy-5-(((((S)-5-oxopyrrolidin-2-yl)methyl)amino)methyl)pyridin-2-yl)-[1,1′-biphenyl]-3-yl)-3-((((2-(hydroxymethyl)cyclopropyl)methyl)amino)methyl)-4H-pyrido[1,2-a]pyrimidin-4-one;
8-(2,2′-dichloro-3′-(6-methoxy-5-(((((S)-5-oxopyrrolidin-2-yl)methyl)amino)methyl)pyridin-2-yl)-[1,1′-biphenyl]-3-yl)-3-((((S)-2-hydroxypropyl)amino)methyl)-6-methyl-4H-pyrido[1,2-a]pyrimidin-4-one;
2-chloro-8-(2,2′-dichloro-3′-(6-methoxy-5-(((((S)-5-oxopyrrolidin-2-yl)methyl)amino)methyl)pyridin-2-yl)-[1,1′-biphenyl]-3-yl)-3-(((((S)-5-oxopyrrolidin-2-yl)methyl)amino)methyl)-4H-pyrido[1,2-a]pyrimidin-4-one;
(S)-5-((((6-(2,2′-dichloro-3′-((6-methoxy-5-(((((S)-5-oxopyrrolidin-2-yl)methyl)amino)methyl)-2,3-dihydro-1H-inden-1-ylidene)methyl)-[1,1′-biphenyl]-3-yl)-2-methoxypyridin-3-yl)methyl)amino)methyl)pyrrolidin-2-one;
8-(2,2′-dichloro-3′-(6-methoxy-5-(((((S)-5-oxopyrrolidin-2-yl)methyl)amino)methyl)pyridin-2-yl)-[1,1′-biphenyl]-3-yl)-3-((((1s,3s)-3-hydroxy-3-methylcyclobutyl)amino)methyl)-2-methyl-4H-pyrido[1,2-a]pyrimidin-4-one;
8-(2,2′-dichloro-3′-(5-((((1s,3s)-3-hydroxy-3-methylcyclobutyl)amino)methyl)-6-methoxypyridin-2-yl)-[1,1′-biphenyl]-3-yl)-3-((((1s,3s)-3-hydroxy-3-methylcyclobutyl)amino)methyl)-2-methyl-4H-pyrido[1,2-a]pyrimidin-4-one;
methyl (S)-4-(((6-(2,2′-dichloro-3′-(3-((((S)-2-hydroxy-4-methoxy-4-oxobutyl)amino)methyl)-4-oxo-4H-pyrido[1,2-a]pyrimidin-8-yl)-[1,1′-biphenyl]-3-yl)-2-methoxypyridin-3-yl)methyl)amino)-3-hydroxybutanoate;
(S)-5-((((6-(2,2′-dichloro-3′-(4-fluoro-6-methoxy-5-(((((S)-5-oxopyrrolidin-2-yl)methyl)amino)methyl)pyridin-2-yl)-[1,1′-biphenyl]-3-yl)-2-methoxypyridin-3-yl)methyl)amino)methyl)pyrrolidin-2-one;
(S)-8-(2,2′-dichloro-3′-(6-methoxy-5-((((5-oxopyrrolidin-2-yl)methyl)amino)methyl)pyridin-2-yl)-[1,1′-biphenyl]-3-yl)-3-((((1-hydroxycyclobutyl)methyl)amino)methyl)-4H-pyrido[1,2-a]pyrimidin-4-one;
(S)-8-(2,2′-dichloro-3′-(6-methoxy-5-((((5-oxopyrrolidin-2-yl)methyl)amino)methyl)pyridin-2-yl)-[1,1′-biphenyl]-3-yl)-3-((((1-hydroxycyclopropyl)methyl)amino)methyl)-4H-pyrido[1,2-a]pyrimidin-4-one;
(5S)-5-((((6-(2,2′-dichloro-3′-(5-((((S)-5-oxopyrrolidin-2-yl)methyl)amino)-5,6,7,8-tetrahydroquinolin-2-yl)-[1,1′-biphenyl]-3-yl)-2-methoxypyridin-3-yl)methyl)amino)methyl)pyrrolidin-2-one;
(5S,5'S)-5,5′-(((((2,2′-dichloro-[1,1′-biphenyl]-3,3′-diyl)bis(4-fluoro-2-methoxypyridine-6,3-diyl))bis(methylene))bis(azanediyl))bis(methylene))bis(pyrrolidin-2-one);
(S)-8-(4′-((1-aminocyclopropyl)methyl)-2,2′-dichloro-[1,1′:3′,1″-terphenyl]-3-yl)-3-((((5-oxopyrrolidin-2-yl)methyl)amino)methyl)-4H-pyrido[1,2-a]pyrimidin-4-one;
(S)-4-(((8-(2,2′-dichloro-3′-(6-methoxy-5-(((((S)-5-oxopyrrolidin-2-yl)methyl)amino)methyl)pyridin-2-yl)-[1,1′-biphenyl]-3-yl)-4-oxo-4H-pyrido[1,2-a]pyrimidin-3-yl)methyl)amino)-3-hydroxybutanamide;
(S)-6-(2,2′-dichloro-3′-(6-methoxy-5-((((5-oxopyrrolidin-2-yl)methyl)amino)methyl)pyridin-2-yl)-[1,1′-biphenyl]-3-yl)-2-(2-hydroxyethyl)-[1,2,4]triazolo[4,3-a]pyridin-3(2H)-one;
(S)-5-((((6-(2,2′-dichloro-3′-(5,6,7,8-tetrahydro-[1,2,4]triazolo[4,3-a]pyrazin-3-yl)-[1,1′-biphenyl]-3-yl)-2-methoxypyridin-3-yl)methyl)amino)methyl)pyrrolidin-2-one;
(S)-5-((((6-(2,2′-dichloro-3′-(2-(4-(2-hydroxyethyl)piperazin-1-yl)imidazo[2,1-b][1,3,4]thiadiazol-5-yl)-[1,1′-biphenyl]-3-yl)-2-methoxypyridin-3-yl)methyl)amino)methyl)pyrrolidin-2-one;
(S)-5-((((6-(2,2′-dichloro-3′-(7-(2-hydroxyethyl)-5,6,7,8-tetrahydro-[1,2,4]triazolo[4,3-30a]pyrazin-3-yl)-[1,1′-biphenyl]-3-yl)-2-methoxypyridin-3-yl)methyl)amino)methyl)pyrrolidin-2-one;
(S)-8-(2,2′-dichloro-3′-(6-methoxy-5-((((5-oxopyrrolidin-2-yl)methyl)amino)methyl)pyridin-2-yl)-[1,1′-biphenyl]-3-yl)-3-((((3-hydroxyoxetan-3-yl)methyl)amino)methyl)-4H-pyrido[1,2-a]pyrimidin-4-one;
(S)-8-(2,2′-dichloro-3′-(6-methoxy-5-((((5-oxopyrrolidin-2-yl)methyl)amino)methyl)pyridin-2-yl)-[1,1′-biphenyl]-3-yl)-3-((((3-(hydroxymethyl)oxetan-3-yl)methyl)amino)methyl)-4H-pyrido[1,2-a]pyrimidin-4-one;
(S)-8-(2,2′-dichloro-3′-(6-methoxy-5-((((5-oxopyrrolidin-2-yl)methyl)amino)methyl)pyridin-2-yl)-[1,1′-biphenyl]-3-yl)-3-((((3-methyloxetan-3-yl)methyl)amino)methyl)-4H-pyrido[1,2-a]pyrimidin-4-one;
(S)-5-((((6-(2,2′-dichloro-3′-(2-(((((S)-5-oxopyrrolidin-2-yl)methyl)amino)methyl)benzo[d]oxazol-5-yl)-[1,1′-biphenyl]-3-yl)-2-methoxypyridin-3-yl)methyl)amino)methyl)pyrrolidin-2-one;
(S)-5-((((6-(2,2′-dichloro-3′-(2-(((((S)-5-oxopyrrolidin-2-yl)methyl)amino)methyl)benzo[d]oxazol-6-yl)-[1,1′-biphenyl]-3-yl)-2-methoxypyridin-3-yl)methyl)amino)methyl)pyrrolidin-2-one;
(S)-8-(2,2′-dichloro-3′-(6-methoxy-5-((((5-oxopyrrolidin-2-yl)methyl)amino)methyl)pyridin-2-yl)-[1,1′-biphenyl]-3-yl)-3-(((5-oxo-6-azaspiro[3.4]octan-2-yl)amino)methyl)-4H-pyrido[1,2-a]pyrimidin-4-one;
8-(2,2′-dichloro-3′-(6-methoxy-5-(((((S)-5-oxopyrrolidin-2-yl)methyl)amino)methyl)pyridin-2-yl)-[1,1′-biphenyl]-3-yl)-3-((((3-oxo-2-azabicyclo[2.2.1]heptan-1-yl)methyl)amino)methyl)-4H-pyrido[1,2-a]pyrimidin-4-one;
8-(2,2′-dichloro-3′-(6-methoxy-5-(((((S)-5-oxopyrrolidin-2-yl)methyl)amino)methyl)pyridin-2-yl)-[1,1′-biphenyl]-3-yl)-3-((((4,4-difluoro-5-oxopyrrolidin-2-yl)methyl)amino)methyl)-4H-pyrido[1,2-a]pyrimidin-4-one;
8-(2,2′-dichloro-3′-(6-methoxy-5-(((((S)-5-oxopyrrolidin-2-yl)methyl)amino)methyl)pyridin-2-yl)-[1,1′-biphenyl]-3-yl)-3-((((1s,3s)-3-hydroxy-3-methylcyclobutyl)amino)methyl)-6-methyl-4H-pyrido[1,2-a]pyrimidin-4-one;
(S)-1-((8-(2,2′-dichloro-3′-(6-methoxy-5-((((5-oxopyrrolidin-2-yl)methyl)amino)methyl)pyridin-2-yl)-[1,1′-biphenyl]-3-yl)-4-oxo-4H-pyrido[1,2-a]pyrimidin-3-yl)methyl)-N-methylazetidine-3-carboxamide;
(S)-3-(((1-acetylpiperidin-4-yl)amino)methyl)-8-(2,2′-dichloro-3′-(6-methoxy-5-((((5-oxopyrrolidin-2-yl)methyl)amino)methyl)pyridin-2-yl)-[1,1′-biphenyl]-3-yl)-4H-pyrido[1,2-a]pyrimidin-4-one;
(S)-1-((8-(2,2′-dichloro-3′-(6-methoxy-5-(((((S)-5-oxopyrrolidin-2-yl)methyl)amino)methyl)pyridin-2-yl)-[1,1′-biphenyl]-3-yl)-4-oxo-4H-pyrido[1,2-a]pyrimidin-3-yl)methyl)-N-methylpyrrolidine-3-carboxamide;
(S)-5-((((6-(2,2′-dichloro-3′-(3-(((((S)-5-oxopyrrolidin-2-yl)methyl)amino)methyl)-1,6-naphthyridin-7-yl)-[1,1′-biphenyl]-3-yl)-2-methoxypyridin-3-yl)methyl)amino)methyl)pyrrolidin-2-one;
1-((8-(2,2′-dichloro-3′-(6-methoxy-5-(((((S)-5-oxopyrrolidin-2-yl)methyl)amino)methyl)pyridin-2-yl)-[1,1′-biphenyl]-3-yl)-4-oxo-4H-pyrido[1,2-a]pyrimidin-3-yl)methyl)azetidine-2-carboxamide;
(S)-2-(((8-(2,2′-dichloro-3′-(6-methoxy-5-(((((S)-5-oxopyrrolidin-2-yl)methyl)amino)methyl)pyridin-2-yl)-[1,1′-biphenyl]-3-yl)-4-oxo-4H-pyrido[1,2-a]pyrimidin-3-yl)methyl)amino)propanamide;
(S)-8-(2,2′-dichloro-3′-(2,3,4,5-tetrahydropyrido[3,2-f][1,4]oxazepin-8-yl)-[1,1′-biphenyl]-3-yl)-3-((((5-oxopyrrolidin-2-yl)methyl)amino)methyl)-4H-pyrido[1,2-a]pyrimidin-4-one;
8-(2,2′-dichloro-3′-(6-methoxy-5-(((((S)-5-oxopyrrolidin-2-yl)methyl)amino)methyl)pyridin-2-yl)-[1,1′-biphenyl]-3-yl)-3-((((S)-2-hydroxypropyl)amino)methyl)-2-methoxy-4H-pyrido[1,2-a]pyrimidin-4-one;
(S)-2-(((8-(2,2′-dichloro-3′-(6-methoxy-5-(((((S)-5-oxopyrrolidin-2-yl)methyl)amino)methyl)pyridin-2-yl)-[1,1′-biphenyl]-3-yl)-4-oxo-4H-pyrido[1,2-a]pyrimidin-3-yl)methyl)(methyl)amino)-N-methylpropanamide;
(S)-8-(2,2′-dichloro-3′-(6-methoxy-5-((((5-oxopyrrolidin-2-yl)methyl)amino)methyl)pyridin-2-yl)-[1,1′-biphenyl]-3-yl)-3-(((2-(3-oxopiperazin-1-yl)ethyl)amino)methyl)-4H-pyrido[1,2-a]pyrimidin-4-one;
methyl (S)-1-((8-(2,2′-dichloro-3′-(6-methoxy-5-(((((S)-5-oxopyrrolidin-2-yl)methyl)amino)methyl)pyridin-2-yl)-[1,1′-biphenyl]-3-yl)-2-methyl-4-oxo-4H-pyrido[1,2-a]pyrimidin-3-yl)methyl)pyrrolidine-3-carboxylate;
(S)-8-(2,2′-dichloro-3′-(6-methoxy-5-((((5-oxopyrrolidin-2-yl)methyl)amino)methyl)pyridin-2-yl)-[1,1′-biphenyl]-3-yl)-3-(((2-(1,1-dioxidothiomorpholino)ethyl)amino)methyl)-4H-pyrido[1,2-a]pyrimidin-4-one;
(S)-5-((((6-(2,2′-dichloro-3′-(4-(2-hydroxyethyl)-2,3,4,5-tetrahydropyrido[3,2-f1][1,4]oxazepin-8-yl)-[1,1′-biphenyl]-3-yl)-2-methoxypyridin-3-yl)methyl)amino)methyl)pyrrolidin-2-one;
(S)-8-(2,2′-dichloro-3′-(4-(2-hydroxyethyl)-2,3,4,5-tetrahydropyrido[3,2-f][1,4]oxazepin-8-yl)-[1,1′-biphenyl]-3-yl)-3-((((5-oxopyrrolidin-2-yl)methyl)amino)methyl)-4H-pyrido[1,2-a]pyrimidin-4-one;
methyl (S)-((8-(2,2′-dichloro-3′-(6-methoxy-5-((((5-oxopyrrolidin-2-yl)methyl)amino)methyl)pyridin-2-yl)-[1,1′-biphenyl]-3-yl)-2-methyl-4-oxo-4H-pyrido[1,2-a]pyrimidin-3-yl)methyl)glycinate;
(S)-4-(((8-(2,2′-dichloro-3′-(4-fluoro-6-methoxy-5-(((((S)-5-oxopyrrolidin-2-yl)methyl)amino)methyl)pyridin-2-yl)-[1,1′-biphenyl]-3-yl)-4-oxo-4H-pyrido[1,2-a]pyrimidin-3-yl)methyl)amino)-3-hydroxybutanoic acid;
methyl (S)-4-(((8-(2,2′-dichloro-3′-(4-fluoro-6-methoxy-5-(((((S)-5-oxopyrrolidin-2-yl)methyl)amino)methyl)pyridin-2-yl)-[1,1′-biphenyl]-3-yl)-4-oxo-4H-pyrido[1,2-a]pyrimidin-3-yl)methyl)amino)-3-hydroxybutanoate;
8-(2,2′-dichloro-3′-(4-fluoro-6-methoxy-5-(((((S)-5-oxopyrrolidin-2-yl)methyl)amino)methyl)pyridin-2-yl)-[1,1′-biphenyl]-3-yl)-3-((((S)-2-hydroxypropyl)amino)methyl)-4H-pyrido[1,2-a]pyrimidin-4-one;
(S)-8-(2,2′-dichloro-3′-(6-methoxy-5-((((5-oxopyrrolidin-2-yl)methyl)amino)methyl)pyridin-2-yl)-[1,1′-biphenyl]-3-yl)-3-(((3-hydroxy-2,2-dimethylpropyl)amino)methyl)-4H-pyrido[1,2-a]pyrimidin-4-one;
3-(((2-cyclopropyl-2-hydroxyethyl)amino)methyl)-8-(2,2′-dichloro-3′-(6-methoxy-5-(((((S)-5-oxopyrrolidin-2-yl)methyl)amino)methyl)pyridin-2-yl)-[1,1′-biphenyl]-3-yl)-4H-pyrido[1,2-a]pyrimidin-4-one;
(S)-4-(((8-(2,2′-dichloro-3′-(4-fluoro-6-methoxy-5-(((((S)-5-oxopyrrolidin-2-yl)methyl)amino)methyl)pyridin-2-yl)-[1,1′-biphenyl]-3-yl)-4-oxo-4H-pyrido[1,2-a]pyrimidin-3-yl)methyl)amino)-3-hydroxybutanamide;
8-(2,2′-dichloro-3′-(6-methoxy-5-((methyl(3-(methylamino)propyl)amino)methyl)pyridin-2-yl)-[1,1′-biphenyl]-3-yl)-3-((methyl(2-(methylamino)ethyl)amino)methyl)-4H-pyrido[1,2-a]pyrimidin-4-one;
tert-butyl (2-(((6-(3′-(3-(((2-((tert-butoxycarbonyl)amino)ethyl)(methyl)amino)methyl)-4-oxo-4H-pyrido[1,2-a]pyrimidin-8-yl)-2,2′-dichloro-[1,1′-biphenyl]-3-yl)-2-methoxypyridin-3-yl)methyl)(methyl)amino)ethyl)carbamate;
(S)-8-(2,2′-dichloro-3′-(6-methoxy-5-((((5-oxopyrrolidin-2-yl)methyl)amino)methyl)pyridin-2-yl)-[1,1′-biphenyl]-3-yl)-3-((methyl(2-(methylamino)ethyl)amino)methyl)-4H-pyrido[1,2-a]pyrimidin-4-one;
(S)-3-(((2-aminoethyl)(methyl)amino)methyl)-8-(2,2′-dichloro-3′-(6-methoxy-5-((((5-oxopyrrolidin-2-yl)methyl)amino)methyl)pyridin-2-yl)-[1,1′-biphenyl]-3-yl)-4H-pyrido[1,2-a]pyrimidin-4-one;
3-(((2-cyclopropyl-2-hydroxypropyl)amino)methyl)-8-(2,2′-dichloro-3′-(6-methoxy-5-(((((S)-5-oxopyrrolidin-2-yl)methyl)amino)methyl)pyridin-2-yl)-[1,1′-biphenyl]-3-yl)-4H-pyrido[1,2-a]pyrimidin-4-one;
(S)-8-(2,2′-dichloro-3′-(6-methoxy-5-((((5-oxopyrrolidin-2-yl)methyl)amino)methyl)pyridin-2-yl)-[1,1′-biphenyl]-3-yl)-3-(((2-hydroxyethyl)(methyl)amino)methyl)-4H-pyrido[1,2-a]pyrimidin-4-one;
(S)-5-((((6-(2,2′-dichloro-3′-(3-(((((S)-5-oxopyrrolidin-2-yl)methyl)amino)methyl)pyrazolo[1,5-a]pyrimidin-5-yl)-[1,1′-biphenyl]-3-yl)-2-methoxypyridin-3-yl)methyl)amino)methyl)pyrrolidin-2-one;
(S)-8-(3′-(5-(1-(aminomethyl)cyclopropyl)-6-methoxypyridin-2-yl)-2,2′-dichloro-[1,1′-biphenyl]-3-yl)-3-((((5-oxopyrrolidin-2-yl)methyl)amino)methyl)-4H-pyrido[1,2-a]pyrimidin-4-one;
8-(2,2′-dichloro-3′-(6-methoxy-5-(((((S)-5-oxopyrrolidin-2-yl)methyl)amino)methyl)pyridin-2-yl)-[1,1′-biphenyl]-3-yl)-3-(((((S)-5-oxopyrrolidin-3-yl)methyl)amino)methyl)-4H-pyrido[1,2-a]pyrimidin-4-one;
8-(2,2′-dichloro-3′-(6-methoxy-5-(((((S)-5-oxopyrrolidin-2-yl)methyl)amino)methyl)pyridin-2-yl)-[1,1′-biphenyl]-3-yl)-3-((((3R,4S)-3-hydroxytetrahydro-2H-pyran-4-yl)amino)methyl)-2-methyl-4H-pyrido[1,2-a]pyrimidin-4-one;
8-(2,2′-dichloro-3′-(5-((((3R)-3-hydroxytetrahydro-2H-pyran-4-yl)amino)methyl)-6-methoxypyridin-2-yl)-[1,1′-biphenyl]-3-yl)-2-methyl-3-(((((S)-5-oxopyrrolidin-2-yl)methyl)amino)methyl)-4H-pyrido[1,2-a]pyrimidin-4-one;
(S)-8-(2,2′-dichloro-3′-(6-methoxy-5-((((5-oxopyrrolidin-2-yl)methyl)amino)methyl)pyridin-2-yl)-[1,1′-biphenyl]-3-yl)-3-(((2-hydroxyethyl)amino)methyl)-2-methyl-4H-pyrido[1,2-a]pyrimidin-4-one;
7-(2,2′-dichloro-3′-(6-methoxy-5-(((((S)-5-oxopyrrolidin-2-yl)methyl)amino)methyl)pyridin-2-yl)-[1,1′-biphenyl]-3-yl)-9-methyl-2-(((((S)-5-oxopyrrolidin-2-yl)methyl)amino)methyl)-4H-pyrido[1,2-a]pyrimidin-4-one;
methyl 1-((6-(2,2′-dichloro-3′-(3-((3-(methoxycarbonyl)azetidin-1-yl)methyl)-1,6-naphthyridin-7-yl)-[1,1′-biphenyl]-3-yl)-2-methoxypyridin-3-yl)methyl)azetidine-3-carboxylate;
8-(2,2′-dichloro-3′-(6-methoxy-5-(((((S)-5-oxopyrrolidin-2-yl)methyl)amino)methyl)pyridin-2-yl)-[1,1′-biphenyl]-3-yl)-3-(5-hydroxy-1,4,5,6-tetrahydropyrimidin-2-yl)-2-methyl-4H-pyrido[1,2-a]pyrimidin-4-one;
methyl (S)-1-((8-(2,2′-dichloro-3′-(6-methoxy-5-((((5-oxopyrrolidin-2-yl)methyl)amino)methyl)pyridin-2-yl)-[1,1′-biphenyl]-3-yl)-6-methyl-4-oxo-4H-pyrido[1,2-a]pyrimidin-3-yl)methyl)azetidine-3-carboxylate;
(S)-4-(((8-(2,2′-dichloro-3′-(6-methoxy-5-(((((S)-5-oxopyrrolidin-2-yl)methyl)amino)methyl)pyridin-2-yl)-[1,1′-biphenyl]-3-yl)-4-oxo-4H-pyrido[1,2-a]pyrimidin-3-yl)methyl)amino)-3-hydroxy-N-methylbutanamide;
(S)-4-(((8-(2,2′-dichloro-3′-(4-fluoro-6-methoxy-5-(((((S)-5-oxopyrrolidin-2-yl)methyl)amino)methyl)pyridin-2-yl)-[1,1′-biphenyl]-3-yl)-4-oxo-4H-pyrido[1,2-a]pyrimidin-3-yl)methyl)amino)-3-hydroxy-N-methylbutanamide;
methyl (S)-((8-(2,2′-dichloro-3′-(6-methoxy-5-((((5-oxopyrrolidin-2-yl)methyl)amino)methyl)pyridin-2-yl)-[1,1′-biphenyl]-3-yl)-6-methyl-4-oxo-4H-pyrido[1,2-a]pyrimidin-3-yl)methyl)glycinate;
methyl (S)-1-((8-(2,2′-dichloro-3′-(6-methoxy-5-(((((S)-5-oxopyrrolidin-2-yl)methyl)amino)methyl)pyridin-2-yl)-[1,1′-biphenyl]-3-yl)-6-methyl-4-oxo-4H-pyrido[1,2-a]pyrimidin-3-yl)methyl)pyrrolidine-3-carboxylate;
methyl (S)-1-((8-(2,2′-dichloro-3′-(6-methoxy-5-((((5-oxopyrrolidin-2-yl)methyl)amino)methyl)pyridin-2-yl)-[1,1′-biphenyl]-3-yl)-2-methyl-4-oxo-4H-pyrido[1,2-a]pyrimidin-3-yl)methyl)azetidine-3-carboxylate;
(S)-8-(2,2′-dichloro-3′-(6-methoxy-5-((((5-oxopyrrolidin-2-yl)methyl)amino)methyl)pyridin-2-yl)-[1,1′-biphenyl]-3-yl)-3-(((2,2-difluoroethyl)amino)methyl)-4H-pyrido[1,2-a]pyrimidin-4-one;
3-((((S)-2-aminopropyl)amino)methyl)-8-(2,2′-dichloro-3′-(6-methoxy-5-(((((S)-5-oxopyrrolidin-2-yl)methyl)amino)methyl)pyridin-2-yl)-[1,1′-biphenyl]-3-yl)-4H-pyrido[1,2-a]pyrimidin-4-one;
methyl (2-(((8-(2,2′-dichloro-3′-(6-methoxy-5-(((2-((methoxycarbonyl)(methyl)amino)ethyl)(methyl)amino)methyl)pyridin-2-yl)-[1,1′-biphenyl]-3-yl)-4-oxo-4H-pyrido[1,2-a]pyrimidin-3-yl)methyl)(methyl)amino)ethyl)(methyl)carbamate;
(S)-8-(2,2′-dichloro-3′-(6-methoxy-5-((((5-oxopyrrolidin-2-yl)methyl)amino)methyl)pyridin-2-yl)-[1,1′-biphenyl]-3-yl)-3-(((2,2-difluoro-3-hydroxypropyl)amino)methyl)-4H-pyrido[1,2-a]pyrimidin-4-one;
(S)-8-(2,2′-dichloro-3′-(6-methoxy-5-((((5-oxopyrrolidin-2-yl)methyl)amino)methyl)pyridin-2-yl)-[1,1′-biphenyl]-3-yl)-3-((((1-(hydroxymethyl)cyclopropyl)methyl)amino)methyl)-4H-pyrido[1,2-a]pyrimidin-4-one;
8-(2,2′-dichloro-3′-(6-methoxy-5-(((((S)-5-oxopyrrolidin-2-yl)methyl)amino)methyl)pyridin-2-yl)-[1,1′-biphenyl]-3-yl)-3-((((S)-2-methoxypropyl)amino)methyl)-4H-pyrido[1,2-a]pyrimidin-4-one;
(S)-8-(2,2′-dichloro-3′-(6-methoxy-5-((((5-oxopyrrolidin-2-yl)methyl)amino)methyl)pyridin-2-yl)-[1,1′-biphenyl]-3-yl)-3-(((2,2,2-trifluoroethyl)amino)methyl)-4H-pyrido[1,2-a]pyrimidin-4-one;
methyl (S)-1-((7-(2,2′-dichloro-3′-(6-methoxy-5-((((5-oxopyrrolidin-2-yl)methyl)amino)methyl)pyridin-2-yl)-[1,1′-biphenyl]-3-yl)-1,6-naphthyridin-3-yl)methyl)azetidine-3-carboxylate;
methyl (S)-((7-(2,2′-dichloro-3′-(6-methoxy-5-((((5-oxopyrrolidin-2-yl)methyl)amino)methyl)pyridin-2-yl)-[1,1′-biphenyl]-3-yl)-1,6-naphthyridin-3-yl)methyl)glycinate;
8-(2,2′-dichloro-3′-(6-methoxy-5-(((((S)-5-oxopyrrolidin-2-yl)methyl)amino)methyl)pyridin-2-yl)-[1,1′-biphenyl]-3-yl)-3-(((((S)-oxetan-2-yl)methyl)amino)methyl)-4H-pyrido[1,2-a]pyrimidin-4-one;
8-(2,2′-dichloro-3′-(6-methoxy-5-(((((S)-5-oxopyrrolidin-2-yl)methyl)amino)methyl)pyridin-2-yl)-[1,1′-biphenyl]-3-yl)-3-(((((S)-tetrahydrofuran-2-yl)methyl)amino)methyl)-4H-pyrido[1,2-a]pyrimidin-4-one;
(S)-8-(2,2′-dichloro-3′-(6-methoxy-5-((((5-oxopyrrolidin-2-yl)methyl)amino)methyl)pyridin-2-yl)-[1,1′-biphenyl]-3-yl)-3-(((2-hydroxyethyl)amino)methyl)-6-methyl-4H-pyrido[1,2-a]pyrimidin-4-one;
(S)-5-((((6-(2,2′-dichloro-3′-(3-(((((S)-5-oxopyrrolidin-2-yl)methyl)amino)methyl)imidazo[1,2-a]pyrazin-6-yl)-[1,1′-biphenyl]-3-yl)-2-methoxypyridin-3-yl)methyl)amino)methyl)pyrrolidin-2-one;
(S)-5-((((6-(2,2′-dichloro-3′-(3-(((((S)-5-oxopyrrolidin-2-yl)methyl)amino)methyl)pyrazolo[1,5-a]pyrimidin-6-yl)-[1,1′-biphenyl]-3-yl)-2-methoxypyridin-3-yl)methyl)amino)methyl)pyrrolidin-2-one;
(S)-5-((((6-(2,2′-dichloro-3′-(2-(((((S)-5-oxopyrrolidin-2-yl)methyl)amino)methyl)furo[3,2-b]pyridin-6-yl)-[1,1′-biphenyl]-3-yl)-2-methoxypyridin-3-yl)methyl)amino)methyl)pyrrolidin-2-one;
(S)-5-((((6-(2,2′-dichloro-3′-(1-methyl-2-(((((S)-5-oxopyrrolidin-2-yl)methyl)amino)methyl)-1H-imidazo[4,5-b]pyridin-5-yl)-[1,1′-biphenyl]-3-yl)-2-methoxypyridin-3-yl)methyl)amino)methyl)pyrrolidin-2-one;
(1r,3r)-3-(((6-(2,2′-dichloro-3′-(2-((((1r,3r)-3-hydroxycyclobutyl)amino)methyl)-1-methyl-1H-imidazo[4,5-b]pyridin-5-yl)-[1,1′-biphenyl]-3-yl)-2-methoxypyridin-3-yl)methyl)amino)cyclobutan-1-ol;
(S)-5-((((6-(2,2′-dichloro-3′-(3-methyl-2-(((((S)-5-oxopyrrolidin-2-yl)methyl)amino)methyl)-3H-imidazo[4,5-b]pyridin-5-yl)-[1,1′-biphenyl]-3-yl)-2-methoxypyridin-3-yl)methyl)amino)methyl)pyrrolidin-2-one;
(1r,3r)-3-(((6-(2,2′-dichloro-3′-(2-((((1r,3r)-3-hydroxycyclobutyl)amino)methyl)-3-methyl-3H-imidazo[4,5-b]pyridin-5-yl)-[1,1′-biphenyl]-3-yl)-2-methoxypyridin-3-yl)methyl)amino)cyclobutan-1-ol;
(S)-8-(2,2′-dichloro-3′-(6-methoxy-5-((((5-oxopyrrolidin-2-yl)methyl)amino)methyl)pyridin-2-yl)-[1,1′-biphenyl]-3-yl)-3-(((3-hydroxypropyl)amino)methyl)-4H-pyrido[1,2-a]pyrimidin-4-one;
(S)-8-(2,2′-dichloro-3′-(6-methoxy-5-((((5-oxopyrrolidin-2-yl)methyl)amino)methyl)pyridin-2-yl)-[1,1′-biphenyl]-3-yl)-3-(((3-hydroxypropyl)(methyl)amino)methyl)-4H-pyrido[1,2-a]pyrimidin-4-one;
8-(2,2′-dichloro-3′-(6-methoxy-5-(((((S)-5-oxopyrrolidin-2-yl)methyl)amino)methyl)pyridin-2-yl)-[1,1′-biphenyl]-3-yl)-3-((((1r,3r)-3-hydroxy-3-methylcyclobutyl)(methyl)amino)methyl)-4H-pyrido[1,2-a]pyrimidin-4-one;
8-(2,2′-dichloro-3′-(6-methoxy-5-(((((S)-5-oxopyrrolidin-2-yl)methyl)amino)methyl)pyridin-2-yl)-[1,1′-biphenyl]-3-yl)-3-((((S)-2-hydroxypropyl)(methyl)amino)methyl)-4H-pyrido[1,2-a]pyrimidin-4-one;
ethyl (S)-4-(((8-(2,2′-dichloro-3′-(6-methoxy-5-(((((S)-5-oxopyrrolidin-2-yl)methyl)amino)methyl)pyridin-2-yl)-[1,1′-biphenyl]-3-yl)-4-oxo-4H-pyrido[1,2-a]pyrimidin-3-yl)methyl)amino)-3-hydroxybutanoate;
8-(2,2′-dichloro-3′-(6-methoxy-5-(((2-(2-oxopyrrolidin-1-yl)ethyl)amino)methyl)pyridin-2-yl)-[1,1′-biphenyl]-3-yl)-3-(((2-(2-oxopyrrolidin-1-yl)ethyl)amino)methyl)-4H-pyrido[1,2-a]pyrimidin-4-one;
3-(((1-acetylpiperidin-4-yl)amino)methyl)-8-(3′-(5-(((1-acetylpiperidin-4-yl)amino)methyl)-6-methoxypyridin-2-yl)-2,2′-dichloro-[1,1′-biphenyl]-3-yl)-4H-pyrido[1,2-a]pyrimidin-4-one;
8-(2,2′-dichloro-3′-(6-methoxy-5-(((((S)-5-oxopyrrolidin-2-yl)methyl)amino)methyl)pyridin-2-yl)-[1,1′-biphenyl]-3-yl)-3-(5-hydroxy-1,4,5,6-tetrahydropyrimidin-2-yl)-6-methyl-4H-pyrido[1,2-a]pyrimidin-4-one;
7-(2,2′-dichloro-3′-(6-methoxy-5-(((((S)-5-oxopyrrolidin-2-yl)methyl)amino)methyl)pyridin-2-yl)-[1,1′-biphenyl]-3-yl)-2-((((S)-2-hydroxypropyl)amino)methyl)-4H-pyrido[1,2-a]pyrimidin-4-one;
7-(2,2′-dichloro-3′-(6-methoxy-5-(((((S)-5-oxopyrrolidin-2-yl)methyl)amino)methyl)pyridin-2-yl)-[1,1′-biphenyl]-3-yl)-2-((((1r,3r)-3-hydroxy-3-methylcyclobutyl)amino)methyl)-4H-pyrido[1,2-a]pyrimidin-4-one;
(5S)-5-((((6-(2,2′-dichloro-3′-(4-((((S)-5-oxopyrrolidin-2-yl)methyl)amino)chroman-7-yl)-[1,1′-biphenyl]-3-yl)-2-methoxypyridin-3-yl)methyl)amino)methyl)pyrrolidin-2-one;
(S)-8-(2,2′-dichloro-3′-(6-methoxy-5-((((5-oxopyrrolidin-2-yl)methyl)amino)methyl)pyridin-2-yl)-[1,1′-biphenyl]-3-yl)-3-((5-oxo-1,4-diazepan-1-yl)methyl)-4H-pyrido[1,2-a]pyrimidin-4-one;
(S)-3-((3,3-bis(hydroxymethyl)pyrrolidin-1-yl)methyl)-8-(2,2′-dichloro-3′-(6-methoxy-5-((((5-oxopyrrolidin-2-yl)methyl)amino)methyl)pyridin-2-yl)-[1,1′-biphenyl]-3-yl)-4H-pyrido[1,2-a]pyrimidin-4-one;
(S)-3-((6-acetyl-2,6-diazaspiro[3.3]heptan-2-yl)methyl)-8-(2,2′-dichloro-3′-(6-methoxy-5-((((5-oxopyrrolidin-2-yl)methyl)amino)methyl)pyridin-2-yl)-[1,1′-biphenyl]-3-yl)-4H-pyrido[1,2-a]pyrimidin-4-one;
N-((6-(2,2′-dichloro-3′-(6-methoxy-5-(((((S)-5-oxopyrrolidin-2-yl)methyl)amino)methyl)pyridin-2-yl)-[1,1′-biphenyl]-3-yl)-2-methoxypyridin-3-yl)methyl)-N—(((S)-5-oxopyrrolidin-2-yl)methyl)formamide;
6-(2,2′-dichloro-3′-(6-methoxy-5-((((5-oxopyrrolidin-2-yl)methyl)amino)methyl)pyridin-2-yl)-[1,1′-biphenyl]-3-yl)-3-methyl-2-(((((S)-5-oxopyrrolidin-2-yl)methyl)amino)methyl)pyrrolo[2,1-f][1,2,4]triazin-4(3H)-one;
6-(2,2′-dichloro-3′-(6-methoxy-5-(((((S)-5-oxopyrrolidin-2-yl)methyl)amino)methyl)pyridin-2-yl)-[1,1′-biphenyl]-3-yl)-2-((((1r,3r)-3-hydroxy-3-methylcyclobutyl)amino)methyl)-3-methylpyrrolo[2,1-f][1,2,4]triazin-4(3H)-one;
(S)-5-((((6-(2,2′-dichloro-3′-(3-((((S)-2-hydroxypropyl)amino)methyl)-1,6-naphthyridin-7-yl)-[1,1′-biphenyl]-3-yl)-2-methoxypyridin-3-yl)methyl)amino)methyl)pyrrolidin-2-one;
(S)-5-((((6-(2,2′-dichloro-3′-(3-(((2-hydroxyethyl)amino)methyl)-1,6-naphthyridin-7-yl)-[1,1′-biphenyl]-3-yl)-2-methoxypyridin-3-yl)methyl)amino)methyl)pyrrolidin-2-one;
8-(2,2′-dichloro-3′-(4-chloro-6-methoxy-5-(((((S)-5-oxopyrrolidin-2-yl)methyl)amino)methyl)pyridin-2-yl)-[1,1′-biphenyl]-3-yl)-3-(((((S)-5-oxopyrrolidin-2-yl)methyl)amino)methyl)-4H-pyrido[1,2-a]pyrimidin-4-one;
(S)-4-(((8-(2,2′-dichloro-3′-(4-fluoro-6-methoxy-5-(((((S)-5-oxopyrrolidin-2-yl)methyl)amino)methyl)pyridin-2-yl)-[1,1′-biphenyl]-3-yl)-4-oxo-4H-pyrido[1,2-a]pyrimidin-3-yl)methyl)amino)-3-hydroxy-N,N-dimethylbutanamide;
8-(2,2′-dichloro-3′-(4-chloro-6-methoxy-3-(((((R)-5-oxopyrrolidin-2-yl)methyl)amino)methyl)pyridin-2-yl)-[1,1′-biphenyl]-3-yl)-3-(((((S)-5-oxopyrrolidin-2-yl)methyl)amino)methyl)-4H-pyrido[1,2-a]pyrimidin-4-one;
(S)-5-((((6-(2,2′-dichloro-3′-(2-(((((S)-5-oxopyrrolidin-2-yl)methyl)amino)methyl)imidazo[1,2-b]pyridazin-6-yl)-[1,1′-biphenyl]-3-yl)-2-methoxypyridin-3-yl)methyl)amino)methyl)pyrrolidin-2-one;
2-(((6-(2,2′-dichloro-3′-(2-(((2-hydroxyethyl)(methyl)amino)methyl)imidazo[1,2-b]pyridazin-6-yl)-[1,1′-biphenyl]-3-yl)-2-methoxypyridin-3-yl)methyl)(methyl)amino)ethan-1-01;
(S)-1-(((6-(2,2′-dichloro-3′-(2-((((S)-2-hydroxypropyl)amino)methyl)imidazo[1,2-b]pyridazin-6-yl)-[1,1′-biphenyl]-3-yl)-2-methoxypyridin-3-yl)methyl)amino)propan-2-ol;
(S)-2-(((8-(2,2′-dichloro-3′-(6-methoxy-5-((((5-oxopyrrolidin-2-yl)methyl)amino)methyl)pyridin-2-yl)-[1,1′-biphenyl]-3-yl)-4-oxo-4H-pyrido[1,2-a]pyrimidin-2-yl)methyl)amino)-N,2-dimethylpropanamide;
8-(2,2′-dichloro-3′-(6-methoxy-5-(((((S)-5-oxopyrrolidin-2-yl)methyl)amino)methyl)pyridin-2-yl)-[1,1′-biphenyl]-3-yl)-2-(((S)-4-hydroxy-2-oxopyrrolidin-1-yl)methyl)-4H-pyrido[1,2-a]pyrimidin-4-one;
methyl (S)-1-((8-(2,2′-dichloro-3′-(6-methoxy-5-((((5-oxopyrrolidin-2-yl)methyl)amino)methyl)pyridin-2-yl)-[1,1′-biphenyl]-3-yl)-4-oxo-4H-pyrido[1,2-a]pyrimidin-2-yl)methyl)azetidine-3-carboxylate;
(S)-5-((((6-(2,2′-dichloro-3′-(2,3,4,5-tetrahydro-1H-benzo[c]azepin-7-yl)-[1,1′-biphenyl]-3-yl)-2-methoxypyridin-3-yl)methyl)amino)methyl)pyrrolidin-2-one;
(S)-8-(2,2′-dichloro-3′-(6-methoxy-5-((((5-oxopyrrolidin-2-yl)methyl)amino)methyl)pyridin-2-yl)-[1,1′-biphenyl]-3-yl)-3-(((2-methoxy-2-methylpropyl)amino)methyl)-4H-pyrido[1,2-a]pyrimidin-4-one;
(S)-8-(2,2′-dichloro-3′-(6-methoxy-5-((((5-oxopyrrolidin-2-yl)methyl)amino)methyl)pyridin-2-yl)-[1,1′-biphenyl]-3-yl)-3-((6-hydroxy-2-azaspiro[3.3]heptan-2-yl)methyl)-4H-pyrido[1,2-a]pyrimidin-4-one;
8-(2,2′-dichloro-3′-(6-methoxy-5-(((((S)-5-oxopyrrolidin-2-yl)methyl)amino)methyl)pyridin-2-yl)-[1,1′-biphenyl]-3-yl)-3-(((3R,4R)-3,4-dihydroxypyrrolidin-1-yl)methyl)-4H-pyrido[1,2-a]pyrimidin-4-one;
(S)-8-(2,2′-dichloro-3′-(6-methoxy-5-((((5-oxopyrrolidin-2-yl)methyl)amino)methyl)pyridin-2-yl)-[1,1′-biphenyl]-3-yl)-3-(((3-hydroxy-3-methylcyclobutyl)amino)methyl)-2-methoxy-4H-pyrido[1,2-a]pyrimidin-4-one;
(S)-1-(((8-(2,2′-dichloro-3′-(6-methoxy-5-((((5-oxopyrrolidin-2-yl)methyl)amino)methyl)pyridin-2-yl)-[1,1′-biphenyl]-3-yl)-4-oxo-4H-pyrido[1,2-a]pyrimidin-2-yl)methyl)amino)-N-methylcyclopropane-1-carboxamide;
6-(2,2′-dichloro-3′-(6-methoxy-5-(((((S)-5-oxopyrrolidin-2-yl)methyl)amino)methyl)pyridin-2-yl)-[1,1′-biphenyl]-3-yl)-2-((((S)-2-hydroxypropyl)amino)methyl)-3-methylpyrrolo[2,1-f][1,2,4]triazin-4(3H)-one;
(S)-6-(2,2′-dichloro-3′-(6-methoxy-5-((((5-oxopyrrolidin-2-yl)methyl)amino)methyl)pyridin-2-yl)-[1,1′-biphenyl]-3-yl)-2-(((2-hydroxyethyl)amino)methyl)-3-methylpyrrolo[2,1-f][1,2,4]triazin-4(3H)-one;
(S)-5-((((6-(2,2′-dichloro-3′-(2-(((2-hydroxyethyl)amino)methyl)imidazo[1,2-b]pyridazin-6-yl)-[1,1′-biphenyl]-3-yl)-2-methoxypyridin-3-yl)methyl)amino)methyl)pyrrolidin-2-one;
(S)-5-((((6-(2,2′-dichloro-3′-(2-(((2-hydroxyethyl)(methyl)amino)methyl)imidazo[1,2-b]pyridazin-6-yl)-[1,1′-biphenyl]-3-yl)-2-methoxypyridin-3-yl)methyl)amino)methyl)pyrrolidin-2-one;
(S)-5-((((6-(2,2′-dichloro-3′-(2-((((S)-2-hydroxypropyl)amino)methyl)imidazo[1,2-b]pyridazin-6-yl)-[1,1′-biphenyl]-3-yl)-2-methoxypyridin-3-yl)methyl)amino)methyl)pyrrolidin-2-one;
(S)-4-(((6-(2,2′-dichloro-3′-(6-methoxy-5-(((((S)-5-oxopyrrolidin-2-yl)methyl)amino)methyl)pyridin-2-yl)-[1,1′-biphenyl]-3-yl)imidazo[1,2-b]pyridazin-2-yl)methyl)amino)-3-hydroxy-N,N-dimethylbutanamide;
(S)-5-((((6-(2,2′-dichloro-3′-(2-(((((S)-5-oxopyrrolidin-2-yl)methyl)amino)methyl)imidazo[1,2-a]pyridin-6-yl)-[1,1′-biphenyl]-3-yl)-2-methoxypyridin-3-yl)methyl)amino)methyl)pyrrolidin-2-one;
(S)-5-((((6-(2,2′-dichloro-3′-(2-(((((S)-5-oxopyrrolidin-2-yl)methyl)amino)methyl)imidazo[1,2-a]pyridin-7-yl)-[1,1′-biphenyl]-3-yl)-2-methoxypyridin-3-yl)methyl)amino)methyl)pyrrolidin-2-one;
methyl (S)-3-((((8-(2,2′-dichloro-3′-(6-methoxy-5-((((5-oxopyrrolidin-2-yl)methyl)amino)methyl)pyridin-2-yl)-[1,1′-biphenyl]-3-yl)-4-oxo-4H-pyrido[1,2-a]pyrimidin-3-yl)methyl)amino)methyl)oxetane-3-carboxylate;
8-(2,2′-dichloro-3′-(6-methoxy-5-(((((S)-5-oxopyrrolidin-2-yl)methyl)amino)methyl)pyridin-2-yl)-[1,1′-biphenyl]-3-yl)-3-((((3,3-dimethyl-5-oxopyrrolidin-2-yl)methyl)amino)methyl)-4H-pyrido[1,2-a]pyrimidin-4-one;
8-(2,2′-dichloro-3′-(5-methoxy-6-(((((S)-5-oxopyrrolidin-2-yl)methyl)amino)methyl)pyridin-3-yl)-[1,1′-biphenyl]-3-yl)-3-(((((S)-5-oxopyrrolidin-2-yl)methyl)amino)methyl)-4H-pyrido[1,2-a]pyrimidin-4-one;
8-(2,2′-dichloro-3′-(6-((((1r,3r)-3-hydroxy-3-methylcyclobutyl)amino)methyl)-5-methoxypyridin-3-yl)-[1,1′-biphenyl]-3-yl)-3-((((1r,3r)-3-hydroxy-3-methylcyclobutyl)amino)methyl)-4H-pyrido[1,2-a]pyrimidin-4-one;
8-(2,2′-dichloro-3′-(6-(((2-hydroxyethyl)(methyl)amino)methyl)-5-methoxypyridin-3-yl)-[1,1′-biphenyl]-3-yl)-3-(((2-hydroxyethyl)(methyl)amino)methyl)-4H-pyrido[1,2-a]pyrimidin-4-one;
(S)-5-((((6-(2,2′-dichloro-3′-(1-methyl-2,3,4,5-tetrahydro-1H-benzo[e][1,4]diazepin-7-yl)-[1,1′-biphenyl]-3-yl)-2-methoxypyridin-3-yl)methyl)amino)methyl)pyrrolidin-2-on;
(S)-5-((((6-(2,2′-dichloro-3′-(1-methyl-2,3,4,5-tetrahydro-1H-benzo[e][1,4]diazepin-8-yl)-[1,1′-biphenyl]-3-yl)-2-methoxypyridin-3-yl)methyl)amino)methyl)pyrrolidin-2-one;
(S)-5-((((6-(2,2′-dichloro-3′-(1,2,3,4-tetrahydropyrazino[1,2-a]indol-8-yl)-[1,1′-biphenyl]-3-yl)-2-methoxypyridin-3-yl)methyl)amino)methyl)pyrrolidin-2-one;
(S)-5-((((3-chloro-5-(2,2′-dichloro-3′-(6-methoxy-5-(((((S)-5-oxopyrrolidin-2-yl)methyl)amino)methyl)pyridin-2-yl)-[1,1′-biphenyl]-3-yl)-1-methyl-1H-pyrrolo[3,2-b]pyridin-2-yl)methyl)amino)methyl)pyrrolidin-2-one
(S)-5-((((6-(2,2′-dichloro-3′-(8-chloro-6-((((R)-2-hydroxypropyl)amino)methyl)-5-methoxyquinolin-2-yl)-[1,1′-biphenyl]-3-yl)-2-methoxypyridin-3-yl)methyl)amino)methyl)pyrrolidin-2-one;
8-(2,2′-dichloro-3′-(6-methoxy-5-(((((S)-5-oxopyrrolidin-2-yl)methyl)amino)methyl)pyridin-2-yl)-[1,1′-biphenyl]-3-yl)-3-((((2-methyl-5-oxopyrrolidin-2-yl)methyl)amino)methyl)-4H-pyrido[1,2-a]pyrimidin-4-one;
(S)-5-((((6-(2,2′-dichloro-3′-(6-(((((S)-5-oxopyrrolidin-2-yl)methyl)amino)methyl)quinolin-2-yl)-[1,1′-biphenyl]-3-yl)-2-methoxypyridin-3-yl)methyl)amino)methyl)pyrrolidin-2-one;
(S)-5-((((3-(2,2′-dichloro-3′-(6-methoxy-5-(((((S)-5-oxopyrrolidin-2-yl)methyl)amino)methyl)pyridin-2-yl)-[1,1′-biphenyl]-3-yl)quinolin-7-yl)methyl)amino)methyl)pyrrolidin-2-one;
(S)-8-(2,2′-dichloro-3′-(6-methoxy-5-((((5-oxopyrrolidin-2-yl)methyl)amino)methyl)pyridin-2-yl)-[1,1′-biphenyl]-3-yl)-2-(((2-hydroxyethyl)amino)methyl)-3-methyl-4H-pyrido[1,2-a]pyrimidin-4-one;
8-(2,2′-dichloro-3′-(6-methoxy-5-(((((S)-5-oxopyrrolidin-2-yl)methyl)amino)methyl)pyridin-2-yl)-[1,1′-biphenyl]-3-yl)-3-methyl-2-(((((S)-5-oxopyrrolidin-2-yl)methyl)amino)methyl)-4H-pyrido[1,2-a]pyrimidin-4-one;
8-(2,2′-dichloro-3′-(6-methoxy-5-(((((S)-5-oxopyrrolidin-2-yl)methyl)amino)methyl)pyridin-2-yl)-[1,1′-biphenyl]-3-yl)-2-((((1r,3r)-3-hydroxy-3-methylcyclobutyl)amino)methyl)-3-methyl-4H-pyrido[1,2-a]pyrimidin-4-one;
(S)-10-(2,2′-dichloro-3′-(6-methoxy-5-((((5-oxopyrrolidin-2-yl)methyl)amino)methyl)pyridin-2-yl)-[1,1′-biphenyl]-3-yl)-1-methyl-2,3,4,5-tetrahydropyrido[1′,2′:1,2]pyrimido[4,5-e][1,4]diazepin-6(1H)-one;
(S)-5-((((6-(2,2′-dichloro-3′-(1-methyl-3-(((((S)-5-oxopyrrolidin-2-yl)methyl)amino)methyl)-1H-pyrrolo[3,2-b]pyridin-6-yl)-[1,1′-biphenyl]-3-yl)-2-methoxypyridin-3-yl)methyl)amino)methyl)pyrrolidin-2-one;
(S)-8-(2,2′-dichloro-3′-(6-methoxy-5-((((5-oxopyrrolidin-2-yl)methyl)amino)methyl)pyridin-2-yl)-[1,1′-biphenyl]-3-yl)-2-(((3-hydroxy-3-methylcyclobutyl)amino)methyl)-4H-pyrido[1,2-a]pyrimidin-4-one;
8-(2,2′-dichloro-3′-(6-methoxy-5-(((((S)-5-oxopyrrolidin-2-yl)methyl)amino)methyl)pyridin-2-yl)-[1,1′-biphenyl]-3-yl)-2-((((S)-2-hydroxypropyl)amino)methyl)-3-methyl-4H-pyrido[1,2-a]pyrimidin-4-one;
(S)-8-(2,2′-dichloro-3′-(4-chloro-6-methoxy-5-((((5-oxopyrrolidin-2-yl)methyl)amino)methyl)pyridin-2-yl)-[1,1′-biphenyl]-3-yl)-3-(((2-hydroxyethyl)amino)methyl)-4H-pyrido[1,2-a]pyrimidin-4-one;
(S)-5-((((6-(2,2′-dichloro-3′-(2-methoxy-3-(((((S)-5-oxopyrrolidin-2-yl)methyl)amino)methyl)pyrazolo[1,5-a]pyrimidin-6-yl)-[1,1′-biphenyl]-3-yl)-2-methoxypyridin-3-yl)methyl)amino)methyl)pyrrolidin-2-one;
(S)-5-((((6-(2,2′-dichloro-3′-(3-(((((S)-5-oxopyrrolidin-2-yl)methyl)amino)methyl)imidazo[1,2-a]pyridin-7-yl)-[1,1′-biphenyl]-3-yl)-2-methoxypyridin-3-yl)methyl)amino)methyl)pyrrolidin-2-one;
8-(2,2′-dichloro-3′-(3-(((((S)-5-oxopyrrolidin-2-yl)methyl)amino)methyl)pyrazolo[1,5-a]pyrimidin-6-yl)-[1,1′-biphenyl]-3-yl)-3-(((((S)-5-oxopyrrolidin-2-yl)methyl)amino)methyl)-4H-pyrido[1,2-a]pyrimidin-4-one;
(S)-5-((((3-chloro-7-(2,2′-dichloro-3′-(6-methoxy-5-(((((S)-5-oxopyrrolidin-2-yl)methyl)amino)methyl)pyridin-2-yl)-[1,1′-biphenyl]-3-yl)imidazo[1,2-a]pyridin-2-yl)methyl)amino)methyl)pyrrolidin-2-one;
2-(((3-chloro-7-(2,2′-dichloro-3′-(5-(((2-hydroxyethyl)amino)methyl)-6-methoxypyridin-2-yl)-[1,1′-biphenyl]-3-yl)imidazo[1,2-a]pyridin-2-yl)methyl)amino)ethan-1-ol;
methyl (S)-3-(((8-(2,2′-dichloro-3′-(6-methoxy-5-((((5-oxopyrrolidin-2-yl)methyl)amino)methyl)pyridin-2-yl)-[1,1′-biphenyl]-3-yl)-4-oxo-4H-pyrido[1,2-a]pyrimidin-3-yl)methyl)amino)-2,2-dimethylpropanoate;
(S)-5-((((3-chloro-5-(2,2′-dichloro-3′-(6-methoxy-5-(((((S)-5-oxopyrrolidin-2-yl)methyl)amino)methyl)pyridin-2-yl)-[1,1′-biphenyl]-3-yl)-1H-pyrrolo[3,2-b]pyridin-2-yl)methyl)amino)methyl)pyrrolidin-2-one;
(S)-5-((((3-chloro-6-(2,2′-dichloro-3′-(6-methoxy-5-(((((S)-5-oxopyrrolidin-2-yl)methyl)amino)methyl)pyridin-2-yl)-[1,1′-biphenyl]-3-yl)-1H-pyrrolo[2,3-b]pyridin-2-yl)methyl)amino)methyl)pyrrolidin-2-one;
2-(2,2′-dichloro-3′-(6-methoxy-5-(((((S)-5-oxopyrrolidin-2-yl)methyl)amino)methyl)pyridin-2-yl)-[1,1′-biphenyl]-3-yl)-5-(((((S)-5-oxopyrrolidin-2-yl)methyl)amino)methyl)isoindolin-1-one;
(S)-5-((((6-(2,2′-dichloro-3′-(1-methyl-3-(((((S)-5-oxopyrrolidin-2-yl)methyl)amino)methyl)-1H-pyrrolo[2,3-b]pyridin-6-yl)-[1,1′-biphenyl]-3-yl)-2-methoxypyridin-3-yl)methyl)amino)methyl)pyrrolidin-2-one;
(S)-5-((((6-(2,2′-dichloro-3′-(3-(((((S)-5-oxopyrrolidin-2-yl)methyl)amino)methyl)imidazo[1,2-a]pyridin-6-yl)-[1,1′-biphenyl]-3-yl)-2-methoxypyridin-3-yl)methyl)amino)methyl)pyrrolidin-2-one;
(5S)-5-((((6-(2,2′-dichloro-3′-(5-(((S)-2-hydroxypropyl)amino)-5,6,7,8-tetrahydroquinolin-2-yl)-[1,1′-biphenyl]-3-yl)-2-methoxypyridin-3-yl)methyl)amino)methyl)pyrrolidin-2-one;
(R)-2-(3-(6-(2,2′-dichloro-3′-(6-methoxy-5-((((5-oxopyrrolidin-2-yl)methyl)amino)methyl)pyridin-2-yl)-[1,1′-biphenyl]-3-yl)-3-oxo-[1,2,4]triazolo[4,3-a]pyridin-2(3H)-yl)propyl)isoindoline-1,3-dione;
(S)-6-(2,2′-dichloro-3′-(6-methoxy-5-((((5-oxopyrrolidin-2-yl)methyl)amino)methyl)pyridin-2-yl)-[1,1′-biphenyl]-3-yl)-2-(3-(diisobutylamino)propyl)-[1,2,4]triazolo[4,3-a]pyridin-3(2H)-one;
8-(2,2′-dichloro-3′-(4-chloro-6-methoxy-5-(((((S)-5-oxopyrrolidin-2-yl)methyl)amino)methyl)pyridin-2-yl)-[1,1′-biphenyl]-3-yl)-3-((((1r,3r)-3-hydroxy-3-methylcyclobutyl)amino)methyl)-4H-pyrido[1,2-a]pyrimidin-4-one;
(5S,5'S)-5,5′-(((2,2′-dichloro-[1,1′-biphenyl]-3,3′-diyl)bis(8-methoxy-3,4-dihydroisoquinoline-6,2(1H)-diyl))bis(methylene))bis(pyrrolidin-2-one);
(S)-6-(2,2′-dichloro-3′-(6-methoxy-5-((((5-oxopyrrolidin-2-yl)methyl)amino)methyl)pyridin-2-yl)-[1,1′-biphenyl]-3-yl)-2-(((2-hydroxyethyl)(methyl)amino)methyl)-3-methylpyrrolo[2,1-f][1,2,4]triazin-4(3H)-one;
(S)-10-(2,2′-dichloro-3′-(6-methoxy-5-((((5-oxopyrrolidin-2-yl)methyl)amino)methyl)pyridin-2-yl)-[1,1′-biphenyl]-3-yl)-2,3,4,5-tetrahydro-6H-pyrido[1′,2′:1,2]pyrimido[5,4-f][1,4]oxazepin-6-one;
(S)-5-((((6-(2,2′-dichloro-3′-(4-methoxy-3-(((((S)-5-oxopyrrolidin-2-yl)methyl)amino)methyl)pyrazolo[1,5-a]pyridin-6-yl)-[1,1′-biphenyl]-3-yl)-2-methoxypyridin-3-yl)methyl)amino)methyl)pyrrolidin-2-one;
(S)-5-((((8-chloro-2-(2,2′-dichloro-3′-(4-fluoro-6-methoxy-5-(((((S)-5-oxopyrrolidin-2-yl)methyl)amino)methyl)pyridin-2-yl)-[1,1′-biphenyl]-3-yl)-5-methoxyquinolin-6-yl)methyl)amino)methyl)pyrrolidin-2-one;
8-(2,2′-dichloro-3′-(6-methoxy-5-(((((S)-5-oxopyrrolidin-2-yl)methyl)amino)methyl)pyridin-2-yl)-[1,1′-biphenyl]-3-yl)-2-ethyl-3-(((((S)-5-oxopyrrolidin-2-yl)methyl)amino)methyl)-4H-pyrido[1,2-a]pyrimidin-4-one;
6-(2,2′-dichloro-3′-(6-methoxy-5-(((((S)-5-oxopyrrolidin-2-yl)methyl)amino)methyl)pyridin-2-yl)-[1,1′-biphenyl]-3-yl)-8-fluoro-2-(2-((((S)-5-oxopyrrolidin-2-yl)methyl)amino)ethyl)-3,4-dihydroisoquinolin-1(2H)-one;
(S)-5-((((2-(2,2′-dichloro-3′-(6-methoxy-5-(((((S)-5-oxopyrrolidin-2-yl)methyl)amino)methyl)pyridin-2-yl)-[1,1′-biphenyl]-3-yl)-7-methyl-7H-pyrrolo[2,3-d]pyrimidin-5-yl)methyl)amino)methyl)pyrrolidin-2-one;
(S)-5-((((6-(2,2′-dichloro-3′-(1-methyl-3-(((((S)-5-oxopyrrolidin-2-yl)methyl)amino)methyl)-1H-pyrrolo[3,2-c]pyridin-6-yl)-[1,1′-biphenyl]-3-yl)-2-methoxypyridin-3-yl)methyl)amino)methyl)pyrrolidin-2-one;
(S)-7-(2,2′-dichloro-3′-(6-methoxy-5-((((5-oxopyrrolidin-2-yl)methyl)amino)methyl)pyridin-2-yl)-[1,1′-biphenyl]-3-yl)-1,3,4,5-tetrahydro-2H-pyrido[2,3-e][1,4]diazepin-2-one;
8-(2,2′-dichloro-3′-(4-fluoro-6-methoxy-5-(((((S)-5-oxopyrrolidin-2-yl)methyl)amino)methyl)pyridin-2-yl)-[1,1′-biphenyl]-3-yl)-2-methyl-3-(((((S)-5-oxopyrrolidin-2-yl)methyl)amino)methyl)-4H-pyrido[1,2-a]pyrimidin-4-one;
(S)-6-(2,2′-dichloro-3′-(6-methoxy-5-((((5-oxopyrrolidin-2-yl)methyl)amino)methyl)pyridin-2-yl)-[1,1′-biphenyl]-3-yl)-2-(((2-hydroxy-2-methylpropyl)amino)methyl)-3-methylpyrrolo[2,1-f][1,2,4]triazin-4(3H)-one;
(S)-6-(2,2′-dichloro-3′-(6-methoxy-5-((((5-oxopyrrolidin-2-yl)methyl)amino)methyl)pyridin-2-yl)-[1,1′-biphenyl]-3-yl)-3-methyl-2-(((3,3,3-trifluoropropyl)amino)methyl)pyrrolo[2,1-f][1,2,4]triazin-4(3H)-one;
8-(2,2′-dichloro-3′-(6-methoxy-5-(((((S)-5-oxopyrrolidin-2-yl)methyl)amino)methyl)pyridin-2-yl)-[1,1′-biphenyl]-3-yl)-3-((((2-(hydroxymethyl)cyclopropyl)methyl)amino)methyl)-4H-pyrido[1,2-a]pyrimidin-4-one;
(S)-8-(2,2′-dichloro-3′-(1-methyl-2,3,4,5-tetrahydro-1H-benzo[e][1,4]diazepin-8-yl)-[1,1′-biphenyl]-3-yl)-3-((((5-oxopyrrolidin-2-yl)methyl)amino)methyl)-4H-pyrido[1,2-a]pyrimidin-4-one;
(S)-5-((((6-(2,2′-dichloro-3′-(3-((((S)-2-hydroxypropyl)amino)methyl)-1-methyl-1H-pyrrolo[2,3-b]pyridin-6-yl)-[1,1′-biphenyl]-3-yl)-2-methoxypyridin-3-yl)methyl)amino)methyl)pyrrolidin-2-one;
(S)-5-((((6-(2,2′-dichloro-3′-(3-(((2-hydroxyethyl)(methyl)amino)methyl)-1-methyl-1H-pyrrolo[2,3-b]pyridin-6-yl)-[1,1′-biphenyl]-3-yl)-2-methoxypyridin-3-yl)methyl)amino)methyl)pyrrolidin-2-one;
(S)-5-((((6-(2,2′-dichloro-3′-(1-methyl-3-(((((S)-oxetan-2-yl)methyl)amino)methyl)-1H-pyrrolo[2,3-b]pyridin-6-yl)-[1,1′-biphenyl]-3-yl)-2-methoxypyridin-3-yl)methyl)amino)methyl)pyrrolidin-2-one;
(S)-5-((((6-(2,2′-dichloro-3′-(1-methyl-3-(((2-(2-oxopyrrolidin-1-yl)ethyl)amino)methyl)-1H-pyrrolo[2,3-b]pyridin-6-yl)-[1,1′-biphenyl]-3-yl)-2-methoxypyridin-3-yl)methyl)amino)methyl)pyrrolidin-2-one;
(S)-5-((((6-(3′-(3-(((1-acetylpiperidin-4-yl)amino)methyl)-1-methyl-1H-pyrrolo[2,3-15 b]pyridin-6-yl)-2,2′-dichloro-[1,1′-biphenyl]-3-yl)-2-methoxypyridin-3-yl)methyl)amino)methyl)pyrrolidin-2-one;
8-(2,2′-dichloro-3′-(1-methyl-3-(((((S)-5-oxopyrrolidin-2-yl)methyl)amino)methyl)-1H-pyrrolo[2,3-b]pyridin-6-yl)-[1,1′-biphenyl]-3-yl)-3-(((((S)-5-oxopyrrolidin-2-yl)methyl)amino)methyl)-4H-pyrido[1,2-a]pyrimidin-4-one;
(5S,5'S)-5,5′-(((((2,2′-dichloro-[1,1′-biphenyl]-3,3′-diyl)bis(1-methyl-1H-pyrrolo[2,3-b]pyridine-6,3-diyl))bis(methylene))bis(azanediyl))bis(methylene))bis(pyrrolidin-2-one);
(S)-5-((((6-(2,2′-dichloro-3′-(2-((S)-2-hydroxypropyl)-8-methoxy-1,2,3,4-tetrahydroisoquinolin-6-yl)-[1,1′-biphenyl]-3-yl)-2-methoxypyridin-3-yl)methyl)amino)methyl)pyrrolidin-2-one;
(S)-5-((((6-(2,2′-dichloro-3′-(2-((R)-2-hydroxypropyl)-8-methoxy-1,2,3,4-tetrahydroisoquinolin-6-yl)-[1,1′-biphenyl]-3-yl)-2-methoxypyridin-3-yl)methyl)amino)methyl)pyrrolidin-2-one;
(S)-5-((((6-(2,2′-dichloro-3′-(2-methyl-3-(((((S)-5-oxopyrrolidin-2-yl)methyl)amino)methyl)pyrazolo[1,5-a]pyrimidin-6-yl)-[1,1′-biphenyl]-3-yl)-2-methoxypyridin-3-yl)methyl)amino)methyl)pyrrolidin-2-one;
7-(2,2′-dichloro-3′-(6-methoxy-5-(((((S)-5-oxopyrrolidin-2-yl)methyl)amino)methyl)pyridin-2-yl)-[1,1′-biphenyl]-3-yl)-2-(((((S)-5-oxopyrrolidin-2-yl)methyl)amino)methyl)quinazolin-4(1H)-one;
(S)-5-((((6-(2,2′-dichloro-3′-(2-(((((S)-5-oxopyrrolidin-2-yl)methyl)amino)methyl)quinazolin-6-yl)-[1,1′-biphenyl]-3-yl)-2-methoxypyridin-3-yl)methyl)amino)methyl)pyrrolidin-2-one;
tert-butyl (S)-((6-(2,2′-dichloro-3′-(2-(hydroxymethyl)-3-methyl-4-oxo-3,4-dihydropyrrolo[2,1-f][1,2,4]triazin-6-yl)-[1,1′-biphenyl]-3-yl)-2-methoxypyridin-3-yl)methyl)((5-oxopyrrolidin-2-yl)methyl)carbamate;
(S)-5-((((6-(2,2′-dichloro-3′-(5-methoxy-2-(((S)-5-oxopyrrolidin-2-yl)methyl)-1,2,3,4-tetrahydroisoquinolin-7-yl)-[1,1′-biphenyl]-3-yl)-2-methoxypyridin-3-yl)methyl)amino)methyl)pyrrolidin-2-one;
(S)-5-((6-(2,2′-dichloro-3′-(6-methoxy-5-(((((S)-5-oxopyrrolidin-2-yl)methyl)amino)methyl)pyridin-2-yl)-[1,1′-biphenyl]-3-yl)-8-fluoro-3,4-dihydroisoquinolin-2(1H)-yl)methyl)pyrrolidin-2-one;
(S)-5-((((6-(2,2′-dichloro-3′-(6-(((S)-5-oxopyrrolidin-2-yl)methyl)-5,6,7,8-tetrahydro-1,6-naphthyridin-2-yl)-[1,1′-biphenyl]-3-yl)-2-methoxypyridin-3-yl)methyl)amino)methyl)pyrrolidin-2-one;
(S)-5-((8-chloro-7-(2,2′-dichloro-3′-(6-methoxy-5-(((((S)-5-oxopyrrolidin-2-yl)methyl)amino)methyl)pyridin-2-yl)-[1,1′-biphenyl]-3-yl)-3,4-dihydroisoquinolin-2(1H)-yl)methyl)pyrrolidin-2-one;
(S)-5-((7-(2,2′-dichloro-3′-(6-methoxy-5-(((((S)-5-oxopyrrolidin-2-yl)methyl)amino)methyl)pyridin-2-yl)-[1,1′-biphenyl]-3-yl)-8-fluoro-3,4-dihydroisoquinolin-2(1H)-yl)methyl)pyrrolidin-2-one;
8-(2,2′-dichloro-3′-(3-chloro-2-(((((S)-5-oxopyrrolidin-2-yl)methyl)amino)methyl)imidazo[1,2-a]pyridin-7-yl)-[1,1′-biphenyl]-3-yl)-3-(((((S)-5-oxopyrrolidin-2-yl)methyl)amino)methyl)-4H-pyrido[1,2-a]pyrimidin-4-one;
(5S,5'S)-5,5′-(((((2,2′-dichloro-[1,1′-biphenyl]-3,3′-diyl)bis(3-chloroimidazo[1,2-a]pyridine-7,2-diyl))bis(methylene))bis(azanediyl))bis(methylene))bis(pyrrolidin-2-one);
8-(2,2′-dichloro-3′-(6-chloro-4-methoxy-5-(((((S)-5-oxopyrrolidin-2-yl)methyl)amino)methyl)pyridin-2-yl)-[1,1′-biphenyl]-3-yl)-3-(((((S)-5-oxopyrrolidin-2-yl)methyl)amino)methyl)-4H-pyrido[1,2-a]pyrimidin-4-one;
(5S)-5-(((3-(2,2′-dichloro-3′-(6-methoxy-5-(((((S)-5-oxopyrrolidin-2-yl)methyl)amino)methyl)pyridin-2-yl)-[1,1′-biphenyl]-3-yl)-5,6,7,8-tetrahydroquinolin-8-yl)amino)methyl)pyrrolidin-2-one;
6-(2,2′-dichloro-3′-(6-methoxy-5-(((((S)-5-oxopyrrolidin-2-yl)methyl)amino)methyl)pyridin-2-yl)-[1,1′-biphenyl]-3-yl)-2-(((((S)-5-oxopyrrolidin-2-yl)methyl)amino)methyl)quinazolin-4(1H)-one;
(S)-5-((((6-(2,2′-dichloro-3′-(3-(((((S)-5-oxopyrrolidin-2-yl)methyl)amino)methyl)-1H-pyrazolo[3,4-b]pyridin-5-yl)-[1,1′-biphenyl]-3-yl)-2-methoxypyridin-3-yl)methyl)amino)methyl)pyrrolidin-2-one;
(5S)-5-((((6-(2,2′-dichloro-3′-(5-((((S)-5-oxopyrrolidin-2-yl)methyl)amino)-5,6,7,8-tetrahydroquinolin-2-yl)-[1,1′-biphenyl]-3-yl)-2-methoxypyridin-3-yl)methyl)amino)methyl)pyrrolidin-2-one;
6-(2,2′-dichloro-3′-(6-methoxy-5-(((((S)-5-oxopyrrolidin-2-yl)methyl)amino)methyl)pyridin-2-yl)-[1,1′-biphenyl]-3-yl)-2-((((S)-2-hydroxypropyl)(methyl)amino)methyl)-3-methylpyrrolo[2,1-f][1,2,4]triazin-4(3H)-one;
(S)-6-(2,2′-dichloro-3′-(6-methoxy-5-((((5-oxopyrrolidin-2-yl)methyl)amino)methyl)pyridin-2-yl)-[1,1′-biphenyl]-3-yl)-2-(((3-fluoropropyl)amino)methyl)-3-methylpyrrolo[2,1-f][1,2,4]triazin-4(3H)-one;
(S)—N-(2-aminoethyl)-6-(2,2′-dichloro-3′-(6-methoxy-5-((((5-oxopyrrolidin-2-yl)methyl)amino)methyl)pyridin-2-yl)-[1,1′-biphenyl]-3-yl)imidazo[1,2-b]pyridazine-2-carboxamide;
N—((S)-2-aminopropyl)-6-(2,2′-dichloro-3′-(6-methoxy-5-(((((S)-5-oxopyrrolidin-2-yl)methyl)amino)methyl)pyridin-2-yl)-[1,1′-biphenyl]-3-yl)imidazo[1,2-b]pyridazine-2-carboxamide;
(S)—N-(2-(1H-imidazol-4-yl)ethyl)-6-(2,2′-dichloro-3′-(6-methoxy-5-((((5-oxopyrrolidin-2-yl)methyl)amino)methyl)pyridin-2-yl)-[1,1′-biphenyl]-3-yl)imidazo[1,2-b]pyridazine-2-carboxamid;
8-(2,2′-dichloro-3′-(2-(((((S)-5-oxopyrrolidin-2-yl)methyl)amino)methyl)thiazol-5-yl)-[1,1′-biphenyl]-3-yl)-3-(((((S)-5-oxopyrrolidin-2-yl)methyl)amino)methyl)-4H-pyrido[1,2-a]pyrimidin-4-one;
8-(2,2′-dichloro-3′-(2-(((((S)-5-oxopyrrolidin-2-yl)methyl)amino)methyl)thiazol-4-yl)-[1,1′-biphenyl]-3-yl)-3-(((((S)-5-oxopyrrolidin-2-yl)methyl)amino)methyl)-4H-pyrido[1,2-a]pyrimidin-4-one;
(S)-5-((((6-(2,2′-dichloro-3′-(6-(((S)-5-oxopyrrolidin-2-yl)methyl)-5,6,7,8-tetrahydropyrido[4,3-d]pyrimidin-2-yl)-[1,1′-biphenyl]-3-yl)-2-methoxypyridin-3-yl)methyl)amino)methyl)pyrrolidin-2-one;
(S)-5-((((6-(2,2′-dichloro-3′-(1-methyl-3-(((((S)-tetrahydrofuran-2-yl)methyl)amino)methyl)-1H-pyrrolo[2,3-b]pyridin-6-yl)-[1,1′-biphenyl]-3-yl)-2-methoxypyridin-3-yl)methyl)amino)methyl)pyrrolidin-2-one;
(S)-5-((((6-(2,2′-dichloro-3′-(3-((((S)-2-methoxypropyl)amino)methyl)-1-methyl-1H-pyrrolo[2,3-b]pyridin-6-yl)-[1,1′-biphenyl]-3-yl)-2-methoxypyridin-3-yl)methyl)amino)methyl)pyrrolidin-2-one;
(S)-2-(((2-oxaspiro[3.3]heptan-6-yl)amino)methyl)-6-(2,2′-dichloro-3′-(6-methoxy-5-((((5-oxopyrrolidin-2-yl)methyl)amino)methyl)pyridin-2-yl)-[1,1′-biphenyl]-3-yl)-3-methylpyrrolo[2,1-f][1,2,4]triazin-4(3H)-one;
(S)-2-((2-oxa-6-azaspiro[3.3]heptan-6-yl)methyl)-6-(2,2′-dichloro-3′-(6-methoxy-5-((((5-oxopyrrolidin-2-yl)methyl)amino)methyl)pyridin-2-yl)-[1,1′-biphenyl]-3-yl)-3-methylpyrrolo[2,1-f][1,2,4]triazin-4(3H)-one;
(S)-6-(2,2′-dichloro-3′-(6-methoxy-5-((((5-oxopyrrolidin-2-yl)methyl)amino)methyl)pyridin-2-yl)-[1,1′-biphenyl]-3-yl)-3-methyl-2-((6-oxo-2,5-diazaspiro[3.4]octan-2-yl)methyl)pyrrolo[2,1-f][1,2,4]triazin-4(3H)-one;
(S)-6-(2,2′-dichloro-3′-(6-methoxy-5-((((5-oxopyrrolidin-2-yl)methyl)amino)methyl)pyridin-2-yl)-[1,1′-biphenyl]-3-yl)-3-methyl-2-(((6-oxo-5-azaspiro[3.4]octan-2-yl)amino)methyl)pyrrolo[2,1-f][1,2,4]triazin-4(3H)-one;
6-(2,2′-dichloro-3′-(6-methoxy-5-(((((S)-5-oxopyrrolidin-2-yl)methyl)amino)methyl)pyridin-2-yl)-[1,1′-biphenyl]-3-yl)-2-((((4,4-difluoro-5-oxopyrrolidin-2-yl)methyl)amino)methyl)-3-methylpyrrolo[2,1-f][1,2,4]triazin-4(3H)-one;
6-(2,2′-dichloro-3′-(6-methoxy-5-(((((S)-5-oxopyrrolidin-2-yl)methyl)amino)methyl)pyridin-2-yl)-[1,1′-biphenyl]-3-yl)-3-methyl-2-(((2-(5-oxopyrrolidin-2-yl)ethyl)amino)methyl)pyrrolo[2,1-f][1,2,4]triazin-4(3H)-one;
(S)-5-((((6-(2,2′-dichloro-3′-(1-methyl-3-(((((S)-5-oxopyrrolidin-2-yl)methyl)amino)methyl)-1H-indol-6-yl)-[1,1′-biphenyl]-3-yl)-2-methoxypyridin-3-yl)methyl)amino)methyl)pyrrolidin-2-one;
8-(2,2′-dichloro-3′-(1-methyl-3-(((((S)-5-oxopyrrolidin-2-yl)methyl)amino)methyl)-1H-indol-6-yl)-[1,1′-biphenyl]-3-yl)-3-(((((S)-5-oxopyrrolidin-2-yl)methyl)amino)methyl)-4H-pyrido[1,2-a]pyrimidin-4-one;
6-(2,2′-dichloro-3′-(6-methoxy-5-(((((S)-5-oxopyrrolidin-2-yl)methyl)amino)methyl)pyridin-2-yl)-[1,1′-biphenyl]-3-yl)-2-(2-((((S)-5-oxopyrrolidin-2-yl)methyl)amino)ethyl)-2,3-dihydro-1H-pyrrolo[3,4-c]pyridin-1-one;
(S)-5-((((6-(2,2′-dichloro-3′-(2-(2-hydroxyethyl)-8-methoxy-1,2,3,4-tetrahydroisoquinolin-6-yl)-[1,1′-biphenyl]-3-yl)-2-methoxypyridin-3-yl)methyl)amino)methyl)pyrrolidin-2-one;
(S)-5-((((6-(2,2′-dichloro-3′-((R)-5-((((S)-5-oxopyrrolidin-2-yl)methyl)amino)-5,6,7,8-tetrahydroquinolin-2-yl)-[1,1′-biphenyl]-3-yl)-2-methoxypyridin-3-yl)methyl)amino)methyl)pyrrolidin-2-one;
(S)-2-(((1-acetylpiperidin-4-yl)amino)methyl)-6-(2,2′-dichloro-3′-(6-methoxy-5-((((5-oxopyrrolidin-2-yl)methyl)amino)methyl)pyridin-2-yl)-[1,1′-biphenyl]-3-yl)-3-methylpyrrolo[2,1-f][1,2,4]triazin-4(3H)-one;
(S)-6-(2,2′-dichloro-3′-(6-methoxy-5-((((5-oxopyrrolidin-2-yl)methyl)amino)methyl)pyridin-2-yl)-[1,1′-biphenyl]-3-yl)-3-methyl-2-(((2-(2-oxopyrrolidin-1-yl)ethyl)amino)methyl)pyrrolo[2,1-f][1,2,4]triazin-4(3H)-one;
(S)-5-((((6-(2,2′-dichloro-3′-(2-(((((S)-5-oxopyrrolidin-2-yl)methyl)amino)methyl)furo[2,3-b]pyridin-5-yl)-[1,1′-biphenyl]-3-yl)-2-methoxypyridin-3-yl)methyl)amino)methyl)pyrrolidin-2-one;
(S)-2-((6-acetyl-2,6-diazaspiro[3.3]heptan-2-yl)methyl)-6-(2,2′-dichloro-3′-(6-methoxy-5-((((5-oxopyrrolidin-2-yl)methyl)amino)methyl)pyridin-2-yl)-[1,1′-biphenyl]-3-yl)-3-methylpyrrolo[2,1-f][1,2,4]triazin-4(3H)-one;
(S)-6-(2,2′-dichloro-3′-(6-methoxy-5-((((5-oxopyrrolidin-2-yl)methyl)amino)methyl)pyridin-2-yl)-[1,1′-biphenyl]-3-yl)-2-((3-(2-hydroxypropan-2-yl)azetidin-1-yl)methyl)-3-methylpyrrolo[2,1-f][1,2,4]triazin-4(3H)-one;
(S)-6-(2,2′-dichloro-3′-(6-methoxy-5-((((5-oxopyrrolidin-2-yl)methyl)amino)methyl)pyridin-2-yl)-[1,1′-biphenyl]-3-yl)-2-(((2-fluoro-2-methylpropyl)amino)methyl)-3-methylpyrrolo[2,1-f][1,2,4]triazin-4(3H)-one;
(S)-6-(2,2′-dichloro-3′-(6-methoxy-5-((((5-oxopyrrolidin-2-yl)methyl)amino)methyl)pyridin-2-yl)-[1,1′-biphenyl]-3-yl)-2-((((1-fluorocyclopropyl)methyl)amino)methyl)-3-methylpyrrolo[2,1-f][1,2,4]triazin-4(3H)-one;
6-(2,2′-dichloro-3′-(4-chloro-6-methoxy-5-(((((S)-5-oxopyrrolidin-2-yl)methyl)amino)methyl)pyridin-2-yl)-[1,1′-biphenyl]-3-yl)-2-((((1r,3r)-3-hydroxy-3-methylcyclobutyl)amino)methyl)-3-methylpyrrolo[2,1-f][1,2,4]triazin-4(3H)-one;
(S)-6-(2,2′-dichloro-3′-(6-methoxy-5-((((5-oxopyrrolidin-2-yl)methyl)amino)methyl)pyridin-2-yl)-[1,1′-biphenyl]-3-yl)-2-(((3,3-difluoropropyl)amino)methyl)-3-methylpyrrolo[2,1-f][1,2,4]triazin-4(3H)-one;
(S)-6-(2,2′-dichloro-3′-(6-methoxy-5-((((5-oxopyrrolidin-2-yl)methyl)amino)methyl)pyridin-2-yl)-[1,1′-biphenyl]-3-yl)-3-methyl-2-(((3,3,3-trifluoro-2,2-dimethylpropyl)amino)methyl)pyrrolo[2,1-f][1,2,4]triazin-4(3H)-one;
6-(2,2′-dichloro-3′-(6-methoxy-5-(((((S)-5-oxopyrrolidin-2-yl)methyl)amino)methyl)pyridin-2-yl)-[1,1′-biphenyl]-3-yl)-2-(2-((((S)-5-oxopyrrolidin-2-yl)methyl)amino)ethyl)-1,2-dihydro-3H-pyrrolo[3,4-c]pyridin-3-one;
(S)-6-(2,2′-dichloro-3′-(6-methoxy-5-((((5-oxopyrrolidin-2-yl)methyl)amino)methyl)pyridin-2-yl)-[1,1′-biphenyl]-3-yl)-2-((6-hydroxy-2-azaspiro[3.3]heptan-2-yl)methyl)-3-methylpyrrolo[2,1-f][1,2,4]triazin-4(3H)-one;
(S)-6-(2,2′-dichloro-3′-(6-methoxy-5-((((5-oxopyrrolidin-2-yl)methyl)amino)methyl)pyridin-2-yl)-[1,1′-biphenyl]-3-yl)-2-((3-hydroxy-3-methylazetidin-1-yl)methyl)-3-methylpyrrolo[2,1-f][1,2,4]triazin-4(3H)-one;
(S)-1-((6-(2,2′-dichloro-3′-(6-methoxy-5-((((5-oxopyrrolidin-2-yl)methyl)amino)methyl)pyridin-2-yl)-[1,1′-biphenyl]-3-yl)-3-methyl-4-oxo-3,4-dihydropyrrolo[2,1-f][1,2,4]triazin-2-yl)methyl)-3-methylazetidine-3-carbonitrile;
(S)-5-((((6-(2,2′-dichloro-3′-(4-methoxy-6-(((S)-5-oxopyrrolidin-2-yl)methyl)-5,6,7,8-tetrahydropyrido[4,3-d]pyrimidin-2-yl)-[1,1′-biphenyl]-3-yl)-2-methoxypyridin-3-yl)methyl)amino)methyl)pyrrolidin-2-one;
6-(2,2′-dichloro-3′-(6-methoxy-5-(((((S)-5-oxopyrrolidin-2-yl)methyl)amino)methyl)pyridin-2-yl)-[1,1′-biphenyl]-3-yl)-3-methyl-2-(((((S)-oxetan-2-yl)methyl)amino)methyl)pyrrolo[2,1-f][1,2,4]triazin-4(3H)-one;
(S)-6-(2,2′-dichloro-3′-(6-methoxy-5-((((5-oxopyrrolidin-2-yl)methyl)amino)methyl)pyridin-2-yl)-[1,1′-biphenyl]-3-yl)-2-(((3-hydroxybicyclo[1.1.1]pentan-1-yl)amino)methyl)-3-methylpyrrolo[2,1-f][1,2,4]triazin-4(3H)-one;
6-(2,2′-dichloro-3′-(6-methoxy-5-(((((S)-5-oxopyrrolidin-2-yl)methyl)amino)methyl)pyridin-2-yl)-[1,1′-biphenyl]-3-yl)-3-methyl-2-(((((S)-tetrahydrofuran-2-yl)methyl)amino)methyl)pyrrolo[2,1-f][1,2,4]triazin-4(3H)-one;
6-(2,2′-dichloro-3′-(6-methoxy-5-(((((S)-5-oxopyrrolidin-2-yl)methyl)amino)methyl)pyridin-2-yl)-[1,1′-biphenyl]-3-yl)-2-((((S)-2-methoxypropyl)amino)methyl)-3-methylpyrrolo[2,1-f][1,2,4]triazin-4(3H)-one;
6-(2,2′-dichloro-3′-(6-methoxy-5-(((((S)-5-oxopyrrolidin-2-yl)methyl)amino)methyl)pyridin-2-yl)-[1,1′-biphenyl]-3-yl)-3-methyl-2-(((((S)-5-oxopyrrolidin-3-yl)methyl)amino)methyl)pyrrolo[2,1-f][1,2,4]triazin-4(3H)-one;
(S)-6-(2,2′-dichloro-3′-(6-methoxy-5-((((5-oxopyrrolidin-2-yl)methyl)amino)methyl)pyridin-2-yl)-[1,1′-biphenyl]-3-yl)-2-((isopropylamino)methyl)-3-methylpyrrolo[2,1-f][1,2,4]triazin-4(3H)-one;
(S)-6-(2,2′-dichloro-3′-(4-chloro-6-methoxy-5-((((5-oxopyrrolidin-2-yl)methyl)amino)methyl)pyridin-2-yl)-[1,1′-biphenyl]-3-yl)-2-(((2-hydroxy-2-methylpropyl)amino)methyl)-3-methylpyrrolo[2,1-f][1,2,4]triazin-4(3H)-one;
(S)-2-((tert-butylamino)methyl)-6-(2,2′-dichloro-3′-(6-methoxy-5-((((5-oxopyrrolidin-2-yl)methyl)amino)methyl)pyridin-2-yl)-[1,1′-biphenyl]-3-yl)-3-methylpyrrolo[2,1-f][1,2,4]triazin-4(3H)-one;
(S)-6-(2,2′-dichloro-3′-(4-fluoro-6-methoxy-5-((((5-oxopyrrolidin-2-yl)methyl)amino)methyl)pyridin-2-yl)-[1,1′-biphenyl]-3-yl)-2-(((3-hydroxy-3-methylcyclobutyl)amino)methyl)-3-methylpyrrolo[2,1-f][1,2,4]triazin-4(3H)-one;
(S)-4-(((6-(2,2′-dichloro-3′-(6-methoxy-5-(((((S)-5-oxopyrrolidin-2-yl)methyl)amino)methyl)pyridin-2-yl)-[1,1′-biphenyl]-3-yl)-3-methyl-4-oxo-3,4-dihydropyrrolo[2,1-f][1,2,4]triazin-2-yl)methyl)amino)-3-hydroxy-N,N-dimethylbutanamide;
methyl (S)-((6-(2,2′-dichloro-3′-(6-methoxy-5-((((5-oxopyrrolidin-2-yl)methyl)amino)methyl)pyridin-2-yl)-[1,1′-biphenyl]-3-yl)-3-methyl-4-oxo-3,4-dihydropyrrolo[2,1-f][1,2,4]triazin-2-yl)methyl)glycinate;
2-(2,2′-dichloro-3′-(6-methoxy-5-(((((S)-5-oxopyrrolidin-2-yl)methyl)amino)methyl)pyridin-2-yl)-[1,1′-biphenyl]-3-yl)-6-(((((S)-5-oxopyrrolidin-2-yl)methyl)amino)methyl)isoindolin-1-one;
(S)-5-((((6-(2,2′-dichloro-3′-(2-(2-(1-hydroxycyclobutyl)ethyl)-8-methoxy-1,2,3,4-tetrahydroisoquinolin-6-yl)-[1,1′-biphenyl]-3-yl)-2-methoxypyridin-3-yl)methyl)amino)methyl)pyrrolidin-2-one;
(S)-5-((((6-(2,2′-dichloro-3′-(2-(3-hydroxy-3-methylbutyl)-8-methoxy-1,2,3,4-tetrahydroisoquinolin-6-yl)-[1,1′-biphenyl]-3-yl)-2-methoxypyridin-3-yl)methyl)amino)methyl)pyrrolidin-2-one;
6-(2,2′-dichloro-3′-(6-methoxy-5-(((((S)-5-oxopyrrolidin-2-yl)methyl)amino)methyl)pyridin-2-yl)-[1,1′-biphenyl]-3-yl)-2-((((lr,4r)-4-hydroxycyclohexyl)amino)methyl)-3-methylpyrrolo[2,1-f][1,2,4]triazin-4(3H)-one;
(S)-((6-(2,2′-dichloro-3′-(6-methoxy-5-((((5-oxopyrrolidin-2-yl)methyl)amino)methyl)pyridin-2-yl)-[1,1′-biphenyl]-3-yl)-3-methyl-4-oxo-3,4-dihydropyrrolo[2,1-f][1,2,4]triazin-2-yl)methyl)glycine;
(5S)-5-((((6-(2,2′-dichloro-3′-(2-(2-hydroxy-3-methylbutyl)-8-methoxy-1,2,3,4-tetrahydroisoquinolin-6-yl)-[1,1′-biphenyl]-3-yl)-2-methoxypyridin-3-yl)methyl)amino)methyl)pyrrolidin-2-one;
methyl (S)-4-(((6-(2,2′-dichloro-3′-(6-methoxy-5-(((((S)-5-oxopyrrolidin-2-yl)methyl)amino)methyl)pyridin-2-yl)-[1,1′-biphenyl]-3-yl)-3-methyl-4-oxo-3,4-dihydropyrrolo[2,1-f][1,2,4]triazin-2-yl)methyl)amino)-3-hydroxybutanoate;
(S)-6-(2,2′-dichloro-3′-(4-chloro-6-methoxy-5-((((5-oxopyrrolidin-2-yl)methyl)amino)methyl)pyridin-2-yl)-[1,1′-biphenyl]-3-yl)-2-(((3-fluoropropyl)amino)methyl)-3-methylpyrrolo[2,1-f][1,2,4]triazin-4(3H)-one;
(S)-6-(2,2′-dichloro-3′-(4-chloro-6-methoxy-5-((((5-oxopyrrolidin-2-yl)methyl)amino)methyl)pyridin-2-yl)-[1,1′-biphenyl]-3-yl)-2-(((2-hydroxyethyl)amino)methyl)-3-methylpyrrolo[2,1-f][1,2,4]triazin-4(3H)-one;
(S)-6-(2,2′-dichloro-3′-(4-chloro-6-methoxy-5-((((5-oxopyrrolidin-2-yl)methyl)amino)methyl)pyridin-2-yl)-[1,1′-biphenyl]-3-yl)-2-((3-hydroxy-3-methylazetidin-1-yl)methyl)-3-methylpyrrolo[2,1-f][1,2,4]triazin-4(3H)-one;
(S)-4-(((6-(2,2′-dichloro-3′-(6-methoxy-5-(((((S)-5-oxopyrrolidin-2-yl)methyl)amino)methyl)pyridin-2-yl)-[1,1′-biphenyl]-3-yl)-3-methyl-4-oxo-3,4-dihydropyrrolo[2,1-f][1,2,4]triazin-2-yl)methyl)amino)-3-hydroxybutanoic acid;
(S)-1-((6-(2,2′-dichloro-3′-(6-methoxy-5-(((((S)-5-oxopyrrolidin-2-yl)methyl)amino)methyl)pyridin-2-yl)-[1,1′-biphenyl]-3-yl)-2-methoxypyridin-3-yl)methyl)piperidine-2-carboxylic acid;
methyl (S)-3-(((6-(2,2′-dichloro-3′-(6-methoxy-5-((((5-oxopyrrolidin-2-yl)methyl)amino)methyl)pyridin-2-yl)-[1,1′-biphenyl]-3-yl)-3-methyl-4-oxo-3,4-dihydropyrrolo[2,1-f][1,2,4]triazin-2-yl)methyl)amino)-2,2-dimethylpropanoate;
methyl (S)-1-((6-(2,2′-dichloro-3′-(6-methoxy-5-((((5-oxopyrrolidin-2-yl)methyl)amino)methyl)pyridin-2-yl)-[1,1′-biphenyl]-3-yl)-3-methyl-4-oxo-3,4-dihydropyrrolo[2,1-f][1,2,4]triazin-2-yl)methyl)azetidine-3-carboxylate;
(S)-5-((((6-(2,2′-dichloro-3′-(8-methoxy-2-(2-(piperidin-4-yl)ethyl)-1,2,3,4-tetrahydroisoquinolin-6-yl)-[1,1′-biphenyl]-3-yl)-2-methoxypyridin-3-yl)methyl)amino)methyl)pyrrolidin-2-one;
(S)-5-((((6-(3′-(2-((1-acetylpiperidin-4-yl)methyl)-8-methoxy-1,2,3,4-tetrahydroisoquinolin-6-yl)-2,2′-dichloro-[1,1′-biphenyl]-3-yl)-2-methoxypyridin-3-yl)methyl)amino)methyl)pyrrolidin-2-one;
(S)-6-(2,2′-dichloro-3′-(4-fluoro-6-methoxy-5-((((5-oxopyrrolidin-2-yl)methyl)amino)methyl)pyridin-2-yl)-[1,1′-biphenyl]-3-yl)-2-((3-hydroxy-3-methylazetidin-1-yl)methyl)-3-methylpyrrolo[2,1-f][1,2,4]triazin-4(3H)-one;
(S)-6-(2,2′-dichloro-3′-(4-fluoro-6-methoxy-5-((((5-oxopyrrolidin-2-yl)methyl)amino)methyl)pyridin-2-yl)-[1,1′-biphenyl]-3-yl)-2-(((2-hydroxyethyl)amino)methyl)-3-methylpyrrolo[2,1-f][1,2,4]triazin-4(3H)-one;
(S)-1-((6-(2,2′-dichloro-3′-(6-methoxy-5-(((((S)-5-oxopyrrolidin-2-yl)methyl)amino)methyl)pyridin-2-yl)-[1,1′-biphenyl]-3-yl)-3-methyl-4-oxo-3,4-dihydropyrrolo[2,1-f][1,2,4]triazin-2-yl)methyl)pyrrolidine-3-carboxylic acid;
(S)-1-(((6-(2,2′-dichloro-3′-(6-methoxy-5-((((5-oxopyrrolidin-2-yl)methyl)amino)methyl)pyridin-2-yl)-[1,1′-biphenyl]-3-yl)-3-methyl-4-oxo-3,4-dihydropyrrolo[2,1-f][1,2,4]triazin-2-yl)methyl)amino)cyclopropane-1-carboxylic acid;
(S)-3-(((6-(2,2′-dichloro-3′-(6-methoxy-5-((((5-oxopyrrolidin-2-yl)methyl)amino)methyl)pyridin-2-yl)-[1,1′-biphenyl]-3-yl)-3-methyl-4-oxo-3,4-dihydropyrrolo[2,1-f][1,2,4]triazin-2-yl)methyl)amino)-2,2-dimethylpropanoic acid;
methyl ((6-(2,2′-dichloro-3′-(6-methoxy-5-(((((S)-5-oxopyrrolidin-2-yl)methyl)amino)methyl)pyridin-2-yl)-[1,1′-biphenyl]-3-yl)-3-methyl-4-oxo-3,4-dihydropyrrolo[2,1-f][1,2,4]triazin-2-yl)methyl)-D-alaninate;
(S)-1-((6-(2,2′-dichloro-3′-(6-methoxy-5-((((5-oxopyrrolidin-2-yl)methyl)amino)methyl)pyridin-2-yl)-[1,1′-biphenyl]-3-yl)-3-methyl-4-oxo-3,4-dihydropyrrolo[2,1-f][1,2,4]triazin-2-yl)methyl)azetidine-3-carboxylic acid;
(5S)-5-[[[6-[2-chloro-3-[2-chloro-3-[2-[[[(2S)-5-oxopyrrolidin-2-yl]methylamino]methyl]-[1,2,4]triazolo[1,5-a]pyrimidin-6-yl]phenyl]phenyl]-2-methoxy-3-pyridyl]methylamino] methyl]pyrrolidin-2-one;
(2R)-2-[[6-[2-chloro-3-[2-chloro-3-[6-methoxy-5-[[[(2S)-5-oxopyrrolidin-2-yl]methylamino] methyl]-2-pyridyl]phenyl]phenyl]-3-methyl-4-oxo-pyrrolo[2,1-f][1,2,4]triazin-2-yl]methylamino]propanoic acid;
(S)-6-(2,2′-Dichloro-3′-(4-fluoro-6-methoxy-5-((((5-oxopyrrolidin-2-yl)methyl)amino)methyl)pyridin-2-yl)-[1,1′-biphenyl]-3-yl)-2-(((3-fluoropropyl)amino)methyl)-3-methylpyrrolo[2,1-f][1,2,4]triazin-4(3H)-one;
(S)-5-((6-(2,2′-Dichloro-3′-(3-fluoro-6-methoxy-5-(((((S)-5-oxopyrrolidin-2-yl)methyl)amino)methyl)pyridin-2-yl)-[1,1′-biphenyl]-3-yl)-8-methoxy-3,4-dihydroisoquinolin-2(1H)-yl)methyl)pyrrolidin-2-one;
2-(2,2′-Dichloro-3′-(6-methoxy-5-(((((S)-5-oxopyrrolidin-2-yl)methyl)amino)methyl)pyridin-2-yl)-[1,1′-biphenyl]-3-yl)-1-methyl-5-(((((S)-5-oxopyrrolidin-2-yl)methyl)amino)methyl)pyrazolo[1,5-a]pyrimidin-7(1H)-one;
6-(2,2′-Dichloro-3′-(3-fluoro-6-methoxy-5-(((((S)-5-oxopyrrolidin-2-yl)methyl)amino)methyl)pyridin-2-yl)-[1,1′-biphenyl]-3-yl)-2-((((1r,3r)-3-hydroxy-3-methylcyclobutyl)amino)methyl)-3-methylpyrrolo[2,1-f][1,2,4]triazin-4(3H)-one;
(S)-Isopropyl 2-(((6-(2,2′-dichloro-3′-(4-chloro-6-methoxy-5-((((5-oxopyrrolidin-2-yl)methyl)amino)methyl)pyridin-2-yl)-[1,1′-biphenyl]-3-yl)-3-methyl-4-oxo-3,4-dihydropyrrolo[2,1-f][1,2,4]triazin-2-yl)methyl)amino)-2-methylpropanoate;
(3S)-1-[[6-[2-Chloro-3-[2-chloro-3-[6-methoxy-5-[[[(2S)-5-oxopyrrolidin-2-yl]methylamino]methyl]-2-pyridyl]phenyl]phenyl]-3-methyl-4-oxo-pyrrolo[2,1-f][1,2,4]triazin-2-yl]methyl]piperidine-3-carboxylic acid;
(S)-2-(2,2′-Dichloro-3′-(6-methoxy-5-((((5-oxopyrrolidin-2-yl)methyl)amino)methyl)pyridin-2-yl)-[1,1′-biphenyl]-3-yl)-5-(((2-hydroxyethyl)amino)methyl)-1-methylpyrazolo[1,5-a]pyrimidin-7(1H)-one;
(5S)-5-[[[6-[3-[3-[2-[[(1-Acetyl-4-piperidyl)amino]methyl]-[1,2,4]triazolo[1,5-a]pyrimidin-6-yl]-2-chloro-phenyl]-2-chloro-phenyl]-2-methoxy-3-pyridyl]methylamino]methyl] pyrrolidin-2-one;
3-[[6-[2-Chloro-3-[2-chloro-3-[6-methoxy-5-[[[(2S)-5-oxopyrrolidin-2-yl]methylamino]methyl]-2-pyridyl]phenyl]phenyl]-3-methyl-4-oxo-pyrrolo[2,1-f][1,2,4]triazin-2-yl]methylamino]propanoic acid;
(S)-2-(2,2′-Dichloro-3′-(6-methoxy-5-((((5-oxopyrrolidin-2-yl)methyl)amino)methyl)pyridin-2-yl)-[1,1′-biphenyl]-3-yl)-5-((6-hydroxy-2-azaspiro[3.3]heptan-2-yl)methyl)-1-methylpyrazolo[1,5-a]pyrimidin-7(1H)-one.
In certain embodiments, the compound of formula (Ia) or (Ib) is selected from the group consisting of:
5-((4-chloro-2-(((2-hydroxyethyl)amino)methyl)-5-((3′-(3-(((2-hydroxyethyl)amino)methyl)-4-oxo-4H-pyrido[1,2-a]pyrimidin-8-yl)-2-methyl-[1,1′-biphenyl]-3-yl)methoxy)phenoxy)methyl)nicotinamide;
5-(((2-hydroxyethyl)amino)methyl)-N-(3′-(3-(((2-hydroxyethyl)amino)methyl)-4-oxo-4H-pyrido[1,2-a]pyrimidin-8-yl)-2-methyl-[1,1′-biphenyl]-3-yl)picolinamide;
5-((4-chloro-2-(((2-hydroxyethyl)amino)methyl)-5-((3′-(3-(((2-hydroxyethyl)amino)methyl)-4-oxo-4H-pyrido[1,2-a]pyrimidin-8-yl)-2,2′-dimethyl-[1,1′-biphenyl]-3-yl)methoxy)phenoxy)methyl)nicotinonitrile;
5-((4-chloro-2-(((2-hydroxyethyl)amino)methyl)-5-((3′-(3-(((2-hydroxyethyl)amino)methyl)-4-oxo-4H-pyrido[1,2-a]pyrimidin-8-yl)-2-methyl-[1,1′-biphenyl]-3-yl)methoxy)phenoxy)methyl)nicotinonitrile;
5-((4-chloro-2-(((2-hydroxyethyl)amino)methyl)-5-((3′-(7-(((2-hydroxyethyl)amino)methyl)-4-oxoquinazolin-3(4H)-yl)-2-methyl-[1,1′-biphenyl]-3-yl)methoxy)phenoxy)methyl)nicotinonitrile;
5-((4-chloro-2-(((2-hydroxyethyl)amino)methyl)-5-((3′-(7-(((2-hydroxyethyl)amino)methyl)-4-oxoquinazolin-3(4H)-yl)-2,2′-dimethyl-[1,1′-biphenyl]-3-yl)methoxy)phenoxy)methyl)nicotinonitrile;
(S)-2-(((8-(3′-((4-((((S)-2-carboxy-1-hydroxypropan-2-yl)amino)methyl)-2-chloro-5-((5-cyanopyridin-3-yl)methoxy)phenoxy)methyl)-2,2′-dimethyl-[1,1′-biphenyl]-3-yl)-4-oxo-4H-pyrido[1,2-a]pyrimidin-3-yl)methyl)amino)-3-hydroxy-2-methylpropanoic acid;
(S)-2-(((8-(3′-((4-((((R)-2-carboxy-1-hydroxypropan-2-yl)amino)methyl)-2-chloro-5-((5-cyanopyridin-3-yl)methoxy)phenoxy)methyl)-2,2′-dimethyl-[1,1′-biphenyl]-3-yl)-4-oxo-4H-pyrido[1,2-a]pyrimidin-3-yl)methyl)amino)-3-hydroxy-2-methylpropanoic acid;
(R)-2-(((8-(3′-((4-((((R)-2-carboxy-1-hydroxypropan-2-yl)amino)methyl)-2-chloro-5-((5-cyanopyridin-3-yl)methoxy)phenoxy)methyl)-2,2′-dimethyl-[1,1′-biphenyl]-3-yl)-4-oxo-4H-pyrido[1,2-a]pyrimidin-3-yl)methyl)amino)-3-hydroxy-2-methylpropanoic acid;
(R)-2-(((8-(3′-((4-((((R)-2-carboxy-1-hydroxypropan-2-yl)amino)methyl)-2-chloro-5-((5-cyanopyridin-3-yl)methoxy)phenoxy)methyl)-2,2′-dimethyl-[1,1′-biphenyl]-3-yl)-4-oxo-4H-pyrido[1,2-a]pyrimidin-3-yl)methyl)amino)-3-hydroxy-2-methylpropanoic acid;
5-((4-chloro-5-((3′-(1-(2-((2-hydroxyethyl)amino)ethyl)-4-methyl-2,5-dioxo-2,3,4,5-tetrahydro-1H-benzo[e][1,4]diazepin-7-yl)-2,2′-dimethyl-[1,1′-biphenyl]-3-yl)methoxy)-2-(((2-hydroxyethyl)amino)methyl)phenoxy)methyl)nicotinonitrile;
5-((4-chloro-2-(((2-hydroxyethyl)amino)methyl)-5-((3′-(3-(((2-hydroxyethyl)amino)methyl)-4-oxo-4H-quinolizin-8-yl)-2,2′-dimethyl-[1,1′-biphenyl]-3-yl)methoxy)phenoxy)methyl)nicotinonitrile;
(S)-2-(((8-(3′-((4-((((S)-2-carboxy-1-hydroxypropan-2-yl)amino)methyl)-2-chloro-5-((5-cyanopyridin-3-yl)methoxy)phenoxy)methyl)-2,2′-dimethyl-[1,1′-biphenyl]-3-yl)-4-oxo-4H-pyrido[1,2-a]pyrimidin-3-yl)methyl)amino)-3-hydroxy-2-methylpropanoic acid;
(S)-2-(((8-(3′-((4-((((R)-2-carboxy-1-hydroxypropan-2-yl)amino)methyl)-2-chloro-5-((5-cyanopyridin-3-yl)methoxy)phenoxy)methyl)-2,2′-dimethyl-[1,1′-biphenyl]-3-yl)-4-oxo-4H-pyrido[1,2-a]pyrimidin-3-yl)methyl)amino)-3-hydroxy-2-methylpropanoic acid;
(R)-2-(((8-(3′-((4-((((S)-2-carboxy-1-hydroxypropan-2-yl)amino)methyl)-2-chloro-5-((5-cyanopyridin-3-yl)methoxy)phenoxy)methyl)-2,2′-dimethyl-[1,1′-biphenyl]-3-yl)-4-oxo-4H-pyrido[1,2-a]pyrimidin-3-yl)methyl)amino)-3-hydroxy-2-methylpropanoic acid;
(R)-2-(((8-(3′-((4-((((R)-2-carboxy-1-hydroxypropan-2-yl)amino)methyl)-2-chloro-5-((5-cyanopyridin-3-yl)methoxy)phenoxy)methyl)-2,2′-dimethyl-[1,1′-biphenyl]-3-yl)-4-oxo-4H-pyrido[1,2-a]pyrimidin-3-yl)methyl)amino)-3-hydroxy-2-methylpropanoic acid;
(S)-1-((8-(3′-((4-(((S)-2-carboxypiperidin-1-yl)methyl)-2-chloro-5-((5-cyanopyridin-3-yl)methoxy)phenoxy)methyl)-2,2′-dimethyl-[1,1′-biphenyl]-3-yl)-4-oxo-4H-pyrido[1,2-a]pyrimidin-3-yl)methyl)piperidine-2-carboxylic acid;
(S)-1-((8-(3′-((4-(((R)-2-carboxypiperidin-1-yl)methyl)-2-chloro-5-((5-cyanopyridin-3-yl)methoxy)phenoxy)methyl)-2,2′-dimethyl-[1,1′-biphenyl]-3-yl)-4-oxo-4H-pyrido[1,2-a]pyrimidin-3-yl)methyl)piperidine-2-carboxylic acid;
(R)-1-((8-(3′-((4-(((R)-2-carboxypiperidin-1-yl)methyl)-2-chloro-5-((5-cyanopyridin-3-yl)methoxy)phenoxy)methyl)-2,2′-dimethyl-[1,1′-biphenyl]-3-yl)-4-oxo-4H-pyrido[1,2-a]pyrimidin-3-yl)methyl)piperidine-2-carboxylic acid;
(R)-1-((8-(3′-((4-(((S)-2-carboxypiperidin-1-yl)methyl)-2-chloro-5-((5-cyanopyridin-3-yl)methoxy)phenoxy)methyl)-2,2′-dimethyl-[1,1′-biphenyl]-3-yl)-4-oxo-4H-pyrido[1,2-a]pyrimidin-3-yl)methyl)piperidine-2-carboxylic acid;
5-((4-chloro-5-((2,2′-dimethyl-3′-(4-oxo-3-(((((S)-5-oxopyrrolidin-2-yl)methyl)amino)methyl)-4H-pyrido[1,2-a]pyrimidin-8-yl)-[1,1′-biphenyl]-3-yl)methoxy)-2-(((((S)-5-oxopyrrolidin-2-yl)methyl)amino)methyl)phenoxy)methyl)nicotinonitrile;
5-((4-chloro-5-((2,2′-dimethyl-3′-(4-oxo-3-(((((S)-5-oxopyrrolidin-2-yl)methyl)amino)methyl)-4H-pyrido[1,2-a]pyrimidin-8-yl)-[1,1′-biphenyl]-3-yl)methoxy)-2-(((((R)-5-oxopyrrolidin-2-yl)methyl)amino)methyl)phenoxy)methyl)nicotinonitrile;
5-((4-chloro-5-((2,2′-dimethyl-3′-(4-oxo-3-(((((R)-5-oxopyrrolidin-2-yl)methyl)amino)methyl)-4H-pyrido[1,2-a]pyrimidin-8-yl)-[1,1′-biphenyl]-3-yl)methoxy)-2-(((((R)-5-oxopyrrolidin-2-yl)methyl)amino)methyl)phenoxy)methyl)nicotinonitrile;
5-((4-chloro-5-((2,2′-dimethyl-3′-(4-oxo-3-(((((R)-5-oxopyrrolidin-2-yl)methyl)amino)methyl)-4H-pyrido[1,2-a]pyrimidin-8-yl)-[1,1′-biphenyl]-3-yl)methoxy)-2-(((((S)-5-oxopyrrolidin-2-yl)methyl)amino)methyl)phenoxy)methyl)nicotinonitrile;
5-((4-chloro-5-((2-chloro-3′-(3-(((2-hydroxyethyl)amino)methyl)-4-oxo-4H-pyrido[1,2-a]pyrimidin-8-yl)-2′-methyl-[1,1′-biphenyl]-3-yl)methoxy)-2-(((2-hydroxyethyl)amino)methyl)phenoxy)methyl)nicotinonitrile;
8,8′-(2,2′-dimethyl-[1,1′-biphenyl]-3,3′-diyl)bis(3-(((2-hydroxyethyl)amino)methyl)-4H-pyrido[1,2-a]pyrimidin-4-one);
5-((4-chloro-5-((2-cyano-3′-(3-(((2-hydroxyethyl)amino)methyl)-4-oxo-4H-pyrido[1,2-a]pyrimidin-8-yl)-2′-methyl-[1,1′-biphenyl]-3-yl)methoxy)-2-(((2-hydroxyethyl)amino)methyl)phenoxy)methyl)nicotinonitrile;
5-((4-chloro-2-(((2-hydroxyethyl)amino)methyl)-5-((3′-(8-(((2-hydroxyethyl)amino)methyl)-4-oxo-4H-pyrido[1,2-a]pyrimidin-3-yl)-2,2′-dimethyl-[1,1′-biphenyl]-3-yl)methoxy)phenoxy)methyl)nicotinonitrile;
5-((4-chloro-2-(((2-hydroxyethyl)amino)methyl)-5-(((3′-(3-(((2-hydroxyethyl)amino)methyl)-4-oxo-4H-pyrido[1,2-a]pyrimidin-8-yl)-2,2′-dimethyl-[1,1′-biphenyl]-3-yl)oxy)methyl)phenoxy)methyl)nicotinonitrile;
5-((4-chloro-5-(((2,2′-dimethyl-3′-(4-oxo-3-(((((S)-5-oxopyrrolidin-2-yl)methyl)amino)methyl)-4H-pyrido[1,2-a]pyrimidin-8-yl)-[1,1′-biphenyl]-3-yl)oxy)methyl)-2-(((((S)-5-oxopyrrolidin-2-yl)methyl)amino)methyl)phenoxy)methyl)nicotinonitrile;
5-((4-chloro-5-(((2,2′-dimethyl-3′-(4-oxo-3-(((((R)-5-oxopyrrolidin-2-yl)methyl)amino)methyl)-4H-pyrido[1,2-a]pyrimidin-8-yl)-[1,1′-biphenyl]-3-yl)oxy)methyl)-2-(((((S)-5-oxopyrrolidin-2-yl)methyl)amino)methyl)phenoxy)methyl)nicotinonitrile;
5-((4-chloro-5-(((2,2′-dimethyl-3′-(4-oxo-3-(((((S)-5-oxopyrrolidin-2-yl)methyl)amino)methyl)-4H-pyrido[1,2-a]pyrimidin-8-yl)-[1,1′-biphenyl]-3-yl)oxy)methyl)-2-(((((R)-5-oxopyrrolidin-2-yl)methyl)amino)methyl)phenoxy)methyl)nicotinonitrile;
5-((4-chloro-5-(((2,2′-dimethyl-3′-(4-oxo-3-(((((R)-5-oxopyrrolidin-2-yl)methyl)amino)methyl)-4H-pyrido[1,2-a]pyrimidin-8-yl)-[1,1′-biphenyl]-3-yl)oxy)methyl)-2-(((((R)-5-oxopyrrolidin-2-yl)methyl)amino)methyl)phenoxy)methyl)nicotinonitrile;
5-((4-chloro-5-((2,2′-dimethyl-3′-(4-methyl-2,5-dioxo-1-(2-((((S)-5-oxopyrrolidin-2-yl)methyl)amino)ethyl)-2,3,4,5-tetrahydro-1H-benzo[e][1,4]diazepin-7-yl)-[1,1′-biphenyl]-3-yl)methoxy)-2-(((((S)-5-oxopyrrolidin-2-yl)methyl)amino)methyl)phenoxy)methyl)nicotinonitrile;
5-((4-chloro-5-((2,2′-dimethyl-3′-(4-methyl-2,5-dioxo-1-(2-((((R)-5-oxopyrrolidin-2-yl)methyl)amino)ethyl)-2,3,4,5-tetrahydro-1H-benzo[e][1,4]diazepin-7-yl)-[1,1′-biphenyl]-3-yl)methoxy)-2-(((((S)-5-oxopyrrolidin-2-yl)methyl)amino)methyl)phenoxy)methyl)nicotinonitrile;
5-((4-chloro-5-((2,2′-dimethyl-3′-(4-methyl-2,5-dioxo-1-(2-((((S)-5-oxopyrrolidin-2-yl)methyl)amino)ethyl)-2,3,4,5-tetrahydro-1H-benzo[e][1,4]diazepin-7-yl)-[1,1′-biphenyl]-3-yl)methoxy)-2-(((((R)-5-oxopyrrolidin-2-yl)methyl)amino)methyl)phenoxy)methyl)nicotinonitrile;
5-((4-chloro-5-((2,2′-dimethyl-3′-(4-methyl-2,5-dioxo-1-(2-((((R)-5-oxopyrrolidin-2-yl)methyl)amino)ethyl)-2,3,4,5-tetrahydro-1H-benzo[e][1,4]diazepin-7-yl)-[1,1′-biphenyl]-3-yl)methoxy)-2-(((((R)-5-oxopyrrolidin-2-yl)methyl)amino)methyl)phenoxy)methyl)nicotinonitrile;
(Z)-8-(3′-(2-fluoro-2-(5-(((2-hydroxyethyl)amino)methyl)pyridin-2-yl)vinyl)-2,2′-dimethyl-[1,1′-biphenyl]-3-yl)-3-(((2-hydroxyethyl)amino)methyl)-4H-pyrido[1,2-a]pyrimidin-4-one;
(R)-5-((4-chloro-5-((3′-(5-((2-hydroxyethyl)amino)-1-methyl-2-oxo-2,3,4,5-tetrahydro-1H-benzo[b]azepin-8-yl)-2,2′-dimethyl-[1,1′-biphenyl]-3-yl)methoxy)-2-((((2-hydroxyethyl)amino)methyl)phenoxy)methyl)nicotinonitrile;
(S)-5-((4-chloro-5-((3′-(5-((2-hydroxyethyl)amino)-1-methyl-2-oxo-2,3,4,5-tetrahydro-1H-benzo[b]azepin-8-yl)-2,2′-dimethyl-[1,1′-biphenyl]-3-yl)methoxy)-2-((((2-hydroxyethyl)amino)methyl)phenoxy)methyl)nicotinonitrile;
5-((4-chloro-2-(((2-hydroxyethyl)amino)methyl)-5-((3′-(4-(((2-hydroxyethyl)amino)methyl)-2-oxopyridin-1(2H)-yl)-2,2′-dimethyl-[1,1′-biphenyl]-3-yl)methoxy)phenoxy)methyl)nicotinonitrile;
5-((4-chloro-5-((2,2′-dimethyl-3′-(4-oxo-3-(((((S)-5-oxopyrrolidin-2-yl)methyl)amino)methyl)-4H-quinolizin-8-yl)-[1,1′-biphenyl]-3-yl)methoxy)-2-(((((S)-5-oxopyrrolidin-2-yl)methyl)amino)methyl)phenoxy)methyl)nicotinonitrile;
5-((4-chloro-5-((2,2′-dimethyl-3′-(4-oxo-3-(((((R)-5-oxopyrrolidin-2-yl)methyl)amino)methyl)-4H-quinolizin-8-yl)-[1,1′-biphenyl]-3-yl)methoxy)-2-(((((S)-5-oxopyrrolidin-2-yl)methyl)amino)methyl)phenoxy)methyl)nicotinonitrile;
5-((4-chloro-5-((2,2′-dimethyl-3′-(4-oxo-3-(((((S)-5-oxopyrrolidin-2-yl)methyl)amino)methyl)-4H-quinolizin-8-yl)-[1,1′-biphenyl]-3-yl)methoxy)-2-(((((R)-5-oxopyrrolidin-2-yl)methyl)amino)methyl)phenoxy)methyl)nicotinonitrile;
5-((4-chloro-5-((2,2′-dimethyl-3′-(4-oxo-3-(((((R)-5-oxopyrrolidin-2-yl)methyl)amino)methyl)-4H-quinolizin-8-yl)-[1,1′-biphenyl]-3-yl)methoxy)-2-(((((R)-5-oxopyrrolidin-2-yl)methyl)amino)methyl)phenoxy)methyl)nicotinonitrile;
5-((4-chloro-5-((2′-cyano-3′-(3-(((2-hydroxyethyl)amino)methyl)-4-oxo-4H-pyrido[1,2-a]pyrimidin-8-yl)-2-methyl-[1,1′-biphenyl]-3-yl)methoxy)-2-(((2-hydroxyethyl)amino)methyl)phenoxy)methyl)nicotinonitrile;
5-((4-chloro-5-((3′-(1-(2-(3-(hydroxymethyl)azetidin-1-yl)ethyl)-4-methyl-2,5-dioxo-2,3,4,5-tetrahydro-1H-benzo[e][1,4]diazepin-7-yl)-2,2′-dimethyl-[1,1′-biphenyl]-3-yl)methoxy)-2-((3-(hydroxymethyl)azetidin-1-yl)methyl)phenoxy)methyl)nicotinonitrile;
5-((4-chloro-5-(((2,2′-dimethyl-3′-(4-methyl-2,5-dioxo-1-(2-((((S)-5-oxopyrrolidin-2-yl)methyl)amino)ethyl)-2,3,4,5-tetrahydro-1H-benzo[e][1,4]diazepin-7-yl)-[1,1′-biphenyl]-3-yl)oxy)methyl)-2-(((((S)-5-oxopyrrolidin-2-yl)methyl)amino)methyl)phenoxy)methyl)nicotinonitrile;
5-((4-chloro-5-(((2,2′-dimethyl-3′-(4-methyl-2,5-dioxo-1-(2-((((R)-5-oxopyrrolidin-2-yl)methyl)amino)ethyl)-2,3,4,5-tetrahydro-1H-benzo[e][1,4]diazepin-7-yl)-[1,1′-biphenyl]-3-yl)oxy)methyl)-2-(((((S)-5-oxopyrrolidin-2-yl)methyl)amino)methyl)phenoxy)methyl)nicotinonitrile;
5-((4-chloro-5-(((2,2′-dimethyl-3′-(4-methyl-2,5-dioxo-1-(2-((((S)-5-oxopyrrolidin-2-yl)methyl)amino)ethyl)-2,3,4,5-tetrahydro-1H-benzo[e][1,4]diazepin-7-yl)-[1,1′-biphenyl]-3-yl)oxy)methyl)-2-(((((R)-5-oxopyrrolidin-2-yl)methyl)amino)methyl)phenoxy)methyl)nicotinonitrile;
5-((4-chloro-5-(((2,2′-dimethyl-3′-(4-methyl-2,5-dioxo-1-(2-((((R)-5-oxopyrrolidin-2-yl)methyl)amino)ethyl)-2,3,4,5-tetrahydro-1H-benzo[e][1,4]diazepin-7-yl)-[1,1′-biphenyl]-3-yl)oxy)methyl)-2-(((((R)-5-oxopyrrolidin-2-yl)methyl)amino)methyl)phenoxy)methyl)nicotinonitrile;
5-((4-chloro-5-((2,2′-dimethyl-3′-(4-oxo-3-(((((R)-5-oxopyrrolidin-2-yl)methyl)amino)methyl)-4H-quinolizin-8-yl)-[1,1′-biphenyl]-3-yl)methoxy)-2-(((((R)-5-oxopyrrolidin-2-yl)methyl)amino)methyl)phenoxy)methyl)nicotinonitrile;
5-((4-chloro-5-((2,2′-dimethyl-3′-(4-oxo-3-(((((R)-5-oxopyrrolidin-2-yl)methyl)amino)methyl)-4H-quinolizin-8-yl)-[1,1′-biphenyl]-3-yl)methoxy)-2-(((((S)-5-oxopyrrolidin-2-yl)methyl)amino)methyl)phenoxy)methyl)nicotinonitrile;
5-((4-chloro-5-((2,2′-dimethyl-3′-(4-oxo-3-(((((S)-5-oxopyrrolidin-2-yl)methyl)amino)methyl)-4H-quinolizin-8-yl)-[1,1′-biphenyl]-3-yl)methoxy)-2-(((((R)-5-oxopyrrolidin-2-yl)methyl)amino)methyl)phenoxy)methyl)nicotinonitrile;
5-((4-chloro-5-((2,2′-dimethyl-3′-(4-oxo-3-(((((S)-5-oxopyrrolidin-2-yl)methyl)amino)methyl)-4H-quinolizin-8-yl)-[1,1′-biphenyl]-3-yl)methoxy)-2-(((((S)-5-oxopyrrolidin-2-yl)methyl)amino)methyl)phenoxy)methyl)nicotinonitrile;
1-((8-(3′-((4-((3-carboxyazetidin-1-yl)methyl)-2-chloro-5-((5-cyanopyridin-3-20 yl)methoxy)phenoxy)methyl)-2,2′-dimethyl-[1,1′-biphenyl]-3-yl)-4-oxo-4H-quinolizin-3-yl)methyl)azetidine-3-carboxylic acid;
(S)-1-(4-((3′-(1-(2-((S)-3-carboxypyrrolidin-1-yl)ethyl)-4-methyl-2,5-dioxo-2,3,4,5-tetrahydro-1H-benzo[e][1,4]diazepin-7-yl)-2,2′-dimethyl-[1,1′-biphenyl]-3-yl)methoxy)-5-chloro-2-((5-cyanopyridin-3-yl)methoxy)benzyl)pyrrolidine-3-carboxylic acid;
(R)-1-(4-((3′-(1-(2-((S)-3-carboxypyrrolidin-1-yl)ethyl)-4-methyl-2,5-dioxo-2,3,4,5-tetrahydro-1H-benzo[e][1,4]diazepin-7-yl)-2,2′-dimethyl-[1,1′-biphenyl]-3-yl)methoxy)-5-chloro-2-((5-cyanopyridin-3-yl)methoxy)benzyl)pyrrolidine-3-carboxylic acid;
(S)-1-(4-((3′-(1-(2-((R)-3-carboxypyrrolidin-1-yl)ethyl)-4-methyl-2,5-dioxo-2,3,4,5-tetrahydro-1H-benzo[e][1,4]diazepin-7-yl)-2,2′-dimethyl-[1,1′-biphenyl]-3-yl)methoxy)-5-chloro-2-((5-cyanopyridin-3-yl)methoxy)benzyl)pyrrolidine-3-carboxylic acid;
(R)-1-(4-((3′-(1-(2-((R)-3-carboxypyrrolidin-1-yl)ethyl)-4-methyl-2,5-dioxo-2,3,4,5-tetrahydro-1H-benzo[e][1,4]diazepin-7-yl)-2,2′-dimethyl-[1,1′-biphenyl]-3-yl)methoxy)-5-chloro-2-((5-cyanopyridin-3-yl)methoxy)benzyl)pyrrolidine-3-carboxylic acid;
8-(3′-(6-methoxy-5-(((((S)-5-oxopyrrolidin-2-yl)methyl)amino)methyl)pyridin-2-yl)-2,2′-dimethyl-[1,1′-biphenyl]-3-yl)-3-(((((S)-5-oxopyrrolidin-2-yl)methyl)amino)methyl)-4H-pyrido[1,2-a]pyrimidin-4-one;
8-(3′-(6-methoxy-5-(((((R)-5-oxopyrrolidin-2-yl)methyl)amino)methyl)pyridin-2-yl)-2,2′-dimethyl-[1,1′-biphenyl]-3-yl)-3-(((((S)-5-oxopyrrolidin-2-yl)methyl)amino)methyl)-4H-pyrido[1,2-a]pyrimidin-4-one;
8-(3′-(6-methoxy-5-(((((S)-5-oxopyrrolidin-2-yl)methyl)amino)methyl)pyridin-2-yl)-2,2′-dimethyl-[1,1′-biphenyl]-3-yl)-3-(((((R)-5-oxopyrrolidin-2-yl)methyl)amino)methyl)-4H-pyrido[1,2-a]pyrimidin-4-one;
8-(3′-(6-methoxy-5-(((((R)-5-oxopyrrolidin-2-yl)methyl)amino)methyl)pyridin-2-yl)-2,2′-dimethyl-[1,1′-biphenyl]-3-yl)-3-(((((R)-5-oxopyrrolidin-2-yl)methyl)amino)methyl)-4H-pyrido[1,2-a]pyrimidin-4-one;
8-(2′-chloro-3′-(6-methoxy-5-(((((S)-5-oxopyrrolidin-2-yl)methyl)amino)methyl)pyridin-2-yl)-2-methyl-[1,1′-biphenyl]-3-yl)-3-(((((S)-5-oxopyrrolidin-2-yl)methyl)amino)methyl)-4H-pyrido[1,2-a]pyrimidin-4-one;
8-(2′-chloro-3′-(6-methoxy-5-(((((R)-5-oxopyrrolidin-2-yl)methyl)amino)methyl)pyridin-2-yl)-2-methyl-[1,1′-biphenyl]-3-yl)-3-(((((S)-5-oxopyrrolidin-2-yl)methyl)amino)methyl)-4H-pyrido[1,2-a]pyrimidin-4-one;
8-(2′-chloro-3′-(6-methoxy-5-(((((S)-5-oxopyrrolidin-2-yl)methyl)amino)methyl)pyridin-2-yl)-2-methyl-[1,1′-biphenyl]-3-yl)-3-(((((R)-5-oxopyrrolidin-2-yl)methyl)amino)methyl)-4H-pyrido[1,2-a]pyrimidin-4-one;
8-(2′-chloro-3′-(6-methoxy-5-(((((R)-5-oxopyrrolidin-2-yl)methyl)amino)methyl)pyridin-2-yl)-2-methyl-[1,1′-biphenyl]-3-yl)-3-(((((R)-5-oxopyrrolidin-2-yl)methyl)amino)methyl)-4H-pyrido[1,2-a]pyrimidin-4-one;
8-(2,2′-dichloro-3′-(6-methoxy-5-(((((S)-5-oxopyrrolidin-2-yl)methyl)amino)methyl)pyridin-2-yl)-[1,1′-biphenyl]-3-yl)-3-(((((S)-5-oxopyrrolidin-2-yl)methyl)amino)methyl)-4H-pyrido[1,2-a]pyrimidin-4-one;
8-(2,2′-dichloro-3′-(6-methoxy-5-(((((R)-5-oxopyrrolidin-2-yl)methyl)amino)methyl)pyridin-2-yl)-[1,1′-biphenyl]-3-yl)-3-(((((S)-5-oxopyrrolidin-2-yl)methyl)amino)methyl)-4H-pyrido[1,2-a]pyrimidin-4-one;
8-(2,2′-dichloro-3′-(6-methoxy-5-(((((S)-5-oxopyrrolidin-2-yl)methyl)amino)methyl)pyridin-2-yl)-[1,1′-biphenyl]-3-yl)-3-(((((R)-5-oxopyrrolidin-2-yl)methyl)amino)methyl)-4H-pyrido[1,2-a]pyrimidin-4-one;
8-(2,2′-dichloro-3′-(6-methoxy-5-(((((R)-5-oxopyrrolidin-2-yl)methyl)amino)methyl)pyridin-2-yl)-[1,1′-biphenyl]-3-yl)-3-(((((R)-5-oxopyrrolidin-2-yl)methyl)amino)methyl)-4H-pyrido[1,2-a]pyrimidin-4-one;
2-(((S)-7-(2,2′-dichloro-3′-((R)-5-((2-hydroxyethyl)amino)-8-methyl-5,6,7,8-tetrahydro-1,8-naphthyridin-2-yl)-[1,1′-biphenyl]-3-yl)-1-methyl-1,2,3,4-tetrahydro-1,8-naphthyridin-4-yl)amino)ethan-1-ol;
2-(((S)-7-(2,2′-dichloro-3′-((S)-5-((2-hydroxyethyl)amino)-8-methyl-5,6,7,8-tetrahydro-1,8-naphthyridin-2-yl)-[1,1′-biphenyl]-3-yl)-1-methyl-1,2,3,4-tetrahydro-1,8-naphthyridin-4-yl)amino)ethan-1-ol;
2-(((R)-7-(2,2′-dichloro-3′-((R)-5-((2-hydroxyethyl)amino)-8-methyl-5,6,7,8-tetrahydro-1,8-naphthyridin-2-yl)-[1,1′-biphenyl]-3-yl)-1-methyl-1,2,3,4-tetrahydro-1,8-naphthyridin-4-yl)amino)ethan-1-ol;
2-(((R)-7-(2,2′-dichloro-3′-((S)-5-((2-hydroxyethyl)amino)-8-methyl-5,6,7,8-tetrahydro-1,8-naphthyridin-2-yl)-[1,1′-biphenyl]-3-yl)-1-methyl-1,2,3,4-tetrahydro-1,8-naphthyridin-4-yl)amino)ethan-1-ol;
2,2′-(2,2′-dichloro-[1,1′-biphenyl]-3,3′-diyl)bis(9-methyl-7-(3-(methylamino)propoxy)-4H-pyrido[1,2-a]pyrimidin-4-one);
8-(2′-chloro-3′-(6-methoxy-5-(((((S)-5-oxopyrrolidin-2-yl)methyl)amino)methyl)pyrazin-2-yl)-2-methyl-[1,1′-biphenyl]-3-yl)-3-(((((S)-5-oxopyrrolidin-2-yl)methyl)amino)methyl)-4H-pyrido[1,2-a]pyrimidin-4-one;
8-(2′-chloro-3′-(6-methoxy-5-(((((R)-5-oxopyrrolidin-2-yl)methyl)amino)methyl)pyrazin-2-yl)-2-methyl-[1,1′-biphenyl]-3-yl)-3-(((((S)-5-oxopyrrolidin-2-yl)methyl)amino)methyl)-4H-pyrido[1,2-a]pyrimidin-4-one;
8-(2′-chloro-3′-(6-methoxy-5-(((((S)-5-oxopyrrolidin-2-yl)methyl)amino)methyl)pyrazin-2-yl)-2-methyl-[1,1′-biphenyl]-3-yl)-3-(((((R)-5-oxopyrrolidin-2-yl)methyl)amino)methyl)-4H-pyrido[1,2-a]pyrimidin-4-one;
8-(2′-chloro-3′-(6-methoxy-5-(((((R)-5-oxopyrrolidin-2-yl)methyl)amino)methyl)pyrazin-2-yl)-2-methyl-[1,1′-biphenyl]-3-yl)-3-(((((R)-5-oxopyrrolidin-2-yl)methyl)amino)methyl)-4H-pyrido[1,2-a]pyrimidin-4-one;
8-(2,2′-dimethyl-3′-(4-oxo-3-(((((R)-5-oxopyrrolidin-2-yl)methyl)amino)methyl)-4H-pyrido[1,2-a]pyrimidin-8-yl)-[1,1′-biphenyl]-3-yl)-3-(((((S)-5-oxopyrrolidin-2-yl)methyl)amino)methyl)-4H-pyrido[1,2-a]pyrimidin-4-one;
8-(2,2′-dimethyl-3′-(4-oxo-3-(((((S)-5-oxopyrrolidin-2-yl)methyl)amino)methyl)-4H-pyrido[1,2-a]pyrimidin-8-yl)-[1,1′-biphenyl]-3-yl)-3-(((((R)-5-oxopyrrolidin-2-yl)methyl)amino)methyl)-4H-pyrido[1,2-a]pyrimidin-4-one;
8-(2,2′-dimethyl-3′-(4-oxo-3-(((((R)-5-oxopyrrolidin-2-yl)methyl)amino)methyl)-4H-pyrido[1,2-a]pyrimidin-8-yl)-[1,1′-biphenyl]-3-yl)-3-(((((R)-5-oxopyrrolidin-2-yl)methyl)amino)methyl)-4H-pyrido[1,2-a]pyrimidin-4-one;
8-(2,2′-dimethyl-3′-(4-oxo-3-(((((S)-5-oxopyrrolidin-2-yl)methyl)amino)methyl)-4H-pyrido[1,2-a]pyrimidin-8-yl)-[1,1′-biphenyl]-3-yl)-3-(((((S)-5-oxopyrrolidin-2-yl)methyl)amino)methyl)-4H-pyrido[1,2-a]pyrimidin-4-one;
N,N′-((((2,2′-dichloro-[1,1′-biphenyl]-3,3′-diyl)bis(3-methoxypyrazine-5,2-diyl))bis(azanediyl))bis(ethane-2,1-diyl))diacetamide;
N,N′-((((2,2′-dichloro-[1,1′-biphenyl]-3,3′-diyl)bis(6-methoxypyrazine-5,2-diyl))bis(azanediyl))bis(ethane-2,1-diyl))diacetamide;
8-(3′-((2R)-amino-2,3-dihydro-1H-inden-5-yl)-2′-chloro-2-methyl-[1,1′-biphenyl]-3-yl)-3-(((((R)-5-oxopyrrolidin-2-yl)methyl)amino)methyl)-4H-pyrido[1,2-a]pyrimidin-4-one;
8-(3′-((2R)-amino-2,3-dihydro-1H-inden-5-yl)-2′-chloro-2-methyl-[1,1′-biphenyl]-3-yl)-3-(((((S)-5-oxopyrrolidin-2-yl)methyl)amino)methyl)-4H-pyrido[1,2-a]pyrimidin-4-one;
8-(3′-((2S)-amino-2,3-dihydro-1H-inden-5-yl)-2′-chloro-2-methyl-[1,1′-biphenyl]-3-yl)-3-(((((R)-5-oxopyrrolidin-2-yl)methyl)amino)methyl)-4H-pyrido[1,2-a]pyrimidin-4-one;
8-(3′-((2S)-amino-2,3-dihydro-1H-inden-5-yl)-2′-chloro-2-methyl-[1,1′-biphenyl]-3-yl)-3-(((((S)-5-oxopyrrolidin-2-yl)methyl)amino)methyl)-4H-pyrido[1,2-a]pyrimidin-4-one;
8-(3′-((2R)-amino-2,3-dihydro-1H-inden-5-yl)-2,2′-dimethyl-[1,1′-biphenyl]-3-yl)-3-(((((R)-5-oxopyrrolidin-2-yl)methyl)amino)methyl)-4H-pyrido[1,2-a]pyrimidin-4-one;
8-(3′-((2R)-amino-2,3-dihydro-1H-inden-5-yl)-2,2′-dimethyl-[1,1′-biphenyl]-3-yl)-3-(((((S)-5-oxopyrrolidin-2-yl)methyl)amino)methyl)-4H-pyrido[1,2-a]pyrimidin-4-one;
8-(3′-((2S)-amino-2,3-dihydro-1H-inden-5-yl)-2,2′-dimethyl-[1,1′-biphenyl]-3-yl)-3-(((((R)-5-oxopyrrolidin-2-yl)methyl)amino)methyl)-4H-pyrido[1,2-a]pyrimidin-4-one;
8-(3′-((2S)-amino-2,3-dihydro-1H-inden-5-yl)-2,2′-dimethyl-[1,1′-biphenyl]-3-yl)-3-(((((S)-5-oxopyrrolidin-2-yl)methyl)amino)methyl)-4H-pyrido[1,2-a]pyrimidin-4-one;
(5S,5'S)-5,5′-((((4S,4'S)-(2,2′-dichloro-[1,1′-biphenyl]-3,3′-diyl)bis(1-methyl-1,2,3,4-tetrahydro-1,8-naphthyridine-7,4-diyl))bis(azanediyl))bis(methylene))bis(pyrrolidin-2-one);
(5S,5'S)-5,5′-((((4R,4'S)-(2,2′-dichloro-[1,1′-biphenyl]-3,3′-diyl)bis(1-methyl-1,2,3,4-tetrahydro-1,8-naphthyridine-7,4-diyl))bis(azanediyl))bis(methylene))bis(pyrrolidin-2-one);
(5S,5'S)-5,5′-((((4S,4′R)-(2,2′-dichloro-[1,1′-biphenyl]-3,3′-diyl)bis(1-methyl-1,2,3,4-tetrahydro-1,8-naphthyridine-7,4-diyl))bis(azanediyl))bis(methylene))bis(pyrrolidin-2-one);
(5S,5'S)-5,5′-((((4R,4′R)-(2,2′-dichloro-[1,1′-biphenyl]-3,3′-diyl)bis(1-methyl-1,2,3,4-tetrahydro-1,8-naphthyridine-7,4-diyl))bis(azanediyl))bis(methylene))bis(pyrrolidin-2-one);
(5R,5'S)-5,5′-((((4S,4'S)-(2,2′-dichloro-[1,1′-biphenyl]-3,3′-diyl)bis(1-methyl-1,2,3,4-tetrahydro-1,8-naphthyridine-7,4-diyl))bis(azanediyl))bis(methylene))bis(pyrrolidin-2-one);
(5R,5'S)-5,5′-((((4R,4'S)-(2,2′-dichloro-[1,1′-biphenyl]-3,3′-diyl)bis(1-methyl-1,2,3,4-tetrahydro-1,8-naphthyridine-7,4-diyl))bis(azanediyl))bis(methylene))bis(pyrrolidin-2-one);
(5R,5'S)-5,5′-((((4S,4′R)-(2,2′-dichloro-[1,1′-biphenyl]-3,3′-diyl)bis(1-methyl-1,2,3,4-tetrahydro-1,8-naphthyridine-7,4-diyl))bis(azanediyl))bis(methylene))bis(pyrrolidin-2-one);
(5R,5'S)-5,5′-((((4R,4′R)-(2,2′-dichloro-[1,1′-biphenyl]-3,3′-diyl)bis(1-methyl-1,2,3,4-tetrahydro-1,8-naphthyridine-7,4-diyl))bis(azanediyl))bis(methylene))bis(pyrrolidin-2-one);
(5S,5′R)-5,5′-((((4S,4'S)-(2,2′-dichloro-[1,1′-biphenyl]-3,3′-diyl)bis(1-methyl-1,2,3,4-tetrahydro-1,8-naphthyridine-7,4-diyl))bis(azanediyl))bis(methylene))bis(pyrrolidin-2-one);
(5S,5′R)-5,5′-((((4R,4'S)-(2,2′-dichloro-[1,1′-biphenyl]-3,3′-diyl)bis(1-methyl-1,2,3,4-tetrahydro-1,8-naphthyridine-7,4-diyl))bis(azanediyl))bis(methylene))bis(pyrrolidin-2-one);
(5S,5′R)-5,5′-((((4S,4′R)-(2,2′-dichloro-[1,1′-biphenyl]-3,3′-diyl)bis(1-methyl-1,2,3,4-tetrahydro-1,8-naphthyridine-7,4-diyl))bis(azanediyl))bis(methylene))bis(pyrrolidin-2-one);
(5S,5′R)-5,5′-((((4R,4′R)-(2,2′-dichloro-[1,1′-biphenyl]-3,3′-diyl)bis(1-methyl-1,2,3,4-tetrahydro-1,8-naphthyridine-7,4-diyl))bis(azanediyl))bis(methylene))bis(pyrrolidin-2-one);
(5R,5′R)-5,5′-((((4S,4'S)-(2,2′-dichloro-[1,1′-biphenyl]-3,3′-diyl)bis(1-methyl-1,2,3,4-tetrahydro-1,8-naphthyridine-7,4-diyl))bis(azanediyl))bis(methylene))bis(pyrrolidin-2-one);
(5R,5′R)-5,5′-((((4R,4'S)-(2,2′-dichloro-[1,1′-biphenyl]-3,3′-diyl)bis(1-methyl-1,2,3,4-tetrahydro-1,8-naphthyridine-7,4-diyl))bis(azanediyl))bis(methylene))bis(pyrrolidin-2-one);
(5R,5′R)-5,5′-((((4S,4′R)-(2,2′-dichloro-[1,1′-biphenyl]-3,3′-diyl)bis(1-methyl-1,2,3,4-tetrahydro-1,8-naphthyridine-7,4-diyl))bis(azanediyl))bis(methylene))bis(pyrrolidin-2-one);
(5R,5′R)-5,5′-((((4R,4′R)-(2,2′-dichloro-[1,1′-biphenyl]-3,3′-diyl)bis(1-methyl-1,2,3,4-tetrahydro-1,8-naphthyridine-7,4-diyl))bis(azanediyl))bis(methylene))bis(pyrrolidin-2-one);
2-amino-N-(5-(2-chloro-2′-methyl-3′-(4-oxo-3-(((((S)-5-oxopyrrolidin-2-yl)methyl)amino)methyl)-4H-pyrido[1,2-a]pyrimidin-8-yl)-[1,1′-biphenyl]-3-yl)-2,3-dihydro-1H-inden-(2S)-yl)acetamide;
2-amino-N-(5-(2-chloro-2′-methyl-3′-(4-oxo-3-(((((R)-5-oxopyrrolidin-2-yl)methyl)amino)methyl)-4H-pyrido[1,2-a]pyrimidin-8-yl)-[1,1′-biphenyl]-3-yl)-2,3-dihydro-1H-inden-(2S)-yl)acetamide;
2-amino-N-(5-(2-chloro-2′-methyl-3′-(4-oxo-3-(((((S)-5-oxopyrrolidin-2-yl)methyl)amino)methyl)-4H-pyrido[1,2-a]pyrimidin-8-yl)-[1,1′-biphenyl]-3-yl)-2,3-dihydro-1H-inden-(2R)-yl)acetamide;
2-amino-N-(5-(2-chloro-2′-methyl-3′-(4-oxo-3-(((((R)-5-oxopyrrolidin-2-yl)methyl)amino)methyl)-4H-pyrido[1,2-a]pyrimidin-8-yl)-[1,1′-biphenyl]-3-yl)-2,3-dihydro-1H-inden-(2R)-yl)acetamide;
(S)-5-((((S)-6-(2,2′-dichloro-3′-((S)-3-((((S)-5-oxopyrrolidin-2-yl)methyl)amino)-2,3-dihydrofuro[2,3-b]pyridin-6-yl)-[1,1′-biphenyl]-3-yl)-2,3-dihydrofuro[2,3-b]pyridin-3-yl)amino)methyl)pyrrolidin-2-one;
(S)-5-((((S)-6-(2,2′-dichloro-3′-((R)-3-((((S)-5-oxopyrrolidin-2-yl)methyl)amino)-2,3-dihydrofuro[2,3-b]pyridin-6-yl)-[1,1′-biphenyl]-3-yl)-2,3-dihydrofuro[2,3-b]pyridin-3-yl)amino)methyl)pyrrolidin-2-one;
(S)-5-((((S)-6-(2,2′-dichloro-3′-((S)-3-((((R)-5-oxopyrrolidin-2-yl)methyl)amino)-2,3-dihydrofuro[2,3-b]pyridin-6-yl)-[1,1′-biphenyl]-3-yl)-2,3-dihydrofuro[2,3-b]pyridin-3-yl)amino)methyl)pyrrolidin-2-one;
(S)-5-((((S)-6-(2,2′-dichloro-3′-((R)-3-((((R)-5-oxopyrrolidin-2-yl)methyl)amino)-2,3-dihydrofuro[2,3-b]pyridin-6-yl)-[1,1′-biphenyl]-3-yl)-2,3-dihydrofuro[2,3-b]pyridin-3-yl)amino)methyl)pyrrolidin-2-one;
(R)-5-((((S)-6-(2,2′-dichloro-3′-((S)-3-((((S)-5-oxopyrrolidin-2-yl)methyl)amino)-2,3-dihydrofuro[2,3-b]pyridin-6-yl)-[1,1′-biphenyl]-3-yl)-2,3-dihydrofuro[2,3-b]pyridin-3-yl)amino)methyl)pyrrolidin-2-one;
(R)-5-((((S)-6-(2,2′-dichloro-3′-((R)-3-((((S)-5-oxopyrrolidin-2-yl)methyl)amino)-2,3-dihydrofuro[2,3-b]pyridin-6-yl)-[1,1′-biphenyl]-3-yl)-2,3-dihydrofuro[2,3-b]pyridin-3-yl)amino)methyl)pyrrolidin-2-one;
(R)-5-((((S)-6-(2,2′-dichloro-3′-((S)-3-((((R)-5-oxopyrrolidin-2-yl)methyl)amino)-2,3-dihydrofuro[2,3-b]pyridin-6-yl)-[1,1′-biphenyl]-3-yl)-2,3-dihydrofuro[2,3-b]pyridin-3-yl)amino)methyl)pyrrolidin-2-one;
(R)-5-((((S)-6-(2,2′-dichloro-3′-((R)-3-((((R)-5-oxopyrrolidin-2-yl)methyl)amino)-2,3-dihydrofuro[2,3-b]pyridin-6-yl)-[1,1′-biphenyl]-3-yl)-2,3-dihydrofuro[2,3-b]pyridin-3-yl)amino)methyl)pyrrolidin-2-one;
(S)-5-((((R)-6-(2,2′-dichloro-3′-((S)-3-((((S)-5-oxopyrrolidin-2-yl)methyl)amino)-2,3-dihydrofuro[2,3-b]pyridin-6-yl)-[1,1′-biphenyl]-3-yl)-2,3-dihydrofuro[2,3-b]pyridin-3-yl)amino)methyl)pyrrolidin-2-one;
(S)-5-((((R)-6-(2,2′-dichloro-3′-((R)-3-((((S)-5-oxopyrrolidin-2-yl)methyl)amino)-2,3-dihydrofuro[2,3-b]pyridin-6-yl)-[1,1′-biphenyl]-3-yl)-2,3-dihydrofuro[2,3-b]pyridin-3-yl)amino)methyl)pyrrolidin-2-one;
(S)-5-((((R)-6-(2,2′-dichloro-3′-((S)-3-((((R)-5-oxopyrrolidin-2-yl)methyl)amino)-2,3-dihydrofuro[2,3-b]pyridin-6-yl)-[1,1′-biphenyl]-3-yl)-2,3-dihydrofuro[2,3-b]pyridin-3-yl)amino)methyl)pyrrolidin-2-one;
(S)-5-((((R)-6-(2,2′-dichloro-3′-((R)-3-((((R)-5-oxopyrrolidin-2-yl)methyl)amino)-2,3-dihydrofuro[2,3-b]pyridin-6-yl)-[1,1′-biphenyl]-3-yl)-2,3-dihydrofuro[2,3-b]pyridin-3-yl)amino)methyl)pyrrolidin-2-one;
(R)-5-((((R)-6-(2,2′-dichloro-3′-((S)-3-((((S)-5-oxopyrrolidin-2-yl)methyl)amino)-2,3-dihydrofuro[2,3-b]pyridin-6-yl)-[1,1′-biphenyl]-3-yl)-2,3-dihydrofuro[2,3-b]pyridin-3-yl)amino)methyl)pyrrolidin-2-one;
(R)-5-((((R)-6-(2,2′-dichloro-3′-((R)-3-((((S)-5-oxopyrrolidin-2-yl)methyl)amino)-2,3-dihydrofuro[2,3-b]pyridin-6-yl)-[1,1′-biphenyl]-3-yl)-2,3-dihydrofuro[2,3-b]pyridin-3-yl)amino)methyl)pyrrolidin-2-one;
(R)-5-((((R)-6-(2,2′-dichloro-3′-((S)-3-((((R)-5-oxopyrrolidin-2-yl)methyl)amino)-2,3-dihydrofuro[2,3-b]pyridin-6-yl)-[1,1′-biphenyl]-3-yl)-2,3-dihydrofuro[2,3-b]pyridin-3-yl)amino)methyl)pyrrolidin-2-one;
(R)-5-((((R)-6-(2,2′-dichloro-3′-((R)-3-((((R)-5-oxopyrrolidin-2-yl)methyl)amino)-2,3-dihydrofuro[2,3-b]pyridin-6-yl)-[1,1′-biphenyl]-3-yl)-2,3-dihydrofuro[2,3-b]pyridin-3-yl)amino)methyl)pyrrolidin-2-one;
(S)-2-amino-N—((S)-5-(2-chloro-2′-methyl-3′-(4-oxo-3-(((((S)-5-oxopyrrolidin-2-yl)methyl)amino)methyl)-4H-pyrido[1,2-a]pyrimidin-8-yl)-[1,1′-biphenyl]-3-yl)-2,3-dihydro-1H-inden-2-yl)-3-hydroxypropanamide;
(S)-2-amino-N—((R)-5-(2-chloro-2′-methyl-3′-(4-oxo-3-(((((S)-5-oxopyrrolidin-2-yl)methyl)amino)methyl)-4H-pyrido[1,2-a]pyrimidin-8-yl)-[1,1′-biphenyl]-3-yl)-2,3-dihydro-1H-inden-2-yl)-3-hydroxypropanamide;
(S)-2-amino-N—((S)-5-(2-chloro-2′-methyl-3′-(4-oxo-3-(((((R)-5-oxopyrrolidin-2-yl)methyl)amino)methyl)-4H-pyrido[1,2-a]pyrimidin-8-yl)-[1,1′-biphenyl]-3-yl)-2,3-dihydro-1H-inden-2-yl)-3-hydroxypropanamide;
(S)-2-amino-N—((R)-5-(2-chloro-2′-methyl-3′-(4-oxo-3-(((((R)-5-oxopyrrolidin-2-yl)methyl)amino)methyl)-4H-pyrido[1,2-a]pyrimidin-8-yl)-[1,1′-biphenyl]-3-yl)-2,3-dihydro-1H-inden-2-yl)-3-hydroxypropanamide;
(R)-2-amino-N—((S)-5-(2-chloro-2′-methyl-3′-(4-oxo-3-(((((S)-5-oxopyrrolidin-2-yl)methyl)amino)methyl)-4H-pyrido[1,2-a]pyrimidin-8-yl)-[1,1′-biphenyl]-3-yl)-2,3-dihydro-1H-inden-2-yl)-3-hydroxypropanamide;
(R)-2-amino-N—((R)-5-(2-chloro-2′-methyl-3′-(4-oxo-3-(((((S)-5-oxopyrrolidin-2-yl)methyl)amino)methyl)-4H-pyrido[1,2-a]pyrimidin-8-yl)-[1,1′-biphenyl]-3-yl)-2,3-dihydro-1H-inden-2-yl)-3-hydroxypropanamide;
(R)-2-amino-N—((S)-5-(2-chloro-2′-methyl-3′-(4-oxo-3-(((((R)-5-oxopyrrolidin-2-yl)methyl)amino)methyl)-4H-pyrido[1,2-a]pyrimidin-8-yl)-[1,1′-biphenyl]-3-yl)-2,3-dihydro-1H-inden-2-yl)-3-hydroxypropanamide;
(R)-2-amino-N—((R)-5-(2-chloro-2′-methyl-3′-(4-oxo-3-(((((R)-5-oxopyrrolidin-2-yl)methyl)amino)methyl)-4H-pyrido[1,2-a]pyrimidin-8-yl)-[1,1′-biphenyl]-3-yl)-2,3-dihydro-1H-inden-2-yl)-3-hydroxypropanamide;
(S)—N—((S)-5-(2-chloro-2′-methyl-3′-(4-oxo-3-(((((S)-5-oxopyrrolidin-2-yl)methyl)amino)methyl)-4H-pyrido[1,2-a]pyrimidin-8-yl)-[1,1′-biphenyl]-3-yl)-2,3-dihydro-1H-inden-2-yl)pyrrolidine-2-carboxamide;
(S)—N—((R)-5-(2-chloro-2′-methyl-3′-(4-oxo-3-(((((S)-5-oxopyrrolidin-2-yl)methyl)amino)methyl)-4H-pyrido[1,2-a]pyrimidin-8-yl)-[1,1′-biphenyl]-3-yl)-2,3-dihydro-1H-inden-2-yl)pyrrolidine-2-carboxamide;
(S)—N—((S)-5-(2-chloro-2′-methyl-3′-(4-oxo-3-(((((R)-5-oxopyrrolidin-2-yl)methyl)amino)methyl)-4H-pyrido[1,2-a]pyrimidin-8-yl)-[1,1′-biphenyl]-3-yl)-2,3-dihydro-1H-inden-2-yl)pyrrolidine-2-carboxamide;
(S)—N—((R)-5-(2-chloro-2′-methyl-3′-(4-oxo-3-(((((R)-5-oxopyrrolidin-2-yl)methyl)amino)methyl)-4H-pyrido[1,2-a]pyrimidin-8-yl)-[1,1′-biphenyl]-3-yl)-2,3-dihydro-1H-inden-2-yl)pyrrolidine-2-carboxamide;
(R)—N—((S)-5-(2-chloro-2′-methyl-3′-(4-oxo-3-(((((S)-5-oxopyrrolidin-2-yl)methyl)amino)methyl)-4H-pyrido[1,2-a]pyrimidin-8-yl)-[1,1′-biphenyl]-3-yl)-2,3-dihydro-1H-inden-2-yl)pyrrolidine-2-carboxamide;
(R)—N—((R)-5-(2-chloro-2′-methyl-3′-(4-oxo-3-(((((S)-5-oxopyrrolidin-2-yl)methyl)amino)methyl)-4H-pyrido[1,2-a]pyrimidin-8-yl)-[1,1′-biphenyl]-3-yl)-2,3-dihydro-1H-inden-2-yl)pyrrolidine-2-carboxamide;
(R)—N—((S)-5-(2-chloro-2′-methyl-3′-(4-oxo-3-(((((R)-5-oxopyrrolidin-2-yl)methyl)amino)methyl)-4H-pyrido[1,2-a]pyrimidin-8-yl)-[1,1′-biphenyl]-3-yl)-2,3-dihydro-1H-inden-2-yl)pyrrolidine-2-carboxamide;
(R)—N—((R)-5-(2-chloro-2′-methyl-3′-(4-oxo-3-(((((R)-5-oxopyrrolidin-2-yl)methyl)amino)methyl)-4H-pyrido[1,2-a]pyrimidin-8-yl)-[1,1′-biphenyl]-3-yl)-2,3-dihydro-1H-inden-2-yl)pyrrolidine-2-carboxamide;
8-(2,2′-dichloro-3′-(6-methoxy-5-(((((S)-5-oxopyrrolidin-2-yl)methyl)amino)methyl)pyrazin-2-yl)-[1,1′-biphenyl]-3-yl)-3-(((((S)-5-oxopyrrolidin-2-yl)methyl)amino)methyl)-4H-pyrido[1,2-a]pyrimidin-4-one;
8-(2,2′-dichloro-3′-(6-methoxy-5-(((((S)-5-oxopyrrolidin-2-yl)methyl)amino)methyl)pyrazin-2-yl)-[1,1′-biphenyl]-3-yl)-3-(((((R)-5-oxopyrrolidin-2-yl)methyl)amino)methyl)-4H-pyrido[1,2-a]pyrimidin-4-one;
8-(2,2′-dichloro-3′-(6-methoxy-5-(((((R)-5-oxopyrrolidin-2-yl)methyl)amino)methyl)pyrazin-2-yl)-[1,1′-biphenyl]-3-yl)-3-(((((S)-5-oxopyrrolidin-2-yl)methyl)amino)methyl)-4H-pyrido[1,2-a]pyrimidin-4-one;
8-(2,2′-dichloro-3′-(6-methoxy-5-(((((R)-5-oxopyrrolidin-2-yl)methyl)amino)methyl)pyrazin-2-yl)-[1,1′-biphenyl]-3-yl)-3-(((((R)-5-oxopyrrolidin-2-yl)methyl)amino)methyl)-4H-pyrido[1,2-a]pyrimidin-4-one;
8-(2,2′-dichloro-3′-(4-oxo-3-(((((S)-5-oxopyrrolidin-2-yl)methyl)amino)methyl)-4H-pyrido[1,2-a]pyrimidin-8-yl)-[1,1′-biphenyl]-3-yl)-3-(((((S)-5-oxopyrrolidin-2-yl)methyl)amino)methyl)-4H-pyrido[1,2-a]pyrimidin-4-one;
8-(2,2′-dichloro-3′-(4-oxo-3-(((((R)-5-oxopyrrolidin-2-yl)methyl)amino)methyl)-4H-pyrido[1,2-a]pyrimidin-8-yl)-[1,1′-biphenyl]-3-yl)-3-(((((S)-5-oxopyrrolidin-2-yl)methyl)amino)methyl)-4H-pyrido[1,2-a]pyrimidin-4-one;
8-(2,2′-dichloro-3′-(4-oxo-3-(((((S)-5-oxopyrrolidin-2-yl)methyl)amino)methyl)-4H-pyrido[1,2-a]pyrimidin-8-yl)-[1,1′-biphenyl]-3-yl)-3-(((((R)-5-oxopyrrolidin-2-yl)methyl)amino)methyl)-4H-pyrido[1,2-a]pyrimidin-4-one;
8-(2,2′-dichloro-3′-(4-oxo-3-(((((R)-5-oxopyrrolidin-2-yl)methyl)amino)methyl)-4H-pyrido[1,2-a]pyrimidin-8-yl)-[1,1′-biphenyl]-3-yl)-3-(((((R)-5-oxopyrrolidin-2-yl)methyl)amino)methyl)-4H-pyrido[1,2-a]pyrimidin-4-one;
8-(2′-chloro-3′-(6-methoxy-5-(((((S)-5-oxopyrrolidin-2-yl)methyl)amino)methyl)pyrazin-2-yl)-2-methyl-[1,1′-biphenyl]-3-yl)-3-(((((S)-5-oxopyrrolidin-2-yl)methyl)amino)methyl)-4H-quinolizin-4-one;
8-(2′-chloro-3′-(6-methoxy-5-(((((R)-5-oxopyrrolidin-2-yl)methyl)amino)methyl)pyrazin-2-yl)-2-methyl-[1,1′-biphenyl]-3-yl)-3-(((((S)-5-oxopyrrolidin-2-yl)methyl)amino)methyl)-4H-quinolizin-4-one;
8-(2′-chloro-3′-(6-methoxy-5-(((((S)-5-oxopyrrolidin-2-yl)methyl)amino)methyl)pyrazin-2-yl)-2-methyl-[1,1′-biphenyl]-3-yl)-3-(((((R)-5-oxopyrrolidin-2-yl)methyl)amino)methyl)-4H-quinolizin-4-one;
8-(2′-chloro-3′-(6-methoxy-5-(((((R)-5-oxopyrrolidin-2-yl)methyl)amino)methyl)pyrazin-2-yl)-2-methyl-[1,1′-biphenyl]-3-yl)-3-(((((R)-5-oxopyrrolidin-2-yl)methyl)amino)methyl)-4H-quinolizin-4-one;
8-(2′-chloro-3′-(6-chloro-5-(((((S)-5-oxopyrrolidin-2-yl)methyl)amino)methyl)pyridin-2-yl)-2-methyl-[1,1′-biphenyl]-3-yl)-3-(((((S)-5-oxopyrrolidin-2-yl)methyl)amino)methyl)-4H-pyrido[1,2-a]pyrimidin-4-one;
8-(2′-chloro-3′-(6-chloro-5-(((((S)-5-oxopyrrolidin-2-yl)methyl)amino)methyl)pyridin-2-yl)-2-methyl-[1,1′-biphenyl]-3-yl)-3-(((((R)-5-oxopyrrolidin-2-yl)methyl)amino)methyl)-4H-pyrido[1,2-a]pyrimidin-4-one;
8-(2′-chloro-3′-(6-chloro-5-(((((R)-5-oxopyrrolidin-2-yl)methyl)amino)methyl)pyridin-2-yl)-2-methyl-[1,1′-biphenyl]-3-yl)-3-(((((S)-5-oxopyrrolidin-2-yl)methyl)amino)methyl)-4H-pyrido[1,2-a]pyrimidin-4-one;
8-(2′-chloro-3′-(6-chloro-5-(((((R)-5-oxopyrrolidin-2-yl)methyl)amino)methyl)pyridin-2-yl)-2-methyl-[1,1′-biphenyl]-3-yl)-3-(((((R)-5-oxopyrrolidin-2-yl)methyl)amino)methyl)-4H-pyrido[1,2-a]pyrimidin-4-one;
(S)-5-((((6-(2,2′-dichloro-3′-((S)-8-methyl-5-((((S)-5-oxopyrrolidin-2-yl)methyl)amino)-5,6,7,8-tetrahydro-1,8-naphthyridin-2-yl)-[1,1′-biphenyl]-3-yl)-2-methoxypyridin-3-yl)methyl)amino)methyl)pyrrolidin-2-one;
(S)-5-((((6-(2,2′-dichloro-3′-((S)-8-methyl-5-((((R)-5-oxopyrrolidin-2-yl)methyl)amino)-5,6,7,8-tetrahydro-1,8-naphthyridin-2-yl)-[1,1′-biphenyl]-3-yl)-2-methoxypyridin-3-yl)methyl)amino)methyl)pyrrolidin-2-one;
(S)-5-((((6-(2,2′-dichloro-3′-((R)-8-methyl-5-((((S)-5-oxopyrrolidin-2-yl)methyl)amino)-5,6,7,8-tetrahydro-1,8-naphthyridin-2-yl)-[1,1′-biphenyl]-3-yl)-2-methoxypyridin-3-yl)methyl)amino)methyl)pyrrolidin-2-one;
(S)-5-((((6-(2,2′-dichloro-3′-((R)-8-methyl-5-((((R)-5-oxopyrrolidin-2-yl)methyl)amino)-5,6,7,8-tetrahydro-1,8-naphthyridin-2-yl)-[1,1′-biphenyl]-3-yl)-2-methoxypyridin-3-yl)methyl)amino)methyl)pyrrolidin-2-one;
(R)-5-((((6-(2,2′-dichloro-3′-((S)-8-methyl-5-((((S)-5-oxopyrrolidin-2-yl)methyl)amino)-5,6,7,8-tetrahydro-1,8-naphthyridin-2-yl)-[1,1′-biphenyl]-3-yl)-2-methoxypyridin-3-yl)methyl)amino)methyl)pyrrolidin-2-one;
(R)-5-((((6-(2,2′-dichloro-3′-((S)-8-methyl-5-((((R)-5-oxopyrrolidin-2-yl)methyl)amino)-5,6,7,8-tetrahydro-1,8-naphthyridin-2-yl)-[1,1′-biphenyl]-3-yl)-2-methoxypyridin-3-yl)methyl)amino)methyl)pyrrolidin-2-one;
(R)-5-((((6-(2,2′-dichloro-3′-((R)-8-methyl-5-((((S)-5-oxopyrrolidin-2-yl)methyl)amino)-5,6,7,8-tetrahydro-1,8-naphthyridin-2-yl)-[1,1′-biphenyl]-3-yl)-2-methoxypyridin-3-yl)methyl)amino)methyl)pyrrolidin-2-one;
(R)-5-((((6-(2,2′-dichloro-3′-((R)-8-methyl-5-((((R)-5-oxopyrrolidin-2-yl)methyl)amino)-5,6,7,8-tetrahydro-1,8-naphthyridin-2-yl)-[1,1′-biphenyl]-3-yl)-2-methoxypyridin-3-yl)methyl)amino)methyl)pyrrolidin-2-one;
8-(2′-chloro-3′-(6-methoxy-5-(((((S)-5-oxopyrrolidin-2-yl)methyl)amino)methyl)pyridin-2-yl)-2-methyl-[1,1′-biphenyl]-3-yl)-3-((methyl(((S)-5-oxopyrrolidin-2-yl)methyl)amino)methyl)-4H-pyrido[1,2-a]pyrimidin-4-one;
8-(2′-chloro-3′-(6-methoxy-5-(((((R)-5-oxopyrrolidin-2-yl)methyl)amino)methyl)pyridin-2-yl)-2-methyl-[1,1′-biphenyl]-3-yl)-3-((methyl(((S)-5-oxopyrrolidin-2-yl)methyl)amino)methyl)-4H-pyrido[1,2-a]pyrimidin-4-one;
8-(2′-chloro-3′-(6-methoxy-5-(((((S)-5-oxopyrrolidin-2-yl)methyl)amino)methyl)pyridin-2-yl)-2-methyl-[1,1′-biphenyl]-3-yl)-3-((methyl(((R)-5-oxopyrrolidin-2-yl)methyl)amino)methyl)-4H-pyrido[1,2-a]pyrimidin-4-one;
8-(2′-chloro-3′-(6-methoxy-5-(((((R)-5-oxopyrrolidin-2-yl)methyl)amino)methyl)pyridin-2-yl)-2-methyl-[1,1′-biphenyl]-3-yl)-3-((methyl(((R)-5-oxopyrrolidin-2-yl)methyl)amino)methyl)-4H-pyrido[1,2-a]pyrimidin-4-one;
(S)-8-(2′-chloro-3′-(5-((3-hydroxyazetidin-1-yl)methyl)-6-methoxypyridin-2-yl)-2-methyl-[1,1′-biphenyl]-3-yl)-3-((methyl((5-oxopyrrolidin-2-yl)methyl)amino)methyl)-4H-pyrido[1,2-a]pyrimidin-4-one;
(R)-8-(2′-chloro-3′-(5-((3-hydroxyazetidin-1-yl)methyl)-6-methoxypyridin-2-yl)-2-methyl-[1,1′-biphenyl]-3-yl)-3-((methyl((5-oxopyrrolidin-2-yl)methyl)amino)methyl)-4H-pyrido[1,2-a]pyrimidin-4-one;
(S)-5-((((5-(2,2′-dichloro-3′-((S)-8-methyl-5-((((S)-5-oxopyrrolidin-2-yl)methyl)amino)-5,6,7,8-tetrahydro-1,8-naphthyridin-2-yl)-[1,1′-biphenyl]-3-yl)-3-methoxypyrazin-2-yl)methyl)amino)methyl)pyrrolidin-2-one;
(S)-5-((((5-(2,2′-dichloro-3′-((R)-8-methyl-5-((((S)-5-oxopyrrolidin-2-yl)methyl)amino)-5,6,7,8-tetrahydro-1,8-naphthyridin-2-yl)-[1,1′-biphenyl]-3-yl)-3-methoxypyrazin-2-yl)methyl)amino)methyl)pyrrolidin-2-one;
(S)-5-((((5-(2,2′-dichloro-3′-((S)-8-methyl-5-((((R)-5-oxopyrrolidin-2-yl)methyl)amino)-5,6,7,8-tetrahydro-1,8-naphthyridin-2-yl)-[1,1′-biphenyl]-3-yl)-3-methoxypyrazin-2-yl)methyl)amino)methyl)pyrrolidin-2-one;
(S)-5-((((5-(2,2′-dichloro-3′-((R)-8-methyl-5-((((R)-5-oxopyrrolidin-2-yl)methyl)amino)-5,6,7,8-tetrahydro-1,8-naphthyridin-2-yl)-[1,1′-biphenyl]-3-yl)-3-methoxypyrazin-2-yl)methyl)amino)methyl)pyrrolidin-2-one;
(R)-5-((((5-(2,2′-dichloro-3′-((S)-8-methyl-5-((((S)-5-oxopyrrolidin-2-yl)methyl)amino)-5,6,7,8-tetrahydro-1,8-naphthyridin-2-yl)-[1,1′-biphenyl]-3-yl)-3-methoxypyrazin-2-yl)methyl)amino)methyl)pyrrolidin-2-one;
(R)-5-((((5-(2,2′-dichloro-3′-((R)-8-methyl-5-((((S)-5-oxopyrrolidin-2-yl)methyl)amino)-5,6,7,8-tetrahydro-1,8-naphthyridin-2-yl)-[1,1′-biphenyl]-3-yl)-3-methoxypyrazin-2-yl)methyl)amino)methyl)pyrrolidin-2-one;
(R)-5-((((5-(2,2′-dichloro-3′-((S)-8-methyl-5-((((R)-5-oxopyrrolidin-2-yl)methyl)amino)-5,6,7,8-tetrahydro-1,8-naphthyridin-2-yl)-[1,1′-biphenyl]-3-yl)-3-methoxypyrazin-2-yl)methyl)amino)methyl)pyrrolidin-2-one;
(R)-5-((((5-(2,2′-dichloro-3′-((R)-8-methyl-5-((((R)-5-oxopyrrolidin-2-yl)methyl)amino)-5,6,7,8-tetrahydro-1,8-naphthyridin-2-yl)-[1,1′-biphenyl]-3-yl)-3-methoxypyrazin-2-yl)methyl)amino)methyl)pyrrolidin-2-one;
8-(2′-chloro-3′-(6-fluoro-5-(((((S)-5-oxopyrrolidin-2-yl)methyl)amino)methyl)pyridin-2-yl)-2-methyl-[1,1′-biphenyl]-3-yl)-3-(((((S)-5-oxopyrrolidin-2-yl)methyl)amino)methyl)-4H-pyrido[1,2-a]pyrimidin-4-one;
8-(2′-chloro-3′-(6-fluoro-5-(((((R)-5-oxopyrrolidin-2-yl)methyl)amino)methyl)pyridin-2-yl)-2-methyl-[1,1′-biphenyl]-3-yl)-3-(((((S)-5-oxopyrrolidin-2-yl)methyl)amino)methyl)-4H-pyrido[1,2-a]pyrimidin-4-one;
8-(2′-chloro-3′-(6-fluoro-5-(((((S)-5-oxopyrrolidin-2-yl)methyl)amino)methyl)pyridin-2-yl)-2-methyl-[1,1′-biphenyl]-3-yl)-3-(((((R)-5-oxopyrrolidin-2-yl)methyl)amino)methyl)-4H-pyrido[1,2-a]pyrimidin-4-one;
8-(2′-chloro-3′-(6-fluoro-5-(((((R)-5-oxopyrrolidin-2-yl)methyl)amino)methyl)pyridin-2-yl)-2-methyl-[1,1′-biphenyl]-3-yl)-3-(((((R)-5-oxopyrrolidin-2-yl)methyl)amino)methyl)-4H-pyrido[1,2-a]pyrimidin-4-one;
2-(2,2′-dichloro-3′-(6-methoxy-5-(((((S)-5-oxopyrrolidin-2-yl)methyl)amino)methyl)pyrazin-2-yl)-[1,1′-biphenyl]-3-yl)-7-(((((S)-5-oxopyrrolidin-2-yl)methyl)amino)methyl)-4H-pyrido[1,2-a]pyrimidin-4-one;
2-(2,2′-dichloro-3′-(6-methoxy-5-(((((R)-5-oxopyrrolidin-2-yl)methyl)amino)methyl)pyrazin-2-yl)-[1,1′-biphenyl]-3-yl)-7-(((((S)-5-oxopyrrolidin-2-yl)methyl)amino)methyl)-4H-pyrido[1,2-a]pyrimidin-4-one;
2-(2,2′-dichloro-3′-(6-methoxy-5-(((((S)-5-oxopyrrolidin-2-yl)methyl)amino)methyl)pyrazin-2-yl)-[1,1′-biphenyl]-3-yl)-7-(((((R)-5-oxopyrrolidin-2-yl)methyl)amino)methyl)-4H-pyrido[1,2-a]pyrimidin-4-one;
2-(2,2′-dichloro-3′-(6-methoxy-5-(((((R)-5-oxopyrrolidin-2-yl)methyl)amino)methyl)pyrazin-2-yl)-[1,1′-biphenyl]-3-yl)-7-(((((R)-5-oxopyrrolidin-2-yl)methyl)amino)methyl)-4H-pyrido[1,2-a]pyrimidin-4-one;
2-(2,2′-dichloro-3′-(6-methoxy-5-(((((S)-5-oxopyrrolidin-2-yl)methyl)amino)methyl)pyridin-2-yl)-[1,1′-biphenyl]-3-yl)-7-(((((S)-5-oxopyrrolidin-2-yl)methyl)amino)methyl)-4H-pyrido[1,2-a]pyrimidin-4-one;
2-(2,2′-dichloro-3′-(6-methoxy-5-(((((R)-5-oxopyrrolidin-2-yl)methyl)amino)methyl)pyridin-2-yl)-[1,1′-biphenyl]-3-yl)-7-(((((S)-5-oxopyrrolidin-2-yl)methyl)amino)methyl)-4H-pyrido[1,2-a]pyrimidin-4-one;
2-(2,2′-dichloro-3′-(6-methoxy-5-(((((S)-5-oxopyrrolidin-2-yl)methyl)amino)methyl)pyridin-2-yl)-[1,1′-biphenyl]-3-yl)-7-(((((R)-5-oxopyrrolidin-2-yl)methyl)amino)methyl)-4H-pyrido[1,2-a]pyrimidin-4-one;
2-(2,2′-dichloro-3′-(6-methoxy-5-(((((R)-5-oxopyrrolidin-2-yl)methyl)amino)methyl)pyridin-2-yl)-[1,1′-biphenyl]-3-yl)-7-(((((R)-5-oxopyrrolidin-2-yl)methyl)amino)methyl)-4H-pyrido[1,2-a]pyrimidin-4-one;
2-(2,2′-dichloro-3′-(4-oxo-7-(((((S)-5-oxopyrrolidin-2-yl)methyl)amino)methyl)-4H-pyrido[1,2-a]pyrimidin-2-yl)-[1,1′-biphenyl]-3-yl)-7-(((((S)-5-oxopyrrolidin-2-yl)methyl)amino)methyl)-4H-pyrido[1,2-a]pyrimidin-4-one;
2-(2,2′-dichloro-3′-(4-oxo-7-(((((S)-5-oxopyrrolidin-2-yl)methyl)amino)methyl)-4H-pyrido[1,2-a]pyrimidin-2-yl)-[1,1′-biphenyl]-3-yl)-7-(((((R)-5-oxopyrrolidin-2-yl)methyl)amino)methyl)-4H-pyrido[1,2-a]pyrimidin-4-one;
2-(2,2′-dichloro-3′-(4-oxo-7-(((((R)-5-oxopyrrolidin-2-yl)methyl)amino)methyl)-4H-pyrido[1,2-a]pyrimidin-2-yl)-[1,1′-biphenyl]-3-yl)-7-(((((S)-5-oxopyrrolidin-2-yl)methyl)amino)methyl)-4H-pyrido[1,2-a]pyrimidin-4-one;
2-(2,2′-dichloro-3′-(4-oxo-7-(((((R)-5-oxopyrrolidin-2-yl)methyl)amino)methyl)-4H-pyrido[1,2-a]pyrimidin-2-yl)-[1,1′-biphenyl]-3-yl)-7-(((((R)-5-oxopyrrolidin-2-yl)methyl)amino)methyl)-4H-pyrido[1,2-a]pyrimidin-4-one;
8-(2′-chloro-2-methyl-3′-(5-(((((S)-5-oxopyrrolidin-2-yl)methyl)amino)methyl)pyridin-2-yl)-[1,1′-biphenyl]-3-yl)-3-(((((S)-5-oxopyrrolidin-2-yl)methyl)amino)methyl)-4H-pyrido[1,2-a]pyrimidin-4-one;
8-(2′-chloro-2-methyl-3′-(5-(((((R)-5-oxopyrrolidin-2-yl)methyl)amino)methyl)pyridin-2-yl)-[1,1′-biphenyl]-3-yl)-3-(((((S)-5-oxopyrrolidin-2-yl)methyl)amino)methyl)-4H-pyrido[1,2-a]pyrimidin-4-one;
8-(2′-chloro-2-methyl-3′-(5-(((((S)-5-oxopyrrolidin-2-yl)methyl)amino)methyl)pyridin-2-yl)-[1,1′-biphenyl]-3-yl)-3-(((((R)-5-oxopyrrolidin-2-yl)methyl)amino)methyl)-4H-pyrido[1,2-a]pyrimidin-4-one;
8-(2′-chloro-2-methyl-3′-(5-(((((R)-5-oxopyrrolidin-2-yl)methyl)amino)methyl)pyridin-2-yl)-[1,1′-biphenyl]-3-yl)-3-(((((R)-5-oxopyrrolidin-2-yl)methyl)amino)methyl)-4H-pyrido[1,2-a]pyrimidin-4-one;
8-(2′-chloro-3′-(6-fluoro-5-(((((S)-5-oxopyrrolidin-2-yl)methyl)amino)methyl)pyridin-2-yl)-2-methyl-[1,1′-biphenyl]-3-yl)-3-((((S)-5-oxopyrrolidin-3-yl)amino)methyl)-4H-pyrido[1,2-a]pyrimidin-4-one;
8-(2′-chloro-3′-(6-fluoro-5-(((((R)-5-oxopyrrolidin-2-yl)methyl)amino)methyl)pyridin-2-yl)-2-methyl-[1,1′-biphenyl]-3-yl)-3-((((S)-5-oxopyrrolidin-3-yl)amino)methyl)-4H-pyrido[1,2-a]pyrimidin-4-one;
8-(2′-chloro-3′-(6-fluoro-5-(((((S)-5-oxopyrrolidin-2-yl)methyl)amino)methyl)pyridin-2-yl)-2-methyl-[1,1′-biphenyl]-3-yl)-3-((((R)-5-oxopyrrolidin-3-yl)amino)methyl)-4H-pyrido[1,2-a]pyrimidin-4-one;
8-(2′-chloro-3′-(6-fluoro-5-(((((R)-5-oxopyrrolidin-2-yl)methyl)amino)methyl)pyridin-2-yl)-2-methyl-[1,1′-biphenyl]-3-yl)-3-((((R)-5-oxopyrrolidin-3-yl)amino)methyl)-4H-pyrido[1,2-a]pyrimidin-4-one;
8-(2,2′-dichloro-3′-(6-methoxy-5-((((R)-5-oxopyrrolidin-3-yl)amino)methyl)pyridin-2-yl)-[1,1′-biphenyl]-3-yl)-3-((((R)-5-oxopyrrolidin-3-yl)amino)methyl)-4H-pyrido[1,2-a]pyrimidin-4-one;
8-(2,2′-dichloro-3′-(6-methoxy-5-((((R)-5-oxopyrrolidin-3-yl)amino)methyl)pyridin-2-yl)-[1,1′-biphenyl]-3-yl)-3-((((S)-5-oxopyrrolidin-3-yl)amino)methyl)-4H-pyrido[1,2-a]pyrimidin-4-one;
8-(2,2′-dichloro-3′-(6-methoxy-5-((((S)-5-oxopyrrolidin-3-yl)amino)methyl)pyridin-2-yl)-[1,1′-biphenyl]-3-yl)-3-((((R)-5-oxopyrrolidin-3-yl)amino)methyl)-4H-pyrido[1,2-a]pyrimidin-4-one;
8-(2,2′-dichloro-3′-(6-methoxy-5-((((S)-5-oxopyrrolidin-3-yl)amino)methyl)pyridin-2-yl)-[1,1′-biphenyl]-3-yl)-3-((((S)-5-oxopyrrolidin-3-yl)amino)methyl)-4H-pyrido[1,2-a]pyrimidin-4-one;
8-(2′-chloro-3′-(6-(dimethylamino)-5-(((((S)-5-oxopyrrolidin-2-yl)methyl)amino)methyl)pyridin-2-yl)-2-methyl-[1,1′-biphenyl]-3-yl)-3-(((((S)-5-oxopyrrolidin-2-yl)methyl)amino)methyl)-4H-pyrido[1,2-a]pyrimidin-4-one;
8-(2′-chloro-3′-(6-(dimethylamino)-5-(((((R)-5-oxopyrrolidin-2-yl)methyl)amino)methyl)pyridin-2-yl)-2-methyl-[1,1′-biphenyl]-3-yl)-3-(((((S)-5-oxopyrrolidin-2-yl)methyl)amino)methyl)-4H-pyrido[1,2-a]pyrimidin-4-one;
8-(2′-chloro-3′-(6-(dimethylamino)-5-(((((S)-5-oxopyrrolidin-2-yl)methyl)amino)methyl)pyridin-2-yl)-2-methyl-[1,1′-biphenyl]-3-yl)-3-(((((R)-5-oxopyrrolidin-2-yl)methyl)amino)methyl)-4H-pyrido[1,2-a]pyrimidin-4-one;
8-(2′-chloro-3′-(6-(dimethylamino)-5-(((((R)-5-oxopyrrolidin-2-yl)methyl)amino)methyl)pyridin-2-yl)-2-methyl-[1,1′-biphenyl]-3-yl)-3-(((((R)-5-oxopyrrolidin-2-yl)methyl)amino)methyl)-4H-pyrido[1,2-a]pyrimidin-4-one;
8-(2′-chloro-2-methyl-4″-(((((S)-5-oxopyrrolidin-2-yl)methyl)amino)methyl)-[1,1′:3′,1″-terphenyl]-3-yl)-3-(((((S)-5-oxopyrrolidin-2-yl)methyl)amino)methyl)-4H-pyrido[1,2-a]pyrimidin-4-one;
8-(2′-chloro-2-methyl-4″-(((((R)-5-oxopyrrolidin-2-yl)methyl)amino)methyl)-[1,1′:3′,1″-terphenyl]-3-yl)-3-(((((S)-5-oxopyrrolidin-2-yl)methyl)amino)methyl)-4H-pyrido[1,2-a]pyrimidin-4-one;
8-(2′-chloro-2-methyl-4″-(((((S)-5-oxopyrrolidin-2-yl)methyl)amino)methyl)-[1,1′:3′,1″-terphenyl]-3-yl)-3-(((((R)-5-oxopyrrolidin-2-yl)methyl)amino)methyl)-4H-pyrido[1,2-a]pyrimidin-4-one;
8-(2′-chloro-2-methyl-4″-(((((R)-5-oxopyrrolidin-2-yl)methyl)amino)methyl)-[1,1′:3′,1″-terphenyl]-3-yl)-3-(((((R)-5-oxopyrrolidin-2-yl)methyl)amino)methyl)-4H-pyrido[1,2-a]pyrimidin-4-one;
8-(2′-chloro-3′-methoxy-2-methyl-4″-(((((S)-5-oxopyrrolidin-2-yl)methyl)amino)methyl)-[1,1′:3′,1″-terphenyl]-3-yl)-3-(((((S)-5-oxopyrrolidin-2-yl)methyl)amino)methyl)-4H-pyrido[1,2-a]pyrimidin-4-one;
8-(2′-chloro-3′-methoxy-2-methyl-4″-(((((R)-5-oxopyrrolidin-2-yl)methyl)amino)methyl)-[1,1′:3′,1″-terphenyl]-3-yl)-3-(((((S)-5-oxopyrrolidin-2-yl)methyl)amino)methyl)-4H-pyrido[1,2-a]pyrimidin-4-one;
8-(2′-chloro-3′-methoxy-2-methyl-4″-(((((S)-5-oxopyrrolidin-2-yl)methyl)amino)methyl)-[1,1′:3′,1″-terphenyl]-3-yl)-3-(((((R)-5-oxopyrrolidin-2-yl)methyl)amino)methyl)-4H-pyrido[1,2-a]pyrimidin-4-one;
8-(2′-chloro-3′-methoxy-2-methyl-4″-(((((R)-5-oxopyrrolidin-2-yl)methyl)amino)methyl)-[1,1′:3′,1″-terphenyl]-3-yl)-3-(((((R)-5-oxopyrrolidin-2-yl)methyl)amino)methyl)-4H-pyrido[1,2-a]pyrimidin-4-one;
8-(2′,3″-dichloro-2-methyl-4″-(((((S)-5-oxopyrrolidin-2-yl)methyl)amino)methyl)-[1,1′:3′,1″-terphenyl]-3-yl)-3-(((((S)-5-oxopyrrolidin-2-yl)methyl)amino)methyl)-4H-pyrido[1,2-a]pyrimidin-4-one;
8-(2′,3″-dichloro-2-methyl-4″-(((((R)-5-oxopyrrolidin-2-yl)methyl)amino)methyl)-[1,1′:3′,1″-terphenyl]-3-yl)-3-(((((S)-5-oxopyrrolidin-2-yl)methyl)amino)methyl)-4H-pyrido[1,2-a]pyrimidin-4-one;
8-(2′,3″-dichloro-2-methyl-4″-(((((S)-5-oxopyrrolidin-2-yl)methyl)amino)methyl)-[1,1′:3′,1″-terphenyl]-3-yl)-3-(((((R)-5-oxopyrrolidin-2-yl)methyl)amino)methyl)-4H-pyrido[1,2-a]pyrimidin-4-one;
8-(2′,3″-dichloro-2-methyl-4″-(((((R)-5-oxopyrrolidin-2-yl)methyl)amino)methyl)-[1,1′:3′,1″-terphenyl]-3-yl)-3-(((((R)-5-oxopyrrolidin-2-yl)methyl)amino)methyl)-4H-pyrido[1,2-a]pyrimidin-4-one;
8-(2′-chloro-2-methyl-3′-((R)-8-methyl-5-((((R)-5-oxopyrrolidin-2-yl)methyl)amino)-5,6,7,8-tetrahydro-1,8-naphthyridin-2-yl)-[1,1′-biphenyl]-3-yl)-3-(((((R)-5-oxopyrrolidin-2-yl)methyl)amino)methyl)-4H-pyrido[1,2-a]pyrimidin-4-one;
8-(2′-chloro-2-methyl-3′-((R)-8-methyl-5-((((R)-5-oxopyrrolidin-2-yl)methyl)amino)-5,6,7,8-tetrahydro-1,8-naphthyridin-2-yl)-[1,1′-biphenyl]-3-yl)-3-(((((S)-5-oxopyrrolidin-2-yl)methyl)amino)methyl)-4H-pyrido[1,2-a]pyrimidin-4-one;
8-(2′-chloro-2-methyl-3′-((R)-8-methyl-5-((((S)-5-oxopyrrolidin-2-yl)methyl)amino)-5,6,7,8-tetrahydro-1,8-naphthyridin-2-yl)-[1,1′-biphenyl]-3-yl)-3-(((((R)-5-oxopyrrolidin-2-yl)methyl)amino)methyl)-4H-pyrido[1,2-a]pyrimidin-4-one;
8-(2′-chloro-2-methyl-3′-((R)-8-methyl-5-((((S)-5-oxopyrrolidin-2-yl)methyl)amino)-5,6,7,8-tetrahydro-1,8-naphthyridin-2-yl)-[1,1′-biphenyl]-3-yl)-3-(((((S)-5-oxopyrrolidin-2-yl)methyl)amino)methyl)-4H-pyrido[1,2-a]pyrimidin-4-one;
8-(2′-chloro-2-methyl-3′-((S)-8-methyl-5-((((R)-5-oxopyrrolidin-2-yl)methyl)amino)-5,6,7,8-tetrahydro-1,8-naphthyridin-2-yl)-[1,1′-biphenyl]-3-yl)-3-(((((R)-5-oxopyrrolidin-2-yl)methyl)amino)methyl)-4H-pyrido[1,2-a]pyrimidin-4-one;
8-(2′-chloro-2-methyl-3′-((S)-8-methyl-5-((((R)-5-oxopyrrolidin-2-yl)methyl)amino)-5,6,7,8-tetrahydro-1,8-naphthyridin-2-yl)-[1,1′-biphenyl]-3-yl)-3-(((((S)-5-oxopyrrolidin-2-yl)methyl)amino)methyl)-4H-pyrido[1,2-a]pyrimidin-4-one;
8-(2′-chloro-2-methyl-3′-((S)-8-methyl-5-((((S)-5-oxopyrrolidin-2-yl)methyl)amino)-5,6,7,8-tetrahydro-1,8-naphthyridin-2-yl)-[1,1′-biphenyl]-3-yl)-3-(((((R)-5-oxopyrrolidin-2-yl)methyl)amino)methyl)-4H-pyrido[1,2-a]pyrimidin-4-one;
8-(2′-chloro-2-methyl-3′-((S)-8-methyl-5-((((S)-5-oxopyrrolidin-2-yl)methyl)amino)-5,6,7,8-tetrahydro-1,8-naphthyridin-2-yl)-[1,1′-biphenyl]-3-yl)-3-(((((S)-5-oxopyrrolidin-2-yl)methyl)amino)methyl)-4H-pyrido[1,2-a]pyrimidin-4-one;
N-(2-(((6-(3′-(3-(((2-acetamidoethyl)amino)methyl)-4-oxo-4H-pyrido[1,2-a]pyrimidin-8-yl)-2,2′-dichloro-[1,1′-biphenyl]-3-yl)-2-methoxypyridin-3-yl)methyl)amino)ethyl)acetamide;
8-(2,2′-dichloro-3′-(6-methoxy-5-((7-oxo-2,6-diazaspiro[3.4]octan-2-yl)methyl)pyridin-2-yl)-[1,1′-biphenyl]-3-yl)-3-((7-oxo-2,6-diazaspiro[3.4]octan-2-yl)methyl)-4H-pyrido[1,2-a]pyrimidin-4-one;
(S)-8-(3′-(5-(3-aminoazetidin-1-yl)-6-methoxypyrazin-2-yl)-2,2′-dichloro-[1,1′-biphenyl]-3-yl)-3-((((5-oxopyrrolidin-2-yl)methyl)amino)methyl)-4H-pyrido[1,2-a]pyrimidin-4-one;
(R)-8-(3′-(5-(3-aminoazetidin-1-yl)-6-methoxypyrazin-2-yl)-2,2′-dichloro-[1,1′-biphenyl]-3-yl)-3-((((5-oxopyrrolidin-2-yl)methyl)amino)methyl)-4H-pyrido[1,2-a]pyrimidin-4-one;
8-(2′-chloro-3′-(6-ethoxy-5-(((((S)-5-oxopyrrolidin-2-yl)methyl)amino)methyl)pyridin-2-yl)-2-methyl-[1,1′-biphenyl]-3-yl)-3-(((((S)-5-oxopyrrolidin-2-yl)methyl)amino)methyl)-4H-pyrido[1,2-a]pyrimidin-4-one;
8-(2′-chloro-3′-(6-ethoxy-5-(((((R)-5-oxopyrrolidin-2-yl)methyl)amino)methyl)pyridin-2-yl)-2-methyl-[1,1′-biphenyl]-3-yl)-3-(((((S)-5-oxopyrrolidin-2-yl)methyl)amino)methyl)-4H-pyrido[1,2-a]pyrimidin-4-one;
8-(2′-chloro-3′-(6-ethoxy-5-(((((S)-5-oxopyrrolidin-2-yl)methyl)amino)methyl)pyridin-2-yl)-2-methyl-[1,1′-biphenyl]-3-yl)-3-(((((R)-5-oxopyrrolidin-2-yl)methyl)amino)methyl)-4H-pyrido[1,2-a]pyrimidin-4-one;
8-(2′-chloro-3′-(6-ethoxy-5-(((((R)-5-oxopyrrolidin-2-yl)methyl)amino)methyl)pyridin-2-yl)-2-methyl-[1,1′-biphenyl]-3-yl)-3-(((((R)-5-oxopyrrolidin-2-yl)methyl)amino)methyl)-4H-pyrido[1,2-a]pyrimidin-4-one;
8-(2′-chloro-2-methyl-3′-(6-(methylamino)-5-(((((S)-5-oxopyrrolidin-2-yl)methyl)amino)methyl)pyridin-2-yl)-[1,1′-biphenyl]-3-yl)-3-(((((S)-5-oxopyrrolidin-2-yl)methyl)amino)methyl)-4H-pyrido[1,2-a]pyrimidin-4-one;
8-(2′-chloro-2-methyl-3′-(6-(methylamino)-5-(((((R)-5-oxopyrrolidin-2-yl)methyl)amino)methyl)pyridin-2-yl)-[1,1′-biphenyl]-3-yl)-3-(((((S)-5-oxopyrrolidin-2-yl)methyl)amino)methyl)-4H-pyrido[1,2-a]pyrimidin-4-one;
8-(2′-chloro-2-methyl-3′-(6-(methylamino)-5-(((((S)-5-oxopyrrolidin-2-yl)methyl)amino)methyl)pyridin-2-yl)-[1,1′-biphenyl]-3-yl)-3-(((((R)-5-oxopyrrolidin-2-yl)methyl)amino)methyl)-4H-pyrido[1,2-a]pyrimidin-4-one;
8-(2′-chloro-2-methyl-3′-(6-(methylamino)-5-(((((R)-5-oxopyrrolidin-2-yl)methyl)amino)methyl)pyridin-2-yl)-[1,1′-biphenyl]-3-yl)-3-(((((R)-5-oxopyrrolidin-2-yl)methyl)amino)methyl)-4H-pyrido[1,2-a]pyrimidin-4-one;
8-(2′-chloro-3′-(6-(ethylamino)-5-(((((S)-5-oxopyrrolidin-2-yl)methyl)amino)methyl)pyridin-2-yl)-2-methyl-[1,1′-biphenyl]-3-yl)-3-(((((S)-5-oxopyrrolidin-2-yl)methyl)amino)methyl)-4H-pyrido[1,2-a]pyrimidin-4-one;
8-(2′-chloro-3′-(6-(ethylamino)-5-(((((R)-5-oxopyrrolidin-2-yl)methyl)amino)methyl)pyridin-2-yl)-2-methyl-[1,1′-biphenyl]-3-yl)-3-(((((S)-5-oxopyrrolidin-2-yl)methyl)amino)methyl)-4H-pyrido[1,2-a]pyrimidin-4-one;
8-(2′-chloro-3′-(6-(ethylamino)-5-(((((S)-5-oxopyrrolidin-2-yl)methyl)amino)methyl)pyridin-2-yl)-2-methyl-[1,1′-biphenyl]-3-yl)-3-(((((R)-5-oxopyrrolidin-2-yl)methyl)amino)methyl)-4H-pyrido[1,2-a]pyrimidin-4-one;
8-(2′-chloro-3′-(6-(ethylamino)-5-(((((R)-5-oxopyrrolidin-2-yl)methyl)amino)methyl)pyridin-2-yl)-2-methyl-[1,1′-biphenyl]-3-yl)-3-(((((R)-5-oxopyrrolidin-2-yl)methyl)amino)methyl)-4H-pyrido[1,2-a]pyrimidin-4-one;
8-(2,2′-dichloro-3′-(5-(((2-hydroxyethyl)amino)methyl)-6-methoxypyridin-2-yl)-[1,1′-biphenyl]-3-yl)-3-(((2-hydroxyethyl)amino)methyl)-4H-pyrido[1,2-a]pyrimidin-4-one;
8-(2′-chloro-2-methyl-3′-((R)-1-((((S)-5-oxopyrrolidin-2-yl)methyl)amino)-2,3-dihydro-1H-inden-5-yl)-[1,1′-biphenyl]-3-yl)-3-(((((S)-5-oxopyrrolidin-2-yl)methyl)amino)methyl)-4H-pyrido[1,2-a]pyrimidin-4-one;
8-(2′-chloro-2-methyl-3′-((R)-1-((((R)-5-oxopyrrolidin-2-yl)methyl)amino)-2,3-dihydro-1H-inden-5-yl)-[1,1′-biphenyl]-3-yl)-3-(((((S)-5-oxopyrrolidin-2-yl)methyl)amino)methyl)-4H-pyrido[1,2-a]pyrimidin-4-one;
8-(2′-chloro-2-methyl-3′-((R)-1-((((S)-5-oxopyrrolidin-2-yl)methyl)amino)-2,3-dihydro-1H-inden-5-yl)-[1,1′-biphenyl]-3-yl)-3-(((((R)-5-oxopyrrolidin-2-yl)methyl)amino)methyl)-4H-pyrido[1,2-a]pyrimidin-4-one;
8-(2′-chloro-2-methyl-3′-((R)-1-((((R)-5-oxopyrrolidin-2-yl)methyl)amino)-2,3-dihydro-1H-inden-5-yl)-[1,1′-biphenyl]-3-yl)-3-(((((R)-5-oxopyrrolidin-2-yl)methyl)amino)methyl)-4H-pyrido[1,2-a]pyrimidin-4-one;
8-(2′-chloro-2-methyl-3′-((S)-1-((((S)-5-oxopyrrolidin-2-yl)methyl)amino)-2,3-dihydro-1H-inden-5-yl)-[1,1′-biphenyl]-3-yl)-3-(((((S)-5-oxopyrrolidin-2-yl)methyl)amino)methyl)-4H-pyrido[1,2-a]pyrimidin-4-one;
8-(2′-chloro-2-methyl-3′-((S)-1-((((R)-5-oxopyrrolidin-2-yl)methyl)amino)-2,3-dihydro-1H-inden-5-yl)-[1,1′-biphenyl]-3-yl)-3-(((((S)-5-oxopyrrolidin-2-yl)methyl)amino)methyl)-4H-pyrido[1,2-a]pyrimidin-4-one;
8-(2′-chloro-2-methyl-3′-((S)-1-((((S)-5-oxopyrrolidin-2-yl)methyl)amino)-2,3-dihydro-1H-inden-5-yl)-[1,1′-biphenyl]-3-yl)-3-(((((R)-5-oxopyrrolidin-2-yl)methyl)amino)methyl)-4H-pyrido[1,2-a]pyrimidin-4-one;
8-(2′-chloro-2-methyl-3′-((S)-1-((((R)-5-oxopyrrolidin-2-yl)methyl)amino)-2,3-dihydro-1H-inden-5-yl)-[1,1′-biphenyl]-3-yl)-3-(((((R)-5-oxopyrrolidin-2-yl)methyl)amino)methyl)-4H-pyrido[1,2-a]pyrimidin-4-one;
8-(2′-chloro-3′-(4-methoxy-5-(((((S)-5-oxopyrrolidin-2-yl)methyl)amino)methyl)pyridin-2-yl)-2-methyl-[1,1′-biphenyl]-3-yl)-3-(((((S)-5-oxopyrrolidin-2-yl)methyl)amino)methyl)-4H-pyrido[1,2-a]pyrimidin-4-one;
8-(2′-chloro-3′-(4-methoxy-5-(((((R)-5-oxopyrrolidin-2-yl)methyl)amino)methyl)pyridin-2-yl)-2-methyl-[1,1′-biphenyl]-3-yl)-3-(((((S)-5-oxopyrrolidin-2-yl)methyl)amino)methyl)-4H-pyrido[1,2-a]pyrimidin-4-one;
8-(2′-chloro-3′-(4-methoxy-5-(((((S)-5-oxopyrrolidin-2-yl)methyl)amino)methyl)pyridin-2-yl)-2-methyl-[1,1′-biphenyl]-3-yl)-3-(((((R)-5-oxopyrrolidin-2-yl)methyl)amino)methyl)-4H-pyrido[1,2-a]pyrimidin-4-one;
8-(2′-chloro-3′-(4-methoxy-5-(((((R)-5-oxopyrrolidin-2-yl)methyl)amino)methyl)pyridin-2-yl)-2-methyl-[1,1′-biphenyl]-3-yl)-3-(((((R)-5-oxopyrrolidin-2-yl)methyl)amino)methyl)-4H-pyrido[1,2-a]pyrimidin-4-one;
8-(2′-chloro-2-methyl-3′-(5-(((((S)-5-oxopyrrolidin-2-yl)methyl)amino)methyl)-6-(trifluoromethyl)pyridin-2-yl)-[1,1′-biphenyl]-3-yl)-3-(((((S)-5-oxopyrrolidin-2-yl)methyl)amino)methyl)-4H-pyrido[1,2-a]pyrimidin-4-one;
8-(2′-chloro-2-methyl-3′-(5-(((((R)-5-oxopyrrolidin-2-yl)methyl)amino)methyl)-6-(trifluoromethyl)pyridin-2-yl)-[1,1′-biphenyl]-3-yl)-3-(((((S)-5-oxopyrrolidin-2-yl)methyl)amino)methyl)-4H-pyrido[1,2-a]pyrimidin-4-one;
8-(2′-chloro-2-methyl-3′-(5-(((((S)-5-oxopyrrolidin-2-yl)methyl)amino)methyl)-6-(trifluoromethyl)pyridin-2-yl)-[1,1′-biphenyl]-3-yl)-3-(((((R)-5-oxopyrrolidin-2-yl)methyl)amino)methyl)-4H-pyrido[1,2-a]pyrimidin-4-one;
8-(2′-chloro-2-methyl-3′-(5-(((((R)-5-oxopyrrolidin-2-yl)methyl)amino)methyl)-6-(trifluoromethyl)pyridin-2-yl)-[1,1′-biphenyl]-3-yl)-3-(((((R)-5-oxopyrrolidin-2-yl)methyl)amino)methyl)-4H-pyrido[1,2-a]pyrimidin-4-one;
8-(2′-chloro-2-methyl-3′-(6-methyl-5-(((((S)-5-oxopyrrolidin-2-yl)methyl)amino)methyl)pyridin-2-yl)-[1,1′-biphenyl]-3-yl)-3-(((((S)-5-oxopyrrolidin-2-yl)methyl)amino)methyl)-4H-pyrido[1,2-a]pyrimidin-4-one;
8-(2′-chloro-2-methyl-3′-(6-methyl-5-(((((R)-5-oxopyrrolidin-2-yl)methyl)amino)methyl)pyridin-2-yl)-[1,1′-biphenyl]-3-yl)-3-(((((S)-5-oxopyrrolidin-2-yl)methyl)amino)methyl)-4H-pyrido[1,2-a]pyrimidin-4-one;
8-(2′-chloro-2-methyl-3′-(6-methyl-5-(((((S)-5-oxopyrrolidin-2-yl)methyl)amino)methyl)pyridin-2-yl)-[1,1′-biphenyl]-3-yl)-3-(((((R)-5-oxopyrrolidin-2-yl)methyl)amino)methyl)-4H-pyrido[1,2-a]pyrimidin-4-one;
8-(2′-chloro-2-methyl-3′-(6-methyl-5-(((((R)-5-oxopyrrolidin-2-yl)methyl)amino)methyl)pyridin-2-yl)-[1,1′-biphenyl]-3-yl)-3-(((((R)-5-oxopyrrolidin-2-yl)methyl)amino)methyl)-4H-pyrido[1,2-a]pyrimidin-4-one;
(S)-8-(2,2′-dichloro-3′-(6-methoxy-5-((((5-oxopyrrolidin-2-yl)methyl)amino)methyl)pyridin-2-yl)-[1,1′-biphenyl]-3-yl)-3-(((2-hydroxyethyl)amino)methyl)-4H-pyrido[1,2-a]pyrimidin-4-one;
(R)-8-(2,2′-dichloro-3′-(6-methoxy-5-((((5-oxopyrrolidin-2-yl)methyl)amino)methyl)pyridin-2-yl)-[1,1′-biphenyl]-3-yl)-3-(((2-hydroxyethyl)amino)methyl)-4H-pyrido[1,2-a]pyrimidin-4-one;
(S)—N-(2-(((8-(2,2′-dichloro-3′-(6-methoxy-5-((((5-oxopyrrolidin-2-yl)methyl)amino)methyl)pyridin-2-yl)-[1,1′-biphenyl]-3-yl)-4-oxo-4H-pyrido[1,2-a]pyrimidin-3-yl)methyl)amino)ethyl)acetamide;
(R)—N-(2-(((8-(2,2′-dichloro-3′-(6-methoxy-5-((((5-oxopyrrolidin-2-yl)methyl)amino)methyl)pyridin-2-yl)-[1,1′-biphenyl]-3-yl)-4-oxo-4H-pyrido[1,2-a]pyrimidin-3-yl)methyl)amino)ethyl)acetamide;
(S)-5-((((6-(2,2′-dichloro-3′-(2-(((((S)-5-oxopyrrolidin-2-yl)methyl)amino)methyl)pyrazolo[1,5-a]pyrimidin-5-yl)-[1,1′-biphenyl]-3-yl)-2-methoxypyridin-3-yl)methyl)amino)methyl)pyrrolidin-2-one;
(R)-5-((((6-(2,2′-dichloro-3′-(2-(((((S)-5-oxopyrrolidin-2-yl)methyl)amino)methyl)pyrazolo[1,5-a]pyrimidin-5-yl)-[1,1′-biphenyl]-3-yl)-2-methoxypyridin-3-yl)methyl)amino)methyl)pyrrolidin-2-one;
(S)-5-((((6-(2,2′-dichloro-3′-(2-(((((R)-5-oxopyrrolidin-2-yl)methyl)amino)methyl)pyrazolo[1,5-a]pyrimidin-5-yl)-[1,1′-biphenyl]-3-yl)-2-methoxypyridin-3-yl)methyl)amino)methyl)pyrrolidin-2-one;
(R)-5-((((6-(2,2′-dichloro-3′-(2-(((((R)-5-oxopyrrolidin-2-yl)methyl)amino)methyl)pyrazolo[1,5-a]pyrimidin-5-yl)-[1,1′-biphenyl]-3-yl)-2-methoxypyridin-3-yl)methyl)amino)methyl)pyrrolidin-2-one;
(S)-1-((8-(2,2′-dichloro-3′-(5-((3-(hydroxymethyl)azetidin-1-yl)methyl)-6-methoxypyridin-2-yl)-[1,1′-biphenyl]-3-yl)-4-oxo-4H-pyrido[1,2-a]pyrimidin-3-yl)methyl)pyrrolidine-3-carboxylic acid;
(R)-1-((8-(2,2′-dichloro-3′-(5-((3-(hydroxymethyl)azetidin-1-yl)methyl)-6-methoxypyridin-2-yl)-[1,1′-biphenyl]-3-yl)-4-oxo-4H-pyrido[1,2-a]pyrimidin-3-yl)methyl)pyrrolidine-3-carboxylic acid;
1-((8-(2,2′-dichloro-3′-(5-((3-(hydroxymethyl)azetidin-1-yl)methyl)-6-methoxypyridin-2-yl)-[1,1′-biphenyl]-3-yl)-4-oxo-4H-pyrido[1,2-a]pyrimidin-3-yl)methyl)azetidine-3-carboxylic acid;
8-(2,2′-dichloro-3′-(6-methoxy-5-(((((S)-5-oxopyrrolidin-2-yl)methyl)amino)methyl)pyridin-2-yl)-[1,1′-biphenyl]-3-yl)-3-(((((S)-5-oxopyrrolidin-2-yl)methyl)amino)methyl)-4H-quinolizin-4-one;
8-(2,2′-dichloro-3′-(6-methoxy-5-(((((R)-5-oxopyrrolidin-2-yl)methyl)amino)methyl)pyridin-2-yl)-[1,1′-biphenyl]-3-yl)-3-(((((S)-5-oxopyrrolidin-2-yl)methyl)amino)methyl)-4H-quinolizin-4-one;
8-(2,2′-dichloro-3′-(6-methoxy-5-(((((S)-5-oxopyrrolidin-2-yl)methyl)amino)methyl)pyridin-2-yl)-[1,1′-biphenyl]-3-yl)-3-(((((R)-5-oxopyrrolidin-2-yl)methyl)amino)methyl)-4H-quinolizin-4-one;
8-(2,2′-dichloro-3′-(6-methoxy-5-(((((R)-5-oxopyrrolidin-2-yl)methyl)amino)methyl)pyridin-2-yl)-[1,1′-biphenyl]-3-yl)-3-(((((R)-5-oxopyrrolidin-2-yl)methyl)amino)methyl)-4H-quinolizin-4-one;
8-(2,2′-dichloro-5′-fluoro-3′-(6-methoxy-5-(((((S)-5-oxopyrrolidin-2-yl)methyl)amino)methyl)pyridin-2-yl)-[1,1′-biphenyl]-3-yl)-3-(((((S)-5-oxopyrrolidin-2-yl)methyl)amino)methyl)-4H-pyrido[1,2-a]pyrimidin-4-one;
8-(2,2′-dichloro-5′-fluoro-3′-(6-methoxy-5-(((((R)-5-oxopyrrolidin-2-yl)methyl)amino)methyl)pyridin-2-yl)-[1,1′-biphenyl]-3-yl)-3-(((((S)-5-oxopyrrolidin-2-yl)methyl)amino)methyl)-4H-pyrido[1,2-a]pyrimidin-4-one;
8-(2,2′-dichloro-5′-fluoro-3′-(6-methoxy-5-(((((S)-5-oxopyrrolidin-2-yl)methyl)amino)methyl)pyridin-2-yl)-[1,1′-biphenyl]-3-yl)-3-(((((R)-5-oxopyrrolidin-2-yl)methyl)amino)methyl)-4H-pyrido[1,2-a]pyrimidin-4-one;
8-(2,2′-dichloro-5′-fluoro-3′-(6-methoxy-5-(((((R)-5-oxopyrrolidin-2-yl)methyl)amino)methyl)pyridin-2-yl)-[1,1′-biphenyl]-3-yl)-3-(((((R)-5-oxopyrrolidin-2-yl)methyl)amino)methyl)-4H-pyrido[1,2-a]pyrimidin-4-one;
8-(2,2′-dichloro-4″-(((((S)-5-oxopyrrolidin-2-yl)methyl)amino)methyl)-3″-(trifluoromethoxy)-[1,1′:3′,1″-terphenyl]-3-yl)-3-(((((S)-5-oxopyrrolidin-2-yl)methyl)amino)methyl)-4H-pyrido[1,2-a]pyrimidin-4-one;
8-(2,2′-dichloro-4″-(((((R)-5-oxopyrrolidin-2-yl)methyl)amino)methyl)-3″-(trifluoromethoxy)-[1,1′:3′,1″-terphenyl]-3-yl)-3-(((((S)-5-oxopyrrolidin-2-yl)methyl)amino)methyl)-4H-pyrido[1,2-a]pyrimidin-4-one;
8-(2,2′-dichloro-4″-(((((S)-5-oxopyrrolidin-2-yl)methyl)amino)methyl)-3″-(trifluoromethoxy)-[1,1′:3′,1″-terphenyl]-3-yl)-3-(((((R)-5-oxopyrrolidin-2-yl)methyl)amino)methyl)-4H-pyrido[1,2-a]pyrimidin-4-one;
8-(2,2′-dichloro-4″-(((((R)-5-oxopyrrolidin-2-yl)methyl)amino)methyl)-3″-(trifluoromethoxy)-[1,1′:3′,1″-terphenyl]-3-yl)-3-(((((R)-5-oxopyrrolidin-2-yl)methyl)amino)methyl)-4H-pyrido[1,2-a]pyrimidin-4-one;
8-(2′-chloro-2-methyl-4″-(((((S)-5-oxopyrrolidin-2-yl)methyl)amino)methyl)-3″-(trifluoromethoxy)-[1,1′:3′,1″-terphenyl]-3-yl)-3-(((((S)-5-oxopyrrolidin-2-yl)methyl)amino)methyl)-4H-pyrido[1,2-a]pyrimidin-4-one;
8-(2′-chloro-2-methyl-4″-(((((R)-5-oxopyrrolidin-2-yl)methyl)amino)methyl)-3″-(trifluoromethoxy)-[1,1′:3′,1″-terphenyl]-3-yl)-3-(((((S)-5-oxopyrrolidin-2-yl)methyl)amino)methyl)-4H-pyrido[1,2-a]pyrimidin-4-one;
8-(2′-chloro-2-methyl-4″-(((((S)-5-oxopyrrolidin-2-yl)methyl)amino)methyl)-3″-(trifluoromethoxy)-[1,1′:3′,1″-terphenyl]-3-yl)-3-(((((R)-5-oxopyrrolidin-2-yl)methyl)amino)methyl)-4H-pyrido[1,2-a]pyrimidin-4-one;
8-(2′-chloro-2-methyl-4″-(((((R)-5-oxopyrrolidin-2-yl)methyl)amino)methyl)-3″-(trifluoromethoxy)-[1,1′:3′,1″-terphenyl]-3-yl)-3-(((((R)-5-oxopyrrolidin-2-yl)methyl)amino)methyl)-4H-pyrido[1,2-a]pyrimidin-4-one;
8-(2,2′-dichloro-3′-(6-(methylamino)-5-(((((S)-5-oxopyrrolidin-2-yl)methyl)amino)methyl)pyridin-2-yl)-[1,1′-biphenyl]-3-yl)-3-(((((S)-5-oxopyrrolidin-2-yl)methyl)amino)methyl)-4H-pyrido[1,2-a]pyrimidin-4-one;
8-(2,2′-dichloro-3′-(6-(methylamino)-5-(((((R)-5-oxopyrrolidin-2-yl)methyl)amino)methyl)pyridin-2-yl)-[1,1′-biphenyl]-3-yl)-3-(((((S)-5-oxopyrrolidin-2-yl)methyl)amino)methyl)-4H-pyrido[1,2-a]pyrimidin-4-one;
8-(2,2′-dichloro-3′-(6-(methylamino)-5-(((((S)-5-oxopyrrolidin-2-yl)methyl)amino)methyl)pyridin-2-yl)-[1,1′-biphenyl]-3-yl)-3-(((((R)-5-oxopyrrolidin-2-yl)methyl)amino)methyl)-4H-pyrido[1,2-a]pyrimidin-4-one;
8-(2,2′-dichloro-3′-(6-(methylamino)-5-(((((R)-5-oxopyrrolidin-2-yl)methyl)amino)methyl)pyridin-2-yl)-[1,1′-biphenyl]-3-yl)-3-(((((R)-5-oxopyrrolidin-2-yl)methyl)amino)methyl)-4H-pyrido[1,2-a]pyrimidin-4-one;
8-(2,2′-dichloro-3″-methoxy-4″-(((((S)-5-oxopyrrolidin-2-yl)methyl)amino)methyl)-[1,1′:3′,1″-terphenyl]-3-yl)-3-(((((S)-5-oxopyrrolidin-2-yl)methyl)amino)methyl)-4H-pyrido[1,2-a]pyrimidin-4-one;
8-(2,2′-dichloro-3″-methoxy-4″-(((((R)-5-oxopyrrolidin-2-yl)methyl)amino)methyl)-[1,1′:3′,1″-terphenyl]-3-yl)-3-(((((S)-5-oxopyrrolidin-2-yl)methyl)amino)methyl)-4H-pyrido[1,2-a]pyrimidin-4-one;
8-(2,2′-dichloro-3″-methoxy-4″-(((((S)-5-oxopyrrolidin-2-yl)methyl)amino)methyl)-[1,1′:3′,1″-terphenyl]-3-yl)-3-(((((R)-5-oxopyrrolidin-2-yl)methyl)amino)methyl)-4H-pyrido[1,2-a]pyrimidin-4-one;
8-(2,2′-dichloro-3″-methoxy-4″-(((((R)-5-oxopyrrolidin-2-yl)methyl)amino)methyl)-[1,1′:3′,1″-terphenyl]-3-yl)-3-(((((R)-5-oxopyrrolidin-2-yl)methyl)amino)methyl)-4H-pyrido[1,2-a]pyrimidin-4-one;
(S)-5-((((6-(2,2′-dichloro-3′-((S)-5-(((5-fluoropyridin-3-yl)methyl)amino)-8-methyl-5,6,7,8-tetrahydro-1,8-naphthyridin-2-yl)-[1,1′-biphenyl]-3-yl)-2-methoxypyridin-3-yl)methyl)amino)methyl)pyrrolidin-2-one;
(R)-5-((((6-(2,2′-dichloro-3′-((S)-5-(((5-fluoropyridin-3-yl)methyl)amino)-8-methyl-5,6,7,8-tetrahydro-1,8-naphthyridin-2-yl)-[1,1′-biphenyl]-3-yl)-2-methoxypyridin-3-yl)methyl)amino)methyl)pyrrolidin-2-one;
(S)-5-((((6-(2,2′-dichloro-3′-((R)-5-(((5-fluoropyridin-3-yl)methyl)amino)-8-methyl-5,6,7,8-tetrahydro-1,8-naphthyridin-2-yl)-[1,1′-biphenyl]-3-yl)-2-methoxypyridin-3-10 yl)methyl)amino)methyl)pyrrolidin-2-one;
(R)-5-((((6-(2,2′-dichloro-3′-((R)-5-(((5-fluoropyridin-3-yl)methyl)amino)-8-methyl-5,6,7,8-tetrahydro-1,8-naphthyridin-2-yl)-[1,1′-biphenyl]-3-yl)-2-methoxypyridin-3-yl)methyl)amino)methyl)pyrrolidin-2-one;
(S)-1-((8-(2,2′-dichloro-3′-(5-((3-(hydroxymethyl)azetidin-1-yl)methyl)-6-methoxypyrazin-2-yl)-[1,1′-biphenyl]-3-yl)-4-oxo-4H-pyrido[1,2-a]pyrimidin-3-yl)methyl)pyrrolidine-3-carboxylic acid;
(R)-1-((8-(2,2′-dichloro-3′-(5-((3-(hydroxymethyl)azetidin-1-yl)methyl)-6-methoxypyrazin-2-yl)-[1,1′-biphenyl]-3-yl)-4-oxo-4H-pyrido[1,2-a]pyrimidin-3-yl)methyl)pyrrolidine-3-carboxylic acid;
1-((8-(2,2′-dichloro-3′-(5-((3-(hydroxymethyl)azetidin-1-yl)methyl)-6-methoxypyrazin-2-yl)-[1,1′-biphenyl]-3-yl)-4-oxo-4H-pyrido[1,2-a]pyrimidin-3-yl)methyl)azetidine-3-carboxylic acid;
(S)-1-((8-(2,2′-dichloro-3′-(6-methoxy-5-(((((S)-5-oxopyrrolidin-2-yl)methyl)amino)methyl)pyridin-2-yl)-[1,1′-biphenyl]-3-yl)-4-oxo-4H-pyrido[1,2-a]pyrimidin-3-yl)methyl)pyrrolidine-3-carboxylic acid;
(R)-1-((8-(2,2′-dichloro-3′-(6-methoxy-5-(((((S)-5-oxopyrrolidin-2-yl)methyl)amino)methyl)pyridin-2-yl)-[1,1′-biphenyl]-3-yl)-4-oxo-4H-pyrido[1,2-a]pyrimidin-3-yl)methyl)pyrrolidine-3-carboxylic acid;
(S)-1-((8-(2,2′-dichloro-3′-(6-methoxy-5-(((((R)-5-oxopyrrolidin-2-yl)methyl)amino)methyl)pyridin-2-yl)-[1,1′-biphenyl]-3-yl)-4-oxo-4H-pyrido[1,2-a]pyrimidin-3-yl)methyl)pyrrolidine-3-carboxylic acid;
(R)-1-((8-(2,2′-dichloro-3′-(6-methoxy-5-(((((R)-5-oxopyrrolidin-2-yl)methyl)amino)methyl)pyridin-2-yl)-[1,1′-biphenyl]-3-yl)-4-oxo-4H-pyrido[1,2-a]pyrimidin-3-yl)methyl)pyrrolidine-3-carboxylic acid;
8-(2-chloro-3′-(6-methoxy-5-(((((S)-5-oxopyrrolidin-2-yl)methyl)amino)methyl)pyridin-2-yl)-2′-(trifluoromethyl)-[1,1′-biphenyl]-3-yl)-3-(((((S)-5-oxopyrrolidin-2-yl)methyl)amino)methyl)-4H-pyrido[1,2-a]pyrimidin-4-one;
8-(2-chloro-3′-(6-methoxy-5-(((((R)-5-oxopyrrolidin-2-yl)methyl)amino)methyl)pyridin-2-yl)-2′-(trifluoromethyl)-[1,1′-biphenyl]-3-yl)-3-(((((S)-5-oxopyrrolidin-2-yl)methyl)amino)methyl)-4H-pyrido[1,2-a]pyrimidin-4-one;
8-(2-chloro-3′-(6-methoxy-5-(((((S)-5-oxopyrrolidin-2-yl)methyl)amino)methyl)pyridin-2-yl)-2′-(trifluoromethyl)-[1,1′-biphenyl]-3-yl)-3-(((((R)-5-oxopyrrolidin-2-yl)methyl)amino)methyl)-4H-pyrido[1,2-a]pyrimidin-4-one;
8-(2-chloro-3′-(6-methoxy-5-(((((R)-5-oxopyrrolidin-2-yl)methyl)amino)methyl)pyridin-2-yl)-2′-(trifluoromethyl)-[1,1′-biphenyl]-3-yl)-3-(((((R)-5-oxopyrrolidin-2-yl)methyl)amino)methyl)-4H-pyrido[1,2-a]pyrimidin-4-one;
8-(3′-(6-methoxy-5-(((((S)-5-oxopyrrolidin-2-yl)methyl)amino)methyl)pyridin-2-yl)-2-methyl-2′-(trifluoromethyl)-[1,1′-biphenyl]-3-yl)-3-(((((S)-5-oxopyrrolidin-2-yl)methyl)amino)methyl)-4H-pyrido[1,2-a]pyrimidin-4-one;
8-(3′-(6-methoxy-5-(((((R)-5-oxopyrrolidin-2-yl)methyl)amino)methyl)pyridin-2-yl)-2-methyl-2′-(trifluoromethyl)-[1,1′-biphenyl]-3-yl)-3-(((((S)-5-oxopyrrolidin-2-yl)methyl)amino)methyl)-4H-pyrido[1,2-a]pyrimidin-4-one;
8-(3′-(6-methoxy-5-(((((S)-5-oxopyrrolidin-2-yl)methyl)amino)methyl)pyridin-2-yl)-2-methyl-2′-(trifluoromethyl)-[1,1′-biphenyl]-3-yl)-3-(((((R)-5-oxopyrrolidin-2-yl)methyl)amino)methyl)-4H-pyrido[1,2-a]pyrimidin-4-one;
8-(3′-(6-methoxy-5-(((((R)-5-oxopyrrolidin-2-yl)methyl)amino)methyl)pyridin-2-yl)-2-methyl-2′-(trifluoromethyl)-[1,1′-biphenyl]-3-yl)-3-(((((R)-5-oxopyrrolidin-2-yl)methyl)amino)methyl)-4H-pyrido[1,2-a]pyrimidin-4-one;
8-(2′-chloro-5′-fluoro-3′-(6-methoxy-5-(((((S)-5-oxopyrrolidin-2-yl)methyl)amino)methyl)pyridin-2-yl)-2-methyl-[1,1′-biphenyl]-3-yl)-3-(((((S)-5-oxopyrrolidin-2-yl)methyl)amino)methyl)-4H-pyrido[1,2-a]pyrimidin-4-one;
8-(2′-chloro-5′-fluoro-3′-(6-methoxy-5-(((((R)-5-oxopyrrolidin-2-yl)methyl)amino)methyl)pyridin-2-yl)-2-methyl-[1,1′-biphenyl]-3-yl)-3-(((((S)-5-oxopyrrolidin-2-yl)methyl)amino)methyl)-4H-pyrido[1,2-a]pyrimidin-4-one;
8-(2′-chloro-5′-fluoro-3′-(6-methoxy-5-(((((S)-5-oxopyrrolidin-2-yl)methyl)amino)methyl)pyridin-2-yl)-2-methyl-[1,1′-biphenyl]-3-yl)-3-(((((R)-5-oxopyrrolidin-2-yl)methyl)amino)methyl)-4H-pyrido[1,2-a]pyrimidin-4-one;
8-(2′-chloro-5′-fluoro-3′-(6-methoxy-5-(((((R)-5-oxopyrrolidin-2-yl)methyl)amino)methyl)pyridin-2-yl)-2-methyl-[1,1′-biphenyl]-3-yl)-3-(((((R)-5-oxopyrrolidin-2-yl)methyl)amino)methyl)-4H-pyrido[1,2-a]pyrimidin-4-one;
(S)-1-((8-(2,2′-dichloro-3′-(6-methoxy-5-((((5-oxopyrrolidin-2-yl)methyl)amino)methyl)pyridin-2-yl)-[1,1′-biphenyl]-3-yl)-4-oxo-4H-pyrido[1,2-a]pyrimidin-3-yl)methyl)azetidine-3-carboxylic acid;
(R)-1-((8-(2,2′-dichloro-3′-(6-methoxy-5-((((5-oxopyrrolidin-2-yl)methyl)amino)methyl)pyridin-2-yl)-[1,1′-biphenyl]-3-yl)-4-oxo-4H-pyrido[1,2-a]pyrimidin-3-yl)methyl)azetidine-3-carboxylic acid;
(S)-5-((((6-(2,2′-dichloro-3′-(6-(((((S)-5-oxopyrrolidin-2-yl)methyl)amino)methyl)imidazo[1,2-a]pyridin-2-yl)-[1,1′-biphenyl]-3-yl)-2-methoxypyridin-3-yl)methyl)amino)methyl)pyrrolidin-2-one;
(R)-5-((((6-(2,2′-dichloro-3′-(6-(((((S)-5-oxopyrrolidin-2-yl)methyl)amino)methyl)imidazo[1,2-a]pyridin-2-yl)-[1,1′-biphenyl]-3-yl)-2-methoxypyridin-3-yl)methyl)amino)methyl)pyrrolidin-2-one;
(S)-5-((((6-(2,2′-dichloro-3′-(6-(((((R)-5-oxopyrrolidin-2-yl)methyl)amino)methyl)imidazo[1,2-a]pyridin-2-yl)-[1,1′-biphenyl]-3-yl)-2-methoxypyridin-3-yl)methyl)amino)methyl)pyrrolidin-2-one;
(R)-5-((((6-(2,2′-dichloro-3′-(6-(((((R)-5-oxopyrrolidin-2-yl)methyl)amino)methyl)imidazo[1,2-a]pyridin-2-yl)-[1,1′-biphenyl]-3-yl)-2-methoxypyridin-3-yl)methyl)amino)methyl)pyrrolidin-2-one;
2-(((2-(2,2′-dichloro-3′-(5-(((2-hydroxyethyl)amino)methyl)-6-methoxypyridin-2-yl)-[1,1′-biphenyl]-3-yl)imidazo[1,2-a]pyridin-6-yl)methyl)amino)ethan-1-ol;
methyl ((8-(2,2′-dichloro-3′-(6-methoxy-5-(((((S)-5-oxopyrrolidin-2-yl)methyl)amino)methyl)pyridin-2-yl)-[1,1′-biphenyl]-3-yl)-4-oxo-4H-pyrido[1,2-a]pyrimidin-3-yl)methyl)-L-serinate;
methyl ((8-(2,2′-dichloro-3′-(6-methoxy-5-(((((R)-5-oxopyrrolidin-2-yl)methyl)amino)methyl)pyridin-2-yl)-[1,1′-biphenyl]-3-yl)-4-oxo-4H-pyrido[1,2-a]pyrimidin-3-yl)methyl)-L-serinate;
methyl ((8-(2,2′-dichloro-3′-(6-methoxy-5-(((((S)-5-oxopyrrolidin-2-yl)methyl)amino)methyl)pyridin-2-yl)-[1,1′-biphenyl]-3-yl)-4-oxo-4H-pyrido[1,2-a]pyrimidin-3-yl)methyl)-D-serinate;
methyl ((8-(2,2′-dichloro-3′-(6-methoxy-5-(((((R)-5-oxopyrrolidin-2-yl)methyl)amino)methyl)pyridin-2-yl)-[1,1′-biphenyl]-3-yl)-4-oxo-4H-pyrido[1,2-a]pyrimidin-3-yl)methyl)-D-serinate;
(S)-8-(2,2′-dichloro-3′-(5-(((2-hydroxyethyl)amino)methyl)-6-methoxypyridin-2-yl)-[1,1′-biphenyl]-3-yl)-3-((((5-oxopyrrolidin-2-yl)methyl)amino)methyl)-4H-pyrido[1,2-a]pyrimidin-4-one;
(R)-8-(2,2′-dichloro-3′-(5-(((2-hydroxyethyl)amino)methyl)-6-methoxypyridin-2-yl)-[1,1′-biphenyl]-3-yl)-3-((((5-oxopyrrolidin-2-yl)methyl)amino)methyl)-4H-pyrido[1,2-a]pyrimidin-4-one;
(S)—N-(2-(((6-(2,2′-dichloro-3′-(4-oxo-3-((((5-oxopyrrolidin-2-yl)methyl)amino)methyl)-4H-pyrido[1,2-a]pyrimidin-8-yl)-[1,1′-biphenyl]-3-yl)-2-methoxypyridin-3-yl)methyl)amino)ethyl)acetamide;
(R)—N-(2-(((6-(2,2′-dichloro-3′-(4-oxo-3-((((5-oxopyrrolidin-2-yl)methyl)amino)methyl)-4H-pyrido[1,2-a]pyrimidin-8-yl)-[1,1′-biphenyl]-3-yl)-2-methoxypyridin-3-yl)methyl)amino)ethyl)acetamide;
8-(2,2′-dichloro-3″-fluoro-5″-methoxy-4″-(((((S)-5-oxopyrrolidin-2-yl)methyl)amino)methyl)-[1,1′:3′,1″-terphenyl]-3-yl)-3-(((((S)-5-oxopyrrolidin-2-yl)methyl)amino)methyl)-4H-pyrido[1,2-a]pyrimidin-4-one;
8-(2,2′-dichloro-3″-fluoro-5″-methoxy-4″-(((((R)-5-oxopyrrolidin-2-yl)methyl)amino)methyl)-[1,1′:3′,1″-terphenyl]-3-yl)-3-(((((S)-5-oxopyrrolidin-2-yl)methyl)amino)methyl)-4H-pyrido[1,2-a]pyrimidin-4-one;
8-(2,2′-dichloro-3″-fluoro-5″-methoxy-4″-(((((S)-5-oxopyrrolidin-2-yl)methyl)amino)methyl)-[1,1′:3′,1″-terphenyl]-3-yl)-3-(((((R)-5-oxopyrrolidin-2-yl)methyl)amino)methyl)-4H-pyrido[1,2-a]pyrimidin-4-one;
8-(2,2′-dichloro-3″-fluoro-5″-methoxy-4″-(((((R)-5-oxopyrrolidin-2-yl)methyl)amino)methyl)-[1,1′:3′,1″-terphenyl]-3-yl)-3-(((((R)-5-oxopyrrolidin-2-yl)methyl)amino)methyl)-4H-pyrido[1,2-a]pyrimidin-4-one;
8-(2,2′-dichloro-2″-fluoro-3″-methoxy-4″-(((((S)-5-oxopyrrolidin-2-yl)methyl)amino)methyl)-[1,1′:3′,1″-terphenyl]-3-yl)-3-(((((S)-5-oxopyrrolidin-2-yl)methyl)amino)methyl)-4H-pyrido[1,2-a]pyrimidin-4-one;
8-(2,2′-dichloro-2″-fluoro-3″-methoxy-4″-(((((R)-5-oxopyrrolidin-2-yl)methyl)amino)methyl)-[1,1′:3′,1″-terphenyl]-3-yl)-3-(((((S)-5-oxopyrrolidin-2-yl)methyl)amino)methyl)-4H-pyrido[1,2-a]pyrimidin-4-one;
8-(2,2′-dichloro-2″-fluoro-3″-methoxy-4″-(((((S)-5-oxopyrrolidin-2-yl)methyl)amino)methyl)-[1,1′:3′,1″-terphenyl]-3-yl)-3-(((((R)-5-oxopyrrolidin-2-yl)methyl)amino)methyl)-4H-pyrido[1,2-a]pyrimidin-4-one;
8-(2,2′-dichloro-2″-fluoro-3″-methoxy-4″-(((((R)-5-oxopyrrolidin-2-yl)methyl)amino)methyl)-[1,1′:3′,1″-terphenyl]-3-yl)-3-(((((R)-5-oxopyrrolidin-2-yl)methyl)amino)methyl)-4H-pyrido[1,2-a]pyrimidin-4-one;
8-(2,2′-dichloro-2″-fluoro-5″-methoxy-4″-(((((S)-5-oxopyrrolidin-2-yl)methyl)amino)methyl)-[1,1′:3′,1″-terphenyl]-3-yl)-3-(((((S)-5-oxopyrrolidin-2-yl)methyl)amino)methyl)-4H-pyrido[1,2-a]pyrimidin-4-one;
8-(2,2′-dichloro-2″-fluoro-5″-methoxy-4″-(((((R)-5-oxopyrrolidin-2-yl)methyl)amino)methyl)-[1,1′:3′,1″-terphenyl]-3-yl)-3-(((((S)-5-oxopyrrolidin-2-yl)methyl)amino)methyl)-4H-pyrido[1,2-a]pyrimidin-4-one;
8-(2,2′-dichloro-2″-fluoro-5″-methoxy-4″-(((((S)-5-oxopyrrolidin-2-yl)methyl)amino)methyl)-[1,1′:3′,1″-terphenyl]-3-yl)-3-(((((R)-5-oxopyrrolidin-2-yl)methyl)amino)methyl)-4H-pyrido[1,2-a]pyrimidin-4-one;
8-(2,2′-dichloro-2″-fluoro-5″-methoxy-4″-(((((R)-5-oxopyrrolidin-2-yl)methyl)amino)methyl)-[1,1′:3′,1″-terphenyl]-3-yl)-3-(((((R)-5-oxopyrrolidin-2-yl)methyl)amino)methyl)-4H-pyrido[1,2-a]pyrimidin-4-one;
8-(2,2′-dichloro-3′-(6-methoxy-5-(((((S)-5-oxopyrrolidin-2-yl)methyl)amino)methyl)pyrazin-2-yl)-[1,1′-biphenyl]-3-yl)-3-(((((S)-5-oxopyrrolidin-2-yl)methyl)amino)methyl)-4H-quinolizin-4-one;
8-(2,2′-dichloro-3′-(6-methoxy-5-(((((R)-5-oxopyrrolidin-2-yl)methyl)amino)methyl)pyrazin-2-yl)-[1,1′-biphenyl]-3-yl)-3-(((((S)-5-oxopyrrolidin-2-yl)methyl)amino)methyl)-4H-quinolizin-4-one;
8-(2,2′-dichloro-3′-(6-methoxy-5-(((((S)-5-oxopyrrolidin-2-yl)methyl)amino)methyl)pyrazin-2-yl)-[1,1′-biphenyl]-3-yl)-3-(((((R)-5-oxopyrrolidin-2-yl)methyl)amino)methyl)-4H-quinolizin-4-one;
8-(2,2′-dichloro-3′-(6-methoxy-5-(((((R)-5-oxopyrrolidin-2-yl)methyl)amino)methyl)pyrazin-2-yl)-[1,1′-biphenyl]-3-yl)-3-(((((R)-5-oxopyrrolidin-2-yl)methyl)amino)methyl)-4H-quinolizin-4-one;
8-(2,2′-dichloro-3′-(6-(methylthio)-5-(((((S)-5-oxopyrrolidin-2-yl)methyl)amino)methyl)pyridin-2-yl)-[1,1′-biphenyl]-3-yl)-3-(((((S)-5-oxopyrrolidin-2-yl)methyl)amino)methyl)-4H-pyrido[1,2-a]pyrimidin-4-one;
8-(2,2′-dichloro-3′-(6-(methylthio)-5-(((((R)-5-oxopyrrolidin-2-yl)methyl)amino)methyl)pyridin-2-yl)-[1,1′-biphenyl]-3-yl)-3-(((((S)-5-oxopyrrolidin-2-yl)methyl)amino)methyl)-4H-pyrido[1,2-a]pyrimidin-4-one;
8-(2,2′-dichloro-3′-(6-(methylthio)-5-(((((S)-5-oxopyrrolidin-2-yl)methyl)amino)methyl)pyridin-2-yl)-[1,1′-biphenyl]-3-yl)-3-(((((R)-5-oxopyrrolidin-2-yl)methyl)amino)methyl)-4H-pyrido[1,2-a]pyrimidin-4-one;
8-(2,2′-dichloro-3′-(6-(methylthio)-5-(((((R)-5-oxopyrrolidin-2-yl)methyl)amino)methyl)pyridin-2-yl)-[1,1′-biphenyl]-3-yl)-3-(((((R)-5-oxopyrrolidin-2-yl)methyl)amino)methyl)-4H-pyrido[1,2-a]pyrimidin-4-one;
8-(2,2′-dichloro-3′-(6-cyclopropoxy-5-(((((S)-5-oxopyrrolidin-2-yl)methyl)amino)methyl)pyridin-2-yl)-[1,1′-biphenyl]-3-yl)-3-(((((S)-5-oxopyrrolidin-2-yl)methyl)amino)methyl)-4H-pyrido[1,2-a]pyrimidin-4-one;
8-(2,2′-dichloro-3′-(6-cyclopropoxy-5-(((((R)-5-oxopyrrolidin-2-yl)methyl)amino)methyl)pyridin-2-yl)-[1,1′-biphenyl]-3-yl)-3-(((((S)-5-oxopyrrolidin-2-yl)methyl)amino)methyl)-4H-pyrido[1,2-a]pyrimidin-4-one;
8-(2,2′-dichloro-3′-(6-cyclopropoxy-5-(((((S)-5-oxopyrrolidin-2-yl)methyl)amino)methyl)pyridin-2-yl)-[1,1′-biphenyl]-3-yl)-3-(((((R)-5-oxopyrrolidin-2-yl)methyl)amino)methyl)-4H-pyrido[1,2-a]pyrimidin-4-one;
8-(2,2′-dichloro-3′-(6-cyclopropoxy-5-(((((R)-5-oxopyrrolidin-2-yl)methyl)amino)methyl)pyridin-2-yl)-[1,1′-biphenyl]-3-yl)-3-(((((R)-5-oxopyrrolidin-2-yl)methyl)amino)methyl)-4H-pyrido[1,2-a]pyrimidin-4-one;
8-(2,2′-dichloro-3′-(difluoromethoxy)-4″-(((((S)-5-oxopyrrolidin-2-yl)methyl)amino)methyl)-[1,1′:3′,1″-terphenyl]-3-yl)-3-(((((S)-5-oxopyrrolidin-2-yl)methyl)amino)methyl)-4H-pyrido[1,2-a]pyrimidin-4-one;
8-(2,2′-dichloro-3′-(difluoromethoxy)-4″-(((((R)-5-oxopyrrolidin-2-yl)methyl)amino)methyl)-[1,1′:3′,1″-terphenyl]-3-yl)-3-(((((S)-5-oxopyrrolidin-2-yl)methyl)amino)methyl)-4H-pyrido[1,2-a]pyrimidin-4-one;
8-(2,2′-dichloro-3′-(difluoromethoxy)-4″-(((((S)-5-oxopyrrolidin-2-yl)methyl)amino)methyl)-[1,1′:3′,1″-terphenyl]-3-yl)-3-(((((R)-5-oxopyrrolidin-2-yl)methyl)amino)methyl)-4H-pyrido[1,2-a]pyrimidin-4-one;
8-(2,2′-dichloro-3′-(difluoromethoxy)-4″-(((((R)-5-oxopyrrolidin-2-yl)methyl)amino)methyl)-[1,1′:3′,1″-terphenyl]-3-yl)-3-(((((R)-5-oxopyrrolidin-2-yl)methyl)amino)methyl)-4H-pyrido[1,2-a]pyrimidin-4-one;
(S)-8-(2,2′-dichloro-3′-(6-methoxy-5-((((5-oxopyrrolidin-2-yl)methyl)amino)methyl)pyridin-2-yl)-[1,1′-biphenyl]-3-yl)-3-((3-(2-hydroxypropan-2-yl)azetidin-1-yl)methyl)-4H-pyrido[1,2-a]pyrimidin-4-one;
(R)-8-(2,2′-dichloro-3′-(6-methoxy-5-((((5-oxopyrrolidin-2-yl)methyl)amino)methyl)pyridin-2-yl)-[1,1′-biphenyl]-3-yl)-3-((3-(2-hydroxypropan-2-yl)azetidin-1-yl)methyl)-4H-pyrido[1,2-a]pyrimidin-4-one;
(S)-8-(2,2′-dichloro-3′-(5-((3-(2-hydroxypropan-2-yl)azetidin-1-yl)methyl)-6-methoxypyridin-2-yl)-[1,1′-biphenyl]-3-yl)-3-((((5-oxopyrrolidin-2-yl)methyl)amino)methyl)-4H-pyrido[1,2-a]pyrimidin-4-one;
(R)-8-(2,2′-dichloro-3′-(5-((3-(2-hydroxypropan-2-yl)azetidin-1-yl)methyl)-6-methoxypyridin-2-yl)-[1,1′-biphenyl]-3-yl)-3-((((5-oxopyrrolidin-2-yl)methyl)amino)methyl)-4H-pyrido[1,2-a]pyrimidin-4-one;
methyl ((6-(2,2′-dichloro-3′-(4-oxo-3-(((((S)-5-oxopyrrolidin-2-yl)methyl)amino)methyl)-4H-pyrido[1,2-a]pyrimidin-8-yl)-[1,1′-biphenyl]-3-yl)-2-methoxypyridin-3-yl)methyl)-L-serinate;
methyl ((6-(2,2′-dichloro-3′-(4-oxo-3-(((((R)-5-oxopyrrolidin-2-yl)methyl)amino)methyl)-4H-pyrido[1,2-a]pyrimidin-8-yl)-[1,1′-biphenyl]-3-yl)-2-methoxypyridin-3-yl)methyl)-L-serinate;
methyl ((6-(2,2′-dichloro-3′-(4-oxo-3-(((((S)-5-oxopyrrolidin-2-yl)methyl)amino)methyl)-4H-pyrido[1,2-a]pyrimidin-8-yl)-[1,1′-biphenyl]-3-yl)-2-methoxypyridin-3-yl)methyl)-D-serinate;
methyl ((6-(2,2′-dichloro-3′-(4-oxo-3-(((((R)-5-oxopyrrolidin-2-yl)methyl)amino)methyl)-4H-pyrido[1,2-a]pyrimidin-8-yl)-[1,1′-biphenyl]-3-yl)-2-methoxypyridin-3-yl)methyl)-D-serinate;
((6-(2,2′-dichloro-3′-(4-oxo-3-(((((S)-5-oxopyrrolidin-2-yl)methyl)amino)methyl)-4H-pyrido[1,2-a]pyrimidin-8-yl)-[1,1′-biphenyl]-3-yl)-2-methoxypyridin-3-yl)methyl)-L-serine;
((6-(2,2′-dichloro-3′-(4-oxo-3-(((((R)-5-oxopyrrolidin-2-yl)methyl)amino)methyl)-4H-pyrido[1,2-a]pyrimidin-8-yl)-[1,1′-biphenyl]-3-yl)-2-methoxypyridin-3-yl)methyl)-L-serine;
((6-(2,2′-dichloro-3′-(4-oxo-3-(((((S)-5-oxopyrrolidin-2-yl)methyl)amino)methyl)-4H-pyrido[1,2-a]pyrimidin-8-yl)-[1,1′-biphenyl]-3-yl)-2-methoxypyridin-3-yl)methyl)-D-serine;
((6-(2,2′-dichloro-3′-(4-oxo-3-(((((R)-5-oxopyrrolidin-2-yl)methyl)amino)methyl)-4H-pyrido[1,2-a]pyrimidin-8-yl)-[1,1′-biphenyl]-3-yl)-2-methoxypyridin-3-yl)methyl)-D-serine;
(S)-1-(((6-(2,2′-dichloro-3′-(4-oxo-3-((((5-oxopyrrolidin-2-yl)methyl)amino)methyl)-4H-pyrido[1,2-a]pyrimidin-8-yl)-[1,1′-biphenyl]-3-yl)-2-methoxypyridin-3-yl)methyl)amino)cyclopropane-1-carboxylic acid;
(R)-1-(((6-(2,2′-dichloro-3′-(4-oxo-3-((((5-oxopyrrolidin-2-yl)methyl)amino)methyl)-4H-pyrido[1,2-a]pyrimidin-8-yl)-[1,1′-biphenyl]-3-yl)-2-methoxypyridin-3-yl)methyl)amino)cyclopropane-1-carboxylic acid;
(S)-1-((8-(2,2′-dichloro-3′-(6-methoxy-5-(((((S)-5-oxopyrrolidin-2-yl)methyl)amino)methyl)pyrazin-2-yl)-[1,1′-biphenyl]-3-yl)-4-oxo-4H-pyrido[1,2-a]pyrimidin-3-yl)methyl)pyrrolidine-3-carboxylic acid;
(R)-1-((8-(2,2′-dichloro-3′-(6-methoxy-5-(((((S)-5-oxopyrrolidin-2-yl)methyl)amino)methyl)pyrazin-2-yl)-[1,1′-biphenyl]-3-yl)-4-oxo-4H-pyrido[1,2-a]pyrimidin-3-yl)methyl)pyrrolidine-3-carboxylic acid;
(S)-1-((8-(2,2′-dichloro-3′-(6-methoxy-5-(((((R)-5-oxopyrrolidin-2-yl)methyl)amino)methyl)pyrazin-2-yl)-[1,1′-biphenyl]-3-yl)-4-oxo-4H-pyrido[1,2-a]pyrimidin-3-yl)methyl)pyrrolidine-3-carboxylic acid;
(R)-1-((8-(2,2′-dichloro-3′-(6-methoxy-5-(((((R)-5-oxopyrrolidin-2-yl)methyl)amino)methyl)pyrazin-2-yl)-[1,1′-biphenyl]-3-yl)-4-oxo-4H-pyrido[1,2-a]pyrimidin-3-yl)methyl)pyrrolidine-3-carboxylic acid;
(S)-1-((8-(2,2′-dichloro-3′-(6-methoxy-5-((((5-oxopyrrolidin-2-yl)methyl)amino)methyl)pyrazin-2-yl)-[1,1′-biphenyl]-3-yl)-4-oxo-4H-pyrido[1,2-a]pyrimidin-3-yl)methyl)azetidine-3-carboxylic acid;
(R)-1-((8-(2,2′-dichloro-3′-(6-methoxy-5-((((5-oxopyrrolidin-2-yl)methyl)amino)methyl)pyrazin-2-yl)-[1,1′-biphenyl]-3-yl)-4-oxo-4H-pyrido[1,2-a]pyrimidin-3-yl)methyl)azetidine-3-carboxylic acid;
(S)-1-((6-(2,2′-dichloro-3′-(4-oxo-3-(((((S)-5-oxopyrrolidin-2-yl)methyl)amino)methyl)-4H-pyrido[1,2-a]pyrimidin-8-yl)-[1,1′-biphenyl]-3-yl)-2-methoxypyridin-3-yl)methyl)pyrrolidine-3-carboxylic acid;
(R)-1-((6-(2,2′-dichloro-3′-(4-oxo-3-(((((S)-5-oxopyrrolidin-2-yl)methyl)amino)methyl)-4H-pyrido[1,2-a]pyrimidin-8-yl)-[1,1′-biphenyl]-3-yl)-2-methoxypyridin-3-yl)methyl)pyrrolidine-3-carboxylic acid;
(S)-1-((6-(2,2′-dichloro-3′-(4-oxo-3-(((((R)-5-oxopyrrolidin-2-yl)methyl)amino)methyl)-4H-pyrido[1,2-a]pyrimidin-8-yl)-[1,1′-biphenyl]-3-yl)-2-methoxypyridin-3-yl)methyl)pyrrolidine-3-carboxylic acid;
(R)-1-((6-(2,2′-dichloro-3′-(4-oxo-3-(((((R)-5-oxopyrrolidin-2-yl)methyl)amino)methyl)-4H-pyrido[1,2-a]pyrimidin-8-yl)-[1,1′-biphenyl]-3-yl)-2-methoxypyridin-3-yl)methyl)pyrrolidine-3-carboxylic acid;
(S)-1-((6-(2,2′-dichloro-3′-(4-oxo-3-((((5-oxopyrrolidin-2-yl)methyl)amino)methyl)-4H-pyrido[1,2-a]pyrimidin-8-yl)-[1,1′-biphenyl]-3-yl)-2-methoxypyridin-3-yl)methyl)azetidine-3-carboxylic acid;
(R)-1-((6-(2,2′-dichloro-3′-(4-oxo-3-((((5-oxopyrrolidin-2-yl)methyl)amino)methyl)-4H-pyrido[1,2-a]pyrimidin-8-yl)-[1,1′-biphenyl]-3-yl)-2-methoxypyridin-3-yl)methyl)azetidine-3-carboxylic acid;
(S)-1-(((8-(2,2′-dichloro-3′-(6-methoxy-5-((((5-oxopyrrolidin-2-yl)methyl)amino)methyl)pyridin-2-yl)-[1,1′-biphenyl]-3-yl)-4-oxo-4H-pyrido[1,2-a]pyrimidin-3-yl)methyl)amino)cyclopropane-1-carboxylic acid;
(R)-1-(((8-(2,2′-dichloro-3′-(6-methoxy-5-((((5-oxopyrrolidin-2-yl)methyl)amino)methyl)pyridin-2-yl)-[1,1′-biphenyl]-3-yl)-4-oxo-4H-pyrido[1,2-a]pyrimidin-3-yl)methyl)amino)cyclopropane-1-carboxylic acid;
methyl (S)-2-((6-(2,2′-dichloro-3′-(4-oxo-3-((((5-oxopyrrolidin-2-yl)methyl)amino)methyl)-4H-pyrido[1,2-a]pyrimidin-8-yl)-[1,1′-biphenyl]-3-yl)-2-methoxypyridin-3-yl)methyl)-2-azabicyclo[2.2.2]octane-4-carboxylate;
methyl (R)-2-((6-(2,2′-dichloro-3′-(4-oxo-3-((((5-oxopyrrolidin-2-yl)methyl)amino)methyl)-4H-pyrido[1,2-a]pyrimidin-8-yl)-[1,1′-biphenyl]-3-yl)-2-methoxypyridin-3-yl)methyl)-2-azabicyclo[2.2.2]octane-4-carboxylate;
(S)-2-((6-(2,2′-dichloro-3′-(4-oxo-3-((((5-oxopyrrolidin-2-yl)methyl)amino)methyl)-4H-pyrido[1,2-a]pyrimidin-8-yl)-[1,1′-biphenyl]-3-yl)-2-methoxypyridin-3-yl)methyl)-2-azabicyclo[2.2.2]octane-4-carboxylic acid;
(R)-2-((6-(2,2′-dichloro-3′-(4-oxo-3-((((5-oxopyrrolidin-2-yl)methyl)amino)methyl)-4H-pyrido[1,2-a]pyrimidin-8-yl)-[1,1′-biphenyl]-3-yl)-2-methoxypyridin-3-yl)methyl)-2-azabicyclo[2.2.2]octane-4-carboxylic acid;
(S)-5-((((3-chloro-6-(2,2′-dichloro-3′-(6-methoxy-5-(((((S)-5-oxopyrrolidin-2-yl)methyl)amino)methyl)pyridin-2-yl)-[1,1′-biphenyl]-3-yl)thieno[3,2-b]pyridin-2-yl)methyl)amino)methyl)pyrrolidin-2-one;
(R)-5-((((3-chloro-6-(2,2′-dichloro-3′-(6-methoxy-5-(((((S)-5-oxopyrrolidin-2-yl)methyl)amino)methyl)pyridin-2-yl)-[1,1′-biphenyl]-3-yl)thieno[3,2-b]pyridin-2-yl)methyl)amino)methyl)pyrrolidin-2-one;
(S)-5-((((3-chloro-6-(2,2′-dichloro-3′-(6-methoxy-5-(((((R)-5-oxopyrrolidin-2-yl)methyl)amino)methyl)pyridin-2-yl)-[1,1′-biphenyl]-3-yl)thieno[3,2-b]pyridin-2-yl)methyl)amino)methyl)pyrrolidin-2-one;
(R)-5-((((3-chloro-6-(2,2′-dichloro-3′-(6-methoxy-5-(((((R)-5-oxopyrrolidin-2-yl)methyl)amino)methyl)pyridin-2-yl)-[1,1′-biphenyl]-3-yl)thieno[3,2-b]pyridin-2-yl)methyl)amino)methyl)pyrrolidin-2-one;
methyl (S)-((6-(2,2′-dichloro-3′-(4-oxo-3-((((5-oxopyrrolidin-2-yl)methyl)amino)methyl)-4H-pyrido[1,2-a]pyrimidin-8-yl)-[1,1′-biphenyl]-3-yl)-2-methoxypyridin-3-yl)methyl)glycinate;
methyl (R)-((6-(2,2′-dichloro-3′-(4-oxo-3-((((5-oxopyrrolidin-2-yl)methyl)amino)methyl)-4H-pyrido[1,2-a]pyrimidin-8-yl)-[1,1′-biphenyl]-3-yl)-2-methoxypyridin-3-yl)methyl)glycinate;
(S)-((6-(2,2′-dichloro-3′-(4-oxo-3-((((5-oxopyrrolidin-2-yl)methyl)amino)methyl)-4H-pyrido[1,2-a]pyrimidin-8-yl)-[1,1′-biphenyl]-3-yl)-2-methoxypyridin-3-yl)methyl)glycine;
(R)-((6-(2,2′-dichloro-3′-(4-oxo-3-((((5-oxopyrrolidin-2-yl)methyl)amino)methyl)-4H-pyrido[1,2-a]pyrimidin-8-yl)-[1,1′-biphenyl]-3-yl)-2-methoxypyridin-3-yl)methyl)glycine;
(S)-4-(((8-(2,2′-dichloro-3′-(6-methoxy-5-(((((S)-5-oxopyrrolidin-2-yl)methyl)amino)methyl)pyridin-2-yl)-[1,1′-biphenyl]-3-yl)-4-oxo-4H-pyrido[1,2-a]pyrimidin-3-yl)methyl)amino)-3-hydroxybutanoic acid;
(R)-4-(((8-(2,2′-dichloro-3′-(6-methoxy-5-(((((S)-5-oxopyrrolidin-2-yl)methyl)amino)methyl)pyridin-2-yl)-[1,1′-biphenyl]-3-yl)-4-oxo-4H-pyrido[1,2-a]pyrimidin-3-yl)methyl)amino)-3-hydroxybutanoic acid;
(S)-4-(((8-(2,2′-dichloro-3′-(6-methoxy-5-(((((R)-5-oxopyrrolidin-2-yl)methyl)amino)methyl)pyridin-2-yl)-[1,1′-biphenyl]-3-yl)-4-oxo-4H-pyrido[1,2-a]pyrimidin-3-yl)methyl)amino)-3-hydroxybutanoic acid;
(R)-4-(((8-(2,2′-dichloro-3′-(6-methoxy-5-(((((R)-5-oxopyrrolidin-2-yl)methyl)amino)methyl)pyridin-2-yl)-[1,1′-biphenyl]-3-yl)-4-oxo-4H-pyrido[1,2-a]pyrimidin-3-yl)methyl)amino)-3-hydroxybutanoic acid;
(S)-2-((8-(2,2′-dichloro-3′-(6-methoxy-5-((((5-oxopyrrolidin-2-yl)methyl)amino)methyl)pyridin-2-yl)-[1,1′-biphenyl]-3-yl)-4-oxo-4H-pyrido[1,2-a]pyrimidin-3-yl)methyl)-2-azabicyclo[2.2.2]octane-4-carboxylic acid;
(R)-2-((8-(2,2′-dichloro-3′-(6-methoxy-5-((((5-oxopyrrolidin-2-yl)methyl)amino)methyl)pyridin-2-yl)-[1,1′-biphenyl]-3-yl)-4-oxo-4H-pyrido[1,2-a]pyrimidin-3-yl)methyl)-2-azabicyclo[2.2.2]octane-4-carboxylic acid;
((8-(2,2′-dichloro-3′-(6-methoxy-5-(((((S)-5-oxopyrrolidin-2-yl)methyl)amino)methyl)pyridin-2-yl)-[1,1′-biphenyl]-3-yl)-4-oxo-4H-pyrido[1,2-a]pyrimidin-3-yl)methyl)-L-proline;
((8-(2,2′-dichloro-3′-(6-methoxy-5-(((((R)-5-oxopyrrolidin-2-yl)methyl)amino)methyl)pyridin-2-yl)-[1,1′-biphenyl]-3-yl)-4-oxo-4H-pyrido[1,2-a]pyrimidin-3-yl)methyl)-D-proline;
((8-(2,2′-dichloro-3′-(6-methoxy-5-(((((S)-5-oxopyrrolidin-2-yl)methyl)amino)methyl)pyridin-2-yl)-[1,1′-biphenyl]-3-yl)-4-oxo-4H-pyrido[1,2-a]pyrimidin-3-yl)methyl)-D-proline;
((8-(2,2′-dichloro-3′-(6-methoxy-5-(((((R)-5-oxopyrrolidin-2-yl)methyl)amino)methyl)pyridin-2-yl)-[1,1′-biphenyl]-3-yl)-4-oxo-4H-pyrido[1,2-a]pyrimidin-3-yl)methyl)-L-proline;
(S)-1-((8-(2,2′-dichloro-3′-(6-methoxy-5-(((((S)-5-oxopyrrolidin-2-yl)methyl)amino)methyl)pyridin-2-yl)-[1,1′-biphenyl]-3-yl)-4-oxo-4H-pyrido[1,2-a]pyrimidin-3-yl)methyl)piperidine-2-carboxylic acid;
(R)-1-((8-(2,2′-dichloro-3′-(6-methoxy-5-(((((S)-5-oxopyrrolidin-2-yl)methyl)amino)methyl)pyridin-2-yl)-[1,1′-biphenyl]-3-yl)-4-oxo-4H-pyrido[1,2-a]pyrimidin-3-yl)methyl)piperidine-2-carboxylic acid;
(S)-1-((8-(2,2′-dichloro-3′-(6-methoxy-5-(((((R)-5-oxopyrrolidin-2-yl)methyl)amino)methyl)pyridin-2-yl)-[1,1′-biphenyl]-3-yl)-4-oxo-4H-pyrido[1,2-a]pyrimidin-3-yl)methyl)piperidine-2-carboxylic acid;
(R)-1-((8-(2,2′-dichloro-3′-(6-methoxy-5-(((((R)-5-oxopyrrolidin-2-yl)methyl)amino)methyl)pyridin-2-yl)-[1,1′-biphenyl]-3-yl)-4-oxo-4H-pyrido[1,2-a]pyrimidin-3-yl)methyl)piperidine-2-carboxylic acid;
(S)-1-((8-(2,2′-dichloro-3′-(6-methoxy-5-(((((S)-5-oxopyrrolidin-2-yl)methyl)amino)methyl)pyridin-2-yl)-[1,1′-biphenyl]-3-yl)-4-oxo-4H-pyrido[1,2-a]pyrimidin-3-yl)methyl)piperidine-3-carboxylic acid;
(R)-1-((8-(2,2′-dichloro-3′-(6-methoxy-5-(((((S)-5-oxopyrrolidin-2-yl)methyl)amino)methyl)pyridin-2-yl)-[1,1′-biphenyl]-3-yl)-4-oxo-4H-pyrido[1,2-a]pyrimidin-3-yl)methyl)piperidine-3-carboxylic acid;
(S)-1-((8-(2,2′-dichloro-3′-(6-methoxy-5-(((((R)-5-oxopyrrolidin-2-yl)methyl)amino)methyl)pyridin-2-yl)-[1,1′-biphenyl]-3-yl)-4-oxo-4H-pyrido[1,2-a]pyrimidin-3-yl)methyl)piperidine-3-carboxylic acid;
(R)-1-((8-(2,2′-dichloro-3′-(6-methoxy-5-(((((R)-5-oxopyrrolidin-2-yl)methyl)amino)methyl)pyridin-2-yl)-[1,1′-biphenyl]-3-yl)-4-oxo-4H-pyrido[1,2-a]pyrimidin-3-yl)methyl)piperidine-3-carboxylic acid;
8-(2,2′-dichloro-3′-(6-methoxy-5-(((((S)-5-oxopyrrolidin-2-yl)methyl)amino)methyl)pyridin-2-yl)-[1,1′-biphenyl]-3-yl)-3-((((S)-2-hydroxypropyl)amino)methyl)-4H-pyrido[1,2-a]pyrimidin-4-one;
8-(2,2′-dichloro-3′-(6-methoxy-5-(((((R)-5-oxopyrrolidin-2-yl)methyl)amino)methyl)pyridin-2-yl)-[1,1′-biphenyl]-3-yl)-3-((((S)-2-hydroxypropyl)amino)methyl)-4H-pyrido[1,2-a]pyrimidin-4-one;
8-(2,2′-dichloro-3′-(6-methoxy-5-(((((S)-5-oxopyrrolidin-2-yl)methyl)amino)methyl)pyridin-2-yl)-[1,1′-biphenyl]-3-yl)-3-((((R)-2-hydroxypropyl)amino)methyl)-4H-pyrido[1,2-a]pyrimidin-4-one;
8-(2,2′-dichloro-3′-(6-methoxy-5-(((((R)-5-oxopyrrolidin-2-yl)methyl)amino)methyl)pyridin-2-yl)-[1,1′-biphenyl]-3-yl)-3-((((R)-2-hydroxypropyl)amino)methyl)-4H-pyrido[1,2-a]pyrimidin-4-one;
8-(2,2′-dichloro-3′-(6-methoxy-5-(((((S)-5-oxopyrrolidin-2-yl)methyl)amino)methyl)pyridin-2-yl)-[1,1′-biphenyl]-3-yl)-3-((((R)-2-hydroxypropyl)amino)methyl)-4H-pyrido[1,2-a]pyrimidin-4-one;
8-(2,2′-dichloro-3′-(6-methoxy-5-(((((R)-5-oxopyrrolidin-2-yl)methyl)amino)methyl)pyridin-2-yl)-[1,1′-biphenyl]-3-yl)-3-((((R)-2-hydroxypropyl)amino)methyl)-4H-pyrido[1,2-a]pyrimidin-4-one;
8-(2,2′-dichloro-3′-(6-methoxy-5-(((((S)-5-oxopyrrolidin-2-yl)methyl)amino)methyl)pyridin-2-yl)-[1,1′-biphenyl]-3-yl)-3-((((S)-2-hydroxypropyl)amino)methyl)-4H-pyrido[1,2-a]pyrimidin-4-one;
8-(2,2′-dichloro-3′-(6-methoxy-5-(((((R)-5-oxopyrrolidin-2-yl)methyl)amino)methyl)pyridin-2-yl)-[1,1′-biphenyl]-3-yl)-3-((((S)-2-hydroxypropyl)amino)methyl)-4H-pyrido[1,2-a]pyrimidin-4-one;
(S)-8-(2,2′-dichloro-3′-(6-methoxy-5-((((5-oxopyrrolidin-2-yl)methyl)amino)methyl)pyridin-2-yl)-[1,1′-biphenyl]-3-yl)-3-(((2-hydroxy-2-methylpropyl)amino)methyl)-4H-pyrido[1,2-a]pyrimidin-4-one;
(R)-8-(2,2′-dichloro-3′-(6-methoxy-5-((((5-oxopyrrolidin-2-yl)methyl)amino)methyl)pyridin-2-yl)-[1,1′-biphenyl]-3-yl)-3-(((2-hydroxy-2-methylpropyl)amino)methyl)-4H-pyrido[1,2-a]pyrimidin-4-one;
(2R,4R)-1-((8-(2,2′-dichloro-3′-(6-methoxy-5-(((((S)-5-oxopyrrolidin-2-yl)methyl)amino)methyl)pyridin-2-yl)-[1,1′-biphenyl]-3-yl)-4-oxo-4H-pyrido[1,2-a]pyrimidin-3-yl)methyl)-4-hydroxypyrrolidine-2-carboxylic acid;
(2R,4S)-1-((8-(2,2′-dichloro-3′-(6-methoxy-5-(((((S)-5-oxopyrrolidin-2-yl)methyl)amino)methyl)pyridin-2-yl)-[1,1′-biphenyl]-3-yl)-4-oxo-4H-pyrido[1,2-a]pyrimidin-3-yl)methyl)-4-hydroxypyrrolidine-2-carboxylic acid;
(2S,4R)-1-((8-(2,2′-dichloro-3′-(6-methoxy-5-(((((S)-5-oxopyrrolidin-2-yl)methyl)amino)methyl)pyridin-2-yl)-[1,1′-biphenyl]-3-yl)-4-oxo-4H-pyrido[1,2-a]pyrimidin-3-yl)methyl)-4-hydroxypyrrolidine-2-carboxylic acid;
(2S,4S)-1-((8-(2,2′-dichloro-3′-(6-methoxy-5-(((((S)-5-oxopyrrolidin-2-yl)methyl)amino)methyl)pyridin-2-yl)-[1,1′-biphenyl]-3-yl)-4-oxo-4H-pyrido[1,2-a]pyrimidin-3-yl)methyl)-4-hydroxypyrrolidine-2-carboxylic acid;
(2R,4R)-1-((8-(2,2′-dichloro-3′-(6-methoxy-5-(((((R)-5-oxopyrrolidin-2-yl)methyl)amino)methyl)pyridin-2-yl)-[1,1′-biphenyl]-3-yl)-4-oxo-4H-pyrido[1,2-a]pyrimidin-3-yl)methyl)-4-hydroxypyrrolidine-2-carboxylic acid;
(2R,4S)-1-((8-(2,2′-dichloro-3′-(6-methoxy-5-(((((R)-5-oxopyrrolidin-2-yl)methyl)amino)methyl)pyridin-2-yl)-[1,1′-biphenyl]-3-yl)-4-oxo-4H-pyrido[1,2-a]pyrimidin-3-yl)methyl)-4-hydroxypyrrolidine-2-carboxylic acid;
(2S,4R)-1-((8-(2,2′-dichloro-3′-(6-methoxy-5-(((((R)-5-oxopyrrolidin-2-yl)methyl)amino)methyl)pyridin-2-yl)-[1,1′-biphenyl]-3-yl)-4-oxo-4H-pyrido[1,2-a]pyrimidin-3-yl)methyl)-4-hydroxypyrrolidine-2-carboxylic acid;
(2S,4S)-1-((8-(2,2′-dichloro-3′-(6-methoxy-5-(((((R)-5-oxopyrrolidin-2-yl)methyl)amino)methyl)pyridin-2-yl)-[1,1′-biphenyl]-3-yl)-4-oxo-4H-pyrido[1,2-a]pyrimidin-3-yl)methyl)-4-hydroxypyrrolidine-2-carboxylic acid;
(S)-1-((6-(2,2′-dichloro-3′-(3-((3-(hydroxymethyl)azetidin-1-yl)methyl)-4-oxo-4H-pyrido[1,2-a]pyrimidin-8-yl)-[1,1′-biphenyl]-3-yl)-2-methoxypyridin-3-yl)methyl)pyrrolidine-3-carboxylic acid;
(R)-1-((6-(2,2′-dichloro-3′-(3-((3-(hydroxymethyl)azetidin-1-yl)methyl)-4-oxo-4H-pyrido[1,2-a]pyrimidin-8-yl)-[1,1′-biphenyl]-3-yl)-2-methoxypyridin-3-yl)methyl)pyrrolidine-3-carboxylic acid;
1-((6-(2,2′-dichloro-3′-(3-((3-(hydroxymethyl)azetidin-1-yl)methyl)-4-oxo-4H-pyrido[1,2-a]pyrimidin-8-yl)-[1,1′-biphenyl]-3-yl)-2-methoxypyridin-3-yl)methyl)azetidine-3-carboxylic acid;
methyl (S)-4-(((8-(2,2′-dichloro-3′-(6-methoxy-5-((((5-oxopyrrolidin-2-yl)methyl)amino)methyl)pyridin-2-yl)-[1,1′-biphenyl]-3-yl)-4-oxo-4H-pyrido[1,2-a]pyrimidin-3-yl)methyl)amino)butanoate;
methyl (R)-4-(((8-(2,2′-dichloro-3′-(6-methoxy-5-((((5-oxopyrrolidin-2-yl)methyl)amino)methyl)pyridin-2-yl)-[1,1′-biphenyl]-3-yl)-4-oxo-4H-pyrido[1,2-a]pyrimidin-3-yl)methyl)amino)butanoate;
(S)-8-(2,2′-dichloro-3′-(6-methoxy-5-((((5-oxopyrrolidin-2-yl)methyl)amino)methyl)pyridin-2-yl)-[1,1′-biphenyl]-3-yl)-3-(((2,4-dimethoxybenzyl)amino)methyl)-4H-pyrido[1,2-a]pyrimidin-4-one;
(R)-8-(2,2′-dichloro-3′-(6-methoxy-5-((((5-oxopyrrolidin-2-yl)methyl)amino)methyl)pyridin-2-yl)-[1,1′-biphenyl]-3-yl)-3-(((2,4-dimethoxybenzyl)amino)methyl)-4H-pyrido[1,2-a]pyrimidin-4-one;
(S)-2-(1-(((8-(2,2′-dichloro-3′-(6-methoxy-5-((((5-oxopyrrolidin-2-yl)methyl)amino)methyl)pyridin-2-yl)-[1,1′-biphenyl]-3-yl)-4-oxo-4H-pyrido[1,2-a]pyrimidin-3-yl)methyl)amino)cyclopropyl)acetic acid;
(R)-2-(1-(((8-(2,2′-dichloro-3′-(6-methoxy-5-((((5-oxopyrrolidin-2-yl)methyl)amino)methyl)pyridin-2-yl)-[1,1′-biphenyl]-3-yl)-4-oxo-4H-pyrido[1,2-a]pyrimidin-3-yl)methyl)amino)cyclopropyl)acetic acid;
8-(2,2′-dichloro-5-fluoro-3′-(6-methoxy-5-(((((S)-5-oxopyrrolidin-2-yl)methyl)amino)methyl)pyridin-2-yl)-[1,1′-biphenyl]-3-yl)-3-(((((S)-5-oxopyrrolidin-2-yl)methyl)amino)methyl)-4H-pyrido[1,2-a]pyrimidin-4-one;
8-(2,2′-dichloro-5-fluoro-3′-(6-methoxy-5-(((((R)-5-oxopyrrolidin-2-yl)methyl)amino)methyl)pyridin-2-yl)-[1,1′-biphenyl]-3-yl)-3-(((((S)-5-oxopyrrolidin-2-yl)methyl)amino)methyl)-4H-pyrido[1,2-a]pyrimidin-4-one;
8-(2,2′-dichloro-5-fluoro-3′-(6-methoxy-5-(((((S)-5-oxopyrrolidin-2-yl)methyl)amino)methyl)pyridin-2-yl)-[1,1′-biphenyl]-3-yl)-3-(((((R)-5-oxopyrrolidin-2-yl)methyl)amino)methyl)-4H-pyrido[1,2-a]pyrimidin-4-one;
8-(2,2′-dichloro-5-fluoro-3′-(6-methoxy-5-(((((R)-5-oxopyrrolidin-2-yl)methyl)amino)methyl)pyridin-2-yl)-[1,1′-biphenyl]-3-yl)-3-(((((R)-5-oxopyrrolidin-2-yl)methyl)amino)methyl)-4H-pyrido[1,2-a]pyrimidin-4-one;
3-(((((S)-5-oxopyrrolidin-2-yl)methyl)amino)methyl)-8-(2,2′,5-trichloro-3′-(6-methoxy-5-(((((S)-5-oxopyrrolidin-2-yl)methyl)amino)methyl)pyridin-2-yl)-[1,1′-biphenyl]-3-yl)-4H-pyrido[1,2-a]pyrimidin-4-one;
3-(((((R)-5-oxopyrrolidin-2-yl)methyl)amino)methyl)-8-(2,2′,5-trichloro-3′-(6-methoxy-5-(((((S)-5-oxopyrrolidin-2-yl)methyl)amino)methyl)pyridin-2-yl)-[1,1′-biphenyl]-3-yl)-4H-pyrido[1,2-a]pyrimidin-4-one;
3-(((((S)-5-oxopyrrolidin-2-yl)methyl)amino)methyl)-8-(2,2′,5-trichloro-3′-(6-methoxy-5-(((((R)-5-oxopyrrolidin-2-yl)methyl)amino)methyl)pyridin-2-yl)-[1,1′-biphenyl]-3-yl)-4H-pyrido[1,2-a]pyrimidin-4-one;
3-(((((R)-5-oxopyrrolidin-2-yl)methyl)amino)methyl)-8-(2,2′,5-trichloro-3′-(6-methoxy-5-(((((R)-5-oxopyrrolidin-2-yl)methyl)amino)methyl)pyridin-2-yl)-[1,1′-biphenyl]-3-yl)-4H-pyrido[1,2-a]pyrimidin-4-one;
8-(2,2′-dichloro-3′-(5-((((1r,3r)-3-hydroxy-3-methylcyclobutyl)amino)methyl)-6-methoxypyridin-2-yl)-[1,1′-biphenyl]-3-yl)-3-(((((S)-5-oxopyrrolidin-2-yl)methyl)amino)methyl)-4H-pyrido[1,2-a]pyrimidin-4-one;
8-(2,2′-dichloro-3′-(5-((((1r,3r)-3-hydroxy-3-methylcyclobutyl)amino)methyl)-6-methoxypyridin-2-yl)-[1,1′-biphenyl]-3-yl)-3-(((((R)-5-oxopyrrolidin-2-yl)methyl)amino)methyl)-4H-pyrido[1,2-a]pyrimidin-4-one;
8-(2,2′-dichloro-3′-(5-((((1s,3s)-3-hydroxy-3-methylcyclobutyl)amino)methyl)-6-methoxypyridin-2-yl)-[1,1′-biphenyl]-3-yl)-3-(((((S)-5-oxopyrrolidin-2-yl)methyl)amino)methyl)-4H-pyrido[1,2-a]pyrimidin-4-one;
8-(2,2′-dichloro-3′-(5-((((1s,3s)-3-hydroxy-3-methylcyclobutyl)amino)methyl)-6-methoxypyridin-2-yl)-[1,1′-biphenyl]-3-yl)-3-(((((R)-5-oxopyrrolidin-2-yl)methyl)amino)methyl)-4H-pyrido[1,2-a]pyrimidin-4-one;
8-(2,2′-dichloro-3′-(6-methoxy-5-(((((S)-5-oxopyrrolidin-2-yl)methyl)amino)methyl)pyridin-2-yl)-[1,1′-biphenyl]-3-yl)-3-((((1r,3r)-3-hydroxy-3-methylcyclobutyl)amino)methyl)-4H-pyrido[1,2-a]pyrimidin-4-one;
8-(2,2′-dichloro-3′-(6-methoxy-5-(((((R)-5-oxopyrrolidin-2-yl)methyl)amino)methyl)pyridin-2-yl)-[1,1′-biphenyl]-3-yl)-3-((((1s,3s)-3-hydroxy-3-methylcyclobutyl)amino)methyl)-4H-pyrido[1,2-a]pyrimidin-4-one;
8-(2,2′-dichloro-3′-(6-methoxy-5-(((((S)-5-oxopyrrolidin-2-yl)methyl)amino)methyl)pyridin-2-yl)-[1,1′-biphenyl]-3-yl)-3-((((1s,3s)-3-hydroxy-3-methylcyclobutyl)amino)methyl)-4H-pyrido[1,2-a]pyrimidin-4-one;
8-(2,2′-dichloro-3′-(6-methoxy-5-(((((R)-5-oxopyrrolidin-2-yl)methyl)amino)methyl)pyridin-2-yl)-[1,1′-biphenyl]-3-yl)-3-((((1r,3r)-3-hydroxy-3-methylcyclobutyl)amino)methyl)-4H-pyrido[1,2-a]pyrimidin-4-one;
8-(2,2′-dichloro-3′-(6-methoxy-5-(((((S)-5-oxopyrrolidin-2-yl)methyl)amino)methyl)pyridin-2-yl)-[1,1′-biphenyl]-3-yl)-3-(((S)-3-hydroxypyrrolidin-1-yl)methyl)-4H-pyrido[1,2-a]pyrimidin-4-one;
8-(2,2′-dichloro-3′-(6-methoxy-5-(((((R)-5-oxopyrrolidin-2-yl)methyl)amino)methyl)pyridin-2-yl)-[1,1′-biphenyl]-3-yl)-3-(((S)-3-hydroxypyrrolidin-1-yl)methyl)-4H-pyrido[1,2-a]pyrimidin-4-one;
8-(2,2′-dichloro-3′-(6-methoxy-5-(((((S)-5-oxopyrrolidin-2-yl)methyl)amino)methyl)pyridin-2-yl)-[1,1′-biphenyl]-3-yl)-3-(((R)-3-hydroxypyrrolidin-1-yl)methyl)-4H-pyrido[1,2-a]pyrimidin-4-one;
8-(2,2′-dichloro-3′-(6-methoxy-5-(((((R)-5-oxopyrrolidin-2-yl)methyl)amino)methyl)pyridin-2-yl)-[1,1′-biphenyl]-3-yl)-3-(((R)-3-hydroxypyrrolidin-1-yl)methyl)-4H-pyrido[1,2-a]pyrimidin-4-one;
2′-chloro-3-(6-methoxy-5-(((((S)-5-oxopyrrolidin-2-yl)methyl)amino)methyl)pyridin-2-yl)-3′-(4-oxo-3-(((((S)-5-oxopyrrolidin-2-yl)methyl)amino)methyl)-4H-pyrido[1,2-a]pyrimidin-8-yl)-[1,1′-biphenyl]-2-carbonitrile;
2′-chloro-3-(6-methoxy-5-(((((R)-5-oxopyrrolidin-2-yl)methyl)amino)methyl)pyridin-2-yl)-3′-(4-oxo-3-(((((S)-5-oxopyrrolidin-2-yl)methyl)amino)methyl)-4H-pyrido[1,2-a]pyrimidin-8-yl)-[1,1′-biphenyl]-2-carbonitrile;
2′-chloro-3-(6-methoxy-5-(((((S)-5-oxopyrrolidin-2-yl)methyl)amino)methyl)pyridin-2-yl)-3′-(4-oxo-3-(((((R)-5-oxopyrrolidin-2-yl)methyl)amino)methyl)-4H-pyrido[1,2-a]pyrimidin-8-yl)-[1,1′-biphenyl]-2-carbonitrile;
2′-chloro-3-(6-methoxy-5-(((((R)-5-oxopyrrolidin-2-yl)methyl)amino)methyl)pyridin-2-yl)-3′-(4-oxo-3-(((((R)-5-oxopyrrolidin-2-yl)methyl)amino)methyl)-4H-pyrido[1,2-a]pyrimidin-8-yl)-[1,1′-biphenyl]-2-carbonitrile;
8-(2,2′-dichloro-5′-methoxy-3′-(6-methoxy-5-(((((S)-5-oxopyrrolidin-2-yl)methyl)amino)methyl)pyridin-2-yl)-[1,1′-biphenyl]-3-yl)-3-(((((S)-5-oxopyrrolidin-2-yl)methyl)amino)methyl)-4H-pyrido[1,2-a]pyrimidin-4-one;
8-(2,2′-dichloro-5′-methoxy-3′-(6-methoxy-5-(((((R)-5-oxopyrrolidin-2-yl)methyl)amino)methyl)pyridin-2-yl)-[1,1′-biphenyl]-3-yl)-3-(((((S)-5-oxopyrrolidin-2-yl)methyl)amino)methyl)-4H-pyrido[1,2-a]pyrimidin-4-one;
8-(2,2′-dichloro-5′-methoxy-3′-(6-methoxy-5-(((((S)-5-oxopyrrolidin-2-yl)methyl)amino)methyl)pyridin-2-yl)-[1,1′-biphenyl]-3-yl)-3-(((((R)-5-oxopyrrolidin-2-yl)methyl)amino)methyl)-4H-pyrido[1,2-a]pyrimidin-4-one;
8-(2,2′-dichloro-5′-methoxy-3′-(6-methoxy-5-(((((R)-5-oxopyrrolidin-2-yl)methyl)amino)methyl)pyridin-2-yl)-[1,1′-biphenyl]-3-yl)-3-(((((R)-5-oxopyrrolidin-2-yl)methyl)amino)methyl)-4H-pyrido[1,2-a]pyrimidin-4-one;
8-(2,2′-dichloro-3′-(4-fluoro-6-methoxy-5-(((((S)-5-oxopyrrolidin-2-yl)methyl)amino)methyl)pyridin-2-yl)-[1,1′-biphenyl]-3-yl)-3-(((((S)-5-oxopyrrolidin-2-yl)methyl)amino)methyl)-4H-pyrido[1,2-a]pyrimidin-4-one;
8-(2,2′-dichloro-3′-(4-fluoro-6-methoxy-5-(((((R)-5-oxopyrrolidin-2-yl)methyl)amino)methyl)pyridin-2-yl)-[1,1′-biphenyl]-3-yl)-3-(((((S)-5-oxopyrrolidin-2-yl)methyl)amino)methyl)-4H-pyrido[1,2-a]pyrimidin-4-one;
8-(2,2′-dichloro-3′-(4-fluoro-6-methoxy-5-(((((S)-5-oxopyrrolidin-2-yl)methyl)amino)methyl)pyridin-2-yl)-[1,1′-biphenyl]-3-yl)-3-(((((R)-5-oxopyrrolidin-2-yl)methyl)amino)methyl)-4H-pyrido[1,2-a]pyrimidin-4-one;
8-(2,2′-dichloro-3′-(4-fluoro-6-methoxy-5-(((((R)-5-oxopyrrolidin-2-yl)methyl)amino)methyl)pyridin-2-yl)-[1,1′-biphenyl]-3-yl)-3-(((((R)-5-oxopyrrolidin-2-yl)methyl)amino)methyl)-4H-pyrido[1,2-a]pyrimidin-4-one;
(2S,4S)-1-((8-(2,2′-dichloro-3′-(6-methoxy-5-(((((S)-5-oxopyrrolidin-2-yl)methyl)amino)methyl)pyridin-2-yl)-[1,1′-biphenyl]-3-yl)-4-oxo-4H-pyrido[1,2-a]pyrimidin-3-yl)methyl)-4-hydroxypyrrolidine-2-carboxylic acid;
(2R,4S)-1-((8-(2,2′-dichloro-3′-(6-methoxy-5-(((((S)-5-oxopyrrolidin-2-yl)methyl)amino)methyl)pyridin-2-yl)-[1,1′-biphenyl]-3-yl)-4-oxo-4H-pyrido[1,2-a]pyrimidin-3-yl)methyl)-4-hydroxypyrrolidine-2-carboxylic acid;
(2S,4R)-1-((8-(2,2′-dichloro-3′-(6-methoxy-5-(((((S)-5-oxopyrrolidin-2-yl)methyl)amino)methyl)pyridin-2-yl)-[1,1′-biphenyl]-3-yl)-4-oxo-4H-pyrido[1,2-a]pyrimidin-3-yl)methyl)-4-hydroxypyrrolidine-2-carboxylic acid;
(2R,4R)-1-((8-(2,2′-dichloro-3′-(6-methoxy-5-(((((S)-5-oxopyrrolidin-2-yl)methyl)amino)methyl)pyridin-2-yl)-[1,1′-biphenyl]-3-yl)-4-oxo-4H-pyrido[1,2-a]pyrimidin-3-yl)methyl)-4-hydroxypyrrolidine-2-carboxylic acid;
(2S,4S)-1-((8-(2,2′-dichloro-3′-(6-methoxy-5-(((((R)-5-oxopyrrolidin-2-yl)methyl)amino)methyl)pyridin-2-yl)-[1,1′-biphenyl]-3-yl)-4-oxo-4H-pyrido[1,2-a]pyrimidin-3-yl)methyl)-4-hydroxypyrrolidine-2-carboxylic acid;
(2R,4S)-1-((8-(2,2′-dichloro-3′-(6-methoxy-5-(((((R)-5-oxopyrrolidin-2-yl)methyl)amino)methyl)pyridin-2-yl)-[1,1′-biphenyl]-3-yl)-4-oxo-4H-pyrido[1,2-a]pyrimidin-3-yl)methyl)-4-hydroxypyrrolidine-2-carboxylic acid;
(2S,4R)-1-((8-(2,2′-dichloro-3′-(6-methoxy-5-(((((R)-5-oxopyrrolidin-2-yl)methyl)amino)methyl)pyridin-2-yl)-[1,1′-biphenyl]-3-yl)-4-oxo-4H-pyrido[1,2-a]pyrimidin-3-yl)methyl)-4-hydroxypyrrolidine-2-carboxylic acid;
(2R,4R)-1-((8-(2,2′-dichloro-3′-(6-methoxy-5-(((((R)-5-oxopyrrolidin-2-yl)methyl)amino)methyl)pyridin-2-yl)-[1,1′-biphenyl]-3-yl)-4-oxo-4H-pyrido[1,2-a]pyrimidin-3-yl)methyl)-4-hydroxypyrrolidine-2-carboxylic acid;
(2R,4S)-1-((8-(2,2′-dichloro-3′-(6-methoxy-5-(((((S)-5-oxopyrrolidin-2-yl)methyl)amino)methyl)pyridin-2-yl)-[1,1′-biphenyl]-3-yl)-4-oxo-4H-pyrido[1,2-a]pyrimidin-3-yl)methyl)-4-hydroxypyrrolidine-2-carboxylic acid;
(2S,4S)-1-((8-(2,2′-dichloro-3′-(6-methoxy-5-(((((S)-5-oxopyrrolidin-2-yl)methyl)amino)methyl)pyridin-2-yl)-[1,1′-biphenyl]-3-yl)-4-oxo-4H-pyrido[1,2-a]pyrimidin-3-yl)methyl)-4-hydroxypyrrolidine-2-carboxylic acid;
(2R,4R)-1-((8-(2,2′-dichloro-3′-(6-methoxy-5-(((((S)-5-oxopyrrolidin-2-yl)methyl)amino)methyl)pyridin-2-yl)-[1,1′-biphenyl]-3-yl)-4-oxo-4H-pyrido[1,2-a]pyrimidin-3-yl)methyl)-4-hydroxypyrrolidine-2-carboxylic acid;
(2S,4R)-1-((8-(2,2′-dichloro-3′-(6-methoxy-5-(((((S)-5-oxopyrrolidin-2-yl)methyl)amino)methyl)pyridin-2-yl)-[1,1′-biphenyl]-3-yl)-4-oxo-4H-pyrido[1,2-a]pyrimidin-3-yl)methyl)-4-hydroxypyrrolidine-2-carboxylic acid;
(2R,4S)-1-((8-(2,2′-dichloro-3′-(6-methoxy-5-(((((R)-5-oxopyrrolidin-2-yl)methyl)amino)methyl)pyridin-2-yl)-[1,1′-biphenyl]-3-yl)-4-oxo-4H-pyrido[1,2-a]pyrimidin-3-yl)methyl)-4-hydroxypyrrolidine-2-carboxylic acid;
(2S,4S)-1-((8-(2,2′-dichloro-3′-(6-methoxy-5-(((((R)-5-oxopyrrolidin-2-yl)methyl)amino)methyl)pyridin-2-yl)-[1,1′-biphenyl]-3-yl)-4-oxo-4H-pyrido[1,2-a]pyrimidin-3-yl)methyl)-4-hydroxypyrrolidine-2-carboxylic acid;
(2R,4R)-1-((8-(2,2′-dichloro-3′-(6-methoxy-5-(((((R)-5-oxopyrrolidin-2-yl)methyl)amino)methyl)pyridin-2-yl)-[1,1′-biphenyl]-3-yl)-4-oxo-4H-pyrido[1,2-a]pyrimidin-3-yl)methyl)-4-hydroxypyrrolidine-2-carboxylic acid;
(2S,4R)-1-((8-(2,2′-dichloro-3′-(6-methoxy-5-(((((R)-5-oxopyrrolidin-2-yl)methyl)amino)methyl)pyridin-2-yl)-[1,1′-biphenyl]-3-yl)-4-oxo-4H-pyrido[1,2-a]pyrimidin-3-yl)methyl)-4-hydroxypyrrolidine-2-carboxylic acid;
N-((8-(2,2′-dichloro-3′-(6-methoxy-5-(((((S)-5-oxopyrrolidin-2-yl)methyl)amino)methyl)pyridin-2-yl)-[1,1′-biphenyl]-3-yl)-4-oxo-4H-pyrido[1,2-a]pyrimidin-3-yl)methyl)-O-methyl-D-serine;
N-((8-(2,2′-dichloro-3′-(6-methoxy-5-(((((R)-5-oxopyrrolidin-2-yl)methyl)amino)methyl)pyridin-2-yl)-[1,1′-biphenyl]-3-yl)-4-oxo-4H-pyrido[1,2-a]pyrimidin-3-yl)methyl)-O-methyl-D-serine;
N-((8-(2,2′-dichloro-3′-(6-methoxy-5-(((((S)-5-oxopyrrolidin-2-yl)methyl)amino)methyl)pyridin-2-yl)-[1,1′-biphenyl]-3-yl)-4-oxo-4H-pyrido[1,2-a]pyrimidin-3-yl)methyl)-O-methyl-L-serine;
N-((8-(2,2′-dichloro-3′-(6-methoxy-5-(((((R)-5-oxopyrrolidin-2-yl)methyl)amino)methyl)pyridin-2-yl)-[1,1′-biphenyl]-3-yl)-4-oxo-4H-pyrido[1,2-a]pyrimidin-3-yl)methyl)-O-methyl-L-serine;
((8-(2,2′-dichloro-3′-(6-methoxy-5-(((((S)-5-oxopyrrolidin-2-yl)methyl)amino)methyl)pyridin-2-yl)-[1,1′-biphenyl]-3-yl)-4-oxo-4H-pyrido[1,2-a]pyrimidin-3-yl)methyl)-L-serine;
((8-(2,2′-dichloro-3′-(6-methoxy-5-(((((R)-5-oxopyrrolidin-2-yl)methyl)amino)methyl)pyridin-2-yl)-[1,1′-biphenyl]-3-yl)-4-oxo-4H-pyrido[1,2-a]pyrimidin-3-yl)methyl)-L-serine;
((8-(2,2′-dichloro-3′-(6-methoxy-5-(((((S)-5-oxopyrrolidin-2-yl)methyl)amino)methyl)pyridin-2-yl)-[1,1′-biphenyl]-3-yl)-4-oxo-4H-pyrido[1,2-a]pyrimidin-3-yl)methyl)-D-serine;
((8-(2,2′-dichloro-3′-(6-methoxy-5-(((((R)-5-oxopyrrolidin-2-yl)methyl)amino)methyl)pyridin-2-yl)-[1,1′-biphenyl]-3-yl)-4-oxo-4H-pyrido[1,2-a]pyrimidin-3-yl)methyl)-D-serine;
8-(2,2′-dichloro-3′-(6-methoxy-5-(((((S)-5-oxopyrrolidin-2-yl)methyl)amino)methyl)pyridin-2-yl)-[1,1′-biphenyl]-3-yl)-3-(((S)-6-oxohexahydropyrrolo[1,2-a]pyrazin-2(1H)-yl)methyl)-4H-pyrido[1,2-a]pyrimidin-4-one;
8-(2,2′-dichloro-3′-(6-methoxy-5-(((((R)-5-oxopyrrolidin-2-yl)methyl)amino)methyl)pyridin-2-yl)-[1,1′-biphenyl]-3-yl)-3-(((S)-6-oxohexahydropyrrolo[1,2-a]pyrazin-2(1H)-yl)methyl)-4H-pyrido[1,2-a]pyrimidin-4-one;
8-(2,2′-dichloro-3′-(6-methoxy-5-(((((S)-5-oxopyrrolidin-2-yl)methyl)amino)methyl)pyridin-2-yl)-[1,1′-biphenyl]-3-yl)-3-(((R)-6-oxohexahydropyrrolo[1,2-a]pyrazin-2(1H)-yl)methyl)-4H-pyrido[1,2-a]pyrimidin-4-one;
8-(2,2′-dichloro-3′-(6-methoxy-5-(((((R)-5-oxopyrrolidin-2-yl)methyl)amino)methyl)pyridin-2-yl)-[1,1′-biphenyl]-3-yl)-3-(((R)-6-oxohexahydropyrrolo[1,2-a]pyrazin-2(1H)-yl)methyl)-4H-pyrido[1,2-a]pyrimidin-4-one;
8-(2,2′-dichloro-3′-(6-methoxy-5-(((((S)-5-oxopyrrolidin-2-yl)methyl)amino)methyl)pyridin-2-yl)-[1,1′-biphenyl]-3-yl)-3-(((S)-2-(hydroxymethyl)pyrrolidin-1-yl)methyl)-4H-pyrido[1,2-a]pyrimidin-4-one;
8-(2,2′-dichloro-3′-(6-methoxy-5-(((((R)-5-oxopyrrolidin-2-yl)methyl)amino)methyl)pyridin-2-yl)-[1,1′-biphenyl]-3-yl)-3-(((S)-2-(hydroxymethyl)pyrrolidin-1-yl)methyl)-4H-pyrido[1,2-a]pyrimidin-4-one;
8-(2,2′-dichloro-3′-(6-methoxy-5-(((((S)-5-oxopyrrolidin-2-yl)methyl)amino)methyl)pyridin-2-yl)-[1,1′-biphenyl]-3-yl)-3-(((R)-2-(hydroxymethyl)pyrrolidin-1-yl)methyl)-4H-pyrido[1,2-a]pyrimidin-4-one;
8-(2,2′-dichloro-3′-(6-methoxy-5-(((((R)-5-oxopyrrolidin-2-yl)methyl)amino)methyl)pyridin-2-yl)-[1,1′-biphenyl]-3-yl)-3-(((R)-2-(hydroxymethyl)pyrrolidin-1-yl)methyl)-4H-pyrido[1,2-a]pyrimidin-4-one;
(S)-3-(((8-(2,2′-dichloro-3′-(6-methoxy-5-((((5-oxopyrrolidin-2-yl)methyl)amino)methyl)pyridin-2-yl)-[1,1′-biphenyl]-3-yl)-4-oxo-4H-pyrido[1,2-a]pyrimidin-3-yl)methyl)amino)-3-methylbutanoic acid;
(R)-3-(((8-(2,2′-dichloro-3′-(6-methoxy-5-((((5-oxopyrrolidin-2-yl)methyl)amino)methyl)pyridin-2-yl)-[1,1′-biphenyl]-3-yl)-4-oxo-4H-pyrido[1,2-a]pyrimidin-3-yl)methyl)amino)-3-methylbutanoic acid;
(S)-5-((6-(2,2′-dichloro-3′-(6-methoxy-5-(((((S)-5-oxopyrrolidin-2-yl)methyl)amino)methyl)pyridin-2-yl)-[1,1′-biphenyl]-3-yl)-8-methoxy-3,4-dihydroisoquinolin-2(1H)-yl)methyl)pyrrolidin-2-one;
(R)-5-((6-(2,2′-dichloro-3′-(6-methoxy-5-(((((R)-5-oxopyrrolidin-2-yl)methyl)amino)methyl)pyridin-2-yl)-[1,1′-biphenyl]-3-yl)-8-methoxy-3,4-dihydroisoquinolin-2(1H)-yl)methyl)pyrrolidin-2-one; 5-((6-(2,2′-dichloro-3′-(R)-(6-methoxy-5-(((((S)-5-oxopyrrolidin-2-yl)methyl)amino)methyl)pyridin-2-yl)-[1,1′-biphenyl]-3-yl)-8-methoxy-3,4-dihydroisoquinolin-2(1H)-yl)methyl)pyrrolidin-2-one;
(S)-5-((6-(2,2′-dichloro-3′-(6-methoxy-5-(((((R)-5-oxopyrrolidin-2-yl)methyl)amino)methyl)pyridin-2-yl)-[1,1′-biphenyl]-3-yl)-8-methoxy-3,4-dihydroisoquinolin-2(1H)-yl)methyl)pyrrolidin-2-one;
8-(2,2′-dichloro-3′-(6-methoxy-5-(((((S)-5-oxopyrrolidin-2-yl)methyl)amino)methyl)pyridin-2-yl)-[1,1′-biphenyl]-3-yl)-2-(((((S)-5-oxopyrrolidin-2-yl)methyl)amino)methyl)-4H-pyrido[1,2-a]pyrimidin-4-one;
8-(2,2′-dichloro-3′-(6-methoxy-5-(((((R)-5-oxopyrrolidin-2-yl)methyl)amino)methyl)pyridin-2-yl)-[1,1′-biphenyl]-3-yl)-2-(((((S)-5-oxopyrrolidin-2-yl)methyl)amino)methyl)-4H-pyrido[1,2-a]pyrimidin-4-one;
8-(2,2′-dichloro-3′-(6-methoxy-5-(((((S)-5-oxopyrrolidin-2-yl)methyl)amino)methyl)pyridin-2-yl)-[1,1′-biphenyl]-3-yl)-2-(((((R)-5-oxopyrrolidin-2-yl)methyl)amino)methyl)-4H-pyrido[1,2-a]pyrimidin-4-one;
8-(2,2′-dichloro-3′-(6-methoxy-5-(((((R)-5-oxopyrrolidin-2-yl)methyl)amino)methyl)pyridin-2-yl)-[1,1′-biphenyl]-3-yl)-2-(((((R)-5-oxopyrrolidin-2-yl)methyl)amino)methyl)-4H-pyrido[1,2-a]pyrimidin-4-one;
(S)-8-(2,2′-dichloro-3′-(5-((3-hydroxy-3-methylazetidin-1-yl)methyl)-6-methoxypyridin-2-yl)-[1,1′-biphenyl]-3-yl)-3-((((5-oxopyrrolidin-2-yl)methyl)amino)methyl)-4H-pyrido[1,2-a]pyrimidin-4-one;
(R)-8-(2,2′-dichloro-3′-(5-((3-hydroxy-3-methylazetidin-1-yl)methyl)-6-methoxypyridin-2-yl)-[1,1′-biphenyl]-3-yl)-3-((((5-oxopyrrolidin-2-yl)methyl)amino)methyl)-4H-pyrido[1,2-a]pyrimidin-4-one;
8-(2,2′-dichloro-3′-(5-((((1r,3r)-3-hydroxycyclobutyl)amino)methyl)-6-methoxypyridin-2-yl)-[1,1′-biphenyl]-3-yl)-3-(((((S)-5-oxopyrrolidin-2-yl)methyl)amino)methyl)-4H-pyrido[1,2-a]pyrimidin-4-one;
8-(2,2′-dichloro-3′-(5-((((1s,3s)-3-hydroxycyclobutyl)amino)methyl)-6-methoxypyridin-2-yl)-[1,1′-biphenyl]-3-yl)-3-(((((S)-5-oxopyrrolidin-2-yl)methyl)amino)methyl)-4H-pyrido[1,2-a]pyrimidin-4-one;
8-(2,2′-dichloro-3′-(5-((((1r,3r)-3-hydroxycyclobutyl)amino)methyl)-6-methoxypyridin-2-yl)-[1,1′-biphenyl]-3-yl)-3-(((((R)-5-oxopyrrolidin-2-yl)methyl)amino)methyl)-4H-pyrido[1,2-a]pyrimidin-4-one;
8-(2,2′-dichloro-3′-(5-((((1s,3s)-3-hydroxycyclobutyl)amino)methyl)-6-methoxypyridin-2-yl)-[1,1′-biphenyl]-3-yl)-3-(((((R)-5-oxopyrrolidin-2-yl)methyl)amino)methyl)-4H-pyrido[1,2-a]pyrimidin-4-one;
(S)-8-(3′-(5-((3,3-bis(hydroxymethyl)azetidin-1-yl)methyl)-6-methoxypyridin-2-yl)-2,2′-dichloro-[1,1′-biphenyl]-3-yl)-3-((((5-oxopyrrolidin-2-yl)methyl)amino)methyl)-4H-pyrido[1,2-a]pyrimidin-4-one;
(R)-8-(3′-(5-((3,3-bis(hydroxymethyl)azetidin-1-yl)methyl)-6-methoxypyridin-2-yl)-2,2′-dichloro-[1,1′-biphenyl]-3-yl)-3-((((5-oxopyrrolidin-2-yl)methyl)amino)methyl)-4H-pyrido[1,2-a]pyrimidin-4-one;
(S)-4-(((8-(2,2′-dichloro-3′-(6-methoxy-5-((((5-oxopyrrolidin-2-yl)methyl)amino)methyl)pyridin-2-yl)-[1,1′-biphenyl]-3-yl)-4-oxo-4H-pyrido[1,2-a]pyrimidin-3-yl)methyl)amino)butanoic acid;
(R)-4-(((8-(2,2′-dichloro-3′-(6-methoxy-5-((((5-oxopyrrolidin-2-yl)methyl)amino)methyl)pyridin-2-yl)-[1,1′-biphenyl]-3-yl)-4-oxo-4H-pyrido[1,2-a]pyrimidin-3-yl)methyl)amino)butanoic acid;
8-(2,2′-dichloro-3′-(6-methoxy-5-(((((S)-5-oxopyrrolidin-2-yl)methyl)amino)methyl)pyridin-2-yl)-[1,1′-biphenyl]-3-yl)-3-(5-hydroxy-1,4,5,6-tetrahydropyrimidin-2-yl)-4H-pyrido[1,2-a]pyrimidin-4-one;
8-(2,2′-dichloro-3′-(6-methoxy-5-(((((R)-5-oxopyrrolidin-2-yl)methyl)amino)methyl)pyridin-2-yl)-[1,1′-biphenyl]-3-yl)-3-(5-hydroxy-1,4,5,6-tetrahydropyrimidin-2-yl)-4H-pyrido[1,2-a]pyrimidin-4-one;
methyl (S)-1-((8-(2,2′-dichloro-3′-(6-methoxy-5-((((5-oxopyrrolidin-2-yl)methyl)amino)methyl)pyridin-2-yl)-[1,1′-biphenyl]-3-yl)-4-oxo-4H-pyrido[1,2-a]pyrimidin-3-yl)methyl)azetidine-3-carboxylate;
methyl (R)-1-((8-(2,2′-dichloro-3′-(6-methoxy-5-((((5-oxopyrrolidin-2-yl)methyl)amino)methyl)pyridin-2-yl)-[1,1′-biphenyl]-3-yl)-4-oxo-4H-pyrido[1,2-a]pyrimidin-3-yl)methyl)azetidine-3-carboxylate;
8-(2,2′-dichloro-3′-(6-methoxy-5-(((((S)-5-oxopyrrolidin-2-yl)methyl)amino)methyl)pyridin-2-yl)-[1,1′-biphenyl]-3-yl)-3-((((3R,4S)-3-hydroxytetrahydro-2H-pyran-4-yl)amino)methyl)-4H-pyrido[1,2-a]pyrimidin-4-one;
8-(2,2′-dichloro-3′-(6-methoxy-5-(((((S)-5-oxopyrrolidin-2-yl)methyl)amino)methyl)pyridin-2-yl)-[1,1′-biphenyl]-3-yl)-3-((((3S,4S)-3-hydroxytetrahydro-2H-pyran-4-yl)amino)methyl)-4H-pyrido[1,2-a]pyrimidin-4-one;
8-(2,2′-dichloro-3′-(6-methoxy-5-(((((S)-5-oxopyrrolidin-2-yl)methyl)amino)methyl)pyridin-2-yl)-[1,1′-biphenyl]-3-yl)-3-((((3R,4R)-3-hydroxytetrahydro-2H-pyran-4-yl)amino)methyl)-4H-pyrido[1,2-a]pyrimidin-4-one;
8-(2,2′-dichloro-3′-(6-methoxy-5-(((((S)-5-oxopyrrolidin-2-yl)methyl)amino)methyl)pyridin-2-yl)-[1,1′-biphenyl]-3-yl)-3-((((3S,4R)-3-hydroxytetrahydro-2H-pyran-4-yl)amino)methyl)-4H-pyrido[1,2-a]pyrimidin-4-one;
8-(2,2′-dichloro-3′-(6-methoxy-5-(((((R)-5-oxopyrrolidin-2-yl)methyl)amino)methyl)pyridin-2-yl)-[1,1′-biphenyl]-3-yl)-3-((((3R,4S)-3-hydroxytetrahydro-2H-pyran-4-yl)amino)methyl)-4H-pyrido[1,2-a]pyrimidin-4-one;
8-(2,2′-dichloro-3′-(6-methoxy-5-(((((R)-5-oxopyrrolidin-2-yl)methyl)amino)methyl)pyridin-2-yl)-[1,1′-biphenyl]-3-yl)-3-((((3S,4S)-3-hydroxytetrahydro-2H-pyran-4-yl)amino)methyl)-4H-pyrido[1,2-a]pyrimidin-4-one;
8-(2,2′-dichloro-3′-(6-methoxy-5-(((((R)-5-oxopyrrolidin-2-yl)methyl)amino)methyl)pyridin-2-yl)-[1,1′-biphenyl]-3-yl)-3-((((3R,4R)-3-hydroxytetrahydro-2H-pyran-4-yl)amino)methyl)-4H-pyrido[1,2-a]pyrimidin-4-one;
8-(2,2′-dichloro-3′-(6-methoxy-5-(((((R)-5-oxopyrrolidin-2-yl)methyl)amino)methyl)pyridin-2-yl)-[1,1′-biphenyl]-3-yl)-3-((((3S,4R)-3-hydroxytetrahydro-2H-pyran-4-yl)amino)methyl)-4H-pyrido[1,2-a]pyrimidin-4-one;
8-(2,2′-dichloro-3′-(6-methoxy-5-(((((S)-5-oxopyrrolidin-2-yl)methyl)amino)methyl)pyridin-2-yl)-[1,1′-biphenyl]-3-yl)-2-methyl-3-(((((S)-5-oxopyrrolidin-2-yl)methyl)amino)methyl)-4H-pyrido[1,2-a]pyrimidin-4-one;
8-(2,2′-dichloro-3′-(6-methoxy-5-(((((R)-5-oxopyrrolidin-2-yl)methyl)amino)methyl)pyridin-2-yl)-[1,1′-biphenyl]-3-yl)-2-methyl-3-(((((S)-5-oxopyrrolidin-2-yl)methyl)amino)methyl)-4H-pyrido[1,2-a]pyrimidin-4-one;
8-(2,2′-dichloro-3′-(6-methoxy-5-(((((S)-5-oxopyrrolidin-2-yl)methyl)amino)methyl)pyridin-2-yl)-[1,1′-biphenyl]-3-yl)-2-methyl-3-(((((R)-5-oxopyrrolidin-2-yl)methyl)amino)methyl)-4H-pyrido[1,2-a]pyrimidin-4-one;
8-(2,2′-dichloro-3′-(6-methoxy-5-(((((R)-5-oxopyrrolidin-2-yl)methyl)amino)methyl)pyridin-2-yl)-[1,1′-biphenyl]-3-yl)-2-methyl-3-(((((R)-5-oxopyrrolidin-2-yl)methyl)amino)methyl)-4H-pyrido[1,2-a]pyrimidin-4-one;
8-(2,2′-dichloro-3′-(5-(((S)-3-hydroxypyrrolidin-1-yl)methyl)-6-methoxypyridin-2-yl)-[1,1′-biphenyl]-3-yl)-3-(((S)-3-hydroxypyrrolidin-1-yl)methyl)-4H-pyrido[1,2-a]pyrimidin-4-one;
8-(2,2′-dichloro-3′-(5-(((R)-3-hydroxypyrrolidin-1-yl)methyl)-6-methoxypyridin-2-yl)-[1,1′-biphenyl]-3-yl)-3-(((S)-3-hydroxypyrrolidin-1-yl)methyl)-4H-pyrido[1,2-a]pyrimidin-4-one;
8-(2,2′-dichloro-3′-(5-(((S)-3-hydroxypyrrolidin-1-yl)methyl)-6-methoxypyridin-2-yl)-[1,1′-biphenyl]-3-yl)-3-(((R)-3-hydroxypyrrolidin-1-yl)methyl)-4H-pyrido[1,2-a]pyrimidin-4-one;
8-(2,2′-dichloro-3′-(5-(((R)-3-hydroxypyrrolidin-1-yl)methyl)-6-methoxypyridin-2-yl)-[1,1′-biphenyl]-3-yl)-3-(((R)-3-hydroxypyrrolidin-1-yl)methyl)-4H-pyrido[1,2-a]pyrimidin-4-one;
(2R,4R)-1-((8-(2,2′-dichloro-3′-(6-methoxy-5-(((((S)-5-oxopyrrolidin-2-yl)methyl)amino)methyl)pyridin-2-yl)-[1,1′-biphenyl]-3-yl)-4-oxo-4H-pyrido[1,2-a]pyrimidin-3-yl)methyl)-4-hydroxypyrrolidine-2-carboxylic acid;
(2S,4R)-1-((8-(2,2′-dichloro-3′-(6-methoxy-5-(((((S)-5-oxopyrrolidin-2-yl)methyl)amino)methyl)pyridin-2-yl)-[1,1′-biphenyl]-3-yl)-4-oxo-4H-pyrido[1,2-a]pyrimidin-3-yl)methyl)-4-hydroxypyrrolidine-2-carboxylic acid;
(2R,4S)-1-((8-(2,2′-dichloro-3′-(6-methoxy-5-(((((S)-5-oxopyrrolidin-2-yl)methyl)amino)methyl)pyridin-2-yl)-[1,1′-biphenyl]-3-yl)-4-oxo-4H-pyrido[1,2-a]pyrimidin-3-yl)methyl)-4-hydroxypyrrolidine-2-carboxylic acid;
(2S,4SR)-1-((8-(2,2′-dichloro-3′-(6-methoxy-5-(((((S)-5-oxopyrrolidin-2-yl)methyl)amino)methyl)pyridin-2-yl)-[1,1′-biphenyl]-3-yl)-4-oxo-4H-pyrido[1,2-a]pyrimidin-3-yl)methyl)-4-hydroxypyrrolidine-2-carboxylic acid;
(2R,4R)-1-((8-(2,2′-dichloro-3′-(6-methoxy-5-(((((R)-5-oxopyrrolidin-2-yl)methyl)amino)methyl)pyridin-2-yl)-[1,1′-biphenyl]-3-yl)-4-oxo-4H-pyrido[1,2-a]pyrimidin-3-yl)methyl)-4-hydroxypyrrolidine-2-carboxylic acid;
(2S,4R)-1-((8-(2,2′-dichloro-3′-(6-methoxy-5-(((((R)-5-oxopyrrolidin-2-yl)methyl)amino)methyl)pyridin-2-yl)-[1,1′-biphenyl]-3-yl)-4-oxo-4H-pyrido[1,2-a]pyrimidin-3-yl)methyl)-4-hydroxypyrrolidine-2-carboxylic acid;
(2R,4S)-1-((8-(2,2′-dichloro-3′-(6-methoxy-5-(((((R)-5-oxopyrrolidin-2-yl)methyl)amino)methyl)pyridin-2-yl)-[1,1′-biphenyl]-3-yl)-4-oxo-4H-pyrido[1,2-a]pyrimidin-3-yl)methyl)-4-hydroxypyrrolidine-2-carboxylic acid;
(2S,4SR)-1-((8-(2,2′-dichloro-3′-(6-methoxy-5-(((((R)-5-oxopyrrolidin-2-yl)methyl)amino)methyl)pyridin-2-yl)-[1,1′-biphenyl]-3-yl)-4-oxo-4H-pyrido[1,2-a]pyrimidin-3-yl)methyl)-4-hydroxypyrrolidine-2-carboxylic acid;
methyl (S)-2-(8-(2,2′-dichloro-3′-(6-methoxy-5-((((5-oxopyrrolidin-2-yl)methyl)amino)methyl)pyridin-2-yl)-[1,1′-biphenyl]-3-yl)-4-oxo-4H-pyrido[1,2-a]pyrimidin-3-yl)-1H-imidazole-5-carboxylate;
methyl (R)-2-(8-(2,2′-dichloro-3′-(6-methoxy-5-((((5-oxopyrrolidin-2-yl)methyl)amino)methyl)pyridin-2-yl)-[1,1′-biphenyl]-3-yl)-4-oxo-4H-pyrido[1,2-a]pyrimidin-3-yl)-1H-imidazole-5-carboxylate;
(S)-1-((8-(2,2′-dichloro-3′-(5-(((2-hydroxyethyl)amino)methyl)-6-methoxypyridin-2-yl)-[1,1′-biphenyl]-3-yl)-4-oxo-4H-pyrido[1,2-a]pyrimidin-3-yl)methyl)-2-methylpyrrolidine-2-carboxylic acid;
(R)-1-((8-(2,2′-dichloro-3′-(5-(((2-hydroxyethyl)amino)methyl)-6-methoxypyridin-2-yl)-[1,1′-biphenyl]-3-yl)-4-oxo-4H-pyrido[1,2-a]pyrimidin-3-yl)methyl)-2-methylpyrrolidine-2-carboxylic acid;
8-(2,2′-dichloro-3′-(6-methoxy-5-(((((S)-5-oxopyrrolidin-2-yl)methyl)amino)methyl)pyridin-2-yl)-[1,1′-biphenyl]-3-yl)-3-(((R)-3-hydroxypyrrolidin-1-yl)methyl)-4H-pyrido[1,2-a]pyrimidin-4-one;
8-(2,2′-dichloro-3′-(6-methoxy-5-(((((R)-5-oxopyrrolidin-2-yl)methyl)amino)methyl)pyridin-2-yl)-[1,1′-biphenyl]-3-yl)-3-(((R)-3-hydroxypyrrolidin-1-yl)methyl)-4H-pyrido[1,2-a]pyrimidin-4-one;
8-(2,2′-dichloro-3′-(6-methoxy-5-(((((S)-5-oxopyrrolidin-2-yl)methyl)amino)methyl)pyridin-2-yl)-[1,1′-biphenyl]-3-yl)-3-(((S)-3-hydroxypyrrolidin-1-yl)methyl)-4H-pyrido[1,2-a]pyrimidin-4-one;
8-(2,2′-dichloro-3′-(6-methoxy-5-(((((R)-5-oxopyrrolidin-2-yl)methyl)amino)methyl)pyridin-2-yl)-[1,1′-biphenyl]-3-yl)-3-(((S)-3-hydroxypyrrolidin-1-yl)methyl)-4H-pyrido[1,2-a]pyrimidin-4-one;
8-(2,2′-dichloro-3′-(5-(((R)-3-hydroxypyrrolidin-1-yl)methyl)-6-methoxypyridin-2-yl)-[1,1′-biphenyl]-3-yl)-3-(((((S)-5-oxopyrrolidin-2-yl)methyl)amino)methyl)-4H-pyrido[1,2-a]pyrimidin-4-one;
8-(2,2′-dichloro-3′-(5-(((S)-3-hydroxypyrrolidin-1-yl)methyl)-6-methoxypyridin-2-yl)-[1,1′-biphenyl]-3-yl)-3-(((((S)-5-oxopyrrolidin-2-yl)methyl)amino)methyl)-4H-pyrido[1,2-a]pyrimidin-4-one;
8-(2,2′-dichloro-3′-(5-(((R)-3-hydroxypyrrolidin-1-yl)methyl)-6-methoxypyridin-2-yl)-[1,1′-biphenyl]-3-yl)-3-(((((R)-5-oxopyrrolidin-2-yl)methyl)amino)methyl)-4H-pyrido[1,2-a]pyrimidin-4-one;
8-(2,2′-dichloro-3′-(5-(((S)-3-hydroxypyrrolidin-1-yl)methyl)-6-methoxypyridin-2-yl)-[1,1′-biphenyl]-3-yl)-3-(((((R)-5-oxopyrrolidin-2-yl)methyl)amino)methyl)-4H-pyrido[1,2-a]pyrimidin-4-one;
methyl (S)-2-(8-(2,2′-dichloro-3′-(6-methoxy-5-(((((S)-5-oxopyrrolidin-2-yl)methyl)amino)methyl)pyridin-2-yl)-[1,1′-biphenyl]-3-yl)-4-oxo-4H-pyrido[1,2-a]pyrimidin-3-yl)-4,5-dihydro-1H-imidazole-5-carboxylate;
methyl (R)-2-(8-(2,2′-dichloro-3′-(6-methoxy-5-(((((S)-5-oxopyrrolidin-2-yl)methyl)amino)methyl)pyridin-2-yl)-[1,1′-biphenyl]-3-yl)-4-oxo-4H-pyrido[1,2-a]pyrimidin-3-yl)-4,5-dihydro-1H-imidazole-5-carboxylate;
methyl (S)-2-(8-(2,2′-dichloro-3′-(6-methoxy-5-(((((R)-5-oxopyrrolidin-2-yl)methyl)amino)methyl)pyridin-2-yl)-[1,1′-biphenyl]-3-yl)-4-oxo-4H-pyrido[1,2-a]pyrimidin-3-yl)-4,5-dihydro-1H-imidazole-5-carboxylate;
methyl (R)-2-(8-(2,2′-dichloro-3′-(6-methoxy-5-(((((R)-5-oxopyrrolidin-2-yl)methyl)amino)methyl)pyridin-2-yl)-[1,1′-biphenyl]-3-yl)-4-oxo-4H-pyrido[1,2-a]pyrimidin-3-yl)-4,5-dihydro-1H-imidazole-5-carboxylate;
(S)-1-((8-(2,2′-dichloro-3′-(5-(((2-hydroxyethyl)amino)methyl)-6-methoxypyridin-2-yl)-[1,1′-biphenyl]-3-yl)-4-oxo-4H-pyrido[1,2-a]pyrimidin-3-yl)methyl)pyrrolidine-3-carboxylic acid;
(R)-1-((8-(2,2′-dichloro-3′-(5-(((2-hydroxyethyl)amino)methyl)-6-methoxypyridin-2-yl)-[1,1′-biphenyl]-3-yl)-4-oxo-4H-pyrido[1,2-a]pyrimidin-3-yl)methyl)pyrrolidine-3-carboxylic
20 acid;
(S)-1-(5-chloro-2-(2-cyanoethoxy)-4-((E)-2-(2,2′-dichloro-3′-(4-oxo-3-(((((S)-5-oxopyrrolidin-2-yl)methyl)amino)methyl)-4H-pyrido[1,2-a]pyrimidin-8-yl)-[1,1′-biphenyl]-3-yl)vinyl)benzyl)pyrrolidine-3-carboxylic acid;
(R)-1-(5-chloro-2-(2-cyanoethoxy)-4-((E)-2-(2,2′-dichloro-3′-(4-oxo-3-(((((S)-5-oxopyrrolidin-2-yl)methyl)amino)methyl)-4H-pyrido[1,2-a]pyrimidin-8-yl)-[1,1′-biphenyl]-3-yl)vinyl)benzyl)pyrrolidine-3-carboxylic acid;
(S)-1-(5-chloro-2-(2-cyanoethoxy)-4-((E)-2-(2,2′-dichloro-3′-(4-oxo-3-(((((R)-5-oxopyrrolidin-2-yl)methyl)amino)methyl)-4H-pyrido[1,2-a]pyrimidin-8-yl)-[1,1′-biphenyl]-3-yl)vinyl)benzyl)pyrrolidine-3-carboxylic acid;
(R)-1-(5-chloro-2-(2-cyanoethoxy)-4-((E)-2-(2,2′-dichloro-3′-(4-oxo-3-(((((R)-5-oxopyrrolidin-2-yl)methyl)amino)methyl)-4H-pyrido[1,2-a]pyrimidin-8-yl)-[1,1′-biphenyl]-3-yl)vinyl)benzyl)pyrrolidine-3-carboxylic acid;
(S)-1-(5-chloro-2-(2-cyanoethoxy)-4-((Z)-2-(2,2′-dichloro-3′-(4-oxo-3-(((((S)-5-oxopyrrolidin-2-yl)methyl)amino)methyl)-4H-pyrido[1,2-a]pyrimidin-8-yl)-[1,1′-biphenyl]-3-yl)vinyl)benzyl)pyrrolidine-3-carboxylic acid;
(R)-1-(5-chloro-2-(2-cyanoethoxy)-4-((Z)-2-(2,2′-dichloro-3′-(4-oxo-3-(((((S)-5-oxopyrrolidin-2-yl)methyl)amino)methyl)-4H-pyrido[1,2-a]pyrimidin-8-yl)-[1,1′-biphenyl]-3-yl)vinyl)benzyl)pyrrolidine-3-carboxylic acid;
(S)-1-(5-chloro-2-(2-cyanoethoxy)-4-((Z)-2-(2,2′-dichloro-3′-(4-oxo-3-(((((R)-5-oxopyrrolidin-2-yl)methyl)amino)methyl)-4H-pyrido[1,2-a]pyrimidin-8-yl)-[1,1′-biphenyl]-3-yl)vinyl)benzyl)pyrrolidine-3-carboxylic acid;
(R)-1-(5-chloro-2-(2-cyanoethoxy)-4-((Z)-2-(2,2′-dichloro-3′-(4-oxo-3-(((((R)-5-oxopyrrolidin-2-yl)methyl)amino)methyl)-4H-pyrido[1,2-a]pyrimidin-8-yl)-[1,1′-biphenyl]-3-yl)vinyl)benzyl)pyrrolidine-3-carboxylic acid;
8-(2,2′-dichloro-3′-(5-((((3R,4S)-3-hydroxytetrahydro-2H-pyran-4-yl)amino)methyl)-6-methoxypyridin-2-yl)-[1,1′-biphenyl]-3-yl)-3-(((((S)-5-oxopyrrolidin-2-yl)methyl)amino)methyl)-4H-pyrido[1,2-a]pyrimidin-4-one;
8-(2,2′-dichloro-3′-(5-((((3R,4R)-3-hydroxytetrahydro-2H-pyran-4-yl)amino)methyl)-6-methoxypyridin-2-yl)-[1,1′-biphenyl]-3-yl)-3-(((((S)-5-oxopyrrolidin-2-yl)methyl)amino)methyl)-4H-pyrido[1,2-a]pyrimidin-4-one;
8-(2,2′-dichloro-3′-(5-((((3S,4S)-3-hydroxytetrahydro-2H-pyran-4-yl)amino)methyl)-6-methoxypyridin-2-yl)-[1,1′-biphenyl]-3-yl)-3-(((((S)-5-oxopyrrolidin-2-yl)methyl)amino)methyl)-4H-pyrido[1,2-a]pyrimidin-4-one;
8-(2,2′-dichloro-3′-(5-((((3S,4R)-3-hydroxytetrahydro-2H-pyran-4-yl)amino)methyl)-6-methoxypyridin-2-yl)-[1,1′-biphenyl]-3-yl)-3-(((((S)-5-oxopyrrolidin-2-yl)methyl)amino)methyl)-4H-pyrido[1,2-a]pyrimidin-4-one;
8-(2,2′-dichloro-3′-(5-((((3R,4S)-3-hydroxytetrahydro-2H-pyran-4-yl)amino)methyl)-6-methoxypyridin-2-yl)-[1,1′-biphenyl]-3-yl)-3-(((((R)-5-oxopyrrolidin-2-yl)methyl)amino)methyl)-4H-pyrido[1,2-a]pyrimidin-4-one;
8-(2,2′-dichloro-3′-(5-((((3R,4R)-3-hydroxytetrahydro-2H-pyran-4-yl)amino)methyl)-6-methoxypyridin-2-yl)-[1,1′-biphenyl]-3-yl)-3-(((((R)-5-oxopyrrolidin-2-yl)methyl)amino)methyl)-4H-pyrido[1,2-a]pyrimidin-4-one;
8-(2,2′-dichloro-3′-(5-((((3S,4S)-3-hydroxytetrahydro-2H-pyran-4-yl)amino)methyl)-6-methoxypyridin-2-yl)-[1,1′-biphenyl]-3-yl)-3-(((((R)-5-oxopyrrolidin-2-yl)methyl)amino)methyl)-4H-pyrido[1,2-a]pyrimidin-4-one;
8-(2,2′-dichloro-3′-(5-((((3S,4R)-3-hydroxytetrahydro-2H-pyran-4-yl)amino)methyl)-6-methoxypyridin-2-yl)-[1,1′-biphenyl]-3-yl)-3-(((((R)-5-oxopyrrolidin-2-yl)methyl)amino)methyl)-4H-pyrido[1,2-a]pyrimidin-4-one;
8-(2,2′-dichloro-3′-(5-(((S)-3-hydroxypyrrolidin-1-yl)methyl)-6-methoxypyridin-2-yl)-[1,1′-biphenyl]-3-yl)-3-(((((S)-5-oxopyrrolidin-2-yl)methyl)amino)methyl)-4H-pyrido[1,2-a]pyrimidin-4-one;
8-(2,2′-dichloro-3′-(5-(((R)-3-hydroxypyrrolidin-1-yl)methyl)-6-methoxypyridin-2-yl)-[1,1′-biphenyl]-3-yl)-3-(((((S)-5-oxopyrrolidin-2-yl)methyl)amino)methyl)-4H-pyrido[1,2-a]pyrimidin-4-one;
8-(2,2′-dichloro-3′-(5-(((S)-3-hydroxypyrrolidin-1-yl)methyl)-6-methoxypyridin-2-yl)-[1,1′-biphenyl]-3-yl)-3-(((((R)-5-oxopyrrolidin-2-yl)methyl)amino)methyl)-4H-pyrido[1,2-a]pyrimidin-4-one;
8-(2,2′-dichloro-3′-(5-(((R)-3-hydroxypyrrolidin-1-yl)methyl)-6-methoxypyridin-2-yl)-[1,1′-biphenyl]-3-yl)-3-(((((R)-5-oxopyrrolidin-2-yl)methyl)amino)methyl)-4H-pyrido[1,2-a]pyrimidin-4-one;
8-(2,2′-dichloro-3′-(5-methoxy-6-(((((S)-5-oxopyrrolidin-2-yl)methyl)amino)methyl)quinolin-2-yl)-[1,1′-biphenyl]-3-yl)-3-(((((S)-5-oxopyrrolidin-2-yl)methyl)amino)methyl)-4H-pyrido[1,2-a]pyrimidin-4-one;
8-(2,2′-dichloro-3′-(5-methoxy-6-(((((R)-5-oxopyrrolidin-2-yl)methyl)amino)methyl)quinolin-2-yl)-[1,1′-biphenyl]-3-yl)-3-(((((S)-5-oxopyrrolidin-2-yl)methyl)amino)methyl)-4H-pyrido[1,2-a]pyrimidin-4-one;
8-(2,2′-dichloro-3′-(5-methoxy-6-(((((S)-5-oxopyrrolidin-2-yl)methyl)amino)methyl)quinolin-2-yl)-[1,1′-biphenyl]-3-yl)-3-(((((R)-5-oxopyrrolidin-2-yl)methyl)amino)methyl)-4H-pyrido[1,2-a]pyrimidin-4-one;
8-(2,2′-dichloro-3′-(5-methoxy-6-(((((R)-5-oxopyrrolidin-2-yl)methyl)amino)methyl)quinolin-2-yl)-[1,1′-biphenyl]-3-yl)-3-(((((R)-5-oxopyrrolidin-2-yl)methyl)amino)methyl)-4H-pyrido[1,2-a]pyrimidin-4-one;
8-(2-chloro-3-((E)-1-((3-chloro-6-methoxy-5-(((((S)-5-oxopyrrolidin-2-yl)methyl)amino)methyl)pyridin-2-yl)methylene)-2,3-dihydro-1H-inden-4-yl)phenyl)-3-(((((S)-5-oxopyrrolidin-2-yl)methyl)amino)methyl)-4H-pyrido[1,2-a]pyrimidin-4-one;
8-(2-chloro-3-((E)-1-((3-chloro-6-methoxy-5-(((((R)-5-oxopyrrolidin-2-yl)methyl)amino)methyl)pyridin-2-yl)methylene)-2,3-dihydro-1H-inden-4-yl)phenyl)-3-(((((S)-5-oxopyrrolidin-2-yl)methyl)amino)methyl)-4H-pyrido[1,2-a]pyrimidin-4-one;
8-(2-chloro-3-((E)-1-((3-chloro-6-methoxy-5-(((((S)-5-oxopyrrolidin-2-yl)methyl)amino)methyl)pyridin-2-yl)methylene)-2,3-dihydro-1H-inden-4-yl)phenyl)-3-(((((R)-5-oxopyrrolidin-2-yl)methyl)amino)methyl)-4H-pyrido[1,2-a]pyrimidin-4-one;
8-(2-chloro-3-((E)-1-((3-chloro-6-methoxy-5-(((((R)-5-oxopyrrolidin-2-yl)methyl)amino)methyl)pyridin-2-yl)methylene)-2,3-dihydro-1H-inden-4-yl)phenyl)-3-(((((R)-5-oxopyrrolidin-2-yl)methyl)amino)methyl)-4H-pyrido[1,2-a]pyrimidin-4-one;
8-(2-chloro-3-((Z)-1-((3-chloro-6-methoxy-5-(((((S)-5-oxopyrrolidin-2-yl)methyl)amino)methyl)pyridin-2-yl)methylene)-2,3-dihydro-1H-inden-4-yl)phenyl)-3-(((((S)-5-oxopyrrolidin-2-yl)methyl)amino)methyl)-4H-pyrido[1,2-a]pyrimidin-4-one;
8-(2-chloro-3-((Z)-1-((3-chloro-6-methoxy-5-(((((R)-5-oxopyrrolidin-2-yl)methyl)amino)methyl)pyridin-2-yl)methylene)-2,3-dihydro-1H-inden-4-yl)phenyl)-3-(((((S)-5-oxopyrrolidin-2-yl)methyl)amino)methyl)-4H-pyrido[1,2-a]pyrimidin-4-one;
8-(2-chloro-3-((Z)-1-((3-chloro-6-methoxy-5-(((((S)-5-oxopyrrolidin-2-yl)methyl)amino)methyl)pyridin-2-yl)methylene)-2,3-dihydro-1H-inden-4-yl)phenyl)-3-(((((R)-5-oxopyrrolidin-2-yl)methyl)amino)methyl)-4H-pyrido[1,2-a]pyrimidin-4-one;
8-(2-chloro-3-((Z)-1-((3-chloro-6-methoxy-5-(((((R)-5-oxopyrrolidin-2-yl)methyl)amino)methyl)pyridin-2-yl)methylene)-2,3-dihydro-1H-inden-4-yl)phenyl)-3-(((((R)-5-oxopyrrolidin-2-yl)methyl)amino)methyl)-4H-pyrido[1,2-a]pyrimidin-4-one;
8-(2,2′-dichloro-3′-(5-(5-hydroxy-1,4,5,6-tetrahydropyrimidin-2-yl)-6-methoxypyridin-2-yl)-[1,1′-biphenyl]-3-yl)-3-(((((S)-5-oxopyrrolidin-2-yl)methyl)amino)methyl)-4H-pyrido[1,2-a]pyrimidin-4-one;
8-(2,2′-dichloro-3′-(5-(5-hydroxy-1,4,5,6-tetrahydropyrimidin-2-yl)-6-methoxypyridin-2-yl)-[1,1′-biphenyl]-3-yl)-3-(((((R)-5-oxopyrrolidin-2-yl)methyl)amino)methyl)-4H-pyrido[1,2-a]pyrimidin-4-one;
2-(8-(2,2′-dichloro-3′-(6-methoxy-5-(((((S)-5-oxopyrrolidin-2-yl)methyl)amino)methyl)pyridin-2-yl)-[1,1′-biphenyl]-3-yl)-4-oxo-4H-pyrido[1,2-a]pyrimidin-3-yl)-4,5-dihydro-1H-imidazole-(5S)-carboxylic acid;
2-(8-(2,2′-dichloro-3′-(6-methoxy-5-(((((R)-5-oxopyrrolidin-2-yl)methyl)amino)methyl)pyridin-2-yl)-[1,1′-biphenyl]-3-yl)-4-oxo-4H-pyrido[1,2-a]pyrimidin-3-yl)-4,5-dihydro-1H-imidazole-(5S)-carboxylic acid;
2-(8-(2,2′-dichloro-3′-(6-methoxy-5-(((((S)-5-oxopyrrolidin-2-yl)methyl)amino)methyl)pyridin-2-yl)-[1,1′-biphenyl]-3-yl)-4-oxo-4H-pyrido[1,2-a]pyrimidin-3-yl)-4,5-dihydro-1H-imidazole-(5R)-carboxylic acid;
2-(8-(2,2′-dichloro-3′-(6-methoxy-5-(((((R)-5-oxopyrrolidin-2-yl)methyl)amino)methyl)pyridin-2-yl)-[1,1′-biphenyl]-3-yl)-4-oxo-4H-pyrido[1,2-a]pyrimidin-3-yl)-4,5-dihydro-1H-imidazole-(5R)-carboxylic acid;
2-(6-(2,2′-dichloro-3′-(4-oxo-3-(((((S)-5-oxopyrrolidin-2-yl)methyl)amino)methyl)-4H-pyrido[1,2-a]pyrimidin-8-yl)-[1,1′-biphenyl]-3-yl)-2-methoxypyridin-3-yl)-3,4,5,6-tetrahydropyrimidine-(4S)-carboxylic acid;
2-(6-(2,2′-dichloro-3′-(4-oxo-3-(((((R)-5-oxopyrrolidin-2-yl)methyl)amino)methyl)-4H-pyrido[1,2-a]pyrimidin-8-yl)-[1,1′-biphenyl]-3-yl)-2-methoxypyridin-3-yl)-3,4,5,6-tetrahydropyrimidine-(4S)-carboxylic acid;
2-(6-(2,2′-dichloro-3′-(4-oxo-3-(((((S)-5-oxopyrrolidin-2-yl)methyl)amino)methyl)-4H-pyrido[1,2-a]pyrimidin-8-yl)-[1,1′-biphenyl]-3-yl)-2-methoxypyridin-3-yl)-3,4,5,6-tetrahydropyrimidine-(4R)-carboxylic acid;
2-(6-(2,2′-dichloro-3′-(4-oxo-3-(((((R)-5-oxopyrrolidin-2-yl)methyl)amino)methyl)-4H-pyrido[1,2-a]pyrimidin-8-yl)-[1,1′-biphenyl]-3-yl)-2-methoxypyridin-3-yl)-3,4,5,6-tetrahydropyrimidine-(4R)-carboxylic acid;
8-(2,2′-dichloro-3′-(5-((((S)-2-hydroxypropyl)amino)methyl)-6-methoxypyridin-2-yl)-[1,1′-biphenyl]-3-yl)-3-((((S)-2-hydroxypropyl)amino)methyl)-2-methyl-4H-pyrido[1,2-a]pyrimidin-4-one;
8-(2,2′-dichloro-3′-(5-((((R)-2-hydroxypropyl)amino)methyl)-6-methoxypyridin-2-yl)-[1,1′-biphenyl]-3-yl)-3-((((S)-2-hydroxypropyl)amino)methyl)-2-methyl-4H-pyrido[1,2-a]pyrimidin-4-one;
8-(2,2′-dichloro-3′-(5-((((S)-2-hydroxypropyl)amino)methyl)-6-methoxypyridin-2-yl)-[1,1′-biphenyl]-3-yl)-3-((((R)-2-hydroxypropyl)amino)methyl)-2-methyl-4H-pyrido[1,2-a]pyrimidin-4-one;
8-(2,2′-dichloro-3′-(5-((((R)-2-hydroxypropyl)amino)methyl)-6-methoxypyridin-2-yl)-[1,1′-biphenyl]-3-yl)-3-((((R)-2-hydroxypropyl)amino)methyl)-2-methyl-4H-pyrido[1,2-a]pyrimidin-4-one;
(S)-3-(((8-(2,2′-dichloro-3′-(6-methoxy-5-((((5-oxopyrrolidin-2-yl)methyl)amino)methyl)pyridin-2-yl)-[1,1′-biphenyl]-3-yl)-4-oxo-4H-pyrido[1,2-a]pyrimidin-3-yl)methyl)amino)propanoic acid;
(R)-3-(((8-(2,2′-dichloro-3′-(6-methoxy-5-((((5-oxopyrrolidin-2-yl)methyl)amino)methyl)pyridin-2-yl)-[1,1′-biphenyl]-3-yl)-4-oxo-4H-pyrido[1,2-a]pyrimidin-3-yl)methyl)amino)propanoic acid;
8-(2,2′-dichloro-3′-(6-methoxy-5-(((((S)-5-oxopyrrolidin-2-yl)methyl)amino)methyl)pyridin-2-yl)-[1,1′-biphenyl]-3-yl)-6-methyl-3-(((((S)-5-oxopyrrolidin-2-yl)methyl)amino)methyl)-4H-pyrido[1,2-a]pyrimidin-4-one;
8-(2,2′-dichloro-3′-(6-methoxy-5-(((((R)-5-oxopyrrolidin-2-yl)methyl)amino)methyl)pyridin-2-yl)-[1,1′-biphenyl]-3-yl)-6-methyl-3-(((((S)-5-oxopyrrolidin-2-yl)methyl)amino)methyl)-4H-pyrido[1,2-a]pyrimidin-4-one;
8-(2,2′-dichloro-3′-(6-methoxy-5-(((((S)-5-oxopyrrolidin-2-yl)methyl)amino)methyl)pyridin-2-yl)-[1,1′-biphenyl]-3-yl)-6-methyl-3-(((((R)-5-oxopyrrolidin-2-yl)methyl)amino)methyl)-4H-pyrido[1,2-a]pyrimidin-4-one;
8-(2,2′-dichloro-3′-(6-methoxy-5-(((((R)-5-oxopyrrolidin-2-yl)methyl)amino)methyl)pyridin-2-yl)-[1,1′-biphenyl]-3-yl)-6-methyl-3-(((((R)-5-oxopyrrolidin-2-yl)methyl)amino)methyl)-4H-pyrido[1,2-a]pyrimidin-4-one;
(S)-2-(((8-(2,2′-dichloro-3′-(6-methoxy-5-((((5-oxopyrrolidin-2-yl)methyl)amino)methyl)pyridin-2-yl)-[1,1′-biphenyl]-3-yl)-4-oxo-4H-pyrido[1,2-a]pyrimidin-3-yl)methyl)amino)-2-methylpropanoic acid;
(R)-2-(((8-(2,2′-dichloro-3′-(6-methoxy-5-((((5-oxopyrrolidin-2-yl)methyl)amino)methyl)pyridin-2-yl)-[1,1′-biphenyl]-3-yl)-4-oxo-4H-pyrido[1,2-a]pyrimidin-3-yl)methyl)amino)-2-methylpropanoic acid;
8-(2,2′-dichloro-3′-(5-((((R)-2-hydroxypropyl)amino)methyl)-6-methoxypyridin-2-yl)-[1,1′-biphenyl]-3-yl)-3-((((R)-2-hydroxypropyl)amino)methyl)-4H-pyrido[1,2-a]pyrimidin-4-one;
8-(2,2′-dichloro-3′-(5-((((S)-2-hydroxypropyl)amino)methyl)-6-methoxypyridin-2-yl)-[1,1′-biphenyl]-3-yl)-3-((((R)-2-hydroxypropyl)amino)methyl)-4H-pyrido[1,2-a]pyrimidin-4-one;
8-(2,2′-dichloro-3′-(5-((((R)-2-hydroxypropyl)amino)methyl)-6-methoxypyridin-2-yl)-[1,1′-biphenyl]-3-yl)-3-((((S)-2-hydroxypropyl)amino)methyl)-4H-pyrido[1,2-a]pyrimidin-4-one;
8-(2,2′-dichloro-3′-(5-((((S)-2-hydroxypropyl)amino)methyl)-6-methoxypyridin-2-yl)-[1,1′-biphenyl]-3-yl)-3-((((S)-2-hydroxypropyl)amino)methyl)-4H-pyrido[1,2-a]pyrimidin-4-one;
S)-N-((8-(2,2′-dichloro-3′-(6-methoxy-5-((((5-oxopyrrolidin-2-yl)methyl)amino)methyl)pyridin-2-yl)-[1,1′-biphenyl]-3-yl)-4-oxo-4H-pyrido[1,2-a]pyrimidin-3-yl)methyl)-N-methylglycine;
(R)—N-((8-(2,2′-dichloro-3′-(6-methoxy-5-((((5-oxopyrrolidin-2-yl)methyl)amino)methyl)pyridin-2-yl)-[1,1′-biphenyl]-3-yl)-4-oxo-4H-pyrido[1,2-a]pyrimidin-3-yl)methyl)-N-methylglycine;
1-((8-(2,2′-dichloro-3′-(5-(((2-hydroxyethyl)amino)methyl)-6-methoxypyridin-2-yl)-[1,1′-biphenyl]-3-yl)-4-oxo-4H-pyrido[1,2-a]pyrimidin-3-yl)methyl)azetidine-3-carboxylic acid;
(S)-((8-(2,2′-dichloro-3′-(6-methoxy-5-((((5-oxopyrrolidin-2-yl)methyl)amino)methyl)pyridin-2-yl)-[1,1′-biphenyl]-3-yl)-4-oxo-4H-pyrido[1,2-a]pyrimidin-3-yl)methyl)glycine;
(R)-((8-(2,2′-dichloro-3′-(6-methoxy-5-((((5-oxopyrrolidin-2-yl)methyl)amino)methyl)pyridin-2-yl)-[1,1′-biphenyl]-3-yl)-4-oxo-4H-pyrido[1,2-a]pyrimidin-3-yl)methyl)glycine;
N-((8-(2,2′-dichloro-3′-(6-methoxy-5-(((((S)-5-oxopyrrolidin-2-yl)methyl)amino)methyl)pyridin-2-yl)-[1,1′-biphenyl]-3-yl)-4-oxo-4H-pyrido[1,2-a]pyrimidin-3-yl)methyl)-N-methyl-L-alanine;
N-((8-(2,2′-dichloro-3′-(6-methoxy-5-(((((R)-5-oxopyrrolidin-2-yl)methyl)amino)methyl)pyridin-2-yl)-[1,1′-biphenyl]-3-yl)-4-oxo-4H-pyrido[1,2-a]pyrimidin-3-yl)methyl)-N-methyl-L-alanine;
N-((8-(2,2′-dichloro-3′-(6-methoxy-5-(((((S)-5-oxopyrrolidin-2-yl)methyl)amino)methyl)pyridin-2-yl)-[1,1′-biphenyl]-3-yl)-4-oxo-4H-pyrido[1,2-a]pyrimidin-3-yl)methyl)-N-methyl-D-alanine;
N-((8-(2,2′-dichloro-3′-(6-methoxy-5-(((((R)-5-oxopyrrolidin-2-yl)methyl)amino)methyl)pyridin-2-yl)-[1,1′-biphenyl]-3-yl)-4-oxo-4H-pyrido[1,2-a]pyrimidin-3-yl)methyl)-N-methyl-D-alanine;
((8-(2,2′-dichloro-3′-(5-(((2-hydroxyethyl)amino)methyl)-6-methoxypyridin-2-yl)-[1,1′-biphenyl]-3-yl)-4-oxo-4H-pyrido[1,2-a]pyrimidin-3-yl)methyl)-L-serine;
((8-(2,2′-dichloro-3′-(5-(((2-hydroxyethyl)amino)methyl)-6-methoxypyridin-2-yl)-[1,1′-biphenyl]-3-yl)-4-oxo-4H-pyrido[1,2-a]pyrimidin-3-yl)methyl)-D-serine;
methyl (S)-1-((8-(2,2′-dichloro-3′-(6-methoxy-5-(((((S)-5-oxopyrrolidin-2-yl)methyl)amino)methyl)pyridin-2-yl)-[1,1′-biphenyl]-3-yl)-4-oxo-4H-pyrido[1,2-a]pyrimidin-3-yl)methyl)pyrrolidine-3-carboxylate;
methyl (R)-1-((8-(2,2′-dichloro-3′-(6-methoxy-5-(((((S)-5-oxopyrrolidin-2-yl)methyl)amino)methyl)pyridin-2-yl)-[1,1′-biphenyl]-3-yl)-4-oxo-4H-pyrido[1,2-a]pyrimidin-3-yl)methyl)pyrrolidine-3-carboxylate;
methyl (S)-1-((8-(2,2′-dichloro-3′-(6-methoxy-5-(((((R)-5-oxopyrrolidin-2-yl)methyl)amino)methyl)pyridin-2-yl)-[1,1′-biphenyl]-3-yl)-4-oxo-4H-pyrido[1,2-a]pyrimidin-3-yl)methyl)pyrrolidine-3-carboxylate;
methyl (R)-1-((8-(2,2′-dichloro-3′-(6-methoxy-5-(((((R)-5-oxopyrrolidin-2-yl)methyl)amino)methyl)pyridin-2-yl)-[1,1′-biphenyl]-3-yl)-4-oxo-4H-pyrido[1,2-a]pyrimidin-3-yl)methyl)pyrrolidine-3-carboxylate;
8-(2,2′-dichloro-3′-(6-methoxy-5-(((((S)-5-oxopyrrolidin-2-yl)methyl)amino)methyl)pyridin-2-yl)-[1,1′-biphenyl]-3-yl)-3-((((S)-2-hydroxypropyl)amino)methyl)-2-methyl-4H-pyrido[1,2-a]pyrimidin-4-one;
8-(2,2′-dichloro-3′-(6-methoxy-5-(((((R)-5-oxopyrrolidin-2-yl)methyl)amino)methyl)pyridin-2-yl)-[1,1′-biphenyl]-3-yl)-3-((((S)-2-hydroxypropyl)amino)methyl)-2-methyl-4H-pyrido[1,2-a]pyrimidin-4-one;
8-(2,2′-dichloro-3′-(6-methoxy-5-(((((S)-5-oxopyrrolidin-2-yl)methyl)amino)methyl)pyridin-2-yl)-[1,1′-biphenyl]-3-yl)-3-((((R)-2-hydroxypropyl)amino)methyl)-2-methyl-4H-pyrido[1,2-a]pyrimidin-4-one;
8-(2,2′-dichloro-3′-(6-methoxy-5-(((((R)-5-oxopyrrolidin-2-yl)methyl)amino)methyl)pyridin-2-yl)-[1,1′-biphenyl]-3-yl)-3-((((R)-2-hydroxypropyl)amino)methyl)-2-methyl-4H-pyrido[1,2-a]pyrimidin-4-one;
(S)-5-((((8-chloro-2-(2,2′-dichloro-3′-(6-methoxy-5-(((((S)-5-oxopyrrolidin-2-yl)methyl)amino)methyl)pyridin-2-yl)-[1,1′-biphenyl]-3-yl)-5-methoxyquinolin-6-yl)methyl)amino)methyl)pyrrolidin-2-one;
(R)-5-((((8-chloro-2-(2,2′-dichloro-3′-(6-methoxy-5-(((((S)-5-oxopyrrolidin-2-yl)methyl)amino)methyl)pyridin-2-yl)-[1,1′-biphenyl]-3-yl)-5-methoxyquinolin-6-yl)methyl)amino)methyl)pyrrolidin-2-one;
(S)-5-((((8-chloro-2-(2,2′-dichloro-3′-(6-methoxy-5-(((((R)-5-oxopyrrolidin-2-yl)methyl)amino)methyl)pyridin-2-yl)-[1,1′-biphenyl]-3-yl)-5-methoxyquinolin-6-yl)methyl)amino)methyl)pyrrolidin-2-one;
(R)-5-((((8-chloro-2-(2,2′-dichloro-3′-(6-methoxy-5-(((((R)-5-oxopyrrolidin-2-yl)methyl)amino)methyl)pyridin-2-yl)-[1,1′-biphenyl]-3-yl)-5-methoxyquinolin-6-yl)methyl)amino)methyl)pyrrolidin-2-one;
8-(2,2′-dichloro-3′-(5-((((R)-2-hydroxypropyl)amino)methyl)-6-methoxypyridin-2-yl)-[1,1′-biphenyl]-3-yl)-3-((((S)-2-hydroxypropyl)amino)methyl)-4H-pyrido[1,2-a]pyrimidin-4-one;
8-(2,2′-dichloro-3′-(5-((((R)-2-hydroxypropyl)amino)methyl)-6-methoxypyridin-2-yl)-[1,1′-biphenyl]-3-yl)-3-((((R)-2-hydroxypropyl)amino)methyl)-4H-pyrido[1,2-a]pyrimidin-4-one;
8-(2,2′-dichloro-3′-(5-((((S)-2-hydroxypropyl)amino)methyl)-6-methoxypyridin-2-yl)-[1,1′-biphenyl]-3-yl)-3-((((R)-2-hydroxypropyl)amino)methyl)-4H-pyrido[1,2-a]pyrimidin-4-one;
8-(2,2′-dichloro-3′-(5-((((R)-2-hydroxypropyl)amino)methyl)-6-methoxypyridin-2-yl)-[1,1′-biphenyl]-3-yl)-3-((((R)-2-hydroxypropyl)amino)methyl)-4H-pyrido[1,2-a]pyrimidin-4-one;
8-(2,2′-dichloro-3′-(5-((((S)-2-hydroxypropyl)amino)methyl)-6-methoxypyridin-2-yl)-[1,1′-biphenyl]-3-yl)-3-(((((S)-5-oxopyrrolidin-2-yl)methyl)amino)methyl)-4H-pyrido[1,2-a]pyrimidin-4-one;
8-(2,2′-dichloro-3′-(5-((((R)-2-hydroxypropyl)amino)methyl)-6-methoxypyridin-2-yl)-[1,1′-biphenyl]-3-yl)-3-(((((S)-5-oxopyrrolidin-2-yl)methyl)amino)methyl)-4H-pyrido[1,2-a]pyrimidin-4-one;
8-(2,2′-dichloro-3′-(5-((((S)-2-hydroxypropyl)amino)methyl)-6-methoxypyridin-2-yl)-[1,1′-biphenyl]-3-yl)-3-(((((R)-5-oxopyrrolidin-2-yl)methyl)amino)methyl)-4H-pyrido[1,2-a]pyrimidin-4-one;
8-(2,2′-dichloro-3′-(5-((((R)-2-hydroxypropyl)amino)methyl)-6-methoxypyridin-2-yl)-[1,1′-biphenyl]-3-yl)-3-(((((R)-5-oxopyrrolidin-2-yl)methyl)amino)methyl)-4H-pyrido[1,2-a]pyrimidin-4-one;
8-(2,2′-dichloro-3′-(5-((((R)-2-hydroxypropyl)amino)methyl)-6-methoxypyridin-2-yl)-[1,1′-biphenyl]-3-yl)-3-(((((S)-5-oxopyrrolidin-2-yl)methyl)amino)methyl)-4H-pyrido[1,2-a]pyrimidin-4-one;
8-(2,2′-dichloro-3′-(5-((((R)-2-hydroxypropyl)amino)methyl)-6-methoxypyridin-2-yl)-[1,1′-biphenyl]-3-yl)-3-(((((R)-5-oxopyrrolidin-2-yl)methyl)amino)methyl)-4H-pyrido[1,2-a]pyrimidin-4-one;
8-(2,2′-dichloro-3′-(5-((((S)-2-hydroxypropyl)amino)methyl)-6-methoxypyridin-2-yl)-[1,1′-biphenyl]-3-yl)-3-(((((S)-5-oxopyrrolidin-2-yl)methyl)amino)methyl)-4H-pyrido[1,2-a]pyrimidin-4-one;
8-(2,2′-dichloro-3′-(5-((((S)-2-hydroxypropyl)amino)methyl)-6-methoxypyridin-2-yl)-[1,1′-biphenyl]-3-yl)-3-(((((R)-5-oxopyrrolidin-2-yl)methyl)amino)methyl)-4H-pyrido[1,2-a]pyrimidin-4-one;
2-(8-(2,2′-dichloro-3′-(6-methoxy-5-(((((S)-5-oxopyrrolidin-2-yl)methyl)amino)methyl)pyridin-2-yl)-[1,1′-biphenyl]-3-yl)-4-oxo-4H-pyrido[1,2-a]pyrimidin-3-yl)-3,4,5,6-tetrahydropyrimidine-(4S)-carboxylic acid;
2-(8-(2,2′-dichloro-3′-(6-methoxy-5-(((((S)-5-oxopyrrolidin-2-yl)methyl)amino)methyl)pyridin-2-yl)-[1,1′-biphenyl]-3-yl)-4-oxo-4H-pyrido[1,2-a]pyrimidin-3-yl)-3,4,5,6-tetrahydropyrimidine-(4R)-carboxylic acid;
2-(8-(2,2′-dichloro-3′-(6-methoxy-5-(((((R)-5-oxopyrrolidin-2-yl)methyl)amino)methyl)pyridin-2-yl)-[1,1′-biphenyl]-3-yl)-4-oxo-4H-pyrido[1,2-a]pyrimidin-3-yl)-3,4,5,6-tetrahydropyrimidine-(4S)-carboxylic acid;
2-(8-(2,2′-dichloro-3′-(6-methoxy-5-(((((R)-5-oxopyrrolidin-2-yl)methyl)amino)methyl)pyridin-2-yl)-[1,1′-biphenyl]-3-yl)-4-oxo-4H-pyrido[1,2-a]pyrimidin-3-yl)-3,4,5,6-tetrahydropyrimidine-(4R)-carboxylic acid;
(S)-2-(((8-(2,2′-dichloro-3′-(6-methoxy-5-((((5-oxopyrrolidin-2-yl)methyl)amino)methyl)pyridin-2-yl)-[1,1′-biphenyl]-3-yl)-4-oxo-4H-pyrido[1,2-a]pyrimidin-3-yl)methyl)(methyl)amino)-N-methylacetamide;
(R)-2-(((8-(2,2′-dichloro-3′-(6-methoxy-5-((((5-oxopyrrolidin-2-yl)methyl)amino)methyl)pyridin-2-yl)-[1,1′-biphenyl]-3-yl)-4-oxo-4H-pyrido[1,2-a]pyrimidin-3-yl)methyl)(methyl)amino)-N-methylacetamide;
8-(2,2′-dichloro-3′-(6-methoxy-5-(((((S)-5-oxopyrrolidin-2-yl)methyl)amino)methyl)pyridin-2-yl)-[1,1′-biphenyl]-3-yl)-2-methoxy-3-(((((S)-5-oxopyrrolidin-2-yl)methyl)amino)methyl)-4H-pyrido[1,2-a]pyrimidin-4-one;
8-(2,2′-dichloro-3′-(6-methoxy-5-(((((R)-5-oxopyrrolidin-2-yl)methyl)amino)methyl)pyridin-2-yl)-[1,1′-biphenyl]-3-yl)-2-methoxy-3-(((((S)-5-oxopyrrolidin-2-yl)methyl)amino)methyl)-4H-pyrido[1,2-a]pyrimidin-4-one;
8-(2,2′-dichloro-3′-(6-methoxy-5-(((((S)-5-oxopyrrolidin-2-yl)methyl)amino)methyl)pyridin-2-yl)-[1,1′-biphenyl]-3-yl)-2-methoxy-3-(((((R)-5-oxopyrrolidin-2-yl)methyl)amino)methyl)-4H-pyrido[1,2-a]pyrimidin-4-one;
8-(2,2′-dichloro-3′-(6-methoxy-5-(((((R)-5-oxopyrrolidin-2-yl)methyl)amino)methyl)pyridin-2-yl)-[1,1′-biphenyl]-3-yl)-2-methoxy-3-(((((R)-5-oxopyrrolidin-2-yl)methyl)amino)methyl)-4H-pyrido[1,2-a]pyrimidin-4-one;
(S)-2-(((8-(2,2′-dichloro-3′-(6-methoxy-5-((((5-oxopyrrolidin-2-yl)methyl)amino)methyl)pyridin-2-yl)-[1,1′-biphenyl]-3-yl)-4-oxo-4H-pyrido[1,2-a]pyrimidin-3-yl)methyl)amino)-N-methylacetamide;
(R)-2-(((8-(2,2′-dichloro-3′-(6-methoxy-5-((((5-oxopyrrolidin-2-yl)methyl)amino)methyl)pyridin-2-yl)-[1,1′-biphenyl]-3-yl)-4-oxo-4H-pyrido[1,2-a]pyrimidin-3-yl)methyl)amino)-N-methylacetamide;
8-(2,2′-dichloro-3′-(8-chloro-5-methoxy-6-(((((S)-5-oxopyrrolidin-2-yl)methyl)amino)methyl)quinolin-2-yl)-[1,1′-biphenyl]-3-yl)-3-(((((S)-5-oxopyrrolidin-2-yl)methyl)amino)methyl)-4H-pyrido[1,2-a]pyrimidin-4-one;
8-(2,2′-dichloro-3′-(8-chloro-5-methoxy-6-(((((R)-5-oxopyrrolidin-2-yl)methyl)amino)methyl)quinolin-2-yl)-[1,1′-biphenyl]-3-yl)-3-(((((S)-5-oxopyrrolidin-2-yl)methyl)amino)methyl)-4H-pyrido[1,2-a]pyrimidin-4-one;
8-(2,2′-dichloro-3′-(8-chloro-5-methoxy-6-(((((S)-5-oxopyrrolidin-2-yl)methyl)amino)methyl)quinolin-2-yl)-[1,1′-biphenyl]-3-yl)-3-(((((R)-5-oxopyrrolidin-2-yl)methyl)amino)methyl)-4H-pyrido[1,2-a]pyrimidin-4-one;
8-(2,2′-dichloro-3′-(8-chloro-5-methoxy-6-(((((R)-5-oxopyrrolidin-2-yl)methyl)amino)methyl)quinolin-2-yl)-[1,1′-biphenyl]-3-yl)-3-(((((R)-5-oxopyrrolidin-2-yl)methyl)amino)methyl)-4H-pyrido[1,2-a]pyrimidin-4-one;
8-(2,2′-dichloro-3′-(6-methoxy-5-(((((S)-5-oxopyrrolidin-2-yl)methyl)amino)methyl)pyridin-2-yl)-[1,1′-biphenyl]-3-yl)-3-((((1r,3r)-3-hydroxy-3-methylcyclobutyl)amino)methyl)-2-methyl-4H-pyrido[1,2-a]pyrimidin-4-one;
8-(2,2′-dichloro-3′-(6-methoxy-5-(((((S)-5-oxopyrrolidin-2-yl)methyl)amino)methyl)pyridin-2-yl)-[1,1′-biphenyl]-3-yl)-3-((((1s,3s)-3-hydroxy-3-methylcyclobutyl)amino)methyl)-2-methyl-4H-pyrido[1,2-a]pyrimidin-4-one;
8-(2,2′-dichloro-3′-(6-methoxy-5-(((((R)-5-oxopyrrolidin-2-yl)methyl)amino)methyl)pyridin-2-yl)-[1,1′-biphenyl]-3-yl)-3-((((1r,3r)-3-hydroxy-3-methylcyclobutyl)amino)methyl)-2-methyl-4H-pyrido[1,2-a]pyrimidin-4-one;
8-(2,2′-dichloro-3′-(6-methoxy-5-(((((R)-5-oxopyrrolidin-2-yl)methyl)amino)methyl)pyridin-2-yl)-[1,1′-biphenyl]-3-yl)-3-((((1s,3s)-3-hydroxy-3-methylcyclobutyl)amino)methyl)-2-methyl-4H-pyrido[1,2-a]pyrimidin-4-one;
8-(2,2′-dichloro-3′-(5-((((1r,3r)-3-hydroxy-3-methylcyclobutyl)amino)methyl)-6-methoxypyridin-2-yl)-[1,1′-biphenyl]-3-yl)-3-((((1r,3r)-3-hydroxy-3-methylcyclobutyl)amino)methyl)-2-methyl-4H-pyrido[1,2-a]pyrimidin-4-one;
8-(2,2′-dichloro-3′-(5-((((1s,3s)-3-hydroxy-3-methylcyclobutyl)amino)methyl)-6-methoxypyridin-2-yl)-[1,1′-biphenyl]-3-yl)-3-((((1r,3r)-3-hydroxy-3-methylcyclobutyl)amino)methyl)-2-methyl-4H-pyrido[1,2-a]pyrimidin-4-one;
8-(2,2′-dichloro-3′-(5-((((1r,3r)-3-hydroxy-3-methylcyclobutyl)amino)methyl)-6-methoxypyridin-2-yl)-[1,1′-biphenyl]-3-yl)-3-((((1s,3s)-3-hydroxy-3-methylcyclobutyl)amino)methyl)-2-methyl-4H-pyrido[1,2-a]pyrimidin-4-one;
8-(2,2′-dichloro-3′-(5-((((1s,3s)-3-hydroxy-3-methylcyclobutyl)amino)methyl)-6-methoxypyridin-2-yl)-[1,1′-biphenyl]-3-yl)-3-((((1s,3s)-3-hydroxy-3-methylcyclobutyl)amino)methyl)-2-methyl-4H-pyrido[1,2-a]pyrimidin-4-one;
8-(2,2′-dichloro-3′-(6-methoxy-5-(((((S)-5-oxopyrrolidin-2-yl)methyl)amino)methyl)pyridin-2-yl)-[1,1′-biphenyl]-3-yl)-3-(((((6S)-oxopiperidin-2-yl)methyl)amino)methyl)-4H-pyrido[1,2-a]pyrimidin-4-one;
8-(2,2′-dichloro-3′-(6-methoxy-5-(((((R)-5-oxopyrrolidin-2-yl)methyl)amino)methyl)pyridin-2-yl)-[1,1′-biphenyl]-3-yl)-3-(((((6S)-oxopiperidin-2-yl)methyl)amino)methyl)-4H-pyrido[1,2-a]pyrimidin-4-one;
8-(2,2′-dichloro-3′-(6-methoxy-5-(((((S)-5-oxopyrrolidin-2-yl)methyl)amino)methyl)pyridin-2-yl)-[1,1′-biphenyl]-3-yl)-3-(((((6R)-oxopiperidin-2-yl)methyl)amino)methyl)-4H-pyrido[1,2-a]pyrimidin-4-one;
8-(2,2′-dichloro-3′-(6-methoxy-5-(((((R)-5-oxopyrrolidin-2-yl)methyl)amino)methyl)pyridin-2-yl)-[1,1′-biphenyl]-3-yl)-3-(((((6R)-oxopiperidin-2-yl)methyl)amino)methyl)-4H-pyrido[1,2-a]pyrimidin-4-one;
(S)-8-(2,2′-dichloro-3′-(6-methoxy-5-((((5-oxopyrrolidin-2-yl)methyl)amino)methyl)pyridin-2-yl)-[1,1′-biphenyl]-3-yl)-3-(((2-(2-oxoimidazolidin-1-yl)ethyl)amino)methyl)-4H-pyrido[1,2-a]pyrimidin-4-one;
(R)-8-(2,2′-dichloro-3′-(6-methoxy-5-((((5-oxopyrrolidin-2-yl)methyl)amino)methyl)pyridin-2-yl)-[1,1′-biphenyl]-3-yl)-3-(((2-(2-oxoimidazolidin-1-yl)ethyl)amino)methyl)-4H-pyrido[1,2-a]pyrimidin-4-one;
8-(2′-chloro-3′-(6-methoxy-5-(((((S)-5-oxopyrrolidin-2-yl)methyl)amino)methyl)pyridin-2-yl)-2-(trifluoromethyl)-[1,1′-biphenyl]-3-yl)-3-(((((S)-5-oxopyrrolidin-2-yl)methyl)amino)methyl)-4H-pyrido[1,2-a]pyrimidin-4-one;
8-(2′-chloro-3′-(6-methoxy-5-(((((R)-5-oxopyrrolidin-2-yl)methyl)amino)methyl)pyridin-2-yl)-2-(trifluoromethyl)-[1,1′-biphenyl]-3-yl)-3-(((((S)-5-oxopyrrolidin-2-yl)methyl)amino)methyl)-4H-pyrido[1,2-a]pyrimidin-4-one;
8-(2′-chloro-3′-(6-methoxy-5-(((((S)-5-oxopyrrolidin-2-yl)methyl)amino)methyl)pyridin-2-yl)-2-(trifluoromethyl)-[1,1′-biphenyl]-3-yl)-3-(((((R)-5-oxopyrrolidin-2-yl)methyl)amino)methyl)-4H-pyrido[1,2-a]pyrimidin-4-one;
8-(2′-chloro-3′-(6-methoxy-5-(((((R)-5-oxopyrrolidin-2-yl)methyl)amino)methyl)pyridin-2-yl)-2-(trifluoromethyl)-[1,1′-biphenyl]-3-yl)-3-(((((R)-5-oxopyrrolidin-2-yl)methyl)amino)methyl)-4H-pyrido[1,2-a]pyrimidin-4-one;
(S)-2-(((8-(2,2′-dichloro-3′-(6-methoxy-5-((((5-oxopyrrolidin-2-yl)methyl)amino)methyl)pyridin-2-yl)-[1,1′-biphenyl]-3-yl)-4-oxo-4H-pyrido[1,2-a]pyrimidin-3-yl)methyl)amino)-N,2-dimethylpropanamide;
(R)-2-(((8-(2,2′-dichloro-3′-(6-methoxy-5-((((5-oxopyrrolidin-2-yl)methyl)amino)methyl)pyridin-2-yl)-[1,1′-biphenyl]-3-yl)-4-oxo-4H-pyrido[1,2-a]pyrimidin-3-yl)methyl)amino)-N,2-dimethylpropanamide;
(S)-3-(((8-(2,2′-dichloro-3′-(6-methoxy-5-((((5-oxopyrrolidin-2-yl)methyl)amino)methyl)pyridin-2-yl)-[1,1′-biphenyl]-3-yl)-4-oxo-4H-pyrido[1,2-a]pyrimidin-3-yl)methyl)amino)-N-methylpropanamide;
(R)-3-(((8-(2,2′-dichloro-3′-(6-methoxy-5-((((5-oxopyrrolidin-2-yl)methyl)amino)methyl)pyridin-2-yl)-[1,1′-biphenyl]-3-yl)-4-oxo-4H-pyrido[1,2-a]pyrimidin-3-yl)methyl)amino)-N-methylpropanamide;
8-(2,2′-dichloro-3′-(6-methoxy-5-(((((S)-5-oxopyrrolidin-2-yl)methyl)amino)methyl)pyridin-2-yl)-[1,1′-biphenyl]-3-yl)-3-((((1r,3r)-3-hydroxy-3-methylcyclobutyl)amino)methyl)-6-methyl-4H-pyrido[1,2-a]pyrimidin-4-one;
8-(2,2′-dichloro-3′-(6-methoxy-5-(((((S)-5-oxopyrrolidin-2-yl)methyl)amino)methyl)pyridin-2-yl)-[1,1′-biphenyl]-3-yl)-3-((((1s,3s)-3-hydroxy-3-methylcyclobutyl)amino)methyl)-6-methyl-4H-pyrido[1,2-a]pyrimidin-4-one;
8-(2,2′-dichloro-3′-(6-methoxy-5-(((((R)-5-oxopyrrolidin-2-yl)methyl)amino)methyl)pyridin-2-yl)-[1,1′-biphenyl]-3-yl)-3-((((1r,3r)-3-hydroxy-3-methylcyclobutyl)amino)methyl)-6-methyl-4H-pyrido[1,2-a]pyrimidin-4-one;
8-(2,2′-dichloro-3′-(6-methoxy-5-(((((R)-5-oxopyrrolidin-2-yl)methyl)amino)methyl)pyridin-2-yl)-[1,1′-biphenyl]-3-yl)-3-((((1s,3s)-3-hydroxy-3-methylcyclobutyl)amino)methyl)-6-methyl-4H-pyrido[1,2-a]pyrimidin-4-one;
7-(2,2′-dichloro-3′-(6-methoxy-5-(((((S)-5-oxopyrrolidin-2-yl)methyl)amino)methyl)pyridin-2-yl)-[1,1′-biphenyl]-3-yl)-2-(((((S)-5-oxopyrrolidin-2-yl)methyl)amino)methyl)-4H-pyrido[1,2-a]pyrimidin-4-one;
7-(2,2′-dichloro-3′-(6-methoxy-5-(((((R)-5-oxopyrrolidin-2-yl)methyl)amino)methyl)pyridin-2-yl)-[1,1′-biphenyl]-3-yl)-2-(((((S)-5-oxopyrrolidin-2-yl)methyl)amino)methyl)-4H-pyrido[1,2-a]pyrimidin-4-one;
7-(2,2′-dichloro-3′-(6-methoxy-5-(((((S)-5-oxopyrrolidin-2-yl)methyl)amino)methyl)pyridin-2-yl)-[1,1′-biphenyl]-3-yl)-2-(((((R)-5-oxopyrrolidin-2-yl)methyl)amino)methyl)-4H-pyrido[1,2-a]pyrimidin-4-one;
7-(2,2′-dichloro-3′-(6-methoxy-5-(((((R)-5-oxopyrrolidin-2-yl)methyl)amino)methyl)pyridin-2-yl)-[1,1′-biphenyl]-3-yl)-2-(((((R)-5-oxopyrrolidin-2-yl)methyl)amino)methyl)-4H-pyrido[1,2-a]pyrimidin-4-one;
8-(2-chloro-3-((S)-1-((3-chloro-6-methoxy-5-(((((S)-5-oxopyrrolidin-2-yl)methyl)amino)methyl)pyridin-2-yl)methyl)-2,3-dihydro-1H-inden-4-yl)phenyl)-3-(((((S)-5-oxopyrrolidin-2-yl)methyl)amino)methyl)-4H-pyrido[1,2-a]pyrimidin-4-one;
8-(2-chloro-3-((S)-1-((3-chloro-6-methoxy-5-(((((R)-5-oxopyrrolidin-2-yl)methyl)amino)methyl)pyridin-2-yl)methyl)-2,3-dihydro-1H-inden-4-yl)phenyl)-3-(((((S)-5-oxopyrrolidin-2-yl)methyl)amino)methyl)-4H-pyrido[1,2-a]pyrimidin-4-one;
8-(2-chloro-3-((S)-1-((3-chloro-6-methoxy-5-(((((S)-5-oxopyrrolidin-2-yl)methyl)amino)methyl)pyridin-2-yl)methyl)-2,3-dihydro-1H-inden-4-yl)phenyl)-3-(((((R)-5-oxopyrrolidin-2-yl)methyl)amino)methyl)-4H-pyrido[1,2-a]pyrimidin-4-one;
8-(2-chloro-3-((S)-1-((3-chloro-6-methoxy-5-(((((R)-5-oxopyrrolidin-2-yl)methyl)amino)methyl)pyridin-2-yl)methyl)-2,3-dihydro-1H-inden-4-yl)phenyl)-3-(((((R)-5-oxopyrrolidin-2-yl)methyl)amino)methyl)-4H-pyrido[1,2-a]pyrimidin-4-one;
8-(2-chloro-3-((R)-1-((3-chloro-6-methoxy-5-(((((S)-5-oxopyrrolidin-2-yl)methyl)amino)methyl)pyridin-2-yl)methyl)-2,3-dihydro-1H-inden-4-yl)phenyl)-3-(((((S)-5-oxopyrrolidin-2-yl)methyl)amino)methyl)-4H-pyrido[1,2-a]pyrimidin-4-one;
8-(2-chloro-3-((R)-1-((3-chloro-6-methoxy-5-(((((R)-5-oxopyrrolidin-2-yl)methyl)amino)methyl)pyridin-2-yl)methyl)-2,3-dihydro-1H-inden-4-yl)phenyl)-3-(((((S)-5-oxopyrrolidin-2-yl)methyl)amino)methyl)-4H-pyrido[1,2-a]pyrimidin-4-one;
8-(2-chloro-3-((R)-1-((3-chloro-6-methoxy-5-(((((S)-5-oxopyrrolidin-2-yl)methyl)amino)methyl)pyridin-2-yl)methyl)-2,3-dihydro-1H-inden-4-yl)phenyl)-3-(((((R)-5-oxopyrrolidin-2-yl)methyl)amino)methyl)-4H-pyrido[1,2-a]pyrimidin-4-one;
8-(2-chloro-3-((R)-1-((3-chloro-6-methoxy-5-(((((R)-5-oxopyrrolidin-2-yl)methyl)amino)methyl)pyridin-2-yl)methyl)-2,3-dihydro-1H-inden-4-yl)phenyl)-3-(((((R)-5-oxopyrrolidin-2-yl)methyl)amino)methyl)-4H-pyrido[1,2-a]pyrimidin-4-one;
8-(2,2′-dichloro-3′-(6-methoxy-5-(((((S)-5-oxopyrrolidin-2-yl)methyl)amino)methyl)pyridin-2-yl)-[1,1′-biphenyl]-3-yl)-3-(((2-((S)-5-oxopyrrolidin-2-yl)ethyl)amino)methyl)-4H-pyrido[1,2-a]pyrimidin-4-one;
8-(2,2′-dichloro-3′-(6-methoxy-5-(((((R)-5-oxopyrrolidin-2-yl)methyl)amino)methyl)pyridin-2-yl)-[1,1′-biphenyl]-3-yl)-3-(((2-((S)-5-oxopyrrolidin-2-yl)ethyl)amino)methyl)-4H-pyrido[1,2-a]pyrimidin-4-one;
8-(2,2′-dichloro-3′-(6-methoxy-5-(((((S)-5-oxopyrrolidin-2-yl)methyl)amino)methyl)pyridin-2-yl)-[1,1′-biphenyl]-3-yl)-3-(((2-((R)-5-oxopyrrolidin-2-yl)ethyl)amino)methyl)-4H-pyrido[1,2-a]pyrimidin-4-one;
8-(2,2′-dichloro-3′-(6-methoxy-5-(((((R)-5-oxopyrrolidin-2-yl)methyl)amino)methyl)pyridin-2-yl)-[1,1′-biphenyl]-3-yl)-3-(((2-((R)-5-oxopyrrolidin-2-yl)ethyl)amino)methyl)-4H-pyrido[1,2-a]pyrimidin-4-one;
(S)-3-(((2-(1H-imidazol-4-yl)ethyl)amino)methyl)-8-(2,2′-dichloro-3′-(6-methoxy-5-((((5-oxopyrrolidin-2-yl)methyl)amino)methyl)pyridin-2-yl)-[1,1′-biphenyl]-3-yl)-4H-pyrido[1,2-a]pyrimidin-4-one;
(R)-3-(((2-(1H-imidazol-4-yl)ethyl)amino)methyl)-8-(2,2′-dichloro-3′-(6-methoxy-5-((((5-oxopyrrolidin-2-yl)methyl)amino)methyl)pyridin-2-yl)-[1,1′-biphenyl]-3-yl)-4H-pyrido[1,2-a]pyrimidin-4-one;
(S)-3-(((2-(4H-1,2,4-triazol-4-yl)ethyl)amino)methyl)-8-(2,2′-dichloro-3′-(6-methoxy-5-((((5-oxopyrrolidin-2-yl)methyl)amino)methyl)pyridin-2-yl)-[1,1′-biphenyl]-3-yl)-4H-pyrido[1,2-a]pyrimidin-4-one;
(R)-3-(((2-(4H-1,2,4-triazol-4-yl)ethyl)amino)methyl)-8-(2,2′-dichloro-3′-(6-methoxy-5-((((5-oxopyrrolidin-2-yl)methyl)amino)methyl)pyridin-2-yl)-[1,1′-biphenyl]-3-yl)-4H-pyrido[1,2-a]pyrimidin-4-one;
(S)-5-((((6-(3′-(3-amino-2-(hydroxymethyl)thieno[3,2-b]pyridin-6-yl)-2,2′-dichloro-[1,1′-biphenyl]-3-yl)-2-methoxypyridin-3-yl)methyl)amino)methyl)pyrrolidin-2-one;
(R)-5-((((6-(3′-(3-amino-2-(hydroxymethyl)thieno[3,2-b]pyridin-6-yl)-2,2′-dichloro-[1,1′-biphenyl]-3-yl)-2-methoxypyridin-3-yl)methyl)amino)methyl)pyrrolidin-2-one;
(S)-5-((((6-(2,2′-dichloro-3′-(2,3,4,5-tetrahydropyrido[3,2-f][1,4]oxazepin-8-yl)-[1,1′-biphenyl]-3-yl)-2-methoxypyridin-3-yl)methyl)amino)methyl)pyrrolidin-2-one;
(R)-5-((((6-(2,2′-dichloro-3′-(2,3,4,5-tetrahydropyrido[3,2-f][1,4]oxazepin-8-yl)-[1,1′-biphenyl]-3-yl)-2-methoxypyridin-3-yl)methyl)amino)methyl)pyrrolidin-2-one;
(S)-8-(2,2′-dichloro-3′-(6-methoxy-5-((((5-oxopyrrolidin-2-yl)methyl)amino)methyl)pyridin-2-yl)-[1,1′-biphenyl]-3-yl)-3-(((2-(2-oxopyrrolidin-1-yl)ethyl)amino)methyl)-4H-pyrido[1,2-a]pyrimidin-4-one;
(R)-8-(2,2′-dichloro-3′-(6-methoxy-5-((((5-oxopyrrolidin-2-yl)methyl)amino)methyl)pyridin-2-yl)-[1,1′-biphenyl]-3-yl)-3-(((2-(2-oxopyrrolidin-1-yl)ethyl)amino)methyl)-4H-pyrido[1,2-a]pyrimidin-4-one;
methyl (S)-4-(((8-(2,2′-dichloro-3′-(6-methoxy-5-(((((S)-5-oxopyrrolidin-2-yl)methyl)amino)methyl)pyridin-2-yl)-[1,1′-biphenyl]-3-yl)-4-oxo-4H-pyrido[1,2-a]pyrimidin-3-yl)methyl)amino)-3-hydroxybutanoate;
methyl (R)-4-(((8-(2,2′-dichloro-3′-(6-methoxy-5-(((((S)-5-oxopyrrolidin-2-yl)methyl)amino)methyl)pyridin-2-yl)-[1,1′-biphenyl]-3-yl)-4-oxo-4H-pyrido[1,2-a]pyrimidin-3-yl)methyl)amino)-3-hydroxybutanoate;
methyl (S)-4-(((8-(2,2′-dichloro-3′-(6-methoxy-5-(((((R)-5-oxopyrrolidin-2-yl)methyl)amino)methyl)pyridin-2-yl)-[1,1′-biphenyl]-3-yl)-4-oxo-4H-pyrido[1,2-a]pyrimidin-3-yl)methyl)amino)-3-hydroxybutanoate;
methyl (R)-4-(((8-(2,2′-dichloro-3′-(6-methoxy-5-(((((R)-5-oxopyrrolidin-2-yl)methyl)amino)methyl)pyridin-2-yl)-[1,1′-biphenyl]-3-yl)-4-oxo-4H-pyrido[1,2-a]pyrimidin-3-yl)methyl)amino)-3-hydroxybutanoate;
7-(2,2′-dichloro-3′-(6-methoxy-5-(((((S)-5-oxopyrrolidin-2-yl)methyl)amino)methyl)pyridin-2-yl)-[1,1′-biphenyl]-3-yl)-2-((((1r,3r)-3-hydroxycyclobutyl)amino)methyl)-4H-pyrido[1,2-a]pyrimidin-4-one;
7-(2,2′-dichloro-3′-(6-methoxy-5-(((((R)-5-oxopyrrolidin-2-yl)methyl)amino)methyl)pyridin-2-yl)-[1,1′-biphenyl]-3-yl)-2-((((1r,3r)-3-hydroxycyclobutyl)amino)methyl)-4H-pyrido[1,2-a]pyrimidin-4-one;
7-(2,2′-dichloro-3′-(6-methoxy-5-(((((S)-5-oxopyrrolidin-2-yl)methyl)amino)methyl)pyridin-2-yl)-[1,1′-biphenyl]-3-yl)-2-((((1s,3s)-3-hydroxycyclobutyl)amino)methyl)-4H-pyrido[1,2-a]pyrimidin-4-one;
7-(2,2′-dichloro-3′-(6-methoxy-5-(((((R)-5-oxopyrrolidin-2-yl)methyl)amino)methyl)pyridin-2-yl)-[1,1′-biphenyl]-3-yl)-2-((((1s,3s)-3-hydroxycyclobutyl)amino)methyl)-4H-pyrido[1,2-a]pyrimidin-4-one;
(S)-7-(2,2′-dichloro-3′-(6-methoxy-5-((((5-oxopyrrolidin-2-yl)methyl)amino)methyl)pyridin-2-yl)-[1,1′-biphenyl]-3-yl)-2-((3-hydroxy-3-methylazetidin-1-yl)methyl)-4H-pyrido[1,2-a]pyrimidin-4-one;
(R)-7-(2,2′-dichloro-3′-(6-methoxy-5-((((5-oxopyrrolidin-2-yl)methyl)amino)methyl)pyridin-2-yl)-[1,1′-biphenyl]-3-yl)-2-((3-hydroxy-3-methylazetidin-1-yl)methyl)-4H-pyrido[1,2-a]pyrimidin-4-one;
(S)-8-(2,2′-dichloro-3′-(6-methoxy-5-((((5-oxopyrrolidin-2-yl)methyl)amino)methyl)pyridin-2-yl)-[1,1′-biphenyl]-3-yl)-3-(((4,4,5,5,5-pentafluoropentyl)amino)methyl)-4H-pyrido[1,2-a]pyrimidin-4-one;
(R)-8-(2,2′-dichloro-3′-(6-methoxy-5-((((5-oxopyrrolidin-2-yl)methyl)amino)methyl)pyridin-2-yl)-[1,1′-biphenyl]-3-yl)-3-(((4,4,5,5,5-pentafluoropentyl)amino)methyl)-4H-pyrido[1,2-a]pyrimidin-4-one;
(S)-8-(2,2′-dichloro-3′-(6-methoxy-5-((((5-oxopyrrolidin-2-yl)methyl)amino)methyl)pyridin-2-yl)-[1,1′-biphenyl]-3-yl)-3-(((1,1-dioxidotetrahydro-2H-thiopyran-4-yl)amino)methyl)-4H-pyrido[1,2-a]pyrimidin-4-one;
(R)-8-(2,2′-dichloro-3′-(6-methoxy-5-((((5-oxopyrrolidin-2-yl)methyl)amino)methyl)pyridin-2-yl)-[1,1′-biphenyl]-3-yl)-3-(((1,1-dioxidotetrahydro-2H-thiopyran-4-yl)amino)methyl)-4H-pyrido[1,2-a]pyrimidin-4-one;
8-(2,2′-dichloro-3′-(6-methoxy-5-(((((S)-5-oxopyrrolidin-2-yl)methyl)amino)methyl)pyridin-2-yl)-[1,1′-biphenyl]-3-yl)-3-(((((1S,2S)-2-(hydroxymethyl)cyclopropyl)methyl)amino)methyl)-4H-pyrido[1,2-a]pyrimidin-4-one;
8-(2,2′-dichloro-3′-(6-methoxy-5-(((((S)-5-oxopyrrolidin-2-yl)methyl)amino)methyl)pyridin-2-yl)-[1,1′-biphenyl]-3-yl)-3-(((((1R,2S)-2-(hydroxymethyl)cyclopropyl)methyl)amino)methyl)-4H-pyrido[1,2-a]pyrimidin-4-one;
8-(2,2′-dichloro-3′-(6-methoxy-5-(((((S)-5-oxopyrrolidin-2-yl)methyl)amino)methyl)pyridin-2-yl)-[1,1′-biphenyl]-3-yl)-3-(((((1S,2R)-2-(hydroxymethyl)cyclopropyl)methyl)amino)methyl)-4H-pyrido[1,2-a]pyrimidin-4-one;
8-(2,2′-dichloro-3′-(6-methoxy-5-(((((S)-5-oxopyrrolidin-2-yl)methyl)amino)methyl)pyridin-2-yl)-[1,1′-biphenyl]-3-yl)-3-(((((1R,2R)-2-(hydroxymethyl)cyclopropyl)methyl)amino)methyl)-4H-pyrido[1,2-a]pyrimidin-4-one;
8-(2,2′-dichloro-3′-(6-methoxy-5-(((((R)-5-oxopyrrolidin-2-yl)methyl)amino)methyl)pyridin-2-yl)-[1,1′-biphenyl]-3-yl)-3-(((((1S,2S)-2-(hydroxymethyl)cyclopropyl)methyl)amino)methyl)-4H-pyrido[1,2-a]pyrimidin-4-one;
8-(2,2′-dichloro-3′-(6-methoxy-5-(((((R)-5-oxopyrrolidin-2-yl)methyl)amino)methyl)pyridin-2-yl)-[1,1′-biphenyl]-3-yl)-3-(((((1R,2S)-2-(hydroxymethyl)cyclopropyl)methyl)amino)methyl)-4H-pyrido[1,2-a]pyrimidin-4-one;
8-(2,2′-dichloro-3′-(6-methoxy-5-(((((R)-5-oxopyrrolidin-2-yl)methyl)amino)methyl)pyridin-2-yl)-[1,1′-biphenyl]-3-yl)-3-(((((1S,2R)-2-(hydroxymethyl)cyclopropyl)methyl)amino)methyl)-4H-pyrido[1,2-a]pyrimidin-4-one;
8-(2,2′-dichloro-3′-(6-methoxy-5-(((((R)-5-oxopyrrolidin-2-yl)methyl)amino)methyl)pyridin-2-yl)-[1,1′-biphenyl]-3-yl)-3-(((((1R,2R)-2-(hydroxymethyl)cyclopropyl)methyl)amino)methyl)-4H-pyrido[1,2-a]pyrimidin-4-one;
8-(2,2′-dichloro-3′-(6-methoxy-5-(((((S)-5-oxopyrrolidin-2-yl)methyl)amino)methyl)pyridin-2-yl)-[1,1′-biphenyl]-3-yl)-3-((((S)-2-hydroxypropyl)amino)methyl)-6-methyl-4H-pyrido[1,2-a]pyrimidin-4-one;
8-(2,2′-dichloro-3′-(6-methoxy-5-(((((R)-5-oxopyrrolidin-2-yl)methyl)amino)methyl)pyridin-2-yl)-[1,1′-biphenyl]-3-yl)-3-((((S)-2-hydroxypropyl)amino)methyl)-6-methyl-4H-pyrido[1,2-a]pyrimidin-4-one;
8-(2,2′-dichloro-3′-(6-methoxy-5-(((((S)-5-oxopyrrolidin-2-yl)methyl)amino)methyl)pyridin-2-yl)-[1,1′-biphenyl]-3-yl)-3-((((R)-2-hydroxypropyl)amino)methyl)-6-methyl-4H-pyrido[1,2-a]pyrimidin-4-one;
8-(2,2′-dichloro-3′-(6-methoxy-5-(((((R)-5-oxopyrrolidin-2-yl)methyl)amino)methyl)pyridin-2-yl)-[1,1′-biphenyl]-3-yl)-3-((((R)-2-hydroxypropyl)amino)methyl)-6-methyl-4H-pyrido[1,2-a]pyrimidin-4-one;
2-chloro-8-(2,2′-dichloro-3′-(6-methoxy-5-(((((S)-5-oxopyrrolidin-2-yl)methyl)amino)methyl)pyridin-2-yl)-[1,1′-biphenyl]-3-yl)-3-(((((S)-5-oxopyrrolidin-2-yl)methyl)amino)methyl)-4H-pyrido[1,2-a]pyrimidin-4-one;
2-chloro-8-(2,2′-dichloro-3′-(6-methoxy-5-(((((R)-5-oxopyrrolidin-2-yl)methyl)amino)methyl)pyridin-2-yl)-[1,1′-biphenyl]-3-yl)-3-(((((S)-5-oxopyrrolidin-2-yl)methyl)amino)methyl)-4H-pyrido[1,2-a]pyrimidin-4-one;
2-chloro-8-(2,2′-dichloro-3′-(6-methoxy-5-(((((S)-5-oxopyrrolidin-2-yl)methyl)amino)methyl)pyridin-2-yl)-[1,1′-biphenyl]-3-yl)-3-(((((R)-5-oxopyrrolidin-2-yl)methyl)amino)methyl)-4H-pyrido[1,2-a]pyrimidin-4-one;
2-chloro-8-(2,2′-dichloro-3′-(6-methoxy-5-(((((R)-5-oxopyrrolidin-2-yl)methyl)amino)methyl)pyridin-2-yl)-[1,1′-biphenyl]-3-yl)-3-(((((R)-5-oxopyrrolidin-2-yl)methyl)amino)methyl)-4H-pyrido[1,2-a]pyrimidin-4-one;
(S)-5-((((6-(2,2′-dichloro-3′-(((E)-6-methoxy-5-(((((S)-5-oxopyrrolidin-2-yl)methyl)amino)methyl)-2,3-dihydro-1H-inden-1-ylidene)methyl)-[1,1′-biphenyl]-3-yl)-2-methoxypyridin-3-yl)methyl)amino)methyl)pyrrolidin-2-one;
(R)-5-((((6-(2,2′-dichloro-3′-(((E)-6-methoxy-5-(((((S)-5-oxopyrrolidin-2-yl)methyl)amino)methyl)-2,3-dihydro-1H-inden-1-ylidene)methyl)-[1,1′-biphenyl]-3-yl)-2-methoxypyridin-3-yl)methyl)amino)methyl)pyrrolidin-2-one;
(S)-5-((((6-(2,2′-dichloro-3′-(((E)-6-methoxy-5-(((((R)-5-oxopyrrolidin-2-yl)methyl)amino)methyl)-2,3-dihydro-1H-inden-1-ylidene)methyl)-[1,1′-biphenyl]-3-yl)-2-methoxypyridin-3-yl)methyl)amino)methyl)pyrrolidin-2-one;
(R)-5-((((6-(2,2′-dichloro-3′-(((E)-6-methoxy-5-(((((R)-5-oxopyrrolidin-2-yl)methyl)amino)methyl)-2,3-dihydro-1H-inden-1-ylidene)methyl)-[1,1′-biphenyl]-3-yl)-2-methoxypyridin-3-yl)methyl)amino)methyl)pyrrolidin-2-one;
(S)-5-((((6-(2,2′-dichloro-3′-(((Z)-6-methoxy-5-(((((S)-5-oxopyrrolidin-2-yl)methyl)amino)methyl)-2,3-dihydro-1H-inden-1-ylidene)methyl)-[1,1′-biphenyl]-3-yl)-2-methoxypyridin-3-yl)methyl)amino)methyl)pyrrolidin-2-one;
(R)-5-((((6-(2,2′-dichloro-3′-(((Z)-6-methoxy-5-(((((S)-5-oxopyrrolidin-2-yl)methyl)amino)methyl)-2,3-dihydro-1H-inden-1-ylidene)methyl)-[1,1′-biphenyl]-3-yl)-2-methoxypyridin-3-yl)methyl)amino)methyl)pyrrolidin-2-one;
(S)-5-((((6-(2,2′-dichloro-3′-(((Z)-6-methoxy-5-(((((R)-5-oxopyrrolidin-2-yl)methyl)amino)methyl)-2,3-dihydro-1H-inden-1-ylidene)methyl)-[1,1′-biphenyl]-3-yl)-2-methoxypyridin-3-yl)methyl)amino)methyl)pyrrolidin-2-one;
(R)-5-((((6-(2,2′-dichloro-3′-(((Z)-6-methoxy-5-(((((R)-5-oxopyrrolidin-2-yl)methyl)amino)methyl)-2,3-dihydro-1H-inden-1-ylidene)methyl)-[1,1′-biphenyl]-3-yl)-2-methoxypyridin-3-yl)methyl)amino)methyl)pyrrolidin-2-one;
8-(2,2′-dichloro-3′-(6-methoxy-5-(((((S)-5-oxopyrrolidin-2-yl)methyl)amino)methyl)pyridin-2-yl)-[1,1′-biphenyl]-3-yl)-3-((((1s,3s)-3-hydroxy-3-methylcyclobutyl)amino)methyl)-2-methyl-4H-pyrido[1,2-a]pyrimidin-4-one;
8-(2,2′-dichloro-3′-(6-methoxy-5-(((((S)-5-oxopyrrolidin-2-yl)methyl)amino)methyl)pyridin-2-yl)-[1,1′-biphenyl]-3-yl)-3-((((1r,3r)-3-hydroxy-3-methylcyclobutyl)amino)methyl)-2-methyl-4H-pyrido[1,2-a]pyrimidin-4-one;
8-(2,2′-dichloro-3′-(6-methoxy-5-(((((R)-5-oxopyrrolidin-2-yl)methyl)amino)methyl)pyridin-2-yl)-[1,1′-biphenyl]-3-yl)-3-((((1s,3s)-3-hydroxy-3-methylcyclobutyl)amino)methyl)-2-methyl-4H-pyrido[1,2-a]pyrimidin-4-one;
8-(2,2′-dichloro-3′-(6-methoxy-5-(((((R)-5-oxopyrrolidin-2-yl)methyl)amino)methyl)pyridin-2-yl)-[1,1′-biphenyl]-3-yl)-3-((((1r,3r)-3-hydroxy-3-methylcyclobutyl)amino)methyl)-2-methyl-4H-pyrido[1,2-a]pyrimidin-4-one;
8-(2,2′-dichloro-3′-(5-((((1s,3s)-3-hydroxy-3-methylcyclobutyl)amino)methyl)-6-methoxypyridin-2-yl)-[1,1′-biphenyl]-3-yl)-3-((((1s,3s)-3-hydroxy-3-methylcyclobutyl)amino)methyl)-2-methyl-4H-pyrido[1,2-a]pyrimidin-4-one;
8-(2,2′-dichloro-3′-(5-((((1r,3r)-3-hydroxy-3-methylcyclobutyl)amino)methyl)-6-methoxypyridin-2-yl)-[1,1′-biphenyl]-3-yl)-3-((((1s,3s)-3-hydroxy-3-methylcyclobutyl)amino)methyl)-2-methyl-4H-pyrido[1,2-a]pyrimidin-4-one;
8-(2,2′-dichloro-3′-(5-((((1s,3s)-3-hydroxy-3-methylcyclobutyl)amino)methyl)-6-methoxypyridin-2-yl)-[1,1′-biphenyl]-3-yl)-3-((((1r,3r)-3-hydroxy-3-methylcyclobutyl)amino)methyl)-2-methyl-4H-pyrido[1,2-a]pyrimidin-4-one;
8-(2,2′-dichloro-3′-(5-((((1r,3r)-3-hydroxy-3-methylcyclobutyl)amino)methyl)-6-methoxypyridin-2-yl)-[1,1′-biphenyl]-3-yl)-3-((((1r,3r)-3-hydroxy-3-methylcyclobutyl)amino)methyl)-2-methyl-4H-pyrido[1,2-a]pyrimidin-4-one;
methyl (S)-4-(((6-(2,2′-dichloro-3′-(3-((((S)-2-hydroxy-4-methoxy-4-oxobutyl)amino)methyl)-4-oxo-4H-pyrido[1,2-a]pyrimidin-8-yl)-[1,1′-biphenyl]-3-yl)-2-methoxypyridin-3-yl)methyl)amino)-3-hydroxybutanoate;
methyl (R)-4-(((6-(2,2′-dichloro-3′-(3-((((S)-2-hydroxy-4-methoxy-4-oxobutyl)amino)methyl)-4-oxo-4H-pyrido[1,2-a]pyrimidin-8-yl)-[1,1′-biphenyl]-3-yl)-2-methoxypyridin-3-yl)methyl)amino)-3-hydroxybutanoate;
methyl (S)-4-(((6-(2,2′-dichloro-3′-(3-((((R)-2-hydroxy-4-methoxy-4-oxobutyl)amino)methyl)-4-oxo-4H-pyrido[1,2-a]pyrimidin-8-yl)-[1,1′-biphenyl]-3-yl)-2-methoxypyridin-3-yl)methyl)amino)-3-hydroxybutanoate;
methyl (R)-4-(((6-(2,2′-dichloro-3′-(3-((((R)-2-hydroxy-4-methoxy-4-oxobutyl)amino)methyl)-4-oxo-4H-pyrido[1,2-a]pyrimidin-8-yl)-[1,1′-biphenyl]-3-yl)-2-methoxypyridin-3-yl)methyl)amino)-3-hydroxybutanoate;
(S)-5-((((6-(2,2′-dichloro-3′-(4-fluoro-6-methoxy-5-(((((S)-5-oxopyrrolidin-2-yl)methyl)amino)methyl)pyridin-2-yl)-[1,1′-biphenyl]-3-yl)-2-methoxypyridin-3-yl)methyl)amino)methyl)pyrrolidin-2-one;
(R)-5-((((6-(2,2′-dichloro-3′-(4-fluoro-6-methoxy-5-(((((S)-5-oxopyrrolidin-2-yl)methyl)amino)methyl)pyridin-2-yl)-[1,1′-biphenyl]-3-yl)-2-methoxypyridin-3-yl)methyl)amino)methyl)pyrrolidin-2-one;
(S)-5-((((6-(2,2′-dichloro-3′-(4-fluoro-6-methoxy-5-(((((R)-5-oxopyrrolidin-2-yl)methyl)amino)methyl)pyridin-2-yl)-[1,1′-biphenyl]-3-yl)-2-methoxypyridin-3-yl)methyl)amino)methyl)pyrrolidin-2-one;
(R)-5-((((6-(2,2′-dichloro-3′-(4-fluoro-6-methoxy-5-(((((R)-5-oxopyrrolidin-2-yl)methyl)amino)methyl)pyridin-2-yl)-[1,1′-biphenyl]-3-yl)-2-methoxypyridin-3-yl)methyl)amino)methyl)pyrrolidin-2-one;
(S)-8-(2,2′-dichloro-3′-(6-methoxy-5-((((5-oxopyrrolidin-2-yl)methyl)amino)methyl)pyridin-2-yl)-[1,1′-biphenyl]-3-yl)-3-((((1-hydroxycyclobutyl)methyl)amino)methyl)-4H-pyrido[1,2-a]pyrimidin-4-one;
(R)-8-(2,2′-dichloro-3′-(6-methoxy-5-((((5-oxopyrrolidin-2-yl)methyl)amino)methyl)pyridin-2-yl)-[1,1′-biphenyl]-3-yl)-3-((((1-hydroxycyclobutyl)methyl)amino)methyl)-4H-pyrido[1,2-a]pyrimidin-4-one;
(S)-8-(2,2′-dichloro-3′-(6-methoxy-5-((((5-oxopyrrolidin-2-yl)methyl)amino)methyl)pyridin-2-yl)-[1,1′-biphenyl]-3-yl)-3-((((1-hydroxycyclopropyl)methyl)amino)methyl)-4H-pyrido[1,2-a]pyrimidin-4-one;
(R)-8-(2,2′-dichloro-3′-(6-methoxy-5-((((5-oxopyrrolidin-2-yl)methyl)amino)methyl)pyridin-2-yl)-[1,1′-biphenyl]-3-yl)-3-((((1-hydroxycyclopropyl)methyl)amino)methyl)-4H-pyrido[1,2-a]pyrimidin-4-one;
(5S)-5-((((6-(2,2′-dichloro-3′-((R)-5-((((S)-5-oxopyrrolidin-2-yl)methyl)amino)-5,6,7,8-tetrahydroquinolin-2-yl)-[1,1′-biphenyl]-3-yl)-2-methoxypyridin-3-yl)methyl)amino)methyl)pyrrolidin-2-one;
(5R)-5-((((6-(2,2′-dichloro-3′-((R)-5-((((S)-5-oxopyrrolidin-2-yl)methyl)amino)-5,6,7,8-tetrahydroquinolin-2-yl)-[1,1′-biphenyl]-3-yl)-2-methoxypyridin-3-yl)methyl)amino)methyl)pyrrolidin-2-one;
(5S)-5-((((6-(2,2′-dichloro-3′-((R)-5-((((R)-5-oxopyrrolidin-2-yl)methyl)amino)-5,6,7,8-tetrahydroquinolin-2-yl)-[1,1′-biphenyl]-3-yl)-2-methoxypyridin-3-yl)methyl)amino)methyl)pyrrolidin-2-one;
(5R)-5-((((6-(2,2′-dichloro-3′-((R)-5-((((R)-5-oxopyrrolidin-2-yl)methyl)amino)-5,6,7,8-tetrahydroquinolin-2-yl)-[1,1′-biphenyl]-3-yl)-2-methoxypyridin-3-yl)methyl)amino)methyl)pyrrolidin-2-one;
(5S)-5-((((6-(2,2′-dichloro-3′-((S)-5-((((S)-5-oxopyrrolidin-2-yl)methyl)amino)-5,6,7,8-tetrahydroquinolin-2-yl)-[1,1′-biphenyl]-3-yl)-2-methoxypyridin-3-yl)methyl)amino)methyl)pyrrolidin-2-one;
(5R)-5-((((6-(2,2′-dichloro-3′-((S)-5-((((S)-5-oxopyrrolidin-2-yl)methyl)amino)-5,6,7,8-tetrahydroquinolin-2-yl)-[1,1′-biphenyl]-3-yl)-2-methoxypyridin-3-yl)methyl)amino)methyl)pyrrolidin-2-one;
(5S)-5-((((6-(2,2′-dichloro-3′-((S)-5-((((R)-5-oxopyrrolidin-2-yl)methyl)amino)-5,6,7,8-tetrahydroquinolin-2-yl)-[1,1′-biphenyl]-3-yl)-2-methoxypyridin-3-yl)methyl)amino)methyl)pyrrolidin-2-one;
(5R)-5-((((6-(2,2′-dichloro-3′-((S)-5-((((R)-5-oxopyrrolidin-2-yl)methyl)amino)-5,6,7,8-tetrahydroquinolin-2-yl)-[1,1′-biphenyl]-3-yl)-2-methoxypyridin-3-yl)methyl)amino)methyl)pyrrolidin-2-one;
(5S,5'S)-5,5′-(((((2,2′-dichloro-[1,1′-biphenyl]-3,3′-diyl)bis(4-fluoro-2-methoxypyridine-6,3-diyl))bis(methylene))bis(azanediyl))bis(methylene))bis(pyrrolidin-2-one);
(5R,5'S)-5,5′-(((((2,2′-dichloro-[1,1′-biphenyl]-3,3′-diyl)bis(4-fluoro-2-methoxypyridine-6,3-diyl))bis(methylene))bis(azanediyl))bis(methylene))bis(pyrrolidin-2-one);
(5S,5′R)-5,5′-(((((2,2′-dichloro-[1,1′-biphenyl]-3,3′-diyl)bis(4-fluoro-2-methoxypyridine-6,3-diyl))bis(methylene))bis(azanediyl))bis(methylene))bis(pyrrolidin-2-one);
(5R,5′R)-5,5′-(((((2,2′-dichloro-[1,1′-biphenyl]-3,3′-diyl)bis(4-fluoro-2-methoxypyridine-6,3-diyl))bis(methylene))bis(azanediyl))bis(methylene))bis(pyrrolidin-2-one);
(S)-8-(4′-((1-aminocyclopropyl)methyl)-2,2′-dichloro-[1,1′:3′,1″-terphenyl]-3-yl)-3-((((5-oxopyrrolidin-2-yl)methyl)amino)methyl)-4H-pyrido[1,2-a]pyrimidin-4-one;
(R)-8-(4′-((1-aminocyclopropyl)methyl)-2,2′-dichloro-[1,1′:3′,1″-terphenyl]-3-yl)-3-((((5-oxopyrrolidin-2-yl)methyl)amino)methyl)-4H-pyrido[1,2-a]pyrimidin-4-one;
(S)-4-(((8-(2,2′-dichloro-3′-(6-methoxy-5-(((((S)-5-oxopyrrolidin-2-yl)methyl)amino)methyl)pyridin-2-yl)-[1,1′-biphenyl]-3-yl)-4-oxo-4H-pyrido[1,2-a]pyrimidin-3-yl)methyl)amino)-3-hydroxybutanamide;
(R)-4-(((8-(2,2′-dichloro-3′-(6-methoxy-5-(((((S)-5-oxopyrrolidin-2-yl)methyl)amino)methyl)pyridin-2-yl)-[1,1′-biphenyl]-3-yl)-4-oxo-4H-pyrido[1,2-a]pyrimidin-3-yl)methyl)amino)-3-hydroxybutanamide;
(S)-4-(((8-(2,2′-dichloro-3′-(6-methoxy-5-(((((R)-5-oxopyrrolidin-2-yl)methyl)amino)methyl)pyridin-2-yl)-[1,1′-biphenyl]-3-yl)-4-oxo-4H-pyrido[1,2-a]pyrimidin-3-yl)methyl)amino)-3-hydroxybutanamide;
(R)-4-(((8-(2,2′-dichloro-3′-(6-methoxy-5-(((((R)-5-oxopyrrolidin-2-yl)methyl)amino)methyl)pyridin-2-yl)-[1,1′-biphenyl]-3-yl)-4-oxo-4H-pyrido[1,2-a]pyrimidin-3-yl)methyl)amino)-3-hydroxybutanamide;
(S)-6-(2,2′-dichloro-3′-(6-methoxy-5-((((5-oxopyrrolidin-2-yl)methyl)amino)methyl)pyridin-2-yl)-[1,1′-biphenyl]-3-yl)-2-(2-hydroxyethyl)-[1,2,4]triazolo[4,3-a]pyridin-3(2H)-one;
(R)-6-(2,2′-dichloro-3′-(6-methoxy-5-((((5-oxopyrrolidin-2-yl)methyl)amino)methyl)pyridin-2-yl)-[1,1′-biphenyl]-3-yl)-2-(2-hydroxyethyl)-[1,2,4]triazolo[4,3-a]pyridin-3(2H)-one;
(S)-5-((((6-(2,2′-dichloro-3′-(5,6,7,8-tetrahydro-[1,2,4]triazolo[4,3-a]pyrazin-3-yl)-[1,1′-biphenyl]-3-yl)-2-methoxypyridin-3-yl)methyl)amino)methyl)pyrrolidin-2-one;
(R)-5-((((6-(2,2′-dichloro-3′-(5,6,7,8-tetrahydro-[1,2,4]triazolo[4,3-a]pyrazin-3-yl)-[1,1′-biphenyl]-3-yl)-2-methoxypyridin-3-yl)methyl)amino)methyl)pyrrolidin-2-one;
(S)-5-((((6-(2,2′-dichloro-3′-(2-(4-(2-hydroxyethyl)piperazin-1-yl)imidazo[2,1-b][1,3,4]thiadiazol-5-yl)-[1,1′-biphenyl]-3-yl)-2-methoxypyridin-3-yl)methyl)amino)methyl)pyrrolidin-2-one;
(R)-5-((((6-(2,2′-dichloro-3′-(2-(4-(2-hydroxyethyl)piperazin-1-yl)imidazo[2,1-b][1,3,4]thiadiazol-5-yl)-[1,1′-biphenyl]-3-yl)-2-methoxypyridin-3-yl)methyl)amino)methyl)pyrrolidin-2-one;
(S)-5-((((6-(2,2′-dichloro-3′-(7-(2-hydroxyethyl)-5,6,7,8-tetrahydro-[1,2,4]triazolo[4,3-a]pyrazin-3-yl)-[1,1′-biphenyl]-3-yl)-2-methoxypyridin-3-yl)methyl)amino)methyl)pyrrolidin-2-one;
(R)-5-((((6-(2,2′-dichloro-3′-(7-(2-hydroxyethyl)-5,6,7,8-tetrahydro-[1,2,4]triazolo[4,3-a]pyrazin-3-yl)-[1,1′-biphenyl]-3-yl)-2-methoxypyridin-3-yl)methyl)amino)methyl)pyrrolidin-2-one;
(S)-8-(2,2′-dichloro-3′-(6-methoxy-5-((((5-oxopyrrolidin-2-yl)methyl)amino)methyl)pyridin-2-yl)-[1,1′-biphenyl]-3-yl)-3-((((3-hydroxyoxetan-3-yl)methyl)amino)methyl)-4H-pyrido[1,2-a]pyrimidin-4-one;
(R)-8-(2,2′-dichloro-3′-(6-methoxy-5-((((5-oxopyrrolidin-2-yl)methyl)amino)methyl)pyridin-2-yl)-[1,1′-biphenyl]-3-yl)-3-((((3-hydroxyoxetan-3-yl)methyl)amino)methyl)-4H-pyrido[1,2-a]pyrimidin-4-one;
(S)-8-(2,2′-dichloro-3′-(6-methoxy-5-((((5-oxopyrrolidin-2-yl)methyl)amino)methyl)pyridin-2-yl)-[1,1′-biphenyl]-3-yl)-3-((((3-(hydroxymethyl)oxetan-3-yl)methyl)amino)methyl)-4H-pyrido[1,2-a]pyrimidin-4-one;
(R)-8-(2,2′-dichloro-3′-(6-methoxy-5-((((5-oxopyrrolidin-2-yl)methyl)amino)methyl)pyridin-2-yl)-[1,1′-biphenyl]-3-yl)-3-((((3-(hydroxymethyl)oxetan-3-yl)methyl)amino)methyl)-4H-pyrido[1,2-a]pyrimidin-4-one;
(S)-8-(2,2′-dichloro-3′-(6-methoxy-5-((((5-oxopyrrolidin-2-yl)methyl)amino)methyl)pyridin-2-yl)-[1,1′-biphenyl]-3-yl)-3-((((3-methyloxetan-3-yl)methyl)amino)methyl)-4H-pyrido[1,2-a]pyrimidin-4-one;
(R)-8-(2,2′-dichloro-3′-(6-methoxy-5-((((5-oxopyrrolidin-2-yl)methyl)amino)methyl)pyridin-2-yl)-[1,1′-biphenyl]-3-yl)-3-((((3-methyloxetan-3-yl)methyl)amino)methyl)-4H-pyrido[1,2-a]pyrimidin-4-one;
(S)-5-((((6-(2,2′-dichloro-3′-(2-(((((S)-5-oxopyrrolidin-2-yl)methyl)amino)methyl)benzo[d]oxazol-5-yl)-[1,1′-biphenyl]-3-yl)-2-methoxypyridin-3-yl)methyl)amino)methyl)pyrrolidin-2-one;
(R)-5-((((6-(2,2′-dichloro-3′-(2-(((((S)-5-oxopyrrolidin-2-15 yl)methyl)amino)methyl)benzo[d]oxazol-5-yl)-[1,1′-biphenyl]-3-yl)-2-methoxypyridin-3-yl)methyl)amino)methyl)pyrrolidin-2-one;
(S)-5-((((6-(2,2′-dichloro-3′-(2-(((((R)-5-oxopyrrolidin-2-yl)methyl)amino)methyl)benzo[d]oxazol-5-yl)-[1,1′-biphenyl]-3-yl)-2-methoxypyridin-3-yl)methyl)amino)methyl)pyrrolidin-2-one;
(R)-5-((((6-(2,2′-dichloro-3′-(2-(((((R)-5-oxopyrrolidin-2-yl)methyl)amino)methyl)benzo[d]oxazol-5-yl)-[1,1′-biphenyl]-3-yl)-2-methoxypyridin-3-yl)methyl)amino)methyl)pyrrolidin-2-one;
(S)-5-((((6-(2,2′-dichloro-3′-(2-(((((S)-5-oxopyrrolidin-2-yl)methyl)amino)methyl)benzo[d]oxazol-6-yl)-[1,1′-biphenyl]-3-yl)-2-methoxypyridin-3-yl)methyl)amino)methyl)pyrrolidin-2-one;
(R)-5-((((6-(2,2′-dichloro-3′-(2-(((((S)-5-oxopyrrolidin-2-yl)methyl)amino)methyl)benzo[d]oxazol-6-yl)-[1,1′-biphenyl]-3-yl)-2-methoxypyridin-3-yl)methyl)amino)methyl)pyrrolidin-2-one;
(S)-5-((((6-(2,2′-dichloro-3′-(2-(((((R)-5-oxopyrrolidin-2-30 yl)methyl)amino)methyl)benzo[d]oxazol-6-yl)-[1,1′-biphenyl]-3-yl)-2-methoxypyridin-3-yl)methyl)amino)methyl)pyrrolidin-2-one;
(R)-5-((((6-(2,2′-dichloro-3′-(2-(((((R)-5-oxopyrrolidin-2-yl)methyl)amino)methyl)benzo[d]oxazol-6-yl)-[1,1′-biphenyl]-3-yl)-2-methoxypyridin-3-yl)methyl)amino)methyl)pyrrolidin-2-one;
(S)-8-(2,2′-dichloro-3′-(6-methoxy-5-(((((S)-5-oxopyrrolidin-2-yl)methyl)amino)methyl)pyridin-2-yl)-[1,1′-biphenyl]-3-yl)-3-(((5-oxo-6-azaspiro[3.4]octan-2-yl)amino)methyl)-4H-pyrido[1,2-a]pyrimidin-4-one;
(R)-8-(2,2′-dichloro-3′-(6-methoxy-5-(((((S)-5-oxopyrrolidin-2-yl)methyl)amino)methyl)pyridin-2-yl)-[1,1′-biphenyl]-3-yl)-3-(((5-oxo-6-azaspiro[3.4]octan-2-yl)amino)methyl)-4H-pyrido[1,2-a]pyrimidin-4-one;
(S)-8-(2,2′-dichloro-3′-(6-methoxy-5-(((((R)-5-oxopyrrolidin-2-yl)methyl)amino)methyl)pyridin-2-yl)-[1,1′-biphenyl]-3-yl)-3-(((5-oxo-6-azaspiro[3.4]octan-2-yl)amino)methyl)-4H-pyrido[1,2-a]pyrimidin-4-one;
(R)-8-(2,2′-dichloro-3′-(6-methoxy-5-(((((R)-5-oxopyrrolidin-2-yl)methyl)amino)methyl)pyridin-2-yl)-[1,1′-biphenyl]-3-yl)-3-(((5-oxo-6-azaspiro[3.4]octan-2-yl)amino)methyl)-4H-pyrido[1,2-a]pyrimidin-4-one;
8-(2,2′-dichloro-3′-(6-methoxy-5-(((((S)-5-oxopyrrolidin-2-yl)methyl)amino)methyl)pyridin-2-yl)-[1,1′-biphenyl]-3-yl)-3-((((3-oxo-2-azabicyclo[2.2.1]heptan-1-yl)methyl)amino)methyl)-4H-pyrido[1,2-a]pyrimidin-4-one;
8-(2,2′-dichloro-3′-(6-methoxy-5-(((((R)-5-oxopyrrolidin-2-yl)methyl)amino)methyl)pyridin-2-yl)-[1,1′-biphenyl]-3-yl)-3-((((3-oxo-2-azabicyclo[2.2.1]heptan-1-yl)methyl)amino)methyl)-4H-pyrido[1,2-a]pyrimidin-4-one;
8-(2,2′-dichloro-3′-(6-methoxy-5-(((((S)-5-oxopyrrolidin-2-yl)methyl)amino)methyl)pyridin-2-yl)-[1,1′-biphenyl]-3-yl)-3-(((((S)-4,4-difluoro-5-oxopyrrolidin-2-yl)methyl)amino)methyl)-4H-pyrido[1,2-a]pyrimidin-4-one;
8-(2,2′-dichloro-3′-(6-methoxy-5-(((((S)-5-oxopyrrolidin-2-yl)methyl)amino)methyl)pyridin-2-yl)-[1,1′-biphenyl]-3-yl)-3-(((((R)-4,4-difluoro-5-oxopyrrolidin-2-yl)methyl)amino)methyl)-4H-pyrido[1,2-a]pyrimidin-4-one;
8-(2,2′-dichloro-3′-(6-methoxy-5-(((((R)-5-oxopyrrolidin-2-yl)methyl)amino)methyl)pyridin-2-yl)-[1,1′-biphenyl]-3-yl)-3-(((((S)-4,4-difluoro-5-oxopyrrolidin-2-yl)methyl)amino)methyl)-4H-pyrido[1,2-a]pyrimidin-4-one;
8-(2,2′-dichloro-3′-(6-methoxy-5-(((((R)-5-oxopyrrolidin-2-yl)methyl)amino)methyl)pyridin-2-yl)-[1,1′-biphenyl]-3-yl)-3-(((((R)-4,4-difluoro-5-oxopyrrolidin-2-yl)methyl)amino)methyl)-4H-pyrido[1,2-a]pyrimidin-4-one;
8-(2,2′-dichloro-3′-(6-methoxy-5-(((((S)-5-oxopyrrolidin-2-yl)methyl)amino)methyl)pyridin-2-yl)-[1,1′-biphenyl]-3-yl)-3-((((1s,3s)-3-hydroxy-3-methylcyclobutyl)amino)methyl)-6-methyl-4H-pyrido[1,2-a]pyrimidin-4-one;
8-(2,2′-dichloro-3′-(6-methoxy-5-(((((R)-5-oxopyrrolidin-2-yl)methyl)amino)methyl)pyridin-2-yl)-[1,1′-biphenyl]-3-yl)-3-((((1s,3s)-3-hydroxy-3-methylcyclobutyl)amino)methyl)-6-methyl-4H-pyrido[1,2-a]pyrimidin-4-one;
8-(2,2′-dichloro-3′-(6-methoxy-5-(((((S)-5-oxopyrrolidin-2-yl)methyl)amino)methyl)pyridin-2-yl)-[1,1′-biphenyl]-3-yl)-3-((((1r,3r)-3-hydroxy-3-methylcyclobutyl)amino)methyl)-6-methyl-4H-pyrido[1,2-a]pyrimidin-4-one;
8-(2,2′-dichloro-3′-(6-methoxy-5-(((((R)-5-oxopyrrolidin-2-yl)methyl)amino)methyl)pyridin-2-yl)-[1,1′-biphenyl]-3-yl)-3-((((1r,3r)-3-hydroxy-3-methylcyclobutyl)amino)methyl)-6-methyl-4H-pyrido[1,2-a]pyrimidin-4-one;
(S)-1-((8-(2,2′-dichloro-3′-(6-methoxy-5-((((5-oxopyrrolidin-2-yl)methyl)amino)methyl)pyridin-2-yl)-[1,1′-biphenyl]-3-yl)-4-oxo-4H-pyrido[1,2-a]pyrimidin-3-yl)methyl)-N-methylazetidine-3-carboxamide;
(R)-1-((8-(2,2′-dichloro-3′-(6-methoxy-5-((((5-oxopyrrolidin-2-yl)methyl)amino)methyl)pyridin-2-yl)-[1,1′-biphenyl]-3-yl)-4-oxo-4H-pyrido[1,2-a]pyrimidin-3-yl)methyl)-N-methylazetidine-3-carboxamide;
(S)-3-(((1-acetylpiperidin-4-yl)amino)methyl)-8-(2,2′-dichloro-3′-(6-methoxy-5-((((5-oxopyrrolidin-2-yl)methyl)amino)methyl)pyridin-2-yl)-[1,1′-biphenyl]-3-yl)-4H-pyrido[1,2-a]pyrimidin-4-one;
(R)-3-(((1-acetylpiperidin-4-yl)amino)methyl)-8-(2,2′-dichloro-3′-(6-methoxy-5-((((5-oxopyrrolidin-2-yl)methyl)amino)methyl)pyridin-2-yl)-[1,1′-biphenyl]-3-yl)-4H-pyrido[1,2-a]pyrimidin-4-one;
(S)-1-((8-(2,2′-dichloro-3′-(6-methoxy-5-(((((S)-5-oxopyrrolidin-2-yl)methyl)amino)methyl)pyridin-2-yl)-[1,1′-biphenyl]-3-yl)-4-oxo-4H-pyrido[1,2-a]pyrimidin-3-yl)methyl)-N-methylpyrrolidine-3-carboxamide;
(R)-1-((8-(2,2′-dichloro-3′-(6-methoxy-5-(((((S)-5-oxopyrrolidin-2-yl)methyl)amino)methyl)pyridin-2-yl)-[1,1′-biphenyl]-3-yl)-4-oxo-4H-pyrido[1,2-a]pyrimidin-3-yl)methyl)-N-methylpyrrolidine-3-carboxamide;
(S)-1-((8-(2,2′-dichloro-3′-(6-methoxy-5-(((((R)-5-oxopyrrolidin-2-yl)methyl)amino)methyl)pyridin-2-yl)-[1,1′-biphenyl]-3-yl)-4-oxo-4H-pyrido[1,2-a]pyrimidin-3-yl)methyl)-N-methylpyrrolidine-3-carboxamide;
(R)-1-((8-(2,2′-dichloro-3′-(6-methoxy-5-(((((R)-5-oxopyrrolidin-2-yl)methyl)amino)methyl)pyridin-2-yl)-[1,1′-biphenyl]-3-yl)-4-oxo-4H-pyrido[1,2-a]pyrimidin-3-yl)methyl)-N-methylpyrrolidine-3-carboxamide;
(S)-5-((((6-(2,2′-dichloro-3′-(3-(((((S)-5-oxopyrrolidin-2-yl)methyl)amino)methyl)-1,6-naphthyridin-7-yl)-[1,1′-biphenyl]-3-yl)-2-methoxypyridin-3-yl)methyl)amino)methyl)pyrrolidin-2-one;
(R)-5-((((6-(2,2′-dichloro-3′-(3-(((((S)-5-oxopyrrolidin-2-yl)methyl)amino)methyl)-1,6-naphthyridin-7-yl)-[1,1′-biphenyl]-3-yl)-2-methoxypyridin-3-yl)methyl)amino)methyl)pyrrolidin-2-one;
(S)-5-((((6-(2,2′-dichloro-3′-(3-(((((R)-5-oxopyrrolidin-2-yl)methyl)amino)methyl)-1,6-naphthyridin-7-yl)-[1,1′-biphenyl]-3-yl)-2-methoxypyridin-3-yl)methyl)amino)methyl)pyrrolidin-2-one;
(R)-5-((((6-(2,2′-dichloro-3′-(3-(((((R)-5-oxopyrrolidin-2-yl)methyl)amino)methyl)-1,6-naphthyridin-7-yl)-[1,1′-biphenyl]-3-yl)-2-methoxypyridin-3-yl)methyl)amino)methyl)pyrrolidin-2-one;
1-((8-(2,2′-dichloro-3′-(6-methoxy-5-(((((S)-5-oxopyrrolidin-2-yl)methyl)amino)methyl)pyridin-2-yl)-[1,1′-biphenyl]-3-yl)-4-oxo-4H-pyrido[1,2-a]pyrimidin-3-yl)methyl)azetidine-(2S)-carboxamide;
1-((8-(2,2′-dichloro-3′-(6-methoxy-5-(((((R)-5-oxopyrrolidin-2-yl)methyl)amino)methyl)pyridin-2-yl)-[1,1′-biphenyl]-3-yl)-4-oxo-4H-pyrido[1,2-a]pyrimidin-3-yl)methyl)azetidine-(2S)-carboxamide;
1-((8-(2,2′-dichloro-3′-(6-methoxy-5-(((((S)-5-oxopyrrolidin-2-yl)methyl)amino)methyl)pyridin-2-yl)-[1,1′-biphenyl]-3-yl)-4-oxo-4H-pyrido[1,2-a]pyrimidin-3-yl)methyl)azetidine-(2R)-carboxamide;
1-((8-(2,2′-dichloro-3′-(6-methoxy-5-(((((R)-5-oxopyrrolidin-2-yl)methyl)amino)methyl)pyridin-2-yl)-[1,1′-biphenyl]-3-yl)-4-oxo-4H-pyrido[1,2-a]pyrimidin-3-yl)methyl)azetidine-(2R)-carboxamide;
(S)-2-(((8-(2,2′-dichloro-3′-(6-methoxy-5-(((((S)-5-oxopyrrolidin-2-yl)methyl)amino)methyl)pyridin-2-yl)-[1,1′-biphenyl]-3-yl)-4-oxo-4H-pyrido[1,2-a]pyrimidin-3-yl)methyl)amino)propenamide;
(R)-2-(((8-(2,2′-dichloro-3′-(6-methoxy-5-(((((S)-5-oxopyrrolidin-2-yl)methyl)amino)methyl)pyridin-2-yl)-[1,1′-biphenyl]-3-yl)-4-oxo-4H-pyrido[1,2-a]pyrimidin-3-yl)methyl)amino)propenamide;
(S)-2-(((8-(2,2′-dichloro-3′-(6-methoxy-5-(((((R)-5-oxopyrrolidin-2-yl)methyl)amino)methyl)pyridin-2-yl)-[1,1′-biphenyl]-3-yl)-4-oxo-4H-pyrido[1,2-a]pyrimidin-3-yl)methyl)amino)propenamide;
(R)-2-(((8-(2,2′-dichloro-3′-(6-methoxy-5-(((((R)-5-oxopyrrolidin-2-yl)methyl)amino)methyl)pyridin-2-yl)-[1,1′-biphenyl]-3-yl)-4-oxo-4H-pyrido[1,2-a]pyrimidin-3-yl)methyl)amino)propenamide;
(S)-8-(2,2′-dichloro-3′-(2,3,4,5-tetrahydropyrido[3,2-f][1,4]oxazepin-8-yl)-[1,1′-biphenyl]-3-yl)-3-((((5-oxopyrrolidin-2-yl)methyl)amino)methyl)-4H-pyrido[1,2-a]pyrimidin-4-one;
(R)-8-(2,2′-dichloro-3′-(2,3,4,5-tetrahydropyrido[3,2-f][1,4]oxazepin-8-yl)-[1,1′-biphenyl]-3-yl)-3-((((5-oxopyrrolidin-2-yl)methyl)amino)methyl)-4H-pyrido[1,2-a]pyrimidin-4-one;
8-(2,2′-dichloro-3′-(6-methoxy-5-(((((S)-5-oxopyrrolidin-2-yl)methyl)amino)methyl)pyridin-2-yl)-[1,1′-biphenyl]-3-yl)-3-((((S)-2-hydroxypropyl)amino)methyl)-2-methoxy-4H-pyrido[1,2-a]pyrimidin-4-one;
8-(2,2′-dichloro-3′-(6-methoxy-5-(((((R)-5-oxopyrrolidin-2-yl)methyl)amino)methyl)pyridin-2-yl)-[1,1′-biphenyl]-3-yl)-3-((((S)-2-hydroxypropyl)amino)methyl)-2-methoxy-4H-pyrido[1,2-a]pyrimidin-4-one;
8-(2,2′-dichloro-3′-(6-methoxy-5-(((((S)-5-oxopyrrolidin-2-yl)methyl)amino)methyl)pyridin-2-yl)-[1,1′-biphenyl]-3-yl)-3-((((R)-2-hydroxypropyl)amino)methyl)-2-methoxy-4H-pyrido[1,2-a]pyrimidin-4-one;
8-(2,2′-dichloro-3′-(6-methoxy-5-(((((R)-5-oxopyrrolidin-2-yl)methyl)amino)methyl)pyridin-2-yl)-[1,1′-biphenyl]-3-yl)-3-((((R)-2-hydroxypropyl)amino)methyl)-2-methoxy-4H-pyrido[1,2-a]pyrimidin-4-one;
(S)-2-(((8-(2,2′-dichloro-3′-(6-methoxy-5-(((((S)-5-oxopyrrolidin-2-yl)methyl)amino)methyl)pyridin-2-yl)-[1,1′-biphenyl]-3-yl)-4-oxo-4H-pyrido[1,2-a]pyrimidin-3-yl)methyl)(methyl)amino)-N-methylpropanamide;
(R)-2-(((8-(2,2′-dichloro-3′-(6-methoxy-5-(((((S)-5-oxopyrrolidin-2-yl)methyl)amino)methyl)pyridin-2-yl)-[1,1′-biphenyl]-3-yl)-4-oxo-4H-pyrido[1,2-a]pyrimidin-3-yl)methyl)(methyl)amino)-N-methylpropanamide;
(S)-2-(((8-(2,2′-dichloro-3′-(6-methoxy-5-(((((R)-5-oxopyrrolidin-2-yl)methyl)amino)methyl)pyridin-2-yl)-[1,1′-biphenyl]-3-yl)-4-oxo-4H-pyrido[1,2-a]pyrimidin-3-yl)methyl)(methyl)amino)-N-methylpropanamide;
(R)-2-(((8-(2,2′-dichloro-3′-(6-methoxy-5-(((((R)-5-oxopyrrolidin-2-yl)methyl)amino)methyl)pyridin-2-yl)-[1,1′-biphenyl]-3-yl)-4-oxo-4H-pyrido[1,2-a]pyrimidin-3-yl)methyl)(methyl)amino)-N-methylpropanamide;
(S)-8-(2,2′-dichloro-3′-(6-methoxy-5-((((5-oxopyrrolidin-2-yl)methyl)amino)methyl)pyridin-2-yl)-[1,1′-biphenyl]-3-yl)-3-(((2-(3-oxopiperazin-1-yl)ethyl)amino)methyl)-4H-pyrido[1,2-a]pyrimidin-4-one;
(R)-8-(2,2′-dichloro-3′-(6-methoxy-5-((((5-oxopyrrolidin-2-yl)methyl)amino)methyl)pyridin-2-yl)-[1,1′-biphenyl]-3-yl)-3-(((2-(3-oxopiperazin-1-yl)ethyl)amino)methyl)-4H-pyrido[1,2-a]pyrimidin-4-one;
methyl (S)-1-((8-(2,2′-dichloro-3′-(6-methoxy-5-(((((S)-5-oxopyrrolidin-2-yl)methyl)amino)methyl)pyridin-2-yl)-[1,1′-biphenyl]-3-yl)-2-methyl-4-oxo-4H-pyrido[1,2-a]pyrimidin-3-yl)methyl)pyrrolidine-3-carboxylate;
methyl (R)-1-((8-(2,2′-dichloro-3′-(6-methoxy-5-(((((S)-5-oxopyrrolidin-2-yl)methyl)amino)methyl)pyridin-2-yl)-[1,1′-biphenyl]-3-yl)-2-methyl-4-oxo-4H-pyrido[1,2-a]pyrimidin-3-yl)methyl)pyrrolidine-3-carboxylate;
methyl (S)-1-((8-(2,2′-dichloro-3′-(6-methoxy-5-(((((R)-5-oxopyrrolidin-2-yl)methyl)amino)methyl)pyridin-2-yl)-[1,1′-biphenyl]-3-yl)-2-methyl-4-oxo-4H-pyrido[1,2-a]pyrimidin-3-yl)methyl)pyrrolidine-3-carboxylate;
methyl (R)-1-((8-(2,2′-dichloro-3′-(6-methoxy-5-(((((R)-5-oxopyrrolidin-2-yl)methyl)amino)methyl)pyridin-2-yl)-[1,1′-biphenyl]-3-yl)-2-methyl-4-oxo-4H-pyrido[1,2-a]pyrimidin-3-yl)methyl)pyrrolidine-3-carboxylate;
(S)-8-(2,2′-dichloro-3′-(6-methoxy-5-((((5-oxopyrrolidin-2-yl)methyl)amino)methyl)pyridin-2-yl)-[1,1′-biphenyl]-3-yl)-3-(((2-(1,1-dioxidothiomorpholino)ethyl)amino)methyl)-4H-pyrido[1,2-a]pyrimidin-4-one;
(R)-8-(2,2′-dichloro-3′-(6-methoxy-5-((((5-oxopyrrolidin-2-yl)methyl)amino)methyl)pyridin-2-yl)-[1,1′-biphenyl]-3-yl)-3-(((2-(1,1-dioxidothiomorpholino)ethyl)amino)methyl)-4H-pyrido[1,2-a]pyrimidin-4-one;
(S)-5-((((6-(2,2′-dichloro-3′-(4-(2-hydroxyethyl)-2,3,4,5-tetrahydropyrido[3,2-f1][1,4]oxazepin-8-yl)-[1,1′-biphenyl]-3-yl)-2-methoxypyridin-3-yl)methyl)amino)methyl)pyrrolidin-2-one;
(R)-5-((((6-(2,2′-dichloro-3′-(4-(2-hydroxyethyl)-2,3,4,5-tetrahydropyrido[3,2-f][1,4]oxazepin-8-yl)-[1,1′-biphenyl]-3-yl)-2-methoxypyridin-3-yl)methyl)amino)methyl)pyrrolidin-2-one;
(S)-8-(2,2′-dichloro-3′-(4-(2-hydroxyethyl)-2,3,4,5-tetrahydropyrido[3,2-f][1,4]oxazepin-8-yl)-[1,1′-biphenyl]-3-yl)-3-((((5-oxopyrrolidin-2-yl)methyl)amino)methyl)-4H-pyrido[1,2-a]pyrimidin-4-one;
(R)-8-(2,2′-dichloro-3′-(4-(2-hydroxyethyl)-2,3,4,5-tetrahydropyrido[3,2-f][1,4]oxazepin-8-yl)-[1,1′-biphenyl]-3-yl)-3-((((5-oxopyrrolidin-2-yl)methyl)amino)methyl)-4H-pyrido[1,2-a]pyrimidin-4-one;
methyl (S)-((8-(2,2′-dichloro-3′-(6-methoxy-5-((((5-oxopyrrolidin-2-yl)methyl)amino)methyl)pyridin-2-yl)-[1,1′-biphenyl]-3-yl)-2-methyl-4-oxo-4H-pyrido[1,2-a]pyrimidin-3-yl)methyl)glycinate;
methyl (R)-((8-(2,2′-dichloro-3′-(6-methoxy-5-((((5-oxopyrrolidin-2-yl)methyl)amino)methyl)pyridin-2-yl)-[1,1′-biphenyl]-3-yl)-2-methyl-4-oxo-4H-pyrido[1,2-a]pyrimidin-3-yl)methyl)glycinate;
(S)-4-(((8-(2,2′-dichloro-3′-(4-fluoro-6-methoxy-5-(((((S)-5-oxopyrrolidin-2-yl)methyl)amino)methyl)pyridin-2-yl)-[1,1′-biphenyl]-3-yl)-4-oxo-4H-pyrido[1,2-a]pyrimidin-3-yl)methyl)amino)-3-hydroxybutanoic acid;
(R)-4-(((8-(2,2′-dichloro-3′-(4-fluoro-6-methoxy-5-(((((S)-5-oxopyrrolidin-2-yl)methyl)amino)methyl)pyridin-2-yl)-[1,1′-biphenyl]-3-yl)-4-oxo-4H-pyrido[1,2-a]pyrimidin-3-yl)methyl)amino)-3-hydroxybutanoic acid;
(S)-4-(((8-(2,2′-dichloro-3′-(4-fluoro-6-methoxy-5-(((((R)-5-oxopyrrolidin-2-yl)methyl)amino)methyl)pyridin-2-yl)-[1,1′-biphenyl]-3-yl)-4-oxo-4H-pyrido[1,2-a]pyrimidin-3-yl)methyl)amino)-3-hydroxybutanoic acid;
(R)-4-(((8-(2,2′-dichloro-3′-(4-fluoro-6-methoxy-5-(((((R)-5-oxopyrrolidin-2-yl)methyl)amino)methyl)pyridin-2-yl)-[1,1′-biphenyl]-3-yl)-4-oxo-4H-pyrido[1,2-a]pyrimidin-3-yl)methyl)amino)-3-hydroxybutanoic acid;
methyl (S)-4-(((8-(2,2′-dichloro-3′-(4-fluoro-6-methoxy-5-(((((S)-5-oxopyrrolidin-2-yl)methyl)amino)methyl)pyridin-2-yl)-[1,1′-biphenyl]-3-yl)-4-oxo-4H-pyrido[1,2-a]pyrimidin-3-yl)methyl)amino)-3-hydroxybutanoate;
methyl (R)-4-(((8-(2,2′-dichloro-3′-(4-fluoro-6-methoxy-5-(((((S)-5-oxopyrrolidin-2-yl)methyl)amino)methyl)pyridin-2-yl)-[1,1′-biphenyl]-3-yl)-4-oxo-4H-pyrido[1,2-a]pyrimidin-3-yl)methyl)amino)-3-hydroxybutanoate;
methyl (S)-4-(((8-(2,2′-dichloro-3′-(4-fluoro-6-methoxy-5-(((((R)-5-oxopyrrolidin-2-yl)methyl)amino)methyl)pyridin-2-yl)-[1,1′-biphenyl]-3-yl)-4-oxo-4H-pyrido[1,2-a]pyrimidin-3-yl)methyl)amino)-3-hydroxybutanoate;
methyl (R)-4-(((8-(2,2′-dichloro-3′-(4-fluoro-6-methoxy-5-(((((R)-5-oxopyrrolidin-2-yl)methyl)amino)methyl)pyridin-2-yl)-[1,1′-biphenyl]-3-yl)-4-oxo-4H-pyrido[1,2-a]pyrimidin-3-yl)methyl)amino)-3-hydroxybutanoate;
8-(2,2′-dichloro-3′-(4-fluoro-6-methoxy-5-(((((S)-5-oxopyrrolidin-2-yl)methyl)amino)methyl)pyridin-2-yl)-[1,1′-biphenyl]-3-yl)-3-((((S)-2-hydroxypropyl)amino)methyl)-4H-pyrido[1,2-a]pyrimidin-4-one;
8-(2,2′-dichloro-3′-(4-fluoro-6-methoxy-5-(((((R)-5-oxopyrrolidin-2-yl)methyl)amino)methyl)pyridin-2-yl)-[1,1′-biphenyl]-3-yl)-3-((((S)-2-hydroxypropyl)amino)methyl)-4H-pyrido[1,2-a]pyrimidin-4-one;
8-(2,2′-dichloro-3′-(4-fluoro-6-methoxy-5-(((((S)-5-oxopyrrolidin-2-yl)methyl)amino)methyl)pyridin-2-yl)-[1,1′-biphenyl]-3-yl)-3-((((R)-2-hydroxypropyl)amino)methyl)-4H-pyrido[1,2-a]pyrimidin-4-one;
8-(2,2′-dichloro-3′-(4-fluoro-6-methoxy-5-(((((R)-5-oxopyrrolidin-2-yl)methyl)amino)methyl)pyridin-2-yl)-[1,1′-biphenyl]-3-yl)-3-((((R)-2-hydroxypropyl)amino)methyl)-4H-pyrido[1,2-a]pyrimidin-4-one;
(S)-8-(2,2′-dichloro-3′-(6-methoxy-5-((((5-oxopyrrolidin-2-yl)methyl)amino)methyl)pyridin-2-yl)-[1,1′-biphenyl]-3-yl)-3-(((3-hydroxy-2,2-dimethylpropyl)amino)methyl)-4H-pyrido[1,2-a]pyrimidin-4-one;
(R)-8-(2,2′-dichloro-3′-(6-methoxy-5-((((5-oxopyrrolidin-2-yl)methyl)amino)methyl)pyridin-2-yl)-[1,1′-biphenyl]-3-yl)-3-(((3-hydroxy-2,2-dimethylpropyl)amino)methyl)-4H-pyrido[1,2-a]pyrimidin-4-one;
3-((((2S)-2-cyclopropyl-2-hydroxyethyl)amino)methyl)-8-(2,2′-dichloro-3′-(6-methoxy-5-(((((S)-5-oxopyrrolidin-2-yl)methyl)amino)methyl)pyridin-2-yl)-[1,1′-biphenyl]-3-yl)-4H-pyrido[1,2-a]pyrimidin-4-one;
3-((((2R)-2-cyclopropyl-2-hydroxyethyl)amino)methyl)-8-(2,2′-dichloro-3′-(6-methoxy-5-(((((S)-5-oxopyrrolidin-2-yl)methyl)amino)methyl)pyridin-2-yl)-[1,1′-biphenyl]-3-yl)-4H-pyrido[1,2-a]pyrimidin-4-one;
3-((((2S)-2-cyclopropyl-2-hydroxyethyl)amino)methyl)-8-(2,2′-dichloro-3′-(6-methoxy-5-(((((R)-5-oxopyrrolidin-2-yl)methyl)amino)methyl)pyridin-2-yl)-[1,1′-biphenyl]-3-yl)-4H-pyrido[1,2-a]pyrimidin-4-one;
3-((((2R)-2-cyclopropyl-2-hydroxyethyl)amino)methyl)-8-(2,2′-dichloro-3′-(6-methoxy-5-(((((R)-5-oxopyrrolidin-2-yl)methyl)amino)methyl)pyridin-2-yl)-[1,1′-biphenyl]-3-yl)-4H-pyrido[1,2-a]pyrimidin-4-one;
(S)-4-(((8-(2,2′-dichloro-3′-(4-fluoro-6-methoxy-5-(((((S)-5-oxopyrrolidin-2-yl)methyl)amino)methyl)pyridin-2-yl)-[1,1′-biphenyl]-3-yl)-4-oxo-4H-pyrido[1,2-a]pyrimidin-3-yl)methyl)amino)-3-hydroxybutanamide;
(R)-4-(((8-(2,2′-dichloro-3′-(4-fluoro-6-methoxy-5-(((((S)-5-oxopyrrolidin-2-yl)methyl)amino)methyl)pyridin-2-yl)-[1,1′-biphenyl]-3-yl)-4-oxo-4H-pyrido[1,2-a]pyrimidin-3-yl)methyl)amino)-3-hydroxybutanamide;
(S)-4-(((8-(2,2′-dichloro-3′-(4-fluoro-6-methoxy-5-(((((R)-5-oxopyrrolidin-2-yl)methyl)amino)methyl)pyridin-2-yl)-[1,1′-biphenyl]-3-yl)-4-oxo-4H-pyrido[1,2-a]pyrimidin-3-yl)methyl)amino)-3-hydroxybutanamide;
(R)-4-(((8-(2,2′-dichloro-3′-(4-fluoro-6-methoxy-5-(((((R)-5-oxopyrrolidin-2-yl)methyl)amino)methyl)pyridin-2-yl)-[1,1′-biphenyl]-3-yl)-4-oxo-4H-pyrido[1,2-a]pyrimidin-3-yl)methyl)amino)-3-hydroxybutanamide;
8-(2,2′-dichloro-3′-(6-methoxy-5-((methyl(3-(methylamino)propyl)amino)methyl)pyridin-2-yl)-[1,1′-biphenyl]-3-yl)-3-((methyl(2-(methylamino)ethyl)amino)methyl)-4H-pyrido[1,2-a]pyrimidin-4-one;
tert-butyl (2-(((6-(3′-(3-(((2-((tert-butoxycarbonyl)amino)ethyl)(methyl)amino)methyl)-4-oxo-4H-pyrido[1,2-a]pyrimidin-8-yl)-2,2′-dichloro-[1,1′-biphenyl]-3-yl)-2-methoxypyridin-3-yl)methyl)(methyl)amino)ethyl)carbamate;
(S)-8-(2,2′-dichloro-3′-(6-methoxy-5-((((5-oxopyrrolidin-2-yl)methyl)amino)methyl)pyridin-2-yl)-[1,1′-biphenyl]-3-yl)-3-((methyl(2-(methylamino)ethyl)amino)methyl)-4H-pyrido[1,2-a]pyrimidin-4-one;
(R)-8-(2,2′-dichloro-3′-(6-methoxy-5-((((5-oxopyrrolidin-2-yl)methyl)amino)methyl)pyridin-2-yl)-[1,1′-biphenyl]-3-yl)-3-((methyl(2-(methylamino)ethyl)amino)methyl)-4H-pyrido[1,2-a]pyrimidin-4-one;
(S)-3-(((2-aminoethyl)(methyl)amino)methyl)-8-(2,2′-dichloro-3′-(6-methoxy-5-((((5-oxopyrrolidin-2-yl)methyl)amino)methyl)pyridin-2-yl)-[1,1′-biphenyl]-3-yl)-4H-pyrido[1,2-a]pyrimidin-4-one;
(R)-3-(((2-aminoethyl)(methyl)amino)methyl)-8-(2,2′-dichloro-3′-(6-methoxy-5-((((5-oxopyrrolidin-2-yl)methyl)amino)methyl)pyridin-2-yl)-[1,1′-biphenyl]-3-yl)-4H-pyrido[1,2-a]pyrimidin-4-one;
3-((((2S)-2-cyclopropyl-2-hydroxypropyl)amino)methyl)-8-(2,2′-dichloro-3′-(6-methoxy-5-(((((S)-5-oxopyrrolidin-2-yl)methyl)amino)methyl)pyridin-2-yl)-[1,1′-biphenyl]-3-yl)-4H-pyrido[1,2-a]pyrimidin-4-one;
3-((((2S)-2-cyclopropyl-2-hydroxypropyl)amino)methyl)-8-(2,2′-dichloro-3′-(6-methoxy-5-(((((R)-5-oxopyrrolidin-2-yl)methyl)amino)methyl)pyridin-2-yl)-[1,1′-biphenyl]-3-yl)-4H-pyrido[1,2-a]pyrimidin-4-one;
3-((((2R)-2-cyclopropyl-2-hydroxypropyl)amino)methyl)-8-(2,2′-dichloro-3′-(6-methoxy-5-(((((S)-5-oxopyrrolidin-2-yl)methyl)amino)methyl)pyridin-2-yl)-[1,1′-biphenyl]-3-yl)-4H-pyrido[1,2-a]pyrimidin-4-one;
3-((((2R)-2-cyclopropyl-2-hydroxypropyl)amino)methyl)-8-(2,2′-dichloro-3′-(6-methoxy-5-(((((R)-5-oxopyrrolidin-2-yl)methyl)amino)methyl)pyridin-2-yl)-[1,1′-biphenyl]-3-yl)-4H-pyrido[1,2-a]pyrimidin-4-one;
(S)-8-(2,2′-dichloro-3′-(6-methoxy-5-((((5-oxopyrrolidin-2-yl)methyl)amino)methyl)pyridin-2-yl)-[1,1′-biphenyl]-3-yl)-3-(((2-hydroxyethyl)(methyl)amino)methyl)-4H-pyrido[1,2-a]pyrimidin-4-one;
(R)-8-(2,2′-dichloro-3′-(6-methoxy-5-((((5-oxopyrrolidin-2-yl)methyl)amino)methyl)pyridin-2-yl)-[1,1′-biphenyl]-3-yl)-3-(((2-hydroxyethyl)(methyl)amino)methyl)-4H-pyrido[1,2-a]pyrimidin-4-one;
(S)-5-((((6-(2,2′-dichloro-3′-(3-(((((S)-5-oxopyrrolidin-2-yl)methyl)amino)methyl)pyrazolo[1,5-a]pyrimidin-5-yl)-[1,1′-biphenyl]-3-yl)-2-methoxypyridin-3-yl)methyl)amino)methyl)pyrrolidin-2-one;
(R)-5-((((6-(2,2′-dichloro-3′-(3-(((((S)-5-oxopyrrolidin-2-yl)methyl)amino)methyl)pyrazolo[1,5-a]pyrimidin-5-yl)-[1,1′-biphenyl]-3-yl)-2-methoxypyridin-3-yl)methyl)amino)methyl)pyrrolidin-2-one;
(S)-5-((((6-(2,2′-dichloro-3′-(3-(((((R)-5-oxopyrrolidin-2-yl)methyl)amino)methyl)pyrazolo[1,5-a]pyrimidin-5-yl)-[1,1′-biphenyl]-3-yl)-2-methoxypyridin-3-yl)methyl)amino)methyl)pyrrolidin-2-one;
(R)-5-((((6-(2,2′-dichloro-3′-(3-(((((R)-5-oxopyrrolidin-2-yl)methyl)amino)methyl)pyrazolo[1,5-a]pyrimidin-5-yl)-[1,1′-biphenyl]-3-yl)-2-methoxypyridin-3-yl)methyl)amino)methyl)pyrrolidin-2-one;
(S)-8-(3′-(5-(1-(aminomethyl)cyclopropyl)-6-methoxypyridin-2-yl)-2,2′-dichloro-[1,1′-biphenyl]-3-yl)-3-((((5-oxopyrrolidin-2-yl)methyl)amino)methyl)-4H-pyrido[1,2-a]pyrimidin-4-one;
(R)-8-(3′-(5-(1-(aminomethyl)cyclopropyl)-6-methoxypyridin-2-yl)-2,2′-dichloro-[1,1′-biphenyl]-3-yl)-3-((((5-oxopyrrolidin-2-yl)methyl)amino)methyl)-4H-pyrido[1,2-a]pyrimidin-4-one;
8-(2,2′-dichloro-3′-(6-methoxy-5-(((((S)-5-oxopyrrolidin-2-yl)methyl)amino)methyl)pyridin-2-yl)-[1,1′-biphenyl]-3-yl)-3-(((((S)-5-oxopyrrolidin-3-yl)methyl)amino)methyl)-4H-pyrido[1,2-a]pyrimidin-4-one;
8-(2,2′-dichloro-3′-(6-methoxy-5-(((((R)-5-oxopyrrolidin-2-yl)methyl)amino)methyl)pyridin-2-yl)-[1,1′-biphenyl]-3-yl)-3-(((((S)-5-oxopyrrolidin-3-yl)methyl)amino)methyl)-4H-pyrido[1,2-a]pyrimidin-4-one;
8-(2,2′-dichloro-3′-(6-methoxy-5-(((((S)-5-oxopyrrolidin-2-yl)methyl)amino)methyl)pyridin-2-yl)-[1,1′-biphenyl]-3-yl)-3-(((((R)-5-oxopyrrolidin-3-yl)methyl)amino)methyl)-4H-pyrido[1,2-a]pyrimidin-4-one;
8-(2,2′-dichloro-3′-(6-methoxy-5-(((((R)-5-oxopyrrolidin-2-yl)methyl)amino)methyl)pyridin-2-yl)-[1,1′-biphenyl]-3-yl)-3-(((((R)-5-oxopyrrolidin-3-yl)methyl)amino)methyl)-4H-pyrido[1,2-a]pyrimidin-4-one;
8-(2,2′-dichloro-3′-(6-methoxy-5-(((((S)-5-oxopyrrolidin-2-yl)methyl)amino)methyl)pyridin-2-yl)-[1,1′-biphenyl]-3-yl)-3-((((3R,4S)-3-hydroxytetrahydro-2H-pyran-4-yl)amino)methyl)-2-methyl-4H-pyrido[1,2-a]pyrimidin-4-one;
8-(2,2′-dichloro-3′-(6-methoxy-5-(((((S)-5-oxopyrrolidin-2-yl)methyl)amino)methyl)pyridin-2-yl)-[1,1′-biphenyl]-3-yl)-3-((((3S,4S)-3-hydroxytetrahydro-2H-pyran-4-yl)amino)methyl)-2-methyl-4H-pyrido[1,2-a]pyrimidin-4-one;
8-(2,2′-dichloro-3′-(6-methoxy-5-(((((S)-5-oxopyrrolidin-2-yl)methyl)amino)methyl)pyridin-2-yl)-[1,1′-biphenyl]-3-yl)-3-((((3R,4R)-3-hydroxytetrahydro-2H-pyran-4-yl)amino)methyl)-2-methyl-4H-pyrido[1,2-a]pyrimidin-4-one;
8-(2,2′-dichloro-3′-(6-methoxy-5-(((((S)-5-oxopyrrolidin-2-yl)methyl)amino)methyl)pyridin-2-yl)-[1,1′-biphenyl]-3-yl)-3-((((3S,4R)-3-hydroxytetrahydro-2H-pyran-4-yl)amino)methyl)-2-methyl-4H-pyrido[1,2-a]pyrimidin-4-one;
8-(2,2′-dichloro-3′-(6-methoxy-5-(((((R)-5-oxopyrrolidin-2-yl)methyl)amino)methyl)pyridin-2-yl)-[1,1′-biphenyl]-3-yl)-3-((((3R,4S)-3-hydroxytetrahydro-2H-pyran-4-yl)amino)methyl)-2-methyl-4H-pyrido[1,2-a]pyrimidin-4-one;
8-(2,2′-dichloro-3′-(6-methoxy-5-(((((R)-5-oxopyrrolidin-2-yl)methyl)amino)methyl)pyridin-2-yl)-[1,1′-biphenyl]-3-yl)-3-((((3S,4S)-3-hydroxytetrahydro-2H-pyran-4-yl)amino)methyl)-2-methyl-4H-pyrido[1,2-a]pyrimidin-4-one;
8-(2,2′-dichloro-3′-(6-methoxy-5-(((((R)-5-oxopyrrolidin-2-yl)methyl)amino)methyl)pyridin-2-yl)-[1,1′-biphenyl]-3-yl)-3-((((3R,4R)-3-hydroxytetrahydro-2H-pyran-4-yl)amino)methyl)-2-methyl-4H-pyrido[1,2-a]pyrimidin-4-one;
8-(2,2′-dichloro-3′-(6-methoxy-5-(((((R)-5-oxopyrrolidin-2-yl)methyl)amino)methyl)pyridin-2-yl)-[1,1′-biphenyl]-3-yl)-3-((((3S,4R)-3-hydroxytetrahydro-2H-pyran-4-yl)amino)methyl)-2-methyl-4H-pyrido[1,2-a]pyrimidin-4-one;
8-(2,2′-dichloro-3′-(5-((((3R,4R)-3-hydroxytetrahydro-2H-pyran-4-yl)amino)methyl)-6-methoxypyridin-2-yl)-[1,1′-biphenyl]-3-yl)-2-methyl-3-(((((S)-5-oxopyrrolidin-2-yl)methyl)amino)methyl)-4H-pyrido[1,2-a]pyrimidin-4-one;
8-(2,2′-dichloro-3′-(5-((((3R,4S)-3-hydroxytetrahydro-2H-pyran-4-yl)amino)methyl)-6-methoxypyridin-2-yl)-[1,1′-biphenyl]-3-yl)-2-methyl-3-(((((S)-5-oxopyrrolidin-2-yl)methyl)amino)methyl)-4H-pyrido[1,2-a]pyrimidin-4-one;
8-(2,2′-dichloro-3′-(5-((((3S,4R)-3-hydroxytetrahydro-2H-pyran-4-yl)amino)methyl)-6-methoxypyridin-2-yl)-[1,1′-biphenyl]-3-yl)-2-methyl-3-(((((S)-5-oxopyrrolidin-2-yl)methyl)amino)methyl)-4H-pyrido[1,2-a]pyrimidin-4-one;
8-(2,2′-dichloro-3′-(5-((((3S,4S)-3-hydroxytetrahydro-2H-pyran-4-yl)amino)methyl)-6-methoxypyridin-2-yl)-[1,1′-biphenyl]-3-yl)-2-methyl-3-(((((S)-5-oxopyrrolidin-2-yl)methyl)amino)methyl)-4H-pyrido[1,2-a]pyrimidin-4-one;
8-(2,2′-dichloro-3′-(5-((((3R,4R)-3-hydroxytetrahydro-2H-pyran-4-yl)amino)methyl)-6-methoxypyridin-2-yl)-[1,1′-biphenyl]-3-yl)-2-methyl-3-(((((R)-5-oxopyrrolidin-2-yl)methyl)amino)methyl)-4H-pyrido[1,2-a]pyrimidin-4-one;
8-(2,2′-dichloro-3′-(5-((((3R,4S)-3-hydroxytetrahydro-2H-pyran-4-yl)amino)methyl)-6-methoxypyridin-2-yl)-[1,1′-biphenyl]-3-yl)-2-methyl-3-(((((R)-5-oxopyrrolidin-2-yl)methyl)amino)methyl)-4H-pyrido[1,2-a]pyrimidin-4-one;
8-(2,2′-dichloro-3′-(5-((((3S,4R)-3-hydroxytetrahydro-2H-pyran-4-yl)amino)methyl)-6-methoxypyridin-2-yl)-[1,1′-biphenyl]-3-yl)-2-methyl-3-(((((R)-5-oxopyrrolidin-2-yl)methyl)amino)methyl)-4H-pyrido[1,2-a]pyrimidin-4-one;
8-(2,2′-dichloro-3′-(5-((((3S,4S)-3-hydroxytetrahydro-2H-pyran-4-yl)amino)methyl)-6-methoxypyridin-2-yl)-[1,1′-biphenyl]-3-yl)-2-methyl-3-(((((R)-5-oxopyrrolidin-2-yl)methyl)amino)methyl)-4H-pyrido[1,2-a]pyrimidin-4-one;
(S)-8-(2,2′-dichloro-3′-(6-methoxy-5-((((5-oxopyrrolidin-2-yl)methyl)amino)methyl)pyridin-2-yl)-[1,1′-biphenyl]-3-yl)-3-(((2-hydroxyethyl)amino)methyl)-2-methyl-4H-pyrido[1,2-a]pyrimidin-4-one;
(R)-8-(2,2′-dichloro-3′-(6-methoxy-5-((((5-oxopyrrolidin-2-yl)methyl)amino)methyl)pyridin-2-yl)-[1,1′-biphenyl]-3-yl)-3-(((2-hydroxyethyl)amino)methyl)-2-methyl-4H-pyrido[1,2-a]pyrimidin-4-one;
7-(2,2′-dichloro-3′-(6-methoxy-5-(((((S)-5-oxopyrrolidin-2-yl)methyl)amino)methyl)pyridin-2-yl)-[1,1′-biphenyl]-3-yl)-9-methyl-2-(((((S)-5-oxopyrrolidin-2-yl)methyl)amino)methyl)-4H-pyrido[1,2-a]pyrimidin-4-one;
7-(2,2′-dichloro-3′-(6-methoxy-5-(((((R)-5-oxopyrrolidin-2-yl)methyl)amino)methyl)pyridin-2-yl)-[1,1′-biphenyl]-3-yl)-9-methyl-2-(((((S)-5-oxopyrrolidin-2-yl)methyl)amino)methyl)-4H-pyrido[1,2-a]pyrimidin-4-one;
7-(2,2′-dichloro-3′-(6-methoxy-5-(((((S)-5-oxopyrrolidin-2-yl)methyl)amino)methyl)pyridin-2-yl)-[1,1′-biphenyl]-3-yl)-9-methyl-2-(((((R)-5-oxopyrrolidin-2-yl)methyl)amino)methyl)-4H-pyrido[1,2-a]pyrimidin-4-one;
7-(2,2′-dichloro-3′-(6-methoxy-5-(((((R)-5-oxopyrrolidin-2-yl)methyl)amino)methyl)pyridin-2-yl)-[1,1′-biphenyl]-3-yl)-9-methyl-2-(((((R)-5-oxopyrrolidin-2-yl)methyl)amino)methyl)-4H-pyrido[1,2-a]pyrimidin-4-one;
methyl 1-((6-(2,2′-dichloro-3′-(3-((3-(methoxycarbonyl)azetidin-1-yl)methyl)-1,6-naphthyridin-7-yl)-[1,1′-biphenyl]-3-yl)-2-methoxypyridin-3-yl)methyl)azetidine-3-carboxylate;
8-(2,2′-dichloro-3′-(6-methoxy-5-(((((S)-5-oxopyrrolidin-2-yl)methyl)amino)methyl)pyridin-2-yl)-[1,1′-biphenyl]-3-yl)-3-(5-hydroxy-1,4,5,6-tetrahydropyrimidin-2-yl)-2-methyl-4H-pyrido[1,2-a]pyrimidin-4-one;
8-(2,2′-dichloro-3′-(6-methoxy-5-(((((R)-5-oxopyrrolidin-2-yl)methyl)amino)methyl)pyridin-2-yl)-[1,1′-biphenyl]-3-yl)-3-(5-hydroxy-1,4,5,6-tetrahydropyrimidin-2-yl)-2-methyl-4H-pyrido[1,2-a]pyrimidin-4-one;
methyl (S)-1-((8-(2,2′-dichloro-3′-(6-methoxy-5-((((5-oxopyrrolidin-2-yl)methyl)amino)methyl)pyridin-2-yl)-[1,1′-biphenyl]-3-yl)-6-methyl-4-oxo-4H-pyrido[1,2-a]pyrimidin-3-yl)methyl)azetidine-3-carboxylate;
methyl (R)-1-((8-(2,2′-dichloro-3′-(6-methoxy-5-((((5-oxopyrrolidin-2-yl)methyl)amino)methyl)pyridin-2-yl)-[1,1′-biphenyl]-3-yl)-6-methyl-4-oxo-4H-pyrido[1,2-a]pyrimidin-3-yl)methyl)azetidine-3-carboxylate;
(S)-4-(((8-(2,2′-dichloro-3′-(6-methoxy-5-(((((S)-5-oxopyrrolidin-2-yl)methyl)amino)methyl)pyridin-2-yl)-[1,1′-biphenyl]-3-yl)-4-oxo-4H-pyrido[1,2-a]pyrimidin-3-yl)methyl)amino)-3-hydroxy-N-methylbutanamide;
(R)-4-(((8-(2,2′-dichloro-3′-(6-methoxy-5-(((((S)-5-oxopyrrolidin-2-yl)methyl)amino)methyl)pyridin-2-yl)-[1,1′-biphenyl]-3-yl)-4-oxo-4H-pyrido[1,2-a]pyrimidin-3-yl)methyl)amino)-3-hydroxy-N-methylbutanamide;
(S)-4-(((8-(2,2′-dichloro-3′-(6-methoxy-5-(((((R)-5-oxopyrrolidin-2-yl)methyl)amino)methyl)pyridin-2-yl)-[1,1′-biphenyl]-3-yl)-4-oxo-4H-pyrido[1,2-a]pyrimidin-3-yl)methyl)amino)-3-hydroxy-N-methylbutanamide;
(R)-4-(((8-(2,2′-dichloro-3′-(6-methoxy-5-(((((R)-5-oxopyrrolidin-2-yl)methyl)amino)methyl)pyridin-2-yl)-[1,1′-biphenyl]-3-yl)-4-oxo-4H-pyrido[1,2-a]pyrimidin-3-yl)methyl)amino)-3-hydroxy-N-methylbutanamide;
(S)-4-(((8-(2,2′-dichloro-3′-(4-fluoro-6-methoxy-5-(((((S)-5-oxopyrrolidin-2-yl)methyl)amino)methyl)pyridin-2-yl)-[1,1′-biphenyl]-3-yl)-4-oxo-4H-pyrido[1,2-a]pyrimidin-3-yl)methyl)amino)-3-hydroxy-N-methylbutanamide;
(R)-4-(((8-(2,2′-dichloro-3′-(4-fluoro-6-methoxy-5-(((((S)-5-oxopyrrolidin-2-yl)methyl)amino)methyl)pyridin-2-yl)-[1,1′-biphenyl]-3-yl)-4-oxo-4H-pyrido[1,2-a]pyrimidin-3-yl)methyl)amino)-3-hydroxy-N-methylbutanamide;
(S)-4-(((8-(2,2′-dichloro-3′-(4-fluoro-6-methoxy-5-(((((R)-5-oxopyrrolidin-2-yl)methyl)amino)methyl)pyridin-2-yl)-[1,1′-biphenyl]-3-yl)-4-oxo-4H-pyrido[1,2-a]pyrimidin-3-yl)methyl)amino)-3-hydroxy-N-methylbutanamide;
(R)-4-(((8-(2,2′-dichloro-3′-(4-fluoro-6-methoxy-5-(((((R)-5-oxopyrrolidin-2-yl)methyl)amino)methyl)pyridin-2-yl)-[1,1′-biphenyl]-3-yl)-4-oxo-4H-pyrido[1,2-a]pyrimidin-3-yl)methyl)amino)-3-hydroxy-N-methylbutanamide;
methyl (S)-((8-(2,2′-dichloro-3′-(6-methoxy-5-((((5-oxopyrrolidin-2-yl)methyl)amino)methyl)pyridin-2-yl)-[1,1′-biphenyl]-3-yl)-6-methyl-4-oxo-4H-pyrido[1,2-a]pyrimidin-3-yl)methyl)glycinate;
methyl (R)-((8-(2,2′-dichloro-3′-(6-methoxy-5-((((5-oxopyrrolidin-2-yl)methyl)amino)methyl)pyridin-2-yl)-[1,1′-biphenyl]-3-yl)-6-methyl-4-oxo-4H-pyrido[1,2-a]pyrimidin-3-yl)methyl)glycinate;
methyl (S)-1-((8-(2,2′-dichloro-3′-(6-methoxy-5-(((((S)-5-oxopyrrolidin-2-yl)methyl)amino)methyl)pyridin-2-yl)-[1,1′-biphenyl]-3-yl)-6-methyl-4-oxo-4H-pyrido[1,2-a]pyrimidin-3-yl)methyl)pyrrolidine-3-carboxylate;
methyl (R)-1-((8-(2,2′-dichloro-3′-(6-methoxy-5-(((((S)-5-oxopyrrolidin-2-yl)methyl)amino)methyl)pyridin-2-yl)-[1,1′-biphenyl]-3-yl)-6-methyl-4-oxo-4H-pyrido[1,2-a]pyrimidin-3-yl)methyl)pyrrolidine-3-carboxylate;
methyl (S)-1-((8-(2,2′-dichloro-3′-(6-methoxy-5-(((((R)-5-oxopyrrolidin-2-yl)methyl)amino)methyl)pyridin-2-yl)-[1,1′-biphenyl]-3-yl)-6-methyl-4-oxo-4H-pyrido[1,2-a]pyrimidin-3-yl)methyl)pyrrolidine-3-carboxylate;
methyl (R)-1-((8-(2,2′-dichloro-3′-(6-methoxy-5-(((((R)-5-oxopyrrolidin-2-yl)methyl)amino)methyl)pyridin-2-yl)-[1,1′-biphenyl]-3-yl)-6-methyl-4-oxo-4H-pyrido[1,2-a]pyrimidin-3-yl)methyl)pyrrolidine-3-carboxylate;
methyl (S)-1-((8-(2,2′-dichloro-3′-(6-methoxy-5-((((5-oxopyrrolidin-2-yl)methyl)amino)methyl)pyridin-2-yl)-[1,1′-biphenyl]-3-yl)-2-methyl-4-oxo-4H-pyrido[1,2-a]pyrimidin-3-yl)methyl)azetidine-3-carboxylate;
methyl (R)-1-((8-(2,2′-dichloro-3′-(6-methoxy-5-((((5-oxopyrrolidin-2-yl)methyl)amino)methyl)pyridin-2-yl)-[1,1′-biphenyl]-3-yl)-2-methyl-4-oxo-4H-pyrido[1,2-a]pyrimidin-3-yl)methyl)azetidine-3-carboxylate;
(S)-8-(2,2′-dichloro-3′-(6-methoxy-5-((((5-oxopyrrolidin-2-yl)methyl)amino)methyl)pyridin-2-yl)-[1,1′-biphenyl]-3-yl)-3-(((2,2-difluoroethyl)amino)methyl)-4H-pyrido[1,2-a]pyrimidin-4-one;
(R)-8-(2,2′-dichloro-3′-(6-methoxy-5-((((5-oxopyrrolidin-2-yl)methyl)amino)methyl)pyridin-2-yl)-[1,1′-biphenyl]-3-yl)-3-(((2,2-difluoroethyl)amino)methyl)-4H-pyrido[1,2-a]pyrimidin-4-one;
3-((((S)-2-aminopropyl)amino)methyl)-8-(2,2′-dichloro-3′-(6-methoxy-5-(((((S)-5-oxopyrrolidin-2-yl)methyl)amino)methyl)pyridin-2-yl)-[1,1′-biphenyl]-3-yl)-4H-pyrido[1,2-a]pyrimidin-4-one;
3-((((R)-2-aminopropyl)amino)methyl)-8-(2,2′-dichloro-3′-(6-methoxy-5-(((((S)-5-oxopyrrolidin-2-yl)methyl)amino)methyl)pyridin-2-yl)-[1,1′-biphenyl]-3-yl)-4H-pyrido[1,2-a]pyrimidin-4-one;
3-((((S)-2-aminopropyl)amino)methyl)-8-(2,2′-dichloro-3′-(6-methoxy-5-(((((R)-5-oxopyrrolidin-2-yl)methyl)amino)methyl)pyridin-2-yl)-[1,1′-biphenyl]-3-yl)-4H-pyrido[1,2-a]pyrimidin-4-one;
3-((((R)-2-aminopropyl)amino)methyl)-8-(2,2′-dichloro-3′-(6-methoxy-5-(((((R)-5-oxopyrrolidin-2-yl)methyl)amino)methyl)pyridin-2-yl)-[1,1′-biphenyl]-3-yl)-4H-pyrido[1,2-a]pyrimidin-4-one;
methyl (2-(((8-(2,2′-dichloro-3′-(6-methoxy-5-(((2-((methoxycarbonyl)(methyl)amino)ethyl)(methyl)amino)methyl)pyridin-2-yl)-[1,1′-biphenyl]-3-yl)-4-oxo-4H-pyrido[1,2-a]pyrimidin-3-yl)methyl)(methyl)amino)ethyl)(methyl)carbamate;
(S)-8-(2,2′-dichloro-3′-(6-methoxy-5-((((5-oxopyrrolidin-2-yl)methyl)amino)methyl)pyridin-2-yl)-[1,1′-biphenyl]-3-yl)-3-(((2,2-difluoro-3-hydroxypropyl)amino)methyl)-4H-pyrido[1,2-a]pyrimidin-4-one;
(R)-8-(2,2′-dichloro-3′-(6-methoxy-5-((((5-oxopyrrolidin-2-yl)methyl)amino)methyl)pyridin-2-yl)-[1,1′-biphenyl]-3-yl)-3-(((2,2-difluoro-3-hydroxypropyl)amino)methyl)-4H-pyrido[1,2-a]pyrimidin-4-one;
(S)-8-(2,2′-dichloro-3′-(6-methoxy-5-((((5-oxopyrrolidin-2-yl)methyl)amino)methyl)pyridin-2-yl)-[1,1′-biphenyl]-3-yl)-3-((((1-(hydroxymethyl)cyclopropyl)methyl)amino)methyl)-4H-pyrido[1,2-a]pyrimidin-4-one;
(R)-8-(2,2′-dichloro-3′-(6-methoxy-5-((((5-oxopyrrolidin-2-yl)methyl)amino)methyl)pyridin-2-yl)-[1,1′-biphenyl]-3-yl)-3-((((1-(hydroxymethyl)cyclopropyl)methyl)amino)methyl)-4H-pyrido[1,2-a]pyrimidin-4-one;
8-(2,2′-dichloro-3′-(6-methoxy-5-(((((S)-5-oxopyrrolidin-2-yl)methyl)amino)methyl)pyridin-2-yl)-[1,1′-biphenyl]-3-yl)-3-((((S)-2-methoxypropyl)amino)methyl)-4H-pyrido[1,2-a]pyrimidin-4-one;
8-(2,2′-dichloro-3′-(6-methoxy-5-(((((R)-5-oxopyrrolidin-2-yl)methyl)amino)methyl)pyridin-2-yl)-[1,1′-biphenyl]-3-yl)-3-((((S)-2-methoxypropyl)amino)methyl)-4H-pyrido[1,2-a]pyrimidin-4-one;
8-(2,2′-dichloro-3′-(6-methoxy-5-(((((S)-5-oxopyrrolidin-2-yl)methyl)amino)methyl)pyridin-2-yl)-[1,1′-biphenyl]-3-yl)-3-((((R)-2-methoxypropyl)amino)methyl)-4H-pyrido[1,2-a]pyrimidin-4-one;
8-(2,2′-dichloro-3′-(6-methoxy-5-(((((R)-5-oxopyrrolidin-2-yl)methyl)amino)methyl)pyridin-2-yl)-[1,1′-biphenyl]-3-yl)-3-((((R)-2-methoxypropyl)amino)methyl)-4H-pyrido[1,2-a]pyrimidin-4-one;
(S)-8-(2,2′-dichloro-3′-(6-methoxy-5-((((5-oxopyrrolidin-2-yl)methyl)amino)methyl)pyridin-2-yl)-[1,1′-biphenyl]-3-yl)-3-(((2,2,2-trifluoroethyl)amino)methyl)-4H-pyrido[1,2-a]pyrimidin-4-one;
(R)-8-(2,2′-dichloro-3′-(6-methoxy-5-((((5-oxopyrrolidin-2-yl)methyl)amino)methyl)pyridin-2-yl)-[1,1′-biphenyl]-3-yl)-3-(((2,2,2-trifluoroethyl)amino)methyl)-4H-pyrido[1,2-a]pyrimidin-4-one;
methyl (S)-1-((7-(2,2′-dichloro-3′-(6-methoxy-5-((((5-oxopyrrolidin-2-yl)methyl)amino)methyl)pyridin-2-yl)-[1,1′-biphenyl]-3-yl)-1,6-naphthyridin-3-yl)methyl)azetidine-3-carboxylate;
methyl (R)-1-((7-(2,2′-dichloro-3′-(6-methoxy-5-((((5-oxopyrrolidin-2-yl)methyl)amino)methyl)pyridin-2-yl)-[1,1′-biphenyl]-3-yl)-1,6-naphthyridin-3-yl)methyl)azetidine-3-carboxylate;
methyl (S)-((7-(2,2′-dichloro-3′-(6-methoxy-5-((((5-oxopyrrolidin-2-yl)methyl)amino)methyl)pyridin-2-yl)-[1,1′-biphenyl]-3-yl)-1,6-naphthyridin-3-yl)methyl)glycinate;
methyl (R)-((7-(2,2′-dichloro-3′-(6-methoxy-5-((((5-oxopyrrolidin-2-yl)methyl)amino)methyl)pyridin-2-yl)-[1,1′-biphenyl]-3-yl)-1,6-naphthyridin-3-yl)methyl)glycinate;
8-(2,2′-dichloro-3′-(6-methoxy-5-(((((S)-5-oxopyrrolidin-2-yl)methyl)amino)methyl)pyridin-2-yl)-[1,1′-biphenyl]-3-yl)-3-(((((S)-oxetan-2-yl)methyl)amino)methyl)-4H-pyrido[1,2-a]pyrimidin-4-one;
8-(2,2′-dichloro-3′-(6-methoxy-5-(((((R)-5-oxopyrrolidin-2-yl)methyl)amino)methyl)pyridin-2-yl)-[1,1′-biphenyl]-3-yl)-3-(((((S)-oxetan-2-yl)methyl)amino)methyl)-4H-pyrido[1,2-a]pyrimidin-4-one;
8-(2,2′-dichloro-3′-(6-methoxy-5-(((((S)-5-oxopyrrolidin-2-yl)methyl)amino)methyl)pyridin-2-yl)-[1,1′-biphenyl]-3-yl)-3-(((((R)-oxetan-2-yl)methyl)amino)methyl)-4H-pyrido[1,2-a]pyrimidin-4-one;
8-(2,2′-dichloro-3′-(6-methoxy-5-(((((R)-5-oxopyrrolidin-2-yl)methyl)amino)methyl)pyridin-2-yl)-[1,1′-biphenyl]-3-yl)-3-(((((R)-oxetan-2-yl)methyl)amino)methyl)-4H-pyrido[1,2-a]pyrimidin-4-one;
8-(2,2′-dichloro-3′-(6-methoxy-5-(((((S)-5-oxopyrrolidin-2-yl)methyl)amino)methyl)pyridin-2-yl)-[1,1′-biphenyl]-3-yl)-3-(((((S)-tetrahydrofuran-2-yl)methyl)amino)methyl)-4H-pyrido[1,2-a]pyrimidin-4-one;
8-(2,2′-dichloro-3′-(6-methoxy-5-(((((R)-5-oxopyrrolidin-2-yl)methyl)amino)methyl)pyridin-2-yl)-[1,1′-biphenyl]-3-yl)-3-(((((S)-tetrahydrofuran-2-yl)methyl)amino)methyl)-4H-pyrido[1,2-a]pyrimidin-4-one;
8-(2,2′-dichloro-3′-(6-methoxy-5-(((((S)-5-oxopyrrolidin-2-yl)methyl)amino)methyl)pyridin-2-yl)-[1,1′-biphenyl]-3-yl)-3-(((((R)-tetrahydrofuran-2-yl)methyl)amino)methyl)-4H-pyrido[1,2-a]pyrimidin-4-one;
8-(2,2′-dichloro-3′-(6-methoxy-5-(((((R)-5-oxopyrrolidin-2-yl)methyl)amino)methyl)pyridin-2-yl)-[1,1′-biphenyl]-3-yl)-3-(((((R)-tetrahydrofuran-2-yl)methyl)amino)methyl)-4H-pyrido[1,2-a]pyrimidin-4-one;
(S)-8-(2,2′-dichloro-3′-(6-methoxy-5-((((5-oxopyrrolidin-2-yl)methyl)amino)methyl)pyridin-2-yl)-[1,1′-biphenyl]-3-yl)-3-(((2-hydroxyethyl)amino)methyl)-6-methyl-4H-pyrido[1,2-a]pyrimidin-4-one;
(R)-8-(2,2′-dichloro-3′-(6-methoxy-5-((((5-oxopyrrolidin-2-yl)methyl)amino)methyl)pyridin-2-yl)-[1,1′-biphenyl]-3-yl)-3-(((2-hydroxyethyl)amino)methyl)-6-methyl-4H-pyrido[1,2-a]pyrimidin-4-one;
(S)-5-((((6-(2,2′-dichloro-3′-(3-(((((S)-5-oxopyrrolidin-2-yl)methyl)amino)methyl)imidazo[1,2-a]pyrazin-6-yl)-[1,1′-biphenyl]-3-yl)-2-methoxypyridin-3-yl)methyl)amino)methyl)pyrrolidin-2-one;
(R)-5-((((6-(2,2′-dichloro-3′-(3-(((((S)-5-oxopyrrolidin-2-yl)methyl)amino)methyl)imidazo[1,2-a]pyrazin-6-yl)-[1,1′-biphenyl]-3-yl)-2-methoxypyridin-3-yl)methyl)amino)methyl)pyrrolidin-2-one;
(S)-5-((((6-(2,2′-dichloro-3′-(3-(((((R)-5-oxopyrrolidin-2-yl)methyl)amino)methyl)imidazo[1,2-a]pyrazin-6-yl)-[1,1′-biphenyl]-3-yl)-2-methoxypyridin-3-yl)methyl)amino)methyl)pyrrolidin-2-one;
(R)-5-((((6-(2,2′-dichloro-3′-(3-(((((R)-5-oxopyrrolidin-2-yl)methyl)amino)methyl)imidazo[1,2-a]pyrazin-6-yl)-[1,1′-biphenyl]-3-yl)-2-methoxypyridin-3-yl)methyl)amino)methyl)pyrrolidin-2-one;
(S)-5-((((6-(2,2′-dichloro-3′-(3-(((((S)-5-oxopyrrolidin-2-yl)methyl)amino)methyl)pyrazolo[1,5-a]pyrimidin-6-yl)-[1,1′-biphenyl]-3-yl)-2-methoxypyridin-3-yl)methyl)amino)methyl)pyrrolidin-2-one;
(R)-5-((((6-(2,2′-dichloro-3′-(3-(((((S)-5-oxopyrrolidin-2-yl)methyl)amino)methyl)pyrazolo[1,5-a]pyrimidin-6-yl)-[1,1′-biphenyl]-3-yl)-2-methoxypyridin-3-yl)methyl)amino)methyl)pyrrolidin-2-one;
(S)-5-((((6-(2,2′-dichloro-3′-(3-(((((R)-5-oxopyrrolidin-2-yl)methyl)amino)methyl)pyrazolo[1,5-a]pyrimidin-6-yl)-[1,1′-biphenyl]-3-yl)-2-methoxypyridin-3-yl)methyl)amino)methyl)pyrrolidin-2-one;
(R)-5-((((6-(2,2′-dichloro-3′-(3-(((((R)-5-oxopyrrolidin-2-yl)methyl)amino)methyl)pyrazolo[1,5-a]pyrimidin-6-yl)-[1,1′-biphenyl]-3-yl)-2-methoxypyridin-3-yl)methyl)amino)methyl)pyrrolidin-2-one;
(S)-5-((((6-(2,2′-dichloro-3′-(2-(((((S)-5-oxopyrrolidin-2-yl)methyl)amino)methyl)furo[3,2-b]pyridin-6-yl)-[1,1′-biphenyl]-3-yl)-2-methoxypyridin-3-yl)methyl)amino)methyl)pyrrolidin-2-one;
(R)-5-((((6-(2,2′-dichloro-3′-(2-(((((S)-5-oxopyrrolidin-2-yl)methyl)amino)methyl)furo[3,2-b]pyridin-6-yl)-[1,1′-biphenyl]-3-yl)-2-methoxypyridin-3-yl)methyl)amino)methyl)pyrrolidin-2-one;
(S)-5-((((6-(2,2′-dichloro-3′-(2-(((((R)-5-oxopyrrolidin-2-yl)methyl)amino)methyl)furo[3,2-b]pyridin-6-yl)-[1,1′-biphenyl]-3-yl)-2-methoxypyridin-3-yl)methyl)amino)methyl)pyrrolidin-2-one;
(R)-5-((((6-(2,2′-dichloro-3′-(2-(((((R)-5-oxopyrrolidin-2-yl)methyl)amino)methyl)furo[3,2-b]pyridin-6-yl)-[1,1′-biphenyl]-3-yl)-2-methoxypyridin-3-yl)methyl)amino)methyl)pyrrolidin-2-one;
(S)-5-((((6-(2,2′-dichloro-3′-(1-methyl-2-(((((S)-5-oxopyrrolidin-2-yl)methyl)amino)methyl)-1H-imidazo[4,5-b]pyridin-5-yl)-[1,1′-biphenyl]-3-yl)-2-methoxypyridin-3-yl)methyl)amino)methyl)pyrrolidin-2-one;
(R)-5-((((6-(2,2′-dichloro-3′-(1-methyl-2-(((((S)-5-oxopyrrolidin-2-yl)methyl)amino)methyl)-1H-imidazo[4,5-b]pyridin-5-yl)-[1,1′-biphenyl]-3-yl)-2-methoxypyridin-3-yl)methyl)amino)methyl)pyrrolidin-2-one;
(S)-5-((((6-(2,2′-dichloro-3′-(1-methyl-2-(((((R)-5-oxopyrrolidin-2-yl)methyl)amino)methyl)-1H-imidazo[4,5-b]pyridin-5-yl)-[1,1′-biphenyl]-3-yl)-2-methoxypyridin-3-yl)methyl)amino)methyl)pyrrolidin-2-one;
(R)-5-((((6-(2,2′-dichloro-3′-(1-methyl-2-(((((R)-5-oxopyrrolidin-2-yl)methyl)amino)methyl)-1H-imidazo[4,5-b]pyridin-5-yl)-[1,1′-biphenyl]-3-yl)-2-methoxypyridin-3-yl)methyl)amino)methyl)pyrrolidin-2-one;
(1r,3r)-3-(((6-(2,2′-dichloro-3′-(2-((((1r,3r)-3-hydroxycyclobutyl)amino)methyl)-1-methyl-1H-imidazo[4,5-b]pyridin-5-yl)-[1,1′-biphenyl]-3-yl)-2-methoxypyridin-3-10 yl)methyl)amino)cyclobutan-1-ol;
(1s,3s)-3-(((6-(2,2′-dichloro-3′-(2-((((1r,3r)-3-hydroxycyclobutyl)amino)methyl)-1-methyl-1H-imidazo[4,5-b]pyridin-5-yl)-[1,1′-biphenyl]-3-yl)-2-methoxypyridin-3-yl)methyl)amino)cyclobutan-1-ol;
(1r,3r)-3-(((6-(2,2′-dichloro-3′-(2-((((1s,3s)-3-hydroxycyclobutyl)amino)methyl)-1-methyl-1H-imidazo[4,5-b]pyridin-5-yl)-[1,1′-biphenyl]-3-yl)-2-methoxypyridin-3-yl)methyl)amino)cyclobutan-1-ol;
(1s,3s)-3-(((6-(2,2′-dichloro-3′-(2-((((1s,3s)-3-hydroxycyclobutyl)amino)methyl)-1-methyl-1H-imidazo[4,5-b]pyridin-5-yl)-[1,1′-biphenyl]-3-yl)-2-methoxypyridin-3-yl)methyl)amino)cyclobutan-1-ol;
(S)-5-((((6-(2,2′-dichloro-3′-(3-methyl-2-(((((S)-5-oxopyrrolidin-2-yl)methyl)amino)methyl)-3H-imidazo[4,5-b]pyridin-5-yl)-[1,1′-biphenyl]-3-yl)-2-methoxypyridin-3-yl)methyl)amino)methyl)pyrrolidin-2-one;
(R)-5-((((6-(2,2′-dichloro-3′-(3-methyl-2-(((((S)-5-oxopyrrolidin-2-yl)methyl)amino)methyl)-3H-imidazo[4,5-b]pyridin-5-yl)-[1,1′-biphenyl]-3-yl)-2-methoxypyridin-3-yl)methyl)amino)methyl)pyrrolidin-2-one;
(S)-5-((((6-(2,2′-dichloro-3′-(3-methyl-2-(((((R)-5-oxopyrrolidin-2-yl)methyl)amino)methyl)-3H-imidazo[4,5-b]pyridin-5-yl)-[1,1′-biphenyl]-3-yl)-2-methoxypyridin-3-yl)methyl)amino)methyl)pyrrolidin-2-one;
(R)-5-((((6-(2,2′-dichloro-3′-(3-methyl-2-(((((R)-5-oxopyrrolidin-2-yl)methyl)amino)methyl)-3H-imidazo[4,5-b]pyridin-5-yl)-[1,1′-biphenyl]-3-yl)-2-methoxypyridin-3-yl)methyl)amino)methyl)pyrrolidin-2-one;
(1r,3r)-3-(((6-(2,2′-dichloro-3′-(2-((((1r,3r)-3-hydroxycyclobutyl)amino)methyl)-3-methyl-3H-imidazo[4,5-b]pyridin-5-yl)-[1,1′-biphenyl]-3-yl)-2-methoxypyridin-3-yl)methyl)amino)cyclobutan-1-ol;
(1s,3s)-3-(((6-(2,2′-dichloro-3′-(2-((((1r,3r)-3-hydroxycyclobutyl)amino)methyl)-3-methyl-3H-imidazo[4,5-b]pyridin-5-yl)-[1,1′-biphenyl]-3-yl)-2-methoxypyridin-3-yl)methyl)amino)cyclobutan-1-ol;
(1r,3r)-3-(((6-(2,2′-dichloro-3′-(2-((((1s,3s)-3-hydroxycyclobutyl)amino)methyl)-3-methyl-3H-imidazo[4,5-b]pyridin-5-yl)-[1,1′-biphenyl]-3-yl)-2-methoxypyridin-3-yl)methyl)amino)cyclobutan-1-ol;
(1s,3s)-3-(((6-(2,2′-dichloro-3′-(2-((((1s,3s)-3-hydroxycyclobutyl)amino)methyl)-3-methyl-3H-imidazo[4,5-b]pyridin-5-yl)-[1,1′-biphenyl]-3-yl)-2-methoxypyridin-3-yl)methyl)amino)cyclobutan-1-ol;
(S)-8-(2,2′-dichloro-3′-(6-methoxy-5-((((5-oxopyrrolidin-2-yl)methyl)amino)methyl)pyridin-2-yl)-[1,1′-biphenyl]-3-yl)-3-(((3-hydroxypropyl)amino)methyl)-4H-pyrido[1,2-a]pyrimidin-4-one;
(R)-8-(2,2′-dichloro-3′-(6-methoxy-5-((((5-oxopyrrolidin-2-yl)methyl)amino)methyl)pyridin-2-yl)-[1,1′-biphenyl]-3-yl)-3-(((3-hydroxypropyl)amino)methyl)-4H-pyrido[1,2-a]pyrimidin-4-one;
(S)-8-(2,2′-dichloro-3′-(6-methoxy-5-((((5-oxopyrrolidin-2-yl)methyl)amino)methyl)pyridin-2-yl)-[1,1′-biphenyl]-3-yl)-3-(((3-hydroxypropyl)(methyl)amino)methyl)-4H-pyrido[1,2-a]pyrimidin-4-one;
(R)-8-(2,2′-dichloro-3′-(6-methoxy-5-((((5-oxopyrrolidin-2-yl)methyl)amino)methyl)pyridin-2-yl)-[1,1′-biphenyl]-3-yl)-3-(((3-hydroxypropyl)(methyl)amino)methyl)-4H-pyrido[1,2-a]pyrimidin-4-one;
8-(2,2′-dichloro-3′-(6-methoxy-5-(((((S)-5-oxopyrrolidin-2-yl)methyl)amino)methyl)pyridin-2-yl)-[1,1′-biphenyl]-3-yl)-3-((((1r,3r)-3-hydroxy-3-methylcyclobutyl)(methyl)amino)methyl)-4H-pyrido[1,2-a]pyrimidin-4-one;
8-(2,2′-dichloro-3′-(6-methoxy-5-(((((S)-5-oxopyrrolidin-2-yl)methyl)amino)methyl)pyridin-2-yl)-[1,1′-biphenyl]-3-yl)-3-((((1s,3s)-3-hydroxy-3-methylcyclobutyl)(methyl)amino)methyl)-4H-pyrido[1,2-a]pyrimidin-4-one;
8-(2,2′-dichloro-3′-(6-methoxy-5-(((((R)-5-oxopyrrolidin-2-yl)methyl)amino)methyl)pyridin-2-yl)-[1,1′-biphenyl]-3-yl)-3-((((1r,3r)-3-hydroxy-3-methylcyclobutyl)(methyl)amino)methyl)-4H-pyrido[1,2-a]pyrimidin-4-one;
8-(2,2′-dichloro-3′-(6-methoxy-5-(((((R)-5-oxopyrrolidin-2-yl)methyl)amino)methyl)pyridin-2-yl)-[1,1′-biphenyl]-3-yl)-3-((((1s,3s)-3-hydroxy-3-methylcyclobutyl)(methyl)amino)methyl)-4H-pyrido[1,2-a]pyrimidin-4-one;
8-(2,2′-dichloro-3′-(6-methoxy-5-(((((S)-5-oxopyrrolidin-2-yl)methyl)amino)methyl)pyridin-2-yl)-[1,1′-biphenyl]-3-yl)-3-((((S)-2-hydroxypropyl)(methyl)amino)methyl)-4H-pyrido[1,2-a]pyrimidin-4-one;
8-(2,2′-dichloro-3′-(6-methoxy-5-(((((R)-5-oxopyrrolidin-2-yl)methyl)amino)methyl)pyridin-2-yl)-[1,1′-biphenyl]-3-yl)-3-((((S)-2-hydroxypropyl)(methyl)amino)methyl)-4H-pyrido[1,2-a]pyrimidin-4-one;
8-(2,2′-dichloro-3′-(6-methoxy-5-(((((S)-5-oxopyrrolidin-2-yl)methyl)amino)methyl)pyridin-2-yl)-[1,1′-biphenyl]-3-yl)-3-((((R)-2-hydroxypropyl)(methyl)amino)methyl)-4H-pyrido[1,2-a]pyrimidin-4-one;
8-(2,2′-dichloro-3′-(6-methoxy-5-(((((R)-5-oxopyrrolidin-2-yl)methyl)amino)methyl)pyridin-2-yl)-[1,1′-biphenyl]-3-yl)-3-((((R)-2-hydroxypropyl)(methyl)amino)methyl)-4H-pyrido[1,2-a]pyrimidin-4-one;
ethyl (S)-4-(((8-(2,2′-dichloro-3′-(6-methoxy-5-(((((S)-5-oxopyrrolidin-2-yl)methyl)amino)methyl)pyridin-2-yl)-[1,1′-biphenyl]-3-yl)-4-oxo-4H-pyrido[1,2-a]pyrimidin-3-yl)methyl)amino)-3-hydroxybutanoate;
ethyl (R)-4-(((8-(2,2′-dichloro-3′-(6-methoxy-5-(((((S)-5-oxopyrrolidin-2-yl)methyl)amino)methyl)pyridin-2-yl)-[1,1′-biphenyl]-3-yl)-4-oxo-4H-pyrido[1,2-a]pyrimidin-3-yl)methyl)amino)-3-hydroxybutanoate;
ethyl (S)-4-(((8-(2,2′-dichloro-3′-(6-methoxy-5-(((((R)-5-oxopyrrolidin-2-yl)methyl)amino)methyl)pyridin-2-yl)-[1,1′-biphenyl]-3-yl)-4-oxo-4H-pyrido[1,2-a]pyrimidin-3-yl)methyl)amino)-3-hydroxybutanoate;
ethyl (R)-4-(((8-(2,2′-dichloro-3′-(6-methoxy-5-(((((R)-5-oxopyrrolidin-2-yl)methyl)amino)methyl)pyridin-2-yl)-[1,1′-biphenyl]-3-yl)-4-oxo-4H-pyrido[1,2-a]pyrimidin-3-yl)methyl)amino)-3-hydroxybutanoate;
8-(2,2′-dichloro-3′-(6-methoxy-5-(((2-(2-oxopyrrolidin-1-yl)ethyl)amino)methyl)pyridin-2-yl)-[1,1′-biphenyl]-3-yl)-3-(((2-(2-oxopyrrolidin-1-yl)ethyl)amino)methyl)-4H-pyrido[1,2-a]pyrimidin-4-one;
3-(((1-acetylpiperidin-4-yl)amino)methyl)-8-(3′-(5-(((1-acetylpiperidin-4-yl)amino)methyl)-6-methoxypyridin-2-yl)-2,2′-dichloro-[1,1′-biphenyl]-3-yl)-4H-pyrido[1,2-a]pyrimidin-4-one;
8-(2,2′-dichloro-3′-(6-methoxy-5-(((((S)-5-oxopyrrolidin-2-yl)methyl)amino)methyl)pyridin-2-yl)-[1,1′-biphenyl]-3-yl)-3-(5-hydroxy-1,4,5,6-tetrahydropyrimidin-2-yl)-6-methyl-4H-pyrido[1,2-a]pyrimidin-4-one;
8-(2,2′-dichloro-3′-(6-methoxy-5-(((((R)-5-oxopyrrolidin-2-yl)methyl)amino)methyl)pyridin-2-yl)-[1,1′-biphenyl]-3-yl)-3-(5-hydroxy-1,4,5,6-tetrahydropyrimidin-2-yl)-6-methyl-4H-pyrido[1,2-a]pyrimidin-4-one;
7-(2,2′-dichloro-3′-(6-methoxy-5-(((((S)-5-oxopyrrolidin-2-yl)methyl)amino)methyl)pyridin-2-yl)-[1,1′-biphenyl]-3-yl)-2-((((S)-2-hydroxypropyl)amino)methyl)-4H-pyrido[1,2-a]pyrimidin-4-one;
7-(2,2′-dichloro-3′-(6-methoxy-5-(((((R)-5-oxopyrrolidin-2-yl)methyl)amino)methyl)pyridin-2-yl)-[1,1′-biphenyl]-3-yl)-2-((((S)-2-hydroxypropyl)amino)methyl)-4H-pyrido[1,2-a]pyrimidin-4-one;
7-(2,2′-dichloro-3′-(6-methoxy-5-(((((S)-5-oxopyrrolidin-2-yl)methyl)amino)methyl)pyridin-2-yl)-[1,1′-biphenyl]-3-yl)-2-((((R)-2-hydroxypropyl)amino)methyl)-4H-pyrido[1,2-a]pyrimidin-4-one;
7-(2,2′-dichloro-3′-(6-methoxy-5-(((((R)-5-oxopyrrolidin-2-yl)methyl)amino)methyl)pyridin-2-yl)-[1,1′-biphenyl]-3-yl)-2-((((R)-2-hydroxypropyl)amino)methyl)-4H-pyrido[1,2-a]pyrimidin-4-one;
7-(2,2′-dichloro-3′-(6-methoxy-5-(((((S)-5-oxopyrrolidin-2-yl)methyl)amino)methyl)pyridin-2-yl)-[1,1′-biphenyl]-3-yl)-2-((((1r,3r)-3-hydroxy-3-methylcyclobutyl)amino)methyl)-4H-pyrido[1,2-a]pyrimidin-4-one;
7-(2,2′-dichloro-3′-(6-methoxy-5-(((((S)-5-oxopyrrolidin-2-yl)methyl)amino)methyl)pyridin-2-yl)-[1,1′-biphenyl]-3-yl)-2-((((1s,3s)-3-hydroxy-3-methylcyclobutyl)amino)methyl)-4H-pyrido[1,2-a]pyrimidin-4-one;
7-(2,2′-dichloro-3′-(6-methoxy-5-(((((R)-5-oxopyrrolidin-2-yl)methyl)amino)methyl)pyridin-2-yl)-[1,1′-biphenyl]-3-yl)-2-((((1r,3r)-3-hydroxy-3-methylcyclobutyl)amino)methyl)-4H-pyrido[1,2-a]pyrimidin-4-one;
7-(2,2′-dichloro-3′-(6-methoxy-5-(((((R)-5-oxopyrrolidin-2-yl)methyl)amino)methyl)pyridin-2-yl)-[1,1′-biphenyl]-3-yl)-2-((((1s,3s)-3-hydroxy-3-methylcyclobutyl)amino)methyl)-4H-pyrido[1,2-a]pyrimidin-4-one;
(S)-5-((((6-(2,2′-dichloro-3′-((S)-4-((((S)-5-oxopyrrolidin-2-yl)methyl)amino)chroman-7-yl)-[1,1′-biphenyl]-3-yl)-2-methoxypyridin-3-yl)methyl)amino)methyl)pyrrolidin-2-one;
(S)-5-((((6-(2,2′-dichloro-3′-((R)-4-((((S)-5-oxopyrrolidin-2-yl)methyl)amino)chroman-7-yl)-[1,1′-biphenyl]-3-yl)-2-methoxypyridin-3-yl)methyl)amino)methyl)pyrrolidin-2-one;
(S)-5-((((6-(2,2′-dichloro-3′-((S)-4-((((R)-5-oxopyrrolidin-2-yl)methyl)amino)chroman-7-yl)-[1,1′-biphenyl]-3-yl)-2-methoxypyridin-3-yl)methyl)amino)methyl)pyrrolidin-2-one;
(S)-5-((((6-(2,2′-dichloro-3′-((R)-4-((((R)-5-oxopyrrolidin-2-yl)methyl)amino)chroman-7-yl)-[1,1′-biphenyl]-3-yl)-2-methoxypyridin-3-yl)methyl)amino)methyl)pyrrolidin-2-one;
(R)-5-((((6-(2,2′-dichloro-3′-((S)-4-((((S)-5-oxopyrrolidin-2-yl)methyl)amino)chroman-7-yl)-[1,1′-biphenyl]-3-yl)-2-methoxypyridin-3-yl)methyl)amino)methyl)pyrrolidin-2-one;
(R)-5-((((6-(2,2′-dichloro-3′-((R)-4-((((S)-5-oxopyrrolidin-2-yl)methyl)amino)chroman-7-yl)-[1,1′-biphenyl]-3-yl)-2-methoxypyridin-3-yl)methyl)amino)methyl)pyrrolidin-2-one;
(R)-5-((((6-(2,2′-dichloro-3′-((S)-4-((((R)-5-oxopyrrolidin-2-yl)methyl)amino)chroman-7-yl)-[1,1′-biphenyl]-3-yl)-2-methoxypyridin-3-yl)methyl)amino)methyl)pyrrolidin-2-one;
(R)-5-((((6-(2,2′-dichloro-3′-((R)-4-((((R)-5-oxopyrrolidin-2-yl)methyl)amino)chroman-7-yl)-[1,1′-biphenyl]-3-yl)-2-methoxypyridin-3-yl)methyl)amino)methyl)pyrrolidin-2-one;
(S)-8-(2,2′-dichloro-3′-(6-methoxy-5-((((5-oxopyrrolidin-2-yl)methyl)amino)methyl)pyridin-2-yl)-[1,1′-biphenyl]-3-yl)-3-((5-oxo-1,4-diazepan-1-yl)methyl)-4H-pyrido[1,2-a]pyrimidin-4-one;
(R)-8-(2,2′-dichloro-3′-(6-methoxy-5-((((5-oxopyrrolidin-2-yl)methyl)amino)methyl)pyridin-2-yl)-[1,1′-biphenyl]-3-yl)-3-((5-oxo-1,4-diazepan-1-yl)methyl)-4H-pyrido[1,2-a]pyrimidin-4-one;
(S)-3-((3,3-bis(hydroxymethyl)pyrrolidin-1-yl)methyl)-8-(2,2′-dichloro-3′-(6-methoxy-5-((((5-oxopyrrolidin-2-yl)methyl)amino)methyl)pyridin-2-yl)-[1,1′-biphenyl]-3-yl)-4H-pyrido[1,2-a]pyrimidin-4-one;
(R)-3-((3,3-bis(hydroxymethyl)pyrrolidin-1-yl)methyl)-8-(2,2′-dichloro-3′-(6-methoxy-5-((((5-oxopyrrolidin-2-yl)methyl)amino)methyl)pyridin-2-yl)-[1,1′-biphenyl]-3-yl)-4H-pyrido[1,2-a]pyrimidin-4-one;
(S)-3-((6-acetyl-2,6-diazaspiro[3.3]heptan-2-yl)methyl)-8-(2,2′-dichloro-3′-(6-methoxy-5-((((5-oxopyrrolidin-2-yl)methyl)amino)methyl)pyridin-2-yl)-[1,1′-biphenyl]-3-yl)-4H-pyrido[1,2-a]pyrimidin-4-one;
(R)-3-((6-acetyl-2,6-diazaspiro[3.3]heptan-2-yl)methyl)-8-(2,2′-dichloro-3′-(6-methoxy-5-((((5-oxopyrrolidin-2-yl)methyl)amino)methyl)pyridin-2-yl)-[1,1′-biphenyl]-3-yl)-4H-pyrido[1,2-a]pyrimidin-4-one;
N-((6-(2,2′-dichloro-3′-(6-methoxy-5-(((((S)-5-oxopyrrolidin-2-yl)methyl)amino)methyl)pyridin-2-yl)-[1,1′-biphenyl]-3-yl)-2-methoxypyridin-3-yl)methyl)-N—(((S)-5-oxopyrrolidin-2-yl)methyl)formamide;
N-((6-(2,2′-dichloro-3′-(6-methoxy-5-(((((R)-5-oxopyrrolidin-2-yl)methyl)amino)methyl)pyridin-2-yl)-[1,1′-biphenyl]-3-yl)-2-methoxypyridin-3-yl)methyl)-N—(((S)-5-oxopyrrolidin-2-yl)methyl)formamide;
N-((6-(2,2′-dichloro-3′-(6-methoxy-5-(((((S)-5-oxopyrrolidin-2-yl)methyl)amino)methyl)pyridin-2-yl)-[1,1′-biphenyl]-3-yl)-2-methoxypyridin-3-yl)methyl)-N—(((R)-5-oxopyrrolidin-2-yl)methyl)formamide;
N-((6-(2,2′-dichloro-3′-(6-methoxy-5-(((((R)-5-oxopyrrolidin-2-yl)methyl)amino)methyl)pyridin-2-yl)-[1,1′-biphenyl]-3-yl)-2-methoxypyridin-3-yl)methyl)-N—(((R)-5-oxopyrrolidin-2-yl)methyl)formamide;
6-(2,2′-dichloro-3′-(6-methoxy-5-(((((S)-5-oxopyrrolidin-2-yl)methyl)amino)methyl)pyridin-2-yl)-[1,1′-biphenyl]-3-yl)-3-methyl-2-(((((S)-5-oxopyrrolidin-2-yl)methyl)amino)methyl)pyrrolo[2,1-f][1,2,4]triazin-4(3H)-one;
6-(2,2′-dichloro-3′-(6-methoxy-5-(((((R)-5-oxopyrrolidin-2-yl)methyl)amino)methyl)pyridin-2-yl)-[1,1′-biphenyl]-3-yl)-3-methyl-2-(((((S)-5-oxopyrrolidin-2-yl)methyl)amino)methyl)pyrrolo[2,1-f][1,2,4]triazin-4(3H)-one;
6-(2,2′-dichloro-3′-(6-methoxy-5-(((((S)-5-oxopyrrolidin-2-yl)methyl)amino)methyl)pyridin-2-yl)-[1,1′-biphenyl]-3-yl)-3-methyl-2-(((((R)-5-oxopyrrolidin-2-yl)methyl)amino)methyl)pyrrolo[2,1-f][1,2,4]triazin-4(3H)-one;
6-(2,2′-dichloro-3′-(6-methoxy-5-(((((R)-5-oxopyrrolidin-2-yl)methyl)amino)methyl)pyridin-2-yl)-[1,1′-biphenyl]-3-yl)-3-methyl-2-(((((R)-5-oxopyrrolidin-2-yl)methyl)amino)methyl)pyrrolo[2,1-f][1,2,4]triazin-4(3H)-one;
6-(2,2′-dichloro-3′-(6-methoxy-5-(((((S)-5-oxopyrrolidin-2-yl)methyl)amino)methyl)pyridin-2-yl)-[1,1′-biphenyl]-3-yl)-2-((((1r,3r)-3-hydroxy-3-methylcyclobutyl)amino)methyl)-3-methylpyrrolo[2,1-f][1,2,4]triazin-4(3H)-one;
6-(2,2′-dichloro-3′-(6-methoxy-5-(((((S)-5-oxopyrrolidin-2-yl)methyl)amino)methyl)pyridin-2-yl)-[1,1′-biphenyl]-3-yl)-2-((((1s,3s)-3-hydroxy-3-methylcyclobutyl)amino)methyl)-3-methylpyrrolo[2,1-f][1,2,4]triazin-4(3H)-one;
6-(2,2′-dichloro-3′-(6-methoxy-5-(((((R)-5-oxopyrrolidin-2-yl)methyl)amino)methyl)pyridin-2-yl)-[1,1′-biphenyl]-3-yl)-2-((((1r,3r)-3-hydroxy-3-methylcyclobutyl)amino)methyl)-3-methylpyrrolo[2,1-f][1,2,4]triazin-4(3H)-one;
6-(2,2′-dichloro-3′-(6-methoxy-5-(((((R)-5-oxopyrrolidin-2-yl)methyl)amino)methyl)pyridin-2-yl)-[1,1′-biphenyl]-3-yl)-2-((((1s,3s)-3-hydroxy-3-methylcyclobutyl)amino)methyl)-3-methylpyrrolo[2,1-f][1,2,4]triazin-4(3H)-one;
(S)-5-((((6-(2,2′-dichloro-3′-(3-((((S)-2-hydroxypropyl)amino)methyl)-1,6-naphthyridin-7-yl)-[1,1′-biphenyl]-3-yl)-2-methoxypyridin-3-yl)methyl)amino)methyl)pyrrolidin-2-one;
(S)-5-((((6-(2,2′-dichloro-3′-(3-((((R)-2-hydroxypropyl)amino)methyl)-1,6-naphthyridin-7-yl)-[1,1′-biphenyl]-3-yl)-2-methoxypyridin-3-yl)methyl)amino)methyl)pyrrolidin-2-one;
(R)-5-((((6-(2,2′-dichloro-3′-(3-((((S)-2-hydroxypropyl)amino)methyl)-1,6-naphthyridin-7-yl)-[1,1′-biphenyl]-3-yl)-2-methoxypyridin-3-yl)methyl)amino)methyl)pyrrolidin-2-one;
(R)-5-((((6-(2,2′-dichloro-3′-(3-((((R)-2-hydroxypropyl)amino)methyl)-1,6-naphthyridin-7-yl)-[1,1′-biphenyl]-3-yl)-2-methoxypyridin-3-yl)methyl)amino)methyl)pyrrolidin-2-one;
(S)-5-((((6-(2,2′-dichloro-3′-(3-(((2-hydroxyethyl)amino)methyl)-1,6-naphthyridin-7-yl)-[1,1′-biphenyl]-3-yl)-2-methoxypyridin-3-yl)methyl)amino)methyl)pyrrolidin-2-one;
(R)-5-((((6-(2,2′-dichloro-3′-(3-(((2-hydroxyethyl)amino)methyl)-1,6-naphthyridin-7-yl)-[1,1′-biphenyl]-3-yl)-2-methoxypyridin-3-yl)methyl)amino)methyl)pyrrolidin-2-one;
8-(2,2′-dichloro-3′-(4-chloro-6-methoxy-5-(((((S)-5-oxopyrrolidin-2-yl)methyl)amino)methyl)pyridin-2-yl)-[1,1′-biphenyl]-3-yl)-3-(((((S)-5-oxopyrrolidin-2-yl)methyl)amino)methyl)-4H-pyrido[1,2-a]pyrimidin-4-one;
8-(2,2′-dichloro-3′-(4-chloro-6-methoxy-5-(((((R)-5-oxopyrrolidin-2-yl)methyl)amino)methyl)pyridin-2-yl)-[1,1′-biphenyl]-3-yl)-3-(((((S)-5-oxopyrrolidin-2-yl)methyl)amino)methyl)-4H-pyrido[1,2-a]pyrimidin-4-one;
8-(2,2′-dichloro-3′-(4-chloro-6-methoxy-5-(((((S)-5-oxopyrrolidin-2-yl)methyl)amino)methyl)pyridin-2-yl)-[1,1′-biphenyl]-3-yl)-3-(((((R)-5-oxopyrrolidin-2-yl)methyl)amino)methyl)-4H-pyrido[1,2-a]pyrimidin-4-one;
8-(2,2′-dichloro-3′-(4-chloro-6-methoxy-5-(((((R)-5-oxopyrrolidin-2-yl)methyl)amino)methyl)pyridin-2-yl)-[1,1′-biphenyl]-3-yl)-3-(((((R)-5-oxopyrrolidin-2-yl)methyl)amino)methyl)-4H-pyrido[1,2-a]pyrimidin-4-one;
(S)-4-(((8-(2,2′-dichloro-3′-(4-fluoro-6-methoxy-5-(((((S)-5-oxopyrrolidin-2-yl)methyl)amino)methyl)pyridin-2-yl)-[1,1′-biphenyl]-3-yl)-4-oxo-4H-pyrido[1,2-a]pyrimidin-3-yl)methyl)amino)-3-hydroxy-N,N-dimethylbutanamide;
(R)-4-(((8-(2,2′-dichloro-3′-(4-fluoro-6-methoxy-5-(((((S)-5-oxopyrrolidin-2-yl)methyl)amino)methyl)pyridin-2-yl)-[1,1′-biphenyl]-3-yl)-4-oxo-4H-pyrido[1,2-a]pyrimidin-3-yl)methyl)amino)-3-hydroxy-N,N-dimethylbutanamide;
(S)-4-(((8-(2,2′-dichloro-3′-(4-fluoro-6-methoxy-5-(((((R)-5-oxopyrrolidin-2-yl)methyl)amino)methyl)pyridin-2-yl)-[1,1′-biphenyl]-3-yl)-4-oxo-4H-pyrido[1,2-a]pyrimidin-3-yl)methyl)amino)-3-hydroxy-N,N-dimethylbutanamide;
(R)-4-(((8-(2,2′-dichloro-3′-(4-fluoro-6-methoxy-5-(((((R)-5-oxopyrrolidin-2-yl)methyl)amino)methyl)pyridin-2-yl)-[1,1′-biphenyl]-3-yl)-4-oxo-4H-pyrido[1,2-a]pyrimidin-3-yl)methyl)amino)-3-hydroxy-N,N-dimethylbutanamide;
8-(2,2′-dichloro-3′-(4-chloro-6-methoxy-3-(((((R)-5-oxopyrrolidin-2-yl)methyl)amino)methyl)pyridin-2-yl)-[1,1′-biphenyl]-3-yl)-3-(((((S)-5-oxopyrrolidin-2-yl)methyl)amino)methyl)-4H-pyrido[1,2-a]pyrimidin-4-one;
8-(2,2′-dichloro-3′-(4-chloro-6-methoxy-3-(((((R)-5-oxopyrrolidin-2-yl)methyl)amino)methyl)pyridin-2-yl)-[1,1′-biphenyl]-3-yl)-3-(((((R)-5-oxopyrrolidin-2-yl)methyl)amino)methyl)-4H-pyrido[1,2-a]pyrimidin-4-one;
8-(2,2′-dichloro-3′-(4-chloro-6-methoxy-3-(((((S)-5-oxopyrrolidin-2-yl)methyl)amino)methyl)pyridin-2-yl)-[1,1′-biphenyl]-3-yl)-3-(((((S)-5-oxopyrrolidin-2-yl)methyl)amino)methyl)-4H-pyrido[1,2-a]pyrimidin-4-one;
8-(2,2′-dichloro-3′-(4-chloro-6-methoxy-3-(((((S)-5-oxopyrrolidin-2-yl)methyl)amino)methyl)pyridin-2-yl)-[1,1′-biphenyl]-3-yl)-3-(((((R)-5-oxopyrrolidin-2-yl)methyl)amino)methyl)-4H-pyrido[1,2-a]pyrimidin-4-one;
(S)-5-((((6-(2,2′-dichloro-3′-(2-(((((S)-5-oxopyrrolidin-2-yl)methyl)amino)methyl)imidazo[1,2-b]pyridazin-6-yl)-[1,1′-biphenyl]-3-yl)-2-methoxypyridin-3-yl)methyl)amino)methyl)pyrrolidin-2-one;
(R)-5-((((6-(2,2′-dichloro-3′-(2-(((((S)-5-oxopyrrolidin-2-yl)methyl)amino)methyl)imidazo[1,2-b]pyridazin-6-yl)-[1,1′-biphenyl]-3-yl)-2-methoxypyridin-3-yl)methyl)amino)methyl)pyrrolidin-2-one;
(S)-5-((((6-(2,2′-dichloro-3′-(2-(((((R)-5-oxopyrrolidin-2-yl)methyl)amino)methyl)imidazo[1,2-b]pyridazin-6-yl)-[1,1′-biphenyl]-3-yl)-2-methoxypyridin-3-yl)methyl)amino)methyl)pyrrolidin-2-one;
(R)-5-((((6-(2,2′-dichloro-3′-(2-(((((R)-5-oxopyrrolidin-2-yl)methyl)amino)methyl)imidazo[1,2-b]pyridazin-6-yl)-[1,1′-biphenyl]-3-yl)-2-methoxypyridin-3-yl)methyl)amino)methyl)pyrrolidin-2-one;
2-(((6-(2,2′-dichloro-3′-(2-(((2-hydroxyethyl)(methyl)amino)methyl)imidazo[1,2-b]pyridazin-6-yl)-[1,1′-biphenyl]-3-yl)-2-methoxypyridin-3-yl)methyl)(methyl)amino)ethan-1-01;
(S)-1-(((6-(2,2′-dichloro-3′-(2-((((S)-2-hydroxypropyl)amino)methyl)imidazo[1,2-b]pyridazin-6-yl)-[1,1′-biphenyl]-3-yl)-2-methoxypyridin-3-yl)methyl)amino)propan-2-ol;
(R)-1-(((6-(2,2′-dichloro-3′-(2-((((S)-2-hydroxypropyl)amino)methyl)imidazo[1,2-b]pyridazin-6-yl)-[1,1′-biphenyl]-3-yl)-2-methoxypyridin-3-yl)methyl)amino)propan-2-ol;
(S)-1-(((6-(2,2′-dichloro-3′-(2-((((R)-2-hydroxypropyl)amino)methyl)imidazo[1,2-b]pyridazin-6-yl)-[1,1′-biphenyl]-3-yl)-2-methoxypyridin-3-yl)methyl)amino)propan-2-ol;
(R)-1-(((6-(2,2′-dichloro-3′-(2-((((R)-2-hydroxypropyl)amino)methyl)imidazo[1,2-b]pyridazin-6-yl)-[1,1′-biphenyl]-3-yl)-2-methoxypyridin-3-yl)methyl)amino)propan-2-ol;
(S)-2-(((8-(2,2′-dichloro-3′-(6-methoxy-5-((((5-oxopyrrolidin-2-yl)methyl)amino)methyl)pyridin-2-yl)-[1,1′-biphenyl]-3-yl)-4-oxo-4H-pyrido[1,2-a]pyrimidin-2-yl)methyl)amino)-N,2-dimethylpropanamide;
(R)-2-(((8-(2,2′-dichloro-3′-(6-methoxy-5-((((5-oxopyrrolidin-2-yl)methyl)amino)methyl)pyridin-2-yl)-[1,1′-biphenyl]-3-yl)-4-oxo-4H-pyrido[1,2-a]pyrimidin-2-yl)methyl)amino)-N,2-dimethylpropanamide;
8-(2,2′-dichloro-3′-(6-methoxy-5-(((((S)-5-oxopyrrolidin-2-yl)methyl)amino)methyl)pyridin-2-yl)-[1,1′-biphenyl]-3-yl)-2-(((S)-4-hydroxy-2-oxopyrrolidin-1-yl)methyl)-4H-pyrido[1,2-a]pyrimidin-4-one;
8-(2,2′-dichloro-3′-(6-methoxy-5-(((((R)-5-oxopyrrolidin-2-yl)methyl)amino)methyl)pyridin-2-yl)-[1,1′-biphenyl]-3-yl)-2-(((S)-4-hydroxy-2-oxopyrrolidin-1-yl)methyl)-4H-pyrido[1,2-a]pyrimidin-4-one;
8-(2,2′-dichloro-3′-(6-methoxy-5-(((((S)-5-oxopyrrolidin-2-yl)methyl)amino)methyl)pyridin-2-yl)-[1,1′-biphenyl]-3-yl)-2-(((R)-4-hydroxy-2-oxopyrrolidin-1-yl)methyl)-4H-pyrido[1,2-a]pyrimidin-4-one;
8-(2,2′-dichloro-3′-(6-methoxy-5-(((((R)-5-oxopyrrolidin-2-yl)methyl)amino)methyl)pyridin-2-yl)-[1,1′-biphenyl]-3-yl)-2-(((R)-4-hydroxy-2-oxopyrrolidin-1-yl)methyl)-4H-pyrido[1,2-a]pyrimidin-4-one;
methyl (S)-1-((8-(2,2′-dichloro-3′-(6-methoxy-5-((((5-oxopyrrolidin-2-yl)methyl)amino)methyl)pyridin-2-yl)-[1,1′-biphenyl]-3-yl)-4-oxo-4H-pyrido[1,2-a]pyrimidin-2-yl)methyl)azetidine-3-carboxylate;
methyl (R)-1-((8-(2,2′-dichloro-3′-(6-methoxy-5-((((5-oxopyrrolidin-2-yl)methyl)amino)methyl)pyridin-2-yl)-[1,1′-biphenyl]-3-yl)-4-oxo-4H-pyrido[1,2-a]pyrimidin-2-yl)methyl)azetidine-3-carboxylate;
(S)-5-((((6-(2,2′-dichloro-3′-(2,3,4,5-tetrahydro-1H-benzo[c]azepin-7-yl)-[1,1′-biphenyl]-3-yl)-2-methoxypyridin-3-yl)methyl)amino)methyl)pyrrolidin-2-one;
(R)-5-((((6-(2,2′-dichloro-3′-(2,3,4,5-tetrahydro-1H-benzo[c]azepin-7-yl)-[1,1′-biphenyl]-3-yl)-2-methoxypyridin-3-yl)methyl)amino)methyl)pyrrolidin-2-one;
(S)-8-(2,2′-dichloro-3′-(6-methoxy-5-((((5-oxopyrrolidin-2-yl)methyl)amino)methyl)pyridin-2-yl)-[1,1′-biphenyl]-3-yl)-3-(((2-methoxy-2-methylpropyl)amino)methyl)-4H-pyrido[1,2-a]pyrimidin-4-one;
(R)-8-(2,2′-dichloro-3′-(6-methoxy-5-((((5-oxopyrrolidin-2-yl)methyl)amino)methyl)pyridin-2-yl)-[1,1′-biphenyl]-3-yl)-3-(((2-methoxy-2-methylpropyl)amino)methyl)-4H-pyrido[1,2-a]pyrimidin-4-one;
(S)-8-(2,2′-dichloro-3′-(6-methoxy-5-((((5-oxopyrrolidin-2-yl)methyl)amino)methyl)pyridin-2-yl)-[1,1′-biphenyl]-3-yl)-3-((6-hydroxy-2-azaspiro[3.3]heptan-2-yl)methyl)-4H-pyrido[1,2-a]pyrimidin-4-one;
(R)-8-(2,2′-dichloro-3′-(6-methoxy-5-((((5-oxopyrrolidin-2-yl)methyl)amino)methyl)pyridin-2-yl)-[1,1′-biphenyl]-3-yl)-3-((6-hydroxy-2-azaspiro[3.3]heptan-2-yl)methyl)-4H-pyrido[1,2-a]pyrimidin-4-one;
8-(2,2′-dichloro-3′-(6-methoxy-5-(((((S)-5-oxopyrrolidin-2-yl)methyl)amino)methyl)pyridin-2-yl)-[1,1′-biphenyl]-3-yl)-3-(((3R,4R)-3,4-dihydroxypyrrolidin-1-yl)methyl)-4H-pyrido[1,2-a]pyrimidin-4-one;
8-(2,2′-dichloro-3′-(6-methoxy-5-(((((S)-5-oxopyrrolidin-2-yl)methyl)amino)methyl)pyridin-2-yl)-[1,1′-biphenyl]-3-yl)-3-(((3R,4S)-3,4-dihydroxypyrrolidin-1-yl)methyl)-4H-pyrido[1,2-a]pyrimidin-4-one;
8-(2,2′-dichloro-3′-(6-methoxy-5-(((((S)-5-oxopyrrolidin-2-yl)methyl)amino)methyl)pyridin-2-yl)-[1,1′-biphenyl]-3-yl)-3-(((3S,4R)-3,4-dihydroxypyrrolidin-1-yl)methyl)-4H-pyrido[1,2-a]pyrimidin-4-one;
8-(2,2′-dichloro-3′-(6-methoxy-5-(((((S)-5-oxopyrrolidin-2-yl)methyl)amino)methyl)pyridin-2-yl)-[1,1′-biphenyl]-3-yl)-3-(((3S,4S)-3,4-dihydroxypyrrolidin-1-yl)methyl)-4H-pyrido[1,2-a]pyrimidin-4-one;
8-(2,2′-dichloro-3′-(6-methoxy-5-(((((R)-5-oxopyrrolidin-2-yl)methyl)amino)methyl)pyridin-2-yl)-[1,1′-biphenyl]-3-yl)-3-(((3R,4R)-3,4-dihydroxypyrrolidin-1-yl)methyl)-4H-pyrido[1,2-a]pyrimidin-4-one;
8-(2,2′-dichloro-3′-(6-methoxy-5-(((((R)-5-oxopyrrolidin-2-yl)methyl)amino)methyl)pyridin-2-yl)-[1,1′-biphenyl]-3-yl)-3-(((3R,4S)-3,4-dihydroxypyrrolidin-1-yl)methyl)-4H-pyrido[1,2-a]pyrimidin-4-one;
8-(2,2′-dichloro-3′-(6-methoxy-5-(((((R)-5-oxopyrrolidin-2-yl)methyl)amino)methyl)pyridin-2-yl)-[1,1′-biphenyl]-3-yl)-3-(((3S,4R)-3,4-dihydroxypyrrolidin-1-yl)methyl)-4H-pyrido[1,2-a]pyrimidin-4-one;
8-(2,2′-dichloro-3′-(6-methoxy-5-(((((R)-5-oxopyrrolidin-2-yl)methyl)amino)methyl)pyridin-2-yl)-[1,1′-biphenyl]-3-yl)-3-(((3S,4S)-3,4-dihydroxypyrrolidin-1-yl)methyl)-4H-pyrido[1,2-a]pyrimidin-4-one;
8-(2,2′-dichloro-3′-(6-methoxy-5-(((((R)-5-oxopyrrolidin-2-yl)methyl)amino)methyl)pyridin-2-yl)-[1,1′-biphenyl]-3-yl)-3-((((1r,3r)-3-hydroxy-3-methylcyclobutyl)amino)methyl)-2-methoxy-4H-pyrido[1,2-a]pyrimidin-4-one;
8-(2,2′-dichloro-3′-(6-methoxy-5-(((((S)-5-oxopyrrolidin-2-yl)methyl)amino)methyl)pyridin-2-yl)-[1,1′-biphenyl]-3-yl)-3-((((1s,3s)-3-hydroxy-3-methylcyclobutyl)amino)methyl)-2-methoxy-4H-pyrido[1,2-a]pyrimidin-4-one;
8-(2,2′-dichloro-3′-(6-methoxy-5-(((((R)-5-oxopyrrolidin-2-yl)methyl)amino)methyl)pyridin-2-yl)-[1,1′-biphenyl]-3-yl)-3-((((1s,3s)-3-hydroxy-3-methylcyclobutyl)amino)methyl)-2-methoxy-4H-pyrido[1,2-a]pyrimidin-4-one;
8-(2,2′-dichloro-3′-(6-methoxy-5-(((((S)-5-oxopyrrolidin-2-yl)methyl)amino)methyl)pyridin-2-yl)-[1,1′-biphenyl]-3-yl)-3-((((1r,3r)-3-hydroxy-3-methylcyclobutyl)amino)methyl)-2-methoxy-4H-pyrido[1,2-a]pyrimidin-4-one;
(S)-1-(((8-(2,2′-dichloro-3′-(6-methoxy-5-((((5-oxopyrrolidin-2-yl)methyl)amino)methyl)pyridin-2-yl)-[1,1′-biphenyl]-3-yl)-4-oxo-4H-pyrido[1,2-a]pyrimidin-2-yl)methyl)amino)-N-methylcyclopropane-1-carboxamide;
(R)-1-(((8-(2,2′-dichloro-3′-(6-methoxy-5-((((5-oxopyrrolidin-2-yl)methyl)amino)methyl)pyridin-2-yl)-[1,1′-biphenyl]-3-yl)-4-oxo-4H-pyrido[1,2-a]pyrimidin-2-yl)methyl)amino)-N-methylcyclopropane-1-carboxamide;
6-(2,2′-dichloro-3′-(6-methoxy-5-(((((S)-5-oxopyrrolidin-2-yl)methyl)amino)methyl)pyridin-2-yl)-[1,1′-biphenyl]-3-yl)-2-((((S)-2-hydroxypropyl)amino)methyl)-3-methylpyrrolo[2,1-f][1,2,4]triazin-4(3H)-one;
6-(2,2′-dichloro-3′-(6-methoxy-5-(((((S)-5-oxopyrrolidin-2-yl)methyl)amino)methyl)pyridin-2-yl)-[1,1′-biphenyl]-3-yl)-2-((((R)-2-hydroxypropyl)amino)methyl)-3-methylpyrrolo[2,1-f][1,2,4]triazin-4(3H)-one;
6-(2,2′-dichloro-3′-(6-methoxy-5-(((((R)-5-oxopyrrolidin-2-yl)methyl)amino)methyl)pyridin-2-yl)-[1,1′-biphenyl]-3-yl)-2-((((S)-2-hydroxypropyl)amino)methyl)-3-methylpyrrolo[2,1-f][1,2,4]triazin-4(3H)-one;
6-(2,2′-dichloro-3′-(6-methoxy-5-(((((R)-5-oxopyrrolidin-2-yl)methyl)amino)methyl)pyridin-2-yl)-[1,1′-biphenyl]-3-yl)-2-((((R)-2-hydroxypropyl)amino)methyl)-3-methylpyrrolo[2,1-f][1,2,4]triazin-4(3H)-one;
(S)-6-(2,2′-dichloro-3′-(6-methoxy-5-((((5-oxopyrrolidin-2-yl)methyl)amino)methyl)pyridin-2-yl)-[1,1′-biphenyl]-3-yl)-2-(((2-hydroxyethyl)amino)methyl)-3-methylpyrrolo[2,1-f][1,2,4]triazin-4(3H)-one;
(R)-6-(2,2′-dichloro-3′-(6-methoxy-5-((((5-oxopyrrolidin-2-yl)methyl)amino)methyl)pyridin-2-yl)-[1,1′-biphenyl]-3-yl)-2-(((2-hydroxyethyl)amino)methyl)-3-methylpyrrolo[2,1-f][1,2,4]triazin-4(3H)-one;
(S)-5-((((6-(2,2′-dichloro-3′-(2-(((2-hydroxyethyl)amino)methyl)imidazo[1,2-b]pyridazin-6-yl)-[1,1′-biphenyl]-3-yl)-2-methoxypyridin-3-yl)methyl)amino)methyl)pyrrolidin-2-one;
(R)-5-((((6-(2,2′-dichloro-3′-(2-(((2-hydroxyethyl)amino)methyl)imidazo[1,2-b]pyridazin-6-yl)-[1,1′-biphenyl]-3-yl)-2-methoxypyridin-3-yl)methyl)amino)methyl)pyrrolidin-2-one;
(S)-5-((((6-(2,2′-dichloro-3′-(2-(((2-hydroxyethyl)(methyl)amino)methyl)imidazo[1,2-b]pyridazin-6-yl)-[1,1′-biphenyl]-3-yl)-2-methoxypyridin-3-yl)methyl)amino)methyl)pyrrolidin-2-one;
(R)-5-((((6-(2,2′-dichloro-3′-(2-(((2-hydroxyethyl)(methyl)amino)methyl)imidazo[1,2-b]pyridazin-6-yl)-[1,1′-biphenyl]-3-yl)-2-methoxypyridin-3-yl)methyl)amino)methyl)pyrrolidin-2-one;
(S)-5-((((6-(2,2′-dichloro-3′-(2-((((S)-2-hydroxypropyl)amino)methyl)imidazo[1,2-b]pyridazin-6-yl)-[1,1′-biphenyl]-3-yl)-2-methoxypyridin-3-yl)methyl)amino)methyl)pyrrolidin-2-one;
(R)-5-((((6-(2,2′-dichloro-3′-(2-((((S)-2-hydroxypropyl)amino)methyl)imidazo[1,2-b]pyridazin-6-yl)-[1,1′-biphenyl]-3-yl)-2-methoxypyridin-3-yl)methyl)amino)methyl)pyrrolidin-2-one;
(S)-5-((((6-(2,2′-dichloro-3′-(2-((((R)-2-hydroxypropyl)amino)methyl)imidazo[1,2-b]pyridazin-6-yl)-[1,1′-biphenyl]-3-yl)-2-methoxypyridin-3-yl)methyl)amino)methyl)pyrrolidin-2-one;
(R)-5-((((6-(2,2′-dichloro-3′-(2-((((R)-2-hydroxypropyl)amino)methyl)imidazo[1,2-b]pyridazin-6-yl)-[1,1′-biphenyl]-3-yl)-2-methoxypyridin-3-yl)methyl)amino)methyl)pyrrolidin-2-one;
(S)-4-(((6-(2,2′-dichloro-3′-(6-methoxy-5-(((((S)-5-oxopyrrolidin-2-yl)methyl)amino)methyl)pyridin-2-yl)-[1,1′-biphenyl]-3-yl)imidazo[1,2-b]pyridazin-2-yl)methyl)amino)-3-hydroxy-N,N-dimethylbutanamide;
(R)-4-(((6-(2,2′-dichloro-3′-(6-methoxy-5-(((((S)-5-oxopyrrolidin-2-yl)methyl)amino)methyl)pyridin-2-yl)-[1,1′-biphenyl]-3-yl)imidazo[1,2-b]pyridazin-2-yl)methyl)amino)-3-hydroxy-N,N-dimethylbutanamide;
(S)-4-(((6-(2,2′-dichloro-3′-(6-methoxy-5-(((((R)-5-oxopyrrolidin-2-yl)methyl)amino)methyl)pyridin-2-yl)-[1,1′-biphenyl]-3-yl)imidazo[1,2-b]pyridazin-2-yl)methyl)amino)-3-hydroxy-N,N-dimethylbutanamide;
(R)-4-(((6-(2,2′-dichloro-3′-(6-methoxy-5-(((((R)-5-oxopyrrolidin-2-yl)methyl)amino)methyl)pyridin-2-yl)-[1,1′-biphenyl]-3-yl)imidazo[1,2-b]pyridazin-2-yl)methyl)amino)-3-hydroxy-N,N-dimethylbutanamide;
(S)-5-((((6-(2,2′-dichloro-3′-(2-(((((S)-5-oxopyrrolidin-2-yl)methyl)amino)methyl)imidazo[1,2-a]pyridin-6-yl)-[1,1′-biphenyl]-3-yl)-2-methoxypyridin-3-yl)methyl)amino)methyl)pyrrolidin-2-one;
(R)-5-((((6-(2,2′-dichloro-3′-(2-(((((S)-5-oxopyrrolidin-2-yl)methyl)amino)methyl)imidazo[1,2-a]pyridin-6-yl)-[1,1′-biphenyl]-3-yl)-2-methoxypyridin-3-yl)methyl)amino)methyl)pyrrolidin-2-one;
(S)-5-((((6-(2,2′-dichloro-3′-(2-(((((R)-5-oxopyrrolidin-2-yl)methyl)amino)methyl)imidazo[1,2-a]pyridin-6-yl)-[1,1′-biphenyl]-3-yl)-2-methoxypyridin-3-yl)methyl)amino)methyl)pyrrolidin-2-one;
(R)-5-((((6-(2,2′-dichloro-3′-(2-(((((R)-5-oxopyrrolidin-2-yl)methyl)amino)methyl)imidazo[1,2-a]pyridin-6-yl)-[1,1′-biphenyl]-3-yl)-2-methoxypyridin-3-yl)methyl)amino)methyl)pyrrolidin-2-one;
(S)-5-((((6-(2,2′-dichloro-3′-(2-(((((S)-5-oxopyrrolidin-2-yl)methyl)amino)methyl)imidazo[1,2-a]pyridin-7-yl)-[1,1′-biphenyl]-3-yl)-2-methoxypyridin-3-yl)methyl)amino)methyl)pyrrolidin-2-one;
(R)-5-((((6-(2,2′-dichloro-3′-(2-(((((S)-5-oxopyrrolidin-2-yl)methyl)amino)methyl)imidazo[1,2-a]pyridin-7-yl)-[1,1′-biphenyl]-3-yl)-2-methoxypyridin-3-yl)methyl)amino)methyl)pyrrolidin-2-one;
(S)-5-((((6-(2,2′-dichloro-3′-(2-(((((R)-5-oxopyrrolidin-2-yl)methyl)amino)methyl)imidazo[1,2-a]pyridin-7-yl)-[1,1′-biphenyl]-3-yl)-2-methoxypyridin-3-yl)methyl)amino)methyl)pyrrolidin-2-one;
(R)-5-((((6-(2,2′-dichloro-3′-(2-(((((R)-5-oxopyrrolidin-2-yl)methyl)amino)methyl)imidazo[1,2-a]pyridin-7-yl)-[1,1′-biphenyl]-3-yl)-2-methoxypyridin-3-yl)methyl)amino)methyl)pyrrolidin-2-one;
methyl (S)-3-((((8-(2,2′-dichloro-3′-(6-methoxy-5-((((5-oxopyrrolidin-2-yl)methyl)amino)methyl)pyridin-2-yl)-[1,1′-biphenyl]-3-yl)-4-oxo-4H-pyrido[1,2-a]pyrimidin-3-yl)methyl)amino)methyl)oxetane-3-carboxylate;
methyl (R)-3-((((8-(2,2′-dichloro-3′-(6-methoxy-5-((((5-oxopyrrolidin-2-yl)methyl)amino)methyl)pyridin-2-yl)-[1,1′-biphenyl]-3-yl)-4-oxo-4H-pyrido[1,2-a]pyrimidin-3-yl)methyl)amino)methyl)oxetane-3-carboxylate;
8-(2,2′-dichloro-3′-(6-methoxy-5-(((((S)-5-oxopyrrolidin-2-yl)methyl)amino)methyl)pyridin-2-yl)-[1,1′-biphenyl]-3-yl)-3-(((((R)-3,3-dimethyl-5-oxopyrrolidin-2-yl)methyl)amino)methyl)-4H-pyrido[1,2-a]pyrimidin-4-one;
8-(2,2′-dichloro-3′-(6-methoxy-5-(((((S)-5-oxopyrrolidin-2-yl)methyl)amino)methyl)pyridin-2-yl)-[1,1′-biphenyl]-3-yl)-3-(((((S)-3,3-dimethyl-5-oxopyrrolidin-2-yl)methyl)amino)methyl)-4H-pyrido[1,2-a]pyrimidin-4-one;
8-(2,2′-dichloro-3′-(6-methoxy-5-(((((R)-5-oxopyrrolidin-2-yl)methyl)amino)methyl)pyridin-2-yl)-[1,1′-biphenyl]-3-yl)-3-(((((R)-3,3-dimethyl-5-oxopyrrolidin-2-yl)methyl)amino)methyl)-4H-pyrido[1,2-a]pyrimidin-4-one;
8-(2,2′-dichloro-3′-(6-methoxy-5-(((((R)-5-oxopyrrolidin-2-yl)methyl)amino)methyl)pyridin-2-yl)-[1,1′-biphenyl]-3-yl)-3-(((((S)-3,3-dimethyl-5-oxopyrrolidin-2-yl)methyl)amino)methyl)-4H-pyrido[1,2-a]pyrimidin-4-one;
8-(2,2′-dichloro-3′-(5-methoxy-6-(((((S)-5-oxopyrrolidin-2-yl)methyl)amino)methyl)pyridin-3-yl)-[1,1′-biphenyl]-3-yl)-3-(((((S)-5-oxopyrrolidin-2-yl)methyl)amino)methyl)-4H-pyrido[1,2-a]pyrimidin-4-one;
8-(2,2′-dichloro-3′-(5-methoxy-6-(((((R)-5-oxopyrrolidin-2-yl)methyl)amino)methyl)pyridin-3-yl)-[1,1′-biphenyl]-3-yl)-3-(((((S)-5-oxopyrrolidin-2-yl)methyl)amino)methyl)-4H-pyrido[1,2-a]pyrimidin-4-one;
8-(2,2′-dichloro-3′-(5-methoxy-6-(((((S)-5-oxopyrrolidin-2-yl)methyl)amino)methyl)pyridin-3-yl)-[1,1′-biphenyl]-3-yl)-3-(((((R)-5-oxopyrrolidin-2-yl)methyl)amino)methyl)-4H-pyrido[1,2-a]pyrimidin-4-one;
8-(2,2′-dichloro-3′-(5-methoxy-6-(((((R)-5-oxopyrrolidin-2-yl)methyl)amino)methyl)pyridin-3-yl)-[1,1′-biphenyl]-3-yl)-3-(((((R)-5-oxopyrrolidin-2-yl)methyl)amino)methyl)-4H-pyrido[1,2-a]pyrimidin-4-one;
8-(2,2′-dichloro-3′-(6-((((1r,3r)-3-hydroxy-3-methylcyclobutyl)amino)methyl)-5-methoxypyridin-3-yl)-[1,1′-biphenyl]-3-yl)-3-((((1r,3r)-3-hydroxy-3-methylcyclobutyl)amino)methyl)-4H-pyrido[1,2-a]pyrimidin-4-one;
8-(2,2′-dichloro-3′-(6-((((1r,3r)-3-hydroxy-3-methylcyclobutyl)amino)methyl)-5-methoxypyridin-3-yl)-[1,1′-biphenyl]-3-yl)-3-((((1s,3s)-3-hydroxy-3-methylcyclobutyl)amino)methyl)-4H-pyrido[1,2-a]pyrimidin-4-one;
8-(2,2′-dichloro-3′-(6-((((1s,3s)-3-hydroxy-3-methylcyclobutyl)amino)methyl)-5-methoxypyridin-3-yl)-[1,1′-biphenyl]-3-yl)-3-((((1r,3r)-3-hydroxy-3-methylcyclobutyl)amino)methyl)-4H-pyrido[1,2-a]pyrimidin-4-one;
8-(2,2′-dichloro-3′-(6-((((1s,3s)-3-hydroxy-3-methylcyclobutyl)amino)methyl)-5-methoxypyridin-3-yl)-[1,1′-biphenyl]-3-yl)-3-((((1s,3s)-3-hydroxy-3-methylcyclobutyl)amino)methyl)-4H-pyrido[1,2-a]pyrimidin-4-one;
8-(2,2′-dichloro-3′-(6-(((2-hydroxyethyl)(methyl)amino)methyl)-5-methoxypyridin-3-yl)-[1,1′-biphenyl]-3-yl)-3-(((2-hydroxyethyl)(methyl)amino)methyl)-4H-pyrido[1,2-a]pyrimidin-4-one;
(S)-5-((((6-(2,2′-dichloro-3′-(1-methyl-2,3,4,5-tetrahydro-1H-benzo[e][1,4]diazepin-7-yl)-[1,1′-biphenyl]-3-yl)-2-methoxypyridin-3-yl)methyl)amino)methyl)pyrrolidin-2-one;
(R)-5-((((6-(2,2′-dichloro-3′-(1-methyl-2,3,4,5-tetrahydro-1H-benzo[e][1,4]diazepin-7-yl)-[1,1′-biphenyl]-3-yl)-2-methoxypyridin-3-yl)methyl)amino)methyl)pyrrolidin-2-one;
(S)-5-((((6-(2,2′-dichloro-3′-(1-methyl-2,3,4,5-tetrahydro-1H-benzo[e][1,4]diazepin-8-yl)-[1,1′-biphenyl]-3-yl)-2-methoxypyridin-3-yl)methyl)amino)methyl)pyrrolidin-2-one;
(R)-5-((((6-(2,2′-dichloro-3′-(1-methyl-2,3,4,5-tetrahydro-1H-benzo[e][1,4]diazepin-8-yl)-[1,1′-biphenyl]-3-yl)-2-methoxypyridin-3-yl)methyl)amino)methyl)pyrrolidin-2-one;
(S)-5-((((6-(2,2′-dichloro-3′-(1,2,3,4-tetrahydropyrazino[1,2-a]indol-8-yl)-[1,1′-biphenyl]-3-yl)-2-methoxypyridin-3-yl)methyl)amino)methyl)pyrrolidin-2-one;
(R)-5-((((6-(2,2′-dichloro-3′-(1,2,3,4-tetrahydropyrazino[1,2-a]indol-8-yl)-[1,1′-biphenyl]-3-yl)-2-methoxypyridin-3-yl)methyl)amino)methyl)pyrrolidin-2-one;
(S)-5-((((3-chloro-5-(2,2′-dichloro-3′-(6-methoxy-5-(((((S)-5-oxopyrrolidin-2-yl)methyl)amino)methyl)pyridin-2-yl)-[1,1′-biphenyl]-3-yl)-1-methyl-1H-pyrrolo[3,2-b]pyridin-2-yl)methyl)amino)methyl)pyrrolidin-2-one;
(R)-5-((((3-chloro-5-(2,2′-dichloro-3′-(6-methoxy-5-(((((S)-5-oxopyrrolidin-2-yl)methyl)amino)methyl)pyridin-2-yl)-[1,1′-biphenyl]-3-yl)-1-methyl-1H-pyrrolo[3,2-b]pyridin-2-yl)methyl)amino)methyl)pyrrolidin-2-one;
(S)-5-((((3-chloro-5-(2,2′-dichloro-3′-(6-methoxy-5-(((((R)-5-oxopyrrolidin-2-yl)methyl)amino)methyl)pyridin-2-yl)-[1,1′-biphenyl]-3-yl)-1-methyl-1H-pyrrolo[3,2-b]pyridin-2-yl)methyl)amino)methyl)pyrrolidin-2-one;
(R)-5-((((3-chloro-5-(2,2′-dichloro-3′-(6-methoxy-5-(((((R)-5-oxopyrrolidin-2-yl)methyl)amino)methyl)pyridin-2-yl)-[1,1′-biphenyl]-3-yl)-1-methyl-1H-pyrrolo[3,2-b]pyridin-2-yl)methyl)amino)methyl)pyrrolidin-2-one;
(S)-5-((((6-(2,2′-dichloro-3′-(8-chloro-6-((((R)-2-hydroxypropyl)amino)methyl)-5-methoxyquinolin-2-yl)-[1,1′-biphenyl]-3-yl)-2-methoxypyridin-3-yl)methyl)amino)methyl)pyrrolidin-2-one;
(R)-5-((((6-(2,2′-dichloro-3′-(8-chloro-6-((((R)-2-hydroxypropyl)amino)methyl)-5-methoxyquinolin-2-yl)-[1,1′-biphenyl]-3-yl)-2-methoxypyridin-3-yl)methyl)amino)methyl)pyrrolidin-2-one;
(S)-5-((((6-(2,2′-dichloro-3′-(8-chloro-6-((((S)-2-hydroxypropyl)amino)methyl)-5-methoxyquinolin-2-yl)-[1,1′-biphenyl]-3-yl)-2-methoxypyridin-3-yl)methyl)amino)methyl)pyrrolidin-2-one;
(R)-5-((((6-(2,2′-dichloro-3′-(8-chloro-6-((((S)-2-hydroxypropyl)amino)methyl)-5-methoxyquinolin-2-yl)-[1,1′-biphenyl]-3-yl)-2-methoxypyridin-3-yl)methyl)amino)methyl)pyrrolidin-2-one;
8-(2,2′-dichloro-3′-(6-methoxy-5-(((((S)-5-oxopyrrolidin-2-yl)methyl)amino)methyl)pyridin-2-yl)-[1,1′-biphenyl]-3-yl)-3-(((((2R)-methyl-5-oxopyrrolidin-2-yl)methyl)amino)methyl)-4H-pyrido[1,2-a]pyrimidin-4-one;
8-(2,2′-dichloro-3′-(6-methoxy-5-(((((S)-5-oxopyrrolidin-2-yl)methyl)amino)methyl)pyridin-2-yl)-[1,1′-biphenyl]-3-yl)-3-(((((2S)-methyl-5-oxopyrrolidin-2-yl)methyl)amino)methyl)-4H-pyrido[1,2-a]pyrimidin-4-one;
8-(2,2′-dichloro-3′-(6-methoxy-5-(((((R)-5-oxopyrrolidin-2-yl)methyl)amino)methyl)pyridin-2-yl)-[1,1′-biphenyl]-3-yl)-3-(((((2R)-methyl-5-oxopyrrolidin-2-yl)methyl)amino)methyl)-4H-pyrido[1,2-a]pyrimidin-4-one;
8-(2,2′-dichloro-3′-(6-methoxy-5-(((((R)-5-oxopyrrolidin-2-yl)methyl)amino)methyl)pyridin-2-yl)-[1,1′-biphenyl]-3-yl)-3-(((((2S)-methyl-5-oxopyrrolidin-2-yl)methyl)amino)methyl)-4H-pyrido[1,2-a]pyrimidin-4-one;
(S)-5-((((6-(2,2′-dichloro-3′-(6-(((((S)-5-oxopyrrolidin-2-yl)methyl)amino)methyl)quinolin-2-yl)-[1,1′-biphenyl]-3-yl)-2-methoxypyridin-3-yl)methyl)amino)methyl)pyrrolidin-2-one;
(R)-5-((((6-(2,2′-dichloro-3′-(6-(((((S)-5-oxopyrrolidin-2-yl)methyl)amino)methyl)quinolin-2-yl)-[1,1′-biphenyl]-3-yl)-2-methoxypyridin-3-yl)methyl)amino)methyl)pyrrolidin-2-one;
(S)-5-((((6-(2,2′-dichloro-3′-(6-(((((R)-5-oxopyrrolidin-2-yl)methyl)amino)methyl)quinolin-2-yl)-[1,1′-biphenyl]-3-yl)-2-methoxypyridin-3-yl)methyl)amino)methyl)pyrrolidin-2-one;
(R)-5-((((6-(2,2′-dichloro-3′-(6-(((((R)-5-oxopyrrolidin-2-yl)methyl)amino)methyl)quinolin-2-yl)-[1,1′-biphenyl]-3-yl)-2-methoxypyridin-3-yl)methyl)amino)methyl)pyrrolidin-2-one;
(S)-5-((((3-(2,2′-dichloro-3′-(6-methoxy-5-(((((S)-5-oxopyrrolidin-2-yl)methyl)amino)methyl)pyridin-2-yl)-[1,1′-biphenyl]-3-yl)quinolin-7-yl)methyl)amino)methyl)pyrrolidin-2-one;
(R)-5-((((3-(2,2′-dichloro-3′-(6-methoxy-5-(((((S)-5-oxopyrrolidin-2-yl)methyl)amino)methyl)pyridin-2-yl)-[1,1′-biphenyl]-3-yl)quinolin-7-yl)methyl)amino)methyl)pyrrolidin-2-one;
(S)-5-((((3-(2,2′-dichloro-3′-(6-methoxy-5-(((((R)-5-oxopyrrolidin-2-yl)methyl)amino)methyl)pyridin-2-yl)-[1,1′-biphenyl]-3-yl)quinolin-7-yl)methyl)amino)methyl)pyrrolidin-2-one;
(R)-5-((((3-(2,2′-dichloro-3′-(6-methoxy-5-(((((R)-5-oxopyrrolidin-2-yl)methyl)amino)methyl)pyridin-2-yl)-[1,1′-biphenyl]-3-yl)quinolin-7-yl)methyl)amino)methyl)pyrrolidin-2-one;
(S)-8-(2,2′-dichloro-3′-(6-methoxy-5-((((5-oxopyrrolidin-2-yl)methyl)amino)methyl)pyridin-2-yl)-[1,1′-biphenyl]-3-yl)-2-(((2-hydroxyethyl)amino)methyl)-3-methyl-4H-pyrido[1,2-a]pyrimidin-4-one;
(R)-8-(2,2′-dichloro-3′-(6-methoxy-5-((((5-oxopyrrolidin-2-yl)methyl)amino)methyl)pyridin-2-yl)-[1,1′-biphenyl]-3-yl)-2-(((2-hydroxyethyl)amino)methyl)-3-methyl-4H-pyrido[1,2-a]pyrimidin-4-one;
8-(2,2′-dichloro-3′-(6-methoxy-5-(((((S)-5-oxopyrrolidin-2-yl)methyl)amino)methyl)pyridin-2-yl)-[1,1′-biphenyl]-3-yl)-3-methyl-2-(((((S)-5-oxopyrrolidin-2-yl)methyl)amino)methyl)-4H-pyrido[1,2-a]pyrimidin-4-one;
8-(2,2′-dichloro-3′-(6-methoxy-5-(((((R)-5-oxopyrrolidin-2-yl)methyl)amino)methyl)pyridin-2-yl)-[1,1′-biphenyl]-3-yl)-3-methyl-2-(((((S)-5-oxopyrrolidin-2-yl)methyl)amino)methyl)-4H-pyrido[1,2-a]pyrimidin-4-one;
8-(2,2′-dichloro-3′-(6-methoxy-5-(((((S)-5-oxopyrrolidin-2-yl)methyl)amino)methyl)pyridin-2-yl)-[1,1′-biphenyl]-3-yl)-3-methyl-2-(((((R)-5-oxopyrrolidin-2-yl)methyl)amino)methyl)-4H-pyrido[1,2-a]pyrimidin-4-one;
8-(2,2′-dichloro-3′-(6-methoxy-5-(((((R)-5-oxopyrrolidin-2-yl)methyl)amino)methyl)pyridin-2-yl)-[1,1′-biphenyl]-3-yl)-3-methyl-2-(((((R)-5-oxopyrrolidin-2-yl)methyl)amino)methyl)-4H-pyrido[1,2-a]pyrimidin-4-one;
8-(2,2′-dichloro-3′-(6-methoxy-5-(((((S)-5-oxopyrrolidin-2-yl)methyl)amino)methyl)pyridin-2-yl)-[1,1′-biphenyl]-3-yl)-2-((((1r,3r)-3-hydroxy-3-methylcyclobutyl)amino)methyl)-3-methyl-4H-pyrido[1,2-a]pyrimidin-4-one;
8-(2,2′-dichloro-3′-(6-methoxy-5-(((((S)-5-oxopyrrolidin-2-yl)methyl)amino)methyl)pyridin-2-yl)-[1,1′-biphenyl]-3-yl)-2-((((1s,3s)-3-hydroxy-3-methylcyclobutyl)amino)methyl)-3-methyl-4H-pyrido[1,2-a]pyrimidin-4-one;
8-(2,2′-dichloro-3′-(6-methoxy-5-(((((R)-5-oxopyrrolidin-2-yl)methyl)amino)methyl)pyridin-2-yl)-[1,1′-biphenyl]-3-yl)-2-((((1r,3r)-3-hydroxy-3-methylcyclobutyl)amino)methyl)-3-methyl-4H-pyrido[1,2-a]pyrimidin-4-one;
8-(2,2′-dichloro-3′-(6-methoxy-5-(((((R)-5-oxopyrrolidin-2-yl)methyl)amino)methyl)pyridin-2-yl)-[1,1′-biphenyl]-3-yl)-2-((((1s,3s)-3-hydroxy-3-methylcyclobutyl)amino)methyl)-3-methyl-4H-pyrido[1,2-a]pyrimidin-4-one;
(S)-10-(2,2′-dichloro-3′-(6-methoxy-5-((((5-oxopyrrolidin-2-yl)methyl)amino)methyl)pyridin-2-yl)-[1,1′-biphenyl]-3-yl)-1-methyl-2,3,4,5-tetrahydropyrido[1′,2′:1,2]pyrimido[4,5-e][1,4]diazepin-6(1H)-one;
(R)-10-(2,2′-dichloro-3′-(6-methoxy-5-((((5-oxopyrrolidin-2-yl)methyl)amino)methyl)pyridin-2-yl)-[1,1′-biphenyl]-3-yl)-1-methyl-2,3,4,5-tetrahydropyrido[1′,2′:1,2]pyrimido[4,5-e][1,4]diazepin-6(1H)-one;
(S)-5-((((6-(2,2′-dichloro-3′-(1-methyl-3-(((((S)-5-oxopyrrolidin-2-yl)methyl)amino)methyl)-1H-pyrrolo[3,2-b]pyridin-6-yl)-[1,1′-biphenyl]-3-yl)-2-methoxypyridin-3-yl)methyl)amino)methyl)pyrrolidin-2-one;
(R)-5-((((6-(2,2′-dichloro-3′-(1-methyl-3-(((((S)-5-oxopyrrolidin-2-yl)methyl)amino)methyl)-1H-pyrrolo[3,2-b]pyridin-6-yl)-[1,1′-biphenyl]-3-yl)-2-methoxypyridin-3-yl)methyl)amino)methyl)pyrrolidin-2-one;
(S)-5-((((6-(2,2′-dichloro-3′-(1-methyl-3-(((((R)-5-oxopyrrolidin-2-yl)methyl)amino)methyl)-1H-pyrrolo[3,2-b]pyridin-6-yl)-[1,1′-biphenyl]-3-yl)-2-methoxypyridin-3-yl)methyl)amino)methyl)pyrrolidin-2-one;
(R)-5-((((6-(2,2′-dichloro-3′-(1-methyl-3-(((((R)-5-oxopyrrolidin-2-yl)methyl)amino)methyl)-1H-pyrrolo[3,2-b]pyridin-6-yl)-[1,1′-biphenyl]-3-yl)-2-methoxypyridin-3-yl)methyl)amino)methyl)pyrrolidin-2-one;
8-(2,2′-dichloro-3′-(6-methoxy-5-(((((S)-5-oxopyrrolidin-2-yl)methyl)amino)methyl)pyridin-2-yl)-[1,1′-biphenyl]-3-yl)-2-((((1r,3r)-3-hydroxy-3-methylcyclobutyl)amino)methyl)-4H-pyrido[1,2-a]pyrimidin-4-one;
8-(2,2′-dichloro-3′-(6-methoxy-5-(((((R)-5-oxopyrrolidin-2-yl)methyl)amino)methyl)pyridin-2-yl)-[1,1′-biphenyl]-3-yl)-2-((((1r,3r)-3-hydroxy-3-methylcyclobutyl)amino)methyl)-4H-pyrido[1,2-a]pyrimidin-4-one;
8-(2,2′-dichloro-3′-(6-methoxy-5-(((((S)-5-oxopyrrolidin-2-yl)methyl)amino)methyl)pyridin-2-yl)-[1,1′-biphenyl]-3-yl)-2-((((1s,3s)-3-hydroxy-3-methylcyclobutyl)amino)methyl)-4H-pyrido[1,2-a]pyrimidin-4-one;
8-(2,2′-dichloro-3′-(6-methoxy-5-(((((R)-5-oxopyrrolidin-2-yl)methyl)amino)methyl)pyridin-2-yl)-[1,1′-biphenyl]-3-yl)-2-((((1s,3s)-3-hydroxy-3-methylcyclobutyl)amino)methyl)-4H-pyrido[1,2-a]pyrimidin-4-one;
8-(2,2′-dichloro-3′-(6-methoxy-5-(((((S)-5-oxopyrrolidin-2-yl)methyl)amino)methyl)pyridin-2-yl)-[1,1′-biphenyl]-3-yl)-2-((((S)-2-hydroxypropyl)amino)methyl)-3-methyl-4H-pyrido[1,2-a]pyrimidin-4-one;
8-(2,2′-dichloro-3′-(6-methoxy-5-(((((R)-5-oxopyrrolidin-2-yl)methyl)amino)methyl)pyridin-2-yl)-[1,1′-biphenyl]-3-yl)-2-((((S)-2-hydroxypropyl)amino)methyl)-3-methyl-4H-pyrido[1,2-a]pyrimidin-4-one;
8-(2,2′-dichloro-3′-(6-methoxy-5-(((((S)-5-oxopyrrolidin-2-yl)methyl)amino)methyl)pyridin-2-yl)-[1,1′-biphenyl]-3-yl)-2-((((R)-2-hydroxypropyl)amino)methyl)-3-methyl-4H-pyrido[1,2-a]pyrimidin-4-one;
8-(2,2′-dichloro-3′-(6-methoxy-5-(((((R)-5-oxopyrrolidin-2-yl)methyl)amino)methyl)pyridin-2-yl)-[1,1′-biphenyl]-3-yl)-2-((((R)-2-hydroxypropyl)amino)methyl)-3-methyl-4H-pyrido[1,2-a]pyrimidin-4-one;
(S)-8-(2,2′-dichloro-3′-(4-chloro-6-methoxy-5-((((5-oxopyrrolidin-2-yl)methyl)amino)methyl)pyridin-2-yl)-[1,1′-biphenyl]-3-yl)-3-(((2-hydroxyethyl)amino)methyl)-4H-pyrido[1,2-a]pyrimidin-4-one;
(R)-8-(2,2′-dichloro-3′-(4-chloro-6-methoxy-5-((((5-oxopyrrolidin-2-yl)methyl)amino)methyl)pyridin-2-yl)-[1,1′-biphenyl]-3-yl)-3-(((2-hydroxyethyl)amino)methyl)-4H-pyrido[1,2-a]pyrimidin-4-one;
(S)-5-((((6-(2,2′-dichloro-3′-(2-methoxy-3-(((((S)-5-oxopyrrolidin-2-yl)methyl)amino)methyl)pyrazolo[1,5-a]pyrimidin-6-yl)-[1,1′-biphenyl]-3-yl)-2-methoxypyridin-3-yl)methyl)amino)methyl)pyrrolidin-2-one;
(R)-5-((((6-(2,2′-dichloro-3′-(2-methoxy-3-(((((S)-5-oxopyrrolidin-2-yl)methyl)amino)methyl)pyrazolo[1,5-a]pyrimidin-6-yl)-[1,1′-biphenyl]-3-yl)-2-methoxypyridin-3-yl)methyl)amino)methyl)pyrrolidin-2-one;
(S)-5-((((6-(2,2′-dichloro-3′-(2-methoxy-3-(((((R)-5-oxopyrrolidin-2-yl)methyl)amino)methyl)pyrazolo[1,5-a]pyrimidin-6-yl)-[1,1′-biphenyl]-3-yl)-2-methoxypyridin-3-yl)methyl)amino)methyl)pyrrolidin-2-one;
(R)-5-((((6-(2,2′-dichloro-3′-(2-methoxy-3-(((((R)-5-oxopyrrolidin-2-yl)methyl)amino)methyl)pyrazolo[1,5-a]pyrimidin-6-yl)-[1,1′-biphenyl]-3-yl)-2-methoxypyridin-3-yl)methyl)amino)methyl)pyrrolidin-2-one;
(S)-5-((((6-(2,2′-dichloro-3′-(3-(((((S)-5-oxopyrrolidin-2-yl)methyl)amino)methyl)imidazo[1,2-a]pyridin-7-yl)-[1,1′-biphenyl]-3-yl)-2-methoxypyridin-3-yl)methyl)amino)methyl)pyrrolidin-2-one;
(R)-5-((((6-(2,2′-dichloro-3′-(3-(((((S)-5-oxopyrrolidin-2-yl)methyl)amino)methyl)imidazo[1,2-a]pyridin-7-yl)-[1,1′-biphenyl]-3-yl)-2-methoxypyridin-3-yl)methyl)amino)methyl)pyrrolidin-2-one;
(S)-5-((((6-(2,2′-dichloro-3′-(3-(((((R)-5-oxopyrrolidin-2-yl)methyl)amino)methyl)imidazo[1,2-a]pyridin-7-yl)-[1,1′-biphenyl]-3-yl)-2-methoxypyridin-3-yl)methyl)amino)methyl)pyrrolidin-2-one;
(R)-5-((((6-(2,2′-dichloro-3′-(3-(((((R)-5-oxopyrrolidin-2-yl)methyl)amino)methyl)imidazo[1,2-a]pyridin-7-yl)-[1,1′-biphenyl]-3-yl)-2-methoxypyridin-3-yl)methyl)amino)methyl)pyrrolidin-2-one;
8-(2,2′-dichloro-3′-(3-(((((S)-5-oxopyrrolidin-2-yl)methyl)amino)methyl)pyrazolo[1,5-a]pyrimidin-6-yl)-[1,1′-biphenyl]-3-yl)-3-(((((S)-5-oxopyrrolidin-2-yl)methyl)amino)methyl)-4H-pyrido[1,2-a]pyrimidin-4-one;
8-(2,2′-dichloro-3′-(3-(((((R)-5-oxopyrrolidin-2-yl)methyl)amino)methyl)pyrazolo[1,5-a]pyrimidin-6-yl)-[1,1′-biphenyl]-3-yl)-3-(((((S)-5-oxopyrrolidin-2-yl)methyl)amino)methyl)-4H-pyrido[1,2-a]pyrimidin-4-one;
8-(2,2′-dichloro-3′-(3-(((((S)-5-oxopyrrolidin-2-yl)methyl)amino)methyl)pyrazolo[1,5-a]pyrimidin-6-yl)-[1,1′-biphenyl]-3-yl)-3-(((((R)-5-oxopyrrolidin-2-yl)methyl)amino)methyl)-4H-pyrido[1,2-a]pyrimidin-4-one;
8-(2,2′-dichloro-3′-(3-(((((R)-5-oxopyrrolidin-2-yl)methyl)amino)methyl)pyrazolo[1,5-a]pyrimidin-6-yl)-[1,1′-biphenyl]-3-yl)-3-(((((R)-5-oxopyrrolidin-2-yl)methyl)amino)methyl)-4H-pyrido[1,2-a]pyrimidin-4-one;
(S)-5-((((3-chloro-7-(2,2′-dichloro-3′-(6-methoxy-5-(((((S)-5-oxopyrrolidin-2-yl)methyl)amino)methyl)pyridin-2-yl)-[1,1′-biphenyl]-3-yl)imidazo[1,2-a]pyridin-2-yl)methyl)amino)methyl)pyrrolidin-2-one;
(R)-5-((((3-chloro-7-(2,2′-dichloro-3′-(6-methoxy-5-(((((S)-5-oxopyrrolidin-2-yl)methyl)amino)methyl)pyridin-2-yl)-[1,1′-biphenyl]-3-yl)imidazo[1,2-a]pyridin-2-yl)methyl)amino)methyl)pyrrolidin-2-one;
(S)-5-((((3-chloro-7-(2,2′-dichloro-3′-(6-methoxy-5-(((((R)-5-oxopyrrolidin-2-yl)methyl)amino)methyl)pyridin-2-yl)-[1,1′-biphenyl]-3-yl)imidazo[1,2-a]pyridin-2-yl)methyl)amino)methyl)pyrrolidin-2-one;
(R)-5-((((3-chloro-7-(2,2′-dichloro-3′-(6-methoxy-5-(((((R)-5-oxopyrrolidin-2-yl)methyl)amino)methyl)pyridin-2-yl)-[1,1′-biphenyl]-3-yl)imidazo[1,2-a]pyridin-2-yl)methyl)amino)methyl)pyrrolidin-2-one;
2-(((3-chloro-7-(2,2′-dichloro-3′-(5-(((2-hydroxyethyl)amino)methyl)-6-methoxypyridin-2-yl)-[1,1′-biphenyl]-3-yl)imidazo[1,2-a]pyridin-2-yl)methyl)amino)ethan-1-ol;
methyl (S)-3-(((8-(2,2′-dichloro-3′-(6-methoxy-5-((((5-oxopyrrolidin-2-yl)methyl)amino)methyl)pyridin-2-yl)-[1,1′-biphenyl]-3-yl)-4-oxo-4H-pyrido[1,2-a]pyrimidin-3-yl)methyl)amino)-2,2-dimethylpropanoate;
methyl (R)-3-(((8-(2,2′-dichloro-3′-(6-methoxy-5-((((5-oxopyrrolidin-2-yl)methyl)amino)methyl)pyridin-2-yl)-[1,1′-biphenyl]-3-yl)-4-oxo-4H-pyrido[1,2-a]pyrimidin-3-yl)methyl)amino)-2,2-dimethylpropanoate;
(S)-5-((((3-chloro-5-(2,2′-dichloro-3′-(6-methoxy-5-(((((S)-5-oxopyrrolidin-2-yl)methyl)amino)methyl)pyridin-2-yl)-[1,1′-biphenyl]-3-yl)-1H-pyrrolo[3,2-b]pyridin-2-yl)methyl)amino)methyl)pyrrolidin-2-one;
(R)-5-((((3-chloro-5-(2,2′-dichloro-3′-(6-methoxy-5-(((((S)-5-oxopyrrolidin-2-yl)methyl)amino)methyl)pyridin-2-yl)-[1,1′-biphenyl]-3-yl)-1H-pyrrolo[3,2-b]pyridin-2-yl)methyl)amino)methyl)pyrrolidin-2-one;
(S)-5-((((3-chloro-5-(2,2′-dichloro-3′-(6-methoxy-5-(((((R)-5-oxopyrrolidin-2-yl)methyl)amino)methyl)pyridin-2-yl)-[1,1′-biphenyl]-3-yl)-1H-pyrrolo[3,2-b]pyridin-2-yl)methyl)amino)methyl)pyrrolidin-2-one;
(R)-5-((((3-chloro-5-(2,2′-dichloro-3′-(6-methoxy-5-(((((R)-5-oxopyrrolidin-2-yl)methyl)amino)methyl)pyridin-2-yl)-[1,1′-biphenyl]-3-yl)-1H-pyrrolo[3,2-b]pyridin-2-yl)methyl)amino)methyl)pyrrolidin-2-one;
(S)-5-((((3-chloro-6-(2,2′-dichloro-3′-(6-methoxy-5-(((((S)-5-oxopyrrolidin-2-yl)methyl)amino)methyl)pyridin-2-yl)-[1,1′-biphenyl]-3-yl)-1H-pyrrolo[2,3-b]pyridin-2-yl)methyl)amino)methyl)pyrrolidin-2-one;
(R)-5-((((3-chloro-6-(2,2′-dichloro-3′-(6-methoxy-5-(((((S)-5-oxopyrrolidin-2-yl)methyl)amino)methyl)pyridin-2-yl)-[1,1′-biphenyl]-3-yl)-1H-pyrrolo[2,3-b]pyridin-2-yl)methyl)amino)methyl)pyrrolidin-2-one;
(S)-5-((((3-chloro-6-(2,2′-dichloro-3′-(6-methoxy-5-(((((R)-5-oxopyrrolidin-2-yl)methyl)amino)methyl)pyridin-2-yl)-[1,1′-biphenyl]-3-yl)-1H-pyrrolo[2,3-b]pyridin-2-yl)methyl)amino)methyl)pyrrolidin-2-one;
(R)-5-((((3-chloro-6-(2,2′-dichloro-3′-(6-methoxy-5-(((((R)-5-oxopyrrolidin-2-yl)methyl)amino)methyl)pyridin-2-yl)-[1,1′-biphenyl]-3-yl)-1H-pyrrolo[2,3-b]pyridin-2-yl)methyl)amino)methyl)pyrrolidin-2-one;
2-(2,2′-dichloro-3′-(6-methoxy-5-(((((S)-5-oxopyrrolidin-2-yl)methyl)amino)methyl)pyridin-2-yl)-[1,1′-biphenyl]-3-yl)-5-(((((S)-5-oxopyrrolidin-2-yl)methyl)amino)methyl)isoindolin-1-one;
2-(2,2′-dichloro-3′-(6-methoxy-5-(((((R)-5-oxopyrrolidin-2-yl)methyl)amino)methyl)pyridin-2-yl)-[1,1′-biphenyl]-3-yl)-5-(((((S)-5-oxopyrrolidin-2-yl)methyl)amino)methyl)isoindolin-1-one;
2-(2,2′-dichloro-3′-(6-methoxy-5-(((((S)-5-oxopyrrolidin-2-yl)methyl)amino)methyl)pyridin-2-yl)-[1,1′-biphenyl]-3-yl)-5-(((((R)-5-oxopyrrolidin-2-yl)methyl)amino)methyl)isoindolin-1-one;
2-(2,2′-dichloro-3′-(6-methoxy-5-(((((R)-5-oxopyrrolidin-2-yl)methyl)amino)methyl)pyridin-2-yl)-[1,1′-biphenyl]-3-yl)-5-(((((R)-5-oxopyrrolidin-2-yl)methyl)amino)methyl)isoindolin-1-one;
(S)-5-((((6-(2,2′-dichloro-3′-(1-methyl-3-(((((S)-5-oxopyrrolidin-2-yl)methyl)amino)methyl)-1H-pyrrolo[2,3-b]pyridin-6-yl)-[1,1′-biphenyl]-3-yl)-2-methoxypyridin-3-yl)methyl)amino)methyl)pyrrolidin-2-one;
(R)-5-((((6-(2,2′-dichloro-3′-(1-methyl-3-(((((S)-5-oxopyrrolidin-2-yl)methyl)amino)methyl)-1H-pyrrolo[2,3-b]pyridin-6-yl)-[1,1′-biphenyl]-3-yl)-2-methoxypyridin-3-yl)methyl)amino)methyl)pyrrolidin-2-one;
(S)-5-((((6-(2,2′-dichloro-3′-(1-methyl-3-(((((R)-5-oxopyrrolidin-2-yl)methyl)amino)methyl)-1H-pyrrolo[2,3-b]pyridin-6-yl)-[1,1′-biphenyl]-3-yl)-2-methoxypyridin-3-yl)methyl)amino)methyl)pyrrolidin-2-one;
(R)-5-((((6-(2,2′-dichloro-3′-(1-methyl-3-(((((R)-5-oxopyrrolidin-2-yl)methyl)amino)methyl)-1H-pyrrolo[2,3-b]pyridin-6-yl)-[1,1′-biphenyl]-3-yl)-2-methoxypyridin-3-yl)methyl)amino)methyl)pyrrolidin-2-one;
(S)-5-((((6-(2,2′-dichloro-3′-(3-(((((S)-5-oxopyrrolidin-2-yl)methyl)amino)methyl)imidazo[1,2-a]pyridin-6-yl)-[1,1′-biphenyl]-3-yl)-2-methoxypyridin-3-yl)methyl)amino)methyl)pyrrolidin-2-one;
(R)-5-((((6-(2,2′-dichloro-3′-(3-(((((S)-5-oxopyrrolidin-2-yl)methyl)amino)methyl)imidazo[1,2-a]pyridin-6-yl)-[1,1′-biphenyl]-3-yl)-2-methoxypyridin-3-yl)methyl)amino)methyl)pyrrolidin-2-one;
(S)-5-((((6-(2,2′-dichloro-3′-(3-(((((R)-5-oxopyrrolidin-2-yl)methyl)amino)methyl)imidazo[1,2-a]pyridin-6-yl)-[1,1′-biphenyl]-3-yl)-2-methoxypyridin-3-yl)methyl)amino)methyl)pyrrolidin-2-one;
(R)-5-((((6-(2,2′-dichloro-3′-(3-(((((R)-5-oxopyrrolidin-2-yl)methyl)amino)methyl)imidazo[1,2-a]pyridin-6-yl)-[1,1′-biphenyl]-3-yl)-2-methoxypyridin-3-yl)methyl)amino)methyl)pyrrolidin-2-one;
(5S)-5-((((6-(2,2′-dichloro-3′-((S)-5-(((S)-2-hydroxypropyl)amino)-5,6,7,8-tetrahydroquinolin-2-yl)-[1,1′-biphenyl]-3-yl)-2-methoxypyridin-3-yl)methyl)amino)methyl)pyrrolidin-2-one;
(5R)-5-((((6-(2,2′-dichloro-3′-((S)-5-(((S)-2-hydroxypropyl)amino)-5,6,7,8-tetrahydroquinolin-2-yl)-[1,1′-biphenyl]-3-yl)-2-methoxypyridin-3-yl)methyl)amino)methyl)pyrrolidin-2-one;
(5S)-5-((((6-(2,2′-dichloro-3′-((S)-5-(((R)-2-hydroxypropyl)amino)-5,6,7,8-tetrahydroquinolin-2-yl)-[1,1′-biphenyl]-3-yl)-2-methoxypyridin-3-yl)methyl)amino)methyl)pyrrolidin-2-one;
(5R)-5-((((6-(2,2′-dichloro-3′-((S)-5-(((R)-2-hydroxypropyl)amino)-5,6,7,8-tetrahydroquinolin-2-yl)-[1,1′-biphenyl]-3-yl)-2-methoxypyridin-3-yl)methyl)amino)methyl)pyrrolidin-2-one;
(5S)-5-((((6-(2,2′-dichloro-3′-((R)-5-(((S)-2-hydroxypropyl)amino)-5,6,7,8-tetrahydroquinolin-2-yl)-[1,1′-biphenyl]-3-yl)-2-methoxypyridin-3-yl)methyl)amino)methyl)pyrrolidin-2-one;
(5R)-5-((((6-(2,2′-dichloro-3′-((R)-5-(((S)-2-hydroxypropyl)amino)-5,6,7,8-tetrahydroquinolin-2-yl)-[1,1′-biphenyl]-3-yl)-2-methoxypyridin-3-yl)methyl)amino)methyl)pyrrolidin-2-one;
(5S)-5-((((6-(2,2′-dichloro-3′-((R)-5-(((R)-2-hydroxypropyl)amino)-5,6,7,8-tetrahydroquinolin-2-yl)-[1,1′-biphenyl]-3-yl)-2-methoxypyridin-3-yl)methyl)amino)methyl)pyrrolidin-2-one;
(5R)-5-((((6-(2,2′-dichloro-3′-((R)-5-(((R)-2-hydroxypropyl)amino)-5,6,7,8-tetrahydroquinolin-2-yl)-[1,1′-biphenyl]-3-yl)-2-methoxypyridin-3-yl)methyl)amino)methyl)pyrrolidin-2-one;
(R)-2-(3-(6-(2,2′-dichloro-3′-(6-methoxy-5-((((5-oxopyrrolidin-2-yl)methyl)amino)methyl)pyridin-2-yl)-[1,1′-biphenyl]-3-yl)-3-oxo-[1,2,4]triazolo[4,3-a]pyridin-2(3H)-yl)propyl)isoindoline-1,3-dione;
(S)-2-(3-(6-(2,2′-dichloro-3′-(6-methoxy-5-((((5-oxopyrrolidin-2-yl)methyl)amino)methyl)pyridin-2-yl)-[1,1′-biphenyl]-3-yl)-3-oxo-[1,2,4]triazolo[4,3-a]pyridin-2(3H)-yl)propyl)isoindoline-1,3-dione;
(S)-6-(2,2′-dichloro-3′-(6-methoxy-5-((((5-oxopyrrolidin-2-yl)methyl)amino)methyl)pyridin-2-yl)-[1,1′-biphenyl]-3-yl)-2-(3-(diisobutylamino)propyl)-[1,2,4]triazolo[4,3-a]pyridin-3(2H)-one;
(R)-6-(2,2′-dichloro-3′-(6-methoxy-5-((((5-oxopyrrolidin-2-yl)methyl)amino)methyl)pyridin-2-yl)-[1,1′-biphenyl]-3-yl)-2-(3-(diisobutylamino)propyl)-[1,2,4]triazolo[4,3-a]pyridin-3(2H)-one;
8-(2,2′-dichloro-3′-(4-chloro-6-methoxy-5-(((((S)-5-oxopyrrolidin-2-yl)methyl)amino)methyl)pyridin-2-yl)-[1,1′-biphenyl]-3-yl)-3-((((1r,3r)-3-hydroxy-3-methylcyclobutyl)amino)methyl)-4H-pyrido[1,2-a]pyrimidin-4-one;
8-(2,2′-dichloro-3′-(4-chloro-6-methoxy-5-(((((S)-5-oxopyrrolidin-2-yl)methyl)amino)methyl)pyridin-2-yl)-[1,1′-biphenyl]-3-yl)-3-((((1s,3s)-3-hydroxy-3-methylcyclobutyl)amino)methyl)-4H-pyrido[1,2-a]pyrimidin-4-one;
8-(2,2′-dichloro-3′-(4-chloro-6-methoxy-5-(((((R)-5-oxopyrrolidin-2-yl)methyl)amino)methyl)pyridin-2-yl)-[1,1′-biphenyl]-3-yl)-3-((((1r,3r)-3-hydroxy-3-methylcyclobutyl)amino)methyl)-4H-pyrido[1,2-a]pyrimidin-4-one;
8-(2,2′-dichloro-3′-(4-chloro-6-methoxy-5-(((((R)-5-oxopyrrolidin-2-yl)methyl)amino)methyl)pyridin-2-yl)-[1,1′-biphenyl]-3-yl)-3-((((1s,3s)-3-hydroxy-3-methylcyclobutyl)amino)methyl)-4H-pyrido[1,2-a]pyrimidin-4-one;
(5S,5'S)-5,5′-(((2,2′-dichloro-[1,1′-biphenyl]-3,3′-diyl)bis(8-methoxy-3,4-dihydroisoquinoline-6,2(1H)-diyl))bis(methylene))bis(pyrrolidin-2-one);
(5R,5'S)-5,5′-(((2,2′-dichloro-[1,1′-biphenyl]-3,3′-diyl)bis(8-methoxy-3,4-dihydroisoquinoline-6,2(1H)-diyl))bis(methylene))bis(pyrrolidin-2-one);
(5S,5′R)-5,5′-(((2,2′-dichloro-[1,1′-biphenyl]-3,3′-diyl)bis(8-methoxy-3,4-dihydroisoquinoline-6,2(1H)-diyl))bis(methylene))bis(pyrrolidin-2-one);
(5R,5′R)-5,5′-(((2,2′-dichloro-[1,1′-biphenyl]-3,3′-diyl)bis(8-methoxy-3,4-dihydroisoquinoline-6,2(1H)-diyl))bis(methylene))bis(pyrrolidin-2-one);
(S)-6-(2,2′-dichloro-3′-(6-methoxy-5-((((5-oxopyrrolidin-2-yl)methyl)amino)methyl)pyridin-2-yl)-[1,1′-biphenyl]-3-yl)-2-(((2-hydroxyethyl)(methyl)amino)methyl)-3-methylpyrrolo[2,1-f][1,2,4]triazin-4(3H)-one;
(r)-6-(2,2′-dichloro-3′-(6-methoxy-5-((((5-oxopyrrolidin-2-yl)methyl)amino)methyl)pyridin-2-yl)-[1,1′-biphenyl]-3-yl)-2-(((2-hydroxyethyl)(methyl)amino)methyl)-3-methylpyrrolo[2,1-f][1,2,4]triazin-4(3H)-one;
(S)-10-(2,2′-dichloro-3′-(6-methoxy-5-((((5-oxopyrrolidin-2-yl)methyl)amino)methyl)pyridin-2-yl)-[1,1′-biphenyl]-3-yl)-2,3,4,5-tetrahydro-6H-pyrido[1′,2′:1,2]pyrimido[5,4-f][1,4]oxazepin-6-one;
(R)-10-(2,2′-dichloro-3′-(6-methoxy-5-((((5-oxopyrrolidin-2-yl)methyl)amino)methyl)pyridin-2-yl)-[1,1′-biphenyl]-3-yl)-2,3,4,5-tetrahydro-6H-pyrido[1′,2′:1,2]pyrimido[5,4-f][1,4]oxazepin-6-one;
(S)-5-((((6-(2,2′-dichloro-3′-(4-methoxy-3-(((((S)-5-oxopyrrolidin-2-yl)methyl)amino)methyl)pyrazolo[1,5-a]pyridin-6-yl)-[1,1′-biphenyl]-3-yl)-2-methoxypyridin-3-yl)methyl)amino)methyl)pyrrolidin-2-one;
(R)-5-((((6-(2,2′-dichloro-3′-(4-methoxy-3-(((((S)-5-oxopyrrolidin-2-yl)methyl)amino)methyl)pyrazolo[1,5-a]pyridin-6-yl)-[1,1′-biphenyl]-3-yl)-2-methoxypyridin-3-yl)methyl)amino)methyl)pyrrolidin-2-one;
(S)-5-((((6-(2,2′-dichloro-3′-(4-methoxy-3-(((((R)-5-oxopyrrolidin-2-yl)methyl)amino)methyl)pyrazolo[1,5-a]pyridin-6-yl)-[1,1′-biphenyl]-3-yl)-2-methoxypyridin-3-yl)methyl)amino)methyl)pyrrolidin-2-one;
(R)-5-((((6-(2,2′-dichloro-3′-(4-methoxy-3-(((((R)-5-oxopyrrolidin-2-yl)methyl)amino)methyl)pyrazolo[1,5-a]pyridin-6-yl)-[1,1′-biphenyl]-3-yl)-2-methoxypyridin-3-yl)methyl)amino)methyl)pyrrolidin-2-one;
(S)-5-((((8-chloro-2-(2,2′-dichloro-3′-(4-fluoro-6-methoxy-5-(((((S)-5-oxopyrrolidin-2-yl)methyl)amino)methyl)pyridin-2-yl)-[1,1′-biphenyl]-3-yl)-5-methoxyquinolin-6-yl)methyl)amino)methyl)pyrrolidin-2-one;
(R)-5-((((8-chloro-2-(2,2′-dichloro-3′-(4-fluoro-6-methoxy-5-(((((S)-5-oxopyrrolidin-2-yl)methyl)amino)methyl)pyridin-2-yl)-[1,1′-biphenyl]-3-yl)-5-methoxyquinolin-6-yl)methyl)amino)methyl)pyrrolidin-2-one;
(S)-5-((((8-chloro-2-(2,2′-dichloro-3′-(4-fluoro-6-methoxy-5-(((((R)-5-oxopyrrolidin-2-yl)methyl)amino)methyl)pyridin-2-yl)-[1,1′-biphenyl]-3-yl)-5-methoxyquinolin-6-yl)methyl)amino)methyl)pyrrolidin-2-one;
(R)-5-((((8-chloro-2-(2,2′-dichloro-3′-(4-fluoro-6-methoxy-5-(((((R)-5-oxopyrrolidin-2-yl)methyl)amino)methyl)pyridin-2-yl)-[1,1′-biphenyl]-3-yl)-5-methoxyquinolin-6-yl)methyl)amino)methyl)pyrrolidin-2-one;
8-(2,2′-dichloro-3′-(6-methoxy-5-(((((S)-5-oxopyrrolidin-2-yl)methyl)amino)methyl)pyridin-2-yl)-[1,1′-biphenyl]-3-yl)-2-ethyl-3-(((((S)-5-oxopyrrolidin-2-yl)methyl)amino)methyl)-4H-pyrido[1,2-a]pyrimidin-4-one;
8-(2,2′-dichloro-3′-(6-methoxy-5-(((((R)-5-oxopyrrolidin-2-yl)methyl)amino)methyl)pyridin-2-yl)-[1,1′-biphenyl]-3-yl)-2-ethyl-3-(((((S)-5-oxopyrrolidin-2-yl)methyl)amino)methyl)-4H-pyrido[1,2-a]pyrimidin-4-one;
8-(2,2′-dichloro-3′-(6-methoxy-5-(((((S)-5-oxopyrrolidin-2-yl)methyl)amino)methyl)pyridin-2-yl)-[1,1′-biphenyl]-3-yl)-2-ethyl-3-(((((R)-5-oxopyrrolidin-2-yl)methyl)amino)methyl)-4H-pyrido[1,2-a]pyrimidin-4-one;
8-(2,2′-dichloro-3′-(6-methoxy-5-(((((R)-5-oxopyrrolidin-2-yl)methyl)amino)methyl)pyridin-2-yl)-[1,1′-biphenyl]-3-yl)-2-ethyl-3-(((((R)-5-oxopyrrolidin-2-yl)methyl)amino)methyl)-4H-pyrido[1,2-a]pyrimidin-4-one;
6-(2,2′-dichloro-3′-(6-methoxy-5-(((((S)-5-oxopyrrolidin-2-yl)methyl)amino)methyl)pyridin-2-yl)-[1,1′-biphenyl]-3-yl)-8-fluoro-2-(2-((((S)-5-oxopyrrolidin-2-yl)methyl)amino)ethyl)-3,4-dihydroisoquinolin-1(2H)-one;
6-(2,2′-dichloro-3′-(6-methoxy-5-(((((R)-5-oxopyrrolidin-2-yl)methyl)amino)methyl)pyridin-2-yl)-[1,1′-biphenyl]-3-yl)-8-fluoro-2-(2-((((S)-5-oxopyrrolidin-2-yl)methyl)amino)ethyl)-3,4-dihydroisoquinolin-1(2H)-one;
6-(2,2′-dichloro-3′-(6-methoxy-5-(((((S)-5-oxopyrrolidin-2-yl)methyl)amino)methyl)pyridin-2-yl)-[1,1′-biphenyl]-3-yl)-8-fluoro-2-(2-((((R)-5-oxopyrrolidin-2-yl)methyl)amino)ethyl)-3,4-dihydroisoquinolin-1(2H)-one;
6-(2,2′-dichloro-3′-(6-methoxy-5-(((((R)-5-oxopyrrolidin-2-yl)methyl)amino)methyl)pyridin-2-yl)-[1,1′-biphenyl]-3-yl)-8-fluoro-2-(2-((((R)-5-oxopyrrolidin-2-yl)methyl)amino)ethyl)-3,4-dihydroisoquinolin-1(2H)-one;
(S)-5-((((2-(2,2′-dichloro-3′-(6-methoxy-5-(((((S)-5-oxopyrrolidin-2-yl)methyl)amino)methyl)pyridin-2-yl)-[1,1′-biphenyl]-3-yl)-7-methyl-7H-pyrrolo[2,3-d]pyrimidin-5-yl)methyl)amino)methyl)pyrrolidin-2-one;
(R)-5-((((2-(2,2′-dichloro-3′-(6-methoxy-5-(((((S)-5-oxopyrrolidin-2-yl)methyl)amino)methyl)pyridin-2-yl)-[1,1′-biphenyl]-3-yl)-7-methyl-7H-pyrrolo[2,3-d]pyrimidin-5-yl)methyl)amino)methyl)pyrrolidin-2-one;
(S)-5-((((2-(2,2′-dichloro-3′-(6-methoxy-5-(((((R)-5-oxopyrrolidin-2-yl)methyl)amino)methyl)pyridin-2-yl)-[1,1′-biphenyl]-3-yl)-7-methyl-7H-pyrrolo[2,3-d]pyrimidin-5-yl)methyl)amino)methyl)pyrrolidin-2-one;
(R)-5-((((2-(2,2′-dichloro-3′-(6-methoxy-5-(((((R)-5-oxopyrrolidin-2-yl)methyl)amino)methyl)pyridin-2-yl)-[1,1′-biphenyl]-3-yl)-7-methyl-7H-pyrrolo[2,3-d]pyrimidin-5-yl)methyl)amino)methyl)pyrrolidin-2-one;
(S)-5-((((6-(2,2′-dichloro-3′-(1-methyl-3-(((((S)-5-oxopyrrolidin-2-yl)methyl)amino)methyl)-1H-pyrrolo[3,2-c]pyridin-6-yl)-[1,1′-biphenyl]-3-yl)-2-methoxypyridin-3-yl)methyl)amino)methyl)pyrrolidin-2-one;
(R)-5-((((6-(2,2′-dichloro-3′-(1-methyl-3-(((((S)-5-oxopyrrolidin-2-yl)methyl)amino)methyl)-1H-pyrrolo[3,2-c]pyridin-6-yl)-[1,1′-biphenyl]-3-yl)-2-methoxypyridin-3-yl)methyl)amino)methyl)pyrrolidin-2-one;
(S)-5-((((6-(2,2′-dichloro-3′-(1-methyl-3-(((((R)-5-oxopyrrolidin-2-yl)methyl)amino)methyl)-1H-pyrrolo[3,2-c]pyridin-6-yl)-[1,1′-biphenyl]-3-yl)-2-methoxypyridin-3-yl)methyl)amino)methyl)pyrrolidin-2-one;
(R)-5-((((6-(2,2′-dichloro-3′-(1-methyl-3-(((((R)-5-oxopyrrolidin-2-yl)methyl)amino)methyl)-1H-pyrrolo[3,2-c]pyridin-6-yl)-[1,1′-biphenyl]-3-yl)-2-methoxypyridin-3-yl)methyl)amino)methyl)pyrrolidin-2-one;
(S)-7-(2,2′-dichloro-3′-(6-methoxy-5-((((5-oxopyrrolidin-2-yl)methyl)amino)methyl)pyridin-2-yl)-[1,1′-biphenyl]-3-yl)-1,3,4,5-tetrahydro-2H-pyrido[2,3-e][1,4]diazepin-2-one;
(R)-7-(2,2′-dichloro-3′-(6-methoxy-5-((((5-oxopyrrolidin-2-yl)methyl)amino)methyl)pyridin-2-yl)-[1,1′-biphenyl]-3-yl)-1,3,4,5-tetrahydro-2H-pyrido[2,3-e][1,4]diazepin-2-one;
8-(2,2′-dichloro-3′-(4-fluoro-6-methoxy-5-(((((S)-5-oxopyrrolidin-2-yl)methyl)amino)methyl)pyridin-2-yl)-[1,1′-biphenyl]-3-yl)-2-methyl-3-(((((S)-5-oxopyrrolidin-2-yl)methyl)amino)methyl)-4H-pyrido[1,2-a]pyrimidin-4-one;
8-(2,2′-dichloro-3′-(4-fluoro-6-methoxy-5-(((((R)-5-oxopyrrolidin-2-yl)methyl)amino)methyl)pyridin-2-yl)-[1,1′-biphenyl]-3-yl)-2-methyl-3-(((((S)-5-oxopyrrolidin-2-yl)methyl)amino)methyl)-4H-pyrido[1,2-a]pyrimidin-4-one;
8-(2,2′-dichloro-3′-(4-fluoro-6-methoxy-5-(((((S)-5-oxopyrrolidin-2-yl)methyl)amino)methyl)pyridin-2-yl)-[1,1′-biphenyl]-3-yl)-2-methyl-3-(((((R)-5-oxopyrrolidin-2-yl)methyl)amino)methyl)-4H-pyrido[1,2-a]pyrimidin-4-one;
8-(2,2′-dichloro-3′-(4-fluoro-6-methoxy-5-(((((R)-5-oxopyrrolidin-2-yl)methyl)amino)methyl)pyridin-2-yl)-[1,1′-biphenyl]-3-yl)-2-methyl-3-(((((R)-5-oxopyrrolidin-2-yl)methyl)amino)methyl)-4H-pyrido[1,2-a]pyrimidin-4-one;
(S)-6-(2,2′-dichloro-3′-(6-methoxy-5-((((5-oxopyrrolidin-2-yl)methyl)amino)methyl)pyridin-2-yl)-[1,1′-biphenyl]-3-yl)-2-(((2-hydroxy-2-methylpropyl)amino)methyl)-3-methylpyrrolo[2,1-f][1,2,4]triazin-4(3H)-one;
(R)-6-(2,2′-dichloro-3′-(6-methoxy-5-((((5-oxopyrrolidin-2-yl)methyl)amino)methyl)pyridin-2-yl)-[1,1′-biphenyl]-3-yl)-2-(((2-hydroxy-2-methylpropyl)amino)methyl)-3-methylpyrrolo[2,1-f][1,2,4]triazin-4(3H)-one;
(S)-6-(2,2′-dichloro-3′-(6-methoxy-5-((((5-oxopyrrolidin-2-yl)methyl)amino)methyl)pyridin-2-yl)-[1,1′-biphenyl]-3-yl)-3-methyl-2-(((3,3,3-trifluoropropyl)amino)methyl)pyrrolo[2,1-f][1,2,4]triazin-4(3H)-one;
(R)-6-(2,2′-dichloro-3′-(6-methoxy-5-((((5-oxopyrrolidin-2-yl)methyl)amino)methyl)pyridin-2-yl)-[1,1′-biphenyl]-3-yl)-3-methyl-2-(((3,3,3-trifluoropropyl)amino)methyl)pyrrolo[2,1-f][1,2,4]triazin-4(3H)-one;
(S)-8-(2,2′-dichloro-3′-(1-methyl-2,3,4,5-tetrahydro-1H-benzo[e][1,4]diazepin-8-yl)-[1,1′-biphenyl]-3-yl)-3-((((5-oxopyrrolidin-2-yl)methyl)amino)methyl)-4H-pyrido[1,2-a]pyrimidin-4-one;
(R)-8-(2,2′-dichloro-3′-(1-methyl-2,3,4,5-tetrahydro-1H-benzo[e][1,4]diazepin-8-yl)-[1,1′-biphenyl]-3-yl)-3-((((5-oxopyrrolidin-2-yl)methyl)amino)methyl)-4H-pyrido[1,2-a]pyrimidin-4-one;
(S)-5-((((6-(2,2′-dichloro-3′-(3-((((S)-2-hydroxypropyl)amino)methyl)-1-methyl-1H-pyrrolo[2,3-b]pyridin-6-yl)-[1,1′-biphenyl]-3-yl)-2-methoxypyridin-3-yl)methyl)amino)methyl)pyrrolidin-2-one;
(R)-5-((((6-(2,2′-dichloro-3′-(3-((((S)-2-hydroxypropyl)amino)methyl)-1-methyl-1H-pyrrolo[2,3-b]pyridin-6-yl)-[1,1′-biphenyl]-3-yl)-2-methoxypyridin-3-yl)methyl)amino)methyl)pyrrolidin-2-one;
(S)-5-((((6-(2,2′-dichloro-3′-(3-((((R)-2-hydroxypropyl)amino)methyl)-1-methyl-1H-pyrrolo[2,3-b]pyridin-6-yl)-[1,1′-biphenyl]-3-yl)-2-methoxypyridin-3-yl)methyl)amino)methyl)pyrrolidin-2-one;
(R)-5-((((6-(2,2′-dichloro-3′-(3-((((R)-2-hydroxypropyl)amino)methyl)-1-methyl-1H-pyrrolo[2,3-b]pyridin-6-yl)-[1,1′-biphenyl]-3-yl)-2-methoxypyridin-3-yl)methyl)amino)methyl)pyrrolidin-2-one;
(S)-5-((((6-(2,2′-dichloro-3′-(3-(((2-hydroxyethyl)(methyl)amino)methyl)-1-methyl-1H-pyrrolo[2,3-b]pyridin-6-yl)-[1,1′-biphenyl]-3-yl)-2-methoxypyridin-3-yl)methyl)amino)methyl)pyrrolidin-2-one;
(R)-5-((((6-(2,2′-dichloro-3′-(3-(((2-hydroxyethyl)(methyl)amino)methyl)-1-methyl-1H-pyrrolo[2,3-b]pyridin-6-yl)-[1,1′-biphenyl]-3-yl)-2-methoxypyridin-3-yl)methyl)amino)methyl)pyrrolidin-2-one;
(S)-5-((((6-(2,2′-dichloro-3′-(1-methyl-3-(((((S)-oxetan-2-yl)methyl)amino)methyl)-1H-pyrrolo[2,3-b]pyridin-6-yl)-[1,1′-biphenyl]-3-yl)-2-methoxypyridin-3-yl)methyl)amino)methyl)pyrrolidin-2-one;
(R)-5-((((6-(2,2′-dichloro-3′-(1-methyl-3-(((((S)-oxetan-2-yl)methyl)amino)methyl)-1H-pyrrolo[2,3-b]pyridin-6-yl)-[1,1′-biphenyl]-3-yl)-2-methoxypyridin-3-yl)methyl)amino)methyl)pyrrolidin-2-one;
(S)-5-((((6-(2,2′-dichloro-3′-(1-methyl-3-(((((R)-oxetan-2-yl)methyl)amino)methyl)-1H-pyrrolo[2,3-b]pyridin-6-yl)-[1,1′-biphenyl]-3-yl)-2-methoxypyridin-3-yl)methyl)amino)methyl)pyrrolidin-2-one;
(R)-5-((((6-(2,2′-dichloro-3′-(1-methyl-3-(((((R)-oxetan-2-yl)methyl)amino)methyl)-1H-pyrrolo[2,3-b]pyridin-6-yl)-[1,1′-biphenyl]-3-yl)-2-methoxypyridin-3-yl)methyl)amino)methyl)pyrrolidin-2-one;
(S)-5-((((6-(2,2′-dichloro-3′-(1-methyl-3-(((2-(2-oxopyrrolidin-1-yl)ethyl)amino)methyl)-1H-pyrrolo[2,3-b]pyridin-6-yl)-[1,1′-biphenyl]-3-yl)-2-methoxypyridin-3-yl)methyl)amino)methyl)pyrrolidin-2-one;
(R)-5-((((6-(2,2′-dichloro-3′-(1-methyl-3-(((2-(2-oxopyrrolidin-1-yl)ethyl)amino)methyl)-1H-pyrrolo[2,3-b]pyridin-6-yl)-[1,1′-biphenyl]-3-yl)-2-methoxypyridin-3-yl)methyl)amino)methyl)pyrrolidin-2-one;
(S)-5-((((6-(3′-(3-(((1-acetylpiperidin-4-yl)amino)methyl)-1-methyl-1H-pyrrolo[2,3-b]pyridin-6-yl)-2,2′-dichloro-[1,1′-biphenyl]-3-yl)-2-methoxypyridin-3-yl)methyl)amino)methyl)pyrrolidin-2-one;
(R)-5-((((6-(3′-(3-(((1-acetylpiperidin-4-yl)amino)methyl)-1-methyl-1H-pyrrolo[2,3-b]pyridin-6-yl)-2,2′-dichloro-[1,1′-biphenyl]-3-yl)-2-methoxypyridin-3-yl)methyl)amino)methyl)pyrrolidin-2-one;
8-(2,2′-dichloro-3′-(1-methyl-3-(((((S)-5-oxopyrrolidin-2-yl)methyl)amino)methyl)-1H-pyrrolo[2,3-b]pyridin-6-yl)-[1,1′-biphenyl]-3-yl)-3-(((((S)-5-oxopyrrolidin-2-yl)methyl)amino)methyl)-4H-pyrido[1,2-a]pyrimidin-4-one;
8-(2,2′-dichloro-3′-(1-methyl-3-(((((R)-5-oxopyrrolidin-2-yl)methyl)amino)methyl)-1H-pyrrolo[2,3-b]pyridin-6-yl)-[1,1′-biphenyl]-3-yl)-3-(((((S)-5-oxopyrrolidin-2-yl)methyl)amino)methyl)-4H-pyrido[1,2-a]pyrimidin-4-one;
8-(2,2′-dichloro-3′-(1-methyl-3-(((((S)-5-oxopyrrolidin-2-yl)methyl)amino)methyl)-1H-pyrrolo[2,3-b]pyridin-6-yl)-[1,1′-biphenyl]-3-yl)-3-(((((R)-5-oxopyrrolidin-2-yl)methyl)amino)methyl)-4H-pyrido[1,2-a]pyrimidin-4-one;
8-(2,2′-dichloro-3′-(1-methyl-3-(((((R)-5-oxopyrrolidin-2-yl)methyl)amino)methyl)-1H-pyrrolo[2,3-b]pyridin-6-yl)-[1,1′-biphenyl]-3-yl)-3-(((((R)-5-oxopyrrolidin-2-yl)methyl)amino)methyl)-4H-pyrido[1,2-a]pyrimidin-4-one;
(5S,5'S)-5,5′-(((((2,2′-dichloro-[1,1′-biphenyl]-3,3′-diyl)bis(1-methyl-1H-pyrrolo[2,3-b]pyridine-6,3-diyl))bis(methylene))bis(azanediyl))bis(methylene))bis(pyrrolidin-2-one);
(5R,5'S)-5,5′-(((((2,2′-dichloro-[1,1′-biphenyl]-3,3′-diyl)bis(1-methyl-1H-pyrrolo[2,3-b]pyridine-6,3-diyl))bis(methylene))bis(azanediyl))bis(methylene))bis(pyrrolidin-2-one);
(5S,5′R)-5,5′-(((((2,2′-dichloro-[1,1′-biphenyl]-3,3′-diyl)bis(1-methyl-1H-pyrrolo[2,3-b]pyridine-6,3-diyl))bis(methylene))bis(azanediyl))bis(methylene))bis(pyrrolidin-2-one);
(5R,5′R)-5,5′-(((((2,2′-dichloro-[1,1′-biphenyl]-3,3′-diyl)bis(1-methyl-1H-pyrrolo[2,3-b]pyridine-6,3-diyl))bis(methylene))bis(azanediyl))bis(methylene))bis(pyrrolidin-2-one);
(S)-5-((((6-(2,2′-dichloro-3′-(2-((S)-2-hydroxypropyl)-8-methoxy-1,2,3,4-tetrahydroisoquinolin-6-yl)-[1,1′-biphenyl]-3-yl)-2-methoxypyridin-3-yl)methyl)amino)methyl)pyrrolidin-2-one;
(R)-5-((((6-(2,2′-dichloro-3′-(2-((S)-2-hydroxypropyl)-8-methoxy-1,2,3,4-tetrahydroisoquinolin-6-yl)-[1,1′-biphenyl]-3-yl)-2-methoxypyridin-3-yl)methyl)amino)methyl)pyrrolidin-2-one;
(S)-5-((((6-(2,2′-dichloro-3′-(2-((R)-2-hydroxypropyl)-8-methoxy-1,2,3,4-tetrahydroisoquinolin-6-yl)-[1,1′-biphenyl]-3-yl)-2-methoxypyridin-3-yl)methyl)amino)methyl)pyrrolidin-2-one;
(R)-5-((((6-(2,2′-dichloro-3′-(2-((R)-2-hydroxypropyl)-8-methoxy-1,2,3,4-tetrahydroisoquinolin-6-yl)-[1,1′-biphenyl]-3-yl)-2-methoxypyridin-3-yl)methyl)amino)methyl)pyrrolidin-2-one;
(S)-5-((((6-(2,2′-dichloro-3′-(2-((R)-2-hydroxypropyl)-8-methoxy-1,2,3,4-tetrahydroisoquinolin-6-yl)-[1,1′-biphenyl]-3-yl)-2-methoxypyridin-3-yl)methyl)amino)methyl)pyrrolidin-2-one;
(R)-5-((((6-(2,2′-dichloro-3′-(2-((S)-2-hydroxypropyl)-8-methoxy-1,2,3,4-tetrahydroisoquinolin-6-yl)-[1,1′-biphenyl]-3-yl)-2-methoxypyridin-3-yl)methyl)amino)methyl)pyrrolidin-2-one;
(S)-5-((((6-(2,2′-dichloro-3′-(2-((S)-2-hydroxypropyl)-8-methoxy-1,2,3,4-tetrahydroisoquinolin-6-yl)-[1,1′-biphenyl]-3-yl)-2-methoxypyridin-3-yl)methyl)amino)methyl)pyrrolidin-2-one;
(R)-5-((((6-(2,2′-dichloro-3′-(2-((R)-2-hydroxypropyl)-8-methoxy-1,2,3,4-tetrahydroisoquinolin-6-yl)-[1,1′-biphenyl]-3-yl)-2-methoxypyridin-3-yl)methyl)amino)methyl)pyrrolidin-2-one;
(S)-5-((((6-(2,2′-dichloro-3′-(2-methyl-3-(((((S)-5-oxopyrrolidin-2-yl)methyl)amino)methyl)pyrazolo[1,5-a]pyrimidin-6-yl)-[1,1′-biphenyl]-3-yl)-2-methoxypyridin-3-yl)methyl)amino)methyl)pyrrolidin-2-one;
(R)-5-((((6-(2,2′-dichloro-3′-(2-methyl-3-(((((S)-5-oxopyrrolidin-2-yl)methyl)amino)methyl)pyrazolo[1,5-a]pyrimidin-6-yl)-[1,1′-biphenyl]-3-yl)-2-methoxypyridin-3-yl)methyl)amino)methyl)pyrrolidin-2-one;
(S)-5-((((6-(2,2′-dichloro-3′-(2-methyl-3-(((((S)-5-oxopyrrolidin-2-yl)methyl)amino)methyl)pyrazolo[1,5-a]pyrimidin-6-yl)-[1,1′-biphenyl]-3-yl)-2-methoxypyridin-3-yl)methyl)amino)methyl)pyrrolidin-2-one;
(R)-5-((((6-(2,2′-dichloro-3′-(2-methyl-3-(((((R)-5-oxopyrrolidin-2-yl)methyl)amino)methyl)pyrazolo[1,5-a]pyrimidin-6-yl)-[1,1′-biphenyl]-3-yl)-2-methoxypyridin-3-yl)methyl)amino)methyl)pyrrolidin-2-one;
7-(2,2′-dichloro-3′-(6-methoxy-5-(((((S)-5-oxopyrrolidin-2-yl)methyl)amino)methyl)pyridin-2-yl)-[1,1′-biphenyl]-3-yl)-2-(((((S)-5-oxopyrrolidin-2-yl)methyl)amino)methyl)quinazolin-4(1H)-one;
7-(2,2′-dichloro-3′-(6-methoxy-5-(((((R)-5-oxopyrrolidin-2-yl)methyl)amino)methyl)pyridin-2-yl)-[1,1′-biphenyl]-3-yl)-2-(((((S)-5-oxopyrrolidin-2-yl)methyl)amino)methyl)quinazolin-4(1H)-one;
7-(2,2′-dichloro-3′-(6-methoxy-5-(((((S)-5-oxopyrrolidin-2-yl)methyl)amino)methyl)pyridin-2-yl)-[1,1′-biphenyl]-3-yl)-2-(((((R)-5-oxopyrrolidin-2-yl)methyl)amino)methyl)quinazolin-4(1H)-one;
7-(2,2′-dichloro-3′-(6-methoxy-5-(((((R)-5-oxopyrrolidin-2-yl)methyl)amino)methyl)pyridin-2-yl)-[1,1′-biphenyl]-3-yl)-2-(((((R)-5-oxopyrrolidin-2-yl)methyl)amino)methyl)quinazolin-4(1H)-one;
(S)-5-((((6-(2,2′-dichloro-3′-(2-(((((S)-5-oxopyrrolidin-2-yl)methyl)amino)methyl)quinazolin-6-yl)-[1,1′-biphenyl]-3-yl)-2-methoxypyridin-3-yl)methyl)amino)methyl)pyrrolidin-2-one;
(R)-5-((((6-(2,2′-dichloro-3′-(2-(((((S)-5-oxopyrrolidin-2-yl)methyl)amino)methyl)quinazolin-6-yl)-[1,1′-biphenyl]-3-yl)-2-methoxypyridin-3-yl)methyl)amino)methyl)pyrrolidin-2-one;
(S)-5-((((6-(2,2′-dichloro-3′-(2-(((((R)-5-oxopyrrolidin-2-yl)methyl)amino)methyl)quinazolin-6-yl)-[1,1′-biphenyl]-3-yl)-2-methoxypyridin-3-yl)methyl)amino)methyl)pyrrolidin-2-one;
(R)-5-((((6-(2,2′-dichloro-3′-(2-(((((R)-5-oxopyrrolidin-2-yl)methyl)amino)methyl)quinazolin-6-yl)-[1,1′-biphenyl]-3-yl)-2-methoxypyridin-3-yl)methyl)amino)methyl)pyrrolidin-2-one;
tert-butyl (S)-((6-(2,2′-dichloro-3′-(2-(hydroxymethyl)-3-methyl-4-oxo-3,4-dihydropyrrolo[2,1-f][1,2,4]triazin-6-yl)-[1,1′-biphenyl]-3-yl)-2-methoxypyridin-3-yl)methyl)((5-oxopyrrolidin-2-yl)methyl)carbamate;
tert-butyl (r)-((6-(2,2′-dichloro-3′-(2-(hydroxymethyl)-3-methyl-4-oxo-3,4-dihydropyrrolo[2,1-f][1,2,4]triazin-6-yl)-[1,1′-biphenyl]-3-yl)-2-methoxypyridin-3-yl)methyl)((5-oxopyrrolidin-2-yl)methyl)carbamate;
(S)-5-((((6-(2,2′-dichloro-3′-(5-methoxy-2-(((S)-5-oxopyrrolidin-2-yl)methyl)-1,2,3,4-tetrahydroisoquinolin-7-yl)-[1,1′-biphenyl]-3-yl)-2-methoxypyridin-3-yl)methyl)amino)methyl)pyrrolidin-2-one;
(R)-5-((((6-(2,2′-dichloro-3′-(5-methoxy-2-(((S)-5-oxopyrrolidin-2-yl)methyl)-1,2,3,4-tetrahydroisoquinolin-7-yl)-[1,1′-biphenyl]-3-yl)-2-methoxypyridin-3-yl)methyl)amino)methyl)pyrrolidin-2-one;
(S)-5-((((6-(2,2′-dichloro-3′-(5-methoxy-2-(((R)-5-oxopyrrolidin-2-yl)methyl)-1,2,3,4-tetrahydroisoquinolin-7-yl)-[1,1′-biphenyl]-3-yl)-2-methoxypyridin-3-yl)methyl)amino)methyl)pyrrolidin-2-one;
(R)-5-((((6-(2,2′-dichloro-3′-(5-methoxy-2-(((R)-5-oxopyrrolidin-2-yl)methyl)-1,2,3,4-tetrahydroisoquinolin-7-yl)-[1,1′-biphenyl]-3-yl)-2-methoxypyridin-3-yl)methyl)amino)methyl)pyrrolidin-2-one;
(S)-5-((6-(2,2′-dichloro-3′-(6-methoxy-5-(((((S)-5-oxopyrrolidin-2-yl)methyl)amino)methyl)pyridin-2-yl)-[1,1′-biphenyl]-3-yl)-8-fluoro-3,4-dihydroisoquinolin-2(1H)-yl)methyl)pyrrolidin-2-one;
(R)-5-((6-(2,2′-dichloro-3′-(6-methoxy-5-(((((S)-5-oxopyrrolidin-2-yl)methyl)amino)methyl)pyridin-2-yl)-[1,1′-biphenyl]-3-yl)-8-fluoro-3,4-dihydroisoquinolin-2(1H)-yl)methyl)pyrrolidin-2-one;
(S)-5-((6-(2,2′-dichloro-3′-(6-methoxy-5-(((((R)-5-oxopyrrolidin-2-yl)methyl)amino)methyl)pyridin-2-yl)-[1,1′-biphenyl]-3-yl)-8-fluoro-3,4-dihydroisoquinolin-2(1H)-yl)methyl)pyrrolidin-2-one;
(R)-5-((6-(2,2′-dichloro-3′-(6-methoxy-5-(((((R)-5-oxopyrrolidin-2-yl)methyl)amino)methyl)pyridin-2-yl)-[1,1′-biphenyl]-3-yl)-8-fluoro-3,4-dihydroisoquinolin-2(1H)-yl)methyl)pyrrolidin-2-one;
(S)-5-((((6-(2,2′-dichloro-3′-(6-(((S)-5-oxopyrrolidin-2-yl)methyl)-5,6,7,8-tetrahydro-1,6-naphthyridin-2-yl)-[1,1′-biphenyl]-3-yl)-2-methoxypyridin-3-yl)methyl)amino)methyl)pyrrolidin-2-one;
(R)-5-((((6-(2,2′-dichloro-3′-(6-(((S)-5-oxopyrrolidin-2-yl)methyl)-5,6,7,8-tetrahydro-1,6-naphthyridin-2-yl)-[1,1′-biphenyl]-3-yl)-2-methoxypyridin-3-yl)methyl)amino)methyl)pyrrolidin-2-one;
(S)-5-((((6-(2,2′-dichloro-3′-(6-(((R)-5-oxopyrrolidin-2-yl)methyl)-5,6,7,8-tetrahydro-1,6-naphthyridin-2-yl)-[1,1′-biphenyl]-3-yl)-2-methoxypyridin-3-yl)methyl)amino)methyl)pyrrolidin-2-one;
(R)-5-((((6-(2,2′-dichloro-3′-(6-(((R)-5-oxopyrrolidin-2-yl)methyl)-5,6,7,8-tetrahydro-1,6-naphthyridin-2-yl)-[1,1′-biphenyl]-3-yl)-2-methoxypyridin-3-yl)methyl)amino)methyl)pyrrolidin-2-one;
(S)-5-((8-chloro-7-(2,2′-dichloro-3′-(6-methoxy-5-(((((S)-5-oxopyrrolidin-2-yl)methyl)amino)methyl)pyridin-2-yl)-[1,1′-biphenyl]-3-yl)-3,4-dihydroisoquinolin-2(1H)-yl)methyl)pyrrolidin-2-one;
(R)-5-((8-chloro-7-(2,2′-dichloro-3′-(6-methoxy-5-(((((S)-5-oxopyrrolidin-2-yl)methyl)amino)methyl)pyridin-2-yl)-[1,1′-biphenyl]-3-yl)-3,4-dihydroisoquinolin-2(1H)-yl)methyl)pyrrolidin-2-one;
(S)-5-((8-chloro-7-(2,2′-dichloro-3′-(6-methoxy-5-(((((R)-5-oxopyrrolidin-2-yl)methyl)amino)methyl)pyridin-2-yl)-[1,1′-biphenyl]-3-yl)-3,4-dihydroisoquinolin-2(1H)-yl)methyl)pyrrolidin-2-one;
(R)-5-((8-chloro-7-(2,2′-dichloro-3′-(6-methoxy-5-(((((R)-5-oxopyrrolidin-2-yl)methyl)amino)methyl)pyridin-2-yl)-[1,1′-biphenyl]-3-yl)-3,4-dihydroisoquinolin-2(1H)-yl)methyl)pyrrolidin-2-one;
(S)-5-((7-(2,2′-dichloro-3′-(6-methoxy-5-(((((S)-5-oxopyrrolidin-2-yl)methyl)amino)methyl)pyridin-2-yl)-[1,1′-biphenyl]-3-yl)-8-fluoro-3,4-dihydroisoquinolin-2(1H)-yl)methyl)pyrrolidin-2-one;
(R)-5-((7-(2,2′-dichloro-3′-(6-methoxy-5-(((((S)-5-oxopyrrolidin-2-yl)methyl)amino)methyl)pyridin-2-yl)-[1,1′-biphenyl]-3-yl)-8-fluoro-3,4-dihydroisoquinolin-2(1H)-yl)methyl)pyrrolidin-2-one;
(S)-5-((7-(2,2′-dichloro-3′-(6-methoxy-5-(((((R)-5-oxopyrrolidin-2-yl)methyl)amino)methyl)pyridin-2-yl)-[1,1′-biphenyl]-3-yl)-8-fluoro-3,4-dihydroisoquinolin-2(1H)-yl)methyl)pyrrolidin-2-one;
(R)-5-((7-(2,2′-dichloro-3′-(6-methoxy-5-(((((R)-5-oxopyrrolidin-2-yl)methyl)amino)methyl)pyridin-2-yl)-[1,1′-biphenyl]-3-yl)-8-fluoro-3,4-dihydroisoquinolin-2(1H)-yl)methyl)pyrrolidin-2-one;
8-(2,2′-dichloro-3′-(3-chloro-2-(((((S)-5-oxopyrrolidin-2-yl)methyl)amino)methyl)imidazo[1,2-a]pyridin-7-yl)-[1,1′-biphenyl]-3-yl)-3-(((((S)-5-oxopyrrolidin-2-yl)methyl)amino)methyl)-4H-pyrido[1,2-a]pyrimidin-4-one;
8-(2,2′-dichloro-3′-(3-chloro-2-(((((R)-5-oxopyrrolidin-2-yl)methyl)amino)methyl)imidazo[1,2-a]pyridin-7-yl)-[1,1′-biphenyl]-3-yl)-3-(((((S)-5-oxopyrrolidin-2-yl)methyl)amino)methyl)-4H-pyrido[1,2-a]pyrimidin-4-one;
8-(2,2′-dichloro-3′-(3-chloro-2-(((((S)-5-oxopyrrolidin-2-yl)methyl)amino)methyl)imidazo[1,2-a]pyridin-7-yl)-[1,1′-biphenyl]-3-yl)-3-(((((R)-5-oxopyrrolidin-2-yl)methyl)amino)methyl)-4H-pyrido[1,2-a]pyrimidin-4-one;
8-(2,2′-dichloro-3′-(3-chloro-2-(((((R)-5-oxopyrrolidin-2-yl)methyl)amino)methyl)imidazo[1,2-a]pyridin-7-yl)-[1,1′-biphenyl]-3-yl)-3-(((((R)-5-oxopyrrolidin-2-yl)methyl)amino)methyl)-4H-pyrido[1,2-a]pyrimidin-4-one;
(5S,5'S)-5,5′-(((((2,2′-dichloro-[1,1′-biphenyl]-3,3′-diyl)bis(3-chloroimidazo[1,2-a]pyridine-7,2-diyl))bis(methylene))bis(azanediyl))bis(methylene))bis(pyrrolidin-2-one);
(5R,5'S)-5,5′-(((((2,2′-dichloro-[1,1′-biphenyl]-3,3′-diyl)bis(3-chloroimidazo[1,2-a]pyridine-7,2-diyl))bis(methylene))bis(azanediyl))bis(methylene))bis(pyrrolidin-2-one);
(5S,5′R)-5,5′-(((((2,2′-dichloro-[1,1′-biphenyl]-3,3′-diyl)bis(3-chloroimidazo[1,2-a]pyridine-7,2-diyl))bis(methylene))bis(azanediyl))bis(methylene))bis(pyrrolidin-2-one);
(5R,5′R)-5,5′-(((((2,2′-dichloro-[1,1′-biphenyl]-3,3′-diyl)bis(3-chloroimidazo[1,2-a]pyridine-7,2-diyl))bis(methylene))bis(azanediyl))bis(methylene))bis(pyrrolidin-2-one);
8-(2,2′-dichloro-3′-(6-chloro-4-methoxy-5-(((((S)-5-oxopyrrolidin-2-yl)methyl)amino)methyl)pyridin-2-yl)-[1,1′-biphenyl]-3-yl)-3-(((((S)-5-oxopyrrolidin-2-yl)methyl)amino)methyl)-4H-pyrido[1,2-a]pyrimidin-4-one;
8-(2,2′-dichloro-3′-(6-chloro-4-methoxy-5-(((((R)-5-oxopyrrolidin-2-yl)methyl)amino)methyl)pyridin-2-yl)-[1,1′-biphenyl]-3-yl)-3-(((((S)-5-oxopyrrolidin-2-yl)methyl)amino)methyl)-4H-pyrido[1,2-a]pyrimidin-4-one;
8-(2,2′-dichloro-3′-(6-chloro-4-methoxy-5-(((((S)-5-oxopyrrolidin-2-yl)methyl)amino)methyl)pyridin-2-yl)-[1,1′-biphenyl]-3-yl)-3-(((((R)-5-oxopyrrolidin-2-yl)methyl)amino)methyl)-4H-pyrido[1,2-a]pyrimidin-4-one;
8-(2,2′-dichloro-3′-(6-chloro-4-methoxy-5-(((((R)-5-oxopyrrolidin-2-yl)methyl)amino)methyl)pyridin-2-yl)-[1,1′-biphenyl]-3-yl)-3-(((((R)-5-oxopyrrolidin-2-yl)methyl)amino)methyl)-4H-pyrido[1,2-a]pyrimidin-4-one;
(S)-5-((((6-(2,2′-dichloro-3′-((S)-8-((((S)-5-oxopyrrolidin-2-yl)methyl)amino)-5,6,7,8-tetrahydroquinolin-3-yl)-[1,1′-biphenyl]-3-yl)-2-methoxypyridin-3-yl)methyl)amino)methyl)pyrrolidin-2-one;
(S)-5-((((6-(2,2′-dichloro-3′-((R)-8-((((S)-5-oxopyrrolidin-2-yl)methyl)amino)-5,6,7,8-tetrahydroquinolin-3-yl)-[1,1′-biphenyl]-3-yl)-2-methoxypyridin-3-yl)methyl)amino)methyl)pyrrolidin-2-one;
(S)-5-((((6-(2,2′-dichloro-3′-((S)-8-((((R)-5-oxopyrrolidin-2-yl)methyl)amino)-5,6,7,8-tetrahydroquinolin-3-yl)-[1,1′-biphenyl]-3-yl)-2-methoxypyridin-3-yl)methyl)amino)methyl)pyrrolidin-2-one;
(S)-5-((((6-(2,2′-dichloro-3′-((R)-8-((((R)-5-oxopyrrolidin-2-yl)methyl)amino)-5,6,7,8-tetrahydroquinolin-3-yl)-[1,1′-biphenyl]-3-yl)-2-methoxypyridin-3-yl)methyl)amino)methyl)pyrrolidin-2-one;
(R)-5-((((6-(2,2′-dichloro-3′-((S)-8-((((S)-5-oxopyrrolidin-2-yl)methyl)amino)-5,6,7,8-tetrahydroquinolin-3-yl)-[1,1′-biphenyl]-3-yl)-2-methoxypyridin-3-yl)methyl)amino)methyl)pyrrolidin-2-one;
(R)-5-((((6-(2,2′-dichloro-3′-((R)-8-((((S)-5-oxopyrrolidin-2-yl)methyl)amino)-5,6,7,8-tetrahydroquinolin-3-yl)-[1,1′-biphenyl]-3-yl)-2-methoxypyridin-3-yl)methyl)amino)methyl)pyrrolidin-2-one;
(R)-5-((((6-(2,2′-dichloro-3′-((S)-8-((((R)-5-oxopyrrolidin-2-yl)methyl)amino)-5,6,7,8-tetrahydroquinolin-3-yl)-[1,1′-biphenyl]-3-yl)-2-methoxypyridin-3-yl)methyl)amino)methyl)pyrrolidin-2-one;
(R)-5-((((6-(2,2′-dichloro-3′-((R)-8-((((R)-5-oxopyrrolidin-2-yl)methyl)amino)-5,6,7,8-tetrahydroquinolin-3-yl)-[1,1′-biphenyl]-3-yl)-2-methoxypyridin-3-yl)methyl)amino)methyl)pyrrolidin-2-one;
6-(2,2′-dichloro-3′-(6-methoxy-5-(((((S)-5-oxopyrrolidin-2-yl)methyl)amino)methyl)pyridin-2-yl)-[1,1′-biphenyl]-3-yl)-2-(((((S)-5-oxopyrrolidin-2-yl)methyl)amino)methyl)quinazolin-4(1H)-one;
6-(2,2′-dichloro-3′-(6-methoxy-5-(((((R)-5-oxopyrrolidin-2-yl)methyl)amino)methyl)pyridin-2-yl)-[1,1′-biphenyl]-3-yl)-2-(((((S)-5-oxopyrrolidin-2-yl)methyl)amino)methyl)quinazolin-4(1H)-one;
6-(2,2′-dichloro-3′-(6-methoxy-5-(((((S)-5-oxopyrrolidin-2-yl)methyl)amino)methyl)pyridin-2-yl)-[1,1′-biphenyl]-3-yl)-2-(((((R)-5-oxopyrrolidin-2-yl)methyl)amino)methyl)quinazolin-4(1H)-one;
6-(2,2′-dichloro-3′-(6-methoxy-5-(((((R)-5-oxopyrrolidin-2-yl)methyl)amino)methyl)pyridin-2-yl)-[1,1′-biphenyl]-3-yl)-2-(((((R)-5-oxopyrrolidin-2-yl)methyl)amino)methyl)quinazolin-4(1H)-one;
(S)-5-((((6-(2,2′-dichloro-3′-(3-(((((S)-5-oxopyrrolidin-2-yl)methyl)amino)methyl)-1H-pyrazolo[3,4-b]pyridin-5-yl)-[1,1′-biphenyl]-3-yl)-2-methoxypyridin-3-yl)methyl)amino)methyl)pyrrolidin-2-one;
(R)-5-((((6-(2,2′-dichloro-3′-(3-(((((S)-5-oxopyrrolidin-2-yl)methyl)amino)methyl)-1H-pyrazolo[3,4-b]pyridin-5-yl)-[1,1′-biphenyl]-3-yl)-2-methoxypyridin-3-yl)methyl)amino)methyl)pyrrolidin-2-one;
(S)-5-((((6-(2,2′-dichloro-3′-(3-(((((R)-5-oxopyrrolidin-2-yl)methyl)amino)methyl)-1H-pyrazolo[3,4-b]pyridin-5-yl)-[1,1′-biphenyl]-3-yl)-2-methoxypyridin-3-yl)methyl)amino)methyl)pyrrolidin-2-one;
(R)-5-((((6-(2,2′-dichloro-3′-(3-(((((R)-5-oxopyrrolidin-2-yl)methyl)amino)methyl)-1H-pyrazolo[3,4-b]pyridin-5-yl)-[1,1′-biphenyl]-3-yl)-2-methoxypyridin-3-yl)methyl)amino)methyl)pyrrolidin-2-one;
(S)-5-((((6-(2,2′-dichloro-3′-((S)-5-((((S)-5-oxopyrrolidin-2-yl)methyl)amino)-5,6,7,8-tetrahydroquinolin-2-yl)-[1,1′-biphenyl]-3-yl)-2-methoxypyridin-3-yl)methyl)amino)methyl)pyrrolidin-2-one;
(S)-5-((((6-(2,2′-dichloro-3′-((R)-5-((((S)-5-oxopyrrolidin-2-yl)methyl)amino)-5,6,7,8-tetrahydroquinolin-2-yl)-[1,1′-biphenyl]-3-yl)-2-methoxypyridin-3-yl)methyl)amino)methyl)pyrrolidin-2-one;
(S)-5-((((6-(2,2′-dichloro-3′-((S)-5-((((R)-5-oxopyrrolidin-2-yl)methyl)amino)-5,6,7,8-tetrahydroquinolin-2-yl)-[1,1′-biphenyl]-3-yl)-2-methoxypyridin-3-yl)methyl)amino)methyl)pyrrolidin-2-one;
(S)-5-((((6-(2,2′-dichloro-3′-((R)-5-((((R)-5-oxopyrrolidin-2-yl)methyl)amino)-5,6,7,8-tetrahydroquinolin-2-yl)-[1,1′-biphenyl]-3-yl)-2-methoxypyridin-3-yl)methyl)amino)methyl)pyrrolidin-2-one;
(R)-5-((((6-(2,2′-dichloro-3′-((S)-5-((((S)-5-oxopyrrolidin-2-yl)methyl)amino)-5,6,7,8-tetrahydroquinolin-2-yl)-[1,1′-biphenyl]-3-yl)-2-methoxypyridin-3-yl)methyl)amino)methyl)pyrrolidin-2-one;
(R)-5-((((6-(2,2′-dichloro-3′-((R)-5-((((S)-5-oxopyrrolidin-2-yl)methyl)amino)-5,6,7,8-tetrahydroquinolin-2-yl)-[1,1′-biphenyl]-3-yl)-2-methoxypyridin-3-yl)methyl)amino)methyl)pyrrolidin-2-one;
(R)-5-((((6-(2,2′-dichloro-3′-((S)-5-((((R)-5-oxopyrrolidin-2-yl)methyl)amino)-5,6,7,8-tetrahydroquinolin-2-yl)-[1,1′-biphenyl]-3-yl)-2-methoxypyridin-3-yl)methyl)amino)methyl)pyrrolidin-2-one;
(R)-5-((((6-(2,2′-dichloro-3′-((R)-5-((((R)-5-oxopyrrolidin-2-yl)methyl)amino)-5,6,7,8-tetrahydroquinolin-2-yl)-[1,1′-biphenyl]-3-yl)-2-methoxypyridin-3-yl)methyl)amino)methyl)pyrrolidin-2-one;
6-(2,2′-dichloro-3′-(6-methoxy-5-(((((S)-5-oxopyrrolidin-2-yl)methyl)amino)methyl)pyridin-2-yl)-[1,1′-biphenyl]-3-yl)-2-((((S)-2-hydroxypropyl)(methyl)amino)methyl)-3-methylpyrrolo[2,1-f][1,2,4]triazin-4(3H)-one;
6-(2,2′-dichloro-3′-(6-methoxy-5-(((((R)-5-oxopyrrolidin-2-yl)methyl)amino)methyl)pyridin-2-yl)-[1,1′-biphenyl]-3-yl)-2-((((S)-2-hydroxypropyl)(methyl)amino)methyl)-3-methylpyrrolo[2,1-f][1,2,4]triazin-4(3H)-one;
6-(2,2′-dichloro-3′-(6-methoxy-5-(((((S)-5-oxopyrrolidin-2-yl)methyl)amino)methyl)pyridin-2-yl)-[1,1′-biphenyl]-3-yl)-2-((((R)-2-hydroxypropyl)(methyl)amino)methyl)-3-methylpyrrolo[2,1-f][1,2,4]triazin-4(3H)-one;
6-(2,2′-dichloro-3′-(6-methoxy-5-(((((R)-5-oxopyrrolidin-2-yl)methyl)amino)methyl)pyridin-2-yl)-[1,1′-biphenyl]-3-yl)-2-((((R)-2-hydroxypropyl)(methyl)amino)methyl)-3-methylpyrrolo[2,1-f][1,2,4]triazin-4(3H)-one;
(S)-6-(2,2′-dichloro-3′-(6-methoxy-5-((((5-oxopyrrolidin-2-yl)methyl)amino)methyl)pyridin-2-yl)-[1,1′-biphenyl]-3-yl)-2-(((3-fluoropropyl)amino)methyl)-3-methylpyrrolo[2,1-f][1,2,4]triazin-4(3H)-one;
(R)-6-(2,2′-dichloro-3′-(6-methoxy-5-((((5-oxopyrrolidin-2-yl)methyl)amino)methyl)pyridin-2-yl)-[1,1′-biphenyl]-3-yl)-2-(((3-fluoropropyl)amino)methyl)-3-methylpyrrolo[2,1-f][1,2,4]triazin-4(3H)-one;
(S)—N-(2-aminoethyl)-6-(2,2′-dichloro-3′-(6-methoxy-5-((((5-oxopyrrolidin-2-yl)methyl)amino)methyl)pyridin-2-yl)-[1,1′-biphenyl]-3-yl)imidazo[1,2-b]pyridazine-2-carboxamide;
(R)—N-(2-aminoethyl)-6-(2,2′-dichloro-3′-(6-methoxy-5-((((5-oxopyrrolidin-2-yl)methyl)amino)methyl)pyridin-2-yl)-[1,1′-biphenyl]-3-yl)imidazo[1,2-b]pyridazine-2-carboxamide;
N—((S)-2-aminopropyl)-6-(2,2′-dichloro-3′-(6-methoxy-5-(((((S)-5-oxopyrrolidin-2-yl)methyl)amino)methyl)pyridin-2-yl)-[1,1′-biphenyl]-3-yl)imidazo[1,2-b]pyridazine-2-carboxamide;
N—((R)-2-aminopropyl)-6-(2,2′-dichloro-3′-(6-methoxy-5-(((((S)-5-oxopyrrolidin-2-yl)methyl)amino)methyl)pyridin-2-yl)-[1,1′-biphenyl]-3-yl)imidazo[1,2-b]pyridazine-2-carboxamide;
N—((S)-2-aminopropyl)-6-(2,2′-dichloro-3′-(6-methoxy-5-(((((R)-5-oxopyrrolidin-2-yl)methyl)amino)methyl)pyridin-2-yl)-[1,1′-biphenyl]-3-yl)imidazo[1,2-b]pyridazine-2-carboxamide;
N—((R)-2-aminopropyl)-6-(2,2′-dichloro-3′-(6-methoxy-5-(((((R)-5-oxopyrrolidin-2-yl)methyl)amino)methyl)pyridin-2-yl)-[1,1′-biphenyl]-3-yl)imidazo[1,2-b]pyridazine-2-carboxamide;
(S)—N-(2-(1H-imidazol-4-yl)ethyl)-6-(2,2′-dichloro-3′-(6-methoxy-5-((((5-oxopyrrolidin-2-yl)methyl)amino)methyl)pyridin-2-yl)-[1,1′-biphenyl]-3-yl)imidazo[1,2-b]pyridazine-2-carboxamide;
(R)—N-(2-(1H-imidazol-4-yl)ethyl)-6-(2,2′-dichloro-3′-(6-methoxy-5-((((5-oxopyrrolidin-2-yl)methyl)amino)methyl)pyridin-2-yl)-[1,1′-biphenyl]-3-yl)imidazo[1,2-b]pyridazine-2-carboxamide;
8-(2,2′-dichloro-3′-(2-(((((S)-5-oxopyrrolidin-2-yl)methyl)amino)methyl)thiazol-5-yl)-[1,1′-biphenyl]-3-yl)-3-(((((S)-5-oxopyrrolidin-2-yl)methyl)amino)methyl)-4H-pyrido[1,2-a]pyrimidin-4-one;
8-(2,2′-dichloro-3′-(2-(((((R)-5-oxopyrrolidin-2-yl)methyl)amino)methyl)thiazol-5-yl)-[1,1′-biphenyl]-3-yl)-3-(((((S)-5-oxopyrrolidin-2-yl)methyl)amino)methyl)-4H-pyrido[1,2-a]pyrimidin-4-one;
8-(2,2′-dichloro-3′-(2-(((((S)-5-oxopyrrolidin-2-yl)methyl)amino)methyl)thiazol-5-yl)-[1,1′-biphenyl]-3-yl)-3-(((((R)-5-oxopyrrolidin-2-yl)methyl)amino)methyl)-4H-pyrido[1,2-a]pyrimidin-4-one;
8-(2,2′-dichloro-3′-(2-(((((R)-5-oxopyrrolidin-2-yl)methyl)amino)methyl)thiazol-5-yl)-[1,1′-biphenyl]-3-yl)-3-(((((R)-5-oxopyrrolidin-2-yl)methyl)amino)methyl)-4H-pyrido[1,2-a]pyrimidin-4-one;
8-(2,2′-dichloro-3′-(2-(((((S)-5-oxopyrrolidin-2-yl)methyl)amino)methyl)thiazol-4-yl)-[1,1′-biphenyl]-3-yl)-3-(((((S)-5-oxopyrrolidin-2-yl)methyl)amino)methyl)-4H-pyrido[1,2-a]pyrimidin-4-one;
8-(2,2′-dichloro-3′-(2-(((((R)-5-oxopyrrolidin-2-yl)methyl)amino)methyl)thiazol-4-yl)-[1,1′-biphenyl]-3-yl)-3-(((((S)-5-oxopyrrolidin-2-yl)methyl)amino)methyl)-4H-pyrido[1,2-a]pyrimidin-4-one;
8-(2,2′-dichloro-3′-(2-(((((S)-5-oxopyrrolidin-2-yl)methyl)amino)methyl)thiazol-4-yl)-[1,1′-biphenyl]-3-yl)-3-(((((R)-5-oxopyrrolidin-2-yl)methyl)amino)methyl)-4H-pyrido[1,2-a]pyrimidin-4-one;
8-(2,2′-dichloro-3′-(2-(((((R)-5-oxopyrrolidin-2-yl)methyl)amino)methyl)thiazol-4-yl)-[1,1′-biphenyl]-3-yl)-3-(((((R)-5-oxopyrrolidin-2-yl)methyl)amino)methyl)-4H-pyrido[1,2-a]pyrimidin-4-one;
(S)-5-((((6-(2,2′-dichloro-3′-(6-(((S)-5-oxopyrrolidin-2-yl)methyl)-5,6,7,8-tetrahydropyrido[4,3-d]pyrimidin-2-yl)-[1,1′-biphenyl]-3-yl)-2-methoxypyridin-3-yl)methyl)amino)methyl)pyrrolidin-2-one;
(R)-5-((((6-(2,2′-dichloro-3′-(6-(((S)-5-oxopyrrolidin-2-yl)methyl)-5,6,7,8-tetrahydropyrido[4,3-d]pyrimidin-2-yl)-[1,1′-biphenyl]-3-yl)-2-methoxypyridin-3-yl)methyl)amino)methyl)pyrrolidin-2-one;
(S)-5-((((6-(2,2′-dichloro-3′-(6-(((R)-5-oxopyrrolidin-2-yl)methyl)-5,6,7,8-tetrahydropyrido[4,3-d]pyrimidin-2-yl)-[1,1′-biphenyl]-3-yl)-2-methoxypyridin-3-yl)methyl)amino)methyl)pyrrolidin-2-one;
(R)-5-((((6-(2,2′-dichloro-3′-(6-(((R)-5-oxopyrrolidin-2-yl)methyl)-5,6,7,8-tetrahydropyrido[4,3-d]pyrimidin-2-yl)-[1,1′-biphenyl]-3-yl)-2-methoxypyridin-3-yl)methyl)amino)methyl)pyrrolidin-2-one;
(S)-5-((((6-(2,2′-dichloro-3′-(1-methyl-3-(((((S)-tetrahydrofuran-2-yl)methyl)amino)methyl)-1H-pyrrolo[2,3-b]pyridin-6-yl)-[1,1′-biphenyl]-3-yl)-2-methoxypyridin-3-yl)methyl)amino)methyl)pyrrolidin-2-one;
(R)-5-((((6-(2,2′-dichloro-3′-(1-methyl-3-(((((S)-tetrahydrofuran-2-yl)methyl)amino)methyl)-1H-pyrrolo[2,3-b]pyridin-6-yl)-[1,1′-biphenyl]-3-yl)-2-methoxypyridin-3-yl)methyl)amino)methyl)pyrrolidin-2-one;
(S)-5-((((6-(2,2′-dichloro-3′-(1-methyl-3-(((((R)-tetrahydrofuran-2-yl)methyl)amino)methyl)-1H-pyrrolo[2,3-b]pyridin-6-yl)-[1,1′-biphenyl]-3-yl)-2-methoxypyridin-3-yl)methyl)amino)methyl)pyrrolidin-2-one;
(R)-5-((((6-(2,2′-dichloro-3′-(1-methyl-3-(((((R)-tetrahydrofuran-2-yl)methyl)amino)methyl)-1H-pyrrolo[2,3-b]pyridin-6-yl)-[1,1′-biphenyl]-3-yl)-2-methoxypyridin-3-yl)methyl)amino)methyl)pyrrolidin-2-one;
(S)-5-((((6-(2,2′-dichloro-3′-(3-((((S)-2-methoxypropyl)amino)methyl)-1-methyl-1H-pyrrolo[2,3-b]pyridin-6-yl)-[1,1′-biphenyl]-3-yl)-2-methoxypyridin-3-yl)methyl)amino)methyl)pyrrolidin-2-one;
(S)-5-((((6-(2,2′-dichloro-3′-(3-((((R)-2-methoxypropyl)amino)methyl)-1-methyl-1H-pyrrolo[2,3-b]pyridin-6-yl)-[1,1′-biphenyl]-3-yl)-2-methoxypyridin-3-yl)methyl)amino)methyl)pyrrolidin-2-one;
(R)-5-((((6-(2,2′-dichloro-3′-(3-((((S)-2-methoxypropyl)amino)methyl)-1-methyl-1H-pyrrolo[2,3-b]pyridin-6-yl)-[1,1′-biphenyl]-3-yl)-2-methoxypyridin-3-yl)methyl)amino)methyl)pyrrolidin-2-one;
(R)-5-((((6-(2,2′-dichloro-3′-(3-((((R)-2-methoxypropyl)amino)methyl)-1-methyl-1H-pyrrolo[2,3-b]pyridin-6-yl)-[1,1′-biphenyl]-3-yl)-2-methoxypyridin-3-yl)methyl)amino)methyl)pyrrolidin-2-one;
(S)-2-(((2-oxaspiro[3.3]heptan-6-yl)amino)methyl)-6-(2,2′-dichloro-3′-(6-methoxy-5-((((5-oxopyrrolidin-2-yl)methyl)amino)methyl)pyridin-2-yl)-[1,1′-biphenyl]-3-yl)-3-methylpyrrolo[2,1-f][1,2,4]triazin-4(3H)-one;
(R)-2-(((2-oxaspiro[3.3]heptan-6-yl)amino)methyl)-6-(2,2′-dichloro-3′-(6-methoxy-5-((((5-oxopyrrolidin-2-yl)methyl)amino)methyl)pyridin-2-yl)-[1,1′-biphenyl]-3-yl)-3-methylpyrrolo[2,1-f][1,2,4]triazin-4(3H)-one;
(S)-2-((2-oxa-6-azaspiro[3.3]heptan-6-yl)methyl)-6-(2,2′-dichloro-3′-(6-methoxy-5-((((5-oxopyrrolidin-2-yl)methyl)amino)methyl)pyridin-2-yl)-[1,1′-biphenyl]-3-yl)-3-methylpyrrolo[2,1-f][1,2,4]triazin-4(3H)-one;
(R)-2-((2-oxa-6-azaspiro[3.3]heptan-6-yl)methyl)-6-(2,2′-dichloro-3′-(6-methoxy-5-((((5-oxopyrrolidin-2-yl)methyl)amino)methyl)pyridin-2-yl)-[1,1′-biphenyl]-3-yl)-3-methylpyrrolo[2,1-f][1,2,4]triazin-4(3H)-one;
(S)-6-(2,2′-dichloro-3′-(6-methoxy-5-((((5-oxopyrrolidin-2-yl)methyl)amino)methyl)pyridin-2-yl)-[1,1′-biphenyl]-3-yl)-3-methyl-2-((6-oxo-2,5-diazaspiro[3.4]octan-2-yl)methyl)pyrrolo[2,1-f][1,2,4]triazin-4(3H)-one;
(R)-6-(2,2′-dichloro-3′-(6-methoxy-5-((((5-oxopyrrolidin-2-yl)methyl)amino)methyl)pyridin-2-yl)-[1,1′-biphenyl]-3-yl)-3-methyl-2-((6-oxo-2,5-diazaspiro[3.4]octan-2-yl)methyl)pyrrolo[2,1-f][1,2,4]triazin-4(3H)-one;
6-(2,2′-dichloro-3′-(6-methoxy-5-(((((S)-5-oxopyrrolidin-2-yl)methyl)amino)methyl)pyridin-2-yl)-[1,1′-biphenyl]-3-yl)-3-methyl-2-((((2s,4r)-6-oxo-5-azaspiro[3.4]octan-2-yl)amino)methyl)pyrrolo[2,1-f][1,2,4]triazin-4(3H)-one;
6-(2,2′-dichloro-3′-(6-methoxy-5-(((((S)-5-oxopyrrolidin-2-yl)methyl)amino)methyl)pyridin-2-yl)-[1,1′-biphenyl]-3-yl)-3-methyl-2-((((2r,4s)-6-oxo-5-azaspiro[3.4]octan-2-yl)amino)methyl)pyrrolo[2,1-f][1,2,4]triazin-4(3H)-one;
6-(2,2′-dichloro-3′-(6-methoxy-5-(((((R)-5-oxopyrrolidin-2-yl)methyl)amino)methyl)pyridin-2-yl)-[1,1′-biphenyl]-3-yl)-3-methyl-2-((((2s,4r)-6-oxo-5-azaspiro[3.4]octan-2-yl)amino)methyl)pyrrolo[2,1-f][1,2,4]triazin-4(3H)-one;
6-(2,2′-dichloro-3′-(6-methoxy-5-(((((R)-5-oxopyrrolidin-2-yl)methyl)amino)methyl)pyridin-2-yl)-[1,1′-biphenyl]-3-yl)-3-methyl-2-((((2r,4s)-6-oxo-5-azaspiro[3.4]octan-2-yl)amino)methyl)pyrrolo[2,1-f][1,2,4]triazin-4(3H)-one;
6-(2,2′-dichloro-3′-(6-methoxy-5-(((((S)-5-oxopyrrolidin-2-yl)methyl)amino)methyl)pyridin-2-yl)-[1,1′-biphenyl]-3-yl)-2-(((((R)-4,4-difluoro-5-oxopyrrolidin-2-yl)methyl)amino)methyl)-3-methylpyrrolo[2,1-f][1,2,4]triazin-4(3H)-one;
6-(2,2′-dichloro-3′-(6-methoxy-5-(((((S)-5-oxopyrrolidin-2-yl)methyl)amino)methyl)pyridin-2-yl)-[1,1′-biphenyl]-3-yl)-2-(((((S)-4,4-difluoro-5-oxopyrrolidin-2-yl)methyl)amino)methyl)-3-methylpyrrolo[2,1-f][1,2,4]triazin-4(3H)-one;
6-(2,2′-dichloro-3′-(6-methoxy-5-(((((R)-5-oxopyrrolidin-2-yl)methyl)amino)methyl)pyridin-2-yl)-[1,1′-biphenyl]-3-yl)-2-(((((R)-4,4-difluoro-5-oxopyrrolidin-2-yl)methyl)amino)methyl)-3-methylpyrrolo[2,1-f][1,2,4]triazin-4(3H)-one;
6-(2,2′-dichloro-3′-(6-methoxy-5-(((((R)-5-oxopyrrolidin-2-yl)methyl)amino)methyl)pyridin-2-yl)-[1,1′-biphenyl]-3-yl)-2-(((((S)-4,4-difluoro-5-oxopyrrolidin-2-yl)methyl)amino)methyl)-3-methylpyrrolo[2,1-f][1,2,4]triazin-4(3H)-one;
6-(2,2′-dichloro-3′-(6-methoxy-5-(((((S)-5-oxopyrrolidin-2-yl)methyl)amino)methyl)pyridin-2-yl)-[1,1′-biphenyl]-3-yl)-3-methyl-2-(((2-((R)-5-oxopyrrolidin-2-yl)ethyl)amino)methyl)pyrrolo[2,1-f][1,2,4]triazin-4(3H)-one;
6-(2,2′-dichloro-3′-(6-methoxy-5-(((((S)-5-oxopyrrolidin-2-yl)methyl)amino)methyl)pyridin-2-yl)-[1,1′-biphenyl]-3-yl)-3-methyl-2-(((2-((S)-5-oxopyrrolidin-2-yl)ethyl)amino)methyl)pyrrolo[2,1-f][1,2,4]triazin-4(3H)-one;
6-(2,2′-dichloro-3′-(6-methoxy-5-(((((R)-5-oxopyrrolidin-2-yl)methyl)amino)methyl)pyridin-2-yl)-[1,1′-biphenyl]-3-yl)-3-methyl-2-(((2-((R)-5-oxopyrrolidin-2-yl)ethyl)amino)methyl)pyrrolo[2,1-f][1,2,4]triazin-4(3H)-one;
6-(2,2′-dichloro-3′-(6-methoxy-5-(((((R)-5-oxopyrrolidin-2-yl)methyl)amino)methyl)pyridin-2-yl)-[1,1′-biphenyl]-3-yl)-3-methyl-2-(((2-((S)-5-oxopyrrolidin-2-yl)ethyl)amino)methyl)pyrrolo[2,1-f][1,2,4]triazin-4(3H)-one;
(S)-5-((((6-(2,2′-dichloro-3′-(1-methyl-3-(((((S)-5-oxopyrrolidin-2-yl)methyl)amino)methyl)-1H-indol-6-yl)-[1,1′-biphenyl]-3-yl)-2-methoxypyridin-3-yl)methyl)amino)methyl)pyrrolidin-2-one;
(R)-5-((((6-(2,2′-dichloro-3′-(1-methyl-3-(((((S)-5-oxopyrrolidin-2-yl)methyl)amino)methyl)-1H-indol-6-yl)-[1,1′-biphenyl]-3-yl)-2-methoxypyridin-3-yl)methyl)amino)methyl)pyrrolidin-2-one;
(S)-5-((((6-(2,2′-dichloro-3′-(1-methyl-3-(((((R)-5-oxopyrrolidin-2-yl)methyl)amino)methyl)-1H-indol-6-yl)-[1,1′-biphenyl]-3-yl)-2-methoxypyridin-3-yl)methyl)amino)methyl)pyrrolidin-2-one;
(R)-5-((((6-(2,2′-dichloro-3′-(1-methyl-3-(((((R)-5-oxopyrrolidin-2-yl)methyl)amino)methyl)-1H-indol-6-yl)-[1,1′-biphenyl]-3-yl)-2-methoxypyridin-3-yl)methyl)amino)methyl)pyrrolidin-2-one;
8-(2,2′-dichloro-3′-(1-methyl-3-(((((S)-5-oxopyrrolidin-2-yl)methyl)amino)methyl)-1H-indol-6-yl)-[1,1′-biphenyl]-3-yl)-3-(((((S)-5-oxopyrrolidin-2-yl)methyl)amino)methyl)-4H-pyrido[1,2-a]pyrimidin-4-one;
8-(2,2′-dichloro-3′-(1-methyl-3-(((((R)-5-oxopyrrolidin-2-yl)methyl)amino)methyl)-1H-indol-6-yl)-[1,1′-biphenyl]-3-yl)-3-(((((S)-5-oxopyrrolidin-2-yl)methyl)amino)methyl)-4H-pyrido[1,2-a]pyrimidin-4-one;
8-(2,2′-dichloro-3′-(1-methyl-3-(((((S)-5-oxopyrrolidin-2-yl)methyl)amino)methyl)-1H-indol-6-yl)-[1,1′-biphenyl]-3-yl)-3-(((((R)-5-oxopyrrolidin-2-yl)methyl)amino)methyl)-4H-pyrido[1,2-a]pyrimidin-4-one;
8-(2,2′-dichloro-3′-(1-methyl-3-(((((R)-5-oxopyrrolidin-2-yl)methyl)amino)methyl)-1H-indol-6-yl)-[1,1′-biphenyl]-3-yl)-3-(((((R)-5-oxopyrrolidin-2-yl)methyl)amino)methyl)-4H-pyrido[1,2-a]pyrimidin-4-one;
6-(2,2′-dichloro-3′-(6-methoxy-5-(((((S)-5-oxopyrrolidin-2-yl)methyl)amino)methyl)pyridin-2-yl)-[1,1′-biphenyl]-3-yl)-2-(2-((((S)-5-oxopyrrolidin-2-yl)methyl)amino)ethyl)-2,3-dihydro-1H-pyrrolo[3,4-c]pyridin-1-one;
6-(2,2′-dichloro-3′-(6-methoxy-5-(((((R)-5-oxopyrrolidin-2-yl)methyl)amino)methyl)pyridin-2-yl)-[1,1′-biphenyl]-3-yl)-2-(2-((((S)-5-oxopyrrolidin-2-yl)methyl)amino)ethyl)-2,3-dihydro-1H-pyrrolo[3,4-c]pyridin-1-one;
6-(2,2′-dichloro-3′-(6-methoxy-5-(((((S)-5-oxopyrrolidin-2-yl)methyl)amino)methyl)pyridin-2-yl)-[1,1′-biphenyl]-3-yl)-2-(2-((((R)-5-oxopyrrolidin-2-yl)methyl)amino)ethyl)-2,3-dihydro-1H-pyrrolo[3,4-c]pyridin-1-one;
6-(2,2′-dichloro-3′-(6-methoxy-5-(((((R)-5-oxopyrrolidin-2-yl)methyl)amino)methyl)pyridin-2-yl)-[1,1′-biphenyl]-3-yl)-2-(2-((((R)-5-oxopyrrolidin-2-yl)methyl)amino)ethyl)-2,3-dihydro-1H-pyrrolo[3,4-c]pyridin-1-one;
(S)-5-((((6-(2,2′-dichloro-3′-(2-(2-hydroxyethyl)-8-methoxy-1,2,3,4-tetrahydroisoquinolin-6-yl)-[1,1′-biphenyl]-3-yl)-2-methoxypyridin-3-yl)methyl)amino)methyl)pyrrolidin-2-one;
(R)-5-((((6-(2,2′-dichloro-3′-(2-(2-hydroxyethyl)-8-methoxy-1,2,3,4-tetrahydroisoquinolin-6-yl)-[1,1′-biphenyl]-3-yl)-2-methoxypyridin-3-yl)methyl)amino)methyl)pyrrolidin-2-one;
(S)-5-((((6-(2,2′-dichloro-3′-((R)-5-((((S)-5-oxopyrrolidin-2-yl)methyl)amino)-5,6,7,8-tetrahydroquinolin-2-yl)-[1,1′-biphenyl]-3-yl)-2-methoxypyridin-3-yl)methyl)amino)methyl)pyrrolidin-2-one;
(S)-5-((((6-(2,2′-dichloro-3′-((S)-5-((((R)-5-oxopyrrolidin-2-yl)methyl)amino)-5,6,7,8-tetrahydroquinolin-2-yl)-[1,1′-biphenyl]-3-yl)-2-methoxypyridin-3-yl)methyl)amino)methyl)pyrrolidin-2-one;
(S)-5-((((6-(2,2′-dichloro-3′-((R)-5-((((R)-5-oxopyrrolidin-2-yl)methyl)amino)-5,6,7,8-tetrahydroquinolin-2-yl)-[1,1′-biphenyl]-3-yl)-2-methoxypyridin-3-yl)methyl)amino)methyl)pyrrolidin-2-one;
(S)-5-((((6-(2,2′-dichloro-3′-((S)-5-((((S)-5-oxopyrrolidin-2-yl)methyl)amino)-5,6,7,8-tetrahydroquinolin-2-yl)-[1,1′-biphenyl]-3-yl)-2-methoxypyridin-3-yl)methyl)amino)methyl)pyrrolidin-2-one;
(R)-5-((((6-(2,2′-dichloro-3′-((R)-5-((((S)-5-oxopyrrolidin-2-yl)methyl)amino)-5,6,7,8-tetrahydroquinolin-2-yl)-[1,1′-biphenyl]-3-yl)-2-methoxypyridin-3-yl)methyl)amino)methyl)pyrrolidin-2-one;
(R)-5-((((6-(2,2′-dichloro-3′-((S)-5-((((R)-5-oxopyrrolidin-2-yl)methyl)amino)-5,6,7,8-tetrahydroquinolin-2-yl)-[1,1′-biphenyl]-3-yl)-2-methoxypyridin-3-yl)methyl)amino)methyl)pyrrolidin-2-one;
(R)-5-((((6-(2,2′-dichloro-3′-((R)-5-((((R)-5-oxopyrrolidin-2-yl)methyl)amino)-5,6,7,8-tetrahydroquinolin-2-yl)-[1,1′-biphenyl]-3-yl)-2-methoxypyridin-3-yl)methyl)amino)methyl)pyrrolidin-2-one;
(R)-5-((((6-(2,2′-dichloro-3′-((S)-5-((((S)-5-oxopyrrolidin-2-yl)methyl)amino)-5,6,7,8-tetrahydroquinolin-2-yl)-[1,1′-biphenyl]-3-yl)-2-methoxypyridin-3-yl)methyl)amino)methyl)pyrrolidin-2-one;
(S)-2-(((1-acetylpiperidin-4-yl)amino)methyl)-6-(2,2′-dichloro-3′-(6-methoxy-5-((((5-oxopyrrolidin-2-yl)methyl)amino)methyl)pyridin-2-yl)-[1,1′-biphenyl]-3-yl)-3-methylpyrrolo[2,1-f][1,2,4]triazin-4(3H)-one;
(R)-2-(((1-acetylpiperidin-4-yl)amino)methyl)-6-(2,2′-dichloro-3′-(6-methoxy-5-((((5-oxopyrrolidin-2-yl)methyl)amino)methyl)pyridin-2-yl)-[1,1′-biphenyl]-3-yl)-3-methylpyrrolo[2,1-f][1,2,4]triazin-4(3H)-one;
(S)-6-(2,2′-dichloro-3′-(6-methoxy-5-((((5-oxopyrrolidin-2-yl)methyl)amino)methyl)pyridin-2-yl)-[1,1′-biphenyl]-3-yl)-3-methyl-2-(((2-(2-oxopyrrolidin-1-yl)ethyl)amino)methyl)pyrrolo[2,1-f][1,2,4]triazin-4(3H)-one;
(R)-6-(2,2′-dichloro-3′-(6-methoxy-5-((((5-oxopyrrolidin-2-yl)methyl)amino)methyl)pyridin-2-yl)-[1,1′-biphenyl]-3-yl)-3-methyl-2-(((2-(2-oxopyrrolidin-1-yl)ethyl)amino)methyl)pyrrolo[2,1-f][1,2,4]triazin-4(3H)-one;
(S)-5-((((6-(2,2′-dichloro-3′-(2-(((((S)-5-oxopyrrolidin-2-yl)methyl)amino)methyl)furo[2,3-b]pyridin-5-yl)-[1,1′-biphenyl]-3-yl)-2-methoxypyridin-3-yl)methyl)amino)methyl)pyrrolidin-2-one;
(R)-5-((((6-(2,2′-dichloro-3′-(2-(((((S)-5-oxopyrrolidin-2-yl)methyl)amino)methyl)furo[2,3-b]pyridin-5-yl)-[1,1′-biphenyl]-3-yl)-2-methoxypyridin-3-yl)methyl)amino)methyl)pyrrolidin-2-one;
(S)-5-((((6-(2,2′-dichloro-3′-(2-(((((R)-5-oxopyrrolidin-2-yl)methyl)amino)methyl)furo[2,3-b]pyridin-5-yl)-[1,1′-biphenyl]-3-yl)-2-methoxypyridin-3-yl)methyl)amino)methyl)pyrrolidin-2-one;
(R)-5-((((6-(2,2′-dichloro-3′-(2-(((((R)-5-oxopyrrolidin-2-yl)methyl)amino)methyl)furo[2,3-b]pyridin-5-yl)-[1,1′-biphenyl]-3-yl)-2-methoxypyridin-3-yl)methyl)amino)methyl)pyrrolidin-2-one;
(S)-2-((6-acetyl-2,6-diazaspiro[3.3]heptan-2-yl)methyl)-6-(2,2′-dichloro-3′-(6-methoxy-5-((((5-oxopyrrolidin-2-yl)methyl)amino)methyl)pyridin-2-yl)-[1,1′-biphenyl]-3-yl)-3-methylpyrrolo[2,1-f][1,2,4]triazin-4(3H)-one;
(R)-2-((6-acetyl-2,6-diazaspiro[3.3]heptan-2-yl)methyl)-6-(2,2′-dichloro-3′-(6-methoxy-5-((((5-oxopyrrolidin-2-yl)methyl)amino)methyl)pyridin-2-yl)-[1,1′-biphenyl]-3-yl)-3-methylpyrrolo[2,1-f][1,2,4]triazin-4(3H)-one;
(S)-6-(2,2′-dichloro-3′-(6-methoxy-5-((((5-oxopyrrolidin-2-yl)methyl)amino)methyl)pyridin-2-yl)-[1,1′-biphenyl]-3-yl)-2-((3-(2-hydroxypropan-2-yl)azetidin-1-yl)methyl)-3-methylpyrrolo[2,1-f][1,2,4]triazin-4(3H)-one;
(R)-6-(2,2′-dichloro-3′-(6-methoxy-5-((((5-oxopyrrolidin-2-yl)methyl)amino)methyl)pyridin-2-yl)-[1,1′-biphenyl]-3-yl)-2-((3-(2-hydroxypropan-2-yl)azetidin-1-yl)methyl)-3-methylpyrrolo[2,1-f][1,2,4]triazin-4(3H)-one;
(S)-6-(2,2′-dichloro-3′-(6-methoxy-5-((((5-oxopyrrolidin-2-yl)methyl)amino)methyl)pyridin-2-yl)-[1,1′-biphenyl]-3-yl)-2-(((2-fluoro-2-methylpropyl)amino)methyl)-3-methylpyrrolo[2,1-f][1,2,4]triazin-4(3H)-one;
(R)-6-(2,2′-dichloro-3′-(6-methoxy-5-((((5-oxopyrrolidin-2-yl)methyl)amino)methyl)pyridin-2-yl)-[1,1′-biphenyl]-3-yl)-2-(((2-fluoro-2-methylpropyl)amino)methyl)-3-methylpyrrolo[2,1-f][1,2,4]triazin-4(3H)-one;
(S)-6-(2,2′-dichloro-3′-(6-methoxy-5-((((5-oxopyrrolidin-2-yl)methyl)amino)methyl)pyridin-2-yl)-[1,1′-biphenyl]-3-yl)-2-((((1-fluorocyclopropyl)methyl)amino)methyl)-3-methylpyrrolo[2,1-f][1,2,4]triazin-4(3H)-one;
(R)-6-(2,2′-dichloro-3′-(6-methoxy-5-((((5-oxopyrrolidin-2-yl)methyl)amino)methyl)pyridin-2-yl)-[1,1′-biphenyl]-3-yl)-2-((((1-fluorocyclopropyl)methyl)amino)methyl)-3-methylpyrrolo[2,1-f][1,2,4]triazin-4(3H)-one;
6-(2,2′-dichloro-3′-(4-chloro-6-methoxy-5-(((((S)-5-oxopyrrolidin-2-yl)methyl)amino)methyl)pyridin-2-yl)-[1,1′-biphenyl]-3-yl)-2-((((1r,3r)-3-hydroxy-3-methylcyclobutyl)amino)methyl)-3-methylpyrrolo[2,1-f][1,2,4]triazin-4(3H)-one;
6-(2,2′-dichloro-3′-(4-chloro-6-methoxy-5-(((((S)-5-oxopyrrolidin-2-yl)methyl)amino)methyl)pyridin-2-yl)-[1,1′-biphenyl]-3-yl)-2-((((1s,3s)-3-hydroxy-3-methylcyclobutyl)amino)methyl)-3-methylpyrrolo[2,1-f][1,2,4]triazin-4(3H)-one;
6-(2,2′-dichloro-3′-(4-chloro-6-methoxy-5-(((((R)-5-oxopyrrolidin-2-yl)methyl)amino)methyl)pyridin-2-yl)-[1,1′-biphenyl]-3-yl)-2-((((1r,3r)-3-hydroxy-3-methylcyclobutyl)amino)methyl)-3-methylpyrrolo[2,1-f][1,2,4]triazin-4(3H)-one;
6-(2,2′-dichloro-3′-(4-chloro-6-methoxy-5-(((((R)-5-oxopyrrolidin-2-yl)methyl)amino)methyl)pyridin-2-yl)-[1,1′-biphenyl]-3-yl)-2-((((1s,3s)-3-hydroxy-3-methylcyclobutyl)amino)methyl)-3-methylpyrrolo[2,1-f][1,2,4]triazin-4(3H)-one;
(S)-6-(2,2′-dichloro-3′-(6-methoxy-5-((((5-oxopyrrolidin-2-yl)methyl)amino)methyl)pyridin-2-yl)-[1,1′-biphenyl]-3-yl)-2-(((3,3-difluoropropyl)amino)methyl)-3-methylpyrrolo[2,1-f][1,2,4]triazin-4(3H)-one;
(R)-6-(2,2′-dichloro-3′-(6-methoxy-5-((((5-oxopyrrolidin-2-yl)methyl)amino)methyl)pyridin-2-yl)-[1,1′-biphenyl]-3-yl)-2-(((3,3-difluoropropyl)amino)methyl)-3-methylpyrrolo[2,1-f][1,2,4]triazin-4(3H)-one;
(S)-6-(2,2′-dichloro-3′-(6-methoxy-5-((((5-oxopyrrolidin-2-yl)methyl)amino)methyl)pyridin-2-yl)-[1,1′-biphenyl]-3-yl)-3-methyl-2-(((3,3,3-trifluoro-2,2-dimethylpropyl)amino)methyl)pyrrolo[2,1-f][1,2,4]triazin-4(3H)-one;
(R)-6-(2,2′-dichloro-3′-(6-methoxy-5-((((5-oxopyrrolidin-2-yl)methyl)amino)methyl)pyridin-2-yl)-[1,1′-biphenyl]-3-yl)-3-methyl-2-(((3,3,3-trifluoro-2,2-dimethylpropyl)amino)methyl)pyrrolo[2,1-f][1,2,4]triazin-4(3H)-one;
6-(2,2′-dichloro-3′-(6-methoxy-5-(((((S)-5-oxopyrrolidin-2-yl)methyl)amino)methyl)pyridin-2-yl)-[1,1′-biphenyl]-3-yl)-2-(2-((((S)-5-oxopyrrolidin-2-yl)methyl)amino)ethyl)-1,2-dihydro-3H-pyrrolo[3,4-c]pyridin-3-one;
6-(2,2′-dichloro-3′-(6-methoxy-5-(((((R)-5-oxopyrrolidin-2-yl)methyl)amino)methyl)pyridin-2-yl)-[1,1′-biphenyl]-3-yl)-2-(2-((((S)-5-oxopyrrolidin-2-yl)methyl)amino)ethyl)-1,2-dihydro-3H-pyrrolo[3,4-c]pyridin-3-one;
6-(2,2′-dichloro-3′-(6-methoxy-5-(((((S)-5-oxopyrrolidin-2-yl)methyl)amino)methyl)pyridin-2-yl)-[1,1′-biphenyl]-3-yl)-2-(2-((((R)-5-oxopyrrolidin-2-yl)methyl)amino)ethyl)-1,2-dihydro-3H-pyrrolo[3,4-c]pyridin-3-one;
6-(2,2′-dichloro-3′-(6-methoxy-5-(((((R)-5-oxopyrrolidin-2-yl)methyl)amino)methyl)pyridin-2-yl)-[1,1′-biphenyl]-3-yl)-2-(2-((((R)-5-oxopyrrolidin-2-yl)methyl)amino)ethyl)-1,2-dihydro-3H-pyrrolo[3,4-c]pyridin-3-one;
(S)-6-(2,2′-dichloro-3′-(6-methoxy-5-((((5-oxopyrrolidin-2-yl)methyl)amino)methyl)pyridin-2-yl)-[1,1′-biphenyl]-3-yl)-2-((6-hydroxy-2-azaspiro[3.3]heptan-2-yl)methyl)-3-methylpyrrolo[2,1-f][1,2,4]triazin-4(3H)-one;
(R)-6-(2,2′-dichloro-3′-(6-methoxy-5-((((5-oxopyrrolidin-2-yl)methyl)amino)methyl)pyridin-2-yl)-[1,1′-biphenyl]-3-yl)-2-((6-hydroxy-2-azaspiro[3.3]heptan-2-yl)methyl)-3-methylpyrrolo[2,1-f][1,2,4]triazin-4(3H)-one;
(S)-6-(2,2′-dichloro-3′-(6-methoxy-5-((((5-oxopyrrolidin-2-yl)methyl)amino)methyl)pyridin-2-yl)-[1,1′-biphenyl]-3-yl)-2-((3-hydroxy-3-methylazetidin-1-yl)methyl)-3-methylpyrrolo[2,1-f][1,2,4]triazin-4(3H)-one;
(R)-6-(2,2′-dichloro-3′-(6-methoxy-5-((((5-oxopyrrolidin-2-yl)methyl)amino)methyl)pyridin-2-yl)-[1,1′-biphenyl]-3-yl)-2-((3-hydroxy-3-methylazetidin-1-yl)methyl)-3-methylpyrrolo[2,1-f][1,2,4]triazin-4(3H)-one;
(S)-1-((6-(2,2′-dichloro-3′-(6-methoxy-5-((((5-oxopyrrolidin-2-yl)methyl)amino)methyl)pyridin-2-yl)-[1,1′-biphenyl]-3-yl)-3-methyl-4-oxo-3,4-dihydropyrrolo[2,1-f][1,2,4]triazin-2-yl)methyl)-3-methylazetidine-3-carbonitrile;
(R)-1-((6-(2,2′-dichloro-3′-(6-methoxy-5-((((5-oxopyrrolidin-2-yl)methyl)amino)methyl)pyridin-2-yl)-[1,1′-biphenyl]-3-yl)-3-methyl-4-oxo-3,4-dihydropyrrolo[2,1-f][1,2,4]triazin-2-yl)methyl)-3-methylazetidine-3-carbonitrile;
(S)-5-((((6-(2,2′-dichloro-3′-(4-methoxy-6-(((S)-5-oxopyrrolidin-2-yl)methyl)-5,6,7,8-tetrahydropyrido[4,3-d]pyrimidin-2-yl)-[1,1′-biphenyl]-3-yl)-2-methoxypyridin-3-yl)methyl)amino)methyl)pyrrolidin-2-one;
(R)-5-((((6-(2,2′-dichloro-3′-(4-methoxy-6-(((S)-5-oxopyrrolidin-2-yl)methyl)-5,6,7,8-tetrahydropyrido[4,3-d]pyrimidin-2-yl)-[1,1′-biphenyl]-3-yl)-2-methoxypyridin-3-yl)methyl)amino)methyl)pyrrolidin-2-one;
(S)-5-((((6-(2,2′-dichloro-3′-(4-methoxy-6-(((R)-5-oxopyrrolidin-2-yl)methyl)-5,6,7,8-tetrahydropyrido[4,3-d]pyrimidin-2-yl)-[1,1′-biphenyl]-3-yl)-2-methoxypyridin-3-yl)methyl)amino)methyl)pyrrolidin-2-one;
(R)-5-((((6-(2,2′-dichloro-3′-(4-methoxy-6-(((R)-5-oxopyrrolidin-2-yl)methyl)-5,6,7,8-tetrahydropyrido[4,3-d]pyrimidin-2-yl)-[1,1′-biphenyl]-3-yl)-2-methoxypyridin-3-yl)methyl)amino)methyl)pyrrolidin-2-one;
6-(2,2′-dichloro-3′-(6-methoxy-5-(((((S)-5-oxopyrrolidin-2-yl)methyl)amino)methyl)pyridin-2-yl)-[1,1′-biphenyl]-3-yl)-3-methyl-2-(((((S)-oxetan-2-yl)methyl)amino)methyl)pyrrolo[2,1-f][1,2,4]triazin-4(3H)-one;
6-(2,2′-dichloro-3′-(6-methoxy-5-(((((R)-5-oxopyrrolidin-2-yl)methyl)amino)methyl)pyridin-2-yl)-[1,1′-biphenyl]-3-yl)-3-methyl-2-(((((S)-oxetan-2-yl)methyl)amino)methyl)pyrrolo[2,1-f][1,2,4]triazin-4(3H)-one;
6-(2,2′-dichloro-3′-(6-methoxy-5-(((((S)-5-oxopyrrolidin-2-yl)methyl)amino)methyl)pyridin-2-yl)-[1,1′-biphenyl]-3-yl)-3-methyl-2-(((((R)-oxetan-2-yl)methyl)amino)methyl)pyrrolo[2,1-f][1,2,4]triazin-4(3H)-one;
6-(2,2′-dichloro-3′-(6-methoxy-5-(((((R)-5-oxopyrrolidin-2-yl)methyl)amino)methyl)pyridin-2-yl)-[1,1′-biphenyl]-3-yl)-3-methyl-2-(((((R)-oxetan-2-yl)methyl)amino)methyl)pyrrolo[2,1-f][1,2,4]triazin-4(3H)-one;
(S)-6-(2,2′-dichloro-3′-(6-methoxy-5-((((5-oxopyrrolidin-2-yl)methyl)amino)methyl)pyridin-2-yl)-[1,1′-biphenyl]-3-yl)-2-(((3-hydroxybicyclo[1.1.1]pentan-1-yl)amino)methyl)-3-methylpyrrolo[2,1-f][1,2,4]triazin-4(3H)-one;
(R)-6-(2,2′-dichloro-3′-(6-methoxy-5-((((5-oxopyrrolidin-2-yl)methyl)amino)methyl)pyridin-2-yl)-[1,1′-biphenyl]-3-yl)-2-(((3-hydroxybicyclo[1.1.1]pentan-1-yl)amino)methyl)-3-methylpyrrolo[2,1-f][1,2,4]triazin-4(3H)-one;
6-(2,2′-dichloro-3′-(6-methoxy-5-(((((S)-5-oxopyrrolidin-2-yl)methyl)amino)methyl)pyridin-2-yl)-[1,1′-biphenyl]-3-yl)-3-methyl-2-(((((S)-tetrahydrofuran-2-yl)methyl)amino)methyl)pyrrolo[2,1-f][1,2,4]triazin-4(3H)-one;
6-(2,2′-dichloro-3′-(6-methoxy-5-(((((R)-5-oxopyrrolidin-2-yl)methyl)amino)methyl)pyridin-2-yl)-[1,1′-biphenyl]-3-yl)-3-methyl-2-(((((S)-tetrahydrofuran-2-yl)methyl)amino)methyl)pyrrolo[2,1-f][1,2,4]triazin-4(3H)-one;
6-(2,2′-dichloro-3′-(6-methoxy-5-(((((S)-5-oxopyrrolidin-2-yl)methyl)amino)methyl)pyridin-2-yl)-[1,1′-biphenyl]-3-yl)-3-methyl-2-(((((R)-tetrahydrofuran-2-yl)methyl)amino)methyl)pyrrolo[2,1-f][1,2,4]triazin-4(3H)-one;
6-(2,2′-dichloro-3′-(6-methoxy-5-(((((R)-5-oxopyrrolidin-2-yl)methyl)amino)methyl)pyridin-2-yl)-[1,1′-biphenyl]-3-yl)-3-methyl-2-(((((R)-tetrahydrofuran-2-yl)methyl)amino)methyl)pyrrolo[2,1-f][1,2,4]triazin-4(3H)-one;
6-(2,2′-dichloro-3′-(6-methoxy-5-(((((S)-5-oxopyrrolidin-2-yl)methyl)amino)methyl)pyridin-2-yl)-[1,1′-biphenyl]-3-yl)-2-((((S)-2-methoxypropyl)amino)methyl)-3-methylpyrrolo[2,1-f][1,2,4]triazin-4(3H)-one;
6-(2,2′-dichloro-3′-(6-methoxy-5-(((((R)-5-oxopyrrolidin-2-yl)methyl)amino)methyl)pyridin-2-yl)-[1,1′-biphenyl]-3-yl)-2-((((S)-2-methoxypropyl)amino)methyl)-3-methylpyrrolo[2,1-f][1,2,4]triazin-4(3H)-one;
6-(2,2′-dichloro-3′-(6-methoxy-5-(((((S)-5-oxopyrrolidin-2-yl)methyl)amino)methyl)pyridin-2-yl)-[1,1′-biphenyl]-3-yl)-2-((((R)-2-methoxypropyl)amino)methyl)-3-methylpyrrolo[2,1-f][1,2,4]triazin-4(3H)-one;
6-(2,2′-dichloro-3′-(6-methoxy-5-(((((R)-5-oxopyrrolidin-2-yl)methyl)amino)methyl)pyridin-2-yl)-[1,1′-biphenyl]-3-yl)-2-((((R)-2-methoxypropyl)amino)methyl)-3-methylpyrrolo[2,1-f][1,2,4]triazin-4(3H)-one;
6-(2,2′-dichloro-3′-(6-methoxy-5-(((((S)-5-oxopyrrolidin-2-yl)methyl)amino)methyl)pyridin-2-yl)-[1,1′-biphenyl]-3-yl)-3-methyl-2-(((((S)-5-oxopyrrolidin-3-yl)methyl)amino)methyl)pyrrolo[2,1-f][1,2,4]triazin-4(3H)-one;
6-(2,2′-dichloro-3′-(6-methoxy-5-(((((R)-5-oxopyrrolidin-2-yl)methyl)amino)methyl)pyridin-2-yl)-[1,1′-biphenyl]-3-yl)-3-methyl-2-(((((S)-5-oxopyrrolidin-3-yl)methyl)amino)methyl)pyrrolo[2,1-f][1,2,4]triazin-4(3H)-one;
6-(2,2′-dichloro-3′-(6-methoxy-5-(((((S)-5-oxopyrrolidin-2-yl)methyl)amino)methyl)pyridin-2-yl)-[1,1′-biphenyl]-3-yl)-3-methyl-2-(((((R)-5-oxopyrrolidin-3-yl)methyl)amino)methyl)pyrrolo[2,1-f][1,2,4]triazin-4(3H)-one;
6-(2,2′-dichloro-3′-(6-methoxy-5-(((((R)-5-oxopyrrolidin-2-yl)methyl)amino)methyl)pyridin-2-yl)-[1,1′-biphenyl]-3-yl)-3-methyl-2-(((((R)-5-oxopyrrolidin-3-yl)methyl)amino)methyl)pyrrolo[2,1-f][1,2,4]triazin-4(3H)-one;
(S)-6-(2,2′-dichloro-3′-(6-methoxy-5-((((5-oxopyrrolidin-2-yl)methyl)amino)methyl)pyridin-2-yl)-[1,1′-biphenyl]-3-yl)-2-((isopropylamino)methyl)-3-methylpyrrolo[2,1-f][1,2,4]triazin-4(3H)-one;
(R)-6-(2,2′-dichloro-3′-(6-methoxy-5-((((5-oxopyrrolidin-2-yl)methyl)amino)methyl)pyridin-2-yl)-[1,1′-biphenyl]-3-yl)-2-((isopropylamino)methyl)-3-methylpyrrolo[2,1-f][1,2,4]triazin-4(3H)-one;
(S)-6-(2,2′-dichloro-3′-(4-chloro-6-methoxy-5-((((5-oxopyrrolidin-2-yl)methyl)amino)methyl)pyridin-2-yl)-[1,1′-biphenyl]-3-yl)-2-(((2-hydroxy-2-methylpropyl)amino)methyl)-3-methylpyrrolo[2,1-f][1,2,4]triazin-4(3H)-one;
(R)-6-(2,2′-dichloro-3′-(4-chloro-6-methoxy-5-((((5-oxopyrrolidin-2-yl)methyl)amino)methyl)pyridin-2-yl)-[1,1′-biphenyl]-3-yl)-2-(((2-hydroxy-2-methylpropyl)amino)methyl)-3-methylpyrrolo[2,1-f][1,2,4]triazin-4(3H)-one;
(S)-2-((tert-butylamino)methyl)-6-(2,2′-dichloro-3′-(6-methoxy-5-((((5-oxopyrrolidin-2-yl)methyl)amino)methyl)pyridin-2-yl)-[1,1′-biphenyl]-3-yl)-3-methylpyrrolo[2,1-f][1,2,4]triazin-4(3H)-one;
(R)-2-((tert-butylamino)methyl)-6-(2,2′-dichloro-3′-(6-methoxy-5-((((5-oxopyrrolidin-2-yl)methyl)amino)methyl)pyridin-2-yl)-[1,1′-biphenyl]-3-yl)-3-methylpyrrolo[2,1-f][1,2,4]triazin-4(3H)-one;
6-(2,2′-dichloro-3′-(4-fluoro-6-methoxy-5-(((((S)-5-oxopyrrolidin-2-yl)methyl)amino)methyl)pyridin-2-yl)-[1,1′-biphenyl]-3-yl)-2-((((1r,3r)-3-hydroxy-3-methylcyclobutyl)amino)methyl)-3-methylpyrrolo[2,1-f][1,2,4]triazin-4(3H)-one;
6-(2,2′-dichloro-3′-(4-fluoro-6-methoxy-5-(((((R)-5-oxopyrrolidin-2-yl)methyl)amino)methyl)pyridin-2-yl)-[1,1′-biphenyl]-3-yl)-2-((((1r,3r)-3-hydroxy-3-methylcyclobutyl)amino)methyl)-3-methylpyrrolo[2,1-f][1,2,4]triazin-4(3H)-one;
6-(2,2′-dichloro-3′-(4-fluoro-6-methoxy-5-(((((S)-5-oxopyrrolidin-2-yl)methyl)amino)methyl)pyridin-2-yl)-[1,1′-biphenyl]-3-yl)-2-((((1s,3s)-3-hydroxy-3-methylcyclobutyl)amino)methyl)-3-methylpyrrolo[2,1-f][1,2,4]triazin-4(3H)-one;
6-(2,2′-dichloro-3′-(4-fluoro-6-methoxy-5-(((((R)-5-oxopyrrolidin-2-yl)methyl)amino)methyl)pyridin-2-yl)-[1,1′-biphenyl]-3-yl)-2-((((1s,3s)-3-hydroxy-3-methylcyclobutyl)amino)methyl)-3-methylpyrrolo[2,1-f][1,2,4]triazin-4(3H)-one;
(S)-4-(((6-(2,2′-dichloro-3′-(6-methoxy-5-(((((S)-5-oxopyrrolidin-2-yl)methyl)amino)methyl)pyridin-2-yl)-[1,1′-biphenyl]-3-yl)-3-methyl-4-oxo-3,4-dihydropyrrolo[2,1-f][1,2,4]triazin-2-yl)methyl)amino)-3-hydroxy-N,N-dimethylbutanamide;
(R)-4-(((6-(2,2′-dichloro-3′-(6-methoxy-5-(((((S)-5-oxopyrrolidin-2-yl)methyl)amino)methyl)pyridin-2-yl)-[1,1′-biphenyl]-3-yl)-3-methyl-4-oxo-3,4-dihydropyrrolo[2,1-f][1,2,4]triazin-2-yl)methyl)amino)-3-hydroxy-N,N-dimethylbutanamide;
(S)-4-(((6-(2,2′-dichloro-3′-(6-methoxy-5-(((((R)-5-oxopyrrolidin-2-yl)methyl)amino)methyl)pyridin-2-yl)-[1,1′-biphenyl]-3-yl)-3-methyl-4-oxo-3,4-dihydropyrrolo[2,1-f][1,2,4]triazin-2-yl)methyl)amino)-3-hydroxy-N,N-dimethylbutanamide;
(R)-4-(((6-(2,2′-dichloro-3′-(6-methoxy-5-(((((R)-5-oxopyrrolidin-2-yl)methyl)amino)methyl)pyridin-2-yl)-[1,1′-biphenyl]-3-yl)-3-methyl-4-oxo-3,4-dihydropyrrolo[2,1-f][1,2,4]triazin-2-yl)methyl)amino)-3-hydroxy-N,N-dimethylbutanamide;
methyl (S)-((6-(2,2′-dichloro-3′-(6-methoxy-5-((((5-oxopyrrolidin-2-yl)methyl)amino)methyl)pyridin-2-yl)-[1,1′-biphenyl]-3-yl)-3-methyl-4-oxo-3,4-dihydropyrrolo[2,1-f][1,2,4]triazin-2-yl)methyl)glycinate;
methyl (R)-((6-(2,2′-dichloro-3′-(6-methoxy-5-((((5-oxopyrrolidin-2-yl)methyl)amino)methyl)pyridin-2-yl)-[1,1′-biphenyl]-3-yl)-3-methyl-4-oxo-3,4-dihydropyrrolo[2,1-f][1,2,4]triazin-2-yl)methyl)glycinate;
2-(2,2′-dichloro-3′-(6-methoxy-5-(((((S)-5-oxopyrrolidin-2-yl)methyl)amino)methyl)pyridin-2-yl)-[1,1′-biphenyl]-3-yl)-6-(((((S)-5-oxopyrrolidin-2-yl)methyl)amino)methyl)isoindolin-1-one;
2-(2,2′-dichloro-3′-(6-methoxy-5-(((((R)-5-oxopyrrolidin-2-yl)methyl)amino)methyl)pyridin-2-yl)-[1,1′-biphenyl]-3-yl)-6-(((((S)-5-oxopyrrolidin-2-yl)methyl)amino)methyl)isoindolin-1-one;
2-(2,2′-dichloro-3′-(6-methoxy-5-(((((S)-5-oxopyrrolidin-2-yl)methyl)amino)methyl)pyridin-2-yl)-[1,1′-biphenyl]-3-yl)-6-(((((R)-5-oxopyrrolidin-2-yl)methyl)amino)methyl)isoindolin-1-one;
2-(2,2′-dichloro-3′-(6-methoxy-5-(((((R)-5-oxopyrrolidin-2-yl)methyl)amino)methyl)pyridin-2-yl)-[1,1′-biphenyl]-3-yl)-6-(((((R)-5-oxopyrrolidin-2-yl)methyl)amino)methyl)isoindolin-1-one;
(S)-5-((((6-(2,2′-dichloro-3′-(2-(2-(1-hydroxycyclobutyl)ethyl)-8-methoxy-1,2,3,4-tetrahydroisoquinolin-6-yl)-[1,1′-biphenyl]-3-yl)-2-methoxypyridin-3-yl)methyl)amino)methyl)pyrrolidin-2-one;
(R)-5-((((6-(2,2′-dichloro-3′-(2-(2-(1-hydroxycyclobutyl)ethyl)-8-methoxy-1,2,3,4-tetrahydroisoquinolin-6-yl)-[1,1′-biphenyl]-3-yl)-2-methoxypyridin-3-yl)methyl)amino)methyl)pyrrolidin-2-one;
(S)-5-((((6-(2,2′-dichloro-3′-(2-(3-hydroxy-3-methylbutyl)-8-methoxy-1,2,3,4-tetrahydroisoquinolin-6-yl)-[1,1′-biphenyl]-3-yl)-2-methoxypyridin-3-yl)methyl)amino)methyl)pyrrolidin-2-one;
(R)-5-((((6-(2,2′-dichloro-3′-(2-(3-hydroxy-3-methylbutyl)-8-methoxy-1,2,3,4-tetrahydroisoquinolin-6-yl)-[1,1′-biphenyl]-3-yl)-2-methoxypyridin-3-yl)methyl)amino)methyl)pyrrolidin-2-one;
6-(2,2′-dichloro-3′-(6-methoxy-5-(((((S)-5-oxopyrrolidin-2-yl)methyl)amino)methyl)pyridin-2-yl)-[1,1′-biphenyl]-3-yl)-2-((((lr,4r)-4-hydroxycyclohexyl)amino)methyl)-3-methylpyrrolo[2,1-f][1,2,4]triazin-4(3H)-one;
6-(2,2′-dichloro-3′-(6-methoxy-5-(((((S)-5-oxopyrrolidin-2-yl)methyl)amino)methyl)pyridin-2-yl)-[1,1′-biphenyl]-3-yl)-2-((((1s,4s)-4-hydroxycyclohexyl)amino)methyl)-3-methylpyrrolo[2,1-f][1,2,4]triazin-4(3H)-one;
6-(2,2′-dichloro-3′-(6-methoxy-5-(((((R)-5-oxopyrrolidin-2-yl)methyl)amino)methyl)pyridin-2-yl)-[1,1′-biphenyl]-3-yl)-2-((((lr,4r)-4-hydroxycyclohexyl)amino)methyl)-3-methylpyrrolo[2,1-f][1,2,4]triazin-4(3H)-one;
6-(2,2′-dichloro-3′-(6-methoxy-5-(((((R)-5-oxopyrrolidin-2-yl)methyl)amino)methyl)pyridin-2-yl)-[1,1′-biphenyl]-3-yl)-2-((((1s,4s)-4-hydroxycyclohexyl)amino)methyl)-3-methylpyrrolo[2,1-f][1,2,4]triazin-4(3H)-one;
(S)-((6-(2,2′-dichloro-3′-(6-methoxy-5-((((5-oxopyrrolidin-2-yl)methyl)amino)methyl)pyridin-2-yl)-[1,1′-biphenyl]-3-yl)-3-methyl-4-oxo-3,4-dihydropyrrolo[2,1-f][1,2,4]triazin-2-yl)methyl)glycine;
(R)-((6-(2,2′-dichloro-3′-(6-methoxy-5-((((5-oxopyrrolidin-2-yl)methyl)amino)methyl)pyridin-2-yl)-[1,1′-biphenyl]-3-yl)-3-methyl-4-oxo-3,4-dihydropyrrolo[2,1-f][1,2,4]triazin-2-yl)methyl)glycine;
(5S)-5-((((6-(2,2′-dichloro-3′-(2-((2S)-hydroxy-3-methylbutyl)-8-methoxy-1,2,3,4-tetrahydroisoquinolin-6-yl)-[1,1′-biphenyl]-3-yl)-2-methoxypyridin-3-yl)methyl)amino)methyl)pyrrolidin-2-one;
(5R)-5-((((6-(2,2′-dichloro-3′-(2-((2S)-hydroxy-3-methylbutyl)-8-methoxy-1,2,3,4-tetrahydroisoquinolin-6-yl)-[1,1′-biphenyl]-3-yl)-2-methoxypyridin-3-yl)methyl)amino)methyl)pyrrolidin-2-one;
(5S)-5-((((6-(2,2′-dichloro-3′-(2-((2R)-hydroxy-3-methylbutyl)-8-methoxy-1,2,3,4-tetrahydroisoquinolin-6-yl)-[1,1′-biphenyl]-3-yl)-2-methoxypyridin-3-yl)methyl)amino)methyl)pyrrolidin-2-one;
(5R)-5-((((6-(2,2′-dichloro-3′-(2-((2R)-hydroxy-3-methylbutyl)-8-methoxy-1,2,3,4-tetrahydroisoquinolin-6-yl)-[1,1′-biphenyl]-3-yl)-2-methoxypyridin-3-yl)methyl)amino)methyl)pyrrolidin-2-one;
methyl (S)-4-(((6-(2,2′-dichloro-3′-(6-methoxy-5-(((((S)-5-oxopyrrolidin-2-yl)methyl)amino)methyl)pyridin-2-yl)-[1,1′-biphenyl]-3-yl)-3-methyl-4-oxo-3,4-dihydropyrrolo[2,1-f][1,2,4]triazin-2-yl)methyl)amino)-3-hydroxybutanoate;
methyl (R)-4-(((6-(2,2′-dichloro-3′-(6-methoxy-5-(((((S)-5-oxopyrrolidin-2-yl)methyl)amino)methyl)pyridin-2-yl)-[1,1′-biphenyl]-3-yl)-3-methyl-4-oxo-3,4-dihydropyrrolo[2,1-f][1,2,4]triazin-2-yl)methyl)amino)-3-hydroxybutanoate;
methyl (S)-4-(((6-(2,2′-dichloro-3′-(6-methoxy-5-(((((R)-5-oxopyrrolidin-2-yl)methyl)amino)methyl)pyridin-2-yl)-[1,1′-biphenyl]-3-yl)-3-methyl-4-oxo-3,4-dihydropyrrolo[2,1-f][1,2,4]triazin-2-yl)methyl)amino)-3-hydroxybutanoate;
methyl (R)-4-(((6-(2,2′-dichloro-3′-(6-methoxy-5-(((((R)-5-oxopyrrolidin-2-yl)methyl)amino)methyl)pyridin-2-yl)-[1,1′-biphenyl]-3-yl)-3-methyl-4-oxo-3,4-dihydropyrrolo[2,1-f][1,2,4]triazin-2-yl)methyl)amino)-3-hydroxybutanoate;
(S)-6-(2,2′-dichloro-3′-(4-chloro-6-methoxy-5-((((5-oxopyrrolidin-2-yl)methyl)amino)methyl)pyridin-2-yl)-[1,1′-biphenyl]-3-yl)-2-(((3-fluoropropyl)amino)methyl)-3-methylpyrrolo[2,1-f][1,2,4]triazin-4(3H)-one;
(R)-6-(2,2′-dichloro-3′-(4-chloro-6-methoxy-5-((((5-oxopyrrolidin-2-yl)methyl)amino)methyl)pyridin-2-yl)-[1,1′-biphenyl]-3-yl)-2-(((3-fluoropropyl)amino)methyl)-3-methylpyrrolo[2,1-f][1,2,4]triazin-4(3H)-one;
(S)-6-(2,2′-dichloro-3′-(4-chloro-6-methoxy-5-((((5-oxopyrrolidin-2-yl)methyl)amino)methyl)pyridin-2-yl)-[1,1′-biphenyl]-3-yl)-2-(((2-hydroxyethyl)amino)methyl)-3-methylpyrrolo[2,1-f][1,2,4]triazin-4(3H)-one;
(R)-6-(2,2′-dichloro-3′-(4-chloro-6-methoxy-5-((((5-oxopyrrolidin-2-yl)methyl)amino)methyl)pyridin-2-yl)-[1,1′-biphenyl]-3-yl)-2-(((2-hydroxyethyl)amino)methyl)-3-methylpyrrolo[2,1-f][1,2,4]triazin-4(3H)-one;
(S)-6-(2,2′-dichloro-3′-(4-chloro-6-methoxy-5-((((5-oxopyrrolidin-2-yl)methyl)amino)methyl)pyridin-2-yl)-[1,1′-biphenyl]-3-yl)-2-((3-hydroxy-3-methylazetidin-1-yl)methyl)-3-methylpyrrolo[2,1-f][1,2,4]triazin-4(3H)-one;
(R)-6-(2,2′-dichloro-3′-(4-chloro-6-methoxy-5-((((5-oxopyrrolidin-2-yl)methyl)amino)methyl)pyridin-2-yl)-[1,1′-biphenyl]-3-yl)-2-((3-hydroxy-3-methylazetidin-1-yl)methyl)-3-methylpyrrolo[2,1-f][1,2,4]triazin-4(3H)-one;
(S)-4-(((6-(2,2′-dichloro-3′-(6-methoxy-5-(((((S)-5-oxopyrrolidin-2-yl)methyl)amino)methyl)pyridin-2-yl)-[1,1′-biphenyl]-3-yl)-3-methyl-4-oxo-3,4-dihydropyrrolo[2,1-f][1,2,4]triazin-2-yl)methyl)amino)-3-hydroxybutanoic acid;
(R)-4-(((6-(2,2′-dichloro-3′-(6-methoxy-5-(((((S)-5-oxopyrrolidin-2-yl)methyl)amino)methyl)pyridin-2-yl)-[1,1′-biphenyl]-3-yl)-3-methyl-4-oxo-3,4-dihydropyrrolo[2,1-f][1,2,4]triazin-2-yl)methyl)amino)-3-hydroxybutanoic acid;
(S)-4-(((6-(2,2′-dichloro-3′-(6-methoxy-5-(((((R)-5-oxopyrrolidin-2-yl)methyl)amino)methyl)pyridin-2-yl)-[1,1′-biphenyl]-3-yl)-3-methyl-4-oxo-3,4-dihydropyrrolo[2,1-f][1,2,4]triazin-2-yl)methyl)amino)-3-hydroxybutanoic acid;
(R)-4-(((6-(2,2′-dichloro-3′-(6-methoxy-5-(((((R)-5-oxopyrrolidin-2-yl)methyl)amino)methyl)pyridin-2-yl)-[1,1′-biphenyl]-3-yl)-3-methyl-4-oxo-3,4-dihydropyrrolo[2,1-f][1,2,4]triazin-2-yl)methyl)amino)-3-hydroxybutanoic acid;
(S)-1-((6-(2,2′-dichloro-3′-(6-methoxy-5-(((((S)-5-oxopyrrolidin-2-yl)methyl)amino)methyl)pyridin-2-yl)-[1,1′-biphenyl]-3-yl)-2-methoxypyridin-3-yl)methyl)piperidine-2-carboxylic acid;
(R)-1-((6-(2,2′-dichloro-3′-(6-methoxy-5-(((((S)-5-oxopyrrolidin-2-yl)methyl)amino)methyl)pyridin-2-yl)-[1,1′-biphenyl]-3-yl)-2-methoxypyridin-3-yl)methyl)piperidine-2-carboxylic acid;
(S)-1-((6-(2,2′-dichloro-3′-(6-methoxy-5-(((((R)-5-oxopyrrolidin-2-yl)methyl)amino)methyl)pyridin-2-yl)-[1,1′-biphenyl]-3-yl)-2-methoxypyridin-3-yl)methyl)piperidine-2-carboxylic acid;
(R)-1-((6-(2,2′-dichloro-3′-(6-methoxy-5-(((((R)-5-oxopyrrolidin-2-yl)methyl)amino)methyl)pyridin-2-yl)-[1,1′-biphenyl]-3-yl)-2-methoxypyridin-3-20 yl)methyl)piperidine-2-carboxylic acid;
ethyl (S)-3-(((6-(2,2′-dichloro-3′-(6-methoxy-5-((((5-oxopyrrolidin-2-yl)methyl)amino)methyl)pyridin-2-yl)-[1,1′-biphenyl]-3-yl)-3-methyl-4-oxo-3,4-dihydropyrrolo[2,1-f][1,2,4]triazin-2-yl)methyl)amino)-2,2-dimethylpropanoate;
methyl (R)-3-(((6-(2,2′-dichloro-3′-(6-methoxy-5-((((5-oxopyrrolidin-2-yl)methyl)amino)methyl)pyridin-2-yl)-[1,1′-biphenyl]-3-yl)-3-methyl-4-oxo-3,4-dihydropyrrolo[2,1-f][1,2,4]triazin-2-yl)methyl)amino)-2,2-dimethylpropanoate;
methyl (S)-1-((6-(2,2′-dichloro-3′-(6-methoxy-5-((((5-oxopyrrolidin-2-yl)methyl)amino)methyl)pyridin-2-yl)-[1,1′-biphenyl]-3-yl)-3-methyl-4-oxo-3,4-dihydropyrrolo[2,1-f][1,2,4]triazin-2-yl)methyl)azetidine-3-carboxylate;
methyl (R)-1-((6-(2,2′-dichloro-3′-(6-methoxy-5-((((5-oxopyrrolidin-2-yl)methyl)amino)methyl)pyridin-2-yl)-[1,1′-biphenyl]-3-yl)-3-methyl-4-oxo-3,4-dihydropyrrolo[2,1-f][1,2,4]triazin-2-yl)methyl)azetidine-3-carboxylate;
(S)-5-((((6-(2,2′-dichloro-3′-(8-methoxy-2-(2-(piperidin-4-yl)ethyl)-1,2,3,4-tetrahydroisoquinolin-6-yl)-[1,1′-biphenyl]-3-yl)-2-methoxypyridin-3-yl)methyl)amino)methyl)pyrrolidin-2-one;
(R)-5-((((6-(2,2′-dichloro-3′-(8-methoxy-2-(2-(piperidin-4-yl)ethyl)-1,2,3,4-tetrahydroisoquinolin-6-yl)-[1,1′-biphenyl]-3-yl)-2-methoxypyridin-3-yl)methyl)amino)methyl)pyrrolidin-2-one;
(S)-5-((((6-(3′-(2-((1-acetylpiperidin-4-yl)methyl)-8-methoxy-1,2,3,4-tetrahydroisoquinolin-6-yl)-2,2′-dichloro-[1,1′-biphenyl]-3-yl)-2-methoxypyridin-3-yl)methyl)amino)methyl)pyrrolidin-2-one;
(R)-5-((((6-(3′-(2-((1-acetylpiperidin-4-yl)methyl)-8-methoxy-1,2,3,4-tetrahydroisoquinolin-6-yl)-2,2′-dichloro-[1,1′-biphenyl]-3-yl)-2-methoxypyridin-3-yl)methyl)amino)methyl)pyrrolidin-2-one;
(S)-6-(2,2′-dichloro-3′-(4-fluoro-6-methoxy-5-((((5-oxopyrrolidin-2-yl)methyl)amino)methyl)pyridin-2-yl)-[1,1′-biphenyl]-3-yl)-2-((3-hydroxy-3-methylazetidin-1-yl)methyl)-3-methylpyrrolo[2,1-f][1,2,4]triazin-4(3H)-one;
(R)-6-(2,2′-dichloro-3′-(4-fluoro-6-methoxy-5-((((5-oxopyrrolidin-2-yl)methyl)amino)methyl)pyridin-2-yl)-[1,1′-biphenyl]-3-yl)-2-((3-hydroxy-3-methylazetidin-1-yl)methyl)-3-methylpyrrolo[2,1-f][1,2,4]triazin-4(3H)-one;
(S)-6-(2,2′-dichloro-3′-(4-fluoro-6-methoxy-5-((((5-oxopyrrolidin-2-yl)methyl)amino)methyl)pyridin-2-yl)-[1,1′-biphenyl]-3-yl)-2-(((2-hydroxyethyl)amino)methyl)-3-methylpyrrolo[2,1-f][1,2,4]triazin-4(3H)-one;
(R)-6-(2,2′-dichloro-3′-(4-fluoro-6-methoxy-5-((((5-oxopyrrolidin-2-yl)methyl)amino)methyl)pyridin-2-yl)-[1,1′-biphenyl]-3-yl)-2-(((2-hydroxyethyl)amino)methyl)-3-methylpyrrolo[2,1-f][1,2,4]triazin-4(3H)-one;
(S)-1-((6-(2,2′-dichloro-3′-(6-methoxy-5-(((((S)-5-oxopyrrolidin-2-yl)methyl)amino)methyl)pyridin-2-yl)-[1,1′-biphenyl]-3-yl)-3-methyl-4-oxo-3,4-dihydropyrrolo[2,1-f][1,2,4]triazin-2-yl)methyl)pyrrolidine-3-carboxylic acid;
(R)-1-((6-(2,2′-dichloro-3′-(6-methoxy-5-(((((S)-5-oxopyrrolidin-2-yl)methyl)amino)methyl)pyridin-2-yl)-[1,1′-biphenyl]-3-yl)-3-methyl-4-oxo-3,4-dihydropyrrolo[2,1-f][1,2,4]triazin-2-yl)methyl)pyrrolidine-3-carboxylic acid;
(S)-1-((6-(2,2′-dichloro-3′-(6-methoxy-5-(((((R)-5-oxopyrrolidin-2-yl)methyl)amino)methyl)pyridin-2-yl)-[1,1′-biphenyl]-3-yl)-3-methyl-4-oxo-3,4-dihydropyrrolo[2,1-f][1,2,4]triazin-2-yl)methyl)pyrrolidine-3-carboxylic acid;
(R)-1-((6-(2,2′-dichloro-3′-(6-methoxy-5-(((((R)-5-oxopyrrolidin-2-yl)methyl)amino)methyl)pyridin-2-yl)-[1,1′-biphenyl]-3-yl)-3-methyl-4-oxo-3,4-dihydropyrrolo[2,1-f][1,2,4]triazin-2-yl)methyl)pyrrolidine-3-carboxylic acid;
(S)-1-(((6-(2,2′-dichloro-3′-(6-methoxy-5-((((5-oxopyrrolidin-2-yl)methyl)amino)methyl)pyridin-2-yl)-[1,1′-biphenyl]-3-yl)-3-methyl-4-oxo-3,4-dihydropyrrolo[2,1-f][1,2,4]triazin-2-yl)methyl)amino)cyclopropane-1-carboxylic acid;
(R)-1-(((6-(2,2′-dichloro-3′-(6-methoxy-5-((((5-oxopyrrolidin-2-yl)methyl)amino)methyl)pyridin-2-yl)-[1,1′-biphenyl]-3-yl)-3-methyl-4-oxo-3,4-dihydropyrrolo[2,1-f][1,2,4]triazin-2-yl)methyl)amino)cyclopropane-1-carboxylic acid;
(S)-3-(((6-(2,2′-dichloro-3′-(6-methoxy-5-((((5-oxopyrrolidin-2-yl)methyl)amino)methyl)pyridin-2-yl)-[1,1′-biphenyl]-3-yl)-3-methyl-4-oxo-3,4-dihydropyrrolo[2,1-f][1,2,4]triazin-2-yl)methyl)amino)-2,2-dimethylpropanoic acid;
(R)-3-(((6-(2,2′-dichloro-3′-(6-methoxy-5-((((5-oxopyrrolidin-2-yl)methyl)amino)methyl)pyridin-2-yl)-[1,1′-biphenyl]-3-yl)-3-methyl-4-oxo-3,4-dihydropyrrolo[2,1-f][1,2,4]triazin-2-yl)methyl)amino)-2,2-dimethylpropanoic acid;
methyl ((6-(2,2′-dichloro-3′-(6-methoxy-5-(((((S)-5-oxopyrrolidin-2-yl)methyl)amino)methyl)pyridin-2-yl)-[1,1′-biphenyl]-3-yl)-3-methyl-4-oxo-3,4-dihydropyrrolo[2,1-f][1,2,4]triazin-2-yl)methyl)-D-alaninate;
methyl ((6-(2,2′-dichloro-3′-(6-methoxy-5-(((((R)-5-oxopyrrolidin-2-yl)methyl)amino)methyl)pyridin-2-yl)-[1,1′-biphenyl]-3-yl)-3-methyl-4-oxo-3,4-dihydropyrrolo[2,1-f][1,2,4]triazin-2-yl)methyl)-D-alaninate;
methyl ((6-(2,2′-dichloro-3′-(6-methoxy-5-(((((S)-5-oxopyrrolidin-2-yl)methyl)amino)methyl)pyridin-2-yl)-[1,1′-biphenyl]-3-yl)-3-methyl-4-oxo-3,4-dihydropyrrolo[2,1-f][1,2,4]triazin-2-yl)methyl)-L-alaninate;
methyl ((6-(2,2′-dichloro-3′-(6-methoxy-5-(((((R)-5-oxopyrrolidin-2-yl)methyl)amino)methyl)pyridin-2-yl)-[1,1′-biphenyl]-3-yl)-3-methyl-4-oxo-3,4-dihydropyrrolo[2,1-f][1,2,4]triazin-2-yl)methyl)-L-alaninate;
(S)-1-((6-(2,2′-dichloro-3′-(6-methoxy-5-((((5-oxopyrrolidin-2-yl)methyl)amino)methyl)pyridin-2-yl)-[1,1′-biphenyl]-3-yl)-3-methyl-4-oxo-3,4-dihydropyrrolo[2,1-f][1,2,4]triazin-2-yl)methyl)azetidine-3-carboxylic acid;
(R)-1-((6-(2,2′-dichloro-3′-(6-methoxy-5-((((5-oxopyrrolidin-2-yl)methyl)amino)methyl)pyridin-2-yl)-[1,1′-biphenyl]-3-yl)-3-methyl-4-oxo-3,4-dihydropyrrolo[2,1-f][1,2,4]triazin-2-yl)methyl)azetidine-3-carboxylic acid;
(5S)-5-[[[6-[2-chloro-3-[2-chloro-3-[2-[[[(2S)-5-oxopyrrolidin-2-yl]methylamino]methyl]-[1,2,4]triazolo[1,5-a]pyrimidin-6-yl]phenyl]phenyl]-2-methoxy-3-pyridyl]methylamino] methyl]pyrrolidin-2-one;
(5R)-5-[[[6-[2-chloro-3-[2-chloro-3-[2-[[[(2S)-5-oxopyrrolidin-2-yl]methylamino]methyl]-[1,2,4]triazolo[1,5-a]pyrimidin-6-yl]phenyl]phenyl]-2-methoxy-3-pyridyl]methylamino] methyl]pyrrolidin-2-one;
(5S)-5-[[[6-[2-chloro-3-[2-chloro-3-[2-[[[(2R)-5-oxopyrrolidin-2-yl]methylamino]methyl]-[1,2,4]triazolo[1,5-a]pyrimidin-6-yl]phenyl]phenyl]-2-methoxy-3-pyridyl]methylamino] methyl]pyrrolidin-2-one;
(5R)-5-[[[6-[2-chloro-3-[2-chloro-3-[2-[[[(2R)-5-oxopyrrolidin-2-yl]methylamino]methyl]-[1,2,4]triazolo[1,5-a]pyrimidin-6-yl]phenyl]phenyl]-2-methoxy-3-pyridyl]methylamino] methyl]pyrrolidin-2-one;
(2R)-2-[[6-[2-chloro-3-[2-chloro-3-[6-methoxy-5-[[[(2S)-5-oxopyrrolidin-2-yl]methylamino] methyl]-2-pyridyl]phenyl]phenyl]-3-methyl-4-oxo-pyrrolo[2,1-f][1,2,4]triazin-2-yl]methylamino]propanoic acid;
(2R)-2-[[6-[2-chloro-3-[2-chloro-3-[6-methoxy-5-[[[(2R)-5-oxopyrrolidin-2-yl]methylamino] methyl]-2-pyridyl]phenyl]phenyl]-3-methyl-4-oxo-pyrrolo[2,1-f][1,2,4]triazin-2-yl]methylamino]propanoic acid;
(2S)-2-[[6-[2-chloro-3-[2-chloro-3-[6-methoxy-5-[[[(2S)-5-oxopyrrolidin-2-yl]methylamino] methyl]-2-pyridyl]phenyl]phenyl]-3-methyl-4-oxo-pyrrolo[2,1-f][1,2,4]triazin-2-yl]methylamino]propanoic acid;
(2S)-2-[[6-[2-chloro-3-[2-chloro-3-[6-methoxy-5-[[[(2R)-5-oxopyrrolidin-2-yl]methylamino] methyl]-2-pyridyl]phenyl]phenyl]-3-methyl-4-oxo-pyrrolo[2,1-f][1,2,4]triazin-2-yl]methylamino]propanoic acid;
(S)-6-(2,2′-Dichloro-3′-(4-fluoro-6-methoxy-5-((((5-oxopyrrolidin-2-yl)methyl)amino)methyl)pyridin-2-yl)-[1,1′-biphenyl]-3-yl)-2-(((3-fluoropropyl)amino)methyl)-3-methylpyrrolo[2,1-f][1,2,4]triazin-4(3H)-one;
(R)-6-(2,2′-Dichloro-3′-(4-fluoro-6-methoxy-5-((((5-oxopyrrolidin-2-yl)methyl)amino)methyl)pyridin-2-yl)-[1,1′-biphenyl]-3-yl)-2-(((3-fluoropropyl)amino)methyl)-3-methylpyrrolo[2,1-f][1,2,4]triazin-4(3H)-one;
(S)-5-((6-(2,2′-Dichloro-3′-(3-fluoro-6-methoxy-5-(((((S)-5-oxopyrrolidin-2-yl)methyl)amino)methyl)pyridin-2-yl)-[1,1′-biphenyl]-3-yl)-8-methoxy-3,4-dihydroisoquinolin-2(1H)-yl)methyl)pyrrolidin-2-one;
(R)-5-((6-(2,2′-Dichloro-3′-(3-fluoro-6-methoxy-5-(((((S)-5-oxopyrrolidin-2-yl)methyl)amino)methyl)pyridin-2-yl)-[1,1′-biphenyl]-3-yl)-8-methoxy-3,4-dihydroisoquinolin-2(1H)-yl)methyl)pyrrolidin-2-one;
(S)-5-((6-(2,2′-Dichloro-3′-(3-fluoro-6-methoxy-5-(((((R)-5-oxopyrrolidin-2-yl)methyl)amino)methyl)pyridin-2-yl)-[1,1′-biphenyl]-3-yl)-8-methoxy-3,4-dihydroisoquinolin-2(1H)-yl)methyl)pyrrolidin-2-one;
(R)-5-((6-(2,2′-Dichloro-3′-(3-fluoro-6-methoxy-5-(((((R)-5-oxopyrrolidin-2-yl)methyl)amino)methyl)pyridin-2-yl)-[1,1′-biphenyl]-3-yl)-8-methoxy-3,4-dihydroisoquinolin-2(1H)-yl)methyl)pyrrolidin-2-one;
2-(2,2′-Dichloro-3′-(6-methoxy-5-(((((S)-5-oxopyrrolidin-2-yl)methyl)amino)methyl)pyridin-2-yl)-[1,1′-biphenyl]-3-yl)-1-methyl-5-(((((S)-5-oxopyrrolidin-2-yl)methyl)amino)methyl)pyrazolo[1,5-a]pyrimidin-7(1H)-one;
2-(2,2′-Dichloro-3′-(6-methoxy-5-(((((R)-5-oxopyrrolidin-2-yl)methyl)amino)methyl)pyridin-2-yl)-[1,1′-biphenyl]-3-yl)-1-methyl-5-(((((S)-5-oxopyrrolidin-2-yl)methyl)amino)methyl)pyrazolo[1,5-a]pyrimidin-7(1H)-one;
2-(2,2′-Dichloro-3′-(6-methoxy-5-(((((S)-5-oxopyrrolidin-2-yl)methyl)amino)methyl)pyridin-2-yl)-[1,1′-biphenyl]-3-yl)-1-methyl-5-(((((R)-5-oxopyrrolidin-2-yl)methyl)amino)methyl)pyrazolo[1,5-a]pyrimidin-7(1H)-one;
2-(2,2′-Dichloro-3′-(6-methoxy-5-(((((R)-5-oxopyrrolidin-2-yl)methyl)amino)methyl)pyridin-2-yl)-[1,1′-biphenyl]-3-yl)-1-methyl-5-(((((R)-5-oxopyrrolidin-2-yl)methyl)amino)methyl)pyrazolo[1,5-a]pyrimidin-7(1H)-one;
6-(2,2′-Dichloro-3′-(3-fluoro-6-methoxy-5-(((((S)-5-oxopyrrolidin-2-yl)methyl)amino)methyl)pyridin-2-yl)-[1,1′-biphenyl]-3-yl)-2-((((1r,3r)-3-hydroxy-3-methylcyclobutyl)amino)methyl)-3-methylpyrrolo[2,1-f][1,2,4]triazin-4(3H)-one;
6-(2,2′-Dichloro-3′-(3-fluoro-6-methoxy-5-(((((S)-5-oxopyrrolidin-2-yl)methyl)amino)methyl)pyridin-2-yl)-[1,1′-biphenyl]-3-yl)-2-((((1s,3s)-3-hydroxy-3-methylcyclobutyl)amino)methyl)-3-methylpyrrolo[2,1-f][1,2,4]triazin-4(3H)-one;
6-(2,2′-Dichloro-3′-(3-fluoro-6-methoxy-5-(((((R)-5-oxopyrrolidin-2-yl)methyl)amino)methyl)pyridin-2-yl)-[1,1′-biphenyl]-3-yl)-2-((((1r,3r)-3-hydroxy-3-methylcyclobutyl)amino)methyl)-3-methylpyrrolo[2,1-f][1,2,4]triazin-4(3H)-one;
6-(2,2′-Dichloro-3′-(3-fluoro-6-methoxy-5-(((((R)-5-oxopyrrolidin-2-yl)methyl)amino)methyl)pyridin-2-yl)-[1,1′-biphenyl]-3-yl)-2-((((1s,3s)-3-hydroxy-3-methylcyclobutyl)amino)methyl)-3-methylpyrrolo[2,1-f][1,2,4]triazin-4(3H)-one;
(S)-Isopropyl 2-(((6-(2,2′-dichloro-3′-(4-chloro-6-methoxy-5-((((5-oxopyrrolidin-2-yl)methyl)amino)methyl)pyridin-2-yl)-[1,1′-biphenyl]-3-yl)-3-methyl-4-oxo-3,4-dihydropyrrolo[2,1-f][1,2,4]triazin-2-yl)methyl)amino)-2-methylpropanoate;
(R)-Isopropyl 2-(((6-(2,2′-dichloro-3′-(4-chloro-6-methoxy-5-((((5-oxopyrrolidin-2-yl)methyl)amino)methyl)pyridin-2-yl)-[1,1′-biphenyl]-3-yl)-3-methyl-4-oxo-3,4-dihydropyrrolo[2,1-f][1,2,4]triazin-2-yl)methyl)amino)-2-methylpropanoate;
(3S)-1-[[6-[2-Chloro-3-[2-chloro-3-[6-methoxy-5-[[[(2S)-5-oxopyrrolidin-2-yl]methylamino]methyl]-2-pyridyl]phenyl]phenyl]-3-methyl-4-oxo-pyrrolo[2,1-f][1,2,4]triazin-2-yl]methyl]piperidine-3-carboxylic acid;
(3S)-1-[[6-[2-Chloro-3-[2-chloro-3-[6-methoxy-5-[[[(2R)-5-oxopyrrolidin-2-yl]methylamino]methyl]-2-pyridyl]phenyl]phenyl]-3-methyl-4-oxo-pyrrolo[2,1-f][1,2,4]triazin-2-yl]methyl]piperidine-3-carboxylic acid;
(3R)-1-[[6-[2-Chloro-3-[2-chloro-3-[6-methoxy-5-[[[(2S)-5-oxopyrrolidin-2-yl]methylamino]methyl]-2-pyridyl]phenyl]phenyl]-3-methyl-4-oxo-pyrrolo[2,1-f][1,2,4]triazin-2-yl]methyl]piperidine-3-carboxylic acid;
(3R)-1-[[6-[2-Chloro-3-[2-chloro-3-[6-methoxy-5-[[[(2R)-5-oxopyrrolidin-2-yl]methylamino]methyl]-2-pyridyl]phenyl]phenyl]-3-methyl-4-oxo-pyrrolo[2,1-f][1,2,4]triazin-2-yl]methyl]piperidine-3-carboxylic acid;
(S)-2-(2,2′-Dichloro-3′-(6-methoxy-5-((((5-oxopyrrolidin-2-yl)methyl)amino)methyl)pyridin-2-yl)-[1,1′-biphenyl]-3-yl)-5-(((2-hydroxyethyl)amino)methyl)-1-methylpyrazolo[1,5-a]pyrimidin-7(1H)-one;
(R)-2-(2,2′-Dichloro-3′-(6-methoxy-5-((((5-oxopyrrolidin-2-yl)methyl)amino)methyl)pyridin-2-yl)-[1,1′-biphenyl]-3-yl)-5-(((2-hydroxyethyl)amino)methyl)-1-methylpyrazolo[1,5-a]pyrimidin-7(1H)-one;
(5S)-5-[[[6-[3-[3-[2-[[(1-Acetyl-4-piperidyl)amino]methyl]-[1,2,4]triazolo[1,5-a]pyrimidin-6-yl]-2-chloro-phenyl]-2-chloro-phenyl]-2-methoxy-3-pyridyl]methylamino]methyl] pyrrolidin-2-one;
(5R)-5-[[[6-[3-[3-[2-[[(1-Acetyl-4-piperidyl)amino]methyl]-[1,2,4]triazolo[1,5-a]pyrimidin-6-yl]-2-chloro-phenyl]-2-chloro-phenyl]-2-methoxy-3-pyridyl]methylamino]methyl] pyrrolidin-2-one;
3-[[6-[2-Chloro-3-[2-chloro-3-[6-methoxy-5-[[[(2S)-5-oxopyrrolidin-2-yl]methylamino]methyl]-2-pyridyl]phenyl]phenyl]-3-methyl-4-oxo-pyrrolo[2,1-f][1,2,4]triazin-2-yl]methylamino]propanoic acid;
3-[[6-[2-Chloro-3-[2-chloro-3-[6-methoxy-5-[[[(2R)-5-oxopyrrolidin-2-yl]methylamino]methyl]-2-pyridyl]phenyl]phenyl]-3-methyl-4-oxo-pyrrolo[2,1-f][1,2,4]triazin-2-yl]methylamino]propanoic acid;
(S)-2-(2,2′-Dichloro-3′-(6-methoxy-5-((((5-oxopyrrolidin-2-yl)methyl)amino)methyl)pyridin-2-yl)-[1,1′-biphenyl]-3-yl)-5-((6-hydroxy-2-azaspiro[3.3]heptan-2-yl)methyl)-1-methylpyrazolo[1,5-a]pyrimidin-7(1H)-one;
(R)-2-(2,2′-Dichloro-3′-(6-methoxy-5-((((5-oxopyrrolidin-2-yl)methyl)amino)methyl)pyridin-2-yl)-[1,1′-biphenyl]-3-yl)-5-((6-hydroxy-2-azaspiro[3.3]heptan-2-yl)methyl)-1-methylpyrazolo[1,5-a]pyrimidin-7(1H)-one.

The compounds of the invention may possess one or more stereocenters, and each stereocenter may exist independently in either the (R) or (S) configuration. In certain embodiments, compounds described herein are present in optically active or racemic forms.

The compounds described herein encompass racemic, optically active, regioisomeric and stereoisomeric forms, or combinations thereof that possess the therapeutically useful properties described herein. Preparation of optically active forms is achieved in any suitable manner, including by way of non-limiting example, by resolution of the racemic form with recrystallization techniques, synthesis from optically active starting materials, chiral synthesis, or chromatographic separation using a chiral stationary phase. A compound illustrated herein by the racemic formula further represents either of the two enantiomers or mixtures thereof, or in the case where two or more chiral center are present, all diastereomers or mixtures thereof.

In certain embodiments, the compounds of the invention exist as tautomers. All tautomers are included within the scope of the compounds recited herein.

Compounds described herein also include isotopically labeled compounds wherein one or more atoms is replaced by an atom having the same atomic number, but an atomic mass or mass number different from the atomic mass or mass number usually found in nature. Examples of isotopes suitable for inclusion in the compounds described herein include and are not limited to ²H, ³H, ¹¹C, ¹³C, ¹⁴C, ³⁶C₁, ¹⁸F, ¹²³I ¹²⁵I ¹³N, ¹⁵N, ¹⁵O, ¹⁷O ¹⁸O, ³²P, and ³⁵S.

In certain embodiments, substitution with heavier isotopes such as deuterium affords greater chemical stability. Isotopically labeled compounds are prepared by any suitable method or by processes using an appropriate isotopically labeled reagent in place of the non-labeled reagent otherwise employed.

In certain embodiments, the compounds described herein are labeled by other means, including, but not limited to, the use of chromophores or fluorescent moieties, bioluminescent labels, or chemiluminescent labels.

In all of the embodiments provided herein, examples of suitable optional substituents are not intended to limit the scope of the claimed invention. The compounds of the invention may contain any of the substituents, or combinations of substituents, provided herein.

### Salts

The compounds described herein may form salts with acids or bases, and such salts are included in the present invention. The term “salts” embraces addition salts of free acids or bases that are useful within the methods of the invention. The term “pharmaceutically acceptable salt” refers to salts that possess toxicity profiles within a range that affords utility in pharmaceutical applications. In certain embodiments, the salts are pharmaceutically acceptable salts. Pharmaceutically unacceptable salts may nonetheless possess properties such as high crystallinity, which have utility in the practice of the present invention, such as for example utility in process of synthesis, purification or formulation of compounds useful within the methods of the invention.

Suitable pharmaceutically acceptable acid addition salts may be prepared from an inorganic acid or from an organic acid. Examples of inorganic acids include sulfate, hydrogen sulfate, hydrochloric, hydrobromic, hydriodic, nitric, carbonic, sulfuric, and phosphoric acids (including hydrogen phosphate and dihydrogen phosphate). Appropriate organic acids may be selected from aliphatic, cycloaliphatic, aromatic, araliphatic, heterocyclic, carboxylic and sulfonic classes of organic acids, examples of which include formic, acetic, propionic, succinic, glycolic, gluconic, lactic, malic, tartaric, citric, ascorbic, glucuronic, maleic, fumaric, pyruvic, aspartic, glutamic, benzoic, anthranilic, 4-hydroxybenzoic, phenylacetic, mandelic, embonic (or pamoic), methanesulfonic, ethanesulfonic, benzenesulfonic, pantothenic, sulfanilic, 2-hydroxyethanesulfonic, trifluoromethanesulfonic, p-toluenesulfonic, cyclohexylaminosulfonic, stearic, alginic, O-hydroxybutyric, salicylic, galactaric, galacturonic acid, glycerophosphonic acids and saccharin (e.g., saccharinate, saccharate). Salts may be comprised of a fraction of one, one or more than one molar equivalent of acid or base with respect to any compound of the invention.

Suitable pharmaceutically acceptable base addition salts of compounds of the invention include, for example, ammonium salts and metallic salts including alkali metal, alkaline earth metal and transition metal salts such as, for example, calcium, magnesium, potassium, sodium and zinc salts. Pharmaceutically acceptable base addition salts also include organic salts made from basic amines such as, for example, N,N′-dibenzylethylene-diamine, chloroprocaine, choline, diethanolamine, ethylenediamine, meglumine (or N-methylglucamine) and procaine. All of these salts may be prepared from the corresponding compound by reacting, for example, the appropriate acid or base with the compound.

Combination Therapies

In one aspect, the compounds of the invention are useful within the methods of the invention in combination with one or more additional agents useful for treating HBV and/or HDV infections. These additional agents may comprise compounds or compositions identified herein, or compounds (e.g., commercially available compounds) known to treat, prevent, or reduce the symptoms of HBV and/or HDV infections.

Non-limiting examples of one or more additional agents useful for treating HBV and/or HDV infections include: (a) reverse transcriptase inhibitors; (b) capsid inhibitors; (c) cccDNA formation inhibitors; (d) RNA destabilizers; (e) oligomeric nucleotides targeted against the HBV genome; (f) immunostimulators, such as checkpoint inhibitors (e.g., PD-L1 inhibitors); and (g) GalNAc-siRNA conjugates targeted against an HBV gene transcript.

(a) Reverse Transcriptase Inhibitors

In certain embodiments, the reverse transcriptase inhibitor is a reverse-transcriptase inhibitor (NARTI or NRTI). In other embodiments, the reverse transcriptase inhibitor is a nucleotide analog reverse-transcriptase inhibitor (NtARTI or NtRTI).

Reported reverse transcriptase inhibitors include, but are not limited to, entecavir, clevudine, telbivudine, lamivudine, adefovir, and tenofovir, tenofovir disoproxil, tenofovir alafenamide, adefovir dipovoxil, (1R,2R,3R,5R)-3-(6-amino-9H-9-purinyl)-2-fluoro-5-(hydroxymethyl)-4-methylenecyclopentan-1-ol (described in U.S. Pat. No. 8,816,074, incorporated herein in its entirety by reference), emtricitabine, abacavir, elvucitabine, ganciclovir, lobucavir, famciclovir, penciclovir, and amdoxovir.

Reported reverse transcriptase inhibitors further include, but are not limited to, entecavir, lamivudine, and (1R,2R,3R,5R)-3-(6-amino-9H-9-purinyl)-2-fluoro-5-(hydroxymethyl)-4-methylenecyclopentan-1-ol.

Reported reverse transcriptase inhibitors further include, but are not limited to, a covalently bound phosphoramidate or phosphonamidate moiety of the above-mentioned reverse transcriptase inhibitors, or as described in for example U.S. Pat. No. 8,816,074, US Patent Application Publications No. US 2011/0245484 A1, and US 2008/0286230A1, all of which incorporated herein in their entireties by reference.

Reported reverse transcriptase inhibitors further include, but are not limited to, nucleotide analogs that comprise a phosphoramidate moiety, such as, for example, methyl ((((1R,3R,4R,5R)-3-(6-amino-9H-purin-9-yl)-4-fluoro-5-hydroxy-2-methylenecyclopentyl) methoxy)(phenoxy) phosphoryl)-(D or L)-alaninate and methyl ((((1R,2R,3R,4R)-3-fluoro-2-hydroxy-5-methylene-4-(6-oxo-1,6-dihydro-9H-purin-9-yl)cyclopentyl)methoxy)(phenoxy) phosphoryl)-(D or L)-alaninate. Also included are the individual diastereomers thereof, which include, for example, methyl ((R)-(((1R,3R,4R,5R)-3-(6-amino-9H-purin-9-yl)-4-fluoro-5-hydroxy-2-methylenecyclopentyl)methoxy)(phenoxy)phosphoryl)-(D or L)-alaninate and methyl ((S)-(((1R,3R,4R,5R)-3-(6-amino-9H-purin-9-yl)-4-fluoro-5-hydroxy-2-methylenecyclopentyl) methoxy)(phenoxy)phosphoryl)-(D or L)-alaninate.

Reported reverse transcriptase inhibitors further include, but are not limited to, compounds comprising a phosphonamidate moiety, such as, for example, tenofovir alafenamide, as well as those described in U.S. Patent Application Publication No. US 2008/0286230 A1, incorporated herein in its entirety by reference. Methods for preparing stereoselective phosphoramidate or phosphonamidate containing actives are described in, for example, U.S. Pat. No. 8,816,074, as well as U.S. Patent Application Publications No. US 2011/0245484 A1 and US 2008/0286230 A1, all of which incorporated herein in their entireties by reference.

(b) Capsid Inhibitors

As described herein, the term “capsid inhibitor” includes compounds that are capable of inhibiting the expression and/or function of a capsid protein either directly or indirectly.

For example, a capsid inhibitor may include, but is not limited to, any compound that inhibits capsid assembly, induces formation of non-capsid polymers, promotes excess capsid assembly or misdirected capsid assembly, affects capsid stabilization, and/or inhibits encapsidation of RNA (pgRNA). Capsid inhibitors also include any compound that inhibits capsid function in a downstream event(s) within the replication process (e.g., viral DNA synthesis, transport of relaxed circular DNA (rcDNA) into the nucleus, covalently closed circular DNA (cccDNA) formation, virus maturation, budding and/or release, and the like). For example, in certain embodiments, the inhibitor detectably inhibits the expression level or biological activity of the capsid protein as measured, e.g., using an assay described herein. In certain embodiments, the inhibitor inhibits the level of rcDNA and downstream products of viral life cycle by at least 5%, at least 10%, at least 20%, at least 50%, at least 75%, or at least 90%.

Reported capsid inhibitors include, but are not limited to, compounds described in International Patent Applications Publication Nos WO 2013006394, WO 2014106019, and WO2014089296, all of which incorporated herein in their entireties by reference.

Reported capsid inhibitors also include, but are not limited to, the following compounds and pharmaceutically acceptable salts and/or solvates thereof: Bay-41-4109 (see Int'l Patent Application Publication No. WO 2013144129), AT-61 (see Int'l Patent Application Publication No. WO 1998033501; and King, et al., 1998, Antimicrob. Agents Chemother. 42(12):3179-3186), DVR-01 and DVR-23 (see Int'l Patent Application Publication No. WO 2013006394; and Campagna, et al., 2013, J. Virol. 87(12):6931, all of which incorporated herein in their entireties by reference.

In addition, reported capsid inhibitors include, but are not limited to, those generally and specifically described in U.S. Patent Application Publication Nos. US 2015/0225355, US 2015/0132258, US 2016/0083383, US 2016/0052921, US 2019/0225593, and Int'l Patent Application Publication Nos. WO 2013096744, WO 2014165128, WO 2014033170, WO 2014033167, WO 2014033176, WO 2014131847, WO 2014161888, WO 2014184350, WO 2014184365, WO 2015059212, WO 2015011281, WO 2015118057, WO 2015109130, WO 2015073774, WO 2015180631, WO 2015138895, WO 2016089990, WO 2017015451, WO 2016183266, WO 2017011552, WO 2017048950, WO2017048954, WO 2017048962, WO 2017064156, WO 2018052967, WO 2018172852, WO 2020023710, WO2020123674, and are incorporated herein in their entirety by reference.

(c) cccDNA Formation Inhibitors

Covalently closed circular DNA (cccDNA) is generated in the cell nucleus from viral rcDNA and serves as the transcription template for viral mRNAs. As described herein, the term “cccDNA formation inhibitor” includes compounds that are capable of inhibiting the formation and/or stability of cccDNA either directly or indirectly. For example, a cccDNA formation inhibitor may include, but is not limited to, any compound that inhibits capsid disassembly, rcDNA entry into the nucleus, and/or the conversion of rcDNA into cccDNA.

For example, in certain embodiments, the inhibitor detectably inhibits the formation and/or stability of the cccDNA as measured, e.g., using an assay described herein. In certain embodiments, the inhibitor inhibits the formation and/or stability of cccDNA by at least 5%, at least 10%, at least 20%, at least 50%, at least 75%, or at least 90%.

Reported cccDNA formation inhibitors include, but are not limited to, compounds described in Int'l Patent Application Publication No. WO 2013130703, and are incorporated herein in their entirety by reference.

In addition, reported cccDNA formation inhibitors include, but are not limited to, those generally and specifically described in U.S. Patent Application Publication No. US 2015/0038515 A1, and are incorporated herein in their entirety by reference.

(d) RNA Destabilizer

As used herein, the term “RNA destabilizer” refers to a molecule, or a salt or solvate thereof, that reduces the total amount of HBV RNA in mammalian cell culture or in a live human subject. In a non-limiting example, an RNA destabilizer reduces the amount of the RNA transcript(s) encoding one or more of the following HBV proteins: surface antigen, core protein, RNA polymerase, and e antigen. In certain embodiments, the RNA destabilizer reduces the total amount of HBV RNA in mammalian cell culture or in a live human subject by at least 5%, at least 10%, at least 20%, at least 50%, at least 75%, or at least 90%.

Reported RNA destabilizers include compounds described in U.S. Pat. No. 8,921,381, as well as compounds described in U.S. Patent Application Publication Nos. US 2015/0087659 and US 2013/0303552, all of which are incorporated herein in their entireties by reference.

In addition, reported RNA destabilizers include, but are not limited to, those generally and specifically described in Int'l Patent Application Publication Nos. WO 2015113990, WO 2015173164, US 2016/0122344, WO 2016107832, WO 2016023877, WO 2016128335, WO 2016177655, WO 2016071215, WO 2017013046, WO 2017016921, WO 2017016960, WO 2017017042, WO 2017017043, WO 2017102648, WO 2017108630, WO 2017114812, WO 2017140821, WO 2018085619, and are incorporated herein in their entirety by reference.

(e) Oligomeric Nucleotides Targeted Against the HBV Genome Reported oligomeric nucleotides targeted against the HBV genome include, but are not limited to, Arrowhead-ARC-520 (see U.S. Pat. No. 8,809,293; and Wooddell et al., 2013, Molecular Therapy 21(5):973-985, all of which incorporated herein in their entireties by reference).

In certain embodiments, the oligomeric nucleotides can be designed to target one or more genes and/or transcripts of the HBV genome. Oligomeric nucleotide targeted to the HBV genome also include, but are not limited to, isolated, double stranded, siRNA molecules, that each include a sense strand and an antisense strand that is hybridized to the sense strand. In certain embodiments, the siRNA target one or more genes and/or transcripts of the HBV genome.

(f) Immunostimulators

Checkpoint Inhibitors

As described herein, the term “checkpoint inhibitor” includes any compound that is capable of inhibiting immune checkpoint molecules that are regulators of the immune system (e.g., stimulate or inhibit immune system activity). For example, some checkpoint inhibitors block inhibitory checkpoint molecules, thereby stimulating immune system function, such as stimulation of T cell activity against cancer cells. A non-limiting example of a checkpoint inhibitor is a PD-L1 inhibitor.

As described herein, the term “PD-L1 inhibitor” includes any compound that is capable of inhibiting the expression and/or function of the protein Programmed Death-Ligand 1 (PD-L1) either directly or indirectly. PD-L1, also known as cluster of differentiation 274 (CD274) or B7 homolog 1 (B7-H1), is a type 1 transmembrane protein that plays a major role in suppressing the adaptive arm of immune system during pregnancy, tissue allograft transplants, autoimmune disease, and hepatitis. PD-L1 binds to its receptor, the inhibitory checkpoint molecule PD-1 (which is found on activated T cells, B cells, and myeloid cells) so as to modulate activation or inhibition of the adaptive arm of immune system. In certain embodiments, the PD-L1 inhibitor inhibits the expression and/or function of PD-L1 by at least 5%, at least 10%, at least 20%, at least 50%, at least 75%, or at least 90%.

Reported PD-L1 inhibitors include, but are not limited to, compounds recited in one of the following patent application publications: US 2018/0057455; US 2018/0057486; WO 2017/106634; WO 2018/026971; WO 2018/045142; WO 2018/118848; WO 2018/119221; WO 2018/119236; WO 2018/119266; WO 2018/119286; WO 2018/121560; WO 2019/076343; WO 2019/087214; and are incorporated herein in their entirety by reference.

(g) GalNAc-siRNA Conjugates Targeted Against an HBV Gene Transcript

“GalNAc” is the abbreviation for N-acetylgalactosamine, and “siRNA” is the abbreviation for small interfering RNA. An siRNA that targets an HBV gene transcript is covalently bonded to GalNAc in a GalNAc-siRNA conjugate useful in the practice of the present invention. While not wishing to be bound by theory, it is believed that GalNAc binds to asialoglycoprotein receptors on hepatocytes thereby facilitating the targeting of the siRNA to the hepatocytes that are infected with HBV. The siRNA enter the infected hepatocytes and stimulate destruction of HBV gene transcripts by the phenomenon of RNA interference.

Examples of GalNAc-siRNA conjugates useful in the practice of this aspect of the present invention are set forth in published international application PCT/CA2017/050447 (PCT Application Publication number WO/2017/177326, published on Oct. 19, 2017) which is hereby incorporated by reference in its entirety.

A synergistic effect may be calculated, for example, using suitable methods such as, for example, the Sigmoid-Eₘₐₓ equation (Holford & Scheiner, 1981, Clin. Pharmacokinet. 6:429-453), the equation of Loewe additivity (Loewe & Muischnek, 1926, Arch. Exp. Pathol Pharmacol. 114: 313-326) and the median-effect equation (Chou & Talalay, 1984, Adv. Enzyme Regul. 22:27-55). Each equation referred to elsewhere herein may be applied to experimental data to generate a corresponding graph to aid in assessing the effects of the drug combination. The corresponding graphs associated with the equations referred to elsewhere herein are the concentration-effect curve, isobologram curve and combination index curve, respectively.

### Synthesis

The present invention further provides methods of preparing the compounds of the present invention. Compounds of the present teachings can be prepared in accordance with the procedures outlined herein, from commercially available starting materials, compounds known in the literature, or readily prepared intermediates, by employing standard synthetic methods and procedures known to those skilled in the art. Standard synthetic methods and procedures for the preparation of organic molecules and functional group transformations and manipulations can be readily obtained from the relevant scientific literature or from standard textbooks in the field.

It is appreciated that where typical or preferred process conditions (i.e., reaction temperatures, times, mole ratios of reactants, solvents, pressures, and so forth) are given, other process conditions can also be used unless otherwise stated. Optimum reaction conditions can vary with the particular reactants or solvent used, but such conditions can be determined by one skilled in the art by routine optimization procedures. Those skilled in the art of organic synthesis will recognize that the nature and order of the synthetic steps presented can be varied for the purpose of optimizing the formation of the compounds described herein.

The processes described herein can be monitored according to any suitable method known in the art. For example, product formation can be monitored by spectroscopic means, such as nuclear magnetic resonance spectroscopy (e.g., ¹H or ¹³C), infrared spectroscopy, spectrophotometry (e.g., UV-visible), mass spectrometry, or by chromatography such as high pressure liquid chromatograpy (HPLC), gas chromatography (GC), gel-permeation chromatography (GPC), or thin layer chromatography (TLC).

Preparation of the compounds can involve protection and deprotection of various chemical groups. The need for protection and deprotection and the selection of appropriate protecting groups can be readily determined by one skilled in the art. The chemistry of protecting groups can be found, for example, in Greene, et al., Protective Groups in Organic Synthesis, 2d. Ed. (Wiley & Sons, 1991), the entire disclosure of which is incorporated by reference herein for all purposes.

The reactions or the processes described herein can be carried out in suitable solvents that can be readily selected by one skilled in the art of organic synthesis. Suitable solvents typically are substantially nonreactive with the reactants, intermediates, and/or products at the temperatures at which the reactions are carried out, i.e., temperatures that can range from the solvent's freezing temperature to the solvent's boiling temperature. A given reaction can be carried out in one solvent or a mixture of more than one solvent. Depending on the particular reaction step, suitable solvents for a particular reaction step can be selected.

A compound of formula I can be prepared, for example, according to the synthetic methods outlined in Schemes 1-14:

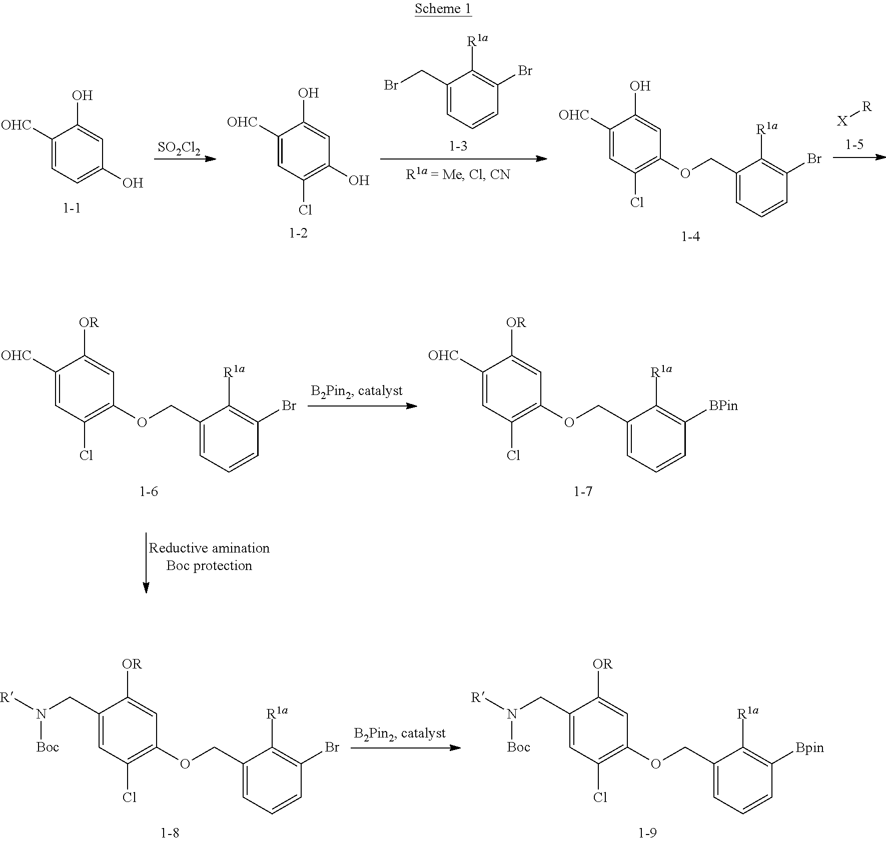

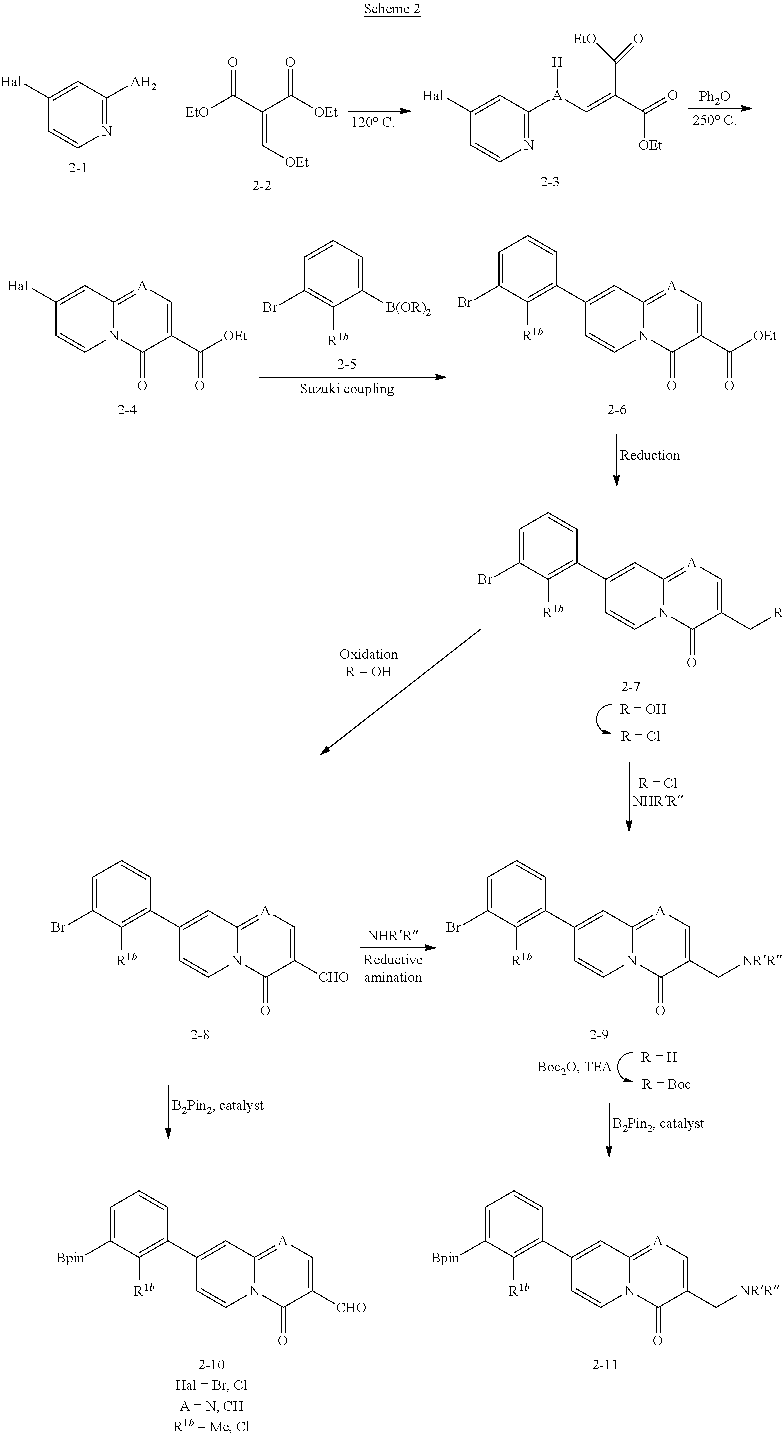

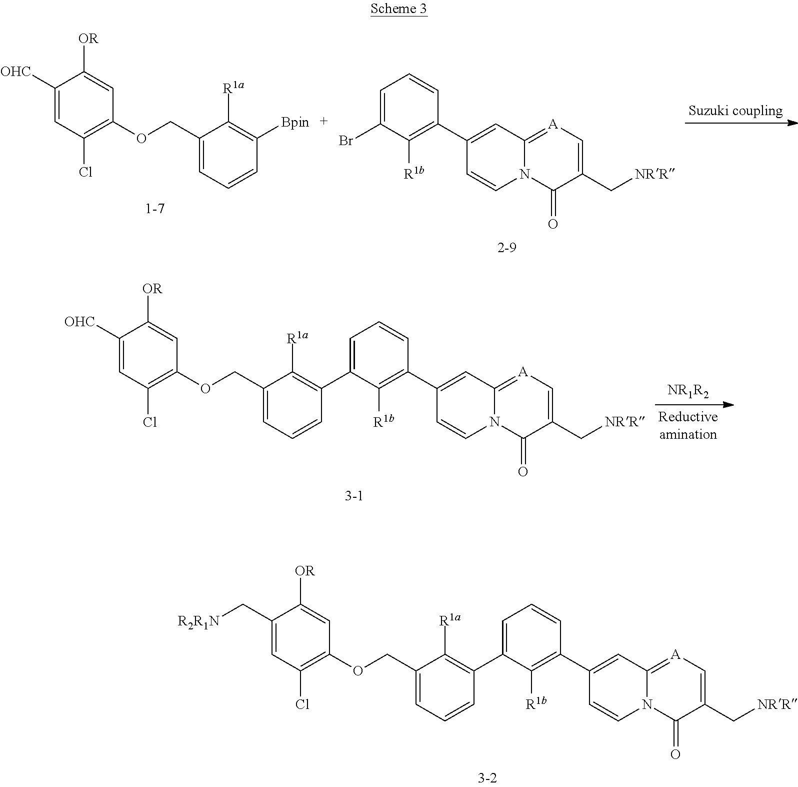

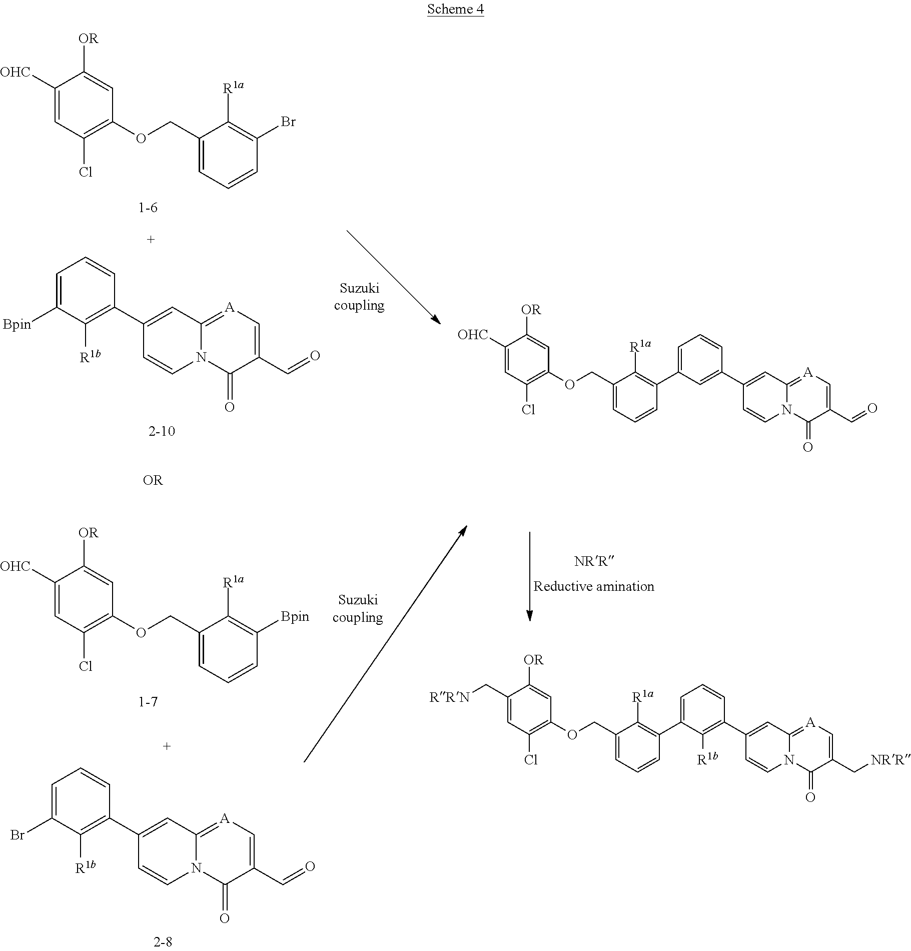

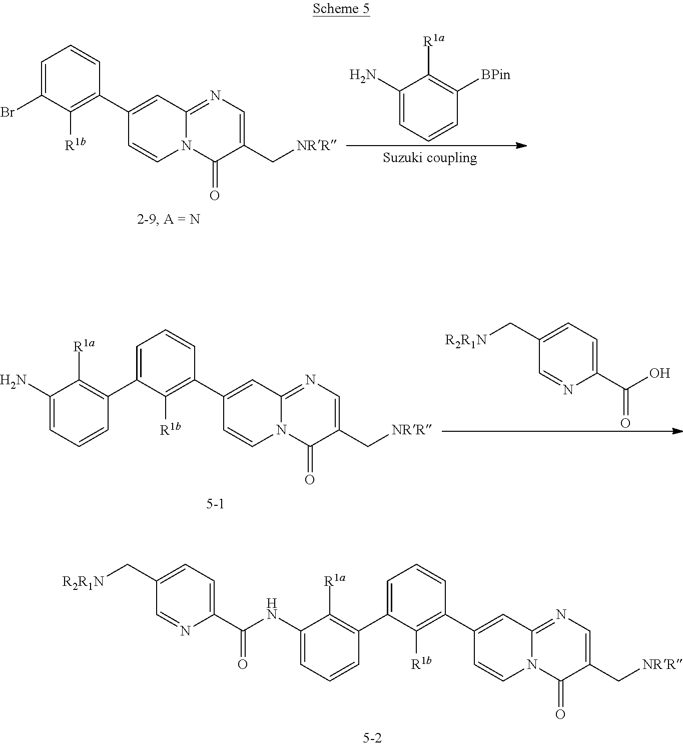

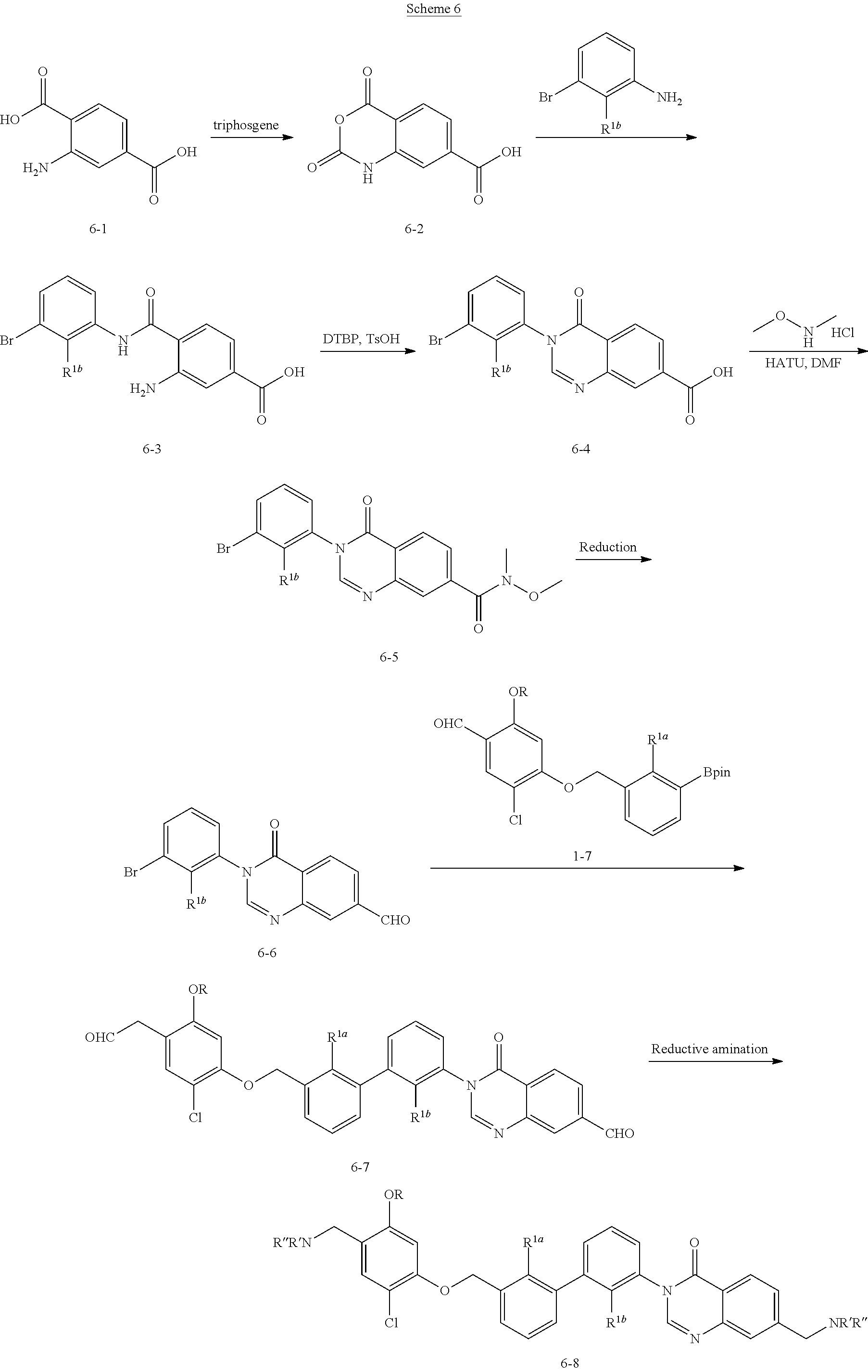

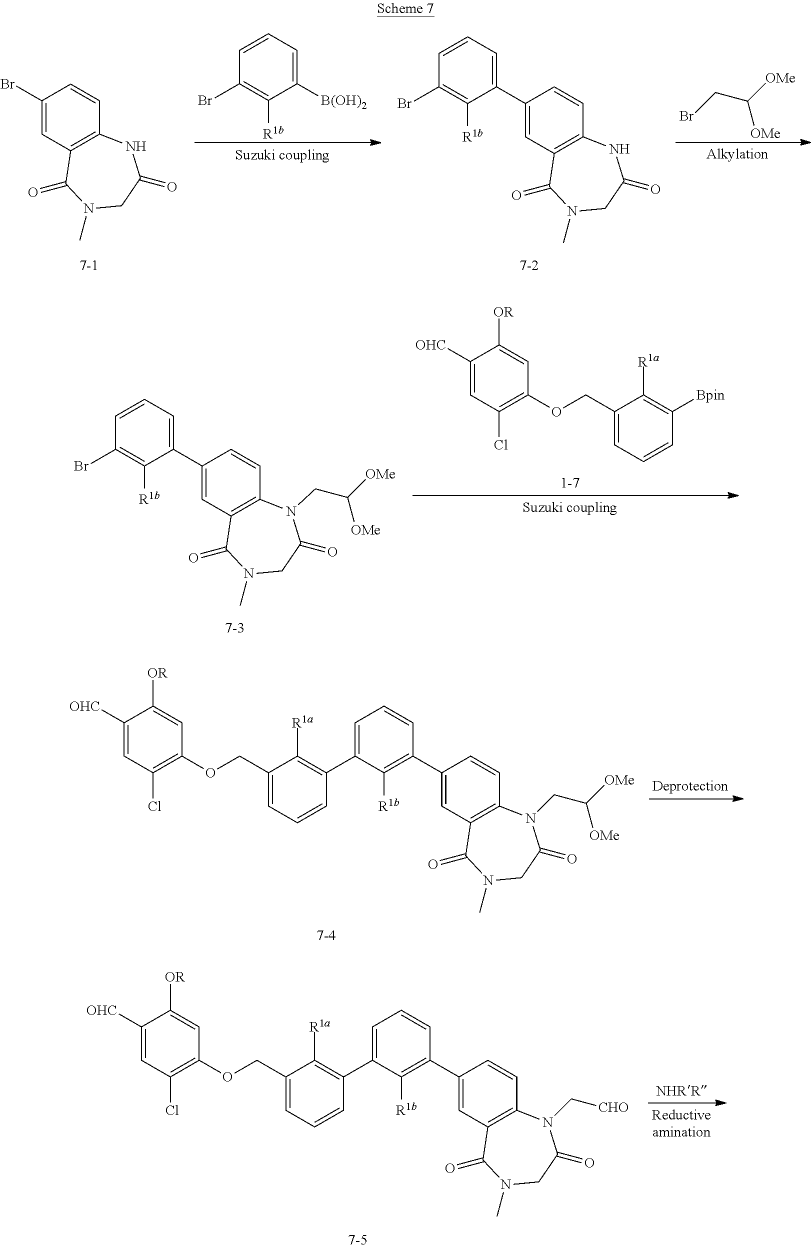

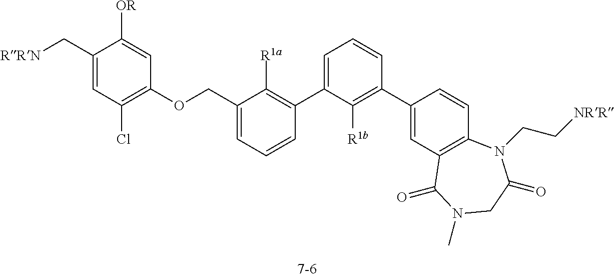

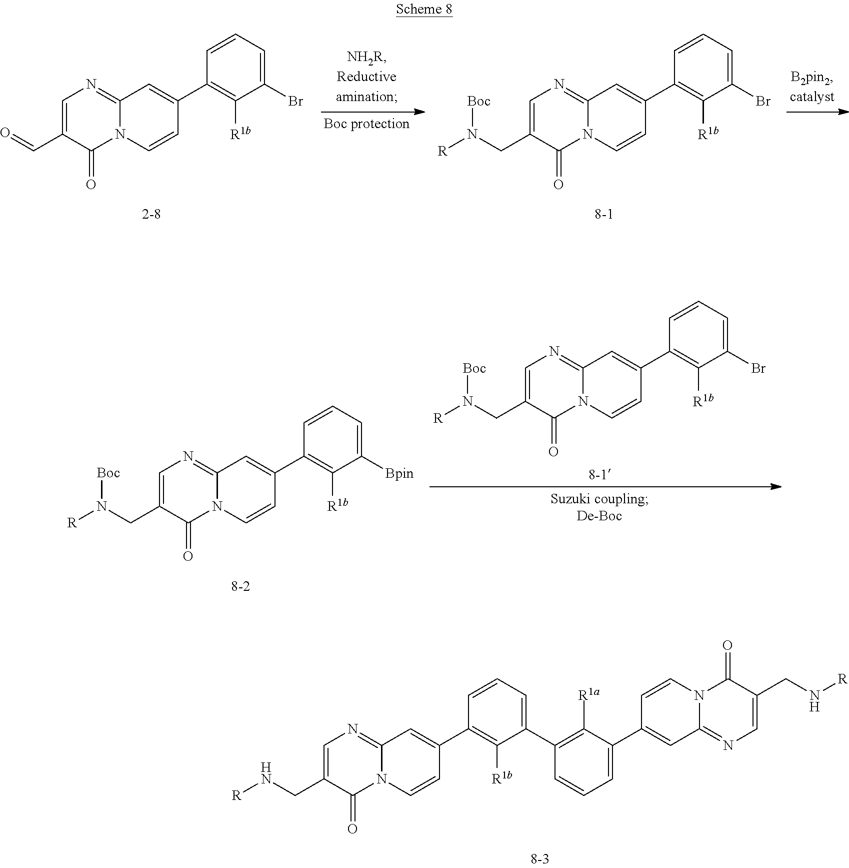

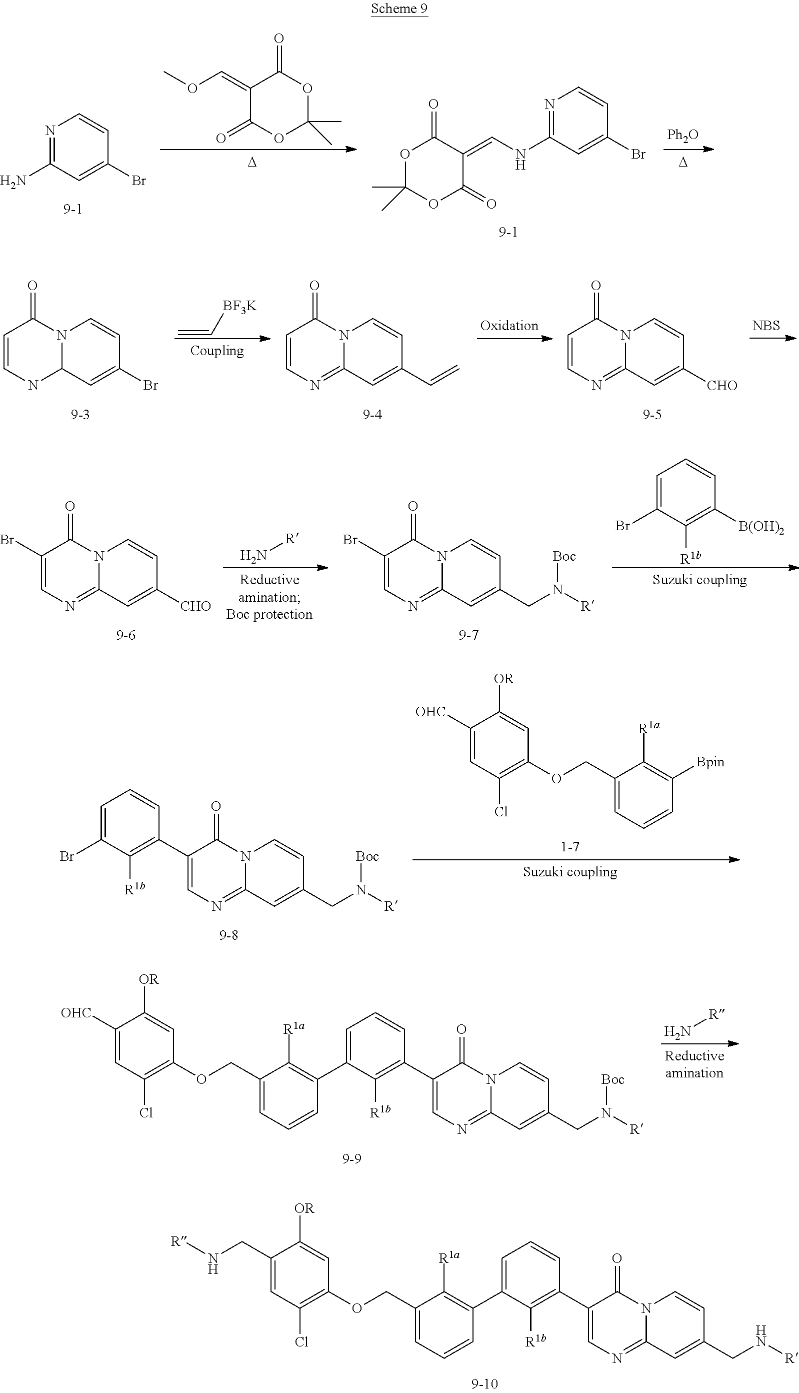

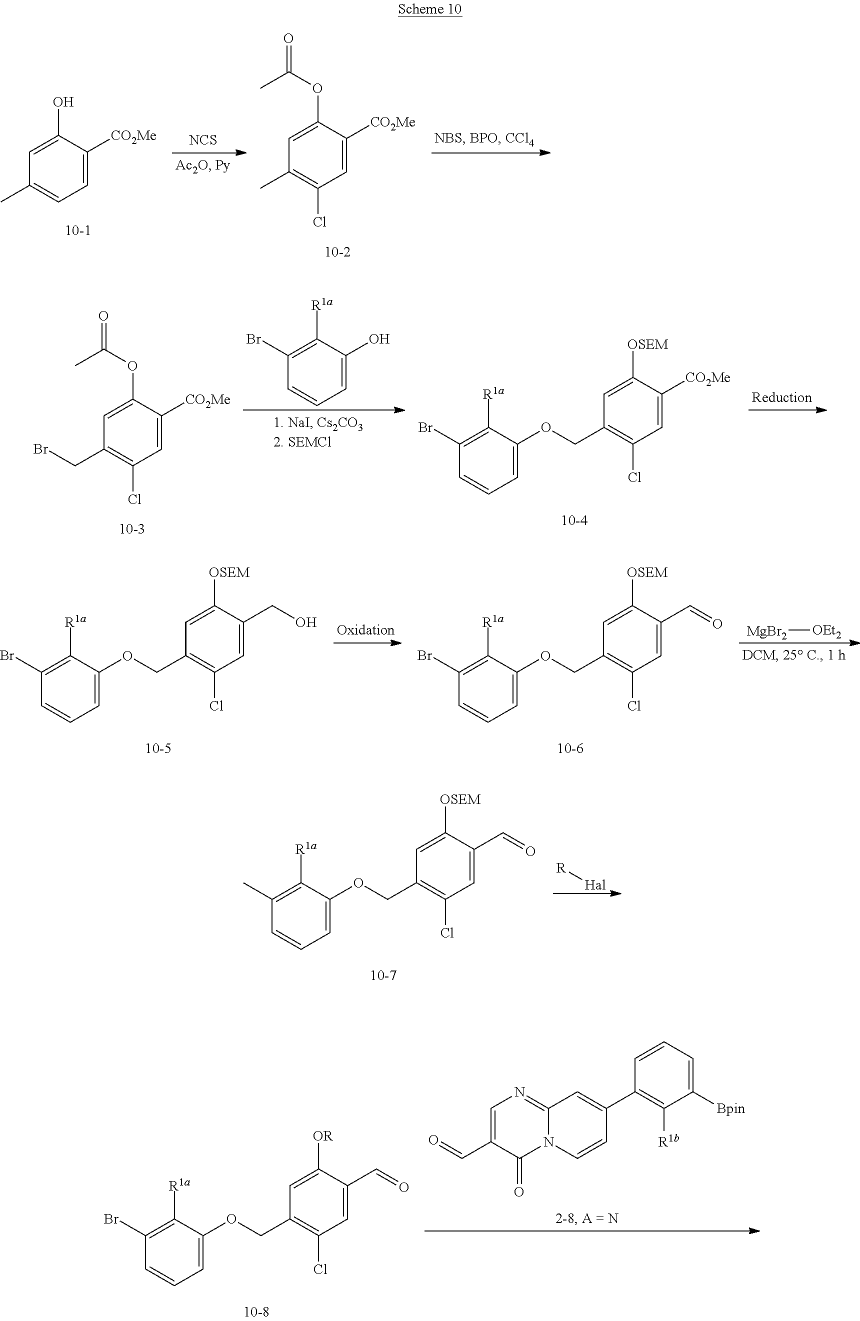

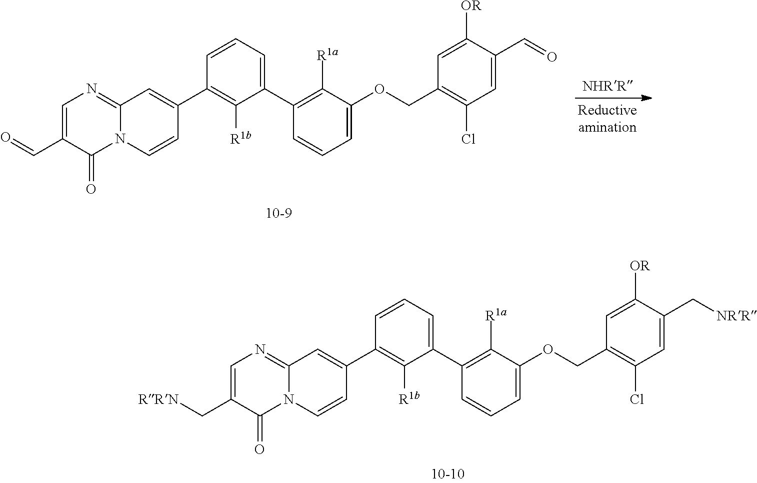

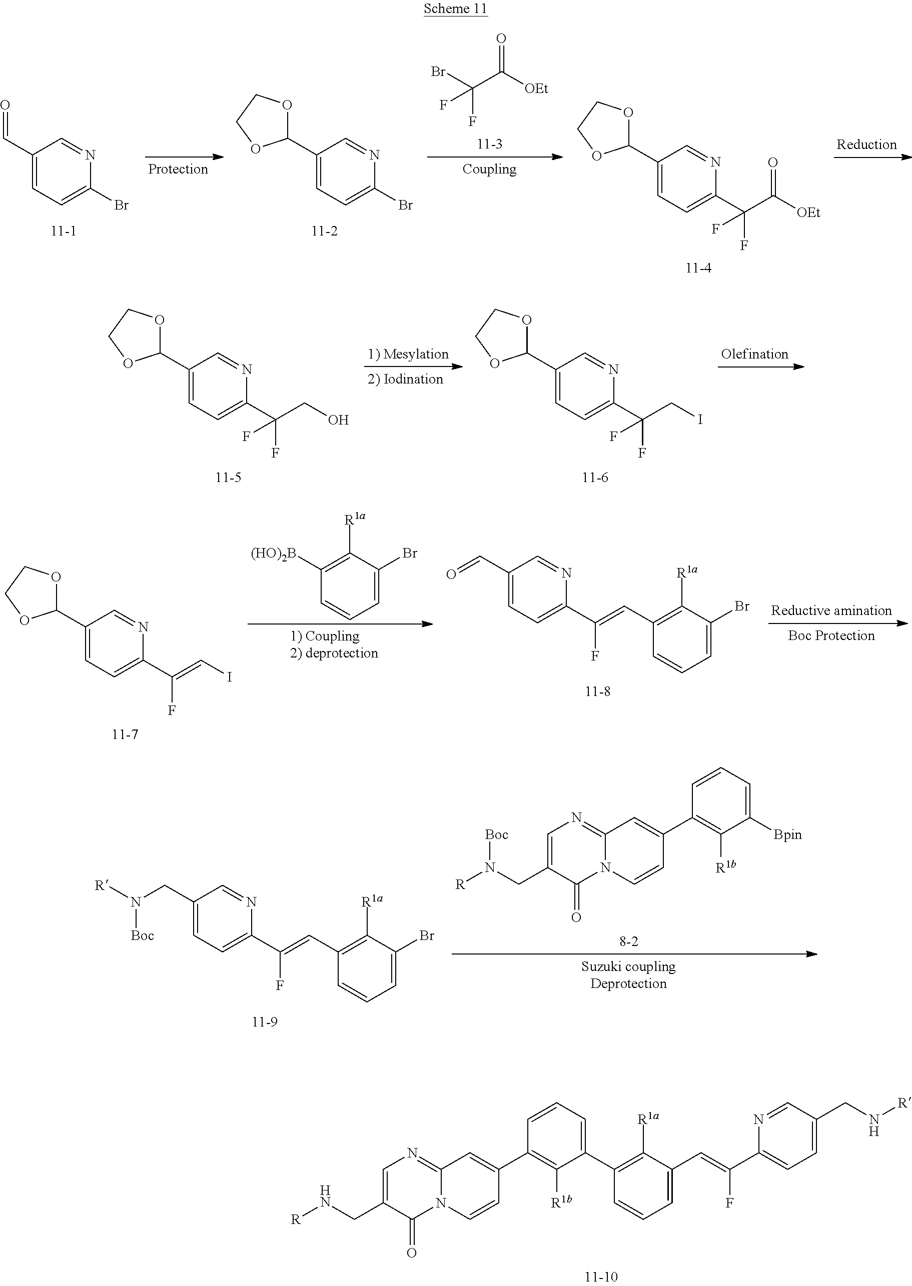

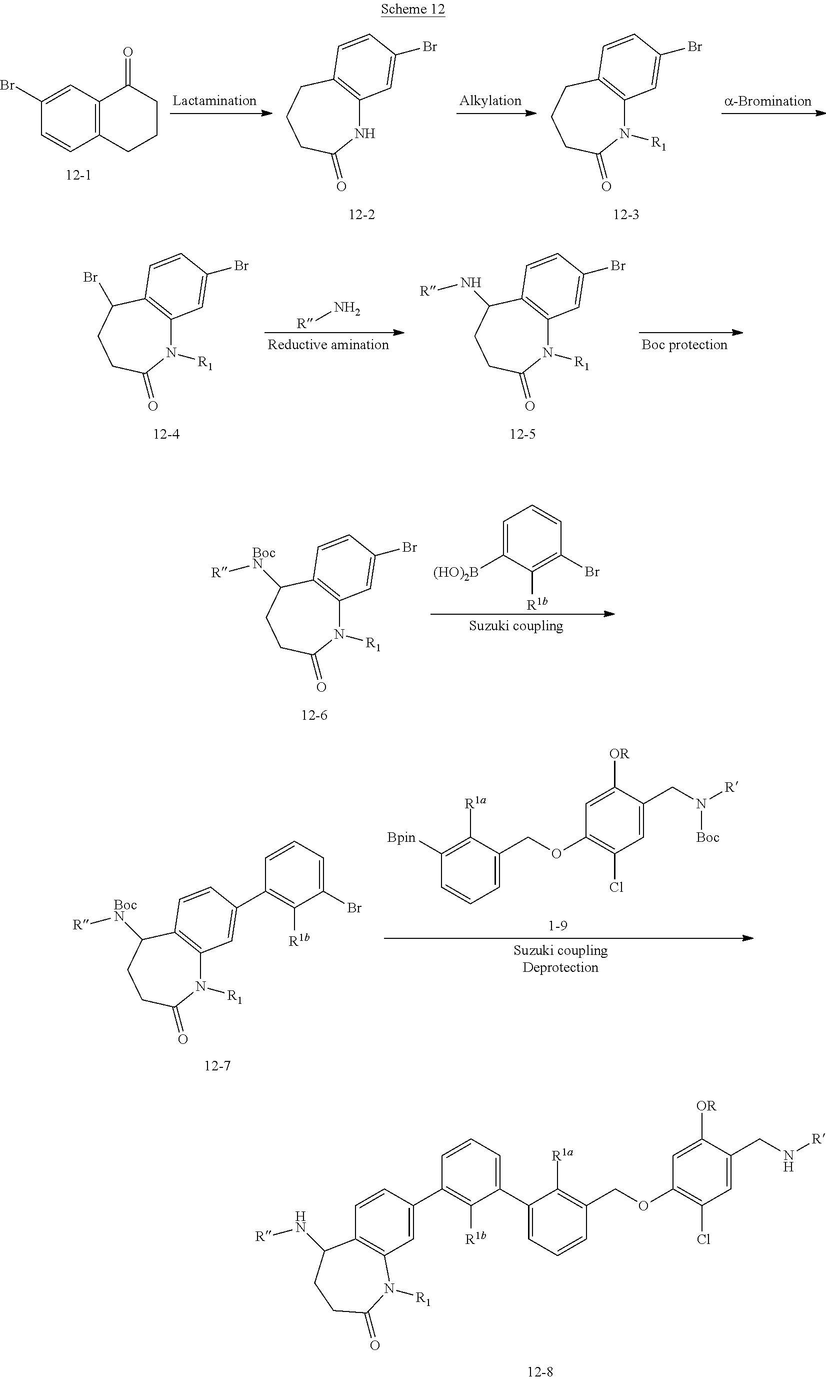

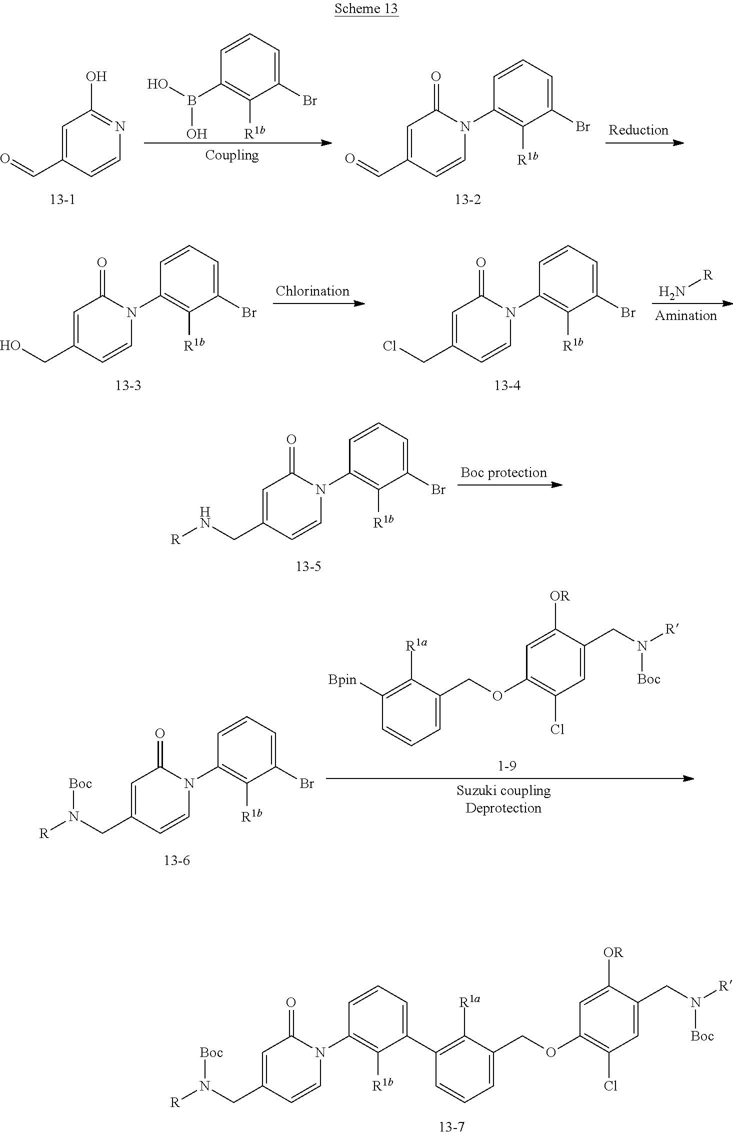

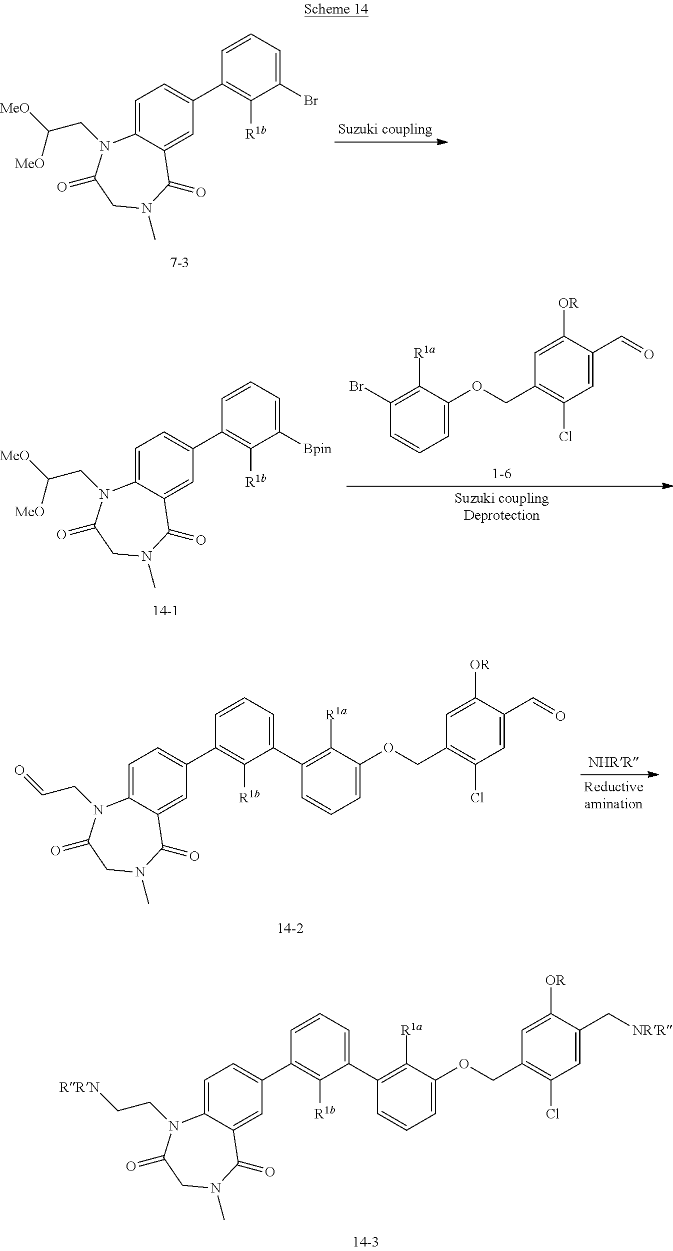

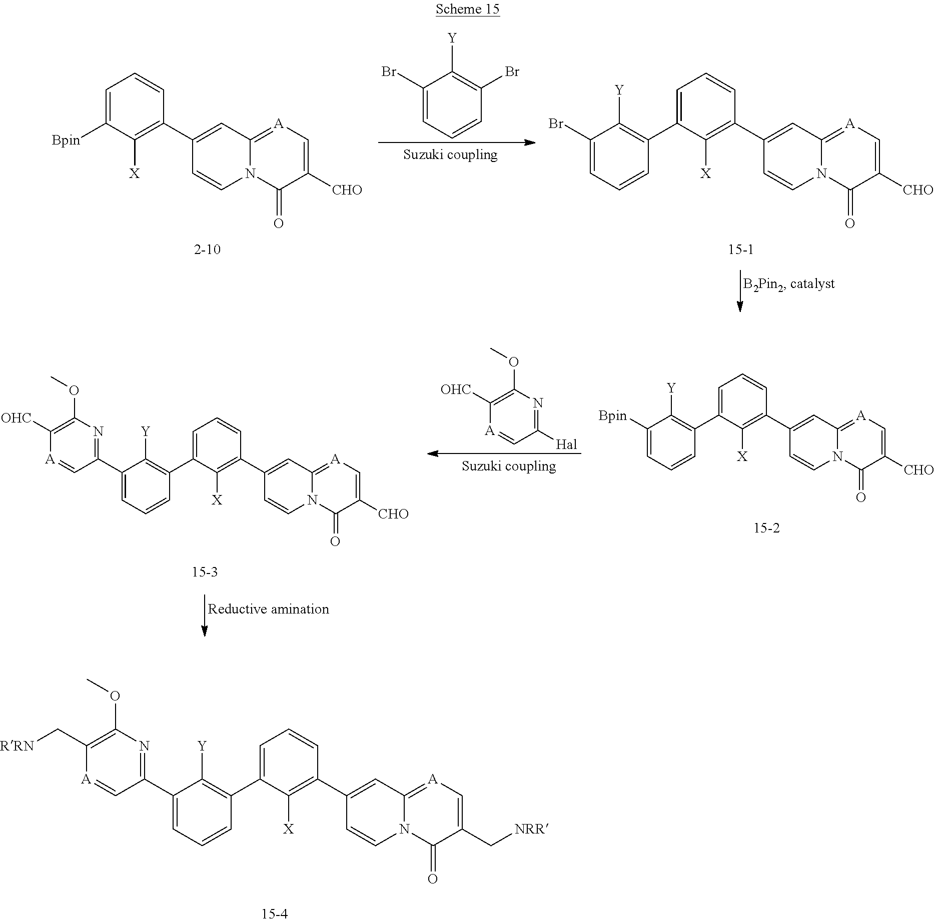

### Methods

The invention provides a method of treating, ameliorating, and/or preventing hepatitis virus infection in a subject. In certain embodiments, the infection comprises hepatitis B virus (HBV) and/or hepatitis D virus (HDV) infection. In other embodiments, the infection comprises hepatitis B virus (HBV) infection. In yet other embodiments, the infection comprises hepatitis D virus (HDV) infection. In yet other embodiments, the method comprises administering to the subject in need thereof a therapeutically effective amount of at least one compound of the invention. In yet other embodiments, the compound of the invention is the only antiviral agent administered to the subject. In yet other embodiments, the at least one compound is administered to the subject in a pharmaceutically acceptable composition. In yet other embodiments, the subject is further administered at least one additional agent useful for treating the hepatitis virus infection. In yet other embodiments, the at least one additional agent comprises at least one selected from the group consisting of reverse transcriptase inhibitors; capsid inhibitors; cccDNA formation inhibitors; RNA destabilizers; oligomeric nucleotides targeted against the HBV genome; immunostimulators; and GalNAc-siRNA conjugates targeted against an HBV gene transcript. In yet other embodiments, the subject is co-administered the at least one compound and the at least one additional agent. In yet other embodiments, the at least one compound and the at least one additional agent are coformulated.

The invention further provides a method of treating, ameliorating, and/or preventing cancer in a subject. In certain embodiments, the method comprises administering to the subject in need thereof a therapeutically effective amount of at least one compound of the invention. In other embodiments, the compound of the invention is the only anticancer agent administered to the subject. In yet other embodiments, the at least one compound is administered to the subject in a pharmaceutically acceptable composition. In yet other embodiments, the subject is further administered at least one additional agent or therapy useful for treating, ameliorating, or preventing the cancer. In yet other embodiments, the additional anticancer agent or therapy comprises nivolumab, pembrolizumab, atezolizumab, ipilimumab, chemotherapy, radiation therapy, and/or resection therapy. In yet other embodiments, the additional anticancer agent or therapy comprises rituxan, doxorubicin, gemcitabine, nivolumab, pembrolizumab, and/or ipilimumab.

In certain embodiments, the cancer is amenable to treatment by inhibiting PD-1, PD-L1 or the PD-1/PD-L1 interaction. In other embodiments, the cancer is at least one of pancreatic cancer, bladder cancer, colorectal cancer, breast cancer, prostate cancer, renal cancer, hepatocellular cancer, lung cancer, ovarian cancer, cervical cancer, gastric cancer, esophageal cancer, head and neck cancer, melanoma, neuroendocrine cancer, CNS cancer, brain cancer, bone cancer, soft tissue sarcoma, non-small cell lung cancer, small-cell lung cancer, or colon cancer. In yet other embodiments, the cancer is at least one of lymphoma, multiple myeloma, or leukemia. In yet other embodiments, the cancer is at least one of acute lymphocytic leukemia (ALL), acute myeloid leukemia (AML), chronic lymphocytic leukemia (CLL), small lymphocytic lymphoma (SLL), myelodysplastic syndrome (MDS), myeloproliferative disease (MPD), chronic myeloid leukemia (CMIL), multiple myeloma (MM), non-Hodgkin's lymphoma (NHL), mantle cell lymphoma (MCL), follicular lymphoma, Waldestrom's macroglobulinemia (WM), T-cell lymphoma, B-cell lymphoma and diffuse large B-cell lymphoma (DLBCL).

In certain embodiments, the subject is a mammal. In other embodiments, the mammal is a human.

### Pharmaceutical Compositions and Formulations

The invention provides pharmaceutical compositions comprising at least one compound of the invention or a salt or solvate thereof, which are useful to practice methods of the invention. Such a pharmaceutical composition may consist of at least one compound of the invention or a salt or solvate thereof, in a form suitable for administration to a subject, or the pharmaceutical composition may comprise at least one compound of the invention or a salt or solvate thereof, and one or more pharmaceutically acceptable carriers, one or more additional ingredients, or some combination of these. At least one compound of the invention may be present in the pharmaceutical composition in the form of a physiologically acceptable salt, such as in combination with a physiologically acceptable cation or anion, as is well known in the art.

In certain embodiments, the pharmaceutical compositions useful for practicing the method of the invention may be administered to deliver a dose of between 1 ng/kg/day and 100 mg/kg/day. In other embodiments, the pharmaceutical compositions useful for practicing the invention may be administered to deliver a dose of between 1 ng/kg/day and 1,000 mg/kg/day.

The relative amounts of the active ingredient, the pharmaceutically acceptable carrier, and any additional ingredients in a pharmaceutical composition of the invention will vary, depending upon the identity, size, and condition of the subject treated and further depending upon the route by which the composition is to be administered. By way of example, the composition may comprise between 0.1% and 100% (w/w) active ingredient.

Pharmaceutical compositions that are useful in the methods of the invention may be suitably developed for nasal, inhalational, oral, rectal, vaginal, pleural, peritoneal, parenteral, topical, transdermal, pulmonary, intranasal, buccal, ophthalmic, epidural, intrathecal, intravenous or another route of administration. A composition useful within the methods of the invention may be directly administered to the brain, the brainstem, or any other part of the central nervous system of a mammal or bird. Other contemplated formulations include projected nanoparticles, microspheres, liposomal preparations, coated particles, polymer conjugates, resealed erythrocytes containing the active ingredient, and immunologically-based formulations.

In certain embodiments, the compositions of the invention are part of a pharmaceutical matrix, which allows for manipulation of insoluble materials and improvement of the bioavailability thereof, development of controlled or sustained release products, and generation of homogeneous compositions. By way of example, a pharmaceutical matrix may be prepared using hot melt extrusion, solid solutions, solid dispersions, size reduction technologies, molecular complexes (e.g., cyclodextrins, and others), microparticulate, and particle and formulation coating processes. Amorphous or crystalline phases may be used in such processes.

The route(s) of administration will be readily apparent to the skilled artisan and will depend upon any number of factors including the type and severity of the disease being treated, the type and age of the veterinary or human patient being treated, and the like.

The formulations of the pharmaceutical compositions described herein may be prepared by any method known or hereafter developed in the art of pharmacology and pharmaceutics. In general, such preparatory methods include the step of bringing the active ingredient into association with a carrier or one or more other accessory ingredients, and then, if necessary or desirable, shaping or packaging the product into a desired single-dose or multi-dose unit.

As used herein, a “unit dose” is a discrete amount of the pharmaceutical composition comprising a predetermined amount of the active ingredient. The amount of the active ingredient is generally equal to the dosage of the active ingredient that would be administered to a subject or a convenient fraction of such a dosage such as, for example, one-half or one-third of such a dosage. The unit dosage form may be for a single daily dose or one of multiple daily doses (e.g., about 1 to 4 or more times per day). When multiple daily doses are used, the unit dosage form may be the same or different for each dose.

Although the descriptions of pharmaceutical compositions provided herein are principally directed to pharmaceutical compositions suitable for ethical administration to humans, it will be understood by the skilled artisan that such compositions are generally suitable for administration to animals of all sorts. Modification of pharmaceutical compositions suitable for administration to humans in order to render the compositions suitable for administration to various animals is well understood, and the ordinarily skilled veterinary pharmacologist can design and perform such modification with merely ordinary, if any, experimentation. Subjects to which administration of the pharmaceutical compositions of the invention is contemplated include, but are not limited to, humans and other primates, mammals including commercially relevant mammals such as cattle, pigs, horses, sheep, cats, and dogs.

In certain embodiments, the compositions of the invention are formulated using one or more pharmaceutically acceptable excipients or carriers. In certain embodiments, the pharmaceutical compositions of the invention comprise a therapeutically effective amount of at least one compound of the invention and a pharmaceutically acceptable carrier.

Pharmaceutically acceptable carriers, which are useful, include, but are not limited to, glycerol, water, saline, ethanol, recombinant human albumin (e.g., RECOMBUMIN®), solubilized gelatins (e.g., GELOFUSINE®), and other pharmaceutically acceptable salt solutions such as phosphates and salts of organic acids. Examples of these and other pharmaceutically acceptable carriers are described in Remington's Pharmaceutical Sciences (1991, Mack Publication Co., New Jersey).

The carrier may be a solvent or dispersion medium containing, for example, water, ethanol, polyol (for example, glycerol, propylene glycol, and liquid polyethylene glycol, and the like), recombinant human albumin, solubilized gelatins, suitable mixtures thereof, and vegetable oils. The proper fluidity may be maintained, for example, by the use of a coating such as lecithin, by the maintenance of the required particle size in the case of dispersion and by the use of surfactants. Prevention of the action of microorganisms may be achieved by various antibacterial and antifungal agents, for example, parabens, chlorobutanol, phenol, ascorbic acid, thimerosal, and the like. In many cases, isotonic agents, for example, sugars, sodium chloride, or polyalcohols such as mannitol and sorbitol, are included in the composition. Prolonged absorption of the injectable compositions may be brought about by including in the composition an agent that delays absorption, for example, aluminum monostearate or gelatin.

Formulations may be employed in admixtures with conventional excipients, i.e., pharmaceutically acceptable organic or inorganic carrier substances suitable for oral, parenteral, nasal, inhalational, intravenous, subcutaneous, transdermal enteral, or any other suitable mode of administration, known to the art. The pharmaceutical preparations may be sterilized and if desired mixed with auxiliary agents, e.g., lubricants, preservatives, stabilizers, wetting agents, emulsifiers, salts for influencing osmotic pressure buffers, coloring, flavoring and/or fragrance-conferring substances and the like. They may also be combined where desired with other active agents, e.g., other analgesic, anxiolytics or hypnotic agents. As used herein, “additional ingredients” include, but are not limited to, one or more ingredients that may be used as a pharmaceutical carrier.

The composition of the invention may comprise a preservative from about 0.005% to 2.0% by total weight of the composition. The preservative is used to prevent spoilage in the case of exposure to contaminants in the environment. Examples of preservatives useful in accordance with the invention include but are not limited to those selected from the group consisting of benzyl alcohol, sorbic acid, parabens, imidurea and combinations thereof. One such preservative is a combination of about 0.5% to 2.0% benzyl alcohol and 0.05% to 0.5% sorbic acid.

The composition may include an antioxidant and a chelating agent which inhibit the degradation of the compound. Antioxidants for some compounds are BHT, BHA, alpha-tocopherol and ascorbic acid in the exemplary range of about 0.01% to 0.3%, or BHT in the range of 0.03% to 0.1% by weight by total weight of the composition. The chelating agent may be present in an amount of from 0.01% to 0.5% by weight by total weight of the composition. Exemplary chelating agents include edetate salts (e.g. disodium edetate) and citric acid in the weight range of about 0.01% to 0.20%, or in the range of 0.02% to 0.10% by weight by total weight of the composition. The chelating agent is useful for chelating metal ions in the composition that may be detrimental to the shelf life of the formulation. While BHT and disodium edetate are exemplary antioxidant and chelating agent, respectively, for some compounds, other suitable and equivalent antioxidants and chelating agents may be substituted therefore as would be known to those skilled in the art.

Liquid suspensions may be prepared using conventional methods to achieve suspension of the active ingredient in an aqueous or oily vehicle. Aqueous vehicles include, for example, water, and isotonic saline. Oily vehicles include, for example, almond oil, oily esters, ethyl alcohol, vegetable oils such as arachis, olive, sesame, or coconut oil, fractionated vegetable oils, and mineral oils such as liquid paraffin. Liquid suspensions may further comprise one or more additional ingredients including, but not limited to, suspending agents, dispersing or wetting agents, emulsifying agents, demulcents, preservatives, buffers, salts, flavorings, coloring agents, and sweetening agents. Oily suspensions may further comprise a thickening agent. Known suspending agents include, but are not limited to, sorbitol syrup, hydrogenated edible fats, sodium alginate, polyvinylpyrrolidone, gum tragacanth, gum acacia, and cellulose derivatives such as sodium carboxymethylcellulose, methylcellulose, hydroxypropylmethyl cellulose. Known dispersing or wetting agents include, but are not limited to, naturally-occurring phosphatides such as lecithin, condensation products of an alkylene oxide with a fatty acid, with a long chain aliphatic alcohol, with a partial ester derived from a fatty acid and a hexitol, or with a partial ester derived from a fatty acid and a hexitol anhydride (e.g., polyoxyethylene stearate, heptadecaethyleneoxycetanol, polyoxyethylene sorbitol monooleate, and polyoxyethylene sorbitan monooleate, respectively). Known emulsifying agents include, but are not limited to, lecithin, acacia, and ionic or non ionic surfactants. Known preservatives include, but are not limited to, methyl, ethyl, or n-propyl para-hydroxybenzoates, ascorbic acid, and sorbic acid. Known sweetening agents include, for example, glycerol, propylene glycol, sorbitol, sucrose, and saccharin.

Liquid solutions of the active ingredient in aqueous or oily solvents may be prepared in substantially the same manner as liquid suspensions, the primary difference being that the active ingredient is dissolved, rather than suspended in the solvent. As used herein, an “oily” liquid is one which comprises a carbon-containing liquid molecule and which exhibits a less polar character than water. Liquid solutions of the pharmaceutical composition of the invention may comprise each of the components described with regard to liquid suspensions, it being understood that suspending agents will not necessarily aid dissolution of the active ingredient in the solvent. Aqueous solvents include, for example, water, and isotonic saline.

Oily solvents include, for example, almond oil, oily esters, ethyl alcohol, vegetable oils such as arachis, olive, sesame, or coconut oil, fractionated vegetable oils, and mineral oils such as liquid paraffin.

Powdered and granular formulations of a pharmaceutical preparation of the invention may be prepared using known methods. Such formulations may be administered directly to a subject, used, for example, to form tablets, to fill capsules, or to prepare an aqueous or oily suspension or solution by addition of an aqueous or oily vehicle thereto. Each of these formulations may further comprise one or more of dispersing or wetting agent, a suspending agent, ionic and non-ionic surfactants, and a preservative. Additional excipients, such as fillers and sweetening, flavoring, or coloring agents, may also be included in these formulations.

A pharmaceutical composition of the invention may also be prepared, packaged, or sold in the form of oil-in-water emulsion or a water-in-oil emulsion. The oily phase may be a vegetable oil such as olive or arachis oil, a mineral oil such as liquid paraffin, or a combination of these. Such compositions may further comprise one or more emulsifying agents such as naturally occurring gums such as gum acacia or gum tragacanth, naturally-occurring phosphatides such as soybean or lecithin phosphatide, esters or partial esters derived from combinations of fatty acids and hexitol anhydrides such as sorbitan monooleate, and condensation products of such partial esters with ethylene oxide such as polyoxyethylene sorbitan monooleate. These emulsions may also contain additional ingredients including, for example, sweetening or flavoring agents.

Methods for impregnating or coating a material with a chemical composition are known in the art, and include, but are not limited to methods of depositing or binding a chemical composition onto a surface, methods of incorporating a chemical composition into the structure of a material during the synthesis of the material (i.e., such as with a physiologically degradable material), and methods of absorbing an aqueous or oily solution or suspension into an absorbent material, with or without subsequent drying. Methods for mixing components include physical milling, the use of pellets in solid and suspension formulations and mixing in a transdermal patch, as known to those skilled in the art.

### Administration/Dosing

The regimen of administration may affect what constitutes an effective amount. The therapeutic formulations may be administered to the patient either prior to or after the onset of a disease or disorder. Further, several divided dosages, as well as staggered dosages may be administered daily or sequentially, or the dose may be continuously infused, or may be a bolus injection. Further, the dosages of the therapeutic formulations may be proportionally increased or decreased as indicated by the exigencies of the therapeutic or prophylactic situation.

Administration of the compositions of the present invention to a patient, such as a mammal, such as a human, may be carried out using known procedures, at dosages and for periods of time effective to treat a disease or disorder contemplated herein. An effective amount of the therapeutic compound necessary to achieve a therapeutic effect may vary according to factors such as the activity of the particular compound employed; the time of administration; the rate of excretion of the compound; the duration of the treatment; other drugs, compounds or materials used in combination with the compound; the state of the disease or disorder, age, sex, weight, condition, general health and prior medical history of the patient being treated, and like factors well-known in the medical arts. Dosage regimens may be adjusted to provide the optimum therapeutic response. For example, several divided doses may be administered daily or the dose may be proportionally reduced as indicated by the exigencies of the therapeutic situation. A non-limiting example of an effective dose range for a therapeutic compound of the invention is from about 0.01 mg/kg to 100 mg/kg of body weight/per day. One of ordinary skill in the art would be able to study the relevant factors and make the determination regarding the effective amount of the therapeutic compound without undue experimentation.

The compound may be administered to an animal as frequently as several times daily, or it may be administered less frequently, such as once a day, once a week, once every two weeks, once a month, or even less frequently, such as once every several months or even once a year or less. It is understood that the amount of compound dosed per day may be administered, in non-limiting examples, every day, every other day, every 2 days, every 3 days, every 4 days, or every 5 days. For example, with every other day administration, a 5 mg per day dose may be initiated on Monday with a first subsequent 5 mg per day dose administered on Wednesday, a second subsequent 5 mg per day dose administered on Friday, and so on. The frequency of the dose is readily apparent to the skilled artisan and depends upon a number of factors, such as, but not limited to, type and severity of the disease being treated, and type and age of the animal.

Actual dosage levels of the active ingredients in the pharmaceutical compositions of this invention may be varied so as to obtain an amount of the active ingredient that is effective to achieve the desired therapeutic response for a particular patient, composition, and mode of administration, without being toxic to the patient.

A medical doctor, e.g., physician or veterinarian, having ordinary skill in the art may readily determine and prescribe the effective amount of the pharmaceutical composition required. For example, the physician or veterinarian could start doses of the compounds of the invention employed in the pharmaceutical composition at levels lower than that required in order to achieve the desired therapeutic effect and gradually increase the dosage until the desired effect is achieved.

In particular embodiments, it is especially advantageous to formulate the compound in dosage unit form for ease of administration and uniformity of dosage. Dosage unit form as used herein refers to physically discrete units suited as unitary dosages for the patients to be treated; each unit containing a predetermined quantity of therapeutic compound calculated to produce the desired therapeutic effect in association with the required pharmaceutical vehicle. The dosage unit forms of the invention are dictated by and directly dependent on (a) the unique characteristics of the therapeutic compound and the particular therapeutic effect to be achieved, and (b) the limitations inherent in the art of compounding/formulating such a therapeutic compound for the treatment of a disease or disorder in a patient.

In certain embodiments, the compositions of the invention are administered to the patient in dosages that range from one to five times per day or more. In other embodiments, the compositions of the invention are administered to the patient in range of dosages that include, but are not limited to, once every day, every two days, every three days to once a week, and once every two weeks. It will be readily apparent to one skilled in the art that the frequency of administration of the various combination compositions of the invention will vary from subject to subject depending on many factors including, but not limited to, age, disease or disorder to be treated, gender, overall health, and other factors. Thus, the invention should not be construed to be limited to any particular dosage regime and the precise dosage and composition to be administered to any patient will be determined by the attending physician taking all other factors about the patient into account.

Compounds of the invention for administration may be in the range of from about 1 μg to about 7,500 mg, about 20 μg to about 7,000 mg, about 40 μg to about 6,500 mg, about 80 μg to about 6,000 mg, about 100 μg to about 5,500 mg, about 200 μg to about 5,000 mg, about 400 μg to about 4,000 mg, about 800 μg to about 3,000 mg, about 1 mg to about 2,500 mg, about 2 mg to about 2,000 mg, about 5 mg to about 1,000 mg, about 10 mg to about 750 mg, about 20 mg to about 600 mg, about 30 mg to about 500 mg, about 40 mg to about 400 mg, about 50 mg to about 300 mg, about 60 mg to about 250 mg, about 70 mg to about 200 mg, about 80 mg to about 150 mg, and any and all whole or partial increments there-in-between.

In some embodiments, the dose of a compound of the invention is from about 0.5 μg and about 5,000 mg. In some embodiments, a dose of a compound of the invention used in compositions described herein is less than about 5,000 mg, or less than about 4,000 mg, or less than about 3,000 mg, or less than about 2,000 mg, or less than about 1,000 mg, or less than about 800 mg, or less than about 600 mg, or less than about 500 mg, or less than about 200 mg, or less than about 50 mg. Similarly, in some embodiments, a dose of a second compound as described herein is less than about 1,000 mg, or less than about 800 mg, or less than about 600 mg, or less than about 500 mg, or less than about 400 mg, or less than about 300 mg, or less than about 200 mg, or less than about 100 mg, or less than about 50 mg, or less than about 40 mg, or less than about 30 mg, or less than about 25 mg, or less than about 20 mg, or less than about 15 mg, or less than about 10 mg, or less than about 5 mg, or less than about 2 mg, or less than about 1 mg, or less than about 0.5 mg, and any and all whole or partial increments thereof.

In certain embodiments, the present invention is directed to a packaged pharmaceutical composition comprising a container holding a therapeutically effective amount of a compound of the invention, alone or in combination with a second pharmaceutical agent; and instructions for using the compound to treat, prevent, or reduce one or more symptoms of a disease or disorder in a patient.

The term “container” includes any receptacle for holding the pharmaceutical composition or for managing stability or water uptake. For example, in certain embodiments, the container is the packaging that contains the pharmaceutical composition, such as liquid (solution and suspension), semisolid, lyophilized solid, solution and powder or lyophilized formulation present in dual chambers. In other embodiments, the container is not the packaging that contains the pharmaceutical composition, i.e., the container is a receptacle, such as a box or vial that contains the packaged pharmaceutical composition or unpackaged pharmaceutical composition and the instructions for use of the pharmaceutical composition.

Moreover, packaging techniques are well known in the art. It should be understood that the instructions for use of the pharmaceutical composition may be contained on the packaging containing the pharmaceutical composition, and as such the instructions form an increased functional relationship to the packaged product. However, it should be understood that the instructions may contain information pertaining to the compound's ability to perform its intended function, e.g., treating, preventing, or reducing a disease or disorder in a patient.

### Administration

Routes of administration of any of the compositions of the invention include inhalational, oral, nasal, rectal, parenteral, sublingual, transdermal, transmucosal (e.g., sublingual, lingual, (trans)buccal, (trans)urethral, vaginal (e.g., trans- and perivaginally), (intra)nasal, and (trans)rectal), intravesical, intrapulmonary, intraduodenal, intragastrical, intrathecal, epidural, intrapleural, intraperitoneal, subcutaneous, intramuscular, intradermal, intra-arterial, intravenous, intrabronchial, inhalation, and topical administration.

Suitable compositions and dosage forms include, for example, tablets, capsules, caplets, pills, gel caps, troches, emulsions, dispersions, suspensions, solutions, syrups, granules, beads, transdermal patches, gels, powders, pellets, magmas, lozenges, creams, pastes, plasters, lotions, discs, suppositories, liquid sprays for nasal or oral administration, dry powder or aerosolized formulations for inhalation, compositions and formulations for intravesical administration and the like. It should be understood that the formulations and compositions that would be useful in the present invention are not limited to the particular formulations and compositions that are described herein.

### Oral Administration

For oral application, particularly suitable are tablets, dragees, liquids, drops, capsules, caplets and gelcaps. Other formulations suitable for oral administration include, but are not limited to, a powdered or granular formulation, an aqueous or oily suspension, an aqueous or oily solution, a paste, a gel, toothpaste, a mouthwash, a coating, an oral rinse, or an emulsion. The compositions intended for oral use may be prepared according to any method known in the art and such compositions may contain one or more agents selected from the group consisting of inert, non-toxic, generally recognized as safe (GRAS) pharmaceutically excipients which are suitable for the manufacture of tablets. Such excipients include, for example an inert diluent such as lactose; granulating and disintegrating agents such as cornstarch; binding agents such as starch; and lubricating agents such as magnesium stearate.

Tablets may be non-coated or they may be coated using known methods to achieve delayed disintegration in the gastrointestinal tract of a subject, thereby providing sustained release and absorption of the active ingredient. By way of example, a material such as glyceryl monostearate or glyceryl distearate may be used to coat tablets. Further by way of example, tablets may be coated using methods described in U.S. Pat. Nos. 4,256,108; 4,160,452; and U.S. Pat. No. 4,265,874 to form osmotically controlled release tablets. Tablets may further comprise a sweetening agent, a flavoring agent, a coloring agent, a preservative, or some combination of these in order to provide for pharmaceutically elegant and palatable preparation. Hard capsules comprising the active ingredient may be made using a physiologically degradable composition, such as gelatin. The capsules comprise the active ingredient, and may further comprise additional ingredients including, for example, an inert solid diluent such as calcium carbonate, calcium phosphate, or kaolin.

Hard capsules comprising the active ingredient may be made using a physiologically degradable composition, such as gelatin. Such hard capsules comprise the active ingredient, and may further comprise additional ingredients including, for example, an inert solid diluent such as calcium carbonate, calcium phosphate, or kaolin.

Soft gelatin capsules comprising the active ingredient may be made using a physiologically degradable composition, such as gelatin from animal-derived collagen or from a hypromellose, a modified form of cellulose, and manufactured using optional mixtures of gelatin, water and plasticizers such as sorbitol or glycerol. Such soft capsules comprise the active ingredient, which may be mixed with water or an oil medium such as peanut oil, liquid paraffin, or olive oil.

For oral administration, the compounds of the invention may be in the form of tablets or capsules prepared by conventional means with pharmaceutically acceptable excipients such as binding agents; fillers; lubricants; disintegrates; or wetting agents. If desired, the tablets may be coated using suitable methods and coating materials such as OPADRY® film coating systems available from Colorcon, West Point, Pa. (e.g., OPADRY® OY Type, OYC Type, Organic Enteric OY—P Type, Aqueous Enteric OY-A Type, OY-PM Type and OPADRY® White, 32K18400). It is understood that similar type of film coating or polymeric products from other companies may be used.

A tablet comprising the active ingredient may, for example, be made by compressing or molding the active ingredient, optionally with one or more additional ingredients. Compressed tablets may be prepared by compressing, in a suitable device, the active ingredient in a free-flowing form such as a powder or granular preparation, optionally mixed with one or more of a binder, a lubricant, an excipient, a surface active agent, and a dispersing agent. Molded tablets may be made by molding, in a suitable device, a mixture of the active ingredient, a pharmaceutically acceptable carrier, and at least sufficient liquid to moisten the mixture. Pharmaceutically acceptable excipients used in the manufacture of tablets include, but are not limited to, inert diluents, granulating and disintegrating agents, binding agents, and lubricating agents. Known dispersing agents include, but are not limited to, potato starch and sodium starch glycolate. Known surface-active agents include, but are not limited to, sodium lauryl sulphate. Known diluents include, but are not limited to, calcium carbonate, sodium carbonate, lactose, microcrystalline cellulose, calcium phosphate, calcium hydrogen phosphate, and sodium phosphate. Known granulating and disintegrating agents include, but are not limited to, corn starch and alginic acid. Known binding agents include, but are not limited to, gelatin, acacia, pre-gelatinized maize starch, polyvinylpyrrolidone, and hydroxypropyl methylcellulose. Known lubricating agents include, but are not limited to, magnesium stearate, stearic acid, silica, and talc.

Granulating techniques are well known in the pharmaceutical art for modifying starting powders or other particulate materials of an active ingredient. The powders are typically mixed with a binder material into larger permanent free-flowing agglomerates or granules referred to as a “granulation.” For example, solvent-using “wet” granulation processes are generally characterized in that the powders are combined with a binder material and moistened with water or an organic solvent under conditions resulting in the formation of a wet granulated mass from which the solvent must then be evaporated.

Melt granulation generally consists in the use of materials that are solid or semi-solid at room temperature (i.e., having a relatively low softening or melting point range) to promote granulation of powdered or other materials, essentially in the absence of added water or other liquid solvents. The low melting solids, when heated to a temperature in the melting point range, liquefy to act as a binder or granulating medium. The liquefied solid spreads itself over the surface of powdered materials with which it is contacted, and on cooling, forms a solid granulated mass in which the initial materials are bound together. The resulting melt granulation may then be provided to a tablet press or be encapsulated for preparing the oral dosage form. Melt granulation improves the dissolution rate and bioavailability of an active (i.e., drug) by forming a solid dispersion or solid solution.

U.S. Pat. No. 5,169,645 discloses directly compressible wax-containing granules having improved flow properties. The granules are obtained when waxes are admixed in the melt with certain flow improving additives, followed by cooling and granulation of the admixture. In certain embodiments, only the wax itself melts in the melt combination of the wax(es) and additives(s), and in other cases both the wax(es) and the additives(s) will melt.

The present invention also includes a multi-layer tablet comprising a layer providing for the delayed release of one or more compounds useful within the methods of the invention, and a further layer providing for the immediate release of one or more compounds useful within the methods of the invention. Using a wax/pH-sensitive polymer mix, a gastric insoluble composition may be obtained in which the active ingredient is entrapped, ensuring its delayed release.

Liquid preparation for oral administration may be in the form of solutions, syrups or suspensions. The liquid preparations may be prepared by conventional means with pharmaceutically acceptable additives such as suspending agents (e.g., sorbitol syrup, methyl cellulose or hydrogenated edible fats); emulsifying agent (e.g., lecithin or acacia); non-aqueous vehicles (e.g., almond oil, oily esters or ethyl alcohol); and preservatives (e.g., methyl or propyl para-hydroxy benzoates or sorbic acid). Liquid formulations of a pharmaceutical composition of the invention which are suitable for oral administration may be prepared, packaged, and sold either in liquid form or in the form of a dry product intended for reconstitution with water or another suitable vehicle prior to use.

### Parenteral Administration

As used herein, “parenteral administration” of a pharmaceutical composition includes any route of administration characterized by physical breaching of a tissue of a subject and administration of the pharmaceutical composition through the breach in the tissue. Parenteral administration thus includes, but is not limited to, administration of a pharmaceutical composition by injection of the composition, by application of the composition through a surgical incision, by application of the composition through a tissue-penetrating non-surgical wound, and the like. In particular, parenteral administration is contemplated to include, but is not limited to, subcutaneous, intravenous, intraperitoneal, intramuscular, intrasternal injection, and kidney dialytic infusion techniques.

Formulations of a pharmaceutical composition suitable for parenteral administration comprise the active ingredient combined with a pharmaceutically acceptable carrier, such as sterile water or sterile isotonic saline. Such formulations may be prepared, packaged, or sold in a form suitable for bolus administration or for continuous administration. Injectable formulations may be prepared, packaged, or sold in unit dosage form, such as in ampules or in multidose containers containing a preservative. Injectable formulations may also be prepared, packaged, or sold in devices such as patient-controlled analgesia (PCA) devices. Formulations for parenteral administration include, but are not limited to, suspensions, solutions, emulsions in oily or aqueous vehicles, pastes, and implantable sustained-release or biodegradable formulations. Such formulations may further comprise one or more additional ingredients including, but not limited to, suspending, stabilizing, or dispersing agents. In one embodiment of a formulation for parenteral administration, the active ingredient is provided in dry (i.e., powder or granular) form for reconstitution with a suitable vehicle (e.g., sterile pyrogen-free water) prior to parenteral administration of the reconstituted composition.

The pharmaceutical compositions may be prepared, packaged, or sold in the form of a sterile injectable aqueous or oily suspension or solution. This suspension or solution may be formulated according to the known art, and may comprise, in addition to the active ingredient, additional ingredients such as the dispersing agents, wetting agents, or suspending agents described herein. Such sterile injectable formulations may be prepared using a non-toxic parenterally acceptable diluent or solvent, such as water or 1,3-butanediol, for example. Other acceptable diluents and solvents include, but are not limited to, Ringer's solution, isotonic sodium chloride solution, and fixed oils such as synthetic mono- or di-glycerides. Other parentally-administrable formulations which are useful include those which comprise the active ingredient in microcrystalline form in a recombinant human albumin, a fluidized gelatin, in a liposomal preparation, or as a component of a biodegradable polymer system. Compositions for sustained release or implantation may comprise pharmaceutically acceptable polymeric or hydrophobic materials such as an emulsion, an ion exchange resin, a sparingly soluble polymer, or a sparingly soluble salt.

### Topical Administration

An obstacle for topical administration of pharmaceuticals is the stratum corneum layer of the epidermis. The stratum corneum is a highly resistant layer comprised of protein, cholesterol, sphingolipids, free fatty acids and various other lipids, and includes cornified and living cells. One of the factors that limit the penetration rate (flux) of a compound through the stratum corneum is the amount of the active substance that can be loaded or applied onto the skin surface. The greater the amount of active substance which is applied per unit of area of the skin, the greater the concentration gradient between the skin surface and the lower layers of the skin, and in turn the greater the diffusion force of the active substance through the skin. Therefore, a formulation containing a greater concentration of the active substance is more likely to result in penetration of the active substance through the skin, and more of it, and at a more consistent rate, than a formulation having a lesser concentration, all other things being equal.

Formulations suitable for topical administration include, but are not limited to, liquid or semi-liquid preparations such as liniments, lotions, oil-in-water or water-in-oil emulsions such as creams, ointments or pastes, and solutions or suspensions. Topically administrable formulations may, for example, comprise from about 1% to about 10% (w/w) active ingredient, although the concentration of the active ingredient may be as high as the solubility limit of the active ingredient in the solvent. Formulations for topical administration may further comprise one or more of the additional ingredients described herein.

Enhancers of permeation may be used. These materials increase the rate of penetration of drugs across the skin. Typical enhancers in the art include ethanol, glycerol monolaurate, PGML (polyethylene glycol monolaurate), dimethylsulfoxide, and the like. Other enhancers include oleic acid, oleyl alcohol, ethoxydiglycol, laurocapram, alkanecarboxylic acids, dimethylsulfoxide, polar lipids, or N-methyl-2-pyrrolidone.

One acceptable vehicle for topical delivery of some of the compositions of the invention may contain liposomes. The composition of the liposomes and their use are known in the art (i.e., U.S. Pat. No. 6,323,219).

In alternative embodiments, the topically active pharmaceutical composition may be optionally combined with other ingredients such as adjuvants, anti-oxidants, chelating agents, surfactants, foaming agents, wetting agents, emulsifying agents, viscosifiers, buffering agents, preservatives, and the like. In other embodiments, a permeation or penetration enhancer is included in the composition and is effective in improving the percutaneous penetration of the active ingredient into and through the stratum corneum with respect to a composition lacking the permeation enhancer. Various permeation enhancers, including oleic acid, oleyl alcohol, ethoxydiglycol, laurocapram, alkanecarboxylic acids, dimethylsulfoxide, polar lipids, or N-methyl-2-pyrrolidone, are known to those of skill in the art. In another aspect, the composition may further comprise a hydrotropic agent, which functions to increase disorder in the structure of the stratum corneum, and thus allows increased transport across the stratum corneum. Various hydrotropic agents such as isopropyl alcohol, propylene glycol, or sodium xylene sulfonate, are known to those of skill in the art.

The topically active pharmaceutical composition should be applied in an amount effective to affect desired changes. As used herein “amount effective” shall mean an amount sufficient to cover the region of skin surface where a change is desired. An active compound should be present in the amount of from about 0.0001% to about 15% by weight volume of the composition. For example, it should be present in an amount from about 0.0005% to about 5% of the composition; for example, it should be present in an amount of from about 0.001% to about 1% of the composition. Such compounds may be synthetically—or naturally derived.

### Buccal Administration

A pharmaceutical composition of the invention may be prepared, packaged, or sold in a formulation suitable for buccal administration. Such formulations may, for example, be in the form of tablets or lozenges made using conventional methods, and may contain, for example, 0.1 to 20% (w/w) of the active ingredient, the balance comprising an orally dissolvable or degradable composition and, optionally, one or more of the additional ingredients described herein. Alternately, formulations suitable for buccal administration may comprise a powder or an aerosolized or atomized solution or suspension comprising the active ingredient. Such powdered, aerosolized, or aerosolized formulations, when dispersed, may have an average particle or droplet size in the range from about 0.1 to about 200 nanometers, and may further comprise one or more of the additional ingredients described herein. The examples of formulations described herein are not exhaustive and it is understood that the invention includes additional modifications of these and other formulations not described herein, but which are known to those of skill in the art.

### Rectal Administration

A pharmaceutical composition of the invention may be prepared, packaged, or sold in a formulation suitable for rectal administration. Such a composition may be in the form of, for example, a suppository, a retention enema preparation, and a solution for rectal or colonic irrigation.

Suppository formulations may be made by combining the active ingredient with a non-irritating pharmaceutically acceptable excipient which is solid at ordinary room temperature (i.e., about 20° C.) and which is liquid at the rectal temperature of the subject (i.e., about 37° C. in a healthy human). Suitable pharmaceutically acceptable excipients include, but are not limited to, cocoa butter, polyethylene glycols, and various glycerides. Suppository formulations may further comprise various additional ingredients including, but not limited to, antioxidants, and preservatives.

Retention enema preparations or solutions for rectal or colonic irrigation may be made by combining the active ingredient with a pharmaceutically acceptable liquid carrier. As is well known in the art, enema preparations may be administered using, and may be packaged within, a delivery device adapted to the rectal anatomy of the subject. Enema preparations may further comprise various additional ingredients including, but not limited to, antioxidants, and preservatives.

### Additional Administration Forms

Additional dosage forms of this invention include dosage forms as described in U.S. Pat. Nos. 6,340,475, 6,488,962, 6,451,808, 5,972,389, 5,582,837, and 5,007,790. Additional dosage forms of this invention also include dosage forms as described in U.S. Patent Applications Nos. 20030147952, 20030104062, 20030104053, 20030044466, 20030039688, and 20020051820. Additional dosage forms of this invention also include dosage forms as described in PCT Applications Nos. WO 03/35041, WO 03/35040, WO 03/35029, WO 03/35177, WO 03/35039, WO 02/96404, WO 02/32416, WO 01/97783, WO 01/56544, WO 01/32217, WO 98/55107, WO 98/11879, WO 97/47285, WO 93/18755, and WO 90/11757.

### Controlled Release Formulations and Drug Delivery Systems:

In certain embodiments, the compositions and/or formulations of the present invention may be, but are not limited to, short-term, rapid-offset, as well as controlled, for example, sustained release, delayed release and pulsatile release formulations.

The term sustained release is used in its conventional sense to refer to a drug formulation that provides for gradual release of a drug over an extended period of time, and that may, although not necessarily, result in substantially constant blood levels of a drug over an extended time period. The period of time may be as long as a month or more and should be a release which is longer that the same amount of agent administered in bolus form.

For sustained release, the compounds may be formulated with a suitable polymer or hydrophobic material which provides sustained release properties to the compounds. As such, the compounds for use the method of the invention may be administered in the form of microparticles, for example, by injection or in the form of wafers or discs by implantation.

In certain embodiments of the invention, the compounds useful within the invention are administered to a subject, alone or in combination with another pharmaceutical agent, using a sustained release formulation.

The term delayed release is used herein in its conventional sense to refer to a drug formulation that provides for an initial release of the drug after some delay following drug administration and that may, although not necessarily, include a delay of from about 10 minutes up to about 12 hours.

The term pulsatile release is used herein in its conventional sense to refer to a drug formulation that provides release of the drug in such a way as to produce pulsed plasma profiles of the drug after drug administration.

The term immediate release is used in its conventional sense to refer to a drug formulation that provides for release of the drug immediately after drug administration.

As used herein, short-term refers to any period of time up to and including about 8 hours, about 7 hours, about 6 hours, about 5 hours, about 4 hours, about 3 hours, about 2 hours, about 1 hour, about 40 minutes, about 20 minutes, or about 10 minutes and any or all whole or partial increments thereof after drug administration after drug administration.

As used herein, rapid-offset refers to any period of time up to and including about 8 hours, about 7 hours, about 6 hours, about 5 hours, about 4 hours, about 3 hours, about 2 hours, about 1 hour, about 40 minutes, about 20 minutes, or about 10 minutes, and any and all whole or partial increments thereof after drug administration.

Those skilled in the art will recognize, or be able to ascertain using no more than routine experimentation, numerous equivalents to the specific procedures, embodiments, claims, and examples described herein. Such equivalents were considered to be within the scope of this invention and covered by the claims appended hereto. For example, it should be understood, that modifications in reaction conditions, including but not limited to reaction times, reaction size/volume, and experimental reagents, such as solvents, catalysts, pressures, atmospheric conditions, e.g., nitrogen atmosphere, and reducing/oxidizing agents, with art-recognized alternatives and using no more than routine experimentation, are within the scope of the present application.

It is to be understood that, wherever values and ranges are provided herein, the description in range format is merely for convenience and brevity and should not be construed as an inflexible limitation on the scope of the invention. Accordingly, all values and ranges encompassed by these values and ranges are meant to be encompassed within the scope of the present invention. Moreover, all values that fall within these ranges, as well as the upper or lower limits of a range of values, are also contemplated by the present application. The description of a range should be considered to have specifically disclosed all the possible sub-ranges as well as individual numerical values within that range and, when appropriate, partial integers of the numerical values within ranges. For example, description of a range such as from 1 to 6 should be considered to have specifically disclosed sub-ranges such as from 1 to 3, from 1 to 4, from 1 to 5, from 2 to 4, from 2 to 6, from 3 to 6 etc., as well as individual numbers within that range, for example, 1, 2, 2.7, 3, 4, 5, 5.3, and 6. This applies regardless of the breadth of the range.

The following examples further illustrate aspects of the present invention. However, they are in no way a limitation of the teachings or disclosure of the present invention as set forth herein.

### EXAMPLES

The invention is now described with reference to the following Examples. These Examples are provided for the purpose of illustration only, and the invention is not limited to these Examples, but rather encompasses all variations that are evident as a result of the teachings provided herein.

### LC-MS Methods

LCMS Method A: The column used for chromatography was a Luna-C18 2.0*30 mm, (3 um particle size). Detection methods are diode array (DAD). MS mode was positive electrospray ionization. MS range was 100-1000. Mobile phase A was 0.037% Trifluoroacetic acid in water, and mobile phase B was 0.018% Trifluoroacetic acid in HPLC grade acetonitrile. The gradient was 10-80% B in 2.00 min 0.10% B in 0.01 min, 10-80% B (0.01-1.15 min) with a hold at 80% B for 0.50 min, 80-10% B (1.65-1.66 min) with a hold at 10% B for 0.34 min. The flow rate was 0.8 mL/min (0.01-1.51 min) and 1.2 mL/min (1.52-2.00 min).

LCMS Method B: The column used for chromatography was Xtimate C18 2.1*30 mm, (3 um particle size). Detection methods are diode array (DAD). MS mode was positive electrospray ionization. MS range was 100-1000. Mobile phase A was 0.037% trifluoroacetic acid in water, and mobile phase B was 0.018% trifluoroacetic acid in HPLC grade acetonitrile. The gradient was 5-95% B in 2.00 min 0.5% B in 0.00 min, 5-95% B (0.00-0.80 min) 95-100% B (0.80-1.60 min) 100-5% B (1.60-1.61 min) with a hold at 5% B for 0.39 min. The flow rate was 1.2 mL/min.

LCMS Method C: The column used for chromatography was Xtimate C18 2.1*30 mm, (3 um particle size). Detection methods are diode array (DAD). MS mode was positive electrospray ionization. MS range was 100-1000. Mobile phase A was 0.037% Trifluoroacetic acid in water, and mobile phase B was 0.018% Trifluoroacetic acid in HPLC grade acetonitrile. The gradient was 5-95% B in 2.00 min 0.5% B in 0.00 min, 5-95% B (0.00-1.00 min) 95-100% B (1.00-1.80 min) 100-5% B (1.80-1.81 min) with a hold at 5% B for 0.19 min. The flow rate was 1.0 mL/min (0.00-1.80 min), 1.2 mL/min (1.81-2.00 min).

LCMS Method F: The column used for chromatography was Xtimate C18 2.1*30 mm, (3 um particle size). Detection methods are diode array (DAD). MS mode was positive electrospray ionization. MS range was 100-1000. Mobile phase A was 0.037% trifluoroacetic acid in water, and mobile phase B was 0.018% trifluoroacetic acid in HPLC grade acetonitrile. The gradient was 5-95% B in 2.00 min 0.5% B in 0.00 min, 5-95% B (0.00-0.80 min) 95-100% B (0.80-1.60 min) 100-5% B (1.60-1.61 min) with a hold at 5% B for 0.39 min. The flow rate was 1.2 mL/min.

LCMS Method J: LC/MS (The column used for chromatography was Xtimate C18 2.1*30 mm, (3 um particle size). Detection methods are diode array (DAD). MS mode was positive electrospray ionization. MS range was 100-1000. Mobile phase A was 0.037% Trifluoroacetic acid in water, and mobile phase B was 0.018% Trifluoroacetic acid in HPLC grade acetonitrile. The gradient was 5-95% B in 2.00 min 0.5% B in 0.00 min, 5-95% B (0.00-1.00 min) 95-100% B (1.00-1.80 min) 100-5% B (1.80-1.81 min) with a hold at 5% B for 0.19 min. The flow rate was 1.0 mL/min (0.00-1.80 min), 1.2 mL/min (1.81-2.00 min).

LCMS Method K: The column used for chromatography was Xbridge Shield RP18 2.1*50 mm, (5 um particle size). Detection methods are diode array (DAD). MS mode was positive electrospray ionization. MS range was 100-1000. Mobile phase A was 10 mM Ammonium bicarbonate in water, and mobile phase B was HPLC grade acetonitrile. The gradient was 10-90% B in 2.00 min 0.10% B in 0.01 min, 10-90% B (0.01-1.15 min) with a hold at 90% B for 0.50 min, 90-10% B (1.65-1.66 min) with a hold at 10% B for 0.54 min. The flow rate was 0.8 mL/min (0.01-1.51 min) and 1.2 mL/min (1.52-2.00 min).

LCMS Method Q: LC/MS (The column used for chromatography was a Luna-C18 2.0*30 mm, (3 um particle size). Detection methods are diode array (DAD) and evaporative light scattering (ELSD). MS mode was positive electrospray ionization. MS range was 100-1000. Mobile phase A was 0.037% Trifluoroacetic acid in water, and mobile phase B was 0.018% Trifluoroacetic acid i in HPLC grade acetonitrile. The gradient was 10-90% B in 2.00 min 0.10% B in 0.01 min, 10-90% B (0.01-1.15 min) with a hold at 90% B for 0.50 min, 90-10% B (1.65-1.66 min) with a hold at 10% B for 0.54 min. The flow rate was 0.8 mL/min (0.01-1.51 min) and 1.2 mL/min (1.52-2.00 min).

LCMS Method AB: LC/MS (The column used for chromatography was a Chromolith RP-18e 25-2 mm. Detection methods are diode array (DAD). MS mode was positive electrospray ionization. MS range was 100-1000. Mobile phase A was 0.037% Trifluoroacetic acid in water, and mobile phase B was 0.018% Trifluoroacetic acid in HPLC grade acetonitrile. The gradient was 5-95% B in 1.5 min 0.5% B in 0.01 min, 5-95% B (0.01-0.7 min), 95% B (0.7-1.15 min), 5% B in 1.16 min with a hold at 5% B for 0.34 min. The flow rate was 1.5 mL/min (0.00-1.5 min).

LCMS Method AD: The column used for chromatography was a Chromolith RP-18e 25-2 mm. Detection methods are diode array (DAD). MS mode was positive electrospray ionization. MS range was 100-1000. Mobile phase A was 0.037% trifluoroacetic acid in water, and mobile phase B was 0.018% Trifluoroacetic acid in HPLC grade acetonitrile. The gradient was 5-95% B in 1.5 min 0.5% B in 0.01 min, 5-95% B (0.01-0.7 min), 95% B (0.7-1.15 min), 5% B in 1.16 min with a hold at 5% B for 0.34 min. The flow rate was 1.5 mL/min (0.00-1.5 min).

LCMS Method AE: The column used for chromatography was a Luna-C18 2.0*30 mm, (3 um particle size). Detection methods are diode array (DAD). MS mode was positive electrospray ionization. MS range was 100-1000. Mobile phase A was 0.037% Trifluoroacetic acid in water, and mobile phase B was 0.018% Trifluoroacetic acid in HPLC grade acetonitrile. The gradient was 10-80% B in 2.00 min 0.10% B in 0.01 min, 10-80% B (0.01-1.15 min) with a hold at 80% B for 0.50 min, 80-10% B (1.65-1.66 min) with a hold at 10% B for 0.34 min. The flow rate was 0.8 mL/min (0.01-1.51 min) and 1.2 mL/min (1.52-2.00 min).

LCMS Method AF: The column used for chromatography was a Kinetex EVO C18 30*2.1 mm, (Sum particle size). Detection methods are diode array (DAD). MS mode was positive electrospray ionization. MS range was 100-1000. Mobile phase A was 0.037% TFA in water, and mobile phase B was 0.018% TFA in HPLC grade acetonitrile. The gradient was 5-95% B in 1.50 min 0.5% B in 0.01 min, 5-95% B (0.01-0.80 min) with a hold at 95% B for 0.40 min, 5% B at 1.21 min with a hold at 5% B for 0.29 min. The flow rate was 1.5 mL/min (0.00-1.50 min).

LCMS Method AG: The column used for chromatography was Xbridge Shield RP18 2.1*50 mm, (5 um particle size). Detection methods are diode array (DAD). MS mode was positive electrospray ionization. MS range was 100-1000. Mobile phase A was 10 mM Ammonium bicarbonate in water, and mobile phase B was HPLC grade acetonitrile. The gradient was 5-95% B in 4.30 min 0.5% B in 0.01 min, 5-95% B (0.01-3.00 min), and hold at 95% B within 0.5 min, 95-5% B (3.50-3.51 min), with a hold at 5% B for 0.79 min. The flow rate was1.OmL/min (0.01-4.30 min)

Method AH: LC/MS (The column used for chromatography was a Luna-C18 2.0*30 mm, (3 um particle size). Detection methods are diode array (DAD). MS mode was positive electrospray ionization. MS range was 100-1000. Mobile phase A was 0.037% trifluoroacetic acid in water, and mobile phase B was 0.018% trifluoroacetic acid in HPLC grade acetonitrile. The gradient was 10-80% B in 4.30 min 0.10% B in 0.01 min, 10-80% B (0.01-3.50 min), 80-10% B (3.50-3.80 min), with a hold at 10% B for 0.50 min. The flow rate was 0.8 mL/min (0.01-3.80 min) and 1.2 mL/min (3.81-4.30 min).

Method AL: LC/MS (The column used for chromatography was Xbridge Shield RP18 2.1*50 mm, (5 um particle size). Detection methods are diode array (DAD). MS mode was positive electrospray ionization. MS range was 100-1000. Mobile phase A was 10 mM Ammonium bicarbonate in water, and mobile phase B was HPLC grade acetonitrile. The gradient was 10-90% B in 4.30 min 0.10% B in 0.01 min, 10-90% B (0.01-3.50 min), 90-10% 5B (3.50-3.80 min), with a hold at 10% B for 0.50 min. The flow rate was 0.8 mL/min (0.01-3.80 min) and 1 mL/min (3.80-4.30 min).

### Example 1: 5-((4-Chloro-2-(((2-hydroxyethyl)amino)methyl)-5-((3′-(3-(((2-hydroxyethyl)amino)methyl)-4-oxo-4H-pyrido[1,2-a]pyrimidin-8-yl)-2-methyl-[1,1′-biphenyl]-3-yl)methoxy)phenoxy)methyl)nicotinamide

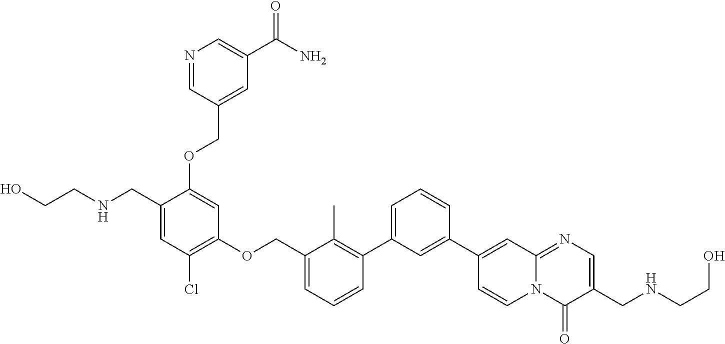 [SMILES: Cc1c(COc2cc(OCc3cncc(C(N)=O)c3)c(CNCCO)cc2Cl)cccc1-c1cccc(-c2ccn3c(=O)c(CNCCO)cnc3c2)c1]

### 5-Chloro-2,4-dihydroxy-benzaldehyde

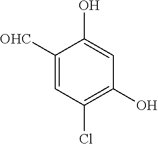 [SMILES: O=Cc1cc(Cl)c(O)cc1O]

To a mixture of 2,4-dihydroxybenzaldehyde (20 g, 144.80 mmol, 1 eq) in THE (300 mL) was added dropwise sulfuryl chloride (19.54 g, 144.80 mmol, 1 eq) at −30° C., and then the solution was stirred at 25° C. for 12 hr. The reaction was quenched with sat. NaHCO₃ to pH=7 and concentrated. The residue was purified by column chromatography (silica gel, petroleum ether:ethyl acetate=8:1 to 3:1) to give a semi-purified product (17 g). Portion of this product (10 g, 57.95 mmol, 1 eq) was treated with NaOH (1N, 200 mL) and the mixture was stirred at 25° C. for 1 hr. The mixture was extracted with EtOAc (2×200 mL). The aqueous solution was acidified with 3 N hydrogen chloride solution to pH=1. The solid was collected by filtration and washed with 100 mL of H₂O to give 5-chloro-2,4-dihydroxy-benzaldehyde (9 g, 90.0% yield) as a grey solid. ¹H NMR (400 MHz, CDCl₃): δ 11.25 (s, 1H), 9.69 (s, 1H), 7.52 (s, 1H), 6.61 (s, 1H).

### 1-Bromo-3-(bromomethyl)-2-methyl-benzene

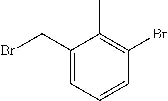

A solution of (3-bromo-2-methyl-phenyl)methanol (5 g, 24.87 mmol, 1 eq) in hydrobromic in AcOH (1 M, 50 mL) was degassed and purged with N₂ for 3 times. The mixture was stirred under N₂ at 90° C. for 1 hr. The reaction mixture was filtered and concentrated under reduced pressure to give 1-bromo-3-(bromomethyl)-2-methyl-benzene (6 g, 91.41% yield) as a yellow solid that was used directly for next step.

### 4-[(3-Bromo-2-methyl-phenyl)methoxy]-5-chloro-2-hydroxy-benzaldehyde

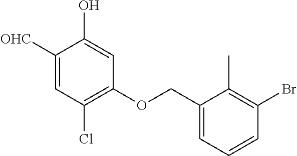 [SMILES: Cc1c(Br)cccc1COc1cc(O)c(C=O)cc1Cl]

To a mixture of 5-chloro-2,4-dihydroxy-benzaldehyde (9 g, 52.15 mmol, 1 eq) and K₂CO₃ (14.42 g, 104.31 mmol, 2 eq) in MeCN (80 mL) was added dropwise a solution of 1-bromo-3-(bromomethyl)-2-methyl-benzene (8.67 g, 32.85 mmol, 0.63 eq) in MeCN (20 mL). The reaction mixture was stirred at 25° C. for 12 hr. To the mixture was added H₂O until all the white K₂CO₃ was dissolved and the remaining yellow solid was collected by filtration. The yellow solid was treated with EtOAc (100 mL) and water (100 mL). The resulting mixture was stirred at 25° C. for 0.5 hr. Then the mixture was filtered and the filter cake was washed with EtOAc (20 mL). The solid was dried under vacuum to give the 4-[(3-bromo-2-methyl-phenyl)methoxy]-5-chloro-2-hydroxy-benzaldehyde (13 g) as a grey solid. ¹H NMR (400 MHz, CDCl₃) δ 11.43 (s, 1H), 9.72 (s, 1H), 7.62-7.56 (m, 1H), 7.44-7.42 (m, 2H), 7.28 (s, 1H), 7.14-7.12 (t, 1H, J=8 Hz), 6.60 (s, 1H), 5.18 (s, 2H), 2.47 (s, 3H).

### 5-(Hydroxymethyl)pyridine-3-carbonitrile

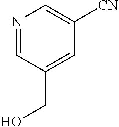

A solution of (5-bromo-3-pyridyl)methanol (3 g, 15.96 mmol, 1 eq), Zn(CN)₂ (2.06 g, 17.55 mmol, 1.1 eq), DPPF (884.55 mg, 1.60 mmol, 0.1 eq), Zn (104.33 mg, 1.60 mmol, 0.1 eq) and Pd(dppf)Cl₂ (1.17 g, 1.60 mmol, 0.1 eq) in DME (40 mL) was degassed and purged with N₂ for 3 times. The mixture was stirred at 130° C. for 15 hr in sealed tube under N₂. The reaction mixture was filtered and concentrated. The residue was purified by column chromatography (silica gel, petroleum ether:ethyl acetate=40:1 to 0:1) to give 5-(hydroxymethyl)pyridine-3-carbonitrile (1 g, 13.86% yield) as a black solid that was used for next step without further purification. MS: m/z found 135.1 [M+H]⁺; ¹H NMR (400 MHz, CDCl₃): δ 8.72 (s, 2H), 7.95 (s, 1H), 4.75 (s, 2H).

### 5-(Chloromethyl)pyridine-3-carbonitrile

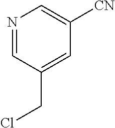

To a solution of 5-(hydroxymethyl)pyridine-3-carbonitrile (2 g, 14.91 mmol, 1 eq) in DCM (10 mL) was added hydrogen chloride in dioxane (4 M, 5 mL) and concentrated. To the residue was added SOCl₂ (10.66 g, 89.61 mmol, 6.50 mL, 6.01 eq) and the resulting solution was stirred for 3 hr at 60° C. After cooling, toluene (200 mL) was added and the mixture was filtered. The solid was treated directly with sat. NaHCO₃ to pH=7 and extracted with DCM (3×50 mL). The combined DCM solution was dried over Na₂SO₄ and concentrated under reduced pressure to give 5-(chloromethyl)pyridine-3-carbonitrile (1.7 g, 74.72% yield) as a black solid that was used for next step without further purification.

### 5-[[5-[(3-Bromo-2-methyl-phenyl)methoxy]-4-chloro-2-formyl-phenoxy]methyl]pyridine-3-carbonitrile

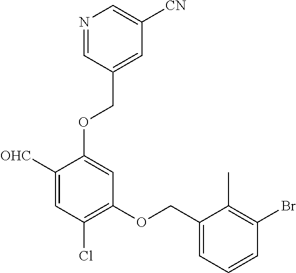 [SMILES: Cc1c(Br)cccc1COc1cc(OCc2cncc(C#N)c2)c(C=O)cc1Cl]

A solution of 4-[(3-bromo-2-methyl-phenyl)methoxy]-5-chloro-2-hydroxy-benzaldehyde (12.5 g, 35.15 mmol, 1 eq), 5-(chloromethyl)pyridine-3-carbonitrile (4.02 g, 26.36 mmol, 0.75 eq), Cs₂CO₃ (13.74 g, 42.18 mmol, 1.2 eq) and NaI (0.53 g, 3.52 mmol, 0.1 eq) in DMF (150 mL) was stirred for 3 hr at 75° C. The mixture was poured into ice-H₂O (500 mL) and stirred for 20 min. The precipitate was collected and washed with cold EtOAc (100 mL) and dried under vacuum. The solid was suspended in EtOAc (150 mL) and stirred for 1 hr at 25° C. Then the mixture was filtered and the filter cake was washed with 100 mL of EtOAc. The solid was dried to give 5-[[5-[(3-bromo-2-methyl-phenyl)methoxy]-4-chloro-2-formyl-phenoxy]methyl]-pyridine-3-carbonitrile (9.5 g) as a grey solid that was used for next step 5 without further purification. LC-MS: m/z found 472.9 [M+H]⁺.

### 5-[[4-Chloro-2-formyl-5-[[2-methyl-3-(4,4,5,5-tetramethyl-1,3,2-dioxaborolan-2-yl)phenyl]methoxy]phenoxy]methyl]pyridine-3-carbonitrile

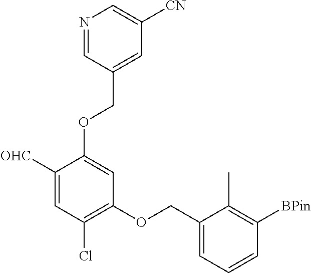

To a solution of 5-[[5-[(3-bromo-2-methyl-phenyl)methoxy]-4-chloro-2-formyl-phenoxy]methyl]pyridine-3-carbonitrile (5 g, 10.60 mmol, 1 eq) and 4,4,5,5-tetramethyl-2-(4,4,5,5-tetramethyl-1,3,2-dioxaborolan-2-yl)-1,3,2-dioxaborolane (5.38 g, 21.20 mmol, 2 eq) in dioxane (50 mL) was added KOAc (3.12 g, 31.80 mmol, 3 eq) and Pd(dppf)Cl₂ (0.77 g, 1.06 mmol, 0.1 eq). The mixture was stirred at 90° C. for 12 hr. The reaction mixture combined with another batch at same scale was filtered and concentrated to give the residue. The residue was purified by column chromatography (silica gel, Petroleum ether/Ethyl acetate=5/1 to 1:1). 8 g of 5-[[4-chloro-2-formyl-5-[[2-methyl-3-(4,4,5,5-tetramethyl-1,3,2-dioxaborolan-2-yl)phenyl]methoxy]phenoxy]methyl]pyridine-3-carbonitrile was obtained as a white solid. MS: m/z found 519.1 [M+H]⁺; ¹H NMR (400 MHz, DMSO-d₆): δ 10.21 (s, 1H), 9.02-9.01 (m, 2H), 8.54-8.52 (m, 1H), 7.71 (s, 1H), 7.64-7.62 (d, 1H, J=7.2 Hz), 7.56-7.54 (d, 1H, J=6.4 Hz), 7.22-7.15 (m, 2H), 5.47 (s, 2H), 5.34 (s, 2H), 2.51 (s, 3H), 1.30 (s, 12H).

### Diethyl 2-(((4-bromopyridin-2-yl)amino)methylene)malonate

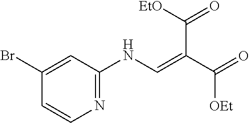 [SMILES: CCOC(=O)C(=CNc1cc(Br)ccn1)C(=O)OCC]

A mixture of 4-bromopyridin-2-amine (20 g, 116 mmol) and diethyl 2-(ethoxymethylene)malonate (23 mL, 116 mmol) was stirred at 120° C. for 30 hrs. The mixture was concentrated under reduced pressure. The residue was triturated with cyclohexane (50 mL) to give a solid. The resulting yellow solid was collected by filtration to afford diethyl 2-(((4-bromopyridin-2-yl)amino)methylene)malonate (25 g, 63% yield) as a yellow solid. ¹H NMR (400 MHz, DMSO-d₆): δ 10.70 (d, J=12.8 Hz, 1H), 8.97 (d, J=12.8 Hz, 1H), 8.23 (d, J=5.2 Hz, 1H), 7.77 (s, 1H), 7.37-7.36 (m, 1H), 4.24-4.11 (m, 4H), 1.27-1.16 (m, 6H).

### Ethyl 8-bromo-4-oxo-4H-pyrido[1,2-a]pyrimidine-3-carboxylate

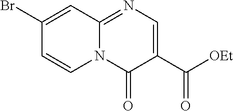 [SMILES: CCOC(=O)c1cnc2cc(Br)ccn2c1=O]

The solution of diethyl 2-(((4-bromopyridin-2-yl)amino)methylene)malonate (5 g, 14.6 mmol) in oxydibenzene (20 mL) was stirred at 250° C. for 2 hrs. The mixture was cooled to 40° C., triturated with cyclohexane (50 mL) at 40° C. for 30 min. The resulting yellow solid was collected by filtration to afford ethyl 8-bromo-4-oxo-4H-pyrido[1,2-a]pyrimidine-3-carboxylate (3.5 g, 81% yield) as a yellow solid. ¹H NMR (400 MHz, DMSO-d₆) δ 8.97 (d, J=7.6 Hz, 1H), 8.82 (s, 1H), 8.16 (d, J=1.6 Hz, 1H), 7.71-7.68 (m, 1H), 4.28-4.23 (m, 2H), 1.28 (t, J=7.20 Hz, 3H).

### Ethyl 8-(3-bromophenyl)-4-oxo-4H-pyrido[1,2-a]pyrimidine-3-carboxylate

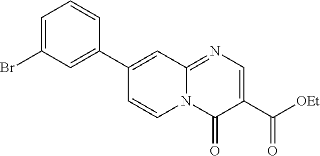 [SMILES: CCOC(=O)c1cnc2cc(-c3cccc(Br)c3)ccn2c1=O]

To a solution of ethyl 8-bromo-4-oxo-4H-pyrido[1,2-a]pyrimidine-3-carboxylate (3 g, 10 mmol) in toluene (30 mL), ethanol (8 mL) and water (7 mL) was added (3-bromophenyl)boronic acid (1.6 g, 8 mmol), sodium carbonate (2 g, 20 mmol) and tetrakis(triphenylphosphine)palladium (1.2 g, 1 mmol). The mixture was stirred at 80° C. for 4 hrs. The reaction mixture was concentrated under reduced pressure. To the residue was added water (20 mL) and the mixture was extracted with EtOAc (3×20 mL). The combined organic layers were washed with saturated aqueous brine solution (2×20 mL), dried over anhydrous sodium sulfate, filtered and concentrated under reduced pressure. The residue was purified by normal phase flash SiO₂ chromatography (0˜100% EtOAc/petroleum ether) to afford ethyl 8-(3-bromophenyl)-4-oxo-4H-pyrido[1,2-a]pyrimidine-3-carboxylate (1.8 g, 48% yield) as a yellow solid. ¹H NMR (400 MHz, DMSO-d₆) δ 9.14 (d, J=7.2 Hz, 1H), 8.88 (s, 1H), 8.23-8.19 (m, 2H), 8.05-7.92 (m, 2H), 7.79-7.74 (m, 1H), 7.55 (t, J=8.0 Hz, 1H), 4.26 (q, J=7.2 Hz, 2H), 1.29 (t, J=7.2 Hz, 3H).

### 8-(3-Bromophenyl)-3-(hydroxymethyl)-4H-pyrido[1,2-a]pyrimidin-4-one

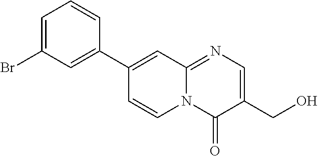 [SMILES: O=c1c(CO)cnc2cc(-c3cccc(Br)c3)ccn12]

To a solution of ethyl 8-(3-bromophenyl)-4-oxo-pyrido[1,2-a]pyrimidine-3-carboxylate (1 g, 2.7 mmol) in dichloromethane (40 mL) and ether (40 mL) was added diisobutylaluminum hydride (5.4 mL, 5.5 mmol, 1 M in toluene) dropwise at −10° C. (over 15 min). The mixture was stirred under nitrogen gas at −10° C. for 1 hr and at r.t. for 1 hr. The mixture was cooled to −10° C., more diisobutylaluminum hydride (1.0 mL, 1 mmol, 1 M in toluene) was added and the mixture was allowed to warm to r.t. and stirred for 5 hrs. The mixture was cooled to 0° C. and quenched carefully with 1 N aqueous HCl solution (6 mL). The mixture was diluted with dichloromethane (50 mL) and separated. The organic phase was washed with saturated aqueous brine solution (2×20 mL), dried over anhydrous sodium sulfate, filtered and concentrated under reduced pressure. The residue was purified by normal phase flash silica gel chromatography (THF/EtOAc/Petroleum ether=1/1/18-2/2/1) to afford 8-(3-bromophenyl)-3-(hydroxymethyl)-4H-pyrido[1,2-a]pyrimidin-4-one (260 mg, 29% yield) as a yellow solid. ¹H NMR (400 MHz, DMSO-d₆) δ 8.97 (d, J=7.6 Hz, 1H), 8.39 (s, 1H), 8.18 (s, 1H), 8.05 (s, 1H), 8.02-7.97 (m, 1H), 7.78-7.73 (m, 2H), 7.54 (t, J=8.0 Hz, 1H), 5.18 (t, J=5.6 Hz, 1H), 4.51 (d, J=5.6 Hz, 2H), 1.35 (s, 3H).

### 8-(3-Bromophenyl)-4-oxo-4H-pyrido[1,2-a]pyrimidine-3-carbaldehyde

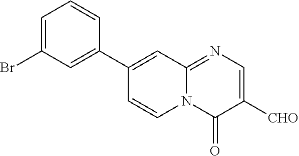 [SMILES: O=Cc1cnc2cc(-c3cccc(Br)c3)ccn2c1=O]

To a solution of 8-(3-bromophenyl)-3-(hydroxymethyl)-4H-pyrido[1,2-a]pyrimidin-4-one (250 mg, 0.75 mmol) in chloroform (20 mL) was added manganese dioxide (666 mg, 7.7 mmol). The mixture was stirred at r.t. for 8 hrs. The mixture was combined with other two batches at 15 mg scale and 16 mg scale. The mixture was filtered through CELITE® and the filtrate was concentrated under reduced pressure. The residue was purified by normal phase flash SiO₂ chromatography (Ethyl acetate/THF/Petroleum ether=1/1/18˜1/1/2) to afford 8-(3-bromophenyl)-4-oxo-4H-pyrido[1,2-a]pyrimidine-3-carbaldehyde (250 mg, 63% yield). m/z: 329 [M⁺], 331 [M+2]⁺ observed.

### 8-(3-Bromophenyl)-3-(((2-hydroxyethyl)amino)methyl)-4H-pyrido[1,2-a]pyrimidin-4-one

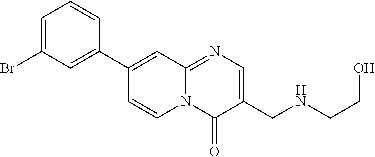 [SMILES: O=c1c(CNCCO)cnc2cc(-c3cccc(Br)c3)ccn12]

To a solution of 8-(3-bromophenyl)-4-oxo-4H-pyrido[1,2-a]pyrimidine-3-carbaldehyde (220 mg, 0.67 mmol) and 2-aminoethanol (0.2 mL, 2.8 mmol) in THF (20 mL) and methanol (20 mL) was added 4A molecular sieve (200 mg) and glacial acetic acid (19 uL, 0.33 mmol). The 10 mixture was stirred at 50° C. for 2 hrs. Then sodium cyanoborohydride (956 mg, 4.5 mmol) was added. The mixture was stirred at 50° C. for 12 hrs. The mixture was diluted with THE (50 mL) and washed with half saturated aqueous brine solution (15 mL), saturated aqueous brine solution (2×15 mL), dried over anhydrous sodium sulfate, filtered and concentrated under reduced pressure to give 8-(3-bromophenyl)-3-(((2-hydroxyethyl)amino)methyl)-4H-pyrido[1,2-a]pyrimidin-4-one as a yellow solid (260 mg, 66% yield) which was used directly. m/z: 374 [M⁺], 376 [M+2]⁺ observed.

### tert-Butyl ((8-(3-bromophenyl)-4-oxo-4H-pyrido[1,2-a]pyrimidin-3-yl)methyl) (2-hydroxyethyl)carbamate

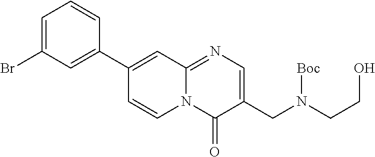 [SMILES: CC(C)(C)OC(=O)N(CCO)Cc1cnc2cc(-c3cccc(Br)c3)ccn2c1=O]

To a solution of 8-(3-bromophenyl)-3-(((2-hydroxyethyl)amino)methyl)-4H-pyrido[1,2-a]pyrimidin-4-one (260 mg, 64% purity, 0.44 mmol) and di-tert-butyl dicarbonate (0.53 mL, 2.3 mmol) in THE (40 mL) and methanol (10 mL) was added TEA (0.3 mL, 2.2 mmol). The mixture was stirred at 10° C. for 1 hr. The mixture was diluted with THE (20 mL). The 25 mixture was washed with half saturated aqueous brine solution (2×20 mL), saturated aqueous brine solution (20 mL), dried over anhydrous sodium sulfate, filtered and concentrated under reduced pressure. The residue was purified by normal phase flash SiO₂ chromatography (ethyl acetate/THF/petroleum ether=1/1/18˜ 2/2/1) to afford tert-butyl ((8-(3-bromophenyl)-4-oxo-4H-pyrido[1,2-a]pyrimidin-3-yl)methyl)(2-hydroxyethyl)carbamate as a yellow solid (159 mg, 33% yield). m/z: 474 [M⁺], 476 [M+2]⁺ observed. ¹H NMR (400 MHz, CDCl₃): δ 9.12 (d, J=7.2 Hz, 1H), 8.60-8.30 (m, 1H), 7.88-7.84 (m, 2H), 7.67-7.64 (m, 2H), 7.45-7.41 (m, 2H), 4.49 (s, 2H), 3.90-3.73 (m, 2H), 3.70-3.50 (m, 2H), 1.48 (s, 9H).

### tert-Butyl ((8-(3′-((2-chloro-5-((5-cyanopyridin-3-yl)methoxy)-4-formylphenoxy) methyl)-2′-methyl-[1,1′-biphenyl]-3-yl)-4-oxo-4H-pyrido[1,2-a]pyrimidin-3-yl)methyl)(2-hydroxyethyl)carbamate

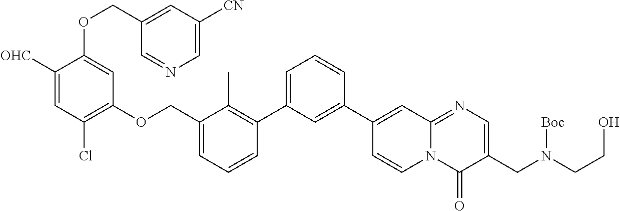 [SMILES: Cc1c(COc2cc(OCc3cncc(C#N)c3)c(C=O)cc2Cl)cccc1-c1cccc(-c2ccn3c(=O)c(CN(CCO)C(=O)OC(C)(C)C)cnc3c2)c1]

To a solution of tert-butyl ((8-(3-bromophenyl)-4-oxo-4H-pyrido[1,2-a]pyrimidin-3-yl) methyl)(2-hydroxyethyl)carbamate (99 mg, 0.2 mmol) and 5-((4-chloro-2-formyl-5-((2-methyl-3-(4,4,5,5-tetramethyl-1,3,2-dioxaborolan-2-yl)benzyl)oxy)phenoxy)methyl)nicotinonitrile (151 mg, 0.3 mmol) in THE (8 mL) and water (2 mL) was added Pd(dppf)Cl₂.CH₂Cl₂ (17 mg, 20.9 umol) and potassium carbonate (74 mg, 0.5 mmol). The mixture was stirred at 80° C. for 3 hrs. The mixture was combined with other three batches at 20 mg scale. To the mixture was added tetrahydrofuran (20 mL) and water (10 mL). The mixture was separated and the aqueous was extracted with THE (15 mL). The organic phase was washed with saturated aqueous brine solution (2×15 mL), dried over anhydrous sodium sulfate, filtered and concentrated under reduced pressure. The residue was purified by normal phase flash SiO₂ chromatography (ethyl acetate/THF/petroleum ether=1/1/18˜ 2/2/1) to afford tert-butyl ((8-(3′-((2-chloro-5-((5-cyanopyridin-3-yl)methoxy)-4-formylphenoxy)methyl)-2′-methyl-[1,1′-biphenyl]-3-yl)-4-oxo-4H-pyrido[1,2-a]pyrimidin-3-yl)methyl)(2-hydroxyethyl)carbamate as a yellow solid (135 mg, 32% yield). m/z: 786 [M+H]⁺ observed.

### tert-Butyl ((8-(3′-((2-chloro-5-((5-cyanopyridin-3-yl)methoxy)-4-(((2-hydroxyethyl)amino)methyl)phenoxy)methyl)-2′-methyl-[1,1′-biphenyl]-3-yl)-4-oxo-4H-pyrido[1,2-a]pyrimidin-3-yl)methyl)(2-hydroxyethyl)carbamate

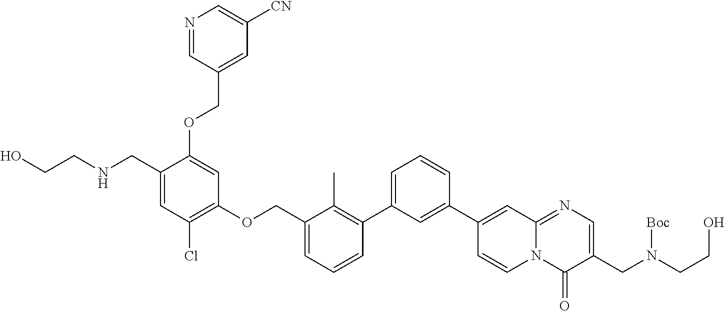 [SMILES: Cc1c(COc2cc(OCc3cncc(C#N)c3)c(CNCCO)cc2Cl)cccc1-c1cccc(-c2ccn3c(=O)c(CN(CCO)C(=O)OC(C)(C)C)cnc3c2)c1]

To a solution of tert-butyl ((8-(3′-((2-chloro-5-((5-cyanopyridin-3-yl)methoxy)-4-formylphenoxy)methyl)-2′-methyl-[1,1′-biphenyl]-3-yl)-4-oxo-4H-pyrido[1,2-a]pyrimidin-3-yl)methyl)(2-hydroxyethyl)carbamate (105 mg, 0.1 mmol) and 2-aminoethanol (84 uL, 1.4 mmol) in methanol (3 mL) and THE (6 mL) was added 4A molecular sieve (80 mg) and glacial acetic acid (21 uL, 0.4 mmol). The mixture was stirred at 70° C. for 2 hrs, cooled to r.t. and sodium cyanoborohydride (63 mg, 1.0 mmol) was added. The mixture was stirred at r.t. for 12 hrs. The mixture was combined with the batch at 15 mg scale. The mixture was filtered through CELITE® and the filter cake was washed with THF (3×5 mL). The filtrate was washed with saturated aqueous brine solution (2×6 mL), dried over anhydrous sodium sulfate, filtered and concentrated under reduced pressure to give tert-butyl ((8-(3′-((2-chloro-5-((5-cyanopyridin-3-yl) methoxy)-4-(((2-hydroxyethyl)amino)methyl)phenoxy)methyl)-2′-methyl-[1,1′-biphenyl]-3-yl)-4-oxo-4H-pyrido[1,2-a]pyrimidin-3-yl)methyl)(2-hydroxyethyl)carbamate as a yellow solid (150 mg, 48% yield) which was used in the next step directly. m/z: 831 [M+H]⁺, 416 [M/2+H]⁺ observed.

### tert-Butyl N-[[8-[3-[3-[[4-[[tert-butoxycarbonyl(2-hydroxyethyl)amino]methyl]-2-chloro-5-[(5-cyano-3-pyridyl)methoxy]phenoxy]methyl]-2-methyl-phenyl]phenyl]-4-oxo-pyrido[1,2-a]pyrimidin-3-yl]methyl]-N-(2-hydroxyethyl)carbamate

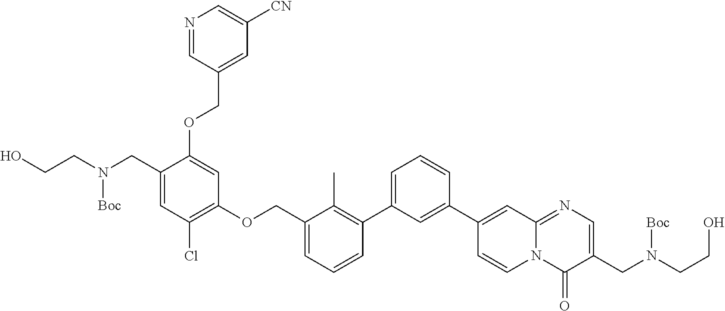 [SMILES: Cc1c(COc2cc(OCc3cncc(C#N)c3)c(CN(CCO)C(=O)OC(C)(C)C)cc2Cl)cccc1-c1cccc(-c2ccn3c(=O)c(CN(CCO)C(=O)OC(C)(C)C)cnc3c2)c1]

To a solution of tert-butyl ((8-(3′-((2-chloro-5-((5-cyanopyridin-3-yl)methoxy)-4- (((2-hydroxyethyl)amino)methyl)phenoxy)methyl)-2′-methyl-[1,1′-biphenyl]-3-yl)-4-oxo-4H-pyrido[1,2-a]pyrimidin-3-yl)methyl)(2-hydroxyethyl)carbamate (150 mg, 0.1 mmol, 57% purity) and di-tert-butyl dicarbonate (0.1 mL, 0.5 mmol) in THE (10 mL) and MeOH (5 mL) was added TEA (0.1 mL, 0.5 mmol). The mixture was stirred at r.t. for 3 hrs. The mixture was diluted with THE (30 mL) and washed with half saturated aqueous brine solution (2×12 mL), saturated aqueous brine solution (12 mL), dried over anhydrous sodium sulfate, filtered and concentrated under reduced pressure. The residue was purified by reverse phase HPLC to give tert-butyl N-[[8-[3-[3-[[4-[[tert-butoxycarbonyl(2-hydroxyethyl)amino]methyl]-2-chloro-5-[(5-cyano-3-pyridyl)methoxy]phenoxy]methyl]-2-methyl-phenyl]phenyl]-4-oxo-pyrido[1,2-a]pyrimidin-3-yl]methyl]-N-(2-hydroxyethyl)carbamate as a light yellow solid (53 mg, 47% yield). m/z: 931 [M+H]⁺ observed.

### 5-((4-Chloro-2-(((2-hydroxyethyl)amino)methyl)-5-((3′-(3-(((2-hydroxyethyl)amino)methyl)-4-oxo-4H-pyrido[1,2-a]pyrimidin-8-yl)-2-methyl-[1,1′-biphenyl]-3-yl)methoxy)phenoxy)methyl)nicotinamide

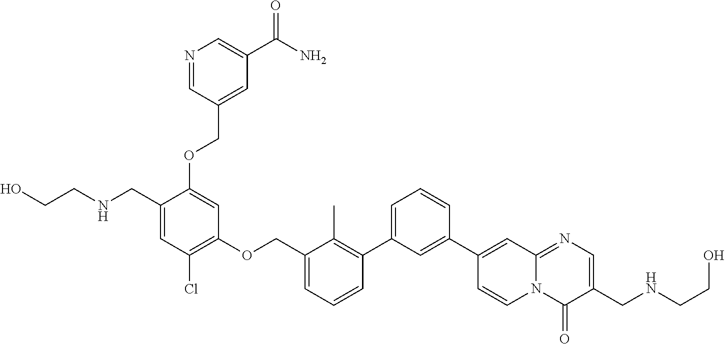 [SMILES: Cc1c(COc2cc(OCc3cncc(C(N)=O)c3)c(CNCCO)cc2Cl)cccc1-c1cccc(-c2ccn3c(=O)c(CNCCO)cnc3c2)c1]

To a solution of tert-butyl N-[[8-[3-[3-[[4-[[tert-butoxycarbonyl(2-hydroxyethyl) amino]methyl]-2-chloro-5-[(5-cyano-3-pyridyl)methoxy]phenoxy]methyl]-2-methyl-phenyl]phenyl]-4-oxo-pyrido[1,2-a]pyrimidin-3-yl]methyl]-N-(2-hydroxyethyl)carbamate (50 mg, 21.5 umol) and in THE (4 mL) at 0° C. was added 4 N HCl solution in EtOAc (6 mL) dropwise. The mixture was stirred at 0° C. for 2 hrs and at 20° C. for 12 hrs. The mixture was concentrated under reduced pressure and the residue was neutralized with saturated aqueous sodium bicarbonate solution. Tetrahydrofuran (30 mL) was added and the mixture was separated. The organic phase was washed with saturated aqueous brine solution (5 mL), dried over anhydrous sodium sulfate, filtered and concentrated under reduced pressure. The residue was purified by reverse phase HPLC to afford 5-((4-chloro-2-(((2-hydroxyethyl)amino) methyl)-5-((3′-(3-(((2-hydroxyethyl)amino)methyl)-4-oxo-4H-pyrido[1,2-a]pyrimidin-8-yl)-2-methyl-[1,1′-biphenyl]-3-yl)methoxy)phenoxy)methyl)nicotinamide (5.3 mg, 30% yield). m/z: 749 [M+H]⁺ observed. ¹H NMR (400 MHz, CD₃OD): δ9.12 (d, J=7.6 Hz, 1H), 9.00 (d, J=2.4 Hz, 1H), 8.81 (d, J=2.0 Hz, 1H), 8.41 (s, 1H), 8.39 (s, 1H), 7.95 (d, J=1.6 Hz, 1H), 7.91 (br d, J=8.0 Hz, 1H), 7.83 (s, 1H), 7.78 (dd, J=7.6, 2.0 Hz, 1H), 7.67 (t, J=7.6 Hz, 1H), 7.56-7.45 (m, 2H), 7.35 (s, 1H), 7.32-7.27 (m, 2H), 6.99 (s, 1H), 5.33 (s, 2H), 5.28 (s, 2H), 3.88 (s, 2H), 3.81 (s, 2H), 3.72-3.69 (m, 2H), 3.67-3.64 (m, 2H), 2.81-2.78 (m, 2H), 2.75-2.72 (m, 2H), 2.33 (s, 3H).

### Example 2: 5-(((2-Hydroxyethyl)amino)methyl)-N-(3′-(3-(((2-hydroxyethyl)amino)methyl)-4-oxo-4H-pyrido[1,2-a]pyrimidin-8-yl)-2-methyl-[1,1′-biphenyl]-3-yl)picolinamide

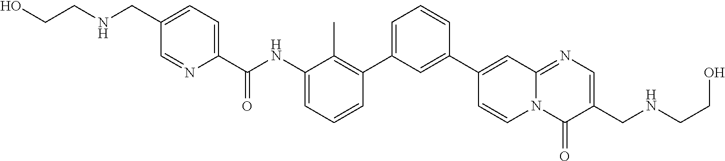 [SMILES: Cc1c(NC(=O)c2ccc(CNCCO)cn2)cccc1-c1cccc(-c2ccn3c(=O)c(CNCCO)cnc3c2)c1]

### tert-Butyl ((8-(3′-amino-2′-methyl-[1,1′-biphenyl]-3-yl)-4-oxo-4H-pyrido[1,2-a]pyrimidin-3-yl)methyl)(2-hydroxyethyl)carbamate

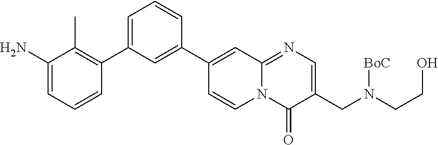

To a solution of tert-butyl((8-(3-bromophenyl)-4-oxo-4H-pyrido[1,2-a]pyrimidin-3-yl)methyl)(2-hydroxyethyl)carbamate (269 mg, 0.57 mmol) and 2-methyl-3-(4,4,5,5-tetramethyl-1,3,2-dioxaborolan-2-yl)aniline (259 mg, 1.1 mmol) in THE (15 mL) and water (5 mL) was added potassium carbonate (238 mg, 1.7 mmol) and ditert-butyl(cyclopentyl)phosphane-dichloropalladium-iron (46.6 mg, 0.071 mmol). The mixture was stirred at 80° C. for 12 hrs. The mixture was combined with the batch at 20 mg scale. The mixture was filtered through CELITE® and the filtrate was diluted with THE (30 mL), washed with saturated aqueous brine solution (2×20 mL), dried over anhydrous sodium sulfate, filtered and concentrated under reduced pressure. The residue was purified by normal phase flash SiO₂ chromatography (90-17% petroleum ether/40% ethyl acetate in THF) to afford tert-butyl((8-(3′-amino-2′-methyl-[1,1′-biphenyl]-3-yl)-4-oxo-4H-pyrido[1,2-a]pyrimidin-3-yl)methyl)(2-hydroxyethyl)carbamate (264 mg, 86% yield, m/z: 501 [M+H]⁺ observed) as a yellow solid.

### tert-Butyl N-[[8-[3-(3-amino-2-methyl-phenyl)phenyl]-4-oxo-pyrido[1,2-a]pyrimidin-3-yl]methyl]-N-(2-hydroxyethyl)carbamate

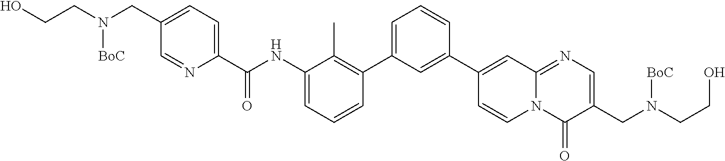

To a solution of tert-butyl ((8-(3′-amino-2′-methyl-[1,1′-biphenyl]-3-yl)-4-oxo-4H-pyrido[1,2-a]pyrimidin-3-yl)methyl)(2-hydroxyethyl)carbamate (244 mg, 0.49 mmol) and lithium 5-(((tert-butoxycarbonyl)(2-hydroxyethyl)amino)methyl)picolinate (300 mg, 0.99 mmol) in DMF (10 mL) was added 2-chloro-1,3-dimethyl-4,5-dihydroimidazol-1-ium;chloride (244 mg, 1.4 mmol) and DIPEA (0.5 mL, 2.9 mmol). The mixture was stirred at r.t. for 12 hrs.

The mixture was combined with two batches at 10 mg scale. The mixture was diluted with THE (50 mL) and washed with saturated aqueous brine solution (2×15 mL), dried over anhydrous sodium sulfate, filtered and concentrated under reduced pressure. The residue was purified by normal phase flash SiO₂ chromatography (90-20% petroleum ether/50% THE in ethyl acetate) to give two batches of tert-butyl N-[[8-[3-(3-amino-2-methyl-phenyl)phenyl]-4-oxo-pyrido[1,2-a]pyrimidin-3-yl]methyl]-N-(2-hydroxyethyl)carbamate: batch 1 (140 mg, m/z: 779 [M+H]⁺, 390 [M/2+H]⁺ observed); batch 2 (89 mg, m/z: 779 [M+H]⁺, 390 [M½+H]⁺ observed) as yellow solid. The total yield is 26%.

### 5-(((2-hHydroxyethyl)amino)methyl)-N-(3′-(3-(((2-hydroxyethyl)amino)methyl)-4-oxo-4H-pyrido[1,2-a]pyrimidin-8-yl)-2-methyl-[1,1′-biphenyl]-3-yl)picolinamide

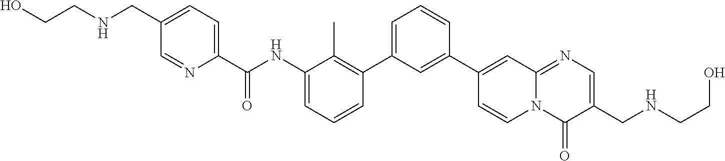 [SMILES: Cc1c(NC(=O)c2ccc(CNCCO)cn2)cccc1-c1cccc(-c2ccn3c(=O)c(CNCCO)cnc3c2)c1]

To a solution of tert-butyl N-[[6-[[3-[3-[3-[[tert-butoxycarbonyl(2-hydroxyethyl) amino]methyl]-4-oxo-pyrido[1,2-a]pyrimidin-8-yl]phenyl]-2-methyl-phenyl]carbamoyl]-3-pyridyl]methyl]-N-(2-hydroxyethyl)carbamate (130 mg, 0.17 mmol) in THF (5 mL) was added HCl (4 M solution in EtOAc, 6 mL, 24 mmol) dropwise. The mixture was stirred at r.t. for 12 hrs. The mixture was combined with the batch at 10 mg scale. The mixture was concentrated under reduced pressure. The residue was purified by reverse phase HPLC to afford 5-(((2-hydroxyethyl)amino)methyl)-N-(3′-(3-(((2-hydroxyethyl)amino)methyl)-4-oxo-4H-pyrido[1,2-a]pyrimidin-8-yl)-2-methyl-[1,1′-biphenyl]-3-yl)picolinamide as a hydrochloride salt (41.2 mg, 37% yield, m/z: 579 [M+H]⁺, 290 [M/2+H]⁺ observed) as a yellow solid. ¹H NMR (400 MHz, CD₃OD): δ9.38 (d, J=7.2 Hz, 1H), 8.89 (s, 1H), 8.71 (s, 1H), 8.36-8.28 (m, 3H), 8.28-8.21 (m, 1H), 8.09 (br d, J=8.0 Hz, 1H), 8.04 (s, 1H), 7.84 (d, J=8.0 Hz, 1H), 7.78 (t, J=7.6 Hz, 1H), 7.70 (d, J=7.6 Hz, 1H), 7.40 (t, J=8.0 Hz, 1H), 7.27 (d, J=7.6 Hz, 1H), 4.46 (s, 2H), 4.39 (s, 2H), 3.94-3.83 (m, 4H), 3.28-3.22 (m, 2H), 2.29 (s, 3H).

### Example 3: 5-((4-Chloro-2-(((2-hydroxyethyl)amino)methyl)-5-((3′-(3-(((2-hydroxyethyl) amino)methyl)-4-oxo-4H-pyrido[1,2-a]pyrimidin-8-yl)-2,2′-dimethyl-[1,1′-biphenyl]-3-yl)methoxy)phenoxy)methyl)nicotinonitrile

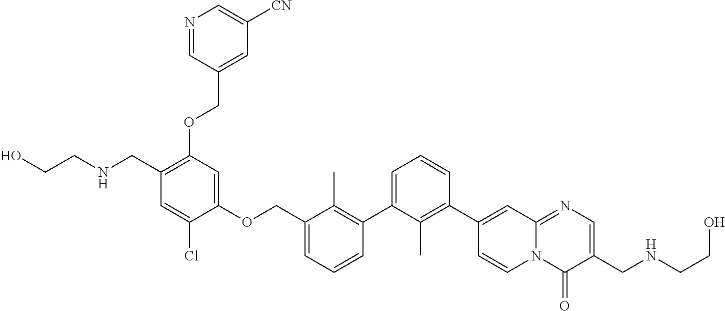 [SMILES: Cc1c(COc2cc(OCc3cncc(C#N)c3)c(CNCCO)cc2Cl)cccc1-c1cccc(-c2ccn3c(=O)c(CNCCO)cnc3c2)c1C]

### Ethyl 8-(3-bromo-2-methylphenyl)-4-oxo-4H-pyrido[1,2-a]pyrimidine-3-carboxylate

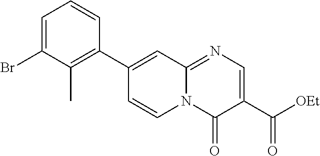 [SMILES: CCOC(=O)c1cnc2cc(-c3cccc(Br)c3C)ccn2c1=O]

To a solution of ethyl 8-bromo-4-oxo-4H-pyrido[1,2-a]pyrimidine-3-carboxylate (7 g, 23.6 mmol) and (3-bromo-2-methylphenyl)boronic acid (5.2 g, 24.4 mmol) in water (30 mL) and dioxane (190 mL) was added potassium carbonate (7 g, 51 mmol) and Pd(dppf)Cl₂.CH₂Cl₂ (1.9 g, 2.4 mmol). The mixture was stirred at 80° C. for 3 hrs under nitrogen gas. The mixture was combined with the batch at 100 mg scale. The mixture was concentrated. To the residue was added dichloromethane (150 mL) and water (50 mL). The mixture was filtered through CELITE® and washed with dichloromethane (3×20 mL). The filtrate was separated. The organic phase was washed with brine (2×70 mL), dried over anhydrous sodium sulfate, filtered and concentrated under reduced pressure. The residue was purified by normal phase flash SiO₂ chromatography (90-20% petroleum ether/50% THE in ethyl acetate) to give 6.5 g semi-purified product. The solid was triturated with THF/EA/petroleum ether (10 mL/10 mL/10 mL) at r.t. for 15 min. The resulting yellow solid was collected by filtration to afford ethyl 8-(3-bromo-2-methylphenyl)-4-oxo-4H-pyrido[1,2-a] pyrimidine-3-carboxylate (5.5 g, 59%) as a yellow solid. ¹H NMR (400 MHz, CDCl₃) δ 9.31 (d, J=7.2 Hz, 1H), 9.09 (s, 1H), 7.73-7.70 (m, 2H), 7.30-7.21 (m, 3H), 4.48-4.43 (m, 2H), 2.41 (s, 3H), 1.46-1.43 (m, 3H).

### 8-(3-Bromo-2-methylphenyl)-3-(hydroxymethyl)-4H-pyrido[1,2-a]pyrimidin-4-one

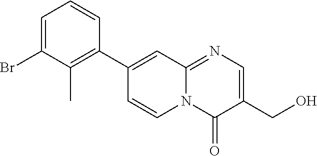 [SMILES: Cc1c(Br)cccc1-c1ccn2c(=O)c(CO)cnc2c1]

To a solution of ethyl 8-(3-bromo-2-methylphenyl)-4-oxo-4H-pyrido[1,2-a] pyrimidine-3-carboxylate (5 g, 12.9 mmol) in dichloromethane (120 mL) and ether (120 mL) under nitrogen gas was added diisobutylaluminum hydride (1 M solution in toluene, 30 mL, 30 mmol) dropwise at −10° C. (over 15 min). The mixture was stirred at −10° C. for 1 hr, warmed to r.t. and stirred for 5 hrs. The mixture was cooled to 0° C., quenched carefully with 1 N aqueous HCl solution (40 mL) and a lot of yellow solid was precipitated. The suspension was filtered and washed with water (2×20 mL) and dried under vacuum to give 3.3 g of a yellow solid. The solid was purified by normal phase SiO₂ chromatography (90-33% petroleum ether/50% THE in ethyl acetate) to afford the product (0.2 g) as a yellow solid. The filtrate was separated and the organic phase was washed with saturated aqueous brine solution (2×30 mL), dried over anhydrous sodium sulfate, filtered and concentrated under reduced pressure. The residue was purified by normal phase flash SiO₂ chromatography (90-33% petroleum ether/50% THE in ethyl acetate) to afford 1.6 g of semi-purified product as a yellow solid. The solid was triturated with petroleum ether/EtOAc (2 mL/10 mL) at r.t. for 15 min. The resulting yellow solid was collected by filtration to offer 8-(3-bromo-2-methylphenyl)-3-(hydroxymethyl)-4H-pyrido[1,2-a]pyrimidin-4-one (1.3 g, m/z: 345 [M⁺], 347 [M+2]⁺ observed) as a yellow solid. 1.5 g of the product was obtained and the total yield is 34%.

### 8-(3-Bromo-2-methylphenyl)-4-oxo-4H-pyrido[1,2-a]pyrimidine-3-carbaldehyde

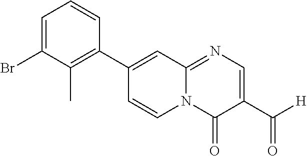 [SMILES: Cc1c(Br)cccc1-c1ccn2c(=O)c(C=O)cnc2c1]

To a solution of 8-(3-bromo-2-methylphenyl)-3-(hydroxymethyl)-4H-pyrido[1,2-a]pyrimidin-4-one (1.3 g, 3.7 mmol) in chloroform (140 mL) was added manganese dioxide (3.3 g, 38 mmol). The mixture was stirred at 40° C. for 12 hrs. The mixture was combined with the batch at 100 mg scale. The mixture was filtered through CELITE® and the filtrate was concentrated under reduced pressure. The residue was purified by normal phase flash SiO₂ chromatography (90-50% petroleum ether/50% THF in ethyl acetate) to afford 8-(3-bromo-2-methylphenyl)-4-oxo-4H-pyrido[1,2-a] pyrimidine-3-carbaldehyde (0.8 g, 58%) as a yellow solid. ¹H NMR (400 MHz, CDCl₃) δ 10.42 (s, 1H), 9.32 (d, J=7.2 Hz, 1H), 8.94 (s, 1H), 7.76-7.75 (m, 2H), 7.37 (d, J=5.6 Hz, 1H), 7.26-7.24 (m, 2H), 2.42 (s, 3H).

### 8-(3-Bromo-2-methylphenyl)-3-(((2-hydroxyethyl)amino)methyl)-4H-pyrido[1,2-a]pyrimidin-4-one

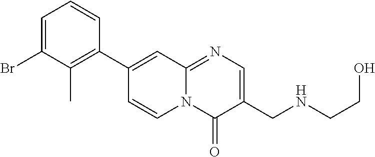 [SMILES: Cc1c(Br)cccc1-c1ccn2c(=O)c(CNCCO)cnc2c1]

To a solution of 8-(3-bromo-2-methylphenyl)-4-oxo-4H-pyrido[1,2-a]pyrimidine-3-carbaldehyde (0.8 g, 2.3 mmol) and 2-aminoethanol (1 mL, 16.5 mmol) in THE (35 mL) and methanol (18 mL) was added 4 A molecular sieve (2 g) and glacial acetic acid (0.8 mL, 14 mmol). The mixture was stirred at 50° C. for 12 hrs. Sodium cyanoborohydride (0.6 g, 9.6 mmol) was added and the mixture was stirred at r.t. for 8 hrs. The mixture was diluted with THE (40 mL) and washed with half saturated aqueous brine solution (25 mL), saturated aqueous brine solution (2×25 mL), dried over anhydrous sodium sulfate, filtered and concentrated under reduced pressure to give 8-(3-bromo-2-methylphenyl)-3-(((2-hydroxyethyl)amino)methyl)-4H-pyrido[1,2-a]pyrimidin-4-one (0.9 g, 81% yield, m/z: 388 [M⁺], 390 [M+2]⁺ observed) which was used directly.

### tert-Butyl ((8-(3-bromo-2-methylphenyl)-4-oxo-4H-pyrido[1,2-a]pyrimidin-3-yl) methyl)(2-hydroxyethyl)carbamate

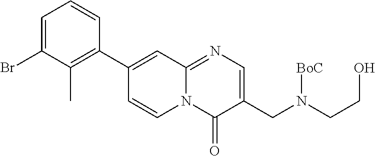

To a solution of 8-(3-bromo-2-methylphenyl)-3-(((2-hydroxyethyl)amino)methyl)-4H-pyrido[1,2-a]pyrimidin-4-one (0.9 g, 1.9 mmol) and di-tert-butyl dicarbonate (1.6 mL, 7.0 mmol) in THF (15 mL) and MeOH (10 mL) was added TEA (1.1 mL, 7.6 mmol). The mixture was stirred at r.t. for 4 hrs. The mixture was diluted with THF (40 mL). The mixture was washed with half saturated aqueous brine solution (2×15 mL), saturated aqueous brine solution (20 mL), dried over anhydrous sodium sulfate, filtered and concentrated under reduced pressure. The residue was purified by normal phase flash SiO₂ chromatography (90-17% petroleum ether/50% THF in ethyl acetate) twice to give 0.5 g of a yellow solid which contains some amount of TEA. The solid was dissolved in THE (25 mL) and the solution was washed with half saturated aqueous brine solution (4×10 mL), saturated aqueous brine solution (2×10 mL), dried over anhydrous sodium sulfate, filtered and concentrated under reduced pressure to afford tert-butyl ((8-(3-bromo-2-methylphenyl)-4-oxo-4H-pyrido[1,2-a]pyrimidin-3-yl)methyl)(2-hydroxyethyl)carbamate (380 mg, 41% yield, m/z: 488 [M⁺], 490 [M+2]⁺ observed) as a yellow solid. ¹H NMR (400 MHz, CDCl₃) δ 9.11 (d, J=7.2 Hz, 1H), 8.45-8.39 (m, 1H), 7.70 (d, J=7.2 Hz, 1H), 7.60 (s, 1H), 7.26-7.17 (m, 3H), 4.52 (s, 2H), 3.83 (s, 2H), 3.64 (br s, 2H), 2.40 (s, 3H), 1.49 (s, 9H).

### tert-Butyl ((8-(3′-((2-chloro-5-((5-cyanopyridin-3-yl)methoxy)-4-formylphenoxy)methyl)-2,2′-dimethyl-[1,1′-biphenyl]-3-yl)-4-oxo-4H-pyrido[1,2-a]pyrimidin-3-yl)methyl)(2-hydroxyethyl)carbamate

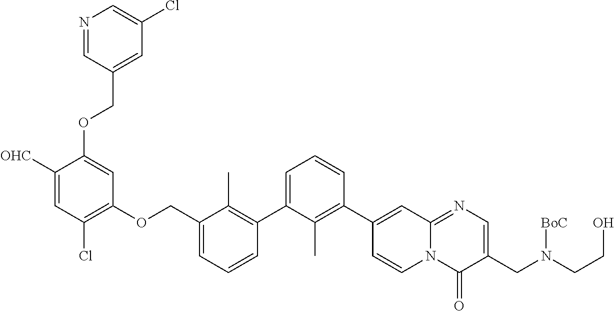

To a solution of tert-butyl ((8-(3-bromo-2-methylphenyl)-4-oxo-4H-pyrido[1,2-a] pyrimidin-3-yl)methyl)(2-hydroxyethyl)carbamate (350 mg, 0.73 mmol) and 5-((4-chloro-2-formyl-5-((2-methyl-3-(4,4,5,5-tetramethyl-1,3,2-dioxaborolan-2-yl)benzyl)oxy)phenoxy)methyl)nicotinonitrile (410 mg, 0.79 mmol) in THE (20 mL) and water (4 mL) was added Pd(dppf)Cl₂.CH₂Cl₂ (85 mg, 0.1 mmol) and potassium carbonate (300 mg, 2.2 mmol). The mixture was stirred at 80° C. for 3 hrs. To the mixture was added THE (20 mL) and water (10 mL). The mixture was separated and the aqueous phase was extracted with THE (15 mL). The organic phase was washed with saturated aqueous brine solution (2×15 mL), dried over anhydrous sodium sulfate, filtered and concentrated under reduced pressure. The residue was purified by normal phase flash SiO₂ chromatography (90-20% petroleum ether/50% THF in ethyl acetate) to afford tert-butyl ((8-(3′-((2-chloro-5-((5-cyanopyridin-3-yl)methoxy)-4- formylphenoxy)methyl)-2,2′-dimethyl-[1,1′-biphenyl]-3-yl)-4-oxo-4H-pyrido[1,2-a]pyrimidin-3-yl)methyl)(2-hydroxyethyl)carbamate as a yellow solid (390 mg, 39% yield). m/z: 800 [M+H]⁺ observed.

### tert-Butyl ((8-(3′-((2-chloro-5-((5-cyanopyridin-3-yl)methoxy)-4-(((2-hydroxyethyl)amino)methyl)phenoxy)methyl)-2,2′-dimethyl-[1,1′-biphenyl]-3-yl)-4-oxo-4H-pyrido[1,2-a]pyrimidin-3-yl)methyl)(2-hydroxyethyl)carbamate

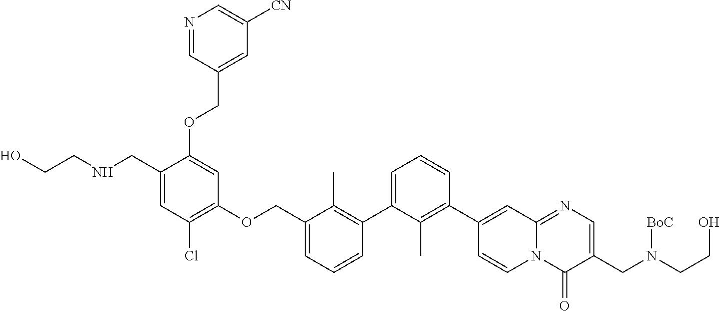

To a solution of tert-butyl ((8-(3′-((2-chloro-5-((5-cyanopyridin-3-yl)methoxy)-4-formylphenoxy)methyl)-2,2′-dimethyl-[1,1′-biphenyl]-3-yl)-4-oxo-4H-pyrido[1,2-a]pyrimidin-3-yl)methyl)(2-hydroxyethyl)carbamate (390 mg, 0.49 mmol) and 2-aminoethanol (0.2 mL, 3.4 mmol) in THE (20 mL) and methanol (10 mL) was added 4 A molecular sieve (400 mg) and glacial acetic acid (0.17 mL, 2.9 mmol). The mixture was stirred at 50° C. for 12 hrs, sodium cyanoborohydride (156 mg, 2.5 mmol) was added. The mixture was stirred at r.t. for 5 hrs. The mixture was combined with the batch at 25 mg scale. The mixture was diluted with THE (30 mL) and washed with half saturated aqueous brine solution (15 mL), saturated aqueous brine solution (2×15 mL), dried over anhydrous sodium sulfate, filtered and concentrated under reduced pressure to give the crude product (600 mg, 38% purity) as a yellow solid. 320 mg of the crude was purified by reverse phase to afford two batches of tert-butyl ((8-(3′-((2-chloro-5-((5-cyanopyridin-3-yl)methoxy)-4-(((2-hydroxyethyl)amino) methyl)phenoxy)methyl)-2,2′-dimethyl-[1,1′-biphenyl]-3-yl)-4-oxo-4H-pyrido[1,2-a]pyrimidin-3-yl)methyl)(2-hydroxyethyl)carbamate: batch 1: (90 mg, 71% purity, m/z: 845 [M+H]⁺, 423 [M/2+H]⁺ observed); batch 2: (280 mg, 38% purity, m/z: 845 [M+H]⁺, 423 [M/2+H]⁺ observed) as yellow solid which were used directly in the next step and the step of Part A. The total yield is 41%.

### tert-butyl N-[[8-[3-[3-[[4-[[tert-butoxycarbonyl(2-hydroxyethyl)amino]methyl]-2-chloro-5-[(5-cyano-3-pyridyl)methoxy]phenoxy]methyl]-2-methyl-phenyl]-2-methyl-phenyl]-4-oxo-pyrido[1,2-a]pyrimidin-3-yl]methyl]-N-(2-hydroxyethyl)carbamate

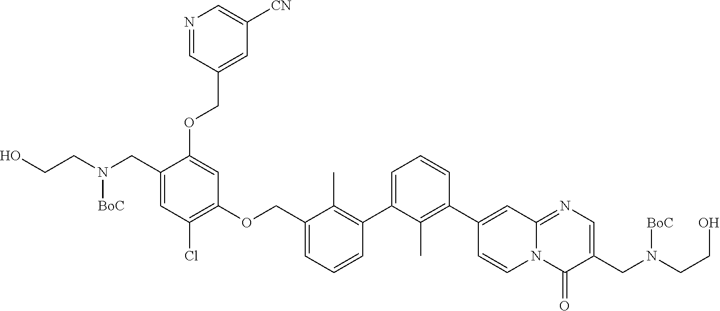

To a solution of tert-butyl ((8-(3′-((2-chloro-5-((5-cyanopyridin-3-yl)methoxy)-4- (((2-hydroxyethyl)amino)methyl)phenoxy)methyl)-2,2′-dimethyl-[1,1′-biphenyl]-3-yl)-4-oxo-4H-pyrido[1,2-a]pyrimidin-3-yl)methyl)(2-hydroxyethyl)carbamate (280 mg, 0.12 mmol, 38% purity) in THE (5 mL) and methanol (3 mL) was added di-tert-butyl dicarbonate (0.046 mL, 0.2 mmol) and TEA (0.065 mL, 0.47 mmol). The mixture was stirred at r.t. for 3 hrs. The mixture was combined with the batch at 25 mg scale. The mixture was diluted with THE (20 mL) and washed with half saturated aqueous brine solution (10 mL), saturated aqueous brine solution (2×10 mL), dried over anhydrous sodium sulfate, filtered and concentrated under reduced pressure. The residue was purified by normal phase flash SiO₂ chromatography (90˜20% petroleum ether/50% THF in ethyl acetate) to give tert-butyl N-[[8-[3-[3-[[4-[[tert-butoxycarbonyl (2-hydroxyethyl)amino]methyl]-2-chloro-5-[(5-cyano-3-pyridyl)methoxy]phenoxy]methyl]-2-methyl-phenyl]-2-methyl-phenyl]-4-oxo-pyrido[1,2-a]pyrimidin-3-yl]methyl]-N-(2-hydroxyethyl)carbamate (233 mg, 67% yield, 41% purity, m/z: 945 [M+H]⁺ observed) as a yellow solid which was used directly (see the next step: Part B).

### 5-((4-Chloro-2-(((2-hydroxyethyl)amino)methyl)-5-((3′-(3-(((2-hydroxyethyl)amino)methyl)-4-oxo-4H-pyrido[1,2-a]pyrimidin-8-yl)-2,2′-dimethyl-[1,1′-biphenyl]-3-yl)methoxy)phenoxy)methyl)nicotinonitrile

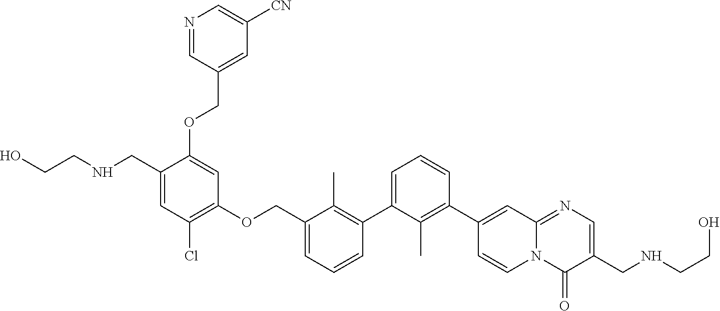 [SMILES: Cc1c(COc2cc(OCc3cncc(C#N)c3)c(CNCCO)cc2Cl)cccc1-c1cccc(-c2ccn3c(=O)c(CNCCO)cnc3c2)c1C]

To a solution of tert-butyl N-[[8-[3-[3-[[2-chloro-5-[(5-cyano-3-pyridyl)methoxy]-4-[(2-hydroxyethylamino)methyl]phenoxy]methyl]-2-methyl-phenyl]-2-methyl-phenyl]-4-oxo-pyrido[1,2-a]pyrimidin-3-yl]methyl]-N-(2-hydroxyethyl)carbamate (90 mg, 0.11 mmol) in THE (5 mL) at 0° C. was added HCl (4 M solution in EtOAc, 1.5 mL, 6 mmol). The mixture was stirred at 0° C. for 2 hrs allowed its temperature warm to 25° C. The mixture was combined with the batch at 10 mg scale. The mixture was concentrated at 30° C. to give a yellow solid. To the solid was added THE (20 mL) and the mixture was neutralized with saturated aqueous sodium bicarbonate solution. The mixture was separated and the aqueous phase was extracted with THE (2×10 mL). The combined organic phase was washed with saturated aqueous brine solution (2×5 mL), dried over anhydrous sodium sulfate, filtered and concentrated under reduced pressure to give a yellow solid (50 mg).

### Part B:

To a solution of tert-butyl N-[[8-[3-[3-[[4-[[tert-butoxycarbonyl(2-hydroxyethyl) amino]methyl]-2-chloro-5-[(5-cyano-3-pyridyl)methoxy]phenoxy]methyl]-2-methyl-phenyl]-2-methyl-phenyl]-4-oxo-pyrido[1,2-a]pyrimidin-3-yl]methyl]-N-(2-hydroxyethyl)carbamate (233 mg, 0.1 mmol, 41% purity) in THE (4 mL) at 0° C. was added HCl (4 M solution in 20 EtOAc, 1 mL, 4 mmol). The mixture was stirred for 12 hrs allowed its temperature warm to r.t. The mixture was concentrated at 30° C. to give a yellow solid. The solid was neutralized with saturated aqueous sodium bicarbonate solution (10 mL) and the suspension was extracted with THE (2×20 mL). The combined organic phase was washed with saturated aqueous brine solution (2×5 mL), dried over anhydrous sodium sulfate, filtered and concentrated under reduced pressure to give the crude product (100 mg) as a yellow solid. The above two batches of yellow solid were combined and purified by reverse phase HPLC to afford 5-((4-chloro-2-(((2-hydroxyethyl)amino)methyl)-5- ((3′-(3-(((2-hydroxyethyl)amino)methyl)-4-oxo-4H-pyrido[1,2-a]pyrimidin-8-yl)-2,2′-dimethyl-[1,1′-biphenyl]-3-yl)methoxy)phenoxy)methyl)nicotinonitrile was obtained as a light yellow solid (36.4 mg, 28%). m/z: 745 [M+H]⁺, 373 [M/2+H]⁺ observed. ¹H NMR (400 MHz, CD₃OD): δ9.12 (d, J=8.0 Hz, 1H), 8.94 (d, J=2.0 Hz, 1H), 8.90 (d, J=2.0 Hz, 1H), 8.40 (s, 1H), 8.34 (t, J=2.0 Hz, 1H), 7.65 (d, J=1.2 Hz, 1H), 7.50 (d, J=6.8 Hz, 1H), 7.47-7.38 (m, 3H), 7.34 (s, 1H), 7.32-7.24 (m, 2H), 7.19-7.15 (m, 1H), 6.98 (s, 1H), 5.30 (s, 2H), 5.28 (s, 2H), 3.87 (s, 2H), 3.77 (s, 2H), 3.73-3.67 (m, 2H), 3.66-3.61 (m, 2H), 2.79 (t, J=5.6 Hz, 2H), 2.70 (t, J=5.6 Hz, 2H), 2.16 (s, 3H), 2.02 (s, 3H).

### Example 4: 5-((4-Chloro-2-(((2-hydroxyethyl)amino)methyl)-5-((3′-(3-(((2-hydroxyethyl)amino)methyl)-4-oxo-4H-pyrido[1,2-a]pyrimidin-8-yl)-2-methyl-[1,1′-biphenyl]-3-yl)methoxy)phenoxy)methyl)nicotinonitrile

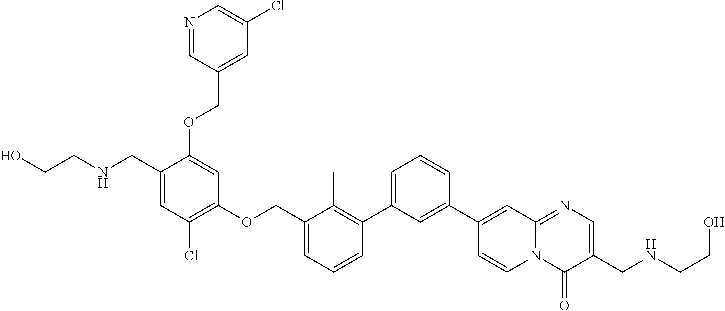 [SMILES: Cc1c(COc2cc(OCc3cncc(Cl)c3)c(CNCCO)cc2Cl)cccc1-c1cccc(-c2ccn3c(=O)c(CNCCO)cnc3c2)c1]

### tert-Butyl ((8-(3′-((2-chloro-5-((5-cyanopyridin-3-yl)methoxy)-4-formylphenoxy) methyl)-2′-methyl-[1,1′-biphenyl]-3-yl)-4-oxo-4H-pyrido[1,2-a]pyrimidin-3-yl)methyl)(2-hydroxyethyl)carbamate

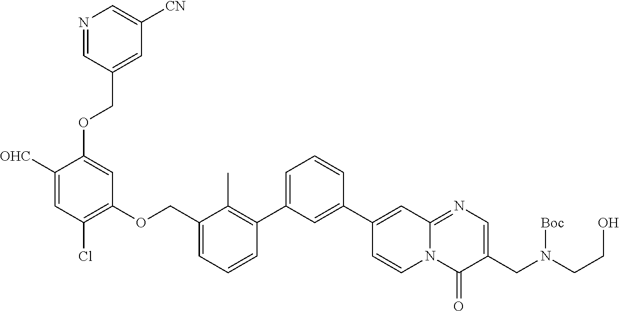 [SMILES: Cc1c(COc2cc(OCc3cncc(C#N)c3)c(C=O)cc2Cl)cccc1-c1cccc(-c2ccn3c(=O)c(CN(CCO)C(=O)OC(C)(C)C)cnc3c2)c1]

To a solution of tert-butyl ((8-(3-bromophenyl)-4-oxo-4H-pyrido[1,2-a]pyrimidin-3-yl) methyl)(2-hydroxyethyl)carbamate (500 mg, 1.0 mmol) and 5-((4-chloro-2-formyl-5-((2-methyl-3-(4,4,5,5-tetramethyl-1,3,2-dioxaborolan-2-yl)benzyl)oxy)phenoxy)methyl) nicotinonitrile (710 mg, 1.4 mmol) in water (5 mL) and THF (20 mL) was added ditert-butyl(cyclopentyl)phosphane;dichloropalladium;iron (100 mg, 0.15 mmol) and potassium carbonate (400 mg, 2.9 mmol) under nitrogen gas. The mixture was stirred at 80° C. for 4 hrs. To the mixture was added THE (20 mL) and water (15 mL). The organic phase was washed with saturated aqueous brine solution (2×20 mL), dried over anhydrous sodium sulfate, filtered and concentrated under reduced pressure. The residue was purified by normal phase flash SiO₂ chromatography (ethyl acetate/THF/petroleum ether=1/1/18˜ 2/2/1) to afford tert-butyl ((8-(3′-((2-chloro-5-((5-cyanopyridin-3-yl)methoxy)-4-formylphenoxy)methyl)-2′-methyl-[1,1′-biphenyl]-3-yl)-4-oxo-4H-pyrido[1,2-a]pyrimidin-3-yl)methyl)(2-hydroxyethyl)carbamate (780 mg, 64% yield, m/z: 786 [M+H]⁺ observed) as a yellow solid. ¹H NMR (400 MHz, DMSO-d₆): δ 10.24 (s, 1H), 9.04-9.02 (m, 2H), 9.01 (d, J=7.2 Hz, 1H), 8.56 (s, 1H), 8.25 (s, 1H), 8.09 (br s, 1H), 8.00 (m, 1H), 7.91 (s, 1H), 7.90-7.85 (m, 1H), 7.74 (s, 1H), 7.70-7.65 (m, 1H), 7.60-7.55 (m, 1H), 7.53-7.49 (m, 1H), 7.39-7.33 (m, 2H), 7.29 (s, 1H), 5.50 (s, 2H), 5.45 (s, 2H), 4.38 (s, 2H), 3.53-3.47 (m, 2H), 2.31 (s, 3H), 1.37 (s, 9H).

### tert-Butyl ((8-(3′-((2-chloro-5-((5-cyanopyridin-3-yl)methoxy)-4-(((2-hydroxyethyl) amino)methyl)phenoxy)methyl)-2′-methyl-[1,1′-biphenyl]-3-yl)-4-oxo-4H-pyrido[1,2-a]pyrimidin-3-yl)methyl)(2-hydroxyethyl)carbamate

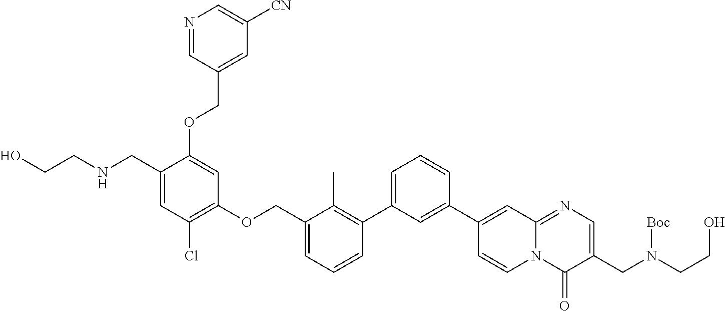 [SMILES: Cc1c(COc2cc(OCc3cncc(C#N)c3)c(CNCCO)cc2Cl)cccc1-c1cccc(-c2ccn3c(=O)c(CN(CCO)C(=O)OC(C)(C)C)cnc3c2)c1]

To a solution of tert-butyl ((8-(3′-((2-chloro-5-((5-cyanopyridin-3-yl)methoxy)-4-formylphenoxy)methyl)-2′-methyl-[1,1′-biphenyl]-3-yl)-4-oxo-4H-pyrido[1,2-a]pyrimidin-3-yl)methyl)(2-hydroxyethyl)carbamate (780 mg, 0.99 mmol) and 2-aminoethanol (0.5 mL, 8.6 mmol) in methanol (10 mL) and THF (20 mL) was added 4A MS (500 mg) and glacial acetic acid (0.3 mL, 5.2 mmol). The mixture was stirred at 70° C. for 3 hrs. Then sodium cyanoborohydride (200 mg, 3.2 mmol) was added. The mixture was stirred at r.t. for 5 hrs.

The mixture was diluted with THE (40 mL), washed with half saturated aqueous brine solution (2×40 mL), saturated aqueous brine solution (2×40 mL), dried over anhydrous sodium sulfate, filtered and concentrated under reduced pressure to give tert-butyl((8-(3′-((2-chloro-5- ((5-cyanopyridin-3-yl)methoxy)-4-(((2-hydroxyethyl)amino)methyl)phenoxy)methyl)-2′-methyl-[1,1′-biphenyl]-3-yl)-4-oxo-4H-pyrido[1,2-a]pyrimidin-3-yl)methyl)(2-hydroxyethyl)carbamate (800 mg, 68% purity, m/z: 831 [M+H]⁺, 416 [M/2+H]⁺ observed) as a brown solid which was used in the next step directly.

### tert-Butyl N-[[8-[3-[3-[[4-[[tert-butoxycarbonyl(2-hydroxyethyl)amino]methyl]-2-chloro-5-[(5-cyano-3-pyridyl)methoxy]phenoxy]methyl]-2-methyl-phenyl]phenyl]-4-oxo-pyrido[1,2-a]pyrimidin-3-yl]methyl]-N-(2-hydroxyethyl)carbamate

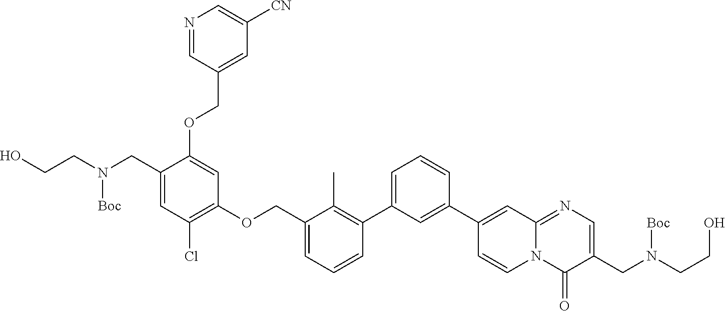 [SMILES: Cc1c(COc2cc(OCc3cncc(C#N)c3)c(CN(CCO)C(=O)OC(C)(C)C)cc2Cl)cccc1-c1cccc(-c2ccn3c(=O)c(CN(CCO)C(=O)OC(C)(C)C)cnc3c2)c1]

To a solution of tert-butyl ((8-(3′-((2-chloro-5-((5-cyanopyridin-3-yl)methoxy)-4- (((2-hydroxyethyl)amino)methyl)phenoxy)methyl)-2′-methyl-[1,1′-biphenyl]-3-yl)-4-oxo-4H-pyrido[1,2-a]pyrimidin-3-yl)methyl)(2-hydroxyethyl)carbamate (800 mg, 0.96 mmol) and di-tert-butyl dicarbonate (0.5 mL, 2.2 mmol) in THE (20 mL) and methanol (10 mL) was added TEA (0.5 mL, 3.6 mmol). The mixture was stirred at r.t. for 5 hrs. The mixture was diluted with THE (30 mL) and washed with half saturated aqueous brine solution (2×20 mL), saturated aqueous brine solution (20 mL), dried over anhydrous sodium sulfate, filtered and concentrated under reduced pressure. The residue was purified by normal phase flash SiO₂ chromatography (ethyl acetate/THF/petroleum ether=1/1/18˜ 2/2/1) to afford tert-butyl N-[[8-[3-[3-[[4-[[tert-butoxycarbonyl(2-hydroxyethyl)amino]methyl]-2-chloro-5-[(5-cyano-3-pyridyl)methoxy]phenoxy]methyl]-2-methyl-phenyl]phenyl]-4-oxo-pyrido[1,2-a]pyrimidin-3-yl]methyl]-N-(2-hydroxyethyl)carbamate (650 mg, 56% yield, m/z: 931 [M+H] observed) as a yellow solid.

### 5-((4-Chloro-2-(((2-hydroxyethyl)amino)methyl)-5-((3′-(3-(((2-hydroxyethyl)amino)methyl)-4-oxo-4H-pyrido[1,2-a]pyrimidin-8-yl)-2-methyl-[1,1′-biphenyl]-3-yl)methoxy)phenoxy)methyl)nicotinonitrile

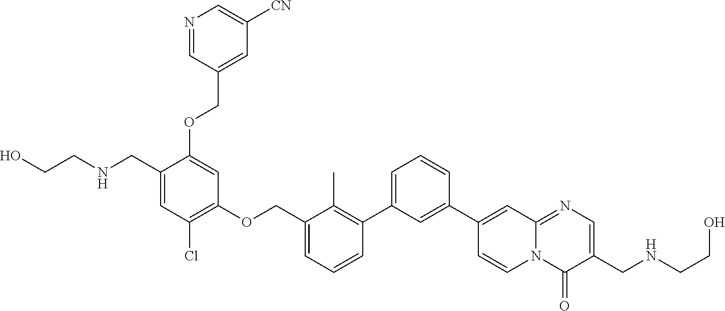 [SMILES: Cc1c(COc2cc(OCc3cncc(C#N)c3)c(CNCCO)cc2Cl)cccc1-c1cccc(-c2ccn3c(=O)c(CNCCO)cnc3c2)c1]

To a solution of tert-butyl N-[[8-[3-[3-[[4-[[tert-butoxycarbonyl(2-hydroxyethyl) amino]methyl]-2-chloro-5-[(5-cyano-3-pyridyl)methoxy]phenoxy]methyl]-2-methyl-phenyl]phenyl]-4-oxo-pyrido[1,2-a]pyrimidin-3-yl]methyl]-N-(2-hydroxyethyl)carbamate (650 mg, 0.70 mmol) in THE (5 mL) at 0° C. was added HCl (4M solution in EtOAc, 5.00 mL, 20 mmol) dropwise. The mixture was stirred at 0° C. for 2 hrs then at r.t. for 12 hrs. The mixture was concentrated and the residue was neutralized with saturated aqueous sodium bicarbonate solution and extracted with THF (100 mL). The organic phase was washed with saturated aqueous brine solution (2×5 mL), dried over anhydrous sodium sulfate, filtered and concentrated under reduced pressure. The residue was triturated with THF (8 mL) at r.t. for 15 min. The resulting yellow solid was collected by filtration to give the semi-purified product (100 mg) as a yellow solid. The solid was purified by reverse phase HPLC to afford 5-((4-chloro-2-(((2-hydroxyethyl)amino)methyl)-5-((3′-(3-(((2-hydroxyethyl)amino)methyl)-4-oxo-4H-pyrido[1,2-a]pyrimidin-8-yl)-2-methyl-[1,1′-biphenyl]-3-yl)methoxy)phenoxy)methyl)nicotinonitrile (46.8 mg, 9% yield, m/z: 731 [M+H]⁺, 753 [M+Na]⁺, 367 [M/2+H]⁺ observed). ¹H NMR (400 MHz, DMSO-d₆): δ 9.04-8.96 (m, 3H), 8.43 (s, 1H), 8.38 (s, 1H), 8.06 (d, J=1.6 Hz, 1H), 7.99 (br d, J=7.6 Hz, 1H), 7.90 (s, 1H), 7.83 (dd, J=8.0, 2.0 Hz, 1H), 7.67 (t, J=8.0 Hz, 1H), 7.57-7.48 (m, 2H), 7.38 (s, 1H), 7.37-7.32 (m, 2H), 7.11 (s, 1H), 5.33 (s, 2H), 5.28 (s, 2H), 4.56-4.35 (m, 2H), 3.71 (s, 2H), 3.66 (s, 2H), 3.49-3.44 (m, 4H), 2.60 (t, J=5.6 Hz, 2H), 2.56-2.53 (m, 2H), 2.30 (s, 3H), 2.23-1.96 (m, 2H).

### Example 5: 5-((4-Chloro-2-(((2-hydroxyethyl)amino)methyl)-5-((3′-(7-(((2-hydroxyethyl)amino)methyl)-4-oxoquinazolin-3(4H)-yl)-2-methyl-[1,1′-biphenyl]-3-yl)methoxy)phenoxy)methyl)nicotinonitrile

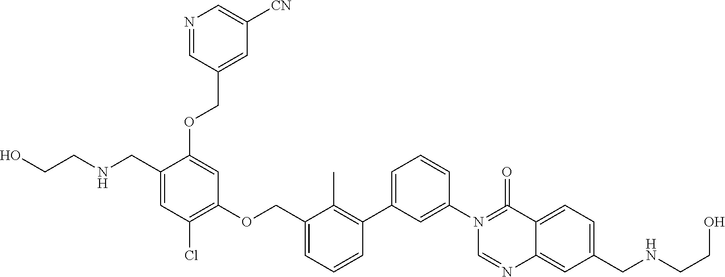

### 2,4-Dioxo-2,4-dihydro-1H-benzo[d][1,3]oxazine-7-carboxylic acid

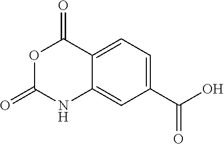 [SMILES: O=C(O)c1ccc2c(=O)oc(=O)[nH]c2c1]

To a solution of 2-aminoterephthalic acid (50 g, 276 mmol) in THE (500 mL) was added bis(trichloromethyl) carbonate (49.2 g, 165 mmol). The mixture was stirred at 50° C. for 12 hr. The mixture was quenched by water (400 mL) and extracted with EtOAc (3×300 mL). The organic layer was dried over anhydrous sodium sulfate, filtered and concentrated to give 2,4-dioxo-2,4-dihydro-1H-benzo[d][1,3]oxazine-7-carboxylic acid (60 g, crude, m/z: 208 [M+H]⁺ observed) as a brown solid.

### 3-Amino-4-((3-bromophenyl)carbamoyl)benzoic acid

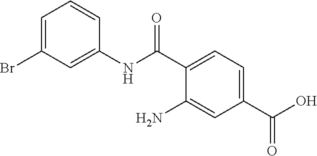 [SMILES: Nc1cc(C(=O)O)ccc1C(=O)Nc1cccc(Br)c1]

To a solution of 2,4-dioxo-2,4-dihydro-1H-benzo[d][1,3]oxazine-7-carboxylic acid (25 g, 120 15 mmol) and 3-bromoaniline (13 mL, 120 mmol) in DMF (500 mL) was added N,N-dimethylpyridin-4-amine (2.95 g, 24 mmol). The mixture was stirred at 80° C. for 18 hr. To the reaction mixture was poured into water (600 mL) and extracted with EtOAc (2×500 mL). The organic layer was washed with water (3×300 mL) and brine (300 mL), dried over anhydrous sodium sulfate, filtered and concentrated. The residue was purified by normal phase SiO₂ chromatography (0-50% EtOAc/petroleum ether) to afford 3-amino-4-((3-bromophenyl)carbamoyl)benzoic acid (17 g, 42% yield) as a yellow solid. ¹H NMR (400 MHz, DMSO-d₆): δ 10.33 (s, 1H), 8.12 (s, 1H), 7.75 (d, J=8.4 Hz, 2H), 7.46 (s, 1H), 7.37-7.35 (m, 2H), 7.17 (d, J=8 Hz, 1H), 6.53 (br s, 2H).

### 3-(3-Bromophenyl)-4-oxo-3,4-dihydroquinazoline-7-carboxylic acid

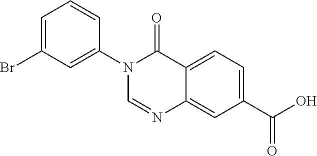 [SMILES: O=C(O)c1ccc2c(=O)n(-c3cccc(Br)c3)cnc2c1]

To a solution of 3-amino-4-((3-bromophenyl)carbamoyl)benzoic acid (25 g, 74.6 mmol) in DMA (250 mL) was added 4-methylbenzenesulfonic acid hydrate (71 g, 373 mmol) and 2-tert-butylperoxy-2-methyl-propane (68.7 mL, 373 mmol). The mixture was stirred at 120° C. for 16 hr. The mixture was poured into water (200 mL) and extracted with EtOAc (3×100 mL). The organic layer was washed with water (3×100 mL) and brine (100 mL), dried over anhydrous sodium sulfate, filtered and concentrated. The residue was washed with a solution of EtOAc/petroleum ether (100 mL, 1:1). The solid was collected and dried to dryness to afford 3-(3-bromophenyl)-4-oxo-quinazoline-7-carboxylic acid (12 g, 45% yield) as a yellow solid. ¹H NMR (400 MHz, DMSO-d₆): δ 8.44 (s, 1H), 8.30 (d, J=8 Hz, 1H), 8.21 (d, J=1.2 Hz, 1H), 8.08 (dd, J=1.6 Hz, J=8.4 Hz, 1H), 7.89 (t, J=2 Hz, 1H), 7.75 (d, J=8 Hz, 1H), 7.61 (d, J=8.4 Hz, 1H), 7.55 (t, J=7.6 Hz, 1H).

### 3-(3-Bromophenyl)-N-methoxy-N-methyl-4-oxo-3,4-dihydroquinazoline-7-carboxamide

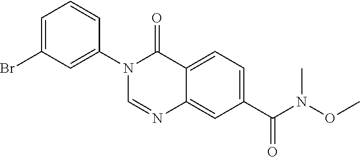 [SMILES: CON(C)C(=O)c1ccc2c(=O)n(-c3cccc(Br)c3)cnc2c1]

To a solution of 3-(3-bromophenyl)-4-oxo-3,4-dihydroquinazoline-7-carboxylic acid (10 g, 28.9 mmol) and N,O-dimethylhydroxylamine hydrochloride (4.24 g, 43.5 mmol) in DMF (100 mL) was added 2-(3H-[1,2,3]triazolo[4,5-b]pyridin-3-yl)-1,1,3,3-tetramethyluronium hexafluorophosphate (14.3 g, 37.6 mmol) and N-ethyl-N-isopropylpropan-2-amine (15 mL, 86.9 mmol). The mixture was stirred at r.t. for 12 hr. Water (150 mL) was added and the mixture was extracted with ethyl acetate (2×150 mL). The combined organic phase was dried with anhydrous sodium sulfate, filtered and concentrated. The residue was purified by normal phase SiO₂ chromatography (0-80% EtOAc/petroleum ether) to afford 3-(3-bromophenyl)-N-methoxy-N-methyl-4-oxo-quinazoline-7-carboxamide (9 g, 80% yield) as a yellow solid. ¹H NMR (400 MHz, DMSO-d₆): δ 8.42 (s, 1H), 8.25 (d, J=8 Hz, 1H), 7.88 (d, J=4.4 Hz, 2H), 7.73 (t, J=6.8 Hz, 2H), 7.61 (d, J=8 Hz, 1H), 7.55 (t, J=8 Hz, 1H), 3.56 (s, 3H), 3.32 (s, 3H).

### 3-(3-Bromophenyl)-4-oxo-3,4-dihydroquinazoline-7-carbaldehyde

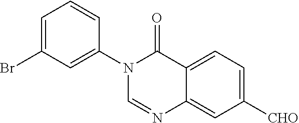 [SMILES: O=Cc1ccc2c(=O)n(-c3cccc(Br)c3)cnc2c1]

To a solution of 3-(3-bromophenyl)-N-methoxy-N-methyl-4-oxo-3,4-dihydroquinazoline-7-carboxamide (4 g, 10.3 mmol) in THE (40 mL) was added lithium aluminium hydride (0.78 5 g, 20.6 mmol). The mixture was stirred at −60° C. for 1 hr. The mixture was quenched with saturated ammonium chloride (50 mL) and extracted with EtOAc (2×50 mL). The organic layer was dried over anhydrous sodium sulfate, filtered and concentrated. The residue was suspended in EtOAc/petroleum ether (1:1, 20 mL) and stirred for 0.5 h. The mixture was filtered and the yellow solid was collected and dried to dryness to afford 3-(3-bromophenyl)-4-oxo-3,4-dihydroquinazoline-7-carbaldehyde (2 g, 48% yield) as a yellow solid. ¹H NMR (400 MHz, DMSO-d₆): δ 10.24 (s, 1H), 8.49 (s, 1H), 8.37 (d, J=8 Hz, 1H), 8.29 (d, J=1.2 Hz, 1H), 8.03 (dd, J=1.2 Hz, J=8 Hz, 1 Hz), 7.91 (t, J=2 Hz, 1H), 7.77-7.75 (m, 1H), 7.62-7.61 (m, 1H), 7.58-7.54 (m, 1H).

### 5-((4-Chloro-2-formyl-5-((3′-(7-formyl-4-oxoquinazolin-3(4H)-yl)-2-methyl-[1,1′-biphenyl]-3-yl)methoxy)phenoxy)methyl)nicotinonitrile

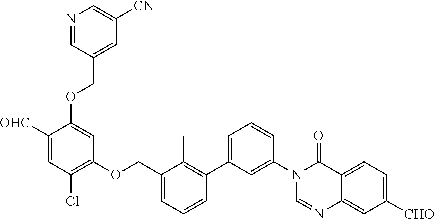 [SMILES: Cc1c(COc2cc(OCc3cncc(C#N)c3)c(C=O)cc2Cl)cccc1-c1cccc(-n2cnc3cc(C=O)ccc3c2=O)c1]

To a solution of 3-(3-bromophenyl)-4-oxo-3,4-dihydroquinazoline-7-carbaldehyde (300 mg, 911 umol) and 5-((4-chloro-2-formyl-5-((2-methyl-3-(3,3,4,4-tetramethylborolan-1-yl)benzyl)oxy)phenoxy)methyl)nicotinonitrile (0.47 g, 0.9 mmol) in dioxane (1 mL) and H₂O (0.2 mL) was added cesium carbonate (594 mg, 0.18 mmol) and cyclopenta-2,4-dien-1-yl(diphenyl)phosphane;dichloropalladium;iron(2+) (66.7 mg, 91 umol). The mixture was stirred at 80° C. for 1 hr. To the mixture was added water (5 mL). The mixture was extracted with ethyl acetate (2×5 mL). The combined organic phase was dried with anhydrous sodium sulfate, filtered and concentrated. The residue was purified by normal phase SiO₂ chromatography (0-80% EtOAc/petroleum ether) to afford 5-((4-chloro-2-formyl-5-((3′-(7-formyl-4-oxoquinazolin-3(4H)-yl)-2-methyl-[1,1′-biphenyl]-3-yl)methoxy)phenoxy)methyl)nicotinonitrile (300 mg, 51% yield) as a yellow solid. ¹H NMR (400 MHz, CDCl₃): δ 10.21 (s, 1H), 10.15 (s, 1H), 8.83 (d, J=2 Hz, 3H), 8.44 (d, J=8 Hz, 1H), 8.19 (d, J=11.2 Hz, 2H), 8.02-7.99 (m, 3H), 7.86 (s, 1H), 7.58 (t, J=7.6 Hz, 1H), 7.41-7.37 (m, 5H), 7.27-7.25 (m, 2H), 6.59-6.58 (m, 1H), 5.18-5.16 (m, 5H), 2.27 (s, 3H).

### 5-((4-Chloro-2-(((2-hydroxyethyl)amino)methyl)-5-((3′-(7-(((2-hydroxyethyl)amino)methyl)-4-oxoquinazolin-3(4H)-yl)-2-methyl-[1,1′-biphenyl]-3-yl)methoxy)phenoxy)methyl)nicotinonitrile

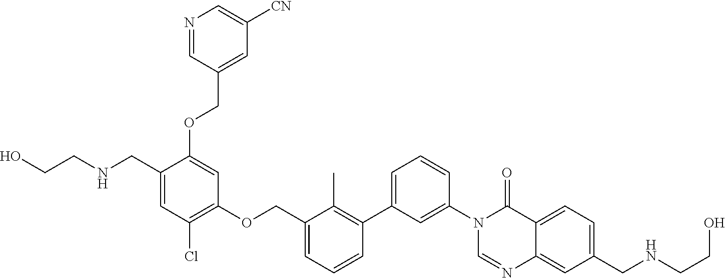 [SMILES: Cc1c(COc2cc(OCc3cncc(C#N)c3)c(CNCCO)cc2Cl)cccc1-c1cccc(-n2cnc3cc(CNCCO)ccc3c2=O)c1]

To a solution of 5-((4-chloro-2-formyl-5-((3′-(7-formyl-4-oxoquinazolin-3(4H)-yl)-2-methyl-[1,1′-biphenyl]-3-yl)methoxy)phenoxy)methyl)nicotinonitrile (280 mg, 0.44 mmol) and 2-aminoethanol (79.2 uL, 0.13 mmol) in MeOH (1 mL) and THE (5 mL) was added acetic acid (24.9 uL, 0.44 mmol) The mixture was stirred at r.t. for 12 hr. Sodium cyanoborohydride (55 mg, 0.87 mmol) was added and the mixture was stirred at r.t. for 1 hr. The reaction mixture was concentrated. The residue was purified by reverse phase HPLC to afford 5-((4-chloro-2-(((2-hydroxyethyl)amino)methyl)-5-((3′-(7-(((2-hydroxyethyl)amino)methyl)-4-oxoquinazolin-3(4H)-yl)-2-methyl-[1,1′-biphenyl]-3-yl)methoxy)phenoxy)methyl)nicotinonitrile (70.95 mg, 21.68% yield, m/z: 731 [M+H]⁺ observed) as a white solid. ¹H NMR (400 MHz, CD₃OD): δ8.88 (dd, J=2 Hz, J=16 Hz, 2H), 8.37 (s, 1H), 8.31-8.26 (m, 2H), 7.75 (s, 1H), 7.66-7.61 (m, 2H), 7.51-7.46 (m, 4H), 7.33 (s, 1H), 7.31-7.24 (m, 2H), 6.92 (s, 1H), 5.28 (s, 2H), 5.25 (s, 2H), 3.99 (s, 2H), 3.75 (s, 2H), 3.69 (t, J=5.2 Hz, 2H), 3.63 (t, J=5.6 Hz, 2H), 2.76 (t, J=5.6 Hz, 2H), 2.68 (t, J=5.6 Hz, 2H), 2.34 (s, 3H).

### Example 6: 5-((4-Chloro-2-(((2-hydroxyethyl)amino)methyl)-5-((3′-(7-(((2-hydroxyethyl)amino)methyl)-4-oxoquinazolin-3(4H)-yl)-2,2′-dimethyl-[1,1′-biphenyl]-3-yl)methoxy)phenoxy)methyl)nicotinonitrile

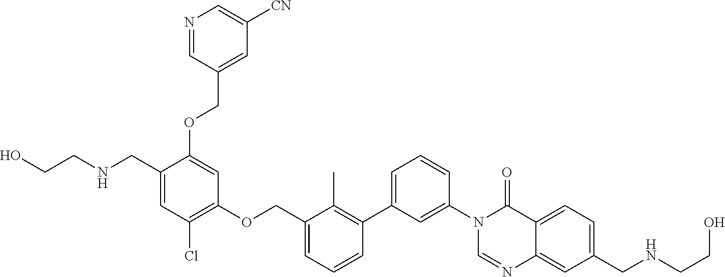 [SMILES: Cc1c(COc2cc(OCc3cncc(C#N)c3)c(CNCCO)cc2Cl)cccc1-c1cccc(-n2cnc3cc(CNCCO)ccc3c2=O)c1]

### 3-Amino-4-((3-bromo-2-methylphenyl)carbamoyl)benzoic acid

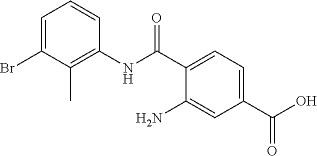 [SMILES: Cc1c(Br)cccc1NC(=O)c1ccc(C(=O)O)cc1N]

To a solution of 2,4-dioxo-2,4-dihydro-1H-benzo[d][1,3]oxazine-7-carboxylic acid (25 g, 121 mmol) and 3-bromo-2-methylaniline (14.9 mL, 121 mmol) in DMF (250 mL) was added N,N-dimethylpyridin-4-amine (2.95 g, 24.14 mmol). The mixture was stirred at 80° C. for 18 hr. To the reaction mixture was poured into water (600 mL) and extracted with EtOAc (2×500 mL). The organic layer was washed with water (3×300 mL) and brine (300 mL), dried over anhydrous sodium sulfate, filtered and concentrated. The residue was purified by normal phase SiO₂ chromatography (0-50% EtOAc/petroleum ether) to afford 3-amino-4-((3-bromo-2-methylphenyl)carbamoyl)benzoic acid (10 g, 16.3% yield, m/z: 349 [M]⁺, 351[M+2]⁺ observed) as a yellow solid.

### 3-(3-Bromo-2-methylphenyl)-4-oxo-3,4-dihydroquinazoline-7-carboxylic acid

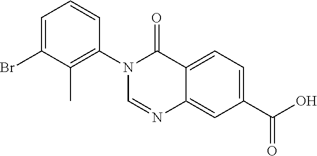 [SMILES: Cc1c(Br)cccc1-n1cnc2cc(C(=O)O)ccc2c1=O]

To a solution of 3-amino-4-((3-bromo-2-methylphenyl)carbamoyl)benzoic acid (25 g, 71.6 mmol) in DMA (250 mL) was added 4-methylbenzenesulfonic acid hydrate (68 g, 358 mmol) and 2-tert-butylperoxy-2-methyl-propane (65.9 mL, 358 mmol). The mixture was stirred at 120° C. for 16 hr. The mixture was poured into water (200 mL) and extracted with EtOAc (3×100 mL). The organic layer was washed with water (3×100 mL) and brine (100 mL), dried over anhydrous sodium sulfate, filtered and concentrated. The residue was washed with a solution of EtOAc/petroleum ether (100 mL, 1:1) and the resulting yellow solid was collected to afford 3-(3-bromo-2-methyl-phenyl)-4-oxo-quinazoline-7-carboxylic acid (5 g, 20% yield) as a yellow solid. ¹H NMR (400 MHz, DMSO-d₆): δ 8.39 (s, 1H), 8.31 (d, J=8.4 Hz, 1H), 8.24 (d, J=1.6 Hz, 1H), 8.09 (dd, J=1.6 Hz, J=8.4 Hz, 1H), 7.82 (dd, J=7.6 Hz, J=0.8 Hz, 1H), 7.52 (dd, J=0.8 Hz, J=8 Hz, 1H), 7.37 (t, J=8 Hz, 1H), 2.16 (s, 3H).

### 3-(3-Bromo-2-methylphenyl)-N-methoxy-N-methyl-4-oxo-3,4-dihydroquinazoline-7-carboxamide

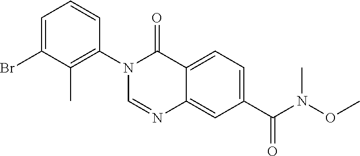 [SMILES: CON(C)C(=O)c1ccc2c(=O)n(-c3cccc(Br)c3C)cnc2c1]

To a solution of 3-(3-bromo-2-methylphenyl)-4-oxo-3,4-dihydroquinazoline-7-carboxylic acid (5 g, 13.9 mmol) and N-methoxymethanamine;hydrochloride (2.04 g, 20.8 mmol) in DMF (50 mL) was added 2-(3H-[1,2,3]triazolo[4,5-b]pyridin-3-yl)-1,1,3,3-tetramethyluronium hexafluorophosphate (6.88 g, 18.1 mmol) and N-ethyl-N-isopropylpropan-2-amine (7.27 mL, 41.7 mmol). The mixture was stirred at r.t. for 12 hr. The combined organic layers were dried over anhydrous sodium sulfate, filtered and concentrated under reduced pressure. The residue was purified by normal phase SiO₂ chromatography (0-80% EtOAc/petroleum ether) to afford 3-(3-bromo-2-methylphenyl)-N-methoxy-N-methyl-4-oxo-3,4-dihydroquinazoline-7-carboxamide (5 g, 89% yield) as a white solid. ¹H NMR (400 MHz, CDCl₃): δ 8.32 (d, J=8.4 Hz, 1H), 8.00 (d, J=1.2 Hz, 1H), 7.75 (s, 2H), 7.74 (dd, J=8.4 Hz, J=1H), 3.25 (s, 3H), 3.36 (s, 3H), 2.19 (s, 3H).

### 3-(3-Bromo-2-methylphenyl)-4-oxo-3,4-dihydroquinazoline-7-carbaldehyde

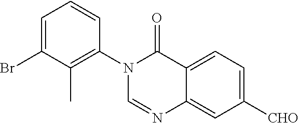 [SMILES: Cc1c(Br)cccc1-n1cnc2cc(C=O)ccc2c1=O]

To a solution of 3-(3-bromo-2-methylphenyl)-N-methoxy-N-methyl-4-oxo-3,4-dihydroquinazoline-7-carboxamide (2 g, 4.97 mmol) in THE (1 mL) was added lithium aluminium hydride (377 mg, 9.94 mmol). The mixture was stirred at −60° C. for 1 hr. The mixture was quenched with saturated ammonium chloride (10 mL) and extracted with EtOAc (2×20 mL). The organic layer was dried over anhydrous sodium sulfate, filtered and concentrated. The residue was suspended in EtOAc/petroleum ether(1:1, 10 mL) and stirred for 0.5 h. The mixture was filtered and the yellow solid was collected and dried to afford 3-(3-bromo-2-methylphenyl)-4-oxo-3,4-dihydroquinazoline-7-carbaldehyde (1.2 g, 59% yield) as a yellow solid. ¹H NMR (400 MHz, DMSO-d₆): δ 10.22 (s, 1H), 8.41 (s, 1H), 8.35 (d, J=8 Hz, 1H), 8.29 (s, 1H), 8.02 (dd, J=3.2 Hz, J=8.4 Hz, 1H), 7.81 (d, J=8 Hz, 1H), 7.53 (d, J=7.6 Hz, 1H), 7.35 (t, J=8 Hz, 1H), 2.14 (s, 3H).

### 5-((4-Chloro-2-formyl-5-((3′-(7-formyl-4-oxoquinazolin-3(4H)-yl)-2,2′-dimethyl-[1,1′-biphenyl]-3-yl)methoxy)phenoxy)methyl)nicotinonitrile

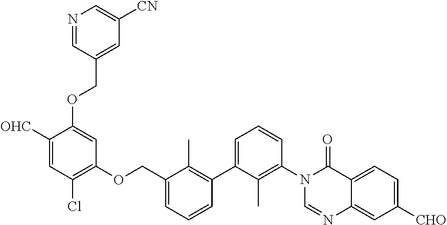 [SMILES: Cc1c(COc2cc(OCc3cncc(C#N)c3)c(C=O)cc2Cl)cccc1-c1cccc(-n2cnc3cc(C=O)ccc3c2=O)c1C]

To a solution of 3-(3-bromo-2-methylphenyl)-4-oxo-3,4-dihydroquinazoline-7-carbaldehyde (600 mg, 1.75 mmol) and 5-((4-chloro-2-formyl-5-((2-methyl-3-(3,3,4,4-tetramethylborolan-1-yl)benzyl)oxy)phenoxy)methyl)nicotinonitrile (907 mg, 1.75 mmol) in dioxane (5 mL) and H₂O (1 mL) was added cesium carbonate (1.14 g, 3.50 mmol) and cyclopenta-2,4-dien-1-yl(diphenyl)phosphane;dichloropalladium;iron(2+) (127 mg, 174 umol). The mixture was stirred at 80° C. for 1 hr. The reaction mixture was filtered. The filtrate was diluted with H₂O 25 mL and extracted with EtOAc (2×20 mL). The combined organic layers were dried over anhydrous sodium sulfate, filtered and concentrated. The residue was purified by normal phase SiO₂ chromatography (0-80% EtOAc/petroleum ether) to afford 5-((4-chloro-2-formyl-5-((3′-(7-formyl-4-oxoquinazolin-3(4H)-yl)-2,2′-dimethyl-[1,1′-biphenyl]-3-yl)methoxy)phenoxy)methyl)nicotinonitrile (1 g, 67% yield) as a yellow solid. ¹H NMR (400 MHz, DMSO-d₆): δ 10.26 (s, 1H), 10.23 (s, 1H), 9.00 (dd, J=6.8 Hz, J=2 Hz, 2H), 8.48-8.40 (m, 3H), 8.29 (s, 1H), 8.05 (d, J=1.2 Hz, 1H), 7.74 (s, 1H), 7.56 (d, J=7.6 Hz, 1H), 7.50 (d, J=8.4 Hz, 2H), 7.38-7.32 (m, 2H), 7.27 (s, 1H), 7.21 (d, J=7.6 Hz, 1H), 5.49-5.46 (m, 4H), 2.15 (d, J=2.4 Hz, 3H), 1.81 (d, J=1.2 Hz, 3H),

### 5-((4-Chloro-2-(((2-hydroxyethyl)amino)methyl)-5-((3′-(7-(((2-hydroxyethyl)amino)methyl)-4-oxoquinazolin-3(4H)-yl)-2,2′-dimethyl-[1,1′-biphenyl]-3-yl)methoxy)phenoxy)methyl)nicotinonitrile

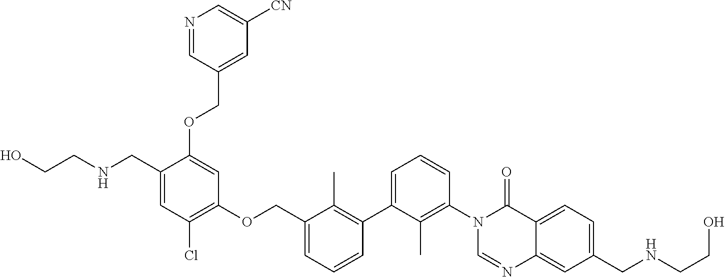 [SMILES: Cc1c(COc2cc(OCc3cncc(C#N)c3)c(CNCCO)cc2Cl)cccc1-c1cccc(-n2cnc3cc(CNCCO)ccc3c2=O)c1C]

To a solution of 5-((4-chloro-2-formyl-5-((3′-(7-formyl-4-oxoquinazolin-3(4H)-yl)-2,2′-dimethyl-[1,1′-biphenyl]-3-yl)methoxy)phenoxy)methyl)nicotinonitrile (500 mg, 0.77 mmol) and 2-aminoethanol (0.18 mL, 0.3 mmol) in MeOH (1 mL) and THE (1 mL) was added acetic acid (0.77 mmol, 43.6 uL) The mixture was stirred at r.t. for 12 hr. Sodium cyanoborohydride (95.9 mg, 0.15 mmol) was added and the mixture was stirred at r.t. for 1 hr. To the mixture was added water (5 mL) and the mixture was extracted with ethyl acetate (2×5 mL). The combined organic phase was dried with anhydrous sodium sulfate, filtered and concentrated to give the residue. The residue was purified by prep-HPLC (column: Phenomenex Gemini-NX 150*30 mm*5 um; mobile phase: [water (10 mM NH₄HCO₃)-ACN]; B %: 35%-50%, 10 min) to give 5-((4-chloro-2-(((2-hydroxyethyl)amino)methyl)-5-((3′-(7-(((2-hydroxyethyl)amino)methyl)-4-oxoquinazolin-3(4H)-yl)-2,2′-dimethyl-[1,1′-biphenyl]-3-yl)methoxy)phenoxy)methyl)nicotinonitrile (183.44 mg, 30.89% yield, m/z: 745 [M+H]⁺ observed) as a white solid. ¹H NMR (400 MHz, CD3OD): δ8.92 (d, J=1.6 Hz, 1H), 8.88 (d, J=1.6 Hz, 1H), 8.34 (d, J=2 Hz, 1H), 8.29 (t, J=8 Hz, 2), 7.78 (s, 1H), 7.64 (dd, J=0.8 Hz, J=9.2 Hz, 1H), 7.51-7.48 (m, 2H), 7.40 (d, J=8 Hz, 1H), 7.34-7.31 (m, 2H), 7.28 (t, J=7.6 Hz, 1H), 7.16-7.14 (m, 1H), 6.97 (s, 1H), 5.29-5.26 (m, 4H), 4.00 (s, 2H), 3.75 (s, 2H), 3.70 (t, J=5.6 Hz, 2H), 3.63 (t, J=5.2 Hz, 2H), 2.77 (t, J=5.6 Hz, 2H), 2.68 (t, J=5.2 Hz, 2H), 2.17 (d, J=12 Hz, 3H), 1.82 (s, 3H).

### Example 7: (S)-2-(((8-(3′-((4-((((S)-2-Carboxy-1-hydroxypropan-2-yl)amino)methyl)-2-chloro-5-((5-cyanopyridin-3-yl)methoxy)phenoxy)methyl)-2,2′-dimethyl-[1,1′-biphenyl]-3-yl)-4-oxo-4H-pyrido[1,2-a]pyrimidin-3-yl)methyl)amino)-3-hydroxy-2-methylpropanoic acid

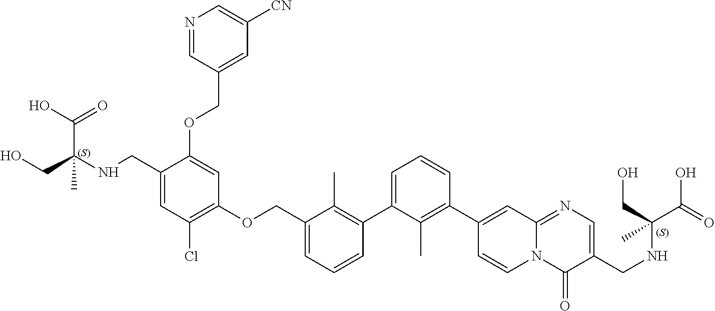

### 5-[[4-Chloro-2-formyl-5-[[3-[3-(3-formyl-4-oxo-pyrido[1,2-a]pyrimidin-8-yl)-2-methyl-phenyl]-2-methyl-phenyl]methoxy]phenoxy]methyl]pyridine-3-carbonitrile

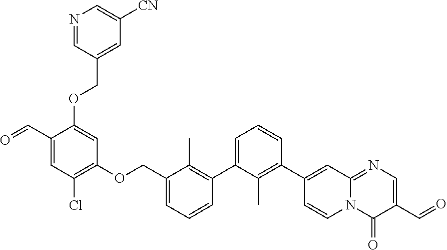 [SMILES: Cc1c(COc2cc(OCc3cncc(C#N)c3)c(C=O)cc2Cl)cccc1-c1cccc(-c2ccn3c(=O)c(C=O)cnc3c2)c1C]

To a mixture of 8-(3-bromo-2-methyl-phenyl)-4-oxo-pyrido[1,2-a]pyrimidine-3-carbaldehyde (0.9 g, 2.62 mmol) and 5-[[4-chloro-2-formyl-5-[[2-methyl-3-(4,4,5,5-tetramethyl −1,3,2-dioxaborolan-2-yl)phenyl]methoxy]phenoxy]methyl]pyridine-3-carbonitrile (2 g, 4 mmol) in dioxane (13.5 mL) and H₂O (4.5 mL) was added cesium carbonate (2.56 g, 7.87 mmol) and Pd(dppf)Cl₂.CH₂Cl₂ (0.2 g, 0.26 mmol). The mixture was stirred at 80° C. for 1 h. The reaction mixture was treated with water (10 mL) and then extracted with EtOAc (2×10 mL) and THF (2×10 mL). The combined organic layers were concentrated under reduced pressure to give a residue. The crude product was triturated with EtOAc (10 mL) at r.t. for 5 min. The resulting black solid was collected by filtration to afford 5-[[4-chloro-2-formyl-5-[[3-[3-(3-formyl-4-oxo-pyrido[1,2-a]pyrimidin-8-yl)-2-methyl-phenyl]-2-methyl-phenyl]methoxy]phenoxy]methyl]pyridine-3-carbonitrile (0.6 g, 34% yield, m/z: 655 [M+H]⁺ observed).

### (S)-2-(((8-(3′-((4-((((S)-2-Carboxy-1-hydroxypropan-2-yl)amino)methyl)-2-chloro-5-((5-cyanopyridin-3-yl)methoxy)phenoxy)methyl)-2,2′-dimethyl-[1,1′-biphenyl]-3-yl)-4-oxo-4H-pyrido[1,2-a]pyrimidin-3-yl)methyl)amino)-3-hydroxy-2-methylpropanoic acid

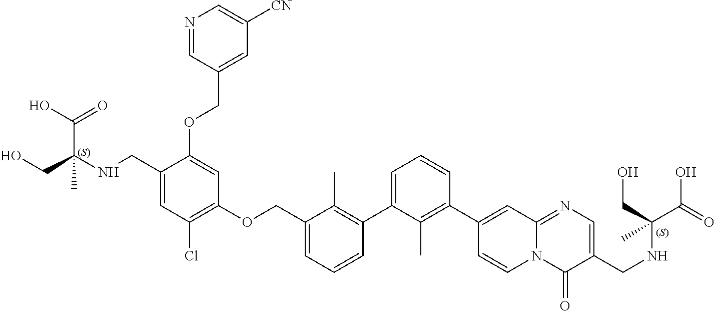

To a mixture of 5-[[4-chloro-2-formyl-5-[[3-[3-(3-formyl-4-oxo-pyrido[1,2-a]pyrimidin-8-yl)-2-methyl-phenyl]-2-methyl-phenyl]methoxy]phenoxy]methyl]pyridine-3-carbonitrile (0.3 g, 0.46 mmol) in DCM (2.5 mL) was added a solution of (S)-2-amino-3-hydroxy-2-methylpropanoic acid (1 g, 9 mmol) and KOH (0.5 g, 9 mmol) in EtOH (12.5 mL). The mixture was stirred at r.t. for 1 h. Glacial HOAc (0.5 mL, 9 mmol) and sodium triacetoxyborohydride (2 g, 9 mmol) was added to the mixture and sonicated for 1 min and stirred at r.t. for 0.5 h. The reaction mixture was filtered and the filtrate was purified by reverse phase HPLC to afford (S)-2-(((8-(3′-((4-((((S)-2-carboxy-1-hydroxypropan-2-yl)amino)methyl)-2-chloro-5-((5-cyanopyridin-3-yl)methoxy)phenoxy)methyl)-2,2′-dimethyl-[1,1′-biphenyl]-3-yl)-4-oxo-4H-pyrido[1,2-a]pyrimidin-3-yl)methyl)amino)-3-hydroxy-2-methylpropanoic acid as a white solid (19.46 mg). m/z: 861 [M+H]⁺ observed. ¹H NMR (400 MHz, CD₃OD): δ. 9.18 (s, 1H), 9.00 (s, 1H), 8.92 (s, 1H), 8.57 (s, 1H), 8.45 (s, 1H), 7.75 (s, 1H), 7.45 (m, 5H), 7.32 (s, 2H), 7.19 (d, J=6.8 Hz, 1H), 7.09 (s, 1H), (t, J=8 Hz, 2H), 5.35 (d, J=24 Hz, 4H), 4.25 (m, 3H), 4.03 (d, J=12 Hz, 1H), 3.93 (d, J=12 Hz, 1H), 3.82 (d, J=12.8 Hz, 1H), 3.72 (d, J=12 Hz, 1H), 2.18 (s, 3H), 2.04 (s, 3H), 1.58 (s, 3H), 1.46 (s, 3H).

### Example 8: 5-((4-Chloro-5-((3′-(1-(2-((2-hydroxyethyl)amino)ethyl)-4-methyl-2,5-dioxo-2,3,4,5-tetrahydro-1H-benzo[e][1,4]diazepin-7-yl)-2,2′-dimethyl-[1,1′-biphenyl]-3-yl)methoxy)-2-(((2-hydroxyethyl)amino)methyl)phenoxy)methyl)nicotinonitrile

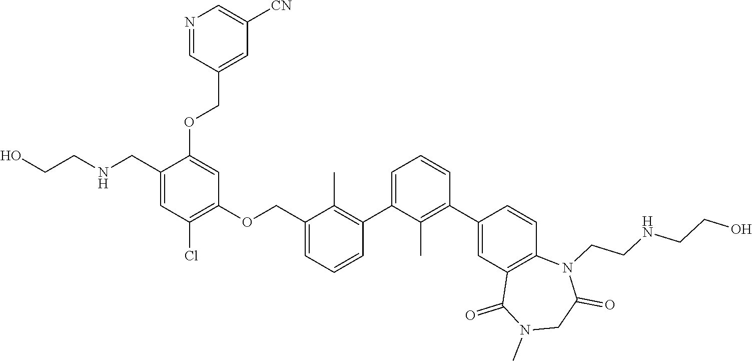 [SMILES: Cc1c(COc2cc(OCc3cncc(C#N)c3)c(CNCCO)cc2Cl)cccc1-c1cccc(-c2ccc3c(c2)C(=O)N(C)CC(=O)N3CCNCCO)c1C]

### 7-(3-Bromo-2-methylphenyl)-4-methyl-3,4-dihydro-1H-benzo[e][1,4]diazepine-2,5-dione:

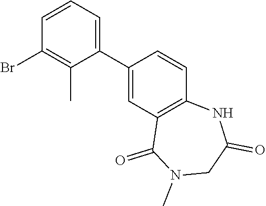 [SMILES: Cc1c(Br)cccc1-c1ccc2c(c1)C(=O)N(C)CC(=O)N2]

To a mixture of 7-bromo-4-methyl-3,4-dihydro-1H-benzo[e][1,4]diazepine-2,5-dione (14 g, 52 mmol) and (3-bromo-2-methylphenyl)boronic acid (12.6 g, 58 mmol) in THF/DMF/H₂O (180 mL/180 mL/105 mL) was added Pd(PPh₃)₄ (4.8 g, 4 mmol) and sodium carbonate (6.6 g, 62.5 mmol) in one portion under N₂. The mixture was stirred at 80° C. for 16 hours. The combined two batches of the mixture were concentrated directly. The residue was triturated with water (300 mL) for 10 min and filtered. The filter cake was washed with water (200 mL) and dried in vacuo. The resultant solid was triturated again with EtOAc (200 mL) for 10 min and filtered. The filter cake was washed with EtOAc (100 mL) and dried in vacuo to give 7-(3-bromo-2-methylphenyl)-4-methyl-3,4-dihydro-1H-benzo[e][1,4]diazepine-2,5-dione as white solid (30 g, 80% yield). ¹H NMR (400 MHz, DMSO-d₆): δ 10.58 (s, 1H), 7.64 (m, 2H), 7.49 (m, 1H), 7.20 (m, 3H), 3.92 (s, 2H), 3.13 (s, 3H), 2.28 (s, 3H).

### 7-(3-Bromo-2-methylphenyl)-1-(2,2-dimethoxyethyl)-4-methyl-3,4-dihydro-1H-benzo[e][1,4]diazepine-2,5-dione

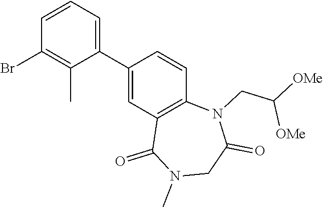 [SMILES: COC(CN1C(=O)CN(C)C(=O)c2cc(-c3cccc(Br)c3C)ccc21)OC]

To a mixture of 7-(3-bromo-2-methylphenyl)-4-methyl-3,4-dihydro-1H-benzo[e][1,4]diazepine-2,5-dione (4 g, 11 mmol) and 2-bromo-1,1-dimethoxy-ethane (2.8 g, 16.7 mmol) in DMF (140 mL) was added potassium carbonate (9 g, 28 mmol) and potassium iodide (0.18 g, 1 mmol). The mixture was stirred at 60° C. for 48 hours. The 7 batches of the mixture were combined and filtered. The filtrate was concentrated to 150 mL and purified by reverse phase HPLC to afford 7-(3-bromo-2-methylphenyl)-1-(2,2-dimethoxyethyl)-4-methyl-3,4-dihydro-1H-benzo[e][1,4]diazepine-2,5-dione as yellow solid (17 g, 50% yield). ¹H NMR (400 MHz, CDCl₃): δ 7.70 (d, J=2 Hz, 1H), 7.49 (m, 2H), 7.36 (m, 1H), 7.10 (m, 1H), 7.03 (m, 1H), 4.62 (m, 1H), 4.07 (m, 2H), 3.79 (m, 1H), 3.69 (m, 1H), 3.44 (s, 3H), 3.28 (s, 3H), 3.22 (s, 3H), 2.26 (s, 3H).

### 5-((4-Chloro-5-((3′-(1-(2,2-dimethoxyethyl)-4-methyl-2,5-dioxo-2,3,4,5-tetrahydro-1H-benzo[e][1,4]diazepin-7-yl)-2,2′-dimethyl-[1,1′-biphenyl]-3-yl)methoxy)-2-formylphenoxy)methyl)nicotinonitrile

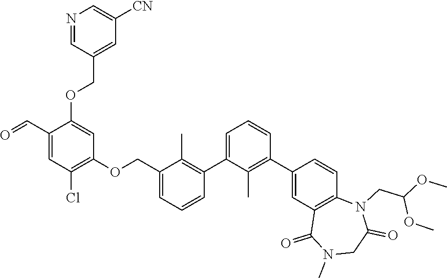 [SMILES: COC(CN1C(=O)CN(C)C(=O)c2cc(-c3cccc(-c4cccc(COc5cc(OCc6cncc(C#N)c6)c(C=O)cc5Cl)c4C)c3C)ccc21)OC]

A mixture of 7-(3-bromo-2-methylphenyl)-1-(2,2-dimethoxyethyl)-4-methyl-3,4-dihydro-1H-benzo[e][1,4]diazepine-2,5-dione (3 g, 6.7 mmol), 5-((4-chloro-2-formyl-5-((2-methyl-3-(4,4,5,5-tetramethyl-1,3,2-dioxaborolan-2-yl)benzyl)oxy)phenoxy)methyl)nicotinonitrile (4.5 g, 8.7 mmol) and cesium carbonate (3.3 g, 10 mmol) in dioxane/H₂O (3/1, 200 mL) was degassed and purged with N₂ for 3 times, and then the mixture was added Pd(dppf)Cl₂ (0.5 g, 0.67 mmol). After that the mixture was stirred at 80° C. for 1 hr. The reaction mixture was treated with water (100 mL), and then extracted with EtOAc 200 mL (2×100 mL). The combined organic layers were washed with brine 100 mL, dried over anhydrous sodium sulfate, filtered and concentrated under reduced pressure to give a residue. The residue was purified by normal phase SiO₂ chromatography (15%-100% EtOAc/petroleum ether) to afford 5-((4-chloro-5-((3′-(1-(2,2-dimethoxyethyl)-4-methyl-2,5-dioxo-2,3,4,5-tetrahydro-1H-benzo[e][1,4]diazepin-7-yl)-2,2′-dimethyl-[1,1′-biphenyl]-3-yl)methoxy)-2-formylphenoxy)methyl)nicotinonitrile as a yellow solid (3 g, 59% yield). ¹H NMR (400 MHz, CDCl₃): δ 10.30 (s, 1H), 8.93 (s, 2H), 8.12 (s, 1H), 7.95 (m, 1H), 7.87 (s, 1H), 7.52 (m, 3H), 7.32 (m, 2H), 7.25 (m, 2H), 7.16 (m, 1H), 6.69 (s, 1H), 5.27 (m, 4H), 4.71 (t, J=5.2 Hz, 1H), 4.17 (m, 2H), 3.85 (m, 1H), 3.70 (m, 1H), 3.43 (s, 3H), 3.36 (m, 3H), 3.31 (s, 3H), 2.15 (m, 3H), 1.95 (m, 3H).

### 5-((4-Chloro-5-((2,2′-dimethyl-3′-(4-methyl-2,5-dioxo-1-(2-oxoethyl)-2,3,4,5-tetrahydro-1H-benzo[e][1,4]diazepin-7-yl)-[1,1′-biphenyl]-3-yl)methoxy)-2-formylphenoxy)methyl)nicotinonitrile

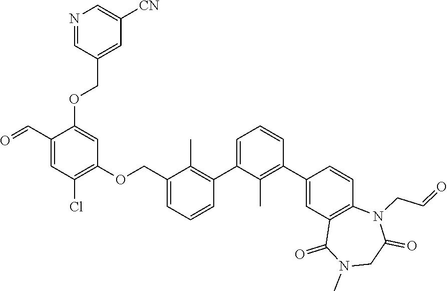 [SMILES: Cc1c(COc2cc(OCc3cncc(C#N)c3)c(C=O)cc2Cl)cccc1-c1cccc(-c2ccc3c(c2)C(=O)N(C)CC(=O)N3CC=O)c1C]

A mixture of 5-((4-chloro-5-((3′-(1-(2,2-dimethoxyethyl)-4-methyl-2,5-dioxo-2,3,4,5-tetrahydro-1H-benzo[e][1,4]diazepin-7-yl)-2,2′-dimethyl-[1,1′-biphenyl]-3-yl)methoxy)-2-formylphenoxy)methyl)nicotinonitrile (0.5 g, 0.66 mmol) in 4 N HCl solution in EtOAc (70 mL) was stirred at r.t. for 2 h. The reaction mixture was concentrated under reduced pressure to give 5-((4-chloro-5-((2,2′-dimethyl-3′-(4-methyl-2,5-dioxo-1-(2-oxoethyl)-2,3,4,5-tetrahydro-1H-benzo[e][1,4]diazepin-7-yl)-[1,1′-biphenyl]-3-yl)methoxy)-2-formylphenoxy)methyl)nicotinonitrile as a yellow solid (0.5 g, crude) which was used to next step directly.

### tert-Butyl N-[[4-[[3-[3-[1-[2-[tert-butoxycarbonyl(2-hydroxyethyl)amino]ethyl]-4-methyl-2,5-dioxo-3H-1,4-benzodiazepin-7-yl]-2-methyl-phenyl]-2-methyl-phenyl]methoxy]-5-chloro-2-[(5-cyano-3-pyridyl)methoxy]phenyl]methyl]-N-(2-hydroxyethyl)carbamate

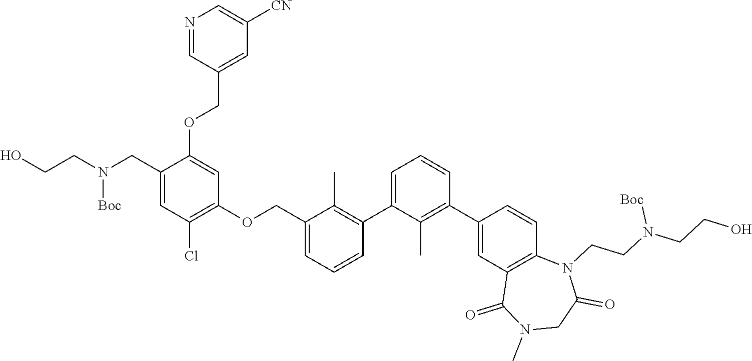 [SMILES: Cc1c(COc2cc(OCc3cncc(C#N)c3)c(CN(CCO)C(=O)OC(C)(C)C)cc2Cl)cccc1-c1cccc(-c2ccc3c(c2)C(=O)N(C)CC(=O)N3CCN(CCO)C(=O)OC(C)(C)C)c1C]

A mixture of 5-((4-chloro-5-((2,2′-dimethyl-3′-(4-methyl-2,5-dioxo-1-(2-oxoethyl)-2,3,4,5-tetrahydro-1H-benzo[e][1,4]diazepin-7-yl)-[1,1′-biphenyl]-3-yl)methoxy)-2-formylphenoxy)methyl)nicotinonitrile (0.4 g, 0.56 mmol) in THE (8 mL) and MeOH (2 mL) was added 2-aminoethanol (0.34 g, 5.61 mmol) and glacial AcOH (3.21 uL, 0.06 mmol). The mixture was stirred at r.t. for 0.5 h. After that, the sodium cyanoborohydride (0.1 g, 1.68 mmol) was added. The mixture was stirred at r.t. for 0.5 h. TEA (0.48 mL, 3.49 mmol) and di-tert-butyl dicarbonate (3.65 g, 16.7 mmol) were added and the mixture was stirred at r.t. for 5 min under N₂ atmosphere. The reaction mixture was concentrated under reduced pressure to give a residue. The residue was purified by reverse phase HPLC to give tert-butyl N-[[4-[[3-[3-[1-[2-[tert-butoxycarbonyl(2-hydroxyethyl)amino]ethyl]-4-methyl-2,5-dioxo-3H-1,4-benzodiazepin-7-yl]-2-methyl-phenyl]-2-methyl-phenyl]methoxy]-5-chloro-2-[(5-cyano-3-pyridyl)methoxy]phenyl]methyl]-N-(2-hydroxyethyl)carbamate as a white solid (0.11 g, 15% yield). ¹H NMR (400 MHz, CD3OD): δ8.93 (m, 2H), 8.38 (m, 1H), 7.79 (s, 1H), 7.66-7.59 (m, 2H), 7.49 (d, J=6.8 Hz, 1H), 7.36 (m, 1H), 7.29 (m, 2H), 7.19 (m, 3H), 6.98 (s, 1H), 5.30 (m, 4H), 4.62 (m, 3H), 4.49 (m, 2H), 4.24 (d, J=15.2 Hz, 1H), 3.79 (d, J=14.8 Hz, 1H), 3.54 (m, 6H), 3.28 (s, 3H), 3.22 (m, 1H), 2.17 (s, 3H), 1.95 (s, 3H), 1.43 (m, 18H).

### 5-((4-Chloro-5-((3′-(1-(2-((2-hydroxyethyl)amino)ethyl)-4-methyl-2,5-dioxo-2,3,4,5-tetrahydro-1H-benzo[e][1,4]diazepin-7-yl)-2,2′-dimethyl-[1,1′-biphenyl]-3-yl)methoxy)-2-(((2-hydroxyethyl)amino)methyl)phenoxy)methyl)nicotinonitrile

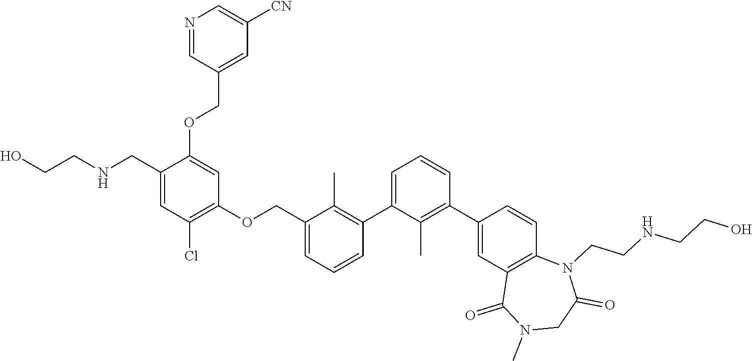 [SMILES: Cc1c(COc2cc(OCc3cncc(C#N)c3)c(CNCCO)cc2Cl)cccc1-c1cccc(-c2ccc3c(c2)C(=O)N(C)CC(=O)N3CCNCCO)c1C]

A mixture of tert-butyl N-[[4-[[3-[3-[1-[2-[tert-butoxycarbonyl(2-hydroxyethyl)amino]ethyl]-4-methyl-2,5-dioxo-3H-1,4-benzodiazepin-7-yl]-2-methyl-phenyl]-2-methyl-phenyl]methoxy]-5-chloro-2-[(5-cyano-3-pyridyl)methoxy]phenyl]methyl]-N-(2-hydroxyethyl)carbamate (0.11 g, 0.11 mmol) in DCM (20 mL) was added TFA (5 mL, 67.5 mmol). The mixture was degassed and purged with N₂ for 3 times, and then the mixture was stirred at r.t. for 20 min under N₂ atmosphere. The aqueous phase were quenched with sodium bicarbonate in saturated aqueous solution to pH=7, and extracted with EtOAc (3×10 mL).

The combined organic layers were washed with brine (50 mL), dried over anhydrous sodium sulfate, filtered and concentrated under reduced pressure to give a residue. The residue was purified by reverse phase HPLC to 5-((4-chloro-5-((3′-(1-(2-((2-hydroxyethyl)amino)ethyl)-4-methyl-2,5-dioxo-2,3,4,5-tetrahydro-1H-benzo[e][1,4]diazepin-7-yl)-2,2′-dimethyl-[1,1′-biphenyl]-3-yl)methoxy)-2-(((2-hydroxyethyl)amino)methyl)phenoxy)methyl)nicotinonitrile as a white solid. (7.03 mg, 8% yield, m/z: 803 [M+H]⁺ observed). ¹H NMR (400 MHz, CD₃OD): δ8.97-8.94 (m, 2H), 8.38 (s, 1H), 7.77 (m, 2H), 7.50 (d, J=7.6 Hz, 1H), 7.38 (m, 2H), 7.31 (m, 2H), 7.18 (d, J=7.2 Hz, 2H), 7.03 (s, 1H) 5.33 (m, 4H), 4.90 (m, 3H), 4.38 (m, 1H), 4.27 (m, 1H), 3.93 (m, 4H), 3.78 (m, 1H), 3.70 (m, 2H), 3.61 (m, 2H), 3.28 (m, 3H), 2.87 (m, 4H), 2.71 (m, 2H), 2.17 (s, 3H), 1.95 (s, 3H).

### Example 9: 5-((4-Chloro-2-(((2-hydroxyethyl)amino)methyl)-5-((3′-(3-(((2-hydroxyethyl)amino)methyl)-4-oxo-4H-quinolizin-8-yl)-2,2′-dimethyl-[1,1′-biphenyl]-3-yl)methoxy)phenoxy)methyl)nicotinonitrile

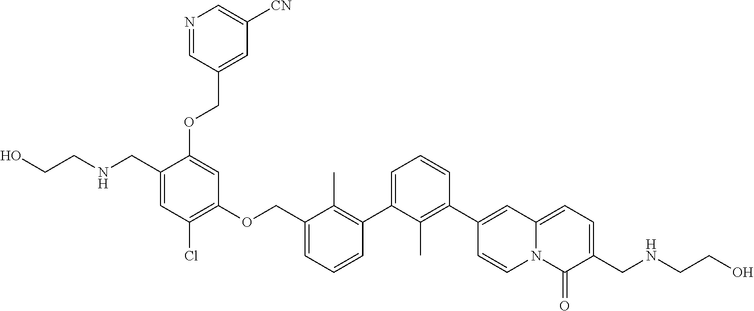 [SMILES: Cc1c(COc2cc(OCc3cncc(C#N)c3)c(CNCCO)cc2Cl)cccc1-c1cccc(-c2ccn3c(=O)c(CNCCO)ccc3c2)c1C]

### Ethyl 8-chloro-4-oxo-4H-quinolizine-3-carboxylate

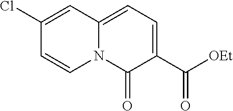 [SMILES: CCOC(=O)c1ccc2cc(Cl)ccn2c1=O]

A solution of 4-chloro-2-methylpyridine (20 g, 157 mmol) in THE (150 mL) was added to 2 N lithium diisopropylamide solution in THF/n-heptane (98 mL, 180 mmol) at −70° C. The mixture was stirred at −60° C. for 0.5 h. Diethyl 2-(ethoxymethylene)malonate (36.4 mL, 180 mmol) was added to the mixture at −60° C. The reaction mixture was stirred at −60° C. for 0.5 hr. The reaction mixture was stirred at r.t. for 3 hr. The residue was diluted with H₂O (100 mL) and extracted with EtOAc (2×50 mL). The combined organic layers were dried over anhydrous sulfate, filtered and concentrated under reduced pressure. The residue was purified by normal phase SiO₂ chromatography (30-100% EtOAc/petroleum ether) to give ethyl 8-chloro-4-oxo-4H-quinolizine-3-carboxylate (26 g, 66% yield). ¹H NMR (400 MHz, DMSO-d₆): δ 9.10 (d, J=7.6 Hz, 1H), 8.25 (d, J=8.4 Hz, 1H), 8.13 (d, J=2.4 Hz, 1H), 7.42 (dd, J=8.0 Hz, J=2.4 Hz 1H), 6.78 (d, J=8.4 Hz, 1H), 4.22 (q, J=7.2 Hz, 2H), 1.27 (t, J=7.2 Hz, 3H).

### 8-Chloro-3-(hydroxymethyl)-4H-quinolizin-4-one

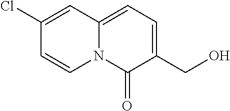 [SMILES: O=c1c(CO)ccc2cc(Cl)ccn12]

To a solution of ethyl 8-chloro-4-oxo-4H-quinolizine-3-carboxylate (6 g, 23.8 mmol) in THE (100 mL) was added 1 N DIBAL-H solution in Toluene (71.5 mL) dropwise at −40° C. The mixture was stirred at −40° C. for 1 hr. The reaction mixture was stirred at r.t. for 2 hr. The reaction mixture was quenched by addition 1 N HCl solution in H₂O (100 mL) at 0° C., diluted with H₂O (100 mL) and extracted with EtOAc (2×50 mL). The combined organic layers were dried over anhydrous sulfate, filtered and concentrated under reduced pressure to give the crude product 8-chloro-3-(hydroxymethyl)-4H-quinolizin-4-one (4 g, crude) as yellow solid. ¹H NMR (400 MHz, DMSO-d₆): δ 8.84 (d, J=8 Hz, 1H), 7.91 (d, J=2 Hz, 1H), 7.82 (d, J=7.6 Hz, 1H), 7.11 (dd, J=7.6 Hz, J=2.4 Hz 1H), 6.81 (d, J=8 Hz, 1H), 5.17 (t, J=5.2 Hz, 1H), 4.48 (t, J=4.8 Hz, 2H).

### 8-Chloro-4-oxo-4H-quinolizine-3-carbaldehyde

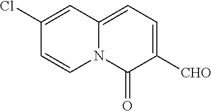 [SMILES: O=Cc1ccc2cc(Cl)ccn2c1=O]

To a solution of 8-chloro-3-(hydroxymethyl)-4H-quinolizin-4-one (4 g, 19.1 mmol) in CHCl₃ (50 mL) was added manganese dioxide (16.6 g, 191 mmol). The mixture was stirred at r.t. for 12 hr. The reaction mixture was filtered and concentrated. The residue was washed with petroleum ether/EtOAc (30 mL/10 mL) to give 8-chloro-4-oxo-4H-quinolizine-3-carbaldehyde (1.85 g, 47% yield) as a yellow solid. ¹H NMR (400 MHz, CDCl₃): δ 10.36 (s, 1H), 8.24 (d, J=8 Hz, 1H), 8.16 (d, J=8 Hz, 1H), 7.53 (s, 1H), 7.13 (d, J=8 Hz, 1H), 6.56 (d, J=8 Hz, 1H).

### 8-(3-Bromo-2-methylphenyl)-4-oxo-4H-quinolizine-3-carbaldehyde

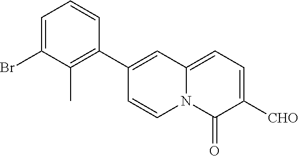 [SMILES: Cc1c(Br)cccc1-c1ccn2c(=O)c(C=O)ccc2c1]

To a solution of 8-chloro-4-oxo-4H-quinolizine-3-carbaldehyde (900 mg, 4.33 mmol) and (3-bromo-2-methylphenyl)boronic acid (466 mg, 2.17 mmol) in dioxane (15 mL) and H₂O (2 mL) was added 1,1′-bis(diphenylphosphino)ferrocene-palladium(ii) dichloride dichloromethane complex (354 mg, 0.43 mmol) and potassium carbonate (1.20 g, 8.67 mmol). The mixture was stirred at 80° C. for 1 hr. The reaction mixture was diluted with H₂O (20 mL) and extracted with EtOAc (2×15 mL). The combined organic layers were dried over anhydrous sulfate, filtered and concentrated under reduced pressure. The residue was purified by normal phase SiO₂ chromatography (0-50% EtOAc/petroleum ether) to give 8-(3-bromo-2-methylphenyl)-4-oxo-4H-quinolizine-3-carbaldehyde (700 mg, 24% yield, m/z: 342 [M]⁺, 344 [M+2]⁺ observed) as a yellow solid.

### 5-((4-Chloro-2-formyl-5-((3′-(3-formyl-4-oxo-4H-quinolizin-8-yl)-2,2′-dimethyl-[1,1′-biphenyl]-3-yl)methoxy)phenoxy)methyl)nicotinonitrile

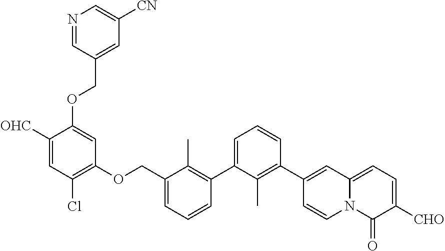 [SMILES: Cc1c(COc2cc(OCc3cncc(C#N)c3)c(C=O)cc2Cl)cccc1-c1cccc(-c2ccn3c(=O)c(C=O)ccc3c2)c1C]

To a solution of 8-(3-bromo-2-methylphenyl)-4-oxo-4H-quinolizine-3-carbaldehyde (300 mg, 0.88 mmol) and 5-((4-chloro-2-formyl-5-((2-methyl-3-(4,4,5,5-tetramethyl-1,3,2-dioxaborolan-2-yl)benzyl)oxy)phenoxy)methyl)nicotinonitrile (682 mg, 1.32 mmol) in THE (5 mL) and H₂O (1 mL) was added 1,1′-bis(diphenylphosphino)ferrocene-palladium(ii) dichloride dichloromethane complex (71.6 mg, 0.087 mmol) and potassium carbonate (242 mg, 1.75 mmol). The mixture was stirred at 80° C. for 1 hr. The reaction mixture was diluted with H₂O (10 mL) and extracted with EtOAc (2×15 mL). The combined organic layers were dried over anhydrous sulfate, filtered and concentrated. The residue was purified by normal phase SiO₂ chromatography (0-50% EtOAc/petroleum ether) to give 5-((4-chloro-2-formyl-5-((3′-(3-formyl-4-oxo-4H-quinolizin-8-yl)-2,2′-dimethyl-[1,1′-biphenyl]-3-yl)methoxy)phenoxy)methyl) nicotinonitrile (300 mg, 52% yield) as a brown solid. m/z: 654 [M+H]⁺ observed.

### 5-((4-Chloro-2-(((2-hydroxyethyl)amino)methyl)-5-((3′-(3-(((2-hydroxyethyl)amino)methyl)-4-oxo-4H-quinolizin-8-yl)-2,2′-dimethyl-[1,1′-biphenyl]-3-yl)methoxy)phenoxy)methyl)nicotinonitrile

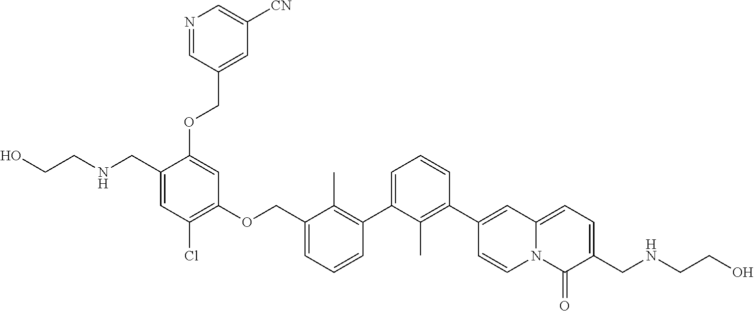 [SMILES: Cc1c(COc2cc(OCc3cncc(C#N)c3)c(CNCCO)cc2Cl)cccc1-c1cccc(-c2ccn3c(=O)c(CNCCO)ccc3c2)c1C]

To a solution of 5-((4-chloro-2-formyl-5-((3′-(3-formyl-4-oxo-4H-quinolizin-8-yl)-2,2′-dimethyl-[1,1′-biphenyl]-3-yl)methoxy)phenoxy)methyl)nicotinonitrile (140 mg, 0.21 mmol) and 2-aminoethanol (0.064 mL, 1.07 mmol) in THE (5 mL) was added glacial AcOH (0.012 mL, 0.21 mmol). The mixture was stirred at r.t. for 12 hr. Sodium cyanoborohydride (26.9 mg, 0.43 mmol) was added and the mixture was stirred at 50° C. for 3 hr. The reaction mixture was filtered and concentrated. The residue was purified by reverse phase HPLC to give 5-((4-chloro-2-(((2-hydroxyethyl)amino)methyl)-5-((3′-(3-(((2-hydroxyethyl)amino)methyl)-4-oxo-4H-quinolizin-8-yl)-2,2′-dimethyl-[1,1′-biphenyl]-3-yl)methoxy)phenoxy)methyl)nicotinonitrile (4.19 mg) as a yellow solid. m/z: 744 [M+H]⁺ observed. ¹H NMR (400 MHz, CD₃OD) δ9.12 (d, J=8 Hz, 1H), 8.93 (d, J=1.6 Hz, 1H), 8.89 (d, J=1.6 Hz, 1H), 8.34 (s, 1H), 7.89 (d, J=8 Hz, 1H), 7.72 (d, J=1.6 Hz, 1H), 7.48 (t, J=7.8 Hz, 1H), 7.40-7.38 (m, 2H), 7.33 (s, 1H), 7.30-7.26 (m, 2H), 7.23-7.20 (m, 1H), 7.15 (d, J=7.2 Hz, 1H), 7.01-6.97 (m, 2H), 5.29 (s, 2H), 5.27 (s, 2H), 3.95 (s, 2H), 3.76 (s, 2H), 3.71 (t, J=5.6 Hz, 2H), 3.63 (t, J=5.6 Hz, 2H), 2.80 (t, J=5.6 Hz, 2H), 2.69 (t, J=5.6 Hz, 2H), 2.16 (s, 3H), 2.00 (s, 3H).

### Example 10: (S)-2-(((8-(3′-((4-((((S)-2-Carboxy-1-hydroxypropan-2-yl)amino)methyl)-2-chloro-5-((5-cyanopyridin-3-yl)methoxy)phenoxy)methyl)-2,2′-dimethyl-[1,1′-biphenyl]-3-yl)-4-oxo-4H-pyrido[1,2-a]pyrimidin-3-yl)methyl)amino)-3-hydroxy-2-methylpropanoic acid

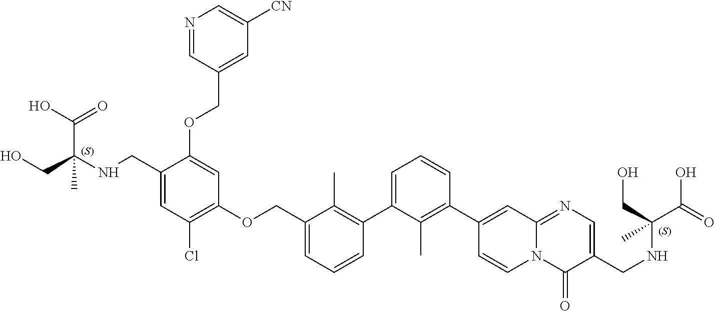

### (S)-2-(((8-(3′-((4-((((S)-2-Carboxy-1-hydroxypropan-2-yl)amino)methyl)-2-chloro-5-((5-cyanopyridin-3-yl)methoxy)phenoxy)methyl)-2,2′-dimethyl-[1,1′-biphenyl]-3-yl)-4-oxo-4H-pyrido[1,2-a]pyrimidin-3-yl)methyl)amino)-3-hydroxy-2-methylpropanoic acid

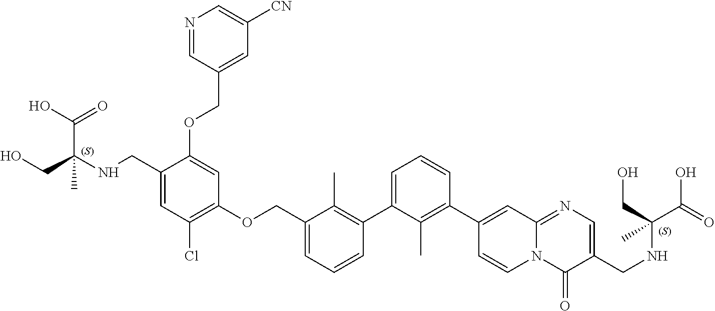

To a mixture of 5-[[4-chloro-2-formyl-5-[[3-[3-(3-formyl-4-oxo-pyrido[1,2-a]pyrimidin-8-yl)-2-methyl-phenyl]-2-methyl-phenyl]methoxy]phenoxy]methyl]pyridine-3-carbonitrile (0.3 g, 0.46 mmol) in MeOH (5 mL) and THF (5 mL) was added (S)-2-amino-3-hydroxypropanoic acid (0.24 g, 2.3 mmol) and glacial HOAc (46 umol, 2.6 uL). The mixture was stirred at r.t. for 1 h. Sodium cyanoborohydride (0.14 g, 2.3 mmol) was added and the mixture was stirred at 25° C. for 0.5 h. The reaction mixture was filtered and the filtrate was purified by reverse phase HPLC to afford (S)-2-(((8-(3′-((4-((((S)-1-carboxy-2-hydroxyethyl)amino)methyl)-2-chloro-5-((5-cyanopyridin-3-yl)methoxy)phenoxy)methyl)-2,2′-dimethyl-[1,1′-biphenyl]-3-yl)-4-oxo-4H-pyrido[1,2-a]pyrimidin-3-yl)methyl)amino)-3-hydroxypropanoic acid as a white solid (8.71 mg). m/z: 833 [M+H]⁺ observed. ¹H NMR (400 MHz, DMSO-d₆): δ 9.00 (m, 3H), 8.50 (s, 1H), 8.44 (s, 1H), 7.70 (s, 1H), 7.49 (m, 3H), 7.45 (m, 2H), 7.29 (m, 1H), 7.23 (m, 1H), 7.15 (m, 2H), 5.33 (m, 4H), 3.94 (m, 5H), 2.09 (s, 3H), 1.94 (s, 3H). ¹H NMR (400 MHz, CD₃OD): δ. 9.19 (d, J=7.6 Hz, 1H), 9.35 (d, J=14.4 Hz, 2H), 8.60 (s, 1H), 8.41 (s, 1H), 7.75 (s, 1H), 7.66 (s, 1H), 7.53 (m, 2H), 7.43 (m, 2H), 7.30 (m, 2H), 7.19 (d, J=7.6 Hz, 1H), 7.08 (s, 1H), 5.37 (s, 2H), 5.30 (s, 2H), 4.57 (m, 3H), 4.30 (m, 3H), 4.18 (m, 1H), 4.01 (m, 1H), 3.92 (m, 1H), 3.88 (m, 1H), 3.81 (m, 1H), 4.63 (m, 1H), 4.48 (m, 1H), 2.15 (s, 3H), 2.01 (s, 3H).

### Example 11: (S)-1-((8-(3′-((4-(((S)-2-Carboxypiperidin-1-yl)methyl)-2-chloro-5-((5-cyanopyridin-3-yl)methoxy)phenoxy)methyl)-2,2′-dimethyl-[1,1′-biphenyl]-3-yl)-4-oxo-4H-pyrido[1,2-a]pyrimidin-3-yl)methyl)piperidine-2-carboxylic acid

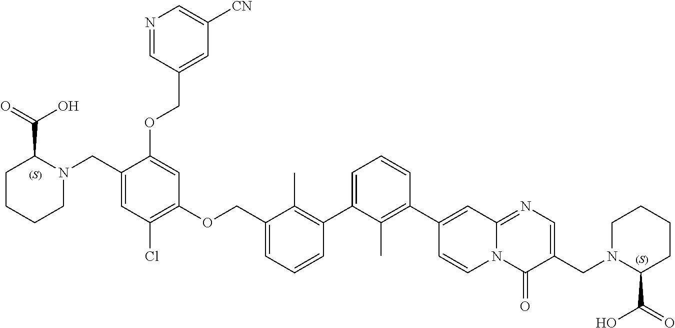

To a mixture of 5-[[4-chloro-2-formyl-5-[[3-[3-(3-formyl-4-oxo-pyrido[1,2-a]pyrimidin-8-yl)-2-methyl-phenyl]-2-methyl-phenyl]methoxy]phenoxy]methyl]pyridine-3-carbonitrile (0.25 g, 0.38 mmol) and (S)-piperidine-2-carboxylic acid (0.5 g, 3.8 mmol) in EtOH (6 mL) was added glacial HOAc (0.038 mmol, 2.2 uL). The mixture was stirred at r.t. for 1 h.

Sodium cyanoborohydride (0.24 g, 3.82 mmol) was added and the mixture was stirred at r.t. for 12 h. The reaction mixture was filtered and the filtrate was purified by reverse phase HPLC directly to afford (S)-1-((8-(3′-((4-(((S)-2-carboxypiperidin-1-yl)methyl)-2-chloro-5-((5-cyanopyridin-3-yl)methoxy)phenoxy)methyl)-2,2′-dimethyl-[1,1′-biphenyl]-3-yl)-4-oxo-4H-pyrido[1,2-a]pyrimidin-3-yl)methyl)piperidine-2-carboxylic acid as a white solid (15.96 mg). m/z: 831 [M+H]⁺ observed. ¹H NMR (400 MHz, CD₃OD): δ9.19 (d, J=7.2 Hz, 1H), 8.96 (s, 1H), 8.90 (s, 1H), 8.49 (s, 1H), 8.41 (s, 1H), 7.70 (s, 1H), 7.49 (m, 3H), 7.41 (m, 2H), 7.27 (m, 2H), 7.18 (m, 1H), 7.08 (s, 1H), 5.36 (m, 4H), 4.58 (m, 2H), 4.44 (t, J=7.6 Hz, 2H), 4.30 (m, 1H), 3.50 (m, 2H), 3.15 (m, 1H), 2.94 (m, 1H), 2.27 (m, 5H), 2.03 (s, 3H), 1.60 (m, 11H).

### Example 12: 5-((4-Chloro-5-((2,2′-dimethyl-3′-(4-oxo-3-(((((S)-5-oxopyrrolidin-2-yl)methyl)amino)methyl)-4H-pyrido[1,2-a]pyrimidin-8-yl)-[1,1′-biphenyl]-3-yl)methoxy)-2-(((((S)-5-oxopyrrolidin-2-yl)methyl)amino)methyl)phenoxy)methyl)nicotinonitrile

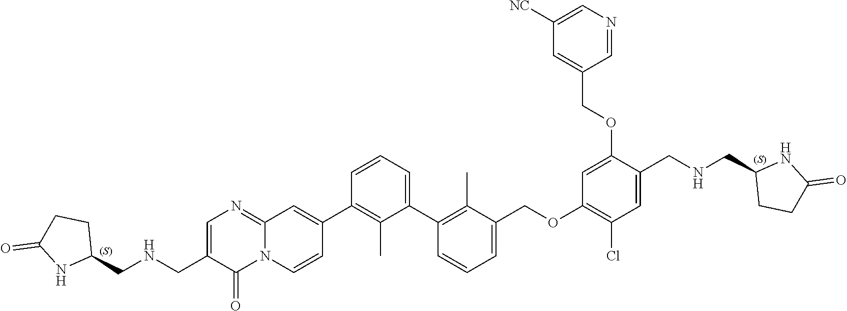

To a mixture of 5-((4-chloro-2-formyl-5-((3′-(3-formyl-4-oxo-4H-pyrido[1,2-a]pyrimidin-8-yl)-2,2′-dimethyl-[1,1′-biphenyl]-3-yl)methoxy)phenoxy)methyl)nicotinonitrile (0.2 g, 0.3 mmol) and (S)-5-(aminomethyl)pyrrolidin-2-one as a hydrochloride salt (0.46 g, 3 mmol) in THE (20 mL) and MeOH (20 mL) was added glacial HOAc (30.5 umol, 1.75 uL). The mixture was stirred at r.t. for 12 hours. Sodium cyanoborohydride (0.38 g, 6 mmol) was added and the mixture was stirred at r.t. for 1 min. The mixture was filtered and concentrated to give the residue. The residue was purified by reverse phase HPLC to afford 5-((4-chloro-5-((2,2′-dimethyl-3′-(4-oxo-3-(((((S)-5-oxopyrrolidin-2-yl)methyl)amino)methyl)-4H-pyrido[1,2-a]pyrimidin-8-yl)-[1,1′-biphenyl]-3-yl)methoxy)-2-(((((S)-5-oxopyrrolidin-2-yl)methyl)amino)methyl)phenoxy)methyl)nicotinonitrile as a white solid (14.09 mg). m/z: 851 [M+H]⁺ observed. ¹H NMR (400 MHz, CD₃OD): δ. 9.12 (d, J=7.6 Hz, 1H), 8.90 (m, 2H), 8.40 (s, 1H), 8.31 (s, 1H), 7.64 (s, 1H), 7.44 (m, 4H), 7.30 (m, 3H), 7.39 (d, J=7.2 Hz, 1H), 6.96 (s, 1H), 5.28 (d, J=8.4 Hz, 4H), 3.82 (m, 6H), 2.68 (m, 4H), 2.29 (m, 6H), 2.16 (s, 3H), 2.00 (m, 3H), 1.80 (m, 2H).

### Example 13: 5-((4-Chloro-5-((2,2′-dimethyl-3′-(4-oxo-3-(((((R)-5-oxopyrrolidin-2-yl)methyl)amino)methyl)-4H-pyrido[1,2-a]pyrimidin-8-yl)-[1,1′-biphenyl]-3-yl)methoxy)-2-(((((R)-5-oxopyrrolidin-2-yl)methyl)amino)methyl)phenoxy)methyl)nicotinonitrile

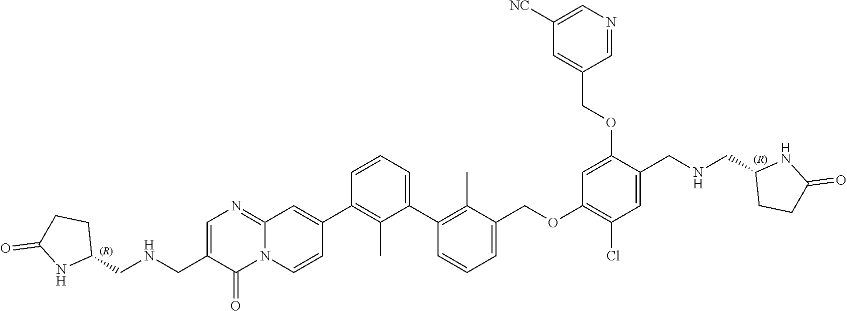

To a mixture of 5-((4-chloro-2-formyl-5-((3′-(3-formyl-4-oxo-4H-pyrido[1,2-a]pyrimidin-8-yl)-2,2′-dimethyl-[1,1′-biphenyl]-3-yl)methoxy)phenoxy)methyl)nicotinonitrile (0.8 g, 1.2 mmol) and (R)-5-(aminomethyl)pyrrolidin-2-one as a hydrochloride salt (1.84 g, 12.2 mmol) in THE (10 mL) and MeOH (10 mL) was added glacial HOAc (0.12 mmol, 7 uL) and 4A molecular sieve (0.5 g). The mixture was stirred at r.t. for 12 hours. Sodium cyanoborohydride (1.53 g, 24.4 mmol) was added and the mixture was stirred at r.t. for 1 min. The mixture was filtered and concentrated to give the residue. The residue was purified by reverse phase HPLC to afford 5-((4-chloro-5-((2,2′-dimethyl-3′-(4-oxo-3-(((((R)-5-oxopyrrolidin-2-yl)methyl)amino)methyl)-4H-pyrido[1,2-a]pyrimidin-8-yl)-[1,1′-biphenyl]-3-yl)methoxy)-2-(((((R)-5-oxopyrrolidin-2-yl)methyl)amino)methyl)phenoxy)methyl)nicotinonitrile as a white solid. (75.08 mg). m/z: 851 [M+H]⁺ observed. ¹H NMR (400 MHz, CD₃OD): δ. 9.11 (d, J=8 Hz, 1H), 8.90 (m, 2H), 8.39 (s, 1H), 8.31 (s, 1H), 7.63 (s, 1H), 7.44 (m, 4H), 7.35 (s, 1H), 7.27 (m, 2H), 7.15 (d, J=7.2 Hz, 1H), 6.96 (s, 1H), 5.27 (m, 4H), 3.84 (m, 3H), 3.76 (m, 3H), 2.68 (m, 4H), 2.29 (m, 6H), 2.15 (s, 3H), 2.00 (s, 3H), 1.76 (m, 2H).

### Example 14: 5-((4-Chloro-5-((2-chloro-3′-(3-(((2-hydroxyethyl)amino)methyl)-4-oxo-4H-pyrido[1,2-a]pyrimidin-8-yl)-2′-methyl-[1,1′-biphenyl]-3-yl)methoxy)-2-(((2-hydroxyethyl)amino)methyl)phenoxy)methyl)nicotinonitrile

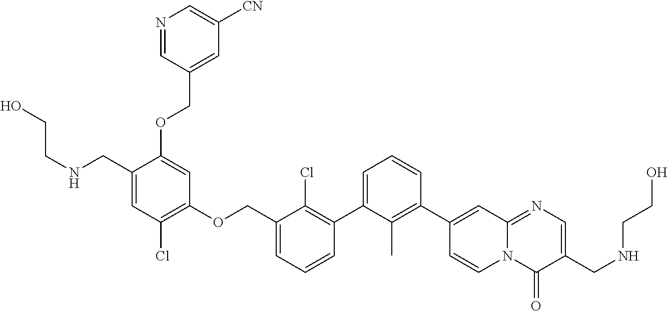 [SMILES: Cc1c(-c2ccn3c(=O)c(CNCCO)cnc3c2)cccc1-c1cccc(COc2cc(OCc3cncc(C#N)c3)c(CNCCO)cc2Cl)c1Cl]

### 4-((3-Bromo-2-chlorobenzyl)oxy)-5-chloro-2-hydroxybenzaldehyde

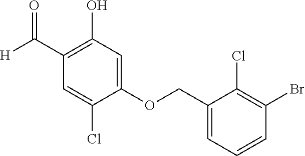 [SMILES: O=Cc1cc(Cl)c(OCc2cccc(Br)c2Cl)cc1O]

To a mixture of 5-chloro-2,4-dihydroxy-benzaldehyde (3.19 g, 18.5 mmol) in acetonitrile (167 mL) was added sodium hydrogen carbonate (4.43 g, 52.8 mmol) and potassium iodide (0.59 g, 3.55 mmol). After that, the mixture was stirred at 50° C. for 0.5 hr under N₂, and then cooled to r.t. A solution of 1-bromo-3-(bromomethyl)-2-chloro-benzene (5 g, 17.6 mmol) in acetonitrile (33.3 mL) was added dropwise. The mixture was stirred at 80° C. for 5 hr under N₂. To the reaction mixture was added water (80 ml) at r.t. and concentrated under reduced pressure. The residue was cooled to 5° C. and allowed to stay for 0.5 hr. The suspension was filtered and the filter solid was washed with cooled acetonitrile (3×10 mL) and water (3×30 mL) to give 4-((3-bromo-2-chlorobenzyl)oxy)-5-chloro-2-hydroxybenzaldehyde as a yellow solid (7 g, crude). ¹H NMR (400 MHz, DMSO-d₆): δ 9.99 (s, 1H), 7.81-7.73 (m, 1H), 7.59 (d, J=6.8 Hz, 1H), 7.47 (s, 1H), 7.35 (t, J=7.8 Hz, 1H), 6.42 (s, 1H), 5.2 (s, 2H).

### 5-((5-((3-Bromo-2-chlorobenzyl)oxy)-4-chloro-2-formylphenoxy)methyl)nicotinonitrile

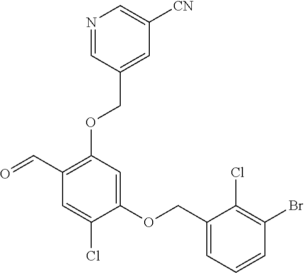 [SMILES: N#Cc1cncc(COc2cc(OCc3cccc(Br)c3Cl)c(Cl)cc2C=O)c1]

To a solution of 4-((3-bromo-2-chlorobenzyl)oxy)-5-chloro-2-hydroxybenzaldehyde (6 g, 16 mmol) in DMF (90 mL) was added potassium iodide (0.86 g, 5.19 mmol) and cesium carbonate (15.6 g, 47.9 mmol). The mixture was stirred at r.t. for 0.5 hr under N₂, 5-(chloromethyl)nicotinonitrile as hydrochloride salt (4.58 g, 24.3 mmol) was added and the mixture was stirred at 50° C. for 15 hr under N₂ atmosphere. The mixture was concentrated to about 45 mL. The residue was cooled to 10° C. and to the mixture was added water (50 mL) with stirring (more solid precipitated). The suspension was filtered and the filter solid was washed with water (3×10 mL), petroleum ether (20 mL). The resultant filter solid was triturated with petroleum ether/EtOAc (14 mL/7 mL) at r.t. for 30 min. The suspension was filtered to give 5-((5-((3-bromo-2-chlorobenzyl)oxy)-4-chloro-2-formylphenoxy)methyl)nicotinonitrile as a yellow solid. (4 g, 51% yield). ¹H NMR (400 MHz, DMSO-d₆): δ 10.23 (s, 1H), 9.02 (dd, J₁=3.2 Hz, J₂=6.4 Hz, 2H), 8.54 (s, 1H), 7.83 (d, J=8 Hz, 1H), 7.74 (s, 1H), 7.68 (d, J=6.8 Hz, 1H), 7.38 (t, J=8 Hz, 1H), 7.23 (s, 1H), 5.48 (s, 2H), 5.47 (s, 2H).

### 8-(2-Methyl-3-(4,4,5,5-tetramethyl-1,3,2-dioxaborolan-2-yl)phenyl)-4-oxo-4H-pyrido[1,2-a]pyrimidine-3-carbaldehyde

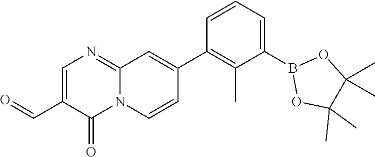 [SMILES: Cc1c(B2OC(C)(C)C(C)(C)O2)cccc1-c1ccn2c(=O)c(C=O)cnc2c1]

A mixture of 8-(3-bromo-2-methylphenyl)-4-oxo-4H-pyrido[1,2-a]pyrimidine-3-carbaldehyde (1 g, 2.9 mmol), 4,4,4′,4′,5,5,5′,5′-octamethyl-2,2′-bi(1,3,2-dioxaborolane) (2.2 g, 8.74 mmol) and potassium acetate (0.86 g, 8.74 mmol) in dioxane (30 mL) was degassed with vacuum/nitrogen cycle (3 times). To the mixture was added ditert-butyl(cyclopentyl)phosphane-dichloropalladium-iron (0.19 mg, 0.29 mmol) and then the mixture was stirred at 80° C. for 2 hr under N₂ atmosphere. To the reaction mixture was added water (10 mL) and extracted with ethyl acetate (3×30 mL). The combined organic layers were washed with brine (20 mL), dried over anhydrous sodium sulfate, filtered and concentrated under reduced pressure. The residue was purified by normal phase SiO₂ chromatography (0˜100% Ethyl acetate/Petroleum ether gradient) to give 8-(2-methyl-3-(4,4,5,5-tetramethyl-1,3,2-dioxaborolan-2-yl)phenyl)-4-oxo-4H-pyrido[1,2-a]pyrimidine-3-carbaldehyde as a yellow oil (0.86 g, 76% yield). ¹H NMR (400 MHz, DMSO-d₆): δ 10.19 (s, 1H), 9.23 (d, J=7.2 Hz, 1H), 8.77 (s, 1H), 7.85 (s, 1H), 7.78 (d, J=7.2 Hz, 1H), 7.66 (dd, J₁=1.6 Hz, J₂=7.2 Hz, 1H), 7.48-7.43 (m, 1H), 7.41-7.35 (m, 1H), 2.46 (s, 3H), 1.33 (s, 12H).

### 5-((4-Chloro-5-((2-chloro-3′-(3-formyl-4-oxo-4H-pyrido[1,2-a]pyrimidin-8-yl)-2′-methyl-[1,1′-biphenyl]-3-yl)methoxy)-2-formylphenoxy)methyl)nicotinonitrile

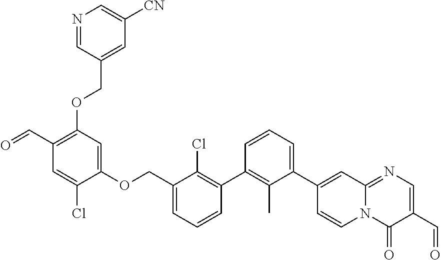 [SMILES: Cc1c(-c2ccn3c(=O)c(C=O)cnc3c2)cccc1-c1cccc(COc2cc(OCc3cncc(C#N)c3)c(C=O)cc2Cl)c1Cl]

To the mixture of 5-((5-((3-bromo-2-chlorobenzyl)oxy)-4-chloro-2-formylphenoxy)methyl)nicotinonitrile (0.96 g, 1.95 mmol), 8-[2-methyl-3-(4,4,5,5-tetramethyl-1,3,2-dioxaborolan-2-yl)phenyl]-4-oxo-pyrido[1,2-a]pyrimidine-3-carbaldehyde (0.76 g, 1.95 mmol) and potassium carbonate (0.81 g, 5.84 mmol) in THE (6 mL) and water (2 mL) was added 1,1′-bis(diphenylphosphino)ferrocene-palladium(ii) dichloride dichloromethane complex (0.16 g, 0.2 mmol). The mixture was degassed with vacuum/nitrogen cycle (3 times), and then stirred at 80° C. for 1 hr under N₂ atmosphere. The reaction mixture was separated, and the aqueous phase was extracted with ethyl acetate (3×10 mL). The combined organic phase was washed with brine (5 mL), dried with anhydrous sodium sulfate, filtered and concentrated in vacuum. The residue was purified by normal phase SiO₂ chromatography (0˜100% Ethyl acetate/Petroleum ether gradient) to give 5-((4-chloro-5-((2-chloro-3′-(3-formyl-4-oxo-4H-pyrido[1,2-a]pyrimidin-8-yl)-2′-methyl-[1,1′-biphenyl]-3-yl)methoxy)-2-formylphenoxy)methyl)nicotinonitrile as a yellow solid (0.5 g, 38% yield). ¹H NMR (400 MHz, CDCl₃): δ 10.32 (s, 1H), 10.22 (s, 1H), 9.26 (d, J=7.2 Hz, 1H), 8.85 (d, J=14 Hz, 3H), 8.02 (s, 1H), 7.89-7.87 (m, 1H), 7.78 (s, 1H), 7.69 (d, J=7.6 Hz, 1H), 7.43-7.37 (m, 3H), 7.32 (d, J=6.8 Hz, 1H), 7.25 (d, J=7.6 Hz, 2H), 6.6 (s, 1H), 5.31 (s, 2H), 5.18 (m, 2H), 2.03 (s, 3H).

### 5-((4-Chloro-5-((2-chloro-3′-(3-(((2-hydroxyethyl)amino)methyl)-4-oxo-4H-pyrido[1,2-a]pyrimidin-8-yl)-2′-methyl-[1,1′-biphenyl]-3-yl)methoxy)-2-(((2-hydroxyethyl)amino)methyl)phenoxy)methyl)nicotinonitrile

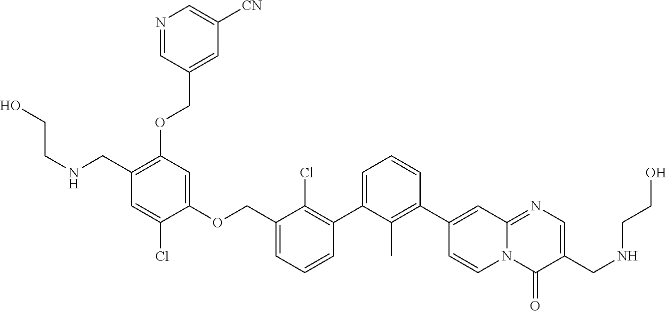 [SMILES: Cc1c(-c2ccn3c(=O)c(CNCCO)cnc3c2)cccc1-c1cccc(COc2cc(OCc3cncc(C#N)c3)c(CNCCO)cc2Cl)c1Cl]

To a solution of 5-((4-chloro-5-((2-chloro-3′-(3-formyl-4-oxo-4H-pyrido[1,2-a]pyrimidin-8-yl)-2′-methyl-[1,1′-biphenyl]-3-yl)methoxy)-2-formylphenoxy)methyl)nicotinonitrile (0.3 g, 0.44 mmol) in THE (10 mL) and MeOH (10 mL) was added 2-aminoethanol (4.44 mmol, 0.27 mL) and glacial acetic acid (0.44 mmol, 25.4 uL) under N₂ atmosphere. The mixture was stirred at r.t. for 12 hr under N₂ atmosphere. Then sodium triacetoxyborohydride (0.94 g, 4.44 mmol) was added and stirred at r.t. for 3 min under N₂ atmosphere. The mixture was concentrated to about 5 mL. The residue was purified by reverse phase HPLC to afford 5-((4-chloro-5-((2-chloro-3′-(3-(((2-hydroxyethyl)amino)methyl)-4-oxo-4H-pyrido[1,2-a]pyrimidin-8-yl)-2′-methyl-[1,1′-biphenyl]-3-yl)methoxy)-2-(((2-hydroxyethyl)amino)methyl)phenoxy)methyl)nicotinonitrile as a formate salt as a white solid (66.4 mg). m/z: 765 [M+H]⁺ observed. ¹H NMR (400 MHz, CD₃OD): δ9.17 (d, J=7.2 Hz, 1H), 8.95 (s, 1H), 8.91 (s, 1H), 8.50 (s, 3H), 8.38 (s, 1H), 7.74-7.73 (m, 2H), 7.54-7.50 (m, 2H), 7.48-7.43 (m, 3H), 7.36 (d, J=7.6 Hz 1H), 7.32 (dd, J₁=5.2 Hz, J₂=2.8 Hz, 1H), 7.04 (s, 1H), 5.41 (s, 2H), 5.36 (s, 2H), 4.24 (s, 2H), 4.17 (s, 2H), 3.83 (t, J=4.8 Hz, 2H), 3.76 (t, J=4.8 Hz, 2H), 3.15 (s, 2H), 3.05 (s, 2H), 2.09 (s, 3H).

### Example 15: 8,8′-(2,2′-Dimethyl-[1,1′-biphenyl]-3,3′-diyl)bis(3-(((2-hydroxyethyl)amino)methyl)-4H-pyrido[1,2-a]pyrimidin-4-one)

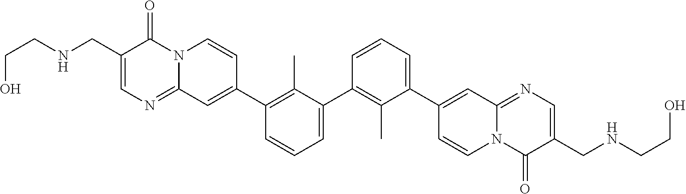 [SMILES: Cc1c(-c2ccn3c(=O)c(CNCCO)cnc3c2)cccc1-c1cccc(-c2ccn3c(=O)c(CNCCO)cnc3c2)c1C]

### 8-(3-Bromo-2-methylphenyl)-3-(((2-hydroxyethyl)amino)methyl)-4H-pyrido[1,2-a]pyrimidin-4-onemethyl

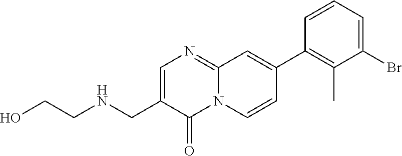 [SMILES: Cc1c(Br)cccc1-c1ccn2c(=O)c(CNCCO)cnc2c1]

To a solution of 8-(3-bromo-2-methylphenyl)-4-oxo-4H-pyrido[1,2-a]pyrimidine-3-carbaldehyde (1.5 g, 4.37 mmol) and 2-aminoethanol (0.53 mL, 8.74 mmol) in THE (20 mL) was added glacial AcOH (0.25 mL, 4.37 mmol). The mixture was stirred at r.t. for 50 min. Sodium cyanoborohydride (0.55 g, 8.74 mmol) was added and the mixture was stirred at r.t. for 10 min to give 8-(3-bromo-2-methylphenyl)-3-(((2-hydroxyethyl)amino)methyl)-4H-pyrido[1,2-a]pyrimidin-4-one as a mixture solution in THE as brown liquid which was used directly. m/z: 388 [M]⁺, 390 [M+2]⁺ observed.

### tert-Butyl ((8-(3-bromo-2-methylphenyl)-4-oxo-4H-pyrido[1,2-a]pyrimidin-3-yl)methyl)(2-hydroxyethyl)carbamate

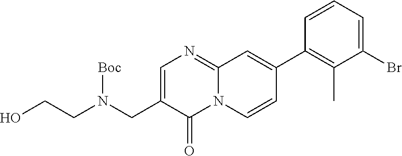 [SMILES: Cc1c(Br)cccc1-c1ccn2c(=O)c(CN(CCO)C(=O)OC(C)(C)C)cnc2c1]

To the reaction solution of 8-(3-bromo-2-methylphenyl)-3-(((2-hydroxyethyl)amino) methyl)-4H-pyrido[1,2-a]pyrimidin-4-one in THE was added MeOH (20 mL), then triethylamine (1.22 mL, 8.76 mmol) and di-tert-butyl dicarbonate (2.01 mL, 8.76 mmol) were added. The mixture was stirred at r.t. for 1 hr. The reaction mixture was concentrated under reduced pressure to remove solvent. The residue was diluted with H₂O (30 mL) and extracted with EtOAc (2×20 mL). The combined organic layers were dried over sodium sulfate, filtered and concentrated. The residue was purified by normal phase SiO₂ chromatography (0-60% EtOAc/petroleum ether) to give tert-butyl((8-(3-bromo-2-methylphenyl)-4-oxo-4H-pyrido[1,2-a]pyrimidin-3-yl)methyl)(2-hydroxyethyl)carbamate (1.2 g, 56% yield for two steps) as a yellow oil. ¹H NMR (400 MHz, CDCl₃) δ 9.11 (d, J=7.6 Hz, 1H), 8.56-8.41 (m, 1H), 7.70 (d, J=7.6 Hz, 1H), 7.60 (s, 1H), 7.26-7.16 (m, 3H), 4.52 (br s, 2H), 3.84 (br s, 2H), 3.68 (br m, 2H), 2.41 (s, 3H), 1.50 (s, 9H).

### tert-Butyl (2-hydroxyethyl)((8-(2-methyl-3-(4,4,5,5-tetramethyl-1,3,2-dioxaborolan-2-yl)phenyl)-4-oxo-4H-pyrido[1,2-a]pyrimidin-3-yl)methyl)carbamate

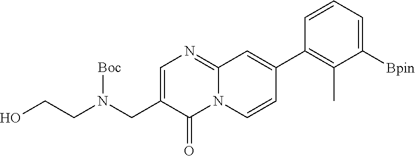

To a solution of tert-butyl ((8-(3-bromo-2-methylphenyl)-4-oxo-4H-pyrido[1,2-a]pyrimidin-3-yl)methyl)(2-hydroxyethyl)carbamate (600 mg, 1.23 mmol) and 4,4,5,5-tetramethyl-2-(4,4,5,5-tetramethyl-1,3,2-dioxaborolan-2-yl)-1,3,2-dioxaborolane (624 mg, 2.46 mmol) in dioxane (10 mL) was added potassium acetate (362 mg, 3.69 mmol) and 1,1′-bis(diphenylphosphino)ferrocene-palladium(II) dichloride dichloromethane complex (100 mg, 0.12 mmol). The mixture was stirred at 80° C. for 12 hr. The reaction mixture was filtered and concentrated. The residue was purified by normal phase SiO₂ chromatography (10-25% 10 EtOAc/petroleum ether) to give tert-butyl (2-hydroxyethyl)((8-(2-methyl-3-(4,4,5,5-tetramethyl-1,3,2-dioxaborolan-2-yl)phenyl)-4-oxo-4H-pyrido[1,2-a]pyrimidin-3-yl)methyl)carbamate (350 mg, 53% yield) as a yellow solid. ¹H NMR (400 MHz, CDCl₃) δ 9.07 (d, J=7.6 Hz, 1H), 8.52-8.38 (m, 1H), 7.87 (dd, J=6 Hz, J=2.8 Hz, 1H), 7.58 (s, 1H), 7.32-7.27 (m, 2H), 7.16 (d, J=6.8 Hz, 1H), 4.49 (br s, 2H), 3.81 (br s, 2H), 3.70-3.60 15 (br m, 2H), 2.49 (s, 3H), 1.60 (s, 9H), 1.36 (s, 12H).

### Di-tert-butyl ((8,8′-(2,2′-dimethyl-[1,1′-biphenyl]-3,3′-diyl)bis(4-oxo-4H-pyrido[1,2-a]pyrimidine-8,3-diyl))bis(methylene))bis((2-hydroxyethyl)carbamate)

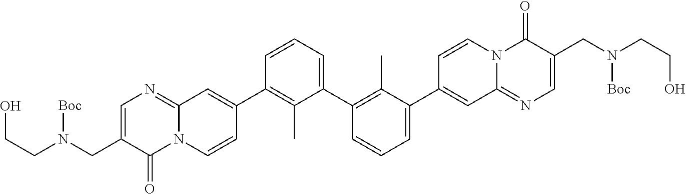 [SMILES: Cc1c(-c2ccn3c(=O)c(CN(CCO)C(=O)OC(C)(C)C)cnc3c2)cccc1-c1cccc(-c2ccn3c(=O)c(CN(CCO)C(=O)OC(C)(C)C)cnc3c2)c1C]

To a solution of tert-butyl ((8-(3-bromo-2-methylphenyl)-4-oxo-4H-pyrido[1,2-a]pyrimidin-3-yl)methyl)(2-hydroxyethyl)carbamate (328 mg, 0.67 mmol) and tert-butyl (2-hydroxyethyl)((8-(2-methyl-3-(4,4,5,5-tetramethyl-1,3,2-dioxaborolan-2-yl)phenyl)-4-oxo-4H-pyrido[1,2-a]pyrimidin-3-yl)methyl)carbamate (300 mg, 0.56 mmol) in THE (5 mL) and H₂O (2 mL) was added 1,1′-bis(diphenylphosphino)ferrocene-palladium(ii) dichloride dichloromethane complex (45.7 mg, 0.056 mmol) and potassium carbonate (232 mg, 1.68 mmol). The mixture was stirred at 80° C. for 0.5 hr. The reaction mixture was concentrated under reduced pressure to remove solvent. The residue was treated with EtOAc (10 mL) and extracted with EtOAc (2×10 mL). The combined organic layers were dried over anhydrous sulfate, filtered and concentrated. The residue was purified by normal phase SiO₂ chromatography (50-100% EtOAc/petroleum ether) to give di-tert-butyl ((8,8′-(2,2′-dimethyl-[1,1′-biphenyl]-3,3′-diyl)bis(4-oxo-4H-pyrido[1,2-a]pyrimidine-8,3-diyl))bis(methylene))bis((2-hydroxyethyl)carbamate) as a white solid (250 mg, 55% yield). m/z: 817 [M+H]⁺ observed.

### 8,8′-(2,2′-Dimethyl-[1,1′-biphenyl]-3,3′-diyl)bis(3-(((2-hydroxyethyl)amino)methyl)-4H-pyrido[1,2-a]pyrimidin-4-one))

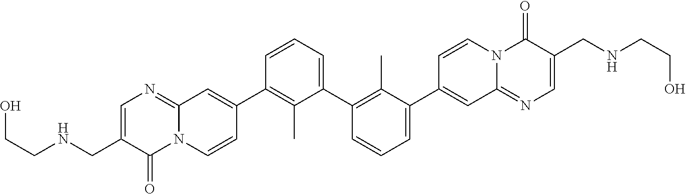 [SMILES: Cc1c(-c2ccn3c(=O)c(CNCCO)cnc3c2)cccc1-c1cccc(-c2ccn3c(=O)c(CNCCO)cnc3c2)c1C]

To a solution of di-tert-butyl ((8,8′-(2,2′-dimethyl-[1,1′-biphenyl]-3,3′-diyl)bis(4-oxo-4H-pyrido[1,2-a]pyrimidine-8,3-diyl))bis(methylene))bis((2-hydroxyethyl)carbamate) (240 mg, 0.29 mmol) in dioxane (5 mL) was added 4 N HCl solution in dioxane (2.5 mL). The mixture was stirred at r.t. for 30 min. The reaction mixture was concentrated. The residue was purified by reverse phase HPLC to give 8,8′-(2,2′-dimethyl-[1,1′-biphenyl]-3,3′-diyl)bis(3-(((2-hydroxyethyl)amino)methyl)-4H-pyrido[1,2-a]pyrimidin-4-one) as hydrochloride salt as a white solid (139 mg, 72% yield). m/z: 617 [M+H]⁺ observed. ¹H NMR (400 MHz, D20) 6 9.11 (d, J=7.6 Hz, 2H), 8.46 (s, 2H), 7.84 (d, J=1.6 Hz, 2H), 7.72 (dd, J=7.6 Hz, J=1.6 Hz, 2H), 7.39-7.33 (m, 4H), 7.25 (dd, J=6.4 Hz, J=2.4 Hz, 2H), 4.26 (s, 4H), 3.76 (t, J=5.2 Hz, 4H), 3.17 (t, J=5.2 Hz, 4H), 1.90 (s, 6H).

### Example 16: 5-((4-chloro-5-((2-cyano-3′-(3-(((2-hydroxyethyl)amino)methyl)-4-oxo-4H-pyrido[1,2-a]pyrimidin-8-yl)-2′-methyl-[1,1′-biphenyl]-3-yl)methoxy)-2-(((2-hydroxyethyl)amino)methyl)phenoxy)methyl)nicotinonitrile

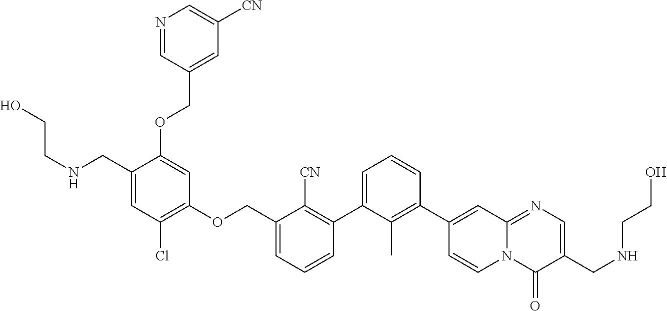 [SMILES: Cc1c(-c2ccn3c(=O)c(CNCCO)cnc3c2)cccc1-c1cccc(COc2cc(OCc3cncc(C#N)c3)c(CNCCO)cc2Cl)c1C#N]

### 2-Bromo-6-((2-chloro-4-formyl-5-hydroxyphenoxy)methyl)benzonitrile

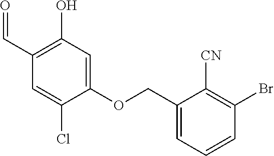 [SMILES: N#Cc1c(Br)cccc1COc1cc(O)c(C=O)cc1Cl]

To a mixture of 5-chloro-2,4-dihydroxybenzaldehyde (2 g, 11.6 mmol) in MeCN(10 mL) was added sodium bicarbonate (1.95 g, 23.2 mmol) in one portion under N₂. The mixture was stirred at r.t. for 0.5 hr. To the mixture was added 2-bromo-6-(bromomethyl)benzonitrile 5 (3.03 g, 11 mmol) in DMF (10 mL). The mixture was stirred at r.t. for 2 hr. The resulting light yellow solid was collected by filtration. The crude product was dried under vacuum to give 2-bromo-6-((2-chloro-4-formyl-5-hydroxyphenoxy)methyl)benzonitrile as a light yellow solid (2.5 g, 59% yield). ¹H NMR (400 MHz, DMSO-d₆): δ 9.97 (s, 1H), 7.93-7.87 (m, 2H), 7.72-7.66 (m, 2H), 6.47 (s, 1H), 5.31 (s, 2H).

### 5-((5-((3-Bromo-2-cyanobenzyl)oxy)-4-chloro-2-formylphenoxy)methyl)nicotinonitrile

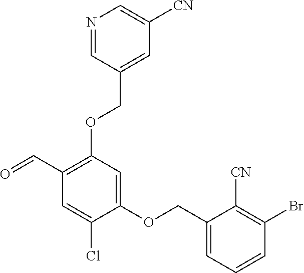 [SMILES: N#Cc1cncc(COc2cc(OCc3cccc(Br)c3C#N)c(Cl)cc2C=O)c1]

To a solution of 2-bromo-6-((2-chloro-4-formyl-5-hydroxyphenoxy)methyl)benzonitrile (2 g, 5.46 mmol) in DMF (30 mL) was added cesium carbonate (5.33 g, 16.4 mmol) and potassium iodide (0.29 g, 1.77 mmol). Then 5-(chloromethyl)nicotinonitrile as a hydrochloride salt (1.57 g, 8.29 mmol) was added and the mixture was stirred at 50° C. for 12 hr under N₂. The mixture was concentrated to about 15 mL. The mixture was cooled to 10° C. and water (40 mL) was added with stirring (more solid precipitated). The suspension was filtered and the filter cake was washed with water (3×5 mL) and petroleum ether (5 mL) to give a yellow solid. The crude product was triturated with petroleum ether/EtOAc (10 mL/5 mL) at r.t. for 30 min. The resulting yellow solid (2.5 g) was collected by filtration. The residue was purified by normal phase SiO₂ chromatography (0-100% EtOAc/petroleum ether) to give 5-((5-((3-bromo-2-cyanobenzyl)oxy)-4-chloro-2-formylphenoxy)methyl)nicotinonitrile as a yellow solid (0.84 g, 32% yield). ¹H NMR (400 MHz, DMSO-d₆): δ 10.24 (s, 1H), 9.02 (dd, J₁=2 Hz, J₂=6.8 Hz, 2H), 8.54 (s, 1H), 7.94 (d, J=7.6 Hz, 1H), 7.78-7.69 (m, 3H), 7.25 (s, 1H), 5.54 (s, 2H), 5.48 (s, 2H).

### 5-((4-Chloro-5-((2-cyano-3′-(3-formyl-4-oxo-4H-pyrido[1,2-a]pyrimidin-8-yl)-2′-methyl-[1,1′-biphenyl]-3-yl)methoxy)-2-formylphenoxy)methyl)nicotinonitrile

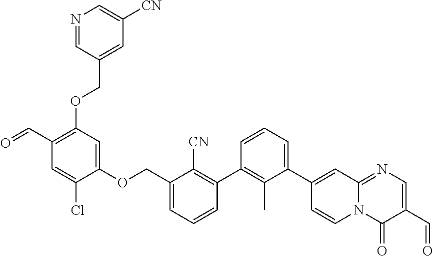 [SMILES: Cc1c(-c2ccn3c(=O)c(C=O)cnc3c2)cccc1-c1cccc(COc2cc(OCc3cncc(C#N)c3)c(C=O)cc2Cl)c1C#N]

To a solution of 5-((5-((3-bromo-2-cyanobenzyl)oxy)-4-chloro-2-formylphenoxy)methyl)nicotinonitrile (0.84 g, 1.74 mmol) in THF/water (3:1, 10 mL) was added 8-(2-methyl-3-(4,4,5,5-tetramethyl-1,3,2-dioxaborolan-2-yl)phenyl)-4-oxo-4H-pyrido[1,2-a]pyrimidine-3-carbaldehyde (0.75 g, 1.91 mmol) and potassium carbonate (0.72 g, 5.22 mmol) under N₂. Then 1,1′-bis(diphenylphosphino)ferrocene-palladium(ii) dichloride dichloromethane complex (0.14 g, 0.17 mmol) was added, and the mixture was stirred at 80° C. for 1 hr under N₂. The aqueous phase was extracted with EtOAc (3×8 mL). The combined organic phase was washed with brine (5 mL), dried with anhydrous sodium sulfate, filtered and concentrated in vacuum. The residue was purified by normal phase SiO₂ chromatography (0-100% EtOAc/petroleum ether) to give 5-((4-chloro-5-((2-cyano-3′-(3-formyl-4-oxo-4H-pyrido[1,2-a]pyrimidin-8-yl)-2′-methyl-[1,1′-biphenyl]-3-yl)methoxy)-2-formylphenoxy)methyl)nicotinonitrile as a yellow solid (0.33 g, 29% yield). ¹H NMR (400 MHz, DMSO-d₆): δ 10.24 (s, 1H), 10.20 (s, 1H), 9.28 (d, J=7.2 Hz, 1H), 9.03 (s, 2H), 8.79 (s, 1H), 8.55 (s, 1H), 7.92 (s, 1H), 7.89 (d, J=7.6 Hz, 1H), 7.85-7.83 (m, 1H), 7.75 (s, 1H), 7.71 (dd, J₁=2 Hz, J₂=7.2 Hz, 1H), 7.65 (d, J=7.2 Hz, 1H), 7.57-7.55 (m, 2H), 7.48-7.46 (m, 1H), 7.32 (s, 1H), 5.6 (s, 2H), 5.5 (s, 2H), 2.13 (s, 3H).

### 5-((4-Chloro-5-((2-cyano-3′-(3-(((2-hydroxyethyl)amino)methyl)-4-oxo-4H-pyrido[1,2-a]pyrimidin-8-yl)-2′-methyl-[1,1′-biphenyl]-3-yl)methoxy)-2-(((2-hydroxyethyl)amino)methyl)phenoxy)methyl)nicotinonitrile

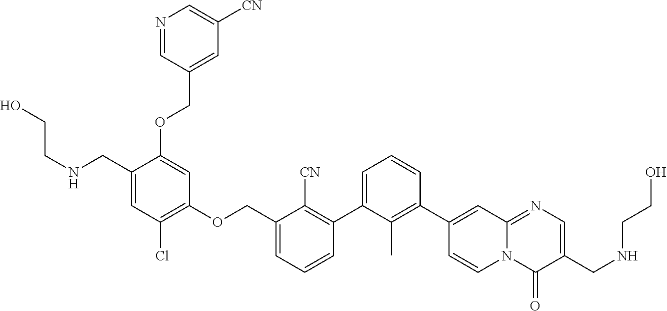 [SMILES: Cc1c(-c2ccn3c(=O)c(CNCCO)cnc3c2)cccc1-c1cccc(COc2cc(OCc3cncc(C#N)c3)c(CNCCO)cc2Cl)c1C#N]

To a solution of 5-((4-chloro-5-((2-cyano-3′-(3-formyl-4-oxo-4H-pyrido[1,2-a]pyrimidin-8-yl)-2′-methyl-[1,1′-biphenyl]-3-yl)methoxy)-2-formylphenoxy)methyl)nicotinonitrile (0.3 g, 0.45 mmol) in THF/MeOH (1:1, 40 mL) was added 2-aminoethanol (4.5 mmol, 0.27 mL) and glacial acetic acid (0.45 mmol, 25 uL) under N₂. The resultant mixture was stirred at r.t. for 12 hr under N₂. Then sodium triacetoxyborohydride (1.24 g, 5.86 mmol) was added and the mixture was stirred at r.t. for 3 min under N₂. The mixture was concentrated to about 5 mL and purified directly by reverse phase HPLC to give 5-((4-chloro-5-((2-cyano-3′-(3-(((2-hydroxyethyl)amino)methyl)-4-oxo-4H-pyrido[1,2-a]pyrimidin-8-yl)-2′-methyl-[1,1′-biphenyl]-3-yl)methoxy)-2-(((2-hydroxyethyl)amino)methyl)phenoxy)methyl)nicotinonitrile as FA salt as a white solid (8.84 mg). m/z: 756 [M+H]⁺ observed. ¹H NMR (400 MHz, CD₃OD): δ9.19 (d, J=7.6 Hz, 1H), 8.96 (s, 1H), 8.92 (s, 1H), 8.51 (s, 2H), 8.40 (s, 1H), 7.84-7.80 (m, 2H), 7.75 (s, 1H) 7.53-7.52 (m, 4H), 7.44-7.40 (m, 1H), 7.14 (s, 1H), 5.48 (s, 2H), 5.39 (s, 2H), 4.26 (s, 2H), 4.19 (s, 2H), 3.83 (t, J=5.2 Hz, 2H), 3.76 (t, J=5.6 Hz, 2H), 3.17 (t, J=5.2 Hz, 2H), 3.06 (t, J=5.6 Hz, 2H), 2.17 (s, 3H).

### Example 17: 5-((4-Chloro-2-(((2-hydroxyethyl)amino)methyl)-5-((3′-(8-(((2-hydroxyethyl)amino)methyl)-4-oxo-4H-pyrido[1,2-a]pyrimidin-3-yl)-2,2′-dimethyl-[1,1′-biphenyl]-3-yl)methoxy)phenoxy)methyl)nicotinonitrile

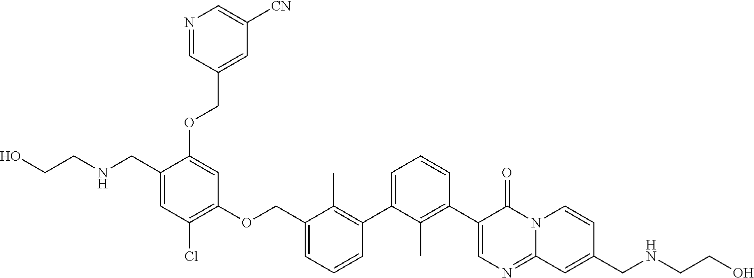 [SMILES: Cc1c(COc2cc(OCc3cncc(C#N)c3)c(CNCCO)cc2Cl)cccc1-c1cccc(-c2cnc3cc(CNCCO)ccn3c2=O)c1C]

### 5-(((4-Bromopyridin-2-yl)amino)methylene)-2,2-dimethyl-1,3-dioxane-4,6-dione

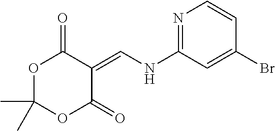 [SMILES: CC1(C)OC(=O)C(=CNc2cc(Br)ccn2)C(=O)O1]

A mixture of 4-bromopyridin-2-amine (23 g, 135 mmol) and 5-(methoxymethylene)-2,2-dimethyl-1,3-dioxane-4,6-dione (25 g, 134 mmol) in isopropyl alcohol (500 mL) was stirred at 110° C. for 45 min, at r.t. for 30 min. The mixture was concentrated under reduced pressure to about 120 mL (more yellow solid was precipitated), cooled to 0° C. and stayed at 0° C. for 30 min. The resulting yellow solid was collected by filtration, washed with ethanol (3×10 mL), petroleum ether (2×50 mL) and dried under reduced pressure to give 5-(((4-bromopyridin-2-yl)amino)methylene)-2,2-dimethyl-1,3-dioxane-4,6-dione (42 g, 89% yield) as a yellow solid. ¹H NMR (400 MHz, CDCl₃): 11.27 (br d, J=12.8 Hz, 1H), 9.37 (d, J=13.2 Hz, 1H), 8.24 (d, J=5.2 Hz, 1H), 7.33 (dd, J=5.2, 1.6 Hz, 1H) 7.26 (m, 1H), 1.76 (s, 6H).

### 8-Bromo-4H-pyrido[1,2-a]pyrimidin-4-one

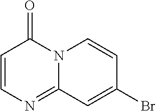 [SMILES: O=c1ccnc2cc(Br)ccn12]

A mixture of 5-(((4-bromopyridin-2-yl)amino)methylene)-2,2-dimethyl-1,3-dioxane-4,6-dione (21 g, 64.2 mmol) in diphenyl ether (180 mL) was stirred at 240° C. for 45 min, at r.t. for 30 min. The mixture was triturated with petroleum ether/cyclohexane mixture (2:1 v/v, 600 mL) at r.t. for 30 min. The resulting brown solid was collected by filtration, washed with petroleum ether (3×50 mL) and dried under reduced pressure to afford 8-bromo-4H-pyrido[1,2-a]pyrimidin-4-one (25 g, 87% yield). ¹H NMR (400 MHz, CDCl₃): δ 8.92 (d, J=7.6 Hz, 1H), 8.26 (d, J=6.4 Hz, 1H), 7.87 (d, J=2.0 Hz, 1H), 7.25 (d, J=2.0 Hz, 1H), 6.47 (d, J=6.4 Hz, 1H).

### 8-Vinyl-4H-pyrido[1,2-a]pyrimidin-4-one

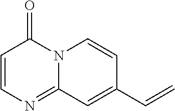

To a solution of 8-bromo-4H-pyrido[1,2-a]pyrimidin-4-one (12.5 g, 55.6 mmol) and potassium vinyltrifluoroborate (10.2 g, 75.8 mmol) in THE (450 mL) and water (50 mL) was added cesium carbonate (46.9 g, 144 mmol) and 1,1′-bis(diphenylphosphino)ferrocene-palladium(ii) dichloride dichloromethane complex (4.6 g, 5.6 mmol). The mixture was stirred at 80° C. under nitrogen gas for 4 hrs. The mixture was filtered through CELITE®, the filtrate was diluted with THE (200 mL), washed with saturated aqueous brine solution (3×80 mL), dried over anhydrous sodium sulfate, filtered and concentrated under reduced pressure. The residue was purified by normal phase SiO₂ chromatography (90-50% petroleum ether/50% dichloromethane in ethyl acetate) to give 8-vinyl-4H-pyrido[1,2-a]pyrimidin-4-one (16 g, 84% yield) as a yellow solid. ¹H NMR (400 MHz, CDCl₃): δ 9.01 (d, J=7.6 Hz, 1H), 8.28 (d, J=6.4 Hz, 1H), 7.50 (s, 1H), 7.30-7.26 (m, 1H), 6.80 (dd, J=17.6, 10.8 Hz, 1H), 6.41 (d, J=6.0 Hz, 1H), 6.10 (d, J=17.6 Hz, 1H), 5.70 (d, J=10.8 Hz, 1H).

### 4-Oxo-4H-pyrido[1,2-a]pyrimidine-8-carbaldehyde

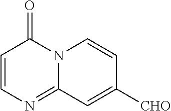 [SMILES: O=Cc1ccn2c(=O)ccnc2c1]

To solution of 8-vinyl-4H-pyrido[1,2-a]pyrimidin-4-one (3 g, 17.4 mmol) in water (50 mL) and THE (100 mL) was added a solution of osmium tetroxide (0.1 M solution in tert-butanol, 9 mL, 0.9 mmol). After stirring at r.t. for 0.5 hr, sodium periodate (9 g, 42 mmol) was added. The mixture was stirred at r.t. for another 2 hrs. The mixture was combined with the two batches at 6.5 g scale. To the mixture was added a solution of sodium thiosulfate pentahydrate (90 g) in water (200 mL) at 5° C. The mixture was stirred at 5° C. for 5 min. The mixture was diluted with THE (500 mL) and separated. The organic phase was washed with saturated aqueous brine solution (3×100 mL), dried over anhydrous sodium sulfate, filtered and concentrated under reduced pressure. The residue was purified by normal phase flash SiO₂ chromatography (90-20% petroleum ether/50% THE in ethyl acetate) to afford 4-oxo-4H-pyrido[1,2-a]pyrimidine-8-carbaldehyde (4.6 g, 28% yield, m/z: 193 [M+H₂O+H]⁺ observed) as a yellow solid.

### 3-Bromo-4-oxo-4H-pyrido[1,2-a]pyrimidine-8-carbaldehyde

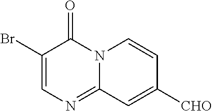 [SMILES: O=Cc1ccn2c(=O)c(Br)cnc2c1]

To a solution of 4-oxo-4H-pyrido[1,2-a]pyrimidine-8-carbaldehyde (2.3 g, 13.2 mmol) in carbon tetrachloride (300 mL) was added NBS (3.2 g, 18 mmol). The mixture was stirred at 90° C. for 1 hr. The mixture was combined and concentrated to about 80 mL). The suspension was cooled to 0° C. and maintained at that temperature for 20 min. The resulting yellow solid was collected by filtration, washed with petroleum ether/ethyl acetate (2:1 (v v), 45 mL), water (3×15 mL) and dried under reduced pressure to give 3-bromo-4-oxo-4H-pyrido[1,2-a]pyrimidine-8-carbaldehyde (5.2 g, 51% yield, m/z: 271 [M+H₂O]⁺, 273 [M+H₂O+2]⁺ observed) as a yellow solid. ¹H NMR (400 MHz, CDCl₃): δ 10.15 (s, 1H), 9.14 (br d, J=7.2 Hz, 1H), 8.70 (s, 1H), 8.10 (s, 1H), 7.58 (br d, J=6.8 Hz, 1H).

### 3-Bromo-8-(((2-hydroxyethyl)amino)methyl)-4H-pyrido[1,2-a]pyrimidin-4-one

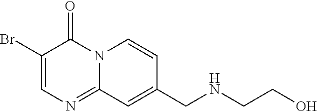 [SMILES: O=c1c(Br)cnc2cc(CNCCO)ccn12]

To solution of 3-bromo-4-oxo-4H-pyrido[1,2-a]pyrimidine-8-carbaldehyde (3.5 g, 13.8 mmol) in 1,2-dichloroethane (250 mL) was added 2-aminoethanol (3.5 mL, 58 mmol), 4A molecular sieve (2 g) and glacial acetic acid (0.8 mL, 14.4 mmol). The mixture was stirred at r.t. for 12 hrs. Then sodium cyanoborohydride (3.5 g, 55.7 mmol) was added. The mixture was stirred at r.t. for 3 hrs. The upper clear liquid was poured out, washed with saturated aqueous brine solution (80 mL), dried over anhydrous sodium sulfate and filtered. The remained dark purple oil was dissolved with THE (200 mL) to give a dark solution which was washed with saturated aqueous brine solution (3×60 mL), dried over anhydrous sodium sulfate and filtered. The above two filtrates were combined and concentrated under reduced pressure to give 3-bromo-8-(((2-hydroxyethyl)amino)methyl)-4H-pyrido[1,2-a]pyrimidin-4-one (5 g, crude) as a dark violet solid which was used in the next step directly.

### tert-Butyl ((3-bromo-4-oxo-4H-pyrido[1,2-a]pyrimidin-8-yl)methyl)(2-hydroxyethyl)carbamate

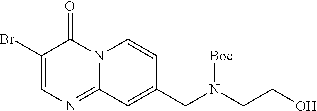 [SMILES: CC(C)(C)OC(=O)N(CCO)Cc1ccn2c(=O)c(Br)cnc2c1]

To solution of 3-bromo-8-(((2-hydroxyethyl)amino)methyl)-4H-pyrido[1,2-a] pyrimidin-4-one (5 g, 16.8 mmol) in THE (350 mL) was added TEA (8 mL, 57.5 mmol) and di-tert-butyl dicarbonate (13 mL, 56.6 mmol). The mixture was stirred at r.t. for 12 hrs. The mixture was diluted with ethyl acetate (50 mL), washed with saturated aqueous brine solution (3×60 mL), dried over anhydrous sodium sulfate, filtered and concentrated under reduced pressure. The residue was purified by normal phase flash SiO₂ chromatography (90-20% petroleum ether/50% THE in ethyl acetate) twice to give 1.26 g of product as a yellow solid which was purified by reverse phase HPLC to afford tert-butyl ((3-bromo-4-oxo-4H-pyrido[1,2-a]pyrimidin-8-yl)methyl) (2-hydroxyethyl)carbamate (704 mg, 13% for two steps, m/z: 398 [M]⁺, 400 [M+2]⁺ observed). ¹H NMR (400 MHz, CDCl₃): δ 9.06 (d, J=6.0 Hz, 1H), 8.57 (s, 1H), 7.51 (s, 1H), 7.14 (d, J=6.8 Hz, 1H), 4.75-4.54 (m, 2H), 3.82 (br s, 2H), 3.60-3.37 (m, 2H), 1.56-1.35 (m, 9H).

### tert-Butyl ((3-(3-bromo-2-methylphenyl)-4-oxo-4H-pyrido[1,2-a]pyrimidin-8-yl)methyl)(2-hydroxyethyl)carbamate

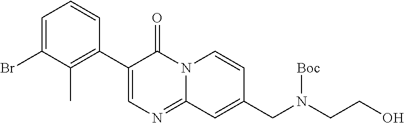 [SMILES: Cc1c(Br)cccc1-c1cnc2cc(CN(CCO)C(=O)OC(C)(C)C)ccn2c1=O]

To a solution of tert-butyl ((3-bromo-4-oxo-4H-pyrido[1,2-a]pyrimidin-8-yl)methyl) (2-hydroxyethyl)carbamate (700 mg, 1.8 mmol) and (3-bromo-2-methylphenyl)boronic acid (400 mg, 1.9 mmol) in water (5 mL), ethanol (10 mL) and toluene (40 mL) was added cesium fluoride (280 mg, 1.8 mmol) and 1,1′-bis(diphenylphosphino)ferrocene-palladium(II) dichloride dichloromethane complex (210 mg, 0.26 mmol) and sodium carbonate (525 mg, 5 mmol). The mixture was stirred at 80° C. under nitrogen gas for 1 hr. The mixture was combined with the batch at 35 mg scale and diluted with THE (20 mL), washed with saturated aqueous brine solution (2×10 mL), dried over anhydrous sulfate, filtered and concentrated under reduced pressure. The residue was purified by normal phase flash SiO₂ chromatography (90-50% petroleum ether/50% THE in ethyl acetate) to give 600 mg of semi-purified product as a yellow solid which was further purified by by reverse phase HPLC to afford tert-butyl ((3-(3-bromo-2-methylphenyl)-4-oxo-4H-pyrido[1,2-a]pyrimidin-8-yl)methyl)(2-hydroxyethyl)carbamate (256 mg, 27% yield, m/z: 488 [M]⁺, 490 [M+2]⁺ observed) as a yellow solid. ¹H NMR (400 MHz, CDCl₃): δ 9.13 (br d, J=6.8 Hz, 1H), 8.29 (s, 1H), 7.61 (d, J=8.4 Hz, 1H), 7.54 (s, 1H), 7.23 (d, J=7.6 Hz, 1H), 7.15-7.12 (m, 2H), 4.76-4.60 (m, 2H), 3.83 (br s, 2H), 3.62-3.41 (m, 2H), 2.34 (s, 3H), 1.56-1.39 (m, 9H).

### tert-Butyl ((3-(3′-((2-chloro-5-((5-cyanopyridin-3-yl)methoxy)-4-formylphenoxy)methyl)-2,2′-dimethyl-[1,1′-biphenyl]-3-yl)-4-oxo-4H-pyrido[1,2-a]pyrimidin-8-yl)methyl)(2-hydroxyethyl)carbamate

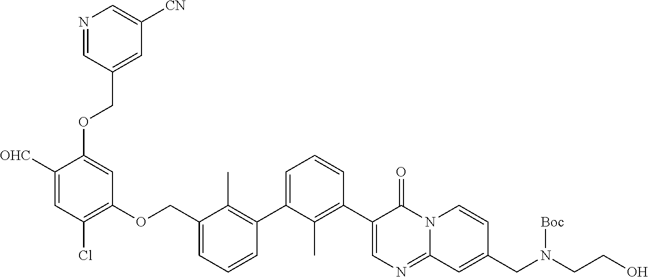 [SMILES: Cc1c(COc2cc(OCc3cncc(C#N)c3)c(C=O)cc2Cl)cccc1-c1cccc(-c2cnc3cc(CN(CCO)C(=O)OC(C)(C)C)ccn3c2=O)c1C]

To a solution of tert-butyl ((3-(3-bromo-2-methylphenyl)-4-oxo-4H-pyrido[1,2-a] pyrimidin-8-yl)methyl)(2-hydroxyethyl)carbamate (256 mg, 0.52 mmol) and 5-((4-chloro-2-formyl-5-((2-methyl-3-(4,4,5,5-tetramethyl-1,3,2-dioxaborolan-2-yl) benzyl)oxy)phenoxy)methyl)nicotinonitrile (320 mg, 0.62 mmol) in THF (25 mL) and water (5 mL) was added potassium carbonate (230 mg, 1.7 mmol) and 1,1′-bis(diphenylphosphino)ferrocene-palladium(ii) dichloride dichloromethane complex (100 mg, 0.12 mmol). The mixture was stirred at 80° C. under nitrogen gas for 5 hrs. The mixture was diluted with THE (10 mL), washed saturated aqueous brine solution (2×10 mL), dried over anhydrous sulfate, filtered and concentrated under reduced pressure. The residue was purified by normal phase flash SiO₂ chromatography (90-33% petroleum ether/50% THE in ethyl acetate) to give tert-butyl ((3-(3′-((2-chloro-5-((5-cyanopyridin-3-yl)methoxy)-4-formylphenoxy) methyl)-2,2′-dimethyl-[1,1′-biphenyl]-3-yl)-4-oxo-4H-pyrido[1,2-a]pyrimidin-8-yl)methyl)(2-hydroxyethyl)carbamate (360 mg, 86% yield) as a yellow solid. ¹H NMR (400 MHz, CDCl₃): δ 10.28 (s, 1H), 9.13 (br d, J=8.0 Hz, 1H), 8.92 (s, 2H), 8.38 (s, 1H), 8.11 (s, 1H), 7.93 (s, 1H), 7.54 (s, 1H), 7.50-7.47 (m, 1H), 7.37-7.28 (m, 3H), 7.25-7.19 (m, 1H), 7.12 (br s, 1H), 6.66 (s, 1H), 5.28 (s, 2H), 5.22 (s, 2H), 4.65 (br s, 2H), 3.83 (br s, 2H), 3.53 (br s, 2H), 2.19 (s, 3H), 1.95 (s, 3H), 1.55-1.45 (m, 9H).

### tert-Butyl ((3-(3′-((2-chloro-5-((5-cyanopyridin-3-yl)methoxy)-4-(((2-hydroxyethyl)amino)methyl)phenoxy)methyl)-2,2′-dimethyl-[1,1′-biphenyl]-3-yl)-4-oxo-4H-pyrido[1,2-a]pyrimidin-8-yl)methyl)(2-hydroxyethyl)carbamate

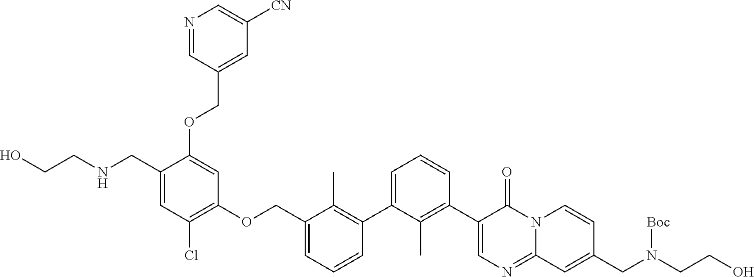 [SMILES: Cc1c(COc2cc(OCc3cncc(C#N)c3)c(CNCCO)cc2Cl)cccc1-c1cccc(-c2cnc3cc(CN(CCO)C(=O)OC(C)(C)C)ccn3c2=O)c1C]

To a solution of tert-butyl ((3-(3′-((2-chloro-5-((5-cyanopyridin-3-yl)methoxy)-4-formylphenoxy)methyl)-2,2′-dimethyl-[1,1′-biphenyl]-3-yl)-4-oxo-4H-pyrido[1,2-a]pyrimidin-8-yl)methyl)(2-hydroxyethyl)carbamate (330 mg, 0.41 mmol) and 2-aminoethanol (0.15 mL, 2.5 mmol) in 1,2-dichloroethane (20 mL) and THE (15 mL) was added 4A molecular sieve (200 mg) and glacial acetic acid (0.024 mL, 0.41 mmol). The mixture was stirred at r.t. under nitrogen gas for 5 hrs. Sodium cyanoborohydride (150 mg, 2.4 mmol) was added and the mixture was stirred at r.t. for 12 hrs. To the mixture was added more 2-aminoethanol (0.15 mL, 2.5 mmol) and sodium cyanoborohydride (150 mg). The mixture was stirred at r.t. for 1 hr. The mixture was combined with the batch at 30 mg scale and diluted with THE (50 mL), washed with saturated aqueous brine solution (2×10 mL), dried over anhydrous sulfate, filtered and concentrated under reduced pressure. The residue was purified by reverse phase HPLC to afford tert-butyl ((3-(3′-((2-chloro-5-((5-cyanopyridin-3-yl) methoxy)-4-(((2-hydroxyethyl)amino)methyl)phenoxy)methyl)-2,2′-dimethyl-[1,1′-biphenyl]-3-yl)-4-oxo-4H-pyrido[1,2-a]pyrimidin-8-yl)methyl)(2-hydroxyethyl) carbamate (200 mg, 53% yield, m/z: 845 [M+H]⁺, 423 [M/2+H]⁺ observed) as a yellow solid.

### 5-((4-Chloro-2-(((2-hydroxyethyl)amino)methyl)-5-((3′-(8-(((2-hydroxyethyl)amino)methyl)-4-oxo-4H-pyrido[1,2-a]pyrimidin-3-yl)-2,2′-dimethyl-[1,1′-biphenyl]-3-yl)methoxy)phenoxy)methyl)nicotinonitrile

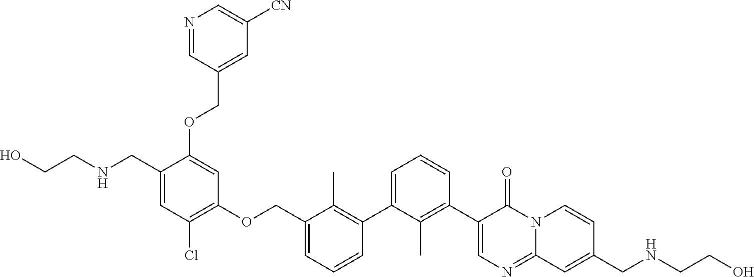 [SMILES: Cc1c(COc2cc(OCc3cncc(C#N)c3)c(CNCCO)cc2Cl)cccc1-c1cccc(-c2cnc3cc(CNCCO)ccn3c2=O)c1C]

To a solution of tert-butyl ((3-(3′-((2-chloro-5-((5-cyanopyridin-3-yl)methoxy)-4-(((2-hydroxyethyl)amino)methyl)phenoxy)methyl)-2,2′-dimethyl-[1,1′-biphenyl]-3-yl)-4-oxo-4H-pyrido[1,2-a]pyrimidin-8-yl)methyl)(2-hydroxyethyl)carbamate (180 mg, 0.21 mmol) in THF (5 mL) at 0° C. was added HCl (4 M solution in EtOAc, 9 mL, 36 mmol). The mixture was stirred at r.t. for 2 hrs (a lot of yellow solid was precipitated). The light yellow solid was collected by filtration and dried under reduced pressure. Then THE (15 mL) and EtOAc (10 mL) were added and the mixture was neutralized with saturated aqueous sodium bicarbonate solution. The mixture was separated and the organic phase was washed with saturated aqueous brine solution (5 mL), dried over anhydrous sulfate, filtered and concentrated under reduced pressure. The residue was purified by reverse phase HPLC to afford 5-((4-chloro-2-(((2-hydroxyethyl)amino)methyl)-5-((3′-(8-(((2-hydroxyethyl)amino)methyl)-4-oxo-4H-pyrido[1,2-a]pyrimidin-3-yl)-2,2′-dimethyl-[1,1′-biphenyl]-3-yl)methoxy)phenoxy)methyl)nicotinonitrile (52 mg, 31% yield, m/z: 745 [M+H]⁺, 373 [M/2+H]⁺ observed) as a light yellow solid. ¹H NMR (400 MHz, CD₃OD): δ9.10 (d, J=7.6 Hz, 1H), 8.93 (d, J=2.0 Hz, 1H), 8.89 (d, J=2.0 Hz, 1H), 8.34 (s, 1H), 8.33 (t, J=2.0 Hz, 1H), 7.73 (d, J=1.2 Hz, 1H), 7.47 (d, J=6.8 Hz, 1H), 7.43 (dd, J=7.6, 1.6 Hz, 1H), 7.36-7.22 (m, 4H), 7.19-7.13 (m, 2H), 6.96 (s, 1H), 5.33-5.22 (m, 4H), 4.02 (s, 2H), 3.75 (s, 2H), 3.71 (t, J=5.6 Hz, 2H), 3.63 (t, J=5.6 Hz, 2H), 2.80 (t, J=5.6 Hz, 2H), 2.68 (t, J=5.4 Hz, 2H), 2.17 (s, 3H), 1.90 (s, 3H).

### Example 18: 5-((4-Chloro-2-(((2-hydroxyethyl)amino)methyl)-5-(((3′-(3-(((2-hydroxyethyl)amino)methyl)-4-oxo-4H-pyrido[1,2-a]pyrimidin-8-yl)-2,2′-dimethyl-[1,1′-biphenyl]-3-yl)oxy)methyl)phenoxy)methyl)nicotinonitrile

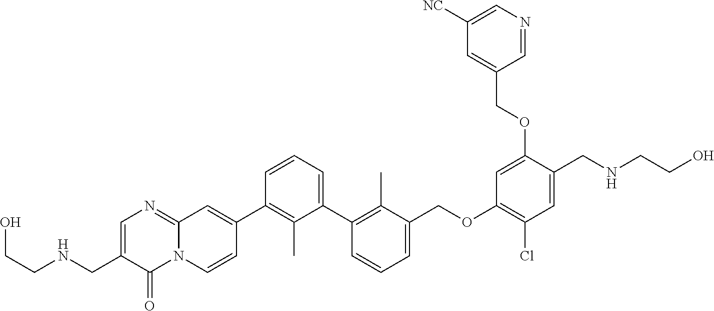 [SMILES: Cc1c(COc2cc(OCc3cncc(C#N)c3)c(CNCCO)cc2Cl)cccc1-c1cccc(-c2ccn3c(=O)c(CNCCO)cnc3c2)c1C]

### Methyl 5-chloro-2-hydroxy-4-methylbenzoate

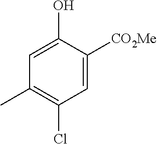

To a solution of methyl 2-hydroxy-4-methyl-benzoate (25 g, 150 mmol) in MeCN (250 mL) was added 1-chloropyrrolidine-2,5-dione (22.1 g, 165 mmol). The mixture was stirred at 40° C. for 12 hr. The reaction mixture was combined with another batch at 20 g scale. Sodium thiosulphate (10 g) was added followed by water (300 mL). The mixture was extracted with ethyl acetate (2×300 mL). The combined organic phase was dried with anhydrous sodium sulfate, filtered and concentrated. The residue was purified by normal phase SiO₂ chromatography (0-5% EtOAc/petroleum ether) to afford methyl 5-chloro-2-hydroxy-4-methylbenzoate (40 g, 41% yield) as a white solid. ¹H NMR (400 MHz, CDCl₃): δ 10.54 (s, 1H), 7.75 (s, 1H), 6.84 (s, 1H), 3.92 (s, 3H), 2.34 (s, 3H).

### Methyl 2-acetoxy-5-chloro-4-methylbenzoate

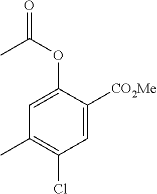 [SMILES: COC(=O)c1cc(Cl)c(C)cc1OC(C)=O]

To a solution of methyl 5-chloro-2-hydroxy-4-methylbenzoate (10 g, 49.9 mmol) in pyridine (100 mL) was added acetyl acetate (14 mL, 149 mmol). The mixture was stirred at r.t. for 12 hr. To the mixture was added water (100 mL). The mixture was extracted with ethyl acetate (2×100 mL). The combined organic phase was dried with anhydrous sodium sulfate, filtered and concentrated. The residue was purified by normal phase SiO₂ chromatography (0-20% EtOAc/petroleum ether) to afford methyl 2-acetoxy-5-chloro-4-methylbenzoate (40 g, 83% yield) as a yellow oil. ¹H NMR (400 MHz, CDCl₃): δ 7.98 (s, 1H), 6.98 (s, 1H), 3.85 (s, 3H), 2.39 (s, 3H), 2.33 (s, 3H).

### Methyl 2-acetoxy-4-(bromomethyl)-5-chlorobenzoate

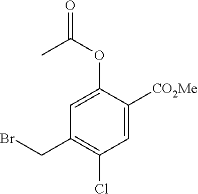 [SMILES: COC(=O)c1cc(Cl)c(CBr)cc1OC(C)=O]

To a solution of methyl 2-acetoxy-5-chloro-4-methylbenzoate (10 g, 41.2 mmol) in CCl₄ (40 mL) was added 1-bromopyrrolidine-2,5-dione (7.33 g, 41.2 mmol) and benzoic peroxyanhydride (1.50 g, 6.18 mmol). The mixture was stirred at 80° C. for 12 h. The reaction mixture was combined with another three batches at same scale. Then sodium thiosulphate (5 g) was added followed by water (200 mL). The mixture was extracted with ethyl acetate (2×200 mL). The combined organic phase was dried with anhydrous sodium sulfate, filtered and concentrated. The residue was purified by normal phase SiO₂ chromatography (0-20% EtOAc/petroleum ether) to afford methyl 2-acetoxy-4-(bromomethyl)-5-chlorobenzoate (30 g, 57% yield) as a yellow oil. ¹H NMR (400 MHz, CDCl₃): δ 8.02 (s, 1H), 7.21 (s, 1H), 4.52 (s, 2H), 3.86 (s, 3H), 2.33 (s, 3H).

### Methyl 4-((3-bromo-2-methylphenoxy)methyl)-5-chloro-2-((2-(trimethylsilyl)ethoxy)methoxy)benzoate

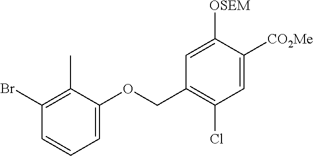

To a solution of methyl 2-acetoxy-4-(bromomethyl)-5-chlorobenzoate (10 g, 31.1 mmol) and 3-bromo-2-methylphenol (8.72 g, 46.7 mmol) in DMF (400 mL) was added sodium iodide (9.32 g, 62.2 mmol) and cesium carbonate (30.4 g, 93.3 mmol). The mixture was stirred at 50° C. for 1 hr. 2-(chloromethoxy)ethyl-trimethylsilane (13.8 mL, 77.8 mmol) was added to the mixture at 0° C. The mixture was stirred at 50° C. for 1 hr. The reaction mixture was combined with another batch at same scale. Water (800 mL) was added and the mixture was extracted with ethyl acetate (2×800 mL). The combined organic phase was dried with anhydrous sodium sulfate, filtered and concentrated. The residue was purified by normal phase SiO₂ chromatography (0-5% EtOAc/petroleum ether) to afford methyl 4-((3-bromo-2-methylphenoxy)methyl)-5-chloro-2-((2-(trimethylsilyl)ethoxy)methoxy)benzoate (13 g) as a yellow liquid. ¹H NMR (400 MHz, CDCl₃): δ 7.84 (s, 1H), 7.57 (s, 1H), 7.21 (d, J=8 Hz, 1H), 7.02 (t, J=8 Hz, 1H), 8.83 (d, J=8.4 Hz, 1H), 5.29 (s, 2H), 5.14 (s, 2H), 3.91 (s, 3H), 3.77 (t, J=8.4 Hz, 2H), 2.44 (s, 3H), 0.91 (t, J=8 Hz, 2H), 0.00 (s, 7H).

### (4-((3-Bromo-2-methylphenoxy)methyl)-5-chloro-2-((2-(trimethylsilyl)ethoxy)methoxy)phenyl)methanol

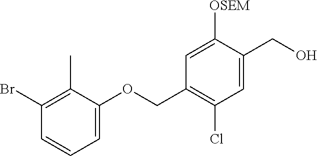

To a solution of methyl 4-((3-bromo-2-methylphenoxy)methyl)-5-chloro-2-((2-(trimethylsilyl)ethoxy)methoxy)benzoate (13 g, 25.2 mmol) in THE (130 mL) was added lithium aluminum hydride (1.15 g, 30.2 mmol) at −20° C. The mixture was stirred at −20° C. for 30 min. To the mixture was added water (100 mL). The mixture was extracted with ethyl acetate (2×100 mL). The combined organic phase was dried with anhydrous sodium sulfate, filtered and concentrated. The residue was purified by normal phase SiO₂ chromatography (0-5% EtOAc/petroleum ether) to afford (4-((3-bromo-2-methylphenoxy)methyl)-5-chloro-2-((2-(trimethylsilyl)ethoxy)methoxy)phenyl)methanol (10 g, 81% yield) as a white solid. ¹H NMR (400 MHz, CDCl₃): δ 7.38 (m, 2H), 7.19 (d, J=8 Hz, 1H), 7.01 (t, J=8 Hz, 1H), 6.84 (d, J=8 Hz, 1H), 5.25 (s, 2H), 5.12 (s, 2H), 4.68 (d, J=6.4 Hz, 2H), 3.73 (t, J=8.4 Hz, 2H), 2.41 (s, 3H), 0.92 (t, J=8.4 Hz, 2H), 0.00 (s, 9H).

### (4-((3-Bromo-2-methylphenoxy)methyl)-5-chloro-2-((2-(trimethylsilyl)ethoxy)methoxy)phenyl)methanol

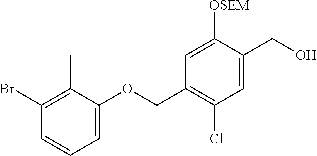

To a solution of (4-((3-bromo-2-methylphenoxy)methyl)-5-chloro-2-((2-(trimethylsilyl)ethoxy)methoxy)phenyl)methanol (10 g, 20.5 mmol) in DCM (100 mL) was added Dess-Martin Periodinate (10.4 g, 24.6 mmol). The mixture was stirred at r.t. for 12 hr. Water (50 mL) was added and the mixture was extracted with ethyl acetate (2×50 mL). The combined organic phase was dried with anhydrous sodium sulfate, filtered and concentrated. The residue was purified by normal phase SiO₂ chromatography (0-10% EtOAc/petroleum ether) to afford (4-((3-bromo-2-methylphenoxy)methyl)-5-chloro-2-((2-(trimethylsilyl)ethoxy)methoxy)phenyl)methanol (8 g, 80% yield) as a yellow solid. ¹H NMR (400 MHz, CDCl₃): δ 10.42 (s, 1H), 7.84 (s, 1H), 7.61 (s, 1H), 7.21 (d, J=8 Hz, 1H), 7.02 (t, J=8 Hz, 1H), 6.82 (d, J=8 Hz, 1H), 5.33 (s, 2H), 5.15 (s, 2H), 3.76 (t, J=8 Hz, 2H), 2.44 (s, 3H), 0.91 (t, J=8.4 Hz, 2H), 0.00 (s, 9H).

### 4-((3-Bromo-2-methylphenoxy)methyl)-5-chloro-2-((2-(trimethylsilyl)ethoxy)methoxy)benzaldehyde

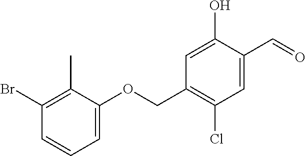 [SMILES: Cc1c(Br)cccc1OCc1cc(O)c(C=O)cc1Cl]

To a solution of (4-((3-bromo-2-methylphenoxy)methyl)-5-chloro-2-((2-(trimethylsilyl)ethoxy)methoxy)phenyl)methanol (3 g, 6.17 mmol) in DCM (15 mL) was added dibromomagnesium;ethoxyethane (3.19 g, 12.4 mmol). The mixture was stirred at r.t. for 1 hr. The reaction mixture was combined with another batch at same scale and was extracted with ethyl acetate (2×50 mL). The combined organic phase was dried with anhydrous sodium sulfate, filtered and concentrated. The residue was purified by normal phase SiO₂ chromatography (0-10% EtOAc/petroleum ether) to afford 4-((3-bromo-2-methylphenoxy)methyl)-5-chloro-2-((2-(trimethylsilyl)ethoxy)methoxy)benzaldehyde (2 g, 30% yield) as a white solid. ¹H NMR (400 MHz, CDCl₃): δ 10.94 (s, 1H), 9.84 (s, 1H), 7.57 (s, 1H), 7.24 (d, J=5.2 Hz, 1H), 7.21 (d, J=8 Hz, 1H), 6.98 (t, J=8 Hz, 1H), 6.78 (d, J=8.4 Hz, 1H), 5.12 (s, 2H), 2.42 (s, 3H).

### 5-((5-((3-Bromo-2-methylphenoxy)methyl)-4-chloro-2-formylphenoxy)methyl)nicotinonitrile

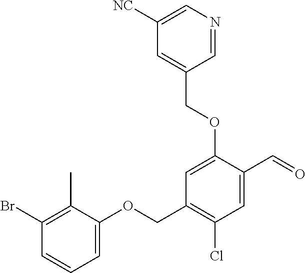 [SMILES: Cc1c(Br)cccc1OCc1cc(OCc2cncc(C#N)c2)c(C=O)cc1Cl]

To a solution of 4-((3-bromo-2-methylphenoxy)methyl)-5-chloro-2-((2-(trimethylsilyl)ethoxy)methoxy)benzaldehyde (0.8 g, 2.25 mmol) and 5-(chloromethyl)nicotinonitrile hydrochloride (0.63 g, 3.37 mmol) in DMF (6 mL) was added sodium iodide (0.84 g, 5.62 mmol) and potassium carbonate (1.55 g, 11.2 mmol). The mixture was stirred at 65° C. for 12 hr. To the mixture was added water (5 mL). The mixture was extracted with ethyl acetate (2×5 mL). The combined organic phase was dried with anhydrous sodium sulfate, filtered and concentrated. The residue was purified by normal phase SiO₂ chromatography (0-50% EtOAc/petroleum ether) to afford 5-((5-((3-bromo-2-methylphenoxy)methyl)-4-chloro-2-formylphenoxy)methyl)nicotinonitrile (0.4 g, 38% yield) as a white solid. ¹H NMR (400 MHz, CDCl₃): δ 10.36 (s, 1H), 8.81 (s, 2H), 7.83 (s, 1H), 7.17 (t, J=6 Hz, 2H), 6.95 (t, J=8 Hz, 1H), 6.69 (d, J=8.4 Hz, 1H), 5.19 (m, 2H), 5.10 (m, 2H), 2.32 (s, 3H).

### 5-((4-Chloro-2-formyl-5-(((3′-(3-formyl-4-oxo-4H-pyrido[1,2-a]pyrimidin-8-yl)-2,2′-dimethyl-[1,1′-biphenyl]-3-yl)oxy)methyl)phenoxy)methyl)nicotinonitrile

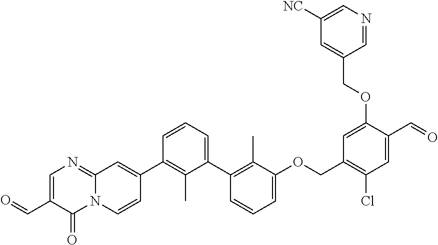 [SMILES: Cc1c(OCc2cc(OCc3cncc(C#N)c3)c(C=O)cc2Cl)cccc1-c1cccc(-c2ccn3c(=O)c(C=O)cnc3c2)c1C]

To a solution of 5-((5-((3-bromo-2-methylphenoxy)methyl)-4-chloro-2-formylphenoxy)methyl)nicotinonitrile (400 mg, 848 umol) and 8-(2-methyl-3-(4,4,5,5-tetramethyl-1,3,2-dioxaborolan-2-yl)phenyl)-4-oxo-4H-pyrido[1,2-a]pyrimidine-3-carbaldehyde (496 mg, 127 umol) in THF (30 mL) and H₂O (10 mL) was added potassium phosphate (360 mg, 170 umol) and [2-(2-aminophenyl)phenyl]-chloro-palladium;bis(1-adamantyl)-butyl-phosphane (57 mg, 84.8 umol). The mixture was stirred at 75° C. for 2 hr. To the mixture was added water (50 mL). The mixture was extracted with ethyl acetate (2×50 mL). The combined organic phase was dried with anhydrous sodium sulfate, filtered and concentrated. The residue was purified by normal phase SiO₂ chromatography (0-80% EtOAc/petroleum ether) to afford 5-((4-chloro-2-formyl-5-(((3′-(3-formyl-4-oxo-4H-pyrido[1,2-a]pyrimidin-8-yl)-2,2′-dimethyl-[1,1′-biphenyl]-3-yl)oxy)methyl)phenoxy)methyl)nicotinonitrile (400 mg, 72% yield) as a yellow solid. ¹H NMR (400 MHz, DMSO-d₆): δ 10.44 (s, 1H), 10.25 (s, 1H), 9.30 (d, J=6.8 Hz, 1H), 9.05 (s, 1H), 8.84 (s, 2H), 8.57 (s, 1H), 7.99 (s, 1H), 7.83 (s, 1H), 7.78 (s, J=8 Hz, 1H), 7.66 (s, 1H), 7.50 (m, 2H), 7.32 (d, J=8 Hz, 2H), 7.18 (d, J=8 Hz, 1H), 6.89 (d, 1H), 5.51 (s, 2H), 5.30 (s, 2H), 2.06 (s, 3H), 2.01 (s, 3H).

### 5-((4-Chloro-2-(((2-hydroxyethyl)amino)methyl)-5-(((3′-(3-(((2-hydroxyethyl)amino)methyl)-4-oxo-4H-pyrido[1,2-a]pyrimidin-8-yl)-2,2′-dimethyl-[1,1′-biphenyl]-3-yl)oxy)methyl)phenoxy)methyl)nicotinonitrile

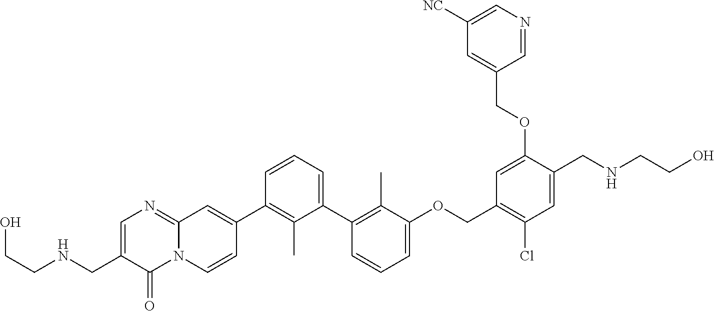 [SMILES: Cc1c(OCc2cc(OCc3cncc(C#N)c3)c(CNCCO)cc2Cl)cccc1-c1cccc(-c2ccn3c(=O)c(CNCCO)cnc3c2)c1C]

To a solution of 5-((4-chloro-2-formyl-5-(((3′-(3-formyl-4-oxo-4H-pyrido[1,2-a]pyrimidin-8-yl)-2,2′-dimethyl-[1,1′-biphenyl]-3-yl)oxy)methyl)phenoxy)methyl)nicotinonitrile (200 mg, 305 umol) and 2-aminoethanol (185 uL, 305 umol) in THE (1 mL) and MeOH (1 mL) was added acetic acid (9 uL, 152 umol). The mixture was stirred at r.t. for 13 hr. Sodium cyanoborohydride (38.4 mg, 610 umol) was added to the mixture. The mixture was stirred at r.t. for 5 hr. The reaction mixture was concentrated under reduced pressure was purified by reverse phase HPLC to afford 5-((4-chloro-2-(((2-hydroxyethyl)amino)methyl)-5-(((3′-(3-(((2-hydroxyethyl)amino)methyl)-4-oxo-4H-pyrido[1,2-a]pyrimidin-8-yl)-2,2′-dimethyl-[1,1′-biphenyl]-3-yl)oxy)methyl)phenoxy)methyl)nicotinonitrile (50.12 mg, 21.51% yield, m/z: 745 [M+H]⁺ observed) as a white solid. ¹H NMR (400 MHz, CD₃OD): δ9.14 (d, J=7.2 Hz, 1H), 8.89 (m, 2H), 8.42 (s, 1H), 8.31 (s, 1H), 7.67 (d, J=0.8 Hz, 1H), 7.61 (m, 4H), 7.32 (s, 1H), 7.26 (m, 2H), 7.02 (d, J=8 Hz, 1H), 6.81 (d, J=7.6 Hz, 1H), 5.31 (s, 2H), 5.23 (s, 2H), 3.88 (d, J=8 Hz, 4H), 3.71 (m, 4H), 2.81 (t, J=5.2 Hz, 2H), 2.74 (t, J=5.6 Hz, 2H), 2.05 (s, 3H), 2.01 (s, 3H).

### Example 19: 5-((4-Chloro-5-(((2,2′-dimethyl-3′-(4-oxo-3-(((((S)-5-oxopyrrolidin-2-yl)methyl)amino)methyl)-4H-pyrido[1,2-a]pyrimidin-8-yl)-[1,1′-biphenyl]-3-yl)oxy)methyl)-2-(((((S)-5-oxopyrrolidin-2-yl)methyl)amino)methyl)phenoxy)methyl)nicotinonitrile

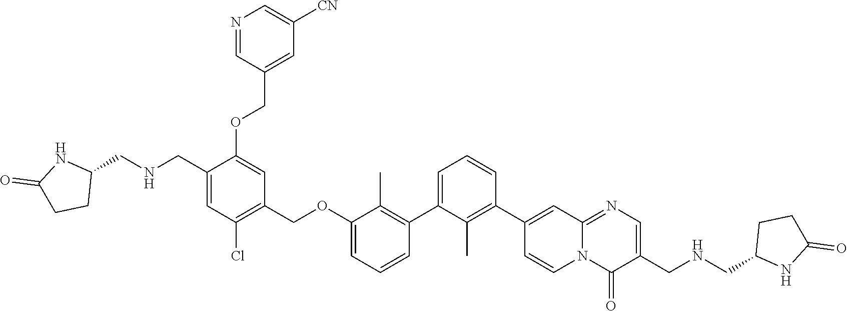 [SMILES: Cc1c(OCc2cc(OCc3cncc(C#N)c3)c(CNC[C@@H]3CCC(=O)N3)cc2Cl)cccc1-c1cccc(-c2ccn3c(=O)c(CNC[C@@H]4CCC(=O)N4)cnc3c2)c1C]

To a suspension of 5-((4-chloro-2-formyl-5-(((3′-(3-formyl-4-oxo-4H-pyrido[1,2-a]pyrimidin-8-yl)-2,2′-dimethyl-[1,1′-biphenyl]-3-yl)oxy)methyl)phenoxy)methyl)nicotinonitrile (170 mg, 259 umol), (S)-5-(aminomethyl)pyrrolidin-2-one hydrochloride (391 mg, 260 umol) and molecular sieves pack 4 Å powder (50 mg) in THE (1 mL) and MeOH (1 mL) was added acetic acid (14.8 uL, 259 umol). The mixture was stirred at r.t. for 2 hr. Sodium cyanoborohydride (48.9 mg, 778 umol) was added and the mixture was stirred at r.t. for 1 hr. The reaction mixture was filtered and concentrated under reduced pressure to give a residue. The residue was purified by reverse phase HPLC to afford 5-((4-chloro-5-(((2,2′-dimethyl-3′-(4-oxo-3-(((((S)-5-oxopyrrolidin-2-yl)methyl)amino)methyl)-4H-pyrido[1,2-a]pyrimidin-8-yl)-[1,1′-biphenyl]-3-yl)oxy)methyl)-2-(((((S)-5-oxopyrrolidin-2-yl)methyl)amino)methyl)phenoxy)methyl)nicotinonitrile (56.8 mg, 25.36% yield, m/z: 851 [M+H]⁺ observed) as a white solid. ¹H NMR (400 MHz, CD₃OD): δ9.13 (d, J=7.6 Hz, 1H), 8.93 (m, 2H), 8.41 (m, 1H), 8.28 (s, 1H), 7.66 (s, 1H), 7.44 (m, 4H), 7.30 (s, 1H), 7.25 (m, 2H), 7.03 (m, 1H), 6.81 (d, J=7.6 Hz, 1H), 5.29 (m, 2H), 5.23 (m, 2H), 3.84 (m, 6H), 2.72 (m, 4H), 2.30 (m, 6H), 2.02 (s, 6H), 1.82 (m, 2H).

### Example 20: 5-((4-Chloro-5-((2,2′-dimethyl-3′-(4-methyl-2,5-dioxo-1-(2-((((S)-5-oxopyrrolidin-2-yl)methyl)amino)ethyl)-2,3,4,5-tetrahydro-1H-benzo[e][1,4]diazepin-7-yl)-[1,1′-biphenyl]-3-yl)methoxy)-2-(((((S)-5-oxopyrrolidin-2-yl)methyl)amino)methyl)phenoxy)methyl)nicotinonitrile

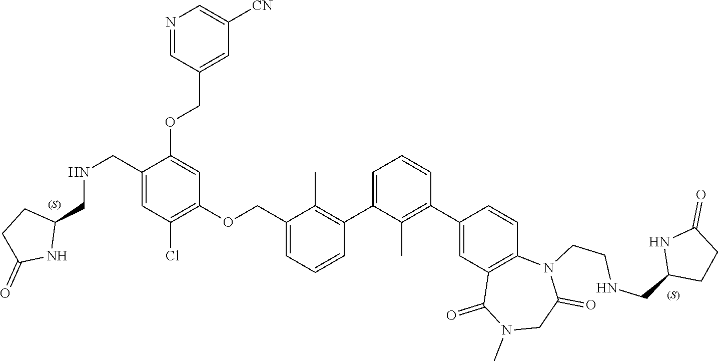

To a mixture of 5-((4-chloro-5-((2,2′-dimethyl-3′-(4-methyl-2,5-dioxo-1-(2-oxoethyl)-2,3,4,5-tetrahydro-1H-benzo[e][1,4]diazepin-7-yl)-[1,1′-biphenyl]-3-yl)methoxy)-2-formylphenoxy)methyl)nicotinonitrile (0.5 g, 0.66 mmol) and 4 Å molecular sieve (0.1 g) in DCM (10 mL) was added (5S)-5-(aminomethyl)pyrrolidin-2-one (0.2 g, 1.4 mmol, as a hydrochloride salt) and TEA (0.5 mL, 3.5 mmol). The mixture was stirred at 25° C. for 12 h, then sodium triacetoxyborohydride (0.9 g, 4.2 mmol) was added to the mixture and stirred for 1 min. The reaction mixture was filtered and the filtrate was concentrated under reduced pressure to give a residue. The residue was purified by reverse phase HPLC to afford 5-((4-chloro-5-((2,2′-dimethyl-3′-(4-methyl-2,5-dioxo-1-(2-((((S)-5-oxopyrrolidin-2-yl)methyl)amino)ethyl)-2,3,4,5-tetrahydro-1H-benzo[e][1,4]diazepin-7-yl)-[1,1′-biphenyl]-3-yl)methoxy)-2-(((((S)-5-oxopyrrolidin-2-yl)methyl)amino)methyl)phenoxy)methyl)nicotinonitrile (165.85 mg, 12% yield, m/z: 909 [M+H]⁺ observed). ¹H NMR (400 MHz, CD₃OD): δ8.92 (m, 2H), 8.37 (m, 2H), 7.75 (s, 1H), 7.59 (m, 1H), 7.45 (m, 2H), 7.32 (t, J=8 Hz, 1H), 7.25 (m, 2H), 7.14 (m, 2H), 7.01 (m, 1H), 5.31 (m, 4H), 4.37 (br, 1H), 4.21 (m, 1H), 4.08 (m, 2H), 3.93 (br, 2H), 3.75 (m, 1H), 3.25 (m, 3H), 2.97 (br, 3H), 2.71 (m, 1H), 2.30 (m, 4H), 2.13 (s, 3H), 1.90 (s, 3H), 1.78 (br, 2H).

### Example 21: (Z)-8-(3′-(2-Fluoro-2-(5-(((2-hydroxyethyl)amino)methyl)pyridin-2-yl)vinyl)-2,2′-dimethyl-[1,1′-biphenyl]-3-yl)-3-(((2-hydroxyethyl)amino)methyl)-4H-pyrido[1,2-a]pyrimidin-4-one

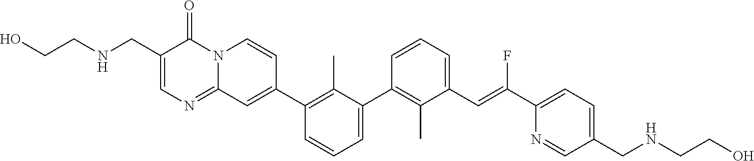 [SMILES: Cc1c(/C=C(\F)c2ccc(CNCCO)cn2)cccc1-c1cccc(-c2ccn3c(=O)c(CNCCO)cnc3c2)c1C]

### 2-Bromo-5-(1,3-dioxolan-2-yl)pyridine

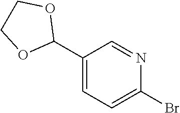 [SMILES: Brc1ccc(C2OCCO2)cn1]

To a mixture 6-bromonicotinaldehyde (50 g, 269 mmol) in toluene (350 mL) was added ethane-1,2-diol (50.1 g, 806 mmol) and 4-methylbenzenesulfonic acid (4.63 g, 26.9 mmol). After that, the mixture was stirred at 130° C. for 2.5 hr. The reaction mixture was combined with another batch at same scale. The reaction mixture was concentrated under reduced pressure to give a residue, and then poured into water (600 mL) and extracted with EtOAc (3×400 mL). The combined organic layers were washed with saturated aqueous brine solution (400 mL), dried over anhydrous sodium sulfate, filtered and concentrated under reduced pressure to give a residue. The residue was purified by normal phase SiO₂ chromatography (0-100% EtOAc/petroleum ether) to give 2-bromo-5-(1,3-dioxolan-2-yl)pyridine as yellow oil (106 g, 86% yield). ¹H NMR (400 MHz, CDCl₃): δ 8.43 (d, J=2.4 Hz, 1H), 7.62 (dd, J=2.4 Hz, J=8.4 Hz, 1H), 7.47 (d, J=8.4 Hz, 1H), 5.19 (s, 1H), 4.20-3.89 (m, 4H)

### Ethyl 2-(5-(1,3-dioxolan-2-yl)pyridin-2-yl)-2,2-difluoroacetate

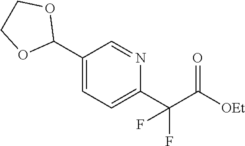 [SMILES: CCOC(=O)C(F)(F)c1ccc(C2OCCO2)cn1]

To a mixture 2-bromo-5-(1,3-dioxolan-2-yl)pyridine (52 g, 226 mmol) in DMSO (250 mL) was added ethyl 2-bromo-2,2-difluoroacetate (91.8 g, 452 mmol) and Cu (57.5 g, 904 mmol). After that, the mixture was stirred at r.t. for 48 hr. The reaction mixture was combined with another batch at same scale. The reaction mixture was filtered and the filtrate was partitioned between EtOAc (1000 mL) and saturated aqueous ammonium chloride solution (800 mL). The organic phase was separated, washed with EtOAc (3×500 mL), dried over anhydrous sodium sulfate, filtered and concentrated under reduced pressure to give a residue. The residue was purified by normal phase SiO₂ chromatography (0-100% EtOAc/petroleum ether) to afford ethyl 2-(5-(1,3-dioxolan-2-yl)pyridin-2-yl)-2,2-difluoroacetate as yellow oil (80 g, 65% yield). ¹H NMR (400 MHz, CDCl₃): δ 8.72 (s, 1H), 7.95 (dd, J=2 Hz, J=8 Hz, 1H), 7.74 (d, J=8 Hz, 1H), 5.89 (s, 1H), 4.35 (q, J=7.2 Hz, 2H), 4.18-4.0 (m, 4H), 1.31 (t, J=7.6 Hz, 3H)

### 2-(5-(1,3-Dioxolan-2-yl)pyridin-2-yl)-2,2-difluoroethanol

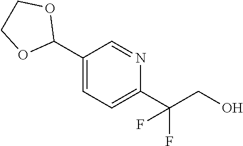 [SMILES: OCC(F)(F)c1ccc(C2OCCO2)cn1]

To a mixture ethyl 2-(5-(1,3-dioxolan-2-yl)pyridin-2-yl)-2,2-difluoroacetate (50 g, 183 mmol) in THE (800 mL) was added sodium borohydride (10.4 g, 274 mmol) at −20° C. for 1 hr. Then MeOH (400 mL) was added at −20° C. and the mixture was stirred at −20° C. for 2 hr. The reaction mixture was combined with another batch at same scale and concentrated under reduced pressure. The mixture was quenched by addition water (1500 ml) at 0° C., and then extracted with EtOAc (3×800 mL). The combined organic solution was dried over anhydrous sodium sulfate, filtered and concentrated under reduced pressure to give a residue. The residue was purified by normal phase SiO₂ chromatography (0-100% EtOAc/petroleum ether) to afford 2-(5-(1,3-dioxolan-2-yl)pyridin-2-yl)-2,2-difluoroethanol as yellow oil (47 g, 56% yield). ¹H NMR (400 MHz, CDCl₃): δ 8.70 (s, 1H), 7.96 (dd, J=2 Hz, J=8 Hz, 1H), 7.73 (d, J=8 Hz, 1H), 5.91 (s, 1H), 4.23 (t, J=1.28 Hz, 2H), 4.15-4.05 (m, 4H)

### 2-(5-(1,3-Dioxolan-2-yl)pyridin-2-yl)-2,2-difluoroethyl methanesulfonate

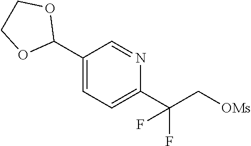 [SMILES: CS(=O)(=O)OCC(F)(F)c1ccc(C2OCCO2)cn1]

To a mixture 2-(5-(1,3-dioxolan-2-yl)pyridin-2-yl)-2,2-difluoroethanol (47 g, 203 mmol) in DCM (500 mL) was added TEA (62 mL, 447 mmol) and methylsulfamoyl chloride (46.6 g, 407 mmol) at 0° C. for 0.5 h. After that, the mixture was stirred at r.t. for 0.5 h. The reaction mixture was poured into ice water (1000 mL) and extracted with EtOAc (3×600 mL). The combined organic layers were washed with saturated aqueous brine solution (800 mL), dried over anhydrous sodium sulfate, filtered and concentrated under reduced pressure to give 2-(5-(1,3-dioxolan-2-yl)pyridin-2-yl)-2,2-difluoroethyl methanesulfonate as yellow oil (69 g, crude). ¹H NMR (400 MHz, CDCl₃): δ 8.73 (s, 1H), 7.95 (dd, J=2 Hz, J=8.4 Hz, 1H), 7.72 (d, J=8 Hz, 1H), 5.89 (s, 1H), 4.86 (t, J=12.8 Hz, 2H), 4.16-4.05 (m, 4H), 3.04 (s, 3H).

### 2-(1,1-Difluoro-2-iodoethyl)-5-(1,3-dioxolan-2-yl)pyridine

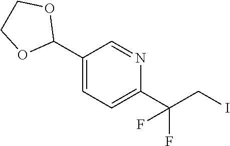 [SMILES: FC(F)(CI)c1ccc(C2OCCO2)cn1]

To a mixture 2-(5-(1,3-dioxolan-2-yl)pyridin-2-yl)-2,2-difluoroethyl methanesulfonate (69 g, 223 mmol) in DMA (54 mL) was added sodium iodide (134 g, 892 mmol). After that, the 20 mixture was stirred at 110° C. for 36 hr. The reaction mixture was poured into water (50 mL) and extracted with EtOAc (3×100 mL). The combined organic layers were washed with saturated aqueous brine solution (50 mL), dried over anhydrous sodium sulfate, filtered and concentrated under reduced pressure to give a residue. The residue was purified by normal phase SiO₂ chromatography (0-100% EtOAc/petroleum ether) to afford 2-(1,1-difluoro-2-iodoethyl)-5-(1,3-dioxolan-2-yl)pyridine (3 g, 4% yield) as a yellow oil and 6-(1,1-difluoro-2-iodoethyl)nicotinaldehyde (22 g, 33% yield) as a white solid. To a mixture 6-(1,1-difluoro-2-iodoethyl)nicotinaldehyde (22 g, 74.1 mmol) in toluene (120 mL) was added ethane-1,2-diol (18.4 g, 296 mmol) and 4-methylbenzenesulfonic acid (1.28 g, 7.41 mmol). After that, the mixture was stirred at 130° C. for 2.5 hr. The reaction mixture was added saturated sodium bicarbonate aqueous solution (500 mL), and extracted with EtOAc (3×200 mL), dried over anhydrous sodium sulfate, filtered and concentrated under reduced pressure to give a residue. The residue was purified normal phase SiO₂ chromatography (0-100% EtOAc/petroleum ether) to afford 2-(1,1-difluoro-2-iodoethyl)-5-(1,3-dioxolan-2-yl)pyridine as a yellow oil (20 g, 79% yield). ¹H NMR (400 MHz, CDCl₃): δ 8.67 (s, 1H), 7.95 (dd, J=2 Hz, J=8.4 Hz, 1H), 7.62 (d, J=8 Hz, 1H), 5.84 (s, 1H), 4.10-4.00 (m, 4H), 3.86 (t, J=6.4 Hz, 2H)

### (Z)-5-(1,3-Dioxolan-2-yl)-2-(1-fluoro-2-iodovinyl)pyridine

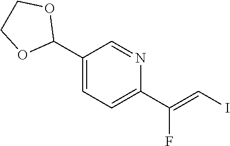 [SMILES: F/C(=C\I)c1ccc(C2OCCO2)cn1]

To a mixture 2-(1,1-difluoro-2-iodoethyl)-5-(1,3-dioxolan-2-yl)pyridine (20 g, 58.6 mmol) in DMSO (35 mL) was added potassium hydroxide (19.7 g, 351 mmol) in water (15 mL) at 0° C. for 0.5 h. After that, the mixture was stirred at r.t. for 11.5 h. The reaction mixture was poured into water (10 mL) and filtered to give (Z)-5-(1,3-dioxolan-2-yl)-2-(1-fluoro-2-iodovinyl)pyridine as white solid (15 g, 80% yield). ¹H NMR (400 MHz, CDCl₃): δ 8.66 (s, 1H), 7.85 (dd, J=1.6 Hz, J=8 Hz, 1H), 7.55 (d, J=8 Hz, 1H), 6.97 (d, J=34.4 Hz, 1H), 5.88 (s, 1H), 4.14-4.08 (m, 4H).

### (Z)-2-(2-(3-Bromo-2-methylphenyl)-1-fluorovinyl)-5-(1,3-dioxolan-2-yl)pyridine

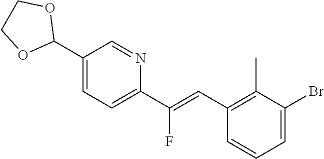 [SMILES: Cc1c(Br)cccc1/C=C(\F)c1ccc(C2OCCO2)cn1]

To a mixture (Z)-5-(1,3-dioxolan-2-yl)-2-(1-fluoro-2-iodovinyl)pyridine (5 g, 15.6 mmol,) in dioxane/H₂O mixture (3:1, 40 mL) was added (3-bromo-2-methylphenyl)boronic acid (3.35 g, 15.6 mmol), potassium phosphate (6.61 g, 31.1 mmol) and chloro[(di(1-adamantyl)-N-butylphosphine)-2-(2-aminobiphenyl)]palladium(II) (1.04 g, 1.56 mmol). After that, the mixture was stirred at 70° C. for 1 hr. The reaction mixture was concentrated under reduced pressure to give a residue, and then poured into water (60 mL) and extracted with EtOAc (3×40 mL). The combined organic layers were washed with saturated aqueous brine solution (50 mL), dried over anhydrous sodium sulfate, filtered and concentrated under reduced pressure to give a residue. The residue was purified by normal phase SiO₂ chromatography (0-100% EtOAc/petroleum ether) to afford (Z)-2-(2-(3-bromo-2-methylphenyl)-1-fluorovinyl)-5-(1,3-dioxolan-2-yl)pyridine as a yellow oil.((5 g, 88% yield)¹H NMR (400 MHz, CDCl₃): δ 8.64 (s, 1H), 7.79 (dd, J=2 Hz, J=8 Hz, 1H), 7.62 (d, J=8 Hz, 1H), 7.56 (dd, J=1.6 Hz, J=8.4 Hz, 1H), 7.43 (d, J=8 Hz, 1H), 7.25 (d, J=37.2 Hz, 1H), 7.01 (t, J=8 Hz, 1H), 5.83 (s, 1H), 4.07-4.00 (m, 4H), 2.45 (s, 3H).

### (Z)-6-(2-(3-Bromo-2-methylphenyl)-1-fluorovinyl)nicotinaldehyde

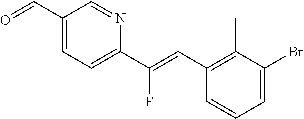 [SMILES: Cc1c(Br)cccc1/C=C(\F)c1ccc(C=O)cn1]

To a mixture of (Z)-2-(2-(3-bromo-2-methylphenyl)-1-fluorovinyl)-5-(1,3-dioxolan-2-yl)pyridine (0.45 g, 1.24 mmol) in THE (5 mL) was added concentrated HCl (3 mL) in one portion. The mixture was stirred at r.t. for 2 h. The mixture was neutralized with saturated aqueous sodium carbonate solution. The mixture was extracted with ethyl acetate (2×30 mL). The combined organic phase was washed with saturated aqueous brine solution (2×20 mL), dried with anhydrous sodium sulfate, filtered and concentrated to give the residue. The residue was purified by normal phase SiO₂ chromatography (0-30% ethyl acetate/petroleum ether) to afford (Z)-6-(2-(3-bromo-2-methylphenyl)-1-fluorovinyl)nicotinaldehyde as a yellow solid (0.32 g, 81% yield). ¹H NMR (400 MHz, CDCl₃): δ 10.13 (s, 1H), 9.06 (s, 1H), 8.24 (dd, J₁=8.4 Hz, J₂=2 Hz, 1H), 7.77 (d, J=8 Hz, 1H), 7.73 (d, J=8 Hz, 1H), 7.56 (s, 0.5H), 7.54 (d, J=8 Hz, 1H), 7.47 (s, 0.5H), 7.10 (t, J=8 Hz, 1H), 2.54 (s, 3H).

### (Z)-tert-Butyl ((6-(2-(3-bromo-2-methylphenyl)-1-fluorovinyl)pyridin-3-yl)methyl)(2-hydroxyethyl)carbamate

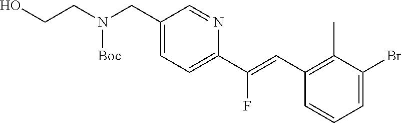 [SMILES: Cc1c(Br)cccc1/C=C(\F)c1ccc(CN(CCO)C(=O)OC(C)(C)C)cn1]

To a mixture of (Z)-6-(2-(3-bromo-2-methylphenyl)-1-fluorovinyl)nicotinaldehyde (0.32 g, 1 mmol) and 2-aminoethanol (0.18 g, 3 mmol) in THF/MeOH mixture (6 mL, 2:1) was added glacial AcOH (0.09 g, 1.5 mmol) in one portion under N₂. The mixture was stirred at r.t. for 12 h. To the mixture was added sodium cyanoborohydride (0.25 g, 4 mmol). The mixture was stirred at r.t. for 0.5 h. To the mixture was added di-tert-butyl dicarbonate (1.53 g, 7 mmol) and triethylamine (0.56 mL, 4 mmol) in one portion under N₂. The mixture was stirred at r.t. for 1 h. The mixture was concentrated to give the residue. To the residue was added water (50 mL). The mixture was extracted with ethyl acetate (2×100 mL). The combined organic phase was washed with saturated aqueous brine solution (2×40 mL), dried with anhydrous sodium sulfate, filtered and concentrated to give the residue. The residue was purified by normal phase SiO₂ chromatography (0-30% ethyl acetate/petroleum ether) to afford (Z)-tert-butyl ((6-(2-(3-bromo-2-methylphenyl)-1-fluorovinyl)pyridin-3-yl)methyl)(2-hydroxyethyl)carbamate as a yellow oil (0.52 g, 61% yield). m/z: 465 [M]⁺, 467 [M+2]⁺ observed.

### tert-Butyl N-[[6-[(Z)-2-[3-[3-[3-[[tert-butoxycarbonyl(2-hydroxyethyl)amino]methyl]-4-oxo-pyrido[1,2-a]pyrimidin-8-yl]-2-methyl-phenyl]-2-methyl-phenyl]-1-fluoro-vinyl]-3-pyridyl]methyl]-N-(2-hydroxyethyl)carbamate

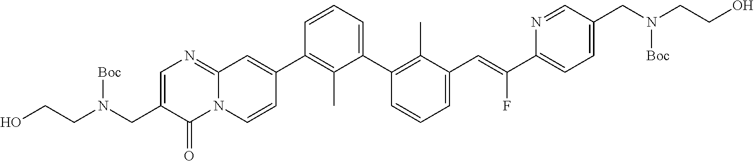 [SMILES: Cc1c(/C=C(\F)c2ccc(CN(CCO)C(=O)OC(C)(C)C)cn2)cccc1-c1cccc(-c2ccn3c(=O)c(CN(CCO)C(=O)OC(C)(C)C)cnc3c2)c1C]

To a mixture of (Z)-tert-butyl ((6-(2-(3-bromo-2-methylphenyl)-1-fluorovinyl)pyridin-3-yl)methyl)(2-hydroxyethyl)carbamate (142 mg, 0.17 mmol, 55% purity) and tert-butyl (2-hydroxyethyl)((8-(2-methyl-3-(4,4,5,5-tetramethyl-1,3,2-dioxaborolan-2-yl)phenyl)-4-oxo-4H-pyrido[1,2-a]pyrimidin-3-yl)methyl)carbamate (90 mg, 0.17 mmol) in THF/H₂O mixture (5 ml, 5:1) was added potassium carbonate (93 mg, 0.67 mmol) and Pd(dppf)Cl₂.CH₂Cl₂ (14 mg, 0.02 mmol) in one portion under N₂. The mixture was stirred at 80° C. for 1 h. The mixture was combined with another batch at same scale. The mixture was concentrated to give the residue. Water (20 mL) was added and the mixture was extracted with ethyl acetate (2×20 mL). The combined organic phase was washed with saturated aqueous brine solution (20 mL), dried with anhydrous sodium sulfate, filtered and concentrated to give the residue. The residue was purified by normal phase SiO₂ chromatography (0-90% 30% THF in ethyl acetate/petroleum ether) to afford tert-butyl N-[[6-[(Z)-2-[3-[3-[3-[[tert-butoxycarbonyl(2-hydroxyethyl)amino]methyl]-4-oxo-pyrido[1,2-a]pyrimidin-8-yl]-2-methyl-phenyl]-2-methyl-phenyl]-1-fluoro-vinyl]-3-pyridyl]methyl]-N-(2-hydroxyethyl)carbamate as a yellow gum (160 mg, 37% yield, 65% purity, m/z: 794 [M+H]⁺ observed).

### (Z)-8-(3′-(2-fluoro-2-(5-(((2-hydroxyethyl)amino)methyl)pyridin-2-yl)vinyl)-2,2′-dimethyl-[1,1′-biphenyl]-3-yl)-3-(((2-hydroxyethyl)amino)methyl)-4H-pyrido[1,2-a]pyrimidin-4-one

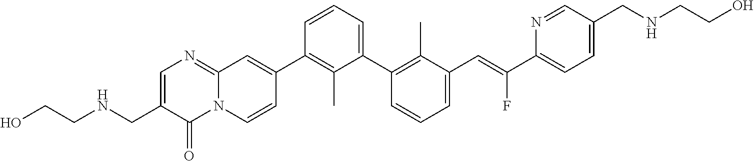 [SMILES: Cc1c(/C=C(\F)c2ccc(CNCCO)cn2)cccc1-c1cccc(-c2ccn3c(=O)c(CNCCO)cnc3c2)c1C]

To a mixture of tert-butyl N-[[6-[(Z)-2-[3-[3-[3-[[tert-butoxycarbonyl(2-hydroxyethyl)amino]methyl]-4-oxo-pyrido[1,2-a]pyrimidin-8-yl]-2-methyl-phenyl]-2-methyl-phenyl]-1-fluoro-vinyl]-3-pyridyl]methyl]-N-(2-hydroxyethyl)carbamate (150 mg, 0.12 mmol, 65% purity) in MeOH (2 mL) was added HCl (4 M solution in 1,4-dioxane, 2 mL) in one portion under N₂. The mixture was stirred at r.t. for 1 h. The residue was purified by reverse phase HPLC to afford (Z)-8-(3′-(2-fluoro-2-(5-(((2-hydroxyethyl)amino)methyl)pyridin-2-yl)vinyl)-2,2′-dimethyl-[1,1′-biphenyl]-3-yl)-3-(((2-hydroxyethyl)amino)methyl)-4H-pyrido[1,2-a]pyrimidin-4-one as a white solid (52.93 mg, 70% yield, 97% purity, m/z: 594 [M+H]⁺ observed). ¹H NMR (400 MHz, CD₃OD): δ9.14 (d, J=7.6 Hz, 1H), 8.62 (s, 1H), 8.42 (s, 1H), 7.94 (dd, J₁=8 Hz, J₂=2 Hz, 1H), 7.84 (d, J=8 Hz, 1H), 7.73 (d, J=7.6 Hz, 1H), 7.67 (d, J=1.2 Hz, 1H), 7.48-7.43 (m, 3H), 7.38 (s, 0.5H), 7.36-7.33 (m, 1H), 7.30-7.28 (m, 1.5H), 7.14 (d, J=6.8 Hz, 1H), 3.89 (d, J=6.4 Hz, 4H), 3.73-3.70 (m, 4H), 2.81-2.76 (m, 4H), 2.19 (s, 3H), 2.07 (s, 3H).

### Example 22: 5-((4-Chloro-5-((3′-(5-((2-hydroxyethyl)amino)-1-methyl-2-oxo-2,3,4,5-tetrahydro-1H-benzo[b]azepin-8-yl)-2,2′-dimethyl-[1,1′-biphenyl]-3-yl)methoxy)-2-(((2-hydroxyethyl)amino)methyl)phenoxy)methyl)nicotinonitrile

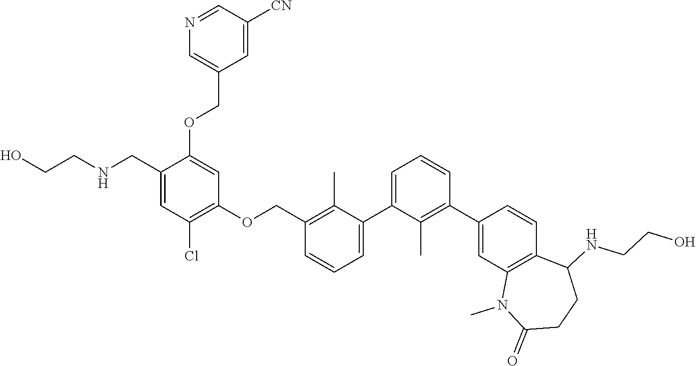 [SMILES: Cc1c(COc2cc(OCc3cncc(C#N)c3)c(CNCCO)cc2Cl)cccc1-c1cccc(-c2ccc3c(c2)N(C)C(=O)CCC3NCCO)c1C]

### 8-Bromo-1,3,4,5-tetrahydro-2H-benzo[b]azepin-2-one

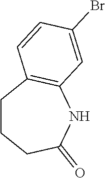 [SMILES: O=C1CCCc2ccc(Br)cc2N1]

To a stirring solution of 7-bromotetralin-1-one, (11.30 g, 50.2 mmol, 1 eq) in 100 mL of dry toluene at rt under nitrogen in a two necked round bottom flask, equipped with an addition funnel, was added sodium azide (13.06 g, 200.8 mmol, 4 eq) in one portion. The solution was cooled to 0° C. in and concentrated sulfuric acid, (12.55 mL, 150.6 mmol, 3 eq) was added dropwise over 0.5 h via an addition funnel. The reaction was warmed to rt and stirred for 16 h. The reaction was slowly quenched by the addition of sat aq NaHCO₃ solution, allowing off gassing to occur and avoid overflow. The organic phase was separated, and the aqueous phase was extracted with ethyl acetate (100 mL×3). Combined organic layers were washed with sat aq NaHCO₃ solution (75 mL×2), dried over anhydrous sodium sulfate, filtered, and concentrated under reduced pressure. The dark brown solid/sludge was suspended with 25 mL of diethyl ether, stirred for 15 minutes, filtered, and rinsed with an additional 25 mL of diethyl ether. The resulting light tan was dried and used without further purification as 8-bromo-1,3,4,5-tetrahydro-2H-benzo[b]azepin-2-one (9.0 g, 75% yield, m/z: 240 [M+H]⁺ observed). ¹H NMR (400 MHz, CDCl₃): δ 8.11 (s, 1H), 7.26 (dd, J=8.0, 2.0 Hz, 1H), 7.16 (d, J=2.0 Hz, 1H), 7.09 (d, J=8.1 Hz, 1H), 2.76 (t, J=7.2 Hz, 2H), 2.36 (tt, J=7.3, 1.0 Hz, 2H), 2.23 (pd, J=7.3, 1.2 Hz, 2H).

### 8-Bromo-1-methyl-1,3,4,5-tetrahydro-2H-benzo[b]azepin-2-one

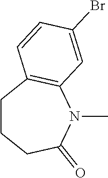

To a stirring solution of 8-bromo-1,3,4,5-tetrahydro-1-benzazepin-2-one (5.00 g, 20.82 mmol, 1 eq) in dry DMF (40 mL) at rt was added cesium carbonate (13.57 g, 41.65 mmol, 2 eq) in one portion, followed by neat iodomethane (1.56 mL, 24.99 mmol, 1.2 eq) dropwise via syringe. The white suspension was stirred for 1.5 h at rt. The reaction was diluted with diethyl ether (50 mL) and quenched by the addition of water (100 mL). The biphasic solution was vigorously agitated and then let settle. The aqueous phase was extracted with diethyl ether (50 mL×2), combined organic layers were washed with water (50 mL×2), dried over anhydrous sodium sulfate, filtered, and concentrated under vacuum. The crude residue was purified by normal phase SiO₂ chromatography (0-30% EA/hexanes) to afford 8-bromo-1-methyl-1,3,4,5-tetrahydro-2H-benzo[b]azepin-2-one (5.29 g, 100% yield, m/z: 254 [M+H]⁺ observed). ¹H NMR (400 MHz, CDCl₃): δ 7.34-7.24 (m, 2H), 7.06 (d, J=8.0 Hz, 1H), 3.33 (s, 3H), 2.67 (t, J=6.9 Hz, 2H), 2.29 (t, J=6.9 Hz, 2H), 2.20-2.13 (m, 2H).

### 5,8-Dibromo-1-methyl-1,3,4,5-tetrahydro-2H-benzo[b]azepin-2-one

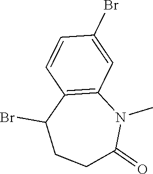

To a stirring solution of 8-bromo-1-methyl-4,5-dihydro-3H-1-benzazepin-2-one (5.30 g, 20.86 mmol, 1 eq) in dry DCM (40 mL) at rt was added benzoyl benzenecarboperoxoate (1.52 g, 6.26 mmol, 0.3 eq), followed by 1,3-dibromo-5,5-dimethyl-imidazolidine-2,4-dione (8.94 g, 31.28 mmol, 1.5 eq) each in one portion. The reaction was heated to 40° C. for 14 h with vigorous stirring. The reaction was cooled to rt, diluted with DCM (20 mL) and quenched by the addition of water (40 mL). The aqueous phase was extracted with DCM (2×20 mL). Combined organic phases were dried over anhydrous sodium sulfate, filtered, and concentrated to a crude orange/red oil. The residue was purified by normal phase SiO₂ chromatography (0-30% EA/hexanes) to afford 5,8-dibromo-1-methyl-1,3,4,5-tetrahydro-2H-benzo[b]azepin-2-one (2.95 g, 42% yield, m/z: 334 [M+2H]⁺ observed). ¹H NMR (400 MHz, CDCl₃): δ 7.37 (d, J=1.8 Hz, 1H), 7.34-7.31 (m, 1H), 5.21 (t, J=6 Hz, 1H), 3.36 (s, 3H), 2.86-2.58 (m, 2H), 2.37 (td, J=7.1, 2.1 Hz, 2H).

### 8-Bromo-5-((2-hydroxyethyl)amino)-1-methyl-1,3,4,5-tetrahydro-2H-benzo[b]azepin-2-one

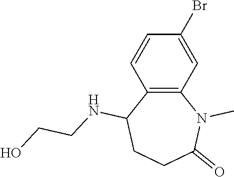 [SMILES: CN1C(=O)CCC(NCCO)c2ccc(Br)cc21]

To a stirring solution of 5,8-dibromo-1-methyl-4,5-dihydro-3H-1-benzazepin-2-one (1.05 g, 3.15 mmol, 1 eq) in dry DCE (12 mL) at rt was added 2-aminoethanol (0.95 mL,15.76 mmol, 5 eq) dropwise via syringe. The orange reaction was stirred at rt for 48 h, filtered through CELITE®, rinsed with DCM (20 mL), and concentrated under reduced pressure. The crude oil was purified by normal phase SiO₂ chromatography (0-10% MeOH/DCM) to afford (257 mg, 26% yield, m/z: 315 [M+2H]⁺ observed).

### tert-Butyl (8-bromo-1-methyl-2-oxo-2,3,4,5-tetrahydro-1H-benzo[b]azepin-5-yl)(2-((tert-butoxycarbonyl)oxy)ethyl)carbamate

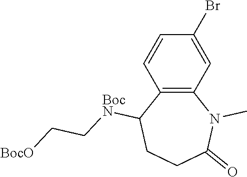

To a stirring mixture of 8-bromo-5-(2-hydroxyethylamino)-1-methyl-4,5-dihydro-3H-1-benzazepin-2-one (257.00 mg, 0.82 mmol, 1 eq) in DCM (4.8 mL) at rt was added triethylamine (456 uL, 3.28 mmol, 4 eq) dropwise via syringe and tert-butoxycarbonyl tert-butyl carbonate (358.18 mg, 1.64 mmol, 2 eq) in one portion. The yellow reaction heated to 45° C. for 18 h. The reaction mixture was cooled to rt, the solvent was removed under vacuum, and the crude dark orange oil was purified by normal phase SiO₂ chromatography (0-100% EA/hexanes) to afford (133 mg, 39% yield, m/z: 513 [M+H]⁺ observed).

### tert-Butyl (8-(3-bromo-2-methylphenyl)-1-methyl-2-oxo-2,3,4,5-tetrahydro-1H-benzo[b]azepin-5-yl)(2-((tert-butoxycarbonyl)oxy)ethyl)carbamate

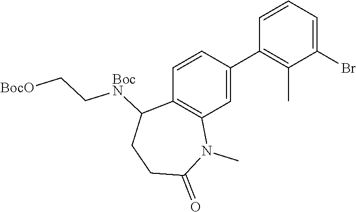

To a mixture of 2-[(8-bromo-1-methyl-2-oxo-4,5-dihydro-3H-1-benzazepin-5-yl)-tert-butoxycarbonyl-amino]ethyl tert-butyl carbonate (133.00 mg, 0.26 mmol, 1 eq) and (3-bromo-2-methyl-phenyl)boronic acid, (66.8 mg, 0.31 mmol, 1.2 eq) in THF/DMF/H₂O (2:2:1, 5 mL) was added tetrakis(triphenylphosphine)palladium (0) (29.9 mg, 0.03 mmol, 0.1 eq) and sodium carbonate (35.69 mg, 0.34 mmol, 1.3 eq) in one portion under N₂. The reaction was heated to 80° C. for 16 h and cooled to rt. The reaction was quenched by the addition of water (5 mL) and diluted with ethyl acetate (5 mL). The aqueous layer was extracted with ethyl acetate (10 mL×2). The combined organic phases were washed with water (10 mL×2), dried over anhydrous sodium sulfate, filtered, and concentrated. The crude mixture was purified by normal phase SiO₂ chromatography (0-100% EA/hexanes) to afford an inseparable mixture (60 mg, m/z: 605 [M+2H]⁺ observed). that was used in the following reaction.

### 5-((4-Chloro-5-((3′-(5-((2-hydroxyethyl)amino)-1-methyl-2-oxo-2,3,4,5-tetrahydro-1H-benzo[b]azepin-8-yl)-2,2′-dimethyl-[1,1′-biphenyl]-3-yl)methoxy)-2-(((2-hydroxyethyl)amino)methyl)phenoxy)methyl)nicotinonitrile

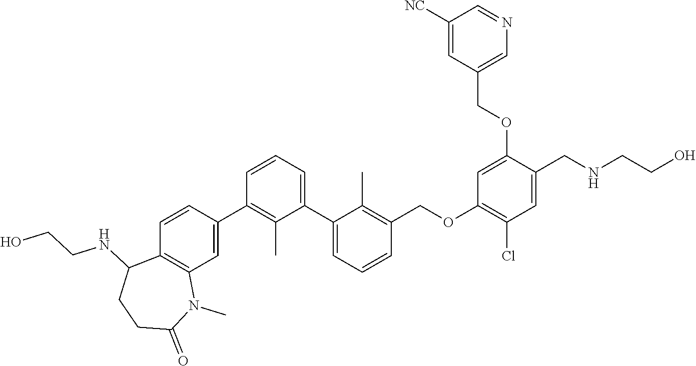 [SMILES: Cc1c(COc2cc(OCc3cncc(C#N)c3)c(CNCCO)cc2Cl)cccc1-c1cccc(-c2ccc3c(c2)N(C)C(=O)CCC3NCCO)c1C]

To a mixture of tert-butyl N-[8-(3-bromo-2-methyl-phenyl)-1-methyl-2-oxo-4,5-dihydro-3H-1-benzazepin-5-yl]-N-(2-hydroxyethyl)carbamate (60.00 mg, 0.10 mmol, 1 eq) tert-butyl N-[[5-chloro-2-[(5-cyano-3-pyridyl)methoxy]-4-[[2-methyl-3-(4,4,5,5-tetramethyl-1,3,2-dioxaborolan-2-yl)phenyl]methoxy]phenyl]methyl]-N-(2-hydroxyethyl)carbamate (70.34 mg, 0.10 mmol, 1 eq), and cesium carbonate (77.66 mg, 0.24 mmol, 2.5 eq) at rt was added a 3:1 mixture of dioxane/H₂O (4 mL). The reaction vessel was evacuated and backfilled with nitrogen (x3). Pd(dppf)Cl₂ (6.98 mg, 0.01 mmol, 0.1 eq) was added and the evacuation/backfilled process was repeated. After 3 h of stirring at 80° C., the reaction was cooled to rt and concentrated under reduced pressure. The residue was extracted with ethyl acetate (10 mL×2), combined organic layers were washed with brine (10 mL×2), dried over anhydrous sodium sulfate, filtered, and concentrated under reduced pressure. The crude mixture was purified by flash column chromatography 0-10% MeOH/DCM to afford a mixture products (45 mg, m/z: 870, 960 [M+H]⁺ observed). The crude material (45 mg) was dissolved in MeOH (2 mL) and treated with 4N HCl in dioxane (2 mL) at rt. The reaction mixture was stirred for 2.5 h and the solvents were removed under vacuum. The crude mixture was purified by HPLC C18 reverse phase from 0-80% MeCN/H₂O to afford 5-((4-chloro-5-((3′-(5-((2-hydroxyethyl)amino)-1-methyl-2-oxo-2,3,4,5-tetrahydro-1H-benzo[b]azepin-8-yl)-2,2′-dimethyl-[1,1′-biphenyl]-3-yl)methoxy)-2-(((2-hydroxyethyl)amino)methyl)phenoxy)methyl)nicotinonitrile (16.7 mg). m/z: 760 [M]⁺ observed. ¹H NMR (400 MHz, CDCl₃): δ 8.95 (dd, J=11.4, 2.1 Hz, 2H), 8.40 (t, J=2.1 Hz, 1H), 7.51 (s, 1H), 7.47 (d, J=7.2 Hz, 1H), 7.34-7.23 (m, 3H), 7.18-7.07 (m, 4H), 7.03 (s, 1H), 6.95 (s, 1H), 5.38 (s, 2H), 5.33 (s, 2H), 5.19 (s, 1H), 4.24 (s, 2H), 3.86-3.75 (m, 3H), 3.75-3.58 (m, 2H), 3.16-3.08 (m, 2H), 2.97 (s, 3H), 2.80 (ddd, J=13.0, 7.5, 4.7 Hz, 1H), 2.56 (q, J=7.4, 6.5 Hz, 3H), 2.15 (s, 3H), 1.99 (d, J=13.7 Hz, 1H), 1.91 (s, 3H).

### Example 23: 5-((4-Chloro-2-(((2-hydroxyethyl)amino)methyl)-5-((3′-(4-(((2-hydroxyethyl)amino)methyl)-2-oxopyridin-1(2H)-yl)-2,2′-dimethyl-[1,1′-biphenyl]-3-yl)methoxy)phenoxy)methyl)nicotinonitrile

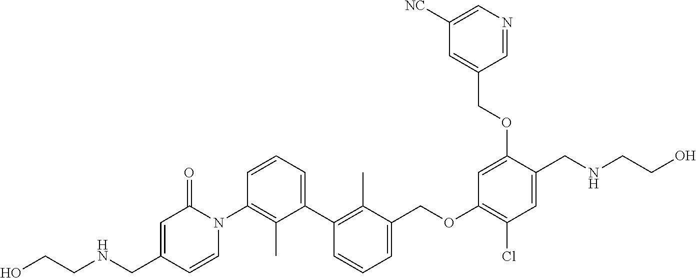 [SMILES: Cc1c(COc2cc(OCc3cncc(C#N)c3)c(CNCCO)cc2Cl)cccc1-c1cccc(-n2ccc(CNCCO)cc2=O)c1C]

### 1-(3-Bromo-2-methylphenyl)-2-oxo-1,2-dihydropyridine-4-carbaldehyde

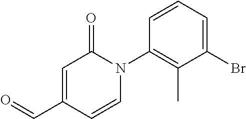 [SMILES: Cc1c(Br)cccc1-n1ccc(C=O)cc1=O]

To a solution of 2-hydroxyisonicotinaldehyde (5 g, 40.6 mmol) in dichloromethane (50 mL) and DMF (150 mL) was added (3-bromo-2-methylphenyl)boronic acid (11.3 g, 52.8 mmol), copper acetate (14.8 g, 81.2 mmol), pyridine (6.6 mL, 81.2 mmol) and 4 Å molecular sieve (1.5 g). The mixture was stirred at r.t. under oxygen gas (15 psi) for 12 hrs. The reaction mixture was filtered and the filtrate was concentrated under reduced pressure. The reaction mixture was quenched with water (200 mL) and extracted with ethyl acetate (3×30 mL). The combined organic layers were washed with saturated aqueous brine solution (3×15 mL), dried over anhydrous sodium sulfate, filtered and concentrated under reduced pressure. The residue was purified by normal phase flash SiO₂ chromatography (17˜33% ethyl acetate/petroleum ether) to afford 1-(3-bromo-2-methyl-phenyl)-2-oxo-pyridine-4-carbaldehyde (1.5 g, 13% yield) as a yellow solid.

### 1-(3-Bromo-2-methylphenyl)-4-(hydroxymethyl)pyridin-2(1H)-one

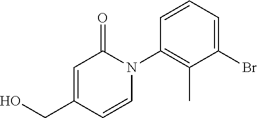 [SMILES: Cc1c(Br)cccc1-n1ccc(CO)cc1=O]

To a solution of 1-(3-bromo-2-methylphenyl)-2-oxo-1,2-dihydropyridine-4-carbaldehyde (600 mg, 2 mmol) in methanol (20 mL) was added sodium borohydride (100 mg, 2.6 mmol) in portions at 0° C. The mixture was stirred at r.t. under nitrogen gas for 20 min. The mixture was quenched with saturated aqueous ammonium chloride solution (5 mL). The mixture was diluted with THF (35 mL), washed with saturated aqueous brine solution (3×8 mL), dried over anhydrous sodium sulfate, filtered and concentrated under reduced pressure to give 1-(3-bromo-2-methylphenyl)-4-(hydroxymethyl)pyridin-2(1H)-one (626 mg, 86%) as a yellow solid. m/z: 294 [M]⁺, 296 [M+2]⁺ observed.

### 1-(3-Bromo-2-methylphenyl)-4-(chloromethyl)pyridin-2(1H)-one

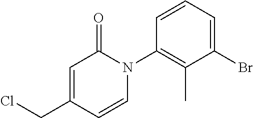 [SMILES: Cc1c(Br)cccc1-n1ccc(CCl)cc1=O]

To a solution of 1-(3-bromo-2-methylphenyl)-4-(hydroxymethyl)pyridin-2(1H)-one (626 mg, 2.1 mmol) in dichloromethane (20 mL) was added a solution of thionyl chloride (0.2 mL, 2.8 mmol) in dichloromethane (0.8 mL) at 0° C. The mixture was stirred at r.t. under nitrogen for 8 hrs. The reaction mixture as a yellow solution (21 mL) was used in the next step directly.

### 1-(3-Bromo-2-methylphenyl)-4-(((2-hydroxyethyl)amino)methyl)pyridin-2(1H)-one

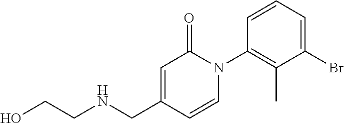 [SMILES: Cc1c(Br)cccc1-n1ccc(CNCCO)cc1=O]

To the above solution of 1-(3-bromo-2-methylphenyl)-4-(chloromethyl)pyridin-2(1H)-one in dichloromethane (21 mL) was added 2-aminoethanol (1.2 mL, 20 mmol), potassium carbonate (1.5 g, 10.8 mmol), potassium iodide (200 mg, 1.2 mmol), DMF (15 mL) and THE (14 mL). The mixture was stirred at 60° C. under nitrogen gas for 12 hrs. The reaction mixture as a white suspension (˜60 mL) was used in the next step directly.

### tert-Butyl ((1-(3-bromo-2-methylphenyl)-2-oxo-1,2-dihydropyridin-4-yl)methyl)(2-hydroxyethyl)carbamate

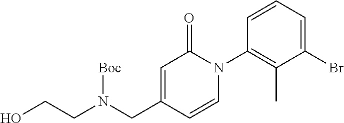 [SMILES: Cc1c(Br)cccc1-n1ccc(CN(CCO)C(=O)OC(C)(C)C)cc1=O]

To the above suspension of 1-(3-bromo-2-methylphenyl)-4-(((2-hydroxyethyl)amino)methyl) pyridin-2(1H)-one in a mixed solvents of dichloromethane (21 mL), THF (14 mL) and DMF (15 mL) was added di-tert-butyl dicarbonate (6 mL, 26 mmol). The mixture was stirred at r.t. under nitrogen for 12 hrs. The mixture was diluted with THE (20 mL), washed with saturated aqueous brine solution (4×10 mL), dried over anhydrous sodium sulfate, filtered and concentrated under reduced pressure. The residue was purified by normal phase flash SiO₂ chromatography (90-33% petroleum ether/50% THE in ethyl acetate) to afford tert-butyl ((1-(3-bromo-2-methylphenyl)-2-oxo-1,2-dihydropyridin-4-yl)methyl)(2-hydroxyethyl)carbamate (800 mg, 86% yield for three steps, m/z: 437 [M]⁺, 439 [M+2]⁺ observed) as a yellow gum.

### 5-((5-((3-Bromo-2-methylbenzyl)oxy)-4-chloro-2-(((2-hydroxyethyl)amino)methyl)phenoxy)methyl)nicotinonitrile

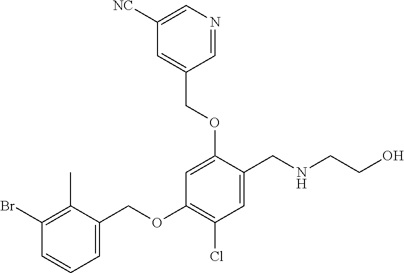 [SMILES: Cc1c(Br)cccc1COc1cc(OCc2cncc(C#N)c2)c(CNCCO)cc1Cl]

To a solution of 5-((5-((3-bromo-2-methylbenzyl)oxy)-4-chloro-2-formylphenoxy)methyl)nicotinonitrile (5 g, 10.6 mmol) and 2-aminoethanol (3 mL, 49.6 mmol) in THF (90 mL), DMF (50 mL) and 1,2-dichloroethane (30 mL) was added glacial acetic acid (0.5 mL, 8.7 mmol) and 4 Å molecular sieve (1 g). The mixture was stirred at r.t. under nitrogen gas for 12 hrs. Sodium cyanoborohydride (3.5 g, 55.7 mmol) was added and the mixture was stirred at r.t. for 12 hrs. The mixture was concentrated to about 50 mL. To the residue was added water (100 mL) and a lot of yellow solid was precipitated. The resulting yellow solid was collected by filtration, washed with water (2×10 mL), petroleum ether (20 mL) and dried under reduced pressure to give 5-((5-((3-bromo-2-methylbenzyl)oxy)-4-chloro-2-(((2-hydroxyethyl)amino)methyl)phenoxy)methyl)nicotinonitrile (6.5 g, crude, m/z: 516 [M]⁺, 518 [M+2]⁺ observed) which was used in the next step directly.

### tert-Butyl 4-((3-bromo-2-methylbenzyl)oxy)-5-chloro-2-((5-cyanopyridin-3-yl)methoxy)benzyl(2-hydroxyethyl)carbamate

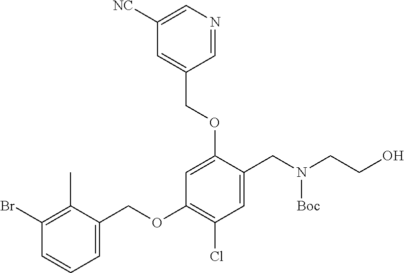 [SMILES: Cc1c(Br)cccc1COc1cc(OCc2cncc(C#N)c2)c(CN(CCO)C(=O)OC(C)(C)C)cc1Cl]

To a solution of 5-((5-((3-bromo-2-methylbenzyl)oxy)-4-chloro-2-(((2-hydroxyethyl)amino)methyl)phenoxy)methyl)nicotinonitrile (6.5 g, 12.6 mmol) in THE (100 mL) was added di-tert-butyl dicarbonate (12 mL, 52.2 mmol) and triethylamine (5 mL, 36 mmol). The mixture was stirred at r.t. for 12 hrs. The mixture was washed with saturated aqueous brine solution (4×40 mL), dried over anhydrous sodium sulfate, filtered and concentrated under reduced pressure. The residue was purified by normal phase flash SiO₂ chromatography (90-33% petroleum ether/50% THF in ethyl acetate) to give tert-butyl 4-((3-bromo-2-methylbenzyl)oxy)-5-chloro-2-((5-cyanopyridin-3-yl) methoxy)benzyl(2-hydroxyethyl)carbamate (2.7 g, 29% yield, m/z: 560 [M-tButyl]⁺, 562 [M-tButyl+2]⁺ observed) as a yellow solid. ¹H NMR (400 MHz, CDCl₃): δ 8.89 (s, 1H), 8.86 (s, 1H), 8.07 (br s, 1H), 7.58 (d, J=7.6 Hz, 1H), 7.37 (d, J=7.6 Hz, 1H), 7.26 (s, 1H), 7.09 (t, J=8.0 Hz, 1H), 6.52 (s, 1H), 5.11 (s, 2H), 5.08 (s, 2H), 4.47 (br s, 2H), 3.71 (br s, 2H), 3.39 (br s, 2H), 2.47 (s, 3H), 1.47 (s, 9H).

### tert-Butyl 5-chloro-2-((5-cyanopyridin-3-yl)methoxy)-4-((2-methyl-3-(4,4,5,5-tetramethyl-1,3,2-dioxaborolan-2-yl)benzyl)oxy)benzyl(2-hydroxyethyl)carbamate

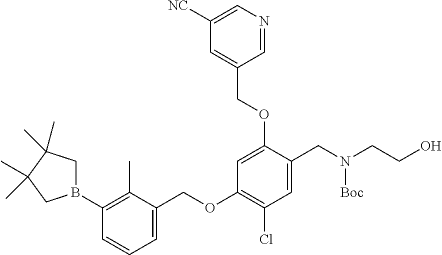 [SMILES: Cc1c(COc2cc(OCc3cncc(C#N)c3)c(CN(CCO)C(=O)OC(C)(C)C)cc2Cl)cccc1B1CC(C)(C)C(C)(C)C1]

To a solution of tert-butyl 4-((3-bromo-2-methylbenzyl)oxy)-5-chloro-2-((5-cyanopyridin-3-yl)methoxy)benzyl(2-hydroxyethyl)carbamate (1.2 g, 1.9 mmol) and 1,1′-bis(diphenylphosphino)ferrocene-palladium(II)dichloride dichloromethane complex (240 mg, 0.29 mmol) in THE (20 mL) and dioxane (20 mL) was added bis (pinacolato)diboron (1.8 g, 7.1 mmol) and potassium acetate (600 mg, 6.1 mmol). The mixture was stirred at 90° C. under nitrogen gas for 15 hrs. The mixture was diluted with THE (20 mL), washed with saturated aqueous brine solution (2×10 mL), dried over anhydrous sodium sulfate, filtered and concentrated under reduced pressure. The residue was purified by normal phase flash SiO₂ chromatography (90-50% petroleum ether/50% THE in ethyl acetate) to afford tert-butyl 5-chloro-2-((5-cyanopyridin-3-yl)methoxy)-4-((2-methyl-3-(4,4,5,5-tetramethyl-1,3,2-dioxaborolan-2-yl)benzyl)oxy)benzyl(2-hydroxyethyl)carbamate (910 mg, 56% yield, m/z: 608 [M-tButyl+H]⁺ observed) as a yellow solid. ¹H NMR (400 MHz, CDCl₃): δ 8.88 (s, 1H), 8.84 (d, J=2.0 Hz, 1H), 8.04 (br s, 1H), 7.76 (d, J=7.2 Hz, 1H), 7.48 (d, J=7.6 Hz, 1H), 7.25 (s, 1H), 7.23-7.19 (m, 1H), 6.50 (s, 1H), 5.13 (s, 2H), 5.04 (s, 2H), 4.44 (br s, 2H), 3.70 (br s, 2H), 3.39 (br s, 2H), 2.59 (s, 3H), 1.47 (s, 9H), 1.38 (s, 12H).

### tert-Butyl ((3-(3′-((2-chloro-5-((5-cyanopyridin-3-yl)methoxy)-4-formylphenoxy)methyl)-2,2′-dimethyl-[1,1′-biphenyl]-3-yl)-4-oxo-4H-pyrido[1,2-a]pyrimidin-8-yl)methyl)(2-hydroxyethyl)carbamate

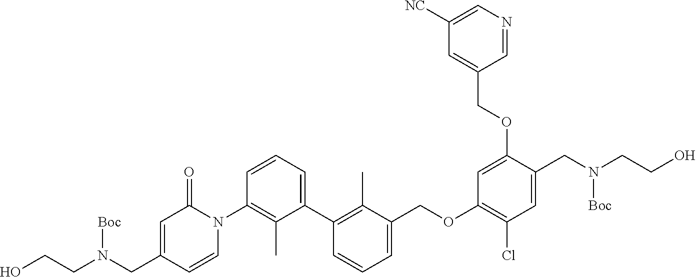 [SMILES: Cc1c(COc2cc(OCc3cncc(C#N)c3)c(CN(CCO)C(=O)OC(C)(C)C)cc2Cl)cccc1-c1cccc(-n2ccc(CN(CCO)C(=O)OC(C)(C)C)cc2=O)c1C]

To a solution of tert-butyl 5-chloro-2-((5-cyanopyridin-3-yl)methoxy)-4-((2-methyl-3-(4,4,5,5-tetramethyl-1,3,2-dioxaborolan-2-yl)benzyl)oxy)benzyl(2-hydroxyethyl)carbamate (400 mg, 0.6 mmol) and tert-butyl ((1-(3-bromo-2-methylphenyl)-2-oxo-1,2-dihydropyridin-4-yl)methyl)(2-hydroxyethyl)carbamate (280 mg, 0.64 mmol) in H₂O (5 mL) and THE (20 mL) was added [2-(2-aminophenyl)phenyl]-chloro-palladium; bis(1-adamantyl)-butyl-phosphane (70 mg, 0.1 mmol), cesium fluoride (93 mg, 0.6 mmol) and potassium phosphate (320 mg, 1.5 mmol). The mixture was stirred at 80° C. under nitrogen gas for 12 hrs. The mixture was combined with three batches at 20 mg scale and one batch at 50 mg scale. The mixture was diluted with THE (30 mL), washed with saturated aqueous brine solution (2×10 mL), dried over anhydrous sodium sulfate, filtered and concentrated under reduced pressure. The residue was purified by normal phase flash SiO₂ chromatography (90-0% petroleum ether/50% THE in ethyl acetate) to give the semi-purified product (400 mg) as a yellow solid. The solid was further purified by reverse phase HPLC to afford tert-butyl N-[[1-[3-[3-[[4-[[tert-butoxycarbonyl(2-hydroxyethyl)amino]methyl]-2-chloro-5-[(5-cyano-3-pyridyl)methoxy]phenoxy]methyl]-2-methyl-phenyl]-2-methyl-phenyl]-2-oxo-4-pyridyl]methyl]-N-(2-hydroxyethyl)carbamate (300 mg, 44% yield in total, m/z: 894 [M+H]⁺ observed) as a yellow solid. ¹H NMR (400 MHz, CDCl₃): δ 9.02 (d, J=1.6 Hz, 1H), 8.97 (s, 1H), 8.44 (br s, 1H), 7.57 (t, J=6.4 Hz, 1H), 7.51 (br s, 1H), 7.45-7.38 (m, 1H), 7.35-7.20 (m, 3H), 7.17-7.09 (m, 3H), 6.28-6.14 (m, 2H), 5.33 (s, 2H), 5.29 (br s, 2H), 4.72 (t, J=5.2 Hz, 1H), 4.65 (br s, 1H), 4.40-4.31 (m, 4H), 3.54-3.50 (m, 2H), 3.44-3.39 (m, 2H), 2.08 (d, J=5.6 Hz, 3H), 1.69 (d, J=3.2 Hz, 3H), 1.48-1.36 (m, 9H), 1.34-1.24 (m, 9H).

### 5-((4-Chloro-2-(((2-hydroxyethyl)amino)methyl)-5-((3′-(4-(((2-hydroxyethyl)amino)methyl)-2-oxopyridin-1(2H)-yl)-2,2′-dimethyl-[1,1′-biphenyl]-3-yl)methoxy)phenoxy)methyl)nicotinonitrile

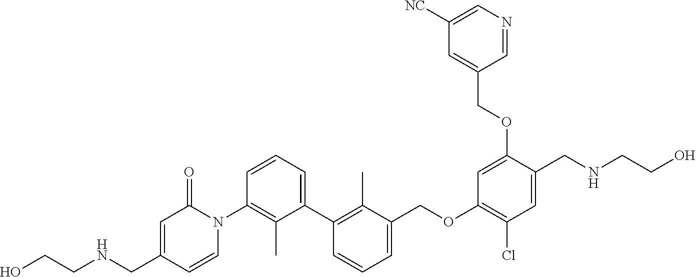 [SMILES: Cc1c(COc2cc(OCc3cncc(C#N)c3)c(CNCCO)cc2Cl)cccc1-c1cccc(-n2ccc(CNCCO)cc2=O)c1C]

To a solution of tert-butyl N-[[1-[3-[3-[[4-[[tert-butoxycarbonyl(2-hydroxyethyl)amino]methyl]-2-chloro-5-[(5-cyano-3-pyridyl)methoxy]phenoxy]methyl]-2-methyl-phenyl]-2-methyl-phenyl]-2-oxo-4-pyridyl]methyl]-N-(2-hydroxyethyl)carbamate (280 mg, 0.31 mmol) in THE (5 mL) was added HCl (4 M solution in ethyl acetate, 3 mL, 12 mmol) at 0° C. The mixture was stirred at r.t. under nitrogen gas for 2 hrs. The mixture was concentrated. To the residue was added THE (20 mL) and the mixture was neutralized with saturated aqueous sodium bicarbonate solution. The mixture was separated and the organic phase was washed with saturated aqueous brine solution (2×5 mL), dried over anhydrous sodium sulfate, filtered and concentrated under reduced pressure. The residue was purified by reverse phase HPLC to afford 5-((4-chloro-2-(((2-hydroxyethyl)amino)methyl)-5-((3′-(4-(((2-hydroxyethyl)amino)methyl)-2-oxopyridin-1(2H)-yl)-2,2′-dimethyl-[1,1′-biphenyl]-3-yl)methoxy)phenoxy)methyl)nicotinonitrile (58 mg, 25% yield, m/z: 694 [M+H]⁺, 348 [M/2+H]⁺ observed) as a brown solid. ¹H NMR (400 MHz, CD₃OD): δ8.94 (s, 1H), 8.90 (d, J=1.2 Hz, 1H), 8.34 (br d, J=1.6 Hz, 1H), 7.56-7.47 (m, 2H), 7.46-7.40 (m, 1H), 7.34 (d, J=1.6 Hz, 1H), 7.30-7.23 (m, 3H), 7.22-7.09 (m, 1H), 6.98 (s, 1H), 6.66 (s, 1H), 6.56 (t, J=6.8 Hz, 1H), 5.29 (s, 2H), 5.26 (br d, J=2.8 Hz, 2H), 3.77 (s, 4H), 3.71-3.68 (m, 2H), 3.64 (t, J=5.6 Hz, 2H), 2.77 (t, J=5.6 Hz, 2H), 2.70 (br t, J=5.2 Hz, 2H), 2.15 (d, J=16.0 Hz, 3H), 1.77 (d, J=1.2 Hz, 3H).

### Example 24: 5-((4-Chloro-5-((2,2′-dimethyl-3′-(4-oxo-3-(((((S)-5-oxopyrrolidin-2-yl)methyl)amino)methyl)-4H-quinolizin-8-yl)-[1,1′-biphenyl]-3-yl)methoxy)-2-(((((S)-5-oxopyrrolidin-2-yl)methyl)amino)methyl)phenoxy)methyl)nicotinonitrile

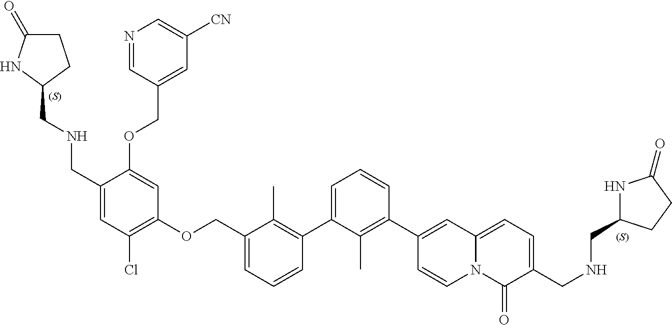

To a solution of 5-((4-chloro-2-formyl-5-((3′-(3-formyl-4-oxo-4H-quinolizin-8-yl)-2,2′-dimethyl-[1,1′-biphenyl]-3-yl)methoxy)phenoxy)methyl)nicotinonitrile, (400 mg, 0.61 mmol) and (S)-5-(aminomethyl)pyrrolidin-2-one as hydrochloride salt (698 mg, 6.12 mmol) in THE (10 mL) and MeOH (10 mL) was added glacial AcOH (0.03 mL, 0.61 mmol). The reaction mixture was stirred at r.t. for 50 min. Sodium cyanoborohydride (192 mg, 3.06 mmol) was added to the mixture. The mixture was stirred at r.t. for 10 min. The reaction mixture was filtered and concentrated. The residue was purified by reverse phase HPLC to give 5-((4-chloro-5-((2,2′-dimethyl-3′-(4-oxo-3-(((((S)-5-oxopyrrolidin-2-yl)methyl)amino)methyl)-4H-quinolizin-8-yl)-[1,1′-biphenyl]-3-yl)methoxy)-2-(((((S)-5-oxopyrrolidin-2-yl)methyl)amino)methyl)phenoxy)methyl)nicotinonitrile as a formic acid salt (102 mg, 17% yield, m/z: 850 [M+H]⁺ observed (MS: Method A)) as a yellow solid. 12.29 mg of 5-((4-chloro-5-((2,2′-dimethyl-3′-(4-oxo-3-(((((S)-5-oxopyrrolidin-2-yl)methyl)amino)methyl)-4H-quinolizin-8-yl)-[1,1′-biphenyl]-3-yl)methoxy)-2-(((((S)-5-oxopyrrolidin-2-yl)methyl)amino)methyl)phenoxy)methyl)nicotinonitrile was delivered. ¹H NMR (400 MHz, CD₃OD) δ9.18 (d, J=7.6 Hz, 1H), 8.93 (s, 1H), 8.91 (s, 1H), 8.32 (s, 1H), 7.93 (d, J=7.6 Hz, 1H), 7.79 (s, 1H), 7.47 (d, J=8 Hz, 1H), 7.43-7.37 (m, 4H), 7.29-7.22 (m, 2H), 7.15 (d, J=8 Hz, 1H), 7.05-7.00 (m, 2H), 5.33 (s, 2H), 5.29 (s, 2H), 4.29-4.21 (m, 2H), 4.02-3.84 (m, 4H), 3.11-3.03 (m, 2H), 2.84-2.82 (m, 2H), 2.41-2.23 (m, 6H), 2.15 (s, 3H), 1.99 (s, 3H), 1.89-1.74 (m, 2H).

### Example 25: 5-((4-Chloro-5-((2′-cyano-3′-(3-(((2-hydroxyethyl)amino)methyl)-4-oxo-4H-pyrido[1,2-a]pyrimidin-8-yl)-2-methyl-[1,1′-biphenyl]-3-yl)methoxy)-2-(((2-hydroxyethyl)amino)methyl)phenoxy)methyl)nicotinonitrile

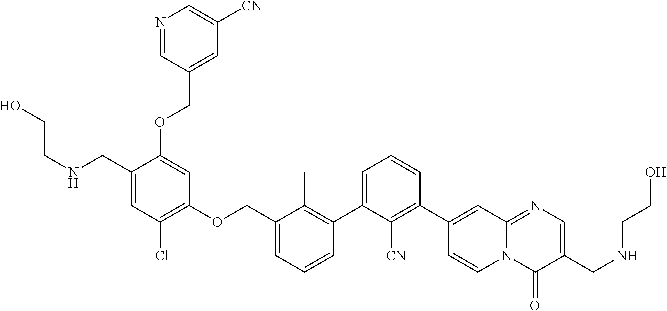 [SMILES: Cc1c(COc2cc(OCc3cncc(C#N)c3)c(CNCCO)cc2Cl)cccc1-c1cccc(-c2ccn3c(=O)c(CNCCO)cnc3c2)c1C#N]

### 2-Bromo-6-(3-(hydroxymethyl)-4-oxo-4H-pyrido[1,2-a]pyrimidin-8-yl)benzonitrile

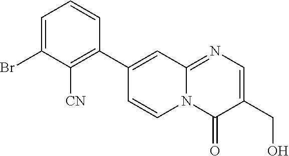 [SMILES: N#Cc1c(Br)cccc1-c1ccn2c(=O)c(CO)cnc2c1]

To a mixture of 8-bromo-3-(hydroxymethyl)-4H-pyrido[1,2-a]pyrimidin-4-one (1 g, 4 mmol) and (3-bromo-2-cyanophenyl)boronic acid (0.9 g, 4 mmol) in dioxane/H₂O mixture (5:1 (v v), 19 mL) was added potassium carbonate (1.6 g, 12 mmol) and 1,1′-bis(diphenylphosphino)ferrocene-palladium(II)dichloride dichloromethane complex (0.3 g, 0.4 mmol). The mixture was stirred at 80° C. for 1 hour. The mixture was combined with another eight batches at the same scale. The combined mixture was treated with H₂O (80 mL) and extracted with EtOAc/THF mixture (1:1 (v v), 40 mL). The combined organic layers were concentrated under reduced pressure to give a residue. The residue was purified by reverse phase HPLC to afford 2-bromo-6-(3-(hydroxymethyl)-4-oxo-4H-pyrido[1,2-a]pyrimidin-8-yl)benzonitrile as a yellow solid (1.3 g, 12% yield). ¹H NMR (400 MHz, CDCl₃): δ 9.07 (d, J=7.2 Hz, 1H), 8.33 (s, 1H), 7.78 (d, J=6.8 Hz, 1H), 7.72 (d, J=1.6 Hz, 1H), 7.52 (t, J=8 Hz, 1H), 7.48 (d, J=6.8 Hz, 1H), 7.28 (dd, J=2 Hz, J=5.6 Hz, 1H), 4.71 (s, 2H), 3.42 (s, 1H).

### 2-Bromo-6-(3-(chloromethyl)-4-oxo-4H-pyrido[1,2-a]pyrimidin-8-yl)benzonitrile

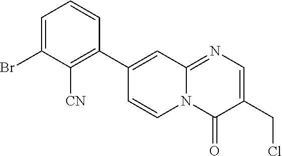 [SMILES: N#Cc1c(Br)cccc1-c1ccn2c(=O)c(CCl)cnc2c1]

To a mixture of 2-bromo-6-(3-(hydroxymethyl)-4-oxo-4H-pyrido[1,2-a]pyrimidin-8-yl) benzonitrile (0.8 g, 2.25 mmol) in DCM (16 mL) was added sulfurous dichloride (2.7 g, 22.5 mmol). The mixture was stirred at 50° C. for 5 hours. The reaction mixture was concentrated under reduced pressure to give 2-bromo-6-(3-(chloromethyl)-4-oxo-4H-pyrido[1,2-a]pyrimidin-8-yl)benzonitrile as a black solid (0.8 g, crude) which was used to next step directly.

### 2-Bromo-6-(3-(((2-hydroxyethyl)amino)methyl)-4-oxo-4H-pyrido[1,2-a]pyrimidin-8-yl)benzonitrile

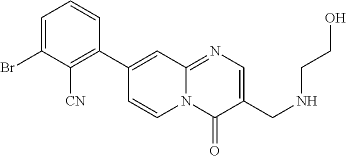 [SMILES: N#Cc1c(Br)cccc1-c1ccn2c(=O)c(CNCCO)cnc2c1]

To a mixture of 2-bromo-6-(3-(chloromethyl)-4-oxo-4H-pyrido[1,2-a]pyrimidin-8-yl)benzonitrile (0.8 g, 2.1 mmol) and 2-aminoethanol (0.65 g, 10.7 mmol) in THF/DMF mixture (1:1, 16 mL) was added potassium carbonate (1.5 g, 10.7 mmol). The mixture was stirred at 60° C. for 3 hours. The crude product 2-bromo-6-(3-(((2-hydroxyethyl)amino)methyl)-4-oxo-4H-pyrido[1,2-a]pyrimidin-8-yl)benzonitrile as a black mixture (16 mL) was used to next step directly. (m/z: 399 [M]⁺, 401 [M+2]⁺ observed).

### tert-Butyl ((8-(3-bromo-2-cyanophenyl)-4-oxo-4H-pyrido[1,2-a]pyrimidin-3-yl)methyl)(2-hydroxyethyl)carbamate

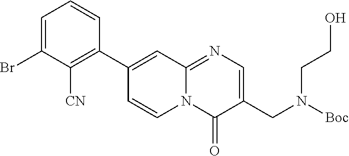 [SMILES: CC(C)(C)OC(=O)N(CCO)Cc1cnc2cc(-c3cccc(Br)c3C#N)ccn2c1=O]

A mixture of 2-bromo-6-(3-(((2-hydroxyethyl)amino)methyl)-4-oxo-4H-pyrido[1,2-a]pyrimidin-8-yl)benzonitrile in THF/DMF mixture (1:1, 16 mL, 2 mmol) and di-tert-butyl dicarbonate (3 g, 14 mmol) was stirred at 25° C. for 20 min. The reaction mixture was treated with H₂O (30 mL) and extracted with EtOAc (3×50 mL). The organic layers were concentrated under reduced pressure to give a residue. The residue was purified by normal phase SiO₂ chromatography (10-50% EtOAc/petroleum ether) to afford tert-butyl ((8-(3-bromo-2-cyanophenyl)-4-oxo-4H-pyrido[1,2-a]pyrimidin-3-yl)methyl)(2-hydroxyethyl)carbamate (0.4 g, 40% yield) as a white solid. ¹H NMR (400 MHz, CDCl₃): δ 9.08 (d, J=7.6 Hz, 1H), 7.77 (d, J=8 Hz, 1H), 7.12 (d, J=1.2 Hz, 1H), 7.53 (t, J=8 Hz, 1H), 7.47 (d, J=8 Hz, 1H), 7.26 (dd, J=7.6 Hz, J=2 Hz, 1H), 4.44 (s, 2H), 3.74 (d, J=4.5 Hz, 2H), 3.57 (s, 2H), 1.41 (s, 1OH) (overlapped with solvent peak).

### tert-Butyl N-[[8-[3-[3-[[4-[[tert-butoxycarbonyl(2-hydroxyethyl)amino]methyl]-2-chloro-5-[(5-cyano-3-pyridyl)methoxy]phenoxy]methyl]-2-methyl-phenyl]-2-cyano-phenyl]-4-oxo-pyrido[1,2-a]pyrimidin-3-yl]methyl]-N-(2-hydroxyethyl)carbamate

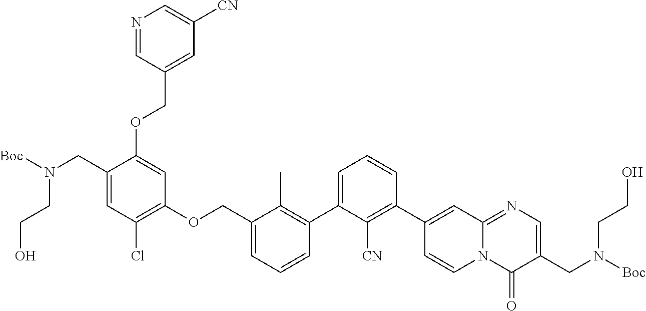 [SMILES: Cc1c(COc2cc(OCc3cncc(C#N)c3)c(CN(CCO)C(=O)OC(C)(C)C)cc2Cl)cccc1-c1cccc(-c2ccn3c(=O)c(CN(CCO)C(=O)OC(C)(C)C)cnc3c2)c1C#N]

A mixture of tert-butyl ((8-(3-bromo-2-cyanophenyl)-4-oxo-4H-pyrido[1,2-a]pyrimidin-3-yl)methyl)(2-hydroxyethyl)carbamate (0.1 g, 0.2 mmol) and tert-butyl ((8-(2-cyano-3-(4,4,5,5-tetramethyl-1,3,2-dioxaborolan-2-yl)phenyl)-4-oxo-4H-pyrido[1,2-a]pyrimidin-3-yl)methyl)(2-hydroxyethyl)carbamate (0.16 g, 0.24 mmol) in THF/H₂O mixture (3:1, 4 mL) was added potassium phosphate (0.08 g, 0.4 mmol) and [2-(2-aminophenyl)phenyl]-chloro-palladium;bis(1-adamantyl)-butyl-phosphane (0.01 g, 0.02 mmol). The mixture was stirred at 80° C. for 1 hour. The mixture was combined another batch at the same scale. To the combined mixture was added H₂O (20 mL). Then the mixture was extracted with EtOAc (2×20 mL). The combined organic layers were concentrated under reduced pressure to give a residue. The residue was purified by normal phase SiO₂ chromatography (20-100% EtOAc/petroleum ether) to afford tert-butyl N-[[8-[3-[3-[[4-[[tert-butoxycarbonyl(2-hydroxyethyl)amino]methyl]-2-chloro-5-[(5-cyano-3-pyridyl)methoxy]phenoxy]methyl]-2-methyl-phenyl]-2-cyano-phenyl]-4-oxo-pyrido[1,2-a]pyrimidin-3-yl]methyl]-N-(2-hydroxyethyl)carbamate as a white solid (0.18 g, 49% yield). ¹H NMR (400 MHz, CDCl₃): δ 9.10 (d, J=7.2 Hz, 1H), 8.73 (dd, J=1.6 Hz, J=11.6 Hz, 1H), 8.49 (brs, 1H), 8.34 (brs, 1H), 7.92 (brs, 1H), 7.81 (s, 1H), 7.75 (t, J=7.6 Hz, 1H), 7.56 (d, J=7.6 Hz, 1H), 7.49 (d, J=8 Hz, 1H), 7.39 (d, J=7.2 Hz, 1H), 7.34 (d, J=7.2 Hz, 1H), 7.25 (t, J=7.6 Hz, 1H), 7.23-7.20 (m, 1H), 6.35 (s, 1H), 5.16 (dd, J=12.8 Hz, J=16.4 Hz, 2H), 4.89 (q, J=12.8 Hz, 2H), 4.44-4.38 (m, 4H), 3.75 (s, 2H), 3.62-3.58 (m, 4H), 3.30 (s, 2H), 1.39 (s, 18H).

### 5-((4-Chloro-5-((2′-cyano-3′-(3-(((2-hydroxyethyl)amino)methyl)-4-oxo-4H-pyrido[1,2-a]pyrimidin-8-yl)-2-methyl-[1,1′-biphenyl]-3-yl)methoxy)-2-(((2-hydroxyethyl)amino)methyl)phenoxy)methyl)nicotinonitrile

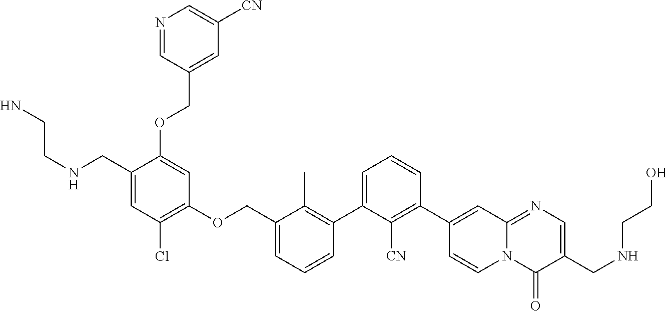 [SMILES: Cc1c(COc2cc(OCc3cncc(C#N)c3)c(CNCCN)cc2Cl)cccc1-c1cccc(-c2ccn3c(=O)c(CNCCO)cnc3c2)c1C#N]

A mixture of tert-butyl N-[[8-[3-[3-[[4-[[tert-butoxycarbonyl(2-hydroxyethyl)amino]methyl]-2-chloro-5-[(5-cyano-3-pyridyl)methoxy]phenoxy]methyl]-2-methyl-phenyl]-2-cyano-phenyl]-4-oxo-pyrido[1,2-a]pyrimidin-3-yl]methyl]-N-(2-hydroxyethyl)carbamate (0.15 g, 0.16 mmol) in DCM (5 mL) was added trifluoroacetic acid (1 mL, 13.5 mmol). The mixture was stirred at r.t. for 0.5 hour. The reaction mixture was treated with H₂O (10 mL) and extracted with EtOAc (3×10 mL). The combined organic layers were concentrated under reduced pressure to give a residue. The residue was purified by reverse phase HPLC to afford 5-((4-chloro-5-((2′-cyano-3′-(3-(((2-hydroxyethyl)amino)methyl)-4-oxo-4H-pyrido[1,2-a]pyrimidin-8-yl)-2-methyl-[1,1′-bipenyl]-3-yl)methoxy)-2-(((2-hydroxyethyl)amino)methyl)phenoxy)methyl)nicotinonitrile as a white solid. (31.67 mg, 27% yield, m/z: 756 [M+H]⁺ observed). ¹H NMR (400 MHz, CD₃OD): δ9.21 (d, J=7.6 Hz, 1H), 8.89 (dd, J=2 Hz, J=6.8 Hz, 2H), 8.50 (s, 2H), 8.30 (s, 1H), 7.99 (d, J=1.2 Hz, 1H), 7.94 (t, J=8 Hz, 1H), 7.82 (d, J=8 Hz, 1H), 7.69 (dd, J=2 Hz, J=7.6 Hz, 1H), 7.63 (d, J=7.6 Hz, 1H), 7.53 (d, J=6.8 Hz, 1H), 7.49 (s, 1H), 7.31-7.30 (m, 1H), 6.92 (s, 1H), 5.38 (s, 2H), 5.27 (d, J=7.6 Hz, 2H), 4.22 (s, 2H), 4.14 (s, 2H), 3.81 (t, J=5.2 Hz, 2H), 3.72 (t, J=5.2 Hz, 2H), 3.12 (t, J=5.2 Hz, 2H), 3.03 (t, J=5.2 Hz, 2H), 2.28 (s, 3H).

### Example 26: 5-((4-Chloro-5-((3′-(1-(2-(3-(hydroxymethyl)azetidin-1-yl)ethyl)-4-methyl-2,5-dioxo-2,3,4,5-tetrahydro-1H-benzo[e][1,4]diazepin-7-yl)-2,2′-dimethyl-[1,1′-biphenyl]-3-yl)methoxy)-2-((3-(hydroxymethyl)azetidin-1-yl)methyl)phenoxy)methyl)nicotinonitrile

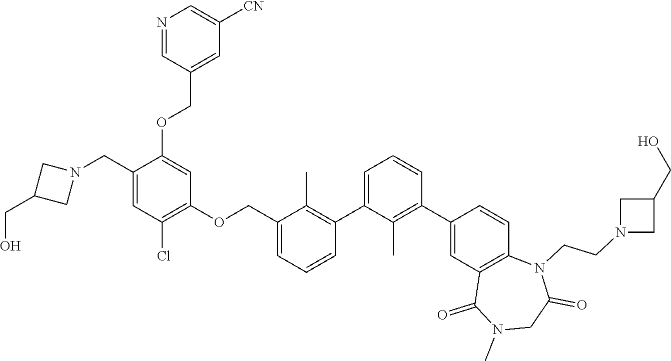 [SMILES: Cc1c(COc2cc(OCc3cncc(C#N)c3)c(CN3CC(CO)C3)cc2Cl)cccc1-c1cccc(-c2ccc3c(c2)C(=O)N(C)CC(=O)N3CCN2CC(CO)C2)c1C]

To a mixture of 5-((4-chloro-5-((2,2′-dimethyl-3′-(4-methyl-2,5-dioxo-1-(2-oxoethyl)-2,3,4,5-tetrahydro-1H-benzo[e][1,4]diazepin-7-yl)-[1,1′-biphenyl]-3-yl)methoxy)-2-formylphenoxy)methyl)nicotinonitrile (0.3 g, 0.42 mmol) and 4 Å molecular sieve (0.5 g) in DCM (30 mL) was added azetidin-3-ylmethanol as hydrochloride salt (1.56 g, 12.6 mmol) and TEA (2.2 mL, 15.8 mmol). The mixture was stirred at 25° C. for 11 h, and then sodium triacetoxyborohydride (1.8 g, 8.4 mmol) was added and the mixture was stirred for 1 min. The reaction mixture was filtered and the filtrate was concentrated under reduced pressure to give a residue. The residue was purified by reverse phase HPLC to afford 5-((4-chloro-5-((3′-(1-(2-(3-(hydroxymethyl)azetidin-1-yl)ethyl)-4-methyl-2,5-dioxo-2,3,4,5-tetrahydro-1H-benzo[e][1,4]diazepin-7-yl)-2,2′-dimethyl-[1,1′-biphenyl]-3-yl)methoxy)-2-((3-(hydroxymethyl)azetidin-1-yl)methyl)phenoxy)methyl)nicotinonitrile as a formic acid salt (62.28 mg, 17% yield, m/z: 855 [M+H]⁺ observed). ¹H NMR (400 MHz, CD₃OD): δ8.96 (m, 2H), 8.55 (br, 1H), 8.40 (s, 1H), 7.78 (s, 1H), 7.65 (d, J=8.4 Hz, 1H), 7.57 (d, J=8.4 Hz, 1H), 7.48 (m, 2H), 7.35 (t, J=7.6 Hz, 1H), 7.29 (m, 2H), 7.17 (m, 2H), 7.07 (s, 1H), 5.38 (s, 2H), 5.32 (s, 2H), 4.23 (m, 4H), 4.01 (m, 2H), 3.89 (m, 3H), 3.79 (m, 1H), 3.65 (m, 6H), 3.40 (br, 2H), 3.28 (s, 3H), 2.97 (br, 3H), 2.74 (m, 1H), 2.16 (s, 3H), 1.94 (s, 3H).

### Example 27: 5-((4-Chloro-5-(((2,2′-dimethyl-3′-(4-methyl-2,5-dioxo-1-(2-((((S)-5-oxopyrrolidin-2-yl)methyl)amino)ethyl)-2,3,4,5-tetrahydro-1H-benzo[e][1,4]diazepin-7-yl)-[1,1′-biphenyl]-3-yl)oxy)methyl)-2-(((((S)-5-oxopyrrolidin-2-yl)methyl)amino)methyl)phenoxy)methyl)nicotinonitrile

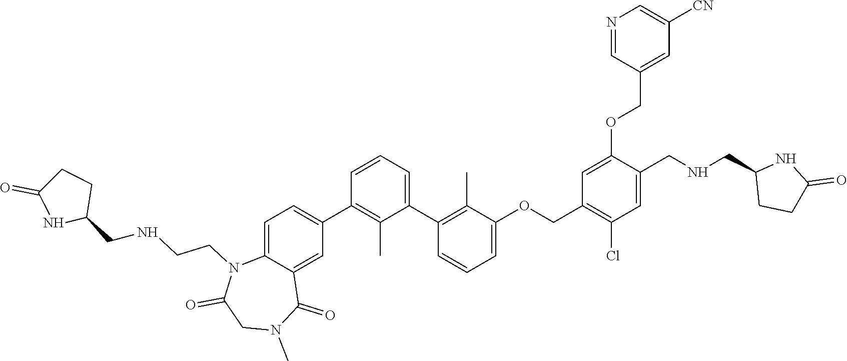 [SMILES: Cc1c(OCc2cc(OCc3cncc(C#N)c3)c(CNC[C@@H]3CCC(=O)N3)cc2Cl)cccc1-c1cccc(-c2ccc3c(c2)C(=O)N(C)CC(=O)N3CCNC[C@@H]2CCC(=O)N2)c1C]

### 1-(2,2-Dimethoxyethyl)-4-methyl-7-(2-methyl-3-(4,4,5,5-tetramethyl-1,3,2-dioxaborolan-2-yl)phenyl)-3,4-dihydro-1H-benzo[e][1,4]diazepine-2,5-dione

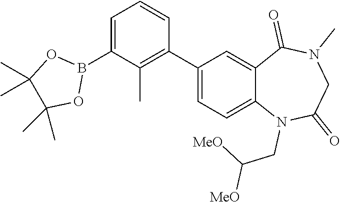 [SMILES: COC(CN1C(=O)CN(C)C(=O)c2cc(-c3cccc(B4OC(C)(C)C(C)(C)O4)c3C)ccc21)OC]

To a solution of 7-(3-bromo-2-methylphenyl)-1-(2,2-dimethoxyethyl)-4-methyl-3,4-dihydro-1H-benzo[e][1,4]diazepine-2,5-dione (1 g, 2.24 mmol) and 4,4,4′,4′,5,5,5′,5′-octamethyl-2,2′-bi(1,3,2-dioxaborolane) (1.14 g, 4.47 mmol) in dioxane (10 mL) was added potassium acetate (0.43 g, 4.47 mmol) and [1,1-bis(diphenylphosphino)ferrocene]palladium(II)chloride (0.18 g, 0.22 mmol). The mixture was stirred at 80° C. for 30 min. To the mixture was added water (5 mL). The mixture was extracted with ethyl acetate (2×5 mL). The combined organic phase was dried with anhydrous sodium sulfate, filtered and concentrated to give the residue. The residue was purified by normal phase SiO₂ chromatography (0-80% EtOAc/petroleum ether) to afford 1-(2,2-dimethoxyethyl)-4-methyl-7-(2-methyl-3-(4,4,5,5-tetramethyl-1,3,2-dioxaborolan-2-yl)phenyl)-3,4-dihydro-1H-benzo[e][1,4]diazepine-2,5-dione (0.6 g, 54% yield, m/z: 463 [M-OMe]⁺ observed) as a yellow solid.

### 5-((4-Chloro-5-(((3′-(1-(2,2-dimethoxyethyl)-4-methyl-2,5-dioxo-2,3,4,5-tetrahydro-1H-benzo[e][1,4]diazepin-7-yl)-2,2′-dimethyl-[1,1′-biphenyl]-3-yl)oxy)methyl)-2-formylphenoxy)methyl)nicotinonitrile

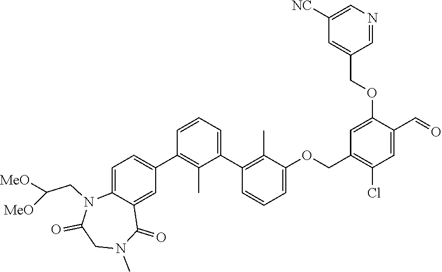 [SMILES: COC(CN1C(=O)CN(C)C(=O)c2cc(-c3cccc(-c4cccc(OCc5cc(OCc6cncc(C#N)c6)c(C=O)cc5Cl)c4C)c3C)ccc21)OC]

To a solution of 5-((5-((3-bromo-2-methylphenoxy)methyl)-4-chloro-2-formylphenoxy)methyl)nicotinonitrile (300 mg, 635 umol) and 1-(2,2-dimethoxyethyl)-4-methyl-7-(2-methyl-3-(4,4,5,5-tetramethyl-1,3,2-dioxaborolan-2-yl)phenyl)-3,4-dihydro-1H-benzo[e][1,4]diazepine-2,5-dione (471 mg, 953 umol) in THE (2 mL) and H₂O (1 mL) was added [2-(2-aminophenyl)phenyl]-chloro-palladium;bis(1-adamantyl)-butyl-phosphane (42.5 mg, 63.6 umol) and potassium phosphate (270 mg, 127 umol). The mixture was stirred at 80° C. for 1 hr. To the mixture was added water (5 mL). The mixture was extracted with ethyl acetate (2×5 mL). The combined organic phase was dried with anhydrous sodium sulfate, filtered and concentrated to give the residue. The residue was purified by normal phase SiO₂ chromatography (0-50% EtOAc/petroleum ether) to afford 5-((4-chloro-5-(((3′-(1-(2,2-dimethoxyethyl)-4-methyl-2,5-dioxo-2,3,4,5-tetrahydro-1H-benzo[e][1,4]diazepin-7-yl)-2,2′-dimethyl-[1,1′-biphenyl]-3-yl)oxy)methyl)-2-formylphenoxy)methyl)nicotinonitrile (180 mg, 19% yield, m/z: 759 [M+H]⁺ observed) as a yellow solid.

### 5-((4-Chloro-5-(((2,2′-dimethyl-3′-(4-methyl-2,5-dioxo-1-(2-oxoethyl)-2,3,4,5-tetrahydro-1H-benzo[e][1,4]diazepin-7-yl)-[1,1′-biphenyl]-3-yl)oxy)methyl)-2-formylphenoxy)methyl)nicotinonitrile

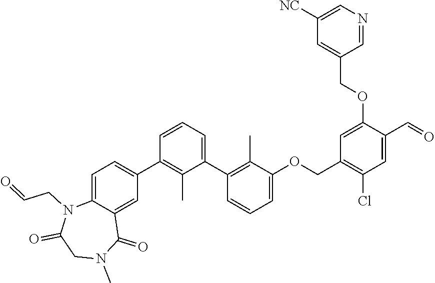 [SMILES: Cc1c(OCc2cc(OCc3cncc(C#N)c3)c(C=O)cc2Cl)cccc1-c1cccc(-c2ccc3c(c2)C(=O)N(C)CC(=O)N3CC=O)c1C]

To a solution of 5-((4-chloro-5-(((3′-(1-(2,2-dimethoxyethyl)-4-methyl-2,5-dioxo-2,3,4,5-tetrahydro-1H-benzo[e][1,4]diazepin-7-yl)-2,2′-dimethyl-[1,1′-biphenyl]-3-yl)oxy)methyl)-2-formylphenoxy)methyl)nicotinonitrile (40 mg, 52.6 umol) in 6 M HCl solution in EtOAc (5 mL). The mixture was stirred at r.t. for 5 min. The reaction mixture combined with another batch at same scale was concentrated under reduced pressure to afford 5-((4-chloro-5-(((2,2′-dimethyl-3′-(4-methyl-2,5-dioxo-1-(2-oxoethyl)-2,3,4,5-tetrahydro-1H-benzo[e][1,4]diazepin-7-yl)-[1,1′-biphenyl]-3-yl)oxy)methyl)-2-formylphenoxy)methyl)nicotinonitrile (80 mg, crude, m/z: 760 [M+H]⁺ observed) as a yellow solid.

### 5-((4-Chloro-5-(((2,2′-dimethyl-3′-(4-methyl-2,5-dioxo-1-(2-((((S)-5-oxopyrrolidin-2-yl)methyl)amino)ethyl)-2,3,4,5-tetrahydro-1H-benzo[e][1,4]diazepin-7-yl)-[1,1′-biphenyl]-3-yl)oxy)methyl)-2-(((((S)-5-oxopyrrolidin-2-yl)methyl)amino)methyl)phenoxy)methyl)nicotinonitrile

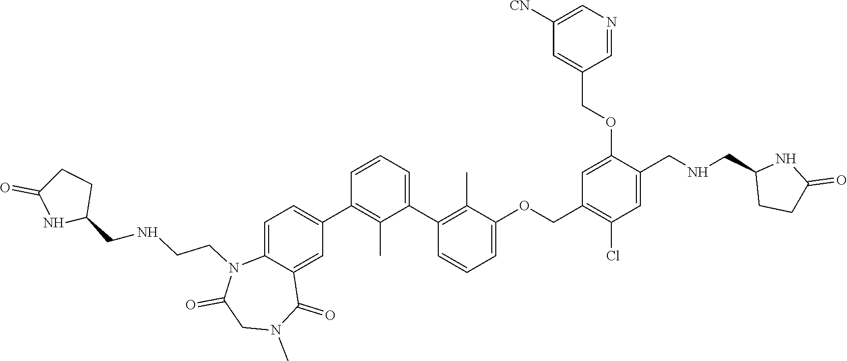

To a solution of 5-((4-chloro-5-(((2,2′-dimethyl-3′-(4-methyl-2,5-dioxo-1-(2-oxoethyl)-2,3,4,5-tetrahydro-1H-benzo[e][1,4]diazepin-7-yl)-[1,1′-biphenyl]-3-yl)oxy)methyl)-2-formylphenoxy)methyl)nicotinonitrile (40 mg, 56.1 umol) in DCM (3 mL) was added triethylamine (781 uL, 561 umol) and molecular sieves pack 4 Å powder (500 mg) and (S)-5-(aminomethyl)pyrrolidin-2-one hydrochloride (128 mg, 850 umol). The mixture was stirred at r.t. for 12 hr. Sodium cyanoborohydride (35 mg, 561 umol) was added to the mixture. The mixture was stirred at r.t. for 1 hr. The reaction mixture combined with another batch at same scale was filtered and the filtrate was concentrated under reduced pressure to give a residue. The residue was purified by reverse phase HPLC to afford 5-((4-chloro-5-(((2,2′-dimethyl-3′-(4-methyl-2,5-dioxo-1-(2-((((S)-5-oxopyrrolidin-2-yl)methyl)amino)ethyl)-2,3,4,5-tetrahydro-1H-benzo[e][1,4]diazepin-7-yl)-[1,1′-biphenyl]-3-yl)oxy)methyl)-2-(((((S)-5-oxopyrrolidin-2-yl)methyl)amino)methyl)phenoxy)methyl)nicotinonitrile (6.37 mg, 6% yield, m/z: 909 [M+H]⁺ observed) as a white solid. ¹H NMR (400 MHz, CD₃OD): δ8.90 (d, J=6.8 Hz, 2H), 8.37 (m, 1H), 8.30 (s, 1H), 7.78 (s, 1H), 7.66 (m, 1H), 7.59 (d, J=8 Hz, 1H), 7.53 (s, 1H), 7.33 (t, J=7.2 Hz, 2H), 7.28 (d, J=8 Hz, 1H), 7.20 (t, J=7.6 Hz, 1H), 7.15 (d, J=7.6 Hz, 1H), 6.99 (d, J=8 Hz, 1H), 6.80 (d, J=7.6 Hz, 1H), 5.33 (s, 2H), 5.24 (s, 2H), 4.58 (m, 1H), 4.42 (m, 1H), 4.25 (d, J=14.8 Hz, 1H), 4.01 (s, 2H), 2H), 3.88 (m, 2H), 3.78 (d, J=14.8 Hz, 1H), 3.27 (m, 3H), 2.86 (m, 4H), 2.66 (m, 2H), 2.29 (m, 6H), 1.97 (m, 6H), 1.79 (m, 2H).

### Example 28: 5-((4-Chloro-5-((2,2′-dimethyl-3′-(4-oxo-3-(((((R)-5-oxopyrrolidin-2-yl)methyl)amino)methyl)-4H-quinolizin-8-yl)-[1,1′-biphenyl]-3-yl)methoxy)-2-(((((R)-5-oxopyrrolidin-2-yl)methyl)amino)methyl)phenoxy)methyl)nicotinonitrile

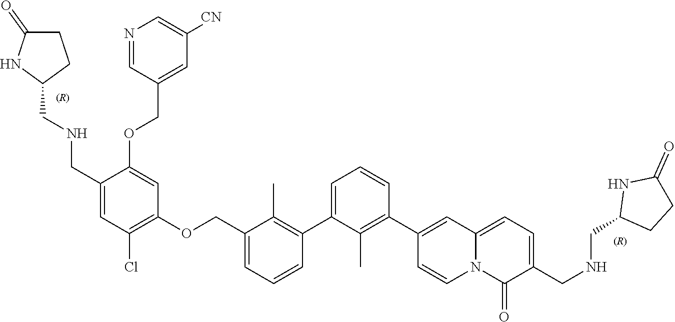

To a solution of 5-((4-chloro-2-formyl-5-((3′-(3-formyl-4-oxo-4H-quinolizin-8-yl)-2,2′-dimethyl-[1,1′-biphenyl]-3-yl)methoxy)phenoxy)methyl)nicotinonitrile (300 mg, 0.46 mmol) and (5R)-5-(aminomethyl)pyrrolidin-2-one as hydrochloride salt (552 mg, 3.67 mmol) in THE (10 mL) and MeOH (10 mL) was added 4 Å molecular sieve (200 mg) and glacial AcOH (0.03 mL, 0.46 mmol). The mixture was stirred at r.t. for 1.5 hr. Sodium cyanoborohydride (144.10 mg, 2.29 mmol) was added and the mixture was stirred at r.t. for 0.5 hr. The reaction mixture was filtered and concentrated. The residue was purified by reverse phase HPLC to give 5-((4-chloro-5-((2,2′-dimethyl-3′-(4-oxo-3-(((((R)-5-oxopyrrolidin-2-yl)methyl)amino)methyl)-4H-quinolizin-8-yl)-[1,1′-biphenyl]-3-yl)methoxy)-2-(((((R)-5-oxopyrrolidin-2-yl)methyl)amino)methyl)phenoxy)methyl)nicotinonitrile as a formic acid salt (51 mg, m/z: 850 [M+H]⁺ observed) as a yellow solid. ¹H NMR (400 MHz, CD₃OD) δ 9.19 (d, J=7.2 Hz, 1H), 8.94 (d, J=1.6 Hz, 1H), 8.91 (d, J=1.6 Hz, 1H), 8.43 (s, 1H), 8.33 (s, 1H), 7.93 (d, J=8.4 Hz, 1H), 7.80 (s, 1H), 7.48 (d, J=7.6 Hz, 1H), 7.41-7.37 (m, 4H), 7.28 (t, J=7.6 Hz, 1H), 7.23 (dd, J=7.6 Hz, J=2 Hz, 1H), 7.16 (d, J=7.2 Hz, 1H), 7.04 (d, J=8.4 Hz, 1H), 7.01 (s, 1H), 5.33 (s, 2H), 5.29 (s, 2H), 4.26-4.17 (m, 2H), 4.02-3.82 (m, 4H), 3.13-3.00 (m, 2H), 2.81-2.78 (m, 2H), 2.41-2.24 (m, 6H), 2.16 (s, 3H), 2.00 (s, 3H), 1.89-1.75 (m, 2H).

### Example 29: 1-((8-(3′-((4-((3-Carboxyazetidin-1-yl)methyl)-2-chloro-5-((5-cyanopyridin-3-yl)methoxy)phenoxy)methyl)-2,2′-dimethyl-[1,1′-biphenyl]-3-yl)-4-oxo-4H-quinolizin-3-yl)methyl)azetidine-3-carboxylic acid

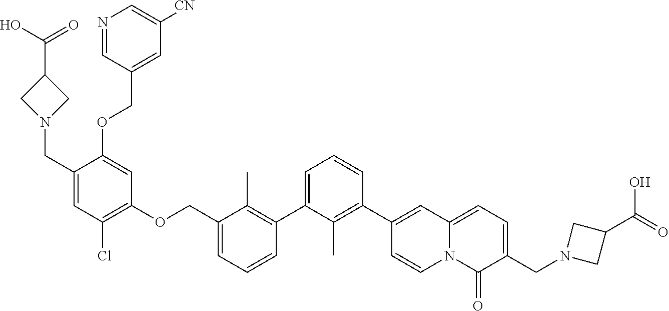

To a solution of 5-((4-chloro-2-formyl-5-((3′-(3-formyl-4-oxo-4H-quinolizin-8-yl)-2,2′-dimethyl-[1,1′-biphenyl]-3-yl)methoxy)phenoxy)methyl)nicotinonitrile (400 mg, 0.61 mmol) and azetidine-3-carboxylic acid (618 mg, 6.12 mmol) in THE (10 mL) and MeOH (10 mL) was added glacial AcOH (0.07 mL, 1.22 mmol). The mixture was stirred at r.t. for 1.5 hr. Sodium cyanoborohydride (153 mg, 2.45 mmol) was added to the mixture. The mixture was stirred at r.t. for 0.5 hr. The reaction mixture was filtered and concentrated. The residue was purified by reverse phase HPLC to give 1-((8-(3′-((4-((3-carboxyazetidin-1-yl)methyl)-2-chloro-5-((5-cyanopyridin-3-yl)methoxy)phenoxy)methyl)-2,2′-dimethyl-[1,1′-biphenyl]-3-yl)-4-oxo-4H-quinolizin-3-yl)methyl)azetidine-3-carboxylic acid as a formic acid salt (108.20 mg, m/z: 824 [M+H]⁺ observed) as a yellow solid. ¹H NMR (400 MHz, CD₃OD) δ9.17 (d, J=7.6 Hz, 1H), 8.95 (d, J=2 Hz, 1H), 8.92 (d, J=1.6 Hz, 1H), 8.36 (s, 1H), 7.96 (d, J=8 Hz, 1H), 7.80 (s, 1H), 7.51 (s, 1H), 7.47 (d, J=6.8 Hz, 1H), 7.41-7.37 (m, 3H), 7.28 (t, J=7.6 Hz, 1H), 7.23 (dd, J=6.8 Hz, J=2 Hz, 1H), 7.16 (d, J=6.8 Hz, 1H), 7.05 (s, 1H), 7.00 (d, J=8.4 Hz, 1H), 5.39 (s, 2H), 5.31 (s, 2H), 4.46 (s, 2H), 4.35 (s, 2H), 4.26 (d, J=9.6 Hz, 4H), 4.17 (d, J=8.4 Hz, 4H), 3.46 (m, 1H), 3.37 (m, 1H), 2.15 (s, 3H), 1.99 (s, 3H).

### Example 30: (S)-1-(4-((3′-(1-(2-((S)-3-Carboxypyrrolidin-1-yl)ethyl)-4-methyl-2,5-dioxo-2,3,4,5-tetrahydro-1H-benzo[e][1,4]diazepin-7-yl)-2,2′-dimethyl-[1,1′-biphenyl]-3-yl)methoxy)-5-chloro-2-((5-cyanopyridin-3-yl)methoxy)benzyl)pyrrolidine-3-carboxylic acid

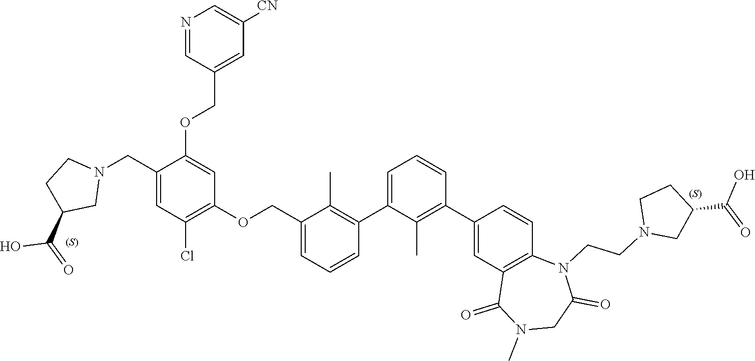

To a mixture of 5-((4-chloro-5-((2,2′-dimethyl-3′-(4-methyl-2,5-dioxo-1-(2-oxoethyl)-2,3,4,5-tetrahydro-1H-benzo[e][1,4]diazepin-7-yl)-[1,1′-biphenyl]-3-yl)methoxy)-2-formylphenoxy)methyl)nicotinonitrile (0.1 g, 0.14 mmol) and (S)-pyrrolidine-3-carboxylic acid as a hydrochloride salt (0.3 g, 2 mmol) in DCM (5 mL) was added triethylamine (0.03 mmol, 1.75 uL) and 4 Å molecular sieve (0.05 g). The mixture was stirred at r.t. for 12 hours under N₂. Sodium triacetoxyborohydride (0.6 g, 2.8 mmol) was added to the mixture and stirred at r.t. for 10 min. The mixture was combined with another four batches of the same scale. The combined mixture was filtered and concentrated to give the residue. The residue was purified by reverse phase HPLC to afford (S)-1-(4-((3′-(1-(2-((S)-3-carboxypyrrolidin-1-yl)ethyl)-4-methyl-2,5-dioxo-2,3,4,5-tetrahydro-1H-benzo[e][1,4]diazepin-7-yl)-2,2′-dimethyl-[1,1′-biphenyl]-3-yl)methoxy)-5-chloro-2-((5-cyanopyridin-3-yl)methoxy)benzyl)pyrrolidine-3-carboxylic acid as formic acid salt as a white solid (223.58 mg, 14% yield, m/z: 911 [M+H]⁺ observed). ¹H NMR (400 MHz, DMSO-d₆): δ 8.99 (dd, J=1.6 Hz, J=11.6 Hz, 2H), 8.43 (s, 1H), 8.17 (s, 1H), 7.68-7.62 (m, 3H), 7.51 (d, J=7.6 Hz, 1H), 7.37-7.29 (m, 4H), 7.15-7.13 (m, 3H), 5.33 (s, 2H), 5.28 (s, 2H), 4.15-4.12 (m, 2H), 4.14 (d, J=14.4 Hz, 2H), 3.84-3.56 (m, 9H), 2.92-2.82 (m, 4H), 2.70-2.64 (m, 2H), 2.34-2.32 (m, 2H), 2.11 (d, J=5.6 Hz, 3H), 1.96-1.85 (m, 7H).

### Example 31: 8-(3′-(6-Methoxy-5-(((((S)-5-oxopyrrolidin-2-yl)methyl)amino)methyl)pyridin-2-yl)-2,2′-dimethyl-[1,1′-biphenyl]-3-yl)-3-(((((S)-5-oxopyrrolidin-2-yl)methyl)amino)methyl)-4H-pyrido[1,2-a]pyrimidin-4-one

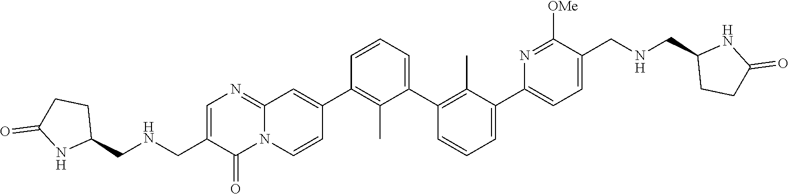 [SMILES: COc1nc(-c2cccc(-c3cccc(-c4ccn5c(=O)c(CNC[C@@H]6CCC(=O)N6)cnc5c4)c3C)c2C)ccc1CNC[C@@H]1CCC(=O)N1]

### 8-(2-Methyl-3-(4,4,5,5-tetramethyl-1,3,2-dioxaborolan-2-yl)phenyl)-4-oxo-4H-pyrido[1,2-a]pyrimidine-3-carbaldehyde

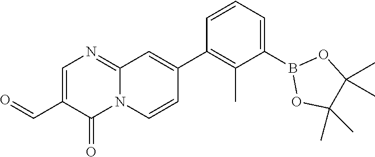 [SMILES: Cc1c(B2OC(C)(C)C(C)(C)O2)cccc1-c1ccn2c(=O)c(C=O)cnc2c1]

To a solution of 8-(3-bromo-2-methylphenyl)-4-oxo-4H-pyrido[1,2-a]pyrimidine-3-carbaldehyde (5 g, 14.6 mmol) and bis (pinacolato) diboron (7.6 g, 29.7 mmol) in dioxane (40 mL) and THE (40 mL) under nitrogen gas were added 1,1′-bis(diphenylphosphino)ferrocene-palladium(II)dichloride dichloromethane complex (1.2 g, 1.5 mmol) and potassium acetate (4 g, 41 mmol). The mixture was stirred at 80° C. for 12 hrs. The mixture was filtered through CELITE® and the filter cake was washed with THE (3×10 mL). The filtrate was concentrated. The residue was purified by normal phase flash SiO₂ chromatography (0-50% ethyl acetate/30% dichloromethane in petroleum ether) to give 8-(2-methyl-3-(4,4,5,5-tetramethyl-1,3,2-dioxaborolan-2-yl)phenyl)-4-oxo-4H-pyrido[1,2-a]pyrimidine-3-carbaldehyde (3 g, 53% yield) as a yellow solid. ¹H NMR (400 MHz, DMSO-d₆) δ ppm 10.19 (s, 1H), 9.22 (d, J=7.2 Hz, 1H), 8.77 (s, 1H), 7.85 (d, J=1.6 Hz, 1H), 7.78 (d, J=6.0 Hz, 1H), 7.67-7.65 (m, 1H), 7.50-7.41 (m, 1H), 7.40-7.35 (m, 1H), 2.5 (s, 3H), 1.33 (s, 12H).

### 8-(3′-Bromo-2,2′-dimethyl-[1,1′-biphenyl]-3-yl)-4-oxo-4H-pyrido[1,2-a]pyrimidine-3-carbaldehyde

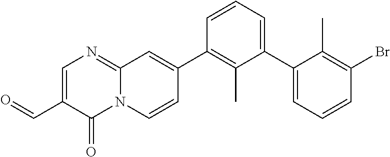 [SMILES: Cc1c(Br)cccc1-c1cccc(-c2ccn3c(=O)c(C=O)cnc3c2)c1C]

To a mixture of 8-(2-methyl-3-(4,4,5,5-tetramethyl-1,3,2-dioxaborolan-2-yl)phenyl)-4-oxo-4H-pyrido[1,2-a]pyrimidine-3-carbaldehyd (6 g, 15.4 mmol) and 1,3-dibromo-2-methylbenzene (9.6 g, 38.4 mmol) in THF/H₂O (5:1, 60 mL) was added [1,1-Bis(diphenylphosphino)ferrocene]palladium(II)chloride dichloromethane complex (1.3 g, 1.5 mmol) and potassium carbonate (6.3 g, 46.1 mmol). The mixture was stirred at 80° C. for 5 hours under N₂. The mixture was concentrated to give the residue. H₂O (50 mL) was added and the mixture was extracted with EtOAc (3×80 mL). The combined organic phase was dried over anhydrous sulfate, filtered and concentrated to give the residue. The residue was purified by normal phase SiO₂ chromatography (0-60% EtOAc/petroleum ether) to afford 8-[3-(3-bromo-2-methyl-phenyl)-2-methyl-phenyl]-4-oxo-pyrido[1,2-a]pyrimidine-3-carbaldehyde (5 g, 62% yield) as a yellow solid. ¹H NMR (400 MHz, DMSO-d₆): δ 10.20 (s, 1H), 9.25 (d, J=7.2 Hz, 1H), 8.79 (s, 1H), 7.96 (d, J=1.2 Hz, 1H), 7.64 (d, J=8 Hz, 1H), 7.47 (t, J=2.4, 2H), 7.29 (s, 2H), 7.25-7.20 (m, 2H), 2.14 (s, 3H), 2.00 (s, 3H).

### 8-(2,2′-Dimethyl-3′-(4,4,5,5-tetramethyl-1,3,2-dioxaborolan-2-yl)-[1,1′-biphenyl]-3-yl)-4-oxo-4H-pyrido[1,2-a]pyrimidine-3-carbaldehyde

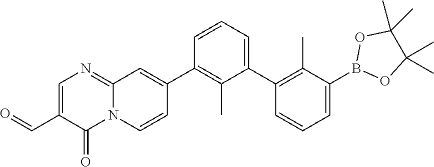 [SMILES: Cc1c(B2OC(C)(C)C(C)(C)O2)cccc1-c1cccc(-c2ccn3c(=O)c(C=O)cnc3c2)c1C]

A mixture of 8-[3-(3-bromo-2-methyl-phenyl)-2-methyl-phenyl]-4-oxo-pyrido[1,2-a]pyrimidine-3-carbaldehyde (5 g, 11.5 mmol) and 4,4,4′,4′,5,5,5′,5′-octamethyl-2,2′-bi(1,3,2-dioxaborolane) (8.8 g, 34.6 mmol) in dioxane (40 mL) was added potassium acetate (3.4 g, 34.6 mmol) and [1,1-bis(diphenylphosphino)ferrocene]palladium(II)chloride dichloromethane complex (942 mg, 1.2 mmol). Then the mixture was stirred at 110° C. for 12 hours under N₂. The mixture was concentrated. The residue was purified by normal phase SiO₂ chromatography (0-70% EtOAc/petroleum ether) to afford 8-(2,2′-dimethyl-3′-(4,4,5,5-tetramethyl-1,3,2-dioxaborolan-2-yl)-[1,1′-biphenyl]-3-yl)-4-oxo-4H-pyrido[1,2-a]pyrimidine-3-carbaldehyde (4 g, 58% yield) as a yellow solid. ¹H NMR (400 MHz, CDCl₃): δ 10.34 (s, 1H), 9.23 (d, J=7.2 Hz, 1H), 8.87 (s, 1H), 7.77 (s, 1H), 7.72 (d, J=7.2 Hz, 1H), 7.39 (d, J=6.8, 1H), 7.33 (t, J=3.8 Hz, 1H), 7.23 (d, J=7.2, 1H), 7.18 (d, J=7.2, 1H), 7.13 (d, J=7.2 Hz, 1H), 2.20 (s, 3H), 1.95 (s, 3H), 1.30 (s, 12H).

### 8-(3′-(5-Formyl-6-methoxypyridin-2-yl)-2,2′-dimethyl-[1,1′-biphenyl]-3-yl)-4-oxo-4H-pyrido[1,2-a]pyrimidine-3-carbaldehyde

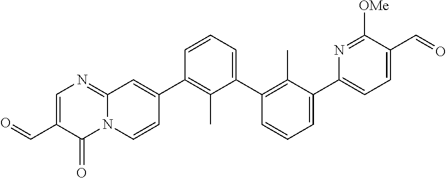 [SMILES: COc1nc(-c2cccc(-c3cccc(-c4ccn5c(=O)c(C=O)cnc5c4)c3C)c2C)ccc1C=O]

A mixture of 8-(2,2′-dimethyl-3′-(4,4,5,5-tetramethyl-1,3,2-dioxaborolan-2-yl)-[1,1′-biphenyl]-3-yl)-4-oxo-4H-pyrido[1,2-a]pyrimidine-3-carbaldehyde (300 mg, 0.62 mmol), 6-chloro-2-methoxynicotinaldehyde (107 mg, 0.62 mmol) in toluene/EtOH/H₂O (8:2:1, 11 mL) were added sodium carbonate (199 mg, 1.87 mmol) and [1,1-Bis(diphenylphosphino)ferrocene]palladium(II) chloride dichloromethane complex (51 mg, 62.5 umol). Then the mixture was stirred at 70° C. for 12 hours under N₂. The mixture was combined with other two batches at the same scale and concentrated. The residue was purified by normal phase SiO₂ chromatography (0-25% EtOAc/petroleum ether) to afford 8-(3′-(5-formyl-6-methoxypyridin-2-yl)-2,2′-dimethyl-[1,1′-biphenyl]-3-yl)-4-oxo-4H-pyrido[1,2-a]pyrimidine-3-carbaldehyde (0.3 g, 32% yield) as a yellow solid. ¹H NMR (400 MHz, DMSO-d₆): δ 10.30 (s, 1H), 10.20 (s, 1H), 9.25 (d, J=7.2, 1H), 8.79 (s, 1H), 8.20 (d, J=8 Hz, 1H), 7.96 (s, 1H), 7.77 (d, J=2 Hz, 1H), 7.53-7.38 (m, 7H), 4.04 (s, 3H), 2.12 (s, 3H), 2.05 (s, 3H).

### 8-(3′-(6-Methoxy-5-(((((S)-5-oxopyrrolidin-2-yl)methyl)amino)methyl)pyridin-2-yl)-2,2′-dimethyl-[1,1′-biphenyl]-3-yl)-3-(((((S)-5-oxopyrrolidin-2-yl)methyl)amino)methyl)-4H-pyrido[1,2-a]pyrimidin-4-one

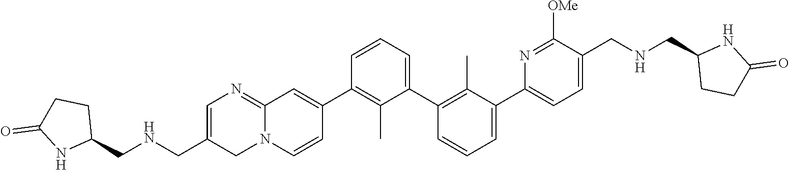 [SMILES: COc1nc(-c2cccc(-c3cccc(C4=CC5=NC=C(CNC[C@@H]6CCC(=O)N6)CN5C=C4)c3C)c2C)ccc1CNC[C@@H]1CCC(=O)N1]

To a solution of 8-(3′-(5-formyl-6-methoxypyridin-2-yl)-2,2′-dimethyl-[1,1′-biphenyl]-3-yl)-4-oxo-4H-pyrido[1,2-a]pyrimidine-3-carbaldehyde (300 mg, 0.6 mmol) in THF/MeOH (1:1, 10 mL) was added (5S)-5-(aminomethyl)pyrrolidin-2-one (738 mg, 4.9 mmol), acetic acid (35 uL, 0.6 mmol) and 4 Å molecular sieve (20 mg). The resultant mixture was stirred at 20° C. for 12 hours under N₂. Then sodium cyanoborohydride (154 mg, 2.4 mmol) was added and the mixture was stirred at 20° C. for 0.5 hours under N₂. The mixture was filtered and the filtrate was concentrated to give the residue. The residue was purified by reverse phase HPLC to afford 8-(3′-(6-methoxy-5-(((((S)-5-oxopyrrolidin-2-yl)methyl)amino) methyl)pyridin-2-yl)-2,2′-dimethyl-[1,1′-biphenyl]-3-yl)-3-(((((S)-5-oxopyrrolidin-2-yl)methyl)amino)methyl)-4H-pyrido[1,2-a]pyrimidin-4-one (42.02 mg) as a white solid. m/z: 686 [M+H]⁺ observed. ¹H NMR (400 MHz, CD₃OD): δ9.13 (d, J=7.2 Hz, 1H), 8.40 (s, 1H), 7.73 (d, J=7.6, 1H), 7.65 (d, J=1.2, 1H), 7.46-7.29 (m, 6H), 7.19 (d, J=7.2, 1H), 7.08 (d, J=7.6, 1H), 3.99 (s, 3H), 3.84 (dd, J₁=4 Hz, J₂=14.8 Hz, 6H), 2.78-2.67 (m, 4H), 2.36-2.25 (m, 6H), 2.10 (s, 3H), 2.08 (s, 3H), 1.87-1.78 (m, 2H).

### Example 32: 8-(2′-Chloro-3′-(6-methoxy-5-(((((S)-5-oxopyrrolidin-2-yl)methyl)amino)methyl)pyridin-2-yl)-2-methyl-[1,1′-biphenyl]-3-yl)-3-(((((S)-5-oxopyrrolidin-2-yl)methyl)amino)methyl)-4H-pyrido[1,2-a]pyrimidin-4-one

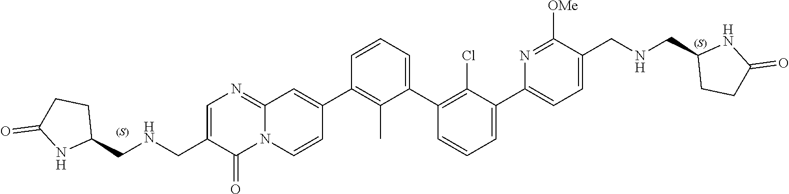

### 8-(3′-Bromo-2′-chloro-2-methyl-[1,1′-biphenyl]-3-yl)-4-oxo-4H1-pyrido[1,2-a]pyrimidine-3-carbaldehyde

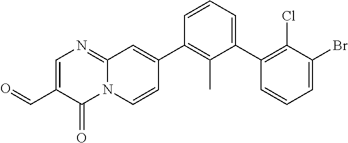 [SMILES: Cc1c(-c2ccn3c(=O)c(C=O)cnc3c2)cccc1-c1cccc(Br)c1Cl]

To a solution of 8-(2-methyl-3-(4,4,5,5-tetramethyl-1,3,2-dioxaborolan-2-yl)phenyl)-4-oxo-4H-pyrido[1,2-a]pyrimidine-3-carbaldehyde (2.5 g, 6.4 mmol) and 1,3-dibromo-2-chlorobenzene (3 g, 11 mmol) in THE (45 mL) and H₂O (15 mL) under nitrogen gas was added 1,1′-bis(diphenylphosphino)ferrocene-palladium(ii) dichloride dichloromethane complex (0.6 g, 0.7 mmol) and potassium carbonate (2.7 g, 19.5 mmol). The mixture was stirred at 80° C. for 1.5 hrs. The mixture was filtered through CELITE® and the filter cake was washed with THE (3×40 mL). The filtrate was washed with saturated aqueous brine solution (2×30 mL) and concentrated. The crude product was triturated with petroleum/ethyl acetate (3/1, 15 mL) at r.t. for 20 min. The yellow solid was collected by filtration to give the product (3.7 g, crude). The mother liquid was purified by normal phase flash SiO₂ chromatography (0˜40% 50% of THE in ethyl acetate/petroleum ether) to give 0.3 g of product as a yellow solid. Totally 8-(3′-bromo-2′-chloro-2-methyl-[1,1′-biphenyl]-3-yl)-4-oxo-4H-pyrido[1,2-a]pyrimidine-3-carbaldehyde (4.0 g, crude) was obtained as a yellow solid. ¹H NMR (400 MHz, DMSO-d₆) δ ppm 10.19 (s, 1H), 9.25 (d, J=7.2 Hz, 1H), 8.79 (s, 1H), 7.93 (m, 1H), 7.90 (m, 1H), 7.81 (d, J=8.0 Hz, 1H), 7.60-7.48 (m, 2H), 7.47-7.35 (m, 1H), 7.28-7.20 (m, 1H), 2.5 (s, 3H).

### 8-(2′-Chloro-2-methyl-3′-(4,4,5,5-tetramethyl-1,3,2-dioxaborolan-2-yl)-[1,1′-biphenyl]-3-yl)-4-oxo-4H-pyrido[1,2-a]pyrimidine-3-carbaldehyde

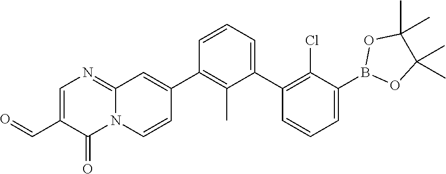 [SMILES: Cc1c(-c2ccn3c(=O)c(C=O)cnc3c2)cccc1-c1cccc(B2OC(C)(C)C(C)(C)O2)c1Cl]

To a solution of 8-(3′-bromo-2′-chloro-2-methyl-[1,1′-biphenyl]-3-yl)-4-oxo-4H-pyrido[1,2-a]pyrimidine-3-carbaldehyde (3.7 g, 8.2 mmol) and bis (pinacolato) diboron (6.5 g, 25.6 mmol) in dioxane (100 mL) under nitrogen gas was added potassium carbonate (3 g, 30.6 mmol) and 1,1′-bis(diphenylphosphino) ferrocene-palladium(ii) dichloride (1 g, 1.4 mmol). The mixture was stirred at 110° C. for 1 hr. The mixture was concentrated. The residue was purified by normal phase flash SiO₂ chromatography (0˜30% 50% of THE in ethyl acetate/petroleum ether) to give 3 g of semi-purified product as a yellow solid. The solid was triturated with petroleum ether/ethyl acetate (3/1, 12 mL) at r.t. for 15 min. The resulting yellow solid was collected by filtration to afford 8-(2′-chloro-2-methyl-3′-(4,4,5,5-tetramethyl-1,3,2-dioxaborolan-2-yl)-[1,1′-biphenyl]-3-yl)-4-oxo-4H-pyrido[1,2-a]pyrimidine-3-carbaldehyde (1.3 g, 31% yield) as a yellow solid. ¹H NMR (400 MHz, DMSO-d₆) δ ppm 10.20 (s, 1H) 9.24 (d, J=7.2 Hz, 1H), 8.79 (s, 1H), 7.93 (s, 1H), 7.73-7.70 (m, 1H), 7.70-7.65 (m, 1H), 7.50-7.41 (m, 4H), 7.35-7.27 (m, 1H), 2.02 (s, 3H), 1.32 (s, 12H).

### 8-(2′-Chloro-3′-(5-formyl-6-methoxypyridin-2-yl)-2-methyl-[1,1′-biphenyl]-3-yl)-4-oxo-4H-pyrido[1,2-a]pyrimidine-3-carbaldehyde

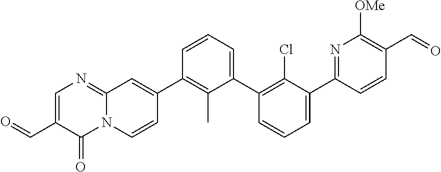 [SMILES: COc1nc(-c2cccc(-c3cccc(-c4ccn5c(=O)c(C=O)cnc5c4)c3C)c2Cl)ccc1C=O]

To a solution of 8-(2′-chloro-2-methyl-3′-(4,4,5,5-tetramethyl-1,3,2-dioxa borolan-2-yl)-[1,1′-biphenyl]-3-yl)-4-oxo-4H-pyrido[1,2-a]pyrimidine-3-carbaldehyde (0.4 g, 0.8 mmol) and 6-bromo-2-methoxynicotinaldehyde (0.22 g, 1 mmol) in dioxane (20 mL) and H₂O (6 mL) under nitrogen gas was added 1,1′-bis(diphenylphosphino)ferrocene-palladium(ii) dichloride (0.1 g, 0.14 mmol) and potassium carbonate (0.33 g, 2.4 mmol). The mixture was stirred at 90° C. for 1.5 hrs. The mixture was extracted with THE (50 mL), washed with saturated aqueous brine solution (2×15 mL), dried over anhydrous sodium sulfate, filtered and concentrated under reduced pressure. The residue was purified by normal phase flash SiO₂ chromatography (0˜60% 25% of ethyl acetate in THF/Petroleum ether) to afford 8-(2′-chloro-3′-(5-formyl-6-methoxypyridin-2-yl)-2-methyl-[1,1′-biphenyl]-3-yl)-4-oxo-4H-pyrido[1,2-a]pyrimidine-3-carbaldehyde (0.25 g, 31% yield) as a yellow solid. MS: m/z found 510 [M+H]⁺; ¹H NMR (400 MHz, DMSO-d₆): δ ppm 10.30 (s, 1H), 10.20 (s, 1H), 9.26 (d, J=7.2 Hz, 1H), 8.79 (s, 1H), 8.21 (d, J=7.6 Hz, 1H), 7.92 (s, 1H), 7.79-7.67 (m, 2H), 7.65-7.59 (m, 1H), 7.56-7.48 (m, 4H), 7.43-7.39 (m, 1H), 4.06 (s, 3H), 2.10 (s, 3H).

### 8-(2′-Chloro-3′-(6-methoxy-5-(((((S)-5-oxopyrrolidin-2-yl)methyl)amino)methyl)pyridin-2-yl)-2-methyl-[1,1′-biphenyl]-3-yl)-3-(((((S)-5-oxopyrrolidin-2-yl)methyl)amino)methyl)-4H-pyrido[1,2-a]pyrimidin-4-one

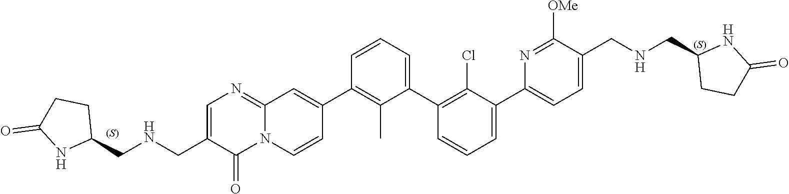

To a mixture of 8-(2′-chloro-3′-(5-formyl-6-methoxypyridin-2-yl)-2-methyl-[1,1′-biphenyl]-3-yl)-4-oxo-4H-pyrido[1,2-a]pyrimidine-3-carbaldehyde (0.25 g, 0.49 mmol) and (S)-5-(aminomethyl)pyrrolidin-2-one as a hydrochloride (0.6 g, 4.0 mmol) in dichloromethane (30 mL) was added triethylamine (1 mL, 7.2 mmol) and 4 Å molecular sieve (1 g). The mixture was stirred at r.t. for 15 hrs. Sodium cyanoborohydride (0.3 g, 4.8 mmol) was added and the mixture was stirred at r.t. for 5 hrs. The reaction was filtered through CELITE® and the filter cake was washed with THE (2×10 mL). The filtrate was washed with saturated aqueous brine solution (3×10 mL) and concentrated. The residue was purified by reverse phase HPLC (neutral conditions) to give 0.2 g of the product as a white solid. The solid contained a large amount of TEA. The solid was further purified by reverse phase HPLC (hydrochloric acid conditions) to give 65 mg of the product as a hydrochloride salt, but about 8% of de-methylation byproduct was detected by LCMS. The mixture was further purified by reverse phase HPLC (neutral conditions) to afford 8-(2′-chloro-3′-(6-methoxy-5-(((((S)-5-oxopyrrolidin-2-yl)methyl)amino)methyl)pyridin-2-yl)-2-methyl-[1,1′-biphenyl]-3-yl)-3-(((((S)-5-oxopyrrolidin-2-yl)methyl)amino)methyl)-4H-pyrido[1,2-a]pyrimidin-4-one (38.5 mg) as a white solid. MS: m/z found 706 [M+H]⁺. ¹H NMR (400 MHz, CD₃OD): δ9.13 (d, J=7.6 Hz, 1H), 8.40 (s, 1H), 7.73 (d, J=7.2 Hz, 1H), 7.68-7.58 (m, 2H), 7.54-7.40 (m, 4H), 7.39-7.29 (m, 2H), 7.24 (d, J=7.6 Hz, 1H), 4.02 (s, 3H), 3.93-3.76 (m, 6H), 2.79-2.62 (m, 4H), 2.42-2.22 (m, 6H), 2.13 (s, 3H), 1.90-1.75 (m, 2H).

### Example 33: 8-(2,2′-Dichloro-3′-(6-methoxy-5-(((((S)-5-oxopyrrolidin-2-yl)methyl)amino)methyl)pyridin-2-yl)-[1,1′-biphenyl]-3-yl)-3-(((((S)-5-oxopyrrolidin-2-yl)methyl)amino)methyl)-4H-pyrido[1,2-a]pyrimidin-4-one

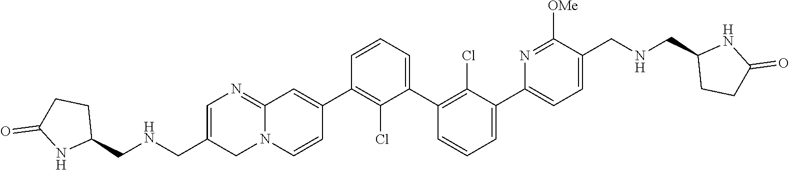 [SMILES: COc1nc(-c2cccc(-c3cccc(C4=CC5=NC=C(CNC[C@@H]6CCC(=O)N6)CN5C=C4)c3Cl)c2Cl)ccc1CNC[C@@H]1CCC(=O)N1]

### 8-(2,2′-Dichloro-3′-(5-formyl-6-methoxypyridin-2-yl)-[11,′-biphenyl]-3-yl)-4-oxo-4H-pyrido[1,2-a]pyrimidine-3-carbaldehyde

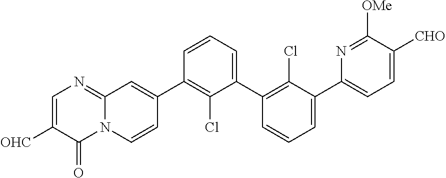 [SMILES: COc1nc(-c2cccc(-c3cccc(-c4ccn5c(=O)c(C=O)cnc5c4)c3Cl)c2Cl)ccc1C=O]

To a mixture of 8-[2-chloro-3-(4,4,5,5-tetramethyl-1,3,2-dioxaborolan-2-yl)phenyl]-4-oxo-pyrido[1,2-a]pyrimidine-3-carbaldehyde (500 mg, 1.2 mmol) and 6-(3-bromo-2-chloro phenyl)-2-methoxynicotinaldehyde (397 mg, 1.2 mmol) in methylbenzene/EtOH/H₂O mixture (8:2:1, 27.5 mL) was added sodium carbonate (387 mg, 3.6 mmol) and [1,1-Bis(diphenylphosphino)ferrocene]palladium(II)chloride dichloromethane complex (99 mg, 0.12 mmol). Then the mixture was stirred at 70° C. for 12 h under N₂. The mixture was concentrated to give the residue. The residue was purified by normal phase SiO₂ chromatography (0-80% EtOAc/petroleum ether) to afford 8-(2,2′-dichloro-3′-(5-formyl-6-methoxypyridin-2-yl)-[1,1′-biphenyl]-3-yl)-4-oxo-4H-pyrido[1,2-a]pyrimidine-3-carbaldehyde (400 mg, 62% yield) as a yellow solid. ¹H NMR (400 MHz, DMSO-d₆): δ 10.31 (s, 1H), 10.21 (s, 1H), 9.29 (d, J=7.2 Hz, 1H), 8.80 (s, 1H), 8.23 (d, J=8 Hz, 1H), 8.06 (d, J=2 Hz, 1H), 7.79-7.51 (m, 7H), 6.88 (s, 1H), 4.07 (s, 3H).

### 8-(2,2′-Dichloro-3′-(6-methoxy-5-(((((S)-5-oxopyrrolidin-2-yl)methyl)amino)methyl)pyridin-2-yl)-[1,1′-biphenyl]-3-yl)-3-(((((S)-5-oxopyrrolidin-2-yl)methyl)amino)methyl)-4H-pyrido[1,2-a]pyrimidin-4-one

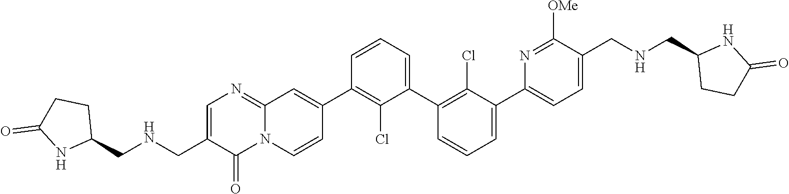 [SMILES: COc1nc(-c2cccc(-c3cccc(-c4ccn5c(=O)c(CNC[C@@H]6CCC(=O)N6)cnc5c4)c3Cl)c2Cl)ccc1CNC[C@@H]1CCC(=O)N1]

A mixture of 8-[2-chloro-3-[2-chloro-3-(5-formyl-6-methoxy-2-pyridyl)phenyl]phenyl]-4-oxo-pyrido[1,2-a]pyrimidine-3-carbaldehyde (0.39 g, 0.73 mmol), (5S)-5-(aminomethyl)pyrrolidin-2-one (1.1 g, 7.3 mmol, HCl), 4 Å molecular sieve (0.05 g) and sodium acetate (0.36 g, 4.41 mmol) in DCM (30 mL) was stirred at room temperature for 12 h under N₂. Sodium triacetoxyhydroborate (3.1 g, 14.7 mmol) was then added to the mixture and was stirred at room temperature for 0.5 h under N₂. The mixture was filtered to give the filtrate. The filtrate was concentrated to give the residue. The residue was purified by prep-HPLC (column: Kromasil C18 (250*50 mm*10 um); mobile phase: [water (10 mM NH₄HCO₃)-ACN]; B %: 25%-50%, 10 min) to afford 8-(2,2′-dichloro-3′-(6-methoxy-5-(((((S)-5-oxopyrrolidin-2-yl)methyl)amino)methyl)pyridin-2-yl)-[1,1′-biphenyl]-3-yl)-3-(((((S)-5-oxopyrrolidin-2-yl)methyl)amino)methyl)-4H-pyrido[1,2-a]pyrimidin-4-one (57.56 mg) as a white solid. m/z: 726 [M+H]⁺ observed. ¹H NMR (400 MHz, CD₃OD): δ9.12 (d, J=7.6 Hz, 1H), 8.40 (s, 1H), 7.76-7.71 (m, 2H), 7.66-7.53 (m, 3H), 7.51-7.48-7.29 (m, 3H), 7.38 (d, J=7.6, 1H), 7.23 (d, J=7.6, 1H), 4.01 (s, 3H), 3.86-3.82 (m, 6H), 2.75-2.66 (m, 4H), 2.35-2.24 (m, 6H), 1.83-1.79 (m, 2H).

### Example 34: 2,2′-(((2,2′-Dichloro-[1,1′-biphenyl]-3,3′-diyl)bis(1-methyl-1,2,3,4-tetrahydro-1,8-naphthyridine-7,4-diyl))bis(azanediyl))bis(ethan-1-ol)

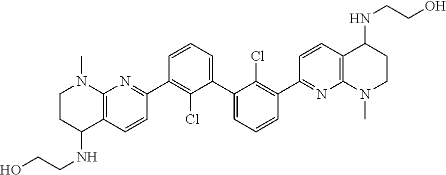 [SMILES: CN1CCC(NCCO)c2ccc(-c3cccc(-c4cccc(-c5ccc6c(n5)N(C)CCC6NCCO)c4Cl)c3Cl)nc21]

### 3-((6-Bromopyridin-2-yl)-(methyl)-amino)-propanoic acid

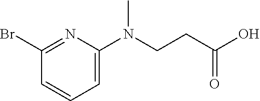

6-Bromo-N-methyl-pyridin-2-amine (1.50 g, 8.02 mmol) and acrylic acid (1.16 g, 16.04 mmol) were heated neat in a sealed tube at 135° C. for 20 minutes. The reaction mixture was then cooled to rt, diluted with 30 ml DCM, and extracted with aqueous 1M NaOH solution (3×15 ml). The basic aqueous solution, containing desired product, was subsequently acidified to pH=4 by slow addition of aqueous 1M HCl solution with stirring. The resulting acidic aqueous solution was then extracted with DCM (3×20 ml), and the combined organics were washed with brine (30 ml), dried over sodium sulfate, filtered and concentrated to afford 3-[(6-bromo-2-pyridyl)-methyl-amino]propanoic acid (1250.00 mg, 60.2% yield) as a light brown oil. MS: m/z found 259, 261 [M+H]⁺.

### 7-Bromo-1-methyl-2,3-dihydro-1,8-naphthyridin-4(1H)-one

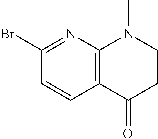

3-[(6-bromo-2-pyridyl)-methyl-amino]propanoic acid (1500.00 mg, 5.79 mmol) and polyphosphoric acid (2.75 mL, 5.67 g, 57.89 mmol) were mixed and the viscous sample was heated neat with stirring at 120° C. for 25 minutes. The reaction was cooled to rt and to the residue was slowly added 50 ml cold water with stirring. The resulting solution was extracted with EtOAc (3×30 ml) and the combined organics were washed with brine (20 ml), dried over sodium sulfate, then filtered and concentrated. Crude sample was purified by silica gel chromatography, eluting with 5-40% EtOAc gradient in hexane to afford 7-bromo-1-methyl-2,3-dihydro-1,8-naphthyridin-4-one (208.00 mg, 14.9% yield) as a tan solid. MS: m/z found 241, 243 [M+H]⁺. ¹H NMR (400 MHz, Chloroform-d) δ 7.84 (dd, J=7.9, 0.8 Hz, 1H), 6.78 (dd, J=7.9, 0.8 Hz, 1H), 3.55 (t, J=7.1 Hz, 2H), 3.19 (d, J=0.8 Hz, 3H), 2.92-2.50 (m, 2H).

### 2-((7-Bromo-1-methyl-1,2,3,4-tetrahydro-1,8-naphthyridin-4-yl)-amino)-ethan-1-ol

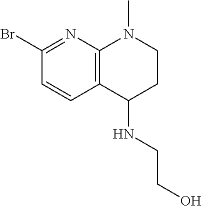 [SMILES: CN1CCC(NCCO)c2ccc(Br)nc21]

7-Bromo-1-methyl-2,3-dihydro-1,8-naphthyridin-4-one (24 mg, 0.10 mmol), 2-aminoethanol (11.91 mg, 0.19 mmol) and tetraethoxytitanium (66 mg, 0.29 mmol) were suspended in 2 ml anhydrous THE and the reaction mixture was heated at 105° C. for 1 hour in a sealed tube. The reaction was then cooled to −50° C., and NaBH₄ (11 mg, 0.29 mmol) was added portionwise under a nitrogen stream. The mixture was gradually warmed to rt over 1 hour.

The reaction was then slowly added to an ice water solution (10 ml) and 8 ml EtOAc was also added with stirring. The resulting slurry was filtered through CELITE® and the layers were partitioned. The EOAc layer was further washed with brine, dried over sodium sulfate, and concentrated to afford 2-[(7-bromo-1-methyl-3,4-dihydro-2H-1,8-naphthyridin-4-yl)amino]ethanol (27 mg) as a tan solid. Material was used as—is for the next step. MS: m/z found 286, 288.05 [M+H]⁺.

### 2,2′-(((2,2′-Dichloro-[1,1′-biphenyl]-3,3′-diyl)bis(1-methyl-1,2,3,4-tetrahydro-1,8-naphthyridine-7,4-diyl))bis(azanediyl))bis(ethan-1-ol)

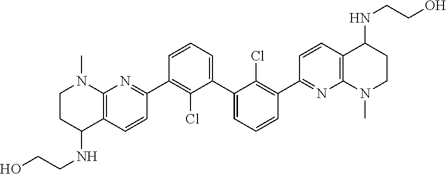 [SMILES: CN1CCC(NCCO)c2ccc(-c3cccc(-c4cccc(-c5ccc6c(n5)N(C)CCC6NCCO)c4Cl)c3Cl)nc21]

Tetrakis(triphenylphosphine)palladium(0) (8 mg, 0.01 mmol), potassium carbonate (20 mg, 0.14 mmol), 2-[2-chloro-3-[2-chloro-3-(4,4,5,5-tetramethyl-1,3,2-dioxaborolan-2-yl)phenyl]phenyl]-4,4,5,5-tetramethyl-1,3,2-dioxaborolane (17 mg, 0.04 mmol), and 2-[(7-bromo-1-methyl-3,4-dihydro-2H-1,8-naphthyridin-4-yl)amino]ethanol (26 mg, 0.09 mmol) were suspended in 2 ml of 4:1 (v v) dioxane/water, and the solution was heated at 110° C. for 30 minutes. The reaction mixture was cooled to rt, filtered through CELITE®, and the filtrate was concentrated. The crude product was purified by reverse phase HPLC to afford 2-[[7-[2-chloro-3-[2-chloro-3-[5-(2-hydroxyethylamino)-8-methyl-6,7-dihydro-5H-1,8-naphthyridin-2-yl]phenyl]phenyl]-1-methyl-3,4-dihydro-2H-1,8-naphthyridin-4-yl]amino]ethanol (12.10 mg, 53.4% yield) as a white foam MS: m/z found 633.5, 635 [M+H]⁺. ¹H NMR (400 MHz, Chloroform-d) δ 7.68-7.59 (m, 2H), 7.41-7.33 (m, 4H), 7.29 (p, J=2.1 Hz, 2H), 6.93-6.81 (m, 2H), 3.79 (s, 2H), 3.68 (dd, J=7.5, 3.1 Hz, 4H), 3.51 (d, J=10.9 Hz, 2H), 3.38-3.26 (m, 2H), 3.20-3.12 (m, 6H), 2.91 (d, J=5.5 Hz, 4H), 2.07-1.94 (m, 4H).

### Example 35: 2,2′-(2,2′-Dichloro-[1,1′-biphenyl]-3,3′-diyl)bis(9-methyl-7-(3-(methylamino)propoxy)-4H-pyrido[1,2-a]pyrimidin-4-one)

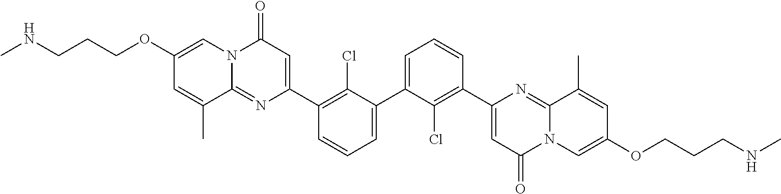

### 7-Fluoro-2-hydroxy-9-methyl-4H-pyrido[1,2-a]pyrimidin-4-one

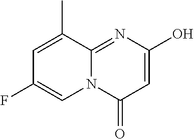

To a stirring solution of 5-fluoro-3-methyl-pyridin-2-amine (1.00 g, 7.93 mmol) at 0° C. in chloroform (16 mL) was added malonyl chloride (1.54 mL, 15.9 mmol) dropwise via syringe. The reaction was warmed to ambient temperature while stirring. After 1 h of stirring at rt, the reaction was heated to 50° C. while stirring overnight. The solid was filtered, rinsed with DCM (30 mL), and dried in a vacuum oven to afford 7-fluoro-2-hydroxy-9-methyl-4H-pyrido[1,2-a]pyrimidin-4-one (0.46 g) as a yellow solid. Due to solubility issues the crude material was used in the following reactions without further purification. MS: m/z found 195.1 [M+H]⁺. ¹H NMR (400 MHz, DMSO-d₆) δ 9.03-8.89 (m, 1H), 8.08 (ddt, J=7.3, 3.0, 1.0 Hz, 1H), 3.30 (dq, J=3.1, 1.5 Hz, 3H), 2.67-2.54 (m, 2H). ¹⁹F NMR (376 MHz, DMSO-d₆) δ-134.42 (dd, J=7.6, 4.2 Hz).

### 2-Chloro-7-fluoro-9-methyl-4H-pyrido[1,2-a]pyrimidin-4-one

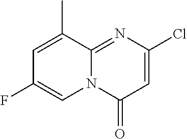

To a vial charged with light yellow solid 7-fluoro-2-hydroxy-9-methyl-4H-pyrido[1,2-a]pyrimidin-4-one (2.18 g, 11.2 mmol) at rt was added phosphoryl trichloride (10.5 mL, 112.2 mmol) dropwise via syringe. The suspension was moved to a heating block and heated to 110° C. After 16 h reaction time, the excess phosphoryl trichloride was removed under reduced pressure. The concentrated crude mixture was dissolved in minimal DCM and transferred slowly to a vigorously stirring ice/water bath. To the orange/red solution was added sat. aq. NaHCO₃ solution slowly, allowing for off gassing until the pH was adjusted to pH=7. The solution was diluted with DCM (50 mL) and extracted. The aqueous phase was extracted with DCM (3×30 mL), the combined organic phases were dried over anhydrous sodium sulfate, filtered, and concentrated.

The crude solid was purified by silica flash column chromatography, 0-50% EA/H to afford 2-chloro-7-fluoro-9-methyl-4H-pyrido[1,2-a]pyrimidin-4-one (1.60 g) as a yellow solid. MS: m/z found 213.1 [M+H]⁺. ¹H NMR (400 MHz, Chloroform-d) δ 8.85 (ddd, J=4.3, 2.8, 0.7 Hz, 1H), 7.63 (ddd, J=7.3, 2.8, 1.1 Hz, 1H), 6.48 (s, 1H), 2.63 (s, 4H). ¹⁹F NMR (376 MHz, Chloroform-d) δ-131.55 (dd, J=7.3, 4.5 Hz).

### 2-(2-Chloro-3-hydroxyphenyl)-7-fluoro-9-methyl-4H-pyrido[1,2-a]pyrimidin-4-one

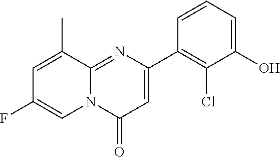 [SMILES: Cc1cc(F)cn2c(=O)cc(-c3cccc(O)c3Cl)nc12]

To a 100 mL reaction flask equipped with magnetic stirbar was added potassium carbonate (1.95 g, 14.11 mmol), 2-chloro-7-fluoro-9-methyl-4H-pyrido[1,2-a]pyrimidin-4-one (1.00 g, 4.70 mmol), and Pd(dppf)Cl₂ (0.38 g, 0.47 mmol), followed by 2-chloro-3-(4,4,5,5-tetramethyl-1,3,2-dioxaborolan-2-yl)phenol (1.44 g, 5.64 mmol) crude solution in dioxane (30 mL). The dark brown suspension was sparged with N₂ for 10 min prior to heating at 80° C. under N₂ while stirring for 16 h. The reaction mixture was cooled to rt and quenched by the addition of water (15 mL) while vigorously stirring. The resulting biphasic mixture was filtered through Celite and the filtrate was extracted with ethyl acetate (3×15 mL). Combined organic layers were dried over anhydrous sodium sulfate, filtered, and concentrated to a dark brown/black oil. The crude oil was suspended in DCM (10 mL) and agitated for 20 min. The solid was filtered carefully and washed with an additional 5 mL of DCM to afford 2-(2-chloro-3-hydroxyphenyl)-7-fluoro-9-methyl-4H-pyrido[1,2-a]pyrimidin-4-one (0.74 g). MS: m/z found 305.1 [M+H]⁺.

### 2-Chloro-3-(7-fluoro-9-methyl-4-oxo-4H-pyrido[1,2-a]pyrimidin-2-yl)phenyl trifluoromethanesulfonate

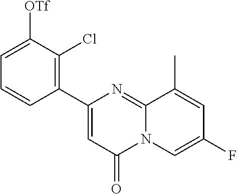 [SMILES: Cc1cc(F)cn2c(=O)cc(-c3cccc(OS(=O)(=O)C(F)(F)F)c3Cl)nc12]

To a stirring suspension of 2-(2-chloro-3-hydroxyphenyl)-7-fluoro-9-methyl-4H-pyrido[1,2-a]pyrimidin-4-one (0.32 g, 1.05 mmol) dissolved in pyridine (0.85 mL, 10.50 mmol) and MeCN (7 mL) at 0° C. was added trifluoromethanesulfonic anhydride (0.25 mL, 1.47 mmol) dropwise over 2 min. After 10 min of stirring, the reaction mixture was diluted by the addition of DCM (10 mL) and quenched by the slow addition of 10% aq HCl solution (2 mL). The biphasic mixture was vigorously stirred for 10 min. The aqueous phase was extracted with DCM (2×10 mL), combined organic phases were washed with sat. aq. NaHCO₃ solution (15 mL), dried over anhydrous sodium sulfate, filtered, and concentrated under reduced pressure. The crude material was purified by silica flash column chromatography from 0-50% EA/H to afford 2-chloro-3-(7-fluoro-9-methyl-4-oxo-4H-pyrido[1,2-a]pyrimidin-2-yl)phenyl trifluoromethanesulfonate (0.33 g) as an orange oil. MS: m/z found 437.1 [M+H]⁺. ¹H NMR (400 MHz, Chloroform-d) δ 8.92 (dd, J=4.6, 2.9 Hz, 1H), 7.71 (dd, J=6.5, 2.9 Hz, 1H), 7.59 (ddt, J=7.3, 2.2, 1.1 Hz, 1H), 7.53-7.44 (m, 2H), 6.79 (s, 1H), 2.65 (d, J=1.0 Hz, 3H). ¹⁹F NMR (376 MHz, Chloroform-d) δ-73.41,-132.39.

### 2-(2-Chloro-3-(4,4,5,5-tetramethyl-1,3,2-dioxaborolan-2-yl)phenyl)-7-fluoro-9-methyl-4H-pyrido[1,2-a]pyrimidin-4-one

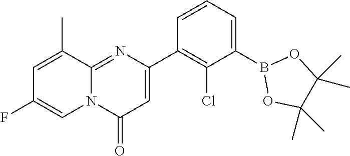 [SMILES: Cc1cc(F)cn2c(=O)cc(-c3cccc(B4OC(C)(C)C(C)(C)O4)c3Cl)nc12]

2-Chloro-3-(7-fluoro-9-methyl-4-oxo-4H-pyrido[1,2-a]pyrimidin-2-yl)phenyl trifluoromethanesulfonate (114 mg, 0.26 mmol), 4,4,5,5-tetramethyl-2-(4,4,5,5-tetramethyl-1,3,2-dioxaborolan-2-yl)-1,3,2-dioxaborolane (73 mg, 0.29 mmol), potassium acetate (71.6 mg, 0.73 mmol), and Pd(dppf)Cl₂ (21 mg, 0.03 mmol) were suspended in dioxane (5 mL). The reaction mixture was sparged with N₂, heated at 80° C. for 8 h under a N₂ atmosphere. The reaction was cooled to ambient temperature, diluted DCM (10 mL) and quenched by the addition of water (10 mL). The aqueous phase was extracted with DCM (2×10 mL), combined organic layers were washed with sat. aq. NaHCO₃ solution (10 mL), dried over anhydrous sodium sulfate, filtered, and concentrated under reduced pressure. The crude material was purified by silica flash column chromatography 0-30% EA/hexanes to afford 2-(2-chloro-3-(4,4,5,5-tetramethyl-1,3,2-dioxaborolan-2-yl)phenyl)-7-fluoro-9-methyl-4H-pyrido[1,2-a]pyrimidin-4-one as a mixture (70 mg) used in the following reaction without further purification. MS: m/z found 415.3 [M+H]⁺.

### 2,2′-(2,2′-Dichloro-[1,1′-biphenyl]-3,3′-diyl)bis(7-fluoro-9-methyl-4H-pyrido[1,2-a]pyrimidin-4-one)

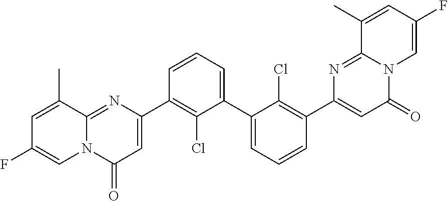 [SMILES: Cc1cc(F)cn2c(=O)cc(-c3cccc(-c4cccc(-c5cc(=O)n6cc(F)cc(C)c6n5)c4Cl)c3Cl)nc12]

2-Chloro-3-(7-fluoro-9-methyl-4-oxo-4H-pyrido[1,2-a]pyrimidin-2-yl)phenyl trifluoromethanesulfonate (33 mg, 0.08 mmol), 2-(2-chloro-3-(4,4,5,5-tetramethyl-1,3,2-dioxaborolan-2-yl)phenyl)-7-fluoro-9-methyl-4H-pyrido[1,2-a]pyrimidin-4-one (34.0 mg, 0.08 mmol), cesium carbonate (62 mg, 0.19 mmol), and Pd(dppf)Cl₂ (6 mg, 0.01 mmol) were suspended in dioxane (5 mL). The reaction was sparged with N₂ for 5 min and then heated at 80° C. for 16 h. The reaction mixture was cooled to rt, filtered over a bed of CELITE®, and rinsed with ethyl acetate (10 mL). The resulting filtrate was washed with water (2×10 mL). The organic phase was dried over anhydrous sodium sulfate, filtered, and concentrated to give a dark brown/black oil. The residue was purified by silica flash column chromatography to afford 2,2′-(2,2′-dichloro-[1,1′-biphenyl]-3,3′-diyl)bis(7-fluoro-9-methyl-4H-pyrido[1,2-a]pyrimidin-4-one (15 mg). MS: m/z found 575.2 [M+H]⁺. ¹H NMR (400 MHz, Chloroform-d) δ8.93 (dd, J=4.5, 2.8 Hz, 2H), 7.73 (dd, J=7.6, 1.8 Hz, 2H), 7.57 (ddd, J=7.5, 2.9, 1.3 Hz, 2H), 7.53-7.45 (m, 2H), 7.42 (dd, J=7.6, 1.8 Hz, 2H), 6.88 (d, J=1.4 Hz, 2H), 2.68 (d, J=1.1 Hz, 6H). ¹⁹F NMR (376 MHz, Chloroform-d) δ-73.37,-133.06 (dd, J=7.4, 4.8 Hz).

### Di-tert-butyl ((((2,2′-dichloro-[1,1′-biphenyl]-3,3′-diyl)bis(9-methyl-4-oxo-4H-pyrido[1,2-a]pyrimidine-2,7-diyl))bis(oxy))bis(propane-3,1-diyl))bis(methylcarbamate)

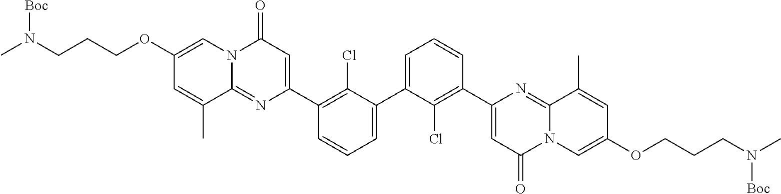

To a stirring suspension of sodium hydride (4 mg, 0.10 mmol) in THE (2.5 mL) at rt under N₂ was slowly added tert-butyl N-(3-hydroxypropyl)-N-methyl-carbamate (16 mg, 0.08 mmol) dropwise via syringe. The resulting suspension was stirred at rt for 15 min prior to the addition of a THE solution (1.5 mL) of 2,2′-(2,2′-dichloro-[1,1′-biphenyl]-3,3′-diyl)bis(7-fluoro-9-methyl-4H-pyrido[1,2-a]pyrimidin-4-one (12.0 mg, 0.02 mmol) dropwise via syringe. The reaction mixture was heated to 70° C. After 3 h of heating, the reaction was cooled to rt, diluted with ethyl acetate (5 mL), and quenched by the slow addition of water (5 mL). The aqueous phase was extracted with EtOAc (2×5 mL), combined organic phases were dried over anhydrous sodium sulfate, filtered, and concentrated. The crude material was purified by silica flash column chromatography 0-50% EA/hexanes to afford di-tert-butyl ((((2,2′-dichloro-[1,1′-biphenyl]-3,3′-diyl)bis(9-methyl-4-oxo-4H-pyrido[1,2-a]pyrimidine-2,7-diyl))bis(oxy))bis(propane-3,1-diyl))bis(methylcarbamate) (12 mg) as a white solid. MS: m/z found 913.4 [M+H]⁺. ¹H NMR (400 MHz, Chloroform-d) δ 8.51 (d, J=2.8 Hz, 2H), 7.74 (dd, J=7.7, 1.8 Hz, 2H), 7.53-7.36 (m, 6H), 6.86 (s, 2H), 4.10 (t, J=6.0 Hz, 4H), 3.44 (s, 4H), 2.90 (s, 6H), 2.64 (s, 6H), 2.14-2.05 (m, 4H), 1.45 (s, 18H).

### 2,2′-(2,2′-Dichloro-[1,1′-biphenyl]-3,3′-diyl)bis(9-methyl-7-(3-(methylamino)propoxy)-4H-pyrido[1,2-a]pyrimidin-4-one)

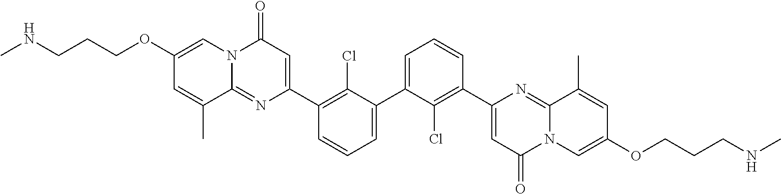

To a reaction vial charged with di-tert-butyl ((((2,2′-dichloro-[1,1′-biphenyl]-3,3′-diyl)bis(9-methyl-4-oxo-4H-pyrido[1,2-a]pyrimidine-2,7-diyl))bis(oxy))bis(propane-3,1-diyl))bis(methylcarbamate) (10.0 mg, 0.01 mmol) at rt was added methanol (2 mL), followed by a 4N dioxane solution of hydrogen chloride (0.14 mL, 0.55 mmol) in a steady stream. After stirring overnight, the methanol was removed under vacuum. The crude material was dissolved in DMSO (1 mL) and purified by HPLC (9 min gradient 15-75% MeCN/H₂O) to afford 2,2′-(2,2′-dichloro-[1,1′-biphenyl]-3,3′-diyl)bis(9-methyl-7-(3-(methylamino)propoxy)-4H-pyrido[1,2-a]pyrimidin-4-one) (5.0 mg). MS: m/z found 713.3 [M+H]⁺. ¹H NMR (400 MHz, Methanol-d₄) δ 8.58 (d, J=2.9 Hz, 2H), 7.80-7.69 (m, 4H), 7.61-7.43 (m, 4H), 6.75 (s, 2H), 4.29 (t, J=5.8 Hz, 4H), 3.26 (s, 4H), 2.78 (s, 6H), 2.66 (d, J=2.9 Hz, 6H), 2.35-2.20 (m, 4H).

### Example 36: 8-(2′-Chloro-3′-(6-methoxy-5-(((((S)-5-oxopyrrolidin-2-yl)methyl)amino)methyl)pyrazin-2-yl)-2-methyl-[1,1′-biphenyl]-3-yl)-3-(((((S)-5-oxopyrrolidin-2-yl)methyl)amino)methyl)-4H-pyrido[1,2-a]pyrimidin-4-one

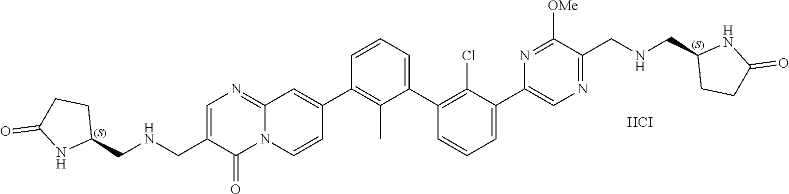

### 8-(2-Methyl-3-(4,4,5,5-tetramethyl-1,3,2-dioxaborolan-2-yl)phenyl)-4-oxo-4H-pyrido[1,2-a]pyrimidine-3-carbaldehyde

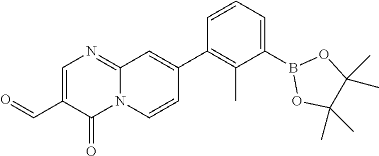 [SMILES: Cc1c(B2OC(C)(C)C(C)(C)O2)cccc1-c1ccn2c(=O)c(C=O)cnc2c1]

To a solution of 8-(3-bromo-2-methylphenyl)-4-oxo-4H-pyrido[1,2-a]pyrimidine-3-carbaldehyde (5 g, 14.6 mmol) and 4,4,4′,4′,5,5,5′,5′-octamethyl-2,2′-bi(1,3,2-dioxaborolane) (7.6 g, 29.7 mmol) in dioxane (40 mL) and THE (40 mL) under N₂ was added 1,1′-bis(diphenylphosphino)ferrocene-palladium(II)dichloride dichloromethane complex (1.2 g, 1.5 mmol) and potassium acetate (4 g, 41 mmol). The mixture was stirred at 80° C. for 12 hrs. The mixture was filtered through CELITE® and the filter cake was washed with THE (3×10 mL). The filtrate was concentrated. The residue was purified by normal phase flash SiO₂ chromatography (0-50% ethyl acetate/30% dichloromethane in petroleum ether) to give 8-(2-methyl-3-(4,4,5,5-tetramethyl-1,3,2-dioxaborolan-2-yl)phenyl)-4-oxo-4H-pyrido[1,2-a]pyrimidine-3-carbaldehyde (3.0 g, 53% yield) as a yellow solid. ¹H NMR (400 MHz, DMSO-d₆) δ ppm 10.19 (s, 1H), 9.22 (d, J=7.2 Hz, 1H), 8.77 (s, 1H), 7.85 (d, J=1.6 Hz, 1H), 7.78 (d, J=6.0 Hz, 1H), 7.67-7.65 (m, 1H), 7.50-7.41 (m, 1H), 7.40-7.35 (m, 1H), 2.5 (s, 3H), 1.33 (s, 12H).

### 8-(3′-Bromo-2′-chloro-2-methyl-[1,1′-biphenyl]-3-yl)-4-oxo-4H-pyrido[1,2-a]pyrimidine-3-carbaldehyde

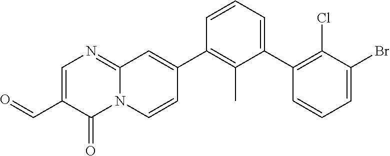 [SMILES: Cc1c(-c2ccn3c(=O)c(C=O)cnc3c2)cccc1-c1cccc(Br)c1Cl]

To a solution of 8-(2-methyl-3-(4,4,5,5-tetramethyl-1,3,2-dioxaborolan-2-yl)phenyl)-4-oxo-4H-pyrido[1,2-a]pyrimidine-3-carbaldehyde (2.5 g, 6.4 mmol) and 1,3-dibromo-2-chlorobenzene (3.0 g, 11 mmol) in THF (45 mL) and H₂O (15 mL) under N₂ was added 1,1′-bis(diphenylphosphino)ferrocene-palladium(ii) dichloride dichloromethane complex (0.6 g, 0.7 mmol) and potassium carbonate (2.7 g, 19.5 mmol). The mixture was stirred at 80° C. for 1.5 hrs. The mixture was filtered through CELITE® and the filter cake was washed with THE (3×40 mL). The filtrate was washed with saturated aqueous brine solution (2×30 mL) and concentrated. The crude product was triturated with petroleum/ethyl acetate (3/1, 15 mL) at r.t. for 20 min. The yellow solid was collected by filtration to give the product (3.7 g, crude). The mother liquid was purified by normal phase flash SiO₂ chromatography (0˜40% 50% of THE in ethyl acetate/petroleum ether) to give 0.3 g of product as a yellow solid. 8-(3′-bromo-2′-chloro-2-methyl-[1,1′-biphenyl]-3-yl)-4-oxo-4H-pyrido[1,2-a]pyrimidine-3-carbaldehyde (4.0 g, crude) was obtained as a yellow solid. ¹H NMR (400 MHz, DMSO-d₆) δ ppm 10.19 (s, 1H), 9.25 (d, J=7.2 Hz, 1H), 8.79 (s, 1H), 7.93 (m, 1H), 7.90 (m, 1H), 7.81 (d, J=8.0 Hz, 1H), 7.60-7.48 (m, 2H), 7.47-7.35 (m, 2H), 7.28-7.20 (m, 1H), 2.5 (s, 3H).

### 8-(2′-Chloro-2-methyl-3′-(4,4,5,5-tetramethyl-1,3,2-dioxaborolan-2-yl)-[1,1′-biphenyl]-3-yl)-4-oxo-4H-pyrido[1,2-a]pyrimidine-3-carbaldehyde

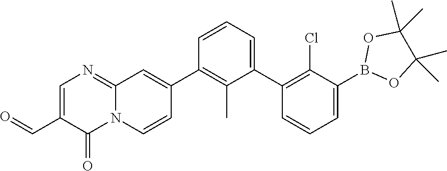 [SMILES: Cc1c(-c2ccn3c(=O)c(C=O)cnc3c2)cccc1-c1cccc(B2OC(C)(C)C(C)(C)O2)c1Cl]

To a solution of 8-(3′-bromo-2′-chloro-2-methyl-[1,1′-biphenyl]-3-yl)-4-oxo-4H-pyrido[1,2-a]pyrimidine-3-carbaldehyde (3.7 g, 8.2 mmol) and 4,4,4′,4′,5,5,5′,5′-octamethyl-2,2′-bi(1,3,2-dioxaborolane) (6.5 g, 25.6 mmol) in dioxane (100 mL) under N₂ was added potassium carbonate (3.0 g, 30.6 mmol) and 1,1′-bis(diphenylphosphino)ferrocene-palladium(ii) dichloride (1.0 g, 1.4 mmol). The mixture was stirred at 110° C. for 1 hr. The mixture was concentrated. The residue was purified by normal phase flash SiO₂ chromatography (0˜30% 50% of THE in ethyl acetate/petroleum ether) to give 3 g of semi-purified product as a yellow solid. The solid was triturated with petroleum ether/ethyl acetate (3/1, 12 mL) at r.t. for 15 min. The resulting yellow solid was collected by filtration to afford 8-(2′-chloro-2-methyl-3′-(4,4,5,5-tetramethyl-1,3,2-dioxaborolan-2-yl)-[1,1′-biphenyl]-3-yl)-4-oxo-4H-pyrido[1,2-a]pyrimidine-3-carbaldehyde (1.3 g, 31% yield) as a yellow solid. ¹H NMR (400 MHz, DMSO-d₆) δ ppm 10.20 (s, 1H) 9.24 (d, J=7.2 Hz, 1H), 8.79 (s, 1H), 7.93 (s, 1H), 7.73-7.70 (m, 1H), 7.70-7.65 (m, 1H), 7.50-7.41 (m, 4H), 7.35-7.27 (m, 1H), 2.02 (s, 3H), 1.32 (s, 12H).

### (S)-tert-Butyl ((5-(2-chloro-3′-(3-formyl-4-oxo-4H-pyrido[1,2-a]pyrimidin-8-yl)-2′-methyl-[1,1′-biphenyl]-3-yl)-3-methoxypyrazin-2-yl)methyl)((5-oxopyrrolidin-2-yl) methyl)carbamate

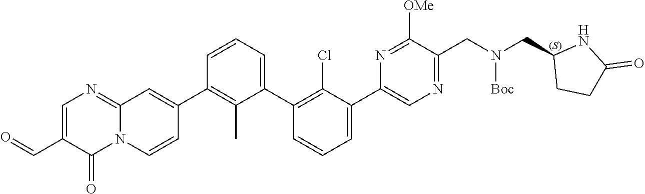

To a solution of (S)-tert-butyl ((5-bromo-3-methoxypyrazin-2-yl)methyl)((5-oxopyrrolidin-2-yl)methyl)carbamate (350 mg, 0.8 mmol) and 8-(2′-chloro-2-methyl-3′-(4,4,5,5-tetramethyl-1,3,2-dioxaborolan-2-yl)-[1,1′-biphenyl]-3-yl)-4-oxo-4H-pyrido[1,2-a]pyrimidine-3-carbaldehyde (0.4 g, 0.8 mmol) in dioxane (20 mL) and H₂O (6 mL) under N₂ was added 1,1′-bis(diphenylphosphino)ferrocene-palladium(II)dichloride (0.1 g, 0.1 mmol) and potassium carbonate (0.3 g, 2.4 mmol). The reaction mixture was stirred at 90° C. for 1.5 hrs. The mixture was diluted with THE (50 mL), washed with saturated aqueous brine solution (2×15 mL), dried over anhydrous sulfate, filtered and concentrated under reduced pressure. The residue was purified by normal phase flash SiO₂ chromatography (0˜90% THF/petroleum ether) to afford (S)-tert-butyl ((5-(2-chloro-3′-(3-formyl-4-oxo-4H-pyrido[1,2-a]pyrimidin-8-yl)-2′-methyl-[1,1′-biphenyl]-3-yl)-3-methoxypyrazin-2-yl)methyl)((5-oxopyrrolidin-2-yl)methyl)carbamate (0.44 g, 39% yield) as a yellow solid. m/z found: 709 [M+H]⁺. ¹H NMR (400 MHz, DMSO-d₆) δ ppm 10.19 (s, 1H), 9.25 (d, J=7.2 Hz, 1H), 8.79 (s, 1H), 8.44 (d, J=7.6 Hz, 1H), 7.93 (d, J=1.6 Hz, 1H), 7.76-7.66 (m, 3H), 7.64-7.55 (m, 1H), 7.55-7.47 (m, 2H), 7.42-7.36 (m, 1H), 4.62-4.52 (m, 2H), 3.98 (s, 3H), 3.87-3.70 (m, 1H), 3.52-3.39 (m, 1H), 2.99-2.88 (m, 2H), 2.16-1.97 (m, 9H), 1.72-1.62 (m, 1H), 1.40 (s, 3H), 1.37 (s, 9H).

### tert-Butyl ((5-(2-chloro-2′-methyl-3′-(4-oxo-3-(((((S)-5-oxopyrrolidin-2-yl)methyl) amino)methyl)-4H-pyrido[1,2-a]pyrimidin-8-yl)-[1,1′-biphenyl]-3-yl)-3-methoxypyrazin-2-yl)methyl)(((S)-5-oxopyrrolidin-2-yl)methyl)carbamate

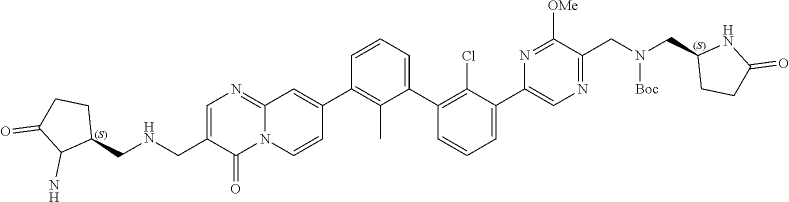

To a mixture of (S)-tert-butyl ((5-(2-chloro-3′-(3-formyl-4-oxo-4H-pyrido[1,2-a] pyrimidin-8-yl)-2′-methyl-[1,1′-biphenyl]-3-yl)-3-methoxypyrazin-2-yl)methyl)((5-oxopyrrolidin-2-yl)methyl)carbamate (240 mg, 0.3 mmol) and (S)-5-(aminomethyl)pyrrolidin-2-one HCl salt (0.2 g, 1.3 mmol) in dichloromethane (30 mL) was added TEA (0.4 mL, 2.5 mmol) and 4 Å molecular sieves (0.5 g). The mixture was stirred at r.t. for 15 hrs. Sodium cyanoborohydride (0.15 g, 2.4 mmol) was added and the mixture was stirred at r.t. for 5 hrs. The reaction mixture was filtered through CELITE® and the filtrate was washed with saturated aqueous brine solution (3×10 mL). The organic phase was concentrated and the residue was purified by normal phase flash SiO₂ chromatography (0˜ 85% 25% of methanol in THF/petroleum ether) to afford tert-butyl ((5-(2-chloro-2′-methyl-3′-(4-oxo-3-(((((S)-5-oxopyrrolidin-2-yl)methyl)amino) methyl)-4H-pyrido[1,2-a]pyrimidin-8-yl)-[1,1′-biphenyl]-3-yl)-3-methoxypyrazin-2-yl)methyl)(((S)-5-oxopyrrolidin-2-yl)methyl)carbamate (180 mg, 43% yield) as a yellow solid. m/z found: 807 [M+H]⁺.

### 8-(2′-Chloro-3′-(6-methoxy-5-(((((S)-5-oxopyrrolidin-2-yl)methyl)amino)methyl)pyrazin-2-yl)-2-methyl-[1,1′-biphenyl]-3-yl)-3-(((((S)-5-oxopyrrolidin-2-yl)methyl)amino)methyl)-4H-pyrido[1,2-a]pyrimidin-4-one as a hydrochloride salt

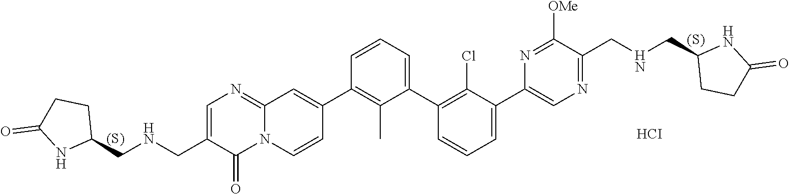

To a solution of tert-butyl ((5-(2-chloro-2′-methyl-3′-(4-oxo-3-(((((S)-5-oxopyrrolidin-2-yl)methyl)amino)methyl)-4H-pyrido[1,2-a]pyrimidin-8-yl)-[1,1′-biphenyl]-3-yl)-3-methoxypyrazin-2-yl)methyl)(((S)-5-oxopyrrolidin-2-yl)methyl)carbamate (0.17 g, 0.2 mmol) in THE (5 mL) was added HCl (4 M solution in EtOAc, 2 mL, 8 mmol) at 0° C. The mixture was stirred at r.t. for 10 hrs. The reaction was concentrated. The residue was purified by reverse phase HPLC to afford 8-(2′-chloro-3′-(6-methoxy-5-(((((S)-5-oxopyrrolidin-2-yl)methyl)amino)methyl) pyrazin-2-yl)-2-methyl-[1,1′-biphenyl]-3-yl)-3-(((((S)-5-oxopyrrolidin-2-yl)methyl) amino)methyl)-4H-pyrido[1,2-a]pyrimidin-4-one as HCl salt (0.07 g, 42%) as a yellow solid. m/z found: 707 [M+H]⁺. ¹H NMR (400 MHz, Methanol-d₄): δ 9.40 (d, J=7.2 Hz, 1H), 8.80 (s, 1H), 8.55 (s, 1H), 8.07 (d, J=1.2 Hz, 1H), 8.01 (dd, J=7.2, 1.6 Hz, 1H), 7.73 (dd, J=8.0, 1.6 Hz, 1H), 7.64-7.58 (m, 1H), 7.58-7.53 (m, 2H), 7.49 (dd, J=7.6, 1.6 Hz, 1H), 7.47-7.43 (m, 1H), 4.60-4.50 (m, 2H), 4.49-4.40 (m, 2H), 4.21-4.08 (m, 5H), 3.45-3.33 (m, 4H), 2.52-2.31 (m, 6H), 2.20 (s, 3H), 2.06-1.91 (m, 2H).

### Example 37: 8,8′-(2,2′-Dimethyl-[1,1′-biphenyl]-3,3′-diyl)bis(3-(((((S)-5-oxopyrrolidin-2-yl)methyl)amino)methyl)-4H-pyrido[1,2-a]pyrimidin-4-one)

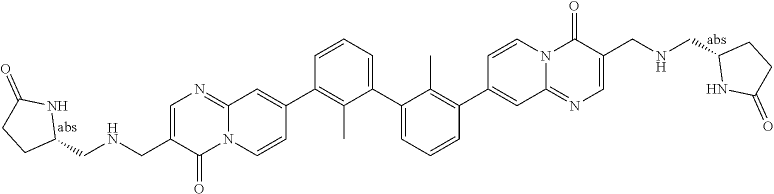

### 8-[3-[3-(3-Formyl-4-oxo-pyrido[1,2-a] pyrimidin-8-yl)-2-methyl-phenyl]-2-methyl-phenyl]-4-oxo-pyrido[1,2-a]pyrimidine-3-carbaldehyde

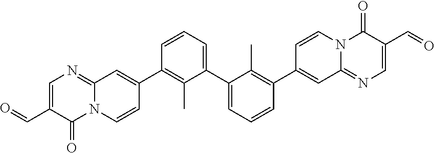 [SMILES: Cc1c(-c2ccn3c(=O)c(C=O)cnc3c2)cccc1-c1cccc(-c2ccn3c(=O)c(C=O)cnc3c2)c1C]

To a solution of 8-[2-methyl-3-(4,4,5,5-tetramethyl-1,3,2-dioxaborolan-2-yl)phenyl]-4-oxo-pyrido[1,2-a]pyrimidine-3-carbaldehyde (50 mg, 0.13 mmol), 8-(3-bromo-2-methyl-phenyl)-4-oxo-pyrido[1,2-a]pyrimidine-3-carbaldehyde (44 mg, 0.13 mmol) and potassium carbonate (53 mg, 0.38 mmol) in degassed dioxane/water (5:1 (v/v)) was added Pd(PPh₃)₄ (14.81 mg, 0.01 mmol) and the mixture was stirred at 95° C. for 2 hours under N₂(g). Water was added and the mixture was extracted into EtOAc three times. The combined organic layers were dried over anhydrous sodium sulfate, filtered, concentrated, and purified on silica gel column using MeOH in DCM (0 to 10% gradient) as eluent to give 8-[3-[3-(3-formyl-4-oxo-pyrido[1,2-a]pyrimidin-8-yl)-2-methyl-phenyl]-2-methyl-phenyl]-4-oxo-pyrido[1,2-a]pyrimidine-3-carbaldehyde (30.00 mg, 44.5%). MS: m/z found 572.2 [M+H]⁺; ¹H NMR (400 MHz, Chloroform-d): δ 10.41 (d, J=1.4 Hz, 2H), 9.33 (ddd, J=7.2, 1.5, 0.7 Hz, 2H), 8.95 (d, J=1.4 Hz, 2H), 7.89-7.80 (m, 2H), 7.50-7.42 (m, 4H), 7.35 (ddd, J=12.4, 7.5, 1.5 Hz, 4H), 2.10 (s, 6H).

### 8-[2-Methyl-3-[2-methyl-3-[4-oxo-3-[[[(2S)-5-oxopyrrolidin-2-yl]methylamino]methyl]pyrido[1,2-a]pyrimidin-8-yl]phenyl]phenyl]-3-[[[(2S)-5-oxopyrrolidin-2-yl]methylamino]methyl]pyrido[1,2-a]pyrimidin-4-one

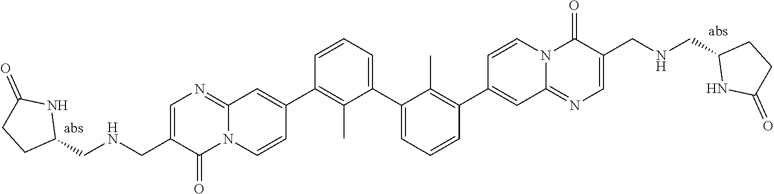 [SMILES: Cc1c(-c2ccn3c(=O)c(CNC[C@@H]4CCC(=O)N4)cnc3c2)cccc1-c1cccc(-c2ccn3c(=O)c(CNC[C@@H]4CCC(=O)N4)cnc3c2)c1C]

A mixture of 8-[3-[3-(3-formyl-4-oxo-pyrido[1,2-a]pyrimidin-8-yl)-2-methyl-phenyl]-2-methyl-phenyl]-4-oxo-pyrido[1,2-a]pyrimidine-3-carbaldehyde (90.00 mg, 0.17 mmol), (5S)-5-(aminomethyl)yrrolidine-2-one (78.04 mg, 0.68 mmol) and acetic acid (19.55 uL, 20.53 mg, 0.34 mmol) were stirred in MeOH/THF (2 mL, 1:1) for 2 hours. NaBH₃CN (42.96 mg, 0.68 mmol) was added, the mixture was stirred for additional 5 minutes and quenched with water (0.2 mL). After extraction into EtOAc (x3), the combined organic layers were dried over anhydrous sodium sulfate, concentrated, and purified by HPLC to provide 8-[2-methyl-3-[2-methyl-3-[4-oxo-3-[[[(2S)-5-oxopyrrolidin-2-yl]methylamino]methyl]pyrido[1,2-a]pyrimidin-8-yl]phenyl]phenyl]-3-[[[(2S)-5-oxopyrrolidin-2-yl]methylamino]methyl]pyrido[1,2-a]pyrimidin-4-one (85.00 mg, 68.8%). MS: m/z found 723.7 [M+H]⁺; ¹H NMR (400 MHz, Methanol-d₄): δ 9.27 (dt, J=7.4, 1.2 Hz, 2H), 8.62 (s, 2H), 7.88 (dd, J=1.8, 0.7 Hz, 2H), 7.74 (dd, J=7.3, 1.9 Hz, 2H), 7.54-7.46 (m, 4H), 7.40-7.36 (m, 2H), 4.39 (d, J=2.9 Hz, 4H), 4.11 (tt, J=7.3, 5.2 Hz, 2H), 3.29-3.24 (m, 2H), 2.48-2.37 (m, 6H), 2.37-2.32 (m, 2H), 2.13 (s, 6H), 1.93 (dddd, J=11.6, 9.6, 4.7, 1.6 Hz, 2H).

### Example 38: N,N′-((((2,2′-Dichloro-[1,1′-biphenyl]-3,3′-diyl)bis(3-methoxypyrazine-5,2-diyl))bis(azanediyl))bis(ethane-2,1-diyl))diacetamide

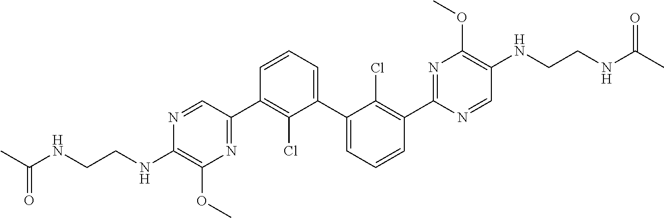 [SMILES: COc1nc(-c2cccc(-c3cccc(-c4cnc(NCCNC(C)=O)c(OC)n4)c3Cl)c2Cl)ncc1NCCNC(C)=O]

### N-(2-((5-Bromo-3-methoxypyrazin-2-yl)-amino)-ethyl)-acetamide and N-(2-((5-iodo-6-methoxypyrazin-2-yl)-amino)-ethyl)-acetamide

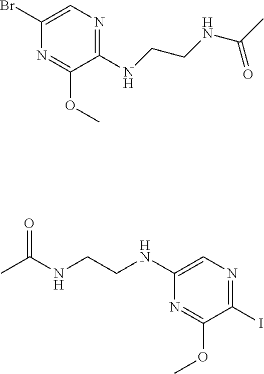

5-Bromo-2-iodo-3-methoxy-pyrazine (75.00 mg, 0.24 mmol) and N-(2-aminoethyl)-acetamide (72.98 mg, 0.71 mmol) were heated neat at 100° C. for 30 minutes. The reaction was cooled to rt and the crude residue was purified by reverse phase HPLC to afford the following products:

N-[2-[(5-iodo-6-methoxy-pyrazin-2-yl)-amino]-ethyl]-acetamide (18.00 mg, 22.5% yield) as a clear oil (early eluting peak). MS: m/z found 289, 291 [M+H]⁺.

N-[2-[(5-bromo-3-methoxy-pyrazin-2-yl)-amino]-ethyl]-acetamide (16.00 mg, 23.2% yield) as a clear oil (late eluting peak). MS: m/z found 337 [M+H]⁺.

### N,N′-((((2,2′-Dichloro-[1,1′-biphenyl]-3,3′-diyl)bis(3-methoxypyrazine-5,2-diyl))bis(azanediyl))bis(ethane-2,1-diyl))diacetamide

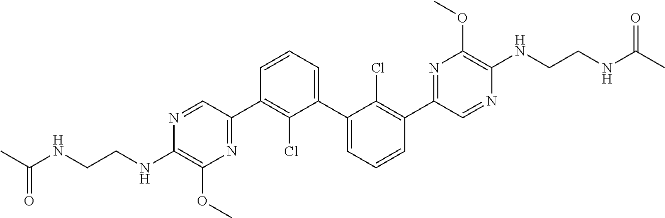 [SMILES: COc1nc(-c2cccc(-c3cccc(-c4cnc(NCCNC(C)=O)c(OC)n4)c3Cl)c2Cl)cnc1NCCNC(C)=O]

Tetrakis(triphenylphosphine)palladium(0) (5 mg, 0.004 mmol), potassium carbonate (11.62 mg, 0.08 mmol), 2-[2-chloro-3-[2-chloro-3-(4,4,5,5-tetramethyl-1,3,2-dioxaborolan-2-yl)phenyl]phenyl]-4,4,5,5-tetramethyl-1,3,2-dioxaborolane (10 mg, 0.02 mmol) and N-[2-[(5-bromo-3-methoxy-pyrazin-2-yl)-amino]-ethyl]-acetamide (15 mg, 0.05 mmol) were suspended in 1 ml of 4:1 dioxane/water solvent mixture, and the solution was heated at 110° C. for 15 minutes. The reaction was cooled to rt, diluted with water (3 ml) and extracted with EtOAc (3×2 ml). The combined organics were further washed with water, brine, then dried over sodium sulfate, filtered, and concentrated. The residue was purified by reverse phase HPLC to afford N-[2-[[5-[3-[3-[5-(2-acetamidoethylamino)-6-methoxy-pyrazin-2-yl]-2-chloro-phenyl]-2-chloro-phenyl]-3-methoxy-pyrazin-2-yl]-amino]ethyl]acetamide (6.40 mg, 47.5% yield) as a clear oil. MS: m/z found 639.3, 641.3 [M+H]⁺. ¹H NMR (400 MHz, Chloroform-d) δ 8.00 (s, 2H), 7.63 (d, J=7.7 Hz, 2H), 7.41 (t, J=7.6 Hz, 2H), 7.33-7.23 (m, 3H), 6.51 (s, 2H), 5.58 (t, J=5.8 Hz, 2H), 4.04 (s, 6H), 3.67 (q, J=5.8 Hz, 4H), 3.53 (q, J=5.6 Hz, 4H), 1.97 (s, 6H).

### Example 39: N,N′-((((2,2′-Dichloro-[1,1′-biphenyl]-3,3′-diyl)bis(6-methoxypyrazine-5,2-diyl))bis(azanediyl))bis(ethane-2,1-diyl))diacetamide

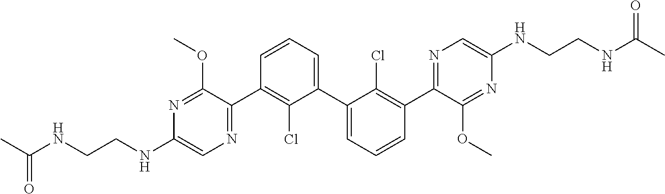 [SMILES: COc1nc(NCCNC(C)=O)cnc1-c1cccc(-c2cccc(-c3ncc(NCCNC(C)=O)nc3OC)c2Cl)c1Cl]

Tetrakis(triphenylphosphine)palladium(0) (6 mg, 0.01 mmol), potassium carbonate (14 mg, 0.10 mmol), 2-[2-chloro-3-[2-chloro-3-(4,4,5,5-tetramethyl-1,3,2-dioxaborolan-2-yl)phenyl]phenyl]-4,4,5,5-tetramethyl-1,3,2-dioxaborolane (12.00 mg, 0.03 mmol) and N-[2-[(5-iodo-6-methoxy-pyrazin-2-yl)amino]ethyl]acetamide (21 mg, 0.06 mmol) were suspended in 1 ml of 4:1 dioxane/water solvent mixture, and the solution was heated at 110° C. for 15 minutes. The reaction was cooled to rt, diluted with water (3 ml) and extracted with EtOAc (3×2 ml). The combined organics were further washed with water, brine, then dried over sodium sulfate, filtered, and concentrated. Crude product was purified by reverse phase HPLC to afford N-[2-[[5-[3-[3-[5-(2-acetamidoethylamino)-3-methoxy-pyrazin-2-yl]-2-chloro-phenyl]-2-chloro-phenyl]-6-methoxy-pyrazin-2-yl]amino]ethyl]acetamide (14.20 mg, 87.9% yield) as a white foam. MS: m/z found 639.3, 641.3 [M+H]⁺. ¹H NMR (400 MHz, Chloroform-d) δ 7.52 (d, J=1.3 Hz, 2H), 7.43 (dt, J=7.6, 1.7 Hz, 2H), 7.38 (td, J=7.5, 1.3 Hz, 2H), 7.32 (dt, J=7.5, 1.7 Hz, 2H), 6.04 (s, 2H), 5.05 (d, J=5.7 Hz, 2H), 3.89 (d, J=1.3 Hz, 6H), 3.62-3.41 (m, 8H), 1.99 (d, J=1.3 Hz, 6H).

### Example 40: 8-(3′-(2-Amino-2,3-dihydro-1H-inden-5-yl)-2′-chloro-2-methyl-[1,1′-biphenyl]-3-yl)-3-(((((S)-5-oxopyrrolidin-2-yl)methyl)amino)methyl)-4H-pyrido[1,2-a]pyrimidin-4-one

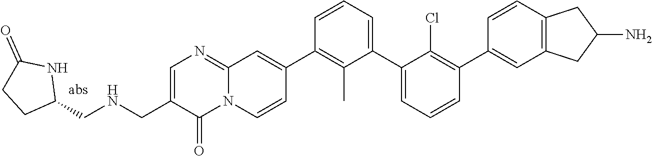 [SMILES: Cc1c(-c2ccn3c(=O)c(CNC[C@@H]4CCC(=O)N4)cnc3c2)cccc1-c1cccc(-c2ccc3c(c2)CC(N)C3)c1Cl]

### tert-Butyl N-[5-(4,4,5,5-tetramethyl-1,3,2-dioxaborolan-2-yl)indan-2-yl]carbamate

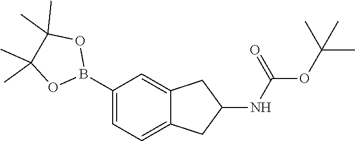 [SMILES: CC(C)(C)OC(=O)NC1Cc2ccc(B3OC(C)(C)C(C)(C)O3)cc2C1]

To a solution of tert-butyl N-(5-bromoindan-2-yl)carbamate (1.40 g, 4.48 mmol) in DMSO (15 mL) was added 4,4,5,5-tetramethyl-2-(4,4,5,5-tetramethyl-1,3,2-dioxaborolan-2-yl)-1,3,2-dioxaborolane (3.42 g, 13.45 mmol) and potassium acetate (1.32 g, 13.45 mmol). After degassing with N₂ for 10 minutes, Pd(dppf)Cl₂ (0.33 g, 0.45 mmol) was added under a N₂ atmosphere. The reaction mixture was stirred at 95° C. for 2 hours, cooled to room temperature, diluted with water, and extracted with ethyl acetate. The organic layer was washed with brine, dried over sodium sulfate, and concentrated on a rotary evaporator. The crude residue was purified by flash column chromatography (ethyl acetate in hexanes) to give tert-butyl N-[5-(4,4,5,5-tetramethyl-1,3,2-dioxaborolan-2-yl)indan-2-yl]carbamate (1.52 g, 94.3%)¹H NMR (400 MHz, Chloroform-d): δ7.66 (s, 1H), 7.61 (dd, J=7.5, 1.1 Hz, 1H), 7.21 (d, J=7.5 Hz, 1H), 4.82 (d, J=8.0 Hz, 1H), 4.44 (s, 1H), 3.25 (dt, J=16.1, 6.4 Hz, 2H), 2.77 (dt, J=16.1, 5.1 Hz, 2H), 1.41 (s, 9H), 1.32 (s, 12H).

### tert-Butyl N-[5-(3-bromo-2-chloro-phenyl)indan-2-yl]carbamate

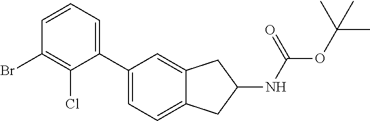 [SMILES: CC(C)(C)OC(=O)NC1Cc2ccc(-c3cccc(Br)c3Cl)cc2C1]

To a solution of tert-butyl N-[5-(4,4,5,5-tetramethyl-1,3,2-dioxaborolan-2-yl)indan-2-yl]carbamate (0.5 g, 1.39 mmol) and 1,3-dibromo-2-chloro-benzene (0.38 g, 1.39 mmol) in degassed dioxane (5 mL) were added a solution of potassium carbonate (0.58 g, 4.18 mmol) in degassed H₂O (1 mL) and Pd(PPh₃)₄ (0.16 g, 0.14 mmol), and the mixture was stirred at 95° C. for 2 hours under N₂(g). After cooling down to rt and workup, the residue was purified by flash column chromatography using ethyl acetate in hexanes as eluent (0 to 20% gradient) to give tert-butyl N-[5-(3-bromo-2-chloro-phenyl)indan-2-yl]carbamate (0.43 g, 73%). ¹H NMR (400 MHz, Chloroform-d): δ 7.61 (dt, J=7.9, 1.6 Hz, 1H), 7.30-7.11 (m, 5H), 4.86 (s, 1H), 4.52 (s, 1H), 3.33 (dd, J=16.2, 7.1 Hz, 2H), 2.84 (dt, J=16.1, 3.9 Hz, 2H), 1.46 (s, 9H).

### 8-[2-Methyl-3-(4,4,5,5-tetramethyl-1,3,2-dioxaborolan-2-yl)phenyl]-3-[[[(2S)-5-oxopyrrolidin-2-yl]methylamino]methyl]pyrido[1,2-a]pyrimidin-4-one

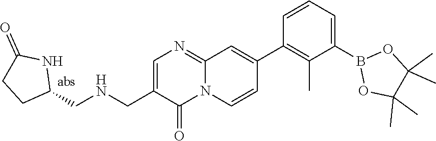 [SMILES: Cc1c(B2OC(C)(C)C(C)(C)O2)cccc1-c1ccn2c(=O)c(CNC[C@@H]3CCC(=O)N3)cnc2c1]

A mixture of 8-[2-methyl-3-(4,4,5,5-tetramethyl-1,3,2-dioxaborolan-2-yl)phenyl]-4-oxo-pyrido[1,2-a]pyrimidine-3-carbaldehyde (0.50 g, 1.28 mmol), (5S)-5-(aminomethyl)pyrrolidin-2-one (0.29 g, 2.56 mmol) and acetic acid (73 uL, 76.94 mg, 1.28 mmol) were stirred in MeOH/THF (5 mL, 1:1) for 2 hours. NaBH₃CN (0.16 g, 2.56 mmol) was added, the mixture was stirred for 10 min and quenched with water (0.5 mL). After extraction into EtOAc (x3), the combined organic layers were dried over anhydrous sodium sulfate, filtered, concentrated, and purified on silica gel column (10% MeOH in DCM) to provide 8-[2-methyl-3-(4,4,5,5-tetramethyl-1,3,2-dioxaborolan-2-yl)phenyl]-3-[[[(2S)-5-oxopyrrolidin-2-yl]methylamino]methyl]pyrido[1,2-a]pyrimidin-4-one (0.58 g, 92.7%). MS: m/z found 489.4 [M+H]⁺; ¹H NMR (400 MHz, Methanol-d₄): δ 9.14 (d, J=7.4 Hz, 1H), 8.51 (d, J=1.7 Hz, 1H), 7.84 (dt, J=7.3, 1.3 Hz, 1H), 7.65 (dt, J=1.9, 0.9 Hz, 1H), 7.46 (ddd, J=7.3, 2.0, 0.8 Hz, 1H), 7.41 (dt, J=7.6, 1.4 Hz, 1H), 7.32 (t, J=7.5 Hz, 1H), 4.39-4.21 (m, 2H), 4.13-4.00 (m, 1H), 3.21 (tddd, J=12.4, 9.9, 5.1, 2.5 Hz, 2H), 2.51 (s, 3H), 2.47-2.33 (m, 3H), 1.93 (dddd, J=11.2, 7.8, 3.8, 1.7 Hz, 1H), 1.37 (s, 12H).

### tert-Butyl N-[[8-[2-methyl-3-(4,4,5,5-tetramethyl-1,3,2-dioxaborolan-2-yl)phenyl]-4-oxo-pyrido[1,2-a]pyrimidin-3-yl]methyl]-N-[[(2S)-5-oxopyrrolidin-2-yl]methyl]carbamate

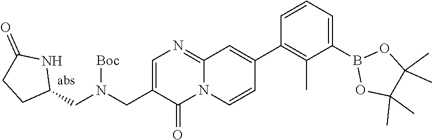 [SMILES: Cc1c(B2OC(C)(C)C(C)(C)O2)cccc1-c1ccn2c(=O)c(CN(C[C@@H]3CCC(=O)N3)C(=O)OC(C)(C)C)cnc2c1]

A mixture of 8-[2-methyl-3-(4,4,5,5-tetramethyl-1,3,2-dioxaborolan-2-yl)phenyl]-3-[[[(2S)-5-oxopyrrolidin-2-yl]methylamino]methyl]pyrido[1,2-a]pyrimidin-4-one (0.40 g, 0.82 mmol) and di-tert-butyl dicarbonate (0.23 g, 1.06 mmol) were stirred in DCM at rt for overnight. The mixture was concentrated and purified by flash column chromatography to give tert-butyl N-[[8-[2-methyl-3-(4,4,5,5-tetramethyl-1,3,2-dioxaborolan-2-yl)phenyl]-4-oxo-pyrido[1,2-a]pyrimidin-3-yl]methyl]-N-[[(2S)-5-oxopyrrolidin-2-yl]methyl]carbamate (300.00 mg, 62.2%). MS: m/z found 589.4 [M+H]⁺.

### tert-Butyl N-[[8-[3-[3-[2-(tert-butoxycarbonylamino)indan-5-yl]-2-chloro-phenyl]-2-methyl-phenyl]-4-oxo-pyrido[1,2-a]pyrimidin-3-yl]methyl]-N-[[(2S)-5-oxopyrrolidin-2-yl]methyl]carbamate

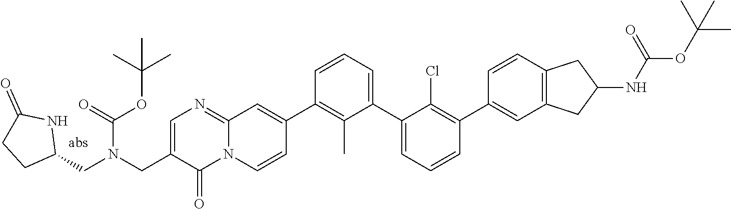 [SMILES: Cc1c(-c2ccn3c(=O)c(CN(C[C@@H]4CCC(=O)N4)C(=O)OC(C)(C)C)cnc3c2)cccc1-c1cccc(-c2ccc3c(c2)CC(NC(=O)OC(C)(C)C)C3)c1Cl]

To a solution of tert-butyl N-[[8-[2-methyl-3-(4,4,5,5-tetramethyl-1,3,2-dioxaborolan-2-yl)phenyl]-4-oxo-pyrido[1,2-a]pyrimidin-3-yl]methyl]-N-[[(2S)-5-oxopyrrolidin-2-yl]methyl]carbamate (50 mg, 0.08 mmol), tert-butyl N-[5-(3-bromo-2-chloro-phenyl)indan-2-yl]carbamate (47 mg, 0.11 mmol) and potassium carbonate (35 mg, 0.25 mmol) in degassed dioxane/water (5:1) was added Pd(PPh₃)₄ (10 mg, 0.01 mmol) and the mixture was stirred at 95° C. for 2 hours under N₂(g). Water was added and the mixture was extracted into EtOAc three times. The combined organic layers were dried over anhydrous sodium sulfate, concentrated, and purified on silica gel column using MeOH in DCM (0 to 10% gradient) as eluent to give tert-butyl N-[[8-[3-[3-[2-(tert-butoxycarbonylamino)indan-5-yl]-2-chloro-phenyl]-2-methyl-phenyl]-4-oxo-pyrido[1,2-a]pyrimidin-3-yl]methyl]-N-[[(2S)-5-oxopyrrolidin-2-yl]methyl]carbamate (35 mg, 51.2%). MS: m/z found 804.5 [M+H]⁺.

### 8-[3-[3-(2-Aminoindan-5-yl)-2-chloro-phenyl]-2-methyl-phenyl]-3-[[[(2S)-5-oxopyrrolidin-2-yl]methylamino]methyl]pyrido[1,2-a]pyrimidin-4-one

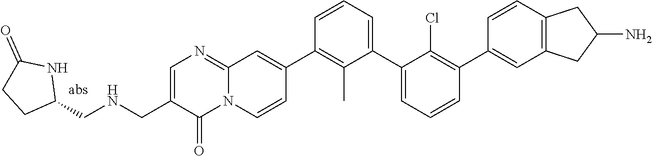 [SMILES: Cc1c(-c2ccn3c(=O)c(CNC[C@@H]4CCC(=O)N4)cnc3c2)cccc1-c1cccc(-c2ccc3c(c2)CC(N)C3)c1Cl]

tert-Butyl N-[[8-[3-[3-[2-(tert-butoxycarbonylamino)indan-5-yl]-2-chloro-phenyl]-2-methyl-phenyl]-4-oxo-pyrido[1,2-a]pyrimidin-3-yl]methyl]-N-[[(2S)-5-oxopyrrolidin-2-yl]methyl]carbamate (35 mg, 0.04 mmol) in DCM/TFA (2:1) mixture was stirred for 30 min, concentrated, and purified by HPLC to give 8-[3-[3-(2-aminoindan-5-yl)-2-chloro-phenyl]-2-methyl-phenyl]-3-[[[(2S)-5-oxopyrrolidin-2-yl]methylamino]methyl]pyrido[1,2-a]pyrimidin-4-one (20 mg, 76%). MS: m/z found 604.4 [M+H]⁺; ¹H NMR (400 MHz, Methanol-d₄): δ 9.40 (d, J=7.0 Hz, 1H), 8.81 (s, 1H), 8.07 (d, J=1.6 Hz, 1H), 8.00 (dd, J=7.2, 1.7 Hz, 1H), 7.56-7.52 (m, 2H), 7.52-7.47 (m, 1H), 7.42 (d, J=1.9 Hz, 1H), 7.41-7.37 (m, 3H), 7.35-7.31 (m, 2H), 4.45 (d, J=2.0 Hz, 2H), 4.16 (dtd, J=12.3, 7.4, 4.9 Hz, 2H), 3.53-3.49 (m, 1H), 3.49-3.44 (m, 1H), 3.42-3.35 (m, 2H), 3.16-3.11 (m, 1H), 3.09 (t, J=3.6 Hz, 1H), 2.50-2.34 (m, 3H), 2.19 (s, 3H), 2.04-1.94 (m, 1H).

### Example 41: 8-(3′-(2-Amino-2,3-dihydro-1H-inden-5-yl)-2,2′-dimethyl-[1,1′-biphenyl]-3-yl)-3-(((((S)-5-oxopyrrolidin-2-yl)methyl)amino)methyl)-4H-pyrido[1,2-a]pyrimidin-4-one

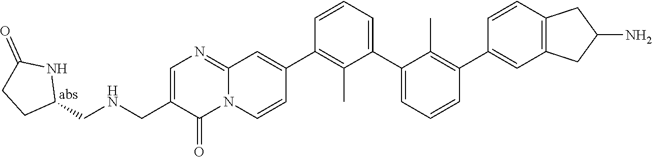 [SMILES: Cc1c(-c2ccc3c(c2)CC(N)C3)cccc1-c1cccc(-c2ccn3c(=O)c(CNC[C@@H]4CCC(=O)N4)cnc3c2)c1C]

### tert-Butyl N-[5-(3-bromo-2-methyl-phenyl)indan-2-yl]carbamate

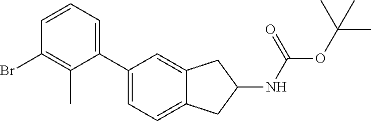 [SMILES: Cc1c(Br)cccc1-c1ccc2c(c1)CC(NC(=O)OC(C)(C)C)C2]

To a solution of tert-butyl N-[5-(4,4,5,5-tetramethyl-1,3,2-dioxaborolan-2-yl)indan-2-yl]carbamate (0.50 g, 1.39 mmol) and 1,3-dibromo-2-methyl-benzene (0.35 g, 1.39 mmol) in degassed dioxane (5 mL) were added a solution of potassium carbonate (0.58 g, 4.18 mmol) in degassed H₂O (1 mL) and Pd(PPh₃)₄ (0.16 g, 0.14 mmol) and the mixture was stirred at 95° C. for 2 hours under N₂(g). After cooling to rt and workup, the residue was purified by flash column chromatography using ethyl acetate in hexanes as eluent (0 to 20% gradient). ¹H NMR (400 MHz, Chloroform-d): δ 7.54 (dd, J=8.0, 1.4 Hz, 1H), 7.28-7.23 (m, 1H), 7.16-7.10 (m, 2H), 7.10-7.04 (m, 2H), 4.85 (s, 1H), 4.53 (s, 1H), 3.41-3.26 (m, 2H), 2.84 (dt, J=16.1, 4.9 Hz, 2H), 2.32 (s, 3H), 1.47 (s, 9H).

### tert-Butyl N-[[8-[3-[3-[2-(tert-butoxycarbonylamino)indan-5-yl]-2-methyl-phenyl]-2-methyl-phenyl]-4-oxo-pyrido[1,2-a]pyrimidin-3-yl]methyl]-N-[[(2S)-5-oxopyrrolidin-2-yl]methyl]carbamate

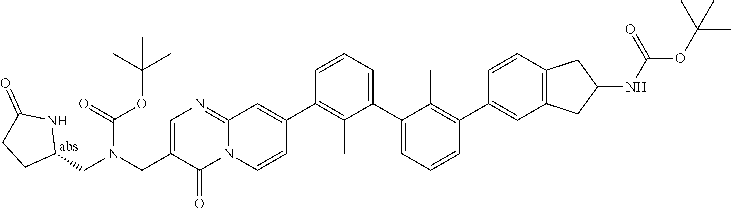 [SMILES: Cc1c(-c2ccc3c(c2)CC(NC(=O)OC(C)(C)C)C3)cccc1-c1cccc(-c2ccn3c(=O)c(CN(C[C@@H]4CCC(=O)N4)C(=O)OC(C)(C)C)cnc3c2)c1C]

To a solution of tert-butyl N-[[8-[2-methyl-3-(4,4,5,5-tetramethyl-1,3,2-dioxaborolan-2-yl)phenyl]-4-oxo-pyrido[1,2-a]pyrimidin-3-yl]methyl]-N-[[(2S)-5-oxopyrrolidin-2-yl]methyl]carbamate (50 mg, 0.08 mmol), tert-butyl N-[5-(3-bromo-2-methyl-phenyl)indan-2-yl]carbamate (44 mg, 0.11 mmol) and potassium carbonate (35 mg, 0.25 mmol) in degassed dioxane/water (5:1) was added Pd(PPh₃)₄ (10 mg, 0.01 mmol) and the mixture was stirred at 95° C. for 2 hours under N₂(g). Water was added and the mixture was extracted into EtOAc three times. The combined organic layers were dried over anhydrous sodium sulfate, concentrated, and purified on silica gel column using MeOH in DCM (0 to 10% gradient) as eluent to give tert-butyl N-[[8-[3-[3-[2-(tert-butoxycarbonylamino)indan-5-yl]-2-methyl-phenyl]-2-methyl-phenyl]-4-oxo-pyrido[1,2-a]pyrimidin-3-yl]methyl]-N-[[(2S)-5-oxopyrrolidin-2-yl]methyl]carbamate (28.00 mg, 42.0%). MS: m/z found 784.5 [M+H]⁺.

### 8-[3-[3-(2-Aminoindan-5-yl)-2-methyl-phenyl]-2-methyl-phenyl]-3-[[[(2S)-5-oxopyrrolidin-2-yl]methylamino]methyl]pyrido[1,2-a]pyrimidin-4-one

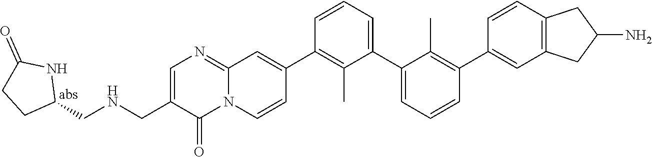 [SMILES: Cc1c(-c2ccc3c(c2)CC(N)C3)cccc1-c1cccc(-c2ccn3c(=O)c(CNC[C@@H]4CCC(=O)N4)cnc3c2)c1C]

tert-Butyl N-[[8-[3-[3-[2-(tert-butoxycarbonylamino)indan-5-yl]-2-methyl-phenyl]-2-methyl-phenyl]-4-oxo-pyrido[1,2-a]pyrimidin-3-yl]methyl]-N-[[(2S)-5-oxopyrrolidin-2-yl]methyl]carbamate (40 mg, 0.05 mmol) in DCM/TFA (2:1) mixture was stirred for 30 min, concentrated, and purified by HPLC to give 8-[3-[3-(2-aminoindan-5-yl)-2-methyl-phenyl]-2-methyl-phenyl]-3-[[[(2S)-5-oxopyrrolidin-2-yl]methylamino]methyl]pyrido[1,2-a]pyrimidin-4-one (22 mg, 73.9%). MS: m/z found 584.5 [M+H]⁺; ¹H NMR (400 MHz, Methanol-d₄): δ 9.39 (d, J=7.2 Hz, 1H), 8.80 (s, 1H), 8.07 (d, J=1.7 Hz, 1H), 8.02 (dd, J=7.2, 1.7 Hz, 1H), 7.56-7.46 (m, 2H), 7.43-7.36 (m, 2H), 7.33 (t, J=7.6 Hz, 1H), 7.29 (s, 1H), 7.23 (dt, J=7.8, 1.8 Hz, 2H), 7.15 (dd, J=7.5, 1.4 Hz, 1H), 4.45 (d, J=2.1 Hz, 2H), 4.15 (dtd, J=12.6, 7.5, 4.8 Hz, 2H), 3.50 (dd, J=7.3, 1.8 Hz, 1H), 3.48-3.43 (m, 1H), 3.42-3.33 (m, 2H), 3.15-3.11 (m, 1H), 3.10-3.06 (m, 1H), 2.49-2.33 (m, 3H), 2.15 (s, 3H), 2.03-1.98 (m, 1H), 1.96 (s, 3H).

### Example 42: (5S,5'S)-5,5′-((((2,2′-Dichloro-[1,1′-biphenyl]-3,3′-diyl)bis(1-methyl-1,2,3,4-tetrahydro-1,8-naphthyridine-7,4-diyl))bis(azanediyl))bis(methylene))bis(pyrrolidin-2-one)

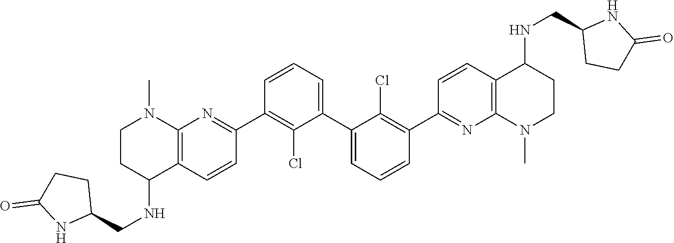 [SMILES: CN1CCC(NC[C@@H]2CCC(=O)N2)c2ccc(-c3cccc(-c4cccc(-c5ccc6c(n5)N(C)CCC6NC[C@@H]5CCC(=O)N5)c4Cl)c3Cl)nc21]

### (5S)-5-(((7-Bromo-1-methyl-1,2,3,4-tetrahydro-1,8-naphthyridin-)-rac-amino)-methyl)-pyrrolidin-2-one

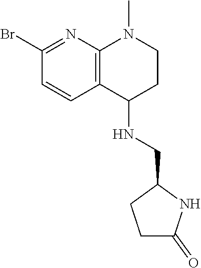 [SMILES: CN1CCC(NC[C@@H]2CCC(=O)N2)c2ccc(Br)nc21]

7-Bromo-1-methyl-2,3-dihydro-1,8-naphthyridin-4-one (0.11 g, 0.46 mmol), (5S)-5-(aminomethyl)pyrrolidin-2-one (0.10 g, 0.91 mmol) and tetraethoxytitanium (0.31 g, 1.37 mmol) were suspended in 3 ml anhydrous THE and the reaction mixture was heated at 105° C. for 5 hours in a sealed tube. The reaction was then cooled to −50° C., and NaBH₄, (52 mg, 1.37 mmol) was added portion-wise under a nitrogen stream. The mixture was gradually warmed to rt over 1 hour, and the solution was slowly added to an ice water solution (20 ml). 10 ml EtOAc was subsequently added with stirring. The resulting slurry was filtered through CELITE® and the layers were partitioned. The organic layer was further washed with brine, dried over sodium sulfate, and concentrated. Crude sample was purified by reverse phase HPLC to afford (5S)-5-[[(7-bromo-1-methyl-3,4-dihydro-2H-1,8-naphthyridin-4-yl)-rac-amino]methyl]pyrrolidin-2-one (54.30 mg, 40.6% yield) as a white foam (racemic compound). MS: m/z found 339, 341 [M+H]⁺.

### tert-Butyl (7-bromo-1-methyl-1,2,3,4-tetrahydro-1,8-naphthyridin-4-yl)-(((S)-5-oxopyrrolidin-2-yl)methyl)carbamate

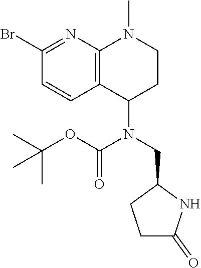 [SMILES: CN1CCC(N(C[C@@H]2CCC(=O)N2)C(=O)OC(C)(C)C)c2ccc(Br)nc21]

(5S)-5-[[(7-Bromo-1-methyl-3,4-dihydro-2H-1,8-naphthyridin-4-yl)amino]methyl]pyrrolidin-2-one (119 mg, 0.35 mmol), DMAP (5 mg), and DIEA (0.24 mL, 0.18 g, 1.40 mmol) were dissolved in 3 ml anhydrous THE and tert-butoxycarbonyl tert-butyl carbonate (0.15 g, 0.70 mmol) was added portion-wise. The resulting mixture was stirred at rt for 12 hours. EtOAc (10 ml) was added to the reaction mixture and the resulting solution was washed with aqueous saturated sodium bicarbonate solution (5 ml×2) and brine (5 ml×2). The organic layer was concentrated in vacuo and the rersidue was purified by silica gel chromatography, eluting with 0-5% MeOH gradient in DCM to afford tert-butyl N-(7-bromo-1-methyl-3,4-dihydro-2H-1,8-naphthyridin-4-yl)-N-[[(2S)-5-oxopyrrolidin-2-yl]methyl]carbamate (0.12 g, 77.9% yield) as a yellow foam (racemic compound). MS: m/z found 439.1, 441.1 [M+H]⁺.

### (5S,5'S)-5,5′-((((2,2′-Dichloro-[1,1′-biphenyl]-3,3′-diyl)bis(1-methyl-1,2,3,4-tetrahydro-1,8-naphthyridine-7,4-diyl))bis(azanediyl))bis(methylene))bis(pyrrolidin-2-one)

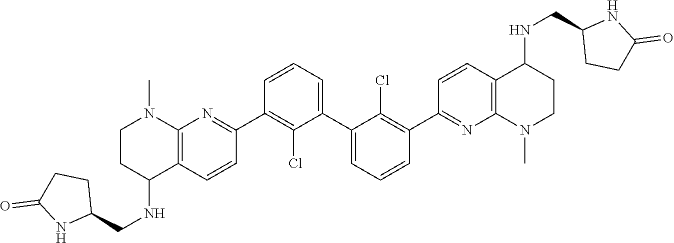 [SMILES: CN1CCC(NC[C@@H]2CCC(=O)N2)c2ccc(-c3cccc(-c4cccc(-c5ccc6c(n5)N(C)CCC6NC[C@@H]5CCC(=O)N5)c4Cl)c3Cl)nc21]

Pd(PPh₃)₄ (11.18 mg, 0.01 mmol), potassium carbonate (26.73 mg, 0.19 mmol),2-[2-chloro-3-[2-chloro-3-(4,4,5,5-tetramethyl-1,3,2-dioxaborolan-2-yl)phenyl]phenyl]-4,4,5,5-tetramethyl-1,3,2-dioxaborolane (23.00 mg, 0.05 mmol) and rac-(5S)-5-[[(7-bromo-1-methyl-3,4-dihydro-2H-1,8-naphthyridin-4-yl)amino]methyl]pyrrolidin-2-one;2,2,2-trifluoroacetic acid (54.87 mg, 0.12 mmol), were suspended in 2 ml of 4:1 dioxane/water solvent mixture, and the solution was heated at 110° C. for 30 minutes. The reaction was cooled to rt, filtered through CELITE®, and the filtrate was concentrated. The crude product was purified by reverse phase HPLC to afford (5S)-5-[[[7-[2-chloro-3-[2-chloro-3-[8-methyl-5-[[(2S)-5-oxopyrrolidin-2-yl]methylamino]-6,7-dihydro-5H-1,8-naphthyridin-2-yl]phenyl]phenyl]-1-methyl-3,4-dihydro-2H-1,8-naphthyridin-4-yl]amino]methyl]pyrrolidin-2-one (13 mg, 36%) as a mixture of diastereoisomers. MS: m/z found 739.4, 741.4 [M+H]⁺. ¹H NMR (400 MHz, Chloroform-d) δ 7.65 (d, J=7.6 Hz, 2H), 7.44-7.33 (m, 4H), 7.33-7.27 (m, 2H), 6.89 (dq, J=7.8, 5.2, 3.8 Hz, 2H), 6.14 (s, 2H), 3.73 (t, J=13.5 Hz, 4H), 3.60-3.42 (m, 2H), 3.32 (d, J=12.8 Hz, 2H), 3.18 (d, J=2.6 Hz, 6H), 2.93 (d, J=11.8 Hz, 2H), 2.61 (t, J=10.4 Hz, 2H), 2.36 (t, J=8.2 Hz, 4H), 2.25 (dq, J=14.5, 7.9, 7.5 Hz, 2H), 1.99 (d, J=42.9 Hz, 4H), 1.78 (d, J=10.3 Hz, 2H).

### Example 43: 2-Amino-N-(5-(2-chloro-2′-methyl-3′-(4-oxo-3-(((((S)-5-oxopyrrolidin-2-yl)methyl)amino)methyl)-4H-pyrido[1,2-a]pyrimidin-8-yl)-[1,1′-biphenyl]-3-yl)-2,3-dihydro-1H-inden-2-yl)acetamide

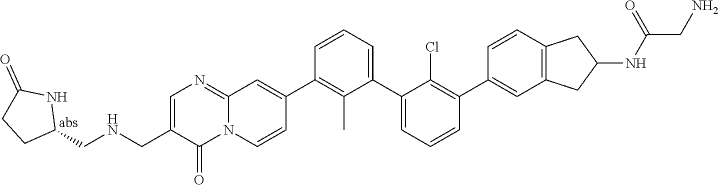 [SMILES: Cc1c(-c2ccn3c(=O)c(CNC[C@@H]4CCC(=O)N4)cnc3c2)cccc1-c1cccc(-c2ccc3c(c2)CC(NC(=O)CN)C3)c1Cl]

### 5-(3-Bromo-2-chloro-phenyl)indan-2-amine

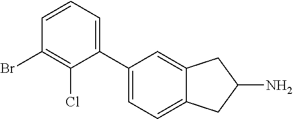 [SMILES: NC1Cc2ccc(-c3cccc(Br)c3Cl)cc2C1]

Tert-butyl N-[5-(3-bromo-2-chloro-phenyl)indan-2-yl]carbamate (0.50 g, 1.18 mmol) in DCM/TFA (2:1) mixture was stirred for 30 min, concentrated, neutralized, and purified by flash column chromatography to give 5-(3-bromo-2-chloro-phenyl)indan-2-amine (0.31 g, 81%). MS: m/z found 322.0 [M+H]⁺.

### tert-Butyl N-[[8-[3-[3-(2-aminoindan-5-yl)-2-chloro-phenyl]-2-methyl-phenyl]-4-oxo-pyrido[1,2-a]pyrimidin-3-yl]methyl]-N-[[(2S)-5-oxopyrrolidin-2-yl]methyl]carbamate

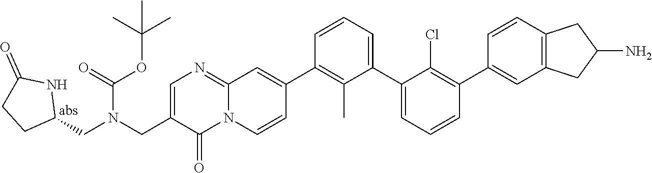 [SMILES: Cc1c(-c2ccn3c(=O)c(CN(C[C@@H]4CCC(=O)N4)C(=O)OC(C)(C)C)cnc3c2)cccc1-c1cccc(-c2ccc3c(c2)CC(N)C3)c1Cl]

To a solution of tert-butyl N-[[8-[2-methyl-3-(4,4,5,5-tetramethyl-1,3,2-dioxaborolan-2-yl)phenyl]-4-oxo-pyrido[1,2-a]pyrimidin-3-yl]methyl]-N-[[(2S)-5-oxopyrrolidin-2-yl]methyl]carbamate (0.15 g, 0.25 mmol), 5-(3-bromo-2-chloro-phenyl)indan-2-amine (0.11 g, 0.33 mmol) and potassium carbonate (0.11 g, 0.76 mmol) in degassed dioxane/water (5:1) was added Pd(PPh₃)₄ (29 mg, 0.03 mmol) and the mixture was stirred at 95° C. for 2 hours under N₂(g). Water was added and the mixture was extracted into EtOAc three times. The combined organic layers were dried over anhydrous sodium sulfate, concentrated, and purified on silica gel column using MeOH in DCM (10%) as eluent to give tert-butyl N-[[8-[3-[3-(2-aminoindan-5-yl)-2-chloro-phenyl]-2-methyl-phenyl]-4-oxo-pyrido[1,2-a]pyrimidin-3-yl]methyl]-N-[[(2S)-5-oxopyrrolidin-2-yl]methyl]carbamate (40 mg, 22.3%). MS: m/z found 704.4 [M+H]⁺.

### tert-Butyl N-[[8-[3-[3-[2-[[2-(tert-butoxycarbonylamino)acetyl]amino]indan-5-yl]-2-chloro-phenyl]-2-methyl-phenyl]-4-oxo-pyrido[1,2-a]pyrimidin-3-yl]methyl]-N-[[(2S)-5-oxopyrrolidin-2-yl]methyl]carbamate

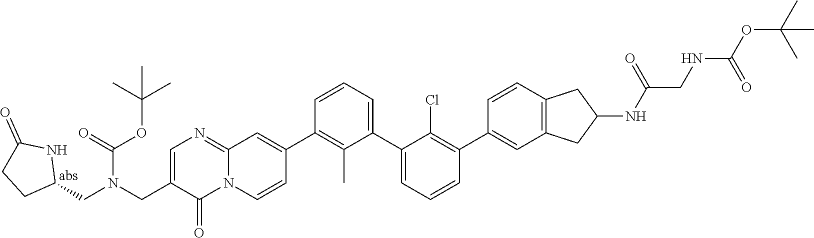 [SMILES: Cc1c(-c2ccn3c(=O)c(CN(C[C@@H]4CCC(=O)N4)C(=O)OC(C)(C)C)cnc3c2)cccc1-c1cccc(-c2ccc3c(c2)CC(NC(=O)CNC(=O)OC(C)(C)C)C3)c1Cl]

To a mixture of 2-(tert-butoxycarbonylamino)acetic acid (15 mg, 0.09 mmol) and HATU (32 mg, 0.09 mmol) in DMF was added DIPEA (15 uL, 11 mg, 0.09 mmol). The mixture was stirred for 10 minutes, tert-butyl N-[[8-[3-[3-(2-aminoindan-5-yl)-2-chloro-phenyl]-2-methyl-phenyl]-4-oxo-pyrido[1,2-a]pyrimidin-3-yl]methyl]-N-[[(2S)-5-oxopyrrolidin-2-yl]methyl]carbamate (30 mg, 0.04 mmol) was added and the reaction mixture was stirred overnight. Water was added and the residue was extracted into EtOAc two times. The organic layer was dried over anhydrous Na₂SO₄ and concentrated in vacuo. Purification with silica gel column using MeOH in DCM (0 to 10%) provided tert-butyl N-[[8-[3-[3-[2-[[2-(tert-butoxycarbonylamino)acetyl]amino]indan-5-yl]-2-chloro-phenyl]-2-methyl-phenyl]-4-oxo-pyrido[1,2-a]pyrimidin-3-yl]methyl]-N-[[(2S)-5-oxopyrrolidin-2-yl]methyl]carbamate (23.00 mg, 62.7%). MS: m/z found 861.5 [M+H]⁺.

### 2-Amino-N-[5-[2-chloro-3-[2-methyl-3-[4-oxo-3-[[[(2S)-5-oxopyrrolidin-2-yl]methylamino]methyl]pyrido[1,2-a]pyrimidin-8-yl]phenyl]phenyl]indan-2-yl]acetamide

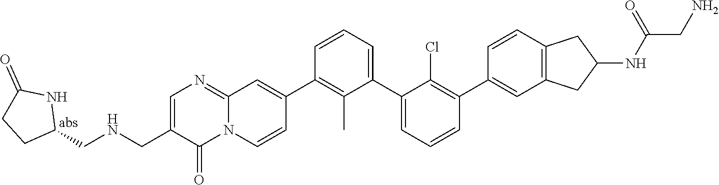 [SMILES: Cc1c(-c2ccn3c(=O)c(CNC[C@@H]4CCC(=O)N4)cnc3c2)cccc1-c1cccc(-c2ccc3c(c2)CC(NC(=O)CN)C3)c1Cl]

tert-Butyl N-[[8-[3-[3-[2-[[2-(tert-butoxycarbonylamino)acetyl]amino]indan-5-yl]-2-chloro-phenyl]-2-methyl-phenyl]-4-oxo-pyrido[1,2-a]pyrimidin-3-yl]methyl]-N-[[(2S)-5-oxopyrrolidin-2-yl]methyl]carbamate (20 mg, 0.02 mmol) was stirred in a 1:1 mixture of DCM/TFA, concentrated and subjected to reversed phase chromatography to give 2-amino-N-[5-[2-chloro-3-[2-methyl-3-[4-oxo-3-[[[(2S)-5-oxopyrrolidin-2-yl]methylamino]methyl]pyrido[1,2-a]pyrimidin-8-yl]phenyl]phenyl]indan-2-yl]acetamide (13 mg, 84.7%). MS: m/z found 661.4 [M+H]⁺; ¹H NMR (400 MHz, Methanol-d₄): δ 9.40 (d, J=7.1 Hz, 1H), 8.81 (s, 1H), 8.06 (d, J=1.6 Hz, 1H), 8.00 (dd, J=7.2, 1.7 Hz, 1H), 7.56-7.50 (m, 2H), 7.50-7.45 (m, 1H), 7.43-7.38 (m, 2H), 7.35-7.29 (m, 3H), 7.28-7.23 (m, 1H), 4.69 (tt, J=7.2, 5.2 Hz, 1H), 4.45 (d, J=2.0 Hz, 2H), 4.19-4.10 (m, 1H), 3.66 (s, 2H), 3.39-3.35 (m, 2H), 3.35-3.33 (m, 2H), 2.94 (dd, J=16.1, 5.2 Hz, 2H), 2.51-2.34 (m, 3H), 2.19 (s, 3H), 2.05-1.92 (m, 1H).

### Example 44: (5S,5'S)-5,5′-((((2,2′-Dichloro-[1,1′-biphenyl]-3,3′-diyl)bis(2,3-dihydrofuro[2,3-b]pyridine-6,3-diyl))bis(azanediyl))bis(methylene))bis(pyrrolidin-2-one)

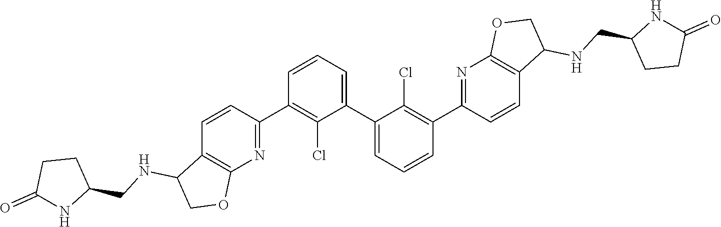 [SMILES: O=C1CC[C@@H](CNC2COc3nc(-c4cccc(-c5cccc(-c6ccc7c(n6)OCC7NC[C@@H]6CCC(=O)N6)c5Cl)c4Cl)ccc32)N1]

### Ethyl 6-chloro-3-oxo-2,3-dihydrofuro[2,3-b]pyridine-2-carboxylate

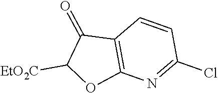 [SMILES: CCOC(=O)C1Oc2nc(Cl)ccc2C1=O]

To a solution of sodium hydride (20.2 g, 505 mmol) in DME (400 mL) was added ethyl 2-hydroxyacetate (50.5 g, 485 mmol) dropwise at 0° C. The reaction mixture was stirred at 0° C. for 0.5 h. Methyl 2,6-dichloronicotinate (40 g, 194 mmol) in DME (150 mL) was added to the mixture at r.t. The mixture was stirred at 75° C. for 2.5 hr. The reaction mixture was concentrated. The residue was treated with H₂O (100 mL) and EtOAc (50 mL). The mixture was filtered to give the cake. The crude product was washed with PE/EtOAc (50 mL/50 mL) and dried under reduced pressure. Ethyl 6-chloro-3-oxo-2,3-dihydrofuro[2,3-b]pyridine-2-carboxylate (43 g, crude) was obtained as a yellow solid. m/z found: 242 [M+H]⁺.

### 6-Chlorofuro[2,3-b]pyridin-3(2H)-one

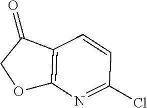 [SMILES: O=C1COc2nc(Cl)ccc21]

A mixture of ethyl 6-chloro-3-oxo-2,3-dihydrofuro[2,3-b]pyridine-2-carboxylate (6.5 g, 26.9 mmol) in concentrated HCl (18 mL) and dioxane (70 mL). The mixture was stirred at 95° C. for 12 hr. The reaction mixture was concentrated. The crude product was purified by reversed phase HPLC to give 6-chlorofuro[2,3-b]pyridin-3(2H)-one (1.6 g, 35% yield) as a yellow solid. ¹H NMR (400 MHz, DMSO-d₆): δ 8.18 (d, J=7.6 Hz, 1H), 7.34 (d, J=7.6 Hz, 1H), 4.94 (s, 2H).

### (5S)-5-(((6-Chloro-2,3-dihydrofuro[2,3-b]pyridin-3-yl)amino)methyl)pyrrolidin-2-one

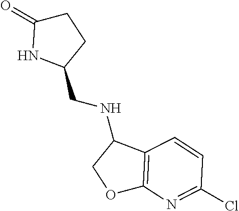 [SMILES: O=C1CC[C@@H](CNC2COc3nc(Cl)ccc32)N1]

To a solution of 6-chlorofuro[2,3-b]pyridin-3(2H)-one (800 mg, 4.72 mmol) and (S)-5-(aminomethyl)pyrrolidin-2-one (1.08 g, 9.44 mmol) in MeOH (10 mL) was added glacial HOAc (0.27 mL, 4.72 mmol), The mixture was stirred at 40° C. for 6 hr. Sodium cyanoborohydride (889 mg, 14.2 mmol) was added to the mixture. The mixture was stirred at 80° C. for 12 hr. The reaction mixture was concentrated. The residue was purified by normal phase SiO₂ chromatography (0˜30% MeOH/Ethyl acetate) to give (5S)-5-(((6-chloro-2,3-dihydrofuro[2,3-b]pyridin-3-yl)amino)methyl)pyrrolidin-2-one (230 mg, 18% yield) as a yellow solid. ¹H NMR (400 MHz, CDCl₃): δ7.50 (d, J=7.6 Hz, 1H), 6.84 (dd, J=7.6 Hz, J=2.8 Hz, 1H), 6.12 (d, J=10 Hz, 1H), 4.63-4.58 (m, 1H), 4.47-4.42 (m, 1H), 4.37-4.34 (m, 1H), 3.65-3.56 (m, 1H), 2.85-2.70 (m, 1H), 2.47-2.39 (m, 1H), 2.32-2.27 (m, 2H), 2.17-2.15 (m, 1H), 1.71-1.69 (m, 1H).

### (5S,5'S)-5,5′-(((6,6′-(2,2′-Dichloro-[1,1′-biphenyl]-3,3′-diyl)bis(2,3-dihydrofuro[2,3-b]pyridine-6,3-diyl))bis(azanediyl))bis(methylene))bis(pyrrolidin-2-one)

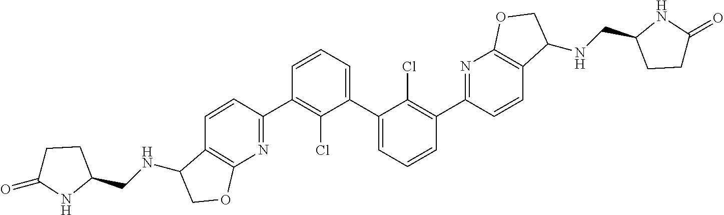 [SMILES: O=C1CC[C@@H](CNC2COc3nc(-c4cccc(-c5cccc(-c6ccc7c(n6)OCC7NC[C@@H]6CCC(=O)N6)c5Cl)c4Cl)ccc32)N1]

To a solution of (5S)-5-(((6-chloro-2,3-dihydrofuro[2,3-b]pyridin-3-yl)amino)methyl)pyrrolidin-2-one (230 mg, 0.86 mmol) and 2,2′-(2,2′-dichloro-[1,1′-biphenyl]-3,3′-diyl)bis(4,4,5,5-tetramethyl-1,3,2-dioxaborolane) (500 mg, 0.42 mmol) in toluene (10 mL) and H₂O (1 mL) was added potassium phosphate (357.49 mg, 1.68 mmol), dicyclohexyl(2′,6′-dimethoxy-[1,1′-biphenyl]-2-yl)phosphine (34.6 mg, 0.084 mmol) and tris(dibenzylideneacetone)dipalladium (38.5 mg, 0.042 mmol). The mixture was stirred at 110° C. for 4 hr. The reaction mixture was concentrated. The residue was purified by normal phase SiO₂ chromatography (10-100% MeOH/Ethyl acetate) to give 60 mg of crude product. The crude product was purified by reverse phase HPLC to give (5S,5'S)-5,5′-(((6,6′-(2,2′-dichloro-[1,1′-biphenyl]-3,3′-diyl)bis(2,3-dihydrofuro[2,3-b]pyridine-6,3-diyl))bis(azanediyl))bis(methylene))bis(pyrrolidin-2-one) (25.25 mg) as a white solid. m/z found: 685 [M+H]⁺. ¹H NMR (400 MHz, Methanol-d₄) δ 7.94 (d, J=7.6 Hz, 2H), 7.52-7.50 (m, 4H), 7.41 (dd, J=7.2 Hz, J=2.4 Hz, 2H), 7.20 (s, J=7.6 Hz, 2H), 4.75-4.22 (m, 2H), 4.67-4.64 (m, 2H), 4.54-4.50 (m, 2H), 3.82-3.76 (m, 2H), 2.89-2.85 (m, 1H), 2.79-2.77 (m, 2H), 2.71-2.67 (m, 1H), 2.39-2.24 (m, 6H), 1.92-1.82 (m, 2H).

### Example 45: (2S)-2-Amino-N-(5-(2-chloro-2′-methyl-3′-(4-oxo-3-(((((S)-5-oxopyrrolidin-2-yl)methyl)amino)methyl)-4H-pyrido[1,2-a]pyrimidin-8-yl)-[1,1′-biphenyl]-3-yl)-2,3-dihydro-1H-inden-2-yl)-3-hydroxypropanamide

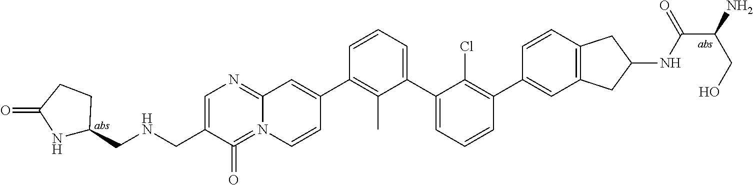

### tert-Butyl N-[[8-[3-[3-[2-[[(2S)-2-(tert-butoxycarbonylamino)-3-hydroxy-propanoyl]amino]indan-5-yl]-2-chloro-phenyl]-2-methyl-phenyl]-4-oxo-pyrido[1,2-a]pyrimidin-3-yl]methyl]-N-[[(2S)-5-oxopyrrolidin-2-yl]methyl]carbamate

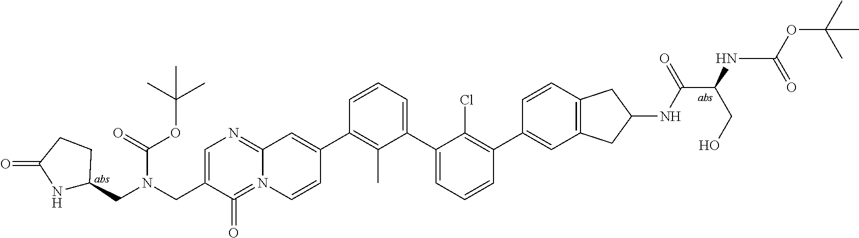

To a mixture of (2S)-2-(tert-butoxycarbonylamino)-3-hydroxy-propanoic acid (17.48 mg, 0.09 mmol) and HATU (32.39 mg, 0.09 mmol) in DMF was added DIPEA (15 uL, 11 mg, 0.09 mmol). The mixture was stirred for 10 minutes, tert-butyl N-[[8-[3-[3-(2-aminoindan-5-yl)-2-chloro-phenyl]-2-methyl-phenyl]-4-oxo-pyrido[1,2-a]pyrimidin-3-yl]methyl]-N-[[(2S)-5-oxopyrrolidin-2-yl]methyl]carbamate (30 mg, 0.04 mmol) was added and the reaction mixture was stirred overnight. Water was added and the residue was extracted into EtOAc two times. The organic layer was dried over anhydrous Na₂SO₄ and concentrated in vacuo. Purification by HPLC provided tert-butyl N-[[8-[3-[3-[2-[[(2S)-2-(tert-butoxycarbonylamino)-3-hydroxy-propanoyl]amino]indan-5-yl]-2-chloro-phenyl]-2-methyl-phenyl]-4-oxo-pyrido[1,2-a]pyrimidin-3-yl]methyl]-N-[[(2S)-5-oxopyrrolidin-2-yl]methyl]carbamate (20 mg, 53%). MS: m/z found 891.4 [M+H]⁺.

### (2S)-2-Amino-N-[5-[2-chloro-3-[2-methyl-3-[4-oxo-3-[[[(2S)-5-oxopyrrolidin-2-yl]methylamino]methyl]pyrido[1,2-a]pyrimidin-8-yl]phenyl]phenyl]indan-2-yl]-3-hydroxy-propanamide

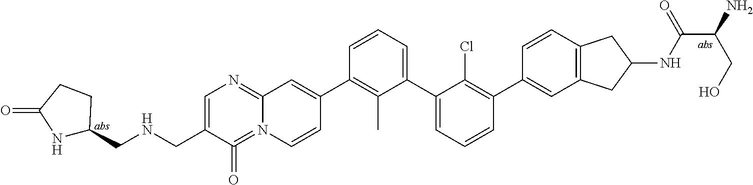

tert-Butyl N-[[8-[3-[3-[2-[[(2S)-2-(tert-butoxycarbonylamino)-3-hydroxy-propanoyl]amino]indan-5-yl]-2-chloro-phenyl]-2-methyl-phenyl]-4-oxo-pyrido[1,2-a]pyrimidin-3-yl]methyl]-N-[[(2S)-5-oxopyrrolidin-2-yl]methyl]carbamate (20 mg, 0.02 mmol) was stirred in a 1:1 mixture of DCM/TFA for 30 min, concentrated, and purified by HPLC to give (2S)-2-amino-N-[5-[2-chloro-3-[2-methyl-3-[4-oxo-3-[[[(2S)-5-oxopyrrolidin-2-yl]methylamino]methyl]pyrido[1,2-a]pyrimidin-8-yl]phenyl]phenyl]indan-2-yl]-3-hydroxy-propanamide (13 mg, 83.8%). MS: m/z found 691.4 [M+H]⁺; ¹H NMR (400 MHz, Methanol-d₄): δ 9.39 (d, J=7.2 Hz, 1H), 8.80 (s, 1H), 8.05 (d, J=1.7 Hz, 1H), 8.00 (dd, J=7.2, 1.8 Hz, 1H), 7.57-7.44 (m, 3H), 7.41 (tt, J=5.5, 2.6 Hz, 2H), 7.32 (dq, J=5.6, 1.9 Hz, 3H), 7.25 (dd, J=7.7, 1.6 Hz, 1H), 4.69 (tt, J=7.3, 5.3 Hz, 1H), 4.44 (d, J=2.2 Hz, 2H), 4.14 (td, J=7.3, 4.0 Hz, 1H), 3.95-3.86 (m, 2H), 3.80 (ddd, J=12.7, 8.0, 1.4 Hz, 1H), 3.41-3.35 (m, 2H), 3.35-3.32 (m, 2H), 2.96 (dt, J=15.7, 7.3 Hz, 2H), 2.50-2.33 (m, 3H), 2.19 (s, 3H), 2.04-1.90 (m, 1H).

### Example 46: (2S)—N-(5-(2-Chloro-2′-methyl-3′-(4-oxo-3-(((((S)-5-oxopyrrolidin-2-yl)methyl)amino)methyl)-4H-pyrido[1,2-a]pyrimidin-8-yl)-[1,1′-biphenyl]-3-yl)-2,3-dihydro-1H-inden-2-yl)pyrrolidine-2-carboxamide

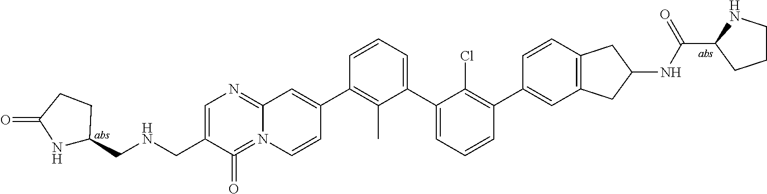

### tert-Butyl (2S)-2-[[5-[3-[3-[3-[[tert-butoxycarbonyl-[[(2S)-5-oxopyrrolidin-2-yl]methyl]amino]methyl]-4-oxo-pyrido[1,2-a]pyrimidin-8-yl]-2-methyl-phenyl]-2-chloro-phenyl]indan-2-yl]carbamoyl]pyrrolidine-1-carboxylate

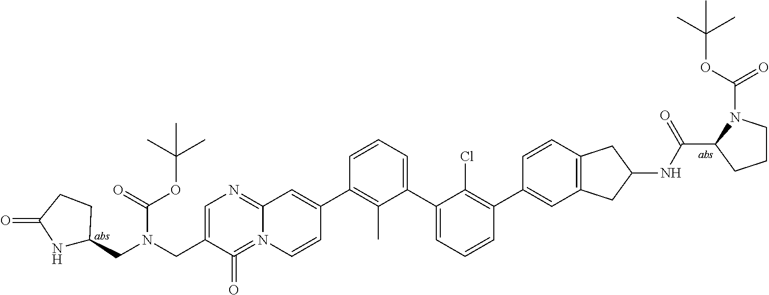

To a mixture of (2S)-1-tert-butoxycarbonylpyrrolidine-2-carboxylic acid (18 mg, 0.09 mmol) and HATU (32 mg, 0.09 mmol) in DMF was added DIPEA (15 uL, 11 mg, 0.09 mmol). The mixture was stirred for 10 minutes, tert-butyl N-[[8-[3-[3-(2-aminoindan-5-yl)-2-chloro-phenyl]-2-methyl-phenyl]-4-oxo-pyrido[1,2-a]pyrimidin-3-yl]methyl]-N-[[(2S)-5-oxopyrrolidin-2-yl]methyl]carbamate (30 mg, 0.04 mmol) was added and the reaction mixture was stirred overnight. Water was added and the residue was extracted into EtOAc two times. The organic layer was dried over anhydrous Na₂SO₄ and concentrated in vacuo. Purification by HPLC provided tert-butyl (2S)-2-[[5-[3-[3-[3-[[tert-butoxycarbonyl-[[(2S)-5-oxopyrrolidin-2-yl]methyl]amino]methyl]-4-oxo-pyrido[1,2-a]pyrimidin-8-yl]-2-methyl-phenyl]-2-chloro-phenyl]indan-2-yl]carbamoyl]pyrrolidine-1-carboxylate (30 mg, 78.1%). MS: m/z found 901.5 [M+H]⁺.

### (2S)-N-[5-[2-Chloro-3-[2-methyl-3-[4-oxo-3-[[[(2S)-5-oxopyrrolidin-2-yl]methylamino]methyl]pyrido[1,2-a]pyrimidin-8-yl]phenyl]phenyl]indan-2-yl]pyrrolidine-2-carboxamide

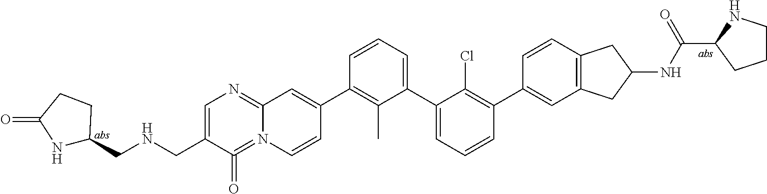

tert-Butyl (2S)-2-[[5-[3-[3-[3-[[tert-butoxycarbonyl-[[(2S)-5-oxopyrrolidin-2-yl]methyl]amino]methyl]-4-oxo-pyrido[1,2-a]pyrimidin-8-yl]-2-methyl-phenyl]-2-chloro-phenyl]indan-2-yl]carbamoyl]pyrrolidine-1-carboxylate (30 mg, 0.03 mmol) was stirred in a 1:1 mixture of DCM/TFA for 30 min, concentrated, and purified by HPLC to give (2S)-N-[5-[2-chloro-3-[2-methyl-3-[4-oxo-3-[[[(2S)-5-oxopyrrolidin-2-yl]methylamino]methyl]pyrido[1,2-a]pyrimidin-8-yl]phenyl]phenyl]indan-2-yl]pyrrolidine-2-carboxamide (21 mg, 90.0%). MS: m/z found 701.5 [M+H]⁺; 1 H NMR (400 MHz, Methanol-d₄): δ 9.40 (dt, J=7.1, 0.7 Hz, 1H), 8.81 (s, 1H), 8.07 (dd, J=1.7, 0.8 Hz, 1H), 8.00 (ddd, J=7.2, 1.8, 0.7 Hz, 1H), 7.57-7.45 (m, 3H), 7.43-7.37 (m, 2H), 7.35-7.29 (m, 3H), 7.25 (dd, J=7.7, 1.7 Hz, 1H), 4.69 (tt, J=7.1, 5.2 Hz, 1H), 4.45 (d, J=2.0 Hz, 2H), 4.23 (dd, J=8.5, 6.9 Hz, 1H), 4.20-4.11 (m, 1H), 3.47-3.35 (m, 4H), 3.34-3.30 (m, 2H), 2.95 (td, J=16.6, 5.3 Hz, 2H), 2.54-2.33 (m, 4H), 2.19 (s, 3H), 2.13-1.93 (m, 4H).

### Example 47: 8-(2,2′-Dichloro-3′-(6-methoxy-5-(((((S)-5-oxopyrrolidin-2-yl)methyl)amino)methyl)pyrazin-2-yl)-[1,1′-biphenyl]-3-yl)-3-(((((S)-5-oxopyrrolidin-2-yl)methyl)amino)methyl)-4H-pyrido[1,2-a]pyrimidin-4-one

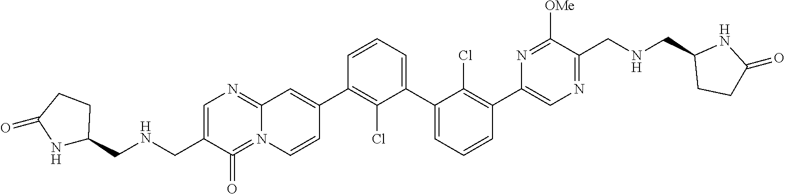

### (S)-8-(3-Bromo-2-chlorophenyl)-3-((((5-oxopyrrolidin-2-yl)methyl)amino)methyl)-4H-pyrido[1,2-a]pyrimidin-4-one

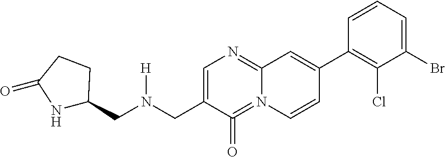

To a mixture of 8-(3-bromo-2-chloro-phenyl)-4-oxo-pyrido[1,2-a]pyrimidine-3-carbaldehyde (2 g, 5.5 mmol) in THE (15 mL) was added glacial acetic acid (0.31 mL, 5.5 mmol) and 4 Å molecular sieve (0.05 g). Then (S)-5-(aminomethyl)pyrrolidin-2-one as HCl salt (1 g, 6.6 mmol) and triethylamine (0.92 mL, 6.6 mmol) in MeOH (15 mL) was added to the mixture and stirred at r.t. for 2 hr under N₂. Then sodium cyanoborohydride (1 g, 16.5 mmol) was added to the mixture and stirred at r.t. for 0.5 hr under N₂. The mixture was concentrated to afford (S)-8-(3-bromo-2-chlorophenyl)-3-((((5-oxopyrrolidin-2-yl)methyl)amino)methyl)-4H-pyrido[1,2-a]pyrimidin-4-one in THF/MeOH mixture (1:1, 30 mL) as a brown suspensions without purification. m/z found: 463 [M+H]⁺.

### (S)-tert-Butyl((8-(3-bromo-2-chlorophenyl)-4-oxo-4H-pyrido[1,2-a]pyrimidin-3-yl)methyl)((5-oxopyrrolidin-2-yl)methyl)carbamate

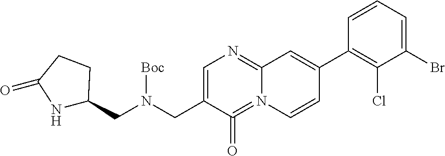

To a mixture of (S)-8-(3-bromo-2-chlorophenyl)-3-((((5-oxopyrrolidin-2-yl)methyl)amino)methyl)-4H-pyrido[1,2-a]pyrimidin-4-one (5.50 mmol) in THF/MeOH mixture (1:1, 30 mL) was added di-tert-butyl dicarbonate (5 mL, 22 mmol) and triethylamine (2.3 mL, 16.5 mmol). The mixture was stirred at r.t. for 1 hr under N₂. The mixture was concentrated to give a crude residue that was purified by reverse phase HPLC to afford (S)-tert-butyl((8-(3-bromo-2-chlorophenyl)-4-oxo-4H-pyrido[1,2-a]pyrimidin-3-yl)methyl)((5-oxopyrrolidin-2-yl)methyl)carbamate (0.6 g, 19% yield) as a yellow solid. m/z found: 563 [M+H]⁺.

### tert-Butyl N-[[8-[3-[3-[5-[[tert-butoxycarbonyl-[[(2S)-5-oxopyrrolidin-2-yl]methyl]amino]methyl]-6-methoxy-pyrazin-2-yl]-2-chloro-phenyl]-2-chloro-phenyl]-4-oxo-pyrido[1,2-a]pyrimidin-3-yl]methyl]-N-[[(2S)-5-oxopyrrolidin-2-yl]methyl]carbamate

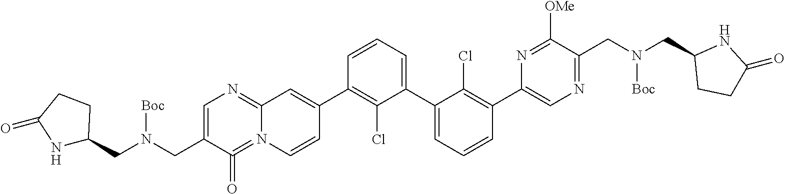

To a mixture of (S)-tert-butyl (S)-tert-butyl ((8-(3-bromo-2-chlorophenyl)-4-oxo-4H-pyrido[1,2-a]pyrimidin-3-yl)methyl)((5-oxopyrrolidin-2-yl)methyl)carbamate (0.37 g, 0.66 mmol) and (S)-tert-butyl ((5-(2-chloro-3-(4,4,5,5-tetramethyl-1,3,2-dioxaborolan-2-yl)phenyl)-3-methoxypyrazin-2-yl)methyl)((5-oxopyrrolidin-2-yl)methyl)carbamate (0.38 g, 0.66 mmol) in THF/H₂O mixture (5:1, 12 mL) was added potassium phosphate (0.42 g, 2 mmol) and ditert-butyl(cyclopentyl)phosphane;dichloropalladium;iron (43.2 mg, 0.06 mmol). The mixture was stirred at 80° C. for 4 hr under N₂. The mixture was concentrated to give the residue. The residue was purified by normal phase SiO₂ chromatography (0-100% EtOAc/petroleum ether) to afford tert-butyl N-[[8-[3-[3-[5-[[tert-butoxycarbonyl-[[(2S)-5-oxopyrrolidin-2-yl]methyl]amino]methyl]-6-methoxy-pyrazin-2-yl]-2-chloro-phenyl]-2-chloro-phenyl]-4-oxo-pyrido[1,2-a]pyrimidin-3-yl]methyl]-N-[[(2S)-5-oxopyrrolidin-2-yl]methyl]carbamate (450 mg, crude) as a brown solid. m/z found: 927 [M+H]⁺.

### 8-(2,2′-Dichloro-3′-(6-methoxy-5-(((((S)-5-oxopyrrolidin-2-yl)methyl)amino)methyl)pyrazin-2-yl)-[1,1′-biphenyl]-3-yl)-3-(((((S)-5-oxopyrrolidin-2-yl)methyl)amino)methyl)-4H-pyrido[1,2-a]pyrimidin-4-one

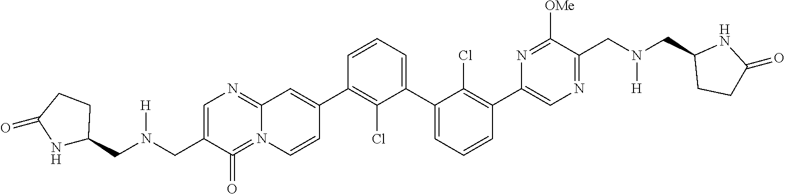

To a mixture of tert-butyl N-[[8-[3-[3-[5-[[tert-butoxycarbonyl-[[(2S)-5-oxopyrrolidin-2-yl]methyl]amino]methyl]-6-methoxy-pyrazin-2-yl]-2-chloro-phenyl]-2-chloro-phenyl]-4-oxo-pyrido[1,2-a]pyrimidin-3-yl]methyl]-N-[[(2S)-5-oxopyrrolidin-2-yl]methyl]carbamate (0.35 g, 0.37 mmol) in DCM (30 mL) was added trifluoroacetic acid (10.5 mL, 140 mmol). The mixture was stirred at r.t. for 1 hr under N₂. The mixture was combined with another batch at 0.1 g scale. The combined mixture was concentrated to give the residue. The residue was purified by reverse phase HPLC to afford 8-(2,2′-dichloro-3′-(6-methoxy-5-(((((S)-5-oxopyrrolidin-2-yl)methyl)amino)methyl)pyrazin-2-yl)-[1,1′-biphenyl]-3-yl)-3-(((((S)-5-oxopyrrolidin-2-yl)methyl)amino)methyl)-4H-pyrido[1,2-a]pyrimidin-4-one (63.05 mg) as a white solid. m/z: 727 [M+H]⁺. ¹H NMR (400 MHz, Methanol-d₄): δ 9.15 (d, J=7.2 Hz, 1H), 8.45-8.44 (m, 2H), 7.80 (d, J=1.2 Hz, 1H), 7.71-7.70 (m, 1H), 7.63-7.41 (m, 6H), 4.15 (d, J=2.4 Hz, 2H), 4.08 (s, 3H), 4.05 (d, J=4 Hz, 2H), 3.94-3.89 (m, 2H), 2.97-2.87 (m, 4H), 2.38-2.29 (m, 6H), 1.88-1.83 (m, 2H).

### Example 48: (S)-8,8′-(2,2′-Dichloro-[1,1′-biphenyl]-3,3′-diyl)bis(3-(((((S)-5-oxopyrrolidin-2-yl)methyl)amino)methyl)-4H-pyrido[1,2-a]pyrimidin-4-one)

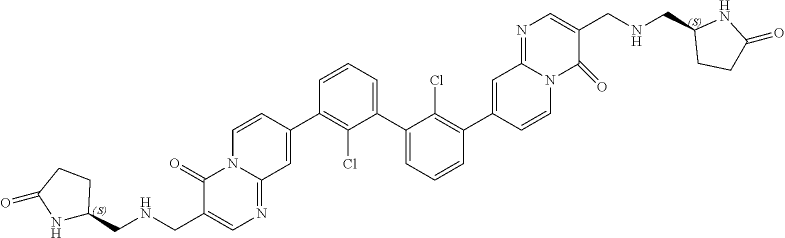

### 8-(3-Bromo-2-chlorophenyl)-4-oxo-4H-pyrido[1,2-a]pyrimidine-3-carbaldehyde

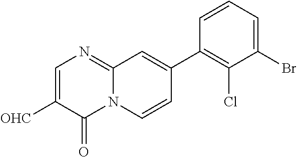 [SMILES: O=Cc1cnc2cc(-c3cccc(Br)c3Cl)ccn2c1=O]

A mixture of 8-bromo-4-oxo-4H-pyrido[1,2-a]pyrimidine-3-carbaldehyde (5 g, 19.7 mmol), 2-(3-bromo-2-chlorophenyl)-4,4,5,5-tetramethyl-1,3,2-dioxaborolane (5 g, 15.8 mmol), potassium carbonate (8.2 g, 59.3 mmol) and [1,1-Bis(diphenylphosphino)ferrocene]palladium(II) chloride dichloromethane complex (1.6 g, 2 mmol) in THF/H₂O mixture (5:1, 120 mL) was stirred at 80° C. for 3 h under N₂. The mixture was concentrated and the residue was triturated with ethyl acetate (30 mL) at r.t. for 0.5 h to afford 8-(3-bromo-2-chloro-phenyl)-4-oxo-pyrido[1,2-a]pyrimidine-3-carbaldehyde as a brown solid (5.5 g, 76% yield). ¹H NMR (400 MHz, DMSO-d₆): δ 10.20 (s, 1H), 9.27 (d, J=7.2 Hz, 1H), 8.79 (s, 1H), 8.02 (s, 1H), 7.98 (d, J=8 Hz, 1H), 7.76 (dd, J₁=1.2 Hz, J₂=7.2 Hz 1H), 7.63-7.61 (m, 1H), 7.51-7.49 (m, 1H).

### (S)-8-(3-Bromo-2-chlorophenyl)-3-((((5-oxopyrrolidin-2-yl)methyl)amino)methyl)-4H-pyrido[1,2-a]pyrimidin-4-one

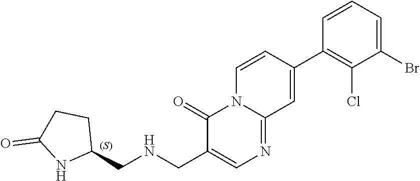

To a mixture of (S)-5-(aminomethyl)pyrrolidin-2-one as a HCl salt (0.5 g, 3.3 mmol) in MeOH (3 mL) was added triethylamine (0.5 mL, 3.3 mmol) in one portion. This mixture was added to a mixture of 8-(3-bromo-2-chlorophenyl)-4-oxo-4H-pyrido[1,2-a]pyrimidine-3-carbaldehyde (1 g, 2.75 mmol) and glacial acetic acid (0.16 mL, 2.75 mmol) in THE (20 mL). The reaction mixture was stirred at r.t. for 16 h. To the mixture was added sodium triacetoxyborohydride (0.52 g, 8.25 mmol). The mixture was stirred at r.t. for 0.5 h. (S)-8-(3-bromo-2-chlorophenyl)-3-((((5-oxopyrrolidin-2-yl)methyl)amino)methyl)-4H-pyrido[1,2-a]pyrimidin-4-one in a solution of THF/MeOH (brown liquid, 4:1, 25 mL) was obtained. m/z: 463 [M+H]⁺. The mixture was used for next step without work-up and purification.

### (S)-tert-Butyl ((8-(3-bromo-2-chlorophenyl)-4-oxo-4H-pyrido[1,2-a]pyrimidin-3-yl)methyl)((5-oxopyrrolidin-2-yl)methyl)carbamate

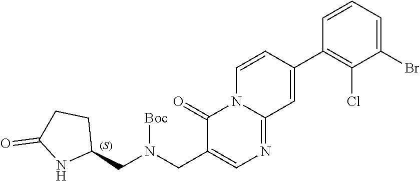

To a mixture of (S)-8-(3-bromo-2-chlorophenyl)-3-((((5-oxopyrrolidin-2-yl)methyl)amino)methyl)-4H-pyrido[1,2-a]pyrimidin-4-one (1.27 g, 2.75 mmol) in a THF/MeOH (brown liquid, 4:1, 25 mL) suspension and di-tert-butyl dicarbonate (0.8 mL, 3.58 mmol) was added triethylamine (1.2 mL, 8.25 mmol) under N₂. The mixture was stirred at r.t. for 1 h. The reaction mixture was concentrated under reduced pressure. The residue was purified by normal phase SiO₂ chromatography (30˜100% ethyl acetate/30% THF in petroleum ether) to afford 1.9 g of crude product. The crude product was purified by reverse phase HPLC to afford (S)-tert-butyl ((8-(3-bromo-2-chlorophenyl)-4-oxo-4H-pyrido[1,2-a]pyrimidin-3-yl)methyl)((5-oxopyrrolidin-2-yl)methyl)carbamate as a yellow solid (0.60 g, 38% yield). ¹H NMR (400 MHz, DMSO-d₆): δ 9.02 (d, J=7.2 Hz, 1H), 8.24 (s, 1H), 7.94 (dd, J₁=1.6 Hz, J₂=8 Hz, 1H), 7.77 (d, J=7.2 Hz, 2H), 7.59 (d, J=6.4 Hz, 1H), 7.49-7.44 (m, 2H), 4.43-4.33 (m, 2H), 3.80 (br s, 1H), 3.38 (br s, 1H), 3.17 (d, J=5.2 Hz, 1H), 2.19-2.02 (m, 3H), 1.72 (br s, 1H), 1.40 (s, 9H).

### (S)-tert-Butyl ((8-(2-chloro-3-(4,4,5,5-tetramethyl-1,3,2-dioxaborolan-2-yl)phenyl)-4-oxo-4H-pyrido[1,2-a]pyrimidin-3-yl)methyl)((5-oxopyrrolidin-2-yl)methyl)carbamate

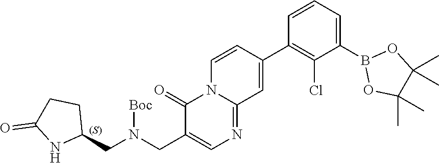

To a solution of (S)-tert-butyl ((8-(3-bromo-2-chlorophenyl)-4-oxo-4H-pyrido[1,2-a]pyrimidin-3-yl)methyl)((5-oxopyrrolidin-2-yl)methyl)carbamate (0.35 g, 0.62 mmol), 4,4,4′,4′,5,5,5′,5′-octamethyl-2,2′-bi(1,3,2-dioxaborolane) (0.32 g, 1.25 mmol) and potassium acetate (0.18 g, 1.87 mmol) in a dioxane/THF mixture (1:1, 6 mL) was added 1,1′-bis(diphenylphosphino)ferrocene-palladium(ii) dichloride dichloromethane complex (0.05 g, 0.06 mmol) under N₂. The mixture was stirred at 110° C. for 1 h. The mixture was poured into water (2 mL) and the aqueous phase was extracted with ethyl acetate (3×3 mL). The combined organic phase was washed with saturated aqueous brine solution (2×2 mL), dried with anhydrous sodium sulfate, filtered and concentrated under vacuum. The residue was purified by normal phase SiO₂ chromatography (0-100% ethyl acetate/petroleum ether) to afford (S)-tert-butyl ((8-(2-chloro-3-(4,4,5,5-tetramethyl-1,3,2-dioxaborolan-2-yl)phenyl)-4-oxo-4H-pyrido[1,2-a]pyrimidin-3-yl)methyl)((5-oxopyrrolidin-2-yl)methyl)carbamate as a yellow oil (0.37 g, 45.5% yield). m/z found: 609 [M+H]⁺.

### Di-tert-butyl ((8,8′-(2,2′-dichloro-[1,1′-biphenyl]-3,3′-diyl)bis(4-oxo-4H-pyrido[1,2-a]pyrimidine-8,3-diyl))bis(methylene))bis((((S)-5-oxopyrrolidin-2-yl)methyl)carbamate)

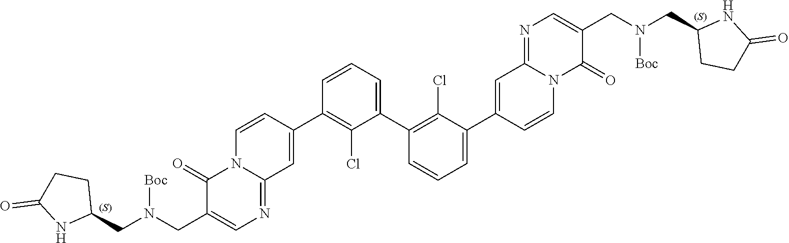

To a mixture of (S)-tert-butyl ((8-(3-bromo-2-chlorophenyl)-4-oxo-4H-pyrido[1,2-a]pyrimidin-3-yl)methyl)((5-oxopyrrolidin-2-yl)methyl)carbamate (0.15 g, 0.27 mmol), (S)-tert-butyl ((8-(2-chloro-3-(4,4,5,5-tetramethyl-1,3,2-dioxaborolan-2-yl)phenyl)-4-oxo-4H-pyrido[1,2-a]pyrimidin-3-yl)methyl)((5-oxopyrrolidin-2-yl)methyl)carbamate (0.35 g, 0.27 mmol) and potassium phosphate (0.17 g, 0.81 mmol) in a THF/H₂O mixture (4:1, 5 mL) was added di-tert-butyl(cyclopentyl)phosphane;dichloropalladium;iron (17.5 mg, 0.03 mmol) under N₂. The mixture was stirred at 80° C. for 1 h. To the mixture was added water (5 mL). The mixture was extracted with ethyl acetate (2×5 mL). The combined organic phase was washed with saturated aqueous brine solution (2×3 mL), dried with anhydrous sodium sulfate, filtered and concentrated to give di-tert-butyl ((8,8′-(2,2′-dichloro-[1,1′-biphenyl]-3,3′-diyl)bis(4-oxo-4H-pyrido[1,2-a]pyrimidine-8,3-diyl))bis(methylene))bis((((S)-5-oxopyrrolidin-2-yl)methyl)carbamate) as a yellow solid, which was used in the next step without further purification (0.31 g, crude). m/z found: 963 [M+H]⁺.

### (S)-8,8′-(2,2′-Dichloro-[1,1′-biphenyl]-3,3′-diyl)bis(3-(((((S)-5-oxopyrrolidin-2-yl)methyl)amino)methyl)-4H-pyrido[1,2-a]pyrimidin-4-one)

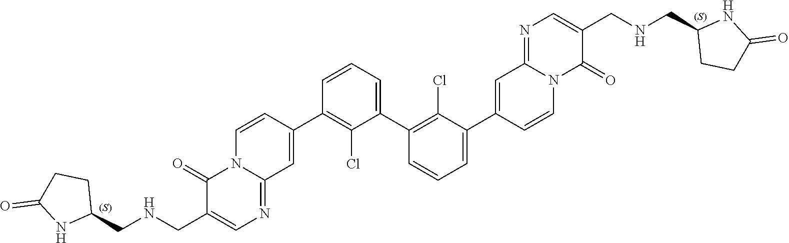

A crude mixture of di-tert-butyl ((8,8′-(2,2′-dichloro-[1,1′-biphenyl]-3,3′-diyl)bis(4-oxo-4H-pyrido[1,2-a]pyrimidine-8,3-diyl))bis(methylene))bis((((S)-5-oxopyrrolidin-2-yl)methyl)carbamate) (0.3 g, 0.31 mmol) and triethylamine (2 mL) in CH₂Cl₂ (5 mL) was stirred at r.t. for 30 min under N₂. The mixture was concentrated. The residue was purified by reverse phase HPLC to afford (S)-8,8′-(2,2′-dichloro-[1,1′-biphenyl]-3,3′-diyl)bis(3-(((((S)-5-oxopyrrolidin-2-yl)methyl)amino)methyl)-4H-pyrido[1,2-a]pyrimidin-4-one) as a white solid. (33.89 mg, 13% yield). m/z found: 765 [M+H]⁺. ¹H NMR (400 MHz, Methanol-d₄): δ 9.11 (d, J=7.6 Hz, 2H), 8.40 (s, 2H), 7.75 (d, J=1.2 Hz, 2H), 7.66-7.59 (m, 4H), 7.54-7.50 (m, 4H), 3.90-3.82 (m, 6H), 2.78-2.67 (m, 4H), 2.40-2.23 (m, 6H), 1.87-1.78 (m, 2H).

### Example 49: 8-(2′-Chloro-3′-(6-methoxy-5-(((((S)-5-oxopyrrolidin-2-yl)methyl)amino)methyl)pyrazin-2-yl)-2-methyl-[1,1′-biphenyl]-3-yl)-3-(((((S)-5-oxopyrrolidin-2-yl)methyl)amino)methyl)-4H-quinolizin-4-one

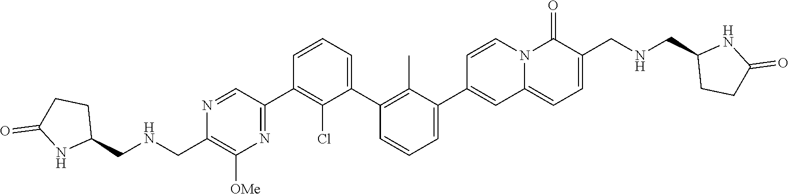 [SMILES: COc1nc(-c2cccc(-c3cccc(-c4ccn5c(=O)c(CNC[C@@H]6CCC(=O)N6)ccc5c4)c3C)c2Cl)cnc1CNC[C@@H]1CCC(=O)N1]

### (S)-8-(3-Bromo-2-methylphenyl)-3-((((5-oxopyrrolidin-2-yl)methyl)amino)methyl)-4H-quinolizin-4-one

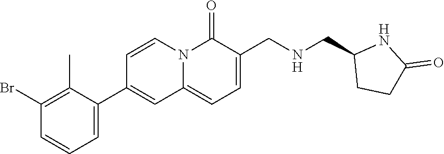 [SMILES: Cc1c(Br)cccc1-c1ccn2c(=O)c(CNC[C@@H]3CCC(=O)N3)ccc2c1]

To a mixture of 8-(3-bromo-2-methylphenyl)-4-oxo-4H-quinolizine-3-carbaldehyde (0.9 g, 2.63 mmol) and (5S)-5-(aminomethyl)pyrrolidin-2-one as a HCl salt (1.2 g, 8 mmol) in DCM (40 mL) was added TEA (1.8 mL, 13 mmol) and 4 Å molecular sieves (0.05 g). The mixture was stirred at 25° C. for 12 h. Sodium cyanoborohydride (0.8 g, 13 mmol) was added to the mixture and stirred for 30 min. The mixture was used directly for the next step. m/z found: 440 [M]⁺, 442 [M+2]⁺.

### (S)-tert-Butyl ((8-(3-bromo-2-methylphenyl)-4-oxo-4H-quinolizin-3-yl)methyl)((5-oxopyrrolidin-2-yl)methyl)carbamate

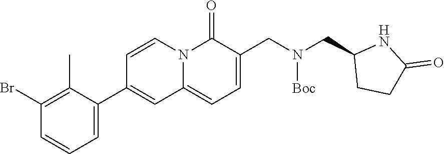 [SMILES: Cc1c(Br)cccc1-c1ccn2c(=O)c(CN(C[C@@H]3CCC(=O)N3)C(=O)OC(C)(C)C)ccc2c1]

To 8-(3-bromo-2-methyl-phenyl)-3-[[[(2S)-5-oxopyrrolidin-2-yl]methylamino]methyl]quinolizin-4-one (reaction mixture) was added bis(tert-butoxycarbonyl)oxide (1.5 g, 6.8 mmol). The mixture was stirred at r.t. for 1 hour. The reaction mixture was treated with water (30 mL) and extracted with EtOAc (50 mL×3). The combined organic layers were concentrated under reduced pressure. The residue was purified by normal phase SiO₂ chromatography (10-100% EtOAc/petroleum ether) to afford (S)-tert-butyl ((8-(3-bromo-2-methylphenyl)-4-oxo-4H-quinolizin-3-yl)methyl)((5-oxopyrrolidin-2-yl)methyl)carbamate (1.5 g) as yellow solid. The product was purified again by reverse phase HPLC to afford (S)-tert-butyl ((8-(3-bromo-2-methylphenyl)-4-oxo-4H-quinolizin-3-yl)methyl)((5-oxopyrrolidin-2-yl)methyl)carbamate (0.7 g, 47% yield for two steps) as a white solid. m/z found: 540 [M]⁺, 542 [M+2]⁺.

### tert-Butyl N-[[8-[3-[3-[5-[[tert-butoxycarbonyl-[[(2S)-5-oxopyrrolidin-2-yl]methyl]amino]methyl]-6-methoxy-pyrazin-2-yl]-2-chloro-phenyl]-2-methyl-phenyl]-4-oxo-quinolizin-3-yl]methyl]-N-[[(2S)-5-oxopyrrolidin-2-yl]methyl]carbamate

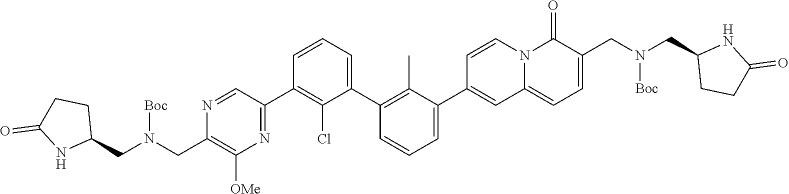 [SMILES: COc1nc(-c2cccc(-c3cccc(-c4ccn5c(=O)c(CN(C[C@@H]6CCC(=O)N6)C(=O)OC(C)(C)C)ccc5c4)c3C)c2Cl)cnc1CN(C[C@@H]1CCC(=O)N1)C(=O)OC(C)(C)C]

To a mixture of (S)-tert-butyl ((8-(3-bromo-2-methylphenyl)-4-oxo-4H-quinolizin-3-yl)methyl)((5-oxopyrrolidin-2-yl)methyl)carbamate (0.3 g, 0.55 mmol) and (S)-tert-butyl ((5-(2-chloro-3-(4,4,5,5-tetramethyl-1,3,2-dioxaborolan-2-yl)phenyl)-3-methoxypyrazin-2-yl)methyl)((5-oxopyrrolidin-2-yl)methyl)carbamate (0.32 g, 0.55 mmol) in dioxane (4.5 mL) and water (1.6 mL) was added 1,1′-bis(diphenylphosphino)ferrocene-palladium(II)dichloride dichloromethane complex (45 mg, 0.055 mmol) and potassium carbonate (0.23 g, 1.67 mmol). The mixture was stirred at 80° C. for 1 h. The reaction mixture was treated with water (10 mL), and extracted with EtOAc (2×10 mL). The combined organic layers were concentrated under reduced pressure. The residue was purified by normal phase SiO₂ chromatography (20-100% EtOAc/petroleum ether) to afford tert-butyl N-[[8-[3-[3-[5-[[tert-butoxycarbonyl-[[(2S)-5-oxopyrrolidin-2-yl]methyl]amino]methyl]-6-methoxy-pyrazin-2-yl]-2-chloro-phenyl]-2-methyl-phenyl]-4-oxo-quinolizin-3-yl]methyl]-N-[[(2S)-5-oxopyrrolidin-2-yl]methyl]carbamate (0.4 g, 79.50% yield) as a yellow solid. m/z found: 906 [M]⁺.

### 8-(2′-Chloro-3′-(6-methoxy-5-(((((S)-5-oxopyrrolidin-2-yl)methyl)amino)methyl)pyrazin-2-yl)-2-methyl-[1,1′-biphenyl]-3-yl)-3-(((((S)-5-oxopyrrolidin-2-yl)methyl)amino)methyl)-4H-quinolizin-4-one

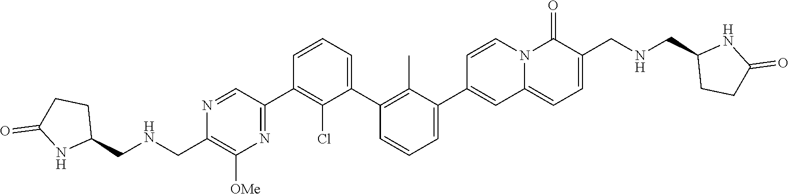 [SMILES: COc1nc(-c2cccc(-c3cccc(-c4ccn5c(=O)c(CNC[C@@H]6CCC(=O)N6)ccc5c4)c3C)c2Cl)cnc1CNC[C@@H]1CCC(=O)N1]

To a mixture of tert-butyl N-[[8-[3-[3-[5-[[tert-butoxycarbonyl-[[(2S)-5-oxopyrrolidin-2-yl]methyl]amino]methyl]-6-methoxy-pyrazin-2-yl]-2-chloro-phenyl]-2-methyl-phenyl]-4-oxo-quinolizin-3-yl]methyl]-N-[[(2S)-5-oxopyrrolidin-2-yl]methyl]carbamate (0.2 g, 0.2 mmol) in DCM (20 mL) was added TFA (4 mL, 54 mmol). The mixture was stirred at 25° C. for 12 h. The reaction mixture was filtered and concentrated under reduced pressure. The residue was purified by reverse phase HPLC to afford 8-(2′-chloro-3′-(6-methoxy-5-(((((S)-5-oxopyrrolidin-2-yl)methyl)amino)methyl)pyrazin-2-yl)-2-methyl-[1,1′-biphenyl]-3-yl)-3-(((((S)-5-oxopyrrolidin-2-yl)methyl)amino)methyl)-4H-quinolizin-4-one (29.16 mg, 18% yield) as a yellow solid. m/z found: 728 [M+Na]⁺. ¹H NMR (400 MHz, Methanol-d₄): δ 9.15 (d, J=7.6 Hz, 1H), 8.43 (s, 1H), 7.91 (d, J=7.6 Hz, 1H), 7.73 (s, 1H), 7.68 (m, 1H), 7.56 (t, J=7.6 Hz, 1H), 7.43 (m, 3H), 7.30 (m, 2H), 7.02 (d, J=8 Hz, 1H), 4.08 (s, 3H), 4.02 (s, 2H), 3.90 (m, 4H), 2.76 (m, 4H), 2.34 (m, 6H), 2.13 (s, 3H), 1.83 (m, 2H).

### Example 50: 8-(2′-Chloro-3′-(6-chloro-5-(((((S)-5-oxopyrrolidin-2-yl)methyl)amino)methyl)pyridin-2-yl)-2-methyl-[1,1′-biphenyl]-3-yl)-3-(((((S)-5-oxopyrrolidin-2-yl)methyl)amino)methyl)-4H-pyrido[1,2-a]pyrimidin-4-one

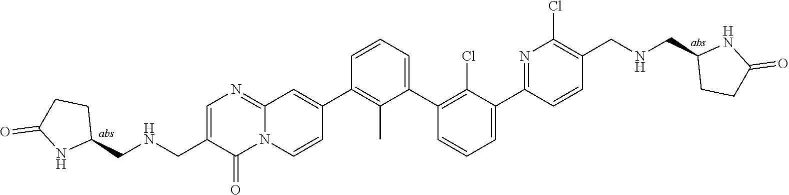 [SMILES: Cc1c(-c2ccn3c(=O)c(CNC[C@@H]4CCC(=O)N4)cnc3c2)cccc1-c1cccc(-c2ccc(CNC[C@@H]3CCC(=O)N3)c(Cl)n2)c1Cl]

### 8-[3-(3-Bromo-2-chloro-phenyl)-2-methyl-phenyl]-4-oxo-pyrido[1,2-a]pyrimidine-3-carbaldehyde

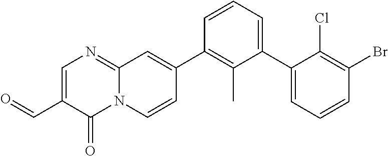 [SMILES: Cc1c(-c2ccn3c(=O)c(C=O)cnc3c2)cccc1-c1cccc(Br)c1Cl]

To a solution of 8-[2-methyl-3-(4,4,5,5-tetramethyl-1,3,2-dioxaborolan-2-yl)phenyl]-4-oxo-pyrido[1,2-a]pyrimidine-3-carbaldehyde (90 mg, 0.23 mmol), 1,3-dibromo-2-chloro-benzene (94 mg, 0.35 mmol) and potassium carbonate (96 mg, 0.69 mmol) in degassed dioxane/water (5:1) was added Pd(PPh₃)₄ (27 mg, 0.02 mmol) and the mixture was stirred at 95° C. for 2 hours under N₂(g). Water was added and the mixture was extracted into EtOAc three times. The combined organic layers were dried over anhydrous sodium sulfate, concentrated, and purified on silica gel column using EtOAc in hexanes (0 to 100% gradient) as eluent to give 8-[3-(3-bromo-2-chloro-phenyl)-2-methyl-phenyl]-4-oxo-pyrido[1,2-a]pyrimidine-3-carbaldehyde (58 mg, 55.4%). MS: m/z found 453.1 [M+H]⁺.

### 8-[3-[2-Chloro-3-(4,4,5,5-tetramethyl-1,3,2-dioxaborolan-2-yl)phenyl]-2-methyl-phenyl]-4-oxo-pyrido[1,2-a]pyrimidine-3-carbaldehyde

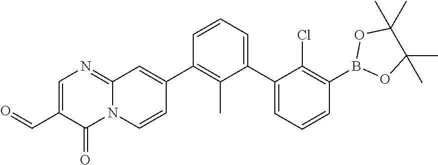 [SMILES: Cc1c(-c2ccn3c(=O)c(C=O)cnc3c2)cccc1-c1cccc(B2OC(C)(C)C(C)(C)O2)c1Cl]

To a solution of 8-[3-(3-bromo-2-chloro-phenyl)-2-methyl-phenyl]-4-oxo-pyrido[1,2-a]pyrimidine-3-carbaldehyde (50 mg, 0.11 mmol), 4,4,5,5-tetramethyl-2-(4,4,5,5-tetramethyl-1,3,2-dioxaborolan-2-yl)-1,3,2-dioxaborolane (84 mg, 0.33 mmol) and potassium acetate (32 mg, 0.33 mmol) in degassed dioxane (1 mL) under N₂ atmosphere was added Pd(dppf)Cl₂ (8.06 mg, 0.01 mmol). The reaction mixture was stirred at 85° C. for 12 hours, cooled to room temperature, diluted with water, and extracted with ethyl acetate. Organic layer was washed with brine, dried over sodium sulfate, and concentrated on a rotary evaporator. The crude residue was purified by flash column chromatography (ethyl acetate in hexanes, 0 to 100% gradient) to give 8-[3-[2-chloro-3-(4,4,5,5-tetramethyl-1,3,2-dioxaborolan-2-yl)phenyl]-2-methyl-phenyl]-4-oxo-pyrido[1,2-a]pyrimidine-3-carbaldehyde (35 mg, 63.4%). MS: m/z found 501.2 [M+H]⁺.

### (5S)-5-[[(6-bromo-2-chloro-3-pyridyl)methylamino]methyl]pyrrolidin-2-one

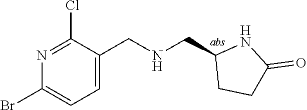 [SMILES: O=C1CC[C@@H](CNCc2ccc(Br)nc2Cl)N1]

A mixture of 6-bromo-2-chloro-pyridine-3-carbaldehyde (100 mg, 0.45 mmol), (5S)-5-(aminomethyl)pyrrolidin-2-one (104 mg, 0.91 mmol), acetic acid (26 uL, 27 mg, 0.45 mmol) and NaBH₃CN (57 mg, 0.91 mmol) were stirred in MeOH/THF (1:1) for 2 hours at rt. The mixture was concentrated, dissolved in EtOAc, and washed with water. The organic layer was dried over anhydrous sodium sulfate, concentrated, and used in the next step without purification. MS: m/z found 318.0 [M+H]⁺.

### tert-Butyl N-[(6-bromo-2-chloro-3-pyridyl)methyl]-N-[[(2S)-5-oxopyrrolidin-2-yl]methyl]carbamate

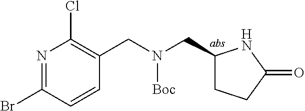 [SMILES: CC(C)(C)OC(=O)N(Cc1ccc(Br)nc1Cl)C[C@@H]1CCC(=O)N1]

A mixture of (5S)-5-[[(6-bromo-2-chloro-3-pyridyl)methylamino]methyl]pyrrolidin-2-one (130 mg, 0.41 mmol) and di-tert-butyl dicarbonate (134 mg, 0.61 mmol) in acetonitrile was stirred at rt overnight, concentrated and purified on silica gel column (MeOH in DCM, 0 to 10% gradient) to provide tert-butyl N-[(6-bromo-2-chloro-3-pyridyl)methyl]-N-[[(2S)-5-oxopyrrolidin-2-yl]methyl]carbamate (90 mg, 52.7%). MS: m/z found 418.1 [M+H]⁺.

### tert-Butyl N-[[2-chloro-6-[2-chloro-3-[3-(3-formyl-4-oxo-pyrido[1,2-a]pyrimidin-8-yl)-2-methyl-phenyl]phenyl]-3-pyridyl]methyl]-N-[[(2S)-5-oxopyrrolidin-2-yl]methyl]carbamate

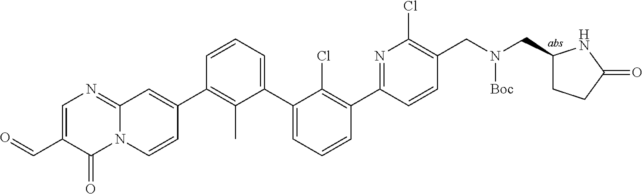 [SMILES: Cc1c(-c2ccn3c(=O)c(C=O)cnc3c2)cccc1-c1cccc(-c2ccc(CN(C[C@@H]3CCC(=O)N3)C(=O)OC(C)(C)C)c(Cl)n2)c1Cl]

To a solution of 8-[3-[2-chloro-3-(4,4,5,5-tetramethyl-1,3,2-dioxaborolan-2-yl)phenyl]-2-methyl-phenyl]-4-oxo-pyrido[1,2-a]pyrimidine-3-carbaldehyde (30 mg, 0.06 mmol), tert-butyl N-[(6-bromo-2-chloro-3-pyridyl)methyl]-N-[[(2S)-5-oxopyrrolidin-2-yl]methyl]carbamate (38 mg, 0.09 mmol) and potassium carbonate (25 mg, 0.18 mmol) in degassed dioxane/water (5:1) was added Pd(PPh₃)₄ (7 mg, 0.01 mmol) and the mixture was stirred at 90° C. overnight under N₂(g). Water was added and the mixture was extracted into EtOAc three times. The combined organic layers were dried over anhydrous sodium sulfate, concentrated, and purified by HPLC to give tert-butyl N-[[2-chloro-6-[2-chloro-3-[3-(3-formyl-4-oxo-pyrido[1,2-a]pyrimidin-8-yl)-2-methyl-phenyl]phenyl]-3-pyridyl]methyl]-N-[[(2S)-5-oxopyrrolidin-2-yl]methyl]carbamate (20 mg, 46.8%). MS: m/z found 712.3 [M+H]⁺.

### tert-Butyl N-[[2-chloro-6-[2-chloro-3-[2-methyl-3-[4-oxo-3-[[[(2S)-5-oxopyrrolidin-2-yl]methylamino]methyl]pyrido[1,2-a]pyrimidin-8-yl]phenyl]phenyl]-3-pyridyl]methyl]-N-[[(2S)-5-oxopyrrolidin-2-yl]methyl]carbamate

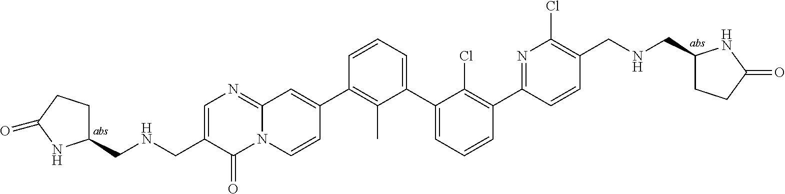 [SMILES: Cc1c(-c2ccn3c(=O)c(CNC[C@@H]4CCC(=O)N4)cnc3c2)cccc1-c1cccc(-c2ccc(CNC[C@@H]3CCC(=O)N3)c(Cl)n2)c1Cl]

A mixture of tert-butyl N-[[2-chloro-6-[2-chloro-3-[3-(3-formyl-4-oxo-pyrido[1,2-a]pyrimidin-8-yl)-2-methyl-phenyl]phenyl]-3-pyridyl]methyl]-N-[[(2S)-5-oxopyrrolidin-2-yl]methyl]carbamate (20 mg, 0.03 mmol), (5S)-5-(aminomethyl)pyrrolidin-2-one (6 mg, 0.06 mmol) and acetic acid (2 uL, 2 mg, 0.03 mmol) in MeOH/THF (1:1) was stirred in an iced-water bath for 1.5 hours. NaBH₃CN (4 mg, 0.06 mmol) was added, the mixture was quenched with water after 2 hours further stirring, and directly purified by HPLC to give tert-butyl N-[[2-chloro-6-[2-chloro-3-[2-methyl-3-[4-oxo-3-[[[(2S)-5-oxopyrrolidin-2-yl]methylamino]methyl]pyrido[1,2-a]pyrimidin-8-yl]phenyl]phenyl]-3-pyridyl]methyl]-N-[[(2S)-5-oxopyrrolidin-2-yl]methyl]carbamate (12 mg, 52.7%). MS: m/z found 810.4 [M+H]⁺.

### 8-[3-[2-Chloro-3-[6-chloro-5-[[[(2S)-5-oxopyrrolidin-2-yl]methylamino]methyl]-2-pyridyl]phenyl]-2-methyl-phenyl]-3-[[[(2S)-5-oxopyrrolidin-2-yl]methylamino]methyl]pyrido[1,2-a]pyrimidin-4-one

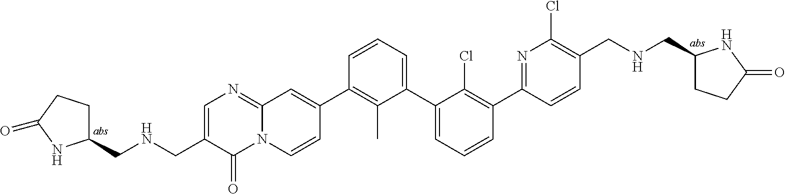 [SMILES: Cc1c(-c2ccn3c(=O)c(CNC[C@@H]4CCC(=O)N4)cnc3c2)cccc1-c1cccc(-c2ccc(CNC[C@@H]3CCC(=O)N3)c(Cl)n2)c1Cl]

tert-Butyl N-[[2-chloro-6-[2-chloro-3-[2-methyl-3-[4-oxo-3-[[[(2S)-5-oxopyrrolidin-2-yl]methylamino]methyl]pyrido[1,2-a]pyrimidin-8-yl]phenyl]phenyl]-3-pyridyl]methyl]-N-[[(2S)-5-oxopyrrolidin-2-yl]methyl]carbamate (12 mg, 0.01 mmol) in DCM—4M HCl in dioxane (5:1) (5 eq. HCl was used) was stirred for 45 minutes. The precipitated HCl salt of the product was filtered, washed with DCM three times and dried under vacuum. 8-[3-[2-chloro-3-[6-chloro-5-[[[(2S)-5-oxopyrrolidin-2-yl]methylamino]methyl]-2-pyridyl]phenyl]-2-methyl-phenyl]-3-[[[(2S)-5-oxopyrrolidin-2-yl]methylamino]methyl]pyrido[1,2-a]pyrimidin-4-one (10 mg, 95.1%) was obtained. MS: m/z found 710.3 [M+H]⁺. ¹H NMR (400 MHz, Methanol-d₄): δ 9.26 (dd, J=7.3, 0.6 Hz, 1H), 8.62 (s, 1H), 8.24 (d, J=7.9 Hz, 1H), 7.88-7.78 (m, 2H), 7.70 (dd, J=7.3, 1.9 Hz, 1H), 7.65-7.56 (m, 2H), 7.52-7.46 (m, 3H), 7.38 (dd, J=5.7, 3.3 Hz, 1H), 4.55 (d, J=1.8 Hz, 2H), 4.44-4.34 (m, 2H), 4.18-4.04 (m, 2H), 3.44-3.34 (m, 2H), 3.34-3.22 (m, 2H), 2.50-2.34 (m, 6H), 2.16 (s, 3H), 1.95 (dddd, J=16.7, 8.0, 6.8, 3.9 Hz, 2H).

### Example 51: (5S)-5-(((7-(2,2′-Dichloro-3′-(6-methoxy-5-(((((S)-5-oxopyrrolidin-2-yl)methyl)amino)methyl)pyridin-2-yl)-[1,1′-biphenyl]-3-yl)-1-methyl-1,2,3,4-tetrahydro-1,8-naphthyridin-4-yl)amino)methyl)pyrrolidin-2-one

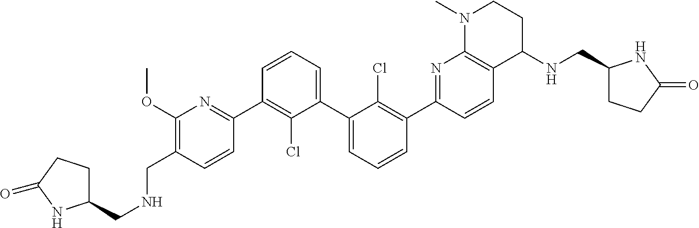 [SMILES: COc1nc(-c2cccc(-c3cccc(-c4ccc5c(n4)N(C)CCC5NC[C@@H]4CCC(=O)N4)c3Cl)c2Cl)ccc1CNC[C@@H]1CCC(=O)N1]

### tert-Butyl ((6-(3′-(5-((tert-butoxycarbonyl)(((S)-5-oxopyrrolidin-2-yl)methyl)amino)-8-methyl-5,6,7,8-tetrahydro-1,8-naphthyridin-2-yl)-2,2′-dichloro-[1,1′-biphenyl]-3-yl)-2-methoxypyridin-3-yl)methyl)(((S)-5-oxopyrrolidin-2-yl)methyl)carbamate

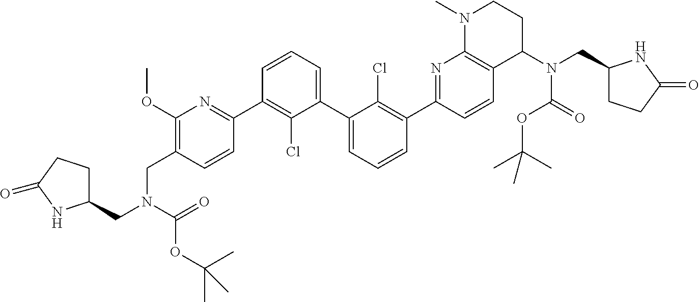 [SMILES: COc1nc(-c2cccc(-c3cccc(-c4ccc5c(n4)N(C)CCC5N(C[C@@H]4CCC(=O)N4)C(=O)OC(C)(C)C)c3Cl)c2Cl)ccc1CN(C[C@@H]1CCC(=O)N1)C(=O)OC(C)(C)C]

Pd(PPh₃)₄ (15 mg, 0.01 mmol), potassium carbonate (26 mg, 0.19 mmol), 2-[2-chloro-3-[2-chloro-3-(4,4,5,5-tetramethyl-1,3,2-dioxaborolan-2-yl)phenyl]phenyl]-4,4,5,5-tetramethyl-1,3,2-dioxaborolane (30 mg, 0.06 mmol), tert-butyl N-[(6-chloro-2-methoxy-3-pyridyl)methyl]-N-[[(2S)-5-oxopyrrolidin-2-yl]methyl]carbamate (23 mg, 0.06 mmol) and tert-butyl N-(7-bromo-1-methyl-3,4-dihydro-2H-1,8-naphthyridin-4-yl)-N-[[(2S)-5-oxopyrrolidin-2-yl]methyl]carbamate (28 mg, 0.06 mmol), were suspended in 1 ml of 4:1 dioxane/water solvent mixture, and the solution was heated at 105° C. for 1 hour. The reaction was cooled to rt, filtered through CELITE®, and the filtrate was concentrated. Crude product was purified by reverse phase HPLC to afford tert-butyl N-[[6-[3-[3-[5-[tert-butoxycarbonyl-[[(2S)-5-oxopyrrolidin-2-yl]methyl]amino]-8-methyl-6,7-dihydro-5H-1,8-naphthyridin-2-yl]-2-chloro-phenyl]-2-chloro-phenyl]-2-methoxy-3-pyridyl]methyl]-N-[[(2S)-5-oxopyrrolidin-2-yl]methyl]carbamate (19 mg, 32.9% yield) as a clear film (racemic mixture of diastereomers). MS: m/z found 914.5, 916.5 [M+H]⁺.

### (5S)-5-(((7-(2,2′-Dichloro-3′-(6-methoxy-5-(((((S)-5-oxopyrrolidin-2-yl)methyl)amino)methyl)pyridin-2-yl)-[1,1′-biphenyl]-3-yl)-1-methyl-1,2,3,4-tetrahydro-1,8-naphthyridin-4-yl)amino)methyl)pyrrolidin-2-one

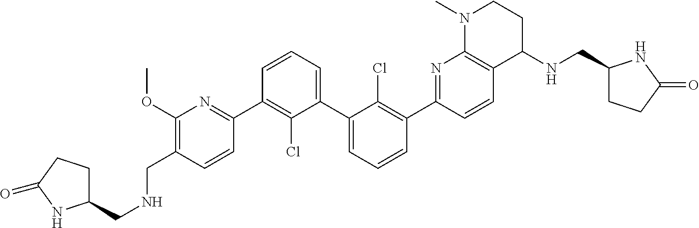 [SMILES: COc1nc(-c2cccc(-c3cccc(-c4ccc5c(n4)N(C)CCC5NC[C@@H]4CCC(=O)N4)c3Cl)c2Cl)ccc1CNC[C@@H]1CCC(=O)N1]

tert-Butyl-rac-N-[[6-[3-[3-[5-[tert-butoxycarbonyl-[[(2S)-5-oxopyrrolidin-2-yl]methyl]amino]-8-methyl-6,7-dihydro-5H-1,8-naphthyridin-2-yl]-2-chloro-phenyl]-2-chloro-phenyl]-2-methoxy-3-pyridyl]methyl]-N-[[(2S)-5-oxopyrrolidin-2-yl]methyl]carbamate (19 mg, 0.02 mmol) was dissolved in 0.5 ml DCM and 4M HCl solution in dioxane (26 μL, 0.10 mmol) was added. The reaction mixture was stirred at rt for 20 minutes, then the sample was concentrated under vacuum. The crude product was purified by reverse phase HPLC to afford (5S)-5-[[[6-[2-chloro-3-[2-chloro-3-[8-methyl-5-[[(2S)-5-oxopyrrolidin-2-yl]methylamino]-6,7-dihydro-5H-1,8-naphthyridin-2-yl]phenyl]phenyl]-2-methoxy-3-pyridyl]methylamino]methyl]pyrrolidin-2-one (6 mg, 39% yield) as a clear film (racemic mixture of diastereomers). MS: m/z found 715, 717 [M+H]⁺. ¹H NMR (400 MHz, Chloroform-d) δ 7.69-7.60 (m, 2H), 7.57 (d, J=7.4 Hz, 1H), 7.41 (q, J=7.9 Hz, 3H), 7.31 (ddd, J=16.7, 5.7, 2.9 Hz, 3H), 6.92-6.84 (m, 1H), 6.02 (s, 2H), 4.03 (s, 3H), 3.84-3.67 (m, 5H), 3.50 (d, J=10.7 Hz, 1H), 3.31 (t, J=12.4 Hz, 1H), 3.19 (d, J=2.7 Hz, 3H), 2.93 (d, J=11.2 Hz, 1H), 2.79 (dd, J=12.0, 4.2 Hz, 1H), 2.66-2.49 (m, 2H), 2.37 (ddd, J=9.9, 7.0, 2.4 Hz, 4H), 2.24 (dq, J=14.7, 7.3 Hz, 2H), 2.09-1.86 (m, 2H), 1.77 (td, J=14.9, 8.1 Hz, 2H).

### Example 52 and Example 53: (S)-5-((((6-(2,2′-Dichloro-3′-((S)-8-methyl-5-((((S)-5-oxopyrrolidin-2-yl)methyl)amino)-5,6,7,8-tetrahydro-1,8-naphthyridin-2-yl)-[1,1′-biphenyl]-3-yl)-2-methoxypyridin-3-yl)methyl)amino)methyl)pyrrolidin-2-one isomer

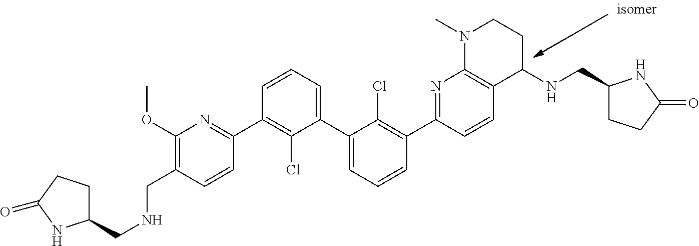 [SMILES: COc1nc(-c2cccc(-c3cccc(-c4ccc5c(n4)N(C)CCC5NC[C@@H]4CCC(=O)N4)c3Cl)c2Cl)ccc1CNC[C@@H]1CCC(=O)N1]

### Separation of diastereomers for (5S)-5-(((7-bromo-1-methyl-1,2,3,4-tetrahydro-1,8-naphthyridin-4-yl)-rac-amino)-methyl)-pyrrolidin-2-one

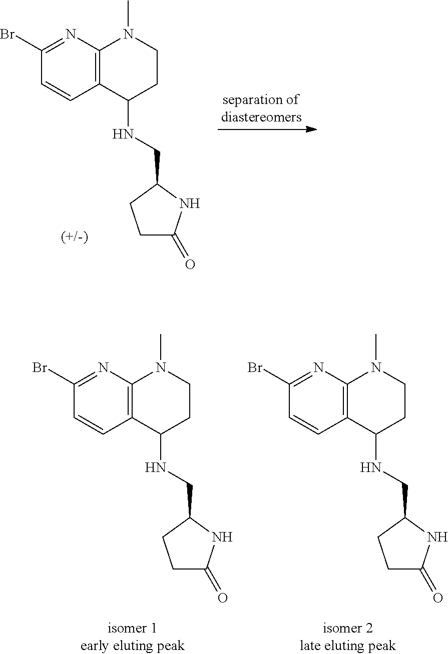 [SMILES: CN1CCC(NC[C@@H]2CCC(=O)N2)c2ccc(Br)nc21 ; CN1CCC(NC[C@@H]2CCC(=O)N2)c2ccc(Br)nc21 ; CN1CCC(NC[C@@H]2CCC(=O)N2)c2ccc(Br)nc21]

100 mg of diastereomeric (5S)-5-[[(7-bromo-1-methyl-3,4-dihydro-2H-1,8-naphthyridin-4-yl) amino]methyl]-pyrrolidin-2-one was subjected to reverse phase HPLC separation, eluting with 3-40% acetonitrile gradient in water with 0.1% TFA, to obtain the two single diastereomers.

Fractions of the early eluting peak were combined and lyophilized to afford isomer 1 (5S)-5-[[(7-bromo-1-methyl-3,4-dihydro-2H-1,8-naphthyridin-4-yl)-amino]methyl]-pyrrolidin-2-one (17.6 mg) as white foam. MS: m/z found 339, 341 [M+H]⁺. ¹H NMR (400 MHz, Methanol-d₄) δ 7.37 (d, J=7.7 Hz, 1H), 6.73 (d, J=7.6 Hz, 1H), 4.46 (t, J=3.5 Hz, 1H), 4.00 (q, J=6.7 Hz, 1H), 3.54-3.44 (m, 2H), 3.14 (s, 4H), 2.44-2.30 (m, 4H), 2.27-2.10 (m, 1H), 1.99-1.82 (m, 1H), 1.37 (dd, J=6.7, 3.6 Hz, 1H).

Fractions of the later eluting peak were combined and lyophilized to afford isomer 2 (5S)-5-[[(7-bromo-1-methyl-3,4-dihydro-2H-1,8-naphthyridin-4-yl)-amino]methyl]-pyrrolidin-2-one (14.5 mg) as white foam. MS: m/z found 339, 341 [M+H]⁺. ¹H NMR (400 MHz, Methanol-d₄) δ 7.37 (dd, J=7.8, 1.3 Hz, 1H), 6.73 (dd, J=7.6, 1.1 Hz, 1H), 4.45 (t, J=3.6 Hz, 1H), 4.04-3.90 (m, 1H), 3.55-3.42 (m, 2H), 3.27-3.16 (m, 2H), 3.14 (d, J=1.1 Hz, 3H), 2.46-2.31 (m, 4H), 2.26-2.11 (m, 1H), 1.89 (ddt, J=11.7, 9.8, 5.6 Hz, 1H).

Single Diastereomers were Used to Synthesize Example 52 and 53 as Described in Example 51.

### Example 52: (5S)-5-(((7-(2,2′-Dichloro-3′-(6-methoxy-5-(((((S)-5-oxopyrrolidin-2-yl)methyl)amino)methyl)pyridin-2-yl)-[1,1′-biphenyl]-3-yl)-1-methyl-1,2,3,4-tetrahydro-1,8-naphthyridin-4-yl)amino)methyl)pyrrolidin-2-one

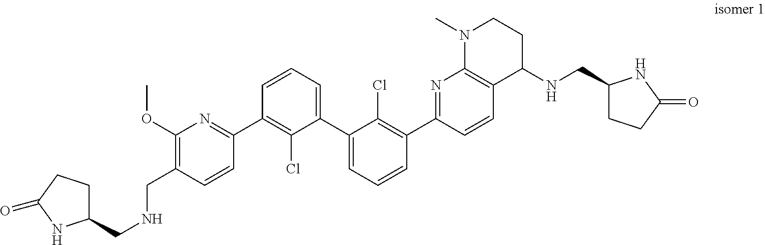 [SMILES: COc1nc(-c2cccc(-c3cccc(-c4ccc5c(n4)N(C)CCC5NC[C@@H]4CCC(=O)N4)c3Cl)c2Cl)ccc1CNC[C@@H]1CCC(=O)N1]

MS: m/z found 715, 717 [M+H]⁺. ¹H NMR (400 MHz, Chloroform-d) δ 7.66 (ddd, J=7.1, 5.2, 1.7 Hz, 2H), 7.59 (d, J=7.5 Hz, 1H), 7.44-7.36 (m, 3H), 7.32 (dt, J=7.6, 1.8 Hz, 1H), 7.29 (dt, J=7.4, 1.5 Hz, 2H), 6.89 (dd, J=7.4, 3.1 Hz, 1H), 6.16 (s, 1H), 6.10 (s, 1H), 4.03 (s, 3H), 3.83-3.71 (m, 4H), 3.49 (s, 2H), 3.36-3.28 (m, 1H), 3.18 (s, 3H), 2.93 (ddd, J=11.8, 4.5, 2.7 Hz, 1H), 2.81 (dd, J=12.1, 4.1 Hz, 1H), 2.66-2.53 (m, 2H), 2.36 (dd, J=8.9, 7.4 Hz, 4H), 2.31-2.17 (m, 2H), 2.03 (d, J=19.1 Hz, 1H), 1.90 (dd, J=9.3, 4.5 Hz, 1H), 1.84-1.69 (m, 6H).

### Example 53: (5S)-5-(((7-(2,2′-Dichloro-3′-(6-methoxy-5-(((((S)-5-oxopyrrolidin-2-yl)methyl)amino)methyl)pyridin-2-yl)-[1,1′-biphenyl]-3-yl)-1-methyl-1,2,3,4-tetrahydro-1,8-naphthyridin-4-yl)amino)methyl)pyrrolidin-2-one

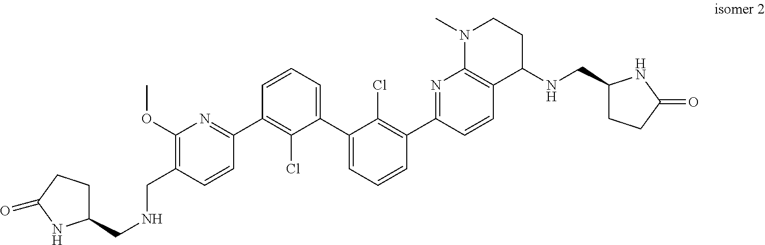 [SMILES: COc1nc(-c2cccc(-c3cccc(-c4ccc5c(n4)N(C)CCC5NC[C@@H]4CCC(=O)N4)c3Cl)c2Cl)ccc1CNC[C@@H]1CCC(=O)N1]

MS: m/z found 715, 717 [M+H]⁺. ¹H NMR (400 MHz, Chloroform-d) δ 7.66 (ddd, J=8.0, 4.3, 1.7 Hz, 2H), 7.57 (d, J=7.5 Hz, 1H), 7.40 (q, J=8.0 Hz, 2H), 7.34-7.27 (m, 4H), 6.88 (dd, J=7.4, 2.7 Hz, 1H), 6.08 (s, 2H), 4.02 (d, J=1.4 Hz, 3H), 3.84-3.66 (m, 5H), 3.52 (t, J=13.5 Hz, 1H), 3.34-3.27 (m, 1H), 3.19 (d, J=1.4 Hz, 3H), 2.93 (d, J=11.8 Hz, 1H), 2.79 (dd, J=12.0, 4.2 Hz, 1H), 2.59 (ddd, J=21.0, 11.8, 8.7 Hz, 2H), 2.36 (td, J=7.6, 2.2 Hz, 4H), 2.30-2.20 (m, 2H), 1.94 (d, J=5.2 Hz, 2H), 1.85-1.70 (m, 2H).

### Example 54: 8-(2′-Chloro-3′-(6-methoxy-5-(((((S)-5-oxopyrrolidin-2-yl)methyl)amino)methyl)pyridin-2-yl)-2-methyl-[1,1′-biphenyl]-3-yl)-3-((methyl(((S)-5-oxopyrrolidin-2-yl)methyl)amino)methyl)-4H-pyrido[1,2-a]pyrimidin-4-one

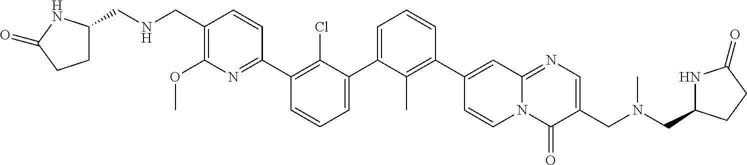 [SMILES: COc1nc(-c2cccc(-c3cccc(-c4ccn5c(=O)c(CN(C)C[C@@H]6CCC(=O)N6)cnc5c4)c3C)c2Cl)ccc1CNC[C@@H]1CCC(=O)N1]

### (S)-8-(3-Bromo-2-methylphenyl)-3-((((5-oxopyrrolidin-2-yl)-methyl)-amino)-methyl)-4H-pyrido[1,2-a]-pyrimidin-4-one

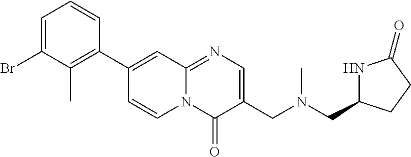 [SMILES: Cc1c(Br)cccc1-c1ccn2c(=O)c(CN(C)C[C@@H]3CCC(=O)N3)cnc2c1]

8-(3-Bromo-2-methyl-phenyl)-4-oxo-pyrido[1,2-a]pyrimidine-3-carbaldehyde (150 mg, 0.44 mmol), (5S)-5-(aminomethyl)-pyrrolidin-2-one (65 mg, 0.57 mmol), and acetic acid (32 mg, 0.52 mmol) were dissolved in 2 ml of 1:1 MeOH/THF solvent mix, and the reaction was stirred at 25° C. for 3 hours. NaBH₃CN (41 mg, 0.66 mmol) was then added portion-wise and the resulting mixture was stirred at rt for 1 hour. Reaction mixture was diluted with water (10 ml) and extracted with EtOAc (3×5 ml). The combined organics were further washed with brine, dried over sodium sulfate, then filtered and concentrated to afford 8-(3-bromo-2-methyl-phenyl)-3-[[[(2S)-5-oxopyrrolidin-2-yl]methylamino]methyl]pyrido[1,2-a]pyrimidin-4-one (195 mg) as a light yellow solid. Material was used as—is for the next step. MS: m/z found 442, 444 [M+H]^{m}.

### (S)-8-(3-Bromo-2-methylphenyl)-3-((methyl((5-oxopyrrolidin-2-yl)-methyl)-amino)-methyl)-4H-pyrido[1,2-a]-pyrimidin-4-one

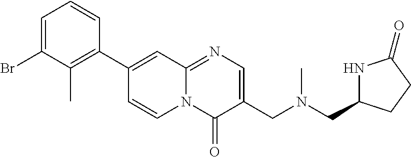 [SMILES: Cc1c(Br)cccc1-c1ccn2c(=O)c(CN(C)C[C@@H]3CCC(=O)N3)cnc2c1]

8-(3-Bromo-2-methyl-phenyl)-3-[[[(2S)-5-oxopyrrolidin-2-yl]methylamino]methyl]pyrido[1,2-a]pyrimidin-4-one (80 mg, 0.18 mmol), formaldehyde (91 μL, 90 mg, 0.91 mmol), sodium triacetoxyborohydride (77 mg, 0.36 mmol) and acetic acid (15 mg, 0.25 mmol) were dissolved in 2 ml THE and the mixture was stirred at 25° C. for 12 hours. The reaction was subsequently diluted with water (5 ml) and extracted with EtOAc (3×5 ml). The combined organics were concentrated and the crude residue was purified by silica gel chromatography, eluting with 0-10% MeOH gradient in DCM to afford 8-(3-bromo-2-methyl-phenyl)-3-[[methyl-[[(2S)-5-oxopyrrolidin-2-yl]methyl]amino]methyl]pyrido[1,2-a]pyrimidin-4-one (33 mg, 40% yield) as a white solid. MS: m/z found 455, 457.1 [M+H]⁺.

### (S)-6-(2-Chloro-2′-methyl-3′-(3-((methyl((5-oxopyrrolidin-2-yl)methyl)amino)methyl)-4-oxo-4H-pyrido[1,2-a]pyrimidin-8-yl)-[1,1′-biphenyl]-3-yl)-2-methoxynicotinaldehyde

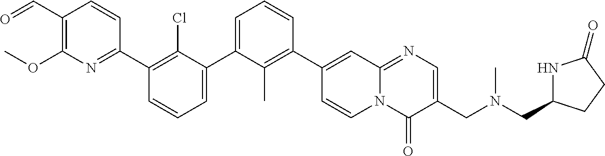 [SMILES: COc1nc(-c2cccc(-c3cccc(-c4ccn5c(=O)c(CN(C)C[C@@H]6CCC(=O)N6)cnc5c4)c3C)c2Cl)ccc1C=O]

8-(3-Bromo-2-methyl-phenyl)-3-[[methyl-[[(2S)-5-oxopyrrolidin-2-yl]methyl]amino]methyl]pyrido[1,2-a]pyrimidin-4-one (33 mg, 0.07 mmol), 6-[2-chloro-3-(4,4,5,5-tetramethyl-1,3,2-dioxaborolan-2-yl)phenyl]-2-methoxy-pyridine-3-carbaldehyde (31 mg, 0.08 mmol), 1,1′-bis(diphenylphosphino)-ferrocene-palladium(ii) dichloride dichloromethane complex (6 mg, 0.01 mmol) and potassium carbonate (25 mg, 0.18 mmol) were suspended in 1 ml of 9:1 dioxane/water solvent mix in a sealed tube. Nitrogen gas was bubbled through the reaction solution for 2 minutes, then the vessel was sealed and heated at 95° C. for 2 hours. The reaction was cooled to rt and the mixture was diluted with 5 ml water and extracted with EtOAc (3×2 ml). The combined organics were concentrated and the crude oil was purified by silica gel chromatography, eluting with 0-7% MeOH gradient in DCM to afford 6-[2-chloro-3-[2-methyl-3-[3-[[methyl-[[(2S)-5-oxopyrrolidin-2-yl]methyl]amino]methyl]-4-oxo-pyrido[1,2-a]pyrimidin-8-yl]phenyl]phenyl]-2-methoxy-pyridine-3-carbaldehyde (32 mg, 69.9% yield) as a light brown oil. MS: m/z found 622.3, 624.25 [M+H]⁺.

### 8-(2′-Chloro-3′-(6-methoxy-5-(((((S)-5-oxopyrrolidin-2-yl)methyl)amino)methyl)pyridin-2-yl)-2-methyl-[1,1′-biphenyl]-3-yl)-3-((methyl(((S)-5-oxopyrrolidin-2-yl)methyl)amino)methyl)-4H-pyrido[1,2-a]pyrimidin-4-one

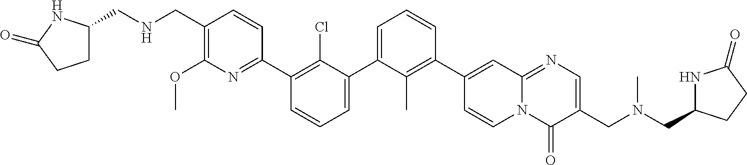 [SMILES: COc1nc(-c2cccc(-c3cccc(-c4ccn5c(=O)c(CN(C)C[C@@H]6CCC(=O)N6)cnc5c4)c3C)c2Cl)ccc1CNC[C@@H]1CCC(=O)N1]

6-[2-Chloro-3-[2-methyl-3-[3-[[methyl-[[(2S)-5-oxopyrrolidin-2-yl]methyl]amino]methyl]-4-oxo-pyrido[1,2-a]pyrimidin-8-yl]phenyl]phenyl]-2-methoxy-pyridine-3-carbaldehyde (15 mg, 0.02 mmol), (5S)-5-(aminomethyl)pyrrolidin-2-one (4 mg, 0.04 mmol) and acetic acid (2 mg, 0.03 mmol) were dissolved in 1 ml of 1:1 MeOH/THF solvent mix, and the reaction was stirred at 25° C. for 3 hours. NaBH₃CN, (2 mg, 0.04 mmol) was then added portion-wise and the resulting mixture was stirred at rt for 1 hour. The reaction was subsequently diluted with water (5 ml) and extracted with EtOAc (3×2 ml). The combined organics were further washed with brine, dried over sodium sulfate, then filtered and concentrated. The crude product was purified by reverse phase HPLC to afford 8-[3-[2-chloro-3-[6-methoxy-5-[[[(2S)-5-oxopyrrolidin-2-yl]methylamino]methyl]-2-pyridyl]phenyl]-2-methyl-phenyl]-3-[[methyl-[[(2S)-5-oxopyrrolidin-2-yl]methyl]amino]methyl]pyrido[1,2-a]pyrimidin-4-one (6.3 mg, 36.3%) yield as a clear film. MS: m/z found 720.4, 722.4 [M+H]⁺. ¹H NMR (400 MHz, Chloroform-d) δ 9.14 (d, J=7.3 Hz, 1H), 8.27 (s, 1H), 7.65 (dd, J=7.9, 1.7 Hz, 2H), 7.59 (d, J=7.5 Hz, 1H), 7.42 (dt, J=10.8, 7.5 Hz, 2H), 7.37-7.28 (m, 3H), 7.26-7.22 (m, 2H), 7.05 (s, 1H), 6.05 (s, 1H), 4.03 (s, 4H), 3.84-3.74 (m, 4H), 3.36 (d, J=13.2 Hz, 1H), 2.80 (dd, J=12.0, 4.2 Hz, 1H), 2.61-2.45 (m, 3H), 2.41-2.33 (m, 4H), 2.29 (s, 3H), 2.28-2.19 (m, 2H), 2.14 (s, 3H), 1.81-1.71 (m, 4H).

### Example 55: (S)-8-(2′-Chloro-3′-(5-((3-hydroxyazetidin-1-yl)methyl)-6-methoxypyridin-2-yl)-2-methyl-[1,1′-biphenyl]-3-yl)-3-((methyl((5-oxopyrrolidin-2-yl)methyl)amino)methyl)-4H-pyrido[1,2-a]pyrimidin-4-one

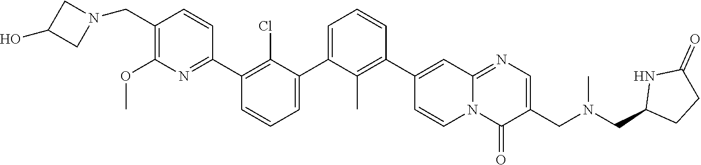 [SMILES: COc1nc(-c2cccc(-c3cccc(-c4ccn5c(=O)c(CN(C)C[C@@H]6CCC(=O)N6)cnc5c4)c3C)c2Cl)ccc1CN1CC(O)C1]

6-[2-Chloro-3-[2-methyl-3-[3-[[methyl-[[(2S)-5-oxopyrrolidin-2-yl]methyl]amino]methyl]-4-oxo-pyrido[1,2-a]pyrimidin-8-yl]phenyl]phenyl]-2-methoxy-pyridine-3-carbaldehyde (15 mg, 0.02 mmol), azetidin-3-ol 3 mg, 0.04 mmol) and acetic acid (2 mg, 0.03 mmol were dissolved in 1 ml of 1:1 MeOH/THF solvent mix, and the reaction was stirred at 25° C. for 3 hours. NaBH₃CN (2 mg, 0.04 mmol) was then added and the resulting mixture was stirred at rt for 1 hour. The reaction was subsequently diluted with water (3 ml) and extracted with EtOAc (3×2 ml). The combined organics were further washed with brine, dried over sodium sulfate, then filtered and concentrated. The crude product was purified by reverse phase HPLC to afford 8-[3-[2-chloro-3-[5-[(3-hydroxyazetidin-1-yl)methyl]-6-methoxy-2-pyridyl]phenyl]-2-methyl-phenyl]-3-[[methyl-[[(2S)-5-oxopyrrolidin-2-yl]methyl]amino]methyl]pyrido[1,2-a]pyrimidin-4-one (1.4 mg, 8.5%) as a clear oil. MS: m/z found 680, 682 [M+H]⁺. 1H NMR (400 MHz, Chloroform-d) δ 9.14 (d, J=7.4 Hz, 1H), 8.27 (s, 1H), 7.69-7.56 (m, 3H), 7.46-7.38 (m, 2H), 7.37-7.28 (m, 3H), 7.27 (s, 1H), 7.23 (d, J=1.9 Hz, 1H), 7.03 (s, 1H), 4.58-4.46 (m, 1H), 4.01 (d, J=2.8 Hz, 3H), 3.81 (s, 2H), 3.72 (s, 2H), 3.35 (d, J=13.2 Hz, 1H), 3.14 (s, 2H), 2.61-2.46 (m, 2H), 2.43-2.33 (m, 2H), 2.29 (s, 4H), 2.14 (s, 3H), 1.36 (d, J=50.3 Hz, 4H).

### Example 56: (5S)-5-(((7-(2,2′-Dichloro-3′-(6-methoxy-5-(((((S)-5-oxopyrrolidin-2-yl)methyl)amino)methyl)pyrazin-2-yl)-[1,1′-biphenyl]-3-yl)-1-methyl-1,2,3,4-tetrahydro-1,8-naphthyridin-4-yl)amino)methyl)pyrrolidin-2-one

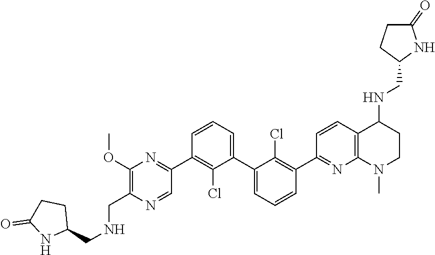 [SMILES: COc1nc(-c2cccc(-c3cccc(-c4ccc5c(n4)N(C)CCC5NC[C@@H]4CCC(=O)N4)c3Cl)c2Cl)cnc1CNC[C@@H]1CCC(=O)N1]

### tert-Butyl ((5-(3′-(5-((tert-butoxycarbonyl)(((S)-5-oxopyrrolidin-2-yl)methyl)amino)-8-methyl-5,6,7,8-tetrahydro-1,8-naphthyridin-2-yl)-2,2′-dichloro-[1,1′-biphenyl]-3-yl)-3-methoxypyrazin-2-yl)methyl)(((S)-5-oxopyrrolidin-2-yl)methyl)carbamate

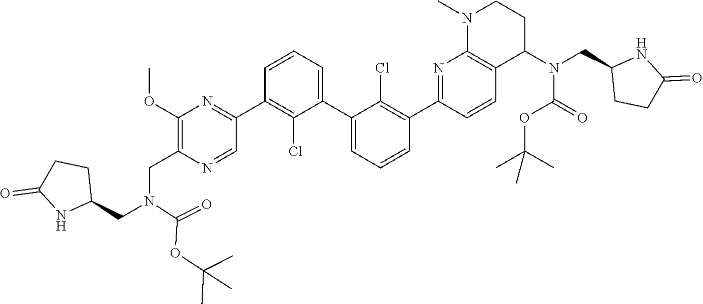 [SMILES: COc1nc(-c2cccc(-c3cccc(-c4ccc5c(n4)N(C)CCC5N(C[C@@H]4CCC(=O)N4)C(=O)OC(C)(C)C)c3Cl)c2Cl)cnc1CN(C[C@@H]1CCC(=O)N1)C(=O)OC(C)(C)C]

Pd(PPh₃)₄ (17 mg, 0.01 mmol), potassium carbonate, (31 mg, 0.22 mmol), 2-[2-chloro-3-[2-chloro-3-(4,4,5,5-tetramethyl-1,3,2-dioxaborolan-2-yl)phenyl]phenyl]-4,4,5,5-tetramethyl-1,3,2-dioxaborolane (35 mg, 0.07 mmol), tert-butyl N-(7-bromo-1-methyl-3,4-dihydro-2H-1,8-naphthyridin-4-yl)-N-[[(2S)-5-oxopyrrolidin-2-yl]methyl]carbamate (32 mg, 0.07 mmol), and tert-butyl N-[(5-bromo-3-methoxy-pyrazin-2-yl)methyl]-N-[[(2S)-5-oxopyrrolidin-2-yl]methyl]carbamate (31 mg, 0.07 mmol) were suspended in 1 ml of 4:1 dioxane/water solvent mixture, and the solution was heated at 105° C. for 1 hour. The reaction was cooled to rt, filtered through CELITE®, and the filtrate was concentrated. Crude sample was purified by reverse phase HPLC to afford tert-butyl N-[[5-[3-[3-[5-[tert-butoxycarbonyl-[[(2S)-5-oxopyrrolidin-2-yl]methyl]amino]-8-methyl-6,7-dihydro-5H-1,8-naphthyridin-2-yl]-2-chloro-phenyl]-2-chloro-phenyl]-3-methoxy-pyrazin-2-yl]methyl]-N-[[(2S)-5-oxopyrrolidin-2-yl]methyl]carbamate (31 mg, 45.9% yield) as a clear film (racemic mixture of diastereomers). MS: m/z found 915.5, 917.5 [M+H]⁺.

### (5S)-5-(((7-(2,2′-Dichloro-3′-(6-methoxy-5-(((((S)-5-oxopyrrolidin-2-yl)methyl)amino)methyl)pyrazin-2-yl)-[1,1′-biphenyl]-3-yl)-1-methyl-1,2,3,4-tetrahydro-1,8-naphthyridin-4-yl)amino)methyl)pyrrolidin-2-one

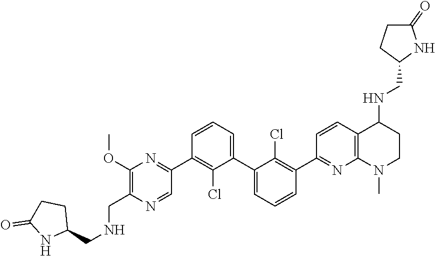 [SMILES: COc1nc(-c2cccc(-c3cccc(-c4ccc5c(n4)N(C)CCC5NC[C@@H]4CCC(=O)N4)c3Cl)c2Cl)cnc1CNC[C@@H]1CCC(=O)N1]

tert-Butyl N-[[5-[3-[3-[5-[tert-butoxycarbonyl-[[(2S)-5-oxopyrrolidin-2-yl]methyl]amino]-8-methyl-6,7-dihydro-5H-1,8-naphthyridin-2-yl]-2-chloro-phenyl]-2-chloro-phenyl]-3-methoxy-pyrazin-2-yl]methyl]-N-[[(2S)-5-oxopyrrolidin-2-yl]methyl]carbamate (31 mg, 0.03 mmol) was dissolved in 0.5 ml DCM and 4M HCl solution in dioxane (42 μL, 6 mg, 0.17 mmol) was added. The reaction was stirred at rt for 30 minutes and the precipitate was filtered and further washed with DCM (5 ml). The resulting solid was dissolved in 2 ml water and the pH was adjusted to ˜ pH 9 by slow addition of saturated aqueous sodium bicarbonate solution. The aqueous was extracted with DCM (3×1 ml) and the combined organics were washed with brine, dried over sodium sulfate, filtered and concentrated to afford (5S)-5-[[[5-[2-chloro-3-[2-chloro-3-[8-methyl-5-[[(2S)-5-oxopyrrolidin-2-yl]methylamino]-6,7-dihydro-5H-1,8-naphthyridin-2-yl]phenyl]phenyl]-3-methoxy-pyrazin-2-yl]methylamino]methyl]pyrrolidin-2-one (12 mg, 49.5% yield) as a clear oil (racemic mixture of diastereomers). MS: m/z found 715, 717 [M+H]⁺. ¹H NMR (400 MHz, Chloroform-d) δ 8.48 (s, 1H), 7.70-7.59 (m, 2H), 7.48-7.42 (m, 1H), 7.41-7.36 (m, 2H), 7.35-7.27 (m, 2H), 6.88 (dt, J=7.6, 2.6 Hz, 1H), 6.07 (s, 1H), 5.98 (d, J=7.4 Hz, 1H), 4.08-3.96 (m, 5H), 3.78 (d, J=26.6 Hz, 3H), 3.60-3.41 (m, 1H), 3.31 (t, J=13.0 Hz, 1H), 3.19 (d, J=2.7 Hz, 3H), 3.01-2.79 (m, 2H), 2.64 (dq, J=17.4, 9.4, 8.9 Hz, 2H), 2.37 (td, J=7.1, 2.8 Hz, 4H), 2.25 (q, J=9.0, 7.6 Hz, 2H), 2.00 (d, J=44.9 Hz, 2H), 1.79 (dq, J=15.4, 8.0 Hz, 2H).

### Example 57: 8-(2′-Chloro-3′-(6-fluoro-5-(((((S)-5-oxopyrrolidin-2-yl)methyl)amino)methyl)pyridin-2-yl)-2-methyl-[1,1′-biphenyl]-3-yl)-3-(((((S)-5-oxopyrrolidin-2-yl)methyl)amino)methyl)-4H-pyrido[1,2-a]pyrimidin-4-one

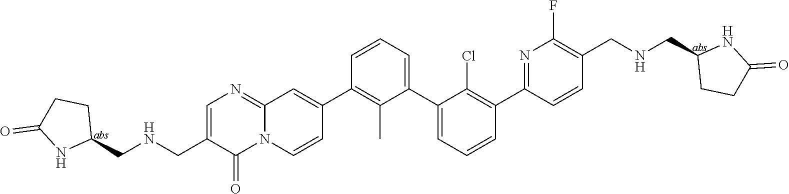 [SMILES: Cc1c(-c2ccn3c(=O)c(CNC[C@@H]4CCC(=O)N4)cnc3c2)cccc1-c1cccc(-c2ccc(CNC[C@@H]3CCC(=O)N3)c(F)n2)c1Cl]

### 8-[3-[2-Chloro-3-(6-fluoro-5-formyl-2-pyridyl)phenyl]-2-methyl-phenyl]-4-oxo-pyrido[1,2-a]pyrimidine-3-carbaldehyde

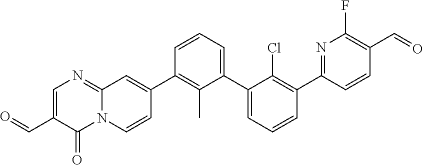 [SMILES: Cc1c(-c2ccn3c(=O)c(C=O)cnc3c2)cccc1-c1cccc(-c2ccc(C=O)c(F)n2)c1Cl]

To a solution of 8-[3-[2-chloro-3-(4,4,5,5-tetramethyl-1,3,2-dioxaborolan-2-yl)phenyl]-2-methyl-phenyl]-4-oxo-pyrido[1,2-a]pyrimidine-3-carbaldehyde (50 mg, 0.10 mmol), 6-bromo-2-fluoro-pyridine-3-carbaldehyde (31 mg, 0.15 mmol) and potassium carbonate (41 mg, 0.30 mmol) in degassed dioxane/water (5:1) was added Pd(PPh₃)₄ (12 mg, 0.01 mmol) and the mixture was stirred at 90° C. for 2 hours under N₂(g). Water was added and the mixture was extracted into EtOAc three times. The combined organic layers were dried over anhydrous sodium sulfate, concentrated, and purified on silica gel column to yield 8-[3-[2-chloro-3-(6-fluoro-5-formyl-2-pyridyl)phenyl]-2-methyl-phenyl]-4-oxo-pyrido[1,2-a]pyrimidine-3-carbaldehyde (34 mg, 68.4%). MS: m/z found 498.1 [M+H]⁺.

### 8-[3-[2-Chloro-3-[6-fluoro-5-[[[(2S)-5-oxopyrrolidin-2-yl]methylamino]methyl]-2-pyridyl]phenyl]-2-methyl-phenyl]-3-[[[(2S)-5-oxopyrrolidin-2-yl]methylamino]methyl]pyrido[1,2-a]pyrimidin-4-one

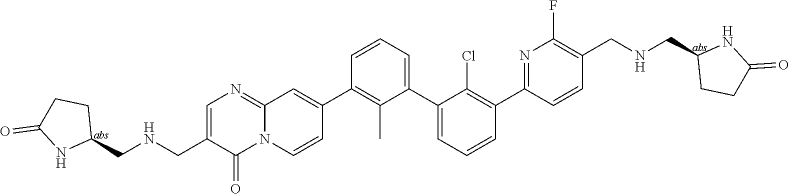 [SMILES: Cc1c(-c2ccn3c(=O)c(CNC[C@@H]4CCC(=O)N4)cnc3c2)cccc1-c1cccc(-c2ccc(CNC[C@@H]3CCC(=O)N3)c(F)n2)c1Cl]

A mixture of 8-[3-[2-chloro-3-(6-fluoro-5-formyl-2-pyridyl)phenyl]-2-methyl-phenyl]-4-oxo-pyrido[1,2-a]pyrimidine-3-carbaldehyde (34 mg, 0.07 mmol), (5S)-5-(aminomethyl)pyrrolidin-2-one (31 mg, 0.27 mmol) and acetic acid (8 uL, 8.20 mg, 0.14 mmol) in MeOH/THF (1:1) was stirred for 1 hour in an iced-water bath. NaBH₃CN (17 mg, 0.27 mmol) was added and the mixture was quenched with water after 50 min further stirring. The mixture was directly purified by HPLC to give 8-[3-[2-chloro-3-[6-fluoro-5-[[[(2S)-5-oxopyrrolidin-2-yl]methylamino]methyl]-2-pyridyl]phenyl]-2-methyl-phenyl]-3-[[[(2S)-5-oxopyrrolidin-2-yl]methylamino]methyl]pyrido[1,2-a]pyrimidin-4-one (30 mg, 63.3%). MS: m/z found 694.3 [M+H]⁺. ¹H NMR (400 MHz, Methanol-d₄): δ 9.40 (d, J=7.2 Hz, 1H), 8.81 (s, 1H), 8.37 (dd, J=9.7, 7.7 Hz, 1H), 8.08 (d, J=1.8 Hz, 1H), 8.01 (dd, J=7.2, 1.8 Hz, 1H), 7.75 (dd, J=7.6, 1.6 Hz, 1H), 7.68-7.60 (m, 1H), 7.61-7.51 (m, 3H), 7.47 (dd, J=7.5, 1.8 Hz, 1H), 7.43 (dd, J=6.9, 2.1 Hz, 1H), 4.50-4.42 (m, 4H), 4.17 (q, J=5.0 Hz, 2H), 3.43-3.33 (m, 4H), 2.53-2.36 (m, 6H), 2.19 (s, 3H), 2.05-1.92 (m, 2H).

### Example 58: 2-(2,2′-Dichloro-3′-(6-methoxy-5-(((((S)-5-oxopyrrolidin-2-yl)methyl)amino)methyl)pyrazin-2-yl)-[1,1′-biphenyl]-3-yl)-7-(((((S)-5-oxopyrrolidin-2-yl)methyl)amino)methyl)-4H-pyrido[1,2-a]pyrimidin-4-one

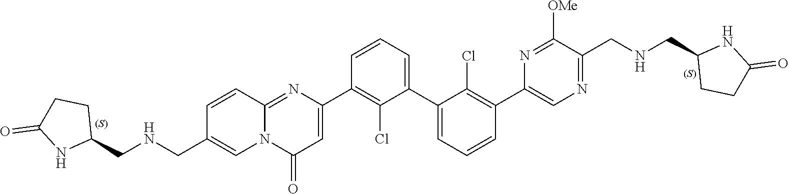

### 5-(2,2′-Dichloro-3′-(4,4,5,5-tetramethyl-1,3,2-dioxaborolan-2-yl)-[1,1′-biphenyl]-3-yl)-3-methoxypyrazine-2-carbaldehyde

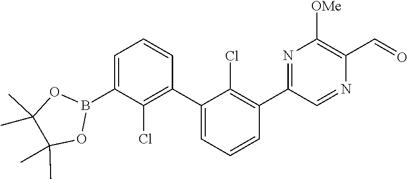 [SMILES: COc1nc(-c2cccc(-c3cccc(B4OC(C)(C)C(C)(C)O4)c3Cl)c2Cl)cnc1C=O]

In a vial charged with 2-[2-chloro-3-[2-chloro-3-(4,4,5,5-tetramethyl-1,3,2-dioxaborolan-2-yl)phenyl]phenyl]-4,4,5,5-tetramethyl-1,3,2-dioxaborolane (230 mg, 0.48 mmol), 5-bromo-3-methoxy-pyrazine-2-carbaldehyde (94 mg, 0.36 mmol), potassium carbonate (67 mg, 0.48 mmol) and Pd(PPh₃)₄ (28 mg, 0.02 mmol) was added 1,4-dioxane and water (5.5 mL, 10:1). The solution was sparged with N₂ for 5 min then heated under N₂ at 90° C. for 1.5 h. The reaction was cooled to rt, diluted with DCM (10 mL), stirred at rt for 20 min, filtered through Celite, rinsed with DCM (10 mL), and concentrated to a brown oil. The crude oil was purified by normal phase silica column chromatography from 10-60% EA/hexanes to afford 5-(2,2′-dichloro-3′-(4,4,5,5-tetramethyl-1,3,2-dioxaborolan-2-yl)-[1,1′-biphenyl]-3-yl)-3-methoxypyrazine-2-carbaldehyde (55 mg). MS: m/z found 485.1 [M+H]⁺. ¹H NMR (400 MHz, Chloroform-d) δ 10.32 (d, J=2.5 Hz, 1H), 8.77 (d, J=2.3 Hz, 1H), 7.77-7.70 (m, 1H), 7.67 (dt, J=7.5, 1.4 Hz, 1H), 7.50-7.45 (m, 1H), 7.38 (ddd, J=7.0, 2.4, 1.3 Hz, 1H), 7.36-7.32 (m, 2H), 4.18 (s, 3H), 1.38 (d, J=2.3 Hz, 12H).

### 2-(2,2′-Dichloro-3′-(5-formyl-6-methoxypyrazin-2-yl)-[1,1′-biphenyl]-3-yl)-4-oxo-4H-pyrido[1,2-a]pyrimidine-7-carbaldehyde

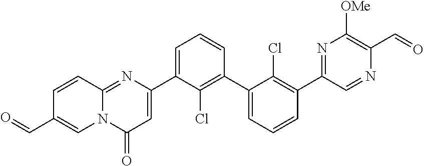 [SMILES: COc1nc(-c2cccc(-c3cccc(-c4cc(=O)n5cc(C=O)ccc5n4)c3Cl)c2Cl)cnc1C=O]

To a vial charged with 5-[2-chloro-3-[2-chloro-3-(4,4,5,5-tetramethyl-1,3,2-dioxaborolan-2-yl)phenyl]phenyl]-3-methoxy-pyrazine-2-carbaldehyde (50 mg, 0.10 mmol) was added potassium carbonate (21 mg, 0.15 mmol), Pd(PPh₃)₄ (12 mg, 0.01 mmol) and 2-chloro-4-oxo-4H-pyrido[1,2-a]pyrimidine-7-carbaldehyde (24 mg, 0.11 mmol), followed by an 8:1 mixture dioxane/water (2 mL). The brown suspension was sparged with N₂ for 5 min before heating to 95° C. for 1 h under positive N₂ pressure. The reaction was cooled to rt, diluted with DCM (3 mL) and stirred for 10 min at rt. The black suspension was filtered over Celite, rinsed with DCM (10 mL), and concentrated to dryness. The crude material was purified by flash column chromatography from 0-100% EA/hexanes to afford 2-(2,2′-dichloro-3′-(5-formyl-6-methoxypyrazin-2-yl)-[1,1′-biphenyl]-3-yl)-4-oxo-4H-pyrido[1,2-a]pyrimidine-7-carbaldehyde (22 mg). MS: m/z found 531.1 [M+H]⁺. ¹H NMR (400 MHz, Chloroform-d) δ 10.34 (d, J=0.9 Hz, 1H), 10.09 (d, J=1.0 Hz, 1H), 9.56 (d, J=2.0 Hz, 1H), 8.79 (d, J=0.9 Hz, 1H), 8.20-8.13 (m, 1H), 8.02 (s, 1H), 7.76 (dd, J=9.3, 0.9 Hz, 1H), 7.74-7.70 (m, 1H), 7.53 (tdd, J=7.6, 5.7, 1.0 Hz, 2H), 7.49-7.43 (m, 2H), 6.92 (d, J=0.9 Hz, 1H), 4.20 (d, J=0.9 Hz, 3H).

### 2-(2,2′-Dichloro-3′-(6-methoxy-5-(((((S)-5-oxopyrrolidin-2-1)methyl)amino)methyl)pyrazin-2-yl)-[1,1′-biphenyl]-3-yl)-7-(((((S)-5-oxopyrrolidin-2-yl)methyl)amino)methyl)-4H-pyrido[1,2-a]pyrimidin-4-one

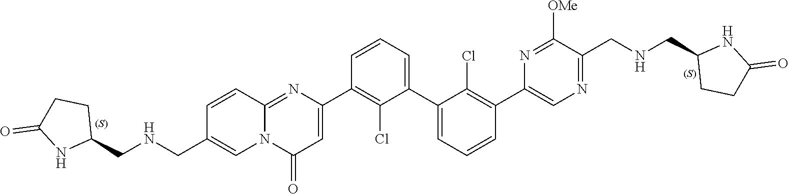

To a stirring solution of 2-[2-chloro-3-[2-chloro-3-(5-formyl-6-methoxy-pyrazin-2-yl)phenyl]phenyl]-4-oxo-pyrido[1,2-a]pyrimidine-7-carbaldehyde (20 mg, 0.04 mmol) in MeOH/THF (1:1, 2 mL) at rt was added (5S)-5-(aminomethyl)pyrrolidin-2-one (17 mg, 0.15 mmol) and acetic acid (4 uL, 0.08 mmol). After stirring for 1 h at rt, sodium cyanoborohydride (7 mg, 0.11 mmol) was added and the mixture was stirred for 16 h. The crude mixture was concentrated to dryness, dissolved in DMSO (1.5 mL) and purified by reverse phase HPLC from 0-80% MeCN/H₂O over a 10 min ramp time to afford 2-(2,2′-dichloro-3′-(6-methoxy-5-(((((S)-5-oxopyrrolidin-2-1)methyl)amino)methyl)pyrazin-2-yl)-[1,1′-biphenyl]-3-yl)-7-(((((S)-5-oxopyrrolidin-2-yl)methyl)amino)methyl)-4H-pyrido[1,2-a]pyrimidin-4-one (9 mg) as a white solid. MS: m/z found 727.4 [M+H]⁺. ¹H NMR (400 MHz, Chloroform-d) δ 9.05-9.00 (m, 1H), 8.46 (t, J=0.9 Hz, 1H), 7.86 (dd, J=9.1, 2.0 Hz, 1H), 7.75 (d, J=9.1 Hz, 1H), 7.65 (dddd, J=6.9, 5.0, 1.8, 0.8 Hz, 2H), 7.52-7.35 (m, 4H), 6.81 (d, J=0.6 Hz, 1H), 6.15 (d, J=16.9 Hz, 2H), 4.05 (d, J=0.7 Hz, 3H), 4.01 (d, J=2.8 Hz, 2H), 3.99-3.86 (m, 2H), 3.84-3.71 (m, 2H), 2.86 (ddd, J=13.4, 12.0, 4.1 Hz, 2H), 2.63 (ddd, J=24.4, 12.0, 8.5 Hz, 2H), 2.37 (dd, J=9.1, 7.3 Hz, 4H), 2.26 (ddd, J=11.8, 8.4, 6.9 Hz, 2H), 1.83-1.77 (m, 2H).

### Example 59: 2-(2,2′-Dichloro-3′-(6-methoxy-5-(((((S)-5-oxopyrrolidin-2-yl)methyl)amino)methyl)pyridin-2-yl)-[1,1′-biphenyl]-3-yl)-7-(((((S)-5-oxopyrrolidin-2-yl)methyl)amino)methyl)-4H-pyrido[1,2-a]pyrimidin-4-one

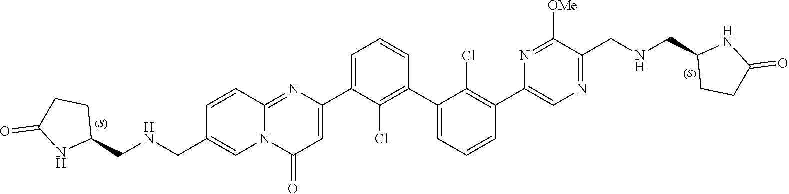

### 7-Bromo-2-hydroxy-4H-pyrido[1,2-a]pyrimidin-4-one

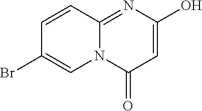 [SMILES: O=c1cc(O)nc2ccc(Br)cn12]

To a stirring yellow suspension of 5-bromopyridin-2-amine (20.0 g, 116 mmol) in CHCl₃ (250 mL) at 0° C. under N₂ was added malonyl chloride (13.5 mL, 19.6 g, 139 mmol) slowly dropwise via addition funnel over 30 min. The reaction was heated at 45° C. while stirring overnight. After 18 h stirring, the reaction was cooled to rt and diluted with DCM (100 mL). The suspension and was stirred for 1 h, filtered, rinsed with DCM (20 mL) and dried in a vacuum oven at 55° C. to afford 7-bromo-2-hydroxy-4H-pyrido[1,2-a]pyrimidin-4-one (27.0 g). MS: m/z found 241.0 [M+H]⁺. ¹H NMR (400 MHz, Methanol-d₄) δ 9.28 (dd, J=2.2, 0.7 Hz, 1H), 8.47 (dd, J=9.3, 2.2 Hz, 1H), 7.63 (dd, J=9.3, 0.7 Hz, 1H), 3.33 (s, 2H).

### 7-Bromo-2-chloro-4H-pyrido[1,2-a]pyrimidin-4-one

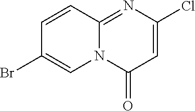 [SMILES: O=c1cc(Cl)nc2ccc(Br)cn12]

To a flask charged with 7-bromo-2-hydroxy-pyrido[1,2-a]pyrimidin-4-one (24.0 g, 100 mmol), toluene (50 mL) and equipped with reflux condenser at rt was added neat phosphoryl trichloride, (27.84 mL, 299 mmol) in a slow stream via syringe. The yellow suspension was heated to 110° C. for 18 h. The reaction mixture was cooled to 45° C. and diluted with 12 mL of toluene. Water (25 mL) was slowly added over 1 h until no obvious quenching could be detected through bubbling, off gassing, or exotherm. The solution was diluted with ethyl acetate (50 mL) and stirred while cooling to rt. The solution was filtered over a bed of Celite and rinsed with copious ethyl acetate. The biphasic mixture was separated and the organic phase was washed with sat aq NaHCO₃(2×50 mL) until bubbling subsided. The organic layer was dried over anhydrous sulfate, filtered, and concentrated to afford 7-bromo-2-chloro-4H-pyrido[1,2-a]pyrimidin-4-one (9.0 g) as a yellow solid. MS: m/z found 261.0 [M+H]⁺. ¹H NMR (400 MHz, Chloroform-d) δ 9.17 (dt, J=2.2, 0.5 Hz, 1H), 7.88 (dd, J=9.4, 2.2 Hz, 1H), 7.55 (dd, J=9.4, 0.7 Hz, 1H), 6.52 (s, 1H).

### 2-Chloro-7-vinyl-4H-pyrido[1,2-a]pyrimidin-4-one

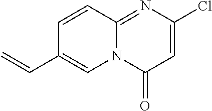

To a reaction flask charged with 7-bromo-2-chloro-4H-pyrido[1,2-a]pyrimidin-4-one (3.80 g, 14.64 mmol) was added Pd(dppf)Cl₂ (0.60 g, 0.73 mmol), trifluoro-potassio-vinyl-boron (2.35 g, 17.6 mmol), and potassium carbonate (5.06 g, 36.6 mmol) in one portion, followed by addition of THE (80 mL) and water (20 mL). The mixture was sparged with N₂ for 10 min prior to heating to reflux under N₂ atmosphere. The reaction was cooled to rt, diluted with DCM (100 mL) and stirred for 20 additional min. The reaction was filtered over a bed of Celite and rinsed with DCM (100 mL). The organic phase was washed with brine (2×50 mL), dried over anhydrous sodium sulfate, filtered and concentrated to afford crude 2-chloro-7-vinyl-4H-pyrido[1,2-a]pyrimidin-4-one. No further purification was performed and crude material was used in following oxidative cleavage. MS: m/z found 207.1 [M+H]⁺. ¹H NMR (400 MHz, Chloroform-d) δ 8.94 (d, J=2.1 Hz, 1H), 8.03 (dd, J=9.2, 2.1 Hz, 1H), 7.64 (dt, J=9.2, 0.7 Hz, 1H), 6.76 (dd, J=17.6, 10.9 Hz, 1H), 6.48 (d, J=2.1 Hz, 1H), 5.92 (d, J=17.5 Hz, 1H), 5.57 (d, J=10.9 Hz, 1H).

### 2-Chloro-4-oxo-4H-pyrido[1,2-a]pyrimidine-7-carbaldehyde

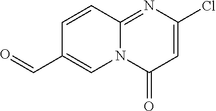 [SMILES: O=Cc1ccc2nc(Cl)cc(=O)n2c1]

To a stirring suspension of 2-chloro-7-vinyl-pyrido[1,2-a]pyrimidin-4-one (3.03 g, 14.66 mmol, 1) in a 3:1 mixture of THF/H₂O (100 mL) was added sodium periodate (9.41 g, 43.99 mmol, 3) in one portion, followed by potassium osmate dihydrate (0.24 g, 0.73 mmol) in one portion. The reaction was stirred at rt for 16 h, diluted with DCM (100 mL), and quenched by the addition of water (50 mL). The biphasic suspension separated, and the aqueous phase was extracted with DCM (2×50 mL). The combined organic layers were dried over sodium sulfate, filtered, and concentrated to afford 2-chloro-4-oxo-4H-pyrido[1,2-a]pyrimidine-7-carbaldehyde (1.1 g). MS: m/z found 209.1 [M+H]⁺. ¹H NMR (400 MHz, DMSO-d₆) δ 10.17 (s, 1H), 9.64 (s, 1H), 8.24 (d, J=9.1 Hz, 1H), 7.73 (d, J=9.0 Hz, 1H), 6.69 (s, 1H).

### 2-(2,2′-Dichloro-3′-(5-formyl-6-methoxypyridin-2-yl)-[1,1′-biphenyl]-3-yl)-4-oxo-4H-pyrido[1,2-a]pyrimidine-7-carbaldehyde

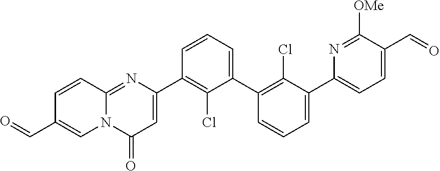 [SMILES: COc1nc(-c2cccc(-c3cccc(-c4cc(=O)n5cc(C=O)ccc5n4)c3Cl)c2Cl)ccc1C=O]

To a reaction flask charged with Pd(PPh₃)₄ (66.4 mg, 0.06 mmol), potassium carbonate (119 mg, 0.86 mmol), 2-[2-chloro-3-[2-chloro-3-(4,4,5,5-tetramethyl-1,3,2-dioxaborolan-2-yl)phenyl]phenyl]-4,4,5,5-tetramethyl-1,3,2-dioxaborolane (137 mg, 0.29 mmol), 6-chloro-2-methoxy-pyridine-3-carbaldehyde (49 mg, 0.29 mmol), and 2-chloro-4-oxo-4H-pyrido[1,2-a]pyrimidine-7-carbaldehyde (60 mg, 0.29 mmol) was added a dioxane/water mixture (4 mL, 8:1). The suspension was sparged with N₂ for 10 min, then heated at 95° C. for 1 h. The reaction was cooled to rt, diluted with DCM (5 mL) and stirred for 10 min at rt. The black solution/suspension was filtered over a Celite bed, rinsed with DCM (20 mL), and concentrated. The crude material was purified by silica flash column chromatography from 0-60% EA/hexanes to afford 2-(2,2′-dichloro-3′-(5-formyl-6-methoxypyridin-2-yl)-[1,1′-biphenyl]-3-yl)-4-oxo-4H-pyrido[1,2-a]pyrimidine-7-carbaldehyde (77.0 mg). MS: m/z found 530.1 [M+H]⁺.

### 2-(2,2′-Dichloro-3′-(6-methoxy-5-(((((S)-5-oxopyrrolidin-2-yl)methyl)amino)methyl)pyridine-2-yl)-[1,1′-biphenyl]-3-yl)-7-(((((S)-5-oxopyrrolidin-2-yl)methyl)amino)methyl)-4H-pyrido[1,2-a]pyrimidin-4-one

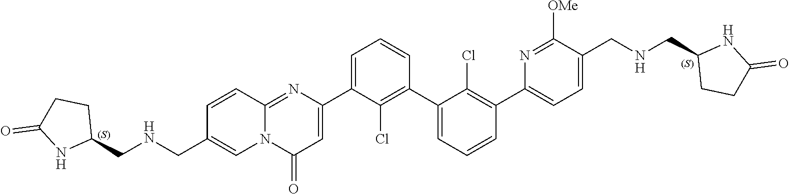

A mixture of 2-[2-chloro-3-[2-chloro-3-(5-formyl-6-methoxy-2-pyridyl)phenyl]phenyl]-4-oxo-pyrido[1,2-a]pyrimidine-7-carbaldehyde (77 mg, 0.15 mmol), (5S)-5-(aminomethyl)pyrrolidin-2-one (66 mg, 0.58 mmol), and acetic acid (17 uL, 0.29 mmol) were stirred at rt in MeOH/THF (2 mL, 1:1) for 1 h. To the reaction mixture was added sodium cyanoborohydride (27 mg, 0.44 mmol) and stirred for 16 h at rt. The solvent was removed, and the reaction mixture was concentrated to dryness. The crude material was dissolved in DMSO (1.5 mL) and purified by reverse phase HPLC from 0-60% MeCN/H₂O over 10 min ramp to afford 2-(2,2′-dichloro-3′-(6-methoxy-5-(((((S)-5-oxopyrrolidin-2-yl)methyl)amino)methyl)pyridine-2-yl)-[1,1′-biphenyl]-3-yl)-7-(((((S)-5-oxopyrrolidin-2-yl)methyl)amino)methyl)-4H-pyrido[1,2-a]pyrimidin-4-one (10 mg) as a white solid. MS: m/z found 726.3 [M+H]⁺. ¹H NMR (400 MHz, Chloroform-d) δ 9.05-9.00 (m, 1H), 7.85 (dd, J=9.2, 2.0 Hz, 1H), 7.74 (d, J=9.1 Hz, 1H), 7.66 (td, J=7.9, 1.8 Hz, 2H), 7.58 (d, J=7.5 Hz, 1H), 7.51-7.36 (m, 3H), 7.36-7.26 (m, 2H), 6.80 (d, J=0.9 Hz, 1H), 6.33 (s, 1H), 6.15 (s, 1H), 4.02 (d, J=0.9 Hz, 3H), 3.99-3.86 (m, 2H), 3.80 (s, 2H), 3.75 (s, 2H), 2.81 (ddd, J=18.6, 12.0, 4.1 Hz, 2H), 2.58 (ddd, J=15.3, 12.0, 8.3 Hz, 2H), 2.36 (dd, J=9.1, 6.7 Hz, 4H), 2.25 (ddd, J=13.9, 10.0, 6.8 Hz, 2H), 1.78 (dtd, J=12.2, 6.3, 5.9, 2.8 Hz, 2H).

### Example 60: 2,2′-(2,2′-Dichloro-[1,1′-biphenyl]-3,3′-diyl)bis(7-(((((S)-5-oxopyrrolidin-2-yl)methyl)amino)methyl)-4H-pyrido[1,2-a]pyrimidin-4-one)

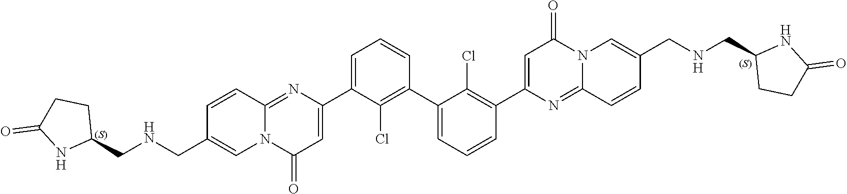

### 2,2′-(2,2′-Dichloro-[1,1′-biphenyl]-3,3′-diyl)bis(4-oxo-4H-pyrido[1,2-a]pyrimidine-7-carbaldehyde)

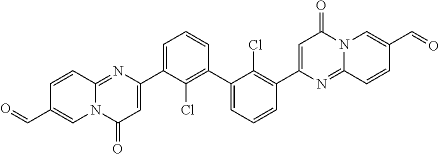 [SMILES: O=Cc1ccc2nc(-c3cccc(-c4cccc(-c5cc(=O)n6cc(C=O)ccc6n5)c4Cl)c3Cl)cc(=O)n2c1]

To a reaction flask charged with Pd(PPh₃)₄ (66 mg, 0.06 mmol), potassium carbonate (119 mg, 0.86 mmol), 2-[2-chloro-3-[2-chloro-3-(4,4,5,5-tetramethyl-1,3,2-dioxaborolan-2-yl)phenyl]phenyl]-4,4,5,5-tetramethyl-1,3,2-dioxaborolane (137 mg, 0.29 mmol), 6-chloro-2-methoxy-pyridine-3-carbaldehyde (49 mg, 0.29 mmol), and 2-chloro-4-oxo-4H-pyrido[1,2-a]pyrimidine-7-carbaldehyde (60 mg, 0.29 mmol) was added a dioxane/water mixture (4 mL, 8:1). The suspension was sparged with N₂ for 10 min, then heated at 95° C. for 1 h. The reaction was cooled to rt, diluted with DCM (5 mL) and stirred for 10 min at rt. The black solution/suspension was filtered over a Celite bed, rinsed with DCM (20 mL), and concentrated. The crude material was purified by silica flash column chromatography from 0-60% EA/hexanes to afford 2,2′-(2,2′-dichloro-[1,1′-biphenyl]-3,3′-diyl)bis(4-oxo-4H-pyrido[1,2-a]pyrimidine-7-carbaldehyde) (45 mg). MS: m/z found 567.3 [M+H]⁺.

### 2,2′-(2,2′-Dichloro-[1,1′-biphenyl]-3,3′-diyl)bis(7-(((((S)-5-oxopyrrolidin-2-yl)methyl)amino)methyl)-4H-pyrido[1,2-a]pyrimidin-4-one)

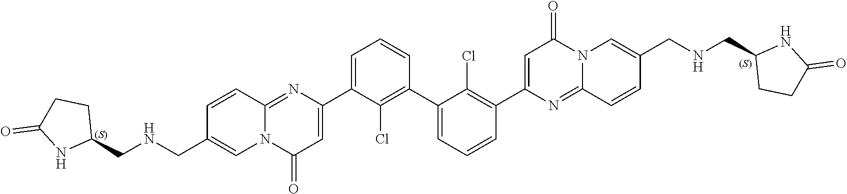

A mixture of 2,2′-(2,2′-dichloro-[1,1′-biphenyl]-3,3′-diyl)bis(4-oxo-4H-pyrido[1,2-a]pyrimidine-7-carbaldehyde) (45 mg, 0.08 mmol), (5S)-5-(aminomethyl)pyrrolidin-2-one (36 mg, 0.32 mmol), and acetic acid (9 uL, 0.16 mmol) were stirred in MeOH/THF (2 mL, 1:1) for 2 h. To the reaction mixture was added sodium cyanoborohydride (15 mg, 0.24 mmol) was added, stirred for 30 min, and concentrated to dryness. The crude material was dissolved in DMSO (1.5 mL) and purified by reverse phase HPLC from 0-60% MeCN/H₂O over 10 min ramp to afford 2,2′-(2,2′-dichloro-[1,1′-biphenyl]-3,3′-diyl)bis(7-(((((S)-5-oxopyrrolidin-2-yl)methyl)amino)methyl)-4H-pyrido[1,2-a]pyrimidin-4-one) (16.4 mg) as a white solid. MS: m/z found 763.3 [M+H]⁺. ¹H NMR (400 MHz, Chloroform-d) δ 9.03 (d, J=1.9 Hz, 2H), 7.85 (dd, J=9.2, 2.0 Hz, 2H), 7.74 (dd, J=9.1, 0.7 Hz, 2H), 7.64 (ddd, J=7.6, 1.8, 0.7 Hz, 2H), 7.46 (t, J=7.6 Hz, 2H), 7.39 (ddd, J=7.6, 1.8, 0.7 Hz, 2H), 6.78 (d, J=1.1 Hz, 2H), 6.38 (s, 2H), 4.00-3.84 (m, 4H), 3.79-3.71 (m, 2H), 2.84 (dd, J=11.9, 4.0 Hz, 2H), 2.66-2.56 (m, 2H), 2.41-2.31 (m, 4H), 2.25 (dt, J=12.7, 7.5 Hz, 2H), 1.85-1.71 (m, 2H).

### Example 61: 8-(2′-Chloro-2-methyl-3′-(5-(((((S)-5-oxopyrrolidin-2-yl)methyl)amino)methyl)pyridin-2-yl)-[1,1′-biphenyl]-3-yl)-3-(((((S)-5-oxopyrrolidin-2-yl)methyl)amino)methyl)-4H-pyrido[1,2-a]pyrimidin-4-one

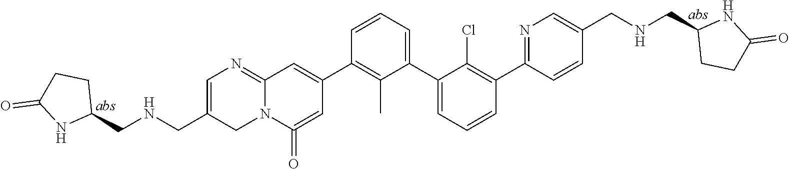

### 8-[3-[2-Chloro-3-(5-formyl-2-pyridyl)phenyl]-2-methyl-phenyl]-4-oxo-pyrido[1,2-a]pyrimidine-3-carbaldehyde

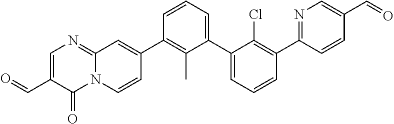 [SMILES: Cc1c(-c2ccn3c(=O)c(C=O)cnc3c2)cccc1-c1cccc(-c2ccc(C=O)cn2)c1Cl]

To a solution of 8-[3-[2-chloro-3-(4,4,5,5-tetramethyl-1,3,2-dioxaborolan-2-yl)phenyl]-2-methyl-phenyl]-4-oxo-pyrido[1,2-a]pyrimidine-3-carbaldehyde (60 mg, 0.12 mmol), 6-bromopyridine-3-carbaldehyde (33 mg, 0.18 mmol) and potassium carbonate (50 mg, 0.36 mmol) in degassed dioxane/water (5:1) was added Pd(PPh₃)₄ (14 mg, 0.01 mmol) and the mixture was stirred at 95° C. for 3 hours under N₂(g). Water was added and the mixture was extracted into EtOAc three times. The combined organic layers were dried over anhydrous sodium sulfate, concentrated, and purified on silica gel column using EtOAc in hexanes (0 to 60% gradient) to give 8-[3-[2-chloro-3-(5-formyl-2-pyridyl)phenyl]-2-methyl-phenyl]-4-oxo-pyrido[1,2-a]pyrimidine-3-carbaldehyde (35 mg, 60.9%). MS: m/z found 480.2 [M+H]⁺.

### 8-[3-[2-Chloro-3-[5-[[[(2S)-5-oxopyrrolidin-2-yl]methylamino]methyl]-2-pyridyl]phenyl]-2-methyl-phenyl]-3-[[[(2S)-5-oxopyrrolidin-2-yl]methylamino]methyl]pyrido[1,2-a]pyrimidin-4-one

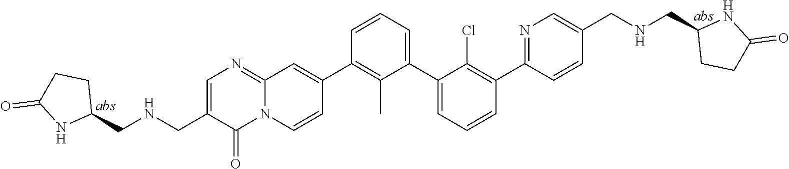 [SMILES: Cc1c(-c2ccn3c(=O)c(CNC[C@@H]4CCC(=O)N4)cnc3c2)cccc1-c1cccc(-c2ccc(CNC[C@@H]3CCC(=O)N3)cn2)c1Cl]

A mixture of 8-[3-[2-chloro-3-(5-formyl-2-pyridyl)phenyl]-2-methyl-phenyl]-4-oxo-pyrido[1,2-a]pyrimidine-3-carbaldehyde (35 mg, 0.07 mmol), (5S)-5-(aminomethyl)pyrrolidin-2-one (33 mg, 0.29 mmol), acetic acid (8. uL, 8.76 mg, 0.15 mmol) and NaBH₃CN (18 mg, 0.29 mmol) in MeOH/THF (1:1) was stirred for 1.5 hour at rt. The mixture was quenched with water and directly purified by HPLC to give 8-[3-[2-chloro-3-[5-[[[(2S)-5-oxopyrrolidin-2-yl]methylamino]methyl]-2-pyridyl]phenyl]-2-methyl-phenyl]-3-[[[(2S)-5-oxopyrrolidin-2-yl]methylamino]methyl]pyrido[1,2-a]pyrimidin-4-one (40 mg, 81.1%). MS: m/z found 676.3 [M+H]⁺. ¹H NMR (400 MHz, Methanol-d₄): δ 9.41 (d, J=7.1 Hz, 1H), 9.35 (d, J=2.1 Hz, 1H), 9.02 (dt, J=8.3, 1.8 Hz, 1H), 8.81 (s, 1H), 8.43-8.37 (m, 1H), 8.09 (d, J=1.7 Hz, 1H), 8.01 (dd, J=7.2, 1.8 Hz, 1H), 7.83 (dd, J=7.5, 1.9 Hz, 1H), 7.79-7.69 (m, 2H), 7.63-7.54 (m, 2H), 7.45 (dd, J=7.0, 2.1 Hz, 1H), 4.68 (d, J=2.6 Hz, 2H), 4.45 (d, J=2.2 Hz, 2H), 4.24-4.09 (m, 2H), 3.47-3.33 (m, 4H), 2.50-2.35 (m, 6H), 2.21 (s, 3H), 2.09-1.93 (m, 2H).

### Example 62: 8-(2′-Chloro-3′-(6-fluoro-5-(((((S)-5-oxopyrrolidin-2-yl)methyl)amino)methyl)pyridin-2-yl)-2-methyl-[1,1′-biphenyl]-3-yl)-3-(((5-oxopyrrolidin-3-yl)amino)methyl)-4H-pyrido[1,2-a]pyrimidin-4-one

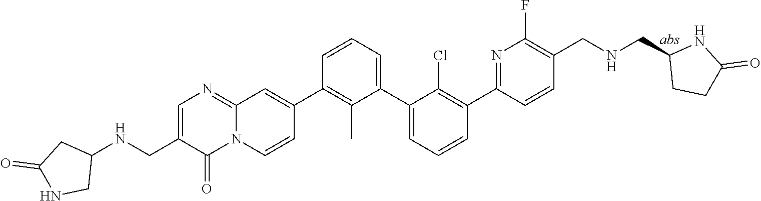 [SMILES: Cc1c(-c2ccn3c(=O)c(CNC4CNC(=O)C4)cnc3c2)cccc1-c1cccc(-c2ccc(CNC[C@@H]3CCC(=O)N3)c(F)n2)c1Cl]

### tert-Butyl N-[(6-bromo-2-fluoro-3-pyridyl)methyl]-N-[[(2S)-5-oxopyrrolidin-2-yl]methyl]carbamate

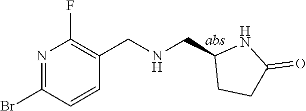 [SMILES: O=C1CC[C@@H](CNCc2ccc(Br)nc2F)N1]

A mixture of 6-bromo-2-fluoro-pyridine-3-carbaldehyde (200 mg, 0.98 mmol), (5S)-5-(aminomethyl)pyrrolidin-2-one (168 mg, 1.47 mmol), acetic acid (56 uL, 59 mg, 0.98 mmol) and NaBH₃CN (123 mg, 1.96 mmol) were stirred in MeOH/THF (1:1) for 2 hours at 0° C.

The mixture was concentrated, dissolved in EtOAc, and washed with water. The organic layer was dried over anhydrous sodium sulfate, concentrated, and used in the next step without purification. MS: m/z found 302.1 [M+H]⁺.

A mixture of (5S)-5-[[(6-bromo-2-fluoro-3-pyridyl)methylamino]methyl]pyrrolidin-2-one (210 mg, 0.70 mmol) and di-tert-butyl dicarbonate (197 mg, 0.90 mmol) in acetonitrile was stirred at rt overnight, concentrated and purified on silica gel column (MeOH in DCM, 0 to 10% gradient) to provide tert-butyl N-[(6-bromo-2-fluoro-3-pyridyl)methyl]-N-[[(2S)-5-oxopyrrolidin-2-yl]methyl]carbamate (130 mg, 46.5%). MS: m/z found 402.1 [M+H]⁺. ¹H NMR (400 MHz, Methanol-d₄): δ7.71-7.63 (m, 1H), 7.52 (d, J=7.7 Hz, 1H), 4.49 (m, 2H), 3.94 (p, J=6.3 Hz, 1H), 3.41 (m, 2H), 2.45-2.17 (m, 3H), 1.85 (br s, 1H), 1.55-1.29 (m, 9H).

### tert-Butyl N-[[6-[2-chloro-3-[3-(3-formyl-4-oxo-pyrido[1,2-a]pyrimidin-8-yl)-2-methyl-phenyl]phenyl]-2-fluoro-3-pyridyl]methyl]-N-[[(2S)-5-oxopyrrolidin-2-yl]methyl]carbamate

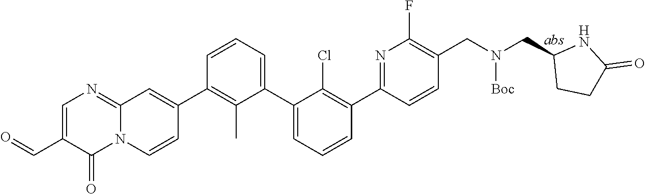 [SMILES: Cc1c(-c2ccn3c(=O)c(C=O)cnc3c2)cccc1-c1cccc(-c2ccc(CN(C[C@@H]3CCC(=O)N3)C(=O)OC(C)(C)C)c(F)n2)c1Cl]

To a solution of 8-[3-[2-chloro-3-(4,4,5,5-tetramethyl-1,3,2-dioxaborolan-2-yl)phenyl]-2-methyl-phenyl]-4-oxo-pyrido[1,2-a]pyrimidine-3-carbaldehyde (50 mg, 0.10 mmol), tert-butyl N-[(6-bromo-2-fluoro-3-pyridyl)methyl]-N-[[(2S)-5-oxopyrrolidin-2-yl]methyl]carbamate (44 mg, 0.11 mmol) and potassium carbonate (41 mg, 0.30 mmol) in degassed dioxane/water (5:1) was added Pd(PPh₃)₄ (12 mg, 0.01 mmol) and the mixture was stirred at 90° C. for 3 hours under N₂(g). Water was added and the mixture was extracted into EtOAc three times. The combined organic layers were dried over anhydrous sodium sulfate, concentrated, and purified on silica gel column using EtOAc in hexanes (0 to 100% gradient) to provide tert-butyl N-[[6-[2-chloro-3-[3-(3-formyl-4-oxo-pyrido[1,2-a]pyrimidin-8-yl)-2-methyl-phenyl]phenyl]-2-fluoro-3-pyridyl]methyl]-N-[[(2S)-5-oxopyrrolidin-2-yl]methyl]carbamate (51 mg, 73.4%). MS: m/z found 696.3 [M+H]⁺.

### tert-Butyl N-[[6-[2-chloro-3-[2-methyl-3-[4-oxo-3-[[(5-oxopyrrolidin-3-yl)amino]methyl]pyrido[1,2-a]pyrimidin-8-yl]phenyl]phenyl]-2-fluoro-3-pyridyl]methyl]-N-[[(2S)-5-oxopyrrolidin-2-yl]methyl]carbamate

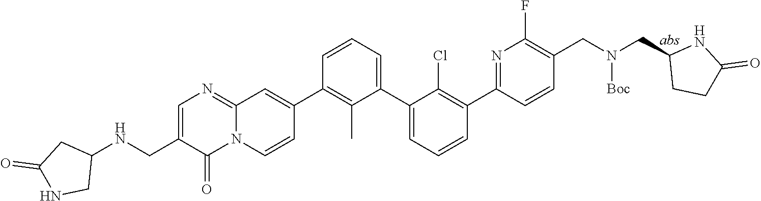 [SMILES: Cc1c(-c2ccn3c(=O)c(CNC4CNC(=O)C4)cnc3c2)cccc1-c1cccc(-c2ccc(CN(C[C@@H]3CCC(=O)N3)C(=O)OC(C)(C)C)c(F)n2)c1Cl]

A mixture of tert-butyl N-[[6-[2-chloro-3-[3-(3-formyl-4-oxo-pyrido[1,2-a]pyrimidin-8-yl)-2-methyl-phenyl]phenyl]-2-fluoro-3-pyridyl]methyl]-N-[[(2S)-5-oxopyrrolidin-2-yl]methyl]carbamate (50 mg, 0.07 mmol), 4-aminopyrrolidin-2-one (14 mg, 0.14 mmol) and acetic acid (4 uL, 4 mg, 0.07 mmol) in MeOH/THF (1:1) was stirred for 2 hour in an iced-water bath. NaBH₃CN (9 mg, 0.14 mmol) was added and the mixture was quenched with water after 2 h further stirring. The mixture was directly purified by HPLC to give tert-butyl N-[[6-[2-chloro-3-[2-methyl-3-[4-oxo-3-[[(5-oxopyrrolidin-3-yl)amino]methyl]pyrido[1,2-a]pyrimidin-8-yl]phenyl]phenyl]-2-fluoro-3-pyridyl]methyl]-N-[[(2S)-5-oxopyrrolidin-2-yl]methyl]carbamate (30 mg, 53.5%). MS: m/z found 780.4 [M+H]⁺.

### 8-[3-[2-Chloro-3-[6-fluoro-5-[[[(2S)-5-oxopyrrolidin-2-yl]methylamino]methyl]-2-pyridyl]phenyl]-2-methyl-phenyl]-3-[[(5-oxopyrrolidin-3-yl)amino]methyl]pyrido[1,2-a]pyrimidin-4-one

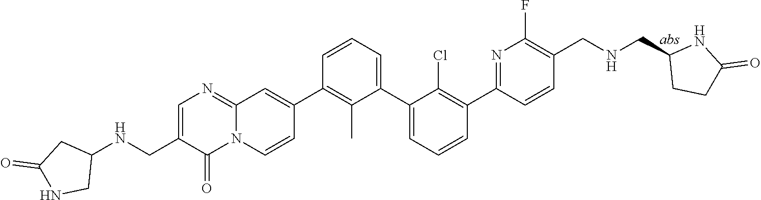 [SMILES: Cc1c(-c2ccn3c(=O)c(CNC4CNC(=O)C4)cnc3c2)cccc1-c1cccc(-c2ccc(CNC[C@@H]3CCC(=O)N3)c(F)n2)c1Cl]

tert-Butyl N-[[6-[2-chloro-3-[2-methyl-3-[4-oxo-3-[[(5-oxopyrrolidin-3-yl)amino]methyl]pyrido[1,2-a]pyrimidin-8-yl]phenyl]phenyl]-2-fluoro-3-pyridyl]methyl]-N-[[(2S)-5-oxopyrrolidin-2-yl]methyl]carbamate (30 mg, 0.04 mmol) in DCM—4M HCl in dioxane (5:1) (5 eq. HCl was used) was stirred for 45 minutes. The precipitate was filtered, washed with DCM three times and dried under vacuum to give 8-[3-[2-chloro-3-[6-fluoro-5-[[[(2S)-5-oxopyrrolidin-2-yl]methylamino]methyl]-2-pyridyl]phenyl]-2-methyl-phenyl]-3-[[(5-oxopyrrolidin-3-yl)amino]methyl]pyrido[1,2-a]pyrimidin-4-one (24 mg, 92%) as HCl salt. MS: m/z found 680.3 [M+H]⁺. ¹H NMR (400 MHz, Methanol-d₄): δ9.33 (dd, J=7.2, 0.7 Hz, 1H), 8.71 (s, 1H), 8.29 (dd, J=9.7, 7.7 Hz, 1H), 7.94 (dd, J=1.8, 0.7 Hz, 1H), 7.89-7.82 (m, 1H), 7.76 (dd, J=7.7, 1.5 Hz, 1H), 7.64 (ddd, J=7.7, 1.8, 0.7 Hz, 1H), 7.58 (td, J=7.6, 0.7 Hz, 1H), 7.55-7.50 (m, 2H), 7.47 (ddd, J=7.4, 1.8, 0.7 Hz, 1H), 7.41 (q, J=4.2 Hz, 1H), 4.46 (s, 2H), 4.41-4.37 (m, 2H), 4.30 (tt, J=8.3, 4.3 Hz, 1H), 4.09 (h, J=5.9, 5.3 Hz, 1H), 3.91 (dd, J=11.7, 7.5 Hz, 1H), 3.68 (dd, J=11.7, 3.8 Hz, 1H), 3.40-3.32 (m, 2H), 2.94 (dd, J=17.8, 8.6 Hz, 1H), 2.68 (dd, J=17.8, 4.6 Hz, 1H), 2.49-2.34 (m, 3H), 2.18 (s, 3H), 2.02-1.91 (m, 1H).

### Example 63: 8-(2,2′-Dichloro-3′-(6-methoxy-5-(((5-oxopyrrolidin-3-yl)amino)methyl)pyridin-2-yl)-[1,1′-biphenyl]-3-yl)-3-(((5-oxopyrrolidin-3-yl)amino)methyl)-4H-pyrido[1,2-a]pyrimidin-4-one

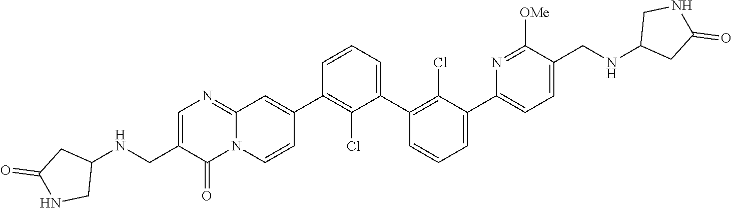 [SMILES: COc1nc(-c2cccc(-c3cccc(-c4ccn5c(=O)c(CNC6CNC(=O)C6)cnc5c4)c3Cl)c2Cl)ccc1CNC1CNC(=O)C1]

### 8-[2-Chloro-3-[2-chloro-3-(5-formyl-6-methoxy-2-pyridyl)phenyl]phenyl]-4-oxo-pyrido[1,2-a]pyrimidine-3-carbaldehyde

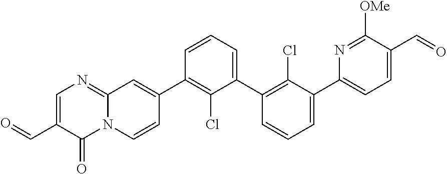 [SMILES: COc1nc(-c2cccc(-c3cccc(-c4ccn5c(=O)c(C=O)cnc5c4)c3Cl)c2Cl)ccc1C=O]

To a solution of 8-[2-chloro-3-(4,4,5,5-tetramethyl-1,3,2-dioxaborolan-2-yl)phenyl]-4-oxo-pyrido[1,2-a]pyrimidine-3-carbaldehyde (50 mg, 0.12 mmol), 6-(3-bromo-2-chloro-phenyl)-2-methoxy-pyridine-3-carbaldehyde (48 mg, 0.15 mmol) and potassium carbonate (50 mg, 0.37 mmol) in degassed dioxane/water (5:1) was added Pd(PPh₃)₄ (14 mg, 0.01 mmol) and the mixture was stirred at 90° C. for 3 hours under N₂(g). Water was added and the mixture was extracted into EtOAc three times. The combined organic layers were dried over anhydrous sodium sulfate, concentrated, and purified on silica gel column using EtOAc in hexanes (0 to 100% gradient) to generate 8-[2-chloro-3-[2-chloro-3-(5-formyl-6-methoxy-2-pyridyl)phenyl]phenyl]-4-oxo-pyrido[1,2-a]pyrimidine-3-carbaldehyde (49 mg, 75.9%). MS: m/z found 530.1 [M+H]⁺.

### 8-[2-Chloro-3-[2-chloro-3-[6-methoxy-5-[[(5-oxopyrrolidin-3-yl)amino]methyl]-2-pyridyl]phenyl]phenyl]-3-[[(5-oxopyrrolidin-3-yl)amino]methyl]pyrido[1,2-a]pyrimidin-4-one

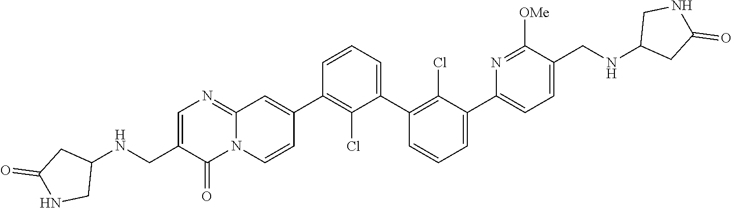 [SMILES: COc1nc(-c2cccc(-c3cccc(-c4ccn5c(=O)c(CNC6CNC(=O)C6)cnc5c4)c3Cl)c2Cl)ccc1CNC1CNC(=O)C1]

A mixture of 8-[2-chloro-3-[2-chloro-3-(5-formyl-6-methoxy-2-pyridyl)phenyl]phenyl]-4-oxo-pyrido[1,2-a]pyrimidine-3-carbaldehyde (50 mg, 0.09 mmol), 4-aminopyrrolidin-2-one (38 mg, 0.38 mmol) and acetic acid (11 uL, 11 mg, 0.19 mmol) in MeOH/THF (1:1) was stirred for 2 hour at rt. NaBH₃CN (24 mg, 0.38 mmol) was added, the mixture was quenched with water after 2 h further stirring and directly purified by HPLC to give 8-[2-chloro-3-[2-chloro-3-[6-methoxy-5-[[(5-oxopyrrolidin-3-yl)amino]methyl]-2-pyridyl]phenyl]phenyl]-3-[[(5-oxopyrrolidin-3-yl)amino]methyl]pyrido[1,2-a]pyrimidin-4-one (38 mg, 57.7%). MS: m/z found 698.3 [M+H]⁺.

### Example 64: 8-(2′-Chloro-3′-(6-(dimethylamino)-5-(((((S)-5-oxopyrrolidin-2-yl)methyl)amino)methyl)pyridin-2-yl)-2-methyl-[1,1′-biphenyl]-3-yl)-3-(((((S)-5-oxopyrrolidin-2-yl)methyl)amino)methyl)-4H-pyrido[1,2-a]pyrimidin-4-one

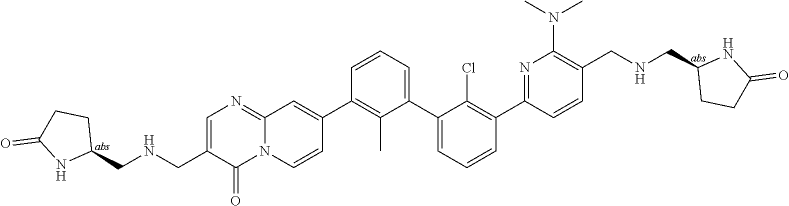 [SMILES: Cc1c(-c2ccn3c(=O)c(CNC[C@@H]4CCC(=O)N4)cnc3c2)cccc1-c1cccc(-c2ccc(CNC[C@@H]3CCC(=O)N3)c(N(C)C)n2)c1Cl]

### 8-[3-[2-Chloro-3-[6-(dimethylamino)-5-formyl-2-pyridyl]phenyl]-2-methyl-phenyl]-4-oxo-pyrido[1,2-a]pyrimidine-3-carbaldehyde

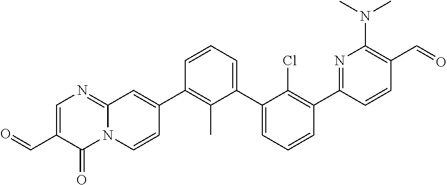 [SMILES: Cc1c(-c2ccn3c(=O)c(C=O)cnc3c2)cccc1-c1cccc(-c2ccc(C=O)c(N(C)C)n2)c1Cl]

To a solution of 8-[3-[2-chloro-3-(4,4,5,5-tetramethyl-1,3,2-dioxaborolan-2-yl)phenyl]-2-methyl-phenyl]-4-oxo-pyrido[1,2-a]pyrimidine-3-carbaldehyde (60 mg, 0.12 mmol), 6-bromo-2-(dimethylamino)pyridine-3-carbaldehyde (36 mg, 0.16 mmol) and potassium carbonate (50 mg, 0.36 mmol) in degassed dioxane/water (5:1) was added Pd(PPh₃)₄ (14 mg, 0.01 mmol) and the mixture was stirred at 90° C. for 3 hours under N₂(g). Water was added and the mixture was extracted into EtOAc three times. The combined organic layers were dried over anhydrous sodium sulfate, concentrated, and purified on silica gel column using EtOAc in hexanes (0 to 60% gradient) to give 8-[3-[2-chloro-3-[6-(dimethylamino)-5-formyl-2-pyridyl]phenyl]-2-methyl-phenyl]-4-oxo-pyrido[1,2-a]pyrimidine-3-carbaldehyde (35 mg, 55.9%). MS: m/z found 523.2 [M+H]⁺.

### 8-[3-[2-Chloro-3-[6-(dimethylamino)-5-[[[(2S)-5-oxopyrrolidin-2-yl]methylamino]methyl]-2-pyridyl]phenyl]-2-methyl-phenyl]-3-[[[(2S)-5-oxopyrrolidin-2-yl]methylamino]methyl]pyrido[1,2-a]pyrimidin-4-one

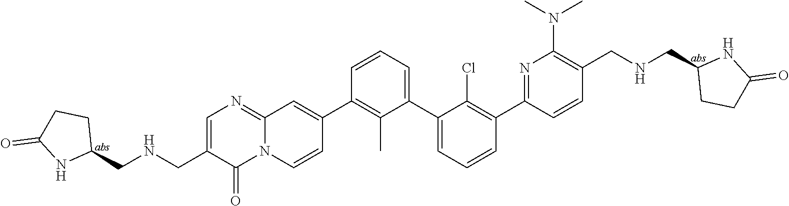 [SMILES: Cc1c(-c2ccn3c(=O)c(CNC[C@@H]4CCC(=O)N4)cnc3c2)cccc1-c1cccc(-c2ccc(CNC[C@@H]3CCC(=O)N3)c(N(C)C)n2)c1Cl]

A mixture of 8-[3-[2-chloro-3-[6-(dimethylamino)-5-formyl-2-pyridyl]phenyl]-2-methyl-phenyl]-4-oxo-pyrido[1,2-a]pyrimidine-3-carbaldehyde (35 mg, 0.07 mmol), (5S)-5-(aminomethyl)pyrrolidin-2-one (31 mg, 0.27 mmol) and acetic acid (8 uL, 8 mg, 0.13 mmol) in MeOH/THF (1:1) was stirred for 2 hour at rt. NaBH₃CN (17 mg, 0.27 mmol) was added, the mixture was stirred for additional 15 min, quenched with water and directly purified by HPLC to give 8-[3-[2-chloro-3-[6-(dimethylamino)-5-[[[(2S)-5-oxopyrrolidin-2-yl]methylamino]methyl]-2-pyridyl]phenyl]-2-methyl-phenyl]-3-[[[(2S)-5-oxopyrrolidin-2-yl]methylamino]methyl]pyrido[1,2-a]pyrimidin-4-one (38 mg, 78.9%). MS: m/z found 719.3 [M+H]⁺; ¹H NMR (400 MHz, Methanol-d₄): δ 9.10 (dd, J=7.3, 0.8 Hz, 1H), 8.39 (s, 1H), 7.81 (d, J=7.7 Hz, 1H), 7.63 (dd, J=2.0, 0.8 Hz, 1H), 7.59 (dd, J=7.6, 1.7 Hz, 1H), 7.47 (t, J=7.6 Hz, 1H), 7.45-7.39 (m, 3H), 7.32 (ddd, J=11.2, 7.0, 2.1 Hz, 2H), 7.23 (d, J=7.6 Hz, 1H), 3.95-3.79 (m, 6H), 2.88 (s, 6H), 2.78-2.62 (m, 4H), 2.36-2.20 (m, 6H), 2.11 (s, 3H), 1.87-1.75 (m, 2H).

### Example 65: 8-(2′-Chloro-2-methyl-4″-(((((S)-5-oxopyrrolidin-2-yl)methyl)amino)methyl)-[1,1′:3′,1″-terphenyl]-3-yl)-3-(((((S)-5-oxopyrrolidin-2-yl)methyl)amino)methyl)-4H-pyrido[1,2-a]pyrimidin-4-one

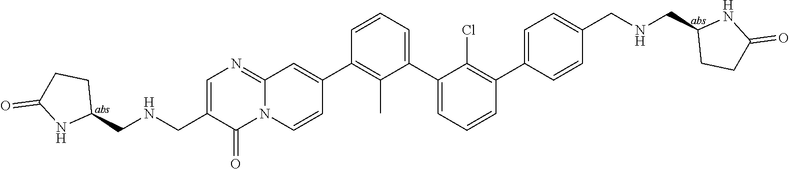 [SMILES: Cc1c(-c2ccn3c(=O)c(CNC[C@@H]4CCC(=O)N4)cnc3c2)cccc1-c1cccc(-c2ccc(CNC[C@@H]3CCC(=O)N3)cc2)c1Cl]

### 8-[3-[2-Chloro-3-(4-formylphenyl)phenyl]-2-methyl-phenyl]-4-oxo-pyrido[1,2-a]pyrimidine-3-carbaldehyde

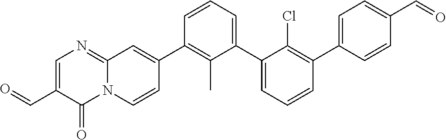 [SMILES: Cc1c(-c2ccn3c(=O)c(C=O)cnc3c2)cccc1-c1cccc(-c2ccc(C=O)cc2)c1Cl]

To a solution of 8-[3-[2-chloro-3-(4,4,5,5-tetramethyl-1,3,2-dioxaborolan-2-yl)phenyl]-2-methyl-phenyl]-4-oxo-pyrido[1,2-a]pyrimidine-3-carbaldehyde (50 mg, 0.10 mmol), 4-bromobenzaldehyde (28 mg, 0.15 mmol) and potassium carbonate (41 mg, 0.30 mmol) in degassed dioxane/water (5:1) was added Pd(PPh₃)₄ (12 mg, 0.01 mmol) and the mixture was stirred at 90° C. for 3 hours under N₂(g). Water was added and the mixture was extracted into EtOAc three times. The combined organic layers were dried over anhydrous sodium sulfate, concentrated, and purified on silica gel column using EtOAc in hexanes (0 to 60% gradient) to provide 8-[3-[2-chloro-3-(4-formylphenyl)phenyl]-2-methyl-phenyl]-4-oxo-pyrido[1,2-a]pyrimidine-3-carbaldehyde (23 mg, 48.1%). MS: m/z found 479.2 [M+H]⁺.

### 8-[3-[2-Chloro-3-[4-[[[(2S)-5-oxopyrrolidin-2-yl]methylamino]methyl]phenyl]phenyl]-2-methyl-phenyl]-3-[[[(2S)-5-oxopyrrolidin-2-yl]methylamino]methyl]pyrido[1,2-a]pyrimidin-4-one

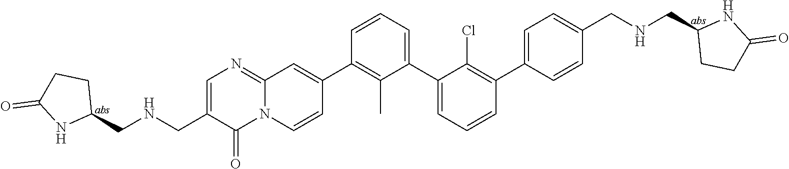 [SMILES: Cc1c(-c2ccn3c(=O)c(CNC[C@@H]4CCC(=O)N4)cnc3c2)cccc1-c1cccc(-c2ccc(CNC[C@@H]3CCC(=O)N3)cc2)c1Cl]

A mixture of 8-[3-[2-chloro-3-(4-formylphenyl)phenyl]-2-methyl-phenyl]-4-oxo-pyrido[1,2-a]pyrimidine-3-carbaldehyde (23 mg, 0.05 mmol), (5S)-5-(aminomethyl)pyrrolidin-2-one (22 mg, 0.19 mmol) and acetic acid (5 uL, 6 mg, 0.10 mmol) in MeOH/THF (1:1) was stirred for 2 hour at rt. NaBH₃CN (12 mg, 0.19 mmol) was added, the mixture was stirred for additional 15 min, quenched with water and directly purified by HPLC to give 8-[3-[2-chloro-3-[4-[[[(2S)-5-oxopyrrolidin-2-yl]methylamino]methyl]phenyl]phenyl]-2-methyl-phenyl]-3-[[[(2S)-5-oxopyrrolidin-2-yl]methylamino]methyl]pyrido[1,2-a]pyrimidin-4-one (25 mg, 77.1%). MS: m/z found 675.3 [M+H]⁺; ¹H NMR (400 MHz, Methanol-d₄): δ 9.38 (dd, J=7.2, 0.6 Hz, 1H), 8.75 (s, 1H), 8.01 (dd, J=1.8, 0.7 Hz, 1H), 7.96 (dd, J=7.2, 1.8 Hz, 1H), 7.66 (d, J=8.3 Hz, 2H), 7.62-7.57 (m, 2H), 7.56-7.50 (m, 3H), 7.47-7.40 (m, 2H), 7.38 (dd, J=7.5, 1.8 Hz, 1H), 4.43 (d, J=2.8 Hz, 2H), 4.35 (d, J=2.3 Hz, 2H), 4.16-4.02 (m, 2H), 3.40-3.19 (m, 4H), 2.48-2.34 (m, 6H), 2.19 (s, 3H), 2.00-1.88 (m, 2H).

### Example 66: 8-(2′-Chloro-3″-methoxy-2-methyl-4″-(((((S)-5-oxopyrrolidin-2-yl)methyl)amino)methyl)-[1,1′:3′,1″-terphenyl]-3-yl)-3-(((((S)-5-oxopyrrolidin-2-yl)methyl)amino)methyl)-4H-pyrido[1,2-a]pyrimidin-4-one

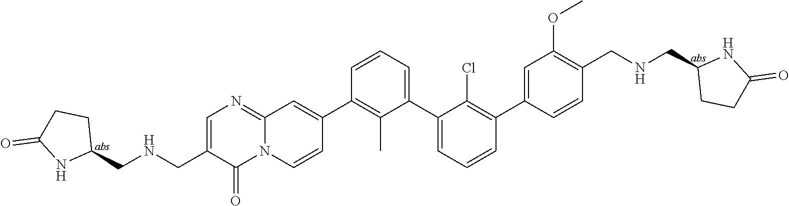 [SMILES: COc1cc(-c2cccc(-c3cccc(-c4ccn5c(=O)c(CNC[C@@H]6CCC(=O)N6)cnc5c4)c3C)c2Cl)ccc1CNC[C@@H]1CCC(=O)N1]

### 8-[3-[2-Chloro-3-(4-formyl-3-methoxy-phenyl)phenyl]-2-methyl-phenyl]-4-oxo-pyrido[1,2-a]pyrimidine-3-carbaldehyde

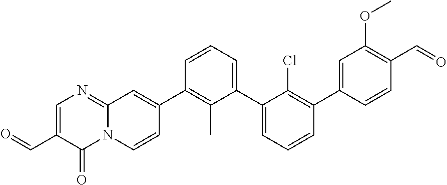 [SMILES: COc1cc(-c2cccc(-c3cccc(-c4ccn5c(=O)c(C=O)cnc5c4)c3C)c2Cl)ccc1C=O]

To a solution of 8-[3-[2-chloro-3-(4,4,5,5-tetramethyl-1,3,2-dioxaborolan-2-yl)phenyl]-2-methyl-phenyl]-4-oxo-pyrido[1,2-a]pyrimidine-3-carbaldehyde (50 mg, 0.10 mmol), 4-bromo-2-methoxy-benzaldehyde (32 mg, 0.15 mmol) and potassium carbonate (41 mg, 0.30 mmol) in degassed dioxane/water (5:1) was added Pd(PPh₃)₄ (12 mg, 0.01 mmol) and the mixture was stirred at 90° C. for 3 hours under N₂(g). Water was added and the mixture was extracted into EtOAc three times. The combined organic layers were dried over anhydrous sodium sulfate, concentrated, and purified on silica gel column using EtOAc in hexanes (0 to 60% gradient) to give 8-[3-[2-chloro-3-(4-formyl-3-methoxy-phenyl)phenyl]-2-methyl-phenyl]-4-oxo-pyrido[1,2-a]pyrimidine-3-carbaldehyde (30 mg, 59.0%). MS: m/z found 509.2 [M+H]⁺.

### 8-[3-[2-Chloro-3-[3-methoxy-4-[[[(2S)-5-oxopyrrolidin-2-yl]methylamino]methyl]phenyl]phenyl]-2-methyl-phenyl]-3-[[[(2S)-5-oxopyrrolidin-2-yl]methylamino]methyl]pyrido[1,2-a]pyrimidin-4-one

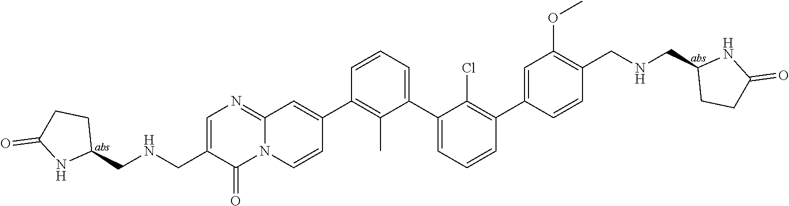 [SMILES: COc1cc(-c2cccc(-c3cccc(-c4ccn5c(=O)c(CNC[C@@H]6CCC(=O)N6)cnc5c4)c3C)c2Cl)ccc1CNC[C@@H]1CCC(=O)N1]

A mixture of 8-[3-[2-chloro-3-(4-formyl-3-methoxy-phenyl)phenyl]-2-methyl-phenyl]-4-oxo-pyrido[1,2-a]pyrimidine-3-carbaldehyde (30 mg, 0.06 mmol), (5S)-5-(aminomethyl)pyrrolidin-2-one (27 mg, 0.24 mmol) and acetic acid (7 uL, 7 mg, 0.12 mmol) in MeOH/THF (1:1) was stirred for 2 hour at rt. NaBH₃CN (15 mg, 0.24 mmol) was added, the mixture was stirred for additional 15 min, quenched with water and directly purified by HPLC to give 8-[3-[2-chloro-3-[3-methoxy-4-[[[(2S)-5-oxopyrrolidin-2-yl]methylamino]methyl]phenyl]phenyl]-2-methyl-phenyl]-3-[[[(2S)-5-oxopyrrolidin-2-yl]methylamino]methyl]pyrido[1,2-a]pyrimidin-4-one (28 mg, 67.4%). MS: m/z found 705.4 [M+H]⁺; ¹H NMR (400 MHz, Methanol-d₄): δ 9.13 (dd, J=7.5, 0.7 Hz, 1H), 8.41 (s, 1H), 7.65 (dd, J=1.9, 0.7 Hz, 1H), 7.51-7.40 (m, 5H), 7.36 (d, J=7.6 Hz, 1H), 7.34-7.30 (m, 2H), 7.07 (d, J=1.6 Hz, 1H), 7.02 (dd, J=7.6, 1.5 Hz, 1H), 3.92-3.89 (m, 5H), 3.88-3.82 (m, 4H), 2.74 (qdd, J=12.2, 7.1, 5.2 Hz, 4H), 2.37-2.24 (m, 6H), 1.82 (dddd, J=14.9, 9.2, 7.0, 4.9 Hz, 2H).

### Example 67: 8-(2′,3″-Dichloro-2-methyl-4″-(((((S)-5-oxopyrrolidin-2-yl)methyl)amino)methyl)-[1,1′:3′,1″-terphenyl]-3-yl)-3-(((((S)-5-oxopyrrolidin-2-yl)methyl)amino)methyl)-4H-pyrido[1,2-a]pyrimidin-4-one

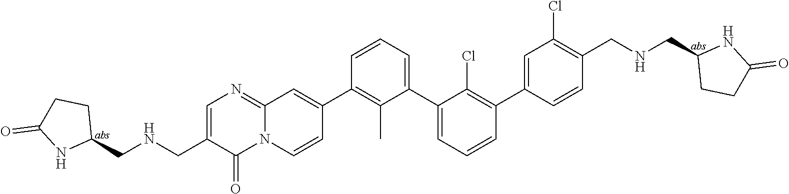 [SMILES: Cc1c(-c2ccn3c(=O)c(CNC[C@@H]4CCC(=O)N4)cnc3c2)cccc1-c1cccc(-c2ccc(CNC[C@@H]3CCC(=O)N3)c(Cl)c2)c1Cl]

### 8-[3-[2-Chloro-3-(3-chloro-4-formyl-phenyl)phenyl]-2-methyl-phenyl]-4-oxo-pyrido[1,2-a]pyrimidine-3-carbaldehyde

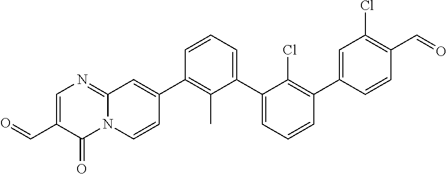 [SMILES: Cc1c(-c2ccn3c(=O)c(C=O)cnc3c2)cccc1-c1cccc(-c2ccc(C=O)c(Cl)c2)c1Cl]

To a solution of 8-[3-[2-chloro-3-(4,4,5,5-tetramethyl-1,3,2-dioxaborolan-2-yl)phenyl]-2-methyl-phenyl]-4-oxo-pyrido[1,2-a]pyrimidine-3-carbaldehyde (50 mg, 0.10 mmol), 4-bromo-2-chloro-benzaldehyde (28 mg, 0.15 mmol) and potassium carbonate (41 mg, 0.30 mmol) in degassed dioxane/water (5:1) was added Pd(PPh₃)₄ (12 mg, 0.01 mmol) and the mixture was stirred at 90° C. for 3 hours under N₂(g). Water was added and the mixture was extracted into EtOAc three times. The combined organic layers were dried over anhydrous sodium sulfate, concentrated, and purified on silica gel column using EtOAc in hexanes (0 to 60% gradient) to give 8-[3-[2-chloro-3-(3-chloro-4-formyl-phenyl)phenyl]-2-methyl-phenyl]-4-oxo-pyrido[1,2-a]pyrimidine-3-carbaldehyde (25 mg, 48.8%). MS: m/z found 513.2 [M+H]⁺.

### 8-[3-[2-Chloro-3-[3-chloro-4-[[[(2S)-5-oxopyrrolidin-2-yl]methylamino]methyl]phenyl]phenyl]-2-methyl-phenyl]-3-[[[(2S)-5-oxopyrrolidin-2-yl]methylamino]methyl]pyrido[1,2-a]pyrimidin-4-one

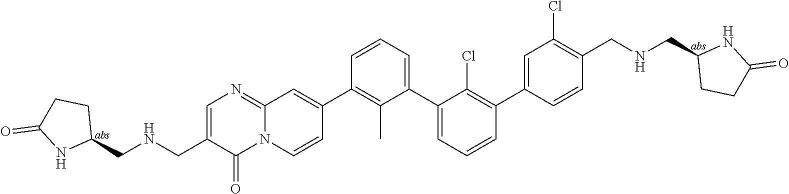 [SMILES: Cc1c(-c2ccn3c(=O)c(CNC[C@@H]4CCC(=O)N4)cnc3c2)cccc1-c1cccc(-c2ccc(CNC[C@@H]3CCC(=O)N3)c(Cl)c2)c1Cl]

A mixture of 8-[3-[2-chloro-3-(3-chloro-4-formyl-phenyl)phenyl]-2-methyl-phenyl]-4-oxo-pyrido[1,2-a]pyrimidine-3-carbaldehyde (23 mg, 0.04 mmol), (5S)-5-(aminomethyl)pyrrolidin-2-one (20 mg, 0.18 mmol) and acetic acid (5 uL, 5 mg, 0.09 mmol) in MeOH/THF (1:1) was stirred for 2 hour at rt. NaBH₃CN (11 mg, 0.18 mmol) was added, the mixture was stirred for additional 10 min, quenched with water and directly purified by HPLC to give 8-[3-[2-chloro-3-[3-chloro-4-[[[(2S)-5-oxopyrrolidin-2-yl]methylamino]methyl]phenyl]phenyl]-2-methyl-phenyl]-3-[[[(2S)-5-oxopyrrolidin-2-yl]methylamino]methyl]pyrido[1,2-a]pyrimidin-4-one (24 mg, 75.5%). MS: m/z found 709.2 [M+H]⁺; ¹H NMR (400 MHz, Methanol-d₄): δ 9.43-9.36 (m, 1H), 8.80 (s, 1H), 8.06 (t, J=1.1 Hz, 1H), 8.01 (dd, J=7.3, 1.8 Hz, 1H), 7.83 (d, J=8.0 Hz, 1H), 7.68 (d, J=1.7 Hz, 1H), 7.58-7.51 (m, 4H), 7.47 (ddd, J=7.7, 1.8, 0.5 Hz, 1H), 7.44-7.42 (m, 1H), 7.42-7.40 (m, 1H), 4.54 (d, J=1.8 Hz, 2H), 4.45 (d, J=2.2 Hz, 2H), 4.14 (h, J=5.4 Hz, 2H), 3.42-3.33 (m, 4H), 2.50-2.33 (m, 6H), 2.19 (s, 3H), 2.04-1.92 (m, 2H).

### Example 68: 8-(2′-Chloro-2-methyl-3′-(8-methyl-5-((((S)-5-oxopyrrolidin-2-yl)methyl)amino)-5,6,7,8-tetrahydro-1,8-naphthyridin-2-yl)-[1,1′-biphenyl]-3-yl)-3-(((((S)-5-oxopyrrolidin-2-yl)methyl)amino)methyl)-4H-pyrido[1,2-a]pyrimidin-4-one

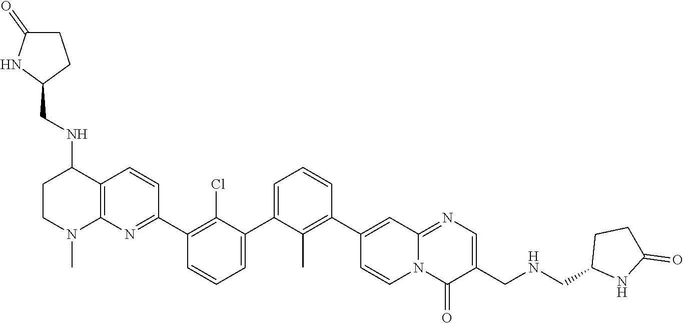 [SMILES: Cc1c(-c2ccn3c(=O)c(CNC[C@@H]4CCC(=O)N4)cnc3c2)cccc1-c1cccc(-c2ccc3c(n2)N(C)CCC3NC[C@@H]2CCC(=O)N2)c1Cl]

### 8-(2-Methyl-3-(4,4,5,5-tetramethyl-1,3,2-dioxaborolan-2-yl)-phenyl)-4-oxo-4H-pyrido[1,2-a]-pyrimidine-3-carbaldehyde

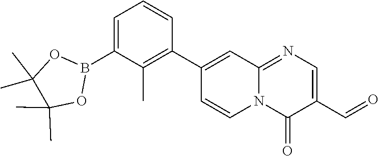 [SMILES: Cc1c(B2OC(C)(C)C(C)(C)O2)cccc1-c1ccn2c(=O)c(C=O)cnc2c1]

8-(3-Bromo-2-methyl-phenyl)-4-oxo-pyrido[1,2-a]pyrimidine-3-carbaldehyde (1.20 g, 3.15 mmol), 4,4,5,5-tetramethyl-2-(4,4,5,5-tetramethyl-1,3,2-dioxaborolan-2-yl)-1,3,2-dioxaborolane (1.04, 4.09 mmol), 1,1′-bis(diphenylphosphino)-ferrocene-palladium(ii) dichloride dichloromethane complex (0.26, 0.31 mmol) and potassium acetate (0.86 g, 8.81 mmol) were suspended in 20 ml dioxane in a sealed tube. Nitrogen gas was bubbled through the reaction solution for 2 minutes, then the vessel was sealed and heated at 95° C. for 6 hours. The reaction was cooled to rt and the mixture was filtered through CELITE®. The filtrate was concentrated and the crude oil was purified by silica gel chromatography, eluting with 0-45% EtOAc gradient in hexane to afford 8-[2-methyl-3-(4,4,5,5-tetramethyl-1,3,2-dioxaborolan-2-yl)phenyl]-4-oxo-pyrido[1,2-a]pyrimidine-3-carbaldehyde (0.90 mg, 73%) yield as a yellow oil. MS: m/z found 391.15 [M+H]⁺.

### 8-(3′-Bromo-2′-chloro-2-methyl-[1,1′-biphenyl]-3-yl)-4-oxo-4H-pyrido[1,2-a]-pyrimidine-3-carbaldehyde

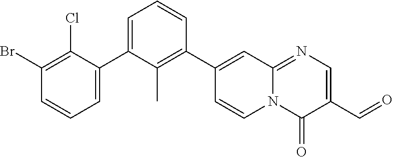 [SMILES: Cc1c(-c2ccn3c(=O)c(C=O)cnc3c2)cccc1-c1cccc(Br)c1Cl]

8-[2-Methyl-3-(4,4,5,5-tetramethyl-1,3,2-dioxaborolan-2-yl)phenyl]-4-oxo-pyrido[1,2-a]pyrimidine-3-carbaldehyde (0.93 g, 2.38 mmol), 1,3-dibromo-2-chloro-benzene (1.28 g, 4.76 mmol), 1,1′-bis(diphenylphosphino)-ferrocene-palladium(ii) dichloride dichloromethane complex (0.19 g, 0.24 mmol) and potassium carbonate (0.82 g, 5.95 mmol) were suspended in 20 ml of 9:1 dioxane/water solvent mix in a sealed tube. Nitrogen gas was bubbled through the reaction solution for 2 minutes, then the vessel was sealed and heated at 95° C. for 4 hours. The reaction was cooled to rt and the mixture was diluted with 50 ml water, then extracted with EtOAc (3×30 ml). The combined organics were concentrated and the crude oil was purified by silica gel chromatography, eluting with 0-40% EtOAc gradient in hexane to afford 8-[3-(3-bromo-2-chloro-phenyl)-2-methyl-phenyl]-4-oxo-pyrido[1,2-a]pyrimidine-3-carbaldehyde (0.32 g, 30% yield) as a light yellow solid. MS: m/z found 453, 455 [M+H]⁺.

### 8-(2′-Chloro-2-methyl-3′-(4,4,5,5-tetramethyl-1,3,2-dioxaborolan-2-yl)-[1,1′-biphenyl]-3-yl)-4-oxo-4H-pyrido[1,2-a]-pyrimidine-3-carbaldehyde

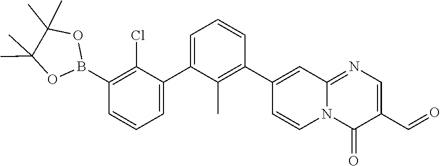 [SMILES: Cc1c(-c2ccn3c(=O)c(C=O)cnc3c2)cccc1-c1cccc(B2OC(C)(C)C(C)(C)O2)c1Cl]

8-[3-(3-Bromo-2-chloro-phenyl)-2-methyl-phenyl]-4-oxo-pyrido[1,2-a]pyrimidine-3-carbaldehyde (0.33 g, 0.65 mmol), 4,4,5,5-tetramethyl-2-(4,4,5,5-tetramethyl-1,3,2-dioxaborolan-2-yl)-1,3,2-dioxaborolane (0.21 g, 0.85 mmol), 1,1′-bis(diphenylphosphino)-ferrocene-palladium(II) dichloride dichloromethane complex (53 mg, 0.07 mmol) and potassium acetate (0.18 g, 1.83 mmol) were suspended in 6 ml dioxane in a sealed tube. Nitrogen gas was bubbled through the reaction solution for 2 minutes, then the vessel was sealed and heated at 95° C. for 2 hours. The reaction was cooled to rt and the mixture was filtered through CELITE®. The filtrate was concentrated and the crude oil was purified by silica gel chromatography, eluting with 0-45% EtOAc gradient in hexane to afford 8-[3-[2-chloro-3-(4,4,5,5-tetramethyl-1,3,2-dioxaborolan-2-yl)phenyl]-2-methyl-phenyl]-4-oxo-pyrido[1,2-a]pyrimidine-3-carbaldehyde (0.3 g, 69% yield) as a yellow oil. MS: m/z found 501, 503 [M+H]⁺.

### tert-Butyl (7-(2-chloro-3′-(3-formyl-4-oxo-4H-pyrido[1,2-a]pyrimidin-8-yl)-2′-methyl-[1,1′-biphenyl]-3-yl)-1-methyl-1,2,3,4-tetrahydro-1,8-naphthyridin-4-yl)(((S)-5-oxopyrrolidin-2-yl)methyl)carbamate

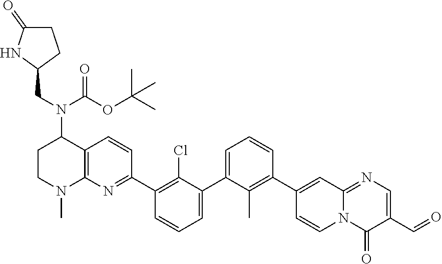 [SMILES: Cc1c(-c2ccn3c(=O)c(C=O)cnc3c2)cccc1-c1cccc(-c2ccc3c(n2)N(C)CCC3N(C[C@@H]2CCC(=O)N2)C(=O)OC(C)(C)C)c1Cl]

Pd(PPh₃)₄ (29 mg, 0.03 mmol), potassium carbonate (53 mg, 0.38 mmol),8-[3-[2-chloro-3-(4,4,5,5-tetramethyl-1,3,2-dioxaborolan-2-yl)phenyl]-2-methyl-phenyl]-4-oxo-pyrido[1,2-a]pyrimidine-3-carbaldehyde (83 mg, 0.17 mmol) and tert-butyl N-(7-bromo-1-methyl-3,4-dihydro-2H-1,8-naphthyridin-4-yl)-N-[[(2S)-5-oxopyrrolidin-2-yl]methyl]carbamate (56 mg, 0.13 mmol) were suspended in 2 ml of 4:1 dioxane/water solvent mixture, and the reaction was heated at 105° C. for 45 minutes. The reaction was cooled to rt, filtered through CELITE®, and the filtrate was concentrated. Crude product was purified by silica gel chromatography, eluting with 0-10% MeOH gradient in DCM to afford tert-butyl N-[7-[2-chloro-3-[3-(3-formyl-4-oxo-pyrido[1,2-a]pyrimidin-8-yl)-2-methyl-phenyl]phenyl]-1-methyl-3,4-dihydro-2H-1,8-naphthyridin-4-yl]-N-[[(2S)-5-oxopyrrolidin-2-yl]methyl]carbamate (125 mg, 80% yield) as a yellow oil. MS: m/z found 733.4, 735.4 [M+H]⁺.

### tert-Butyl (7-(2-chloro-2′-methyl-3′-(4-oxo-3-(((((S)-5-oxopyrrolidin-2-yl)methyl)amino)methyl)-4H-pyrido[1,2-a]pyrimidin-8-yl)-[1,1′-biphenyl]-3-yl)-1-methyl-1,2,3,4-tetrahydro-1,8-naphthyridin-4-yl)(((S)-5-oxopyrrolidin-2-yl)methyl)carbamate

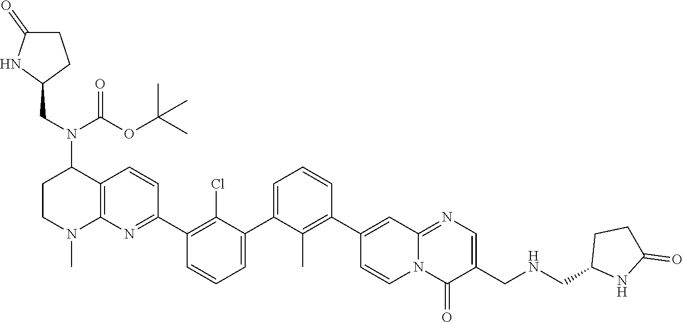 [SMILES: Cc1c(-c2ccn3c(=O)c(CNC[C@@H]4CCC(=O)N4)cnc3c2)cccc1-c1cccc(-c2ccc3c(n2)N(C)CCC3N(C[C@@H]2CCC(=O)N2)C(=O)OC(C)(C)C)c1Cl]

tert-Butyl N-[7-[2-chloro-3-[3-(3-formyl-4-oxo-pyrido[1,2-a]pyrimidin-8-yl)-2-methyl-phenyl]phenyl]-1-methyl-3,4-dihydro-2H-1,8-naphthyridin-4-yl]-N-[[(2S)-5-oxopyrrolidin-2-yl]methyl]carbamate (41 mg, 0.03 mmol), (5S)-5-(aminomethyl)pyrrolidin-2-one (6 mg, 0.05 mmol) and acetic acid (2 mg, 0.04 mmol) were dissolved in 1 ml of 1:1 MeOH/THF solvent mix, and the reaction was stirred at 25° C. for 3 hours. NaBH₃CN (3 mg, 0.05 mmol) was then added portion-wise, and the resulting mixture was stirred at 25° C. for 1 hour. The reaction was subsequently diluted with water (2 ml) and extracted with EtOAc (3×2 ml). The combined organics were further washed with brine, dried over sodium sulfate, then filtered and concentrated to afford tert-butyl N-[7-[2-chloro-3-[2-methyl-3-[4-oxo-3-[[[(2S)-5-oxopyrrolidin-2-yl]methylamino]methyl]pyrido[1,2-a]pyrimidin-8-yl]phenyl]phenyl]-1-methyl-3,4-dihydro-2H-1,8-naphthyridin-4-yl]-N-[[(2S)-5-oxopyrrolidin-2-yl]methyl]carbamate (28 mg) as a brown oil. Material was used as—is for the next step. MS: m/z found 831.5, 833.45 [M+H]⁺.

### 8-(2′-Chloro-2-methyl-3′-(8-methyl-5-((((S)-5-oxopyrrolidin-2-yl)methyl)amino)-5,6,7,8-tetrahydro-1,8-naphthyridin-2-yl)-[1,1′-biphenyl]-3-yl)-3-(((((S)-5-oxopyrrolidin-2-yl)methyl)amino)methyl)-4H-pyrido[1,2-a]pyrimidin-4-one

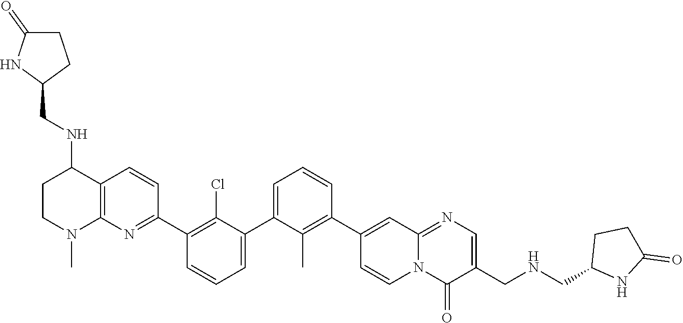 [SMILES: Cc1c(-c2ccn3c(=O)c(CNC[C@@H]4CCC(=O)N4)cnc3c2)cccc1-c1cccc(-c2ccc3c(n2)N(C)CCC3NC[C@@H]2CCC(=O)N2)c1Cl]

tert-Butyl N-[7-[2-chloro-3-[2-methyl-3-[4-oxo-3-[[[(2S)-5-oxopyrrolidin-2-yl]methylamino]methyl]pyrido[1,2-a]pyrimidin-8-yl]phenyl]phenyl]-1-methyl-3,4-dihydro-2H-1,8-naphthyridin-4-yl]-N-[[(2S)-5-oxopyrrolidin-2-yl]methyl]carbamate (26 mg, 0.03 mmol) was dissolved in 0.5 ml DCM and 4M HCl solution in dioxane (39 μL, 0.16 mmol) was added. The resulting mixture was stirred at rt for 20 minutes and the precipitate was filtered and further washed with DCM (5 ml). The solid was then dissolved in 2 ml water and the pH was adjusted to pH=9 by slow addition of saturated aqueous sodium bicarbonate solution. The aqueous solution was extracted with DCM (3×1 ml) and the combined organics were washed with brine, dried over sodium sulfate, filtered and concentrated to afford 8-[3-[2-chloro-3-[8-methyl-5-[[(2S)-5-oxopyrrolidin-2-yl]methylamino]-6,7-dihydro-5H-1,8-naphthyridin-2-yl]phenyl]-2-methyl-phenyl]-3-[[[(2S)-5-oxopyrrolidin-2-yl]methylamino]methyl]pyrido[1,2-a]pyrimidin-4-one (4.8 mg, 21% yield) as a clear oil. MS: m/z found 731, 733 [M+H]⁺. ¹H NMR (400 MHz, Chloroform-d) δ 9.08 (d, J=7.3 Hz, 1H), 8.32 (s, 1H), 7.65 (d, J=9.7 Hz, 2H), 7.40 (t, J=7.4 Hz, 3H), 7.33 (t, J=5.9 Hz, 3H), 7.24-7.21 (m, 1H), 6.86 (dd, J=7.3, 3.0 Hz, 1H), 6.18 (s, 1H), 6.01 (s, 1H), 3.91-3.81 (m, 2H), 3.76 (d, J=12.5 Hz, 4H), 3.53 (dd, J=19.6, 9.3 Hz, 2H), 3.30 (d, J=13.6 Hz, 1H), 3.19 (d, J=2.4 Hz, 3H), 2.94 (d, J=11.5 Hz, 1H), 2.81 (dd, J=12.0, 4.1 Hz, 1H), 2.59 (dt, J=25.2, 10.0 Hz, 3H), 2.36 (q, J=8.4 Hz, 5H), 2.25 (td, J=11.6, 7.5 Hz, 3H), 1.95 (d, J=4.9 Hz, 1H), 1.77 (dq, J=13.3, 6.8, 6.2 Hz, 3H). (racemic compound).

### Example 69: N-(2-(((6-(3′-(3-(((2-Acetamidoethyl)amino)methyl)-4-oxo-4H-pyrido[1,2-a]pyrimidin-8-yl)-2,2′-dichloro-[1,1′-biphenyl]-3-yl)-2-methoxypyridin-3-yl)methyl)amino)ethyl)acetamide

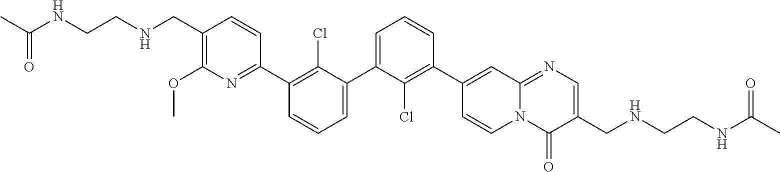 [SMILES: COc1nc(-c2cccc(-c3cccc(-c4ccn5c(=O)c(CNCCNC(C)=O)cnc5c4)c3Cl)c2Cl)ccc1CNCCNC(C)=O]

### 8-(2,2′-Dichloro-3′-(4,4,5,5-tetramethyl-1,3,2-dioxaborolan-2-yl)-[1,1′-biphenyl]-3-yl)-4-oxo-4H-pyrido[1,2-a]-pyrimidine-3-carbaldehyde

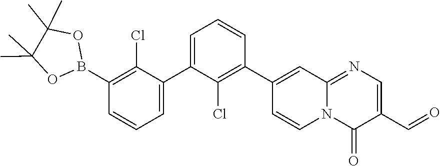 [SMILES: CC1(C)OB(c2cccc(-c3cccc(-c4ccn5c(=O)c(C=O)cnc5c4)c3Cl)c2Cl)OC1(C)C]

8-[3-(3-Bromo-2-chloro-phenyl)-2-chloro-phenyl]-4-oxo-pyrido[1,2-a]pyrimidine-3-carbaldehyde (0.89 g, 1.70 mmol), 4,4,5,5-tetramethyl-2-(4,4,5,5-tetramethyl-1,3,2-dioxaborolan-2-yl)-1,3,2-dioxaborae(0.56 g, 2.20 mmol), 1,1′-bis(diphenylphosphino)-ferrocene-palladium(ii) dichloride dichloromethane complex (0.14 g, 0.17 mmol) and potassium acetate (0.47 g, 4.75 mmol) were suspended in 15 ml anhydrous dioxane in a sealed tube. Nitrogen gas was bubbled through the reaction solution for 2 minutes, then the vessel was sealed and heated at 95° C. for 2 hours. The reaction was cooled to rt and the mixture was filtered through CELITE®. The filtrate was concentrated and the crude oil was purified by silica gel chromatography, eluting with 0-55% EtOAc gradient in hexane to afford 8-[2-chloro-3-[2-chloro-3-(4,4,5,5-tetramethyl-1,3,2-dioxaborolan-2-yl)phenyl]phenyl]-4-oxo-pyrido[1,2-a]pyrimidine-3-carbaldehyde (0.63 g, 71.2% yield) as a yellow solid. MS. m/z found 521, 523 [M+H]⁺.

### 8-(2,2′-Dichloro-3′-(5-formyl-6-methoxypyridin-2-yl)-[1,1′-biphenyl]-3-yl)-4-oxo-4H-pyrido[1,2-a]-pyrimidine-3-carbaldehyde

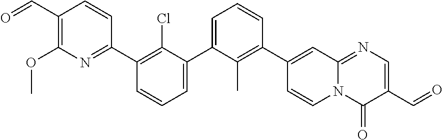 [SMILES: COc1nc(-c2cccc(-c3cccc(-c4ccn5c(=O)c(C=O)cnc5c4)c3C)c2Cl)ccc1C=O]

1,1′-Bis(diphenylphosphino)-ferrocene-palladium(ii) dichloride dichloromethane complex (20 mg, 0.02 mmol), potassium carbonate (86 mg, 0.62 mmol), 8-[2-chloro-3-[2-chloro-3-(4,4,5,5-tetramethyl-1,3,2-dioxaborolan-2-yl)phenyl]phenyl]-4-oxo-pyrido[1,2-a]pyrimidine-3-carbaldehyde (130 mg, 0.25 mmol) and 6-chloro-2-methoxy-pyridine-3-carbaldehyde (56 mg, 0.32 mmol) were suspended in 2 ml of 9:1 dioxane/water solvent mix, and the reaction was heated at 95° C. for 1 hour. The reaction was cooled to rt, filtered through CELITE®, and the filtrate was concentrated. The crude product was purified by silica gel chromatography, eluting with 0-60% EtOAc gradient in hexane to afford 8-[2-chloro-3-[2-chloro-3-(5-formyl-6-methoxy-2-pyridyl)phenyl]phenyl]-4-oxo-pyrido[1,2-a]pyrimidine-3-carbaldehyde (81 mg, 61.2% yield) as a yellow solid. MS: m/z found 530.1, 532.1 [M+H]⁺.

### N-(2-(((6-(3′-(3-(((2-Acetamidoethyl)amino)methyl)-4-oxo-4H-pyrido[1,2-a]pyrimidin-8-yl)-2,2′-dichloro-[1,1′-biphenyl]-3-yl)-2-methoxypyridin-3-yl)methyl)amino)ethyl)acetamide

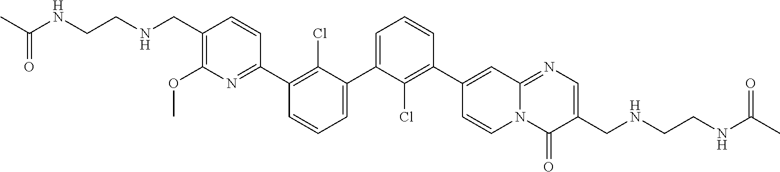 [SMILES: COc1nc(-c2cccc(-c3cccc(-c4ccn5c(=O)c(CNCCNC(C)=O)cnc5c4)c3Cl)c2Cl)ccc1CNCCNC(C)=O]

8-[2-Chloro-3-[2-chloro-3-(5-formyl-6-methoxy-2-pyridyl)phenyl]phenyl]-4-oxo-pyrido[1,2-a]pyrimidine-3-carbaldehyde (27 mg, 0.05 mmol), N-(2-aminoethyl)acetamide (16 mg, 0.15 mmol) and acetic acid (8 mg, 0.13 mmol) were dissolved in 1 ml of 1:1 MeOH/THF solvent mix, and the reaction was stirred at 25° C. for 12 hours. NaBH₃CN (10 mg, 0.15 mmol) was then added portion-wise, and the resulting mixture was stirred at 25° C. for 1 hour. The reaction was subsequently diluted with water (2 ml) and extracted with EtOAc (3×2 ml). The combined organics were further washed with brine, dried over sodium sulfate, then filtered and concentrated. Crude mixture was purified by reverse phase HPLC to afford N-[2-[[6-[3-[3-[3-[(2-acetamidoethylamino)methyl]-4-oxo-pyrido[1,2-a]pyrimidin-8-yl]-2-chloro-phenyl]-2-chloro-phenyl]-2-methoxy-3-pyridyl]methylamino]ethyl]acetamide (10.6 mg, 29.6% yield) as a clear oil. MS: m/z found 702.2, 704.2 [M+H]⁺. ¹H NMR (400 MHz, Chloroform-d) δ 9.08 (d, J=7.5 Hz, 1H), 8.33 (s, 1H), 7.80-7.73 (m, 1H), 7.69 (dd, J=7.7, 1.7 Hz, 1H), 7.59 (d, J=7.4 Hz, 1H), 7.53-7.42 (m, 4H), 7.38 (dd, J=7.4, 1.9 Hz, 1H), 7.33 (dd, J=7.6, 1.7 Hz, 1H), 7.28 (s, 1H), 6.33 (s, 1H), 6.07 (s, 1H), 4.03 (s, 3H), 3.85 (s, 2H), 3.80 (s, 2H), 3.39 (tt, J=8.2, 4.0 Hz, 4H), 2.80 (td, J=5.7, 1.9 Hz, 4H), 2.00 (d, J=3.7 Hz, 6H).

### Example 70: 8-(2,2′-Dichloro-3′-(6-methoxy-5-((7-oxo-2,6-diazaspiro[3.4]octan-2-yl)methyl)pyridin-2-yl)-[1,1′-biphenyl]-3-yl)-3-((7-oxo-2,6-diazaspiro[3.4]octan-2-yl)methyl)-4H-pyrido[1,2-a]pyrimidin-4-one

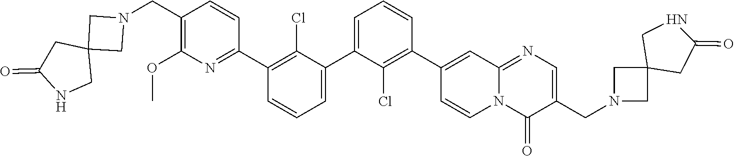 [SMILES: COc1nc(-c2cccc(-c3cccc(-c4ccn5c(=O)c(CN6CC7(CNC(=O)C7)C6)cnc5c4)c3Cl)c2Cl)ccc1CN1CC2(CNC(=O)C2)C1]

8-[2-Chloro-3-[2-chloro-3-(5-formyl-6-methoxy-2-pyridyl)phenyl]phenyl]-4-oxo-pyrido[1,2-a]pyrimidine-3-carbaldehyde (27 mg, 0.05 mmol), 2,6-diazaspiro[3.4]octan-7-one; 4-methylbenzenesulfonic acid (46 mg, 0.15 mmol) and DIEA (44 μL, 33 mg, 0.25 mmol) were dissolved in 1 ml of 1:1 MeOH/THF solvent mix, and the reaction was stirred at 25° C. for 12 hours. NaBH₃CN (10 mg, 0.15 mmol) and acetic acid (8 mg, 0.13 mmol) were then added, and the resulting mixture was stirred at 25° C. for 1 hour. The reaction was subsequently diluted with water (2 mL) and extracted with EtOAc (3×2 mL). The combined organics were further washed with brine, dried over sodium sulfate, then filtered and concentrated. Crude mixture was purified by reverse phase HPLC to afford 8-[2-chloro-3-[2-chloro-3-[6-methoxy-5-[(6-oxo-2,7-diazaspiro[3.4]octan-2-yl)methyl]-2-pyridyl]phenyl]phenyl]-3-[(6-oxo-2,7-diazaspiro[3.4]octan-2-yl)methyl]pyrido[1,2-a]pyrimidin-4-one (11.6 mg, 30.4% yield) as a clear oil. MS: m/z found 750.3, 752.3 [M+H]⁺. ¹H NMR (400 MHz, Chloroform-d) δ9.08 (d, J=7.4 Hz, 1H), 8.35 (s, 1H), 7.74 (d, J=1.8 Hz, 1H), 7.68 (dd, J=7.6, 1.6 Hz, 1H), 7.62 (d, J=7.5 Hz, 1H), 7.52-7.41 (m, 4H), 7.34 (ddd, J=15.4, 7.4, 1.8 Hz, 2H), 7.28 (s, 1H), 5.75 (d, J=12.3 Hz, 2H), 4.00 (d, J=1.1 Hz, 3H), 3.70 (s, 2H), 3.65 (s, 2H), 3.60 (d, J=6.5 Hz, 4H), 3.39 (d, J=7.2 Hz, 4H), 3.33 (dd, J=7.3, 4.6 Hz, 4H), 2.55 (d, J=3.9 Hz, 4H).

### Example 71: (S)-8-(3′-(5-(3-Aminoazetidin-1-yl)-6-methoxypyrazin-2-yl)-2,2′-dichloro-[1,1′-biphenyl]-3-yl)-3-((((5-oxopyrrolidin-2-yl)methyl)amino)methyl)-4H-pyrido[1,2-a]pyrimidin-4-one

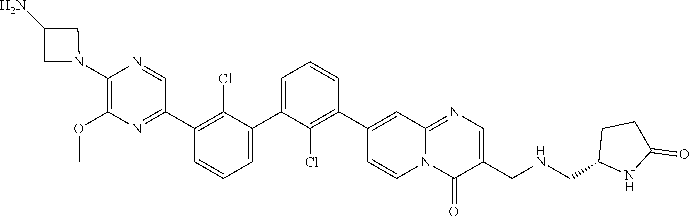 [SMILES: COc1nc(-c2cccc(-c3cccc(-c4ccn5c(=O)c(CNC[C@@H]6CCC(=O)N6)cnc5c4)c3Cl)c2Cl)cnc1N1CC(N)C1]

### tert-Butyl (1-(5-bromo-3-methoxypyrazin-2-yl)-azetidin-3-yl)-carbamate

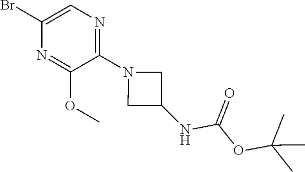 [SMILES: COc1nc(Br)cnc1N1CC(NC(=O)OC(C)(C)C)C1]

5-Bromo-2-iodo-3-methoxy-pyrazine (75 mg, 0.24 mmol) and tert-butyl N-(azetidin-3-yl)-carbamate (123 mg, 0.71 mmol) were heat neat at 100° C. for 30 minutes. The crude residue was suspended in 2 mL DCM and the insoluble un-reacted amine was filtered off. The filtrate was concentrated and purified by silica gel chromatography, eluting with 0-15% EtOAc gradient in hexane to afford tert-butyl N-[1-(5-bromo-3-methoxy-pyrazin-2-yl)-azetidin-3-yl]carbamate (45 mg, 52.6% yield) as a white solid. MS: m/z found 359, 361 [M+H]⁺.

### tert-Butyl (1-(5-(2,2′-dichloro-3′-(3-formyl-4-oxo-4H-pyrido[1,2-a]pyrimidin-8-yl)-[1,1′-biphenyl]-3-yl)-3-methoxypyrazin-2-yl)azetidin-3-yl)carbamate

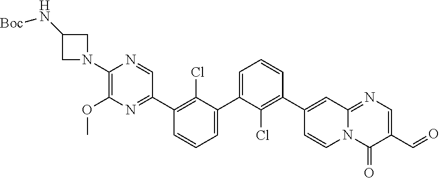 [SMILES: COc1nc(-c2cccc(-c3cccc(-c4ccn5c(=O)c(C=O)cnc5c4)c3Cl)c2Cl)cnc1N1CC(NC(=O)OC(C)(C)C)C1]

1,1′-Bis(diphenylphosphino)-ferrocene-palladium(ii) dichloride dichloromethane complex (6 mg, 0.01 mmol), potassium carbonate (26 mg, 0.19 mmol), 8-[2-chloro-3-[2-chloro-3-(4,4,5,5-tetramethyl-1,3,2-dioxaborolan-2-yl)phenyl]phenyl]-4-oxo-pyrido[1,2-a]pyrimidine-3-carbaldehyde (40 mg, 0.08 mmol) and tert-butyl N-[1-(5-bromo-3-methoxy-pyrazin-2-yl)azetidin-3-yl]carbamate (36 mg, 0.10 mmol) were suspended in 2 mL of 9:1 dioxane/water solvent mixture, and the reaction was heated at 95° C. for 1 hour. The reaction was cooled to rt, filtered through CELITE®, and the filtrate was concentrated. Crude product was purified by silica gel chromatography, eluting with 0-65% EtOAc gradient in hexane to afford tert-butyl N-[1-[5-[2-chloro-3-[2-chloro-3-(3-formyl-4-oxo-pyrido[1,2-a]pyrimidin-8-yl)phenyl]phenyl]-3-methoxy-pyrazin-2-yl]azetidin-3-yl]carbamate (15.6 mg, 30.2% yield) as a yellow solid. MS: m/z found 673.2, 675.2 [M+H]⁺.

### tert-Butyl (S)-(1-(5-(2,2′-dichloro-3′-(4-oxo-3-((((5-oxopyrrolidin-2-yl)methyl)amino)methyl)-4H-pyrido[1,2-a]pyrimidin-8-yl)-[1,1′-biphenyl]-3-yl)-3-methoxypyrazin-2-yl)azetidin-3-yl)carbamate

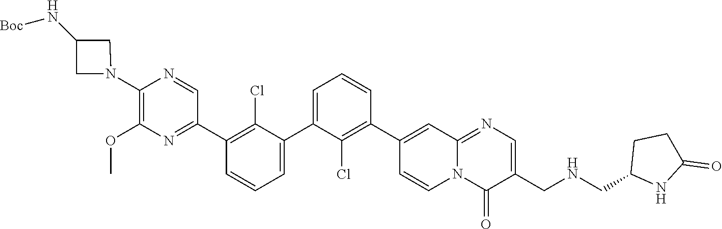 [SMILES: COc1nc(-c2cccc(-c3cccc(-c4ccn5c(=O)c(CNC[C@@H]6CCC(=O)N6)cnc5c4)c3Cl)c2Cl)cnc1N1CC(NC(=O)OC(C)(C)C)C1]

tert-Butyl N-[1-[5-[2-chloro-3-[2-chloro-3-(3-formyl-4-oxo-pyrido[1,2-a]pyrimidin-8-yl)phenyl]phenyl]-3-methoxy-pyrazin-2-yl]azetidin-3-yl]carbamate (16 mg, 0.02 mmol), (5S)-5-(aminomethyl)pyrrolidin-2-one (3 mg, 0.03 mmol) and acetic acid (2 mg, 0.03 mmol) were dissolved in 1 mL of 1:1 MeOH/THF solvent mix, and the reaction was stirred at 25° C. for 3 hours. NaBH₃CN, (2 mg, 0.03 mmol) was then added portion-wise, and the resulting mixture was stirred at 25° C. for 1 hour. The reaction was subsequently diluted with water (5 ml) and extracted with EtOAc (3×3 mL). The combined organics were further washed with brine, dried over sodium sulfate, then filtered and concentrated to afford tert-butyl N-[1-[5-[2-chloro-3-[2-chloro-3-[4-oxo-3-[[[(2S)-5-oxopyrrolidin-2-yl]methylamino]methyl]pyrido[1,2-a]pyrimidin-8-yl]phenyl]phenyl]-3-methoxy-pyrazin-2-yl]azetidin-3-yl]carbamate (17.9 mg) as a white solid. Material was used as—is for the next step. MS: m/z found 771, 773 [M+H]⁺.

### (S)-8-(3′-(5-(3-Aminoazetidin-1-yl)-6-methoxypyrazin-2-yl)-2,2′-dichloro-[1,1′-biphenyl]-3-yl)-3-((((5-oxopyrrolidin-2-yl)methyl)amino)methyl)-4H-pyrido[1,2-a]pyrimidin-4-one

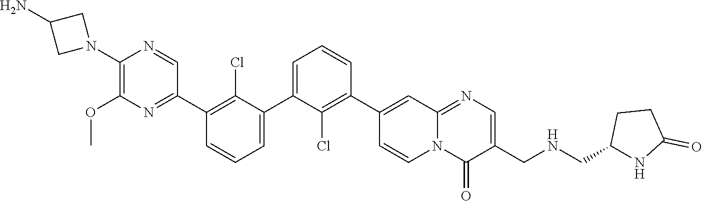 [SMILES: COc1nc(-c2cccc(-c3cccc(-c4ccn5c(=O)c(CNC[C@@H]6CCC(=O)N6)cnc5c4)c3Cl)c2Cl)cnc1N1CC(N)C1]

tert-Butyl N-[1-[5-[2-chloro-3-[2-chloro-3-[4-oxo-3-[[[(2S)-5-oxopyrrolidin-2-yl]methylamino]methyl]pyrido[1,2-a]pyrimidin-8-yl]phenyl]phenyl]-3-methoxy-pyrazin-2-yl]azetidin-3-yl]carbamate (18 mg, 0.02 mmol) was dissolved in 0.5 mL DCM and 4M HCl solution in dioxane (29 μL, 4 mg, 0.12 mmol) was added. The reaction was stirred at rt for 20 minutes and the precipitate was filtered and further washed with DCM (2×3 mL). The resulting solid was dissolved in 1 mL water and the pH was adjusted to ˜ pH 9 by slow addition of aqueous saturated sodium bicarbonate solution. The aqueous was extracted with DCM (3×1 ml) and the combined organics were washed with brine, dried over sodium sulfate, filtered and concentrated to afford 8-[3-[3-[5-(3-aminoazetidin-1-yl)-6-methoxy-pyrazin-2-yl]-2-chloro-phenyl]-2-chloro-phenyl]-3-[[[(2S)-5-oxopyrrolidin-2-yl]methylamino]methyl]pyrido[1,2-a]pyrimidin-4-one (3.1 mg, 19.9% yield) as a clear film.

MS: m/z found 671.75, 673 [M+H]⁺. ¹H NMR (400 MHz, Chloroform-d) δ 9.08 (d, J=7.5 Hz, 1H), 8.32 (s, 1H), 8.05 (s, 1H), 7.76 (d, J=1.9 Hz, 1H), 7.66 (dd, J=7.7, 1.7 Hz, 1H), 7.52-7.36 (m, 5H), 7.28 (dd, J=7.5, 1.6 Hz, 1H), 6.10 (s, 1H), 4.50 (dd, J=9.3, 6.9 Hz, 2H), 3.98 (s, 4H), 3.91-3.84 (m, 4H), 3.78 (s, 1H), 2.81 (dd, J=11.9, 4.2 Hz, 1H), 2.56 (dd, J=12.0, 8.8 Hz, 1H), 2.40-2.31 (m, 2H), 2.29-2.17 (m, 1H), 1.79-1.71 (m, 1H).

### Example 72: 8-(2′-Chloro-3′-(6-ethoxy-5-(((((S)-5-oxopyrrolidin-2-yl)methyl)amino)methyl)pyridin-2-yl)-2-methyl-[1,1′-biphenyl]-3-yl)-3-(((((S)-5-oxopyrrolidin-2-yl)methyl)amino)methyl)-4H-pyrido[1,2-a]pyrimidin-4-one

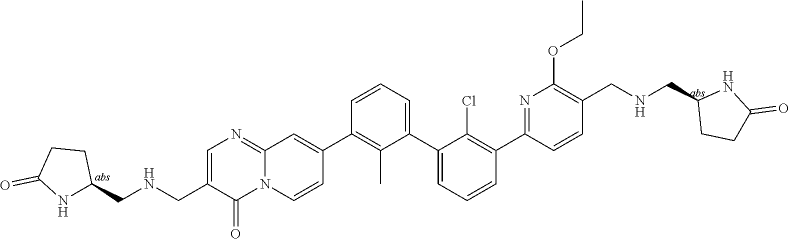 [SMILES: CCOc1nc(-c2cccc(-c3cccc(-c4ccn5c(=O)c(CNC[C@@H]6CCC(=O)N6)cnc5c4)c3C)c2Cl)ccc1CNC[C@@H]1CCC(=O)N1]

### 6-Bromo-2-ethoxy-pyridine-3-carbaldehyde

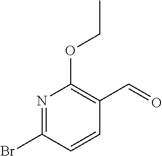

To a solution of 6-bromo-2-hydroxy-pyridine-3-carbaldehyde (90 mg, 0.45 mmol) in DMF was added potassium carbonate (184 mg, 1.34 mmol) and the mixture was stirred for 10 minutes. Ethyl iodide (107 uL, 208 mg, 1.34 mmol) was added and the reaction mixture was stirred at 55° C. overnight. After workup, the crude mixture was purified on silica gel column using EtOAc in hexanes (0 to 40% gradient) to give 6-bromo-2-ethoxy-pyridine-3-carbaldehyde (42 mg, 41.0%). ¹H NMR (400 MHz, Chloroform-d): δ 10.32 (d, J=0.8 Hz, 1H), 7.93 (dd, J=7.8, 0.7 Hz, 1H), 7.17 (dt, J=7.8, 0.7 Hz, 1H), 4.58-4.48 (m, 2H), 1.45 (td, J=7.1, 0.7 Hz, 3H).

### 8-[3-[2-Chloro-3-(6-ethoxy-5-formyl-2-pyridyl)phenyl]-2-methyl-phenyl]-4-oxo-pyrido[1,2-a]pyrimidine-3-carbaldehyde

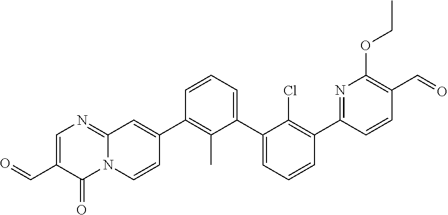 [SMILES: CCOc1nc(-c2cccc(-c3cccc(-c4ccn5c(=O)c(C=O)cnc5c4)c3C)c2Cl)ccc1C=O]

To a solution of 8-[3-[2-chloro-3-(4,4,5,5-tetramethyl-1,3,2-dioxaborolan-2-yl)phenyl]-2-methyl-phenyl]-4-oxo-pyrido[1,2-a]pyrimidine-3-carbaldehyde (70 mg, 0.14 mmol), 6-bromo-2-ethoxy-pyridine-3-carbaldehyde (39 mg, 0.17 mmol) and potassium carbonate (58 mg, 0.42 mmol) in degassed dioxane/water (5:1) was added Pd(PPh₃)₄ (16 mg, 0.01 mmol) and the mixture was stirred at 90° C. for 3 hours under N₂(g). Water was added and the mixture was extracted into EtOAc three times. The combined organic layers were dried over anhydrous sodium sulfate, concentrated, and purified on silica gel column using EtOAc in hexanes (0 to 60% gradient) to yield 8-[3-[2-chloro-3-(6-ethoxy-5-formyl-2-pyridyl)phenyl]-2-methyl-phenyl]-4-oxo-pyrido[1,2-a]pyrimidine-3-carbaldehyde (45 mg, 61.4%). MS: m/z found 524.2 [M+H]⁺.

### 8-[3-[2-Chloro-3-[6-ethoxy-5-[[[(2S)-5-oxopyrrolidin-2-yl]methylamino]methyl]-2-pyridyl]phenyl]-2-methyl-phenyl]-3-[[[(2S)-5-oxopyrrolidin-2-yl]methylamino]methyl]pyrido[1,2-a]pyrimidin-4-one

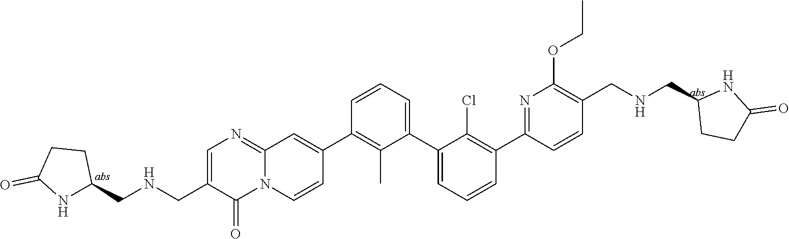 [SMILES: CCOc1nc(-c2cccc(-c3cccc(-c4ccn5c(=O)c(CNC[C@@H]6CCC(=O)N6)cnc5c4)c3C)c2Cl)ccc1CNC[C@@H]1CCC(=O)N1]

A mixture of 8-[3-[2-chloro-3-(6-ethoxy-5-formyl-2-pyridyl)phenyl]-2-methyl-phenyl]-4-oxo-pyrido[1,2-a]pyrimidine-3-carbaldehyde (40 mg, 0.08 mmol), (5S)-5-(aminomethyl)pyrrolidin-2-one (35 mg, 0.31 mmol) and acetic acid (9 uL, 9 mg, 0.15 mmol) in MeOH/THF (1:1) was stirred for 1 hour at rt. NaBH₃CN (19 mg, 0.31 mmol) was added, the mixture was stirred for additional 9 h and then quenched with water and directly purified by HPLC to yield 8-[3-[2-chloro-3-[6-ethoxy-5-[[[(2S)-5-oxopyrrolidin-2-yl]methylamino]methyl]-2-pyridyl]phenyl]-2-methyl-phenyl]-3-[[[(2S)-5-oxopyrrolidin-2-yl]methylamino]methyl]pyrido[1,2-a]pyrimidin-4-one (45 mg, 81.8%). MS: m/z found 720.5 [M+H]⁺; ¹H NMR (400 MHz, Methanol-d₄): δ 9.10 (dq, J=7.4, 0.9 Hz, 1H), 8.39 (s, 1H), 7.75-7.68 (m, 1H), 7.65-7.61 (m, 1H), 7.59 (dd, J=7.7, 1.8 Hz, 1H), 7.48 (t, J=7.6 Hz, 1H), 7.42 (ddd, J=5.3, 4.3, 2.2 Hz, 3H), 7.34 (dd, J=7.5, 1.7 Hz, 1H), 7.32-7.29 (m, 1H), 7.20 (d, J=7.4 Hz, 1H), 4.46 (q, J=7.0 Hz, 2H), 3.90-3.78 (m, 6H), 2.79-2.63 (m, 4H), 2.38-2.20 (m, 6H), 2.11 (s, 3H), 1.88-1.74 (m, 2H), 1.41 (t, J=7.0 Hz, 3H).

### Example 73: 8-(2′-Chloro-2-methyl-3′-(6-(methylamino)-5-(((((S)-5-oxopyrrolidin-2-yl)methyl)amino)methyl)pyridin-2-yl)-[1,1′-biphenyl]-3-yl)-3-(((((S)-5-oxopyrrolidin-2-yl)methyl)amino)methyl)-4H-pyrido[1,2-a]pyrimidin-4-one

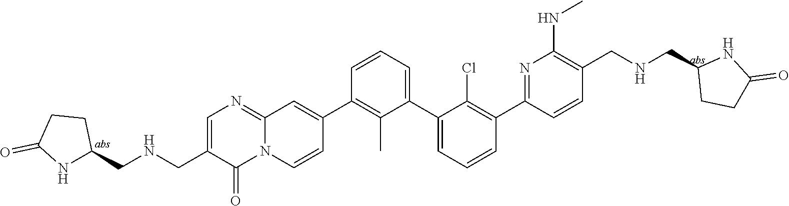 [SMILES: CNc1nc(-c2cccc(-c3cccc(-c4ccn5c(=O)c(CNC[C@@H]6CCC(=O)N6)cnc5c4)c3C)c2Cl)ccc1CNC[C@@H]1CCC(=O)N1]

### 6-Bromo-2-(methylamino)pyridine-3-carbaldehyde

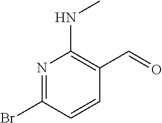

To a solution of 6-bromo-2-fluoro-pyridine-3-carbaldehyde (100 mg, 0.49 mmol) in DMSO were added methylamine hydrochloride (66 mg, 0.98 mmol) and potassium carbonate (142 mg, 1.03 mmol). The resulting mixture was stirred overnight at 70° C., cooled to rt, and purified on silica gel column using EtOAc in hexanes (0 to 20% gradient) to give 6-bromo-2-(methylamino) pyridine-3-carbaldehyde (90 mg, 85.4%). MS: m/z found 215.1 [M+H]⁺; ¹H NMR (400 MHz, Chloroform-d): δ9.75 (d, J=0.5 Hz, 1H), 8.46 (br s, 1H), 7.51 (dd, J=7.8, 0.5 Hz, 1H), 6.77 (dd, J=7.8, 0.5 Hz, 1H), 3.08 (dd, J=5.0, 0.5 Hz, 3H).

### 8-[3-[2-Chloro-3-[5-formyl-6-(methylamino)-2-pyridyl]phenyl]-2-methyl-phenyl]-4-oxo-pyrido[1,2-a]pyrimidine-3-carbaldehyde

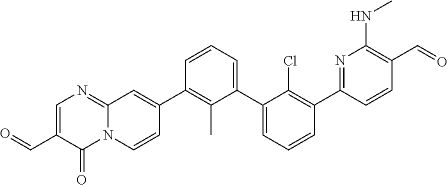 [SMILES: CNc1nc(-c2cccc(-c3cccc(-c4ccn5c(=O)c(C=O)cnc5c4)c3C)c2Cl)ccc1C=O]

To a solution of 8-[3-[2-chloro-3-(4,4,5,5-tetramethyl-1,3,2-dioxaborolan-2-yl)phenyl]-2-methyl-phenyl]-4-oxo-pyrido[1,2-a]pyrimidine-3-carbaldehyde (65 mg, 0.13 mmol), 6-bromo-2-(methylamino)pyridine-3-carbaldehyde (34 mg, 0.16 mmol) and potassium carbonate (54 mg, 0.39 mmol) in degassed dioxane/water (5:1) was added Pd(PPh₃)₄ (15 mg, 0.01 mmol) and the mixture was stirred at 90° C. for 3 hours under N₂(g). Water was added and the mixture was extracted into EtOAc three times. The combined organic layers were dried over anhydrous sodium sulfate, concentrated, and purified on silica gel column using EtOAc in hexanes (0 to 60% gradient). MS: m/z found 509.1 [M+H]⁺.

### 8-[3-[2-Chloro-3-[6-(methylamino)-5-[[[(2S)-5-oxopyrrolidin-2-yl]methylamino]methyl]-2-pyridyl]phenyl]-2-methyl-phenyl]-3-[[[(2S)-5-oxopyrrolidin-2-yl]methylamino]methyl]pyrido[1,2-a]pyrimidin-4-one

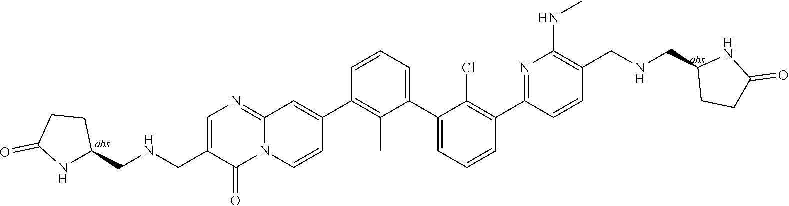 [SMILES: CNc1nc(-c2cccc(-c3cccc(-c4ccn5c(=O)c(CNC[C@@H]6CCC(=O)N6)cnc5c4)c3C)c2Cl)ccc1CNC[C@@H]1CCC(=O)N1]

A mixture of 8-[3-[2-chloro-3-[5-formyl-6-(methylamino)-2-pyridyl]phenyl]-2-methyl-phenyl]-4-oxo-pyrido[1,2-a]pyrimidine-3-carbaldehyde (35 mg, 0.07 mmol), (5S)-5-(aminomethyl)pyrrolidin-2-one (31 mg, 0.28 mmol) and acetic acid (8 uL, 8 mg, 0.14 mmol) in MeOH/THF (1:1) was stirred for 2 hour at rt. NaBH₃CN (17 mg, 0.28 mmol) was added, the mixture was stirred for additional 5 h, quenched with water and directly purified by HPLC. MS: m/z found 705.4 [M+H]⁺. The collected fraction was neutralized with sat. NaHCO₃(aq.), acetonitrile was evaporated, and aqueous phase was extracted into DCM and EtOAc three times. The combined organic extracts were dried over anhydrous magnesium sulfate and concentrated in vacuo to give 8-[3-[2-chloro-3-[6-(methylamino)-5-[[[(2S)-5-oxopyrrolidin-2-yl]methylamino]methyl]-2-pyridyl]phenyl]-2-methyl-phenyl]-3-[[[(2S)-5-oxopyrrolidin-2-yl]methylamino]methyl]pyrido[1,2-a]pyrimidin-4-one (34 mg, 70.1%). ¹H NMR (400 MHz, Methanol-d₄): δ 9.09 (dd, J=7.3, 0.8 Hz, 1H), 8.39 (d, J=0.6 Hz, 1H), 7.62 (dd, J=1.9, 0.8 Hz, 1H), 7.56 (ddd, J=7.7, 1.8, 0.6 Hz, 1H), 7.47-7.41 (m, 2H), 7.41-7.35 (m, 3H), 7.32-7.27 (m, 2H), 6.73 (d, J=7.3 Hz, 1H), 3.91-3.65 (m, 6H), 2.97 (s, 3H), 2.78-2.64 (m, 4H), 2.37-2.19 (m, 6H), 2.11 (s, 3H), 1.87-1.75 (m, 2H).

### Example 74: 8-(2′-Chloro-3′-(6-(ethylamino)-5-(((((S)-5-oxopyrrolidin-2-yl)methyl)amino)methyl)pyridin-2-yl)-2-methyl-[1,1′-biphenyl]-3-yl)-3-(((((S)-5-oxopyrrolidin-2-yl)methyl)amino)methyl)-4H-pyrido[1,2-a]pyrimidin-4-one

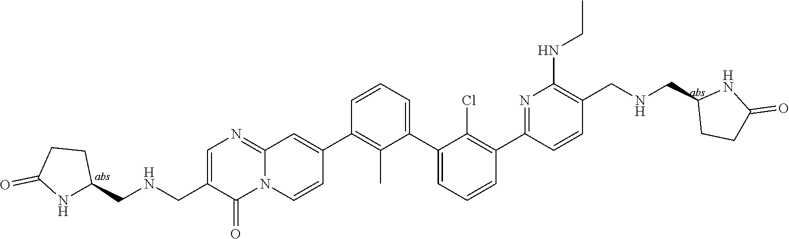 [SMILES: CCNc1nc(-c2cccc(-c3cccc(-c4ccn5c(=O)c(CNC[C@@H]6CCC(=O)N6)cnc5c4)c3C)c2Cl)ccc1CNC[C@@H]1CCC(=O)N1]

### 6-Bromo-2-(ethylamino)pyridine-3-carbaldehyde

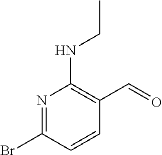

To a solution of 6-bromo-2-fluoro-pyridine-3-carbaldehyde (100 mg, 0.49 mmol) in THE was added ethanamine (44 mg, 0.98 mmol). 2M solution of ethanamine in THE was used. The resulting mixture was refluxed overnight, cooled to rt, and purified on silica gel column using EtOAc in hexanes (0 to 20% gradient) to give 6-bromo-2-(ethylamino)pyridine-3-carbaldehyde (90 mg, 80.1%). MS: m/z found 229.1 [M+H]⁺; ¹H NMR (400 MHz, Chloroform-d): δ 9.74 (s, 1H), 8.46 (br s, 1H), 7.50 (d, J=7.8 Hz, 1H), 6.75 (d, J=7.8 Hz, 1H), 3.56 (qd, J=7.3, 5.5 Hz, 2H), 1.26 (t, J=7.3 Hz, 3H).

### 8-[3-[2-Chloro-3-[6-(ethylamino)-5-formyl-2-pyridyl]phenyl]-2-methyl-phenyl]-4-oxo-pyrido[1,2-a]pyrimidine-3-carbaldehyde

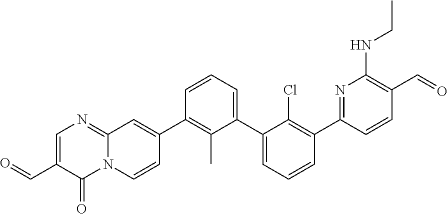 [SMILES: CCNc1nc(-c2cccc(-c3cccc(-c4ccn5c(=O)c(C=O)cnc5c4)c3C)c2Cl)ccc1C=O]

To a solution of 8-[3-[2-chloro-3-(4,4,5,5-tetramethyl-1,3,2-dioxaborolan-2-yl)phenyl]-2-methyl-phenyl]-4-oxo-pyrido[1,2-a]pyrimidine-3-carbaldehyde (65 mg, 0.13 mmol), 6-bromo-2-(ethylamino)pyridine-3-carbaldehyde (36 mg, 0.16 mmol) and potassium carbonate (54 mg, 0.39 mmol) in degassed dioxane/water (5:1) was added Pd(PPh₃)₄ (15 mg, 0.01 mmol) and the mixture was stirred at 90° C. for overnight under N₂(g). Water was added and the mixture was extracted into EtOAc three times. The combined organic layers were dried over anhydrous sodium sulfate, concentrated, and purified on silica gel column using EtOAc in hexanes (0 to 60% gradient) to give 8-[3-[2-chloro-3-[6-(ethylamino)-5-formyl-2-pyridyl]phenyl]-2-methyl-phenyl]-4-oxo-pyrido[1,2-a]pyrimidine-3-carbaldehyde (40 mg, 58.9%). MS: m/z found 523.3 [M+H]⁺.

### 8-[3-[2-Chloro-3-[6-(ethylamino)-5-[[[(2S)-5-oxopyrrolidin-2-yl]methylamino]methyl]-2-pyridyl]phenyl]-2-methyl-phenyl]-3-[[[(2S)-5-oxopyrrolidin-2-yl]methylamino]methyl]pyrido[1,2-a]pyrimidin-4-one

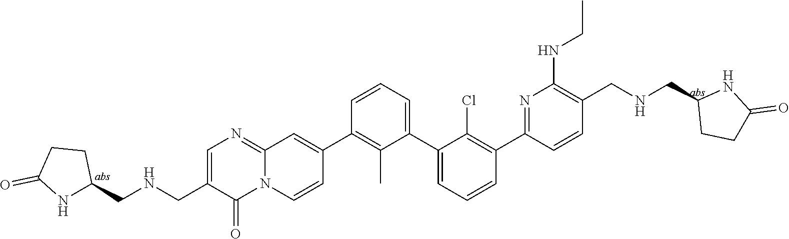 [SMILES: CCNc1nc(-c2cccc(-c3cccc(-c4ccn5c(=O)c(CNC[C@@H]6CCC(=O)N6)cnc5c4)c3C)c2Cl)ccc1CNC[C@@H]1CCC(=O)N1]

A mixture of 8-[3-[2-chloro-3-[6-(ethylamino)-5-formyl-2-pyridyl]phenyl]-2-methyl-phenyl]-4-oxo-pyrido[1,2-a]pyrimidine-3-carbaldehyde (40 mg, 0.08 mmol), (5S)-5-(aminomethyl)pyrrolidin-2-one (36 mg, 0.31 mmol) and acetic acid (9 uL, 9 mg, 0.16 mmol) in MeOH/THF (1:1) was stirred for 1 hour at rt. NaBH₃CN (20 mg, 0.31 mmol) was added, the mixture was stirred for additional 6 h, quenched with water and directly purified by HPLC. MS: m/z found 719.3 [M+H]⁺. The collected fraction was neutralized with sat. NaHCO₃(aq.), acetonitrile was evaporated, and aqueous phase was extracted into DCM and EtOAc three times. The combined organic extracts were dried over anhydrous magnesium sulfate and concentrated in vacuo to give 8-[3-[2-chloro-3-[6-(ethylamino)-5-[[[(2S)-5-oxopyrrolidin-2-yl]methylamino]methyl]-2-pyridyl]phenyl]-2-methyl-phenyl]-3-[[[(2S)-5-oxopyrrolidin-2-yl]methylamino]methyl]pyrido[1,2-a]pyrimidin-4-one (38 mg, 67.2%). ¹H NMR (400 MHz, Methanol-d₄): δ 9.11 (dd, J=7.3, 0.7 Hz, 1H), 8.39 (s, 1H), 7.63 (dq, J=1.7, 0.9 Hz, 1H), 7.53 (ddd, J=10.9, 7.6, 1.8 Hz, 1H), 7.47-7.38 (m, 4H), 7.36 (dd, J=7.5, 1.7 Hz, 1H), 7.33-7.28 (m, 2H), 6.73 (d, J=7.3 Hz, 1H), 3.91-3.71 (m, 6H), 3.46 (qd, J=7.2, 1.5 Hz, 2H), 2.78-2.64 (m, 4H), 2.41-2.20 (m, 6H), 2.12 (s, 3H), 1.90-1.75 (m, 2H), 1.25 (t, J=7.2 Hz, 3H).

### Example 75: 8-(2,2′-Dichloro-3′-(5-(((2-hydroxyethyl)amino)methyl)-6-methoxypyridin-2-yl)-[1,1′-biphenyl]-3-yl)-3-(((2-hydroxyethyl)amino)methyl)-4H-pyrido[1,2-a]pyrimidin-4-one

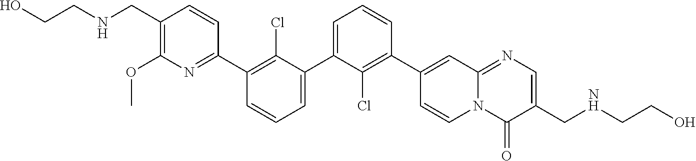 [SMILES: COc1nc(-c2cccc(-c3cccc(-c4ccn5c(=O)c(CNCCO)cnc5c4)c3Cl)c2Cl)ccc1CNCCO]

8-[2-Chloro-3-[2-chloro-3-(5-formyl-6-methoxy-2-pyridyl)phenyl]phenyl]-4-oxo-pyrido[1,2-a]pyrimidine-3-carbaldehyde (27 mg, 0.05 mmol), 2-aminoethanol (12 mg, 0.20 mmol) and acetic acid (9 mg, 0.15 mmol) were dissolved in 1 mL of 1:1 MeOH/THF solvent mix, and the reaction was stirred at 25° C. for 3 hours. NaBH₃CN (9.6 mg, 0.15 mmol) was then added portion-wise, and the resulting mixture was stirred at 25° C. for 1 hour. The reaction was subsequently diluted with water (2 ml) and extracted with EtOAc (3×2 mL). The combined organics were further washed with brine, dried over sodium sulfate, then filtered and concentrated. The crude mixture was purified by reverse phase HPLC to afford 8-[2-chloro-3-[2-chloro-3-[5-[(2-hydroxyethylamino)methyl]-6-methoxy-2-pyridyl]phenyl]phenyl]-3-[(2-hydroxyethylamino)methyl]pyrido[1,2-a]pyrimidin-4-one (11 mg, 34.8% yield) as an off-white foam. MS: m/z found 620.3, 622.3 [M+H]⁺. ¹H NMR (400 MHz, Chloroform-d) δ 9.08 (d, J=7.3 Hz, 1H), 8.36 (s, 1H), 7.76 (d, J=1.9 Hz, 1H), 7.68 (dt, J=7.8, 1.6 Hz, 1H), 7.62 (d, J=7.4 Hz, 1H), 7.52-7.42 (m, 4H), 7.38 (dt, J=7.2, 1.7 Hz, 1H), 7.33 (dd, J=7.5, 1.7 Hz, 1H), 7.28 (s, 1H), 4.03 (d, J=1.2 Hz, 3H), 3.91 (s, 2H), 3.86 (s, 2H), 3.71 (t, J=5.3 Hz, 4H), 2.85 (t, J=5.2 Hz, 4H).

### Example 76: 8-(2′-Chloro-2-methyl-3′-(1-((((S)-5-oxopyrrolidin-2-yl)methyl)amino)-2,3-dihydro-1H-inden-5-yl)-[1,1′-biphenyl]-3-yl)-3-(((((S)-5-oxopyrrolidin-2-yl)methyl)amino)methyl)-4H-pyrido[1,2-a]pyrimidin-4-one

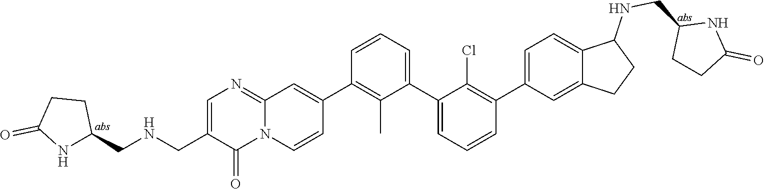 [SMILES: Cc1c(-c2ccn3c(=O)c(CNC[C@@H]4CCC(=O)N4)cnc3c2)cccc1-c1cccc(-c2ccc3c(c2)CCC3NC[C@@H]2CCC(=O)N2)c1Cl]

### (5S)-5-[[(5-Bromoindan-1-yl)amino]methyl]pyrrolidin-2-one

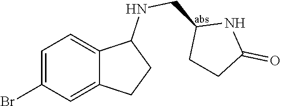 [SMILES: O=C1CC[C@@H](CNC2CCc3cc(Br)ccc32)N1]

To a solution of 5-bromoindan-1-one (200 mg, 0.95 mmol) and (5S)-5-(aminomethyl)pyrrolidin-2-one (216 mg, 1.90 mmol) in MeOH/THF (1:1) were added acetic acid (54 uL, 7 mg, 0.95 mmol) and NaBH₃CN (119 mg, 1.90 mmol). The resulting mixture was stirred at rt overnight and concentrated. Water was added and the residue was extracted into EtOAc and DCM three times. The combined organic layers were dried, concentrated, and used in the next step without further purification. MS: m/z found 309.1 [M+H]⁺.

### tert-Butyl N-(5-bromoindan-1-yl)-N-[[(2S)-5-oxopyrrolidin-2-yl]methyl]carbamate

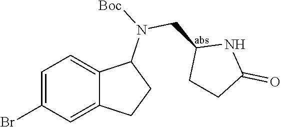 [SMILES: CC(C)(C)OC(=O)N(C[C@@H]1CCC(=O)N1)C1CCc2cc(Br)ccc21]

A mixture of (5S)-5-[[(5-bromoindan-1-yl)amino]methyl]pyrrolidin-2-one (200 mg, 0.65 mmol) and di-tert-butyl dicarbonate (184 mg, 0.84 mmol) in acetonitrile was stirred at rt overnight, concentrated and purified on silica gel column (MeOH in DCM, 0 to 10% gradient) to provide tert-butyl N-(5-bromoindan-1-yl)-N-[[(2S)-5-oxopyrrolidin-2-yl]methyl]carbamate (230 mg, 86.9%). MS: m/z found 409.2 [M+H]⁺.

### tert-Butyl N-[5-[2-chloro-3-[3-(3-formyl-4-oxo-pyrido[1,2-a]pyrimidin-8-yl)-2-methyl-phenyl]phenyl]indan-1-yl]-N-[[(2S)-5-oxopyrrolidin-2-yl]methyl]carbamate

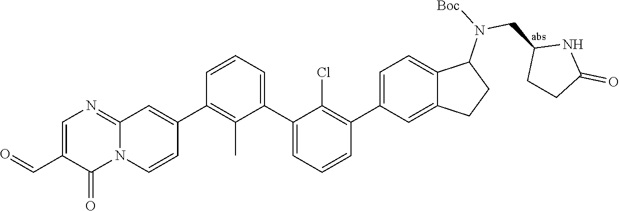 [SMILES: Cc1c(-c2ccn3c(=O)c(C=O)cnc3c2)cccc1-c1cccc(-c2ccc3c(c2)CCC3N(C[C@@H]2CCC(=O)N2)C(=O)OC(C)(C)C)c1Cl]

To a solution of 8-[3-[2-chloro-3-(4,4,5,5-tetramethyl-1,3,2-dioxaborolan-2-yl)phenyl]-2-methyl-phenyl]-4-oxo-pyrido[1,2-a]pyrimidine-3-carbaldehyde (60 mg, 0.12 mmol), tert-butyl N-(5-bromoindan-1-yl)-N-[[(2S)-5-oxopyrrolidin-2-yl]methyl]carbamate (59 mg, 0.14 mmol) and potassium carbonate (50 mg, 0.36 mmol) in degassed dioxane/water (5:1) was added Pd(PPh₃)₄ (14 mg, 0.01 mmol) and the mixture was stirred at 90° C. for 3 hours under N₂(g). Water was added and the mixture was extracted into EtOAc three times. The combined organic layers were dried over anhydrous sodium sulfate, concentrated, and purified on silica gel column using MeOH in DCM (0 to 5% gradient) to give tert-butyl N-[5-[2-chloro-3-[3-(3-formyl-4-oxo-pyrido[1,2-a]pyrimidin-8-yl)-2-methyl-phenyl]phenyl]indan-1-yl]-N-[[(2S)-5-oxopyrrolidin-2-yl]methyl]carbamate (70 mg, 83.1%). MS: m/z found 703.4 [M+H]⁺.

### tert-Butyl N-[5-[2-chloro-3-[2-methyl-3-[4-oxo-3-[[[(2S)-5-oxopyrrolidin-2-yl]methylamino]methyl]pyrido[1,2-a]pyrimidin-8-yl]phenyl]phenyl]indan-1-yl]-N-[[(2S)-5-oxopyrrolidin-2-yl]methyl]carbamate

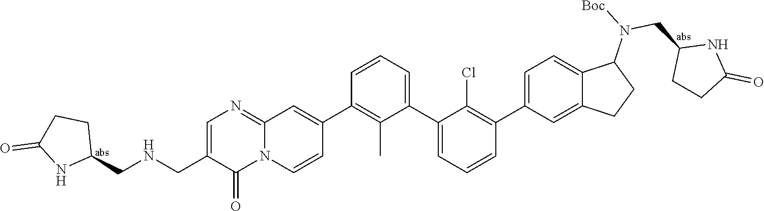 [SMILES: Cc1c(-c2ccn3c(=O)c(CNC[C@@H]4CCC(=O)N4)cnc3c2)cccc1-c1cccc(-c2ccc3c(c2)CCC3N(C[C@@H]2CCC(=O)N2)C(=O)OC(C)(C)C)c1Cl]

A mixture of tert-butyl N-[5-[2-chloro-3-[3-(3-formyl-4-oxo-pyrido[1,2-a]pyrimidin-8-yl)-2-methyl-phenyl]phenyl]indan-1-yl]-N-[[(2S)-5-oxopyrrolidin-2-yl]methyl]carbamate (50 mg, 0.07 mmol), (5S)-5-(aminomethyl)pyrrolidin-2-one (16 mg, 0.14 mmol) and acetic acid (4 uL, 4 mg, 0.07 mmol) in MeOH/THF (1:1) was stirred for 2 hour at rt. NaBH₃CN (9 mg, 0.14 mmol) was added, the mixture was stirred for additional 10 min, quenched with water and directly purified by HPLC. The collected fraction was neutralized with sat. NaHCO₃(aq.), acetonitrile was evaporated, and aqueous phase was extracted into DCM and EtOAc three times. The combined organic extracts were dried over anhydrous magnesium sulfate and concentrated in vacuo to give tert-butyl N-[5-[2-chloro-3-[2-methyl-3-[4-oxo-3-[[[(2S)-5-oxopyrrolidin-2-yl]methylamino]methyl]pyrido[1,2-a]pyrimidin-8-yl]phenyl]phenyl]indan-1-yl]-N-[[(2S)-5-oxopyrrolidin-2-yl]methyl]carbamate (40 mg, 70.2%). MS: m/z found 801.4 [M+H]⁺.

### 8-[3-[2-Chloro-3-[1-[[(2S)-5-oxopyrrolidin-2-yl]methylamino]indan-5-yl]phenyl]-2-methyl-phenyl]-3-[[[(2S)-5-oxopyrrolidin-2-yl]methylamino]methyl]pyrido[1,2-a]pyrimidin-4-one

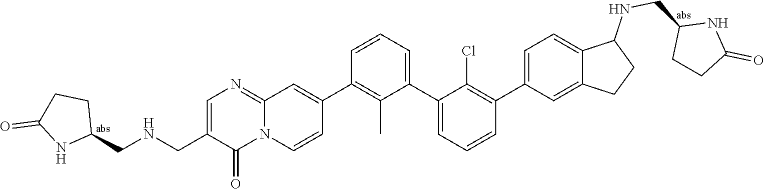 [SMILES: Cc1c(-c2ccn3c(=O)c(CNC[C@@H]4CCC(=O)N4)cnc3c2)cccc1-c1cccc(-c2ccc3c(c2)CCC3NC[C@@H]2CCC(=O)N2)c1Cl]

tert-Butyl N-[5-[2-chloro-3-[2-methyl-3-[4-oxo-3-[[[(2S)-5-oxopyrrolidin-2-yl]methylamino]methyl]pyrido[1,2-a]pyrimidin-8-yl]phenyl]phenyl]indan-1-yl]-N-[[(2S)-5-oxopyrrolidin-2-yl]methyl]carbamate (30 mg, 0.04 mmol) in DCM/TFA (1:1) was stirred for 30 min, concentrated, and purified by reversed phase chromatography to give 8-[3-[2-chloro-3-[1-[[(2S)-5-oxopyrrolidin-2-yl]methylamino]indan-5-yl]phenyl]-2-methyl-phenyl]-3-[[[(2S)-5-oxopyrrolidin-2-yl]methylamino]methyl]pyrido[1,2-a]pyrimidin-4-one (24 mg, 91.4%). The collected fraction was lyophilized and converted into HCl salt by the treatment with 3M HCl in MeOH. MS: m/z found 701.4 [M+H]⁺; ¹H NMR (400 MHz, Methanol-d₄): δ9.40 (d, J=7.2 Hz, 1H), 8.80 (s, 1H), 8.05 (d, J=1.6 Hz, 1H), 8.00 (dd, J=7.1, 1.7 Hz, 1H), 7.78 (dd, J=8.0, 2.8 Hz, 1H), 7.56-7.52 (m, 2H), 7.51 (d, J=7.6 Hz, 1H), 7.48 (s, 1H), 7.43 (ddd, J=6.3, 4.9, 2.3 Hz, 3H), 7.37 (dd, J=7.5, 1.7 Hz, 1H), 4.95 (dd, J=9.7, 5.4 Hz, 1H), 4.44 (d, J=2.2 Hz, 2H), 4.11 (dp, J=18.1, 6.2 Hz, 2H), 3.41-3.22 (m, 5H), 3.14-3.03 (m, 1H), 2.68 (dq, J=15.2, 7.7 Hz, 1H), 2.41 (tdd, J=18.5, 9.3, 6.0 Hz, 7H), 2.19 (s, 3H), 2.04-1.89 (m, 2H).

### Example 77: 8-(2′-Chloro-3′-(4-methoxy-5-(((((S)-5-oxopyrrolidin-2-yl)methyl)amino)methyl)pyridin-2-yl)-2-methyl-[1,1′-biphenyl]-3-yl)-3-(((((S)-5-oxopyrrolidin-2-yl)methyl)amino)methyl)-4H-pyrido[1,2-a]pyrimidin-4-one

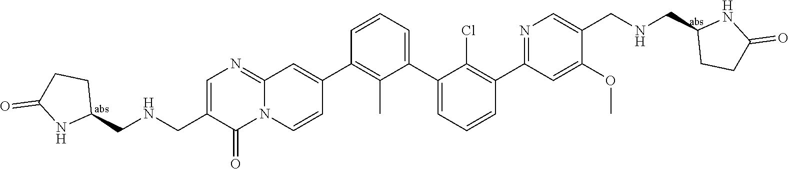 [SMILES: COc1cc(-c2cccc(-c3cccc(-c4ccn5c(=O)c(CNC[C@@H]6CCC(=O)N6)cnc5c4)c3C)c2Cl)ncc1CNC[C@@H]1CCC(=O)N1]

### 8-[3-[2-Chloro-3-(5-formyl-4-methoxy-2-pyridyl)phenyl]-2-methyl-phenyl]-4-oxo-pyrido[1,2-a]pyrimidine-3-carbaldehyde

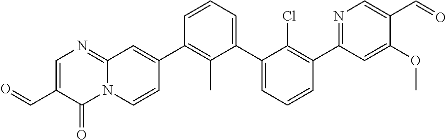 [SMILES: COc1cc(-c2cccc(-c3cccc(-c4ccn5c(=O)c(C=O)cnc5c4)c3C)c2Cl)ncc1C=O]

To a solution of 8-[3-[2-chloro-3-(4,4,5,5-tetramethyl-1,3,2-dioxaborolan-2-yl)phenyl]-2-methyl-phenyl]-4-oxo-pyrido[1,2-a]pyrimidine-3-carbaldehyde (60 mg, 0.12 mmol), 6-bromo-4-methoxy-pyridine-3-carbaldehyde (31 mg, 0.14 mmol) and potassium carbonate (50 mg, 0.36 mmol) in degassed dioxane/water (5:1) was added Pd(PPh₃)₄ (14 mg, 0.01 mmol) and the mixture was stirred at 90° C. for 3 hours under N₂(g). Water was added and the mixture was extracted into EtOAc three times. The combined organic layers were dried over anhydrous sodium sulfate, concentrated, and purified on silica gel column using EtOAc in hexanes (0 to 60% gradient) to give 8-[3-[2-chloro-3-(5-formyl-4-methoxy-2-pyridyl)phenyl]-2-methyl-phenyl]-4-oxo-pyrido[1,2-a]pyrimidine-3-carbaldehyde (29 mg, 47.5%). MS: m/z found 510.2 [M+H]⁺.

### 8-[3-[2-Chloro-3-[4-methoxy-5-[[[(2S)-5-oxopyrrolidin-2-yl]methylamino]methyl]-2-pyridyl]phenyl]-2-methyl-phenyl]-3-[[[(2S)-5-oxopyrrolidin-2-yl]methylamino]methyl]pyrido[1,2-a]pyrimidin-4-one

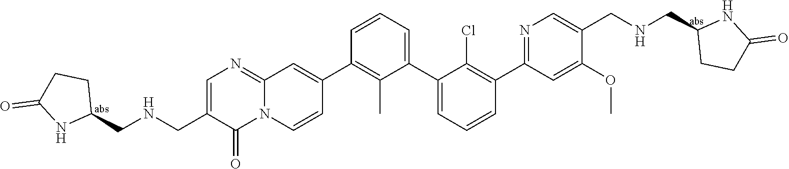 [SMILES: COc1cc(-c2cccc(-c3cccc(-c4ccn5c(=O)c(CNC[C@@H]6CCC(=O)N6)cnc5c4)c3C)c2Cl)ncc1CNC[C@@H]1CCC(=O)N1]

A mixture of 8-[3-[2-chloro-3-(5-formyl-4-methoxy-2-pyridyl)phenyl]-2-methyl-phenyl]-4-oxo-pyrido[1,2-a]pyrimidine-3-carbaldehyde (28 mg, 0.05 mmol), (5S)-5-(aminomethyl)pyrrolidin-2-one (25 mg, 0.22 mmol) and acetic acid (6 uL, 7 mg, 0.11 mmol) in MeOH/THF (1:1) was stirred for 1 hour at rt. NaBH₃CN (14 mg, 0.22 mmol) was added, the mixture was stirred for additional 9 h, quenched with water and directly purified by HPLC. The collected fraction was neutralized with sat. NaHCO₃(aq.), acetonitrile was evaporated, and aqueous phase was extracted into DCM and EtOAc three times. The combined organic extracts were dried over anhydrous magnesium sulfate and concentrated in vacuo to give 8-[3-[2-chloro-3-[4-methoxy-5-[[[(2S)-5-oxopyrrolidin-2-yl]methylamino]methyl]-2-pyridyl]phenyl]-2-methyl-phenyl]-3-[[[(2S)-5-oxopyrrolidin-2-yl]methylamino]methyl]pyrido[1,2-a]pyrimidin-4-one (24 mg, 61.9%). MS: m/z found 706.5 [M+H]⁺; ¹H NMR (400 MHz, Methanol-d₄): δ 9.13 (dd, J=7.3, 0.7 Hz, 1H), 8.41 (d, J=1.2 Hz, 2H), 7.65 (dd, J=1.8, 0.8 Hz, 1H), 7.57-7.52 (m, 2H), 7.48-7.41 (m, 4H), 7.32 (dd, J=5.8, 3.3 Hz, 1H), 7.27 (s, 1H), 3.99 (s, 3H), 3.90-3.82 (m, 6H), 2.79-2.64 (m, 4H), 2.37-2.24 (m, 6H), 2.14 (s, 3H), 1.87-1.77 (m, 2H).

### Example 78: 8-(2′-Chloro-2-methyl-3′-(5-(((((S)-5-oxopyrrolidin-2-yl)methyl)amino)methyl)-6-(trifluoromethyl)pyridin-2-yl)-[1,1′-biphenyl]-3-yl)-3-(((((S)-5-oxopyrrolidin-2-yl)methyl)amino)methyl)-4H-pyrido[1,2-a]pyrimidin-4-one

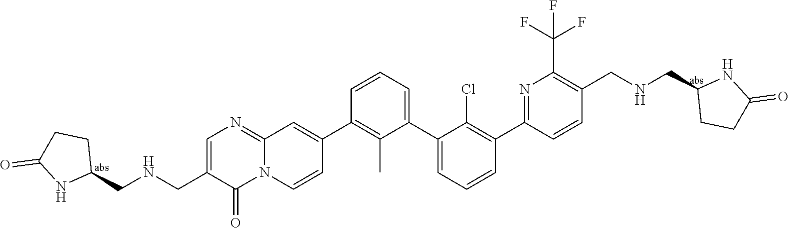 [SMILES: Cc1c(-c2ccn3c(=O)c(CNC[C@@H]4CCC(=O)N4)cnc3c2)cccc1-c1cccc(-c2ccc(CNC[C@@H]3CCC(=O)N3)c(C(F)(F)F)n2)c1Cl]

### 8-[3-[2-Chloro-3-[5-formyl-6-(trifluoromethyl)-2-pyridyl]phenyl]-2-methyl-phenyl]-4-oxo-pyrido[1,2-a]pyrimidine-3-carbaldehyde

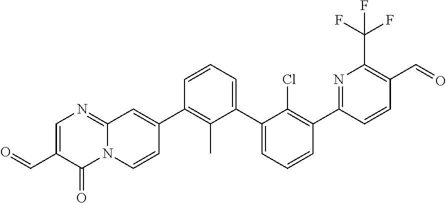 [SMILES: Cc1c(-c2ccn3c(=O)c(C=O)cnc3c2)cccc1-c1cccc(-c2ccc(C=O)c(C(F)(F)F)n2)c1Cl]

To a solution of 8-[3-[2-chloro-3-(4,4,5,5-tetramethyl-1,3,2-dioxaborolan-2-yl)phenyl]-2-methyl-phenyl]-4-oxo-pyrido[1,2-a]pyrimidine-3-carbaldehyde (60 mg, 0.12 mmol), 6-chloro-2-(trifluoromethyl)pyridine-3-carbaldehyde (30.13 mg, 0.14 mmol) and potassium carbonate (50 mg, 0.36 mmol) in degassed dioxane/water (5:1) was added Pd(PPh₃)₄ (14 mg, 0.01 mmol) and the mixture was stirred at 95° C. for overnight under N₂(g). Water was added and the mixture was extracted into EtOAc three times. The combined organic layers were dried over anhydrous sodium sulfate, concentrated, and purified on silica gel column using EtOAc in hexanes (0 to 60% gradient) to provide 8-[3-[2-chloro-3-[5-formyl-6-(trifluoromethyl)-2-pyridyl]phenyl]-2-methyl-phenyl]-4-oxo-pyrido[1,2-a]pyrimidine-3-carbaldehyde (35 mg, 53.3%). MS: m/z found 548.2 [M+H]⁺.

### 8-[3-[2-Chloro-3-[5-[[[(2S)-5-oxopyrrolidin-2-yl]methylamino]methyl]-6-(trifluoromethyl)-2-pyridyl]phenyl]-2-methyl-phenyl]-3-[[[(2S)-5-oxopyrrolidin-2-yl]methylamino]methyl]pyrido[1,2-a]pyrimidin-4-one

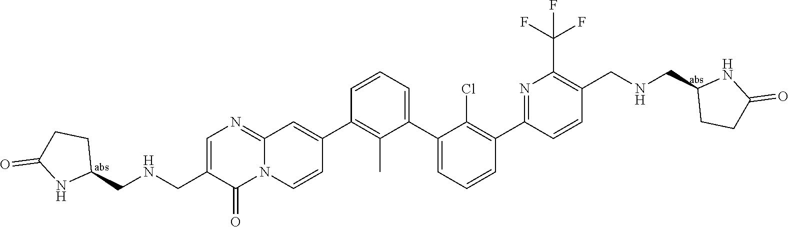 [SMILES: Cc1c(-c2ccn3c(=O)c(CNC[C@@H]4CCC(=O)N4)cnc3c2)cccc1-c1cccc(-c2ccc(CNC[C@@H]3CCC(=O)N3)c(C(F)(F)F)n2)c1Cl]

A mixture of 8-[3-[2-chloro-3-[5-formyl-6-(trifluoromethyl)-2-pyridyl]phenyl]-2-methyl-phenyl]-4-oxo-pyrido[1,2-a]pyrimidine-3-carbaldehyde (28 mg, 0.05 mmol), (5S)-5-(aminomethyl)pyrrolidin-2-one (23 mg, 0.20 mmol) and acetic acid (6 uL, 6 mg, 0.10 mmol) in MeOH/THF (1:1) was stirred for 1 hour at rt. NaBH₃CN (13 mg, 0.20 mmol) was added, the mixture was stirred for additional 5 h and quenched with water and directly purified by HPLC. The collected fraction was neutralized with sat. NaHCO₃(aq.), acetonitrile was evaporated, and aqueous phase was extracted into DCM and EtOAc three times. The combined organic extracts were dried over anhydrous magnesium sulfate and concentrated in vacuo to give 8-[3-[2-chloro-3-[5-[[[(2S)-5-oxopyrrolidin-2-yl]methylamino]methyl]-6-(trifluoromethyl)-2-pyridyl]phenyl]-2-methyl-phenyl]-3-[[[(2S)-5-oxopyrrolidin-2-yl]methylamino]methyl]pyrido[1,2-a]pyrimidin-4-one (33 mg, 86.8%). MS: m/z found 744.3 [M+H]⁺; ¹H NMR (400 MHz, Methanol-d₄): δ 9.13 (dt, J=7.3, 0.7 Hz, 1H), 8.40 (d, J=0.6 Hz, 1H), 8.30 (d, J=7.9 Hz, 1H), 7.94 (d, J=8.1 Hz, 1H), 7.65 (dd, J=1.9, 0.7 Hz, 1H), 7.62 (ddd, J=7.7, 1.8, 0.6 Hz, 1H), 7.55 (td, J=7.6, 0.6 Hz, 1H), 7.47-7.41 (m, 4H), 7.35-7.32 (m, 1H), 4.07 (dd, J=3.6, 1.6 Hz, 2H), 3.89-3.79 (m, 4H), 2.80-2.73 (m, 2H), 2.73-2.66 (m, 2H), 2.38-2.31 (m, 4H), 2.31-2.22 (m, 2H), 2.14 (s, 3H), 1.90-1.77 (m, 2H).

### Example 79: 8-(2′-Chloro-2-methyl-3′-(6-methyl-5-(((((S)-5-oxopyrrolidin-2-yl)methyl)amino)methyl)pyridin-2-yl)-[1,1′-biphenyl]-3-yl)-3-(((((S)-5-oxopyrrolidin-2-yl)methyl)amino)methyl)-4H-pyrido[1,2-a]pyrimidin-4-one

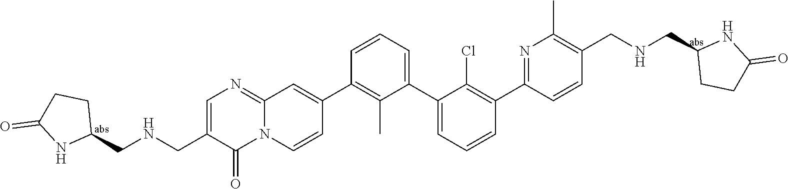 [SMILES: Cc1nc(-c2cccc(-c3cccc(-c4ccn5c(=O)c(CNC[C@@H]6CCC(=O)N6)cnc5c4)c3C)c2Cl)ccc1CNC[C@@H]1CCC(=O)N1]

### 8-[3-[2-Chloro-3-(5-formyl-6-methyl-2-pyridyl)phenyl]-2-methyl-phenyl]-4-oxo-pyrido[1,2-a]pyrimidine-3-carbaldehyde

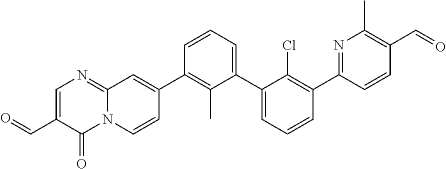 [SMILES: Cc1nc(-c2cccc(-c3cccc(-c4ccn5c(=O)c(C=O)cnc5c4)c3C)c2Cl)ccc1C=O]

To a solution of 8-[3-[2-chloro-3-(4,4,5,5-tetramethyl-1,3,2-dioxaborolan-2-yl)phenyl]-2-methyl-phenyl]-4-oxo-pyrido[1,2-a]pyrimidine-3-carbaldehyde (60 mg, 0.12 mmol), 6-bromo-2-methyl-pyridine-3-carbaldehyde (29 mg, 0.14 mmol) and potassium carbonate (50 mg, 0.36 mmol) in degassed dioxane/water (5:1) was added Pd(PPh₃)₄ (14 mg, 0.01 mmol) and the mixture was stirred at 95° C. for overnight under N₂(g). Water was added and the mixture was extracted into EtOAc three times. The combined organic layers were dried over anhydrous sodium sulfate, concentrated, and purified on silica gel column using EtOAc in hexanes (0 to 60% gradient). MS: m/z found 494.1 [M+H]⁺.

### 8-[3-[2-Chloro-3-[6-methyl-5-[[[(2S)-5-oxopyrrolidin-2-yl]methylamino]methyl]-2-pyridyl]phenyl]-2-methyl-phenyl]-3-[[[(2S)-5-oxopyrrolidin-2-yl]methylamino]methyl]pyrido[1,2-a]pyrimidin-4-one

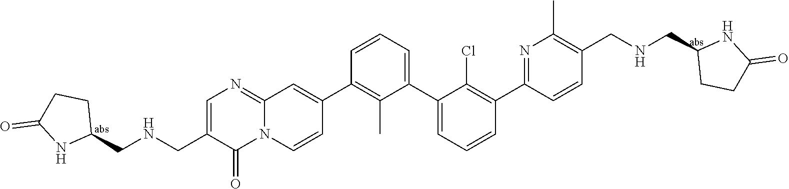 [SMILES: Cc1nc(-c2cccc(-c3cccc(-c4ccn5c(=O)c(CNC[C@@H]6CCC(=O)N6)cnc5c4)c3C)c2Cl)ccc1CNC[C@@H]1CCC(=O)N1]

A mixture of 8-[3-[2-chloro-3-(5-formyl-6-methyl-2-pyridyl)phenyl]-2-methyl-phenyl]-4-oxo-pyrido[1,2-a]pyrimidine-3-carbaldehyde (28 mg, 0.06 mmol), (5S)-5-(aminomethyl)pyrrolidin-2-one (26 mg, 0.23 mmol) and acetic acid (6 uL, 7 mg, 0.11 mmol) in MeOH/THF (1:1) was stirred for 1 hour at rt. NaBH₃CN (14.25 mg, 0.23 mmol) was added, the mixture was stirred for additional 5 h, quenched with water and directly purified by HPLC. The collected fraction was neutralized with sat. NaHCO₃(aq.), acetonitrile was evaporated, and aqueous phase was extracted into DCM and EtOAc three times. The combined organic extracts were dried over anhydrous magnesium sulfate and concentrated in vacuo to give 8-[3-[2-chloro-3-[6-methyl-5-[[[(2S)-5-oxopyrrolidin-2-yl]methylamino]methyl]-2-pyridyl]phenyl]-2-methyl-phenyl]-3-[[[(2S)-5-oxopyrrolidin-2-yl]methylamino]methyl]pyrido[1,2-a]pyrimidin-4-one (32 mg, 81.8%). MS: m/z found 690.3 [M+H]⁺; ¹H NMR (400 MHz, Methanol-d₄): δ 9.12 (dd, J=7.4, 0.7 Hz, 1H), 8.40 (d, J=0.7 Hz, 1H), 7.87 (d, J=7.9 Hz, 1H), 7.64 (dd, J=1.9, 0.8 Hz, 1H), 7.53-7.49 (m, 2H), 7.46-7.42 (m, 4H), 7.42-7.39 (m, 1H), 7.31 (dd, J=6.3, 2.8 Hz, 1H), 3.89 (d, J=6.4 Hz, 2H), 3.88-3.81 (m, 4H), 2.83-2.75 (m, 2H), 2.75-2.66 (m, 2H), 2.62 (s, 3H), 2.38-2.31 (m, 4H), 2.31-2.23 (m, 2H), 2.13 (s, 3H), 1.91-1.76 (m, 2H).

### Example 80: (S)-8-(2,2′-Dichloro-3′-(6-methoxy-5-((((5-oxopyrrolidin-2-yl)methyl)amino)methyl)pyridin-2-yl)-[1,1′-biphenyl]-3-yl)-3-(((2-hydroxyethyl)amino)methyl)-4H-pyrido[1,2-a]pyrimidin-4-one

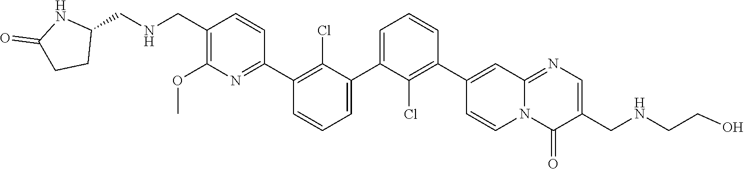 [SMILES: COc1nc(-c2cccc(-c3cccc(-c4ccn5c(=O)c(CNCCO)cnc5c4)c3Cl)c2Cl)ccc1CNC[C@@H]1CCC(=O)N1]

### (S)-5-((((6-(2,2′-Dichloro-3′-(4,4,5,5-tetramethyl-1,3,2-dioxaborolan-2-yl)-[1,1′-biphenyl]-3-yl)-2-methoxypyridin-3-yl)methyl)amino)methyl)pyrrolidin-2-one

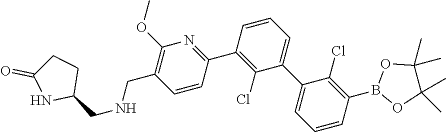 [SMILES: COc1nc(-c2cccc(-c3cccc(B4OC(C)(C)C(C)(C)O4)c3Cl)c2Cl)ccc1CNC[C@@H]1CCC(=O)N1]

6-[2-Chloro-3-[2-chloro-3-(4,4,5,5-tetramethyl-1,3,2-dioxaborolan-2-yl)phenyl]phenyl]-2-methoxy-pyridine-3-carbaldehyde (250 mg, 0.52 mmol), (5S)-5-(aminomethyl)-pyrrolidin-2-one (77 mg, 0.67 mmol) and acetic acid (47 mg, 0.77 mmol) were dissolved in 8 mL of 1:1 MeOH/THF solvent mix, and the reaction was stirred at 25° C. for 2 hours. NaBH₃CN (65 mg, 1.03 mmol), was then added portion-wise, and the resulting mixture was further stirred at 25° C. for 1 hour. The reaction was concentrated and the residue was dissolved in 10 ml water and the aqueous was extracted with EtOAc (3×10 mL). The combined organics were washed with 10 ml brine, dried over sodium sulfate, then filtered and concentrated to afford (5S)-5-[[[6-[2-chloro-3-[2-chloro-3-(4,4,5,5-tetramethyl-1,3,2-dioxaborolan-2-yl)phenyl]phenyl]-2-methoxy-3-pyridyl]methylamino]methyl]pyrrolidin-2-one (303 mg) as a white foam. Material was used as—is for the next step. MS: m/z found 582.4, 584 [M+H]⁺.

### tert-Butyl (S)-((6-(2,2′-dichloro-3′-(4,4,5,5-tetramethyl-1,3,2-dioxaborolan-2-yl)-[1,1′-biphenyl]-3-yl)-2-methoxypyridin-3-yl)-methyl)((5-oxopyrrolidin-2-yl)methyl)carbamate

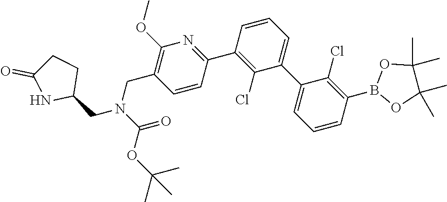 [SMILES: COc1nc(-c2cccc(-c3cccc(B4OC(C)(C)C(C)(C)O4)c3Cl)c2Cl)ccc1CN(C[C@@H]1CCC(=O)N1)C(=O)OC(C)(C)C]

(5S)-5-[[[6-[2-Chloro-3-[2-chloro-3-(4,4,5,5-tetramethyl-1,3,2-dioxaborolan-2-yl)phenyl]phenyl]-2-methoxy-3-pyridyl]methylamino]methyl]pyrrolidin-2-one (303 mg, 0.52 mmol) and DIEA (362 μL, 269 mg, 2.08 mmol) were dissolved in 10 ml anhydrous THE and di-tert-butyl dicarbonate, (227 mg, 1.04 mmol) was added portion-wise. The resulting mixture was stirred at rt for 12 hours. The reaction was diluted with EtOAc (20 ml) and the resulting solution was washed with aqueous saturated sodium bicarbonate solution (10 ml×2) and brine (15 ml×2). The organic layer was concentrated in vacuo to give a crude sample, which was purified by silica gel chromatography, eluting with 10-100% EtOAc gradient in hexane to afford tert-butyl N-[[6-[2-chloro-3-[2-chloro-3-(4,4,5,5-tetramethyl-1,3,2-dioxaborolan-2-yl)phenyl]phenyl]-2-methoxy-3-pyridyl]methyl]-N-[[(2S)-5-oxopyrrolidin-2-yl]methyl]carbamate (205 mg, 57.6% yield) as a white foam. MS: m/z found 682.4, 684.4 [M+H]⁺.

### tert-Butyl (S)-((6-(2,2′-dichloro-3′-(3-formyl-4-oxo-4H-pyrido[1,2-a]pyrimidin-8-yl)-[1,1′-biphenyl]-3-yl)-2-methoxypyridin-3-yl)methyl)((5-oxopyrrolidin-2-yl)methyl)carbamate

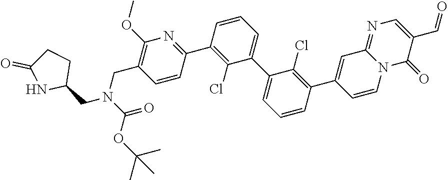 [SMILES: COc1nc(-c2cccc(-c3cccc(-c4ccn5c(=O)c(C=O)cnc5c4)c3Cl)c2Cl)ccc1CN(C[C@@H]1CCC(=O)N1)C(=O)OC(C)(C)C]

Pd(PPh₃)₄ (80 mg, 0.07 mmol), potassium carbonate (143 mg, 1.03 mmol), tert-butyl N-[[6-[2-chloro-3-[2-chloro-3-(4,4,5,5-tetramethyl-1,3,2-dioxaborolan-2-yl)phenyl]phenyl]-2-methoxy-3-pyridyl]methyl]-N-[[(2S)-5-oxopyrrolidin-2-yl]methyl]carbamate (294 mg, 0.34 mmol) and 8-bromo-4-oxo-pyrido[1,2-a]pyrimidine-3-carbaldehyde (105 mg, 0.41 mmol) were suspended in 10 ml of 4:1 dioxane/water solvent mix, and the reaction was heated at 105° C. for 20 minutes. The solution was subsequently cooled to rt, diluted with 25 mL water, and extracted with EtOAc (3×20 mL). The combined organics were concentrated and crude sample was purified by silica chromatography, eluting with 0-3% MeOH gradient in DCM to afford tert-butyl N-[[6-[2-chloro-3-[2-chloro-3-(3-formyl-4-oxo-pyrido[1,2-a]pyrimidin-8-yl)phenyl]phenyl]-2-methoxy-3-pyridyl]-methyl]-N-[[(2S)-5-oxopyrrolidin-2-yl]methyl]carbamate (228 mg, 90.8% yield) as a yellow foam. MS: m/z found 728.3, 730.3 [M+H]⁺.

### tert-Butyl (S)-((6-(2,2′-dichloro-3′-(3-(((2-hydroxyethyl)amino)methyl)-4-oxo-4H-pyrido[1,2-a]pyrimidin-8-yl)-[1,1′-biphenyl]-3-yl)-2-methoxypyridin-3-yl)methyl)((5-oxopyrrolidin-2-yl)methyl)carbamate

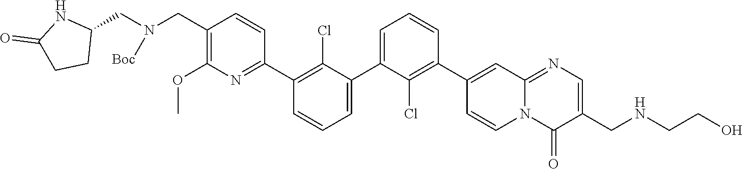 [SMILES: COc1nc(-c2cccc(-c3cccc(-c4ccn5c(=O)c(CNCCO)cnc5c4)c3Cl)c2Cl)ccc1CN(C[C@@H]1CCC(=O)N1)C(=O)OC(C)(C)C]

tert-Butyl N-[[6-[2-chloro-3-[2-chloro-3-(3-formyl-4-oxo-pyrido[1,2-a]pyrimidin-8-yl)phenyl]phenyl]-2-methoxy-3-pyridyl]methyl]-N-[[(2S)-5-oxopyrrolidin-2-yl]methyl]carbamate (19 mg, 0.03 mmol), 2-aminoethanol (5 mg, 0.08 mmol) and acetic acid (3 mg, 0.05 mmol) were dissolved in 1 ml of 1:1 MeOH/THF solvent mix, and the reaction was stirred at 25° C. for 3 hours. NaBH₃CN (3 mg, 0.05 mmol) was added portion-wise, and the resulting mixture was stirred at 25° C. for 1 hour. The reaction was concentrated and the residue was dissolved in water (3 mL), then extracted with DCM (3×2 mL). The combined organics were further washed with brine, dried over sodium sulfate, then filtered and concentrated to afford tert-butyl N-[[6-[2-chloro-3-[2-chloro-3-[3-[(2-hydroxyethylamino)methyl]-4-oxo-pyrido[1,2-a]pyrimidin-8-yl]phenyl]phenyl]-2-methoxy-3-pyridyl]methyl]-N-[[(2S)-5-oxopyrrolidin-2-yl]methyl]carbamate (23 mg) as an orange oil. Material was used as—is for the next step. MS: m/z found 773.4, 775.4 [M+H]⁺.

### (S)-8-(2,2′-Dichloro-3′-(6-methoxy-5-((((5-oxopyrrolidin-2-yl)methyl)amino)methyl)pyridin-2-yl)-[1,1′-biphenyl]-3-yl)-3-(((2-hydroxyethyl)amino)methyl)-4H-pyrido[1,2-a]pyrimidin-4-one

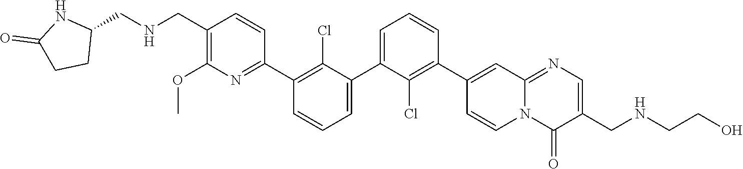 [SMILES: COc1nc(-c2cccc(-c3cccc(-c4ccn5c(=O)c(CNCCO)cnc5c4)c3Cl)c2Cl)ccc1CNC[C@@H]1CCC(=O)N1]

tert-Butyl N-[[6-[2-chloro-3-[2-chloro-3-[3-[(2-hydroxyethylamino)methyl]-4-oxo-pyrido[1,2-a]pyrimidin-8-yl]phenyl]phenyl]-2-methoxy-3-pyridyl]methyl]-N-[[(2S)-5-oxopyrrolidin-2-yl]methyl]carbamate (23 mg, 0.03 mmol) was dissolved in 0.5 mL of DCM and 4M HCl solution in dioxane (22 μL, 0.09 mmol) was added. The reaction was stirred at rt for 20 minutes and the mixture was subsequently concentrated. The crude residue was purified by reverse phase HPLC to afford 8-[2-chloro-3-[2-chloro-3-[6-methoxy-5-[[[(2S)-5-oxopyrrolidin-2-yl]methylamino]methyl]-2-pyridyl]phenyl]phenyl]-3-[(2-hydroxyethylamino)methyl]pyrido[1,2-a]pyrimidin-4-one (7 mg, 35.0% yield) as a light yellow foam. MS: m/z found 673.4, 675.4 [M+H]⁺. ¹H NMR (400 MHz, Chloroform-d) δ 9.08 (d, J=7.5 Hz, 1H), 8.36 (s, 1H), 7.76 (d, J=1.9 Hz, 1H), 7.71-7.66 (m, 1H), 7.59 (d, J=7.5 Hz, 1H), 7.51-7.42 (m, 4H), 7.38 (dd, J=7.5, 1.9 Hz, 1H), 7.33 (dd, J=7.6, 1.7 Hz, 1H), 7.28 (s, 1H), 6.01 (d, J=5.5 Hz, 1H), 4.03 (d, J=1.3 Hz, 3H), 3.92 (s, 2H), 3.81 (s, 3H), 3.74-3.70 (m, 2H), 2.89-2.84 (m, 2H), 2.80 (dd, J=12.0, 4.2 Hz, 1H), 2.57 (dd, J=12.0, 8.6 Hz, 1H), 2.36 (t, J=8.1 Hz, 2H), 2.29-2.19 (m, 1H), 1.77 (dd, J=13.4, 6.2 Hz, 1H).

### Example 81: (S)—N-(2-(((8-(2,2′-Dichloro-3′-(6-methoxy-5-((((5-oxopyrrolidin-2-yl)methyl)amino)methyl)pyridin-2-yl)-[1,1′-biphenyl]-3-yl)-4-oxo-4H-pyrido[1,2-a]pyrimidin-3-yl)methyl)amino)ethyl)acetamide

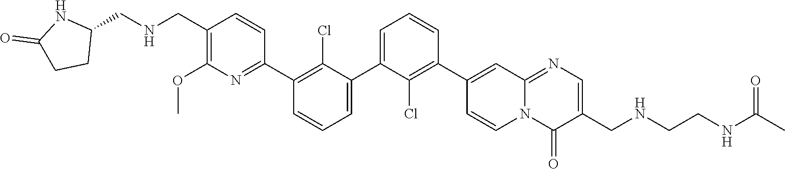 [SMILES: COc1nc(-c2cccc(-c3cccc(-c4ccn5c(=O)c(CNCCNC(C)=O)cnc5c4)c3Cl)c2Cl)ccc1CNC[C@@H]1CCC(=O)N1]

tert-Butyl N-[[6-[2-chloro-3-[2-chloro-3-(3-formyl-4-oxo-pyrido[1,2-a]pyrimidin-8-yl)phenyl]phenyl]-2-methoxy-3-pyridyl]methyl]-N-[[(2S)-5-oxopyrrolidin-2-yl]methyl]carbamate (19 mg, 0.03 mmol), N-(2-aminoethyl)acetamide (8 mg, 0.08 mmol) and acetic acid (3 mg, 0.05 mmol) were dissolved in 1 ml of 1:1 MeOH/THF solvent mix, and the reaction was stirred at 25° C. for 3 hours. NaBH₃CN (3 mg, 0.05 mmol) was added portion-wise, and the resulting mixture was stirred at 25° C. for 1 hour. The reaction was concentrated and the residue was dissolved in water (3 ml), then extracted with DCM (3×2 mL). The combined organics were further washed with brine, dried over sodium sulfate, then filtered and concentrated to afford tert-butyl N-[[6-[3-[3-[3-[(2-acetamidoethylamino)methyl]-4-oxo-pyrido[1,2-a]pyrimidin-8-yl]-2-chloro-phenyl]-2-chloro-phenyl]-2-methoxy-3-pyridyl]methyl]-N-[[(2S)-5-oxopyrrolidin-2-yl]methyl]carbamate (17.4 mg) as an orange oil. MS: m/z found 814.4, 816.4 [M+H]⁺. The material was used for next reaction directly.

tert-butyl N-[[6-[3-[3-[3-[(2-acetamidoethylamino)methyl]-4-oxo-pyrido[1,2-a]pyrimidin-8-yl]-2-chloro-phenyl]-2-chloro-phenyl]-2-methoxy-3-pyridyl]methyl]-N-[[(2S)-5-oxopyrrolidin-2-yl]methyl]carbamate (17.4 mg, 0.02 mmol) was dissolved in 0.5 ml DCM and 4M HCl solution in dioxane (16 μL, 0.06 mmol) was added. The reaction was stirred at rt for 20 minutes and the mixture was subsequently concentrated. Crude sample was purified by reverse phase HPLC to afford N-[2-[[8-[2-chloro-3-[2-chloro-3-[6-methoxy-5-[[[(2S)-5-oxopyrrolidin-2-yl]methylamino]methyl]-2-pyridyl]phenyl]phenyl]-4-oxo-pyrido[1,2-a]pyrimidin-3-yl]methylamino]ethyl]acetamide (6 mg, 39.3% yield) as a clear film. MS: m/z found 714.3, 716 [M+H]⁺. ¹H NMR (400 MHz, Chloroform-d) δ 9.08 (d, J=7.4 Hz, 1H), 8.34 (s, 1H), 7.76 (d, J=1.3 Hz, 1H), 7.69 (d, J=7.8 Hz, 1H), 7.59 (d, J=7.4 Hz, 1H), 7.52-7.43 (m, 4H), 7.39 (dd, J=7.5, 1.9 Hz, 1H), 7.33 (dd, J=7.6, 1.6 Hz, 1H), 6.37 (s, 1H), 5.99 (s, 1H), 4.03 (d, J=1.0 Hz, 3H), 3.87 (s, 2H), 3.81 (s, 3H), 3.40 (q, J=5.6 Hz, 2H), 2.84-2.77 (m, 3H), 2.57 (dd, J=12.0, 8.7 Hz, 1H), 2.37 (dd, J=9.1, 7.0 Hz, 2H), 2.24 (dq, J=14.9, 7.3 Hz, 1H), 2.01 (d, J=1.0 Hz, 3H).

### Example 82: (S)-5-((((6-(2,2′-Dichloro-3′-(2-(((((S)-5-oxopyrrolidin-2-yl)methyl)amino)methyl)pyrazolo[1,5-a]pyrimidin-5-yl)-[1,1′-biphenyl]-3-yl)-2-methoxypyridin-3-yl)methyl)amino)methyl)pyrrolidin-2-one

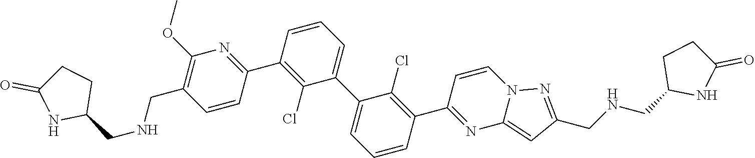 [SMILES: COc1nc(-c2cccc(-c3cccc(-c4ccn5nc(CNC[C@@H]6CCC(=O)N6)cc5n4)c3Cl)c2Cl)ccc1CNC[C@@H]1CCC(=O)N1]

### (5-Chloropyrazolo[1,5-a]-pyrimidin-2-yl)-methanol

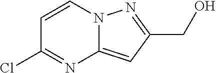 [SMILES: OCc1cc2nc(Cl)ccn2n1]

Ethyl 5-chloropyrazolo[1,5-a]-pyrimidine-2-carboxylate (0.3 g, 1.33 mmol) was dissolved in 5 ml anhydrous THE and the solution was cooled to −10° C. A solution of 1M di-isobutylaluminum hydride in THE (3.06 ml, 0.44 g, 3.06 mmol) was added dropwise over 15 minutes under a nitrogen stream, and the resulting mixture was stirred at −10° C. for 1 hour. The reaction was then warmed to rt and stirred for an additional 12 hours. The reaction was added slowly to an ice water solution with vigorous stirring and the precipitate was filtered. The aqueous filtrate was concentrated, and the crude oil was purified by reverse phase HPLC to afford (5-chloropyrazolo[1,5-a]-pyrimidin-2-yl)-methanol (54 mg, 22% yield) as a yellow foam. MS: m/z found 184, 186 [M+H]⁺. ¹H NMR (400 MHz, Chloroform-d) δ 8.52 (d, J=7.2 Hz, 1H), 6.90-6.72 (m, 1H), 6.60 (s, 1H), 4.91 (s, 2H).

### 5-Chloropyrazolo[1,5-a]-pyrimidine-2-carbaldehyde

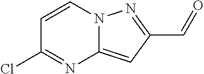 [SMILES: O=Cc1cc2nc(Cl)ccn2n1]

Dess-Martin periodinane (0.37 g, 0.88 mmol) was added portion-wise over 2 minutes to a solution of (5-chloropyrazolo[1,5-a]-pyrimidin-2-yl)-methanol (54 mg, 0.29 mmol) in 5 mL DCM and 0.3 mL DMF solvent mix. The reaction was stirred at rt for 12 hours, then the solution was concentrated to remove all the DCM. The remaining solution was diluted with water (10 ml) and extracted with EtOAc (3×3 mL). The combined organics were concentrated, and the crude residue was purified by silica gel chromatography, eluting with 0-25% EtOAc gradient in hexane to afford 5-chloropyrazolo[1,5-a]-pyrimidine-2-carbaldehyde (32.5 mg, 60.9% yield) as a white solid. MS: m/z found 182, 184 [M+H]⁺. ¹H NMR (400 MHz, Chloroform-d) δ 10.17 (d, J=1.9 Hz, 1H), 8.66-8.57 (m, 1H), 7.11 (t, J=1.5 Hz, 1H), 6.99 (dd, J=7.3, 1.7 Hz, 1H).

### 5-(2,2′-Dichloro-3′-(5-formyl-6-methoxypyridin-2-yl)-[1,1′-biphenyl]-3-yl)-pyrazolo[1,5-a]-pyrimidine-2-carbaldehyde

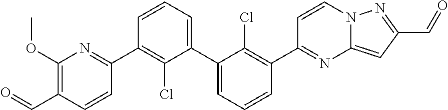 [SMILES: COc1nc(-c2cccc(-c3cccc(-c4ccn5nc(C=O)cc5n4)c3Cl)c2Cl)ccc1C=O]

1,1′-Bis(diphenylphosphino)-ferrocene-palladium(ii) dichloride dichloromethane complex (30 mg, 0.04 mmol), potassium carbonate (102 mg, 0.74 mmol), 2-[2-chloro-3-[2-chloro-3-(4,4,5,5-tetramethyl-1,3,2-dioxaborolan-2-yl)phenyl]phenyl]-4,4,5,5-tetramethyl-1,3,2-dioxaborolane (88 mg, 0.19 mmol), 5-chloropyrazolo[1,5-a]pyrimidine-2-carbaldehyde (34 mg, 0.19 mmol) and 6-chloro-2-methoxy-pyridine-3-carbaldehyde (32 mg, 0.19 mmol) were suspended in 3 mL of 9:1 dioxane/water solvent mix. The solution was purged with nitrogen gas for 5 minutes, then heated at 95° C. for 1 hour. The reaction was cooled to rt, filtered through CELITE®, and the filtrate was concentrated. Crude sample was purified by silica gel chromatography, eluting with 0-50% EtOAc gradient in hexane to afford 5-[2-chloro-3-[2-chloro-3-(5-formyl-6-methoxy-2-pyridyl)phenyl]phenyl]pyrazolo[1,5-a]pyrimidine-2-carbaldehyde (12 mg, 12.9% yield) as a white solid. MS: m/z found 503.1, 505.1 [M+H]⁺.

### (S)-5-((((6-(2,2′-Dichloro-3′-(2-(((((S)-5-oxopyrrolidin-2-yl)methyl)amino)methyl)pyrazolo[1,5-a]pyrimidin-5-yl)-[1,1′-biphenyl]-3-yl)-2-methoxypyridin-3-yl)methyl)amino)methyl)pyrrolidin-2-one

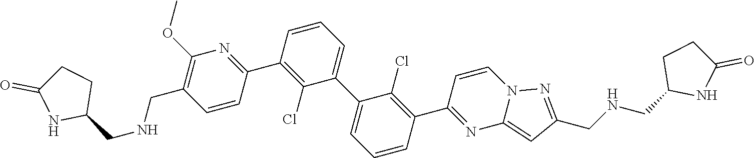 [SMILES: COc1nc(-c2cccc(-c3cccc(-c4ccn5nc(CNC[C@@H]6CCC(=O)N6)cc5n4)c3Cl)c2Cl)ccc1CNC[C@@H]1CCC(=O)N1]

5-[2-Chloro-3-[2-chloro-3-(5-formyl-6-methoxy-2-pyridyl)phenyl]phenyl]pyrazolo[1,5-a]pyrimidine-2-carbaldehyde (12 mg, 0.02 mmol), (5S)-5-(aminomethyl)pyrrolidin-2-one (8 mg, 0.07 mmol) and acetic acid (4 mg, 0.06 mmol) were dissolved in 1 mL of 1:1 MeOH/THF solvent mix, and the reaction was stirred at 25° C. for 3 hours. NaBH₃CN (4.5 mg, 0.07 mmol) was then added portion-wise, and the resulting mixture was stirred at 25° C. for 1 hour. The reaction was concentrated and the crude sample was purified by reverse phase HPLC to afford (5S)-5-[[[6-[2-chloro-3-[2-chloro-3-[2-[[[(2S)-5-oxopyrrolidin-2-yl]methylamino]methyl]pyrazolo[1,5-a]pyrimidin-5-yl]phenyl]phenyl]-2-methoxy-3-pyridyl]methylamino]methyl]pyrrolidin-2-one (14 mg, 83.3% yield) as a white foam. MS: m/z found 699.2, 701.2 [M+H]⁺. ¹H NMR (400 MHz, Chloroform-d) δ 8.64 (d, J=7.3 Hz, 1H), 7.69 (ddd, J=7.7, 5.8, 1.8 Hz, 2H), 7.58 (d, J=7.5 Hz, 1H), 7.53-7.41 (m, 3H), 7.32 (dd, J=7.6, 1.7 Hz, 1H), 7.28 (s, 1H), 7.21 (d, J=7.3 Hz, 1H), 6.65 (s, 1H), 6.16 (s, 1H), 6.09 (s, 1H), 4.07 (s, 2H), 4.02 (s, 3H), 3.81 (s, 4H), 2.88 (dd, J=12.0, 4.1 Hz, 1H), 2.79 (dd, J=12.0, 4.2 Hz, 1H), 2.65 (dd, J=12.0, 8.5 Hz, 1H), 2.56 (dd, J=12.0, 8.6 Hz, 1H), 2.36 (ddd, J=9.8, 7.0, 4.7 Hz, 4H), 2.30-2.18 (m, 2H), 1.82-1.73 (m, 2H).

### Example 83: (S)-1-((8-(2,2′-Dichloro-3′-(5-((3-(hydroxymethyl)azetidin-1-yl)methyl)-6-methoxypyridin-2-yl)-[1,1′-biphenyl]-3-yl)-4-oxo-4H-pyrido[1,2-a]pyrimidin-3-yl)methyl)pyrrolidine-3-carboxylic acid

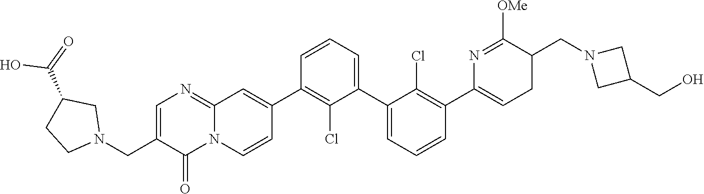 [SMILES: COC1=NC(c2cccc(-c3cccc(-c4ccn5c(=O)c(CN6CC[C@H](C(=O)O)C6)cnc5c4)c3Cl)c2Cl)=CCC1CN1CC(CO)C1]

### 6-(3-Bromo-2-chlorophenyl)-2-methoxynicotinaldehyde

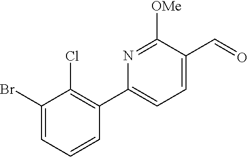 [SMILES: COc1nc(-c2cccc(Br)c2Cl)ccc1C=O]

To a mixture of 6-bromo-2-methoxynicotinaldehyde (2 g, 11.7 mmol) and 2-(3-bromo-2-chlorophenyl)-4,4,5,5-tetramethyl-1,3,2-dioxaborolane (3.51 g, 11.1 mmol) in dioxane/H₂O mixture (10:1, 44 mL) was added tetrakis(triphenylphosphine)palladium (0.67 g, 0.58 mmol) and potassium carbonate (4.83 g, 35 mmol) in one portion under N₂. The mixture was stirred at 95° C. for 3 h and then was concentrated. To the residue was added water (50 mL) and saturated aqueous brine solution (50 mL). The mixture was extracted with ethyl acetate/THF mixture (1:1, 200 mL). The organic phase was dried with anhydrous sodium sulfate, filtered and concentrated. The residue was purified by normal phase SiO₂ chromatography (0-30% CH₂Cl₂ in ethyl acetate/petroleum ether) to afford 6-(3-bromo-2-chlorophenyl)-2-methoxynicotinaldehyde as a white solid (3 g, 78% yield). ¹H NMR (400 MHz, DMSO-d₆): δ 10.30 (s, 1H), 8.22 (d, J=7.6 Hz, 1H), 7.92 (d, J=6.4 Hz, 1H), 7.65 (d, J=6 Hz, 1H), 7.46-7.43 (m, 2H), 4.04 (s, 3H).

### (1-((6-(3-Bromo-2-chlorophenyl)-2-methoxypyridin-3-yl)methyl)azetidin-3-yl)methanol

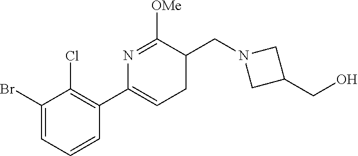 [SMILES: COC1=NC(c2cccc(Br)c2Cl)=CCC1CN1CC(CO)C1]

To a mixture of 6-(3-bromo-2-chlorophenyl)-2-methoxynicotinaldehyde (3 g, 9.19 mmol) and azetidin-3-ylmethanol HCl salt (1.7 g, 13.8 mmol) in CH₂Cl₂ (120 mL) was added sodium acetate (1.51 g, 18.4 mmol) and sodium triacetoxyborohydride (2.92 g, 13.8 mmol) in one portion under N₂. The mixture was stirred at r.t. for 2 h and concentrated. The residue was purified by reverse phase HPLC to afford (1-((6-(3-bromo-2-chlorophenyl)-2-methoxypyridin-3-yl)methyl)azetidin-3-yl)methanol as a light yellow gum (1.5 g, 39% yield). ¹H NMR (400 MHz, DMSO-d₆): δ 7.84 (dd, J=8 Hz, J=1.6 Hz, 1H), 7.69 (d, J=7.6 Hz, 1H), 7.57 (dd, J=8 Hz, J=1.6 Hz, 1H), 7.38 (t, J=8 Hz, 1H), 7.20 (d, J=7.2 Hz, 1H), 4.59 (t, J=5.2 Hz, 1H), 3.88 (s, 3H), 3.54-3.52 (m, 4H), 3.30-3.28 (m, 2H), 3.18 (d, J=4.8 Hz, 1H), 2.98 (t, J=6.4 Hz, 2H).

### 8-(2,2′-Dichloro-3′-(5-((3-(hydroxymethyl)azetidin-1-yl)methyl)-6-methoxypyridin-2-yl)-[1,1′-biphenyl]-3-yl)-4-oxo-4H-pyrido[1,2-a]pyrimidine-3-carbaldehyde

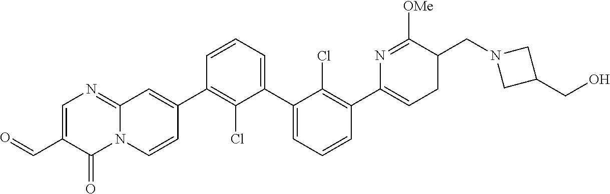 [SMILES: COC1=NC(c2cccc(-c3cccc(-c4ccn5c(=O)c(C=O)cnc5c4)c3Cl)c2Cl)=CCC1CN1CC(CO)C1]

To a mixture of(1-((6-(3-bromo-2-chlorophenyl)-2-methoxypyridin-3-yl)methyl)azetidin-3-yl)methanol (0.3 g, 0.75 mmol) and 8-(2-chloro-3-(4,4,5,5-tetramethyl-1,3,2-dioxaborolan-2-yl)phenyl)-4-oxo-4H-pyrido[1,2-a]pyrimidine-3-carbaldehyde (0.56 g, 1.36 mmol) in dioxane/H₂O mixture (10:1, 11 mL) was added potassium phosphate (0.48 g, 2.26 mmol) and ditert-butyl(cyclopentyl)phosphane;dichloropalladium;iron (0.05 g, 0.07 mmol) in one portion under N₂. The mixture was stirred at 110° C. for 0.5 h and concentrated. To the residue was added water (50 mL) and saturated aqueous brine solution (50 mL). The mixture was extracted with ethyl acetate/THF mixture (1:1, 100 mL). The organic phase was dried with anhydrous sodium sulfate, filtered and concentrated. The residue was purified by normal phase SiO₂ chromatography (60-100% ethyl acetate/petroleum ether to ethyl acetate/THF/MeOH=2/1/1) to afford 8-(2,2′-dichloro-3′-(5-((3-(hydroxymethyl)azetidin-1-yl)methyl)-6-methoxypyridin-2-yl)-[1,1′-biphenyl]-3-yl)-4-oxo-4H-pyrido[1,2-a]pyrimidine-3-carbaldehyde as a yellow solid (0.4 g, 81% yield, m/z: 601 [M+H]⁺). ¹H NMR (400 MHz, DMSO-d₆): δ 10.16 (s, 1H), 9.25 (d, J=6.8 Hz, 1H), 8.76 (s, 1H), 8.00 (d, J=1.6 Hz, 1H), 7.77-7.52 (m, 7H), 7.47-7.45 (m, 1H), 7.27 (d, J=7.6 Hz, 1H), 4.06 (t, J=5.2 Hz, 1H), 3.90 (s, 3H), 3.49 (m, 3H), 3.14-3.12 (m, 1H), 2.63 (m, 1H), 1.32 (s, 2H), 1.20-1.12 (m, 1H).

### (S)-1-((8-(2,2′-Dichloro-3′-(5-((3-(hydroxymethyl)azetidin-1-yl)methyl)-6-methoxypyridin-2-yl)-[1,1′-biphenyl]-3-yl)-4-oxo-4H-pyrido[1,2-a]pyrimidin-3-yl)methyl)pyrrolidine-3-carboxylic acid

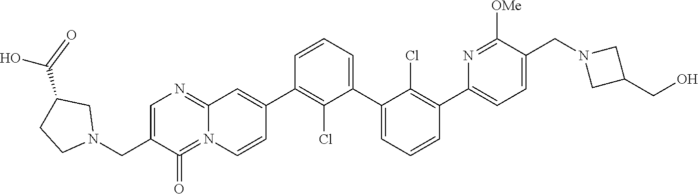

To a mixture of 8-(2,2′-dichloro-3′-(5-((3-(hydroxymethyl)azetidin-1-yl)methyl)-6-methoxypyridin-2-yl)-[1,1′-biphenyl]-3-yl)-4-oxo-4H-pyrido[1,2-a]pyrimidine-3-carbaldehyde (150 mg, 0.25 mmol) and (S)-pyrrolidine-3-carboxylic acid (0.04 g, 0.37 mmol) in CH₂Cl₂ (5 mL) was added sodium acetate (0.04 g, 0.5 mmol), 4 Å molecular sieve (0.1 g) and sodium triacetoxyborohydride (0.1 g, 0.5 mmol) in one portion under N₂. The mixture was stirred at r.t. for 5 hand then was filtered. The filter cake was washed with MeOH (20 mL). The filtrate was concentrated. The residue was purified by reverse phase HPLC to afford (S)-1-((8-(2,2′-dichloro-3′-(5-((3-(hydroxymethyl)azetidin-1-yl)methyl)-6-methoxypyridin-2-yl)-[1,1′-biphenyl]-3-yl)-4-oxo-4H-pyrido[1,2-a]pyrimidin-3-yl)methyl)pyrrolidine-3-carboxylic acid as a white solid (47 mg, 26% yield, m/z: 700 [M+H]⁺). ¹H NMR (400 MHz, Methanol-d₄) δ 9.18 (d, J=7.6 Hz, 1H), 8.54 (s, 1H), 7.84 (m, 1H), 7.72 (d, J=7.6 Hz, 1H), 7.68-7.60 (m, 4H), 7.54-7.50 (m, 2H), 7.41 (dd, J=7.6 Hz, J=7.1.2 Hz, 1H), 7.27 (d, J=7.2 Hz, 1H), 4.30 (s, 2H), 4.03 (s, 3H), 3.88 (s, 2H), 3.70-3.66 (m, 4H), 3.40 (m, 4H), 3.30 (m, 2H), 3.15-3.06 (m, 1H), 2.83-2.76 (m, 1H), 2.34-2.21 (m, 2H).

### Example 84: 1-((8-(2,2′-Dichloro-3′-(5-((3-(hydroxymethyl)azetidin-1-yl)methyl)-6-methoxypyridin-2-yl)-[1,1′-biphenyl]-3-yl)-4-oxo-4H-pyrido[1,2-a]pyrimidin-3-yl)methyl)azetidine-3-carboxylic acid

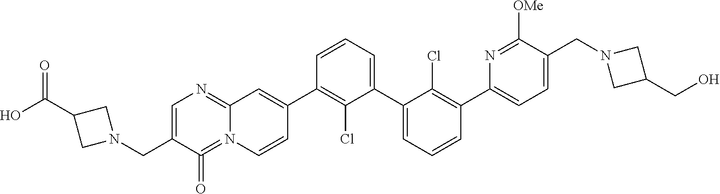

To a mixture of 8-(2,2′-dichloro-3′-(5-((3-(hydroxymethyl)azetidin-1-yl)methyl)-6-methoxypyridin-2-yl)-[1,1′-biphenyl]-3-yl)-4-oxo-4H-pyrido[1,2-a]pyrimidine-3-carbaldehyde (200 mg, 0.33 mmol) and azetidine-3-carboxylic acid (67 mg, 0.67 mmol) in CH₂Cl₂ (5 mL) was added sodium acetate (82 mg, 1 mmol) and 4 Å molecular sieve (150 mg) in one portion under N₂ and the mixture was stirred at r.t. for 2 h. To the reaction mixture was added sodium triacetoxyborohydride (211 mg, 1 mmol). The mixture was stirred at r.t. for 10 h. The mixture was filtered and the filter cake was washed with MeOH (20 mL). The filtrate was concentrated. The residue was purified by reverse phase HPLC to afford 1-((8-(2,2′-dichloro-3′-(5-((3-(hydroxymethyl)azetidin-1-yl)methyl)-6-methoxypyridin-2-yl)-[1,1′-biphenyl]-3-yl)-4-oxo-4H-pyrido[1,2-a]pyrimidin-3-yl)methyl)azetidine-3-carboxylic acid as a white solid (43 mg, 18% yield, m/z: 686 [M+H]⁺). ¹H NMR (400 MHz, Methanol-d₄) δ 9.15 (d, J=7.2 Hz, 1H), 8.48 (s, 1H), 7.82 (m, 1H), 7.73 (d, J=7.2 Hz, 1H), 7.68-7.57 (m, 4H), 7.54-7.50 (m, 2H), 7.42-7.40 (m, 1H), 7.28 (d, J=7.6 Hz, 1H), 4.22 (m, 2H), 4.12-4.02 (m, 7H), 3.94 (s, 2H), 3.76-3.72 (m, 2H), 3.68 (d, J=5.6 Hz, 2H), 3.50-3.46 (m, 2H), 3.42-3.36 (m, 1H), 2.87-2.79 (m, 1H).

### Example 85: 8-(2,2′-Dichloro-3′-(6-methoxy-5-(((((S)-5-oxopyrrolidin-2-yl)methyl)amino)methyl)pyridin-2-yl)-[1,1′-biphenyl]-3-yl)-3-(((((S)-5-oxopyrrolidin-2-yl)methyl)amino)methyl)-4H-quinolizin-4-one

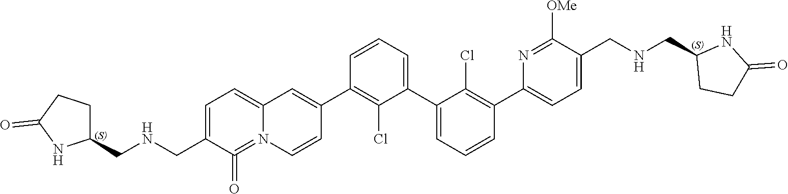

### 8-(3-Bromo-2-chlorophenyl)-4-oxo-4H-quinolizine-3-carbaldehyde

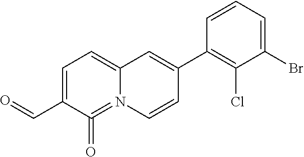

To a solution of 8-chloro-4-oxo-4H-quinolizine-3-carbaldehyde (1 g, 4.82 mmol) and 2-(3-bromo-2-chlorophenyl)-4,4,5,5-tetramethyl-1,3,2-dioxaborolane (1.3 g, 4.1 mmol) in THE (20 mL) and H₂O (4 mL) was added 1,1′-bis(diphenylphosphino)ferrocene-palladium(II) dichloride dichloromethane complex (0.39 g, 0.48 mmol) and potassium carbonate (1.33 g, 9.64 mmol). The mixture was stirred at 80° C. for 1 hr. Water (6 mL) was added to the reaction mixture and the resulting yellow solid was collected by filtration to afford 8-(3-bromo-2-chlorophenyl)-4-oxo-4H-quinolizine-3-carbaldehyde (0.9 g, 51% yield) as a yellow solid. ¹H NMR (400 MHz, DMSO-d₆): δ 10.27 (s, 1H), 9.33 (d, J=7.6 Hz, 1H), 8.16-8.11 (m, 2H), 7.97 (d, J=7.6 Hz, 1H), 7.67-7.61 (m, 2H), 7.48 (t, J=8 Hz, 1H), 7.02 (d, J=7.6 Hz, 1H).

### 8-(2,2′-Dichloro-3′-(5-formyl-6-methoxypyridin-2-yl)-[1,1′-biphenyl]-3-yl)-4-oxo-4H-quinolizine-3-carbaldehyde

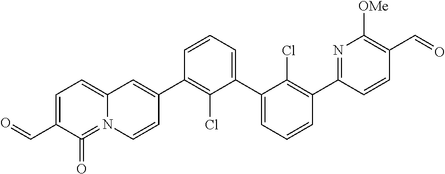

To a solution of 8-(3-bromo-2-chlorophenyl)-4-oxo-4H-quinolizine-3-carbaldehyde (0.5 g, 1.38 mmol) and 6-(2-chloro-3-(4,4,5,5-tetramethyl-1,3,2-dioxaborolan-2-yl)phenyl)-2-methoxynicotinaldehyde (1.14 g, 1.38 mmol) in THE (20 mL) and H₂O (5 mL) was added [1,1-Bis(di-tert-butylphosphino)ferrocene]palladium(II) dichloride (0.09 g, 0.14 mmol) and potassium phosphate (0.59 g, 2.76 mmol). The mixture was stirred at 80° C. for 5 hr. H₂O (10 mL) was added to the mixture and the resulting yellow solid was collected by filtration to give 8-(2,2′-dichloro-3′-(5-formyl-6-methoxypyridin-2-yl)-[1,1′-biphenyl]-3-yl)-4-oxo-4H-quinolizine-3-carbaldehyde (0.5 g, 69% yield) was obtained as a yellow solid. ¹H NMR (400 MHz, DMSO-d₆) δ 10.31 (s, 1H), 10.27 (s, 1H), 9.36 (d, J=7.6 Hz, 1H), 8.24-8.20 (m, 2H), 8.12 (d, J=7.6 Hz, 1H), 7.71-7.49 (m, 8H), 7.05 (d, J=8.4 Hz, 1H), 4.07 (s, 3H).

### 8-(2,2′-Dichloro-3′-(6-methoxy-5-(((((S)-5-oxopyrrolidin-2-yl)methyl)amino)methyl)pyridin-2-yl)-[1,1′-biphenyl]-3-yl)-3-(((((S)-5-oxopyrrolidin-2-yl)methyl)amino)methyl)-4H-quinolizin-4-one

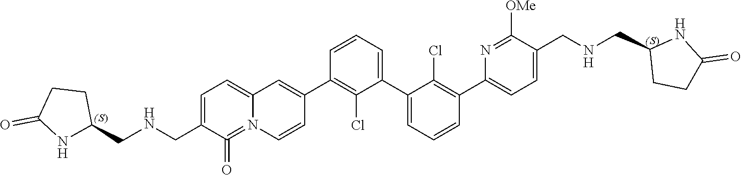

To a solution of 8-(2,2′-dichloro-3′-(5-formyl-6-methoxypyridin-2-yl)-[1,1′-biphenyl]-3-yl)-4-oxo-4H-quinolizine-3-carbaldehyde (250 mg, 0.47 mmol) and (S)-5-(aminomethyl)pyrrolidin-2-one HCl salt (427 mg, 2.83 mmol) in CH₂Cl₂ (10 mL) was added 4 Å molecular sieves (250 mg) and sodium acetate (387 mg, 4.72 mmol). The mixture was stirred at 15° C. for 50 min. Sodium triacetoxyborohydride (601 mg, 2.83 mmol) was added to the mixture and the mixture was stirred at r.t. for 10 min. The reaction mixture combined with another batch at 50 mg scale was filtered and the filtrate was concentrated. The residue was purified by reverse phase HPLC to afford 8-(2,2′-dichloro-3′-(6-methoxy-5-(((((S)-5-oxopyrrolidin-2-yl)methyl)amino)methyl)pyridin-2-yl)-[1,1′-biphenyl]-3-yl)-3-(((((S)-5-oxopyrrolidin-2-yl)methyl)amino)methyl)-4H-quinolizin-4-one as formic acid salt (87.6 mg, 24% yield, m/z: 725 [M+H]⁺) as a yellow solid. ¹H NMR (400 MHz, Methanol-d₄) δ 9.20 (d, J=8 Hz, 1H), 8.44 (s, 1H), 7.96 (d, J=7.6 Hz, 1H), 7.92 (d, J=1.6 Hz, 1H), 7.79 (d, J=7.6 Hz, 1H), 7.69-7.66 (dd, J=8 Hz, J=1.6 Hz, 1H), 7.62-7.41 (m, 6H), 7.29 (d, J=7.2 Hz, 1H), 7.08 (d, J=8 Hz, 1H), 4.29-4.20 (m, 2H), 4.06 (s, 3H), 4.03-3.92 (m, 4H), 3.15-3.04 (m, 2H), 2.92-2.82 (m, 2H), 2.45-2.28 (m, 6H), 1.95-1.82 (m, 2H).

### Example 86: 8-(2,2′-Dichloro-5′-fluoro-3′-(6-methoxy-5-(((((S)-5-oxopyrrolidin-2-yl)methyl)amino)methyl)pyridin-2-yl)-[1,1′-biphenyl]-3-yl)-3-(((((S)-5-oxopyrrolidin-2-yl)methyl)amino)methyl)-4H-pyrido[1,2-a]pyrimidin-4-one

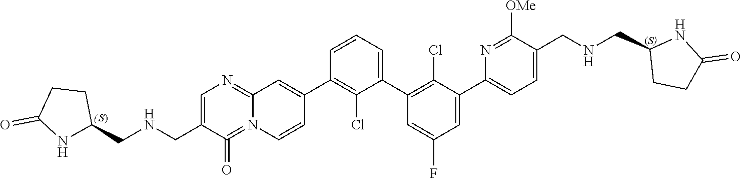

### 8-(3′-Bromo-2,2′-dichloro-5′-fluoro-[1,1′-biphenyl]-3-yl)-4-oxo-4H-pyrido[1,2-a]pyrimidine-3-carbaldehyde

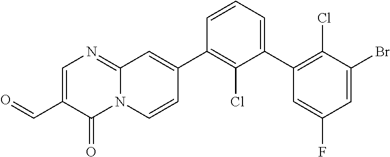 [SMILES: O=Cc1cnc2cc(-c3cccc(-c4cc(F)cc(Br)c4Cl)c3Cl)ccn2c1=O]

To a solution of 8-(2-chloro-3-(4,4,5,5-tetramethyl-1,3,2-dioxaborolan-2-yl)phenyl)-4-oxo-4H-pyrido[1,2-a]pyrimidine-3-carbaldehyde (0.3 g, 0.73 mmol) and 1,3-dibromo-2-chloro-5-fluorobenzene (0.42 g, 1.46 mmol) in dioxane (10 mL) under nitrogen gas was added 1,1′-bis(diphenylphosphino)ferrocene-palladium(ii) dichloride (DCM adduct) (0.04 g, 0.05 mmol) and potassium carbonate (0.25 g, 1.83 mmol). The mixture was stirred at 90° C. for 3 hrs. The mixture was filtered through CELITE® and the filter cake was washed with EtOAc (3×40 mL). The filtrate was washed with saturated aqueous brine solution (2×30 mL), then dried over Na₂SO₄ and concentrated under reduced pressure. The residue was purified by normal phase flash SiO₂ chromatography using a linear gradient (0-40% ethyl acetate hexanes) to afford 8-(3′-bromo-2,2′-dichloro-5′-fluoro-[1,1′-biphenyl]-3-yl)-4-oxo-4H-pyrido[1,2-a]pyrimidine-3-carbaldehyde (0.16 g, 44% yield). MS: m/z found 490 [M+H]⁺; ¹H NMR (400 MHz, Chloroform-d) δ10.41 (s, 1H), 9.35-9.24 (m, 1H), 8.94 (s, 1H), 7.92 (d, J=8.9, 1.9, 0.7 Hz, 1H), 7.62-7.37 (m, 5H), 7.05 (dd, J=8.0, 2.9, 0.7 Hz, 1H).

### 8-(2,2′-Dichloro-5′-fluoro-3′-(5-formyl-6-methoxypyridin-2-yl)-[1,1′-biphenyl]-3-yl)-4-oxo-4H-pyrido[1,2-a]pyrimidine-3-carbaldehyde

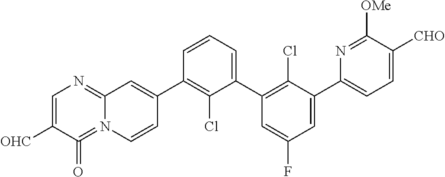

To a solution of 8-(3′-bromo-2,2′-dichloro-5′-fluoro-[1,1′-biphenyl]-3-yl)-4-oxo-4H-pyrido[1,2-a]pyrimidine-3-carbaldehyde (0.07 g, 0.14 mmol) and (5-formyl-6-methoxypyridin-2-yl)boronic acid (0.03 g, 0.18 mmol) in dioxane (10 mL) under nitrogen gas was added 1,1′-bis(diphenylphosphino)ferrocene-palladium(ii) dichloride (DCM adduct) (0.01 g, 0.01 mmol) and potassium carbonate (0.05 g, 0.36 mmol). The mixture was stirred at 90° C. for 3 hrs. The mixture was filtered through CELITE® and the filter cake was washed with EtOAc (3×40 mL). The filtrate was washed with saturated aqueous brine solution (2×30 mL) then dried over Na₂SO₄ and concentrated under reduced pressure. The residue was purified by normal phase flash SiO₂ chromatography using a linear gradient (0-3% MeOH/DCM) to afford 8-(2,2′-dichloro-5′-fluoro-3′-(5-formyl-6-methoxypyridin-2-yl)-[1,1′-biphenyl]-3-yl)-4-oxo-4H-pyrido[1,2-a]pyrimidine-3-carbaldehyde (0.05 g, 64% yield). MS: m/z found 548 [M+H]⁺.

### 8-(2,2′-Dichloro-5′-fluoro-3′-(6-methoxy-5-(((((S)-5-oxopyrrolidin-2-yl)methyl)amino)methyl)pyridin-2-yl)-[1,1′-biphenyl]-3-yl)-3-(((((S)-5-oxopyrrolidin-2-yl)methyl)amino)methyl)-4H-pyrido[1,2-a]pyrimidin-4-one

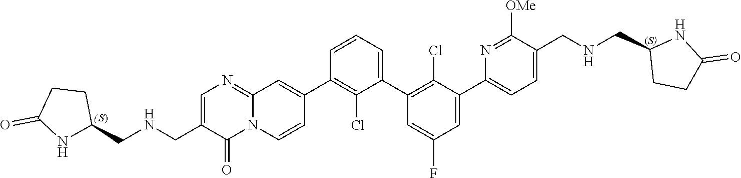

To a mixture of 8-(2,2′-dichloro-5′-fluoro-3′-(5-formyl-6-methoxypyridin-2-yl)-[1,1′-biphenyl]-3-yl)-4-oxo-4H-pyrido[1,2-a]pyrimidine-3-carbaldehyde (0.03 g, 0.05 mmol) and (S)-5-(aminomethyl)pyrrolidin-2-one (0.02 g, 0.16 mmol) in 1:1 THF/MeOH (6 mL) was added acetic acid (0.01 g, 0.14 mmol) and 4 Å molecular sieve (1 g). The mixture was stirred at r.t. for 3 hrs. Sodium cyanoborohydride (0.01 g, 0.16 mmol) was added and the mixture was stirred at r.t. for 1 hr. The reaction was concentrated, and residue purified by Gilson. ACN was evaporated off, then aqueous layer was brought to pH=9 with saturated NaHCO₃, then extracted with 3:1 CHCl₃/IPA (3×40 mL). Combined organic was washed with brine, then dried over Na₂SO₄. Characterization at this point showed an impurity, so material was purified on ISCO using a liner gradient of 0-10% MeOH (containing 1% 7M NH₃ in MeOH) to give 10 mg (30%) of the product as a free base affording 8-(2,2′-dichloro-5′-fluoro-3′-(6-methoxy-5-(((((S)-5-oxopyrrolidin-2-yl)methyl)amino)methyl)pyridin-2-yl)-[1,1′-biphenyl]-3-yl)-3-(((((S)-5-oxopyrrolidin-2-yl)methyl)amino)methyl)-4H-pyrido[1,2-a]pyrimidin-4-one as a white solid. MS: m/z found 744 [M+H]⁺. H NMR (400 MHz, Chloroform-d) δ9.08 (d, J=7.4, 0.7 Hz, 1H), 8.33 (s, 1H), 7.76 (s, 1H), 7.61 (d, J=7.4 Hz, 1H), 7.56-7.40 (m, 4H), 7.37 (dd, J=7.4, 1.9 Hz, 1H), 7.31 (dd, J=7.4, 0.9 Hz, 1H), 7.08 (dd, J=7.9, 3.1 Hz, 1H), 6.22 (s, 1H), 6.08 (s, 1H), 4.03 (d, J=0.6 Hz, 3H), 3.94-3.68 (m, 6H), 2.86-2.76 (m, 2H), 2.62-2.52 (m, 2H), 2.41-2.17 (m, 6H), 1.84-1.68 (m, 2H).

### Example 87: 8-(2,2′-dichloro-4″-(((((S)-5-oxopyrrolidin-2-yl)methyl)amino)methyl)-3″-(trifluoromethoxy)-[1,1′:3′,1″-terphenyl]-3-yl)-3-(((((S)-5-oxopyrrolidin-2-yl)methyl)amino)methyl)-4H-pyrido[1,2-a]pyrimidin-4-one

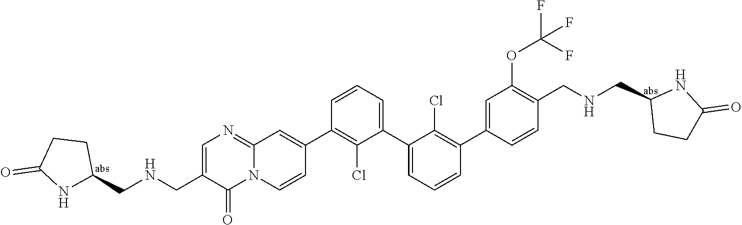

### 8-[2-chloro-3-[2-chloro-3-[4-formyl-3-(trifluoromethoxy)phenyl]phenyl]phenyl]-4-oxo-pyrido[1,2-a]pyrimidine-3-carbaldehyde

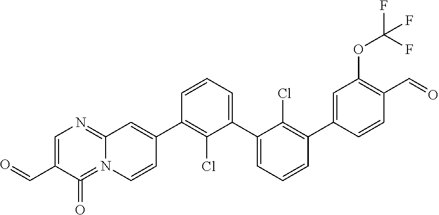

To a solution of 8-[2-chloro-3-[2-chloro-3-(4,4,5,5-tetramethyl-1,3,2-dioxaborolan-2-yl)phenyl]phenyl]-4-oxo-pyrido[1,2-a]pyrimidine-3-carbaldehyde (70 mg, 0.13 mmol), 4-bromo-2-(trifluoromethoxy)benzaldehyde (54 mg, 0.20 mmol) and potassium carbonate (56 mg, 0.40 mmol) in degassed dioxane/water (6:1) was added Pd(PPh₃)₄ (16 mg, 0.01 mmol) and the mixture was stirred at 90° C. for overnight under N₂(g). Water was added and the mixture was extracted into EtOAc three times. The combined organic layers were dried over anhydrous sodium sulfate, concentrated, and purified on silica gel column using EtOAc in hexanes (0 to 60% gradient) to give 8-[2-chloro-3-[2-chloro-3-[4-formyl-3-(trifluoromethoxy)phenyl]phenyl]phenyl]-4-oxo-pyrido[1,2-a]pyrimidine-3-carbaldehyde (38 mg, 48.5%). MS: m/z found 583.1 [M+H]⁺.

### 8-[2-Chloro-3-[2-chloro-3-[4-[[[(2S)-5-oxopyrrolidin-2-yl]methylamino]methyl]-3-(trifluoromethoxy)phenyl]phenyl]phenyl]-3-[[[(2S)-5-oxopyrrolidin-2-yl]methylamino]methyl]pyrido[1,2-a]pyrimidin-4-one

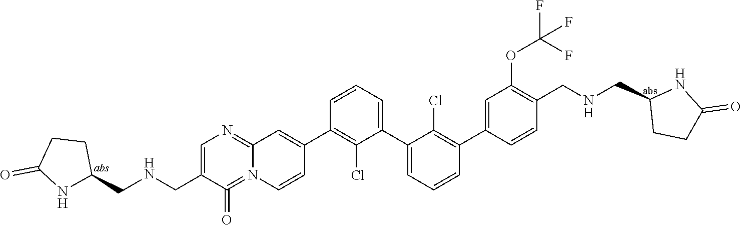

A mixture of 8-[2-chloro-3-[2-chloro-3-[4-formyl-3-(trifluoromethoxy)phenyl]phenyl]phenyl]-4-oxo-pyrido[1,2-a]pyrimidine-3-carbaldehyde (35 mg, 0.06 mmol), (5S)-5-(aminomethyl)pyrrolidin-2-one (27 mg, 0.24 mmol) and acetic acid (7 uL, 7 mg, 0.12 mmol) in MeOH/THF (1:1) was stirred for 1.5 hour at rt. NaBH₃CN (15.08 mg, 0.24 mmol) was added, the mixture was stirred for additional 5 h, quenched with water and directly purified by HPLC. The collected fraction was neutralized with sat. NaHCO₃ (aq.), acetonitrile was evaporated, and aqueous phase was extracted into DCM three times. The combined organic extracts were dried over anhydrous magnesium sulfate and concentrated in vacuo to give 8-[2-chloro-3-[2-chloro-3-[4-[[[(2S)-5-oxopyrrolidin-2-yl]methylamino]methyl]-3-(trifluoromethoxy)phenyl]phenyl]phenyl]-3-[[[(2S)-5-oxopyrrolidin-2-yl]methylamino]methyl]pyrido[1,2-a]pyrimidin-4-one (26 mg, 55.6%). MS: m/z found 779.2 [M+H]⁺; ¹H NMR (400 MHz, Methanol-d₄): δ 9.14 (dd, J=7.4, 0.7 Hz, 1H), 8.42 (s, 1H), 7.77 (dd, J=1.9, 0.7 Hz, 1H), 7.68 (d, J=8.0 Hz, 1H), 7.65-7.58 (m, 2H), 7.55-7.50 (m, 3H), 7.49-7.45 (m, 2H), 7.42-7.38 (m, 2H), 3.94 (d, J=2.6 Hz, 2H), 3.90-3.79 (m, 4H), 2.80-2.63 (m, 4H), 2.38-2.24 (m, 6H), 1.88-1.77 (m, 2H).

### Example 88: 8-(2′-Chloro-2-methyl-4″-(((((S)-5-oxopyrrolidin-2-yl)methyl)amino)methyl)-3″-(trifluoromethoxy)-[1,1′:3′,1″-terphenyl]-3-yl)-3-(((((S)-5-oxopyrrolidin-2-yl)methyl)amino)methyl)-4H-pyrido[1,2-a]pyrimidin-4-one

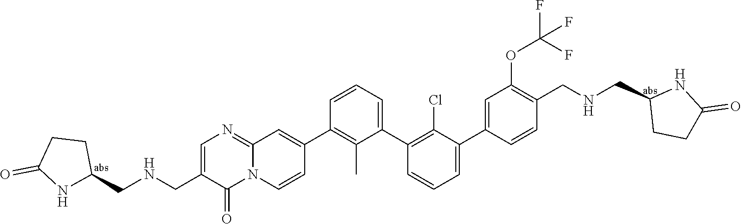

### 8-[3-[2-Chloro-3-[4-formyl-3-(trifluoromethoxy)phenyl]phenyl]-2-methyl-phenyl]-4-oxo-pyrido[1,2-a]pyrimidine-3-carbaldehyde

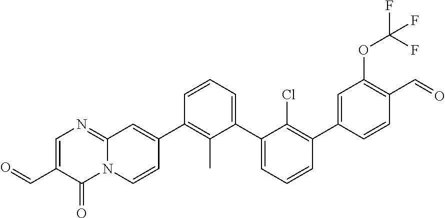 [SMILES: Cc1c(-c2ccn3c(=O)c(C=O)cnc3c2)cccc1-c1cccc(-c2ccc(C=O)c(OC(F)(F)F)c2)c1Cl]

To a solution of 8-[3-[2-chloro-3-(4,4,5,5-tetramethyl-1,3,2-dioxaborolan-2-yl)phenyl]-2-methyl-phenyl]-4-oxo-pyrido[1,2-a]pyrimidine-3-carbaldehyde (60 mg, 0.12 mmol), 4-bromo-2-(trifluoromethoxy)benzaldehyde (48 mg, 0.18 mmol) and potassium carbonate (50 mg, 0.36 mmol) in degassed dioxane/water (6:1) was added Pd(PPh₃)₄ (14 mg, 0.01 mmol) and the mixture was stirred at 90° C. for 4 hours under N₂(g). Water was added and the mixture was extracted into EtOAc three times. The combined organic layers were dried over anhydrous sodium sulfate, concentrated, and purified on silica gel column using EtOAc in hexanes (0 to 60% gradient) to give 8-[3-[2-chloro-3-[4-formyl-3-(trifluoromethoxy)phenyl]phenyl]-2-methyl-phenyl]-4-oxo-pyrido[1,2-a]pyrimidine-3-carbaldehyde (30 mg, 44.5%). MS: m/z found 563.2 [M+H]^{m}.

### 8-[3-[2-Chloro-3-[4-[[[(2S)-5-oxopyrrolidin-2-yl]methylamino]methyl]-3-(trifluoromethoxy)phenyl]phenyl]-2-methyl-phenyl]-3-[[[(2S)-5-oxopyrrolidin-2-yl]methylamino]methyl]pyrido[1,2-a]pyrimidin-4-one

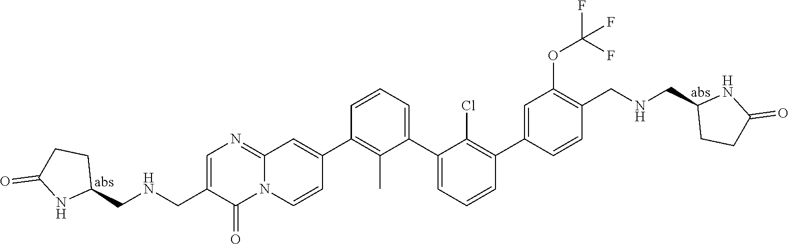 [SMILES: Cc1c(-c2ccn3c(=O)c(CNC[C@@H]4CCC(=O)N4)cnc3c2)cccc1-c1cccc(-c2ccc(CNC[C@@H]3CCC(=O)N3)c(OC(F)(F)F)c2)c1Cl]

A mixture of 8-[3-[2-chloro-3-[4-formyl-3-(trifluoromethoxy)phenyl]phenyl]-2-methyl-phenyl]-4-oxo-pyrido[1,2-a]pyrimidine-3-carbaldehyde (30 mg, 0.05 mmol), (5S)-5-(aminomethyl)pyrrolidin-2-one (24 mg, 0.21 mmol) and acetic acid (6 uL, 6 mg, 0.11 mmol) in MeOH/THF (1:1) was stirred for 1.5 hour at rt. NaBH₃CN (13 mg, 0.21 mmol) was added, the mixture was stirred for additional 5 h, quenched with water and directly purified by HPLC. The collected fraction was neutralized with sat. NaHCO₃(aq.), acetonitrile was evaporated, and aqueous phase was extracted into DCM three times. The combined organic extracts were dried over anhydrous magnesium sulfate and concentrated in vacuo to give 8-[3-[2-chloro-3-[4-[[[(2S)-5-oxopyrrolidin-2-yl]methylamino]methyl]-3-(trifluoromethoxy)phenyl]phenyl]-2-methyl-phenyl]-3-[[[(2S)-5-oxopyrrolidin-2-yl]methylamino]methyl]pyrido[1,2-a]pyrimidin-4-one (24 mg, 59.3%). MS: m/z found 759.2 [M+H]⁺; ¹H NMR (400 MHz, Methanol-d₄): δ 9.13 (dd, J=7.4, 0.7 Hz, 1H), 8.40 (s, 1H), 7.67 (d, J=8.0 Hz, 1H), 7.65 (dd, J=1.9, 0.7 Hz, 1H), 7.51 (t, J=7.6 Hz, 1H), 7.48-7.41 (m, 5H), 7.41-7.38 (m, 1H), 7.37 (dd, J=7.4, 1.8 Hz, 1H), 7.32 (dd, J=6.1, 2.9 Hz, 1H), 4.01-3.76 (m, 6H), 2.84-2.62 (m, 4H), 2.42-2.21 (m, 6H), 2.12 (s, 3H), 1.90-1.75 (m, 2H).

### Example 89: 8-(2,2′-Dichloro-3′-(6-(methylamino)-5-(((((S)-5-oxopyrrolidin-2-yl)methyl)amino)methyl)pyridin-2-yl)-[1,1′-biphenyl]-3-yl)-3-(((((S)-5-oxopyrrolidin-2-yl)methyl)amino)methyl)-4H-pyrido[1,2-a]pyrimidin-4-one

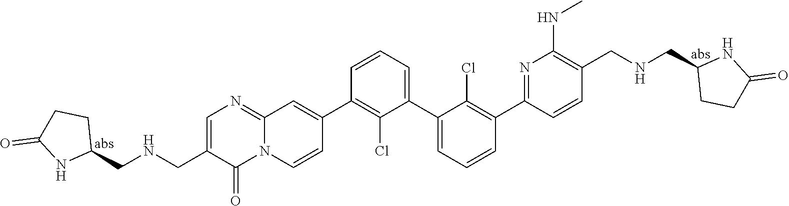 [SMILES: CNc1nc(-c2cccc(-c3cccc(-c4ccn5c(=O)c(CNC[C@@H]6CCC(=O)N6)cnc5c4)c3Cl)c2Cl)ccc1CNC[C@@H]1CCC(=O)N1]

### 8-[2-Chloro-3-[2-chloro-3-[5-formyl-6-(methylamino)-2-pyridyl]phenyl]phenyl]-4-oxo-pyrido[1,2-a]pyrimidine-3-carbaldehyde

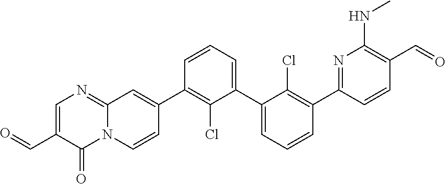 [SMILES: CNc1nc(-c2cccc(-c3cccc(-c4ccn5c(=O)c(C=O)cnc5c4)c3Cl)c2Cl)ccc1C=O]

To a solution of 8-[2-chloro-3-[2-chloro-3-(4,4,5,5-tetramethyl-1,3,2-dioxaborolan-2-yl)phenyl]phenyl]-4-oxo-pyrido[1,2-a]pyrimidine-3-carbaldehyde (50 mg, 0.10 mmol), 6-bromo-2-(methylamino)pyridine-3-carbaldehyde (31 mg, 0.14 mmol) and potassium carbonate (40 mg, 0.29 mmol) in degassed dioxane/water (6:1) was added Pd(PPh₃)₄ (11 mg, 0.01 mmol) and the mixture was stirred at 90° C. for 4 hours under N₂(g). Water was added and the mixture was extracted into EtOAc three times. The combined organic layers were dried over anhydrous sodium sulfate, concentrated, and purified on silica gel column using EtOAc in hexanes (0 to 60% gradient). MS: m/z found 529.2 [M+H]⁺.

### 8-[2-Chloro-3-[2-chloro-3-[6-(methylamino)-5-[[[(2S)-5-oxopyrrolidin-2-yl]methylamino]methyl]-2-pyridyl]phenyl]phenyl]-3-[[[(2S)-5-oxopyrrolidin-2-yl]methylamino]methyl]pyrido[1,2-a]pyrimidin-4-one

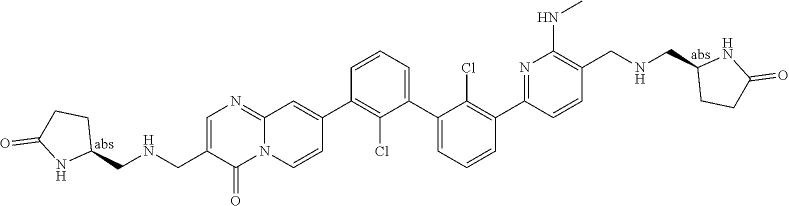 [SMILES: CNc1nc(-c2cccc(-c3cccc(-c4ccn5c(=O)c(CNC[C@@H]6CCC(=O)N6)cnc5c4)c3Cl)c2Cl)ccc1CNC[C@@H]1CCC(=O)N1]

A mixture of 8-[2-chloro-3-[2-chloro-3-[5-formyl-6-(methylamino)-2-pyridyl]phenyl]phenyl]-4-oxo-pyrido[1,2-a]pyrimidine-3-carbaldehyde (24 mg, 0.05 mmol), (5S)-5-(aminomethyl)pyrrolidin-2-one (21 mg, 0.18 mmol) and acetic acid (5 uL, 5 mg, 0.09 mmol) in MeOH/THF (1:1) was stirred for 1.5 hour at rt. NaBH₃CN (11 mg, 0.18 mmol) was added, the mixture was stirred for additional 5 h, quenched with water and directly purified by HPLC. The collected fraction was neutralized with sat. NaHCO₃(aq.), acetonitrile was evaporated, and aqueous phase was extracted into DCM three times. The combined organic extracts were dried over anhydrous magnesium sulfate and concentrated in vacuo to give 8-[2-chloro-3-[2-chloro-3-[6-(methylamino)-5-[[[(2S)-5-oxopyrrolidin-2-yl]methylamino]methyl]-2-pyridyl]phenyl]phenyl]-3-[[[(2S)-5-oxopyrrolidin-2-yl]methylamino]methyl]pyrido[1,2-a]pyrimidin-4-one (18 mg, 54.7%). MS: m/z found 725.2 [M+H]⁺; ¹H NMR (400 MHz, Methanol-d₄): δ 9.12 (dd, J=7.4, 0.7 Hz, 1H), 8.41 (s, 1H), 7.76 (dd, J=1.9, 0.8 Hz, 1H), 7.63-7.56 (m, 3H), 7.53 (dd, J=7.4, 1.9 Hz, 1H), 7.49 (dd, J=4.9, 2.2 Hz, 1H), 7.48-7.44 (m, 1H), 7.38 (d, J=7.4 Hz, 1H), 7.35 (dd, J=7.6, 1.7 Hz, 1H), 6.76 (d, J=7.3 Hz, 1H), 3.87 (d, J=4.1 Hz, 2H), 3.86-3.79 (m, 2H), 3.77 (d, J=3.6 Hz, 2H), 2.98 (s, 3H), 2.80-2.64 (m, 4H), 2.37-2.21 (m, 6H), 1.87-1.76 (m, 2H).

### Example 90: 8-(2,2′-Dichloro-3″-methoxy-4″-(((((S)-5-oxopyrrolidin-2-yl)methyl)amino)methyl)-[1,1′:3′,1″-terphenyl]-3-yl)-3-(((((S)-5-oxopyrrolidin-2-yl)methyl)amino)methyl)-4H-pyrido[1,2-a]pyrimidin-4-one

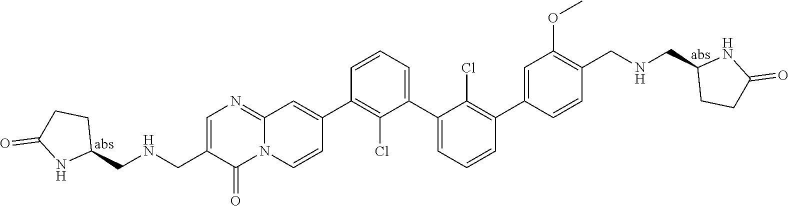 [SMILES: COc1cc(-c2cccc(-c3cccc(-c4ccn5c(=O)c(CNC[C@@H]6CCC(=O)N6)cnc5c4)c3Cl)c2Cl)ccc1CNC[C@@H]1CCC(=O)N1]

### 8-[2-Chloro-3-[2-chloro-3-(4-formyl-3-methoxy-phenyl)phenyl]phenyl]-4-oxo-pyrido[1,2-a]pyrimidine-3-carbaldehyde

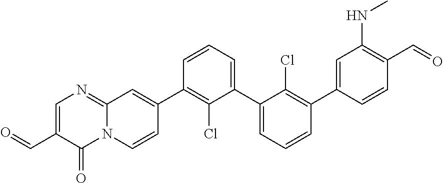 [SMILES: CNc1cc(-c2cccc(-c3cccc(-c4ccn5c(=O)c(C=O)cnc5c4)c3Cl)c2Cl)ccc1C=O]

To a solution of 8-[2-chloro-3-[2-chloro-3-(4,4,5,5-tetramethyl-1,3,2-dioxaborolan-2-yl)phenyl]phenyl]-4-oxo-pyrido[1,2-a]pyrimidine-3-carbaldehyde (70 mg, 0.13 mmol), 4-bromo-2-methoxy-benzaldehyde (43 mg, 0.20 mmol) and potassium carbonate (56 mg, 0.40 mmol) in degassed dioxane/water (6:1) was added Pd(PPh₃)₄ (16 mg, 0.01 mmol) and the mixture was stirred at 90° C. for overnight under N₂(g). Water was added and the mixture was extracted into EtOAc three times. The combined organic layers were dried over anhydrous sodium sulfate, concentrated, and purified on silica gel column using EtOAc in hexanes (0 to 60% gradient) to give 8-[2-chloro-3-[2-chloro-3-(4-formyl-3-methoxy-phenyl)phenyl]phenyl]-4-oxo-pyrido[1,2-a]pyrimidine-3-carbaldehyde (33 mg, 46.4%). MS: m/z found 529.1 [M+H]⁺.

### 8-[2-Chloro-3-[2-chloro-3-[3-methoxy-4-[[[(2S)-5-oxopyrrolidin-2-yl]methylamino]methyl]phenyl]phenyl]phenyl]-3-[[[(2S)-5-oxopyrrolidin-2-yl]methylamino]methyl]pyrido[1,2-a]pyrimidin-4-one

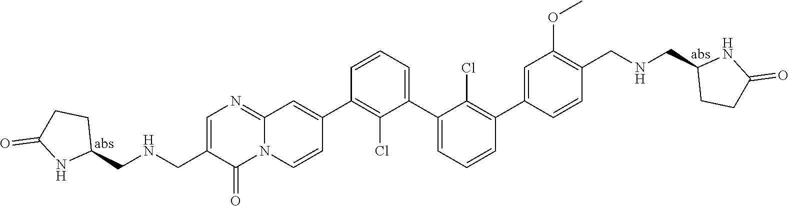 [SMILES: COc1cc(-c2cccc(-c3cccc(-c4ccn5c(=O)c(CNC[C@@H]6CCC(=O)N6)cnc5c4)c3Cl)c2Cl)ccc1CNC[C@@H]1CCC(=O)N1]

A mixture of 8-[2-chloro-3-[2-chloro-3-(4-formyl-3-methoxy-phenyl)phenyl]phenyl]-4-oxo-pyrido[1,2-a]pyrimidine-3-carbaldehyde (33 mg, 0.06 mmol), (5S)-5-(aminomethyl)pyrrolidin-2-one (28 mg, 0.25 mmol) and acetic acid (7 uL, 7 mg, 0.12 mmol) in MeOH/THF (1:1) was stirred for 1.5 hour at rt. NaBH₃CN (16 mg, 0.25 mmol) was added, the mixture was stirred for additional 5 h, quenched with water and directly purified by HPLC. The collected fraction was neutralized with sat. NaHCO₃(aq.), acetonitrile was evaporated, and aqueous phase was extracted into DCM three times. The combined organic extracts were dried over anhydrous magnesium sulfate and concentrated in vacuo to give 8-[2-chloro-3-[2-chloro-3-[3-methoxy-4-[[[(2S)-5-oxopyrrolidin-2-yl]methylamino]methyl]phenyl]phenyl]phenyl]-3-[[[(2S)-5-oxopyrrolidin-2-yl]methylamino]methyl]pyrido[1,2-a]pyrimidin-4-one (25 mg, 55.3%). MS: m/z found 725.2 [M+H]⁺; ¹H NMR (400 MHz, Methanol-d₄): δ 9.14 (dd, J=7.4, 0.8 Hz, 1H), 8.41 (s, 1H), 7.77 (dd, J=1.9, 0.7 Hz, 1H), 7.64-7.57 (m, 2H), 7.55-7.45 (m, 4H), 7.38-7.33 (m, 2H), 7.06 (d, J=1.6 Hz, 1H), 7.02 (dd, J=7.6, 1.6 Hz, 1H), 3.90 (s, 3H), 3.88-3.82 (m, 6H), 2.80-2.61 (m, 4H), 2.40-2.22 (m, 6H), 1.89-1.73 (m, 2H).

### Example 91: (5S)-5-((((6-(2,2′-Dichloro-3′-(5-(((5-fluoropyridin-3-yl)methyl)amino)-8-methyl-5,6,7,8-tetrahydro-1,8-naphthyridin-2-yl)-[1,1′-biphenyl]-3-yl)-2-methoxypyridin-3-yl)methyl)amino)methyl)pyrrolidin-2-one

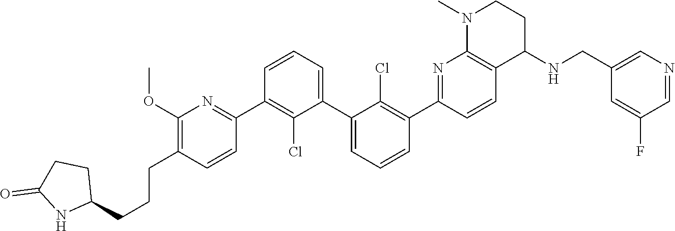 [SMILES: COc1nc(-c2cccc(-c3cccc(-c4ccc5c(n4)N(C)CCC5NCc4cncc(F)c4)c3Cl)c2Cl)ccc1CCC[C@@H]1CCC(=O)N1]

### 7-Bromo-N-((5-fluoropyridin-3-yl)-methyl)-1-methyl-1,2,3,4-tetrahydro-1,8-naphthyridin-4-amine

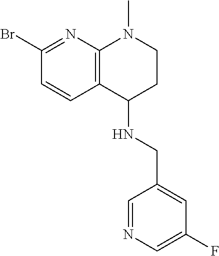 [SMILES: CN1CCC(NCc2cncc(F)c2)c2ccc(Br)nc21]

7-bromo-1-methyl-2,3-dihydro-1,8-naphthyridin-4-one (150 mg, 0.62 mmol), (5-fluoro-3-pyridyl)-methanamine (157 mg, 1.24 mmol) and tetraethoxytitanium (426 mg, 1.87 mmol) were suspended in 4 ml anhydrous THE and the mixture was heated at 105° C. for 5 hours in a sealed tube. The reaction was then cooled to −50° C., and NaBH₄, (71 mg, 1.87 mmol) was added portion-wise under a nitrogen stream. The reaction was warmed to rt over 1 hour and stirred for an additional 10 minutes. The mixture was then added dropwise to an ice water solution (20 mL), resulting in precipitate formation. The solution was filtered through CELITE® and the filtrate was extracted with EtOAc (3×10 mL). The combined organics were washed with brine, dried over sodium sulfate, filtered and concentrated to afford 7-bromo-N-[(5-fluoro-3-pyridyl)methyl]-1-methyl-3,4-dihydro-2H-1,8-naphthyridin-4-amine (144 mg) as a yellow foam. Material was used as—is for the next step. MS: m/z found 351, 353 [M+H]⁺.

### tert-Butyl (7-bromo-1-methyl-1,2,3,4-tetrahydro-1,8-naphthyridin-4-yl)-((5-fluoropyridin-3-yl)methyl)carbamate

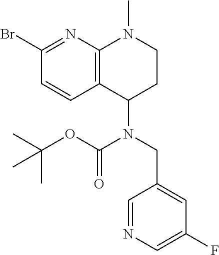 [SMILES: CN1CCC(N(Cc2cncc(F)c2)C(=O)OC(C)(C)C)c2ccc(Br)nc21]

7-Bromo-N-[(5-fluoro-3-pyridyl)methyl]-1-methyl-3,4-dihydro-2H-1,8-naphthyridin-4-amine (144 mg, 0.41 mmol), DMAP (10 mg) and DIEA (285 μL, 212 mg, 1.64 mmol) were dissolved in 4 ml anhydrous THE and tert-butoxycarbonyl tert-butyl carbonate (179 mg, 0.82 mmol) was added portion-wise. The resulting mixture was stirred at rt for 12 hours. EtOAc (10 mL) was added to the reaction and the solution was washed with aqueous saturated sodium bicarbonate solution (5 mL×2) and brine (5 mL×2). The organic layer was concentrated in vacuo to give crude sample, which was purified by silica gel chromatography, eluting with 0-5% MeOH gradient in DCM to afford tert-butyl N-(7-bromo-1-methyl-3,4-dihydro-2H-1,8-naphthyridin-4-yl)-N-[(5-fluoro-3-pyridyl)methyl]carbamate (30 mg, 16.2% yield) as a yellow oil. MS: m/z found 451.1, 453.1 [M+H]⁺.

### (5S)-5-((((6-(2,2′-Dichloro-3′-(5-(((5-fluoropyridin-3-yl)methyl)amino)-8-methyl-5,6,7,8-tetrahydro-1,8-naphthyridin-2-yl)-[1,1′-biphenyl]-3-yl)-2-methoxypyridin-3-yl)methyl)amino)methyl)pyrrolidin-2-one

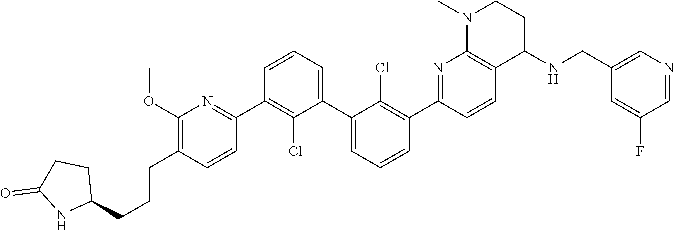 [SMILES: COc1nc(-c2cccc(-c3cccc(-c4ccc5c(n4)N(C)CCC5NCc4cncc(F)c4)c3Cl)c2Cl)ccc1CCC[C@@H]1CCC(=O)N1]

Pd(PPh₃)₄ (15 mg, 0.01 mmol), potassium carbonate (27 mg, 0.19 mmol), 2-[2-chloro-3-[2-chloro-3-(4,4,5,5-tetramethyl-1,3,2-dioxaborolan-2-yl)phenyl]phenyl]-4,4,5,5-tetramethyl-1,3,2-dioxaborolane (31 mg, 0.06 mmol) and tert-butyl N-[(6-chloro-2-methoxy-3-pyridyl)methyl]-N-[[(2S)-5-oxopyrrolidin-2-yl]methyl]carbamate (29 mg, 0.08 mmol) were suspended in 1 ml of 4:1 dioxane/water solvent mixture, and the solution was heated at 105° C. for 30 minutes. The reaction was cooled to rt, filtered through CELITE®, and the filtrate was concentrated. Crude product was purified by reverse phase HPLC to afford tert-butyl N-[[6-[3-[3-[5-[tert-butoxycarbonyl-[(5-fluoro-3-pyridyl)methyl]amino]-8-methyl-6,7-dihydro-5H-1,8-naphthyridin-2-yl]-2-chloro-phenyl]-2-chloro-phenyl]-2-methoxy-3-pyridyl]methyl]-N-[[(2S)-5-oxopyrrolidin-2-yl]methyl]carbamate (7.5 mg, 12.6% yield) as a clear film (racemic mixture of diastereomers). MS: m/z found 927, 929 [M+H]⁺.

tert-butyl N-[[6-[3-[3-[5-[tert-butoxycarbonyl-[(5-fluoro-3-pyridyl)methyl]amino]-8-methyl-6,7-dihydro-5H-1,8-naphthyridin-2-yl]-2-chloro-phenyl]-2-chloro-phenyl]-2-methoxy-3-pyridyl]methyl]-N-[[(2S)-5-oxopyrrolidin-2-yl]methyl]carbamate (17.00 mg, 0.02 mmol) was dissolved in 0.5 ml DCM and 4M HCl solution in dioxane (23 μL, 0.09 mmol) was added. The reaction was stirred at rt for 20 minutes and the mixture was subsequently concentrated. Crude sample was purified by reverse phase HPLC to afford (5S)-5-[[[6-[2-chloro-3-[2-chloro-3-[5-[(5-fluoro-3-pyridyl)methylamino]-8-methyl-6,7-dihydro-5H-1,8-naphthyridin-2-yl]phenyl]phenyl]-2-methoxy-3-pyridyl]methylamino]methyl]pyrrolidin-2-one (1.9 mg, 14.1% yield) as a clear film. MS: m/z found 726.45, 728.45 [M+H]⁺. ¹H NMR (400 MHz, Chloroform-d) δ 8.43 (s, 1H), 8.37 (d, J=2.8 Hz, 1H), 7.65 (dt, J=7.7, 1.7 Hz, 2H), 7.55 (dd, J=15.2, 8.3 Hz, 2H), 7.44-7.41 (m, 1H), 7.40-7.35 (m, 2H), 7.35-7.31 (m, 1H), 7.30 (s, 1H), 7.28 (d, J=2.7 Hz, 1H), 6.89 (dd, J=7.5, 1.9 Hz, 1H), 5.94 (s, 1H), 4.03 (s, 3H), 3.96 (d, J=5.1 Hz, 2H), 3.80 (s, 3H), 3.76 (s, 1H), 3.65-3.54 (m, 1H), 3.39-3.30 (m, 1H), 3.20 (s, 3H), 2.79 (dd, J=12.0, 4.2 Hz, 1H), 2.56 (dd, J=12.0, 8.6 Hz, 1H), 2.36 (t, J=8.1 Hz, 2H), 2.30-2.16 (m, 1H), 2.10-1.98 (m, 2H), 1.82-1.73 (m, 1H). (racemic compound).

### Example 92: (S)-1-((8-(2,2′-Dichloro-3′-(5-((3-(hydroxymethyl)azetidin-1-yl)methyl)-6-methoxypyrazin-2-yl)-[1,1′-biphenyl]-3-yl)-4-oxo-4H-pyrido[1,2-a]pyrimidin-3-yl)methyl)pyrrolidine-3-carboxylic acid

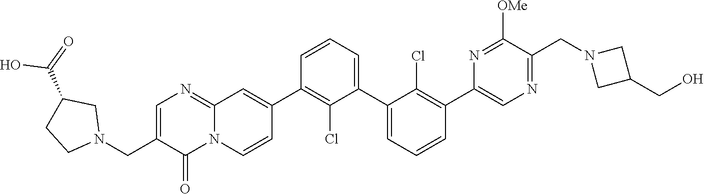 [SMILES: COc1nc(-c2cccc(-c3cccc(-c4ccn5c(=O)c(CN6CC[C@H](C(=O)O)C6)cnc5c4)c3Cl)c2Cl)cnc1CN1CC(CO)C1]

### 5-(3-Bromo-2-chlorophenyl)-3-methoxypyrazine-2-carbaldehyde6-(3-bromo-2-chlor

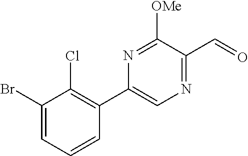 [SMILES: COc1nc(-c2cccc(Br)c2Cl)cnc1C=O]

To a mixture of 2-(3-bromo-2-chlorophenyl)-4,4,5,5-tetramethyl-1,3,2-dioxaborolane (14.3 g, 45 mmol) and 5-bromo-3-methoxypyrazine-2-carbaldehyde (9.8 g, 45 mmol) in DME (100 mL) and H₂O (20 mL) was added sodium carbonate (14.3 g, 135 mmol) and tetrakis(triphenylphosphine)palladium (5.22 g, 4.52 mmol). The mixture was stirred at 80° C. for 3 h under N₂. The mixture was concentrated. The residue was triturated with ethyl acetate (100 mL) at r.t. for 30 min. The mixture was filtered to afford the filter cake. The filter cake was triturated with H₂O (250 mL) at r.t. for 30 min. The mixture was filtered. The filter cake was concentrated to afford 5-(3-bromo-2-chlorophenyl)-3-methoxypyrazine-2-carbaldehyde as a white solid (9.5 g, 55% yield). ¹H NMR (400 MHz, DMSO-d₆): δ 10.17 (s, 1H), 8.78 (s, 1H), 7.98 (dd, J=8 Hz, J=1.6 Hz, 1H), 7.73 (dd, J=7.6 Hz, J=1.6 Hz, 1H), 7.48 (t, J=8 Hz, 1H), 4.06 (s, 3H).

### (1-((5-(3-Bromo-2-chlorophenyl)-3-methoxypyrazin-2-yl)methyl)azetidin-3-yl)methanol

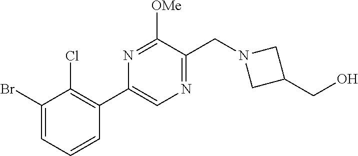 [SMILES: COc1nc(-c2cccc(Br)c2Cl)cnc1CN1CC(CO)C1]

To a mixture of 5-(3-bromo-2-chlorophenyl)-3-methoxypyrazine-2-carbaldehyde (2.5 g, 7.63 mmol) and azetidin-3-ylmethanol as hydrochloride salt (1.4 g, 11.5 mmol) in CH₂Cl₂ (120 mL) was added sodium acetate (1.25 g, 15.3 mmol) and sodium triacetoxyborohydride (3.24 g, 15.3 mmol,) in one portion under N₂. The mixture was stirred at r.t. for 12 h. The mixture was concentrated. The residue was purified by normal phase SiO₂ chromatography (60-100% ethyl acetate/petroleum ether to ethyl acetate/THF/MeOH=2/1/1) to afford (1-((5-(3-bromo-2-chlorophenyl)-3-methoxypyrazin-2-yl)methyl)azetidin-3-yl)methanol as a light yellow gum (2.5 g, 82% yield). ¹H NMR (400 MHz, DMSO-d₆): δ 8.40 (s, 1H), 7.91 (dd, J=8 Hz, J=1.6 Hz, 1H), 7.64 (dd, J=8 Hz, J=1.6 Hz, 1H), 7.43 (t, J=8 Hz, 1H), 3.95 (s, 3H), 3.88 (s, 2H), 3.52-3.49 (m, 4H), 3.26-3.17 (m, 2H), 2.59-2.54 (m, 1H).

### 8-(2,2′-Dichloro-3′-(5-((3-(hydroxymethyl)azetidin-1-yl)methyl)-6-methoxypyrazin-2-yl)-[1,1′-biphenyl]-3-yl)-4-oxo-4H-pyrido[1,2-a]pyrimidine-3-carbaldehyde

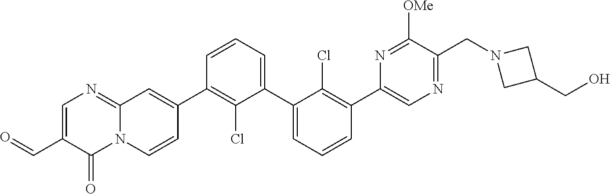 [SMILES: COc1nc(-c2cccc(-c3cccc(-c4ccn5c(=O)c(C=O)cnc5c4)c3Cl)c2Cl)cnc1CN1CC(CO)C1]

To a mixture of (1-((5-(3-bromo-2-chlorophenyl)-3-methoxypyrazin-2-yl)methyl)azetidin-3-yl)methanol (0.46 g, 1.15 mmol) and 8-(2-chloro-3-(4,4,5,5-tetramethyl-1,3,2-dioxaborolan-2-yl)phenyl)-4-oxo-4H-pyrido[1,2-a]pyrimidine-3-carbaldehyde (0.61 g, 1.5 mmol) in dioxane/H₂O mixture (6:1, 14 mL) was added potassium phosphate (0.98 g, 4.62 mmol) and [1,1′-bis(di-tert-butylphosphino)ferrocene]dichloropalladium(II) (0.07 g, 0.11 mmol) in one portion under N₂. The mixture was stirred at 110° C. for 0.5 h. The mixture was concentrated to give the residue. To the residue was added water (50 mL) and saturated aqueous brine solution (50 mL). The mixture was extracted with ethyl acetate/THF mixture (1:1, 100 mL). The organic phase was dried with anhydrous sodium sulfate, filtered and concentrated. The residue was purified by normal phase SiO₂ chromatography (60-100% ethyl acetate/petroleum ether to ethyl acetate/THF/MeOH=2/1/1) to afford 8-(2,2′-dichloro-3′-(5-((3-(hydroxymethyl)azetidin-1-yl)methyl)-6-methoxypyrazin-2-yl)-[1,1′-biphenyl]-3-yl)-4-oxo-4H-pyrido[1,2-a]pyrimidine-3-carbaldehyde as a yellow solid (0.4 g, 51% yield, m/z: 602 [M+H]⁺). ¹H NMR (400 MHz, DMSO-d₆): δ 10.26 (s, 1H), 9.37 (d, J=7.2 Hz, 1H), 8.87 (s, 1H), 8.46 (s, 1H), 7.99 (d, J=1.2 Hz, 1H), 7.78 (dd, J=7.2 Hz, J=2 Hz, 1H), 7.74-7.65 (m, 3H), 7.63-7.56 (m, 2H), 7.52-7.49 (m, 1H), 4.30 (s, 2H), 4.10 (s, 3H), 4.03-3.99 (m, 2H), 3.77 (t, J=8 Hz, 2H), 3.71 (d, J=5.2 Hz, 2H), 2.95-2.91 (m, 1H).

### (S)-1-((8-(2,2′-Dichloro-3′-(5-((3-(hydroxymethyl)azetidin-1-yl)methyl)-6-methoxypyrazin-2-yl)-[1,1′-biphenyl]-3-yl)-4-oxo-4H-pyrido[1,2-a]pyrimidin-3-yl)methyl)pyrrolidine-3-carboxylic acid

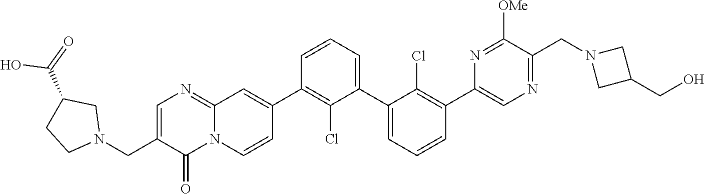 [SMILES: COc1nc(-c2cccc(-c3cccc(-c4ccn5c(=O)c(CN6CC[C@H](C(=O)O)C6)cnc5c4)c3Cl)c2Cl)cnc1CN1CC(CO)C1]

To a mixture of 8-(2,2′-dichloro-3′-(5-((3-(hydroxymethyl)azetidin-1-yl)methyl)-6-methoxypyrazin-2-yl)-[1,1′-biphenyl]-3-yl)-4-oxo-4H-pyrido[1,2-a]pyrimidine-3-carbaldehyde (160 mg, 0.27 mmol) and (S)-pyrrolidine-3-carboxylic acid (0.06 g, 0.53 mmol) in CH₂Cl₂ (6 mL) was added sodium acetate (0.04 g, 0.53 mmol), 4 Å molecular sieve (0.15 g) and sodium triacetoxyborohydride (0.17 g, 0.8 mmol) in one portion under N₂. The mixture was stirred at r.t. for 12 h. The mixture was filtered. The filter cake was washed with MeOH (20 mL). The filtrate was concentrated. The residue was purified by reverse phase HPLC to afford (S)-1-((8-(2,2′-dichloro-3′-(5-((3-(hydroxymethyl)azetidin-1-yl)methyl)-6-methoxypyrazin-2-yl)-[1,1′-biphenyl]-3-yl)-4-oxo-4H-pyrido[1,2-a]pyrimidin-3-yl)methyl)pyrrolidine-3-carboxylic acid (31 mg, 16% yield) as a light yellow solid. m/z found: 701 [M+H]⁺. ¹H NMR (400 MHz, Methanol-d₄) δ 9.18 (d, J=7.6 Hz, 1H), 8.54 (s, 1H), 8.43 (s, 1H), 7.84 (d, J=1.6 Hz, 1H), 7.72 (d, J=7.6 Hz, J=1.6 Hz, 1H), 7.70-7.53 (m, 5H), 7.48 (dd, J=7.6 Hz, J=1.6 Hz, 1H), 4.28 (s, 2H), 4.09-4.08 (m, 5H), 3.80 (t, J=8.4 Hz, 2H), 3.70 (d, J=6 Hz, 2H), 3.52 (t, J=8 Hz, 2H), 3.47-3.38 (m, 2H), 3.30-3.28 (m, 2H), 3.12-3.05 (m, 1H), 2.88-2.80 (m, 1H), 2.36-2.19 (m, 2H).

### Example 93: 1-((8-(2,2′-Dichloro-3′-(5-((3-(hydroxymethyl)azetidin-1-yl)methyl)-6-methoxypyrazin-2-yl)-[1,1′-biphenyl]-3-yl)-4-oxo-4H-pyrido[1,2-a]pyrimidin-3-yl)methyl)azetidine-3-carboxylic acid

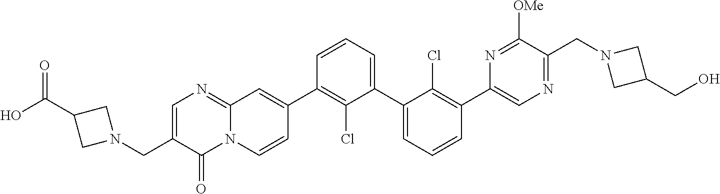 [SMILES: COc1nc(-c2cccc(-c3cccc(-c4ccn5c(=O)c(CN6CC(C(=O)O)C6)cnc5c4)c3Cl)c2Cl)cnc1CN1CC(CO)C1]

To a mixture of 8-(2,2′-dichloro-3′-(5-((3-(hydroxymethyl)azetidin-1-yl)methyl)-6-methoxypyrazin-2-yl)-[1,1′-biphenyl]-3-yl)-4-oxo-4H-pyrido[1,2-a]pyrimidine-3-carbaldehyde (220 mg, 0.37 mmol) and azetidine-3-carboxylic acid (74 mg, 0.73 mmol) in CH₂C2 (6 mL) was added sodium acetate (60 mg, 0.73 mmol), sodium triacetoxyborohydride (232 mg, 1.1 mmol) and 4 Å molecular sieve (150 mg) in one portion under N₂. The mixture was stirred at r.t. for 12 h and was filtered. The filter cake was washed with MeOH (20 mL). The filtrate was concentrated. The residue was purified by reverse phase HPLC to afford 1-((8-(2,2′-dichloro-3′-(5-((3-(hydroxymethyl)azetidin-1-yl)methyl)-6-methoxypyrazin-2-yl)-[1,1′-biphenyl]-3-yl)-4-oxo-4H-pyrido[1,2-a]pyrimidin-3-yl)methyl)azetidine-3-carboxylic acid (40 mg, 15% yield) as a white solid. m/z found: 687 [M+H]⁺. ¹H NMR (400 MHz, Methanol-d₄) δ 9.16 (d, J=7.2 Hz, 1H), 8.49 (s, 1H), 8.43 (s, 1H), 7.83 (d, J=1.6 Hz, 1H), 7.72 (d, J=8 Hz, J=1.6 Hz, 1H), 7.67-7.52 (m, 5H), 7.49 (dd, J=7.6 Hz, J=1.6 Hz, 1H), 4.21 (s, 2H), 4.15 (s, 2H), 4.12-4.01 (m, 7H), 3.87 (t, J=8.4 Hz, 2H), 3.71 (d, J=6 Hz, 2H), 3.59 (t, J=8 Hz, 2H), 3.42-3.36 (m, 2H), 2.90-2.83 (m, 1H).

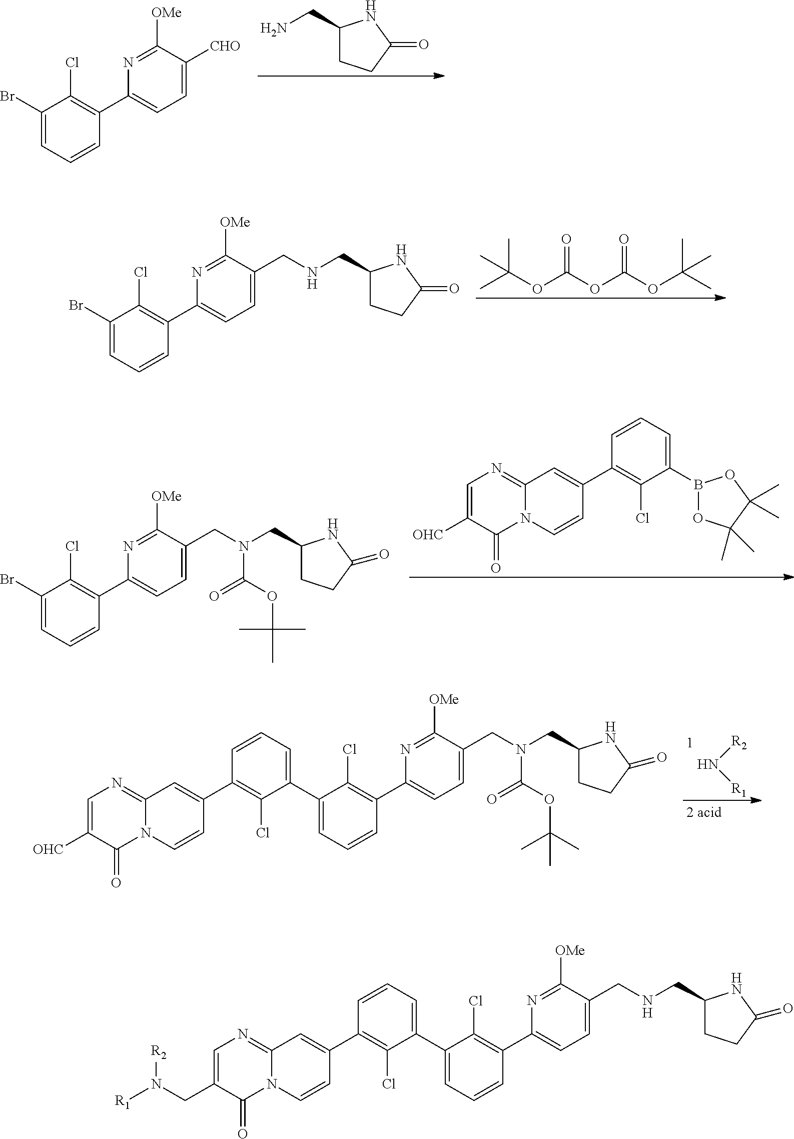

### Example 94: (S)-1-((8-(2,2′-Dichloro-3′-(6-methoxy-5-(((((S)-5-oxopyrrolidin-2-yl)methyl)amino)methyl)pyridin-2-yl)-[1,1′-biphenyl]-3-yl)-4-oxo-4H-pyrido[1,2-a]pyrimidin-3-yl)methyl)pyrrolidine-3-carboxylic acid

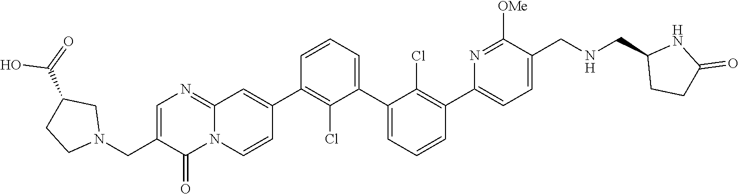 [SMILES: COc1nc(-c2cccc(-c3cccc(-c4ccn5c(=O)c(CN6CC[C@H](C(=O)O)C6)cnc5c4)c3Cl)c2Cl)ccc1CNC[C@@H]1CCC(=O)N1]

### (S)-5-((((6-(3-Bromo-2-chlorophenyl)-2-methoxypyridin-3-yl)methyl)amino) methyl) pyrrolidin-2-one

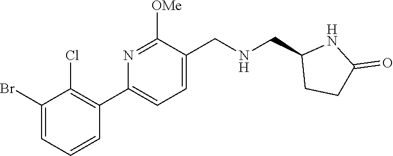 [SMILES: COc1nc(-c2cccc(Br)c2Cl)ccc1CNC[C@@H]1CCC(=O)N1]

To a solution of 6-(3-bromo-2-chlorophenyl)-2-methoxynicotinaldehyde (3 g, 9.19 mmol) and (S)-5-(aminomethyl)pyrrolidin-2-one HCl (2.08 g, 13.8 mmol) in DCM (50 mL) was added sodium acetate (1.51 g, 18.4 mmol). The mixture was stirred at r.t. for 0.5 h, then sodium triacetoxyborohydride (2.92 g, 13.8 mmol) was added. The reaction was stirred at r.t. for 1.5 hr, at which point (S)-5-((((6-(3-bromo-2-chlorophenyl)-2-methoxypyridin-3-yl)methyl) amino)methyl)pyrrolidin-2-one (3.9 g, crude, m/z: 424 [M+H]⁺ observed (LCMS: Method A)) in 50 mL of CH₂Cl₂ was obtained as a yellow liquid.

### (S)-tert-Butyl ((6-(3-bromo-2-chlorophenyl)-2-methoxypyridin-3-yl)methyl)((5-oxopyrrolidin-2-yl)methyl)carbamate

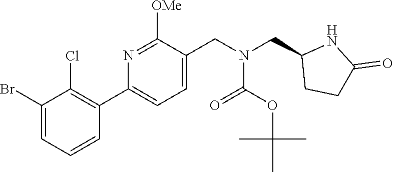 [SMILES: COc1nc(-c2cccc(Br)c2Cl)ccc1CN(C[C@@H]1CCC(=O)N1)C(=O)OC(C)(C)C]

To a solution of (S)-5-((((6-(3-bromo-2-chlorophenyl)-2-methoxypyridin-3-yl)methyl)amino)methyl)pyrrolidin-2-one (3.9 g, 9.18 mmol, 1 eq) in CH₂Cl₂ (70 mL) was added triethylamine (2.56 mL, 18.4 mmol) and di-tert-butyl dicarbonate (4.01 g, 18.4 mmol). The mixture was stirred at r.t. for 12 hr. H₂O (50 mL), then was extracted with CH₂Cl₂ (40 mL). The combined organic layers were dried over anhydrous sodium sulfate, filtered and concentrated. The residue was purified by normal phase SiO₂ chromatography (0-100% EtOAc/petroleum ether) to afford (S)-tert-butyl ((6-(3-bromo-2-chlorophenyl)-2-methoxypyridin-3-yl)methyl)((5-oxopyrrolidin-2-yl)methyl)carbamate (2 g, 42% yield for two steps) as a white solid. ¹H NMR (400 MHz, DMSO-d₆) δ 7.85 (d, J=6.4 Hz, 1H), 7.77 (s, 1H), 7.58 (dd, J=7.6 Hz, J=1.6 Hz, 1H), 7.39 (t, J=8 Hz, 1H), 7.24 (d, J=7.2 Hz, 1H), 4.47-4.34 (m, 2H), 3.91 (s, 3H), 3.78-3.76 (m, 1H), 3.31-3.26 (m, 2H), 2.18-2.06 (m, 3H), 1.74 (m, 1H), 1.40 (d, J=52.8 Hz, 9H).

### (S)-tert-Butyl ((6-(2,2′-dichloro-3′-(3-formyl-4-oxo-4H-pyrido[1,2-a]pyrimidin-8-yl)-[1,1′-biphenyl]-3-yl)-2-methoxypyridin-3-yl)methyl)((5-oxopyrrolidin-2-yl)methyl)carbamate

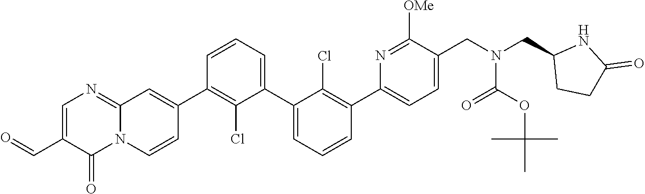 [SMILES: COc1nc(-c2cccc(-c3cccc(-c4ccn5c(=O)c(C=O)cnc5c4)c3Cl)c2Cl)ccc1CN(C[C@@H]1CCC(=O)N1)C(=O)OC(C)(C)C]

To a solution of (S)-tert-butyl ((6-(3-bromo-2-chlorophenyl)-2-methoxypyridin-3-yl)methyl)((5-oxopyrrolidin-2-yl)methyl)carbamate (650 mg, 1.24 mmol) and 8-(2-chloro-3-(4,4,5,5-tetramethyl-1,3,2-dioxaborolan-2-yl)phenyl)-4-oxo-4H-pyrido[1,2-a]pyrimidine-3-carbaldehyde (763 mg, 1.86 mmol) in dioxane (20 mL) and H₂O (2 mL), was added potassium phosphate (526 mg, 2.48 mmol) and [1,1-bis(di-tert-butylphosphino)ferrocene]palladium(II) Dichloride (80.7 mg, 0.12 mmol). The reaction was stirred at 110° C. for 0.5 hr. H₂O (15 mL). The mixture was extracted with EtOAc (2×20 mL). The combined organic layers were dried over anhydrous sodium sulfate, filtered and concentrated. The residue was purified by normal phase SiO₂ chromatography (5-10% MeOH/Ethyl acetate) to afford 700 mg of (S)-tert-butyl ((6-(2,2′-dichloro-3′-(3-formyl-4-oxo-4H-pyrido[1,2-a]pyrimidin-8-yl)-[1,1′-biphenyl]-3-yl)-2-methoxypyridin-3-yl)methyl)((5-oxopyrrolidin-2-yl)methyl)carbamate as a yellow solid. ¹H NMR (400 MHz, DMSO-d₆): δ10.20 (s, 1H), 9.29 (d, J=7.2 Hz, 1H), 8.80 (s, 1H), 8.05 (s, 1H), 7.76-7.31 (m, 10H), 4.47-4.35 (m, 2H), 3.94 (s, 3H), 3.78-3.76 (m, 1H), 3.31-3.27 (m, 2H), 2.18-2.06 (m, 3H), 1.75 (m, 1H), 1.44-1.31 (d, J=54 Hz, 9H).

### (S)-1-((8-(3′-(5-(((tert-Butoxycarbonyl)(((S)-5-oxopyrrolidin-2-yl)methyl)amino)methyl)-6-methoxypyridin-2-yl)-2,2′-dichloro-[1,1′-biphenyl]-3-yl)-4-oxo-4H-pyrido[1,2-a]pyrimidin-3-yl)methyl)pyrrolidine-3-carboxylic acid

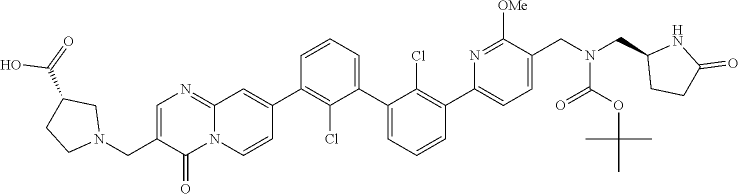 [SMILES: COc1nc(-c2cccc(-c3cccc(-c4ccn5c(=O)c(CN6CC[C@H](C(=O)O)C6)cnc5c4)c3Cl)c2Cl)ccc1CN(C[C@@H]1CCC(=O)N1)C(=O)OC(C)(C)C]

To a solution of (S)-tert-butyl ((6-(2,2′-dichloro-3′-(3-formyl-4-oxo-4H-pyrido[1,2-a]pyrimidin-8-yl)-[1,1′-biphenyl]-3-yl)-2-methoxypyridin-3-yl)methyl)((5-oxopyrrolidin-2-yl)methyl)carbamate (300 mg, 0.41 mmol) and (S)-pyrrolidine-3-carboxylic acid (142 mg, 1.24 mmol) in DCM (20 mL) was added 4 Å molecular sieve (300 mg) and potassium acetate (203 mg, 2.47 mmol). The mixture was stirred at r.t. for 1 hr. Sodium triacetoxyborohydride (262 mg, 1.24 mmol) was added to the reaction, which was then stirred at r.t. for 11 hr. The reaction mixture was filtered and concentrated, yielding (S)-1-((8-(3′-(5-(((tert-butoxycarbonyl)(((S)-5-oxopyrrolidin-2-yl)methyl)amino)methyl)-6-methoxypyridin-2-yl)-2,2′-dichloro-[1,1′-biphenyl]-3-yl)-4-oxo-4H-pyrido[1,2-a]pyrimidin-3-yl)methyl)pyrrolidine-3-carboxylic acid as a crude yellow oil (400 mg, crude, m/z: 827 [M+H]⁺ observed (LCMS: Method A).

### (S)-1-((8-(2,2′-Dichloro-3′-(6-methoxy-5-(((((S)-5-oxopyrrolidin-2-yl)methyl)amino) methyl)pyridin-2-yl)-[1,1′-biphenyl]-3-yl)-4-oxo-4H-pyrido[1,2-a]pyrimidin-3-yl)methyl) pyrrolidine-3-carboxylic acid

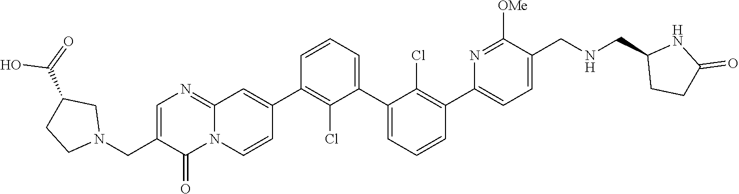 [SMILES: COc1nc(-c2cccc(-c3cccc(-c4ccn5c(=O)c(CN6CC[C@H](C(=O)O)C6)cnc5c4)c3Cl)c2Cl)ccc1CNC[C@@H]1CCC(=O)N1]

To a solution of (S)-1-((8-(3′-(5-(((tert-butoxycarbonyl)(((S)-5-oxopyrrolidin-2-yl)methyl)amino)methyl)-6-methoxypyridin-2-yl)-2,2′-dichloro-[1,1′-biphenyl]-3-yl)-4-oxo-4H-pyrido[1,2-a]pyrimidin-3-yl)methyl)pyrrolidine-3-carboxylic acid (380 mg, 0.46 mmol) in CH₂Cl₂ (15 mL) was added trifluoroacetic acid (3 mL, 40.5 mmol). The mixture was stirred at r.t. for 0.5 hr. The reaction mixture was concentrated. The residue was purified by reverse phase HPLC to afford (S)-1-((8-(2,2′-dichloro-3′-(6-methoxy-5-(((((S)-5-oxopyrrolidin-2-yl)methyl)amino)methyl)pyridin-2-yl)-[1,1′-biphenyl]-3-yl)-4-oxo-4H-pyrido[1,2-a]pyrimidin-3-yl)methyl)pyrrolidine-3-carboxylic acid (113 mg, 34% yield, m/z: 727 [M+H]⁺ observed (LCMS: Method B)) as a white solid. ¹H NMR (400 MHz, CD₃OD) δ9.20 (d, J=7.6 Hz, 1H), 8.56 (s, 1H), 7.87 (d, J=1.6 Hz, 1H), 7.76 (d, J=7.6 Hz, 1H), 7.69-7.62 (m, 4H), 7.55-7.52 (m, 2H), 7.41 (dd, J=7.2 Hz, J=1.6 Hz, 1H), 7.26 (d, J=7.6 Hz, 1H), 4.42 (s, 2H), 4.04 (s, 3H), 3.88-3.85 (m, 3H), 3.65-3.60 (m, 1H), 3.50-3.49 (m, 3 H), 3.16-3.08 (m, 1H), 2.79-2.72 (m, 2H), 2.42-2.26 (m, 5H), 1.88-1.79 (m, 1H).

### Example 95: 8-(2-Chloro-3′-(6-methoxy-5-(((((S)-5-oxopyrrolidin-2-yl)methyl)amino)methyl)pyridin-2-yl)-2′-(trifluoromethyl)-[1,1′-biphenyl]-3-yl)-3-(((((S)-5-oxopyrrolidin-2-yl)methyl)amino)methyl)-4H-pyrido[1,2-a]pyrimidin-4-one

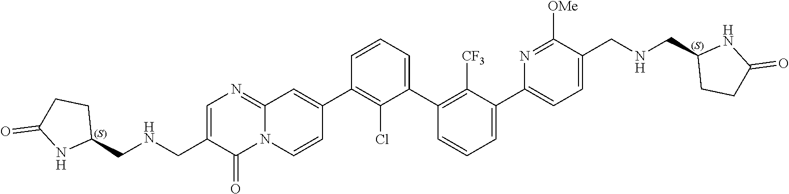

### 8-(3′-Bromo-2-chloro-2′-(trifluoromethyl)-[1,1′-biphenyl]-3-yl)-4-oxo-4H-pyrido[1,2-a]pyrimidine-3-carbaldehyde

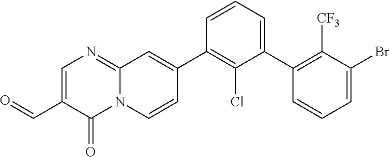 [SMILES: O=Cc1cnc2cc(-c3cccc(-c4cccc(Br)c4C(F)(F)F)c3Cl)ccn2c1=O]

To a solution of 8-(2-chloro-3-(4,4,5,5-tetramethyl-1,3,2-dioxaborolan-2-yl)phenyl)-4-oxo-4H-pyrido[1,2-a]pyrimidine-3-carbaldehyde (0.1 g, 0.24 mmol) and 1,3-dibromo-2-(trifluoromethyl)benzene (0.07 g, 0.24 mmol) in dioxane (10 mL) under nitrogen gas was added 1,1′-bis(diphenylphosphino)ferrocene-palladium(ii) dichloride (DCM adduct) (0.01 g, 0.02 mmol) and potassium carbonate (0.08 g, 0.61 mmol). The mixture was stirred at 90° C. for 3 hrs. The mixture was filtered through CELITE® and the filter cake was washed with EtOAc (3×40 mL). The filtrate was washed with saturated aqueous brine solution (2×30 mL), then dried over Na₂SO₄ and concentrated under reduced pressure. The residue was purified by normal phase flash SiO₂ chromatography using a linear gradient (0-40% ethyl acetate hexanes) to afford 8-(3′-bromo-2-chloro-2′-(trifluoromethyl)-[1,1′-biphenyl]-3-yl)-4-oxo-4H-pyrido[1,2-a]pyrimidine-3-carbaldehyde (0.036 g, 29% yield). MS: m/z found 506 [M+H]⁺.

### 8-(2-Chloro-3′-(5-formyl-6-methoxypyridin-2-yl)-2′-(trifluoromethyl)-[1,1′-biphenyl]-3-yl)-4-oxo-4H-pyrido[1,2-a]pyrimidine-3-carbaldehyde

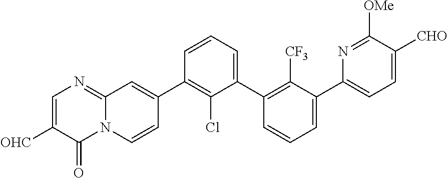 [SMILES: COc1nc(-c2cccc(-c3cccc(-c4ccn5c(=O)c(C=O)cnc5c4)c3Cl)c2C(F)(F)F)ccc1C=O]

To a solution of 8-(3′-bromo-2-chloro-2′-(trifluoromethyl)-[1,1′-biphenyl]-3-yl)-4-oxo-4H-pyrido[1,2-a]pyrimidine-3-carbaldehyde (0.03 g, 0.05 mmol) and (5-formyl-6-methoxypyridin-2-yl)boronic acid (0.02 g, 0.08 mmol) in dioxane (10 mL) under nitrogen gas was added 1,1′-bis(diphenylphosphino)ferrocene-palladium(ii) dichloride (DCM adduct) (0.004 g, 0.005 mmol) and potassium carbonate (0.02 g, 0.12 mmol). The mixture was stirred at 90° C. for 3 hrs. The mixture was filtered through CELITE® and the filter cake was washed with EtOAc (3×40 mL). The filtrate was washed with saturated aqueous brine solution (2×30 mL), then dried over Na₂SO₄ and concentrated under reduced pressure. The residue was purified by normal phase flash SiO₂ chromatography using a linear gradient (0-3% MeOH/DCM) to afford 8-(2-chloro-3′-(5-formyl-6-methoxypyridin-2-yl)-2′-(trifluoromethyl)-[1,1′-biphenyl]-3-yl)-4-oxo-4H-pyrido[1,2-a]pyrimidine-3-carbaldehyde (0.02 g, 71% yield). MS: m/z found 564 [M+H]⁺.

### 8-(2-Chloro-3′-(6-methoxy-5-(((((S)-5-oxopyrrolidin-2-yl)methyl)amino)methyl)pyridin-2-yl)-2′-(trifluoromethyl)-[1,1′-biphenyl]-3-yl)-3-(((((S)-5-oxopyrrolidin-2-yl)methyl)amino)methyl)-4H-pyrido[1,2-a]pyrimidin-4-one

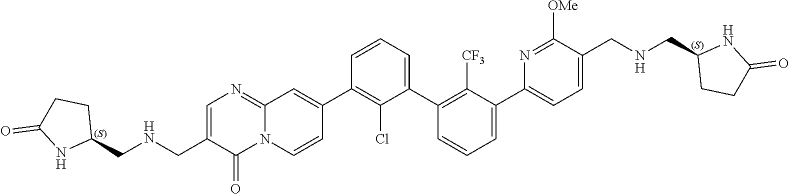

To a mixture of 8-(2-chloro-3′-(5-formyl-6-methoxypyridin-2-yl)-2′-(trifluoromethyl)-[1,1′-biphenyl]-3-yl)-4-oxo-4H-pyrido[1,2-a]pyrimidine-3-carbaldehyde (0.02 g, 0.04 mmol) and (S)-5-(aminomethyl)pyrrolidin-2-one (0.01 g, 0.12 mmol) in 1:1 THF/MeOH (6 mL) was added acetic acid (0.01 g, 0.11 mmol) and 4 Å molecular sieve (1 g). The mixture was stirred at r.t. for 3 hrs. Sodium cyanoborohydride (0.01 g, 0.12 mmol) was added and the mixture was stirred at r.t. for 1 hr. The reaction was concentrated, and residue purified by Gilson. ACN was evaporated off, then the aqueous layer was brought to pH 9 with saturated NaHCO₃, then extracted with 3:1 CHCl₃/IPA (3×40 mL). Combined organic was washed with brine, then dried over Na₂SO₄. Characterization at this point showed an impurity, so material was purified on ISCO using a liner gradient of 0-10% MeOH (containing 1% 7M NH₃ in MeOH) to give 5 mg (19%) of the product as free base affording 8-(2-chloro-3′-(6-methoxy-5-(((((S)-5-oxopyrrolidin-2-yl)methyl)amino)methyl)pyridin-2-yl)-2′-(trifluoromethyl)-[1,1′-biphenyl]-3-yl)-3-(((((S)-5-oxopyrrolidin-2-yl)methyl)amino)methyl)-4H-pyrido[1,2-a]pyrimidin-4-one as a white solid. MS: m/z found 760 [M+H]⁺. ¹H NMR (400 MHz, Chloroform-d) δ 9.08 (d, J=7.4 Hz, 1H), 8.37 (s, 1H), 7.75 (s, 1H), 7.64 (t, J=7.5 Hz, 2H), 7.54 (d, J=7.7 Hz, 1H), 7.50-7.42 (m, 3H), 7.36 (d, J=7.6 Hz, 2H), 7.04 (d, J=7.4 Hz, 1H), 6.35 (s, 1H), 6.23 (s, 1H), 4.00 (t, J=0.8 Hz, 3H), 3.94 (d, 2H), 3.90-3.85 (m, 2H), 2.88 (t, J=14.6 Hz, 2H), 2.71-2.57 (m, 2H), 2.41-2.31 (m, 4H), 2.32-2.19 (m, 2H), 1.84-1.70 (m, 1H), 1.28-1.23 (m, 5H).

### Example 96: 8-(3′-(6-Methoxy-5-(((((S)-5-oxopyrrolidin-2-yl)methyl)amino)methyl)pyridin-2-yl)-2-methyl-2′-(trifluoromethyl)-[1,1′-biphenyl]-3-yl)-3-(((((S)-5-oxopyrrolidin-2-yl)methyl)amino)methyl)-4H-pyrido[1,2-a]pyrimidin-4-one

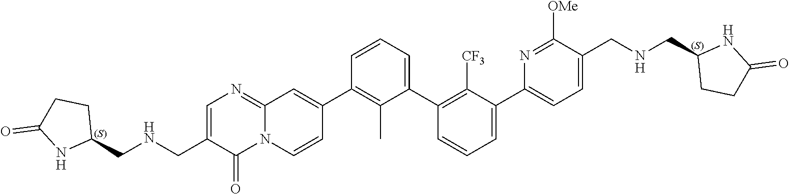

### 8-(3′-Bromo-2-methyl-2′-(trifluoromethyl)-[1,1′-biphenyl]-3-yl)-4-oxo-4H-pyrido[1,2-a]pyrimidine-3-carbaldehyde

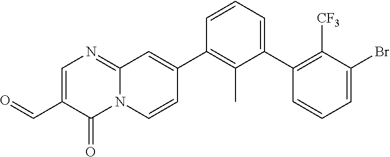 [SMILES: Cc1c(-c2ccn3c(=O)c(C=O)cnc3c2)cccc1-c1cccc(Br)c1C(F)(F)F]

To a solution of 8-(2-methyl-3-(4,4,5,5-tetramethyl-1,3,2-dioxaborolan-2-yl)phenyl)-4-oxo-4H-pyrido[1,2-a]pyrimidine-3-carbaldehyde (0.15 g, 0.38 mmol) and 1,3-dibromo-2-(trifluoromethyl)benzene (0.18 g, 0.58 mmol) in dioxane (10 mL) under nitrogen gas was added 1,1′-bis(diphenylphosphino)ferrocene-palladium(ii) dichloride (DCM adduct) (0.02 g, 0.02 mmol) and potassium carbonate (0.13 g, 0.96 mmol). The mixture was stirred at 90° C. for 3 hrs. The mixture was filtered through CELITE® and the filter cake was washed with EtOAc (3×40 mL). The filtrate was washed with saturated aqueous brine solution (2×30 mL), then dried over Na₂SO₄ and concentrated under reduced pressure. The residue was purified by normal phase flash SiO₂ chromatography using a linear gradient (0-40% ethyl acetate hexanes) to afford 8-(3′-bromo-2-methyl-2′-(trifluoromethyl)-[1,1′-biphenyl]-3-yl)-4-oxo-4H-pyrido[1,2-a]pyrimidine-3-carbaldehyde (0.04 g, 21% yield). MS: m/z found 487 [M+H]⁺.

### 8-(3′-(5-Formyl-6-methoxypyridin-2-yl)-2-methyl-2′-(trifluoromethyl)-[1,1′-biphenyl]-3-yl)-4-oxo-4H-pyrido[1,2-a]pyrimidine-3-carbaldehyde

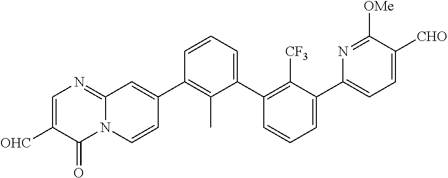 [SMILES: COc1nc(-c2cccc(-c3cccc(-c4ccn5c(=O)c(C=O)cnc5c4)c3C)c2C(F)(F)F)ccc1C=O]

To a solution of 8-(3′-bromo-2-methyl-2′-(trifluoromethyl)-[1,1′-biphenyl]-3-yl)-4-oxo-4H-pyrido[1,2-a]pyrimidine-3-carbaldehyde (0.04 g, 0.08 mmol) and (5-formyl-6-methoxypyridin-2-yl)boronic acid (0.02 g, 0.12 mmol) in dioxane (10 mL) under nitrogen gas was added 1,1′-bis(diphenylphosphino)ferrocene-palladium(ii) dichloride (DCM adduct) (0.01 g, 0.01 mmol) and potassium carbonate (0.03 g, 0.21 mmol). The mixture was stirred at 90° C. for 3 hrs. The mixture was filtered through CELITE® and the filter cake was washed with EtOAc (3×40 mL). The filtrate was washed with saturated aqueous brine solution (2×30 mL), then dried over Na₂SO₄ and concentrated under reduced pressure. The residue was purified by normal phase flash SiO₂ chromatography using a linear gradient (0-3% MeOH/DCM) to afford 8-(3′-(5-formyl-6-methoxypyridin-2-yl)-2-methyl-2′-(trifluoromethyl)-[1,1′-biphenyl]-3-yl)-4-oxo-4H-pyrido[1,2-a]pyrimidine-3-carbaldehyde (0.17 g, 38% yield). MS: m/z found 544 [M+H]⁺.

### 8-(3′-(6-Methoxy-5-(((((S)-5-oxopyrrolidin-2-yl)methyl)amino)methyl)pyridin-2-yl)-2-methyl-2′-(trifluoromethyl)-[1,1′-biphenyl]-3-yl)-3-(((((S)-5-oxopyrrolidin-2-yl)methyl)amino)methyl)-4H-pyrido[1,2-a]pyrimidin-4-one

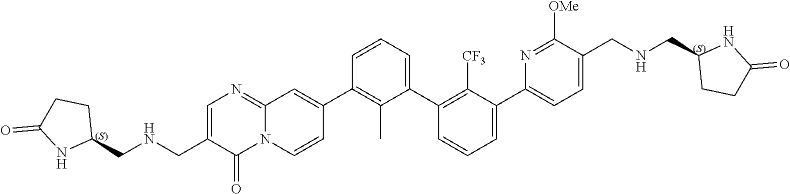

To a mixture of 8-(3′-(5-formyl-6-methoxypyridin-2-yl)-2-methyl-2′-(trifluoromethyl)-[1,1′-biphenyl]-3-yl)-4-oxo-4H-pyrido[1,2-a]pyrimidine-3-carbaldehyde (0.02 g, 0.04 mmol) and (S)-5-(aminomethyl)pyrrolidin-2-one (0.01 g, 0.13 mmol) in 1:1 THF/MeOH (6 mL) was added acetic acid (0.01 g, 0.11 mmol) and 4 Å molecular sieve (1 g). The mixture was stirred at r.t. for 3 hrs. Sodium cyanoborohydride (0.01 g, 0.13 mmol) was added and the mixture was stirred at r.t. for 1 hr. The reaction was concentrated, and residue purified by Gilson. ACN was evaporated off, then the aqueous layer was brought to pH=9 with saturated NaHCO₃, extracted with 3:1 CHCl₃/IPA (3×40 mL). Combined organic was washed with brine, then dried over Na₂SO₄. Characterization at this point showed an impurity, so material was purified on ISCO using a liner gradient of 0-10% MeOH (containing 1% 7M NH₃ in MeOH) to give 5 mg (19%) of the product as free base affording 8-(3′-(6-methoxy-5-(((((S)-5-oxopyrrolidin-2-yl)methyl)amino)methyl)pyridin-2-yl)-2-methyl-2′-(trifluoromethyl)-[1,1′-biphenyl]-3-yl)-3-(((((S)-5-oxopyrrolidin-2-yl)methyl)amino)methyl)-4H-pyrido[1,2-a]pyrimidin-4-one as a white solid. MS: m/z found 740 [M+H]⁺. ¹H NMR (400 MHz, Chloroform-d) δ 9.09 (d, 1H), 8.34 (s, 1H), 7.68-7.57 (m, 3H), 7.50 (d, J=7.6 Hz, 1H), 7.41-7.20 (m, 5H), 7.02 (d, 1H), 6.24 (s, 1H), 6.09 (s, 1H), 3.99 (s, 3H), 3.89 (d, 2H), 3.87-3.73 (m, 5H), 2.82 (t, J=12.5, 4.1 Hz, 2H), 2.65-2.53 (m, 2H), 2.41-2.18 (m, 6H), 2.09 (s, 3H), 1.84-1.69 (m, 1H), 1.79-1.74 (m, 2H), 1.25 (s, 2H).

### Example 97: 8-(2′-Chloro-5′-fluoro-3′-(6-methoxy-5-(((((S)-5-oxopyrrolidin-2-yl)methyl)amino)methyl)pyridin-2-yl)-2-methyl-[1,1′-biphenyl]-3-yl)-3-(((((S)-5-oxopyrrolidin-2-yl)methyl)amino)methyl)-4H-pyrido[1,2-a]pyrimidin-4-one

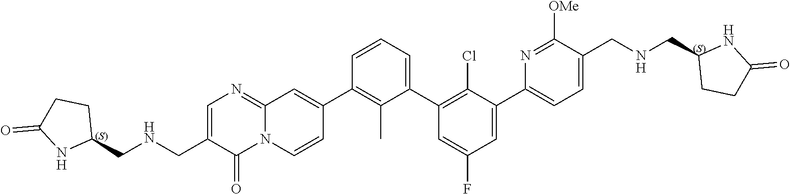

### 8-(3′-Bromo-2′-chloro-5′-fluoro-2-methyl-[1,1′-biphenyl]-3-yl)-4-oxo-4H-pyrido[1,2-a]pyrimidine-3-carbaldehyde

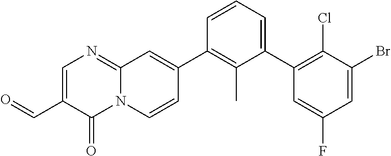 [SMILES: Cc1c(-c2ccn3c(=O)c(C=O)cnc3c2)cccc1-c1cc(F)cc(Br)c1Cl]

To a solution of 8-(2-methyl-3-(4,4,5,5-tetramethyl-1,3,2-dioxaborolan-2-yl)phenyl)-4-oxo-4H-pyrido[1,2-a]pyrimidine-3-carbaldehyde (0.15 g, 0.38 mmol) and 1,3-dibromo-2-chloro-5-fluorobenzene (0.17 g, 0.58 mmol) in dioxane (10 mL) under nitrogen gas was added 1,1′-bis(diphenylphosphino)ferrocene-palladium(II) dichloride (DCM adduct) (0.02 g, 0.02 mmol) and potassium carbonate (0.13 g, 0.96 mmol). The mixture was stirred at 90° C. for 3 hrs. The mixture was filtered through CELITE® and the filter cake was washed with EtOAc (3×40 mL). The filtrate was washed with saturated aqueous brine solution (2×30 mL), then dried over Na₂SO₄ and concentrated under reduced pressure. The residue was purified by normal phase flash SiO₂ chromatography using a linear gradient (0-40% ethyl acetate hexanes) to afford 8-(3′-bromo-2′-chloro-5′-fluoro-2-methyl-[1,1′-biphenyl]-3-yl)-4-oxo-4H-pyrido[1,2-a]pyrimidine-3-carbaldehyde (0.06 g, 33% yield). MS: m/z found 470 [M+H]⁺.

### 8-(2′-Chloro-5′-fluoro-3′-(5-formyl-6-methoxypyridin-2-yl)-2-methyl-[1,1′-biphenyl]-3-yl)-4-oxo-4H-pyrido[1,2-a]pyrimidine-3-carbaldehyde

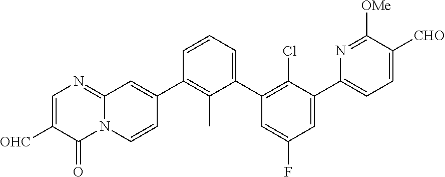 [SMILES: COc1nc(-c2cc(F)cc(-c3cccc(-c4ccn5c(=O)c(C=O)cnc5c4)c3C)c2Cl)ccc1C=O]

To a solution of 8-(3′-bromo-2′-chloro-5′-fluoro-2-methyl-[1,1′-biphenyl]-3-yl)-4-oxo-4H-pyrido[1,2-a]pyrimidine-3-carbaldehyde (0.06 g, 0.13 mmol) and (5-formyl-6-methoxypyridin-2-yl)boronic acid (0.03 g, 0.17 mmol) in dioxane (10 mL) under nitrogen gas was added 1,1′-bis(diphenylphosphino)ferrocene-palladium(II) dichloride (DCM adduct) (0.01 g, 0.01 mmol) and potassium carbonate (0.04 g, 0.32 mmol). The mixture was stirred at 90° C. for 3 hrs. The mixture was filtered through CELITE® and the filter cake was washed with EtOAc (3×40 mL). The filtrate was washed with saturated aqueous brine solution (2×30 mL), then dried over Na₂SO₄ and concentrated under reduced pressure. The residue was purified by normal phase flash SiO₂ chromatography using a linear gradient (0-3% MeOH/DCM) to afford 8-(2′-chloro-5′-fluoro-3′-(5-formyl-6-methoxypyridin-2-yl)-2-methyl-[1,1′-biphenyl]-3-yl)-4-oxo-4H-pyrido[1,2-a]pyrimidine-3-carbaldehyde (0.28 g, 42% yield). MS: m/z found 528 [M+H]⁺.

### 8-(2′-Chloro-5′-fluoro-3′-(6-methoxy-5-(((((S)-5-oxopyrrolidin-2-yl)methyl)amino)methyl)pyridin-2-yl)-2-methyl-[1,1′-biphenyl]-3-yl)-3-(((((S)-5-oxopyrrolidin-2-yl)methyl)amino)methyl)-4H-pyrido[1,2-a]pyrimidin-4-one

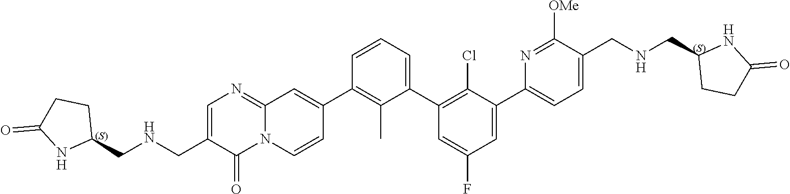

To a mixture of 8-(2′-chloro-5′-fluoro-3′-(5-formyl-6-methoxypyridin-2-yl)-2-methyl-[1,1′-biphenyl]-3-yl)-4-oxo-4H-pyrido[1,2-a]pyrimidine-3-carbaldehyde (0.03 g, 0.06 mmol) and (S)-5-(aminomethyl)pyrrolidin-2-one (0.02 g, 0.20 mmol) in 1:1 THF/MeOH (6 mL) was added acetic acid (0.01 g, 0.17 mmol) and 4 Å molecular sieve (1 g). The mixture was stirred at r.t. for 3 hrs. Sodium cyanoborohydride (0.01 g, 0.20 mmol) was added and the mixture was stirred at r.t. for 1 hr. The reaction was concentrated, and residue purified by Gilson. ACN was evaporated off, then the aqueous layer was brought to pH=9 with saturated NaHCO₃, extracted with 3:1 CHCl₃/IPA (3×40 mL). Combined organic was washed with brine, then dried over Na₂SO₄. Characterization at this point showed an impurity, so material was purified on ISCO using a liner gradient of 0-10% MeOH (containing 1% 7M NH₃ in MeOH) to give 9 mg (22%) of the product as free base affording 8-(2′-chloro-5′-fluoro-3′-(6-methoxy-5-(((((S)-5-oxopyrrolidin-2-yl)methyl)amino)methyl)pyridin-2-yl)-2-methyl-[1,1′-biphenyl]-3-yl)-3-(((((S)-5-oxopyrrolidin-2-yl)methyl)amino)methyl)-4H-pyrido[1,2-a]pyrimidin-4-one as a white solid. MS: m/z found 724 [M+H]⁺. ¹H NMR (400 MHz, Chloroform-d) δ 9.09 (d, J=7.3 Hz, 1H), 8.33 (s, 1H), 7.68-7.58 (m, 2H), 7.46-7.28 (m, 4H), 7.32-7.20 (m, 1H), 7.05 (d, J=8.0, 3.1 Hz, 1H), 6.17 (s, 1H), 6.00 (s, 1H), 4.03 (s, 3H), 3.96-3.82 (m, 2H), 3.85-3.77 (m, 4H), 2.83 (t, J=11.3, 4.1 Hz, 2H), 2.65-2.53 (m, 2H), 2.41-2.31 (m, 3H), 2.32-2.20 (m, 1H), 2.14 (s, 3H), 1.84-1.71 (m, 2H), 1.35-1.20 (m, 4H).

### Example 98: (S)-1-((8-(2,2′-Dichloro-3′-(6-methoxy-5-((((5-oxopyrrolidin-2-yl)methyl)amino)methyl)pyridin-2-yl)-[1,1′-biphenyl]-3-yl)-4-oxo-4H-pyrido[1,2-a]pyrimidin-3-yl)methyl)azetidine-3-carboxylic acid

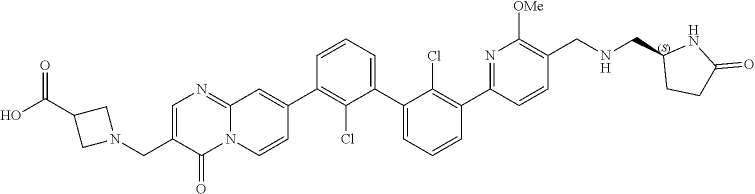

### (S)-1-((8-(3′-(5-(((tert-Butoxycarbonyl)((5-oxopyrrolidin-2-yl)methyl)amino)methyl)-6-methoxypyridin-2-yl)-2,2′-dichloro-[1,1′-biphenyl]-3-yl)-4-oxo-4H-pyrido[1,2-a]pyrimidin-3-yl)methyl)azetidine-3-carboxylic acid

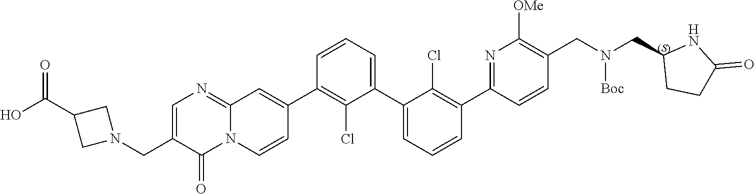

To a solution of (S)-tert-butyl ((6-(2,2′-dichloro-3′-(3-formyl-4-oxo-4H-pyrido[1,2-a]pyrimidin-8-yl)-[1,1′-biphenyl]-3-yl)-2-methoxypyridin-3-yl)methyl)((5-oxopyrrolidin-2-yl)methyl)carbamate (300 mg, 0.41 mmol) and azetidine-3-carboxylic acid (125 mg, 1.24 mmol) in CH₂Cl₂ (20 mL) was added 4 Å molecular sieve (300 mg) and sodium acetate (203 mg, 2.47 mmol). The mixture was stirred at r.t. for 0.5 hr. Sodium triacetoxyborohydride (262 mg, 1.24 mmol) was added to the mixture. The mixture was stirred at r.t. for 11.5 hr. The reaction mixture was filtered and filtrate was concentrated to give (S)-1-((8-(3′-(5-(((tert-butoxycarbonyl)((5-oxopyrrolidin-2-yl)methyl)amino)methyl)-6-methoxypyridin-2-yl)-2,2′-dichloro-[1,1′-biphenyl]-3-yl)-4-oxo-4H-pyrido[1,2-a]pyrimidin-3-yl)methyl)azetidine-3-carboxylic acid (300 mg, crude) as a yellow oil. m/z found: 813 [M+H]⁺.

### (S)-1-((8-(2,2′-Dichloro-3′-(6-methoxy-5-((((5-oxopyrrolidin-2-yl)methyl)amino)methyl)pyridin-2-yl)-[1,1′-biphenyl]-3-yl)-4-oxo-4H-pyrido[1,2-a]pyrimidin-3-yl)methyl)azetidine-3-carboxylic acid

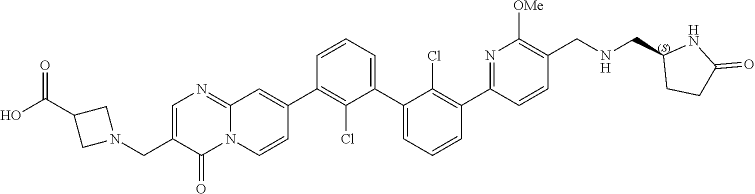

To a solution of (S)-1-((8-(3′-(5-(((tert-butoxycarbonyl)((5-oxopyrrolidin-2-yl)methyl)amino)methyl)-6-methoxypyridin-2-yl)-2,2′-dichloro-[1,1′-biphenyl]-3-yl)-4-oxo-4H-pyrido[1,2-a]pyrimidin-3-yl)methyl)azetidine-3-carboxylic acid (300 mg, 0.37 mmol) in CH₂Cl₂ (2 mL) was added trifluoroacetic acid (1.80 mL, 24.3 mmol). The mixture was stirred at r.t. for 0.5 hr, then concentrated. The residue was purified by reverse phase HPLC to afford (S)-1-((8-(2,2′-dichloro-3′-(6-methoxy-5-((((5-oxopyrrolidin-2-yl)methyl)amino)methyl)pyridin-2-yl)-[1,1′-biphenyl]-3-yl)-4-oxo-4H-pyrido[1,2-a]pyrimidin-3-yl)methyl)azetidine-3-carboxylic acid as formic acid salt (26.3 mg) as a white solid. m/z found: 713 [M+H]⁺. ¹H NMR (400 MHz, Methanol-d₄) δ 9.19 (d, J=7.6 Hz, 1H), 8.54 (s, 1H), 7.86 (d, J=1.6 Hz, 1H), 7.82 (d, J=7.6 Hz, 1H), 7.69-7.63 (m, 4H), 7.56-7.52 (m, 2H), 7.44 (dd, J=7.6 Hz, J=1.6 Hz, 1H), 7.31 (d, J=7.6 Hz, 1H), 4.44 (s, 2H), 4.31-4.28 (m, 4H), 4.07-4.05 (m, 5H), 3.97-3.93 (m, 1H), 3.52-3.42 (m, 1H), 2.96-2.94 (m, 2H), 2.38-2.35 (m, 3H), 1.92-1.83 (m, 1H).

### Example 99: (S)-5-((((6-(2,2′-Dichloro-3′-(6-(((((S)-5-oxopyrrolidin-2-yl)methyl)amino)methyl)imidazo[1,2-a]pyridin-2-yl)-[1,1′-biphenyl]-3-yl)-2-methoxypyridin-3-yl)methyl)amino)methyl)pyrrolidin-2-one

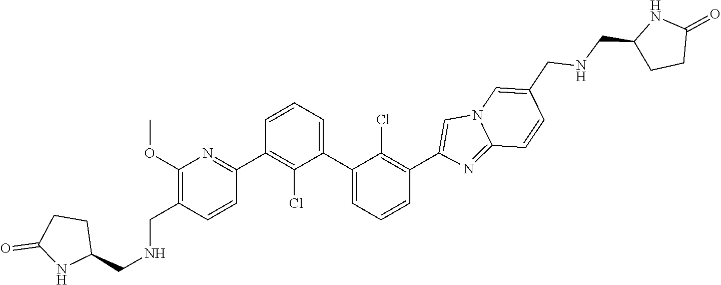 [SMILES: COc1nc(-c2cccc(-c3cccc(-c4cn5cc(CNC[C@@H]6CCC(=O)N6)ccc5n4)c3Cl)c2Cl)ccc1CNC[C@@H]1CCC(=O)N1]

### 2-(2,2′-Dichloro-3′-(5-formyl-6-methoxypyridin-2-yl)-[1,1′-biphenyl]-3-yl)-imidazo[1,2-a]-pyridine-6-carbaldehyde

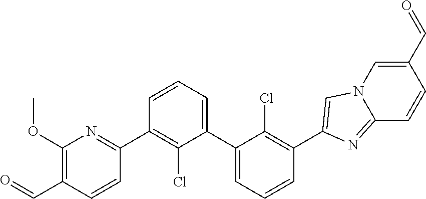 [SMILES: COc1nc(-c2cccc(-c3cccc(-c4cn5cc(C=O)ccc5n4)c3Cl)c2Cl)ccc1C=O]

1,1′-Bis(diphenylphosphino)-ferrocene-palladium(ii) dichloride dichloromethane complex (28 mg, 0.03 mmol), potassium carbonate (93 mg, 0.67 mmol), 2-[2-chloro-3-[2-chloro-3-(4,4,5,5-tetramethyl-1,3,2-dioxaborolan-2-yl)phenyl]phenyl]-4,4,5,5-tetramethyl-1,3,2-dioxaborolane (80 mg, 0.17 mmol), 2-bromoimidazo[1,2-a]pyridine-6-carbaldehyde (37.90 mg, 0.17 mmol) and 6-chloro-2-methoxy-pyridine-3-carbaldehyde (35 mg, 0.20 mmol) were suspended in 3 mL of 9:1 dioxane/water solvent mix. The solution was purged with nitrogen gas for 5 minutes, then heated at 95° C. for 1 hour. The reaction was cooled to rt, filtered through CELITE®, and the filtrate was concentrated. Crude sample was purified by silica gel chromatography, eluting with 0-50% EtOAc gradient in hexane to afford 2-[2-chloro-3-[2-chloro-3-(5-formyl-6-methoxy-2-pyridyl)phenyl]phenyl]imidazo[1,2-a]pyridine-6-carbaldehyde (45 mg, 53.2% yield) as a yellow solid. MS: m/z found 502.4, 504.4 [M+H]⁺.

### (S)-5-((((6-(2,2′-Dichloro-3′-(6-(((((S)-5-oxopyrrolidin-2-yl)methyl)amino)methyl)imidazo[1,2-a]pyridin-2-yl)-[1,1′-biphenyl]-3-yl)-2-methoxypyridin-3-yl)methyl)amino)methyl)pyrrolidin-2-one

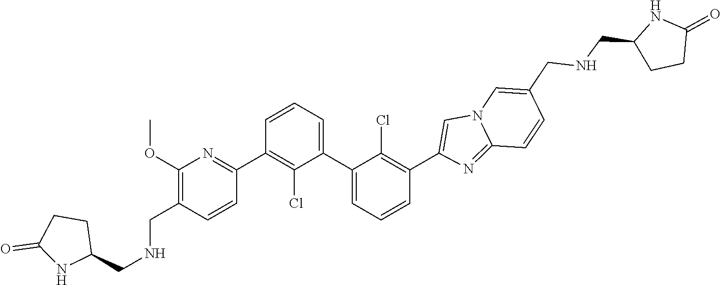 [SMILES: COc1nc(-c2cccc(-c3cccc(-c4cn5cc(CNC[C@@H]6CCC(=O)N6)ccc5n4)c3Cl)c2Cl)ccc1CNC[C@@H]1CCC(=O)N1]

2-[2-Chloro-3-[2-chloro-3-(5-formyl-6-methoxy-2-pyridyl)phenyl]phenyl]imidazo[1,2-a]pyridine-6-carbaldehyde (22 mg, 0.04 mmol), (5S)-5-(aminomethyl)pyrrolidin-2-one (15 mg, 0.13 mmol) and acetic acid (7 mg, 0.11 mmol) were dissolved in 1 mL of 1:1 MeOH/THF solvent mix, and the reaction was stirred at 25° C. for 3 hours. NaBH₃CN (8 mg, 0.13 mmol) was then added portion-wise, and the resulting mixture was stirred at 25° C. for 1 hour. The reaction was concentrated and the crude sample was purified by reverse phase HPLC to afford (5S)-5-[[[2-[2-chloro-3-[2-chloro-3-[6-methoxy-5-[[[(2S)-5-oxopyrrolidin-2-yl]methylamino]methyl]-2-pyridyl]phenyl]phenyl]imidazo[1,2-a]pyridin-6-yl]methylamino]methyl]pyrrolidin-2-one (10.1 mg, 33.0% yield) as a white foam. MS: m/z found 698.3, 700.3 [M+H]⁺. ¹H NMR (400 MHz, Chloroform-d) δ 8.30 (dd, J=7.8, 1.7 Hz, 1H), 8.25 (s, 1H), 8.12 (s, 1H), 7.67 (dd, J=7.7, 1.8 Hz, 1H), 7.59 (dd, J=11.9, 8.3 Hz, 2H), 7.45 (dt, J=10.4, 7.6 Hz, 2H), 7.33 (dd, J=7.6, 1.8 Hz, 1H), 7.30-7.26 (m, 2H), 7.17 (dd, J=9.3, 1.6 Hz, 1H), 6.35 (s, 1H), 6.18 (s, 1H), 4.03 (s, 3H), 3.87-3.71 (m, 6H), 2.80 (ddd, J=12.0, 7.8, 4.2 Hz, 2H), 2.57 (q, J=10.3 Hz, 2H), 2.36 (dd, J=9.2, 6.9 Hz, 4H), 2.25 (qd, J=16.1, 15.2, 6.9 Hz, 2H), 1.84-1.70 (m, 2H).

### Example 100: 2-(((2-(2,2′-Dichloro-3′-(5-(((2-hydroxyethyl)amino)methyl)-6-methoxypyridin-2-yl)-[1,1′-biphenyl]-3-yl)imidazo[1,2-a]pyridin-6-yl)methyl)amino)ethan-1-ol

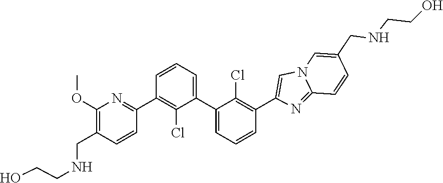 [SMILES: COc1nc(-c2cccc(-c3cccc(-c4cn5cc(CNCCO)ccc5n4)c3Cl)c2Cl)ccc1CNCCO]

2-[2-Chloro-3-[2-chloro-3-(5-formyl-6-methoxy-2-pyridyl)phenyl]phenyl]imidazo[1,2-a]pyridine-6-carbaldehyde (22 mg, 0.04 mmol), 2-aminoethanol (8 mg, 0.13 mmol) and acetic acid (7 mg, 0.11 mmol were dissolved in 1 ml of 1:1 MeOH/THF solvent mix, and the reaction was stirred at 25° C. for 3 hours. NaBH₃CN (8 mg, 0.13 mmol) was then added portion-wise, and the resulting mixture was stirred at 25° C. for 1 hour. The reaction was concentrated and the crude sample was purified by reverse phase HPLC to afford 2-[[2-[2-chloro-3-[2-chloro-3-[5-[(2-hydroxyethylamino)methyl]-6-methoxy-2-pyridyl]phenyl]phenyl]imidazo[1,2-a]pyridin-6-yl]methylamino]ethanol (6.2 mg, 23.9% yield) as a white foam. MS obsd. [(M+H)]: 592.3, 594.3. M+Na also observed. MS: m/z found 592.3, 594.3 [M+H]⁺. ¹H NMR (400 MHz, Chloroform-d) δ 8.33-8.27 (m, 1H), 8.25 (s, 1H), 8.12 (s, 1H), 7.70-7.64 (m, 1H), 7.64-7.56 (m, 2H), 7.49-7.40 (m, 2H), 7.33 (dd, J=7.4, 2.1 Hz, 1H), 7.29 (td, J=4.7, 2.2 Hz, 2H), 7.19 (d, J=9.1 Hz, 1H), 4.07-3.97 (m, 3H), 3.84 (d, J=6.1 Hz, 4H), 3.71 (dd, J=6.3, 4.0 Hz, 4H), 2.89-2.78 (m, 4H).

### Example 101: Methyl ((8-(2,2′-dichloro-3′-(6-methoxy-5-(((((S)-5-oxopyrrolidin-2-yl)methyl)amino)methyl)pyridin-2-yl)-[1,1′-biphenyl]-3-yl)-4-oxo-4H-pyrido[1,2-a]pyrimidin-3-yl)methyl)-L-serinate

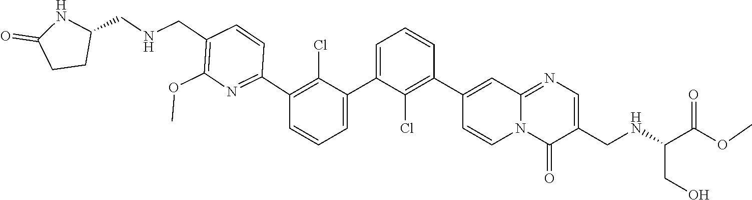 [SMILES: COC(=O)[C@H](CO)NCc1cnc2cc(-c3cccc(-c4cccc(-c5ccc(CNC[C@@H]6CCC(=O)N6)c(OC)n5)c4Cl)c3Cl)ccn2c1=O]

Methyl (2S)-2-amino-3-hydroxy-propanoate;hydrochloride (19 mg, 0.12 mmol), tert-butyl N-[[6-[2-chloro-3-[2-chloro-3-(3-formyl-4-oxo-pyrido[1,2-a]pyrimidin-8-yl)phenyl]phenyl]-2-methoxy-3-pyridyl]methyl]-N-[[(2S)-5-oxopyrrolidin-2-yl]methyl]carbamate (30 mg, 0.04 mmol) and DIEA (36 μL, 27 mg, 0.21 mmol) were dissolved in 1 ml of 1:1 MeOH/THF solvent mix, and the reaction was stirred at 25° C. for 3 hours. NaBH₃CN (7.76 mg, 0.12 mmol), along with acetic acid, (8 mg, 0.13 mmol) were then added, and the resulting mixture was stirred at 25° C. for 1 hour. The reaction mixture was diluted with water (2 mL) and extracted with DCM (3×2 mL). The combined organics were further washed with brine, dried over sodium sulfate, then filtered and concentrated. Crude mixture was purified by silica gel chromatography, eluting with 0-12% MeOH gradient in DCM to afford (2S)-2-[[8-[3-[3-[5-[[tert-butoxycarbonyl-[[(2S)-5-oxopyrrolidin-2-yl]methyl]amino]methyl]-6-methoxy-2-pyridyl]-2-chloro-phenyl]-2-chloro-phenyl]-4-oxo-pyrido[1,2-a]pyrimidin-3-yl]methylamino]-3-hydroxy-propanoate (19.4 mg, 21% yield) as a yellow oil. MS: m/z found 831.35, 833 [M+H]⁺.

Methyl (2S)-2-[[8-[3-[3-[5-[[tert-butoxycarbonyl-[[(2S)-5-oxopyrrolidin-2-yl]methyl]amino]methyl]-6-methoxy-2-pyridyl]-2-chloro-phenyl]-2-chloro-phenyl]-4-oxo-pyrido[1,2-a]pyrimidin-3-yl]methylamino]-3-hydroxy-propanoate (19. mg, 0.02 mmol) was dissolved in 0.5 mL DCM and 4M HCl solution in dioxane (29 μL, 4 mg, 0.12 mmol) was added. The reaction was stirred at rt for 20 minutes and the mixture was subsequently concentrated. Crude sample was purified by reverse phase HPLC to afford methyl (2S)-2-[[8-[2-chloro-3-[2-chloro-3-[6-methoxy-5-[[[(2S)-5-oxopyrrolidin-2-yl]methylamino]methyl]-2-pyridyl]phenyl]phenyl]-4-oxo-pyrido[1,2-a]pyrimidin-3-yl]methylamino]-3-hydroxy-propanoate (14.7 mg, 86.1% yield) as a clear film. LC/MS consistent with desired compound. MS: m/z found 731.5, 733.5 [M+H]⁺. ¹H NMR (400 MHz, Chloroform-d) δ 9.09 (d, J=7.4 Hz, 1H), 8.34 (s, 1H), 7.76-7.73 (m, 1H), 7.69 (d, J=7.7 Hz, 1H), 7.59 (d, J=7.4 Hz, 1H), 7.46 (dd, J=14.5, 6.1 Hz, 4H), 7.35 (dd, J=15.5, 7.3 Hz, 2H), 7.28 (s, 1H), 6.04 (s, 1H), 4.03 (s, 3H), 3.91 (d, J=10.6 Hz, 3H), 3.81 (s, 2H), 3.73 (s, 4H), 3.54 (s, 1H), 3.22 (s, 1H), 2.80 (d, J=11.7 Hz, 1H), 2.57 (t, J=10.3 Hz, 1H), 2.36 (t, J=8.2 Hz, 2H), 2.24 (dd, J=13.3, 6.9 Hz, 1H).

### Example 102: (S)-8-(2,2′-Dichloro-3′-(5-(((2-hydroxyethyl)amino)methyl)-6-methoxypyridin-2-yl)-[1,1′-biphenyl]-3-yl)-3-((((5-oxopyrrolidin-2-yl)methyl)amino)methyl)-4H-pyrido[1,2-a]pyrimidin-4-one

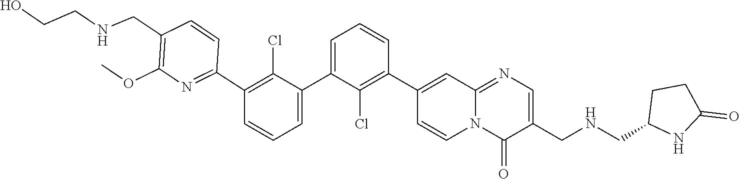 [SMILES: COc1nc(-c2cccc(-c3cccc(-c4ccn5c(=O)c(CNC[C@@H]6CCC(=O)N6)cnc5c4)c3Cl)c2Cl)ccc1CNCCO]

### (S)-8-(3-Bromo-2-chlorophenyl)-3-((((5-oxopyrrolidin-2-yl)-methyl)-amino)-methyl)-4H-pyrido[1,2-a]-pyrimidin-4-one

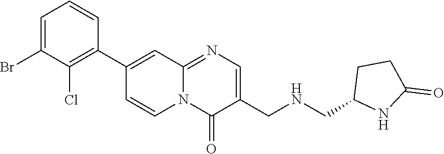 [SMILES: O=C1CC[C@@H](CNCc2cnc3cc(-c4cccc(Br)c4Cl)ccn3c2=O)N1]

8-(3-Bromo-2-chloro-phenyl)-4-oxo-pyrido[1,2-a]pyrimidine-3-carbaldehyde (400 mg, 1.10 mmol), (5S)-5-(amino-methyl)pyrrolidin-2-one (176 mg, 1.54 mmol) and acetic acid (86 mg, 1.43 mmol) were dissolved in 10 ml of 1:1 MeOH/THF solvent mix, and the reaction was stirred at 25° C. for 3 hours. NaBH₃CN (104 mg, 1.65 mmol) was added portion-wise, and the resulting mixture was stirred at 25° C. for 1 hour. The reaction was concentrated and the crude residue was diluted with 20 ml of water, then extracted with DCM (3×20 ml). The combined organics were further washed with brine, dried over sodium sulfate, filtered and concentrated to afford 8-(3-bromo-2-chloro-phenyl)-3-[[[(2S)-5-oxopyrrolidin-2-yl]methylamino]methyl]pyrido[1,2-a]pyrimidin-4-one (480 mg) as an orange foam. Material was used as—is for the next step. MS: m/z found 461.1, 463.1 [M+H]⁺.

### tert-Butyl (S)-((8-(3-bromo-2-chlorophenyl)-4-oxo-4H-pyrido[1,2-a]-pyrimidin-3-yl)-methyl)-((5-oxopyrrolidin-2-yl)-methyl)-carbamate

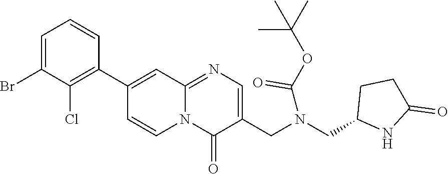 [SMILES: CC(C)(C)OC(=O)N(Cc1cnc2cc(-c3cccc(Br)c3Cl)ccn2c1=O)C[C@@H]1CCC(=O)N1]

8-(3-Bromo-2-chloro-phenyl)-3-[[[(2S)-5-oxopyrrolidin-2-yl]methylamino]methyl]pyrido[1,2-a]pyrimidin-4-one (0.48 g, 1.04 mmol) and DIEA (0.72 mL, 0.54 g, 4.16 mmol) were dissolved in 8 mL THE and di-tert-butyl dicarbonate (0.45 g mg, 2.08 mmol) was added portion-wise. The resulting mixture was stirred at rt for 12 hours. DCM (20 ml) was added to the reaction and the solution was washed with aqueous saturated sodium bicarbonate solution (10 mL×2) and brine (15 mL×2). The organic layer was concentrated, and the crude sample was purified by silica gel chromatography, eluting with 0-10% MeOH gradient in DCM to afford tert-butyl N-[[8-(3-bromo-2-chloro-phenyl)-4-oxo-pyrido[1,2-a]pyrimidin-3-yl]methyl]-N-[[(2S)-5-oxopyrrolidin-2-yl]methyl]carbamate (432.00 mg, 74.0% yield) as an orange foam. MS: m/z found 561.2, 563.2 [M+H]⁺.

### tert-Butyl (S)-((8-(2,2′-dichloro-3′-(5-formyl-6-methoxypyridin-2-yl)-[1,1′-biphenyl]-3-yl)-4-oxo-4H-pyrido[1,2-a]pyrimidin-3-yl)methyl)((5-oxopyrrolidin-2-yl)methyl)carbamate

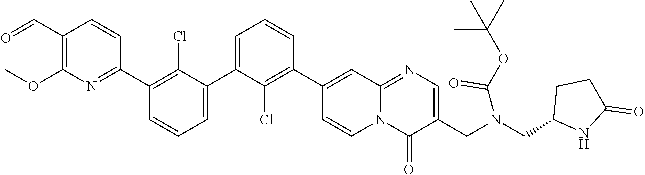 [SMILES: COc1nc(-c2cccc(-c3cccc(-c4ccn5c(=O)c(CN(C[C@@H]6CCC(=O)N6)C(=O)OC(C)(C)C)cnc5c4)c3Cl)c2Cl)ccc1C=O]

Pd(PPh₃)₄ (23 mg, 0.02 mmol), potassium carbonate (55 mg, 0.40 mmol), tert-butyl N-[[8-(3-bromo-2-chloro-phenyl)-4-oxo-pyrido[1,2-a]pyrimidin-3-yl]methyl]-N-[[(2S)-5-oxopyrrolidin-2-yl]methyl]carbamate (75 mg, 0.13 mmol) and 6-[2-chloro-3-(4,4,5,5-tetramethyl-1,3,2-dioxaborolan-2-yl)phenyl]-2-methoxy-pyridine-3-carbaldehyde (50 mg, 0.13 mmol) were suspended in 2 mL of 4:1 dioxane/water solvent mix, and the reaction was heated at 105° C. for 20 minutes. The solution was subsequently cooled to rt, diluted with 5 mL water, and extracted with EtOAc (3×3 mL). The combined organics were concentrated and crude product was purified by silica gel chromatography, eluting with 0-5% MeOH gradient in DCM to afford tert-butyl N-[[8-[2-chloro-3-[2-chloro-3-(5-formyl-6-methoxy-2-pyridyl)phenyl]phenyl]-4-oxo-pyrido[1,2-a]pyrimidin-3-yl]methyl]-N-[[(2S)-5-oxopyrrolidin-2-yl]methyl]carbamate (79 mg, 81.0% yield) as a tan foam. MS: m/z found 728.3, 730.3 [M+H]⁺.

### tert-Butyl (S)-((8-(2,2′-dichloro-3′-(5-(((2-hydroxyethyl)amino)methyl)-6-methoxypyridin-2-yl)-[1,1′-biphenyl]-3-yl)-4-oxo-4H-pyrido[1,2-a]pyrimidin-3-yl)methyl)((5-oxopyrrolidin-2-yl)methyl)carbamate

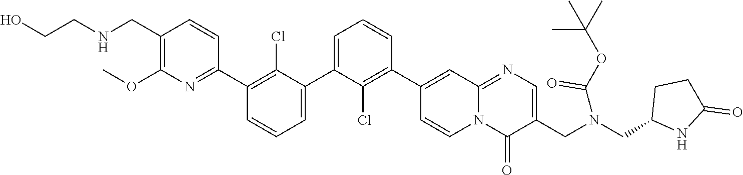 [SMILES: COc1nc(-c2cccc(-c3cccc(-c4ccn5c(=O)c(CN(C[C@@H]6CCC(=O)N6)C(=O)OC(C)(C)C)cnc5c4)c3Cl)c2Cl)ccc1CNCCO]

tert-Butyl N-[[8-[2-chloro-3-[2-chloro-3-(5-formyl-6-methoxy-2-pyridyl)phenyl]phenyl]-4-oxo-pyrido[1,2-a]pyrimidin-3-yl]methyl]-N-[[(2S)-5-oxopyrrolidin-2-yl]methyl]carbamate (26 mg, 0.04 mmol), 2-aminoethanol (6.5 mg, 0.11 mmol) and acetic acid (4 mg, 0.07 mmol) were dissolved in 1 mL of 1:1 MeOH/THF solvent mix, and the reaction was stirred at 25° C. for 3 hours. NaBH₃CN (4.5 mg, 0.07 mmol) was added portion-wise, and the resulting mixture was stirred at 25° C. for 1 hour. The reaction was concentrated and the crude residue was dissolved in 2 ml water, then extracted with DCM (3×2 mL). The combined organics were washed with water (3 ml), brine (3 ml), dried over sodium sulfate, then filtered and concentrated. The crude material was purified by silica gel chromatography, eluting with 0-18% MeOH in DCM to afford tert-butyl N-[[8-[2-chloro-3-[2-chloro-3-[5-[(2-hydroxyethylamino)methyl]-6-methoxy-2-pyridyl]phenyl]phenyl]-4-oxo-pyrido[1,2-a]pyrimidin-3-yl]methyl]-N-[[(2S)-5-oxopyrrolidin-2-yl]methyl]carbamate (14 mg, 50.7% yield) as a clear oil. MS: m/z found 773.5, 775.6 [M+H]⁺.

### (S)-8-(2,2′-Dichloro-3′-(5-(((2-hydroxyethyl)amino)methyl)-6-methoxypyridin-2-yl)-[1,1′-biphenyl]-3-yl)-3-((((5-oxopyrrolidin-2-yl)methyl)amino)methyl)-4H-pyrido[1,2-a]pyrimidin-4-one

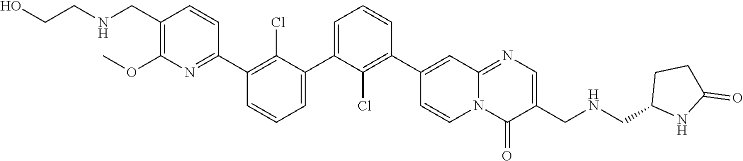 [SMILES: COc1nc(-c2cccc(-c3cccc(-c4ccn5c(=O)c(CNC[C@@H]6CCC(=O)N6)cnc5c4)c3Cl)c2Cl)ccc1CNCCO]

tert-Butyl N-[[8-[2-chloro-3-[2-chloro-3-[5-[(2-hydroxyethylamino)methyl]-6-methoxy-2-pyridyl]phenyl]phenyl]-4-oxo-pyrido[1,2-a]pyrimidin-3-yl]methyl]-N-[[(2S)-5-oxopyrrolidin-2-yl]methyl]carbamate (14 mg, 0.02 mmol) was dissolved in 0.5 ml DCM and 4M hydrogen chloride solution in dioxane (18 μL, 0.07 mmol) was added. The reaction was stirred at rt for 20 minutes and the precipitate was filtered and further washed with DCM (2×2 mL). The resulting solid was dissolved in 1 ml water and the pH was adjusted to pH=9 by slow addition of aqueous saturated sodium bicarbonate solution. The aqueous was extracted with DCM (3×1 mL) and the combined organics were washed with brine, dried over sodium sulfate, filtered and concentrated to afford (S)-8-(2,2′-dichloro-3′-(5-(((2-hydroxyethyl)amino)methyl)-6-methoxypyridin-2-yl)-[1,1′-biphenyl]-3-yl)-3-((((5-oxopyrrolidin-2-yl)methyl)amino)methyl)-4H-pyrido[1,2-a]pyrimidin-4-one (10.1 mg, 82.9% yield) as a clear film. MS: m/z found 673.2, 675.2 [M+H]⁺. ¹H NMR (400 MHz, Chloroform-d) δ 9.08 (d, J=7.4 Hz, 1H), 8.32 (s, 1H), 7.76 (d, J=1.8 Hz, 1H), 7.69 (dd, J=7.7, 1.7 Hz, 1H), 7.61 (d, J=7.4 Hz, 1H), 7.52-7.42 (m, 4H), 7.38 (dd, J=7.5, 1.9 Hz, 1H), 7.33 (dd, J=7.5, 1.7 Hz, 1H), 7.27 (s, 1H), 6.19 (s, 1H), 4.03 (s, 3H), 3.90-3.83 (m, 2H), 3.82 (s, 2H), 3.78 (s, 1H), 3.70 (t, J=5.2 Hz, 2H), 2.86-2.76 (m, 3H), 2.56 (dd, J=12.0, 8.8 Hz, 1H), 2.35 (t, J=8.1 Hz, 2H), 2.29-2.18 (m, 1H), 1.80-1.70 (m, 1H).

### Example 103: (S)—N-(2-(((6-(2,2′-Dichloro-3′-(4-oxo-3-((((5-oxopyrrolidin-2-yl)methyl)amino)methyl)-4H-pyrido[1,2-a]pyrimidin-8-yl)-[1,1′-biphenyl]-3-yl)-2-methoxypyridin-3-yl)methyl)amino)ethyl)acetamide

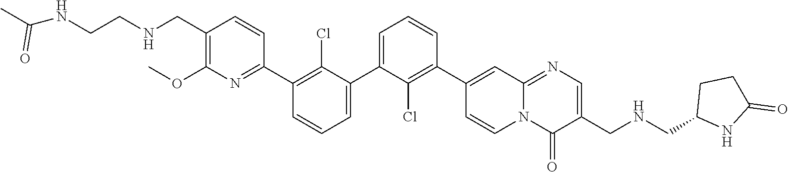 [SMILES: COc1nc(-c2cccc(-c3cccc(-c4ccn5c(=O)c(CNC[C@@H]6CCC(=O)N6)cnc5c4)c3Cl)c2Cl)ccc1CNCCNC(C)=O]

tert-Butyl N-[[8-[2-chloro-3-[2-chloro-3-(5-formyl-6-methoxy-2-pyridyl)phenyl]phenyl]-4-oxo-pyrido[1,2-a]pyrimidin-3-yl]methyl]-N-[[(2S)-5-oxopyrrolidin-2-yl]methyl]carbamate (26 mg, 0.04 mmol), N-(2-aminoethyl)acetamide (11 mg, 0.11 mmol) and acetic acid (4 mg, 0.07 mmol were dissolved in 1 ml of 1:1 MeOH/THF solvent mix, and the reaction was stirred at 25° C. for 3 hours. NaBH₃CN (4.5 mg, 0.07 mmol) was added portion-wise, and the resulting mixture was stirred at 25° C. for 1 hour. The reaction was concentrated and the residue was dissolved in water (3 mL), then extracted with DCM (3×2 mL). The combined organics were further washed with brine, dried over sodium sulfate, then filtered and concentrated. The crude material was purified by silica column, eluting with 0-10% MeOH in DCM to afford tert-butyl N-[[8-[3-[3-[5-[(2-acetamidoethylamino)methyl]-6-methoxy-2-pyridyl]-2-chloro-phenyl]-2-chloro-phenyl]-4-oxo-pyrido[1,2-a]pyrimidin-3-yl]methyl]-N-[[(2S)-5-oxopyrrolidin-2-yl]methyl]carbamate (12 mg, 41.3% yield) as an orange oil. MS: m/z found 814.4, 816.4 [M+H]⁺.

tert-butyl N-[[8-[3-[3-[5-[(2-acetamidoethylamino)methyl]-6-methoxy-2-pyridyl]-2-chloro-phenyl]-2-chloro-phenyl]-4-oxo-pyrido[1,2-a]pyrimidin-3-yl]methyl]-N-[[(2S)-5-oxopyrrolidin-2-yl]methyl]carbamate (12 mg, 0.01 mmol) was dissolved in 0.5 ml DCM and 4M HCl solution in dioxane (15 μL, 0.06 mmol) was added. The reaction was stirred at rt for 20 minutes and the mixture was subsequently concentrated. The crude sample was purified by reverse phase HPLC to afford N-[2-[[6-[2-chloro-3-[2-chloro-3-[4-oxo-3-[[[(2S)-5-oxopyrrolidin-2-yl]methylamino]methyl]pyrido[1,2-a]pyrimidin-8-yl]phenyl]phenyl]-2-methoxy-3-pyridyl]methylamino]ethyl]acetamide (8.1 mg, 77.0% yield) as a clear film. MS: m/z found 714.3, 716.3 [M+H]⁺. 1H NMR (400 MHz, Chloroform-d) δ 9.08 (d, J=7.4 Hz, 1H), 8.32 (s, 1H), 7.76 (d, J=1.8 Hz, 1H), 7.69 (dd, J=7.7, 1.7 Hz, 1H), 7.58 (d, J=7.4 Hz, 1H), 7.46 (ddt, J=11.0, 7.9, 4.4 Hz, 4H), 7.38 (dd, J=7.4, 1.9 Hz, 1H), 7.33 (dd, J=7.6, 1.8 Hz, 1H), 7.27 (s, 1H), 6.19 (s, 1H), 6.08 (s, 1H), 4.03 (s, 3H), 3.91-3.81 (m, 2H), 3.79 (s, 3H), 3.38 (q, J=5.6 Hz, 2H), 2.80 (dt, J=11.3, 5.1 Hz, 3H), 2.56 (dd, J=12.0, 8.8 Hz, 1H), 2.35 (t, J=8.0 Hz, 2H), 2.30-2.16 (m, 1H), 2.00 (s, 3H), 1.78-1.70 (m, 1H).

### Example 104: 8-(2,2′-Dichloro-3″-fluoro-5″-methoxy-4″-(((((S)-5-oxopyrrolidin-2-yl)methyl)amino)methyl)-[1,1′:3′,1″-terphenyl]-3-yl)-3-(((((S)-5-oxopyrrolidin-2-yl)methyl)amino)methyl)-4H-pyrido[1,2-a]pyrimidin-4-one

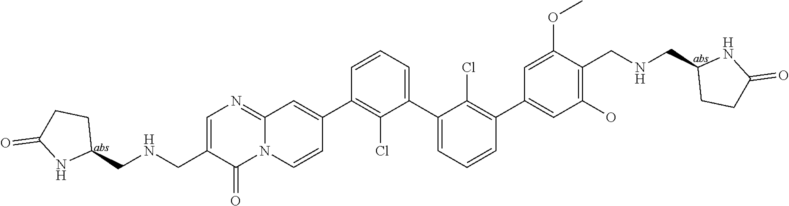 [SMILES: COc1cc(-c2cccc(-c3cccc(-c4ccn5c(=O)c(CNC[C@@H]6CCC(=O)N6)cnc5c4)c3Cl)c2Cl)cc(O)c1CNC[C@@H]1CCC(=O)N1]

### 8-[2-Chloro-3-[2-chloro-3-(3-fluoro-4-formyl-5-methoxy-phenyl)phenyl]phenyl]-4-oxo-pyrido[1,2-a]pyrimidine-3-carbaldehyde

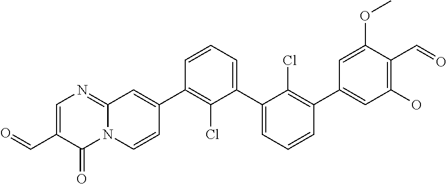 [SMILES: COc1cc(-c2cccc(-c3cccc(-c4ccn5c(=O)c(C=O)cnc5c4)c3Cl)c2Cl)cc(O)c1C=O]

To a solution of 8-[2-chloro-3-[2-chloro-3-(4,4,5,5-tetramethyl-1,3,2-dioxaborolan-2-yl)phenyl]phenyl]-4-oxo-pyrido[1,2-a]pyrimidine-3-carbaldehyde (30 mg, 0.06 mmol), 4-bromo-2-fluoro-6-methoxy-benzaldehyde (20 mg, 0.09 mmol) and potassium carbonate (24 mg, 0.17 mmol) in degassed dioxane/water (6:1) was added Pd(PPh₃)₄ (7 mg, 0.01 mmol) and the mixture was stirred at 90° C. for 4 hours under N₂(g). Water was added and the mixture was extracted into EtOAc three times. The combined organic layers were dried over anhydrous sodium sulfate, concentrated, and purified on silica gel column using EtOAc in hexanes (0 to 60% gradient) to provide 8-[2-chloro-3-[2-chloro-3-(3-fluoro-4-formyl-5-methoxy-phenyl)phenyl]phenyl]-4-oxo-pyrido[1,2-a]pyrimidine-3-carbaldehyde (16 mg, 50.8%). MS: m/z found 547.1 [M+H]⁺.

### 8-[2-Chloro-3-[2-chloro-3-[3-fluoro-5-methoxy-4-[[[(2S)-5-oxopyrrolidin-2-yl]methylamino]methyl]phenyl]phenyl]phenyl]-3-[[[(2S)-5-oxopyrrolidin-2-yl]methylamino]methyl]pyrido[1,2-a]pyrimidin-4-one

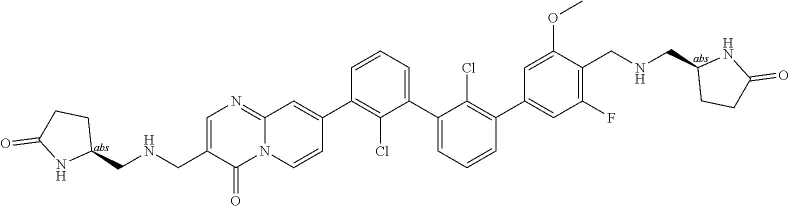 [SMILES: COc1cc(-c2cccc(-c3cccc(-c4ccn5c(=O)c(CNC[C@@H]6CCC(=O)N6)cnc5c4)c3Cl)c2Cl)cc(F)c1CNC[C@@H]1CCC(=O)N1]

A mixture of 8-[2-chloro-3-[2-chloro-3-(3-fluoro-4-formyl-5-methoxy-phenyl)phenyl]phenyl]-4-oxo-pyrido[1,2-a]pyrimidine-3-carbaldehyde (36 mg, 0.07 mmol), (5S)-5-(aminomethyl)pyrrolidin-2-one (30 mg, 0.26 mmol) and acetic acid (7.5 uL, 8 mg, 0.13 mmol) in MeOH/THF (1:1) was stirred for 1.5 hour at rt. NaBH₃CN (16.5 mg, 0.26 mmol) was added, the mixture was stirred for additional 5 h, quenched with water and directly purified by HPLC. The collected fraction was neutralized with sat. NaHCO₃(aq.), acetonitrile was evaporated, and aqueous phase was extracted into DCM three times. The combined organic extracts were dried over anhydrous magnesium sulfate and concentrated in vacuo to give 8-[2-chloro-3-[2-chloro-3-[3-fluoro-5-methoxy-4-[[[(2S)-5-oxopyrrolidin-2-yl]methylamino]methyl]phenyl]phenyl]phenyl]-3-[[[(2S)-5-oxopyrrolidin-2-yl]methylamino]methyl]pyrido[1,2-a]pyrimidin-4-one (33 mg, 67.5%). MS: m/z found 743.2 [M+H]⁺; ¹H NMR (400 MHz, Methanol-d₄) δ 9.13 (dd, J=7.4, 0.8 Hz, 1H), 8.41 (d, J=0.6 Hz, 1H), 7.76 (dd, J=1.9, 0.8 Hz, 1H), 7.65-7.57 (m, 2H), 7.54-7.50 (m, 2H), 7.50-7.45 (m, 2H), 7.39 (dd, J=6.9, 2.4 Hz, 1H), 6.91 (d, J=1.2 Hz, 1H), 6.84 (dd, J=9.8, 1.5 Hz, 1H), 3.92 (s, 3H), 3.92-3.90 (m, 2H), 3.88-3.80 (m, 4H), 2.79-2.62 (m, 4H), 2.37-2.23 (m, 6H), 1.88-1.73 (m, 2H).

### Example 105: 8-[2-Chloro-3-[2-chloro-3-[2-fluoro-3-methoxy-4-[[[(2S)-5-oxopyrrolidin-2-yl]methylamino]methyl]phenyl]phenyl]phenyl]-3-[[[(2S)-5-oxopyrrolidin-2-yl]methylamino]methyl]pyrido[1,2-a]pyrimidin-4-one

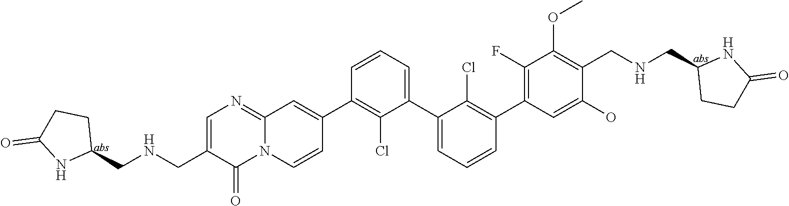 [SMILES: COc1c(F)c(-c2cccc(-c3cccc(-c4ccn5c(=O)c(CNC[C@@H]6CCC(=O)N6)cnc5c4)c3Cl)c2Cl)cc(O)c1CNC[C@@H]1CCC(=O)N1]

### 8-[2-Chloro-3-[2-chloro-3-(2-fluoro-4-formyl-3-methoxy-phenyl)phenyl]phenyl]-4-oxo-pyrido[1,2-a]pyrimidine-3-carbaldehyde

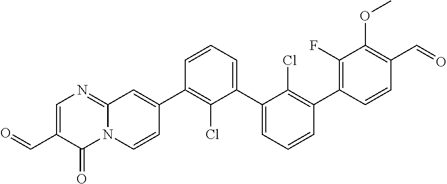 [SMILES: COc1c(C=O)ccc(-c2cccc(-c3cccc(-c4ccn5c(=O)c(C=O)cnc5c4)c3Cl)c2Cl)c1F]

To a solution of 8-[2-chloro-3-[2-chloro-3-(4,4,5,5-tetramethyl-1,3,2-dioxaborolan-2-yl)phenyl]phenyl]-4-oxo-pyrido[1,2-a]pyrimidine-3-carbaldehyde (50 mg, 0.10 mmol), 4-bromo-3-fluoro-2-methoxy-benzaldehyde (29 mg, 0.12 mmol) and potassium carbonate (40 mg, 0.29 mmol) in degassed dioxane/water (6:1) was added Pd(PPh₃)₄ (11 mg, 0.01 mmol) and the mixture was stirred at 90° C. for 4 hours under N₂(g). Water was added and the mixture was extracted into EtOAc three times. The combined organic layers were dried over anhydrous sodium sulfate, concentrated, and purified on silica gel column using EtOAc in hexanes (0 to 60% gradient) to give 8-[2-chloro-3-[2-chloro-3-(2-fluoro-4-formyl-3-methoxy-phenyl)phenyl]phenyl]-4-oxo-pyrido[1,2-a]pyrimidine-3-carbaldehyde (15 mg, 28.6%). MS: m/z found 547.1 [M+H]⁺.

### 8-[2-Chloro-3-[2-chloro-3-[2-fluoro-3-methoxy-4-[[[(2S)-5-oxopyrrolidin-2-yl]methylamino]methyl]phenyl]phenyl]phenyl]-3-[[[(2S)-5-oxopyrrolidin-2-yl]methylamino]methyl]pyrido[1,2-a]pyrimidin-4-one

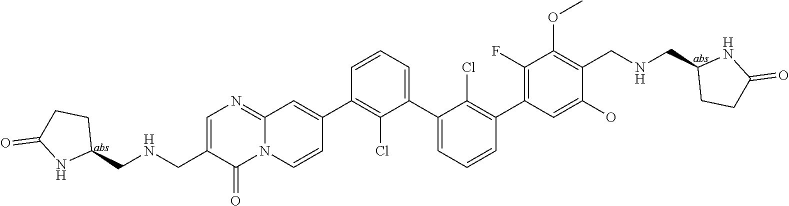 [SMILES: COc1c(F)c(-c2cccc(-c3cccc(-c4ccn5c(=O)c(CNC[C@@H]6CCC(=O)N6)cnc5c4)c3Cl)c2Cl)cc(O)c1CNC[C@@H]1CCC(=O)N1]

A mixture of 8-[2-chloro-3-[2-chloro-3-(2-fluoro-4-formyl-3-methoxy-phenyl)phenyl]phenyl]-4-oxo-pyrido[1,2-a]pyrimidine-3-carbaldehyde (30 mg, 0.05 mmol), (5S)-5-(aminomethyl)pyrrolidin-2-one (25 mg, 0.22 mmol) and acetic acid (6 uL, 6.5 mg, 0.11 mmol) in MeOH/THF (1:1) was stirred for 1.5 hour at rt. NaBH₃CN (14 mg, 0.22 mmol) was added, the mixture was stirred for additional 5 hrs, quenched with water and directly purified by HPLC. The collected fraction was neutralized with sat. NaHCO₃(aq.), acetonitrile was evaporated, and aqueous phase was extracted into DCM three times. The combined organic extracts were dried over anhydrous magnesium sulfate and concentrated in vacuo to give 8-[2-chloro-3-[2-chloro-3-[2-fluoro-3-methoxy-4-[[[(2S)-5-oxopyrrolidin-2-yl]methylamino]methyl]phenyl]phenyl]phenyl]-3-[[[(2S)-5-oxopyrrolidin-2-yl]methylamino]methyl]pyrido[1,2-a]pyrimidin-4-one (25 mg, 61.3%). MS: m/z found 743.2 [M+H]⁺; ¹H NMR (400 MHz, Methanol-d₄) δ 9.14 (dd, J=7.4, 0.7 Hz, 1H), 8.42 (s, 1H), 7.77 (dd, J=1.9, 0.7 Hz, 1H), 7.65-7.46 (m, 5H), 7.45-7.40 (m, 2H), 7.24 (dd, J=7.9, 1.3 Hz, 1H), 7.09-6.99 (m, 1H), 3.97 (d, J=1.8 Hz, 3H), 3.94-3.79 (m, 6H), 2.81-2.64 (m, 4H), 2.41-2.21 (m, 6H), 1.88-1.75 (m, 2H).

### Example 106: 8-(2,2′-Dichloro-2″-fluoro-5″-methoxy-4″-(((((S)-5-oxopyrrolidin-2-yl)methyl)amino)methyl)-[1,1′:3′,1″-terphenyl]-3-yl)-3-(((((S)-5-oxopyrrolidin-2-yl)methyl)amino)methyl)-4H-pyrido[1,2-a]pyrimidin-4-one

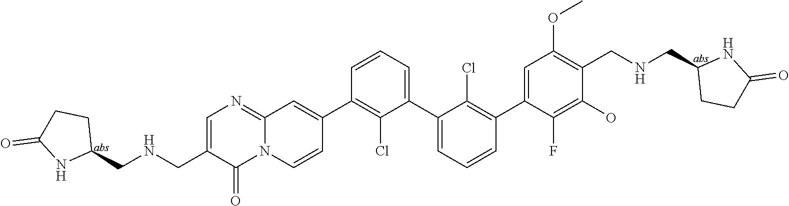 [SMILES: COc1cc(-c2cccc(-c3cccc(-c4ccn5c(=O)c(CNC[C@@H]6CCC(=O)N6)cnc5c4)c3Cl)c2Cl)c(F)c(O)c1CNC[C@@H]1CCC(=O)N1]

### 8-[2-Chloro-3-[2-chloro-3-(2-fluoro-4-formyl-5-methoxy-phenyl)phenyl]phenyl]-4-oxo-pyrido[1,2-a]pyrimidine-3-carbaldehyde

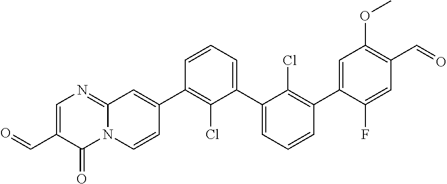 [SMILES: COc1cc(-c2cccc(-c3cccc(-c4ccn5c(=O)c(C=O)cnc5c4)c3Cl)c2Cl)c(F)cc1C=O]

To a solution of 8-[2-chloro-3-[2-chloro-3-(4,4,5,5-tetramethyl-1,3,2-dioxaborolan-2-yl)phenyl]phenyl]-4-oxo-pyrido[1,2-a]pyrimidine-3-carbaldehyde (60 mg, 0.12 mmol), 4-bromo-5-fluoro-2-methoxy-benzaldehyde (35 mg, 0.15 mmol) and potassium carbonate (48 mg, 0.35 mmol) in degassed dioxane/water (6:1) was added Pd(PPh₃)₄ (13 mg, 0.01 mmol) and the mixture was stirred at 90° C. for 4 hours under N₂(g). Water was added and the mixture was extracted into EtOAc three times. The combined organic layers were dried over anhydrous sodium sulfate, concentrated, and purified on silica gel column using EtOAc in hexanes (0 to 60% gradient) to give 8-[2-chloro-3-[2-chloro-3-(2-fluoro-4-formyl-5-methoxy-phenyl)phenyl]phenyl]-4-oxo-pyrido[1,2-a]pyrimidine-3-carbaldehyde (28 mg, 44.4%). MS: m/z found 547.1 [M+H]⁺.

### 8-[2-Chloro-3-[2-chloro-3-[2-fluoro-5-methoxy-4-[[[(2S)-5-oxopyrrolidin-2-yl]methylamino]methyl]phenyl]phenyl]phenyl]-3-[[[(2S)-5-oxopyrrolidin-2-yl]methylamino]methyl]pyrido[1,2-a]pyrimidin-4-one

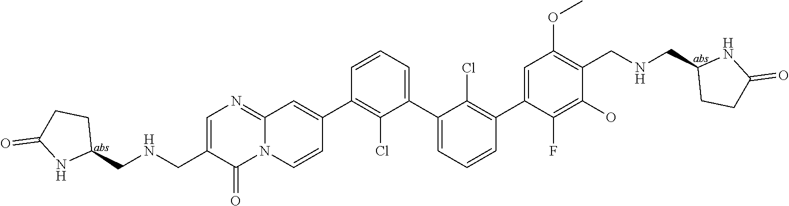 [SMILES: COc1cc(-c2cccc(-c3cccc(-c4ccn5c(=O)c(CNC[C@@H]6CCC(=O)N6)cnc5c4)c3Cl)c2Cl)c(F)c(O)c1CNC[C@@H]1CCC(=O)N1]

A mixture of 8-[2-chloro-3-[2-chloro-3-(2-fluoro-4-formyl-5-methoxy-phenyl)phenyl]phenyl]-4-oxo-pyrido[1,2-a]pyrimidine-3-carbaldehyde (23 mg, 0.04 mmol), (5S)-5-(aminomethyl)pyrrolidin-2-one (19 mg, 0.17 mmol) and acetic acid (4.81 uL, 5. mg, 0.08 mmol) in MeOH/THF (1:1) was stirred for 1.5 hour at rt. NaBH₃CN (10.56 mg, 0.17 mmol) was added, the mixture was stirred for additional 5 h, quenched with water and directly purified by HPLC. The collected fraction was neutralized with sat. NaHCO₃(aq.), acetonitrile was evaporated, and aqueous phase was extracted into DCM three times. The combined organic extracts were dried over anhydrous magnesium sulfate and concentrated in vacuo to give 8-[2-chloro-3-[2-chloro-3-[2-fluoro-5-methoxy-4-[[[(2S)-5-oxopyrrolidin-2-yl]methylamino]methyl]phenyl]phenyl]phenyl]-3-[[[(2S)-5-oxopyrrolidin-2-yl]methylamino]methyl]pyrido[1,2-a]pyrimidin-4-one (21 mg, 67.2%). MS: m/z found 743.2 [M+H]⁺; ¹H NMR (400 MHz, Methanol-d₄) δ 9.13 (dd, J=7.4, 0.7 Hz, 1H), 8.41 (s, 1H), 7.76 (dd, J=1.9, 0.7 Hz, 1H), 7.64-7.57 (m, 2H), 7.55-7.47 (m, 3H), 7.47-7.39 (m, 2H), 7.18 (d, J=9.7 Hz, 1H), 6.93 (d, J=5.9 Hz, 1H), 3.92-3.77 (m, 9H), 2.79-2.63 (m, 4H), 2.37-2.21 (m, 6H), 1.82 (dddd, J=13.1, 12.2, 8.8, 5.5 Hz, 2H).

### Example 107: 8-(2,2′-Dichloro-3′-(6-methoxy-5-(((((S)-5-oxopyrrolidin-2-yl)methyl)amino)methyl)pyrazin-2-yl)-[1,1′-biphenyl]-3-yl)-3-(((((S)-5-oxopyrrolidin-2-yl)methyl)amino)methyl)-4H-quinolizin-4-one

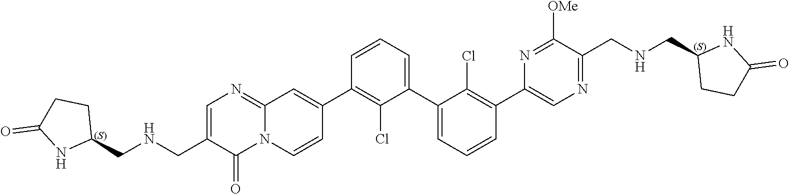

### 5-(2-Dhloro-3-(4,4,5,5-tetramethyl-1,3,2-dioxaborolan-2-yl)phenyl)-3-methoxypyrazine-2-carbaldehyde

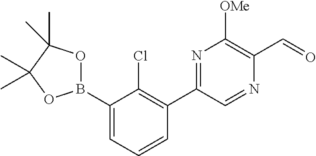 [SMILES: COc1nc(-c2cccc(B3OC(C)(C)C(C)(C)O3)c2Cl)cnc1C=O]

To a mixture 5-(3-bromo-2-chlorophenyl)-3-methoxypyrazine-2-carbaldehyde (0.5 g, 1.53 mmol) in dioxane (20 mL) was added 4,4,4′,4′,5,5,5′,5′-octamethyl-2,2′-bi(1,3,2-dioxaborolane (0.81 g, 3.21 mmol), 1,1′-bis(diphenylphosphino)ferrocene-palladium(II) dichloride dichloromethane complex (0.12 g, 0.15 mmol) and passium acetate (0.45 g, 4.58 mmol). The mixture was stirred at 95° C. for 15 hr. The reaction mixture was filtered and the filtrate was concentrated under reduced pressure. The residue was purified by normal phase SiO₂ chromatography (0-100% EtOAc/petroleum ether) to afford 5-(2-chloro-3-(4,4,5,5-tetramethyl-1,3,2-dioxaborolan-2-yl)phenyl)-3-methoxypyrazine-2-carbaldehyde as a yellow solid (0.14 g, 24% yield). ¹H NMR (400 MHz, DMSO-d₆): δ 10.18 (s, 1H), 8.74 (s, 1H), 7.80-7.77 (m, 2H), 7.56 (t, J=7.6 Hz, 1H), 4.07 (s, 3H), 1.34 (s, 12H).

### 8-(2,2′-Dichloro-3′-(5-formyl-6-methoxypyrazin-2-yl)-[1,1′-biphenyl]-3-yl)-4-oxo-4H-quinolizine-3-carbaldehyde

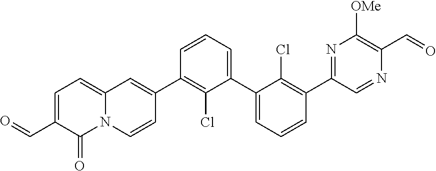 [SMILES: COc1nc(-c2cccc(-c3cccc(-c4ccn5c(=O)c(C=O)ccc5c4)c3Cl)c2Cl)cnc1C=O]

To a mixture 5-(2-chloro-3-(4,4,5,5-tetramethyl-1,3,2-dioxaborolan-2-yl)phenyl)-3-methoxypyrazine-2-carbaldehyde (0.14 g, 0.37 mmol) in dioxane/H₂O (4:1, 5 mL) was added 8-(3-bromo-2-chlorophenyl)-4-oxo-4H-quinolizine-3-carbaldehyde (0.16 g, 0.45 mmol), [1,1′-Bis(di-tert-butylphosphino)ferrocene]dichloropalladium(II) (0.02 g, 0.04 mmol) and potassium phosphate (0.16 g, 0.75 mmol). The reaction mixture was stirred at 110° C. for 0.5 hr. To the reaction mixture was added water (30 mL), then extracted with EtOAc (3×30 ml). The combined organic layers were washed with saturated aqueous brine solution (20 mL), dried over anhydrous sodium sulfate, filtered and concentrated under reduced pressure to give a residue. The residue was purified by reverse phase HPLC to afford 8-(2,2′-dichloro-3′-(5-formyl-6-methoxypyrazin-2-yl)-[1,1′-biphenyl]-3-yl)-4-oxo-4H-quinolizine-3-carbaldehyde as a yellow solid (0.2 g, crude). m/z found: 530.0 [M+H]⁺. 8-(2,2′-Dichloro-3′-(6-methoxy-5-(((((S)-5-oxopyrrolidin-2-yl)methyl)amino)methyl)pyrazin-2-yl)-[1,1′-biphenyl]-3-yl)-3-(((((S)-5-oxopyrrolidin-2-yl)methyl)amino)methyl)-4H-quinolizin-4-one

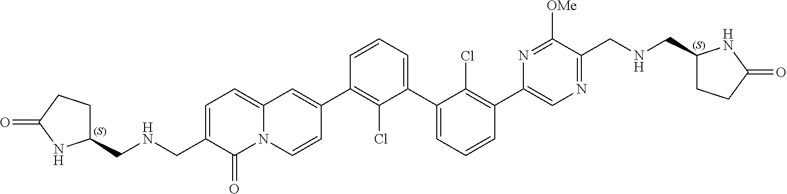

To a mixture 8-(2,2′-dichloro-3′-(5-formyl-6-methoxypyrazin-2-yl)-[1,1′-biphenyl]-3-yl)-4-oxo-4H-quinolizine-3-carbaldehyde (0.2 g, 0.38 mmol) in THF/DCM (1:1, 6 mL) was added (S)-5-(aminomethyl)pyrrolidin-2-one as a hydrochloride salt (0.34 g, 2.26 mmol) and sodium acetate (0.31 g, 3.77 mmol). The mixture was stirred at r.t. for 10 hr. Sodium triacetoxyborohydride (0.48 g, 2.26 mmol) was added and the mixture was stirred at r.t. for 2 hr. The reaction mixture was filtered and the filtrate was concentrated under reduced pressure. The residue was combined with another batch at 40 mg scale and purified twice by reverse phase HPLC to afford 8-(2,2′-dichloro-3′-(6-methoxy-5-(((((S)-5-oxopyrrolidin-2-yl)methyl)amino)methyl)pyrazin-2-yl)-[1,1′-biphenyl]-3-yl)-3-(((((S)-5-oxopyrrolidin-2-yl)methyl)amino)methyl)-4H-quinolizin-4-one as formic acid salt as a yellow solid. (8.5 mg). m/z found: 726 [M+H]⁺. ¹H NMR (400 MHz, Methanol-d₄): 9.19 (d, J=7.6 Hz, 1H), 8.46 (m, 2H), 7.97-7.92 (m, 2H), 7.67 (dd, J=7.6 Hz, J=38.4 Hz, 1H), 7.61-7.47 (m, 5H), 7.08 (d, J=7.6 Hz, 1H), 4.81 (m, 2H), 4.22-4.13 (m, 2H), 4.10 (s, 3H), 3.99-3.91 (m, 2H), 3.25 (m, 1H), 3.02-2.83 (m, 3H), 2.42-2.29 (m, 6H), 1.90-1.85 (m, 2H).

### Example 108: 8-(2,2′-Dichloro-3′-(6-(methylthio)-5-(((((S)-5-oxopyrrolidin-2-yl)methyl)amino)methyl)pyridin-2-yl)-[1,1′-biphenyl]-3-yl)-3-(((((S)-5-oxopyrrolidin-2-yl)methyl)amino)methyl)-4H-pyrido[1,2-a]pyrimidin-4-one

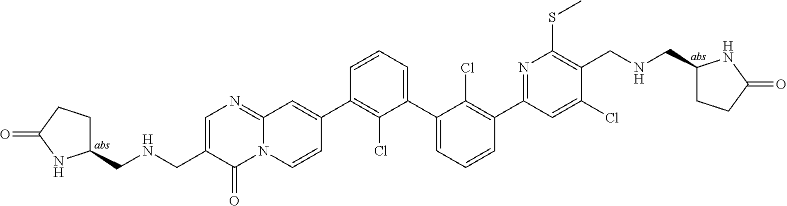 [SMILES: CSc1nc(-c2cccc(-c3cccc(-c4ccn5c(=O)c(CNC[C@@H]6CCC(=O)N6)cnc5c4)c3Cl)c2Cl)cc(Cl)c1CNC[C@@H]1CCC(=O)N1]

### 6-Bromo-2-methylsulfanyl-pyridine-3-carbaldehyde

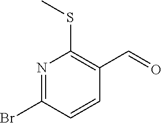

To a solution of 6-bromo-2-fluoro-pyridine-3-carbaldehyde (150 mg, 0.74 mmol) in DMF was added sodium thiomethoxide (51.5 mg, 0.74 mmol) and the mixture was stirred in an ice bath for 2 hours. Water was added and the mixture was extracted into DCM. The organic layer was dried and concentrated. The purification on silica gel column (EtOAc in hexanes, 0 to 20%, as eluent) provided the product. ¹H NMR (400 MHz, Chloroform-d): δ 10.18 (s, 1H), 7.81 (d, J=7.9 Hz, 1H), 7.32 (d, J=7.9 Hz, 1H), 2.61 (s, 3H).

### 8-[2-Chloro-3-[2-chloro-3-(5-formyl-6-methylsulfanyl-2-pyridyl)phenyl]phenyl]-4-oxo-pyrido[1,2-a]pyrimidine-3-carbaldehyde

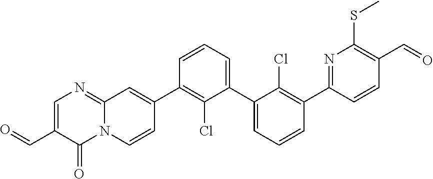 [SMILES: CSc1nc(-c2cccc(-c3cccc(-c4ccn5c(=O)c(C=O)cnc5c4)c3Cl)c2Cl)ccc1C=O]

To a solution of [2-chloro-3-[2-chloro-3-(3-formyl-4-oxo-pyrido[1,2-a]pyrimidin-8-yl)phenyl]phenyl]boronic acid (18 mg, 0.04 mmol), 6-bromo-2-methylsulfanyl-pyridine-3-carbaldehyde (12.37 mg, 0.05 mmol) and potassium carbonate (17 mg, 0.12 mmol) in degassed dioxane/water (6:1) was added Pd(PPh₃)₄ (5 mg, 0.00 mmol) and the mixture was stirred at 90° C. for 4 hours under N₂(g). Water was added and the mixture was extracted into EtOAc three times. The combined organic layers were dried over anhydrous sodium sulfate, concentrated, and purified on silica gel column using EtOAc in hexanes (0 to 60% gradient). Yield: 17 mg (76%). MS: m/z found 546.1 [M+H]⁺.

### 8-[2-Chloro-3-[2-chloro-3-[6-methylsulfanyl-5-[[[(2S)-5-oxopyrrolidin-2-yl]methylamino]methyl]-2-pyridyl]phenyl]phenyl]-3-[[[(2S)-5-oxopyrrolidin-2-yl]methylamino]methyl]pyrido[1,2-a]pyrimidin-4-one

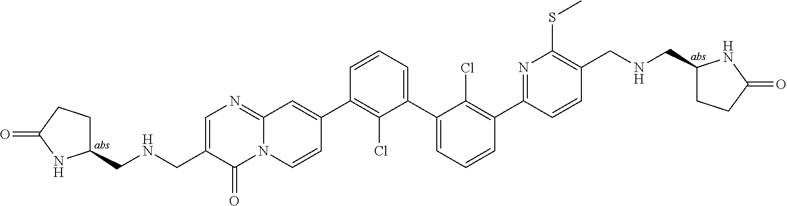 [SMILES: CSc1nc(-c2cccc(-c3cccc(-c4ccn5c(=O)c(CNC[C@@H]6CCC(=O)N6)cnc5c4)c3Cl)c2Cl)ccc1CNC[C@@H]1CCC(=O)N1]

A mixture of 8-[2-chloro-3-[2-chloro-3-(5-formyl-6-methylsulfanyl-2-pyridyl)phenyl]phenyl]-4-oxo-pyrido[1,2-a]pyrimidine-3-carbaldehyde (40 mg, 0.07 mmol), (5S)-5-(aminomethyl)pyrrolidin-2-one (33.42 mg, 0.29 mmol) and acetic acid (8 uL, 9 mg, 0.15 mmol) in MeOH/THF (1:1) was stirred for 1.5 hour at rt. NaBH₃CN (18 mg, 0.29 mmol) was added, the mixture was stirred for additional 5 h, quenched with water and directly purified by HPLC. The collected fraction was neutralized with sat. NaHCO₃(aq.), acetonitrile was evaporated, and aqueous phase was extracted into DCM three times. The combined organic extracts were dried over anhydrous magnesium sulfate and concentrated in vacuo to give 8-[2-chloro-3-[2-chloro-3-[6-methylsulfanyl-5-[[[(2S)-5-oxopyrrolidin-2-yl]methylamino]methyl]-2-pyridyl]phenyl]phenyl]-3-[[[(2S)-5-oxopyrrolidin-2-yl]methylamino]methyl]pyrido[1,2-a]pyrimidin-4-one (45 mg, 82.8%). MS: m/z found 742.2 [M+H]⁺; ¹H NMR (400 MHz, Methanol-d₄): δ9.12 (dt, J=7.4, 1.0 Hz, 1H), 8.41 (d, J=1.1 Hz, 1H), 7.77-7.71 (m, 2H), 7.69-7.65 (m, 1H), 7.64-7.56 (m, 2H), 7.54-7.48 (m, 3H), 7.41-7.37 (m, 2H), 3.92-3.77 (m, 6H), 2.78-2.66 (m, 4H), 2.60 (s, 3H), 2.37-2.24 (m, 6H), 1.89-1.77 (m, 2H).

### Example 109: 8-(2,2′-Dichloro-3′-(6-cyclopropoxy-5-(((((S)-5-oxopyrrolidin-2-yl)methyl)amino)methyl)pyridin-2-yl)-[1,1′-biphenyl]-3-yl)-3-(((((S)-5-oxopyrrolidin-2-yl)methyl)amino)methyl)-4H-pyrido[1,2-a]pyrimidin-4-one

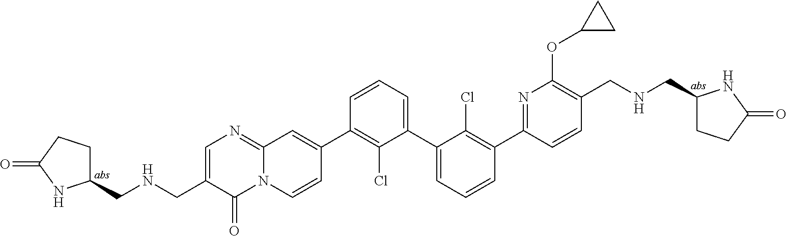 [SMILES: O=C1CC[C@@H](CNCc2ccc(-c3cccc(-c4cccc(-c5ccn6c(=O)c(CNC[C@@H]7CCC(=O)N7)cnc6c5)c4Cl)c3Cl)nc2OC2CC2)N1]

### 6-Bromo-2-(cyclopropoxy)pyridine-3-carbaldehyde

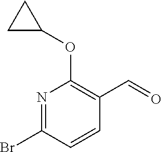 [SMILES: O=Cc1ccc(Br)nc1OC1CC1]

To a solution of 6-bromo-2-fluoro-pyridine-3-carbaldehyde (100 mg, 0.49 mmol) in DMF was added cyclopropanol (3 equivalents) and K₂CO₃ (5 equivalents) and the mixture was stirred at 80° C. for 12 hours. After cooling down to rt, water was added, and the mixture was extracted into ethyl acetate. The organic layer was dried over anhydrous sodium sulfate, filtered, and concentrated. The purification on silica gel column using EtOAc in hexanes (10%) afforded 6-bromo-2-(cyclopropoxy)pyridine-3-carbaldehyde (45 mg, 37.9%). MS: m/z found 242.0 [M+H]⁺; ¹H NMR (400 MHz, Chloroform-d): δ10.21 (d, J=0.7 Hz, 1H), 7.92 (dd, J=7.8, 0.5 Hz, 1H), 7.22 (dt, J=7.9, 0.6 Hz, 1H), 4.55-4.46 (m, 1H), 0.90-0.83 (m, 4H).

### 8-[2-Chloro-3-[2-chloro-3-[6-(cyclopropoxy)-5-formyl-2-pyridyl]phenyl]phenyl]-4-oxo-pyrido[1,2-a]pyrimidine-3-carbaldehyde

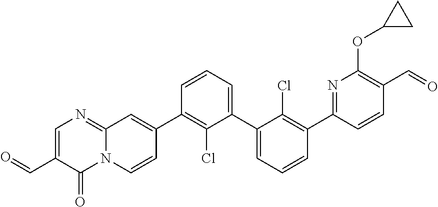 [SMILES: O=Cc1ccc(-c2cccc(-c3cccc(-c4ccn5c(=O)c(C=O)cnc5c4)c3Cl)c2Cl)nc1OC1CC1]

To a solution of 8-[2-chloro-3-[2-chloro-3-(4,4,5,5-tetramethyl-1,3,2-dioxaborolan-2-yl)phenyl]phenyl]-4-oxo-pyrido[1,2-a]pyrimidine-3-carbaldehyde (40 mg, 0.08 mmol), 6-bromo-2-(cyclopropoxy)pyridine-3-carbaldehyde (20 mg, 0.08 mmol) and potassium carbonate (32 mg, 0.23 mmol) in degassed dioxane/water (6:1) was added Pd(PPh₃)₄ (9 mg, 0.01 mmol) and the mixture was stirred at 90° C. for 4 hours under N₂(g). Water was added and the mixture was extracted into EtOAc three times. The combined organic layers were dried over anhydrous sodium sulfate, concentrated, and purified on silica gel column using EtOAc in hexanes (0 to 60% gradient) to provide 8-[2-chloro-3-[2-chloro-3-[6-(cyclopropoxy)-5-formyl-2-pyridyl]phenyl]phenyl]-4-oxo-pyrido[1,2-a]pyrimidine-3-carbaldehyde (30 mg, 70.3%). MS: m/z found 556.1 [M+H]⁺.

### 8-[2-Chloro-3-[2-chloro-3-[6-(cyclopropoxy)-5-[[[(2S)-5-oxopyrrolidin-2-yl]methylamino]methyl]-2-pyridyl]phenyl]phenyl]-3-[[[(2S)-5-oxopyrrolidin-2-yl]methylamino]methyl]pyrido[1,2-a]pyrimidin-4-one

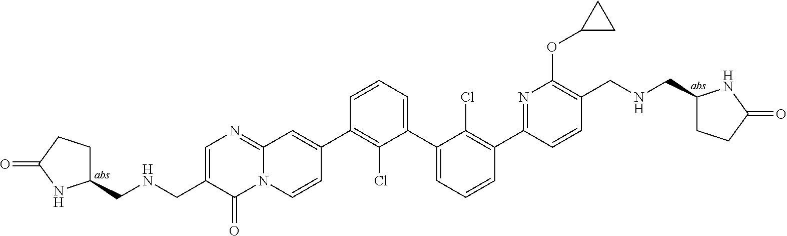 [SMILES: O=C1CC[C@@H](CNCc2ccc(-c3cccc(-c4cccc(-c5ccn6c(=O)c(CNC[C@@H]7CCC(=O)N7)cnc6c5)c4Cl)c3Cl)nc2OC2CC2)N1]

A mixture of 8-[2-chloro-3-[2-chloro-3-[6-(cyclopropoxy)-5-formyl-2-pyridyl]phenyl]phenyl]-4-oxo-pyrido[1,2-a]pyrimidine-3-carbaldehyde (31.00 mg, 0.06 mmol), (5S)-5-(aminomethyl)pyrrolidin-2-one (25 mg, 0.22 mmol) and acetic acid (6 uL, 7 mg, 0.11 mmol) in MeOH/THF (1:1) was stirred for 1.5 hour at rt. NaBH₃CN (14 mg, 0.22 mmol) was added, the mixture was stirred for additional 5 h, quenched with water and directly purified by HPLC. The collected fraction was neutralized with sat. NaHCO₃(aq.), acetonitrile was evaporated, and aqueous phase was extracted into DCM three times. The combined organic extracts were dried over anhydrous magnesium sulfate and concentrated in vacuo to give 8-[2-chloro-3-[2-chloro-3-[6-(cyclopropoxy)-5-[[[(2S)-5-oxopyrrolidin-2-yl]methylamino]methyl]-2-pyridyl]phenyl]phenyl]-3-[[[(2S)-5-oxopyrrolidin-2-yl]methylamino]methyl]pyrido[1,2-a]pyrimidin-4-one (30 mg, 71.5%). MS: m/z found 752.3 [M+H]⁺; ¹H NMR (400 MHz, Methanol-d₄) δ 9.14 (dt, J=7.4, 1.0 Hz, 1H), 8.42 (d, J=1.0 Hz, 1H), 7.78-7.73 (m, 2H), 7.68 (ddd, J=7.7, 1.8, 1.0 Hz, 1H), 7.65-7.58 (m, 2H), 7.56-7.50 (m, 3H), 7.41 (ddd, J=7.6, 1.8, 1.0 Hz, 1H), 7.29 (dd, J=7.5, 1.0 Hz, 1H), 4.42 (tt, J=6.6, 3.4 Hz, 1H), 3.92-3.80 (m, 4H), 3.77 (d, J=3.5 Hz, 2H), 2.79-2.62 (m, 4H), 2.37-2.24 (m, 6H), 1.87-1.76 (m, 2H), 0.81-0.74 (m, 4H).

### Example 110: 8-(2,2′-Dichloro-3″-(difluoromethoxy)-4″-(((((S)-5-oxopyrrolidin-2-yl)methyl)amino)methyl)-[1,1′:3′,1″-terphenyl]-3-yl)-3-(((((S)-5-oxopyrrolidin-2-yl)methyl)amino)methyl)-4H-pyrido[1,2-a]pyrimidin-4-one

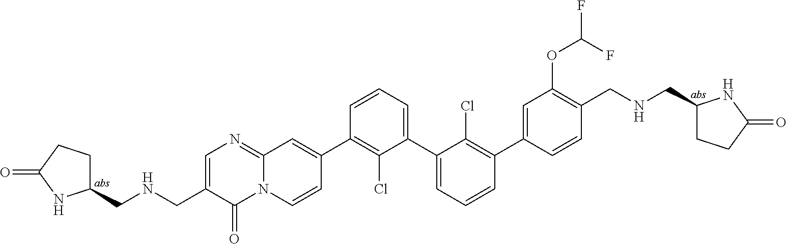 [SMILES: O=C1CC[C@@H](CNCc2ccc(-c3cccc(-c4cccc(-c5ccn6c(=O)c(CNC[C@@H]7CCC(=O)N7)cnc6c5)c4Cl)c3Cl)cc2OC(F)F)N1]

### 8-12-Chloro-3-12-chloro-3-[3-(difluoromethoxy)-4-formyl-phenyl]phenyllphenyl]-4-oxo-pyrido[1,2-a]pyrimidine-3-carbaldehyde

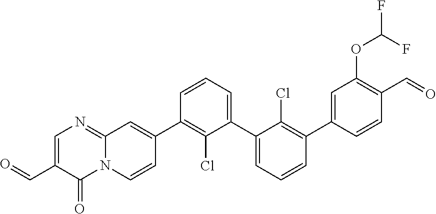 [SMILES: O=Cc1ccc(-c2cccc(-c3cccc(-c4ccn5c(=O)c(C=O)cnc5c4)c3Cl)c2Cl)cc1OC(F)F]

To a solution of 8-[2-chloro-3-[2-chloro-3-(4,4,5,5-tetramethyl-1,3,2-dioxaborolan-2-yl)phenyl]phenyl]-4-oxo-pyrido[1,2-a]pyrimidine-3-carbaldehyde (50 mg, 0.10 mmol), 4-bromo-2-(difluoromethoxy)benzaldehyde (31 mg, 0.12 mmol) and potassium carbonate (40 mg, 0.29 mmol) in degassed dioxane/water (6:1) was added Pd(PPh₃)₄ (11 mg, 0.01 mmol) and the mixture was stirred at 90° C. for 4 hours under N₂(g). Water was added and the mixture was extracted into EtOAc three times. The combined organic layers were dried over anhydrous sodium sulfate, concentrated, and purified on silica gel column using EtOAc in hexanes (0 to 60% gradient) to afford 8-[2-chloro-3-[2-chloro-3-[3-(difluoromethoxy)-4-formyl-phenyl]phenyl]phenyl]-4-oxo-pyrido[1,2-a]pyrimidine-3-carbaldehyde (22 mg, 40.6%). MS: m/z found 565.1 [M+H]⁺.

### 8-[2-Chloro-3-[2-chloro-3-[3-(difluoromethoxy)-4-[[[(2S)-5-oxopyrrolidin-2-yl]methylamino]methyl]phenyl]phenyl]phenyl]-3-[[[(2S)-5-oxopyrrolidin-2-yl]methylamino]methyl]pyrido[1,2-a]pyrimidin-4-one

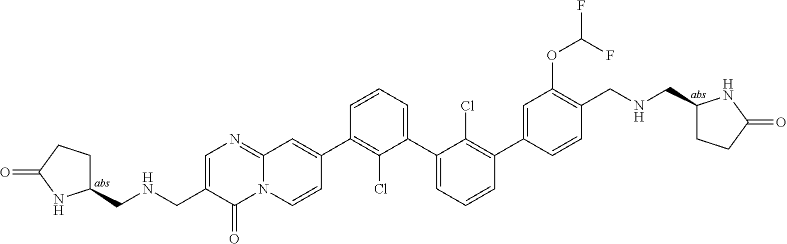 [SMILES: O=C1CC[C@@H](CNCc2ccc(-c3cccc(-c4cccc(-c5ccn6c(=O)c(CNC[C@@H]7CCC(=O)N7)cnc6c5)c4Cl)c3Cl)cc2OC(F)F)N1]

A mixture of 8-[2-chloro-3-[2-chloro-3-[3-(difluoromethoxy)-4-formyl-phenyl]phenyl]phenyl]-4-oxo-pyrido[1,2-a]pyrimidine-3-carbaldehyde (37. mg, 0.07 mmol), (5S)-5-(aminomethyl)pyrrolidin-2-one (30 mg, 0.26 mmol) and acetic acid (7.5 uL, 8 mg, 0.13 mmol) in MeOH/THF (1:1) was stirred for 1.5 hour at rt. NaBH₃CN (16 mg, 0.26 mmol) was added, the mixture was stirred for additional 5 h, quenched with water and directly purified by HPLC. The collected fraction was neutralized with sat. NaHCO₃(aq.), acetonitrile was evaporated, and aqueous phase was extracted into DCM three times. The combined organic extracts were dried over anhydrous magnesium sulfate and concentrated in vacuo to give 8-[2-chloro-3-[2-chloro-3-[3-(difluoromethoxy)-4-[[[(2S)-5-oxopyrrolidin-2-yl]methylamino]methyl]phenyl]phenyl]phenyl]-3-[[[(2S)-5-oxopyrrolidin-2-yl]methylamino]methyl]pyrido[1,2-a]pyrimidin-4-one (28 mg, 56.2%). MS: m/z found 761.2 [M+H]⁺; ¹H NMR (400 MHz, Methanol-d₄): δ 9.13 (dd, J=7.4, 0.8 Hz, 1H), 8.41 (s, 1H), 7.76 (dd, J=1.9, 0.7 Hz, 1H), 7.64-7.59 (m, 2H), 7.57-7.45 (m, 5H), 7.38 (dd, J=7.2, 2.0 Hz, 1H), 7.33 (dd, J=7.9, 1.7 Hz, 1H), 7.27 (d, J=1.4 Hz, 1H), 6.93 (td, J=74.0, 1.2 Hz, 1H), 3.90 (d, J=1.6 Hz, 2H), 3.88-3.81 (m, 4H), 2.77-2.66 (m, 4H), 2.36-2.24 (m, 6H), 1.87-1.77 (m, 2H).

### Example 111: (S)-8-(2,2′-Dichloro-3′-(6-methoxy-5-((((5-oxopyrrolidin-2-yl)methyl)amino)methyl)pyridin-2-yl)-[1,1′-biphenyl]-3-yl)-3-((3-(2-hydroxypropan-2-yl)azetidin-1-yl)methyl)-4H-pyrido[1,2-a]pyrimidin-4-one

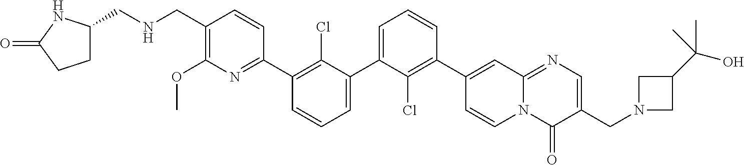 [SMILES: COc1nc(-c2cccc(-c3cccc(-c4ccn5c(=O)c(CN6CC(C(C)(C)O)C6)cnc5c4)c3Cl)c2Cl)ccc1CNC[C@@H]1CCC(=O)N1]

tert-Butyl N-[[6-[2-chloro-3-[2-chloro-3-(3-formyl-4-oxo-pyrido[1,2-a]pyrimidin-8-yl)phenyl]phenyl]-2-methoxy-3-pyridyl]methyl]-N-[[(2S)-5-oxopyrrolidin-2-yl]methyl]carbamate (25 mg, 0.03 mmol), 2-(azetidin-3-yl)propan-2-ol (12 mg, 0.10 mmol) and acetic acid (4 mg, 0.07 mmol) were dissolved in 1 ml of 1:1 MeOH/THF solvent mix, and the reaction was stirred at 25° C. for 3 hours. NaBH₃CN (4 mg, 0.07 mmol) was added portion-wise, and the resulting mixture was stirred at 25° C. for 1 hour. The reaction was concentrated and the residue was dissolved in water (3 ml), then extracted with DCM (3×2 ml). The combined organics were further washed with brine, dried over sodium sulfate, then filtered and concentrated. Crude material was purified by silica column, eluting with 0-10% MeOH gradient in DCM to afford tert-butyl N-[[6-[2-chloro-3-[2-chloro-3-[3-[[3-(1-hydroxy-1-methyl-ethyl)azetidin-1-yl]methyl]-4-oxo-pyrido[1,2-a]pyrimidin-8-yl]phenyl]phenyl]-2-methoxy-3-pyridyl]methyl]-N-[[(2S)-5-oxopyrrolidin-2-yl]methyl]carbamate (18.6 mg, 65.5% yield) as a yellow foam. MS: m/z found 827.45, 829 [M+H]⁺.

tert-butylN-[[6-[2-chloro-3-[2-chloro-3-[3-[[3-(1-hydroxy-1-methyl-ethyl)azetidin-1-yl]methyl]-4-oxo-pyrido[1,2-a]pyrimidin-8-yl]phenyl]phenyl]-2-methoxy-3-pyridyl]methyl]-N-[[(2S)-5-oxopyrrolidin-2-yl]methyl]carbamate (18.5 mg, 0.02 mmol) was dissolved in 0.5 ml DCM and 4M HCl solution in dioxane (28 μL, 4 mg, 0.11 mmol) was added. The reaction was stirred at rt for 20 minutes and the mixture was subsequently concentrated. Crude sample was purified by reverse phase HPLC to afford 8-[2-chloro-3-[2-chloro-3-[6-methoxy-5-[[[(2S)-5-oxopyrrolidin-2-yl]methylamino]methyl]-2-pyridyl]phenyl]phenyl]-3-[[3-(1-hydroxy-1-methyl-ethyl)azetidin-1-yl]methyl]pyrido[1,2-a]pyrimidin-4-one (2.4 mg, 14.8% yield) as a clear film. MS: m/z found 727.35, 729.35 [M+H]⁺. ¹H NMR (400 MHz, Chloroform-d) δ 9.08 (d, J=7.4 Hz, 1H), 8.42 (s, 1H), 7.76 (s, 1H), 7.69 (dd, J=7.6, 1.7 Hz, 1H), 7.59 (d, J=7.5 Hz, 1H), 7.53-7.42 (m, 4H), 7.40-7.31 (m, 2H), 7.28 (s, 1H), 5.93 (s, 1H), 4.03 (s, 3H), 3.81 (s, 5H), 3.64-3.40 (m, 3H), 2.80 (dd, J=12.0, 4.1 Hz, 1H), 2.56 (dd, J=12.0, 8.5 Hz, 2H), 2.37 (t, J=8.0 Hz, 2H), 2.25 (dt, J=12.8, 7.4 Hz, 1H), 1.83-1.73 (m, 2H), 1.17 (s, 6H), 0.86 (d, J=16.9 Hz, 1H).

### Example 112: (S)-8-(2,2′-Dichloro-3′-(5-((3-(2-hydroxypropan-2-yl)azetidin-1-yl)methyl)-6-methoxypyridin-2-yl)-[1,1′-biphenyl]-3-yl)-3-((((5-oxopyrrolidin-2-yl)methyl)amino)methyl)-4H-pyrido[1,2-a]pyrimidin-4-one

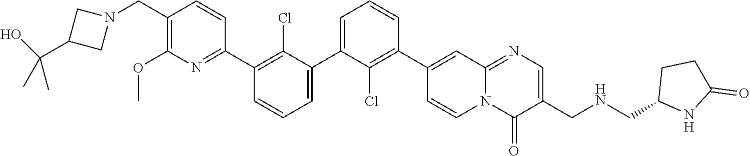 [SMILES: COc1nc(-c2cccc(-c3cccc(-c4ccn5c(=O)c(CNC[C@@H]6CCC(=O)N6)cnc5c4)c3Cl)c2Cl)ccc1CN1CC(C(C)(C)O)C1]

tert-Butyl N-[[8-[2-chloro-3-[2-chloro-3-(5-formyl-6-methoxy-2-pyridyl)phenyl]phenyl]-4-oxo-pyrido[1,2-a]pyrimidin-3-yl]methyl]-N-[[(2S)-5-oxopyrrolidin-2-yl]methyl]carbamate (26 mg, 0.04 mmol), 2-(azetidin-3-yl)propan-2-ol (12 mg, 0.11 mmol) and acetic acid (4.mg, 0.07 mmol) were dissolved in 1 ml of 1:1 MeOH/THF solvent mix, and the reaction was stirred at 25° C. for 3 hours. NaBH₃CN (4.5 mg, 0.07 mmol) was added portion-wise, and the resulting mixture was stirred at 25° C. for 1 hour. The reaction was concentrated and the residue was dissolved in water (3 mL), then extracted with DCM (3×2 mL). The combined organics were further washed with brine, dried over sodium sulfate, then filtered and concentrated to afford tert-butyl N-[[8-[2-chloro-3-[2-chloro-3-[5-[[3-(1-hydroxy-1-methyl-ethyl)azetidin-1-yl]methyl]-6-methoxy-2-pyridyl]phenyl]phenyl]-4-oxo-pyrido[1,2-a]pyrimidin-3-yl]methyl]-N-[[(2S)-5-oxopyrrolidin-2-yl]methyl]carbamate (29 mg) as a yellow oil. Material was used as—is for the next step. MS: m/z found 827, 829 [M+H]⁺. tert-butyl N-[[8-[2-chloro-3-[2-chloro-3-[5-[[3-(1-hydroxy-1-methyl-ethyl)azetidin-1-yl]methyl]-6-methoxy-2-pyridyl]phenyl]phenyl]-4-oxo-pyrido[1,2-a]pyrimidin-3-yl]methyl]-N-[[(2S)-5-oxopyrrolidin-2-yl]methyl]carbamate (29.00 mg, 0.04 mmol) was dissolved in 0.5 ml DCM and 4M HCl solution in dioxane (45 μL, 6.5 mg, 0.18 mmol) was added. The reaction was stirred at rt for 20 minutes and the mixture was subsequently concentrated. Crude sample was purified by reverse phase HPLC to afford 8-[2-chloro-3-[2-chloro-3-[5-[[3-(1-hydroxy-1-methyl-ethyl)azetidin-1-yl]methyl]-6-methoxy-2-pyridyl]phenyl]phenyl]-3-[[[(2S)-5-oxopyrrolidin-2-yl]methylamino]methyl]pyrido[1,2-a]pyrimidin-4-one (12.7 mg, 48.2% yield) as a light yellow foam. MS: m/z found 727.3, 729.3 [M+H]⁺. ¹H NMR (400 MHz, Chloroform-d) δ 9.07 (d, J=7.3 Hz, 1H), 8.38 (s, 1H), 7.98 (d, J=7.6 Hz, 1H), 7.75 (s, 1H), 7.66 (d, J=7.4 Hz, 1H), 7.47 (dq, J=9.6, 7.6 Hz, 4H), 7.38 (dd, J=7.6, 1.9 Hz, 1H), 7.33 (dd, J=7.5, 5.7 Hz, 2H), 6.47 (s, 1H), 4.17 (s, 2H), 4.05 (s, 4H), 3.98-3.81 (m, 5H), 2.86 (dd, J=11.2, 3.8 Hz, 2H), 2.64 (t, J=10.6 Hz, 1H), 2.39-2.17 (m, 3H), 1.81-1.66 (m, 1H), 1.16 (s, 6H).

### Example 113: Methyl ((6-(2,2′-dichloro-3′-(4-oxo-3-(((((S)-5-oxopyrrolidin-2-yl)methyl)amino)methyl)-4H-pyrido[1,2-a]pyrimidin-8-yl)-[1,1′-biphenyl]-3-yl)-2-methoxypyridin-3-yl)methyl)-L-serinate

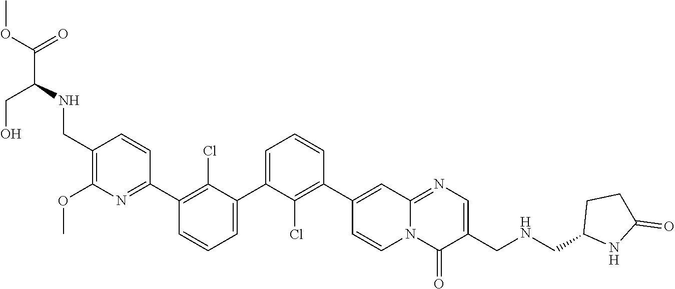 [SMILES: COC(=O)[C@H](CO)NCc1ccc(-c2cccc(-c3cccc(-c4ccn5c(=O)c(CNC[C@@H]6CCC(=O)N6)cnc5c4)c3Cl)c2Cl)nc1OC]

tert-butyl N-[[8-[2-chloro-3-[2-chloro-3-(5-formyl-6-methoxy-2-pyridyl)phenyl]phenyl]-4-oxo-pyrido[1,2-a]pyrimidin-3-yl]methyl]-N-[[(2S)-5-oxopyrrolidin-2-yl]methyl]carbamate (50 mg, 0.07 mmol), methyl (2S)-2-amino-3-hydroxy-propanoate;hydrochloride (32 mg, 0.21 mmol) and DIEA (36 μL, 0.21 mmol) were dissolved in 1 ml of 1:1 MeOH/THF solvent mix, and the reaction was stirred at 25° C. for 3 hours. NaBH₃CN (9 mg, 0.14 mmol), along with acetic acid, (8 mg, 0.13 mmol) were added, and the resulting mixture was stirred at 25° C. for 1 hour. Reaction mixture was diluted with water (2 ml), then extracted with DCM (3×2 mL). The combined organics were further washed with brine, dried over sodium sulfate, then filtered and concentrated. The crude mixture was purified by silica gel chromatography, eluting with 0-10% MeOH gradient in DCM to afford methyl (2S)-2-[[6-[3-[3-[3-[[tert-butoxycarbonyl-[[(2S)-5-oxopyrrolidin-2-yl]methyl]amino]methyl]-4-oxo-pyrido[1,2-a]pyrimidin-8-yl]-2-chloro-phenyl]-2-chloro-phenyl]-2-methoxy-3-pyridyl]methyl amino]-3-hydroxy-propanoate (57 mg, 99.9% yield) as an orange oil. MS: m/z found 831, 833 [M+H]⁺. methyl (2S)-2-[[6-[3-[3-[3-[[tert-butoxycarbonyl-[[(2S)-5-oxopyrrolidin-2-yl]methyl]amino]methyl]-4-oxo-pyrido[1,2-a] pyrimidin-8-yl]-2-chloro-phenyl]-2-chloro-phenyl]-2-methoxy-3-pyridyl]methyl amino]-3-hydroxy-propanoate (57 mg, 0.07 mmol) was dissolved in 0.5 ml DCM and 4M HCl solution in dioxane (86 μL, 12.34 mg, 0.34 mmol) was added. The reaction was stirred at rt for 20 minutes and the mixture was subsequently concentrated. Crude sample was purified by reverse phase HPLC to afford methyl (2S)-2-[[6-[2-chloro-3-[2-chloro-3-[4-oxo-3-[[[(2S)-5-oxopyrrolidin-2-yl]methylamino] methyl]pyrido[1,2-a]pyrimidin-8-yl]phenyl]phenyl]-2-methoxy-3-pyridyl]methyl amino]-3-hydroxy-propanoate (13 mg, 25.9% yield) as a clear film. MS: m/z found 731, 733 [M+H]⁺. ¹H NMR (400 MHz, Chloroform-d) δ 9.07 (d, J=7.2 Hz, 1H), 8.37 (s, 1H), 7.76 (s, 1H), 7.68 (dd, J=7.6, 1.9 Hz, 1H), 7.61 (d, J=7.4 Hz, 1H), 7.53-7.41 (m, 4H), 7.39 (d, J=7.5 Hz, 1H), 7.33 (dd, J=7.4, 1.9 Hz, 1H), 7.26 (d, J=3.1 Hz, 1H), 6.43 (s, 1H), 4.03 (s, 3H), 3.91 (d, J=17.3 Hz, 2H), 3.84 (dd, J=6.9, 4.3 Hz, 4H), 3.75-3.61 (m, 4H), 3.48 (t, J=5.4 Hz, 1H), 2.88 (dd, J=12.0, 4.0 Hz, 1H), 2.66 (d, J=10.5 Hz, 1H), 2.35 (t, J=8.5 Hz, 2H), 2.25 (dq, J=15.4, 8.0, 7.3 Hz, 1H), 1.77 (t, J=9.8 Hz, 1H), 0.86 (d, J=11.7 Hz, 1H).

### Example 114: ((6-(2,2′-Dichloro-3′-(4-oxo-3-(((((S)-5-oxopyrrolidin-2-yl)methyl)amino)methyl)-4H-pyrido[1,2-a]pyrimidin-8-yl)-[1,1′-biphenyl]-3-yl)-2-methoxypyridin-3-yl)methyl)-L-serine

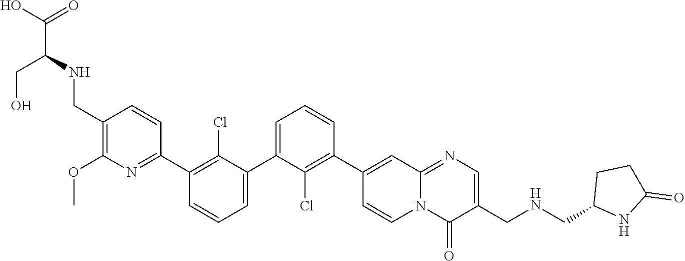 [SMILES: COc1nc(-c2cccc(-c3cccc(-c4ccn5c(=O)c(CNC[C@@H]6CCC(=O)N6)cnc5c4)c3Cl)c2Cl)ccc1CN[C@@H](CO)C(=O)O]

Methyl (2S)-2-[[6-[2-chloro-3-[2-chloro-3-[4-oxo-3-[[[(2S)-5-oxopyrrolidin-2-yl]methylamino]methyl]pyrido[1,2-a]pyrimidin-8-yl]phenyl]phenyl]-2-methoxy-3-pyridyl]methylamino]-3-hydroxy-propanoate (10.5 mg, 0.01 mmol) was dissolved in 0.5 ml THE and hydroxylithium;hydrate (2 mg, 0.04 mmol) in 0.1 mL water was added. The resulting mixture was stirred at rt for 1 hour. The reaction was concentrated and the crude sample was purified by reverse phase HPLC to afford (2S)-2-[[6-[2-chloro-3-[2-chloro-3-[4-oxo-3-[[[(2S)-5-oxopyrrolidin-2-yl]methylamino]methyl]pyrido[1,2-a]pyrimidin-8-yl]phenyl]phenyl]-2-methoxy-3-pyridyl]methylamino]-3-hydroxy-propanoic acid (6.7 mg, 65.1% yield) as a white fluffy solid (bis formic acid salt). MS: m/z found 717.35, 719 [M+H]⁺. ¹H NMR (400 MHz, Methanol-d₄) δ 8.47 (s, 1H), 8.41 (s, 2H), 7.87 (d, J=7.6 Hz, 1H), 7.82 (s, 1H), 7.67 (dd, J=7.6, 1.7 Hz, 1H), 7.65-7.56 (m, 3H), 7.53 (t, J=7.6 Hz, 2H), 7.42 (dd, J=7.6, 1.7 Hz, 1H), 7.33 (d, J=7.5 Hz, 1H), 4.37 (s, 2H), 4.11 (d, J=3.9 Hz, 2H), 4.09 (s, 3H), 4.05 (dd, J=11.9, 3.8 Hz, 1H), 3.97 (s, 1H), 3.89 (dd, J=11.9, 7.0 Hz, 1H), 3.63 (dd, J=6.9, 3.8 Hz, 1H), 3.00 (qd, J=12.6, 6.2 Hz, 2H), 2.41-2.31 (m, 3H), 1.87 (q, J=4.9, 4.3 Hz, 1H).

### Example 115: (S)-1-(((6-(2,2′-Dichloro-3′-(4-oxo-3-((((5-oxopyrrolidin-2-yl)methyl)amino)methyl)-4H-pyrido[1,2-a]pyrimidin-8-yl)-[1,1′-biphenyl]-3-yl)-2-methoxypyridin-3-yl)methyl)amino)cyclopropane-1-carboxylic acid

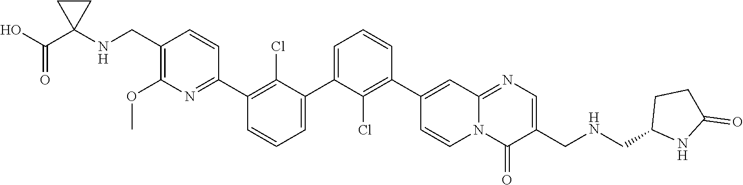 [SMILES: COc1nc(-c2cccc(-c3cccc(-c4ccn5c(=O)c(CNC[C@@H]6CCC(=O)N6)cnc5c4)c3Cl)c2Cl)ccc1CNC1(C(=O)O)CC1]

tert-butyl N-[[8-[2-chloro-3-[2-chloro-3-(5-formyl-6-methoxy-2-pyridyl)phenyl]phenyl]-4-oxo-pyrido[1,2-a]pyrimidin-3-yl]methyl]-N-[[(2S)-5-oxopyrrolidin-2-yl]methyl]carbamate (35 mg, 0.05 mmol), 1-aminocyclopropanecarboxylic acid (15 mg, 0.14 mmol) and acetic acid (6 mg, 0.10 mmol) were dissolved in 1 ml of 1:1 MeOH/THF solvent mix, and the reaction was stirred at 25° C. for 3 hours. NaBH₃CN (6 mg, 0.10 mmol) was then added portion-wise, and the resulting mixture was stirred at 25° C. for 1 hour. The reaction was concentrated and the residue was dissolved in 1 ml of 1M aqueous HCl solution. The aqueous was extracted with DCM (2×1 ml). The combined organics were washed with 2 mL brine, dried over sodium sulfate, then filtered and concentrated to afford 1-[[6-[3-[3-[3-[[tert-butoxycarbonyl-[[(2S)-5-oxopyrrolidin-2-yl]methyl]amino]methyl]-4-oxo-pyrido[1,2-a]pyrimidin-8-yl]-2-chloro-phenyl]-2-chloro-phenyl]-2-methoxy-3-pyridyl]methylamino]cyclopropanecarboxylic acid (39 mg) as a yellow oil. Material was used as—is for the next step. MS: m/z found 813, 815 [M+H]⁺.

1-[[6-[3-[3-[3-[[tert-butoxycarbonyl-[[(2S)-5-oxopyrrolidin-2-yl]methyl]amino]methyl]-4-oxo-pyrido[1,2-a]pyrimidin-8-yl]-2-chloro-phenyl]-2-chloro-phenyl]-2-methoxy-3-pyridyl]methylamino]cyclopropanecarboxylic acid (39.00 mg, 0.05 mmol) was dissolved in 0.5 ml DCM and 4M HCl solution in dioxane (59.91 μL, 8.63 mg, 0.24 mmol) was added. The reaction was stirred at rt for 20 minutes and the mixture was subsequently concentrated. Crude sample was purified by reverse phase HPLC to afford (S)-1-(((6-(2,2′-dichloro-3′-(4-oxo-3-((((5-oxopyrrolidin-2-yl)methyl)amino)methyl)-4H-pyrido[1,2-a]pyrimidin-8-yl)-[1,1′-biphenyl]-3-yl)-2-methoxypyridin-3-yl)methyl)amino)cyclopropane-1-carboxylic acid (8 mg, 23.4% yield) as a white fluffy solid (bis formic acid salt). MS: m/z found 713.55, 715 [M+H]⁺. ¹H NMR (400 MHz, Methanol-d₄) δ 9.16 (d, J=7.4 Hz, 1H), 8.45 (d, J=5.9 Hz, 3H), 7.87 (d, J=7.4 Hz, 1H), 7.82-7.76 (m, 1H), 7.68-7.61 (m, 2H), 7.57 (dd, J=15.7, 8.1 Hz, 2H), 7.51 (d, J=7.7 Hz, 2H), 7.42 (d, J=7.8 Hz, 1H), 7.30 (d, J=7.6 Hz, 1H), 4.39 (s, 2H), 4.05 (d, J=11.3 Hz, 5H), 3.93 (s, 1H), 2.98-2.86 (m, 2H), 2.40-2.29 (m, 3H), 1.85 (s, 1H), 1.39 (d, J=2.3 Hz, 2H), 1.20 (s, 2H).

### Example 116: (S)-1-((8-(2,2′-Dichloro-3′-(6-methoxy-5-(((((S)-5-oxopyrrolidin-2-yl)methyl)amino)methyl)pyrazin-2-yl)-[1,1′-biphenyl]-3-yl)-4-oxo-4H-pyrido[1,2-a]pyrimidin-3-yl)methyl)pyrrolidine-3-carboxylic acid

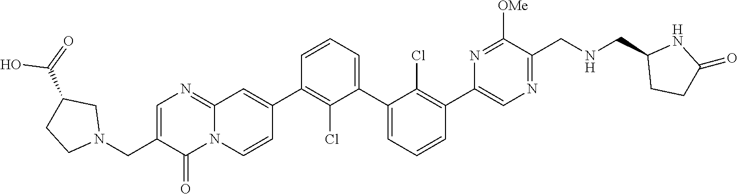 [SMILES: COc1nc(-c2cccc(-c3cccc(-c4ccn5c(=O)c(CN6CC[C@H](C(=O)O)C6)cnc5c4)c3Cl)c2Cl)cnc1CNC[C@@H]1CCC(=O)N1]

### (S)-tert-Butyl ((5-(2,2′-dichloro-3′-(3-formyl-4-oxo-4H-pyrido[1,2-a]pyrimidin-8-yl)-[1,1′-biphenyl]-3-yl)-3-methoxypyrazin-2-yl)methyl)((5-oxopyrrolidin-2-yl)methyl)carbamate

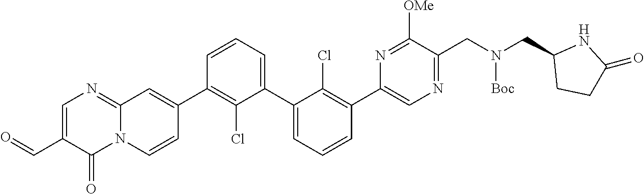 [SMILES: COc1nc(-c2cccc(-c3cccc(-c4ccn5c(=O)c(C=O)cnc5c4)c3Cl)c2Cl)cnc1CN(C[C@@H]1CCC(=O)N1)C(=O)OC(C)(C)C]

To a mixture (S)-tert-butyl ((5-(3-bromo-2-chlorophenyl)-3-methoxypyrazin-2-yl)methyl)((5-oxopyrrolidin-2-yl)methyl)carbamate (0.95 g, 1.81 mmol) in dioxane/H₂O (4:1, 50 mL) was added 8-(2-chloro-3-(4,4,5,5-tetramethyl-1,3,2-dioxaborolan-2-yl)phenyl)-4-oxo-4H-pyrido[1,2-a]pyrimidine-3-carbaldehyde (1.48 g, 3.61 mmol), 1,1′-bis(diphenylphosphino)ferrocene-palladium(ii) dichloride dichloromethane complex (0.012 g, 0.18 mmol) and potassium phosphate (0.96 g, 4.52 mmol). The mixture was stirred at 110° C. for 0.5 hr. To the reaction mixture was added to water (50 mL) then extracted with EtOAc (3×60 ml). The combined organic layers were washed with brine (60 mL), dried over anhydrous sodium sulfate, filtered and concentrated under reduced pressure. The residue was purified by normal phase SiO₂ chromatography (0-100% EtOAc/petroleum ether) to afford (S)-tert-butyl ((5-(2,2′-dichloro-3′-(3-formyl-4-oxo-4H-pyrido[1,2-a]pyrimidin-8-yl)-[1,1′-biphenyl]-3-yl)-3-methoxypyrazin-2-yl)methyl)((5-oxopyrrolidin-2-yl)methyl)carbamate as a yellow solid (1 g, 70% yield). m/z found: 729.2 [M+H]⁺.

### (S)-1-((8-(3′-(5-(((tert-butoxycarbonyl)(((S)-5-oxopyrrolidin-2-yl)methyl)amino)methyl)-6-methoxypyrazin-2-yl)-2,2′-dichloro-[1,1′-biphenyl]-3-yl)-4-oxo-4H-pyrido[1,2-a]pyrimidin-3-yl)methyl)pyrrolidine-3-carboxylic acid

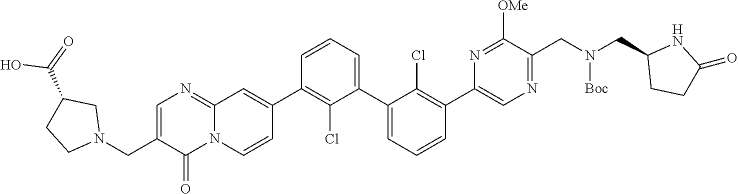 [SMILES: COc1nc(-c2cccc(-c3cccc(-c4ccn5c(=O)c(CN6CC[C@H](C(=O)O)C6)cnc5c4)c3Cl)c2Cl)cnc1CN(C[C@@H]1CCC(=O)N1)C(=O)OC(C)(C)C]

To a mixture (S)-tert-butyl ((5-(2,2′-dichloro-3′-(3-formyl-4-oxo-4H-pyrido[1,2-a]pyrimidin-8-yl)-[1,1′-biphenyl]-3-yl)-3-methoxypyrazin-2-yl)methyl)((5-oxopyrrolidin-2-yl)methyl)carbamate (0.3 g, 411.18 umol, 1 eq) in DCM/MeOH (1:1, 8 mL) was added (S)-pyrrolidine-3-carboxylic acid (0.24 g, 2.06 mmol), sodium acetate (0.1 g, 1.23 mmol). The mixture was stirred at r.t. for 10 hr. Sodium triacetoxyborohydride (0.26 g, 1.23 mmol) was added and the mixture was stirred at r.t. for 2 hr. The reaction mixture was filtered and the filtrate was concentrated under reduced pressure to give (S)-1-((8-(3′-(5-(((tert-butoxycarbonyl)(((S)-5-oxopyrrolidin-2-yl)methyl)amino)methyl)-6-methoxypyrazin-2-yl)-2,2′-dichloro-[1,1′-biphenyl]-3-yl)-4-oxo-4H-pyrido[1,2-a]pyrimidin-3-yl)methyl)pyrrolidine-3-carboxylic acid as a yellow solid (0.3 g, 88% yield). m/z found: 828.3 [M+H]⁺.

### (S)-1-((8-(2,2′-Dichloro-3′-(6-methoxy-5-(((((S)-5-oxopyrrolidin-2-yl)methyl)amino)methyl)pyrazin-2-yl)-[1,1′-biphenyl]-3-yl)-4-oxo-4H-pyrido[1,2-a]pyrimidin-3-yl)methyl)pyrrolidine-3-carboxylic acid

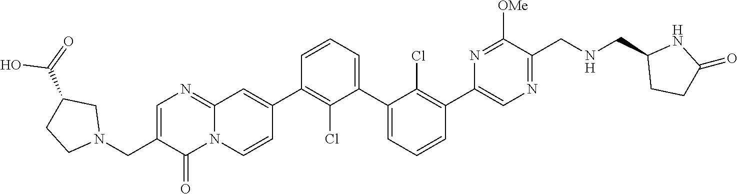 [SMILES: COc1nc(-c2cccc(-c3cccc(-c4ccn5c(=O)c(CN6CC[C@H](C(=O)O)C6)cnc5c4)c3Cl)c2Cl)cnc1CNC[C@@H]1CCC(=O)N1]

To a mixture (S)-1-((8-(3′-(5-(((tert-butoxycarbonyl)(((S)-5-oxopyrrolidin-2-yl)methyl)amino)methyl)-6-methoxypyrazin-2-yl)-2,2′-dichloro-[1,1′-biphenyl]-3-yl)-4-oxo-4H-pyrido[1,2-a]pyrimidin-3-yl)methyl)pyrrolidine-3-carboxylic acid (0.28 g, 0.34 mmol) in DCM (3 mL) was added trifluoroacetic acid (4.99 mL, 67.4 mmol). The mixture was stirred at r.t. for 0.5 hr. The reaction mixture was concentrated under reduced pressure. The residue was purified by reverse phase HPLC to afford (S)-1-((8-(2,2′-dichloro-3′-(6-methoxy-5-(((((S)-5-oxopyrrolidin-2-yl)methyl)amino)methyl)pyrazin-2-yl)-[1,1′-biphenyl]-3-yl)-4-oxo-4H-pyrido[1,2-a]pyrimidin-3-yl)methyl)pyrrolidine-3-carboxylic acid as a yellow solid. (33.6 mg, 13% yield). m/z found: 728.3 [M+H]⁺. ¹H NMR (400 MHz, Methanol-d₄): δ 8.21 (d, J=7.2 Hz, 1H), 8.57 (s, 1H), 8.48 (s, 1H), 7.88 (d, J=1.2 Hz, 1H), 7.74 (dd, J=1.6 Hz, J=7.6 Hz, 1H), 7.68-7.58 (m, 4H), 7.55 (dd, J=2 Hz, J=6.8 Hz, 1H), 7.50 (dd, J=1.2 Hz, J=7.6 Hz, 1H), 4.46 (s, 2H), 4.22 (d, J=2.4 Hz, 2H), 4.10 (m, 3H), 4.00-3.92 (m, 1H), 3.70-3.65 (m, 1H), 3.56-3.43 (m, 3H), 3.19-3.15 (m, 1H), 3.04-2.97 (m, 2H), 2.44-2.28 (m, 5H), 1.96-1.86 (m, 1H).

### Example 117: (S)-1-((8-(2,2′-Dichloro-3′-(6-methoxy-5-((((5-oxopyrrolidin-2-yl)methyl)amino)methyl)pyrazin-2-yl)-[1,1′-biphenyl]-3-yl)-4-oxo-4H-pyrido[1,2-a]pyrimidin-3-yl)methyl)azetidine-3-carboxylic acid

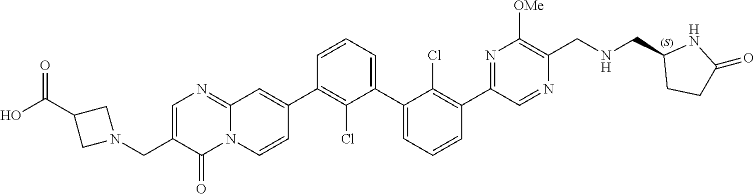

### (S)-1-((8-(3′-(5-(((tert-Butoxycarbonyl)((5-oxopyrrolidin-2-yl)methyl)amino)methyl)-6-methoxypyrazin-2-yl)-2,2′-dichloro-[1,1′-biphenyl]-3-yl)-4-oxo-4H-pyrido[1,2-a]pyrimidin-3-yl)methyl)azetidine-3-carboxylic acid

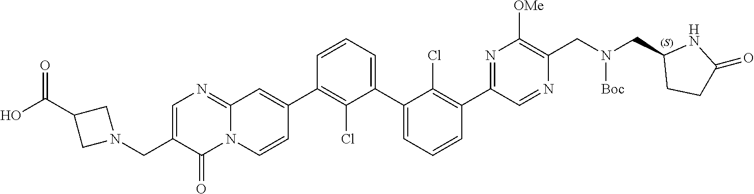

To a mixture (S)-tert-butyl ((5-(2,2′-dichloro-3′-(3-formyl-4-oxo-4H-pyrido[1,2-a]pyrimidin-8-yl)-[1,1′-biphenyl]-3-yl)-3-methoxypyrazin-2-yl)methyl)((5-oxopyrrolidin-2-yl)methyl)carbamate (0.3 g, 0.41 mmol in DCM/MeOH (1:1, 8 mL) was added azetidine-3-carboxylic acid (0.21 g, 2.06 mmol), sodium acetate (0.17 g, 2.06 mmol). The mixture was stirred at r.t. for 10 hr. sodium triacetoxyborohydride (0.44 g, 2.06 mmol) was added. The mixture was stirred at r.t. for 2 hr. The reaction mixture was filtered and concentrated under reduced pressure to give (S)-1-((8-(3′-(5-(((tert-butoxycarbonyl)((5-oxopyrrolidin-2-yl)methyl)amino)methyl)-6-methoxypyrazin-2-yl)-2,2′-dichloro-[1,1′-biphenyl]-3-yl)-4-oxo-4H-pyrido[1,2-a]pyrimidin-3-yl)methyl)azetidine-3-carboxylic acid (0.3 g, crude) as a yellow solid. m/z found: 814.3 [M+H]⁺.

### (S)-1-((8-(2,2′-Dichloro-3′-(6-methoxy-5-((((5-oxopyrrolidin-2-yl)methyl)amino)methyl)pyrazin-2-yl)-[1,1′-biphenyl]-3-yl)-4-oxo-4H-pyrido[1,2-a]pyrimidin-3-yl)methyl)azetidine-3-carboxylic acid

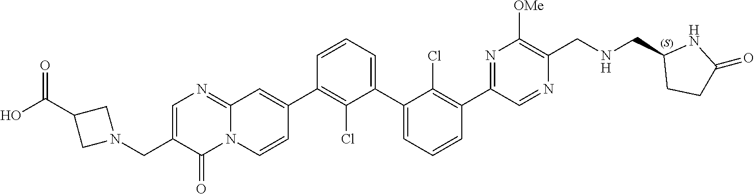

To a mixture 1-[[8-[3-[3-[5-[[tert-butoxycarbonyl-[[(2S)-5-oxopyrrolidin-2-yl]methyl]amino]methyl]-6-methoxy-pyrazin-2-yl]-2-chloro-phenyl]-2-chloro-phenyl]-4-oxo-pyrido[1,2-a]pyrimidin-3-yl]methyl]azetidine-3-carboxylic acid (0.28 g, 0.34 mmol) in DCM (3 mL) was added trifluoroacetic acid (5 mL). After that, the mixture was stirred at r.t. for 0.5 hr. The reaction mixture was concentrated under reduced pressure. The residue was purified by reverse phase HPLC to afford (S)-1-((8-(2,2′-dichloro-3′-(6-methoxy-5-((((5-oxopyrrolidin-2-yl)methyl)amino)methyl)pyrazin-2-yl)-[1,1′-biphenyl]-3-yl)-4-oxo-4H-pyrido[1,2-a]pyrimidin-3-yl)methyl)azetidine-3-carboxylic acid as a white solid (30.4 mg, 12% yield). m/z found: 714.3 [M+H]⁺. ¹H NMR (400 MHz, Methanol-d₄): δ 9.20-9.18 (d, J=7.6 Hz, 1H), 8.54 (s, 1H), 8.50 (s, 1H), 7.87 (s, 1H), 7.74 (dd, J=7.6 Hz, J=1.6 Hz, 1H), 7.67-7.58 (m, 4H), 7.56 (dd, J=6.8 Hz, J=2 Hz, 1H), 7.50 (dd, J=1.2 Hz, J=7.6 Hz, 1H), 4.45 (s, 2H), 4.31-4.23 (m, 6H), 4.11 (s, 3H), 4.00 (m, 1H), 3.50-3.42 (m, 1H), 3.10-3.01 (m, 2H), 2.45-2.39 (m, 3H), 1.94-1.88 (m, 1H).

### Example 118: (S)-1-((6-(2,2′-Dichloro-3′-(4-oxo-3-(((((S)-5-oxopyrrolidin-2-yl)methyl)amino)methyl)-4H-pyrido[1,2-a]pyrimidin-8-yl)-[1,1′-biphenyl]-3-yl)-2-methoxypyridin-3-yl)methyl)pyrrolidine-3-carboxylic acid

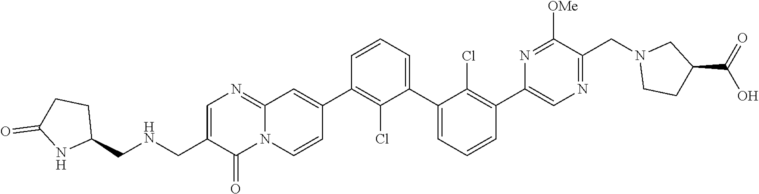 [SMILES: COc1nc(-c2cccc(-c3cccc(-c4ccn5c(=O)c(CNC[C@@H]6CCC(=O)N6)cnc5c4)c3Cl)c2Cl)cnc1CN1CC[C@H](C(=O)O)C1]

### (S)-tert-Butyl ((8-(2,2′-dichloro-3′-(5-formyl-6-methoxypyridin-2-yl)-[1,1′-biphenyl]-3-yl)-4-oxo-4H-pyrido[1,2-a]pyrimidin-3-yl)methyl)((5-oxopyrrolidin-2-yl)methyl)carbamate

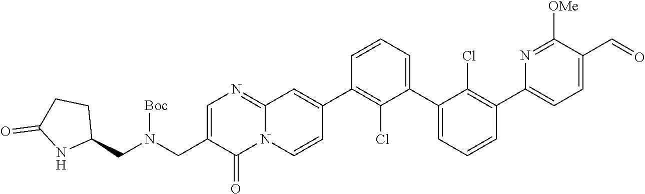 [SMILES: COc1nc(-c2cccc(-c3cccc(-c4ccn5c(=O)c(CN(C[C@@H]6CCC(=O)N6)C(=O)OC(C)(C)C)cnc5c4)c3Cl)c2Cl)ccc1C=O]

To a mixture of (S)-tert-butyl ((8-(3-bromo-2-chlorophenyl)-4-oxo-4H-pyrido[1,2-a]pyrimidin-3-yl)methyl)((5-oxopyrrolidin-2-yl)methyl)carbamate (0.5 g, 0.89 mmol) and 6-(2-chloro-3-(4,4,5,5-tetramethyl-1,3-dioxolan-2-yl)phenyl)-2-methoxynicotinaldehyde (0.74 g, 0.89 mmol, 45% purity) in dioxane/H₂O mixture (5:1, 18 mL) was added potassium phosphate (0.38 g, 1.78 mmol) and [1,1′-bis(di-tert-butylphosphino)ferrocene]dichloropalladium(II) (58 mg, 89 umol). The mixture was stirred at 110° C. for 1 h under N₂. The reaction was concentrated to give the residue. The residue was purified by normal phase SiO₂ chromatography (0-15% EtOAc/petroleum ether) to afford (S)-tert-butyl ((8-(2,2′-dichloro-3′-(5-formyl-6-methoxypyridin-2-yl)-[1,1′-biphenyl]-3-yl)-4-oxo-4H-pyrido[1,2-a]pyrimidin-3-yl)methyl)((5-oxopyrrolidin-2-yl)methyl)carbamate as a brown solid (0.7 g, crude, m/z: 728 [M+H]⁺).

### (S)-1-((6-(3′-(3-(((tert-Butoxycarbonyl)(((S)-5-oxopyrrolidin-2-yl)methyl)amino)methyl)-4-oxo-4H-pyrido[1,2-a]pyrimidin-8-yl)-2,2′-dichloro-[1,1′-biphenyl]-3-yl)-2-methoxypyridin-3-yl)methyl)pyrrolidine-3-carboxylic acid

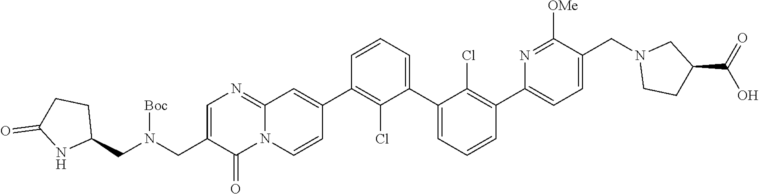 [SMILES: COc1nc(-c2cccc(-c3cccc(-c4ccn5c(=O)c(CN(C[C@@H]6CCC(=O)N6)C(=O)OC(C)(C)C)cnc5c4)c3Cl)c2Cl)ccc1CN1CC[C@H](C(=O)O)C1]

To a mixture of (S)-tert-butyl ((8-(2,2′-dichloro-3′-(5-formyl-6-methoxypyridin-2-yl)-[1,1′-biphenyl]-3-yl)-4-oxo-4H-pyrido[1,2-a]pyrimidin-3-yl)methyl)((5-oxopyrrolidin-2-yl)methyl)carbamate (0.35 g, 0.48 mmol) and (S)-pyrrolidine-3-carboxylic acid (0.16 g, 1.44 mmol) in DCM (5 mL) was added sodium acetate (0.2 g, 2.4 mmol). The mixture was stirred at r.t. for 0.5 h. Sodium triacetyloxyboranuide (0.31 g, 1.44 mmol) was added to the mixture and stirred at r.t. for 2.5 h under N₂. The mixture was filtered. The filtrate was concentrated to afford (S)-1-((6-(3′-(3-(((tert-butoxycarbonyl)(((S)-5-oxopyrrolidin-2-yl)methyl)amino) methyl)-4-oxo-4H-pyrido[1,2-a]pyrimidin-8-yl)-2,2′-dichloro-[1,1′-biphenyl]-3-yl)-2-methoxypyridin-3-yl)methyl)pyrrolidine-3-carboxylic acid as a brown oil. The material was used without further purification (0.5 g, crude, m/z: 827 [M+H]⁺).

### (S)-1-((6-(2,2′-Dichloro-3′-(4-oxo-3-(((((S)-5-oxopyrrolidin-2-yl)methyl)amino)methyl)-4H-pyrido[1,2-a]pyrimidin-8-yl)-[1,1′-biphenyl]-3-yl)-2-methoxypyridin-3-yl)methyl)pyrrolidine-3-carboxylic acid

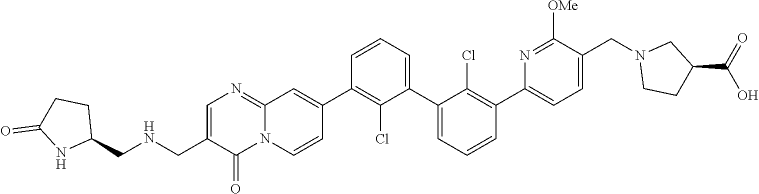 [SMILES: COc1nc(-c2cccc(-c3cccc(-c4ccn5c(=O)c(CNC[C@@H]6CCC(=O)N6)cnc5c4)c3Cl)c2Cl)ccc1CN1CC[C@H](C(=O)O)C1]

To a mixture of (S)-1-((6-(3′-(3-(((tert-butoxycarbonyl)(((S)-5-oxopyrrolidin-2-yl)methyl) amino)methyl)-4-oxo-4H-pyrido[1,2-a]pyrimidin-8-yl)-2,2′-dichloro-[1,1′-biphenyl]-3-yl)-2-methoxypyridin-3-yl)methyl)pyrrolidine-3-carboxylic acid (0.5 g, 0.6 mmol) in DCM (5 mL) was added trifluoroacetic acid (6 mL, 81 mmol). The mixture was stirred at r.t. for 0.5 h under N₂. The reaction mixture was concentrated to give the residue. The residue was purified by reverse phase HPLC to afford (S)-1-((6-(2,2′-dichloro-3′-(4-oxo-3-(((((S)-5-oxopyrrolidin-2-yl)methyl)amino)methyl)-4H-pyrido[1,2-a]pyrimidin-8-yl)-[1,1′-biphenyl]-3-yl)-2-methoxypyridin-3-yl)methyl)pyrrolidine-3-carboxylic acid as a white solid (114.8 mg, 25% yield, m/z: 727 [M+H]⁺). ¹H NMR (400 MHz, Methanol-d₄): δ9.18 (d, J=7.6 Hz, 1H), 8.48 (s, 1H), 7.93 (d, J=7.2 Hz, 1H), 7.83 (d, J=1.2 Hz, 1H), 7.71 (dd, J=7.2 Hz, J=1.6 Hz, 1H), 7.65-7.54 (m, 5H), 7.45 (dd, J=7.2 Hz, J=1.6 Hz, 1H), 7.38 (d, J=7.6 Hz, 1H), 4.45 (s, 2H), 4.14-4.06 (m, 5H), 3.98-3.95 (m, 1H), 3.65-3.61 (m, 1H), 3.48-3.37 (m, 3H), 3.17-3.15 (m, 1H), 3.00-2.92 (m, 2H), 2.41-2.27 (m, 5H), 1.93-1.84 (m, 1H).

### Example 119: (S)-1-((6-(2,2′-Dichloro-3′-(4-oxo-3-((((5-oxopyrrolidin-2-yl)methyl)amino)methyl)-4H-pyrido[1,2-a]pyrimidin-8-yl)-[1,1′-biphenyl]-3-yl)-2-methoxypyridin-3-yl)methyl)azetidine-3-carboxylic acid

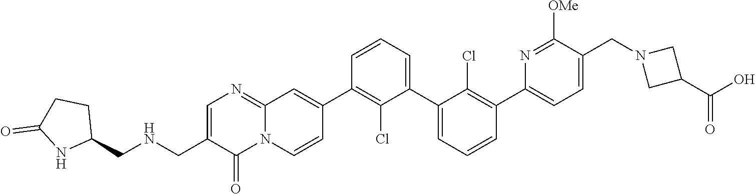 [SMILES: COc1nc(-c2cccc(-c3cccc(-c4ccn5c(=O)c(CNC[C@@H]6CCC(=O)N6)cnc5c4)c3Cl)c2Cl)ccc1CN1CC(C(=O)O)C1]

### (S)-1-((6-(3′-(3-(((tert-Butoxycarbonyl)((5-oxopyrrolidin-2-yl)methyl)amino)methyl)-4-oxo-4H-pyrido[1,2-a]pyrimidin-8-yl)-2,2′-dichloro-[1,1′-biphenyl]-3-yl)-2-methoxypyridin-3-yl)methyl)azetidine-3-carboxylic acid

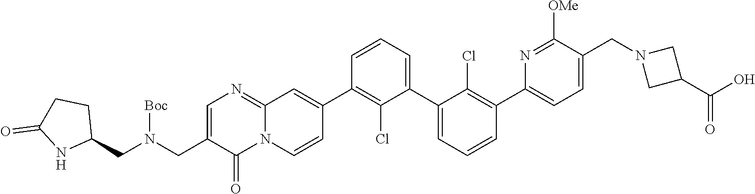 [SMILES: COc1nc(-c2cccc(-c3cccc(-c4ccn5c(=O)c(CN(C[C@@H]6CCC(=O)N6)C(=O)OC(C)(C)C)cnc5c4)c3Cl)c2Cl)ccc1CN1CC(C(=O)O)C1]

To a mixture of (S)-tert-butyl ((8-(2,2′-dichloro-3′-(5-formyl-6-methoxypyridin-2-yl)-[1,1′-biphenyl]-3-yl)-4-oxo-4H-pyrido[1,2-a]pyrimidin-3-yl)methyl)((5-oxopyrrolidin-2-yl)methyl)carbamate (0.3 g, 0.4 mmol) and azetidine-3-carboxylic acid (62 mg, 0.62 mmol) in DCM (5 mL) was added sodium acetate (68 mg, 0.82 mmol). The mixture was stirred at r.t. for 0.5 h under N₂, then sodium triacetyloxyborohydride (0.26 g, 1.24 mmol) was added to the mixture and stirred at r.t. for 2.5 h under N₂. The mixture was filtered. The filtrate was concentrated to afford (S)-1-((6-(3′-(3-(((tert-butoxycarbonyl)((5-oxopyrrolidin-2-yl)methyl)amino)methyl)-4-oxo-4H-pyrido[1,2-a]pyrimidin-8-yl)-2,2′-dichloro-[1,1′-biphenyl]-3-yl)-2-methoxypyridin-3-yl)methyl)azetidine-3-carboxylic acid as a brown oil without purification (0.5 g, crude, m/z: 813 [M+H]⁺).

### (S)-1-((6-(2,2′-Dichloro-3′-(4-oxo-3-((((5-oxopyrrolidin-2-yl)methyl)amino)methyl)-4H-pyrido[1,2-a]pyrimidin-8-yl)-[1,1′-biphenyl]-3-yl)-2-methoxypyridin-3-yl)methyl)azetidine-3-carboxylic acid

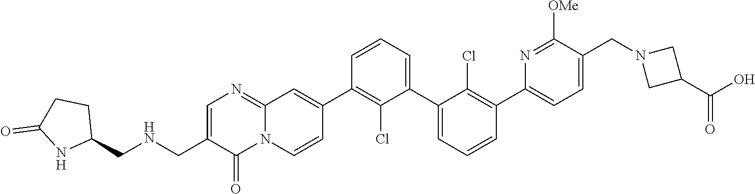 [SMILES: COc1nc(-c2cccc(-c3cccc(-c4ccn5c(=O)c(CNC[C@@H]6CCC(=O)N6)cnc5c4)c3Cl)c2Cl)ccc1CN1CC(C(=O)O)C1]

To a mixture of (S)-1-((6-(3′-(3-(((tert-butoxycarbonyl)((5-oxopyrrolidin-2-yl)methyl)amino)methyl)-4-oxo-4H-pyrido[1,2-a]pyrimidin-8-yl)-2,2′-dichloro-[1,1′-biphenyl]-3-yl)-2-methoxypyridin-3-yl)methyl)azetidine-3-carboxylic acid (0.5 g, 0.62 mmol) in DCM (5 mL) was added trifluoroacetic acid (6 mL, 80 mmol). The mixture was stirred at r.t. for 0.5 h under N₂. The mixture was concentrated to give the residue. The residue was purified by reverse phase HPLC to afford (S)-1-((6-(2,2′-dichloro-3′-(4-oxo-3-((((5-oxopyrrolidin-2-yl)methyl)amino)methyl)-4H-pyrido[1,2-a]pyrimidin-8-yl)-[1,1′-biphenyl]-3-yl)-2-methoxypyridin-3-yl)methyl)azetidine-3-carboxylic acid as a white solid (85.4 mg, 19% yield, m/z: 713 [M+H]⁺). ¹H NMR (400 MHz, Methanol-d₄): δ9.19 (d, J=7.2 Hz, 1H), 8.50 (s, 1H), 7.90 (d, J=7.2 Hz, 1H), 7.85 (d, J=1.2 Hz, 1H), 7.71-7.61 (m, 4H), 7.56-7.54 (m, 2H), 7.45 (dd, J=7.2 Hz, J=2 Hz, 1H), 7.37 (d, J=7.6 Hz, 1H), 4.45 (s, 2H), 4.28-4.26 (m, 4H), 4.15 (d, J=4.4 Hz, 2H), 4.01 (s, 3H), 4.01-3.98 (m, 1H), 3.51-3.42 (m, 1H), 3.09-2.99 (m, 2H), 2.45-2.34 (m, 3H), 1.93-1.86 (m, 1H).

### Example 120: (S)-1-(((8-(2,2′-Dichloro-3′-(6-methoxy-5-((((5-oxopyrrolidin-2-yl)methyl)amino)methyl)-pyridin-2-yl)-[1,1′-biphenyl]-3-yl)-4-oxo-4H-pyrido[1,2-a]pyrimidin-3-yl)methyl)amino)-cyclopropane-1-carboxylic acid

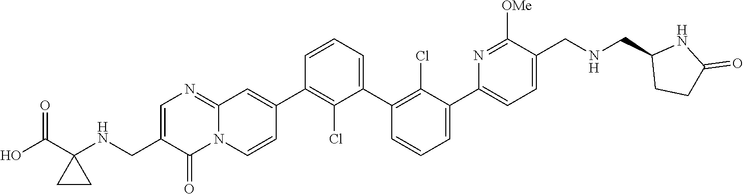 [SMILES: COc1nc(-c2cccc(-c3cccc(-c4ccn5c(=O)c(CNC6(C(=O)O)CC6)cnc5c4)c3Cl)c2Cl)ccc1CNC[C@@H]1CCC(=O)N1]

To a solution of tert-butyl N-[[6-[2-chloro-3-[2-chloro-3-(3-formyl-4-oxo-pyrido[1,2-a]-pyrimidin-8-yl)phenyl]phenyl]-2-methoxy-3-pyridyl]methyl]-N-[[rac-(2S)-5-oxopyrrolidin-2-yl]-methyl]carbamate (50 mg, 0.07 mmol) and methyl 1-aminocyclopropane-1-carboxylate (20 mg, 0.2 mmol) in dioxane (5 mL), acetic acid (10 mg, 0.16 mmol) and stirred for 1 hour at 60° C. and then added sodium triacetoxyborohydride (105 mg, 0.5 mmol) and stirred for 2 h. Reaction mixture was diluted with water (15 mL), extracted with dichloromethane (2×20 mL), combined organic layer was washed with brine (30 mL), dried over sodium sulfate (10 g), solvents evaporated to give methyl 1-[[8-[3-[3-[5-[[tert-butoxycarbonyl-[[rac-(2S)-5-oxopyrrolidin-2-yl]-methyl]-amino]-methyl]-6-methoxy-2-pyridyl]-2-chloro-phenyl]-2-chloro-phenyl]-4-oxo-pyrido-[1,2-a]-pyrimidin-3-yl]-methylamino]-cyclopropanecarboxylate as an yellow solid and used for next reaction without further purification (42 mg, 71.4% yield) m/z: 828 [M+H]⁺ observed.

To the methyl 1-[[8-[3-[3-[5-[[tert-butoxycarbonyl-[[rac-(2S)-5-oxopyrrolidin-2-yl]-methyl]-amino]-methyl]-6-methoxy-2-pyridyl]-2-chloro-phenyl]-2-chloro-phenyl]-4-oxo-pyrido-[1,2-a]-pyrimidin-3-yl]-methylamino]-cyclopropanecarboxylate (42 mg, 0.05 mmol) in dioxane (3 mL), HCl in dioxane (1 mL, 4 mmol, 4 M HCl in dioxane) was added and stirred for 1 hour at room temperature. Solvents were removed and residue was dissolved in dioxane (4 mL), aqueous lithium hydroxide solution (96 mg in 2 ml of water, 4 mmol) was added and stirred at room temperature for 4 hours. Neutralized the reaction mixture with aqueous HCl solution, solvents evaporated and crude was purified by HPLC with eluants 0 to 100% acetonitrile in water (formic acid as modifier) to give (S)-1-(((8-(2,2′-dichloro-3′-(6-methoxy-5-((((5-oxopyrrolidin-2-yl)methyl)-amino)-methyl)-pyri-din-2-yl)-[1,1′-biphenyl]-3-yl)-4-oxo-4H-pyrido[1,2-a]pyrimidin-3-yl)-methyl)-amino)-cyclopro-pane-1-carboxylic acid (as bis-formic acid salt) as a white solid (15 mg, 41.4% yield). m/z: 715 [M+H]⁺ observed. ¹H NMR (400 MHz, D₂O) δ8.57 (brs, 1H), 8.32 (brs, 1H), 8.25 (brs, 1H), 7.60 (brs, 1H), 7.32-6.75 (m, 8H), 4.09-4.01 (m, 6H), 3.12-3.00 (m, 3H), 2.30-2.20 (m, 3H), 1.70-1.65 (m, 1H), 1.27-1.20 (m, 5H).

### Example 121: Methyl (S)-2-((6-(2,2′-dichloro-3′-(4-oxo-3-((((5-oxopyrrolidin-2-yl)methyl)amino)methyl)-4H-pyrido[1,2-a]pyrimidin-8-yl)-[1,1′-biphenyl]-3-yl)-2-methoxypyridin-3-yl)methyl)-2-azabicyclo[2.2.2]octane-4-carboxylate

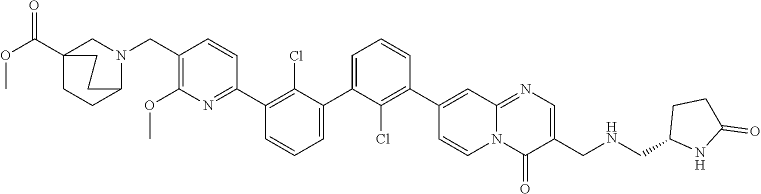 [SMILES: COC(=O)C12CCC(CC1)N(Cc1ccc(-c3cccc(-c4cccc(-c5ccn6c(=O)c(CNC[C@@H]7CCC(=O)N7)cnc6c5)c4Cl)c3Cl)nc1OC)C2]

tert-Butyl N-[[8-[2-chloro-3-[2-chloro-3-(5-formyl-6-methoxy-2-pyridyl)phenyl]phenyl]-4-oxo-pyrido[1,2-a]pyrimidin-3-yl]methyl]-N-[[(2S)-5-oxopyrrolidin-2-yl]methyl]carbamate (30 mg, 0.04 mmol), methyl 2-azabicyclo[2.2.2]octane-4-carboxylate;hydrochloride (25 mg, 0.12 mmol), DIEA (22 μL, 0.12 mmol) and acetic acid (5 mg, 0.08 mmol) were dissolved in 1 ml of 1:1 MeOH/THF solvent mix, and the reaction was stirred at 60° C. for 3 hours. NaBH₃CN (5 mg, 0.08 mmol) was added at 25° C., and the resulting mixture was stirred at rt for 1 hour. The reaction was concentrated, and the residue was dissolved in water (3 ml), then extracted with DCM (3×2 ml). The combined organics were further washed with brine, dried over sodium sulfate, then filtered and concentrated to afford methyl 2-[[6-[3-[3-[3-[[tert-butoxycarbonyl-[[(2S)-5-oxopyrrolidin-2-yl]methyl]amino]methyl]-4-oxo-pyrido[1,2-a]pyrimidin-8-yl]-2-chloro-phenyl]-2-chloro-phenyl]-2-methoxy-3-pyridyl]methyl]-2-azabicyclo[2.2.2]octane-4-carboxylate (36 mg) as an orange oil. Material was used as—is for the next step. MS: m/z found 881, 883 [M+H]⁺.

Methyl 2-[[6-[3-[3-[3-[[tert-butoxycarbonyl-[[(2S)-5-oxopyrrolidin-2-yl]methyl]amino]methyl]-4-oxo-pyrido[1,2-a]pyrimidin-8-yl]-2-chloro-phenyl]-2-chloro-phenyl]-2-methoxy-3-pyridyl]-methyl]-2-azabicyclo[2.2.2]octane-4-carboxylate (36 mg, 0.04 mmol) was dissolved in 0.5 ml DCM and 4M HCl solution in dioxane (51 μL, 7 mg, 0.20 mmol) was added. The reaction was stirred at rt for 20 minutes and the mixture was subsequently concentrated. Crude sample was purified by reverse phase HPLC to afford methyl 2-[[6-[2-chloro-3-[2-chloro-3-[4-oxo-3-[[[(2S)-5-oxopyrrolidin-2-yl]methylamino]methyl]pyrido[1,2-a]pyrimidin-8-yl]phenyl]phenyl]-2-methoxy-3-pyridyl]methyl]-2-azabicyclo[2.2.2]octane-4-carboxylate (11 mg, 34.5% yield) as a light yellow fluffy solid (bis formic acid salt). MS: m/z found 781.4, 783 [M+H]⁺. ¹H NMR (400 MHz, Methanol-d₄) δ 9.13 (d, J=7.5 Hz, 1H), 8.53 (s, 1H), 8.42 (s, 1H), 7.85 (d, J=7.5 Hz, 1H), 7.77 (d, J=1.8 Hz, 1H), 7.67 (dd, J=7.7, 1.7 Hz, 1H), 7.64-7.56 (m, 2H), 7.55-7.48 (m, 3H), 7.40 (dd, J=7.7, 1.7 Hz, 1H), 7.28 (d, J=7.5 Hz, 1H), 4.01 (s, 3H), 3.94 (s, 2H), 3.87 (dd, J=13.3, 5.2 Hz, 3H), 3.65 (s, 3H), 3.07 (s, 2H), 2.90 (s, 1H), 2.81-2.67 (m, 2H), 2.38-2.25 (m, 3H), 2.12 (s, 2H), 1.88 (t, J=8.0 Hz, 4H), 1.69 (s, 2H).

### Example 122: (S)-2-((6-(2,2′-Dichloro-3′-(4-oxo-3-((((5-oxopyrrolidin-2-yl)methyl)amino)methyl)-4H-pyrido[1,2-a]pyrimidin-8-yl)-[1,1′-biphenyl]-3-yl)-2-methoxypyridin-3-yl)methyl)-2-azabicyclo[2.2.2]octane-4-carboxylic acid

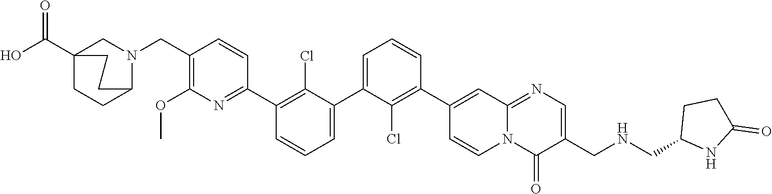 [SMILES: COc1nc(-c2cccc(-c3cccc(-c4ccn5c(=O)c(CNC[C@@H]6CCC(=O)N6)cnc5c4)c3Cl)c2Cl)ccc1CN1CC2(C(=O)O)CCC1CC2]

Methyl 2-[[6-[2-chloro-3-[2-chloro-3-[4-oxo-3-[[[(2S)-5-oxopyrrolidin-2-yl]methylamino]methyl]pyrido[1,2-a]pyrimidin-8-yl]phenyl]phenyl]-2-methoxy-3-pyridyl]methyl]-2-azabicyclo[2.2.2]octane-4-carboxylate (9 mg, 0.01 mmol) was dissolved in 0.5 ml THE and 0.5 mL of 1M aqueous NaOH solution was added. Reaction mixture was stirred at rt for 8 hours, then the solution was concentrated. Crude sample was purified by reverse phase HPLC to afford 2-[[6-[2-chloro-3-[2-chloro-3-[4-oxo-3-[[[(2S)-5-oxopyrrolidin-2-yl]methylamino]methyl]pyrido[1,2-a]pyrimidin-8-yl]phenyl]phenyl]-2-methoxy-3-pyridyl]methyl]-2-azabicyclo[2.2.2]octane-4-carboxylic acid (2.2 mg, 24.9% yield) as a white fluffy solid (bis formic acid salts). MS: m/z found 767.3, 769 [M+H]⁺. ¹H NMR (400 MHz, Methanol-d₄) δ 9.14 (d, J=7.4 Hz, 1H), 8.50 (s, 2H), 8.43 (s, 1H), 7.97 (d, J=7.7 Hz, 1H), 7.78 (d, J=1.8 Hz, 1H), 7.69 (dd, J=7.8, 1.7 Hz, 1H), 7.66-7.58 (m, 2H), 7.57-7.50 (m, 3H), 7.44 (dd, J=7.6, 1.7 Hz, 1H), 7.39 (d, J=7.5 Hz, 1H), 4.46 (s, 2H), 4.08 (s, 3H), 3.91 (dd, J=18.4, 5.0 Hz, 3H), 3.41 (s, 2H), 2.80 (qd, J=12.2, 6.1 Hz, 2H), 2.34 (dt, J=14.4, 7.5 Hz, 3H), 1.99 (d, J=13.0 Hz, 2H), 1.87 (dd, J=20.0, 8.5 Hz, 5H).

### Example 123: (S)-5-((((3-Chloro-6-(2,2′-dichloro-3′-(6-methoxy-5-(((((S)-5-oxopyrrolidin-2-yl)methyl)amino)methyl)pyridin-2-yl)-[1,1′-biphenyl]-3-yl)thieno[3,2-b]pyridin-2-yl)methyl)amino)methyl)pyrrolidin-2-one

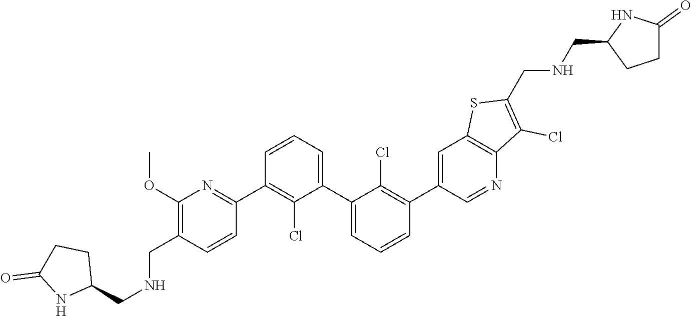 [SMILES: COc1nc(-c2cccc(-c3cccc(-c4cnc5c(Cl)c(CNC[C@@H]6CCC(=O)N6)sc5c4)c3Cl)c2Cl)ccc1CNC[C@@H]1CCC(=O)N1]

### methyl 6-bromo-3-chlorothieno[3,2-b]-pyridine-2-carboxylate

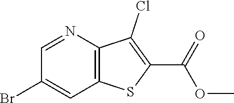

Methyl 3-amino-6-bromo-thieno[3,2-b]pyridine-2-carboxylate (0.50 g, 1.74 mmol), CuCl (0.34 g, 3.48 mmol) and CuCl₂ (0.69 g, 5.22 mmol) were suspended in 9 ml anhydrous acetonitrile and the mixture was stirred at rt for 10 minutes. tert-butyl nitrite (0.62 mL, 0.54 g, 5.22 mmol) was added slowly over 5 minutes, and the reaction was stirred at rt for an additional 3 hours. 30 mL of 1M aqueous HCl solution was slowly added to the reaction and the resulting solution was extracted with EtOAc (3×20 mL). The combined organics were washed with water, brine, dried over sodium sulfate, then filtered and concentrated. The crude sample was suspended in 30 mL of 9:1 hexane/EtOAc and the mixture was sonicated. The precipitate was filtered and dried to afford methyl 6-bromo-3-chloro-thieno[3,2-b]-pyridine-2-carboxylate (0.38 g, 71% yield) as a tan solid. MS: m/z found 307, 309 [M+H]⁺.

### (6-Bromo-3-chlorothieno[3,2-b]-pyridin-2-yl)-methanol

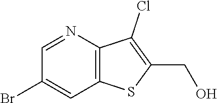 [SMILES: OCc1sc2cc(Br)cnc2c1Cl]

Methyl 6-bromo-3-chloro-thieno[3,2-b]-pyridine-2-carboxylate (0.38 g, 1.24 mmol) was dissolved in 10 ml DCM and the solution was cooled to −10° C. A solution of 1M di-isobutylaluminum hydride in THF, (2.96 mL, 2.86 mmol) was added dropwise over 15 minutes, and the resulting mixture was stirred at −10° C. for 1 hour. The reaction was then warmed to rt and stirred for an additional 3 hours. The reaction was subsequently diluted with 20 mL DCM and the solution was washed with 1M HCl solution (2×20 mL). The organic layer was further washed with brine (20m), dried over sodium sulfate, then filtered and concentrated to afford (6-bromo-3-chloro-thieno[3,2-b]pyridin-2-yl)methanol (0.35 g) as a light yellow foam. Material was used as—is for the next step. MS: m/z found 278, 280 [M+H]⁺.

### 6-Bromo-3-chlorothieno[3,2-b]-pyridine-2-carbaldehyde

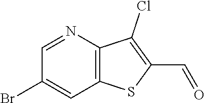 [SMILES: O=Cc1sc2cc(Br)cnc2c1Cl]

Dess-Martin periodinane (1.05 g, 2.48 mmol) was added portion-wise to a solution of (6-bromo-3-chloro-thieno[3,2-b]pyridin-2-yl)methanol (0.35 g, 1.24 mmol) in 20 ml DCM/1 mL DMF and the mixture was stirred at rt for 2 hours. The reaction was then concentrated to remove all the DCM and the remaining solution was diluted with EtOAc (30 mL). The precipitate was filtered and the EtOAc filtrate was concentrated. Crude sample was purified by silica gel column chromatography, eluting with 0-15% EtOAc gradient in hexane to afford 6-bromo-3-chloro-thieno[3,2-b]-pyridine-2-carbaldehyde (65 mg, 18.9% yield) as a white solid. MS: m/z found 276, 278 [M+H]⁺. ¹H NMR (400 MHz, Chloroform-d) δ 10.43 (d, J=0.8 Hz, 1H), 8.90 (dd, J=2.1, 0.8 Hz, 1H), 8.41 (dd, J=2.0, 0.7 Hz, 1H).

### 3-Chloro-6-(2,2′-dichloro-3′-(5-formyl-6-methoxypyridin-2-yl)-[1,1′-biphenyl]-3-yl)-thieno[3,2-b]-pyridine-2-carbaldehyde

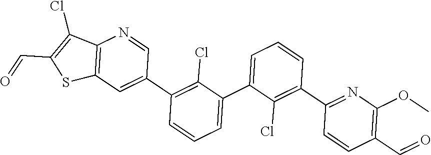 [SMILES: COc1nc(-c2cccc(-c3cccc(-c4cnc5c(Cl)c(C=O)sc5c4)c3Cl)c2Cl)ccc1C=O]

Pd(PPh₃)₄ (20 mg, 0.02 mmol), potassium carbonate (36 mg, 0.26 mmol), 6-bromo-3-chloro-thieno[3,2-b]pyridine-2-carbaldehyde (24 mg, 0.09 mmol) and 6-[2-chloro-3-[2-chloro-3-(4,4,5,5-tetramethyl-1,3,2-dioxaborolan-2-yl)phenyl]phenyl]-2-methoxy-pyridine-3-carbaldehyde (42 mg, 0.09 mmol) were suspended in 1 mL of 4:1 dioxane/water solvent mixture, and the solution was heated at 105° C. for 20 minutes. The reaction was cooled to rt, diluted with 5 mL water, and extracted with EtOAc (3×3 mL). The combined organics were concentrated and crude product was purified by silica gel chromatography, eluting with 0-5% MeOH gradient in DCM to afford 3-chloro-6-[2-chloro-3-[2-chloro-3-(5-formyl-6-methoxy-2-pyridyl)phenyl]phenyl]thieno[3,2-b]pyridine-2-carbaldehyde (24 mg, 50% yield) as a white foam. MS: m/z found 553, 555 [M+H]⁺.

### (S)-5-((((3-Chloro-6-(2,2′-dichloro-3′-(6-methoxy-5-(((((S)-5-oxopyrrolidin-2-yl)methyl)amino)methyl)pyridin-2-yl)-[1,1′-biphenyl]-3-yl)thieno[3,2-b]pyridin-2-yl)methyl)amino)methyl)pyrrolidin-2-one

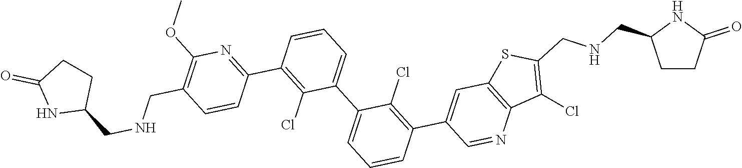 [SMILES: COc1nc(-c2cccc(-c3cccc(-c4cnc5c(Cl)c(CNC[C@@H]6CCC(=O)N6)sc5c4)c3Cl)c2Cl)ccc1CNC[C@@H]1CCC(=O)N1]

3-chloro-6-[2-chloro-3-[2-chloro-3-(5-formyl-6-methoxy-2-pyridyl)phenyl]phenyl]thieno[3,2-b]pyridine-2-carbaldehyde (24 mg, 0.04 mmol), (5S)-5-(aminomethyl)-pyrrolidin-2-one (15 mg, 0.13 mmol) and acetic acid (6.5 mg, 0.11 mmol) were dissolved in 1 ml of 1:1 MeOH/THF solvent mix, and the reaction was stirred at 25° C. for 3 hours. NaBH₃CN (8 mg, 0.13 mmol) was then added portion-wise, and the resulting mixture was stirred at 25° C. for 1 hour. The reaction was concentrated and the crude sample was purified by reverse phase HPLC to afford (5S)-5-[[[6-[2-chloro-3-[2-chloro-3-[3-chloro-2-[[[(2S)-5-oxopyrrolidin-2-yl]methylamino]methyl]thieno[3,2-b]pyridin-6-yl]phenyl]phenyl]-2-methoxy-3-pyridyl]methylamino]methyl]pyrrolidin-2-one (21.7 mg, 66.8%) as yield a white fluffy solid (bis formic acid salt). MS: m/z found 750, 752 [M+H]⁺. ¹H NMR (400 MHz, Methanol-d₄) δ 8.73 (d, J=1.9 Hz, 1H), 8.49 (d, J=1.9 Hz, 1H), 8.40 (s, 1H), 7.80 (d, J=7.5 Hz, 1H), 7.65 (dd, J=7.7, 1.7 Hz, 1H), 7.57-7.54 (m, 2H), 7.51 (t, J=7.7 Hz, 1H), 7.42 (ddd, J=12.9, 6.5, 2.7 Hz, 2H), 7.30 (d, J=7.5 Hz, 1H), 4.25 (d, J=0.9 Hz, 2H), 4.06 (d, J=6.5 Hz, 5H), 3.95 (d, J=6.8 Hz, 1H), 3.83 (p, J=6.2, 5.7 Hz, 1H), 2.95 (h, J=7.1 Hz, 2H), 2.84-2.69 (m, 2H), 2.44-2.20 (m, 7H), 1.95-1.79 (m, 2H).

### Example 124: Methyl (S)-((6-(2,2′-dichloro-3′-(4-oxo-3-((((5-oxopyrrolidin-2-yl)methyl)amino)methyl)-4H-pyrido[1,2-a]pyrimidin-8-yl)-[1,1′-biphenyl]-3-yl)-2-methoxypyridin-3-yl)methyl)glycinate

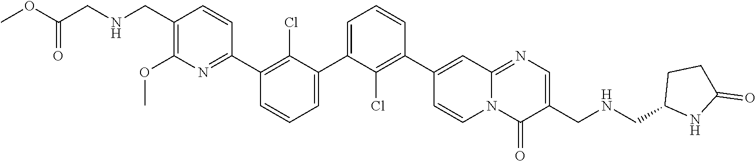 [SMILES: COC(=O)CNCc1ccc(-c2cccc(-c3cccc(-c4ccn5c(=O)c(CNC[C@@H]6CCC(=O)N6)cnc5c4)c3Cl)c2Cl)nc1OC]

tert-butyl N-[[8-[2-chloro-3-[2-chloro-3-(5-formyl-6-methoxy-2-pyridyl)phenyl]phenyl]-4-oxo-pyrido[1,2-a]pyrimidin-3-yl]methyl]-N-[[(2S)-5-oxopyrrolidin-2-yl]methyl]carbamate (30 mg, 0.04 mmol), methyl 2-aminoacetate hydrochloride (15.5 mg, 0.12 mmol), DIEA (22 μL, 0.02 g, 0.12 mmol) and acetic acid (5 mg, 0.08 mmol) were dissolved in 1 ml of 1:1 MeOH/THF solvent mix, and the reaction was stirred at 25° C. for 3 hours. NaBH₃CN (5 mg, 0.08 mmol) was added portion-wise, and the resulting mixture was stirred at 25° C. for 1 hour. The reaction was concentrated and the residue was dissolved in water (3 mL), then extracted with DCM (3×2 mL). The combined organics were further washed with brine, dried over sodium sulfate, then filtered and concentrated to afford methyl 2-[[6-[3-[3-[3-[[tert-butoxycarbonyl-[[(2S)-5-oxopyrrolidin-2-yl]methyl]amino]methyl]-4-oxo-pyrido[1,2-a]pyrimidin-8-yl]-2-chloro-phenyl]-2-chloro-phenyl]-2-methoxy-3-pyridyl]methylamino]acetate (33.00 mg) as a yellow solid. Material was used as—is for the next step. MS: m/z found 801.4, 803 [M+H]⁺.

Methyl 2-[[6-[3-[3-[3-[[tert-butoxycarbonyl-[[(2S)-5-oxopyrrolidin-2-yl]methyl]amino]methyl]-4-oxo-pyrido[1,2-a]pyrimidin-8-yl]-2-chloro-phenyl]-2-chloro-phenyl]-2-methoxy-3-pyridyl]methylamino]acetate (33 mg, 0.04 mmol) was dissolved in 0.5 ml DCM and 4M HCl solution in dioxane (41 μL, 5.93 mg, 0.16 mmol) was added. The reaction was stirred at rt for 20 minutes and the mixture was subsequently concentrated. Crude sample was purified by reverse phase HPLC to afford methyl 2-[[6-[2-chloro-3-[2-chloro-3-[4-oxo-3-[[[(2S)-5-oxopyrrolidin-2-yl]methylamino]methyl]pyrido[1,2-a]pyrimidin-8-yl]phenyl]phenyl]-2-methoxy-3-pyridyl]methylamino]acetate (10.4 mg, 36% yield) as a white fluffy solid (bis formic acid salt). MS: m/z found 701.4, 703.4 [M+H]⁺. ¹H NMR (400 MHz, Methanol-d₄) δ 9.13 (d, J=7.4 Hz, 1H), 8.42 (s, 1H), 7.77 (d, J=1.8 Hz, 1H), 7.71 (d, J=7.4 Hz, 1H), 7.68-7.56 (m, 3H), 7.55-7.48 (m, 3H), 7.39 (dd, J=7.7, 1.7 Hz, 1H), 7.23 (d, J=7.4 Hz, 1H), 4.01 (s, 3H), 3.96-3.82 (m, 5H), 3.71 (s, 3H), 3.46 (s, 2H), 2.79 (qd, J=12.2, 6.1 Hz, 2H), 2.40-2.23 (m, 3H), 1.82 (dq, J=11.9, 7.2, 6.4 Hz, 1H).

### Example 125: (S)-((6-(2,2′-Dichloro-3′-(4-oxo-3-((((5-oxopyrrolidin-2-yl)methyl)amino)methyl)-4H-pyrido[1,2-a]pyrimidin-8-yl)-[1,1′-biphenyl]-3-yl)-2-methoxypyridin-3-yl)methyl)glycine

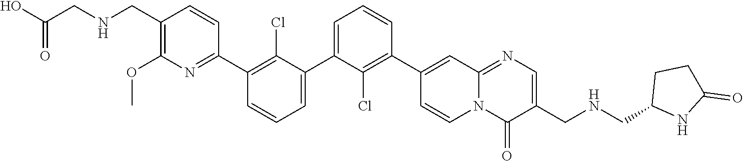 [SMILES: COc1nc(-c2cccc(-c3cccc(-c4ccn5c(=O)c(CNC[C@@H]6CCC(=O)N6)cnc5c4)c3Cl)c2Cl)ccc1CNCC(=O)O]

Methyl 2-[[6-[2-chloro-3-[2-chloro-3-[4-oxo-3-[[[(2S)-5-oxopyrrolidin-2-yl]methylamino]methyl]pyrido[1,2-a]pyrimidin-8-yl]phenyl]phenyl]-2-methoxy-3-pyridyl]methylamino]acetate (9 mg, 0.01 mmol) was dissolved in 0.5 ml THE and hydroxylithium;hydrate (1.6 mg, 0.04 mmol) in 0.1 ml water was added. The resulting mixture was stirred at rt for 1 hour. The reaction was concentrated and the crude sample was purified by reverse phase HPLC to afford 2-[[6-[2-chloro-3-[2-chloro-3-[4-oxo-3-[[[(2S)-5-oxopyrrolidin-2-yl]methylamino]methyl]pyrido[1,2-a]pyrimidin-8-yl]phenyl]phenyl]-2-methoxy-3-pyridyl]methylamino]acetic acid (6.8 mg, 78.0% yield) as a white fluffy solid (bis formic acid salt). MS: m/z found 687.35, 689.3 [M+H]⁺. ¹H NMR (400 MHz, Methanol-d₄) δ 9.20-9.08 (m, 1H), 8.46 (s, 1H), 8.42 (s, 2H), 7.86 (d, J=7.6 Hz, 1H), 7.81 (dd, J=1.9, 0.8 Hz, 1H), 7.67 (dd, J=7.7, 1.7 Hz, 1H), 7.63-7.50 (m, 5H), 7.42 (dd, J=7.7, 1.7 Hz, 1H), 7.33 (d, J=7.5 Hz, 1H), 4.29 (s, 2H), 4.08 (d, J=5.5 Hz, 5H), 3.95 (s, 1H), 3.54 (s, 2H), 2.96 (qd, J=12.4, 6.2 Hz, 2H), 2.44-2.28 (m, 3H), 1.94-1.79 (m, 1H).

### Example 126: (S)-4-(((8-(2,2′-Dichloro-3′-(6-methoxy-5-(((((S)-5-oxopyrrolidin-2-yl)methyl)amino)methyl)pyridin-2-yl)-[1,1′-biphenyl]-3-yl)-4-oxo-4H-pyrido[1,2-a]pyrimidin-3-yl)methyl)amino)-3-hydroxybutanoic acid

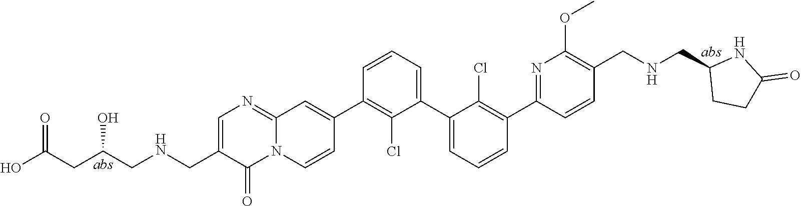 [SMILES: COc1nc(-c2cccc(-c3cccc(-c4ccn5c(=O)c(CNC[C@@H](O)CC(=O)O)cnc5c4)c3Cl)c2Cl)ccc1CNC[C@@H]1CCC(=O)N1]

### (5S)-5-[[[6-(3-Bromo-2-chloro-phenyl)-2-methoxy-3-pyridyl]methylamino]methyl]pyrrolidin-2-one

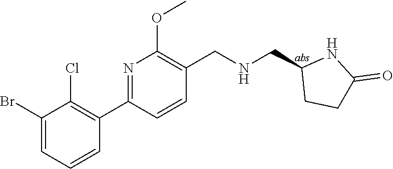 [SMILES: COc1nc(-c2cccc(Br)c2Cl)ccc1CNC[C@@H]1CCC(=O)N1]

A mixture of 6-(3-bromo-2-chloro-phenyl)-2-methoxy-pyridine-3-carbaldehyde (340.00 mg, 1.04 mmol), (5S)-5-(aminomethyl)pyrrolidin-2-one (0.24 g, 2.08 mmol) and acetic acid (60 uL, 63 mg, 1.04 mmol) in MeOH/THF (1:1) was stirred for 1 hour at rt. NaBH₃CN (0.13 g, 2.08 mmol) was added, the mixture was stirred for additional 5 hrs, quenched with water, and extracted into DCM three times. The combined organic layers were dried over anhydrous sodium sulfate, concentrated, and used in the next step without any further purification. MS: m/z found 424.0 [M+H]⁺.

### tert-Butyl N-[[6-(3-bromo-2-chloro-phenyl)-2-methoxy-3-pyridyl]methyl]-N-[[(2S)-5-oxopyrrolidin-2-yl]methyl]carbamate

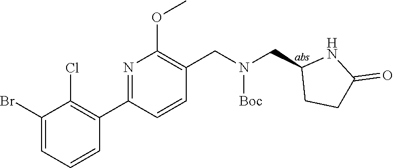 [SMILES: COc1nc(-c2cccc(Br)c2Cl)ccc1CN(C[C@@H]1CCC(=O)N1)C(=O)OC(C)(C)C]

A mixture of (5S)-5-[[[6-(3-bromo-2-chloro-phenyl)-2-methoxy-3-pyridyl]methylamino]methyl]pyrrolidin-2-one (0.41 g, 0.97 mmol) and di-tert-butyl dicarbonate (0.25 g, 1.16 mmol) in DCM was stirred at rt overnight, concentrated and purified on silica gel column using MeOH in DCM (0 to 10% gradient) or EtOAc to give tert-butyl N-[[6-(3-bromo-2-chloro-phenyl)-2-methoxy-3-pyridyl]methyl]-N-[[(2S)-5-oxopyrrolidin-2-yl]methyl]carbamate (0.49 g, 96.7%). MS: m/z found 524.1 [M+H]⁺.

### tert-Butyl N-[[6-[2-chloro-3-[2-chloro-3-(3-formyl-4-oxo-pyrido[1,2-a]pyrimidin-8-yl)phenyl]phenyl]-2-methoxy-3-pyridyl]methyl]-N-[[(2S)-5-oxopyrrolidin-2-yl]methyl]carbamate

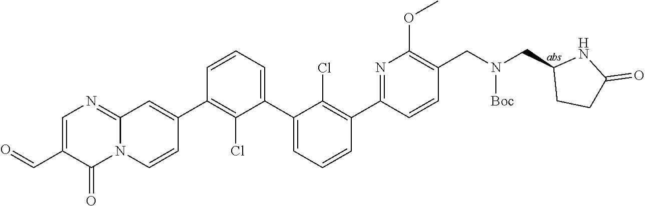 [SMILES: COc1nc(-c2cccc(-c3cccc(-c4ccn5c(=O)c(C=O)cnc5c4)c3Cl)c2Cl)ccc1CN(C[C@@H]1CCC(=O)N1)C(=O)OC(C)(C)C]

To a solution of 8-[2-chloro-3-(4,4,5,5-tetramethyl-1,3,2-dioxaborolan-2-yl)phenyl]-4-oxo-pyrido[1,2-a]pyrimidine-3-carbaldehyde (0.40 g, 0.97 mmol), tert-butyl N-[[6-(3-bromo-2-chloro-phenyl)-2-methoxy-3-pyridyl]methyl]-N-[[(2S)-5-oxopyrrolidin-2-yl]methyl]carbamate (0.56 g, 1.07 mmol) and potassium carbonate (0.40 g, 2.92 mmol) in degassed dioxane/water (6:1) was added Pd(PPh₃)₄ (0.11 g mg, 0.10 mmol) and the mixture was stirred at 90° C. for 4 hours under N₂(g). Water was added and the mixture was extracted into EtOAc and DCM three times. The combined organic layers were dried over anhydrous sodium sulfate, concentrated, and purified on silica gel column using MeOH in DCM (0 to 4% gradient) to obtain tert-butyl N-[[6-[2-chloro-3-[2-chloro-3-(3-formyl-4-oxo-pyrido[1,2-a]pyrimidin-8-yl)phenyl]phenyl]-2-methoxy-3-pyridyl]methyl]-N-[[(2S)-5-oxopyrrolidin-2-yl]methyl]carbamate (0.42 g, 59.2%). MS: m/z found 728.2 [M+H]⁺.

### (3S)-4-[[8-[3-[3-[5-[[tert-Butoxycarbonyl-[[(2S)-5-oxopyrrolidin-2-yl]methyl]amino]methyl]-6-methoxy-2-pyridyl]-2-chloro-phenyl]-2-chloro-phenyl]-4-oxo-pyrido[1,2-a]pyrimidin-3-yl]methylamino]-3-hydroxy-butanoic acid

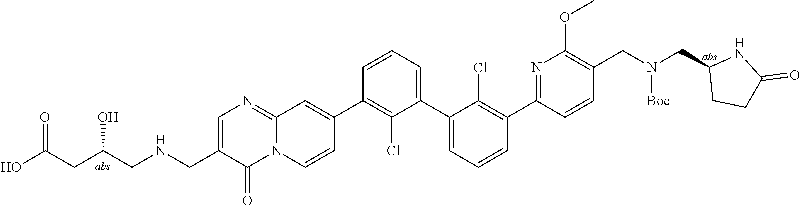 [SMILES: COc1nc(-c2cccc(-c3cccc(-c4ccn5c(=O)c(CNC[C@@H](O)CC(=O)O)cnc5c4)c3Cl)c2Cl)ccc1CN(C[C@@H]1CCC(=O)N1)C(=O)OC(C)(C)C]

A mixture of tert-butyl N-[[6-[2-chloro-3-[2-chloro-3-(3-formyl-4-oxo-pyrido[1,2-a]pyrimidin-8-yl)phenyl]phenyl]-2-methoxy-3-pyridyl]methyl]-N-[[(2S)-5-oxopyrrolidin-2-yl]methyl]carbamate (30 mg, 0.04 mmol), (3S)-4-amino-3-hydroxy-butanoic acid (10 mg, 0.08 mmol) and acetic acid (2 uL, 2.5 mg, 0.04 mmol) in MeOH/THF (1:1) was stirred for 30 min at rt. NaBH₃CN (5 mg, 0.08 mmol) was added, the mixture was stirred overnight at rt. The purification by reversed phase chromatography provided (3S)-4-[[8-[3-[3-[5-[[tert-butoxycarbonyl-[[(2S)-5-oxopyrrolidin-2-yl]methyl]amino]methyl]-6-methoxy-2-pyridyl]-2-chloro-phenyl]-2-chloro-phenyl]-4-oxo-pyrido[1,2-a]pyrimidin-3-yl]methylamino]-3-hydroxy-butanoic acid (15 mg, 43.8%). MS: m/z found 831.3 [M+H]⁺.

### (3S)-4-[[8-[2-Chloro-3-[2-chloro-3-[6-methoxy-5-[[[(2S)-5-oxopyrrolidin-2-yl]methylamino]methyl]-2-pyridyl]phenyl]phenyl]-4-oxo-pyrido[1,2-a]pyrimidin-3-yl]methylamino]-3-hydroxy-butanoic acid

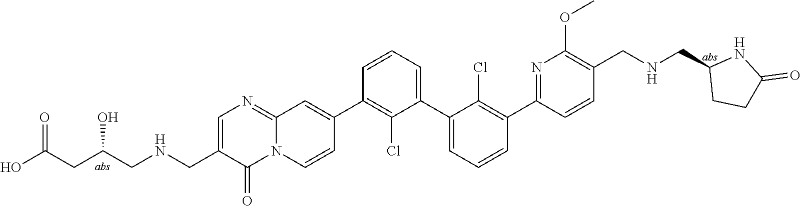 [SMILES: COc1nc(-c2cccc(-c3cccc(-c4ccn5c(=O)c(CNC[C@@H](O)CC(=O)O)cnc5c4)c3Cl)c2Cl)ccc1CNC[C@@H]1CCC(=O)N1]

To a solution of (3S)-4-[[8-[3-[3-[5-[[tert-butoxycarbonyl-[[(2S)-5-oxopyrrolidin-2-yl]methyl]amino]methyl]-6-methoxy-2-pyridyl]-2-chloro-phenyl]-2-chloro-phenyl]-4-oxo-pyrido[1,2-a]pyrimidin-3-yl]methylamino]-3-hydroxy-butanoic acid (13 mg, 0.02 mmol) in DCM was added 4M HCl in dioxane (3 equivalent). The mixture was stirred for 1 hour, concentrated and purified by reversed phase chromatography to give (3S)-4-[[8-[2-chloro-3-[2-chloro-3-[6-methoxy-5-[[[(2S)-5-oxopyrrolidin-2-yl]methylamino]methyl]-2-pyridyl]phenyl]phenyl]-4-oxo-pyrido[1,2-a]pyrimidin-3-yl]methylamino]-3-hydroxy-butanoic acid (10 mg, 87.4%). MS: m/z found 731.3 [M+H]⁺. ¹H NMR (400 MHz, Methanol-d₄): δ 9.18 (dt, J=7.4, 1.0 Hz, 1H), 8.52 (d, J=1.0 Hz, 1H), 8.45 (d, J=1.0 Hz, 1H), 7.85 (dt, J=1.9, 0.9 Hz, 1H), 7.79 (d, J=7.5 Hz, 1H), 7.67 (dd, J=7.7, 1.7 Hz, 1H), 7.65-7.58 (m, 3H), 7.54-7.49 (m, 2H), 7.41 (dt, J=7.6, 1.4 Hz, 1H), 7.29 (dd, J=7.4, 1.0 Hz, 1H), 4.31 (s, 2H), 4.21 (dt, J=9.7, 4.7 Hz, 1H), 4.04 (s, 3H), 4.00 (d, J=3.2 Hz, 2H), 3.95-3.87 (m, 1H), 3.26 (dd, J=12.7, 3.7 Hz, 1H), 3.06 (dd, J=12.6, 8.4 Hz, 1H), 2.93-2.81 (m, 2H), 2.46 (d, J=6.2 Hz, 2H), 2.39-2.28 (m, 3H), 1.90-1.79 (m, 1H).

### Example 127: (S)-2-((8-(2,2′-Dichloro-3′-(6-methoxy-5-((((5-oxopyrrolidin-2-yl)methyl)amino)methyl)pyridin-2-yl)-[1,1′-biphenyl]-3-yl)-4-oxo-4H-pyrido[1,2-a]pyrimidin-3-yl)methyl)-2-azabicyclo[2.2.2]octane-4-carboxylic acid

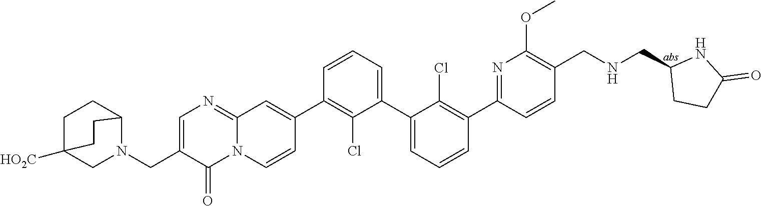 [SMILES: COc1nc(-c2cccc(-c3cccc(-c4ccn5c(=O)c(CN6CC7(C(=O)O)CCC6CC7)cnc5c4)c3Cl)c2Cl)ccc1CNC[C@@H]1CCC(=O)N1]

### 2-[[8-[3-[3-[5-[[tert-Butoxycarbonyl-[[(2S)-5-oxopyrrolidin-2-yl]methyl]amino]methyl]-6-methoxy-2-pyridyl]-2-chloro-phenyl]-2-chloro-phenyl]-4-oxo-pyrido[1,2-a]pyrimidin-3-yl]methyl]-2-azabicyclo[2.2.2]octane-4-carboxylic acid

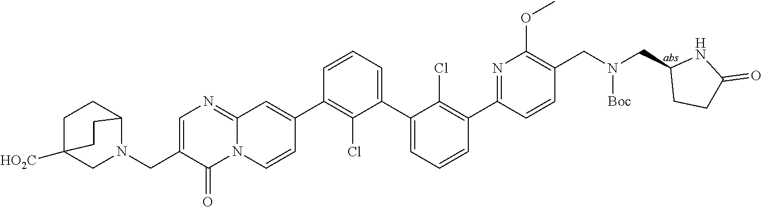 [SMILES: COc1nc(-c2cccc(-c3cccc(-c4ccn5c(=O)c(CN6CC7(C(=O)O)CCC6CC7)cnc5c4)c3Cl)c2Cl)ccc1CN(C[C@@H]1CCC(=O)N1)C(=O)OC(C)(C)C]

A mixture of tert-butyl N-[[6-[2-chloro-3-[2-chloro-3-(3-formyl-4-oxo-pyrido[1,2-a]pyrimidin-8-yl)phenyl]phenyl]-2-methoxy-3-pyridyl]methyl]-N-[[(2S)-5-oxopyrrolidin-2-yl]methyl]carbamate (50 mg, 0.07 mmol), 2-azabicyclo[2.2.2]octane-4-carboxylic acid (21 mg, 0.14 mmol) and acetic acid (4 uL, 4 mg, 0.07 mmol) in MeOH/THF (2:1) was stirred for 30 min at rt. NaBH₃CN (9 mg, 0.14 mmol) was added, the mixture was stirred for 2 hours at rt and purified by reversed phase chromatography to provide 2-[[8-[3-[3-[5-[[tert-butoxycarbonyl-[[(2S)-5-oxopyrrolidin-2-yl]methyl]amino]methyl]-6-methoxy-2-pyridyl]-2-chloro-phenyl]-2-chloro-phenyl]-4-oxo-pyrido[1,2-a]pyrimidin-3-yl]methyl]-2-azabicyclo[2.2.2]octane-4-carboxylic acid (35 mg, 58.8%). MS: m/z found 867.3 [M+H]⁺.

### 2-[[8-[2-chloro-3-[2-chloro-3-[6-methoxy-5-[[[(2S)-5-oxopyrrolidin-2-yl]methylamino]methyl]-2-pyridyl]phenyl]phenyl]-4-oxo-pyrido[1,2-a]pyrimidin-3-yl]methyl]-2-azabicyclo[2.2.2]octane-4-carboxylic acid

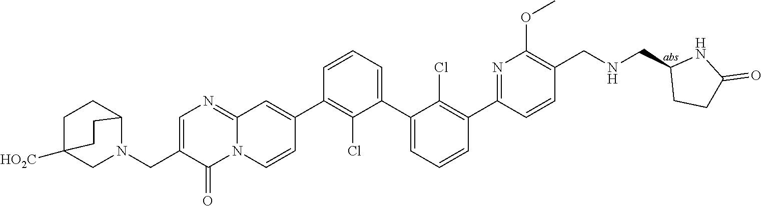 [SMILES: COc1nc(-c2cccc(-c3cccc(-c4ccn5c(=O)c(CN6CC7(C(=O)O)CCC6CC7)cnc5c4)c3Cl)c2Cl)ccc1CNC[C@@H]1CCC(=O)N1]

A solution of 2-[[8-[3-[3-[5-[[tert-butoxycarbonyl-[[(2S)-5-oxopyrrolidin-2-yl]methyl]amino]methyl]-6-methoxy-2-pyridyl]-2-chloro-phenyl]-2-chloro-phenyl]-4-oxo-pyrido[1,2-a]pyrimidin-3-yl]methyl]-2-azabicyclo[2.2.2]octane-4-carboxylic acid (35 mg, 0.04 mmol) in DCM/TFA (2:1) was stirred for 30 minutes, concentrated and purified by reversed phase chromatography to give 2-[[8-[2-chloro-3-[2-chloro-3-[6-methoxy-5-[[[(2S)-5-oxopyrrolidin-2-yl]methylamino]methyl]-2-pyridyl]phenyl]phenyl]-4-oxo-pyrido[1,2-a]pyrimidin-3-yl]methyl]-2-azabicyclo[2.2.2]octane-4-carboxylic acid (28 mg, 90.4%). MS: m/z found 767.3 [M+H]*; ¹H NMR (400 MHz, Methanol-d₄): δ 9.20 (dd, J=7.4, 0.8 Hz, 1H), 8.60 (s, 1H), 7.87 (dd, J=1.9, 0.9 Hz, 1H), 7.76 (d, J=7.5 Hz, 1H), 7.70-7.58 (m, 4H), 7.55-7.47 (m, 2H), 7.43-7.37 (m, 1H), 7.26 (dd, J=7.4, 1.0 Hz, 1H), 4.47 (s, 2H), 4.03 (s, 3H), 3.88 (dd, J=13.2, 5.0 Hz, 3H), 3.53-3.38 (m, 3H), 2.84-2.70 (m, 2H), 2.38-2.24 (m, 4H), 2.01 (t, J=13.3 Hz, 2H), 1.93-1.78 (m, 5H).

### Example 128: ((8-(2,2′-Dichloro-3′-(6-methoxy-5-(((((S)-5-oxopyrrolidin-2-yl)methyl)amino)methyl)pyridin-2-yl)-[1,1′-biphenyl]-3-yl)-4-oxo-4H-pyrido[1,2-a]pyrimidin-3-yl)methyl)-L-proline

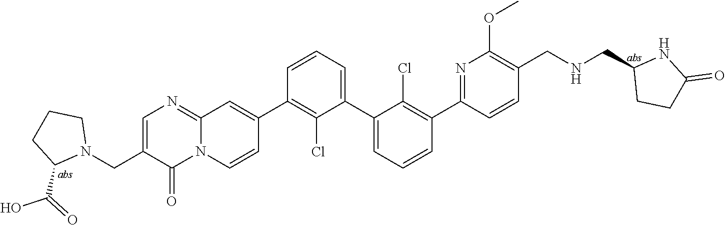 [SMILES: COc1nc(-c2cccc(-c3cccc(-c4ccn5c(=O)c(CN6CCC[C@H]6C(=O)O)cnc5c4)c3Cl)c2Cl)ccc1CNC[C@@H]1CCC(=O)N1]

### (2S)-1-[[8-[3-[3-[5-[[tert-Butoxycarbonyl-[[(2S)-5-oxopyrrolidin-2-yl]methyl]amino]methyl]-6-methoxy-2-pyridyl]-2-chloro-phenyl]-2-chloro-phenyl]-4-oxo-pyrido[1,2-a]pyrimidin-3-yl]methyl]pyrrolidine-2-carboxylic acid

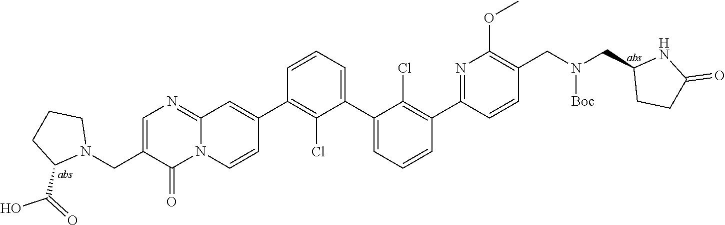 [SMILES: COc1nc(-c2cccc(-c3cccc(-c4ccn5c(=O)c(CN6CCC[C@H]6C(=O)O)cnc5c4)c3Cl)c2Cl)ccc1CN(C[C@@H]1CCC(=O)N1)C(=O)OC(C)(C)C]

A mixture of tert-butyl N-[[6-[2-chloro-3-[2-chloro-3-(3-formyl-4-oxo-pyrido[1,2-a]pyrimidin-8-yl)phenyl]phenyl]-2-methoxy-3-pyridyl]methyl]-N-[[(2S)-5-oxopyrrolidin-2-yl]methyl]carbamate (40 mg, 0.05 mmol), (2S)-pyrrolidine-2-carboxylic acid (13 mg, 0.11 mmol) and acetic acid (3 uL, 3.30 mg, 0.05 mmol) in MeOH/THF (2:1) was stirred for 30 min at rt. NaBH₃CN (7 mg, 0.11 mmol) was added, the mixture was stirred for 2 hours at rt and purified by reversed phase chromatography to provide (2S)-1-[[8-[3-[3-[5-[[tert-butoxycarbonyl-[[(2S)-5-oxopyrrolidin-2-yl]methyl]amino]methyl]-6-methoxy-2-pyridyl]-2-chloro-phenyl]-2-chloro-phenyl]-4-oxo-pyrido[1,2-a]pyrimidin-3-yl]methyl]pyrrolidine-2-carboxylic acid (35 mg, 77.0%). MS: m/z found 827.3 [M+H]⁺.

### (2S)-1-[[8-[2-chloro-3-[2-chloro-3-[6-methoxy-5-[[[(2S)-5-oxopyrrolidin-2-yl]methylamino]methyl]-2-pyridyl]phenyl]phenyl]-4-oxo-pyrido[1,2-a]pyrimidin-3-yl]methyl]pyrrolidine-2-carboxylic acid

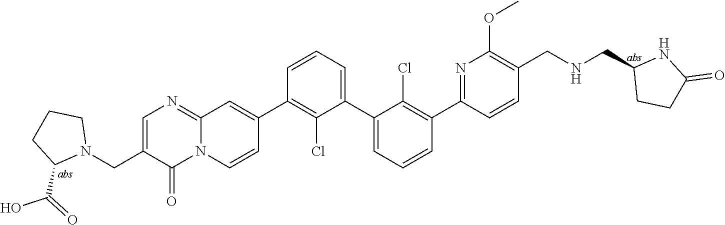 [SMILES: COc1nc(-c2cccc(-c3cccc(-c4ccn5c(=O)c(CN6CCC[C@H]6C(=O)O)cnc5c4)c3Cl)c2Cl)ccc1CNC[C@@H]1CCC(=O)N1]

A solution of (2S)-1-[[8-[3-[3-[5-[[tert-butoxycarbonyl-[[(2S)-5-oxopyrrolidin-2-yl]methyl]amino]methyl]-6-methoxy-2-pyridyl]-2-chloro-phenyl]-2-chloro-phenyl]-4-oxo-pyrido[1,2-a]pyrimidin-3-yl]methyl]pyrrolidine-2-carboxylic acid (20 mg, 0.02 mmol) in DCM/TFA (2:1) was stirred for 30 minutes, concentrated and purified by reversed phase chromatography to give (2S)-1-[[8-[2-chloro-3-[2-chloro-3-[6-methoxy-5-[[[(2S)-5-oxopyrrolidin-2-yl]methylamino]methyl]-2-pyridyl]phenyl]phenyl]-4-oxo-pyrido[1,2-a]pyrimidin-3-yl]methyl]pyrrolidine-2-carboxylic acid (16 mg, 91.0%). MS: m/z found 727.2 [M+H]+; ¹H NMR (400 MHz, Methanol-d₄): δ 9.17 (dt, J=7.4, 0.8 Hz, 1H), 8.55 (s, 1H), 7.84 (dq, J=1.8, 0.9 Hz, 1H), 7.75 (d, J=7.5 Hz, 1H), 7.68-7.64 (m, 1H), 7.64-7.57 (m, 3H), 7.53-7.47 (m, 2H), 7.39 (ddd, J=7.6, 1.8, 0.8 Hz, 1H), 7.29-7.20 (m, 1H), 4.51-4.41 (m, 2H), 4.08 (dd, J=9.4, 5.4 Hz, 1H), 4.02 (s, 3H), 3.87 (dd, J=12.4, 5.2 Hz, 3H), 3.73 (ddd, J=10.8, 6.9, 3.6 Hz, 1H), 3.36-3.32 (m, 1H), 2.82-2.71 (m, 2H), 2.54-2.42 (m, 1H), 2.38-2.25 (m, 3H), 2.16 (tdd, J=17.1, 8.2, 4.1 Hz, 2H), 1.96 (td, J=15.9, 14.5, 9.0 Hz, 1H), 1.88-1.77 (m, 1H).

### Example 129: (S)-1-((8-(2,2′-Dichloro-3′-(6-methoxy-5-(((((S)-5-oxopyrrolidin-2-yl)methyl)amino)methyl)pyridin-2-yl)-[1,1′-biphenyl]-3-yl)-4-oxo-4H-pyrido[1,2-a]pyrimidin-3-yl)methyl)piperidine-2-carboxylic acid

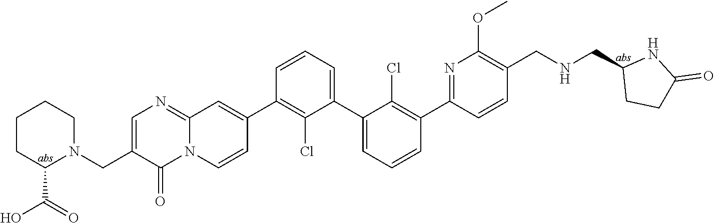 [SMILES: COc1nc(-c2cccc(-c3cccc(-c4ccn5c(=O)c(CN6CCCC[C@H]6C(=O)O)cnc5c4)c3Cl)c2Cl)ccc1CNC[C@@H]1CCC(=O)N1]

### (2S)-1-[[8-[3-[3-[5-[[tert-Butoxycarbonyl-[[(2S)-5-oxopyrrolidin-2-yl]methyl]amino]methyl]-6-methoxy-2-pyridyl]-2-chloro-phenyl]-2-chloro-phenyl]-4-oxo-pyrido[1,2-a]pyrimidin-3-yl]methyl]piperidine-2-carboxylic acid

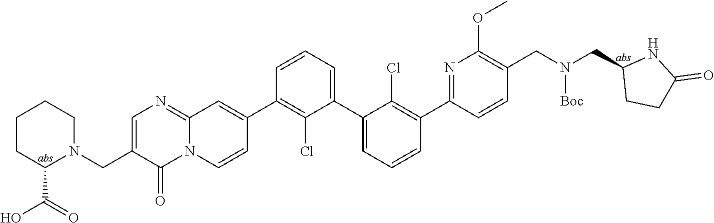 [SMILES: COc1nc(-c2cccc(-c3cccc(-c4ccn5c(=O)c(CN6CCCC[C@H]6C(=O)O)cnc5c4)c3Cl)c2Cl)ccc1CN(C[C@@H]1CCC(=O)N1)C(=O)OC(C)(C)C]

A mixture of tert-butyl N-[[6-[2-chloro-3-[2-chloro-3-(3-formyl-4-oxo-pyrido[1,2-a]pyrimidin-8-yl)phenyl]phenyl]-2-methoxy-3-pyridyl]methyl]-N-[[(2S)-5-oxopyrrolidin-2-yl]methyl]carbamate (40 mg, 0.05 mmol), (2S)-piperidine-2-carboxylic acid (14.18 mg, 0.11 mmol) and acetic acid (3 uL, 3 mg, 0.05 mmol) in MeOH/THF (2:1) was stirred for 30 min at rt. NaBH₃CN (7 mg, 0.11 mmol) was added, the mixture was stirred for 2 hours at rt and purified by reversed phase chromatography to give (2S)-1-[[8-[3-[3-[5-[[tert-butoxycarbonyl-[[(2S)-5-oxopyrrolidin-2-yl]methyl]amino]methyl]-6-methoxy-2-pyridyl]-2-chloro-phenyl]-2-chloro-phenyl]-4-oxo-pyrido[1,2-a]pyrimidin-3-yl]methyl]piperidine-2-carboxylic acid (25 mg, 54.1%). MS: m/z found 841.3 [M+H]⁺.

### (2S)-1-[[8-[2-Chloro-3-[2-chloro-3-[6-methoxy-5-[[[(2S)-5-oxopyrrolidin-2-yl]methylamino]methyl]-2-pyridyl]phenyl]phenyl]-4-oxo-pyrido[1,2-a]pyrimidin-3-yl]methyl]piperidine-2-carboxylic acid

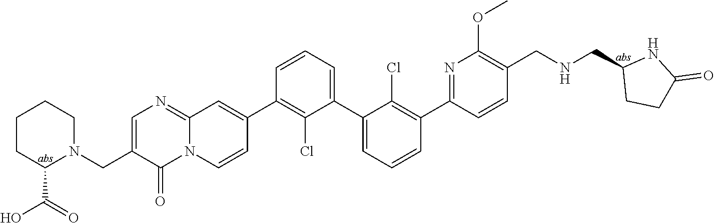 [SMILES: COc1nc(-c2cccc(-c3cccc(-c4ccn5c(=O)c(CN6CCCC[C@H]6C(=O)O)cnc5c4)c3Cl)c2Cl)ccc1CNC[C@@H]1CCC(=O)N1]

A solution of (2S)-1-[[8-[3-[3-[5-[[tert-butoxycarbonyl-[[(2S)-5-oxopyrrolidin-2-yl]methyl]amino]methyl]-6-methoxy-2-pyridyl]-2-chloro-phenyl]-2-chloro-phenyl]-4-oxo-pyrido[1,2-a]pyrimidin-3-yl]methyl]piperidine-2-carboxylic acid (25 mg, 0.03 mmol) in DCM/TFA (2:1) was stirred for 30 minutes, concentrated and purified by reversed phase chromatography to give (2S)-1-[[8-[2-chloro-3-[2-chloro-3-[6-methoxy-5-[[[(2S)-5-oxopyrrolidin-2-yl]methylamino]methyl]-2-pyridyl]phenyl]phenyl]-4-oxo-pyrido[1,2-a]pyrimidin-3-yl]methyl]piperidine-2-carboxylic acid (21 mg, 95.3%). MS: m/z found 741.2 [M+H]+; ¹H NMR (400 MHz, Methanol-d₄): δ 9.18 (dd, J=7.3, 0.7 Hz, 1H), 8.58 (d, J=1.2 Hz, 1H), 7.85 (dd, J=1.9, 0.7 Hz, 1H), 7.81 (d, J=7.5 Hz, 1H), 7.66 (dt, J=7.7, 1.8 Hz, 1H), 7.64-7.58 (m, 3H), 7.52 (ddt, J=7.7, 4.0, 2.0 Hz, 2H), 7.41 (dd, J=7.6, 1.7 Hz, 1H), 7.29 (d, J=7.5 Hz, 1H), 4.57-4.47 (m, 1H), 4.41 (d, J=13.3 Hz, 1H), 4.04 (d, J=6.9 Hz, 5H), 3.99-3.90 (m, 1H), 3.59 (t, J=6.3 Hz, 2H), 3.21-3.07 (m, 1H), 3.00-2.84 (m, 2H), 2.42-2.25 (m, 4H), 1.94-1.70 (m, 5H), 1.58 (t, J=11.9 Hz, 1H).

### Example 130: (S)-1-((8-(2,2′-Dichloro-3′-(6-methoxy-5-(((((S)-5-oxopyrrolidin-2-yl)methyl)amino)methyl)pyridin-2-yl)-[1,1′-biphenyl]-3-yl)-4-oxo-4H-pyrido[1,2-a]pyrimidin-3-yl)methyl)piperidine-3-carboxylic acid

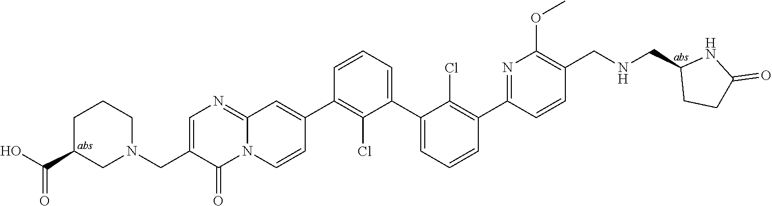 [SMILES: COc1nc(-c2cccc(-c3cccc(-c4ccn5c(=O)c(CN6CCC[C@H](C(=O)O)C6)cnc5c4)c3Cl)c2Cl)ccc1CNC[C@@H]1CCC(=O)N1]

### (3S)-1-[[8-[3-[3-[5-[[tert-Butoxycarbonyl-[[(2S)-5-oxopyrrolidin-2-yl]methyl]amino]methyl]-6-methoxy-2-pyridyl]-2-chloro-phenyl]-2-chloro-phenyl]-4-oxo-pyrido[1,2-a]pyrimidin-3-yl]methyl]piperidine-3-carboxylic acid

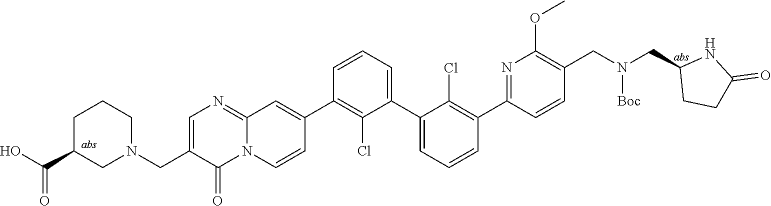 [SMILES: COc1nc(-c2cccc(-c3cccc(-c4ccn5c(=O)c(CN6CCC[C@H](C(=O)O)C6)cnc5c4)c3Cl)c2Cl)ccc1CN(C[C@@H]1CCC(=O)N1)C(=O)OC(C)(C)C]

A mixture of tert-butyl N-[[6-[2-chloro-3-[2-chloro-3-(3-formyl-4-oxo-pyrido[1,2-a]pyrimidin-8-yl)phenyl]phenyl]-2-methoxy-3-pyridyl]methyl]-N-[[(2S)-5-oxopyrrolidin-2-yl]methyl]carbamate (40 mg, 0.05 mmol), (3S)-piperidine-3-carboxylic acid (14 mg, 0.11 mmol) and acetic acid (3 uL, 3 mg, 0.05 mmol) in MeOH/THF (2:1) was stirred for 30 min at rt. NaBH₃CN (7 mg, 0.11 mmol) was added, the mixture was stirred for 2 hours at rt and purified by reversed phase chromatography to give (3S)-1-[[8-[3-[3-[5-[[tert-butoxycarbonyl-[[(2S)-5-oxopyrrolidin-2-yl]methyl]amino]methyl]-6-methoxy-2-pyridyl]-2-chloro-phenyl]-2-chloro-phenyl]-4-oxo-pyrido[1,2-a]pyrimidin-3-yl]methyl]piperidine-3-carboxylic acid (24.00 mg, 51.9%). MS: m/z found 841.3 [M+H]⁺.

### (3S)-1-[[8-[2-Chloro-3-[2-chloro-3-[6-methoxy-5-[[[(2S)-5-oxopyrrolidin-2-yl]methylamino]methyl]-2-pyridyl]phenyl]phenyl]-4-oxo-pyrido[1,2-a]pyrimidin-3-yl]methyl]piperidine-3-carboxylic acid

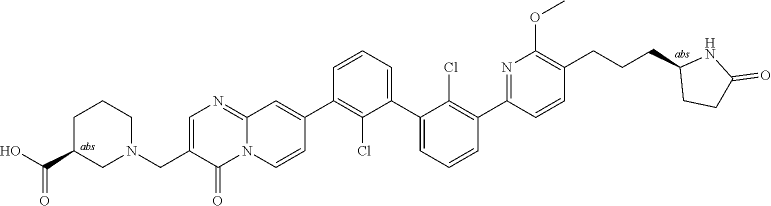 [SMILES: COc1nc(-c2cccc(-c3cccc(-c4ccn5c(=O)c(CN6CCC[C@H](C(=O)O)C6)cnc5c4)c3Cl)c2Cl)ccc1CCC[C@@H]1CCC(=O)N1]

A solution of (3S)-1-[[8-[3-[3-[5-[[tert-butoxycarbonyl-[[(2S)-5-oxopyrrolidin-2-yl]methyl]amino]methyl]-6-methoxy-2-pyridyl]-2-chloro-phenyl]-2-chloro-phenyl]-4-oxo-pyrido[1,2-a]pyrimidin-3-yl]methyl]piperidine-3-carboxylic acid (24 mg, 0.03 mmol) in DCM/TFA (2:1) was stirred for 30 minutes, concentrated and purified by reversed phase chromatography to give (3S)-1-[[8-[2-chloro-3-[2-chloro-3-[6-methoxy-5-[[[(2S)-5-oxopyrrolidin-2-yl]methylamino]methyl]-2-pyridyl]phenyl]phenyl]-4-oxo-pyrido[1,2-a]pyrimidin-3-yl]methyl]piperidine-3-carboxylic acid (20 mg, 94.6%). MS: m/z found 741.2 [M+H]⁺. ¹H NMR (400 MHz, Methanol-d₄): δ 9.17 (d, J=7.4 Hz, 1H), 8.53 (s, 1H), 8.41 (s, 1H), 7.86-7.79 (m, 2H), 7.68-7.65 (m, 1H), 7.65-7.58 (m, 3H), 7.55-7.49 (m, 2H), 7.41 (ddd, J=7.6, 1.7, 0.9 Hz, 1H), 7.30 (dd, J=7.5, 0.9 Hz, 1H), 4.37-4.25 (m, 2H), 4.08 (d, J=2.6 Hz, 2H), 4.05 (s, 3H), 3.97 (dq, J=7.6, 3.8, 2.2 Hz, 1H), 3.51-3.33 (m, 2H), 3.28-3.07 (m, 2H), 3.04-2.91 (m, 2H), 2.70 (s, 1H), 2.42-2.28 (m, 3H), 2.02-1.78 (m, 5H).

### Example 131: 8-(2,2′-dichloro-3′-(6-methoxy-5-(((((S)-5-oxopyrrolidin-2-yl)methyl)amino)methyl)pyridin-2-yl)-[1,1′-biphenyl]-3-yl)-3-((((S)-2-hydroxypropyl)amino)methyl)-4H-pyrido[1,2-a]pyrimidin-4-one

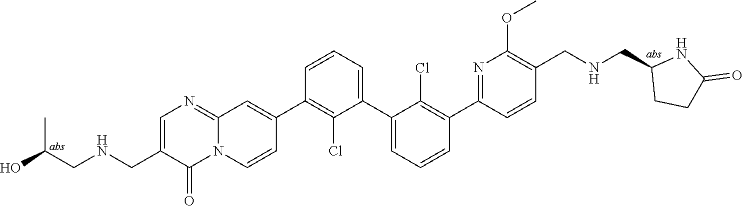 [SMILES: COc1nc(-c2cccc(-c3cccc(-c4ccn5c(=O)c(CNC[C@H](C)O)cnc5c4)c3Cl)c2Cl)ccc1CNC[C@@H]1CCC(=O)N1]

### tert-Butyl N-[[6-[2-chloro-3-[2-chloro-3-[3-[[[(2S)-2-hydroxypropyl]amino]methyl]-4-oxo-pyrido[1,2-a]pyrimidin-8-yl]phenyl]phenyl]-2-methoxy-3-pyridyl]methyl]-N-[[(2S)-5-oxopyrrolidin-2-yl]methyl]carbamate

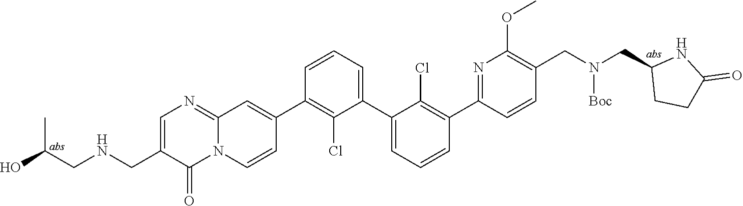 [SMILES: COc1nc(-c2cccc(-c3cccc(-c4ccn5c(=O)c(CNC[C@H](C)O)cnc5c4)c3Cl)c2Cl)ccc1CN(C[C@@H]1CCC(=O)N1)C(=O)OC(C)(C)C]

A mixture of tert-butyl N-[[6-[2-chloro-3-[2-chloro-3-(3-formyl-4-oxo-pyrido[1,2-a]pyrimidin-8-yl)phenyl]phenyl]-2-methoxy-3-pyridyl]methyl]-N-[[(2S)-5-oxopyrrolidin-2-yl]methyl]carbamate (30 mg, 0.04 mmol), (2S)-1-aminopropan-2-ol (6 mg, 0.08 mmol) and acetic acid (2. uL, 25 mg, 0.04 mmol) in MeOH/THF (2:1) was stirred for 30 min at rt. NaBH₃CN (5 mg, 0.08 mmol) was added, the mixture was stirred for 2 hours at rt, and purified by reversed phase chromatography to give tert-butyl N-[[6-[2-chloro-3-[2-chloro-3-[3-[[[(2S)-2-hydroxypropyl]amino]methyl]-4-oxo-pyrido[1,2-a]pyrimidin-8-yl]phenyl]phenyl]-2-methoxy-3-pyridyl]methyl]-N-[[(2S)-5-oxopyrrolidin-2-yl]methyl]carbamate (30 mg, 92.5%). MS: m/z found 787.3 [M+H]⁺.

### 8-[2-Chloro-3-[2-chloro-3-[6-methoxy-5-[[[(2S)-5-oxopyrrolidin-2-yl]methylamino]methyl]-2-pyridyl]phenyl]phenyl]-3-[[[(2S)-2-hydroxypropyl]amino]methyl]pyrido[1,2-a]pyrimidin-4-one

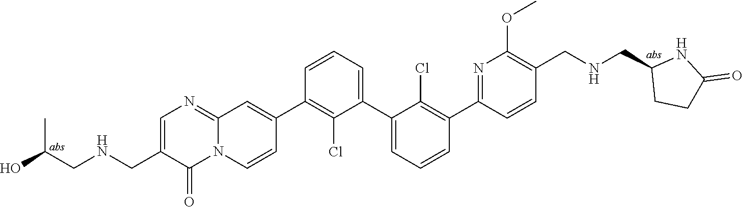 [SMILES: COc1nc(-c2cccc(-c3cccc(-c4ccn5c(=O)c(CNC[C@H](C)O)cnc5c4)c3Cl)c2Cl)ccc1CNC[C@@H]1CCC(=O)N1]

A solution of tert-butyl N-[[6-[2-chloro-3-[2-chloro-3-[3-[[[(2S)-2-hydroxypropyl]amino]methyl]-4-oxo-pyrido[1,2-a]pyrimidin-8-yl]phenyl]phenyl]-2-methoxy-3-pyridyl]methyl]-N-[[(2S)-5-oxopyrrolidin-2-yl]methyl]carbamate (30 mg, 0.04 mmol) in DCM/TFA (2:1) was stirred for 30 minutes, concentrated and purified by reversed phase chromatography to give 8-[2-chloro-3-[2-chloro-3-[6-methoxy-5-[[[(2S)-5-oxopyrrolidin-2-yl]methylamino]methyl]-2-pyridyl]phenyl]phenyl]-3-[[[(2S)-2-hydroxypropyl]amino]methyl]pyrido[1,2-a]pyrimidin-4-one (25 mg, 95.5%). MS: m/z found 687.2 [M+H]⁺. ¹H NMR (400 MHz, Methanol-d₄): δ 9.19 (dt, J=7.5, 1.0 Hz, 1H), 8.52 (s, 1H), 7.86 (dt, J=2.0, 0.9 Hz, 1H), 7.81 (dd, J=7.5, 1.2 Hz, 1H), 7.67 (dd, J=7.7, 1.7 Hz, 1H), 7.65-7.58 (m, 3H), 7.55-7.49 (m, 2H), 7.42 (dt, J=7.6, 1.5 Hz, 1H), 7.29 (dd, J=7.4, 1.2 Hz, 1H), 4.30 (s, 2H), 4.13-4.06 (m, 1H), 4.05 (s, 3H), 4.03 (d, J=3.9 Hz, 2H), 3.98-3.89 (m, 1H), 3.16 (ddd, J=12.6, 3.0, 1.2 Hz, 1H), 2.98-2.85 (m, 3H), 2.42-2.27 (m, 3H), 1.92-1.80 (m, 1H), 1.24 (d, J=6.3 Hz, 3H).

### Example 132: 8-(2,2′-Dichloro-3′-(6-methoxy-5-(((((S)-5-oxopyrrolidin-2-yl)methyl)amino)methyl)pyridin-2-yl)-[1,1′-biphenyl]-3-yl)-3-((((R)-2-hydroxypropyl)amino)methyl)-4H-pyrido[1,2-a]pyrimidin-4-one

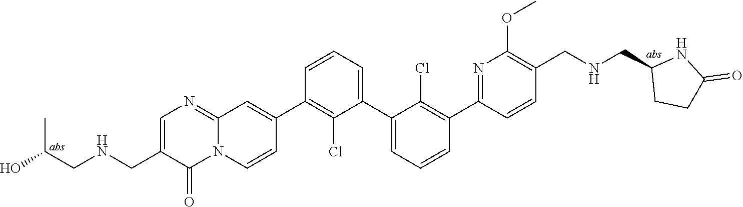 [SMILES: COc1nc(-c2cccc(-c3cccc(-c4ccn5c(=O)c(CNC[C@@H](C)O)cnc5c4)c3Cl)c2Cl)ccc1CNC[C@@H]1CCC(=O)N1]

### tert-Butyl N-[[6-[2-chloro-3-[2-chloro-3-[3-[[[(2R)-2-hydroxypropyl]amino]methyl]-4-oxo-pyrido[1,2-a]pyrimidin-8-yl]phenyl]phenyl]-2-methoxy-3-pyridyl]methyl]-N-[[(2S)-5-oxopyrrolidin-2-yl]methyl]carbamate

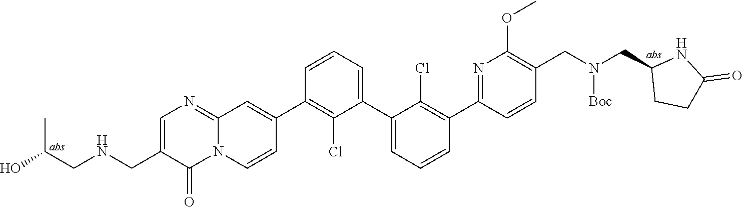 [SMILES: COc1nc(-c2cccc(-c3cccc(-c4ccn5c(=O)c(CNC[C@@H](C)O)cnc5c4)c3Cl)c2Cl)ccc1CN(C[C@@H]1CCC(=O)N1)C(=O)OC(C)(C)C]

A mixture of tert-butyl N-[[6-[2-chloro-3-[2-chloro-3-(3-formyl-4-oxo-pyrido[1,2-a]pyrimidin-8-yl)phenyl]phenyl]-2-methoxy-3-pyridyl]methyl]-N-[[(2S)-5-oxopyrrolidin-2-yl]methyl]carbamate (30 mg, 0.04 mmol), (2R)-1-aminopropan-2-ol (6 mg, 0.08 mmol) and acetic acid (2 uL, 2.5 mg, 0.04 mmol) in MeOH/THF (2:1) was stirred for 30 min at rt. NaBH₃CN (5.17 mg, 0.08 mmol) was added, the mixture was stirred for 2 hours at rt and purified by reversed phase chromatography to give tert-butyl N-[[6-[2-chloro-3-[2-chloro-3-[3-[[[(2R)-2-hydroxypropyl]amino]methyl]-4-oxo-pyrido[1,2-a]pyrimidin-8-yl]phenyl]phenyl]-2-methoxy-3-pyridyl]methyl]-N-[[(2S)-5-oxopyrrolidin-2-yl]methyl]carbamate (30 mg, 92.5%). MS: m/z found 787.3 [M+H]⁺.

### 8-[2-Chloro-3-[2-chloro-3-[6-methoxy-5-[[[(2S)-5-oxopyrrolidin-2-yl]methylamino]methyl]-2-pyridyl]phenyl]phenyl]-3-[[[(2R)-2-hydroxypropyl]amino]methyl]pyrido[1,2-a]pyrimidin-4-one

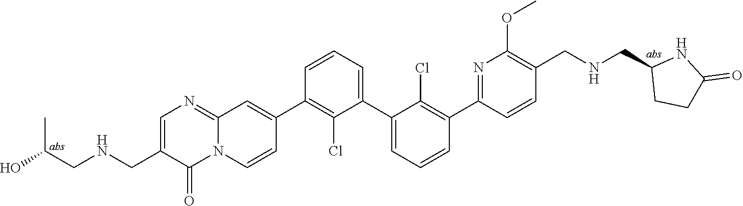 [SMILES: COc1nc(-c2cccc(-c3cccc(-c4ccn5c(=O)c(CNC[C@@H](C)O)cnc5c4)c3Cl)c2Cl)ccc1CNC[C@@H]1CCC(=O)N1]

A solution of tert-butyl N-[[6-[2-chloro-3-[2-chloro-3-[3-[[[(2R)-2-hydroxypropyl]amino]methyl]-4-oxo-pyrido[1,2-a]pyrimidin-8-yl]phenyl]phenyl]-2-methoxy-3-pyridyl]methyl]-N-[[(2S)-5-oxopyrrolidin-2-yl]methyl]carbamate (30 mg, 0.04 mmol) in DCM/TFA (2:1) was stirred for 30 minutes, concentrated and purified by reversed phase chromatography to give 8-[2-chloro-3-[2-chloro-3-[6-methoxy-5-[[[(2S)-5-oxopyrrolidin-2-yl]methylamino]methyl]-2-pyridyl]phenyl]phenyl]-3-[[[(2R)-2-hydroxypropyl]amino]methyl]pyrido[1,2-a]pyrimidin-4-one (25 mg, 95.5%). MS: m/z found 687.2 [M+H]⁺. ¹H NMR (400 MHz, Methanol-d₄): δ 9.19 (dt, J=7.4, 1.0 Hz, 1H), 8.52 (s, 1H), 7.86 (dd, J=1.9, 0.8 Hz, 1H), 7.80 (dd, J=7.5, 1.3 Hz, 1H), 7.69-7.65 (m, 1H), 7.65-7.58 (m, 3H), 7.55-7.49 (m, 2H), 7.41 (dt, J=7.6, 1.5 Hz, 1H), 7.29 (dd, J=7.4, 1.4 Hz, 1H), 4.30 (s, 2H), 4.13-4.06 (m, 1H), 4.04 (s, 3H), 4.01 (d, J=3.5 Hz, 2H), 3.95-3.89 (m, 1H), 3.16 (ddd, J=12.6, 3.0, 1.3 Hz, 1H), 2.97-2.81 (m, 3H), 2.40-2.26 (m, 3H), 1.91-1.78 (m, 1H), 1.24 (d, J=6.3 Hz, 3H).

### Example 133: (S)-8-(2,2′-Dichloro-3′-(6-methoxy-5-((((5-oxopyrrolidin-2-yl)methyl)amino)methyl)pyridin-2-yl)-[1,1′-biphenyl]-3-yl)-3-(((2-hydroxy-2-methylpropyl)amino)methyl)-4H-pyrido[1,2-a]pyrimidin-4-one

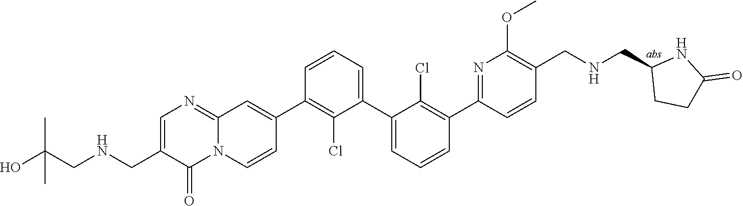 [SMILES: COc1nc(-c2cccc(-c3cccc(-c4ccn5c(=O)c(CNCC(C)(C)O)cnc5c4)c3Cl)c2Cl)ccc1CNC[C@@H]1CCC(=O)N1]

### tert-Butyl N-[[6-[2-chloro-3-[2-chloro-3-[3-[[(2-hydroxy-2-methyl-propyl)amino]methyl]-4-oxo-pyrido[1,2-a]pyrimidin-8-yl]phenyl]phenyl]-2-methoxy-3-pyridyl]methyl]-N-[[(2S)-5-oxopyrrolidin-2-yl]methyl]carbamate

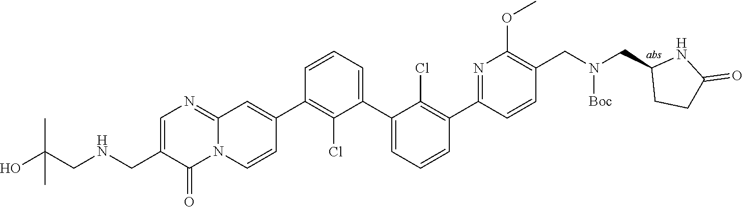 [SMILES: COc1nc(-c2cccc(-c3cccc(-c4ccn5c(=O)c(CNCC(C)(C)O)cnc5c4)c3Cl)c2Cl)ccc1CN(C[C@@H]1CCC(=O)N1)C(=O)OC(C)(C)C]

A mixture of tert-butyl N-[[6-[2-chloro-3-[2-chloro-3-(3-formyl-4-oxo-pyrido[1,2-a]pyrimidin-8-yl)phenyl]phenyl]-2-methoxy-3-pyridyl]methyl]-N-[[(2S)-5-oxopyrrolidin-2-yl]methyl]carbamate (30 mg, 0.04 mmol), 1-amino-2-methyl-propan-2-ol (7 mg, 0.08 mmol) and acetic acid (2 uL, 2.5 mg, 0.04 mmol) in MeOH/THF (2:1) was stirred for 30 min at rt. NaBH₃CN (5 mg, 0.08 mmol) was added, the mixture was stirred for 2 hours at rt and purified by reversed phase chromatography to give tert-butyl N-[[6-[2-chloro-3-[2-chloro-3-[3-[[(2-hydroxy-2-methyl-propyl)amino]methyl]-4-oxo-pyrido[1,2-a]pyrimidin-8-yl]phenyl]phenyl]-2-methoxy-3-pyridyl]methyl]-N-[[(2S)-5-oxopyrrolidin-2-yl]methyl]carbamate (29 mg, 87.8%). MS: m/z found 801.3 [M+H]⁺.

### 8-[2-Chloro-3-[2-chloro-3-[6-methoxy-5-[[[(2S)-5-oxopyrrolidin-2-yl]methylamino]methyl]-2-pyridyl]phenyl]phenyl]-3-[[(2-hydroxy-2-methyl-propyl)amino]methyl]pyrido[1,2-a]pyrimidin-4-one

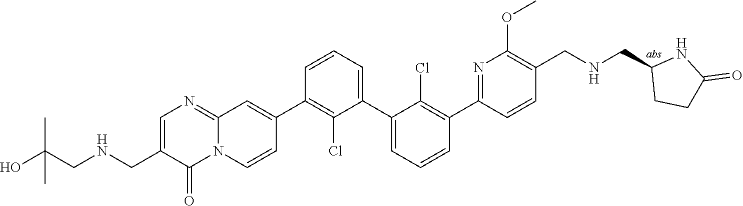 [SMILES: COc1nc(-c2cccc(-c3cccc(-c4ccn5c(=O)c(CNCC(C)(C)O)cnc5c4)c3Cl)c2Cl)ccc1CNC[C@@H]1CCC(=O)N1]

A solution of tert-butyl N-[[6-[2-chloro-3-[2-chloro-3-[3-[[(2-hydroxy-2-methyl-propyl)amino]methyl]-4-oxo-pyrido[1,2-a]pyrimidin-8-yl]phenyl]phenyl]-2-methoxy-3-pyridyl]methyl]-N-[[(2S)-5-oxopyrrolidin-2-yl]methyl]carbamate (30 mg, 0.04 mmol) in DCM/TFA (2:1) was stirred for 30 minutes, concentrated and purified by reversed phase chromatography to give 8-[2-chloro-3-[2-chloro-3-[6-methoxy-5-[[[(2S)-5-oxopyrrolidin-2-yl]methylamino]methyl]-2-pyridyl]phenyl]phenyl]-3-[[(2-hydroxy-2-methyl-propyl)amino]methyl]pyrido[1,2-a]pyrimidin-4-one (24 mg, 91.4%). MS: m/z found 701.2 [M+H]⁺. ¹H NMR (400 MHz, Methanol-d₄): δ 9.18 (dt, J=7.4, 1.0 Hz, 1H), 8.52 (s, 1H), 7.87 (dt, J=2.0, 1.0 Hz, 1H), 7.79 (dd, J=7.5, 1.3 Hz, 1H), 7.67 (dd, J=7.7, 1.7 Hz, 1H), 7.65-7.58 (m, 3H), 7.56-7.49 (m, 2H), 7.41 (dt, J=7.6, 1.6 Hz, 1H), 7.28 (dd, J=7.5, 1.3 Hz, 1H), 4.31 (s, 2H), 4.04 (s, 3H), 3.98 (d, J=3.6 Hz, 2H), 3.94-3.87 (m, 1H), 3.04 (s, 2H), 2.96-2.78 (m, 2H), 2.43-2.24 (m, 3H), 1.91-1.78 (m, 1H), 1.30 (s, 6H).

### Example 134: (2R,4R)-1-((8-(2,2′-Dichloro-3′-(6-methoxy-5-(((((S)-5-oxopyrrolidin-2-yl)methyl)amino)methyl)pyridin-2-yl)-[1,1′-biphenyl]-3-yl)-4-oxo-4H-pyrido[1,2-a]pyrimidin-3-yl)methyl)-4-hydroxypyrrolidine-2-carboxylic acid

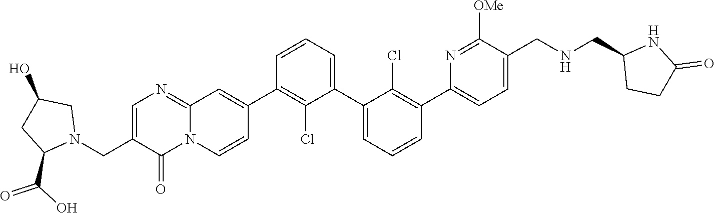 [SMILES: COc1nc(-c2cccc(-c3cccc(-c4ccn5c(=O)c(CN6C[C@H](O)C[C@@H]6C(=O)O)cnc5c4)c3Cl)c2Cl)ccc1CNC[C@@H]1CCC(=O)N1]

### (2R,4R)-1-((8-(3′-(5-(((tert-Butoxycarbonyl)(((S)-5-oxopyrrolidin-2-yl)methyl)amino)methyl)-6-methoxypyridin-2-yl)-2,2′-dichloro-[1,1′-biphenyl]-3-yl)-4-oxo-4H-pyrido[1,2-a]pyrimidin-3-yl)methyl)-4-hydroxypyrrolidine-2-carboxylic acid

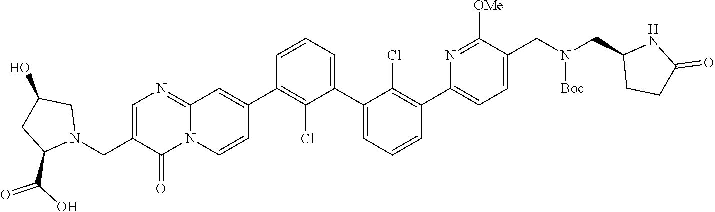 [SMILES: COc1nc(-c2cccc(-c3cccc(-c4ccn5c(=O)c(CN6C[C@H](O)C[C@@H]6C(=O)O)cnc5c4)c3Cl)c2Cl)ccc1CN(C[C@@H]1CCC(=O)N1)C(=O)OC(C)(C)C]

To a mixture of (S)-tert-butyl ((6-(2,2′-dichloro-3′-(3-formyl-4-oxo-4H-pyrido[1,2-a]pyrimidin-8-yl)-[1,1′-biphenyl]-3-yl)-2-methoxypyridin-3-yl)methyl)((5-oxopyrrolidin-2-yl)methyl)carbamate (0.25 g, 0.34 mmol) and (2R,4R)-4-hydroxypyrrolidine-2-carboxylic acid (0.09 g, 0.69 mmol) in DMF (2 mL) was added glacial acetic acid (0.1 mL, 1.72 mmol) and sodium cyanoborohydride (0.09 g, 1.37 mmol) in one portion under N₂ atmosphere. The mixture was stirred at 70° C. for 1 h. The mixture was used for next step directly without work-up. (2R,4R)-1-[[8-[3-[3-[5-[[tert-butoxycarbonyl-[[(2S)-5-oxopyrrolidin-2-yl]methyl]amino]methyl]-6-methoxy-2-pyridyl]-2-chloro-phenyl]-2-chloro-phenyl]-4-oxo-pyrido[1,2-a]pyrimidin-3-yl]methyl]-4-hydroxy-pyrrolidine-2-carboxylic acid was obtained in 2 mL of DMF solution (m/z: 843 [M+H]⁺).

### (2R,4R)-1-((8-(2,2′-Dichloro-3′-(6-methoxy-5-(((((S)-5-oxopyrrolidin-2-yl)methyl)amino)methyl)pyridin-2-yl)-[1,1′-biphenyl]-3-yl)-4-oxo-4H-pyrido[1,2-a]pyrimidin-3-yl)methyl)-4-hydroxypyrrolidine-2-carboxylic acid

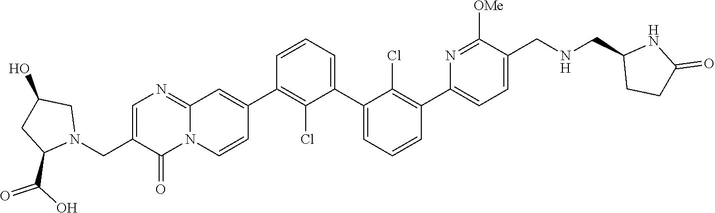 [SMILES: COc1nc(-c2cccc(-c3cccc(-c4ccn5c(=O)c(CN6C[C@H](O)C[C@@H]6C(=O)O)cnc5c4)c3Cl)c2Cl)ccc1CNC[C@@H]1CCC(=O)N1]

A mixture of (2R,4R)-1-((8-(3′-(5-(((tert-butoxycarbonyl)(((S)-5-oxopyrrolidin-2-yl)methyl)amino)methyl)-6-methoxypyridin-2-yl)-2,2′-dichloro-[1,1′-biphenyl]-3-yl)-4-oxo-4H-pyrido[1,2-a]pyrimidin-3-yl)methyl)-4-hydroxypyrrolidine-2-carboxylic acid in 2 mL of DMF solution and trifluoroacetic acid (6 mL, 81 mmol) was stirred at r.t. for 2 h. The mixture was concentrated. The residue was purified by reverse phase HPLC to afford (2R,4R)-1-((8-(2,2′-dichloro-3′-(6-methoxy-5-(((((S)-5-oxopyrrolidin-2-yl)methyl)amino)methyl)pyridin-2-yl)-[1,1′-biphenyl]-3-yl)-4-oxo-4H-pyrido[1,2-a]pyrimidin-3-yl)methyl)-4-hydroxypyrrolidine-2-carboxylic acid as a white solid (58.8 mg, 23% yield for two steps, m/z: 743 [M+H]⁺). ¹H NMR (400 MHz, Methanol-d₄) δ 9.18 (d, J=7.2 Hz, 1H), 8.56 (s, 1H), 7.84-7.82 (m, 2H), 7.69-7.59 (m, 4H), 7.55-7.51 (m, 2H), 7.43-7.41 (m, 1H), 7.31 (d, J=7.2 Hz, 1H), 4.50 (s, 3H), 4.20-4.06 (m, 6H), 3.96 (m, 1H), 3.68-3.65 (m, 1H), 3.41-3.39 (m, 1H), 2.97-2.94 (m, 2H), 2.72-2.64 (m, 1H), 2.43-2.28 (m, 4H), 1.93-1.84 (m, 1H).

### Example 135: (S)-1-((6-(2,2′-Dichloro-3′-(3-((3-(hydroxymethyl)azetidin-1-yl)methyl)-4-oxo-4H-pyrido[1,2-a]pyrimidin-8-yl)-[1,1′-biphenyl]-3-yl)-2-methoxypyridin-3-yl)methyl)pyrrolidine-3-carboxylic acid

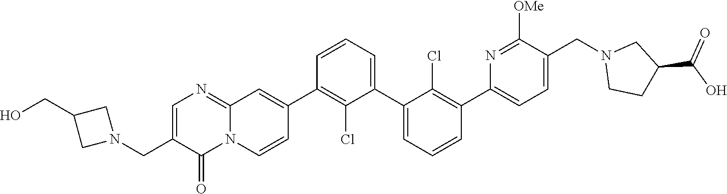 [SMILES: COc1nc(-c2cccc(-c3cccc(-c4ccn5c(=O)c(CN6CC(CO)C6)cnc5c4)c3Cl)c2Cl)ccc1CN1CC[C@H](C(=O)O)C1]

### 8-(3-Bromo-2-chlorophenyl)-3-((3-(hydroxymethyl)azetidin-1-yl)methyl)-4H-pyrido[1,2-a]pyrimidin-4-one

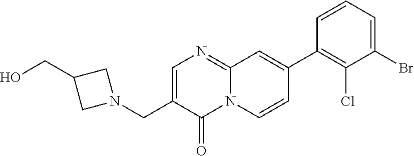 [SMILES: O=c1c(CN2CC(CO)C2)cnc2cc(-c3cccc(Br)c3Cl)ccn12]

To a mixture of 8-(3-bromo-2-chlorophenyl)-4-oxo-4H-pyrido[1,2-a]pyrimidine-3-carbaldehyde (1.5 g, 4.13 mmol) and azetidin-3-ylmethanoll as a hydrochloride salt (0.76 g, 6.19 mmol) in DCM (15 mL) was added sodium acetate (0.68 g, 8.25 mmol) and sodium triacetoxyborohydride (2.62 g, 12.4 mmol), then the mixture was stirred at r.t. for 3 hr under N₂. The mixture was filtered and concentrated. The residue was purified by reverse phase HPLC to afford 8-(3-bromo-2-chlorophenyl)-3-((3-(hydroxymethyl)azetidin-1-yl)methyl)-4H-pyrido[1,2-a]pyrimidin-4-one as a brown solid (1.5 g).

### 6-(2,2′-Dichloro-3′-(3-((3-(hydroxymethyl)azetidin-1-yl)methyl)-4-oxo-4H-pyrido[1,2-a]pyrimidin-8-yl)-[1,1′-biphenyl]-3-yl)-2-methoxynicotinaldehyde

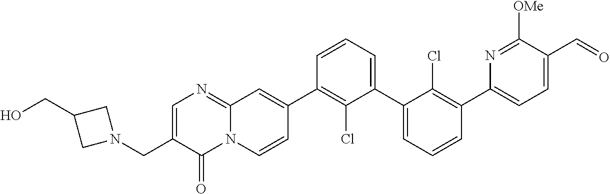 [SMILES: COc1nc(-c2cccc(-c3cccc(-c4ccn5c(=O)c(CN6CC(CO)C6)cnc5c4)c3Cl)c2Cl)ccc1C=O]

To a mixture 8-(3-bromo-2-chlorophenyl)-3-((3-(hydroxymethyl)azetidin-1-yl)methyl)-4H-pyrido[1,2-a]pyrimidin-4-one (0.3 g, 0.69 mmol) in dioxane/H₂O (4:1, 2.5 mL) and (0.5 mL) was added 6-(2-chloro-3-(4,4,5,5-tetramethyl-1,3,2-dioxaborolan-2-yl)phenyl)-2-methoxynicotinaldehyde (0.35 g, 0.68 mmol), [1, 1′-bis(di-tert-butylphosphino)ferrocene]dichloropalladium(II) (0.04 g, 0.07 mmol) and potassium phosphate (0.43 g, 2.07 mmol). After that, the mixture was stirred at 110° C. for 0.5 hr. The mixture was combined with another batch at 0.1 g scale. The reaction mixture was added to water (30 mL), and then extracted with EtOAc (3×30 ml). The combined organic layers were washed with saturated aqueous brine solution (20 mL), dried over anhydrous sodium sulfate, filtered and concentrated under reduced pressure to give a residue. The residue was purified by normal phase SiO₂ chromatography (0-100% EtOAc/petroleum ether) to afford 6-(2,2′-dichloro-3′-(3-((3-(hydroxymethyl)azetidin-1-yl)methyl)-4-oxo-4H-pyrido[1,2-a]pyrimidin-8-yl)-[1,1′-biphenyl]-3-yl)-2-methoxynicotinaldehyde as a yellow solid (0.26 g). MS: m/z found 601 [M+H]⁺.

### (S)-1-((6-(2,2′-Dichloro-3′-(3-((3-(hydroxymethyl)azetidin-1-yl)methyl)-4-oxo-4H-pyrido[1,2-a]pyrimidin-8-yl)-[1,1′-biphenyl]-3-yl)-2-methoxypyridin-3-yl)methyl)pyrrolidine-3-carboxylic acid

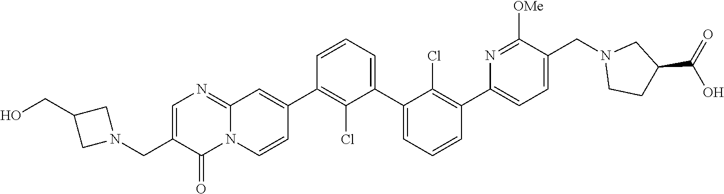 [SMILES: COc1nc(-c2cccc(-c3cccc(-c4ccn5c(=O)c(CN6CC(CO)C6)cnc5c4)c3Cl)c2Cl)ccc1CN1CC[C@H](C(=O)O)C1]

To a mixture 6-(2,2′-dichloro-3′-(3-((3-(hydroxymethyl)azetidin-1-yl)methyl)-4-oxo-4H-pyrido[1,2-a]pyrimidin-8-yl)-[1,1′-biphenyl]-3-yl)-2-methoxynicotinaldehyde (0.12 g, 0.2 mmol) in DCM (3 mL) was added (S)-pyrrolidine-3-carboxylic acid (0.07 g, 0.6 mmol) and sodium acetate (0.05 g, 0.6 mmol). The resulting mixture was stirred at r.t. for 10 hr. To the mixture was added sodium triacetoxyborohydride (0.13 g, 0.6 mmol) and the reaction mixture was stirred at r.t. for 2 hrs. The reaction mixture was filtered and concentrated under reduced pressure to give a residue. The residue was purified by reverse phase HPLC to afford (S)-1-((6-(2,2′-dichloro-3′-(3-((3-(hydroxymethyl)azetidin-1-yl)methyl)-4-oxo-4H-pyrido[1,2-a]pyrimidin-8-yl)-[1,1′-biphenyl]-3-yl)-2-methoxypyridin-3-yl)methyl)pyrrolidine-3-carboxylic acid as a white solid (36.4 mg, 25% yield, MS: 700.1 [M+H]⁺ observed). ¹H NMR (400 MHz, Methanol-d₄): δ 9.12 (d, J=7.2 Hz, 1H), 8.38 (s, 1H), 8.87 (d, J=7.6 Hz, 1H), 7.78 (d, J=0.8 Hz, 1H), 7.66 (dd, J=1.2 Hz, J=6 Hz, 1H), 7.62-7.54 (m, 2H), 7.52-7.540 (m, 3H), 7.41 (dd, J=1.2 Hz, J=6 Hz, 1H), 7.32 (d, J=7.6 Hz, 1H), 4.21 (s, 2H), 4.06 (s, 3H), 3.91 (s, 2H), 3.72 (t, J=8.8 Hz, 2H), 3.65 (d, J=6 Hz, 2H), 3.46 (t, J=8 Hz, 2H), 3.34-3.32 (m, 2H), 3.20 (t, J=7.2 Hz, 2H), 3.09-3.03 (m, 1H), 2.80-2.74 (m, 1H), 2.30-2.18 (m, 2H).

### Example 136: 1-((6-(2,2′-Dichloro-3′-(3-((3-(hydroxymethyl)azetidin-1-yl)methyl)-4-oxo-4H-pyrido[1,2-a]pyrimidin-8-yl)-[1,1′-biphenyl]-3-yl)-2-methoxypyridin-3-yl)methyl)azetidine-3-carboxylic acid

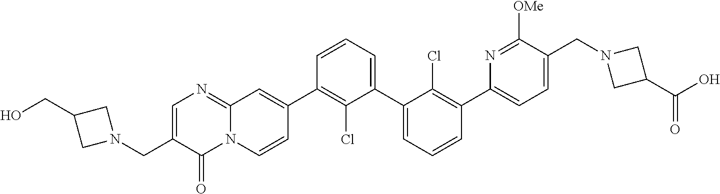 [SMILES: COc1nc(-c2cccc(-c3cccc(-c4ccn5c(=O)c(CN6CC(CO)C6)cnc5c4)c3Cl)c2Cl)ccc1CN1CC(C(=O)O)C1]

To a mixture 6-(2,2′-dichloro-3′-(3-((3-(hydroxymethyl)azetidin-1-yl)methyl)-4-oxo-4H-pyrido[1,2-a]pyrimidin-8-yl)-[1,1′-biphenyl]-3-yl)-2-methoxynicotinaldehyde (0.12 g, 0.2 mmol) in DCM (3 mL) was added azetidine-3-carboxylic acid (60.5 mg, 0.6 mmol) and sodium acetate (0.05 g, 0.6 mmol). After that, the mixture was stirred at r.t. for 10 hr. To the mixture was added sodium triacetoxyborohydride (0.13 g, 0.6 mmol). After that, the mixture was stirred at r.t. for 2 hr. The reaction mixture was filtered and concentrated under reduced pressure to give a residue. The residue was purified by reverse phase HPLC to afford 1-((6-(2,2′-dichloro-3′-(3-((3-(hydroxymethyl)azetidin-1-yl)methyl)-4-oxo-4H-pyrido[1,2-a]pyrimidin-8-yl)-[1,1′-biphenyl]-3-yl)-2-methoxypyridin-3-yl)methyl)azetidine-3-carboxylic acid as a white solid. (22.2 mg, 16.04% yield, MS: 686.1 [M+H]+ observed). ¹H NMR (400 MHz, Methanol-d₄): 9.13 (d, J=7.6 Hz, 1H), 8.40 (s, 1H), 7.80-7.78 (m, 2H), 7.67-7.65 (m, 1H), 7.63-7.59 (m, 2H), 7.55 (dd, J=2 Hz, J=7.2 Hz, 1H), 7.53-7.49 (m, 2H), 7.40 (dd, J=1.2 Hz, J=8 Hz, 1H), 7.29 (d, J=7.6 Hz, 1H), 4.12 (s, 2H), 4.03 (s, 3H), 4.02-3.97 (m, 4H), 3.90 (t, J=8 Hz, 2H), 3.80 (t, J=8.4 Hz, 2H), 3.66 (d, J=5.6 Hz, 2H), 3.57 (t, J=8.8 Hz, 2H), 3.39-330 (m, 1H), 2.83-2.80 (m, 1H).

### Example 137: Methyl (S)-4-(((8-(2,2′-dichloro-3′-(6-methoxy-5-((((5-oxopyrrolidin-2-yl)methyl)amino)methyl)pyridin-2-yl)-[1,1′-biphenyl]-3-yl)-4-oxo-4H-pyrido[1,2-a]pyrimidin-3-yl)methyl)amino)butanoate

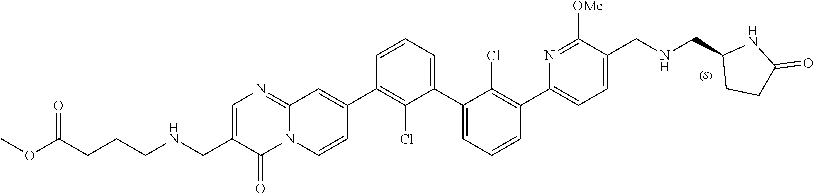

### (S)-4-(((8-(3′-(5-(((tert-Butoxycarbonyl)((5-oxopyrrolidin-2-yl)methyl)amino)methyl)-6-Methoxypyridin-2-yl)-2,2′-dichloro-[1,1′-biphenyl]-3-yl)-4-oxo-4H-pyrido[1,2-a]pyrimidin-3-yl)methyl)amino)butanoic acid

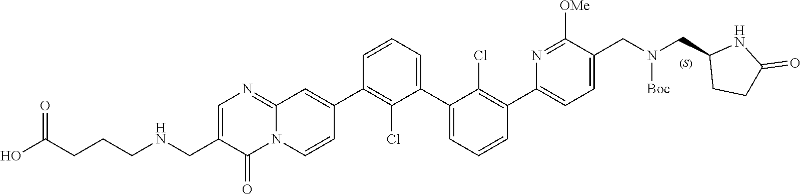

To a stirring solution of tert-butyl N-[[6-[2-chloro-3-[2-chloro-3-(3-formyl-4-oxo-pyrido[1,2-a]pyrimidin-8-yl)phenyl]phenyl]-2-methoxy-3-pyridyl]methyl]-N-[[rac-(2S)-5-oxopyrrolidin-2-yl]methyl]carbamate (45 mg, 0.06 mmol) in MeOH (1 mL) and THE (0.2 mL) at rt was added acetic acid (4 mg, 0.06 mmol), followed by 4-aminobutanoic acid (30 □L, 0.31 mmol) in one portion, followed by activated 4 AMS (15 mg). After 0.5 h of stirring at rt, sodium triacetoxyborohydride (26 mg, 0.12 mmol) was added in one portion. After an additional 0.5 h stirring, the MS were filtered, and the filtrate was evaporated to dryness. The crude (S)-4-(((8-(3′-(5-(((tert-butoxycarbonyl)((5-oxopyrrolidin-2-yl)methyl)amino)methyl)-6-methoxypyridin-2-yl)-2,2′-dichloro-[1,1′-biphenyl]-3-yl)-4-oxo-4H-pyrido[1,2-a]pyrimidin-3-yl)methyl)amino)butanoic acid as a red oil was used in the next reaction without further purification. MS: m/z found 817.3 [M+H]⁺.

### Methyl (S)-4-(((8-(2,2′-dichloro-3′-(6-methoxy-5-((((5-oxopyrrolidin-2-yl)methyl)amino)methyl)pyridin-2-yl)-[1,1′-biphenyl]-3-yl)-4-oxo-4H-pyrido[1,2-a]pyrimidin-3-yl)methyl)amino)butanoate

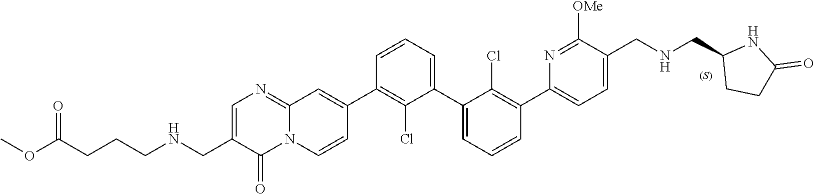

To a stirring solution of crude 4-[[8-[3-[3-[5-[[tert-butoxycarbonyl-[[rac-(2S)-5-oxopyrrolidin-2-yl]methyl]amino]methyl]-6-methoxy-2-pyridyl]-2-chloro-phenyl]-2-chloro-phenyl]-4-oxo-pyrido[1,2-a]pyrimidin-3-yl]methylamino]butanoic acid (60 mg, 0.07 mmol) was treated with a 4 N solution of hydrogen chloride (0.18 mL, 0.74 mmol) in dioxane at rt. After 6 h the solvent and excess acid were removed under vacuum. The crude material was dissolved in DMSO (2 mL) and purified by HPLC from 10-75% MeCN/H₂O over a ten-minute ramp to afford methyl (S)-4-(((8-(2,2′-dichloro-3′-(6-methoxy-5-((((5-oxopyrrolidin-2yl)methyl)amino)methyl)pyridin-2-yl)-[1,1′-biphenyl]-3-yl)-4-oxo-4H-pyrido[1,2-a]pyrimidin-3-yl)methyl)amino)butanoate (29.5 mg) as a white solid. MS: m/z found 729.3 [M+H]⁺. ¹H NMR (400 MHz, DMSO-d₆) δ 9.01 (d, J=7.4 Hz, 1H), 8.39 (s, 1H), 8.22 (s, 2H), 7.82 (d, J=7.5 Hz, 1H), 7.77 (d, J=1.9 Hz, 1H), 7.72-7.64 (m, 3H), 7.65-7.59 (m, 1H), 7.58-7.53 (m, 2H), 7.50-7.44 (m, 2H), 7.27 (dd, J=7.5, 1.3 Hz, 1H), 3.91 (d, J=1.3 Hz, 3H), 3.71 (d, J=4.2 Hz, 4H), 3.64-3.60 (m, 1H), 3.56 (d, J=1.3 Hz, 3H), 2.59-2.51 (m, 5H), 2.35 (t, J=7.3 Hz, 2H), 2.15-2.05 (m, 3H), 1.69 (t, J=7.2 Hz, 2H).

### Example 138: (S)-8-(2,2′-Dichloro-3′-(6-methoxy-5-((((5-oxopyrrolidin-2-yl)methyl)amino)methyl)pyridin-2-yl)-[1,1′-biphenyl]-3-yl)-3-(((2,4-dimethoxybenzyl)amino)methyl)-4H-pyrido[1,2-a]pyrimidin-4-one

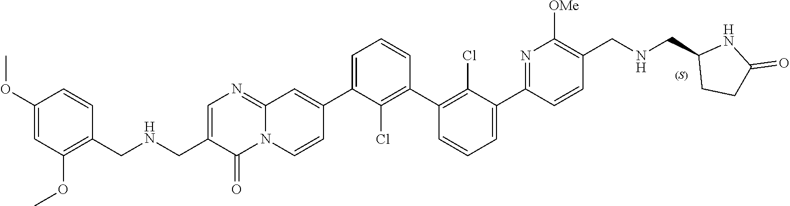

### tert-Butyl (S)-((6-(2,2′-dichloro-3′-(3-(((2,4-dimethoxybenzyl)amino)methyl)-4-oxo-4H-pyrido[1,2-a]pyrimidin-8-yl)-[1,1′-biphenyl]-3-yl)-2-methoxypyridin-3-yl)methyl)((5-oxopyrrolidin-2-yl)methyl)carbamate

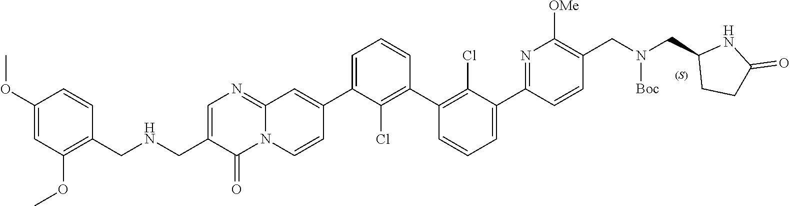

To a stirring solution of tert-butyl N-[[6-[2-chloro-3-[2-chloro-3-(3-formyl-4-oxo-pyrido[1,2-a]pyrimidin-8-yl)phenyl]phenyl]-2-methoxy-3-pyridyl]methyl]-N-[[rac-(2S)-5-oxopyrrolidin-2-yl]methyl]carbamate (45.00 mg, 0.06 mmol) in MeOH (1 mL) and THE (0.2 mL) at rt was added acetic acid (4 mg, 0.06 mmol) and (2,4-dimethoxyphenyl)methanamine (20 □L, 0.12 mmol). After 1 h of stirring at rt, sodium cyanoborohydride (8 mg, 0.12 mmol) was added in one portion. After 30 min additional stirring, the solvent was removed under reduced pressure to afford tert-butyl (S)-((6-(2,2′-dichloro-3′-(3-(((2,4-dimethoxybenzyl)amino)methyl)-4-oxo-4H-pyrido[1,2-a]pyrimidin-8-yl)-[1,1′-biphenyl]-3-yl)-2-methoxypyridin-3-yl)methyl)((5-oxopyrrolidin-2-yl)methyl)carbamate crude as a red oil that was used in the next reaction without further purification. MS: m/z found 879.4[M+H]⁺.

### (S)-8-(2,2′-Dichloro-3′-(6-methoxy-5-((((5-oxopyrrolidin-2-yl)methyl)amino)methyl)pyridin-2-yl)-[1,1′-biphenyl]-3-yl)-3-(((2,4-dimethoxybenzyl)amino)methyl)-4H-pyrido[1,2-a]pyrimidin-4-one

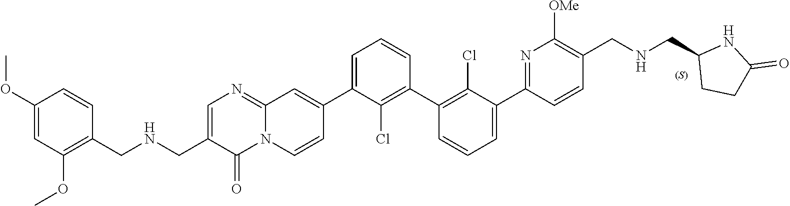

To a stirring solution of crude tert-butyl N-[[6-[2-chloro-3-[2-chloro-3-[3-[[(2,4-dimethoxyphenyl)methylamino]methyl]-4-oxo-pyrido[1,2-a]pyrimidin-8-yl]phenyl]phenyl]-2-methoxy-3-pyridyl]methyl]-N-[[rac-(2S)-5-oxopyrrolidin-2-yl]methyl]carbamate (45 mg, 0.05 mmol) in DCM (1 mL) at rt was added trifluoroacetic acid (0.20 mL, 2.56 mmol) in a steady stream. The reaction was stirred for 3 h, the solvent and excess acid were removed under reduced pressure and concentrated to dryness. The resulting crude mixture was purified by normal phase silica gel column chromatography from 0-25% MeOH/CH₂Cl₂ to afford (S)-8-(2,2′-dichloro-3′-(6-methoxy-5-((((5-oxopyrrolidin-2-yl)methyl)amino)methyl)pyridin-2-yl)-[1,1′-biphenyl]-3-yl)-3-(((2,4-dimethoxybenzyl)amino)methyl)-4H-pyrido[1,2-a]pyrimidin-4-one (38 mg) as a white solid. MS: m/z found 781.4 [M+H]⁺. ¹H NMR (400 MHz, DMSO-d₆) δ 9.08 (d, J=7.4 Hz, 1H), 8.80 (s, 1H), 8.51 (s, 1H), 7.96 (d, J=7.6 Hz, 1H), 7.88 (d, J=1.9 Hz, 1H), 7.71-7.63 (m, 3H), 7.63-7.56 (m, 4H), 7.51 (dd, J=7.5, 1.7 Hz, 1H), 7.39 (d, J=7.5 Hz, 1H), 7.30 (d, J=8.4 Hz, 1H), 6.60 (d, J=2.4 Hz, 1H), 6.53 (dd, J=8.4, 2.3 Hz, 1H), 4.21 (s, 2H), 4.14 (s, 2H), 4.09 (s, 2H), 3.97 (s, 3H), 3.89 (d, J=6.2 Hz, 1H), 3.81 (s, 3H), 3.75 (s, 3H), 3.15 (s, 2H), 3.08 (d, J=7.4 Hz, 1H), 2.18 (td, J=12.9, 12.4, 8.4 Hz, 3H), 1.79 (d, J=6.6 Hz, 1H).

### Example 139: (S)-2-(1-(((8-(2,2′-Dichloro-3′-(6-methoxy-5-((((5-oxopyrrolidin-2-yl)methyl)amino)methyl)-pyridin-2-yl)-[1,1′-biphenyl]-3-yl)-4-oxo-4H-pyrido[1,2-a]pyrimidin-3-yl)methyl)amino)-cyclopropyl)acetic acid

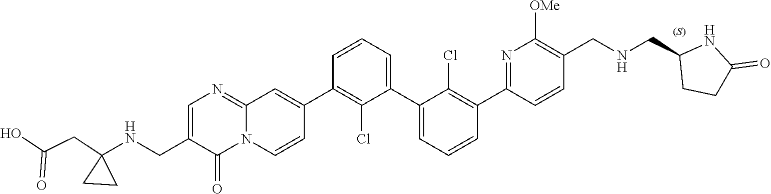

To a solution of tert-butyl N-[[6-[2-chloro-3-[2-chloro-3-(3-formyl-4-oxo-pyrido[1,2-a]-pyrimidin-8-yl)phenyl]phenyl]-2-methoxy-3-pyridyl]methyl]-N-[[(2S)-5-oxopyrrolidin-2-yl]-methyl]carbamate (50 mg, 0.07 mmol) and 2-(1-aminocyclopropyl)acetic acid (45 mg, 0.26 mmol) in dioxane (5 mL) and DMSO (1 mL), acetic acid (20 mg, 0.32 mmol) was added and stirred at 60° C. for 2 hrs and then added sodium triacetoxyborohydride (150 mg, 0.7 mmol) and stirred for 1 h at room temperature. Reaction mixture was diluted with water (20 mL), precipitate filtered, washed with water (10 mL) to give (S)-2-(1-(((8-(3′-(5-(((tert-butoxycarbonyl)((5-oxopyrrolidin-2-yl)methyl)amino)methyl)-6-methoxypyrazin-2-yl)-2,2′-dichloro-[1,1′-biphenyl]-3-yl)-4-oxo-4H-pyrido[1,2-a]pyrimidin-3-yl)methyl)amino)cyclopropyl)acetic acid as an orange solid and used for next reaction without further purification (42 mg, 71.4% yield), m/z: 828 [M+H]⁺ observed.

To the (S)-2-(1-(((8-(3′-(5-(((tert-butoxycarbonyl)((5-oxopyrrolidin-2-yl)methyl)amino)methyl)-6-methoxypyrazin-2-yl)-2,2′-dichloro-[1,1′-biphenyl]-3-yl)-4-oxo-4H-pyrido[1,2-a]pyrimidin-3-yl)methyl)amino)cyclopropyl)acetic acid (42 mg, 0.051) in dioxane (5 mL), HCl in dioxane (1.5 mL, 6 mmol, 4M HCl in dioxane) was added and vigorously stirred for 2 h and then neutralized the solution with aqueous HCl solution. Solvents evaporated and crude product was purified by HPLC with eluent 0 to 100% acetonitrile in water (formic acid as modifier) furnished (S)-2-(1-(((8-(2,2′-dichloro-3′-(6-methoxy-5-((((5-oxopyrrolidin-2-yl)methyl)amino)-methyl)-pyridin-2-yl)-[1,1′-biphenyl]-3-yl)-4-oxo-4H-pyrido[1,2-a]pyrimidin-3-yl)methyl)-amino)-cyclopropyl)-acetic acid (as bis-formic acid salt) as a white solid (15 mg, 29.5% yield). m/z found: 727 [M+H]+ observed. ¹H NMR (400 MHz, Methanol-d₄) δ 9.05 (d, J=7.2 Hz, 1H), 8.48 (s, 1H), 8.44 (s, 1H), 7.82 (d, J=7.2 Hz, 1H), 7.73 (s, 1H), 7.63 (d, J=7.2 Hz, 1H), 7.56-7.45 (m, 4H), 7.35 (dd, J=7.2 Hz, J=1.6 Hz, 1H), 7.27 (d, J=7.2 Hz, 1H), 4.27 (s, 2H), 4.08-3.90 (m, 5H), 3.31-3.29 (m, 3H), 3.05-2.93 (m, 2H), 2.58 (s, 2H), 2.40-2.31 (m, 3H), 1.90-1.82 (m, 1H), 1.2 (s, 2H), 0.83 (s, 2H).

### Example 140: 8-(2,2′-Dichloro-5-fluoro-3′-(6-methoxy-5-(((((S)-5-oxopyrrolidin-2-yl)methyl)amino)methyl)pyridin-2-yl)-[1,1′-biphenyl]-3-yl)-3-(((((S)-5-oxopyrrolidin-2-yl)methyl)amino)methyl)-4H-pyrido[1,2-a]pyrimidin-4-one

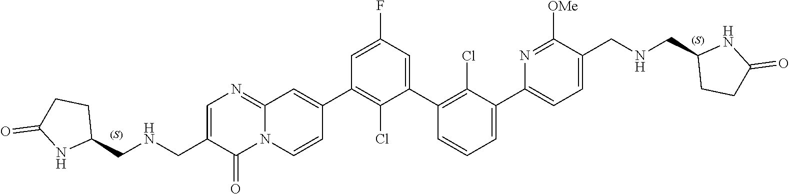

### 6-(3′-Bromo-2,2′-dichloro-5′-fluoro-[1,1′-biphenyl]-3-yl)-2-methoxynicotinaldehyde

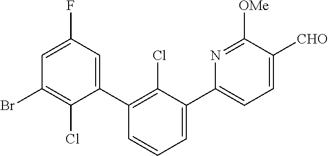 [SMILES: COc1nc(-c2cccc(-c3cc(F)cc(Br)c3Cl)c2Cl)ccc1C=O]

To a solution of 6-(2-chloro-3-(4,4,5,5-tetramethyl-1,3,2-dioxaborolan-2-yl)phenyl)-2-methoxynicotinaldehyde (0.15 g, 0.4 mmol) and 1,3-dibromo-2-chloro-5-fluorobenzene (0.17 g, 0.6 mmol) in dioxane (10 mL) under nitrogen gas was added 1,1′-bis(diphenylphosphino)ferrocene-palladium(ii) dichloride (DCM adduct) (0.03 g, 0.04 mmol) and potassium carbonate (0.14 g, 1.0 mmol). The mixture was stirred at 90° C. for 3 hrs. The mixture was filtered through CELITE® and the filter cake was washed with EtOAc (3×40 mL). The filtrate was washed with saturated aqueous brine solution (2×30 mL), then dried over Na₂SO₄ and concentrated under reduced pressure. The residue was purified by normal phase flash SiO₂ chromatography using a linear gradient (0-40% ethyl acetate hexanes) to afford 6-(3′-bromo-2,2′-dichloro-5′-fluoro-[1,1′-biphenyl]-3-yl)-2-methoxynicotinaldehyde (0.066 g, 87% yield). MS: m/z found 453 [M+H]⁺.

### 8-(2,2′-Dichloro-5-fluoro-3′-(5-formyl-6-methoxypyridin-2-yl)-[1,1′-biphenyl]-3-yl)-4-oxo-4H-pyrido[1,2-a]pyrimidine-3-carbaldehyde

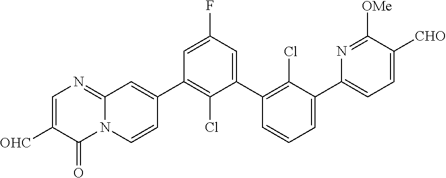 [SMILES: COc1nc(-c2cccc(-c3cc(F)cc(-c4ccn5c(=O)c(C=O)cnc5c4)c3Cl)c2Cl)ccc1C=O]

To a solution of 6-(3′-bromo-2,2′-dichloro-5′-fluoro-[1,1′-biphenyl]-3-yl)-2-methoxynicotinaldehyde (0.09 g, 0.20 mmol) and (3-formyl-4-oxo-4H-pyrido[1,2-a]pyrimidin-8-yl)boronic acid (0.06 g, 0.26 mmol) in dioxane (10 mL) under nitrogen gas was added 1,1′-bis(diphenylphosphino)ferrocene-palladium(ii) dichloride (DCM adduct) (0.01 g, 0.01 mmol) and potassium carbonate (0.07 g, 0.49 mmol). The mixture was stirred at 90° C. for 3 hrs. The mixture was filtered through CELITE® and the filter cake was washed with EtOAc (3×40 mL). The filtrate was washed with saturated aqueous brine solution (2×30 mL), then dried over Na₂SO₄ and concentrated under reduced pressure. The residue was purified by normal phase flash SiO₂ chromatography using a linear gradient (0-3% MeOH/DCM) to afford 8-(2,2′-dichloro-5-fluoro-3′-(5-formyl-6-methoxypyridin-2-yl)-[1,1′-biphenyl]-3-yl)-4-oxo-4H-pyrido[1,2-a]pyrimidine-3-carbaldehyde (0.032 g, 31% yield). MS m/z found 548 [M+H]⁺.

### 8-(2,2′-Dichloro-5-fluoro-3′-(6-methoxy-5-(((((S)-5-oxopyrrolidin-2-yl)methyl)amino)methyl)pyridin-2-yl)-[1,1′-biphenyl]-3-yl)-3-(((((S)-5-oxopyrrolidin-2-yl)methyl)amino)methyl)-4H-pyrido[1,2-a]pyrimidin-4-one

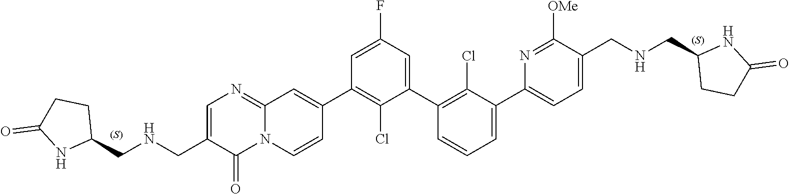

To a mixture of 8-(2,2′-dichloro-5-fluoro-3′-(5-formyl-6-methoxypyridin-2-yl)-[1,1′-biphenyl]-3-yl)-4-oxo-4H-pyrido[1,2-a]pyrimidine-3-carbaldehyde (0.03 g, 0.06 mmol) and (S)-5-(aminomethyl)pyrrolidin-2-one (0.02 g, 0.20 mmol) in 1:1 THF/MeOH (6 mL) was added acetic acid (0.01 g, 0.18 mmol) and 4 Å molecular sieve (1 g). The mixture was stirred at r.t. for 3 hrs. Sodium cyanoborohydride (0.01 g, 0.20 mmol) was added and the mixture was stirred at r.t. for 1 hr. The reaction was concentrated, and residue purified by Gilson (with 0.05% Formic acid modifier). ACN was evaporated off, and aqueous portion was lyophilized to give 15.4 mg (36%) of the product as bis formic acid salt affording 8-(2,2′-dichloro-5-fluoro-3′-(6-methoxy-5-(((((S)-5-oxopyrrolidin-2-yl)methyl)amino)methyl)pyridin-2-yl)-[1,1′-biphenyl]-3-yl)-3-(((((S)-5-oxopyrrolidin-2-yl)methyl)amino)methyl)-4H-pyrido[1,2-a]pyrimidin-4-one as a white solid. MS: m/z found 744 [M+H]⁺. ¹H NMR (400 MHz, Methanol-d₄) δ 9.16 (d, J=7.5, 0.7 Hz, 1H), 8.47 (s, 1H), 8.33 (s, 1H), 7.86-7.77 (m, 2H), 7.68 (d, 1H), 7.60-7.45 (m, 3H), 7.46-7.39 (m, 1H), 7.39-7.26 (m, 2H), 4.10-4.03 (m, 6H), 3.96-3.92 (m, 2H), 3.05-2.86 (m, 4H), 2.45-2.25 (m, 7H), 1.90-1.81 (m, 2H).

### Example 141: 3-(((((S)-5-Oxopyrrolidin-2-yl)methyl)amino)methyl)-8-(2,2′,5-trichloro-3′-(6-methoxy-5-(((((S)-5-oxopyrrolidin-2-yl)methyl)amino)methyl)pyridin-2-yl)-[1,1′-biphenyl]-3-yl)-4H-pyrido[1,2-a]pyrimidin-4-one

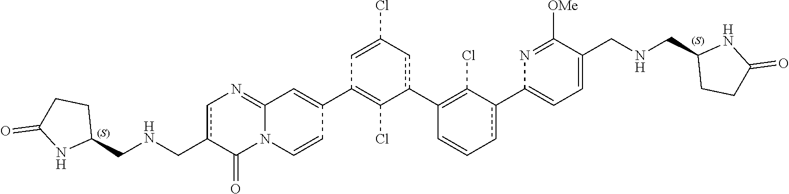

### 6-(3′-Bromo-2,2′,5′-trichloro-[1,1′-biphenyl]-3-yl)-2-methoxynicotinaldehyde

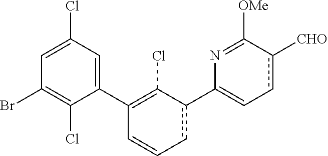

To a solution of 6-(2-chloro-3-(4,4,5,5-tetramethyl-1,3,2-dioxaborolan-2-yl)phenyl)-2-methoxynicotinaldehyde (0.15 g, 0.4 mmol) and 1,3-dibromo-2,5-dichlorobenzene (0.18 g, 0.6 mmol) in dioxane (10 mL) under nitrogen gas was added 1,1′-bis(diphenylphosphino)ferrocene-palladium(ii) dichloride (DCM adduct) (0.03 g, 0.04 mmol) and potassium carbonate (0.14 g, 1.0 mmol). The mixture was stirred at 90° C. for 3 hrs. The mixture was filtered through CELITE® and the filter cake was washed with EtOAc (3×40 mL). The filtrate was washed with saturated aqueous brine solution (2×30 mL), then dried over Na₂SO₄ and concentrated under reduced pressure. The residue was purified by normal phase flash SiO₂ chromatography using a linear gradient (0-40% ethyl acetate hexanes) to afford 6-(3′-bromo-2,2′,5′-trichloro-[1,1′-biphenyl]-3-yl)-2-methoxynicotinaldehyde (0.093 g, 50% yield). MS: m/z found 470 [M+H]⁺.

### 4-Oxo-8-(2,2′,5-trichloro-3′-(5-formyl-6-methoxypyridin-2-yl)-[1,1′-biphenyl]-3-yl)-4H-pyrido[1,2-a]pyrimidine-3-carbaldehyde

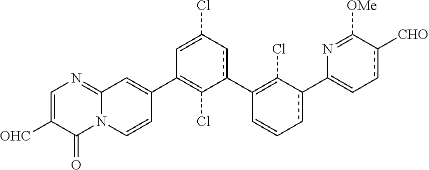

To a solution of 6-(3′-bromo-2,2′,5′-trichloro-[1,1′-biphenyl]-3-yl)-2-methoxynicotinaldehyde (0.09 g, 0.19 mmol) and (3-formyl-4-oxo-4H-pyrido[1,2-a]pyrimidin-8-yl)boronic acid (0.05 g, 0.25 mmol) in dioxane (10 mL) under nitrogen gas was added 1,1′-bis(diphenylphosphino)ferrocene-palladium(ii) dichloride (DCM adduct) (0.01 g, 0.01 mmol) and potassium carbonate (0.07 g, 0.48 mmol). The mixture was stirred at 90° C. for 3 hrs. The mixture was filtered through CELITE® and the filter cake was washed with EtOAc (3×40 mL). The filtrate was washed with saturated aqueous brine solution (2×30 mL), then dried over Na₂SO₄ and concentrated under reduced pressure. The residue was purified by normal phase flash SiO₂ chromatography using a linear gradient (0-3% MeOH/DCM) to afford 4-oxo-8-(2,2′,5-trichloro-3′-(5-formyl-6-methoxypyridin-2-yl)-[1,1′-biphenyl]-3-yl)-4H-pyrido[1,2-a]pyrimidine-3-carbaldehyde (0.027 g, 25% yield). MS: m/z found 564 [M+H]⁺.

### 3-(((((S)-5-Oxopyrrolidin-2-yl)methyl)amino)methyl)-8-(2,2′,5-trichloro-3′-(6-methoxy-5-(((((S)-5-oxopyrrolidin-2-yl)methyl)amino)methyl)pyridin-2-yl)-[1,1′-biphenyl]-3-yl)-4H-pyrido[1,2-a]pyrimidin-4-one

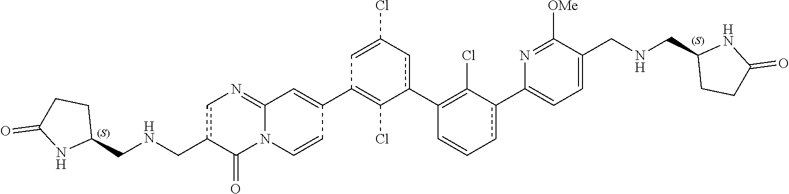

To a mixture of 4-oxo-8-(2,2′,5-trichloro-3′-(5-formyl-6-methoxypyridin-2-yl)-[1,1′-biphenyl]-3-yl)-4H-pyrido[1,2-a]pyrimidine-3-carbaldehyde (0.03 g, 0.05 mmol) and (S)-5-(aminomethyl)pyrrolidin-2-one (0.02 g, 0.17 mmol) in 1:1 THF/MeOH (6 mL) was added acetic acid (0.01 g, 0.14 mmol) and 4 Å molecular sieve (1 g). The mixture was stirred at r.t. for 3 hrs. Sodium cyanoborohydride (0.01 g, 0.17 mmol) was added and the mixture was stirred at r.t. for 1 hr. The reaction was concentrated, and residue purified by Gilson (with 0.05% Formic acid modifier). ACN was evaporated off, and aqueous portion was lyophilized to give 13.3 mg (37%) of the product as bis formic acid salt affording 3-(((((S)-5-oxopyrrolidin-2-yl)methyl)amino)methyl)-8-(2,2′,5-trichloro-3′-(6-methoxy-5-(((((S)-5-oxopyrrolidin-2-yl)methyl)amino)methyl)pyridin-2-yl)-[1,1′-biphenyl]-3-yl)-4H-pyrido[1,2-a]pyrimidin-4-one as a white solid. MS: m/z found 760 [M+H]⁺. ¹H NMR (400 MHz, Methanol-d₄) δ 9.16 (d, J=7.4, 0.8 Hz, 1H), 8.46 (s, 1H), 8.33 (s, 1H), 7.85-7.77 (m, 2H), 7.73-7.65 (m, 2H), 7.60-7.49 (m, 3H), 7.43 (d, J=7.6, 1.8 Hz, 1H), 7.30 (d, J=7.5, 4.0 Hz, 1H), 4.15-3.97 (m, 6H), 3.96-3.91 (m, 2H), 3.04-2.86 (m, 4H), 2.43-2.25 (m, 7H), 1.92-1.79 (m, 2H).

### Example 142: 8-(2,2′-Dichloro-3′-(5-((((1r,3r)-3-hydroxy-3-methylcyclobutyl)amino)methyl)-6-methoxypyridin-2-yl)-[1,1′-biphenyl]-3-yl)-3-(((((S)-5-oxopyrrolidin-2-yl)methyl)amino)methyl)-4H-pyrido[1,2-a]pyrimidin-4-one

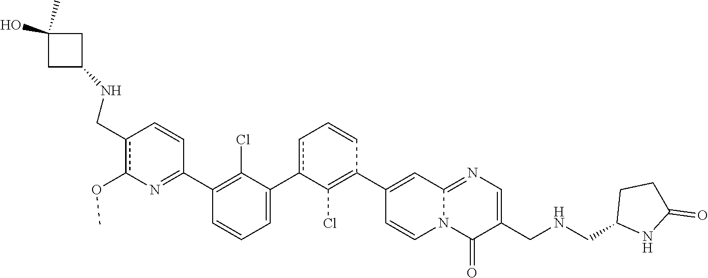

tert-butyl N-[[8-[2-chloro-3-[2-chloro-3-(5-formyl-6-methoxy-2-pyridyl)phenyl]phenyl]-4-oxo-pyrido[1,2-a]pyrimidin-3-yl]methyl]-N-[[(2S)-5-oxopyrrolidin-2-yl]methyl]carbamate (60.00 mg, 0.08 mmol), 3-amino-1-methyl-cyclobutanol;hydrochloride (22.66 mg, 0.16 mmol), DIEA (35.89 μL, 26.56 mg, 0.21 mmol) and acetic acid (9.89 mg, 0.16 mmol) were dissolved in 1 ml of 1:1 MeOH/THF solvent mix, and the reaction was stirred at 25° C. for 3 hours. NaBH₃CN (10.35 mg, 0.16 mmol) was added portion-wise, and the resulting mixture was stirred at 25° C. for 1 hour. The reaction was concentrated and the residue was dissolved in water (3 ml), then extracted with DCM (3×2 ml). The combined organics were further washed with brine, dried over sodium sulfate, then filtered and concentrated to afford tert-butyl N-[[8-[2-chloro-3-[2-chloro-3-[5-[[(3-hydroxy-3-methyl-cyclobutyl)amino]methyl]-6-methoxy-2-pyridyl]phenyl]phenyl]-4-oxo-pyrido[1,2-a]pyrimidin-3-yl]methyl]-N-[[(2S)-5-oxopyrrolidin-2-yl]methyl]carbamate (67.00 mg) as a light yellow foam. Material was used as—is for the next step. MS: m/z found 813, 815 [M+H]⁺.

tert-butyl N-[[8-[2-chloro-3-[2-chloro-3-[5-[[(3-hydroxy-3-methyl-cyclobutyl)amino]methyl]-6-methoxy-2-pyridyl]phenyl]phenyl]-4-oxo-pyrido[1,2-a]pyrimidin-3-yl]methyl]-N-[[(2S)-5-oxopyrrolidin-2-yl]methyl]carbamate (67.00 mg, 0.08 mmol) was dissolved in 0.5 ml DCM and 4M HCl solution in dioxane (82.33 μL, 11.86 mg, 0.33 mmol) was added. The reaction was stirred at rt for 20 minutes and the mixture was subsequently concentrated. Crude sample was purified by reverse phase HPLC to afford 8-[2-chloro-3-[2-chloro-3-[5-[[(3-hydroxy-3-methyl-cyclobutyl)amino]methyl]-6-methoxy-2-pyridyl]phenyl]phenyl]-3-[[[(2S)-5-oxopyrrolidin-2-yl]methylamino]methyl]pyrido[1,2-a]pyrimidin-4-one (30.00 mg, 51.1% yield) as a white fluffy solid (bis formic acid salt). MS: m/z found 713.45, 715.7 [M+H]⁺. ¹H NMR (400 MHz, Methanol-d₄) δ 9.16 (d, J=7.3 Hz, 1H), 8.45 (d, J=8.0 Hz, 3H), 7.86 (d, J=7.5 Hz, 1H), 7.81 (d, J=1.8 Hz, 1H), 7.68-7.49 (m, 6H), 7.45-7.41 (m, 1H), 7.34 (d, J=7.5 Hz, 1H), 4.14 (s, 2H), 4.11-4.05 (m, 5H), 3.93 (dt, J=16.2, 8.0 Hz, 2H), 2.96 (qd, J=12.5, 6.2 Hz, 2H), 2.43-2.29 (m, 5H), 2.21 (t, J=10.8 Hz, 2H), 1.94-1.78 (m, 1H), 1.38 (s, 3H).

### Example 143: 8-(2,2′-Dichloro-3′-(6-methoxy-5-(((((S)-5-oxopyrrolidin-2-yl)methyl)amino)methyl)pyridin-2-yl)-[1,1′-biphenyl]-3-yl)-3-((((1r,3r)-3-hydroxy-3-methylcyclobutyl)amino)methyl)-4H-pyrido[1,2-a]pyrimidin-4-one

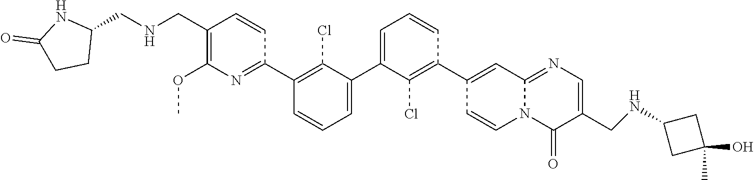

### tert-Butyl ((6-(2,2′-dichloro-3′-(3-((((1r,3r)-3-hydroxy-3-methylcyclobutyl)amino)methyl)-4-oxo-4H-pyrido[1,2-a]-pyrimidin-8-yl)-[1,1′-biphenyl]-3-yl)-2-methoxypyridin-3-yl)methyl)(((S)-5-oxopyrrolidin-2-yl)methyl)carbamate

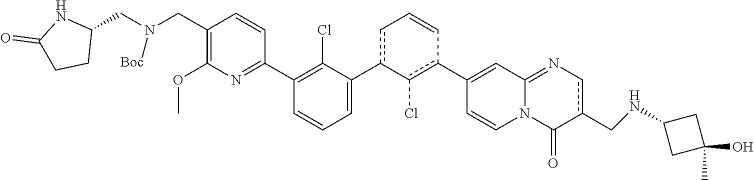

3-Amino-1-methyl-cyclobutanol;hydrochloride (18.89 mg, 0.14 mmol), tert-butyl N-[[6-[2-chloro-3-[2-chloro-3-(3-formyl-4-oxo-pyrido[1,2-a]pyrimidin-8-yl)phenyl]phenyl]-2-methoxy-3-pyridyl]methyl]-N-[[(2S)-5-oxopyrrolidin-2-yl]methyl]carbamate (50 mg, 0.07 mmol) and DIEA (36 μL, 26.56 mg, 0.21 mmol) were dissolved in 1 ml of 1:1 MeOH/THF solvent mix, and the reaction was stirred at 25° C. for 5 hours. Acetic acid, (8 mg, 0.14 mmol) and NaBH₃CN (9 mg, 0.14 mmol) were added, and the resulting mixture was stirred at 25° C. for 1 hour. The reaction was concentrated and the residue was dissolved in water (3 ml), then extracted with DCM (3×2 mL). The combined organics were further washed with brine, dried over sodium sulfate, then filtered and concentrated to afford tert-butyl N-[[6-[2-chloro-3-[2-chloro-3-[3-[[(3-hydroxy-3-methyl-cyclobutyl)amino]methyl]-4-oxo-pyrido[1,2-a]pyrimidin-8-yl]phenyl]phenyl]-2-methoxy-3-pyridyl]methyl]-N-[[(2S)-5-oxopyrrolidin-2-yl]methyl]carbamate (55 mg) as a yellow oil. Material was used as—is for the next step. MS: m/z found 813.4, 815 [M+H]⁺.

### 8-(2,2′-Dichloro-3′-(6-methoxy-5-(((((S)-5-oxopyrrolidin-2-yl)methyl)amino)methyl)pyridin-2-yl)-[1,1′-biphenyl]-3-yl)-3-((((1r,3r)-3-hydroxy-3-methylcyclobutyl)amino)methyl)-4H-pyrido[1,2-a]pyrimidin-4-one

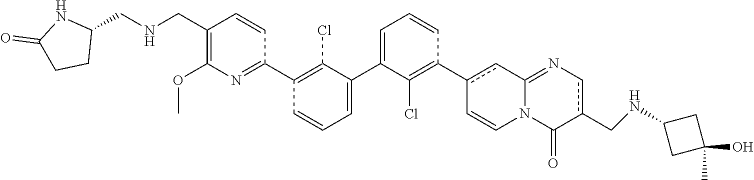

3-Amino-1-methyl-cyclobutanol;hydrochloride (19 mg, 0.14 mmol), tert-butyl N-[[6-[2-chloro-3-[2-chloro-3-(3-formyl-4-oxo-pyrido[1,2-a]pyrimidin-8-yl)phenyl]phenyl]-2-methoxy-3-pyridyl]methyl]-N-[[(2S)-5-oxopyrrolidin-2-yl]methyl]carbamate (50 mg, 0.07 mmol), DIEA (36 μL, 27 mg, 0.21 mmol) and acetic acid (8. mg, 0.14 mmol) were dissolved in 1 ml of 1:1 MeOH/THF solvent mix, and the reaction was stirred at 25° C. for 3 hours. NaBH₃CN (9 mg, 0.14 mmol) was added portion-wise, and the resulting mixture was stirred at 25° C. for 1 hour. The reaction was concentrated and the sample was dissolved in water (3 ml), then extracted with DCM (3×2 mL). The combined organics were further washed with brine, dried over sodium sulfate, then filtered and concentrated to afford tert-butyl N-[[6-[2-chloro-3-[2-chloro-3-[3-[[(3-hydroxy-3-methyl-cyclobutyl)amino]methyl]-4-oxo-pyrido[1,2-a]pyrimidin-8-yl]phenyl]phenyl]-2-methoxy-3-pyridyl]methyl]-N-[[(2S)-5-oxopyrrolidin-2-yl]methyl]carbamate (55 mg) as a yellow oil. Material was used as—is for the next step. MS: m/z found 813.4, 815 [M+H]⁺.

tert-butyl N-[[6-[2-chloro-3-[2-chloro-3-[3-[[(3-hydroxy-3-methyl-cyclobutyl)amino]methyl]-4-oxo-pyrido[1,2-a]pyrimidin-8-yl]phenyl]phenyl]-2-methoxy-3-pyridyl]methyl]-N-[[(2S)-5-oxopyrrolidin-2-yl]methyl]carbamate (55 mg, 0.07 mmol) was dissolved in 0.5 ml DCM and 4M HCl solution in dioxane (68 μL, 9.73 mg, 0.27 mmol) was added. The reaction was stirred at rt for 20 minutes and the mixture was subsequently concentrated. Crude residue was purified by reverse phase HPLC (5-50% ACN gradient in water, with 0.05% formic acid). The combined pure product fractions were lypholized to afford 8-[2-chloro-3-[2-chloro-3-[6-methoxy-5-[[[(2S)-5-oxopyrrolidin-2-yl]methylamino]methyl]-2-pyridyl]phenyl]phenyl]-3-[[(3-hydroxy-3-methyl-cyclobutyl)amino]methyl]pyrido[1,2-a]pyrimidin-4-one (19.7 mg, 17.7% yield) as a white fluffy solid (bis formic acid salts). MS: m/z found 713.4, 715.4 [M+H]⁺. ¹H NMR (400 MHz, Methanol-d₄) δ 9.18 (d, J=7.4 Hz, 1H), 8.50 (s, 1H), 8.44 (s, 1H), 7.85 (d, J=1.8 Hz, 1H), 7.81 (d, J=7.5 Hz, 1H), 7.71-7.58 (m, 4H), 7.57-7.48 (m, 2H), 7.41 (dd, J=7.6, 1.7 Hz, 1H), 7.29 (d, J=7.4 Hz, 1H), 4.16 (s, 2H), 4.05 (s, 5H), 3.95 (q, J=8.2 Hz, 2H), 2.99-2.87 (m, 2H), 2.47-2.30 (m, 5H), 2.29-2.20 (m, 2H), 1.92-1.79 (m, 1H), 1.39 (s, 3H).

### Example 144: 8-(2,2′-Dichloro-3′-(6-methoxy-5-(((((S)-5-oxopyrrolidin-2-yl)methyl)amino)methyl)pyridin-2-yl)-[1,1′-biphenyl]-3-yl)-3-(((S)-3-hydroxypyrrolidin-1-yl)methyl)-4H-pyrido[1,2-a]pyrimidin-4-one

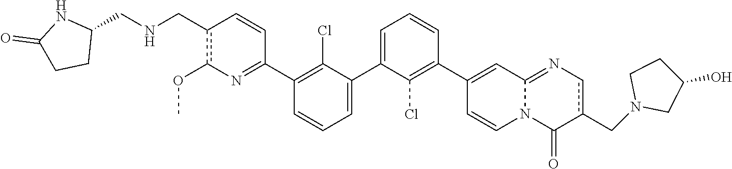

tert-butyl N-[[6-[2-chloro-3-[2-chloro-3-(3-formyl-4-oxo-pyrido[1,2-a]pyrimidin-8-yl)phenyl]phenyl]-2-methoxy-3-pyridyl]methyl]-N-[[(2S)-5-oxopyrrolidin-2-yl]methyl]carbamate (38 mg, 0.05 mmol), (3S)-pyrrolidin-3-ol (4.5 mg, 0.05 mmol) and acetic acid (6 mg, 0.10 mmol) were dissolved in 1 ml of 1:1 MeOH/THF solvent mix, and the reaction was stirred at 80° C. for 5 hours. NaBH₃CN (7 mg, 0.10 mmol) was added portion-wise, and the resulting mixture was stirred at 25° C. for 1 hour. The reaction was concentrated and the sample was dissolved in water (3 mL), then extracted with DCM (3×2 mL). The combined organics were further washed with brine, dried over sodium sulfate, then filtered and concentrated to afford tert-butyl N-[[6-[2-chloro-3-[2-chloro-3-[3-[[(3S)-3-hydroxypyrrolidin-1-yl]methyl]-4-oxo-pyrido[1,2-a]pyrimidin-8-yl]phenyl]phenyl]-2-methoxy-3-pyridyl]methyl]-N-[[(2S)-5-oxopyrrolidin-2-yl]methyl]carbamate (41 mg, 59.0% yield) as an orange oil. Material was used as—is for the next step. MS: m/z found 799, 801 [M+H]⁺.

tert-butyl N-[[6-[2-chloro-3-[2-chloro-3-[3-[[(3S)-3-hydroxypyrrolidin-1-yl]methyl]-4-oxo-pyrido[1,2-a]pyrimidin-8-yl]phenyl]phenyl]-2-methoxy-3-pyridyl]methyl]-N-[[(2S)-5-oxopyrrolidin-2-yl]methyl]carbamate (41 mg, 0.05 mmol) was dissolved in 0.5 ml DCM and 4M HCl solution in dioxane (51 μL, 0.21 mmol) was added. The reaction was stirred at rt for 20 minutes and the mixture was subsequently concentrated. Crude sample was purified by reverse phase HPLC to afford 8-[2-chloro-3-[2-chloro-3-[6-methoxy-5-[[[(2S)-5-oxopyrrolidin-2-yl]methylamino]methyl]-2-pyridyl]phenyl]phenyl]-3-[[(3S)-3-hydroxypyrrolidin-1-yl]methyl]pyrido[1,2-a]pyrimidin-4-one (4.4 mg, 12.3% yield) as a yellow foam. MS: m/z found 699.4, 701 [M+H]⁺. ¹H NMR (400 MHz, Chloroform-d) δ 9.10 (d, J=7.4 Hz, 1H), 8.44 (s, 1H), 7.76 (d, J=1.9 Hz, 1H), 7.69 (dd, J=7.6, 1.8 Hz, 1H), 7.59 (d, J=7.4 Hz, 1H), 7.52-7.42 (m, 4H), 7.35 (ddd, J=14.3, 7.4, 1.8 Hz, 2H), 7.28 (s, 1H), 6.02 (s, 1H), 4.37 (d, J=6.4 Hz, 1H), 4.03 (s, 3H), 3.85-3.75 (m, 5H), 3.09-2.99 (m, 1H), 2.89-2.77 (m, 2H), 2.71 (dd, J=10.2, 5.2 Hz, 1H), 2.53 (ddd, J=29.3, 14.1, 8.5 Hz, 2H), 2.37 (dd, J=9.0, 7.1 Hz, 2H), 2.25 (dt, J=13.2, 7.3 Hz, 2H), 1.83-1.75 (m, 2H), 0.85 (s, 1H).

### Example 145: 2′-Chloro-3-(6-methoxy-5-(((((S)-5-oxopyrrolidin-2-yl)methyl)amino)methyl)pyridin-2-yl)-3′-(4-oxo-3-(((((S)-5-oxopyrrolidin-2-yl)methyl)amino)methyl)-4H-pyrido[1,2-a]pyrimidin-8-yl)-[1,1′-biphenyl]-2-carbonitrile

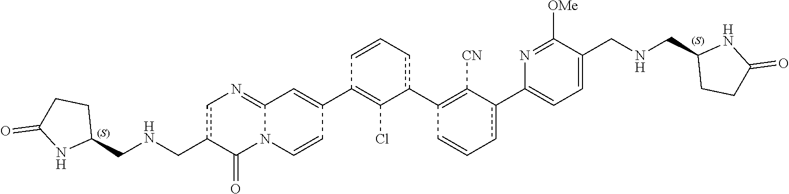

### 3-Bromo-2′-chloro-3′-(3-formyl-4-oxo-4H-pyrido[1,2-a]pyrimidin-8-yl)-[1,1′-biphenyl]-2-carbonitrile

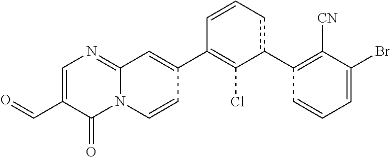

To a solution of 8-(2-chloro-3-(4,4,5,5-tetramethyl-1,3,2-dioxaborolan-2-yl)phenyl)-4-oxo-4H-pyrido[1,2-a]pyrimidine-3-carbaldehyde (0.13 g, 0.32 mmol) and 2,6-dibromobenzonitrile (0.12 g, 0.47 mmol) in dioxane (10 mL) under nitrogen gas was added 1,1′-bis(diphenylphosphino)ferrocene-palladium(ii) dichloride (DCM adduct) (0.02 g, 0.02 mmol) and potassium carbonate (0.11 g, 0.79 mmol). The mixture was stirred at 90° C. for 3 hrs. The mixture was filtered through CELITE® and the filter cake was washed with EtOAc (3×40 mL). The filtrate was washed with saturated aqueous brine solution (2×30 mL), then dried over Na₂SO₄ and concentrated under reduced pressure. The residue was purified by normal phase flash SiO₂ chromatography using a linear gradient (0-40% ethyl acetate hexanes) to afford 3-bromo-2′-chloro-3′-(3-formyl-4-oxo-4H-pyrido[1,2-a]pyrimidin-8-yl)-[1,1′-biphenyl]-2-carbonitrile (0.034 g, 23% yield). MS: m/z found 464 [M+H]⁺.

### 2′-Chloro-3′-(3-formyl-4-oxo-4H-pyrido[1,2-a]pyrimidin-8-yl)-3-(5-formyl-6-methoxypyridin-2-yl)-[1,1′-biphenyl]-2-carbonitrile

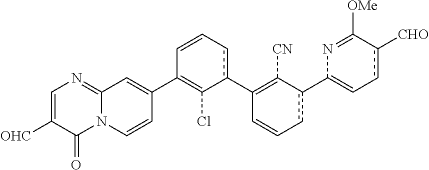

To a solution of 3-bromo-2′-chloro-3′-(3-formyl-4-oxo-4H-pyrido[1,2-a]pyrimidin-8-yl)-[1,1′-biphenyl]-2-carbonitrile (0.03 g, 0.07 mmol) and (5-formyl-6-methoxypyridin-2-yl)boronic acid (0.03 g, 0.15 mmol) in dioxane (10 mL) under nitrogen gas was added 1,1′-bis(diphenylphosphino)ferrocene-palladium(ii) dichloride (DCM adduct) (0.01 g, 0.01 mmol) and potassium carbonate (0.03 g, 0.18 mmol). The mixture was stirred at 90° C. for 3 hrs. The mixture was filtered through CELITE® and the filter cake was washed with EtOAc (3×40 mL). The filtrate was washed with saturated aqueous brine solution (2×30 mL), then dried over Na₂SO₄ and concentrated under reduced pressure. The residue was purified by normal phase flash SiO₂ chromatography using a linear gradient (0-3% MeOH/DCM) to afford 2′-chloro-3′-(3-formyl-4-oxo-4H-pyrido[1,2-a]pyrimidin-8-yl)-3-(5-formyl-6-methoxypyridin-2-yl)-[1,1′-biphenyl]-2-carbonitrile (0.02 g, 50% yield). MS: m/z found 521 [M+H]⁺. 2′-Chloro-3-(6-methoxy-5-(((((S)-5-oxopyrrolidin-2-yl)methyl)amino)methyl)pyridin-2-yl)-3′-(4-oxo-3-(((((S)-5-oxopyrrolidin-2-yl)methyl)amino)methyl)-4H-pyrido[1,2-a]pyrimidin-8-yl)-[1,1′-biphenyl]-2-carbonitrile

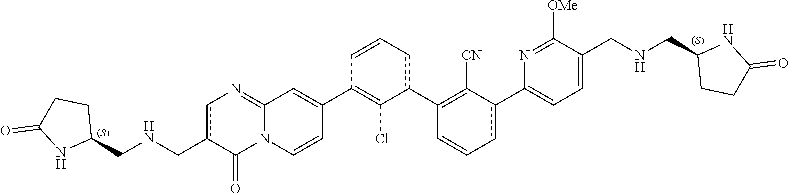

To a mixture of 2′-chloro-3′-(3-formyl-4-oxo-4H-pyrido[1,2-a]pyrimidin-8-yl)-3-(5-formyl-6-methoxypyridin-2-yl)-[1,1′-biphenyl]-2-carbonitrile (0.02 g, 0.04 mmol) and (S)-5-20 (aminomethyl)pyrrolidin-2-one (0.02 g, 0.13 mmol) in 1:1 THIF/MeOH (6 mL) was added acetic acid (0.01 g, 0.12 mmol) and 4 Å molecular sieve (1 g). The mixture was stirred at r.t. for 3 hrs. Sodium cyanoborohydride (0.01 g, 0.13 mmol) was added and the mixture was stirred at r.t. for 1 hr. The reaction was concentrated, and residue purified by Gilson (with 0.05% Formic acid modifier). ACN was evaporated off, and aqueous portion was lyophilized to give 19 mg (68%) of the product as bis formic acid salt affording 2′-chloro-3-(6-methoxy-5-(((((S)-5-oxopyrrolidin-2-yl)methyl)amino)methyl)pyridin-2-yl)-3′-(4-oxo-3-(((((S)-5-oxopyrrolidin-2-yl)methyl)amino)methyl)-4H-pyrido[1,2-a]pyrimidin-8-yl)-[1,1′-biphenyl]-2-carbonitrile as a white solid. MS: m/z found 717 [M+H]⁺. ¹H NMR (400 MHz, Methanol-d₄) δ 9.15 (dd, J=7.4, 0.8 Hz, 1H), 8.43 (s, 1H), 7.95 (d, J=8.0, 1.2 Hz, 1H), 7.89-7.78 (m, 3H), 7.73-7.57 (m, 5H), 7.47 (d, J=7.5 Hz, 1H), 4.10 (s, 3H), 4.02-3.82 (m, 6H), 2.98-2.66 (m, 3H), 2.41-2.23 (m, 7H), 1.89-1.75 (m, 2H).

### Example 146: 8-(2,2′-Dichloro-5′-methoxy-3′-(6-methoxy-5-(((((S)-5-oxopyrrolidin-2-yl)methyl)amino)methyl)pyridin-2-yl)-[1,1′-biphenyl]-3-yl)-3-(((((S)-5-oxopyrrolidin-2-yl)methyl)amino)methyl)-4H-pyrido[1,2-a]pyrimidin-4-one

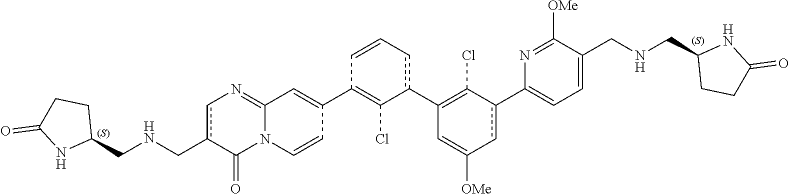

### 8-(3′-Bromo-2,2′-dichloro-5′-methoxy-[1,1′-biphenyl]-3-yl)-4-oxo-4H-pyrido[1,2-a]pyrimidine-3-carbaldehyde

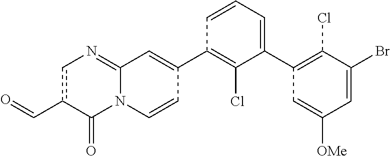

To a solution of 8-(2-chloro-3-(4,4,5,5-tetramethyl-1,3,2-dioxaborolan-2-yl)phenyl)-4-oxo-4H-pyrido[1,2-a]pyrimidine-3-carbaldehyde (0.13 g, 0.32 mmol) and 1,3-dibromo-2-chloro-5-methoxybenzene (0.14 g, 0.47 mmol) in dioxane (10 mL) under nitrogen gas was added 1,1′-bis(diphenylphosphino)ferrocene-palladium(ii) dichloride (DCM adduct) (0.02 g, 0.02 mmol) and potassium carbonate (0.11 g, 0.79 mmol). The mixture was stirred at 90° C. for 3 hrs. The mixture was filtered through CELITE® and the filter cake was washed with EtOAc (3×40 mL). The filtrate was washed with saturated aqueous brine solution (2×30 mL), then dried over Na₂SO₄ and concentrated under reduced pressure. The residue was purified by normal phase flash SiO₂ chromatography using a linear gradient (0-40% ethyl acetate hexanes) to afford 8-(3′-bromo-2,2′-dichloro-5′-methoxy-[1,1′-biphenyl]-3-yl)-4-oxo-4H-pyrido[1,2-a]pyrimidine-3-carbaldehyde (0.30 g, 19% yield). MS: m/z found 494 [M+H]⁺.

### 8-(2,2′-Dichloro-3′-(5-formyl-6-methoxypyridin-2-yl)-5′-methoxy-[1,1′-biphenyl]-3-yl)-4-oxo-4H-pyrido[1,2-a]pyrimidine-3-carbaldehyde

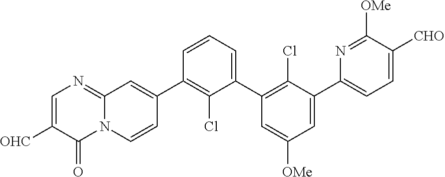 [SMILES: COc1cc(-c2ccc(C=O)c(OC)n2)c(Cl)c(-c2cccc(-c3ccn4c(=O)c(C=O)cnc4c3)c2Cl)c1]

To a solution of 8-(3′-bromo-2,2′-dichloro-5′-methoxy-[1,1′-biphenyl]-3-yl)-4-oxo-4H-pyrido[1,2-a]pyrimidine-3-carbaldehyde (0.03 g, 0.07 mmol) and (5-formyl-6-methoxypyridin-2-yl)boronic acid (0.02 g, 0.12 mmol) in dioxane (10 mL) under nitrogen gas was added 1,1′-bis(diphenylphosphino)ferrocene-palladium(ii) dichloride (DCM adduct) (0.01 g, 0.01 mmol) and potassium carbonate (0.02 g, 0.15 mmol). The mixture was stirred at 90° C. for 3 hrs. The mixture was filtered through CELITE® and the filter cake was washed with EtOAc (3×40 mL). The filtrate was washed with saturated aqueous brine solution (2×30 mL), then dried over Na₂SO₄ and concentrated under reduced pressure. The residue was purified by normal phase flash SiO₂ chromatography using a linear gradient (0-3% MeOH/DCM) to afford 8-(2,2′-dichloro-3′-(5-formyl-6-methoxypyridin-2-yl)-5′-methoxy-[1,1′-biphenyl]-3-yl)-4-oxo-4H-pyrido[1,2-a]pyrimidine-3-carbaldehyde (0.011 g, 33% yield). MS: m/z found 560 [M+H]⁺.

### 8-(2,2′-Dichloro-5′-methoxy-3′-(6-methoxy-5-(((((S)-5-oxopyrrolidin-2-yl)methyl)amino)methyl)pyridin-2-yl)-[1,1′-biphenyl]-3-yl)-3-(((((S)-5-oxopyrrolidin-2-yl)methyl)amino)methyl)-4H-pyrido[1,2-a]pyrimidin-4-one

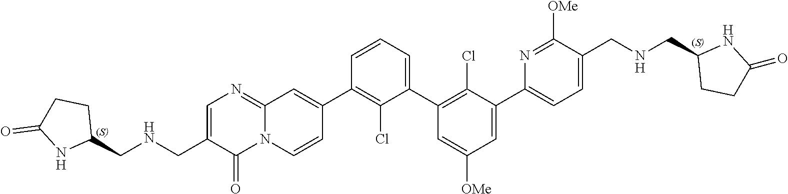

To a mixture of 8-(2,2′-dichloro-3′-(5-formyl-6-methoxypyridin-2-yl)-5′-methoxy-[1,1′-biphenyl]-3-yl)-4-oxo-4H-pyrido[1,2-a]pyrimidine-3-carbaldehyde (0.01 g, 0.02 mmol) and (S)-5-(aminomethyl)pyrrolidin-2-one (0.01 g, 0.06 mmol) in 1:1 THF/MeOH (6 mL) was added acetic acid (0.004 g, 0.05 mmol) and 4 Å molecular sieve (1 g). The mixture was stirred at r.t. for 3 hrs. Sodium cyanoborohydride (0.004 g, 0.06 mmol) was added and the mixture was stirred at r.t. for 1 hr. The reaction was concentrated, and residue purified by Gilson (with 0.05% Formic acid modifier). ACN was evaporated off, and aqueous portion was lyophilized to give 8 mg (57%) of the product as bis formic acid salt affording 8-(2,2′-dichloro-5′-methoxy-3′-(6-methoxy-5-(((((S)-5-oxopyrrolidin-2-yl)methyl)amino)methyl)pyridin-2-yl)-[1,1′-biphenyl]-3-yl)-3-(((((S)-5-oxopyrrolidin-2-yl)methyl)amino)methyl)-4H-pyrido[1,2-a]pyrimidin-4-one as a white solid. MS: m/z found 756 [M+H]⁺. ¹H NMR (400 MHz, Methanol-d₄) δ 9.13 (dd, J=7.4, 0.8 Hz, 1H), 8.52 (s, 1H), 8.40 (s, 1H), 7.78-7.68 (m, 2H), 7.64-7.46 (m, 3H), 7.27-7.17 (m, 2H), 6.96 (d, J=3.1 Hz, 1H), 4.01 (s, 3H), 3.93-3.76 (m, 9H), 2.80-2.62 (m, 4H), 2.37-2.27 (m, 5H), 2.25 (dd, J=14.8, 7.3 Hz, 2H), 1.85-1.76 (m, 1H).

### Example 147: 8-(2,2′-Dichloro-3′-(4-fluoro-6-methoxy-5-(((((S)-5-oxopyrrolidin-2-yl)methyl)amino)methyl)pyridin-2-yl)-[1,1′-biphenyl]-3-yl)-3-(((((S)-5-oxopyrrolidin-2-yl)methyl)amino)methyl)-4H-pyrido[1,2-a]pyrimidin-4-one

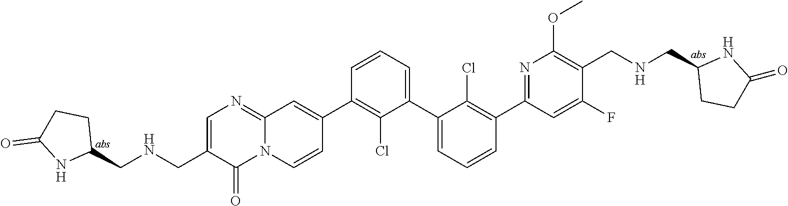

### 8-2-Chloro-3-[2-chloro-3-(4-fluoro-5-formyl-6-methoxy-2-pyridyl)phenylphenyl-4-oxo-pyrido[1,2-a]pyrimidine-3-carbaldehyde

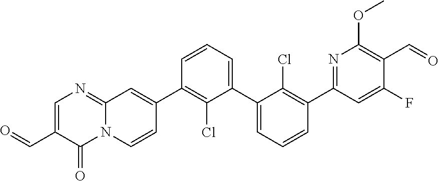 [SMILES: COc1nc(-c2cccc(-c3cccc(-c4ccn5c(=O)c(C=O)cnc5c4)c3Cl)c2Cl)cc(F)c1C=O]

To a solution of 8-[2-chloro-3-[2-chloro-3-(4,4,5,5-tetramethyl-1,3,2-dioxaborolan-2-yl)phenyl]phenyl]-4-oxo-pyrido[1,2-a]pyrimidine-3-carbaldehyde (100 mg, 0.19 mmol), 6-chloro-4-fluoro-2-methoxy-pyridine-3-carbaldehyde (47 mg, 0.25 mmol) and potassium carbonate (80 mg, 0.58 mmol) in degassed dioxane/water (6:1) was added Pd(PPh₃)₄ (22 mg, 0.02 mmol) and the mixture was stirred at 90° C. for 4 hours under N₂ (g). Water was added and the mixture was extracted into EtOAc three times. The combined organic layers were dried over anhydrous sodium sulfate, concentrated, and purified on silica gel column using EtOAc in hexanes (0 to 60% gradient) to provide 8-[2-chloro-3-[2-chloro-3-(4-fluoro-5-formyl-6-methoxy-2-pyridyl)phenyl]phenyl]-4-oxo-pyrido[1,2-a]pyrimidine-3-carbaldehyde (75 mg, 71.3%). MS: m/z found 548.1 [M+H]⁺.

### 8-[2-Chloro-3-[2-chloro-3-[4-fluoro-6-methoxy-5-[[[(2S)-5-oxopyrrolidin-2-yl]methylamino]methyl]-2-pyridyl]phenyl]phenyl]-3-[[[(2S)-5-oxopyrrolidin-2-yl]methylamino]methyl]pyrido[1,2-a]pyrimidin-4-one

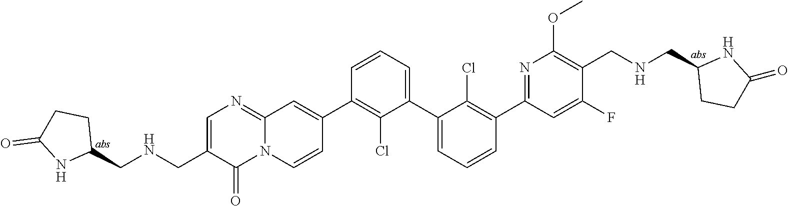

A mixture of 8-[2-chloro-3-[2-chloro-3-(4-fluoro-5-formyl-6-methoxy-2-pyridyl)phenyl]phenyl]-4-oxo-pyrido[1,2-a]pyrimidine-3-carbaldehyde (50 mg, 0.09 mmol), (5S)-5-(aminomethyl)pyrrolidin-2-one (42 mg, 0.36 mmol) and acetic acid (10 uL, 11 mg, 0.18 mmol) in MeOH/THF (1:1) was stirred for 1.5 hour at rt. NaBH₃CN (23 mg, 0.36 mmol) was added, the mixture was stirred for additional 5 hrs, quenched with water and directly purified by HPLC to give 8-[2-chloro-3-[2-chloro-3-[4-fluoro-6-methoxy-5-[[[(2S)-5-oxopyrrolidin-2-yl]methylamino]methyl]-2-pyridyl]phenyl]phenyl]-3-[[[(2S)-5-oxopyrrolidin-2-yl]methylamino]methyl]pyrido[1,2-a]pyrimidin-4-one (58 mg, 85.4%). MS: m/z found 744.2 [M+H]⁺. ¹H NMR (400 MHz, Methanol-d₄): δ 9.15 (dt, J=7.4, 0.7 Hz, 1H), 8.43 (s, 1H), 7.79 (dd, J=1.9, 0.7 Hz, 1H), 7.69 (ddd, J=7.7, 1.8, 0.6 Hz, 1H), 7.66-7.58 (m, 2H), 7.57-7.49 (m, 3H), 7.43 (ddd, J=7.6, 1.7, 0.5 Hz, 1H), 7.13 (d, J=9.2 Hz, 1H), 4.05 (s, 3H), 3.94 (d, J=4.3 Hz, 2H), 3.91 (s, 2H), 3.90-3.86 (m, 1H), 3.86-3.79 (m, 1H), 2.87-2.64 (m, 4H), 2.42-2.21 (m, 6H), 1.89-1.74 (m, 2H).

### Example 148: (2S,4S)-1-((8-(2,2′-Dichloro-3′-(6-methoxy-5-(((((S)-5-oxopyrrolidin-2-yl)methyl)amino)methyl)pyridin-2-yl)-[1,1′-biphenyl]-3-yl)-4-oxo-4H-pyrido[1,2-a]pyrimidin-3-yl)methyl)-4-hydroxypyrrolidine-2-carboxylic acid

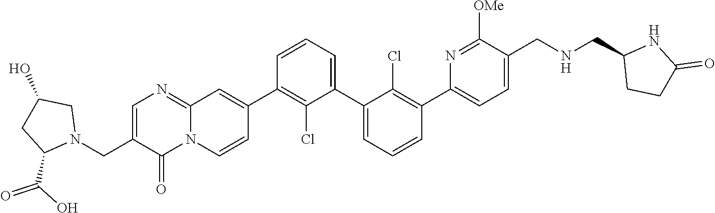 [SMILES: COc1nc(-c2cccc(-c3cccc(-c4ccn5c(=O)c(CN6C[C@@H](O)C[C@H]6C(=O)O)cnc5c4)c3Cl)c2Cl)ccc1CNC[C@@H]1CCC(=O)N1]

### (2S,4S)-1-((8-(3′-(5-(((tert-Butoxycarbonyl)(((S)-5-oxopyrrolidin-2-yl)methyl)amino)methyl)-6-methoxypyridin-2-yl)-2,2′-dichloro-[1,1′-biphenyl]-3-yl)-4-oxo-4H-pyrido[1,2-a]pyrimidin-3-yl)methyl)-4-hydroxypyrrolidine-2-carboxylic acid

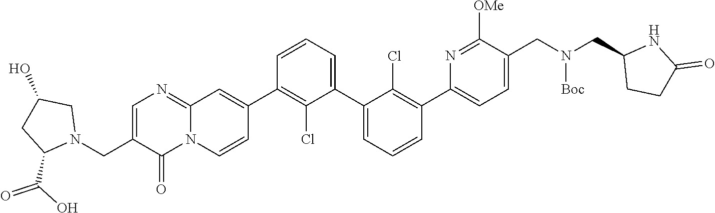 [SMILES: COc1nc(-c2cccc(-c3cccc(-c4ccn5c(=O)c(CN6C[C@@H](O)C[C@H]6C(=O)O)cnc5c4)c3Cl)c2Cl)ccc1CN(C[C@@H]1CCC(=O)N1)C(=O)OC(C)(C)C]

A mixture of (S)-tert-butyl ((6-(2,2′-dichloro-3′-(3-formyl-4-oxo-4H-pyrido[1,2-a]pyrimidin-8-yl)-[1,1′-biphenyl]-3-yl)-2-methoxypyridin-3-yl)methyl)((5-oxopyrrolidin-2-yl)methyl)carbamate (0.28 g, 0.38 mmol), (2S,4S)-4-hydroxypyrrolidine-2-carboxylic acid (0.1 g, 0.77 mmol), glacial acetic acid (1 mL, 0.17 mmol) and sodium cyanoborohydride (0.07 g, 1.15 mmol) in DMF (2 mL) was stirred at 60° C. for 2 hrs under nitrogen atmosphere. The mixture was used for next step directly without work-up. (2S,4S)-1-((8-(3′-(5-(((tert-butoxycarbonyl)(((S)-5-oxopyrrolidin-2-yl)methyl)amino)methyl)-6-methoxypyridin-2-yl)-2,2′-dichloro-[1,1′-biphenyl]-3-yl)-4-oxo-4H-pyrido[1,2-a]pyrimidin-3-yl)methyl)-4-hydroxypyrrolidine-2-carboxylic acid was obtained as a yellow liquid (2 mL of DMF solution, m/z: 843 [M+H]⁺).

### (2S,4S)-1-((8-(2,2′-Dichloro-3′-(6-methoxy-5-(((((S)-5-oxopyrrolidin-2-yl)methyl)amino)methyl)pyridin-2-yl)-[1,1′-biphenyl]-3-yl)-4-oxo-4H-pyrido[1,2-a]pyrimidin-3-yl)methyl)-4-hydroxypyrrolidine-2-carboxylic acid

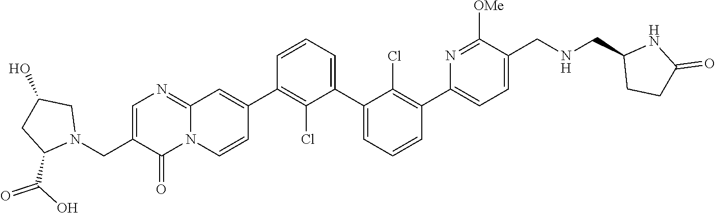 [SMILES: COc1nc(-c2cccc(-c3cccc(-c4ccn5c(=O)c(CN6C[C@@H](O)C[C@H]6C(=O)O)cnc5c4)c3Cl)c2Cl)ccc1CNC[C@@H]1CCC(=O)N1]

To a mixture of (2S,4S)-1-((8-(3′-(5-(((tert-butoxycarbonyl)(((S)-5-oxopyrrolidin-2-yl)methyl)amino)methyl)-6-methoxypyridin-2-yl)-2,2′-dichloro-[1,1′-biphenyl]-3-yl)-4-oxo-4H-pyrido[1,2-a]pyrimidin-3-yl)methyl)-4-hydroxypyrrolidine-2-carboxylic acid in DMF (2 mL) was added trifluoroacetic acid (4 mL, 54 mmol). The mixture was stirred at r.t. for 0.5 h under nitrogen atmosphere. The mixture was concentrated. The residue was purified by reverse phase HPLC to afford (2S,4S)-1-((8-(2,2′-dichloro-3′-(6-methoxy-5-(((((S)-5-oxopyrrolidin-2-yl)methyl)amino)methyl)pyridin-2-yl)-[1,1′-biphenyl]-3-yl)-4-oxo-4H-pyrido[1,2-a]pyrimidin-3-yl)methyl)-4-hydroxypyrrolidine-2-carboxylic acid as a white solid (78.5 mg, 27% yield, m/z: 743 [M+H]⁺). ¹H NMR (400 MHz, Methanol-d₄): δ 9.16 (d, J=6.8 Hz, 1H), 8.54 (s, 1H), 7.86-7.83 (m, 2H), 7.66 (d, J=7.2 Hz, 1H), 7.61-7.58 (m, 3H), 7.54-7.50 (m, 2H), 7.41 (d, J=7.2 Hz, 1H), 7.32 (d, J=6.8 Hz, 1H), 4.48 (m, 3H), 4.18-4.15 (m, 3H), 4.06 (s, 3H), 4.01 (m, 1H), 3.65 (d, J=11.6 Hz, 1H), 3.38 (d, J=11.2 Hz, 1H), 3.10 (m, 2H), 2.69-2.62 (m, 1H), 2.41-2.26 (m, 4H), 1.90-1.88 (m, 1H).

### Example 149: (2R,4S)-1-((8-(2,2′-Dichloro-3′-(6-methoxy-5-(((((S)-5-oxopyrrolidin-2-yl)methyl)amino)methyl)pyridin-2-yl)-[1,1′-biphenyl]-3-yl)-4-oxo-4H-pyrido[1,2-a]pyrimidin-3-yl)methyl)-4-hydroxypyrrolidine-2-carboxylic acid

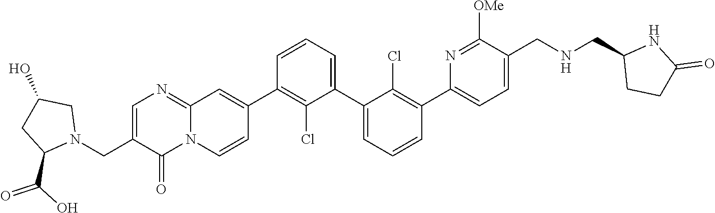 [SMILES: COc1nc(-c2cccc(-c3cccc(-c4ccn5c(=O)c(CN6C[C@@H](O)C[C@@H]6C(=O)O)cnc5c4)c3Cl)c2Cl)ccc1CNC[C@@H]1CCC(=O)N1]

### (2R,4S)-1-((8-(3′-(5-(((tert-Butoxycarbonyl)(((S)-5-oxopyrrolidin-2-yl)methyl)amino)methyl)-6-methoxypyridin-2-yl)-2,2′-dichloro-[1,1′-biphenyl]-3-yl)-4-oxo-4H-pyrido[1,2-a]pyrimidin-3-yl)methyl)-4-hydroxypyrrolidine-2-carboxylic acid

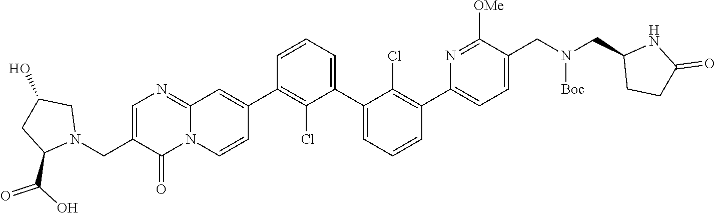 [SMILES: COc1nc(-c2cccc(-c3cccc(-c4ccn5c(=O)c(CN6C[C@@H](O)C[C@@H]6C(=O)O)cnc5c4)c3Cl)c2Cl)ccc1CN(C[C@@H]1CCC(=O)N1)C(=O)OC(C)(C)C]

To a solution of (S)-tert-butyl ((6-(2,2′-dichloro-3′-(3-formyl-4-oxo-4H-pyrido[1,2-a]pyrimidin-8-yl)-[1,1′-biphenyl]-3-yl)-2-methoxypyridin-3-yl)methyl)((5-oxopyrrolidin-2-yl)methyl)carbamate (300 mg, 0.41 mmol) and (2R,4S)-4-hydroxypyrrolidine-2-carboxylic acid (162 mg, 1.24 mmol) in DMF (3 mL) was added sodium cyanoborohydride (77.62 mg, 1.24 mmol, 3 eq) and glacial AcOH (0.02 mL, 0.41 mmol). The mixture was stirred at 60° C. for 2 hr. The reaction mixture was filtered and concentrated. Compound (2R,4S)-1-((8-(3′-(5-(((tert-butoxycarbonyl)(((S)-5-oxopyrrolidin-2-yl)methyl)amino)methyl)-6-methoxypyridin-2-yl)-2,2′-dichloro-[1,1′-biphenyl]-3-yl)-4-oxo-4H-pyrido[1,2-a]pyrimidin-3-yl)methyl)-4-hydroxypyrrolidine-2-carboxylic acid (350 mg, crude, m/z: 843 [M+H]⁺) was obtained as a black oil.

### (2R,4S)-1-((8-(2,2′-Dichloro-3′-(6-methoxy-5-(((((S)-5-oxopyrrolidin-2-yl)methyl)amino)methyl)pyridin-2-yl)-[1,1′-biphenyl]-3-yl)-4-oxo-4H-pyrido[1,2-a]pyrimidin-3-yl)methyl)-4-hydroxypyrrolidine-2-carboxylic acid

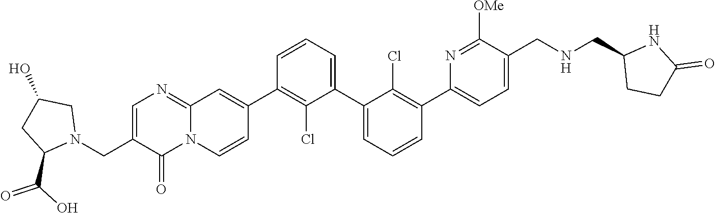 [SMILES: COc1nc(-c2cccc(-c3cccc(-c4ccn5c(=O)c(CN6C[C@@H](O)C[C@@H]6C(=O)O)cnc5c4)c3Cl)c2Cl)ccc1CNC[C@@H]1CCC(=O)N1]

To a solution of (2R,4S)-1-((8-(3′-(5-(((tert-butoxycarbonyl)(((S)-5-oxopyrrolidin-2-yl)methyl)amino)methyl)-6-methoxypyridin-2-yl)-2,2′-dichloro-[1,1′-biphenyl]-3-yl)-4-oxo-4H-pyrido[1,2-a]pyrimidin-3-yl)methyl)-4-hydroxypyrrolidine-2-carboxylic acid (300 mg, 0.36 mmol) in DMF (0.5 mL) was added trifluoroacetic acid (3.00 mL, 40.5 mmol). The mixture was stirred at r.t. for 0.5 hr. The reaction mixture was concentrated. The residue was purified by reverse phase HPLC to afford (2R,4S)-1-((8-(2,2′-dichloro-3′-(6-methoxy-5-(((((S)-5-oxopyrrolidin-2-yl)methyl)amino)methyl)pyridin-2-yl)-[1,1′-biphenyl]-3-yl)-4-oxo-4H-pyrido[1,2-a]pyrimidin-3-yl)methyl)-4-hydroxypyrrolidine-2-carboxylic acid (70.5 mg, 26% yield, m/z: 727 [M+H]⁺) as a white solid. ¹H NMR (400 MHz, Methanol-d₄) δ 9.20 (d, J=7.2 Hz, 1H), 8.58 (s, 1H), 7.86 (d, J=1.6 Hz, 1H), 7.83 (d, J=7.6 Hz, 1H), 7.69-7.62 (m, 4H), 7.56-7.52 (m, 2H), 7.43 (dd, J=7.2 Hz, J=1.6 Hz, 1H), 7.32 (d, J=7.6 Hz, 1H), 4.61-4.56 (m, 3H), 4.40-4.36 (m, 1H), 4.08 (m, 5H), 3.98-3.94 (m, 1H), 3.83-3.78 (m, 1H), 3.44-3.41 (m, 1H), 3.01-2.96 (m, 2H), 2.53-2.49 (m, 1 H), 2.42-2.33 (m, 3H), 2.23-2.16 (m, 1H), 1.94-1.84 (m, 1H).

### Example 150: N-((8-(2,2′-Dichloro-3′-(6-methoxy-5-(((((S)-5-oxopyrrolidin-2-yl)methyl)amino)methyl)-pyridin-2-yl)-[1,1′-biphenyl]-3-yl)-4-oxo-4H-pyrido[1,2-a]pyrimidin-3-yl)methyl)-O-methyl-D-serine

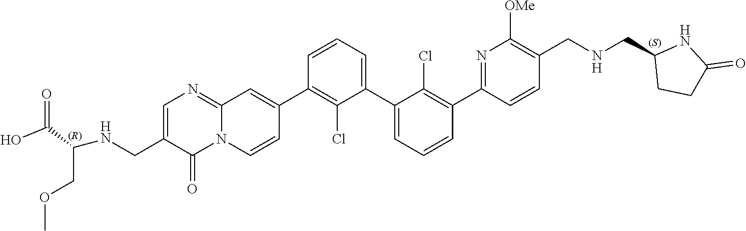

To a solution of tert-butyl N-[[6-[2-chloro-3-[2-chloro-3-(3-formyl-4-oxo-pyrido[1,2-a]-pyrimidin-8-yl)phenyl]phenyl]-2-methoxy-3-pyridyl]methyl]-N-[[(2S)-5-oxopyrrolidin-2-yl]-methyl]carbamate (50 mg, 0.07 mmol) and methyl (2R)-2-amino-3-methoxy-propanoate hydrochloride (45 mg, 0.26 mmol) in dioxane (5 mL), acetic acid (20 mg, 0.32 mmol) was added and stirred at 60° C. for 2 hrs and then added sodium triacetoxyborohydride (150 mg, 0.7 mmol) and stirred at room temperature for 2 hrs. Reaction mixture was diluted with water (10 mL), extracted with dichloromethane (2×20 mL), combined organic layer was washed with brine (30 mL), dried over sodium sulfate (10 g), solvents evaporated to give methyl (2R)-2-[[8-[3-[3-[5-[[tert-butoxycarbonyl-[[(2S)-5-oxo-pyrrolidin-2-yl]methyl]amino]methyl]-6-methoxy-2-pyridyl]-2-chloro-phenyl]-2-chloro-phenyl]-4-oxo-pyrido[1,2-a]pyrimidin-3-yl]methylamino]-3-methoxy-propanoate as an orange solid and used for next reaction without further purification (43 mg, 72.6% yield), m/z: 846 [M+H]⁺ observed.

To the methyl (2R)-2-[[8-[3-[3-[5-[[tert-butoxycarbonyl-[[(2S)-5-oxo-pyrrolidin-2-yl]methyl]-amino]methyl]-6-methoxy-2-pyridyl]-2-chloro-phenyl]-2-chloro-phenyl]-4-oxo-pyrido[1,2-a]-pyrimidin-3-yl]methylamino]-3-methoxy-propanoate (43 mg, 0.05 mmol) in dioxane (4 mL), HCl in dioxane (1 mL, 4 mmol, 4 M HCl in dioxane) and methanol (1 mL) were added and stirred for 1 hour at room temperature. Solvents were evaporated and the residue was dissolved in dioxane (3 mL) and aqueous lithium hydroxide solution (48 mg in 1 ml water, 2 mmol) was added and stirred at room temperature for 4 hours. Neutralized the reaction mixture with aqueous HCl solution, solvents evaporated and crude product was purified by HPLC with eluants 0 to 100% acetonitrile in water (formic acid as modifier) afforded N-((8-(2,2′-dichloro-3′-(6-methoxy-5-(((((S)-5-oxopyrrolidin-2-yl)methyl)-amino)methyl)-pyridin-2-yl)-[1,1′-biphenyl]-3-yl)-4-oxo-4H-pyrido-[1,2-a]pyrimidin-3-yl)methyl)-O-methyl-D-serine (as bis-formic acid salt) as a white solid (7.5 mg, 20.2% yield). m/z found: 732 [M+H]⁺. ¹H NMR (400 MHz, Methanol-d₄) δ9.14 (d, J=7.2 Hz, 1H), 8.50 (brs, 1H), 8.40 (brs, 1H), 7.82 (brs, 2H), 7.66-7.49 (m, 6H), 7.41-7.39 (d, J=7.2 Hz, 1H), 7.30 (brs, 1H), 4.35 (s, 2H), 4.10-3.83 (m, 9H), 3.40 (s, 3H), 3.31-3.29 (m, 2H), 3.01 (brs, 2H), 2.35 (brs, 3H), 1.86 (s, 1H).

### Example 151: ((8-(2,2′-Dichloro-3′-(6-methoxy-5-(((((S)-5-oxopyrrolidin-2-yl)methyl)amino)methyl)pyridin-2-yl)-[1,1′-biphenyl]-3-yl)-4-oxo-4H-pyrido[1,2-a]pyrimidin-3-yl)methyl)-L-serine

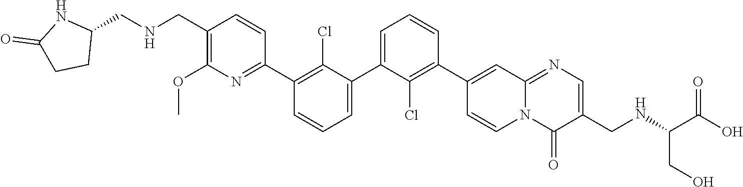 [SMILES: COc1nc(-c2cccc(-c3cccc(-c4ccn5c(=O)c(CN[C@@H](CO)C(=O)O)cnc5c4)c3Cl)c2Cl)ccc1CNC[C@@H]1CCC(=O)N1]

Methyl (2S)-2-[[8-[2-chloro-3-[2-chloro-3-[6-methoxy-5-[[[(2S)-5-oxopyrrolidin-2-yl]methylamino]methyl]-2-pyridyl]phenyl]phenyl]-4-oxo-pyrido[1,2-a]pyrimidin-3-yl]methylamino]-3-hydroxy-propanoate, (46 mg, 0.06 mmol) was dissolved in 0.5 ml THE and hydroxylithium;hydrate, (8 mg, 0.19 mmol) in 0.1 ml water was added. The resulting mixture was stirred at rt for 1 hour. The reaction was concentrated and the crude sample was purified by reverse phase HPLC to afford (2S)-2-[[8-[2-chloro-3-[2-chloro-3-[6-methoxy-5-[[[(2S)-5-oxopyrrolidin-2-yl]methylamino]methyl]-2-pyridyl]phenyl]phenyl]-4-oxo-pyrido[1,2-a]pyrimidin-3-yl]methylamino]-3-hydroxy-propanoic acid (10 mg, 21% yield) as a white fluffy solid (bis formic acid salt). MS: m/z found 717.4, 719 [M+H]⁺. ¹H NMR (400 MHz, Methanol-d₄) δ 9.16 (d, J=7.3 Hz, 1H), 8.51 (s, 1H), 8.47 (s, 2H), 7.83 (s, 1H), 7.78 (d, J=7.5 Hz, 1H), 7.66 (d, J=8.3 Hz, 1H), 7.61 (d, J=7.6 Hz, 3H), 7.54-7.48 (m, 2H), 7.42-7.36 (m, 1H), 7.27 (d, J=7.5 Hz, 1H), 4.38 (s, 2H), 4.09-4.05 (m, 1H), 4.03 (s, 3H), 3.97 (d, J=3.3 Hz, 2H), 3.91 (dd, J=11.7, 6.5 Hz, 3H), 3.73-3.69 (m, 1H), 2.85 (t, J=6.5 Hz, 2H), 2.34 (td, J=13.2, 4.9 Hz, 3H), 1.84 (dd, J=12.2, 7.8 Hz, 1H).

### Example 152: 8-(2,2′-Dichloro-3′-(6-methoxy-5-(((((S)-5-oxopyrrolidin-2-yl)methyl)amino)methyl)pyridin-2-yl)-[1,1′-biphenyl]-3-yl)-3-((6-oxohexahydropyrrolo[1,2-a]pyrazin-2(1H)-yl)methyl)-4H-pyrido[1,2-a]pyrimidin-4-one

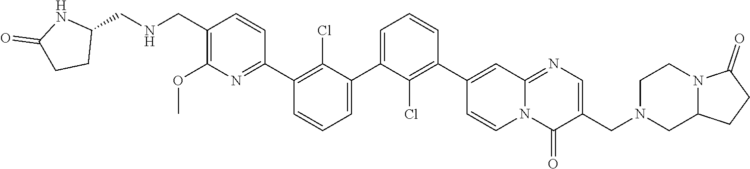 [SMILES: COc1nc(-c2cccc(-c3cccc(-c4ccn5c(=O)c(CN6CCN7C(=O)CCC7C6)cnc5c4)c3Cl)c2Cl)ccc1CNC[C@@H]1CCC(=O)N1]

2,3,4,7,8,8a-Hexahydro-1H-pyrrolo[1,2-a]pyrazin-6-one (14.43 mg, 0.10 mmol), tert-butyl N-[[6-[2-chloro-3-[2-chloro-3-(3-formyl-4-oxo-pyrido[1,2-a]pyrimidin-8-yl)phenyl]phenyl]-2-methoxy-3-pyridyl]methyl]-N-[[(2S)-5-oxopyrrolidin-2-yl]methyl]carbamate (30 mg, 0.04 mmol) and acetic acid (5 mg, 0.08 mmol) were dissolved in 1 ml of 1:1 MeOH/THF solvent mix, and the reaction was stirred at 25° C. for 3 hours. NaBH₃CN (5 mg, 0.08 mmol) was added portion-wise, and the resulting mixture was stirred at 25° C. for 1 hour. The reaction was concentrated and the sample was dissolved in water (3 mL), then extracted with DCM (3×2 ml). The combined organics were further washed with brine, dried over sodium sulfate, then filtered and concentrated to afford tert-butyl N-[[6-[2-chloro-3-[2-chloro-3-[4-oxo-3-[(6-oxo-1,3,4,7,8,8a-hexahydropyrrolo[1,2-a]pyrazin-2-yl)methyl]pyrido[1,2-a]pyrimidin-8-yl]phenyl]phenyl]-2-methoxy-3-pyridyl]methyl]-N-[[(2S)-5-oxopyrrolidin-2-yl]methyl]carbamate (35 mg) as a yellow foam. Material was used as—is for the next step. MS: m/z found 852, 854[M+H]⁺.

tert-butyl N-[[6-[2-chloro-3-[2-chloro-3-[4-oxo-3-[(6-oxo-1,3,4,7,8,8a-hexahydropyrrolo[1,2-a]pyrazin-2-yl)methyl]pyrido[1,2-a]pyrimidin-8-yl]phenyl]phenyl]-2-methoxy-3-pyridyl]methyl]-N-[[(2S)-5-oxopyrrolidin-2-yl]methyl]carbamate (35 mg, 0.04 mmol) was dissolved in 0.5 ml DCM and 4M HCl solution in dioxane (41 μL, 6 mg, 0.16 mmol) was added. The reaction was stirred at rt for 20 minutes and the mixture was subsequently concentrated. Crude sample was purified by reverse phase HPLC to afford 8-[2-chloro-3-[2-chloro-3-[6-methoxy-5-[[[(2S)-5-oxopyrrolidin-2-yl]methylamino]methyl]-2-pyridyl]phenyl]phenyl]-3-[(6-oxo-1,3,4,7,8,8a-hexahydropyrrolo[1,2-a]pyrazin-2-yl)methyl]pyrido[1,2-a]pyrimidin-4-one (18.9 mg, 61.2% yield) (bis formic acid salt). MS: m/z found 752.4, 754 [M+H]⁺. ¹H NMR (400 MHz, Methanol-d₄) δ 9.13 (d, J=7.4 Hz, 1H), 8.44 (s, 1H), 7.78 (d, J=7.6 Hz, 2H), 7.68-7.56 (m, 3H), 7.56-7.48 (m, 3H), 7.40 (dd, J=7.5, 1.8 Hz, 1H), 7.28 (d, J=7.4 Hz, 1H), 4.04 (s, 3H), 3.98 (d, J=3.3 Hz, 2H), 3.95-3.87 (m, 2H), 3.77-3.64 (m, 3H), 3.18-3.10 (m, 1H), 3.03-2.82 (m, 4H), 2.44-2.29 (m, 5H), 2.24-2.08 (m, 2H), 1.94 (t, J=10.9 Hz, 1H), 1.89-1.78 (m, 1H), 1.71-1.57 (m, 1H).

### Example 153: 8-(2,2′-Dichloro-3′-(6-methoxy-5-(((((S)-5-oxopyrrolidin-2-yl)methyl)amino)methyl)pyridin-2-yl)-[1,1′-biphenyl]-3-yl)-3-((2-(hydroxymethyl)pyrrolidin-1-yl)methyl)-4H-pyrido[1,2-a]pyrimidin-4-one

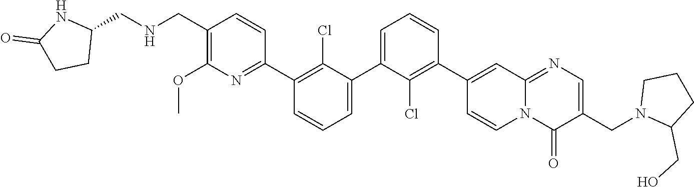 [SMILES: COc1nc(-c2cccc(-c3cccc(-c4ccn5c(=O)c(CN6CCCC6CO)cnc5c4)c3Cl)c2Cl)ccc1CNC[C@@H]1CCC(=O)N1]

tert-Butyl N-[[6-[2-chloro-3-[2-chloro-3-(3-formyl-4-oxo-pyrido[1,2-a]pyrimidin-8-yl)phenyl]phenyl]-2-methoxy-3-pyridyl]methyl]-N-[[(2S)-5-oxopyrrolidin-2-yl]methyl]carbamate (35 mg, 0.05 mmol), pyrrolidin-2-ylmethanol (12 mg, 0.12 mmol) and acetic acid (6 mg, 0.10 mmol) were dissolved in 1 ml of 1:1 MeOH/THF solvent mix, and the reaction was stirred at 25° C. for 3 hours. NaBH₃CN (6 mg, 0.10 mmol) was added portion-wise, and the resulting mixture was stirred at 25° C. for 1 hour. The reaction was concentrated and the sample was dissolved in water (3 mL), then extracted with DCM (3×2 mL). The combined organics were further washed with brine, dried over sodium sulfate, then filtered and concentrated to afford tert-butyl N-[[6-[2-chloro-3-[2-chloro-3-[3-[[2-(hydroxymethyl)pyrrolidin-1-yl]methyl]-4-oxo-pyrido[1,2-a]pyrimidin-8-yl]phenyl]phenyl]-2-methoxy-3-pyridyl]methyl]-N-[[(2S)-5-oxopyrrolidin-2-yl]methyl]carbamate (39 mg) as a yellow foam. Material was used as—is for below. MS: m/z found 813, 815 [M+H]⁺.

tert-butyl N-[[6-[2-chloro-3-[2-chloro-3-[3-[[2-(hydroxymethyl)pyrrolidin-1-yl]methyl]-4-oxo-pyrido[1,2-a]pyrimidin-8-yl]phenyl]phenyl]-2-methoxy-3-pyridyl]methyl]-N-[[(2S)-5-oxopyrrolidin-2-yl]methyl]carbamate (39 mg, 0.05 mmol) was dissolved in 0.5 ml DCM and 4M HCl solution in dioxane (48 μL, 7 mg, 0.19 mmol) was added. The reaction was stirred at rt for 20 minutes and the mixture was subsequently concentrated. Crude sample was purified by reverse phase HPLC to afford 8-[2-chloro-3-[2-chloro-3-[6-methoxy-5-[[[(2S)-5-oxopyrrolidin-2-yl]methylamino]methyl]-2-pyridyl]phenyl]phenyl]-3-[[2-(hydroxymethyl)pyrrolidin-1-yl]methyl]pyrido[1,2-a]pyrimidin-4-one (16 mg, 47% yield) as a white fluffy solid (bis formic acid salt). MS: m/z found 713.35, 715 [M+H]⁺. ¹H NMR (400 MHz, Methanol-d₄) δ 9.17 (d, J=7.3 Hz, 1H), 8.51 (d, J=15.9 Hz, 2H), 7.82 (d, J=1.9 Hz, 1H), 7.74 (d, J=7.4 Hz, 1H), 7.69-7.57 (m, 4H), 7.55-7.48 (m, 2H), 7.42-7.35 (m, 1H), 7.29-7.21 (m, 1H), 4.42 (d, J=13.4 Hz, 1H), 4.02 (d, J=3.6 Hz, 4H), 3.91-3.79 (m, 4H), 3.68 (dd, J=11.9, 5.2 Hz, 1H), 2.93 (s, 1H), 2.71 (t, J=7.0 Hz, 2H), 2.45-2.22 (m, 3H), 2.13 (s, 1H), 1.93 (d, J=80.3 Hz, 4H).

### Example 154: (S)-3-(((8-(2,2′-Dichloro-3′-(6-methoxy-5-((((5-oxopyrrolidin-2-yl)methyl)amino)-methyl)pyridin-2-yl)-[1,1′-biphenyl]-3-yl)-4-oxo-4H-pyrido[1,2-a]pyrimidin-3-yl)methyl)amino)-3-methylbutanoic acid

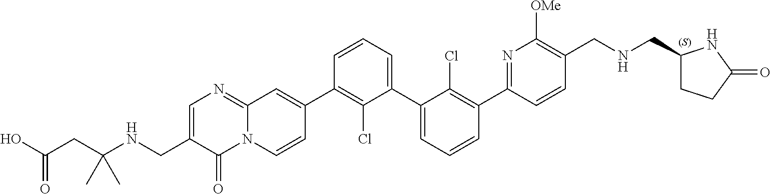

To a solution of tert-butyl N-[[6-[2-chloro-3-[2-chloro-3-(3-formyl-4-oxo-pyrido[1,2-a]-pyrimidin-8-yl)phenyl]phenyl]-2-methoxy-3-pyridyl]methyl]-N-[[(2S)-5-oxopyrrolidin-2-yl]-methyl]carbamate (50 mg, 0.07 mmol) and methyl 3-amino-3-methyl-butanoate (45 mg, 0.35 mmol) in dioxane (3 mL), acetic acid (10 mg, 0.16 mmol) was added and stirred at 60° C. for 3 h and then added sodium triacetoxyborohydride (45 mg, 0.21 mmol) and stirred for 1 hrs at room temperature. Reaction mixture was diluted with water (10 mL), extracted with dichloromethane (2×20 mL), combined organic layer was washed with brine (30 mL), dried over sodium sulfate (10 g), solvents evaporated to give methyl 3-[[8-[3-[3-[5-[[tert-butoxycarbonyl-[[(2S)-5-oxopyrrolidin-2-yl]methyl]amino]methyl]-6-methoxy-2-pyridyl]-2-chloro-phenyl]-2-chloro-phenyl]-4-oxo-pyrido[1,2-a]pyrimidin-3-yl]methylamino]-3-methyl-butanoate (43 mg) as an orange solid and used for next reaction without further purification (43 mg, 72.9% yield), m/z: 844 [M+H]⁺ observed.

To a solution of methyl 3-[[8-[3-[3-[5-[[tert-butoxycarbonyl-[[(2S)-5-oxopyrrolidin-2-yl]-methyl]amino]methyl]-6-methoxy-2-pyridyl]-2-chloro-phenyl]-2-chloro-phenyl]-4-oxo-pyrido-[1,2-a]pyrimidin-3-yl]methylamino]-3-methyl-butanoate (43 mg, 0.05 mmol) in dioxane (4 mL), HCl in Dioxane (1 mL, 4 mmol, 4 M HCl in dioxane) and methanol (1 mL) was added and stirred for 1 hour at room temperature. Solvents evaporated and the residue was dissolved in dioxane (3 mL) and aqueous lithium hydroxide solution (48 mg in 1 mL water, 2 mmol) was added and stirred at room temperature for 4 hours. Neutralized the reaction mixture with aqueous HCl solution, solvents evaporated and crude product was purified by HPLC with eluants 0 to 100% acetonitrile in water (formic acid as modifier) furnished (S)-3-(((8-(2,2′-dichloro-3′-(6-methoxy-5-((((5-oxopyrrolidin-2-yl)methyl)-amino)-methyl)pyridin-2-yl)-[1,1′-biphenyl]-3-yl)-4-oxo-4H-pyrido-[1,2-a]pyrimidin-3-yl)-methyl)amino)-3-methylbutanoic acid (as bis-formic acid salt) as a white solid (12 mg, 32.3% yield). m/z: 730 [M+H]+ observed. ¹H NMR (400 MHz, Methanol-d₄) δ 9.11 (d, J=7.6 Hz, 1H), 8.52 (s, 1H), 8.32 (brs, 1H), 7.88 (d, J=7.2 Hz, 1H), 7.79 (s, 1H), 7.65 (d, J=7.2 Hz, 1H), 7.59-7.47 (m, 5H), 7.38 (d, J=7.6 Hz, 1H), 7.30 (d, J=7.2 Hz, 1H), 4.25-4.22 (m, 4H), 4.05-4.02 (m, 4H), 3.17 (brs, 2H), 2.55 (brs, 2H), 2.45-2.31 (m, 3H), 1.90-1.87 (m, 1H), 1.50 (s, 6H).

### Example 155: (S)-5-((6-(2,2′-Dichloro-3′-(6-methoxy-5-(((((S)-5-oxopyrrolidin-2-yl)methyl)amino)-methyl)pyridin-2-yl)-[1,1′-biphenyl]-3-yl)-8-methoxy-3,4-dihydroisoquinolin-2(1H)-yl)methyl)pyrrolidin-2-one

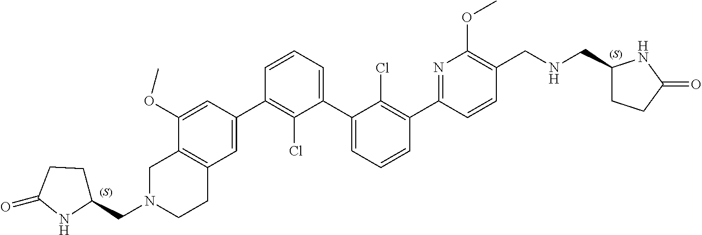

### (S)-5-((6-Bromo-8-methoxy-3,4-dihydroisoquinolin-2(1H)-yl)methyl)pyrrolidin-2-one

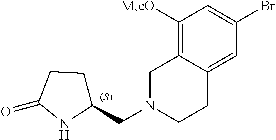

To a solution of 6-bromo-8-methoxy-1,2,3,4-tetrahydroisoquinoline (245 mg, 1.01 mmol) in DMF (5 mL) at room temperature, sodium hydride (50 mg, 60% in oil dispersion, 2 mmol) was added and stirred for 30 min, and then (5S)-5-(bromomethyl)pyrrolidin-2-one (180 mg, 1.01 mmol) was added and stirred at room temperature for 2 h. Reaction mixture was poured into a saturated aqueous sodium chloride solution (50 mL), extracted with dichloromethane (2×20 mL), organic layer separated and dried over sodium sulfate (10 g) and solvent evaporated. Crude product was subjected to flash chromatography over silica gel using eluants 0 to 10% methanol in dichloromethane furnished (S)-5-((6-bromo-8-methoxy-3,4-dihydroisoquinolin-2(1H)-yl)methyl)pyrrolidin-2-one as a white solid (170 mg, 50% yield). m/z: 340 [M+H]⁺ observed. ¹H NMR (400 MHz, CDCl₃) δ 6.95 (brs, 1H), 6.92 (brs, 1H), 6.10 (brs, 1H), 4.10-4.00 (m, 2H), 3.87 (s, 3H), 3.73-3.69 (m, 2H), 3.54-3.39 (m, 3H), 2.95-2.82 (m, 2H), 2.64-2.43 (m, 3H), 2.00-1.92 (m, 1H).

### (S)-6-(2,2′-Dichloro-3′-(8-methoxy-2-((5-oxopyrrolidin-2-yl)methyl)-1,2,3,4-tetrahydro-isoquinolin-6-yl)-[1,1′-biphenyl]-3-yl)-2-methoxynicotinaldehyde

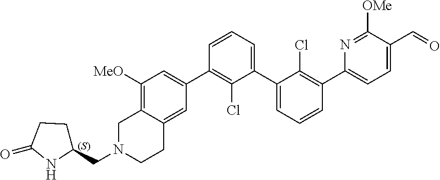

To a mixture of 6-(2,2′-dichloro-3′-(4,4,5,5-tetramethyl-1,3,2-dioxaborolan-2-yl)-[1,1′-biphenyl]-3-yl)-2-methoxynicotinaldehyde (121 mg, 0.25 mmol) and (S)-5-((6-bromo-8-methoxy-3,4-dihydroisoquinolin-2(1H)-yl)methyl)pyrrolidin-2-one (102 mg, 0.3 mmol), 1,1′-bis(diphenylphosphino)ferrocene-palladium(II) dichloride dichloromethane complex (10 mg, 0.0012 mmol) and potassium carbonate (70 mg, 0.5 mmol) in dioxane/water (10 ml, 9:1), nitrogen gas was bubbled through the solution for 2 minutes, and then reaction vessel was sealed and heated at 105° C. for 3 hours. The reaction was cooled to room temperature and the mixture was diluted with 20 mL water and extracted with EtOAc (3×20 ml). The combined organics were concentrated, and the crude product was subjected to flash column chromatography over silica gel using eluants 0 to 10% methanol in dichloromethane furnished (S)-6-(2,2′-dichloro-3′-(8-methoxy-2-((5-oxopyrrolidin-2-yl)methyl)-1,2,3,4-tetrahydroisoquinolin-6-yl)-[1,1′-biphenyl]-3-yl)-2-methoxy-nicotinaldehyde as an yellow powder (80 mg, 52% yield). m/z: 617 [M+H]+ observed ¹H NMR (400 MHz, Methanol-d₄) δ 10.30 (s, 1H), 8.56 (s, 1H), 7.80 (d, J=7.6 Hz, 1H), 7.67 (dd, J=7.6 Hz, J=1.6 Hz, 1H), 7.57-7.35 (m, 6H), 6.90 (d, J=3.6 Hz, 2H), 4.22 (s, 2H), 4.07 (s, 3H), 3.90-3.83 (m, 5H), 3.21-2.90 (m, 8H), 1.88-1.80 (m, 1H).

### (S)-5-((6-(2,2′-Dichloro-3′-(6-methoxy-5-(((((S)-5-oxopyrrolidin-2-yl)methyl)amino)-methyl)pyridin-2-yl)-[1,1′-biphenyl]-3-yl)-8-methoxy-3,4-dihydroisoquinolin-2(1H)-yl)methyl)pyrrolidin-2-one

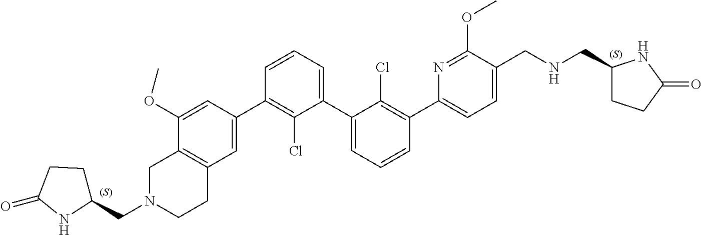

A solution of (S)-6-(2,2′-dichloro-3′-(8-methoxy-2-((5-oxopyrrolidin-2-yl)methyl)-1,2,3,4-tetrahydroisoquinolin-6-yl)-[1,1′-biphenyl]-3-yl)-2-methoxy-nicotinaldehyde (43 mg, 0.07 mmol), (5S)-5-(aminomethyl)pyrrolidin-2-one (25 mg, 0.2 mmol) and acetic acid (10 mg, 0.16 mmol) in dioxane/methanol (1:1, 4 mL) was stirred overnight at 60° C. and then added sodium triacetoxyborohydride (90 mg, 0.42 mmol) and stirred for 1 h at room temperature. Reaction mixture was diluted with water (10 mL), extracted with dichloromethane (2×20 mL), combined organic layer was washed with brine (30 mL), dried over sodium sulfate (10 g), solvents evaporated to give crude product as an orange solid and used for next reaction without further purification (45 mg, 78.5% yield), m/z found: 815 [M+H]⁺.

The orange solid product (45 mg, 0.055 mmol) was dissolved in dioxane (3 mL), HCl in dioxane (1 mL, 4 mmol, 4 M HCl in dioxane) was added and stirred for 1 hour at room temperature. Neutralized the reaction mixture with aqueous HCl solution, solvents evaporated and crude product was purified by HPLC with eluants 0 to 100% acetonitrile in water (formic acid as modifier) furnished (S)-5-((6-(2,2′-dichloro-3′-(6-methoxy-5-(((((S)-5-oxopyrrolidin-2-yl)-methyl)amino)-methyl)pyridin-2-yl)-[1,1′-biphenyl]-3-yl)-8-methoxy-3,4-dihydroisoquinolin-2(1H)-yl)methyl)pyrrolidin-2-one (as bis-formic acid salt) as a white solid (21 mg, 42.1% yield). m/z: 715 [M+H]+ observed. ¹H NMR (400 MHz, Methanol-d₄) δ 8.35 (brs, 2H), 7.85 (d, J=7.6 Hz, 1H), 7.63 (dd, J=7.6 Hz, J=1.6 Hz, 1H), 7.49-7.28 (m, 6H), 6.83 (d, J=3.6 Hz, 2H), 4.22 (brs, 2H), 4.11-4.01 (m, 5H), 3.94-3.77 (m, 5H), 3.18-2.78 (m, 9H), 2.42-2.29 (m, 6H), 1.92-1.80 (m, 2H).

### Example 156: 8-(2,2′-Dichloro-3′-(6-methoxy-5-(((((S)-5-oxopyrrolidin-2-yl)methyl)amino)methyl)pyridin-2-yl)-[1,1′-biphenyl]-3-yl)-2-(((((S)-5-oxopyrrolidin-2-yl)methyl)amino)methyl)-4H-pyrido[1,2-a]pyrimidin-4-one

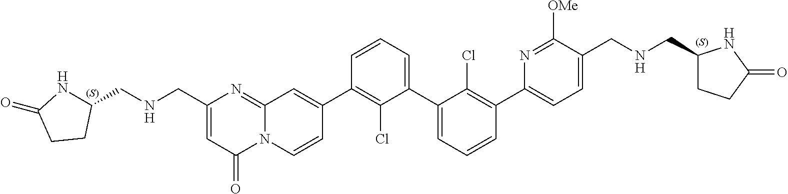

### (S)-((8-Bromo-4-oxo-4H-pyrido[1,2-a]pyrimidin-2-yl)methyl)((5-oxopyrrolidin-2-yl)methyl)-carbamate

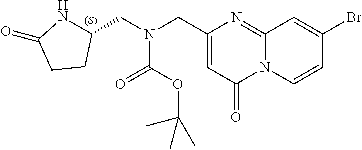

To a mixture of 8-bromo-2-(chloromethyl)pyrido[1,2-a]pyrimidin-4-one (275 mg, 1.01 mmol), (5S)-5-(aminomethyl)pyrrolidin-2-one (150 mg, 1.31 mmol) and DIEA (0.34 ml, 2 mmol) in anhydrous dioxane (10 mL) were stirred at 100° C. for overnight. Reaction mixture was poured into aqueous saturated sodium chloride solution (50 mL), extracted with dichloromethane (2×20 mL), organic layer was separated and dried over sodium sulfate (10 g) and solvent evaporated. Crude product was subjected to flash column chromatography over silica gel using eluents 0 to 10% methanol in dichloromethane furnished 120 mg (34% yield) of 7-bromo-2-[[[(2S)-5-oxopyrrolidin-2-yl]methylamino]methyl] pyrido[1,2-a]pyrimidin-4-one as a light brown solid.

The 7-bromo-2-[[[(2S)-5-oxopyrrolidin-2-yl]methylamino]methyl] pyrido[1,2-a]pyrimidin-4-one (120 mg, 0.35 mmol) and tert-butoxycarbonyl tert-butyl carbonate (155 mg, 0.7 mmol) in anhydrous THE (10 mL), DIEA (0.34 mL, 2 mmol) was added and stirred overnight at room temperature. Reaction mixture was diluted with saturated aqueous sodium bicarbonate (50 mL), extracted with ethyl acetate (2×25 mL), combined organic layer was washed with brine (30 mL), dried over sodium sulfate (15 g), and solvents removed to give crude product. Crude product was subjected to flash column chromatography over silica gel using eluents 0 to 10% methanol in dichloromethane furnished tert-butyl (S)-((8-bromo-4-oxo-4H-pyrido[1,2-a]pyrimidin-2-yl)methyl)((5-oxopyrrolidin-2-yl)methyl)-carbamate as a white solid (150 mg, 95% yield). m/z: 452 [M+H]⁺ observed. ¹H NMR (400 MHz, CDCl₃) δ 9.26 (d, J=2.0 Hz, 1H), 7.91-7.80 (m, 3H), 6.42 (s, 1H), 4.54 (d, J=16.0 Hz, 1H), 4.23 (d, J=16.0 Hz, 1H), 4.20-4.15 (s, 1H), 3.87 (dd, J=14 Hz, J=2.8 Hz, 1H), 3.10 (dd, J=14 Hz, J=9.6 Hz, 1H), 2.47-2.43 (m, 2H), 1.80-1.77 (m, 2H), 1.41 (m, 9H).

### tert-Butyl (S)-((8-(2,2′-dichloro-3′-(5-formyl-6-methoxypyridin-2-yl)-[1,1′-biphenyl]-3-yl)-4-oxo-4H-pyrido[1,2-a]pyrimidin-2-yl)methyl)((5-oxopyrrolidin-2-yl)methyl)carbamate

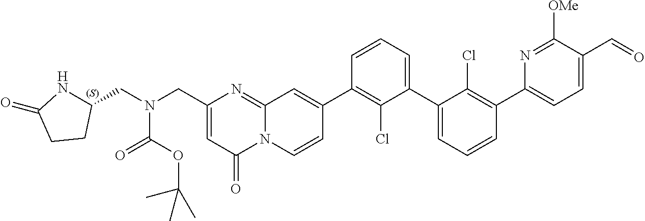

To a mixture of 6-(2,2′-dichloro-3′-(4,4,5,5-tetramethyl-1,3,2-dioxaborolan-2-yl)-[1,1′-biphenyl]-3-yl)-2-methoxynicotinaldehyde (121 mg, 0.25 mmol) and tert-butyl (S)-((8-bromo-4-oxo-4H-pyrido[1,2-a]pyrimidin-2-yl)methyl)((5-oxopyrrolidin-2-yl)methyl)-carbamate (160 mg, 0.35 mmol), 1,1′-bis(diphenylphosphino)ferrocene-palladium(ii) dichloride dichloromethane complex (10 mg, 0.0012 mmol) and potassium carbonate (70 mg, 0.5 mmol) in dioxane/water (10 ml, 9:1), nitrogen gas was bubbled through the solution for 2 minutes, then the vessel was sealed and heated at 105° C. for 3 hours. The reaction was cooled to room temperature and the mixture was diluted with 10 ml water and extracted with ethyl acetate (3×10 ml). The combined organics were concentrated, and the crude product was subjected to flash column chromatography over silica gel using eluants 0 to 10% methanol in dichloromethane furnished tert-butyl (S)-((8-(2,2′-dichloro-3′-(5-formyl-6-methoxypyridin-2-yl)-[1,1′-biphenyl]-3-yl)-4-oxo-4H-pyrido[1,2-a]pyrimidin-2-yl)-methyl)((5-oxopyrrolidin-2-yl)methyl)carbamate as an yellow powder (55 mg, 35% yield). m/z: 729 [M+H]+ observed ¹H NMR (400 MHz, Methanol-d₄) δ 10.30 (s, 1H), 9.26 (d, J=7.6 Hz, 1H), 8.21 (d, J=7.6 Hz, 1H), 7.79-7.34 (m, 9H), 6.42 (s, 1H), 4.52 (d, J=16.0 Hz, 1H), 4.30 (s, 3H), 4.20 (d, J=16.0 Hz, 1H), 4.15-4.10 (s, 1H), 3.89 (dd, J=14 Hz, J=2.8 Hz, 1H), 3.12 (dd, J=14 Hz, J=9.6 Hz, 1H), 2.44-2.41 (m, 2H), 2.31-2.26 (m, 1H), 1.91-1.87 (m, 1H), 1.55 (brs, 9H).

### 8-(2,2′-Dichloro-3′-(6-methoxy-5-(((((S)-5-oxopyrrolidin-2-yl)methyl)amino)methyl)-pyridin-2-yl)-[1,1′-biphenyl]-3-yl)-2-(((((S)-5-oxopyrrolidin-2-yl)methyl)amino)methyl)-4H-pyrido[1,2-a]pyrimidin-4-one

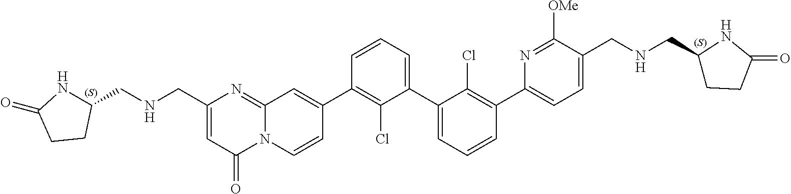

A solution of tert-butyl (S)-((8-(2,2′-dichloro-3′-(5-formyl-6-methoxypyridin-2-yl)-[1,1′-biphenyl]-3-yl)-4-oxo-4H-pyrido[1,2-a]pyrimidin-2-yl)methyl)((5-oxopyrrolidin-2-yl)methyl)-carbamate (40 mg, 0.055 mmol), (5S)-5-(aminomethyl)pyrrolidin-2-one (25 mg, 0.2 mmol) and acetic acid (10 mg, 0.16 mmol) in dioxane/methanol (1:1, 3 mL) was stirred overnight at 60° C. and then added sodium triacetoxyborohydride (90 mg, 0.42 mmol) and stirred for 1 h at room temperature. Reaction mixture was diluted with water (10 mL), extracted with dichloromethane (2×20 mL), combined organic layer was washed with brine (30 mL), dried over sodium sulfate (10 g), solvents evaporated to give crude product (45 mg) as an orange solid and used for next reaction without further purification.

The orange solid product (45 mg, 0.05 mmol) was dissolved in dioxane (3 mL), HCl in Dioxane (1 mL, 4 mmol, 4 M HCl in dioxane) was added and stirred for 1 hour at room temperature. Neutralized the reaction mixture with aqueous HCl solution, solvents evaporated and crude product was purified by HPLC with eluants 0 to 100% acetonitrile in water (formic acid as modifier) furnished 8-(2,2′-dichloro-3′-(6-methoxy-5-(((((S)-5-oxopyrrolidin-2-yl)methyl)amino)methyl)-pyridin-2-yl)-[1,1′-biphenyl]-3-yl)-2-(((((S)-5-oxo-pyrrolidin-2-yl)methyl)amino)methyl)-4H-pyrido[1,2-a]pyrimidin-4-one (as bis-formic acid salt) as a white solid (13 mg, 32.6% yield), m/z found: 727 [M+H]⁺. ¹H NMR (400 MHz, Methanol-d₄) δ 9.09 (d, J=1.2 Hz, 1H), 8.38 (brs, 1H), 8.13 (dd, J=9.2 Hz, J=1.2 Hz, 1H), 7.84 (d, J=8.0 Hz, 1H), 7.79 (d, J=9.2 Hz, 1H), 7.66-7.30 (m, 7H), 6.57 (s, 2H), 4.17-3.95 (m, 9H), 3.11-2.91 (m, 6H), 2.42-2.31 (m, 6H), 1.93-1.85 (m, 2H).

### Example 157: (S)-8-(2,2′-Dichloro-3′-(5-((3-hydroxy-3-methylazetidin-1-yl)methyl)-6-methoxypyridin-2-yl)-[1,1′-biphenyl]-3-yl)-3-((((5-oxopyrrolidin-2-yl)methyl)amino)methyl)-4H-pyrido[1,2-a]pyrimidin-4-one

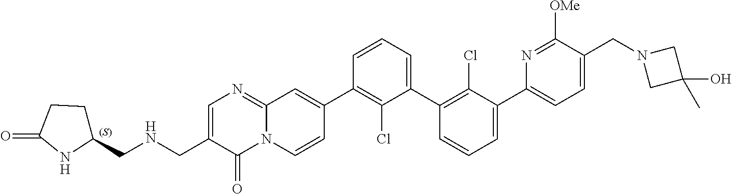

### tert-Butyl (S)-((8-(2,2′-dichloro-3′-(5-((3-hydroxy-3-methylazetidin-1-yl)methyl)-6-methoxypyridin-2-yl)-[1,1′-biphenyl]-3-yl)-4-oxo-4H-pyrido[1,2-a]pyrimidin-3-yl)methyl)((5-oxopyrrolidin-2-yl)methyl)carbamate

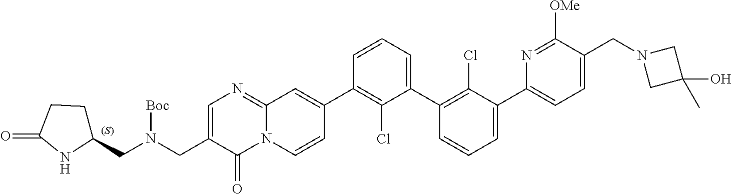

To a stirring solution of tert-butyl N-[[8-[2-chloro-3-[2-chloro-3-(5-formyl-6-methoxy-2-pyridyl)phenyl]phenyl]-4-oxo-pyrido[1,2-a]pyrimidin-3-yl]methyl]-N-[[rac-(2S)-5-oxopyrrolidin-2-yl]methyl]carbamate (85 mg, 0.09 mmol) in DCM/MeOH (2 mL, 1:1) at rt was added 3-methylazetidin-3-ol (41 mg, 0.47 mmol). The reaction was stirred for 15 minutes at rt and was treated sequentially with acetic acid (5.6 mg, 0.09 mmol), followed by sodium triacetoxyborohydride (59 mg, 0.28 mmol). After 2 h of stirring at rt, the solvent was removed under reduced pressure to afford crude tert-butyl (S)-((8-(2,2′-dichloro-3′-(5-((3-hydroxy-3-methylazetidin-1-yl)methyl)-6-methoxypyridin-2-yl)-[1,1′-biphenyl]-3-yl)-4-oxo-4H-pyrido[1,2-a]pyrimidin-3-yl)methyl)((5-oxopyrrolidin-2-yl)methyl)carbamate that was used in the following TFA deprotection without further purification. MS: m/z found 800.3 [M+H]⁺.

### (S)-8-(2,2′-Dichloro-3′-(5-((3-hydroxy-3-methylazetidin-1-yl)methyl)-6-methoxypyridin-2-yl)-[1,1′-biphenyl]-3-yl)-3-((((5-oxopyrrolidin-2-yl)methyl)amino)methyl)-4H-pyrido[1,2-a]pyrimidin-4-one

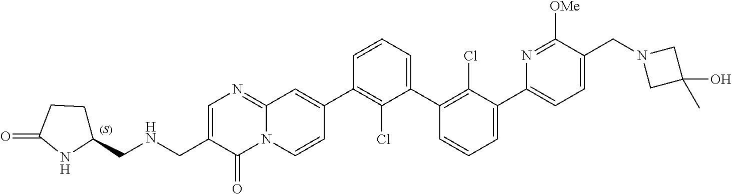

To a stirring solution of tert-butyl (S)-((8-(2,2′-dichloro-3′-(5-((3-hydroxy-3-methylazetidin-1-yl)methyl)-6-methoxypyridin-2-yl)-[1,1′-biphenyl]-3-yl)-4-oxo-4H-pyrido[1,2-a]pyrimidin-3-yl)methyl)((5-oxopyrrolidin-2-yl)methyl)carbamate (74 mg, 0.09 mmol) in dry DCM (1 mL) at rt was added trifluoroacetic acid (0.35 mL, 4.63 mmol) in a steady stream. The reaction was complete after 2 h of stirring at rt and the solvent and excess acid were removed under reduced pressure. The crude yellow oil was dissolved in 2 mL DMSO and purified by HPLC from 10-65% MeCN/H₂O over 10 min ramp to afford (S)-8-(2,2′-dichloro-3′-(5-((3-hydroxy-3-methylazetidin-1-yl)methyl)-6-methoxypyridin-2-yl)-[1,1′-biphenyl]-3-yl)-3-((((5-oxopyrrolidin-2-yl)methyl)amino)methyl)-4H-pyrido[1,2-a]pyrimidin-4-one (34.6 mg) as a white solid. MS: m/z found 701.2 [M+H]⁺. ¹H NMR (400 MHz, Methanol-d₄) δ 9.18 (d, J=7.4 Hz, 1H), 8.49 (s, 1H), 8.38 (s, 1H), 7.89-7.80 (m, 2H), 7.70-7.57 (m, 4H), 7.57-7.50 (m, 2H), 7.47-7.40 (m, 1H), 7.35 (dd, J=7.5, 1.1 Hz, 1H), 4.40 (s, 2H), 4.15 (d, J=4.3 Hz, 2H), 4.13-4.05 (m, 5H), 3.98 (d, J=10.2 Hz, 3H), 3.04 (dd, J=9.8, 6.1 Hz, 2H), 2.40-2.29 (m, 3H), 1.88 (d, J=7.3 Hz, 1H), 1.53 (s, 3H).

### Example 158: 8-(2,2′-Dichloro-3′-(5-((((1r,3r)-3-hydroxycyclobutyl)amino)methyl)-6-methoxypyridin-2-yl)-[1,1′-biphenyl]-3-yl)-3-(((((S)-5-oxopyrrolidin-2-yl)methyl)amino)methyl)-4H-pyrido[1,2-a]pyrimidin-4-one

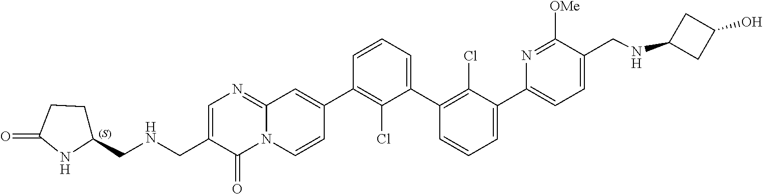

### tert-Butyl ((8-(2,2′-dichloro-3′-(5-((((1r,3r)-3-hydroxycyclobutyl)amino)methyl)-6-methoxypyridin-2-yl)-[1,1′-biphenyl]-3-yl)-4-oxo-4H-pyrido[1,2-a]pyrimidin-3-yl)methyl)(((S)-5-oxopyrrolidin-2-yl)methyl)carbamate

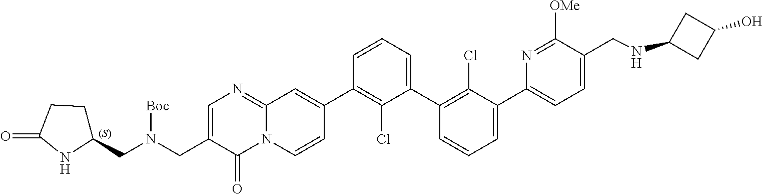

To a stirring solution of tert-butyl N-[[8-[2-chloro-3-[2-chloro-3-(5-formyl-6-methoxy-2-pyridyl)phenyl]phenyl]-4-oxo-pyrido[1,2-a]pyrimidin-3-yl]methyl]-N-[[(2S)-5-oxopyrrolidin-2-yl]methyl]carbamate (93 mg, 0.10 mmol) in DCM/MeOH (2 mL, 1:1) at rt was added 3-aminocyclobutanol (45 mg, 0.51 mmol). After 15 minutes of stirring at rt, the reaction was treated with acetic acid (6 mg, 0.10 mmol), followed by sodium triacetoxyborohydride (65 mg, 0.31 mmol) in one portion. After 2 h of stirring at rt, the solvent was removed under reduced pressure to afford crude tert-butyl ((8-(2,2′-dichloro-3′-(5-((((1r,3r)-3-hydroxycyclobutyl)amino)methyl)-6-methoxypyridin-2-yl)-[1,1′-biphenyl]-3-yl)-4-oxo-4H-pyrido[1,2-a]pyrimidin-3-yl)methyl)(((S)-5-oxopyrrolidin-2-yl)methyl)carbamate that was used in the following TFA deprotection without further purification. MS: m/z found 801.5 [M+H]⁺.

### 8-(2,2′-Dichloro-3′-(5-((((1r,3r)-3-hydroxycyclobutyl)amino)methyl)-6-methoxypyridin-2-yl)-[1,1′-biphenyl]-3-yl)-3-(((((S)-5-oxopyrrolidin-2-yl)methyl)amino)methyl)-4H-pyrido[1,2-a]pyrimidin-4-one

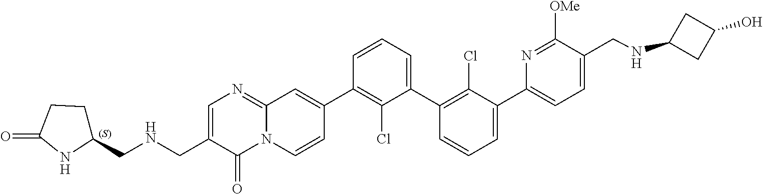

To a stirring solution of crude tert-butyl ((8-(2,2′-dichloro-3′-(5-((((1r,3r)-3-hydroxycyclobutyl)amino)methyl)-6-methoxypyridin-2-yl)-[1,1′-biphenyl]-3-yl)-4-oxo-4H-pyrido[1,2-a]pyrimidin-3-yl)methyl)(((S)-5-oxopyrrolidin-2-yl)methyl)carbamate (80 mg, 0.10 mmol) in dry DCM (1 mL) at rt was added trifluoroacetic acid (0.38 mL, 5.00 mmol) in a steady stream. The reaction was complete after 2 h of stirring at rt and the solvent and excess acid were removed under reduced pressure. The crude yellow oil was dissolved in DMSO (2 mL) and purified by HPLC from 10-65% MeCN/H₂O over 10 min ramp to afford 8-(2,2′-dichloro-3′-(5-((((1r,3r)-3-hydroxycyclobutyl)amino)methyl)-6-methoxypyridin-2-yl)-[1,1′-biphenyl]-3-yl)-3-(((((S)-5-oxopyrrolidin-2-yl)methyl)amino)methyl)-4H-pyrido[1,2-a]pyrimidin-4-one (13 mg) as a white solid. MS: m/z found 700.4 [M+H]⁺. ¹H NMR (400 MHz, Methanol-d₄) δ 9.16 (d, J=7.4 Hz, 1H), 8.53-8.39 (m, 3H), 7.90-7.84 (m, 1H), 7.81 (d, J=1.8 Hz, 1H), 7.70-7.51 (m, 6H), 7.47-7.41 (m, 1H), 7.35 (dd, J=7.5, 1.5 Hz, 1H), 4.52-4.45 (m, 1H), 4.15 (d, J=1.5 Hz, 2H), 4.08 (q, J=3.6, 2.6 Hz, 4H), 3.95 (d, J=7.2 Hz, 2H), 3.01-2.89 (m, 2H), 2.56-2.47 (m, 2H), 2.40-2.30 (m, 5H), 1.90-1.82 (m, 1H).

### Example 159: (S)-8-(3′-(5-((3,3-Bis(hydroxymethyl)azetidin-1-yl)methyl)-6-methoxypyridin-2-yl)-2,2′-dichloro-[1,1′-biphenyl]-3-yl)-3-((((5-oxopyrrolidin-2-yl)methyl)amino)methyl)-4H-pyrido[1,2-a]pyrimidin-4-one

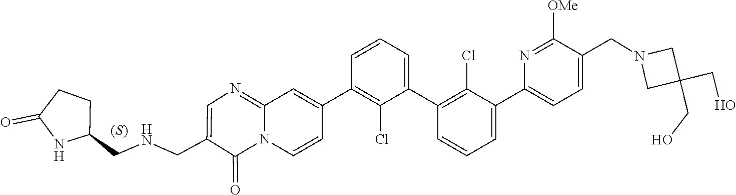

### tert-Butyl (S)-((8-(3′-(5-((2-oxa-6-azaspiro[3.3]heptan-6-yl)methyl)-6-methoxypyridin-2-yl)-2,2′-dichloro-[1,1′-biphenyl]-3-yl)-4-oxo-4H-pyrido[1,2-a]pyrimidin-3-yl)methyl)((5-oxopyrrolidin-2-yl)methyl)carbamate

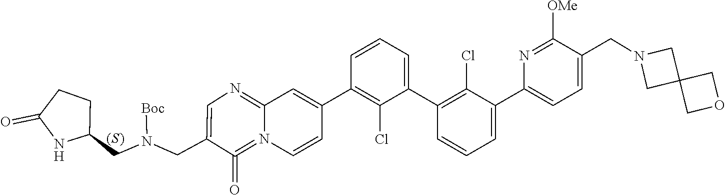

To a stirring solution of tert-butyl N-[[8-[2-chloro-3-[2-chloro-3-(5-formyl-6-methoxy-2-pyridyl)phenyl]phenyl]-4-oxo-pyrido[1,2-a]pyrimidin-3-yl]methyl]-N-[[(2S)-5-oxopyrrolidin-2-yl]methyl]carbamate (60 mg, 0.07 mmol) in a 1:1 mixture of DCM/MeOH (2 mL) was added 2-oxa-6-azaspiro[3.3]heptane (33 mg, 0.33 mmol). After stirring for 15 min at rt, the reaction was treated with acetic acid (4 mg, 0.07 mmol) and sodium triacetoxyborohydride (42 mg, 0.20 mmol) in one portion. After 2 h stirring at rt, the reaction was recharged with 2-oxa-6-azaspiro[3.3]heptane (33 mg, 0.33 mmol), followed by sodium triacetoxyborohydride (42 mg, 0.20 mmol) and the reaction was warmed to 50° C. while stirring for 16 h. The solvent was removed under reduced pressure to afford crude tert-butyl (S)-((8-(3′-(5-((2-oxa-6-azaspiro[3.3]heptan-6-yl)methyl)-6-methoxypyridin-2-yl)-2,2′-dichloro-[1,1′-biphenyl]-3-yl)-4-oxo-4H-pyrido[1,2-a]pyrimidin-3-yl)methyl)((5-oxopyrrolidin-2-yl)methyl)carbamate that was used in the following TFA deprotection without further purification. MS: m/z found 813.3 [M+H]⁺.

### (S)-8-(3′-(5-((3,3-Bis(hydroxymethyl)azetidin-1-yl)methyl)-6-methoxypyridin-2-yl)-2,2′-dichloro-[1,1′-biphenyl]-3-yl)-3-((((5-oxopyrrolidin-2-yl)methyl)amino)methyl)-4H-pyrido[1,2-a]pyrimidin-4-one

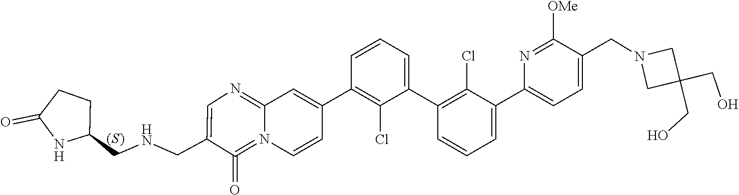

To a stirring solution of tert-butyl N-[[8-[2-chloro-3-[2-chloro-3-[6-methoxy-5-(2-oxa-6-azaspiro[3.3]heptan-6-ylmethyl)-2-pyridyl]phenyl]phenyl]-4-oxo-pyrido[1,2-a]pyrimidin-3-yl]methyl]-N-[[rac-(2S)-5-oxopyrrolidin-2-yl]methyl]carbamate (20 mg, 0.02 mmol) in DCM (1 mL) at rt was added trifluoroacetic acid (90 uL, 1.23 mmol) in a slow stream. The reaction was complete after 2 h of stirring at rt and the solvent and excess acid were removed under reduced pressure. The crude oil was dissolved in DMSO (2 mL) and purified by HPLC from 10-65% MeCN/H₂O over 10 min ramp to afford (S)-8-(3′-(5-((3,3-bis(hydroxymethyl)azetidin-1-yl)methyl)-6-methoxypyridin-2-yl)-2,2′-dichloro-[1,1′-biphenyl]-3-yl)-3-((((5-oxopyrrolidin-2-yl)methyl)amino)methyl)-4H-pyrido[1,2-a]pyrimidin-4-one (16.0 mg) as a white solid. MS: m/z found 749.2 [M+H]⁺. ¹H NMR (400 MHz, Methanol-d₄) δ 9.16 (d, J=7.3 Hz, 1H), 8.45 (d, J=8.5 Hz, 3H), 7.84-7.76 (m, 2H), 7.70-7.48 (m, 7H), 7.46-7.39 (m, 1H), 7.35-7.28 (m, 1H), 4.16 (s, 2H), 4.05 (t, J=2.2 Hz, 5H), 3.94 (s, 1H), 3.86 (d, J=1.7 Hz, 2H), 3.83-3.70 (m, 6H), 2.92 (dd, J=10.7, 6.2 Hz, 2H), 2.35 (s, 3H), 1.86 (s, 1H).

### Example 160: (S)-4-(((8-(2,2′-Dichloro-3′-(6-methoxy-5-((((5-oxopyrrolidin-2-yl)methyl)amino)methyl)pyridin-2-yl)-[1,1′-biphenyl]-3-yl)-4-oxo-4H-pyrido[1,2-a]pyrimidin-3-yl)methyl)amino)butanoic acid

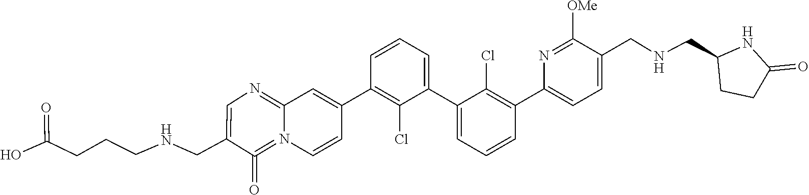

### (S)-4-(((8-(2,2′-Dichloro-3′-(6-methoxy-5-((((5-oxopyrrolidin-2-yl)methyl)amino)methyl)pyridin-2-yl)-[1,1′-biphenyl]-3-yl)-4-oxo-4H-pyrido[1,2-a]pyrimidin-3-yl)methyl)amino)butanoic acid

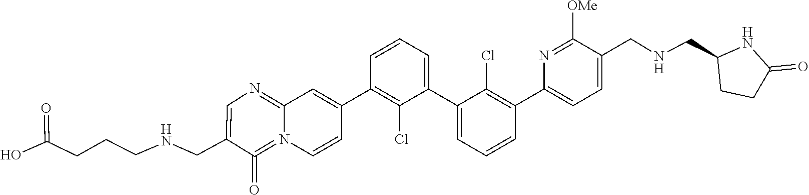

To a stirring suspension of methyl (S)-4-(((8-(2,2′-dichloro-3′-(6-methoxy-5-((((5-oxopyrrolidin-2yl)methyl)amino)methyl)pyridin-2-yl)-[1,1′-biphenyl]-3-yl)-4-oxo-4H-pyrido[1,2-a]pyrimidin-3-yl)methyl)amino)butanoate (15 mg, 0.02 mmol) in 0.5 ml THE at rt was added an aqueous solution of lithium hydroxide (3 mg, 0.06 mmol) in 0.1 mL water. The resulting mixture was stirred at rt for 1 h. The reaction was concentrated and the crude residue dissolved in 2 mL of DMSO was purified by reverse phase HPLC from 10-75% MeCN/H₂O over a ten-minute ramp to afford (S)-4-(((8-(2,2′-dichloro-3′-(6-methoxy-5-((((5-oxopyrrolidin-2-yl)methyl)amino)methyl)pyridin-2-yl)-[1,1′-biphenyl]-3-yl)-4-oxo-4H-pyrido[1,2-a]pyrimidin-3-yl)methyl)amino)butanoic acid (1.6 mg) as a white solid. MS: m/z found 715.3 [M+H]⁺. ¹H NMR (400 MHz, Methanol-d₄) δ 9.18 (dd, J=7.3, 2.6 Hz, 1H), 8.57-8.45 (m, 3H), 7.85 (d, J=2.4 Hz, 1H), 7.76 (dd, J=7.6, 2.6 Hz, 1H), 7.70-7.58 (m, 4H), 7.53 (dd, J=7.5, 2.7 Hz, 2H), 7.44-7.37 (m, 1H), 7.27 (dd, J=7.6, 2.6 Hz, 1H), 4.25 (d, J=2.6 Hz, 2H), 4.03 (d, J=2.7 Hz, 3H), 3.92 (d, J=3.3 Hz, 2H), 3.89 (s, 1H), 3.15 (dt, J=6.7, 4.0 Hz, 2H), 2.79 (d, J=7.9 Hz, 2H), 2.41 (d, J=6.2 Hz, 2H), 2.38-2.27 (m, 3H), 1.94 (s, 2H), 1.83 (d, J=11.7 Hz, 1H).

### Example 162: (S)-Methyl 1-((8-(2,2′-dichloro-3′-(6-methoxy-5-((((5-oxopyrrolidin-2-yl)methyl)amino)methyl)pyridin-2-yl)-[1,1′-biphenyl]-3-yl)-4-oxo-4H-pyrido[1,2-a]pyrimidin-3-yl)methyl)azetidine-3-carboxylate

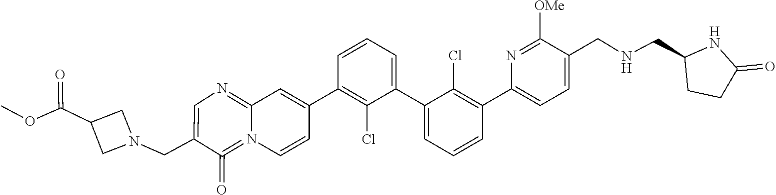

This compound was prepared in a similar fashion to Example 94, using azetidine-3-carboxylate HCl and (S)-tert-butyl ((6-(2,2′-dichloro-3′-(3-formyl-4-oxo-4H-pyrido[1,2-a]pyrimidin-8-yl)-[1,1′-biphenyl]-3-yl)-2-methoxypyridin-3-yl)methyl)((5-oxopyrrolidin-2-yl)methyl)carbamate. MS: m/z found 727 [M+H]⁺ (Method AF). ¹H NMR (400 MHz, DMSO-d₆): δ 9.02 (d, J=7.6 Hz, 1H), 8.31 (s, 1H), 7.83 (d, J=7.6 Hz, 1H), 7.77 (d, J=1.2 Hz, 1H), 7.70-7.67 (m, 3H), 7.65-7.62 (m, 1H), 7.58-7.55 (m, 2H), 7.50-7.46 (m, 2H), 7.28 (d, J=7.2 Hz, 1H), 3.93 (s, 3H), 3.73 (s, 2H), 3.64 (m, 4H), 3.56 (s, 2H), 3.50-3.48 (m, 2H), 3.30-3.28 (m, 3H), 2.56 (d, J=6 Hz, 2H), 2.18-2.08 (m, 3H), 1.76-1.67 (m, 1H).

### Example 163: 8-(2,2′-Dichloro-3′-(6-methoxy-5-(((((S)-5-oxopyrrolidin-2-yl)methyl) amino)methyl)pyridin-2-yl)-[1,1′-biphenyl]-3-yl)-3-((((3R,4S)-3-hydroxytetrahydro-2H-pyran-4-yl)amino)methyl)-4H-pyrido[1,2-a]pyrimidin-4-one)

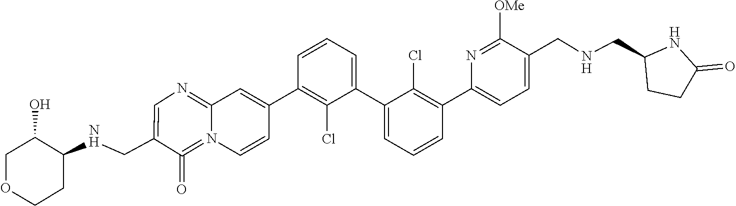

This compound was prepared in a similar fashion to Example 94, using (3R,4S)-4-aminotetrahydropyran-3-ol and (S)-tert-butyl ((6-(2,2′-dichloro-3′-(3-formyl-4-oxo-4H-pyrido[1,2-a]pyrimidin-8-yl)-[1,1′-biphenyl]-3-yl)-2-methoxypyridin-3-yl)methyl)((5-oxopyrrolidin-2-yl)methyl)carbamate. MS: m/z found 729 [M+H]⁺ (Method A). ¹H NMR (400 MHz, Methanol-d₄) δ 9.14 (d, J=7.6 Hz, 1H), 8.43 (s, 1H), 7.78 (d, J=1.2 Hz, 1H), 7.75 (d, J=7.6 Hz, 1H), 7.68-7.61 (m, 3H), 7.55-7.52 (m, 3H), 7.42-7.40 (m, 1H), 7.26 (d, J=7.6 Hz, 1H), 4.04-3.84 (m, 1OH), 3.44-3.37 (m, 2H), 3.06 (t, J=6.4 Hz, 1H), 2.73-2.69 (m, 2H), 2.58-2.57 (m, 1H), 2.38-2.28 (m, 3H), 2.16-2.13 (m, 1H), 1.86-1.81 (m, 1H), 1.52-1.43 (m, 1H).

### Example 166: (2S,4R)-1-((8-(2,2′-Dichloro-3′-(6-methoxy-5-(((((S)-5-oxopyrrolidin-2-yl)methyl)amino)methyl)pyridin-2-yl)-[1,1′-biphenyl]-3-yl)-4-oxo-4H-pyrido[1,2-a]pyrimidin-3-yl)methyl)-4-hydroxypyrrolidine-2-carboxylic acid

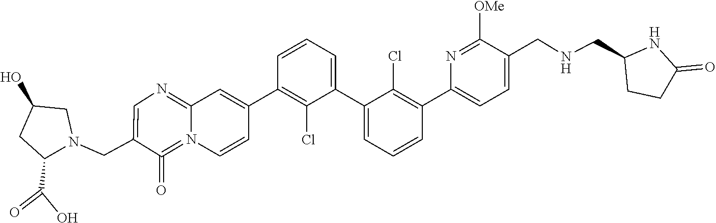

This compound was prepared in a similar fashion to Example 94, using (2S,4R)-4-hydroxypyrrolidine-2-carboxylic acid and (S)-tert-butyl ((6-(2,2′-dichloro-3′-(3-formyl-4-oxo-4H-pyrido[1,2-a]pyrimidin-8-yl)-[1,1′-biphenyl]-3-yl)-2-methoxypyridin-3-yl)methyl)((5-oxopyrrolidin-2-yl)methyl)carbamate. MS: m/z found 743 [M+H]+, (Method A). ¹H NMR (400 MHz, Methanol-d₄): δ 9.16 (d, J=6.8 Hz, 1H), 8.54 (s, 1H), 7.82 (d, J=1.2 Hz, 1H), 7.77 (d, J=7.6 Hz, 1H), 7.64-7.59 (m, 4H), 7.52-7.48 (m, 2H), 7.40 (m, 1H), 7.27 (d, J=7.2 Hz, 1H), 4.60-4.51 (m, 3H), 4.36-4.32 (m, 1H), 4.03 (s, 3H), 3.99-3.97 (m, 2H), 3.89-3.88 (m, 1H), 3.79-3.74 (m, 1H), 3.40-3.35 (m, 1H), 2.88-2.85 (m, 2H), 2.51-2.46 (m, 1H), 2.37-2.32 (m, 3H), 2.29-2.16 (m, 1H), 1.84-1.83 (m, 1H).

### Example 169: 8-(2,2′-Dichloro-3′-(6-methoxy-5-(((((S)-5-oxopyrrolidin-2-yl)methyl)amino)methyl)pyridin-2-yl)-[1,1′-biphenyl]-3-yl)-3-(((R)-3-hydroxypyrrolidin-1-yl)methyl)-4H-pyrido[1,2-a]pyrimidin-4-one

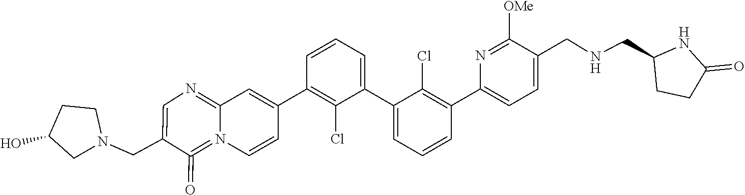

This compound was prepared in a similar fashion to Example 94, using (R)-pyrrolidin-3-ol and (S)-tert-butyl ((6-(2,2′-dichloro-3′-(3-formyl-4-oxo-4H-pyrido[1,2-a]pyrimidin-8-yl)-[1,1′-biphenyl]-3-yl)-2-methoxypyridin-3-yl)methyl)((5-oxopyrrolidin-2-yl)methyl)carbamate. MS: m/z found 699 [M+H]⁺, (Method C). ¹H NMR (400 MHz, Methanol-d₄): δ 9.10 (d, J=7.6 Hz, 1H), 8.44 (s, 1H), 8.32 (s, 1H), 7.77 (s, 1H), 7.69 (d, J=8 Hz, 1H), 7.55-7.51 (m, 4H), 7.44-7.42 (m, 2H), 7.32 (d, J=1.6 Hz, 1H), 7.18 (d, J=7.6 Hz, 1H), 4.47-4.45 (m, 1H), 4.31 (s, 2H), 3.94 (s, 3H), 3.90 (s, 2H), 3.85 (m, 1H), 3.47-3.37 (m, 1H), 3.34-3.33 (m, 2H), 3.27-3.26 (m, 1H), 2.79-2.76 (m, 2H), 2.28-2.21 (m, 3H), 1.96-1.95 (m, 1H), 1.76-1.75 (m, 1H).

### Example 182: (S)-3-(((8-(2,2′-Dichloro-3′-(6-methoxy-5-((((5-oxopyrrolidin-2-yl)methyl) amino)methyl)pyridin-2-yl)-[1,1′-biphenyl]-3-yl)-4-oxo-4H-pyrido[1,2-a]pyrimidin-3-yl)methyl)amino)propanoic acid

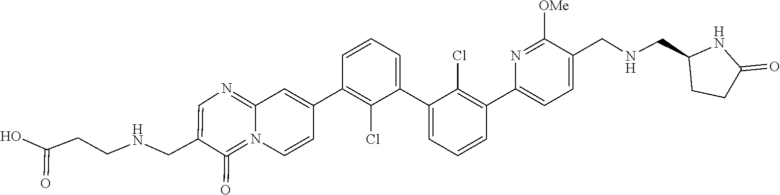

This compound was prepared in a similar fashion to Example 94, using 3-aminopropanoic acid and (S)-tert-butyl ((6-(2,2′-dichloro-3′-(3-formyl-4-oxo-4H-pyrido[1,2-a]pyrimidin-8-yl)-[1,1′-biphenyl]-3-yl)-2-methoxypyridin-3-yl)methyl)((5-oxopyrrolidin-2-yl)methyl)carbamate. MS: m/z found 701 [M+H]⁺ (Method AF). ¹H NMR (400 MHz, Methanol-d₄) δ 9.17-9.13 (m, 1H), 8.53 (s, 1H), 7.83-7.79 (m, 2H), 7.68-7.51 (m, 6H), 7.41-7.40 (m, 1H), 7.28 (dd, J=7.6 Hz, J=2 Hz, 1H), 4.30 (s, 2H), 4.05 (d, J=1.6 Hz, 3H), 4.00 (d, J=3.2 Hz, 2H), 3.96-3.93 (m, 1H), 3.28 (t, J=6 Hz, 2H), 2.93-2.83 (m, 2H), 2.58 (t, J=6 Hz, 2H), 2.39-2.28 (m, 3H), 1.93-1.82 (m, 1H).

### Example 184: (S)-2-(((8-(2,2′-Dichloro-3′-(6-methoxy-5-((((5-oxopyrrolidin-2-yl)methyl)amino)methyl)pyridin-2-yl)-[1,1′-biphenyl]-3-yl)-4-oxo-4H-pyrido[1,2-a]pyrimidin-3-yl)methyl)amino)-2-methylpropanoic acid

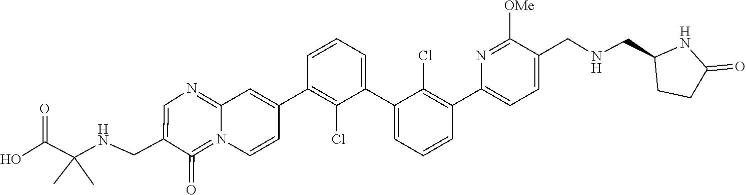

This compound was prepared in a similar fashion to Example 94, using 2-amino-2-methylpropanoic acid and (S)-tert-butyl ((6-(2,2′-dichloro-3′-(3-formyl-4-oxo-4H-pyrido[1,2-a]pyrimidin-8-yl)-[1,1′-biphenyl]-3-yl)-2-methoxypyridin-3-yl)methyl)((5-oxopyrrolidin-2-yl)methyl)carbamate. MS: m/z found 715 [M+H]⁺, (Method A). ¹H NMR (400 MHz, Methanol-d₄) δ 9.18 (d, J=7.6 Hz, 1H), 8.57 (s, 1H), 7.85 (s, 1H), 7.77 (d, J=7.2 Hz, 1H), 7.69-7.60 (m, 4H), 7.54-7.50 (m, 2H), 7.42-7.40 (m, 1H), 7.27 (d, J=7.2 Hz, 1H), 4.27 (s, 2H), 4.04 (s, 3H), 3.94-3.87 (m, 3H), 2.83-2.73 (m, 2H), 2.38-2.26 (m, 3H), 1.89-1.80 (m, 1H), 1.62 (s, 6H).

### Example 187: (S)—N-((8-(2,2′-Dichloro-3′-(6-methoxy-5-((((5-oxopyrrolidin-2-yl)methyl)amino)methyl)pyridin-2-yl)-[1,1′-biphenyl]-3-yl)-4-oxo-4H-pyrido[1,2-a]pyrimidin-3-yl)methyl)-N-methylglycine

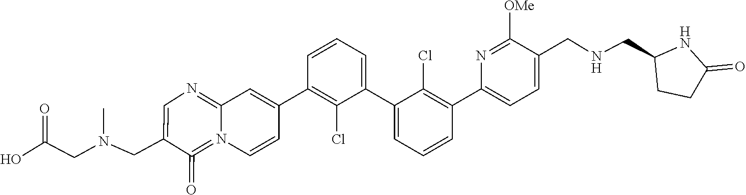

This compound was prepared in a similar fashion to Example 94, using 2-(methylamino)acetic acid and (S)-tert-butyl ((6-(2,2′-dichloro-3′-(3-formyl-4-oxo-4H-pyrido[1,2-a]pyrimidin-8-yl)-[1,1′-biphenyl]-3-yl)-2-methoxypyridin-3-yl)methyl)((5-oxopyrrolidin-2-yl)methyl)carbamate. MS: m/z found 701 [M+H]+, (Method A). ¹H NMR (400 MHz, DMSO-d₆): δ 9.04 (d, J=7.2 Hz, 1H), 8.42 (s, 1H), 7.90 (d, J=7.6 Hz, 1H), 7.79 (d, J=0.8 Hz, 1H), 7.68-7.55 (m, 6H), 7.51-7.50 (m, 2H), 7.35 (d, J=7.6 Hz, 1H), 4.03 (s, 2H), 3.95 (s, 3H), 3.85 (s, 2H), 3.82-3.78 (m, 1H), 3.34 (s, 3H), 2.91-2.87 (m, 2H), 2.46-2.43 (m, 3H), 2.18-2.11 (m, 3H), 1.76-1.73 (m, 1H).

### Example 189: (S)-((8-(2,2′-Dichloro-3′-(6-methoxy-5-((((5-oxopyrrolidin-2-yl)methyl)amino)methyl)pyridin-2-yl)-[1,1′-biphenyl]-3-yl)-4-oxo-4H-pyrido[1,2-a]pyrimidin-3-yl)methyl)glycine

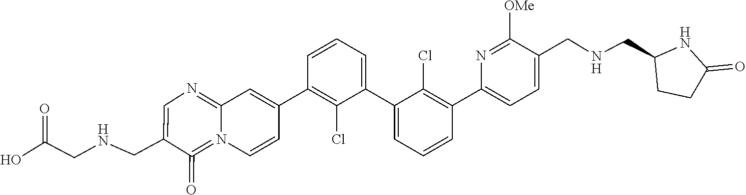

This compound was prepared in a similar fashion to Example 94, using 2-aminoacetic acid and (S)-tert-butyl ((6-(2,2′-dichloro-3′-(3-formyl-4-oxo-4H-pyrido[1,2-a]pyrimidin-8-yl)-[1,1′-biphenyl]-3-yl)-2-methoxypyridin-3-yl)methyl)((5-oxopyrrolidin-2-yl)methyl)carbamate. MS: m/z found 687 [M+H]⁺, (Method A). ¹H NMR (400 MHz, Methanol-d₄): δ 9.20 (d, J=7.6 Hz, 1H), 8.53 (s, 1H), 7.87-7.85 (m, 2H), 7.70-7.62 (m, 4H), 7.57-7.54 (m, 2H), 7.46 (d, J=1.2 Hz, 1H), 7.35 (d, J=7.6 Hz, 1H), 4.66 (s, 1H), 4.33 (s, 2H), 4.17 (d, J=2.0 Hz, 1H), 4.09 (s, 3H), 4.02-3.99 (m, 1H), 3.62 (s, 2H), 3.09-3.06 (m, 2H), 2.43-2.36 (m, 3H), 1.91-1.90 (m, 1H).

### Example 190: N-((8-(2,2′-Dichloro-3′-(6-methoxy-5-(((((S)-5-oxopyrrolidin-2-yl)methyl) amino)methyl)pyridin-2-yl)-[1,1′-biphenyl]-3-yl)-4-oxo-4H-pyrido[1,2-a]pyrimidin-3-yl)methyl)-N-methyl-L-alanine

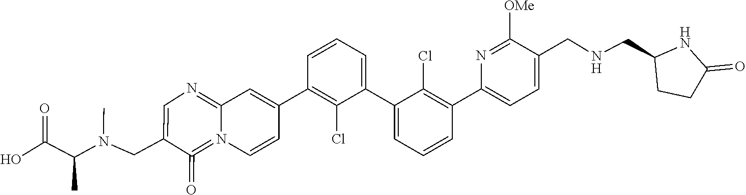

This compound was prepared in a similar fashion to Example 94, using (2S)-2-(methylamino)propanoic acid and (S)-tert-butyl ((6-(2,2′-dichloro-3′-(3-formyl-4-oxo-4H-pyrido[1,2-a]pyrimidin-8-yl)-[1,1′-biphenyl]-3-yl)-2-methoxypyridin-3-yl)methyl)((5-oxopyrrolidin-2-yl)methyl)carbamate. MS: m/z found 715 [M+H]+, (Method A). ¹H NMR (400 MHz, Methanol-d₄): δ 9.21 (d, J=7.2 Hz, 1H), 8.61 (s, 1H), 7.89-7.84 (m, 2H), 7.70-7.63 (m, 4H), 7.55-7.53 (m, 2H), 7.45 (d, J=1.6 Hz, 1H), 7.34 (d, J=7.2 Hz, 1H), 4.49-4.40 (m, 2H), 4.13-4.08 (m, 5H), 4.00-3.86 (m, 2H), 3.04-3.01 (m, 2H), 2.88 (s, 3H), 2.42-2.34 (m, 3H), 1.90-1.88 (m, 1H), 1.62 (d, J=7.2 Hz, 3H).

### Example 192: (S)-Methyl 1-((8-(2,2′-dichloro-3′-(6-methoxy-5-(((((S)-5-oxopyrrolidin-2-yl)methyl)amino)methyl)pyridin-2-yl)-[1,1′-biphenyl]-3-yl)-4-oxo-4H-pyrido[1,2-a]pyrimidin-3-yl)methyl)pyrrolidine-3-carboxylate

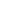

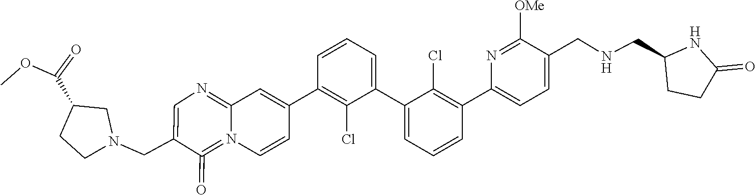

### (S)-Methyl pyrrolidine-3-carboxylate

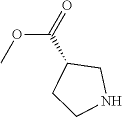

To a mixture of (S)-pyrrolidine-3-carboxylic acid (0.3 g, 2.6 mmol) in MeOH (5 mL), was added sulfurous dichloride (0.38 mL, 5.2 mmol) dropwise at rt. The reaction was stirred at rt for 1 hr under N₂, then was concentrated to afford (S)-methyl pyrrolidine-3-carboxylate as the hydrochloride acid salt as a light yellow solid without purification (0.46 g, crude). ¹H NMR (400 MHz, Methanol-d₄): δ3.74 (s, 3H), 3.58-3.47 (m, 2H), 3.36-3.30 (m, 3H), 2.37-2.22 (m, 2H).

### (S)-Methyl 1-((8-(2,2′-dichloro-3′-(6-methoxy-5-(((((S)-5-oxopyrrolidin-2-yl)methyl) amino)methyl)pyridin-2-yl)-[1,1′-biphenyl]-3-yl)-4-oxo-4H-pyrido[1,2-a]pyrimidin-3-yl)methyl)pyrrolidine-3-carboxylate

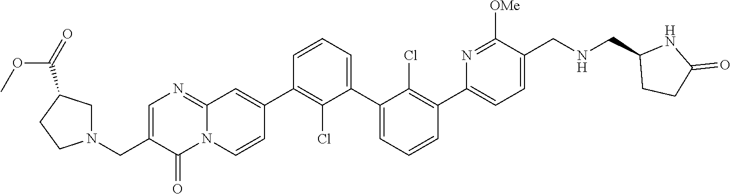 [SMILES: COC(=O)[C@H]1CCN(Cc2cnc3cc(-c4cccc(-c5cccc(-c6ccc(CNC[C@@H]7CCC(=O)N7)c(OC)n6)c5Cl)c4Cl)ccn3c2=O)C1]

This compound was prepared in a similar fashion to Example 94, using (S)-methyl pyrrolidine-3-carboxylate and (S)-tert-butyl ((6-(2,2′-dichloro-3′-(3-formyl-4-oxo-4H-pyrido[1,2-a]pyrimidin-8-yl)-[1,1′-biphenyl]-3-yl)-2-methoxypyridin-3-yl)methyl)((5-oxopyrrolidin-2-yl)methyl)carbamate. MS: m/z found 713 [M+H]⁺, (Method A). ¹H NMR (400 MHz, Methanol-d₄): δ9.19 (d, J=7.6 Hz, 1H), 8.53 (s, 1H), 7.86-7.84 (m, 2H), 7.68-7.62 (m, 4H), 7.54-7.51 (m, 2H), 7.42 (dd, J=7.6 Hz, J=1.6 Hz, 1H), 7.33 (d, J=7.2 Hz, 1H), 4.38 (s, 2H), 4.19 (m, 2H), 4.07 (s, 3H), 4.03-4.00 (m, 1H), 3.74 (s, 3H), 3.63-3.60 (m, 2H), 3.45-3.39 (m, 3H), 3.13-3.08 (m, 2H), 2.42-2.30 (m, 5H), 1.91-1.67 (m, 1H).

### Example 200: (S)-2-(((8-(2,2′-Dichloro-3′-(6-methoxy-5-((((5-oxopyrrolidin-2-yl)methyl)amino)methyl)pyridin-2-yl)-[1,1′-biphenyl]-3-yl)-4-oxo-4H-pyrido[1,2-a]pyrimidin-3-yl)methyl)(methyl)amino)-N-methylacetamide

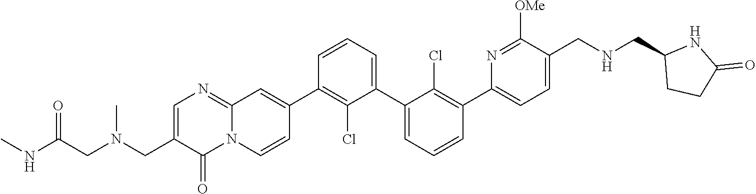 [SMILES: CNC(=O)CN(C)Cc1cnc2cc(-c3cccc(-c4cccc(-c5ccc(CNC[C@@H]6CCC(=O)N6)c(OC)n5)c4Cl)c3Cl)ccn2c1=O]

### N-Methyl-2-(methylamino)acetamide

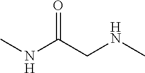

Ethyl 2-chloroacetate (5.30 mL, 49.8 mmol) was added to an excess of 40% concentrated aqueous methylamine (70 mL). The reaction was stirred for 12 hr at r.t. The resulting solution was saturated with sodium chloride and extracted with dichloromethane (80 mL), dried over sodium sulfate, and filtered through a layer of magnesium sulfate. The filtrate was concentrated in vacuum to afford the crude product N-methyl-2-(methylamino)acetamide as a colorless oil (1.76 g, crude) which was used into the next step without further purification. ¹H NMR (400 MHz, Methanol-d₄): δ 3.19 (s, 2H), 2.78 (s, 3H), 2.37 (s, 3H).

### (S)-2-(((8-(2,2′-Dichloro-3′-(6-methoxy-5-((((5-oxopyrrolidin-2-yl)methyl)amino) methyl)pyridin-2-yl)-[1,1′-biphenyl]-3-yl)-4-oxo-4H-pyrido[1,2-a]pyrimidin-3-yl) methyl)(methyl)amino)-N-methylacetamide

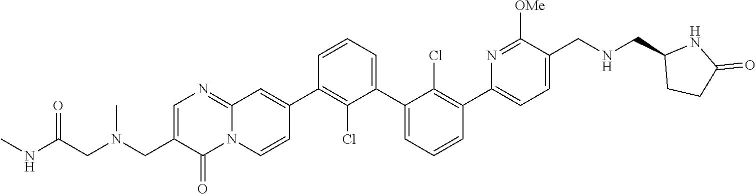 [SMILES: CNC(=O)CN(C)Cc1cnc2cc(-c3cccc(-c4cccc(-c5ccc(CNC[C@@H]6CCC(=O)N6)c(OC)n5)c4Cl)c3Cl)ccn2c1=O]

This compound was prepared in a similar fashion to Example 94, using N-methyl-2-(methylamino)acetamide and (S)-tert-butyl ((6-(2,2′-dichloro-3′-(3-formyl-4-oxo-4H-pyrido[1,2-a]pyrimidin-8-yl)-[1,1′-biphenyl]-3-yl)-2-methoxypyridin-3-yl)methyl)((5-oxopyrrolidin-2-yl)methyl)carbamate. MS: m/z found 714 [M+H]+, (Method A). ¹H NMR (400 MHz, Methanol-d₄): δ 9.21 (d, J=7.2 Hz, 1H), 8.61 (s, 1H), 7.89-7.84 (m, 2H), 7.70-7.63 (m, 4H), 7.55-7.53 (m, 2H), 7.45 (d, J=1.6 Hz, 1H), 7.34 (d, J=7.2 Hz, 1H), 4.49-4.40 (m, 2H), 4.13-4.08 (m, 5H), 4.00-3.86 (m, 2H), 3.04-3.01 (m, 2H), 2.88 (s, 3H), 2.42-2.34 (m, 3H), 1.90-1.88 (m, 1H), 1.62 (d, J=7.2 Hz, 3H).

### Example 202: (S)-2-(((8-(2,2′-Dichloro-3′-(6-methoxy-5-((((5-oxopyrrolidin-2-yl)methyl)amino)methyl)pyridin-2-yl)-[1,1′-biphenyl]-3-yl)-4-oxo-4H-pyrido[1,2-a]pyrimidin-3-yl)methyl)amino)-N-methylacetamide

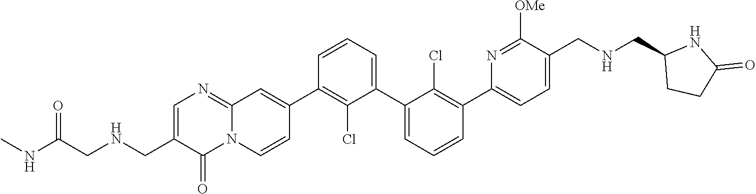 [SMILES: CNC(=O)CNCc1cnc2cc(-c3cccc(-c4cccc(-c5ccc(CNC[C@@H]6CCC(=O)N6)c(OC)n5)c4Cl)c3Cl)ccn2c1=O]

This compound was prepared in a similar fashion to Example 94, using 2-amino-N-methylacetamide and (S)-tert-butyl ((6-(2,2′-dichloro-3′-(3-formyl-4-oxo-4H-pyrido[1,2-a]pyrimidin-8-yl)-[1,1′-biphenyl]-3-yl)-2-methoxypyridin-3-yl)methyl)((5-oxopyrrolidin-2-yl)methyl)carbamate. MS: m/z found 700.1 [M+H]⁺, (Method A). ¹H NMR (400 MHz, Methanol-d₄): δ 9.18 (d, J=7.2 Hz, 1H), 8.45 (s, 1H), 8.42 (br, 1H), 7.84-7.82 (m, 2H), 7.70-7.53 (m, 6H), 7.43 (d, J=7.2 Hz, 1H), 7.32 (d, J=7.2 Hz, 1H), 4.12-3.96 (m, 8H), 3.56 (s, 2H), 3.01-2.92 (m, 2H), 2.81 (s, 3H), 2.44-2.30 (m, 3H), 1.93-1.84 (m, 1H).

### Example 209: (S)-2-(((8-(2,2′-Dichloro-3′-(6-methoxy-5-((((5-oxopyrrolidin-2-yl)methyl)amino)methyl)pyridin-2-yl)-[1,1′-biphenyl]-3-yl)-4-oxo-4H-pyrido[1,2-a]pyrimidin-3-yl)methyl)amino)-N,2-dimethylpropanamide

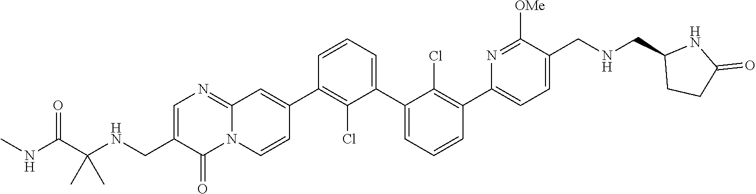 [SMILES: CNC(=O)C(C)(C)NCc1cnc2cc(-c3cccc(-c4cccc(-c5ccc(CNC[C@@H]6CCC(=O)N6)c(OC)n5)c4Cl)c3Cl)ccn2c1=O]

This compound was prepared in a similar fashion to Example 94, using 2-amino-N,2-dimethylpropanamide and (S)-tert-butyl ((6-(2,2′-dichloro-3′-(3-formyl-4-oxo-4H-pyrido[1,2-a]pyrimidin-8-yl)-[1,1′-biphenyl]-3-yl)-2-methoxypyridin-3-yl)methyl)((5-oxopyrrolidin-2-yl)methyl)carbamate. MS: m/z found 728 [M+H]+, (Method A). ¹H NMR (400 MHz, Methanol-d₄): δ 9.19 (d, J=8.0 Hz, 1H), 8.44 (s, 1H), 7.81-7.80 (m, 2H), 7.68-7.52 (m, 6H), 7.44-7.42 (dd, J=7.6 Hz, J=1.6 Hz, 1H), 7.31 (d, J=7.2 Hz, 1H), 4.07 (s, 3H), 4.01 (d, J=3.2 Hz, 2H), 3.94-3.91 (m, 1H), 3.85 (s, 2H), 2.94-2.86 (m, 2H), 2.85 (s, 3H), 2.40-2.33 (m, 3H), 1.88-1.87 (m, 1H), 1.48 (s, 6H).

### Example 210: (S)-3-(((8-(2,2′-dichloro-3′-(6-methoxy-5-((((5-oxopyrrolidin-2-yl)methyl)amino)methyl)pyridin-2-yl)-[1,1′-biphenyl]-3-yl)-4-oxo-4H-pyrido[1,2-a]pyrimidin-3-yl)methyl)amino)-N-methylpropanamide

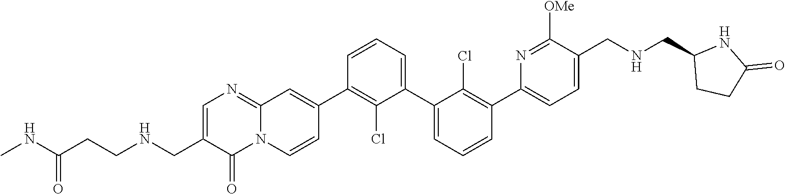 [SMILES: CNC(=O)CCNCc1cnc2cc(-c3cccc(-c4cccc(-c5ccc(CNC[C@@H]6CCC(=O)N6)c(OC)n5)c4Cl)c3Cl)ccn2c1=O]

This compound was prepared in a similar fashion to Example 94, using 3-amino-N-methylpropanamide and (S)-tert-butyl ((6-(2,2′-dichloro-3′-(3-formyl-4-oxo-4H-pyrido[1,2-a]pyrimidin-8-yl)-[1,1′-biphenyl]-3-yl)-2-methoxypyridin-3-yl)methyl)((5-oxopyrrolidin-2-yl)methyl)carbamate. MS: m/z found 714 [M+H]+, (Method A). ¹H NMR (400 MHz, methanol-d₄): δ 9.18 (d, J=7.2 Hz, 1H), 8.51 (s, 1H), 7.88-7.86 (m, 2H), 7.68-7.61 (m, 4H), 7.53-7.51 (m, 2H), 7.43 (d, J=8 Hz, 1H), 7.35 (d, J=7.2 Hz, 1H), 4.31 (s, 2H), 4.26-4.25 (m, 2H), 4.03 (s, 3H), 4.02-4.01 (m, 1H), 3.38-3.36 (m, 2H), 3.17-3.14 (m, 2H), 2.74 (s, 3H), 2.69-2.66 (m, 2H), 2.39-2.36 (m, 3H), 1.92-1.87 (m, 1H).

### Example 255: (S)-1-((8-(2,2′-Dichloro-3′-(6-methoxy-5-((((5-oxopyrrolidin-2-yl)methyl)amino)methyl)pyridin-2-yl)-[1,1′-biphenyl]-3-yl)-4-oxo-4H-pyrido[1,2-a]pyrimidin-3-yl)methyl)-N-methylazetidine-3-carboxamide

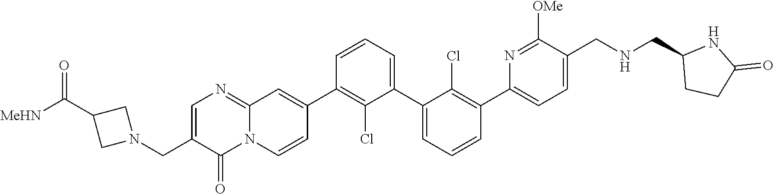 [SMILES: CNC(=O)C1CN(Cc2cnc3cc(-c4cccc(-c5cccc(-c6ccc(CNC[C@@H]7CCC(=O)N7)c(OC)n6)c5Cl)c4Cl)ccn3c2=O)C1]

### (S)-1-((8-(3′-(5-(((tert-Butoxycarbonyl)((5-oxopyrrolidin-2-yl)methyl)amino)methyl)-6-methoxypyridin-2-yl)-2,2′-dichloro-[1,1′-biphenyl]-3-yl)-4-oxo-4H-pyrido[1,2-a]pyrimidin-3-yl)methyl)azetidine-3-carboxylic acid

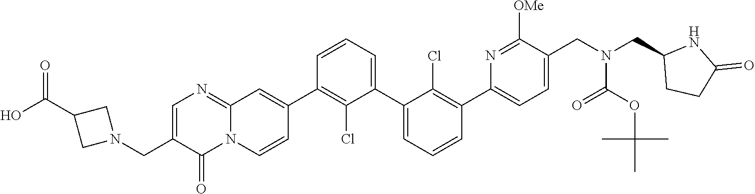 [SMILES: COc1nc(-c2cccc(-c3cccc(-c4ccn5c(=O)c(CN6CC(C(=O)O)C6)cnc5c4)c3Cl)c2Cl)ccc1CN(C[C@@H]1CCC(=O)N1)C(=O)OC(C)(C)C]

This compound was prepared in a similar fashion to Example 94, using azetidine-3-carboxylic acid and (S)-tert-butyl ((6-(2,2′-dichloro-3′-(3-formyl-4-oxo-4H-pyrido[1,2-a]pyrimidin-8-yl)-[1,1′-biphenyl]-3-yl)-2-methoxypyridin-3-yl)methyl)((5-oxopyrrolidin-2-yl)methyl)carbamate. MS: m/z found 813 [M+H]⁺ (Method J).

### tert-Butyl (S)-((6-(2,2′-Dichloro-3′-(3-((3-(methylcarbamoyl)azetidin-1-yl)methyl)-4-oxo-4H-pyrido[1,2-a]pyrimidin-8-yl)-[1,1′-biphenyl]-3-yl)-2-methoxypyridin-3-yl)methyl)((5-oxopyrrolidin-2-yl)methyl)carbamate

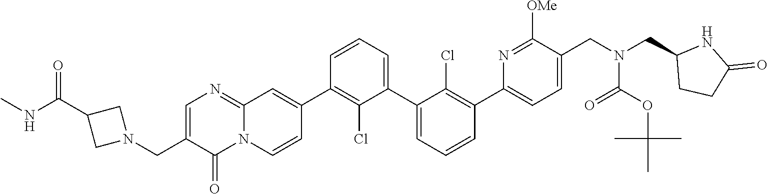 [SMILES: CNC(=O)C1CN(Cc2cnc3cc(-c4cccc(-c5cccc(-c6ccc(CN(C[C@@H]7CCC(=O)N7)C(=O)OC(C)(C)C)c(OC)n6)c5Cl)c4Cl)ccn3c2=O)C1]

To a mixture (S)-1-((8-(3′-(5-(((tert-butoxycarbonyl)((5-oxopyrrolidin-2-yl)methyl) amino)methyl)-6-methoxypyridin-2-yl)-2,2′-dichloro-[1,1′-biphenyl]-3-yl)-4-oxo-4H-pyrido[1,2-a]pyrimidin-3-yl)methyl)azetidine-3-carboxylic acid (0.56 g, 0.69 mmol) in DMF (8 mL) was added methylamine HCl (0.14 g, 2.06 mmol), 1-(3-Dimethylaminopropyl)-3-ethylcarbodiimide hydrochloride (0.66 g, 3.44 mmol) and 1-hydroxybenzotriazole (0.23 g, 1.72 mmol). The mixture was stirred at r.t. for 1 hr, to yield (S)-tert-butyl ((6-(2,2′-dichloro-3′-(3-((3-(methylcarbamoyl)azetidin-1-yl)methyl)-4-oxo-4H-pyrido[1,2-a]pyrimidin-8-yl)-[1,1′-biphenyl]-3-yl)-2-methoxypyridin-3-yl)methyl)((5-oxopyrrolidin-2-yl)methyl)carbamate as a yellow solution, which was used crude as such (m/z: 826 [M+H]⁺ observed (LCMS: Method J).

### (S)-1-((8-(2,2′-Dichloro-3′-(6-methoxy-5-((((5-oxopyrrolidin-2-yl)methyl)amino) methyl) pyridin-2-yl)-[1,1′-biphenyl]-3-yl)-4-oxo-4H-pyrido[1,2-a]pyrimidin-3-yl)methyl)-N-methylazetidine-3-carboxamide

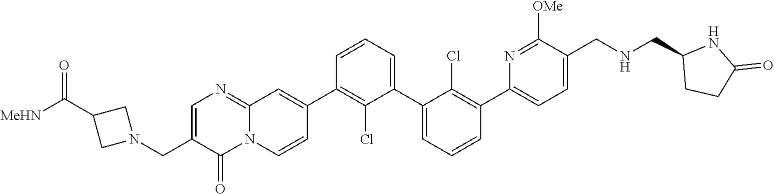 [SMILES: CNC(=O)C1CN(Cc2cnc3cc(-c4cccc(-c5cccc(-c6ccc(CNC[C@@H]7CCC(=O)N7)c(OC)n6)c5Cl)c4Cl)ccn3c2=O)C1]

A solution of (S)-tert-butyl ((6-(2,2′-dichloro-3′-(3-((3-(methylcarbamoyl)azetidin-1-yl)methyl)-4-oxo-4H-pyrido[1,2-a]pyrimidin-8-yl)-[1,1′-biphenyl]-3-yl)-2-methoxypyridin-3-yl)methyl)((5-oxopyrrolidin-2-yl)methyl)carbamate in DMF (8 mL) was added trifluoroacetic acid (20 mL). The mixture was stirred at r.t. for 10 min, then filtered and concentrated under reduced pressure. The residue was purified by reverse phase HPLC to afford (S)-1-((8-(2,2′-dichloro-3′-(6-methoxy-5-((((5-oxopyrrolidin-2-yl)methyl)amino)methyl)pyridin-2-yl)-[1,1′-biphenyl]-3-yl)-4-oxo-4H-pyrido[1,2-a]pyrimidin-3-yl)methyl)-N-methylazetidine-3-carboxamide as light yellow solid (77 mg, 15% yield, MS: 726.2 [M+H]⁺ MS method A). ¹H NMR (400 MHz, Methanol-d₄): δ 9.13 (d, J=7.2 Hz, 1H), 8.38 (s, 1H), 7.78-7.74 (m, 2H), 7.67-7.61 (m, 3H), 7.56-7.53 (m, 3H), 7.42 (dd, J=1.6 Hz, J=8 Hz, 1H), 7.26 (d, J=7.6 Hz, 1H), 4.04 (s, 3H), 3.89-3.81 (m, 3H), 3.75 (s, 2H), 3.62 (t, J=7.6 Hz, 2H), 3.51 (t, J=7.6 Hz, 2H), 3.31-3.25 (m, 1H), 2.76-2.67 (m, 5H), 2.38-2.25 (m, 3H), 1.88-1.79 (m, 1H).

### Example 257: (S)-1-((8-(2,2′-Dichloro-3′-(6-methoxy-5-(((((S)-5-oxopyrrolidin-2-yl)methyl)amino)methyl)pyridin-2-yl)-[1,1′-biphenyl]-3-yl)-4-oxo-4H-pyrido[1,2-a]pyrimidin-3-yl)methyl)-N-methylpyrrolidine-3-carboxamide

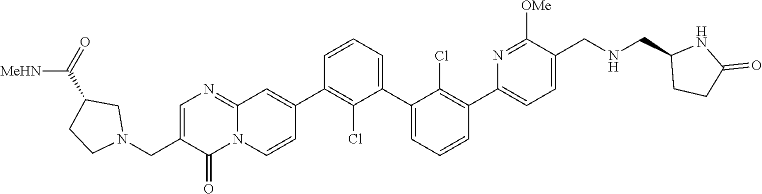 [SMILES: CNC(=O)[C@H]1CCN(Cc2cnc3cc(-c4cccc(-c5cccc(-c6ccc(CNC[C@@H]7CCC(=O)N7)c(OC)n6)c5Cl)c4Cl)ccn3c2=O)C1]

This compound was prepared in a similar fashion to Example 94, using (S)-pyrrolidine-3-carboxylic acid e and (S)-tert-butyl ((6-(2,2′-dichloro-3′-(3-formyl-4-oxo-4H-pyrido[1,2-a]pyrimidin-8-yl)-[1,1′-biphenyl]-3-yl)-2-methoxypyridin-3-yl)methyl)((5-oxopyrrolidin-2-yl)methyl)carbamate. MS: m/z found 740 [M+H]⁺, (Method A). ¹H NMR (400 MHz, Methanol-d₄): δ 9.15 (d, J=7.6 Hz, 1H), 8.44 (s, 1H), 7.79-7.79 (m, 1H), 7.76-7.74 (m, 1H), 7.69-7.60 (m, 3H), 7.56-7.51 (m, 3H), 7.41 (dd, J=1.6 Hz, J=7.6 Hz, 1H), 7.26 (d, J=7.6 Hz, 1H), 4.04 (s, 3H), 3.89-3.85 (m, 3H), 3.82-3.81 (m, 2H), 3.03-2.96 (m, 3H), 2.78-2.68 (m, 7H), 2.38-2.27 (m, 3H), 2.11-2.01 (m, 2H), 1.85-1.79 (m, 1H).

### Example 263: (S)-2-(((8-(2,2′-dichloro-3′-(6-methoxy-5-(((((S)-5-oxopyrrolidin-2-yl)methyl)amino)methyl)pyridin-2-yl)-[1,1′-biphenyl]-3-yl)-4-oxo-4H-pyrido[1,2-a]pyrimidin-3-yl)methyl)(methyl)amino)-N-methylpropanamide

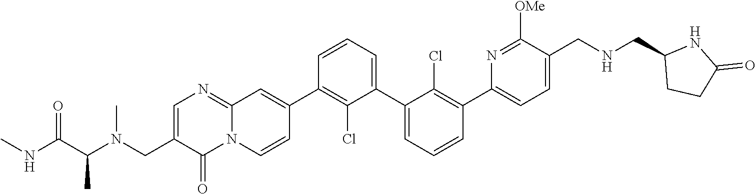 [SMILES: CNC(=O)[C@H](C)N(C)Cc1cnc2cc(-c3cccc(-c4cccc(-c5ccc(CNC[C@@H]6CCC(=O)N6)c(OC)n5)c4Cl)c3Cl)ccn2c1=O]

### N-(tert-butoxycarbonyl)-N-methyl-L-alanine

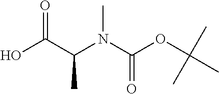

To a mixture of (S)-2-(methylamino)propanoic acid (1 g, 9.7 mmol) in MeOH (10 mL) was added triethylamine (2 mL, 15 mmol) and di-tert-butyl dicarbonate (2.7 mL, 12 mmol). Then the reaction was stirred at r.t. for 1 hr under N₂. The mixture was concentrated to afford N-(tert-butoxycarbonyl)-N-methyl-L-alanine (2.2 g, crude, m/z: 148 [(M-t-Butyl)+H]⁺ Method AD) was obtained as a colorless oil without purification.

### (S)-tert-Butyl methyl(1-(methylamino)-1-oxopropan-2-yl)carbamate

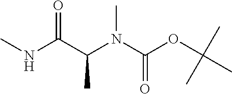

To a mixture of (S)-2-((tert-butoxycarbonyl)(methyl)amino)propanoic acid (1 g, 4.9 mmol) in DMF (15 mL) was added methylamine HCl (0.85 g, 12.3 mmol) and 1-hydroxybenzotriazole (1 g, 7.4 mmol). The mixture was cooled to 0° C., then 1-(3-Dimethylaminopropyl)-3-ethylcarbodiimide hydrochloride (2.8 g, 15 mmol) was added. The reaction was stirred at r.t. for 12 hr under N₂. Water was then added (50 mL). The mixture was extracted with EtOAc (50 mL×2). The combined organic phase was dried with anhydrous sodium sulfate, filtered and concentrated to afford (S)-tert-butyl methyl(1-(methylamino)-1-oxopropan-2-yl)carbamate (0.6 g, 56% yield) as a colorless oil without purification. ¹H NMR (400 MHz, CDCl₃): δ6.15-5.98 (m, 1H), 4.67-4.51 (m, 1H), 2.78 (d, J=4.8 Hz, 3H), 2.75 (s, 3H), 1.44 (s, 9H), 1.31 (d, J=7.2 Hz, 3H).

### (S)—N-Methyl-2-(methylamino)propanamide

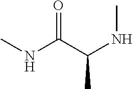

To a mixture of tert-butyl N-methyl-N-[(1S)-1-methyl-2-(methylamino)-2-oxo-ethyl]carbamate (0.6 g, 2.8 mmol) in DCM (5 mL) was added trifluoroacetic acid (1 mL, 13 mmol), then the mixture was stirred at r.t. for 2 hr under N₂. The mixture was concentrated to afford (S)—N-methyl-2-(methylamino)propanamide (0.7 g, crude) as a colorless oil. ¹H NMR (400 MHz, CDCl₃): δ4.22-4.21 (m, 1H), 2.87 (d, J=4.8 Hz, 3H), 2.78 (s, 3H), 1.54 (d, J=6.8 Hz, 3H).

### tert-Butyl ((6-(2,2′-Dichloro-3′-(3-((methyl((S)-1-(methylamino)-1-oxopropan-2-yl)amino)methyl)-4-oxo-4H-pyrido[1,2-a]pyrimidin-8-yl)-[1,1′-biphenyl]-3-yl)-2-methoxypyridin-3-yl)methyl)(((S)-5-oxopyrrolidin-2-yl)methyl)carbamate

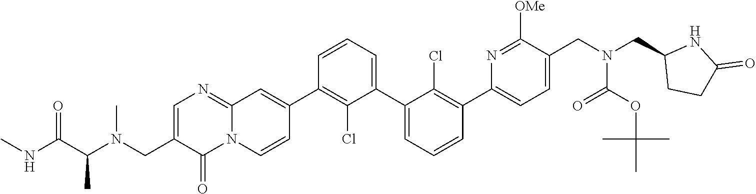 [SMILES: CNC(=O)[C@H](C)N(C)Cc1cnc2cc(-c3cccc(-c4cccc(-c5ccc(CN(C[C@@H]6CCC(=O)N6)C(=O)OC(C)(C)C)c(OC)n5)c4Cl)c3Cl)ccn2c1=O]

This compound was prepared in a similar fashion to Example 94, using (S)—N-methyl-2-(methylamino)propenamide and (S)-tert-butyl ((6-(2,2′-dichloro-3′-(3-formyl-4-oxo-4H-pyrido[1,2-a]pyrimidin-8-yl)-[1,1′-biphenyl]-3-yl)-2-methoxypyridin-3-yl)methyl)((5-oxopyrrolidin-2-yl)methyl)carbamate. MS: m/z found 828 [M+H]⁺, (Method AD).

### (S)-2-(((8-(2,2′-Dichloro-3′-(6-methoxy-5-(((((S)-5-oxopyrrolidin-2-yl)methyl)amino)methyl)pyridin-2-yl)-[1,1′-biphenyl]-3-yl)-4-oxo-4H-pyrido[1,2-a]pyrimidin-3-yl)methyl)(methyl)amino)-N-methylpropanamide

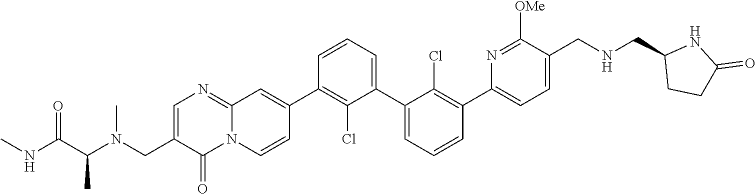 [SMILES: CNC(=O)[C@H](C)N(C)Cc1cnc2cc(-c3cccc(-c4cccc(-c5ccc(CNC[C@@H]6CCC(=O)N6)c(OC)n5)c4Cl)c3Cl)ccn2c1=O]

To the above solution of tert-butyl ((6-(2,2′-dichloro-3′-(3-((methyl((S)-1-(methylamino)-1-oxopropan-2-yl)amino)methyl)-4-oxo-4H-pyrido[1,2-a]pyrimidin-8-yl)-[1,1′-biphenyl]-3-yl)-2-methoxypyridin-3-yl)methyl)(((S)-5-oxopyrrolidin-2-yl)methyl)carbamate in DMF (3 mL) was added trifluoroacetic acid (4 mL, 54 mmol). The mixture was stirred at r.t. for 0.5 hr under N₂, then concentrated down. The residue was purified by reverse phase HPLC to afford (S)-2-(((8-(2,2′-dichloro-3′-(6-methoxy-5-(((((S)-5-oxopyrrolidin-2-yl)methyl)amino)methyl)pyridin-2-yl)-[1,1′-biphenyl]-3-yl)-4-oxo-4H-pyrido[1,2-a]pyrimidin-3-yl)methyl)(methyl)amino)-N-methylpropanamide (26.9 mg, 9% yield, m/z: 728 [M+H]*, observed, Method AD) as a white solid. ¹H NMR (400 MHz, Methanol-d₄): δ9.18 (d, J=7.6 Hz, 1H), 8.40 (s, 1H), 7.80 (d, J=1.2 Hz, 1H), 7.75 (d, J=7.2 Hz, 1H), 7.67-7.62 (m, 3H), 7.57-7.53 (m, 3H), 7.41 (dd, J=1.6 Hz, J=6 Hz, 1H), 7.26 (d, J=7.6 Hz, 1H), 4.04 (s, 3H), 3.89-3.85 (m, 3H), 3.61 (q, J=13.6 Hz, 2H), 2.85 (s, 3H), 2.75-2.68 (m, 3H), 2.38-2.29 (m, 6H), 1.89-1.81 (m, 1H), 1.33 (d, J=7.2 Hz, 3H).

### Example 118: (S)-1-((6-(2,2′-Dichloro-3′-(4-oxo-3-(((((S)-5-oxopyrrolidin-2-yl)methyl)amino)methyl)-4H-pyrido[1,2-a]pyrimidin-8-yl)-[1,1′-biphenyl]-3-yl)-2-methoxypyridin-3-yl)methyl)pyrrolidine-3-carboxylic acid

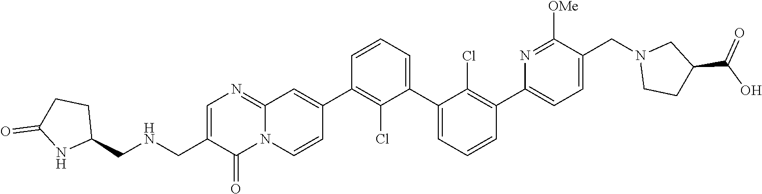 [SMILES: COc1nc(-c2cccc(-c3cccc(-c4ccn5c(=O)c(CNC[C@@H]6CCC(=O)N6)cnc5c4)c3Cl)c2Cl)ccc1CN1CC[C@H](C(=O)O)C1]

### (S)-tert-Butyl ((8-(2,2′-dichloro-3′-(5-formyl-6-methoxypyridin-2-yl)-[1,1′-biphenyl]-3-yl)-4-oxo-4H-pyrido[1,2-a]pyrimidin-3-yl)methyl)((5-oxopyrrolidin-2-yl)methyl)carbamate

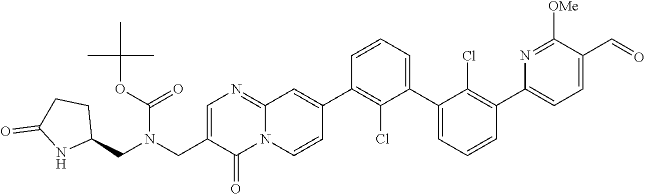 [SMILES: COc1nc(-c2cccc(-c3cccc(-c4ccn5c(=O)c(CN(C[C@@H]6CCC(=O)N6)C(=O)OC(C)(C)C)cnc5c4)c3Cl)c2Cl)ccc1C=O]

To a mixture of (S)-tert-butyl ((8-(3-bromo-2-chlorophenyl)-4-oxo-4H-pyrido[1,2-a]pyrimidin-3-yl)methyl)((5-oxopyrrolidin-2-yl)methyl)carbamate (0.5 g, 0.89 mmol and 6-(2-chloro-3-(4,4,5,5-tetramethyl-1,3-dioxolan-2-yl)phenyl)-2-methoxynicotinaldehyde (0.74 g, 0.89 mmol, 45% purity) in dioxane/H₂O mixture (5:1, 18 mL) was added potassium phosphate (0.38 g, 1.78 mmol) and [1,1′-Bis(di-tert-butylphosphino)ferrocene]dichloropalladium(II) (58 mg, 89 umol). Then the mixture was stirred at 110° C. for 1 h under N₂, then concentrated. The residue was purified by normal phase SiO₂ chromatography (0-15% EtOAc/petroleum ether) to afford (S)-tert-butyl ((8-(2,2′-dichloro-3′-(5-formyl-6-methoxypyridin-2-yl)-[1,1′-biphenyl]-3-yl)-4-oxo-4H-pyrido[1,2-a]pyrimidin-3-yl)methyl)((5-oxopyrrolidin-2-yl)methyl)carbamate as a brown solid (0.7 g, crude, m/z: 728 [M+H]⁺ observed, Method AB).

### (S)-1-((6-(3′-(3-(((tert-Butoxycarbonyl)(((S)-5-oxopyrrolidin-2-yl)methyl)amino)methyl)-4-oxo-4H-pyrido[1,2-a]pyrimidin-8-yl)-2,2′-dichloro-[1,1′-biphenyl]-3-yl)-2-methoxypyridin-3-yl)methyl)pyrrolidine-3-carboxylic acid

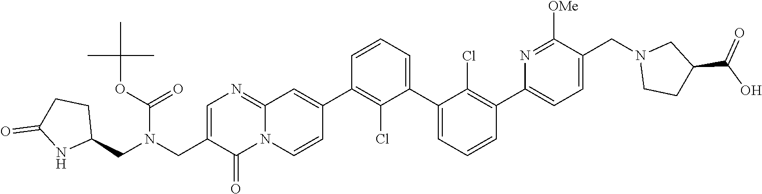 [SMILES: COc1nc(-c2cccc(-c3cccc(-c4ccn5c(=O)c(CN(C[C@@H]6CCC(=O)N6)C(=O)OC(C)(C)C)cnc5c4)c3Cl)c2Cl)ccc1CN1CC[C@H](C(=O)O)C1]

To a mixture of (S)-tert-butyl ((8-(2,2′-dichloro-3′-(5-formyl-6-methoxypyridin-2-yl)-[1,1′-biphenyl]-3-yl)-4-oxo-4H-pyrido[1,2-a]pyrimidin-3-yl)methyl)((5-oxopyrrolidin-2-yl)methyl)carbamate (0.35 g, 0.48 mmol) and (S)-pyrrolidine-3-carboxylic acid (0.16 g, 1.44 mmol) in DCM (5 mL) was added sodium acetate (0.2 g, 2.4 mmol). The mixture was stirred at r.t. for 0.5 h, then sodium triacetyloxyborohydride (0.31 g, 1.44 mmol) was added to the mixture and stirred at r.t. for 2.5 h under N₂. The mixture was filtered and concentrated to afford (S)-1-((6-(3′-(3-(((tert-butoxycarbonyl)(((S)-5-oxopyrrolidin-2-yl)methyl)amino)methyl)-4-oxo-4H-pyrido[1,2-a]pyrimidin-8-yl)-2,2′-dichloro-[1,1′-biphenyl]-3-yl)-2-methoxypyridin-3-yl)methyl)pyrrolidine-3-carboxylic acid as a crude brown oil (0.5 g, m/z: 827 [M+H]⁺ observed, Method AB).

### (S)-1-((6-(2,2′-Dichloro-3′-(4-oxo-3-(((((S)-5-oxopyrrolidin-2-yl)methyl)amino)methyl)-4H-pyrido[1,2-a]pyrimidin-8-yl)-[1,1′-biphenyl]-3-yl)-2-methoxypyridin-3-yl)methyl) pyrrolidine-3-carboxylic acid

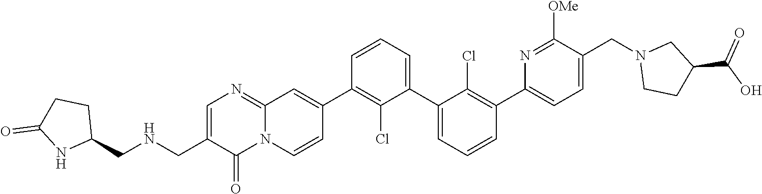 [SMILES: COc1nc(-c2cccc(-c3cccc(-c4ccn5c(=O)c(CNC[C@@H]6CCC(=O)N6)cnc5c4)c3Cl)c2Cl)ccc1CN1CC[C@H](C(=O)O)C1]

To a mixture of (S)-1-((6-(3′-(3-(((tert-butoxycarbonyl)(((S)-5-oxopyrrolidin-2-yl)methyl) amino)methyl)-4-oxo-4H-pyrido[1,2-a]pyrimidin-8-yl)-2,2′-dichloro-[1,1′-biphenyl]-3-yl)-2-methoxypyridin-3-yl)methyl)pyrrolidine-3-carboxylic acid (0.5 g, 0.6 mmol) in DCM (5 mL) was added trifluoroacetic acid (6 mL, 81 mmol). The mixture was stirred at r.t. for 0.5 h under N₂, then concentrated down. The residue was purified by reverse phase HPLC to afford (S)-1-((6-(2,2′-dichloro-3′-(4-oxo-3-(((((S)-5-oxopyrrolidin-2-yl)methyl)amino)methyl)-4H-pyrido[1,2-a]pyrimidin-8-yl)-[1,1′-biphenyl]-3-yl)-2-methoxypyridin-3-yl)methyl)pyrrolidine-3-carboxylic acid as a white solid (114.8 mg, 25% yield, m/z: 727 [M+H]⁺ observed, Method K). ¹H NMR (400 MHz, Methanol-d₄): δ9.18 (d, J=7.6 Hz, 1H), 8.48 (s, 1H), 7.93 (d, J=7.2 Hz, 1H), 7.83 (d, J=1.2 Hz, 1H), 7.71 (dd, J=7.2 Hz, J=1.6 Hz, 1H), 7.65-7.54 (m, 5H), 7.45 (dd, J=7.2 Hz, J=1.6 Hz, 1H), 7.38 (d, J=7.6 Hz, 1H), 4.45 (s, 2H), 4.14-4.06 (m, 5H), 3.98-3.95 (m, 1H), 3.65-3.61 (m, 1H), 3.48-3.37 (m, 3H), 3.17-3.15 (m, 1H), 3.00-2.92 (m, 2H), 2.41-2.27 (m, 5H), 1.93-1.84 (m, 1H).

### Example 174: 8-(2,2′-Dichloro-3′-(5-((((3R,4S)-3-hydroxytetrahydro-2H-pyran-4-yl)amino)methyl)-6-methoxypyridin-2-yl)-[1,1′-biphenyl]-3-yl)-3-(((((S)-5-oxopyrrolidin-2-yl)methyl)amino)methyl)-4H-pyrido[1,2-a]pyrimidin-4-one

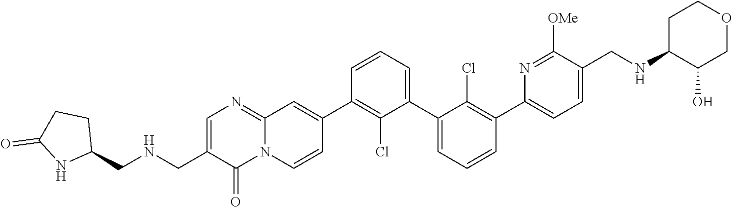 [SMILES: COc1nc(-c2cccc(-c3cccc(-c4ccn5c(=O)c(CNC[C@@H]6CCC(=O)N6)cnc5c4)c3Cl)c2Cl)ccc1CN[C@H]1CCOC[C@@H]1O]

This compound was prepared in a similar fashion to Example 118. using (3R,4S)-4-aminotetrahydro-2H-pyran-3-ol and (S)-tert-butyl ((8-(2,2′-dichloro-3′-(5-formyl-6-methoxypyridin-2-yl)-[1,1′-biphenyl]-3-yl)-4-oxo-4H-pyrido[1,2-a]pyrimidin-3-yl)methyl)((5-oxopyrrolidin-2-yl)methyl)carbamate. MS: m/z found 729 [M+H]+, (Method A). ¹H NMR (400 MHz, Methanol-d₄) δ 9.12 (d, J=8 Hz, 1H), 8.41 (s, 1H), 7.76 (s, 1H), 7.72 (d, J=7.6 Hz, 1H), 7.66 (d, J=8 Hz, 1H), 7.61-7.53 (m, 2H), 7.52-7.48 (m, 3H), 7.39 (dd, J=8 Hz, J=1.6 Hz, 1H), 7.24 (d, J=7.2 Hz, 1H), 4.02 (s, 3H), 3.93-3.78 (m, 7H), 3.42-3.33 (m, 2H), 3.05 (t, J=10.4 Hz, 1H), 2.77-2.65 (m, 2H), 2.56-2.50 (m, 1H), 2.35-2.24 (m, 3H), 2.11-2.06 (m, 1H), 1.84-1.79 (m, 1H), 1.46-1.43 (m, 1H).

### Example 175: 8-(2,2′-Dichloro-3′-(5-(((S)-3-hydroxypyrrolidin-1-yl)methyl)-6-methoxy pyridin-2-yl)-[1,1′-biphenyl]-3-yl)-3-(((((S)-5-oxopyrrolidin-2-yl)methyl)amino)methyl)-4H-pyrido[1,2-a]pyrimidin-4-one

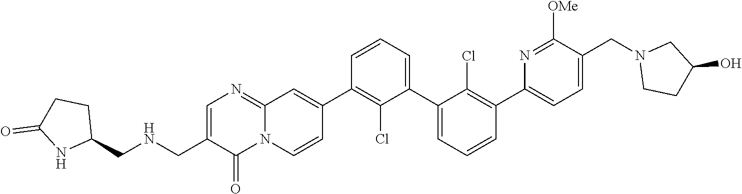 [SMILES: COc1nc(-c2cccc(-c3cccc(-c4ccn5c(=O)c(CNC[C@@H]6CCC(=O)N6)cnc5c4)c3Cl)c2Cl)ccc1CN1CC[C@H](O)C1]

This compound was prepared in a similar fashion to Example 118, using (3S)-pyrrolidin-3-ol and (S)-tert-butyl ((8-(2,2′-dichloro-3′-(5-formyl-6-methoxypyridin-2-yl)-[1,1′-biphenyl]-3-yl)-4-oxo-4H-pyrido[1,2-a]pyrimidin-3-yl)methyl)((5-oxopyrrolidin-2-yl)methyl)carbamate. MS: m/z found 699 [M+H]⁺, (Method C). ¹H NMR (400 MHz, Methanol-d₄): δ 9.19 (d, J=7.6 Hz, 1H), 8.50 (s, 1H), 7.92 (d, J=7.6 Hz, 1H), 7.85 (s, 1H), 7.68-7.62 (m, 6H), 7.46-7.45 (m, 1H), 7.38 (d, J=7.6 Hz, 1H), 4.58 (s, 2H), 4.47 (s, 2H), 4.23 (s, 2H), 4.09 (s, 3H), 4.01 (s, 1H), 3.61 (s, 1H), 3.48 (s, 1H), 3.11 (s, 2H), 2.41-2.32 (m, 5H), 2.09 (s, 1H), 1.88 (s, 1H).

### Example 197: 8-(2,2′-Dichloro-3′-(5-((((S)-2-hydroxypropyl)amino)methyl)-6-methoxy pyridin-2-yl)-[1,1′-biphenyl]-3-yl)-3-(((((S)-5-oxopyrrolidin-2-yl)methyl)amino)methyl)-4H-pyrido[1,2-a]pyrimidin-4-one

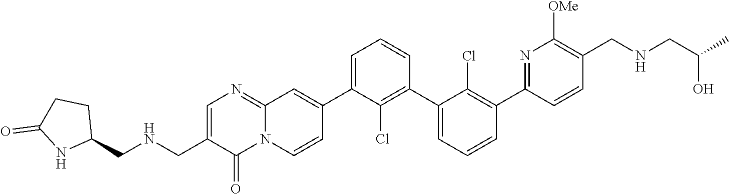 [SMILES: COc1nc(-c2cccc(-c3cccc(-c4ccn5c(=O)c(CNC[C@@H]6CCC(=O)N6)cnc5c4)c3Cl)c2Cl)ccc1CNC[C@H](C)O]

This compound was prepared in a similar fashion to Example 118, using (5S)-5-(aminomethyl)pyrrolidin-2-one and (S)-tert-butyl ((8-(2,2′-dichloro-3′-(5-formyl-6-methoxypyridin-2-yl)-[1,1′-biphenyl]-3-yl)-4-oxo-4H-pyrido[1,2-a]pyrimidin-3-yl)methyl)((5-oxopyrrolidin-2-yl)methyl)carbamate. MS: m/z found 687 [M+H]⁺, (Method C). ¹H NMR (400 MHz, Methanol-d₄): δ 9.18 (d, J=7.6 Hz, 1H), 8.49 (s, 1H), 7.85 (m, 2H), 7.68 (dd, J=7.6 Hz, 1H), 7.63 (m, 3H), 7.54 (m, 2H), 7.44 (m, 1H), 7.36 (d, J=7.2 Hz, 1H), 4.30 (s, 2H), 4.20 (s, 2H), 4.07 (m, 4H), 4.00 (s, 1H), 3.13 (m, 3H), 2.91 (m, 1H), 2.38 (m, 3H), 1.88 (m, 1H), 1.25 (d, J=6.4 Hz, 3H).

### Example 198: 8-(2,2′-Dichloro-3′-(5-((((R)-2-hydroxypropyl)amino)methyl)-6-methoxy pyridin-2-yl)-[1,1′-biphenyl]-3-yl)-3-(((((S)-5-oxopyrrolidin-2-yl)methyl)amino)methyl)-4H-pyrido[1,2-a]pyrimidin-4-one

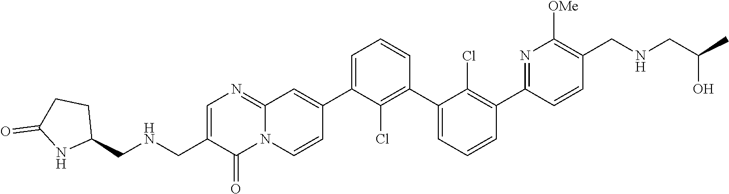 [SMILES: COc1nc(-c2cccc(-c3cccc(-c4ccn5c(=O)c(CNC[C@@H]6CCC(=O)N6)cnc5c4)c3Cl)c2Cl)ccc1CNC[C@@H](C)O]

### tert-Butyl ((6-(2,2′-dichloro-3′-(4-oxo-3-(((((S)-5-oxopyrrolidin-2-yl)methyl)amino)methyl)-4H-pyrido[1,2-a]pyrimidin-8-yl)-[1,1′-biphenyl]-3-yl)-2-methoxypyridin-3-yl)methyl)((R)-2-hydroxypropyl)carbamate

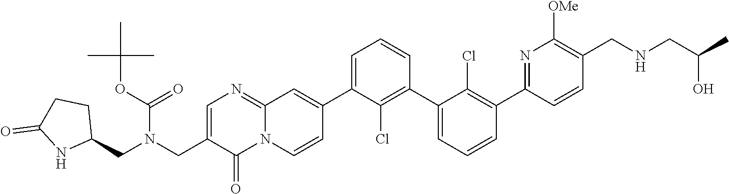 [SMILES: COc1nc(-c2cccc(-c3cccc(-c4ccn5c(=O)c(CN(C[C@@H]6CCC(=O)N6)C(=O)OC(C)(C)C)cnc5c4)c3Cl)c2Cl)ccc1CNC[C@@H](C)O]

To a mixture of tert-butyl N-[[6-[2-chloro-3-[2-chloro-3-(3-formyl-4-oxo-pyrido[1,2-a]pyrimidin-8-yl)phenyl]phenyl]-2-methoxy-3-pyridyl]methyl]-N-[(2R)-2-hydroxypropyl]carbamate (0.15 g, 0.21 mmol), (5S)-5-(aminomethyl)pyrrolidin-2-one (0.03 g, 0.32 mmol) in DCM (2 mL) was added sodium acetate (0.05 g, 0.65 mmol). The mixture was stirred at r.t. for 1 h. Sodium triacetoxyborohydride (0.27 g, 1.13 mmol) was added to the mixture and stirred for 1 h. The reaction mixture was filtered. The filtrate was used directly for the next step directly. (m/z: 787 [M]+ observed Method F).

### 8-(2,2′-Dichloro-3′-(5-((((R)-2-hydroxypropyl)amino)methyl)-6-methoxypyridin-2-yl)-[1,1′-biphenyl]-3-yl)-3-(((((S)-5-oxopyrrolidin-2-yl)methyl)amino)methyl)-4H-pyrido[1,2-a]pyrimidin-4-one

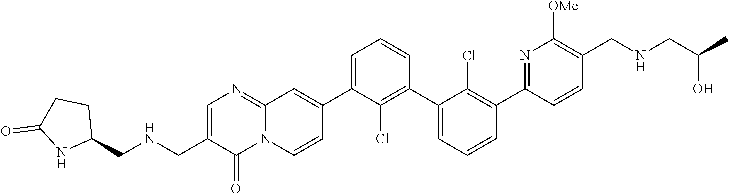 [SMILES: COc1nc(-c2cccc(-c3cccc(-c4ccn5c(=O)c(CNC[C@@H]6CCC(=O)N6)cnc5c4)c3Cl)c2Cl)ccc1CNC[C@@H](C)O]

To a solution of tert-butyl N-[[6-[2-chloro-3-[2-chloro-3-[4-oxo-3-[[[(2S)-5-oxopyrrolidin-2-yl]methylamino]methyl]pyrido[1,2-a]pyrimidin-8-yl]phenyl]phenyl]-2-methoxy-3-pyridyl]methyl]-N-[(2R)-2-hydroxypropyl]carbamate in DCM (2 mL) was added TFA (3 mL), and the reaction was stirred at r.t. for 1 h. The reaction was concentrated under reduced pressure. The residue was purified by reverse phase HPLC to afford 8-(2,2′-dichloro-3′-(5-((((R)-2-hydroxypropyl)amino)methyl)-6-methoxypyridin-2-yl)-[1,1′-biphenyl]-3-yl)-3-(((((S)-5-oxopyrrolidin-2-yl)methyl)amino)methyl)-4H-pyrido[1,2-a]pyrimidin-4-one (86 mg, 57% yield for two steps, as formic acid salt, m/z: 687 [M+H]+ observed, method C) as white solid.)¹H NMR (400 MHz, Methanol-d₄): δ 9.18 (d, J=7.2 Hz, 1H), 8.49 (s, 1H), 7.85 (m, 2H), 7.65 (m, 4H), 7.53 (m, 2H), 7.44 (d, J=6.0 Hz 1H), 7.36 (d, J=8.0 Hz, 1H), 4.30 (s, 2H), 4.19 (s, 2H), 4.09 (s, 4H), 3.99 (s, 1H), 3.12 (m, 3H), 2.91 (m, 1H), 2.39 (m, 3H), 1.88 (d, J=7.6 Hz, 1H), 1.25 (d, J=6.4 Hz, 3H).

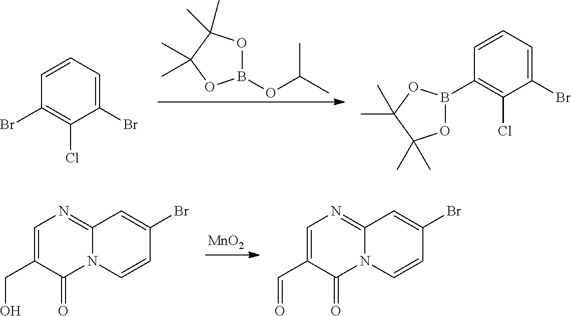

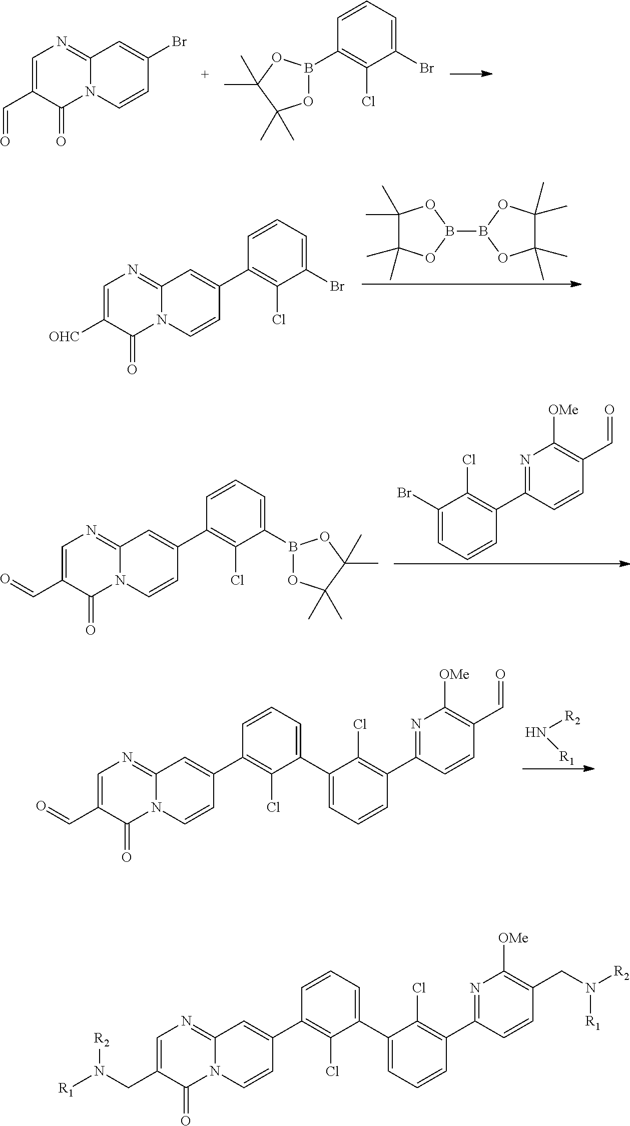

### Example 165: 8-(2,2′-Dichloro-3′-(5-(((S)-3-hydroxypyrrolidin-1-yl)methyl)-6-methoxypyridin-2-yl)-[1,1′-biphenyl]-3-yl)-3-(((S)-3-hydroxypyrrolidin-1-yl)methyl)-4H-pyrido[1,2-a]pyrimidin-4-one

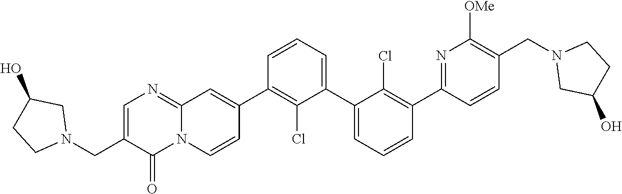 [SMILES: COc1nc(-c2cccc(-c3cccc(-c4ccn5c(=O)c(CN6CC[C@@H](O)C6)cnc5c4)c3Cl)c2Cl)ccc1CN1CC[C@@H](O)C1]

### 2-(3-Bromo-2-chlorophenyl)-4,4,5,5-tetramethyl-1,3,2-dioxaborolane

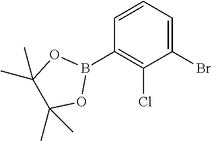 [SMILES: CC1(C)OB(c2cccc(Br)c2Cl)OC1(C)C]

A mixture of lithium chloro(isopropyl)magnesium chloride (110 mL, 144 mmol, 1.3 M) in THE (180 mL) was cooled to −20° C., then 1,3-dibromo-2-chlorobenzene (30 g, 110 mmol) was added to the mixture and stirred at −20° C. for 1 h under N₂, then 2-isopropoxy-4,4,5,5-tetramethyl-1,3,2-dioxaborolane (29 mL, 144 mmol) was added to the mixture and stirred at −10° C. for 0.5 h. The reaction was warmed to rt and stirred for 4 h under N₂. The mixture was cooled to 0° C., water (300 mL) was added to the mixture and stirred at rt for 0.5 h. The mixture was filtered and concentrated to remove most of the THF, then was extracted with EtOAc (2×400 mL). The combined organic phase was dried over anhydrous sulfate, filtered and concentrated. The residue was purified by normal phase SiO₂ chromatography (0-10% EtOAc/petroleum ether) to afford 2-(3-bromo-2-chlorophenyl)-4,4,5,5-tetramethyl-1,3,2-dioxaborolane as a white solid (30 g, 58% yield). ¹H NMR (400 MHz, CDCl₃): δ 7.59 (dd, J₁=1.6 Hz, J₂=8 Hz, 1H), 7.52 (dd, J₁=1.6 Hz, J₂=7.2 Hz, 1H), 7.02 (t, J=7.6 Hz, 1H), 1.30 (s, 12H).

### 8-Bromo-4-oxo-4H-pyrido[1,2-a]pyrimidine-3-carbaldehyde

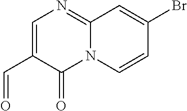 [SMILES: O=Cc1cnc2cc(Br)ccn2c1=O]

To a solution of 8-bromo-3-(hydroxymethyl)-4H-pyrido[1,2-a]pyrimidin-4-one (11 g, 43.13 mmol) in CHCl₃ (450 mL) was added MnO₂ (26.05 g, 299.67 mmol). The mixture was stirred at 60° C. for 15 hr under N₂, then filtered through celite and the filter cake was washed with THE (10×80 mL). The filtrate was concentrated, and the crude product was triturated with EtOAc/petroleum ether (1/2, 60 mL) at 15° C. for 0.5 hr. The brown solid was collected by filtration to afford 8-bromo-4-oxo-4H-pyrido[1,2-a]pyrimidine-3-carbaldehyde (7.3 g, 67% yield) as a yellow solid. ¹H NMR (400 MHz, DMSO-d₆): δ 10.14 (s, 1H), 9.05 (d, J=7.2 Hz, 1H), 8.73 (s, 1H), 8.26 (s, 1H), 7.79 (d, J=7.6 Hz, 1H).

### 8-(3-Bromo-2-chlorophenyl)-4-oxo-4H-pyrido[1,2-a]pyrimidine-3-carbaldehyde

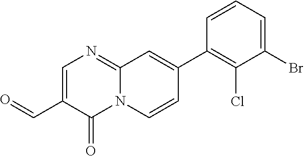 [SMILES: O=Cc1cnc2cc(-c3cccc(Br)c3Cl)ccn2c1=O]

To a mixture of 8-bromo-4-oxo-4H-pyrido[1,2-a]pyrimidine-3-carbaldehyde (7 g, 27.7 mmol), 2-(3-bromo-2-chlorophenyl)-4,4,5,5-tetramethyl-1,3,2-dioxaborolane (7 g, 22.1 mmol) in THF/H₂O mixture (5:1, 120 mL), was added potassium carbonate (11.5 g, 83 mmol), and [1,1-Bis(diphenylphosphino)ferrocene]palladium(II) chloride dichloromethane complex (2.3 g, 2.8 mmol). The reaction was stirred at 80° C. for 3 hr under N₂, then was concentrated. The residue was triturated with EtOAc (50 mL) at 15° C. for 0.5 hr to afford 8-(3-bromo-2-chlorophenyl)-4-oxo-4H-pyrido[1,2-a]pyrimidine-3-carbaldehyde (7.5 g, 74% yield) as a brown solid. ¹H NMR (400 MHz, DMSO-d₆): δ 10.20 (s, 1H), 9.27 (d, J=6.4 Hz, 1H), 8.79 (s, 1H), 8.02-7.97 (m, 2H), 7.76 (d, J=6.8 Hz, 1H), 7.63-7.61 (m, 1H), 7.51-7.49 (m, 1H).

### 8-(2-Chloro-3-(4,4,5,5-tetramethyl-1,3,2-dioxaborolan-2-yl)phenyl)-4-oxo-4H-pyrido[1,2-a]pyrimidine-3-carbaldehyde

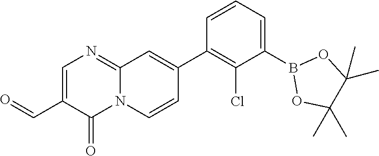 [SMILES: CC1(C)OB(c2cccc(-c3ccn4c(=O)c(C=O)cnc4c3)c2Cl)OC1(C)C]

To a mixture of 8-(3-bromo-2-chlorophenyl)-4-oxo-4H-pyrido[1,2-a]pyrimidine-3-carbaldehyde (7.5 g, 20.6 mmol), 4,4,4′,4′,5,5,5′,5′-octamethyl-2,2′-bi(1,3,2-dioxaborolane) (10.5 g, 41.2 mmol) in dioxane/THF mixture(1:1, 200 mL), was added potassium acetate (6.1 g, 61.9 mmol), and [1,1-Bis(diphenylphosphino)ferrocene]palladium(II) chloride dichloromethane complex (1.7 g, 2.1 mmol). The reaction was stirred at 110° C. for 12 hr under N₂, then was concentrated. The residue was purified by normal phase SiO₂ chromatography (0-60% EtOAc/petroleum ether) to afford 8-(2-chloro-3-(4,4,5,5-tetramethyl-1,3,2-dioxaborolan-2-yl)phenyl)-4-oxo-4H-pyrido[1,2-a]pyrimidine-3-carbaldehyde (6.5 g, 57% yield, m/z: 411 [M+H]⁺ observed, Method Q) as a yellow solid. ¹H NMR (400 MHz, DMSO-d₆): δ 10.19 (s, 1H), 9.24 (d, J=7.2 Hz, 1H), 8.77 (s, 1H), 7.96-7.93 (m, 1H), 7.78-7.73 (m, 2H), 7.68-7.66 (m, 1H), 7.55-7.53 (m, 1H), 1.33 (s, 12H).

### 8-(2,2′-Dichloro-3′-(5-formyl-6-methoxypyridin-2-yl)-[1,1′-biphenyl]-3-yl)-4-oxo-4H-pyrido[1,2-a]pyrimidine-3-carbaldehyde

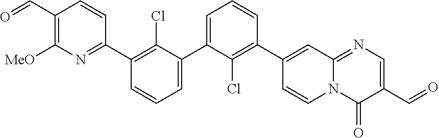 [SMILES: COc1nc(-c2cccc(-c3cccc(-c4ccn5c(=O)c(C=O)cnc5c4)c3Cl)c2Cl)ccc1C=O]

To a mixture of 6-(3-bromo-2-chlorophenyl)-2-methoxynicotinaldehyde (2 g, 6.12 mmol), 8-(2-chloro-3-(4,4,5,5-tetramethyl-1,3,2-dioxaborolan-2-yl)phenyl)-4-oxo-4H-pyrido[1,2-a]pyrimidine-3-carbaldehyde (3.77 g, 9.19 mmol) in dioxane/H₂O mixture (5:1, 48 mL) was added potassium phosphate (2.6 g, 12.2 mmol), and dichloro[1,1′his(di-isopropylphosphino) ferrocene]palladium(II) (0.4 g, 0.61 mmol), and then the mixture was stirred at 110° C. for 1 h under N₂. The mixture was filtered and concentrated. The residue was purified by normal phase SiO₂ chromatography (0-60% EtOAc/petroleum ether), then triturated with EtOAc/petroleum ether (1:1, 12 mL) at 15° C. for 0.5 h to afford 8-(2,2′-dichloro-3′-(5-formyl-6-methoxypyridin-2-yl)-[1,1′-biphenyl]-3-yl)-4-oxo-4H-pyrido[1,2-a]pyrimidine-3-carbaldehyde (1.8 g, 48% yield, m/z: 530 [M+H]⁺ observed, Method AD) as a yellow solid. ¹H NMR (400 MHz, DMSO-d₆): δ 10.28 (s, 1H), 10.18 (s, 1H), 9.26 (d, J=7.2, 1H), 8.77 (s, 1H), 8.20 (d, J=8, 1H), 8.02 (s, 1H), 7.78-7.74 (m, 2H), 7.70-7.61 (m, 4H), 7.56-7.55 (m, 1H), 7.48-7.46 (m, 1H), 4.04 (s, 3H).

### 8-(2,2′-Dichloro-3′-(5-(((S)-3-hydroxypyrrolidin-1-yl)methyl)-6-methoxypyridin-2-yl)-[1,1′-biphenyl]-3-yl)-3-(((S)-3-hydroxypyrrolidin-1-yl)methyl)-4H-pyrido[1,2-a]pyrimidin-4-one

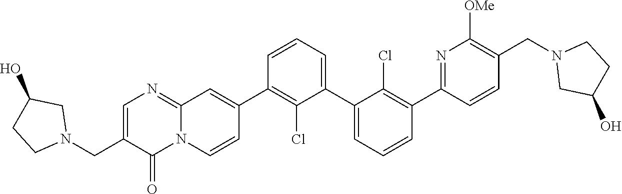 [SMILES: COc1nc(-c2cccc(-c3cccc(-c4ccn5c(=O)c(CN6CC[C@@H](O)C6)cnc5c4)c3Cl)c2Cl)ccc1CN1CC[C@@H](O)C1]

To a mixture of 8-(2,2′-dichloro-3′-(5-formyl-6-methoxypyridin-2-yl)-[1,1′-biphenyl]-3-yl)-4-oxo-4H-pyrido[1,2-a]pyrimidine-3-carbaldehyde (250 mg, 0.47 mmol), (S)-pyrrolidin-3-ol (411 mg, 4.71 mmol), sodium acetate (232 mg, 2.83 mmol), 4 Å molecular sieve (80 mg) was added DCM (20 mL), and reaction was stirred at r.t. for 2 h under N₂. Then sodium triacetoxyborohydride (599 mg, 2.83 mmol) was added the reaction, which was then stirred at r.t. for 0.5 hr under N₂. The reaction was filtered and concentrated. The residue was purified by reverse phase HPLC to afford 8-(2,2′-dichloro-3′-(5-(((S)-3-hydroxypyrrolidin-1-yl)methyl)-6-methoxypyridin-2-yl)-[1,1′-biphenyl]-3-yl)-3-(((S)-3-hydroxypyrrolidin-1-yl)methyl)-4H-pyrido[1,2-a]pyrimidin-4-one (56.5 mg, 18% yield, m/z: 672 [M+H]⁺ observed, Method A) as a white solid. ¹H NMR (400 MHz, Methanol-d₄): δ 9.12 (d, J=7.6 Hz, 1H), 8.42 (s, 1H), 7.77-7.75 (m, 2H), 7.64-7.58 (m, 3H), 7.51-7.49 (m, 3H), 7.39-7.38 (m, 1H), 7.23 (d, J=7.2 Hz, 1H), 4.37-4.33 (m, 2H), 3.98 (s, 3H), 3.74-3.71 (m, 2H), 3.70-3.66 (m, 2H), 2.90-2.82 (m, 4H), 2.64-2.58 (m, 4H), 2.18-2.13 (m, 2H), 1.76-1.70 (m, 2H).

### Example 185: 8-(2,2′-Dichloro-3′-(5-((((S)-2-hydroxypropyl)amino)methyl)-6-methoxypyridin-2-yl)-[1,1′-biphenyl]-3-yl)-3-((((S)-2-hydroxypropyl)amino)methyl)-4H-pyrido[1,2-a]pyrimidin-4-one

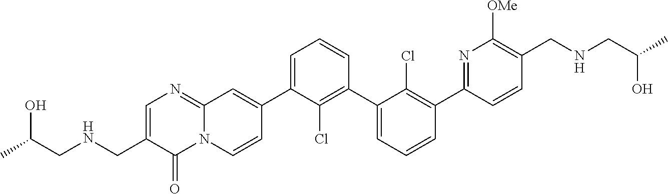 [SMILES: COc1nc(-c2cccc(-c3cccc(-c4ccn5c(=O)c(CNC[C@H](C)O)cnc5c4)c3Cl)c2Cl)ccc1CNC[C@H](C)O]

This compound was prepared in a similar fashion to Example 165, using (S)-1-aminopropan-2-ol and 8-(2,2′-dichloro-3′-(5-formyl-6-methoxypyridin-2-yl)-[1,1′-biphenyl]-3-yl)-4-oxo-4H-pyrido[1,2-a]pyrimidine-3-carbaldehyde. MS: m/z found 648 [M+H]⁺, (Method C). ¹H NMR (400 MHz, Methanol-d₄): δ 9.14 (d, J=7.2 Hz, 1H), 8.41 (s, 1H), 7.78 (s, 1H), 7.73 (d, J=7.6 Hz, 1H), 7.66-7.61 (m, 3H), 7.55-7.52 (m, 3H), 7.42 (d, J=1.6 Hz, 1H), 7.26 (d, J=7.6 Hz, 1H), 4.03 (s, 3H), 3.93-3.81 (m, 6H), 2.67-2.55 (m, 4H), 1.18 (d, J=6.0 Hz, 6H).

### Example 186: 8-(2,2′-Dichloro-3′-(5-((((R)-2-hydroxypropyl)amino)methyl)-6-methoxypyridin-2-yl)-[1,1′-biphenyl]-3-yl)-3-((((R)-2-hydroxypropyl)amino)methyl)-4H-pyrido[1,2-a]pyrimidin-4-one

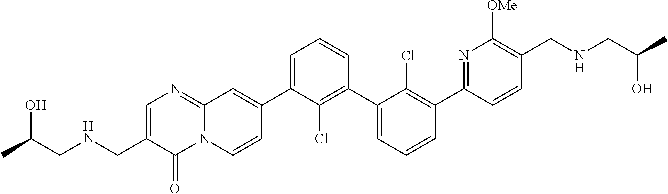 [SMILES: COc1nc(-c2cccc(-c3cccc(-c4ccn5c(=O)c(CNC[C@@H](C)O)cnc5c4)c3Cl)c2Cl)ccc1CNC[C@@H](C)O]

This compound was prepared in a similar fashion to Example 165, using (R)-1-aminopropan-2-ol and 8-(2,2′-dichloro-3′-(5-formyl-6-methoxypyridin-2-yl)-[1,1′-biphenyl]-3-yl)-4-oxo-4H-pyrido[1,2-a]pyrimidine-3-carbaldehyde. MS: m/z found 648 [M+H]⁺, (Method C). ¹H NMR (400 MHz, DMSO-d₆): δ 9.02 (d, J=7.2 Hz, 1H), 8.40 (s, 1H), 7.79 (d, J=9.2 Hz, 1H), 7.77 (s, 1H), 7.68-7.63 (m, 3H), 7.58-7.56 (m, 2H), 7.49-7.48 (m, 2H), 7.28 (d, J=7.2 Hz, 1H), 4.52 (d, J=4.4 Hz, 1H), 4.47 (d, J=4.8 Hz, 1H), 3.92 (s, 3H), 3.76-3.67 (m, 6H), 3.32 (s, 1H), 2.50-2.43 (m, 4H), 2.16 (s, 1H), 1.05 (dd, J=8.0 Hz, J=6.4 Hz, 6H).

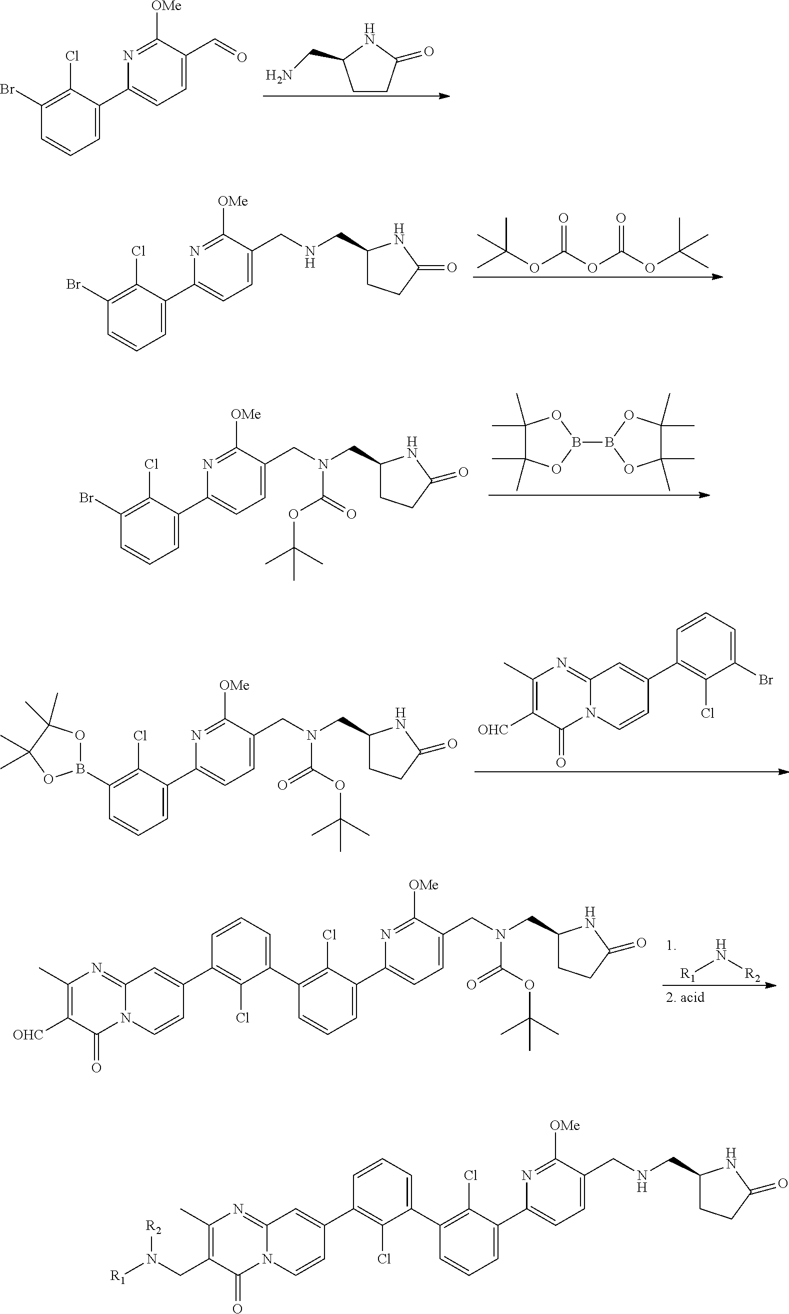

### Example 193: 8-(2,2′-Dichloro-3′-(6-methoxy-5-(((((S)-5-oxopyrrolidin-2-yl)methyl)amino) methyl)pyridin-2-yl)-[1,1′-biphenyl]-3-yl)-3-((((S)-2-hydroxypropyl)amino)methyl)-2-methyl-4H-pyrido[1,2-a]pyrimidin-4-one

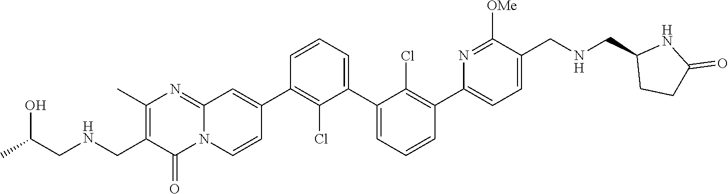 [SMILES: COc1nc(-c2cccc(-c3cccc(-c4ccn5c(=O)c(CNC[C@H](C)O)c(C)nc5c4)c3Cl)c2Cl)ccc1CNC[C@@H]1CCC(=O)N1]

### (S)-5-((((6-(3-Bromo-2-chlorophenyl)-2-methoxypyridin-3-yl)methyl)amino) methyl) pyrrolidin-2-one

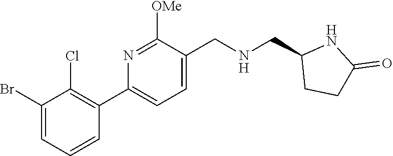 [SMILES: COc1nc(-c2cccc(Br)c2Cl)ccc1CNC[C@@H]1CCC(=O)N1]

To a solution of 6-(3-bromo-2-chlorophenyl)-2-methoxynicotinaldehyde (3 g, 9.19 mmol) and (S)-5-(aminomethyl)pyrrolidin-2-one HCl (2.08 g, 13.8 mmol) in DCM (50 mL) was added sodium acetate (1.51 g, 18.4 mmol). The mixture was stirred at r.t. for 0.5 h, then sodium triacetoxyborohydride (2.92 g, 13.8 mmol) added. The reaction was stirred at r.t. for 1.5 hr, giving the (S)-5-((((6-(3-bromo-2-chlorophenyl)-2-methoxypyridin-3-yl)methyl)amino)methyl)pyrrolidin-2-one (3.9 g, crude, m/z: 424 [M+H]⁺ observed (LCMS: Method A)) as yellow solution DCM.

### (S)-tert-Butyl ((6-(3-bromo-2-chlorophenyl)-2-methoxypyridin-3-yl)methyl)((5-oxopyrrolidin-2-yl)methyl)carbamate

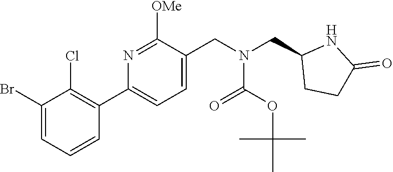 [SMILES: COc1nc(-c2cccc(Br)c2Cl)ccc1CN(C[C@@H]1CCC(=O)N1)C(=O)OC(C)(C)C]

To a solution of (S)-5-((((6-(3-bromo-2-chlorophenyl)-2-methoxypyridin-3-yl)methyl)amino)methyl)pyrrolidin-2-one (3.9 g, 9.18 mmol, 1 eq) in DCM (70 mL) was added triethylamine (2.56 mL, 18.4 mmol) and di-tert-butyl dicarbonate (4.01 g, 18.4 mmol). The mixture was stirred at r.t. for 12 hr. H₂O (50 mL) was added to the mixture, and it was then extracted with DCM (40 mL). The combined organic layers were dried over anhydrous sodium sulfate, filtered and concentrated. The residue was purified by normal phase SiO₂ chromatography (0-100% EtOAc/petroleum ether) to afford (S)-tert-butyl ((6-(3-bromo-2-chlorophenyl)-2-methoxypyridin-3-yl)methyl)((5-oxopyrrolidin-2-yl)methyl)carbamate (2 g, 42% yield for two steps) as a white solid. ¹H NMR (400 MHz, DMSO-d₆) δ 7.85 (d, J=6.4 Hz, 1H), 7.77 (s, 1H), 7.58 (dd, J=7.6 Hz, J=1.6 Hz, 1H), 7.39 (t, J=8 Hz, 1H), 7.24 (d, J=7.2 Hz, 1H), 4.47-4.34 (m, 2H), 3.91 (s, 3H), 3.78-3.76 (m, 1H), 3.31-3.26 (m, 2H), 2.18-2.06 (m, 3H), 1.74 (m, 1H), 1.40 (d, J=52.8 Hz, 9H).

### (S)-tert-Butyl ((6-(2-chloro-3-(4,4,5,5-tetramethyl-1,3,2-dioxaborolan-2-yl)phenyl)-2-methoxypyridin-3-yl)methyl)((5-oxopyrrolidin-2-yl)methyl)carbamate

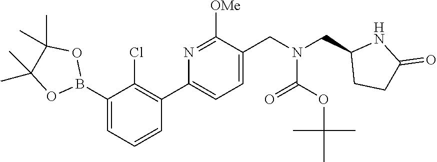 [SMILES: COc1nc(-c2cccc(B3OC(C)(C)C(C)(C)O3)c2Cl)ccc1CN(C[C@@H]1CCC(=O)N1)C(=O)OC(C)(C)C]

To a mixture (S)-tert-butyl ((6-(3-bromo-2-chlorophenyl)-2-methoxypyridin-3-yl)methyl)((5-oxopyrrolidin-2-yl)methyl)carbamate (1 g, 1.91 mmol, 1 eq) in dioxane (3 mL) was added 4,4,4′,4′,5,5,5′,5′-octamethyl-2,2′-bi(1,3,2-dioxaborolane) (1.45 g, 5.72 mmol), 1,1′-bis(diphenylphosphino)ferrocene-palladium(ii) dichloride dichloromethane complex (0.16 g, 0.19 mmol) and potassium acetate (0.56 g, 5.72 mmol). The reaction was stirred at 80° C. for 3 hr. The reaction mixture was filtered and concentrated under reduced pressure. The residue was purified by normal phase SiO₂ chromatography (0-100% EtOAc/petroleum ether) to afford (S)-tert-butyl ((6-(2-chloro-3-(4,4,5,5-tetramethyl-1,3,2-dioxaborolan-2-yl)phenyl)-2-methoxypyridin-3-yl)methyl)((5-oxopyrrolidin-2-yl)methyl)carbamate as a yellow solid (1 g, 65% yield, LCMS: 572 [M+H]⁺ observed LCMS method J).

### (S)-tert-Butyl ((6-(2,2′-dichloro-3′-(3-formyl-2-methyl-4-oxo-4H-pyrido[1,2-a]pyrimidin-8-yl)-[1,1′-biphenyl]-3-yl)-2-methoxypyridin-3-yl)methyl)((5-oxopyrrolidin-2-yl)methyl) carbamate

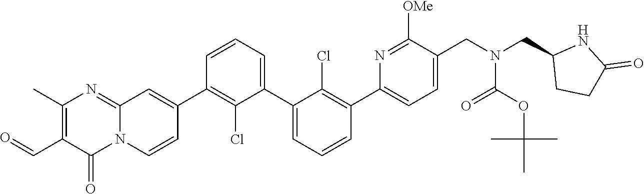 [SMILES: COc1nc(-c2cccc(-c3cccc(-c4ccn5c(=O)c(C=O)c(C)nc5c4)c3Cl)c2Cl)ccc1CN(C[C@@H]1CCC(=O)N1)C(=O)OC(C)(C)C]

To a mixture (S)-tert-butyl ((6-(2-chloro-3-(4,4,5,5-tetramethyl-1,3,2-dioxaborolan-2-yl)phenyl)-2-methoxypyridin-3-yl)methyl)((5-oxopyrrolidin-2-yl)methyl)carbamate (0.5 g, 0.87 mmol) in dioxane/H₂O (4:1, (2.5 mL) was added 8-(3-bromo-2-chlorophenyl)-2-methyl-4-oxo-4H-pyrido[1,2-a]pyrimidine-3-carbaldehyde (0.56 g, 1.49 mmol), [1,1′-bis(di-tert-butylphosphino)ferrocene]dichloropalladium(II) (0.06 g, 0.09 mmol) and potassium phosphate (0.56 g, 2.62 mmol). The reaction was stirred at 110° C. for 0.5 hr. The reaction mixture was filtered and concentrated under reduced pressure. The residue was purified by normal phase SiO₂ chromatography (0-100% EtOAc/petroleum ether) to afford (S)-tert-butyl ((6-(2,2′-dichloro-3′-(3-formyl-2-methyl-4-oxo-4H-pyrido[1,2-a]pyrimidin-8-yl)-[1,1′-biphenyl]-3-yl)-2-methoxypyridin-3-yl)methyl)((5-oxopyrrolidin-2-yl)methyl)carbamate as a yellow solid (0.34 g, 52% yield, LCMS: 742 [M+H]⁺ observed LCMS method J).

### tert-Butyl ((6-(2,2′-dichloro-3′-(3-((((S)-2-hydroxypropyl)amino)methyl)-2-methyl-4-oxo-4H-pyrido[1,2-a]pyrimidin-8-yl)-[1,1′-biphenyl]-3-yl)-2-methoxypyridin-3-yl)methyl)(((S)-5-oxopyrrolidin-2-yl)methyl)carbamate

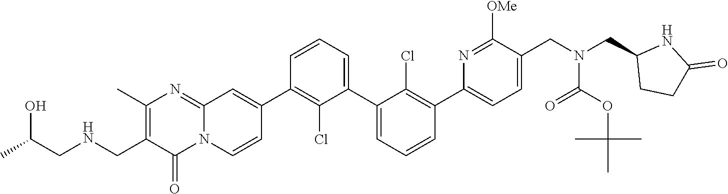 [SMILES: COc1nc(-c2cccc(-c3cccc(-c4ccn5c(=O)c(CNC[C@H](C)O)c(C)nc5c4)c3Cl)c2Cl)ccc1CN(C[C@@H]1CCC(=O)N1)C(=O)OC(C)(C)C]

To a mixture (S)-tert-butyl ((6-(2,2′-dichloro-3′-(3-formyl-2-methyl-4-oxo-4H-pyrido[1,2-a]pyrimidin-8-yl)-[1,1′-biphenyl]-3-yl)-2-methoxypyridin-3-yl)methyl)((5-oxopyrrolidin-2-yl)methyl)carbamate (0.3 g, 0.4 mmol) in DCM/MeOH (1:1, 4 mL) was added (S)-1-aminopropan-2-ol (0.18 g, 2.42 mmol), sodium acetate (0.2 g, 2.42 mmol). The reaction was stirred at r.t. for 2 hr. Sodium cyanoborohydride (0.08 g, 1.21 mmol) was added, then mixture was stirred at r.t. for 1 hr. The reaction mixture was filtered and concentrated under reduced pressure to give tert-butyl ((6-(2,2′-dichloro-3′-(3-((((S)-2-hydroxypropyl)amino)methyl)-2-methyl-4-oxo-4H-pyrido[1,2-a]pyrimidin-8-yl)-[1,1′-biphenyl]-3-yl)-2-methoxypyridin-3-yl)methyl)(((S)-5-oxopyrrolidin-2-yl)methyl)carbamate as a yellow solid (0.4 g, crude, LCMS: 801 [M+H]⁺ observed LCMS method J)).

### 8-(2,2′-Dichloro-3′-(6-methoxy-5-(((((S)-5-oxopyrrolidin-2-yl)methyl) amino)methyl) pyridin-2-yl)-[1,1′-biphenyl]-3-yl)-3-((((S)-2-hydroxypropyl)amino)methyl)-2-methyl-4H-pyrido[1,2-a]pyrimidin-4-one

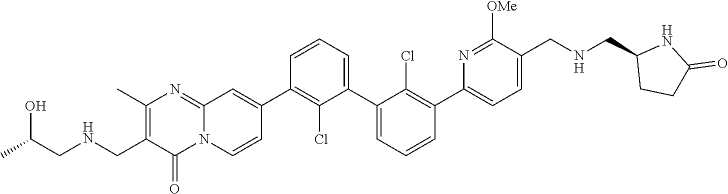 [SMILES: COc1nc(-c2cccc(-c3cccc(-c4ccn5c(=O)c(CNC[C@H](C)O)c(C)nc5c4)c3Cl)c2Cl)ccc1CNC[C@@H]1CCC(=O)N1]

A mixture tert-butyl ((6-(2,2′-dichloro-3′-(3-((((S)-2-hydroxypropyl)amino)methyl)-2-methyl-4-oxo-4H-pyrido[1,2-a]pyrimidin-8-yl)-[1,1′-biphenyl]-3-yl)-2-methoxypyridin-3-yl)methyl)(((S)-5-oxopyrrolidin-2-yl)methyl)carbamate (0.4 g, 0.5 mmol) in DCM/TFA (1:2.15 mL) was stirred at 25° C. for 2 hr. The reaction mixture was filtered and concentrated under reduced pressure to give a residue. The residue was purified by reverse phase HPLC to afford 8-(2,2′-dichloro-3′-(6-methoxy-5-(((((S)-5-oxopyrrolidin-2-yl)methyl)amino)methyl)pyridin-2-yl)-[1,1′-biphenyl]-3-yl)-3-((((S)-2-hydroxypropyl)amino)methyl)-2-methyl-4H-pyrido[1,2-a]pyrimidin-4-one as a white solid (42.3 mg, 12% yield, MS: 701 [M+H]⁺ observed MS method A). ¹H NMR (400 MHz, Methanol-d₄): δ 9.06 (d, J=7.6 Hz, 1H), 7.75 (d, J=7.6 Hz, 1H), 7.71 (d, J=1.6 Hz, 1H), 7.67 (dd, J=1.6 Hz, J=7.6 Hz, 1H), 7.64-7.59 (m, 2H), 7.54-7.48 (m, 3H), 7.41 (dd, J=1.6 Hz, J=7.6 Hz, 1H), 7.25 (d, J=7.6 Hz, 1H), 4.04 (s, 3H), 4.02-3.84 (m, 6H), 2.73-2.69 (m, 3H), 2.63-2.60 (m, 4H), 2.38-2.29 (m, 3H), 1.86-1.81 (m, 1H), 1.18 (d, J=7.4 Hz, 3H).

### Example 204: 8-(2,2′-Dichloro-3′-(6-methoxy-5-(((((S)-5-oxopyrrolidin-2-yl)methyl) amino)methyl)pyridin-2-yl)-[1,1′-biphenyl]-3-yl)-3-((((1r,3r)-3-hydroxy-3-methylcyclobutyl)amino)methyl)-2-methyl-4H-pyrido[1,2-a]pyrimidin-4-one

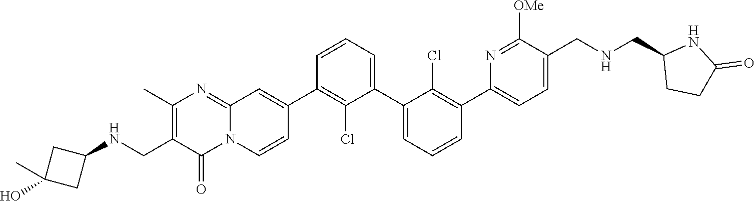 [SMILES: COc1nc(-c2cccc(-c3cccc(-c4ccn5c(=O)c(CN[C@H]6C[C@](C)(O)C6)c(C)nc5c4)c3Cl)c2Cl)ccc1CNC[C@@H]1CCC(=O)N1]

This compound was prepared in a similar fashion to Example 193, using (1r,3r)-3-amino-1-methylcyclobutan-1-ol and tert-butyl (S)-((6-(2,2′-dichloro-3′-(3-formyl-2-methyl-4-oxo-4H-pyrido[1,2-a]pyrimidin-8-yl)-[1,1′-biphenyl]-3-yl)-2-methoxypyridin-3-yl)methyl)((5-oxopyrrolidin-2-yl)methyl)carbamate. MS: m/z found 727 [M+H]⁺ (Method A). ¹H NMR (400 MHz, Methanol-d₄): δ 9.12 (d, J=7.6 Hz, 1H), 7.84 (d, J=7.6 Hz, 1H), 7.79 (s, 1H), 7.68-7.53 (m, 6H), 7.45-7.43 (m, 1H), 7.33 (d, J=7.2 Hz, 1H), 4.25 (s, 2H), 4.13-4.12 (m, 2H), 4.08 (s, 3H), 4.06-3.98 (m, 2H), 3.03-3.00 (m, 2H), 2.64 (s, 3H), 3.51-2.31 (s, 7H), 1.92-1.88 (m, 1H), 1.43 (s, 3H).

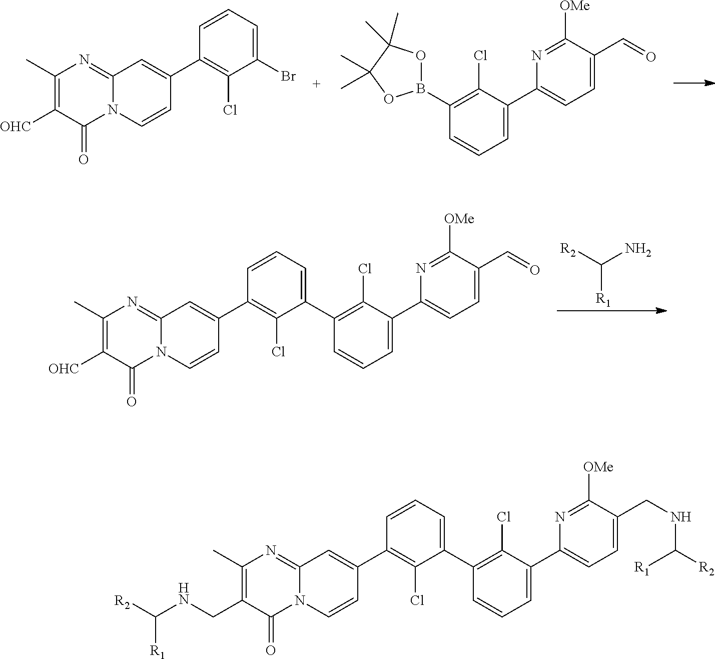

### Example 205: 8-(2,2′-Dichloro-3′-(5-((((1r,3r)-3-hydroxy-3-methylcyclobutyl)amino)methyl)-6-methoxypyridin-2-yl)-[1,1′-biphenyl]-3-yl)-3-((((1r,3r)-3-hydroxy-3-methylcyclobutyl) amino)methyl)-2-methyl-4H-pyrido[1,2-a]pyrimidin-4-one

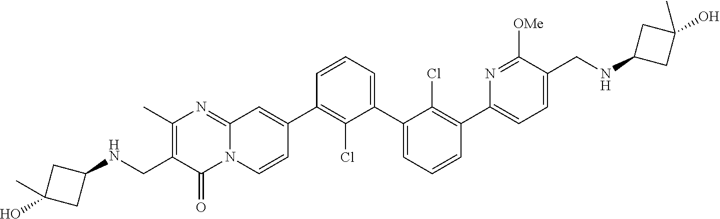 [SMILES: COc1nc(-c2cccc(-c3cccc(-c4ccn5c(=O)c(CN[C@H]6C[C@](C)(O)C6)c(C)nc5c4)c3Cl)c2Cl)ccc1CN[C@H]1C[C@](C)(O)C1]

### 8-(2,2′-Dichloro-3′-(5-formyl-6-methoxypyridin-2-yl)-[1,1′-biphenyl]-3-yl)-2-methyl-4-oxo-4H-pyrido[1,2-a]pyrimidine-3-carbaldehyde

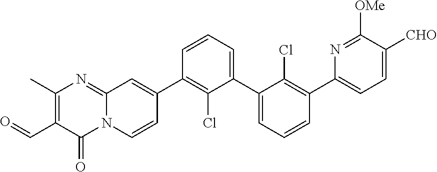 [SMILES: COc1nc(-c2cccc(-c3cccc(-c4ccn5c(=O)c(C=O)c(C)nc5c4)c3Cl)c2Cl)ccc1C=O]

To a solution of 8-(3-bromo-2-chlorophenyl)-2-methyl-4-oxo-4H-pyrido[1,2-a]pyrimidine-3-carbaldehyde (0.7 g, 1.85 mmol) in dioxane/H₂O (3:1, 8 mL) was added 6-(2-chloro-3-(4,4,5,5-tetramethyl-1,3,2-dioxaborolan-2-yl)phenyl)-2- (0.73 g, 1.95 mmol), [1,1′-Bis(di-tert-butylphosphino)ferrocene]dichloropalladium(II) (0.12 g, 0.18 mmol) and potassium phosphate (1.18 g, 5.55 mmol). The reaction was stirred at 110° C. for 0.5 hr, then it was filtered and concentrated under reduced pressure. The residue was purified by normal phase SiO₂ chromatography (0-100% EtOAc/petroleum ether) to afford 8-(2,2′-dichloro-3′-(5-formyl-6-methoxypyridin-2-yl)-[1,1′-biphenyl]-3-yl)-2-methyl-4-oxo-4H-pyrido[1,2-a]pyrimidine-3-carbaldehyde as a black solid (0.45 g, 44% yield, MS: 544 [M+H]⁺ (LCMS method J)).

### 8-(2,2′-Dichloro-3′-(6-methoxy-5-(((((S)-5-oxopyrrolidin-2-yl)methyl)amino) methyl) pyridin-2-yl)-[1,1′-biphenyl]-3-yl)-3-((((1r,3r)-3-hydroxy-3-methylcyclobutyl) amino)methyl)-2-methyl-4H-pyrido[1,2-a]pyrimidin-4-one

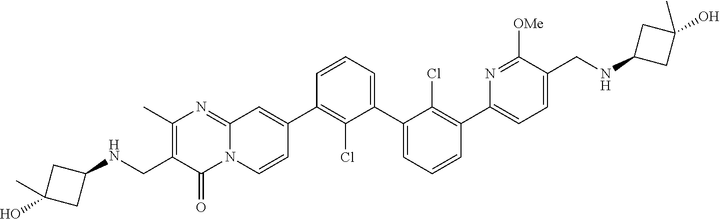 [SMILES: COc1nc(-c2cccc(-c3cccc(-c4ccn5c(=O)c(CN[C@H]6C[C@](C)(O)C6)c(C)nc5c4)c3Cl)c2Cl)ccc1CN[C@H]1C[C@](C)(O)C1]

To a solution of 8-(2,2′-dichloro-3′-(5-formyl-6-methoxypyridin-2-yl)-[1,1′-biphenyl]-3-yl)-2-methyl-4-oxo-4H-pyrido[1,2-a]pyrimidine-3-(0.09 g, 0.17 mmol) in DCM/MeOH (1:1, 4 mL) was added (1r,3r)-3-amino-1-methylcyclobutanol HCl (0.08 g, 0.58 mmol), sodium acetate (0.05 g, 0.66 mmol). The reaction was then stirred at r.t. for 1 hr. Sodium cyanoborohydride (0.04 g, 0.66 mmol) was added, and the reaction was stirred at r.t. for 2 hr. The reaction mixture was filtered and concentrated under reduced pressure. The residue was purified by reverse phase HPLC to afford 8-(2,2′-dichloro-3′-(5-((((1r,3r)-3-hydroxy-3-methylcyclobutyl)amino)methyl)-6-methoxypyridin-2-yl)-[1,1′-biphenyl]-3-yl)-3-((((1r,3r)-3-hydroxy-3-methylcyclobutyl)amino) methyl)-2-methyl-4H-pyrido[1,2-a]pyrimidin-4-one as a white solid (14.1 mg, 12% yield, MS: 714 [M+H]⁺ observed MS method A). ¹H NMR (400 MHz, Methanol-d₄): δ 9.06 (d, J=7.6 Hz, 1H), 7.72-7.70 (m, 2H), 7.66 (dd, J=1.2 Hz, J=7.6 Hz, 1H), 7.65-7.58 (m, 2H), 7.54-7.48 (m, 3H), 7.41 (dd, J=1.2 Hz, J=7.6 Hz, 1H), 7.24 (d, J=7.2 Hz, 1H), 4.04 (m, 3H), 3.83 (m, 2H), 3.73 (m, 2H), 3.59-3.46 (m, 2H), 2.61 (s, 3H), 2.35-2.28 (m, 4H), 1.91-1.86 (m, 4H), 1.36-1.35 (m, 6H).

### Example 230: 8-(2,2′-Dichloro-3′-(5-((((1s,3s)-3-hydroxy-3-methylcyclobutyl) amino)methyl)-6-methoxypyridin-2-yl)-[1,1′-biphenyl]-3-yl)-3-((((1s,3s)-3-hydroxy-3-methylcyclobutyl)amino)methyl)-2-methyl-4H-pyrido[1,2-a]pyrimidin-4-one

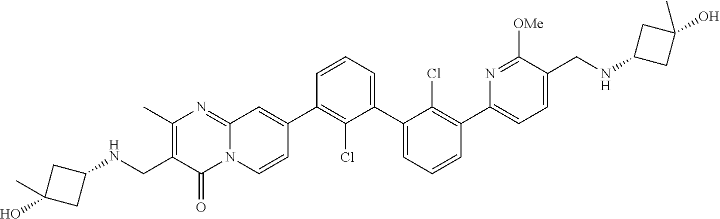 [SMILES: COc1nc(-c2cccc(-c3cccc(-c4ccn5c(=O)c(CN[C@H]6C[C@@](C)(O)C6)c(C)nc5c4)c3Cl)c2Cl)ccc1CN[C@H]1C[C@@](C)(O)C1]

This compound was prepared in a similar fashion to Example 205, using (1s,3s)-3-amino-1-methylcyclobutanol and tert-butyl (S)-((6-(2,2′-dichloro-3′-(3-formyl-2-methyl-4-oxo-4H-pyrido[1,2-a]pyrimidin-8-yl)-[1,1′-biphenyl]-3-yl)-2-methoxypyridin-3-yl)methyl)((5-oxopyrrolidin-2-yl)methyl)carbamate. MS: m/z found 714 [M+H]⁺, (Method A). ¹H NMR (400 MHz, Methanol-d₄): δ 9.06 (d, J=7.6 Hz, 1H), 7.72-7.61 (m, 5H), 7.53-7.51 (m, 3H), 7.41 (dd, J=7.6 Hz, J=1.6 Hz, 1H), 7.24 (d, J=7.2 Hz, 1H), 4.04 (s, 3H), 3.84 (s, 2H), 3.74 (s, 2H), 3.05-2.91 (m, 2H), 2.61 (m, 3H), 2.39-2.32 (m, 4H), 1.94-1.85 (m, 4H), 1.32-1.31 (m, 6H).

### Example 181: 8-(2,2′-Dichloro-3′-(5-((((S)-2-hydroxypropyl)amino)methyl)-6-methoxypyridin-2-yl)-[1,1′-biphenyl]-3-yl)-3-((((S)-2-hydroxypropyl)amino)methyl)-2-methyl-4H-pyrido[1,2-a]pyrimidin-4-one

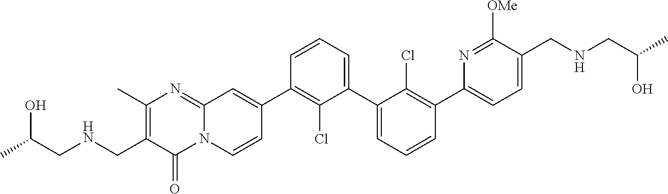 [SMILES: COc1nc(-c2cccc(-c3cccc(-c4ccn5c(=O)c(CNC[C@H](C)O)c(C)nc5c4)c3Cl)c2Cl)ccc1CNC[C@H](C)O]

This compound was prepared in a similar fashion to Example 205, using (S)-1-aminopropan-2-ol and tert-butyl (S)-((6-(2,2′-dichloro-3′-(3-formyl-2-methyl-4-oxo-4H-pyrido[1,2-a]pyrimidin-8-yl)-[1,1′-biphenyl]-3-yl)-2-methoxypyridin-3-yl)methyl)((5-oxopyrrolidin-2-yl)methyl) carbamate. MS: m/z found 662 [M+H]⁺, (Method A). ¹H NMR (400 MHz, Methanol-d₄): δ 9.07 (d, J=7.2 Hz, 1H), 8.52 (s, 1H), 7.80 (d, J=8 Hz, 1H), 7.74 (m, 1H), 7.66 (dd, J=1.6 Hz, J=7.6 Hz, 1H), 7.62-7.58 (m, 2H), 7.54-7.50 (m, 3H), 7.41 (dd, J=1.6 Hz, J=7.6 Hz, 1H), 7.30 (d, J=7.6 Hz, 1H), 4.29-4.21 (m, 2H), 4.09-4.00 (m, 7H), 3.06 (dd, J=1.6 Hz, J=12.4 Hz, 1H), 2.92-2.84 (m, 2H), 2.77-2.74 (m, 1H), 2.61 (s, 3H), 1.22 (dd, J=6.4 Hz, J=8 Hz, 6H).

### Example 229: 8-(2,2′-Dichloro-3′-(6-methoxy-5-(((((S)-5-oxopyrrolidin-2-yl)methyl)amino)methyl)pyridin-2-yl)-[1,1′-biphenyl]-3-yl)-3-((((1s,3s)-3-hydroxy-3-methylcyclobutyl)amino)methyl)-2-methyl-4H-pyrido[1,2-a]pyrimidin-4-one

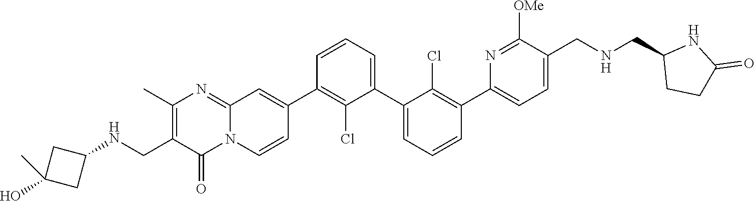 [SMILES: COc1nc(-c2cccc(-c3cccc(-c4ccn5c(=O)c(CN[C@H]6C[C@@](C)(O)C6)c(C)nc5c4)c3Cl)c2Cl)ccc1CNC[C@@H]1CCC(=O)N1]

This compound was prepared in a similar fashion to Example 193, using (1s,3s)-3-amino-1-methylcyclobutan-1-ol and tert-butyl (S)-((6-(2,2′-dichloro-3′-(3-formyl-2-methyl-4-oxo-4H-pyrido[1,2-a]pyrimidin-8-yl)-[1,1′-biphenyl]-3-yl)-2-methoxypyridin-3-yl)methyl)((5-oxopyrrolidin-2-yl)methyl)carbamate. MS: m/z found 727 [M+H]⁺ (Method A). ¹H NMR (400 MHz, Methanol-d₄): δ 9.09 (d, J=7.6 Hz, 1H), 7.80 (d, J=7.6 Hz, 1H), 7.76 (s, 1H), 7.66 (d, J=6.8 Hz, 1H), 7.62-7.50 (m, 5H), 7.41 (d, J=7.6 Hz, 1H), 7.29 (d, J=7.2 Hz, 1H), 4.21 (s, 2H), 4.08-4.05 (m, 5H), 3.95-3.92 (m, 1H), 3.58-3.54 (m, 1H), 2.95-2.91 (m, 2H), 2.61 (s, 3H), 2.55-2.50 (m, 2H), 2.35-2.30 (m, 5H), 1.86-1.84 (m, 1H), 1.39 (s, 3H).

### Example 269: (S)-Methyl 2-(((8-(2,2′-dichloro-3′-(6-methoxy-5-((((5-oxopyrrolidin-2-yl)methyl)amino)methyl)pyridin-2-yl)-[1,1′-biphenyl]-3-yl)-2-methyl-4-oxo-4H-pyrido[1,2-a]pyrimidin-3-yl)methyl)amino)acetate

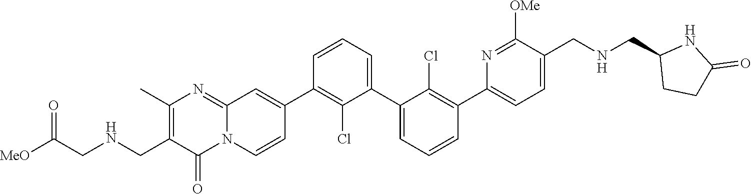 [SMILES: COC(=O)CNCc1c(C)nc2cc(-c3cccc(-c4cccc(-c5ccc(CNC[C@@H]6CCC(=O)N6)c(OC)n5)c4Cl)c3Cl)ccn2c1=O]

This compound was prepared in a similar fashion to Example 193, using methyl 2-aminoacetate and tert-butyl (S)-tert-butyl ((6-(2,2′-dichloro-3′-(3-formyl-2-methyl-4-oxo-4H-pyrido[1,2-a]pyrimidin-8-yl)-[1,1′-biphenyl]-3-yl)-2-methoxypyridin-3-yl)methyl)((5-oxopyrrolidin-2-yl)methyl)carbamate. MS: m/z found 727 [M+H]⁺, (Method AD). ¹H NMR (400 MHz, Methanol-d₄): δ 9.06 (d, J=7.2 Hz, 1H), 7.75 (d, J=7.2 Hz, 1H), 7.71-7.61 (m, 4H), 7.53-7.49 (m, 3H), 7.40 (dd, J=7.6 Hz, J=2 Hz, 1H), 7.26 (d, J=7.2 Hz, 1H), 4.04 (s, 3H), 3.95 (s, 2H), 3.90-3.81 (m, 3H), 3.72 (s, 3H), 3.51 (s, 2H), 2.76-2.67 (m, 2H), 2.62 (s, 3H), 2.38-2.29 (m, 3H), 1.86-1.79 (m, 1H).

### Example 265: (S)-Methyl 1-((8-(2,2′-dichloro-3′-(6-methoxy-5-(((((S)-5-oxopyrrolidin-2-yl)methyl)amino)methyl)pyridin-2-yl)-[1,1′-biphenyl]-3-yl)-2-methyl-4-oxo-4H-pyrido[1,2-a]pyrimidin-3-yl)methyl)pyrrolidine-3-carboxylate

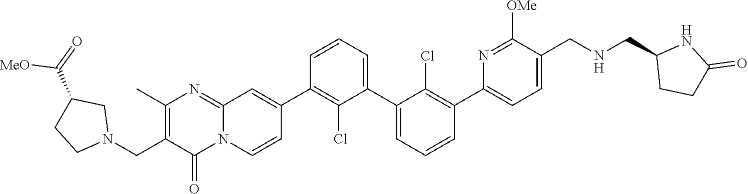 [SMILES: COC(=O)[C@H]1CCN(Cc2c(C)nc3cc(-c4cccc(-c5cccc(-c6ccc(CNC[C@@H]7CCC(=O)N7)c(OC)n6)c5Cl)c4Cl)ccn3c2=O)C1]

This compound was prepared in a similar fashion to Example 193, using (S)-methyl pyrrolidine-3-carboxylate HCl and tert-butyl (S)-tert-butyl ((6-(2,2′-dichloro-3′-(3-formyl-2-methyl-4-oxo-4H-pyrido[1,2-a]pyrimidin-8-yl)-[1,1′-biphenyl]-3-yl)-2-methoxypyridin-3-yl)methyl)((5-oxopyrrolidin-2-yl)methyl)carbamate. MS: m/z found 755 [M+H]⁺, (Method AD). ¹H NMR (400 MHz, Methanol-d₄): δ 9.13 (d, J=7.2 Hz, 1H), 7.92 (d, J=7.6 Hz, 1H), 7.81 (d, J=1.6 Hz, 1H), 7.70 (dd, J=7.6 Hz, J=1.6 Hz, 1H), 7.66-7.54 (m, 5H), 7.46 (dd, J=7.6 Hz, J=2 Hz, 1H), 7.39 (d, J=7.6 Hz, 1H), 4.41-4.33 (m, 2H), 4.12-3.44 (m, 13H), 3.30-3.26 (m, 2H), 2.68-2.38 (m, 9H), 1.98-1.92 (m, 1H).

### Example 285: 8-(2,2′-Dichloro-3′-(6-methoxy-5-(((((S)-5-oxopyrrolidin-2-yl)methyl) amino)methyl)pyridin-2-yl)-[1,1′-biphenyl]-3-yl)-3-((((3R,4S)-3-hydroxytetrahydro-2H-pyran-4-yl)amino)methyl)-2-methyl-4H-pyrido[1,2-a]pyrimidin-4-one

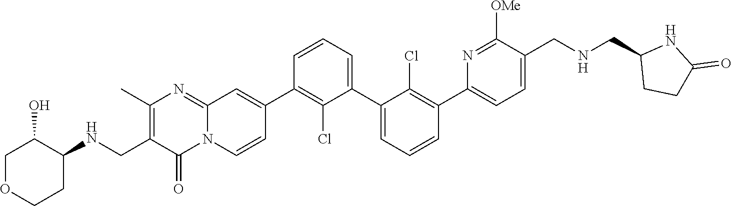 [SMILES: COc1nc(-c2cccc(-c3cccc(-c4ccn5c(=O)c(CN[C@H]6CCOC[C@@H]6O)c(C)nc5c4)c3Cl)c2Cl)ccc1CNC[C@@H]1CCC(=O)N1]

This compound was prepared in a similar fashion to Example 193, using (3R,4S)-4-aminotetrahydro-2H-pyran-3-ol and tert-butyl (S)-tert-butyl ((6-(2,2′-dichloro-3′-(3-formyl-2-methyl-4-oxo-4H-pyrido[1,2-a]pyrimidin-8-yl)-[1,1′-biphenyl]-3-yl)-2-methoxypyridin-3-yl)methyl)((5-oxopyrrolidin-2-yl)methyl)carbamate. MS: m/z found 743 [M+H]⁺, (Method K). ¹H NMR (400 MHz, Methanol-d₄): δ 9.04 (d, J=7.2 Hz, 1H), 7.72 (d, J=7.2 Hz, 1H), 7.69 (m, 1H), 7.65 (d, J=6.4 Hz, 1H), 7.61-7.56 (m, 2H), 7.51-7.45 (m, 3H), 7.38 (d, J=6.4 Hz, 1H), 7.23 (d, J=7.2 Hz, 1H), 4.01 (m, 4H), 3.97-3.91 (m, 2H), 3.87-3.78 (m, 4H), 3.42-3.40 (m, 2H), 3.08-3.03 (m, 1H), 2.73-2.57 (m, 6H), 2.35-2.15 (m, 4H), 1.85-1.76 (m, 1H), 1.55-1.46 (m, 1H).

### Example 287: (S)-8-(2,2′-Dichloro-3′-(6-methoxy-5-((((5-oxopyrrolidin-2-yl)methyl) amino)methyl)pyridin-2-yl)-[1,1′-biphenyl]-3-yl)-3-(((2-hydroxyethyl)amino)methyl)-2-methyl-4H-pyrido[1,2-a]pyrimidin-4-one

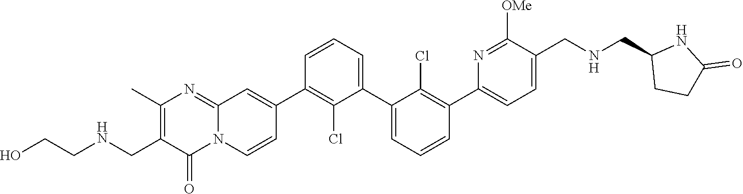 [SMILES: COc1nc(-c2cccc(-c3cccc(-c4ccn5c(=O)c(CNCCO)c(C)nc5c4)c3Cl)c2Cl)ccc1CNC[C@@H]1CCC(=O)N1]

This compound was prepared in a similar fashion to Example 193, using 2-aminoethanol and tert-butyl (S)-tert-butyl ((6-(2,2′-dichloro-3′-(3-formyl-2-methyl-4-oxo-4H-pyrido[1,2-a]pyrimidin-8-yl)-[1,1′-biphenyl]-3-yl)-2-methoxypyridin-3-yl)methyl)((5-oxopyrrolidin-2-yl)methyl)carbamate. MS: m/z found 687 [M+H]⁺, (Method AG). ¹H NMR (400 MHz, Methanol-d₄): δ 9.09 (d, J=7.6 Hz, 1H), 7.82 (d, J=7.6 Hz, 1H), 7.77 (d, J=1.6 Hz, 1H), 7.67-7.65 (m, 1H), 7.62-7.60 (m, 2H), 7.58-7.50 (m, 3H), 7.41 (dd, J=9.2 Hz, J=6.0 Hz, 1H), 7.31 (d, J=7.6 Hz, 1H), 4.56 (m, 1H), 4.39 (s, 2H), 4.11 (d, J=3.6 Hz, 2H), 4.05 (s, 3H), 3.96 (d, J=5.2 Hz, 1H), 3.88-3.85 (m, 2H), 3.27-3.25 (m, 2H), 3.01-2.98 (m, 2H), 2.62 (s, 3H), 2.39-2.32 (m, 2H), 1.91-1.86 (m, 1H).

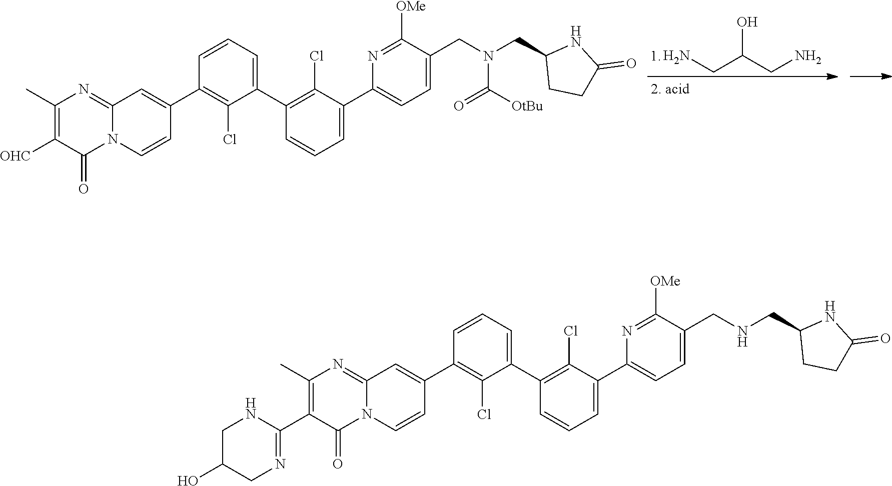 [SMILES: COc1nc(-c2cccc(-c3cccc(-c4ccn5c(=O)c(C=O)c(C)nc5c4)c3Cl)c2Cl)ccc1CN(C[C@@H]1CCC(=O)N1)C(=O)OC(C)(C)C ; NCC(O)CN ; COc1nc(-c2cccc(-c3cccc(-c4ccn5c(=O)c(C6=NCC(O)CN6)c(C)nc5c4)c3Cl)c2Cl)ccc1CNC[C@@H]1CCC(=O)N1]

### Example 290: (S)-8-(2,2′-Dichloro-3′-(6-methoxy-5-((((5-oxopyrrolidin-2-yl)methyl)amino)methyl)pyridin-2-yl)-[1,1′-biphenyl]-3-yl)-3-(((2-hydroxyethyl)amino)methyl)-2-methyl-4H-pyrido[1,2-a]pyrimidin-4-one

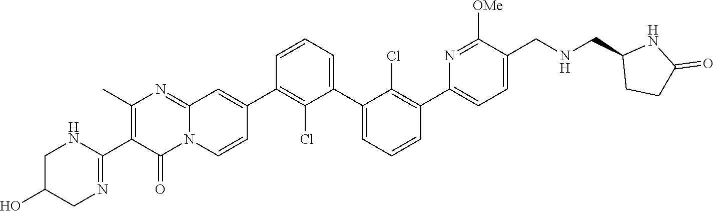 [SMILES: COc1nc(-c2cccc(-c3cccc(-c4ccn5c(=O)c(C6=NCC(O)CN6)c(C)nc5c4)c3Cl)c2Cl)ccc1CNC[C@@H]1CCC(=O)N1]

### tert-Butyl ((6-(2,2′-dichloro-3′-(3-(5-hydroxy-1,4,5,6-tetrahydropyrimidin-2-yl)-2-methyl-4-oxo-4H-pyrido[1,2-a]pyrimidin-8-yl)-[1,1′-biphenyl]-3-yl)-2-methoxypyridin-3-yl)methyl)(((S)-5-oxopyrrolidin-2-yl)methyl)carbamate

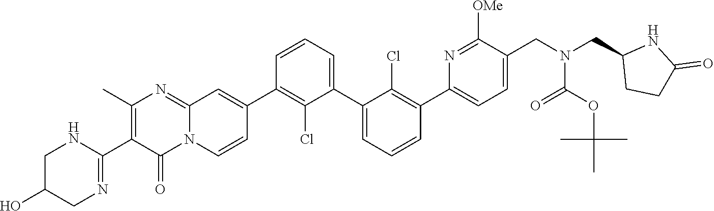 [SMILES: COc1nc(-c2cccc(-c3cccc(-c4ccn5c(=O)c(C6=NCC(O)CN6)c(C)nc5c4)c3Cl)c2Cl)ccc1CN(C[C@@H]1CCC(=O)N1)C(=O)OC(C)(C)C]

A mixture of (S)-tert-butyl ((6-(2,2′-dichloro-3′-(3-formyl-2-methyl-4-oxo-4H-pyrido[1,2-a]pyrimidin-8-yl)-[1,1′-biphenyl]-3-yl)-2-methoxypyridin-3-yl)methyl)((5-oxopyrrolidin-2-yl)methyl)carbamate (0.1 g, 0.13 mmol), 1,3-diaminopropan-2-ol (0.04 mg, 0.4 mmol), potassium carbonate (0.19 g, 1.35 mmol) and 12 (0.2 g, 0.8 mmol) in t-BuOH (5 mL) was degassed with N₂, and then stirred at 75° C. for 1 hr under N₂ atmosphere. The solution was basified with saturated aqueous sodium sulfite solution and then extracted with EtOAc (3×10 ml). The combined organic layers were washed with saturated aqueous brine solution (10 mL), dried over anhydrous sodium sulfate, filtered and concentrated under reduced pressure to give tert-butyl ((6-(2,2′-dichloro-3′-(3-(5-hydroxy-1,4,5,6-tetrahydropyrimidin-2-yl)-2-methyl-4-oxo-4H-pyrido[1,2-a]pyrimidin-8-yl)-[1,1′-biphenyl]-3-yl)-2-methoxypyridin-3-yl)methyl)(((S)-5-oxopyrrolidin-2-yl)methyl)carbamate (0.12 g) as yellow solid (m/z: 812 [M+H]⁺ observed (LCMS method C).

### 8-(2,2′-Dichloro-3′-(6-methoxy-5-(((((S)-5-oxopyrrolidin-2-yl)methyl)amino) methyl) pyridin-2-yl)-[1,1′-biphenyl]-3-yl)-3-(5-hydroxy-1,4,5,6-tetrahydropyrimidin-2-yl)-2-methyl-4H-pyrido[1,2-a]pyrimidin-4-one

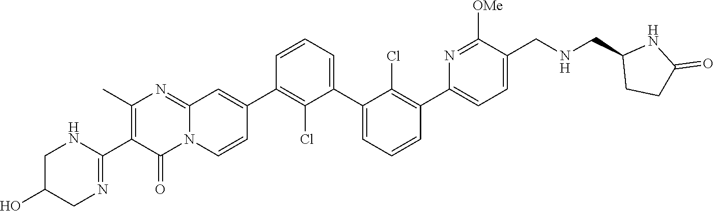 [SMILES: COc1nc(-c2cccc(-c3cccc(-c4ccn5c(=O)c(C6=NCC(O)CN6)c(C)nc5c4)c3Cl)c2Cl)ccc1CNC[C@@H]1CCC(=O)N1]

A mixture of tert-butyl ((6-(2,2′-dichloro-3′-(3-(5-hydroxy-1,4,5,6-tetrahydropyrimidin-2-yl)-2-methyl-4-oxo-4H-pyrido[1,2-a]pyrimidin-8-yl)-[1,1′-biphenyl]-3-yl)-2-methoxypyridin-3-yl)methyl)(((S)-5-oxopyrrolidin-2-yl)methyl)carbamate (0.12 g) and trifluoroacetic acid (1.5 mL) in DCM (4 mL) was degassed N₂, then the reaction was stirred at 25° C. for 1 hr under N₂ atmosphere. The reaction mixture was filtered and concentrated under reduced pressure. The residue was purified by reverse phase HPLC to afford 8-(2,2′-dichloro-3′-(6-methoxy-5-(((((S)-5-oxopyrrolidin-2-yl)methyl)amino)methyl)pyridin-2-yl)-[1,1′-biphenyl]-3-yl)-3-(5-hydroxy-1,4,5,6-tetrahydropyrimidin-2-yl)-2-methyl-4H-pyrido[1,2-a]pyrimidin-4-one as a yellow solid. (12.7 mg, 11% yield, m/z: 712 [M+H]⁺ observed, LCMS method K). ¹H NMR (400 MHz, DMSO-d₆): δ9.92 (s, 2H), 9.09 (d, J=7.6 Hz, 1H), 7.92 (d, J=1.6 Hz, 1H), 7.86 (d, J=7.6 Hz, 1H), 7.73-7.65 (m, 5H), 7.62-7.56 (m, 2H), 7.49-7.47 (m, 1H), 7.30 (d, J=7.6 Hz, 1H), 5.62 (d, J=2.4 Hz, 1H), 4.30 (s, 1H), 3.94 (s, 3H), 3.80-3.59 (m, 4H), 3.42 (s, 3H), 2.67 (m, 2H), 2.47 (m, 3H), 2.16-2.13 (m, 3H), 1.72 (m, 1H).

### Example 296: (S)-Methyl 1-((8-(2,2′-dichloro-3′-(6-methoxy-5-((((5-oxopyrrolidin-2-yl)methyl)amino)methyl)pyridin-2-yl)-[1,1′-biphenyl]-3-yl)-2-methyl-4-oxo-4H-pyrido[1,2-a]pyrimidin-3-yl)methyl)azetidine-3-carboxylate

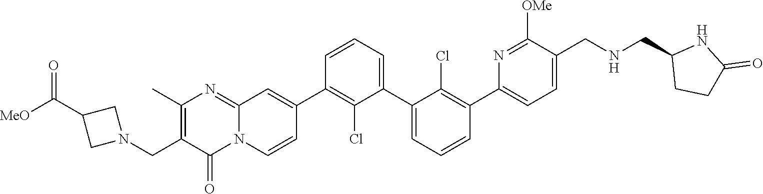 [SMILES: COC(=O)C1CN(Cc2c(C)nc3cc(-c4cccc(-c5cccc(-c6ccc(CNC[C@@H]7CCC(=O)N7)c(OC)n6)c5Cl)c4Cl)ccn3c2=O)C1]

This compound was prepared in a similar fashion to Example 193, using methyl azetidine-3-carboxylate and tert-butyl (S)-tert-butyl ((6-(2,2′-dichloro-3′-(3-formyl-2-methyl-4-oxo-4H-pyrido[1,2-a]pyrimidin-8-yl)-[1,1′-biphenyl]-3-yl)-2-methoxypyridin-3-yl)methyl)((5-oxopyrrolidin-2-yl)methyl)carbamate. MS: m/z found 741 [M+H]⁺, (Method A). ¹H NMR (400 MHz, Methanol-d₄): δ 9.11 (d, J=7.2 Hz, 1H), 7.92 (d, J=7.6 Hz, 1H), 7.79 (d, J=1.2 Hz, 1H), 7.71-7.69 (m, 1H), 7.65-7.55 (m, 5H), 7.47-7.45 (m, 1H), 7.38 (d, J=7.2 Hz, 1H), 4.61 (s, 2H), 4.51-4.31 (m, 6H), 4.12-4.05 (m, 4H), 3.82-3.74 (m, 4H), 3.31-3.22 (m, 2H), 2.66 (s, 3H), 2.48-2.36 (m, 3H), 1.99-1.90 (m, 1H).

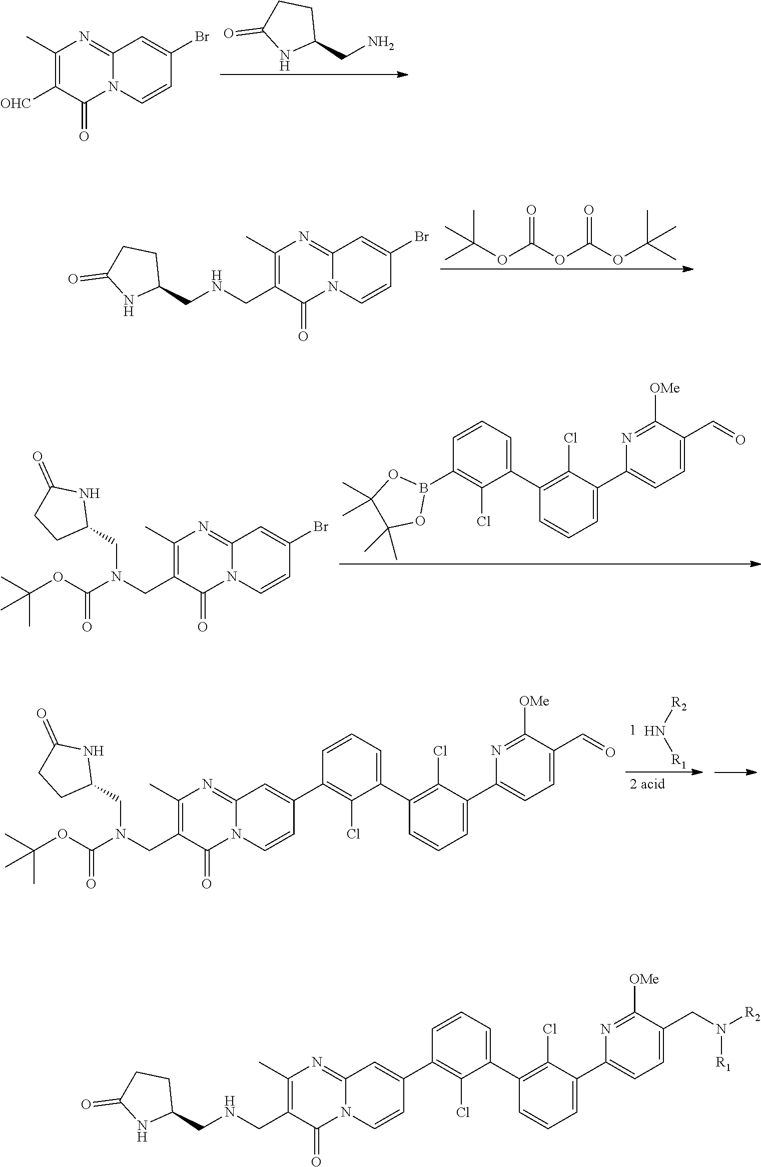

### Example 286: 8-(2,2′-Dichloro-3′-(5-((((3R,4S)-3-hydroxytetrahydro-2H-pyran-4-yl)amino)methyl)-6-methoxypyridin-2-yl)-[1,1′-biphenyl]-3-yl)-2-methyl-3-(((((S)-5-oxopyrrolidin-2-yl)methyl)amino)methyl)-4H-pyrido[1,2-a]pyrimidin-4-one

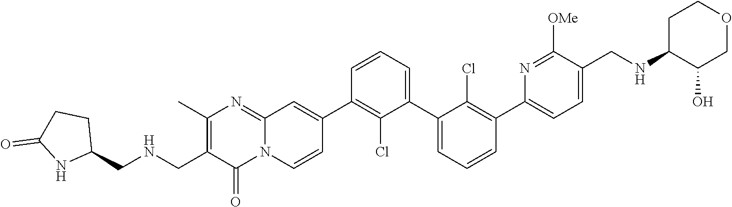 [SMILES: COc1nc(-c2cccc(-c3cccc(-c4ccn5c(=O)c(CNC[C@@H]6CCC(=O)N6)c(C)nc5c4)c3Cl)c2Cl)ccc1CN[C@H]1CCOC[C@@H]1O]

### (S)-8-Bromo-2-methyl-3-((((5-oxopyrrolidin-2-yl)methyl)amino)methyl)-4H-pyrido[1,2-a]pyrimidin-4-one

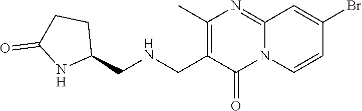 [SMILES: Cc1nc2cc(Br)ccn2c(=O)c1CNC[C@@H]1CCC(=O)N1]

A mixture of 8-bromo-2-methyl-4-oxo-4H-pyrido[1,2-a]pyrimidine-3-carbaldehyde (0.8 g, 3.0 mmol), (S)-5-(aminomethyl)pyrrolidin-2-one HCl (1.3 g, 8.9 mmol) and sodium acetate (0.74 g, 8.9 mmol) in DCM/MeOH (2:1, 45 mL) was degassed with N₂, and then the mixture was stirred at 20° C. for 1 hr under N₂ atmosphere. To this solution was added sodium cyanoborohydride (0.56 g, 8.9 mmol), and then the mixture was stirred at 20° C. for 0.5 hr under N₂ atmosphere, to give (S)-8-bromo-2-methyl-3-((((5-oxopyrrolidin-2-yl)methyl)amino)methyl)-4H-pyrido[1,2-a]pyrimidin-4-one as a yellow solution. m/z: 365 [M+H]⁺ observed (LCMS method J). The crude product was used directly without further purification.

### (S)-tert-Butyl ((8-bromo-2-methyl-4-oxo-4H-pyrido[1,2-a]pyrimidin-3-yl)methyl)((5-oxopyrrolidin-2-yl)methyl)carbamate

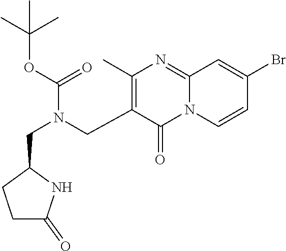 [SMILES: Cc1nc2cc(Br)ccn2c(=O)c1CN(C[C@@H]1CCC(=O)N1)C(=O)OC(C)(C)C]

To a solution of (S)-8-bromo-2-methyl-3-((((5-oxopyrrolidin-2-yl)methyl)amino)methyl)-4H-pyrido[1,2-a]pyrimidin-4-one in DCM/MeOH from previous step was added di-tert-butyl dicarbonate (1.4 mL) and triethylamine (1.25 mL). The mixture was degassed with N₂, and the reaction was stirred at 25° C. for 0.5 hr under N₂ atmosphere. Water was added (30 mL), and the reaction was then extracted with EtOAc (3×30 mL). The combined organic layers were washed with saturated aqueous brine solution (20 mL), dried over anhydrous sodium sulfate, filtered and concentrated under reduced pressure. The residue was purified by normal phase SiO₂ chromatography (0-100% EtOAc/petroleum ether) to afford (S)-tert-butyl ((8-bromo-2-methyl-4-oxo-4H-pyrido[1,2-a]pyrimidin-3-yl)methyl)((5-oxopyrrolidin-2-yl)methyl)carbamate as yellow solid (0.52 g, 34% yield). ¹H NMR (400 MHz, DMSO-d₆): δ 8.78 (d, J=7.6 Hz, 1H), 7.94 (d, J=1.6 Hz, 1H), 7.47 (dd, J=10 Hz, J=5.6 Hz, 1H), 6.91-6.86 (m, 1H), 3.77 (s, 1H), 3.53-3.49 (m, 2H), 3.20-3.18 (m, 2H), 2.95 (m, 2H), 2.43 (s, 3H), 1.69-1.62 (m, 2H), 1.39 (s, 9H).

### (S)-tert-Butyl ((8-(2,2′-dichloro-3′-(5-formyl-6-methoxypyridin-2-yl)-[1,1′-biphenyl]-3-yl)-2-methyl-4-oxo-4H-pyrido[1,2-a]pyrimidin-3-yl)methyl)((5-oxopyrrolidin-2-yl)methyl)carbamate

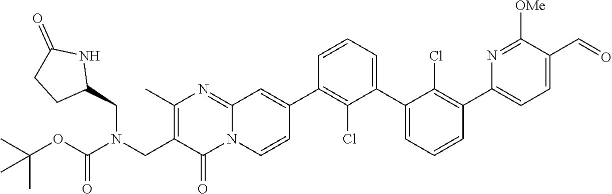

A mixture of (S)-tert-butyl ((8-bromo-2-methyl-4-oxo-4H-pyrido[1,2-a]pyrimidin-3-yl)methyl)((5-oxopyrrolidin-2-yl)methyl)carbamate (0.48 g, 1.0 mmol), 6-(2,2′-dichloro-3′-(4,4,5,5-tetramethyl-1,3,2-dioxaborolan-2-yl)-[1,1′-biphenyl]-3-yl)-2-methoxynicotinaldehyde (0.55 g, 1.1 mmol), potassium phosphate (0.66 g, 3.1 mmol) and Dichloro[1,1′-bis(di-1-propylphosphino)ferrocene]palladiurm (II) (0.07 g, 0.1 mmol) in dioxane/H₂O (5:1, 2.4 mL) was degassed N₂, and then the mixture was stirred at 80° C. for 2 hr under N₂. The reaction mixture was added to water (30 mL), and then extracted with EtOAc(3×30 mL). The combined organic layers were washed with saturated aqueous brine solution (20 mL), dried over anhydrous sodium sulfate, filtered and concentrated under reduced pressure. The residue was purified by normal phase SiO₂ chromatography (0-100% EtOAc/petroleum ether) to afford (S)-tert-butyl ((8-(2,2′-dichloro-3′-(5-formyl-6-methoxypyridin-2-yl)-[1,1′-biphenyl]-3-yl)-2-methyl-4-oxo-4H-pyrido[1,2-a]pyrimidin-3-yl)methyl)((5-oxopyrrolidin-2-yl)methyl)carbamate (0.4 g, 71% yield) as yellow oil. (m/z: 742 [M+H]⁺ observed, LCMS method AD).

### tert-Butyl ((8-(2,2′-dichloro-3′-(5-((((3R,4S)-3-hydroxytetrahydro-2H-pyran-4-yl)amino)methyl)-6-methoxypyridin-2-yl)-[1,1′-biphenyl]-3-yl)-2-methyl-4-oxo-4H-pyrido[1,2-a]pyrimidin-3-yl)methyl)(((S)-5-oxopyrrolidin-2-yl)methyl)carbamate

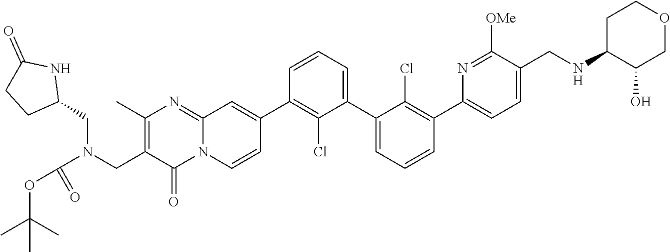 [SMILES: COc1nc(-c2cccc(-c3cccc(-c4ccn5c(=O)c(CN(C[C@@H]6CCC(=O)N6)C(=O)OC(C)(C)C)c(C)nc5c4)c3Cl)c2Cl)ccc1CN[C@H]1CCOC[C@@H]1O]

A mixture of (S)-tert-butyl ((8-(2,2′-dichloro-3′-(5-formyl-6-methoxypyridin-2-yl)-[1,1′-biphenyl]-3-yl)-2-methyl-4-oxo-4H-pyrido[1,2-a]pyrimidin-3-yl)methyl)((5-oxopyrrolidin-2-yl)methyl)carbamate (0.25 g, 0.34 mmol), (3R,4S)-4-aminotetrahydro-2H-pyran-3-ol (0.12 g, 1.0 mmol) and sodium acetate (0.08 g, 1.0 mmol) in DCM/MeOH (2:1, 15 mL) was degassed with N₂, then the mixture was stirred at 25° C. for 12 hr under N₂ atmosphere. Sodium cyanoborohydride (0.08 g, 1.3 mmol) was added, then the mixture was stirred at 25° C. for 0.5 hr under N₂ atmosphere, yielding tert-butyl N-[[8-[2-chloro-3-[2-chloro-3-[5-[[[(3R,4S)-3-hydroxytetrahydropyran-4-yl]amino]methyl]-6-methoxy-2-pyridyl]phenyl]phenyl]-2-methyl-4-oxo-pyrido[1,2-a]pyrimidin-3-yl]methyl]-N-[[(2S)-5-oxopyrrolidin-2-yl]methyl]carbamate as a yellow solution in DCM/MeOH (2:1, 15 mL) (m/z: 843 [M+H]⁺ observed, LCMS method AD). The crude product was used directly without further purification.

### 8-(2,2′-Dichloro-3′-(5-((((3R,4S)-3-hydroxytetrahydro-2H-pyran-4-yl)amino)methyl)-6-methoxypyridin-2-yl)-[1,1′-biphenyl]-3-yl)-2-methyl-3-(((((S)-5-oxopyrrolidin-2-yl)methyl)amino)methyl)-4H-pyrido[1,2-a]pyrimidin-4-one

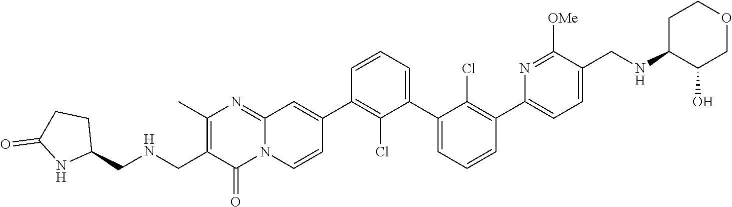 [SMILES: COc1nc(-c2cccc(-c3cccc(-c4ccn5c(=O)c(CNC[C@@H]6CCC(=O)N6)c(C)nc5c4)c3Cl)c2Cl)ccc1CN[C@H]1CCOC[C@@H]1O]

To a solution of tert-butyl N-[[8-[2-chloro-3-[2-chloro-3-[5-[[[(3R,4S)-3-hydroxytetrahydropyran-4-yl]amino]methyl]-6-methoxy-2-pyridyl]phenyl]phenyl]-2-methyl-4-oxo-pyrido[1,2-a]pyrimidin-3-yl]methyl]-N-[[(2S)-5-oxopyrrolidin-2-yl]methyl]carbamate in DCM/MeOH (2:1, 15 mL) was added trifluoroacetic acid (1.5 mL) in DCM (7.5 mL). The reaction degassed with N₂, then stirred at 25° C. for 1 hr under N₂ atmosphere. The reaction mixture was filtered and concentrated under reduced pressure. The residue was purified by reverse phase HPLC to afford 8-(2,2′-dichloro-3′-(5-((((3R,4S)-3-hydroxytetrahydro-2H-pyran-4-yl)amino)methyl)-6-methoxypyridin-2-yl)-[1,1′-biphenyl]-3-yl)-2-methyl-3-(((((S)-5-oxopyrrolidin-2-yl)methyl)amino)methyl)-4H-pyrido[1,2-a]pyrimidin-4-one formic acid salt as a white solid. (27.2 mg, 10% yield, m/z: 743 [M+H]⁺ observed, LCMS method K). ¹H NMR (400 MHz, Methanol-d₄): δ9.08 (d, J=7.6 Hz, 1H), 8.41 (br, 1H), 7.86 (d, J=7.6 Hz, 1H), 7.74 (d, J=1.2, 1H), 7.67 (dd, J=9.6 Hz, J=5.6 Hz, 1H), 7.63-7.58 (m, 2H), 7.55-7.50 (m, 3H), 7.42 (dd, J=9.2 Hz, J=6.0 Hz, 1H), 7.33 (d, J=7.2 Hz, 1H), 4.28-4.21 (m, 4H), 4.08 (s, 3H), 4.05-3.93 (m, 3H), 3.69-3.62 (m, 1H), 3.44-3.38 (m, 1H), 3.12-3.02 (m, 4H), 2.62 (s, 3H), 2.38-2.33 (m, 3H), 2.22-2.18 (m, 1H), 1.88 (d, J=7.2 Hz, 1H), 1.79-1.70 (m, 1H).

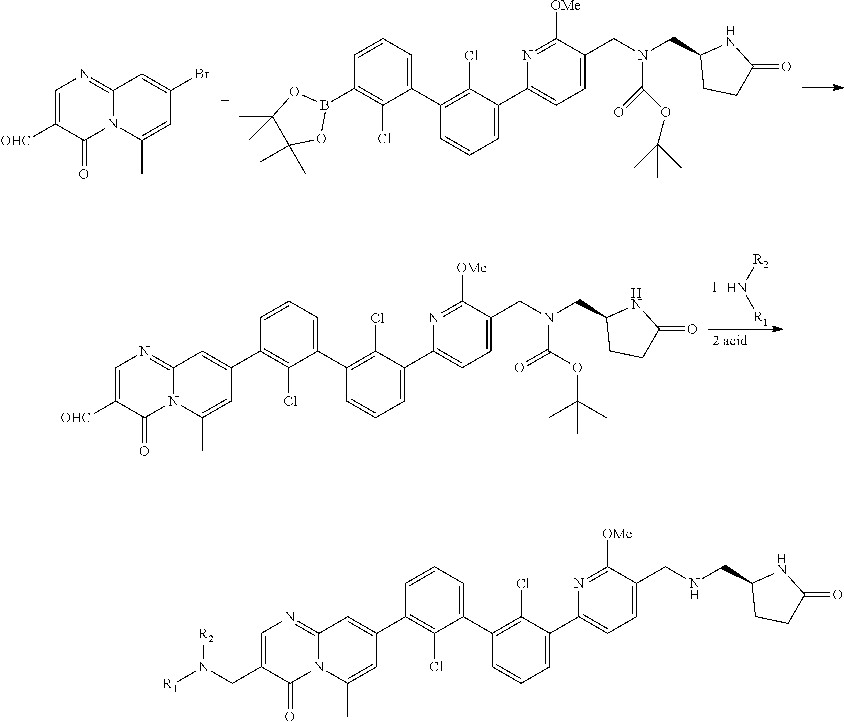

### Example 226: 8-(2,2′-Dichloro-3′-(6-methoxy-5-(((((S)-5-oxopyrrolidin-2-yl)methyl) amino)methyl)pyridin-2-yl)-[1,1′-biphenyl]-3-yl)-3-((((S)-2-hydroxypropyl)amino)methyl)-6-methyl-4H-pyrido[1,2-a]pyrimidin-4-one

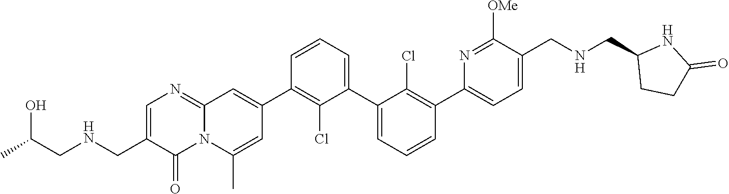 [SMILES: COc1nc(-c2cccc(-c3cccc(-c4cc(C)n5c(=O)c(CNC[C@H](C)O)cnc5c4)c3Cl)c2Cl)ccc1CNC[C@@H]1CCC(=O)N1]

### (S)-tert-Butyl ((6-(2,2′-dichloro-3′-(3-formyl-6-methyl-4-oxo-4H-pyrido[1,2-a]pyrimidin-8-yl)-[1,1′-biphenyl]-3-yl)-2-methoxypyridin-3-yl)methyl)((5-oxopyrrolidin-2-yl)methyl) carbamate

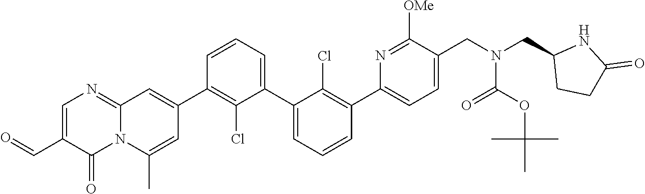 [SMILES: COc1nc(-c2cccc(-c3cccc(-c4cc(C)n5c(=O)c(C=O)cnc5c4)c3Cl)c2Cl)ccc1CN(C[C@@H]1CCC(=O)N1)C(=O)OC(C)(C)C]

To a mixture of 8-bromo-6-methyl-4-oxo-4H-pyrido[1,2-a]pyrimidine-3-carbaldehyde (0.15 g, 0.56 mmol) and (S)-tert-butyl ((6-(2,2′-dichloro-3′-(4,4,5,5-tetramethyl-1,3,2-dioxaborolan-2-yl)-[1,1′-biphenyl]-3-yl)-2-methoxypyridin-3-yl)methyl)((5-oxopyrrolidin-2-yl)methyl)carbamate (0.41 g, 0.6 mmol) in 1,4-dioxane/H₂O mixture (5:1, 6 mL) was added 1,1′-bis(diphenylphosphino)ferrocene-palladium(ii) dichloride dichloromethane complex (0.02 g, 0.03 mmol) and potassium carbonate (0.23 g, 1.68 mmol) in one portion under N₂ atmosphere. The mixture was stirred at 110° C. for 0.5 h. The mixture was concentrated, and to the residue was added water (25 mL) and saturated aqueous brine solution (20 mL). The mixture was extracted with ethyl acetate/THF mixture (1:1, 50 mL). The organic phase was dried with anhydrous sodium sulfate, filtered and concentrated. The residue was purified by normal phase SiO₂ chromatography (0-65% 50% THF in ethyl acetate/petroleum ether) to afford (S)-tert-butyl ((6-(2,2′-dichloro-3′-(3-formyl-6-methyl-4-oxo-4H-pyrido[1,2-a]pyrimidin-8-yl)-[1,1′-biphenyl]-3-yl)-2-methoxypyridin-3-yl)methyl)((5-oxopyrrolidin-2-yl)methyl)carbamate (110 mg, 16% yield) as a yellow solid. ¹H NMR (400 MHz, DMSO-d₆): δ10.05 (s, 1H), 8.58 (s, 1H), 7.73 (m, 2H), 7.65-7.60 (m, 3H), 7.57-7.53 (m, 2H), 7.46-7.43 (m, 3H), 7.26 (m, 1H), 4.45-4.32 (m, 2H), 3.92 (s, 3H), 3.74 (m, 1H), 3.08 (s, 3H), 2.07-1.89 (m, 5H), 1.34 (m, 9H).

### tert-Butyl ((6-(2,2′-dichloro-3′-(3-((((S)-2-hydroxypropyl)amino)methyl)-6-methyl-4-oxo-4H-pyrido[1,2-a]pyrimidin-8-yl)-[1,1′-biphenyl]-3-yl)-2-methoxypyridin-3-yl)methyl)(((S)-5-oxopyrrolidin-2-yl)methyl)carbamate

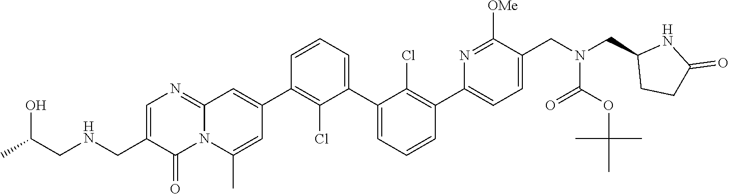 [SMILES: COc1nc(-c2cccc(-c3cccc(-c4cc(C)n5c(=O)c(CNC[C@H](C)O)cnc5c4)c3Cl)c2Cl)ccc1CN(C[C@@H]1CCC(=O)N1)C(=O)OC(C)(C)C]

To a mixture of (S)-tert-butyl ((6-(2,2′-dichloro-3′-(3-formyl-6-methyl-4-oxo-4H-pyrido[1,2-a]pyrimidin-8-yl)-[1,1′-biphenyl]-3-yl)-2-methoxypyridin-3-yl)methyl)((5-oxopyrrolidin-2-yl)methyl)carbamate (100 mg, 0.13 mmol) and (S)-1-aminopropan-2-ol (20.2 mg, 0.27 mmol) in DCM (3 mL) was added sodium acetate (22 mg, 0.27 mmol) and sodium triacetoxyborohydride (85.6 mg, 0.4 mmol) in one portion. The mixture was stirred at r.t. for 12 h under N₂ atmosphere. The product obtained, tert-butyl ((6-(2,2′-dichloro-3′-(3-((((S)-2-hydroxypropyl)amino)methyl)-6-methyl-4-oxo-4H-pyrido[1,2-a]pyrimidin-8-yl)-[1,1′-biphenyl]-3-yl)-2-methoxypyridin-3-yl)methyl)(((S)-5-oxopyrrolidin-2-yl)methyl)carbamate (as a yellow solution) was used in the next step without work-up or purification (m/z found 801 [M+H]⁺ Method AF).

### 8-(2,2′-Dichloro-3′-(6-methoxy-5-(((((S)-5-oxopyrrolidin-2-yl)methyl)amino) methyl) pyridin-2-yl)-[1,1′-biphenyl]-3-yl)-3-((((S)-2-hydroxypropyl)amino)methyl)-6-methyl-4H-pyrido[1,2-a]pyrimidin-4-one

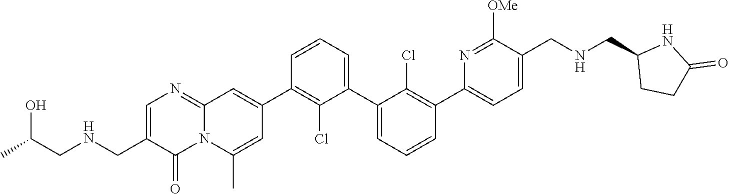 [SMILES: COc1nc(-c2cccc(-c3cccc(-c4cc(C)n5c(=O)c(CNC[C@H](C)O)cnc5c4)c3Cl)c2Cl)ccc1CNC[C@@H]1CCC(=O)N1]

To a solution of tert-butyl ((6-(2,2′-dichloro-3′-(3-((((S)-2-hydroxypropyl)amino)methyl)-6-methyl-4-oxo-4H-pyrido[1,2-a]pyrimidin-8-yl)-[1,1′-biphenyl]-3-yl)-2-methoxypyridin-3-yl)methyl)(((S)-5-oxopyrrolidin-2-yl)methyl)carbamate in DCM (3 mL) from the previous step was added trifluoroacetic acid (1.5 mL, 20 mmol). The mixture was stirred at r.t. for 1 h. The mixture was concentrated, and the residue was purified by reverse phase HPLC to afford 8-(2,2′-dichloro-3′-(6-methoxy-5-(((((S)-5-oxopyrrolidin-2-yl)methyl)amino) methyl)pyridin-2-yl)-[1,1′-biphenyl]-3-yl)-3-((((S)-2-hydroxypropyl)amino)methyl)-6-methyl-4H-pyrido[1,2-a]pyrimidin-4-one as a formic acid salt (19.2 mg, 20% yield for two steps, m/z 701 [M+H]⁺ observed, Method AH) as a yellow solid. ¹H NMR (400 MHz, DMSO-d₆): δ 8.36 (s, 1H), 7.93 (d, J=7.2 Hz, 1H), 7.71-7.56 (m, 6H), 7.50-7.48 (m, 1H), 7.39-7.37 (m, 1H), 7.24 (m, 1H), 5.32 (d, J=5.2 Hz, 1H), 4.12-4.05 (m, 4H), 3.98-3.48 (m, 5H), 3.07-3.00 (m, 6H), 2.80-2.75 (m, 1H), 2.22-2.13 (m, 3H), 1.78-1.76 (m, 1H), 1.12 (d, J=6.4 Hz, 3H). ¹H NMR (400 MHz, Methanol-d₄): δ 8.33 (s, 1H), 7.88 (d, J=7.6 Hz, 1H), 7.70-7.68 (m, 1H), 7.62-7.61 (m, 3H), 7.57-7.51 (m, 2H), 7.45-7.42 (m, 1H), 7.36 (d, J=7.2 Hz, 1H), 7.21 (s, 1H), 4.29-4.23 (m, 4H), 4.16-4.03 (m, 5H), 3.33-3.11 (m, 6H), 2.97-2.91 (m, 1H), 2.50-2.33 (m, 3H), 1.97-1.91 (m, 1H), 1.27 (d, J=6 Hz, 3H).

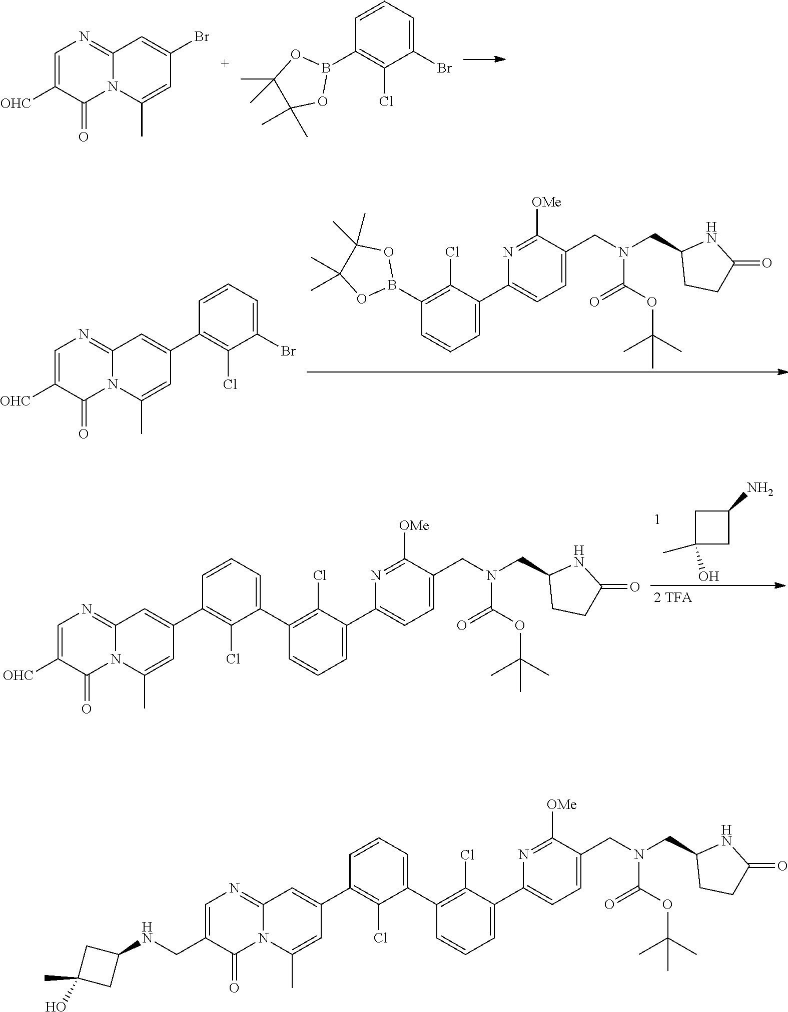

### Example 211: 8-(2,2′-Dichloro-3′-(6-methoxy-5-(((((S)-5-oxopyrrolidin-2-yl)methyl)amino)methyl)pyridin-2-yl)-[1,1′-biphenyll-3-yl)-3-((((1r,3r)-3-hydroxy-3-methylcyclobutyl)amino)methyl)-6-methyl-4H-pyrido[1,2-a]pyrimidin-4-one

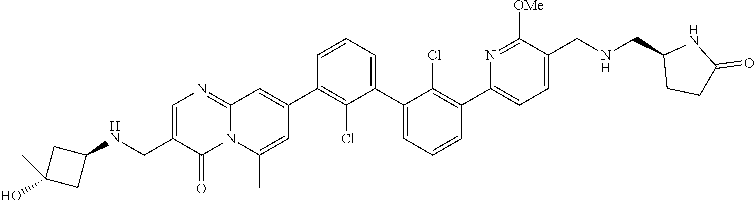 [SMILES: COc1nc(-c2cccc(-c3cccc(-c4cc(C)n5c(=O)c(CN[C@H]6C[C@](C)(O)C6)cnc5c4)c3Cl)c2Cl)ccc1CNC[C@@H]1CCC(=O)N1]

### 8-(3-Bromo-2-chlorophenyl)-6-methyl-4-oxo-4H-pyrido[1,2-a]pyrimidine-3-carbaldehyde

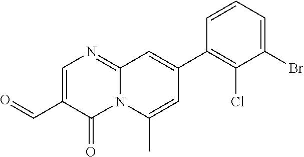 [SMILES: Cc1cc(-c2cccc(Br)c2Cl)cc2ncc(C=O)c(=O)n12]

To a mixture of 8-bromno-6-methyl-4-oxo-4H-pyrido[1,2-a]pyrimidine-3-carbaldehyde (1.03 g, 3.86 mmol) and 2-(3-bromo-2-chlorophenyl)-4,4,5,5-tetramethyl-1,3,2-dioxaborolane (1.35 g, 4.24 mmol) in 1,4-dioxane/H₂O mixture (5:1, 48 mL) was added 1,1′-bis(diphenylphosphino) ferrocene-palladium(ii) dichloride dichloromethane complex (0.16 g, 0.19 mmol) and potassium carbonate (1.6 g, 11.6 mmol) in one portion. The mixture was stirred at 100° C. for 0.5 h under N₂ atmosphere. The mixture was concentrated, and to the residue was added water (20 mL), petroleum ether (10 mL), MeCN (20 mL) and ethyl acetate (10 mL). The resulting yellow solid was purified by normal phase SiO₂ chromatography (0-30% 50% THE in ethyl acetate/petroleum ether) to afford 8-(3-bromo-2-chlorophenyl)-6-methyl-4-oxo-4H-pyrido[1,2-a]pyrimidine-3-carbaldehyde (0.75 g, 37% yield) as a yellow solid. ¹H NMR (400 MHz, DMSO-d₆): δ 10.08 (s, 1H), 8.60 (s, 1H), 7.97 (d, J=8 Hz, 1H), 7.74 (s, 1H), 7.60 (d, J=7.6 Hz, 1H), 7.50-7.48 (m, 2H), 3.09 (s, 3H).

### (S)-tert-Butyl ((6-(2,2′-dichloro-3′-(3-formyl-6-methyl-4-oxo-4H-pyrido[1,2-a]pyrimidin-8-yl)-[1,1′-biphenyl]-3-yl)-2-methoxypyridin-3-yl)methyl)((5-oxopyrrolidin-2-yl)methyl) carbamate

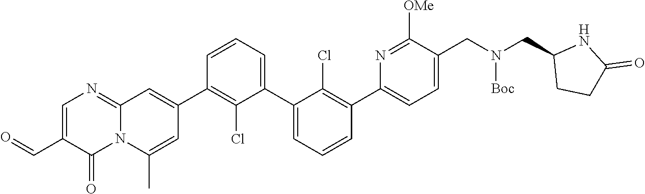 [SMILES: COc1nc(-c2cccc(-c3cccc(-c4cc(C)n5c(=O)c(C=O)cnc5c4)c3Cl)c2Cl)ccc1CN(C[C@@H]1CCC(=O)N1)C(=O)OC(C)(C)C]

To a mixture of 8-(3-bromo-2-chlorophenyl)-6-methyl-4-oxo-4H-pyrido[1,2-a]pyrimidine-3-carbaldehyde (0.55 g, 1.46 mmol) and (S)-tert-butyl ((6-(2-chloro-3-(4,4,5,5-tetramethyl-1,3,2-dioxaborolan-2-yl)phenyl)-2-methoxypyridin-3-yl)methyl)((5-oxopyrrolidin-2-yl)methyl)carbamate (1.13 g, 1.38 mmol) in 1,4-dioxane/H₂O mixture (5:1, 4.8 mL) was added [1,1′-Bis(di-tert-butylphosphino)ferrocene] dichloropalladium (II) (0.09 g, 0.15 mmol) and potassium phosphate (0.93 g, 4.37 mmol) in one portion. The mixture was stirred at 110° C. for 0.5 h under N₂ atmosphere. The mixture was concentrated, and to the residue was added water (50 mL) and saturated aqueous brine solution (50 mL). The mixture was extracted with ethyl acetate/THF mixture (1:1, 200 mL). The organic phase was dried with anhydrous sodium sulfate, filtered and concentrated. The residue was purified by normal phase SiO₂ chromatography (20-80% 50% THE in ethyl acetate/petroleum ether) to afford 0.33 g of the product. 0.215 g of the product was further purified by reverse phase HPLC to afford (S)-tert-butyl ((6-(2,2′-dichloro-3′-(3-formyl-6-methyl-4-oxo-4H-pyrido[1,2-a]pyrimidin-8-yl)-[1,1′-biphenyl]-3-yl)-2-methoxypyridin-3-yl)methyl)((5-oxopyrrolidin-2-yl)methyl)carbamate tert-butyl (40 mg, m/z: 742 [M+H]⁺ observed, Method AI) as a yellow solid.

### tert-butyl ((6-(2,2′-Dichloro-3′-(3-((((1r,3r)-3-hydroxy-3-methylcyclobutyl)amino)methyl)-6-methyl-4-oxo-4H-pyrido[1,2-a]pyrimidin-8-yl)-[1,1′-biphenyl]-3-yl)-2-methoxypyridin-3-yl)methyl)(((S)-5-oxopyrrolidin-2-yl)methyl)carbamate

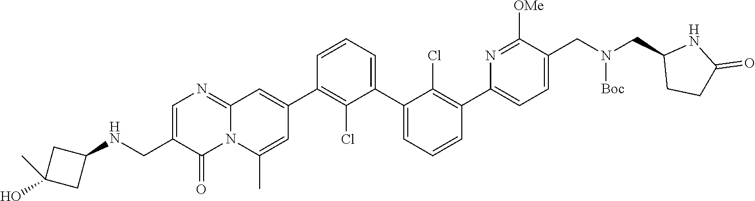 [SMILES: COc1nc(-c2cccc(-c3cccc(-c4cc(C)n5c(=O)c(CN[C@H]6C[C@](C)(O)C6)cnc5c4)c3Cl)c2Cl)ccc1CN(C[C@@H]1CCC(=O)N1)C(=O)OC(C)(C)C]

To a mixture of (S)-tert-butyl ((6-(2,2′-dichloro-3′-(3-formyl-6-methyl-4-oxo-4H-pyrido[1,2-a]pyrimidin-8-yl)-[1,1′-biphenyl]-3-yl)-2-methoxypyridin-3-yl)methyl)((5-oxopyrrolidin-2-yl)methyl)carbamate (35 mg, 0.05 mmol) and 3-amino-1-methyl-cyclobutanol as hydrochloride salt (19 mg, 0.14 mmol) in DCM (1.5 mL) was added sodium acetate (5.8 mg, 0.07 mmol) and sodium triacetoxyborohydride (30 mg, 0.14 mmol) in one portion at under N₂ atmosphere. The mixture was stirred at r.t. for 1.5 h. Tert-butyl ((6-(2,2′-dichloro-3′-(3-((((1r,3r)-3-hydroxy-3-methylcyclobutyl)amino)methyl)-6-methyl-4-oxo-4H-pyrido[1,2-a]pyrimidin-8-yl)-[1,1′-biphenyl]-3-yl)-2-methoxypyridin-3-yl)methyl)(((S)-5-oxopyrrolidin-2-yl)methyl)carbamate (m/z: 827 [M+H]⁺ observed, Method AF) was obtained in a DCM mixture (1.5 mL) as a yellow solution. The crude product was used for next step without purification.

### 8-(2,2′-Dichloro-3′-(6-methoxy-5-(((((S)-5-oxopyrrolidin-2-yl)methyl)amino)methyl) pyridin-2-yl)-[1,1′-biphenyl]-3-yl)-3-((((1r,3r)-3-hydroxy-3-methylcyclobutyl)amino) methyl)-6-methyl-4H-pyrido[1,2-a]pyrimidin-4-one

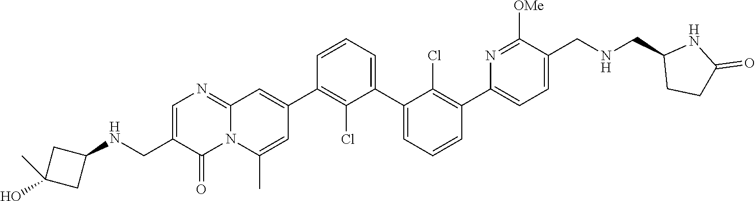 [SMILES: COc1nc(-c2cccc(-c3cccc(-c4cc(C)n5c(=O)c(CN[C@H]6C[C@](C)(O)C6)cnc5c4)c3Cl)c2Cl)ccc1CNC[C@@H]1CCC(=O)N1]

To a solution of tert-butyl ((6-(2,2′-dichloro-3′-(3-((((1r,3r)-3-hydroxy-3-methylcyclobutyl)amino)methyl)-6-methyl-4-oxo-4H-pyrido[1,2-a]pyrimidin-8-yl)-[1,1′-biphenyl]-3-yl)-2-methoxypyridin-3-yl)methyl)(((S)-5-oxopyrrolidin-2-yl)methyl)carbamate in DCM (1.5 mL) from the previous step was added trifluoroacetic acid (0.3 mL, 4.05 mmol). The mixture was stirred at r.t. for 1 h. The mixture was concentrated. The residue was purified by reverse phase HPLC to afford 8-(2,2′-dichloro-3′-(6-methoxy-5-(((((S)-5-oxopyrrolidin-2-yl)methyl)amino)methyl)pyridin-2-yl)-[1,1′-biphenyl]-3-yl)-3-((((1r,3r)-3-hydroxy-3-methylcyclobutyl)amino)methyl)-6-methyl-4H-pyrido[1,2-a]pyrimidin-4-one as a light yellow oil (9.8 mg, 28% yield for two steps, m/z: 727 [M+H]⁺ observed, (Method AE). ¹H NMR (400 MHz, Methanol-d₄): δ 8.31 (s, 1H), 7.83-7.81 (m, 1H), 7.68 (dd, J=8 Hz, J=2 Hz, 1H), 7.61-7.60 (m, 3H), 7.58-7.49 (m, 2H), 7.41 (dd, J=7.6 Hz, J=2 Hz, 1H), 7.30 (d, J=7.6 Hz, 1H), 7.20 (m, 1H), 4.07-3.94 (m, 9H), 3.18 (s, 3H), 2.93-2.92 (m, 2H), 2.45-2.28 (m, 7H), 1.92-1.83 (m, 1H), 1.42 (s, 3H).

### Example 254: 8-(2,2′-Dichloro-3′-(6-methoxy-5-(((((S)-5-oxopyrrolidin-2-yl)methyl)amino) methyl)pyridin-2-yl)-[1,1′-biphenyl]-3-yl)-3-((((1s,3s)-3-hydroxy-3-methylcyclobutyl) amino)methyl)-6-methyl-4H-pyrido[1,2-a]pyrimidin-4-one

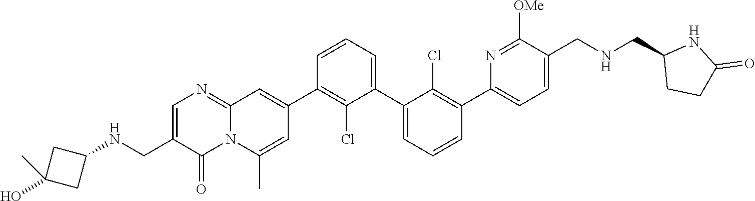 [SMILES: COc1nc(-c2cccc(-c3cccc(-c4cc(C)n5c(=O)c(CN[C@H]6C[C@@](C)(O)C6)cnc5c4)c3Cl)c2Cl)ccc1CNC[C@@H]1CCC(=O)N1]

This compound was prepared in a similar fashion to Example 226, using (1s,3s)-3-amino-1-methylcyclobutan-1-ol and tert-butyl (S)-((6-(2,2′-dichloro-3′-(3-formyl-6-methyl-4-oxo-4H-pyrido[1,2-a]pyrimidin-8-yl)-[1,1′-biphenyl]-3-yl)-2-methoxypyridin-3-yl)methyl)((5-oxopyrrolidin-2-yl)methyl)carbamate. MS: m/z found 727 [M+H]⁺, (Method AD). ¹H NMR (400 MHz, Methanol-d₄): δ 8.31 (s, 1H), 7.84 (d, J=7.6 Hz, 1H), 7.69 (dd, J=7.6 Hz, J=1.6 Hz, 1H), 7.62-7.61 (m, 3H), 7.56-7.50 (m, 2H), 7.42 (dd, J=7.6 Hz, J=1.6 Hz, 1H), 7.33 (d, J=7.6 Hz, 1H), 7.21 (m, 1H), 4.15-4.08 (m, 7H), 4.01-3.97 (m, 1H), 3.59-3.49 (m, 1H), 3.19 (s, 3H), 3.01-2.99 (m, 2H), 2.54-2.49 (m, 2H), 2.40-2.30 (m, 5H), 1.93-1.85 (m, 1H), 1.40 (s, 3H).

### Example 291: (S)-Methyl 1-((8-(2,2′-dichloro-3′-(6-methoxy-5-((((5-oxopyrrolidin-2-yl)methyl)amino)methyl)pyridin-2-yl)-[1,1′-biphenyl]-3-yl)-6-methyl-4-oxo-4H-pyrido[1,2-a]pyrimidin-3-yl)methyl)azetidine-3-carboxylate

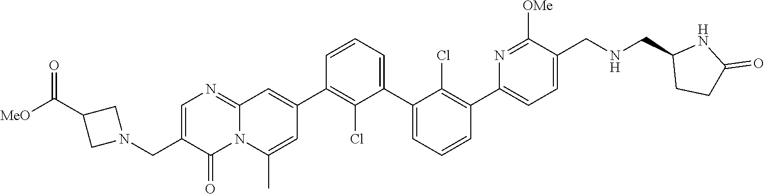 [SMILES: COC(=O)C1CN(Cc2cnc3cc(-c4cccc(-c5cccc(-c6ccc(CNC[C@@H]7CCC(=O)N7)c(OC)n6)c5Cl)c4Cl)cc(C)n3c2=O)C1]

This compound was prepared in a similar fashion to Example 226, using azetidine-3-carboxylate and tert-butyl (S)-((6-(2,2′-dichloro-3′-(3-formyl-6-methyl-4-oxo-4H-pyrido[1,2-a]pyrimidin-8-yl)-[1,1′-biphenyl]-3-yl)-2-methoxypyridin-3-yl)methyl)((5-oxopyrrolidin-2-yl)methyl)carbamate. MS: m/z found 741 [M+H]⁺, (Method A). ¹H NMR (400 MHz, Methanol-d₄): δ 8.23 (s, 1H), 7.80 (d, J=7.6 Hz, 1H), 7.68 (dd, J=8 Hz, J=1.6 Hz, 1H), 7.60 (d, J=4.4 Hz, 2H), 7.56-7.50 (m, 3H), 7.42-7.40 (m, 1H), 7.30 (d, J=7.2 Hz, 1H), 7.14-7.14 (m, 1H), 4.06-3.88 (m, 12H), 3.77 (s, 3H), 3.61-3.53 (m, 1H), 3.13 (s, 3H), 2.94-2.84 (m, 2H), 2.40-2.33 (m, 3H), 1.92-1.83 (m, 1H).

### Example 294: (S)-Methyl 2-(((8-(2,2′-dichloro-3′-(6-methoxy-5-((((5-oxopyrrolidin-2-yl)methyl)amino)methyl)pyridin-2-yl)-[1,1′-biphenyl]-3-yl)-6-methyl-4-oxo-4H-pyrido[1,2-a]pyrimidin-3-yl)methyl)amino)acetate

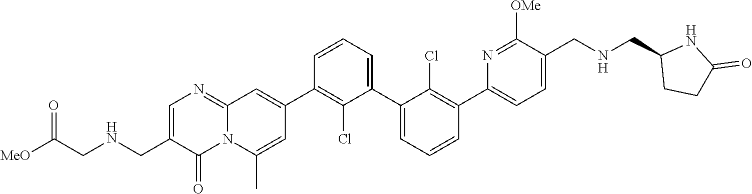 [SMILES: COC(=O)CNCc1cnc2cc(-c3cccc(-c4cccc(-c5ccc(CNC[C@@H]6CCC(=O)N6)c(OC)n5)c4Cl)c3Cl)cc(C)n2c1=O]

This compound was prepared in a similar fashion to Example 226, using methyl 2-aminoacetate HCl and tert-butyl (S)-((6-(2,2′-dichloro-3′-(3-formyl-6-methyl-4-oxo-4H-pyrido[1,2-a]pyrimidin-8-yl)-[1,1′-biphenyl]-3-yl)-2-methoxypyridin-3-yl)methyl)((5-oxopyrrolidin-2-yl)methyl)carbamate. MS: m/z found 715 [M+H]⁺, (Method A). ¹H NMR (400 MHz, Methanol-d₄): δ8.24 (s, 1H), 7.83 (d, J=7.6 Hz, 1H), 7.68 (dd, J=7.6 Hz, J=1.6 Hz, 1H), 7.61-7.59 (m, 2H), 7.56-7.50 (m, 3H), 7.43-7.41 (m, 1H), 7.14 (s, 1H), 4.08 (s, 5H), 3.98-3.97 (m, 3H), 3.78 (s, 3H), 3.74 (s, 2H), 3.15 (s, 3H), 2.99-2.96 (m, H), 2.42-2.38 (m, 3H), 1.91-1.84 (m, 1H).

### Example 295: (S)-Methyl 1-((8-(2,2′-dichloro-3′-(6-methoxy-5-(((((S)-5-oxopyrrolidin-2-yl)methyl)amino)methyl)pyridin-2-yl)-[1,1′-biphenyl]-3-yl)-6-methyl-4-oxo-4H-pyrido[1,2-a]pyrimidin-3-yl)methyl)pyrrolidine-3-carboxylate

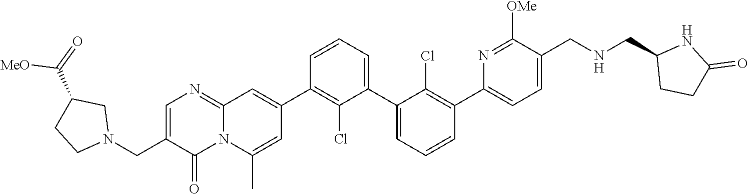 [SMILES: COC(=O)[C@H]1CCN(Cc2cnc3cc(-c4cccc(-c5cccc(-c6ccc(CNC[C@@H]7CCC(=O)N7)c(OC)n6)c5Cl)c4Cl)cc(C)n3c2=O)C1]

This compound was prepared in a similar fashion to Example 226, using methyl (3S)-pyrrolidine-3-carboxylate HCl and tert-butyl (S)-((6-(2,2′-dichloro-3′-(3-formyl-6-methyl-4-oxo-4H-pyrido[1,2-a]pyrimidin-8-yl)-[1,1′-biphenyl]-3-yl)-2-methoxypyridin-3-yl)methyl)((5-oxopyrrolidin-2-yl)methyl)carbamate. MS: m/z found 755 [M+H]⁺, (Method AH). ¹H NMR (400 MHz, DMSO-d₆): δ8.18 (s, 1H), 8.15 (s, 1H), 7.86 (d, J=7.2 Hz, 1H), 7.70-7.68 (m, 2H), 7.66-7.58 (m, 2H), 7.57-7.54 (m, 2H), 7.50-7.45 (m, 2H), 7.30 (d, J=7.6 Hz, 1H), 7.10 (s, 1H), 3.94 (s, 3H), 3.85-3.81 (m, 2H), 3.70-3.69 (m, 2H), 3.67-3.62 (m, 4H), 3.11-3.05 (m, 2H), 3.01 (s, 3H), 2.89-2.87 (m, 1H), 2.81-2.79 (m, 1H), 2.68-2.65 (m, 4H), 2.19-1.95 (m, 5H), 1.77-1.69 (m, 1H).

### Example 308: (S)-8-(2,2′-Dichloro-3′-(6-methoxy-5-((((5-oxopyrrolidin-2-yl)methyl) amino)methyl)pyridin-2-yl)-[1,1′-biphenyl]-3-yl)-3-(((2-hydroxyethyl)amino)methyl)-6-methyl-4H-pyrido[1,2-a]pyrimidin-4-one

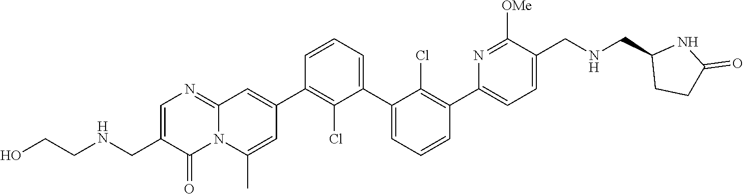 [SMILES: COc1nc(-c2cccc(-c3cccc(-c4cc(C)n5c(=O)c(CNCCO)cnc5c4)c3Cl)c2Cl)ccc1CNC[C@@H]1CCC(=O)N1]

This compound was prepared in a similar fashion to Example 226, using 2-aminoethanol and tert-butyl (S)-((6-(2,2′-dichloro-3′-(3-formyl-6-methyl-4-oxo-4H-pyrido[1,2-a]pyrimidin-8-yl)-[1,1′-biphenyl]-3-yl)-2-methoxypyridin-3-yl)methyl)((5-oxopyrrolidin-2-yl)methyl)carbamate. MS: m/z found 687 [M+H]⁺, (Method AG). ¹H NMR (400 MHz, Methanol-d₄): δ8.19 (s, 1H), 7.73 (d, J=7.6 Hz, 1H), 7.65 (dd, J=9.2 Hz, J=6.4 Hz, 1H), 7.58-7.57 (m, 2H), 7.52-7.47 (m, 3H), 7.38 (dd, J=9.2 Hz, J=6.0 Hz, 1H), 7.24 (d, J=7.6 Hz, 1H), 7.08 (s, 1H), 4.02 (s, 3H), 3.87-3.79 (m, 5H), 3.73-3.70 (m, 2H), 3.11 (s, 3H), 2.84-2.81 (m, 2H), 2.74-2.65 (m, 2H), 2.38-2.23 (m, 3H), 1.86-1.77 (m, 1H).

### Example 323: 8-(2,2′-Dichloro-3′-(6-methoxy-5-(((((S)-5-oxopyrrolidin-2-yl)methyl)amino) methyl)pyridin-2-yl)-[1,1′-biphenyl]-3-yl)-3-(5-hydroxy-1,4,5,6-tetrahydropyrimidin-2-yl)-6-methyl-4H-pyrido[1,2-a]pyrimidin-4-one

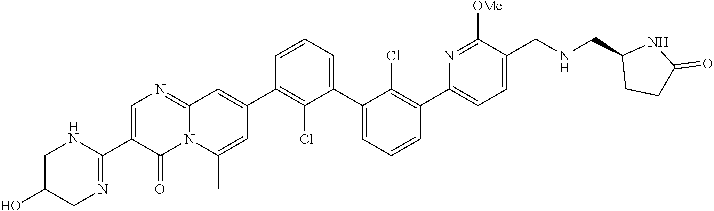 [SMILES: COc1nc(-c2cccc(-c3cccc(-c4cc(C)n5c(=O)c(C6=NCC(O)CN6)cnc5c4)c3Cl)c2Cl)ccc1CNC[C@@H]1CCC(=O)N1]

This compound was prepared in a similar fashion to Example 290, using 1,3-diaminopropan-2-ol and (S)-tert-butyl ((6-(2,2′-dichloro-3′-(3-formyl-6-methyl-4-oxo-4H-pyrido[1,2-a]pyrimidin-8-yl)-[1,1′-biphenyl]-3-yl)-2-methoxypyridin-3-yl)methyl)((5-oxopyrrolidin-2-yl)methyl) carbamate. MS: m/z found 712 [M+H]⁺, (Method A). ¹H NMR (400 MHz, Methanol-d₄) δ 8.74 (s, 1H), 7.81 (m, 2H), 7.69-7.63 (m, 3H), 7.56-7.51 (m, 2H), 7.46 (s, 1H), 7.42 (dd, J=1.6 Hz, J=7.2 Hz, 1H), 7.29 (d, J=7.2 Hz, 1H), 4.39 (t, J=3.2 Hz, 1H), 4.06-3.92 (m, 6H), 3.71-3.67 (m, 2H), 3.58-3.54 (m, 2H), 3.19 (s, 3H), 2.90-2.84 (m, 2H), 2.42-2.30 (m, 3H), 1.95-1.84 (m, 1H).

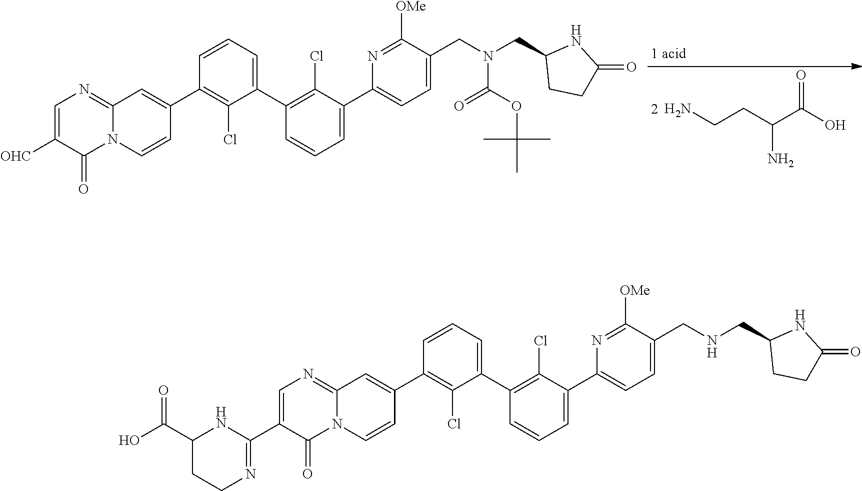 [SMILES: COc1nc(-c2cccc(-c3cccc(-c4ccn5c(=O)c(C=O)cnc5c4)c3Cl)c2Cl)ccc1CN(C[C@@H]1CCC(=O)N1)C(=O)OC(C)(C)C ; NCCC(N)C(=O)O ; COc1nc(-c2cccc(-c3cccc(-c4ccn5c(=O)c(C6=NCCC(C(=O)O)N6)cnc5c4)c3Cl)c2Cl)ccc1CNC[C@@H]1CCC(=O)N1]

### Example 199: 2-(8-(2,2′-Dichloro-3′-(6-methoxy-5-(((((S)-5-oxopyrrolidin-2-yl)methyl) amino)methyl)pyridin-2-yl)-[1,1′-biphenyl]-3-yl)-4-oxo-4H-pyrido[1,2-a]pyrimidin-3-yl)-3,4,5,6-tetrahydropyrimidine-4-carboxylic acid

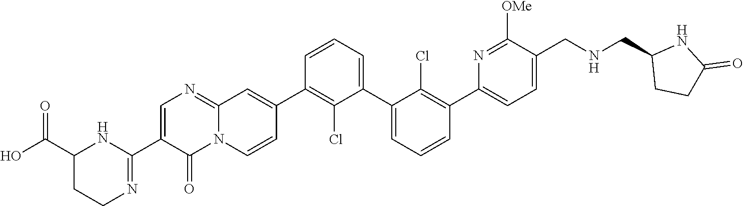 [SMILES: COc1nc(-c2cccc(-c3cccc(-c4ccn5c(=O)c(C6=NCCC(C(=O)O)N6)cnc5c4)c3Cl)c2Cl)ccc1CNC[C@@H]1CCC(=O)N1]

### (S)-8-(2,2′-Dichloro-3′-(6-methoxy-5-((((5-oxopyrrolidin-2-yl)methyl) amino) methyl)pyridin-2-yl)-[1,1′-biphenyl]-3-yl)-4-oxo-4H-pyrido[1,2-a]pyrimidine-3-carbaldehyde

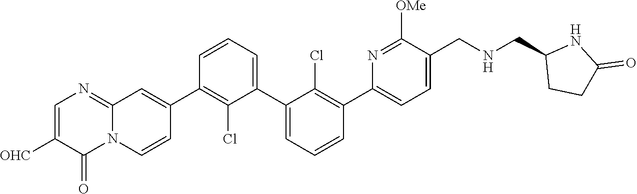 [SMILES: COc1nc(-c2cccc(-c3cccc(-c4ccn5c(=O)c(C=O)cnc5c4)c3Cl)c2Cl)ccc1CNC[C@@H]1CCC(=O)N1]

To a mixture of tert-butyl (S)-tert-butyl ((6-(2,2′-dichloro-3′-(3-formyl-4-oxo-4H-pyrido[1,2-a]pyrimidin-8-yl)-[1,1′-biphenyl]-3-yl)-2-methoxypyridin-3-yl)methyl)((5-oxopyrrolidin-2-yl)methyl)carbamate in DCM (15 mL) was added trifluoroacetic acid (1.75 mL, 23.6 mmol). The mixture was stirred at r.t. for 0.5 h under N₂. The mixture was concentrated to afford (S)-8-(2,2′-dichloro-3′-(6-methoxy-5-((((5-oxopyrrolidin-2-yl)methyl)amino)methyl)pyridin-2-yl)-[1,1′-biphenyl]-3-yl)-4-oxo-4H-pyrido[1,2-a]pyrimidine-3-carbaldehyde as a crude yellow oil (350 mg, m/z: 628 [M+H]⁺ observed (Method AD).

### 2-(8-(2,2′-Dichloro-3′-(6-methoxy-5-(((((S)-5-oxopyrrolidin-2-yl)methyl) amino) methyl)pyridin-2-yl)-[1,1′-biphenyl]-3-yl)-4-oxo-4H-pyrido[1,2-a]pyrimidin-3-yl)-3,4,5,6-tetrahydropyrimidine-4-carboxylic acid

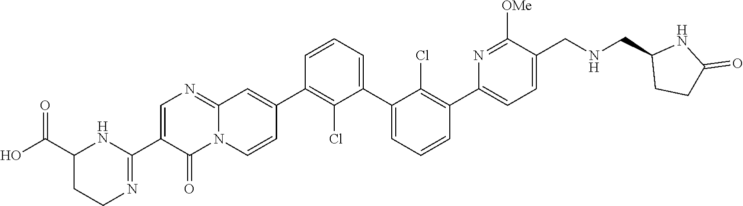 [SMILES: COc1nc(-c2cccc(-c3cccc(-c4ccn5c(=O)c(C6=NCCC(C(=O)O)N6)cnc5c4)c3Cl)c2Cl)ccc1CNC[C@@H]1CCC(=O)N1]

To a mixture of (S)-8-(2,2′-dichloro-3′-(6-methoxy-5-((((5-oxopyrrolidin-2-yl)methyl) amino)methyl)pyridin-2-yl)-[1,1′-biphenyl]-3-yl)-4-oxo-4H-pyrido[1,2-a]pyrimidine-3-carbaldehyde (350 mg, 0.56 mmol), 2,4-diaminobutanoic acid HCl (172 mg, 1.11 mmol) in DMA (3 mL) was added potassium carbonate (770 mg, 5.57 mmol), iodine (848 mg, 3.34 mmol), and then the mixture was stirred at r.t. for 12 h under N₂. Reaction was concentrated, and the residue was purified by reverse phase HPLC to afford 2-(8-(2,2′-dichloro-3′-(6-methoxy-5-(((((S)-5-oxopyrrolidin-2-yl)methyl)amino)methyl)pyridin-2-yl)-[1,1′-biphenyl]-3-yl)-4-oxo-4H-pyrido[1,2-a]pyrimidin-3-yl)-3,4,5,6-tetrahydropyrimidine-4-carboxylic acid (23.3 mg, m/z: 726 [M+H]⁺ observed, Method A) as a white solid. ¹H NMR (400 MHz, Methanol-d₄): δ 9.34 (d, J=7.2 Hz, 1H), 8.97 (s, 1H), 8.02 (s, 1H), 7.81 (d, J=7.2 Hz, 1H), 7.72 (d, J=8 Hz, 1H), 7.67-7.62 (m, 3H), 7.55-7.48 (m, 2H), 7.40 (d, J=1.2 Hz, 1H), 7.24-7.22 (m, 1H), 4.19-4.16 (m, 1H), 4.09 (s, 3H), 3.86-3.81 (m, 3H), 3.65-3.62 (m, 2H), 2.70-2.67 (m, 2H), 2.35-2.18 (m, 5H), 1.82-1.80 (m, 1H).

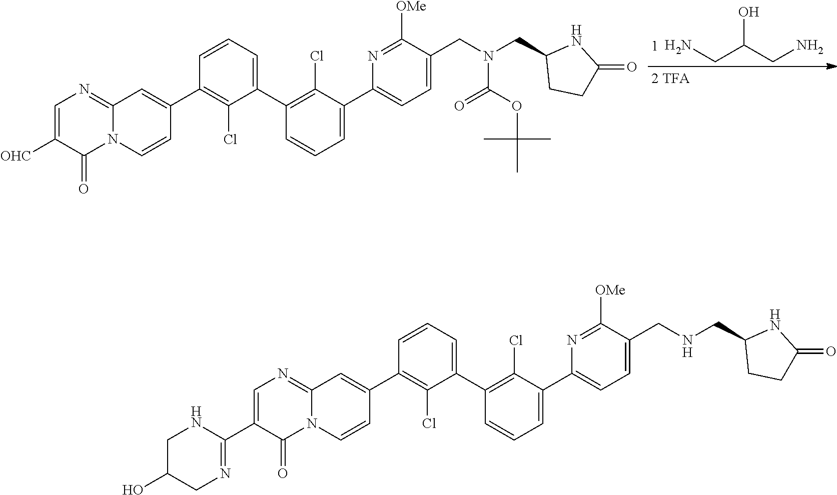 [SMILES: COc1nc(-c2cccc(-c3cccc(-c4ccn5c(=O)c(C=O)cnc5c4)c3Cl)c2Cl)ccc1CN(C[C@@H]1CCC(=O)N1)C(=O)OC(C)(C)C ; NCC(O)CN ; COc1nc(-c2cccc(-c3cccc(-c4ccn5c(=O)c(C6=NCC(O)CN6)cnc5c4)c3Cl)c2Cl)ccc1CNC[C@@H]1CCC(=O)N1]

### Example 161: 8-(2,2′-Dichloro-3′-(6-methoxy-5-(((((S)-5-oxopyrrolidin-2-yl)methyl)amino)methyl)pyridin-2-yl)-[1,1′-biphenyl]-3-yl)-3-(5-hydroxy-1,4,5,6-tetrahydropyrimidin-2-yl)-4H-pyrido[1,2-a]pyrimidin-4-one

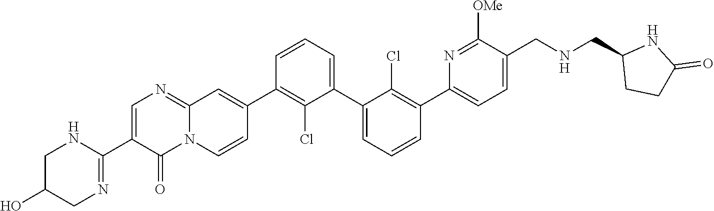 [SMILES: COc1nc(-c2cccc(-c3cccc(-c4ccn5c(=O)c(C6=NCC(O)CN6)cnc5c4)c3Cl)c2Cl)ccc1CNC[C@@H]1CCC(=O)N1]

### tert-Butyl ((6-(2,2′-dichloro-3′-(3-(5-hydroxy-1,4,5,6-tetrahydropyrimidin-2-yl)-4-oxo-4H-pyrido[1,2-a]pyrimidin-8-yl)-[1,1′-biphenyl]-3-yl)-2-methoxypyridin-3-yl)methyl)(((S)-5-oxopyrrolidin-2-yl)methyl)carbamate

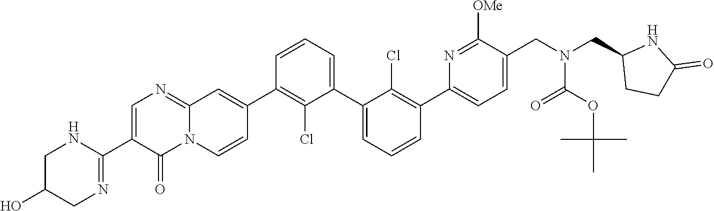 [SMILES: COc1nc(-c2cccc(-c3cccc(-c4ccn5c(=O)c(C6=NCC(O)CN6)cnc5c4)c3Cl)c2Cl)ccc1CN(C[C@@H]1CCC(=O)N1)C(=O)OC(C)(C)C]

To a mixture of (S)-tert-butyl ((6-(2,2′-dichloro-3′-(3-formyl-4-oxo-4H-pyrido[1,2-a]pyrimidin-8-yl)-[1,1′-biphenyl]-3-yl)-2-methoxypyridin-3-yl)methyl)((5-oxopyrrolidin-2-yl)methyl)carbamate (0.6 g, 0.82 mmol) and 1,3-diaminopropan-2-ol (0.22 g, 2.47 mmol) in DMA (8 mL) was added potassium carbonate (0.8 g, 5.76 mmol) and iodine (1.25 g, 4.94 mmol) in one portion under N₂ atmosphere. The mixture was stirred at 35° C. for 16 h. Water was added (20 mL), then a saturated aqueous sodium sulfite solution (40 mL) and saturated aqueous brine solution (20 mL). The mixture was extracted with ethyl acetate/THF mixture (1:1, 2×200 mL). The organic phase was dried with anhydrous sodium sulfate, filtered and concentrated to afford tert-butyl ((6-(2,2′-dichloro-3′-(3-(5-hydroxy-1,4,5,6-tetrahydropyrimidin-2-yl)-4-oxo-4H-pyrido[1,2-a]pyrimidin-8-yl)-[1,1′-biphenyl]-3-yl)-2-methoxypyridin-3-yl)methyl)(((S)-5-oxopyrrolidin-2-yl)methyl)carbamate as a brown gum (0.65 g, crude, m/z: 798 [M+H]⁺ observed, Method AF).

### 8-(2,2′-Dichloro-3′-(6-methoxy-5-(((((S)-5-oxopyrrolidin-2-yl)methyl)amino)methyl) pyridin-2-yl)-[1,1′-biphenyl]-3-yl)-3-(5-hydroxy-1,4,5,6-tetrahydropyrimidin-2-yl)-4H-pyrido[1,2-a]pyrimidin-4-one

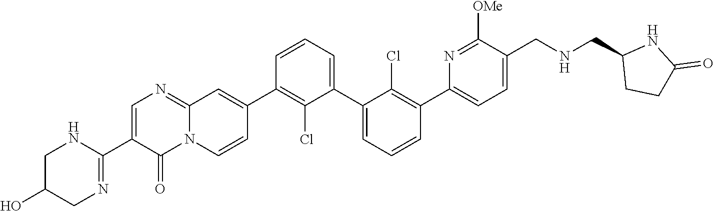 [SMILES: COc1nc(-c2cccc(-c3cccc(-c4ccn5c(=O)c(C6=NCC(O)CN6)cnc5c4)c3Cl)c2Cl)ccc1CNC[C@@H]1CCC(=O)N1]

To a mixture of tert-butyl ((6-(2,2′-dichloro-3′-(3-(5-hydroxy-1,4,5,6-tetrahydropyrimidin-2-yl)-4-oxo-4H-pyrido[1,2-a]pyrimidin-8-yl)-[1,1′-biphenyl]-3-yl)-2-methoxypyridin-3-yl)methyl)(((S)-5-oxopyrrolidin-2-yl)methyl)carbamate (0.6 g, 0.75 mmol) in THE (0.5 mL), was added trifluoroacetic acid (4 mL, 54 mmol), and the reaction was stirred at r.t. for 2 h, then at 40° C. for 1 h. The mixture was concentrated, and the residue was purified by reverse phase HPLC to afford 8-(2,2′-dichloro-3′-(6-methoxy-5-(((((S)-5-oxopyrrolidin-2-yl)methyl)amino) methyl)pyridin-2-yl)-[1,1′-biphenyl]-3-yl)-3-(5-hydroxy-1,4,5,6-tetrahydropyrimidin-2-yl)-4H-pyrido[1,2-a]pyrimidin-4-one as a yellow solid (25.3 mg, 4% yield, m/z: 698 [M+H]⁺ observed, Method AF). ¹H NMR (400 MHz, CDCl₃): δ 9.38 (s, 1H), 9.13 (d, J=7.2 Hz, 1H), 7.89 (d, J=1.6 Hz, 1H), 7.64-7.62 (m, 1H), 7.53 (d, J=7.6 Hz, 2H), 7.46-7.37 (m, 4H), 7.27-7.25 (m, 1H), 7.21 (m, 1H), 5.91 (s, 1H), 4.23 (s, 1H), 3.96 (s, 3H), 3.74 (s, 2H), 3.70-3.52 (m, 5H), 2.76-2.72 (m, 1H), 2.53-2.50 (m, 1H), 2.32-2.27 (m, 2H), 2.20-2.15 (m, 1H), 1.73-1.66 (m, 2H), 1.22-1.16 (m, 1H), 0.77 (m, 1H).

### Example 167: 8-(2,2′-Dichloro-3′-(6-methoxy-5-(((((S)-5-oxopyrrolidin-2-yl)methyl)amino) methyl)pyridin-2-yl)-[1,1′-biphenyl]-3-yl)-3-(5-hydroxy-1,4,5,6-tetrahydropyrimidin-2-yl)-4H-pyrido[1,2-a]pyrimidin-4-one

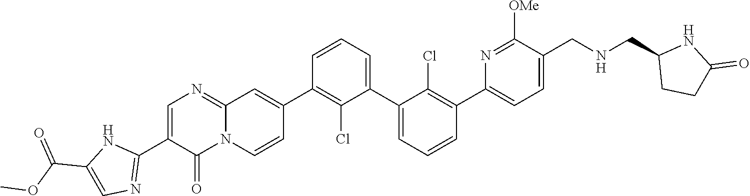 [SMILES: COC(=O)c1cnc(-c2cnc3cc(-c4cccc(-c5cccc(-c6ccc(CNC[C@@H]7CCC(=O)N7)c(OC)n6)c5Cl)c4Cl)ccn3c2=O)[nH]1]

### Methyl 2,3-diaminopropanoate

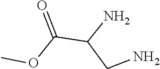

To a solution of 2,3-diaminopropanoic acid as hydrochloride salt (1 g, 7.11 mmol) in MeOH (15 mL) was added sulfurous dichloride (2.58 mL, 35.57 mmol) at rt. The mixture was stirred at 60° C. for 12 hr. The reaction was concentrated to give the crude product methyl 2,3-diaminopropanoate as hydrochloride salt as a white solid (1.4 g, crude) which was used into the next step without further purification. ¹H NMR (400 MHz, Methanol-d₄): δ 4.51-4.47 (m, 1H), 3.96 (s, 3H), 3.55-3.33 (s, 2H).

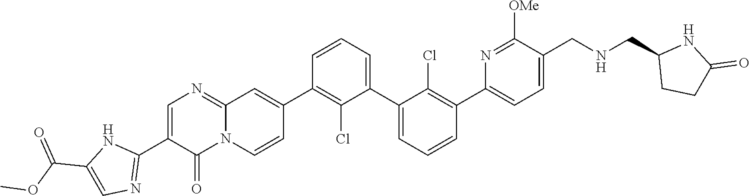 [SMILES: COC(=O)c1cnc(-c2cnc3cc(-c4cccc(-c5cccc(-c6ccc(CNC[C@@H]7CCC(=O)N7)c(OC)n6)c5Cl)c4Cl)ccn3c2=O)[nH]1]

This compound was prepared in a similar fashion to Example 161, using methyl 2,3-diaminopropanoate and tert-butyl ((6-(2,2′-dichloro-3′-(3-(5-hydroxy-1,4,5,6-tetrahydropyrimidin-2-yl)-4-oxo-4H-pyrido[1,2-a]pyrimidin-8-yl)-[1,1′-biphenyl]-3-yl)-2-methoxypyridin-3-yl)methyl)(((S)-5-oxopyrrolidin-2-yl)methyl)carbamate. MS: m/z found 724 [M+H]⁺, (Method A). ¹H NMR (400 MHz, Methanol-d₄): δ 9.34-9.32 (m, 2H), 7.93 (s, 1H), 7.92 (s, 1H), 7.83 (d, J=7.6 Hz, 1H), 7.69-7.65 (m, 4H), 7.63-7.53 (m, 2H), 7.45-7.43 (dd, J=2.0 Hz, J=7.6 Hz, 1H), 7.32 (d, J=7.6 Hz, 1H), 4.10-4.08 (m, 5H), 3.99-3.93 (m, 4H), 3.01-2.98 (m, 2H), 2.42-2.34 (m, 3H), 1.94-1.85 (m, 1H).

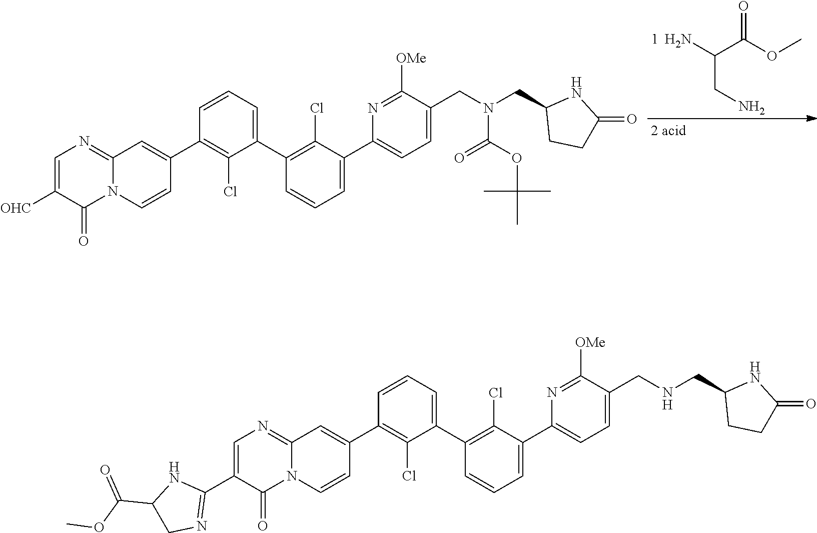

### Example 171: Methyl 2-(8-(2,2′-dichloro-3′-(6-methoxy-5-(((((S)-5-oxopyrrolidin-2-yl)methyl)amino)methyl)pyridin-2-yl)-[1,1′-biphenyl]-3-yl)-4-oxo-4H-pyrido[1,2-a]pyrimidin-3-yl)-4,5-dihydro-1H-imidazole-5-carboxylate

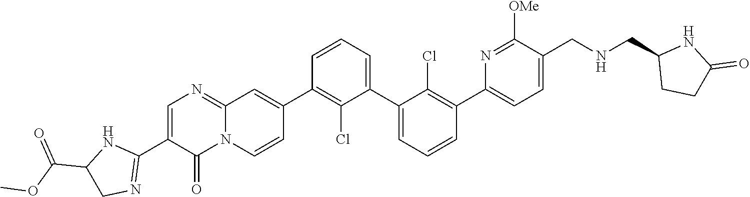 [SMILES: COC(=O)C1CN=C(c2cnc3cc(-c4cccc(-c5cccc(-c6ccc(CNC[C@@H]7CCC(=O)N7)c(OC)n6)c5Cl)c4Cl)ccn3c2=O)N1]

### Methyl 2-(8-(3′-(5-(((tert-butoxycarbonyl)(((S)-5-oxopyrrolidin-2-yl)methyl)amino)methyl)-6-methoxypyridin-2-yl)-2,2′-dichloro-[1,1′-biphenyl]-3-yl)-4-oxo-4H-pyrido[1,2-a]pyrimidin-3-yl)-4,5-dihydro-1H-imidazole-5-carboxylate

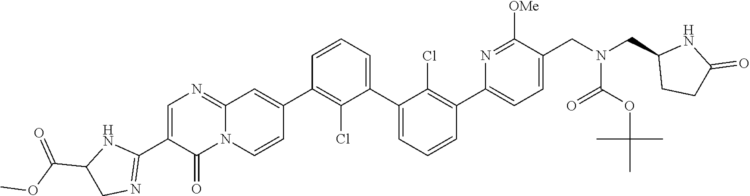 [SMILES: COC(=O)C1CN=C(c2cnc3cc(-c4cccc(-c5cccc(-c6ccc(CN(C[C@@H]7CCC(=O)N7)C(=O)OC(C)(C)C)c(OC)n6)c5Cl)c4Cl)ccn3c2=O)N1]

To a solution of (S)-tert-butyl ((6-(2,2′-dichloro-3′-(3-formyl-4-oxo-4H-pyrido[1,2-a]pyrimidin-8-yl)-[1,1′-biphenyl]-3-yl)-2-methoxypyridin-3-yl)methyl)((5-oxopyrrolidin-2-yl)methyl)carbamate (500 mg, 0.69 mmol) and methyl 2,3-diaminopropanoate HCl (127 mg, 0.82 mmol) in DCM (10 mL) was added potassium carbonate (474 mg, 3.43 mmol). The reaction was stirred for 2 hr at 0° C., then 1-bromopyrrolidine-2,5-dione (147 mg, 0.82 mmol) was added. The mixture was stirred at r.t. for 12 hr, then H₂O was added (20 mL), then mixture was extracted with DCM (60 mL). The organic layer was dried over anhydrous sodium sulfate, filtered and concentrated. The crude product was triturated with EtOAc (20 mL) at r.t. for 30 min. The filtrate was concentrated in vacuum to give methyl 2-(8-(3′-(5-(((tert-butoxy carbonyl)(((S)-5-oxopyrrolidin-2-yl)methyl)amino)methyl)-6-methoxy pyridin-2-yl)-2,2′-dichloro-[1,1′-biphenyl]-3-yl)-4-oxo-4H-pyrido[1,2-a]pyrimidin-3-yl)-4,5-dihydro-1H-imidazole-5-carboxylate a dark red oil (500 mg, crude), which was used into the next step without further purification.

### Methyl 2-(8-(2,2′-dichloro-3′-(6-methoxy-5-(((((S)-5-oxopyrrolidin-2-yl)methyl)amino) methyl)pyridin-2-yl)-[1,1′-biphenyl]-3-yl)-4-oxo-4H-pyrido[1,2-a]pyrimidin-3-yl)-4,5-dihydro-1H-imidazole-5-carboxylate

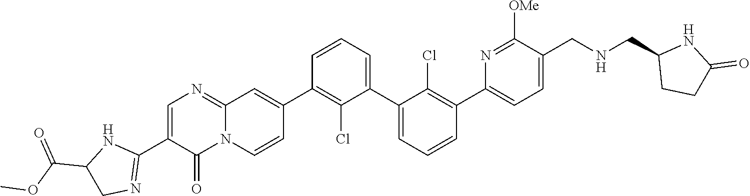 [SMILES: COC(=O)C1CN=C(c2cnc3cc(-c4cccc(-c5cccc(-c6ccc(CNC[C@@H]7CCC(=O)N7)c(OC)n6)c5Cl)c4Cl)ccn3c2=O)N1]

To the mixture of methyl 2-(8-(3′-(5-(((tert-butoxycarbonyl)(((S)-5-oxopyrrolidin-2-yl)methyl) amino)methyl)-6-methoxypyridin-2-yl)-2,2′-dichloro-[1,1′-biphenyl]-3-yl)-4-oxo-4H-pyrido[1,2-a]pyrimidin-3-yl)-4,5-dihydro-1H-imidazole-5-carboxylate (200 mg, 0.24 mmol) in DCM (3 mL) was added trifluoroacetic acid (7.50 mL, 101.30 mmol), the reaction was stirred for 0.5 hr at rt. The mixture was concentrated, and then purified by reverse phase HPLC to afford methyl 2-(8-(2,2′-dichloro-3′-(6-methoxy-5-(((((S)-5-oxop yrrolidin-2-yl)methyl)amino)methyl)pyridin-2-yl)-[1,1′-biphenyl]-3-yl)-4-oxo-4H-pyrido[1,2-a]pyrimidin-3-yl)-4,5-dihydro-1H-imidazole-5-carboxylate as formic acid salt as a yellow solid (31 mg, 15% yield). m/z: 726 [M]⁺ observed (Method A). ¹H NMR (400 MHz, Methanol-d₄): δ 9.42 (d, J=7.2 Hz, 1H), 9.07 (s, 1H), 8.12 (s, 1H), 7.92 (d, J=6.8 Hz, 1H), 7.86 (d, J=6.8 Hz, 1H), 7.71-7.65 (m, 3H), 7.59-7.54 (m, 2H), 7.45 (d, J=7.2 Hz, 1H), 7.34 (d, J=7.6 Hz, 1H), 5.11-5.06 (dd, J=6.8 Hz, J=12 Hz, 1H), 4.38-4.25 (m, 2H), 4.18 (s, 2H), 4.09 (s, 3H), 4.06-4.02 (m, 1H), 3.89 (s, 3H), 3.08 (br s, 2H), 2.43-2.34 (m, 3H), 1.91-1.90 (m, 1H).

### Example 179: 2-(8-(2,2′-Dichloro-3′-(6-methoxy-5-(((((S)-5-oxopyrrolidin-2-yl)methyl)amino)methyl)pyridin-2-yl)-[1,1′-biphenyl]-3-yl)-4-oxo-4H-pyrido[1,2-a]pyrimidin-3-yl)-4,5-dihydro-1H-imidazole-5-carboxylic acid

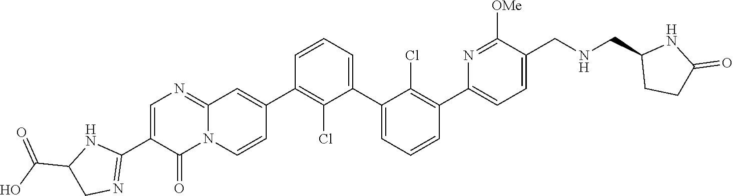 [SMILES: COc1nc(-c2cccc(-c3cccc(-c4ccn5c(=O)c(C6=NCC(C(=O)O)N6)cnc5c4)c3Cl)c2Cl)ccc1CNC[C@@H]1CCC(=O)N1]

This compound was prepared in a similar fashion to Example 161, using 2,3-diaminopropanoic acid and tert-butyl ((6-(2,2′-dichloro-3′-(3-(5-hydroxy-1,4,5,6-tetrahydropyrimidin-2-yl)-4-oxo-4H-pyrido[1,2-a]pyrimidin-8-yl)-[1,1′-biphenyl]-3-yl)-2-methoxypyridin-3-yl)methyl)(((S)-5-oxopyrrolidin-2-yl)methyl)carbamate. MS: m/z found 712 [M+H]⁺, (Method A). ¹H NMR (400 MHz, DMSO-d₆): δ 9.33 (d, J=7.2 Hz, 1H), 9.16 (s, 1H), 8.17 (d, J=2.0 Hz, 1H), 7.94-7.89 (m, 2H), 7.75-7.59 (m, 5H), 7.52 (d, J=6.0 Hz, 1H), 7.37 (d, J=7.6 Hz, 1H), 4.24-4.18 (m, 1H), 4.10-4.05 (m, 3H), 3.98 (s, 3H), 3.83-3.79 (m, 1H), 2.99-2.89 (m, 3H), 2.23-2.24 (m, 3H), 1.79-1.75 (m, 1H).

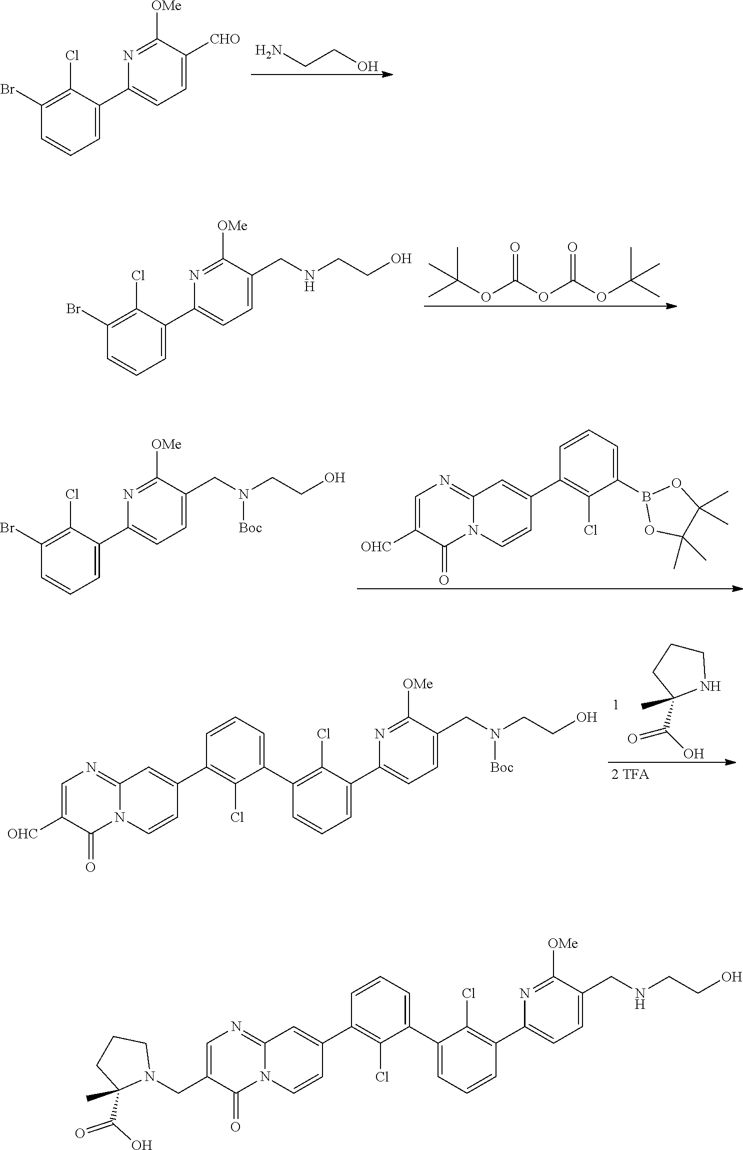

### Example 168: 2-(8-(2,2′-Dichloro-3′-(6-methoxy-5-(((((S)-5-oxopyrrolidin-2-yl)methyl) amino)methyl)pyridin-2-yl)-[1,1′-biphenyl]-3-yl)-4-oxo-4H-pyrido[1,2-a]pyrimidin-3-yl)-4,5-dihydro-1H-imidazole-5-carboxylic acid

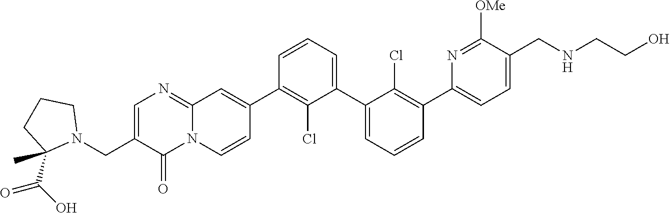 [SMILES: COc1nc(-c2cccc(-c3cccc(-c4ccn5c(=O)c(CN6CCC[C@@]6(C)C(=O)O)cnc5c4)c3Cl)c2Cl)ccc1CNCCO]

### 2-(((6-(3-Bromo-2-chlorophenyl)-2-methoxypyridin-3-yl)methyl)amino)ethanol

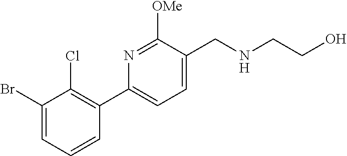 [SMILES: COc1nc(-c2cccc(Br)c2Cl)ccc1CNCCO]

To a solution of 6-(3-bromo-2-chlorophenyl)-2-methoxynicotinaldehyde (3 g, 9.19 mmol) and 2-aminoethanol (0.83 mL, 13.8 mmol) in DCM (80 mL) was added sodium acetate (1.51 g, 18.4 mmol). The reaction was stirred at r.t. for 6 h. Sodium triacetoxyborohydride (2.92 g, 13.8 mmol) was added, the mixture was stirred at r.t. for 1 hr. Crude 2-(((6-(3-bromo-2-chlorophenyl)-2-methoxy pyridin-3-yl)methyl)amino)ethanol (3.41 g, crude) was obtained as a yellow DCM solution (80 mL).

### tert-Butyl ((6-(3-bromo-2-chlorophenyl)-2-methoxypyridin-3-yl)methyl)(2-hydro xyethyl)carbamate

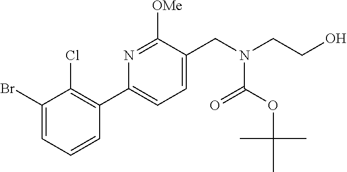 [SMILES: COc1nc(-c2cccc(Br)c2Cl)ccc1CN(CCO)C(=O)OC(C)(C)C]

To a solution of 2-(((6-(3-bromo-2-chlorophenyl)-2-methoxypyridin-3-yl)methyl) amino) ethanol (3.41 g, 9.18 mmol) in DCM (80 mL) was added triethylamine (3.83 mL, 27.5 mmol) and di-tert-butyl dicarbonate (6.01 g, 27.5 mmol). The reaction was stirred at r.t. for 2 hr, then quenched with H₂O (50 mL) and extracted with DCM (60 mL). The organic layer was dried over anhydrous sodium sulfate, filtered and the filtrate was concentrated. The residue was purified by normal phase SiO₂ chromatography (0-40% EtOAc/petroleum ether) to afford tert-butyl ((6-(3-bromo-2-chlorophenyl)-2-methoxypyridin-3-yl)methyl)(2-hydroxyethyl)carbamate as a yellow oil (3 g, 56.42% yield). ¹H NMR (400 MHz, CDCl₃): δ 7.59 (d, J=6.8 Hz, 1H), 7.46-7.42 (m, 2H), 7.13-7.08 (m, 2H), 4.40 (s, 2H), 3.91 (s, 3H), 3.71 (m, 2H), 3.44 (s, 2H), 1.17 (s, 9H).

### tert-Butyl ((6-(2,2′-dichloro-3′-(3-formyl-4-oxo-4H-pyrido[1,2-a]pyrimidin-8-yl)-[1,1′-biphenyl]-3-yl)-2-methoxypyridin-3-yl)methyl)(2-hydroxyethyl)carbamate

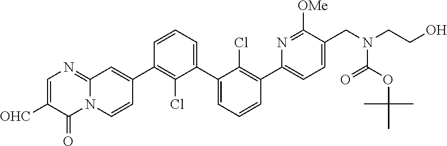 [SMILES: COc1nc(-c2cccc(-c3cccc(-c4ccn5c(=O)c(C=O)cnc5c4)c3Cl)c2Cl)ccc1CN(CCO)C(=O)OC(C)(C)C]

To a mixture of tert-butyl ((6-(3-bromo-2-chlorophenyl)-2-methoxypyridin-3-yl)methyl) (2-hydroxyethyl)carbamate (1.5 g, 3.18 mmol) and 8-(2-chloro-3-(4,4,5,5-tetramethyl-1,3,2-dioxaborolan-2-yl)phenyl)-4-oxo-4H-pyrido[1,2-a]pyrimidine-3-carbaldehyde (1.44 g, 3.50 mmol) in dioxane/H₂O (10:1, 55 mL) was added 1,1′-bis(di-tert-butylphosphino)ferrocene palladium dichloride (207 mg, 0.32 mmol), followed by potassium phosphate (2.02 g, 9.54 mmol). The reaction was stirred for 0.5 hr at 110° C. After cooling, the mixture was added H₂O (100 mL) and extracted with EtOAc (2×100 mL). The organic layer was concentrated, and the residue was purified by normal phase SiO₂ chromatography (0-60% EtOAc/30% THE in petroleum ether) to afford tert-butyl ((6-(2,2′-dichloro-3′-(3-formyl-4-oxo-4H-pyrido[1,2-a]pyrimidin-8-yl)-[1,1′-biphenyl]-3-yl)-2-methoxypyridin-3-yl)methyl)(2-hydroxyethyl)carbamate as an orange solid (0.9 g, 41% yield). ¹H NMR (400 MHz, CDCl₃): δ 9.24 (d, J=7.2 Hz, 1H), 8.88 (s, 1H), 7.89 (s, 1H), 7.88-7.22 (m, 9H), 4.42 (s, 2H), 3.95 (s, 3H), 3.70 (m, 3H), 3.45 (br s, 2H), 1.38 (s, 9H).

### (S)-1-((8-(3′-(5-(((tert-Butoxycarbonyl)(2-hydroxyethyl)amino)methyl)-6-methoxypyridin-2-yl)-2,2′-dichloro-[1,1′-biphenyl]-3-yl)-4-oxo-4H-pyrido[1,2-a]pyrimidin-3-yl)methyl)-2-methylpyrrolidine-2-carboxylic acid

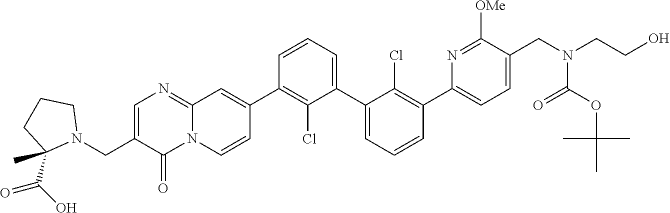 [SMILES: COc1nc(-c2cccc(-c3cccc(-c4ccn5c(=O)c(CN6CCC[C@@]6(C)C(=O)O)cnc5c4)c3Cl)c2Cl)ccc1CN(CCO)C(=O)OC(C)(C)C]

To the mixture of tert-butyl ((6-(2,2′-dichloro-3′-(3-formyl-4-oxo-4H-pyrido[1,2-a]pyrimidin-8-yl)-[1,1′-biphenyl]-3-yl)-2-methoxypyridin-3-yl)methyl)(2-hydroxyethyl)carbamate (300 mg, 0.44 mmol) in DCM (10 mL) was added (2S)-2-methylpyrrolidine-2-carboxylic acid (86.0 mg, 0.67 mmol), sodium acetate (72.9 mg, 0.89 mmol), sodium cyanoborohydride (83.7 mg, 1.33 mmol) and stirred for 12 hr at rt. The product, (S)-1-((8-(3′-(5-(((tert-butoxycarbonyl)(2-hydroxyethyl)amino)methyl)-6-methoxypyridin-2-yl)-2,2′-dichloro-[1,1′-biphenyl]-3-yl)-4-oxo-4H-pyrido[1,2-a]pyrimidin-3-yl)methyl)-2-methylpyrrolidine-2-carboxylic acid (350 mg, crude) was obtained as a crude yellow DCM solution (10 mL), and was used into the next step without further purification.

### (S)-1-((8-(2,2′-Dichloro-3′-(5-(((2-hydroxyethyl)amino)methyl)-6-methoxypyridin-2-yl)-[1,1′-biphenyl]-3-yl)-4-oxo-4H-pyrido[1,2-a]pyrimidin-3-yl)methyl)-2-methylpyrrolidine-2-carboxylic acid

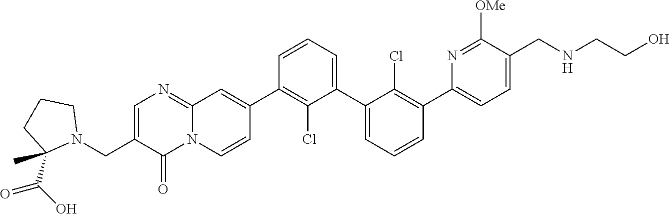 [SMILES: COc1nc(-c2cccc(-c3cccc(-c4ccn5c(=O)c(CN6CCC[C@@]6(C)C(=O)O)cnc5c4)c3Cl)c2Cl)ccc1CNCCO]

To a solution of (S)-1-((8-(3′-(5-(((tert-butoxycarbonyl)(2-hydroxyethyl)amino) methyl)-6-methoxypyridin-2-yl)-2,2′-dichloro-[1,1′-biphenyl]-3-yl)-4-oxo-4H-pyrido [1,2-a]pyrimidin-3-yl)methyl)-2-methylpyrrolidine-2-carboxylic acid (350 mg, 0.44 mmol) in DCM (10 mL) was added 2,2,2-trifluoroacetic acid (14.58 mL, 197 mmol) and stirred for 2.5 hr at r.t. The mixture was concentrated in vacuum directly and purified by reverse phase HPLC to afford (S)-1-((8-(2,2′-dichloro-3′-(5-(((2-hydroxyethyl)amino)methyl)-6-methoxypyridin-2-yl)-[1,1′-biphenyl]-3-yl)-4-oxo-4H-pyrido[1,2-a]pyrimidin-3-yl) methyl)-2-methylpyrrolidine-2-carboxylic acid as formic acid salt as a white solid (89.8 mg, 26% yield). m/z: 688 [M+H]⁺ (method A)¹H NMR (400 MHz, Methanol-d₄): δ 9.21 (d, J=7.2 Hz, 1H), 8.58 (s, 1H), 7.90-7.87 (m, 2H), 7.71-7.63 (m, 6H), 7.46 (dd, J=7.6 Hz, J=1.2 Hz, 1H), 7.37 (d, J=7.6 Hz, 1H), 4.51 (d, J=12.8 Hz, 1H), 4.29-4.22 (m, 3H), 4.11 (s, 3H), 3.87-3.85 (m, 2H), 3.61 (t, J=9.2 Hz, 1H), 3.33-3.30 (m, 1H), 3.19 (t, J=5.2 Hz, 2H), 2.46 (t, J=8.4 Hz, 1H), 2.14-2.06 (m, 2H), 1.92-1.89 (m, 1H), 1.72 (s, 3H).

### Example 172: (S)-1-((8-(2,2′-Dichloro-3′-(5-(((2-hydroxyethyl)amino)methyl)-6-methoxypyridin-2-yl)-[1,1′-biphenyl]-3-yl)-4-oxo-4H-pyrido[1,2-a]pyrimidin-3-yl)methyl)pyrrolidine-3-carboxylic acid

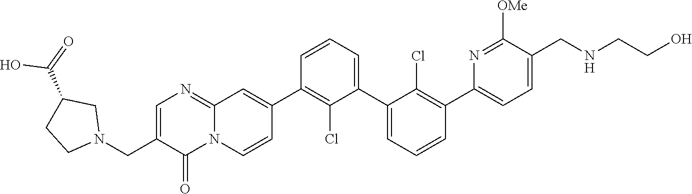 [SMILES: COc1nc(-c2cccc(-c3cccc(-c4ccn5c(=O)c(CN6CC[C@H](C(=O)O)C6)cnc5c4)c3Cl)c2Cl)ccc1CNCCO]

This compound was prepared in a similar fashion to Example 168, using (S)-pyrrolidine-3-carboxylic acid and tert-butyl ((6-(2,2′-dichloro-3′-(3-formyl-4-oxo-4H-pyrido[1,2-a]pyrimi din-8-yl)-[1,1′-biphenyl]-3-yl)-2-methoxypyridin-3-yl)methyl)(2-hydroxyethyl)carbamate. MS: m/z found 674 [M+H]⁺, (Method A). ¹H NMR (400 MHz, Methanol-d₄): δ 9.20 (d, J=7.6 Hz, 1H), 8.54 (s, 1H), 7.86 (s, 1H), 7.78 (d, J=7.6 Hz, 1H), 7.69-7.61 (m, 4H), 7.55-7.52 (m, 2H), 7.43 (d, J=7.2 Hz, 1H), 7.29 (d, J=7.2 Hz, 1H), 4.33 (s, 2H), 4.06-4.01 (m, 4H), 3.97 (s, 2H), 3.75 (t, J=5.6 Hz, 2H), 3.50-3.37 (m, 3H), 3.12-3.07 (m, 1H), 2.88 (t, J=4.2 Hz, 2H), 2.32-2.23 (m, 2H).

### Example 191: ((8-(2,2′-Dichloro-3′-(5-(((2-hydroxyethyl)amino)methyl)-6-methoxypyridin-2-yl)-[1,1′-biphenyl]-3-yl)-4-oxo-4H-pyrido[1,2-a]pyrimidin-3-yl)methyl)-L-serine

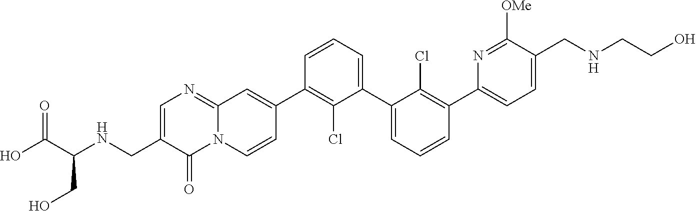 [SMILES: COc1nc(-c2cccc(-c3cccc(-c4ccn5c(=O)c(CN[C@@H](CO)C(=O)O)cnc5c4)c3Cl)c2Cl)ccc1CNCCO]

This compound was prepared in a similar fashion to Example 168, using (S)-2-amino-3-hydroxypropanoic acid and tert-butyl ((6-(2,2′-dichloro-3′-(3-formyl-4-oxo-4H-pyrido[1,2-a]pyrimidin-8-yl)-[1,1′-biphenyl]-3-yl)-2-methoxypyridin-3-yl)methyl)(2-hydroxyethyl)carbamate. MS: m/z found 664 [M+H]⁺, (Method A). ¹H NMR (400 MHz, Methanol-d₄): δ 9.17 (d, J=7.6 Hz, 1H), 8.52 (s, 1H), 7.87 (t, J=7.2 Hz, 2H), 7.68-7.61 (m, 4H), 7.54-7.51 (m, 2H), 7.44 (d, J=2.0 Hz, 1H), 7.36 (d, J=7.2 Hz, 1H), 4.35 (d, J=36 Hz, 4H), 4.11-4.07 (m, 4H), 3.98-3.94 (m, 1H), 3.86-3.83 (m, 3H), 3.21-3.19 (m, 2H).

### Example 195: 8-(2,2′-Dichloro-3′-(5-((((S)-2-hydroxypropyl)amino)methyl)-6-methoxypyridin-2-yl)-[1,1′-biphenyl]-3-yl)-3-((((R)-2-hydroxypropyl)amino)methyl)-4H-pyrido[1,2-a]pyrimidin-4-one

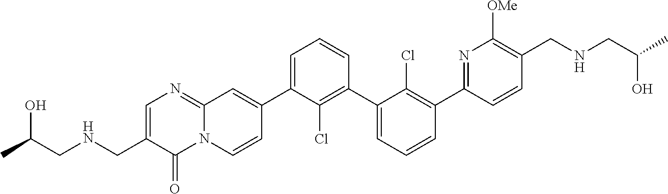 [SMILES: COc1nc(-c2cccc(-c3cccc(-c4ccn5c(=O)c(CNC[C@@H](C)O)cnc5c4)c3Cl)c2Cl)ccc1CNC[C@H](C)O]

This compound was prepared in a similar fashion to Example 168, using (2S)-1-aminopropan-2-ol and 6-(3-bromo-2-chloro-phenyl)-2-methoxy-pyridine-3-carbaldehyde. MS: m/z found 648 [M+H]⁺, (Method C). ¹H NMR (400 MHz, Methanol-d₄): δ 9.17 (d, J=7.2 Hz, 1H), 8.49 (s, 1H), 7.84 (m, 2H), 7.65 (m, 4H), 7.63 (m, 2H), 7.43 (m, 1H), 7.33 (d, J=7.6 Hz, 1H), 4.19 (m, 4H), 4.04 (m, 5H), 3.04 (m, 2H), 2.84 (m, 2H), 1.22 (m, 6H).

### Example 196: 8-(2,2′-Dichloro-3′-(5-((((R)-2-hydroxypropyl)amino)methyl)-6-methoxypyridin-2-yl)-[1,1′-biphenyl]-3-yl)-3-((((S)-2-hydroxypropyl)amino)methyl)-4H-pyrido[1,2-a]pyrimidin-4-one

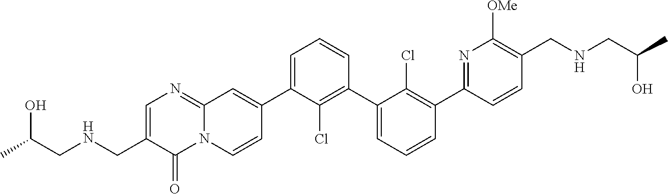 [SMILES: COc1nc(-c2cccc(-c3cccc(-c4ccn5c(=O)c(CNC[C@H](C)O)cnc5c4)c3Cl)c2Cl)ccc1CNC[C@@H](C)O]

This compound was prepared in a similar fashion to Example 168, using (2R)-1-aminopropan-2-ol and 6-(3-bromo-2-chloro-phenyl)-2-methoxy-pyridine-3-carbaldehyde. MS: m/z found 648 [M+H]⁺, (Method C). ¹H NMR (400 MHz, Methanol-d₄): δ9.18 (d, J=7.2 Hz, 1H), 8.51 (s, 1H), 8.86 (m, 2H), 7.68 (m, 1H), 7.62 (m, 3H), 7.54 (m, 2H), 7.43 (m, 1H), 7.35 (d, J=7.6 Hz, 1H), 4.27 (m, 4H), 4.07 (m, 5H), 3.11 (m, 2H), 2.89 (m, 2H), 1.24 (m, 6H).

### Example 188: 1-((8-(2,2′-Dichloro-3′-(5-(((2-hydroxyethyl)amino)methyl)-6-methoxypyridin-2-yl)-[1,1′-biphenyl]-3-yl)-4-oxo-4H-pyrido[1,2-a]pyrimidin-3-yl)methyl)azetidine-3-carboxylic acid

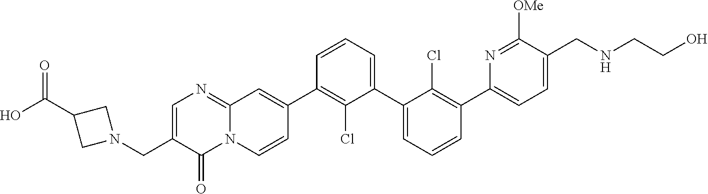 [SMILES: COc1nc(-c2cccc(-c3cccc(-c4ccn5c(=O)c(CN6CC(C(=O)O)C6)cnc5c4)c3Cl)c2Cl)ccc1CNCCO]

This compound was prepared in a similar fashion to Example 168, but using azetidine-3-carboxylic acid and tert-butyl ((6-(2,2′-dichloro-3′-(3-formyl-4-oxo-4H-pyrido[1,2-a]pyrimidin-8-yl)-[1,1′-biphenyl]-3-yl)-2-methoxypyridin-3-yl)methyl)(2-hydroxyethyl)carbamate in the final reductive amination. MS: m/z found 660 [M+H]⁺, (Method A). ¹H NMR (400 MHz, Methanol-d₄): δ 9.19-9.17 (m, 1H), 8.50 (s, 1H), 7.84-7.80 (m, 2H), 7.69-7.53 (m, 6H), 7.44-7.30 (m, 2H), 4.27 (d, J=8.4 Hz, 2H), 4.18-4.05 (m, 8H), 3.79-3.77 (m, 2H), 3.42-3.33 (m, 2H), 3.96-3.95 (m, 1H), 2.68 (s, 1H).

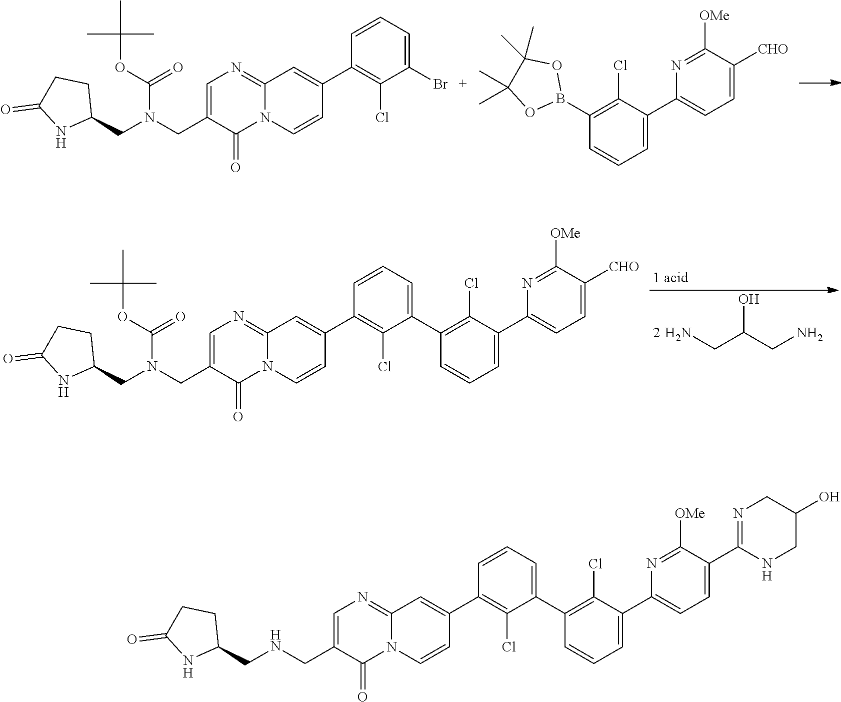

### Example 178: 8-(2,2′-Dichloro-3′-(5-(5-hydroxy-1,4,5,6-tetrahydropyrimidin-2-yl)-6-methoxypyridin-2-yl)-[1,1′-biphenyl]-3-yl)-3-(((((S)-5-oxopyrrolidin-2-yl)methyl)amino)methyl)-4H-pyrido[1,2-a]pyrimidin-4-one

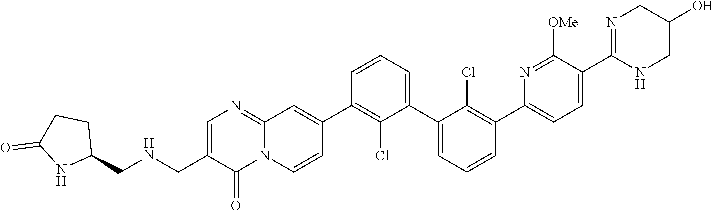 [SMILES: COc1nc(-c2cccc(-c3cccc(-c4ccn5c(=O)c(CNC[C@@H]6CCC(=O)N6)cnc5c4)c3Cl)c2Cl)ccc1C1=NCC(O)CN1]

### (S)-tert-Butyl((8-(2,2′-dichloro-3′-(5-formyl-6-methoxypyridin-2-yl)-[1,1′-biphenyl]-3-yl)-4-oxo-4H-pyrido[1,2-a]pyrimidin-3-yl)methyl)((5-oxopyrrolidin-2-yl)methyl)carbamate

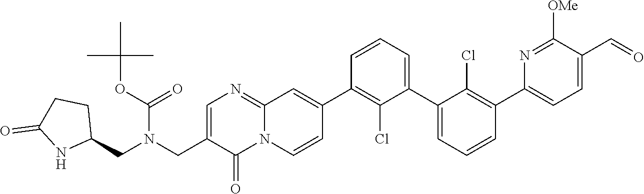 [SMILES: COc1nc(-c2cccc(-c3cccc(-c4ccn5c(=O)c(CN(C[C@@H]6CCC(=O)N6)C(=O)OC(C)(C)C)cnc5c4)c3Cl)c2Cl)ccc1C=O]

To a solution of tert-butyl (S)-tert-butyl ((8-(3-bromo-2-chlorophenyl)-4-oxo-4H-pyrido[1,2-a]pyrimidin-3-yl)methyl)((5-oxopyrrolidin-2-yl)methyl)carbamate (3 g, 5.34 mmol), 6-(2-chloro-3-(4,4,5,5-tetramethyl-1,3,2-dioxaborolan-2-yl)phenyl)-2-methoxynicotinaldehyde (2.99 g, 8.01 mmol) in dioxane/H₂O mixture (5:1, 60 mL) was added potassium phosphate (3.4 g, 16 mmol), Dichloro[1,1′-bis(di-1-propylphosphino)ferrocene]palladium(II) (0.35 g, 0.53 mmol). The reaction was stirred at 110° C. for 0.5 h under N₂, then concentrated down. The residue was purified by normal phase SiO₂ chromatography (0-80% EtOAc/petroleum ether) to afford tert-butyl (S)-tert-butyl((8-(2,2′-dichloro-3′-(5-formyl-6-methoxypyridin-2-yl)-[1,1′-biphenyl]-3-yl)-4-oxo-4H-pyrido[1,2-a]pyrimidin-3-yl)methyl)((5-oxopyrrolidin-2-yl)methyl)carbamate (3.2 g, 82% yield) as a brown solid. ¹H NMR (400 MHz, DMSO-d₆): δ 10.28 (s, 1H), 9.02 (d, J=7.9 Hz, 1H), 8.23-8.19 (m, 2H), 7.76-7.75 (m, 2H), 7.62-7.46 (m, 7H), 4.42-2.32 (m, 2H), 4.04 (s, 3H), 3.83-3.78 (m, 1H), 3.36 (s, 2H), 2.23-2.04 (m, 4H), 1.38 (s, 9H).

### (S)-6-(2,2′-Dichloro-3′-(4-oxo-3-((((5-oxopyrrolidin-2-yl)methyl)amino)methyl)-4H-pyrido[1,2-a]pyrimidin-8-yl)-[1,1′-biphenyl]-3-yl)-2-methoxynicotinaldehyde

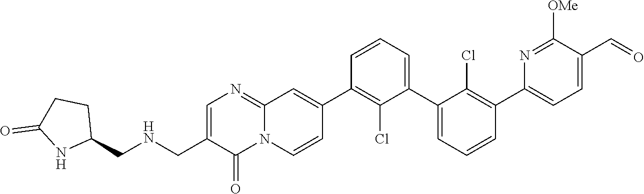 [SMILES: COc1nc(-c2cccc(-c3cccc(-c4ccn5c(=O)c(CNC[C@@H]6CCC(=O)N6)cnc5c4)c3Cl)c2Cl)ccc1C=O]

A solution of tert-butyl (S)-tert-butyl ((8-(2,2′-dichloro-3′-(5-formyl-6-methoxypyridin-2-yl)-[1,1′-biphenyl]-3-yl)-4-oxo-4H-pyrido[1,2-a]pyrimidin-3-yl)methyl)((5-oxopyrrolidin-2-yl)methyl)carbamate (300 mg, 0.41 mmol), trifluoroacetic acid (1.5 mL, 20.3 mmol) in DCM (15 mL) was stirred at rt. for 0.5 h under N₂. The mixture was concentrated to afford (S)-6-(2,2′-dichloro-3′-(4-oxo-3-((((5-oxopyrrolidin-2-yl)methyl)amino)methyl)-4H-pyrido[1,2-a]pyrimidin-8-yl)-[1,1′-biphenyl]-3-yl)-2-methoxynicotinaldehyde (300 mg, crude, m/z: 628 [M+H]⁺ observed, Method AD) as a brown oil which was used for next step without further purification.

### 8-(2,2′-Dichloro-3′-(5-(5-hydroxy-1,4,5,6-tetrahydropyrimidin-2-yl)-6-methoxypyridin-2-yl)-[1,1′-biphenyl]-3-yl)-3-(((((S)-5-oxopyrrolidin-2-yl)methyl)amino)methyl)-4H-pyrido[1,2-a]pyrimidin-4-one

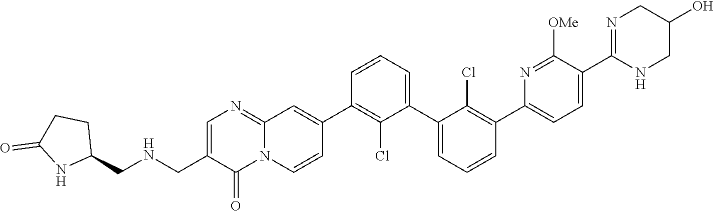 [SMILES: COc1nc(-c2cccc(-c3cccc(-c4ccn5c(=O)c(CNC[C@@H]6CCC(=O)N6)cnc5c4)c3Cl)c2Cl)ccc1C1=NCC(O)CN1]

A mixture of (S)-6-(2,2′-dichloro-3′-(4-oxo-3-((((5-oxopyrrolidin-2-yl)methyl)amino)methyl)-4H-pyrido[1,2-a]pyrimidin-8-yl)-[1,1′-biphenyl]-3-yl)-2-methoxynicotinaldehyde (300 mg, 0.48 mmol), 1,3-diaminopropan-2-ol (43 mg, 0.48 mmol), potassium carbonate (660 mg, 4.77 mmol), iodine (727 mg, 2.86 mmol) in DMA (3 mL) was stirred at r.t for 12 h under N₂. The reaction was filtered and concentrated, and the residue was purified by reverse phase HPLC to afford 8-(2,2′-dichloro-3′-(5-(5-hydroxy-1,4,5,6-tetrahydropyrimidin-2-yl)-6-methoxypyridin-2-yl)-[1,1′-biphenyl]-3-yl)-3-(((((S)-5-oxopyrrolidin-2-yl)methyl)amino)methyl)-4H-pyrido[1,2-a]pyrimidin-4-one as a formic acid salt (33.7 mg, 9% yield, m/z: 698 [M+H]⁺ observed, Method A) as a white solid. ¹H NMR (400 MHz, CD₃OD): δ9.18 (d, J=7.2 Hz, 1H), 8.49-8.44 (m, 2H), 8.09 (d, J=7.6 Hz, 1H), 7.83 (d, J=0.8 Hz, 1H), 7.75-7.73 (m, 1H), 7.66-7.51 (m, 7H), 4.43-4.42 (m, 1H), 4.12 (s, 3H), 4.09 (d, J=4.4 Hz, 2H), 3.98-3.95 (m, 1H), 3.73-3.69 (m, 2H), 3.57-3.53 (m, 2H), 3.02-2.93 (m, 2H), 2.41-2.33 (m, 3H), 1.91-1.87 (m, 1H).

### Example 170: 8-(2,2′-Dichloro-3′-(5-(((R)-3-hydroxypyrrolidin-1-yl)methyl)-6-methoxypyridin-2-yl)-[1,1′-biphenyl]-3-yl)-3-(((((S)-5-oxopyrrolidin-2-yl)methyl)amino)methyl)-4H-pyrido[1,2-a]pyrimidin-4-one

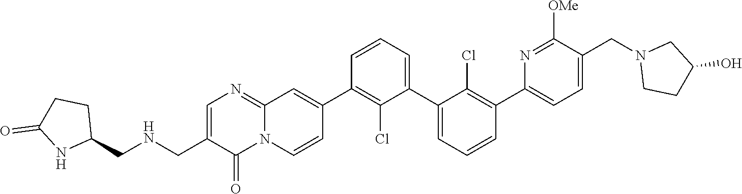 [SMILES: COc1nc(-c2cccc(-c3cccc(-c4ccn5c(=O)c(CNC[C@@H]6CCC(=O)N6)cnc5c4)c3Cl)c2Cl)ccc1CN1CC[C@@H](O)C1]

This compound was prepared in a similar fashion to Example 178, using (R)-pyrrolidin-3-ol and tert-butyl (S)-((8-(2,2′-dichloro-3′-(5-formyl-6-methoxypyridin-2-yl)-[1,1′-biphenyl]-3-yl)-4-oxo-4H-pyrido[1,2-a]pyrimidin-3-yl)methyl)((5-oxopyrrolidin-2-yl)methyl)carbamate. MS: m/z found 699 [M+H]⁺, (Method C). ¹H NMR (400 MHz, CD₃OD): δ9.17 (d, J=7.2 Hz, 1H), 8.46 (m, 2H), 7.92 (d, J=7.6 Hz, 1H), 7.70 (s, 1H), 7.69-7.65 (m, 1H), 7.64-7.54 (m, 5H), 7.46 (d, J=1.2 Hz, 1H), 7.38 (d, J=7.2 Hz, 1H), 4.57 (s, 1H), 4.43-4.35 (m, 2H), 4.10-4.04 (m, 5H), 3.91 (s, 1H), 3.52-3.27 (m, 4H), 2.94-2.90 (m, 1H), 2.38-2.33 (m, 4H), 2.07-2.06 (m, 1H), 1.88-1.87 (m, 1H).

### Example 180: 2-(6-(2,2′-Dichloro-3′-(4-oxo-3-(((((S)-5-oxopyrrolidin-2-yl)methyl)amino) methyl)-4H-pyrido[1,2-a]pyrimidin-8-yl)-[1,1′-biphenyl]-3-yl)-2-methoxypyridin-3-yl)-3,4,5,6-tetrahydropyrimidine-4-carboxylic acid

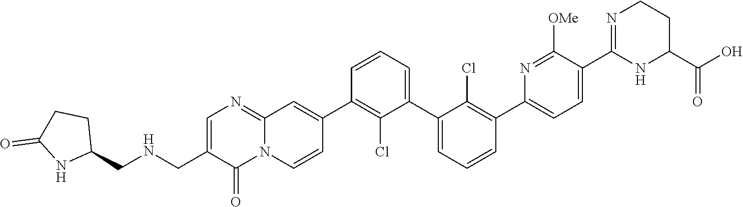 [SMILES: COc1nc(-c2cccc(-c3cccc(-c4ccn5c(=O)c(CNC[C@@H]6CCC(=O)N6)cnc5c4)c3Cl)c2Cl)ccc1C1=NCCC(C(=O)O)N1]

This compound was prepared in a similar fashion to Example 178, but using 2,4-diaminobutanoic acid HCl and (S)-6-(2,2′-dichloro-3′-(4-oxo-3-((((5-oxopyrrolidin-2-yl)methyl)amino) methyl)-4H-pyrido[1,2-a]pyrimidin-8-yl)-[1,1′-biphenyl]-3-yl)-2-methoxynicotinaldehyde TFA. MS: m/z found 726 [M+H]⁺, (Method A). ¹H NMR (400 MHz, CD₃OD): δ9.19 (d, J=7.6 Hz, 1H), 8.49 (s, 1H), 8.41 (bs, 1H), 8.18 (d, J=7.6 Hz, 1H), 7.84 (d, J=1.6 Hz, 1H), 7.74 (dd, J=1.6 Hz, J=7.6 Hz, 1H), 7.66-7.50 (m, 7H), 4.20-3.97 (m, 7H), 3.67-3.58 (m, 2H), 3.02-2.96 (m, 2H), 2.41-2.28 (m, 5H), 1.90-1.87 (m, 1H).

### Example 198: 8-(2,2′-Dichloro-3′-(5-((((R)-2-hydroxypropyl)amino)methyl)-6-methoxypyridin-2-yl)-[1,1′-biphenyl]-3-yl)-3-(((((S)-5-oxopyrrolidin-2-yl)methyl)amino)methyl)-4H-pyrido[1,2-a]pyrimidin-4-one

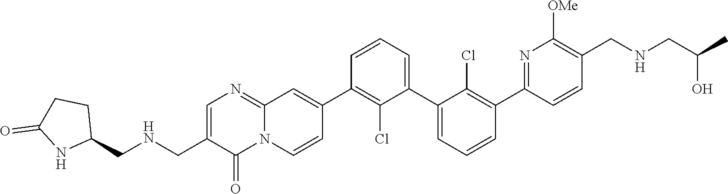 [SMILES: COc1nc(-c2cccc(-c3cccc(-c4ccn5c(=O)c(CNC[C@@H]6CCC(=O)N6)cnc5c4)c3Cl)c2Cl)ccc1CNC[C@@H](C)O]

This compound was prepared in a similar fashion to Example 178, but using (5S)-5-(aminomethyl)pyrrolidin-2-one and (S)-6-(2,2′-dichloro-3′-(4-oxo-3-((((5-oxopyrrolidin-2-yl)methyl)amino) methyl)-4H-pyrido[1,2-a]pyrimidin-8-yl)-[1,1′-biphenyl]-3-yl)-2-methoxynicotinaldehyde TFA. MS: m/z found 687 [M+H]⁺, (Method C). ¹H NMR (400 MHz, CD₃OD): δ9.18 (d, J=7.2 Hz, 1H), 8.49 (s, 1H), 7.85 (m, 2H), 7.65 (m, 4H), 7.53 (m, 2H), 7.44 (d, J=6.0 Hz 1H), 7.36 (d, J=8.0 Hz, 1H), 4.30 (s, 2H), 4.19 (s, 2H), 4.09 (s, 4H), 3.99 (s, 1H), 3.12 (m, 3H), 2.91 (m, 1H), 2.39 (m, 3H), 1.88 (d, J=7.6 Hz, 1H), 1.25 (d, J=6.4 Hz, 3H).

### Example 347: 8-(2,2′-Dichloro-3′-(6-methoxy-5-(((((S)-5-oxopyrrolidin-2-yl)methyl)amino)methyl)pyridin-2-yl)-[1,1′-biphenyl]-3-yl)-3-(((3R,4R)-3,4-dihydroxypyrrolidin-1-yl)methyl)-4H-pyrido[1,2-a]pyrimidin-4-one

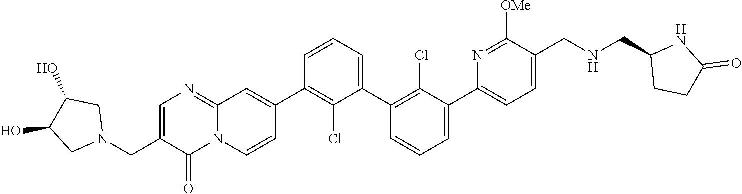 [SMILES: COc1nc(-c2cccc(-c3cccc(-c4ccn5c(=O)c(CN6C[C@@H](O)[C@H](O)C6)cnc5c4)c3Cl)c2Cl)ccc1CNC[C@@H]1CCC(=O)N1]

This compound was prepared in a similar fashion to Example 94, using (3R,4R)-pyrrolidine-3,4-diol and tert-butyl (S)-((6-(2,2′-dichloro-3′-(3-formyl-4-oxo-4H-pyrido[1,2-a]pyrimidin-8-yl)-[1,1′-biphenyl]-3-yl)-2-methoxypyridin-3-yl)methyl)((5-oxopyrrolidin-2-yl)methyl)carbamate. MS: m/z found 715 [M+H]⁺; ¹H NMR (400 MHz, Methanol-d₄) δ 9.00 (t, J=8.2, 4.4 Hz, 1H), 8.43 (s, 2H), 8.34 (d, J=2.6 Hz, 1H), 8.01-7.92 (m, 2H), 7.61-7.33 (m, 5H), 7.33-7.22 (m, 2H), 7.06 (dd, J=7.4, 3.0 Hz, 1H), 4.66 (s, 1H), 4.28-4.15 (m, 4H), 3.88 (d, J=1.5 Hz, 3H), 3.84-3.69 (m, 2H), 3.59 (d, J=12.1 Hz, 2H), 3.29 (d, J=14.8 Hz, 2H), 2.94-2.80 (m, 3H), 2.64-2.48 (m, 1H), 2.43-2.28 (m, 1H), 2.26-2.15 (m, 1H).

### Example 346: (S)-8-(2,2′-Dichloro-3′-(6-methoxy-5-((((5-oxopyrrolidin-2-yl)methyl) amino)methyl)pyridin-2-yl)-[1,1′-biphenyl]-3-yl)-3-((6-hydroxy-2-azaspiro[3.3]heptan-2-yl)methyl)-4H-pyrido[1,2-a]pyrimidin-4-one

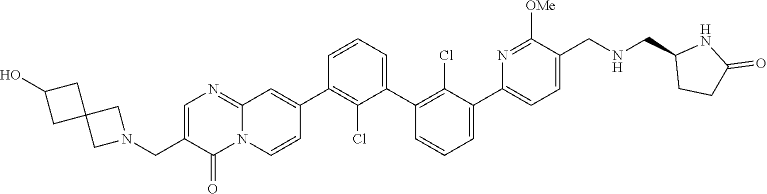 [SMILES: COc1nc(-c2cccc(-c3cccc(-c4ccn5c(=O)c(CN6CC7(CC(O)C7)C6)cnc5c4)c3Cl)c2Cl)ccc1CNC[C@@H]1CCC(=O)N1]

This compound was prepared in a similar fashion to Example 94, using 2-azaspiro[3.3]heptan-6-ol and tert-butyl (S)-((6-(2,2′-dichloro-3′-(3-formyl-4-oxo-4H-pyrido[1,2-a]pyrimidin-8-yl)-[1,1′-biphenyl]-3-yl)-2-methoxypyridin-3-yl)methyl)((5-oxopyrrolidin-2-yl)methyl)carbamate. MS: m/z found 725 [M+H]⁺; ¹H NMR (400 MHz, Methanol-d₄) δ 8.97 (dd, J=8.1, 3.5 Hz, 1H), 8.44 (s, 2H), 8.27 (d, J=2.5 Hz, 1H), 7.95 (s, 2H), 7.57 (dd, J=7.4, 2.9 Hz, 1H), 7.54-7.33 (m, 4H), 7.32-7.22 (m, 2H), 7.06 (dd, J=7.4, 4.1 Hz, 1H), 4.60 (s, 2H), 4.15-4.05 (m, 2H), 4.02 (d, J=12.9 Hz, 3H), 3.88 (s, 3H), 3.76 (d, 2H), 2.92-2.78 (m, 3H), 2.64-2.48 (m, 3H), 2.40-2.29 (m, 1H), 2.25-2.17 (m, 1H), 2.13-2.03 (m, 1H).

### Example 345: (S)-8-(2,2′-Dichloro-3′-(6-methoxy-5-((((5-oxopyrrolidin-2-yl)methyl)amino)methyl)pyridin-2-yl)-[1,1′-biphenyl]-3-yl)-3-(((2-methoxy-2-methylpropyl)amino)methyl)-4H-pyrido[1,2-a]pyrimidin-4-one

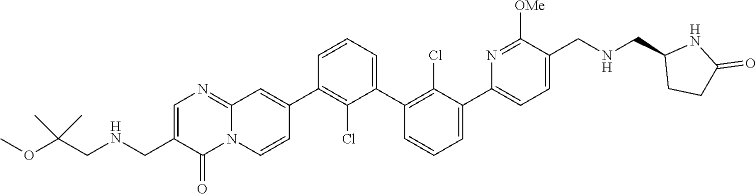 [SMILES: COc1nc(-c2cccc(-c3cccc(-c4ccn5c(=O)c(CNCC(C)(C)OC)cnc5c4)c3Cl)c2Cl)ccc1CNC[C@@H]1CCC(=O)N1]

This compound was prepared in a similar fashion to Example 94, using 2-methoxy-2-methylpropan-1-amine and tert-butyl (S)-((6-(2,2′-dichloro-3′-(3-formyl-4-oxo-4H-pyrido[1,2-a]pyrimidin-8-yl)-[1,1′-biphenyl]-3-yl)-2-methoxypyridin-3-yl)methyl)((5-oxopyrrolidin-2-yl)methyl)carbamate. MS: m/z found 715 [M+H]⁺; ¹H NMR (400 MHz, Methanol-d₄) δ 9.14 (d, J=7.4, 0.7 Hz, 1H), 8.47 (s, 1H), 8.39 (s, 2H), 7.85-7.74 (m, 2H), 7.71-7.53 (m, 4H), 7.54-7.44 (m, 2H), 7.37 (d, J=7.6, 1.7 Hz, 1H), 7.25 (d, J=7.5 Hz, 1H), 4.26 (s, 2H), 4.09-3.96 (m, 5H), 3.93-3.88 (m, 1H), 3.22 (s, 2H), 3.09 (s, 2H), 2.99-2.84 (m, 2H), 2.33-2.25 (m, 2H), 1.89-1.75 (m, 1H), 1.24 (s, 6H).

### Example 301: (S)-8-(2,2′-Dichloro-3′-(6-methoxy-5-((((5-oxopyrrolidin-2-yl)methyl) amino)methyl)pyridin-2-yl)-[1,1′-biphenyl]-3-yl)-3-((((1-(hydroxymethyl) cyclopropyl)methyl)amino)methyl)-4H-pyrido[1,2-a]pyrimidin-4-one

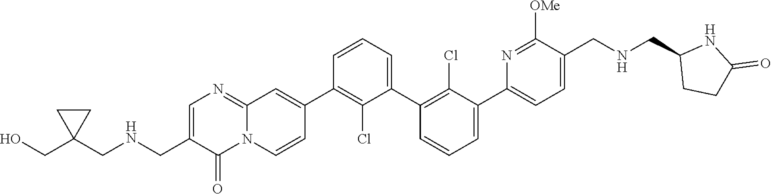 [SMILES: COc1nc(-c2cccc(-c3cccc(-c4ccn5c(=O)c(CNCC6(CO)CC6)cnc5c4)c3Cl)c2Cl)ccc1CNC[C@@H]1CCC(=O)N1]

This compound was prepared in a similar fashion to Example 94, using (1-(aminomethyl) cyclopropyl)methanol and tert-butyl (S)-((6-(2,2′-dichloro-3′-(3-formyl-4-oxo-4H-pyrido[1,2-a]pyrimidin-8-yl)-[1,1′-biphenyl]-3-yl)-2-methoxypyridin-3-yl)methyl)((5-oxopyrrolidin-2-yl)methyl)carbamate. MS: m/z found 713 [M+H]⁺; ¹H NMR (400 MHz, Methanol-d₄) δ 9.13 (d, 1H), 8.47-8.38 (m, 3H), 7.84-7.72 (m, 2H), 7.65-7.57 (m, 2H), 7.60-7.55 (m, 1H), 7.50-7.45 (m, 2H), 7.37 (dd, J=7.6, 1.7 Hz, 1H), 7.24 (d, J=7.4 Hz, 1H), 4.24 (s, 2H), 3.98 (d, J=12.1 Hz, 5H), 3.88 (s, 1H), 3.51 (s, 2H), 3.16 (s, 2H), 2.93-2.78 (m, 2H), 2.39-2.21 (m, 3H), 1.87-1.74 (m, 1H), 0.71-0.58 (m, 4H).

### Example 300: (S)-8-(2,2′-Dichloro-3′-(6-methoxy-5-((((5-oxopyrrolidin-2-yl)methyl)amino)methyl)pyridin-2-yl)-[1,1′-biphenyl]-3-yl)-3-(((2,2-difluoro-3-hydroxypropyl)amino)methyl)-4H-pyrido[1,2-a]pyrimidin-4-one

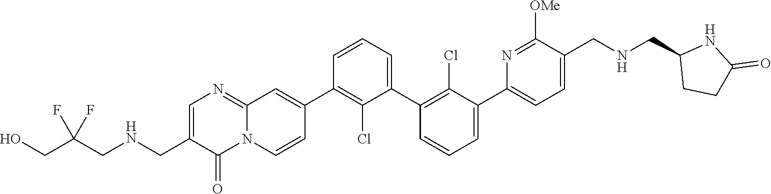 [SMILES: COc1nc(-c2cccc(-c3cccc(-c4ccn5c(=O)c(CNCC(F)(F)CO)cnc5c4)c3Cl)c2Cl)ccc1CNC[C@@H]1CCC(=O)N1]

This compound was prepared in a similar fashion to Example 94, using 3-amino-2,2-difluoropropan-1-ol and tert-butyl (S)-((6-(2,2′-dichloro-3′-(3-formyl-4-oxo-4H-pyrido[1,2-a]pyrimidin-8-yl)-[1,1′-biphenyl]-3-yl)-2-methoxypyridin-3-yl)methyl)((5-oxopyrrolidin-2-yl)methyl)carbamate. MS: m/z found 723 [M+H]⁺; ¹H NMR (400 MHz, Methanol-d₄) δ 9.09 (d, J=7.4, 0.7 Hz, 1H), 8.43-8.35 (m, 2H), 7.78-7.70 (m, 2H), 7.66-7.51 (m, 3H), 7.53-7.43 (m, 3H), 7.37 (d, J=7.5, 1.8, 0.6 Hz, 1H), 7.25 (d, J=7.5 Hz, 1H), 4.03-3.91 (m, 5H), 3.88 (s, 3H), 3.73 (t, J=13.1 Hz, 2H), 3.06 (t, J=14.6 Hz, 2H), 2.89-2.75 (m, 2H), 2.39-2.21 (m, 3H), 1.87-1.74 (m, 1H).

### Example 274: 3-(((2-Cyclopropyl-2-hydroxyethyl)amino)methyl)-8-(2,2′-dichloro-3′-(6-methoxy-5-(((((S)-5-oxopyrrolidin-2-yl)methyl)amino)methyl)pyridin-2-yl)-[1,1′-biphenyl]-3-yl)-4H-pyrido[1,2-a]pyrimidin-4-one

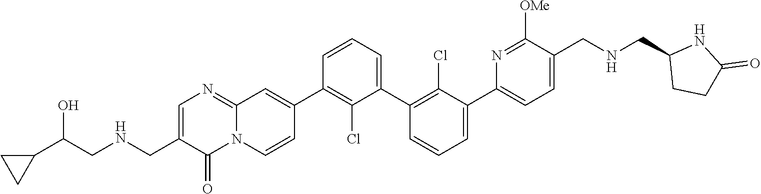 [SMILES: COc1nc(-c2cccc(-c3cccc(-c4ccn5c(=O)c(CNCC(O)C6CC6)cnc5c4)c3Cl)c2Cl)ccc1CNC[C@@H]1CCC(=O)N1]

This compound was prepared in a similar fashion to Example 94, using 2-amino-1-cyclopropylethan-1-ol and tert-butyl (S)-((6-(2,2′-dichloro-3′-(3-formyl-4-oxo-4H-pyrido[1,2-a]pyrimidin-8-yl)-[1,1′-biphenyl]-3-yl)-2-methoxypyridin-3-yl)methyl)((5-oxopyrrolidin-2-yl)methyl)carbamate. MS: m/z found 713 [M+H]⁺; ¹H NMR (400 MHz, Methanol-d₄) δ 9.13 (d, J=7.4, 3.0 Hz, 1H), 8.46 (s, 2H), 7.84-7.75 (m, 1H), 7.73 (d, J=7.5 Hz, 1H), 7.69-7.57 (m, 3H), 7.61-7.42 (m, 3H), 7.35 (d, J=7.6, 1.7 Hz, 1H), 7.22 (d, J=7.4 Hz, 1H), 4.24 (s, 2H), 3.98 (s, 2H), 3.98-3.78 (m, 4H), 3.09-2.98 (m, 1H), 2.87-2.72 (m, 3H), 2.37-2.19 (m, 3H), 1.85-1.72 (m, 1H), 1.23 (s, 1H), 0.83 (d, J=8.6 Hz, 1H), 0.58-0.43 (m, 2H), 0.42-0.33 (m, 1H), 0.29-0.20 (m, 1H).

### Example 234: (S)-8-(2,2′-Dichloro-3′-(6-methoxy-5-((((5-oxopyrrolidin-2-yl)methyl) amino)methyl)pyridin-2-yl)-[1,1′-biphenyl]-3-yl)-3-((((1-hydroxycyclopropyl) methyl)amino)methyl)-4H-pyrido[1,2-a]pyrimidin-4-one

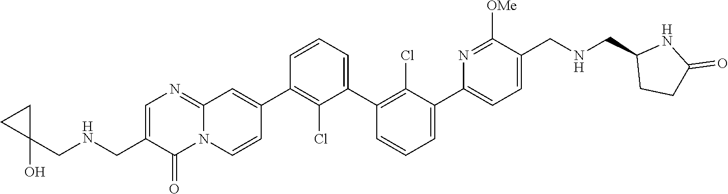 [SMILES: COc1nc(-c2cccc(-c3cccc(-c4ccn5c(=O)c(CNCC6(O)CC6)cnc5c4)c3Cl)c2Cl)ccc1CNC[C@@H]1CCC(=O)N1]

This compound was prepared in a similar fashion to Example 94, using 1-(aminomethyl)cyclopropan-1-ol and tert-butyl (S)-((6-(2,2′-dichloro-3′-(3-formyl-4-oxo-4H-pyrido[1,2-a]pyrimidin-8-yl)-[1,1′-biphenyl]-3-yl)-2-methoxypyridin-3-yl)methyl)((5-oxopyrrolidin-2-yl)methyl)carbamate. MS: m/z found 699 [M+H]⁺; H NMR (400 MHz, Methanol-d₄) δ9.14 (d, J=7.4 Hz, 1H), 8.48 (s, 1H), 8.40 (s, 2H), 7.84-7.71 (m, 2H), 7.66-7.52 (m, 4H), 7.53-7.42 (m, 2H), 7.37 (d, J=7.5, 1.7 Hz, 1H), 7.24 (d, J=7.5, 1.5 Hz, 1H), 4.31 (s, 2H), 4.00 (d, J=1.0 Hz, 5H), 3.88 (s, 1H), 3.16 (s, 2H), 2.86 (d, J=7.8 Hz, 2H), 2.36-2.18 (m, 3H), 1.86-1.74 (m, 1H), 0.91-0.81 (m, 2H), 0.73-0.65 (m, 2H).

### Example 233: (S)-8-(2,2′-Dichloro-3′-(6-methoxy-5-((((5-oxopyrrolidin-2-yl)methyl) amino)methyl)pyridin-2-yl)-[1,1′-biphenyl]-3-yl)-3-((((1-hydroxycyclobutyl) methyl)amino)methyl)-4H-pyrido[1,2-a]pyrimidin-4-one

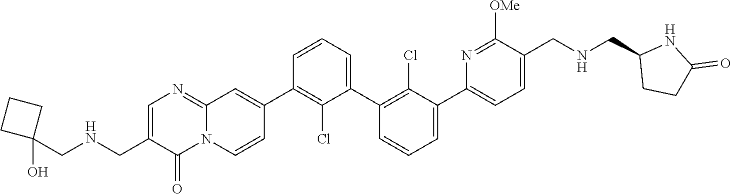 [SMILES: COc1nc(-c2cccc(-c3cccc(-c4ccn5c(=O)c(CNCC6(O)CCC6)cnc5c4)c3Cl)c2Cl)ccc1CNC[C@@H]1CCC(=O)N1]

This compound was prepared in a similar fashion to Example 94, using 1-(aminomethyl)cyclopropan-1-ol and tert-butyl (S)-((6-(2,2′-dichloro-3′-(3-formyl-4-oxo-4H-pyrido[1,2-a]pyrimidin-8-yl)-[1,1′-biphenyl]-3-yl)-2-methoxypyridin-3-yl)methyl)((5-oxopyrrolidin-2-yl)methyl)carbamate. MS: m/z found 713 [M+H]⁺; ¹H NMR (400 MHz, Methanol-d₄) δ 9.08 (d, J=7.4 Hz, 1H), 8.37 (s, 1H), 7.75-7.66 (m, 2H), 7.65-7.51 (m, 3H), 7.53-7.42 (m, 3H), 7.35 (d, J=7.6, 1.7 Hz, 1H), 7.20 (d, J=7.4 Hz, 1H), 3.97 (s, 3H), 3.86 (s, 2H), 3.85-3.72 (m, 3H), 2.73 (s, 2H), 2.69-2.57 (m, 2H), 2.34-2.18 (m, 3H), 2.12-1.93 (m, 4H), 1.81-1.67 (m, 2H), 1.52 (t, J=19.4, 9.5 Hz, 1H).

### Example 208: 8-(2′-Chloro-3′-(6-methoxy-5-(((((S)-5-oxopyrrolidin-2-yl)methyl)amino) methyl)pyridin-2-yl)-2-(trifluoromethyl)-[1,1′-biphenyl]-3-yl)-3-(((((S)-5-oxopyrrolidin-2-yl)methyl)amino)methyl)-4H-pyrido[1,2-a]pyrimidin-4-one

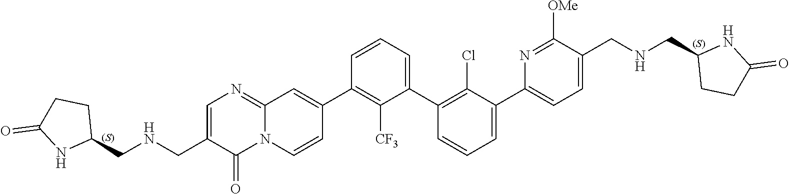

This compound was prepared in a similar fashion to Example 140, but using 1,3-dibromo-2-(trifluoromethyl)benzene and (3-bromo-4-oxo-4H-pyrido[1,2-a]pyrimidin-8-yl)boronic acid to couple first then combining with 6-(2-chloro-3-(4,4,5,5-tetramethyl-1,3,2-dioxaborolan-2-yl)phenyl)-2-methoxynicotinaldehyde. MS: m/z found 760 [M+H. ¹H NMR (400 MHz, Methanol-d₄) δ 9.16 (d, J=7.4, 0.8 Hz, 1H), 8.46 (s, 1H), 8.33 (s, 1H), 7.85-7.77 (m, 2H), 7.73-7.65 (m, 2H), 7.60-7.49 (m, 3H), 7.43 (d, J=7.6, 1.8 Hz, 1H), 7.30 (d, J=7.5, 4.0 Hz, 1H), 4.15-3.97 (m, 6H), 3.96-3.91 (m, 2H), 3.04-2.86 (m, 4H), 2.43-2.25 (m, 7H), 1.92-1.79 (m, 2H).

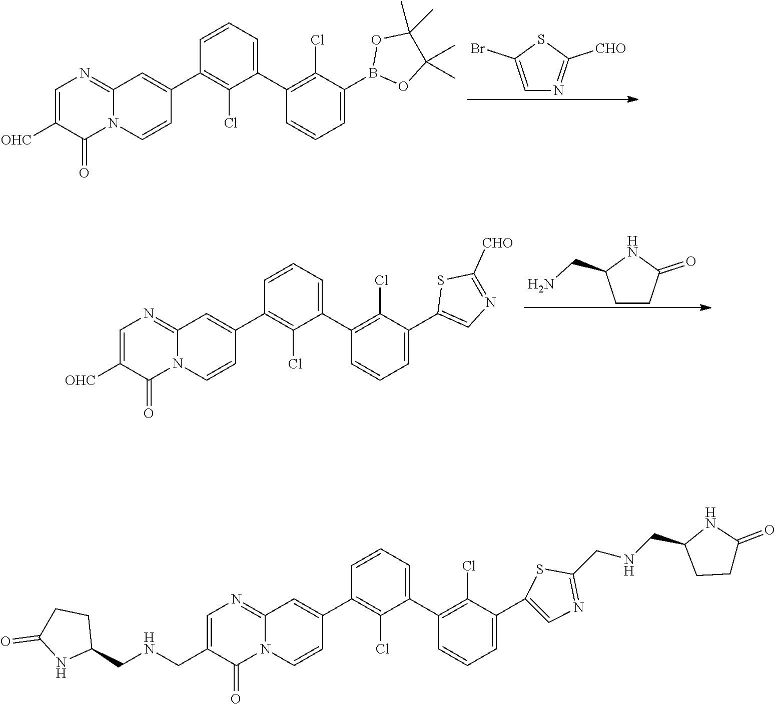

### Example 442: 8-(2,2′-dichloro-3′-2(2-((((S)-5-oxopyrrolidin-2-yl)methyl(amino) metyl)thiazol-5-yl)-[1,1′-bephenyl]-3-yl)3-((((S)-5-oxopyrrolidin-2-yl)methyl)amino)methyl)-4H-pyridol[1,2-a]pyrimidin-a-one

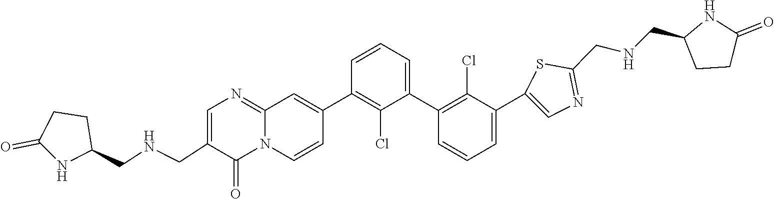 [SMILES: O=C1CC[C@@H](CNCc2ncc(-c3cccc(-c4cccc(-c5ccn6c(=O)c(CNC[C@@H]7CCC(=O)N7)cnc6c5)c4Cl)c3Cl)s2)N1]

### 5-((2,2′-Dichloro-3′-(3-formyl-4-oxo-4H-pyrido[1,2-a]pyrimidin-8-yl)-[1,1′-biphenyl]-3-yl)thiazole-2-carbaldehyde

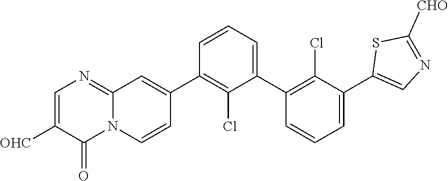 [SMILES: O=Cc1ncc(-c2cccc(-c3cccc(-c4ccn5c(=O)c(C=O)cnc5c4)c3Cl)c2Cl)s1]

A microwave vial was charged with 5-bromothiazole-2-carbaldehyde(0.04 g, 0.20 mmol), 8-(2,2′-dichloro-3′-(4,4,5,5,-tetramethyl-1,3,2-dioxaborolan-2-yl)-[1,1′-biphenyl]-3-yl)4-oxo-4H-pyido[1,2-a]pyrimdine-3-carbaldehyde (0.7 g 0.3 mmol), K₂Co₃ (0.06 g, 0.04 mmol), and Pd(dppf)₂Cl₂-DCM (0.02 g, 0.03 mmol). The flask was purged with nitrogen for 5 min, then 2 ml of dry dioxane was added, and the reaction was sparged with nitrogen for 5 5 min, then 2 ml of dry dioxane was added, and the reaction was sparged with nitrogen for 5 min. The mixture was stirred at 90° C. overnight thermally. The mixture was filtered through CELITE® and the filter cake was washed with EtOAc (3×40 mL). The filtrate was concentrated under reduced pressure. The residue was purified by normal phase flash SiO₂ chromatography using a gradient (0-5% methanol DCM) to afford 5-(2,2′-dichloro-3′-(3-formyl-4-oxo-4H-pyrido[1,2-a]pyrimidin-8-yl)-[1,1′-biphenyl]-3-yl)thiazole-2-carbaldehyde (0.028 g, 41% yield). MS: m/z found 506 [M+H]⁺.

### 8-(2,2′-Dichloro-3′-(2-(((((S)-5-oxopyrrolidin-2-yl)methyl)amino)methyl)thiazol-5-yl)-[1,1′-biphenyl]-3-yl)-3-(((((S)-5-oxopyrrolidin-2-yl)methyl)amino)methyl)-4H-pyrido[1,2-a]pyrimidin-4-one

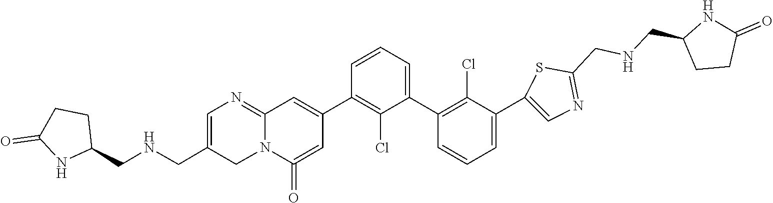

To a mixture of 5-(2,2′-dichloro-3′-(3-formyl-4-oxo-4H-pyrido[1,2-a]pyrimidin-8-yl)-[1,1′-biphenyl]-3-yl)thiazole-2-carbaldehyde (0.03 g, 0.06 mmol) and (S)-5-(aminomethyl)pyrrolidin-2-one (0.02 g, 0.18 mmol) in 1:1 THF/MeOH (6 mL) was added acetic acid (0.01 g, 0.18 mmol) and 4 Å molecular sieve (1 g). The mixture was stirred at r.t. for 3 hrs. Sodium cyanoborohydride (0.01 g, 0.21 mmol) was added and the mixture was stirred at r.t. for 1 hr. The reaction was concentrated, and residue purified by semi-prep HPLC (with 0.05% Formic acid modifier). ACN was evaporated off, and aqueous portion was lyophilized to give 5.5 mg (13%) of the product as bis formic acid salt affording 8-(2,2′-dichloro-3′-(2-(((((S)-5-oxopyrrolidin-2-yl)methyl)amino)methyl)thiazol-5-yl)-[1,1′-biphenyl]-3-yl)-3-(((((S)-5-oxopyrrolidin-2-yl)methyl)amino)methyl)-4H-pyrido[1,2-a]pyrimidin-4-one as a white solid. MS: m/z found 702 [M+H]⁺; ¹H NMR (400 MHz, Methanol-d₄) δ 9.11 (d, J=7.5 Hz, 1H), 8.50 (s, 4H), 8.39 (s, 1H), 7.89 (s, 1H), 7.74 (d, J=1.8 Hz, 1H), 7.65-7.58 (m, 2H), 7.55-7.48 (m, 2H), 7.38 (dd, J=7.5, 1.7 Hz, 1H), 4.19-4.09 (m, 2H), 3.89-3.77 (m, 4H), 2.83-2.67 (m, 4H), 2.37-2.22 (m, 5H), 1.26 (s, 3H).

### Example 441: 8-(2,2′-Dichloro-3′-(2-(((((S)-5-oxopyrrolidin-2-yl)methyl)amino) methyl)thiazol-4-yl)-[1,1′-biphenyl]-3-yl)-3-(((((S)-5-oxopyrrolidin-2-yl)methyl) amino)methyl)-4H-pyrido[1,2-a]pyrimidin-4-one

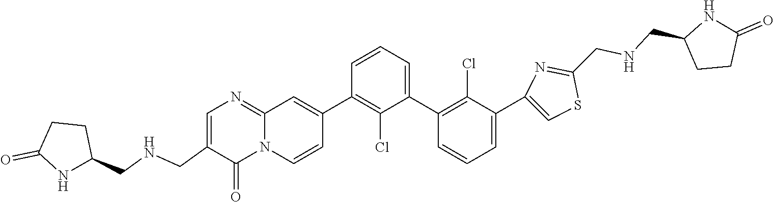 [SMILES: O=C1CC[C@@H](CNCc2nc(-c3cccc(-c4cccc(-c5ccn6c(=O)c(CNC[C@@H]7CCC(=O)N7)cnc6c5)c4Cl)c3Cl)cs2)N1]

This compound was prepared in a similar fashion to Example 442, using 4-bromothiazole-2-carbaldehyde and 8-(2,2′-dichloro-3′-(4,4,5,5-tetramethyl-1,3,2-dioxaborolan-2-yl)-[1,1′-biphenyl]-3-yl)-4-oxo-4H-pyrido[1,2-a]pyrimidine-3-carbaldehyde. MS: m/z found 702 [M+H]. ¹H NMR (400 MHz, Methanol-d₄) δ 9.12 (d, J=7.5, 0.8 Hz, 1H), 8.47-8.38 (m, 3H), 7.77 (t, 3H), 7.64-7.49 (m, 3H), 7.52-7.43 (m, 2H), 7.36 (dd, J=7.5, 1.7 Hz, 1H), 4.22-4.08 (m, 2H), 4.00-3.76 (m, 4H), 2.88-2.68 (m, 5H), 2.41-2.16 (m, 5H), 1.91-1.76 (m, 2H).

### Example 501: 8-(2,2′-Dichloro-3′-(3-(((((S)-5-oxopyrrolidin-2-yl)methyl)amino) methyl) pyrazolo[1,5-a]pyrimidin-6-yl)-[1,1′-biphenyl]-3-yl)-3-(((((S)-5-oxopyrrolidin-2-yl)methyl) amino)methyl)-4H-pyrido[1,2-a]pyrimidin-4-one

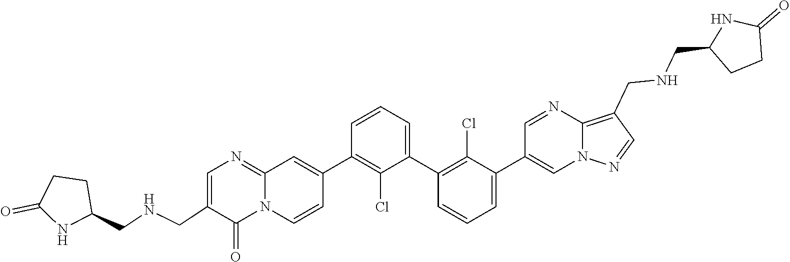 [SMILES: O=C1CC[C@@H](CNCc2cnc3cc(-c4cccc(-c5cccc(-c6cnc7c(CNC[C@@H]8CCC(=O)N8)cnn7c6)c5Cl)c4Cl)ccn3c2=O)N1]

This compound was prepared in a similar fashion to Example 442, using 6-bromopyrazolo[1,5-a]pyrimidine-3-carbaldehyde and 8-(2,2′-dichloro-3′-(4,4,5,5-tetramethyl-1,3,2-dioxaborolan-2-yl)-[1,1′-biphenyl]-3-yl)-4-oxo-4H-pyrido[1,2-a]pyrimidine-3-carbaldehyde. MS: m/z found 736 [M+H]. ¹H NMR (400 MHz, Methanol-d₄) δ 9.17-9.08 (m, 2H), 8.74 (d, J=2.1 Hz, 1H), 8.43 (s, 1H), 8.36 (s, 3H), 8.30 (s, 1H), 7.78 (d, J=1.8 Hz, 1H), 7.67-7.45 (m, 6H), 4.39-4.34 (m, 2H), 4.06-4.01 (m, 2H), 3.94-3.89 (m, 2H), 3.07-3.00 (m, 2H), 2.94-2.89 (m, 2H), 2.40-2.26 (m, 6H), 1.82 (s, 2H).

### Example 347: 8-(2,2′-Dichloro-3′-(6-methoxy-5-(((((S)-5-oxopyrrolidin-2-yl)methyl)amino)methyl)pyridin-2-yl)-[1,1′-biphenyl]-3-yl)-3-(((3R,4R)-3,4-dihydroxypyrrolidin-1-yl)methyl)-4H-pyrido[1,2-a]pyrimidin-4-one

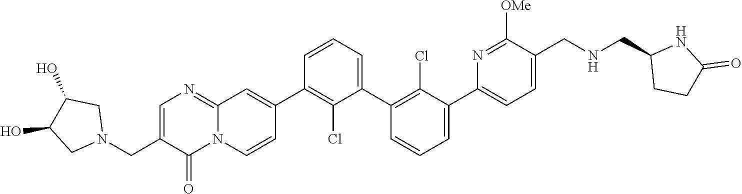 [SMILES: COc1nc(-c2cccc(-c3cccc(-c4ccn5c(=O)c(CN6C[C@@H](O)[C@H](O)C6)cnc5c4)c3Cl)c2Cl)ccc1CNC[C@@H]1CCC(=O)N1]

This compound was prepared in a similar fashion to Example 94, using (3R,4R)-pyrrolidine-3,4-diol and tert-butyl (S)-((6-(2,2′-dichloro-3′-(3-formyl-4-oxo-4H-pyrido[1,2-a]pyrimidin-8-yl)-[1,1′-biphenyl]-3-yl)-2-methoxypyridin-3-yl)methyl)((5-oxopyrrolidin-2-yl)methyl)carbamate. MS: m/z found 715 [M+H]⁺; ¹H NMR (400 MHz, Methanol-d₄) δ 9.00 (t, J=8.2, 4.4 Hz, 1H), 8.43 (s, 2H), 8.34 (d, J=2.6 Hz, 1H), 8.01-7.92 (m, 2H), 7.61-7.33 (m, 5H), 7.33-7.22 (m, 2H), 7.06 (dd, J=7.4, 3.0 Hz, 1H), 4.66 (s, 1H), 4.28-4.15 (m, 4H), 3.88 (d, J=1.5 Hz, 3H), 3.84-3.69 (m, 2H), 3.59 (d, J=12.1 Hz, 2H), 3.29 (d, J=14.8 Hz, 2H), 2.94-2.80 (m, 3H), 2.64-2.48 (m, 1H), 2.43-2.28 (m, 1H), 2.26-2.15 (m, 1H).

### Example 346: (S)-8-(2,2′-Dichloro-3′-(6-methoxy-5-((((5-oxopyrrolidin-2-yl)methyl) amino)methyl)pyridin-2-yl)-[1,1′-biphenyl]-3-yl)-3-((6-hydroxy-2-azaspiro[3.3]heptan-2-yl)methyl)-4H-pyrido[1,2-a]pyrimidin-4-one

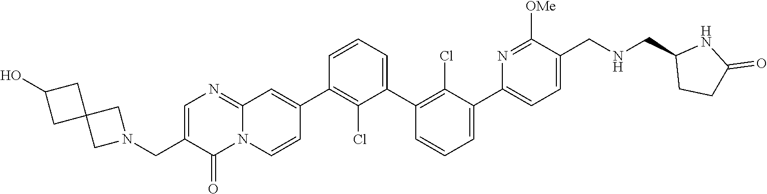 [SMILES: COc1nc(-c2cccc(-c3cccc(-c4ccn5c(=O)c(CN6CC7(CC(O)C7)C6)cnc5c4)c3Cl)c2Cl)ccc1CNC[C@@H]1CCC(=O)N1]

This compound was prepared in a similar fashion to Example 94, using 2-azaspiro[3.3]heptan-6-ol and tert-butyl (S)-((6-(2,2′-dichloro-3′-(3-formyl-4-oxo-4H-pyrido[1,2-a]pyrimidin-8-yl)-[1,1′-biphenyl]-3-yl)-2-methoxypyridin-3-yl)methyl)((5-oxopyrrolidin-2-yl)methyl)carbamate. MS: m/z found 725 [M+H]⁺; ¹H NMR (400 MHz, Methanol-d₄) δ 8.97 (dd, J=8.1, 3.5 Hz, 1H), 8.44 (s, 2H), 8.27 (d, J=2.5 Hz, 1H), 7.95 (s, 2H), 7.57 (dd, J=7.4, 2.9 Hz, 1H), 7.54-7.33 (m, 4H), 7.32-7.22 (m, 2H), 7.06 (dd, J=7.4, 4.1 Hz, 1H), 4.60 (s, 2H), 4.15-4.05 (m, 2H), 4.02 (d, J=12.9 Hz, 3H), 3.88 (s, 3H), 3.76 (d, 2H), 2.92-2.78 (m, 3H), 2.64-2.48 (m, 3H), 2.40-2.29 (m, 1H), 2.25-2.17 (m, 1H), 2.13-2.03 (m, 1H).

### Example 345: (S)-8-(2,2′-Dichloro-3′-(6-methoxy-5-((((5-oxopyrrolidin-2-yl)methyl)amino)methyl)pyridin-2-yl)-[1,1′-biphenyl]-3-yl)-3-(((2-methoxy-2-methylpropyl)amino)methyl)-4H-pyrido[1,2-a]pyrimidin-4-one

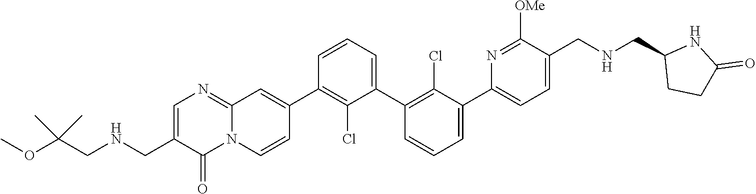 [SMILES: COc1nc(-c2cccc(-c3cccc(-c4ccn5c(=O)c(CNCC(C)(C)OC)cnc5c4)c3Cl)c2Cl)ccc1CNC[C@@H]1CCC(=O)N1]

This compound was prepared in a similar fashion to Example 94, using 2-methoxy-2-methylpropan-1-amine and tert-butyl (S)-((6-(2,2′-dichloro-3′-(3-formyl-4-oxo-4H-pyrido[1,2-a]pyrimidin-8-yl)-[1,1′-biphenyl]-3-yl)-2-methoxypyridin-3-yl)methyl)((5-oxopyrrolidin-2-yl)methyl)carbamate. MS: m/z found 715 [M+H]⁺; ¹H NMR (400 MHz, Methanol-d₄) δ 9.14 (d, J=7.4, 0.7 Hz, 1H), 8.47 (s, 1H), 8.39 (s, 2H), 7.85-7.74 (m, 2H), 7.71-7.53 (m, 4H), 7.54-7.44 (m, 2H), 7.37 (d, J=7.6, 1.7 Hz, 1H), 7.25 (d, J=7.5 Hz, 1H), 4.26 (s, 2H), 4.09-3.96 (m, 5H), 3.93-3.88 (m, 1H), 3.22 (s, 2H), 3.09 (s, 2H), 2.99-2.84 (m, 2H), 2.33-2.25 (m, 2H), 1.89-1.75 (m, 1H), 1.24 (s, 6H).

### Example 301: (S)-8-(2,2′-Dichloro-3′-(6-methoxy-5-((((5-oxopyrrolidin-2-yl)methyl) amino)methyl)pyridin-2-yl)-[1,1′-biphenyl]-3-yl)-3-((((1-(hydroxymethyl) cyclopropyl)methyl)amino)methyl)-4H-pyrido[1,2-a]pyrimidin-4-one

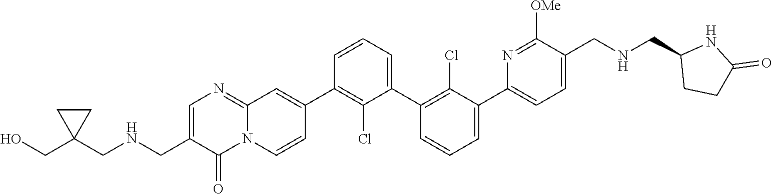 [SMILES: COc1nc(-c2cccc(-c3cccc(-c4ccn5c(=O)c(CNCC6(CO)CC6)cnc5c4)c3Cl)c2Cl)ccc1CNC[C@@H]1CCC(=O)N1]

This compound was prepared in a similar fashion to Example 94, using (1-(aminomethyl) cyclopropyl)methanol and tert-butyl (S)-((6-(2,2′-dichloro-3′-(3-formyl-4-oxo-4H-pyrido[1,2-a]pyrimidin-8-yl)-[1,1′-biphenyl]-3-yl)-2-methoxypyridin-3-yl)methyl)((5-oxopyrrolidin-2-yl)methyl)carbamate. MS: m/z found 713 [M+H]⁺; ¹H NMR (400 MHz, Methanol-d₄) δ 9.13 (d, 1H), 8.47-8.38 (m, 3H), 7.84-7.72 (m, 2H), 7.65-7.57 (m, 2H), 7.60-7.55 (m, 1H), 7.50-7.45 (m, 2H), 7.37 (dd, J=7.6, 1.7 Hz, 1H), 7.24 (d, J=7.4 Hz, 1H), 4.24 (s, 2H), 3.98 (d, J=12.1 Hz, 5H), 3.88 (s, 1H), 3.51 (s, 2H), 3.16 (s, 2H), 2.93-2.78 (m, 2H), 2.39-2.21 (m, 3H), 1.87-1.74 (m, 1H), 0.71-0.58 (m, 4H).

### Example 300: (S)-8-(2,2′-Dichloro-3′-(6-methoxy-5-((((5-oxopyrrolidin-2-yl)methyl)amino)methyl)pyridin-2-yl)-[1,1′-biphenyl]-3-yl)-3-(((2,2-difluoro-3-hydroxypropyl)amino)methyl)-4H-pyrido[1,2-a]pyrimidin-4-one

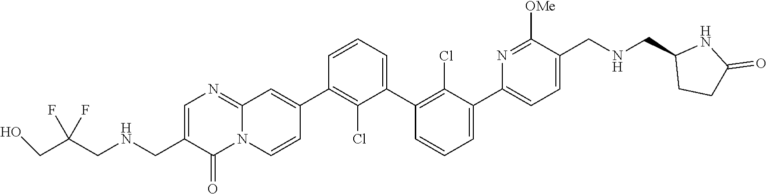 [SMILES: COc1nc(-c2cccc(-c3cccc(-c4ccn5c(=O)c(CNCC(F)(F)CO)cnc5c4)c3Cl)c2Cl)ccc1CNC[C@@H]1CCC(=O)N1]

This compound was prepared in a similar fashion to Example 94, using 3-amino-2,2-difluoropropan-1-ol and tert-butyl (S)-((6-(2,2′-dichloro-3′-(3-formyl-4-oxo-4H-pyrido[1,2-a]pyrimidin-8-yl)-[1,1′-biphenyl]-3-yl)-2-methoxypyridin-3-yl)methyl)((5-oxopyrrolidin-2-yl)methyl)carbamate. MS: m/z found 723 [M+H]⁺; H NMR (400 MHz, Methanol-d₄) δ 9.09 (d, J=7.4, 0.7 Hz, 1H), 8.43-8.35 (m, 2H), 7.78-7.70 (m, 2H), 7.66-7.51 (m, 3H), 7.53-7.43 (m, 3H), 7.37 (d, J=7.5, 1.8, 0.6 Hz, 1H), 7.25 (d, J=7.5 Hz, 1H), 4.03-3.91 (m, 5H), 3.88 (s, 3H), 3.73 (t, J=13.1 Hz, 2H), 3.06 (t, J=14.6 Hz, 2H), 2.89-2.75 (m, 2H), 2.39-2.21 (m, 3H), 1.87-1.74 (m, 1H).

### Example 274: 3-(((2-Cyclopropyl-2-hydroxyethyl)amino)methyl)-8-(2,2′-dichloro-3′-(6-methoxy-5-(((((S)-5-oxopyrrolidin-2-yl)methyl)amino)methyl)pyridin-2-yl)-[1,1′-biphenyl]-3-yl)-4H-pyrido[1,2-a]pyrimidin-4-one

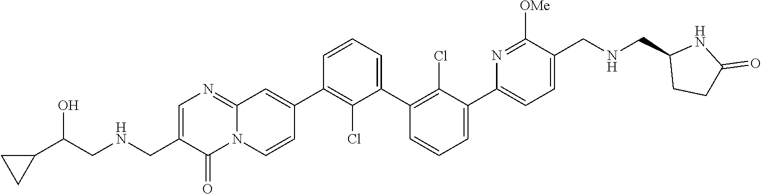 [SMILES: COc1nc(-c2cccc(-c3cccc(-c4ccn5c(=O)c(CNCC(O)C6CC6)cnc5c4)c3Cl)c2Cl)ccc1CNC[C@@H]1CCC(=O)N1]

This compound was prepared in a similar fashion to Example 94, using 2-amino-1-cyclopropylethan-1-ol and tert-butyl (S)-((6-(2,2′-dichloro-3′-(3-formyl-4-oxo-4H-pyrido[1,2-a]pyrimidin-8-yl)-[1,1′-biphenyl]-3-yl)-2-methoxypyridin-3-yl)methyl)((5-oxopyrrolidin-2-yl)methyl)carbamate. MS: m/z found 713 [M+H]⁺; ¹H NMR (400 MHz, Methanol-d₄) δ 9.13 (d, J=7.4, 3.0 Hz, 1H), 8.46 (s, 2H), 7.84-7.75 (m, 1H), 7.73 (d, J=7.5 Hz, 1H), 7.69-7.57 (m, 3H), 7.61-7.42 (m, 3H), 7.35 (d, J=7.6, 1.7 Hz, 1H), 7.22 (d, J=7.4 Hz, 1H), 4.24 (s, 2H), 3.98 (s, 2H), 3.98-3.78 (m, 4H), 3.09-2.98 (m, 1H), 2.87-2.72 (m, 3H), 2.37-2.19 (m, 3H), 1.85-1.72 (m, 1H), 1.23 (s, 1H), 0.83 (d, J=8.6 Hz, 1H), 0.58-0.43 (m, 2H), 0.42-0.33 (m, 1H), 0.29-0.20 (m, 1H).

### Example 234: (S)-8-(2,2′-Dichloro-3′-(6-methoxy-5-((((5-oxopyrrolidin-2-yl)methyl) amino)methyl)pyridin-2-yl)-[1,1′-biphenyl]-3-yl)-3-((((1-hydroxycyclopropyl) methyl)amino)methyl)-4H-pyrido[1,2-a]pyrimidin-4-one

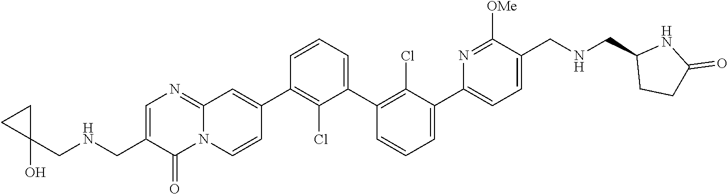 [SMILES: COc1nc(-c2cccc(-c3cccc(-c4ccn5c(=O)c(CNCC6(O)CC6)cnc5c4)c3Cl)c2Cl)ccc1CNC[C@@H]1CCC(=O)N1]

This compound was prepared in a similar fashion to Example 94, using 1-(aminomethyl)cyclopropan-1-ol and tert-butyl (S)-((6-(2,2′-dichloro-3′-(3-formyl-4-oxo-4H-pyrido[1,2-a]pyrimidin-8-yl)-[1,1′-biphenyl]-3-yl)-2-methoxypyridin-3-yl)methyl)((5-oxopyrrolidin-2-yl)methyl)carbamate. MS: m/z found 699 [M+H]⁺; H NMR (400 MHz, Methanol-d₄) δ 9.14 (d, J=7.4 Hz, 1H), 8.48 (s, 1H), 8.40 (s, 2H), 7.84-7.71 (m, 2H), 7.66-7.52 (m, 4H), 7.53-7.42 (m, 2H), 7.37 (d, J=7.5, 1.7 Hz, 1H), 7.24 (d, J=7.5, 1.5 Hz, 1H), 4.31 (s, 2H), 4.00 (d, J=1.0 Hz, 5H), 3.88 (s, 1H), 3.16 (s, 2H), 2.86 (d, J=7.8 Hz, 2H), 2.36-2.18 (m, 3H), 1.86-1.74 (m, 1H), 0.91-0.81 (m, 2H), 0.73-0.65 (m, 2H).

### Example 233: (S)-8-(2,2′-Dichloro-3′-(6-methoxy-5-((((5-oxopyrrolidin-2-yl)methyl) amino)methyl)pyridin-2-yl)-[1,1′-biphenyl]-3-yl)-3-((((1-hydroxycyclobutyl) methyl)amino)methyl)-4H-pyrido[1,2-a]pyrimidin-4-one

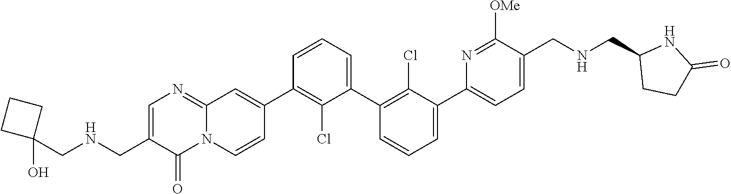 [SMILES: COc1nc(-c2cccc(-c3cccc(-c4ccn5c(=O)c(CNCC6(O)CCC6)cnc5c4)c3Cl)c2Cl)ccc1CNC[C@@H]1CCC(=O)N1]

This compound was prepared in a similar fashion to Example 94, using 1-(aminomethyl)cyclopropan-1-ol and tert-butyl (S)-((6-(2,2′-dichloro-3′-(3-formyl-4-oxo-4H-pyrido[1,2-a]pyrimidin-8-yl)-[1,1′-biphenyl]-3-yl)-2-methoxypyridin-3-yl)methyl)((5-oxopyrrolidin-2-yl)methyl)carbamate. MS: m/z found 713 [M+H]⁺; 1 H NMR (400 MHz, Methanol-d₄) δ 9.08 (d, J=7.4 Hz, 1H), 8.37 (s, 1H), 7.75-7.66 (m, 2H), 7.65-7.51 (m, 3H), 7.53-7.42 (m, 3H), 7.35 (d, J=7.6, 1.7 Hz, 1H), 7.20 (d, J=7.4 Hz, 1H), 3.97 (s, 3H), 3.86 (s, 2H), 3.85-3.72 (m, 3H), 2.73 (s, 2H), 2.69-2.57 (m, 2H), 2.34-2.18 (m, 3H), 2.12-1.93 (m, 4H), 1.81-1.67 (m, 2H), 1.52 (t, J=19.4, 9.5 Hz, 1H).

### Example 208: 8-(2′-Chloro-3′-(6-methoxy-5-(((((S)-5-oxopyrrolidin-2-yl)methyl)amino) methyl)pyridin-2-yl)-2-(trifluoromethyl)-[1,1′-biphenyl]-3-yl)-3-(((((S)-5-oxopyrrolidin-2-yl)methyl)amino)methyl)-4H-pyrido[1,2-a]pyrimidin-4-one

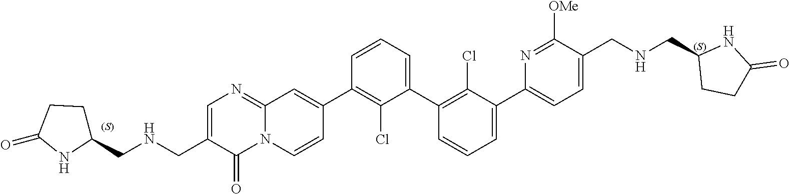

This compound was prepared in a similar fashion to Example 140, but using 1,3-dibromo-2-(trifluoromethyl)benzene and (3-bromo-4-oxo-4H-pyrido[1,2-a]pyrimidin-8-yl)boronic acid to couple first then combining with 6-(2-chloro-3-(4,4,5,5-tetramethyl-1,3,2-dioxaborolan-2-yl)phenyl)-2-methoxynicotinaldehyde. MS: m/z found 760 [M+H. ¹H NMR (400 MHz, Methanol-d₄) δ 9.16 (d, J=7.4, 0.8 Hz, 1H), 8.46 (s, 1H), 8.33 (s, 1H), 7.85-7.77 (m, 2H), 7.73-7.65 (m, 2H), 7.60-7.49 (m, 3H), 7.43 (d, J=7.6, 1.8 Hz, 1H), 7.30 (d, J=7.5, 4.0 Hz, 1H), 4.15-3.97 (m, 6H), 3.96-3.91 (m, 2H), 3.04-2.86 (m, 4H), 2.43-2.25 (m, 7H), 1.92-1.79 (m, 2H).

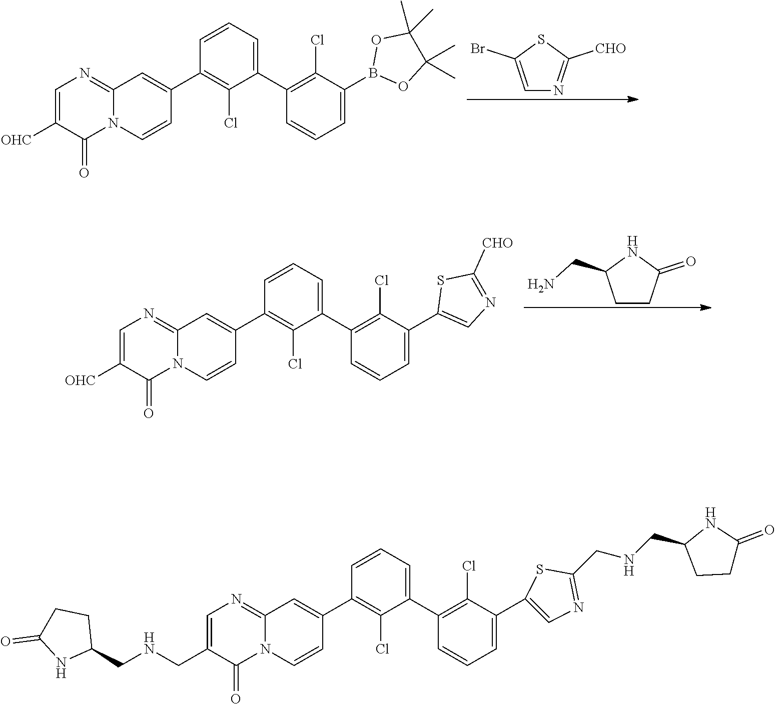

### Example 442: 8-(2,2′-dichloro-3′-(2-(((((S)-5-oxopyrrolidin-2-yl)methyl)amino) methyl)thiazol-5-yl)-[1,1′-biphenyl]-3-yl)-3-(((((S)-5-oxopyrrolidin-2-yl)methyl)amino)methyl)-4H-pyrido[1,2-a]pyrimidin-4-one

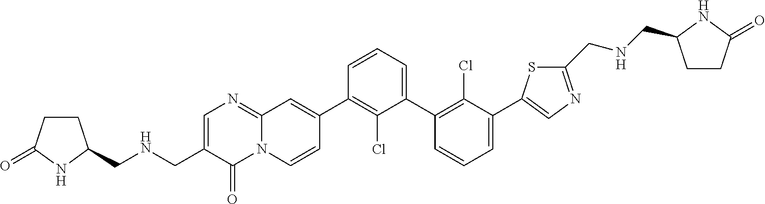 [SMILES: O=C1CC[C@@H](CNCc2ncc(-c3cccc(-c4cccc(-c5ccn6c(=O)c(CNC[C@@H]7CCC(=O)N7)cnc6c5)c4Cl)c3Cl)s2)N1]

### 5-(2,2′-Dichloro-3′-(3-formyl-4-oxo-4H-pyrido[1,2-a]pyrimidin-8-yl)-[1,1′-biphenyl]-3-yl)thiazole-2-carbaldehyde

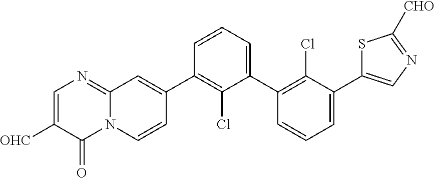 [SMILES: O=Cc1ncc(-c2cccc(-c3cccc(-c4ccn5c(=O)c(C=O)cnc5c4)c3Cl)c2Cl)s1]

A microwave vial was charged with 5-bromothiazole-2-carbaldehyde (0.04 g, 0.20 mmol), 8-(2,2′-dichloro-3′-(4,4,5,5-tetramethyl-1,3,2-dioxaborolan-2-yl)-[1,1′-biphenyl]-3-yl)-4-oxo-4H-pyrido[1,2-a]pyrimidine-3-carbaldehyde (0.07 g, 0.13 mmol), K₂CO₃ (0.06 g, 0.40 mmol), and Pd(dppf)₂Cl₂-DCM (0.02 g, 0.03 mmol). The flask was purged with nitrogen for 5 min, then 2 ml of dry dioxane was added, and the reaction was sparged with nitrogen for 5 min. The mixture was stirred at 90° C. overnight thermally. The mixture was filtered through CELITE® and the filter cake was washed with EtOAc (3×40 mL). The filtrate was concentrated under reduced pressure. The residue was purified by normal phase flash SiO₂ chromatography using a gradient (0-5% methanol DCM) to afford 5-(2,2′-dichloro-3′-(3-formyl-4-oxo-4H-pyrido[1,2-a]pyrimidin-8-yl)-[1,1′-biphenyl]-3-yl)thiazole-2-carbaldehyde (0.028 g, 41% yield). MS: m/z found 506 [M+H]⁺.

### 8-(2,2′-Dichloro-3′-(2-(((((S)-5-oxopyrrolidin-2-yl)methyl)amino)methyl)thiazol-5-yl)-[1,1′-biphenyl]-3-yl)-3-(((((S)-5-oxopyrrolidin-2-yl)methyl)amino)methyl)-4H-pyrido[1,2-a]pyrimidin-4-one

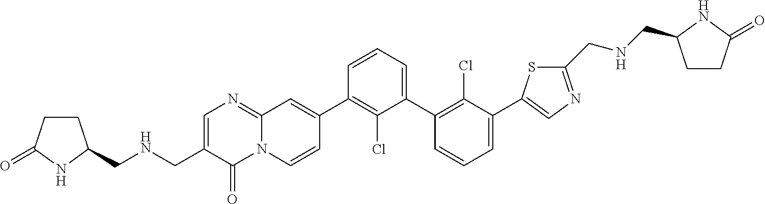 [SMILES: O=C1CC[C@@H](CNCc2ncc(-c3cccc(-c4cccc(-c5ccn6c(=O)c(CNC[C@@H]7CCC(=O)N7)cnc6c5)c4Cl)c3Cl)s2)N1]

To a mixture of 5-(2,2′-dichloro-3′-(3-formyl-4-oxo-4H-pyrido[1,2-a]pyrimidin-8-yl)-[1,1′-biphenyl]-3-yl)thiazole-2-carbaldehyde (0.03 g, 0.06 mmol) and (S)-5-(aminomethyl)pyrrolidin-2-one (0.02 g, 0.18 mmol) in 1:1 THF/MeOH (6 mL) was added acetic acid (0.01 g, 0.18 mmol) and 4 Å molecular sieve (1 g). The mixture was stirred at r.t. for 3 hrs. Sodium cyanoborohydride (0.01 g, 0.21 mmol) was added and the mixture was stirred at r.t. for 1 hr. The reaction was concentrated, and residue purified by semi-prep HPLC (with 0.05% Formic acid modifier). The acetonitrile was evaporated off, and the aqueous portion was lyophilized to give 5.5 mg (13%) of the product as bis formic acid salt affording 8-(2,2′-dichloro-3′-(2-(((((S)-5-oxopyrrolidin-2-yl)methyl)amino)methyl)thiazol-5-yl)-[1,1′-biphenyl]-3-yl)-3-(((((S)-5-oxopyrrolidin-2-yl)methyl)amino)methyl)-4H-pyrido[1,2-a]pyrimidin-4-one as a white solid. MS: m/z found 702 [M+H]⁺; ¹H NMR (400 MHz, Methanol-d₄) δ 9.11 (d, J=7.5 Hz, 1H), 8.50 (s, 4H), 8.39 (s, 1H), 7.89 (s, 1H), 7.74 (d, J=1.8 Hz, 1H), 7.65-7.58 (m, 2H), 7.55-7.48 (m, 2H), 7.38 (dd, J=7.5, 1.7 Hz, 1H), 4.19-4.09 (m, 2H), 3.89-3.77 (m, 4H), 2.83-2.67 (m, 4H), 2.37-2.22 (m, 5H), 1.26 (s, 3H).

### Example 441: 8-(2,2′-Dichloro-3′-(2-(((((S)-5-oxopyrrolidin-2-yl)methyl)amino) methyl)thiazol-4-yl)-[1,1′-biphenyl]-3-yl)-3-(((((S)-5-oxopyrrolidin-2-yl)methyl) amino)methyl)-4H-pyrido[1,2-a]pyrimidin-4-one

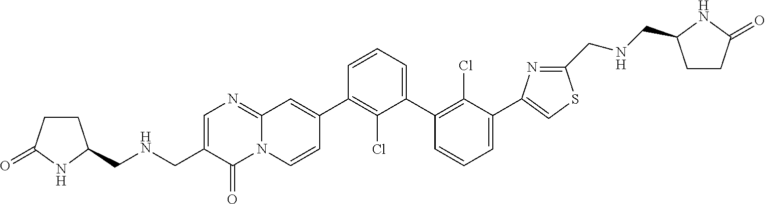 [SMILES: O=C1CC[C@@H](CNCc2nc(-c3cccc(-c4cccc(-c5ccn6c(=O)c(CNC[C@@H]7CCC(=O)N7)cnc6c5)c4Cl)c3Cl)cs2)N1]

This compound was prepared in a similar fashion to Example 442, using 4-bromothiazole-2-carbaldehyde and 8-(2,2′-dichloro-3′-(4,4,5,5-tetramethyl-1,3,2-dioxaborolan-2-yl)-[1,1′-biphenyl]-3-yl)-4-oxo-4H-pyrido[1,2-a]pyrimidine-3-carbaldehyde. MS: m/z found 702 [M+H]. ¹H NMR (400 MHz, Methanol-d₄) δ 9.12 (d, J=7.5, 0.8 Hz, 1H), 8.47-8.38 (m, 3H), 7.77 (t, 3H), 7.64-7.49 (m, 3H), 7.52-7.43 (m, 2H), 7.36 (dd, J=7.5, 1.7 Hz, 1H), 4.22-4.08 (m, 2H), 4.00-3.76 (m, 4H), 2.88-2.68 (m, 5H), 2.41-2.16 (m, 5H), 1.91-1.76 (m, 2H).

### Example 501: 8-(2,2′-Dichloro-3′-(3-(((((S)-5-oxopyrrolidin-2-yl)methyl)amino) methyl) pyrazolo[1,5-a]pyrimidin-6-yl)-[1,1′-biphenyl]-3-yl)-3-(((((S)-5-oxopyrrolidin-2-yl)methyl) amino)methyl)-4H-pyrido[1,2-a]pyrimidin-4-one

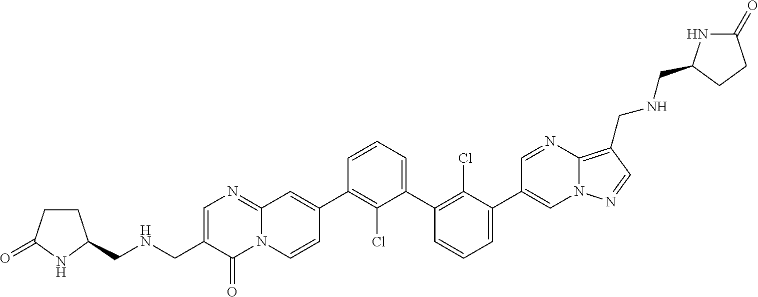 [SMILES: O=C1CC[C@@H](CNCc2cnc3cc(-c4cccc(-c5cccc(-c6cnc7c(CNC[C@@H]8CCC(=O)N8)cnn7c6)c5Cl)c4Cl)ccn3c2=O)N1]

This compound was prepared in a similar fashion to Example 442, using 6-bromopyrazolo[1,5-a]pyrimidine-3-carbaldehyde and 8-(2,2′-dichloro-3′-(4,4,5,5-tetramethyl-1,3,2-dioxaborolan-2-yl)-[1,1′-biphenyl]-3-yl)-4-oxo-4H-pyrido[1,2-a]pyrimidine-3-carbaldehyde. MS: m/z found 736 [M+H]. ¹H NMR (400 MHz, Methanol-d₄) δ 9.17-9.08 (m, 2H), 8.74 (d, J=2.1 Hz, 1H), 8.43 (s, 1H), 8.36 (s, 3H), 8.30 (s, 1H), 7.78 (d, J=1.8 Hz, 1H), 7.67-7.45 (m, 6H), 4.39-4.34 (m, 2H), 4.06-4.01 (m, 2H), 3.94-3.89 (m, 2H), 3.07-3.00 (m, 2H), 2.94-2.89 (m, 2H), 2.40-2.26 (m, 6H), 1.82 (s, 2H).

### Example 223: (S)-8-(2,2′-dichloro-3′-(6-methoxy-5-((((5-oxopyrrolidin-2-yl)-methyl)-amino)methyl)-pyridin-2-yl)-[1,1′-biphenyl]-3-yl)-3-(((4,4,5,5,5-pentafluoropentyl)amino)-methyl)-4H-pyrido[1,2-a]pyrimidin-4-one

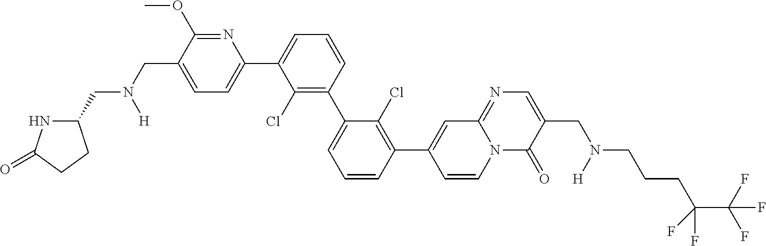 [SMILES: COc1nc(-c2cccc(-c3cccc(-c4ccn5c(=O)c(CNCCCC(F)(F)C(F)(F)F)cnc5c4)c3Cl)c2Cl)ccc1CNC[C@@H]1CCC(=O)N1]

### tert-Butyl (S)-((6-(2,2′-dichloro-3′-(4-oxo-3-(((4,4,5,5,5-pentafluoropentyl)amino)methyl)-4H-pyrido[1,2-a]pyrimidin-8-yl)-[1,1′-biphenyl]-3-yl)-2-methoxypyridin-3-yl)methyl)((5-oxopyrrolidin-2-yl)methyl)carbamate

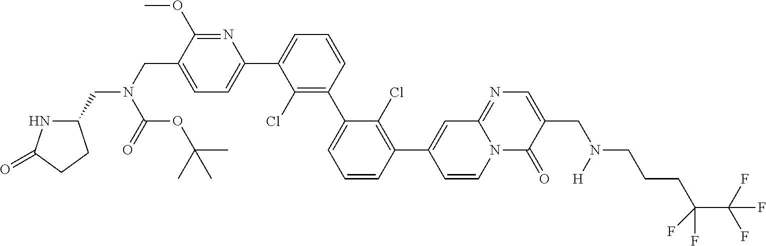 [SMILES: COc1nc(-c2cccc(-c3cccc(-c4ccn5c(=O)c(CNCCCC(F)(F)C(F)(F)F)cnc5c4)c3Cl)c2Cl)ccc1CN(C[C@@H]1CCC(=O)N1)C(=O)OC(C)(C)C]

To a solution of tert-butyl N-[[6-[2-chloro-3-[2-chloro-3-(3-formyl-4-oxo-pyrido-[1,2-a]-pyrimidin-8-yl)phenyl]-phenyl]-2-methoxy-3-pyridyl]methyl]-N-[[(2S)-5-oxopyrrolidin-2-yl]-methyl]carbamate (30 mg, 0.04 mmol) and 4,4,5,5,5-pentafluoropentan-1-amine hydrochloride (12.5 mg, 0.07 mmol) in dioxane/methanol (1:1, 2 mL) were added acetic acid (10 mg, 0.16 mmol) and DIEA (0.17 mL, 1 mmol). Reaction was stirred overnight at 60° C., then sodium triacetoxyborohydride (20 mg, 0.094 mmol) was added and the mixture was stirred for 2 h at room temperature. Reaction was diluted with water (10 mL) and extracted with dichloromethane (2×20 mL). The combined organic layer was washed with brine (30 mL), dried over sodium sulfate (10 g), filtered, and concentrated to afford desired product (35 mg, crude) as an orange solid. MS: m/z found 891 [M+H]⁺. Material was used for the next reaction without further purification.

### (S)-8-(2,2′-Dichloro-3′-(6-methoxy-5-((((5-oxopyrrolidin-2-yl)-methyl)-amino)methyl)-pyridin-2-yl)-[1,1′-biphenyl]-3-yl)-3-(((4,4,5,5,5-pentafluoropentyl)amino)-methyl)-4H-pyrido[1,2-a]pyrimidin-4-one

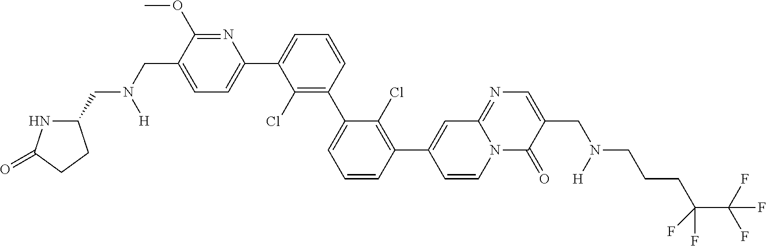 [SMILES: COc1nc(-c2cccc(-c3cccc(-c4ccn5c(=O)c(CNCCCC(F)(F)C(F)(F)F)cnc5c4)c3Cl)c2Cl)ccc1CNC[C@@H]1CCC(=O)N1]

tert-Butyl N-[[6-[2-chloro-3-[2-chloro-3-[4-oxo-3-[(4,4,5,5,5-pentafluoropentylamino)methyl]pyrido[1,2-a]pyrimidin-8-yl]phenyl]phenyl]-2-methoxy-3-pyridyl]methyl]-N-[[(2S)-5-oxopyrrolidin-2-yl]methyl]carbamate (30 mg, 0.03 mmol) was dissolved in dioxane (3 mL). HCl solution (0.3 mL, 0.3 mmol, 4 M HCl in dioxane) was added and the reaction was stirred for 3 hours at room temperature. Solvent was evaporated and crude product was purified by reverse phase HPLC to afford 8-[2-chloro-3-[2-chloro-3-[6-methoxy-5-[[[(2S)-5-oxopyrrolidin-2-yl]methylamino]methyl]-2-pyridyl]phenyl]phenyl]-3-[(4,4,5,5,5-pentafluoropentylamino)methyl]pyrido[1,2-a]pyrimidin-4-one (bis-formic acid salt) as an off white solid (8.1 mg, 30.4% yield). m/z: 790 [M+H]⁺ observed. ¹H NMR (400 MHz, CD₃OD) δ9.13 (dd, J=7.2 Hz, J=2.4 Hz, 1H), 8.43 (s, 1H), 7.79 (s, 1H), 7.73 (dd, J=7.2 Hz, J=2.4 Hz, 1H), 7.66-7.48 (m, 6H), 7.40-7.37 (m, 1H), 7.24 (dd, J=7.2 Hz, J=2.4 Hz, 1H), 4.02-3.99 (m, 5H), 3.88-3.68 (m, 3H), 2.91-2.85 (m, 2H), 2.78-2.65 (m, 2H), 2.36-2.17 (m, 5H), 1.96-1.76 (m, 3H).

### Example 224: (S)-8-(2,2′-dichloro-3′-(6-methoxy-5-((((5-oxopyrrolidin-2-yl)-methyl)amino)-methyl)-pyridin-2-yl)-[1,1′-biphenyl]-3-yl)-3-(((1,1-dioxidotetrahydro-2H-thiopyran-4-yl)-amino)methyl)-4H-pyrido[1,2-a]-pyrimidin-4-one

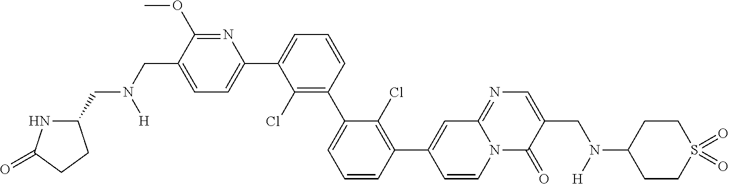 [SMILES: COc1nc(-c2cccc(-c3cccc(-c4ccn5c(=O)c(CNC6CCS(=O)(=O)CC6)cnc5c4)c3Cl)c2Cl)ccc1CNC[C@@H]1CCC(=O)N1]

Penultimate intermediate was prepared in the same manner as described for Example 223, utilizing intermediate (S)-tert-butyl ((6-(2,2′-dichloro-3′-(3-formyl-4-oxo-4H-pyrido[1,2-a]pyrimidin-8-yl)-[1,1′-biphenyl]-3-yl)-2-methoxypyridin-3-yl)methyl)((5-oxopyrrolidin-2-yl)methyl)carbamate. 4,4,5,5,5-pentafluoropentan-1-amine hydrochloride was replaced by 4-aminotetrahydro-2H-thiopyran 1,1-dioxide hydrochloride.

tert-Butyl N-[[6-[2-chloro-3-[2-chloro-3-[3-[[(1,1-dioxothian-4-yl)amino]methyl]-4-oxo-pyrido[1,2-a]pyrimidin-8-yl]phenyl]phenyl]-2-methoxy-3-pyridyl]methyl]-N-[[(2S)-5-oxopyrrolidin-2-yl]methyl]carbamate (30 mg, 0.035 mmol) was dissolved in dioxane (3 mL). HCl solution (0.3 mL, 0.3 mmol, 4 M HCl in dioxane) was added and the reaction was stirred for 3 hours at room temperature. Solvent was evaporated and crude product was purified by reverse phase HPLC to afford 8-[2-chloro-3-[2-chloro-3-[6-methoxy-5-[[[(2S)-5-oxopyrrolidin-2-yl]methylamino]methyl]-2-pyridyl]phenyl]phenyl]-3-[[(1,1-dioxothian-4-yl)amino]methyl]pyrido[1,2-a]pyrimidin-4-one (bis-formic acid salt) as a white solid (14 mg, 52.8% yield). m/z: 762 [M+H]⁺ observed. ¹H NMR (400 MHz, CD₃OD) δ9.12 (d, J=7.2 Hz, 1H), 8.45 (s, 1H), 7.80 (d, J=7.2 Hz, 1H), 7.77 (s, 1H), 7.66-7.48 (m, 6H), 7.39 (dd, J=7.2 Hz, J=1.6 Hz, 1H), 7.28 (d, J=7.2 Hz, 1H), 4.08-3.90 (m, 8H), 3.22-3.07 (m, 5H), 2.98-2.88 (m, 2H), 2.40-2.32 (m, 5H), 2.16-2.09 (m, 2H), 1.90-1.81 (m, 1H).

### Example 225: 8-(2,2′-dichloro-3′-(6-methoxy-5-(((((S)-5-oxopyrrolidin-2-yl)methyl)-amino)-methyl)-pyridin-2-yl)-[1,1′-biphenyl]-3-yl)-3-(((((1S,2R)-2-(hydroxymethyl)-cyclopropyl)methyl)amino)methyl)-4H-pyrido[1,2-a]pyrimidin-4-one

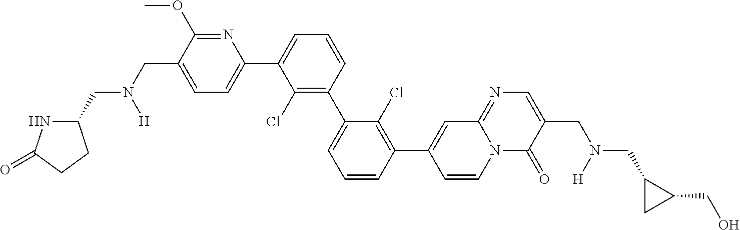 [SMILES: COc1nc(-c2cccc(-c3cccc(-c4ccn5c(=O)c(CNC[C@H]6C[C@H]6CO)cnc5c4)c3Cl)c2Cl)ccc1CNC[C@@H]1CCC(=O)N1]

Penultimate intermediate was prepared in the same manner as described for Example 223, utilizing intermediate (S)-tert-butyl ((6-(2,2′-dichloro-3′-(3-formyl-4-oxo-4H-pyrido[1,2-a]pyrimidin-8-yl)-[1,1′-biphenyl]-3-yl)-2-methoxypyridin-3-yl)methyl)((5-oxopyrrolidin-2-yl)methyl)carbamate. 4,4,5,5,5-pentafluoropentan-1-amine hydrochloride was replaced by rac-((1R,2S)-2-(aminomethyl)cyclopropyl)-methanol hydrochloride.

tert-Butyl N-[[6-[2-chloro-3-[2-chloro-3-[4-oxo-3-[[[rac-(1S,2R)-2-(hydroxymethyl)cyclopropyl]methylamino]methyl]pyrido[1,2-a]pyrimidin-8-yl]phenyl]phenyl]-2-methoxy-3-pyridyl]methyl]-N-[[(2S)-5-oxopyrrolidin-2-yl]methyl]carbamate (25 mg, 0.03 mmol) was dissolved in dioxane (3 mL). HCl solution (0.3 mL, 0.3 mmol, 4 M HCl in dioxane) was added and the reaction was stirred for 3 hours at room temperature. Solvent was evaporated and crude product was purified by reverse phase HPLC to afford 8-[2-chloro-3-[2-chloro-3-[6-methoxy-5-[[[(2S)-5-oxopyrrolidin-2-yl]methylamino]methyl]-2-pyridyl]phenyl]phenyl]-3-[[[rac-(1S,2R)-2-(hydroxymethyl)cyclopropyl]methylamino]methyl]pyrido[1,2-a]pyrimidin-4-one (bis-formic acid salt) as a white solid (4 mg, 18.2% yield). m/z: 714 [M+H]⁺ observed. ¹H NMR (400 MHz, CD₃OD) δ9.09 (d, J=1.2 Hz, 1H), 8.38 (brs, 1H), 8.13 (dd, J=9.2 Hz, J=1.2 Hz, 1H), 7.84 (d, J=8.0 Hz, 1H), 7.79 (d, J=9.2 Hz, 1H), 7.66-7.30 (m, 7H), 6.57 (s, 1H), 4.17-3.95 (m, 9H), 3.11-2.91 (m, 4H), 2.42-2.31 (m, 6H), 1.93-1.85 (m, 2H).

### Example 243: (S)-8-(2,2′-dichloro-3′-(6-methoxy-5-((((5-oxopyrrolidin-2-yl)-methyl)-amino)-methyl)pyridin-2-yl)-[1,1′-biphenyl]-3-yl)-3-((((3-hydroxyoxetan-3-yl)methyl)-amino)-methyl)-4H-pyrido[1,2-a]-pyrimidin-4-one

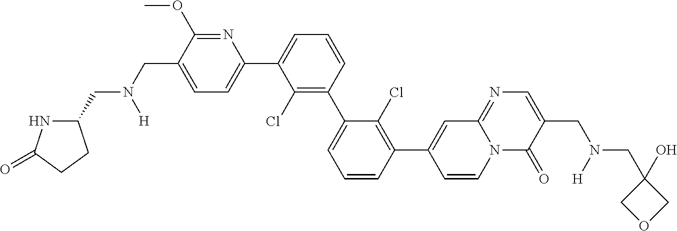 [SMILES: COc1nc(-c2cccc(-c3cccc(-c4ccn5c(=O)c(CNCC6(O)COC6)cnc5c4)c3Cl)c2Cl)ccc1CNC[C@@H]1CCC(=O)N1]

Penultimate intermediate was prepared in the same manner as described for Example 223, utilizing intermediate (S)-tert-butyl ((6-(2,2′-dichloro-3′-(3-formyl-4-oxo-4H-pyrido[1,2-a]pyrimidin-8-yl)-[1,1′-biphenyl]-3-yl)-2-methoxypyridin-3-yl)methyl)((5-oxopyrrolidin-2-yl)methyl)carbamate. 4,4,5,5,5-pentafluoropentan-1-amine hydrochloride was replaced by 3-(aminomethyl)oxetan-3-ol hydrochloride.

tert-butyl N-[[6-[2-chloro-3-[2-chloro-3-[3-[[2-(1,1-dioxo-1,4-thiazinan-4-yl)ethylamino]methyl]-4-oxo-pyrido[1,2-a]pyrimidin-8-yl]phenyl]phenyl]-2-methoxy-3-pyridyl]methyl]-N-[[(2S)-5-oxopyrrolidin-2-yl]methyl]carbamate (25 mg, 0.03 mmol) was dissolved in dioxane (3 mL). HCl solution (0.3 mL, 0.3 mmol, 4 M HCl in dioxane) was added and the reaction was stirred for 3 hours at room temperature. Solvent was evaporated and crude product was purified by reverse phase HPLC to afford 8-[2-chloro-3-[2-chloro-3-[6-methoxy-5-[[[(2S)-5-oxopyrrolidin-2-yl]methylamino]methyl]-2-pyridyl]phenyl]phenyl]-3-[[(3-hydroxyoxetan-3-yl)methylamino]methyl]pyrido[1,2-a]pyrimidin-4-one (bis-formic acid salt) as a white solid (2 mg, 9.1% yield). m/z: 716 [M+H]⁺ observed. ¹H NMR (400 MHz, CD₃OD) δ9.15 (d, J=7.6 Hz, 1H), 8.51 (s, 2H), 8.45 (s, 1H), 7.81 (s, 1H), 7.75 (dd, J=7.6 Hz, J=2.4 Hz, 1H), 7.66-7.51 (m, 6H), 7.40 (d, J=7.6 Hz, 1H), 7.24 (d, J=7.6 Hz, 1H), 4.09 (m, 2H), 4.02 (m, 3H), 3.91-3.84 (m, 3H), 3.67-3.60 (m, 4H), 3.08-3.00 (m, 2H), 2.80-2.72 (m, 2H), 2.34-2.25 (m, 3H), 1.86-1.76 (m, 1H).

### Example 244: (S)-8-(2,2′-dichloro-3′-(6-methoxy-5-((((5-oxopyrrolidin-2-yl)methyl)amino)-methyl)-pyridin-2-yl)-[1,1′-biphenyl]-3-yl)-3-((((3-(hydroxymethyl)oxetan-3-yl)-methyl)-amino)-methyl)-4H-pyrido[1,2-a]pyrimidin-4-one

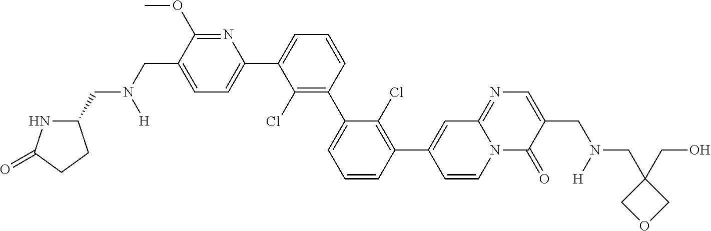 [SMILES: COc1nc(-c2cccc(-c3cccc(-c4ccn5c(=O)c(CNCC6(CO)COC6)cnc5c4)c3Cl)c2Cl)ccc1CNC[C@@H]1CCC(=O)N1]

Penultimate intermediate was prepared in the same manner as described for Example 223, utilizing intermediate (S)-tert-butyl ((6-(2,2′-dichloro-3′-(3-formyl-4-oxo-4H-pyrido[1,2-a]pyrimidin-8-yl)-[1,1′-biphenyl]-3-yl)-2-methoxypyridin-3-yl)methyl)((5-oxopyrrolidin-2-yl)methyl)carbamate. 4,4,5,5,5-pentafluoropentan-1-amine hydrochloride was replaced by (3-(aminomethyl)oxetan-3-yl)methanol hydrochloride.

tert-butyl N-[[6-[2-chloro-3-[2-chloro-3-[3-[[[3-(hydroxymethyl)oxetan-3-yl]methylamino]methyl]-4-oxo-pyrido[1,2-a]pyrimidin-8-yl]phenyl]phenyl]-2-methoxy-3-pyridyl]methyl]-N-[[(2S)-5-oxopyrrolidin-2-yl]methyl]carbamate (25 mg, 0.03 mmol) was dissolved in dioxane (3 mL). HCl solution (0.3 mL, 0.3 mmol, 4 M HCl in dioxane) was added and the reaction was stirred for 3 hours at room temperature. Solvent was evaporated and crude product was purified by reverse phase HPLC to afford 8-[2-chloro-3-[2-chloro-3-[6-methoxy-5-[[[(2S)-5-oxopyrrolidin-2-yl]methylamino]methyl]-2-pyridyl]phenyl]phenyl]-3-[[[3-(hydroxymethyl)oxetan-3-yl]methylamino]methyl]pyrido[1,2-a]pyrimidin-4-one (bis-formic acid salt) as a white solid (2 mg, 9.1% yield). m/z: 730 [M+H]⁺ observed. ¹H NMR (400 MHz, CD₃OD) δ9.16 (d, J=7.6 Hz, 1H), 8.48 (s, 3H), 7.84 (d, J=1.6 Hz, 1H), 7.81 (d, J=7.6 Hz, 1H), 7.67-7.58 (m, 4H), 7.54-7.49 (m, 2H), 7.40 (dd, J=7.6 Hz, J=1.6 Hz, 1H), 7.27 (d, J=7.6 Hz, 1H), 4.21 (m, 2H), 3.98 (s, 3H), 3.95-3.85 (m, 3H), 3.73-3.58 (m, 6H), 3.20 (s, 2H), 2.86-2.77 (m, 2H), 2.37-2.27 (m, 3H), 1.88-1.78 (m, 1H).

### Example 245: 8-(2,2′-dichloro-3′-(6-methoxy-5-(((((S)-5-oxopyrrolidin-2-yl)-methyl)amino)-methyl)-pyridin-2-yl)-[1,1′-biphenyl]-3-yl)-3-(((((1R,2R)-2-(hydroxymethyl)-cyclopropyl)methyl)amino)-methyl)-4H-pyrido[1,2-a]pyrimidin-4-one

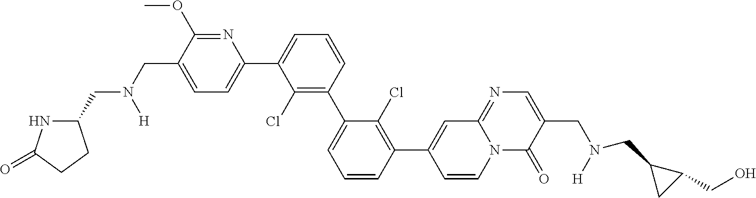 [SMILES: COc1nc(-c2cccc(-c3cccc(-c4ccn5c(=O)c(CNC[C@@H]6C[C@H]6CO)cnc5c4)c3Cl)c2Cl)ccc1CNC[C@@H]1CCC(=O)N1]

Penultimate intermediate was prepared in the same manner as described for Example 223, utilizing intermediate (S)-tert-butyl ((6-(2,2′-dichloro-3′-(3-formyl-4-oxo-4H-pyrido[1,2-a]pyrimidin-8-yl)-[1,1′-biphenyl]-3-yl)-2-methoxypyridin-3-yl)methyl)((5-oxopyrrolidin-2-yl)methyl)carbamate. 4,4,5,5,5-pentafluoropentan-1-amine hydrochloride was replaced by rac-((1R,2R)-2-(aminomethyl)cyclopropyl)methanol hydrochloride.

tert-butyl N-[[6-[2-chloro-3-[2-chloro-3-[4-oxo-3-[[[rac-(1R,2R)-2-(hydroxymethyl)cyclopropyl]methylamino]methyl]pyrido[1,2-a]pyrimidin-8-yl]phenyl]phenyl]-2-methoxy-3-pyridyl]methyl]-N-[[(2S)-5-oxopyrrolidin-2-yl]methyl]carbamate (25 mg, 0.03 mmol) was dissolved in dioxane (3 mL). HCl solution (0.3 mL, 0.3 mmol, 4 M HCl in dioxane) was added and the reaction was stirred for 3 hours at room temperature. Solvent was evaporated and crude product was purified by reverse phase HPLC to afford 8-[2-chloro-3-[2-chloro-3-[6-methoxy-5-[[[(2S)-5-oxopyrrolidin-2-yl]methylamino]methyl]-2-pyridyl]phenyl]phenyl]-3-[[[rac-(1R,2R)-2-(hydroxymethyl)cyclopropyl]methylamino]methyl]pyrido[1,2-a]pyrimidin-4-one (bis-formic acid salt) as a white solid (1 mg, 5% yield). m/z: 714 [M+H]⁺ observed. ¹H NMR (400 MHz, CD₃OD) δ9.19 (d, J=7.2 Hz, 1H), 8.51 (s, 3H), 7.85 (d, J=1.6 Hz, 1H), 7.75 (d, J=7.2 Hz, 1H), 7.67-7.59 (m, 4H), 7.54-7.49 (m, 2H), 7.40 (dd, J=7.2 Hz, J=1.6 Hz, 1H), 7.25 (d, J=7.2 Hz, 1H), 4.63 (s, 1H), 4.29 (s, 2H), 4.02 (s, 3H), 3.90-3.83 (m, 3H), 3.63-3.58 (m, 1H), 3.15-2.95 (m, 2H), 2.60-2.48 (m, 2H), 2.38-2.27 (m, 3H), 1.88-1.68 (m, 1H), 1.18-1.05 (m, 1H), 0.70-0.62 (m, 1H) and 0.09 (broad s, 1H).

### Example 246: (S)-8-(2,2′-dichloro-3′-(6-methoxy-5-((((5-oxopyrrolidin-2-yl)-methyl)amino)-methyl)pyridin-2-yl)-[1,1′-biphenyl]-3-yl)-3-((((3-methyloxetan-3-yl)-methyl)-amino)-methyl)-4H-pyrido[1,2-a]pyrimidin-4-one

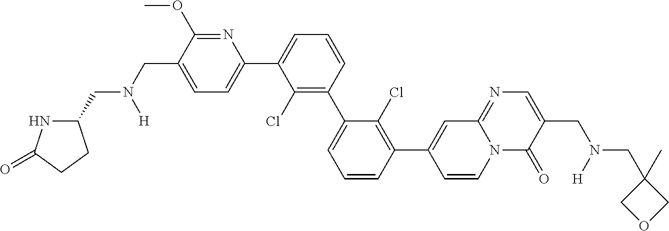 [SMILES: COc1nc(-c2cccc(-c3cccc(-c4ccn5c(=O)c(CNCC6(C)COC6)cnc5c4)c3Cl)c2Cl)ccc1CNC[C@@H]1CCC(=O)N1]

Penultimate intermediate was prepared in the same manner as described for Example 223, utilizing intermediate (S)-tert-butyl ((6-(2,2′-dichloro-3′-(3-formyl-4-oxo-4H-pyrido[1,2-a]pyrimidin-8-yl)-[1,1′-biphenyl]-3-yl)-2-methoxypyridin-3-yl)methyl)((5-oxopyrrolidin-2-yl)methyl)carbamate. 4,4,5,5,5-pentafluoropentan-1-amine hydrochloride was replaced by (3-methyloxetan-3-yl)methanamine hydrochloride.

tert-butyl N-[[6-[2-chloro-3-[2-chloro-3-[3-[[(3-methyloxetan-3-yl)methylamino]methyl]-4-oxo-pyrido[1,2-a]pyrimidin-8-yl]phenyl]phenyl]-2-methoxy-3-pyridyl]methyl]-N-[[(2S)-5-oxopyrrolidin-2-yl]methyl]carbamate (25 mg, 0.03 mmol) was dissolved in dioxane (3 mL). HCl solution (0.3 mL, 0.3 mmol, 4 M HCl in dioxane) was added and the reaction was stirred for 3 hours at room temperature. Solvent was evaporated and crude product was purified by reverse phase HPLC to afford 8-[2-chloro-3-[2-chloro-3-[6-methoxy-5-[[[(2S)-5-oxopyrrolidin-2-yl]methylamino]methyl]-2-pyridyl]phenyl]phenyl]-3-[[(3-methyloxetan-3-yl)methylamino]-methyl]pyrido[1,2-a]pyrimidin-4-one (bis-formic acid salt) as a white solid (3 mg, 13.7% yield). m/z: 714 [M+H]⁺ observed. ¹H NMR (400 MHz, CD₃OD) δ9.09 (d, J=1.2 Hz, 1H), 8.38 (brs, 1H), 8.13 (dd, J=9.2 Hz, J=1.2 Hz, 1H), 7.84 (d, J=8.0 Hz, 1H), 7.79 (d, J=9.2 Hz, 1H), 7.66-7.30 (m, 7H), 6.57 (s, 1H), 4.17-3.95 (m, 9H), 3.11-2.91 (m, 4H), 2.42-2.31 (m, 6H), 1.93-1.85 (m, 2H).

### Example 264: (S)-8-(2,2′-dichloro-3′-(6-methoxy-5-((((5-oxopyrrolidin-2-yl)methyl)-amino)-methyl)-pyridin-2-yl)-[1,1′-biphenyl]-3-yl)-3-(((2-(3-oxopiperazin-1-yl)ethyl)amino)methyl)-4H-pyrido[1,2-a]pyrimidin-4-one

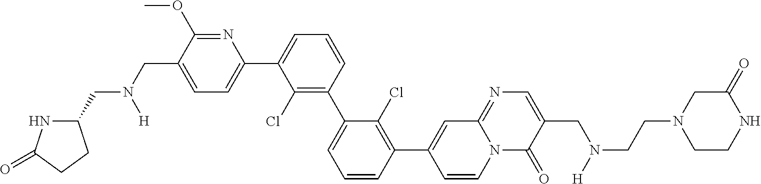 [SMILES: COc1nc(-c2cccc(-c3cccc(-c4ccn5c(=O)c(CNCCN6CCNC(=O)C6)cnc5c4)c3Cl)c2Cl)ccc1CNC[C@@H]1CCC(=O)N1]

Penultimate intermediate was prepared in the same manner as described for Example 223, utilizing intermediate (S)-tert-butyl ((6-(2,2′-dichloro-3′-(3-formyl-4-oxo-4H-pyrido[1,2-a]pyrimidin-8-yl)-[1,1′-biphenyl]-3-yl)-2-methoxypyridin-3-yl)methyl)((5-oxopyrrolidin-2-yl)methyl)carbamate. 4,4,5,5,5-pentafluoropentan-1-amine hydrochloride was replaced by 4-(2-aminoethyl)piperazin-2-one hydrochloride.

tert-butyl N-[[6-[2-chloro-3-[2-chloro-3-[4-oxo-3-[[2-(3-oxopiperazin-1-yl)ethylamino]methyl]pyrido[1,2-a]pyrimidin-8-yl]phenyl]phenyl]-2-methoxy-3-pyridyl]methyl]-N-[[(2S)-5-oxopyrrolidin-2-yl]methyl]carbamate (26 mg, 0.03 mmol) was dissolved in dioxane (3 mL). HCl solution (0.1 mL, 0.4 mmol, 4 M HCl in dioxane) was added and the reaction was stirred for 3 hours at room temperature. Solvent was evaporated and crude product was purified by reverse phase HPLC to afford 8-[2-chloro-3-[2-chloro-3-[6-methoxy-5-[[[(2S)-5-oxopyrrolidin-2-yl]methylamino]methyl]-2-pyridyl]phenyl]phenyl]-3-[[2-(3-oxopiperazin-1-yl)ethylamino]methyl]pyrido[1,2-a]pyrimidin-4-one (bis-formic acid salt) as a white solid (2 mg, 8.7% yield). m/z: 756 [M+H]⁺ observed. ¹H NMR (400 MHz, CD₃OD) δ9.15 (d, J=7.2 Hz, 1H), 8.50 (s, 1H), 7.86 (s, 1H), 7.88 (d, J=7.2 Hz, 1H), 7.67-7.37 (m, 7H), 7.28 (d, J=7.2 Hz, 1H), 4.30 (s, 2H), 4.05-3.90 (m, 6H), 3.38-3.17 (m, 6H), 2.91-2.77 (m, 6H), 2.38-3.30 (m, 3H), 1.90-1.80 (m, 1H).

### Example 266: (S)-8-(2,2′-dichloro-3′-(6-methoxy-5-((((5-oxopyrrolidin-2-yl)methyl)-amino)methyl)-pyridin-2-yl)-[1,1′-biphenyl]-3-yl)-3-(((2-(1,1-dioxidothiomorpholino)-ethyl)-amino)-methyl)-4H-pyrido[1,2-a]pyrimidin-4-one

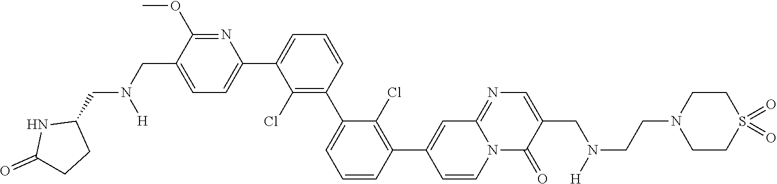 [SMILES: COc1nc(-c2cccc(-c3cccc(-c4ccn5c(=O)c(CNCCN6CCS(=O)(=O)CC6)cnc5c4)c3Cl)c2Cl)ccc1CNC[C@@H]1CCC(=O)N1]

Penultimate intermediate was prepared in the same manner as described for Example 223, utilizing intermediate (S)-tert-butyl ((6-(2,2′-dichloro-3′-(3-formyl-4-oxo-4H-pyrido[1,2-a]pyrimidin-8-yl)-[1,1′-biphenyl]-3-yl)-2-methoxypyridin-3-yl)methyl)((5-oxopyrrolidin-2-yl)methyl)carbamate. 4,4,5,5,5-pentafluoropentan-1-amine hydrochloride was replaced by 4-(2-aminoethyl)thiomorpholine 1,1-dioxide hydrochloride.

tert-butyl N-[[6-[2-chloro-3-[2-chloro-3-[3-[[2-(1,1-dioxo-1,4-thiazinan-4-yl)ethylamino]methyl]-4-oxo-pyrido[1,2-a]pyrimidin-8-yl]phenyl]phenyl]-2-methoxy-3-pyridyl]methyl]-N-[[(2S)-5-oxopyrrolidin-2-yl]methyl]carbamate (27 mg, 0.03 mmol) was dissolved in dioxane (3 mL). HCl solution (0.1 mL, 0.4 mmol, 4 M HCl in dioxane) was added and the reaction was stirred for 3 hours at room temperature. Solvent was evaporated and crude product was purified by reverse phase HPLC to afford 8-[2-chloro-3-[2-chloro-3-[6-methoxy-5-[[[(2S)-5-oxopyrrolidin-2-yl]methylamino]methyl]-2-pyridyl]phenyl]phenyl]-3-[[2-(1,1-dioxo-1,4-thiazinan-4-yl)ethylamino]methyl]pyrido[1,2-a]pyrimidin-4-one (bis-formic acid salt) as a white solid (1.5 mg, 6.3% yield). m/z: 791 [M+H]⁺ observed. ¹H NMR (400 MHz, CD₃OD) δ9.16 (d, J=7.6 Hz, 1H), 8.53 (s, 1H), 8.45 (s, 1H), 7.82 (d, J=1.6 Hz, 1H), 7.73 (d, J=7.6 Hz, 1H), 7.66-7.49 (m, 6H), 7.39 (dd, J=7.6 Hz, J=1.6 Hz, 1H), 7.24 (d, J=7.6 Hz, 1H), 4.07 (s, 2H), 4.01 (s, 3H), 3.87-3.79 (m, 3H), 3.14 (broad s, 4H), 2.99 (broad s, 4H), 2.94 (broad s, 2H), 2.79-2.65 (m, 4H), 2.35-2.24 (m, 3H), 1.85-1.75 (m, 1H).

### Example 358: methyl (S)-3-((((8-(2,2′-dichloro-3′-(6-methoxy-5-((((5-oxopyrrolidin-2-yl)-methyl)-amino)-methyl)-pyridin-2-yl)-[1,1′-biphenyl]-3-yl)-4-oxo-4H-pyrido[1,2-a]pyrimidin-3-yl)-methyl)-amino)-methyl)oxetane-3-carboxylate

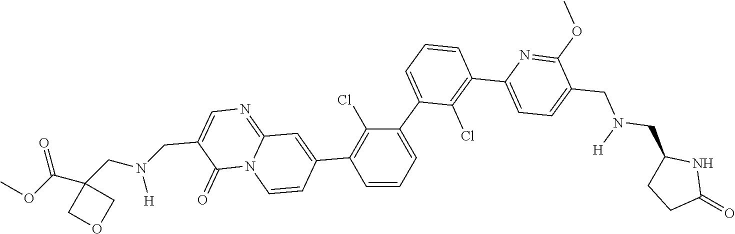 [SMILES: COC(=O)C1(CNCc2cnc3cc(-c4cccc(-c5cccc(-c6ccc(CNC[C@@H]7CCC(=O)N7)c(OC)n6)c5Cl)c4Cl)ccn3c2=O)COC1]

tert-butyl N-[[6-[2-chloro-3-[2-chloro-3-(3-formyl-4-oxo-pyrido[1,2-a]pyrimidin-8-yl)phenyl]phenyl]-2-methoxy-3-pyridyl]methyl]-N-[[(2S)-5-oxopyrrolidin-2-yl]methyl]carbamate (30 mg, 0.041 mmol), methyl 3-(aminomethyl)oxetane-3-carboxylate (9 mg, 0.062 mmol), and acetic acid (10 mg, 0.16 mmol) were dissolved in in dioxane/methanol (1:1, 3 mL). 3 Å molecular sieves (500 mg) were added and reaction was stirred overnight at 90° C. Reaction was cooled to rt and sodium triacetoxyborohydride (90 mg, 0.42 mmol) was added, then the mixture was stirred for 1 h at room temperature. Reaction was diluted with water (10 mL), extracted with dichloromethane (2×20 mL), and the combined organic layer was washed with brine (30 mL), dried over sodium sulfate (10 g), filtered, and concentrated to give crude product (45 mg) as an orange solid. Material was used for the next reaction without further purification.

The orange solid from reaction above (45 mg, 0.05 mmol) was dissolved in dioxane (3 mL). HCl solution (0.3 mL, 0.3 mmol, 4 M HCl in dioxane) (0.5 mL, 2 mmol, 4 M HCl in dioxane) was added and the reaction was stirred for 3 hours at room temperature. Solvent was evaporated and crude product was purified by reverse phase HPLC to afford methyl 3-[[[8-[2-chloro-3-[2-chloro-3-[6-methoxy-5-[[[(2S)-5-oxopyrrolidin-2-yl]methylamino]methyl]-2-pyridyl]phenyl]phenyl]-4-oxo-pyrido[1,2-a]pyrimidin-3-yl]methylamino]methyl]oxetane-3-carboxylate (bis-formic acid salt) as a white solid (10 mg, 21.3% yield). m/z: 758 [M+H]⁺ observed. ¹H NMR (400 MHz, CD₃OD) δ9.11 (d, J=7.6 Hz, 1H), 8.50 (s, 1H), 8.41 (s, 1H), 7.79-7.77 (m, 2H), 7.66-7.48 (m, 6H), 7.39 (dd, J=7.6 Hz and J=1.6 Hz, 1H), 7.26 (d, J=7.6 Hz, 1H), 4.02-3.74 (m, 11H), 3.06-2.82 (m, 4H), 2.38-2.29 (m, 3H), 2.04 (s, 3H), 1.86-1.81 (m, 1H).

### Example 359: 8-(2,2′-dichloro-3′-(6-methoxy-5-(((((S)-5-oxopyrrolidin-2-yl)methyl)-amino)-methyl)-pyridin-2-yl)-[1,1′-biphenyl]-3-yl)-3-((((3,3-dimethyl-5-oxopyrrolidin-2-yl)methyl)amino)methyl)-4H-pyrido[1,2-a]pyrimidin-4-one

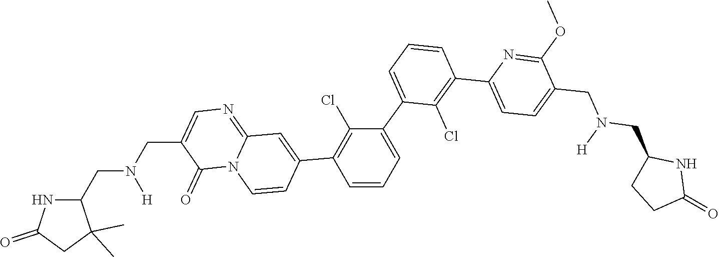 [SMILES: COc1nc(-c2cccc(-c3cccc(-c4ccn5c(=O)c(CNCC6NC(=O)CC6(C)C)cnc5c4)c3Cl)c2Cl)ccc1CNC[C@@H]1CCC(=O)N1]

tert-butyl N-[[6-[2-chloro-3-[2-chloro-3-(3-formyl-4-oxo-pyrido[1,2-a]pyrimidin-8-yl)phenyl]phenyl]-2-methoxy-3-pyridyl]methyl]-N-[[(2S)-5-oxopyrrolidin-2-yl]methyl]carbamate (30 mg, 0.041 mmol), 5-(aminomethyl)-4,4-dimethyl-pyrrolidin-2-one (10 mg, 0.07 mmol) and acetic acid were dissolved in in dioxane/methanol (1:1, 3 mL). 3 Å molecular sieves (500 mg) were added and reaction was stirred overnight at 65° C. Reaction was cooled to rt and sodium triacetoxyborohydride (90 mg, 0.42 mmol) was added, then the mixture was stirred for 1 h at room temperature. Reaction was diluted with water (10 mL), extracted with dichloromethane (2×20 mL), and the combined organic layer was washed with brine (30 mL), dried over sodium sulfate (10 g), filtered, and concentrated to give crude product (45 mg) as an orange solid. Material was used for the next reaction without further purification.

The orange solid from reaction above (45 mg, 0.05 mmol) was dissolved in dioxane (3 mL). HCl solution (0.3 mL, 0.3 mmol, 4 M HCl in dioxane) (0.5 mL, 2 mmol, 4 M HCl in dioxane) was added and the reaction was stirred for 3 hours at room temperature. Solvent was evaporated and crude product was purified by reverse phase HPLC to afford 8-(2,2′-dichloro-3′-(6-methoxy-5-(((((S)-5-oxopyrrolidin-2-yl)methyl)-amino)-methyl)-pyridin-2-yl)-[1,1′-biphenyl]-3-yl)-3-((((3,3-dimethyl-5-oxopyrrolidin-2-yl)methyl)amino)methyl)-4H-pyrido[1,2-a]pyrimidin-4-one (bis-formic acid salt) as a brown solid (5 mg, 17% yield). m/z: 755 [M+H]⁺ observed. ¹H NMR (400 MHz, CD₃OD) δ9.14 (d, J=7.6 Hz, 1H), 8.48 (s, 1H), 8.44 (s, 1H), 7.78-7.75 (m, 2H), 7.66-7.50 (m, 6H), 7.39 (dd, J=7.6 Hz, J=1.6 Hz, 1H), 7.26 (d, J=7.6 Hz, 1H), 4.02-3.86 (m, 8H), 3.48 (dd, J=9.6 Hz, J=3.2 Hz, 1H), 2.93 (dd, J=9.6 Hz, J=3.2 Hz, 1H),2.84-2.72 (m, 3H), 2.36-2.27 (m, 3H), 2.18 (s, 2H), 1.85-1.80 (m, 1H), 1.20 (s, 3H) and 1.05 (s, 3H).

### Example 368: 8-(2,2′-dichloro-3′-(6-methoxy-5-(((((S)-5-oxopyrrolidin-2-yl)-methyl)-amino)-methyl)-pyridin-2-yl)-[1,1′-biphenyl]-3-yl)-3-((((2-methyl-5-oxopyrrolidin-2-yl)methyl)-amino)-methyl)-4H-pyrido[1,2-a]pyrimidin-4-one

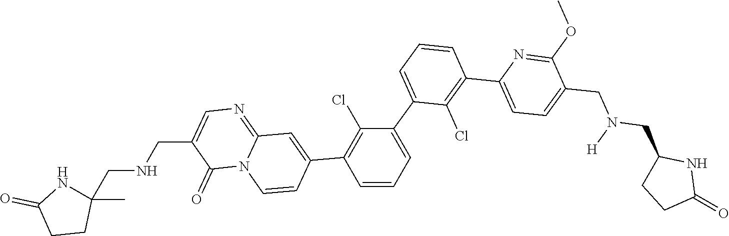 [SMILES: COc1nc(-c2cccc(-c3cccc(-c4ccn5c(=O)c(CNCC6(C)CCC(=O)N6)cnc5c4)c3Cl)c2Cl)ccc1CNC[C@@H]1CCC(=O)N1]

tert-butyl N-[[6-[2-chloro-3-[2-chloro-3-(3-formyl-4-oxo-pyrido[1,2-a]pyrimidin-8-yl)phenyl]phenyl]-2-methoxy-3-pyridyl]methyl]-N-[[(2S)-5-oxopyrrolidin-2-yl]methyl]carbamate (30 mg, 0.041 mmol), 5-(aminomethyl)-5-methyl-pyrrolidin-2-one (8.5 mg, 0.065 mmol) and acetic acid (10 mg, 0.16 mmol) were dissolved in in dioxane/methanol (1:1, 3 mL). 3 Å molecular sieves (500 mg) were added and reaction was stirred overnight at 65° C. Reaction was cooled to rt and sodium triacetoxyborohydride (90 mg, 0.42 mmol) was added, then the mixture was stirred for 1 h at room temperature. Reaction was diluted with water (10 mL), extracted with dichloromethane (2×20 mL), and the combined organic layer was washed with brine (30 mL), dried over sodium sulfate (10 g), filtered, and concentrated to give crude product (45 mg) as an orange solid. Material was used for the next reaction without further purification.

The orange solid from reaction above (45 mg, 0.05 mmol) was dissolved in dioxane (3 mL). HCl solution (0.3 mL, 0.3 mmol, 4 M HCl in dioxane) (0.5 mL, 2 mmol, 4 M HCl in dioxane) was added and the reaction was stirred for 3 hours at room temperature. Solvent was evaporated and crude product was purified by reverse phase HPLC to afford 8-(2,2′-dichloro-3′-(6-methoxy-5-(((((S)-5-oxopyrrolidin-2-yl)-methyl)-amino)-methyl)-pyridin-2-yl)-[1,1′-biphenyl]-3-yl)-3-((((2-methyl-5-oxopyrrolidin-2-yl)methyl)-amino)-methyl)-4H-pyrido[1,2-a]pyrimidin-4-one (3.5 mg, 10% yield) as an off white solid (bis-formic acid salt). m/z: 741 [M+H]⁺ observed. ¹H NMR (400 MHz, CD₃OD) δ9.14 (d, J=7.6 Hz, 1H), 8.44 (s, 1H), 8.42 (s, 2H), 7.81-7.79 (broad s, 2H), 7.66-7.50 (m, 6H), 7.41 (dd, J=7.6 Hz, J=1.6 Hz, 1H), 7.29 (d, J=7.6 Hz, 1H), 4.08-3.90 (m, 8H), 2.96-2.84 (m, 4H), 2.44-2.30 (m, 5H), 2.20-2.13 (m, 1H), 1.93-1.83 (m, 2H) and 1.30 (s, 3H).

### Example 384: methyl (S)-3-(((8-(2,2′-dichloro-3′-(6-methoxy-5-((((5-oxopyrrolidin-2-yl)-methyl)-amino)-methyl)-pyridin-2-yl)-[1,1′-biphenyl]-3-yl)-4-oxo-4H-pyrido[1,2-a]pyrimidin-3-yl)-methyl)-amino)-2,2-dimethylpropanoate

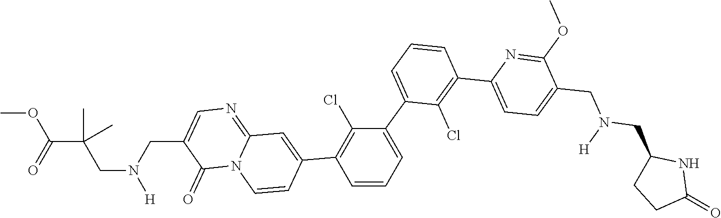 [SMILES: COC(=O)C(C)(C)CNCc1cnc2cc(-c3cccc(-c4cccc(-c5ccc(CNC[C@@H]6CCC(=O)N6)c(OC)n5)c4Cl)c3Cl)ccn2c1=O]

tert-butyl N-[[6-[2-chloro-3-[2-chloro-3-(3-formyl-4-oxo-pyrido[1,2-a]pyrimidin-8-yl)phenyl]phenyl]-2-methoxy-3-pyridyl]methyl]-N-[[(2S)-5-oxopyrrolidin-2-yl]methyl]carbamate (30 mg, 0.041 mmol), methyl 2-amino-2-methyl-propanoate (8 mg, 0.065 mmol) and acetic acid (10 mg, 0.16 mmol) were dissolved in in dioxane/methanol (1:1, 3 mL). 3 Å molecular sieves (500 mg) were added and reaction was stirred overnight at 80° C. Reaction was cooled to rt and sodium triacetoxyborohydride (90 mg, 0.42 mmol) was added, then the mixture was stirred for 1 h at room temperature. Reaction was diluted with water (10 mL), extracted with dichloromethane (2×20 mL), and the combined organic layer was washed with brine (30 mL), dried over sodium sulfate (10 g), filtered, and concentrated to give crude product (45 mg) as an orange solid. Material was used for the next reaction without further purification.

The orange solid from reaction above (45 mg, 0.05 mmol) was dissolved in dioxane (3 mL). HCl solution (0.3 mL, 0.3 mmol, 4 M HCl in dioxane) (0.5 mL, 2 mmol, 4 M HCl in dioxane) was added and the reaction was stirred for 3 hours at room temperature. Solvent was evaporated and crude product was purified by reverse phase HPLC to afford (S)-3-(((8-(2,2′-dichloro-3′-(6-methoxy-5-((((5-oxopyrrolidin-2-yl)-methyl)-amino)-methyl)-pyridin-2-yl)-[1,1′-biphenyl]-3-yl)-4-oxo-4H-pyrido[1,2-a]pyrimidin-3-yl)-methyl)-amino)-2,2-dimethylpropanoate (bis-formic acid salt) as a white solid (15 mg, 66% yield). m/z: 744 [M+H]⁺ observed. ¹H NMR (400 MHz, CD₃OD) δ9.11 (d, J=7.6 Hz, 1H), 8.38 (s, 1H), 7.76-7.71 (m, 2H), 7.66-7.57 (m, 3H), 7.53-7.48 (m, 3H), 7.38 (dd, J=7.6 Hz and J=1.6 Hz, 1H), 7.24 (d, J=7.6 Hz, 1H), 4.01 (s, 3H), 3.86-3.78 (m, 5H), 3.66 (s, 3H), 2.73-2.64 (m, 4H), 2.35-2.24 (m, 3H), 1.85-1.78 (m, 1H) and 1.19 (s, 6H).

### Example 213: 8-(2-chloro-3-(1-((3-chloro-6-methoxy-5-(((((S)-5-oxopyrrolidin-2-yl)methyl)amino)methyl)pyridin-2-yl)methyl)-2,3-dihydro-1H-inden-4-yl)phenyl)-3-(((((S)-5-oxopyrrolidin-2-yl)methyl)amino)methyl)-4H-pyrido[1,2-a]pyrimidin-4-one

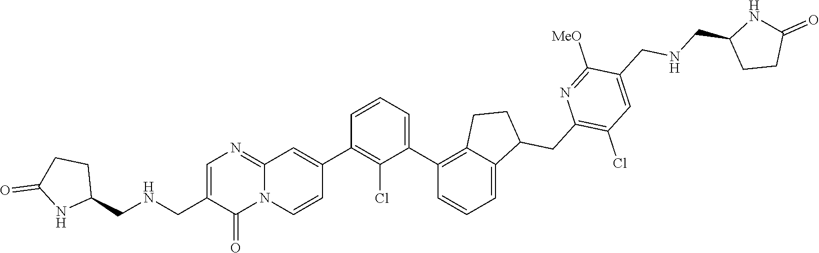 [SMILES: COc1nc(CC2CCc3c(-c4cccc(-c5ccn6c(=O)c(CNC[C@@H]7CCC(=O)N7)cnc6c5)c4Cl)cccc32)c(Cl)cc1CNC[C@@H]1CCC(=O)N1]

### 2-((4-bromo-2,3-dihydro-1H-inden-1-yl)methyl)-3-chloro-5-(1,3-dioxolan-2-yl)-6-methoxypyridine

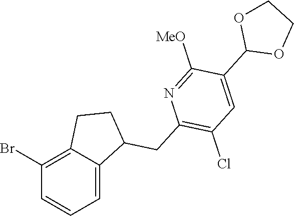 [SMILES: COc1nc(CC2CCc3c(Br)cccc32)c(Cl)cc1C1OCCO1]

To a mixture of (E)-2-((4-bromo-2,3-dihydro-1H-inden-1-ylidene)methyl)-3-chloro-5-(1,3-dioxolan-2-yl)-6-methoxypyridine (0.3 g, 0.71 mmol) in ethylene glycol (40 mL) was added sodium hydrogen carbonate (0.6 g, 7.10 mmol) and p-toluenesulfonyl hydrazide (1.32 g, 7.10 mmo). The mixture was stirred at 135° C. for 18 hours. Reaction was cooled to rt and added to water (50 mL), then the aqueous solution was extracted with EtOAc (3×50 ml). The combined organic layers were washed with saturated aqueous brine solution (40 mL), dried over anhydrous sodium sulfate, filtered, and concentrated under reduced pressure to give a residue. The crude sample was purified by normal phase SiO₂ chromatography (0-100% EtOAc/petroleum ether) to afford 2-((4-bromo-2,3-dihydro-1H-inden-1-yl)methyl)-3-chloro-5-(1,3-dioxolan-2-yl)-6-methoxypyridine as a yellow oil (0.42 g, 33% yield, 72% purity). ¹H NMR (400 MHz, DMSO-d₆): δ7.75 (s, 1H), 7.35 (d, J=7.6 Hz, 1H), 7.13-7.06 (m, 2H), 5.87 (s, 1H), 4.06-3.92 (m, 4H), 3.85-3.79 (m, 4H), 3.28 (m, 1H), 2.93-2.82 (m, 3H), 2.23-2.19 (m, 1H), 1.82 (m, 1H).

### 8-(2-chloro-3-(1-((3-chloro-5-(1,3-dioxolan-2-yl)-6-methoxypyridin-2-yl)methyl)-2,3-dihydro-1H-inden-4-yl)phenyl)-4-oxo-4H-pyrido[1,2-a]pyrimidine-3-carbaldehyde

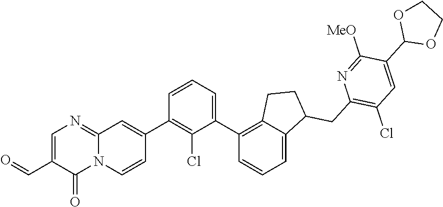 [SMILES: COc1nc(CC2CCc3c(-c4cccc(-c5ccn6c(=O)c(C=O)cnc6c5)c4Cl)cccc32)c(Cl)cc1C1OCCO1]

To a mixture of 2-((4-bromo-2,3-dihydro-1H-inden-1-yl)methyl)-3-chloro-5-(1,3-dioxolan-2-yl)-6-methoxypyridine (0.41 g, 0.99 mmol) in water/THF/dioxane (1:2:2, 7.5 mL) were added 8-(2-chloro-3-(4,4,5,5-tetramethyl-1,3,2-dioxaborolan-2-yl)phenyl)-4-oxo-4H-pyrido[1,2-a]pyrimidine-3-carbaldehyde (0.28 g, 0.66 mmol), [1,1′-bis(di-tert-butylphosphino)ferrocene]dichloropalladium(II) (0.04 g, 0.07 mmol), and potassium phosphate (0.14 g, 0.66 mmol). Reaction was stirred at 80° C. for 2 hours. The mixture was cooled to rt and diluted with water (30 mL), then extracted with EtOAc (3×30 ml). The combined organic layers were washed with saturated aqueous brine solution (40 mL), dried over anhydrous sodium sulfate, filtered, and concentrated under reduced pressure to give a residue. The crude sample was purified by normal phase SiO₂ chromatography (0-100% EtOAc/petroleum ether) to afford 8-(2-chloro-3-(1-((3-chloro-5-(1,3-dioxolan-2-yl)-6-methoxypyridin-2-yl)methyl)-2,3-dihydro-1H-inden-4-yl)phenyl)-4-oxo-4H-pyrido[1,2-a]pyrimidine-3-carbaldehyde as a yellow oil (0.3 g, 42% yield). ¹H NMR (400 MHz, DMSO-d₆): δ10.20 (s, 1H), 9.27 ((d, J=7.2 Hz, 1H), 8.79 (s, 1H), 8.04 (m, 1H), 7.82-7.58 (m, 5H), 7.26-7.13 (m, 3H), 5.89 (s, 1H), 4.06-3.80 (m, 8H), 3.38 (m, 3H), 2.99-2.95 (m, 1H), 2.18-2.15 (m, 1H), 1.80-1.76 (m, 1H).

### 8-(2-chloro-3-(1-((3-chloro-5-formyl-6-methoxypyridin-2-yl)methyl)-2,3-dihydro-1H-inden-4-yl)phenyl)-4-oxo-4H-pyrido[1,2-a]pyrimidine-3-carbaldehyde

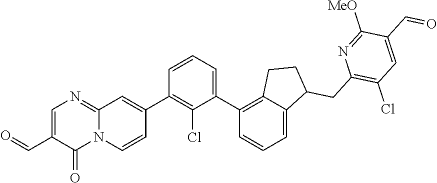 [SMILES: COc1nc(CC2CCc3c(-c4cccc(-c5ccn6c(=O)c(C=O)cnc6c5)c4Cl)cccc32)c(Cl)cc1C=O]

To a solution of 8-(2-chloro-3-(1-((3-chloro-5-(1,3-dioxolan-2-yl)-6-methoxypyridin-2-yl)methyl)-2,3-dihydro-1H-inden-4-yl)phenyl)-4-oxo-4H-pyrido[1,2-a]pyrimidine-3-carbaldehyde (0.3 g, 0.48 mol) in THE (5 mL) was added concentrated HCl (12 M, 2 mL) and the mixture was stirred at 25° C. for 1 hour. The solution was concentrated under reduced pressure, and the residue was poured into an ice-water solution, then treated with saturated aqueous sodium bicarbonate solution to pH=7. The aqueous solution was extracted with EtOAc(3×50 ml) and the combined organic layers were washed with saturated aqueous brine solution (40 mL), dried over anhydrous sodium sulfate, filtered and concentrated under reduced pressure to afford 8-(2-chloro-3-(1-((3-chloro-5-formyl-6-methoxypyridin-2-yl)methyl)-2,3-dihydro-1H-inden-4-yl)phenyl)-4-oxo-4H-pyrido[1,2-a]pyrimidine-3-carbaldehyde as yellow oil (0.3 g, crude, m/z: 584.0 [M+H]⁺ observed). Material was used for the next step without further purification.

### 8-(2-chloro-3-(1-((3-chloro-6-methoxy-5-(((((S)-5-oxopyrrolidin-2-yl)methyl)amino)methyl)pyridin-2-yl)methyl)-2,3-dihydro-1H-inden-4-yl)phenyl)-3-(((((S)-5-oxopyrrolidin-2-yl)methyl)amino)methyl)-4H-pyrido[1,2-a]pyrimidin-4-one

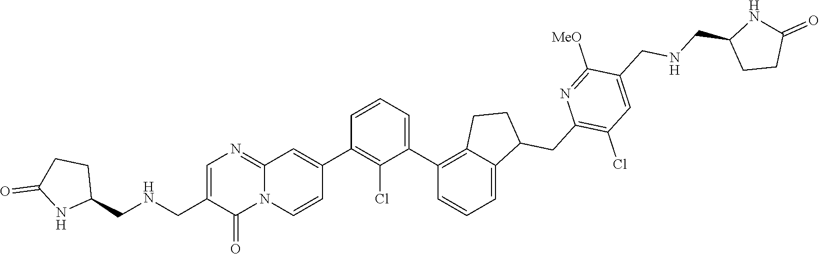 [SMILES: COc1nc(CC2CCc3c(-c4cccc(-c5ccn6c(=O)c(CNC[C@@H]7CCC(=O)N7)cnc6c5)c4Cl)cccc32)c(Cl)cc1CNC[C@@H]1CCC(=O)N1]

To a solution of 8-(2-chloro-3-(1-((3-chloro-5-formyl-6-methoxypyridin-2-yl)methyl)-2,3-dihydro-1H-inden-4-yl)phenyl)-4-oxo-4H-pyrido[1,2-a]pyrimidine-3-carbaldehyde (0.3 g, 0.51 mmol) in DCM/MeOH (2:1, 3 mL) were added (S)-5-(aminomethyl)pyrrolidin-2-one hydrochloride (0.46 g, 3.08 mmol), and sodium acetate (0.25 g, 3.08 mmol). The resulting mixture was stirred at 25° C. for 1 hour, then sodium cyanoborohydride (0.19 g, 3.08 mmol) was added. Reaction was further stirred at 25° C. for 2 hours, then the mixture was filtered and concentrated under reduced pressure to give a residue. Crude sample was purified by reverse phase HPLC to afford 8-(2-chloro-3-(1-((3-chloro-6-methoxy-5-(((((S)-5-oxopyrrolidin-2-yl)methyl)amino)methyl)pyridin-2-yl)methyl)-2,3-dihydro-1H-inden-4-yl)phenyl)-3-(((((S)-5-oxopyrrolidin-2-yl)methyl)amino)methyl)-4H-pyrido[1,2-a]pyrimidin-4-one as a white solid. (52.3 mg, 13% yield, m/z: 780.2 [M+H]⁺ observed). ¹H NMR (400 MHz, CD₃OD): δ9.14 (d, J=7.2 Hz, 1H), 8.43 (s, 1H), 7.77 (s, 1H), 7.68-7.66 (m, 1H), 7.57-7.43 (m, 3H), 7.46 (m, 1H), 7.24-7.18 (m, 2H), 7.09 (d, J=7.2 Hz, 1H), 3.96-3.84 (m, 10H), 3.38-3.36 (m, 1H), 3.04-3.01 (m, 1H), 2.79-2.65 (m, 6H), 2.38-2.20 (m, 7H), 1.91-1.80 (m, 3H). ¹H NMR (400 MHz, DMSO-d₆): 9.01 (d, J=7.2 Hz, 1H), 8.42 (s, 1H), 7.77 (s, 2H), 7.71-7.67 (m, 2H), 7.59-7.56 (m, 2H), 7.51-7.49 (m, 2H), 7.27-7.23 (m, 2H), 7.13 (d, J=6.8 Hz, 1H), 3.97-3.64 (m, 10H), 2.96-2.90 (m, 1H), 2.70-2.68 (m, 2H), 2.54 (m, 6H), 2.23-2.03 (m, 9H), 1.81-1.65 (m, 3H).

### Example 214: 8-[2-chloro-3-[2-chloro-3-[6-methoxy-5-[[[(2S)-5-oxopyrrolidin-2-yl]methylamino]methyl]-2-pyridyl]phenyl]phenyl]-3-[[2-[(2S)-5-oxopyrrolidin-2-yl]ethylamino]methyl]pyrido[1,2-a]pyrimidin-4-one

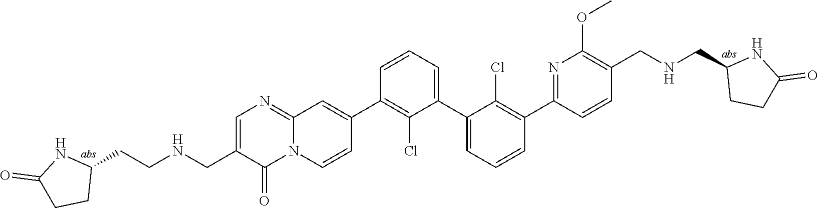 [SMILES: COc1nc(-c2cccc(-c3cccc(-c4ccn5c(=O)c(CNCC[C@@H]6CCC(=O)N6)cnc5c4)c3Cl)c2Cl)ccc1CNC[C@@H]1CCC(=O)N1]

### tert-Butyl N-[[6-[2-chloro-3-[2-chloro-3-[4-oxo-3-[[2-[(2S)-5-oxopyrrolidin-2-yl]ethylamino]methyl]pyrido[1,2-a]pyrimidin-8-yl]phenyl]phenyl]-2-methoxy-3-pyridyl]methyl]-N-[[(2S)-5-oxopyrrolidin-2-yl]methyl]carbamate

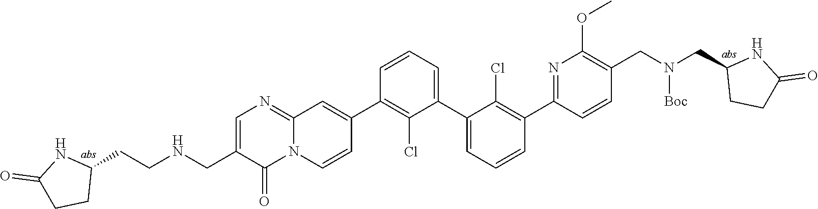 [SMILES: COc1nc(-c2cccc(-c3cccc(-c4ccn5c(=O)c(CNCC[C@@H]6CCC(=O)N6)cnc5c4)c3Cl)c2Cl)ccc1CN(C[C@@H]1CCC(=O)N1)C(=O)OC(C)(C)C]

A mixture of tert-butyl N-[[6-[2-chloro-3-[2-chloro-3-(3-formyl-4-oxo-pyrido[1,2-a]pyrimidin-8-yl)phenyl]phenyl]-2-methoxy-3-pyridyl]methyl]-N-[[(2S)-5-oxopyrrolidin-2-yl]methyl]carbamate (30 mg, 0.04 mmol), (5S)-5-(2-aminoethyl)pyrrolidin-2-one (11 mg, 0.08 mmol) and acetic acid (2 uL, 2.5 mg, 0.04 mmol) in MeOH/THF (2:1) was stirred for 2 hours at rt. NaBH₃CN (5 mg, 0.08 mmol) was added, the mixture was stirred for 2 hours further, quenched with water, and directly purified by reversed phase chromatography to give tert-butyl N-[[6-[2-chloro-3-[2-chloro-3-[4-oxo-3-[[2-[(2S)-5-oxopyrrolidin-2-yl]ethylamino]methyl]pyrido[1,2-a]pyrimidin-8-yl]phenyl]phenyl]-2-methoxy-3-pyridyl]methyl]-N-[[(2S)-5-oxopyrrolidin-2-yl]methyl]carbamate (30 mg, 86.7%). MS: m/z found 840.3 [M+H]⁺.

### 8-[2-Chloro-3-[2-chloro-3-[6-methoxy-5-[[[(2S)-5-oxopyrrolidin-2-yl]methylamino]methyl]-2-pyridyl]phenyl]phenyl]-3-[[2-[(2S)-5-oxopyrrolidin-2-yl]ethylamino]methyl]pyrido[1,2-a]pyrimidin-4-one

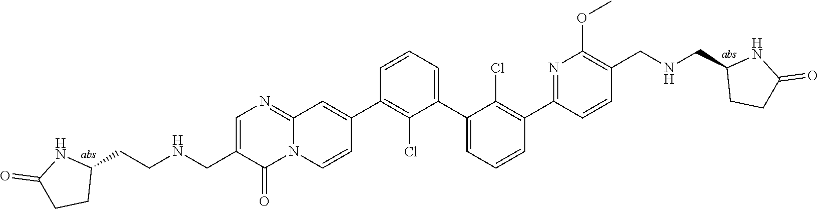 [SMILES: COc1nc(-c2cccc(-c3cccc(-c4ccn5c(=O)c(CNCC[C@@H]6CCC(=O)N6)cnc5c4)c3Cl)c2Cl)ccc1CNC[C@@H]1CCC(=O)N1]

A solution of tert-butyl N-[[6-[2-chloro-3-[2-chloro-3-[4-oxo-3-[[2-[(2S)-5-oxopyrrolidin-2-yl]ethylamino]methyl]pyrido[1,2-a]pyrimidin-8-yl]phenyl]phenyl]-2-methoxy-3-pyridyl]methyl]-N-[[(2S)-5-oxopyrrolidin-2-yl]methyl]carbamate (20 mg, 0.02 mmol) in DCM/TFA (2:1) was stirred for 30 minutes, concentrated and purified by reversed phase chromatography to give 8-[2-chloro-3-[2-chloro-3-[6-methoxy-5-[[[(2S)-5-oxopyrrolidin-2-yl]methylamino]methyl]-2-pyridyl]phenyl]phenyl]-3-[[2-[(2S)-5-oxopyrrolidin-2-yl]ethylamino]methyl]pyrido[1,2-a]pyrimidin-4-one (15 mg, 85.1%). MS: m/z found 740.3 [M+H]⁺. ¹H NMR (400 MHz, Methanol-d₄): δ 9.16 (dt, J=7.4, 1.0 Hz, 1H), 8.45 (s, 1H), 7.81 (dt, J=1.9, 0.9 Hz, 1H), 7.77-7.72 (m, 1H), 7.69-7.56 (m, 4H), 7.54-7.49 (m, 2H), 7.40 (dt, J=7.6, 1.4 Hz, 1H), 7.25 (dd, J=7.4, 1.0 Hz, 1H), 4.02 (s, 3H), 4.01 (s, 2H), 3.89-3.80 (m, 3H), 3.75 (t, J=6.6 Hz, 1H), 2.88 (dq, J=6.5, 3.2, 2.8 Hz, 2H), 2.78-2.64 (m, 2H), 2.38-2.25 (m, 6H), 1.92-1.72 (m, 4H).

### Example 215: 8-[2-chloro-3-[2-chloro-3-[6-methoxy-5-[[[(2S)-5-oxopyrrolidin-2-yl]methylamino]methyl]-2-pyridyl]phenyl]phenyl]-3-[[2-(1H-imidazol-4-yl)ethylamino]methyl]pyrido[1,2-a]pyrimidin-4-one

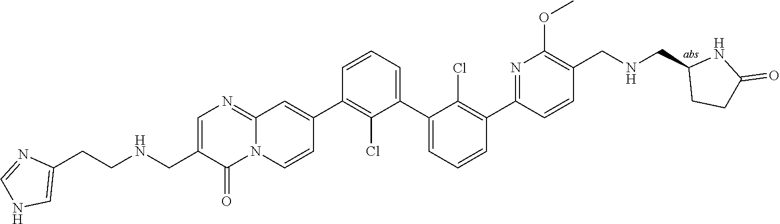 [SMILES: COc1nc(-c2cccc(-c3cccc(-c4ccn5c(=O)c(CNCCc6c[nH]cn6)cnc5c4)c3Cl)c2Cl)ccc1CNC[C@@H]1CCC(=O)N1]

### tert-Butyl N-[[6-[2-chloro-3-[2-chloro-3-[3-[[2-(1H-imidazol-4-yl)ethylamino]methyl]-4-oxo-pyrido[1,2-a]pyrimidin-8-yl]phenyl]phenyl]-2-methoxy-3-pyridyl]methyl]-N-[[(2S)-5-oxopyrrolidin-2-yl]methyl]carbamate

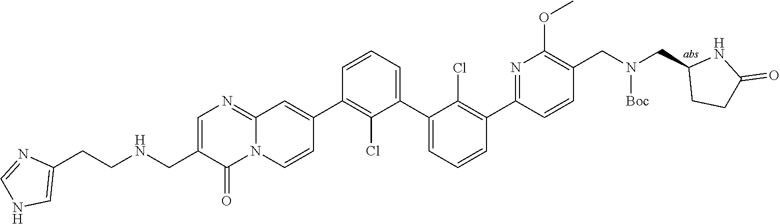 [SMILES: COc1nc(-c2cccc(-c3cccc(-c4ccn5c(=O)c(CNCCc6c[nH]cn6)cnc5c4)c3Cl)c2Cl)ccc1CN(C[C@@H]1CCC(=O)N1)C(=O)OC(C)(C)C]

A mixture of tert-butyl N-[[6-[2-chloro-3-[2-chloro-3-(3-formyl-4-oxo-pyrido[1,2-a]pyrimidin-8-yl)phenyl]phenyl]-2-methoxy-3-pyridyl]methyl]-N-[[(2S)-5-oxopyrrolidin-2-yl]methyl]carbamate (30 mg, 0.04 mmol), 2-(1H-imidazol-4-yl)ethanamine (9 mg, 0.08 mmol) and acetic acid (2 uL, 2.5 mg, 0.04 mmol) in MeOH/THF (2:1) was stirred for 2 hours at rt. NaBH₃CN (5.17 mg, 0.08 mmol) was added, the mixture was stirred for 2 hours, quenched with water, and directly purified by reversed phase chromatography to give tert-butyl N-[[6-[2-chloro-3-[2-chloro-3-[3-[[2-(1H-imidazol-4-yl)ethylamino]methyl]-4-oxo-pyrido[1,2-a]pyrimidin-8-yl]phenyl]phenyl]-2-methoxy-3-pyridyl]methyl]-N-[[(2S)-5-oxopyrrolidin-2-yl]methyl]carbamate (29 mg, 85.5%). MS: m/z found 823.3 [M+H]⁺.

### 8-[2-chloro-3-[2-chloro-3-[6-methoxy-5-[[[(2S)-5-oxopyrrolidin-2-yl]methylamino]methyl]-2-pyridyl]phenyl]phenyl]-3-[[2-(1H-imidazol-4-yl)ethylamino]methyl]pyrido[1,2-a]pyrimidin-4-one

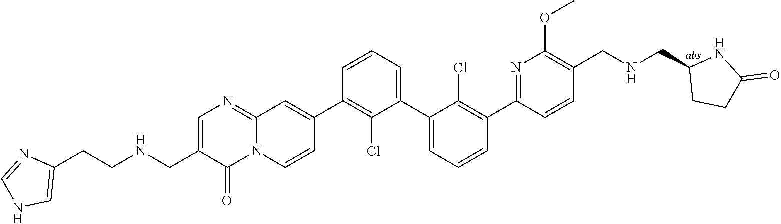 [SMILES: COc1nc(-c2cccc(-c3cccc(-c4ccn5c(=O)c(CNCCc6c[nH]cn6)cnc5c4)c3Cl)c2Cl)ccc1CNC[C@@H]1CCC(=O)N1]

A solution of tert-butyl N-[[6-[2-chloro-3-[2-chloro-3-[3-[[2-(1H-imidazol-4-yl)ethylamino]methyl]-4-oxo-pyrido[1,2-a]pyrimidin-8-yl]phenyl]phenyl]-2-methoxy-3-pyridyl]methyl]-N-[[(2S)-5-oxopyrrolidin-2-yl]methyl]carbamate (23 mg, 0.03 mmol) in DCM/TFA (2:1) was stirred for 30 minutes, concentrated and purified by reversed phase chromatography to give 8-[2-chloro-3-[2-chloro-3-[6-methoxy-5-[[[(2S)-5-oxopyrrolidin-2-yl]methylamino]methyl]-2-pyridyl]phenyl]phenyl]-3-[[2-(1H-imidazol-4-yl)ethylamino]methyl]pyrido[1,2-a]pyrimidin-4-one (18 mg, 89.1%). MS: m/z found 723.2 [M+H]⁺. ¹H NMR (400 MHz, Methanol-d₄): δ 9.22-9.13 (m, 1H), 8.49 (d, J=2.0 Hz, 1H), 7.85 (dt, J=2.1, 1.1 Hz, 1H), 7.76 (dd, J=7.4, 1.9 Hz, 1H), 7.70-7.59 (m, 5H), 7.56-7.48 (m, 2H), 7.41 (dt, J=7.6, 1.9 Hz, 1H), 7.26 (dd, J=7.4, 2.0 Hz, 1H), 7.00 (d, J=1.3 Hz, 1H), 4.24 (s, 2H), 4.03 (s, 3H), 3.93-3.86 (m, 2H), 3.86-3.82 (m, 1H), 3.29-3.26 (m, 2H), 2.99 (t, J=7.2 Hz, 2H), 2.82-2.69 (m, 2H), 2.38-2.24 (m, 3H), 1.83 (ddt, J=12.3, 8.4, 5.6 Hz, 1H).

### Example 216: 8-[2-Chloro-3-[2-chloro-3-[6-methoxy-5-[[[(2S)-5-oxopyrrolidin-2-yl]methylamino]methyl]-2-pyridyl]phenyl]phenyl]-3-[[2-(1,2,4-triazol-4-yl)ethylamino]methyl]pyrido[1,2-a]pyrimidin-4-one

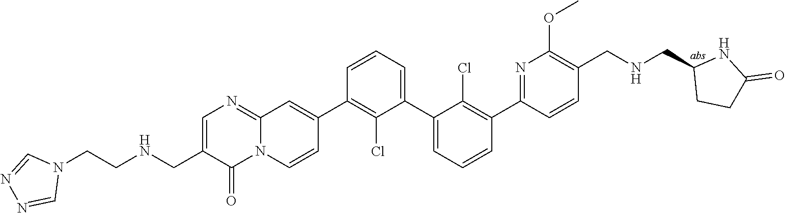 [SMILES: COc1nc(-c2cccc(-c3cccc(-c4ccn5c(=O)c(CNCCn6cnnc6)cnc5c4)c3Cl)c2Cl)ccc1CNC[C@@H]1CCC(=O)N1]

### tert-Butyl N-[[6-[2-chloro-3-[2-chloro-3-[4-oxo-3-[[2-(1,2,4-triazol-4-yl)ethylamino]methyl]pyrido[1,2-a]pyrimidin-8-yl]phenyl]phenyl]-2-methoxy-3-pyridyl]methyl]-N-[[(2S)-5-oxopyrrolidin-2-yl]methyl]carbamate

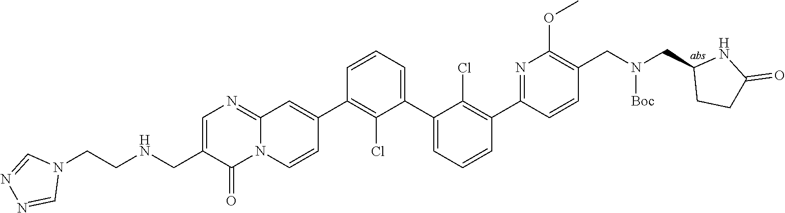 [SMILES: COc1nc(-c2cccc(-c3cccc(-c4ccn5c(=O)c(CNCCn6cnnc6)cnc5c4)c3Cl)c2Cl)ccc1CN(C[C@@H]1CCC(=O)N1)C(=O)OC(C)(C)C]

A mixture of tert-butyl N-[[6-[2-chloro-3-[2-chloro-3-(3-formyl-4-oxo-pyrido[1,2-a]pyrimidin-8-yl)phenyl]phenyl]-2-methoxy-3-pyridyl]methyl]-N-[[(2S)-5-oxopyrrolidin-2-yl]methyl]carbamate (30 mg, 0.04 mmol), 2-(1,2,4-triazol-4-yl)ethanamine (9 mg, 0.08 mmol) and acetic acid (2 uL, 2.5 mg, 0.04 mmol) in MeOH/THF (2:1) was stirred for 2 hours at rt. NaBH₃CN (5 mg, 0.08 mmol) was added, the mixture was stirred for 2 hours, quenched with water, and directly purified by reversed phase chromatography to give tert-butyl N-[[6-[2-chloro-3-[2-chloro-3-[4-oxo-3-[[2-(1,2,4-triazol-4-yl)ethylamino]methyl]pyrido[1,2-a]pyrimidin-8-yl]phenyl]phenyl]-2-methoxy-3-pyridyl]methyl]-N-[[(2S)-5-oxopyrrolidin-2-yl]methyl]carbamate (28 mg, 82.5%). MS: m/z found 824.3 [M+H]⁺.

### 8-[2-Chloro-3-[2-chloro-3-[6-methoxy-5-[[[(2S)-5-oxopyrrolidin-2-yl]methylamino]methyl]-2-pyridyl]phenyl]phenyl]-3-[[2-(1,2,4-triazol-4-yl)ethylamino]methyl]pyrido[1,2-a]pyrimidin-4-one

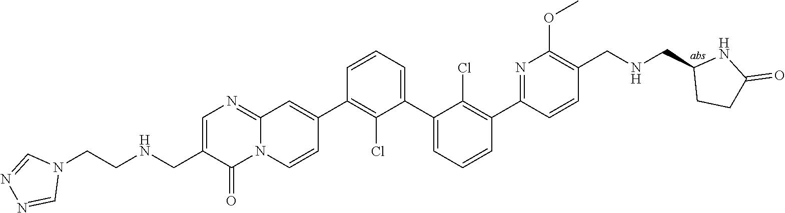 [SMILES: COc1nc(-c2cccc(-c3cccc(-c4ccn5c(=O)c(CNCCn6cnnc6)cnc5c4)c3Cl)c2Cl)ccc1CNC[C@@H]1CCC(=O)N1]

A solution of tert-butyl N-[[6-[2-chloro-3-[2-chloro-3-[4-oxo-3-[[2-(1,2,4-triazol-4-yl)ethylamino]methyl]pyrido[1,2-a]pyrimidin-8-yl]phenyl]phenyl]-2-methoxy-3-pyridyl]methyl]-N-[[(2S)-5-oxopyrrolidin-2-yl]methyl]carbamate (25 mg, 0.03 mmol) in DCM/TFA (2:1) was stirred for 30 minutes, concentrated and purified by reversed phase chromatography to give 8-[2-chloro-3-[2-chloro-3-[6-methoxy-5-[[[(2S)-5-oxopyrrolidin-2-yl]methylamino]methyl]-2-pyridyl]phenyl]phenyl]-3-[[2-(1,2,4-triazol-4-yl)ethylamino]methyl]pyrido[1,2-a]pyrimidin-4-one (20 mg, 91.1%). MS: m/z found 724.2 [M+H]⁺. ¹H NMR (400 MHz, Methanol-d₄): δ 9.12 (dt, J=7.4, 1.0 Hz, 1H), 8.65-8.57 (m, 2H), 8.36 (s, 1H), 7.77-7.73 (m, 2H), 7.66 (dt, J=7.7, 1.6 Hz, 1H), 7.65-7.57 (m, 2H), 7.55-7.48 (m, 3H), 7.40 (dt, J=7.6, 1.5 Hz, 1H), 7.26 (dd, J=7.4, 1.3 Hz, 1H), 4.28 (t, J=6.0 Hz, 2H), 4.03 (s, 3H), 3.93-3.82 (m, 5H), 3.08-3.00 (m, 2H), 2.82-2.69 (m, 2H), 2.39-2.23 (m, 3H), 1.88-1.77 (m, 1H).

### Example 219: 8-[2-Chloro-3-[2-chloro-3-[6-methoxy-5-[[[(2S)-5-oxopyrrolidin-2-yl]methylamino]methyl]-2-pyridyl]phenyl]phenyl]-3-[[2-(2-oxopyrrolidin-1-yl)ethylamino]methyl]pyrido[1,2-a]pyrimidin-4-one

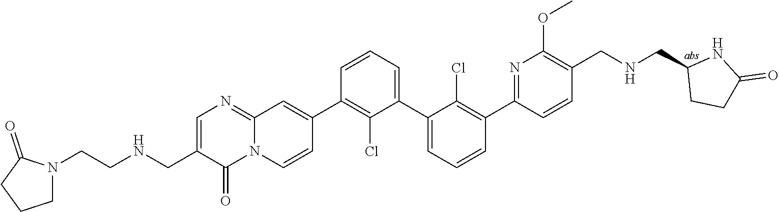 [SMILES: COc1nc(-c2cccc(-c3cccc(-c4ccn5c(=O)c(CNCCN6CCCC6=O)cnc5c4)c3Cl)c2Cl)ccc1CNC[C@@H]1CCC(=O)N1]

### tert-Butyl N-[[6-[2-chloro-3-[2-chloro-3-[4-oxo-3-[[2-(2-oxopyrrolidin-1-yl)ethylamino]methyl]pyrido[1,2-a]pyrimidin-8-yl]phenyl]phenyl]-2-methoxy-3-pyridyl]methyl]-N-[[(2S)-5-oxopyrrolidin-2-yl]methyl]carbamate

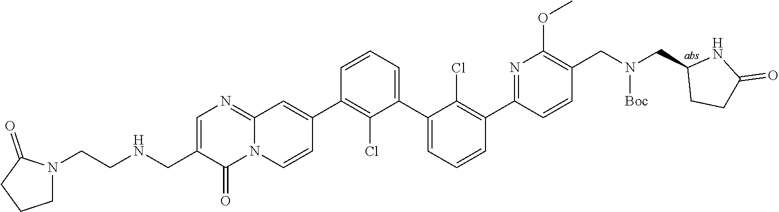 [SMILES: COc1nc(-c2cccc(-c3cccc(-c4ccn5c(=O)c(CNCCN6CCCC6=O)cnc5c4)c3Cl)c2Cl)ccc1CN(C[C@@H]1CCC(=O)N1)C(=O)OC(C)(C)C]

A mixture of tert-butyl N-[[6-[2-chloro-3-[2-chloro-3-(3-formyl-4-oxo-pyrido[1,2-a]pyrimidin-8-yl)phenyl]phenyl]-2-methoxy-3-pyridyl]methyl]-N-[[(2S)-5-oxopyrrolidin-2-yl]methyl]carbamate (30 mg, 0.04 mmol), 1-(2-aminoethyl)pyrrolidin-2-one (11 mg, 0.08 mmol) and acetic acid (2 uL, 2.5 mg, 0.04 mmol) in MeOH/THF (2:1) was stirred for 2 hours at rt. NaBH₃CN (5 mg, 0.08 mmol) was added, the mixture was stirred for 2 hours, quenched with water, and directly purified by reversed phase chromatography to give tert-butyl N-[[6-[2-chloro-3-[2-chloro-3-[4-oxo-3-[[2-(2-oxopyrrolidin-1-yl)ethylamino]methyl]pyrido[1,2-a]pyrimidin-8-yl]phenyl]phenyl]-2-methoxy-3-pyridyl]methyl]-N-[[(2S)-5-oxopyrrolidin-2-yl]methyl]carbamate (30 mg, 86.7%). MS: m/z found 840.3 [M+H]⁺.

### 8-[2-Chloro-3-[2-chloro-3-[6-methoxy-5-[[[(2S)-5-oxopyrrolidin-2-yl]methylamino]methyl]-2-pyridyl]phenyl]phenyl]-3-[[2-(2-oxopyrrolidin-1-yl)ethylamino]methyl]pyrido[1,2-a]pyrimidin-4-one

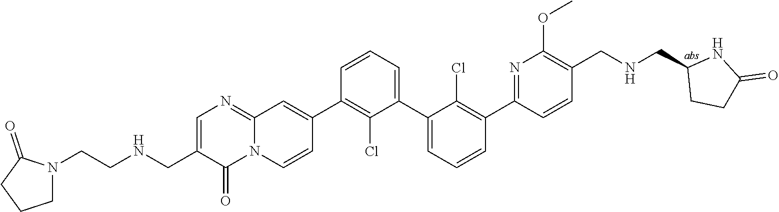 [SMILES: COc1nc(-c2cccc(-c3cccc(-c4ccn5c(=O)c(CNCCN6CCCC6=O)cnc5c4)c3Cl)c2Cl)ccc1CNC[C@@H]1CCC(=O)N1]

A solution of tert-butyl-N-[[6-[2-chloro-3-[2-chloro-3-[4-oxo-3-[[2-(2-oxopyrrolidin-1-yl)ethylamino]methyl]pyrido[1,2-a]pyrimidin-8-yl]phenyl]phenyl]-2-methoxy-3-pyridyl]methyl]-N-[[(2S)-5-oxopyrrolidin-2-yl]methyl]carbamate (20 mg, 0.02 mmol) in DCM/TFA (2:1) was stirred for 30 minutes, concentrated and purified by reversed phase chromatography to give 8-[2-chloro-3-[2-chloro-3-[6-methoxy-5-[[[(2S)-5-oxopyrrolidin-2-yl]methylamino]methyl]-2-pyridyl]phenyl]phenyl]-3-[[2-(2-oxopyrrolidin-1-yl)ethylamino]methyl]pyrido[1,2-a]pyrimidin-4-one (16 mg, 90.8%). MS: m/z found 740.3 [M+H]⁺. ¹H NMR (400 MHz, Methanol-d₄): δ 9.17 (dd, J=7.4, 0.6 Hz, 1H), 8.50 (s, 1H), 8.45 (s, 2H), 7.84 (dd, J=1.9, 0.6 Hz, 1H), 7.81 (d, J=7.5 Hz, 1H), 7.67 (dd, J=7.7, 1.7 Hz, 1H), 7.66-7.59 (m, 3H), 7.55-7.50 (m, 2H), 7.41 (dd, J=7.6, 1.7 Hz, 1H), 7.29 (d, J=7.5 Hz, 1H), 4.25 (s, 2H), 4.07-4.00 (m, 5H), 3.94 (dt, J=10.0, 3.7 Hz, 1H), 3.63 (t, J=6.0 Hz, 2H), 3.52 (t, J=7.1 Hz, 2H), 3.23 (dd, J=7.0, 4.9 Hz, 2H), 3.00-2.86 (m, 2H), 2.42 (t, J=8.1 Hz, 2H), 2.39-2.29 (m, 3H), 2.10 (tt, J=7.9, 6.8 Hz, 2H), 1.86 (dtd, J=11.2, 9.3, 8.7, 5.4 Hz, 1H).

### Example 220: Methyl (3S)-4-[[8-[2-chloro-3-[2-chloro-3-[6-methoxy-5-[[[(2S)-5-oxopyrrolidin-2-yl]methylamino]methyl]-2-pyridyl]phenyl]phenyl]-4-oxo-pyrido[1,2-a]pyrimidin-3-yl]methylamino]-3-hydroxy-butanoate

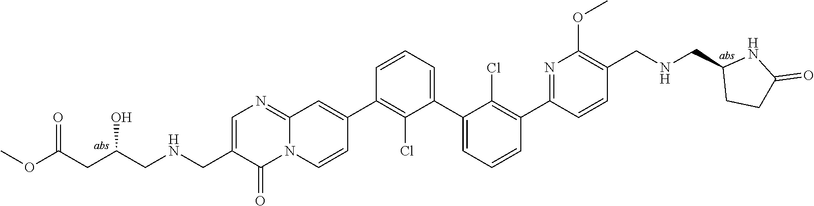 [SMILES: COC(=O)C[C@H](O)CNCc1cnc2cc(-c3cccc(-c4cccc(-c5ccc(CNC[C@@H]6CCC(=O)N6)c(OC)n5)c4Cl)c3Cl)ccn2c1=O]

### Methyl (3S)-4-[[8-[3-[3-[5-[[tert-butoxycarbonyl-[[(2S)-5-oxopyrrolidin-2-yl]methyl]amino]methyl]-6-methoxy-2-pyridyl]-2-chloro-phenyl]-2-chloro-phenyl]-4-oxo-pyrido[1,2-a]pyrimidin-3-yl]methylamino]-3-hydroxy-butanoate

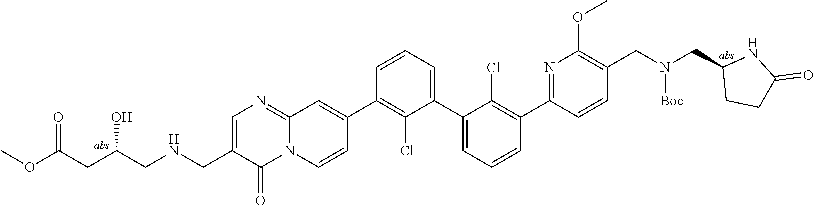 [SMILES: COC(=O)C[C@H](O)CNCc1cnc2cc(-c3cccc(-c4cccc(-c5ccc(CN(C[C@@H]6CCC(=O)N6)C(=O)OC(C)(C)C)c(OC)n5)c4Cl)c3Cl)ccn2c1=O]

A mixture of tert-butyl N-[[6-[2-chloro-3-[2-chloro-3-(3-formyl-4-oxo-pyrido[1,2-a]pyrimidin-8-yl)phenyl]phenyl]-2-methoxy-3-pyridyl]methyl]-N-[[(2S)-5-oxopyrrolidin-2-yl]methyl]carbamate (40 mg, 0.05 mmol), methyl (3S)-4-amino-3-hydroxy-butanoate (15 mg, 0.11 mmol), and acetic acid (3 uL, 3.3 mg, 0.05 mmol) in MeOH/THF (2:1) was stirred for 2 hours at rt. NaBH₃CN, (7 mg, 0.11 mmol) was added, the mixture was stirred for 2 hours, quenched with water, and directly purified by reversed phase chromatography to give methyl (3S)-4-[[8-[3-[3-[5-[[tert-butoxycarbonyl-[[(2S)-5-oxopyrrolidin-2-yl]methyl]amino]methyl]-6-methoxy-2-pyridyl]-2-chloro-phenyl]-2-chloro-phenyl]-4-oxo-pyrido[1,2-a]pyrimidin-3-yl]methylamino]-3-hydroxy-butanoate (20 mg, 43.1%). MS: m/z found 845.3 [M+H]⁺.

### Methyl (3S)-4-[[8-[2-chloro-3-[2-chloro-3-[6-methoxy-5-[[[(2S)-5-oxopyrrolidin-2-yl]methylamino]methyl]-2-pyridyl]phenyl]phenyl]-4-oxo-pyrido[1,2-a]pyrimidin-3-yl]methylamino]-3-hydroxy-butanoate

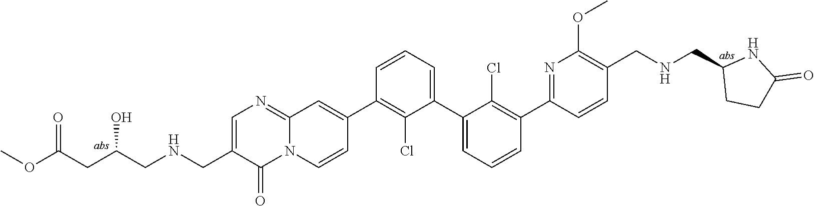 [SMILES: COC(=O)C[C@H](O)CNCc1cnc2cc(-c3cccc(-c4cccc(-c5ccc(CNC[C@@H]6CCC(=O)N6)c(OC)n5)c4Cl)c3Cl)ccn2c1=O]

A solution of methyl (3 S)-4-[[8-[3-[3-[5-[[tert-butoxycarbonyl-[[(2S)-5-oxopyrrolidin-2-yl]methyl]amino]methyl]-6-methoxy-2-pyridyl]-2-chloro-phenyl]-2-chloro-phenyl]-4-oxo-pyrido[1,2-a]pyrimidin-3-yl]methylamino]-3-hydroxy-butanoate (20 mg, 0.02 mmol) in DCM/TFA (2:1) was stirred for 30 minutes, concentrated and purified by reversed phase chromatography to give methyl (3S)-4-[[8-[2-chloro-3-[2-chloro-3-[6-methoxy-5-[[[(2S)-5-oxopyrrolidin-2-yl]methylamino]methyl]-2-pyridyl]phenyl]phenyl]-4-oxo-pyrido[1,2-a]pyrimidin-3-yl]methylamino]-3-hydroxy-butanoate (17 mg, 96.4%). MS: m/z found 745.2 [M+H]⁺. ¹H NMR (400 MHz, Methanol-d₄): δ 9.18 (dt, J=7.3, 0.8 Hz, 1H), 8.52 (d, J=0.7 Hz, 1H), 8.46 (s, 2H), 7.85 (dd, J=1.9, 0.8 Hz, 1H), 7.81 (dd, J=7.5, 0.7 Hz, 1H), 7.67 (ddd, J=7.7, 1.7, 0.7 Hz, 1H), 7.65-7.59 (m, 3H), 7.55-7.50 (m, 2H), 7.41 (ddd, J=7.6, 1.8, 0.8 Hz, 1H), 7.29 (dd, J=7.5, 0.8 Hz, 1H), 4.35 (dtt, J=8.5, 5.5, 3.0 Hz, 1H), 4.30 (s, 2H), 4.10-4.00 (m, 5H), 3.98-3.90 (m, 1H), 3.69 (s, 3H), 3.25 (ddd, J=12.6, 3.1, 0.7 Hz, 1H), 3.06 (dd, J=12.7, 9.8 Hz, 1H), 3.00-2.88 (m, 2H), 2.64-2.52 (m, 2H), 2.41-2.30 (m, 3H), 1.91-1.81 (m, 1H).

### Example 231: Methyl (3S)-4-[[6-[2-chloro-3-[2-chloro-3-[3-[[[(2S)-2-hydroxy-4-methoxy-4-oxo-butyl]amino]methyl]-4-oxo-pyrido[1,2-a]pyrimidin-8-yl]phenyl]phenyl]-2-methoxy-3-pyridyl]methylamino]-3-hydroxy-butanoate

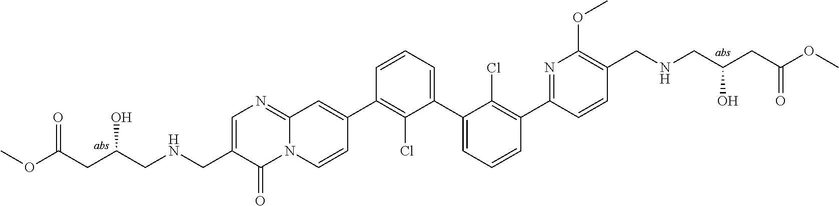

A mixture of 8-[2-chloro-3-[2-chloro-3-(5-formyl-6-methoxy-2-pyridyl)phenyl]phenyl]-4-oxo-pyrido[1,2-a]pyrimidine-3-carbaldehyde (30.00 mg, 0.06 mmol), methyl (3S)-4-amino-3-hydroxy-butanoate (30 mg, 0.23 mmol) and acetic acid (6.5 uL, 6.8 mg, 0.11 mmol) in MeOH/THF (1:1) was stirred for 1.5 hour at rt. NaBH₃CN (14 mg, 0.23 mmol) was added, the mixture was stirred for additional 1 hour, and then quenched with water and purified by reversed phase chromatography to give methyl (3S)-4-[[6-[2-chloro-3-[2-chloro-3-[3-[[[(2S)-2-hydroxy-4-methoxy-4-oxo-butyl]amino]methyl]-4-oxo-pyrido[1,2-a]pyrimidin-8-yl]phenyl]phenyl]-2-methoxy-3-pyridyl]methylamino]-3-hydroxy-butanoate (25 mg, 57.8%). MS: m/z found 764.2 [M+H]⁺. ¹H NMR (400 MHz, Methanol-d₄): δ 9.17 (dd, J=7.4, 0.7 Hz, 1H), 8.52 (br s, NH), 8.48 (s, 1H), 7.86-7.78 (m, 2H), 7.68 (dd, J=7.7, 1.8 Hz, 1H), 7.66-7.57 (m, 3H), 7.56-7.50 (m, 2H), 7.42 (dd, J=7.6, 1.7 Hz, 1H), 7.32 (d, J=7.5 Hz, 1H), 4.29 (dq, J=8.7, 4.3, 3.2 Hz, 2H), 4.22-4.09 (m, 4H), 4.06 (s, 3H), 3.69 (s, 3H), 3.69 (s, 3H), 3.07 (ddd, J=17.6, 12.5, 3.3 Hz, 2H), 2.92 (td, J=12.5, 9.3 Hz, 2H), 2.64-2.48 (m, 4H).

### Example 238: (3S)-4-[[8-[2-chloro-3-[2-chloro-3-[6-methoxy-5-[[[(2S)-5-oxopyrrolidin-2-yl]methylamino]methyl]-2-pyridyl]phenyl]phenyl]-4-oxo-pyrido[1,2-a]pyrimidin-3-yl]methylamino]-3-hydroxy-butanamide

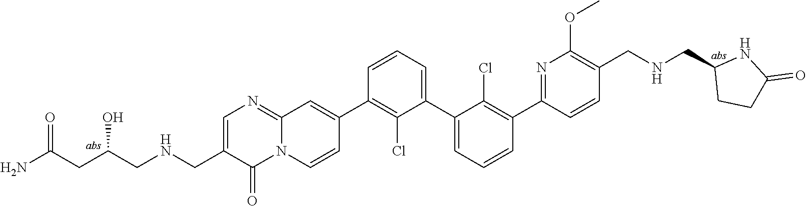 [SMILES: COc1nc(-c2cccc(-c3cccc(-c4ccn5c(=O)c(CNC[C@@H](O)CC(N)=O)cnc5c4)c3Cl)c2Cl)ccc1CNC[C@@H]1CCC(=O)N1]

### tert-Butyl N-[[6-[3-[3-[3-[[[(2S)-4-amino-2-hydroxy-4-oxo-butyl]amino]methyl]-4-oxo-pyrido[1,2-a]pyrimidin-8-yl]-2-chloro-phenyl]-2-chloro-phenyl]-2-methoxy-3-pyridyl]methyl]-N-[[(2S)-5-oxopyrrolidin-2-yl]methyl]carbamate

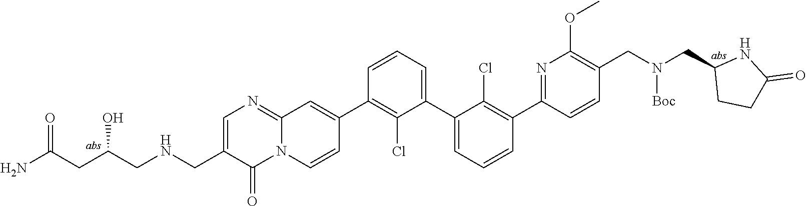 [SMILES: COc1nc(-c2cccc(-c3cccc(-c4ccn5c(=O)c(CNC[C@@H](O)CC(N)=O)cnc5c4)c3Cl)c2Cl)ccc1CN(C[C@@H]1CCC(=O)N1)C(=O)OC(C)(C)C]

A mixture of tert-butyl N-[[6-[2-chloro-3-[2-chloro-3-(3-formyl-4-oxo-pyrido[1,2-a]pyrimidin-8-yl)phenyl]phenyl]-2-methoxy-3-pyridyl]methyl]-N-[[(2S)-5-oxopyrrolidin-2-yl]methyl]carbamate (40 mg, 0.05 mmol), (3S)-4-amino-3-hydroxy-butanamide (13 mg, 0.11 mmol) and acetic acid (3 uL, 3 mg, 0.05 mmol) in MeOH/THF (2:1) was stirred for 2 hours at rt. NaBH₃CN (6.90 mg, 0.11 mmol) was added, the mixture was stirred for 4 hours, quenched with water, and directly purified by reversed phase chromatography to give tert-butyl N-[[6-[3-[3-[3-[[[(2S)-4-amino-2-hydroxy-4-oxo-butyl]amino]methyl]-4-oxo-pyrido[1,2-a]pyrimidin-8-yl]-2-chloro-phenyl]-2-chloro-phenyl]-2-methoxy-3-pyridyl]methyl]-N-[[(2S)-5-oxopyrrolidin-2-yl]methyl]carbamate (20 mg, 43.9%). MS: m/z found 830.3 [M+H]⁺.

### (3S)-4-[[8-[2-Chloro-3-[2-chloro-3-[6-methoxy-5-[[[(2S)-5-oxopyrrolidin-2-yl]methylamino]methyl]-2-pyridyl]phenyl]phenyl]-4-oxo-pyrido[1,2-a]pyrimidin-3-yl]methylamino]-3-hydroxy-butanamide

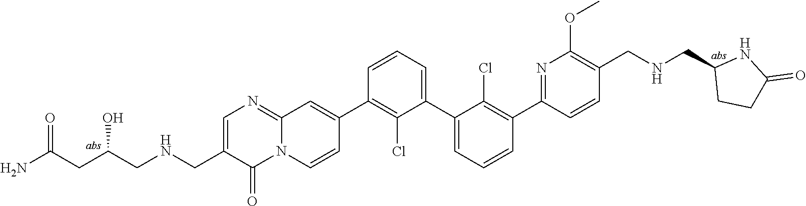 [SMILES: COc1nc(-c2cccc(-c3cccc(-c4ccn5c(=O)c(CNC[C@@H](O)CC(N)=O)cnc5c4)c3Cl)c2Cl)ccc1CNC[C@@H]1CCC(=O)N1]

A solution of tert-butyl N-[[6-[3-[3-[3-[[[(2S)-4-amino-2-hydroxy-4-oxo-butyl]amino]methyl]-4-oxo-pyrido[1,2-a]pyrimidin-8-yl]-2-chloro-phenyl]-2-chloro-phenyl]-2-methoxy-3-pyridyl]methyl]-N-[[(2S)-5-oxopyrrolidin-2-yl]methyl]carbamate (20 mg, 0.02 mmol) in DCM/TFA (2:1) was stirred for 30 minutes, concentrated and purified by reversed phase chromatography to give (3S)-4-[[8-[2-chloro-3-[2-chloro-3-[6-methoxy-5-[[[(2S)-5-oxopyrrolidin-2-yl]methylamino]methyl]-2-pyridyl]phenyl]phenyl]-4-oxo-pyrido[1,2-a]pyrimidin-3-yl]methylamino]-3-hydroxy-butanamide (16 mg, 91.0%). MS: m/z found 730.2 [M+H]⁺. ¹H NMR (400 MHz, Methanol-d₄): δ 9.18 (dd, J=7.3, 2.5 Hz, 1H), 8.53-8.49 (m, 1H), 7.86-7.83 (m, 1H), 7.76 (dd, J=7.6, 2.4 Hz, 1H), 7.69-7.59 (m, 4H), 7.52 (ddt, J=7.6, 6.6, 1.7 Hz, 2H), 7.41 (dq, J=7.6, 1.6 Hz, 1H), 7.29-7.22 (m, 1H), 4.32-4.26 (m, 1H), 4.25 (s, 2H), 4.05-4.00 (m, 3H), 3.94-3.89 (m, 2H), 3.89-3.84 (m, 1H), 3.19 (dt, J=12.7, 2.9 Hz, 1H), 3.05-2.95 (m, 1H), 2.84-2.72 (m, 2H), 2.45 (dt, J=6.2, 1.8 Hz, 2H), 2.35 (dt, J=11.3, 2.8 Hz, 2H), 2.32-2.25 (m, 1H), 1.89-1.77 (m, 1H).

### Example 249: 8-[2-Chloro-3-[2-chloro-3-[6-methoxy-5-[[[(2S)-5-oxopyrrolidin-2-yl]methylamino]methyl]-2-pyridyl]phenyl]phenyl]-3-[[(5-oxo-6-azaspiro[3.4]octan-2-yl)amino]methyl]pyrido[1,2-a]pyrimidin-4-one

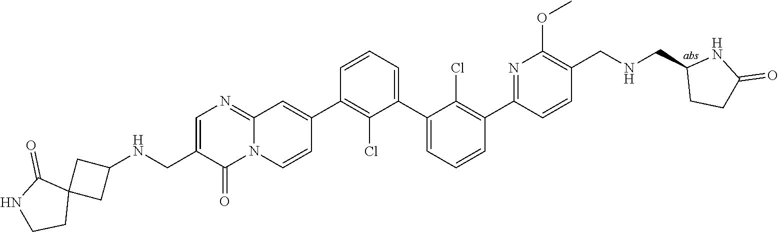 [SMILES: COc1nc(-c2cccc(-c3cccc(-c4ccn5c(=O)c(CNC6CC7(CCNC7=O)C6)cnc5c4)c3Cl)c2Cl)ccc1CNC[C@@H]1CCC(=O)N1]

### tert-Butyl N-[[6-[2-chloro-3-[2-chloro-3-[4-oxo-3-[[(5-oxo-6-azaspiro[3.4]octan-2-yl)amino]methyl]pyrido[1,2-a]pyrimidin-8-yl]phenyl]phenyl]-2-methoxy-3-pyridyl]methyl]-N-[[(2S)-5-oxopyrrolidin-2-yl]methyl]carbamate

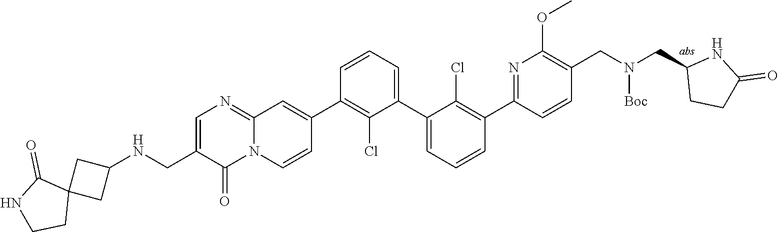 [SMILES: COc1nc(-c2cccc(-c3cccc(-c4ccn5c(=O)c(CNC6CC7(CCNC7=O)C6)cnc5c4)c3Cl)c2Cl)ccc1CN(C[C@@H]1CCC(=O)N1)C(=O)OC(C)(C)C]

A mixture of tert-butyl N-[[6-[2-chloro-3-[2-chloro-3-(3-formyl-4-oxo-pyrido[1,2-a]pyrimidin-8-yl)phenyl]phenyl]-2-methoxy-3-pyridyl]methyl]-N-[[(2S)-5-oxopyrrolidin-2-yl]methyl]carbamate (30 mg, 0.04 mmol), 2-amino-6-azaspiro[3.4]octan-5-one (11.5 mg, 0.08 mmol) and acetic acid (2.5 uL, 2.5 mg, 0.04 mmol) in MeOH/THF (2:1) was stirred for 2 hours at rt. NaBH₃CN (5.17 mg, 0.08 mmol) was added, the mixture was stirred for 2 hours, quenched with water, and directly purified by reversed phase chromatography to give tert-butyl N-[[6-[2-chloro-3-[2-chloro-3-[4-oxo-3-[[(5-oxo-6-azaspiro[3.4]octan-2-yl)amino]methyl]pyrido[1,2-a]pyrimidin-8-yl]phenyl]phenyl]-2-methoxy-3-pyridyl]methyl]-N-[[(2S)-5-oxopyrrolidin-2-yl]methyl]carbamate (30 mg, 85.4%). MS: m/z found 852.3 [M+H]⁺.

### 8-[2-Chloro-3-[2-chloro-3-[6-methoxy-5-[[[(2S)-5-oxopyrrolidin-2-yl]methylamino]methyl]-2-pyridyl]phenyl]phenyl]-3-[[(5-oxo-6-azaspiro[3.4]octan-2-yl)amino]methyl]pyrido[1,2-a]pyrimidin-4-one (racemate)

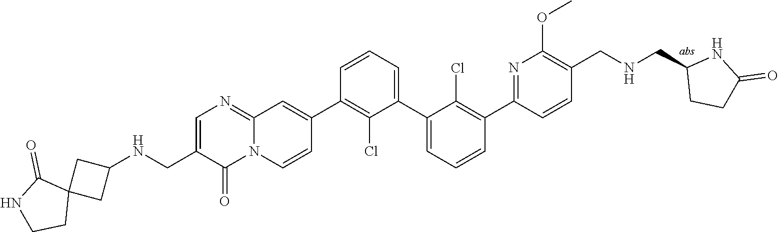 [SMILES: COc1nc(-c2cccc(-c3cccc(-c4ccn5c(=O)c(CNC6CC7(CCNC7=O)C6)cnc5c4)c3Cl)c2Cl)ccc1CNC[C@@H]1CCC(=O)N1]

A solution of tert-butyl N-[[6-[2-chloro-3-[2-chloro-3-[4-oxo-3-[[(5-oxo-6-azaspiro[3.4]octan-2-yl)amino]methyl]pyrido[1,2-a]pyrimidin-8-yl]phenyl]phenyl]-2-methoxy-3-pyridyl]methyl]-N-[[(2S)-5-oxopyrrolidin-2-yl]methyl]carbamate (25 mg, 0.03 mmol) in DCM/TFA (2:1) was stirred for 30 minutes, concentrated and purified by reversed phase chromatography to give 8-[2-chloro-3-[2-chloro-3-[6-methoxy-5-[[[(2S)-5-oxopyrrolidin-2-yl]methylamino]methyl]-2-pyridyl]phenyl]phenyl]-3-[[(5-oxo-6-azaspiro[3.4]octan-2-yl)amino]methyl]pyrido[1,2-a]pyrimidin-4-one (21 mg, 95.2%) as a mixture of two isomers (˜1:2 ratio of isomer 1 to isomer 2). MS: m/z found 752.3 [M+H]⁺.

### Example 250: 8-[2-Chloro-3-[2-chloro-3-[6-methoxy-5-[[[(2S)-5-oxopyrrolidin-2-yl]methylamino]methyl]-2-pyridyl]phenyl]phenyl]-3-[[(5-oxo-6-azaspiro[3.4]octan-2-yl)amino]methyl]pyrido[1,2-a]pyrimidin-4-one (Isomer 1)

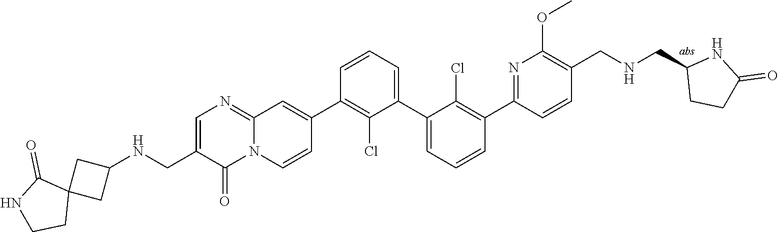 [SMILES: COc1nc(-c2cccc(-c3cccc(-c4ccn5c(=O)c(CNC6CC7(CCNC7=O)C6)cnc5c4)c3Cl)c2Cl)ccc1CNC[C@@H]1CCC(=O)N1]

Racemic mixture was subjected to reversed phase chromatography to give Isomer 1 as a white solid. MS: m/z found 752.3 [M+H]⁺. ¹H NMR (400 MHz, Methanol-d₄): δ 9.18 (dd, J=7.3, 0.7 Hz, 1H), 8.53-8.43 (m, 3H), 7.84 (dd, J=1.9, 0.8 Hz, 1H), 7.77 (d, J=7.5 Hz, 1H), 7.67 (dd, J=7.7, 1.7 Hz, 1H), 7.65-7.59 (m, 3H), 7.55-7.50 (m, 2H), 7.41 (dd, J=7.6, 1.7 Hz, 1H), 7.27 (d, J=7.4 Hz, 1H), 4.10 (s, 2H), 4.04 (s, 3H), 3.98-3.86 (m, 4H), 2.87-2.74 (m, 2H), 2.67 (ddd, J=10.6, 7.9, 3.0 Hz, 2H), 2.39-2.29 (m, 3H), 2.29-2.20 (m, 4H), 1.84 (tt, J=11.8, 6.3 Hz, 1H).

### Example 251: 8-[2-Chloro-3-[2-chloro-3-[6-methoxy-5-[[[(2S)-5-oxopyrrolidin-2-yl]methylamino]methyl]-2-pyridyl]phphenlphenyl]-3-[[(5-oxo-6-azaspiro[3.4]octan-2-yl)amino]methyl]pyrido[1,2-a]pyrimidin-4-one (Isomer 2)

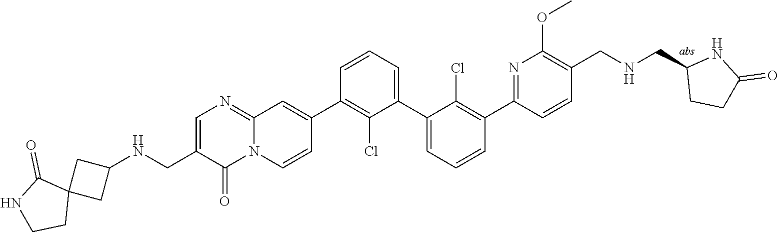 [SMILES: COc1nc(-c2cccc(-c3cccc(-c4ccn5c(=O)c(CNC6CC7(CCNC7=O)C6)cnc5c4)c3Cl)c2Cl)ccc1CNC[C@@H]1CCC(=O)N1]

Racemic mixture was subjected to reversed phase chromatography to give Isomer 2 as a white solid. MS: m/z found 752.3 [M+H]⁺. ¹H NMR (400 MHz, Methanol-d₄): δ 9.18 (dd, J=7.4, 0.8 Hz, 1H), 8.55-8.45 (m, 3H), 7.83 (dd, J=1.9, 0.8 Hz, 1H), 7.76 (d, J=7.5 Hz, 1H), 7.67 (dd, J=7.7, 1.7 Hz, 1H), 7.65-7.59 (m, 3H), 7.54-7.49 (m, 2H), 7.41 (dd, J=7.6, 1.7 Hz, 1H), 7.26 (d, J=7.4 Hz, 1H), 4.09 (s, 2H), 4.03 (s, 3H), 3.93-3.84 (m, 3H), 3.83-3.74 (m, 1H), 3.35-3.32 (m, 2H), 2.81-2.70 (m, 2H), 2.46-2.40 (m, 4H), 2.38-2.28 (m, 5H), 1.88-1.77 (m, 1H).

### Example 252: 8-[2-Chloro-3-[2-chloro-3-[6-methoxy-5-[[[(2S)-5-oxopyrrolidin-2-yl]methylamino]methyl]-2-pyridyl]phenyl]phenyl]-3-[[(3-oxo-2-azabicyclo[2.2.1]heptan-1-yl)methylamino]methyl]pyrido[1,2-a]pyrimidin-4-one

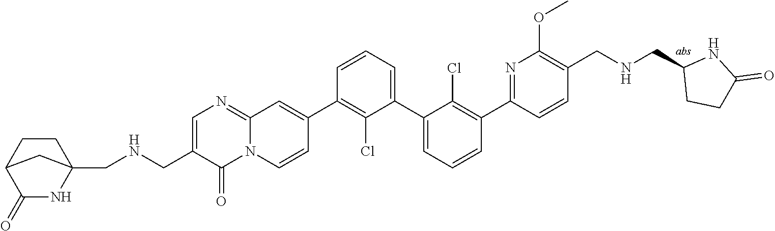 [SMILES: COc1nc(-c2cccc(-c3cccc(-c4ccn5c(=O)c(CNCC67CCC(C6)C(=O)N7)cnc5c4)c3Cl)c2Cl)ccc1CNC[C@@H]1CCC(=O)N1]

### tert-Butyl N-[[6-[2-chloro-3-[2-chloro-3-[4-oxo-3-[[(3-oxo-2-azabicyclo[2.2.1]heptan-1-yl)methylamino]methyl]pyrido[1,2-a]pyrimidin-8-yl]phenyl]phenyl]-2-methoxy-3-pyridyl]methyl]-N-[[(2S)-5-oxopyrrolidin-2-yl]methyl]carbamate

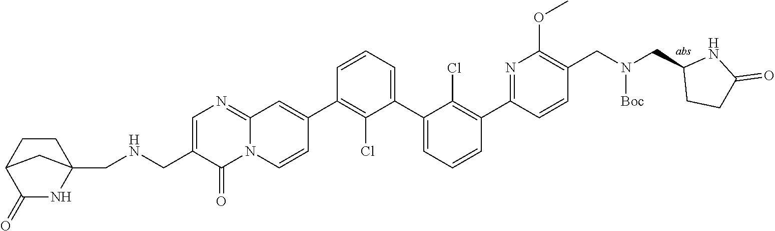 [SMILES: COc1nc(-c2cccc(-c3cccc(-c4ccn5c(=O)c(CNCC67CCC(C6)C(=O)N7)cnc5c4)c3Cl)c2Cl)ccc1CN(C[C@@H]1CCC(=O)N1)C(=O)OC(C)(C)C]

A mixture of tert-butyl N-[[6-[2-chloro-3-[2-chloro-3-(3-formyl-4-oxo-pyrido[1,2-a]pyrimidin-8-yl)phenyl]phenyl]-2-methoxy-3-pyridyl]methyl]-N-[[(2S)-5-oxopyrrolidin-2-yl]methyl]carbamate (30 mg, 0.04 mmol), 1-(aminomethyl)-2-azabicyclo[2.2.1]heptan-3-one (11.5 mg, 0.08 mmol) and acetic acid (2.5 uL, 2.g mg, 0.04 mmol) in MeOH/THF (2:1) was stirred for 2 hours at rt. NaBH₃CN (5 mg, 0.08 mmol) was added, the mixture was stirred for 2 hours, quenched with water, and directly purified by reversed phase chromatography to give tert-butyl N-[[6-[2-chloro-3-[2-chloro-3-[4-oxo-3-[[(3-oxo-2-azabicyclo[2.2.1]heptan-1-yl)methylamino]methyl]pyrido[1,2-a]pyrimidin-8-yl]phenyl]phenyl]-2-methoxy-3-pyridyl]methyl]-N-[[(2S)-5-oxopyrrolidin-2-yl]methyl]carbamate (31 mg, 88.3%). MS: m/z found 852.3 [M+H]⁺.

### 8-[2-Chloro-3-[2-chloro-3-[6-methoxy-5-[[[(2S)-5-oxopyrrolidin-2-yl]methylamino]methyl]-2-pyridyl]phenyl]phenyl]-3-[[(3-oxo-2-azabicyclo[2.2.1]heptan-1-yl)methylamino]methyl]pyrido[1,2-a]pyrimidin-4-one

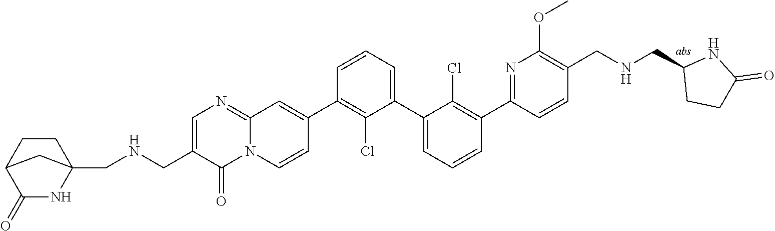 [SMILES: COc1nc(-c2cccc(-c3cccc(-c4ccn5c(=O)c(CNCC67CCC(C6)C(=O)N7)cnc5c4)c3Cl)c2Cl)ccc1CNC[C@@H]1CCC(=O)N1]

A solution of tert-butyl N-[[6-[2-chloro-3-[2-chloro-3-[4-oxo-3-[[(3-oxo-2-azabicyclo[2.2.1]heptan-1-yl)methylamino]methyl]pyrido[1,2-a]pyrimidin-8-yl]phenyl]phenyl]-2-methoxy-3-pyridyl]methyl]-N-[[(2S)-5-oxopyrrolidin-2-yl]methyl]carbamate (25 mg, 0.03 mmol) in DCM/TFA (2:1) was stirred for 30 minutes, concentrated and purified by reversed phase chromatography to give 8-[2-chloro-3-[2-chloro-3-[6-methoxy-5-[[[(2S)-5-oxopyrrolidin-2-yl]methylamino]methyl]-2-pyridyl]phenyl]phenyl]-3-[[(3-oxo-2-azabicyclo[2.2.1]heptan-1-yl)methylamino]methyl]pyrido[1,2-a]pyrimidin-4-one (21 mg, 95.2%). MS: m/z found 752.3 [M+H]⁺. ¹H NMR (400 MHz, Methanol-d₄): δ 9.17 (dt, J=7.5, 0.9 Hz, 1H), 8.47 (s, 1H), 8.44 (s, 1H), 7.84-7.77 (m, 2H), 7.69-7.65 (m, 1H), 7.65-7.57 (m, 3H), 7.53 (dddd, J=7.7, 4.7, 2.7, 1.2 Hz, 2H), 7.42 (ddd, J=7.6, 1.8, 0.9 Hz, 1H), 7.29 (dd, J=7.5, 0.9 Hz, 1H), 4.09 (s, 2H), 4.05 (s, 3H), 4.01 (d, J=3.5 Hz, 2H), 3.96-3.89 (m, 1H), 3.26-3.16 (m, 2H), 2.96-2.84 (m, 2H), 2.72 (d, J=4.0 Hz, 1H), 2.43-2.29 (m, 3H), 2.09-2.00 (m, 1H), 1.88 (tdd, J=12.7, 9.3, 5.1 Hz, 3H), 1.69-1.57 (m, 2H), 1.53 (dd, J=9.3, 1.3 Hz, 1H).

### Example 253: 8-[2-Chloro-3-[2-chloro-3-[6-methoxy-5-[[[(2S)-5-oxopyrrolidin-2-yl]methylamino]methyl]-2-pyridyl]phenyl]phenyl]-3-[[(4,4-difluoro-5-oxo-pyrrolidin-2-yl)methylamino]methyl]pyrido[1,2-a]pyrimidin-4-one

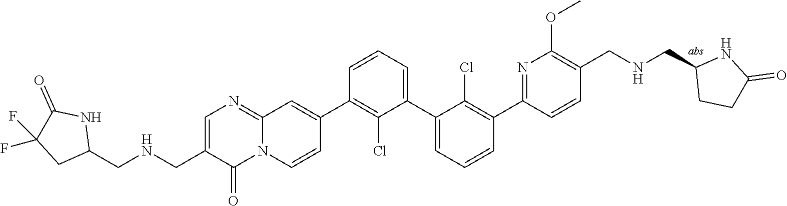 [SMILES: COc1nc(-c2cccc(-c3cccc(-c4ccn5c(=O)c(CNCC6CC(F)(F)C(=O)N6)cnc5c4)c3Cl)c2Cl)ccc1CNC[C@@H]1CCC(=O)N1]

### tert-Butyl N-116-2-chloro-3-[2-chloro-3-[3-h(4,4-difluoro-5-oxo-pyrrolidin-2-yl)methylamino]methyl]p-4-oxo-pyrido[1,2-a pyrimidin-8-yl[phenyl]phylyl]-2-moethoxy-3-pyridyl]methyl]-N-[[(2S)-5-oxopyrrolidin-2-yl]methyl]carbamate

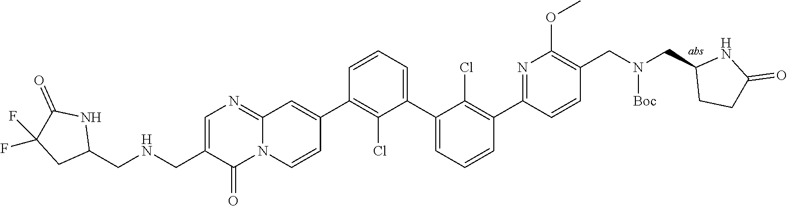 [SMILES: COc1nc(-c2cccc(-c3cccc(-c4ccn5c(=O)c(CNCC6CC(F)(F)C(=O)N6)cnc5c4)c3Cl)c2Cl)ccc1CN(C[C@@H]1CCC(=O)N1)C(=O)OC(C)(C)C]

A mixture of tert-butyl N-[[6-[2-chloro-3-[2-chloro-3-(3-formyl-4-oxo-pyrido[1,2-a]pyrimidin-8-yl)phenyl]phenyl]-2-methoxy-3-pyridyl]methyl]-N-[[(2S)-5-oxopyrrolidin-2-yl]methyl]carbamate (30 mg, 0.04 mmol), 5-(aminomethyl)-3, 3-difluoro-pyrrolidin-2-one (12 mg, 0.08 mmol) and acetic acid (2.5 uL, 2.5 mg, 0.04 mmol) in MOH/TF (2:1) was stirred for 2 hours at rt. NaBH₃CN (5 mg, 0.08 mmol) was added, the mixture was stirred for 2 hours, quenched with water, and directly purified by reversed phase chromatography to give tert-butyl N-[[6-[2-chloro-3-[2-chloro-3-[3-[[(4,4-difluoro-5-oxo-pyrrolidin-2-yl)methylamino]methyl]-4-oxo-pyrido[1,2-a]pyrimidin-8-yl]phenyl]phenyl]-2-methoxy-3-pyridyl]methyl]-N-[[(2S)-5-oxopyrrolidin-2-yl]methyl]carbamate (31 mg, 87.30%). MS: m/z found 862.3 [M+H]⁺.

### 8-[2-Chloro-3-[2-chloro-3-[6-methoxy-5-[[[(2S)-5-oxopyrrolidin-2-yl]methylamino]methyl]-2-pyridyl]phenyl]phenyl]-3-[[(4,4-difluoro-5-oxo-pyrrolidin-2-yl)methylamino]methyl]pyrido[1,2-a]pyrimidin-4-one

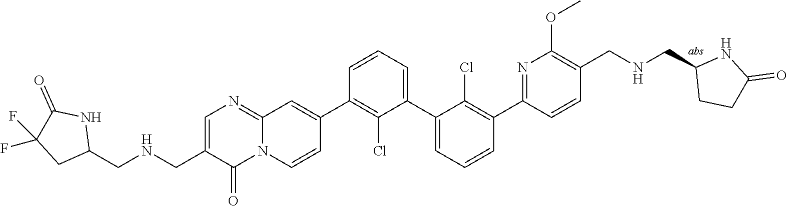 [SMILES: COc1nc(-c2cccc(-c3cccc(-c4ccn5c(=O)c(CNCC6CC(F)(F)C(=O)N6)cnc5c4)c3Cl)c2Cl)ccc1CNC[C@@H]1CCC(=O)N1]

A solution of tert-butyl N-[[6-[2-chloro-3-[2-chloro-3-[3-[[(4,4-difluoro-5-oxo-pyrrolidin-2-yl)methylamino]methyl]-4-oxo-pyrido[1,2-a]pyrimidin-8-yl]phenyl]phenyl]-2-methoxy-3-pyridyl]methyl]-N-[[(2S)-5-oxopyrrolidin-2-yl]methyl]carbamate (25 mg, 0.03 mmol) in DCM/TFA (2:1) was stirred for 30 minutes, concentrated and purified by reversed phase chromatography to give 8-[2-chloro-3-[2-chloro-3-[6-methoxy-5-[[[(2S)-5-oxopyrrolidin-2-yl]methylamino]methyl]-2-pyridyl]phenyl]phenyl]-3-[[(4,4-difluoro-5-oxo-pyrrolidin-2-yl)methylamino]methyl]pyrido[1,2-a]pyrimidin-4-one (21 mg, 95.0%). MS: m/z found 762.2 [M+H]⁺. ¹H NMR (400 MHz, Methanol-d₄): δ 9.15 (dt, J=7.4, 1.0 Hz, 1H), 8.46-8.39 (m, 2H), 7.81 (s, 1H), 7.80-7.76 (m, 1H), 7.69-7.65 (m, 1H), 7.65-7.58 (m, 2H), 7.56-7.51 (m, 3H), 7.51-7.49 (m, 1H), 7.42 (dt, J=7.6, 1.4 Hz, 1H), 7.30 (dd, J=7.4, 1.1 Hz, 1H), 4.05 (s, 3H), 4.03 (d, J=3.3 Hz, 2H), 3.95-3.86 (m, 4H), 2.97-2.82 (m, 3H), 2.73 (ddt, J=22.3, 11.1, 7.2 Hz, 2H), 2.43-2.28 (m, 4H), 1.92-1.80 (m, 1H).

### Example 256: 3-[[(1-Acetyl-4-piperidyl)amino]methyl]-8-[2-chloro-3-[2-chloro-3-[6-methoxy-5-[[[(2S)-5-oxopyrrolidin-2-yl]methylamino]methyl]-2-pyridyl]phenyl]phenyl]pyrido[1,2-a]pyrimidin-4-one

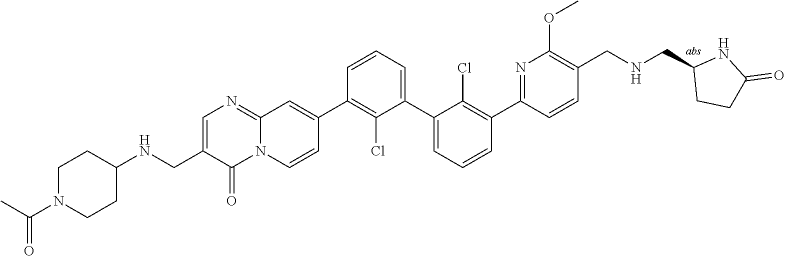 [SMILES: COc1nc(-c2cccc(-c3cccc(-c4ccn5c(=O)c(CNC6CCN(C(C)=O)CC6)cnc5c4)c3Cl)c2Cl)ccc1CNC[C@@H]1CCC(=O)N1]

### tert-Butyl N-[[6-[3-[3-[3-[[(1-acetyl-4-piperidyl)amino]methyl]-4-oxo-pyrido[1,2-a]pyrimidin-8-yl]-2-chloro-phenyl]-2-chloro-phenyl]-2-methoxy-3-pyridyl]methyl]-N-[[(2S)-5-oxopyrrolidin-2-yl]methyl]carbamate

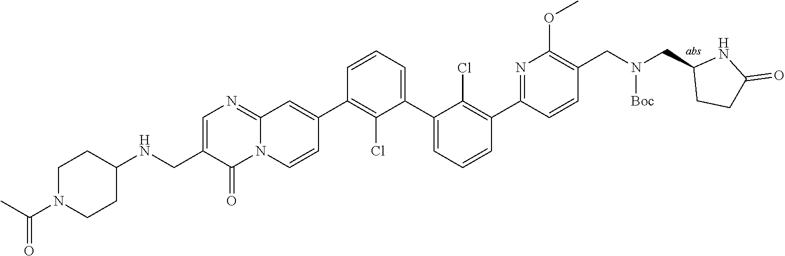 [SMILES: COc1nc(-c2cccc(-c3cccc(-c4ccn5c(=O)c(CNC6CCN(C(C)=O)CC6)cnc5c4)c3Cl)c2Cl)ccc1CN(C[C@@H]1CCC(=O)N1)C(=O)OC(C)(C)C]

A mixture of tert-butyl N-[[6-[2-chloro-3-[2-chloro-3-(3-formyl-4-oxo-pyrido[1,2-a]pyrimidin-8-yl)phenyl]phenyl]-2-methoxy-3-pyridyl]methyl]-N-[[(2S)-5-oxopyrrolidin-2-yl]methyl]carbamate (30 mg, 0.04 mmol), 1-(4-amino-1-piperidyl)ethanone (12 mg, 0.08 mmol) and acetic acid (2.5 uL, 2.5 mg, 0.04 mmol) in MeOH/THF (2:1) was stirred for 2 hours at rt. NaBH₃CN (5 mg, 0.08 mmol) was added, the mixture was stirred for 2 hours, quenched with water, and directly purified by reversed phase chromatography to give tert-butyl N-[[6-[3-[3-[3-[[(1-acetyl-4-piperidyl)amino]methyl]-4-oxo-pyrido[1,2-a]pyrimidin-8-yl]-2-chloro-phenyl]-2-chloro-phenyl]-2-methoxy-3-pyridyl]methyl]-N-[[(2S)-5-oxopyrrolidin-2-yl]methyl]carbamate (31 mg, 88.1%). MS: m/z found 854.3 [M+H]⁺.

### 3-[[(1-Acetyl-4-piperidyl)amino]methyl]-8-[2-chloro-3-[2-chloro-3-[6-methoxy-5-[[[(2S)-5-oxopyrrolidin-2-yl]methylamino]methyl]-2-pyridyl]phenyl]phenyl]pyrido[1,2-a]pyrimidin-4-one

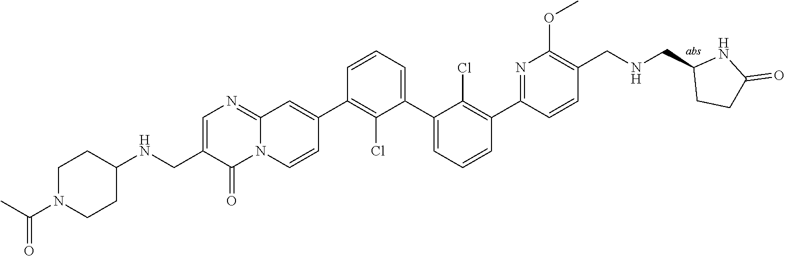 [SMILES: COc1nc(-c2cccc(-c3cccc(-c4ccn5c(=O)c(CNC6CCN(C(C)=O)CC6)cnc5c4)c3Cl)c2Cl)ccc1CNC[C@@H]1CCC(=O)N1]

A solution of tert-butyl N-[[6-[3-[3-[3-[[(1-acetyl-4-piperidyl)amino]methyl]-4-oxo-pyrido[1,2-a]pyrimidin-8-yl]-2-chloro-phenyl]-2-chloro-phenyl]-2-methoxy-3-pyridyl]methyl]-N-[[(2S)-5-oxopyrrolidin-2-yl]methyl]carbamate (25 mg, 0.03 mmol) in DCM/TFA (2:1) was stirred for 30 minutes, concentrated and purified by reversed phase chromatography to give 3-[[(1-acetyl-4-piperidyl)amino]methyl]-8-[2-chloro-3-[2-chloro-3-[6-methoxy-5-[[[(2S)-5-oxopyrrolidin-2-yl]methylamino]methyl]-2-pyridyl]phenyl]phenyl]pyrido[1,2-a]pyrimidin-4-one (21 mg, 95.1%). MS: m/z found 754.3 [M+H]⁺. ¹H NMR (400 MHz, Methanol-d₄): δ 9.17 (d, J=7.4 Hz, 1H), 8.51 (s, 1H), 8.49 (s, 1H), 7.82 (d, J=1.9 Hz, 1H), 7.75 (d, J=7.5 Hz, 1H), 7.67 (dd, J=7.8, 1.8 Hz, 1H), 7.65-7.57 (m, 3H), 7.55-7.49 (m, 2H), 7.40 (dd, J=7.6, 1.8 Hz, 1H), 7.26 (d, J=7.4 Hz, 1H), 4.57 (d, J=13.5 Hz, 1H), 4.12 (s, 2H), 4.03 (s, 3H), 4.01-3.97 (m, 1H), 3.93-3.82 (m, 3H), 3.23-3.11 (m, 2H), 2.80-2.65 (m, 3H), 2.40-2.24 (m, 3H), 2.18 (d, J=18.2 Hz, 2H), 2.12 (s, 3H), 1.89-1.77 (m, 1H), 1.47 (dqd, J=41.0, 12.1, 4.2 Hz, 2H).

### Example 259: 1-[[8-[2-Chloro-3-[2-chloro-3-[6-methoxy-5-[[[(2S)-5-oxopyrrolidin-2-yl]methylamino]methyl]-2-pyridyl]phenyl]phenyl]-4-oxo-pyrido[1,2-a]pyrimidin-3-yl]methyl]azetidine-2-carboxamide

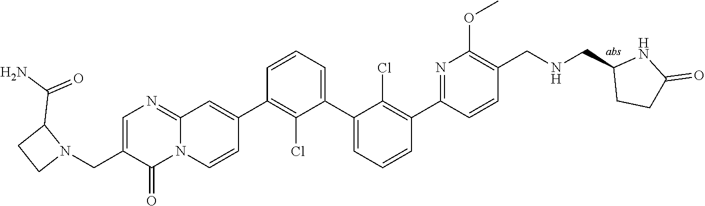 [SMILES: COc1nc(-c2cccc(-c3cccc(-c4ccn5c(=O)c(CN6CCC6C(N)=O)cnc5c4)c3Cl)c2Cl)ccc1CNC[C@@H]1CCC(=O)N1]

### tert-Butyl N-[[6-[3-[3-[3-[(2-carbamoylazetidin-1-yl)methyl]-4-oxo-pyrido[1,2-a]pyrimidin-8-yl]-2-chloro-phenyl]-2-chloro-phenyl]-2-methoxy-3-pyridyl]methyl]-N-[[(2S)-5-oxopyrrolidin-2-yl]methyl]carbamate

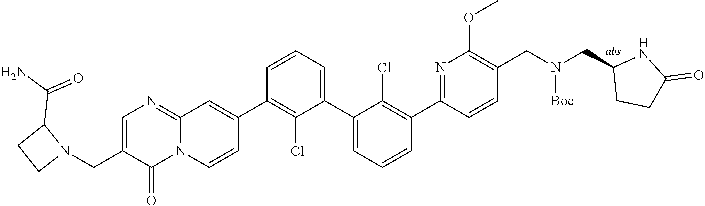 [SMILES: COc1nc(-c2cccc(-c3cccc(-c4ccn5c(=O)c(CN6CCC6C(N)=O)cnc5c4)c3Cl)c2Cl)ccc1CN(C[C@@H]1CCC(=O)N1)C(=O)OC(C)(C)C]

A mixture of tert-butyl N-[[6-[2-chloro-3-[2-chloro-3-(3-formyl-4-oxo-pyrido[1,2-a]pyrimidin-8-yl)phenyl]phenyl]-2-methoxy-3-pyridyl]methyl]-N-[[(2S)-5-oxopyrrolidin-2-yl]methyl]carbamate (30 mg, 0.04 mmol), azetidine-2-carboxamide (8 mg, 0.08 mmol) and acetic acid (2.5 uL, 2.5 mg, 0.04 mmol) in MeOH/THF (2:1) was stirred for 2 hours at rt. NaBH₃CN (5 mg, 0.08 mmol) was added, the mixture was stirred for 2 hours, quenched with water, and directly purified by reversed phase chromatography to give tert-butyl N-[[6-[3-[3-[3-[(2-carbamoylazetidin-1-yl)methyl]-4-oxo-pyrido[1,2-a]pyrimidin-8-yl]-2-chloro-phenyl]-2-chloro-phenyl]-2-methoxy-3-pyridyl]methyl]-N-[[(2S)-5-oxopyrrolidin-2-yl]methyl]carbamate (25 mg, 74.7%). MS: m/z found 812.3 [M+H]⁺.

### 1-[[8-[2-Chloro-3-[2-chloro-3-[6-methoxy-5-[[[(2S)-5-oxopyrrolidin-2-yl]methylamino]methyl]-2-pyridyl]phenyl]phenyl]-4-oxo-pyrido[1,2-a]pyrimidin-3-yl]methyl]azetidine-2-carboxamide

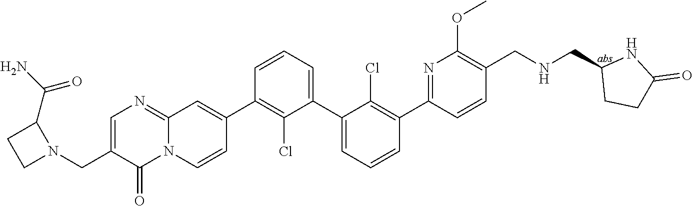 [SMILES: COc1nc(-c2cccc(-c3cccc(-c4ccn5c(=O)c(CN6CCC6C(N)=O)cnc5c4)c3Cl)c2Cl)ccc1CNC[C@@H]1CCC(=O)N1]

A solution of tert-butyl N-[[6-[3-[3-[3-[(2-carbamoylazetidin-1-yl)methyl]-4-oxo-pyrido[1,2-a]pyrimidin-8-yl]-2-chloro-phenyl]-2-chloro-phenyl]-2-methoxy-3-pyridyl]methyl]-N-[[(2S)-5-oxopyrrolidin-2-yl]methyl]carbamate (20 mg, 0.02 mmol) in DCM/TFA (2:1) was stirred for 30 minutes, concentrated and purified by reversed phase chromatography to give 1-[[8-[2-chloro-3-[2-chloro-3-[6-methoxy-5-[[[(2S)-5-oxopyrrolidin-2-yl]methylamino]methyl]-2-pyridyl]phenyl]phenyl]-4-oxo-pyrido[1,2-a]pyrimidin-3-yl]methyl]azetidine-2-carboxamide (17 mg, 96.9%). MS: m/z found 712.2 [M+H]⁺. ¹H NMR (400 MHz, Methanol-d₄): δ 9.14 (d, J=7.4 Hz, 1H), 8.48 (s, 1H), 8.37 (s, 1H), 7.81-7.72 (m, 2H), 7.66 (dd, J=7.7, 1.7 Hz, 1H), 7.65-7.57 (m, 2H), 7.55-7.48 (m, 3H), 7.41 (dd, J=7.6, 1.8 Hz, 1H), 7.28 (d, J=7.5 Hz, 1H), 4.04 (s, 3H), 3.94 (d, J=3.6 Hz, 2H), 3.91-3.77 (m, 3H), 3.62 (d, J=12.9 Hz, 1H), 3.39-3.33 (m, 1H), 3.16 (q, J=8.1 Hz, 1H), 2.88-2.75 (m, 2H), 2.46-2.26 (m, 4H), 2.18-2.07 (m, 1H), 1.89-1.78 (m, 1H).

### Example 260: (2S)-2-[[8-[2-Chloro-3-[2-chloro-3-[6-methoxy-5-[[[(2S)-5-oxopyrrolidin-2-yl]methylamino]methyl]-2-pyridyl]phenyl]phenyl]-4-oxo-pyrido[1,2-a]pyrimidin-3-yl]methylamino]propenamide

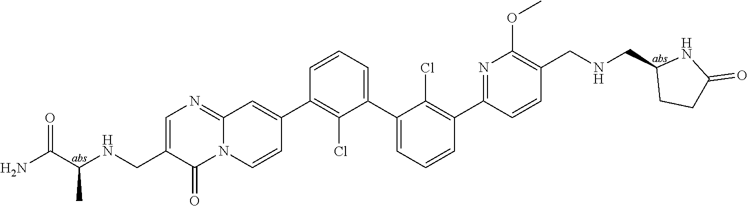 [SMILES: COc1nc(-c2cccc(-c3cccc(-c4ccn5c(=O)c(CN[C@@H](C)C(N)=O)cnc5c4)c3Cl)c2Cl)ccc1CNC[C@@H]1CCC(=O)N1]

### tert-Butyl N-[[6-[3-[3-[3-[[[(1S)-2-amino-1-methyl-2-oxo-ethyl]amino]methyl]-4-oxo-pyrido[1,2-a]pyrimidin-8-yl]-2-chloro-phenyl]-2-chloro-phenyl]-2-methoxy-3-pyridyl]methyl]-N-[[(2S)-5-oxopyrrolidin-2-yl]methyl]carbamate

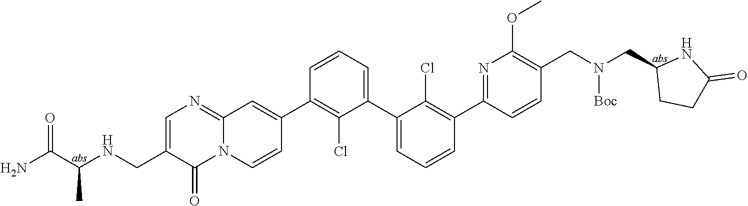 [SMILES: COc1nc(-c2cccc(-c3cccc(-c4ccn5c(=O)c(CN[C@@H](C)C(N)=O)cnc5c4)c3Cl)c2Cl)ccc1CN(C[C@@H]1CCC(=O)N1)C(=O)OC(C)(C)C]

A mixture of tert-butyl N-[[6-[2-chloro-3-[2-chloro-3-(3-formyl-4-oxo-pyrido[1,2-a]pyrimidin-8-yl)phenyl]phenyl]-2-methoxy-3-pyridyl]methyl]-N-[[(2S)-5-oxopyrrolidin-2-yl]methyl]carbamate (30 mg, 0.04 mmol), (2S)-2-aminopropanamide (7 mg, 0.08 mmol) and acetic acid (2.5 uL, 2.5 mg, 0.04 mmol) in MeOH/THF (2:1) was stirred for 2 hours at rt. NaBH₃CN (5 mg, 0.08 mmol) was added, the mixture was stirred for 2 hours, quenched with water, and directly purified by reversed phase chromatography to give tert-butyl N-[[6-[3-[3-[3-[[[(1S)-2-amino-1-methyl-2-oxo-ethyl]amino]methyl]-4-oxo-pyrido[1,2-a]pyrimidin-8-yl]-2-chloro-phenyl]-2-chloro-phenyl]-2-methoxy-3-pyridyl]methyl]-N-[[(2S)-5-oxopyrrolidin-2-yl]methyl]carbamate (27 mg, 81.9%). MS: m/z found 800.3 [M+H]⁺.

### (2S)-2-[[8-[2-Chloro-3-[2-chloro-3-[6-methoxy-5-[[[(2S)-5-oxopyrrolidin-2-yl]methylamino]methyl]-2-pyridyl]phenyl]phenyl]-4-oxo-pyrido[1,2-a]pyrimidin-3-yl]methylamino]propenamide

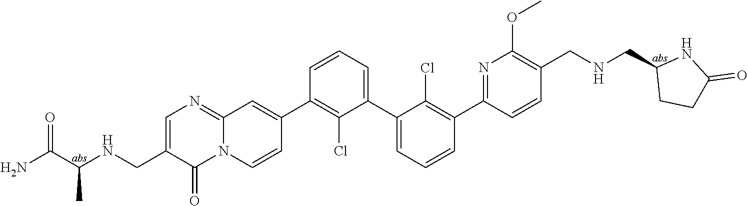 [SMILES: COc1nc(-c2cccc(-c3cccc(-c4ccn5c(=O)c(CN[C@@H](C)C(N)=O)cnc5c4)c3Cl)c2Cl)ccc1CNC[C@@H]1CCC(=O)N1]

A solution of tert-butyl N-[[6-[3-[3-[3-[[[(1S)-2-amino-1-methyl-2-oxo-ethyl]amino]methyl]-4-oxo-pyrido[1,2-a]pyrimidin-8-yl]-2-chloro-phenyl]-2-chloro-phenyl]-2-methoxy-3-pyridyl]methyl]-N-[[(2S)-5-oxopyrrolidin-2-yl]methyl]carbamate (25 mg, 0.03 mmol) in DCM/TFA (2:1) was stirred for 30 minutes, concentrated and purified by reversed phase chromatography to give (2S)-2-[[8-[2-chloro-3-[2-chloro-3-[6-methoxy-5-[[[(2S)-5-oxopyrrolidin-2-yl]methylamino]methyl]-2-pyridyl]phenyl]phenyl]-4-oxo-pyrido[1,2-a]pyrimidin-3-yl]methylamino]propenamide (21 mg, 96.0%). MS: m/z found 700.2 [M+H]⁺. ¹H NMR (400 MHz, Methanol-d₄): δ 9.14 (dt, J=7.5, 1.0 Hz, 1H), 8.40 (s, 1H), 7.79-7.74 (m, 2H), 7.66 (dd, J=7.7, 1.8 Hz, 1H), 7.64-7.57 (m, 2H), 7.56-7.48 (m, 3H), 7.40 (dt, J=7.6, 1.8 Hz, 1H), 7.27 (dd, J=7.4, 1.7 Hz, 1H), 4.03 (s, 3H), 3.98-3.82 (m, 5H), 3.48-3.37 (m, 1H), 2.86-2.72 (m, 2H), 2.39-2.23 (m, 3H), 1.92-1.77 (m, 1H), 1.37 (d, J=7.0 Hz, 3H).

### Example 270: (3S)-4-[[8-[2-Chloro-3-[2-chloro-3-[4-fluoro-6-methoxy-5-[[[(2S)-5-oxopyrrolidin-2-yl]methylamino]methyl]-2-pyridyl]phenyl]phenyl]-4-oxo-pyrido[1,2-a]pyrimidin-3-yl]methylamino]-3-hydroxy-butanoic acid

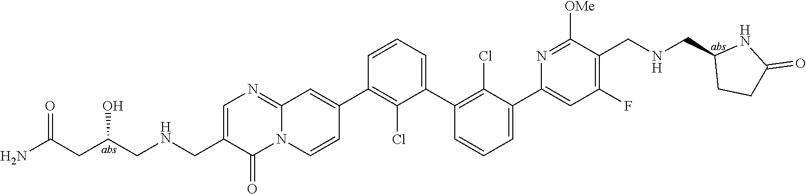 [SMILES: COc1nc(-c2cccc(-c3cccc(-c4ccn5c(=O)c(CNC[C@@H](O)CC(N)=O)cnc5c4)c3Cl)c2Cl)cc(F)c1CNC[C@@H]1CCC(=O)N1]

### tert-Butyl N-[(6-chloro-4-fluoro-2-methoxy-3-pyridyl)methyl]-N-[[(2S)-5-oxopyrrolidin-2-yl]methyl]carbamate

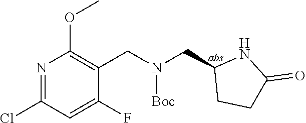 [SMILES: COc1nc(Cl)cc(F)c1CN(C[C@@H]1CCC(=O)N1)C(=O)OC(C)(C)C]

A mixture of 6-chloro-4-fluoro-2-methoxy-pyridine-3-carbaldehyde, (100.00 mg, 0.53 mmol), (5S)-5-(aminomethyl)pyrrolidin-2-one (120 mg, 1.06 mmol) and acetic acid (30 uL, 32 mg, 0.53 mmol) in MeOH/THF (1:1) was stirred for 1 hour at rt. NaBH₃CN (66 mg, 1.06 mmol) was added, the mixture was stirred for additional 30 minutes, quenched with water, and extracted into DCM three times. The combined organic layers were dried over anhydrous sodium sulfate, concentrated, and used in the next step without any further purification. MS: m/z found 288.1 [M+H]⁺.

A mixture of (5S)-5-[[(6-chloro-4-fluoro-2-methoxy-3-pyridyl)methylamino]methyl]pyrrolidin-2-one (140 mg, 0.49 mmol) and di-tert-butyl dicarbonate (117 mg, 0.54 mmol) in dichloromethane was stirred at rt overnight, concentrated and purified on silica gel column (MeOH in DCM, 0 to 10% gradient) to provide tert-butyl N-[(6-chloro-4-fluoro-2-methoxy-3-pyridyl)methyl]-N-[[(2S)-5-oxopyrrolidin-2-yl]methyl]carbamate (180 mg, 95.4%). MS: m/z found 388.1 [M+H]⁺.

### tert-Butyl N-[[6-[2-chloro-3-[2-chloro-3-(3-formyl-4-oxo-pyrido[1,2-a]pyrimidin-8-yl)phenyl]phenyl]-4-fluoro-2-methoxy-3-pyridyl]methyl]-N-[[(2S)-5-oxopyrrolidin-2-yl]methyl]carbamate

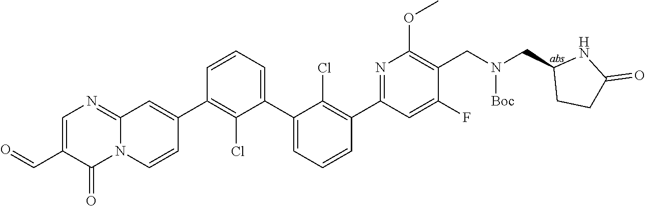 [SMILES: COc1nc(-c2cccc(-c3cccc(-c4ccn5c(=O)c(C=O)cnc5c4)c3Cl)c2Cl)cc(F)c1CN(C[C@@H]1CCC(=O)N1)C(=O)OC(C)(C)C]

To a solution of 8-[2-chloro-3-[2-chloro-3-(4,4,5,5-tetramethyl-1,3,2-dioxaborolan-2-yl)phenyl]phenyl]-4-oxo-pyrido[1,2-a]pyrimidine-3-carbaldehyde (200 mg, 0.38 mmol), tert-butyl N-[(6-chloro-4-fluoro-2-methoxy-3-pyridyl)methyl]-N-[[(2S)-5-oxopyrrolidin-2-yl]methyl]carbamate (149 mg, 0.38 mmol) and potassium carbonate (159 mg, 1.15 mmol) in degassed dioxane/water (6:1) was added Pd(PPh₃)₄ (44 mg, 0.04 mmol) and the mixture was stirred at 90° C. for 4 hours under N₂(g). Water was added and the mixture was extracted into EtOAc three times. The combined organic layers were dried over anhydrous sodium sulfate, concentrated, and purified on silica gel column first using MeOH in DCM (0 to 4% gradient) as eluent to obtain tert-butyl N-[[6-[2-chloro-3-[2-chloro-3-(3-formyl-4-oxo-pyrido[1,2-a]pyrimidin-8-yl)phenyl]phenyl]-4-fluoro-2-methoxy-3-pyridyl]methyl]-N-[[(2S)-5-oxopyrrolidin-2-yl]methyl]carbamate (230 mg, 80.3%). MS: m/z found 746.2 [M+H]⁺.

### (3S)-4-[[8-[3-[3-[5-[[tert-Butoxycarbonyl-[[(2S)-5-oxopyrrolidin-2-yl]methyl]amino]methyl]-4-fluoro-6-methoxy-2-pyridyl]-2-chloro-phenyl]-2-chloro-phenyl]-4-oxo-pyrido[1,2-a]pyrimidin-3-yl]methylamino]-3-hydroxy-butanoic acid

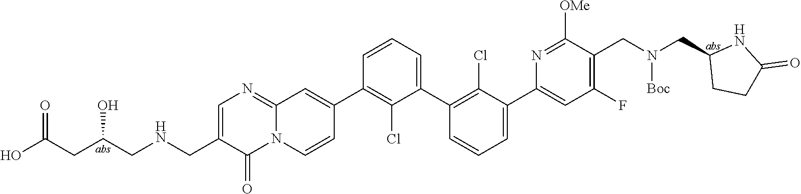 [SMILES: COc1nc(-c2cccc(-c3cccc(-c4ccn5c(=O)c(CNC[C@@H](O)CC(=O)O)cnc5c4)c3Cl)c2Cl)cc(F)c1CN(C[C@@H]1CCC(=O)N1)C(=O)OC(C)(C)C]

A mixture of tert-butyl N-[[6-[2-chloro-3-[2-chloro-3-(3-formyl-4-oxo-pyrido[1,2-a]pyrimidin-8-yl)phenyl]phenyl]-4-fluoro-2-methoxy-3-pyridyl]methyl]-N-[[(2S)-5-oxopyrrolidin-2-yl]methyl]carbamate (15 mg, 0.02 mmol), (3S)-4-amino-3-hydroxy-butanoic acid (5 mg, 0.04 mmol) and acetic acid (1.2 uL, 1.2 mg, 0.02 mmol) in MeOH/THF (2:1) was stirred for 2 hours at rt. NaBH₃CN (2.5 mg, 0.04 mmol) was added, the mixture was stirred for 2 hours, quenched with water, and directly purified by reversed phase chromatography to give (3S)-4-[[8-[3-[3-[5-[[tert-butoxycarbonyl-[[(2S)-5-oxopyrrolidin-2-yl]methyl]amino]methyl]-4-fluoro-6-methoxy-2-pyridyl]-2-chloro-phenyl]-2-chloro-phenyl]-4-oxo-pyrido[1,2-a]pyrimidin-3-yl]methylamino]-3-hydroxy-butanoic acid (14 mg, 82.0%). MS: m/z found 849.3 [M+H]⁺.

### (3S)-4-[[8-[2-Chloro-3-[2-chloro-3-[4-fluoro-6-methoxy-5-[[[(2S)-5-oxopyrrolidin-2-yl]methylamino]methyl]-2-pyridyl]phenyl]phenyl]-4-oxo-pyrido[1,2-a]pyrimidin-3-yl]methylamino]-3-hydroxy-butanoic acid

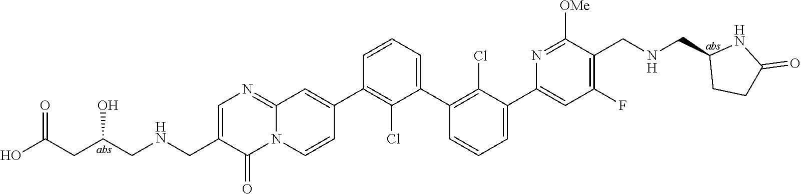 [SMILES: COc1nc(-c2cccc(-c3cccc(-c4ccn5c(=O)c(CNC[C@@H](O)CC(=O)O)cnc5c4)c3Cl)c2Cl)cc(F)c1CNC[C@@H]1CCC(=O)N1]

A solution of (3S)-4-[[8-[3-[3-[5-[[tert-butoxycarbonyl-[[(2S)-5-oxopyrrolidin-2-yl]methyl]amino]methyl]-4-fluoro-6-methoxy-2-pyridyl]-2-chloro-phenyl]-2-chloro-phenyl]-4-oxo-pyrido[1,2-a]pyrimidin-3-yl]methylamino]-3-hydroxy-butanoic acid (15 mg, 0.02 mmol) in DCM/TFA (2:1) was stirred for 30 minutes, concentrated and purified by reversed phase chromatography to give (3S)-4-[[8-[2-chloro-3-[2-chloro-3-[4-fluoro-6-methoxy-5-[[[(2S)-5-oxopyrrolidin-2-yl]methylamino]methyl]-2-pyridyl]phenyl]phenyl]-4-oxo-pyrido[1,2-a]pyrimidin-3-yl]methylamino]-3-hydroxy-butanoic acid (13 mg, 98.2%). MS: m/z found 749.2 [M+H]⁺. ¹H NMR (400 MHz, Methanol-d₄): δ 9.18 (dd, J=7.4, 1.8 Hz, 1H), 8.53 (d, J=1.8 Hz, 1H), 7.85 (d, J=1.9 Hz, 1H), 7.69 (dt, J=7.7, 1.8 Hz, 1H), 7.67-7.59 (m, 3H), 7.56-7.50 (m, 2H), 7.44 (dt, J=7.6, 1.8 Hz, 1H), 7.15 (dd, J=9.2, 1.8 Hz, 1H), 4.31 (s, 2H), 4.21 (ddd, J=8.5, 4.1, 2.2 Hz, 1H), 4.06 (s, 3H), 3.95 (s, 2H), 3.89-3.82 (m, 1H), 3.26 (ddd, J=12.5, 3.9, 1.8 Hz, 1H), 3.06 (ddd, J=12.6, 8.5, 1.8 Hz, 1H), 2.81-2.69 (m, 2H), 2.46 (d, J=6.1 Hz, 2H), 2.37-2.24 (m, 3H), 1.87-1.76 (m, 1H).

### Example 271: Methyl (3S)-4-[[8-[2-chloro-3-[2-chloro-3-[4-fluoro-6-methoxy-5-[[[(2S)-5-oxopyrrolidin-2-yl]methylamino]methyl]-2-pyridyl]phenyl]phenyl]-4-oxo-pyrido[1,2-a]pyrimidin-3-yl]methylamino]-3-hydroxy-butanoate

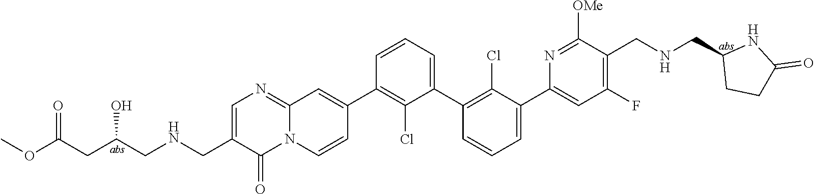 [SMILES: COC(=O)C[C@H](O)CNCc1cnc2cc(-c3cccc(-c4cccc(-c5cc(F)c(CNC[C@@H]6CCC(=O)N6)c(OC)n5)c4Cl)c3Cl)ccn2c1=O]

### Methyl (3S)-4-[[8-[3-[3-[5-[[tert-butoxycarbonyl-[[(2S)-5-oxopyrrolidin-2-yl]methyl]amino]methyl]-4-fluoro-6-methoxy-2-pyridyl]-2-chloro-phenyl]-2-chloro-phenyl]-4-oxo-pyrido[1,2-a]pyrimidin-3-yl]methylamino]-3-hydroxy-butanoate

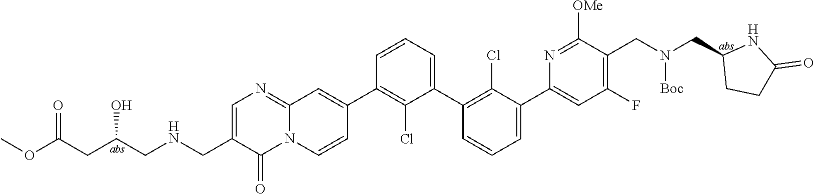 [SMILES: COC(=O)C[C@H](O)CNCc1cnc2cc(-c3cccc(-c4cccc(-c5cc(F)c(CN(C[C@@H]6CCC(=O)N6)C(=O)OC(C)(C)C)c(OC)n5)c4Cl)c3Cl)ccn2c1=O]

A mixture of tert-butyl N-[[6-[2-chloro-3-[2-chloro-3-(3-formyl-4-oxo-pyrido[1,2-a]pyrimidin-8-yl)phenyl]phenyl]-4-fluoro-2-methoxy-3-pyridyl]methyl]-N-[[(2S)-5-oxopyrrolidin-2-yl]methyl]carbamate (30 mg, 0.04 mmol), methyl (3S)-4-amino-3-hydroxy-butanoate (11 mg, 0.08 mmol) and acetic acid (2.30 uL, 2.41 mg, 0.04 mmol) in MeOH/THF (2:1) was stirred for 2 hours at rt. NaBH₃CN (5 mg, 0.08 mmol) was added, the mixture was stirred for 2 hours, quenched with water, and directly purified by reversed phase chromatography to give methyl (3S)-4-[[8-[3-[3-[5-[[tert-butoxycarbonyl-[[(2S)-5-oxopyrrolidin-2-yl]methyl]amino]methyl]-4-fluoro-6-methoxy-2-pyridyl]-2-chloro-phenyl]-2-chloro-phenyl]-4-oxo-pyrido[1,2-a]pyrimidin-3-yl]methylamino]-3-hydroxy-butanoate (28 mg, 80.7%). MS: m/z found 863.3 [M+H]⁺.

### Methyl (3S)-4-[[8-[2-chloro-3-[2-chloro-3-[4-fluoro-6-methoxy-5-[[[(2S)-5-oxopyrrolidin-2-yl]methylamino]methyl]-2-pyridyl]phenyl]phenyl]-4-oxo-pyrido[1,2-a]pyrimidin-3-yl]methylamino]-3-hydroxy-butanoate

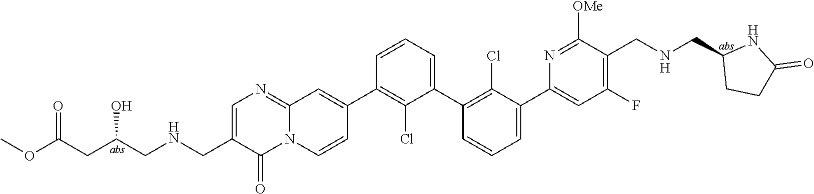 [SMILES: COC(=O)C[C@H](O)CNCc1cnc2cc(-c3cccc(-c4cccc(-c5cc(F)c(CNC[C@@H]6CCC(=O)N6)c(OC)n5)c4Cl)c3Cl)ccn2c1=O]

A solution of methyl (3 S)-4-[[8-[3-[3-[5-[[tert-butoxycarbonyl-[[(2S)-5-oxopyrrolidin-2-yl]methyl]amino]methyl]-4-fluoro-6-methoxy-2-pyridyl]-2-chloro-phenyl]-2-chloro-phenyl]-4-oxo-pyrido[1,2-a]pyrimidin-3-yl]methylamino]-3-hydroxy-butanoate (20 mg, 0.02 mmol) in DCM/TFA (2:1) was stirred for 30 minutes, concentrated and purified by reversed phase chromatography to give methyl (3S)-4-[[8-[2-chloro-3-[2-chloro-3-[4-fluoro-6-methoxy-5-[[[(2S)-5-oxopyrrolidin-2-yl]methylamino]methyl]-2-pyridyl]phenyl]phenyl]-4-oxo-pyrido[1,2-a]pyrimidin-3-yl]methylamino]-3-hydroxy-butanoate (17 mg, 96.1%). MS: m/z found 763.2 [M+H]⁺. ¹H NMR (400 MHz, Methanol-d₄): δ 9.16 (dd, J=7.4, 0.8 Hz, 1H), 8.46 (s, 1H), 7.82 (dt, J=1.9, 0.9 Hz, 1H), 7.69 (dt, J=7.7, 1.4 Hz, 1H), 7.66-7.57 (m, 3H), 7.56-7.51 (m, 2H), 7.43 (dd, J=7.6, 1.8 Hz, 1H), 7.13 (d, J=9.2 Hz, 1H), 4.30-4.23 (m, 1H), 4.15-4.06 (m, 2H), 4.05 (s, 3H), 3.89 (s, 2H), 3.87-3.79 (m, 1H), 3.68 (s, 3H), 3.00 (dd, J=12.4, 3.5 Hz, 1H), 2.86 (dd, J=12.4, 9.1 Hz, 1H), 2.74-2.63 (m, 2H), 2.62-2.47 (m, 2H), 2.36-2.21 (m, 3H), 1.86-1.74 (m, 1H).

### Example 272: 8-[2-Chloro-3-[2-chloro-3-[4-fluoro-6-methoxy-5-[[[(2S)-5-oxopyrrolidin-2-yl]methylamino]methyl]-2-pyridyl]phenyl]phenyl]-3-[[[(2S)-2-hydroxypropyl]amino]methyl]pyrido[1,2-a]pyrimidin-4-one

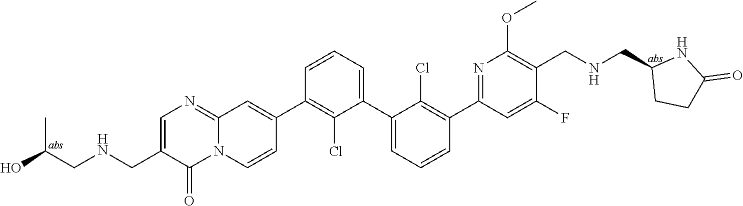 [SMILES: COc1nc(-c2cccc(-c3cccc(-c4ccn5c(=O)c(CNC[C@H](C)O)cnc5c4)c3Cl)c2Cl)cc(F)c1CNC[C@@H]1CCC(=O)N1]

### tert-Butyl N-[[6-[2-chloro-3-[2-chloro-3-[3-[[[(2S)-2-hydroxypropyl]amino]methyl]-4-oxo-pyrido[1,2-a]pyrimidin-8-yl]phenyl]phenyl]-4-fluoro-2-methoxy-3-pyridyl]methyl]-N-[[(2S)-5-oxopyrrolidin-2-yl]methyl]carbamate

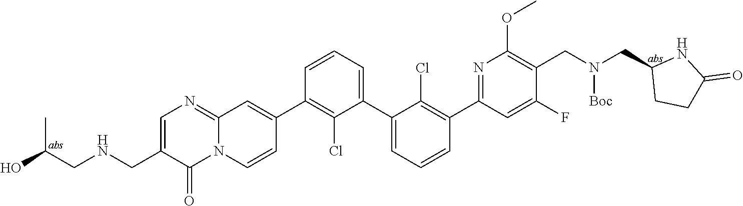 [SMILES: COc1nc(-c2cccc(-c3cccc(-c4ccn5c(=O)c(CNC[C@H](C)O)cnc5c4)c3Cl)c2Cl)cc(F)c1CN(C[C@@H]1CCC(=O)N1)C(=O)OC(C)(C)C]

A mixture of tert-butyl N-[[6-[2-chloro-3-[2-chloro-3-(3-formyl-4-oxo-pyrido[1,2-a]pyrimidin-8-yl)phenyl]phenyl]-4-fluoro-2-methoxy-3-pyridyl]methyl]-N-[[(2S)-5-oxopyrrolidin-2-yl]methyl]carbamate (30 mg, 0.04 mmol), (2S)-1-aminopropan-2-ol (6 mg, 0.08 mmol) and acetic acid (2.3 uL, 2.4 mg, 0.04 mmol) in MeOH/THF (2:1) was stirred for 2 hours at rt. NaBH₃CN (5.05 mg, 0.08 mmol) was added, the mixture was stirred for 2 hours, quenched with water, and directly purified by reversed phase chromatography to give tert-butyl N-[[6-[2-chloro-3-[2-chloro-3-[3-[[[(2S)-2-hydroxypropyl]amino]methyl]-4-oxo-pyrido[1,2-a]pyrimidin-8-yl]phenyl]phenyl]-4-fluoro-2-methoxy-3-pyridyl]methyl]-N-[[(2S)-5-oxopyrrolidin-2-yl]methyl]carbamate (25 mg, 77.2%). MS: m/z found 805.3 [M+H]⁺.

### 8-[2-Chloro-3-[2-chloro-3-[4-fluoro-6-methoxy-5-[[[(2S)-5-oxopyrrolidin-2-yl]methylamino]methyl]-2-pyridyl]phenyl]phenyl]-3-[[[(2S)-2-hydroxypropyl]amino]methyl]pyrido[1,2-a]pyrimidin-4-one

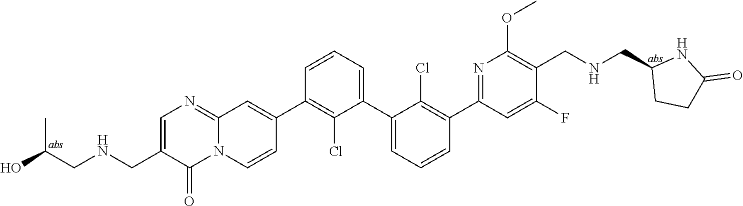 [SMILES: COc1nc(-c2cccc(-c3cccc(-c4ccn5c(=O)c(CNC[C@H](C)O)cnc5c4)c3Cl)c2Cl)cc(F)c1CNC[C@@H]1CCC(=O)N1]

A solution of tert-butyl N-[[6-[2-chloro-3-[2-chloro-3-[3-[[[(2S)-2-hydroxypropyl]amino]methyl]-4-oxo-pyrido[1,2-a]pyrimidin-8-yl]phenyl]phenyl]-4-fluoro-2-methoxy-3-pyridyl]methyl]-N-[[(2S)-5-oxopyrrolidin-2-yl]methyl]carbamate (15 mg, 0.02 mmol) in DCM/TFA (2:1) was stirred for 30 minutes, concentrated and purified by reversed phase chromatography to give 8-[2-chloro-3-[2-chloro-3-[4-fluoro-6-methoxy-5-[[[(2S)-5-oxopyrrolidin-2-yl]methylamino]methyl]-2-pyridyl]phenyl]phenyl]-3-[[[(2S)-2-hydroxypropyl]amino]methyl]pyrido[1,2-a]pyrimidin-4-one (13 mg, 99.0%). MS: m/z found 705.2 [M+H]⁺. ¹H NMR (400 MHz, Methanol-d₄): δ 9.17 (d, J=7.5 Hz, 1H), 8.48 (s, 1H), 7.84 (d, J=1.9 Hz, 1H), 7.69 (dd, J=7.7, 1.8 Hz, 1H), 7.66-7.58 (m, 3H), 7.56-7.50 (m, 2H), 7.44 (dd, J=7.6, 1.7 Hz, 1H), 7.13 (d, J=9.2 Hz, 1H), 4.16 (d, J=3.5 Hz, 2H), 4.05 (s, 3H), 4.03-3.97 (m, 1H), 3.89 (s, 2H), 3.86-3.79 (m, 1H), 3.03-2.95 (m, 1H), 2.81 (dd, J=12.3, 9.4 Hz, 1H), 2.75-2.61 (m, 2H), 2.36-2.22 (m, 3H), 1.86-1.73 (m, 1H), 1.22 (d, J=6.3 Hz, 3H).

### Example 275: (3S)-4-[[8-[2-Chloro-3-[2-chloro-3-[4-fluoro-6-methoxy-5-[[[(2S)-5-oxopyrrolidin-2-yl]methylamino]methyl]-2-pyridyl]phenyl]phenyl]-4-oxo-pyrido[1,2-a]pyrimidin-3-yl]methylamino]-3-hydroxy-butanamide

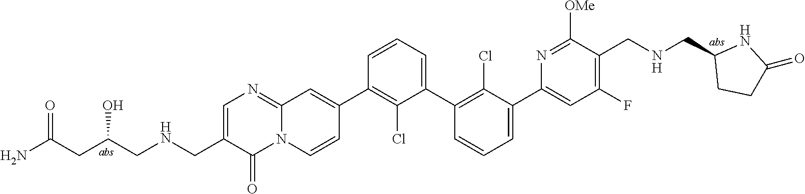 [SMILES: COc1nc(-c2cccc(-c3cccc(-c4ccn5c(=O)c(CNC[C@@H](O)CC(N)=O)cnc5c4)c3Cl)c2Cl)cc(F)c1CNC[C@@H]1CCC(=O)N1]

### tert-Butyl N-[[6-[3-[3-[3-[[[(2S)-4-amino-2-hydroxy-4-oxo-butyl]amino]methyl]-4-oxo-pyrido[1,2-a]pyrimidin-8-yl]-2-chloro-phenyl]-2-chloro-phenyl]-4-fluoro-2-methoxy-3-pyridyl]methyl]-N-[[(2S)-5-oxopyrrolidin-2-yl]methyl]carbamate

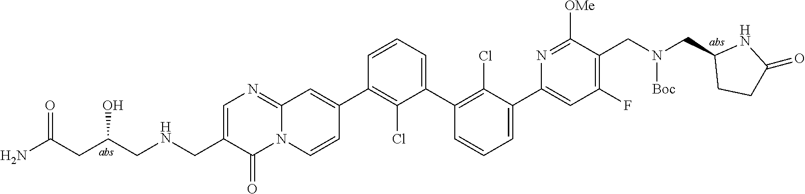 [SMILES: COc1nc(-c2cccc(-c3cccc(-c4ccn5c(=O)c(CNC[C@@H](O)CC(N)=O)cnc5c4)c3Cl)c2Cl)cc(F)c1CN(C[C@@H]1CCC(=O)N1)C(=O)OC(C)(C)C]

A mixture of tert-butyl N-[[6-[2-chloro-3-[2-chloro-3-(3-formyl-4-oxo-pyrido[1,2-a]pyrimidin-8-yl)phenyl]phenyl]-4-fluoro-2-methoxy-3-pyridyl]methyl]-N-[[(2S)-5-oxopyrrolidin-2-yl]methyl]carbamate (30 mg, 0.04 mmol), (3S)-4-amino-3-hydroxy-butanamide (9.5 mg, 0.08 mmol) and acetic acid (2.3 uL, 2.4 mg, 0.04 mmol) in MeOH/THF (2:1) was stirred for 2 hours at rt. NaBH₃CN (5.05 mg, 0.08 mmol) was added, the mixture was stirred for 2 hours, quenched with water, and directly purified by reversed phase chromatography to give tert-butyl N-[[6-[3-[3-[3-[[[(2S)-4-amino-2-hydroxy-4-oxo-butyl]amino]methyl]-4-oxo-pyrido[1,2-a]pyrimidin-8-yl]-2-chloro-phenyl]-2-chloro-phenyl]-4-fluoro-2-methoxy-3-pyridyl]methyl]-N-[[(2S)-5-oxopyrrolidin-2-yl]methyl]carbamate (15 mg, 44.0%). MS: m/z found 848.3 [M+H]⁺.

### (3S)-4-[[8-[2-Chloro-3-[2-chloro-3-[4-fluoro-6-methoxy-5-[[[(2S)-5-oxopyrrolidin-2-yl]methylamino]methyl]-2-pyridyl]phenyl]phenyl]-4-oxo-pyrido[1,2-a]pyrimidin-3-yl]methylamino]-3-hydroxy-butanamide

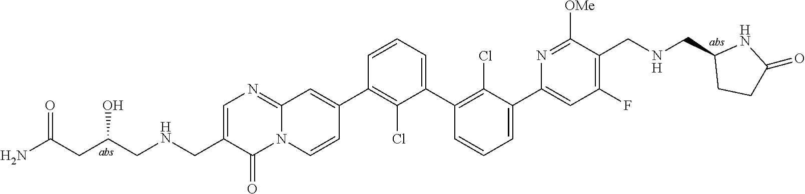 [SMILES: COc1nc(-c2cccc(-c3cccc(-c4ccn5c(=O)c(CNC[C@@H](O)CC(N)=O)cnc5c4)c3Cl)c2Cl)cc(F)c1CNC[C@@H]1CCC(=O)N1]

A solution of tert-butyl N-[[6-[3-[3-[3-[[[(2S)-4-amino-2-hydroxy-4-oxo-butyl]amino]methyl]-4-oxo-pyrido[1,2-a]pyrimidin-8-yl]-2-chloro-phenyl]-2-chloro-phenyl]-4-fluoro-2-methoxy-3-pyridyl]methyl]-N-[[(2S)-5-oxopyrrolidin-2-yl]methyl]carbamate (15 mg, 0.02 mmol) in DCM/TFA (2:1) was stirred for 30 minutes, concentrated and purified by reversed phase chromatography to give (3S)-4-[[8-[2-chloro-3-[2-chloro-3-[4-fluoro-6-methoxy-5-[[[(2S)-5-oxopyrrolidin-2-yl]methylamino]methyl]-2-pyridyl]phenyl]phenyl]-4-oxo-pyrido[1,2-a]pyrimidin-3-yl]methylamino]-3-hydroxy-butanamide (13 mg, 98.3%). MS: m/z found 748.2 [M+H]⁺. ¹H NMR (400 MHz, Methanol-d₄): δ 9.23-9.11 (m, 1H), 8.51 (s, 1H), 7.86 (dt, J=1.9, 0.9 Hz, 1H), 7.72-7.67 (m, 1H), 7.66-7.60 (m, 3H), 7.57-7.50 (m, 2H), 7.44 (dt, J=7.6, 1.4 Hz, 1H), 7.14 (dd, J=9.2, 1.0 Hz, 1H), 4.34-4.27 (m, 1H), 4.27 (s, 2H), 4.06 (s, 3H), 3.94-3.90 (m, 2H), 3.86 (dt, J=12.6, 5.8 Hz, 1H), 3.22 (dd, J=12.7, 3.2 Hz, 1H), 3.02 (dd, J=12.6, 9.4 Hz, 1H), 2.79-2.65 (m, 2H), 2.46 (dd, J=6.2, 2.5 Hz, 2H), 2.38-2.20 (m, 3H), 1.87-1.73 (m, 1H).

### Example 292: (3S)-4-[[8-[2-Chloro-3-[2-chloro-3-[6-methoxy-5-[[[(2S)-5-oxopyrrolidin-2-yl]methylamino]methyl]-2-pyridyl]phenyl]phenyl]-4-oxo-pyrido[1,2-a]pyrimidin-3-yl]methylamino]-3-hydroxy-N-methyl-butanamide

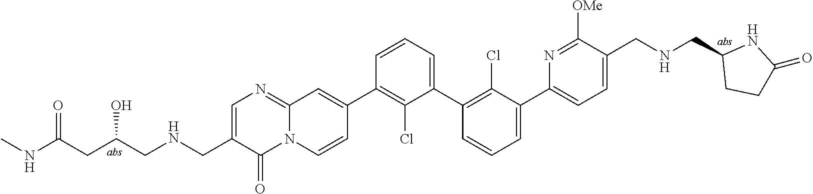 [SMILES: CNC(=O)C[C@H](O)CNCc1cnc2cc(-c3cccc(-c4cccc(-c5ccc(CNC[C@@H]6CCC(=O)N6)c(OC)n5)c4Cl)c3Cl)ccn2c1=O]

### tert-Butyl N-[[6-[2-chloro-3-[2-chloro-3-[3-[[[(2S)-2-hydroxy-4-(methylamino)-4-oxo-butyl]amino]methyl]-4-oxo-pyrido[1,2-a]pyrimidin-8-yl]phenyl]phenyl]-2-methoxy-3-pyridyl]methyl]-N-[[(2S)-5-oxopyrrolidin-2-yl]methyl]carbamate

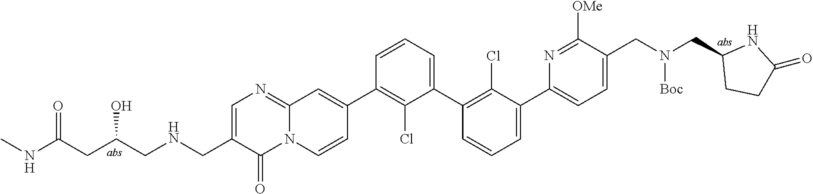 [SMILES: CNC(=O)C[C@H](O)CNCc1cnc2cc(-c3cccc(-c4cccc(-c5ccc(CN(C[C@@H]6CCC(=O)N6)C(=O)OC(C)(C)C)c(OC)n5)c4Cl)c3Cl)ccn2c1=O]

A mixture of tert-butyl N-[[6-[2-chloro-3-[2-chloro-3-(3-formyl-4-oxo-pyrido[1,2-a]pyrimidin-8-yl)phenyl]phenyl]-2-methoxy-3-pyridyl]methyl]-N-[[(2S)-5-oxopyrrolidin-2-yl]methyl]carbamate (32 mg, 0.04 mmol), (3S)-4-amino-3-hydroxy-N-methyl-butanamide (12 mg, 0.09 mmol) and acetic acid (2.5 uL, 2.6 mg, 0.04 mmol) in MeOH/THF (2:1) was stirred for 2 hours at rt. NaBH₃CN (5.5 mg, 0.09 mmol) was added, the mixture was stirred for 2 hours, quenched with water, and purified by reversed phase chromatography to give tert-butyl N-[[6-[2-chloro-3-[2-chloro-3-[3-[[[(2S)-2-hydroxy-4-(methylamino)-4-oxo-butyl]amino]methyl]-4-oxo-pyrido[1,2-a]pyrimidin-8-yl]phenyl]phenyl]-2-methoxy-3-pyridyl]methyl]-N-[[(2S)-5-oxopyrrolidin-2-yl]methyl]carbamate (32 mg, 86.2%). MS: m/z found 844.3 [M+H]⁺.

### (3S)-4-[[8-[2-Chloro-3-[2-chloro-3-[6-methoxy-5-[[[(2S)-5-oxopyrrolidin-2-yl]methylamino]methyl]-2-pyridyl]phenyl]phenyl]-4-oxo-pyrido[1,2-a]pyrimidin-3-yl]methylamino]-3-hydroxy-N-methyl-butanamide

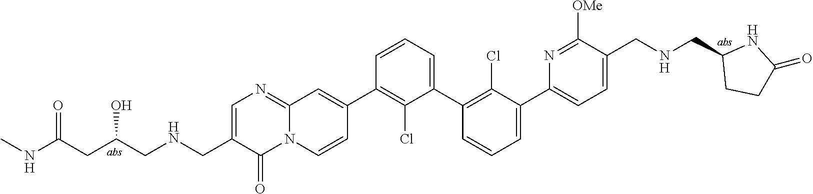 [SMILES: CNC(=O)C[C@H](O)CNCc1cnc2cc(-c3cccc(-c4cccc(-c5ccc(CNC[C@@H]6CCC(=O)N6)c(OC)n5)c4Cl)c3Cl)ccn2c1=O]

A solution of tert-butyl N-[[6-[2-chloro-3-[2-chloro-3-[3-[[[(2S)-2-hydroxy-4-(methylamino)-4-oxo-butyl]amino]methyl]-4-oxo-pyrido[1,2-a]pyrimidin-8-yl]phenyl]phenyl]-2-methoxy-3-pyridyl]methyl]-N-[[(2S)-5-oxopyrrolidin-2-yl]methyl]carbamate (30 mg, 0.04 mmol) in DCM/TFA (2:1) was stirred for 30 minutes, concentrated and purified by reversed phase chromatography to give (3S)-4-[[8-[2-chloro-3-[2-chloro-3-[6-methoxy-5-[[[(2S)-5-oxopyrrolidin-2-yl]methylamino]methyl]-2-pyridyl]phenyl]phenyl]-4-oxo-pyrido[1,2-a]pyrimidin-3-yl]methylamino]-3-hydroxy-N-methyl-butanamide (25 mg, 94.5%). MS: m/z found 744.2 [M+H]⁺. ¹H NMR (400 MHz, Methanol-d₄): δ 9.16 (d, J=7.4 Hz, 1H), 8.53 (s, 1H), 8.47 (s, 1H), 7.82 (dd, J=1.9, 0.9 Hz, 1H), 7.77-7.72 (m, 1H), 7.68-7.65 (m, 1H), 7.65-7.57 (m, 3H), 7.54-7.49 (m, 2H), 7.40 (dd, J=7.6, 1.7 Hz, 1H), 7.25 (d, J=7.4 Hz, 1H), 4.24 (dtd, J=9.7, 6.4, 3.2 Hz, 1H), 4.17-4.07 (m, 2H), 4.02 (s, 3H), 3.91-3.80 (m, 3H), 3.03 (dd, J=12.5, 3.4 Hz, 1H), 2.88 (dd, J=12.4, 8.9 Hz, 1H), 2.79-2.66 (m, 5H), 2.39 (d, J=6.4 Hz, 2H), 2.37-2.25 (m, 3H), 1.87-1.77 (m, 1H).

### Example 293: (3S)-4-[[8-[2-Chloro-3-[2-chloro-3-[4-fluoro-6-methoxy-5-[[[(2S)-5-oxopyrrolidin-2-yl]methylamino]methyl]-2-pyridyl]phenyl]phenyl]-4-oxo-pyrido[1,2-a]pyrimidin-3-yl]methylamino]-3-hydroxy-N-methyl-butanamide

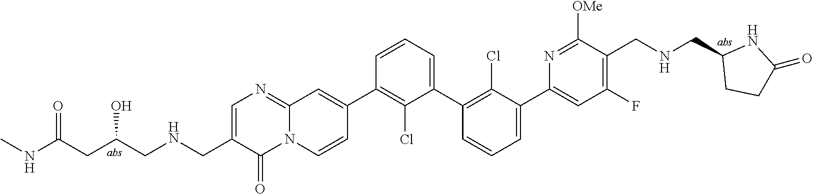 [SMILES: CNC(=O)C[C@H](O)CNCc1cnc2cc(-c3cccc(-c4cccc(-c5cc(F)c(CNC[C@@H]6CCC(=O)N6)c(OC)n5)c4Cl)c3Cl)ccn2c1=O]

### tert-Butyl N-[[6-[2-chloro-3-[2-chloro-3-[3-[[[(2S)-2-hydroxy-4-(methylamino)-4-oxo-butyl]amino]methyl]-4-oxo-pyrido[1,2-a]pyrimidin-8-yl]phenyl]phenyl]-4-fluoro-2-methoxy-3-pyridyl]methyl]-N-[[(2S)-5-oxopyrrolidin-2-yl]methyl]carbamate

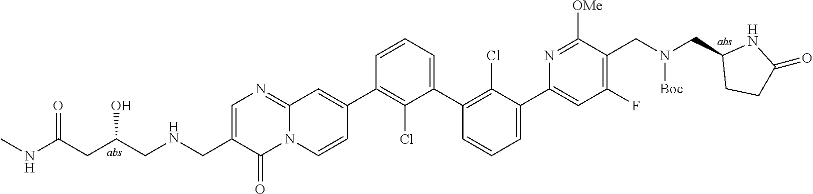 [SMILES: CNC(=O)C[C@H](O)CNCc1cnc2cc(-c3cccc(-c4cccc(-c5cc(F)c(CN(C[C@@H]6CCC(=O)N6)C(=O)OC(C)(C)C)c(OC)n5)c4Cl)c3Cl)ccn2c1=O]

A mixture of tert-butyl N-[[6-[2-chloro-3-[2-chloro-3-(3-formyl-4-oxo-pyrido[1,2-a]pyrimidin-8-yl)phenyl]phenyl]-4-fluoro-2-methoxy-3-pyridyl]methyl]-N-[[(2S)-5-oxopyrrolidin-2-yl]methyl]carbamate (33 mg, 0.04 mmol), (3S)-4-amino-3-hydroxy-N-methyl-butanamide (12 mg, 0.09 mmol) and acetic acid (2.5 uL, 2.6 mg, 0.04 mmol) in MeOH/THF (2:1) was stirred for 2 hours at rt. NaBH₃CN (5.5 mg, 0.09 mmol) was added, the mixture was stirred for 2 hours, quenched with water, and purified by reversed phase chromatography to give tert-butyl N-[[6-[2-chloro-3-[2-chloro-3-[3-[[[(2S)-2-hydroxy-4-(methylamino)-4-oxo-butyl]amino]methyl]-4-oxo-pyrido[1,2-a]pyrimidin-8-yl]phenyl]phenyl]-4-fluoro-2-methoxy-3-pyridyl]methyl]-N-[[(2S)-5-oxopyrrolidin-2-yl]methyl]carbamate (33 mg, 86.5%). MS: m/z found 862.3 [M+H]⁺.

### (3S)-4-[[8-[2-Chloro-3-[2-chloro-3-[4-fluoro-6-methoxy-5-[[[(2S)-5-oxopyrrolidin-2-yl]methylamino]methyl]-2-pyridyl]phenyl]phenyl]-4-oxo-pyrido[1,2-a]pyrimidin-3-yl]methylamino]-3-hydroxy-N-methyl-butanamide

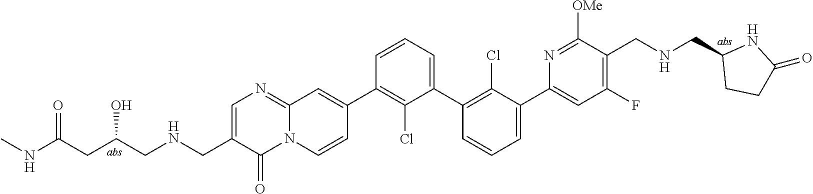 [SMILES: CNC(=O)C[C@H](O)CNCc1cnc2cc(-c3cccc(-c4cccc(-c5cc(F)c(CNC[C@@H]6CCC(=O)N6)c(OC)n5)c4Cl)c3Cl)ccn2c1=O]

A solution of tert-butyl N-[[6-[2-chloro-3-[2-chloro-3-[3-[[[(2S)-2-hydroxy-4-(methylamino)-4-oxo-butyl]amino]methyl]-4-oxo-pyrido[1,2-a]pyrimidin-8-yl]phenyl]phenyl]-4-fluoro-2-methoxy-3-pyridyl]methyl]-N-[[(2S)-5-oxopyrrolidin-2-yl]methyl]carbamate (30 mg, 0.03 mmol) in DCM/TFA (2:1) was stirred for 30 minutes, concentrated and purified by reversed phase chromatography to give (3S)-4-[[8-[2-chloro-3-[2-chloro-3-[4-fluoro-6-methoxy-5-[[[(2S)-5-oxopyrrolidin-2-yl]methylamino]methyl]-2-pyridyl]phenyl]phenyl]-4-oxo-pyrido[1,2-a]pyrimidin-3-yl]methylamino]-3-hydroxy-N-methyl-butanamide (25 mg, 94.3%). MS: m/z found 762.2 [M+H]⁺. ¹H NMR (400 MHz, Methanol-d₄): δ 9.18-9.11 (m, 1H), 8.54 (s, 1H), 8.46 (s, 1H), 7.81 (dd, J=1.8, 0.9 Hz, 1H), 7.69 (dt, J=7.6, 1.5 Hz, 1H), 7.66-7.56 (m, 3H), 7.56-7.50 (m, 2H), 7.43 (dd, J=7.5, 1.6 Hz, 1H), 7.13 (d, J=9.2 Hz, 1H), 4.27-4.16 (m, 1H), 4.12-4.01 (m, 5H), 3.89 (s, 2H), 3.86-3.79 (m, 1H), 2.96 (dd, J=12.4, 3.5 Hz, 1H), 2.82 (dd, J=12.4, 8.7 Hz, 1H), 2.72 (s, 3H), 2.70-2.62 (m, 2H), 2.40-2.21 (m, 5H), 1.86-1.74 (m, 1H).

### Example 297: 8-[2-Chloro-3-[2-chloro-3-[6-methoxy-5-[[[(2S)-5-oxopyrrolidin-2-yl]methylamino]methyl]-2-pyridyl]phenyl]phenyl]-3-[(2,2-difluoroethylamino)methyl]pyrido[1,2-a]pyrimidin-4-one

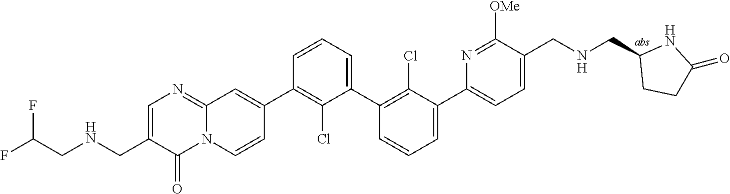 [SMILES: COc1nc(-c2cccc(-c3cccc(-c4ccn5c(=O)c(CNCC(F)F)cnc5c4)c3Cl)c2Cl)ccc1CNC[C@@H]1CCC(=O)N1]

### tert-Butyl N-[[6-[2-chloro-3-[2-chloro-3-[3-[(2,2-difluoroethylamino)methyl]-4-oxo-pyrido[1,2-a]pyrimidin-8-yl]phenyl]phenyl]-2-methoxy-3-pyridyl]methyl]-N-[[(2S)-5-oxopyrrolidin-2-yl]methyl]carbamate

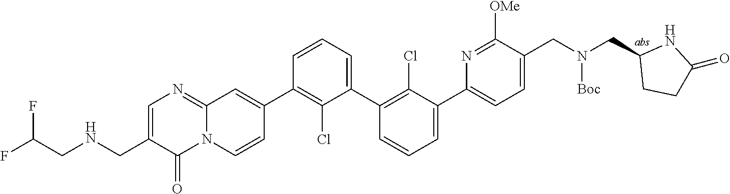 [SMILES: COc1nc(-c2cccc(-c3cccc(-c4ccn5c(=O)c(CNCC(F)F)cnc5c4)c3Cl)c2Cl)ccc1CN(C[C@@H]1CCC(=O)N1)C(=O)OC(C)(C)C]

A mixture of tert-butyl N-[[6-[2-chloro-3-[2-chloro-3-(3-formyl-4-oxo-pyrido[1,2-a]pyrimidin-8-yl)phenyl]phenyl]-2-methoxy-3-pyridyl]methyl]-N-[[(2S)-5-oxopyrrolidin-2-yl]methyl]carbamate (16 mg, 0.02 mmol), 2,2-difluoroethanamine (3.5 mg, 0.04 mmol) and acetic acid (1.3 uL, 1.3 mg, 0.02 mmol) in MeOH/THF (2:1) was stirred for 2 hours at rt. NaBH₃CN (2.8 mg, 0.04 mmol) was added, the mixture was stirred for 2 hours, quenched with water, and purified by reversed phase chromatography to give tert-butyl N-[[6-[2-chloro-3-[2-chloro-3-[3-[(2,2-difluoroethylamino)methyl]-4-oxo-pyrido[1,2-a]pyrimidin-8-yl]phenyl]phenyl]-2-methoxy-3-pyridyl]methyl]-N-[[(2S)-5-oxopyrrolidin-2-yl]methyl]carbamate (15 mg, 86.1%). MS: m/z found 793.2 [M+H]⁺.

### 8-[2-Chloro-3-[2-chloro-3-[6-methoxy-5-[[[(2S)-5-oxopyrrolidin-2-yl]methylamino]methyl]-2-pyridyl]phenyl]phenyl]-3-[(2,2-difluoroethylamino)methyl]pyrido[1,2-a]pyrimidin-4-one

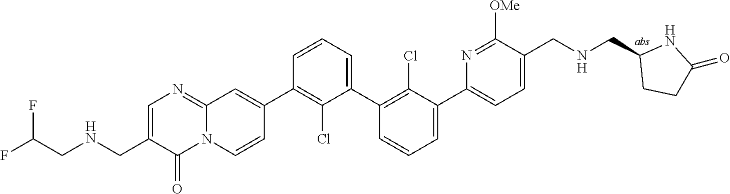 [SMILES: COc1nc(-c2cccc(-c3cccc(-c4ccn5c(=O)c(CNCC(F)F)cnc5c4)c3Cl)c2Cl)ccc1CNC[C@@H]1CCC(=O)N1]

A solution of tert-buty1N-[[6-[2-chloro-3-[2-chloro-3-[3-[(2,2-difluoroethylamino)methyl]-4-oxo-pyrido[1,2-a]pyrimidin-8-yl]phenyl]phenyl]-2-methoxy-3-pyridyl]methyl]-N-[[(2S)-5-oxopyrrolidin-2-yl]methyl]carbamate (25 mg, 0.03 mmol) in DCM/TFA (2:1) was stirred for 30 minutes, concentrated and purified by reversed phase chromatography to give 8-[2-chloro-3-[2-chloro-3-[6-methoxy-5-[[[(2S)-5-oxopyrrolidin-2-yl]methylamino]methyl]-2-pyridyl]phenyl]phenyl]-3-[(2,2-difluoroethylamino)methyl]pyrido[1,2-a]pyrimidin-4-one (21 mg, 96.1%). MS: m/z found 693.2 [M+H]⁺. ¹H NMR (400 MHz, Methanol-d₄): δ 9.12 (dd, J=7.5, 0.8 Hz, 1H), 8.41 (s, 1H), 7.79-7.71 (m, 2H), 7.68-7.64 (m, 1H), 7.64-7.57 (m, 2H), 7.55-7.46 (m, 3H), 7.43-7.37 (m, 1H), 7.25 (d, J=7.5 Hz, 1H), 6.21-5.74 (m, 1H), 4.02 (s, 3H), 3.92-3.82 (m, 5H), 3.00 (td, J=15.5, 4.2 Hz, 2H), 2.80-2.66 (m, 2H), 2.41-2.22 (m, 3H), 1.89-1.77 (m, 1H).

### Example 298: 3-[[[(2S)-2-Aminopropyl]amino]methyl]-8-[2-chloro-3-[2-chloro-3-[6-methoxy-5-[[[(2S)-5-oxopyrrolidin-2-yl]methylamino]methyl]-2-pyridyl]phenyl]phenyl]pyrido[1,2-a]pyrimidin-4-one

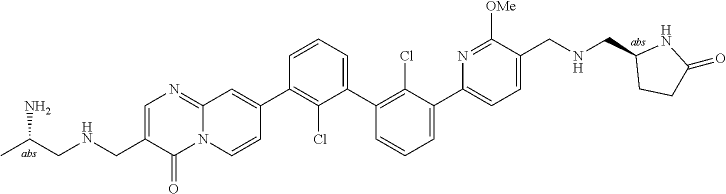 [SMILES: COc1nc(-c2cccc(-c3cccc(-c4ccn5c(=O)c(CNC[C@H](C)N)cnc5c4)c3Cl)c2Cl)ccc1CNC[C@@H]1CCC(=O)N1]

### tert-Butyl N-[[6-[3-[3-[3-[[[(2S)-2-(tert-butoxycarbonylamino)propyl]amino]methyl]-4-oxo-pyrido[1,2-a]pyrimidin-8-yl]-2-chloro-phenyl]-2-chloro-phenyl]-2-methoxy-3-pyridyl]methyl]-N-[[(2S)-5-oxopyrrolidin-2-yl]methyl]carbamate

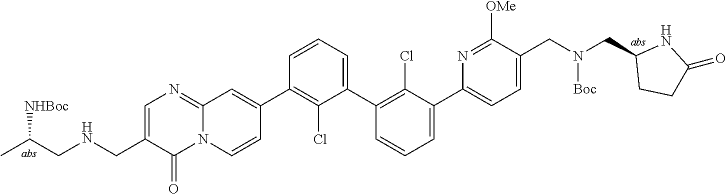 [SMILES: COc1nc(-c2cccc(-c3cccc(-c4ccn5c(=O)c(CNC[C@H](C)NC(=O)OC(C)(C)C)cnc5c4)c3Cl)c2Cl)ccc1CN(C[C@@H]1CCC(=O)N1)C(=O)OC(C)(C)C]

A mixture of tert-butyl N-[[6-[2-chloro-3-[2-chloro-3-(3-formyl-4-oxo-pyrido[1,2-a]pyrimidin-8-yl)phenyl]phenyl]-2-methoxy-3-pyridyl]methyl]-N-[[(2S)-5-oxopyrrolidin-2-yl]methyl]carbamate (32 mg, 0.04 mmol), tert-butyl N-[(1S)-2-amino-1-methyl-ethyl]carbamate (15 mg, 0.09 mmol) and acetic acid (2.5 uL, 2.6 mg, 0.04 mmol) in MeOH/THF (2:1) was stirred for 2 hours at rt. NaBH₃CN (5.5 mg, 0.09 mmol) was added, the mixture was stirred for 2 hours, quenched with water, and purified by reversed phase chromatography to give tert-butyl N-[[6-[3-[3-[3-[[[(2S)-2-(tert-butoxycarbonylamino)propyl]amino]methyl]-4-oxo-pyrido[1,2-a]pyrimidin-8-yl]-2-chloro-phenyl]-2-chloro-phenyl]-2-methoxy-3-pyridyl]methyl]-N-[[(2S)-5-oxopyrrolidin-2-yl]methyl]carbamate (33 mg, 84.7%). MS: m/z found 886.3 [M+H]⁺.

### 3-[[[(2S)-2-Aminopropyl]amino]methyl]-8-[2-chloro-3-[2-chloro-3-[6-methoxy-5-[[[(2S)-5-oxopyrrolidin-2-yl]methylamino]methyl]-2-pyridyl]phenyl]phenyl]pyrido[1,2-a]pyrimidin-4-one

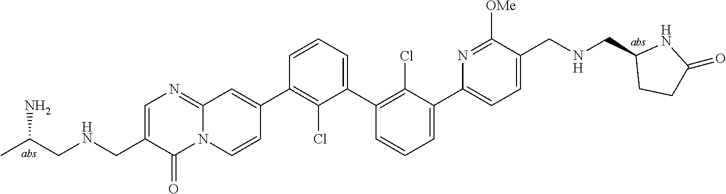 [SMILES: COc1nc(-c2cccc(-c3cccc(-c4ccn5c(=O)c(CNC[C@H](C)N)cnc5c4)c3Cl)c2Cl)ccc1CNC[C@@H]1CCC(=O)N1]

A solution of tert-butyl N-[[6-[3-[3-[3-[[[(2S)-2-(tert-butoxycarbonylamino)propyl]amino]methyl]-4-oxo-pyrido[1,2-a]pyrimidin-8-yl]-2-chloro-phenyl]-2-chloro-phenyl]-2-methoxy-3-pyridyl]methyl]-N-[[(2S)-5-oxopyrrolidin-2-yl]methyl]carbamate (25 mg, 0.03 mmol) in DCM/TFA (2:1) was stirred for 30 minutes, concentrated and purified by reversed phase chromatography to give 3-[[[(2S)-2-aminopropyl]amino]methyl]-8-[2-chloro-3-[2-chloro-3-[6-methoxy-5-[[[(2S)-5-oxopyrrolidin-2-yl]methylamino]methyl]-2-pyridyl]phenyl]phenyl]pyrido[1,2-a]pyrimidin-4-one (19 mg, 98.2%). MS: m/z found 686.2 [M+H]⁺. ¹H NMR (400 MHz, Methanol-d₄): δ 9.15 (dt, J=7.5, 0.9 Hz, 1H), 8.41 (s, 1H), 7.78 (dt, J=1.9, 0.9 Hz, 1H), 7.74 (dd, J=7.5, 0.9 Hz, 1H), 7.68-7.65 (m, 1H), 7.64-7.58 (m, 2H), 7.57-7.48 (m, 3H), 7.40 (ddd, J=7.6, 1.8, 1.0 Hz, 1H), 7.25 (dd, J=7.4, 1.0 Hz, 1H), 4.02 (s, 3H), 3.90-3.79 (m, 5H), 3.40 (ddd, J=9.2, 6.6, 4.1 Hz, 1H), 2.91 (ddd, J=13.0, 4.2, 1.0 Hz, 1H), 2.77-2.68 (m, 2H), 2.67-2.57 (m, 1H), 2.38-2.24 (m, 3H), 1.89-1.76 (m, 1H), 1.28 (dd, J=6.7, 1.0 Hz, 3H).

### Example 302: 8-[2-Chloro-3-[2-chloro-3-[6-methoxy-5-[[[(2S)-5-oxopyrrolidin-2-yl]methylamino]methyl]-2-pyridyl]phenyl]phenyl]-3-[[[(2S)-2-methoxypropyl]amino]methyl]pyrido[1,2-a]pyrimidin-4-one

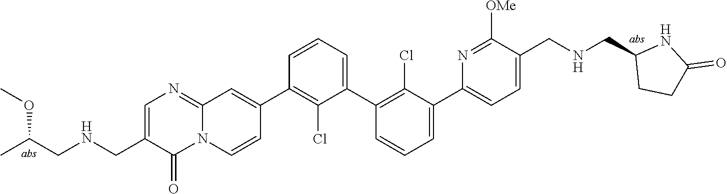 [SMILES: COc1nc(-c2cccc(-c3cccc(-c4ccn5c(=O)c(CNC[C@H](C)OC)cnc5c4)c3Cl)c2Cl)ccc1CNC[C@@H]1CCC(=O)N1]

### tert-Butyl N-[[6-[2-chloro-3-[2-chloro-3-[3-[[[(2S)-2-methoxypropyl]amino]methyl]-4-oxo-pyrido[1,2-a]pyrimidin-8-yl]phenyl]phenyl]-2-methoxy-3-pyridyl]methyl]-N-[[(2S)-5-oxopyrrolidin-2-yl]methyl]carbamate

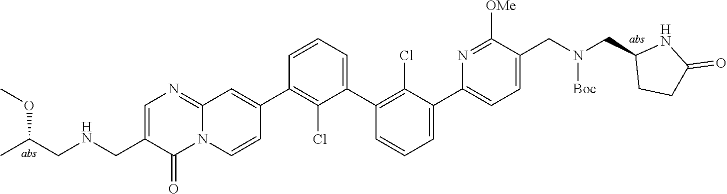 [SMILES: COc1nc(-c2cccc(-c3cccc(-c4ccn5c(=O)c(CNC[C@H](C)OC)cnc5c4)c3Cl)c2Cl)ccc1CN(C[C@@H]1CCC(=O)N1)C(=O)OC(C)(C)C]

A mixture of tert-butyl N-[[6-[2-chloro-3-[2-chloro-3-(3-formyl-4-oxo-pyrido[1,2-a]pyrimidin-8-yl)phenyl]phenyl]-2-methoxy-3-pyridyl]methyl]-N-[[(2S)-5-oxopyrrolidin-2-yl]methyl]carbamate (16 mg, 0.02 mmol), (2S)-2-methoxypropan-1-amine (4 mg, 0.04 mmol) and acetic acid (1.3 uL, 1.3 mg, 0.02 mmol) in MeOH/THF (2:1) was stirred for 2 hours at rt. NaBH₃CN (2.8 mg, 0.04 mmol) was added, the mixture was stirred for 2 hours, quenched with water, and purified by reversed phase chromatography to give tert-butyl N-[[6-[2-chloro-3-[2-chloro-3-[3-[[[(2S)-2-methoxypropyl]amino]methyl]-4-oxo-pyrido[1,2-a]pyrimidin-8-yl]phenyl]phenyl]-2-methoxy-3-pyridyl]methyl]-N-[[(2S)-5-oxopyrrolidin-2-yl]methyl]carbamate (16 mg, 90.9%). MS: m/z found 801.3 [M+H]⁺.

### 8-[2-Chloro-3-[2-chloro-3-[6-methoxy-5-[[[(2S)-5-oxopyrrolidin-2-yl]methylamino]methyl]-2-pyridyl]phenyl]phenyl]-3-[[[(2S)-2-methoxypropyl]amino]methyl]pyrido[1,2-a]pyrimidin-4-one

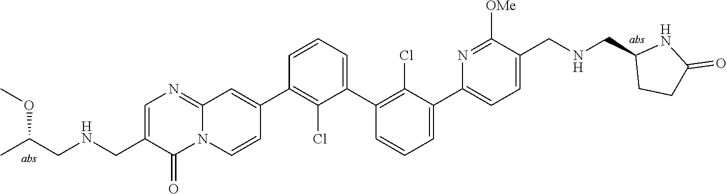 [SMILES: COc1nc(-c2cccc(-c3cccc(-c4ccn5c(=O)c(CNC[C@H](C)OC)cnc5c4)c3Cl)c2Cl)ccc1CNC[C@@H]1CCC(=O)N1]

A solution of tert-butyl N-[[6-[2-chloro-3-[2-chloro-3-[3-[[[(2S)-2-methoxypropyl]amino]methyl]-4-oxo-pyrido[1,2-a]pyrimidin-8-yl]phenyl]phenyl]-2-methoxy-3-pyridyl]methyl]-N-[[(2S)-5-oxopyrrolidin-2-yl]methyl]carbamate (10 mg, 0.01 mmol) in DCM/TFA (2:1) was stirred for 30 minutes, concentrated and purified by reversed phase chromatography to give 8-[2-chloro-3-[2-chloro-3-[6-methoxy-5-[[[(2S)-5-oxopyrrolidin-2-yl]methylamino]methyl]-2-pyridyl]phenyl]phenyl]-3-[[[(2S)-2-methoxypropyl]amino]methyl]pyrido[1,2-a]pyrimidin-4-one (8 mg, 91.4%). MS: m/z found 701.2 [M+H]⁺. ¹H NMR (400 MHz, Methanol-d₄): δ 9.14 (d, J=7.4 Hz, 1H), 8.44 (s, 1H), 7.82-7.77 (m, 1H), 7.74 (d, J=7.5 Hz, 1H), 7.66 (dd, J=7.7, 1.7 Hz, 1H), 7.64-7.55 (m, 3H), 7.54-7.48 (m, 2H), 7.40 (dd, J=7.6, 1.6 Hz, 1H), 7.25 (dd, J=7.4, 1.1 Hz, 1H), 4.05-3.94 (m, 5H), 3.89-3.79 (m, 3H), 3.60 (ddd, J=9.4, 6.2, 3.4 Hz, 1H), 3.37 (s, 3H), 2.88-2.63 (m, 4H), 2.38-2.22 (m, 3H), 1.88-1.76 (m, 1H), 1.17 (d, J=6.1 Hz, 3H).

### Example 303: 8-[2-Chloro-3-[2-chloro-3-[6-methoxy-5-[[[(2S)-5-oxopyrrolidin-2-yl]methylamino]methyl]-2-pyridyl]phenyl]phenyl]-3-[(2,2,2-trifluoroethylamino)methyl]pyrido[1,2-a]pyrimidin-4-one

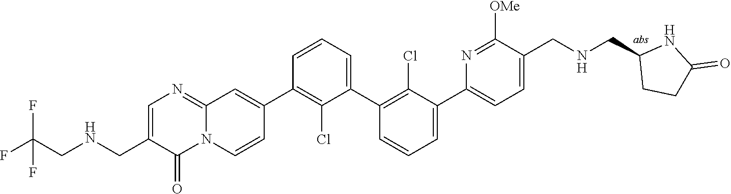 [SMILES: COc1nc(-c2cccc(-c3cccc(-c4ccn5c(=O)c(CNCC(F)(F)F)cnc5c4)c3Cl)c2Cl)ccc1CNC[C@@H]1CCC(=O)N1]

### tert-Butyl N-[[6-[2-chloro-3-[2-chloro-3-[4-oxo-3-[(2,2,2-trifluoroethylamino)methyl]pyrido[1,2-a]pyrimidin-8-yl]phenyl]phenyl]-2-methoxy-3-pyridyl]methyl]-N-[[(2S)-5-oxopyrrolidin-2-yl]methyl]carbamate

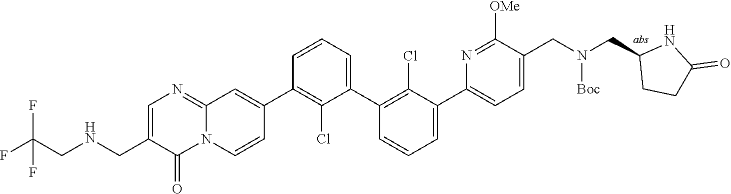 [SMILES: COc1nc(-c2cccc(-c3cccc(-c4ccn5c(=O)c(CNCC(F)(F)F)cnc5c4)c3Cl)c2Cl)ccc1CN(C[C@@H]1CCC(=O)N1)C(=O)OC(C)(C)C]

A mixture of tert-butyl N-[[6-[2-chloro-3-[2-chloro-3-(3-formyl-4-oxo-pyrido[1,2-a]pyrimidin-8-yl)phenyl]phenyl]-2-methoxy-3-pyridyl]methyl]-N-[[(2S)-5-oxopyrrolidin-2-yl]methyl]carbamate (16 mg, 0.02 mmol), 2,2,2-trifluoroethanamine (4.4 mg, 0.04 mmol) and acetic acid (1.3 uL, 1.3 mg, 0.02 mmol) in MeOH/THF (2:1) was stirred for 2 hours at rt. NaBH₃CN (2.8 mg, 0.04 mmol) was added, the mixture was stirred for 2 hours, quenched with water, and purified by reversed phase chromatography to give tert-butyl N-[[6-[2-chloro-3-[2-chloro-3-[4-oxo-3-[(2,2,2-trifluoroethylamino)methyl]pyrido[1,2-a]pyrimidin-8-yl]phenyl]phenyl]-2-methoxy-3-pyridyl]methyl]-N-[[(2S)-5-oxopyrrolidin-2-yl]methyl]carbamate (17 mg, 95.4%). MS: m/z found 793.2 [M+H]⁺.

### 8-[2-Chloro-3-[2-chloro-3-[6-methoxy-5-[[[(2S)-5-oxopyrrolidin-2-yl]methylamino]methyl]-2-pyridyl]phenyl]phenyl]-3-[(2,2,2-trifluoroethylamino)methyl]pyrido[1,2-a]pyrimidin-4-one

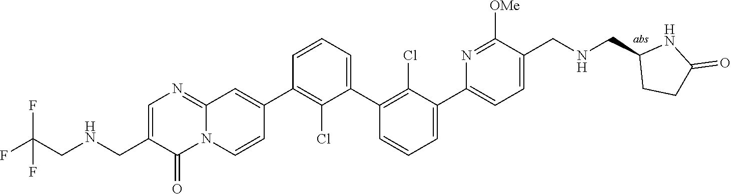 [SMILES: COc1nc(-c2cccc(-c3cccc(-c4ccn5c(=O)c(CNCC(F)(F)F)cnc5c4)c3Cl)c2Cl)ccc1CNC[C@@H]1CCC(=O)N1]

A solution of tert-butyl N-[[6-[2-chloro-3-[2-chloro-3-[4-oxo-3-[(2,2,2-trifluoroethylamino)methyl]pyrido[1,2-a]pyrimidin-8-yl]phenyl]phenyl]-2-methoxy-3-pyridyl]methyl]-N-[[(2S)-5-oxopyrrolidin-2-yl]methyl]carbamate (10 mg, 0.01 mmol) in DCM/TFA (2:1) was stirred for 30 minutes, concentrated and purified by reversed phase chromatography to give 8-[2-chloro-3-[2-chloro-3-[6-methoxy-5-[[[(2S)-5-oxopyrrolidin-2-yl]methylamino]methyl]-2-pyridyl]phenyl]phenyl]-3-[(2,2,2-trifluoroethylamino)methyl]pyrido[1,2-a]pyrimidin-4-one (8 mg, 91.3%). MS: m/z found 711.2 [M+H]⁺. ¹H NMR (400 MHz, Methanol-d₄): δ 9.12 (dd, J=7.4, 0.9 Hz, 1H), 8.41 (s, 1H), 7.79-7.71 (m, 2H), 7.68-7.64 (m, 1H), 7.64-7.57 (m, 2H), 7.51 (ddt, J=8.7, 7.6, 1.6 Hz, 3H), 7.43-7.38 (m, 1H), 7.26 (dd, J=7.4, 0.9 Hz, 1H), 4.02 (s, 3H), 3.92 (s, 2H), 3.89-3.82 (m, 3H), 3.30-3.25 (m, 2H), 2.79-2.68 (m, 2H), 2.38-2.25 (m, 3H), 1.88-1.77 (m, 1H).

### Example 306: 8-[2-Chloro-3-[2-chloro-3-[6-methoxy-5-[[[(2S)-5-oxopyrrolidin-2-yl]methylamino]methyl]-2-pyridyl]phenyl]phenyl]-3-[[[(2S)-oxetan-2-yl]methylamino]methyl]pyrido[1,2-a]pyrimidin-4-one

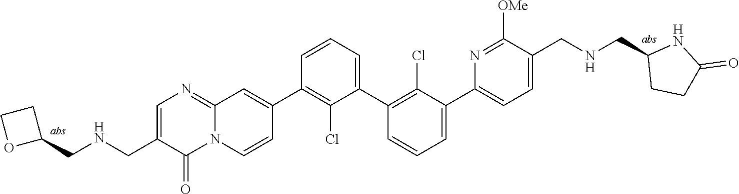 [SMILES: COc1nc(-c2cccc(-c3cccc(-c4ccn5c(=O)c(CNC[C@@H]6CCO6)cnc5c4)c3Cl)c2Cl)ccc1CNC[C@@H]1CCC(=O)N1]

### tert-Butyl N-[[6-[2-chloro-3-[2-chloro-3-[3-[[[(2S)-oxetan-2-yl]methylamino]methyl]-4-oxo-pyrido[1,2-a]pyrimidin-8-yl]phenyl]phenyl]-2-methoxy-3-pyridyl]methyl]-N-[[(2S)-5-oxopyrrolidin-2-yl]methyl]carbamate

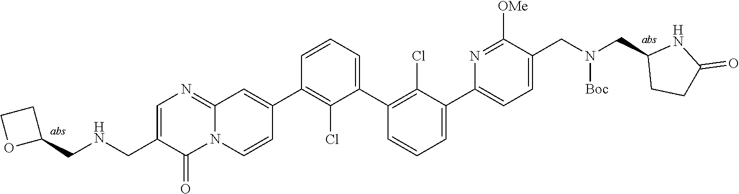 [SMILES: COc1nc(-c2cccc(-c3cccc(-c4ccn5c(=O)c(CNC[C@@H]6CCO6)cnc5c4)c3Cl)c2Cl)ccc1CN(C[C@@H]1CCC(=O)N1)C(=O)OC(C)(C)C]

A mixture of tert-butyl N-[[6-[2-chloro-3-[2-chloro-3-(3-formyl-4-oxo-pyrido[1,2-a]pyrimidin-8-yl)phenyl]phenyl]-2-methoxy-3-pyridyl]methyl]-N-[[(2S)-5-oxopyrrolidin-2-yl]methyl]carbamate (10 mg, 0.01 mmol), [(2S)-oxetan-2-yl]methanamine (2.39 mg, 0.03 mmol) and acetic acid (0.7 uL, 0.8 mg, 0.01 mmol) in MeOH/THF (2:1) was stirred for 2 hours at rt. NaBH₃CN (1.7 mg, 0.03 mmol) was added, the mixture was stirred for 2 hours, quenched with water, and purified by reversed phase chromatography to give tert-butyl N-[[6-[2-chloro-3-[2-chloro-3-[3-[[[(2S)-oxetan-2-yl]methylamino]methyl]-4-oxo-pyrido[1,2-a]pyrimidin-8-yl]phenyl]phenyl]-2-methoxy-3-pyridyl]methyl]-N-[[(2S)-5-oxopyrrolidin-2-yl]methyl]carbamate (10 mg, 91.1%). MS: m/z found 799.3 [M+H]⁺.

### 8-[2-Chloro-3-[2-chloro-3-[6-methoxy-5-[[[(2S)-5-oxopyrrolidin-2-yl]methylamino]methyl]-2-pyridyl]phenyl]phenyl]-3-[[[(2S)-oxetan-2-yl]methylamino]methyl]pyrido[1,2-a]pyrimidin-4-one

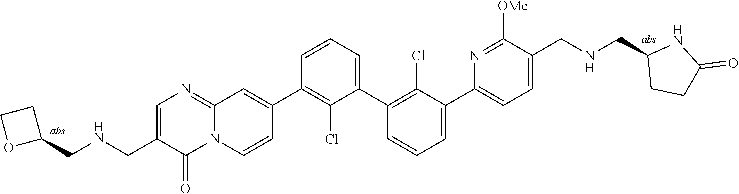 [SMILES: COc1nc(-c2cccc(-c3cccc(-c4ccn5c(=O)c(CNC[C@@H]6CCO6)cnc5c4)c3Cl)c2Cl)ccc1CNC[C@@H]1CCC(=O)N1]

A solution of tert-butyl N-[[6-[2-chloro-3-[2-chloro-3-[3-[[[(2S)-oxetan-2-yl]methylamino]methyl]-4-oxo-pyrido[1,2-a]pyrimidin-8-yl]phenyl]phenyl]-2-methoxy-3-pyridyl]methyl]-N-[[(2S)-5-oxopyrrolidin-2-yl]methyl]carbamate (10 mg, 0.01 mmol) in DCM/TFA (2:1) was stirred for 30 minutes, concentrated and purified by reversed phase chromatography to give 8-[2-chloro-3-[2-chloro-3-[6-methoxy-5-[[[(2S)-5-oxopyrrolidin-2-yl]methylamino]methyl]-2-pyridyl]phenyl]phenyl]-3-[[[(2S)-oxetan-2-yl]methylamino]methyl]pyrido[1,2-a]pyrimidin-4-one (8 mg, 91.4%). MS: m/z found 699.2 [M+H]⁺. ¹H NMR (400 MHz, Methanol-d₄): δ 9.14 (dd, J=7.4, 1.0 Hz, 1H), 8.43 (s, 1H), 7.78 (dd, J=2.0, 1.0 Hz, 1H), 7.74 (dd, J=7.5, 1.3 Hz, 1H), 7.66 (dt, J=7.8, 1.7 Hz, 1H), 7.64-7.57 (m, 2H), 7.57-7.48 (m, 3H), 7.40 (dd, J=7.5, 1.6 Hz, 1H), 7.25 (d, J=7.4 Hz, 1H), 5.01 (qd, J=7.3, 3.9 Hz, 1H), 4.69 (ddd, J=8.6, 7.4, 5.8 Hz, 1H), 4.56 (ddd, J=9.2, 6.7, 5.3 Hz, 1H), 4.02 (s, 3H), 3.96 (s, 2H), 3.89-3.79 (m, 3H), 3.10 (ddd, J=12.8, 7.4, 1.4 Hz, 1H), 2.97-2.86 (m, 1H), 2.80-2.64 (m, 3H), 2.50 (ddt, J=11.0, 8.8, 7.2 Hz, 1H), 2.40-2.22 (m, 3H), 1.88-1.77 (m, 1H).

### Example 307: 8-[2-Chloro-3-[2-chloro-3-[6-methoxy-5-[[[(2S)-5-oxopyrrolidin-2-yl]methylamino]methyl]-2-pyridyl]phenyl]phenyl]-3-[[[(2S)-tetrahydrofuran-2-yl]methylamino]methyl]pyrido[1,2-a]pyrimidin-4-one

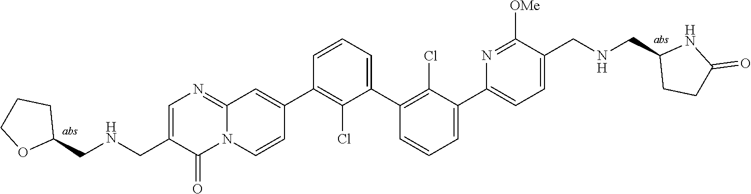 [SMILES: COc1nc(-c2cccc(-c3cccc(-c4ccn5c(=O)c(CNC[C@@H]6CCCO6)cnc5c4)c3Cl)c2Cl)ccc1CNC[C@@H]1CCC(=O)N1]

### tert-Butyl N-[[6-2-chloro-3-[2-chloro-3-[4-oxo-3-1((2S)-tetrahydrofuran-2-yl]methylamino[methyl[pyrido1,2-a]pyrimidin-8-yl]phenyl]phenyll-2-methoxy-3-pyridyl]methyl-N-e(2S)-5-oxopyrrolidin-2-yl]methyl]carbamate

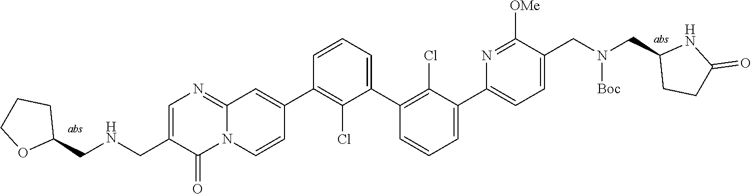 [SMILES: COc1nc(-c2cccc(-c3cccc(-c4ccn5c(=O)c(CNC[C@@H]6CCCO6)cnc5c4)c3Cl)c2Cl)ccc1CN(C[C@@H]1CCC(=O)N1)C(=O)OC(C)(C)C]

A mixture of tert-butyl N-[[6-[2-chloro-3-[2-chloro-3-(3-formyl-4-oxo-pyrido[1,2-a]pyrimidin-8-yl)phenyl]phenyl]-2-methoxy-3-pyridyl]methyl]-N-[[(2S)-5-oxopyrrolidin-2-yl]methyl]carbamate (10 mg, 0.01 mmol), [(2S)-tetrahydrofuran-2-yl]methanamine (2.8 mg, 0.03 mmol) and acetic acid (0.8 uL, 0.8 mg, 0.01 mmol) in MeOH/THF (2:1) was stirred for 2 hours at rt. NaBH₃CN (1.7 mg, 0.03 mmol) was added, the mixture was stirred for 2 hours, quenched with water, and purified by reversed phase chromatography to give tert-butyl N-[[6-[2-chloro-3-[2-chloro-3-[4-oxo-3-[[[(2S)-tetrahydrofuran-2-yl]methylamino]methyl]pyrido[1,2-a]pyrimidin-8-yl]phenyl]phenyl]-2-methoxy-3-pyridyl]methyl]-N-[[(2S)-5-oxopyrrolidin-2-yl]methyl]carbamate (10 mg, 89.5%). MS: m/z found 813.3 [M+H]⁺.

### 8-[2-Chloro-3-[2-chloro-3-[6-methoxy-5-[[[(2S)-5-oxopyrrolidin-2-yl]methylamino]methyl]-2-pyridyl]phenyl]phenyl]-3-[[[(2S)-tetrahydrofuran-2-yl]methylamino]methyl]pyrido[1,2-a]pyrimidin-4-one

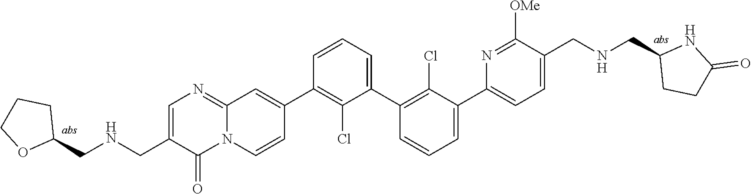 [SMILES: COc1nc(-c2cccc(-c3cccc(-c4ccn5c(=O)c(CNC[C@@H]6CCCO6)cnc5c4)c3Cl)c2Cl)ccc1CNC[C@@H]1CCC(=O)N1]

A solution of tert-butyl N-[[6-[2-chloro-3-[2-chloro-3-[4-oxo-3-[[[(2S)-tetrahydrofuran-2-yl]methylamino]methyl]pyrido[1,2-a]pyrimidin-8-yl]phenyl]phenyl]-2-methoxy-3-pyridyl]methyl]-N-[[(2S)-5-oxopyrrolidin-2-yl]methyl]carbamate (10 mg, 0.01 mmol) in DCM/TFA (2:1) was stirred for 30 minutes, concentrated and purified by reversed phase chromatography to give 8-[2-chloro-3-[2-chloro-3-[6-methoxy-5-[[[(2S)-5-oxopyrrolidin-2-yl]methylamino]methyl]-2-pyridyl]phenyl]phenyl]-3-[[[(2S)-tetrahydrofuran-2-yl]methylamino]methyl]pyrido[1,2-a]pyrimidin-4-one (8 mg, 91.2%). MS: m/z found 713.2 [M+H]⁺. ¹H NMR (400 MHz, Methanol-d₄): δ 9.12 (dd, J=7.5, 0.8 Hz, 1H), 8.41 (s, 1H), 7.77 (dd, J=2.0, 0.8 Hz, 1H), 7.73 (dd, J=7.5, 1.2 Hz, 1H), 7.65 (dd, J=7.7, 1.6 Hz, 1H), 7.63-7.56 (m, 2H), 7.55-7.48 (m, 3H), 7.39 (dd, J=7.6, 1.7 Hz, 1H), 7.24 (d, J=7.4 Hz, 1H), 4.09-4.03 (m, 1H), 4.01 (s, 3H), 3.91 (s, 2H), 3.88-3.79 (m, 4H), 3.78-3.71 (m, 1H), 2.81-2.62 (m, 4H), 2.36-2.22 (m, 3H), 2.09-1.98 (m, 1H), 1.94-1.86 (m, 2H), 1.86-1.76 (m, 1H), 1.63-1.49 (m, 1H).

### Example 319: 8-[2-Chloro-3-[2-chloro-3-[6-methoxy-5-[[[(2S)-5-oxopyrrolidin-2-yl]methylamino]methyl]-2-pyridyl]phenyl]phenyl]-3-[[[(2S)-2-hydroxypropyl]-methyl-amino]methyl]pyrido[1,2-a]pyrimidin-4-one

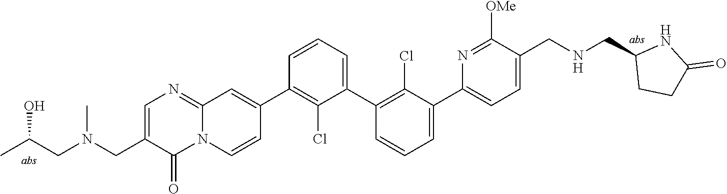 [SMILES: COc1nc(-c2cccc(-c3cccc(-c4ccn5c(=O)c(CN(C)C[C@H](C)O)cnc5c4)c3Cl)c2Cl)ccc1CNC[C@@H]1CCC(=O)N1]

### tert-Butyl N-[[6-[2-chloro-3-[2-chloro-3-[3-[[[(2S)-2-hydroxypropyl]-methyl-amino]methyl]-4-oxo-pyrido[1,2-a]pyrimidin-8-yl]phenyl]phenyl]-2-methoxy-3-pyridyl]methyl]-N-[[(2S)-5-oxopyrrolidin-2-yl]methyl]carbamate

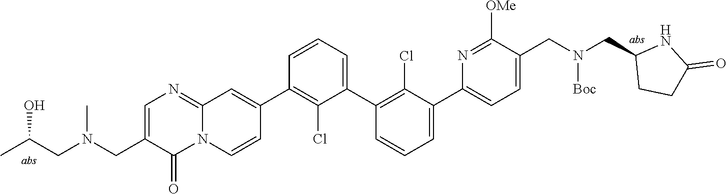 [SMILES: COc1nc(-c2cccc(-c3cccc(-c4ccn5c(=O)c(CN(C)C[C@H](C)O)cnc5c4)c3Cl)c2Cl)ccc1CN(C[C@@H]1CCC(=O)N1)C(=O)OC(C)(C)C]

tert-Butyl N-[[6-[2-chloro-3-[2-chloro-3-[3-[[[(2S)-2-hydroxypropyl]amino]methyl]-4-oxo-pyrido[1,2-a]pyrimidin-8-yl]phenyl]phenyl]-2-methoxy-3-pyridyl]methyl]-N-[[(2S)-5-oxopyrrolidin-2-yl]methyl]carbamate (20. mg, 0.03 mmol), formaldehyde (3.8 uL, 4.12 mg, 0.05 mmol) and NaBH₃CN (3.2 mg, 0.05 mmol) in MeOH/THF (2:1) was stirred at room temperature for 1 hour and directly purified by reversed phase chromatography to give tert-butyl N-[[6-[2-chloro-3-[2-chloro-3-[3-[[[(2S)-2-hydroxypropyl]-methyl-amino]methyl]-4-oxo-pyrido[1,2-a]pyrimidin-8-yl]phenyl]phenyl]-2-methoxy-3-pyridyl]methyl]-N-[[(2S)-5-oxopyrrolidin-2-yl]methyl]carbamate (18 mg, 88.4%). MS: m/z found 801.3 [M+H]⁺.

### 8-[2-Chloro-3-[2-chloro-3-[6-methoxy-5-[[[(2S)-5-oxopyrrolidin-2-yl]methylamino]methyl]-2-pyridyl]phenyl]phenyl]-3-[[[(2S)-2-hydroxypropyl]-methyl-amino]methyl]pyrido[1,2-a]pyrimidin-4-one

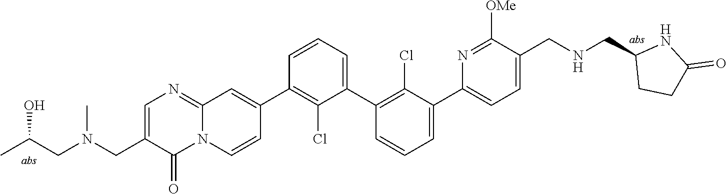 [SMILES: COc1nc(-c2cccc(-c3cccc(-c4ccn5c(=O)c(CN(C)C[C@H](C)O)cnc5c4)c3Cl)c2Cl)ccc1CNC[C@@H]1CCC(=O)N1]

A solution of tert-butyl N-[[6-[2-chloro-3-[2-chloro-3-[3-[[[(2S)-2-hydroxypropyl]-methyl-amino]methyl]-4-oxo-pyrido[1,2-a]pyrimidin-8-yl]phenyl]phenyl]-2-methoxy-3-pyridyl]methyl]-N-[[(2S)-5-oxopyrrolidin-2-yl]methyl]carbamate (10 mg, 0.01 mmol) in DCM/TFA (2:1) was stirred for 30 minutes, concentrated and purified by reversed phase chromatography to give 8-[2-chloro-3-[2-chloro-3-[6-methoxy-5-[[[(2S)-5-oxopyrrolidin-2-yl]methylamino]methyl]-2-pyridyl]phenyl]phenyl]-3-[[[(2S)-2-hydroxypropyl]-methyl-amino]methyl]pyrido[1,2-a]pyrimidin-4-one (8 mg, 91.4%). MS: m/z found 701.2 [M+H]⁺. ¹H NMR (400 MHz, Methanol-d₄): δ 9.16 (dd, J=7.4, 0.8 Hz, 1H), 8.46 (s, 1H), 7.81 (dd, J=2.0, 0.8 Hz, 1H), 7.74 (d, J=7.5 Hz, 1H), 7.66 (dd, J=7.7, 1.7 Hz, 1H), 7.64-7.55 (m, 3H), 7.54-7.48 (m, 2H), 7.40 (dd, J=7.6, 1.7 Hz, 1H), 7.25 (d, J=7.4 Hz, 1H), 4.09 (q, J=6.4 Hz, 1H), 4.02 (s, 3H), 3.98 (d, J=13.6 Hz, 1H), 3.90-3.80 (m, 4H), 2.80-2.64 (m, 4H), 2.52 (s, 3H), 2.38-2.24 (m, 3H), 1.86-1.76 (m, 1H), 1.18 (d, J=6.2 Hz, 3H).

### Example 320: Ethyl (3S)-4-[[8-[2-chloro-3-[2-chloro-3-[6-methoxy-5-[[[(2S)-5-oxopyrrolidin-2-yl]methylamino]methyl]-2-pyridyl]phenyl]phenyl]-4-oxo-pyrido[1,2-a]pyrimidin-3-yl]methylamino]-3-hydroxy-butanoate

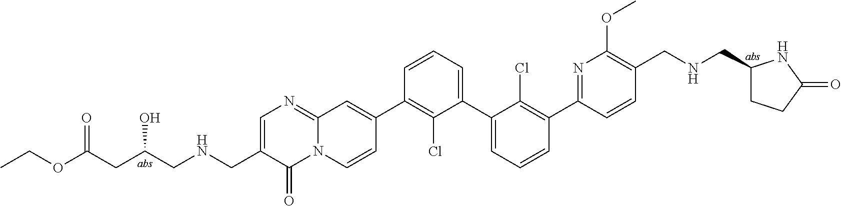 [SMILES: CCOC(=O)C[C@H](O)CNCc1cnc2cc(-c3cccc(-c4cccc(-c5ccc(CNC[C@@H]6CCC(=O)N6)c(OC)n5)c4Cl)c3Cl)ccn2c1=O]

To a solution of thionyl chloride (0.95 uL, 1.6 mg, 0.01 mmol) in EtOH at 0° C. was added (3S)-4-[[8-[2-chloro-3-[2-chloro-3-[6-methoxy-5-[[[(2S)-5-oxopyrrolidin-2-yl]methylamino]methyl]-2-pyridyl]phenyl]phenyl]-4-oxo-pyrido[1,2-a]pyrimidin-3-yl]methylamino]-3-hydroxy-butanoic acid (8 mg, 0.01 mmol). The mixture was stirred at 70° C. for 2 hours and concentrated. The purification by reversed phase chromatography provided ethyl (3S)-4-[[8-[2-chloro-3-[2-chloro-3-[6-methoxy-5-[[[(2S)-5-oxopyrrolidin-2-yl]methylamino]methyl]-2-pyridyl]phenyl]phenyl]-4-oxo-pyrido[1,2-a]pyrimidin-3-yl]methylamino]-3-hydroxy-butanoate (3 mg, 36.1%). MS: m/z found 759.2 [M+H]⁺. ¹H NMR (400 MHz, Methanol-d₄): δ 9.14 (d, J=7.4 Hz, 1H), 8.43 (s, 1H), 7.79 (d, J=1.1 Hz, 1H), 7.73 (dd, J=7.4, 0.8 Hz, 1H), 7.67-7.64 (m, 1H), 7.64-7.48 (m, 5H), 7.40 (ddd, J=7.6, 1.8, 0.9 Hz, 1H), 7.24 (dd, J=7.4, 0.9 Hz, 1H), 4.25-4.17 (m, 1H), 4.13 (q, J=7.1 Hz, 2H), 4.01 (s, 3H), 4.00-3.78 (m, 5H), 2.88-2.82 (m, 1H), 2.78-2.63 (m, 3H), 2.58-2.41 (m, 2H), 2.37-2.24 (m, 3H), 1.86-1.76 (m, 1H), 1.24 (t, J=7.1 Hz, 3H).

### Example 321: 8-[2-Chloro-3-[2-chloro-3-[6-methoxy-5-[[2-(2-oxopyrrolidin-1-yl)ethylamino]methyl]-2-pyridyl]phenyl]phenyl]-3-[[2-(2-oxopyrrolidin-1-yl)ethylamino]methyl]pyrido[1,2-a]pyrimidin-4-one

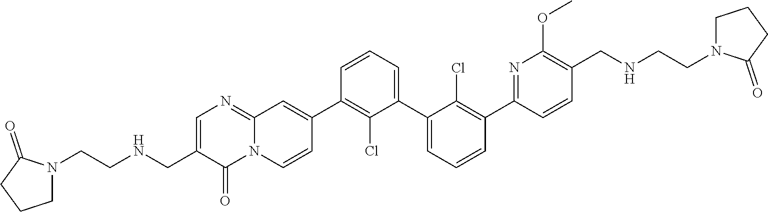 [SMILES: COc1nc(-c2cccc(-c3cccc(-c4ccn5c(=O)c(CNCCN6CCCC6=O)cnc5c4)c3Cl)c2Cl)ccc1CNCCN1CCCC1=O]

A mixture of 8-[2-chloro-3-[2-chloro-3-(5-formyl-6-methoxy-2-pyridyl)phenyl]phenyl]-4-oxo-pyrido[1,2-a]pyrimidine-3-carbaldehyde (15 mg, 0.03 mmol), 1-(2-aminoethyl)pyrrolidin-2-one (15 mg, 0.11 mmol) and acetic acid (3.2 uL, 3.4 mg, 0.06 mmol) in MeOH/THF (1:1) was stirred for 1.5 hour at rt. NaBH₃CN (7 mg, 0.11 mmol) was added, the mixture was stirred for additional 1 h, quenched with water and directly purified by reversed phase chromatography to give 8-[2-chloro-3-[2-chloro-3-[6-methoxy-5-[[2-(2-oxopyrrolidin-1-yl)ethylamino]methyl]-2-pyridyl]phenyl]phenyl]-3-[[2-(2-oxopyrrolidin-1-yl)ethylamino]methyl]pyrido[1,2-a]pyrimidin-4-one (17 mg, 79.6%). MS: m/z found 754.3 [M+H]⁺. ¹H NMR (400 MHz, Methanol-d₄): δ 9.17 (dd, J=7.4, 0.7 Hz, 1H), 8.49 (s, 1H), 7.88-7.80 (m, 2H), 7.67 (dd, J=7.7, 1.8 Hz, 1H), 7.66-7.59 (m, 3H), 7.56-7.50 (m, 2H), 7.43 (dd, J=7.6, 1.7 Hz, 1H), 7.33 (d, J=7.5 Hz, 1H), 4.22 (d, J=11.3 Hz, 4H), 4.07 (s, 3H), 3.62 (td, J=6.0, 1.4 Hz, 4H), 3.52 (td, J=7.1, 3.6 Hz, 4H), 3.21 (q, J=6.1 Hz, 4H), 2.42 (ddd, J=8.7, 7.6, 1.8 Hz, 4H), 2.15-2.04 (m, 4H).

### Example 322: 3-[[(1-Acetyl-4-piperidyl)amino]methyl]-8-[3-[3-[5-[[(1-acetyl-4-piperidyl)amino]methyl]-6-methoxy-2-pyridyl]-2-chloro-phenyl]-2-chloro-phenyl]pyrido[1,2-a]pyrimidin-4-one

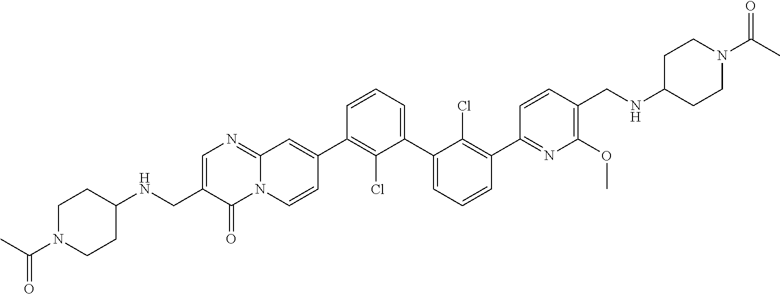 [SMILES: COc1nc(-c2cccc(-c3cccc(-c4ccn5c(=O)c(CNC6CCN(C(C)=O)CC6)cnc5c4)c3Cl)c2Cl)ccc1CNC1CCN(C(C)=O)CC1]

A mixture of 8-[2-chloro-3-[2-chloro-3-(5-formyl-6-methoxy-2-pyridyl)phenyl]phenyl]-4-oxo-pyrido[1,2-a]pyrimidine-3-carbaldehyde (15 mg, 0.03 mmol), 1-(4-amino-1-piperidyl)ethanone (16 mg, 0.11 mmol and acetic acid (3.2 uL, 3.4 mg, 0.06 mmol) in MeOH/THF (1:1) was stirred for 1.5 hour at rt. NaBH₃CN (7 mg, 0.11 mmol) was added, the mixture was stirred for additional 1 h, quenched with water and directly purified by reversed phase chromatography to give 3-[[(1-acetyl-4-piperidyl)amino]methyl]-8-[3-[3-[5-[[(1-acetyl-4-piperidyl)amino]methyl]-6-methoxy-2-pyridyl]-2-chloro-phenyl]-2-chloro-phenyl]pyrido[1,2-a]pyrimidin-4-one (18 mg, 81.3%). MS: m/z found 782.3 [M+H]⁺. ¹H NMR (400 MHz, Methanol-d₄): δ 9.17 (dd, J=7.4, 0.7 Hz, 1H), 8.50 (s, 1H), 7.87-7.80 (m, 2H), 7.70-7.58 (m, 4H), 7.56-7.50 (m, 2H), 7.43 (dd, J=7.6, 1.8 Hz, 1H), 7.32 (d, J=7.5 Hz, 1H), 4.65-4.52 (m, 2H), 4.15 (d, J=8.1 Hz, 4H), 4.06 (s, 3H), 4.05-3.98 (m, 2H), 3.27-3.14 (m, 4H), 2.70 (t, J=12.9 Hz, 2H), 2.25-2.14 (m, 4H), 1.63-1.36 (m, 4H).

### Example 335: 8-[2-Chloro-3-[2-chloro-3-[4-chloro-6-methoxy-5-[[[(2S)-5-oxopyrrolidin-2-yl]methylamino]methyl]-2-pyridyl]phenyl]phenyl]-3-[[[(2S)-5-oxopyrrolidin-2-yl]methylamino]methyl]pyrido[1,2-a]pyrimidin-4-one

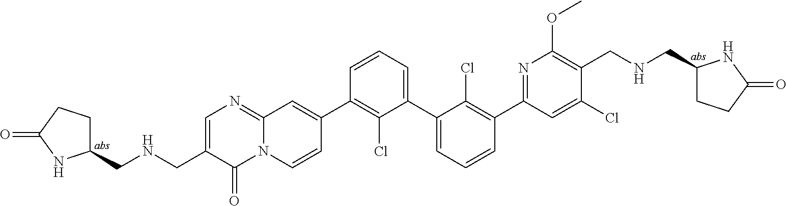 [SMILES: COc1nc(-c2cccc(-c3cccc(-c4ccn5c(=O)c(CNC[C@@H]6CCC(=O)N6)cnc5c4)c3Cl)c2Cl)cc(Cl)c1CNC[C@@H]1CCC(=O)N1]

### 4,6-Dichloro-2-methoxy-pyridine-3-carbaldehyde

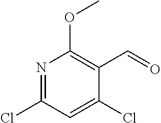

To a solution of 2,4-dichloro-6-methoxy-pyridine (0.50 g, 2.81 mmol) in dry THE (10 mL) at −78° C., n-butyllithium (1.6 M in hexanes, 1.1 eq.) was added dropwise. The reaction solution was stirred at −78° C. for 1 hour, followed by the addition of anhydrous N,N-dimethylformamide (0.33 mL, 0.31 g, 4.21 mmol) and the resulting mixture was stirred at −78° C. for another 1.5 hours. The reaction was quenched with glacial acetic acid (0.16 mL, 0.17 g, 2.81 mmol) and allowed to warm to room temperature. It was diluted with ethyl acetate, washed successively with saturated aqueous sodium bicarbonate and brine. The organic phase was dried over anhydrous sodium sulfate and evaporated under reduced pressure. The residue was purified by silica gel column chromatography (EtOAc in hexanes, 0% to 10% gradient) to give a mixture of 4,6-dichloro-2-methoxy-pyridine-3-carbaldehyde (0.30 g, 51.8%) and its regioisomer. ¹H NMR (400 MHz, Chloroform-d) δ 10.39 (s, 1H), 6.80 (s, 1H), 4.02 (s, 3H). (Regioisomer: ¹H NMR (400 MHz, Chloroform-d) δ 10.39 (s, 1H), 7.05 (s, 1H), 4.08 (s, 3H)).

### 8-[2-Chloro-3-[2-chloro-3-(4-chloro-5-formyl-6-methoxy-2-pyridyl)phenyl]phenyl]-4-oxo-pyrido[1,2-a]pyrimidine-3-carbaldehyde

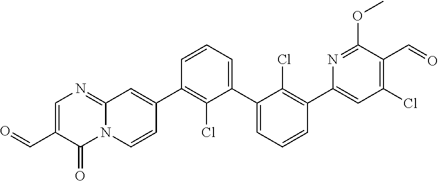 [SMILES: COc1nc(-c2cccc(-c3cccc(-c4ccn5c(=O)c(C=O)cnc5c4)c3Cl)c2Cl)cc(Cl)c1C=O]

To a solution of 8-[2-chloro-3-[2-chloro-3-(4,4,5,5-tetramethyl-1,3,2-dioxaborolan-2-yl)phenyl]phenyl]-4-oxo-pyrido[1,2-a]pyrimidine-3-carbaldehyde (111 mg, 0.21 mmol), 4,6-dichloro-2-methoxy-pyridine-3-carbaldehyde (44 mg, 0.21 mmol, a mixture of regioisomers) and potassium carbonate (89 mg, 0.64 mmol) in degassed dioxane/water (6:1) was added Pd(PPh₃)₄ (24.68 mg, 0.02 mmol) and the mixture was stirred at 90° C. for 2 hours under N₂(g). Water was added and the mixture was extracted into EtOAc three times. The combined organic layers were dried over anhydrous sodium sulfate, concentrated, and purified on silica gel column using EtOAc in hexanes (0 to 100% gradient) as eluent to give a mixture of regioisomer products, 60 mg total, 50%. The regioisomeric products (a ratio of ˜2:1) were separated by preparative HPLC. Major product is the desired product: MS: m/z found 564.0 [M+H]⁺. ¹H NMR (400 MHz, Chloroform-d): δ 10.52 (d, J=0.4 Hz, 1H), 10.41 (s, 1H), 9.32 (dd, J=7.3, 0.7 Hz, 1H), 8.95 (s, 1H), 7.95 (dd, J=1.9, 0.7 Hz, 1H), 7.70 (dd, J=7.7, 1.8 Hz, 1H), 7.62 (dd, J=7.2, 1.9 Hz, 1H), 7.58-7.48 (m, 4H), 7.44-7.40 (m, 2H), 4.13 (s, 3H). Minor undesired regioisomer: MS: m/z found 564.0 [M+H]⁺. ¹H NMR (400 MHz, Chloroform-d): δ 10.41 (s, 1H), 10.15-10.00 (m, 1H), 9.30 (dd, J=7.3, 0.7 Hz, 1H), 8.94 (s, 1H), 7.96-7.91 (m, 1H), 7.65-7.59 (m, 1H), 7.55-7.47 (m, 4H), 7.44-7.39 (m, 2H), 6.92 (s, 1H), 4.02 (s, 3H).

### 8-[2-Chloro-3-[2-chloro-3-[4-chloro-6-methoxy-5-[[[(2S)-5-oxopyrrolidin-2-yl]methylamino]methyl]-2-pyridyl]phenyl]phenyl]-3-[[[(2S)-5-oxopyrrolidin-2-yl]methylamino]methyl]pyrido[1,2-a]pyrimidin-4-one

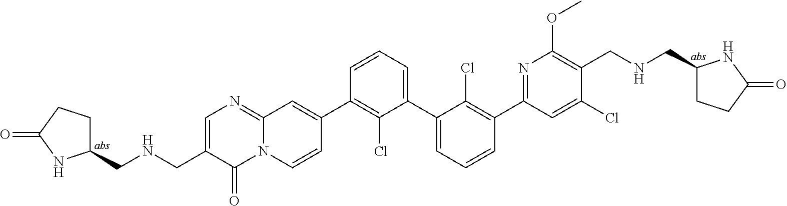 [SMILES: COc1nc(-c2cccc(-c3cccc(-c4ccn5c(=O)c(CNC[C@@H]6CCC(=O)N6)cnc5c4)c3Cl)c2Cl)cc(Cl)c1CNC[C@@H]1CCC(=O)N1]

A mixture of 8-[2-chloro-3-[2-chloro-3-(4-chloro-5-formyl-6-methoxy-2-pyridyl)phenyl]phenyl]-4-oxo-pyrido[1,2-a]pyrimidine-3-carbaldehyde (30 mg, 0.05 mmol), (5S)-5-(aminomethyl)pyrrolidin-2-one (24 mg, 0.21 mmol) and acetic acid (6.08 uL, 6.38 mg, 0.11 mmol) in MeOH/THF (1:1) was stirred for 1.5 hour at rt. NaBH₃CN (13.35 mg, 0.21 mmol) was added, the mixture was stirred for additional 1 h, quenched with water and directly purified by reversed phase chromatography to give 8-[2-chloro-3-[2-chloro-3-[4-chloro-6-methoxy-5-[[[(2S)-5-oxopyrrolidin-2-yl]methylamino]methyl]-2-pyridyl]phenyl]phenyl]-3-[[[(2S)-5-oxopyrrolidin-2-yl]methylamino]methyl]pyrido[1,2-a]pyrimidin-4-one (33 mg, 81.6%). MS: m/z found 760.2 [M+H]⁺. ¹H NMR (400 MHz, Methanol-d₄): δ 9.14 (dd, J=7.4, 0.8 Hz, 1H), 8.44 (s, 1H), 7.78 (dd, J=1.9, 0.8 Hz, 1H), 7.67 (dd, J=7.7, 1.7 Hz, 1H), 7.65-7.58 (m, 2H), 7.57-7.49 (m, 3H), 7.43 (dd, J=7.6, 1.7 Hz, 1H), 7.34 (s, 1H), 4.04 (s, 3H), 4.03 (s, 2H), 3.99-3.94 (m, 2H), 3.93-3.87 (m, 1H), 3.87-3.80 (m, 1H), 2.89-2.77 (m, 2H), 2.77-2.66 (m, 2H), 2.41-2.21 (m, 6H), 1.90-1.75 (m, 2H).

### Example 336: (3S)-4-[[8-[2-Chloro-3-[2-chloro-3-[4-fluoro-6-methoxy-5-[[[(2S)-5-oxopyrrolidin-2-yl]methylamino]methyl]-2-pyridyl]phenyl]phenyl]-4-oxo-pyrido[1,2-a]pyrimidin-3-yl]methylamino]-3-hydroxy-N,N-dimethyl-butanamide

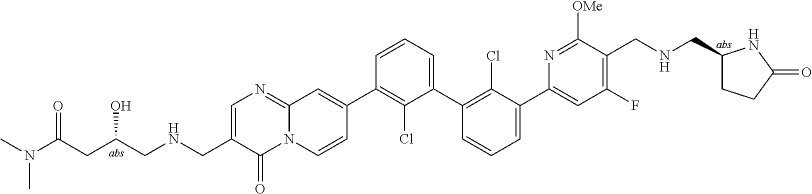 [SMILES: COc1nc(-c2cccc(-c3cccc(-c4ccn5c(=O)c(CNC[C@@H](O)CC(=O)N(C)C)cnc5c4)c3Cl)c2Cl)cc(F)c1CNC[C@@H]1CCC(=O)N1]

### tert-Butyl N-[[6-[2-chloro-3-[2-chloro-3-[3-[[[(2S)-4-(dimethylamino)-2-hydroxy-4-oxo-butyl]amino]methyl]-4-oxo-pyrido[1,2-a]pyrimidin-8-yl]phenyl]phenyl]-4-fluoro-2-methoxy-3-pyridyl]methyl]-N-[[(2S)-5-oxopyrrolidin-2-yl]methyl]carbamate

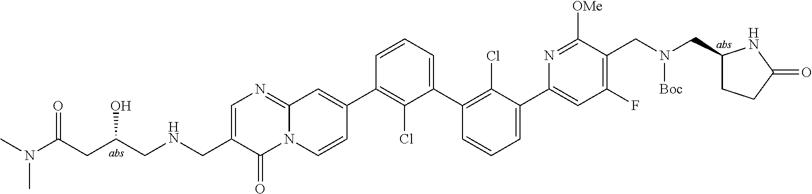 [SMILES: COc1nc(-c2cccc(-c3cccc(-c4ccn5c(=O)c(CNC[C@@H](O)CC(=O)N(C)C)cnc5c4)c3Cl)c2Cl)cc(F)c1CN(C[C@@H]1CCC(=O)N1)C(=O)OC(C)(C)C]

A mixture of tert-butyl N-[[6-[2-chloro-3-[2-chloro-3-(3-formyl-4-oxo-pyrido[1,2-a]pyrimidin-8-yl)phenyl]phenyl]-4-fluoro-2-methoxy-3-pyridyl]methyl]-N-[[(2S)-5-oxopyrrolidin-2-yl]methyl]carbamate (35 mg, 0.05 mmol), (3S)-4-amino-3-hydroxy-N,N-dimethyl-butanamide (14 mg, 0.09 mmol) and acetic acid (2.7 uL, 2.8 mg, 0.05 mmol) in MeOH/THF (2:1) was stirred for 1 hour at rt. NaBH₃CN (6 mg, 0.09 mmol) was added, the mixture was stirred for 1 hour, quenched with water, and purified by reversed phase chromatography to give tert-butyl N-[[6-[2-chloro-3-[2-chloro-3-[3-[[[(2S)-4-(dimethylamino)-2-hydroxy-4-oxo-butyl]amino]methyl]-4-oxo-pyrido[1,2-a]pyrimidin-8-yl]phenyl]phenyl]-4-fluoro-2-methoxy-3-pyridyl]methyl]-N-[[(2S)-5-oxopyrrolidin-2-yl]methyl]carbamate (32 mg, 77.9%). MS: m/z found 876.3 [M+H]⁺.

### (3S)-4-[[8-[2-Chloro-3-[2-chloro-3-[4-fluoro-6-methoxy-5-[[[(2S)-5-oxopyrrolidin-2-yl]methylamino]methyl]-2-pyridyl]phenyl]phenyl]-4-oxo-pyrido[1,2-a]pyrimidin-3-yl]methylamino]-3-hydroxy-N,N-dimethyl-butanamide

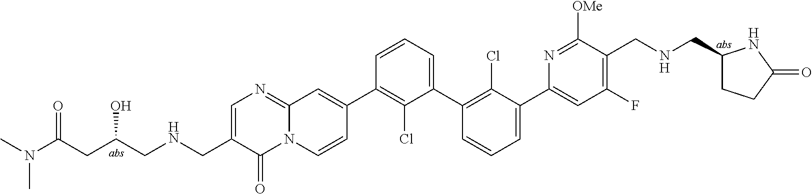 [SMILES: COc1nc(-c2cccc(-c3cccc(-c4ccn5c(=O)c(CNC[C@@H](O)CC(=O)N(C)C)cnc5c4)c3Cl)c2Cl)cc(F)c1CNC[C@@H]1CCC(=O)N1]

A solution of tert-butyl N-[[6-[2-chloro-3-[2-chloro-3-[3-[[[(2S)-4-(dimethylamino)-2-hydroxy-4-oxo-butyl]amino]methyl]-4-oxo-pyrido[1,2-a]pyrimidin-8-yl]phenyl]phenyl]-4-fluoro-2-methoxy-3-pyridyl]methyl]-N-[[(2S)-5-oxopyrrolidin-2-yl]methyl]carbamate (20 mg, 0.02 mmol) in DCM/TFA (2:1) was stirred for 30 minutes, concentrated and purified by reversed phase chromatography to give (3S)-4-[[8-[2-chloro-3-[2-chloro-3-[4-fluoro-6-methoxy-5-[[[(2S)-5-oxopyrrolidin-2-yl]methylamino]methyl]-2-pyridyl]phenyl]phenyl]-4-oxo-pyrido[1,2-a]pyrimidin-3-yl]methylamino]-3-hydroxy-N,N-dimethyl-butanamide (16 mg, 90.3%). MS: m/z found 776.3 [M+H]⁺. ¹H NMR (400 MHz, Methanol-d₄): δ 9.15 (dd, J=7.4, 0.7 Hz, 1H), 8.45 (s, 1H), 7.80 (dd, J=1.9, 0.7 Hz, 1H), 7.69 (dd, J=7.7, 1.7 Hz, 1H), 7.66-7.59 (m, 2H), 7.59-7.50 (m, 3H), 7.43 (dd, J=7.6, 1.7 Hz, 1H), 7.13 (d, J=9.2 Hz, 1H), 4.25 (tt, J=8.6, 4.6 Hz, 1H), 4.10-3.97 (m, 5H), 3.89 (s, 2H), 3.86-3.78 (m, 1H), 3.07 (s, 3H), 2.98-2.94 (m, 1H), 2.93 (s, 3H), 2.82 (dd, J=12.3, 8.6 Hz, 1H), 2.73-2.50 (m, 4H), 2.36-2.22 (m, 3H), 1.85-1.74 (m, 1H).

### Example 337: 8-[2-chloro-3-[2-chloro-3-[4-chloro-6-methoxy-3-[[[(2R)-5-oxopyrrolidin-2-yl]methylamino]methyl]-2-pyridyl]phenyl]phenyl]-3-[[[(2S)-5-oxopyrrolidin-2-yl]methylamino]methyl]pyrido[1,2-a]pyrimidin-4-one

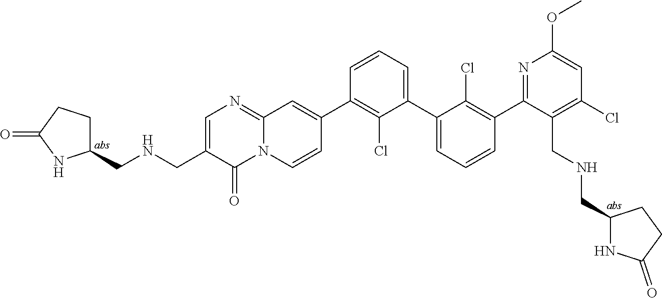 [SMILES: COc1cc(Cl)c(CNC[C@H]2CCC(=O)N2)c(-c2cccc(-c3cccc(-c4ccn5c(=O)c(CNC[C@@H]6CCC(=O)N6)cnc5c4)c3Cl)c2Cl)n1]

### 8-(2,2′-Dichloro-3′-(4-chloro-3-formyl-6-methoxypyridin-2-yl)-[1,1′-biphenyll-3-yl)-4-oxo-41-pyrido[1,2-a]pyrimidine-3-carbaldehyde

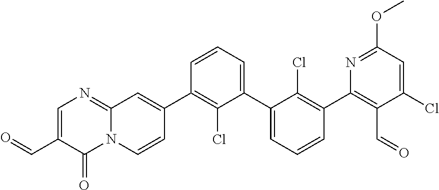 [SMILES: COc1cc(Cl)c(C=O)c(-c2cccc(-c3cccc(-c4ccn5c(=O)c(C=O)cnc5c4)c3Cl)c2Cl)n1]

To a solution of 8-[2-chloro-3-[2-chloro-3-(4,4,5,5-tetramethyl-1,3,2-dioxaborolan-2-yl)phenyl]phenyl]-4-oxo-pyrido[1,2-a]pyrimidine-3-carbaldehyde (111 mg, 0.21 mmol), 2,4-dichloro-6-methoxynicotinaldehyde (44 mg, 0.21 mmol, a mixture of regioisomers) and potassium carbonate (89 mg, 0.64 mmol) in degassed dioxane/water (6:1) was added Pd(PPh₃)₄ (24.68 mg, 0.02 mmol) and the mixture was stirred at 90° C. for 2 hours under N₂(g). Water was added and the mixture was extracted into EtOAc three times. The combined organic layers were dried over anhydrous sodium sulfate, concentrated, and purified on silica gel column using EtOAc in hexanes (0 to 100% gradient) as eluent to give a mixture of regioisomer products, 60 mg total, 50%. The regioisomers (a ratio of −2:1) were separated by preparative HPLC. Minor product is the desired product. MS: m/z found 564.0 [M+H]⁺. ¹H NMR (400 MHz, Chloroform-d): δ 10.41 (s, 1H), 10.15-10.00 (m, 1H), 9.30 (dd, J=7.3, 0.7 Hz, 1H), 8.94 (s, 1H), 7.96-7.91 (m, 1H), 7.65-7.59 (m, 1H), 7.55-7.47 (m, 4H), 7.44-7.39 (m, 2H), 6.92 (s, 1H), 4.02 (s, 3H).

### 8-[2-Chloro-3-[2-chloro-3-[4-chloro-6-methoxy-3-[[[(2R)-5-oxopyrrolidin-2-yl]methylamino]methyl]-2-pyridyl]phenyl]phenyl]-3-[[[(2S)-5-oxopyrrolidin-2-yl]methylamino]methyl]pyrido[1,2-a]pyrimidin-4-one

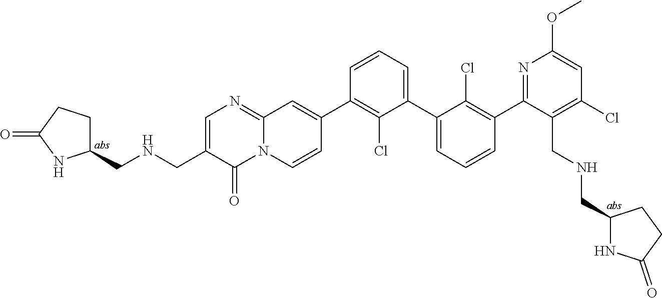 [SMILES: COc1cc(Cl)c(CNC[C@H]2CCC(=O)N2)c(-c2cccc(-c3cccc(-c4ccn5c(=O)c(CNC[C@@H]6CCC(=O)N6)cnc5c4)c3Cl)c2Cl)n1]

A mixture of 8-[2-chloro-3-[2-chloro-3-(4-chloro-3-formyl-6-methoxy-2-pyridyl)phenyl]phenyl]-4-oxo-pyrido[1,2-a]pyrimidine-3-carbaldehyde (24. mg, 0.04 mmol), (5S)-5-(aminomethyl)pyrrolidin-2-one (19 mg, 0.17 mmol) and acetic acid (5 uL, 5 mg, 0.08 mmol) in MeOH/THF (1:1) was stirred for 1.5 hour at rt. NaBH₃CN (11 mg, 0.17 mmol) was added, the mixture was stirred for additional 1 h, quenched with water and directly purified by reversed phase chromatography to give 8-[2-chloro-3-[2-chloro-3-[4-chloro-6-methoxy-3-[[[(2R)-5-oxopyrrolidin-2-yl]methylamino]methyl]-2-pyridyl]phenyl]phenyl]-3-[[[(2S)-5-oxopyrrolidin-2-yl]methylamino]methyl]pyrido[1,2-a]pyrimidin-4-one (26 mg, 80.4%). MS: m/z found 760.2 [M+H]⁺. ¹H NMR (400 MHz, Methanol-d₄): δ 9.15 (dd, J=7.4, 3.4 Hz, 1H), 8.44 (s, 1H), 7.79 (d, J=1.8 Hz, 1H), 7.66-7.60 (m, 2H), 7.60-7.51 (m, 4H), 7.49 (dd, J=7.1, 2.1 Hz, 1H), 7.00 (s, 1H), 4.04-3.94 (m, 2H), 3.94-3.84 (m, 5H), 3.66-3.58 (m, 1H), 3.57-3.47 (m, 1H), 2.91-2.76 (m, 2H), 2.55-2.24 (m, 6H), 2.23-2.17 (m, 1H), 2.15-2.02 (m, 1H), 1.89-1.79 (m, 1H), 1.74-1.57 (m, 1H).

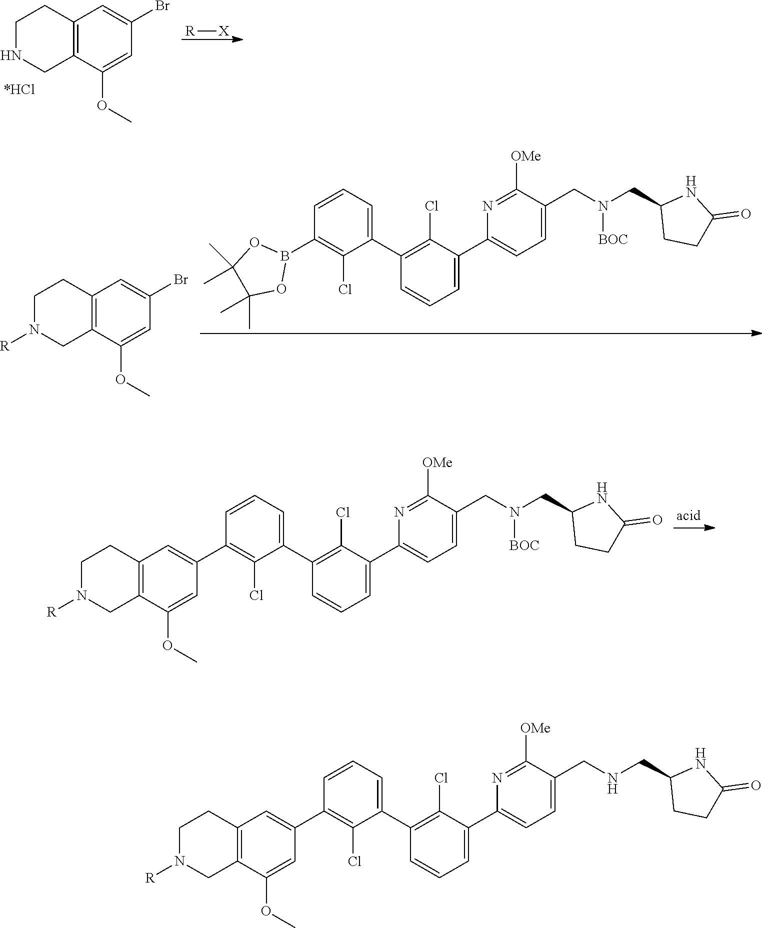

### Example 498: (S)-5-((((6-(3′-(2-((1-acetylpiperidin-4-yl)methyl)-8-methoxy-1,2,3,4-tetrahydroisoquinolin-6-yl)-2,2′-dichloro-[1,1′-biphenyl]-3-yl)-2-methoxypyridin-3-yl)methyl)amino)methyl)pyrrolidin-2-one

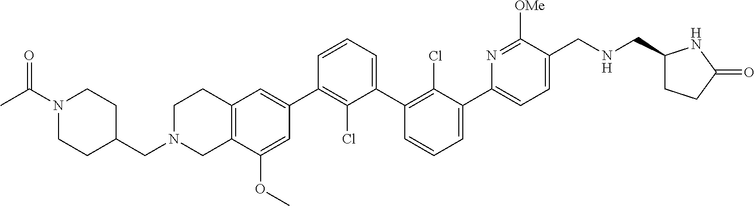 [SMILES: COc1cc(-c2cccc(-c3cccc(-c4ccc(CNC[C@@H]5CCC(=O)N5)c(OC)n4)c3Cl)c2Cl)cc2c1CN(CC1CCN(C(C)=O)CC1)CC2]

### 1-(4-((6-Bromo-8-methoxy-3,4-dihydroisoquinolin-2(1H)-yl)methyl)piperidin-1-yl)ethan-1-one

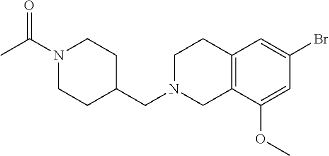 [SMILES: COc1cc(Br)cc2c1CN(CC1CCN(C(C)=O)CC1)CC2]

A scintillation vial was charged with 6-bromo-8-methoxy-1,2,3,4-tetrahydroisoquinoline hydrochloride (0.15 g, 0.54 mmol), 1-(4-(bromomethyl)piperidin-1-yl)ethan-1-one (0.24 g, 1.08 mmol), and K₂CO₃ (0.22 g, 1.62 mmol). ACN was then added (5 ml), and the reaction was stirred at 80° C., for 2 hr. The mixture was concentrated under reduced pressure. The residue was purified by normal phase flash SiO₂ chromatography using a gradient (0-10% methanol DCM) to afford 1-(4-((6-Bromo-8-methoxy-3,4-dihydroisoquinolin-2(1H)-yl)methyl)piperidin-1-yl)ethan-1-one (0.1 g, 100% yield). MS: m/z found 381 [M+H]⁺.

### tert-Butyl (S)-((6-(3′-(2-((1-acetylpiperidin-4-yl)methyl)-8-methoxy-1,2,3,4-tetrahydroisoquinolin-6-yl)-2,2′-dichloro-[1,1′-biphenyl]-3-yl)-2-methoxypyridin-3-yl)methyl)((5-oxopyrrolidin-2-yl)methyl)carbamate

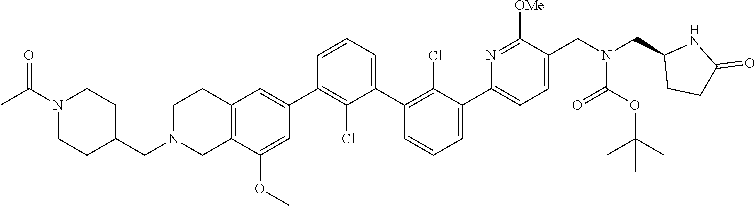 [SMILES: COc1cc(-c2cccc(-c3cccc(-c4ccc(CN(C[C@@H]5CCC(=O)N5)C(=O)OC(C)(C)C)c(OC)n4)c3Cl)c2Cl)cc2c1CN(CC1CCN(C(C)=O)CC1)CC2]

A microwave vial was charged with 1-(4-((6-bromo-8-methoxy-3,4-dihydroisoquinolin-2(1H)-yl)methyl)piperidin-1-yl)ethan-1-one (0.06 g, 0.16 mmol), tert-butyl (S)-((6-(2,2′-dichloro-3′-(4,4,5,5-tetramethyl-1,3,2-dioxaborolan-2-yl)-[1,1′-biphenyl]-3-yl)-2-methoxypyridin-3-yl)methyl)((5-oxopyrrolidin-2-yl)methyl)carbamate (0.11 g, 0.16 mmol), Cs₂CO₃ (0.15 g 0.47 mmol), and Pd(PPh₃)₄ (0.02 g, 0.02 mmol). Dioxane was added (2 ml), and the reaction was sparged with nitrogen, then 2 ml of water added. The mixture was stirred at 90° C. for 1 hr thermally. The mixture was filtered through CELITE® and the filter cake was washed with EtOAc (3×15 mL). The filtrate was concentrated under reduced pressure. The residue was purified by normal phase flash SiO₂ chromatography using a gradient (0-5% methanol DCM) to afford tert-butyl (S)-((6-(3′-(2-((1-acetylpiperidin-4-yl)methyl)-8-methoxy-1,2,3,4-tetrahydroisoquinolin-6-yl)-2,2′-dichloro-[1,1′-biphenyl]-3-yl)-2-methoxypyridin-3-yl)methyl)((5-oxopyrrolidin-2-yl)methyl)carbamate (0.087 g, 64% yield). MS: m/z found 856 [M+H]⁺.

### (S)-5-((((6-(3′-(2-((1-Acetylpiperidin-4-yl)methyl)-8-methoxy-1,2,3,4-tetrahydroisoquinolin-6-yl)-2,2′-dichloro-[1,1′-biphenyl]-3-yl)-2-methoxypyridin-3-yl)methyl)amino)methyl)pyrrolidin-2-one

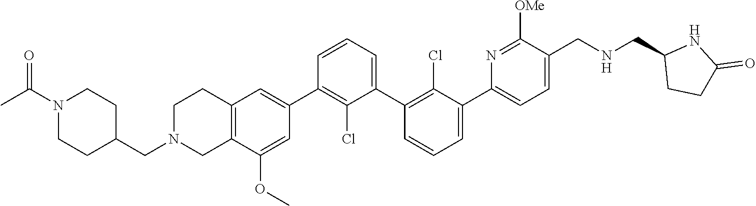 [SMILES: COc1cc(-c2cccc(-c3cccc(-c4ccc(CNC[C@@H]5CCC(=O)N5)c(OC)n4)c3Cl)c2Cl)cc2c1CN(CC1CCN(C(C)=O)CC1)CC2]

A scintillation vial was charged with tert-Butyl (S)-((6-(3′-(2-((1-acetylpiperidin-4-yl)methyl)-8-methoxy-1,2,3,4-tetrahydroisoquinolin-6-yl)-2,2′-dichloro-[1,1′-biphenyl]-3-yl)-2-methoxypyridin-3-yl)methyl)((5-oxopyrrolidin-2-yl)methyl)carbamate (0.09 g, 0.10 mmol), then 5 ml of a 1:1 solution of TFA/DCM was added, and the reaction was stirred at room temp for 10 min. The reaction was concentrated, and residue purified by Semi-prep HPLC (with 0.05% Formic acid modifier). ACN was evaporated off, and aqueous portion was lyophilized to give 34.5 mg (45%) of the product as bis formic acid salt affording (S)-5-((((6-(3′-(2-((1-acetylpiperidin-4-yl)methyl)-8-methoxy-1,2,3,4-tetrahydroisoquinolin-6-yl)-2,2′-dichloro-[1,1′-biphenyl]-3-yl)-2-methoxypyridin-3-yl)methyl)amino)methyl)pyrrolidin-2-one as a white solid. MS: m/z found 756 [M+H]⁺. ¹H NMR (400 MHz, Methanol-d₄) δ 8.44 (s, 1H), 7.73 (d, J=7.5 Hz, 1H), 7.59 (dd, J=7.7, 1.7 Hz, 1H), 7.49-7.20 (m, 6H), 6.82 (d, J=2.8 Hz, 2H), 4.51 (d, J=13.1 Hz, 1H), 4.00 (s, 3H), 3.97-3.83 (m, 3H), 3.81 (d, J=14.5 Hz, 6H), 3.14 (t, 1H), 2.97 (s, 4H), 2.70-2.59 (m, 5H), 2.39-2.20 (m, 3H), 2.07 (s, 4H), 1.93-1.73 (m, 3H), 1.28-1.04 (m, 2H).

### Example 497: (S)-5-((((6-(2,2′-Dichloro-3′-(8-methoxy-2-(2-(piperidin-4-yl)ethyl)-1,2,3,4-tetrahydroisoquinolin-6-yl)-[1,1′-biphenyl]-3-yl)-2-methoxypyridin-3-yl)methyl)amino)methyl)pyrrolidin-2-one

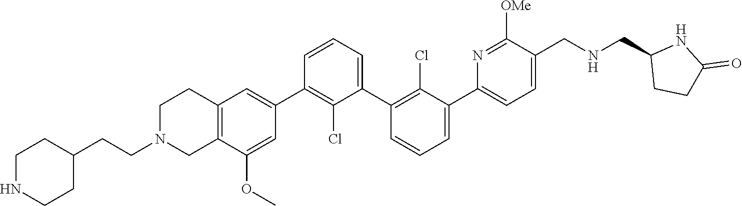 [SMILES: COc1cc(-c2cccc(-c3cccc(-c4ccc(CNC[C@@H]5CCC(=O)N5)c(OC)n4)c3Cl)c2Cl)cc2c1CN(CCC1CCNCC1)CC2]

This compound was prepared in a similar fashion to Example 498, using tert-butyl 4-(2-bromoethyl)piperidine-1-carboxylate and 6-bromo-8-methoxy-1,2,3,4-tetrahydroisoquinoline hydrochloride. MS: m/z found 728 [M+H]⁺; ¹H NMR (400 MHz, Methanol-d₄) δ 8.38 (s, 1H), 7.76 (d, J=7.5 Hz, 1H), 7.59 (dd, J=7.7, 1.7 Hz, 1H), 7.50-7.34 (m, 3H), 7.36-7.22 (m, 3H), 6.85 (d, J=3.8 Hz, 2H), 4.06-3.85 (m, 8H), 3.84 (s, 3H), 3.37 (d, J=12.9 Hz, 2H), 3.12 (s, 2H), 3.06 (d, J=5.7 Hz, 2H), 3.01-2.81 (m, 6H), 2.40-2.22 (m, 3H), 1.98 (d, J=14.1 Hz, 2H), 1.87-1.75 (m, 1H), 1.74-1.69 (m, 3H), 1.44 (t, J=11.7 Hz, 2H).

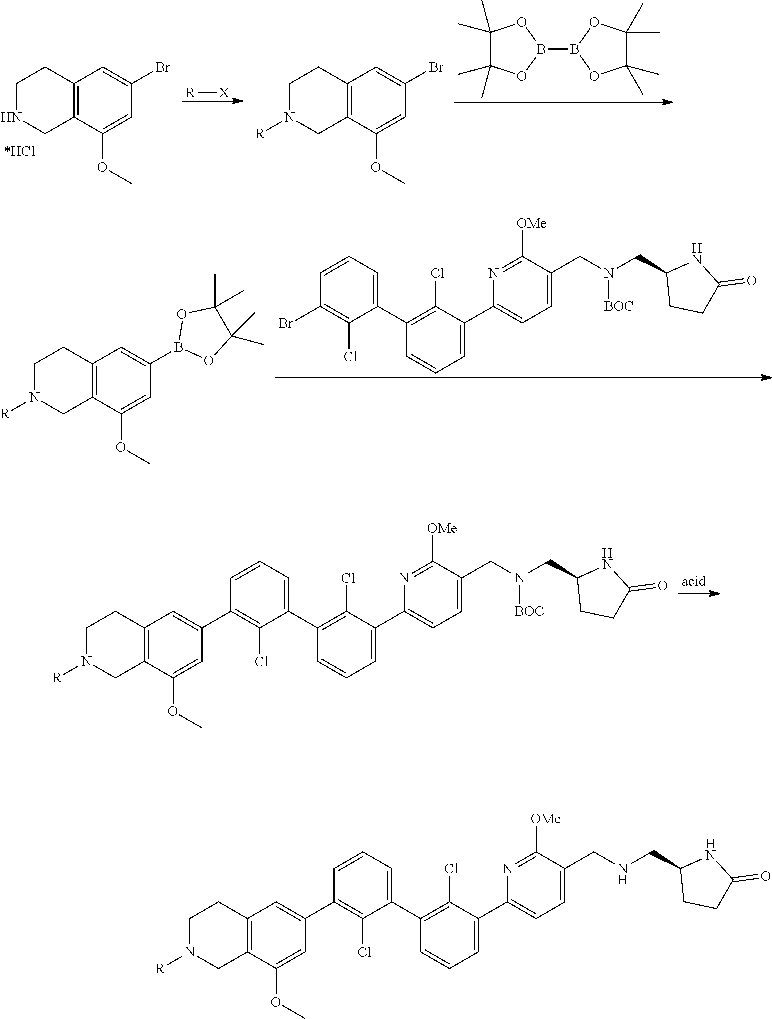

### Example 488: (5S)-5-((((6-(2,2′-Dichloro-3′-(2-(2-hydroxy-3-methylbutyl)-8-methoxy-1,2,3,4-tetrahydroisoquinolin-6-yl)-[1,1′-biphenyl]-3-yl)-2-methoxypyridin-3-yl)methyl)amino)methyl)pyrrolidin-2-one

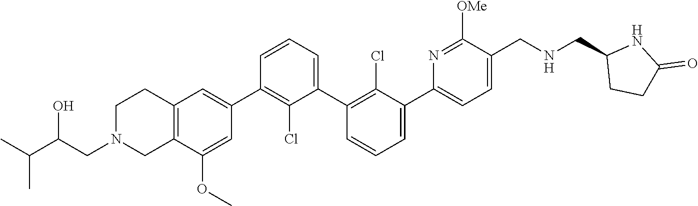 [SMILES: COc1cc(-c2cccc(-c3cccc(-c4ccc(CNC[C@@H]5CCC(=O)N5)c(OC)n4)c3Cl)c2Cl)cc2c1CN(CC(O)C(C)C)CC2]

### 1-(6-Bromo-8-methoxy-3,4-dihydroisoquinolin-2(1H)-yl)-3-methylbutan-2-ol

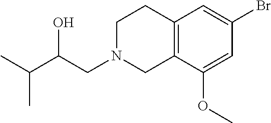 [SMILES: COc1cc(Br)cc2c1CN(CC(O)C(C)C)CC2]

A scintillation vial was charged with 6-bromo-8-methoxy-1,2,3,4-tetrahydroisoquinoline hydrochloride (0.10 g, 0.36 mmol), 1-bromo-3-methylbutan-2-ol (0.30 g, 1.79 mmol), and K₂CO₃ (0.15 g, 1.08 mmol). Acetonitrile was then added (5 ml), and the reaction was stirred at 80° C., for 2 hr. The mixture was concentrated under reduced pressure. The residue was purified by normal phase flash SiO₂ chromatography using a gradient (0-10% methanol DCM) to afford 1-(6-bromo-8-methoxy-3,4-dihydroisoquinolin-2(1H)-yl)-3-methylbutan-2-ol (0.120 g, 100% yield). MS: m/z found 328 [M+H]⁺. 1-(8-Methoxy-6-(4,4,5,5-tetramethyl-1,3,2-dioxaborolan-2-yl)-3,4-dihydroisoquinolin-2(1H)-yl)-3-methylbutan-2-ol

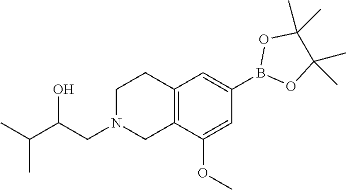 [SMILES: COc1cc(B2OC(C)(C)C(C)(C)O2)cc2c1CN(CC(O)C(C)C)CC2]

A microwave vial was charged with 1-(6-bromo-8-methoxy-3,4-dihydroisoquinolin-2(1H)-yl)-3-methylbutan-2-ol (0.15 g, 0.47 mmol), Bis(pinacolato)diboron (0.13 g, 0.51 mmol), KOAc (0.13 g, 1.31 mmol), and Pd(dppf)₂C12-DCM (0.04 g, 0.05 mmol). The flask was purged with nitrogen for 5 min, then 2 ml of dry dioxane was added, and the reaction was sparged with nitrogen for 5 min. The mixture was stirred at 90° C. for 1 hr thermally. The mixture was filtered through CELITE® and the filter cake was washed with EtOAc (3×40 mL). The filtrate was concentrated under reduced pressure. The residue was purified by normal phase flash SiO₂ chromatography using a gradient (0-10% methanol DCM) to afford 1-(8-Methoxy-6-(4,4,5,5-tetramethyl-1,3,2-dioxaborolan-2-yl)-3,4-dihydroisoquinolin-2(1H)-yl)-3-methylbutan-2-ol (0.175 g, 100% yield). MS: m/z found 376 [M+H]⁺.

### tert-Butyl ((6-(2,2′-dichloro-3′-(2-(2-hydroxy-3-methylbutyl)-8-methoxy-1,2,3,4-tetrahydroisoquinolin-6-yl)-[1,1′-biphenyl]-3-yl)-2-methoxypyridin-3-yl)methyl)(((S)-5-oxopyrrolidin-2-yl)methyl)carbamate

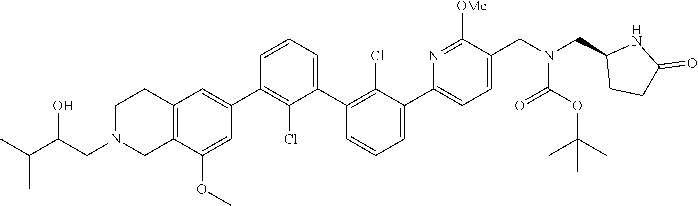 [SMILES: COc1cc(-c2cccc(-c3cccc(-c4ccc(CN(C[C@@H]5CCC(=O)N5)C(=O)OC(C)(C)C)c(OC)n4)c3Cl)c2Cl)cc2c1CN(CC(O)C(C)C)CC2]

A microwave vial was charged with 1-(8-Methoxy-6-(4,4,5,5-tetramethyl-1,3,2-dioxaborolan-2-yl)-3,4-dihydroisoquinolin-2(1H)-yl)-3-methylbutan-2-ol (0.06 g, 0.16 mmol), tert-butyl (S)-((6-(3′-bromo-2,2′-dichloro-[1,1′-biphenyl]-3-yl)-2-methoxypyridin-3-yl)methyl)((5-oxopyrrolidin-2-yl)methyl)carbamate (0.10 g, 0.16 mmol), Cs₂CO₃ (0.16 g 0.48 mmol), and Pd(PPh₃)₄ (0.02 g, 0.02 mmol). Dioxane was added (2 ml), and the reaction was sparged with nitrogen, then 2 ml of water added. The mixture was stirred at 90° C. for 1 hr. The mixture was filtered through CELITE® and the filter cake was washed with EtOAc (3×15 mL). The filtrate was concentrated under reduced pressure. The residue was purified by normal phase flash SiO₂ chromatography using a gradient (0-10% methanol DCM) to afford tert-Butyl ((6-(2,2′-dichloro-3′-(2-(2-hydroxy-3-methylbutyl)-8-methoxy-1,2,3,4-tetrahydroisoquinolin-6-yl)-[1,1′-biphenyl]-3-yl)-2-methoxypyridin-3-yl)methyl)(((S)-5-oxopyrrolidin-2-yl)methyl)carbamate (0.08 g, 70% yield). MS: m/z found 803 [M+H]⁺.

### (5S)-5-((((6-(2,2′-Dichloro-3′-(2-(2-hydroxy-3-methylbutyl)-8-methoxy-1,2,3,4-tetrahydroisoquinolin-6-yl)-[1,1′-biphenyl]-3-yl)-2-methoxypyridin-3-yl)methyl)amino)methyl)pyrrolidin-2-one

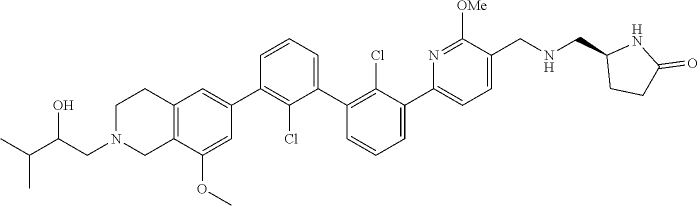 [SMILES: COc1cc(-c2cccc(-c3cccc(-c4ccc(CNC[C@@H]5CCC(=O)N5)c(OC)n4)c3Cl)c2Cl)cc2c1CN(CC(O)C(C)C)CC2]

A scintillation vial was charged with tert-Butyl ((6-(2,2′-dichloro-3′-(2-(2-hydroxy-3-methylbutyl)-8-methoxy-1,2,3,4-tetrahydroisoquinolin-6-yl)-[1,1′-biphenyl]-3-yl)-2-methoxypyridin-3-yl)methyl)(((S)-5-oxopyrrolidin-2-yl)methyl)carbamate (0.09 g, 0.11 mmol), then 5 ml of a 1:1 solution of TFA/DCM was added, and the reaction was stirred at room temp for 10 min. The reaction was concentrated, and residue purified by Semi-prep HPLC (with 0.05% Formic acid modifier). Acetonitrile was evaporated off, and aqueous portion was lyophilized to give 10.0 mg (13%) of the product as bis formic acid salt affording (5S)-5-((((6-(2,2′-Dichloro-3′-(2-(2-hydroxy-3-methylbutyl)-8-methoxy-1,2,3,4-tetrahydroisoquinolin-6-yl)-[1,1′-biphenyl]-3-yl)-2-methoxypyridin-3-yl)methyl)amino)methyl)pyrrolidin-2-one as a white solid. MS: m/z found 703 [M+H]⁺. ¹H NMR (400 MHz, Methanol-d₄) δ 8.49 (s, 1H), 7.70 (d, J=7.5 Hz, 1H), 7.59 (d, J=7.7, 1.7 Hz, 1H), 7.49-7.35 (m, 3H), 7.31 (t, J=7.3, 1.9 Hz, 2H), 7.21 (d, J=7.4 Hz, 1H), 6.86 (d, J=4.8 Hz, 2H), 4.09 (s, 1H), 3.99 (s, 3H), 3.83 (d, J=10.2 Hz, 7H), 3.72 (t, J=6.1 Hz, 1H), 3.24-3.19 (m, 1H), 3.11-2.86 (m, 5H), 2.76-2.62 (m, 2H), 2.36-2.20 (m, 3H), 1.81-1.76 (m, 1H), 1.74-1.64 (m, 1H), 0.96 (d, J=6.8, 5.2 Hz, 6H).

### Example 485: (S)-5-((((6-(2,2′-Dichloro-3′-(2-(3-hydroxy-3-methylbutyl)-8-methoxy-1,2,3,4-tetrahydroisoquinolin-6-yl)-[1,1′-biphenyl]-3-yl)-2-methoxypyridin-3-yl)methyl)amino)methyl)pyrrolidin-2-one

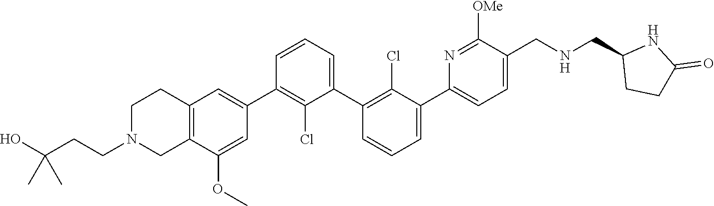 [SMILES: COc1cc(-c2cccc(-c3cccc(-c4ccc(CNC[C@@H]5CCC(=O)N5)c(OC)n4)c3Cl)c2Cl)cc2c1CN(CCC(C)(C)O)CC2]

This compound was prepared in a similar fashion to Example 488, using 4-bromo-2-methylbutan-2-ol and 6-bromo-8-methoxy-1,2,3,4-tetrahydroisoquinoline hydrochloride. MS: m/z found 703 [M+H]⁺; ¹H NMR (400 MHz, Methanol-d₄) δ 7.77 (d, J=7.5 Hz, 1H), 7.60 (dd, J=7.7, 1.7 Hz, 1H), 7.50-7.36 (m, 3H), 7.32 (t, J=7.6, 1.9 Hz, 2H), 7.26 (d, J=7.5 Hz, 1H), 6.91 (d, 2H), 4.26 (s, 2H), 4.02 (s, 5H), 3.90 (t, J=6.6 Hz, 1H), 3.87 (s, 3H), 3.46 (t, 2H), 3.39-3.30 (m, 2H), 3.15 (t, J=6.2 Hz, 2H), 2.97-2.83 (m, 2H), 2.41-2.23 (m, 3H), 1.93 (t, 2H), 1.90-1.76 (m, 1H), 1.27 (s, 6H).

### Example 484: (S)-5-((((6-(2,2′-Dichloro-3′-(2-(2-(1-hydroxycyclobutyl)ethyl)-8-methoxy-1,2,3,4-tetrahydroisoquinolin-6-yl)-[1,1′-biphenyl]-3-yl)-2-methoxypyridin-3-yl)methyl)amino)methyl)pyrrolidin-2-one

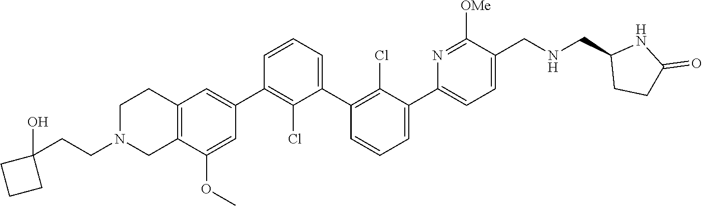 [SMILES: COc1cc(-c2cccc(-c3cccc(-c4ccc(CNC[C@@H]5CCC(=O)N5)c(OC)n4)c3Cl)c2Cl)cc2c1CN(CCC1(O)CCC1)CC2]

This compound was prepared in a similar fashion to Example 488, using 1-(2-bromoethyl)cyclobutan-1-ol and 6-bromo-8-methoxy-1,2,3,4-tetrahydroisoquinoline hydrochloride. MS: m/z found 715 [M+H]⁺; ¹H NMR (400 MHz, Methanol-d₄) δ 7.86 (d, J=7.6 Hz, 1H), 7.61 (dd, J=7.7, 1.7 Hz, 1H), 7.52-7.37 (m, 3H), 7.40-7.29 (m, 3H), 6.94 (d, 2H), 4.63 (s, 1H), 4.31 (s, 2H), 4.21-4.16 (m, 1H), 4.07 (s, 3H), 4.07-3.97 (m, 1H), 3.88 (s, 3H), 3.42 (s, 3H), 3.32-3.09 (m, 4H), 2.45-2.29 (m, 3H), 2.11 (t, J=8.9, 6.7 Hz, 7H), 1.97-1.83 (m, 1H), 1.81-1.68 (m, 1H), 1.67-1.53 (m, 1H).

### Example 424: (S)-5-((((6-(2,2′-Dichloro-3′-(5-methoxy-2-(((S)-5-oxopyrrolidin-2-yl)methyl)-1,2,3,4-tetrahydroisoquinolin-7-yl)-[1,1′-biphenyl]-3-yl)-2-methoxypyridin-3-yl)methyl)amino)methyl)pyrrolidin-2-one

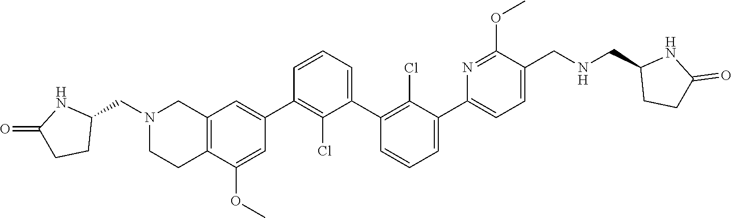 [SMILES: COc1cc(-c2cccc(-c3cccc(-c4ccc(CNC[C@@H]5CCC(=O)N5)c(OC)n4)c3Cl)c2Cl)cc2c1CCN(C[C@@H]1CCC(=O)N1)C2]

### (S)-5-((7-bromo-5-methoxy-3,4-diliydroisoquinolin-2(1H)-yl)methyl)pyrrolidin-2-one:

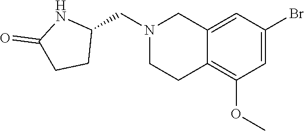 [SMILES: COc1cc(Br)cc2c1CCN(C[C@@H]1CCC(=O)N1)C2]

A mixture of 7-bromo-5-methoxy-1,2,3,4-tetrahydroisoquinoline hydrochloride (75 mg, 0.31 mmol), (2S)-5-oxopyrrolidin-2-yl]methyl methanesulfonate (60 mg, 0.31 mmol), and potassium carbonate (70 mg, 0.5 mmol) in 3 mL acetonitrile were stirred at 80° C. for 12 hours. Reaction was filtered through CELITE®. The filtrate was concentrated under reduced pressure and the crude oil was purified by normal phase SiO₂ chromatography (0-100% EtOAc/hexane) to afford (S)-5-((7-bromo-5-methoxy-3,4-dihydroisoquinolin-2(1H)-yl)methyl)pyrrolidin-2-one (85 mg, 80.7% yield) as a white solid. m/z: 340 [M+H]⁺ observed.

### (S)-5-((((6-(2,2′-Dichloro-3′-(5-methoxy-2-(((S)-5-oxopyrrolidin-2-yl)methyl)-1,2,3,4-tetrahydroisoquinolin-7-yl)-[1,1′-biphenyl]-3-yl)-2-methoxypyridin-3-yl)methyl)amino)methyl)pyrrolidin-2-one

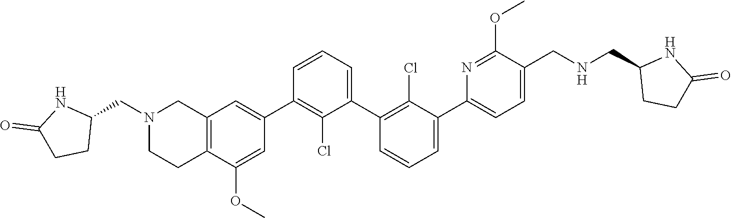 [SMILES: COc1cc(-c2cccc(-c3cccc(-c4ccc(CNC[C@@H]5CCC(=O)N5)c(OC)n4)c3Cl)c2Cl)cc2c1CCN(C[C@@H]1CCC(=O)N1)C2]

A mixture of(5S)-5-[(2-chloro-7,8-dihydro-5H-1,6-naphthyridin-6-yl)methyl]pyrrolidin-2-one (65 mg, 0.20 mmol), tert-butyl N-[[6-[2-chloro-3-[2-chloro-3-(4,4,5,5-tetramethyl-1,3,2-dioxaborolan-2-yl)phenyl]phenyl]-2-methoxy-3-pyridyl]methyl]-N-[[(2S)-5-oxopyrrolidin-2-yl]methyl]carbamate (140 mg, 0.21 mmol), 1,1′-bis(diphenylphosphino)-ferrocene-palladium(ii) dichloride dichloromethane complex (17 mg, 0.02 mmol), and potassium carbonate (70 mg, 0.5 mmol) were suspended in 5 ml (9:1 dioxane/water) in a sealed tube. Nitrogen gas was bubbled through the reaction solution for 2 minutes, then the vessel was sealed and heated at 110° C. for 3 hours. Reaction was cooled to rt and the mixture was diluted with 50 ml water and extracted with DCM (3×30 ml). The combined organics were concentrated under reduced pressure and the crude mixture was used for the next reaction without further purification.

Crude product from the prior step was dissolved in DCM (5 mL) and TFA (0.5 mL) was added. Reaction was stirred at RT for 4 hours, then solvent was concentrated under reduced pressure and crude product was purified by reverse phase HPLC to afford (S)-5-((((6-(2,2′-dichloro-3′-(5-methoxy-2-(((S)-5-oxopyrrolidin-2-yl)methyl)-1,2,3,4-tetrahydroisoquinolin-7-yl)-[1,1′-biphenyl]-3-yl)-2-methoxypyridin-3-yl)methyl)amino)methyl)pyrrolidin-2-one (29 mg, 19.7% yield) as a white powder (bis-formic acid salt). m/z: 715 [M+H]⁺ observed. ¹H NMR (400 MHz, CDCl₃) δ 7.76 (d, J=7.6 Hz, 1H), 7.62 (d, J=7.6 Hz, 1H), 7.48-7.25 (m, 6H), 6.81 (s, 1H), 6.74 (s, 1H), 4.02-3.61 (m, 12H), 2.89-2.76 (m, 6H), 2.68-2.53 (m, 2H), 2.40-2.22 (m, 6H), and 1.85-1.73 (m, 2H).

### Example 425: (S)-5-((6-(2,2′-Dichloro-3′-(6-methoxy-5-(((((S)-5-oxopyrrolidin-2-yl)methyl)-amino)-methyl)-pyridin-2-yl)-[1,1′-biphenyl]-3-yl)-8-fluoro-3,4-dihydroisoquinolin-2(1H)-yl)methyl)pyrrolidin-2-one

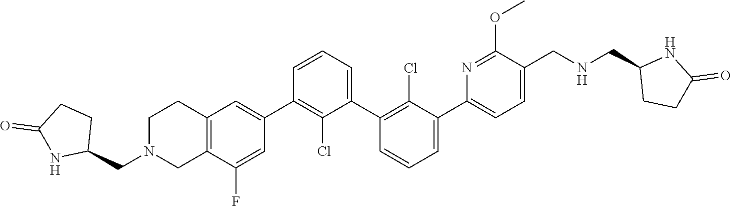 [SMILES: COc1nc(-c2cccc(-c3cccc(-c4cc(F)c5c(c4)CCN(C[C@@H]4CCC(=O)N4)C5)c3Cl)c2Cl)ccc1CNC[C@@H]1CCC(=O)N1]

### (S)-5-((6-Bromo-8-fluoro-3,4-dihydroisoquinolin-2(1H)-yl)methyl)pyrrolidin-2-one

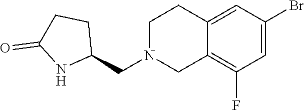 [SMILES: O=C1CC[C@@H](CN2CCc3cc(Br)cc(F)c3C2)N1]

A mixture of 6-bromo-8-fluoro-1,2,3,4-tetrahydroisoquinoline hydrochloride (75 mg, 0.33 mmol), (2S)-5-oxopyrrolidin-2-yl]methyl methanesulfonate (65 mg, 0.34 mmol), and potassium carbonate (70 mg, 0.5 mmol) in 3 mL acetonitrile were stirred at 80° C. for 12 hours. Reaction was filtered through CELITE®. The filtrate was concentrated under reduced pressure and the crude oil was purified by normal phase SiO₂ chromatography (0-100% EtOAc/hexane) to afford (S)-5-((6-bromo-8-fluoro-3,4-dihydroisoquinolin-2(1H)-yl)methyl)pyrrolidin-2-one (80 mg, 72.7% yield) as a white solid. m/z: 328 [M+H]⁺ observed.

### (S)-5-((6-(2,2′-Dichloro-3′-(6-methoxy-5-(((((S)-5-oxopyrrolidin-2-yl)methyl)-amino)-methyl)-pyridin-2-yl)-[1,1′-biphenyl]-3-yl)-8-fluoro-3,4-dihydroisoquinolin-2(1H)-yl)methyl)pyrrolidin-2-one

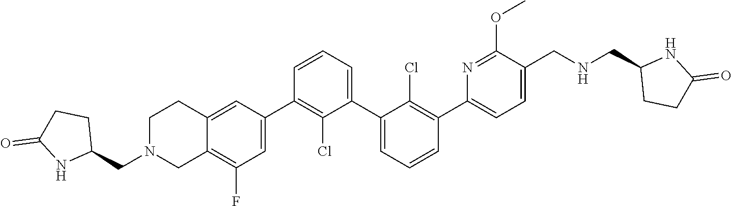 [SMILES: COc1nc(-c2cccc(-c3cccc(-c4cc(F)c5c(c4)CCN(C[C@@H]4CCC(=O)N4)C5)c3Cl)c2Cl)ccc1CNC[C@@H]1CCC(=O)N1]

A mixture of(5S)-5-[(2-chloro-7,8-dihydro-5H-1,6-naphthyridin-6-yl)methyl]pyrrolidin-2-one (65 mg, 0.20 mmol), tert-butyl N-[[6-[2-chloro-3-[2-chloro-3-(4,4,5,5-tetramethyl-1,3,2-dioxaborolan-2-yl)phenyl]phenyl]-2-methoxy-3-pyridyl]methyl]-N-[[(2S)-5-oxopyrrolidin-2-yl]methyl]carbamate (140 mg, 0.21 mmol), 1,1′-bis(diphenylphosphino)-ferrocene-palladium(ii) dichloride dichloromethane complex (17 mg, 0.02 mmol), and potassium carbonate (70 mg, 0.5 mmol) were suspended in 5 mL (9:1 dioxane/water) in a sealed tube. Nitrogen gas was bubbled through the reaction solution for 2 minutes, then the vessel was sealed and heated at 110° C. for 3 hours. Reaction was cooled to rt and the mixture was diluted with 50 ml water and extracted with DCM (3×30 mL). The combined organics were concentrated under reduced pressure and the crude mixture was used for the next reaction without further purification.

Crude product from the prior step was dissolved in DCM (5 mL) and TFA (0.5 mL) was added. Reaction was stirred at RT for 4 hours, then solvent was concentrated under reduced pressure and crude sample was purified by reverse phase HPLC to afford (S)-5-((6-(2,2′-dichloro-3′-(6-methoxy-5-(((((S)-5-oxopyrrolidin-2-yl)methyl)-amino)-methyl)-pyridin-2-yl)-[1,1′-biphenyl]-3-yl)-8-fluoro-3,4-dihydroisoquinolin-2(1H)-yl)methyl)pyrrolidin-2-one (28 mg, 20.1% yield) as a white powder (bis-formic acid salt). m/z: 703 [M+H]⁺ observed. ¹H NMR (400 MHz, CDCl₃) δ 7.87 (d, J=7.6 Hz 1H), 7.63 (dd, J=7.6 Hz and J=1.6 Hz, 1H), 7.51-7.31 (m, 6H), 7.04 (s, 1H), 6.98 (d, J=10 Hz, 1H), 4.28 (s, 2H), 4.08-4.02 (m, 5H), 3.87-3.71 (m, 2H), 3.22-3.01 (m, 2H), 2.99-2.64 (m, 6H), 2.44-2.26 (m, 6H) and 1.94-1.78 (m, 2H).

### Example 426: (S)-5-((((6-(2,2′-Dichloro-3′-(6-(((S)-5-oxopyrrolidin-2-yl)-methyl)-5,6,7,8-tetrahydro-1,6-naphthyridin-2-yl)-[1,1′-biphenyl]-3-yl)-2-methoxypyridin-3-yl)-methyl)-amino)-methyl)pyrrolidin-2-one

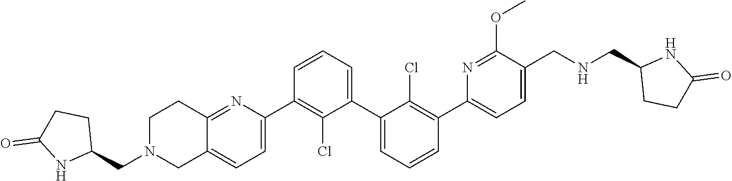 [SMILES: COc1nc(-c2cccc(-c3cccc(-c4ccc5c(n4)CCN(C[C@@H]4CCC(=O)N4)C5)c3Cl)c2Cl)ccc1CNC[C@@H]1CCC(=O)N1]

### (S)-5-((2-Chloro-7,8-dihydro-1,6-naphthyridin-6(5H)-yl)methyl)pyrrolidin-2-one

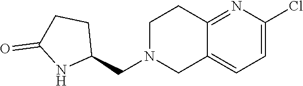 [SMILES: O=C1CC[C@@H](CN2CCc3nc(Cl)ccc3C2)N1]

A mixture of 2-chloro-5,6,7,8-tetrahydro-1,6-naphthyridine hydrochloride (70 mg, 0.42 mmol), (2S)-5-oxopyrrolidin-2-yl]-methyl methanesulfonate (80 mg, 0.41 mmol), and potassium carbonate (70 mg, 0.5 mmol) in 3 mL acetonitrile were stirred at 80° C. for 12 hours. Reaction was filtered through CELITE®. The filtrate was concentrated under reduced pressure and the crude oil was purified by normal phase SiO₂ chromatography (0-100% EtOAc/hexane) to afford (S)-5-((2-chloro-7,8-dihydro-1,6-naphthyridin-6(5H)-yl)methyl)pyrrolidin-2-one (85 mg, 77.3% yield as a white solid. m/z: 266 [M+H]⁺ observed.

### (S)-5-((((6-(2,2′-Dichloro-3′-(6-(((S)-5-oxopyrrolidin-2-yl)-methyl)-5,6,7,8-tetrahydro-1,6-naphthyridin-2-yl)-[1,1′-biphenyl]-3-yl)-2-methoxypyridin-3-yl)-methyl)-amino)-methyl)pyrrolidin-2-one

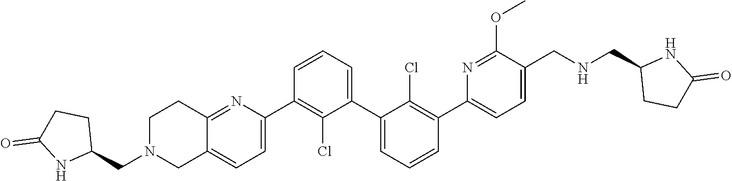 [SMILES: COc1nc(-c2cccc(-c3cccc(-c4ccc5c(n4)CCN(C[C@@H]4CCC(=O)N4)C5)c3Cl)c2Cl)ccc1CNC[C@@H]1CCC(=O)N1]

A mixture of(5S)-5-[(2-chloro-7,8-dihydro-5H-1,6-naphthyridin-6-yl)methyl]pyrrolidin-2-one (55 mg, 0.21 mmol), tert-butyl N-[[6-[2-chloro-3-[2-chloro-3-(4,4,5,5-tetramethyl-1,3,2-dioxaborolan-2-yl)phenyl]phenyl]-2-methoxy-3-pyridyl]methyl]-N-[[(2S)-5-oxopyrrolidin-2-yl]methyl]carbamate (140 mg, 0.21 mmol), 1,1′-bis(diphenylphosphino)-ferrocene-palladium(ii) dichloride dichloromethane complex (17 mg, 0.02 mmol), and potassium carbonate (70 mg, 0.5 mmol) were suspended in 5 mL (9:1 dioxane/water) in a sealed tube. Nitrogen gas was bubbled through the reaction solution for 2 minutes, then the vessel was sealed and heated at 110° C. for 3 hours. Reaction was cooled to rt and the mixture was diluted with 50 mL water and extracted with DCM (3×30 ml). The combined organics were concentrated under reduced pressure and the crude mixture was used for the next reaction without further purification.

Crude product from the prior step was dissolved in DCM (5 mL) and TFA (0.5 mL) was added. Reaction was stirred at rt for 4 hours, then solvent was concentrated under reduced pressure and crude sample was purified by reverse phase HPLC to afford (S)-5-((((6-(2,2′-dichloro-3′-(6-(((S)-5-oxopyrrolidin-2-yl)-methyl)-5,6,7,8-tetrahydro-1,6-naphthyridin-2-yl)-[1,1′-biphenyl]-3-yl)-2-methoxypyridin-3-yl)-methyl)-amino)-methyl)pyrrolidin-2-one (28 mg, 19.7% yield) as a white powder (bis-formic acid salt). m/z: 686 [M+H]⁺ observed. ¹H NMR (400 MHz, CDCl₃) δ 7.81 (d, J=8.0 Hz 1H), 7.64-7.60 (m, 2H), 7.51-7.29 (m, 7H), 4.10-3.72 (m, 9H), 3.08-2.89 (m, 6H), 2.71-2.59 (m, 2H), 2.42-2.27 (m, 6H) and 1.90-1.78 (m, 2H).

### Example 427: (S)-5-((8-Chloro-7-(2,2′-dichloro-3′-(6-methoxy-5-(((((S)-5-oxopyrrolidin-2-yl)methyl)-amino)-methyl)-pyridin-2-yl)-[1,1′-biphenyl]-3-yl)-3,4-dihydroisoquinolin-2(1H)-yl)methyl)pyrrolidin-2-one

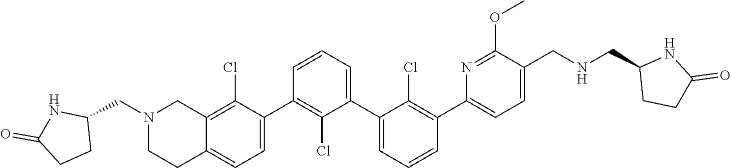 [SMILES: COc1nc(-c2cccc(-c3cccc(-c4ccc5c(c4Cl)CN(C[C@@H]4CCC(=O)N4)CC5)c3Cl)c2Cl)ccc1CNC[C@@H]1CCC(=O)N1]

### (S)-5-((7-Bromo-8-chloro-3,4-dihydroisoquinolin-2(1H)-yl)methyl)pyrrolidin-2-one

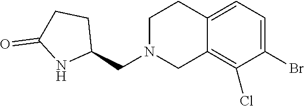 [SMILES: O=C1CC[C@@H](CN2CCc3ccc(Br)c(Cl)c3C2)N1]

A mixture of 7-bromo-8-chloro-1,2,3,4-tetrahydroisoquinoline hydrochloride (75 mg, 0.3 mmol), (2S)-5-oxopyrrolidin-2-yl]methyl methanesulfonate (80 mg, 0.4 mmol), and potassium carbonate (70 mg, 0.5 mmol) in 3 mL acetonitrile were stirred at 80° C. for 12 hours. Reaction was filtered through CELITE®. The filtrate was concentrated under reduced pressure and the crude oil was purified by normal phase SiO₂ chromatography (0-100% EtOAc/hexane) to afford (S)-5-((7-bromo-8-chloro-3,4-dihydroisoquinolin-2(1H)-yl)methyl)pyrrolidin-2-one (80 mg, 75% yield as a white solid. m/z: 344 [M+H]⁺ observed.

### (S)-5-((8-Chloro-7-(2,2′-dichloro-3′-(6-methoxy-5-(((((S)-5-oxopyrrolidin-2-yl)methyl)-amino)-methyl)-pyridin-2-yl)-[1,1′-biphenyl]-3-yl)-3,4-dihydroisoquinolin-2(1H)-yl)methyl)pyrrolidin-2-one

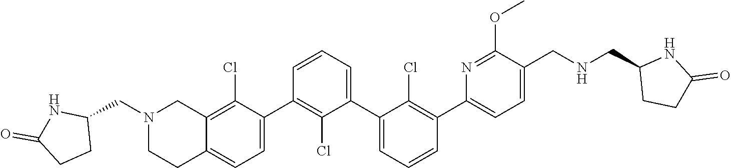 [SMILES: COc1nc(-c2cccc(-c3cccc(-c4ccc5c(c4Cl)CN(C[C@@H]4CCC(=O)N4)CC5)c3Cl)c2Cl)ccc1CNC[C@@H]1CCC(=O)N1]

A mixture of(5S)-5-[(2-chloro-7,8-dihydro-5H-1,6-naphthyridin-6-yl)methyl]pyrrolidin-2-one (70 mg, 0.2 mmol), tert-butyl N-[[6-[2-chloro-3-[2-chloro-3-(4,4,5,5-tetramethyl-1,3,2-dioxaborolan-2-yl)phenyl]phenyl]-2-methoxy-3-pyridyl]methyl]-N-[[(2S)-5-oxopyrrolidin-2-yl]methyl]carbamate (140 mg, 0.21 mmol), 1,1′-bis(diphenylphosphino)-ferrocene-palladium(ii) dichloride dichloromethane complex (17 mg, 0.02 mmol), and potassium carbonate (70 mg, 0.5 mmol) were suspended in 5 mL (9:1 dioxane/water) in a sealed tube. Nitrogen gas was bubbled through the reaction solution for 2 minutes, then the vessel was sealed and heated at 110° C. for 3 hours. Reaction was cooled to rt and the mixture was diluted with 50 ml water and extracted with DCM (3×30 mL). The combined organics were concentrated under reduced pressure and the crude mixture was used for the next reaction without further purification.

Crude product from the prior step was dissolved in DCM (5 mL) and TFA (0.5 mL) was added. Reaction was stirred at RT for 4 hours, then solvent was concentrated under reduced pressure and crude sample was purified by reverse phase HPLC to afford (S)-5-((8-chloro-7-(2,2′-dichloro-3′-(6-methoxy-5-(((((S)-5-oxopyrrolidin-2-yl)methyl)-amino)-methyl)-pyridin-2-yl)-[1,1′-biphenyl]-3-yl)-3,4-dihydroisoquinolin-2(1H)-yl)methyl)pyrrolidin-2-one (26 mg, 17.8% yield) as a white powder (bis-formic acid salt). m/z: 720 [M+H]⁺ observed. ¹H NMR (400 MHz, CDCl₃) δ 7.84 (d, J=7.6 Hz 1H), 7.62 (d, J=7.6 Hz, 1H), 7.50-7.26 (m, 6H), 7.18-7.11 (m, 2H), 4.22 (s, 2H), 4.06-3.95 (m, 5H), 3.88-3.65 (m, 2H), 3.13 (s, 2H), 2.99-2.62 (m, 6H), 2.40-2.24 (m, 6H) and 1.92-1.78 (m, 2H).

### Example 428: (S)-5-((7-(2,2′-Dichloro-3′-(6-methoxy-5-(((((S)-5-oxopyrrolidin-2-yl)methyl)-amino)methyl)-pyridin-2-yl)-[1,1′-biphenyl]-3-yl)-8-fluoro-3,4-dihydroisoquinolin-2(1H)-yl)methyl)-pyrrolidin-2-one

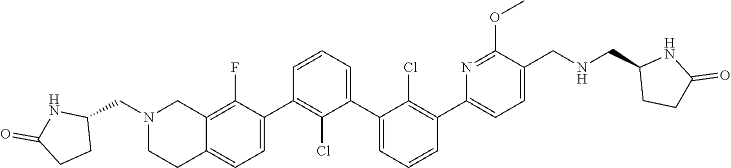 [SMILES: COc1nc(-c2cccc(-c3cccc(-c4ccc5c(c4F)CN(C[C@@H]4CCC(=O)N4)CC5)c3Cl)c2Cl)ccc1CNC[C@@H]1CCC(=O)N1]

### (S)-5-((7-Bromo-8-fluoro-3,4-dihydroisoquinolin-2(1H)-yl)methyl)pyrrolidin-2-one

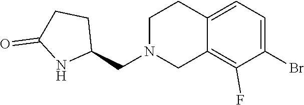 [SMILES: O=C1CC[C@@H](CN2CCc3ccc(Br)c(F)c3C2)N1]

A mixture of 7-bromo-8-fluoro-1,2,3,4-tetrahydroisoquinoline hydrochloride (75 mg, 0.3 mmol), (2S)-5-oxopyrrolidin-2-yl]methyl methanesulfonate (80 mg, 0.4 mmol), and potassium carbonate (70 mg, 0.5 mmol) in 3 mL acetonitrile were stirred at 80° C. for 12 hours. Reaction was filtered through CELITE®. The filtrate was concentrated under reduced pressure and the crude oil was purified by normal phase SiO₂ chromatography (0-100% EtOAc/hexane) to afford (80 mg, 78.7% yield as a white solid. m/z: 328 [M+H]⁺ observed.

### (S)-5-((7-(2,2′-Dichloro-3′-(6-methoxy-5-(((((S)-5-oxopyrrolidin-2-yl)methyl)-amino)methyl)-pyridin-2-yl)-[1,1′-biphenyl]-3-yl)-8-fluoro-3,4-dihydroisoquinolin-2(1H)-yl)methyl)-pyrrolidin-2-one

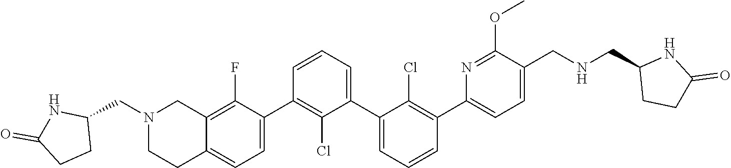 [SMILES: COc1nc(-c2cccc(-c3cccc(-c4ccc5c(c4F)CN(C[C@@H]4CCC(=O)N4)CC5)c3Cl)c2Cl)ccc1CNC[C@@H]1CCC(=O)N1]

A mixture of (5S)-5-[(2-chloro-7,8-dihydro-5H-1,6-naphthyridin-6-yl)methyl]pyrrolidin-2-one (65 mg, 0.2 mmol), tert-butyl N-[[6-[2-chloro-3-[2-chloro-3-(4,4,5,5-tetramethyl-1,3,2-dioxaborolan-2-yl)phenyl]phenyl]-2-methoxy-3-pyridyl]methyl]-N-[[(2S)-5-oxopyrrolidin-2-yl]methyl]carbamate (140 mg, 0.21 mmol), 1,1′-bis(diphenylphosphino)-ferrocene-palladium(ii) dichloride dichloromethane complex (17 mg, 0.02 mmol), and potassium carbonate (70 mg, 0.5 mmol) were suspended in 5 mL (9:1 dioxane/water) in a sealed tube. Nitrogen gas was bubbled through the reaction solution for 2 minutes, then the vessel was sealed and heated at 110° C. for 3 hours. Reaction was cooled to rt and the mixture was diluted with 50 ml water and extracted with DCM (3×3 mL). The combined organics were concentrated under reduced pressure and the crude mixture was used for the next reaction without further purification.

Crude product from the prior step was dissolved in DCM (5 mL) and TFA (0.5 mL) was added. Reaction was stirred at RT for 4 hours, then solvent was concentrated under reduced pressure and crude sample was purified by reverse phase HPLC to afford (S)-5-((7-(2,2′-dichloro-3′-(6-methoxy-5-(((((S)-5-oxopyrrolidin-2-yl)methyl)-amino)methyl)-pyridin-2-yl)-[1,1′-biphenyl]-3-yl)-8-fluoro-3,4-dihydroisoquinolin-2(1H)-yl)methyl)-pyrrolidin-2-one (25 mg, 17.9% yield) as a white powder (bis-formic acid salt). m/z: 703 [M+H]⁺ observed. ¹H NMR (400 MHz, CDCl₃) δ 7.82 (d, J=7.6 Hz 1H), 7.62 (dd, J=7.6 Hz and J=1.6 Hz, 1H), 7.50-7.30 (m, 6H), 7.13 (t, J=7.6 Hz, 1H), 7.04 (d, J=7.6 Hz 1H), 4.15-3.97 (m, 7H), 3.83-3.67 (m, 2H), 3.07-2.60 (m, 8H), 2.40-2.26 (m, 6H) and 1.85-1.45 (m, 2H).

### Example 443: (S)-5-((((6-(2,2′-Dichloro-3′-(6-(((S)-5-oxopyrrolidin-2-yl)methyl)-5,6,7,8-tetrahydropyrido[4,3-d]pyrimidin-2-yl)-[1,1′-biphenyl]-3-yl)-2-methoxypyridin-3-yl)methyl)amino)methyl)pyrrolidin-2-one

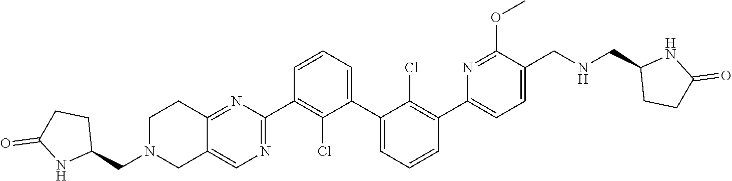 [SMILES: COc1nc(-c2cccc(-c3cccc(-c4ncc5c(n4)CCN(C[C@@H]4CCC(=O)N4)C5)c3Cl)c2Cl)ccc1CNC[C@@H]1CCC(=O)N1]

### (S)-5-((2-Chloro-7,8-dihydropyrido[4,3-d]pyrimidin-6(5H)-yl)methyl)pyrrolidin-2-one

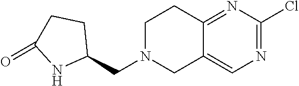 [SMILES: O=C1CC[C@@H](CN2CCc3nc(Cl)ncc3C2)N1]

A mixture of 2-chloro-5,6,7,8-tetrahydropyrido[4,3-d]pyrimidine hydrochloride (70 mg, 0.41 mmol), (2S)-5-oxopyrrolidin-2-yl]methyl methanesulfonate (80 mg, 0.41 mmol), and potassium carbonate (70 mg, 0.5 mmol) in 3 mL acetonitrile were stirred at 80° C. for 12 hours. Reaction was filtered through CELITE®. The filtrate was concentrated under reduced pressure and the crude oil was purified by normal phase SiO₂ chromatography (0-100% EtOAc/hexane) to afford (S)-5-((2-chloro-7,8-dihydropyrido[4,3-d]pyrimidin-6(5H)-yl)methyl)pyrrolidin-2-one (85 mg, 77% yield as a white solid. m/z: 267 [M+H]⁺ observed.

### (S)-5-((((6-(2,2′-Dichloro-3′-(6-(((S)-5-oxopyrrolidin-2-yl)methyl)-5,6,7,8-tetrahydropyrido[4,3-d]pyrimidin-2-yl)-[1,1′-biphenyl]-3-yl)-2-methoxypyridin-3-yl)methyl)amino)methyl)pyrrolidin-2-one

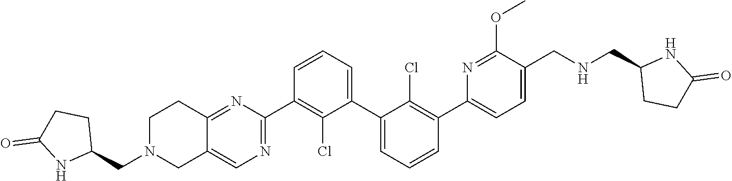 [SMILES: COc1nc(-c2cccc(-c3cccc(-c4ncc5c(n4)CCN(C[C@@H]4CCC(=O)N4)C5)c3Cl)c2Cl)ccc1CNC[C@@H]1CCC(=O)N1]

A mixture of (5S)-5-[(2-chloro-7,8-dihydro-5H-pyrido[4,3-d]pyrimidin-6-yl)methyl]pyrrolidin-2-one (55 mg, 0.21 mmol), tert-butyl N-[[6-[2-chloro-3-[2-chloro-3-(4,4,5,5-tetramethyl-1,3,2-dioxaborolan-2-yl)phenyl]phenyl]-2-methoxy-3-pyridyl]methyl]-N-[[(2S)-5-oxopyrrolidin-2-yl]methyl]carbamate (140 mg, 0.21 mmol), 1,1′-bis(diphenylphosphino)-ferrocene-palladium(ii) dichloride dichloromethane complex (17 mg, 0.02 mmol), and potassium carbonate (70 mg, 0.5 mmol) were suspended in 5 mL (9:1 dioxane/water) in a sealed tube. Nitrogen gas was bubbled through the reaction solution for 2 minutes, then the vessel was sealed and heated at 110° C. for 3 hours. Reaction was cooled to rt and the mixture was diluted with 50 mL water and extracted with DCM (3×30 mL). The combined organics were concentrated under reduced pressure and the crude mixture was used for the next reaction without further purification.

Crude product from the prior step was dissolved in DCM (5 mL) and TFA (0.5 mL) was added. Reaction was stirred at RT for 4 hours, then solvent was concentrated under reduced pressure and crude sample was purified by reverse phase HPLC to afford (S)-5-((((6-(2,2′-dichloro-3′-(6-(((S)-5-oxopyrrolidin-2-yl)methyl)-5,6,7,8-tetrahydropyrido[4,3-d]pyrimidin-2-yl)-[1,1′-biphenyl]-3-yl)-2-methoxypyridin-3-yl)methyl)amino)methyl)pyrrolidin-2-one (14 mg, 9.9% yield) as a white powder (bis-formic acid salt). m/z: 687 [M+H]⁺ observed. ¹H NMR (400 MHz, CDCl₃) δ 8.61 (s, 1H), 8.45 (s, 1H), 7.84 (d, J=7.6 Hz 1H), 7.65-7.60 (m, 2H), 7.55-7.45 (m, 3H), 7.37 (dd, J=7.6 Hz and J=1.6 Hz, 1H), 7.28 (d, J=7.6 Hz 1H), 4.03-3.73 (m, 9H), 3.09-3.01 (m, 3H), 2.93-2.85 (m, 3H), 2.72-2.61 (m, 2H), 2.39-2.26 (m, 6H) and 1.90-1.78 (m, 2H).

### Example 471: (S)-5-((((6-(2,2′-Dichloro-3′-(4-methoxy-6-(((S)-5-oxopyrrolidin-2-yl)methyl)-5,6,7,8-tetrahydropyrido[4,3-d]pyrimidin-2-yl)-[1,1′-biphenyl]-3-yl)-2-methoxypyridin-3-yl)methyl)amino)methyl)pyrrolidin-2-one

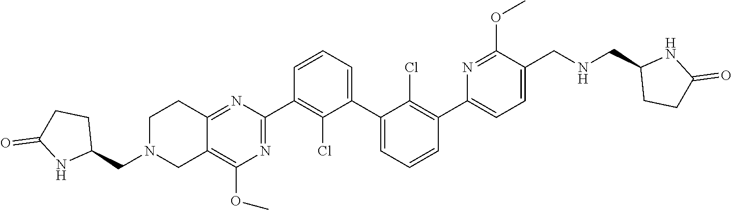 [SMILES: COc1nc(-c2cccc(-c3cccc(-c4nc5c(c(OC)n4)CN(C[C@@H]4CCC(=O)N4)CC5)c3Cl)c2Cl)ccc1CNC[C@@H]1CCC(=O)N1]

### (S)-5-((2-Chloro-4-methoxy-7,8-dihydropyrido[4,3-d]pyrimidin-6(5H)-yl)methyl)pyrrolidin-2-one

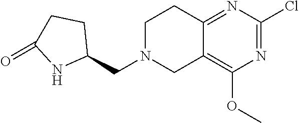 [SMILES: COc1nc(Cl)nc2c1CN(C[C@@H]1CCC(=O)N1)CC2]

A mixture of 2,4-dichloro-5,6,7,8-tetrahydropyrido[4,3-d]pyrimidine hydrochloride (60 mg, 0.3 mmol), (2S)-5-oxopyrrolidin-2-yl]methyl methanesulfonate (60 mg, 0.31 mmol), and potassium carbonate (70 mg, 0.5 mmol) in 3 mL acetonitrile were stirred at 80° C. for 12 hours. Reaction was filtered through CELITE®. The filtrate was concentrated under reduced pressure and the crude oil was purified by normal phase SiO₂ chromatography (0-100% EtOAc/hexane) to afford (S)-5-((2-chloro-4-methoxy-7,8-dihydropyrido[4,3-d]pyrimidin-6(5H)-yl)methyl)pyrrolidin-2-one (55 mg, 59.7% yield) as a white solid. m/z: 297 [M+H]⁺ observed.

### (S)-5-((((6-(2,2′-Dichloro-3′-(4-methoxy-6-(((S)-5-oxopyrrolidin-2-yl)methyl)-5,6,7,8-tetrahydropyrido[4,3-d]pyrimidin-2-yl)-[1,1′-biphenyl]-3-yl)-2-methoxypyridin-3-yl)methyl)amino)methyl)pyrrolidin-2-one

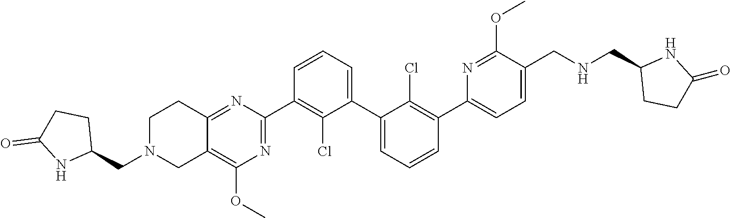 [SMILES: COc1nc(-c2cccc(-c3cccc(-c4nc5c(c(OC)n4)CN(C[C@@H]4CCC(=O)N4)CC5)c3Cl)c2Cl)ccc1CNC[C@@H]1CCC(=O)N1]

A mixture of(5S)-5-[(2-chloro-4-methoxy-7,8-dihydro-5H-pyrido[4,3-d]pyrimidin-6-yl)methyl]pyrrolidin-2-one (45 mg, 0.15 mmol), tert-butyl N-[[6-[2-chloro-3-[2-chloro-3-(4,4,5,5-tetramethyl-1,3,2-dioxaborolan-2-yl)phenyl]phenyl]-2-methoxy-3-pyridyl]methyl]-N-[[(2S)-5-oxopyrrolidin-2-yl]methyl]carbamate (70 mg, 1.1 mmol), 1,1′-bis(diphenylphosphino)-ferrocene-palladium(II)dichloride dichloromethane complex (17 mg, 0.02 mmol), and potassium carbonate (70 mg, 0.5 mmol) were suspended in 5 mL (9:1 dioxane/water) in a sealed tube. Nitrogen gas was bubbled through the reaction solution for 2 minutes, then the vessel was sealed and heated at 110° C. for 3 hours. Reaction was cooled to rt and the mixture was diluted with 50 mL water and extracted with DCM (3×30 mL). The combined organics were concentrated under reduced pressure and the crude mixture was used for the next reaction without further purification.

Crude product from the prior step was dissolved in DCM (5 mL) and TFA (0.5 mL) was added. Reaction was stirred at RT for 4 hours, then solvent was concentrated under reduced pressure and crude sample was purified by reverse phase HPLC to afford (S)-5-((((6-(2,2′-dichloro-3′-(4-methoxy-6-(((S)-5-oxopyrrolidin-2-yl)methyl)-5,6,7,8-tetrahydropyrido[4,3-d]pyrimidin-2-yl)-[1,1′-biphenyl]-3-yl)-2-methoxypyridin-3-yl)methyl)amino)methyl)pyrrolidin-2-one (15 mg, 20.7% yield) as a white powder (bis-formic acid salt). m/z: 717 [M+H]⁺ observed. ¹H NMR (400 MHz, CDCl₃) δ 8.15 (s, 1H), 7.87 (dd, J=8.0 Hz and J=3.2 Hz, 1H), 7.66-7.63 (m, 2H), 7.53-7.33 (m, 5H), 4.28 (s, 2H), 4.09-3.98 (m, 8H), 3.73-3.31 (m, 2H), 3.20-3.17 (m, 2H), 2.99-2.89 (m, 3H), 2.87-2.83 (m, 1H), 2.71-2.63 (m, 2H), 2.45-2.28 (m, 6H) and 1.95-1.78 (m, 2H).

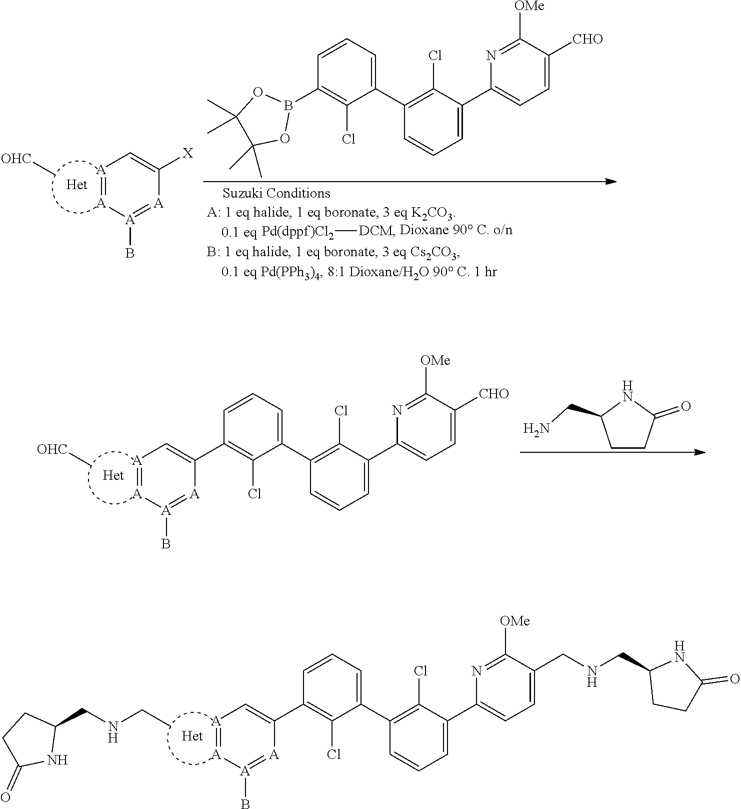

### Example 459: (S)-5-((((6-(2,2′-Dichloro-3′-(2-(((((S)-5-oxopyrrolidin-2-yl)methyl)amino)methyl)furo[2,3-b]pyridin-5-yl)-[1,1′-biphenyl]-3-yl)-2-methoxypyridin-3-yl)methyl)amino)methyl)pyrrolidin-2-one

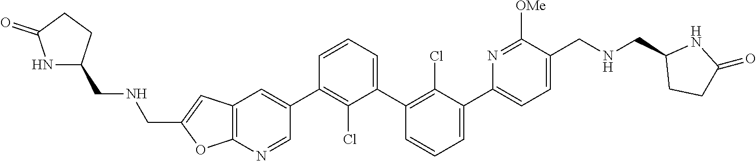 [SMILES: COc1nc(-c2cccc(-c3cccc(-c4cnc5oc(CNC[C@@H]6CCC(=O)N6)cc5c4)c3Cl)c2Cl)ccc1CNC[C@@H]1CCC(=O)N1]

### 5-(2,2′-Dichloro-3′-(5-formyl-6-methoxypyridin-2-yl)-[1,1′-biphenyl]-3-yl)furo[2,3-b]pyridine-2-carbaldehyde

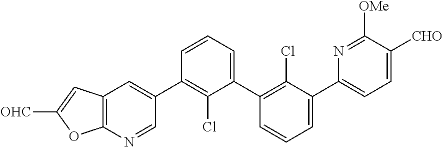 [SMILES: COc1nc(-c2cccc(-c3cccc(-c4cnc5oc(C=O)cc5c4)c3Cl)c2Cl)ccc1C=O]

Suzuki Condition B: A microwave vial was charged with 5-chlorofuro[2,3-b]pyridine-2-carbaldehyde (0.03 g, 0.019 mmol), 6-(2,2′-dichloro-3′-(4,4,5,5-tetramethyl-1,3,2-dioxaborolan-2-yl)-[1,1′-biphenyl]-3-yl)-2-methoxynicotinaldehyde (0.06 g, 0.12 mmol), Cs₂CO₃ (0.12 g 0.37 mmol), and Pd(PPh₃)₄ (0.03 g, 0.02 mmol). Dioxane was added (2 ml), and the reaction was sparged with nitrogen, then 2 mL of water added. The mixture was stirred at 90° C. for 1 hr. The mixture was filtered through CELITE® and the filter cake was washed with EtOAc (3×15 mL). The filtrate was concentrated under reduced pressure. The residue was purified by normal phase flash SiO₂ chromatography using a gradient (0-5% methanol DCM) to afford 5-(2,2′-dichloro-3′-(5-formyl-6-methoxypyridin-2-yl)-[1,1′-biphenyl]-3-yl)furo[2,3-b]pyridine-2-carbaldehyde (0.062 g,100% yield). MS: m/z found 503 [M+H]⁺.

### (S)-5-((((6-(2,2′-Dichloro-3′-(2-(((((S)-5-oxopyrrolidin-2-yl)methyl)amino)methyl)furo[2,3-b]pyridin-5-yl)-[1,1′-biphenyl]-3-yl)-2-methoxypyridin-3-yl)methyl)amino)methyl)pyrrolidin-2-one

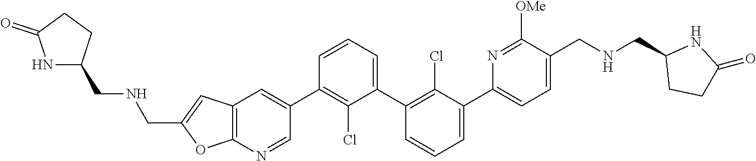 [SMILES: COc1nc(-c2cccc(-c3cccc(-c4cnc5oc(CNC[C@@H]6CCC(=O)N6)cc5c4)c3Cl)c2Cl)ccc1CNC[C@@H]1CCC(=O)N1]

To a mixture of 5-(2,2′-dichloro-3′-(5-formyl-6-methoxypyridin-2-yl)-[1,1′-biphenyl]-3-yl)furo[2,3-b]pyridine-2-carbaldehyde (0.09 g, 0.18 mmol, 1 eq) and (S)-5-(aminomethyl)pyrrolidin-2-one (0.06 g, 0.54 mmol) in 1:1 THF/MeOH (6 mL) was added acetic acid (0.03 g, 0.54 mmol) and 4 A molecular sieve (1 g). The mixture was stirred at r.t. for 3 hrs. Sodium cyanoborohydride (0.04 g, 0.63 mmol) was added and the mixture was stirred at r.t. for 1 hr. The reaction was concentrated, and residue purified by semi-prep HPLC (with 0.05% Formic acid modifier). ACN was evaporated off, and aqueous portion was lyophilized to give 8.0 mg (7%) of the product as bis formic acid salt affording (S)-5-((((6-(2,2′-dichloro-3′-(2-(((((S)-5-oxopyrrolidin-2-yl)methyl)amino)methyl)furo[2,3-b]pyridin-5-yl)-[1,1′-biphenyl]-3-yl)-2-methoxypyridin-3-yl)methyl)amino)methyl)pyrrolidin-2-one as a white solid. MS: m/z found 699 [M+H]⁺; ¹H NMR (400 MHz, Methanol-d₄) δ 8.38 (s, 2H), 8.26 (d, J=2.1, 0.7 Hz, 1H), 8.10 (d, J=2.1, 0.7 Hz, 1H), 7.77 (d, J=7.5 Hz, 1H), 7.62 (d, 1H), 7.56-7.43 (m, 3H), 7.43-7.33 (m, 2H), 7.27 (d, J=7.5 Hz, 1H), 6.84 (s, 1H), 4.02 (s, 7H), 3.91 (t, 1H), 3.81 (q, J=6.6 Hz, 1H), 2.98-2.84 (m, 2H), 2.82-2.66 (m, 2H), 2.41-2.17 (m, 6H), 1.90-1.74 (m, 2H).

### Example 434: (S)-5-((((6-(2,2′-Dichloro-3′-(3-(((((S)-5-oxopyrrolidin-2-yl)methyl)amino)methyl)-1H-indazol-5-yl)-[1,1′-biphenyl]-3-yl)-2-methoxypyridin-3-yl)methyl)amino)methyl)pyrrolidin-2-one

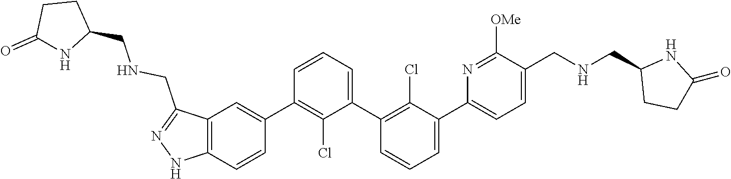 [SMILES: COc1nc(-c2cccc(-c3cccc(-c4ccc5[nH]nc(CNC[C@@H]6CCC(=O)N6)c5c4)c3Cl)c2Cl)ccc1CNC[C@@H]1CCC(=O)N1]

This compound was prepared in a similar fashion to Example 459, using Suzuki condition B, with 5-bromo-1H-indazole-3-carbaldehyde and 6-(2,2′-dichloro-3′-(4,4,5,5-tetramethyl-1,3,2-dioxaborolan-2-yl)-[1,1′-biphenyl]-3-yl)-2-methoxynicotinaldehyde. MS: m/z found 698 [M+H]⁺; ¹H NMR (400 MHz, Methanol-d₄) δ 8.59 (d, J=2.0 Hz, 1H), 8.44 (d, J=2.1 Hz, 1H), 8.41 (s, 3H), 7.76 (d, J=7.5 Hz, 1H), 7.62 (dd, J=7.7, 1.7 Hz, 1H), 7.53-7.47 (m, 3H), 7.43-7.34 (m, 2H), 7.26 (d, J=7.4 Hz, 1H), 4.32-4.17 (m, 2H), 4.00 (d, J=16.6 Hz, 6H), 3.89 (s, 1H), 3.86-3.78 (m, 1H), 2.93-2.73 (m, 4H), 2.41-2.17 (m, 6H), 1.87-1.73 (m, 1H).

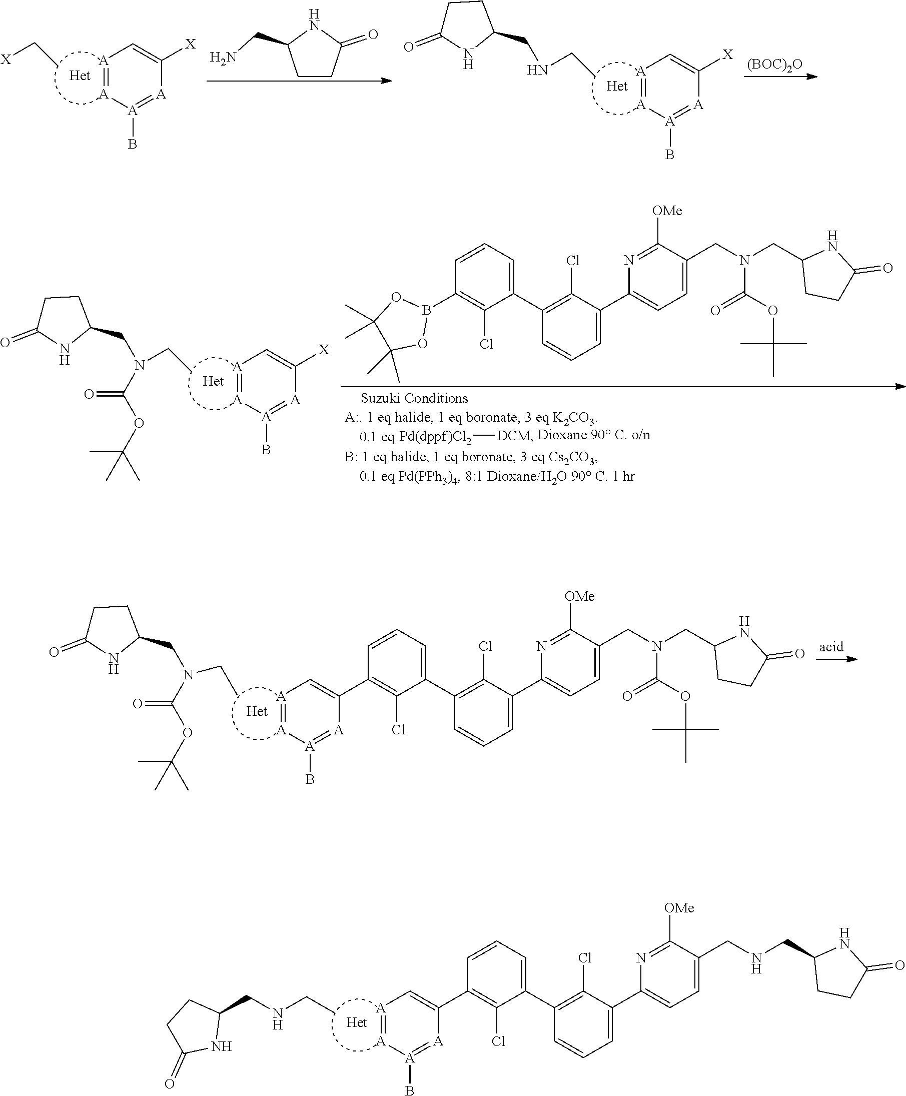

### Example 433: 6-(2,2′-Dichloro-3′-(6-methoxy-5-(((((S)-5-oxopyrrolidin-2-yl)methyl)amino)methyl)pyridin-2-yl)-[1,1′-biphenyl]-3-yl)-2-(((((S)-5-oxopyrrolidin-2-yl)methyl)amino)methyl)pyrido[2,3-d]pyrimidin-4(1H)-one

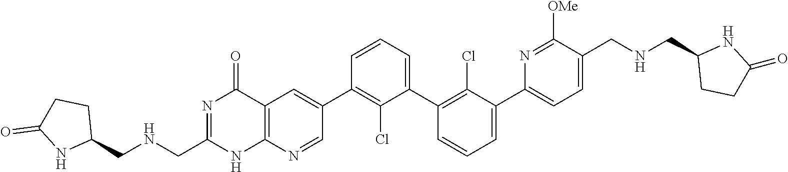 [SMILES: COc1nc(-c2cccc(-c3cccc(-c4cnc5[nH]c(CNC[C@@H]6CCC(=O)N6)nc(=O)c5c4)c3Cl)c2Cl)ccc1CNC[C@@H]1CCC(=O)N1]

### (S)-6-Bromo-2-((((5-oxopyrrolidin-2-yl)methyl)amino)methyl)pyrido[2,3-d]pyrimidin-4(1H)-one

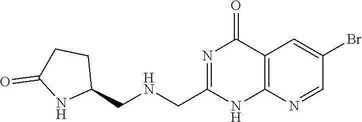 [SMILES: O=C1CC[C@@H](CNCc2nc(=O)c3cc(Br)cnc3[nH]2)N1]

A microwave vial was charged with 6-bromo-2-(chloromethyl)pyrido[2,3-d]pyrimidin-4(1H)-one (0.20 g, 0.73 mmol) and (S)-5-(aminomethyl)pyrrolidin-2-one (0.08 g, 0.73 mmol). Dioxane was added (0.5 ml), and the reaction was stirred at 125° C. for 1 hr in a microwave. The mixture was diluted with 50 ml EtOAc, then washed with saturated aqueous brine solution (2×30 mL) then dried over Na₂SO₄ and concentrated under reduced pressure. The residue was purified by normal phase flash SiO₂ chromatography using a linear gradient (0-10% MeOH/DCM) to afford (S)-6-bromo-2-((((5-oxopyrrolidin-2-yl)methyl)amino)methyl) pyrido[2,3-d]pyrimidin-4(1H)-one (0.13 g, 51% yield). MS: m/z found 352 [M+H]⁺.

### tert-Butyl (S)-((6-bromo-4-oxo-1,4-dihydropyrido[2,3-d]pyrimidin-2-yl)methyl)((5-oxopyrrolidin-2-yl)methyl)carbamate

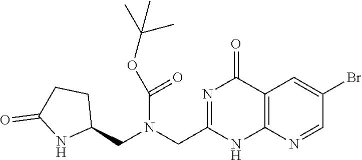 [SMILES: CC(C)(C)OC(=O)N(Cc1nc(=O)c2cc(Br)cnc2[nH]1)C[C@@H]1CCC(=O)N1]

A scintillation vial was charged with (S)-6-bromo-2-((((5-oxopyrrolidin-2-yl)methyl) amino)methyl) pyrido[2,3-d]pyrimidin-4(1H)-one (0.20 g, 0.73 mmol), Di-tert-butyl dicarbonate (0.20 g, 0.91 mmol), and triethylamine (0.73 ml, 0.91 mmol). THE was added (5 ml), and the reaction was stirred at room temp overnight. The mixture was diluted with 50 ml EtOAc, then washed with saturated aqueous brine solution (2×30 mL) then dried over Na₂SO₄ and concentrated under reduced pressure. The residue was purified by normal phase flash SiO₂ chromatography using a gradient (0-5% MeOH/DCM) to afford tert-butyl (S)-((6-bromo-4-oxo-1,4-dihydropyrido[2,3-d]pyrimidin-2-yl)methyl)((5-oxopyrrolidin-2-yl)methyl)carbamate (0.162 g, 79% yield). MS: m/z found 452 [M+H]⁺. ¹H NMR (400 MHz, Chloroform-d) δ 8.53 (s, 0.5 H), 8.32 (s, 1H), 8.03 (s, 0.5 H), 7.80 (d, 1H), 7.57 (d, J=8.6 Hz, 1H), 4.39-4.33 (m, 1H), 3.94 (s, 0.5H), 3.69 (d, J=13.8 Hz, 0.5H), 3.55-3.44 (m, 3H), 3.29 (s, 1H), 2.40 (s, 2H), 2.23 (s, 1H), 1.78 (s, 1H), 1.26 (s, 9H).

### tert-Butyl ((6-(3′-(2-(((tert-butoxycarbonyl)(((S)-5-oxopyrrolidin-2-yl)methyl)amino) methyl)-4-oxo-1,4-dihydropyrido[2,3-d]pyrimidin-6-yl)-2,2′-dichloro-[1,1′-biphenyl]-3-yl)-2-methoxypyridin-3-yl)methyl)(((S)-5-oxopyrrolidin-2-yl)methyl)carbamate

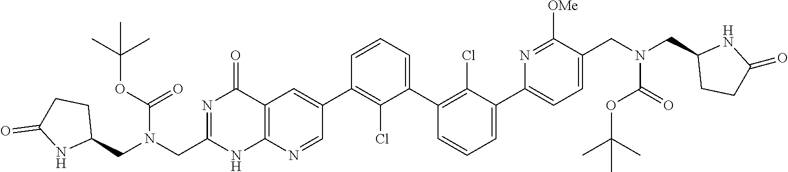 [SMILES: COc1nc(-c2cccc(-c3cccc(-c4cnc5[nH]c(CN(C[C@@H]6CCC(=O)N6)C(=O)OC(C)(C)C)nc(=O)c5c4)c3Cl)c2Cl)ccc1CN(C[C@@H]1CCC(=O)N1)C(=O)OC(C)(C)C]

A microwave vial was charged with tert-butyl (S)-((6-bromo-4-oxo-1,4-dihydropyrido[2,3-d]pyrimidin-2-yl)methyl)((5-oxopyrrolidin-2-yl)methyl)carbamate (0.16 g, 0.35 mmol), tert-butyl (S)-((6-(2,2′-dichloro-3′-(4,4,5,5-tetramethyl-1,3,2-dioxaborolan-2-yl)-[1,1′-biphenyl]-3-yl)-2-methoxypyridin-3-yl)methyl)((5-oxopyrrolidin-2-yl)methyl)carbamate (0.24 g, 0.35 mmol), K₂CO₃ (0.15 g, 1.06 mmol), and Pd(dppf)₂Cl₂-DCM (0.03 g, 0.04 mmol). The flask was purged with nitrogen for 5 min, then 2 mL of dry dioxane was added, and the reaction was sparged with nitrogen for 5 min. The mixture was stirred at 90° C. overnight thermally. The mixture was filtered through CELITE® and the filter cake was washed with EtOAc (3×40 mL). The filtrate was concentrated under reduced pressure. The residue was purified by normal phase flash SiO₂ chromatography using a gradient (0-5% methanol DCM) to afford tert-butyl ((6-(3′-(2-(((tert-butoxycarbonyl)(((S)-5-oxopyrrolidin-2-yl)methyl)amino) methyl)-4-oxo-1,4-dihydropyrido[2,3-d]pyrimidin-6-yl)-2,2′-dichloro-[1,1′-biphenyl]-3-yl)-2-methoxypyridin-3-yl)methyl)(((S)-5-oxopyrrolidin-2-yl)methyl)carbamate (0.066 g, 20% yield). MS: m/z found 927 [M+H]⁺.

### 6-(2,2′-Dichloro-3′-(6-methoxy-5-(((((S)-5-oxopyrrolidin-2-yl)methyl)amino)methyl)pyridin-2-yl)-[1,1′-biphenyl]-3-yl)-2-(((((S)-5-oxopyrrolidin-2-yl)methyl)amino)methyl)pyrido[2,3-d]pyrimidin-4(1H)-one

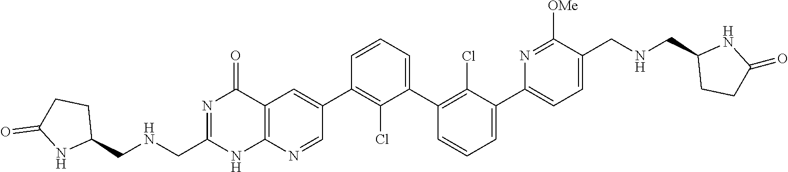 [SMILES: COc1nc(-c2cccc(-c3cccc(-c4cnc5[nH]c(CNC[C@@H]6CCC(=O)N6)nc(=O)c5c4)c3Cl)c2Cl)ccc1CNC[C@@H]1CCC(=O)N1]

A scintillation vial was charged with tert-butyl ((6-(3′-(2-(((tert-butoxycarbonyl)(((S)-5-oxopyrrolidin-2-yl)methyl)amino) methyl)-4-oxo-1,4-dihydropyrido[2,3-d]pyrimidin-6-yl)-2,2′-dichloro-[1,1′-biphenyl]-3-yl)-2-methoxypyridin-3-yl)methyl)(((S)-5-oxopyrrolidin-2-yl)methyl)carbamate (0.07 g, 0.08 mmol), then 5 ml of a 1:1 solution of TFA/DCM was added, and the reaction was stirred at room temp for 10 min. The reaction was concentrated, and residue purified by semi-prep HPLC (with 0.05% Formic acid modifier). ACN was evaporated off, and aqueous portion was lyophilized to give 5.6 mg (10%) of the product as bis formic acid salt affording 6-(2,2′-dichloro-3′-(6-methoxy-5-(((((S)-5-oxopyrrolidin-2-yl)methyl)amino)methyl)pyridin-2-yl)-[1,1′-biphenyl]-3-yl)-2-(((((S)-5-oxopyrrolidin-2-yl)methyl)amino)methyl)pyrido[2,3-d]pyrimidin-4(1H)-one as a white solid. MS: m/z found 727 [M+H]⁺. ¹H NMR (400 MHz, Methanol-d₄) δ 8.41 (s, 1H), 8.25 (d, J=2.1 Hz, 1H), 7.90 (dd, J=8.5, 2.1 Hz, 1H), 7.75 (t, J=8.3 Hz, 2H), 7.61 (dd, J=7.7, 1.7 Hz, 1H), 7.54-7.42 (m, 3H), 7.41-7.31 (m, 2H), 7.26 (d, J=7.5 Hz, 1H), 4.00 (d, J=13.2 Hz, 5H), 3.98-3.87 (m, 1H), 3.85 (s, 1H), 3.87-3.76 (m, 2H), 2.94-2.75 (m, 3H), 2.67 (dd, J=12.0, 7.5 Hz, 1H), 2.41-2.25 (m, 5H), 2.30-2.17 (m, 1H), 1.90-1.75 (m, 2H).

### Example 422: (S)-5-((((6-(2,2′-Dichloro-3′-(2-(((((S)-5-oxopyrrolidin-2-yl)methyl)amino)methyl)quinazolin-6-yl)-[1,1′-biphenyl]-3-yl)-2-methoxypyridin-3-yl)methyl)amino)methyl)pyrrolidin-2-one

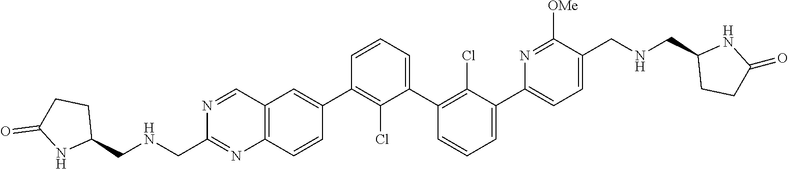 [SMILES: COc1nc(-c2cccc(-c3cccc(-c4ccc5nc(CNC[C@@H]6CCC(=O)N6)ncc5c4)c3Cl)c2Cl)ccc1CNC[C@@H]1CCC(=O)N1]

This compound was prepared in a similar fashion to Example 433, using Suzuki condition A, and using 6-bromo-2-(chloromethyl)quinazoline and (S)-5-(aminomethyl)pyrrolidin-2-one in the first step. MS: m/z found 710 [M+H]⁺; ¹H NMR (400 MHz, Methanol-d₄) δ 9.58 (s, 1H), 8.26 (s, 1H), 8.20 (d, J=1.9 Hz, 1H), 8.18-8.05 (m, 2H), 7.78 (d, J=7.5 Hz, 1H), 7.61 (d, J=7.7, 1.7 Hz, 1H), 7.58-7.43 (m, 3H), 7.43-7.34 (m, 2H), 7.28 (d, J=7.5 Hz, 1H), 4.46 (d, 2H), 4.10 (d, J=3.3 Hz, 2H), 4.04-3.99 (m, 3H), 4.03-3.89 (m, 2H), 3.18-2.92 (m, 4H), 2.43-2.25 (m, 6H), 1.98-1.77 (m, 2H).

### Example 421: 7-(2,2′-Dichloro-3′-(6-methoxy-5-(((((S)-5-oxopyrrolidin-2-yl)methyl)amino)methyl)pyridin-2-yl)-[1,1′-biphenyl]-3-yl)-2-(((((S)-5-oxopyrrolidin-2-yl)methyl)amino)methyl)quinazolin-4(1H)-one

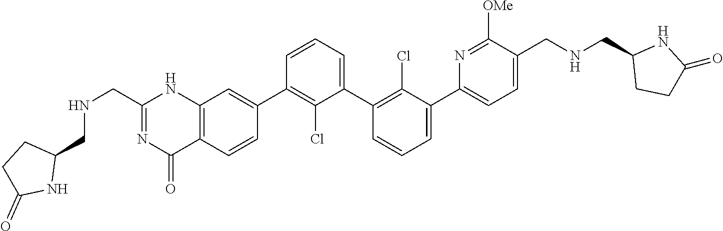 [SMILES: COc1nc(-c2cccc(-c3cccc(-c4ccc5c(=O)nc(CNC[C@@H]6CCC(=O)N6)[nH]c5c4)c3Cl)c2Cl)ccc1CNC[C@@H]1CCC(=O)N1]

This compound was prepared in a similar fashion to Example 433, using Suzuki condition A, and using 7-bromo-2-(chloromethyl)quinazolin-4(1H)-one and (S)-5-(aminomethyl)pyrrolidin-2-one in the first step. MS: m/z found 726 [M+H]⁺; ¹H NMR (400 MHz, Methanol-d₄) δ 8.41 (s, 1H), 8.22 (d, J=8.3 Hz, 1H), 7.75-7.68 (m, 2H), 7.58 (t, J=9.8, 8.0, 1.7 Hz, 2H), 7.53-7.41 (m, 3H), 7.36 (t, J=7.2, 2.1 Hz, 2H), 7.23 (d, J=7.5 Hz, 1H), 3.99 (s, 3H), 3.93-3.82 (m, 2H), 3.86-3.72 (m, 4H), 2.85-2.71 (m, 3H), 2.69-2.59 (m, 1H), 2.38-2.14 (m, 6H), 1.86-1.72 (m, 2H).

### Example 420: (S)-5-((((6-(2,2′-Dichloro-3′-(2-methyl-3-(((((S)-5-oxopyrrolidin-2-yl)methyl)amino)methyl)pyrazolo[1,5-a]pyrimidin-6-yl)-[1,1′-biphenyl]-3-yl)-2-methoxypyridin-3-yl)methyl)amino)methyl)pyrrolidin-2-one

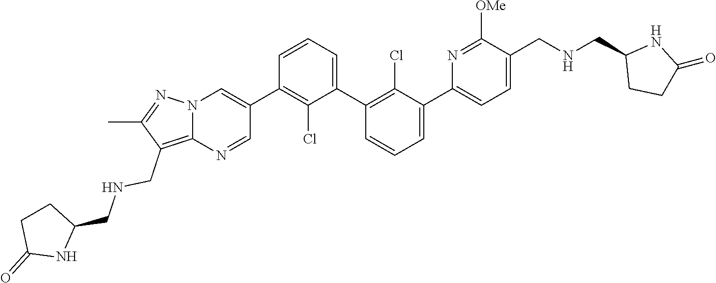 [SMILES: COc1nc(-c2cccc(-c3cccc(-c4cnc5c(CNC[C@@H]6CCC(=O)N6)c(C)nn5c4)c3Cl)c2Cl)ccc1CNC[C@@H]1CCC(=O)N1]

This compound was prepared in a similar fashion to Example 459, using Suzuki condition A, using 6-bromo-2-methylpyrazolo[1,5-a]pyrimidine-3-carbaldehyde and 6-(2,2′-dichloro-3′-(4,4,5,5-tetramethyl-1,3,2-dioxaborolan-2-yl)-[1,1′-biphenyl]-3-yl)-2-methoxynicotinaldehyde. MS: m/z found 713 [M+H]⁺; ¹H NMR (400 MHz, Methanol-d₄) δ 8.99 (d, J=2.1 Hz, 1H), 8.69 (d, J=2.1 Hz, 1H), 8.36 (s, 2H), 7.79 (d, J=7.5 Hz, 1H), 7.70-7.40 (m, 5H), 7.36 (d, J=7.6, 1.8 Hz, 1H), 7.26 (d, J=7.5 Hz, 1H), 4.38 (s, 2H), 4.08 (d, J=2.8 Hz, 2H), 4.04-3.88 (m, 5H), 3.19-3.05 (m, 2H), 3.05-2.90 (m, 2H), 2.54 (s, 3H), 2.40-2.21 (m, 6H), 1.91-1.74 (m, 2H).

### Example 397: (S)-5-((((6-(2,2′-Dichloro-3′-(4-methoxy-3-(((((S)-5-oxopyrrolidin-2-yl)methyl)amino)methyl)pyrazolo[1,5-a]pyridin-6-yl)-[1,1′-biphenyl]-3-yl)-2-methoxypyridin-3-yl)methyl)amino)methyl)pyrrolidin-2-one

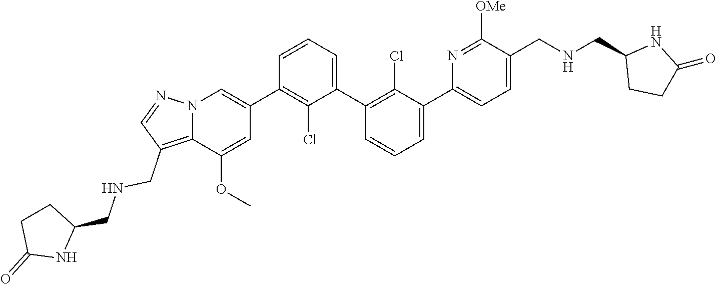 [SMILES: COc1nc(-c2cccc(-c3cccc(-c4cc(OC)c5c(CNC[C@@H]6CCC(=O)N6)cnn5c4)c3Cl)c2Cl)ccc1CNC[C@@H]1CCC(=O)N1]

This compound was prepared in a similar fashion to Example 459, using Suzuki condition A, using 6-bromo-4-methoxypyrazolo[1,5-a]pyridine-3-carbaldehyde and 6-(2,2′-dichloro-3′-(4,4,5,5-tetramethyl-1,3,2-dioxaborolan-2-yl)-[1,1′-biphenyl]-3-yl)-2-methoxynicotinaldehyde. MS: m/z found 728 [M+H]⁺; ¹H NMR (400 MHz, Methanol-d₄) δ 8.38 (s, 2H), 8.26 (s, 1H), 8.02 (s, 1H), 7.76 (d, J=7.6, 1.8 Hz, 1H), 7.60 (d, J=7.7, 1.7 Hz, 1H), 7.56-7.42 (m, 3H), 7.46-7.31 (m, 2H), 7.25 (d, J=7.5 Hz, 1H), 6.80 (s, 1H), 4.48 (s, 2H), 4.02 (d, J=9.4 Hz, 8H), 3.99-3.88 (m, 2H), 3.16-3.03 (m, 2H), 2.95-2.85 (m, 2H), 2.35-2.26 (m, 6H), 1.92-1.75 (m, 2H).

### Example 380: ((S)-5-((((6-(2,2′-Dichloro-3′-(3-(((((S)-5-oxopyrrolidin-2-yl)methyl)amino)methyl)imidazo[1,2-a]pyridin-7-yl)-[1,1′-biphenyl]-3-yl)-2-methoxypyridin-3-yl)methyl)amino)methyl)pyrrolidin-2-one

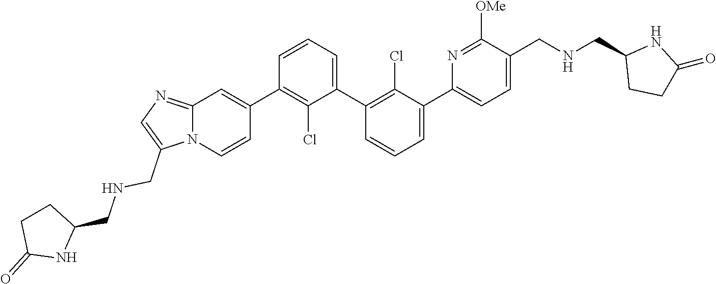 [SMILES: COc1nc(-c2cccc(-c3cccc(-c4ccn5c(CNC[C@@H]6CCC(=O)N6)cnc5c4)c3Cl)c2Cl)ccc1CNC[C@@H]1CCC(=O)N1]

This compound was prepared in a similar fashion to Example 459, using Suzuki condition A, using 7-bromoimidazo[1,2-a]pyridine-3-carbaldehyde and 6-(2,2′-dichloro-3′-(4,4,5,5-tetramethyl-1,3,2-dioxaborolan-2-yl)-[1,1′-biphenyl]-3-yl)-2-methoxynicotinaldehyde. MS: m/z found 698 [M+H]⁺; ¹H NMR (400 MHz, Methanol-d₄) δ 8.56 (d, J=7.1 Hz, 1H), 8.32 (s, 2H), 7.80 (d, J=7.5 Hz, 1H), 7.65-7.57 (m, 3H), 7.56-7.43 (m, 3H), 7.43-7.34 (m, 2H), 7.29 (d, J=7.5 Hz, 1H), 7.13 (d, J=7.1, 1.4 Hz, 1H), 4.22 (d, 2H), 4.12 (d, J=2.4 Hz, 2H), 4.06-3.90 (m, 4H), 3.78 (t, 1H), 3.09-2.95 (m, 2H), 2.83-2.67 (m, 2H), 2.42-2.22 (m, 5H), 2.26-2.13 (m, 1H), 1.92-1.70 (m, 2H).

### Example 379: (S)-5-((((6-(2,2′-Dichloro-3′-(2-methoxy-3-(((((S)-5-oxopyrrolidin-2-yl)methyl)amino)methyl)pyrazolo[1,5-a]pyrimidin-6-yl)-[1,1′-biphenyl]-3-yl)-2-methoxypyridin-3-yl)methyl)amino)methyl)pyrrolidin-2-one

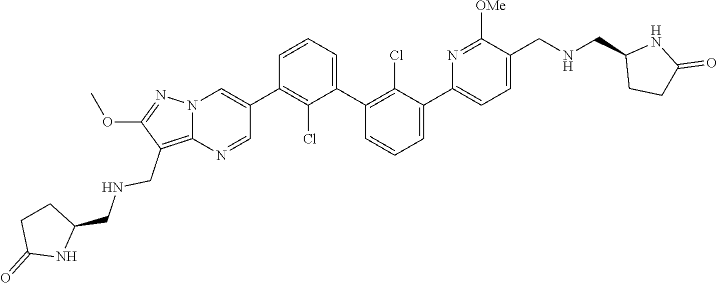 [SMILES: COc1nc(-c2cccc(-c3cccc(-c4cnc5c(CNC[C@@H]6CCC(=O)N6)c(OC)nn5c4)c3Cl)c2Cl)ccc1CNC[C@@H]1CCC(=O)N1]

This compound was prepared in a similar fashion to Example 459, using Suzuki condition A, using 6-bromo-2-methoxypyrazolo[1,5-a]pyrimidine-3-carbaldehyde and 6-(2,2′-dichloro-3′-(4,4,5,5-tetramethyl-1,3,2-dioxaborolan-2-yl)-[1,1′-biphenyl]-3-yl)-2-methoxynicotinaldehyde. MS: m/z found 729 [M+H]⁺; ¹H NMR (400 MHz, Methanol-d₄) δ 8.95 (s, 1H), 8.64 (s, 1H), 8.36 (s, 2H), 7.76 (d, 1H), 7.66-7.40 (m, 5H), 7.36 (d, J=7.6, 1.8, 0.9 Hz, 1H), 7.26 (d, J=7.4, 0.9 Hz, 1H), 4.28 (s, 2H), 4.11 (s, 3H), 4.01 (s, 5H), 3.96 (t, J=5.8 Hz, 1H), 3.89 (t, J=7.5 Hz, 1H), 3.17-3.03 (m, 2H), 2.96-2.81 (m, 2H), 2.41-2.22 (m, 6H), 1.91-1.75 (m, 2H).

### Example 311: (S)-5-((((6-(2,2′-Dichloro-3′-(2-(((((S)-5-oxopyrrolidin-2-yl)methyl)amino)methyl)furo[3,2-b]pyridin-6-yl)-[1,1′-biphenyl]-3-yl)-2-methoxypyridin-3-yl)methyl)amino)methyl)pyrrolidin-2-one

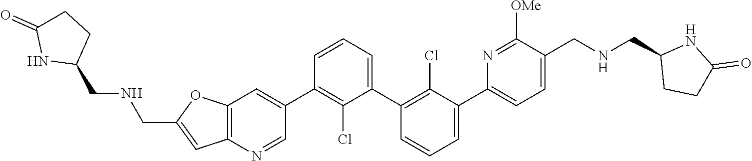 [SMILES: COc1nc(-c2cccc(-c3cccc(-c4cnc5cc(CNC[C@@H]6CCC(=O)N6)oc5c4)c3Cl)c2Cl)ccc1CNC[C@@H]1CCC(=O)N1]

This compound was prepared in a similar fashion to Example 459, using Suzuki condition A, using 6-bromofuro[3,2-b]pyridine-2-carbaldehyde and 6-(2,2′-dichloro-3′-(4,4,5,5-tetramethyl-1,3,2-dioxaborolan-2-yl)-[1,1′-biphenyl]-3-yl)-2-methoxynicotinaldehyde. MS: m/z found 699 [M+H]⁺; ¹H NMR (400 MHz, Methanol-d₄) δ 8.48 (d, J=1.8 Hz, 1H), 8.35 (s, 2H), 8.01 (d, J=2.0 Hz, 1H), 7.76 (d, J=7.5 Hz, 1H), 7.61 (d, J=7.7, 1.8 Hz, 1H), 7.53-7.42 (m, 3H), 7.37 (t, J=7.2, 3.1 Hz, 2H), 7.27 (d, J=7.5 Hz, 1H), 6.92 (s, 1H), 4.02 (d, J=3.5 Hz, 7H), 3.90 (s, 1H), 3.79 (t, 1H), 2.92 (s, 2H), 2.80-2.64 (m, 2H), 2.35-2.22 (m, 6H), 1.85-1.75 (m, 2H).

### Example 310: (S)-5-((((6-(2,2′-Dichloro-3′-(3-(((((S)-5-oxopyrrolidin-2-yl)methyl)amino)methyl)pyrazolo[1,5-a]pyrimidin-6-yl)-[1,1′-biphenyl]-3-yl)-2-methoxypyridin-3-yl)methyl)amino)methyl)pyrrolidin-2-one

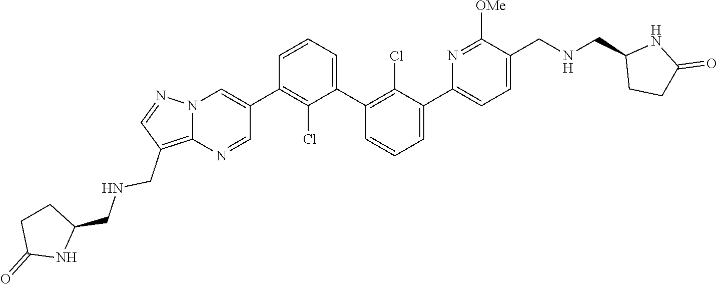 [SMILES: COc1nc(-c2cccc(-c3cccc(-c4cnc5c(CNC[C@@H]6CCC(=O)N6)cnn5c4)c3Cl)c2Cl)ccc1CNC[C@@H]1CCC(=O)N1]

This compound was prepared in a similar fashion to Example 459, using Suzuki condition A, using 6-bromopyrazolo[1,5-a]pyrimidine-3-carbaldehyde and 6-(2,2′-dichloro-3′-(4,4,5,5-tetramethyl-1,3,2-dioxaborolan-2-yl)-[1,1′-biphenyl]-3-yl)-2-methoxynicotinaldehyde. MS: m/z found 699 [M+H]⁺; ¹H NMR (400 MHz, Methanol-d₄) δ 9.08 (s, 1H), 8.71 (s, 1H), 8.42 (s, 2H), 8.28 (s, 1H), 7.73 (d, J=7.4 Hz, 1H), 7.65-7.56 (m, 2H), 7.55 (t, J=7.6, 1.0 Hz, 1H), 7.52-7.41 (m, 2H), 7.36 (d, J=7.6, 1.8, 0.9 Hz, 1H), 7.23 (d, J=7.4, 1.1 Hz, 1H), 4.60 (s, 1H), 4.29 (s, 2H), 3.99 (s, 3H), 3.95-3.83 (m, 3H), 3.03-2.91 (m, 2H), 2.85-2.72 (m, 2H), 2.39-2.21 (m, 6H), 1.88-1.73 (m, 2H).

### Example 309: (S)-5-((((6-(2,2′-Dichloro-3′-(3-(((((S)-5-oxopyrrolidin-2-yl)methyl)amino)methyl)imidazo[1,2-a]pyrazin-6-yl)-[1,1′-biphenyl]-3-yl)-2-methoxypyridin-3-yl)methyl)amino)methyl)pyrrolidin-2-one

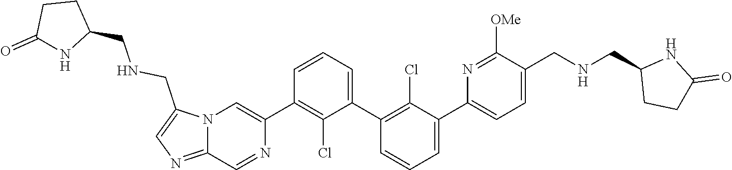 [SMILES: COc1nc(-c2cccc(-c3cccc(-c4cn5c(CNC[C@@H]6CCC(=O)N6)cnc5cn4)c3Cl)c2Cl)ccc1CNC[C@@H]1CCC(=O)N1]

This compound was prepared in a similar fashion to Example 459, using Suzuki condition A, using 6-bromoimidazo[1,2-a]pyrazine-3-carbaldehyde and 6-(2,2′-dichloro-3′-(4,4,5,5-tetramethyl-1,3,2-dioxaborolan-2-yl)-[1,1′-biphenyl]-3-yl)-2-methoxynicotinaldehyde. MS: m/z found 699 [M+H]⁺; ¹H NMR (400 MHz, Methanol-d₄) δ 9.04 (d, J=1.5 Hz, 1H), 8.81 (d, 1H), 8.28 (s, 2H), 7.79 (s, 3H), 7.69 (d, J=7.6 Hz, 1H), 7.61 (s, 1H), 7.57-7.40 (m, 2H), 7.38 (d, J=7.1 Hz, 1H), 7.28 (s, 1H), 4.21 (s, 2H), 4.11 (s, 2H), 4.03 (s, 2H), 3.94 (s, 2H), 3.71 (s, 1H), 3.01 (s, 2H), 2.66 (d, 2H), 2.33 (s, 3H), 2.17-2.16 (m, 3H), 1.88-1.81 (m, 1H), 1.75 (s, 1H).

### Example 248: (S)-5-((((6-(2,2′-Dichloro-3′-(2-(((((S)-5-oxopyrrolidin-2-yl)methyl) amino)methyl)benzo[d]oxazol-6-yl)-[1,1′-biphenyl]-3-yl)-2-methoxypyridin-3-yl)methyl)amino)methyl)pyrrolidin-2-one

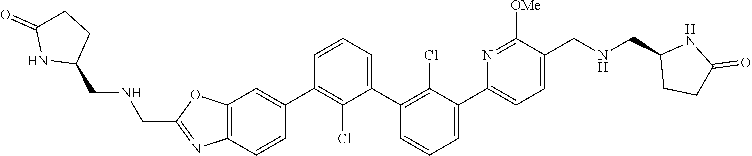 [SMILES: COc1nc(-c2cccc(-c3cccc(-c4ccc5nc(CNC[C@@H]6CCC(=O)N6)oc5c4)c3Cl)c2Cl)ccc1CNC[C@@H]1CCC(=O)N1]

This compound was prepared in a similar fashion to Example 459, using Suzuki condition A, using 6-bromobenzo[d]oxazole-2-carbaldehyde and 6-(2,2′-dichloro-3′-(4,4,5,5-tetramethyl-1,3,2-dioxaborolan-2-yl)-[1,1′-biphenyl]-3-yl)-2-methoxynicotinaldehyde. MS: m/z found 699 [M+H]⁺; ¹H NMR (400 MHz, Methanol-d₄) δ 8.38 (s, 1H), 7.78-7.64 (m, 3H), 7.60 (d, J=7.7, 1.8 Hz, 1H), 7.50-7.39 (m, 4H), 7.39-7.29 (m, 2H), 7.25 (d, 1H), 4.10 (s, 2H), 3.99 (d, J=15.1 Hz, 5H), 3.87 (t, 1H), 3.85-3.75 (m, 1H), 2.90-2.76 (m, 3H), 2.71 (dd, J=12.0, 7.2 Hz, 1H), 2.40-2.25 (m, 6H), 2.28-2.15 (m, 1H), 1.86-1.77 (m, 1H).

### Example 247: (S)-5-((((6-(2,2′-Dichloro-3′-(2-(((((S)-5-oxopyrrolidin-2-yl)methyl) amino)methyl)benzo[d]oxazol-5-yl)-[1,1′-biphenyl]-3-yl)-2-methoxypyridin-3-yl)methyl)amino)methyl)pyrrolidin-2-one

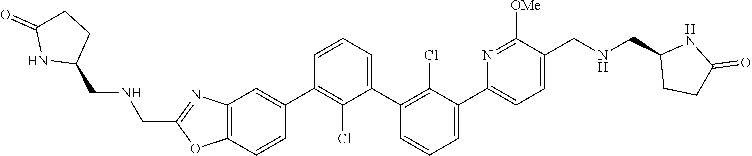 [SMILES: COc1nc(-c2cccc(-c3cccc(-c4ccc5oc(CNC[C@@H]6CCC(=O)N6)nc5c4)c3Cl)c2Cl)ccc1CNC[C@@H]1CCC(=O)N1]

This compound was prepared in a similar fashion to Example 459, using Suzuki condition A, using 5-bromobenzo[d]oxazole-2-carbaldehyde and 6-(2,2′-dichloro-3′-(4,4,5,5-tetramethyl-1,3,2-dioxaborolan-2-yl)-[1,1′-biphenyl]-3-yl)-2-methoxynicotinaldehyde. MS: m/z found 699 [M+H]⁺; ¹H NMR (400 MHz, Methanol-d₄) δ 8.42 (s, 1H), 7.76-7.68 (m, 2H), 7.68-7.55 (m, 2H), 7.50-7.40 (m, 4H), 7.39-7.29 (m, 2H), 7.24 (d, J=7.4 Hz, 1H), 4.11 (d, J=1.5 Hz, 2H), 3.99 (d, J=2.3 Hz, 3H), 3.93-3.75 (m, 4H), 2.85-2.66 (m, 4H), 2.36-2.17 (m, 6H), 1.86-1.76 (m, 2H).

### Example 239: (S)-6-(2,2′-Dichloro-3′-(6-methoxy-5-((((5-oxopyrrolidin-2-yl)methyl)amino)methyl)pyridin-2-yl)-[1,1′-biphenyl]-3-yl)-2-(2-hydroxyethyl)-[1,2,4]triazolo[4,3-a]pyridin-3(2H)-one

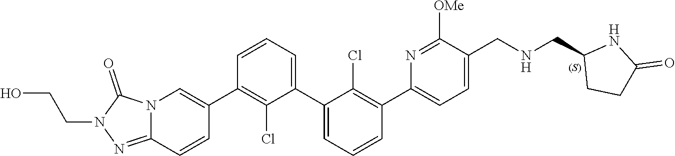

### 6-Bromo-2-(2-hydroxyethyl)-[1,2,4]triazolo[4,3-a]pyridin-3(2H)-one

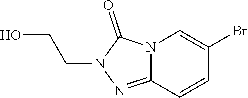 [SMILES: O=c1n(CCO)nc2ccc(Br)cn12]

To a stirring solution of 6-bromo-2H-[1,2,4]triazolo[4,3-a]pyridin-3-one (200.00 mg, 0.93 mmol) at rt in dry DMF (2 mL) under N₂ was added potassium phosphate (347 mg, 1.64 mmol) in one portion. After 15 min stirring at rt, 2-bromoethanol (0.10 mL, 1.40 mmol) was added dropwise via syringe. The reaction mixture was stirred at rt for 24 h. The reaction was filtered, washed with minimal EtOAc, evaporated, dissolved in DCM, and washed with water (x2). The organic phase was dried over anhydrous sodium sulfate, filtered, and evaporated to dryness. The crude oil was purified by flash column chromatography 0-10% MeOH/DCM to afford 6-bromo-2-(2-hydroxyethyl)-[1,2,4]triazolo[4,3-a]pyridin-3(2H)-one (124 mg, 51% yield), m/z: 258 [M]+ observed. ¹H NMR (400 MHz, DMSO-d₆) δ 8.17-8.10 (m, 1H), 7.32-7.22 (m, 2H), 4.80 (t, J=5.8 Hz, 1H), 3.91 (t, J=5.8 Hz, 2H), 3.70 (t, J=6.3 Hz, 2H).

### tert-Butyl (S)-((6-(2,2′-dichloro-3′-(2-(2-hydroxyethyl)-3-oxo-2,3-dihydro-[1,2,4]triazolo[4,3-a]pyridin-6-yl)-[1,1′-biphenyl]-3-yl)-2-methoxypyridin-3-yl)methyl)((5-oxopyrrolidin-2-yl)methyl)carbamate

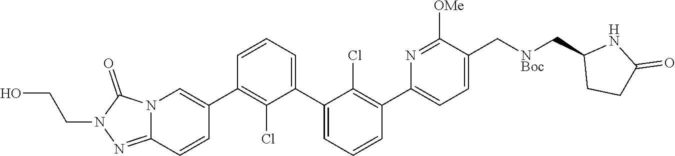 [SMILES: COc1nc(-c2cccc(-c3cccc(-c4ccc5nn(CCO)c(=O)n5c4)c3Cl)c2Cl)ccc1CN(C[C@@H]1CCC(=O)N1)C(=O)OC(C)(C)C]

To a reaction vial equipped with stir bar and charged with tert-butyl N-[[6-[2-chloro-3-[2-chloro-3-(4,4,5,5-tetramethyl-1,3,2-dioxaborolan-2-yl)phenyl]phenyl]-2-methoxy-3-pyridyl]methyl]-N-[[(2S)-5-oxopyrrolidin-2-yl]methyl]carbamate (62 mg, 0.09 mmol), 6-bromo-2-(2-hydroxyethyl)-[1,2,4]triazolo[4,3-a]pyridin-3-one (28 mg, 0.11 mmol), potassium phosphate, (57 mg, 0.27 mmol), and PdCl₂(dtbpf) (6 mg, 0.01 mmol) was added a 5:1 mixture of dioxane/water (3 mL). The red suspension was sparged with N₂ for 5 minutes before moving to a heating block at 90° C. for 1 h. The reaction was cooled and quenched by the addition of saturated aqueous NaHCO₃ solution. The aqueous was extracted with EtOAc (3×5 mL), combined organic phases were dried over anhydrous sodium sulfate, filtered, and concentrated. The crude material was purified by flash silica column chromatography 0-10% MeOH/DCM to afford tert-butyl (S)-((6-(2,2′-dichloro-3′-(2-(2-hydroxyethyl)-3-oxo-2,3-dihydro-[1,2,4]triazolo[4,3-a]pyridin-6-yl)-[1,1′-biphenyl]-3-yl)-2-methoxypyridin-3-yl)methyl)((5-oxopyrrolidin-2-yl)methyl)carbamate (51 mg, 77% yield, m/z: 733 [M]⁺ observed).

### (S)-6-(2,2′-Dichloro-3′-(6-methoxy-5-((((5-oxopyrrolidin-2-yl)methyl)amino)methyl)pyridin-2-yl)-[1,1′-biphenyl]-3-yl)-2-(2-hydroxyethyl)-[1,2,4]triazolo[4,3-a]pyridin-3(2H)-one

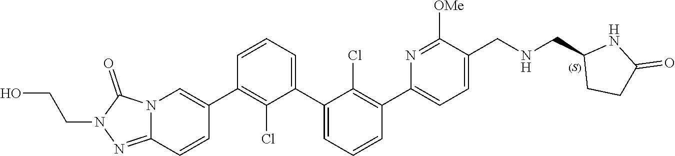

To a stirring solution of tert-butyl N-[[6-[2-chloro-3-[2-chloro-3-[2-(2-hydroxyethyl)-3-oxo-[1,2,4]triazolo[4,3-a]pyridin-6-yl]phenyl]phenyl]-2-methoxy-3-pyridyl]methyl]-N-[[(2S)-5-oxopyrrolidin-2-yl]methyl]carbamate (51 mg, 0.07 mmol) in dry dioxane (3 mL) and MeOH (100 μL) was added a 4M dioxane solution of hydrogen chloride (0.43 mL, 1.74 mmol) in a steady stream at rt. After 2.5h the reaction was complete by LCMS analysis. The solvent and excess acid were removed under vacuum and the crude material was purified by reverse phase HPLC to afford (S)-6-(2,2′-dichloro-3′-(6-methoxy-5-((((5-oxopyrrolidin-2-yl)methyl)amino)methyl)pyridin-2-yl)-[1,1′-biphenyl]-3-yl)-2-(2-hydroxyethyl)-[1,2,4]triazolo[4,3-a]pyridin-3(2H)-one as a white solid (10 mg). m/z: 622.4 [M]⁺ observed. ¹H NMR (400 MHz, Methanol-d₄) δ 8.42 (s, 2H), 7.90 (d, J=1.5 Hz, 1H), 7.83 (d, J=8.4 Hz, 1H), 7.65 (dd, J=8.8, 2.6 Hz, 1H), 7.53 (td, J=7.5, 6.3, 3.6 Hz, 3H), 7.42 (ddd, J=10.0, 7.5, 5.0 Hz, 3H), 7.38-7.25 (m, 2H), 4.16-4.06 (m, 6H), 3.96 (d, J=7.7 Hz, 2H), 3.01 (d, J=5.8 Hz, 2H), 2.45-2.31 (m, 3H), 1.88 (s, 1H), 1.30 (s, 2H).

### Example 240: (S)-5-((((6-(2,2′-Dichloro-3′-(5,6,7,8-tetrahydro-[1,2,4]triazolo[4,3-a]pyrazin-3-yl)-[1,1′-biphenyl]-3-yl)-2-methoxypyridin-3-yl)methyl)amino)methyl)pyrrolidin-2-one

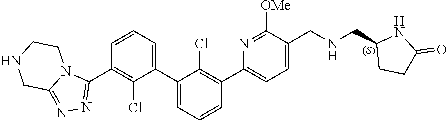

### tert-butyl (S)-3-(3′-(5-(((tert-butoxycarbonyl)((5-oxopyrrolidin-2-yl)methyl)amino)methyl)-6-methoxypyridin-2-yl)-2,2′-dichloro-[1,1′-biphenyl]-3-yl)-5,6-dihydro-[1,2,4]triazolo[4,3-a]pyrazine-7(8H)-carboxylate

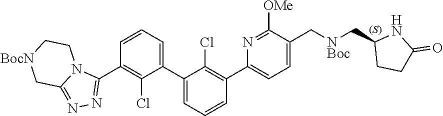

To a 20 mL reaction vial equipped with stirbar and charged with tert-butyl N-[[6-[2-chloro-3-[2-chloro-3-(4,4,5,5-tetramethyl-1,3,2-dioxaborolan-2-yl)phenyl]phenyl]-2-methoxy-3-pyridyl]methyl]-N-[[(2S)-5-oxopyrrolidin-2-yl]methyl]carbamate (62 mg, 0.09 mmol), tert-butyl 3-bromo-6,8-dihydro-5H-[1,2,4]triazolo[4,3-a]pyrazine-7-carboxylate (33 mg, 0.11 mmol), potassium phosphate (58 mg, 0.27 mmol), and PdCl₂(dtbpf) (6 mg, 0.01 mmol) was added a 5:1 mixture of dioxane/water (3 mL). The red suspension was sparged with N₂ for 5 min, moved to heating block at 90° C., and heated while stirring for 1 h. The reaction was cooled to rt and quenched by the addition of saturated aqueous NaHCO₃ solution. The aqueous was extracted with EtOAc (3×5 mL). Combined organic phases were dried over anhydrous sodium sulfate, filtered and concentrated. The crude material was purified by flash silica column chromatography 0-10% MeOH/DCM to afford tert-butyl (S)-3-(3′-(5-(((tert-butoxycarbonyl)((5-oxopyrrolidin-2-yl)methyl)amino)methyl)-6-methoxypyridin-2-yl)-2,2′-dichloro-[1,1′-biphenyl]-3-yl)-5,6-dihydro-[1,2,4]triazolo[4,3-a]pyrazine-7(8H)-carboxylate (27 mg, 29% yield, m/z: 778 [M]+ observed).

### (S)-5-((((6-(2,2′-Dichloro-3′-(5,6,7,8-tetrahydro-[1,2,4]triazolo[4,3-a]pyrazin-3-yl)-[1,1′-biphenyl]-3-yl)-2-methoxypyridin-3-yl)methyl)amino)methyl)pyrrolidin-2-one

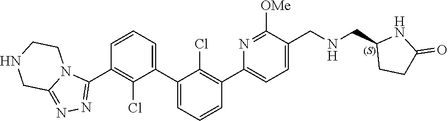

To a stirring solution of tert-butyl 3-[3-[3-[5-[[tert-butoxycarbonyl-[[(2S)-5-oxopyrrolidin-2-yl]methyl]amino]methyl]-6-methoxy-2-pyridyl]-2-chloro-phenyl]-2-chloro-phenyl]-6,8-dihydro-5H-[1,2,4]triazolo[4,3-a]pyrazine-7-carboxylate (27 mg, 0.03 mmol) in dioxane (2 mL) and MeOH (100 μL) at rt was added a 4 M dioxane solution of hydrogen chloride (0.22 mL, 0.87 mmol) in a steady stream. After 2.5 h the reaction was complete by LCMS analysis. The solvent and excess acid were removed under vacuum and the crude material was purified by reverse phase HPLC to afford (S)-5-((((6-(2,2′-dichloro-3′-(5,6,7,8-tetrahydro-[1,2,4]triazolo[4,3-a]pyrazin-3-yl)-[1,1′-biphenyl]-3-yl)-2-methoxypyridin-3-yl)methyl)amino)methyl)pyrrolidin-2-one as a white solid (6.0 mg). m/z: 578.3 [M]⁺ observed. ¹H NMR (400 MHz, Methanol-d₄) δ 8.42 (s, 2H), 8.29 (d, J=14.6 Hz, 1H), 7.83 (d, J=8.6 Hz, 1H), 7.68-7.62 (m, 3H), 7.53 (dd, J=8.8, 6.7 Hz, 1H), 7.42 (d, J=7.9 Hz, 1H), 7.31 (d, J=8.3 Hz, 1H), 4.26-3.91 (m, 11H), 3.22 (d, J=5.8 Hz, 1H), 3.02 (s, 2H), 2.43-2.34 (m, 3H), 1.88 (d, J=8.8 Hz, 1H), 1.30 (s, 1H).

### Example 241: (S)-5-((((6-(2,2′-Dichloro-3′-(2-(4-(2-hydroxyethyl)piperazin-1-yl)imidazo[2,1-b][1,3,4]thiadiazol-5-yl)-[1,1′-biphenyl]-3-yl)-2-methoxypyridin-3-yl)methyl)amino)methyl)pyrrolidin-2-one

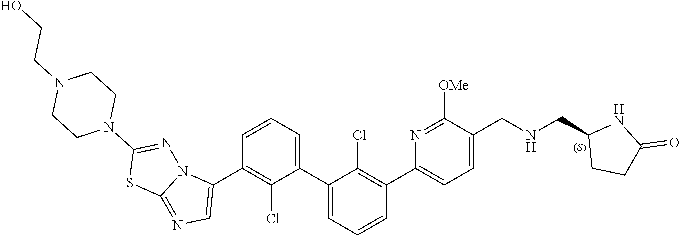

### 2-(4-(5-Iodoimidazo[2,1-b][1,3,4]thiadiazol-2-yl)piperazin-1-yl)ethan-1-ol

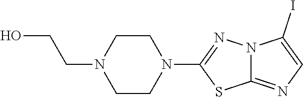 [SMILES: OCCN1CCN(c2nn3c(I)cnc3s2)CC1]

To a stirring suspension of 2-bromo-5-iodo-imidazo[2,1-b][1,3,4]thiadiazole (171 mg, 0.52 mmol) in MeCN (10 mL, 0.05 M) at rt was added N,N-diethylethanamine (0.22 mL, 1.55 mmol) dropwise via syringe, followed by 2-piperazin-1-ylethanol (101 mg, 0.78 mmol). The suspension was heated to 90° C. in a sealed reaction vial while stirring vigorously for 16 h. The reaction mixture filtered and concentrated to afford 2-(4-(5-iodoimidazo[2,1-b][1,3,4]thiadiazol-2-yl)piperazin-1-yl)ethan-1-ol (110 mg, 56% yield, m/z: 380 [M+H]⁺ observed). ¹H NMR (400 MHz, Methanol-d₄) δ 7.10 (s, 1H), 3.79 (t, J=5.5 Hz, 2H), 3.69-3.60 (m, 4H), 3.05-2.96 (m, 4H), 2.88 (t, J=5.5 Hz, 2H).

### tert-Butyl (S)-((6-(2,2′-dichloro-3′-(2-(4-(2-hydroxyethyl)piperazin-1-yl)imidazo[2,1-b][1,3,4]thiadiazol-5-yl)-[1,1′-biphenyl]-3-yl)-2-methoxypyridin-3-yl)methyl)((5-oxopyrrolidin-2-yl)methyl)carbamate

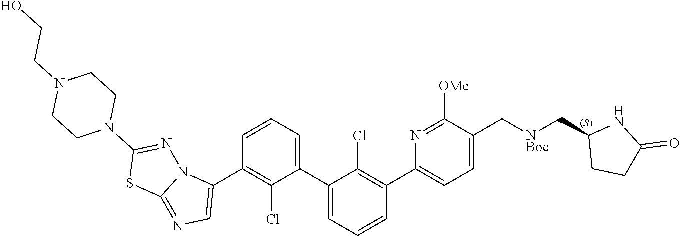

To a reaction vial equipped with stir bar and charged with tert-butyl N-[[6-[2-chloro-3-[2-chloro-3-(4,4,5,5-tetramethyl-1,3,2-dioxaborolan-2-yl)phenyl]phenyl]-2-methoxy-3-pyridyl]methyl]-N-[[(2S)-5-oxopyrrolidin-2-yl]methyl]carbamate (62 mg, 0.09 mmol), 2-[4-(5-iodoimidazo[2,1-b][1,3,4]thiadiazol-2-yl)piperazin-1-yl]ethanol (41 mg, 0.11 mmol), potassium phosphate (57 mg, 0.27 mmol), and PdCl₂(dtbpf) (6 mg, 0.01 mmol) was added a 5:1 mixture of dioxane/water (4 mL). The red suspension was sparged with N₂ for 5 minutes, then heated to 90° C. for 1 h. The reaction was cooled to rt and quenched by the addition of saturated aqueous NaHCO₃ solution. The aqueous was extracted with DCM (3×5 mL). Combined organic phases were dried over anhydrous sodium sulfate, filtered, and concentrated. The crude material was purified by flash silica column chromatography 0-10% MeOH/DCM to afford tert-butyl (S)-((6-(2,2′-dichloro-3′-(2-(4-(2-hydroxyethyl)piperazin-1-yl)imidazo[2,1-b][1,3,4]thiadiazol-5-yl)-[1,1′-biphenyl]-3-yl)-2-methoxypyridin-3-yl)methyl)((5-oxopyrrolidin-2-yl)methyl)carbamate (27 mg, 36% yield, m/z: 807 [M+H]⁺ observed).

### (S)-5-((((6-(2,2′-Dichloro-3′-(2-(4-(2-hydroxyethyl)piperazin-1-yl)imidazo[2,1-b][1,3,4]thiadiazol-5-yl)-[1,1′-biphenyl]-3-yl)-2-methoxypyridin-3-yl)methyl)amino)methyl)pyrrolidin-2-one

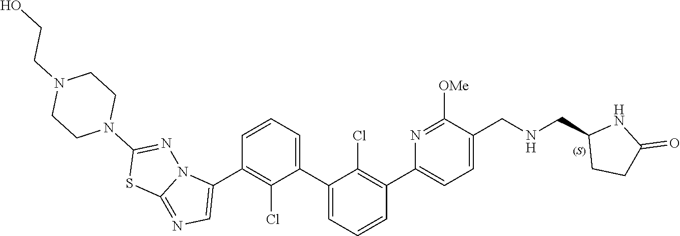

To a stirring solution of tert-butyl N-[[6-[2-chloro-3-[2-chloro-3-[2-[4-(2-hydroxyethyl)piperazin-1-yl]imidazo[2,1-b][1,3,4]thiadiazol-5-yl]phenyl]phenyl]-2-methoxy-3-pyridyl]methyl]-N-[[(2S)-5-oxopyrrolidin-2-yl]methyl]carbamate (27 mg, 0.03 mmol) in dioxane (2 mL) at rt was added a 4M dioxane solution of hydrogen chloride (0.21 mL, 0.84 mmol) in a steady stream. The reaction was monitored by LCMS and complete after 2 h. The solvent and excess acid were removed under vacuum and the crude material was purified by reverse phase HPLC to afford (S)-6-(2,2′-dichloro-3′-(6-methoxy-5-((((5-oxopyrrolidin-2-yl)methyl)amino)methyl)pyridin-2-yl)-[1,1′-biphenyl]-3-yl)-2-(3-(diisobutylamino)propyl)-[1,2,4]triazolo[4,3-a]pyridin-3(2H)-one (7.0 mg). m/z: 707 [M]⁺ observed. ¹H NMR (400 MHz, Chloroform-d) δ 8.40 (s, 1H), 7.91-7.80 (m, 2H), 7.64 (dd, J=8.4, 2.4 Hz, 1H), 7.50 (dd, J=9.1, 5.8 Hz, 2H), 7.46-7.28 (m, 4H), 4.20-4.04 (m, 5H), 3.97 (d, J=6.3 Hz, 1H), 3.79-3.69 (m, 2H), 3.56 (d, J=5.7 Hz, 4H), 3.00 (d, J=5.3 Hz, 2H), 2.73 (d, J=5.7 Hz, 4H), 2.70-2.61 (m, 3H), 2.47-2.34 (m, 3H), 1.98-1.82 (m, 1H).

### Example 242: (S)-5-((((6-(2,2′-Dichloro-3′-(7-(2-hydroxyethyl)-5,6,7,8-tetrahydro-[1,2,4]triazolo[4,3-a]pyrazin-3-yl)-[1,1′-biphenyl]-3-yl)-2-methoxypyridin-3-yl)methyl)amino)methyl)pyrrolidin-2-one

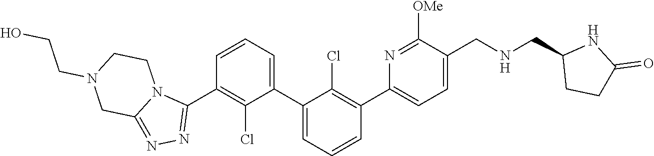 [SMILES: COc1nc(-c2cccc(-c3cccc(-c4nnc5n4CCN(CCO)C5)c3Cl)c2Cl)ccc1CNC[C@@H]1CCC(=O)N1]

### 2-(3-Bromo-5,6-dihydro-[1,2,4]triazolo[4,3-a]pyrazin-7(8H)-yl)ethan-1-ol

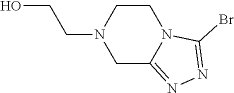 [SMILES: OCCN1CCn2c(Br)nnc2C1]

To a stirring solution of 3-bromo-5,6,7,8-tetrahydro-[1,2,4]triazolo[4,3-a]pyrazine (150 mg, 0.74 mmol) in DMF (2.5 mL) at rt under N₂ was added potassium carbonate (257 mg, 1.85 mmol) in one portion. The mixture was stirred at rt for 15 min before adding 2-bromoethanol (0.08 mL, 1.11 mmol) dropwise via syringe. The reaction mixture was stirred at rt overnight and then heated at 45° C. for 4 h. The reaction was filtered and the filtrate was reduced to afford 2-(3-bromo-5,6-dihydro-[1,2,4]triazolo[4,3-a]pyrazin-7(8H)-yl)ethan-1-ol (0.1 g, 55% yield, m/z: 247 [M]⁺ observed).

### tert-Butyl (S)-((6-(2,2′-dichloro-3′-(7-(2-hydroxyethyl)-5,6,7,8-tetrahydro-[1,2,4]triazolo[4,3-a]pyrazin-3-yl)-[1,1′-biphenyl]-3-yl)-2-methoxypyridin-3-yl)methyl)((5-oxopyrrolidin-2-yl)methyl)carbamate

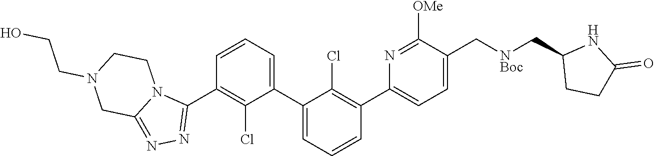 [SMILES: COc1nc(-c2cccc(-c3cccc(-c4nnc5n4CCN(CCO)C5)c3Cl)c2Cl)ccc1CN(C[C@@H]1CCC(=O)N1)C(=O)OC(C)(C)C]

To a 20 mL reaction vial equipped with stirbar and charged with tert-butyl N-[[6-[2-chloro-3-[2-chloro-3-(4,4,5,5-tetramethyl-1,3,2-dioxaborolan-2-yl)phenyl]phenyl]-2-methoxy-3-pyridyl]methyl]-N-[[(2S)-5-oxopyrrolidin-2-yl]methyl]carbamate (56 mg, 0.08 mmol), potassium carbonate (34 mg, 0.25 mmol), Pd(PPh₃)₄ (10 mg, 0.01 mmol), and 2-(3-bromo-6,8-dihydro-5H-[1,2,4]triazolo[4,3-a]pyrazin-7-yl)ethanol (26 mg, 0.11 mmol) was added a 5:1 mixture of dioxane/water (3 mL). The red suspension was sparged with N₂ for 5 min and then heated for 1 h at 95° C. The reaction upon completion was evaporated to dryness, dissolved in DCM and purified by flash silica column chromatography 0-10% MeOH/DCM to afford tert-butyl (S)-((6-(2,2′-dichloro-3′-(7-(2-hydroxyethyl)-5,6,7,8-tetrahydro-[1,2,4]triazolo[4,3-a]pyrazin-3-yl)-[1,1′-biphenyl]-3-yl)-2-methoxypyridin-3-yl)methyl)((5-oxopyrrolidin-2-yl)methyl)carbamate (22 mg, 37% yield, m/z: 722 [M]⁺ observed).

### (S)-5-((((6-(2,2′-Dichloro-3′-(7-(2-hydroxyethyl)-5,6,7,8-tetrahydro-[1,2,4]triazolo[4,3-a]pyrazin-3-yl)-[1,1′-biphenyl]-3-yl)-2-methoxypyridin-3-yl)methyl)amino)methyl)pyrrolidin-2-one

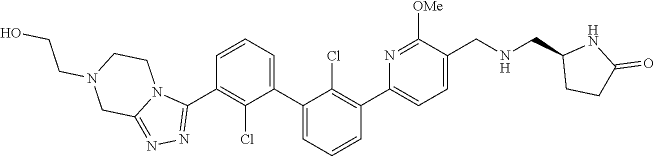 [SMILES: COc1nc(-c2cccc(-c3cccc(-c4nnc5n4CCN(CCO)C5)c3Cl)c2Cl)ccc1CNC[C@@H]1CCC(=O)N1]

To a stirring solution of tert-butyl N-[[6-[2-chloro-3-[2-chloro-3-[7-(2-hydroxyethyl)-6,8-dihydro-5H-[1,2,4]triazolo[4,3-a]pyrazin-3-yl]phenyl]phenyl]-2-methoxy-3-pyridyl]methyl]-N-[[(2S)-5-oxopyrrolidin-2-yl]methyl]carbamate (22 mg, 0.03 mmol) in dioxane (2 mL) at rt was added a 4M dioxane solution of hydrogen chloride (0.19 mL, 0.76 mmol) in a steady stream. The reaction was monitored by LCMS and after 2 h shows full conversion. The solvent and excess acid were removed under vacuum and the crude material was purified by reverse phase HPLC to afford (S)-5-((((6-(2,2′-dichloro-3′-(7-(2-hydroxyethyl)-5,6,7,8-tetrahydro-[1,2,4]triazolo[4,3-a]pyrazin-3-yl)-[1,1′-biphenyl]-3-yl)-2-methoxypyridin-3-yl)methyl)amino)methyl)pyrrolidin-2-one as a white solid (6.0 mg). m/z: 622.4 [M]⁺ observed. ¹H NMR (400 MHz, Methanol-d₄) δ 8.40 (s, 1H), 7.82 (d, J=8.1 Hz, 1H), 7.67 (dd, J=7.7, 1.8 Hz, 2H), 7.62 (s, 2H), 7.53 (dd, J=8.7, 6.5 Hz, 1H), 7.42 (d, J=7.4 Hz, 1H), 7.31 (d, J=8.6 Hz, 1H), 4.08 (d, J=18.3 Hz, 5H), 3.99 (d, J=10.7 Hz, 5H), 3.77 (q, J=6.3, 5.4 Hz, 2H), 3.08-2.97 (m, 4H), 2.87-2.79 (m, 2H), 2.42-2.34 (m, 3H), 1.88 (d, J=8.0 Hz, 1H), 1.30 (s, 1H).

### Example 312: (S)-5-((((6-(2,2′-Dichloro-3′-(1-methyl-2-(((((S)-5-oxopyrrolidin-2-yl)methyl)amino)methyl)-1H-imidazo[4,5-b]pyridin-5-yl)-[1,1′-biphenyl]-3-yl)-2-methoxypyridin-3-yl)methyl)amino)methyl)pyrrolidin-2-one

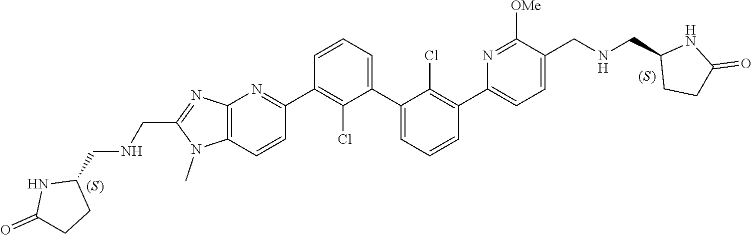

### 2,5-Dichloro-1-methyl-1H-imidazo[4,5-b]pyridine

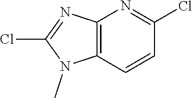

To a stirring solution of 2,5-dichloro-1H-imidazo[4,5-b]pyridine (300 mg, 1.60 mmol) in DMF (8 mL) at rt was added potassium carbonate (441 mg, 3.19 mmol) in one portion. The mixture was stirred for 10 min at rt under N₂ prior to the dropwise addition of iodomethane (0.12 mL, 1.91 mmol) via syringe. The reaction mixture was heated to 50° C. while stirring and after 1 h of heating the reaction was complete. The reaction was cooled to rt, filtered to remove the white solid. The reaction was diluted with water (20 mL) and EtOAc (20 mL) and the aqueous phase was extracted with EtOAc (2×25 mL). The combined organic phases were washed with water until DMF was removed, dried over anhydrous sodium sulfate, filtered, and concentrated to afford 2,5-dichloro-1-methyl-1H-imidazo[4,5-b]pyridine (220 mg, 68% yield, m/z: 202 [M]⁺ observed).

### 5-Chloro-1-methyl-2-vinyl-1H-imidazo[4,5-b]pyridine

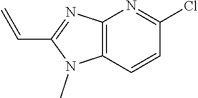

To a reaction vial charged with 2,5-dichloro-1-methyl-imidazo[4,5-b]pyridine (400 mg, 1.98 mmol), potassium carbonate (820 mg, 5.94 mmol), PdCl₂(dppf) (162 mg, 0.20 mmol), and potassium vinyltrifluoroborate (318 mg, 2.38 mmol) at rt under N₂ was added a 4:1 dioxane/water mixture (20 mL). The suspension was sparged with N₂ for 10 minutes prior to heating the mixture to 80° C. while stirring for 12 h. The reaction was cooled to rt, the water layer was removed and extracted with EA (2×5 mL), combined organic layers were filtered over a thin bed of Celite and anhydrous sodium sulfate and concentrated. The crude oil was purified by silica flash column chromatography 0-10% MeOH/DCM to afford 5-chloro-1-methyl-2-vinyl-1H-imidazo[4,5-b]pyridine (178 mg, 46% yield). m/z: 194 [M+H]⁺ observed. ¹H NMR (400 MHz, Chloroform-d) δ 8.11-8.03 (m, 1H), 7.37-7.29 (m, 1H), 7.12-7.00 (m, 1H), 6.49 (dt, J=17.1, 2.5 Hz, 1H), 5.80 (dt, J=11.1, 2.4 Hz, 1H), 3.82 (d, J=4.9 Hz, 3H).

### 5-Chloro-1-methyl-1H-imidazo[4,5-b]pyridine-2-carbaldehyde

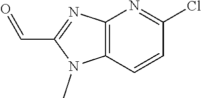

To a stirring solution of 5-chloro-1-methyl-2-vinyl-imidazo[4,5-b]pyridine (180 mg, 0.93 mmol) in a THF/t-butanol/water mixture (18:1:1, 3.6:0.2:0.2 mL) at rt was added N-methyl morpholine N-oxide (141 mg, 1.21 mmol) in one portion, followed by potassium osmate dihydrate (17 mg, 0.05 mmol) in one portion. The reaction was stirred at rt stirred for 16 h. The solvent was removed under reduced pressure, then DCM was added to the vial and there was a precipiate that immediately formed. The suspension was agitated and filtered. The solid was rinsed with minimal DCM and dried to afford the intermediate 1-(5-chloro-1-methyl-imidazo[4,5-b]pyridin-2-yl)ethane-1,2-diol (93 mg, 44% yield, m/z: 228 [M+H]⁺ observed). The crude diol 1-(5-chloro-1-methyl-imidazo[4,5-b]pyridin-2-yl)ethane-1,2-diol (90 mg, 0.57 mmol) was dissolved in a 1:1 dioxane/water mixture (4 mL). To the stirring suspension was added sodium periodate (366 mg, 1.71 mmol) in one portion. After 2h the reaction was quenched by the addition of EtOAc (8 mL) and additional water (5 mL), stirred vigorously and settled. The aqueous phase was extracted with EtOAc (2×5 mL), combined organic layers were dried over anhydrous sodium sulfate, filtered, and concentrated. The crude material was purified by column chromatography 0-50% EtOAc/hexanes to afford 5-chloro-1-methyl-1H-imidazo[4,5-b]pyridine-2-carbaldehyde (50 mg, 45% yield). ¹H NMR (400 MHz, Chloroform-d) δ 10.10 (d, J=0.6 Hz, 1H), 8.17 (d, J=8.5 Hz, 1H), 7.38 (d, J=8.5 Hz, 1H), 4.19 (d, J=0.6 Hz, 3H).

### 5-(2,2′-Dichloro-3′-(5-formyl-6-methoxypyridin-2-yl)-[1,1′-biphenyl]-3-yl)-1-methyl-1H-imidazo[4,5-b]pyridine-2-carbaldehyde

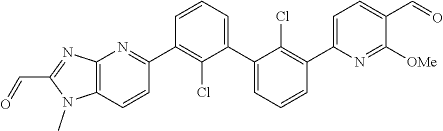 [SMILES: COc1nc(-c2cccc(-c3cccc(-c4ccc5c(n4)nc(C=O)n5C)c3Cl)c2Cl)ccc1C=O]

Pd(PPh₃)₄ (53 mg, 0.05 mmol), potassium carbonate (95 mg, 0.69 mmol), 6-[2-chloro-3-[2-chloro-3-(4,4,5,5-tetramethyl-1,3,2-dioxaborolan-2-yl)phenyl]phenyl]-2-methoxy-pyridine-3-carbaldehyde (133 mg, 0.28 mmol), and 5-chloro-1-methyl-imidazo[4,5-b]pyridine-2-carbaldehyde (45 mg, 0.23 mmol) were suspended in 4:1 mixture of dioxane/water (5 mL) and sparged with N₂ for 5 minutes. The mixture was heated to 95° C. for 45 min, cooled to rt, and evaporated to dryness. The crude oil was purified by silica flash column chromatography 0-60% EtOAc/hexanes to afford 5-(2,2′-dichloro-3′-(5-formyl-6-methoxypyridin-2-yl)-[1,1′-biphenyl]-3-yl)-1-methyl-1H-imidazo[4,5-b]pyridine-2-carbaldehyde (70 mg, 59% yield). ¹H NMR (400 MHz, Chloroform-d) δ 10.42 (d, J=4.6 Hz, 1H), 10.14 (d, J=1.4 Hz, 1H), 8.29 (d, J=8.6 Hz, 1H), 8.19 (d, J=7.7 Hz, 1H), 7.78-7.66 (m, 3H), 7.50-7.38 (m, 5H), 4.29-4.23 (m, 3H), 4.16-4.10 (m, 3H).

### (S)-5-((((6-(2,2′-Dichloro-3′-(1-methyl-2-(((((S)-5-oxopyrrolidin-2-yl)methyl)amino)methyl)-1H-imidazo[4,5-b]pyridin-5-yl)-[1,1′-biphenyl]-3-yl)-2-methoxypyridin-3-yl)methyl)amino)methyl)pyrrolidin-2-one

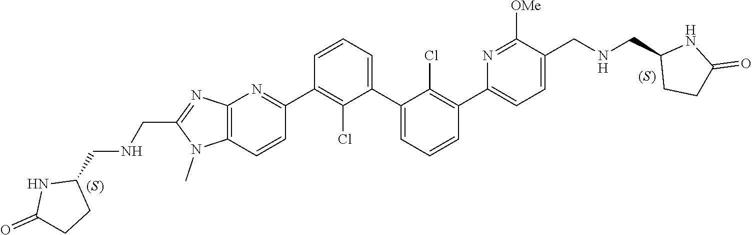

To a vial charged with 5-[2-chloro-3-[2-chloro-3-(5-formyl-6-methoxy-2-pyridyl)phenyl]phenyl]-1-methyl-imidazo[4,5-b]pyridine-2-carbaldehyde (20 mg, 0.04 mmol) and equipped with stirbar was added a 1:1 mixture of THF/MeOH (2 mL), acetic acid 2.21 μL, 0.04 mmol), and (5S)-5-(aminomethyl)pyrrolidin-2-one (18 mg, 0.15 mmol). The suspension went into a uniform solution after 5 minutes of stirring and was left for a total of 2h at rt. To the mixture was added sodium cyanoborohydride (7 mg, 0.12 mmol) in one portion. After 30 minutes the reaction was complete. The solvent was removed under vacuum and the crude material was purified by reverse phase HPLC to afford (S)-5-((((6-(2,2′-dichloro-3′-(1-methyl-2-(((((S)-5-oxopyrrolidin-2-yl)methyl)amino)methyl)-1H-imidazo[4,5-b]pyridin-5-yl)-[1,1′-biphenyl]-3-yl)-2-methoxypyridin-3-yl)methyl)amino)methyl)pyrrolidin-2-one (18.8 mg). m/z: 713 [M]⁺ observed. ¹H NMR (400 MHz, Chloroform-d) δ 8.29 (s, 2H), 8.08 (t, J=7.2 Hz, 1H), 7.88 (t, J=7.0 Hz, 1H), 7.70-7.61 (m, 2H), 7.55 (qd, J=7.2, 3.2 Hz, 3H), 7.47-7.31 (m, 3H), 4.28-4.19 (m, 4H), 4.09 (dd, J=5.7, 2.4 Hz, 3H), 4.07-4.01 (m, 1H), 3.97 (dd, J=5.8, 2.3 Hz, 3H), 3.88-3.81 (m, 1H), 3.19-3.12 (m, 2H), 2.90-2.74 (m, 2H), 2.70-2.63 (m, 1H), 2.43-2.24 (m, 6H), 1.96-1.80 (m, 2H).

### Example 313: (1r,3r)-3-(((6-(2,2′-Dichloro-3′-(2-((((1r,3r)-3-hydroxycyclobutyl)amino)methyl)-1-methyl-1H-imidazo[4,5-b]pyridin-5-yl)-[1,1′-biphenyl]-3-yl)-2-methoxypyridin-3-yl)methyl)amino)cyclobutan-1-ol

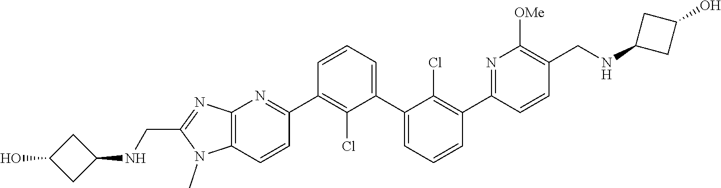 [SMILES: COc1nc(-c2cccc(-c3cccc(-c4ccc5c(n4)nc(CN[C@H]4C[C@H](O)C4)n5C)c3Cl)c2Cl)ccc1CN[C@H]1C[C@H](O)C1]

To a reaction vial equipped with magnetic stirbar was added a premixed solution of 5-[2-chloro-3-[2-chloro-3-(5-formyl-6-methoxy-2-pyridyl)phenyl]phenyl]-1-methyl-imidazo[4,5-b]pyridine-2-carbaldehyde (10 mg, 0.02 mmol) in a 1:1 mixture of MeOH/THF (2 mL) was added acetic acid (1 μL, 0.02 mmol), followed by 3-aminocyclobutanol, (7 mg, 0.08 mmol). The suspension became a uniform solution after 5 minutes of stirring and was stirred for 2h at rt. To the reaction mixture was added sodium cyanoborohydride (4 mg, 0.06 mmol) in one portion. After 30 minutes the reaction was complete The solvent was removed under vacuum and the crude material was purified by reverse phase HPLC to afford (1r,3r)-3-(((6-(2,2′-dichloro-3′-(2-((((1r,3r)-3-hydroxycyclobutyl)amino)methyl)-1-methyl-1H-imidazo[4,5-b]pyridin-5-yl)-[1,1′-biphenyl]-3-yl)-2-methoxypyridin-3-yl)methyl)amino)cyclobutan-1-ol (9.4 mg). m/z: 659 [M]⁺ observed. ¹H NMR (400 MHz, Chloroform-d) δ 8.41 (s, 2H), 8.09 (dd, J=8.2, 2.8 Hz, 1H), 7.86 (dd, J=7.5, 2.7 Hz, 1H), 7.64 (dd, J=6.0, 3.9 Hz, 2H), 7.60-7.48 (m, 3H), 7.42 (dq, J=7.6, 1.7 Hz, 2H), 7.34 (dd, J=7.3, 2.8 Hz, 1H), 4.53-4.43 (m, 2H), 4.23 (d, J=2.8 Hz, 2H), 4.17 (t, J=4.6 Hz, 2H), 4.13-4.06 (m, 3H), 3.95 (dd, J=6.6, 3.0 Hz, 4H), 3.77-3.70 (m, 1H), 2.66 (d, J=2.9 Hz, 2H), 2.53 (dt, J=9.7, 3.0 Hz, 2H), 2.40-2.28 (m, 4H), 2.24 (dd, J=8.2, 4.8 Hz, 2H).

### Example 314: (S)-5-((((6-(2,2′-Dichloro-3′-(3-methyl-2-(((((S)-5-oxopyrrolidin-2-yl)methyl)amino)methyl)-3H-imidazo[4,5-b]pyridin-5-yl)-[1,1′-biphenyl]-3-yl)-2-methoxypyridin-3-yl)methyl)amino)methyl)pyrrolidin-2-one

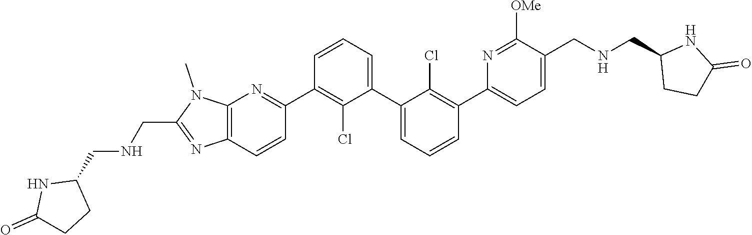 [SMILES: COc1nc(-c2cccc(-c3cccc(-c4ccc5nc(CNC[C@@H]6CCC(=O)N6)n(C)c5n4)c3Cl)c2Cl)ccc1CNC[C@@H]1CCC(=O)N1]

To a vial charged with 5-[2-chloro-3-[2-chloro-3-(5-formyl-6-methoxy-2-pyridyl)phenyl]phenyl]-1-methyl-imidazo[4,5-b]pyridine-2-carbaldehyde (20 mg, 0.04 mmol) and equipped with stir bar was added a 1:1 mixture of THF/MeOH (2 mL), acetic acid 2.21 μL, 0.04 mmol), and (5S)-5-(aminomethyl)pyrrolidin-2-one (18 mg, 0.15 mmol). The suspension went into a uniform solution after 5 minutes of stirring and was left for a total of 2h at rt. To the mixture was added sodium cyanoborohydride (7 mg, 0.12 mmol) in one portion. After 30 minutes the reaction was complete. The solvent was removed under vacuum and the crude material was purified by reverse phase HPLC to afford (S)-5-((((6-(2,2′-dichloro-3′-(3-methyl-2-(((((S)-5-oxopyrrolidin-2-yl)methyl)amino)methyl)-3H-imidazo[4,5-b]pyridin-5-yl)-[1,1′-biphenyl]-3-yl)-2-methoxypyridin-3-yl)methyl)amino)methyl)pyrrolidin-2-one (11.8 mg). m/z: 713 [M]⁺ observed. ¹H NMR (400 MHz, Chloroform-d) δ 8.22 (s, 1H), 8.08 (d, J=8.2 Hz, 1H), 7.89 (d, J=7.5 Hz, 1H), 7.65 (ddd, J=7.8, 3.8, 2.1 Hz, 2H), 7.60-7.49 (m, 3H), 7.47-7.34 (m, 3H), 4.37-4.19 (m, 4H), 4.14-4.02 (m, 4H), 3.97 (d, J=6.9 Hz, 3H), 3.87 (d, J=5.9 Hz, 1H), 3.21 (q, J=6.7 Hz, 2H), 2.98-2.77 (m, 2H), 2.66 (s, 1H), 2.46-2.24 (m, 6H), 1.97-1.82 (m, 2H).

### Example 315: (1r,3r)-3-(((6-(2,2′-Dichloro-3′-(2-((((1r,3r)-3-hydroxycyclobutyl)amino)methyl)-1-methyl-1H-imidazo[4,5-b]pyridin-5-yl)-[1,1′-biphenyl]-3-yl)-2-methoxypyridin-3-yl)methyl)amino)cyclobutan-1-ol

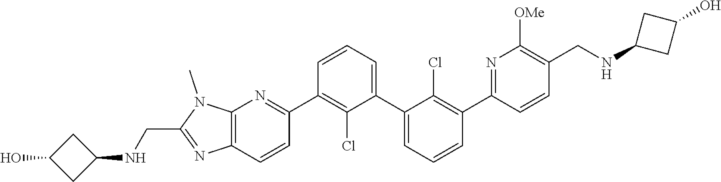 [SMILES: COc1nc(-c2cccc(-c3cccc(-c4ccc5nc(CN[C@H]6C[C@H](O)C6)n(C)c5n4)c3Cl)c2Cl)ccc1CN[C@H]1C[C@H](O)C1]

To a reaction vial equipped with magnetic stir bar was added a premixed solution of 5-[2-chloro-3-[2-chloro-3-(5-formyl-6-methoxy-2-pyridyl)phenyl]phenyl]-1-methyl-imidazo[4,5-b]pyridine-2-carbaldehyde (10 mg, 0.02 mmol) in a 1:1 mixture of MeOH/THF (2 mL) was added acetic acid (1 μL, 0.02 mmol), followed by 3-aminocyclobutanol, (7 mg, 0.08 mmol). The suspension became a uniform solution after 5 minutes of stirring and was stirred for 2h at rt. To the reaction mixture was added sodium cyanoborohydride (4 mg, 0.06 mmol) in one portion. After 30 minutes the reaction was complete The solvent was removed under vacuum and the crude material was purified by reverse phase HPLC to afford (1r,3r)-3-(((6-(2,2′-dichloro-3′-(2-((((1r,3r)-3-hydroxycyclobutyl)amino)methyl)-1-methyl-1H-imidazo[4,5-b]pyridin-5-yl)-[1,1′-biphenyl]-3-yl)-2-methoxypyridin-3-yl)methyl)amino)cyclobutan-1-ol (9.4 mg). m/z: 659 [M]⁺ observed. ¹H NMR (400 MHz, Chloroform-d) δ 8.37 (s, 1H), 8.10 (dd, J=8.2, 1.4 Hz, 1H), 7.87 (dd, J=7.5, 1.4 Hz, 1H), 7.65 (dt, J=7.6, 1.7 Hz, 2H), 7.61-7.48 (m, 3H), 7.43 (dt, J=7.5, 1.7 Hz, 2H), 7.35 (dd, J=7.4, 1.4 Hz, 1H), 4.51-4.44 (m, 2H), 4.30 (d, J=2.8 Hz, 2H), 4.17 (d, J=2.8 Hz, 2H), 4.12-4.06 (m, 3H), 3.94 (d, J=1.4 Hz, 4H), 3.80 (dd, J=9.5, 4.5 Hz, 1H), 2.66 (d, J=1.4 Hz, 1H), 2.58-2.48 (m, 2H), 2.35 (tdd, J=11.0, 6.1, 3.5 Hz, 4H), 2.27 (dd, J=8.6, 5.1 Hz, 2H).

### Example 391: (S)-2-(3-(6-(2,2′-Dichloro-3′-(6-methoxy-5-((((5-oxopyrrolidin-2-yl)methyl)amino)methyl)pyridin-2-yl)-[1,1′-biphenyl]-3-yl)-3-oxo-[1,2,4]triazolo[4,3-a]pyridin-2(3H)-yl)propyl)isoindoline-1,3-dione

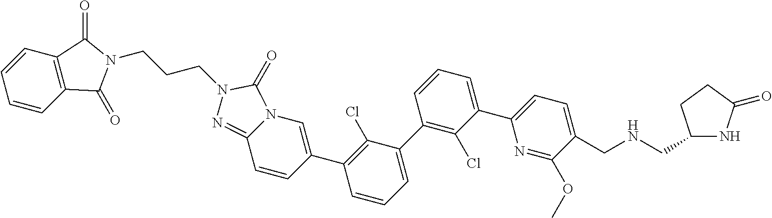 [SMILES: COc1nc(-c2cccc(-c3cccc(-c4ccc5nn(CCCN6C(=O)c7ccccc7C6=O)c(=O)n5c4)c3Cl)c2Cl)ccc1CNC[C@@H]1CCC(=O)N1]

### 2-(3-(6-Bromo-3-oxo-[1,2,4]triazolo[4,3-a]pyridin-2(3H)-yl)propyl)isoindoline-1,3-dione

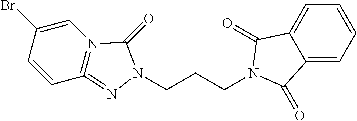

To a stirring brown suspension of 6-bromo-2H-[1,2,4]triazolo[4,3-a]pyridin-3-one (0.94 g, 4.39 mmol) in dry DMF (10 mL) at rt was added cesium carbonate (2.15 g, 6.59 mmol) in one portion, followed by 2-(3-bromopropyl)isoindoline-1,3-dione (1.53 g, 5.71 mmol) dropwise via syringe. The resulting mixture was stirred at rt for 2 days. The reaction suspension was diluted with water (20 mL) and the aqueous phase was extracted with DCM (3×20 mL). The combined organic phases were dried over sodium sulfate, filtered concentrated under reduced pressure. The crude material was purified by normal phase SiO₂ chromatography (0-60% EtOAc/hexane) to afford 2-(3-(6-bromo-3-oxo-[1,2,4]triazolo[4,3-a]pyridin-2(3H)-yl)propyl)isoindoline-1,3-dione (1.4 g, 79% yield) as an off white solid. MS: m/z found 401, 403 [M+H]⁺. ¹H NMR (400 MHz, Chloroform-d) δ 7.93-7.79 (m, 3H), 7.72 (tt, J=5.3, 2.9 Hz, 2H), 7.11-7.03 (m, 1H), 6.93 (t, J=7.2 Hz, 1H), 4.06 (dd, J=7.9, 5.8 Hz, 2H), 3.80 (dd, J=7.8, 5.7 Hz, 2H), 2.33-2.23 (m, 2H).

### tert-Butyl (S)-((6-(2,2′-dichloro-3′-(2-(3-(1,3-dioxoisoindolin-2-yl)propyl)-3-oxo-2,3-dihydro-[1,2,4]triazolo[4,3-a]pyridin-6-yl)-[1,1′-biphenyl]-3-yl)-2-methoxypyridin-3-yl)methyl)((5-oxopyrrolidin-2-yl)methyl)carbamate

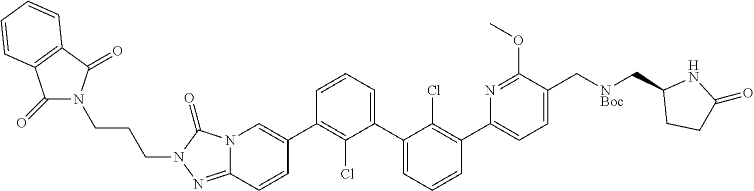 [SMILES: COc1nc(-c2cccc(-c3cccc(-c4ccc5nn(CCCN6C(=O)c7ccccc7C6=O)c(=O)n5c4)c3Cl)c2Cl)ccc1CN(C[C@@H]1CCC(=O)N1)C(=O)OC(C)(C)C]

Pd(PPh₃)₄ (23.61 mg, 0.02 mmol), potassium carbonate (42.31 mg, 0.31 mmol), tert-butyl N-[[6-[2-chloro-3-[2-chloro-3-(4,4,5,5-tetramethyl-1,3,2-dioxaborolan-2-yl)phenyl]phenyl]-2-methoxy-3-pyridyl]methyl]-N-[[(2S)-5-oxopyrrolidin-2-yl]methyl]carbamate (90.66 mg, 0.13 mmol), and 2-[3-(6-bromo-3-oxo-[1,2,4]triazolo[4,3-a]pyridin-2-yl)propyl]isoindoline-1,3-dione (41.00 mg, 0.10 mmol) were suspended in dioxane/water (4:1, 1 mL), then the solution was heated at 95° C. for 30 minutes. Reaction was cooled to rt, diluted with 3 mL water, and extracted with EtOAc (3×2 mL). The combined organics were concentrated under reduced pressure and crude sample was purified by normal phase SiO₂ chromatography (0-10% MeOH/DCM) to afford tert-butyl (S)-((6-(2,2′-dichloro-3′-(2-(3-(1,3-dioxoisoindolin-2-yl)propyl)-3-oxo-2,3-dihydro-[1,2,4]triazolo[4,3-a]pyridin-6-yl)-[1,1′-biphenyl]-3-yl)-2-methoxypyridin-3-yl)methyl)((5-oxopyrrolidin-2-yl)methyl)carbamate (57 mg, 50% yield) as a clear film. MS: m/z found 877 [M+H]⁺.

### (S)-2-(3-(6-(2,2′-Dichloro-3′-(6-methoxy-5-((((5-oxopyrrolidin-2-yl)-methyl)-amino)methyl)-pyridin-2-yl)-[1,1′-biphenyl]-3-yl)-3-oxo-[1,2,4]triazolo[4,3-a]pyridin-2(3H)-yl)propyl)isoindoline-1,3-dione

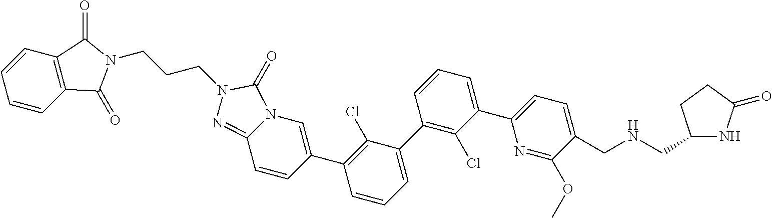 [SMILES: COc1nc(-c2cccc(-c3cccc(-c4ccc5nn(CCCN6C(=O)c7ccccc7C6=O)c(=O)n5c4)c3Cl)c2Cl)ccc1CNC[C@@H]1CCC(=O)N1]

To a stirring solution of tert-butyl N-[[6-[2-chloro-3-[2-chloro-3-[2-[3-(1,3-dioxoisoindolin-2-yl)propyl]-3-oxo-[1,2,4]triazolo[4,3-a]pyridin-6-yl]phenyl]phenyl]-2-methoxy-3-pyridyl]methyl]-N-[[(2S)-5-oxopyrrolidin-2-yl]methyl]carbamate (54 mg, 0.06 mmol) in DCM (2 mL) at rt was added 300 μL of neat TFA dropwise via syringe. The reaction was monitored by LCMS and was complete after 3 h of stirring at rt. The reaction was concentrated under reduced pressure to a crude solid that was purified by reverse phase HPLC to afford (S)-2-(3-(6-(2,2′-dichloro-3′-(6-methoxy-5-((((5-oxopyrrolidin-2-yl)-methyl)-amino)methyl)-pyridin-2-yl)-[1,1′-biphenyl]-3-yl)-3-oxo-[1,2,4]triazolo[4,3-a]pyridin-2(3H)-yl)propyl)isoindoline-1,3-dione (7.6 mg, 16% yield) as a white solid. MS: m/z found 777 [M+H]⁺. ¹H NMR (400 MHz, Chloroform-d) δ 7.84 (d, J=2.4 Hz, 1H), 7.83-7.81 (m, 1H), 7.70 (dt, J=5.1, 2.3 Hz, 2H), 7.67 (d, J=7.7 Hz, 1H), 7.58 (d, J=7.4 Hz, 1H), 7.46-7.35 (m, 4H), 7.35-7.25 (m, 4H), 7.04 (d, J=9.7 Hz, 1H), 6.08 (s, 1H), 4.11 (td, J=7.0, 1.8 Hz, 2H), 4.03 (s, 3H), 3.89-3.82 (m, 2H), 3.80 (s, 2H), 3.78 (s, 1H), 2.79 (ddd, J=12.1, 4.3, 1.8 Hz, 1H), 2.56 (dd, J=11.8, 8.9 Hz, 1H), 2.33 (ddd, J=24.0, 8.2, 6.5 Hz, 4H), 2.24 (ddd, J=13.0, 7.4, 1.7 Hz, 1H), 1.25 (s, 2H), 0.82 (s, 1H).

### Example 392: (S)-6-(2,2′-Dichloro-3′-(6-methoxy-5-((((5-oxopyrrolidin-2-yl)methyl)-amino)-methyl)pyridin-2-yl)-[1,1′-biphenyl]-3-yl)-2-(3-(diisobutylamino)propyl)-[1,2,4]triazolo[4,3-a]pyridin-3(2H)-one

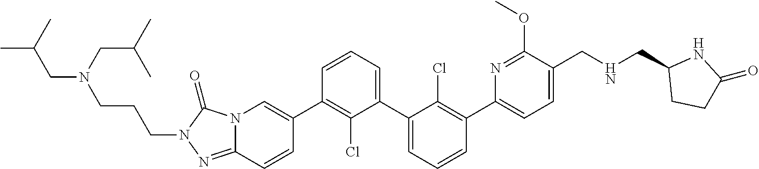 [SMILES: COc1nc(-c2cccc(-c3cccc(-c4ccc5nn(CCCN(CC(C)C)CC(C)C)c(=O)n5c4)c3Cl)c2Cl)ccc1CNC[C@@H]1CCC(=O)N1]

### 6-Bromo-2-(3-(diisobutylamino)propyl)-[1,2,4]triazolo[4,3-a]pyridin-3(2H)-one

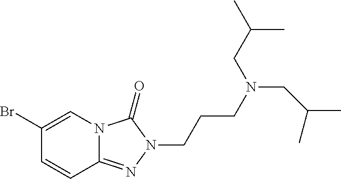 [SMILES: CC(C)CN(CCCn1nc2ccc(Br)cn2c1=O)CC(C)C]

To a stirring solution of 2-(3-aminopropyl)-6-bromo-[1,2,4]triazolo[4,3-a]pyridin-3-one (59 mg, 0.22 mmol) in a 1:1 mixture of THF/MeOH (5 mL) was added acetic acid (0.01 mL, 13 mg, 0.22 mmol) dropwise, followed by 2-methylpropanal (0.06 mL, 47 mg, 0.65 mmol) via syringe. The reaction mixture was stirred at rt for 1 h and sodium cyanoborohydride (27 mg, 0.44 mmol) was added in one portion, then the mixture was further stirred at rt for 1 hour. Reaction was subsequently diluted with water (5 mL) and extracted with DCM (3×5 mL). The combined organics were further washed with brine (10 mL), dried over sodium sulfate, filtered, and concentrated under reduced pressure to afford 6-bromo-2-(3-(diisobutylamino)propyl)-[1,2,4]triazolo[4,3-a]pyridin-3(2H)-one as a yellow oil (52 mg, 62.3% yield). MS: m/z found 384 [M+H]. Material was used for the next step without further purification.

### tert-Butyl (S)-((6-(2,2′-dichloro-3′-(2-(3-(diisobutylamino)propyl)-3-oxo-2,3-dihydro-[1,2,4]triazolo[4,3-a]pyridin-6-yl)-[1,1′-biphenyl]-3-yl)-2-methoxypyridin-3-yl)methyl)((5-oxopyrrolidin-2-yl)methyl)carbamate

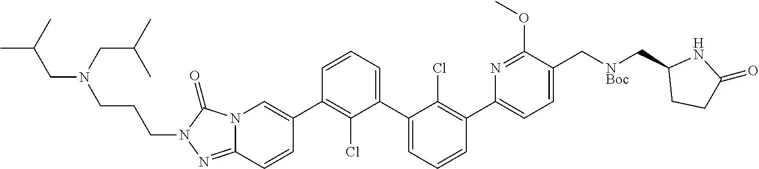 [SMILES: COc1nc(-c2cccc(-c3cccc(-c4ccc5nn(CCCN(CC(C)C)CC(C)C)c(=O)n5c4)c3Cl)c2Cl)ccc1CN(C[C@@H]1CCC(=O)N1)C(=O)OC(C)(C)C]

Pd(PPh₃)₄ (31 mg, 0.03 mmol), potassium carbonate (56 mg, 0.41 mmol), tert-butyl N-[[6-[2-chloro-3-[2-chloro-3-(4,4,5,5-tetramethyl-1,3,2-dioxaborolan-2-yl)phenyl]phenyl]-2-methoxy-3-pyridyl]methyl]-N-[[(2S)-5-oxopyrrolidin-2-yl]methyl]carbamate (120 mg, 0.18 mmol), and 6-bromo-2-[3-(diisobutylamino)propyl]-[1,2,4]triazolo[4,3-a]pyridin-3-one (52 mg, 0.14 mmol) were suspended in dioxane/water (4:1, 4 mL), then the solution was heated at 95° C. for 30 minutes. Reaction was cooled to rt, diluted with 6 mL water, and extracted with EtOAc (3×3 mL). The combined organics were concentrated under reduced pressure and crude sample was purified by normal phase SiO₂ chromatography (0-4% MeOH/DCM) to afford tert-butyl (S)-((6-(2,2′-dichloro-3′-(2-(3-(diisobutylamino)propyl)-3-oxo-2,3-dihydro-[1,2,4]triazolo[4,3-a]pyridin-6-yl)-[1,1′-biphenyl]-3-yl)-2-methoxypyridin-3-yl)methyl)((5-oxopyrrolidin-2-yl)methyl)carbamate as a yellow oil. (91 mg, 78% yield). MS: m/z found 859 [M+H].

### (S)-6-(2,2′-Dichloro-3′-(6-methoxy-5-((((5-oxopyrrolidin-2-yl)methyl)-amino)-methyl)pyridin-2-yl)-[1,1′-biphenyl]-3-yl)-2-(3-(diisobutylamino)propyl)-[1,2,4]triazolo[4,3-a]pyridin-3(2H)-one

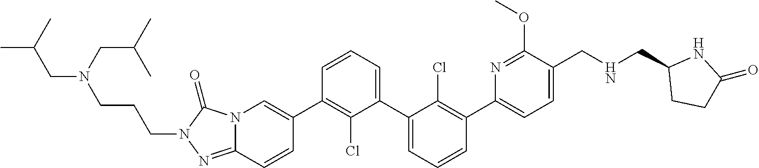 [SMILES: COc1nc(-c2cccc(-c3cccc(-c4ccc5nn(CCCN(CC(C)C)CC(C)C)c(=O)n5c4)c3Cl)c2Cl)ccc1CNC[C@@H]1CCC(=O)N1]

To a stirring solution of tert-butyl N-[[6-[2-chloro-3-[2-chloro-3-[2-[3-(diisobutylamino)propyl]-3-oxo-[1,2,4]triazolo[4,3-a]pyridin-6-yl]phenyl]phenyl]-2-methoxy-3-pyridyl]methyl]-N-[[(2S)-5-oxopyrrolidin-2-yl]methyl]carbamate (140 mg, 0.11 mmol) in DCM at rt was added 500 μL of neat TFA dropwise via syringe. The reaction was monitored by LCMS and was complete after 3 h of stirring at rt. The reaction was concentrated under reduced pressure to a crude solid that was purified by reverse phase HPLC to afford (S)-6-(2,2′-dichloro-3′-(6-methoxy-5-((((5-oxopyrrolidin-2-yl)methyl)-amino)-methyl)pyridin-2-yl)-[1,1′-biphenyl]-3-yl)-2-(3-(diisobutylamino)propyl)-[1,2,4]triazolo[4,3-a]pyridin-3(2H)-one. m/z: 759 [M]⁺ observed. ¹H NMR (400 MHz, Chloroform-d) δ 7.90-7.84 (m, 1H), 7.67 (ddd, J=7.7, 3.4, 1.8 Hz, 1H), 7.59 (dd, J=7.4, 3.1 Hz, 1H), 7.41 (ddd, J=15.2, 7.8, 3.0 Hz, 4H), 7.34-7.26 (m, 3H), 7.15 (ddd, J=9.6, 3.4, 1.2 Hz, 1H), 6.26 (s, 1H), 4.06 (s, 2H), 4.03-4.01 (m, 3H), 3.82 (d, J=2.9 Hz, 2H), 3.78 (s, 1H), 2.80 (dt, J=11.9, 3.6 Hz, 1H), 2.58 (ddt, J=11.8, 8.5, 4.1 Hz, 1H), 2.45 (q, J=4.3 Hz, 2H), 2.36 (td, J=7.5, 6.9, 3.3 Hz, 2H), 2.30-2.20 (m, 1H), 2.08 (dd, J=7.2, 2.9 Hz, 4H), 2.03-1.94 (m, 3H), 1.79-1.65 (m, 3H), 0.88 (dd, J=6.7, 3.1 Hz, 12H).

### Example 338: (5S)-5-[[[6-[2-Chloro-3-[2-chloro-3-[2-[[[(2S)-5-oxopyrrolidin-2-yl]methylamino]methyl]imidazo[1,2-b]pyridazin-6-yl]phenyl]phenyl]-2-methoxy-3-pyridyl]methylamino]methyl]pyrrolidin-2-one

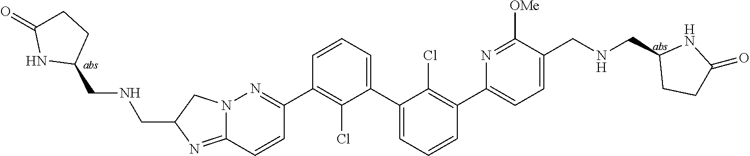 [SMILES: COc1nc(-c2cccc(-c3cccc(C4=NN5CC(CNC[C@@H]6CCC(=O)N6)N=C5C=C4)c3Cl)c2Cl)ccc1CNC[C@@H]1CCC(=O)N1]

### 6-[2-Chloro-3-[2-chloro-3-(5-formyl-6-methoxy-2-pyridyl)phenyl]phenyl]imidazo[1,2-b]pyridazine-2-carbaldehyde

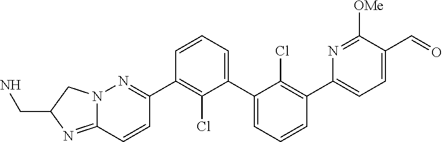 [SMILES: COc1nc(-c2cccc(-c3cccc(C4=NN5CC(CN)N=C5C=C4)c3Cl)c2Cl)ccc1C=O]

To a solution of 6-chloroimidazo[1,2-b]pyridazine-2-carbaldehyde (50 mg, 0.28 mmol), 6-[2-chloro-3-[2-chloro-3-(4,4,5,5-tetramethyl-1,3,2-dioxaborolan-2-yl)phenyl]phenyl]-2-methoxy-pyridine-3-carbaldehyde (133 mg, 0.28 mmol) and potassium carbonate (114 mg, 0.83 mmol) in degassed dioxane/water (6:1) was added Pd(PPh₃)₄ (31.82 mg, 0.03 mmol) and the mixture was stirred at 90° C. for 2 hours under N₂(g). Water was added and the mixture was extracted into EtOAc three times. The combined organic layers were dried over anhydrous sodium sulfate, concentrated, and purified on silica gel column using EtOAc in hexanes (0 to 20% gradient) to provide 6-[2-chloro-3-[2-chloro-3-(5-formyl-6-methoxy-2-pyridyl)phenyl]phenyl]imidazo[1,2-b]pyridazine-2-carbaldehyde (95 mg, 68.5%). MS: m/z found 503.1 [M+H]⁺.

### (5S)-5-[[[6-[2-Chloro-3-[2-chloro-3-[2-[[[(2S)-5-oxopyrrolidin-2-yl]methylamino]methyl]imidazo[1,2-b]pyridazin-6-yl]phenyl]phenyl]-2-methoxy-3-pyridyl]methylamino]methyl]pyrrolidin-2-one

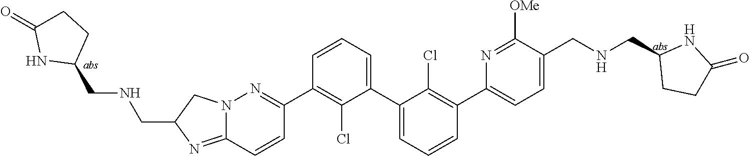 [SMILES: COc1nc(-c2cccc(-c3cccc(C4=NN5CC(CNC[C@@H]6CCC(=O)N6)N=C5C=C4)c3Cl)c2Cl)ccc1CNC[C@@H]1CCC(=O)N1]

A mixture of 6-[2-chloro-3-[2-chloro-3-(5-formyl-6-methoxy-2-pyridyl)phenyl]phenyl]imidazo[1,2-b]pyridazine-2-carbaldehyde (25 mg, 0.05 mmol), (5S)-5-(aminomethyl)pyrrolidin-2-one (23 mg, 0.20 mmol) and acetic acid (6 uL, 6 mg, 0.10 mmol) in MeOH/THF (1:1) was stirred for 1.5 hour at rt. NaBH₃CN (12.5 mg, 0.20 mmol) was added, the mixture was stirred for additional 1 h, quenched with water and directly purified by reversed phase chromatography to give (5S)-5-[[[6-[2-chloro-3-[2-chloro-3-[2-[[[(2S)-5-oxopyrrolidin-2-yl]methylamino]methyl]imidazo[1,2-b]pyridazin-6-yl]phenyl]phenyl]-2-methoxy-3-pyridyl]methylamino]methyl]pyrrolidin-2-one (27 mg, 77.7%). MS: m/z found 699.2 [M+H]⁺. ¹H NMR (400 MHz, Methanol-d₄): δ 8.20 (d, J=0.7 Hz, 1H), 8.05 (dt, J=9.4, 0.6 Hz, 1H), 7.79 (d, J=7.5 Hz, 1H), 7.70-7.63 (m, 2H), 7.58 (dd, J=7.9, 7.4 Hz, 1H), 7.54-7.49 (m, 3H), 7.41 (dd, J=7.6, 1.7 Hz, 1H), 7.29 (d, J=7.5 Hz, 1H), 4.17-4.07 (m, 2H), 4.04 (s, 3H), 3.99 (d, J=3.6 Hz, 2H), 3.94-3.84 (m, 2H), 2.86 (qdd, J=12.3, 6.8, 5.3 Hz, 4H), 2.41-2.27 (m, 6H), 1.91-1.79 (m, 2H).

### Example 339: 2-[[6-[2-Chloro-3-[2-chloro-3-[2-[[2-hydroxyethyl(methyl)amino]methyl]imidazo[1,2-b]pyridazin-6-yl]phenyl]phenyl]-2-methoxy-3-pyridyl]methyl-methyl-amino]etanol

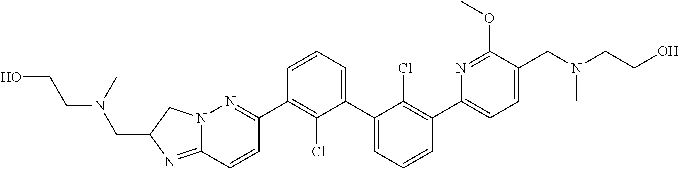 [SMILES: COc1nc(-c2cccc(-c3cccc(C4=NN5CC(CN(C)CCO)N=C5C=C4)c3Cl)c2Cl)ccc1CN(C)CCO]

A mixture of 6-[2-chloro-3-[2-chloro-3-(5-formyl-6-methoxy-2-pyridyl)phenyl]phenyl]imidazo[1,2-b]pyridazine-2-carbaldehyde (25 mg, 0.05 mmol), 2-(methylamino)ethanol (15 mg, 0.20 mmol) and acetic acid (6 uL, 6 mg, 0.10 mmol) in MeOH/THF (1:1) was stirred for 1.5 hour at rt. NaBH₃CN (12.5 mg, 0.20 mmol) was added, the mixture was stirred for additional 1 h, quenched with water and directly purified by reversed phase chromatography to give 2-[[6-[2-chloro-3-[2-chloro-3-[2-[[2-hydroxyethyl(methyl)amino]methyl]imidazo[1,2-b]pyridazin-6-yl]phenyl]phenyl]-2-methoxy-3-pyridyl]methyl-methyl-amino]ethanol (23 mg, 74.5%). MS: m/z found 621.2 [M+H]⁺. ¹H NMR (400 MHz, Methanol-d₄): δ 8.25 (s, 1H), 8.05 (dd, J=9.5, 0.7 Hz, 1H), 7.85 (d, J=7.5 Hz, 1H), 7.68 (ddd, J=7.6, 1.8, 0.8 Hz, 2H), 7.59 (t, J=7.6 Hz, 1H), 7.54-7.50 (m, 3H), 7.42 (dd, J=7.6, 1.7 Hz, 1H), 7.31 (d, J=7.5 Hz, 1H), 4.03 (s, 3H), 4.03 (s, 2H), 3.97 (s, 2H), 3.81 (t, J=5.7 Hz, 2H), 3.77 (t, J=5.7 Hz, 2H), 2.92 (t, J=5.7 Hz, 2H), 2.83 (t, J=5.7 Hz, 2H), 2.55 (s, 3H), 2.51 (s, 3H).

### Example 340: (2S)-1-[[6-[2-Chloro-3-[2-chloro-3-[2-[[[(2S)-2-hydroxypropyl]amino]methyl]imidazo[1,2-b]pyridazin-6-yl]phenyl]phenyl]-2-methoxy-3-pyridyl]methylamino]propan-2-ol

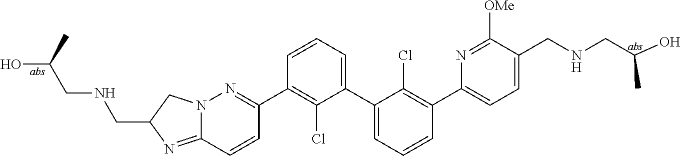 [SMILES: COc1nc(-c2cccc(-c3cccc(C4=NN5CC(CNC[C@H](C)O)N=C5C=C4)c3Cl)c2Cl)ccc1CNC[C@H](C)O]

A mixture of 6-[2-chloro-3-[2-chloro-3-(5-formyl-6-methoxy-2-pyridyl)phenyl]phenyl]imidazo[1,2-b]pyridazine-2-carbaldehyde (25 mg, 0.05 mmol), (2S)-1-aminopropan-2-ol (15 mg, 0.20 mmol) and acetic acid (6 uL, 6 mg, 0.10 mmol) in MeOH/THF (1:1) was stirred for 1.5 hour at rt. NaBH₃CN (12.5 mg, 0.20 mmol) was added, the mixture was stirred for additional 1 h, quenched with water and directly purified by reversed phase chromatography to give (2S)-1-[[6-[2-chloro-3-[2-chloro-3-[2-[[[(2S)-2-hydroxypropyl]amino]methyl]imidazo[1,2-b]pyridazin-6-yl]phenyl]phenyl]-2-methoxy-3-pyridyl]methylamino]propan-2-ol (25 mg, 81.0%). MS: m/z found 621.2 [M+H]⁺. ¹H NMR (400 MHz, Methanol-d₄): δ 8.24 (d, J=0.7 Hz, 1H), 8.06 (dd, J=9.5, 0.7 Hz, 1H), 7.84-7.78 (m, 1H), 7.68 (t, J=1.9 Hz, 1H), 7.66 (dd, J=2.2, 1.8 Hz, 1H), 7.59 (t, J=7.6 Hz, 1H), 7.55-7.50 (m, 3H), 7.42 (dd, J=7.6, 1.8 Hz, 1H), 7.31 (d, J=7.4 Hz, 1H), 4.23-4.19 (m, 2H), 4.14-4.07 (m, 2H), 4.06 (s, 3H), 4.03-3.95 (m, 2H), 2.91 (ddd, J=12.3, 3.3, 0.8 Hz, 2H), 2.76 (ddd, J=12.4, 9.2, 5.2 Hz, 2H), 1.21 (d, J=4.7 Hz, 3H), 1.20 (d, J=4.7 Hz, 3H).

### Example 352: (5S)-5-[[[6-[2-Chloro-3-[2-chloro-3-[2-[(2-hydroxyethylamino)methyl]imidazo[1,2-b]pyridazin-6-yl]phenyl]phenyl]-2-methoxy-3-pyridyl]methylamino]methyl]pyrrolidin-2-one

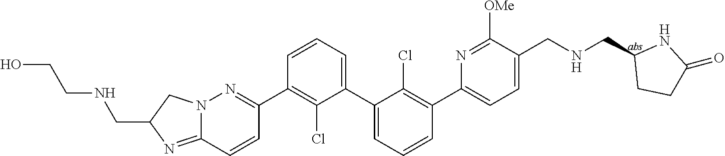 [SMILES: COc1nc(-c2cccc(-c3cccc(C4=NN5CC(CNCCO)N=C5C=C4)c3Cl)c2Cl)ccc1CNC[C@@H]1CCC(=O)N1]

### tert-Butyl N-[[6-[2-chloro-3-[2-chloro-3-(2-formylimidazo[1,2-b]pyridazin-6-yl)phenyl]phenyl]-2-methoxy-3-pyridyl]methyl]-N-[[(2S)-5-oxopyrrolidin-2-yl]methyl]carbamate

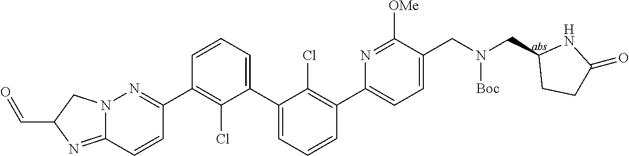 [SMILES: COc1nc(-c2cccc(-c3cccc(C4=NN5CC(C=O)N=C5C=C4)c3Cl)c2Cl)ccc1CN(C[C@@H]1CCC(=O)N1)C(=O)OC(C)(C)C]

To a solution of 6-chloroimidazo[1,2-b]pyridazine-2-carbaldehyde (30 mg, 0.17 mmol), tert-butyl N-[[6-[2-chloro-3-[2-chloro-3-(4,4,5,5-tetramethyl-1,3,2-dioxaborolan-2-yl)phenyl]phenyl]-2-methoxy-3-pyridyl]methyl]-N-[[(2S)-5-oxopyrrolidin-2-yl]methyl]carbamate (113 mg, 0.17 mmol) and potassium carbonate (69 mg, 0.50 mmol) in degassed dioxane/water (6:1) was added Pd(PPh₃)₄ (19 mg, 0.02 mmol) and the mixture was stirred at 90° C. for 2 hours under N₂(g). Water was added and the mixture was extracted into EtOAc three times. The combined organic layers were dried over anhydrous sodium sulfate, concentrated, and purified on silica gel column using EtOAc in hexanes (0 to 20% gradient) to provide tert-butyl N-[[6-[2-chloro-3-[2-chloro-3-(2-formylimidazo[1,2-b]pyridazin-6-yl)phenyl]phenyl]-2-methoxy-3-pyridyl]methyl]-N-[[(2S)-5-oxopyrrolidin-2-yl]methyl]carbamate (101 mg, 87.1%). MS: m/z found 701.2 [M+H]⁺.

### tert-Butyl N-[[6-[2-chloro-3-[2-chloro-3-[2-[(2-hydroxyethylamino)methyl]imidazo[1,2-b]pyridazin-6-yl]phenyl]phenyl]-2-methoxy-3-pyridyl]methyl]-N-[[(2S)-5-oxopyrrolidin-2-yl]methyl]carbamate

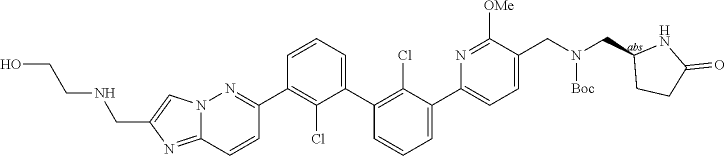 [SMILES: COc1nc(-c2cccc(-c3cccc(-c4ccc5nc(CNCCO)cn5n4)c3Cl)c2Cl)ccc1CN(C[C@@H]1CCC(=O)N1)C(=O)OC(C)(C)C]

A mixture of tert-butyl N-[[6-[2-chloro-3-[2-chloro-3-(2-formylimidazo[1,2-b]pyridazin-6-yl)phenyl]phenyl]-2-methoxy-3-pyridyl]methyl]-N-[[(2S)-5-oxopyrrolidin-2-yl]methyl]carbamate (25 mg, 0.04 mmol), 2-aminoethanol (4 mg, 0.07 mmol) and acetic acid (2 uL, 2.1 mg, 0.04 mmol) in MeOH/THF (2:1) was stirred for 1 hour at rt. NaBH₃CN (4.5 mg, 0.07 mmol) was added, the mixture was stirred for 1 hour, quenched with water, and purified by reversed phase chromatography to give tert-butyl N-[[6-[2-chloro-3-[2-chloro-3-[2-[(2-hydroxyethylamino)methyl]imidazo[1,2-b]pyridazin-6-yl]phenyl]phenyl]-2-methoxy-3-pyridyl]methyl]-N-[[(2S)-5-oxopyrrolidin-2-yl]methyl]carbamate (15 mg, 56.4%). MS: m/z found 746.3 [M+H]⁺.

### (5S)-5-[[[6-[2-Chloro-3-[2-chloro-3-[2-[(2-hydroxyethylamino)methyl]imidazo[1,2-b]pyridazin-6-yl]phenyl]phenyl]-2-methoxy-3-pyridyl]methylamino]methyl]pyrrolidin-2-one

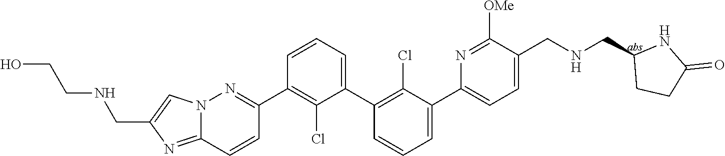 [SMILES: COc1nc(-c2cccc(-c3cccc(-c4ccc5nc(CNCCO)cn5n4)c3Cl)c2Cl)ccc1CNC[C@@H]1CCC(=O)N1]

A solution of tert-butyl N-[[6-[2-chloro-3-[2-chloro-3-[2-[(2-hydroxyethylamino)methyl]imidazo[1,2-b]pyridazin-6-yl]phenyl]phenyl]-2-methoxy-3-pyridyl]methyl]-N-[[(2S)-5-oxopyrrolidin-2-yl]methyl]carbamate (20 mg, 0.03 mmol) in DCM/TFA (2:1) was stirred for 30 minutes, concentrated and purified by reversed phase chromatography to give (5S)-5-[[[6-[2-chloro-3-[2-chloro-3-[2-[(2-hydroxyethylamino)methyl]imidazo[1,2-b]pyridazin-6-yl]phenyl]phenyl]-2-methoxy-3-pyridyl]methylamino]methyl]pyrrolidin-2-one (17 mg, 98.2%). MS: m/z found 646.2 [M+H]⁺. ¹H NMR (400 MHz, Methanol-d₄): δ 8.22 (s, 1H), 8.05 (dd, J=9.5, 0.7 Hz, 1H), 7.74 (d, J=7.4 Hz, 1H), 7.67 (ddd, J=7.7, 5.2, 1.8 Hz, 2H), 7.58 (t, J=7.6 Hz, 1H), 7.54-7.48 (m, 3H), 7.40 (dd, J=7.6, 1.8 Hz, 1H), 7.25 (d, J=7.4 Hz, 1H), 4.17 (s, 2H), 4.02 (s, 3H), 3.89-3.79 (m, 3H), 3.77-3.72 (m, 2H), 2.95 (t, J=5.4 Hz, 2H), 2.76-2.64 (m, 2H), 2.37-2.23 (m, 3H), 1.87-1.77 (m, 1H).

### Example 353: (5S)-5-[[[6-[2-Chloro-3-[2-chloro-3-[2-[[2-hydroxyethyl(methyl)amino]methyl]imidazo[1,2-b]pyridazin-6-yl]phenyl]phenyl]-2-methoxy-3-pyridyl]methylamino]methyl]pyrrolidin-2-one

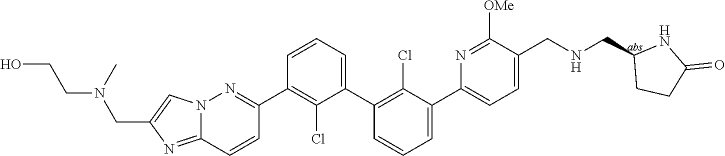 [SMILES: COc1nc(-c2cccc(-c3cccc(-c4ccc5nc(CN(C)CCO)cn5n4)c3Cl)c2Cl)ccc1CNC[C@@H]1CCC(=O)N1]

### tert-Butyl N-[[6-[2-chloro-3-[2-chloro-3-[2-[[2-hydroxyethyl(methyl)amino]methyl]imidazo[1,2-b]pyridazin-6-yl]phenyl]phenyl]-2-methoxy-3-pyridyl]methyl]-N-[[(2S)-5-oxopyrrolidin-2-yl]methyl]carbamate

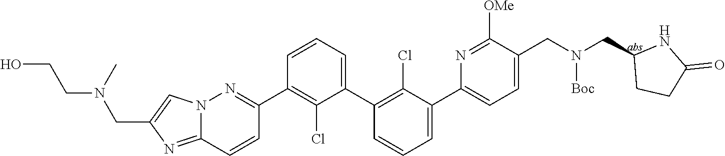 [SMILES: COc1nc(-c2cccc(-c3cccc(-c4ccc5nc(CN(C)CCO)cn5n4)c3Cl)c2Cl)ccc1CN(C[C@@H]1CCC(=O)N1)C(=O)OC(C)(C)C]

A mixture of tert-butyl N-[[6-[2-chloro-3-[2-chloro-3-(2-formylimidazo[1,2-b]pyridazin-6-yl)phenyl]phenyl]-2-methoxy-3-pyridyl]methyl]-N-[[(2S)-5-oxopyrrolidin-2-yl]methyl]carbamate (25 mg, 0.04 mmol), 2-(methylamino)ethanol (5.4 mg, 0.07 mmol) and acetic acid (2. uL, 2.1 mg, 0.04 mmol) in MeOH/THF (2:1) was stirred for 1 hour at rt. NaBH₃CN (4.5 mg, 0.07 mmol) was added, the mixture was stirred for 1 hour, quenched with water, and purified by reversed phase chromatography to give tert-butyl N-[[6-[2-chloro-3-[2-chloro-3-[2-[[2-hydroxyethyl(methyl)amino]methyl]imidazo[1,2-b]pyridazin-6-yl]phenyl]phenyl]-2-methoxy-3-pyridyl]methyl]-N-[[(2S)-5-oxopyrrolidin-2-yl]methyl]carbamate (23 mg, 84.9%). MS: m/z found 760.3 [M+H]⁺.

### (5S)-5-[[[6-[2-Chloro-3-[2-chloro-3-[2-[[2-hydroxyethyl(methyl)amino]methyl]imidazo[1,2-b]pyridazin-6-yl]phenyl]phenyl]-2-methoxy-3-pyridyl]methylamino]methyl]pyrrolidin-2-one

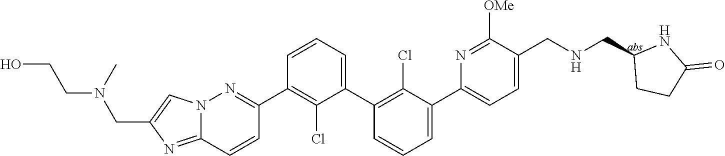 [SMILES: COc1nc(-c2cccc(-c3cccc(-c4ccc5nc(CN(C)CCO)cn5n4)c3Cl)c2Cl)ccc1CNC[C@@H]1CCC(=O)N1]

A solution of tert-butyl N-[[6-[2-chloro-3-[2-chloro-3-[2-[[2-hydroxyethyl(methyl)amino]methyl]imidazo[1,2-b]pyridazin-6-yl]phenyl]phenyl]-2-methoxy-3-pyridyl]methyl]-N-[[(2S)-5-oxopyrrolidin-2-yl]methyl]carbamate (20 mg, 0.03 mmol) in DCM/TFA (2:1) was stirred for 30 minutes, concentrated and purified by reversed phase chromatography to give (5S)-5-[[[6-[2-chloro-3-[2-chloro-3-[2-[[2-hydroxyethyl(methyl)amino]methyl]imidazo[1,2-b]pyridazin-6-yl]phenyl]phenyl]-2-methoxy-3-pyridyl]methylamino]methyl]pyrrolidin-2-one (17 mg, 97.9%). MS: m/z found 660.2 [M+H]⁺. ¹H NMR (400 MHz, Methanol-d₄): δ 8.23 (s, 1H), 8.04 (dd, J=9.5, 0.7 Hz, 1H), 7.74 (d, J=7.5 Hz, 1H), 7.67 (ddd, J=7.9, 6.2, 1.8 Hz, 2H), 7.58 (t, J=7.6 Hz, 1H), 7.54-7.48 (m, 3H), 7.40 (dd, J=7.6, 1.7 Hz, 1H), 7.25 (d, J=7.4 Hz, 1H), 4.02 (s, 3H), 3.95 (s, 2H), 3.89-3.81 (m, 3H), 3.75 (t, J=5.8 Hz, 2H), 2.76 (t, J=5.7 Hz, 2H), 2.74-2.65 (m, 2H), 2.45 (s, 3H), 2.37-2.24 (m, 3H), 1.87-1.77 (m, 1H).

### Example 354: (5S)-5-[[[6-[2-Chloro-3-[2-chloro-3-[2-[[[(2S)-2-hydroxypropyl]amino]methyl]imidazo[1,2-b]pyridazin-6-yl]phenyl]phenyl]-2-methoxy-3-pyridyl]methylamino]methyl]pyrrolidin-2-one

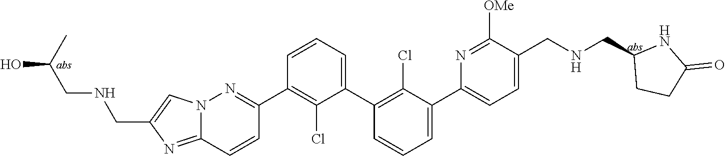 [SMILES: COc1nc(-c2cccc(-c3cccc(-c4ccc5nc(CNC[C@H](C)O)cn5n4)c3Cl)c2Cl)ccc1CNC[C@@H]1CCC(=O)N1]

### tert-Butyl N-[[6-[2-chloro-3-[2-chloro-3-[2-[[[(2S)-2-hydroxypropyl]amino]methyl]imidazo[1,2-b]pyridazin-6-yl]phenyl]phenyl]-2-methoxy-3-pyridyl]methyl]-N-[[(2S)-5-oxopyrrolidin-2-yl]methyl]carbamate

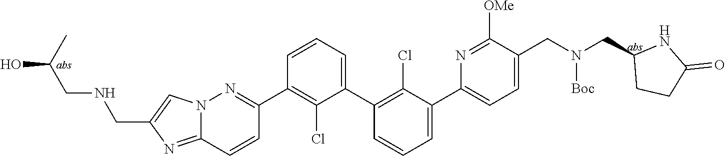 [SMILES: COc1nc(-c2cccc(-c3cccc(-c4ccc5nc(CNC[C@H](C)O)cn5n4)c3Cl)c2Cl)ccc1CN(C[C@@H]1CCC(=O)N1)C(=O)OC(C)(C)C]

A mixture of tert-butyl N-[[6-[2-chloro-3-[2-chloro-3-(2-formylimidazo[1,2-b]pyridazin-6-yl)phenyl]phenyl]-2-methoxy-3-pyridyl]methyl]-N-[[(2S)-5-oxopyrrolidin-2-yl]methyl]carbamate (25 mg, 0.04 mmol), (2S)-1-aminopropan-2-ol (5.4 mg, 0.07 mmol) and acetic acid (2. uL, 2.1 mg, 0.04 mmol) in MeOH/THF (2:1) was stirred for 1 hour at rt. NaBH₃CN (4.5 mg, 0.07 mmol) was added, the mixture was stirred for 1 hour, quenched with water, and purified by reversed phase chromatography to give tert-butyl N-[[6-[2-chloro-3-[2-chloro-3-[2-[[[(2S)-2-hydroxypropyl]amino]methyl]imidazo[1,2-b]pyridazin-6-yl]phenyl]phenyl]-2-methoxy-3-pyridyl]methyl]-N-[[(2S)-5-oxopyrrolidin-2-yl]methyl]carbamate (22 mg, 81.2%). MS: m/z found 760.3 [M+H]⁺.

### (5S)-5-[[[6-[2-Chloro-3-[2-chloro-3-[2-[[[(2S)-2-hydroxypropyl]amino]methyl]imidazo[1,2-b]pyridazin-6-yl]phenyl]phenyl]-2-methoxy-3-pyridyl]methylamino]methyl]pyrrolidin-2-one

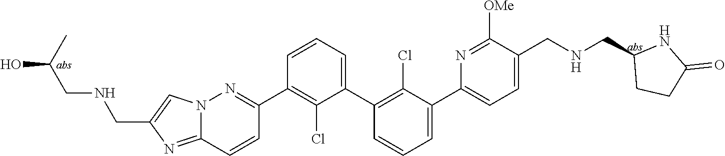 [SMILES: COc1nc(-c2cccc(-c3cccc(-c4ccc5nc(CNC[C@H](C)O)cn5n4)c3Cl)c2Cl)ccc1CNC[C@@H]1CCC(=O)N1]

A solution of tert-butyl N-[[6-[2-chloro-3-[2-chloro-3-[2-[[[(2S)-2-hydroxypropyl]amino]methyl]imidazo[1,2-b]pyridazin-6-yl]phenyl]phenyl]-2-methoxy-3-pyridyl]methyl]-N-[[(2S)-5-oxopyrrolidin-2-yl]methyl]carbamate (20 mg, 0.03 mmol) in DCM/TFA (2:1) was stirred for 30 minutes, concentrated and purified by reversed phase chromatography to give (5S)-5-[[[6-[2-chloro-3-[2-chloro-3-[2-[[[(2S)-2-hydroxypropyl]amino]methyl]imidazo[1,2-b]pyridazin-6-yl]phenyl]phenyl]-2-methoxy-3-pyridyl]methylamino]methyl]pyrrolidin-2-one (17 mg, 97.9%). MS: m/z found 660.2 [M+H]⁺. ¹H NMR (400 MHz, Methanol-d₄): δ 8.22 (s, 1H), 8.11-7.99 (m, 1H), 7.74 (d, J=7.5 Hz, 1H), 7.67 (ddd, J=7.8, 5.1, 1.8 Hz, 2H), 7.58 (t, J=7.6 Hz, 1H), 7.54-7.48 (m, 3H), 7.40 (dd, J=7.6, 1.8 Hz, 1H), 7.25 (d, J=7.4 Hz, 1H), 4.24-4.10 (m, 2H), 4.02 (s, 3H), 3.96 (ddd, J=9.4, 6.2, 3.4 Hz, 1H), 3.88-3.78 (m, 3H), 2.85 (dd, J=12.2, 3.5 Hz, 1H), 2.76-2.63 (m, 3H), 2.39-2.23 (m, 3H), 1.87-1.75 (m, 1H), 1.19 (d, J=6.3 Hz, 3H).

### Example 355: (3S)-4-[[6-[2-Chloro-3-[2-chloro-3-[6-methoxy-5-[[[(2S)-5-oxopyrrolidin-2-yl]methylamino]methyl]-2-pyridyl]phenyl]phenyl]imidazo[1,2-b]pyridazin-2-yl]methylamino]-3-hydroxy-N,N-dimethyl-butanamide

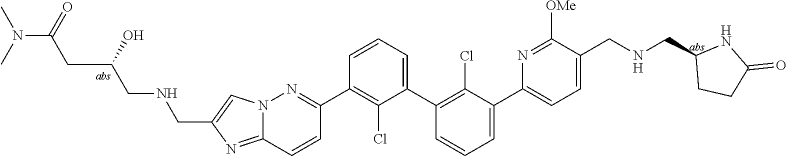 [SMILES: COc1nc(-c2cccc(-c3cccc(-c4ccc5nc(CNC[C@@H](O)CC(=O)N(C)C)cn5n4)c3Cl)c2Cl)ccc1CNC[C@@H]1CCC(=O)N1]

### tert-Butyl N-[[6-[2-chloro-3-[2-chloro-3-[2-[[[(2S)-4-(dimethylamino)-2-hydroxy-4-oxo-butyl]amino]methyl]imidazo[1,2-b]pyridazin-6-yl]phenyl]phenyl]-2-methoxy-3-pyridyl]methyl]-N-[[(2S)-5-oxopyrrolidin-2-yl]methyl]carbamate

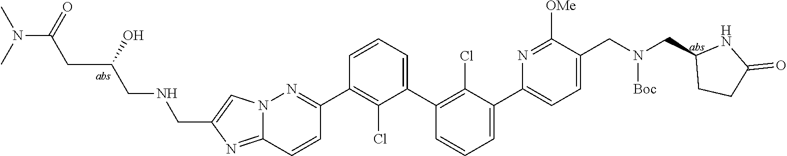 [SMILES: COc1nc(-c2cccc(-c3cccc(-c4ccc5nc(CNC[C@@H](O)CC(=O)N(C)C)cn5n4)c3Cl)c2Cl)ccc1CN(C[C@@H]1CCC(=O)N1)C(=O)OC(C)(C)C]

A mixture of tert-butyl N-[[6-[2-chloro-3-[2-chloro-3-(2-formylimidazo[1,2-b]pyridazin-6-yl)phenyl]phenyl]-2-methoxy-3-pyridyl]methyl]-N-[[(2S)-5-oxopyrrolidin-2-yl]methyl]carbamate (25 mg, 0.04 mmol), (3S)-4-amino-3-hydroxy-N,N-dimethyl-butanamide (10 mg, 0.07 mmol) and acetic acid (2 uL, 2.1 mg, 0.04 mmol) in MeOH/THF (2:1) was stirred for 1 hour at rt. NaBH₃CN (4.5 mg, 0.07 mmol) was added, the mixture was stirred for 1 hour, quenched with water, and purified by reversed phase chromatography to give tert-butyl N-[[6-[2-chloro-3-[2-chloro-3-[2-[[[(2S)-4-(dimethylamino)-2-hydroxy-4-oxo-butyl]amino]methyl]imidazo[1,2-b]pyridazin-6-yl]phenyl]phenyl]-2-methoxy-3-pyridyl]methyl]-N-[[(2S)-5-oxopyrrolidin-2-yl]methyl]carbamate (25 mg, 84.3%). MS: m/z found 831.3 [M+H]⁺.

### (3S)-4-[[6-[2-Chloro-3-[2-chloro-3-[6-methoxy-5-[[[(2S)-5-oxopyrrolidin-2-yl]methylamino]methyl]-2-pyridyl]phenyl]phenyl]imidazo[1,2-b]pyridazin-2-yl]methylamino]-3-hydroxy-N,N-dimethyl-butanamide

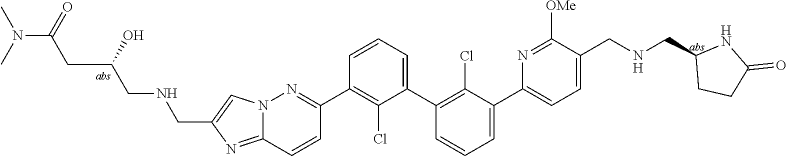 [SMILES: COc1nc(-c2cccc(-c3cccc(-c4ccc5nc(CNC[C@@H](O)CC(=O)N(C)C)cn5n4)c3Cl)c2Cl)ccc1CNC[C@@H]1CCC(=O)N1]

A solution of tert-butyl N-[[6-[2-chloro-3-[2-chloro-3-[2-[[[(2S)-4-(dimethylamino)-2-hydroxy-4-oxo-butyl]amino]methyl]imidazo[1,2-b]pyridazin-6-yl]phenyl]phenyl]-2-methoxy-3-pyridyl]methyl]-N-[[(2S)-5-oxopyrrolidin-2-yl]methyl]carbamate (20 mg, 0.02 mmol) in DCM/TFA (2:1) was stirred for 30 minutes, concentrated and purified by reversed phase chromatography to give (3S)-4-[[6-[2-chloro-3-[2-chloro-3-[6-methoxy-5-[[[(2S)-5-oxopyrrolidin-2-yl]methylamino]methyl]-2-pyridyl]phenyl]phenyl]imidazo[1,2-b]pyridazin-2-yl]methylamino]-3-hydroxy-N,N-dimethyl-butanamide (17 mg, 96.6%). MS: m/z found 731.3 [M+H]⁺. ¹H NMR (400 MHz, Methanol-d₄): δ 8.23 (s, 1H), 8.05 (d, J=9.5 Hz, 1H), 7.74 (d, J=7.5 Hz, 1H), 7.67 (ddd, J=6.8, 4.7, 1.7 Hz, 2H), 7.59 (t, J=7.6 Hz, 1H), 7.54-7.48 (m, 3H), 7.40 (dd, J=7.6, 1.7 Hz, 1H), 7.25 (d, J=7.4 Hz, 1H), 4.30-4.20 (m, 1H), 4.17 (d, J=3.6 Hz, 2H), 4.02 (s, 3H), 3.89-3.78 (m, 3H), 3.07 (s, 3H), 2.99-2.95 (m, 1H), 2.93 (s, 3H), 2.83 (dd, J=12.3, 8.4 Hz, 1H), 2.76-2.68 (m, 2H), 2.68-2.58 (m, 1H), 2.58-2.50 (m, 1H), 2.38-2.25 (m, 3H), 1.87-1.77 (m, 1H).

### Example 356: (5S)-5-[[[6-[2-Chloro-3-[2-chloro-3-[2-[[[(2S)-5-oxopyrrolidin-2-yl]methylamino]methyl]imidazo[1,2-a]pyridin-6-yl]phenyl]phenyl]-2-methoxy-3-pyridyl]methylamino]methyl]pyrrolidin-2-one

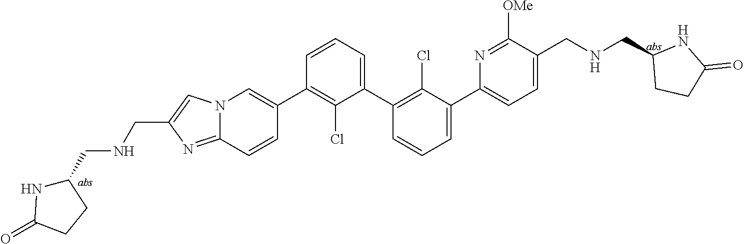 [SMILES: COc1nc(-c2cccc(-c3cccc(-c4ccc5nc(CNC[C@@H]6CCC(=O)N6)cn5c4)c3Cl)c2Cl)ccc1CNC[C@@H]1CCC(=O)N1]

### 6-[2-Chloro-3-[2-chloro-3-(5-formyl-6-methoxy-2-pyridyl)phenyl]phenyl]imidazo[1,2-a]pyridine-2-carbaldehyde

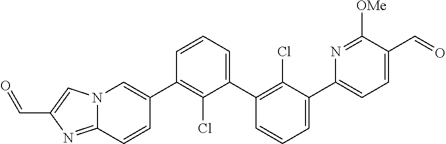 [SMILES: COc1nc(-c2cccc(-c3cccc(-c4ccc5nc(C=O)cn5c4)c3Cl)c2Cl)ccc1C=O]

To a solution of 6-bromoimidazo[1,2-a]pyridine-2-carbaldehyde (20 mg, 0.09 mmol), 6-[2-chloro-3-[2-chloro-3-(4,4,5,5-tetramethyl-1,3,2-dioxaborolan-2-yl)phenyl]phenyl]-2-methoxy-pyridine-3-carbaldehyde (43 mg, 0.09 mmol) and potassium carbonate (37 mg, 0.27 mmol) in degassed dioxane/water (6:1) was added Pd(PPh₃)₄ (10 mg, 0.01 mmol) and the mixture was stirred at 90° C. for 2 hours under N₂(g). Water was added and the mixture was extracted into EtOAc three times. The combined organic layers were dried over anhydrous sodium sulfate, concentrated, and purified on silica gel column using EtOAc in hexanes (0 to 20% gradient) to provide 6-[2-chloro-3-[2-chloro-3-(5-formyl-6-methoxy-2-pyridyl)phenyl]phenyl]imidazo[1,2-a]pyridine-2-carbaldehyde (18 mg, 40.3%). MS: m/z found 502.1 [M+H]⁺.

### (5S)-5-[[[6-[2-Chloro-3-[2-chloro-3-[2-[[[(2S)-5-oxopyrrolidin-2-yl]methylamino]methyl]imidazo[1,2-a]pyridin-6-yl]phenyl]phenyl]-2-methoxy-3-pyridyl]methylamino]methyl]pyrrolidin-2-one

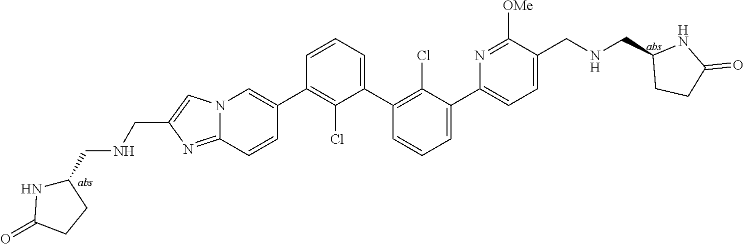 [SMILES: COc1nc(-c2cccc(-c3cccc(-c4ccc5nc(CNC[C@@H]6CCC(=O)N6)cn5c4)c3Cl)c2Cl)ccc1CNC[C@@H]1CCC(=O)N1]

A mixture of 6-[2-chloro-3-[2-chloro-3-(5-formyl-6-methoxy-2-pyridyl)phenyl]phenyl]imidazo[1,2-a]pyridine-2-carbaldehyde (30 mg, 0.06 mmol), (5S)-5-(aminomethyl)pyrrolidin-2-one (27 mg, 0.24 mmol) and acetic acid (6.8 uL, 7 mg, 0.12 mmol) in MeOH/THF (1:1) was stirred for 1.5 hour at rt. NaBH₃CN (15 mg, 0.24 mmol) was added, the mixture was stirred for additional 1 h, quenched with water and directly purified by reversed phase chromatography to give (5S)-5-[[[6-[2-chloro-3-[2-chloro-3-[2-[[[(2S)-5-oxopyrrolidin-2-yl]methylamino]methyl]imidazo[1,2-a]pyridin-6-yl]phenyl]phenyl]-2-methoxy-3-pyridyl]methylamino]methyl]pyrrolidin-2-one (32 mg, 76.7%). MS: m/z found 698.2 [M+H]⁺. ¹H NMR (400 MHz, Methanol-d₄): δ 8.56 (dd, J=1.8, 1.0 Hz, 1H), 7.90 (s, 1H), 7.78 (d, J=7.5 Hz, 1H), 7.65 (dd, J=7.8, 1.7 Hz, 1H), 7.59 (d, J=9.3 Hz, 1H), 7.56-7.48 (m, 3H), 7.46 (dd, J=9.4, 1.7 Hz, 1H), 7.42 (dd, J=6.5, 2.9 Hz, 1H), 7.39 (dd, J=7.6, 1.7 Hz, 1H), 7.28 (d, J=7.4 Hz, 1H), 4.13-4.05 (m, 2H), 4.04 (s, 3H), 3.97 (d, J=3.7 Hz, 2H), 3.94-3.86 (m, 2H), 2.93-2.80 (m, 4H), 2.39-2.26 (m, 6H), 1.92-1.78 (m, 2H).

### Example 357: (5S)-5-[[[6-[2-Chloro-3-[2-chloro-3-[2-[[[(2S)-5-oxopyrrolidin-2-yl]methylamino]methyl]imidazo[1,2-a]pyridin-7-yl]phenyl]phenyl]-2-methoxy-3-pyridyl]methylamino]methyl]pyrrolidin-2-one

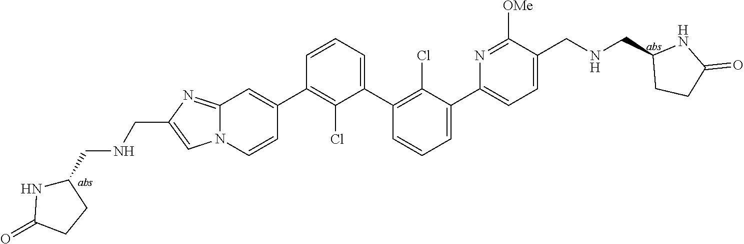 [SMILES: COc1nc(-c2cccc(-c3cccc(-c4ccn5cc(CNC[C@@H]6CCC(=O)N6)nc5c4)c3Cl)c2Cl)ccc1CNC[C@@H]1CCC(=O)N1]

### 7-[2-Chloro-3-[2-chloro-3-(5-formyl-6-methoxy-2-pyridyl)phenyl]phenyl]imidazo[1,2-a]pyridine-2-carbaldehyde

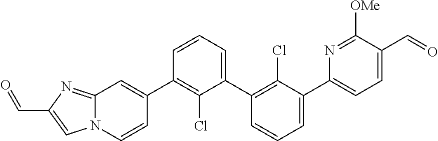 [SMILES: COc1nc(-c2cccc(-c3cccc(-c4ccn5cc(C=O)nc5c4)c3Cl)c2Cl)ccc1C=O]

To a solution of 7-bromoimidazo[1,2-a]pyridine-2-carbaldehyde (20 mg, 0.09 mmol), 6-[2-chloro-3-[2-chloro-3-(4,4,5,5-tetramethyl-1,3,2-dioxaborolan-2-yl)phenyl]phenyl]-2-methoxy-pyridine-3-carbaldehyde (43 mg, 0.09 mmol) and potassium carbonate (37 mg, 0.27 mmol) in degassed dioxane/water (6:1) was added Pd(PPh₃)₄ (10 mg, 0.01 mmol) and the mixture was stirred at 90° C. for 2 hours under N₂(g). Water was added and the mixture was extracted into EtOAc three times. The combined organic layers were dried over anhydrous sodium sulfate, concentrated, and purified on silica gel column using EtOAc in hexanes (0 to 20% gradient) to provide 7-[2-chloro-3-[2-chloro-3-(5-formyl-6-methoxy-2-pyridyl)phenyl]phenyl]imidazo[1,2-a]pyridine-2-carbaldehyde (20 mg, 44.8%). MS: m/z found 502.1 [M+H]⁺.

### (5S)-5-[[[6-[2-Chloro-3-[2-chloro-3-[2-[[[(2S)-5-oxopyrrolidin-2-yl]methylamino]methyl]imidazo[1,2-a]pyridin-7-yl]phenyl]phenyl]-2-methoxy-3-pyridyl]methylamino]methyl]pyrrolidin-2-one

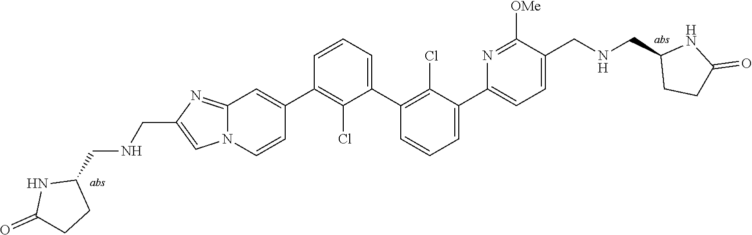 [SMILES: COc1nc(-c2cccc(-c3cccc(-c4ccn5cc(CNC[C@@H]6CCC(=O)N6)nc5c4)c3Cl)c2Cl)ccc1CNC[C@@H]1CCC(=O)N1]

A mixture of 7-[2-chloro-3-[2-chloro-3-(5-formyl-6-methoxy-2-pyridyl)phenyl]phenyl]imidazo[1,2-a]pyridine-2-carbaldehyde (30 mg, 0.06 mmol), (5S)-5-(aminomethyl)pyrrolidin-2-one (27 mg, 0.24 mmol) and acetic acid (6.8 uL, 7 mg, 0.12 mmol) in MeOH/THF (1:1) was stirred for 1.5 hour at rt. NaBH₃CN (15 mg, 0.24 mmol) was added, the mixture was stirred for additional 1 h, quenched with water and directly purified by reversed phase chromatography to give (5S)-5-[[[6-[2-chloro-3-[2-chloro-3-[2-[[[(2S)-5-oxopyrrolidin-2-yl]methylamino]methyl]imidazo[1,2-a]pyridin-7-yl]phenyl]phenyl]-2-methoxy-3-pyridyl]methylamino]methyl]pyrrolidin-2-one (32 mg, 76.7%). MS: m/z found 698.2 [M+H]⁺. ¹H NMR (400 MHz, Methanol-d₄): δ 8.47 (dd, J=7.0, 1.0 Hz, 1H), 7.87 (s, 1H), 7.76 (d, J=7.5 Hz, 1H), 7.65 (dd, J=7.7, 1.7 Hz, 1H), 7.57 (dd, J=1.7, 0.9 Hz, 1H), 7.56-7.47 (m, 3H), 7.44-7.37 (m, 2H), 7.27 (d, J=7.4 Hz, 1H), 7.07 (dd, J=7.0, 1.7 Hz, 1H), 4.08-3.98 (m, 2H), 4.03 (s, 3H), 3.96-3.89 (m, 2H), 3.89-3.83 (m, 2H), 2.88-2.73 (m, 4H), 2.39-2.24 (m, 6H), 1.89-1.77 (m, 2H).

### Example 369: (5S)-5-[[[2-[2-Chloro-3-[2-chloro-3-[6-methoxy-5-[[[(2S)-5-oxopyrrolidin-2-yl]methylamino]methyl]-2-pyridyl]phenyl]phenyl]-6-quinolyl]methylamino]methyl]pyrrolidin-2-one

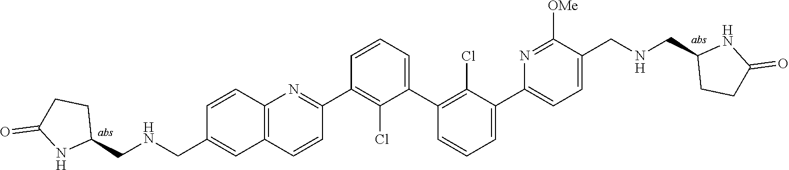 [SMILES: COc1nc(-c2cccc(-c3cccc(-c4ccc5cc(CNC[C@@H]6CCC(=O)N6)ccc5n4)c3Cl)c2Cl)ccc1CNC[C@@H]1CCC(=O)N1]

### 2-[2-Chloro-3-[2-chloro-3-(5-formyl-6-methoxy-2-pyridyl)phenyl]phenyl]quinoline-6-carbaldehyde

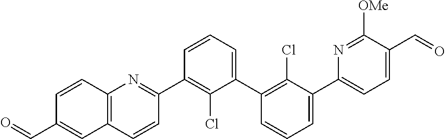 [SMILES: COc1nc(-c2cccc(-c3cccc(-c4ccc5cc(C=O)ccc5n4)c3Cl)c2Cl)ccc1C=O]

To a solution of 2-chloroquinoline-6-carbaldehyde (20 mg, 0.10 mmol), 6-[2-chloro-3-[2-chloro-3-(4,4,5,5-tetramethyl-1,3,2-dioxaborolan-2-yl)phenyl]phenyl]-2-methoxy-pyridine-3-carbaldehyde (51 mg, 0.10 mmol) and potassium carbonate (43 mg, 0.31 mmol) in degassed dioxane/water (6:1) was added Pd(PPh₃)₄ (12 mg, 0.01 mmol) and the mixture was stirred at 90° C. for 2 hours under N₂(g). Water was added and the mixture was extracted into EtOAc three times. The combined organic layers were dried over anhydrous sodium sulfate, concentrated, and purified on silica gel column using EtOAc in hexanes (0 to 20% gradient) to provide 2-[2-chloro-3-[2-chloro-3-(5-formyl-6-methoxy-2-pyridyl)phenyl]phenyl]quinoline-6-carbaldehyde (35 mg, 65.3%). MS: m/z found 513.1 [M+H]⁺.

### (5S)-5-[[[2-[2-Chloro-3-[2-chloro-3-[6-methoxy-5-[[[(2S)-5-oxopyrrolidin-2-yl]methylamino]methyl]-2-pyridyl]phenyl]phenyl]-6-quinolyl]methylamino]methyl]pyrrolidin-2-one

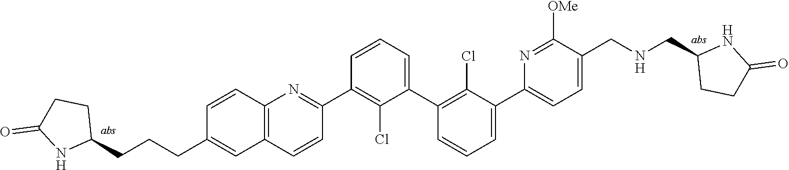 [SMILES: COc1nc(-c2cccc(-c3cccc(-c4ccc5cc(CCC[C@@H]6CCC(=O)N6)ccc5n4)c3Cl)c2Cl)ccc1CNC[C@@H]1CCC(=O)N1]

A mixture of 2-[2-chloro-3-[2-chloro-3-(5-formyl-6-methoxy-2-pyridyl)phenyl]phenyl]quinoline-6-carbaldehyde (30 mg, 0.06 mmol), (5S)-5-(aminomethyl)pyrrolidin-2-one (27 mg, 0.23 mmol) and acetic acid (6.7 uL, 7. mg, 0.12 mmol) in MeOH/THF (1:1) was stirred for 1.5 hour at rt. NaBH₃CN (15 mg, 0.23 mmol) was added, the mixture was stirred for additional 1 h, quenched with water and directly purified by reversed phase chromatography to give (5S)-5-[[[2-[2-chloro-3-[2-chloro-3-[6-methoxy-5-[[[(2S)-5-oxopyrrolidin-2-yl]methylamino]methyl]-2-pyridyl]phenyl]phenyl]-6-quinolyl]methylamino]methyl]pyrrolidin-2-one (36 mg, 86.8%). MS: m/z found 709.2 [M+H]⁺. ¹H NMR (400 MHz, Methanol-d₄): δ 8.43 (d, J=8.5 Hz, 1H), 8.10 (d, J=8.7 Hz, 1H), 8.00 (d, J=1.8 Hz, 1H), 7.86 (dd, J=8.7, 2.0 Hz, 1H), 7.77 (dd, J=8.0, 4.6 Hz, 2H), 7.66 (ddd, J=7.6, 5.7, 1.8 Hz, 2H), 7.59 (t, J=7.6 Hz, 1H), 7.55-7.47 (m, 2H), 7.42 (dd, J=7.6, 1.8 Hz, 1H), 7.28 (d, J=7.5 Hz, 1H), 4.16-4.06 (m, 2H), 4.04 (s, 3H), 4.00-3.92 (m, 2H), 3.91-3.83 (m, 2H), 2.88-2.74 (m, 4H), 2.38-2.25 (m, 6H), 1.89-1.78 (m, 2H).

### Example 370: (5S)-5-[[[3-[2-Chloro-3-[2-chloro-3-[6-methoxy-5-[[[(2S)-5-oxopyrrolidin-2-yl]methylamino]methyl]-2-pyridyl]phenyl]phenyl]-7-quinolyl]methylamino]methyl]pyrrolidin-2-one

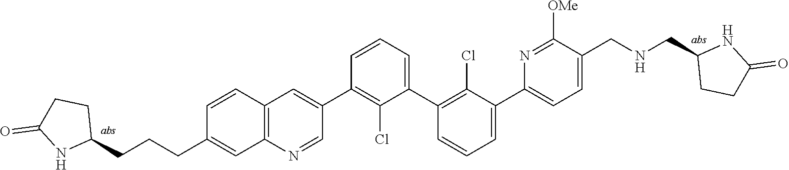 [SMILES: COc1nc(-c2cccc(-c3cccc(-c4cnc5cc(CCC[C@@H]6CCC(=O)N6)ccc5c4)c3Cl)c2Cl)ccc1CNC[C@@H]1CCC(=O)N1]

### 3-[2-Chloro-3-[2-chloro-3-(5-formyl-6-methoxy-2-pyridyl)phenyl]phenyl]quinoline-7-carbaldehyde

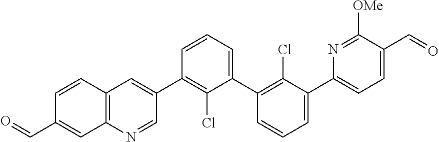 [SMILES: COc1nc(-c2cccc(-c3cccc(-c4cnc5cc(C=O)ccc5c4)c3Cl)c2Cl)ccc1C=O]

To a solution of 3-bromoquinoline-7-carbaldehyde (25 mg, 0.11 mmol), 6-[2-chloro-3-[2-chloro-3-(4,4,5,5-tetramethyl-1,3,2-dioxaborolan-2-yl)phenyl]phenyl]-2-methoxy-pyridine-3-carbaldehyde (51 mg, 0.11 mmol) and potassium carbonate (44 mg, 0.32 mmol) in degassed dioxane/water (6:1) was added Pd(PPh₃)₄ (12 mg, 0.01 mmol) and the mixture was stirred at 90° for 2 hours under N₂(g). Water was added and the mixture was extracted into EtOAc three times. The combined organic layers were dried over anhydrous sodium sulfate, concentrated, and purified on silica gel column using EtOAc in hexanes (0 to 20% gradient) to provide 3-[2-chloro-3-[2-chloro-3-(5-formyl-6-methoxy-2-pyridyl)phenyl]phenyl]quinoline-7-carbaldehyde (36 mg, 66.2%). MS: m/z found 513.1 [M+H]⁺.

### (5S)-5-[[[3-[2-Chloro-3-[2-chloro-3-[6-methoxy-5-[[[(2S)-5-oxopyrrolidin-2-yl]methylamino]methyl]-2-pyridyl]phenyl]phenyl]-7-quinolyl]methylamino]methyl]pyrrolidin-2-one

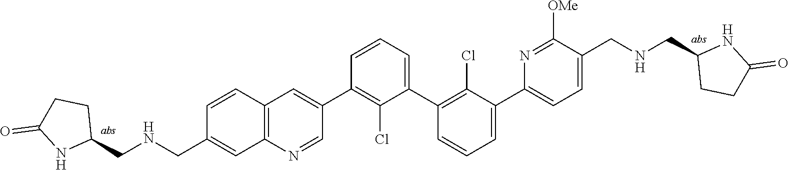 [SMILES: COc1nc(-c2cccc(-c3cccc(-c4cnc5cc(CNC[C@@H]6CCC(=O)N6)ccc5c4)c3Cl)c2Cl)ccc1CNC[C@@H]1CCC(=O)N1]

A mixture of 3-[2-chloro-3-[2-chloro-3-(5-formyl-6-methoxy-2-pyridyl)phenyl]phenyl]quinoline-7-carbaldehyde (30 mg, 0.06 mmol), (5S)-5-(aminomethyl)pyrrolidin-2-one (27 mg, 0.23 mmol) and acetic acid (6.7 uL, 7 mg, 0.12 mmol) in MeOH/THF (1:1) was stirred for 1.5 hour at rt. NaBH₃CN (15 mg, 0.23 mmol) was added, the mixture was stirred for additional 1 h, quenched with water and directly purified by reversed phase chromatography to give (5S)-5-[[[3-[2-chloro-3-[2-chloro-3-[6-methoxy-5-[[[(2S)-5-oxopyrrolidin-2-yl]methylamino]methyl]-2-pyridyl]phenyl]phenyl]-7-quinolyl]methylamino]methyl]pyrrolidin-2-one (35 mg, 84.4%). MS: m/z found 709.2 [M+H]⁺. ¹H NMR (400 MHz, Methanol-d₄): δ 8.97 (d, J=2.2 Hz, 1H), 8.46 (d, J=2.2 Hz, 1H), 8.07 (d, J=1.5 Hz, 1H), 8.04 (d, J=8.4 Hz, 1H), 7.77 (d, J=7.5 Hz, 1H), 7.73 (dd, J=8.4, 1.7 Hz, 1H), 7.65 (dd, J=7.7, 1.7 Hz, 1H), 7.63-7.55 (m, 2H), 7.51 (t, J=7.6 Hz, 1H), 7.46 (dd, J=7.2, 2.1 Hz, 1H), 7.41 (dd, J=7.6, 1.7 Hz, 1H), 7.28 (d, J=7.4 Hz, 1H), 4.19-4.06 (m, 2H), 4.04 (s, 3H), 3.98-3.91 (m, 2H), 3.91-3.84 (m, 2H), 2.88-2.70 (m, 4H), 2.39-2.23 (m, 6H), 1.90-1.77 (m, 2H).

### Example 382: (5S)-5-[[[6-[2-chloro-3-[2-chloro-3-[3-chloro-2-[[[(2S)-5-oxopyrrolidin-2-yl]methylamino]methyl]imidazo[1,2-a]pyridin-7-yl]phenyl]phenyl]-2-methoxy-3-pyridyl]methylamino]methyl]pyrrolidin-2-one

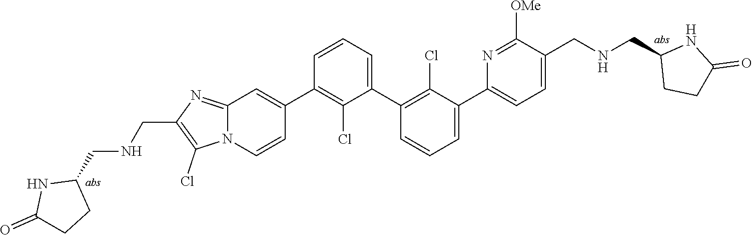 [SMILES: COc1nc(-c2cccc(-c3cccc(-c4ccn5c(Cl)c(CNC[C@@H]6CCC(=O)N6)nc5c4)c3Cl)c2Cl)ccc1CNC[C@@H]1CCC(=O)N1]

### (7-bromoimidazo[1,2-a]pyridin-2-yl)methanol

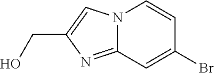 [SMILES: OCc1cn2ccc(Br)cc2n1]

NaBH₄ (20.17 mg, 0.53 mmol) was added to a solution of 7-bromoimidazo[1,2-a]pyridine-2-carbaldehyde (100 mg, 0.44 mmol) in ethanol (2 mL). This mixture was stirred for 1 b (TLC showed full conversion to the product after 30 min stirring at rt), after which brine was added. The resulting mixture was extracted three times with EtOAc, and the combined organic phases were dried over MgSO₄. Evaporation in vacuo provided (7-bromoimidazo[1,2-a]pyridin-2-yl)methanol (98 mg, 97.1%). MS: m/z found 227.0 [M+H]⁺. ¹H NMR (400 MHz, Chloroform-d): δ 7.90 (dd, J=7.1, 0.8 Hz, 1H), 7.69 (dd, J=1.9, 0.9 Hz, 1H), 7.50 (s, 1H), 6.84 (dd, J=7.1, 1.9 Hz, 1H), 4.79 (s, 2H).

### (7-Bromo-3-chloro-imidazo[1,2-a]pyridin-2-yl)methanol

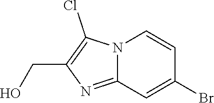 [SMILES: OCc1nc2cc(Br)ccn2c1Cl]

To a suspension of (7-bromoimidazo[1,2-a]pyridin-2-yl)methanol (50 mg, 0.22 mmol) in DCM was added NCS (32 mg, 0.24 mmol) in one portion. After 2 hours, the reaction mixture was concentrated under reduced pressure and the residue was purified directly by silica gel chromatography to give (7-bromo-3-chloro-imidazo[1,2-a]pyridin-2-yl)methanol (56 mg, 97.2%). MS: m/z found 260.9 [M+H]⁺. ¹H NMR (400 MHz, Chloroform-d): δ 7.92 (dd, J=7.3, 0.8 Hz, 1H), 7.89 (m, 1H), 7.06 (dd, J=7.2, 1.9 Hz, 1H), 4.81 (s, H).

### 7-Bromo-3-chloro-imidazo[1,2-a]pyridine-2-carbaldehyde

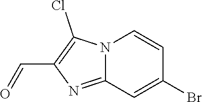 [SMILES: O=Cc1nc2cc(Br)ccn2c1Cl]

To a suspension of (7-bromo-3-chloro-imidazo[1,2-a]pyridin-2-yl)methanol (55 mg, 0.21 mmol) in anhydrous DCM under nitrogen atmosphere was added Dess-Martin periodinane (116 mg, 0.27 mmol). After 2 h stirring, saturated solution of NaHCO₃an Na₂S₂; was added to the reaction mixture. Upon vigorously stirring for a while, layers were separated, and organic layer was washed with sat NaHCO₃ solution again. Aqueous layer was extracted into DCM as well. The combined organic layers were dried over anhydrous sodium sulfate and concentrated to give 7-bromo-3-chloro-imidazo[1,2-a]pyridine-2-carbaldehyde (52 mg, 95.3%). MS: m/z found 258.9 [M+H]⁺. 1H NMR (400 MHz, Chloroform-d): δ 10.20 (s, 1H), 8.03 (dd, J=7.4, 0.9 Hz, 1H), 7.88 (dd, J=1.9, 0.9 Hz, 1H), 7.15 (dd, J=7.3, 1.8 Hz, 1H).

### 3-Chloro-7-[2-chloro-3-[2-chloro-3-(5-formyl-6-methoxy-2-pyridyl)phenyl]phenyl]imidazo[1,2-a]pyridine-2-carbaldehyde

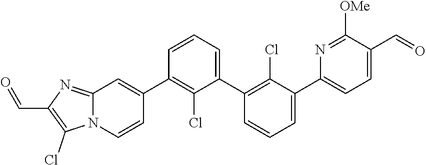 [SMILES: COc1nc(-c2cccc(-c3cccc(-c4ccn5c(Cl)c(C=O)nc5c4)c3Cl)c2Cl)ccc1C=O]

To a solution of 7-bromo-3-chloro-imidazo[1,2-a]pyridine-2-carbaldehyde (20 mg, 0.08 mmol), 6-[2-chloro-3-[2-chloro-3-(4,4,5,5-tetramethyl-1,3,2-dioxaborolan-2-yl)phenyl]phenyl]-2-methoxy-pyridine-3-carbaldehyde (37.32 mg, 0.08 mmol) and potassium carbonate (32 mg, 0.23 mmol) in degassed dioxane/water (6:1) was added Pd(PPh₃)₄ (8.91 mg, 0.01 mmol) and the mixture was stirred at 90° C. for 2 hours under N₂(g). Water was added and the mixture was extracted into EtOAc three times. The combined organic layers were dried over anhydrous sodium sulfate, concentrated, and purified on silica gel column using EtOAc in hexanes (0 to 20% gradient) to provide 3-chloro-7-[2-chloro-3-[2-chloro-3-(5-formyl-6-methoxy-2-pyridyl)phenyl]phenyl]imidazo[1,2-a]pyridine-2-carbaldehyde (36 mg, 87.0%). MS: m/z found 536.0 [M+H]⁺.

### (5S)-5-[[[6-[2-Chloro-3-[2-chloro-3-[3-chloro-2-[[[(2S)-5-oxopyrrolidin-2-yl]methylamino]methyl]imidazo[1,2-a]pyridin-7-yl]phenyl]phenyl]-2-methoxy-3-pyridyl]methylamino]methyl]pyrrolidin-2-one

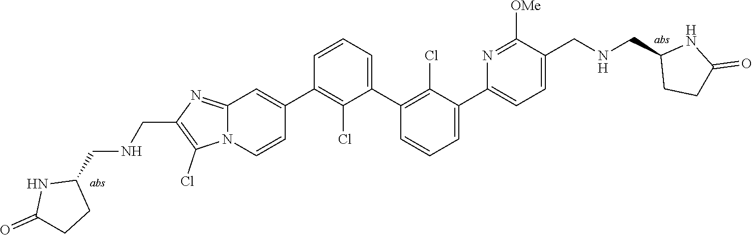 [SMILES: COc1nc(-c2cccc(-c3cccc(-c4ccn5c(Cl)c(CNC[C@@H]6CCC(=O)N6)nc5c4)c3Cl)c2Cl)ccc1CNC[C@@H]1CCC(=O)N1]

A mixture of 3-chloro-7-[2-chloro-3-[2-chloro-3-(5-formyl-6-methoxy-2-pyridyl)phenyl]phenyl]imidazo[1,2-a]pyridine-2-carbaldehyde (25 mg, 0.05 mmol), (5S)-5-(aminomethyl)pyrrolidin-2-one (21 mg, 0.19 mmol) and acetic acid (5 uL, 5.6 mg, 0.09 mmol) in MeOH/THF (1:1) was stirred for 1.5 hour at rt. NaBH₃CN (12 mg, 0.19 mmol) was added, the mixture was stirred for additional 1 h, quenched with water and directly purified by reversed phase chromatography to give (5S)-5-[[[6-[2-chloro-3-[2-chloro-3-[3-chloro-2-[[[(2S)-5-oxopyrrolidin-2-yl]methylamino]methyl]imidazo[1,2-a]pyridin-7-yl]phenyl]phenyl]-2-methoxy-3-pyridyl]methylamino]methyl]pyrrolidin-2-one (29 mg, 84.9%). MS: m/z found 732.2 [M+H]⁺. ¹H NMR (400 MHz, Methanol-d₄): δ 8.37 (dd, J=7.1, 1.0 Hz, 1H), 7.78 (d, J=7.5 Hz, 1H), 7.68-7.62 (m, 2H), 7.58-7.48 (m, 3H), 7.43 (dd, J=6.4, 2.9 Hz, 1H), 7.40 (dd, J=7.6, 1.7 Hz, 1H), 7.28 (d, J=7.4 Hz, 1H), 7.26 (dd, J=7.1, 1.7 Hz, 1H), 4.09-4.02 (m, 2H), 4.04 (s, 3H), 4.01-3.97 (m, 2H), 3.96-3.87 (m, 1H), 3.87-3.80 (m, 1H), 2.93-2.70 (m, 4H), 2.42-2.21 (m, 6H), 1.90-1.75 (m, 2H).

### Example 383: 2-[[6-[2-Chloro-3-[2-chloro-3-[3-chloro-2-[(2-hydroxyethylamino)methyl]imidazo[1,2-a]pyridin-7-yl]phenyl]phenyl]-2-methoxy-3-pyridyl]methylamino]etanol

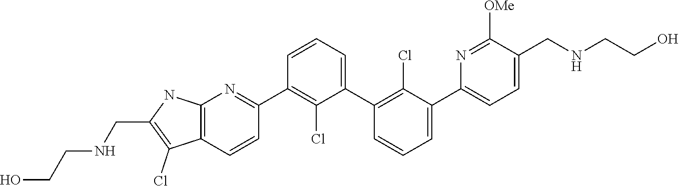 [SMILES: COc1nc(-c2cccc(-c3cccc(-c4ccc5c(Cl)c(CNCCO)[nH]c5n4)c3Cl)c2Cl)ccc1CNCCO]

A mixture of 3-chloro-7-[2-chloro-3-[2-chloro-3-(5-formyl-6-methoxy-2-pyridyl)phenyl]phenyl]imidazo[1,2-a]pyridine-2-carbaldehyde (10. mg, 0.02 mmol), 2-aminoethanol (4.6 mg, 0.07 mmol) and acetic acid (2.1 uL, 2.2 mg, 0.04 mmol) in MeOH/THF (1:1) was stirred for 1.5 hour at rt. NaBH₃CN (5 mg, 0.07 mmol) was added, the mixture was stirred for additional 1 h, quenched with water and directly purified by reversed phase chromatography to give 2-[[6-[2-chloro-3-[2-chloro-3-[3-chloro-2-[(2-hydroxyethylamino)methyl]imidazo[1,2-a]pyridin-7-yl]phenyl]phenyl]-2-methoxy-3-pyridyl]methylamino]ethanol (9 mg, 77.1%). MS: m/z found 626.1 [M+H]⁺. ¹H NMR (400 MHz, Methanol-d₄): δ 8.38 (dd, J=7.1, 1.0 Hz, 1H), 7.78 (d, J=7.5 Hz, 1H), 7.65 (ddd, J=4.3, 3.4, 1.8 Hz, 2H), 7.58-7.49 (m, 3H), 7.44 (dd, J=6.5, 2.8 Hz, 1H), 7.40 (dd, J=7.5, 1.7 Hz, 1H), 7.29 (d, J=7.4 Hz, 1H), 7.26 (dd, J=7.1, 1.7 Hz, 1H), 4.06 (s, 2H), 4.05 (s, 3H), 4.02 (s, 2H), 3.77-3.74 (m, 2H), 3.73-3.69 (m, 2H), 2.93 (t, J=5.4 Hz, 2H), 2.86 (t, J=5.5 Hz, 2H).

### Example 388: (5S)-5-[[[6-[2-Chloro-3-[2-chloro-3-[1-methyl-3-[[[(2S)-5-oxopyrrolidin-2-yl]methylamino]methyl]pyrrolo[2,3-b]pyridin-6-yl]phenyl]phenyl]-2-methoxy-3-pyridyl]methylamino]methyl]pyrrolidin-2-one

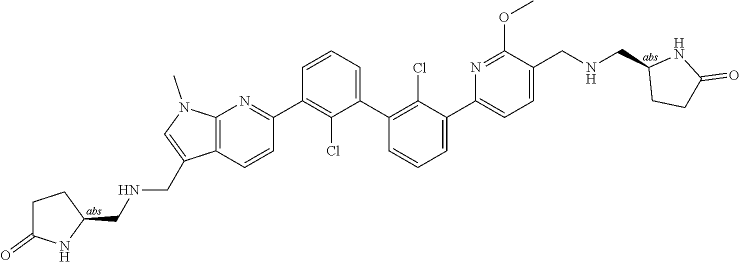 [SMILES: COc1nc(-c2cccc(-c3cccc(-c4ccc5c(CNC[C@@H]6CCC(=O)N6)cn(C)c5n4)c3Cl)c2Cl)ccc1CNC[C@@H]1CCC(=O)N1]

### 6-Chloro-1-methyl-pyrrolo[2,3-b]pyridine-3-carbaldehyde

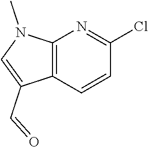

To mixture of 6-chloro-1H-pyrrolo[2,3-b]pyridine-3-carbaldehyde (200 mg, 1.11 mmol) and K₂CO₃ (230 mg, 1.66 mmol) in DMF (3 mL) were added Mel (97 uL, 220 mg, 1.55 mmol). The mixture was stirred at ambient temperature for 2 hrs. After addition of water, the reaction was extracted with EtOAc three times, and the organic layer was washed with a brine solution, and dried over Na₂SO₄. The solvents were removed in vacuo, and the residue was purified by flash column chromatography (EtOAc in hexanes, 0 to 20% gradient) to afford 6-chloro-1-methyl-pyrrolo[2,3-b]pyridine-3-carbaldehyde (212 mg, 98.4%). MS: m/z found 195.0 [M+H]⁺. ¹H NMR (400 MHz, Chloroform-d): δ 9.93 (s, 1H), 8.46 (d, J=8.2 Hz, 1H), 7.81 (s, 1H), 7.26 (d, J=8.2 Hz, 1H), 3.93 (s, 3H).

### 6-[2-Chloro-3-[2-chloro-3-(5-formyl-6-methoxy-2-pyridyl)phenyl]phenyl]-1-methyl-pyrrolo[2,3-b]pyridine-3-carbaldehyde

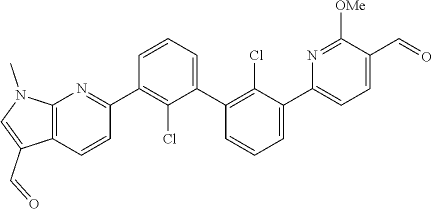 [SMILES: COc1nc(-c2cccc(-c3cccc(-c4ccc5c(C=O)cn(C)c5n4)c3Cl)c2Cl)ccc1C=O]

To a solution of 6-chloro-1-methyl-pyrrolo[2,3-b]pyridine-3-carbaldehyde (15 mg, 0.08 mmol), 6-[2-chloro-3-[2-chloro-3-(4,4,5,5-tetramethyl-1,3,2-dioxaborolan-2-yl)phenyl]phenyl]-2-methoxy-pyridine-3-carbaldehyde (37 mg, 0.08 mmol) and potassium carbonate (31.96 mg, 0.23 mmol) in degassed dioxane/water (6:1) was added Pd(PPh₃)₄ (9 mg, 0.01 mmol) and the mixture was stirred at 90° C. for 2 hours under N₂(g). Water was added and the mixture was extracted into EtOAc three times. The combined organic layers were dried over anhydrous sodium sulfate, concentrated, and purified on silica gel column using EtOAc in hexanes (0 to 20% gradient) to provide 6-[2-chloro-3-[2-chloro-3-(5-formyl-6-methoxy-2-pyridyl)phenyl]phenyl]-1-methyl-pyrrolo[2,3-b]pyridine-3-carbaldehyde (29 mg, 72.9%). MS: m/z found 516.1 [M+H]⁺.

### (5S)-5-[[[6-[2-Chloro-3-[2-chloro-3-[1-methyl-3-[[[(2S)-5-oxopyrrolidin-2-yl]methylamino]methyl]pyrrolo[2,3-b]pyridin-6-yl]phenyl]phenyl]-2-methoxy-3-pyridyl]methylamino]methyl]pyrrolidin-2-one

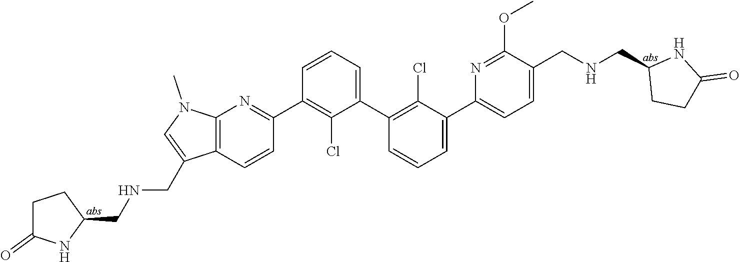 [SMILES: COc1nc(-c2cccc(-c3cccc(-c4ccc5c(CNC[C@@H]6CCC(=O)N6)cn(C)c5n4)c3Cl)c2Cl)ccc1CNC[C@@H]1CCC(=O)N1]

A mixture of 6-[2-chloro-3-[2-chloro-3-(5-formyl-6-methoxy-2-pyridyl)phenyl]phenyl]-1-methyl-pyrrolo[2,3-b]pyridine-3-carbaldehyde (28 mg, 0.05 mmol), (5S)-5-(aminomethyl)pyrrolidin-2-one (25 mg, 0.22 mmol) and acetic acid (6 uL, 6.5 mg, 0.11 mmol) in MeOH/THF (1:1) was stirred for 1.5 hour at rt. NaBH₃CN (14 mg, 0.22 mmol) was added, the mixture was stirred for additional 1 h, quenched with water and directly purified by reversed phase chromatography to give (5S)-5-[[[6-[2-chloro-3-[2-chloro-3-[1-methyl-3-[[[(2S)-5-oxopyrrolidin-2-yl]methylamino]methyl]pyrrolo[2,3-b]pyridin-6-yl]phenyl]phenyl]-2-methoxy-3-pyridyl]methylamino]methyl]pyrrolidin-2-one (30 mg, 77.6%). MS: m/z found 712.3 [M+H]⁺. ¹H NMR (400 MHz, Methanol-d₄): δ 8.21 (d, J=8.1 Hz, 1H), 7.75 (d, J=7.5 Hz, 1H), 7.63 (ddd, J=7.6, 3.6, 1.7 Hz, 2H), 7.55-7.47 (m, 3H), 7.44-7.37 (m, 3H), 7.26 (d, J=7.5 Hz, 1H), 4.27-4.16 (m, 2H), 4.03 (s, 3H), 3.91 (s, 3H), 3.90-3.83 (m, 4H), 3.00-2.88 (m, 2H), 2.83-2.68 (m, 2H), 2.39-2.23 (m, 6H), 1.92-1.75 (m, 2H).

### Example 389: (5S)-5-[[[6-[2-Chloro-3-[2-chloro-3-[3-[[[(2S)-5-oxopyrrolidin-2-yl]methylamino]methyl]imidazo[1,2-a]pyridin-6-yl]phenyl]phenyl]-2-methoxy-3-pyridyl]methylamino]methyl]pyrrolidin-2-one

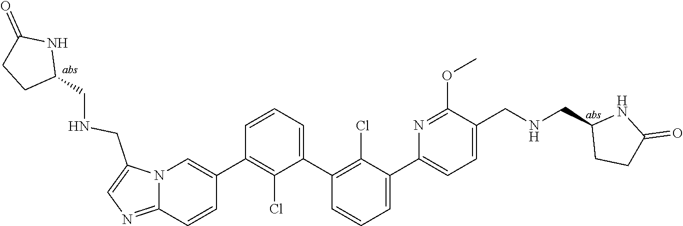 [SMILES: COc1nc(-c2cccc(-c3cccc(-c4ccc5ncc(CNC[C@@H]6CCC(=O)N6)n5c4)c3Cl)c2Cl)ccc1CNC[C@@H]1CCC(=O)N1]

### 6-[2-Chloro-3-[2-chloro-3-(5-formyl-6-methoxy-2-pyridyl)phenyl]phenyl]imidazo[1,2-a]pyridine-3-carbaldehyde

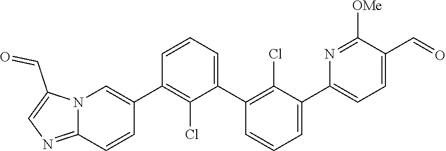 [SMILES: COc1nc(-c2cccc(-c3cccc(-c4ccc5ncc(C=O)n5c4)c3Cl)c2Cl)ccc1C=O]

To a solution of 6-chloroimidazo[1,2-a]pyridine-3-carbaldehyde (10 mg, 0.06 mmol, 6-[2-chloro-3-[2-chloro-3-(4,4,5,5-tetramethyl-1,3,2-dioxaborolan-2-yl)phenyl]phenyl]-2-methoxy-pyridine-3-carbaldehyde (27 mg, 0.06 mmol) and potassium carbonate (23 mg, 0.17 mmol) in degassed dioxane/water (6:1) was added Pd(PPh₃)₄ (6.4 mg, 0.01 mmol) and the mixture was stirred at 90° C. for 2 hours under N₂(g). Water was added and the mixture was extracted into EtOAc three times. The combined organic layers were dried over anhydrous sodium sulfate, concentrated, and purified on silica gel column using EtOAc in hexanes (0 to 20% gradient) to provide 6-[2-chloro-3-[2-chloro-3-(5-formyl-6-methoxy-2-pyridyl)phenyl]phenyl]imidazo[1,2-a]pyridine-3-carbaldehyde (10 mg, 35.9%). MS: m/z found 502.1 [M+H]⁺.

### (5S)-5-[[[6-[2-Chloro-3-[2-chloro-3-[3-[[[(2S)-5-oxopyrrolidin-2-yl]methylamino]methyl]imidazo[1,2-a]pyridin-6-yl]phenyl]phenyl]-2-methoxy-3-pyridyl]methylamino]methyl]pyrrolidin-2-one

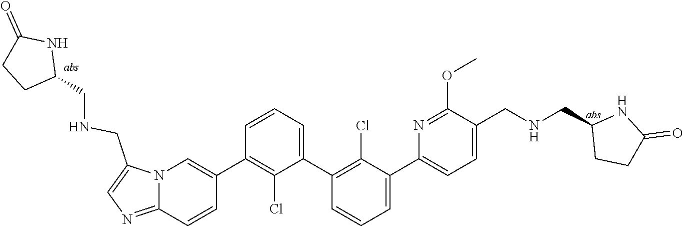 [SMILES: COc1nc(-c2cccc(-c3cccc(-c4ccc5ncc(CNC[C@@H]6CCC(=O)N6)n5c4)c3Cl)c2Cl)ccc1CNC[C@@H]1CCC(=O)N1]

A mixture of 6-[2-chloro-3-[2-chloro-3-(5-formyl-6-methoxy-2-pyridyl)phenyl]phenyl]imidazo[1,2-a]pyridine-3-carbaldehyde (15 mg, 0.03 mmol), (5S)-5-(aminomethyl)pyrrolidin-2-one (13.6 mg, 0.12 mmol) and acetic acid (3.4 uL, 3.6 mg, 0.06 mmol) in MeOH/THF (1:1) was stirred for 1.5 hour at rt. NaBH₃CN (7.5 mg, 0.12 mmol) was added, the mixture was stirred for additional 1 h and quenched with water and directly purified by reversed phase chromatography to give (5S)-5-[[[6-[2-chloro-3-[2-chloro-3-[3-[[[(2S)-5-oxopyrrolidin-2-yl]methylamino]methyl]imidazo[1,2-a]pyridin-6-yl]phenyl]phenyl]-2-methoxy-3-pyridyl]methylamino]methyl]pyrrolidin-2-one (16 mg, 76.7%). MS: m/z found 698.2 [M+H]⁺. ¹H NMR (400 MHz, Methanol-d₄): δ 8.62 (s, 1H), 7.78 (d, J=7.5 Hz, 1H), 7.67-7.57 (m, 4H), 7.56-7.53 (m, 1H), 7.53-7.46 (m, 2H), 7.43 (dd, J=7.6, 1.8 Hz, 1H), 7.39 (dd, J=7.5, 1.7 Hz, 1H), 7.30-7.26 (m, 1H), 4.26-4.16 (m, 2H), 4.05 (s, 3H), 4.01-3.94 (m, 2H), 3.94-3.87 (m, 1H), 3.79-3.72 (m, 1H), 2.93-2.81 (m, 2H), 2.78-2.63 (m, 2H), 2.40-2.30 (m, 3H), 2.30-2.14 (m, 3H), 1.90-1.73 (m, 2H).

### Example 398: (5S)-5-[[[8-Chloro-2-[2-chloro-3-[2-chloro-3-[4-fluoro-6-methoxy-5-[[[(2S)-5-oxopyrrolidin-2-yl]methylamino]methyl]-2-pyridyl]phenyl]phenyl]-5-methoxy-6-quinolyl]methylamino]methyl]pyrrolidin-2-one

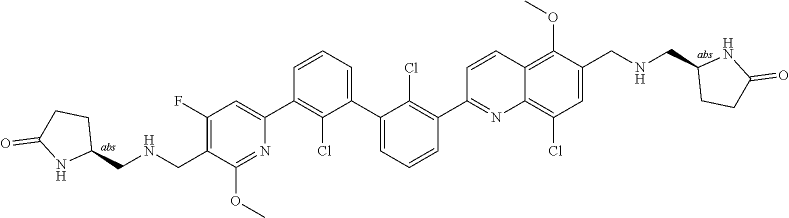 [SMILES: COc1nc(-c2cccc(-c3cccc(-c4ccc5c(OC)c(CNC[C@@H]6CCC(=O)N6)cc(Cl)c5n4)c3Cl)c2Cl)cc(F)c1CNC[C@@H]1CCC(=O)N1]

### 6-(3-Bromo-2-chloro-phenyl)-4-fluoro-2-methoxy-pyridine-3-carbaldehyde

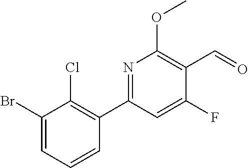 [SMILES: COc1nc(-c2cccc(Br)c2Cl)cc(F)c1C=O]

To a solution of 2-(3-bromo-2-chloro-phenyl)-4,4,5,5-tetramethyl-1,3,2-dioxaborolane (150 mg, 0.47 mmol), 6-chloro-4-fluoro-2-methoxy-pyridine-3-carbaldehyde (90 mg, 0.47 mmol) and potassium carbonate (196 mg, 1.42 mmol) in degassed dioxane/water (6:1) was added Pd(PPh₃)₄ (55 mg, 0.05 mmol) and the mixture was stirred at 90° C. for 2 hours under N₂(g). Water was added and the mixture was extracted into EtOAc three times. The combined organic layers were dried over anhydrous sodium sulfate, concentrated, and purified on silica gel column using EtOAc in hexanes (0 to 20% gradient) to provide 6-(3-bromo-2-chloro-phenyl)-4-fluoro-2-methoxy-pyridine-3-carbaldehyde (125 mg, 76.8%). MS: m/z found 343.9 [M+H]⁺.

### 6-[3-(3-Bromo-2-chloro-phenyl)-2-chloro-phenyl]-4-fluoro-2-methoxy-pyridine-3-carbaldehyde

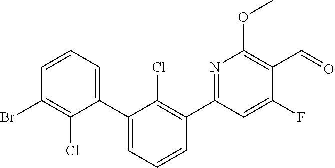 [SMILES: COc1nc(-c2cccc(-c3cccc(Br)c3Cl)c2Cl)cc(F)c1C=O]

To a solution of 2-(3-bromo-2-chloro-phenyl)-4,4,5,5-tetramethyl-1,3,2-dioxaborolane (111 mg, 0.35 mmol), 6-(3-bromo-2-chloro-phenyl)-4-fluoro-2-methoxy-pyridine-3-carbaldehyde (120 mg, 0.35 mmol) and potassium carbonate (144 mg, 1.04 mmol) in degassed dioxane/water (6:1) was added Pd(PPh₃)₄ (40 mg, 0.03 mmol) and the mixture was stirred at 90° C. for 2 hours under N₂(g). Water was added and the mixture was extracted into EtOAc three times. The combined organic layers were dried over anhydrous sodium sulfate, concentrated, and purified on silica gel column using EtOAc in hexanes (0 to 20% gradient) to provide 6-[3-(3-bromo-2-chloro-phenyl)-2-chloro-phenyl]-4-fluoro-2-methoxy-pyridine-3-carbaldehyde (56 mg, 35.3%). MS: m/z found 453.9 [M+H]⁺.

### 6-[2-Chloro-3-[2-chloro-3-(4,4,5,5-tetramethyl-1,3,2-dioxaborolan-2-yl)phenyl]phenyl]-4-fluoro-2-methoxy-pyridine-3-carbaldehyde

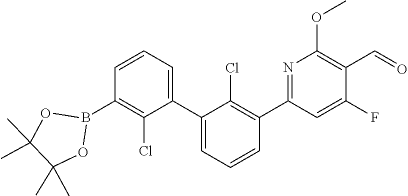 [SMILES: COc1nc(-c2cccc(-c3cccc(B4OC(C)(C)C(C)(C)O4)c3Cl)c2Cl)cc(F)c1C=O]

To a solution of 6-[3-(3-bromo-2-chloro-phenyl)-2-chloro-phenyl]-4-fluoro-2-methoxy-pyridine-3-carbaldehyde (55 mg, 0.12 mmol), 4,4,5,5-tetramethyl-2-(4,4,5,5-tetramethyl-1,3,2-dioxaborolan-2-yl)-1,3,2-dioxaborolane (77 mg, 0.30 mmol) and potassium acetate (36 mg, 0.36 mmol) in degassed dioxane under N₂(g) atmosphere was added Pd(dppf)Cl₂ (9 mg, 0.01 mmol). The reaction mixture was stirred at 85° C. for 4 hours, cooled to room temperature, and concentrated on a rotary evaporator. The crude residue was purified by flash column chromatography (ethyl acetate in hexanes, 0 to 30% gradient) to give 6-[2-chloro-3-[2-chloro-3-(4,4,5,5-tetramethyl-1,3,2-dioxaborolan-2-yl)phenyl]phenyl]-4-fluoro-2-methoxy-pyridine-3-carbaldehyde (52 mg, 85.7%). MS: m/z found 502.1 [M+H]⁺.

### 8-Chloro-2-[2-chloro-3-[2-chloro-3-(4-fluoro-5-formyl-6-methoxy-2-pyridyl)phenyl]phenyl]-5-methoxy-quinoline-6-carbaldehyde

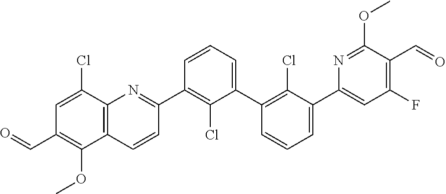 [SMILES: COc1nc(-c2cccc(-c3cccc(-c4ccc5c(OC)c(C=O)cc(Cl)c5n4)c3Cl)c2Cl)cc(F)c1C=O]

To a solution of 2,8-dichloro-5-methoxy-quinoline-6-carbaldehyde (20 mg, 0.08 mmol), 6-[2-chloro-3-[2-chloro-3-(4,4,5,5-tetramethyl-1,3,2-dioxaborolan-2-yl)phenyl]phenyl]-4-fluoro-2-methoxy-pyridine-3-carbaldehyde (39 mg, 0.08 mmol) and potassium carbonate (32 mg, 0.23 mmol) in degassed dioxane/water (6:1) was added Pd(PPh₃)₄ (9 mg, 0.01 mmol) and the mixture was stirred at 90° C. for 2 hours under N₂(g). Water was added and the mixture was extracted into EtOAc three times. The combined organic layers were dried over anhydrous sodium sulfate, concentrated, and purified on silica gel column using EtOAc in hexanes (0 to 40% gradient) to provide 8-chloro-2-[2-chloro-3-[2-chloro-3-(4-fluoro-5-formyl-6-methoxy-2-pyridyl)phenyl]phenyl]-5-methoxy-quinoline-6-carbaldehyde (35 mg, 75.2%). MS: m/z found 595.0 [M+H]⁺.

### (5S)-5-[[[8-chloro-2-[2-Chloro-3-[2-chloro-3-[4-fluoro-6-methoxy-5-[[[(2S)-5-oxopyrrolidin-2-yl]methylamino]methyl]-2-pyridyl]phenyl]phenyl]-5-methoxy-6-quinolyl]methylamino]methyl]pyrrolidin-2-one

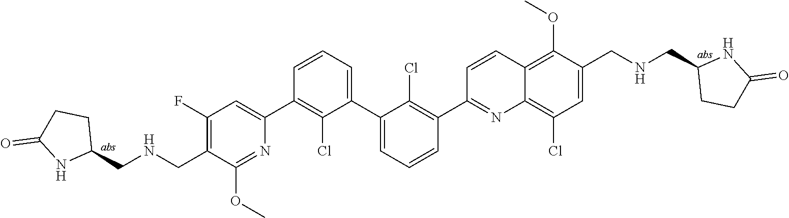 [SMILES: COc1nc(-c2cccc(-c3cccc(-c4ccc5c(OC)c(CNC[C@@H]6CCC(=O)N6)cc(Cl)c5n4)c3Cl)c2Cl)cc(F)c1CNC[C@@H]1CCC(=O)N1]

A mixture of 8-chloro-2-[2-chloro-3-[2-chloro-3-(4-fluoro-5-formyl-6-methoxy-2-pyridyl)phenyl]phenyl]-5-methoxy-quinoline-6-carbaldehyde (25 mg, 0.04 mmol), (5S)-5-(aminomethyl)pyrrolidin-2-one (19 mg, 0.17 mmol) and acetic acid (4.8 uL, 5 mg, 0.08 mmol) in MeOH/THF (1:1) was stirred for 1.5 hour at rt. NaBH₃CN (11 mg, 0.17 mmol) was added, the mixture was stirred for additional 1 h, quenched with water and directly purified by reversed phase chromatography to give (5S)-5-[[[8-chloro-2-[2-chloro-3-[2-chloro-3-[4-fluoro-6-methoxy-5-[[[(2S)-5-oxopyrrolidin-2-yl]methylamino]methyl]-2-pyridyl]phenyl]phenyl]-5-methoxy-6-quinolyl]methylamino]methyl]pyrrolidin-2-one (26 mg, 78.2%). MS: m/z found 791.2 [M+H]⁺. ¹H NMR (400 MHz, Methanol-d₄): δ 8.63 (d, J=8.7 Hz, 1H), 8.04 (s, 1H), 7.93 (d, J=8.7 Hz, 1H), 7.76 (dd, J=7.6, 1.7 Hz, 1H), 7.68 (dd, J=7.6, 1.7 Hz, 1H), 7.59 (t, J=7.6 Hz, 1H), 7.53 (t, J=7.7 Hz, 1H), 7.49 (dd, J=7.7, 1.7 Hz, 1H), 7.46 (dd, J=7.6, 1.7 Hz, 1H), 7.14 (d, J=9.2 Hz, 1H), 4.06 (s, 3H), 4.04 (s, 2H), 4.01 (s, 3H), 3.89 (s, 2H), 3.86-3.79 (m, 2H), 2.77-2.59 (m, 4H), 2.38-2.20 (m, 6H), 1.89-1.73 (m, 2H).

### Example 411: (5S)-5-[[[6-[2-Chloro-3-[2-chloro-3-[3-[[[(2S)-2-hydroxypropyl]amino]methyl]-1-methyl-pyrrolo[2,3-b]pyridin-6-yl]phenyl]phenyl]-2-methoxy-3-pyridyl]methylamino]methyl]pyrrolidin-2-one

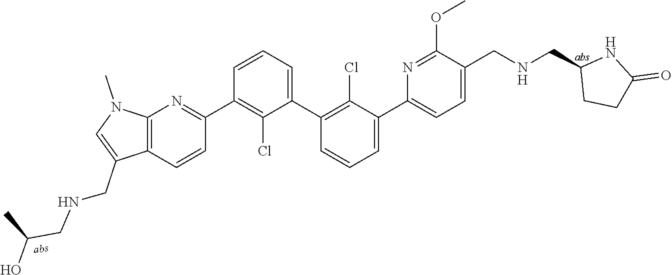 [SMILES: COc1nc(-c2cccc(-c3cccc(-c4ccc5c(CNC[C@H](C)O)cn(C)c5n4)c3Cl)c2Cl)ccc1CNC[C@@H]1CCC(=O)N1]

### tert-Butyl N-[[6-[2-chloro-3-[2-chloro-3-(3-formyl-1-methyl-pyrrolo[2,3-b]pyridin-6-yl)phenyl]phenyl]-2-methoxy-3-pyridyl]methyl]-N-[[(2S)-5-oxopyrrolidin-2-yl]methyl]carbamate

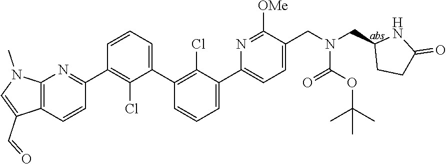 [SMILES: COc1nc(-c2cccc(-c3cccc(-c4ccc5c(C=O)cn(C)c5n4)c3Cl)c2Cl)ccc1CN(C[C@@H]1CCC(=O)N1)C(=O)OC(C)(C)C]

To a solution of 6-chloro-1-methyl-pyrrolo[2,3-b]pyridine-3-carbaldehyde (40 mg, 0.21 mmol), tert-butyl N-[[6-[2-chloro-3-[2-chloro-3-(4,4,5,5-tetramethyl-1,3,2-dioxaborolan-2-yl)phenyl]phenyl]-2-methoxy-3-pyridyl]methyl]-N-[[(2S)-5-oxopyrrolidin-2-yl]methyl]carbamate (140 mg, 0.21 mmol) and potassium carbonate (85 mg, 0.62 mmol) in degassed dioxane/water (6:1) was added Pd(PPh₃)₄ (24 mg, 0.02 mmol) and the mixture was stirred at 90° C. for 2 hours under N₂(g). Water was added and the mixture was extracted into EtOAc three times. The combined organic layers were dried over anhydrous sodium sulfate, concentrated, and purified on silica gel column using MeOH in DCM (0 to 5% gradient) to provide tert-butyl N-[[6-[2-chloro-3-[2-chloro-3-(3-formyl-1-methyl-pyrrolo[2,3-b]pyridin-6-yl)phenyl]phenyl]-2-methoxy-3-pyridyl]methyl]-N-[[(2S)-5-oxopyrrolidin-2-yl]methyl]carbamate (108 mg, 73.5%). MS: m/z found 714.2 [M+H]⁺.

### tert-Butyl N-[[6-[2-chloro-3-[2-chloro-3-[3-[[[(2S)-2-hydroxypropyl]amino]methyl]-1-methyl-pyrrolo[2,3-b]pyridin-6-yl]phenyl]phenyl]-2-methoxy-3-pyridyl]methyl]-N-[[(2S)-5-oxopyrrolidin-2-yl]methyl]carbamate

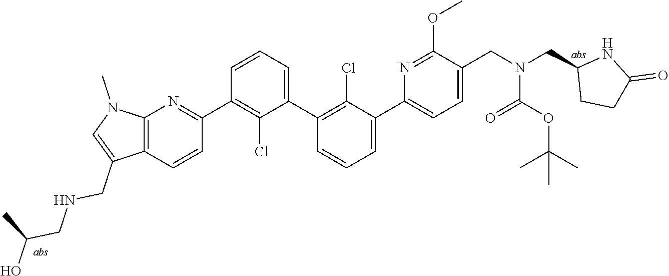 [SMILES: COc1nc(-c2cccc(-c3cccc(-c4ccc5c(CNC[C@H](C)O)cn(C)c5n4)c3Cl)c2Cl)ccc1CN(C[C@@H]1CCC(=O)N1)C(=O)OC(C)(C)C]

A mixture of tert-butyl N-[[6-[2-chloro-3-[2-chloro-3-(3-formyl-1-methyl-pyrrolo[2,3-b]pyridin-6-yl)phenyl]phenyl]-2-methoxy-3-pyridyl]methyl]-N-[[(2S)-5-oxopyrrolidin-2-yl]methyl]carbamate (20 mg, 0.03 mmol), (2S)-1-aminopropan-2-ol (4.2 mg, 0.06 mmol) and acetic acid (1.6 uL, 1.7 mg, 0.03 mmol) in MeOH/THF (1:1) was stirred for 1.5 hour at rt. NaBH₃CN (3.5 mg, 0.06 mmol) was added, the mixture was stirred for 30 min, quenched with water and directly purified by reversed phase chromatography to give tert-butyl N-[[6-[2-chloro-3-[2-chloro-3-[3-[[[(2S)-2-hydroxypropyl]amino]methyl]-1-methyl-pyrrolo[2,3-b]pyridin-6-yl]phenyl]phenyl]-2-methoxy-3-pyridyl]methyl]-N-[[(2S)-5-oxopyrrolidin-2-yl]methyl]carbamate (15 mg, 69.3%). MS: m/z found 773.3 [M+H]⁺.

### (5S)-5-[[[6-[2-Chloro-3-[2-chloro-3-[3-[[[(2S)-2-hydroxypropyl]amino]methyl]-1-methyl-pyrrolo[2,3-b]pyridin-6-yl]phenyl]phenyl]-2-methoxy-3-pyridyl]methylamino]methyl]pyrrolidin-2-one

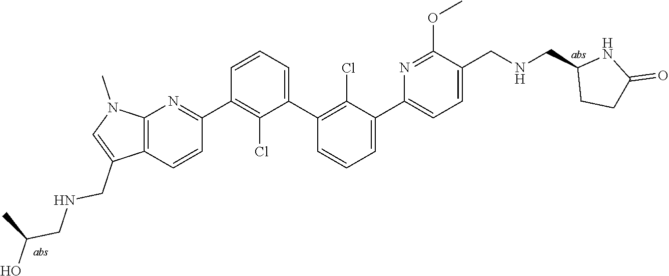 [SMILES: COc1nc(-c2cccc(-c3cccc(-c4ccc5c(CNC[C@H](C)O)cn(C)c5n4)c3Cl)c2Cl)ccc1CNC[C@@H]1CCC(=O)N1]

A solution of tert-butyl N-[[6-[2-chloro-3-[2-chloro-3-[3-[[[(2S)-2-hydroxypropyl]amino]methyl]-1-methyl-pyrrolo[2,3-b]pyridin-6-yl]phenyl]phenyl]-2-methoxy-3-pyridyl]methyl]-N-[[(2S)-5-oxopyrrolidin-2-yl]methyl]carbamate (15 mg, 0.02 mmol) in DCM/TFA (2:1) was stirred for 30 minutes, concentrated and purified by reversed phase chromatography to give (5S)-5-[[[6-[2-chloro-3-[2-chloro-3-[3-[[[(2S)-2-hydroxypropyl]amino]methyl]-1-methyl-pyrrolo[2,3-b]pyridin-6-yl]phenyl]phenyl]-2-methoxy-3-pyridyl]methylamino]methyl]pyrrolidin-2-one (12.7 mg, 97.2%). MS: m/z found 673.2 [M+H]⁺. ¹H NMR (400 MHz, Methanol-d₄): δ 8.51 (s, 1H), 8.25 (d, J=8.2 Hz, 1H), 7.75 (d, J=7.5 Hz, 1H), 7.67 (s, 1H), 7.64 (dt, J=7.6, 1.8 Hz, 2H), 7.56-7.46 (m, 3H), 7.43 (dd, J=7.6, 1.7 Hz, 1H), 7.39 (dd, J=7.6, 1.8 Hz, 1H), 7.26 (d, J=7.4 Hz, 1H), 4.48-4.42 (m, 2H), 4.10-4.03 (m, 1H), 4.03 (s, 3H), 3.94 (s, 3H), 3.91-3.88 (m, 2H), 3.88-3.83 (m, 1H), 3.10 (dd, J=12.6, 3.0 Hz, 1H), 2.88 (dd, J=12.5, 9.8 Hz, 1H), 2.82-2.70 (m, 2H), 2.38-2.24 (m, 3H), 1.87-1.76 (m, 1H), 1.22 (d, J=6.3 Hz, 3H).

### Example 412: (5S)-5-[[[6-[2-Chloro-3-[2-chloro-3-[3-[[2-hydroxyethyl(methyl)amino]methyl]-1-methyl-pyrrolo[2,3-b]pyridin-6-yl]phenyl]phenyl]-2-methoxy-3-pyridyl]methylamino]methyl]pyrrolidin-2-one

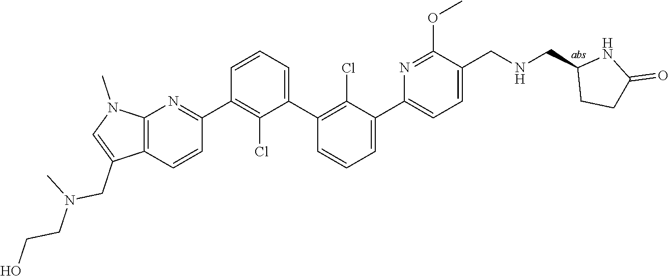 [SMILES: COc1nc(-c2cccc(-c3cccc(-c4ccc5c(CN(C)CCO)cn(C)c5n4)c3Cl)c2Cl)ccc1CNC[C@@H]1CCC(=O)N1]

### tert-Butyl N-116-[2-chloro-3-[2-chloro-3-[3-[[2-hydroxyethyl(methyl)amino]methyl]-1-methyl-pyrrolo[2,3-b]pyridin-6-yl]phenyl]phenyl]-2-methoxy-3-pyridyl]methyl]-N-[[(2S)-5-oxopyrrolidin-2-yl]methyl]carbamate

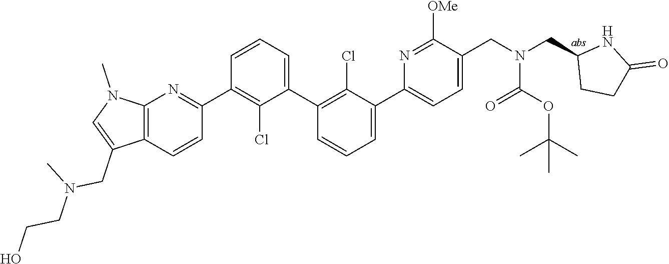 [SMILES: COc1nc(-c2cccc(-c3cccc(-c4ccc5c(CN(C)CCO)cn(C)c5n4)c3Cl)c2Cl)ccc1CN(C[C@@H]1CCC(=O)N1)C(=O)OC(C)(C)C]

A mixture of tert-butyl N-[[6-[2-chloro-3-[2-chloro-3-(3-formyl-1-methyl-pyrrolo[2,3-b]pyridin-6-yl)phenyl]phenyl]-2-methoxy-3-pyridyl]methyl]-N-[[(2S)-5-oxopyrrolidin-2-yl]methyl]carbamate (20 mg, 0.03 mmol), 2-(methylamino)ethanol (4.2 mg, 0.06 mmol) and acetic acid (1.6 uL, 1.7 mg, 0.03 mmol) in MeOH/THF (1:1) was stirred for 1.5 hour at rt. NaBH₃CN (3.52 mg, 0.06 mmol) was added, the mixture was stirred for 30 min, quenched with water and directly purified by reversed phase chromatography to give tert-butyl N-[[6-[2-chloro-3-[2-chloro-3-[3-[[2-hydroxyethyl(methyl)amino]methyl]-1-methyl-pyrrolo[2,3-b]pyridin-6-yl]phenyl]phenyl]-2-methoxy-3-pyridyl]methyl]-N-[[(2S)-5-oxopyrrolidin-2-yl]methyl]carbamate (12 mg, 55.4%). MS: m/z found 773.3 [M+H]⁺.

### 5S)-5-[[[6-[2-Chloro-3-[2-chloro-3-[3-[[2-hydroxyethyl(methyl)amino]methyl]-1-methyl-pyrrolo[2,3-b]pyridin-6-yl]phenyl]phenyl]-2-methoxy-3-pyridyl]methylamino]methyl]pyrrolidin-2-one

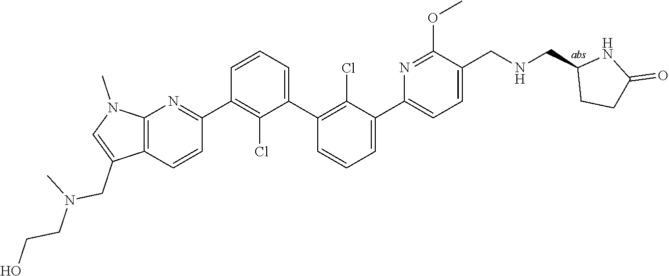 [SMILES: COc1nc(-c2cccc(-c3cccc(-c4ccc5c(CN(C)CCO)cn(C)c5n4)c3Cl)c2Cl)ccc1CNC[C@@H]1CCC(=O)N1]

A solution of tert-butyl N-[[6-[2-chloro-3-[2-chloro-3-[3-[[2-hydroxyethyl(methyl)amino]methyl]-1-methyl-pyrrolo[2,3-b]pyridin-6-yl]phenyl]phenyl]-2-methoxy-3-pyridyl]methyl]-N-[[(2S)-5-oxopyrrolidin-2-yl]methyl]carbamate (10 mg, 0.01 mmol) in DCM/TFA (2:1) was stirred for 30 minutes, concentrated and purified by reversed phase chromatography to give (5S)-5-[[[6-[2-chloro-3-[2-chloro-3-[3-[[2-hydroxyethyl(methyl)amino]methyl]-1-methyl-pyrrolo[2,3-b]pyridin-6-yl]phenyl]phenyl]-2-methoxy-3-pyridyl]methylamino]methyl]pyrrolidin-2-one (8.3 mg, 95.3%). MS: m/z found 673.2 [M+H]⁺. ¹H NMR (400 MHz, Methanol-d₄): δ 8.51 (s, 1H), 8.25 (d, J=8.2 Hz, 1H), 7.75 (d, J=7.5 Hz, 1H), 7.70 (s, 1H), 7.64 (dt, J=7.6, 1.6 Hz, 2H), 7.57-7.47 (m, 3H), 7.43 (dd, J=7.6, 1.8 Hz, 1H), 7.39 (dd, J=7.6, 1.8 Hz, 1H), 7.26 (d, J=7.4 Hz, 1H), 4.48 (s, 2H), 4.03 (s, 3H), 3.95 (s, 3H), 3.92-3.81 (m, 5H), 3.20 (s, 2H), 2.81 (s, 3H), 2.78-2.66 (m, 2H), 2.41-2.20 (m, 3H), 1.91-1.73 (m, 1H).

### Example 413: (5S)-5-[[[6-[2-Chloro-3-[2-chloro-3-[1-methyl-3-[[[(2S)-oxetan-2-yl]methylamino]methyl]pyrrolo[2,3-b]pyridin-6-yl]phenyl]phenyl]-2-methoxy-3-pyridyl]methylamino]methyl]pyrrolidin-2-one

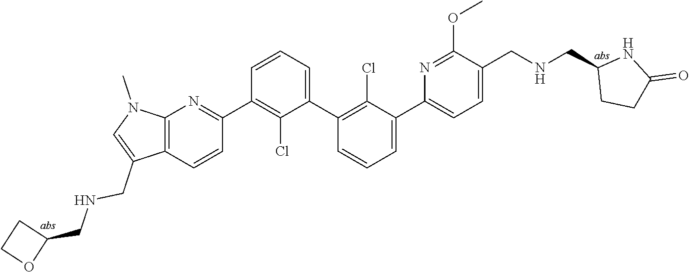 [SMILES: COc1nc(-c2cccc(-c3cccc(-c4ccc5c(CNC[C@@H]6CCO6)cn(C)c5n4)c3Cl)c2Cl)ccc1CNC[C@@H]1CCC(=O)N1]

### tert-Butyl N-[[6-[2-chloro-3-[2-chloro-3-[1-methyl-3-[[[(2S)-oxetan-2-yl]methylamino]methyl]pyrrolo[2,3-b]pyridin-6-yl]phenyl]phenyl]-2-methoxy-3-pyridyl]methyl]-N-[[(2S)-5-oxopyrrolidin-2-yl]methyl]carbamate

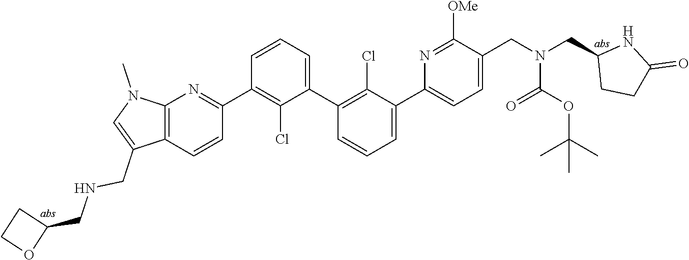 [SMILES: COc1nc(-c2cccc(-c3cccc(-c4ccc5c(CNC[C@@H]6CCO6)cn(C)c5n4)c3Cl)c2Cl)ccc1CN(C[C@@H]1CCC(=O)N1)C(=O)OC(C)(C)C]

A mixture of tert-butyl N-[[6-[2-chloro-3-[2-chloro-3-(3-formyl-1-methyl-pyrrolo[2,3-b]pyridin-6-yl)phenyl]phenyl]-2-methoxy-3-pyridyl]methyl]-N-[[(2S)-5-oxopyrrolidin-2-yl]methyl]carbamate (20 mg, 0.03 mmol), [(2S)-oxetan-2-yl]methanamine (5 mg, 0.06 mmol) and acetic acid (1.6 uL, 1.7 mg, 0.03 mmol) in MeOH/THF (1:1) was stirred for 1.5 hour at rt. NaBH₃CN (3.5 mg, 0.06 mmol) was added, the mixture was stirred for 30 min, quenched with water and directly purified by reversed phase chromatography to give tert-butyl N-[[6-[2-chloro-3-[2-chloro-3-[1-methyl-3-[[[(2S)-oxetan-2-yl]methylamino]methyl]pyrrolo[2,3-b]pyridin-6-yl]phenyl]phenyl]-2-methoxy-3-pyridyl]methyl]-N-[[(2S)-5-oxopyrrolidin-2-yl]methyl]carbamate (16 mg, 72.8%). MS: m/z found 785.3 [M+H]⁺.

### (5S)-5-[[[6-[2-Chloro-3-[2-chloro-3-[1-methyl-3-[[[(2S)-oxetan-2-yl]methylamino]methyl]pyrrolo[2,3-b]pyridin-6-yl]phenyl]phenyl]-2-methoxy-3-pyridyl]methylamino]methyl]pyrrolidin-2-one

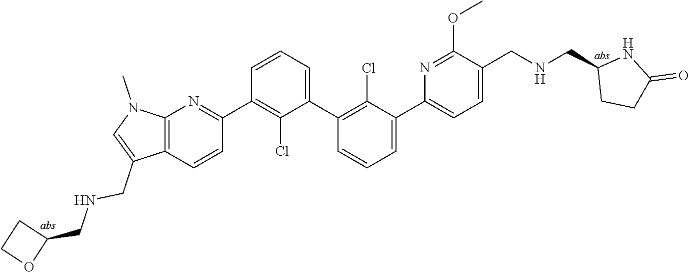 [SMILES: COc1nc(-c2cccc(-c3cccc(-c4ccc5c(CNC[C@@H]6CCO6)cn(C)c5n4)c3Cl)c2Cl)ccc1CNC[C@@H]1CCC(=O)N1]

A solution of tert-butyl N-[[6-[2-chloro-3-[2-chloro-3-[1-methyl-3-[[[(2S)-oxetan-2-yl]methylamino]methyl]pyrrolo[2,3-b]pyridin-6-yl]phenyl]phenyl]-2-methoxy-3-pyridyl]methyl]-N-[[(2S)-5-oxopyrrolidin-2-yl]methyl]carbamate (10 mg, 0.01 mmol) in DCM/TFA (2:1) was stirred for 30 minutes, concentrated and purified by reversed phase chromatography to give (5S)-5-[[[6-[2-chloro-3-[2-chloro-3-[1-methyl-3-[[[(2S)-oxetan-2-yl]methylamino]methyl]pyrrolo[2,3-b]pyridin-6-yl]phenyl]phenyl]-2-methoxy-3-pyridyl]methylamino]methyl]pyrrolidin-2-one (8.3 mg, 95.1%). MS: m/z found 685.2 [M+H]⁺. ¹H NMR (400 MHz, Methanol-d₄): δ 8.53 (s, 1H), 8.23 (d, J=8.2 Hz, 1H), 7.74 (d, J=7.5 Hz, 1H), 7.64 (dt, J=7.6, 1.9 Hz, 2H), 7.61 (s, 1H), 7.55-7.51 (m, 1H), 7.49 (t, J=6.9 Hz, 1H), 7.46-7.41 (m, 2H), 7.39 (dd, J=7.5, 1.8 Hz, 1H), 7.25 (d, J=7.4 Hz, 1H), 5.06 (qd, J=7.9, 3.3 Hz, 1H), 4.72 (ddd, J=8.6, 7.4, 5.9 Hz, 1H), 4.61 (dt, J=9.2, 6.1 Hz, 1H), 4.40-4.30 (m, 2H), 4.02 (s, 3H), 3.93 (s, 3H), 3.90-3.80 (m, 3H), 3.35 (dd, J=13.0, 8.3 Hz, 1H), 3.15 (dd, J=13.0, 3.4 Hz, 1H), 2.85-2.66 (m, 3H), 2.56-2.45 (m, 1H), 2.37-2.23 (m, 3H), 1.87-1.76 (m, 1H).

### Example 414: 1-[2-[[6-[2-Chloro-3-[2-chloro-3-[6-methoxy-5-[[[(2S)-5-oxopyrrolidin-2-yl]methylamino]methyl]-2-pyridyl]phenyl]phenyl]-1-methyl-pyrrolo[2,3-b]pyridin-3-yl]methylamino]ethyl]pyrrolidin-2-one

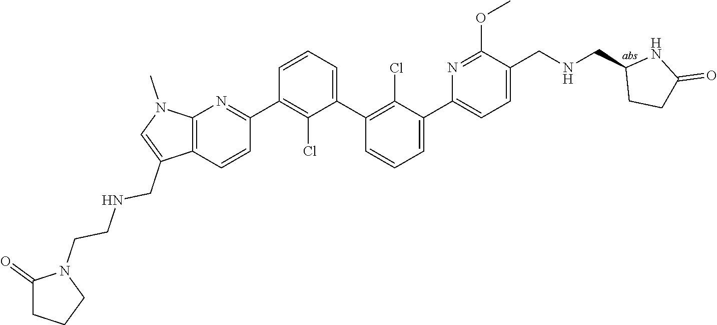 [SMILES: COc1nc(-c2cccc(-c3cccc(-c4ccc5c(CNCCN6CCCC6=O)cn(C)c5n4)c3Cl)c2Cl)ccc1CNC[C@@H]1CCC(=O)N1]

### tert-Butyl N-[[6-[2-chloro-3-[2-chloro-3-[1-methyl-3-[[2-(2-oxopyrrolidin-1-yl)ethylamino]methyl]pyrrolo[2,3-b]pyridin-6-yl]phenyl]phenyl]-2-methoxy-3-pyridyl]methyl]-N-[[(2S)-5-oxopyrrolidin-2-yl]methyl]carbamate

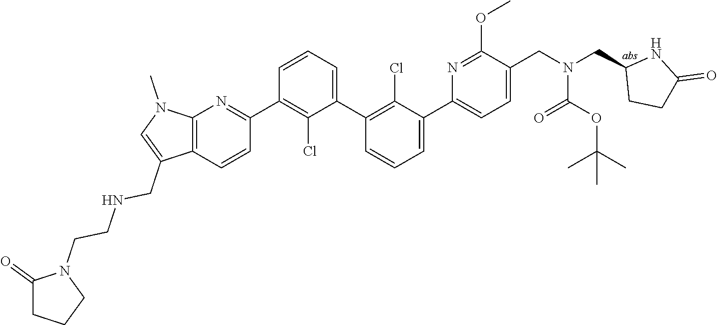 [SMILES: COc1nc(-c2cccc(-c3cccc(-c4ccc5c(CNCCN6CCCC6=O)cn(C)c5n4)c3Cl)c2Cl)ccc1CN(C[C@@H]1CCC(=O)N1)C(=O)OC(C)(C)C]

A mixture of tert-butyl N-[[6-[2-chloro-3-[2-chloro-3-(3-formyl-1-methyl-pyrrolo[2,3-b]pyridin-6-yl)phenyl]phenyl]-2-methoxy-3-pyridyl]methyl]-N-[[(2S)-5-oxopyrrolidin-2-yl]methyl]carbamate (20 mg, 0.03 mmol), 1-(2-aminoethyl)pyrrolidin-2-one (7 mg, 0.06 mmol) and acetic acid (1.6 uL, 1.7 mg, 0.03 mmol) in MeOH/THF (1:1) was stirred for 1.5 hour at rt. NaBH₃CN (3.2 mg, 0.06 mmol) was added, the mixture was stirred for 30 min, quenched with water and directly purified by reversed phase chromatography to give tert-butyl N-[[6-[2-chloro-3-[2-chloro-3-[1-methyl-3-[[2-(2-oxopyrrolidin-1-yl)ethylamino]methyl]pyrrolo[2,3-b]pyridin-6-yl]phenyl]phenyl]-2-methoxy-3-pyridyl]methyl]-N-[[(2S)-5-oxopyrrolidin-2-yl]methyl]carbamate (17 mg, 73.5%). MS: m/z found 826.3 [M+H]⁺.

### 1-[2-[[6-[2-Chloro-3-[2-chloro-3-[6-methoxy-5-[[[(2S)-5-oxopyrrolidin-2-yl]methylamino]methyl]-2-pyridyl]phenyl]phenyl]-1-methyl-pyrrolo[2,3-b]pyridin-3-yl]methylamino]ethyl]pyrrolidin-2-one

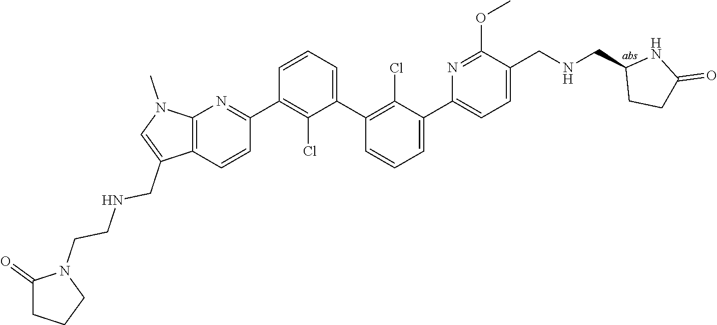 [SMILES: COc1nc(-c2cccc(-c3cccc(-c4ccc5c(CNCCN6CCCC6=O)cn(C)c5n4)c3Cl)c2Cl)ccc1CNC[C@@H]1CCC(=O)N1]

A solution of tert-butyl N-[[6-[2-chloro-3-[2-chloro-3-[1-methyl-3-[[2-(2-oxopyrrolidin-1-yl)ethylamino]methyl]pyrrolo[2,3-b]pyridin-6-yl]phenyl]phenyl]-2-methoxy-3-pyridyl]methyl]-N-[[(2S)-5-oxopyrrolidin-2-yl]methyl]carbamate (10 mg, 0.01 mmol) in DCM/TFA (2:1) was stirred for 30 minutes, concentrated and purified by reversed phase chromatography to give 1-[2-[[6-[2-chloro-3-[2-chloro-3-[6-methoxy-5-[[[(2S)-5-oxopyrrolidin-2-yl]methylamino]methyl]-2-pyridyl]phenyl]phenyl]-1-methyl-pyrrolo[2,3-b]pyridin-3-yl]methylamino]ethyl]pyrrolidin-2-one (8.2 mg, 93.3%). MS: m/z found 726.3 [M+H]⁺. ¹H NMR (400 MHz, Methanol-d₄): δ 8.51 (s, 1H), 8.26 (d, J=8.1 Hz, 1H), 7.76 (d, J=7.5 Hz, 1H), 7.66-7.61 (m, 3H), 7.56-7.52 (m, 1H), 7.52-7.48 (m, 1H), 7.46 (d, J=8.2 Hz, 1H), 7.43 (dd, J=7.6, 1.7 Hz, 1H), 7.40 (dd, J=7.6, 1.7 Hz, 1H), 7.27 (d, J=7.4 Hz, 1H), 4.43 (s, 2H), 4.03 (s, 3H), 3.93 (s, 3H), 3.92-3.89 (m, 2H), 3.88-3.84 (m, 1H), 3.62 (t, J=5.9 Hz, 2H), 3.48 (t, J=7.1 Hz, 2H), 3.23 (t, J=5.9 Hz, 2H), 2.83-2.71 (m, 2H), 2.42-2.24 (m, 5H), 2.06 (p, J=7.7 Hz, 2H), 1.88-1.78 (m, 1H).

### Example 415: (5S)-5-[[[6-[3-[3-[3-[[(1-Acetyl-4-piperidyl)amino]methyl]-1-methyl-pyrrolo[2,3-b]pyridin-6-yl]-2-chloro-phenyl]-2-chloro-phenyl]-2-methoxy-3-pyridyl]methylamino]methyl]pyrrolidin-2-one

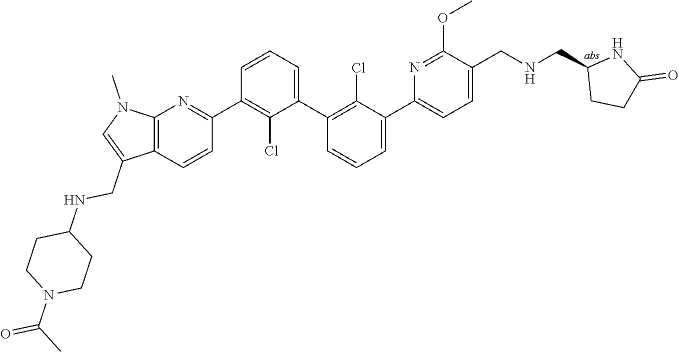 [SMILES: COc1nc(-c2cccc(-c3cccc(-c4ccc5c(CNC6CCN(C(C)=O)CC6)cn(C)c5n4)c3Cl)c2Cl)ccc1CNC[C@@H]1CCC(=O)N1]

### tert-Butyl N-[[6-[3-[3-[3-[[(1-Acetyl-4-piperidyl)amino]methyl]-1-methyl-pyrrolo[2,3-b]pyridin-6-yl]-2-chloro-phenyl]-2-chloro-phenyl]-2-methoxy-3-pyridyl]methyl]-N-[[(2S)-5-oxopyrrolidin-2-yl]methyl]carbamate

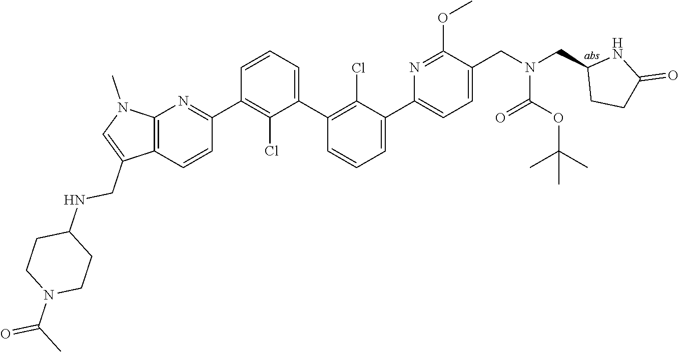 [SMILES: COc1nc(-c2cccc(-c3cccc(-c4ccc5c(CNC6CCN(C(C)=O)CC6)cn(C)c5n4)c3Cl)c2Cl)ccc1CN(C[C@@H]1CCC(=O)N1)C(=O)OC(C)(C)C]

A mixture of tert-butyl N-[[6-[2-chloro-3-[2-chloro-3-(3-formyl-1-methyl-pyrrolo[2,3-b]pyridin-6-yl)phenyl]phenyl]-2-methoxy-3-pyridyl]methyl]-N-[[(2S)-5-oxopyrrolidin-2-yl]methyl]carbamate (20 mg, 0.03 mmol), 1-(4-amino-1-piperidyl)ethanone (8 mg, 0.06 mmol) and acetic acid (1.6 uL, 1.7 mg, 0.03 mmol) in MeOH/THF (1:1) was stirred for 1.5 hour at rt. NaBH₃CN (3.5 mg, 0.06 mmol) was added, the mixture was stirred for 30 min, quenched with water and directly purified by reversed phase chromatography to give tert-butyl N-[[6-[3-[3-[3-[[(1-acetyl-4-piperidyl)amino]methyl]-1-methyl-pyrrolo[2,3-b]pyridin-6-yl]-2-chloro-phenyl]-2-chloro-phenyl]-2-methoxy-3-pyridyl]methyl]-N-[[(2S)-5-oxopyrrolidin-2-yl]methyl]carbamate (17 mg, 72.2%). MS: m/z found 840.3 [M+H]⁺.

### (5S)-5-[[[6-[3-[3-[3-[[(1-Acetyl-4-piperidyl)amino]methyl]-1-methyl-pyrrolo[2,3-b]pyridin-6-yl]-2-chloro-phenyl]-2-chloro-phenyl]-2-methoxy-3-pyridyl]methylamino]methyl]pyrrolidin-2-one

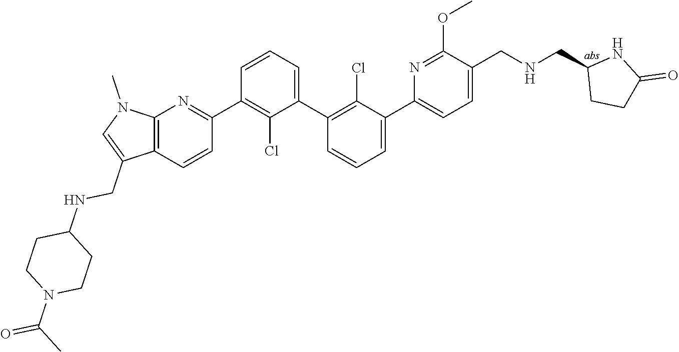 [SMILES: COc1nc(-c2cccc(-c3cccc(-c4ccc5c(CNC6CCN(C(C)=O)CC6)cn(C)c5n4)c3Cl)c2Cl)ccc1CNC[C@@H]1CCC(=O)N1]

A solution of tert-butyl N-[[6-[3-[3-[3-[[(1-acetyl-4-piperidyl)amino]methyl]-1-methyl-pyrrolo[2,3-b]pyridin-6-yl]-2-chloro-phenyl]-2-chloro-phenyl]-2-methoxy-3-pyridyl]methyl]-N-[[(2S)-5-oxopyrrolidin-2-yl]methyl]carbamate (10 mg, 0.01 mmol) in DCM/TFA (2:1) was stirred for 30 minutes, concentrated and purified by reversed phase chromatography to give (5S)-5-[[[6-[3-[3-[3-[[(1-acetyl-4-piperidyl)amino]methyl]-1-methyl-pyrrolo[2,3-b]pyridin-6-yl]-2-chloro-phenyl]-2-chloro-phenyl]-2-methoxy-3-pyridyl]methylamino]methyl]pyrrolidin-2-one (8.2 mg, 93.1%). MS: m/z found 740.3 [M+H]⁺. ¹H NMR (400 MHz, Methanol-d₄): δ 8.51 (s, 1H), 8.23 (d, J=8.2 Hz, 1H), 7.75 (d, J=7.5 Hz, 1H), 7.68-7.60 (m, 3H), 7.56-7.45 (m, 3H), 7.43 (dd, J=7.6, 1.7 Hz, 1H), 7.39 (dd, J=7.6, 1.8 Hz, 1H), 7.26 (d, J=7.4 Hz, 1H), 4.65 (d, J=13.7 Hz, 1H), 4.43 (s, 2H), 4.10-4.03 (m, 1H), 4.03 (s, 3H), 3.94 (s, 3H), 3.90-3.86 (m, 2H), 3.86-3.82 (m, 1H), 3.42-3.34 (m, 1H), 3.25-3.15 (m, 1H), 2.80-2.65 (m, 3H), 2.38-2.31 (m, 2H), 2.30-2.19 (m, 3H), 2.13 (s, 3H), 1.88-1.75 (m, 1H), 1.66-1.54 (m, 1H), 1.55-1.44 (m, 1H).

### Example 416: 8-[2-Chloro-3-[2-chloro-3-[1-methyl-3-[[[(2S)-5-oxopyrrolidin-2-yl]methylamino]methyl]pyrrolo[2,3-b]pyridin-6-yl]phenyl]phenyl]-3-[[[(2S)-5-oxopyrrolidin-2-yl]methylamino]methyl]pyrido[1,2-a]pyrimidin-4-one

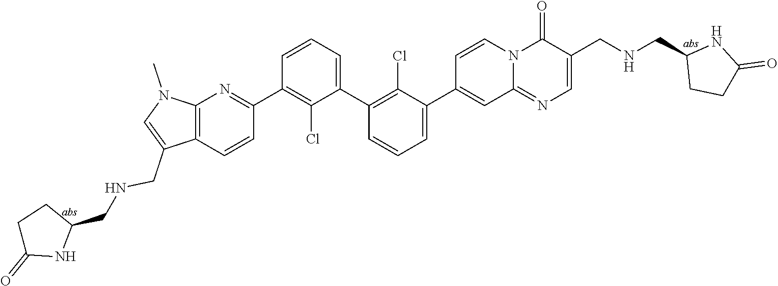 [SMILES: Cn1cc(CNC[C@@H]2CCC(=O)N2)c2ccc(-c3cccc(-c4cccc(-c5ccn6c(=O)c(CNC[C@@H]7CCC(=O)N7)cnc6c5)c4Cl)c3Cl)nc21]

### 8-[2-Chloro-3-[2-chloro-3-(3-formyl-1-methyl-pyrrolo[2,3-b]pyridin-6-yl)phenyl]phenyl]-4-oxo-pyrido[1,2-a]pyrimidine-3-carbaldehyde

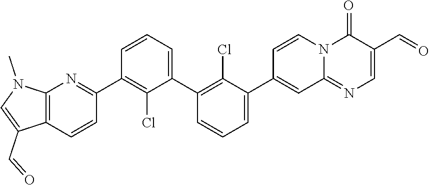 [SMILES: Cn1cc(C=O)c2ccc(-c3cccc(-c4cccc(-c5ccn6c(=O)c(C=O)cnc6c5)c4Cl)c3Cl)nc21]

To a solution of 6-chloro-1-methyl-pyrrolo[2,3-b]pyridine-3-carbaldehyde (20 mg, 0.10 mmol), 8-[2-chloro-3-[2-chloro-3-(4,4,5,5-tetramethyl-1,3,2-dioxaborolan-2-yl)phenyl]phenyl]-4-oxo-pyrido[1,2-a]pyrimidine-3-carbaldehyde (54 mg, 0.10 mmol and potassium carbonate (43 mg, 0.31 mmol) in degassed dioxane/water (6:1) was added Pd(PPh₃)₄ (12 mg, 0.01 mmol) and the mixture was stirred at 90° C. for 2 hours under N₂(g). Water was added and the mixture was extracted into EtOAc three times. The combined organic layers were dried over anhydrous sodium sulfate, concentrated, and purified on silica gel column using MeOH in DCM (0 to 5% gradient) to provide 8-[2-chloro-3-[2-chloro-3-(3-formyl-1-methyl-pyrrolo[2,3-b]pyridin-6-yl)phenyl]phenyl]-4-oxo-pyrido[1,2-a]pyrimidine-3-carbaldehyde (35 mg, 61.5%). MS: m/z found 553.1 [M+H]⁺.

### 8-[2-Chloro-3-[2-chloro-3-[1-methyl-3-[[[(2S)-5-oxopyrrolidin-2-yl]methylamino]methyl]pyrrolo[2,3-b]pyridin-6-yl]phenyl]phenyl]-3-[[[(2S)-5-oxopyrrolidin-2-yl]methylamino]methyl]pyrido[1,2-a]pyrimidin-4-one

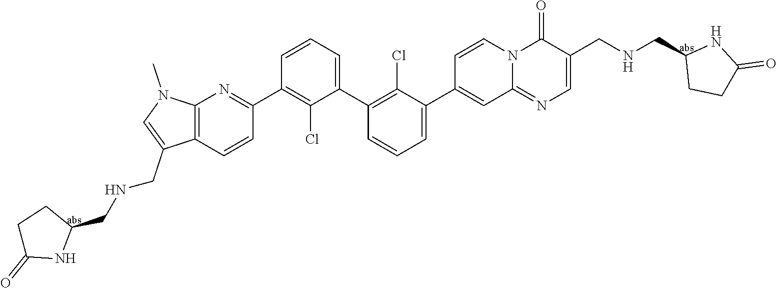 [SMILES: Cn1cc(CNC[C@@H]2CCC(=O)N2)c2ccc(-c3cccc(-c4cccc(-c5ccn6c(=O)c(CNC[C@@H]7CCC(=O)N7)cnc6c5)c4Cl)c3Cl)nc21]

A mixture of 8-[2-chloro-3-[2-chloro-3-(3-formyl-1-methyl-pyrrolo[2,3-b]pyridin-6-yl)phenyl]phenyl]-4-oxo-pyrido[1,2-a]pyrimidine-3-carbaldehyde (25 mg, 0.05 mmol), (5S)-5-(aminomethyl)pyrrolidin-2-one (21 mg, 0.18 mmol) and acetic acid (5 uL, 5.4 mg, 0.09 mmol) in MeOH/THF (1:1) was stirred for 1.5 hour at rt. NaBH₃CN (11 mg, 0.18 mmol) was added, the mixture was stirred for additional 20 min, quenched with water and directly purified by reversed phase chromatography to give 8-[2-chloro-3-[2-chloro-3-[1-methyl-3-[[[(2S)-5-oxopyrrolidin-2-yl]methylamino]methyl]pyrrolo[2,3-b]pyridin-6-yl]phenyl]phenyl]-3-[[[(2S)-5-oxopyrrolidin-2-yl]methylamino]methyl]pyrido[1,2-a]pyrimidin-4-one (27 mg, 79.7%). MS: m/z found 749.3 [M+H]⁺. ¹H NMR (400 MHz, Methanol-d₄): δ 9.16 (d, J=7.4 Hz, 1H), 8.45 (s, 1H), 8.24 (d, J=8.2 Hz, 1H), 7.80 (d, J=1.8 Hz, 1H), 7.64 (ddd, J=16.0, 7.6, 1.4 Hz, 4H), 7.60-7.52 (m, 3H), 7.45 (dd, J=7.8, 1.5 Hz, 2H), 4.40-4.29 (m, 2H), 4.06-3.99 (m, 2H), 3.98-3.89 (m, 2H), 3.93 (s, 3H) 3.06 (h, J=7.1 Hz, 2H), 2.89 (qd, J=12.3, 6.1 Hz, 2H), 2.43-2.25 (m, 6H), 1.92-1.80 (m, 2H).

### Example 417: (5S)-5-[[[6-[2-Chloro-3-[2-chloro-3-[1-methyl-3-[[[(2S)-5-oxopyrrolidin-2-yl]methylamino]methyl]pyrrolo[2,3-b]pyridin-6-yl]phenyl]phenyl]-1-methyl-pyrrolo[2,3-b]pyridin-3-yl]methylamino]methyl]pyrrolidin-2-one

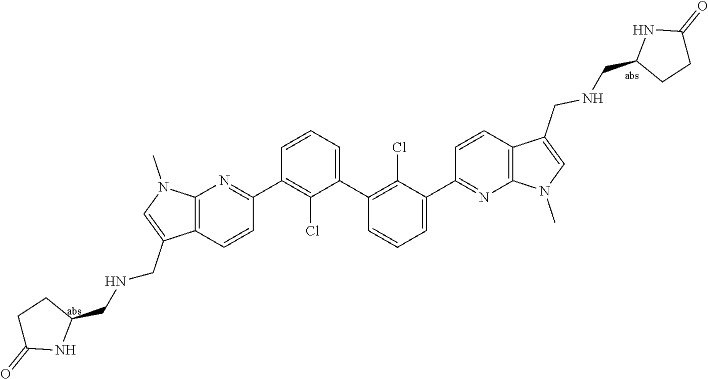 [SMILES: Cn1cc(CNC[C@@H]2CCC(=O)N2)c2ccc(-c3cccc(-c4cccc(-c5ccc6c(CNC[C@@H]7CCC(=O)N7)cn(C)c6n5)c4Cl)c3Cl)nc21]

### 6-[2-Chloro-3-[2-chloro-3-(3-formyl-1-methyl-pyrrolo[2,3-b]pyridin-6-yl)phenyl]phenyl]-1-methyl-pyrrolo[2,3-b]pyridine-3-carbaldehyde

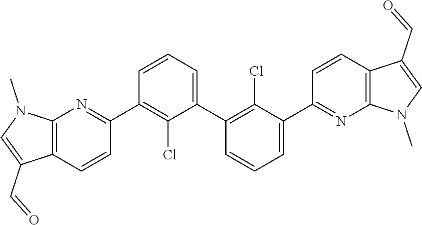 [SMILES: Cn1cc(C=O)c2ccc(-c3cccc(-c4cccc(-c5ccc6c(C=O)cn(C)c6n5)c4Cl)c3Cl)nc21]

To a solution of 6-chloro-1-methyl-pyrrolo[2,3-b]pyridine-3-carbaldehyde (55 mg, 0.28 mmol), 2-[2-chloro-3-[2-chloro-3-(4,4,5,5-tetramethyl-1,3,2-dioxaborolan-2-yl)phenyl]phenyl]-4,4,5,5-tetramethyl-1,3,2-dioxaborolane (45 mg, 0.09 mmol) and potassium carbonate (65 mg, 0.47 mmol) in degassed dioxane/water (6:1) was added Pd(PPh₃)₄ (22 mg, 0.02 mmol) and the mixture was stirred at 90° C. for 3 hours under N₂(g). Water was added and the mixture was extracted into EtOAc three times. The combined organic layers were dried over anhydrous sodium sulfate, concentrated, and purified on silica gel column to provide 6-[2-chloro-3-[2-chloro-3-(3-formyl-1-methyl-pyrrolo[2,3-b]pyridin-6-yl)phenyl]phenyl]-1-methyl-pyrrolo[2,3-b]pyridine-3-carbaldehyde (40 mg, 78.3%). MS: m/z found 539.1 [M+H]⁺.

### (5S)-5-[[[6-[2-Chloro-3-[2-chloro-3-[1-methyl-3-[[[(2S)-5-oxopyrrolidin-2-yl]methylamino]methyl]pyrrolo[2,3-b]pyridin-6-yl]phenyl]phenyl]-1-methyl-pyrrolo[2,3-b]pyridin-3-yl]methylamino]methyl]pyrrolidin-2-one

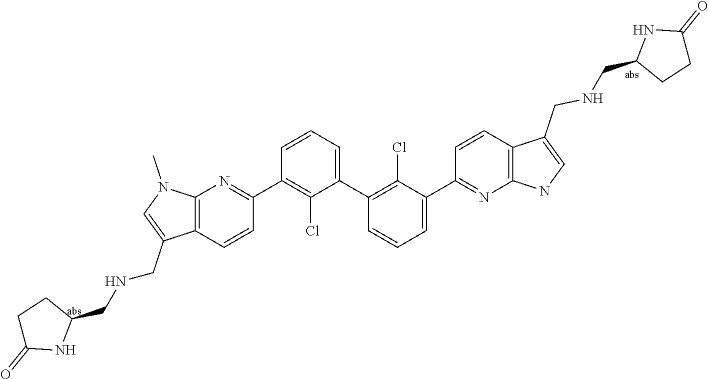 [SMILES: Cn1cc(CNC[C@@H]2CCC(=O)N2)c2ccc(-c3cccc(-c4cccc(-c5ccc6c(CNC[C@@H]7CCC(=O)N7)c[nH]c6n5)c4Cl)c3Cl)nc21]

A mixture of 6-[2-chloro-3-[2-chloro-3-(3-formyl-1-methyl-pyrrolo[2,3-b]pyridin-6-yl)phenyl]phenyl]-1-methyl-pyrrolo[2,3-b]pyridine-3-carbaldehyde (20 mg, 0.04 mmol), (5S)-5-(aminomethyl)pyrrolidin-2-one (17 mg, 0.15 mmol) and acetic acid (4. uL, 4.5 mg, 0.07 mmol) in MeOH/THF (1:1) was stirred for 1.5 hour at rt. NaBH₃CN (9 mg, 0.15 mmol) was added, the mixture was stirred for additional 20 min, quenched with water and directly purified by reversed phase chromatography to give (5S)-5-[[[6-[2-chloro-3-[2-chloro-3-[1-methyl-3-[[[(2S)-5-oxopyrrolidin-2-yl]methylamino]methyl]pyrrolo[2,3-b]pyridin-6-yl]phenyl]phenyl]-1-methyl-pyrrolo[2,3-b]pyridin-3-yl]methylamino]methyl]pyrrolidin-2-one (17 mg, 62.3%). MS: m/z found 735.3 [M+H]⁺. ¹H NMR (400 MHz, Methanol-d₄): δ 8.20 (d, J=8.1 Hz, 2H), 7.64 (dd, J=7.6, 1.8 Hz, 2H), 7.54 (t, J=7.6 Hz, 2H), 7.49 (s, 2H), 7.44 (dd, J=7.6, 1.8 Hz, 2H), 7.40 (d, J=8.1 Hz, 2H), 4.21-4.10 (m, 4H), 3.92-3.85 (m, 2H), 3.90 (s, 6H), 2.94-2.79 (m, 4H), 2.39-2.25 (m, 6H), 1.89-1.75 (m, 2H).

### Example 429: 8-[2-Chloro-3-[2-chloro-3-[3-chloro-2-[[[(2S)-5-oxopyrrolidin-2-yl]methylamino]methyl]imidazo[1,2-a]pyridin-7-yl]phenyl]phenyl]-3-[[[(2S)-5-oxopyrrolidin-2-yl]methylamino]methyl]pyrido[1,2-a]pyrimidin-4-one

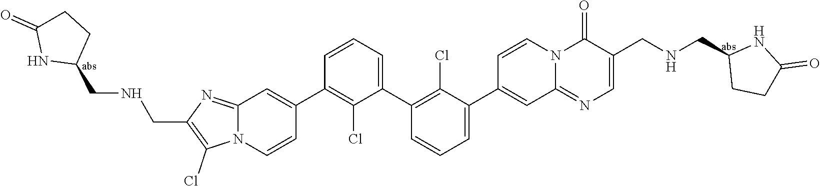 [SMILES: O=C1CC[C@@H](CNCc2nc3cc(-c4cccc(-c5cccc(-c6ccn7c(=O)c(CNC[C@@H]8CCC(=O)N8)cnc7c6)c5Cl)c4Cl)ccn3c2Cl)N1]

### 8-[2-Chloro-3-[2-chloro-3-(3-chloro-2-formyl-imidazo[1,2-a]pyridin-7-yl)phenyl]phenyl]-4-oxo-pyrido[1,2-a]pyrimidine-3-carbaldehyde

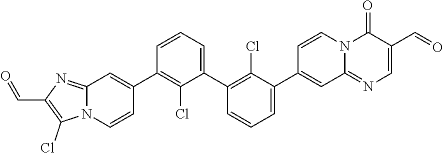 [SMILES: O=Cc1nc2cc(-c3cccc(-c4cccc(-c5ccn6c(=O)c(C=O)cnc6c5)c4Cl)c3Cl)ccn2c1Cl]

To a solution of 7-bromo-3-chloro-imidazo[1,2-a]pyridine-2-carbaldehyde (20 mg, 0.08 mmol), 8-[2-chloro-3-[2-chloro-3-(4,4,5,5-tetramethyl-1,3,2-dioxaborolan-2-yl)phenyl]phenyl]-4-oxo-pyrido[1,2-a]pyrimidine-3-carbaldehyde (40 mg, 0.08 mmol) and potassium carbonate (32 mg, 0.23 mmol) in degassed dioxane/water (6:1) was added Pd(PPh₃)₄ (9 mg, 0.01 mmol) and the mixture was stirred at 90° C. for 2 hours under N₂(g). Water was added and the mixture was extracted into EtOAc three times. The combined organic layers were dried over anhydrous sodium sulfate, concentrated, and purified on silica gel column to provide 8-[2-chloro-3-[2-chloro-3-(3-chloro-2-formyl-imidazo[1,2-a]pyridin-7-yl)phenyl]phenyl]-4-oxo-pyrido[1,2-a]pyrimidine-3-carbaldehyde (10 mg, 22.6%). MS: m/z found 573.0 [M+H]⁺.

### 8-[2-Chloro-3-[2-chloro-3-[3-chloro-2-[[[(2S)-5-oxopyrrolidin-2-yl]methylamino]methyl]imidazo[1,2-a]pyridin-7-yl]phenyl]phenyl]-3-[[[(2S)-5-oxopyrrolidin-2-yl]methylamino]methyl]pyrido[1,2-a]pyrimidin-4-one

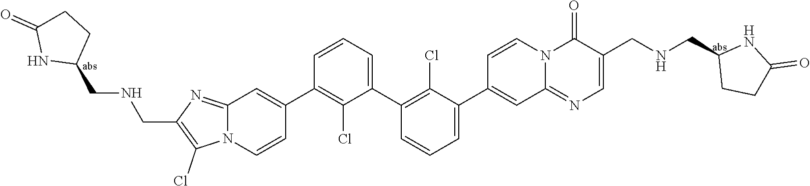 [SMILES: O=C1CC[C@@H](CNCc2nc3cc(-c4cccc(-c5cccc(-c6ccn7c(=O)c(CNC[C@@H]8CCC(=O)N8)cnc7c6)c5Cl)c4Cl)ccn3c2Cl)N1]

A mixture of 8-[2-chloro-3-[2-chloro-3-(3-chloro-2-formyl-imidazo[1,2-a]pyridin-7-yl)phenyl]phenyl]-4-oxo-pyrido[1,2-a]pyrimidine-3-carbaldehyde (8 mg, 0.01 mmol), (5S)-5-(aminomethyl)pyrrolidin-2-one (6.4 mg, 0.06 mmol) and acetic acid (1.6 uL, 1.7 mg, 0.03 mmol) in MeOH/THF (1:1) was stirred for 1.5 hour at rt. NaBH₃CN (3.5 mg, 0.06 mmol) was added, the mixture was stirred for additional 20 min and quenched with water and directly purified by reversed phase chromatography to give 8-[2-chloro-3-[2-chloro-3-[3-chloro-2-[[[(2S)-5-oxopyrrolidin-2-yl]methylamino]methyl]imidazo[1,2-a]pyridin-7-yl]phenyl]phenyl]-3-[[[(2S)-5-oxopyrrolidin-2-yl]methylamino]methyl]pyrido[1,2-a]pyrimidin-4-one (7 mg, 65.2%). MS: m/z found 769.2 [M+H]⁺. ¹H NMR (400 MHz, Methanol-d₄): δ 9.17 (d, J=7.4 Hz, 1H), 8.46 (s, 1H), 8.38 (dt, J=7.0, 1.6 Hz, 1H), 7.82-7.79 (m, 1H), 7.68-7.52 (m, 7H), 7.51-7.45 (m, 1H), 7.45-7.38 (m, 1H), 7.26 (dd, J=7.1, 1.7 Hz, 1H), 4.11-3.98 (m, 4H), 3.96-3.89 (m, 1H), 3.88-3.78 (m, 1H), 2.97-2.83 (m, 2H), 2.82-2.70 (m, 2H), 2.41-2.21 (m, 6H), 1.92-1.75 (m, 2H).

### Example 430: (5S)-5-[[[3-Chloro-7-[2-chloro-3-[2-chloro-3-[3-chloro-2-[[[(2S)-5-oxopyrrolidin-2-yl]methylamino]methyl]imidazo[1,2-a]pyridin-7-yl]phenyl]phenyl]imidazo[1,2-a]pyridin-2-yl]methylamino]methyl]pyrrolidin-2-one

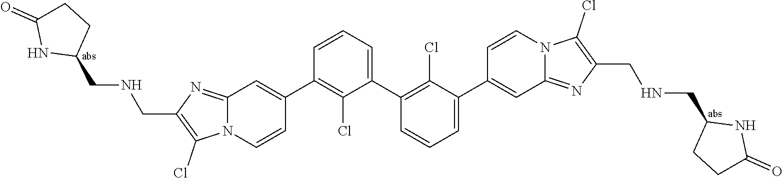 [SMILES: O=C1CC[C@@H](CNCc2nc3cc(-c4cccc(-c5cccc(-c6ccn7c(Cl)c(CNC[C@@H]8CCC(=O)N8)nc7c6)c5Cl)c4Cl)ccn3c2Cl)N1]

### 3-Chloro-7-[2-chloro-3-[2-chloro-3-(3-chloro-2-formyl-imidazo[1,2-a]pyridin-7-yl)phenyl]phenyl]imidazo[1,2-a]pyridine-2-carbaldehyde

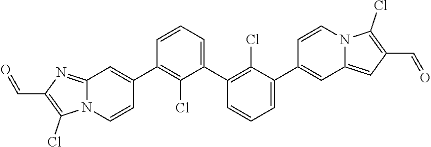 [SMILES: O=Cc1cc2cc(-c3cccc(-c4cccc(-c5ccn6c(Cl)c(C=O)nc6c5)c4Cl)c3Cl)ccn2c1Cl]

To a solution of 7-bromo-3-chloro-imidazo[1,2-a]pyridine-2-carbaldehyde (38 mg, 0.15 mmol), 2-[2-chloro-3-[2-chloro-3-(4,4,5,5-tetramethyl-1,3,2-dioxaborolan-2-yl)phenyl]phenyl]-4,4,5,5-tetramethyl-1,3,2-dioxaborolane (35 mg, 0.07 mmol) and potassium carbonate (51 mg, 0.37 mmol) in degassed dioxane/water (6:1) was added Pd(PPh₃)₄ (17 mg, 0.01 mmol) and the mixture was stirred at 90° C. for 3 hours under N₂(g). Water was added and the mixture was extracted into EtOAc three times. The combined organic layers were dried over anhydrous sodium sulfate, concentrated, and purified on silica gel column to provide 3-chloro-7-[2-chloro-3-[2-chloro-3-(3-chloro-2-formyl-imidazo[1,2-a]pyridin-7-yl)phenyl]phenyl]imidazo[1,2-a]pyridine-2-carbaldehyde (8 mg, 18.7%). MS: m/z found 579.0 [M+H]⁺.

### (5S)-5-[[[3-Chloro-7-[2-chloro-3-[2-chloro-3-[3-chloro-2-[[[(2S)-5-oxopyrrolidin-2-yl]methylamino]methyl]imidazo[1,2-a]pyridin-7-yl]phenyl]phenyl]imidazo[1,2-a]pyridin-2-yl]methylamino]methyl]pyrrolidin-2-one

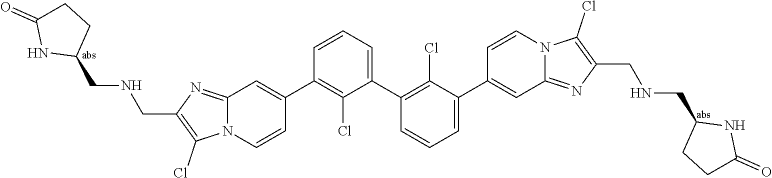 [SMILES: O=C1CC[C@@H](CNCc2nc3cc(-c4cccc(-c5cccc(-c6ccn7c(Cl)c(CNC[C@@H]8CCC(=O)N8)nc7c6)c5Cl)c4Cl)ccn3c2Cl)N1]

A mixture of 3-chloro-7-[2-chloro-3-[2-chloro-3-(3-chloro-2-formyl-imidazo[1,2-a]pyridin-7-yl)phenyl]phenyl]imidazo[1,2-a]pyridine-2-carbaldehyde (10 mg, 0.02 mmol), (5S)-5-(aminomethyl)pyrrolidin-2-one (8 mg, 0.07 mmol) and acetic acid (2 uL, 2.1 mg, 0.03 mmol) in MeOH/THF (1:1) was stirred for 1.5 hour at rt. NaBH₃CN (4.3 mg, 0.07 mmol) was added, the mixture was stirred for additional 20 min, quenched with water and directly purified by reversed phase chromatography to give (5S)-5-[[[3-chloro-7-[2-chloro-3-[2-chloro-3-[3-chloro-2-[[[(2S)-5-oxopyrrolidin-2-yl]methylamino]methyl]imidazo[1,2-a]pyridin-7-yl]phenyl]phenyl]imidazo[1,2-a]pyridin-2-yl]methylamino]methyl]pyrrolidin-2-one (10 mg, 74.7%). MS: m/z found 775.2 [M+H]⁺. ¹H NMR (400 MHz, Methanol-d₄): δ 8.38 (dd, J=7.1, 0.9 Hz, 2H), 7.66 (t, J=1.3 Hz, 2H), 7.61-7.52 (m, 4H), 7.46 (dd, J=6.4, 2.9 Hz, 2H), 7.26 (dd, J=7.1, 1.6 Hz, 2H), 4.10-3.99 (m, 4H), 3.90-3.80 (m, 2H), 2.88-2.71 (m, 4H), 2.37-2.23 (m, 6H), 1.83 (tt, J=12.6, 6.9 Hz, 2H).

### Example 438: N-(2-Aminoethyl)-6-[2-chloro-3-[2-chloro-3-[6-methoxy-5-[[[(2S)-5-oxopyrrolidin-2-yl]methylamino]methyl]-2-pyridyl]phenyl]phenyl]imidazo[1,2-b]pyridazine-2-carboxamide

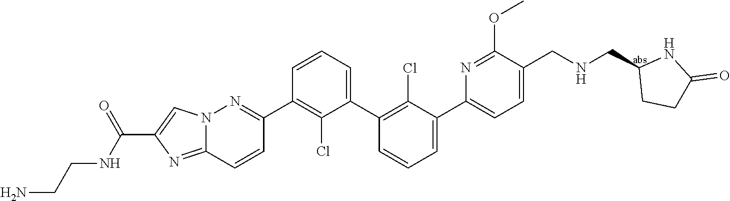 [SMILES: COc1nc(-c2cccc(-c3cccc(-c4ccc5nc(C(=O)NCCN)cn5n4)c3Cl)c2Cl)ccc1CNC[C@@H]1CCC(=O)N1]

### 6-[3-[3-[5-[[tert-Butoxycarbonyl-[[(2S)-5-oxopyrrolidin-2-yl]methyl]amino]methyl]-6-methoxy-2-pyridyl]-2-chloro-phenyl]-2-chloro-phenyl]imidazo[1,2-b]pyridazine-2-carboxylic acid

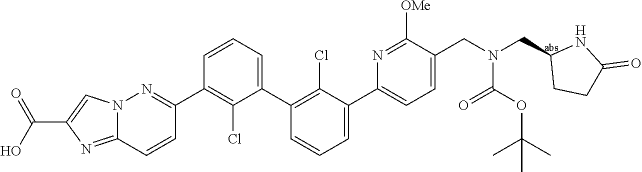 [SMILES: COc1nc(-c2cccc(-c3cccc(-c4ccc5nc(C(=O)O)cn5n4)c3Cl)c2Cl)ccc1CN(C[C@@H]1CCC(=O)N1)C(=O)OC(C)(C)C]

### tert-Butyl N-[[6-[3-[3-[2-(2-aminoethylcarbamoyl)imidazo[1,2-b]pyridazin-6-yl]-2-chloro-phenyl]-2-chloro-phenyl]-2-methoxy-3-pyridyl]methyl]-N-[[(2S)-5-oxopyrrolidin-2-yl]methyl]carbamate

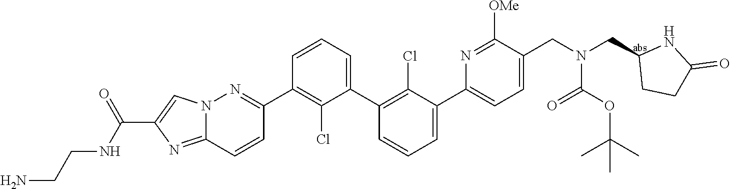 [SMILES: COc1nc(-c2cccc(-c3cccc(-c4ccc5nc(C(=O)NCCN)cn5n4)c3Cl)c2Cl)ccc1CN(C[C@@H]1CCC(=O)N1)C(=O)OC(C)(C)C]

To a mixture of 6-[3-[3-[5-[[tert-butoxycarbonyl-[[(2S)-5-oxopyrrolidin-2-yl]methyl]amino]methyl]-6-methoxy-2-pyridyl]-2-chloro-phenyl]-2-chloro-phenyl]imidazo[1,2-b]pyridazine-2-carboxylic acid (25 mg, 0.03 mmol) and HATU (13 mg, 0.03 mmol) in DMF was added DIPEA (6 uL, 4.5 mg, 0.03 mmol), followed by the addition of ethane-1,2-diamine (12 uL, 10.47 mg, 0.17 mmol). The mixture was stirred for 2 hours. Water was added and the residue was extracted into EtOAc and DCM three times. The combined organic layers were dried over anhydrous sodium sulfate, filtered, concentrated, and used in the next step. MS: m/z found 759.3 [M+H]⁺.

### N-(2-Aminoethyl)-6-[2-chloro-3-[2-chloro-3-[6-methoxy-5-[[[(2S)-5-oxopyrrolidin-2-yl]methylamino]methyl]-2-pyridyl]phenyl]phenyl]imidazo[1,2-b]pyridazine-2-carboxamide

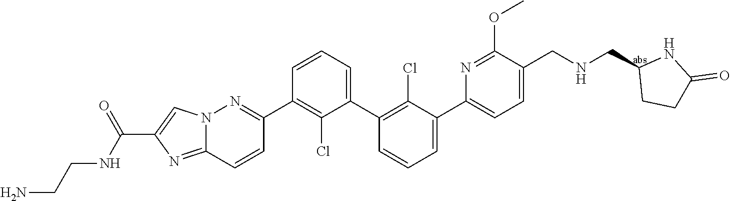 [SMILES: COc1nc(-c2cccc(-c3cccc(-c4ccc5nc(C(=O)NCCN)cn5n4)c3Cl)c2Cl)ccc1CNC[C@@H]1CCC(=O)N1]

A solution of tert-butyl N-[[6-[3-[3-[2-(2-aminoethylcarbamoyl)imidazo[1,2-b]pyridazin-6-yl]-2-chloro-phenyl]-2-chloro-phenyl]-2-methoxy-3-pyridyl]methyl]-N-[[(2S)-5-oxopyrrolidin-2-yl]methyl]carbamate (20 mg, 0.03 mmol) in DCM/TFA (2:1) was stirred for 30 minutes, concentrated in vacuo and purified by reversed phase chromatography to give N-(2-aminoethyl)-6-[2-chloro-3-[2-chloro-3-[6-methoxy-5-[[[(2S)-5-oxopyrrolidin-2-yl]methylamino]methyl]-2-pyridyl]phenyl]phenyl]imidazo[1,2-b]pyridazine-2-carboxamide (17 mg, 97.9%). MS: m/z found 659.2 [M+H]⁺. ¹H NMR (400 MHz, Methanol-d₄): δ 8.64 (d, J=0.7 Hz, 1H), 8.17-8.09 (m, 1H), 7.74 (d, J=7.5 Hz, 1H), 7.70 (dd, J=7.6, 1.9 Hz, 1H), 7.66 (dd, J=7.7, 1.7 Hz, 1H), 7.63-7.57 (m, 2H), 7.56-7.48 (m, 2H), 7.40 (dd, J=7.6, 1.7 Hz, 1H), 7.25 (d, J=7.4 Hz, 1H), 4.02 (s, 3H), 3.90-3.79 (m, 3H), 3.72 (t, J=5.8 Hz, 2H), 3.19 (t, J=5.8 Hz, 2H), 2.78-2.66 (m, 2H), 2.38-2.24 (m, 3H), 1.86-1.76 (m, 1H).

### Example 439: N-[(2S)-2-Aminopropyl]-6-[2-chloro-3-[2-chloro-3-[6-methoxy-5-[[[(2S)-5-oxopyrrolidin-2-yl]methylamino]methyl]-2-pyridyl]phenyl]phenyl]imidazo[1,2-b]pyridazine-2-carboxamide

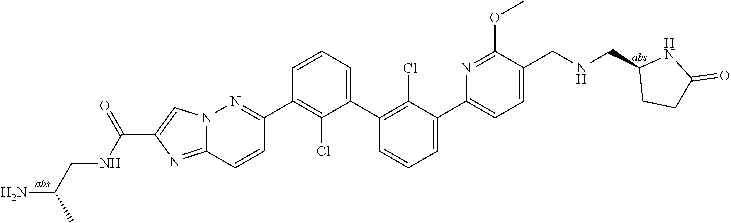 [SMILES: COc1nc(-c2cccc(-c3cccc(-c4ccc5nc(C(=O)NC[C@H](C)N)cn5n4)c3Cl)c2Cl)ccc1CNC[C@@H]1CCC(=O)N1]

### tert-Butyl N-[[6-[3-[3-[2-[[(2S)-2-(tert-butoxycarbonylamino)propyl]carbamoyl]imidazo[1,2-b]pyridazin-6-yl]-2-chloro-phenyl]-2-chloro-phenyl]-2-methoxy-3-pyridyl]methyl]-N-[[(2S)-5-oxopyrrolidin-2-yl]methyl]carbamate

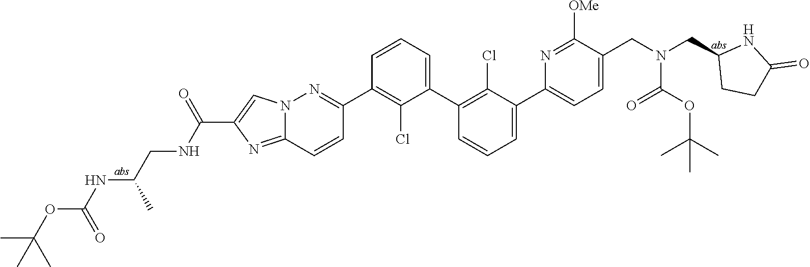 [SMILES: COc1nc(-c2cccc(-c3cccc(-c4ccc5nc(C(=O)NC[C@H](C)NC(=O)OC(C)(C)C)cn5n4)c3Cl)c2Cl)ccc1CN(C[C@@H]1CCC(=O)N1)C(=O)OC(C)(C)C]

To a mixture of 6-[3-[3-[5-[[tert-butoxycarbonyl-[[(2S)-5-oxopyrrolidin-2-yl]methyl]amino]methyl]-6-methoxy-2-pyridyl]-2-chloro-phenyl]-2-chloro-phenyl]imidazo[1,2-b]pyridazine-2-carboxylic acid (25 mg, 0.03 mmol) and HATU (13 mg, 0.03 mmol) in DMF was added DIPEA (6.1 uL, 4.5 mg, 0.03 mmol), followed by the addition of tert-butyl N-[(1S)-2-amino-1-methyl-ethyl]carbamate (18 mg, 0.10 mmol). The mixture was stirred for 2 hours. Water was added and the residue was extracted into EtOAc and DCM three times. The combined organic layers were dried over anhydrous sodium sulfate, filtered, concentrated, and used in the next step. MS: m/z found 873.3 [M+H]⁺.

### N-[(2S)-2-Aminopropyl]-6-[2-chloro-3-[2-chloro-3-[6-methoxy-5-[[[(2S)-5-oxopyrrolidin-2-yl]methylamino]methyl]-2-pyridyl]phenyl]phenyl]imidazo[1,2-b]pyridazine-2-carboxamide

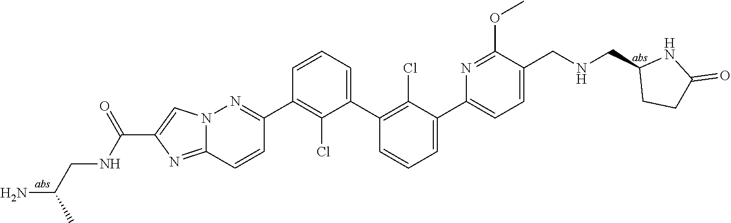 [SMILES: COc1nc(-c2cccc(-c3cccc(-c4ccc5nc(C(=O)NC[C@H](C)N)cn5n4)c3Cl)c2Cl)ccc1CNC[C@@H]1CCC(=O)N1]

A solution of tert-butyl N-[[6-[3-[3-[2-[[(2S)-2-(tert-butoxycarbonylamino)propyl]carbamoyl]imidazo[1,2-b]pyridazin-6-yl]-2-chloro-phenyl]-2-chloro-phenyl]-2-methoxy-3-pyridyl]methyl]-N-[[(2S)-5-oxopyrrolidin-2-yl]methyl]carbamate (20 mg, 0.02 mmol) in DCM/TFA (2:1) was stirred for 30 minutes, concentrated in vacuo and purified by reversed phase chromatography to give N-[(2S)-2-aminopropyl]-6-[2-chloro-3-[2-chloro-3-[6-methoxy-5-[[[(2S)-5-oxopyrrolidin-2-yl]methylamino]methyl]-2-pyridyl]phenyl]phenyl]imidazo[1,2-b]pyridazine-2-carboxamide (15.00 mg, 97.3%). MS: m/z found 673.2 [M+H]⁺. ¹H NMR (400 MHz, Methanol-d₄): δ 8.64 (d, J=0.7 Hz, 1H), 8.14 (dd, J=9.6, 0.7 Hz, 1H), 7.74 (d, J=7.4 Hz, 1H), 7.70 (dd, J=7.6, 1.8 Hz, 1H), 7.66 (dd, J=7.7, 1.7 Hz, 1H), 7.62-7.57 (m, 2H), 7.56-7.53 (m, 1H), 7.50 (t, J=7.6 Hz, 1H), 7.40 (dd, J=7.6, 1.7 Hz, 1H), 7.25 (d, J=7.4 Hz, 1H), 4.02 (s, 3H), 3.89-3.79 (m, 3H), 3.62 (d, J=5.7 Hz, 2H), 3.53 (h, J=6.7 Hz, 1H), 2.78-2.64 (m, 2H), 2.38-2.24 (m, 3H), 1.87-1.76 (m, 1H), 1.36 (d, J=6.5 Hz, 3H).

### Example 440: 6-[2-Chloro-3-[2-chloro-3-[6-methoxy-5-[[[(2S)-5-oxopyrrolidin-2-yl]methylamino]methyl]-2-pyridyl]phenyl]phenyl]-N-[2-(1H-imidazol-4-yl)ethyl]imidazo[1,2-b]pyridazine-2-carboxamide

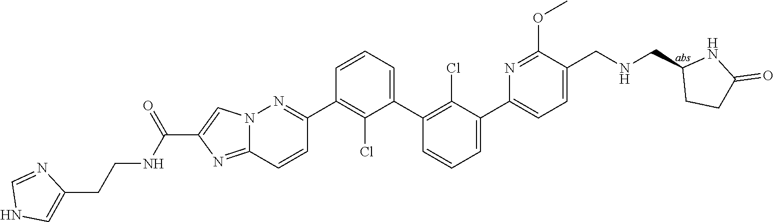 [SMILES: COc1nc(-c2cccc(-c3cccc(-c4ccc5nc(C(=O)NCCc6c[nH]cn6)cn5n4)c3Cl)c2Cl)ccc1CNC[C@@H]1CCC(=O)N1]

### tert-Butyl N-[[6-[2-chloro-3-[2-chloro-3-[2-[2-(1H-imidazol-4-yl)ethylcarbamoyl]imidazo[1,2-b]pyridazin-6-yl]phenyl]phenyl]-2-methoxy-3-pyridyl]methyl]-N-[[(2S)-5-oxopyrrolidin-2-yl]methyl]carbamate

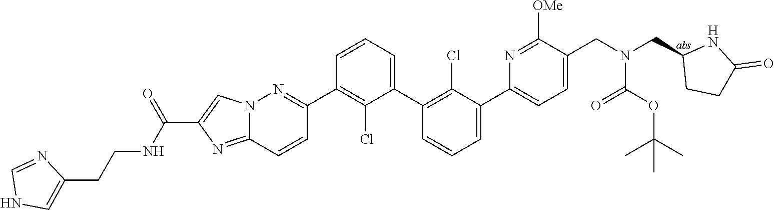 [SMILES: COc1nc(-c2cccc(-c3cccc(-c4ccc5nc(C(=O)NCCc6c[nH]cn6)cn5n4)c3Cl)c2Cl)ccc1CN(C[C@@H]1CCC(=O)N1)C(=O)OC(C)(C)C]

To a mixture of 6-[3-[3-[5-[[tert-butoxycarbonyl-[[(2S)-5-oxopyrrolidin-2-yl]methyl]amino]methyl]-6-methoxy-2-pyridyl]-2-chloro-phenyl]-2-chloro-phenyl]imidazo[1,2-b]pyridazine-2-carboxylic acid (20 mg, 0.03 mmol) and HATU (11 mg, 0.03 mmol) in DMF was added DIPEA (5 uL, 3.6 mg, 0.03 mmol), followed by the addition of 2-(1H-imidazol-4-yl)ethanamine (9 mg, 0.08 mmol). The mixture was stirred for 2 hours. Water was added and the residue was extracted into EtOAc and DCM three times. The combined organic layers were dried over anhydrous sodium sulfate, filtered, concentrated, and used in the next step. MS: m/z found 810.3 [M+H]⁺.

### 6-[2-Chloro-3-[2-chloro-3-[6-methoxy-5-[[[(2S)-5-oxopyrrolidin-2-yl]methylamino]methyl]-2-pyridyl]phenyl]phenyl]-N-[2-(1H-imidazol-4-yl)ethyl]imidazo[1,2-b]pyridazine-2-carboxamide

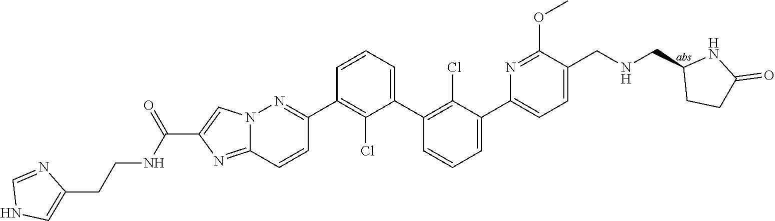 [SMILES: COc1nc(-c2cccc(-c3cccc(-c4ccc5nc(C(=O)NCCc6c[nH]cn6)cn5n4)c3Cl)c2Cl)ccc1CNC[C@@H]1CCC(=O)N1]

A solution of tert-butyl N-[[6-[2-chloro-3-[2-chloro-3-[2-[2-(1H-imidazol-4-yl)ethylcarbamoyl]imidazo[1,2-b]pyridazin-6-yl]phenyl]phenyl]-2-methoxy-3-pyridyl]methyl]-N-[[(2S)-5-oxopyrrolidin-2-yl]methyl]carbamate (15 mg, 0.02 mmol) in DCM/TFA (2:1) was stirred for 30 minutes, concentrated in vacuo and purified by reversed phase chromatography to give 6-[2-chloro-3-[2-chloro-3-[6-methoxy-5-[[[(2S)-5-oxopyrrolidin-2-yl]methylamino]methyl]-2-pyridyl]phenyl]phenyl]-N-[2-(1H-imidazol-4-yl)ethyl]imidazo[1,2-b]pyridazine-2-carboxamide (13 mg, 98.9%). MS: m/z found 710.2 [M+H]⁺. ¹H NMR (400 MHz, Methanol-d₄): δ 8.58 (d, J=0.7 Hz, 1H), 8.11 (dd, J=9.5, 0.7 Hz, 1H), 7.84 (d, J=1.2 Hz, 1H), 7.78 (d, J=7.5 Hz, 1H), 7.69 (dd, J=7.6, 1.8 Hz, 1H), 7.66 (dd, J=7.7, 1.7 Hz, 1H), 7.62-7.48 (m, 4H), 7.41 (dd, J=7.6, 1.7 Hz, 1H), 7.29 (d, J=7.5 Hz, 1H), 7.01 (d, J=1.1 Hz, 1H), 4.04 (s, 3H), 3.97 (m, 2H), 3.90 (m, 1H), 3.71 (t, J=7.1 Hz, 2H), 2.97 (t, J=7.1 Hz, 2H), 2.91-2.79 (m, 2H), 2.40-2.27 (m, 3H), 1.90-1.79 (m, 1H).

### Example 444: (5S)-5-[[[6-[2-Chloro-3-[2-chloro-3-[1-methyl-3-[[[(2S)-tetrahydrofuran-2-yl]methylamino]methyl]pyrrolo[2,3-b]pyridin-6-yl]phenyl]phenyl]-2-methoxy-3-pyridyl]methylamino]methyl]pyrrolidin-2-one

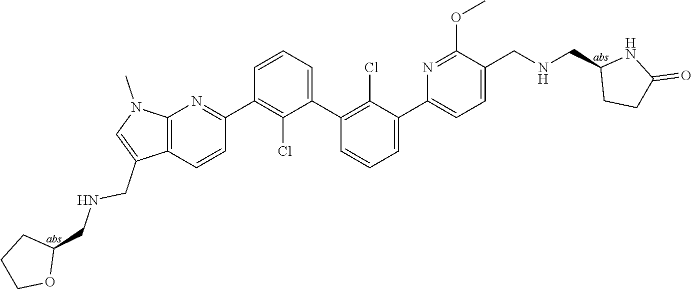 [SMILES: COc1nc(-c2cccc(-c3cccc(-c4ccc5c(CNC[C@@H]6CCCO6)cn(C)c5n4)c3Cl)c2Cl)ccc1CNC[C@@H]1CCC(=O)N1]

### tert-Butyl N-[[6-[2-chloro-3-[2-chloro-3-[1-methyl-3-[[[(2S)-tetrahydrofuran-2-yl]methylamino]methyl]pyrrolo[2,3-b]pyridin-6-yl]phenyl]phenyl]-2-methoxy-3-pyridyl]methyl]-N-[[(2S)-5-oxopyrrolidin-2-yl]methyl]carbamate

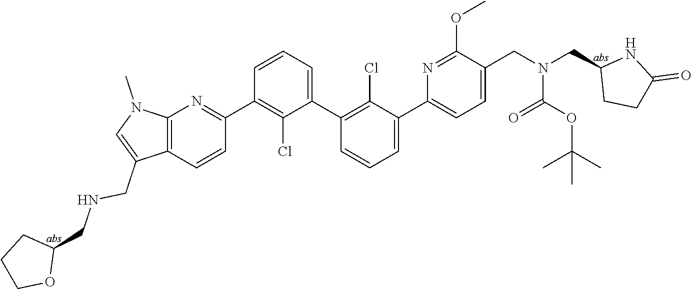 [SMILES: COc1nc(-c2cccc(-c3cccc(-c4ccc5c(CNC[C@@H]6CCCO6)cn(C)c5n4)c3Cl)c2Cl)ccc1CN(C[C@@H]1CCC(=O)N1)C(=O)OC(C)(C)C]

A mixture of tert-butyl N-[[6-[2-chloro-3-[2-chloro-3-(3-formyl-1-methyl-pyrrolo[2,3-b]pyridin-6-yl)phenyl]phenyl]-2-methoxy-3-pyridyl]methyl]-N-[[(2S)-5-oxopyrrolidin-2-yl]methyl]carbamate (40 mg, 0.06 mmol), [(2S)-tetrahydrofuran-2-yl]methanamine (11 mg, 0.11 mmol) and acetic acid (3.2 uL, 3.4 mg, 0.06 mmol) in MeOH/THF (1:1) was stirred for 1.5 hour at rt. NaBH₃CN (7 mg, 0.11 mmol) was added, the mixture was stirred for 30 min, quenched with water and directly purified by reversed phase chromatography to give tert-butyl N-[[6-[2-chloro-3-[2-chloro-3-[1-methyl-3-[[[(2S)-tetrahydrofuran-2-yl]methylamino]methyl]pyrrolo[2,3-b]pyridin-6-yl]phenyl]phenyl]-2-methoxy-3-pyridyl]methyl]-N-[[(2S)-5-oxopyrrolidin-2-yl]methyl]carbamate (30 mg, 67.0%). MS: m/z found 799.3 [M+H]⁺.

### (5S)-5-[[[6-[2-Chloro-3-[2-chloro-3-[1-methyl-3-[[[(2S)-tetrahydrofuran-2-yl]methylamino]methyl]pyrrolo[2,3-b]pyridin-6-yl]phenyl]phenyl]-2-methoxy-3-pyridyl]methylamino]methyl]pyrrolidin-2-one

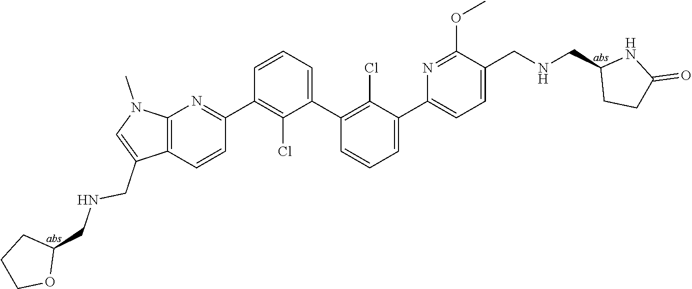 [SMILES: COc1nc(-c2cccc(-c3cccc(-c4ccc5c(CNC[C@@H]6CCCO6)cn(C)c5n4)c3Cl)c2Cl)ccc1CNC[C@@H]1CCC(=O)N1]

A solution of tert-butyl N-[[6-[2-chloro-3-[2-chloro-3-[1-methyl-3-[[[(2S)-tetrahydrofuran-2-yl]methylamino]methyl]pyrrolo[2,3-b]pyridin-6-yl]phenyl]phenyl]-2-methoxy-3-pyridyl]methyl]-N-[[(2S)-5-oxopyrrolidin-2-yl]methyl]carbamate (25 mg, 0.03 mmol) in DCM/TFA (2:1) was stirred for 30 minutes, concentrated in vacuo and purified by reversed phase chromatography to give (5S)-5-[[[6-[2-chloro-3-[2-chloro-3-[1-methyl-3-[[[(2S)-tetrahydrofuran-2-yl]methylamino]methyl]pyrrolo[2,3-b]pyridin-6-yl]phenyl]phenyl]-2-methoxy-3-pyridyl]methylamino]methyl]pyrrolidin-2-one (21 mg, 96.0%). MS: m/z found 699.3 [M+H]⁺. ¹H NMR (400 MHz, Methanol-d₄): δ 8.20 (d, J=8.1 Hz, 1H), 7.73 (d, J=7.5 Hz, 1H), 7.63 (dt, J=7.7, 1.9 Hz, 2H), 7.55 (s, 1H), 7.55-7.51 (m, 1H), 7.51-7.46 (m, 1H), 7.44-7.36 (m, 3H), 7.25 (d, J=7.4 Hz, 1H), 4.27 (s, 2H), 4.16-4.08 (m, 1H), 4.02 (s, 3H), 3.92 (s, 3H), 3.90-3.75 (m, 5H), 3.00 (dd, J=12.5, 3.3 Hz, 1H), 2.87 (dd, J=12.5, 9.2 Hz, 1H), 2.76-2.62 (m, 2H), 2.37-2.20 (m, 3H), 2.08 (dq, J=12.4, 6.9 Hz, 1H), 1.98-1.89 (m, 2H), 1.82 (ddd, J=17.9, 12.1, 7.0 Hz, 1H), 1.58 (dq, J=12.2, 7.5 Hz, 1H).

### Example 445: (5S)-5-[[[6-[2-Chloro-3-[2-chloro-3-[3-[[[(2S)-2-methoxypropyl]amino]methyl]-1-methyl-pyrrolo[2,3-b]pyridin-6-yl]phenyl]phenyl]-2-methoxy-3-pyridyl]methylamino]methyl]pyrrolidin-2-one

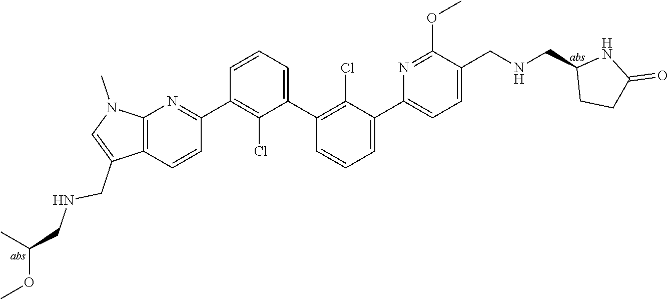 [SMILES: COc1nc(-c2cccc(-c3cccc(-c4ccc5c(CNC[C@H](C)OC)cn(C)c5n4)c3Cl)c2Cl)ccc1CNC[C@@H]1CCC(=O)N1]

### tert-Butyl N-[[6-[2-chloro-3-[2-chloro-3-[3-[[[(2S)-2-methoxypropyl]amino]methyl]-1-methyl-pyrrolo[2,3-b]pyridin-6-yl]phenyl]phenyl]-2-methoxy-3-pyridyl]methyl]-N-[[(2S)-5-oxopyrrolidin-2-yl]methyl]carbamate

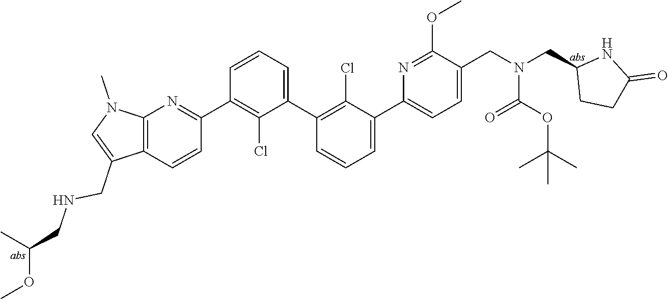 [SMILES: COc1nc(-c2cccc(-c3cccc(-c4ccc5c(CNC[C@H](C)OC)cn(C)c5n4)c3Cl)c2Cl)ccc1CN(C[C@@H]1CCC(=O)N1)C(=O)OC(C)(C)C]

A mixture of tert-butyl N-[[6-[2-chloro-3-[2-chloro-3-(3-formyl-1-methyl-pyrrolo[2,3-b]pyridin-6-yl)phenyl]phenyl]-2-methoxy-3-pyridyl]methyl]-N-[[(2S)-5-oxopyrrolidin-2-yl]methyl]carbamate (40 mg, 0.06 mmol), (2S)-2-methoxypropan-1-amine (10 mg, 0.11 mmol) and acetic acid (3.2 uL, 3.4 mg, 0.06 mmol) in MeOH/THF (1:1) was stirred for 1.5 hour at rt. NaBH₃CN (7 mg, 0.11 mmol) was added, the mixture was stirred for 30 min, quenched with water and directly purified by reversed phase chromatography to give tert-butyl N-[[6-[2-chloro-3-[2-chloro-3-[3-[[[(2S)-2-methoxypropyl]amino]methyl]-1-methyl-pyrrolo[2,3-b]pyridin-6-yl]phenyl]phenyl]-2-methoxy-3-pyridyl]methyl]-N-[[(2S)-5-oxopyrrolidin-2-yl]methyl]carbamate (30 mg, 68.0%). MS: m/z found 787.3 [M+H]⁺.

### (5S)-5-[[[6-[2-Chloro-3-[2-chloro-3-[3-[[[(2S)-2-methoxypropyl]amino]methyl]-1-methyl-pyrrolo[2,3-b]pyridin-6-yl]phenyl]phenyl]-2-methoxy-3-pyridyl]methylamino]methyl]pyrrolidin-2-one

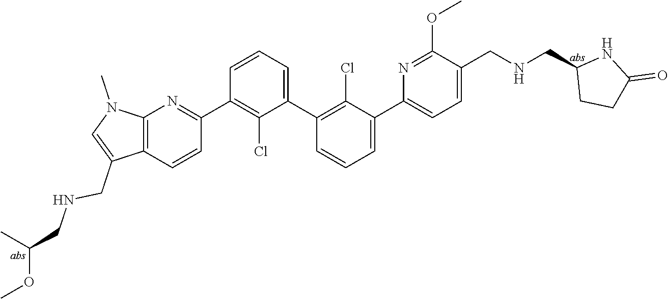 [SMILES: COc1nc(-c2cccc(-c3cccc(-c4ccc5c(CNC[C@H](C)OC)cn(C)c5n4)c3Cl)c2Cl)ccc1CNC[C@@H]1CCC(=O)N1]

A solution of tert-butyl N-[[6-[2-chloro-3-[2-chloro-3-[3-[[[(2S)-2-methoxypropyl]amino]methyl]-1-methyl-pyrrolo[2,3-b]pyridin-6-yl]phenyl]phenyl]-2-methoxy-3-pyridyl]methyl]-N-[[(2S)-5-oxopyrrolidin-2-yl]methyl]carbamate (25 mg, 0.03 mmol) in DCM/TFA (2:1) was stirred for 30 minutes, concentrated in vacuo and purified by reversed phase chromatography to give (5S)-5-[[[6-[2-chloro-3-[2-chloro-3-[3-[[[(2S)-2-methoxypropyl]amino]methyl]-1-methyl-pyrrolo[2,3-b]pyridin-6-yl]phenyl]phenyl]-2-methoxy-3-pyridyl]methylamino]methyl]pyrrolidin-2-one (21 mg, 96.2%). MS: m/z found 687.3 [M+H]⁺. ¹H NMR (400 MHz, Methanol-d₄): δ 8.21 (d, J=8.1 Hz, 1H), 7.73 (d, J=7.5 Hz, 1H), 7.64 (dt, J=7.6, 1.7 Hz, 2H), 7.58 (s, 1H), 7.51 (dt, J=14.0, 7.6 Hz, 2H), 7.46-7.36 (m, 3H), 7.25 (d, J=7.4 Hz, 1H), 4.28 (s, 2H), 4.02 (s, 3H), 3.92 (s, 3H), 3.89-3.76 (m, 3H), 3.62 (ddd, J=9.4, 6.2, 3.2 Hz, 1H), 3.37 (s, 3H), 2.99 (d, J=12.4 Hz, 1H), 2.86 (dd, J=12.6, 9.3 Hz, 1H), 2.77-2.63 (m, 2H), 2.40-2.21 (m, 3H), 1.88-1.74 (m, 1H), 1.17 (d, J=6.1 Hz, 3H).

### Example 452: (5S)-5-[[[6-[2-Chloro-3-[2-chloro-3-[1-methyl-3-[[[(2S)-5-oxopyrrolidin-2-yl]methylamino]methyl]indol-6-yl]phenyl]phenyl]-2-methoxy-3-pyridyl]methylamino]methyl]pyrrolidin-2-one

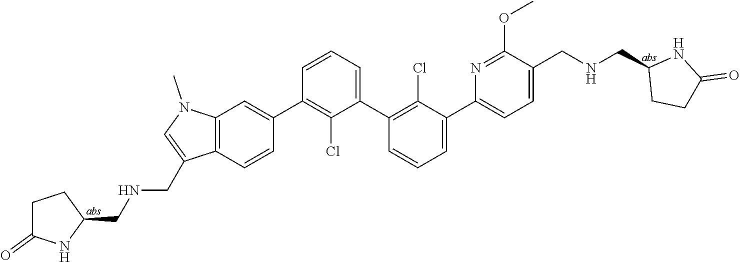 [SMILES: COc1nc(-c2cccc(-c3cccc(-c4ccc5c(CNC[C@@H]6CCC(=O)N6)cn(C)c5c4)c3Cl)c2Cl)ccc1CNC[C@@H]1CCC(=O)N1]

### 6-Bromo-1-methyl-indole-3-carbaldehyde

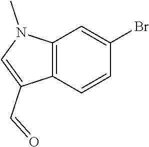

To mixture of 6-bromo-1H-indole-3-carbaldehyde (0.50 g, 2.23 mmol) and K₂CO₃ (0.46 g, 3.35 mmol) in DMF (5 mL) were added Mel (0.19 mL, 0.44 g, 3.12 mmol). The mixture was stirred at ambient temperature for 2 hrs. After addition of water, the reaction was extracted with EtOAc three times, and the organic layer was washed with a brine solution, and dried over Na₂SO₄. The solvents were removed in vacuo, and the residue was purified by flash column chromatography (EtOAc in hexanes, 0 to 20% gradient) to afford 6-bromo-1-methyl-indole-3-carbaldehyde (0.53 g, 98.8%). MS: m/z found 238.0 [M+H]⁺. ¹H NMR (400 MHz, Chloroform-d): δ 9.91 (s, 111), 8.12 (d, J=8.4 Hz, 111), 7.59 (s, 111), 7.46 (d, J=1.6 Hz, 111), 7.39 (dd, J=8.4, 1.7 Hz, 1H), 3.79 (s, 3H).

### 6-[2-Chloro-3-[2-chloro-3-(5-formyl-6-methoxy-2-pyridyl)phenyl]phenyl]-1-methyl-indole-3-carbaldehyde

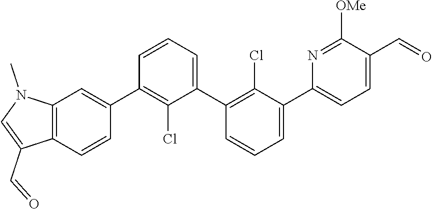 [SMILES: COc1nc(-c2cccc(-c3cccc(-c4ccc5c(C=O)cn(C)c5c4)c3Cl)c2Cl)ccc1C=O]

To a solution of 6-bromo-1-methyl-indole-3-carbaldehyde (30 mg, 0.13 mmol), 6-[2-chloro-3-[2-chloro-3-(4,4,5,5-tetramethyl-1,3,2-dioxaborolan-2-yl)phenyl]phenyl]-2-methoxy-pyridine-3-carbaldehyde (61 mg, 0.13 mmol) and potassium carbonate (52 mg, 0.38 mmol) in degassed dioxane/water (6:1) was added Pd(PPh₃)₄ (15 mg, 0.01 mmol) and the mixture was stirred at 95° C. for 1 hour under N₂(g). Water was added and the mixture was extracted into EtOAc three times. The combined organic layers were dried over anhydrous sodium sulfate, concentrated, and purified on silica gel column using EtOAc in hexanes (0 to 40% gradient) to provide 6-[2-chloro-3-[2-chloro-3-(5-formyl-6-methoxy-2-pyridyl)phenyl]phenyl]-1-methyl-indole-3-carbaldehyde (56 mg, 86.2%). MS: m/z found 515.1 [M+H]⁺.

### (5S)-5-[[[6-[2-Chloro-3-[2-chloro-3-[1-methyl-3-[[[(2S)-5-oxopyrrolidin-2-yl]methylamino]methyl]indol-6-yl]phenyl]phenyl]-2-methoxy-3-pyridyl]methylamino]methyl]pyrrolidin-2-one

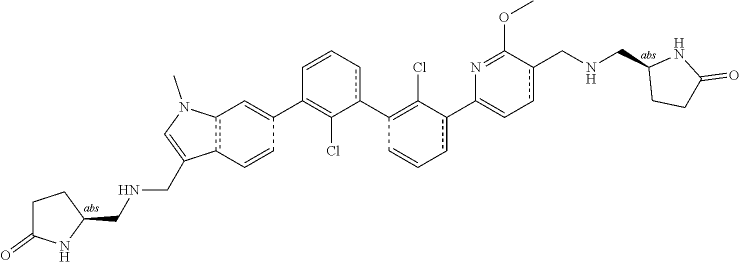

A mixture of 6-[2-chloro-3-[2-chloro-3-(5-formyl-6-methoxy-2-pyridyl)phenyl]phenyl]-1-methyl-indole-3-carbaldehyde (50 mg, 0.10 mmol), (5S)-5-(aminomethyl)pyrrolidin-2-one (44 mg, 0.39 mmol) and acetic acid (11 uL, 12 mg, 0.19 mmol) in MeOH/THF (1:1) was stirred for 1.5 hour at rt. NaBH₃CN (24 mg, 0.39 mmol) was added, the mixture was stirred for additional 20 min, quenched with water and directly purified by reversed phase chromatography to give (5S)-5-[[[6-[2-chloro-3-[2-chloro-3-[1-methyl-3-[[[(2S)-5-oxopyrrolidin-2-yl]methylamino]methyl]indol-6-yl]phenyl]phenyl]-2-methoxy-3-pyridyl]methylamino]methyl]pyrrolidin-2-one (63 mg, 91.2%). MS: m/z found 711.3 [M+H]⁺. ¹H NMR (400 MHz, Methanol-d₄): δ 7.75 (d, J=4.5 Hz, 1H), 7.74-7.71 (m, 1H), 7.62 (dd, J=7.7, 1.7 Hz, 1H), 7.51-7.43 (m, 4H), 7.37 (dd, J=7.6, 1.8 Hz, 1H), 7.36 (s, 1H), 7.32 (dd, J=6.9, 2.3 Hz, 1H), 7.27-7.21 (m, 2H), 4.29-4.17 (m, 2H), 4.02 (s, 3H), 3.93-3.88 (m, 1H), 3.87-3.79 (m, 6H), 3.01-2.89 (m, 2H), 2.79-2.64 (m, 2H), 2.40-2.23 (m, 6H), 1.89-1.75 (m, 2H).

### Example 453: 8-[2-Chloro-3-[2-chloro-3-[1-methyl-3-[[[(2S)-5-oxopyrrolidin-2-yl]methylamino]methyl]indol-6-yl]phenyl]phenyl]-3-[[[(2S)-5-oxopyrrolidin-2-yl]methylamino]methyl]pyrido[1,2-a]pyrimidin-4-one

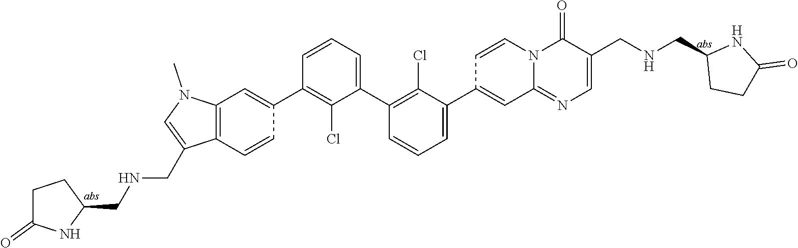

### 8-[2-Chloro-3-[2-chloro-3-(3-formyl-1-methyl-indol-6-yl)phenyl]phenyl]-4-oxo-pyrido[1,2-a]pyrimidine-3-carbaldehyde

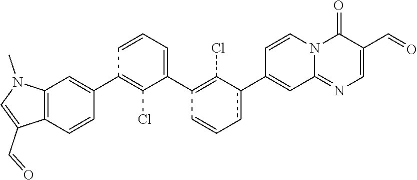

To a solution of 6-bromo-1-methyl-indole-3-carbaldehyde (30 mg, 0.13 mmol), 8-[2-chloro-3-[2-chloro-3-(4,4,5,5-tetramethyl-1,3,2-dioxaborolan-2-yl)phenyl]phenyl]-4-oxo-pyrido[1,2-a]pyrimidine-3-carbaldehyde (66 mg, 0.13 mmol) and potassium carbonate (52 mg, 0.38 mmol) in degassed dioxane/water (6:1) was added Pd(PPh₃)₄ (15 mg, 0.01 mmol) and the mixture was stirred at 95° C. for 1 h under N₂(g). Water was added and the mixture was extracted into EtOAc three times. The combined organic layers were dried over anhydrous sodium sulfate, concentrated, and purified on silica gel column using MeOH in DCM (0 to 5% gradient) to provide 8-[2-chloro-3-[2-chloro-3-(3-formyl-1-methyl-indol-6-yl)phenyl]phenyl]-4-oxo-pyrido[1,2-a]pyrimidine-3-carbaldehyde (55 mg, 79.0%). MS: m/z found 552.1 [M+H]⁺.

### 8-[2-Chloro-3-[2-chloro-3-[1-methyl-3-[[[(2S)-5-oxopyrrolidin-2-yl]methylamino]methyl]indol-6-yl]phenyl]phenyl]-3-[[[(2S)-5-oxopyrrolidin-2-yl]methylamino]methyl]pyrido[1,2-a]pyrimidin-4-one

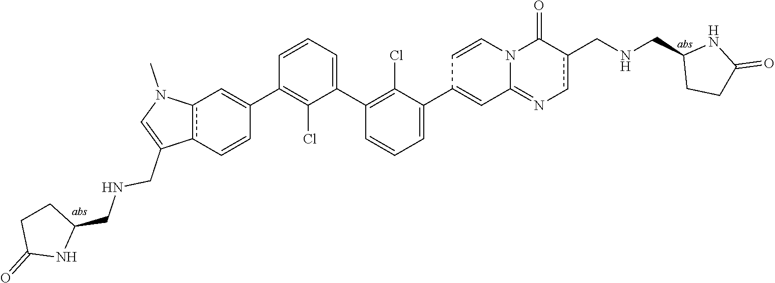

A mixture of 8-[2-chloro-3-[2-chloro-3-(3-formyl-1-methyl-indol-6-yl)phenyl]phenyl]-4-oxo-pyrido[1,2-a]pyrimidine-3-carbaldehyde (30 mg, 0.05 mmol), (5S)-5-(aminomethyl)pyrrolidin-2-one (25 mg, 0.22 mmol) and acetic acid (6.1 uL, 6.5 mg, 0.11 mmol) in MeOH/THF (1:1) was stirred for 1.5 hour at rt. NaBH₃CN (14 mg, 0.22 mmol) was added, the mixture was stirred for additional 20 min, quenched with water and directly purified by reversed phase chromatography to give 8-[2-chloro-3-[2-chloro-3-[1-methyl-3-[[[(2S)-5-oxopyrrolidin-2-yl]methylamino]methyl]indol-6-yl]phenyl]phenyl]-3-[[[(2S)-5-oxopyrrolidin-2-yl]methylamino]methyl]pyrido[1,2-a]pyrimidin-4-one (37 mg, 91.0%). MS: m/z found 748.3 [M+H]⁺. ¹H NMR (400 MHz, Methanol-d₄): δ 9.15 (d, J=7.3 Hz, 1H), 8.50 (s, 1H), 8.43 (s, 1H), 7.81-7.74 (m, 2H), 7.65-7.57 (m, 2H), 7.57-7.46 (m, 5H), 7.43 (s, 1H), 7.36 (dd, J=6.7, 2.5 Hz, 1H), 7.27 (dd, J=8.2, 1.4 Hz, 1H), 4.42-4.29 (m, 2H), 3.99-3.87 (m, 4H), 3.87 (s, 3H), 3.13-3.03 (m, 2H), 2.86-2.71 (m, 2H), 2.42-2.23 (m, 6H), 1.91-1.78 (m, 2H).

### Example 80: (S)-8-(2,2′-Dichloro-3′-(6-methoxy-5-((((5-oxopyrrolidin-2-yl)methyl)amino)methyl)pyridin-2-yl)-[1,1′-biphenyl]-3-yl)-3-(((2-hydroxyethyl)amino)methyl)-4H-pyrido[1,2-a]pyrimidin-4-one

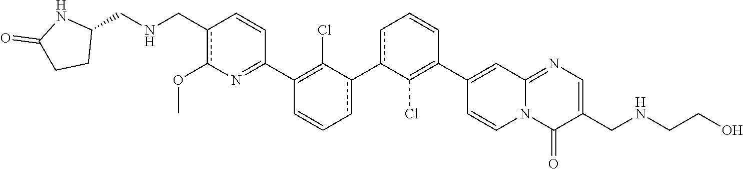

### (S)-5-((((6-(2,2′-Dichloro-3′-(4,4,5,5-tetramethyl-1,3,2-dioxaborolan-2-yl)-[1,1′-biphenyl]-3-yl)-2-methoxypyridin-3-yl)methyl)amino)methyl)pyrrolidin-2-one

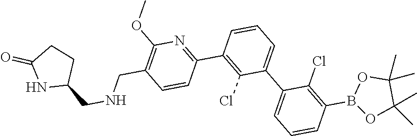

6-[2-chloro-3-[2-chloro-3-(4,4,5,5-tetramethyl-1,3,2-dioxaborolan-2-yl)phenyl]phenyl]-2-methoxy-pyridine-3-carbaldehyde (250 mg, 0.52 mmol), (5S)-5-(aminomethyl)-pyrrolidin-2-one (77 mg, 0.67 mmol) and acetic acid (47 mg, 0.77 mmol) were dissolved in 8 ml of 1:1 MeOH/THF solvent mix, and the reaction was stirred at 25° C. for 2 hours. NaBH₃CN (65 mg, 1.03 mmol), was then added portion-wise, and the resulting mixture was further stirred at 25° C. for 1 hour. The reaction was concentrated and the residue was dissolved in 10 ml water and the aqueous was extracted with EtOAc (3×10 ml). The combined organics were washed with 10 ml brine, dried over sodium sulfate, then filtered and concentrated to afford (5S)-5-[[[6-[2-chloro-3-[2-chloro-3-(4,4,5,5-tetramethyl-1,3,2-dioxaborolan-2-yl)phenyl]phenyl]-2-methoxy-3-pyridyl]methylamino]methyl]pyrrolidin-2-one (303 mg) as a white foam. Material was used as—is for the next step. MS: m/z found 582.4, 584 [M+H]⁺.

### tert-Butyl (S)-((6-(2,2′-dichloro-3′-(4,4,5,5-tetramethyl-1,3,2-dioxaborolan-2-yl)-[1,1′-biphenyl]-3-yl)-2-methoxypyridin-3-yl)-methyl)((5-oxopyrrolidin-2-yl)methyl)carbamate

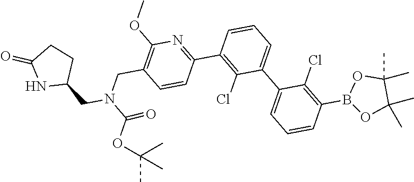

(5S)-5-[[[6-[2-chloro-3-[2-chloro-3-(4,4,5,5-tetramethyl-1,3,2-dioxaborolan-2-yl)phenyl]phenyl]-2-methoxy-3-pyridyl]methylamino]methyl]pyrrolidin-2-one (303 mg, 0.52 mmol) and DIEA (360 μL, 270 mg, 2.08 mmol) were dissolved in 10 ml anhydrous THE and di-tert-butyl dicarbonate, (227 mg, 1.04 mmol) was added portion-wise. The resulting mixture was stirred at rt for 12 hours. The reaction was diluted with EtOAc (20 ml) and the resulting solution was washed with aqueous saturated sodium bicarbonate solution (10 mL×2) and brine (15 mL×2). The organic layer was concentrated in vacuo to give a crude sample, which was purified by silica gel chromatography, eluting with 10-100% EtOAc gradient in hexane to afford tert-butyl N-[[6-[2-chloro-3-[2-chloro-3-(4,4,5,5-tetramethyl-1,3,2-dioxaborolan-2-yl)phenyl]phenyl]-2-methoxy-3-pyridyl]methyl]-N-[[(2S)-5-oxopyrrolidin-2-yl]methyl]carbamate (205 mg, 58% yield) as a white foam. MS: m/z found 682.4, 684.4 [M+H]⁺.

### tert-Butyl (S)-((6-(2,2′-dichloro-3′-(3-formyl-4-oxo-4H-pyrido[1,2-a]pyrimidin-8-yl)-[1,1′-biphenyl]-3-yl)-2-methoxypyridin-3-yl)methyl)((5-oxopyrrolidin-2-yl)methyl)carbamate

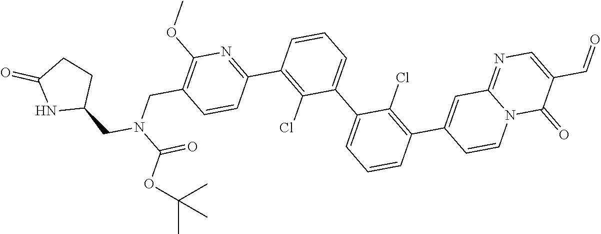 [SMILES: COc1nc(-c2cccc(-c3cccc(-c4ccn5c(=O)c(C=O)cnc5c4)c3Cl)c2Cl)ccc1CN(C[C@@H]1CCC(=O)N1)C(=O)OC(C)(C)C]

Pd(PPh₃)₄ (80 mg, 0.07 mmol), potassium carbonate (143 mg, 1.03 mmol), tert-butyl N-[[6-[2-chloro-3-[2-chloro-3-(4,4,5,5-tetramethyl-1,3,2-dioxaborolan-2-yl)phenyl]phenyl]-2-methoxy-3-pyridyl]methyl]-N-[[(2S)-5-oxopyrrolidin-2-yl]methyl]carbamate (294 mg, 0.34 mmol) and 8-bromo-4-oxo-pyrido[1,2-a]pyrimidine-3-carbaldehyde (105 mg, 0.41 mmol) were suspended in 10 ml of 4:1 dioxane/water solvent mix, and the reaction was heated at 105° C. for 20 minutes. The solution was subsequently cooled to rt, diluted with 25 ml water, and extracted with EtOAc (3×20 mL). The combined organics were concentrated and crude sample was purified by silica chromatography, eluting with 0-3% MeOH gradient in DCM to afford tert-butyl N-[[6-[2-chloro-3-[2-chloro-3-(3-formyl-4-oxo-pyrido[1,2-a]pyrimidin-8-yl)phenyl]phenyl]-2-methoxy-3-pyridyl]-methyl]-N-[[(2S)-5-oxopyrrolidin-2-yl]methyl]carbamate (228 mg, 90.8% yield) as a yellow foam. MS: m/z found 728.3, 730.3 [M+H]⁺.

### tert-Butyl (S)-((6-(2,2′-dichloro-3′-(3-(((2-hydroxyethyl)amino)methyl)-4-oxo-4H-pyrido[1,2-a]pyrimidin-8-yl)-[1,1′-biphenyl]-3-yl)-2-methoxypyridin-3-yl)methyl)((5-oxopyrrolidin-2-yl)methyl)carbamate

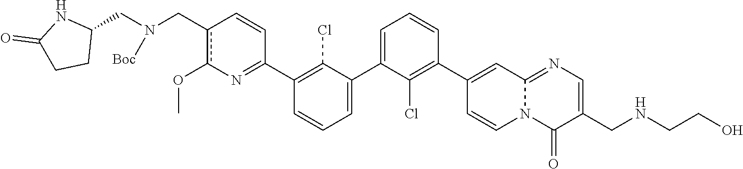

tert-Butyl N-[[6-[2-chloro-3-[2-chloro-3-(3-formyl-4-oxo-pyrido[1,2-a]pyrimidin-8-yl)phenyl]phenyl]-2-methoxy-3-pyridyl]methyl]-N-[[(2S)-5-oxopyrrolidin-2-yl]methyl]carbamate (19 mg, 0.03 mmol), 2-aminoethanol (5 mg, 0.08 mmol) and acetic acid (3 mg, 0.05 mmol) were dissolved in 1 ml of 1:1 MeOH/THF solvent mix, and the reaction was stirred at 25° C. for 3 hours. NaBH₃CN (3.3 mg, 0.05 mmol) was added portion-wise, and the resulting mixture was stirred at 25° C. for 1 hour. The reaction was concentrated and the residue was dissolved in water (3 mL), then extracted with DCM (3×2 mL). The combined organics were further washed with brine, dried over sodium sulfate, then filtered and concentrated to afford tert-butyl N-[[6-[2-chloro-3-[2-chloro-3-[3-[(2-hydroxyethylamino)methyl]-4-oxo-pyrido[1,2-a]pyrimidin-8-yl]phenyl]phenyl]-2-methoxy-3-pyridyl]methyl]-N-[[(2S)-5-oxopyrrolidin-2-yl]methyl]carbamate (23 mg) as an orange oil. Material was used as—is for the next step. MS: m/z found 773.4, 775.4 [M+H]⁺.

### (S)-8-(2,2′-Dichloro-3′-(6-methoxy-5-((((5-oxopyrrolidin-2-yl)methyl)amino)methyl)pyridin-2-yl)-[1,1′-biphenyl]-3-yl)-3-(((2-hydroxyethyl)amino)methyl)-4H-pyrido[1,2-a]pyrimidin-4-one

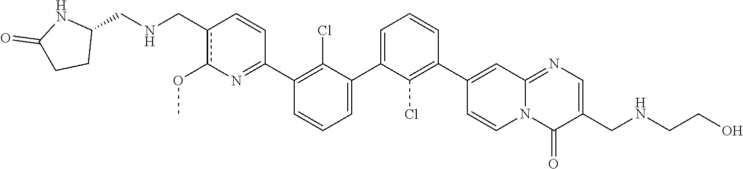

tert-Butyl N-[[6-[2-chloro-3-[2-chloro-3-[3-[(2-hydroxyethylamino)methyl]-4-oxo-pyrido[1,2-a]pyrimidin-8-yl]phenyl]phenyl]-2-methoxy-3-pyridyl]methyl]-N-[[(2S)-5-oxopyrrolidin-2-yl]methyl]carbamate (23 mg, 0.03 mmol) was dissolved in 0.5 ml DCM and 4M HCl solution in dioxane (22 μL, 0.09 mmol) was added. The reaction was stirred at rt for 20 minutes and the mixture was subsequently concentrated. Crude residue was purified by reverse phase HPLC to afford 8-[2-chloro-3-[2-chloro-3-[6-methoxy-5-[[[(2S)-5-oxopyrrolidin-2-yl]methylamino]methyl]-2-pyridyl]phenyl]phenyl]-3-[(2-hydroxyethylamino)methyl]pyrido[1,2-a]pyrimidin-4-one (7 mg, 35.0% yield) as a light yellow foam. MS: m/z found 673.4, 675.4 [M+H]⁺. ¹H NMR (400 MHz, Chloroform-d) δ 9.08 (d, J=7.5 Hz, 1H), 8.36 (s, 1H), 7.76 (d, J=1.9 Hz, 1H), 7.71-7.66 (m, 1H), 7.59 (d, J=7.5 Hz, 1H), 7.51-7.42 (m, 4H), 7.38 (dd, J=7.5, 1.9 Hz, 1H), 7.33 (dd, J=7.6, 1.7 Hz, 1H), 7.28 (s, 1H), 6.01 (d, J=5.5 Hz, 1H), 4.03 (d, J=1.3 Hz, 3H), 3.92 (s, 2H), 3.81 (s, 3H), 3.74-3.70 (m, 2H), 2.89-2.84 (m, 2H), 2.80 (dd, J=12.0, 4.2 Hz, 1H), 2.57 (dd, J=12.0, 8.6 Hz, 1H), 2.36 (t, J=8.1 Hz, 2H), 2.29-2.19 (m, 1H), 1.77 (dd, J=13.4, 6.2 Hz, 1H).

### Example 206: 8-(2,2′-Dichloro-3′-(6-methoxy-5-(((((S)-5-oxopyrrolidin-2-yl)methyl)amino)methyl)pyridin-2-yl)-[1,1′-biphenyl]-3-yl)-3-((((6-oxopiperidin-2-yl)methyl)amino)methyl)-4H-pyrido[1,2-a]pyrimidin-4-one

Compound was prepared in the same manner as described for Example 80, utilizing intermediate (S)-tert-butyl ((6-(2,2′-dichloro-3′-(3-formyl-4-oxo-4H-pyrido[1,2-a]pyrimidin-8-yl)-[1,1′-biphenyl]-3-yl)-2-methoxypyridin-3-yl)methyl)((5-oxopyrrolidin-2-yl)methyl)carbamate. 2-aminoethan-1-ol was replaced by 6-(aminomethyl)-piperidin-2-one.

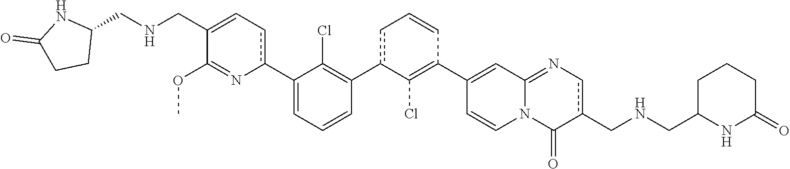

White solid. MS: m/z found 740.35, 742 [M+H]⁺. ¹H NMR formic acid salt (400 MHz, Methanol-d₄) δ 9.15 (d, J=7.5 Hz, 1H), 8.49 (s, 1H), 8.43 (s, 1H), 7.81-7.71 (m, 2H), 7.68-7.58 (m, 3H), 7.57-7.48 (m, 3H), 7.40 (dd, J=7.5, 1.7 Hz, 1H), 7.26 (d, J=7.4 Hz, 1H), 4.03 (s, 3H), 3.96-3.83 (m, 5H), 3.59 (s, 1H), 2.89-2.68 (m, 4H), 2.38-2.25 (m, 5H), 1.93 (s, 2H), 1.87-1.68 (m, 2H), 1.48 (q, J=10.7, 9.5 Hz, 1H).

### Example 207: (S)-8-(2,2′-Dichloro-3′-(6-methoxy-5-((((5-oxopyrrolidin-2-yl)methyl)amino)methyl)pyridin-2-yl)-[1,1′-biphenyl]-3-yl)-3-(((2-(2-oxoimidazolidin-1-yl)ethyl)amino)methyl)-4H-pyrido[1,2-a]pyrimidin-4-one

Compound was prepared in the same manner as described for Example 80, utilizing intermediate (S)-tert-butyl ((6-(2,2′-dichloro-3′-(3-formyl-4-oxo-4H-pyrido[1,2-a]pyrimidin-8-yl)-[1,1′-biphenyl]-3-yl)-2-methoxypyridin-3-yl)methyl)((5-oxopyrrolidin-2-yl)methyl)carbamate. 2-aminoethan-1-ol was replaced by 1-(2-aminoethyl)-imidazolidin-2-one.

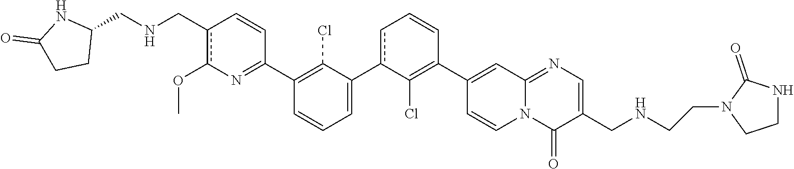

White solid. MS: m/z found 741.4, 743 [M+H]⁺. ¹H NMR formic acid salt (400 MHz, Methanol-d₄) δ 9.14 (d, J=7.4 Hz, 1H), 8.53 (s, 1H), 8.45 (s, 1H), 7.80 (s, 1H), 7.74 (d, J=7.6 Hz, 1H), 7.69-7.55 (m, 4H), 7.54-7.47 (m, 2H), 7.39 (d, J=7.8 Hz, 1H), 7.24 (d, J=7.4 Hz, 1H), 4.07-3.97 (m, 5H), 3.89-3.78 (m, 3H), 3.53-3.45 (m, 2H), 3.44-3.34 (m, 4H), 2.99 (t, J=6.1 Hz, 2H), 2.77-2.63 (m, 2H), 2.38-2.22 (m, 3H), 1.81 (d, J=13.1 Hz, 1H).

### Example 217: (S)-5-((((6-(3′-(3-Amino-2-(hydroxymethyl)-thieno[3,2-b]-pyridin-6-yl)-2,2′-dichloro-[1,1′-biphenyl]-3-yl)-2-methoxypyridin-3-yl)methyl)amino)methyl)pyrrolidin-2-one

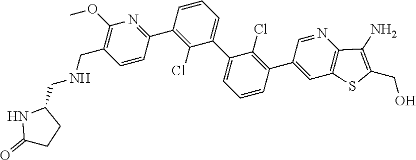 [SMILES: COc1nc(-c2cccc(-c3cccc(-c4cnc5c(N)c(CO)sc5c4)c3Cl)c2Cl)ccc1CNC[C@@H]1CCC(=O)N1]

### (3-Amino-6-bromothieno[3,2-b]-pyridin-2-yl)-methanol

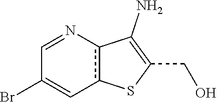

Methyl 3-amino-6-bromo-thieno[3,2-b]-pyridine-2-carboxylate (200 mg, 0.70 mmol) was dissolved in 4 mL THE and the solution was cooled to −10° C. lithium aluminum hydride (56 mg, 1.60 mmol) was added portion-wise under a nitrogen stream over 2 minutes, and the resulting mixture was stirred at −10° C. for 1 hour. Reaction was then gradually warmed to rt over 3 hours. Disodium sulfate decahydrate (200 mg) was then added, along with 15 mL MeOH, and the reaction was stirred overnight. Reaction was filtered and the solids were further washed with EtOAc (2×20 mL). The filtrate was concentrated under reduced pressure and the crude solid was suspended in 1 mL MeOH. The precipitate was filtered and further washed with hexane, then dried to afford (3-amino-6-bromo-thieno[3,2-b]-pyridin-2-yl)-methanol (57 mg, 31.6% yield) as green solid. MS: m/z found 260.9 [M+H]⁺.

### 6-(3′-(3-Amino-2-(hydroxymethyl)thieno[3,2-b]pyridin-6-yl)-2,2′-dichloro-[1,1′-biphenyl]-3-yl)-2-methoxynicotinaldehyde

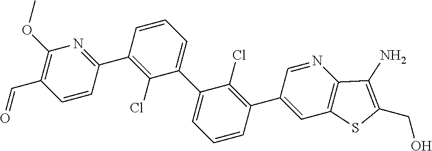 [SMILES: COc1nc(-c2cccc(-c3cccc(-c4cnc5c(N)c(CO)sc5c4)c3Cl)c2Cl)ccc1C=O]

Pd(PPh₃)₄ (19 mg, 0.02 mmol), potassium carbonate (34 mg, 0.25 mmol), 6-[2-chloro-3-[2-chloro-3-(4,4,5,5-tetramethyl-1,3,2-dioxaborolan-2-yl)phenyl]phenyl]-2-methoxy-pyridine-3-carbaldehyde (40 mg, 0.08 mmol), and (3-amino-6-bromo-thieno[3,2-b]pyridin-2-yl)methanol (28 mg, 0.11 mmol) were suspended in 1 mL of 4:1 dioxane/water solvent mixture, then the solution was heated at 105° C. for 20 minutes. Reaction was cooled to rt, diluted with 3 mL water, and extracted with EtOAc (3×2 mL). The combined organics were concentrated and crude sample was purified by normal phase SiO₂ chromatography (5-100% EtOAc/hexane) to afford 6-[3-[3-[3-amino-2-(hydroxymethyl)thieno[3,2-b]pyridin-6-yl]-2-chloro-phenyl]-2-chloro-phenyl]-2-methoxy-pyridine-3-carbaldehyde (20 mg, 27.1% yield) as a brown solid. MS: m/z found 536.2, 538 [M+H]⁺.

### (S)-5-((((6-(3′-(3-Amino-2-(hydroxymethyl)-thieno[3,2-b]-pyridin-6-yl)-2,2′-dichloro-[1,1′-biphenyl]-3-yl)-2-methoxypyridin-3-yl)methyl)amino)methyl)pyrrolidin-2-one

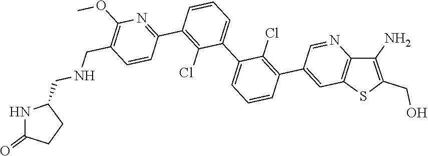 [SMILES: COc1nc(-c2cccc(-c3cccc(-c4cnc5c(N)c(CO)sc5c4)c3Cl)c2Cl)ccc1CNC[C@@H]1CCC(=O)N1]

6-[3-[3-[3-Amino-2-(hydroxymethyl)thieno[3,2-b]pyridin-6-yl]-2-chloro-phenyl]-2-chloro-phenyl]-2-methoxy-pyridine-3-carbaldehyde (20 mg, 0.02 mmol), (5S)-5-(aminomethyl)pyrrolidin-2-one (8 mg, 0.07 mmol), and acetic acid (2 mg, 0.03 mmol) were dissolved in 1 mL of 1:1 MeOH/THF solvent mix, then the solution was stirred at 25° C. for 2 hours. NaBH₃CN (2.8 mg, 0.04 mmol) was added and the resulting mixture was stirred at 25° C. for 1 hour. Reaction was subsequently diluted with water (2 mL) and extracted with EtOAc (3×2 mL). The combined organics were further washed with brine, dried over sodium sulfate, filtered, and concentrated under reduced pressure. Crude sample was purified by reverse phase HPLC to afford (5S)-5-[[[6-[3-[3-[3-amino-2-(hydroxymethyl)thieno[3,2-b]pyridin-6-yl]-2-chloro-phenyl]-2-chloro-phenyl]-2-methoxy-3-pyridyl]methylamino]methyl]pyrrolidin-2-one (2.2 mg, 15.5% yield) as a yellow solid. MS: m/z found 634.35, 636 [M+H]⁺. ¹H NMR (400 MHz, Chloroform-d) δ 8.76-8.68 (m, 1H), 8.22-8.15 (m, 1H), 7.67 (d, J=7.6 Hz, 1H), 7.58 (d, J=7.4 Hz, 1H), 7.47-7.40 (m, 3H), 7.39-7.32 (m, 2H), 7.28 (s, 1H), 6.11 (s, 1H), 4.90 (d, J=1.3 Hz, 2H), 4.02 (d, J=1.2 Hz, 3H), 3.80 (d, J=14.5 Hz, 3H), 2.80 (dd, J=12.1, 4.0 Hz, 1H), 2.64-2.53 (m, 1H), 2.35 (q, J=8.5 Hz, 2H), 2.24 (dq, J=14.8, 7.3 Hz, 1H), 0.95-0.78 (m, 2H).

### Example 218: (S)-5-((((6-(2,2′-Dichloro-3′-(2,3,4,5-tetrahydropyrido[3,2-f][1,4]oxazepin-8-yl)-[1,1′-biphenyl]-3-yl)-2-methoxypyridin-3-yl)methyl)amino)methyl)887yrrolidine-2-one

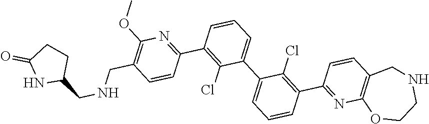

### tert-Butyl (S)-8-(3′-(5-(((tert-butoxycarbonyl)((5-oxopyrrolidin-2-yl)methyl)amino)methyl)-6-methoxypyridin-2-yl)-2,2′-dichloro-[1,1′-biphenyl]-3-yl)-2,3-dihydropyrido[3,2-f][1,4]oxazepine-4(5H)-carboxylate

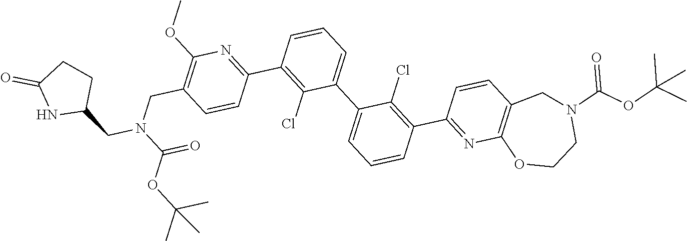

Pd(PPh₃)₄ (12 mg, 0.01 mmol), potassium carbonate (21 mg, 0.15 mmol), tert-butyl N-[[6-[2-chloro-3-[2-chloro-3-(4,4,5,5-tetramethyl-1,3,2-dioxaborolan-2-yl)phenyl]phenyl]-2-methoxy-3-pyridyl]methyl]-N-[[(2S)-5-oxopyrrolidin-2-yl]methyl]carbamate (35 mg, 0.05 mmol), and tert-butyl 8-chloro-3,5-dihydro-2H-pyrido[3,2-f][1,4]oxazepine-4-carboxylate (19 mg, 0.07 mmol) were suspended in 1 mL of 4:1 dioxane/water solvent mixture, then the solution was heated at 105° C. for 1 hour. Reaction was cooled to rt, diluted with 3 mL water, and extracted with EtOAc (3×2 mL). The combined organics were concentrated and crude sample was purified by normal phase SiO₂ chromatography (10-100% EtOAc/hexane) to afford tert-butyl 8-[3-[3-[5-[[tert-butoxycarbonyl-[[(2S)-5-oxopyrrolidin-2-yl]methyl]amino]methyl]-6-methoxy-2-pyridyl]-2-chloro-phenyl]-2-chloro-phenyl]-3,5-dihydro-2H-pyrido[3,2-f][1,4]oxazepine-4-carboxylate (26.3 mg, 63.7% yield) as a clear oil. MS: m/z found 804.4, 806 [M+H]⁺.

### (S)-5-((((6-(2,2′-Dichloro-3′-(2,3,4,5-tetrahydropyrido[3,2-f][1,4]oxazepin-8-yl)-[1,1′-biphenyl]-3-yl)-2-methoxypyridin-3-yl)methyl)amino)methyl)pyrrolidin-2-one

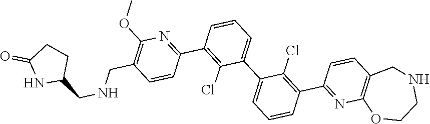

tert-Butyl 8-[3-[3-[5-[[tert-butoxycarbonyl-[[(2S)-5-oxopyrrolidin-2-yl]methyl]amino]methyl]-6-methoxy-2-pyridyl]-2-chloro-phenyl]-2-chloro-phenyl]-3,5-dihydro-2H-pyrido[3,2-f][1,4]oxazepine-4-carboxylate (26 mg, 0.03 mmol) was dissolved in 0.5 mL DCM and 4M HCl solution in dioxane (33 μL, 5 mg, 0.13 mmol) was added. Reaction was stirred at rt for 20 minutes and solvent was removed under reduced pressure. Crude sample was purified by reverse phase HPLC to afford (5S)-5-[[[6-[2-chloro-3-[2-chloro-3-(2,3,4,5-tetrahydropyrido[3,2-f][1,4]oxazepin-8-yl)phenyl]phenyl]-2-methoxy-3-pyridyl]methylamino]methyl]pyrrolidin-2-one (12 mg, 61.2% yield) as a white solid. MS: m/z found 604.3, 606 [M+H]⁺. ¹H NMR formic acid salt (400 MHz, Methanol-d₄) δ 8.45 (s, 1H), 7.89 (d, J=7.7 Hz, 1H), 7.79 (d, J=7.5 Hz, 1H), 7.64 (dd, J=7.7, 1.6 Hz, 1H), 7.56-7.46 (m, 3H), 7.45-7.39 (m, 2H), 7.37 (dd, J=7.6, 1.7 Hz, 1H), 7.28 (d, J=7.5 Hz, 1H), 4.33 (t, J=4.6 Hz, 2H), 4.20 (s, 2H), 4.04 (d, J=5.3 Hz, 5H), 3.92 (d, J=7.6 Hz, 1H), 3.42 (t, J=4.5 Hz, 2H), 2.93 (q, J=7.0 Hz, 2H), 2.41-2.27 (m, 3H), 1.91-1.79 (m, 1H).

### Example 235: (5S)-5-((((6-(2,2′-Dichloro-3′-(5-((((S)-5-oxopyrrolidin-2-yl)methyl)amino)-5,6,7,8-tetrahydroquinolin-2-yl)-[1,1′-biphenyl]-3-yl)-2-methoxypyridin-3-yl)methyl)amino)methyl)pyrrolidin-2-one (racemic)

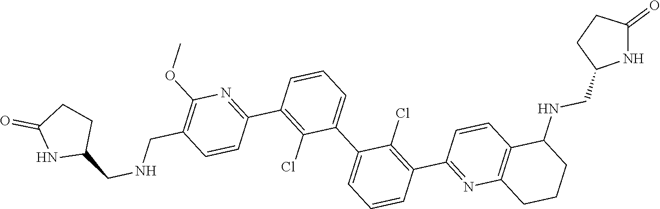

### (5S)-5-(((2-Chloro-5,6,7,8-tetrahydroquinolin-5-yl)amino)-methyl)pyrrolidin-2-one

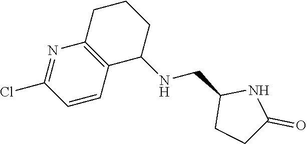 [SMILES: O=C1CC[C@@H](CNC2CCCc3nc(Cl)ccc32)N1]

2-Chloro-7,8-dihydro-6H-quinolin-5-one (200.00 mg, 0.88 mmol), (5S)-5-(aminomethyl)pyrrolidin-2-one (202 mg, 1.77 mmol), and tetraethoxytitanium (605 mg, 2.65 mmol) were suspended in 4 mL anhydrous THF. Reaction mixture was heated at 105° C. for 3 hours in a sealed tube. Reaction was cooled to −50° C. and NaBH₄ (100 mg, 2.65 mmol) was added portion-wise under a nitrogen stream. The mixture was warmed to rt over 1 hour. Reaction was subsequently added drop-wise to an ice water solution (15 mL) and the resulting slurry was filtered through CELITE®. The filtrate was extracted with EtOAc (3×10 mL) and the combined organics were further washed with brine (10 mL), dried over sodium sulfate, filtered, and concentrated under reduced pressure to afford (5S)-5-[[(2-chloro-5,6,7,8-tetrahydroquinolin-5-yl)amino]-methyl]pyrrolidin-2-one (280 mg, crude) as a brown oil. MS: m/z found 280, 282 [M+H]⁺. Material was used for the next step without further purification.

### tert-butyl (2-Chloro-5,6,7,8-tetrahydroquinolin-5-yl)(((S)-5-oxopyrrolidin-2-yl)methyl)carbamate (racemic)

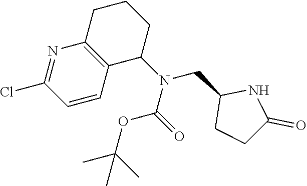 [SMILES: CC(C)(C)OC(=O)N(C[C@@H]1CCC(=O)N1)C1CCCc2nc(Cl)ccc21]

(5S)-5-[[(2-Chloro-5,6,7,8-tetrahydroquinolin-5-yl)amino]methyl]pyrrolidin-2-one (280 mg, 0.86 mmol), DMAP (20 mg), DIEA (600 μL, 445.63 mg, 3.45 mmol), and tert-butoxycarbonyl tert-butyl carbonate (377 mg, 1.73 mmol) were dissolved in 5 mL THF, then the mixture was stirred at rt for 12 hours. Reaction was diluted with EtOAc (10 mL) and the resulting solution was washed with saturated aqueous NaHCO₃(5 mL×2), followed by brine (5 mL×2). The organic layer was concentrated under reduced pressure and crude sample was purified by normal phase SiO₂ chromatography (10-100% EtOAc/hexane) to afford tert-butyl N-(2-chloro-5,6,7,8-tetrahydroquinolin-5-yl)-N-[[(2S)-5-oxopyrrolidin-2-yl]methyl]carbamate (200 mg, 54.6% yield) as a yellow oil. MS: m/z found 380 [M+H]⁺. ¹H NMR (400 MHz, Chloroform-d) δ 7.71 (d, J=8.1 Hz, 1H), 7.11 (dd, J=8.2, 3.8 Hz, 1H), 4.29-4.01 (m, 1H), 3.74 (dd, J=13.6, 6.7 Hz, 1H), 3.03-2.75 (m, 4H), 2.74-2.56 (m, 1H), 2.44 (ddd, J=17.6, 9.9, 2.9 Hz, 1H), 2.14 (ddt, J=19.5, 13.8, 6.4 Hz, 1H), 1.99 (dd, J=13.8, 9.8 Hz, 3H), 1.86-1.64 (m, 2H), 1.51 (dd, J=5.2, 1.3 Hz, 9H), 1.25 (td, J=7.1, 1.2 Hz, 1H).

### tert-Butyl ((6-(3′-(5-((tert-butoxycarbonyl)(((S)-5-oxopyrrolidin-2-yl)methyl)amino)-5,6,7,8-tetrahydroquinolin-2-yl)-2,2′-dichloro-[1,1′-biphenyl]-3-yl)-2-methoxypyridin-3-yl)methyl)(((S)-5-oxopyrrolidin-2-yl)methyl)carbamate (racemic)

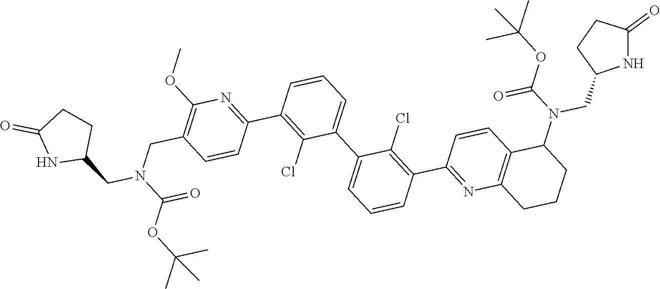

Pd(PPh₃)₄ (12 mg, 0.01 mmol), potassium carbonate (21 mg, 0.15 mmol), tert-butyl N-[[6-[2-chloro-3-[2-chloro-3-(4,4,5,5-tetramethyl-1,3,2-dioxaborolan-2-yl)phenyl]phenyl]-2-methoxy-3-pyridyl]methyl]-N-[[(2S)-5-oxopyrrolidin-2-yl]methyl]carbamate (35 mg, 0.05 mmol), and tert-butyl N-(2-chloro-5,6,7,8-tetrahydroquinolin-5-yl)-N-[[(2S)-5-oxopyrrolidin-2-yl]methyl]carbamate (28 mg, 0.07 mmol) were suspended in 1 mL of 4:1 dioxane/water solvent mixture, then the solution was heated at 105° C. for 20 minutes. Reaction was cooled to rt, diluted with 3 mL water, and extracted with EtOAc (3×2 mL). The combined organics were concentrated under reduced pressure and crude sample was purified by normal phase SiO₂ chromatography (0-4% MeOH/DCM) to afford tert-butyl N-[2-[3-[3-[5-[[tert-butoxycarbonyl-[[(2S)-5-oxopyrrolidin-2-yl]methyl]amino]methyl]-6-methoxy-2-pyridyl]-2-chloro-phenyl]-2-chloro-phenyl]-5,6,7,8-tetrahydroquinolin-5-yl]-N-[[(2S)-5-oxopyrrolidin-2-yl]methyl]carbamate (25.6 mg, 55.5% yield) as a clear film. MS: m/z found 899.5, 901 [M+H]⁺.

### (5S)-5-((((6-(2,2′-Dichloro-3′-(5-((((S)-5-oxopyrrolidin-2-yl)methyl)amino)-5,6,7,8-tetrahydroquinolin-2-yl)-[1,1′-biphenyl]-3-yl)-2-methoxypyridin-3-yl)methyl)amino)methyl)pyrrolidin-2-one (racemic)

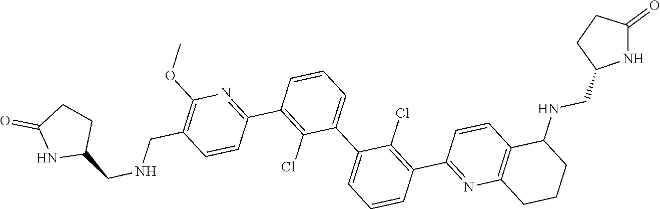

tert-Butyl N-[2-[3-[3-[5-[[tert-butoxycarbonyl-[[(2S)-5-oxopyrrolidin-2-yl]methyl]amino]methyl]-6-methoxy-2-pyridyl]-2-chloro-phenyl]-2-chloro-phenyl]-5,6,7,8-tetrahydroquinolin-5-yl]-N-[[(2S)-5-oxopyrrolidin-2-yl]methyl]carbamate (26 mg, 0.03 mmol) was dissolved in 1 mL DCM and 4M HCl solution in dioxane (28 μL, 4 mg, 0.11 mmol) was added. Reaction was stirred at rt for 20 minutes and solvent was removed under reduced pressure. Crude sample was purified by reverse phase HPLC to afford (5S)-5-[[[6-[2-chloro-3-[2-chloro-3-[5-[[(2S)-5-oxopyrrolidin-2-yl]methylamino]-5,6,7,8-tetrahydroquinolin-2-yl]phenyl]phenyl]-2-methoxy-3-pyridyl]methylamino]methyl]pyrrolidin-2-one (12.8 mg, 64.3% yield) as a white solid. MS: m/z found 699.35, 671 [M+H]⁺. ¹H NMR formic acid salt (400 MHz, Methanol-d₄) δ 8.42 (s, 1H), 8.01 (dd, J=8.1, 4.5 Hz, 1H), 7.82 (d, J=7.5 Hz, 1H), 7.64 (dd, J=7.7, 1.6 Hz, 1H), 7.55-7.48 (m, 3H), 7.48-7.35 (m, 3H), 7.30 (d, J=7.4 Hz, 1H), 4.12 (d, J=2.4 Hz, 2H), 4.06 (s, 3H), 4.04-3.93 (m, 2H), 3.83 (q, J=6.2 Hz, 1H), 3.07-2.87 (m, 5H), 2.84 (d, J=5.8 Hz, 1H), 2.44-2.24 (m, 6H), 2.15-2.00 (m, 2H), 1.99-1.80 (m, 4H).

### Example 237: (S)-8-(4″-((1-Aminocyclopropyl)methyl)-2,2′-dichloro-[1,1′:3′,1″-terphenyl]-3-yl)-3-((((5-oxopyrrolidin-2-yl)methyl)amino)methyl)-4H-pyrido[1,2-a]pyrimidin-4-one

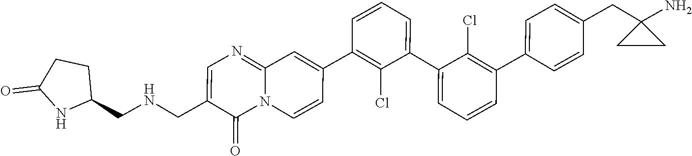 [SMILES: NC1(Cc2ccc(-c3cccc(-c4cccc(-c5ccn6c(=O)c(CNC[C@@H]7CCC(=O)N7)cnc6c5)c4Cl)c3Cl)cc2)CC1]

### (S)-8-(3′-Bromo-2,2′-dichloro-[1,1′-biphenyl]-3-yl)-3-((((5-oxopyrrolidin-2-yl)methyl)amino)methyl)-4H-pyrido[1,2-a]pyrimidin-4-one

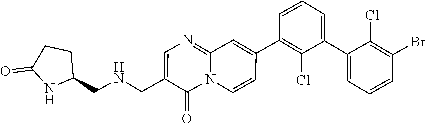 [SMILES: O=C1CC[C@@H](CNCc2cnc3cc(-c4cccc(-c5cccc(Br)c5Cl)c4Cl)ccn3c2=O)N1]

8-[3-(3-Bromo-2-chloro-phenyl)-2-chloro-phenyl]-4-oxo-pyrido[1,2-a]pyrimidine-3-carbaldehyde (300 mg, 0.62 mmol), (5S)-5-(aminomethyl)pyrrolidin-2-one (92 mg, 0.81 mmol), and acetic acid (55.81 mg, 0.93 mmol) were dissolved in MeOH/THF (1:1 (v v), 8 mL) and the solution was stirred at 25° C. for 2 hours. NaBH₃CN (78 mg, 1.24 mmol) was then added and the resulting mixture was stirred at 25° C. for 1 hour. Reaction was subsequently diluted with water (20 mL) and extracted with EtOAc (3×20 mL). The combined organics were further washed with brine (10 mL), dried over sodium sulfate, filtered and concentrated under reduced pressure to afford 8-[3-(3-bromo-2-chloro-phenyl)-2-chloro-phenyl]-3-[[[(2S)-5-oxopyrrolidin-2-yl]methylamino]methyl]pyrido[1,2-a]pyrimidin-4-one (325 mg) as a white foam. MS: m/z found 572, 574 [M+H]⁺. Material was used for the next step without further purification.

### tert-Butyl (S)-((8-(3′-bromo-2,2′-dichloro-[1,1′-biphenyl]-3-yl)-4-oxo-4H-pyrido[1,2-a]pyrimidin-3-yl)methyl)((5-oxopyrrolidin-2-yl)methyl)carbamate

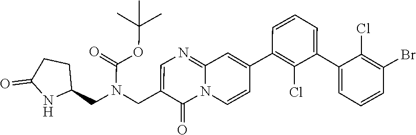 [SMILES: CC(C)(C)OC(=O)N(Cc1cnc2cc(-c3cccc(-c4cccc(Br)c4Cl)c3Cl)ccn2c1=O)C[C@@H]1CCC(=O)N1]

8-[3-(3-Bromo-2-chloro-phenyl)-2-chloro-phenyl]-3-[[[(2S)-5-oxopyrrolidin-2-yl]methylamino]-methyl]-pyrido[1,2-a]pyrimidin-4-one (325 mg, 0.57 mmol), DIEA (395 μL, 293. mg, 2.27 mmol), and tert-butoxycarbonyl tert-butyl carbonate (248 mg, 1.14 mmol) were dissolved in 6 mL THF, then the mixture was stirred at rt for 12 hours. Reaction was diluted with EtOAc (10 mL) and the resulting solution was washed with saturated aqueous NaHCO₃(5 mL×2), followed by brine (5 mL×2). The organic layer was concentrated under reduced pressure and crude sample was purified by normal phase SiO₂ chromatography (10-100% EtOAc/hexane) to afford tert-butyl N-[[8-[3-(3-bromo-2-chloro-phenyl)-2-chloro-phenyl]-4-oxo-pyrido[1,2-a]pyrimidin-3-yl]methyl]-N-[[(2S)-5-oxopyrrolidin-2-yl]methyl]carbamate (163 mg, 42.7% yield) as a yellow oil. MS: m/z found 673 [M+H]⁺.

### tert-Butyl (S)-((8-(2,2′-dichloro-3′-(4,4,5,5-tetramethyl-1,3,2-dioxaborolan-2-yl)-[1,1′-biphenyl]-3-yl)-4-oxo-4H-pyrido[1,2-a]pyrimidin-3-yl)methyl)((5-oxopyrrolidin-2-yl)methyl)carbamate

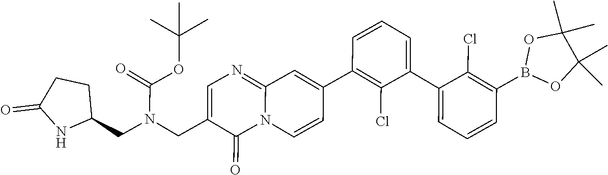 [SMILES: CC(C)(C)OC(=O)N(Cc1cnc2cc(-c3cccc(-c4cccc(B5OC(C)(C)C(C)(C)O5)c4Cl)c3Cl)ccn2c1=O)C[C@@H]1CCC(=O)N1]

tert-Butyl N-[[8-[3-(3-bromo-2-chloro-phenyl)-2-chloro-phenyl]-4-oxo-pyrido[1,2-a]pyrimidin-3-yl]methyl]-N-[[(2S)-5-oxopyrrolidin-2-yl]methyl]carbamate (163 mg, 0.24 mmol), 4,4,5,5-tetramethyl-2-(4,4,5,5-tetramethyl-1,3,2-dioxaborolan-2-yl)-1,3,2-dioxaborolane (80 mg, 0.32 mmol), 1,1′-bis(diphenylphosphino)-ferrocene-palladium(ii) dichloride dichloromethane complex (20 mg, 0.02 mmol), and potassium acetate (67 mg, 0.68 mmol) were suspended in 4 mL dioxane in a sealed tube. Nitrogen gas was bubbled through the reaction solution for 2 minutes, then the vessel was sealed and heated at 95° C. for 6 hours. Reaction was cooled to rt and the mixture was filtered through CELITE®. The filtrate was concentrated under reduced pressure and the crude oil was purified by normal phase SiO₂ chromatography (0-5% MeOH/DCM) to afford tert-butyl N-[[8-[2-chloro-3-[2-chloro-3-(4,4,5,5-tetramethyl-1,3,2-dioxaborolan-2-yl)phenyl]phenyl]-4-oxo-pyrido[1,2-a]pyrimidin-3-yl]methyl]-N-[[(2S)-5-oxopyrrolidin-2-yl]methyl]carbamate (188 mg, 86.2% yield) as a clear oil. MS: m/z found 719.45, 721 [M+H]⁺.

### tert-butyl (S)-((8-(4″-((1-Aminocyclopropyl)methyl)-2,2′-dichloro-[1,1′:3′,1″-terphenyl]-3-yl)-4-oxo-4H-pyrido[1,2-a]pyrimidin-3-yl)methyl)((5-oxopyrrolidin-2-yl)methyl)carbamate

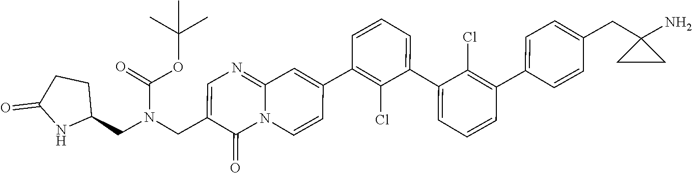 [SMILES: CC(C)(C)OC(=O)N(Cc1cnc2cc(-c3cccc(-c4cccc(-c5ccc(CC6(N)CC6)cc5)c4Cl)c3Cl)ccn2c1=O)C[C@@H]1CCC(=O)N1]

Pd(PPh₃)₄ (15 mg, 0.01 mmol), potassium carbonate (28 mg, 0.20 mmol), tert-butyl N-[[8-[2-chloro-3-[2-chloro-3-(4,4,5,5-tetramethyl-1,3,2-dioxaborolan-2-yl)phenyl]phenyl]-4-oxo-pyrido[1,2-a]pyrimidin-3-yl]methyl]-N-[[(2S)-5-oxopyrrolidin-2-yl]methyl]carbamate (60 mg, 0.07 mmol), and 1-[(4-bromophenyl)methyl]cyclopropanamine (20 mg, 0.09 mmol) were suspended in dioxane/water (4:1, 1 mL), then the solution was heated at 105° C. for 20 minutes. Reaction was cooled to rt, diluted with 2 mL water, and extracted with EtOAc (3×2 mL). The combined organics were concentrated under reduced pressure and crude sample was purified by reverse phase HPLC to afford tert-butyl N-[[8-[3-[3-[4-[(1-aminocyclopropyl)methyl]-phenyl]-2-chloro-phenyl]-2-chloro-phenyl]-4-oxo-pyrido[1,2-a]pyrimidin-3-yl]methyl]-N-[[(2S)-5-oxopyrrolidin-2-yl]methyl]carbamate (9.5 mg, 19.3% yield) as a clear oil. MS: m/z found 738, 740 [M+H]⁺.

### (S)-8-(4″-((1-Aminocyclopropyl)methyl)-2,2′-dichloro-[1,1′:3′,1″-terphenyl]-3-yl)-3-((((5-oxopyrrolidin-2-yl)methyl)amino)methyl)-4H-pyrido[1,2-a]pyrimidin-4-one

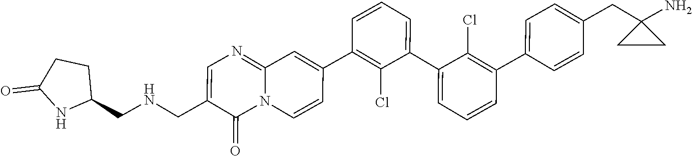 [SMILES: NC1(Cc2ccc(-c3cccc(-c4cccc(-c5ccn6c(=O)c(CNC[C@@H]7CCC(=O)N7)cnc6c5)c4Cl)c3Cl)cc2)CC1]

tert-Butyl N-[[8-[3-[3-[4-[(1-aminocyclopropyl)methyl]phenyl]-2-chloro-phenyl]-2-chloro-phenyl]-4-oxo-pyrido[1,2-a]pyrimidin-3-yl]methyl]-N-[[(2S)-5-oxopyrrolidin-2-yl]methyl]carbamate, (9.5 mg, 0.01 mmol) was dissolved in 0.5 mL DCM and 4M HCl solution in dioxane (13 μL, 0.05 mmol) was added. Reaction was stirred at rt for 20 minutes and solvent was removed under reduced pressure. Crude sample was purified by reverse phase HPLC to afford 8-[3-[3-[4-[(1-aminocyclopropyl)-methyl]-phenyl]-2-chloro-phenyl]-2-chloro-phenyl]-3-[[[(2S)-5-oxopyrrolidin-2-yl]-methylamino]methyl]pyrido[1,2-a]pyrimidin-4-one (3.7 mg, 45.1% yield) as a white foam. MS: m/z found 638.3, 640 [M+H]⁺. ¹H NMR formic acid salt (400 MHz, Methanol-d₄) δ 9.14 (d, J=7.4 Hz, 1H), 8.51 (s, 1H), 8.43 (s, 1H), 7.78 (s, 1H), 7.60 (d, J=8.2 Hz, 2H), 7.54 (d, J=7.4 Hz, 1H), 7.52-7.46 (m, 4H), 7.46-7.34 (m, 4H), 3.91 (dd, J=19.0, 5.9 Hz, 3H), 3.06 (s, 2H), 2.89-2.71 (m, 2H), 2.40-2.22 (m, 3H), 1.91-1.74 (m, 1H), 0.95 (s, 4H).

### Example 261: (S)-8-(2,2′-Dichloro-3′-(2,3,4,5-tetrahydropyrido[3,2-f][1,4]oxazepin-8-yl)-[1,1′-biphenyl]-3-yl)-3-((((5-oxopyrrolidin-2-yl)methyl)amino)methyl)-4H-pyrido[1,2-a]pyrimidin-4-one

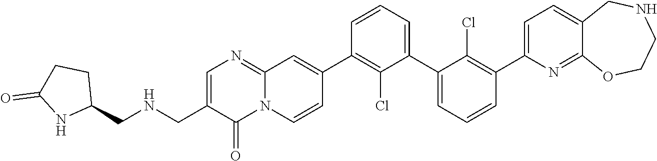 [SMILES: O=C1CC[C@@H](CNCc2cnc3cc(-c4cccc(-c5cccc(-c6ccc7c(n6)OCCNC7)c5Cl)c4Cl)ccn3c2=O)N1]

### tert-Butyl 8-(2,2′-dichloro-3′-(3-formyl-4-oxo-4H-pyrido[1,2-a]pyrimidin-8-yl)-[1,1′-biphenyl]-3-yl)-2,3-dihydropyrido[3,2-f][1,4]oxazepine-4(5H)-carboxylate

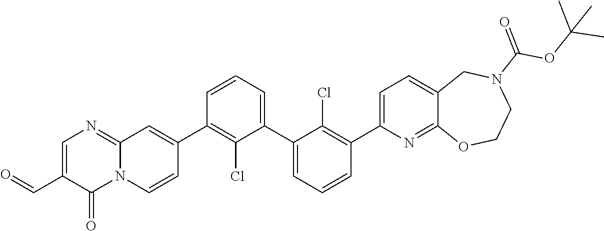 [SMILES: CC(C)(C)OC(=O)N1CCOc2nc(-c3cccc(-c4cccc(-c5ccn6c(=O)c(C=O)cnc6c5)c4Cl)c3Cl)ccc2C1]

Pd(PPh₃)₄ (67 mg, 0.06 mmol), potassium carbonate, (120 mg, 0.86 mmol), 8-[2-chloro-3-[2-chloro-3-(4,4,5,5-tetramethyl-1,3,2-dioxaborolan-2-yl)phenyl]phenyl]-4-oxo-pyrido[1,2-a]pyrimidine-3-carbaldehyde (150 mg, 0.29 mmol), and tert-butyl 8-chloro-3,5-dihydro-2H-pyrido[3,2-f][1,4]oxazepine-4-carboxylate (115 mg, 0.40 mmol) were suspended in dioxane/water (4:1, 5 mL), then the solution was heated at 105° C. for 20 minutes. Reaction was cooled to rt, diluted with 5 mL water, and extracted with EtOAc (3×3 mL). The combined organics were concentrated under reduced pressure and crude sample was purified by normal phase SiO₂ chromatography (5-70_{%} EtOAc/hexane) to afford tert-butyl 8-[2-chloro-3-[2-chloro-3-(3-formyl-4-oxo-pyrido[1,2-a]pyrimidin-8-yl)phenyl]phenyl]-3, 5-dihydro-2H-pyrido[3,2-f][1,4]oxazepine-4-carboxylate (96 mg, 51.8% yield) as a yellow foam. MS: m/z found 643.3, 645.3 [M+H]⁺.

### 8-(2,2′-Dichloro-3′-(2,3,4,5-tetrahydropyrido[3,2-f][1,4]oxazepin-8-yl)-[1,1′-biphenyl]-3-yl)-4-oxo-4H-pyrido[1,2-a]pyrimidine-3-carbaldehyde

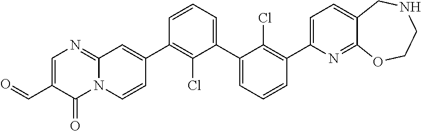 [SMILES: O=Cc1cnc2cc(-c3cccc(-c4cccc(-c5ccc6c(n5)OCCNC6)c4Cl)c3Cl)ccn2c1=O]

tert-butyl 8-[2-chloro-3-[2-chloro-3-(3-formyl-4-oxo-pyrido[1,2-a]pyrimidin-8-yl)phenyl]phenyl]-3,5-dihydro-2H-pyrido[3,2-f][1,4]oxazepine-4-carboxylate (96 mg, 0.15 mmol) was dissolved in 2 mL DCM and 4M HCl solution in dioxane (112 μL, 0.45 mmol) was added. Reaction was stirred at rt for 60 minutes and solvent was removed under reduced pressure. Crude residue was dissolved in 3 mL water and the pH was adjusted to −10 by slow addition of saturated aqueous sodium bicarbonate solution. The resulting aqueous was extracted with DCM (3×3 mL) and the combined organics were dried over sodium sulfate, filtered, and concentrated under reduced pressure to afford 8-[2-chloro-3-[2-chloro-3-(2,3,4,5-tetrahydropyrido[3,2-f][1,4]oxazepin-8-yl)phenyl]phenyl]-4-oxo-pyrido[1,2-a]pyrimidine-3-carbaldehyde (81 mg, crude) as a yellow oil. MS: m/z found 543.2, 545.2 [M+H]⁺. Material was used for the next step without further purification.

### (S)-8-(2,2′-Dichloro-3′-(2,3,4,5-tetrahydropyrido[3,2-f][1,4]oxazepin-8-yl)-[1,1′-biphenyl]-3-yl)-3-((((5-oxopyrrolidin-2-yl)methyl)amino)methyl)-4H-pyrido[1,2-a]pyrimidin-4-one

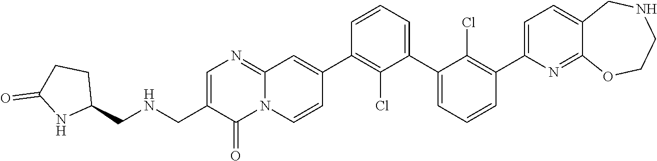 [SMILES: O=C1CC[C@@H](CNCc2cnc3cc(-c4cccc(-c5cccc(-c6ccc7c(n6)OCCNC7)c5Cl)c4Cl)ccn3c2=O)N1]

8-[2-Chloro-3-[2-chloro-3-(2,3,4,5-tetrahydropyrido[3,2-f][1,4]oxazepin-8-yl)phenyl]phenyl]-4-oxo-pyrido[1,2-a]pyrimidine-3-carbaldehyde (40 mg, 0.07 mmol), (5S)-5-(aminomethyl)pyrrolidin-2-one (21 mg, 0.18 mmol), and acetic acid (7 mg, 0.11 mmol) were dissolved in MeOH/THF (1:1, 1 mL), then the solution was stirred at 25° C. for 2 hours. NaBH₃CN (9 mg, 0.15 mmol) was added and the resulting mixture was stirred at 25° C. for 1 hour. Reaction was subsequently diluted with water (2 mL) and extracted with EtOAc (3×2 mL). The combined organics were further washed with brine, dried over sodium sulfate, filtered, and concentrated under reduced pressure. Crude sample was purified by reverse phase HPLC to afford 8-[2-chloro-3-[2-chloro-3-(2,3,4,5-tetrahydropyrido[3,2-f1][1,4]oxazepin-8-yl)phenyl]phenyl]-3-[[[(2S)-5-oxopyrrolidin-2-yl]methylamino]methyl]pyrido[1,2-a]pyrimidin-4-one (13.3 mg, 28.2% yield) as a white foam. MS: m/z found 641, 643 [M+H]⁺. ¹H NMR formic acid salt (400 MHz, Methanol-d₄) δ 9.19-9.11 (m, 1H), 8.44 (d, J=11.6 Hz, 2H), 7.89 (d, J=7.6 Hz, 1H), 7.81 (dd, J=1.9, 0.7 Hz, 1H), 7.66-7.48 (m, 6H), 7.47-7.39 (m, 2H), 4.37-4.29 (m, 2H), 4.19 (s, 2H), 4.11-4.01 (m, 2H), 3.94 (s, 1H), 3.44-3.37 (m, 2H), 2.95 (qd, J=12.5, 6.2 Hz, 2H), 2.42-2.27 (m, 3H), 1.92-1.79 (m, 1H).

### Example 267: (S)-5-((((6-(2,2′-Dichloro-3′-(4-(2-hydroxyethyl)-2,3,4,5-tetrahydropyrido[3,2-f][1,4]oxazepin-8-yl)-[1,1′-biphenyl]-3-yl)-2-methoxypyridin-3-yl)methyl)amino)methyl)pyrrolidin-2-one

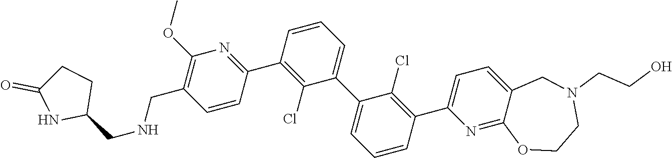 [SMILES: COc1nc(-c2cccc(-c3cccc(-c4ccc5c(n4)OCCN(CCO)C5)c3Cl)c2Cl)ccc1CNC[C@@H]1CCC(=O)N1]

### 2-(8-Chloro-2,3-dihydropyrido[3,2-f][1,4]oxazepin-4(5H)-yl)ethan-1-ol

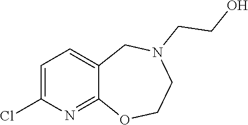 [SMILES: OCCN1CCOc2nc(Cl)ccc2C1]

8-chloro-2,3,4,5-tetrahydropyrido[3,2-f][1,4]oxazepine; hydrochloride (70 mg, 0.32 mmol), DIEA (220 μL, 163 mg, 1.27 mmol), and 2-bromoethanol (117 μL, 198 mg, 1.58 mmol) were dissolved in 2 mL THF, then the solution was heated at 75° C. for 12 hours. Reaction was diluted with 5 mL water and extracted with EtOAc (3×3 mL). The combined organics were further washed with brine (3 mL), dried over sodium sulfate, filtered, and concentrated under reduced pressure to afford 2-(8-chloro-3,5-dihydro-2H-pyrido[3,2-f][1,4]oxazepin-4-yl)ethanol (68.8 mg, crude) as a yellow oil. MS: m/z found 229.1 [M+H]⁺. Material was used for the next step without further purification.

### tert-Butyl (S)-((6-(2,2′-dichloro-3′-(4-(2-hydroxyethyl)-2,3,4,5-tetrahydropyrido[3,2-f][1,4]oxazepin-8-yl)-[1,1′-biphenyl]-3-yl)-2-methoxypyridin-3-yl)methyl)((5-oxopyrrolidin-2-yl)methyl)carbamate

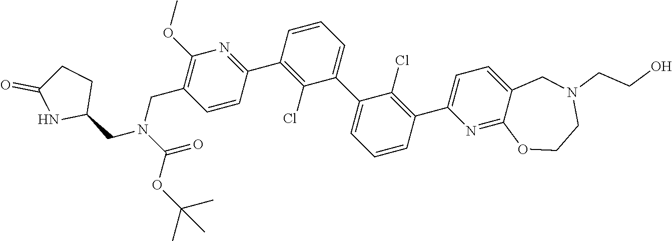 [SMILES: COc1nc(-c2cccc(-c3cccc(-c4ccc5c(n4)OCCN(CCO)C5)c3Cl)c2Cl)ccc1CN(C[C@@H]1CCC(=O)N1)C(=O)OC(C)(C)C]

Pd(PPh₃)₄ (17 mg, 0.01 mmol), potassium carbonate (30 mg, 0.22 mmol), tert-butyl N-[[6-[2-chloro-3-[2-chloro-3-(4,4,5,5-tetramethyl-1,3,2-dioxaborolan-2-yl)phenyl]phenyl]-2-methoxy-3-pyridyl]methyl]-N-[[(2S)-5-oxopyrrolidin-2-yl]methyl]carbamate (50 mg, 0.07 mmol), and 2-(8-chloro-3,5-dihydro-2H-pyrido[3,2-f][1,4]oxazepin-4-yl)ethanol (25 mg, 0.11 mmol) were suspended in dioxane/water (4:1 (v v), 1 mL), then the solution was heated at 105° C. for 20 minutes. Reaction was cooled to rt, diluted with 3 mL water, and extracted with EtOAc (3×2 mL). The combined organics were concentrated under reduced pressure and crude sample was purified by reverse phase HPLC to afford tert-butyl N-[[6-[2-chloro-3-[2-chloro-3-[4-(2-hydroxyethyl)-3, 5-dihydro-2H-pyrido[3,2-f][1,4]oxazepin-8-yl]phenyl]phenyl]-2-methoxy-3-pyridyl]methyl]-N-[[(2S)-5-oxopyrrolidin-2-yl]methyl]carbamate (6.4 mg, 11.7% yield) as a clear film. MS: m/z found 748.45, 750 [M+H]⁺.

### (S)-5-((((6-(2,2′-Dichloro-3′-(4-(2-hydroxyethyl)-2,3,4,5-tetrahydropyrido[3,2-f][1,4]oxazepin-8-yl)-[1,1′-biphenyl]-3-yl)-2-methoxypyridin-3-yl)methyl)amino)methyl)pyrrolidin-2-one

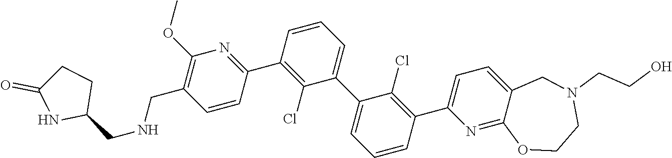 [SMILES: COc1nc(-c2cccc(-c3cccc(-c4ccc5c(n4)OCCN(CCO)C5)c3Cl)c2Cl)ccc1CNC[C@@H]1CCC(=O)N1]

tert-Butyl N-[[6-[2-chloro-3-[2-chloro-3-[4-(2-hydroxyethyl)-3,5-dihydro-2H-pyrido[3,2-f1][1,4]oxazepin-8-yl]phenyl]phenyl]-2-methoxy-3-pyridyl]methyl]-N-[[(2S)-5-oxopyrrolidin-2-yl]methyl]carbamate (6.4 mg, 0.01 mmol) was dissolved in 0.5 mL DCM and 4M HCl solution in dioxane (8.6 μL, 0.03 mmol) was added. Reaction was stirred at rt for 20 minutes and solvent was removed under reduced pressure. Crude sample was purified by reverse phase HPLC to afford (5S)-5-[[[6-[2-chloro-3-[2-chloro-3-[4-(2-hydroxyethyl)-3,5-dihydro-2H-pyrido[3,2-f][1,4]oxazepin-8-yl]phenyl]phenyl]-2-methoxy-3-pyridyl]methylamino]methyl]pyrrolidin-2-one (5.10 mg, 92% yield) as a white foam. MS: m/z found 648.45, 650 [M+H]⁺. ¹H NMR (400 MHz, Chloroform-d) δ 7.61 (ddd, J=23.2, 12.7, 7.8 Hz, 4H), 7.46-7.38 (m, 3H), 7.31 (dd, J=15.2, 7.5 Hz, 3H), 4.26 (d, J=5.0 Hz, 2H), 4.02 (s, 3H), 3.91 (s, 2H), 3.78 (d, J=16.1 Hz, 4H), 3.66 (q, J=5.4 Hz, 3H), 3.20 (d, J=4.9 Hz, 2H), 2.82-2.71 (m, 3H), 2.59-2.52 (m, 1H), 2.36 (t, J=8.1 Hz, 2H), 2.24 (dq, J=14.7, 7.5 Hz, 1H), 1.76 (d, J=18.3 Hz, 1H).

### Example 268: (S)-8-(2,2′-Dichloro-3′-(4-(2-hydroxyethyl)-2,3,4,5-tetrahydropyrido[3,2-f][1,4]oxazepin-8-yl)-[1,1′-biphenyl]-3-yl)-3-((((5-oxopyrrolidin-2-yl)methyl)amino)methyl)-4H-pyrido[1,2-a]pyrimidin-4-one

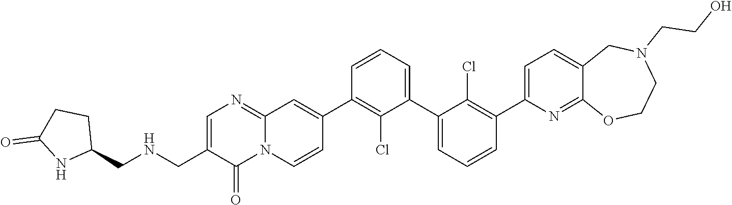 [SMILES: O=C1CC[C@@H](CNCc2cnc3cc(-c4cccc(-c5cccc(-c6ccc7c(n6)OCCN(CCO)C7)c5Cl)c4Cl)ccn3c2=O)N1]

### tert-Butyl (S)-((8-(2,2′-dichloro-3′-(4-(2-hydroxyethyl)-2,3,4,5-tetrahydropyrido[3,2-f][1,4]oxazepin-8-yl)-[1,1′-biphenyl]-3-yl)-4-oxo-4H-pyrido[1,2-a]pyrimidin-3-yl)methyl)((5-oxopyrrolidin-2-yl)methyl)carbamate

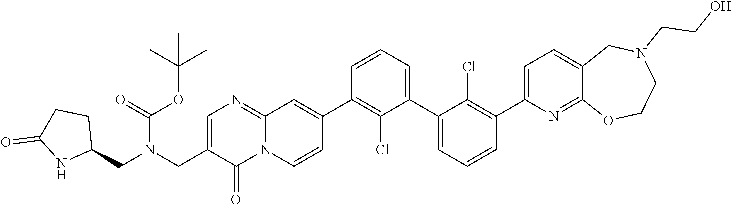 [SMILES: CC(C)(C)OC(=O)N(Cc1cnc2cc(-c3cccc(-c4cccc(-c5ccc6c(n5)OCCN(CCO)C6)c4Cl)c3Cl)ccn2c1=O)C[C@@H]1CCC(=O)N1]

Pd(PPh₃)₄ (15 mg, 0.01 mmol), potassium carbonate (28 mg, 0.20 mmol), tert-butyl N-[[8-[2-chloro-3-[2-chloro-3-(4,4,5,5-tetramethyl-1,3,2-dioxaborolan-2-yl)phenyl]phenyl]-4-oxo-pyrido[1,2-a]pyrimidin-3-yl]methyl]-N-[[(2S)-5-oxopyrrolidin-2-yl]methyl]carbamate (60 mg, 0.07 mmol), and 2-(8-chloro-3,5-dihydro-2H-pyrido[3,2-f][1,4]oxazepin-4-yl)ethanol (20 mg, 0.09 mmol) were suspended in dioxane/water (4:1, 1 mL), then the solution was heated at 105° C. for 20 minutes. Reaction was cooled to rt, diluted with 3 mL water, and extracted with EtOAc (3×2 mL). The combined organics were concentrated under reduced pressure and crude sample was purified by reverse phase HPLC to afford tert-butyl N-[[8-[2-chloro-3-[2-chloro-3-[4-(2-hydroxyethyl)-3,5-dihydro-2H-pyrido[3,2-f][1,4]oxazepin-8-yl]phenyl]phenyl]-4-oxo-pyrido[1,2-a]pyrimidin-3-yl]methyl]-N-[[(2S)-5-oxopyrrolidin-2-yl]methyl]carbamate (5.5 mg, 10.5% yield) as a clear oil. MS: m/z found 785.4, 787.4 [M+H]⁺.

### (S)-8-(2,2′-Dichloro-3′-(4-(2-hydroxyethyl)-2,3,4,5-tetrahydropyrido[3,2-f][1,4]oxazepin-8-yl)-[1,1′-biphenyl]-3-yl)-3-((((5-oxopyrrolidin-2-yl)methyl)amino)methyl)-4H-pyrido[1,2-a]pyrimidin-4-one

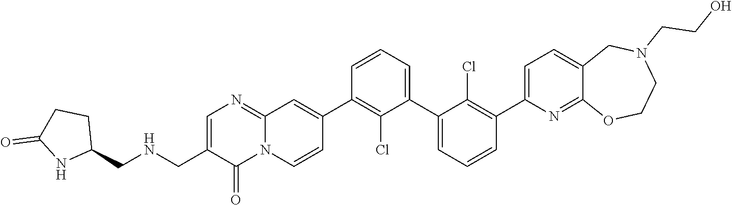 [SMILES: O=C1CC[C@@H](CNCc2cnc3cc(-c4cccc(-c5cccc(-c6ccc7c(n6)OCCN(CCO)C7)c5Cl)c4Cl)ccn3c2=O)N1]

tert-Butyl N-[[8-[2-chloro-3-[2-chloro-3-[4-(2-hydroxyethyl)-3,5-dihydro-2H-pyrido[3,2-f][1,4]oxazepin-8-yl]phenyl]phenyl]-4-oxo-pyrido[1,2-a]pyrimidin-3-yl]methyl]-N-[[(2S)-5-oxopyrrolidin-2-yl]methyl]carbamate (5.5 mg, 0.01 mmol) was dissolved in 0.5 mL DCM and 4 M HCl solution in dioxane (7 μL, 0.03 mmol) was added. Reaction was stirred at rt for 20 minutes and solvent was removed under reduced pressure. Crude sample was purified by reverse phase HPLC to afford 8-[2-chloro-3-[2-chloro-3-[4-(2-hydroxyethyl)-3,5-dihydro-2H-pyrido[3,2-f][1,4]oxazepin-8-yl]phenyl]phenyl]-3-[[[(2S)-5-oxopyrrolidin-2-yl]methylamino]methyl]pyrido[1,2-a]pyrimidin-4-one (4.3 mg, 89.6% yield) as a white foam. MS: m/z found 685, 687 [M+H]⁺. ¹H NMR (400 MHz, Chloroform-d) δ 9.08 (d, J=7.4 Hz, 1H), 8.33 (s, 1H), 7.76 (d, J=1.9 Hz, 1H), 7.69-7.57 (m, 2H), 7.53-7.41 (m, 5H), 7.38 (dd, J=7.5, 2.0 Hz, 1H), 7.33 (dd, J=7.7, 1.7 Hz, 1H), 6.14 (s, 1H), 4.34-4.17 (m, 2H), 3.92 (s, 2H), 3.86 (d, J=5.0 Hz, 2H), 3.78 (s, 1H), 3.67 (q, J=7.9, 6.6 Hz, 2H), 3.20 (s, 2H), 2.85-2.73 (m, 3H), 2.56 (dd, J=11.9, 8.8 Hz, 1H), 2.35 (t, J=8.1 Hz, 2H), 2.29-2.18 (m, 1H), 1.77 (s, 1H), 0.85 (d, J=16.8 Hz, 2H).

### Example 281: (S)-8-(2,2′-Dichloro-3′-(6-methoxy-5-((((5-oxopyrrolidin-2-yl)methyl)-amino)-methyl)pyridin-2-yl)-[1,1′-biphenyl]-3-yl)-3-(((2-hydroxyethyl)(methyl)amino)methyl)-4H-pyrido[1,2-a]-pyrimidin-4-one

Compound was prepared in the same manner as described for Example 80, utilizing intermediate (S)-tert-butyl ((6-(2,2′-dichloro-3′-(3-formyl-4-oxo-4H-pyrido[1,2-a]pyrimidin-8-yl)-[1,1′-biphenyl]-3-yl)-2-methoxypyridin-3-yl)methyl)((5-oxopyrrolidin-2-yl)methyl)carbamate. 2-aminoethan-1-ol was replaced by 2-(methylamino)-ethan-1-ol.

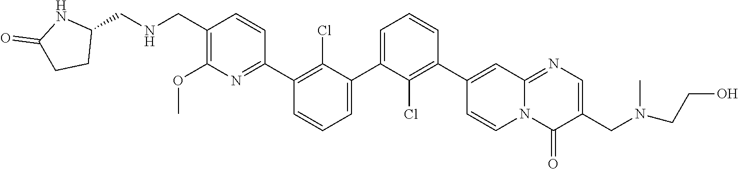 [SMILES: COc1nc(-c2cccc(-c3cccc(-c4ccn5c(=O)c(CN(C)CCO)cnc5c4)c3Cl)c2Cl)ccc1CNC[C@@H]1CCC(=O)N1]

White solid. MS: m/z found 687.3, 689 [M+H]⁺. ¹H NMR formic acid salt (400 MHz, Methanol-d₄) δ 9.18 (d, J=7.4 Hz, 1H), 8.51 (s, 2H), 7.84 (s, 1H), 7.76 (d, J=7.5 Hz, 1H), 7.68-7.58 (m, 4H), 7.51 (t, J=7.4 Hz, 2H), 7.40 (d, J=7.7 Hz, 1H), 7.26 (d, J=7.4 Hz, 1H), 4.19 (s, 2H), 4.02 (s, 3H), 3.93-3.83 (m, 5H), 3.12 (s, 2H), 2.75 (d, J=21.6 Hz, 5H), 2.41-2.26 (m, 3H), 1.83 (s, 1H).

### Example 282: (S)-5-((((6-(2,2′-Dichloro-3′-(3-(((((S)-5-oxopyrrolidin-2-yl)-methyl)amino)-methyl)-pyrazolo[1,5-a]pyrimidin-5-yl)-[1,1′-biphenyl]-3-yl)-2-methoxypyridin-3-yl)methyl)-amino)methyl)-pyrrolidin-2-one

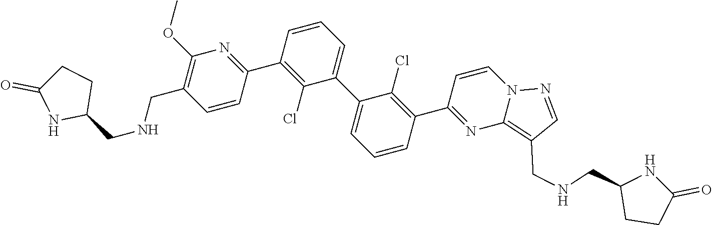 [SMILES: COc1nc(-c2cccc(-c3cccc(-c4ccn5ncc(CNC[C@@H]6CCC(=O)N6)c5n4)c3Cl)c2Cl)ccc1CNC[C@@H]1CCC(=O)N1]

### tert-Butyl (S)-((6-(2,2′-dichloro-3′-(3-formylpyrazolo[1,5-a]pyrimidin-5-yl)-[1,1′-biphenyl]-3-yl)-2-methoxypyridin-3-yl)methyl)((5-oxopyrrolidin-2-yl)methyl)carbamate

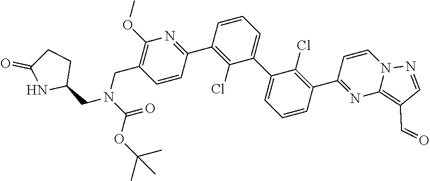 [SMILES: COc1nc(-c2cccc(-c3cccc(-c4ccn5ncc(C=O)c5n4)c3Cl)c2Cl)ccc1CN(C[C@@H]1CCC(=O)N1)C(=O)OC(C)(C)C]

Pd(PPh₃)₄ (20.31 mg, 0.02 mmol), potassium carbonate (36.4 mg, 0.26 mmol), tert-butyl N-[[6-[2-chloro-3-[2-chloro-3-(4,4,5,5-tetramethyl-1,3,2-dioxaborolan-2-yl)phenyl]phenyl]-2-methoxy-3-pyridyl]methyl]-N-[[(2S)-5-oxopyrrolidin-2-yl]methyl]carbamate (60. mg, 0.09 mmol), and 5-chloropyrazolo[1,5-a]pyrimidine-3-carbaldehyde (24 mg, 0.13 mmol) were suspended in dioxane/water (4:1, 2 mL), then the solution was heated at 105° C. for 20 minutes. Reaction was cooled to rt, diluted with 3 mL water, and extracted with EtOAc (3×2 mL). The combined organics were concentrated under reduced pressure and crude sample was purified by normal phase SiO₂ chromatography (10-100% EtOAc/hexane) to afford tert-butyl N-[[6-[2-chloro-3-[2-chloro-3-(3-formylpyrazolo[1,5-a]pyrimidin-5-yl)phenyl]phenyl]-2-methoxy-3-pyridyl]methyl]-N-[[(2S)-5-oxopyrrolidin-2-yl]methyl]carbamate (46 mg, 74.4% yield) as a yellow foam. MS: m/z found 701.3, 703[M+H]⁺.

### tert-Butyl ((6-(2,2′-dichloro-3′-(3-(((((S)-5-oxopyrrolidin-2-yl)methyl)amino)methyl)pyrazolo[1,5-a]pyrimidin-5-yl)-[1,1′-biphenyl]-3-yl)-2-methoxypyridin-3-yl)methyl)(((S)-5-oxopyrrolidin-2-yl)methyl)carbamate

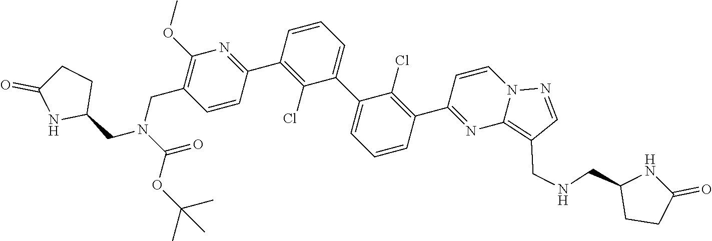 [SMILES: COc1nc(-c2cccc(-c3cccc(-c4ccn5ncc(CNC[C@@H]6CCC(=O)N6)c5n4)c3Cl)c2Cl)ccc1CN(C[C@@H]1CCC(=O)N1)C(=O)OC(C)(C)C]

tert-Butyl N-[[6-[2-chloro-3-[2-chloro-3-(3-formylpyrazolo[1,5-a]pyrimidin-5-yl)phenyl]phenyl]-2-methoxy-3-pyridyl]methyl]-N-[[(2S)-5-oxopyrrolidin-2-yl]methyl]carbamate (46 mg, 0.07 mmol), (5S)-5-(aminomethyl)pyrrolidin-2-one (10 mg, 0.09 mmol), and acetic acid (6 mg, 0.10 mmol) were dissolved in MeOH/THF (1:1, 2 mL), then the solution was stirred at 25° C. for 2 hours. NaBH₃CN (8 mg, 0.13 mmol) was then added and the resulting mixture was stirred at 25° C. for 1 hour. Reaction was subsequently diluted with water (2 mL) and extracted with DCM (3×2 mL). The combined organics were further washed with brine (10 mL), dried over sodium sulfate, filtered, and concentrated under reduced pressure to afford tert-butyl N-[[6-[2-chloro-3-[2-chloro-3-[3-[[[(2S)-5-oxopyrrolidin-2-yl]methylamino]methyl]pyrazolo[1,5-a]pyrimidin-5-yl]phenyl]phenyl]-2-methoxy-3-pyridyl]methyl]-N-[[(2S)-5-oxopyrrolidin-2-yl]methyl]carbamate (52 mg, crude) as a white foam. MS: m/z found 799.4, 801 [M+H]⁺. Material was used for the next step without further purification.

### (S)-5-((((6-(2,2′-Dichloro-3′-(3-(((((S)-5-oxopyrrolidin-2-yl)methyl)amino)methyl)pyrazolo[1,5-a]pyrimidin-5-yl)-[1,1′-biphenyl]-3-yl)-2-methoxypyridin-3-yl)methyl)amino)methyl)pyrrolidin-2-one

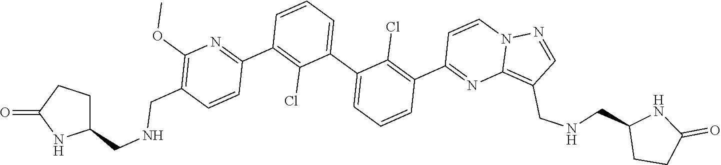 [SMILES: COc1nc(-c2cccc(-c3cccc(-c4ccn5ncc(CNC[C@@H]6CCC(=O)N6)c5n4)c3Cl)c2Cl)ccc1CNC[C@@H]1CCC(=O)N1]

tert-Butyl N-[[6-[2-chloro-3-[2-chloro-3-[3-[[[(2S)-5-oxopyrrolidin-2-yl]methylamino]methyl]pyrazolo[1,5-a]pyrimidin-5-yl]phenyl]phenyl]-2-methoxy-3-pyridyl]methyl]-N-[[(2S)-5-oxopyrrolidin-2-yl]methyl]carbamate (52 mg, 0.07 mmol) was dissolved in 1 mL DCM and 4M HCl solution in dioxane (65 μL, 0.26 mmol) was added. Reaction was stirred at rt for 20 minutes and solvent was removed under reduced pressure. Crude sample was purified by reverse phase HPLC to afford (5S)-5-[[[6-[2-chloro-3-[2-chloro-3-[3-[[[(2S)-5-oxopyrrolidin-2-yl]-methylamino]-methyl]-pyrazolo[1,5-a]-pyrimidin-5-yl]phenyl]phenyl]-2-methoxy-3-pyridyl]methylamino]methyl]pyrrolidin-2-one (18 mg, 39.8% yield) as a white foam. MS: m/z found 699.4, 671 [M+H]⁺. ¹H NMR formic acid salt (400 MHz, Methanol-d₄) δ 9.02 (d, J=7.3 Hz, 1H), 8.47 (s, 1H), 8.33 (s, 1H), 7.81-7.72 (m, 2H), 7.66 (dd, J=8.0, 1.7 Hz, 1H), 7.60 (t, J=7.7 Hz, 1H), 7.52 (dq, J=7.6, 4.5, 3.2 Hz, 2H), 7.39 (t, J=8.0 Hz, 2H), 7.28 (d, J=7.4 Hz, 1H), 4.37 (s, 2H), 4.04 (s, 3H), 4.00-3.88 (m, 4H), 3.03 (dt, J=12.5, 7.2 Hz, 2H), 2.92-2.81 (m, 2H), 2.34 (dd, J=13.9, 10.8 Hz, 6H), 1.85 (dd, J=12.3, 7.5 Hz, 2H).

### Example 283: (S)-8-(3′-(5-(1-(Aminomethyl)cyclopropyl)-6-methoxypyridin-2-yl)-2,2′-dichloro-[1,1′-biphenyl]-3-yl)-3-((((5-oxopyrrolidin-2-yl)methyl)amino)methyl)-4H-pyrido[1,2-a]pyrimidin-4-one

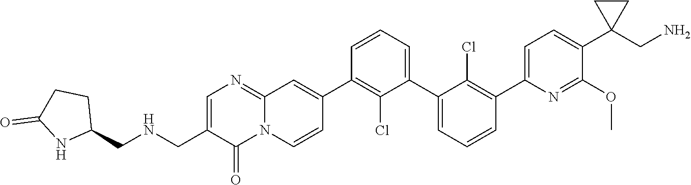 [SMILES: COc1nc(-c2cccc(-c3cccc(-c4ccn5c(=O)c(CNC[C@@H]6CCC(=O)N6)cnc5c4)c3Cl)c2Cl)ccc1C1(CN)CC1]

### tert-Butyl (S)-((8-(3′-(5-(1-(aminomethyl)cyclopropyl)-6-methoxypyridin-2-yl)-2,2′-dichloro-[1,1′-biphenyl]-3-yl)-4-oxo-4H-pyrido[1,2-a]pyrimidin-3-yl)methyl)((5-oxopyrrolidin-2-yl)methyl)carbamate

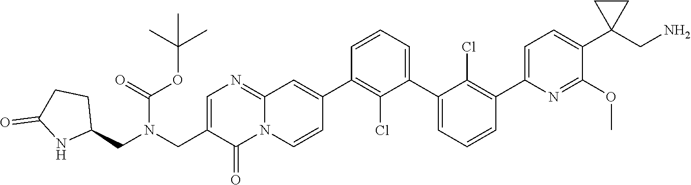 [SMILES: COc1nc(-c2cccc(-c3cccc(-c4ccn5c(=O)c(CN(C[C@@H]6CCC(=O)N6)C(=O)OC(C)(C)C)cnc5c4)c3Cl)c2Cl)ccc1C1(CN)CC1]

Pd(PPh₃)₄ (3.6 mg, 0.001 mmol), potassium carbonate (6.4 mg, 0.05 mmol), tert-butyl N-[[8-[2-chloro-3-[2-chloro-3-(4,4,5,5-tetramethyl-1,3,2-dioxaborolan-2-yl)phenyl]phenyl]-4-oxo-pyrido[1,2-a]pyrimidin-3-yl]methyl]-N-[[(2S)-5-oxopyrrolidin-2-yl]methyl]carbamate (14 mg, 0.02 mmol), and [1-(6-bromo-2-methoxy-3-pyridyl)cyclopropyl]methanamine, (8 mg, 0.03 mmol) were suspended in dioxane/water (4:1, 1 mL), then the solution was heated at 105° C. for 20 minutes. Reaction was cooled to rt, diluted with 2 mL water, and extracted with EtOAc (3×2 mL). The combined organics were concentrated under reduced pressure and crude sample was purified by reverse phase HPLC to afford tert-butyl N-[[8-[3-[3-[5-[1-(aminomethyl)cyclopropyl]-6-methoxy-2-pyridyl]-2-chloro-phenyl]-2-chloro-phenyl]-4-oxo-pyrido[1,2-a]pyrimidin-3-yl]methyl]-N-[[(2S)-5-oxopyrrolidin-2-yl]methyl]carbamate (8 mg, 66.8% yield) as a clear oil. MS: m/z found 769.4, 771.5 [M+H]⁺.

### (S)-8-(3′-(5-(1-(Aminomethyl)cyclopropyl)-6-methoxypyridin-2-yl)-2,2′-dichloro-[1,1′-biphenyl]-3-yl)-3-((((5-oxopyrrolidin-2-yl)methyl)amino)methyl)-4H-pyrido[1,2-a]pyrimidin-4-one

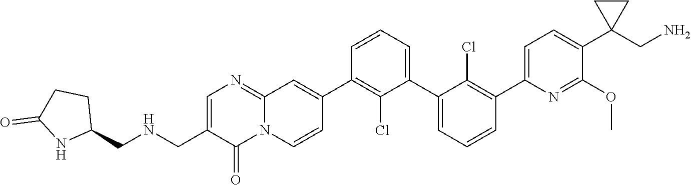 [SMILES: COc1nc(-c2cccc(-c3cccc(-c4ccn5c(=O)c(CNC[C@@H]6CCC(=O)N6)cnc5c4)c3Cl)c2Cl)ccc1C1(CN)CC1]

tert-Butyl N-[[8-[3-[3-[5-[1-(aminomethyl)cyclopropyl]-6-methoxy-2-pyridyl]-2-chloro-phenyl]-2-chloro-phenyl]-4-oxo-pyrido[1,2-a]pyrimidin-3-yl]methyl]-N-[[(2S)-5-oxopyrrolidin-2-yl]methyl]carbamate (8 mg, 0.01 mmol) was dissolved in 0.5 mL DCM and 4M HCl solution in dioxane (10 μL, 1.5 mg, 0.04 mmol) was added. Reaction was stirred at rt for 20 minutes and solvent was removed under reduced pressure. Crude sample was purified by reverse phase HPLC to afford 8-[3-[3-[5-[1-(aminomethyl)cyclopropyl]-6-methoxy-2-pyridyl]-2-chloro-phenyl]-2-chloro-phenyl]-3-[[[(2S)-5-oxopyrrolidin-2-yl]methylamino]methyl]-pyrido[1,2-a]pyrimidin-4-one (6.1 mg, 87.7% yield) as white foam. MS: m/z found 669. 671 [M+H]⁺. ¹H NMR formic acid salt (400 MHz, Methanol-d₄) δ 9.12 (d, J=7.4 Hz, 1H), 8.40 (s, 1H), 7.76 (s, 1H), 7.71-7.56 (m, 4H), 7.50 (q, J=8.5, 7.1 Hz, 3H), 7.37 (d, J=7.2 Hz, 1H), 7.21 (d, J=7.5 Hz, 1H), 4.00 (s, 3H), 3.91-3.79 (m, 3H), 2.82-2.62 (m, 4H), 2.29 (td, J=14.8, 13.1, 6.4 Hz, 3H), 1.81 (d, J=14.0 Hz, 1H), 0.88-0.72 (m, 4H).

### Example 284: 8-(2,2′-Dichloro-3′-(6-methoxy-5-(((((S)-5-oxopyrrolidin-2-yl)methyl)-amino)methyl)-pyridin-2-yl)-[1,1′-biphenyl]-3-yl)-3-(((((S)-5-oxopyrrolidin-3-yl)methyl)amino)methyl)-4H-pyrido[1,2-a]pyrimidin-4-one

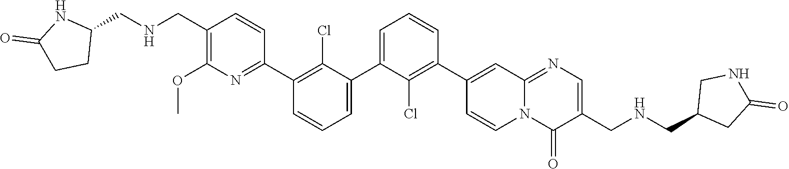 [SMILES: COc1nc(-c2cccc(-c3cccc(-c4ccn5c(=O)c(CNC[C@H]6CNC(=O)C6)cnc5c4)c3Cl)c2Cl)ccc1CNC[C@@H]1CCC(=O)N1]

Compound was prepared in the same manner as described for Example 80, utilizing intermediate (S)-tert-butyl ((6-(2,2′-dichloro-3′-(3-formyl-4-oxo-4H-pyrido[1,2-a]pyrimidin-8-yl)-[1,1′-biphenyl]-3-yl)-2-methoxypyridin-3-yl)methyl)((5-oxopyrrolidin-2-yl)methyl)carbamate. 2-aminoethan-1-ol was replaced by (S)-4-(aminomethyl)pyrrolidin-2-one. White foam. MS: m/z found 726.4, 728 [M+H]⁺. ¹H NMR formic acid salt (400 MHz, Methanol-d₄) δ 9.20-9.12 (m, 1H), 8.48 (s, 2H), 7.85-7.81 (m, 1H), 7.77 (d, J=7.5 Hz, 1H), 7.68-7.57 (m, 4H), 7.55-7.49 (m, 2H), 7.40 (dd, J=7.6, 1.8 Hz, 1H), 7.27 (d, J=7.4 Hz, 1H), 4.15 (s, 2H), 4.03 (s, 3H), 3.92 (dd, J=18.7, 4.9 Hz, 3H), 3.58 (dd, J=10.1, 8.0 Hz, 1H), 3.17 (dd, J=10.1, 6.3 Hz, 1H), 3.06 (d, J=7.7 Hz, 2H), 2.90-2.76 (m, 3H), 2.53 (dd, J=16.9, 8.8 Hz, 1H), 2.40-2.26 (m, 3H), 2.19 (dd, J=16.9, 7.5 Hz, 1H), 1.91-1.78 (m, 1H).

### Example 316: (S)-8-(2,2′-Dichloro-3′-(6-methoxy-5-((((5-oxopyrrolidin-2-yl)methyl)-amino)methyl)-pyridin-2-yl)-[1,1′-biphenyl]-3-yl)-3-(((3-hydroxypropyl)amino)methyl)-4H-pyrido[1,2-a]pyrimidin-4-one

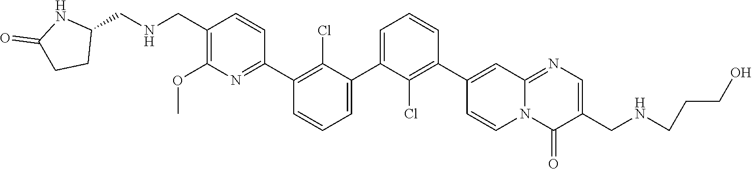 [SMILES: COc1nc(-c2cccc(-c3cccc(-c4ccn5c(=O)c(CNCCCO)cnc5c4)c3Cl)c2Cl)ccc1CNC[C@@H]1CCC(=O)N1]

Compound was prepared in the same manner as described for Example 80, utilizing intermediate (S)-tert-butyl ((6-(2,2′-dichloro-3′-(3-formyl-4-oxo-4H-pyrido[1,2-a]pyrimidin-8-yl)-[1,1′-biphenyl]-3-yl)-2-methoxypyridin-3-yl)methyl)((5-oxopyrrolidin-2-yl)methyl)carbamate. 2-aminoethan-1-ol was replaced by 3-aminopropan-1-ol.

White foam. MS: m/z found 687.4, 689.4 [M+H]⁺. ¹H NMR formic acid salt (400 MHz, Methanol-d₄) δ 9.18 (d, J=7.4 Hz, 1H), 8.51 (s, 3H), 7.86 (s, 1H), 7.79 (d, J=7.5 Hz, 1H), 7.64 (d, J=9.3 Hz, 4H), 7.53 (d, J=7.2 Hz, 2H), 7.41 (d, J=7.6 Hz, 1H), 7.28 (d, J=7.4 Hz, 1H), 4.28 (s, 2H), 4.01 (d, J=19.8 Hz, 5H), 3.91 (s, 1H), 3.72 (d, J=6.6 Hz, 2H), 3.24 (t, J=7.2 Hz, 2H), 2.87 (s, 2H), 2.34 (s, 3H), 1.94 (s, 2H), 1.85 (s, 1H).

### Example 317: (S)-8-(2,2′-Dichloro-3′-(6-methoxy-5-((((5-oxopyrrolidin-2-yl)methyl)amino)methyl)pyridin-2-yl)-[1,1′-biphenyl]-3-yl)-3-(((3-hydroxypropyl)(methyl)amino)methyl)-4H-pyrido[1,2-a]pyrimidin-4-one

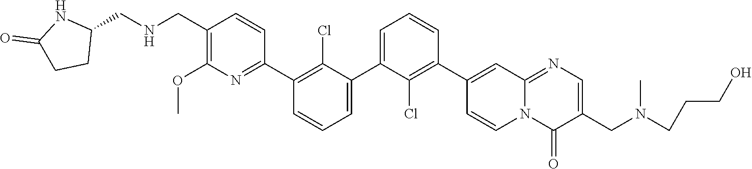 [SMILES: COc1nc(-c2cccc(-c3cccc(-c4ccn5c(=O)c(CN(C)CCCO)cnc5c4)c3Cl)c2Cl)ccc1CNC[C@@H]1CCC(=O)N1]

Compound was prepared in the same manner as described for Example 80, utilizing intermediate (S)-tert-butyl ((6-(2,2′-dichloro-3′-(3-formyl-4-oxo-4H-pyrido[1,2-a]pyrimidin-8-yl)-[1,1′-biphenyl]-3-yl)-2-methoxypyridin-3-yl)methyl)((5-oxopyrrolidin-2-yl)methyl)carbamate. 2-aminoethan-1-ol was replaced by 3-(methylamino)-propan-1-ol. White foam. MS: m/z found 701.3, 703.3 [M+H]⁺. ¹H NMR formic acid salt (400 MHz, Methanol-d₄) δ 9.16 (d, J=7.4 Hz, 1H), 8.53 (s, 1H), 8.47 (s, 1H), 7.82 (s, 1H), 7.74 (d, J=7.4 Hz, 1H), 7.62 (dt, J=15.6, 7.6 Hz, 4H), 7.51 (t, J=7.5 Hz, 2H), 7.40 (d, J=7.8 Hz, 1H), 7.25 (d, J=7.5 Hz, 1H), 4.02 (s, 5H), 3.85 (d, J=4.1 Hz, 3H), 3.68 (t, J=5.9 Hz, 2H), 2.99 (s, 2H), 2.77-2.66 (m, 2H), 2.61 (s, 3H), 2.30 (td, J=14.4, 12.6, 6.2 Hz, 3H), 1.93 (t, J=6.9 Hz, 2H), 1.81 (d, J=11.4 Hz, 1H).

### Example 318: 8-(2,2′-Dichloro-3′-(6-methoxy-5-(((((S)-5-oxopyrrolidin-2-yl)methyl)-amino)-methyl)-pyridin-2-yl)-[1,1′-biphenyl]-3-yl)-3-((((1r,3r)-3-hydroxy-3-methylcyclobutyl)(methyl)amino)methyl)-4H-pyrido[1,2-a]pyrimidin-4-one

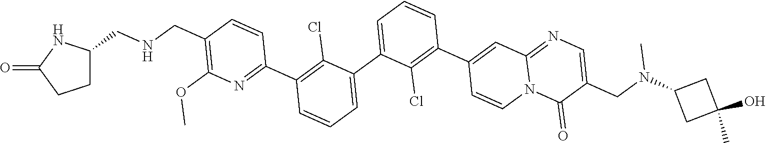 [SMILES: COc1nc(-c2cccc(-c3cccc(-c4ccn5c(=O)c(CN(C)[C@H]6C[C@](C)(O)C6)cnc5c4)c3Cl)c2Cl)ccc1CNC[C@@H]1CCC(=O)N1]

Compound was prepared in the same manner as described for Example 80, utilizing intermediate (S)-tert-butyl ((6-(2,2′-dichloro-3′-(3-formyl-4-oxo-4H-pyrido[1,2-a]pyrimidin-8-yl)-[1,1′-biphenyl]-3-yl)-2-methoxypyridin-3-yl)methyl)((5-oxopyrrolidin-2-yl)methyl)carbamate. 2-aminoethan-1-ol was replaced by (1R,3R)-1-methyl-3-(methylamino)cyclobutan-1-ol (methylamino)-propan-1-ol. White solid. MS: m/z found 727.4, 729.4 [M+H]⁺. ¹H NMR formic acid salt (400 MHz, Chloroform-d) δ 9.09 (dd, J=7.4, 0.7 Hz, 1H), 8.36 (s, 1H), 7.75 (dd, J=1.9, 0.7 Hz, 1H), 7.69 (dd, J=7.7, 1.8 Hz, 1H), 7.59 (d, J=7.5 Hz, 1H), 7.52-7.40 (m, 4H), 7.34 (ddd, J=7.6, 6.6, 1.8 Hz, 2H), 7.27 (d, J=6.8 Hz, 1H), 5.96 (s, 1H), 4.03 (s, 3H), 3.81 (s, 3H), 3.48 (s, 2H), 3.16 (p, J=7.4 Hz, 1H), 2.80 (dd, J=12.0, 4.1 Hz, 1H), 2.60-2.53 (m, 2H), 2.37 (dd, J=8.8, 7.0 Hz, 2H), 2.31-2.19 (m, 4H), 2.14 (s, 3H), 2.11-2.04 (m, 2H), 1.83-1.72 (m, 1H), 1.42 (d, J=1.3 Hz, 3H).

### Example 326: (5S)-5-((((6-(2,2′-Dichloro-3′-(4-((((S)-5-oxopyrrolidin-2-yl)methyl)amino)chroman-7-yl)-[1,1′-biphenyl]-3-yl)-2-methoxypyridin-3-yl)methyl)amino)methyl)pyrrolidin-2-one

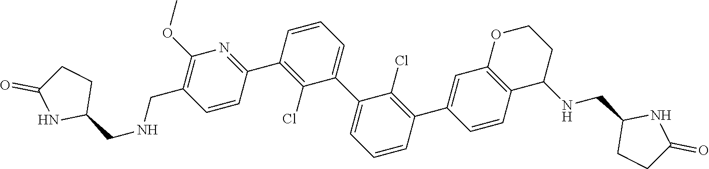 [SMILES: COc1nc(-c2cccc(-c3cccc(-c4ccc5c(c4)OCCC5NC[C@@H]4CCC(=O)N4)c3Cl)c2Cl)ccc1CNC[C@@H]1CCC(=O)N1]

### (5S)-5-(((7-Bromochroman-4-yl)amino)methyl)pyrrolidin-2-one

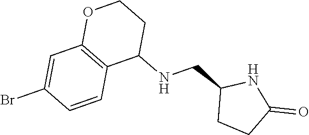 [SMILES: O=C1CC[C@@H](CNC2CCOc3cc(Br)ccc32)N1]

7-bromochroman-4-one (150 mg, 0.66 mmol), (5S)-5-(aminomethyl)-pyrrolidin-2-one (113.11 mg, 0.99 mmol), and tetraethoxytitanium (452 mg, 1.98 mmol) were suspended in 4 mL anhydrous THF. Reaction mixture was heated at 105° C. for 3 hours in a sealed tube. Reaction was cooled to −50° C. and NaBH₄ (75 mg, 1.98 mmol) was added portion-wise under a nitrogen stream. The mixture was warmed to rt over 1 hour. Reaction was subsequently added drop-wise to an ice water solution (15 mL) and the resulting slurry was filtered through celite CELITE®. The filtrate was extracted with EtOAc (3×3 mL) and the combined organics were further washed with brine (5 mL), dried over sodium sulfate, filtered, and concentrated under reduced pressure Crude sample was purified by reverse phase HPLC to afford (5S)-5-[[(7-bromochroman-4-yl)amino]methyl]pyrrolidin-2-one (43 mg, 20.0% yield) as a brown oil. MS: m/z found 325, 327 [M+H].

### tert-Butyl ((6-(2,2′-dichloro-3′-(4-((((S)-5-oxopyrrolidin-2-yl)methyl)amino)chroman-7-yl)-[1,1′-biphenyl]-3-yl)-2-methoxypyridin-3-yl)methyl)(((S)-5-oxopyrrolidin-2-yl)methyl)carbamate

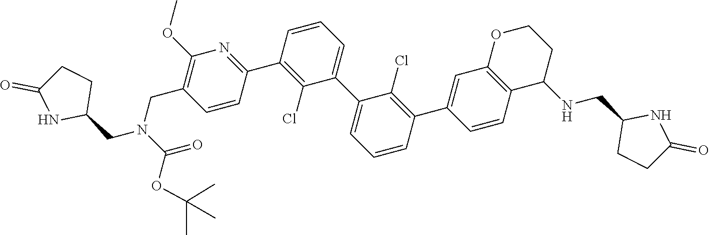 [SMILES: COc1nc(-c2cccc(-c3cccc(-c4ccc5c(c4)OCCC5NC[C@@H]4CCC(=O)N4)c3Cl)c2Cl)ccc1CN(C[C@@H]1CCC(=O)N1)C(=O)OC(C)(C)C]

Pd(PPh₃)₄ (19 mg, 0.02 mmol), potassium carbonate (33 mg, 0.24 mmol), tert-butyl N-[[6-[2-chloro-3-[2-chloro-3-(4,4,5,5-tetramethyl-1,3,2-dioxaborolan-2-yl)phenyl]phenyl]-2-methoxy-3-pyridyl]methyl]-N-[[(2S)-5-oxopyrrolidin-2-yl]methyl]carbamate (55 mg, 0.08 mmol), and (5S)-5-[[(7-bromochroman-4-yl)amino]methyl]pyrrolidin-2-one (39 mg, 0.12 mmol) were suspended in dioxane/water (4:1, 1 mL), then the solution was heated at 105° C. for 20 minutes. Reaction was cooled to rt, diluted with 3 mL water, and extracted with EtOAc (3×2 mL). The combined organics were concentrated under reduced pressure and crude sample was purified by normal phase SiO₂ chromatography (0-6% MeOH/DCM) to afford tert-butyl N-[[6-[2-chloro-3-[2-chloro-3-[4-[[(2S)-5-oxopyrrolidin-2-yl]methylamino]-chroman-7-yl]phenyl]phenyl]-2-methoxy-3-pyridyl]methyl]-N-[[(2S)-5-oxopyrrolidin-2-yl]methyl]carbamate (36.7 mg, 56.9% yield) as a tan foam. MS: m/z found 800.5, 802 [M+H]⁺.

### (5S)-5-((((6-(2,2′-Dichloro-3′-(4-((((S)-5-oxopyrrolidin-2-yl)methyl)amino)chroman-7-yl)-[1,1′-biphenyl]-3-yl)-2-methoxypyridin-3-yl)methyl)amino)methyl)pyrrolidin-2-one

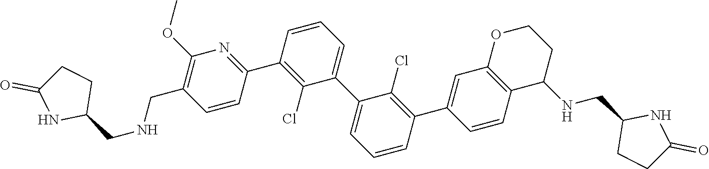 [SMILES: COc1nc(-c2cccc(-c3cccc(-c4ccc5c(c4)OCCC5NC[C@@H]4CCC(=O)N4)c3Cl)c2Cl)ccc1CNC[C@@H]1CCC(=O)N1]

tert-Butyl N-[[6-[2-chloro-3-[2-chloro-3-[4-[[(2S)-5-oxopyrrolidin-2-yl]methylamino]chroman-7-yl]phenyl]phenyl]-2-methoxy-3-pyridyl]methyl]-N-[[(2S)-5-oxopyrrolidin-2-yl]methyl]carbamate (37 mg, 0.05 mmol) was dissolved in 1 mL DCM and 4M HCl solution in dioxane (46 μL, 0.18 mmol) was added. Reaction was stirred at rt for 20 minutes and solvent was removed under reduced pressure. Crude sample was purified by reverse phase HPLC to afford (5S)-5-[[[6-[2-chloro-3-[2-chloro-3-[4-[[(2S)-5-oxopyrrolidin-2-yl]methylamino]chroman-7-yl]phenyl]phenyl]-2-methoxy-3-pyridyl]methylamino]methyl]pyrrolidin-2-one (19 mg, 60% yield) as a white solid. MS: m/z found 700.4, 702 [M+H]⁺. ¹H NMR formic acid salt (400 MHz, Methanol-d₄) δ 8.36 (s, 1H), 7.83 (d, J=7.5 Hz, 1H), 7.62 (dd, J=7.7, 1.7 Hz, 1H), 7.49 (t, J=7.6 Hz, 1H), 7.46-7.39 (m, 3H), 7.39-7.34 (m, 1H), 7.33-7.25 (m, 3H), 6.87 (d, J=8.5 Hz, 1H), 4.36 (t, J=9.4 Hz, 1H), 4.25 (d, J=11.4 Hz, 1H), 4.20-4.09 (m, 2H), 4.06 (s, 3H), 4.00 (d, J=8.1 Hz, 2H), 3.82 (s, 1H), 3.04 (t, J=5.9 Hz, 2H), 2.97-2.88 (m, 1H), 2.84 (d, J=6.2 Hz, 1H), 2.44-2.23 (m, 6H), 2.12 (s, 2H), 1.95-1.77 (m, 2H).

### Example 327: (S)-8-(2,2′-Dichloro-3′-(6-methoxy-5-((((5-oxopyrrolidin-2-yl)methyl)-amino)methyl)-pyridin-2-yl)-[1,1′-biphenyl]-3-yl)-3-((5-oxo-1,4-diazepan-1-yl)methyl)-4H-pyrido[1,2-a]pyrimidin-4-one

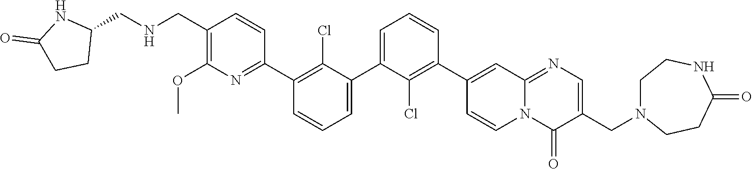 [SMILES: COc1nc(-c2cccc(-c3cccc(-c4ccn5c(=O)c(CN6CCNC(=O)CC6)cnc5c4)c3Cl)c2Cl)ccc1CNC[C@@H]1CCC(=O)N1]

Compound was prepared in the same manner as described for Example 80, utilizing intermediate (S)-tert-butyl ((6-(2,2′-dichloro-3′-(3-formyl-4-oxo-4H-pyrido[1,2-a]pyrimidin-8-yl)-[1,1′-biphenyl]-3-yl)-2-methoxypyridin-3-yl)methyl)((5-oxopyrrolidin-2-yl)methyl)carbamate. 2-aminoethan-1-ol was replaced by 1,4-diazepan-5-one. White foam. MS: m/z found 726.4, 728[M+H]⁺. ¹H NMR (400 MHz, Chloroform-d) δ 9.10 (dd, J=7.4, 0.8 Hz, 1H), 8.41 (s, 1H), 7.77 (dd, J=1.9, 0.7 Hz, 1H), 7.69 (dd, J=7.7, 1.7 Hz, 1H), 7.59 (d, J=7.5 Hz, 1H), 7.54-7.43 (m, 4H), 7.38 (dd, J=7.4, 2.0 Hz, 1H), 7.33 (dd, J=7.5, 1.7 Hz, 1H), 7.28 (s, 1H), 6.03 (s, 1H), 5.91 (t, J=5.8 Hz, 1H), 4.03 (s, 3H), 3.78 (d, J=25.9 Hz, 5H), 3.34 (q, J=6.0, 5.5 Hz, 2H), 2.84-2.72 (m, 5H), 2.70-2.63 (m, 2H), 2.57 (dd, J=11.9, 8.7 Hz, 1H), 2.37 (dd, J=8.9, 7.2 Hz, 2H), 2.25 (dt, J=12.8, 7.4 Hz, 1H), 1.84-1.71 (m, 1H).

### Example 328: (S)-3-((3,3-Bis(hydroxymethyl)pyrrolidin-1-yl)methyl)-8-(2,2′-dichloro-3′-(6-methoxy-5-((((5-oxopyrrolidin-2-yl)methyl)amino)methyl)pyridin-2-yl)-[1,1′-biphenyl]-3-yl)-4H-pyrido[1,2-a]pyrimidin-4-one

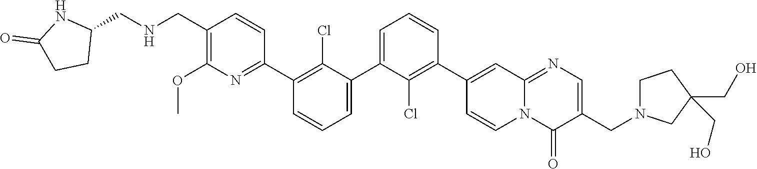 [SMILES: COc1nc(-c2cccc(-c3cccc(-c4ccn5c(=O)c(CN6CCC(CO)(CO)C6)cnc5c4)c3Cl)c2Cl)ccc1CNC[C@@H]1CCC(=O)N1]

Compound was prepared in the same manner as described for Example 80, utilizing intermediate (S)-tert-butyl ((6-(2,2′-dichloro-3′-(3-formyl-4-oxo-4H-pyrido[1,2-a]pyrimidin-8-yl)-[1,1′-biphenyl]-3-yl)-2-methoxypyridin-3-yl)methyl)((5-oxopyrrolidin-2-yl)methyl)carbamate. 2-aminoethan-1-ol was replaced by pyrrolidine-3,3-diyldimethanol. White foam. MS: m/z found 743.4, 745.4 [M+H]⁺. ¹H NMR (400 MHz, Chloroform-d) δ 9.15-9.01 (m, 1H), 8.38 (s, 1H), 7.75 (dt, J=1.7, 0.8 Hz, 1H), 7.68 (d, J=7.5 Hz, 1H), 7.58 (d, J=7.4 Hz, 1H), 7.53-7.41 (m, 4H), 7.40-7.31 (m, 2H), 7.28 (s, 1H), 6.06 (s, 1H), 4.03 (t, J=0.8 Hz, 3H), 3.81 (s, 3H), 3.74-3.65 (m, 4H), 3.62 (d, J=10.3 Hz, 2H), 2.80 (dd, J=11.9, 4.1 Hz, 1H), 2.73 (t, J=7.0 Hz, 2H), 2.65 (s, 2H), 2.56 (dd, J=12.0, 8.7 Hz, 1H), 2.36 (dd, J=9.0, 7.1 Hz, 2H), 2.24 (dq, J=14.8, 7.3 Hz, 1H), 1.79-1.75 (m, 1H), 1.69 (d, J=14.0 Hz, 2H).

### Example 329: (S)-3-((6-Acetyl-2,6-diazaspiro[3.3]heptan-2-yl)methyl)-8-(2,2′-dichloro-3′-(6-methoxy-5-((((5-oxopyrrolidin-2-yl)methyl)amino)methyl)pyridin-2-yl)-[1,1′-biphenyl]-3-yl)-4H-pyrido[1,2-a]pyrimidin-4-one

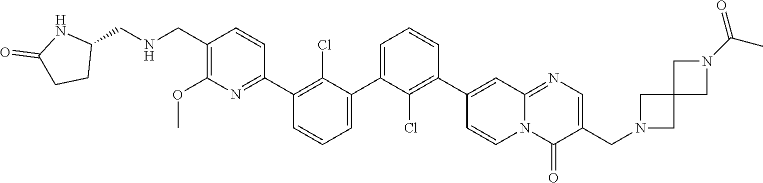 [SMILES: COc1nc(-c2cccc(-c3cccc(-c4ccn5c(=O)c(CN6CC7(C6)CN(C(C)=O)C7)cnc5c4)c3Cl)c2Cl)ccc1CNC[C@@H]1CCC(=O)N1]

Compound was prepared in the same manner as described for Example 80, utilizing intermediate (S)-tert-butyl ((6-(2,2′-dichloro-3′-(3-formyl-4-oxo-4H-pyrido[1,2-a]pyrimidin-8-yl)-[1,1′-biphenyl]-3-yl)-2-methoxypyridin-3-yl)methyl)((5-oxopyrrolidin-2-yl)methyl)carbamate. 2-aminoethan-1-ol was replaced by 1-(2,6-diazaspiro-[3.3]-heptan-2-yl)-ethan-1-one. White foam. MS: m/z found 752.4, 754 [M+H]⁺. ¹H NMR (400 MHz, Chloroform-d) δ 9.11-9.06 (m, 1H), 8.33 (s, 1H), 7.75 (t, J=1.3 Hz, 1H), 7.69 (dd, J=7.6, 1.7 Hz, 1H), 7.59 (d, J=7.5 Hz, 1H), 7.53-7.41 (m, 4H), 7.35 (ddd, J=15.3, 7.5, 1.8 Hz, 2H), 7.28 (s, 1H), 5.99 (d, J=4.0 Hz, 1H), 4.21 (s, 2H), 4.06 (s, 2H), 4.03 (s, 3H), 3.81 (s, 3H), 3.68 (s, 2H), 3.52-3.42 (m, 4H), 2.80 (dd, J=11.9, 4.2 Hz, 1H), 2.57 (dd, J=12.1, 8.6 Hz, 1H), 2.37 (dd, J=8.9, 7.1 Hz, 2H), 2.30-2.19 (m, 1H), 1.85 (s, 3H), 1.78 (dd, J=8.6, 4.5 Hz, 1H).

### Example 344: (S)-5-((((6-(2,2′-Dichloro-3′-(2,3,4,5-tetrahydro-1H-benzo[c]azepin-7-yl)-[1,1′-biphenyl]-3-yl)-2-methoxypyridin-3-yl)methyl)amino)methyl)pyrrolidin-2-one

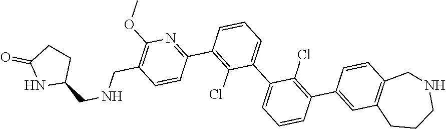 [SMILES: COc1nc(-c2cccc(-c3cccc(-c4ccc5c(c4)CCCNC5)c3Cl)c2Cl)ccc1CNC[C@@H]1CCC(=O)N1]

### tert-Butyl (S)-((6-(2,2′-dichloro-3′-(2,3,4,5-tetrahydro-1H-benzo[c]azepin-7-yl)-[1,1′-biphenyl]-3-yl)-2-methoxypyridin-3-yl)methyl)((5-oxopyrrolidin-2-yl)methyl)carbamate

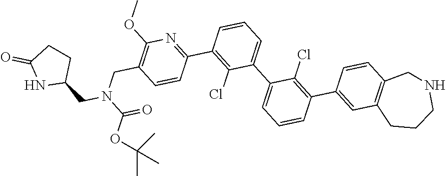 [SMILES: COc1nc(-c2cccc(-c3cccc(-c4ccc5c(c4)CCCNC5)c3Cl)c2Cl)ccc1CN(C[C@@H]1CCC(=O)N1)C(=O)OC(C)(C)C]

Pd(PPh₃)₄ (14 mg, 0.01 mmol), potassium carbonate (24 mg, 0.18 mmol), tert-butyl N-[[6-[2-chloro-3-[2-chloro-3-(4,4,5,5-tetramethyl-1,3,2-dioxaborolan-2-yl)phenyl]phenyl]-2-methoxy-3-pyridyl]methyl]-N-[[(2S)-5-oxopyrrolidin-2-yl]methyl]carbamate (40 mg, 0.06 mmol, and 7-bromo-2,3,4,5-tetrahydro-1H-2-benzazepine; hydrochloride (20 mg, 0.08 mmol) were suspended in dioxane/water (4:1, 1 mL), then the solution was heated at 105° C. for 20 minutes. Reaction was cooled to rt, diluted with 2 mL water, and extracted with EtOAc (3×2 mL). The combined organics were concentrated under reduced pressure and crude sample was purified by normal phase SiO₂ chromatography (5-20% MeOH/DCM) to afford tert-butyl N-[[6-[2-chloro-3-[2-chloro-3-(2,3,4,5-tetrahydro-1H-2-benzazepin-7-yl)phenyl]-phenyl]-2-methoxy-3-pyridyl]methyl]-N-[[(2S)-5-oxopyrrolidin-2-yl]methyl]carbamate (28 mg, 68% yield) as a white foam. MS: m/z found 701.5, 703.4 [M+H]⁺.

### (S)-5-((((6-(2,2′-Dichloro-3′-(2,3,4,5-tetrahydro-1H-benzo [c]azepin-7-yl)-[1,1′-biphenyl]-3-yl)-2-methoxypyridin-3-yl)-methyl)amino)-methyl)pyrrolidin-2-one

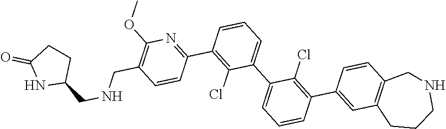 [SMILES: COc1nc(-c2cccc(-c3cccc(-c4ccc5c(c4)CCCNC5)c3Cl)c2Cl)ccc1CNC[C@@H]1CCC(=O)N1]

tert-butyl N-[[6-[2-chloro-3-[2-chloro-3-(2,3,4,5-tetrahydro-1H-2-benzazepin-7-yl)phenyl]phenyl]-2-methoxy-3-pyridyl]methyl]-N-[[(2S)-5-oxopyrrolidin-2-yl]methyl]carbamate (14 mg, 0.02 mmol) was dissolved in 0.5 mL DCM and 4M HCl solution in dioxane (20 μL, 0.08 mmol) was added. Reaction was stirred at rt for 20 minutes and solvent was removed under reduced pressure. Crude sample was purified by reverse phase HPLC to afford (5S)-5-[[[6-[2-chloro-3-[2-chloro-3-(2,3,4,5-tetrahydro-1H-2-benzazepin-7-yl)phenyl]phenyl]-2-methoxy-3-pyridyl]methylamino]methyl]pyrrolidin-2-one (9.2 mg, 76.7% yield) as a white foam. MS: m/z found 601.4, 603 [M+H]⁺. ¹H NMR formic acid salt (400 MHz, Methanol-d₄) δ 8.51-8.42 (m, 1H), 7.76 (d, J=7.4 Hz, 1H), 7.63 (dd, J=7.8, 1.7 Hz, 1H), 7.51-7.44 (m, 3H), 7.43-7.32 (m, 5H), 7.26 (d, J=7.4 Hz, 1H), 4.44 (s, 2H), 4.03 (s, 3H), 3.97-3.85 (m, 3H), 3.54-3.46 (m, 2H), 3.14 (d, J=8.0 Hz, 2H), 2.87-2.74 (m, 2H), 2.38-2.25 (m, 3H), 2.04 (s, 2H), 1.89-1.77 (m, 1H).

### Example 360: 8-(2,2′-Dichloro-3′-(5-methoxy-6-(((((S)-5-oxopyrrolidin-2-yl)-methyl)-amino)methyl)-pyridin-3-yl)-[1,1′-biphenyl]-3-yl)-3-(((((S)-5-oxopyrrolidin-2-yl)methyl)amino)methyl)-4H-pyrido[1,2-a]pyrimidin-4-one

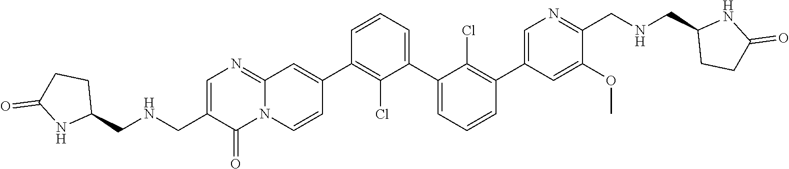 [SMILES: COc1cc(-c2cccc(-c3cccc(-c4ccn5c(=O)c(CNC[C@@H]6CCC(=O)N6)cnc5c4)c3Cl)c2Cl)cnc1CNC[C@@H]1CCC(=O)N1]

### 8-(2,2′-Dichloro-3′-(6-formyl-5-methoxypyridin-3-yl)-[1,1′-biphenyl]-3-yl)-4-oxo-4H-pyrido[1,2-a]pyrimidine-3-carbaldehyde

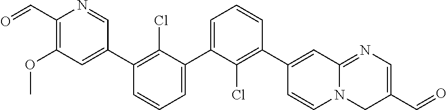 [SMILES: COc1cc(-c2cccc(-c3cccc(C4=CC5=NC=C(C=O)CN5C=C4)c3Cl)c2Cl)cnc1C=O]

Pd(PPh₃)₄ (50 mg, 0.04 mmol), potassium carbonate (100 mg, 0.72 mmol), 8-[2-chloro-3-[2-chloro-3-(4,4,5,5-tetramethyl-1,3,2-dioxaborolan-2-yl)phenyl]phenyl]-4-oxo-pyrido[1,2-a]pyrimidine-3-carbaldehyde (150 mg, 0.29 mmol), and 5-bromo-3-methoxy-pyridine-2-carbaldehyde (81 mg, 0.37 mmol) were suspended in dioxane/water (4:1, 3 mL), then the solution was heated at 105° C. for 20 minutes. Reaction was cooled to rt, diluted with 5 mL water, and extracted with EtOAc (3×3 mL). The combined organics were concentrated under reduced pressure and crude sample was purified by normal phase SiO₂ chromatography (0-60% EtOAc/hexane) to afford 8-[2-chloro-3-[2-chloro-3-(6-formyl-5-methoxy-3-pyridyl)-phenyl]-phenyl]-4-oxo-pyrido[1,2-a]pyrimidine-3-carbaldehyde (75 mg, 48.8% yield) as a yellow solid. MS: m/z found 530.1, 532.1 [M+H]⁺.

### 8-(2,2′-Dichloro-3′-(5-methoxy-6-(((((S)-5-oxopyrrolidin-2-yl)methyl)amino)methyl)pyridin-3-yl)-[1,1′-biphenyl]-3-yl)-3-(((((S)-5-oxopyrrolidin-2-yl)methyl)amino)methyl)-4H-pyrido[1,2-a]pyrimidin-4-one

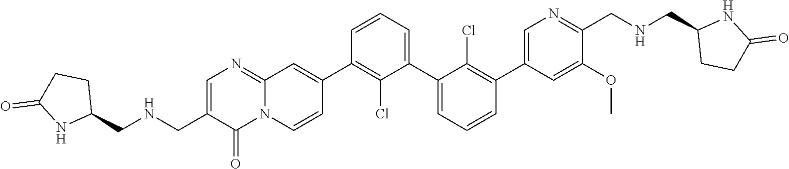 [SMILES: COc1cc(-c2cccc(-c3cccc(-c4ccn5c(=O)c(CNC[C@@H]6CCC(=O)N6)cnc5c4)c3Cl)c2Cl)cnc1CNC[C@@H]1CCC(=O)N1]

8-[2-chloro-3-[2-chloro-3-(6-formyl-5-methoxy-3-pyridyl)phenyl]phenyl]-4-oxo-pyrido[1,2-a]pyrimidine-3-carbaldehyde (25 mg, 0.05 mmol), (5S)-5-(aminomethyl)pyrrolidin-2-one (16 mg, 0.14 mmol), and acetic acid (6 mg, 0.09 mmol) were dissolved in MeOH/THF (1:1, 1 mL), then the solution was stirred at 25° C. for 2 hours. NaBH₃CN (6 mg, 0.09 mmol) was added and the resulting mixture was stirred at 25° C. for 1 hour. Reaction was subsequently diluted with water (2 mL) and extracted with EtOAc (3×2 mL). The combined organics were further washed with brine, dried over sodium sulfate, filtered, and concentrated under reduced pressure. Crude sample was purified by reverse phase HPLC to afford 8-[2-chloro-3-[2-chloro-3-[5-methoxy-6-[[[(2S)-5-oxopyrrolidin-2-yl]methylamino]methyl]-3-pyridyl]phenyl]phenyl]-3-[[[(2S)-5-oxopyrrolidin-2-yl]methylamino]methyl]pyrido[1,2-a]pyrimidin-4-one (14.8 mg, 43.2% yield) as a white solid. MS: m/z found 726.5, 728.4 [M+H]. ¹H NMR formic acid salt (400 MHz, Methanol-d₄) δ 9.16 (dd, J=7.4, 0.7 Hz, 1H), 8.45 (s, 2H), 8.26 (d, J=1.7 Hz, 1H), 7.79 (dd, J=1.9, 0.7 Hz, 1H), 7.69-7.48 (m, 8H), 7.45 (dd, J=7.0, 2.3 Hz, 1H), 4.25 (d, J=2.9 Hz, 2H), 4.00 (t, J=5.4 Hz, 3H), 3.96 (s, 3H), 3.92 (d, J=5.1 Hz, 1H), 3.10-2.97 (m, 2H), 2.88 (qd, J=12.3, 6.1 Hz, 2H), 2.43-2.27 (m, 6H), 1.96-1.78 (m, 2H).

### Example 361: 8-(2,2′-Dichloro-3′-(6-((((1r,3r)-3-hydroxy-3-methylcyclobutyl)amino)methyl)-5-methoxypyridin-3-yl)-[1,1′-biphenyl]-3-yl)-3-((((1r,3r)-3-hydroxy-3-methylcyclobutyl)amino)methyl)-4H-pyrido[1,2-a]pyrimidin-4-one

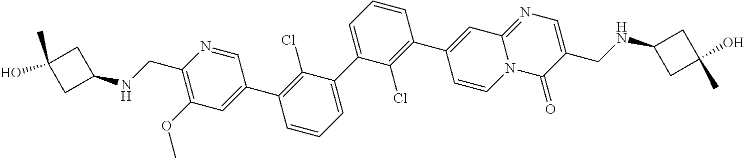 [SMILES: COc1cc(-c2cccc(-c3cccc(-c4ccn5c(=O)c(CN[C@H]6C[C@](C)(O)C6)cnc5c4)c3Cl)c2Cl)cnc1CN[C@H]1C[C@](C)(O)C1]

Compound was prepared in the same manner as described for Example 360, utilizing intermediate 8-(2,2′-dichloro-3′-(6-formyl-5-methoxypyridin-3-yl)-[1,1′-biphenyl]-3-yl)-4-oxo-4H-pyrido[1,2-a]pyrimidine-3-carbaldehyde. (S)-5-(aminomethyl)-pyrrolidin-2-one was replaced by (1R,3R)-3-amino-1-methylcyclobutan-1-ol. White solid. MS: m/z found 700.3, 702.4 [M+H]⁺. ¹H NMR formic acid salt (400 MHz, Methanol-d₄) δ 9.15 (dd, J=7.4, 0.7 Hz, 1H), 8.53 (s, 1H), 8.44 (s, 1H), 8.25 (d, J=1.7 Hz, 1H), 7.80 (dd, J=2.0, 0.7 Hz, 1H), 7.67-7.49 (m, 7H), 7.45 (dd, J=7.0, 2.3 Hz, 1H), 4.16 (s, 2H), 3.96 (d, J=6.9 Hz, 5H), 3.86 (p, J=8.0 Hz, 1H), 3.74 (p, J=7.9 Hz, 1H), 2.40-2.31 (m, 4H), 2.18-2.00 (m, 4H), 1.37 (d, J=4.7 Hz, 6H).

### Example 362: 8-(2,2′-Dichloro-3′-(6-(((2-hydroxyethyl)(methyl)amino)methyl)-5-methoxypyridin-3-yl)-[1,1′-biphenyl]-3-yl)-3-(((2-hydroxyethyl)(methyl)amino)methyl)-4H-pyrido[1,2-a]pyrimidin-4-one

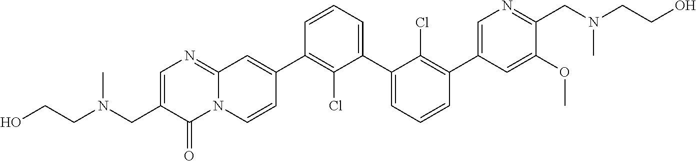 [SMILES: COc1cc(-c2cccc(-c3cccc(-c4ccn5c(=O)c(CN(C)CCO)cnc5c4)c3Cl)c2Cl)cnc1CN(C)CCO]

Compound was prepared in the same manner as described for Example 360, utilizing intermediate 8-(2,2′-dichloro-3′-(6-formyl-5-methoxypyridin-3-yl)-[1,1′-biphenyl]-3-yl)-4-oxo-4H-pyrido[1,2-a]pyrimidine-3-carbaldehyde. (S)-5-(aminomethyl)-pyrrolidin-2-one was replaced by 2-(methylamino)-ethan-1-ol. White solid. MS: m/z found 648.3, 650 [M+H]⁺. ¹H NMR formic acid salt (400 MHz, Methanol-d₄) δ 9.19 (d, J=7.4 Hz, 1H), 8.53 (s, 1H), 8.46 (s, 2H), 8.31 (d, J=1.7 Hz, 1H), 7.86 (d, J=1.8 Hz, 1H), 7.67-7.59 (m, 4H), 7.58-7.51 (m, 3H), 7.47 (dd, J=6.7, 2.6 Hz, 1H), 4.50 (s, 2H), 4.29 (s, 2H), 3.98 (s, 3H), 3.91 (dt, J=8.4, 5.3 Hz, 4H), 3.21 (t, J=5.3 Hz, 2H), 2.93 (s, 3H), 2.81 (s, 3H).

### Example 363: (S)-5-((((6-(2,2′-Dichloro-3′-(1-methyl-2,3,4,5-tetrahydro-1H-benzo[e][1,4]diazepin-7-yl)-[1,1′-biphenyl]-3-yl)-2-methoxypyridin-3-yl)methyl)amino)methyl)pyrrolidin-2-one

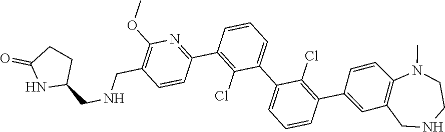 [SMILES: COc1nc(-c2cccc(-c3cccc(-c4ccc5c(c4)CNCCN5C)c3Cl)c2Cl)ccc1CNC[C@@H]1CCC(=O)N1]

### tert-Butyl 7-bromo-1-methyl-1,2,3,5-tetrahydro-4H-benzo[e][1,4]diazepine-4-carboxylate

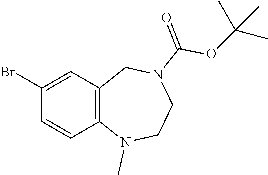 [SMILES: CN1CCN(C(=O)OC(C)(C)C)Cc2cc(Br)ccc21]

Sodium hydride (48 mg, 60% in oil dispersion, 1.19 mmol) was suspended in 6 mL anhydrous DMF and the solution was cooled to 0° C. A solution of tert-butyl 7-bromo-1,2,3,5-tetrahydro-1,4-benzodiazepine-4-carboxylate (300 mg, 0.92 mmol) in 3 mL DMF was added drop-wise over 2 minutes. The resulting mixture was warmed to rt and stirred to 30 minutes, then cooled back to 0° C. Iodomethane (80 μL, 182 mg, 1.28 mmol) was added drop-wise, then the reaction mixture was warmed to 50° C. and stirred for 12 hours. Reaction was diluted with 20 mL water and extracted with EtOAc (3×20 mL). The combined organics were concentrated under reduced pressure and the crude oil was purified by normal phase SiO₂ chromatography (0-40% EtOAc/hexane) to afford tert-butyl 7-bromo-1-methyl-3,5-dihydro-2H-1,4-benzodiazepine-4-carboxylate (164 mg, 52.4% yield) as a clear oil. MS: m/z found 341.1, 343 [M+H]⁺.

### tert-Butyl (S)-7-(3′-(5-(((tert-butoxycarbonyl)((5-oxopyrrolidin-2-yl)methyl)amino)methyl)-6-methoxypyridin-2-yl)-2,2′-dichloro-[1,1′-biphenyl]-3-yl)-1-methyl-1,2,3,5-tetrahydro-4H-benzo[e][1,4]diazepine-4-carboxylate

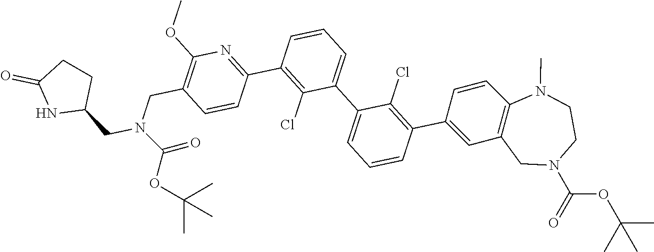 [SMILES: COc1nc(-c2cccc(-c3cccc(-c4ccc5c(c4)CN(C(=O)OC(C)(C)C)CCN5C)c3Cl)c2Cl)ccc1CN(C[C@@H]1CCC(=O)N1)C(=O)OC(C)(C)C]

Pd(PPh₃)₄ (13 mg, 0.01 mmol), potassium carbonate (22 mg, 0.16 mmol), tert-butyl N-[[6-[2-chloro-3-[2-chloro-3-(4,4,5,5-tetramethyl-1,3,2-dioxaborolan-2-yl)phenyl]phenyl]-2-methoxy-3-pyridyl]methyl]-N-[[(2S)-5-oxopyrrolidin-2-yl]methyl]carbamate (37 mg, 0.05 mmol), and tert-butyl 7-bromo-1-methyl-3,5-dihydro-2H-1,4-benzodiazepine-4-carboxylate (24 mg, 0.07 mmol) were suspended in dioxane/water (4:1, 2 mL), then the solution was heated at 105° C. for 20 minutes. Reaction was cooled to rt, diluted with 4 mL water, and extracted with EtOAc (3×2 mL). The combined organics were concentrated under reduced pressure and crude sample was purified by normal phase SiO₂ chromatography (5-20% MeOH/DCM) to afford tert-butyl 7-[3-[3-[5-[[tert-butoxycarbonyl-[[(2S)-5-oxopyrrolidin-2-yl]methyl]amino]methyl]-6-methoxy-2-pyridyl]-2-chloro-phenyl]-2-chloro-phenyl]-1-methyl-3,5-dihydro-2H-1,4-benzodiazepine-4-carboxylate (42 mg, 66.4% yield) as a white foam. MS: m/z found 816, 818.4 [M+H]⁺.

### (S)-5-((((6-(2,2′-Dichloro-3′-(1-methyl-2,3,4,5-tetrahydro-1H-benzo[e][1,4]diazepin-7-yl)-[1,1′-biphenyl]-3-yl)-2-methoxypyridin-3-yl)methyl)amino)methyl)pyrrolidin-2-one

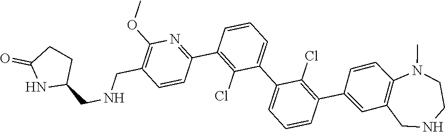 [SMILES: COc1nc(-c2cccc(-c3cccc(-c4ccc5c(c4)CNCCN5C)c3Cl)c2Cl)ccc1CNC[C@@H]1CCC(=O)N1]

tert-Butyl 7-[3-[3-[5-[[tert-butoxycarbonyl-[[(2S)-5-oxopyrrolidin-2-yl]methyl]amino]methyl]-6-methoxy-2-pyridyl]-2-chloro-phenyl]-2-chloro-phenyl]-1-methyl-3,5-dihydro-2H-1,4-benzodiazepine-4-carboxylate (42 mg, 0.04 mmol) was dissolved in 1 mL DCM and 4M HCl solution in dioxane (36 μL, 0.14 mmol) was added. Reaction was stirred at rt for 20 minutes and solvent was removed under reduced pressure. Crude sample was purified by reverse phase HPLC to afford (5S)-5-[[[6-[2-chloro-3-[2-chloro-3-(1-methyl-2,3,4,5-tetrahydro-1,4-benzodiazepin-7-yl)-phenyl]-phenyl]-2-methoxy-3-pyridyl]-methylamino]-methyl]-pyrrolidin-2-one (8.5 mg, 38.3% yield) as a white foam MS: m/z found 616.3, 618 [M+H]⁺. ¹H NMR formic acid salt (400 MHz, Methanol-d₄) δ 8.53 (s, 1H), 7.73 (d, J=7.5 Hz, 1H), 7.62 (dd, J=7.8, 1.6 Hz, 1H), 7.51-7.37 (m, 5H), 7.33 (ddd, J=12.3, 7.5, 1.8 Hz, 2H), 7.24 (d, J=7.4 Hz, 1H), 7.19 (d, J=8.4 Hz, 1H), 4.28 (s, 2H), 4.02 (s, 3H), 3.92-3.79 (m, 3H), 3.34 (s, 2H), 3.26 (s, 2H), 3.02 (s, 3H), 2.81-2.66 (m, 2H), 2.40-2.22 (m, 3H), 1.90-1.76 (m, 1H).

### Example 364: (S)-5-((((6-(2,2′-Dichloro-3′-(1-methyl-2,3,4,5-tetrahydro-1H-benzo[e][1,4]diazepin-8-yl)-[1,1′-biphenyl]-3-yl)-2-methoxypyridin-3-yl)methyl)amino)methyl)pyrrolidin-2-one

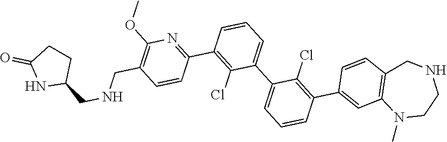 [SMILES: COc1nc(-c2cccc(-c3cccc(-c4ccc5c(c4)N(C)CCNC5)c3Cl)c2Cl)ccc1CNC[C@@H]1CCC(=O)N1]

### tert-Butyl 8-chloro-1-methyl-1,2,3,5-tetrahydro-4H-benzo[e][1,4]diazepine-4-carboxylate

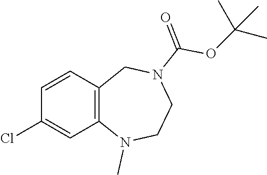 [SMILES: CN1CCN(C(=O)OC(C)(C)C)Cc2ccc(Cl)cc21]

Sodium hydride (55 mg, 60% in oil dispersion, 1.38 mmol) was suspended in 6 mL anhydrous DMF and the solution was cooled to 0° C. A solution of tert-butyl 8-chloro-1,2,3,5-tetrahydro-1,4-benzodiazepine-4-carboxylate (300 mg, 1.06 mmol) in 3 mL DMF was slowly added over 2 minutes. The resulting mixture was warmed to rt and stirred to 30 minutes, then cooled back to 0° C. Iodomethane (92 μL, 211 mg, 1.49 mmol) was added drop-wise, then the reaction mixture was warmed to 50° C. and stirred for 12 hours. Reaction was diluted with 20 mL water and extracted with EtOAc (3×20 mL). The combined organics were concentrated under reduced pressure and the crude oil was purified by normal phase SiO₂ chromatography (0-40% EtOAc/hexane) to afford tert-butyl 8-chloro-1-methyl-3,5-dihydro-2H-1,4-benzodiazepine-4-carboxylate (321 mg, 81.6% yield) as a yellow oil. MS: m/z found 297.1, 299.2 [M+H]⁺.

### tert-Butyl 1-methyl-8-(4,4,5,5-tetramethyl-1,3,2-dioxaborolan-2-yl)-1,2,3,5-tetrahydro-4H-benzo[e][1,4]diazepine-4-carboxylate

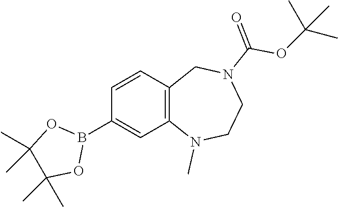 [SMILES: CN1CCN(C(=O)OC(C)(C)C)Cc2ccc(B3OC(C)(C)C(C)(C)O3)cc21]

tert-Butyl 8-chloro-1-methyl-3,5-dihydro-2H-1,4-benzodiazepine-4-carboxylate (100 mg, 0.34 mmol), 4,4,5,5-tetramethyl-2-(4,4,5,5-tetramethyl-1,3,2-dioxaborolan-2-yl)-1,3,2-dioxaborolane (128 mg, 0.51 mmol), Pd₂(dba)₃ (31 mg, 0.03 mmol), Xphos (30 mg), and potassium acetate (92 mg, 0.94 mmol) were suspended in 3 mL dioxane in a sealed tube. Nitrogen gas was bubbled through the reaction solution for 2 minutes, then the vessel was sealed and heated at 100° C. for 12 hours. Reaction was cooled to rt and the mixture was filtered through CELITE®. The filtrate was concentrated under reduced pressure and the crude oil was purified by normal phase SiO₂ chromatography (0-20% EtOAc/hexane) to afford tert-butyl 1-methyl-8-(4,4,5,5-tetramethyl-1,3,2-dioxaborolan-2-yl)-3,5-dihydro-2H-1,4-benzodiazepine-4-carboxylate (70.5 mg, 43.1% yield) as a yellow oil. MS: m/z found 389.2 [M+H]⁺.

### tert-Butyl (S)-8-(3′-(5-(((tert-butoxycarbonyl)((5-oxopyrrolidin-2-yl)methyl)amino)methyl)-6-methoxypyridin-2-yl)-2,2′-dichloro-[1,1′-biphenyl]-3-yl)-1-methyl-1,2,3,5-tetrahydro-4H-benzo[e][1,4]diazepine-4-carboxylate

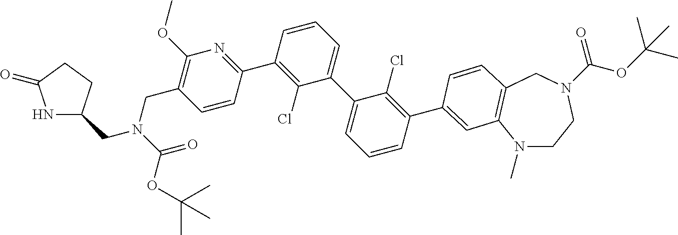 [SMILES: COc1nc(-c2cccc(-c3cccc(-c4ccc5c(c4)N(C)CCN(C(=O)OC(C)(C)C)C5)c3Cl)c2Cl)ccc1CN(C[C@@H]1CCC(=O)N1)C(=O)OC(C)(C)C]

Pd(PPh₃)₄ (15 mg, 0.01 mmol), potassium carbonate (26 mg, 0.19 mmol), tert-butyl N-[[6-[3-(3-bromo-2-chloro-phenyl)-2-chloro-phenyl]-2-methoxy-3-pyridyl]methyl]-N-[[(2S)-5-oxopyrrolidin-2-yl]methyl]carbamate (40 mg, 0.06 mmol), and tert-butyl 1-methyl-8-(4,4,5,5-tetramethyl-1,3,2-dioxaborolan-2-yl)-3,5-dihydro-2H-1,4-benzodiazepine-4-carboxylate (29 mg, 0.08 mmol) were suspended in dioxane/water (4:1, 1 mL), then the solution was heated at 105° C. for 20 minutes. Reaction was cooled to rt, diluted with 2 mL water, and extracted with EtOAc (3×2 mL). The combined organics were concentrated under reduced pressure and crude sample was purified by normal phase SiO₂ chromatography (5-100% EtOAc/hexane) to afford tert-butyl 8-[3-[3-[5-[[tert-butoxycarbonyl-[[(2S)-5-oxopyrrolidin-2-yl]methyl]amino]methyl]-6-methoxy-2-pyridyl]-2-chloro-phenyl]-2-chloro-phenyl]-1-methyl-3,5-dihydro-2H-1,4-benzodiazepine-4-carboxylate (44 mg, 42.8% yield) as a clear oil. MS: m/z found 816, 818 [M+H]⁺.

### (S)-5-((((6-(2,2′-Dichloro-3′-(1-methyl-2,3,4,5-tetrahydro-1H-benzo[e][1,4]diazepin-8-yl)-[1,1′-biphenyl]-3-yl)-2-methoxypyridin-3-yl)methyl)amino)methyl)pyrrolidin-2-one

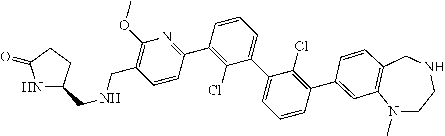 [SMILES: COc1nc(-c2cccc(-c3cccc(-c4ccc5c(c4)N(C)CCNC5)c3Cl)c2Cl)ccc1CNC[C@@H]1CCC(=O)N1]

tert-Butyl 8-[3-[3-[5-[[tert-butoxycarbonyl-[[(2S)-5-oxopyrrolidin-2-yl]-methyl]-amino]methyl]-6-methoxy-2-pyridyl]-2-chloro-phenyl]-2-chloro-phenyl]-1-methyl-3, 5-dihydro-2H-1,4-benzodiazepine-4-carboxylate (44 mg, 0.03 mmol) was dissolved in 1 mL DCM and 4M HCl solution in dioxane (27 μL, 0.11 mmol) was added. Reaction was stirred at rt for 20 minutes and solvent was removed under reduced pressure. Crude sample was purified by reverse phase HPLC to afford (5S)-5-[[[6-[2-chloro-3-[2-chloro-3-(1-methyl-2,3,4,5-tetrahydro-1,4-benzodiazepin-8-yl)phenyl]phenyl]-2-methoxy-3-pyridyl]-methylamino]methyl]-pyrrolidin-2-one (11 mg, 65.0% yield) as a white solid. MS: m/z found 616.3, 618 [M+H]⁺. ¹H NMR (400 MHz, Chloroform-d) δ 7.65 (dd, J=7.6, 1.8 Hz, 1H), 7.57 (d, J=7.5 Hz, 1H), 7.45-7.37 (m, 3H), 7.37-7.32 (m, 1H), 7.31-7.27 (m, 2H), 7.20 (d, J=7.6 Hz, 1H), 7.04 (d, J=1.6 Hz, 1H), 7.00 (dd, J=7.5, 1.6 Hz, 1H), 6.15 (s, 1H), 4.02 (s, 3H), 3.97 (s, 2H), 3.80 (s, 3H), 3.15-3.08 (m, 2H), 3.04-2.98 (m, 2H), 2.94 (s, 3H), 2.79 (dd, J=12.0, 4.2 Hz, 1H), 2.56 (ddd, J=12.1, 8.6, 1.1 Hz, 1H), 2.41-2.32 (m, 2H), 2.24 (dq, J=12.8, 7.5 Hz, 1H), 1.78-1.71 (m, 1H).

### Example 365: (S)-5-((((6-(2,2′-Dichloro-3′-(1,2,3,4-tetrahydropyrazino[1,2-a]indol-8-yl)-[1,1′-biphenyl]-3-yl)-2-methoxypyridin-3-yl)methyl)amino)methyl)919yrrolidine-2-one

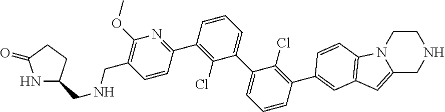 [SMILES: COc1nc(-c2cccc(-c3cccc(-c4ccc5c(c4)cc4n5CCNC4)c3Cl)c2Cl)ccc1CNC[C@@H]1CCC(=O)N1]

### tert-butyl 8-chloro-3,4-dihydropyrazino[1,2-a]-indole-2(1H)-carboxylate

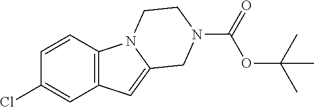 [SMILES: CC(C)(C)OC(=O)N1CCn2c(cc3cc(Cl)ccc32)C1]

8-chloro-1,2,3,4-tetrahydropyrazino[1,2-a]-indole (254 mg, 1.23 mmol), DIEA (641 μL, 476 mg, 3.69 mmol), and tert-butoxycarbonyl tert-butyl carbonate (536 mg, 2.46 mmol) were dissolved in 6 mL THF, then the mixture was stirred at rt for 12 hours. Reaction was diluted with EtOAc (10 mL) and the resulting solution was washed with saturated aqueous NaHCO₃ (5 mL×2), followed by brine (5 mL×2). The organic layer was concentrated under reduced pressure and crude sample was purified by normal phase SiO₂ chromatography (0-20% EtOAc/hexane) to afford tert-butyl 8-chloro-3,4-dihydro-1H-pyrazino[1,2-a]-indole-2-carboxylate (313 mg, 83.0% yield) as a clear oil, which solidified upon standing. MS: m/z found 307.1 [M+H]⁺.

### tert-Butyl 8-(4,4,5,5-tetramethyl-1,3,2-dioxaborolan-2-yl)-3,4-dihydropyrazino[1,2-a]indole-2(1H)-carboxylate

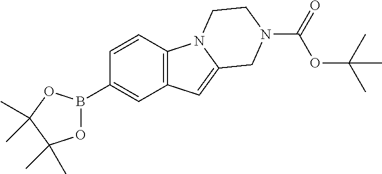 [SMILES: CC(C)(C)OC(=O)N1CCn2c(cc3cc(B4OC(C)(C)C(C)(C)O4)ccc32)C1]

tert-Butyl 8-chloro-3,4-dihydro-1H-pyrazino[1,2-a]indole-2-carboxylate (60.00 mg, 0.20 mmol), 4,4,5,5-tetramethyl-2-(4,4,5,5-tetramethyl-1,3,2-dioxaborolan-2-yl)-1,3,2-dioxaborolane (75 mg, 0.29 mmol), Pd₂(dba)₃ (18 mg, 0.02 mmol), Xphos (18 mg), and potassium acetate (54 mg, 0.55 mmol) were suspended in 3 mL dioxane in a sealed tube. Nitrogen gas was bubbled through the reaction solution for 2 minutes, then the vessel was sealed and heated at 100° C. for 12 hours. Reaction was cooled to rt and the mixture was filtered through CELITE®. The filtrate was concentrated under reduced pressure and the crude oil was purified by normal phase SiO₂ chromatography (0-20% EtOAc/hexane) to afford tert-butyl 8-(4,4,5,5-tetramethyl-1,3,2-dioxaborolan-2-yl)-3,4-dihydro-1H-pyrazino[1,2-a]indole-2-carboxylate (52 mg, 66.8% yield) as a tan solid. MS: m/z found 399.2 [M+H]⁺.

### tert-Butyl (S)-8-(3′-(5-(((tert-butoxycarbonyl)((5-oxopyrrolidin-2-yl)methyl)amino)methyl)-6-methoxypyridin-2-yl)-2,2′-dichloro-[1,1′-biphenyl]-3-yl)-3,4-dihydropyrazino[1,2-a]indole-2(1H)-carboxylate

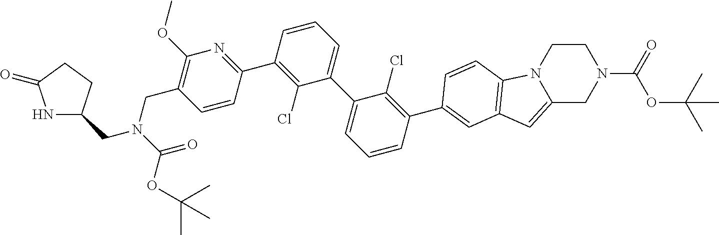 [SMILES: COc1nc(-c2cccc(-c3cccc(-c4ccc5c(c4)cc4n5CCN(C(=O)OC(C)(C)C)C4)c3Cl)c2Cl)ccc1CN(C[C@@H]1CCC(=O)N1)C(=O)OC(C)(C)C]

Pd(PPh₃)₄ (13 mg, 0.01 mmol), potassium carbonate (23 mg, 0.17 mmol), tert-butyl N-[[6-[3-(3-bromo-2-chloro-phenyl)-2-chloro-phenyl]-2-methoxy-3-pyridyl]methyl]-N-[[(2S)-5-oxopyrrolidin-2-yl]methyl]-carbamate (35 mg, 0.06 mmol), and tert-butyl 8-(4,4,5,5-tetramethyl-1,3,2-dioxaborolan-2-yl)-3,4-dihydro-1H-pyrazino[1,2-a]indole-2-carboxylate (26 mg, 0.07 mmol) were suspended in dioxane/water (4:1, 2 mL), then the solution was heated at 120° C. for 20 minutes. Reaction was cooled to rt, diluted with 3 mL water, and extracted with EtOAc (3×2 mL). The combined organics were concentrated under reduced pressure and crude sample was purified by normal phase SiO₂ chromatography (10-100% EtOAc/hexane) to afford tert-butyl 8-[3-[3-[5-[[tert-butoxycarbonyl-[[(2S)-5-oxopyrrolidin-2-yl]methyl]amino]methyl]-6-methoxy-2-pyridyl]-2-chloro-phenyl]-2-chloro-phenyl]-3,4-dihydro-1H-pyrazino[1,2-a]indole-2-carboxylate (52 mg, 57% yield) as a grey foam. MS: m/z found 826, 828 [M+H]⁺.

### (S)-5-((((6-(2,2′-Dichloro-3′-(1,2,3,4-tetrahydropyrazino[1,2-a]indol-8-yl)-[1,1′-biphenyl]-3-yl)-2-methoxypyridin-3-yl)methyl)amino)methyl)pyrrolidin-2-one

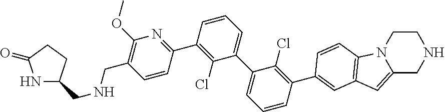 [SMILES: COc1nc(-c2cccc(-c3cccc(-c4ccc5c(c4)cc4n5CCNC4)c3Cl)c2Cl)ccc1CNC[C@@H]1CCC(=O)N1]

tert-Butyl 8-[3-[3-[5-[[tert-butoxycarbonyl-[[(2S)-5-oxopyrrolidin-2-yl]methyl]amino]methyl]-6-methoxy-2-pyridyl]-2-chloro-phenyl]-2-chloro-phenyl]-3,4-dihydro-1H-pyrazino[1,2-a]indole-2-carboxylate (52 mg, 0.03 mmol) was dissolved in 2 mL DCM and 4M HCl solution in dioxane (31 μL, 4.5 mg, 0.13 mmol) was added. Reaction was stirred at rt for 20 minutes and solvent was removed under reduced pressure. Crude sample was purified by reverse phase HPLC to afford (5S)-5-[[[6-[2-chloro-3-[2-chloro-3-(1,2,3,4-tetrahydropyrazino[1,2-a]indol-8-yl)phenyl]phenyl]-2-methoxy-3-pyridyl]methylamino]methyl]pyrrolidin-2-one (10 mg, 49% yield) as a white foam. MS: m/z found 626.3, 628 [M+H]⁺. ¹H NMR (400 MHz, Chloroform-d) δ 7.68-7.62 (m, 2H), 7.58 (d, J=7.4 Hz, 1H), 7.47-7.38 (m, 2H), 7.36 (dq, J=7.6, 1.6 Hz, 2H), 7.34-7.26 (m, 4H), 6.25 (s, 1H), 6.13 (s, 1H), 4.25 (s, 2H), 4.07 (t, J=5.7 Hz, 2H), 4.03 (d, J=1.3 Hz, 3H), 3.80 (d, J=14.5 Hz, 3H), 3.39 (t, J=5.7 Hz, 2H), 2.80 (dd, J=12.0, 4.1 Hz, 1H), 2.57 (dd, J=12.0, 8.7 Hz, 1H), 2.36 (t, J=8.1 Hz, 2H), 2.24 (dq, J=14.8, 7.5 Hz, 1H).

### Example 366: (S)-5-((((3-Chloro-5-(2,2′-dichloro-3′-(6-methoxy-5-(((((S)-5-oxopyrrolidin-2-yl)methyl)-amino)methyl)-pyridin-2-yl)-[1,1′-biphenyl]-3-yl)-1-methyl-1H-pyrrolo[3,2-b]pyridin-2-yl)methyl)amino)-methyl)-pyrrolidin-2-one

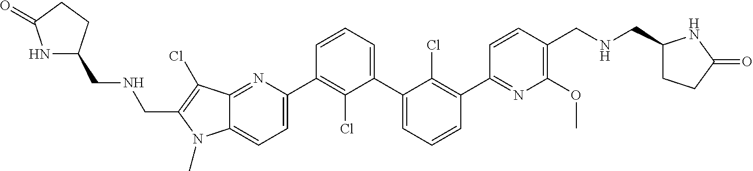 [SMILES: COc1nc(-c2cccc(-c3cccc(-c4ccc5c(n4)c(Cl)c(CNC[C@@H]4CCC(=O)N4)n5C)c3Cl)c2Cl)ccc1CNC[C@@H]1CCC(=O)N1]

### Ethyl 5-bromo-3-chloro-1H-pyrrolo[3,2-b]pyridine-2-carboxylate

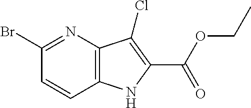 [SMILES: CCOC(=O)c1[nH]c2ccc(Br)nc2c1Cl]

A solution of ethyl 5-bromo-1H-pyrrolo[3,2-b]-pyridine-2-carboxylate (100 mg, 0.37 mmol) in 4 mL carbon tetrachloride was cooled to 0° C. 1-chloropyrrolidine-2,5-dione (55 mg, 0.41 mmol) and 2-[(E)-(1-cyano-1-methyl-ethyl)-azo]-2-methyl-propanenitrile (6 mg, 0.04 mmol) were sequentially added under a nitrogen stream, then the mixture was warmed to 80° C. and stirred for 12 hours. Reaction was cooled to rt, diluted with 5 mL DCM, and washed with water (2×5 mL), brine (2×5 mL). The organic partition was dried over sodium sulfate, filtered and concentrated under reduced pressure to afford ethyl 5-bromo-3-chloro-1H-pyrrolo[3,2-b]pyridine-2-carboxylate (112 mg, crude) as a tan solid. MS: m/z found 303, 305 [M+H]⁺. Material was used for the next step without further purification.

### Ethyl 5-bromo-3-chloro-1-methyl-11H-pyrrolo[3,2-b]pyridine-2-carboxylate

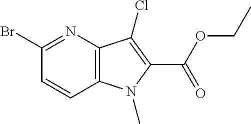 [SMILES: CCOC(=O)c1c(Cl)c2nc(Br)ccc2n1C]

Sodium hydride (19 mg, 60% in oil dispersion, 0.48 mmol) was suspended in 1 mL anhydrous DMF and the solution was cooled to 0° C. A solution of ethyl 5-bromo-3-chloro-1H-pyrrolo[3,2-b]pyridine-2-carboxylate (112 mg, 0.37 mmol) in 1 mL DMF was slowly added over 2 minutes. The resulting mixture was warmed to rt and stirred to 30 minutes, then cooled back to 0° ° C. Iodomethane (32 μL, 73 mg, 0.52 mmol) was added drop-wise, then the reaction mixture was warmed to rt and stirred for 12 hours. Reaction was diluted with 3 mL water and extracted with EtOAc (3×2 mL). The combined organics were washed with brine (2 mL), dried over sodium sulfate, filtered, then concentrated under reduced pressure to afford ethyl 5-bromo-3-chloro-1-methyl-pyrrolo[3,2-b]pyridine-2-carboxylate (112.00 mg, crude) as a tan solid. MS: m/z found 317, 319 [M+H]⁺. Material was used for the next step without further purification.

### (5-Bromo-3-chloro-1-methyl-11H-pyrrolo[3,2-b]-pyridin-2-yl)-methanol

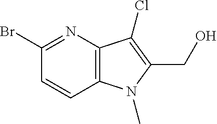

Ethyl 5-bromo-3-chloro-1-methyl-pyrrolo[3,2-b]-pyridine-2-carboxylate (112 mg, 0.35 mmol) was dissolved in 3 mL THE and the solution was cooled to −10° C. A solution of diisobutylaluminium hydride (810 μL, 1 M in THF, 0.81 mmol) was added drop-wise over 15 minutes and the resulting mixture was stirred at −10° C. for 1 hour. The solution was then warmed to rt and stirred for an additional 12 hours. Reaction was cooled to 0° C., quenched by slow addition of 5 mL water, and the solution was further stirred for 1 hour at rt. The aqueous was extracted with EtOAc (3×5 mL) and the combined organics were further washed with brine (2 mL), dried over sodium sulfate, filtered, then concentrated under reduced pressure to afford (5-bromo-3-chloro-1-methyl-pyrrolo[3,2-b]pyridin-2-yl)methanol (80 mg, crude) as a light yellow foam. MS: m/z found 275, 277 [M+H]⁺. Material was used for the next step without further purification.

### 5-Bromo-3-chloro-1-methyl-1H-pyrrolo[3,2-b]-pyridine-2-carbaldehyde

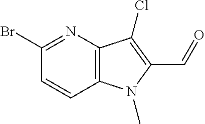

Dess-Martin periodinane (0.25 g, 0.58 mmol) was added portion-wise to a solution of (5-bromo-3-chloro-1-methyl-pyrrolo[3,2-b]pyridin-2-yl)-methanol (80 mg, 0.29 mmol) in 5 mL DCM/0.1 mL DMF and the resulting solution was stirred at rt for 2 hours. Reaction was filtered through CELITE® and the filtrate was concentrated under reduced pressure. Crude sample was purified by normal phase SiO₂ chromatography (0-15% EtOAc/hexane) to afford 5-bromo-3-chloro-1-methyl-pyrrolo[3,2-b]pyridine-2-carbaldehyde (40 mg, 50% yield) as a white solid. MS: m/z found 273, 275 [M+H]⁺.

### 3-chloro-5-(2,2′-Dichloro-3′-(5-formyl-6-methoxypyridin-2-yl)-[1,1′-biphenyl]-3-yl)-1-methyl-1H-pyrrolo[3,2-b]-pyridine-2-carbaldehyde

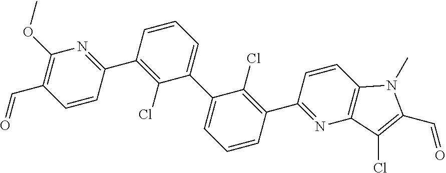 [SMILES: COc1nc(-c2cccc(-c3cccc(-c4ccc5c(n4)c(Cl)c(C=O)n5C)c3Cl)c2Cl)ccc1C=O]

Pd(PPh₃)₄ (17 mg, 0.01 mmol), potassium carbonate (30 mg, 0.22 mmol), 6-[2-chloro-3-[2-chloro-3-(4,4,5,5-tetramethyl-1,3,2-dioxaborolan-2-yl)phenyl]phenyl]-2-methoxy-pyridine-3-carbaldehyde (35 mg, 0.07 mmol), and 5-bromo-3-chloro-1-methyl-pyrrolo[3,2-b]pyridine-2-carbaldehyde (27 mg, 0.08 mmol) were suspended in dioxane/water (4:1, 1 mL), then the solution was heated at 110° C. for 20 minutes. Reaction was cooled to rt, diluted with 3 mL water, and extracted with EtOAc (3×2 mL). The combined organics were concentrated under reduced pressure and crude sample was purified by normal phase SiO₂ chromatography (0-30% EtOAc/hexane) to afford 3-chloro-5-[2-chloro-3-[2-chloro-3-(5-formyl-6-methoxy-2-pyridyl)-phenyl]-phenyl]-1-methyl-pyrrolo[3,2-b]-pyridine-2-carbaldehyde (18 mg, 45% yield) as a white foam. MS: m/z found 550.1, 552.1 [M+H]⁺.

### (S)-5-((((3-Chloro-5-(2,2′-dichloro-3′-(6-methoxy-5-(((((S)-5-oxopyrrolidin-2-yl)methyl)-amino)methyl)-pyridin-2-yl)-[1,1′-biphenyl]-3-yl)-1-methyl-11H-pyrrolo[3,2-b]pyridin-2-yl)methyl)amino)-methyl)-pyrrolidin-2-one

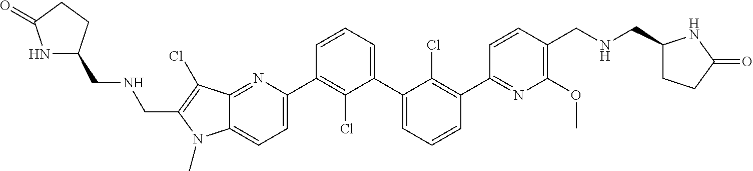 [SMILES: COc1nc(-c2cccc(-c3cccc(-c4ccc5c(n4)c(Cl)c(CNC[C@@H]4CCC(=O)N4)n5C)c3Cl)c2Cl)ccc1CNC[C@@H]1CCC(=O)N1]

3-cCloro-5-[2-chloro-3-[2-chloro-3-(5-formyl-6-methoxy-2-pyridyl)-phenyl]-phenyl]-1-methyl-pyrrolo[3,2-b]pyridine-2-carbaldehyde (18 mg, 0.03 mmol), (5S)-5-(aminomethyl)pyrrolidin-2-one (11 mg, 0.10 mmol), and acetic acid (4 mg, 0.06 mmol) were dissolved in MeOH/THF (1:1, 1 mL), then the solution was stirred at 25° C. for 2 hours. NaBH₃CN (4 mg, 0.06 mmol) was added and the resulting mixture was stirred at 25° C. for 1 hour. Reaction was subsequently diluted with water (2 mL) and extracted with EtOAc (3×2 mL). The combined organics were further washed with brine, dried over sodium sulfate, filtered, and concentrated under reduced pressure. Crude sample was purified by reverse phase HPLC to afford (5S)-5-[[[6-[2-chloro-3-[2-chloro-3-[3-chloro-1-methyl-2-[[[(2S)-5-oxopyrrolidin-2-yl]-methylamino]-methyl]-pyrrolo[3,2-b]-pyridin-5-yl]phenyl]-phenyl]-2-methoxy-3-pyridyl]methylamino]methyl]pyrrolidin-2-one (9.3 mg, 39% yield) as a white foam. MS: m/z found 748 [M+H]. ¹H NMR (400 MHz, Chloroform-d) δ 7.73-7.67 (m, 2H), 7.65 (dd, J=7.7, 1.8 Hz, 1H), 7.60-7.57 (m, 1H), 7.57-7.54 (m, 1H), 7.43 (dt, J=8.5, 7.6 Hz, 2H), 7.36-7.30 (m, 2H), 7.30-7.27 (m, 1H), 6.10 (s, 1H), 6.00 (s, 1H), 4.20-4.04 (m, 2H), 4.02 (s, 3H), 3.87 (s, 3H), 3.84-3.68 (m, 4H), 2.88 (dd, J=12.0, 3.8 Hz, 1H), 2.78 (dd, J=12.0, 4.2 Hz, 1H), 2.66-2.50 (m, 2H), 2.35 (dt, J=9.7, 6.7 Hz, 4H), 2.23 (tt, J=14.3, 7.4 Hz, 2H), 1.82-1.70 (m, 2H).

### Example 385: (S)-5-((((3-Chloro-5-(2,2′-dichloro-3′-(6-methoxy-5-(((((S)-5-oxopyrrolidin-2-yl)methyl)-amino)methyl)-pyridin-2-yl)-[1,1′-biphenyl]-3-yl)-1H-pyrrolo[3,2-b]-pyridin-2-yl)methyl)-amino)methyl)-pyrrolidin-2-one

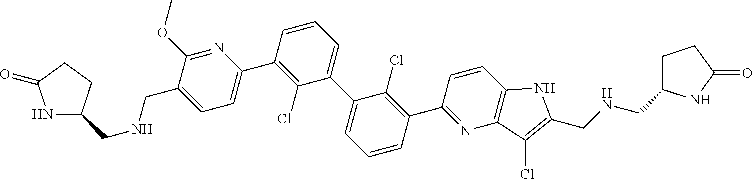 [SMILES: COc1nc(-c2cccc(-c3cccc(-c4ccc5[nH]c(CNC[C@@H]6CCC(=O)N6)c(Cl)c5n4)c3Cl)c2Cl)ccc1CNC[C@@H]1CCC(=O)N1]

### (5-Bromo-3-chloro-11H-pyrrolo[3,2-b]pyridin-2-yl)methanol

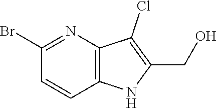 [SMILES: OCc1[nH]c2ccc(Br)nc2c1Cl]

Ethyl 5-bromo-3-chloro-1H-pyrrolo[3,2-b]pyridine-2-carboxylate (0.23 g, 0.74 mmol) was dissolved in 3 mL THF and the solution was cooled to −10° C. A solution of diisobutylaluminium hydride (2.22 m, 1 M in THF, 2.22 mmol) was added drop-wise over 15 minutes and the resulting mixture was stirred at −10° C. for 1 hour. The solution was then warmed to rt and stirred for an additional 12 hours. Reaction was cooled to 0° C., quenched by slow addition of 7 mL water, and the solution was further stirred for 1 hour at rt. The aqueous was extracted with EtOAc (3×5 mL) and the combined organics were further washed with brine (2 mL), dried over sodium sulfate, filtered, then concentrated under reduced pressure to afford (5-bromo-3-chloro-1H-pyrrolo[3,2-b]pyridin-2-yl)methanol (0.19 g crude) as a light yellow solid. MS: m/z found 261, 263 [M+H]⁺. Material was used for the next step without further purification.

### 5-Bromo-3-chloro-1H-pyrrolo[3,2-b]pyridine-2-carbaldehyde

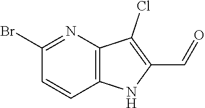 [SMILES: O=Cc1[nH]c2ccc(Br)nc2c1Cl]

Dess-Martin periodinane (0.40 g, 0.95 mmol) was added portion-wise to a solution of (5-bromo-3-chloro-1H-pyrrolo[3,2-b]pyridin-2-yl)methanol (0.19 g, 0.73 mmol) in 8 mL DCM/0.1 mL DMF and the resulting solution was stirred at rt for 2 hours. Reaction was filtered through CELITE® and the filtrate was concentrated under reduced pressure. Crude sample was purified by normal phase SiO₂ chromatography (0-30% EtOAc/hexane) to afford 5-bromo-3-chloro-1H-pyrrolo[3,2-b]pyridine-2-carbaldehyde (0.10 g, 53% yield) as a white solid. MS: m/z found 259, 261 [M+H]⁺.

### tert-Butyl (S)-((6-(2,2′-dichloro-3′-(3-chloro-2-formyl-1H-pyrrolo[3,2-b]pyridin-5-yl)-[1,1′-biphenyl]-3-yl)-2-methoxypyridin-3-yl)methyl)((5-oxopyrrolidin-2-yl)methyl)carbamate

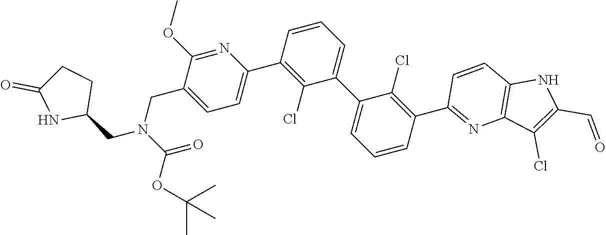 [SMILES: COc1nc(-c2cccc(-c3cccc(-c4ccc5[nH]c(C=O)c(Cl)c5n4)c3Cl)c2Cl)ccc1CN(C[C@@H]1CCC(=O)N1)C(=O)OC(C)(C)C]

Pd(PPh₃)₄ (24 mg, 0.02 mmol), potassium carbonate (42 mg, 0.31 mmol), tert-butyl N-[[6-[2-chloro-3-[2-chloro-3-(4,4,5,5-tetramethyl-1,3,2-dioxaborolan-2-yl)phenyl]phenyl]-2-methoxy-3-pyridyl]methyl]-N-[[(2S)-5-oxopyrrolidin-2-yl]methyl]carbamate (70 mg, 0.10 mmol), and 5-bromo-3-chloro-1H-pyrrolo[3,2-b]pyridine-2-carbaldehyde (35 mg, 0.13 mmol) were suspended in dioxane/water (4:1, 2 mL), then the solution was heated at 105° C. for 20 minutes. Reaction was cooled to rt, diluted with 3 mL water, and extracted with EtOAc (3×2 mL). The combined organics were concentrated under reduced pressure and crude sample was purified by normal phase SiO₂ chromatography (10-100% EtOAc/hexane) to afford tert-butyl N-[[6-[2-chloro-3-[2-chloro-3-(3-chloro-2-formyl-1H-pyrrolo[3,2-b]pyridin-5-yl)phenyl]phenyl]-2-methoxy-3-pyridyl]methyl]-N-[[(2S)-5-oxopyrrolidin-2-yl]methyl]carbamate (66 mg, 48% yield) as a yellow foam. MS: m/z found 736 [M+H]⁺.

### tert-Butyl ((6-(2,2′-dichloro-3′-(3-chloro-2-(((((S)-5-oxopyrrolidin-2-yl)methyl)amino)methyl)-1H-pyrrolo[3,2-b]pyridin-5-yl)-[1,1′-biphenyl]-3-yl)-2-methoxypyridin-3-yl)methyl)(((S)-5-oxopyrrolidin-2-yl)methyl)carbamate

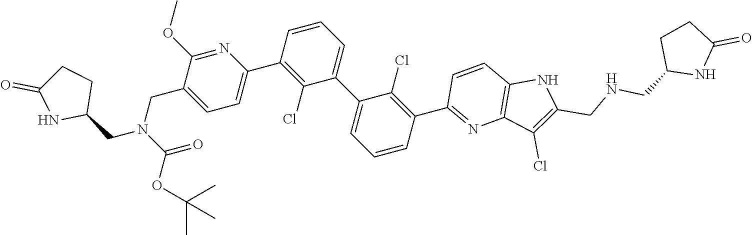 [SMILES: COc1nc(-c2cccc(-c3cccc(-c4ccc5[nH]c(CNC[C@@H]6CCC(=O)N6)c(Cl)c5n4)c3Cl)c2Cl)ccc1CN(C[C@@H]1CCC(=O)N1)C(=O)OC(C)(C)C]

tert-Butyl N-[[6-[2-chloro-3-[2-chloro-3-(3-chloro-2-formyl-1H-pyrrolo[3,2-b]pyridin-5-yl)phenyl]phenyl]-2-methoxy-3-pyridyl]methyl]-N-[[(2S)-5-oxopyrrolidin-2-yl]methyl]carbamate (33 mg, 0.02 mmol), (5S)-5-(aminomethyl)pyrrolidin-2-one (6 mg, 0.05 mmol), and acetic acid (3 mg, 0.05 mmol) were dissolved in MeOH/THF (1:1, 1 mL), then the solution was stirred at 25° C. for 2 hours. NaBH₃CN (3.10 mg, 0.05 mmol) was added and the resulting mixture was stirred at 25° C. for 1 hour. Reaction was subsequently diluted with water (2 mL) and extracted with DCM (3×2 mL). The combined organics were further washed with brine (2 mL), dried over sodium sulfate, filtered, and concentrated under reduced pressure. Crude sample was purified by reverse phase HPLC to afford tert-butyl N-[[6-[2-chloro-3-[2-chloro-3-[3-chloro-2-[[[(2S)-5-oxopyrrolidin-2-yl]methylamino]methyl]-1H-pyrrolo[3,2-b]pyridin-5-yl]phenyl]phenyl]-2-methoxy-3-pyridyl]methyl]-N-[[(2S)-5-oxopyrrolidin-2-yl]methyl]carbamate (20 mg, 39% yield) as a white foam. MS: m/z found 833, 835 [M+H]⁺.

### (S)-5-((((3-Chloro-5-(2,2′-dichloro-3′-(6-methoxy-5-(((((S)-5-oxopyrrolidin-2-yl)methyl)-amino)methyl)-pyridin-2-yl)-[1,1′-biphenyl]-3-yl)-1H-pyrrolo[3,2-b]-pyridin-2-yl)methyl)-amino)methyl)-pyrrolidin-2-one

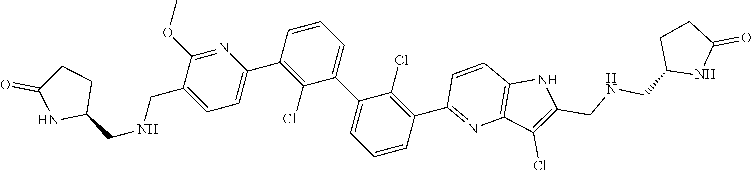 [SMILES: COc1nc(-c2cccc(-c3cccc(-c4ccc5[nH]c(CNC[C@@H]6CCC(=O)N6)c(Cl)c5n4)c3Cl)c2Cl)ccc1CNC[C@@H]1CCC(=O)N1]

tert-Butyl N-[[6-[2-chloro-3-[2-chloro-3-[3-chloro-2-[[[(2S)-5-oxopyrrolidin-2-yl]-methylamino]methyl]-1H-pyrrolo[3,2-b]pyridin-5-yl]phenyl]phenyl]-2-methoxy-3-pyridyl]methyl]-N-[[(2S)-5-oxopyrrolidin-2-yl]methyl]carbamate (20 mg, 0.01 mmol) was dissolved in 1 mL DCM and 4M HCl solution in dioxane (10 μL, 0.04 mmol) was added. Reaction was stirred at rt for 20 minutes and solvent was removed under reduced pressure. Crude sample was purified by reverse phase HPLC to afford (5S)-5-[[[6-[2-chloro-3-[2-chloro-3-[3-chloro-2-[[[(2S)-5-oxopyrrolidin-2-yl]methylamino]-methyl]-1H-pyrrolo[3,2-b]pyridin-5-yl]phenyl]phenyl]-2-methoxy-3-pyridyl]methylamino]methyl]pyrrolidin-2-one (3.8 mg, 54.0% yield) as a white solid. MS: m/z found 734.2 [M+H]⁺. ¹H NMR formic acid salt (400 MHz, Methanol-d₄) δ 8.48 (s, 2H), 7.87 (d, J=8.4 Hz, 1H), 7.76 (d, J=7.5 Hz, 1H), 7.61 (ddd, J=15.8, 7.7, 1.7 Hz, 2H), 7.50 (dt, J=9.2, 7.6 Hz, 2H), 7.44-7.37 (m, 3H), 7.28 (d, J=7.4 Hz, 1H), 4.09 (d, J=4.1 Hz, 2H), 4.03 (d, J=4.6 Hz, 3H), 3.95 (d, J=3.4 Hz, 2H), 3.90 (d, J=8.3 Hz, 1H), 3.85-3.77 (m, 1H), 2.83 (t, J=6.2 Hz, 2H), 2.76 (dd, J=12.1, 4.9 Hz, 1H), 2.64 (dd, J=12.0, 7.4 Hz, 1H), 2.39-2.20 (m, 6H), 1.83 (dq, J=11.0, 6.9, 6.3 Hz, 2H).

### Example 386: (S)-5-((((3-Chloro-6-(2,2′-dichloro-3′-(6-methoxy-5-(((((S)-5-oxopyrrolidin-2-yl)methyl)-amino)methyl)-pyridin-2-yl)-[1,1′-biphenyl]-3-yl)-1H-pyrrolo[2,3-b]-pyridin-2-yl)methyl)-amino)methyl)-pyrrolidin-2-one

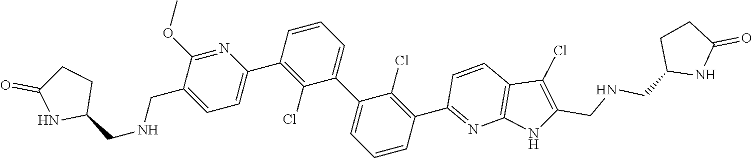 [SMILES: COc1nc(-c2cccc(-c3cccc(-c4ccc5c(Cl)c(CNC[C@@H]6CCC(=O)N6)[nH]c5n4)c3Cl)c2Cl)ccc1CNC[C@@H]1CCC(=O)N1]

### Ethyl 6-bromo-3-chloro-1H-pyrrolo[2,3-b]pyridine-2-carboxylate

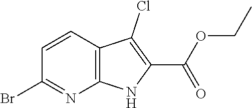 [SMILES: CCOC(=O)c1[nH]c2nc(Br)ccc2c1Cl]

A solution of ethyl 6-bromo-1H-pyrrolo[2,3-b]pyridine-2-carboxylate (0.20 g, 0.74 mmol) in 6 mL carbon tetrachloride was cooled to 0° C. 1-chloropyrrolidine-2,5-dione (0.11 g, 0.82 mmol) and 2-[(E)-(1-cyano-1-methyl-ethyl)azo]-2-methyl-propanenitrile (12 mg, 0.07 mmol) were sequentially added under a nitrogen stream, then the mixture was warmed to 80° C. and stirred for 12 hours. Reaction was cooled to rt, diluted with 8 mL DCM, and washed with water (2×5 mL), brine (2×5 mL). The organic partition was dried over sodium sulfate, filtered and concentrated under reduced pressure to afford ethyl 6-bromo-3-chloro-1H-pyrrolo[2,3-b]pyridine-2-carboxylate (225 mg, crude) as a yellow solid. MS: m/z found 303, 305 [M+H]⁺. Material was used for the next step without further purification.

### (6-Bromo-3-chloro-11H-pyrrolo[2,3-b]pyridin-2-yl)methanol

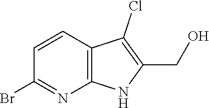 [SMILES: OCc1[nH]c2nc(Br)ccc2c1Cl]

Ethyl 6-bromo-3-chloro-1H-pyrrolo[2,3-b]pyridine-2-carboxylate (0.23 g, 0.74 mmol) was dissolved in 3 mL THF and the solution was cooled to −10° C. A solution of diisobutylaluminium hydride (2.22 mL, 1M in THF, 2.22 mmol) was added drop-wise over 15 minutes and the resulting mixture was stirred at −10° C. for 1 hour. The solution was then warmed to rt and stirred for an additional 12 hours. Reaction was cooled to 0° C., quenched by slow addition of 10 mL water, and the solution was further stirred for 1 hour at rt. The aqueous was extracted with EtOAc (3×5 mL) and the combined organics were further washed with brine (3 mL), dried over sodium sulfate, filtered, then concentrated under reduced pressure to afford (6-bromo-3-chloro-1H-pyrrolo[2,3-b]pyridin-2-yl)methanol (188.00 mg, crude) MS: m/z found 261, 263 [M+H]⁺. Material was used for the next step without further purification.

### 6-Bromo-3-chloro-1H-pyrrolo[2,3-b]pyridine-2-carbaldehyde

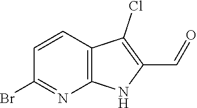 [SMILES: O=Cc1[nH]c2nc(Br)ccc2c1Cl]

Dess-Martin periodinane (0.40 g, 0.93 mmol) was added portion-wise to a solution of (6-bromo-3-chloro-1H-pyrrolo[2,3-b]pyridin-2-yl)methanol (0.19 g, 0.72 mmol) in 8 mL DCM/0.1 mL DMF and the resulting solution was stirred at rt for 2 hours. Reaction was filtered through CELITE® and the filtrate was concentrated under reduced pressure. Crude sample was purified by normal phase SiO₂ chromatography (0-30% EtOAc/hexane) to afford 6-bromo-3-chloro-1H-pyrrolo[2,3-b]pyridine-2-carbaldehyde (0.11 g, 61% yield) as a white solid. MS: m/z found 259, 261 [M+H]⁺.

### tert-Butyl (S)-((6-(2,2′-dichloro-3′-(3-chloro-2-formyl-1H-pyrrolo[2,3-b]pyridin-6-yl)-[1,1′-biphenyl]-3-yl)-2-methoxypyridin-3-yl)methyl)((5-oxopyrrolidin-2-yl)methyl)carbamate

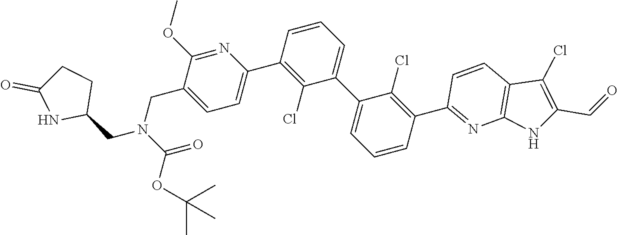 [SMILES: COc1nc(-c2cccc(-c3cccc(-c4ccc5c(Cl)c(C=O)[nH]c5n4)c3Cl)c2Cl)ccc1CN(C[C@@H]1CCC(=O)N1)C(=O)OC(C)(C)C]

Pd(PPh₃)₄ (24 mg, 0.02 mmol), potassium carbonate (44 mg, 0.32 mmol), tert-butyl N-[[6-[2-chloro-3-[2-chloro-3-(4,4,5,5-tetramethyl-1,3,2-dioxaborolan-2-yl)phenyl]phenyl]-2-methoxy-3-pyridyl]methyl]-N-[[(2S)-5-oxopyrrolidin-2-yl]methyl]carbamate (72 mg, 0.11 mmol), and 6-bromo-3-chloro-1H-pyrrolo[2,3-b]pyridine-2-carbaldehyde (36 mg, 0.14 mmol) were suspended in dioxane/water (4:1, 2 mL), then the solution was heated at 105° C. for 20 minutes. Reaction was cooled to rt, diluted with 3 ml water, and extracted with EtOAc (3×2 mL). The combined organics were concentrated under reduced pressure and crude sample was purified by normal phase SiO₂ chromatography (10-100% EtOAc/hexane) to afford tert-butyl N-[[6-[2-chloro-3-[2-chloro-3-(3-chloro-2-formyl-1H-pyrrolo[2,3-b]pyridin-6-yl)phenyl]phenyl]-2-methoxy-3-pyridyl]methyl]-N-[[(2S)-5-oxopyrrolidin-2-yl]methyl]carbamate (57 mg, 73.0% yield) as a yellow foam. MS: m/z found 736 [M+H]⁺.

### tert-Butyl ((6-(2,2′-dichloro-3′-(3-chloro-2-(((((S)-5-oxopyrrolidin-2-yl)methyl)amino)-methyl)-1H-pyrrolo-[2,3-b]pyridin-6-yl)-[1,1′-biphenyl]-3-yl)-2-methoxypyridin-3-yl)methyl)(((S)-5-oxopyrrolidin-2-yl)methyl)carbamate

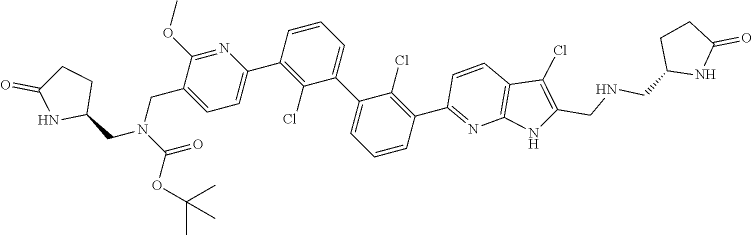 [SMILES: COc1nc(-c2cccc(-c3cccc(-c4ccc5c(Cl)c(CNC[C@@H]6CCC(=O)N6)[nH]c5n4)c3Cl)c2Cl)ccc1CN(C[C@@H]1CCC(=O)N1)C(=O)OC(C)(C)C]

tert-Butyl N-[[6-[2-chloro-3-[2-chloro-3-(3-chloro-2-formyl-1H-pyrrolo[2,3-b]pyridin-6-yl)phenyl]phenyl]-2-methoxy-3-pyridyl]methyl]-N-[[(2S)-5-oxopyrrolidin-2-yl]methyl]carbamate (28 mg, 0.04 mmol), (5S)-5-(aminomethyl)pyrrolidin-2-one (9 mg, 0.08 mmol), and acetic acid (5 mg, 0.08 mmol) were dissolved in MeOH/THF (1:1, 1 mL), then the solution was stirred at 25° C. for 2 hours. NaBH₃CN (5 mg, 0.08 mmol) was added and the resulting mixture was stirred at 25° C. for 1 hour. Reaction was subsequently diluted with water (2 mL) and extracted with EtOAc (3×2 mL). The combined organics were further washed with brine, dried over sodium sulfate, filtered, and concentrated under reduced pressure. Crude sample was purified by reverse phase HPLC to afford tert-butyl N-[[6-[2-chloro-3-[2-chloro-3-[3-chloro-2-[[[(2S)-5-oxopyrrolidin-2-yl]methylamino]methyl]-1H-pyrrolo[2,3-b]pyridin-6-yl]phenyl]phenyl]-2-methoxy-3-pyridyl]methyl]-N-[[(2S)-5-oxopyrrolidin-2-yl]methyl]carbamate (31 mg, 39.1% yield) as a white foam. MS: m/z found 834 [M+H].

### (S)-5-((((3-Chloro-6-(2,2′-dichloro-3′-(6-methoxy-5-(((((S)-5-oxopyrrolidin-2-yl)methyl)-amino)methyl)-pyridin-2-yl)-[1,1′-biphenyl]-3-yl)-1H-pyrrolo[2,3-b]pyridin-2-yl)methyl)amino)methyl)pyrrolidin-2-one

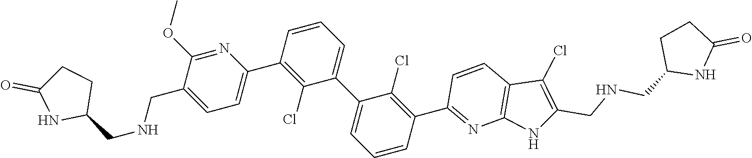 [SMILES: COc1nc(-c2cccc(-c3cccc(-c4ccc5c(Cl)c(CNC[C@@H]6CCC(=O)N6)[nH]c5n4)c3Cl)c2Cl)ccc1CNC[C@@H]1CCC(=O)N1]

tert-butyl N-[[6-[2-chloro-3-[2-chloro-3-[3-chloro-2-[[[(2S)-5-oxopyrrolidin-2-yl]methylamino]-methyl]-1H-pyrrolo[2,3-b]pyridin-6-yl]phenyl]phenyl]-2-methoxy-3-pyridyl]methyl]-N-[[(2S)-5-oxopyrrolidin-2-yl]methyl]carbamate (31 mg, 0.04 mmol) was dissolved in 2 mL DCM and 4 M HCl solution in dioxane (37 μL, 0.15 mmol) was added. Reaction was stirred at rt for 20 minutes and solvent was removed under reduced pressure. Crude sample was purified by reverse phase HPLC to afford (5S)-5-[[[6-[2-chloro-3-[2-chloro-3-[3-chloro-2-[[[(2S)-5-oxopyrrolidin-2-yl]methylamino]methyl]-1H-pyrrolo[2,3-b]pyridin-6-yl]phenyl]phenyl]-2-methoxy-3-pyridyl]methylamino]methyl]pyrrolidin-2-one (10 mg, 36.7% yield) as a white foam. MS: m/z found 732, 734 [M+H]⁺. ¹H NMR formic acid salt (400 MHz, Methanol-d₄) δ 8.42 (s, 1H), 7.98 (d, J=8.1 Hz, 1H), 7.79 (d, J=7.5 Hz, 1H), 7.62 (ddd, J=15.5, 7.7, 1.7 Hz, 2H), 7.50 (td, J=7.6, 5.9 Hz, 2H), 7.44-7.37 (m, 3H), 7.29 (d, J=7.5 Hz, 1H), 4.08-4.01 (m, 7H), 3.92 (s, 1H), 3.81 (q, J=6.9 Hz, 1H), 2.90 (h, J=7.0 Hz, 2H), 2.74 (dd, J=12.0, 4.9 Hz, 1H), 2.64 (dd, J=12.0, 7.1 Hz, 1H), 2.41-2.19 (m, 6H), 1.92-1.73 (m, 2H).

### Example 390: (5S)-5-((((6-(2,2′-Dichloro-3′-(5-(((S)-2-hydroxypropyl)amino)-5,6,7,8-tetrahydroquinolin-2-yl)-[1,1′-biphenyl]-3-yl)-2-methoxypyridin-3-yl)methyl)amino)methyl)pyrrolidin-2-one

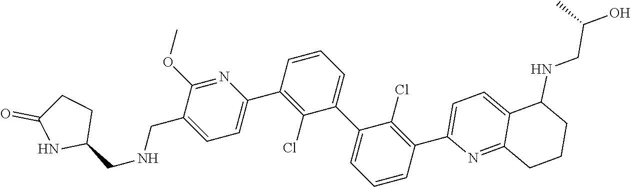 [SMILES: COc1nc(-c2cccc(-c3cccc(-c4ccc5c(n4)CCCC5NC[C@H](C)O)c3Cl)c2Cl)ccc1CNC[C@@H]1CCC(=O)N1]

### tert-Butyl (S)-((6-(2,2′-dichloro-3′-(5-oxo-5,6,7,8-tetrahydroquinolin-2-yl)-[1,1′-biphenyl]-3-yl)-2-methoxypyridin-3-yl)methyl)((5-oxopyrrolidin-2-yl)methyl)carbamate

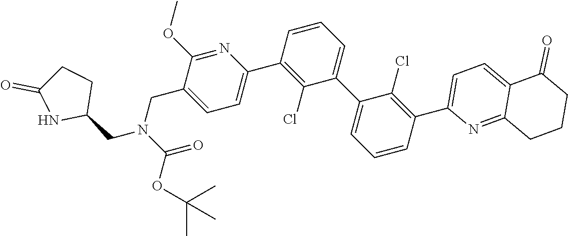 [SMILES: COc1nc(-c2cccc(-c3cccc(-c4ccc5c(n4)CCCC5=O)c3Cl)c2Cl)ccc1CN(C[C@@H]1CCC(=O)N1)C(=O)OC(C)(C)C]

Pd(PPh₃)₄ (34 mg, 0.03 mmol), potassium carbonate (61 mg, 0.44 mmol), tert-butyl N-[[6-[2-chloro-3-[2-chloro-3-(4,4,5,5-tetramethyl-1,3,2-dioxaborolan-2-yl)phenyl]phenyl]-2-methoxy-3-pyridyl]methyl]-N-[[(2S)-5-oxopyrrolidin-2-yl]methyl]carbamate (100 mg, 0.15 mmol), and 2-chloro-7,8-dihydro-6H-quinolin-5-one (35 mg, 0.19 mmol) were suspended in dioxane/water (4:1, 2 mL), then the solution was heated at 105° C. for 20 minutes. Reaction was cooled to rt, diluted with 3 ml water, and extracted with EtOAc (3×2 mL). The combined organics were concentrated under reduced pressure and crude sample was purified by normal phase SiO₂ chromatography (10-100% EtOAc/hexane) to afford tert-butyl N-[[6-[2-chloro-3-[2-chloro-3-(5-oxo-7,8-dihydro-6H-quinolin-2-yl)phenyl]-phenyl]-2-methoxy-3-pyridyl]methyl]-N-[[(2S)-5-oxopyrrolidin-2-yl]methyl]carbamate (45 mg, 43.8%) as a yellow foam. MS: m/z found 701.3, 703 [M+H]⁺.

### tert-Butyl ((6-(2,2′-dichloro-3′-(5-(((S)-2-hydroxypropyl)amino)-5,6,7,8-tetrahydroquinolin-2-yl)-[1,1′-biphenyl]-3-yl)-2-methoxypyridin-3-yl)methyl)(((S)-5-oxopyrrolidin-2-yl)methyl)carbamate (racemic)

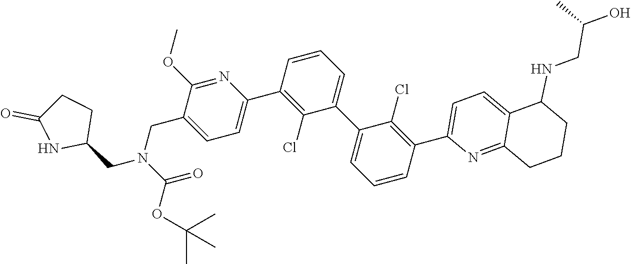 [SMILES: COc1nc(-c2cccc(-c3cccc(-c4ccc5c(n4)CCCC5NC[C@H](C)O)c3Cl)c2Cl)ccc1CN(C[C@@H]1CCC(=O)N1)C(=O)OC(C)(C)C]

tert-Butyl N-[[6-[2-chloro-3-[2-chloro-3-(5-oxo-7,8-dihydro-6H-quinolin-2-yl)-phenyl]-phenyl]-2-methoxy-3-pyridyl]-methyl]-N-[[(2S)-5-oxopyrrolidin-2-yl]methyl]carbamate (22 mg, 0.03 mmol), (2S)-1-aminopropan-2-ol carbamate (7 mg, 0.09 mmol), and tetraethoxytitanium (21 mg, 0.09 mmol) were suspended in 1 mL anhydrous THF. Reaction mixture was heated at 105° C. for 3 hours in a sealed tube. Reaction was cooled to −50° C. and NaBH₄ (2.4 mg, 0.06 mmol) was added under a nitrogen stream, then the mixture was warmed to rt over 1 hour. Reaction was subsequently added drop-wise to an ice water solution (4 mL) and the resulting slurry was filtered through CELITE®. The filtrate was extracted with EtOAc (3×5 mL) and the combined organics were further washed with brine (5 mL), dried over sodium sulfate, filtered, and concentrated under reduced pressure to afford tert-butyl N-[[6-[2-chloro-3-[2-chloro-3-[5-[[(2S)-2-hydroxypropyl]amino]-5,6,7,8-tetrahydroquinolin-2-yl]phenyl]phenyl]-2-methoxy-3-pyridyl]methyl]-N-[[(2S)-5-oxopyrrolidin-2-yl]methyl]carbamate (21 mg, crude) as a yellow foam. MS: m/z found 760.4, 762 [M+H]⁺. Material was used for the next step without further purification.

### (5S)-5-((((6-(2,2′-Dichloro-3′-(5-(((S)-2-hydroxypropyl)amino)-5,6,7,8-tetrahydroquinolin-2-yl)-[1,1′-biphenyl]-3-yl)-2-methoxypyridin-3-yl)methyl)amino)methyl)pyrrolidin-2-one (racemic)

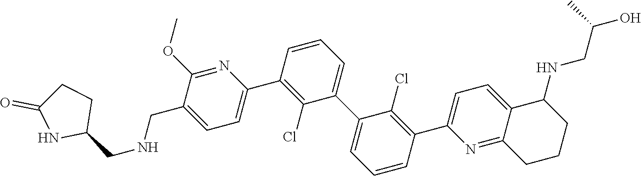 [SMILES: COc1nc(-c2cccc(-c3cccc(-c4ccc5c(n4)CCCC5NC[C@H](C)O)c3Cl)c2Cl)ccc1CNC[C@@H]1CCC(=O)N1]

tert-Butyl N-[[6-[2-chloro-3-[2-chloro-3-[5-[[(2S)-2-hydroxypropyl]amino]-5,6,7,8-tetrahydroquinolin-2-yl]phenyl]phenyl]-2-methoxy-3-pyridyl]methyl]-N-[[(2S)-5-oxopyrrolidin-2-yl]methyl]carbamate (31 mg, 0.04 mmol) was dissolved in 1 mL DCM and 4M HCl solution in dioxane (37 μL, 0.15 mmol) was added. Reaction was stirred at rt for 20 minutes and solvent was removed under reduced pressure. Crude sample was purified by reverse phase HPLC to afford (5S)-5-[[[6-[2-chloro-3-[2-chloro-3-[5-[[(2S)-2-hydroxypropyl]-amino]-5,6,7,8-tetrahydroquinolin-2-yl]-phenyl]-phenyl]-2-methoxy-3-pyridyl]methylamino]methyl]pyrrolidin-2-one (6.3 mg, 25.6% yield) as a white solid. MS: m/z found 660.5, 662 [M+H]⁺. ¹H NMR formic acid salt (400 MHz, Methanol-d₄) δ 8.53 (s, 1H), 7.96 (t, J=8.7 Hz, 1H), 7.73 (d, J=7.5 Hz, 1H), 7.63 (dd, J=7.8, 1.7 Hz, 1H), 7.55-7.45 (m, 4H), 7.42 (t, J=4.8 Hz, 1H), 7.40-7.33 (m, 1H), 7.24 (d, J=7.4 Hz, 1H), 4.13 (s, 1H), 4.01 (s, 3H), 3.94 (s, 1H), 3.84 (d, J=4.0 Hz, 3H), 3.03 (d, J=18.0 Hz, 1H), 2.93 (d, J=17.7 Hz, 1H), 2.80 (d, J=11.0 Hz, 1H), 2.74-2.63 (m, 3H), 2.37-2.23 (m, 3H), 2.07 (d, J=31.8 Hz, 3H), 1.91 (s, 1H), 1.81 (d, J=14.2 Hz, 1H), 1.21 (dd, J=6.4, 2.2 Hz, 3H).

### Example 403: (S)-7-(2,2′-Dichloro-3′-(6-methoxy-5-((((5-oxopyrrolidin-2-yl)methyl)amino)methyl)pyridin-2-yl)-[1,1′-biphenyl]-3-yl)-1,3,4,5-tetrahydro-2H-pyrido[2,3-e][1,4]diazepin-2-one

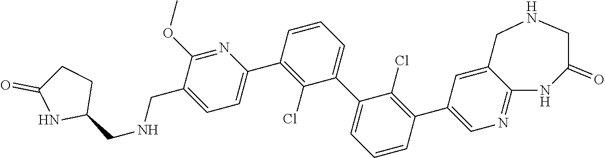 [SMILES: COc1nc(-c2cccc(-c3cccc(-c4cnc5c(c4)CNCC(=O)N5)c3Cl)c2Cl)ccc1CNC[C@@H]1CCC(=O)N1]

### tert-Butyl (S)-((6-(2,2′-dichloro-3′-(2-oxo-2,3,4,5-tetrahydro-1H-pyrido[2,3-e][1,4]diazepin-7-yl)-[1,1′-biphenyl]-3-yl)-2-methoxypyridin-3-yl)methyl)((5-oxopyrrolidin-2-yl)methyl)carbamate

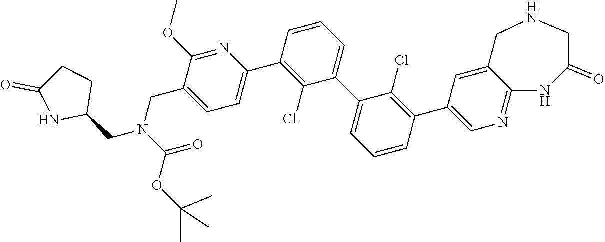 [SMILES: COc1nc(-c2cccc(-c3cccc(-c4cnc5c(c4)CNCC(=O)N5)c3Cl)c2Cl)ccc1CN(C[C@@H]1CCC(=O)N1)C(=O)OC(C)(C)C]

Pd(PPh₃)₄ (17 mg, 0.01 mmol), potassium carbonate (30 mg, 0.22 mmol), tert-butyl N-[[6-[2-chloro-3-[2-chloro-3-(4,4,5,5-tetramethyl-1,3,2-dioxaborolan-2-yl)phenyl]phenyl]-2-methoxy-3-pyridyl]methyl]-N-[[(2S)-5-oxopyrrolidin-2-yl]methyl]carbamate (50 mg, 0.07 mmol), and 7-bromo-1,3,4,5-tetrahydropyrido[2,3-e][1,4]diazepin-2-one (25 mg, 0.10 mmol) were suspended in dioxane/water (4:1, 2 mL), then the solution was heated at 105° C. for 20 minutes. Reaction was cooled to rt, diluted with 3 mL water, and extracted with EtOAc (3×2 mL). The combined organics were concentrated under reduced pressure and crude sample was purified by reverse phase HPLC to afford tert-butyl N-[[6-[2-chloro-3-[2-chloro-3-(2-oxo-1,3,4,5-tetrahydropyrido-[2,3-e][1,4]diazepin-7-yl)-phenyl]-phenyl]-2-methoxy-3-pyridyl]methyl]-N-[[(2S)-5-oxopyrrolidin-2-yl]methyl]carbamate (12 mg, 22.4% yield) as a white foam. MS: m/z found 717.3, 719.3 [M+H]⁺.

### (S)-7-(2,2′-Dichloro-3′-(6-methoxy-5-((((5-oxopyrrolidin-2-yl)methyl)amino)methyl)pyridin-2-yl)-[1,1′-biphenyl]-3-yl)-1,3,4,5-tetrahydro-2H-pyrido[2,3-e][1,4]diazepin-2-one

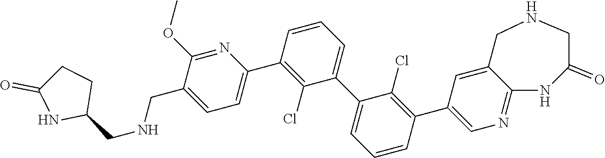 [SMILES: COc1nc(-c2cccc(-c3cccc(-c4cnc5c(c4)CNCC(=O)N5)c3Cl)c2Cl)ccc1CNC[C@@H]1CCC(=O)N1]

tert-Butyl N-[[6-[2-chloro-3-[2-chloro-3-(2-oxo-1,3,4,5-tetrahydropyrido[2,3-e][1,4]diazepin-7-yl)phenyl]phenyl]-2-methoxy-3-pyridyl]methyl]-N-[[(2S)-5-oxopyrrolidin-2-yl]methyl]carbamate (12 mg, 0.02 mmol) was dissolved in 0.5 mL DCM and 4M HCl solution in dioxane (16 μL, 0.07 mmol) was added. Reaction was stirred at rt for 20 minutes and solvent was removed under reduced pressure. Crude sample was purified by reverse phase HPLC to afford 7-[2-chloro-3-[2-chloro-3-[6-methoxy-5-[[[(2S)-5-oxopyrrolidin-2-yl]methylamino]methyl]-2-pyridyl]phenyl]phenyl]-1,3,4,5-tetrahydropyrido[2,3-e][1,4]diazepin-2-one (8.40 mg, 82.7% yield) as a white foam. MS: m/z found 617.3, 619.2 [M+H]⁺. ¹H NMR formic acid salt (400 MHz, Methanol-d₄) δ 8.33 (s, 1H), 7.82 (d, J=7.5 Hz, 1H), 7.74 (s, 1H), 7.64 (d, J=7.8 Hz, 1H), 7.53-7.44 (m, 3H), 7.38 (d, J=7.3 Hz, 2H), 7.30 (d, J=7.5 Hz, 1H), 4.13-4.03 (m, 7H), 3.96 (s, 1H), 3.81 (s, 2H), 3.00 (t, J=6.0 Hz, 2H), 2.38 (d, J=15.2 Hz, 3H), 1.94-1.80 (m, 1H).

### Example 410: (S)-8-(2,2′-Dichloro-3′-(1-methyl-2,3,4,5-tetrahydro-1H-benzo[e][1,4]diazepin-8-yl)-[1,1′-biphenyl]-3-yl)-3-((((5-oxopyrrolidin-2-yl)methyl)amino)methyl)-4H-pyrido[1,2-a]pyrimidin-4-one

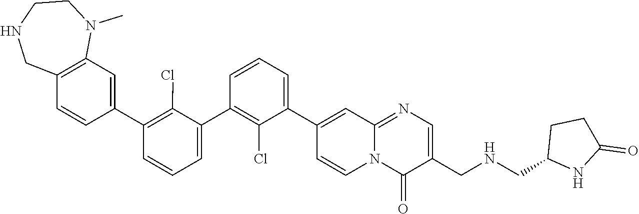 [SMILES: CN1CCNCc2ccc(-c3cccc(-c4cccc(-c5ccn6c(=O)c(CNC[C@@H]7CCC(=O)N7)cnc6c5)c4Cl)c3Cl)cc21]

### tert-Butyl 8-(2,2′-dichloro-3′-(3-formyl-4-oxo-4H-pyrido[1,2-a]pyrimidin-8-yl)-[1,1′-biphenyl]-3-yl)-1-methyl-1,2,3,5-tetrahydro-4H-benzo[e][1,4]diazepine-4-carboxylate

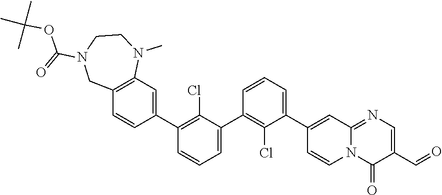 [SMILES: CN1CCN(C(=O)OC(C)(C)C)Cc2ccc(-c3cccc(-c4cccc(-c5ccn6c(=O)c(C=O)cnc6c5)c4Cl)c3Cl)cc21]

Pd(PPh₃)₄ (34 mg, 0.03 mmol), potassium carbonate (61 mg, 0.44 mmol), tert-butyl 1-methyl-8-(4,4,5,5-tetramethyl-1,3,2-dioxaborolan-2-yl)-3, 5-dihydro-2H-1,4-benzodiazepine-4-carboxylate (69 mg, 0.18 mmol), and 8-[3-(3-bromo-2-chloro-phenyl)-2-chloro-phenyl]-4-oxo-pyrido[1,2-a]pyrimidine-3-carbaldehyde (70 mg, 0.15 mmol) were suspended in dioxane/water (4:1, 2 mL), then the solution was heated at 105° C. for 20 minutes. Reaction was cooled to rt, diluted with 3 ml water, and extracted with EtOAc (3×2 mL). The combined organics were concentrated under reduced pressure and crude sample was purified by normal phase SiO₂ chromatography (5-60% EtOAc/hexane) to afford tert-butyl 8-[2-chloro-3-[2-chloro-3-(3-formyl-4-oxo-pyrido[1,2-a]pyrimidin-8-yl)phenyl]phenyl]-1-methyl-3,5-dihydro-2H-1,4-benzodiazepine-4-carboxylate (29 mg, 30% yield) as a yellow foam. MS: m/z found 655, 657 [M+H]⁺.

### tert-Butyl (S)-8-(2,2′-dichloro-3′-(4-oxo-3-((((5-oxopyrrolidin-2-yl)methyl)amino)methyl)-4H-pyrido[1,2-a]pyrimidin-8-yl)-[1,1′-biphenyl]-3-yl)-1-methyl-1,2,3,5-tetrahydro-4H-benzo[e][1,4]diazepine-4-carboxylate

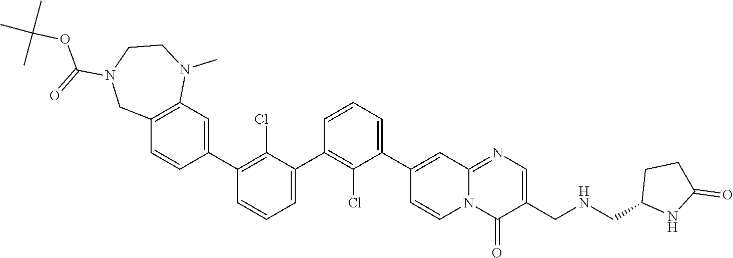 [SMILES: CN1CCN(C(=O)OC(C)(C)C)Cc2ccc(-c3cccc(-c4cccc(-c5ccn6c(=O)c(CNC[C@@H]7CCC(=O)N7)cnc6c5)c4Cl)c3Cl)cc21]

tert-Butyl 8-[2-chloro-3-[2-chloro-3-(3-formyl-4-oxo-pyrido[1,2-a]pyrimidin-8-yl)phenyl]phenyl]-1-methyl-3,5-dihydro-2H-1,4-benzodiazepine-4-carboxylate (29 mg, 0.04 mmol), (5S)-5-(aminomethyl)pyrrolidin-2-one (7.6 mg, 0.07 mmol), and acetic acid (2.7 mg, 0.04 mmol) were dissolved in MeOH/THF (1:1, 1 mL), then the solution was stirred at 25° C. for 2 hours. NaBH₃CN (5.5 mg, 0.09 mmol) was added and the resulting mixture was stirred at 25° C. for 1 hour. Reaction was subsequently diluted with water (2 mL) and extracted with DCM (3×2 mL). The combined organics were further washed with brine (3 mL), dried over sodium sulfate, filtered and concentrated under reduced pressure to afford tert-butyl 8-[2-chloro-3-[2-chloro-3-[4-oxo-3-[[[(2S)-5-oxopyrrolidin-2-yl]methylamino]methyl]pyrido[1,2-a]pyrimidin-8-yl]phenyl]phenyl]-1-methyl-3, 5-dihydro-2H-1,4-benzodiazepine-4-carboxylate (28.5 mg, crude) as yellow foam. MS: m/z found 753.4, 755.4 [M+H]⁺. Material was used for the next step without further purification.

### (S)-8-(2,2′-Dichloro-3′-(1-methyl-2,3,4,5-tetrahydro-1H-benzo[e][1,4]diazepin-8-yl)-[1,1′-biphenyl]-3-yl)-3-((((5-oxopyrrolidin-2-yl)methyl)amino)methyl)-4H-pyrido[1,2-a]pyrimidin-4-one

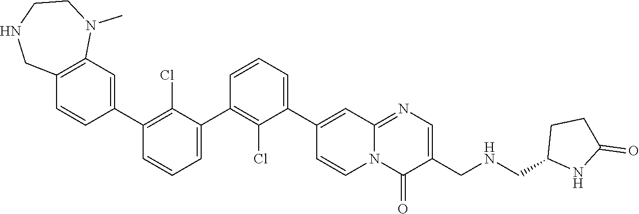 [SMILES: CN1CCNCc2ccc(-c3cccc(-c4cccc(-c5ccn6c(=O)c(CNC[C@@H]7CCC(=O)N7)cnc6c5)c4Cl)c3Cl)cc21]

tert-Butyl 8-[2-chloro-3-[2-chloro-3-[4-oxo-3-[[[(2S)-5-oxopyrrolidin-2-yl]methylamino]methyl]pyrido[1,2-a]pyrimidin-8-yl]phenyl]phenyl]-1-methyl-3, 5-dihydro-2H-1,4-benzodiazepine-4-carboxylate (28.50 mg, 0.04 mmol) was dissolved in 1 mL DCM and 4M HCl solution in dioxane (38 μL, 5.5 mg, 0.15 mmol) was added. Reaction was stirred at rt for 3 hours and solvent was removed under reduced pressure. Crude sample was purified by reverse phase HPLC to afford 8-[2-chloro-3-[2-chloro-3-(1-methyl-2,3,4,5-tetrahydro-1,4-benzodiazepin-8-yl)phenyl]phenyl]-3-[[[(2S)-5-oxopyrrolidin-2-yl]methylamino]methyl]pyrido[1,2-a]pyrimidin-4-one (8 mg, 32.4% yield) as a white foam. MS: m/z found 653, 655 [M+H]⁺. ¹H NMR formic acid salt (400 MHz, Methanol-d₄) δ 9.14 (d, J=7.4 Hz, 1H), 8.51 (s, 2H), 8.42 (s, 1H), 7.77 (d, J=1.8 Hz, 1H), 7.65-7.56 (m, 2H), 7.55-7.43 (m, 4H), 7.43-7.36 (m, 2H), 7.16 (d, J=1.6 Hz, 1H), 7.09 (dd, J=7.7, 1.6 Hz, 1H), 4.29 (s, 2H), 3.93-3.81 (m, 3H), 3.35 (d, J=8.5 Hz, 2H), 3.26 (d, J=4.9 Hz, 2H), 3.00 (s, 3H), 2.77 (qd, J=12.3, 6.2 Hz, 2H), 2.38-2.25 (m, 3H), 1.82 (dt, J=16.6, 6.6 Hz, 1H).

### Example 431: 8-(2,2′-Dichloro-3′-(6-chloro-4-methoxy-5-(((((S)-5-oxopyrrolidin-2-yl)methyl)-amino)-methyl)pyridin-2-yl)-[1,1′-biphenyl]-3-yl)-3-(((((S)-5-oxopyrrolidin-2-yl)methyl)amino)methyl)-4H-pyrido[1,2-a]pyrimidin-4-one

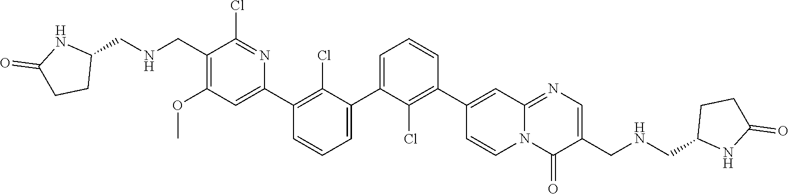 [SMILES: COc1cc(-c2cccc(-c3cccc(-c4ccn5c(=O)c(CNC[C@@H]6CCC(=O)N6)cnc5c4)c3Cl)c2Cl)nc(Cl)c1CNC[C@@H]1CCC(=O)N1]

### 8-(2,2′-Dichloro-3′-(6-chloro-5-formyl-4-methoxypyridin-2-yl)-[1,1′-biphenyl]-3-yl)-4-oxo-4H-pyrido[1,2-a]pyrimidine-3-carbaldehyde

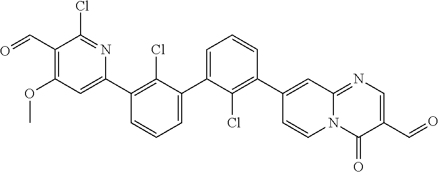 [SMILES: COc1cc(-c2cccc(-c3cccc(-c4ccn5c(=O)c(C=O)cnc5c4)c3Cl)c2Cl)nc(Cl)c1C=O]

Pd(PPh₃)₄ (44 mg, 0.04 mmol), potassium carbonate (66 mg, 0.48 mmol), 8-[2-chloro-3-[2-chloro-3-(4,4,5,5-tetramethyl-1,3,2-dioxaborolan-2-yl)phenyl]phenyl]-4-oxo-pyrido[1,2-a]pyrimidine-3-carbaldehyde (100 mg, 0.19 mmol), and 2,6-dichloro-4-methoxy-pyridine-3-carbaldehyde (51 mg, 0.25 mmol) were suspended in dioxane/water (4:1, 3 mL), then the solution was heated at 105° C. for 20 minutes. Reaction was cooled to rt, diluted with 3 ml water, and extracted with EtOAc (3×2 mL). The combined organics were concentrated under reduced pressure and crude sample was purified by normal phase SiO₂ chromatography (5-60% EtOAc/hexane) to afford 8-[2-chloro-3-[2-chloro-3-(6-chloro-5-formyl-4-methoxy-2-pyridyl)phenyl]phenyl]-4-oxo-pyrido[1,2-a]pyrimidine-3-carbaldehyde (42 mg, 38.5% yield) as a yellow solid. MS: m/z found 564.1, 566 [M+H]⁺.

### 8-(2,2′-Dichloro-3′-(6-chloro-4-methoxy-5-(((((S)-5-oxopyrrolidin-2-yl)methyl)amino)methyl)pyridin-2-yl)-[1,1′-biphenyl]-3-yl)-3-(((((S)-5-oxopyrrolidin-2-yl)methyl)amino)methyl)-4H-pyrido[1,2-a]pyrimidin-4-one

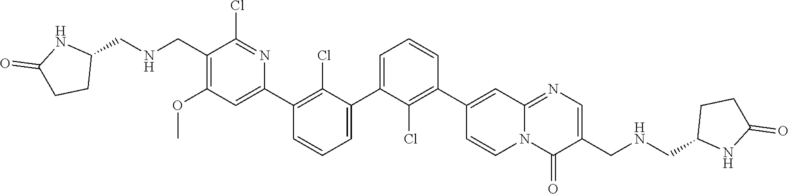 [SMILES: COc1cc(-c2cccc(-c3cccc(-c4ccn5c(=O)c(CNC[C@@H]6CCC(=O)N6)cnc5c4)c3Cl)c2Cl)nc(Cl)c1CNC[C@@H]1CCC(=O)N1]

8-[2-Chloro-3-[2-chloro-3-(6-chloro-5-formyl-4-methoxy-2-pyridyl)phenyl]phenyl]-4-oxo-pyrido[1,2-a]pyrimidine-3-carbaldehyde (41.70 mg, 0.07 mmol), (5S)-5-(aminomethyl)pyrrolidin-2-one (25 mg, 0.22 mmol), and acetic acid (9 mg, 0.15 mmol) were dissolved in MeOH/THF (1:1, 2 mL), then the solution was stirred at 25° C. for 2 hours. NaBH₃CN (9 mg, 0.15 mmol) was added and the resulting mixture was stirred at 25° C. for 1 hour. Reaction was subsequently diluted with water (3 mL) and extracted with EtOAc (3×2 mL). The combined organics were further washed with brine, dried over sodium sulfate, filtered, and concentrated under reduced pressure. Crude sample was purified by reverse phase HPLC to afford 8-[2-chloro-3-[2-chloro-3-[6-chloro-4-methoxy-5-[[[(2S)-5-oxopyrrolidin-2-yl]methylamino]methyl]-2-pyridyl]phenyl]phenyl]-3-[[[(2S)-5-oxopyrrolidin-2-yl]methylamino]methyl]pyrido[1,2-a]pyrimidin-4-one (28.6 mg, 51% yield) as a white foam. MS: m/z found 762 [M+H]. ¹H NMR formic acid salt (400 MHz, Methanol-d₄) δ 9.16 (d, J=7.2 Hz, 1H), 8.46 (s, 2H), 7.80 (s, 1H), 7.67-7.42 (m, 7H), 7.32 (s, 1H), 4.06 (s, 4H), 4.01 (d, J=1.7 Hz, 3H), 3.94 (s, 1H), 3.89-3.80 (m, 1H), 2.93 (d, J=8.7 Hz, 2H), 2.75 (t, J=7.5 Hz, 2H), 2.43-2.20 (m, 6H), 1.92-1.72 (m, 2H).

### Example 432: (5S)-5-(((3-(2,2′-Dichloro-3′-(6-methoxy-5-(((((S)-5-oxopyrrolidin-2-yl)-methyl)-amino)methyl)-pyridin-2-yl)-[1,1′-biphenyl]-3-yl)-5,6,7,8-tetrahydroquinolin-8-yl)amino)-methyl)-pyrrolidin-2-one

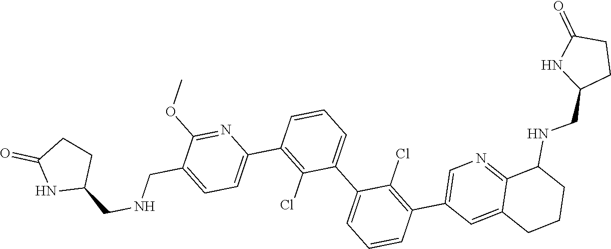 [SMILES: COc1nc(-c2cccc(-c3cccc(-c4cnc5c(c4)CCCC5NC[C@@H]4CCC(=O)N4)c3Cl)c2Cl)ccc1CNC[C@@H]1CCC(=O)N1]

### tert-Butyl (S)-((6-(2,2′-dichloro-3′-(8-oxo-5,6,7,8-tetrahydroquinolin-3-yl)-[1,1′-biphenyl]-3-yl)-2-methoxypyridin-3-yl)methyl)((5-oxopyrrolidin-2-yl)methyl)carbamate

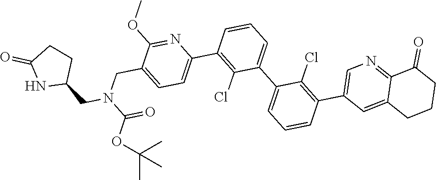 [SMILES: COc1nc(-c2cccc(-c3cccc(-c4cnc5c(c4)CCCC5=O)c3Cl)c2Cl)ccc1CN(C[C@@H]1CCC(=O)N1)C(=O)OC(C)(C)C]

Pd(PPh₃)₄ (24 mg, 0.02 mmol), potassium carbonate (42 mg, 0.31 mmol), tert-butyl N-[[6-[2-chloro-3-[2-chloro-3-(4,4,5,5-tetramethyl-1,3,2-dioxaborolan-2-yl)phenyl]phenyl]-2-methoxy-3-pyridyl]methyl]-N-[[(2S)-5-oxopyrrolidin-2-yl]methyl]carbamate (100 mg, 0.10 mmol), and 3-bromo-6,7-dihydro-5H-quinolin-8-one (30 mg, 0.13 mmol) were suspended in dioxane/water (4:1, 2 mL), then the solution was heated at 105° C. for 20 minutes. Reaction was cooled to rt, diluted with 3 mL water, and extracted with EtOAc (3×2 mL). The combined organics were concentrated under reduced pressure and crude sample was purified by normal phase SiO₂ chromatography (0-6% MeOH/DCM) to afford tert-butyl N-[[6-[2-chloro-3-[2-chloro-3-(8-oxo-6,7-dihydro-5H-quinolin-3-yl)phenyl]-phenyl]-2-methoxy-3-pyridyl]methyl]-N-[[(2S)-5-oxopyrrolidin-2-yl]methyl]carbamate (60 mg, 83% yield) as a yellow foam. MS: m/z found 701.3, 703 [M+H]⁺.

### tert-Butyl ((6-(2,2′-dichloro-3′-(8-((((S)-5-oxopyrrolidin-2-yl)methyl)amino)-5,6,7,8-tetrahydroquinolin-3-yl)-[1,1′-biphenyl]-3-yl)-2-methoxypyridin-3-yl)methyl)(((S)-5-oxopyrrolidin-2-yl)methyl)carbamate

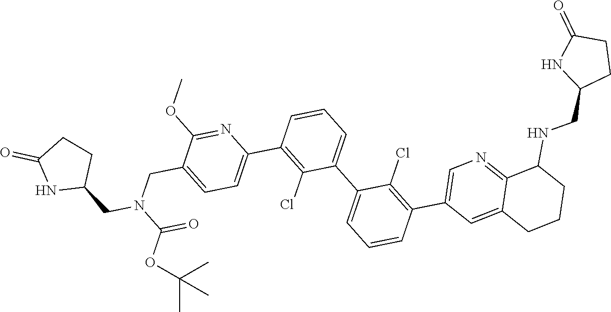 [SMILES: COc1nc(-c2cccc(-c3cccc(-c4cnc5c(c4)CCCC5NC[C@@H]4CCC(=O)N4)c3Cl)c2Cl)ccc1CN(C[C@@H]1CCC(=O)N1)C(=O)OC(C)(C)C]

tert-butyl N-[[6-[2-chloro-3-[2-chloro-3-(8-oxo-6,7-dihydro-5H-quinolin-3-yl)-phenyl]phenyl]-2-methoxy-3-pyridyl]methyl]-N-[[(2S)-5-oxopyrrolidin-2-yl]methyl]carbamate (60 mg, 0.08 mmol), (5S)-5-(aminomethyl)pyrrolidin-2-one (19 mg, 0.17 mmol), and tetraethoxytitanium (58 mg, 0.25 mmol) were suspended in 3 mL anhydrous THF. Reaction mixture was heated at 105° C. for 3 hours in a sealed tube. Reaction was cooled to −50° C. and NaBH₄ (6.5 mg, 0.17 mmol) was added portion-wise under a nitrogen stream, then the solution was warmed to rt over 1 hour. Reaction was subsequently added drop-wise to an ice water solution (15 mL) and the resulting slurry was filtered through CELITE®. The filtrate was extracted with EtOAc (3×5 mL) and the combined organics were further washed with brine (5 mL), dried over sodium sulfate, filtered, and concentrated under reduced pressure to afford tert-butyl N-[[6-[2-chloro-3-[2-chloro-3-[8-[[(2S)-5-oxopyrrolidin-2-yl]methylamino]-5,6,7,8-tetrahydroquinolin-3-yl]phenyl]phenyl]-2-methoxy-3-pyridyl]methyl]-N-[[(2S)-5-oxopyrrolidin-2-yl]methyl]carbamate (52 mg, crude) as a yellow foam. MS: m/z found 799.4, 801.4 [M+H]⁺. Material was used for the next step without further purification.

### (5S)-5-(((3-(2,2′-Dichloro-3′-(6-methoxy-5-(((((S)-5-oxopyrrolidin-2-yl)methyl)amino)methyl)pyridin-2-yl)-[1,1′-biphenyl]-3-yl)-5,6,7,8-tetrahydroquinolin-8-yl)amino)methyl)pyrrolidin-2-one

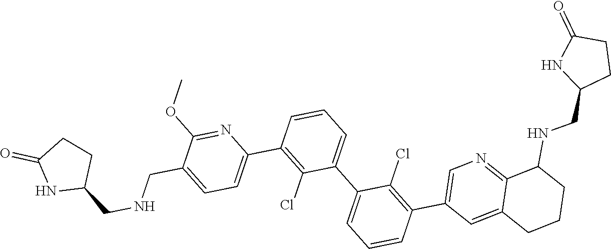 [SMILES: COc1nc(-c2cccc(-c3cccc(-c4cnc5c(c4)CCCC5NC[C@@H]4CCC(=O)N4)c3Cl)c2Cl)ccc1CNC[C@@H]1CCC(=O)N1]

tert-Butyl N-[[6-[2-chloro-3-[2-chloro-3-[8-[[(2S)-5-oxopyrrolidin-2-yl]methylamino]-5,6,7,8-tetrahydroquinolin-3-yl]phenyl]phenyl]-2-methoxy-3-pyridyl]methyl]-N-[[(2S)-5-oxopyrrolidin-2-yl]methyl]carbamate (52 mg, 0.07 mmol) was dissolved in 2 mL DCM and 4M HCl solution in dioxane (65 μL, 0.26 mmol) was added. Reaction was stirred at rt for 20 minutes and solvent was removed under reduced pressure. Crude sample was purified by reverse phase HPLC to afford (5S)-5-[[[6-[2-chloro-3-[2-chloro-3-[8-[[(2S)-5-oxopyrrolidin-2-yl]methylamino]-5,6,7,8-tetrahydroquinolin-3-yl]phenyl]phenyl]-2-methoxy-3-pyridyl]methylamino]methyl]pyrrolidin-2-one (10 mg, 23% yield) as a light yellow solid. MS: m/z found 699.4, 701 [M+H]⁺. ¹H NMR formic acid salt (400 MHz, Methanol-d₄) δ 8.54 (d, J=1.9 Hz, 1H), 8.47 (s, 1H), 7.77 (d, J=7.4 Hz, 1H), 7.71 (d, J=2.1 Hz, 1H), 7.64 (dd, J=7.7, 1.7 Hz, 1H), 7.55-7.43 (m, 3H), 7.38 (ddd, J=13.8, 7.4, 1.8 Hz, 2H), 7.26 (d, J=7.4 Hz, 1H), 4.16 (s, 1H), 4.03 (s, 3H), 4.00-3.86 (m, 4H), 3.12-2.88 (m, 4H), 2.83 (t, J=6.4 Hz, 2H), 2.43-2.27 (m, 7H), 2.11 (d, J=12.1 Hz, 1H), 1.97-1.77 (m, 4H).

### Example 203: 8-[2-chloro-3-[2-Chloro-3-[8-chloro-5-methoxy-6-[[[(2S)-5-oxopyrrolidin-2-yl]methylamino] methyl]-2-quinolyl]phenyl]phenyl]-3-[[[(2S)-5-oxopyrrolidin-2-yl]methylamino]methyl] pyrido[1,2-a]pyrimidin-4-one

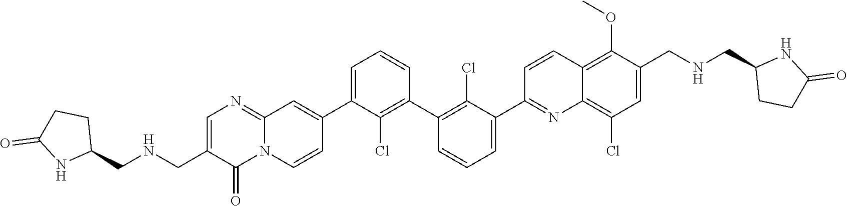 [SMILES: COc1c(CNC[C@@H]2CCC(=O)N2)cc(Cl)c2nc(-c3cccc(-c4cccc(-c5ccn6c(=O)c(CNC[C@@H]7CCC(=O)N7)cnc6c5)c4Cl)c3Cl)ccc12]

### 4-Amino-5-chloro-2-methoxybenzoic acid

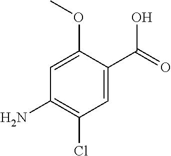

To a mixture methyl 4-acetamido-5-chloro-2-methoxybenzoate (100 g, 388 mmol) in EtOH/water (2:1, 600 mL) was added sodium hydroxide (62.1 g, 1.55 mol). The mixture was stirred at 80° C. for 12 h. The mixture was diluted with water and treated with concentrated hydrochloric acid. The resulting precipitate was filtered, washed with water and dried to give 4-amino-5-chloro-2-methoxybenzoic acid as a white solid (120 g, crude).

### Methyl 8-chloro-5-methoxyquinoline-6-carboxylate

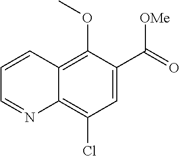 [SMILES: COC(=O)c1cc(Cl)c2ncccc2c1OC]

To a mixture of 4-amino-5-chloro-2-methoxybenzoic acid (20 g, 99.2 mmol) in concentrated sulfuric acid/water (5:1 (v v), 96 mL) was added glycerol (41.1 g, 446 mmol) and sodium 3-nitrobenzenesulfonate (44.7 g, 198 mmol). The mixture was stirred at 100° C. for 3 h and 140° C. for 1h. The mixture was cooled to 60° C., diluted with MeOH (40 mL) and continued stirring at 60° C. for 10 h. The solvent was evaporated, residue poured into ice-water mixture and basified with aqueous ammonium hydroxide. The mixture was diluted with ethyl acetate (2 L), filtered to remove charred material, organic layer separated, dried and evaporated. The residue was purified by silica gel chromatography (0-100%0 EtOAc/petroleum ether) to afford methyl-8-chloro-5-methoxyquinoline-6-carboxylate as a yellow solid (4 g, 16% yield). ¹H NMR (400 MHz, DMSO-d): 9.15 (dd, J=1.6 Hz, J=4.4 Hz, 1H), 8.70 (dd, J=1.6 Hz, J=8.4 Hz 1H), 8.16 (s, 1H), 7.78 (dd, J=4 Hz, J=8.4 Hz, 1H), 4.00 (m, 3H), 3.93 (m, 3H).

### Methyl 2,8-dichloro-5-methoxyquinoline-6-carboxylate

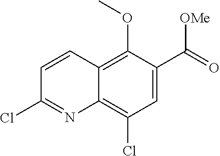 [SMILES: COC(=O)c1cc(Cl)c2nc(Cl)ccc2c1OC]

To a mixture of methyl-2-bromo-5-methoxyquinoline-6-carboxylate (0.6 g, 2.03 mmol) in DMF (10 mL) was added n-chlorosuccinimide (1.08 g, 8.1 mmol) and glacial acetic acid (0.01 g, 0.2 mmol). The mixture was stirred at 80° C. for 12 h. The reaction mixture was diluted with water (8 mL), and the resulting solid was collected by filtration to afford methyl-2,8-dichloro-5-methoxyquinoline-6-carboxylate (0.75 g). ¹H NMR (400 MHz, DMSO-d₆): δ 8.70 (d, J=8.8 Hz, 1H), 8.20 (s, 1H), 7.81 (d, J=8.4 Hz, 1H), 3.99 (s, 3H), 3.93 (s, 3H)

### (2,8-Dichloro-5-methoxyquinolin-6-yl)methanol

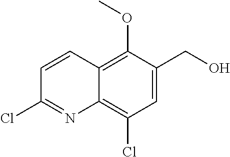 [SMILES: COc1c(CO)cc(Cl)c2nc(Cl)ccc12]

To a mixture of methyl-2,8-dichloro-5-methoxyquinoline-6-carboxylate (0.4 g, 1.4 mmol) in THE (5 mL) was added lithium aluminum hydride (0.06 g, 1.68 mmol). The mixture was stirred at rt for 1 h. The reaction was quenched with water (5 mL) at 0° C., and then dried over anhydrous sodium sulfate, filtered and concentrated under reduced pressure to give a residue. The residue was purified by silica gel chromatography (0-100% EtOAc/petroleum ether) to afford 2,8-dichloro-5-methoxyquinolin-6-yl-methanol as a yellow solid (0.22 g). ¹H NMR (400 MHz, DMSO-d₆): δ 8.53 (d, J=8.8 Hz 1H), 8.02 (s, 1H), 7.69 (d, J=8.8 Hz, 1H), 5.47 (br, 1H), 4.69 (s, 2H), 3.88 (s, 3H).

### 2,8-Dichloro-5-methoxyquinoline-6-carbaldehyde

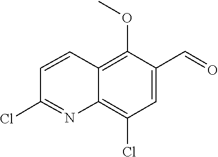 [SMILES: COc1c(C=O)cc(Cl)c2nc(Cl)ccc12]

To a mixture of 2,8-dichloro-5-methoxyquinolin-6-yl-methanol (0.2 g, 0.77 mmol) in DCM (2 mL) was added manganese dioxide (0.67 g, 7.75 mmol). The mixture was stirred at rt for 3 h. The reaction mixture was filtered and concentrated under reduced pressure to give 2,8-dichloro-5-methoxyquinoline-6-carbaldehyde as a yellow solid (0.2 g, crude). ¹H NMR (400 MHz, CDCl₃): δ 10.43 (s, 1H), 8.46 (d, J=8.8 Hz, 1H), 8.17 (s, 1H), 7.50 (d, J=8.8 Hz, 1H), 4.09 (s, 3H).

### 8-(2,2′-Dichloro-3′-(8-chloro-6-formyl-5-methoxyquinolin-2-yl)-[1,1′-biphenyl]-3-yl)-4-oxo-4H-pyrido[1,2-a]pyrimidine-3-carbaldehyde

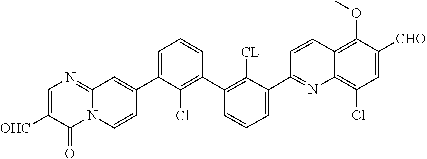

To a mixture of 2,8-dichloro-5-methoxyquinoline-6-carbaldehyde (0.1 g, 0.39 mmol) in dioxane/water (3:1, 8 mL) was added 8-(2,2′-dichloro-3′-(4,4,5,5-tetramethyl-1,3,2-dioxaborolan-2-yl)-[1,1′-biphenyl]-3-yl)-4-oxo-4H-pyrido[1,2-a]pyrimidine-3-carbaldehyde (0.26 g, 0.51 mmol), [1,1′-bis(di-tert-butylphosphino)ferrocene]dichloropalladium(II) (0.03 g, 0.04 mmol) and potassium phosphate (0.25 g, 1.17 mmol). The mixture was stirred at 110° C. for 0.5 h. The reaction mixture was quenched with water (30 mL), and then extracted with EtOAc (3×30 ml). The combined organic layers were washed with brine (20 mL), dried over sodium sulfate, filtered and concentrated. The residue was purified by prep-TLC (SiO₂, Petroleum ether/Ethyl acetate=2/1) to afford 8-(2,2′-dichloro-3′-(8-chloro-6-formyl-5-methoxyquinolin-2-yl)-[1,1′-biphenyl]-3-yl)-4-oxo-4H-pyrido[1,2-a]pyrimidine-3-carbaldehyde as a yellow solid (0.07 g, 29% yield, m/z: 614 [M]⁺, 615.9 [M+2]⁺ observed.

### 8-(2,2′-Dichloro-3′-(8-chloro-5-methoxy-6-(((((S)-5-oxopyrrolidin-2-yl)methyl)amino) methyl)quinolin-2-yl)-[1,1′-biphenyl]-3-yl)-3-(((((S)-5-oxopyrrolidin-2-yl)methyl)amino) methyl)-4H-pyrido[1,2-a]pyrimidin-4-one

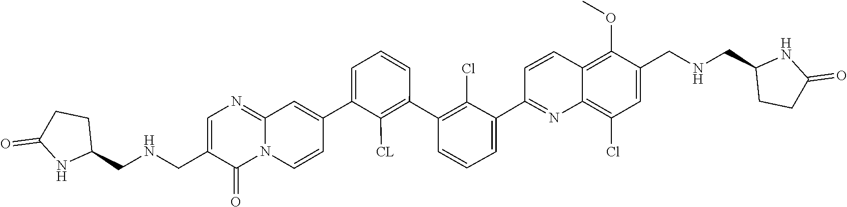

To a mixture of 8-(2,2′-dichloro-3′-(8-chloro-6-formyl-5-methoxyquinolin-2-yl)-[1,1′-biphenyl]-3-yl)-4-oxo-4H-pyrido[1,2-a]pyrimidine-3-carbaldehyde (0.07 g, 0.11 mmol) in MeOH/DCM (1:1, 4 mL) was added (S)-5-(aminomethyl)pyrrolidin-2-one, HCl (0.1 g, 0.68 mmol) and sodium acetate (0.06 g, 0.68 mmol). The mixture was stirred at rt for 2 h. Sodium cyanoborohydride (0.04 g, 0.68 mmol) was added and the mixture was stirred at rt for 1 h. The reaction mixture was filtered and concentrated under reduced pressure. The residue was purified by reverse phase HPLC to afford 8-(2,2′-dichloro-3′-(8-chloro-5-methoxy-6-(((((S)-5-oxopyrrolidin-2-yl)methyl)amino)methyl)quinolin-2-yl)-[1,1′-biphenyl]-3-yl)-3-(((((S)-5-oxopyrrolidin-2-yl)methyl)amino)methyl)-4H-pyrido[1,2-a]pyrimidin-4-one as a yellow solid (9 mg, 9% yield, LCMS: 810.2 [M]⁺, 812.2 [M+2]⁺ observed. ¹H NMR (400 MHz, Methanol-d₄): δ 9.20 (d, J=7.6 Hz, 1H), 8.67 (d, J=8.8 Hz, 1H), 8.50 (s, 1H), 8.07 (s, 1H), 7.96 (d, J=8.8 Hz, 1H), 7.86 (d, J=1.2 Hz, 1H), 7.80 (dd, J=1.6 Hz, J=7.6 Hz, 1H), 7.67-7.64 (m, 5H), 7.54 (dd, J=1.6 Hz, J=7.6 Hz, 1H), 4.16 (m, 4H), 4.05-4.01 (m, 4H), 3.92-3.91 (m, 1H), 3.07-3.02 (m, 2H), 2.85-2.83 (m, 2H), 2.42-2.33 (m, 6H), 1.91-1.85 (m, 2H).

### Example 176: 8-(2,2′-Dichloro-3′-(5-methoxy-6-(((((S)-5-oxopyrrolidin-2-yl)methyl)amino)methyl) quinolin-2-yl)-[1,1′-biphenyl]-3-yl)-3-(((((S)-5-oxopyrrolidin-2-yl)methyl)amino)methyl)-4H-pyrido[1,2-a]pyrimidin-4-one

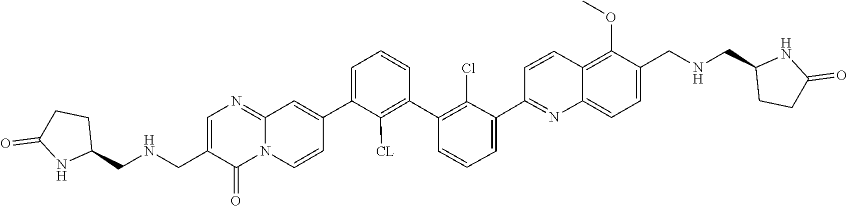

### 8-Chloro-5-methoxyquinoline-6-carboxylic acid

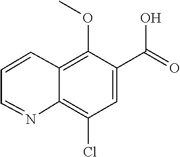 [SMILES: COc1c(C(=O)O)cc(Cl)c2ncccc12]

To a mixture of methyl-8-chloro-5-methoxyquinoline-6-carboxylate (7 g, 27.8 mmol) in MeOH/water (7:2, 90 mL) was added lithium hydroxide monohydrate (3.50 g, 83.4 mmol). The mixture was stirred at 15° C. for 15 h. The reaction mixture was concentrated under reduced pressure. The reaction mixture was quenched with water (30 mL) and extracted with EtOAc (3×30 ml). The aqueous phase was treated with concentrated hydrochloric acid (pH=3) and filtered to give 8-chloro-5-methoxyquinoline-6-carboxylic acid as a white solid (7 g, crude). ¹H NMR (400 MHz, DMSO-d₆): δ 9.07 (dd, J=1.6 Hz, J=4 Hz, 1H), 8.64 (dd, J=2 Hz, J=8 Hz 1H), 8.10 (s, 1H), 7.66 (dd, J=4 Hz, J=8.4 Hz, 1H), 3.95 (s, 3H).

5-Methoxyquinoline-6-carboxylic acid

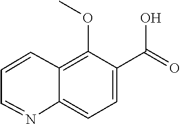

To a mixture of 8-chloro-5-methoxyquinoline-6-carboxylic acid (1 g, 4.21 mmol) in EtOH (80 mL) was added hydrazine hydrate (1.05 g, 21 mmol) and 10% Pd/C (1 g). The mixture was stirred at 80° C. for 14 h. The reaction mixture was filtered and concentrated under reduced pressure to give 5-methoxyquinoline-6-carboxylic acid as a red solid (5 g, crude). ¹H NMR (400 MHz, DMSO-d₆): δ 8.86 (dd, J=1.6 Hz, J=4.4 Hz, 1H), 8.50 (d, J=8.4 Hz, 1H), 7.80 (d, J=8.8 Hz, 1H), 7.66 (d, J=8.8 Hz, 1H), 7.53 (dd, J=4 Hz, J=8.4 Hz, 1H), 4.00 (s, 3H).

### Methyl 5-methoxyquinoline-6-carboxylate

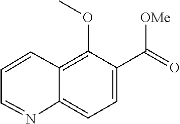 [SMILES: COC(=O)c1ccc2ncccc2c1OC]

To a mixture of 5-methoxyquinoline-6-carboxylic acid (5 g, 24.6 mmol) in MeOH (20 mL) was added thionyl chloride (4.39 g, 36.9 mmol). The mixture was stirred at 70° C. for 4 h. The reaction mixture was concentrated under reduced pressure. The mixture was diluted with water (30 mL) and extracted with EtOAc (3×30 ml). The combined organic layers were washed with brine (20 mL), dried over sodium sulfate, filtered and concentrated under reduced pressure. The residue was purified by silica gel chromatography (0-100% EtOAc/petroleum ether) to afford methyl 5-methoxyquinoline-6-carboxylate as a yellow solid (5 g, 93% yield).

### 5-Methoxy-6-(methoxycarbonyl)quinoline 1-oxide

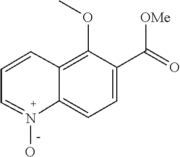

To a mixture of methyl-5-methoxyquinoline-6-carboxylate (5 g, 23 mmol) in DCM (5 mL) was added 3-chloroperbenzoic acid (5.96 g, 34.5 mmol). The mixture was stirred at 15° C. for 12 h. The reaction mixture was diluted with saturated sodium sulfite solution (50 mL) and extracted with DCM (3×50 ml). The combined organic layers were washed with brine (30 mL), dried over sodium sulfate, filtered and concentrated under reduced pressure. The residue was purified by silica gel chromatography (0-100% EtOAc/petroleum ether to 0-100% Ethyl acetate/MeOH) to get 5-methoxy-6-(methoxycarbonyl)quinoline 1-oxide as a white solid (3 g, 55% yield). ¹H NMR (400 MHz, DMSO-d₆): δ 8.71 (dd, J=0.8 Hz, J=6 Hz, 1H), 8.34 (d, J=9.2 Hz, 1H), 8.11 (d, J=8.8 Hz, 1H), 8.04 (d, J=9.6 Hz, 1H), 7.60-7.56 (m, 1H), 4.02 (s, 3H), 3.95 (m, 3H)

### Methyl 2-bromo-5-methoxyquinoline-6-carboxylate

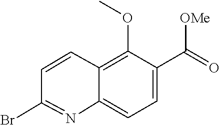 [SMILES: COC(=O)c1ccc2nc(Br)ccc2c1OC]

To a mixture of 5-methoxy-6-(methoxycarbonyl)quinoline-1-oxide (3 g, 12.86 mmol,) in CHCl₃ (10 mL) was added phosphorus oxybromide (11.1 g, 38.6 mmol). The mixture was stirred at 50° C. for 4 h. The reaction mixture was added to water (90 mL) and was extracted with DCM (3×90 ml). The combined organic layers were washed with brine (60 mL), dried over sodium sulfate, filtered and concentrated under reduced pressure. The residue was purified by silica gel chromatography (0-100% EtOAc/petroleum ether to 0-100% EtOAc/MEOH) to afford methyl-2-bromo-5-methoxyquinoline-6-carboxylate as a white solid (3 g, 79% yield). ¹H NMR (400 MHz, DMSO-d₆): δ 8.58 (dd, J=0.8 Hz, J=8.8 Hz, 1H), 8.11 (d, J=8.8 Hz, 1H), 7.88-7.84 (m, 2H), 4.05 (s, 3H), 3.98 (m, 3H).

### (2-Bromo-5-methoxyquinolin-6-yl)methanol

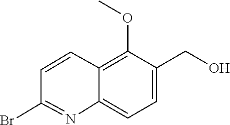

To a mixture of methyl 2-bromo-5-methoxyquinoline-6-carboxylate (1.3 g, 4.39 mmol) in THE (5 mL) was added lithium aluminum hydride (0.2 g, 5.27 mmol). The mixture was stirred at 20° C. for 1 h. The reaction mixture was diluted with water (5 mL) at 0° C., dried over sodium sulfate, filtered and concentrated under reduced pressure. The residue was purified by silica gel chromatography (0-100% EtOAc/petroleum ether) to afford (2-bromo-5-methoxyquinolin-6-yl)methanol as a yellow oil (0.5 g, 42% yield). ¹H NMR (400 MHz, DMSO-d₆): δ 8.33 (dd, J=1.2 Hz, J=8.8 Hz, 1H), 7.89 (d, J=8.8 Hz, 1H), 7.74 (d, J=8.8 Hz, 1H), 7.67 (d, J=8.8 Hz, 1H), 5.34 (d, J=6 Hz, 1H), 4.67 (d, J=6 Hz, 2H), 3.85 (s, 3H).

### 2-Bromo-5-methoxyquinoline-6-carbaldehyde

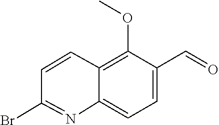

To a mixture of 2-bromo-5-methoxyquinolin-6-yl-methanol (0.5 g, 1.86 mmol) in CHCl₃ (5 mL) was added manganese dioxide (1.62 g, 18.7 mmol). The mixture was stirred at rt for 18 h. The reaction mixture was filtered, and the resulting yellow solid was collected by filtration. The solid was dried to afford 2-bromo-5-methoxyquinoline-6-carbaldehyde as a white solid (0.35 g, 70% yield). ¹H NMR (400 MHz, DMSO-d₆): δ 10.48 (s, 1H), 8.58 (d, J=8.8 Hz, 1H), 8.06 (d, J=8.8 Hz, 1H), 7.85 (m, 2H), 4.15 (s, 3H).

### 8-(2,2′-Dichloro-3′-(6-formyl-5-methoxyquinolin-2-yl)-[1,1′-biphenyl]-3-yl)-4-oxo-4H-pyrido[1,2-a]pyrimidine-3-carbaldehyde

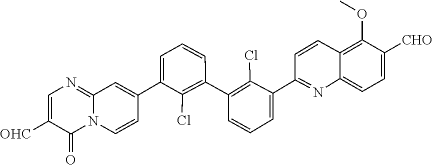 [SMILES: COc1c(C=O)ccc2nc(-c3cccc(-c4cccc(-c5ccn6c(=O)c(C=O)cnc6c5)c4Cl)c3Cl)ccc12]

To a mixture of 2-bromo-5-methoxyquinoline-6-carbaldehyde (0.35 g, 1.32 mmol) in dioxane/water (3:1, 8 mL) was added 8-(2,2′-dichloro-3′-(1,5-dimethyl-2,4-dioxa-3-borabicyclo[3.1.0]hexan-3-yl)-[1,1′-biphenyl]-3-yl)-4-oxo-4H-pyrido[1,2-a]pyrimidine-3-carbaldehyde (0.75 g, 1.45 mmol), [1,1′-bis(di-tert-butylphosphino)ferrocene]dichloropalladium(II) (0.09 g, 0.13 mmol) and potassium phosphate (0.84 g, 3.95 mmol). The mixture was stirred at 110° C. for 0.5 h. The reaction mixture was diluted with water (30 mL) and extracted with EtOAc (3×40 ml). The combined organic layers were washed with brine (20 mL), dried over sodium sulfate, filtered and concentrated under reduced pressure. The residue was purified by silica gel chromatography (0-100% EtOAc/petroleum ether) to afford 8-(2,2′-dichloro-3′-(6-formyl-5-methoxyquinolin-2-yl)-[1,1′-biphenyl]-3-yl)-4-oxo-4H-pyrido[1,2-a]pyrimidine-3-carbaldehyde as a yellow solid (0.3 g, 39% yield, m/z: 580 [M+H]+ observed.

### 8-(2,2′-Dichloro-3′-(5-methoxy-6-(((((S)-5-oxopyrrolidin-2-yl)methyl)amino)methyl) quinolin-2-yl)-[1,1′-biphenyl]-3-yl)-3-(((((S)-5-oxopyrrolidin-2-yl)methyl)amino)methyl)-4H-pyrido[1,2-a]pyrimidin-4-one

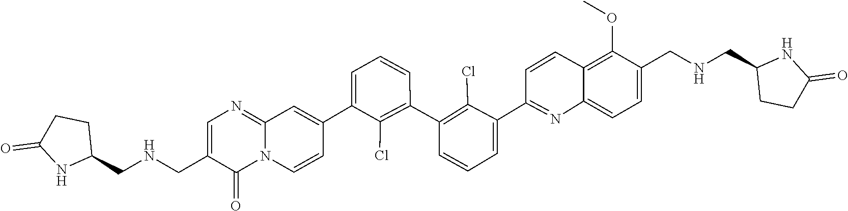 [SMILES: COc1c(CNC[C@@H]2CCC(=O)N2)ccc2nc(-c3cccc(-c4cccc(-c5ccn6c(=O)c(CNC[C@@H]7CCC(=O)N7)cnc6c5)c4Cl)c3Cl)ccc12]

To a mixture of 8-(2,2′-dichloro-3′-(6-formyl-5-methoxyquinolin-2-yl)-[1,1′-biphenyl]-3-yl)-4-oxo-4H-pyrido[1,2-a]pyrimidine-3-carbaldehyde (0.26 g, 0.45 mmol) in MeOH/DCM (1:1, 4 mL) was added (S)-5-(aminomethyl)pyrrolidin-2-one, HCl (0.4 g, 2.69 mmol) and sodium acetate (0.22 g, 2.69 mmol). After stirring the mixture at rt for 2 h, sodium cyanoborohydride (0.17 g, 2.69 mmol) was added. The mixture was stirred at rt for 1 h. The reaction mixture was filtered and concentrated under reduced pressure. The residue was purified by reverse phase HPLC to afford 8-(2,2′-dichloro-3′-(5-methoxy-6-(((((S)-5-oxopyrrolidin-2-yl)methyl)amino) methyl)quinolin-2-yl)-[1,1′-biphenyl]-3-yl)-3-(((((S)-5-oxopyrrolidin-2-yl)methyl)amino) methyl)-4H-pyrido[1,2-a]pyrimidin-4-one as a yellow solid. (26 mg, 7% yield, m/z: 776.2 [M+H]⁺ observed. ¹H NMR (400 MHz, Methanol-d₄): δ 9.15 (d, J=7.6 Hz, 1H), 8.67 (d, J=8.8 Hz, 1H), 8.43 (s, 1H), 7.94-7.92 (m, 2H), 7.84-7.79 (m, 2H), 7.71 (dd, J=2 Hz, J=7.6 Hz, 1H), 7.67-7.61 (m, 3H), 7.58-7.54 (m, 3H), 4.09 (m, 2H), 4.04 (m, 3H), 3.90-3.84 (m, 4H), 2.79-2.69 (m, 4H), 2.38-2.29 (m, 6H), 1.87-1.80 (m, 2H).

### Example 194: (S)-5-((((8-Chloro-2-(2,2′-dichloro-3′-(6-methoxy-5-(((((S)-5-oxopyrrolidin-2-yl)methyl)amino)methyl)pyridin-2-yl)-[1,1′-biphenyl]-3-yl)-5-methoxyquinolin-6-yl)methyl)amino)methyl)pyrrolidin-2-one

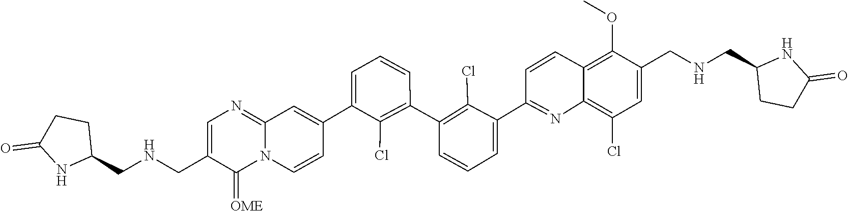

### 8-Chloro-2-(2,2′-dichloro-3′-(5-formyl-6-methoxypyridin-2-yl)-[1,1′-biphenyl]-3-yl)-5-methoxyquinoline-6-carbaldehyde

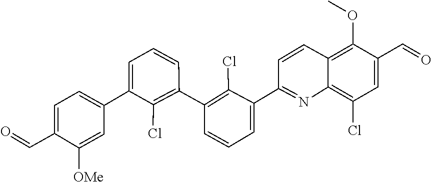 [SMILES: COc1cc(-c2cccc(-c3cccc(-c4ccc5c(OC)c(C=O)cc(Cl)c5n4)c3Cl)c2Cl)ccc1C=O]

To a mixture of 2,8-dichloro-5-methoxyquinoline-6-carbaldehyde (0.08 g, 0.31 mmol) in THF/water (1:1, 2 mL) was added 6-(2,2′-dichloro-3′-(4,4,5,5-tetramethyl-1,3,2-dioxaborolan-2-yl)-[1,1′-biphenyl]-3-yl)-2-methoxynicotinaldehyde (0.15 g, 0.31 mmol), potassium carbonate (0.13 g, 0.94 mmol) and 1,1′-bis(diphenylphosphino)ferrocene-palladium(ii) dichloride dichloromethane complex (0.03 g, 0.03 mmol). The mixture was stirred at 80° C. for 1 h. The reaction mixture was diluted with water (30 mL) and extracted with EtOAc (3×30 mL). The combined organic layers were washed with brine (20 mL), dried over sodium sulfate, filtered and concentrated under reduced pressure. The residue was purified by prep-TLC (SiO₂, Petroleum ether/Ethyl acetate=2/1) to get 8-chloro-2-(2,2′-dichloro-3′-(5-formyl-6-methoxypyridin-2-yl)-[1,1′-biphenyl]-3-yl)-5-methoxyquinoline-6-carbaldehyde as colorless oil (85 mg, 47% yield, LCMS: 577.2 [M]⁺,579.1 [M+2] observed.

### (S)-5-((((8-Chloro-2-(2,2′-dichloro-3′-(6-methoxy-5-(((((S)-5-oxopyrrolidin-2-yl)methyl)amino)methyl)pyridin-2-yl)-[1,1′-biphenyl]-3-yl)-5-methoxyquinolin-6-yl)methyl)amino)methyl)pyrrolidin-2-one

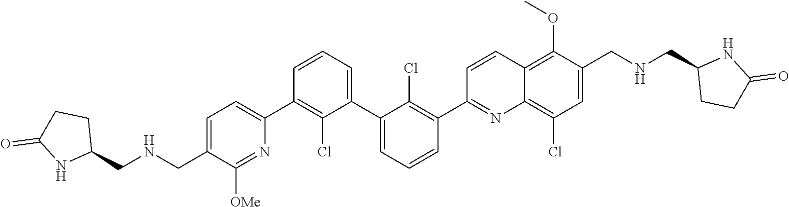 [SMILES: COc1nc(-c2cccc(-c3cccc(-c4ccc5c(OC)c(CNC[C@@H]6CCC(=O)N6)cc(Cl)c5n4)c3Cl)c2Cl)ccc1CNC[C@@H]1CCC(=O)N1]

To a mixture of 8-chloro-2-(2,2′-dichloro-3′-(5-formyl-6-methoxypyridin-2-yl)-[1,1′-biphenyl]-3-yl)-5-methoxyquinoline-6-carbaldehyde (0.08 g, 0.14 mmol) in MeOH/DCM (1:1, 2 mL) was added (S)-5-(aminomethyl)pyrrolidin-2-one, HCl (0.13 g, 0.83 mmol,) and sodium acetate (0.07 g, 0.83 mmol). After stirring the mixture at rt for 1 h, sodium cyanoborohydride (0.03 g, 0.55 mmol) was added. The mixture was stirred at 25° C. for 0.5 h. The reaction mixture was filtered and concentrated under reduced pressure. The residue was purified by reverse phase HPLC to afford (S)-5-((((8-chloro-2-(2,2′-dichloro-3′-(6-methoxy-5-(((((S)-5-oxopyrrolidin-2-yl)methyl) amino)methyl)pyridin-2-yl)-[1,1′-biphenyl]-3-yl)-5-methoxyquinolin-6-yl)methyl)amino) methyl)pyrrolidin-2-one as a white solid (34 mg, 31% yield, MS: 773.1 [M]⁺, 775.1[M+2]⁺, observed. ¹H NMR (400 MHz, Methanol-d₄): δ 8.62 (d, J=8.8 Hz, 1H), 8.04 (s, 1H), 7.92 (d, J=8.8 Hz, 1H), 7.76-7.72 (m, 2H), 7.64 (dd, J=1.6 Hz, J=7.6 Hz, 1H), 7.58 (t, J=8 Hz, 1H), 7.50-7.49 (m, 2H), 7.41 (dd, J=1.6 Hz, J=7.6 Hz, 1H), 7.25 (d, J=7.2 Hz, 1H), 4.04 (m, 2H), 4.02 (s, 3H), 4.01 (s, 3H), 3.87-3.79 (m, 4H), 2.75-2.66 (m, 4H), 2.36-2.26 (m, 6H), 1.88-1.77 (m, 2H).

### Example 164: 8-(2,2′-Dichloro-3′-(6-methoxy-5-(((((S)-5-oxopyrrolidin-2-yl)methyl)amino)methyl)pyridin-2-yl)-[1,1′-biphenyl]-3-yl)-2-methyl-3-(((((S)-5-oxopyrrolidin-2-yl)methyl)amino)methyl)-4H-pyrido[1,2-a]pyrimidin-4-one

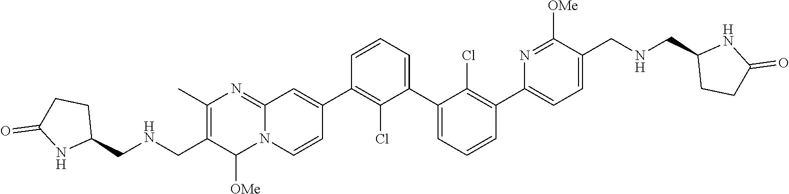 [SMILES: COc1nc(-c2cccc(-c3cccc(C4=CC5=NC(C)=C(CNC[C@@H]6CCC(=O)N6)C(OC)N5C=C4)c3Cl)c2Cl)ccc1CNC[C@@H]1CCC(=O)N1]

### Ethyl 8-bromo-2-methyl-4-oxo-4H-pyrido[1,2-a]pyrimidine-3-carboxylate

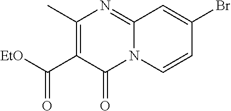 [SMILES: CCOC(=O)c1c(C)nc2cc(Br)ccn2c1=O]

A mixture of 4-bromopyridin-2-amine (5 g, 29 mmol) and diethyl 2-(1-ethoxyethylidene) propanedioate (6.65 g, 29 mmol) was stirred at 120° C. for 12 h under N₂. The mixture was purified by silica gel chromatography (0-40% EtOAc/petroleum ether) to afford ethyl 8-bromo-2-methyl-4-oxo-4H-pyrido[1,2-a]pyrimidine-3-carboxylate as a yellow solid (3.3 g, 36% yield). ¹H NMR (400 MHz, CDCl₃): δ8.78 (d, J=7.6 Hz, 1H), 7.99 (d, J=1.6 Hz, 1H), 7.54 (dd, J=7.6 Hz, J=2 Hz, 1H), 4.28 (q, J=7.2 Hz, 2H), 2.40 (s, 3H), 1.27 (t, J=7.2 Hz, 3H).

### 8-Bromo-3-(hydroxymethyl)-2-methyl-4H-pyrido[1,2-a]pyrimidin-4-one

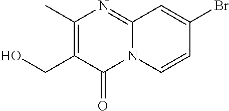

To a mixture of ethyl 8-bromo-2-methyl-4-oxo-4H-pyrido[1,2-a]pyrimidine-3-carboxylate (3.1 g, 10 mmol) in DCM (60 mL) at −10° C., bis(2-methylpropyl)alumanylium hydride (1 M in toluene, 25 mL, 25 mmol) was added. After stirring the reaction mixture at rt for 4 h, water (25 mL) was added and continued stirring at rt for 0.5 h. The mixture was filtered. The filtrate was extracted with DCM (2×100 mL). The combined organic phase was dried with sodium sulfate, filtered and concentrated. The residue was purified by silica gel chromatography (0-90% EtOAc/petroleum ether) to afford 8-bromo-3-(hydroxymethyl)-2-methyl-4H-pyrido[1,2-a]pyrimidin-4-one as a yellow solid (1.7 g, 63% yield). ¹H NMR (400 MHz, DMSO-d₆): δ. 8.73 (d, J=6.8 Hz, 1H), 7.87 (d, J=2.4 Hz, 1H), 7.40 (dd, J=7.6 Hz, J=2.4 Hz, 1H), 4.78 (t, J=5.2 Hz, 1H), 4.49 (d, J=7.2 Hz, 2H), 2.44 (s, 3H).

### 8-Bromo-2-methyl-4-oxo-4H-pyrido[1,2-a]pyrimidine-3-carbaldehyde

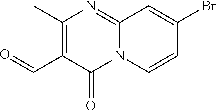

A mixture of 8-bromo-3-(hydroxymethyl)-2-methyl-4H-pyrido[1,2-a]pyrimidin-4-one (1.5 g, 5.5 mmol) and manganese(IV) oxide (4.85 g, 55 mmol) in CHCl₃ (50 mL) was stirred at 60° C. for 12 h under N₂. The mixture was filtered. The filtrate was concentrated to afford 8-bromo-2-methyl-4-oxo-4H-pyrido[1,2-a]pyrimidine-3-carbaldehyde as a crude yellow solid (1.2 g, 80% yield). ¹H NMR (400 MHz, DMSO-d₆): δ10.34 (s, 1H), 8.93 (d, J=7.2 Hz, 1H), 8.09 (d, J=1.6 Hz, 1H), 7.68 (dd, J=7.2 Hz, J=2 Hz, 1H), 2.65 (s, 3H). 8-(3-Bromo-2-chlorophenyl)-2-methyl-4-oxo-4H-pyrido[1,2-a]pyrimidine-3-carbaldehyde

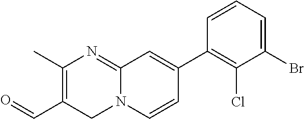 [SMILES: CC1=C(C=O)CN2C=CC(c3cccc(Br)c3Cl)=CC2=N1]

To a mixture of 8-bromo-2-methyl-4-oxo-4H-pyrido[1,2-a]pyrimidine-3-carbaldehyde (1.2 g, 4.5 mmol) and 2-(3-bromo-2-chlorophenyl)-4,4,5,5-tetramethyl-1,3,2-dioxaborolane (1.43 g, 4.5 mmol) in THF/water mixture (5:1, 30 mL) was added 1,1′-bis(diphenylphosphino)ferrocene-palladium(ii) dichloride dichloromethane complex (0.37 g, 0.45 mmol) and potassium carbonate (1.86 g, 13 mmol). The mixture was stirred at 80° C. for 3 h under N₂. The mixture was filtered. The filter cake was wash with water (30 mL) and EtOAc/petroleum ether mixture (1:2, 100 mL). The solid obtained was dried in vacuo to afford 8-(3-bromo-2-chlorophenyl)-2-methyl-4-oxo-4H-pyrido[1,2-a]pyrimidine-3-carbaldehyde as a brown solid (1 g, 53% yield, m/z: 379[M]⁺, 377[M-2]⁺ observed).

### 8-(2,2′-Dichloro-3′-(5-formyl-6-methoxypyridin-2-yl)-[1,1′-biphenyl]-3-yl)-2-methyl-4-oxo-4H-pyrido[1,2-a]pyrimidine-3-carbaldehyde

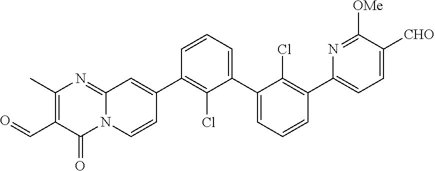 [SMILES: COc1nc(-c2cccc(-c3cccc(-c4ccn5c(=O)c(C=O)c(C)nc5c4)c3Cl)c2Cl)ccc1C=O]

To a mixture of 8-(3-bromo-2-chlorophenyl)-2-methyl-4-oxo-4H-pyrido[1,2-a]pyrimidine-3-carbaldehyde (0.7 g, 1.85 mmol) and 6-(2-chloro-3-(4,4,5,5-tetramethyl-1,3,2-dioxaborolan-2-yl)phenyl)-2-methoxynicotinaldehyde (0.7 g, 1.85 mmol) in dioxane/water mixture (4:1, 10 mL) was added di-terbutyl(cyclopentyl)phosphane dichloropalladium iron (0.12 g, 0.18 mmol) and potassium phosphate (1.18 g, 5.56 mmol). The mixture was stirred at 110° C. for 1 h under N₂. The mixture was concentrated and the residue was purified by silica gel chromatography (0-55% EtOAc/petroleum ether) to afford 8-(2,2′-dichloro-3′-(5-formyl-6-methoxypyridin-2-yl)-[1,1′-biphenyl]-3-yl)-2-methyl-4-oxo-4H-pyrido[1,2-a]pyrimidine-3-carbaldehyde as a brown solid (0.22 g, 18% yield, m/z: 544 [M+H]⁺ observed).

### 8-(2,2′-Dichloro-3′-(6-methoxy-5-(((((S)-5-oxopyrrolidin-2-yl)methyl)amino)methyl)pyridin-2-yl)-[1,1′-biphenyl]-3-yl)-2-methyl-3-(((((S)-5-oxopyrrolidin-2-yl)methyl)amino)methyl)-4H-pyrido[1,2-a]pyrimidin-4-one

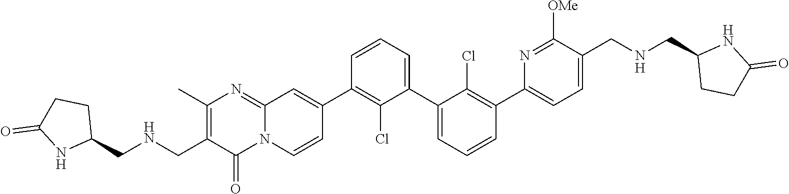 [SMILES: COc1nc(-c2cccc(-c3cccc(-c4ccn5c(=O)c(CNC[C@@H]6CCC(=O)N6)c(C)nc5c4)c3Cl)c2Cl)ccc1CNC[C@@H]1CCC(=O)N1]

To a mixture of 8-(2,2′-dichloro-3′-(5-formyl-6-methoxypyridin-2-yl)-[1,1′-biphenyl]-3-yl)-2-methyl-4-oxo-4H-pyrido[1,2-a]pyrimidine-3-carbaldehyde (0.2 g, 0.36 mmol) and (S)-5-(aminomethyl)pyrrolidin-2-one, HCl (0.33 g, 2.2 mmol) in DCM (5 mL) was added sodium acetate (0.24 g, 2.94 mmol) and sodium triacetyloxyboranuide (0.47 g, 2.2 mmol). The mixture was stirred at rt for 2.5 h under N₂. The mixture was filtered and the filtrate was concentrated. The residue was purified by reverse phase HPLC to afford 8-(2,2′-dichloro-3′-(6-methoxy-5-(((((S)-5-oxopyrrolidin-2-yl)methyl)amino)methyl)pyridin-2-yl)-[1,1′-biphenyl]-3-yl)-2-methyl-3-(((((S)-5-oxopyrrolidin-2-yl)methyl)amino)methyl)-4H-pyrido[1,2-a]pyrimidin-4-one as a light yellow solid (61 mg, 22% yield, m/z: 740 [M+H]⁺ observed). ¹H NMR (400 MHz, DMSO-d₆): δ8.94 (d, J=7.2 Hz, 1H), 7.81 (d, J=7.6 Hz, 1H), 7.68-7.64 (m, 6H), 7.56-7.54 (m, 2H), 7.54-7.41 (m, 2H), 7.26 (d, J=7.6 Hz, 1H), 3.90 (s, 3H), 3.77 (s, 2H), 3.70 (s, 2H), 3.64-3.58 (m, 2H), 2.54-2.50 (m, 7H) (overlapped with solvent peak), 2.13-2.03 (m, 6H), 1.70-1.63 (m, 2H).

### Example 173: (S)-1-(5-Chloro-2-(2-cyanoethoxy)-4-((E)-2-(2,2′-dichloro-3′-(4-oxo-3-(((((S)-5-oxopyrrolidin-2-yl)methyl)amino)methyl)-4H-pyrido[1,2-a]pyrimidin-8-yl)-[1,1′-biphenyl]-3-yl)vinyl)benzyl)pyrrolidine-3-carboxylic acid

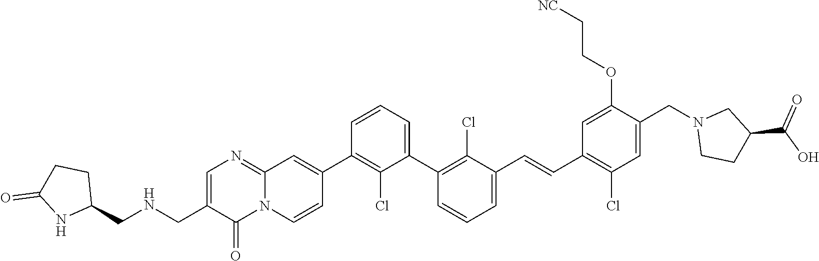 [SMILES: N#CCCOc1cc(/C=C/c2cccc(-c3cccc(-c4ccn5c(=O)c(CNC[C@@H]6CCC(=O)N6)cnc5c4)c3Cl)c2Cl)c(Cl)cc1CN1CC[C@H](C(=O)O)C1]

### 4-Bromo-5-chloro-2-hydroxybenzaldehyde

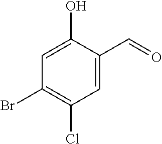 [SMILES: O=Cc1cc(Cl)c(Br)cc1O]

To a mixture of 5-bromo-4-chloro-2-(hydroxymethyl)phenol (83 g, 0.35 mol) in THE (700 mL) was added manganese dioxide (190 g, 2.19 mol) in one portion under N₂. The mixture was stirred at 60° C. for 10 h. The mixture was filtered and the filtrate was concentrated. The residue was purified by silica gel chromatography (0-10% THF/petroleum ether) to afford 4-bromo-5-chloro-2-hydroxy-benzaldehyde as a light-yellow solid (52 g, 63% yield). ¹H NMR (400 MHz, DMSO-d₆): δ 11.30 (s, 1H), 10.17 (s, 1H), 7.70 (s, 1H), 7.36 (s, 1H).

### 5-Chloro-2-hydroxy-4-vinylbenzaldehyde

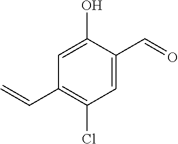

To a mixture of 4-bromo-5-chloro-2-hydroxybenzaldehyde (43 g, 0.18 mol) and potassium trifluoro(vinyl)borate (49 g, 0.36 mol) in dioxane (600 mL) and water (90 mL) was added 1,1′-bis(diphenylphosphino)ferrocene-palladium(II)dichloride dichloromethane complex (7.46 g, 9.13 mmol) and potassium carbonate (50.5 g, 0.36 mol) in one portion under N₂. The mixture was stirred at 80° C. for 13 h. The mixture was combined with another batch at 15 g scale. The mixture was concentrated. The residue was suspended in water (300 mL) and brine (200 mL). The mixture was extracted with ethyl acetate/THF mixture (1:1, 400 mL). The organic phase was dried with anhydrous sodium sulfate, filtered and concentrated. The residue was purified by silica gel chromatography (0-10% 30% THE in ethyl acetate/petroleum ether) to afford 5-chloro-2-hydroxy-4-vinylbenzaldehyde as a light-yellow solid (37 g, 82% yield). ¹H NMR (400 MHz, CDCl₃): δ 10.74 (s, 1H), 9.75 (s, 1H), 7.48 (s, 1H), 7.11 (s, 1H), 7.03-6.96 (m, 1H), 5.82 (d, J=17.2 Hz, 1H), 5.50 (d, J=10.8 Hz, 1H).

### (E)-4-(3-Bromo-2-chlorostyryl)-5-chloro-2-hydroxybenzaldehyde

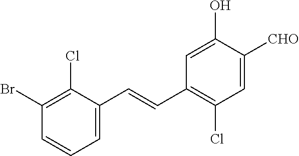 [SMILES: O=Cc1cc(Cl)c(/C=C/c2cccc(Br)c2Cl)cc1O]

To a mixture of 5-chloro-2-hydroxy-4-vinyl-benzaldehyde (20 g, 109.6 mmol) and 1-bromo-2-chloro-3-iodo-benzene (41.2 g, 131.4 mmol) in dioxane (400 mL) was added tetrabutylammonium chloride (3.04 g, 10.96 mmol), tris-o-tolylphosphane (0.66 g, 2.2 mmol), palladium diacetate (0.50 g, 2.2 mmol) and N-ethyl-N-isopropylpropan-2-amine (38 mL, 220 mmol) in one portion under N₂. The mixture was stirred at 120° C. for 16 h. The mixture was concentrated in vacuo. To the residue was added water (400 mL) and brine solution (300 mL). The mixture was extracted with ethyl acetate/THF mixture (1:1, 2×400 mL). The organic phase was dried with anhydrous sodium sulfate, filtered and concentrated. The residue was purified by silica gel chromatography (0-30% (30% THE in ethyl acetate)/petroleum ether) to afford 4-[(E)-2-(3-bromo-2-chloro-phenyl)vinyl]-5-chloro-2-hydroxy-benzaldehyde as a yellow solid (15 g, 35% yield). ¹H NMR (400 MHz, DMSO-d₆): δ 11.06 (s, 1H), 10.28 (s, 1H), 7.98 (d, J=7.6 Hz, J=1.2 Hz, 1H), 7.86 (d, J=8 Hz, J=1.2 Hz, 1H), 7.75 (s, 1H), 7.63-7.59 (m, 1H), 7.51 (s, 2H), 7.42 (t, J=8 Hz, 1H).

### (E)-4-(3-Bromo-2-chlorostyryl)-2-(2-bromoethoxy)-5-chlorobenzaldehyde

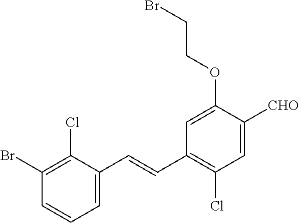 [SMILES: O=Cc1cc(Cl)c(/C=C/c2cccc(Br)c2Cl)cc1OCCBr]

To a mixture of (E)-4-(3-bromo-2-chlorostyryl)-5-chloro-2-hydroxybenzaldehyde (2 g, 5.38 mmol) and 1,2-dibromoethane (2 mL, 27 mmol) in DMF (10 mL) was added potassium carbonate (2.23 g, 16 mmol) in one portion under N₂ atmosphere. The mixture was stirred at 50° C. for 12 h. To the mixture was added water (10 mL) and brine (100 mL). The mixture was extracted with ethyl acetate/THF mixture (1:1, 100 mL). The organic phase was concentrated to afford (E)-4-(3-bromo-2-chlorostyryl)-2-(2-bromoethoxy)-5-chlorobenzaldehyde as a yellow solid (2 g, 77% yield). ¹H NMR (400 MHz, DMSO-d₆): δ 10.37 (s, 1H), 7.90 (d, J=8 Hz, 1H), 7.82-7.73 (m, 4H), 7.45-7.41 (m, 1H), 7.37 (t, J=8 Hz, 1H), 4.68 (t, J=5.2 Hz, 2H), 3.93 (t, J=5.2 Hz, 2H).

### (E)-2-(4-(3-Bromo-2-chlorostyryl)-2-(2-bromoethoxy)-5-chlorophenyl)-1,3-dioxolane

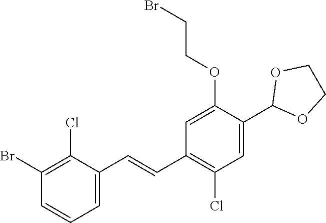 [SMILES: Clc1cc(C2OCCO2)c(OCCBr)cc1/C=C/c1cccc(Br)c1Cl]

To a mixture of (E)-4-(3-bromo-2-chlorostyryl)-2-(2-bromoethoxy)-5-chlorobenzaldehyde (1.7 g, 3.55 mmol) and ethylene glycol (0.6 mL, 10.7 mmol) in toluene (30 mL) was added p-toluenesulfonic acid (0.06 g, 0.35 mmol) in one portion. The mixture was stirred at 120° C. for 1 h under N₂ atmosphere. The mixture was added to the saturated aqueous sodium bicarbonate solution (80 mL) and water (20 mL). The mixture was extracted with ethyl acetate/THF mixture (1:1, 100 mL). The organic phase was dried with anhydrous sodium sulfate, filtered and concentrated. The residue was purified by silica gel chromatography (0-15% 30% THF in ethyl acetate/petroleum ether) to afford (E)-2-(4-(3-bromo-2-chlorostyryl)-2-(2-bromoethoxy)-5-chlorophenyl)-1,3-dioxolane as a yellow solid (1.1 g, 59% yield). ¹H NMR (400 MHz, DMSO-d₆): δ 7.87 (d, J=6.8 Hz, 1H), 7.78 (d, J=8 Hz, 1H), 7.65-7.61 (m, 1H), 7.52 (s, 1H), 7.47 (s, 1H), 7.41-7.33 (m, 2H), 6.05 (s, 1H), 4.52 (t, J=5.2 Hz, 2H), 4.11-3.93 (m, 4H), 3.85 (t, J=5.2 Hz, 2H).

### (E)-3-(5-(3-Bromo-2-chlorostyryl)-4-chloro-2-(1,3-dioxolan-2-yl)phenoxy)propanenitrile

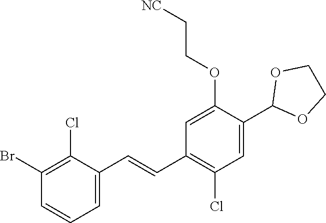 [SMILES: N#CCCOc1cc(/C=C/c2cccc(Br)c2Cl)c(Cl)cc1C1OCCO1]

To a mixture of(E)-2-(4-(3-bromo-2-chlorostyryl)-2-(2-bromoethoxy)-5-chlorophenyl)-1,3-dioxolane (1.1 g, 2.1 mmol) in DMF (6 mL) was added potassium cyanide (0.27 g, 4.2 mmol) in one portion. The mixture was stirred at rt for 12 h and then at 40° C. for 12 h. To the mixture was added water (50 mL) and brine (50 mL). The mixture was extracted with ethyl acetate/THF mixture (1:1, 200 mL). The organic phase was dried with anhydrous sodium sulfate, filtered and concentrated. The residue was purified by normal phase SiO₂ chromatography (0-15% (30% THF in ethyl acetate)/petroleum ether) to afford (E)-3-(5-(3-bromo-2-chlorostyryl)-4-chloro-2-(1,3-dioxolan-2-yl)phenoxy)propanenitrile as a yellow solid (0.3 g, 30% yield). ¹H NMR (400 MHz, DMSO-d₆): δ 7.92 (d, J=7.2 Hz, 1H), 7.83 (d, J=8 Hz, 1H), 7.71-7.67 (m, 1H), 7.59 (s, 1H), 7.54 (s, 1H), 7.47-7.41 (m, 2H), 6.08 (s, 1H), 4.45 (t, J=6 Hz, 2H), 4.15-3.99 (m, 4H), 3.12 (t, J=6 Hz, 2H).

### (E)-3-(5-(3-Bromo-2-chlorostyryl)-4-chloro-2-formylphenoxy)propanenitrile

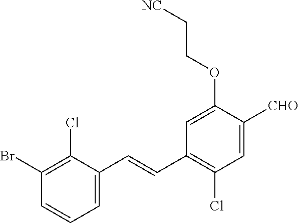 [SMILES: N#CCCOc1cc(/C=C/c2cccc(Br)c2Cl)c(Cl)cc1C=O]

To a mixture of (E)-3-(5-(3-bromo-2-chlorostyryl)-4-chloro-2-(1,3-dioxolan-2-yl)phenoxy) propanenitrile (0.26 g, 0.55 mmol) in THF (2 mL) was added aqueous 6 N HCl solution (3 mL) in one portion. The mixture was stirred at rt for 1 h. To the mixture was added water (50 mL) and brine (5 mL). The mixture was extracted with ethyl acetate/THF mixture (1:1, 60 mL). The organic phase was dried with anhydrous sodium sulfate, filtered and concentrated to afford (E)-3-(5-(3-bromo-2-chlorostyryl)-4-chloro-2-formylphenoxy)propanenitrile as a yellow solid (0.23 g, 97% yield). ¹H NMR (400 MHz, DMSO-d₆): δ 10.39 (s, 1H), 7.92 (d, J=7.2 Hz, 1H), 7.86-7.78 (m, 4H), 7.50-7.40 (m, 2H), 4.59 (t, J=6 Hz, 2H), 3.19 (t, J=6 Hz, 2H).

### (E)-3-(4-Chloro-5-(2-chloro-3-(4,4,5,5-tetramethyl-1,3,2-dioxaborolan-2-yl)styryl)-2-formylphenoxy)propanenitrile

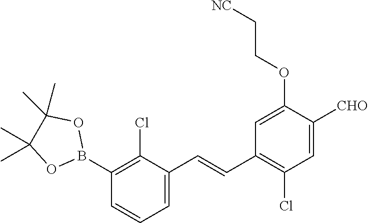 [SMILES: CC1(C)OB(c2cccc(/C=C/c3cc(OCCC#N)c(C=O)cc3Cl)c2Cl)OC1(C)C]

To a mixture of (E)-3-(5-(3-bromo-2-chlorostyryl)-4-chloro-2-formylphenoxy)propanenitrile (0.23 g, 0.54 mmol) and bis(pinacolato)diboron (0.41 g, 1.62 mmol) in 1,4-dioxane (5 mL) was added 1,1′-bis(diphenylphosphino)ferrocene-palladium(II)dichloride dichloromethane complex (0.02 g, 0.03 mmol) and potassium acetate (0.27 g, 2.71 mmol) in one portion. The mixture was stirred at 110° C. for 0.5 h under N₂ atmosphere. The mixture was concentrated, and the residue was purified by normal phase SiO₂ chromatography (0-20% 30% THE in ethyl acetate/petroleum ether) to afford (E)-3-(4-chloro-5-(2-chloro-3-(4,4,5,5-tetramethyl-1,3,2-dioxaborolan-2-yl)styryl)-2-formylphenoxy)propanenitrile as a yellow gum (0.22 g, 65% yield). ¹H NMR (400 MHz, DMSO-d₆): δ 10.35 (s, 1H), 7.92 (s, 1H), 7.80-7.71 (m, 3H), 7.61 (d, J=6.4 Hz, 1H), 7.44-7.38 (m, 2H), 4.54 (t, J=6 Hz, 2H), 3.15-3.12 (m, 2H), 1.17 (s, 12H).

### (S,E)-tert-Butyl ((8-(2,2′-dichloro-3′-(2-chloro-5-(2-cyanoethoxy)-4-formylstyryl)-[1,1′-biphenyl]-3-yl)-4-oxo-4H-pyrido[1,2-a]pyrimidin-3-yl)methyl)((5-oxopyrrolidin-2-yl)methyl)carbamate

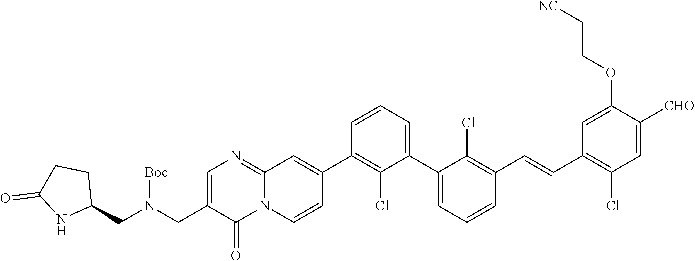 [SMILES: CC(C)(C)OC(=O)N(Cc1cnc2cc(-c3cccc(-c4cccc(/C=C/c5cc(OCCC#N)c(C=O)cc5Cl)c4Cl)c3Cl)ccn2c1=O)C[C@@H]1CCC(=O)N1]

To a mixture of (E)-3-(4-chloro-5-(2-chloro-3-(4,4,5,5-tetramethyl-1,3,2-dioxaborolan-2-yl)styryl)-2-formylphenoxy)propanenitrile (0.2 g, 0.42 mmol) and (S)-tert-butyl ((8-(3-bromo-2-chlorophenyl)-4-oxo-4H-pyrido[1,2-a]pyrimidin-3-yl)methyl)((5-oxopyrrolidin-2-yl)methyl)carbamate (0.21 g, 0.38 mmol) in 1,4-dioxane/water mixture (10:1, 2.2 mL) was added [1,1′-Bis(di-tert-butylphosphino)ferrocene]dichloropalladium(II) (0.03 g, 0.04 mmol) and potassium phosphate (0.27 g, 1.27 mmol) in one portion. The mixture was stirred at 110° C. for 0.5 h under N₂ atmosphere. To the mixture was added water (5 mL) and brine (5 mL). The mixture was extracted with ethyl acetate/THF mixture (1:1, 20 mL). The organic phase was dried with anhydrous sodium sulfate, filtered and concentrated. The residue was purified by normal phase SiO₂ chromatography (30-100% 30% THE in ethyl acetate/petroleum ether to 5% MeOH/ethyl acetate) to afford (S,E)-tert-butyl ((8-(2,2′-dichloro-3′-(2-chloro-5-(2-cyanoethoxy)-4-formylstyryl)-[1,1′-biphenyl]-3-yl)-4-oxo-4H-pyrido[1,2-a]pyrimidin-3-yl)methyl)((5-oxopyrrolidin-2-yl)methyl)carbamate as a yellow solid (0.2 g, 25% yield, 45% purity, m/z: 826 [M+H]⁺ observed).

### (S)-1-(4-((E)-2-(3′-(3-(((tert-Butoxycarbonyl)(((S)-5-oxopyrrolidin-2-yl)methyl)amino)methyl)-4-oxo-4H-pyrido[1,2-a]pyrimidin-8-yl)-2,2′-dichloro-[1,1′-biphenyl]-3-yl)vinyl)-5-chloro-2-(2-cyanoethoxy)benzyl)pyrrolidine-3-carboxylic acid

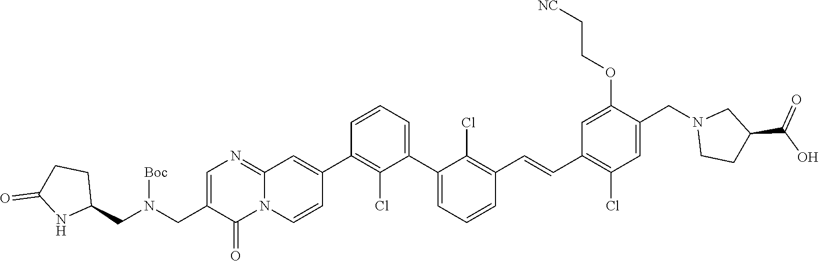 [SMILES: CC(C)(C)OC(=O)N(Cc1cnc2cc(-c3cccc(-c4cccc(/C=C/c5cc(OCCC#N)c(CN6CC[C@H](C(=O)O)C6)cc5Cl)c4Cl)c3Cl)ccn2c1=O)C[C@@H]1CCC(=O)N1]

To a mixture of (S,E)-tert-butyl ((8-(2,2′-dichloro-3′-(2-chloro-5-(2-cyanoethoxy)-4-formylstyryl)-[1,1′-biphenyl]-3-yl)-4-oxo-4H-pyrido[1,2-a]pyrimidin-3-yl)methyl)((5-oxopyrrolidin-2-yl)methyl)carbamate (0.19 g, 0.22 mmol, 45% purity) and (3S)-pyrrolidine-3-carboxylic acid (0.05 g, 0.45 mmol) in MeOH/CH₂Cl₂ mixture (3:1, 4 mL) was added sodium acetate (0.06 g, 0.67 mmol) and sodium triacetoxyborohydride (0.14 g, 0.67 mmol) in one portion. The mixture was stirred at rt for 16 h under N₂ atmosphere. The mixture was concentrated to afford (S)-1-(4-((E)-2-(3′-(3-(((tert-butoxycarbonyl)(((S)-5-oxopyrrolidin-2-yl)methyl)amino)methyl)-4-oxo-4H-pyrido[1,2-a]pyrimidin-8-yl)-2,2′-dichloro-[1,1′-biphenyl]-3-yl)vinyl)-5-chloro-2-(2-cyanoethoxy)benzyl)pyrrolidine-3-carboxylic acid as a yellow solid (0.35 g, crude, m/z: 925 [M+H]⁺ observed).

### (S)-1-(5-Chloro-2-(2-cyanoethoxy)-4-((E)-2-(2,2′-dichloro-3′-(4-oxo-3-(((((S)-5-oxopyrrolidin-2-yl)methyl)amino)methyl)-4H-pyrido[1,2-a]pyrimidin-8-yl)-[1,1′-biphenyl]-3-yl)vinyl)benzyl)pyrrolidine-3-carboxylic acid

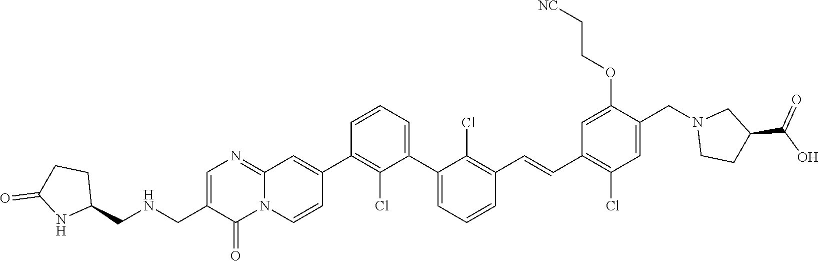 [SMILES: N#CCCOc1cc(/C=C/c2cccc(-c3cccc(-c4ccn5c(=O)c(CNC[C@@H]6CCC(=O)N6)cnc5c4)c3Cl)c2Cl)c(Cl)cc1CN1CC[C@H](C(=O)O)C1]

A mixture of(S)-1-(4-((E)-2-(3′-(3-(((tert-butoxycarbonyl)(((S)-5-oxopyrrolidin-2-yl)methyl)amino)methyl)-4-oxo-4H-pyrido[1,2-a]pyrimidin-8-yl)-2,2′-dichloro-[1,1′-biphenyl]-3-yl)vinyl)-5-chloro-2-(2-cyanoethoxy)benzyl)pyrrolidine-3-carboxylic acid (0.35 g, 0.38 mmol) in trifluoroacetic acid (5 mL, 67.5 mmol) was stirred at rt for 1 h and then at 40° C. for 1 h. The mixture was concentrated and the residue was purified by reverse phase HPLC to afford (S)-1-(5-chloro-2-(2-cyanoethoxy)-4-((E)-2-(2,2′-dichloro-3′-(4-oxo-3-(((((S)-5-oxopyrrolidin-2-yl)methyl)amino)methyl)-4H-pyrido[1,2-a]pyrimidin-8-yl)-[1,1′-biphenyl]-3-yl)vinyl)benzyl)pyrrolidine-3-carboxylic acid as a white solid (15 mg, 4% yield, m/z: 825 [M+H]⁺ observed). ¹H NMR (400 MHz, DMSO-d₆): δ 9.13 (d, J=7.6 Hz, 1H), 8.57 (s, 1H), 7.96-7.92 (m, 2H), 7.73-7.55 (m, 8H), 7.48-7.42 (m, 2H), 4.43 (t, J=6 Hz, 2H), 4.28-4.20 (m, 3H), 3.88-3.86 (m, 1H), 3.25 (m, 4H), 3.12-3.01 (m, 6H), 2.23-2.09 (m, 5H), 1.81-1.46 (m, 1H).

### Example 258: (S)-5-((((6-(2,2′-Dichloro-3′-(3-(((((S)-5-oxopyrrolidin-2-yl)methyl)amino)methyl)-1,6-naphthyridin-7-yl)-[1,1′-biphenyl]-3-yl)-2-methoxypyridin-3-yl)methyl)amino)methyl) pyrrolidin-2-one

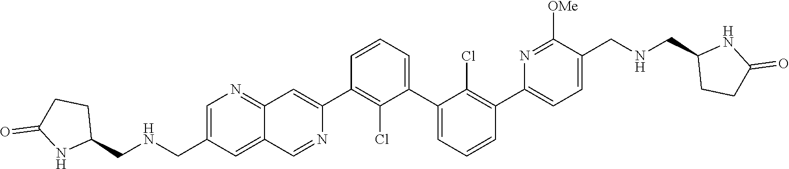 [SMILES: COc1nc(-c2cccc(-c3cccc(-c4cc5ncc(CNC[C@@H]6CCC(=O)N6)cc5cn4)c3Cl)c2Cl)ccc1CNC[C@@H]1CCC(=O)N1]

### 3-Bromo-7-chloro-1,6-naphthyridine

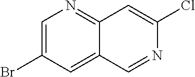 [SMILES: Clc1cc2ncc(Br)cc2cn1]

A mixture of 4-amino-6-chloro-pyridine-3-carbaldehyde (5 g, 31.93 mmol), 2-bromo-1, 1-dimethoxy-ethane (16.19 g, 95.80 mmol, 11.24 mL) and tris(trifluoromethylsulfonly oxy)ytterbium (4.95 g, 7.98 mmol) in acetonitrile (50 mL) was degassed and purged with N₂ and stirred at 80° C. for 24 h under N₂ atmosphere. The mixture was cooled to rt and filtered. The filter cake was rinsed with ethyl acetate (15 mL×3) and dried in vacuo to give desired product (3 g). To the above filtrate was added water (50 mL) and extracted with EtOAc (3×). The combined organic phase was washed with brine (15 mL×2), dried with anhydrous sodium sulfate, filtered and concentrated in vacuo. The residue was purified by normal phase SiO₂ chromatography (0-100% EtOAc/petroleum ether) to afford 3-bromo-7-chloro-1,6-naphthyridine (7 g, 90% yield) as a white solid. ¹H NMR (400 MHz, DMSO-D₆): δ9.27 (s, 1H), 9.22 (s, 1H), 8.98 (s, 1H), 8.11 (s, 1H). 7-Chloro-3-vinyl-1,6-naphthyridine

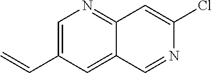

To a mixture of 3-bromo-7-chloro-1,6-naphthyridine (1.53 g, 6.28 mmol) in dioxane/water (10/1, 44 mL) was added 4,4,5,5-tetramethyl-2-vinyl-1,3,2-dioxaborolane (1.17 mL, 6.91 mmol), potassium phosphate (4.00 g, 18.8 mmol) and 1,1′-bis(diphenylphosphino)ferrocene-palladium(ii) dichloride dichloromethane complex (513 mg, 0.63 mmol) under N₂. The reaction was stirred for 2 h at 100° C. After cooling, the reaction was diluted with water (80 mL) and extracted with EtOAc (60 mL×3). The organic layer was dried over sodium sulfate, filtered and concentrated. The residue was purified by normal phase SiO₂ chromatography (0-30% EtOAc/petroleum ether) to afford 7-chloro-3-vinyl-1,6-naphthyridine (0.6 g, 50% yield) as a white solid. ¹H NMR (400 MHz, CDCl₃): δ 9.12 (s, 1H), 9.01 (s, 1H), 8.11 (s, 1H), 7.90 (s, 1H), 6.85-6.78 (dd, J=11.2 Hz, J=17.6 Hz, 1H), 6.00 (d, J=17.6 Hz, 1H), 5.51 (d, J=11.2 Hz, 1H).

### 7-Chloro-1,6-naphthyridine-3-carbaldehyde

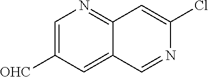 [SMILES: O=Cc1cnc2cc(Cl)ncc2c1]

To a solution of 7-chloro-3-vinyl-1,6-naphthyridine (750 mg, 3.93 mmol) in THF/water (4/1, 75 mL) at 0° C. was added dipotassium;dioxido(dioxo)osmium;dihydrate (29.0 mg, 0.079 mmol) and sodium periodate (3.37 g, 15.7 mmol). The reaction was stirred for 12 h at 20° C. The reaction was diluted with water (20 mL) and extracted with EtOAc (3×). The organic layer was dried over Na₂SO₄, filtered and concentrated in vacuo. The residue was purified by normal phase SiO₂ chromatography (0-30% EtOAc/petroleum ether) to afford 7-chloro-1,6-naphthyridine-3-carbaldehyde (670 mg, 88% yield) as a white solid. ¹H NMR (400 MHz, CDCl₃): δ 10.28 (s, 1H), 9.54 (s, 1H), 9.28 (s, 1H), 8.78 (s, 1H), 8.10 (s, 1H).

### 7-(2,2′-Dichloro-3′-(5-formyl-6-methoxypyridin-2-yl)-[1,1′-biphenyl]-3-yl)-1,6-naphthyridine-3-carbaldehyde

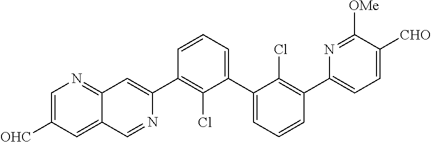 [SMILES: COc1nc(-c2cccc(-c3cccc(-c4cc5ncc(C=O)cc5cn4)c3Cl)c2Cl)ccc1C=O]

To a mixture of 7-chloro-1,6-naphthyridine-3-carbaldehyde (150 mg, 0.78 mmol) and 6-(2,2′-dichloro-3′-(4,4,5,5-tetramethyl-1,3,2-dioxaborolan-2-yl)-[1,1′-biphenyl]-3-yl)-2-methoxynicotinaldehyde (453 mg, 0.93 mmol) in DME/water (5/1, 24 mL) was added tetrakis(triphenylphosphine)palladium(0) (45 mg, 38.9 umol) and sodium carbonate (248 mg, 2.34 mmol). The reaction was stirred for 4 h at 80° C. After cooling, water (50 mL) was added to the reaction mixture and extracted with EtOAc (200 mL). The organic layer was dried over sodium sulfate, filtered and concentrated in vacuo. The residue was purified by normal phase SiO₂ chromatography (0-50% EtOAc/petroleum ether) to afford 7-(2,2′-dichloro-3′-(5-formyl-6-methoxypyridin-2-yl)-[1,1′-biphenyl]-3-yl)-1,6-naphthyridine-3-carbaldehyde (297 mg, 56% yield) as a light-yellow solid. ¹H NMR (400 MHz, CDCl₃): δ 10.43 (s, 1H), 10.31 (s, 1H), 9.57 (d, J=9.2 Hz, 2H), 8.83 (d, J=2.0 Hz, 1H), 8.42 (s, 1H), 8.19 (d, J=4.0 Hz, 1H), 7.68 (d, J=7.6 Hz, 1H), 7.52 (d, J=7.6 Hz, 1H), 7.48-7.26 (m, 5H), 4.14 (s, 3H).

### (S)-5-((((6-(2,2′-Dichloro-3′-(3-(((((S)-5-oxopyrrolidin-2-yl)methyl)amino)methyl)-1,6-naphthyridin-7-yl)-[1,1′-biphenyl]-3-yl)-2-methoxypyridin-3-yl)methyl)amino)methyl)pyrrolidin-2-one

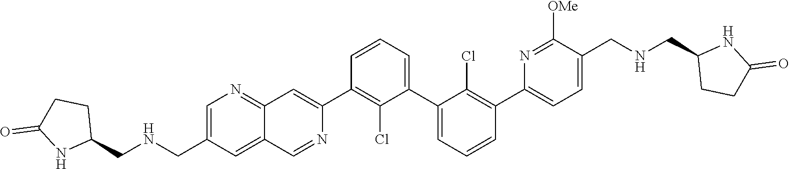 [SMILES: COc1nc(-c2cccc(-c3cccc(-c4cc5ncc(CNC[C@@H]6CCC(=O)N6)cc5cn4)c3Cl)c2Cl)ccc1CNC[C@@H]1CCC(=O)N1]

To a mixture of 7-(2,2′-dichloro-3′-(5-formyl-6-methoxypyridin-2-yl)-[1,1′-biphenyl]-3-yl)-1,6-naphthyridine-3-carbaldehyde (150 mg, 0.29 mmol) in DCM/MeOH (1/1, 20 mL) was added (5S)-5-(aminomethyl)pyrrolidin-2-one, HCl (176 mg, 1.17 mmol), sodium acetate (144 mg, 1.75 mmol) and stirred for 2 h at 30° C. Sodium cyanoborohydride (73 mg, 1.17 mmol) was added and the reaction mixture was stirred for 1 h at 30° C. The reaction mixture was concentrated in vacuo and the residue was purified by reverse phase HPLC to afford (5S)-5-[[[6-[2-chloro-3-[2-chloro-3-[3-[[[(2S)-5-oxopyrrolid in-2-yl]methylamino]methyl]-1,6-naphthyridin-7-yl]phenyl]phenyl]-2-methoxy-3-pyridyl]methylamino]methyl]pyrrolidin-2-one as a white solid (36 mg, 16% yield). m/z: 710 [M]⁺, 712 [M+2]⁺ observed. ¹H NMR (400 MHz, DMSO-D₆): δ9.50 (s, 1H), 9.17 (d, J=2.0 Hz, 1H), 8.53 (s, 1H), 8.16 (d, J=7.2 Hz, 2H), 7.84 (d, J=7.6 Hz, 1H), 7.76-7.69 (d, J=1.6 Hz, J=25.6 Hz, 1H), 7.68-7.67 (m, 3 H), 7.61-7.47 (m, 4H), 7.29 (d, J=7.2 Hz, 1H), 3.99 (d, J=3.2 Hz, 2H), 3.93 (s, 3H), 3.74 (d, J=1.6 Hz, 2H), 3.67-3.64 (m, 2H), 2.58-2.56 (m, 4H), 2.15-2.08 (m, 6H), 1.71-1.70 (m, 2H).

### Example 398: (S)-5-((((8-Chloro-2-(2,2′-dichloro-3′-(4-fluoro-6-methoxy-5-(((((S)-5-oxopyrrolidin-2-yl)methyl)amino)methyl)pyridin-2-yl)-[1,1′-biphenyl]-3-yl)-5-methoxyquinolin-6-yl)methyl)amino)methyl)pyrrolidin-2-one

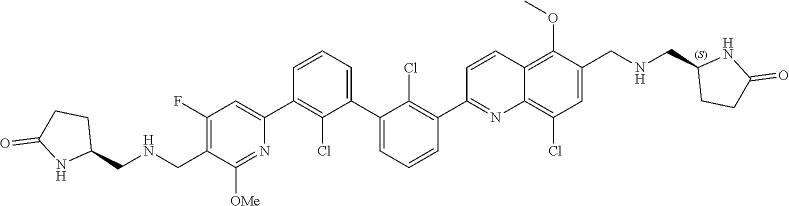

### 6-(3-Bromo-2-chlorophenyl)-4-fluoro-2-methoxynicotinaldehyde

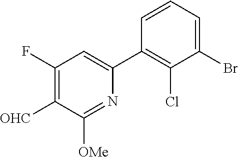 [SMILES: COc1nc(-c2cccc(Br)c2Cl)cc(F)c1C=O]

To a mixture of 6-chloro-4-fluoro-2-methoxynicotinaldehyde (4 g, 21 mmol) and 2-(3-bromo-2-chlorophenyl)-4,4,5,5-tetramethyl-1,3,2-dioxaborolane (6.4 g, 20 mmol) in dioxane/water mixture (5:1, 96 mL) was added potassium carbonate (8.75 g, 63 mmol) and tetrakis(triphenylphosphine)palladium (2.4 g, 2 mmol). The mixture was stirred at 100° C. for 2 h under N₂. The mixture was concentrated, and the residue was suspended in water (15 mL). The mixture was extracted with EtOAc (2×50 mL). The combined organic phase was dried with anhydrous sodium sulfate, filtered and concentrated. The residue was purified by normal phase SiO₂ chromatography (0-10% EtOAc/petroleum ether) to afford 6-(3-bromo-2-chlorophenyl)-4-fluoro-2-methoxynicotinaldehyde as a white solid (3 g, 62%).

### 6-(2-Chloro-3-(4,4,5,5-tetramethyl-1,3,2-dioxaborolan-2-yl)phenyl)-4-fluoro-2-methoxynicotinaldehyde

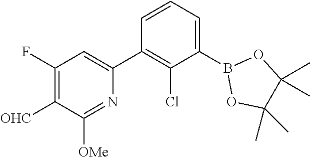 [SMILES: COc1nc(-c2cccc(B3OC(C)(C)C(C)(C)O3)c2Cl)cc(F)c1C=O]

To a mixture of 6-(3-bromo-2-chlorophenyl)-4-fluoro-2-methoxynicotinaldehyde (0.5 g, 1.45 mmol) and bis(pinacolato)diboron (0.66 g, 2.6 mmol) in dioxane (1 mL) was added potassium acetate (0.28 mg, 2.9 mmol) and 1,1′-bis(diphenylphosphino)ferrocene-palladium(ii) dichloride dichloromethane complex (59 mg, 72 umol). The mixture was stirred at 95° C. for 2 h under N₂. The mixture was filtered, and the filtrate was concentrated to give the residue. The residue was purified by normal phase SiO₂ chromatography (0-10% EtOAc/petroleum ether) to afford 6-(2-chloro-3-(4,4,5,5-tetramethyl-1,3,2-dioxaborolan-2-yl)phenyl)-4-fluoro-2-methoxynicotinaldehyde as a white solid (0.5 g, 66%). ¹H NMR (400 MHz, CDCl₃): δ10.32 (s, 1H), 7.66 (d, J=7.2 Hz, 1H), 7.54 (d, J=9.2 Hz, 1H), 7.29 (t, J=7.6 Hz, 1H), 7.02 (d, J=10.4 Hz, 1H), 4.03 (s, 3H), 1.32 (s, 12H). 6-(3′-Bromo-2,2′-dichloro-[1,1′-biphenyl]-3-yl)-4-fluoro-2-methoxynicotinaldehyde

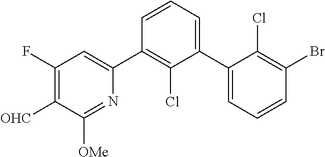 [SMILES: COc1nc(-c2cccc(-c3cccc(Br)c3Cl)c2Cl)cc(F)c1C=O]

To a mixture of 6-(2-chloro-3-(4,4,5,5-tetramethyl-1,3,2-dioxaborolan-2-yl)phenyl)-4-fluoro-2-methoxynicotinaldehyde (0.4 g, 1 mmol) and 1,3-dibromo-2-chlorobenzene (0.83 g, 3 mmol) in dioxane/water mixture (6:1, 1.4 mL) was added cesium carbonate (1 g, 3 mmol) and tetrakis(triphenylphosphine)palladium (59 mg, 51 umol). The mixture was stirred at 80° C. for 4 h under N₂. The mixture was concentrated to give the residue. The residue was purified by normal phase SiO₂ chromatography (0-5% EtOAc/petroleum ether) to afford 6-(3′-bromo-2,2′-dichloro-[1,1′-biphenyl]-3-yl)-4-fluoro-2-methoxynicotinaldehyde s a white solid (0.3 g, 64% yield)¹H NMR (400 MHz, CDCl₃): δ10.33 (s, 1H), 7.65-7.61 (m, 2H), 7.40 (t, J=7.2 Hz, 1H), 7.21 (d, J=7.2 Hz, 1H), 7.19-7.09 (m, 3H), 4.07 (s, 3H).

### 6-(2,2′-Dichloro-3′-(4,4,5,5-tetramethyl-1,3,2-dioxaborolan-2-yl)-[1,1′-biphenyl]-3-yl)-4-fluoro-2-methoxynicotinaldehyde

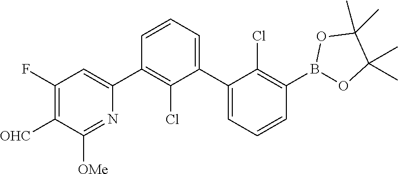 [SMILES: COc1nc(-c2cccc(-c3cccc(B4OC(C)(C)C(C)(C)O4)c3Cl)c2Cl)cc(F)c1C=O]

A mixture of 6-(3′-bromo-2,2′-dichloro-[1,1′-biphenyl]-3-yl)-4-fluoro-2-methoxynicotinaldehyde (0.3 g, 0.66 umol), bis(pinacolato)diboron (0.3 g, 1.2 mmol), potassium acetate (0.2 g, 2 mmol) and 1,1′-bis(diphenylphosphino)ferrocene-palladium(ii) dichloride dichloromethane complex (27 mg, 33 umol) in dioxane (3 mL) was stirred at 95° C. for 2 h under N₂. The mixture was filtered, and the filtrate was concentrated. The residue was purified by normal phase SiO₂ chromatography (0-10% EtOAc/petroleum ether) to afford 6-(2,2′-dichloro-3′-(4,4,5,5-tetramethyl-1,3,2-dioxaborolan-2-yl)-[1,1′-biphenyl]-3-yl)-4-fluoro-2-methoxynicotinaldehyde as a white solid (0.15 g, 45% yield)¹H NMR (400 MHz, CDCl₃): δ10.32 (s, 1H), 7.65 (t, J=5.2 Hz, 1H), 7.58 (d, J=6 Hz, 1H), 7.36 (d, J=7.2 Hz, 1H), 7.27-7.19 (m, 3H), 7.12 (d, J=10.4 Hz, 1H), 4.07 (s, 3H), 1.32 (s, 12H).

### 8-Chloro-2-(2,2′-dichloro-3′-(4-fluoro-5-formyl-6-methoxypyridin-2-yl)-[1,1′-biphenyl]-3-yl)-5-methoxyquinoline-6-carbaldehyde

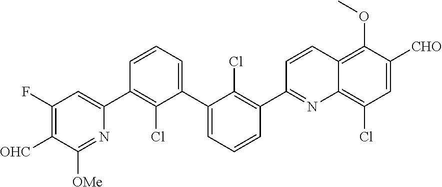 [SMILES: COc1nc(-c2cccc(-c3cccc(-c4ccc5c(OC)c(C=O)cc(Cl)c5n4)c3Cl)c2Cl)cc(F)c1C=O]

A mixture of 6-(2,2′-dichloro-3′-(4,4,5,5-tetramethyl-1,3,2-dioxaborolan-2-yl)-[1,1′-biphenyl]-3-yl)-4-fluoro-2-methoxynicotinaldehyde (0.15 g, 0.3 mmol), 2,8-dichloro-5-methoxyquinoline-6-carbaldehyde (76 mg, 0.3 mmol), 2,8-dichloro-5-methoxyquinoline-6-carbaldehyde (10 mg, 15 umol) and potassium phosphate (0.16 g, 0.75 mmol) in dioxane/water mixture (10:1, 2.2 mL) was stirred at 110° C. for 0.5 h under N₂. The mixture was concentrated, and the residue was purified by prep-TLC (SiO₂, petroleum ether:EtOAc=1:1) to afford 8-chloro-2-(2,2′-dichloro-3′-(4-fluoro-5-formyl-6-methoxypyridin-2-yl)-[1,1′-biphenyl]-3-yl)-5-methoxyquinoline-6-carbaldehyde as a yellow solid (0.12 g, 53% yield, m/z: 597 [M+H]⁺).

### (S)-5-((((8-Chloro-2-(2,2′-dichloro-3′-(4-fluoro-6-methoxy-5-(((((S)-5-oxopyrrolidin-2-yl)methyl)amino)methyl)pyridin-2-yl)-[1,1′-biphenyl]-3-yl)-5-methoxyquinolin-6-yl)methyl)amino)methyl)pyrrolidin-2-one

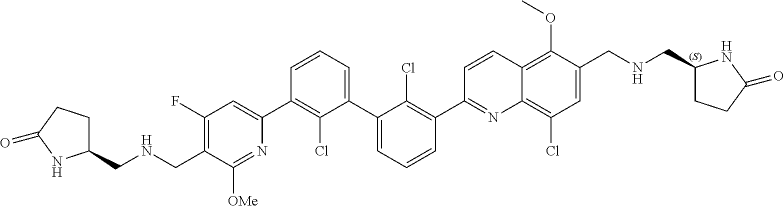

To a mixture of 8-chloro-2-(2,2′-dichloro-3′-(4-fluoro-5-formyl-6-methoxypyridin-2-yl)-[1,1′-biphenyl]-3-yl)-5-methoxyquinoline-6-carbaldehyde (0.12 g, 0.2 mmol) and (S)-5-(aminomethyl)pyrrolidin-2-one, HCl (0.1 g, 0.7 mmol) in DCM (2 mL) was added sodium acetate (83 mg, 1 mmol). The mixture was stirred at rt for 3 h under N₂. Sodium triacetyloxyboranuide (0.17 g, 0.81 mmol) was added and the mixture was stirred at rt for 2 h under N₂. The mixture was filtered and the filtrate was concentrated to give the residue. The residue was purified by reverse phase HPLC to afford (S)-5-((((8-chloro-2-(2,2′-dichloro-3′-(4-fluoro-6-methoxy-5-(((((S)-5-oxopyrrolidin-2-yl)methyl)amino)methyl)pyridin-2-yl)-[1,1′-biphenyl]-3-yl)-5-methoxyquinolin-6-yl)methyl)amino)methyl)pyrrolidin-2-one as a white solid (43.5 mg, 25% yield, m/z: 791 [M+H]⁺ observed). ¹H NMR (400 MHz, Methanol-d₄): δ. 8.65 (d, J=8.8 Hz, 1H), 8.06 (s, 1H), 7.96 (d, J=8.4 Hz, 1H), 7.77 (d, J=7.2 Hz, 1H), 7.69 (d, J=7.4 Hz, 1H), 7.62-7.47 (m, 4H), 7.18 (d, J=9.2 Hz, 1H), 4.15-3.95 (m, 11H), 3.90-3.89 (m, 1H), 2.88-2.80 (m, 4H), 2.41-2.27 (m, 6H), 1.87-1.82 (m, 2H). 1H NMR (400 MHz, DMSO-D₆): δ8.67 (d, J=8.4 Hz, 1H), 8.23-8.19 (m, 2H), 8.00-7.98 (d, J=8.8 Hz, 1H), 7.82-7.59 (m, 8H), 7.31 (d, J=9.2 Hz, 1H), 4.02-4.00 (m, 8H), 3.84-3.65 (m, 4H), 2.62-2.60 (m, 4H), 2.20-2.12 (m, 6H), 1.76-1.72 (m, 2H).

### Example 183: 8-(2,2′-Dichloro-3′-(6-methoxy-5-(((((S)-5-oxopyrrolidin-2-yl)methyl)amino)methyl)pyridin-2-yl)-[1,1′-biphenyl]-3-yl)-6-methyl-3-(((((S)-5-oxopyrrolidin-2-yl)methyl)amino)methyl)-4H-pyrido[1,2-a]pyrimidin-4-one

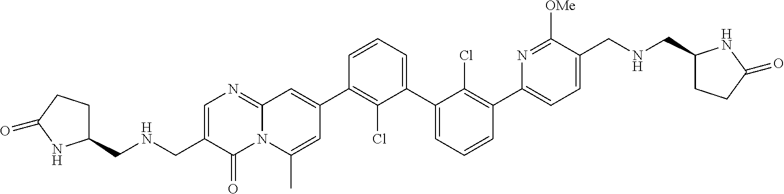 [SMILES: COc1nc(-c2cccc(-c3cccc(-c4cc(C)n5c(=O)c(CNC[C@@H]6CCC(=O)N6)cnc5c4)c3Cl)c2Cl)ccc1CNC[C@@H]1CCC(=O)N1]

### Diethyl 2-(((4-bromo-6-methylpyridin-2-yl)amino)methylene)malonate

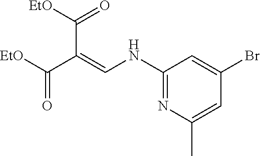 [SMILES: CCOC(=O)C(=CNc1cc(Br)cc(C)n1)C(=O)OCC]

A mixture of diethyl 2-(ethoxymethylene)malonate (11 mL, 55.9 mmol) and 4-bromo-6-methylpyridin-2-amine (9.5 g, 50.8 mmol) was stirred at 120° C. for 15 h. The mixture was cooled to 80° C. and poured into petroleum ether (100 mL) slowly at rt with stirring. The resulting yellow solid was collected by filtration. The solid was washed with petroleum ether (3×50 mL) to afford diethyl 2-(((4-bromo-6-methylpyridin-2-yl)amino)methylene)malonate (16.5 g, 90% yield). ¹H NMR (400 MHz, CDCl₃): δ 11.03 (d, J=12 Hz, 1H), 9.11 (d, J=12.8 Hz, 1H), 7.05 (s, 1H), 6.86 (s, 1H), 4.35-4.25 (m, 4H), 2.48 (s, 3H), 1.40-1.31 (m, 6H).

### Ethyl 8-bromo-6-methyl-4-oxo-4H-pyrido[1,2-a]pyrimidine-3-carboxylate

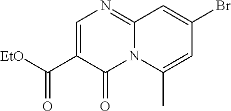 [SMILES: CCOC(=O)c1cnc2cc(Br)cc(C)n2c1=O]

A mixture of diethyl 2-(((4-bromo-6-methylpyridin-2-yl)amino)methylene)malonate (5 g, 14 mmol), polyphosphoric acid (0.9 g) and phosphorus oxybromide (7.7 mL, 75.6 mmol) was stirred at 130° C. for 1 h. The mixture was cooled to 80° C. followed by dropwise addition of EtOH (25 mL). The mixture was stirred at 80° C. for 1 h. To the mixture was added saturated aqueous sodium bicarbonate solution carefully until no gas comes out (pH 8). The resulting yellow solid was collected by filtration. The solid was washed with water (3×100 mL) and petroleum ether/ethyl acetate mixture (2:1, 2×50 mL), and dried in vacuo to afford ethyl 8-bromo-6-methyl-4-oxo-4H-pyrido[1,2-a]pyrimidine-3-carboxylate as a yellow solid (4.6 g, crude). ¹H NMR (400 MHz, CDCl₃): δ 8.64 (s, 1H), 7.85 (s, 1H), 7.43 (s, 1H), 4.23 (q, J=7.2 Hz, 2H), 2.91 (s, 3H), 1.27 (t, J=7.2 Hz, 3H).

### 8-Bromo-6-methyl-4-oxo-4H-pyrido[1,2-a]pyrimidine-3-carboxylic acid

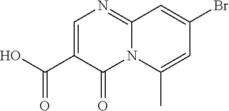 [SMILES: Cc1cc(Br)cc2ncc(C(=O)O)c(=O)n12]

A mixture of ethyl 8-bromo-6-methyl-4-oxo-4H-pyrido[1,2-a]pyrimidine-3-carboxylate (2.2 g, 7.07 mmol) in concentrated HCl (10 mL) was stirred at 50° C. for 16 h. The reaction was diluted with water (20 mL) and cooled to 5° C. The resulting yellow solid was collected by filtration to afford 8-bromo-6-methyl-4-oxo-4H-pyrido[1,2-a]pyrimidine-3-carboxylic acid (1.3 g, 64% yield, m/z: 283 [M]⁺, 285 [M+2]⁺ observed).

### 8-Bromo-3-(hydroxymethyl)-6-methyl-4H-pyrido[1,2-a]pyrimidin-4-one

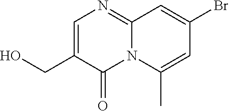

A mixture of 8-bromo-6-methyl-4-oxo-4H-pyrido[1,2-a]pyrimidine-3-carboxylic acid (1.1 g, 3.89 mmol) and N,N-carbonyldiimidazole (0.69 g, 4.27 mmol) in DMF (20 mL) was stirred at 45° C. for 1 h. The mixture was cooled to −10° C. followed by dropwise addition of sodium borohydride (0.18 g, 4.65 mmol) in water (3 mL). The mixture was stirred at −10° C. for 1 h. 1N aqueous HCl solution (2 mL) was added to the mixture at −10° C. The mixture was diluted with water (50 mL). The resulting yellow solid was collected by filtration to afford 8-bromo-3-(hydroxymethyl)-6-methyl-4H-pyrido[1,2-a]pyrimidin-4-one (0.48 g, 39% yield, m/z: 269 [M]⁺, 271 [M+2]⁺ observed). ¹H NMR (400 MHz, DMSO-d₆): δ 8.08 (s, 1H), 7.65 (d, J=2 Hz, 1H), 7.11 (d, J=1.2 Hz, 1H), 5.12 (t, J=1.2 Hz, 1H), 4.35 (d, J=4.8 Hz, 2H), 2.91 (s, 3H).

### 8-Bromo-6-methyl-4-oxo-4H-pyrido[1,2-a]pyrimidine-3-carbaldehyde

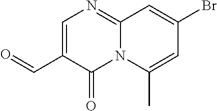

To a mixture of 8-bromo-3-(hydroxymethyl)-6-methyl-4H-pyrido[1,2-a]pyrimidin-4-one (0.48 g, 1.78 mmol) in CHCl₃ (30 mL) was added manganese(IV) oxide (0.93 g, 10.7 mmol) in one portion. The mixture was stirred at 50° C. for 12 h under N₂ atmosphere. The mixture was filtered. The filtrate was dried in vacuo to afford 8-bromo-6-methyl-4-oxo-4H-pyrido[1,2-a]pyrimidine-3-carbaldehyde as a yellow solid (0.33 g, 69% yield). ¹H NMR (400 MHz, DMSO-d₆): δ 10.03 (s, 1H), 8.55 (s, 1H), 7.95 (s, 1H), 7.55 (s, 1H), 3.01 (s, 3H).

### 8-(2,2′-Dichloro-3′-(5-formyl-6-methoxypyridin-2-yl)-[1,1′-biphenyl]-3-yl)-6-methyl-4-oxo-4H-pyrido[1,2-a]pyrimidine-3-carbaldehyde

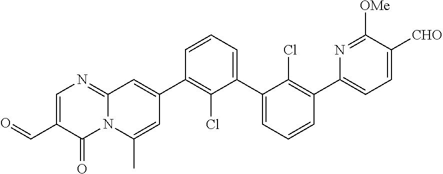 [SMILES: COc1nc(-c2cccc(-c3cccc(-c4cc(C)n5c(=O)c(C=O)cnc5c4)c3Cl)c2Cl)ccc1C=O]

To a mixture of 8-bromo-6-methyl-4-oxo-4H-pyrido[1,2-a]pyrimidine-3-carbaldehyde (0.3 g, 1.12 mmol) and 6-(2,2′-dichloro-3′-(4,4,5,5-tetramethyl-1,3,2-dioxaborolan-2-yl)-[1,1′-biphenyl]-3-yl)-2-methoxynicotinaldehyde (0.54 g, 1.12 mmol) in 1,4-dioxane/water mixture (5:1, 18 mL) was added 1,1′-bis(diphenylphosphino)ferrocene-palladium(ii) dichloride dichloromethane complex (0.05 g, 0.06 mmol) and potassium carbonate (0.47 g, 3.37 mmol) under N₂ atmosphere. The mixture was stirred at 110° C. for 0.5 h. The mixture was concentrated and the residue was suspended in water (50 mL) and brine (50 mL). The mixture was extracted with ethyl acetate/THF mixture (1:1, 100 mL). The organic phase was dried with anhydrous sodium sulfate, filtered and concentrated. The residue was purified by normal phase SiO₂ chromatography (0-50% 30% THF in ethyl acetate/petroleum ether) to afford 8-(2,2′-dichloro-3′-(5-formyl-6-methoxypyridin-2-yl)-[1,1′-biphenyl]-3-yl)-6-methyl-4-oxo-4H-pyrido[1,2-a]pyrimidine-3-carbaldehyde was obtained as a light-yellow solid (0.42 g, 62% yield).δ10.36 (s, 1H), 10.13 (s, 1H), 8.66 (s, 1H), 8.28 (d, J=7.6 Hz, 1H), 7.84-7.82 (m, 2H), 7.76-7.65 (m, 4H), 7.62-7.59 (m, 1H), 7.56-7.52 (m, 2H), 4.13 (s, 3H), 3.17 (s, 3H).

### 8-(2,2′-Dichloro-3′-(6-methoxy-5-(((((S)-5-oxopyrrolidin-2-yl)methyl)amino)methyl)pyridin-2-yl)-[1,1′-biphenyl]-3-yl)-6-methyl-3-(((((S)-5-oxopyrrolidin-2-yl)methyl)amino)methyl)-4H-pyrido[1,2-a]pyrimidin-4-one

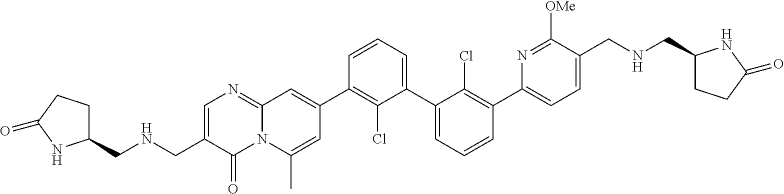 [SMILES: COc1nc(-c2cccc(-c3cccc(-c4cc(C)n5c(=O)c(CNC[C@@H]6CCC(=O)N6)cnc5c4)c3Cl)c2Cl)ccc1CNC[C@@H]1CCC(=O)N1]

To a mixture of 8-(2,2′-dichloro-3′-(5-formyl-6-methoxypyridin-2-yl)-[1,1′-biphenyl]-3-yl)-6-methyl-4-oxo-4H-pyrido[1,2-a]pyrimidine-3-carbaldehyde (0.25 g, 0.46 mmol) and (S)-5-(aminomethyl)-2-pyrrolidinone, HCl (0.24 g, 1.61 mmol) in MeOH/DCM (5:4, 9 mL) was added sodium acetate (0.19 g, 2.3 mmol) and sodium triacetoxyborohydride (0.58 g, 2.76 mmol) in one portion. The mixture was stirred at rt for 4 h under N₂ atmosphere. The mixture was concentrated. The residue was purified by reverse phase HPLC to afford 8-(2,2′-dichloro-3′-(6-methoxy-5-(((((S)-5-oxopyrrolidin-2-yl)methyl)amino)methyl)pyridin-2-yl)-[1,1′-biphenyl]-3-yl)-6-methyl-3-(((((S)-5-oxopyrrolidin-2-yl)methyl)amino)methyl)-4H-pyrido[1,2-a]pyrimidin-4-one as a white solid (122.7 mg, 34% yield, m/z: 740 [M+H]⁺ observed). ¹H NMR (400 MHz, DMSO-d₆): δ 8.20 (s, 1H), 7.83 (d, J=7.6 Hz, 1H), 7.72-7.61 (m, 7H), 7.48-7.45 (m, 2H), 7.28 (d, J=7.6 Hz, 1H), 7.07 (d, J=1.2 Hz, 1H), 3.92 (s, 3H), 3.76-3.58 (m, 6H), 3.01 (s, 3H), 2.55 (d, J=6 Hz, 2H), 2.19-2.03 (m, 8H), 1.75-1.65 (m, 2H).

### Example 228: (S)-5-((((6-(2,2′-Dichloro-3′-((6-methoxy-5-(((((S)-5-oxopyrrolidin-2-yl)methyl)amino) methyl)-2,3-dihydro-1H-inden-1-ylidene)methyl)-[1,1′-biphenyl]-3-yl)-2-methoxypyridin-3-yl)methyl)amino)methyl)pyrrolidin-2-one

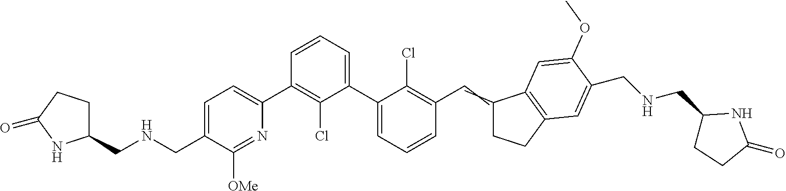 [SMILES: COc1cc2c(cc1CNC[C@@H]1CCC(=O)N1)CCC2=Cc1cccc(-c2cccc(-c3ccc(CNC[C@@H]4CCC(=O)N4)c(OC)n3)c2Cl)c1Cl]

### Methyl 6-methoxy-1-oxo-2,3-dihydro-1H-indene-5-carboxylate

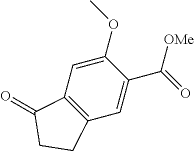 [SMILES: COC(=O)c1cc2c(cc1OC)C(=O)CC2]

To a mixture of 5-bromo-6-methoxy-2,3-dihydro-1H-inden-1-one (15 g, 62.2 mmol) in MeOH (150 mL) was added [1,1-Bis(diphenylphosphino)ferrocene]dichloropalladium(II) (4.55 g, 6.22 mmol) and triethylamine (26 mL, 187 mmol). The mixture was stirred at 80° C. for 24 h at 50 psi under CO. The mixture was filtered. The filtrate was concentrated and the residue was purified by normal phase SiO₂ chromatography (0-30% EtOAc/petroleum ether) to afford methyl 6-methoxy-1-oxo-indane-5-carboxylate (9 g, 65% yield) as a white solid. ¹H NMR (400 MHz, DMSO-d₆): δ 7.73 (s, 1H), 7.22 (s, 1H), 3.84 (s, 3H), 3.81 (s, 3H), 3.01 (d, J=4.8 Hz, 2H), 2.67 (t, J=4.8 Hz, 2H).

### (3-Bromo-2-chlorobenzyl)triphenylphosphonium bromide

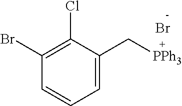

A mixture of 1-bromo-3-(bromomethyl)-2-chloro-benzene (7.5 g, 26.4 mmol) and triphenylphosphane (7.26 g, 27.7 mmol) in DCM (80 mL) was stirred at rt for 12 h under N₂. The mixture was concentrated, diluted with petroleum ether (30 mL) and the mixture was stirred at rt for 0.5 h. The suspension was filtered to afford (3-bromo-2-chlorobenzyl)triphenylphosphonium bromide (14.25 g, 98% yield) as a white solid. ¹H NMR (400 MHz, DMSO-d₆): δ 7.93-7.76 (m, 3H), 7.75-7.64 (m, 13H), 5.29 (d, J=15.2 Hz, 2H).

### Methyl1-(3-bromo-2-chlorobenzylidene)-6-methoxy-2,3-dihydro-1H-indene-5-carboxylate

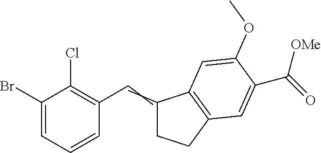 [SMILES: COC(=O)c1cc2c(cc1OC)C(=Cc1cccc(Br)c1Cl)CC2]

To a mixture of (3-bromo-2-chlorobenzyl)triphenylphosphonium bromide (2.98 g, 5.45 mmol) in THE (10 mL) was added n-butyllithium (2.18 mL, 2.5 M) at rt. After stirring the mixture for 2 h, 6-methoxy-1-oxo-indane-5-carboxylate (1 g, 4.54 mmol) in THE (10 mL) was added. The mixture was stirred at 80° C. for 24 h. The mixture was combined with another two batches at the same scale. The reaction was quenched with water (100 mL). The aqueous layer was extracted with ethyl acetate (2×300 mL). The combined organic phase was dried over anhydrous sodium sulfate, filtered and concentrated under reduced pressure. The residue was purified by normal phase SiO₂ chromatography (0-20% EtOAc/petroleum ether) to afford methyl 1-(3-bromo-2-chlorobenzylidene)-6-methoxy-2,3-dihydro-1H-indene-5-carboxylate (0.35 g, m/z: 407 [M]⁺, 409 [M+2]⁺, observed).

### Methyl-1-((2,2′-dichloro-3′-(5-formyl-6-methoxypyridin-2-yl)-[1,1′-biphenyl]-3-yl)methylene)-6-methoxy-2,3-dihydro-1H-indene-5-carboxylate

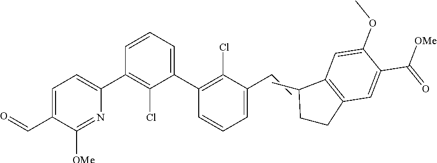

To a mixture of methyl 1-(3-bromo-2-chlorobenzylidene)-6-methoxy-2,3-dihydro-1H-indene-5-carboxylate (700 mg, 1.72 mmol), 6-(2-chloro-3-(4,4,5,5-tetramethyl-1,3,2-dioxaborolan-2-yl)phenyl)-2-methoxynicotinaldehyde (962 mg, 2.58 mmol) in dioxane/water mixture (5:1, 12 mL) was added potassium carbonate (712 mg, 5.15 mmol) and [1,1-bis(diphenylphosphino) ferrocene]palladium(II) chloride dichloromethane complex (140 mg, 0.17 mmol). The mixture was stirred at 80° C. for 1 h under N₂. The mixture was filtered, and the filtrate was concentrated. The residue obtained was purified by normal phase SiO₂ chromatography (0-30% EtOAc/petroleum ether) to afford methyl-1-((2,2′-dichloro-3′-(5-formyl-6-methoxypyridin-2-yl)-[1,1′-biphenyl]-3-yl)methylene)-6-methoxy-2,3-dihydro-1H-indene-5-carboxylate (800 mg, 81% yield) as a yellow solid. ¹H NMR (400 MHz, DMSO-d₆): δ 10.30 (m, 1H), 8.23-8.20 (m, 1H), 7.74-7.61 (m, 1H), 7.60-7.50 (m, 7H), 7.49-7.47 (m, 1H), 6.74-6.46 (m, 1H), 4.07-4.06 (m, 3H), 3.90 (s, 2H), 3.88 (s, 1H), 3.77 (s, 1H), 3.75 (s, 2H), 3.37 (s, 2H).

### (6-(2,2′-Dichloro-3′-((5-(hydroxymethyl)-6-methoxy-2,3-dihydro-1H-inden-1-ylidene) methyl)-[1,1′-biphenyl]-3-yl)-2-methoxypyridin-3-yl)methanol

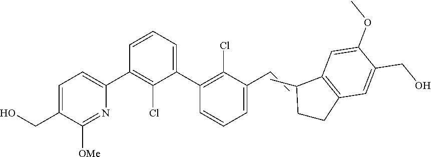

To a mixture of lithium aluminum hydride (0.22 mg, 5.92 mmol) in THE (10 mL), methyl-1-((2,2′-dichloro-3′-(5-formyl-6-methoxypyridin-2-yl)-[1,1′-biphenyl]-3-yl)methylene)-6-methoxy-2,3-dihydro-1H-indene-5-carboxylate (850 mg, 1.48 mmol) in THE (10 mL) was added at −30° C. The mixture was stirred at −30° C. for 2 h under N₂. The reaction was quenched with water (10 mL). The aqueous layer was extracted with EtOAc (2×20 mL). The organic layer was separated, dried over anhydrous sodium sulfate, filtered and concentrated to afford (6-(2,2′-dichloro-3′-((5-(hydroxymethyl)-6-methoxy-2,3-dihydro-1H-inden-1-ylidene)methyl)-[1,1′-biphenyl]-3-yl)-2-methoxypyridin-3-yl)methanol (600 mg, m/z: 548 [M+H]⁺ observed).

### 6-(2,2′-Dichloro-3′-((5-formyl-6-methoxy-2,3-dihydro-1H-inden-1-ylidene)methyl)-[1,1′-biphenyl]-3-yl)-2-methoxynicotinaldehyde

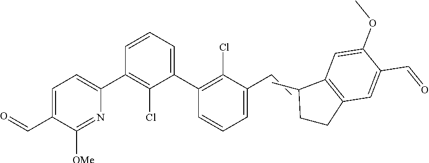

To a solution of (6-(2,2′-dichloro-3′-((5-(hydroxymethyl)-6-methoxy-2,3-dihydro-1H-inden-1-ylidene)methyl)-[1,1′-biphenyl]-3-yl)-2-methoxypyridin-3-yl)methanol (600 mg, 1.09 mmol) in CHCl₃ (20 mL) was added MnO₂ (1.90 g, 21.9 mmol). The mixture was stirred at 50° C. for 12 h.

The reaction was filtered and concentrated to afford 6-[2-chloro-3-[2-chloro-3-[(E)-(5-formyl-6-methoxy-indan-1-ylidene)methyl]phenyl]phenyl]-2-methoxy-pyridine-3-carbaldehyde (420 mg, crude) as a yellow solid. The crude product was used for the next step without purification. ¹H NMR (400 MHz, DMSO-d₆): δ 10.39-1.26 (m, 2H), 8.28-8.25 (m, 1H), 7.82-7.78 (m, 1H), 7.66-7.41 (m, 7H), 6.88 (s, 1H), 6.56 (s, 1H), 4.12-4.00 (m, 3H), 3.97-3.95 (m, 1H), 3.53 (s, 1H), 3.37-3.34 (m, 2H), 3.09-3.94 (m, 3H).

### (S)-5-((((6-(2,2′-Dichloro-3′-((6-methoxy-5-(((((S)-5-oxopyrrolidin-2-yl)methyl)amino) methyl)-2,3-dihydro-1H-inden-1-ylidene)methyl)-[1,1′-biphenyl]-3-yl)-2-methoxypyridin-3-yl)methyl)amino)methyl)pyrrolidin-2-one

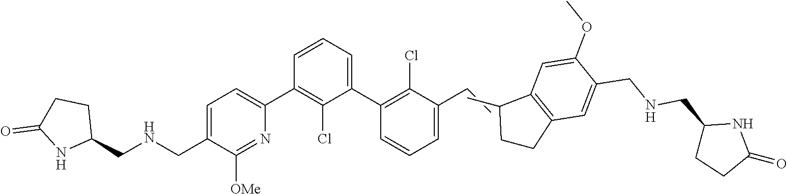

To a mixture of 6-[2-chloro-3-[2-chloro-3-[(E)-(5-formyl-6-methoxy-indan-1-ylidene)methyl]phenyl]phenyl]-2-methoxy-pyridine-3-carbaldehyde (200 mg, 0.37 mmol), (5S)-5-(aminomethyl)pyrrolidin-2-one, HCl (332 mg, 2.20 mmol) in DCM (10 mL) was added sodium acetate (181 mg, 2.20 mmol). After stirring at rt for 2 h, sodium triacetoxyborohydride (467 mg, 2.20 mmol) was added to the mixture and continued to stir at rt for 0.5 h. The mixture was concentrated and the residue was purified by reverse phase HPLC to afford (S)-5-((((6-(2,2′-dichloro-3′-((6-methoxy-5-(((((S)-5-oxopyrrolidin-2-yl)methyl)amino)methyl)-2,3-dihydro-1H-inden-1-ylidene)methyl)-[1,1′-biphenyl]-3-yl)-2-methoxypyridin-3-yl)methyl)amino) methyl)pyrrolidin-2-one (79.2 mg, m/z: 740 [M+H]⁺ observed). ¹H NMR (400 MHz, DMSO-d₆): δ 8.22 (s, 1H), 7.83-7.81 (m, 1H), 7.70-7.65 (m, 3H), 7.54-7.47 (m, 3H), 7.41-7.38 (m, 2H), 7.28-7.20 (m, 2H), 6.56-6.36 (m, 1H), 3.93-3.92 (m, 3H), 3.86 (s, 1H), 3.77 (s, 3H), 3.73-3.65 (m, 4H), 2.97-2.90 (m, 4H), 2.58-2.53 (m, 3H), 2.15-2.05 (m, 6H), 1.72-1.66 (m, 2H).

### Example 201: 8-(2,2′-Dichloro-3′-(6-methoxy-5-(((((S)-5-oxopyrrolidin-2-yl)methyl)amino)methyl)pyridin-2-yl)-[1,1′-biphenyl]-3-yl)-2-methoxy-3-(((((S)-5-oxopyrrolidin-2-yl)methyl)amino)methyl)-4H-pyrido[1,2-a]pyrimidin-4-one

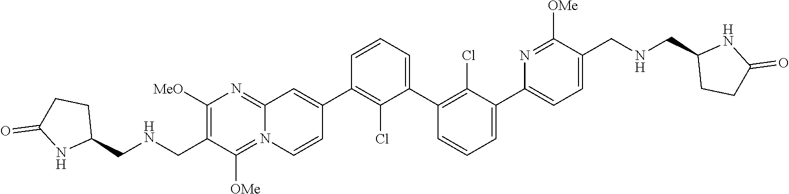

### ethyl 8-bromo-2-hydroxy-4-oxo-4H-pyrido[1,2-a]pyrimidine-3-carboxylate

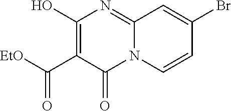 [SMILES: CCOC(=O)c1c(O)nc2cc(Br)ccn2c1=O]

A mixture of 4-bromopyridin-2-amine (30 g, 172 mmol) and triethyl methanetricarboxylate (54 mL, 258 mmol) was stirred at 120° C. for 12 h under N₂. Petroleum ether (150 mL) was added to the mixture and stirred at rt for 0.5 h. The mixture was filtered to give the filter cake. The solid was dried in vacuo to afford ethyl 8-bromo-2-hydroxy-4-oxo-4H-pyrido[1,2-a]pyrimidine-3-carboxylate as a red solid (25 g, 46% yield). ¹H NMR (400 MHz, DMSO-d₆): δ12.49 (br s, 1H), 8.76 (d, J=8 Hz, 1H), 7.56-7.53 (m, 2H), 4.15 (q, J=6.8 Hz, 2H), 1.21 (t, J=6.8 Hz, 3H).

### Ethyl 8-bromo-2-methoxy-4-oxo-4H-pyrido[1,2-a]pyrimidine-3-carboxylate

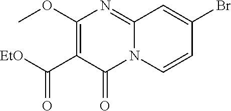 [SMILES: CCOC(=O)c1c(OC)nc2cc(Br)ccn2c1=O]

To a mixture of ethyl 8-bromo-2-hydroxy-4-oxo-4H-pyrido[1,2-a]pyrimidine-3-carboxylate (10 g, 32 mmol) and iodomethane (6 mL, 96 mmol) in DMF (80 mL) was added potassium carbonate (13 g, 96 mmol). The mixture was stirred at rt for 2.5 h under N₂. The mixture was diluted with saturated ammonium chloride solution (100 mL) and stirred at rt for 0.5 h. The mixture was extracted with ethyl acetate (150 mL×2). The combined organic phase was dried with anhydrous sodium sulfate, filtered and concentrated. The residue was purified by normal phase SiO₂ chromatography (0-35% ethyl acetate/petroleum ether) to afford ethyl 8-bromo-2-methoxy-4-oxo-4H-pyrido[1,2-a]pyrimidine-3-carboxylate as a yellow solid (1.8 g, 17% yield). ¹H NMR (400 MHz, DMSO-d₆): δ8.79 (d, J=7.6 Hz, 1H), 7.96 (d, J=1.6 Hz, 1H), 7.56 (dd, J=7.6 Hz, J=2 Hz, 1H), 4.21 (q, J=7.2 Hz, 2H), 3.95 (s, 3H), 1.24 (t, J=7.2 Hz, 3H).

### 8-Bromo-3-(hydroxymethyl)-2-methoxy-4H-pyrido[1,2-a]pyrimidin-4-one

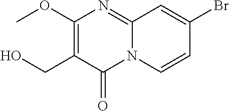 [SMILES: COc1nc2cc(Br)ccn2c(=O)c1CO]

To a mixture of ethyl 8-bromo-2-methoxy-4-oxo-4H-pyrido[1,2-a]pyrimidine-3-carboxylate (1.6 g, 5 mmol) in DCM (25 mL) at −10° C., bis(2-methylpropyl)alumanylium hydride (1 M in toluene, 12 mL, 12 mmol) was added. After stirring at rt. for 3 h, water (10 mL) was added to the mixture and stirring continued at 0° C. for 0.5 h and then at rt for 0.5 h. The mixture was filtered, and the filtrate was extracted with ethyl acetate (20 mL×2). The combined organic phase was dried with anhydrous sodium sulfate, filtered and concentrated. The residue was purified by normal phase SiO₂ chromatography (0-48% ethyl acetate/petroleum ether) to afford 8-bromo-3-(hydroxymethyl)-2-methoxy-4H-pyrido[1,2-a]pyrimidin-4-one as a yellow solid (0.5 g, 35% yield).

### 8-Bromo-2-methoxy-4-oxo-4H-pyrido[1,2-a]pyrimidine-3-carbaldehyde

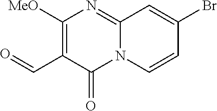 [SMILES: COc1nc2cc(Br)ccn2c(=O)c1C=O]

To a mixture of 8-bromo-3-(hydroxymethyl)-2-methoxy-4H-pyrido[1,2-a]pyrimidin-4-one (0.5 g, 1.75 mmol) in CHCl₃ (50 mL), manganese(IV) oxide (1.22 g, 14 mmol) was added. The mixture was stirred at 60° C. for 12 h under N₂. The mixture was filtered and the filtrate was concentrated to afford 8-bromo-2-methoxy-4-oxo-4H-pyrido[1,2-a]pyrimidine-3-carbaldehyde as a yellow solid (0.28 g, 56% yield). ¹H NMR (400 MHz, DMSO-d₆): δ10.14 (s, 1H), 8.88 (d, J=7.2 Hz, 1H), 7.99 (d, J=1.6 Hz, 1H), 7.64 (dd, J=4.4 Hz, J=1.6 Hz, 1H), 4.01 (s, 3H).

### 8-(2,2′-Dichloro-3′-(5-formyl-6-methoxypyridin-2-yl)-[1,1′-biphenyl]-3-yl)-2-methoxy-4-oxo-4H-pyrido[1,2-a]pyrimidine-3-carbaldehyde

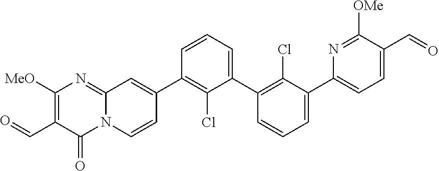 [SMILES: COc1nc(-c2cccc(-c3cccc(-c4ccn5c(=O)c(C=O)c(OC)nc5c4)c3Cl)c2Cl)ccc1C=O]

To a mixture of 8-bromo-2-methoxy-4-oxo-4H-pyrido[1,2-a]pyrimidine-3-carbaldehyde (0.1 g, 0.35 mmol) and 6-[2-chloro-3-[2-chloro-3-(4,4,5,5-tetramethyl-1,3,2-dioxaborolan-2-yl)phenyl]phenyl]-2-methoxy-pyridine-3-carbaldehyde (0.17 g, 0.35 mmol) in THF/water mixture (4:1, 10 mL), potassium carbonate (0.15 g, 1.06 mmol) and 1,1′-bis(diphenylphosphino)ferrocene-palladium(ii) dichloride dichloromethane complex (0.03 g, 0.04 mmol) was added. The mixture was stirred at 80° C. for 3 h. The mixture was diluted with water (10 mL). The mixture was extracted with ethyl acetate (25 mL×2). The combined organic phase was dried with anhydrous sodium sulfate, filtered and concentrated to afford 8-(2,2′-dichloro-3′-(5-formyl-6-methoxypyridin-2-yl)-[1,1′-biphenyl]-3-yl)-2-methoxy-4-oxo-4H-pyrido[1,2-a]pyrimidine-3-carbaldehyde as a brown oil (0.2 g, crude). ¹H NMR (400 MHz, CDCl₃): δ10.36 (s, 1H), 10.33 (s, 1H), 9.11 (d, J=7.2 Hz, 1H), 8.12 (d, J=7.6 Hz, 1H), 7.62-7.60 (m, 2H), 7.45-7.41 (m, 4H), 7.36-7.33 (m, 3H), 4.07 (s, 3H), 4.07 (s, 3H).

### 8-(2,2′-Dichloro-3′-(6-methoxy-5-(((((S)-5-oxopyrrolidin-2-yl)methyl)amino)methyl)pyridin-2-yl)-[1,1′-biphenyl]-3-yl)-2-methoxy-3-(((((S)-5-oxopyrrolidin-2-yl)methyl)amino)methyl)-4H-pyrido[1,2-a]pyrimidin-4-one

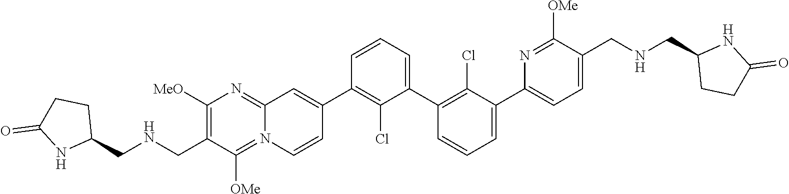

To a mixture of 8-(2,2′-dichloro-3′-(5-formyl-6-methoxypyridin-2-yl)-[1,1′-biphenyl]-3-yl)-2-methoxy-4-oxo-4H-pyrido[1,2-a]pyrimidine-3-carbaldehyde (0.2 g, 0.36 mmol) and (5S)-5-(aminomethyl)pyrrolidin-2-one, HCl (0.32 g, 2.14 mmol) in DCM (10 mL) was added sodium acetate (0.23 g, 2.86 mmol), sodium triacetyloxyboranuide (0.45 g, 2.14 mmol) and 4A molecular sieve (40 mg). The mixture was stirred at rt for 2 h. The mixture was filtered and the filtrate was concentrated. The residue was purified by reverse phase HPLC to afford 8-(2,2′-dichloro-3′-(6-methoxy-5-(((((S)-5-oxopyrrolidin-2-yl)methyl)amino)methyl)pyridin-2-yl)-[1,1′-biphenyl]-3-yl)-2-methoxy-3-(((((S)-5-oxopyrrolidin-2-yl)methyl)amino)methyl)-4H-pyrido[1,2-a]pyrimidin-4-one as a yellow solid (46.5 mg, 16% yield, m/z: 756 [M+H]⁺ observed). ¹H NMR (400 MHz, DMSO-d₆): δ9.00 (d, J=7.2 Hz, 1H), 7.81 (d, J=7.2 Hz, 1H), 7.67-7.53 (m, 8H), 7.48-7.45 (m, 2H), 7.26 (d, J=7.6 Hz, 1H), 3.99 (s, 3H), 3.91 (s, 3H), 3.64 (s, 4H), 3.63-3.55 (m, 2H), 2.54 (d, J=6.4 Hz, 2H), 2.46 (s, 2H) (overlapped with solvent peak), 2.13-2.02 (m, 6H), 1.69-1.64 (m, 2H).

### Example 262: 8-[2-Chloro-3-[2-chloro-3-[6-methoxy-5-[[[(2S)-5-oxopyrrolidin-2-yl]methylamino]methyl]-2-pyridyl]phphenlphenyl]-3-[[[(2S)-2-hydroxypropyl]amino]methyl]-2-methoxy-pyrido[1,2-a]pyrimidin-4-one

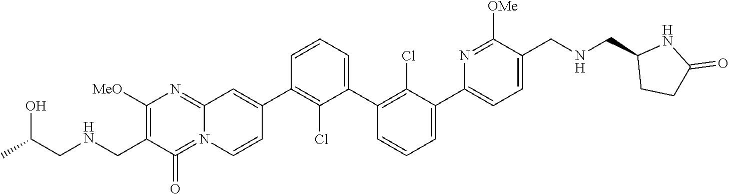

### (S)-tert-Butyl ((6-(2,2′-dichloro-3′-(3-formyl-2-methoxy-4-oxo-4H-pyrido[1,2-a]pyrimidin-8-yl)-[1,1′-biphenyl]-3-yl)-2-methoxypyridin-3-yl)methyl)((5-oxopyrrolidin-2-yl)methyl)carbamate

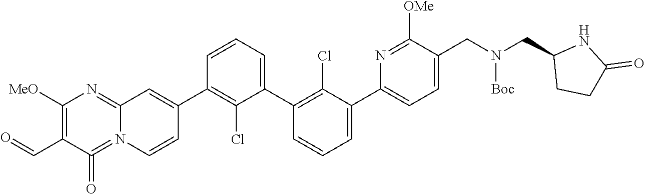

To a mixture of 8-(3-bromo-2-chloro-phenyl)-2-methoxy-4-oxo-pyrido[1,2-a]pyrimidine-3-carbaldehyde (0.65 g, 1.65 mmol) and (S)-tert-butyl ((6-(2,2′-dichloro-3′-(4,4,5,5-tetramethyl-1,3,2-dioxaborolan-2-yl)-[1,1′-biphenyl]-3-yl)-2-methoxypyridin-3-yl)methyl)((5-oxopyrrolidin-2-yl)methyl)carbamate (1.4 g, 2.5 mmol) in THF/water mixture (5.1, 24 mL) was added potassium carbonate (0.68 g, 5 mmol) and 1,1′-bis(diphenylphosphino)ferrocene-palladium(ii) dichloride dichloromethane complex (0.07 g, 0.08 mmol). The mixture was stirred at 80° C. for 3 h under N₂. The mixture was diluted with water (15 mL) and extracted with EtOAc (50 mL×2). The combined organic phase was dried with anhydrous sodium sulfate, filtered and concentrated. The residue was purified by normal phase SiO₂ chromatography (0-60% EtOAc/petroleum ether) to afford (S)-tert-butyl ((6-(2,2′-dichloro-3′-(3-formyl-2-methoxy-4-oxo-4H-pyrido[1,2-a]pyrimidin-8-yl)-[1,1′-biphenyl]-3-yl)-2-methoxypyridin-3-yl)methyl)((5-oxopyrrolidin-2-yl)methyl)carbamate (0.8 g, 51% yield) as a yellow solid. ¹H NMR (400 MHz, DMSO-d₆): δ10.21 (s, 1H), 9.12 (d, J=7.2 Hz, 1H), 7.78-7.76 (m, 2H), 7.76-7.60 (m, 5H), 7.58-7.50 (m, 2H), 7.31-7.30 (m, 1H), 4.43-4.35 (m, 2H), 4.07 (s, 3H), 3.95 (s, 3H), 3.79-3.77 (m, 2H), 2.18-2.06 (m, 3H), 1.74-1.73 (m, 1H), 1.44-1.31 (m, 9H).

### 8-(2,2′-Dichloro-3′-(6-methoxy-5-(((((S)-5-oxopyrrolidin-2-yl)methyl)amino)methyl)pyridin-2-yl)-[1,1′-biphenyl]-3-yl)-3-((((S)-2-hydroxypropyl)amino)methyl)-2-methoxy-4H-pyrido[1,2-a]pyrimidin-4-one

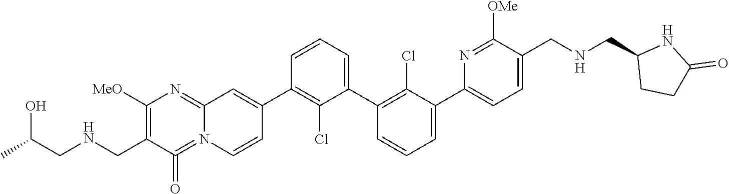

To a mixture of (S)-tert-butyl ((6-(2,2′-dichloro-3′-(3-formyl-2-methoxy-4-oxo-4H-pyrido[1,2-a]pyrimidin-8-yl)-[1,1′-biphenyl]-3-yl)-2-methoxypyridin-3-yl)methyl)((5-oxopyrrolidin-2-yl)methyl)carbamate (0.25 g, 0.33 mmol) and (S)-1-aminopropan-2-ol (52 uL, 0.66 mmol) in DCM (2 mL) was added sodium acetate (81 mg, 0.99 mmol) and sodium triacetyloxyboranuide (0.14 g, 0.66 mmol). The mixture was stirred at rt for 2 h under N₂. Trifluoroacetic acid (3.5 mL, 47 mmol) was added to the mixture and stirred at rt for 0.5 h under N₂. The mixture was concentrated to give the residue. The residue was purified by reverse phase HPLC to afford 8-[2-chloro-3-[2-chloro-3-[6-methoxy-5-[[[(2S)-5-oxopyrrolidin-2-yl]methylamino]methyl]-2-pyridyl]phenyl]phenyl]-3-[[[(2S)-2-hydroxypropyl]amino]methyl]-2-methoxy-pyrido[1,2-a]pyrimidin-4-one (35.5 mg, 14% yield, m/z: 717[M+H]⁺, observed) as a yellow solid. ¹H NMR (400 MHz, Methanol-d₄): δ. 9.02 (d, J=7.6 Hz, 1H), 8.35 (s, 1H), 7.71-7.70 (m, 2H), 7.57-7.51 (m, 3H), 7.48-7.32 (m, 3H), 7.33 (dd, J=1.2 Hz, J=7.6 Hz, 1H), 7.20 (d, J=7.2 Hz, 1H), 4.20 (s, 2H), 4.05 (s, 3H), 3.99-3.98 (m, 1H), 3.95 (s, 3H), 3.91-3.90 (m, 2H), 3.83-8.82 (m, 1H), 3.03 (dd, J=12.8 Hz, J=2.8 Hz, 1H), 2.82-2.74 (m, 3H), 2.31-2.20 (m, 3H), 1.79-1.71 (m, 1H), 1.14 (d, J=6.4 Hz).

### Example 348: 8-(2,2′-Dichloro-3′-(6-methoxy-5-(((((S)-5-oxopyrrolidin-2-yl)methyl)amino)methyl)pyridin-2-yl)-[1,1′-biphenyl]-3-yl)-3-((((1r,3r)-3-hydroxy-3-methylcyclobutyl)amino)methyl)-2-methoxy-4H-pyrido[1,2-a]pyrimidin-4-one

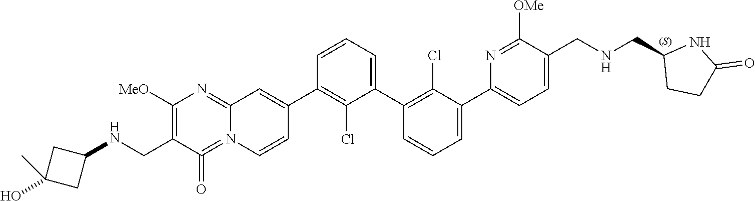

### tert-Butyl ((6-(2,2′-dichloro-3′-(3-((((1r,3r)-3-hydroxy-3-methylcyclobutyl)amino)methyl)-2-methoxy-4-oxo-4H-pyrido[1,2-a]pyrimidin-8-yl)-[1,1′-biphenyl]-3-yl)-2-methoxypyridin-3-yl)methyl)(((S)-5-oxopyrrolidin-2-yl)methyl)carbamate

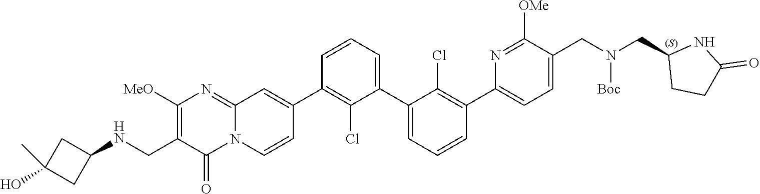

To a mixture of (S)-tert-butyl ((6-(2,2′-dichloro-3′-(3-formyl-2-methoxy-4-oxo-4H-pyrido[1,2-a]pyrimidin-8-yl)-[1,1′-biphenyl]-3-yl)-2-methoxypyridin-3-yl)methyl)((5-oxopyrrolidin-2-yl)methyl)carbamate (0.2 g, 0.26 mmol) and (1r,3r)-3-amino-1-methylcyclobutanol, HCl (73 mg, 0.53 mmol) in DCM/trimethoxymethane mixture (5:1, 2.4 mL) was added sodium acetate (64 mg, 0.79 mmol). The mixture was stirred at rt for 12 h. Sodium triacetyloxyboranuide (0.11 g, 0.53 mmol) was added to the mixture and stirring continued at rt for 2 h. The mixture was filtered and the filtrate was concentrated The residue was purified by normal phase SiO₂ prep-TLC (50% MeOH/EtOAc) to afford tert-butyl ((6-(2,2′-dichloro-3′-(3-((((1r,3r)-3-hydroxy-3-methylcyclobutyl)amino)methyl)-2-methoxy-4-oxo-4H-pyrido[1,2-a]pyrimidin-8-yl)-[1,1′-biphenyl]-3-yl)-2-methoxypyridin-3-yl)methyl)(((S)-5-oxopyrrolidin-2-yl)methyl)carbamate (50 mg) as a white solid (m/z: 843[M+H]⁺, observed). 8-(2,2′-Dichloro-3′-(6-methoxy-5-(((((S)-5-oxopyrrolidin-2-yl)methyl)amino)methyl)pyridin-2-yl)-[1,1′-biphenyl]-3-yl)-3-((((1r,3r)-3-hydroxy-3-methylcyclobutyl)amino)methyl)-2-methoxy-4H-pyrido[1,2-a]pyrimidin-4-one

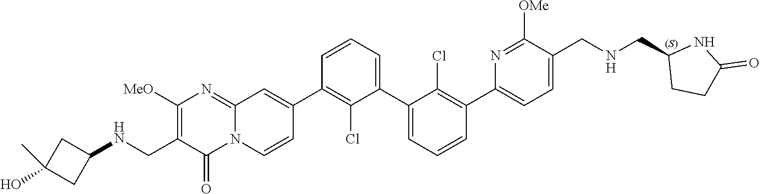

To a solution of tert-butyl ((6-(2,2′-dichloro-3′-(3-((((1r,3r)-3-hydroxy-3-methylcyclobutyl)amino)methyl)-2-methoxy-4-oxo-4H-pyrido[1,2-a]pyrimidin-8-yl)-[1,1′-biphenyl]-3-yl)-2-methoxypyridin-3-yl)methyl)(((S)-5-oxopyrrolidin-2-yl)methyl)carbamate (40 mg, 0.047 mmol) in DCM (3 mL) was added trifluoroacetic acid (0.1 mL, 1.35 mmol). The mixture was stirred at rt for 1 h. The reaction mixture was concentrated and the residue was purified by reverse phase HPLC to afford 8-(2,2′-dichloro-3′-(6-methoxy-5-(((((S)-5-oxopyrrolidin-2-yl)methyl)amino)methyl)pyridin-2-yl)-[1,1′-biphenyl]-3-yl)-3-((((1r,3r)-3-hydroxy-3-methylcyclobutyl)amino)methyl)-2-methoxy-4H-pyrido[1,2-a]pyrimidin-4-one (7.4 mg, 20% yield, m/z: 743 [M+H]⁺ observed). ¹H NMR (400 MHz, Methanol-d₄): δ 9.09 (d, J=7.2 Hz, 1H), 7.76-7.74 (m, 2H), 7.68-7.59 (m, 3H), 7.55-7.49 (m, 3H), 7.41 (d, J=7.2 Hz, 1H), 7.26 (d, J=7.6 Hz, 1H), 4.13 (s, 3H), 4.04 (s, 3H), 3.87-3.80 (m, 5H), 3.59-3.54 (m, 1H), 2.75-2.66 (m, 2H), 2.37-2.27 (m, 5H), 1.92-1.84 (m, 3H), 1.34 (s, 3H).

### Example 227: 2-Chloro-8-(2,2′-dichloro-3′-(6-methoxy-5-(((((S)-5-oxopyrrolidin-2-yl)methyl)amino) methyl)pyridin-2-yl)-[1,1′-biphenyl]-3-yl)-3-(((((S)-5-oxopyrrolidin-2-yl)methyl)amino) methyl)-4H-pyrido[1,2-a]pyrimidin-4-one

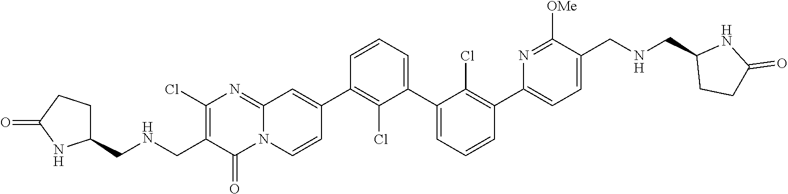 [SMILES: COc1nc(-c2cccc(-c3cccc(-c4ccn5c(=O)c(CNC[C@@H]6CCC(=O)N6)c(Cl)nc5c4)c3Cl)c2Cl)ccc1CNC[C@@H]1CCC(=O)N1]

### Ethyl 8-(3-bromo-2-chlorophenyl)-2-hydroxy-4-oxo-4H-pyrido[1,2-a]pyrimidine-3-carboxylate

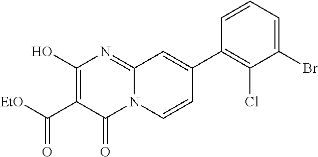 [SMILES: CCOC(=O)c1c(O)nc2cc(-c3cccc(Br)c3Cl)ccn2c1=O]

To a mixture of ethyl 8-bromo-2-hydroxy-4-oxo-4H-pyrido[1,2-a]pyrimidine-3-carboxylate (10 g, 32 mmol) and 2-(3-bromo-2-chlorophenyl)-4,4,5,5-tetramethyl-1,3,2-dioxaborolane (9 g, 29 mmol) in THF/water mixture (10:1, 220 mL) was added 1,1′-bis(diphenylphosphino)ferrocene-palladium(ii) dichloride dichloromethane complex (1.3 g, 1.6 mmol) and potassium carbonate (13 g, 96 mmol) in one portion under N₂ atmosphere. The mixture was stirred at 80° C. for 3 h. The mixture was concentrated to give the residue. The residue was triturated with petroleum ether/ethyl acetate/water mixture (5:5:4 (v/v/v), 140 mL). The mixture was filtered. The filter cake was washed with EtOH (20 mL) and acetonitrile (20 mL) and dried in vacuo. The crude product was triturated with petroleum ether/ethyl acetate/water/EtOH/THF mixture (5:5:4:2:2, 180 mL) to obtain ethyl 8-(3-bromo-2-chlorophenyl)-2-hydroxy-4-oxo-4H-pyrido[1,2-a]pyrimidine-3-carboxylate (6 g, 46% yield) as a brown solid. ¹H NMR (400 MHz, DMSO-d₆): δ 8.52 (d, J=7.2 Hz, 1H), 7.88 (dd, J=8 Hz, J=1.6 Hz, 1H), 7.52 (dd, J=7.6 Hz, J=1.2 Hz, 1H), 7.42 (t, J=8 Hz, 1H), 6.83 (s, 1H), 6.69 (dd, J=7.6 Hz, J=1.6 Hz, 1H), 4.09-4.04 (m, 2H), 1.21 (t, J=7.2 Hz, 3H).

### Ethyl 8-(3-bromo-2-chlorophenyl)-2-chloro-4-oxo-4H-pyrido[1,2-a]pyrimidine-3-carboxylate

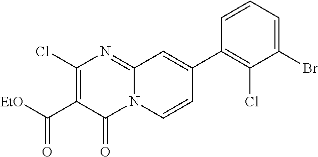 [SMILES: CCOC(=O)c1c(Cl)nc2cc(-c3cccc(Br)c3Cl)ccn2c1=O]

To a mixture of ethyl 8-(3-bromo-2-chlorophenyl)-2-hydroxy-4-oxo-4H-pyrido[1,2-a]pyrimidine-3-carboxylate (5 g, 12 mmol) in ClCH₂CH₂Cl (50 mL) was added oxalyl chloride (5 mL, 59 mmol) slowly followed by dropwise addition of DMF (0.5 mL, 5.9 mmol). The mixture was stirred at 60° C. for 4 h under N₂ atmosphere. The reaction was quenched with water (40 mL). The mixture was extracted with ethyl acetate/THF mixture (1:1, 200 mL). The organic phase was dried with anhydrous sodium sulfate, filtered and concentrated. The residue was purified by normal phase SiO₂ chromatography (0-30% [50% THF in ethyl acetate]/petroleum ether) to afford ethyl 8-(3-bromo-2-chlorophenyl)-2-chloro-4-oxo-4H-pyrido[1,2-a]pyrimidine-3-carboxylate as a yellow gum (1.1 g, 11% yield, m/z: 443 [M+H]⁺ observed). ¹H NMR (400 MHz, DMSO-d₆): δ 9.04 (d, J=7.2 Hz, 1H), 7.97 (d, J=8 Hz, 1H), 7.92-7.91 (m, 1H), 7.70-7.68 (m, 1H), 7.60-7.58 (m, 1H), 7.48 (t, J=8 Hz, 1H), 4.35 (q, J=7.2 Hz, 2H), 1.32 (t, J=7.2 Hz, 3H).

### 8-(3-Bromo-2-chlorophenyl)-2-chloro-3-(hydroxymethyl)-4H-pyrido[1,2-a]pyrimidin-4-one

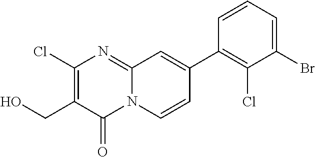 [SMILES: O=c1c(CO)c(Cl)nc2cc(-c3cccc(Br)c3Cl)ccn12]

To a mixture of ethyl 8-(3-bromo-2-chlorophenyl)-2-chloro-4-oxo-4H-pyrido[1,2-a]pyrimidine-3-carboxylate (1.12 g, 2.53 mmol) in CH₂Cl₂ (15 mL) was added diisobutyl aluminium hydride (1 M, 5 mL) dropwise at −20° C. under N₂ atmosphere. The mixture was stirred at rt for 4 h. The reaction was quenched with dropwise addition of water (0.4 mL) at 0° C. followed by THF (100 mL). The mixture was dried with anhydrous sodium sulfate, filtered and concentrated. The residue was purified by normal phase SiO₂ chromatography (0-35% 50% THF in ethyl acetate/petroleum ether) to afford 8-(3-bromo-2-chlorophenyl)-2-chloro-3-(hydroxymethyl)-4H-pyrido[1,2-a]pyrimidin-4-one (0.29 g, 26% yield) as a yellow solid. ¹H NMR (400 MHz, DMSO-d₆): δ 9.01 (d, J=7.2 Hz, 1H), 7.96 (dd, J=8 Hz, J=1.2 Hz, 1H), 7.80 (d, J=1.6 Hz, 1H), 7.61-7.56 (m, 2H), 7.48 (t, J=8 Hz, 1H), 5.02 (t, J=5.2 Hz, 1H), 4.58 (d, J=5.2 Hz, 2H).

### 6-(2,2′-Dichloro-3′-(2-chloro-3-(hydroxymethyl)-4-oxo-4H-pyrido[1,2-a]pyrimidin-8-yl)-[1,1′-biphenyl]-3-yl)-2-methoxynicotinaldehyde

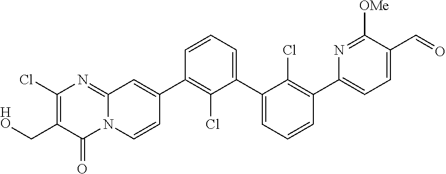 [SMILES: COc1nc(-c2cccc(-c3cccc(-c4ccn5c(=O)c(CO)c(Cl)nc5c4)c3Cl)c2Cl)ccc1C=O]

To a mixture of 8-(3-bromo-2-chlorophenyl)-2-chloro-3-(hydroxymethyl)-4H-pyrido[1,2-a]pyrimidin-4-one (0.22 g, 0.55 mmol) and 6-(2-chloro-3-(4,4,5,5-tetramethyl-1,3,2-dioxaborolan-2-yl)phenyl)-2-methoxynicotinaldehyde (0.22 g, 0.58 mmol) in 1,4-dioxane/water mixture (5:1, 6 mL) was added [1,1′-bis(di-tert-butylphosphino)ferrocene]dichloropalladium(II) (0.02 g, 0.03 mmol) and potassium phosphate (0.35 g, 1.65 mmol) in one portion under N₂ atmosphere. The mixture was stirred at 110° C. for 0.5 h. The mixture was concentrated, and the residue suspended in water (5 mL) and brine (5 mL). The mixture was extracted with ethyl acetate/THF mixture (1:1, 50 mL). The organic phase was dried with anhydrous sodium sulfate, filtered and concentrated. The residue was purified by normal phase SiO₂ chromatography (0-50% 50% THE in ethyl acetate/petroleum ether) to afford 6-(2,2′-dichloro-3′-(2-chloro-3-(hydroxymethyl)-4-oxo-4H-pyrido[1,2-a]pyrimidin-8-yl)-[1,1′-biphenyl]-3-yl)-2-methoxynicotinaldehyde (150 mg, 39% yield) as a light-yellow solid. ¹H NMR (400 MHz, DMSO-d₆): δ 10.31 (s, 1H), 9.03 (d, J=7.2 Hz, 1H), 8.23 (d, J=8 Hz, 1H), 7.84 (m, 1H), 7.78-7.77 (m, 1H), 7.71-7.56 (m, 6H), 7.48 (t, J=7.6 Hz, 1H), 5.03 (t, J=6.4 Hz, 1H), 4.58 (d, J=6.4 Hz, 2H), 4.07 (s, 3H).

### 2-Chloro-8-(2,2′-dichloro-3′-(5-formyl-6-methoxypyridin-2-yl)-[1,1′-biphenyl]-3-yl)-4-oxo-4H-pyrido[1,2-a]pyrimidine-3-carbaldehyde

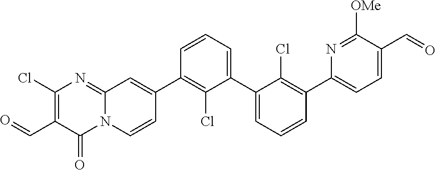 [SMILES: COc1nc(-c2cccc(-c3cccc(-c4ccn5c(=O)c(C=O)c(Cl)nc5c4)c3Cl)c2Cl)ccc1C=O]

To a mixture of 6-(2,2′-dichloro-3′-(2-chloro-3-(hydroxymethyl)-4-oxo-4H-pyrido[1,2-a]pyrimidin-8-yl)-[1,1′-biphenyl]-3-yl)-2-methoxynicotinaldehyde (140 mg, 0.25 mmol) in CHCl₃ (6 mL) was added manganese(IV) oxide (172 mg, 1.98 mmol) in one portion under N₂ atmosphere. The mixture was stirred at rt for 16 h. To the mixture was added manganese(IV) oxide (214 mg, 2.47 mmol). The mixture was stirred at 45° C. for 4 h. The mixture was filtered. The filtrate was concentrated to afford crude 2-chloro-8-(2,2′-dichloro-3′-(5-formyl-6-methoxypyridin-2-yl)-[1,1′-biphenyl]-3-yl)-4-oxo-4H-pyrido[1,2-a]pyrimidine-3-carbaldehyde (85 mg, 38% yield, m/z: 564 [M+H]⁺, 566 [M+2+H]⁺ observed) as a yellow solid. ¹H NMR (400 MHz, DMSO-d₆): δ 10.28 (s, 1H), 10.25 (s, 1H), 9.19 (d, J=7.2 Hz, 1H), 8.20 (d, J=7.6 Hz, 1H), 8.00 (s, 1H), 7.80-7.79 (m, 1H), 7.76-7.74 (m, 1H), 7.68-7.59 (m, 4H), 7.56-7.53 (m, 1H), 7.47 (d, J=7.6 Hz, 1H), 4.05 (s, 3H).

### 2-Chloro-8-(2,2′-dichloro-3′-(6-methoxy-5-(((((S)-5-oxopyrrolidin-2-yl)methyl)amino) methyl)pyridin-2-yl)-[1,1′-biphenyl]-3-yl)-3-(((((S)-5-oxopyrrolidin-2-yl)methyl)amino) methyl)-4H-pyrido[1,2-a]pyrimidin-4-one

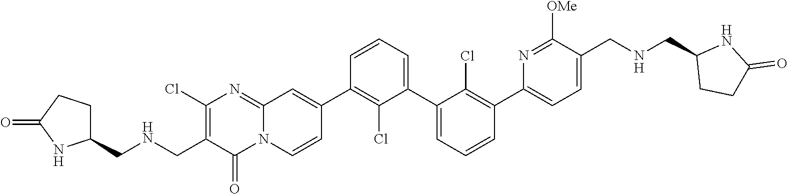 [SMILES: COc1nc(-c2cccc(-c3cccc(-c4ccn5c(=O)c(CNC[C@@H]6CCC(=O)N6)c(Cl)nc5c4)c3Cl)c2Cl)ccc1CNC[C@@H]1CCC(=O)N1]

To a mixture of 2-chloro-8-(2,2′-dichloro-3′-(5-formyl-6-methoxypyridin-2-yl)-[1,1′-biphenyl]-3-yl)-4-oxo-4H-pyrido[1,2-a]pyrimidine-3-carbaldehyde (75 mg, 0.13 mmol) and (S)-5-(aminomethyl)-2-pyrrolidinone, HCl (70 mg, 0.46 mmol) in CH₂Cl₂ (3 mL) was added sodium acetate (11 mg, 0.13 mmol) and sodium triacetoxyborohydride (84 mg, 0.4 mmol) in one portion under N₂ atmosphere. The mixture was stirred at rt for 12 h. The mixture was concentrated. The residue was purified by reverse phase HPLC to afford 2-chloro-8-(2,2′-dichloro-3′-(6-methoxy-5-(((((S)-5-oxopyrrolidin-2-yl)methyl)amino)methyl)pyridin-2-yl)-[1,1′-biphenyl]-3-yl)-3-(((((S)-5-oxopyrrolidin-2-yl)methyl)amino)methyl)-4H-pyrido[1,2-a]pyrimidin-4-one (18.4 mg, 16% yield, m/z: 760 [M+H]⁺, 762 [M+2+H]⁺ observed) as a yellow solid. ¹H NMR (400 MHz, DMSO-d₆): δ 9.01 (d, J=7.2 Hz, 1H), 8.17 (s, 1H), 7.85-7.82 (m, 2H), 7.71-7.57 (m, 7H), 7.49-7.47 (m, 1H), 7.29 (d, J=7.6 Hz, 1H), 3.93 (s, 3H), 3.87 (s, 2H), 3.73-3.65 (m, 2H), 3.63-3.58 (m, 2H), 2.58-2.54 (m, 4H), 2.15-2.05 (m, 6H), 1.76-1.65 (m, 2H).

### Example 212: 7-(2,2′-Dichloro-3′-(6-methoxy-5-(((((S)-5-oxopyrrolidin-2-yl)methyl)amino)methyl)pyridin-2-yl)-[1,1′-biphenyl]-3-yl)-2-(((((S)-5-oxopyrrolidin-2-yl)methyl)amino)methyl)-4H-pyrido[1,2-a]pyrimidin-4-one

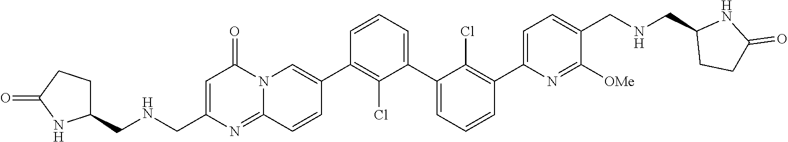 [SMILES: COc1nc(-c2cccc(-c3cccc(-c4ccc5nc(CNC[C@@H]6CCC(=O)N6)cc(=O)n5c4)c3Cl)c2Cl)ccc1CNC[C@@H]1CCC(=O)N1]

### 7-Bromo-2-(chloromethyl)-4H-pyrido[1,2-a]pyrimidin-4-one

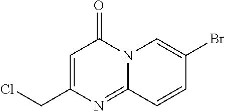 [SMILES: O=c1cc(CCl)nc2ccc(Br)cn12]

A mixture of 5-bromopyridin-2-amine (5 g, 28.9 mmol) and ethyl 4-chloro-3-oxo-butanoate (6.66 g, 40.4 mmol) in polyphosphoric acid (10 mL) was degassed with nitrogen gas and stirred at 110° C. for 0.5 h under N₂ atmosphere. The reaction mixture was added to water (350 mL), and extracted with EtOAc (3×150 ml). The combined organic layers were washed with brine (50 mL), dried over anhydrous sodium sulfate, and filtered to give 7-bromo-2-(chloromethyl)-4H-pyrido[1,2-a]pyrimidin-4-one was obtained as brown solid (6.5 g, crude). ¹H NMR (400 MHz, DMSO-d₆): δ 8.96 (d, J=2 Hz, 1H), 8.08 (dd, J=2.4 Hz, J=9.6 Hz, 1H), 7.62 (d, J=9.2 Hz, 1H), 6.58 (s, 1H), 4.57 (s, 2H).

### (S)-tert-Butyl ((7-bromo-4-oxo-4H-pyrido[1,2-a]pyrimidin-2-yl)methyl)((5-oxopyrrolidin-2-yl)methyl)carbamate

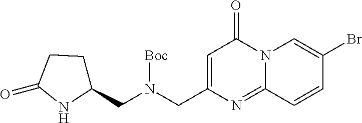 [SMILES: CC(C)(C)OC(=O)N(Cc1cc(=O)n2cc(Br)ccc2n1)C[C@@H]1CCC(=O)N1]

A mixture of 7-bromo-2-(chloromethyl)-4H-pyrido[1,2-a]pyrimidin-4-one (2 g, 7.3 mmol), (5S)-5-(aminomethyl)pyrrolidin-2-one, HCl (1.4 g, 9.5 mmol) and K₂CO₃ (5.0 g, 36.5 mmol) in acetonitrile (30 mL) was degassed with nitrogen gas and stirred at 80° C. for 12 h under N₂ atmosphere. Di-tert-butyl dicarbonate (14.6 mmol, 3.3 mL) in THE (10 mL) was added and the mixture was stirred at 20° C. for 1 h under N₂ atmosphere. The reaction mixture was added to water (30 mL) and extracted with EtOAc (3×30 ml). The combined organic layers were washed with brine (20 mL), dried over anhydrous sodium sulfate, filtered and concentrated under reduced pressure. The residue was purified by normal phase SiO₂ chromatography (0-6% EtOAc/petroleum ether) to afford (S)-tert-butyl ((7-bromo-4-oxo-4H-pyrido[1,2-a]pyrimidin-2-yl)methyl)((5-oxopyrrolidin-2-yl)methyl)carbamate as yellow oil (1.2 g, 26% yield). ¹H NMR (400 MHz, DMSO-d₆): δ 8.99 (d, J=5.6 Hz, 1H), 8.09 (d, J=9.2 Hz, 1H), 7.84 (m, 1H), 7.59 (d, J=9.6 Hz, 1H), 6.19 (d, J=18.4 Hz, 1H), 4.46-4.34 (m, 2H), 3.79-3.71 (m, 2H), 3.34-2.91 (m, 2H), 1.72-1.64 (m, 3H), 1.43-1.40 (m, 9H).

### (S)-tert-Butyl ((7-(2,2′-dichloro-3′-(5-formyl-6-methoxypyridin-2-yl)-[1,1′-biphenyl]-3-yl)-4-oxo-4H-pyrido[1,2-a]pyrimidin-2-yl)methyl)((5-oxopyrrolidin-2-yl)methyl)carbamate

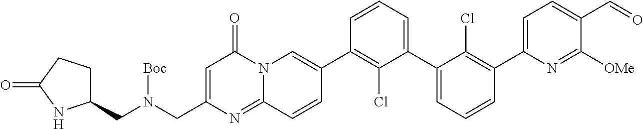 [SMILES: COc1nc(-c2cccc(-c3cccc(-c4ccc5nc(CN(C[C@@H]6CCC(=O)N6)C(=O)OC(C)(C)C)cc(=O)n5c4)c3Cl)c2Cl)ccc1C=O]

A mixture of tert-butyl (S)-tert-butyl ((7-bromo-4-oxo-4H-pyrido[1,2-a]pyrimidin-2-yl)methyl)((5-oxopyrrolidin-2-yl)methyl)carbamate (0.29 g, 0.64 mmol), 6-(2,2′-dichloro-3′-(4,4,5,5-tetramethyl-1,3,2-dioxaborolan-2-yl)-[1,1′-biphenyl]-3-yl)-2-methoxynicotinaldehyde (0.34 g, 0.7 mmol), potassium phosphate (0.41 g, 1.93 mmol) and di-tert-butyl(cyclopentyl)phosphane;dichloropalladium;iron (0.08 g, 0.12 mmol) in dioxane (1 mL) and water (0.2 mL) was degassed with nitrogen gas and stirred at 80° C. for 2 h under N₂ atmosphere. The reaction mixture was added to water (30 mL) and extracted with EtOAc (3×30 ml). The combined organic layers were washed with brine (20 mL), dried over anhydrous sodium sulfate, filtered and concentrated under reduced pressure. The residue was purified by normal phase SiO₂ chromatography (0-20% EtOAc/petroleum ether) to afford (S)-tert-butyl ((7-(2,2′-dichloro-3′-(5-formyl-6-methoxypyridin-2-yl)-[1,1′-biphenyl]-3-yl)-4-oxo-4H-pyrido[1,2-a]pyrimidin-2-yl)methyl)((5-oxopyrrolidin-2-yl)methyl)carbamate as yellow oil (0.4 g, 85% yield, m/z: 728.1 [M+H]⁺ observed).

### tert-Butyl ((7-(2,2′-dichloro-3′-(6-methoxy-5-(((((S)-5-oxopyrrolidin-2-yl)methyl)amino) methyl)pyridin-2-yl)-[1,1′-biphenyl]-3-yl)-4-oxo-4H-pyrido[1,2-a]pyrimidin-2-yl)methyl)(((S)-5-oxopyrrolidin-2-yl)methyl)carbamate

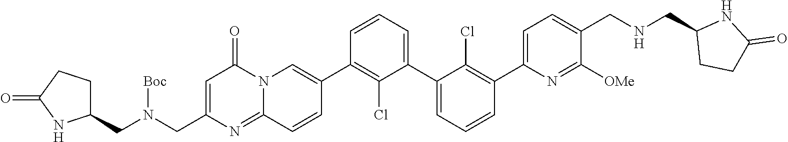 [SMILES: COc1nc(-c2cccc(-c3cccc(-c4ccc5nc(CN(C[C@@H]6CCC(=O)N6)C(=O)OC(C)(C)C)cc(=O)n5c4)c3Cl)c2Cl)ccc1CNC[C@@H]1CCC(=O)N1]

A mixture of (S)-tert-butyl ((7-(2,2′-dichloro-3′-(5-formyl-6-methoxypyridin-2-yl)-[1,1′-biphenyl]-3-yl)-4-oxo-4H-pyrido[1,2-a]pyrimidin-2-yl)methyl)((5-oxopyrrolidin-2-yl)methyl)carbamate (0.35 g, 0.48 mmol), (S)-5-(aminomethyl)pyrrolidin-2-one, HCl (0.43 g, 2.88 mmol), and sodium acetate (0.23 g, 2.88 mmol) in DCM/MeOH (3:1, 8 mL) was degassed with nitrogen gas and stirred at 25° C. for 0.5 h. Sodium cyanoborohydride (0.18 g, 2.88 mmol) was added and the reaction mixture was stirred at 25° C. for 0.5 h. The solvent was evaporated to afford crude tert-butyl ((7-(2,2′-dichloro-3′-(6-methoxy-5-(((((S)-5-oxopyrrolidin-2-yl)methyl)amino)methyl)pyridin-2-yl)-[1,1′-biphenyl]-3-yl)-4-oxo-4H-pyrido[1,2-a]pyrimidin-2-yl)methyl)(((S)-5-oxopyrrolidin-2-yl)methyl)carbamate.

### 7-(2,2′-Dichloro-3′-(6-methoxy-5-(((((S)-5-oxopyrrolidin-2-yl)methyl)amino)methyl)pyridin-2-yl)-[1,1′-biphenyl]-3-yl)-2-(((((S)-5-oxopyrrolidin-2-yl)methyl)amino)methyl)-4H-pyrido[1,2-a]pyrimidin-4-one

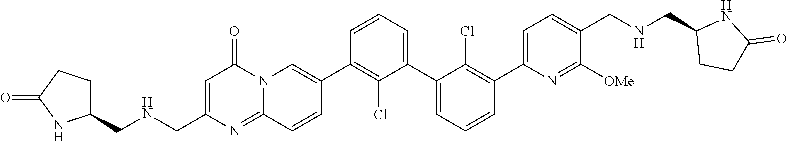 [SMILES: COc1nc(-c2cccc(-c3cccc(-c4ccc5nc(CNC[C@@H]6CCC(=O)N6)cc(=O)n5c4)c3Cl)c2Cl)ccc1CNC[C@@H]1CCC(=O)N1]

To a solution of crude tert-butyl ((7-(2,2′-dichloro-3′-(6-methoxy-5-(((((S)-5-oxopyrrolidin-2-yl)methyl)amino)methyl)pyridin-2-yl)-[1,1′-biphenyl]-3-yl)-4-oxo-4H-pyrido[1,2-a]pyrimidin-2-yl)methyl)(((S)-5-oxopyrrolidin-2-yl)methyl)carbamate in DCM/MeOH (3:1, 8 mL), trifluoroacetic acid (2 mL) in DCM (6 mL) was added and the mixture was stirred at 20° C. for 1 h. The reaction mixture was filtered and the filtrate was concentrated. The residue was purified by reverse phase HPLC to afford 7-(2,2′-dichloro-3′-(6-methoxy-5-(((((S)-5-oxopyrrolidin-2-yl)methyl)amino)methyl)pyridin-2-yl)-[1,1′-biphenyl]-3-yl)-2-(((((S)-5-oxopyrrolidin-2-yl)methyl)amino)methyl)-4H-pyrido[1,2-a]pyrimidin-4-one as a white solid. (58.8 mg, 15% yield, m/z:726.2 [M+H]⁺ observed). ¹H NMR (400 MHz, Methanol-d₄): δ 9.14 (d, J=1.2 Hz, 1H), 8.18 (dd, J=2 Hz, J=8.8 Hz, 1H), 7.85 (m, 2H), 7.70-7.44 (m, 6H), 7.36 (d, J=7.6 Hz, 1H), 6.61 (s, 1H), 4.25-3.97 (m, 9H), 3.15-2.93 (m, 4H), 2.46-2.36 (m, 6H), 1.98-1.91 (m, 2H).

### Example 324: 7-(2,2′-Dichloro-3′-(6-methoxy-5-(((((S)-5-oxopyrrolidin-2-yl)methyl)amino)methyl)pyridin-2-yl)-[1,1′-biphenyl]-3-yl)-2-((((S)-2-hydroxypropyl)amino)methyl)-4H-pyrido[1,2-a]pyrimidin-4-one

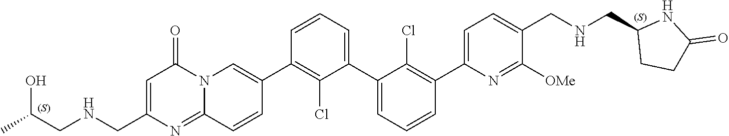

### (S)-tert-butyl((6-(2,2′-Dichloro-3′-(2-(hydroxymethyl)-4-oxo-4H-pyrido[1,2-a]pyrimidin-7-yl)-[1,1′-biphenyl]-3-yl)-2-methoxypyridin-3-yl)methyl)((5-oxopyrrolidin-2-yl)methyl)carbamate

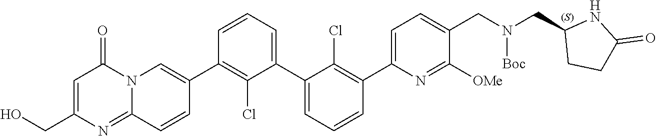

A mixture of 7-bromo-2-(hydroxymethyl)pyrido[1,2-a]pyrimidin-4-one (0.44 g, 1.7 mmol), tert-butyl N-[[6-[2-chloro-3-[2-chloro-3-(4,4,5,5-tetramethyl-1,3,2-dioxaborolan-2-yl)phenyl]phenyl]-2-methoxy-3-pyridyl]methyl]-N-[[(2R)-5-oxopyrrolidin-2-yl]methyl]carbamate (1.2 g, 1.7 mmol), [1,1′-Bis(di-tert-butylphosphino)ferrocene]dichloropalladium(II) (60 mg, 87 umol) and potassium phosphate (1.1 g, 5.3 mmol) in dioxane/water mixture (4:1, 25 mL) was stirred at 110° C. for 0.5 h under N₂. The reaction mixture was combined with another batch at 0.5 g scale. The combined mixture was concentrated and the residue was purified by normal phase SiO₂ chromatography (0-85% EtOAc/petroleum ether) to afford (S)-tert-butyl((6-(2,2′-dichloro-3′-(2-(hydroxymethyl)-4-oxo-4H-pyrido[1,2-a]pyrimidin-7-yl)-[1,1′-biphenyl]-3-yl)-2-methoxypyridin-3-yl)methyl)((5-oxopyrrolidin-2-yl)methyl)carbamate as a yellow solid (0.55 g, 32% yield, m/z: 730 [M+H]⁺, observed).

### (S)-tert-Butyl((6-(2,2′-dichloro-3′-(2-(hydroxymethyl)-4-oxo-4H-pyrido[1,2-a]pyrimidin-7-yl)-[1,1′-biphenyl]-3-yl)-2-methoxypyridin-3-yl)methyl)((5-oxopyrrolidin-2-yl)methyl)carbamate

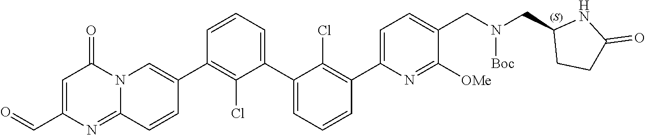

A mixture of tert-butyl N-[[6-[2-chloro-3-[2-chloro-3-[2-(hydroxymethyl)-4-oxo-pyrido[1,2-a]pyrimidin-7-yl]phenyl]phenyl]-2-methoxy-3-pyridyl]methyl]-N-[[(2S)-5-oxopyrrolidin-2-yl]methyl]carbamate (0.49 g, 0.67 mmol) and Dess-Martin periodinane (0.57 g, 1.34 mmol) in DCM (10 mL) was stirred at rt for 2 h under N₂. The reaction mixture was concentrated, and the residue was washed with EtOAc/water mixture (1:1, 200 mL). The combined organic phase was dried with anhydrous sodium sulfate, filtered and concentrated. The residue was purified by normal phase SiO₂ chromatography (0-45% EtOAc/petroleum ether) to afford (S)-tert-butyl((6-(2,2′-dichloro-3′-(2-(hydroxymethyl)-4-oxo-4H-pyrido[1,2-a]pyrimidin-7-yl)-[1,1′-biphenyl]-3-yl)-2-methoxypyridin-3-yl)methyl)((5-oxopyrrolidin-2-yl)methyl)carbamate as a yellow solid. (0.15 g, 31% yield, m/z: 728 [M+H]⁺).

### tert-Butyl((6-(2,2′-dichloro-3′-(2-((((S)-2-hydroxypropyl)amino)methyl)-4-oxo-4H-pyrido[1,2-a]pyrimidin-7-yl)-[1,1′-biphenyl]-3-yl)-2-methoxypyridin-3-yl)methyl)(((S)-5-oxopyrrolidin-2-yl)methyl)carbamate

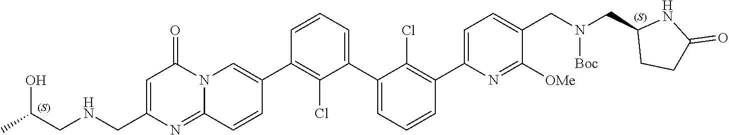

A mixture of tert-buty1N-[[6-[2-chloro-3-[2-chloro-3-(2-formyl-4-oxo-pyrido[1,2-a]pyrimidin-7-yl)phenyl]phenyl]-2-methoxy-3-pyridyl]methyl]-N-[[(2S)-5-oxopyrrolidin-2-yl]methyl]carbamate (88 mg, 0.12 mmol), (2S)-1-aminopropan-2-ol (36 mg, 0.5 mmol), sodium acetate (50 mg, 0.6 mmol) in DCM/MeOH (1:1, 4 mL) was stirred at rt for 12 h under N₂. Sodium cyanoborohydride (15 mg, 0.25 mmol) was added and the mixture was stirred at rt for 3 h under N₂. The solvent was evaporated to afford crude tert-butyl N-[[6-[2-chloro-3-[2-chloro-3-[2-[[[(2S)-2-hydroxypropyl]amino]methyl]-4-oxo-pyrido[1,2-a]pyrimidin-7-yl]phenyl]phenyl]-2-methoxy-3-pyridyl]methyl]-N-[[(2S)-5-oxopyrrolidin-2-yl]methyl]carbamate. The crude product was used directly without further purification. (m/z: 787 [M+H]⁺).

### 7-(2,2′-Dichloro-3′-(6-methoxy-5-(((((S)-5-oxopyrrolidin-2-yl)methyl)amino)methyl)pyridin-2-yl)-[1,1′-biphenyl]-3-yl)-2-((((S)-2-hydroxypropyl)amino)methyl)-4H-pyrido[1,2-a]pyrimidin-4-one

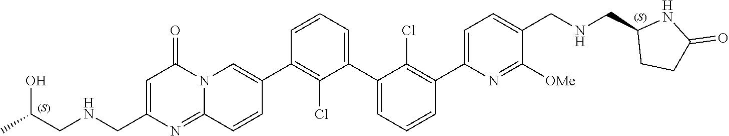

To a solution of crude N-[[6-[2-chloro-3-[2-chloro-3-[2-[[[(2S)-2-hydroxypropyl]amino]methyl]-4-oxo-pyrido[1,2-a]pyrimidin-7-yl]phenyl]phenyl]-2-methoxy-3-pyridyl]methyl]-N-[[(2S)-5-oxopyrrolidin-2-yl]methyl]carbamate (0.3 g, 0.4 mmol) in DCM (2 mL) was added trifluoroacetic acid (15 mL, 0.5 mol) in DCM (4 mL). The mixture was stirred at rt for 1 h under N₂. The reaction mixture was concentrated and the residue was purified by reverse phase HPLC to afford 7-(2,2′-dichloro-3′-(6-methoxy-5-(((((S)-5-oxopyrrolidin-2-yl)methyl)amino)methyl) pyridin-2-yl)-[1,1′-biphenyl]-3-yl)-2-((((S)-2-hydroxypropyl)amino)methyl)-4H-pyrido[1,2-a]pyrimidin-4-one as a white solid (12.5 mg, 4% yield), m/z: 687[M+H]⁺). ¹H NMR (400 MHz, Methanol-d₄): δ9.17 (s, 1H), 8.12 (dd, J=9.2 Hz, J=2 Hz, 1H), 7.88 (d, J=9.6 Hz, 1H), 7.80 (d, J=6.8 Hz, 1H), 7.69-7.62 (m, 3H), 7.56-7.52 (m, 2H), 7.43 (dd, J=9.2 Hz, J=2 Hz, 1H), 7.31 (d, J=7.6 Hz, 1H), 6.57 (s, 1H), 4.30 (s, 2H), 4.12-3.91 (m, 7H), 3.13 (s, 1H), 2.98-2.87 (m, 3H), 2.40-2.33 (m, 3H), 1.87-1.86 (m, 1H), 1.27 (d, J=6 Hz, 3H).

### Example 325: 7-(2,2′-dichloro-3′-(6-methoxy-5-(((((S)-5-oxopyrrolidin-2-yl)methyl)amino)methyl) pyridin-2-yl)-[1,1′-biphenyl]-3-yl)-2-((((1r,3r)-3-hydroxy-3-methylcyclobutyl)amino) methyl)-4H-pyrido[1,2-a]pyrimidin-4-one

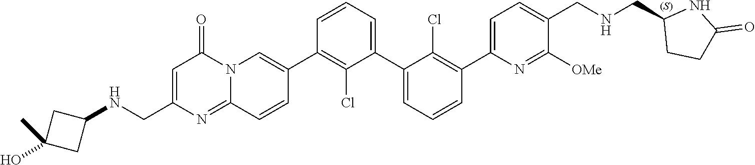

### 7-Bromo-2-((((1r,3r)-3-hydroxy-3-methylcyclobutyl)amino)methyl)-4H-pyrido[1,2-a]pyrimidin-4-one

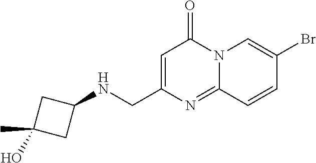 [SMILES: C[C@]1(O)C[C@H](NCc2cc(=O)n3cc(Br)ccc3n2)C1]

A mixture of 7-bromo-4-oxo-pyrido[1,2-a]pyrimidine-2-carbaldehyde (100 mg, 0.4 mmol), 3-amino-1-methyl-cyclobutanol, HCl (70 mg, 0.5 mmol) and sodium acetate (0.1 g, 1 mmol) in DCM/MeOH (3:1.12 mL) was stirred at rt for 12 h under N₂. Sodium cyanoborohydride (50 mg, 0.8 mmol) was added to the mixture and stirred at rt for 3 h under N₂. The solvent was evaporated to afford crude 7-bromo-2-((((1r,3r)-3-hydroxy-3-methylcyclobutyl)amino)methyl)-4H-pyrido[1,2-a]pyrimidin-4-one. The crude product was used directly without further purification. m/z: 338 [(M-t-Butyl)+H]⁺.

### tert-Butyl ((7-bromo-4-oxo-4H-pyrido[1,2-a]pyrimidin-2-yl)methyl)((1r,3r)-3-hydroxy-3-methylcyclobutyl)carbamate

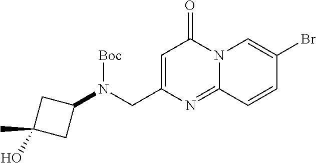

To a solution of crude 7-bromo-2-[[(3-hydroxy-3-methyl-cyclobutyl)amino]methyl]pyrido[1,2-a]pyrimidin-4-one in solvent DCM (8 mL) was added di-tert-butyl dicarbonate (0.3 mL, 1.42 mmol), triethylamine (0.1 mL, 0.89 mmol) was stirred at rt for 1 h under N₂. The reaction mixture was concentrated, and the residue was purified by SiO₂ chromatography (0-45% EtOAc/petroleum ether) to afford tert-butyl ((7-bromo-4-oxo-4H-pyrido[1,2-a]pyrimidin-2-yl)methyl)((1r,3r)-3-hydroxy-3-methylcyclobutyl)carbamate (0.15 g, 96% yield) as a yellow solid. ¹H NMR (400 MHz, CDCl₃): δ9.09 (s, 1H), 7.69 (dd, J=2 Hz, J=9.2 Hz, 1H), 7.40 (d, J=9.6 Hz 1H), 7.20 (s, 1H), 6.27 (s, 1H), 4.26 (br s, 2H), 4.05 (q, J=7.2 Hz, 1H), 2.67-2.22 (m, 2H), 2.08-2.02 (m, 2H), 1.39-1.27 (m, 9H).

### tert-Butyl ((6-(3′-(2-(((tert-butoxycarbonyl)((1r,3r)-3-hydroxy-3-methylcyclobutyl)amino) methyl)-4-oxo-4H-pyrido[1,2-a]pyrimidin-7-yl)-2,2′-dichloro-[1,1′-biphenyl]-3-yl)-2-methoxypyridin-3-yl)methyl)(((S)-5-oxopyrrolidin-2-yl)methyl)carbamate

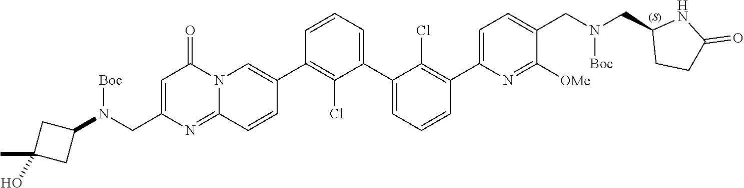

A mixture of tert-butyl N-[[6-[2-chloro-3-[2-chloro-3-(4,4,5,5-tetramethyl-1,3,2-dioxaborolan-2-yl)phenyl]phenyl]-2-methoxy-3-pyridyl]methyl]-N-[[(2S)-5-oxopyrrolidin-2-yl]methyl] carbamate (260 mg, 0.4 mmol), tert-butyl N-[(7-bromo-4-oxo-pyrido[1,2-a]pyrimidin-2-yl)methyl]-N-(3-hydroxy-3-methyl-cyclobutyl)carbamate (0.14 g, 0.3 mmol), potassium phosphate (0.2 g, 1 mmol), ditert-butyl(cyclopentyl)phosphane;dichloropalladium;iron (21 mg, 0.03 mmol) in dioxane/water (5:1, 12 mL) was stirred at 110° C. for 3 h under N₂. The reaction mixture was concentrated and the residue was purified by SiO₂ chromatography (0-75% EtOAc/petroleum ether) to afford tert-butyl ((6-(3′-(2-(((tert-butoxycarbonyl)((1r,3r)-3-hydroxy-3-methylcyclobutyl)amino)methyl)-4-oxo-4H-pyrido[1,2-a]pyrimidin-7-yl)-2,2′-dichloro-[1,1′-biphenyl]-3-yl)-2-methoxypyridin-3-yl)methyl)(((S)-5-oxopyrrolidin-2-yl)methyl)carbamate as a brown solid (0.1 g, 36% yield), m/z: 913[M+H]⁺, observed).

### 7-(2,2′-Dichloro-3′-(6-methoxy-5-(((((S)-5-oxopyrrolidin-2-yl)methyl)amino)methyl)pyridin-2-yl)-[1,1′-biphenyl]-3-yl)-2-((((1r,3r)-3-hydroxy-3-methylcyclobutyl)amino)methyl)-4H-pyrido[1,2-a]pyrimidin-4-one

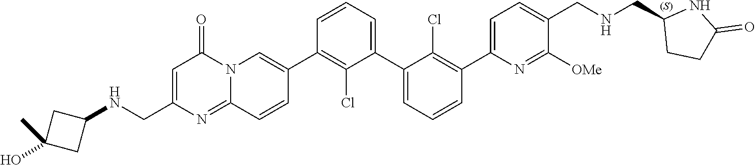

A mixture of tert-butyl N-[[6-[3-[3-[2-[[tert-butoxycarbonyl-(3-hydroxy-3-methyl-cyclobutyl)amino]methyl]-4-oxo-pyrido[1,2-a]pyrimidin-7-yl]-2-chloro-phenyl]-2-chloro-phenyl]-2-methoxy-3-pyridyl]methyl]-N-[[(2S)-5-oxopyrrolidin-2-yl]methyl]carbamate (0.1 g, 0.1 mmol) and trifluoroacetic acid (5 mL, 70 mmol) in DCM (4 mL) was stirred at rt for 0.5 h. The reaction mixture was concentrated and the residue was purified by prep-HPLC to afford 7-(2,2′-dichloro-3′-(6-methoxy-5-(((((S)-5-oxopyrrolidin-2-yl)methyl)amino)methyl)pyridin-2-yl)-[1,1′-biphenyl]-3-yl)-2-((((1r,3r)-3-hydroxy-3-methylcyclobutyl)amino)methyl)-4H-pyrido[1,2-a]pyrimidin-4-one (10 mg, 15% yield), m/z: 713[M+H]⁺, observed. ¹H NMR (400 MHz, DMSO-d₆): δ8.98 (d, J=1.6 Hz, 1H), 8.19 (s, 1H), 8.12 (dd, J=9.2 Hz, J=2 Hz, 1H), 7.85 (d, J=7.2 Hz, 1H), 7.73 (d, J=13.6 Hz, 1H), 7.70-7.69 (m, 3H), 7.64 (t, J=9.6 Hz, 1H), 7.59-7.49 (m, 2H), 7.50 (dd, J=7.2 Hz, J=2 Hz, 1H), 7.32 (d, J=7.2 Hz, 1H), 6.57 (s, 1H), 4.71 (br s, 1H), 3.95 (s, 3H), 3.75 (s, 2H), 3.68 (s, 2H), 2.59 (d, J=6 Hz, 2H), 2.55-2.53 (m, 2H), 2.18-2.12 (m, 5H), 1.80-1.72 (m, 3H), 1.30 (s, 3H).), 2.20-2.14 (m, 2H), 1.89-1.84 (m, 1H), 1.42 (s, 3H).

### Example 288: 7-(2,2′-Dichloro-3′-(6-methoxy-5-(((((S)-5-oxopyrrolidin-2-yl)methyl)amino)methyl)pyridin-2-yl)-[1,1′-biphenyl]-3-yl)-9-methyl-2-(((((S)-5-oxopyrrolidin-2-yl)methyl)amino)methyl)-4H-pyrido[1,2-a]pyrimidin-4-one

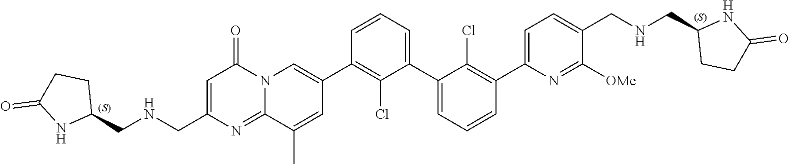

### 7-Bromo-2-(chloromethyl)-9-methyl-4H-pyrido[1,2-a]pyrimidin-4-one

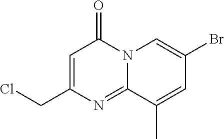

A mixture of 5-bromo-3-methylpyridin-2-amine (5 g, 26.7 mmol) and ethyl 4-chloro-3-oxobutanoate (6.16 g, 37.4 mmol) in the poly phosphoric acid (10 mL) was stirred at 110° C. for 0.5 h. The mixture was diluted with water (30 mL) and stirred at 0° C. for 1 h. The mixture was filtered and washed with water/EtOAc (2:1, 150 mL). The filtrate was extracted with EtOAc (3×100 mL). The combined organic phase was dried over anhydrous sulfate, filtered and concentrated under reduced pressure. The crude product was triturated with EtOAc (50 mL) at rt for 0.5 h to afford 7-bromo-2-(chloromethyl)-9-methyl-4H-pyrido[1,2-a]pyrimidin-4-one (4.3 g, crude) as a black solid. ¹H NMR (400 MHz, CDCl₃): δ 8.99 (d, J=1.6 Hz, 1H), 7.59 (d, J=1.2 Hz, 1H), 6.61 (s, 1H), 4.46 (s, 2H), 2.50 (s, 3H).

### (S)-7-Bromo-9-methyl-2-((((5-oxopyrrolidin-2-yl)methyl)amino)methyl)-4H-pyrido[1,2-a]pyrimidin-4-one

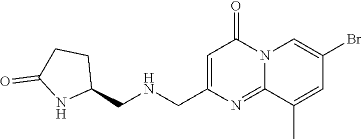 [SMILES: Cc1cc(Br)cn2c(=O)cc(CNC[C@@H]3CCC(=O)N3)nc12]

A mixture of 7-bromo-2-(chloromethyl)-9-methyl-4H-pyrido[1,2-a]pyrimidin-4-one (1.5 g, 5.22 mmol), (5S)-5-(aminomethyl)pyrrolidin-2-one, HCl (1.80 g, 11.9 mmol) and N,N-diisopropylethylamine (2.25 mL, 12.9 mmol) in DMF (50 mL) was stirred at rt for 24 h under N₂. Compound (S)-7-bromo-9-methyl-2-((((5-oxopyrrolidin-2-yl)methyl)amino)methyl)-4H-pyrido[1,2-a]pyrimidin-4-one (1.9 g, crude, m/z: 365 [M+H]⁺ observed) was obtained as a DMF solution. The crude product was used directly without further purification.

### (S)-tert-Butyl((7-bromo-9-methyl-4-oxo-4H-pyrido[1,2-a]pyrimidin-2-yl)methyl)((5-oxopyrrolidin-2-yl)methyl)carbamate

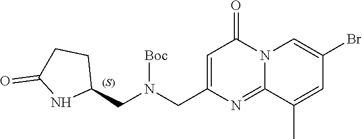

To a solution of(S)-7-bromo-9-methyl-2-((((5-oxopyrrolidin-2-yl)methyl)amino)methyl)-4H-pyrido[1,2-a]pyrimidin-4-one (1.91 g, 5.23 mmol) in DMF (50 mL) was added tert-butoxycarbonyl tert-butyl carbonate (3.42 g, 15.7 mmol) and the TEA (2.18 mL, 15.7 mmol). The mixture was stirred at rt for 12 h under N₂. The mixture was diluted with water (200 mL) and extracted with EtOAc (200 mL×2). The combined organic phase was dried over anhydrous sulfate, filtered and concentrated under reduced pressure. The residue was purified by normal phase SiO₂ chromatography (0-100% EtOAc/petroleum ether) to afford tert-butyl (S)-tert-butyl((7-bromo-9-methyl-4-oxo-4H-pyrido[1,2-a]pyrimidin-2-yl)methyl)((5-oxopyrrolidin-2-yl)methyl)carbamate (2 g, semi-purified product, m/z: 465 [M]⁺, 467 [M+2]⁺ observed).

### (S)-tert-Butyl((7-(2,2′-dichloro-3′-(5-formyl-6-methoxypyridin-2-yl)-[1,1′-biphenyl]-3-yl)-9-methyl-4-oxo-4H-pyrido[1,2-a]pyrimidin-2-yl)methyl)((5-oxopyrrolidin-2-yl)methyl)carbamate

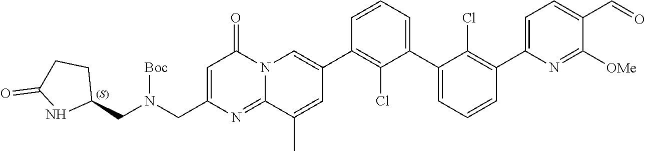

To a mixture of tert-butyl (S)-tert-butyl((7-bromo-9-methyl-4-oxo-4H-pyrido[1,2-a]pyrimidin-2-yl)methyl)((5-oxopyrrolidin-2-yl)methyl)carbamate (400 mg, 0.86 mmol) and 6-(2,2′-dichloro-3′-(4,4,5,5-tetramethyl-1,3,2-dioxaborolan-2-yl)-[1,1′-biphenyl]-3-yl)-2-methoxynicotinaldehyde (416 mg, 0.86 mmol) in dioxane/water (5:1, 12 mL) was added potassium carbonate (356 mg, 2.58 mmol), [1,1-bis(diphenylphosphino)ferrocene]palladium(II) chloride dichloromethane complex (70.2 mg, 0.08 mmol). The mixture was stirred at 80° C. for 1 h under N₂. The mixture was filtered and the filtrate was concentrated. The residue was purified by normal phase SiO₂ chromatography (0-100% EtOAc/petroleum ether) to afford (S)-tert-butyl((7-(2,2′-dichloro-3′-(5-formyl-6-methoxypyridin-2-yl)-[1,1′-biphenyl]-3-yl)-9-methyl-4-oxo-4H-pyrido[1,2-a]pyrimidin-2-yl)methyl)((5-oxopyrrolidin-2-yl)methyl)carbamate (400 mg, 62% yield) as a yellow solid. ¹H NMR (400 MHz, DMSO-d₆): δ 10.28 (s, 1H), 8.86 (s, 1H), 8.20 (d, J=8 Hz, 1H), 8.02 (s, 1H), 7.75-7.73 (m, 2H), 7.60 (t, J=7.2 Hz, 2H), 7.54 (d, J=1.6 Hz, 1H), 7.52 (d, J=1.6 Hz, 1H), 7.47 (d, J=7.6 Hz, 1H), 6.22 (d, J=7.6 Hz, 1H), 4.49-4.38 (m, 2H), 4.05 (s, 3H), 3.80-3.74 (m, 2H), 2.54 (s, 3H), 2.16-2.04 (m, 4H), 1.74-1.71 (m, 2H), 1.43-1.36 (m, 9H).

### tert-butyl ((7-(2,2′-dichloro-3′-(6-methoxy-5-(((((S)-5-oxopyrrolidin-2-yl)methyl)amino) methyl)pyridin-2-yl)-[1,1′-biphenyl]-3-yl)-9-methyl-4-oxo-4H-pyrido[1,2-a]pyrimidin-2-yl)methyl)(((S)-5-oxopyrrolidin-2-yl)methyl)carbamate

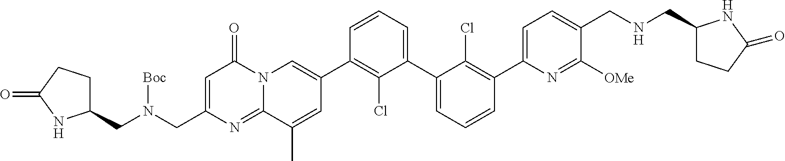 [SMILES: COc1nc(-c2cccc(-c3cccc(-c4cc(C)c5nc(CN(C[C@@H]6CCC(=O)N6)C(=O)OC(C)(C)C)cc(=O)n5c4)c3Cl)c2Cl)ccc1CNC[C@@H]1CCC(=O)N1]

To a mixture of(S)-tert-butyl((7-(2,2′-dichloro-3′-(5-formyl-6-methoxypyridin-2-yl)-[1,1′-biphenyl]-3-yl)-9-methyl-4-oxo-4H-pyrido[1,2-a]pyrimidin-2-yl)methyl)((5-oxopyrrolidin-2-yl)methyl)carbamate (200 mg, 269 umol) and (5S)-5-(aminomethyl)pyrrolidin-2-one, HCl (122 mg, 808 umol) in DCM (10 mL) was added sodium acetate (66.3 mg, 808 umol). The mixture was stirred at rt for 0.5 h under N₂. sodium triacetoxyborohydride (171 mg, 808 umol) was added, and the mixture was stirred at rt for 0.5 h under N₂. Compound tert-butyl ((7-(2,2′-dichloro-3′-(6-methoxy-5-(((((S)-5-oxopyrrolidin-2-yl)methyl)amino)methyl)pyridin-2-yl)-[1,1′-biphenyl]-3-yl)-9-methyl-4-oxo-4H-pyrido[1,2-a]pyrimidin-2-yl)methyl)(((S)-5-oxopyrrolidin-2-yl)methyl)carbamate (crude, m/z: 840 [M+H]⁺ observed) was obtained as a DCM solution (10 mL). The crude product was used directly without further purification.

### 7-(2,2′-Dichloro-3′-(6-methoxy-5-(((((S)-5-oxopyrrolidin-2-yl)methyl)amino)methyl)pyridin-2-yl)-[1,1′-biphenyl]-3-yl)-9-methyl-2-(((((S)-5-oxopyrrolidin-2-yl)methyl)amino)methyl)-4H-pyrido[1,2-a]pyrimidin-4-one

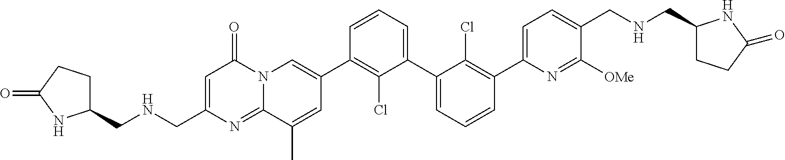 [SMILES: COc1nc(-c2cccc(-c3cccc(-c4cc(C)c5nc(CNC[C@@H]6CCC(=O)N6)cc(=O)n5c4)c3Cl)c2Cl)ccc1CNC[C@@H]1CCC(=O)N1]

To a solution of tert-butyl ((7-(2,2′-dichloro-3′-(6-methoxy-5-(((((S)-5-oxopyrrolidin-2-yl)methyl)amino)methyl)pyridin-2-yl)-[1,1′-biphenyl]-3-yl)-9-methyl-4-oxo-4H-pyrido[1,2-a]pyrimidin-2-yl)methyl)(((S)-5-oxopyrrolidin-2-yl)methyl)carbamate in DCM (10 mL) was added trifluoroacetic acid (5.50 mL, 74.3 mmol). The mixture was stirred at rt for 0.5 h. The mixture was concentrated and the residue was purified by reverse phase HPLC to afford 7-(2,2′-dichloro-3′-(6-methoxy-5-(((((S)-5-oxopyrrolidin-2-yl)methyl)amino)methyl)pyridin-2-yl)-[1,1′-biphenyl]-3-yl)-9-methyl-2-(((((S)-5-oxopyrrolidin-2-yl)methyl)amino)methyl)-4H-pyrido[1,2-a]pyrimidin-4-one (27.1 mg, m/z: 740 [M+H]⁺ observed). ¹H NMR (400 MHz, DMSO-d₆): δ 8.87 (d, J=1.6 Hz, 1H), 8.04 (s, 1H), 7.90 (d, J=7.6 Hz, 1H), 7.68-7.49 (m, 8H), 7.35 (d, J=7.2 Hz, 1H), 6.57 (s, 1H), 4.03-4.00 (m, 4H), 3.95 (s, 3H), 3.80-3.74 (m, 2H), 2.91-2.90 (m, 2H), 2.80-2.79 (m, 2H), 2.48 (s, 3H), 2.18-2.09 (m, 6H), 1.75-1.71 (m, 2H).

### Example 289: Methyl 1-((6-(2,2′-dichloro-3′-(3-((3-(methoxycarbonyl)azetidin-1-yl)methyl)-1,6-naphthyridin-7-yl)-[1,1′-biphenyl]-3-yl)-2-methoxypyridin-3-yl)methyl)azetidine-3-carboxylate

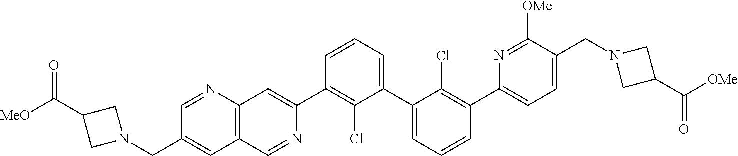 [SMILES: COC(=O)C1CN(Cc2cnc3cc(-c4cccc(-c5cccc(-c6ccc(CN7CC(C(=O)OC)C7)c(OC)n6)c5Cl)c4Cl)ncc3c2)C1]

To a mixture of 7-(2,2′-dichloro-3′-(5-formyl-6-methoxypyridin-2-yl)-[1,1′-biphenyl]-3-yl)-1,6-naphthyridine-3-carbaldehyde (100 mg, 0.19 mmol) and methyl azetidine-3-carboxylate, HCl (177 mg, 1.17 mmol) in DCM (10 mL) was added sodium acetate (95.7 mg, 1.17 mmol). The mixture was stirred at rt for 0.5 h under N₂. Sodium triacetoxyborohydride (247 mg, 1.17 mmol) was added and the mixture was stirred at rt for 2 h under N₂. The mixture was filtered, and the filtrate was concentrated. The residue was purified by reverse phase HPLC to afford methyl 1-((6-(2,2′-dichloro-3′-(3-((3-(methoxycarbonyl)azetidin-1-yl)methyl)-1,6-naphthyridin-7-yl)-[1,1′-biphenyl]-3-yl)-2-methoxypyridin-3-yl)methyl)azetidine-3-carboxylate (14.7 mg, 19.32 umol, 9.94% yield), m/z: 712 [M+H]⁺ observed. ¹H NMR (400 MHz, DMSO-d₆): δ 9.58 (s, 1H), 9.13 (d, J=1.6 Hz, 1H), 8.54 (d, J=1.2 Hz, 1H), 8.19 (s, 1H), 7.82-7.81 (m, 1H), 7.80-7.72 (m, 2H), 7.66-7.54 (m, 4H), 7.33 (d, J=7.6 Hz, 1H), 3.97 (s, 3H), 3.90 (s, 2H), 3.70 (s, 6H), 3.62-3.59 (m, 2H), 2.56-2.51 (m, 4H).

### Example 304: (S)-Methyl 1-((7-(2,2′-dichloro-3′-(6-methoxy-5-((((5-oxopyrrolidin-2-yl)methyl)amino) methyl)pyridin-2-yl)-[1,1′-biphenyl]-3-yl)-1,6-naphthyridin-3-yl)methyl)azetidine-3-carboxylate

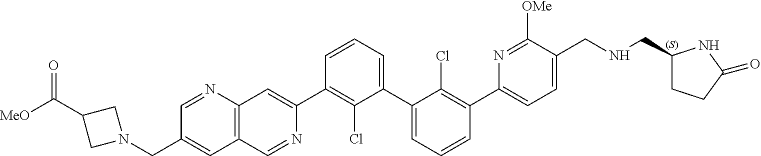

### (S)-Methyl 1-((7-(3′-(5-(((tert-butoxycarbonyl)((5-oxopyrrolidin-2-yl)methyl)amino) methyl)-6-methoxypyridin-2-yl)-2,2′-dichloro-[1,1′-biphenyl]-3-yl)-1,6-naphthyridin-3-yl)methyl)azetidine-3-carboxylate

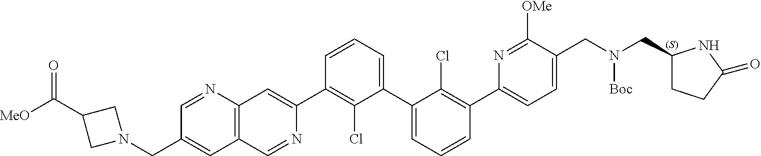

To a mixture of (S)-tert-butyl ((6-(2,2′-dichloro-3′-(3-formyl-1,6-naphthyridin-7-yl)-[1,1′-biphenyl]-3-yl)-2-methoxypyridin-3-yl)methyl)((5-oxopyrrolidin-2-yl)methyl)carbamate (200 mg, 0.28 mmol) in DCM (8 mL) was added methyl azetidine-3-carboxylate, HCl (63.8 mg, 0.42 mmol) and sodium acetate (69.1 mg, 0.84 mmol). After 3 h, sodium triacetoxyborohydride (178 mg, 0.84 mmol) was added and the stirring continued for 2 h at rt. The solvent was evaporated to afford methyl 1-[[7-[3-[3-[5-[[tert-butoxycarbonyl-[[(2S)-5-oxopyrrolidin-2-yl]methyl]amino]methyl]-6-methoxy-2-pyridyl]-2-chloro-phenyl]-2-chloro-phenyl]-1,6-naphthyridin-3-yl]methyl]azetidine-3-carboxylate. The crude product was used directly without any purification.

### (S)-Methyl 1-((7-(2,2′-dichloro-3′-(6-methoxy-5-((((5-oxopyrrolidin-2-yl)methyl)amino) methyl)pyridin-2-yl)-[1,1′-biphenyl]-3-yl)-1,6-naphthyridin-3-yl)methyl)azetidine-3-carboxylate

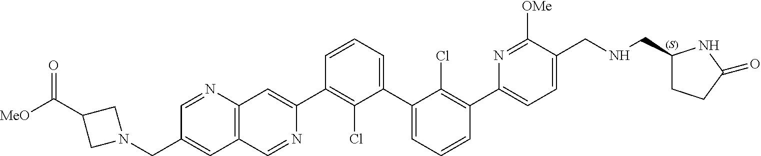

A mixture of(S)-methyl 1-((7-(3′-(5-(((tert-butoxycarbonyl)((5-oxopyrrolidin-2-yl) methyl)amino)methyl)-6-methoxypyridin-2-yl)-2,2′-dichloro-[1,1′-biphenyl]-3-yl)-1,6-naphthyridin-3-yl)methyl)azetidine-3-carboxylate (230 mg, 0.28 mmol) and TFA (1.84 mL) in DCM (8 mL) was stirred for 1 h at rt. The reaction was concentrated in vacuo and the residue was purified by reverse phase HPLC to afford (S)-methyl 1-((7-(2,2′-dichloro-3′-(6-methoxy-5-((((5-oxopyrrolidin-2-yl)methyl)amino)methyl)pyridin-2-yl)-[1,1′-biphenyl]-3-yl)-1,6-naphthyridin-3-yl)methyl)azetidine-3-carboxylate as a light-yellow solid (63.2 mg, 28% yield). m/z: 711 [M+H]⁺. ¹H NMR (400 MHz, DMSO-d₆): δ 9.58 (s, 1H), 9.18 (s, 1H), 8.91-8.87 (m, 1H), 8.74-8.63 (m, 2H), 8.20 (s, 1H), 7.97 (d, J=7.6 Hz, 1H), 7.78 (dd, J=7.6 Hz, J=1.6 Hz, 1H), 7.71-7.69 (m, 1H), 7.66-7.53 (m, 5H), 7.43 (d, J=7.6 Hz, 1H), 4.67-4.58 (m, 3H), 4.24 (s, 4H), 4.01 (s, 3H), 3.93-3.88 (m, 1H), 3.71 (s, 4H), 3.20-3.08 (m, 3H), 2.26-2.15 (m, 3H), 1.84-1.77 (m, 1H).

### Example 333: (S)-5-((((6-(2,2′-Dichloro-3′-(3-((((S)-2-hydroxypropyl)amino)methyl)-1,6-naphthyridin-7-yl)-[1,1′-biphenyl]-3-yl)-2-methoxypyridin-3-yl)methyl)amino)methyl)pyrrolidin-2-one

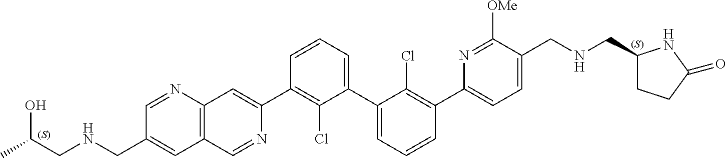

### (S)-tert-Butyl ((6-(2,2′-dichloro-3′-(3-formyl-1,6-naphthyridin-7-yl)-[1,1′-biphenyl]-3-yl)-2-methoxypyridin-3-yl)methyl)((5-oxopyrrolidin-2-yl)methyl)carbamate

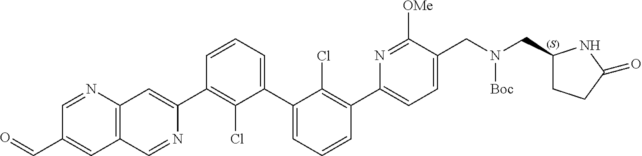

To a mixture of 7-chloro-1,6-naphthyridine-3-carbaldehyde (200 mg, 1.04 mmol), tert-butyl N-[[6-[2-chloro-3-[2-chloro-3-(4,4,5,5-tetramethyl-1,3,2-dioxaborolan-2-yl)phenyl]phenyl]-2-methoxy-3-pyridyl]methyl]-N-[[(2S)-5-oxopyrrolidin-2-yl]methyl]carbamate (1.06 g, 1.56 mmol) and triphenylphosphine (54.47 mg, 0.21 mmol) in EtOH/toluene/water (1/4/1 (v/v/v), 24 mL) was added tetrakis(triphenylphosphine) palladium(0) (60.0 mg, 0.052 mmol), followed by sodium carbonate (220 mg, 2.08 mmol). After stirring the reaction at 110° C. for 2h, the reaction was diluted with water (50 mL) and extracted with EtOAc (100 mL). The organic layer was dried over anhydrous sodium sulfate, filtered and concentrated in vacuo. The residue was purified by normal phase SiO₂ chromatography (0-100% EtOAc/petroleum ether in 30% THF) to afford (S)-tert-butyl ((6-(2,2′-dichloro-3′-(3-formyl-1,6-naphthyridin-7-yl)-[1,1′-biphenyl]-3-yl)-2-methoxypyridin-3-yl)methyl)((5-oxopyrrolidin-2-yl)methyl)carbamate as a yellow solid (670 mg, 56% yield, 62.8% purity). ¹H NMR (400 MHz, CDCl₃): δ 10.24 (s, 1H), 9.51 (d, J=2.0 Hz, 1H), 8.76 (s, 1H), 8.35 (s, 1H), 7.60-7.19 (m, 9H), 4.71 (d, J=6.0 Hz, 1H), 4.42 (s, 2H), 3.96 (s, 3H), 3.82 (t, J=6.0 Hz, 1H), 3.30-3.29 (m, 2H), 2.20-2.11 (m, 3H), 1.71-1.70 (m, 1H), 1.53 (s, 9H).

### tert-Butyl ((6-(2,2′-dichloro-3′-(3-((((S)-2-hydroxypropyl)amino)methyl)-1,6-naphthyridin-7-yl)-[1,1′-biphenyl]-3-yl)-2-methoxypyridin-3-yl)methyl)(((S)-5-oxopyrrolidin-2-yl)methyl)carbamate

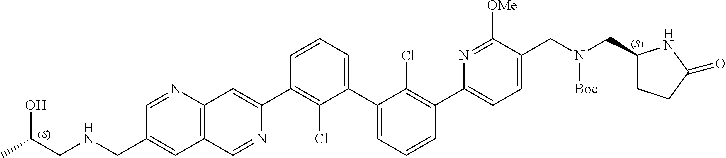

To a mixture of (S)-tert-butyl ((6-(2,2′-dichloro-3′-(3-formyl-1,6-naphthyridin-7-yl)-[1,1′-biphenyl]-3-yl)-2-methoxypyridin-3-yl)methyl)((5-oxopyrrolidin-2-yl)methyl)carbamate (170 mg, 0.24 mmol) in MeOH (4 mL) was added (S)-1-aminopropan-2-ol (56.4 uL, 0.72 mmol) and sodium acetate (97.8 mg, 1.19 mmol). After 1 h, sodium cyanoborohydride (22.5 mg, 0.36 mmol) was added and the mixture was stirred for 11 h at rt. Sodium cyanoborohydride (22 mg) was added and the reaction was stirred for another 16 h. The reaction mixture was concentrated in vacuo. The residue was purified by normal phase SiO₂ chromatography (0-100% EtOAc/petroleum ether to 0-40% MeOH/EtOAc) to afford tert-butyl ((6-(2,2′-dichloro-3′-(3-((((S)-2-hydroxypropyl)amino)methyl)-1,6-naphthyridin-7-yl)-[1,1′-biphenyl]-3-yl)-2-methoxypyridin-3-yl)methyl)(((S)-5-oxopyrrolidin-2-yl)methyl)carbamate as a yellow solid (200 mg, 78% yield). m/z: 771 [M+H]⁺.

### (S)-5-((((6-(2,2′-Dichloro-3′-(3-((((S)-2-hydroxypropyl)amino)methyl)-1,6-naphthyridin-7-yl)-[1,1′-biphenyl]-3-yl)-2-methoxypyridin-3-yl)methyl)amino)methyl)pyrrolidin-2-one

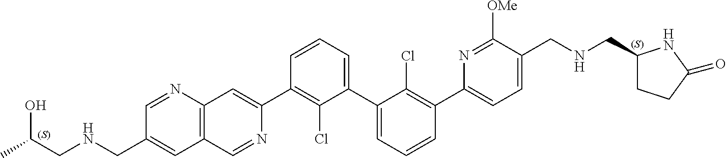

To a solution of tert-butyl ((6-(2,2′-dichloro-3′-(3-((((S)-2-hydroxypropyl)amino)methyl) −1,6-naphthyridin-7-yl)-[1,1′-biphenyl]-3-yl)-2-methoxypyridin-3-yl)methyl)(((S)-5-oxopyrrolidin-2-yl)methyl)carbamate (190 mg, 0.25 mmol) in DCM (7 mL), TFA (1.5 mL, 20.3 mmol) was added. The reaction was stirred for 0.5 h at rt. The reaction was concentrated, and the residue was purified by reverse phase HPLC to afford (S)-5-((((6-(2,2′-dichloro-3′-(3-((((S)-2-hydroxypropyl)amino)methyl)-1,6-naphthyridin-7-yl)-[1,1′-biphenyl]-3-yl)-2-methoxypyridin-3-yl)methyl)amino)methyl)pyrrolidin-2-one as a yellow solid (26.6 mg). m/z: 671 [M+H]⁺. ¹H NMR (400 MHz, DMSO-d₆): δ 9.51 (s, 1H), 9.16 (d, J=2.0 Hz, 1 H), 8.53 (s, 1H), 8.17 (d, J=12.8 Hz, 1H), 7.84 (d, J=7.6 Hz, 1H), 7.77 (dd, J=1.6 Hz, J=7.6 Hz, 1H), 7.70-7.68 (m, 2H), 7.61-7.48 (m, 4H), 7.29 (d, J=7.6 Hz, 1H), 4.02 (s, 2 H), 3.93 (s, 3H), 3.77-3.62 (m, 6H), 2.57 (d, J=6.0 Hz, 2H), 2.15-2.08 (m, 4H), 1.72-1.69 (m, 1H).

### Example 334: (S)-5-((((6-(2,2′-Dichloro-3′-(3-(((2-hydroxyethyl)amino)methyl)-1,6-naphthyridin-7-yl)-[1,1′-biphenyl]-3-yl)-2-methoxypyridin-3-yl)methyl)amino)methyl)pyrrolidin-2-one

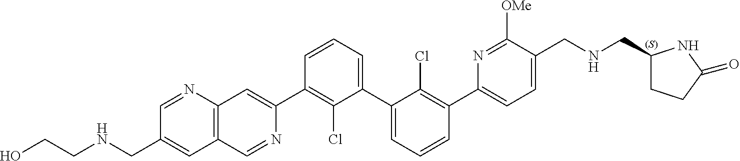

### (S)-tert-Butyl ((6-(2,2′-dichloro-3′-(3-(((2-hydroxyethyl)amino)methyl)-1,6-naphthyridin-7-yl)-[1,1′-biphenyl]-3-yl)-2-methoxypyridin-3-yl)methyl)((5-oxopyrrolidin-2-yl)methyl)carbamate

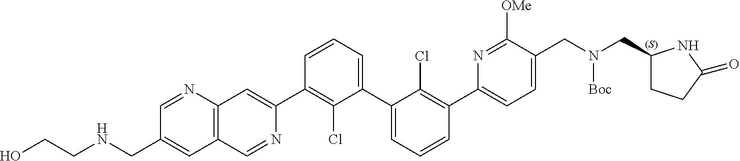

To a solution of (S)-tert-butyl ((6-(2,2′-dichloro-3′-(3-formyl-1,6-naphthyridin-7-yl)-[1,1′-biphenyl]-3-yl)-2-methoxypyridin-3-yl)methyl)((5-oxopyrrolidin-2-yl)methyl)carbamate (200 mg, 0.28 mmol) and 2-aminoethanol (51.4 mg, 0.84 mmol) in DCM/MeOH (1:1 6 mL), sodium acetate (115 mg, 1.40 mmol) was added. The mixture was stirred at rt for 11 h. Sodium cyanoborohydride (106 mg, 1.68 mmol) was added to the mixture and stirring continued for 7 h. The reaction mixture was concentrated and the residue was purified by normal phase SiO₂ prep-TLC (0-50% EtOAc/MeOH) to afford (S)-tert-butyl ((6-(2,2′-dichloro-3′-(3-(((2-hydroxyethyl)amino)methyl)-1,6-naphthyridin-7-yl)-[1,1′-biphenyl]-3-yl)-2-methoxypyridin-3-yl)methyl)((5-oxopyrrolidin-2-yl)methyl)carbamate (100 mg, 47% yield) m/z: 757 [M+H]⁺ observed.

### (S)-5-((((6-(2,2′-Dichloro-3′-(3-(((2-hydroxyethyl)amino)methyl)-1,6-naphthyridin-7-yl)-[1,1′-biphenyl]-3-yl)-2-methoxypyridin-3-yl)methyl)amino)methyl)pyrrolidin-2-one

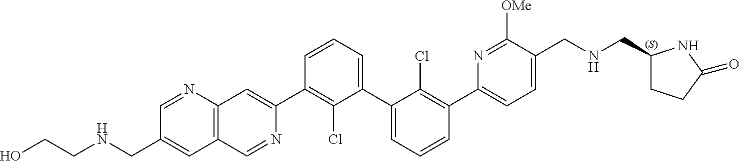

To a solution of (S)-tert-butyl ((6-(2,2′-dichloro-3′-(3-(((2-hydroxyethyl)amino)methyl)-1,6-naphthyridin-7-yl)-[1,1′-biphenyl]-3-yl)-2-methoxypyridin-3-yl)methyl)((5-oxopyrrolidin-2-yl)methyl)carbamate (140 mg, 0.18 mmol, 1 eq) in DCM (0.5 mL) was added trifluoroacetic acid (0.47 mL, 6.30 mmol). The mixture was stirred at rt for 2 h. The reaction mixture was concentrated and the residue was purified by reverse phase HPLC to afford (5S)-5-[[[6-[2-chloro-3-[2-chloro-3-[3-[(2-hydroxyethylamino)methyl]-1,6-naphthyridin-7-yl]phenyl]phenyl]-2-methoxy-3-pyridyl]methylamino]methyl]pyrrolidin-2-one (31.8 mg, 26% yield), m/z: 657 [M+H]⁺ observed. ¹H NMR (400 MHz, Methanol-d₄): δ 9.52 (s, 1H), 9.26 (d, J=2.0 Hz, 1H), 8.77 (s, 1H), 8.27 (s, 1H), 7.87 (d, J=7.6 Hz, 1H), 7.75 (dd, J=7.6 Hz, J=1.2 Hz, 1H), 7.68 (dd, J=7.2 Hz, J=1.6 Hz, 1H), 7.62 (t, J=7.6 Hz, 1H), 7.55-7.47 (m, 3H), 7.37 (d, J=7.2 Hz, 1H), 4.56 (s, 2H), 4.24 (s, 2H), 4.11 (s, 3H), 4.07-3.98 (m, 1H), 3.88 (t, J=5.2 Hz, 2H), 3.27 (t, J=4.8 Hz, 2H), 3.15 (m, 2H), 2.44-2.37 (m, 3H), 1.91-1.85 (m, 1H).

### Example 305: (S)-Methyl 2-(((7-(2,2′-dichloro-3′-(6-methoxy-5-((((5-oxopyrrolidin-2-yl)methyl)amino)methyl)pyridin-2-yl)-[1,1′-biphenyl]-3-yl)-1,6-naphthyridin-3-yl)methyl)amino)acetate

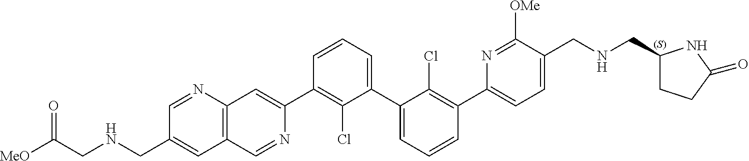

### (S)-Methyl 2-(((7-(3′-(5-(((tert-butoxycarbonyl)((5-oxopyrrolidin-2-yl)methyl)amino) methyl)-6-methoxypyridin-2-yl)-2,2′-dichloro-[1,1′-biphenyl]-3-yl)-1,6-naphthyridin-3-yl)methyl)amino)acetate

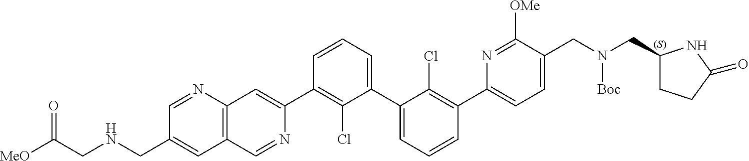

To a mixture of (S)-tert-butyl ((6-(2,2′-dichloro-3′-(3-formyl-1,6-naphthyridin-7-yl)-[1,1′-biphenyl]-3-yl)-2-methoxypyridin-3-yl)methyl)((5-oxopyrrolidin-2-yl)methyl)carbamate (150 mg, 0.21 mmol) in DCM/MeOH (1/1, 2 mL) was added methyl 2-aminoacetate, HCl (39.6 mg, 0.32 mmol) and sodium acetate (51.8 mg, 0.63 mmol). After stirring for 3 h, sodium cyanoborohydride (19.8 mg, 0.32 mmol) was added and stirring continued for 1 h at rt. The reaction was quenched with water (10 mL) and extracted with DCM (50 mL). The organic layer was dried over anhydrous sodium sulfate, filtered and the filtrate was concentrated in vacuo. The residue was purified by normal phase SiO₂ chromatography (0-100% EtOAc/petroleum ether to 0˜10% MeOH/Ethyl acetate) to afford (S)-methyl 2-(((7-(3′-(5-(((tert-butoxycarbonyl)((5-oxopyrrolidin-2-yl)methyl)amino)methyl)-6-methoxypyridin-2-yl)-2,2′-dichloro-[1,1′-biphenyl]-3-yl)-1,6-naphthyridin-3-yl)methyl)amino)acetate as a white solid (150 mg, crude).

### (S)-Methyl 2-(((7-(2,2′-dichloro-3′-(6-methoxy-5-((((5-oxopyrrolidin-2-yl)methyl)amino) methyl)pyridin-2-yl)-[1,1′-biphenyl]-3-yl)-1,6-naphthyridin-3-yl)methyl)amino)acetate

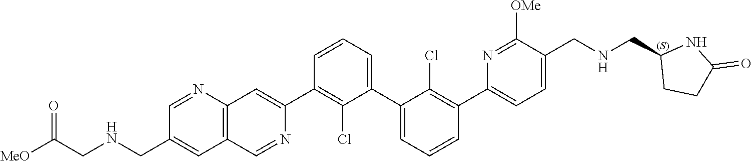

To a solution of methyl 2-[[7-[3-[3-[5-[[tert-butoxycarbonyl-[[(2S)-5-oxopyrrolidin-2-yl]methyl]amino]methyl]-6-methoxy-2-pyridyl]-2-chloro-phenyl]-2-chloro-phenyl]-1,6-naphthyridin-3-yl]methylamino]acetate (140 mg, 17.8 mmol) in DCM (10 mL) was added trifluoroacetic acid (2 mL) and stirred for 1 h at rt. The reaction mixture was concentrated in vacuo and the residue was purified by reverse phase HPLC to afford (S)-methyl 2-(((7-(2,2′-dichloro-3′-(6-methoxy-5-((((5-oxopyrrolidin-2-yl)methyl)amino)methyl)pyridin-2-yl)-[1,1′-biphenyl]-3-yl)-1,6-naphthyridin-3-yl)methyl)amino)acetate as a light-yellow solid (36.3 mg, 16.1% yield). m/z: 685 [M+H]⁺. ¹H NMR (400 MHz, DMSO-d₆): δ 9.51 (s, 1H), 9.14 (d, J=2.0 Hz, 1H), 8.51 (s, 1H), 8.17 (s, 1H), 8.14 (s, 1H), 7.84 (d, J=7.2 Hz, 1H), 7.77 (d, J=6.0 Hz, 1H), 7.76-7.68 (m, 2H), 7.63-7.48 (m, 4H), 7.29 (d, J=7.6 Hz, 1H), 4.01 (s, 2H), 3.93 (s, 3H), 3.73 (s, 2H), 3.64 (s, 4H), 3.43 (s, 1H), 2.57 (d, J=6.0 Hz, 4H), 2.15-2.09 (m, 3H), 1.73-1.69 (m, 1H).

### Example 341: (S)-2-(((8-(2,2′-Dichloro-3′-(6-methoxy-5-((((5-oxopyrrolidin-2-yl)methyl)amino)methyl) pyridin-2-yl)-[1,1′-biphenyl]-3-yl)-4-oxo-4H-pyrido[1,2-a]pyrimidin-2-yl)methyl)amino)-N,2-dimethylpropanamide

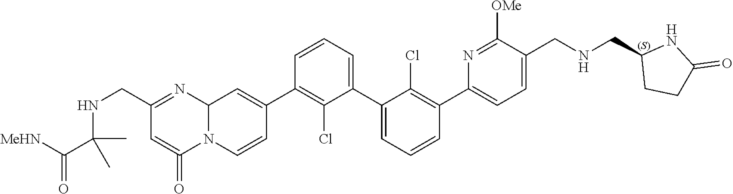

### 8-Bromo-2-(chloromethyl)-4H-pyrido[1,2-a]pyrimidin-4-one

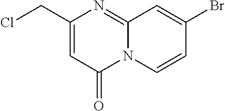 [SMILES: O=c1cc(CCl)nc2cc(Br)ccn12]

A mixture of 4-bromopyridin-2-amine (10 g, 57.8 mmol), ethyl 4-chloro-3-oxobutanoate (13.3 g, 80.9 mmol) in PPA (20 mL) was stirred at 110° C. for 0.5 h. The reaction mixture was diluted with water (1 L) and extracted with EtOAc (2×1 L). The combined organic layers were washed with brine (200 mL), dried over anhydrous sodium sulfate, filtered and concentrated under reduced pressure. The crude product was triturated with petroleum ether:ethyl acetate (10:1, 60 mL) at 25° C. for 0.5 h to afford 8-bromo-2-(chloromethyl)-4H-pyrido[1,2-a]pyrimidin-4-one was obtained as a brown solid (9 g, 56% yield. ¹H NMR (400 MHz, Methanol-d₄): δ 8.79 (d, J=7.6 Hz, 1H), 8.02 (d, J=2.0 Hz, 1H), 7.50 (d, J=2 Hz, J=7.6 Hz 1H), 6.58 (s, 1H), 4.66 (s, 2H).

### 8-Bromo-2-(hydroxymethyl)-4H-pyrido[1,2-a]pyrimidin-4-one

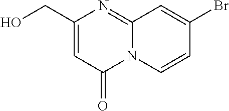 [SMILES: O=c1cc(CO)nc2cc(Br)ccn12]

To a solution of 8-bromo-2-(chloromethyl)-4H-pyrido[1,2-a]pyrimidin-4-one (5 g, 18.3 mmol) in water/DMSO (1:1, 100 mL) was added sodium bicarbonate (4.61 g, 54.8 mmol). The mixture was stirred at 85° C. for 24 h. The reaction was quenched with water (600 mL) and extracted with EtOAc (2×100 mL). The combined organic layers were washed with brine (100 mL), dried over anhydrous sodium sulfate, filtered and concentrated under reduced pressure to give 8-bromo-2-(hydroxymethyl)-4H-pyrido[1,2-a]pyrimidin-4-one as a white solid (3 g)¹H NMR (400 MHz, Methanol-d₄) δ=8.78 (d, J=7.6 Hz, 1H), 7.91 (d, J=1.6 Hz, 1H), 7.44 (d, J=2.4 Hz, J=7.6 Hz 1H), 6.47 (s, 1H), 5.60 (t, J=7 Hz, 1H), 4.45 (d, J=7 Hz, 2H)

### (S)-tert-Butyl ((6-(2,2′-dichloro-3′-(2-(hydroxymethyl)-4-oxo-4H-pyrido[1,2-a]pyrimidin-8-yl)-[1,1′-biphenyl]-3-yl)-2-methoxypyridin-3-yl)methyl)((5-oxopyrrolidin-2-yl)methyl) carbamate

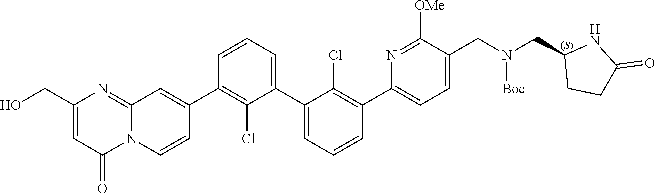

To a mixture of 8-bromo-2-(hydroxymethyl)-4H-pyrido[1,2-a]pyrimidin-4-one (0.5 g, 1.96 mmol) in dioxane/water (10:1, 8.8 mL) was added (S)-tert-butyl ((6-(2,2′-dichloro-3′-(4,4,5,5-tetramethyl-1,3,2-dioxaborolan-2-yl)-[1,1′-biphenyl]-3-yl)-2-methoxypyridin-3-yl)methyl)((5-oxopyrrolidin-2-yl)methyl)carbamate (1.47 g, 2.16 mmol), potassium phosphate (1.25 g, 5.88 mmol) and [1,1′-bis(di-tert-butylphosphino)ferrocene]dichloropalladium(II) (0.13 g, 0.20 mmol). The mixture was stirred at 110° C. for 0.5 h. The reaction mixture was diluted with water (30 mL) and extracted with EtOAc (3×30 ml). The combined organic layers were washed with brine (20 mL), dried over anhydrous sodium sulfate, filtered and concentrated under reduced pressure. The residue was purified by normal phase SiO₂ chromatography (0-100% EtOAc/petroleum ether) to afford (S)-tert-butyl ((6-(2,2′-dichloro-3′-(2-(hydroxymethyl)-4-oxo-4H-pyrido[1,2-a]pyrimidin-8-yl)-[1,1′-biphenyl]-3-yl)-2-methoxypyridin-3-yl)methyl)((5-oxopyrrolidin-2-yl)methyl)carbamate as a yellow solid (0.7 g, m/z:730.3.[M+H]⁺ observed).

### (S)-tert-Butyl ((6-(2,2′-dichloro-3′-(2-formyl-4-oxo-4H-pyrido[1,2-a]pyrimidin-8-yl)-[1,1′-biphenyl]-3-yl)-2-methoxypyridin-3-yl)methyl)((5-oxopyrrolidin-2-yl)methyl)carbamate

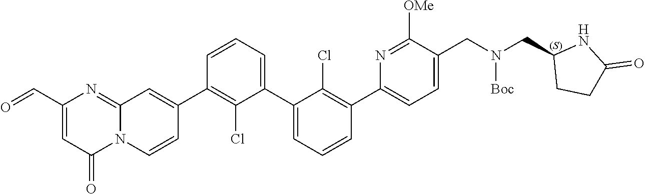

To a mixture of (S)-tert-butyl ((6-(2,2′-dichloro-3′-(2-(hydroxymethyl)-4-oxo-4H-pyrido[1,2-a]pyrimidin-8-yl)-[1,1′-biphenyl]-3-yl)-2-methoxypyridin-3-yl)methyl)((5-oxopyrrolidin-2-yl)methyl)carbamate (0.7 g, 0.96 mmol) in DCM (20 mL) was added Dess-Martin (0.81 g, 1.92 mmol). The reaction mixture was stirred at 25° C. for 2 h. The reaction mixture was filtered, and the filtrate was concentrated. The residue was purified by normal phase SiO₂ chromatography (0-100% EtOAc/petroleum ether) to afford (S)-tert-butyl ((6-(2,2′-dichloro-3′-(2-formyl-4-oxo-4H-pyrido[1,2-a]pyrimidin-8-yl)-[1,1′-biphenyl]-3-yl)-2-methoxypyridin-3-yl)methyl)((5-oxopyrrolidin-2-yl)methyl)carbamate as a yellow solid. (0.3 g, 42% yield, m/z: 728.1[M+H]⁺ observed).

### 2-Amino-N,2-dimethylpropanamide

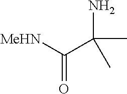

To an aqueous solution (40%) of methanamine (80 mL), methyl 2-amino-2-methylpropanoate, HCl (8 g, 52.1 mmol) was added. The reaction was stirred at rt for 12 h. The resulting solution was saturated with sodium chloride and extracted with dichloromethane (600 mL), dried over sodium sulfate, and filtered through magnesium sulfate. The filtrate was concentrated in vacuo to afford crude 2-amino-N,2-dimethylpropanamide (2.8 g, 46% yield) which was used in the next step without further purification.

### (S)-tert-Butyl ((6-(2,2′-dichloro-3′-(2-(((2-methyl-1-(methylamino)-1-oxopropan-2-yl)amino)methyl)-4-oxo-4H-pyrido[1,2-a]pyrimidin-8-yl)-[1,1′-biphenyl]-3-yl)-2-methoxypyridin-3-yl)methyl)((5-oxopyrrolidin-2-yl)methyl)carbamate

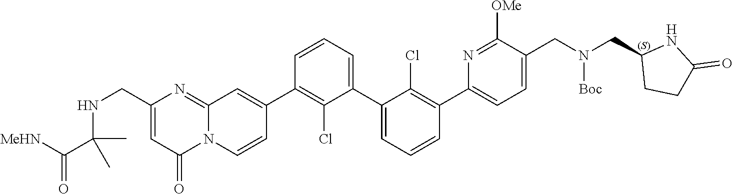

To a mixture of (S)-tert-butyl ((6-(2,2′-dichloro-3′-(2-formyl-4-oxo-4H-pyrido[1,2-a]pyrimidin-8-yl)-[1,1′-biphenyl]-3-yl)-2-methoxypyridin-3-yl)methyl)((5-oxopyrrolidin-2-yl)methyl) carbamate (0.15 g, 0.21 mmol) in DCM (5 mL) was added 2-amino-N,2-dimethylpropanamide (0.05 g, 0.41 mmol) and sodium acetate (0.05 g, 0.62 mmol). The mixture was stirred at 25° C. for 11 h. Sodium cyanoborohydride (0.03 g, 0.41 mmol) was added and the mixture was stirred at 25° C. for 1 h. The reaction mixture was concentrated under reduced pressure to give a (S)-tert-butyl ((6-(2,2′-dichloro-3′-(2-(((2-methyl-1-(methylamino)-1-oxopropan-2-yl)amino)methyl)-4-oxo-4H-pyrido[1,2-a]pyrimidin-8-yl)-[1,1′-biphenyl]-3-yl)-2-methoxypyridin-3-yl)methyl)((5-oxopyrrolidin-2-yl)methyl)carbamate as a yellow solid (0.2 g, crude, m/z: 828.3[M+H]⁺ observed).

### (S)-2-(((8-(2,2′-Dichloro-3′-(6-methoxy-5-((((5-oxopyrrolidin-2-yl)methyl)amino)methyl) pyridin-2-yl)-[1,1′-biphenyl]-3-yl)-4-oxo-4H-pyrido[1,2-a]pyrimidin-2-yl)methyl) amino)-N,2-dimethylpropanamide

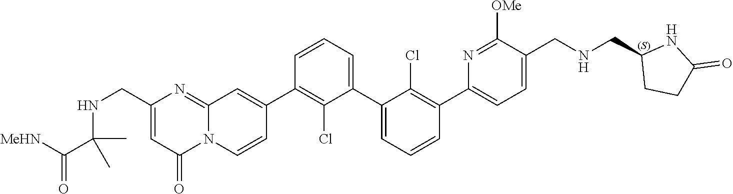

To a mixture of (S)-tert-butyl ((6-(2,2′-dichloro-3′-(2-(((2-methyl-1-(methylamino)-1-oxopropan-2-yl)amino)methyl)-4-oxo-4H-pyrido[1,2-a]pyrimidin-8-yl)-[1,1′-biphenyl]-3-yl)-2-methoxypyridin-3-yl)methyl)((5-oxopyrrolidin-2-yl)methyl)carbamate (0.2 g, 0.24 mmol) in DCM (3 mL) was added trifluoroacetic acid (0.2 mL). The mixture was stirred at 25° C. for 10 min. The solvent was evaporated under reduced pressure and the residue was purified by reverse phase HPLC to afford (S)-2-(((8-(2,2′-dichloro-3′-(6-methoxy-5-((((5-oxopyrrolidin-2-yl)methyl)amino)methyl)pyridin-2-yl)-[1,1′-biphenyl]-3-yl)-4-oxo-4H-pyrido[1,2-a]pyrimidin-2-yl)methyl)amino)-N,2-dimethylpropanamide as a yellow solid (37.3 mg, 19% yield, m/z:728.3[M+H]⁺ observed). ¹H NMR (400 MHz, Methanol-d₄): δ 9.17 (d, J=7.2 Hz, 1H), 7.97 (d, J=1.6 Hz, 1H), 7.92 (d, J=7.6 Hz, 1H), 7.69-7.57 (m, 6H), 7.46 (dd, J=2.0 Hz, J=7.6 Hz, 1H), 7.39 (d, J=7.6 Hz, 1H), 6.58 (s, 1H), 4.41-4.32 (m, 4H), 4.13-4.06 (m, 4H), 3.30-3.24 (m, 2H), 2.85 (s, 3H), 2.48-2.37 (m, 3H), 1.97-1.88 (m, 1H), 1.71 (s, 6H).

### Example 349: (S)-1-(((8-(2,2′-Dichloro-3′-(6-methoxy-5-((((5-oxopyrrolidin-2-yl)methyl)amino)methyl) pyridin-2-yl)-[1,1′-biphenyl]-3-yl)-4-oxo-4H-pyrido[1,2-a]pyrimidin-2-yl)methyl)amino)-N-methylcyclopropanecarboxamide

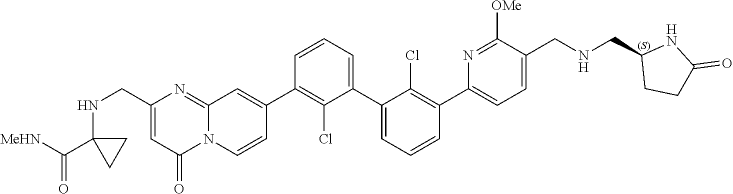

### Amino-N-methylcyclopropanecarboxamide

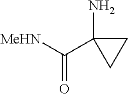

A mixture of methyl 1-aminocyclopropanecarboxylate (0.8 g, 6.95 mmol) in methylamine (5 mL, 40% in water) was stirred at rt for 5 h. The mixture was extracted with CH₂Cl₂ (5×20 mL). The combined organic phase was dried with anhydrous sodium sulfate, filtered and concentrated to afford the crude 1-amino-N-methylcyclopropanecarboxamide (0.26 g, 32% yield, m/z: 115 [M+H]⁺ observed) as a yellow oil which was used without further purification. ¹H NMR (400 MHz, CDCl₃): δ 7.61 (bs, 1H), 2.77 (d, J=5.2 Hz, 3H), 1.49 (s, 2H), 1.39-1.36 (m, 2H), 0.73-0.71 (m, 2H).

### (S)-tert-Butyl ((6-(2,2′-dichloro-3′-(2-(((1-(methylcarbamoyl)cyclopropyl)amino)methyl)-4-oxo-4H-pyrido[1,2-a]pyrimidin-8-yl)-[1,1′-biphenyl]-3-yl)-2-methoxypyridin-3-yl)methyl) ((5-oxopyrrolidin-2-yl)methyl)carbamate

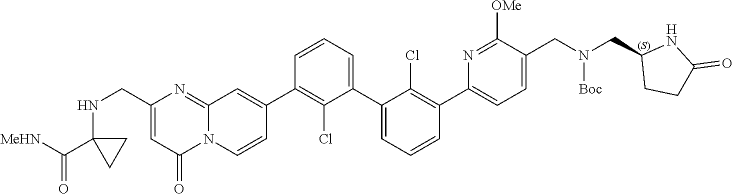

To a mixture of (S)-tert-butyl ((6-(2,2′-dichloro-3′-(2-formyl-4-oxo-4H-pyrido[1,2-a]pyrimidin-8-yl)-[1,1′-biphenyl]-3-yl)-2-methoxypyridin-3-yl)methyl)((5-oxopyrrolidin-2-yl)methyl)carbamate (0.18 g, 0.25 mmol) in CH₂Cl₂ (3 mL) was added sodium acetate (0.06 g, 0.74 mmol). The mixture was stirred at rt for 12 h. Sodium cyanoborohydride (0.05 g, 0.74 mmol) was added and the stirring continued for 0.5 h. The mixture was concentrated to afford the crude (S)-tert-butyl ((6-(2,2′-dichloro-3′-(2-(((1-(methylcarbamoyl)cyclopropyl)amino) methyl)-4-oxo-4H-pyrido[1,2-a]pyrimidin-8-yl)-[1,1′-biphenyl]-3-yl)-2-methoxypyridin-3-yl)methyl)((5-oxopyrrolidin-2-yl)methyl)carbamate (0.3 g, crude, m/z: 826 [M+H]⁺ observed) as a yellow solid which was used without further purification.

### (S)-1-(((8-(2,2′-Dichloro-3′-(6-methoxy-5-((((5-oxopyrrolidin-2-yl)methyl)amino)methyl) pyridin-2-yl)-[1,1′-biphenyl]-3-yl)-4-oxo-4H-pyrido[1,2-a]pyrimidin-2-yl)methyl)amino)-N-methylcyclopropanecarboxamide

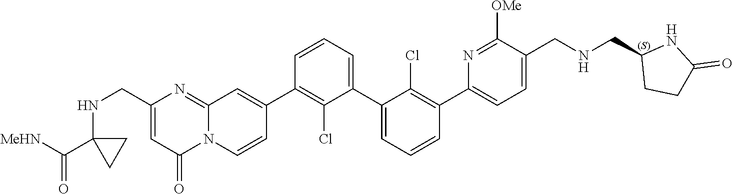

To a mixture of (S)-tert-butyl ((6-(2,2′-dichloro-3′-(2-(((1-(methylcarbamoyl)cyclopropyl) amino)methyl)-4-oxo-4H-pyrido[1,2-a]pyrimidin-8-yl)-[1,1′-biphenyl]-3-yl)-2-methoxypyridin-3-yl)methyl)((5-oxopyrrolidin-2-yl)methyl)carbamate (0.28 g, 0.34 mmol) in CH₂Cl₂ (1 mL) was added trifluoroacetic acid (2 mL). The mixture was stirred at rt for 1 h. The mixture was concentrated and the residue was purified by reverse phase HPLC to afford (S)-1-(((8-(2,2′-dichloro-3′-(6-methoxy-5-((((5-oxopyrrolidin-2-yl)methyl)amino)methyl)pyridin-2-yl)-[1,1′-biphenyl]-3-yl)-4-oxo-4H-pyrido[1,2-a]pyrimidin-2-yl)methyl)amino)-N-methylcyclopropane carboxamide (33.2 mg, 11% yield for two steps, m/z: 726 [M+H]⁺ observed). ¹H NMR (400 MHz, DMSO-d₆): δ 9.05 (d, J=8 Hz, 1H), 8.85-8.63 (m, 2H), 7.98-7.96 (m, 1H), 7.77-7.52 (m, 8H), 7.42 (d, J=7.6 Hz, 1H), 6.58 (s, 1H), 4.24 (m, 2H), 4.08-4.00 (m, 5H), 3.91 (m, 1H), 3.17-3.10 (m, 3H), 2.64 (d, J=4.4 Hz, 2H), 2.24-2.15 (m, 3H), 1.84-1.80 (m, 1H), 1.28 (m, 4H).

### Example 342: 8-(2,2′-Dichloro-3′-(6-methoxy-5-(((((S)-5-oxopyrrolidin-2-yl)methyl)amino)methyl)pyridin-2-yl)-[1,1′-biphenyl]-3-yl)-2-(((S)-4-hydroxy-2-oxopyrrolidin-1-yl)methyl)-4H-pyrido[1,2-a]pyrimidin-4-one

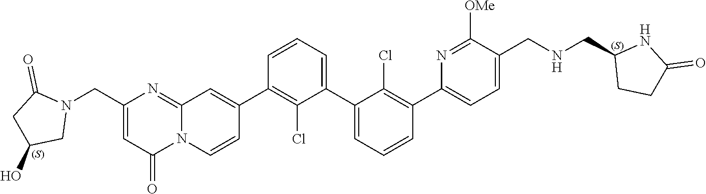

### (S)-8-Bromo-2-((4-hydroxy-2-oxopyrrolidin-1-yl)methyl)-4H-pyrido[1,2-a]pyrimidin-4-one

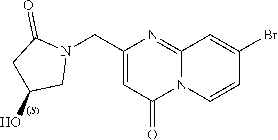

To a solution of 8-bromo-2-(chloromethyl)-4H-pyrido[1,2-a]pyrimidin-4-one (350 mg, 1.28 mmol) and and (S)-methyl 4-amino-3-hydroxybutanoate, HCl (300 mg, 1.77 mmol) in DMF (7 mL) was added potassium carbonate (531 mg, 3.84 mmol) and potassium iodide (63.7 mg, 0.38 mmol). The mixture was stirred at 80° C. for 2 h. The reaction mixture was quenched with water (50 mL) and extracted with EtOAc (2×30 mL). The aqueous layer was lyophilized under reduced pressure. The residue was diluted with MeOH (10 mL) and EtOAc (10 mL), filtered and concentrated to give (S)-8-bromo-2-((4-hydroxy-2-oxopyrrolidin-1-yl)methyl)-4H-pyrido[1,2-a]pyrimidin-4-one (1 g, crude, m/z: 338 [M]⁺, 340 [M+2]⁺ observed) as a yellow solid.

### tert-Butyl ((6-(2,2′-dichloro-3′-(2-(((S)-4-hydroxy-2-oxopyrrolidin-1-yl)methyl)-4-oxo-4H-pyrido[1,2-a]pyrimidin-8-yl)-[1,1′-biphenyl]-3-yl)-2-methoxypyridin-3-yl)methyl)(((S)-5-oxopyrrolidin-2-yl)methyl)carbamate

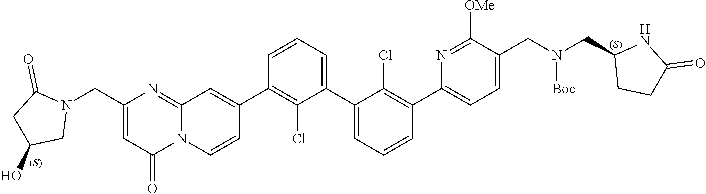

To a mixture of (S)-8-bromo-2-((4-hydroxy-2-oxopyrrolidin-1-yl)methyl)-4H-pyrido[1,2-a]pyrimidin-4-one (0.8 g, 828 umol, 35% purity) and (S)-tert-butyl ((6-(2,2′-dichloro-3′-(4,4,5,5-tetramethyl-1,3,2-dioxaborolan-2-yl)-[1,1′-biphenyl]-3-yl)-2-methoxypyridin-3-yl)methyl)((5-oxopyrrolidin-2-yl)methyl)carbamate (564 mg, 828 umol) in dioxane (8 mL) and water (2 mL) was added di-tert-butyl(cyclopentyl)phosphane;dichloropalladium;iron (54 mg, 82.8 umol) followed by potassium phosphate (526 mg, 2.48 mmol). The reaction was stirred for 0.5 h at 110° C. The mixture was diluted with water (10 mL) and extracted with EtOAc (30 mL). The organic layer was dried over anhydrous sodium sulfate, filtered and concentrated in vacuo. The residue was purified by normal phase SiO₂ chromatography (0-100% EtOAc/petroleum ether) to afford tert-butyl ((6-(2,2′-dichloro-3′-(2-(((S)-4-hydroxy-2-oxopyrrolidin-1-yl)methyl)-4-oxo-4H-pyrido[1,2-a]pyrimidin-8-yl)-[1,1′-biphenyl]-3-yl)-2-methoxypyridin-3-yl)methyl)(((S)-5-oxopyrrolidin-2-yl)methyl)carbamate (0.18 g, m/z: 813 [M+H]⁺ observed) as a yellow solid.

### 8-(2,2′-Dichloro-3′-(6-methoxy-5-(((((S)-5-oxopyrrolidin-2-yl)methyl)amino)methyl)pyridin-2-yl)-[1,1′-biphenyl]-3-yl)-2-(((S)-4-hydroxy-2-oxopyrrolidin-1-yl)methyl)-4H-pyrido[1,2-a]pyrimidin-4-one

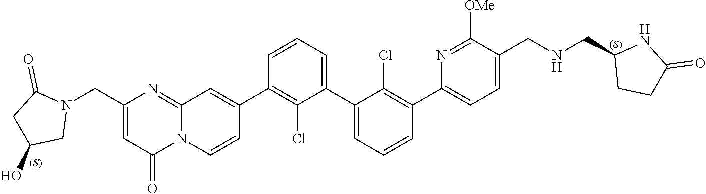

To a solution of tert-butyl ((6-(2,2′-dichloro-3′-(2-(((S)-4-hydroxy-2-oxopyrrolidin-1-yl)methyl)-4-oxo-4H-pyrido[1,2-a]pyrimidin-8-yl)-[1,1′-biphenyl]-3-yl)-2-methoxypyridin-3-yl)methyl)(((S)-5-oxopyrrolidin-2-yl)methyl)carbamate (170 mg, 0.21 mmol) in DCM (2 mL) was added trifluoroacetic acid (1 mL, 13.5 mmol). The mixture was stirred at rt for 12 h. The residue was purified by reverse phase HPLC to afford 8-(2,2′-dichloro-3′-(6-methoxy-5-(((((S)-5-oxopyrrolidin-2-yl)methyl)amino)methyl)pyridin-2-yl)-[1,1′-biphenyl]-3-yl)-2-(((S)-4-hydroxy-2-oxopyrrolidin-1-yl)methyl)-4H-pyrido[1,2-a]pyrimidin-4-one (76.9 mg, 46% yield, m/z: 713 [M+H]⁺ observed) as a white solid. ¹H NMR (400 MHz, Methanol-d₄): δ 9.09 (d, J=7.2 Hz, 1H), 7.81-7.79 (m, 2H), 7.69-7.59 (m, 3H), 7.55-7.51 (m, 3H), 7.42 (dd, J=8 Hz, J=2 Hz, 1H), 7.30 (d, J=7.6 Hz, 1H), 6.45 (s, 1H), 4.63-4.48 (m, 4H), 4.06 (s, 3H), 4.00 (d, J=2.4 Hz, 2H), 3.97-3.92 (m, 1H), 3.86-3.82 (m, 1H), 3.45-3.42 (m, 1H), 2.90-2.81 (m, 3H), 2.42-2.31 (m, 4H), 1.89-1.82 (m, 1H).

### Example 343: (S)-Methyl 1-((8-(2,2′-dichloro-3′-(6-methoxy-5-((((5-oxopyrrolidin-2-yl)methyl)amino) methyl)pyridin-2-yl)-[1,1′-biphenyl]-3-yl)-4-oxo-4H-pyrido[1,2-a]pyrimidin-2-yl)methyl) azetidine-3-carboxylate

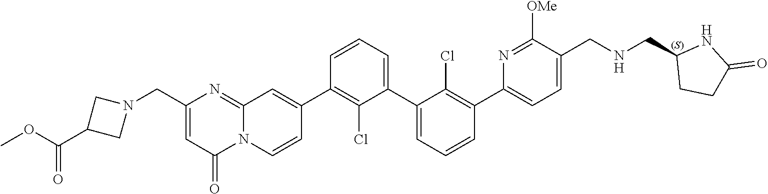

### (S)-Methyl 1-((8-(3′-(5-(((tert-butoxycarbonyl)((5-oxopyrrolidin-2-yl)methyl)amino)methyl)-6-methoxypyridin-2-yl)-2,2′-dichloro-[1,1′-biphenyl]-3-yl)-4-oxo-4H-pyrido[1,2-a]pyrimidin-2-yl)methyl)azetidine-3-carboxylate

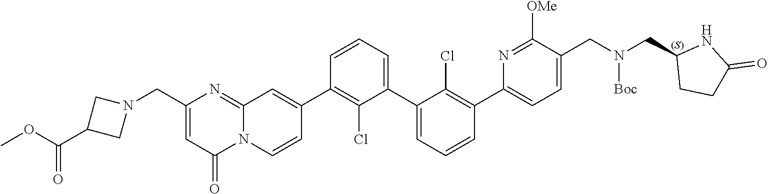

To a mixture of (S)-tert-butyl ((6-(2,2′-dichloro-3′-(2-formyl-4-oxo-4H-pyrido[1,2-a]pyrimidin-8-yl)-[1,1′-biphenyl]-3-yl)-2-methoxypyridin-3-yl)methyl)((5-oxopyrrolidin-2-yl)methyl)carbamate (0.13 g, 0.18 mmol) in DCM (5 mL) was added methyl azetidine-3-carboxylate, HCl (0.04 g, 0.27 mmol) and sodium acetate (0.04 g, 0.54 mmol). The mixture was stirred at 25° C. for 11 h. Sodium cyanoborohydride (0.03 g, 0.54 mmol) was added and the mixture was stirred at 25° C. for 1 h. The reaction mixture was concentrated under reduced pressure to give (S)-methyl 1-((8-(3′-(5-(((tert-butoxycarbonyl)((5-oxopyrrolidin-2-yl)methyl)amino)methyl)-6-methoxypyridin-2-yl)-2,2′-dichloro-[1,1′-biphenyl]-3-yl)-4-oxo-4H-pyrido[1,2-a]pyrimidin-2-yl)methyl)azetidine-3-carboxylate as a yellow solid (0.15 g, crude, m/z:827.1 [M+H]⁺ observed).

### (S)-Methyl 1-((8-(2,2′-dichloro-3′-(6-methoxy-5-((((5-oxopyrrolidin-2-yl)methyl)amino) methyl)pyridin-2-yl)-[1,1′-biphenyl]-3-yl)-4-oxo-4H-pyrido[1,2-a]pyrimidin-2-yl)methyl)azetidine-3-carboxylate

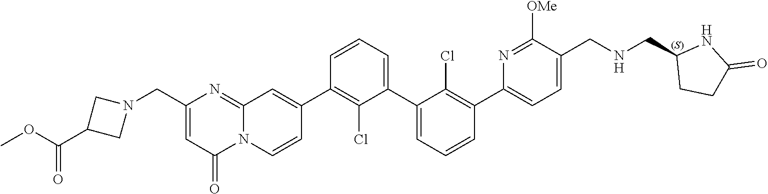

To a mixture of (S)-methyl 1-((8-(3′-(5-(((tert-butoxycarbonyl)((5-oxopyrrolidin-2-yl)methyl)amino)methyl)-6-methoxypyridin-2-yl)-2,2′-dichloro-[1,1′-biphenyl]-3-yl)-4-oxo-4H-pyrido[1,2-a]pyrimidin-2-yl)methyl)azetidine-3-carboxylate (0.15 g, 0.18 mmol) in DCM (3 mL) was added trifluoroacetic acid (1.50 mL). The mixture was stirred at 25° C. for 10 min. The reaction mixture was concentrated under reduced pressure and the residue was purified by reverse phase HPLC to afford (S)-methyl 1-((8-(2,2′-dichloro-3′-(6-methoxy-5-((((5-oxopyrrolidin-2-yl)methyl)amino)methyl)pyridin-2-yl)-[1,1′-biphenyl]-3-yl)-4-oxo-4H-pyrido[1,2-a]pyrimidin-2-yl)methyl)azetidine-3-carboxylate as a yellow solid (30.1 mg, 20% yield, m/z:727.2 [M+H]⁺ observed). ¹H NMR (400 MHz, Methanol-d₄) δ 9.10 (d, J=7.2 Hz, 1H), 7.84 (d, J=7.2 Hz, 1H), 7.81 (d, J=1.2 Hz, 1H), 7.69 (dd, J=1.6 Hz, J=7.6 Hz, 1H), 7.66-7.60 (m, 2H), 7.57-7.52 (m, 3H), 7.44 (dd, J=1.2 Hz, J=7.2 Hz, 1H), 7.34 (d, J=7.2 Hz, 1H), 6.51 (s, 1H), 4.19-4.09 (m, 5H), 4.02-3.95 (m, 5H), 3.86-3.76 (m, 2H), 3.66 (s, 3H), 3.61-3.50 (m, 1H), 3.10-3.00 (m, 2H), 2.48-2.34 (m, 3H), 1.95-1.86 (m, 1H).

### Example 404: 8-(2,2′-Dichloro-3′-(4-fluoro-6-methoxy-5-(((((S)-5-oxopyrrolidin-2-yl)methyl)amino) methyl)pyridin-2-yl)-[1,1′-biphenyl]-3-yl)-2-methyl-3-(((((S)-5-oxopyrrolidin-2-yl)methyl) amino)methyl)-4H-pyrido[1,2-a]pyrimidin-4-one

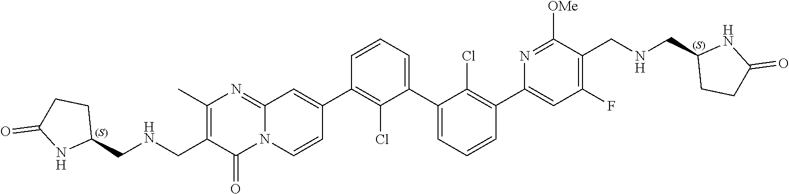

### 6-(3-Bromo-2-chlorophenyl)-4-fluoro-2-methoxynicotinaldehyde

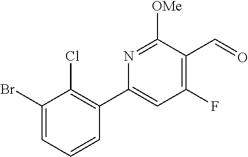 [SMILES: COc1nc(-c2cccc(Br)c2Cl)cc(F)c1C=O]

To a mixture of 6-chloro-4-fluoro-2-methoxynicotinaldehyde (2 g, 10.5 mmol), 2-(3-bromo-2-chlorophenyl)-4,4,5,5-tetramethyl-1,3,2-dioxaborolane (3.18 g, 10 mmol) in dioxane/water (6:1, 24 mL) was added potassium carbonate (4.37 g, 31.65 mmol), tetrakis(triphenylphosphine) platinum (1.22 g, 1.06 mmol). The mixture was stirred at 95° C. for 2 h under N₂. The mixture was filtered, and the filtrate was concentrated. The residue was purified by normal phase SiO₂ chromatography (0-10% EtOAc/petroleum ether) to afford 6-(3-bromo-2-chlorophenyl)-4-fluoro-2-methoxynicotinaldehyde (1.6 g, 44% yield) as a white solid. ¹H NMR (400 MHz, DMSO-d₆): δ 10.13 (s, 1H), 7.67 (dd, J₁=1.6 Hz, J₂=8 Hz, 1H), 7.45 (dd, J₁=1.6 Hz, J₂=7.6 Hz, 1H), 7.18 (d, J=6 Hz, 1H), 6.99 (d, J=10 Hz, 1H), 4.04 (s, 3H).

### 6-(2-Chloro-3-(4,4,5,5-tetramethyl-1,3,2-dioxaborolan-2-yl)phenyl)-4-fluoro-2-methoxynicotinaldehyde

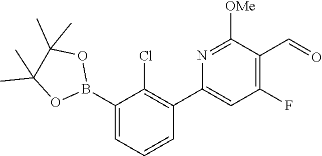 [SMILES: COc1nc(-c2cccc(B3OC(C)(C)C(C)(C)O3)c2Cl)cc(F)c1C=O]

To a mixture of 6-(3-bromo-2-chlorophenyl)-4-fluoro-2-methoxynicotinaldehyde (450 mg, 1.25 mmol), 4,4,4′,4′,5,5,5′,5′-octamethyl-2,2′-bi(1,3,2-dioxaborolane) (1.58 g, 6.23 mmol) in dioxane (10 mL) was added potassium acetate (245 mg, 2.49 mmol), [1,1′-bis(di-tert-butylphosphino) ferrocene]dichloropalladium(II) (91.2 mg, 0.12 mmol). The mixture was stirred at 95° C. for 2 h under N₂. The mixture was filtered and the filtrate was concentrated to give the residue. The residue was purified by normal phase SiO₂ chromatography (0-10% EtOAc/petroleum ether) to afford 6-(2-chloro-3-(4,4,5,5-tetramethyl-1,3,2-dioxaborolan-2-yl)phenyl)-4-fluoro-2-methoxynicotinaldehyde (700 mg) as a white solid. ¹H NMR (400 MHz, DMSO-d₆): δ 10.35 (s, 1H), 7.73-7.71 (m, 2H), 7.49 (t, J=7.6 Hz, 2H), 4.04 (s, 3H), 1.33 (s, 12H).

### 8-(2,2′-Dichloro-3′-(4-fluoro-5-formyl-6-methoxypyridin-2-yl)-[1,1′-biphenyl]-3-yl)-2-methyl-4-oxo-4H-pyrido[1,2-a]pyrimidine-3-carbaldehyde

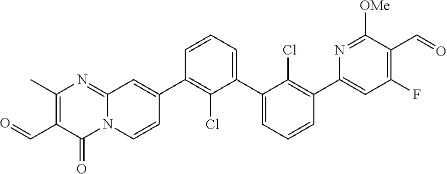 [SMILES: COc1nc(-c2cccc(-c3cccc(-c4ccn5c(=O)c(C=O)c(C)nc5c4)c3Cl)c2Cl)cc(F)c1C=O]

To a mixture of 6-(2-chloro-3-(4,4,5,5-tetramethyl-1,3,2-dioxaborolan-2-yl)phenyl)-4-fluoro-2-methoxynic (456.32 mg, 1.17 mmol), 8-(3-bromo-2-chlorophenyl)-2-methyl-4-oxo-4H-pyrido[1,2-a]pyrimidine-3-carbaldehyde (200 mg, 0.53 mmol) in THF/water (4:1, 10 mL) was added potassium phosphate (225 mg, 1.06 mmol), [1,1-bis(di-tert-butylphosphino)ferrocene] palladium(II) dichloride (34.5 mg, 0.05 mmol). The mixture was stirred at 80° C. for 2 h under N₂. The mixture was filtered, and the filtrate was concentrated to give the residue. The residue was purified by normal phase SiO₂ chromatography (0-70% EtOAc/petroleum ether) to afford 8-(2,2′-dichloro-3′-(4-fluoro-5-formyl-6-methoxypyridin-2-yl)-[1,1′-biphenyl]-3-yl)-2-methyl-4-oxo-4H-pyrido[1,2-a]pyrimidine-3-carbaldehyde (180 mg, 60% yield) as a yellow solid. ¹H NMR (400 MHz, DMSO-d₆): δ 10.46 (s, 1H), 10.31 (s, 1H), 9.23 (d, J=7.6 Hz, 1H), 7.95 (d, J=1.6 Hz, 1H), 7.84 (dd, J₁=1.6 Hz, J₂=7.6 Hz, 1H), 7.78-7.63 (m, 6H), 7.49 (d, J=10.4 Hz, 1H), 4.12 (s, 3H), 2.76 (s, 3H).

### 8-(2,2′-Dichloro-3′-(4-fluoro-6-methoxy-5-(((((S)-5-oxopyrrolidin-2-yl)methyl)amino) methyl)pyridin-2-yl)-[1,1′-biphenyl]-3-yl)-2-methyl-3-(((((S)-5-oxopyrrolidin-2-yl)methyl) amino)methyl)-4H-pyrido[1,2-a]pyrimidin-4-one

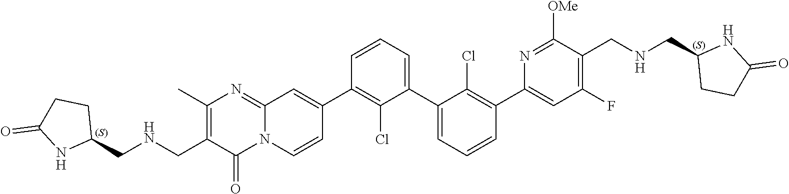

T a mixture of 8-(2,2′-dichloro-3′-(4-fluoro-5-formyl-6-methoxypyridin-2-yl)-[1,1′-biphenyl]-3-yl)-2-methyl-4-oxo-4H-pyrido[1,2-a]pyrimidine-3-carbaldehyde (170 mg, 0.30 mmol), (S)-5-(aminomethyl)pyrrolidin-2-one, HCl (182 mg, 1.21 mmol) in DCM (10 mL) was added sodium acetate (99.2 mg, 1.21 mmol) and sodium triacetoxyborohydride (256 mg, 1.21 mmol). The mixture was stirred at rt for 2 h under N₂. The reaction mixture was filtered and concentrated under reduced pressure to give a residue. The residue was purified by reverse phase HPLC to afford 8-(2,2′-dichloro-3′-(4-fluoro-6-methoxy-5-(((((S)-5-oxopyrrolidin-2-yl)methyl)amino) methyl)pyridin-2-yl)-[1,1′-biphenyl]-3-yl)-2-methyl-3-(((((S)-5-oxopyrrolidin-2-yl)methyl) amino)methyl)-4H-pyrido[1,2-a]pyrimidin-4-one (48.3 mg, 19% yield, m/z: 758 [M+H]⁺ observed) as a white solid. ¹H NMR (400 MHz, DMSO-d₆): δ 8.97 (d, J=7.2 Hz, 1H), 7.73-7.51 (m, 8H), 7.45 (d, J=7.6 Hz, 1H), 7.25 (d, J=8.8 Hz, 1H), 4.08 (m, 3H), 4.03-4.01 (m, 2H), 3.69-3.68 (m, 2H), 3.63-3.43 (m, 4H), 3.65-3.64 (m, 1H), 2.60 (d, J=5.6 Hz, 2H), 2.53 (m, 3H), 2.15-2.03 (m, 6H), 1.71-1.65 (m, 2H).

### Example 399: 8-(2,2′-Dichloro-3′-(6-methoxy-5-(((((S)-5-oxopyrrolidin-2-yl)methyl)amino)methyl)pyridin-2-yl)-[1,1′-biphenyl]-3-yl)-2-ethyl-3-(((((S)-5-oxopyrrolidin-2-yl)methyl)amino)methyl)-4H-pyrido[1,2-a]pyrimidin-4-one

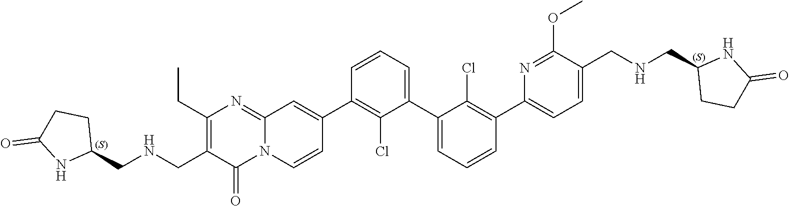

### 2-Chloro-8-(2,2′-dichloro-3′-(5-formyl-6-methoxypyridin-2-yl)-[1,1′-biphenyl]-3-yl)-4-oxo-4H-pyrido[1,2-a]pyrimidine-3-carbaldehyde

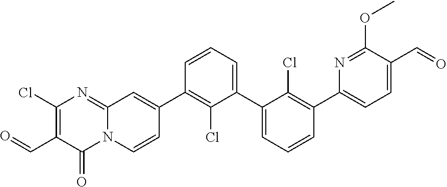 [SMILES: COc1nc(-c2cccc(-c3cccc(-c4ccn5c(=O)c(C=O)c(Cl)nc5c4)c3Cl)c2Cl)ccc1C=O]

To a mixture of 6-(2,2′-dichloro-3′-(2-chloro-3-(hydroxymethyl)-4-oxo-4H-pyrido[1,2-a]pyrimidin-8-yl)-[1,1′-biphenyl]-3-yl)-2-methoxynicotinaldehyde (650 mg, 1.15 mmol) in DCM (10 mL) was added Dess-Martin periodinane (730 mg, 1.7 mmol). The mixture was stirred at rt for 1.5 h. The reaction mixture was treated with water (20 mL) and extracted with EtOAc (3×). The combined organic layers were concentrated under reduced pressure to give a residue. The residue was purified by normal phase SiO₂ chromatography (10-100% in ethyl acetate/petroleum ether) to afford 2-chloro-8-(2,2′-dichloro-3′-(5-formyl-6-methoxypyridin-2-yl)-[1,1′-biphenyl]-3-yl)-4-oxo-4H-pyrido[1,2-a]pyrimidine-3-carbaldehyde (750 mg, 95% yield) as a white solid. ¹H NMR (400 MHz, DMSO-d₆): δ 10.35-10.31 (m, 2H), 9.25 (d, J=7.6 Hz, 1H), 8.26 (d, J=8 Hz, 1H), 8.06 (s, 1H), 7.88-7.85 (m, 1H), 7.83-7.80 (m, 1H), 7.76-7.73 (m, 2H), 7.69-7.66 (m, 2H), 7.62-7.60 (m, 1H), 7.55-7.53 (m, 1H), 4.11 (s, 3H).

### 8-(2,2′-Dichloro-3′-(5-formyl-6-methoxypyridin-2-yl)-[1,1′-biphenyl]-3-yl)-4-oxo-2-vinyl-4H-pyrido[1,2-a]pyrimidine-3-carbaldehyde

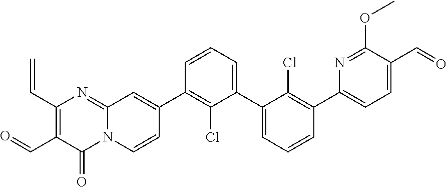 [SMILES: C=Cc1nc2cc(-c3cccc(-c4cccc(-c5ccc(C=O)c(OC)n5)c4Cl)c3Cl)ccn2c(=O)c1C=O]

To a mixture of 2-chloro-8-(2,2′-dichloro-3′-(5-formyl-6-methoxypyridin-2-yl)-[1,1′-biphenyl]-3-yl)-4-oxo-4H-pyrido[1,2-a]pyrimidine-3-carbaldehyde (0.57 g, 1 mmol) and potassium;ethenyl(trifluoro)boranuide (162 mg, 1.2 mmol) in dioxane (9 mL) and water (1 mL) was added 1,1′-bis(diphenylphosphino)ferrocene-palladium(ii) dichloride dichloromethane complex (82.5 mg, 0.1 mmol) and potassium phosphate monobasic (430 mg, 2 mmol). The mixture was stirred at 80° C. for 1 h. The reaction mixture was diluted with water (20 mL) and extracted with EtOAc (50 mL). The organic phase was dried with anhydrous sodium sulfate, filtered and concentrated. The residue was purified by normal phase SiO₂ chromatography (0-100% in ethyl acetate/petroleum ether) to afford 8-(2,2′-dichloro-3′-(5-formyl-6-methoxypyridin-2-yl)-[1,1′-biphenyl]-3-yl)-4-oxo-2-vinyl-4H-pyrido[1,2-a]pyrimidine-3-carbaldehyde (300 mg, 53% yield) as a white solid. ¹H NMR (400 MHz, DMSO-d₆): δ 10.41 (s, 1H), 10.28 (s, 1H), 9.11 (d, J=7.6 Hz, 1H), 8.20 (d, J=7.6 Hz, 1H), 7.874-7.69 (m, 2H), 7.65-7.63 (m, 1H), 7.61-7.59 (m, 5H), 7.56-7.54 (m, 1H), 7.47 (d, J=7.6 Hz, 1H), 6.76-6.71 (m, 1H), 5.88-5.85 (m, 1H), 4.04 (s, 3H).

### 8-(2,2′-Dichloro-3′-(5-formyl-6-methoxypyridin-2-yl)-[1,1′-biphenyl]-3-yl)-2-ethyl-4-oxo-4H-pyrido[1,2-a]pyrimidine-3-carbaldehyde

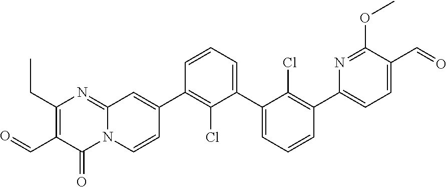 [SMILES: CCc1nc2cc(-c3cccc(-c4cccc(-c5ccc(C=O)c(OC)n5)c4Cl)c3Cl)ccn2c(=O)c1C=O]

To a mixture of 8-(2,2′-dichloro-3′-(5-formyl-6-methoxypyridin-2-yl)-[1,1′-biphenyl]-3-yl)-4-oxo-2-vinyl-4H-pyrido[1,2-a]pyrimidine-3-carbaldehyde (150 mg, 0.27 mmol) in EtOAc (3 mL) was added wet Pd/C (50 mg, 0.54 mmol, 10% purity) under H₂ at 15 psi. The mixture was stirred at rt for 0.5 h. The mixture was filtered through celite and the filtrate was purified by prep-TLC (petroleum ether: ethyl acetate=1:1) to afford 8-(2,2′-dichloro-3′-(5-formyl-6-methoxypyridin-2-yl)-[1,1′-biphenyl]-3-yl)-2-ethyl-4-oxo-4H-pyrido[1,2-a]pyrimidine-3-carbaldehyde (50 mg, 30% yield, m/z: 558 [M+H]⁺, 560 [M+2]⁺ observed) as a yellow solid.

### 8-(2,2′-Dichloro-3′-(6-methoxy-5-(((((S)-5-oxopyrrolidin-2-yl)methyl)amino)methyl)pyridin-2-yl)-[1,1′-biphenyl]-3-yl)-2-ethyl-3-(((((S)-5-oxopyrrolidin-2-yl)methyl)amino)methyl)-4H-pyrido[1,2-a]pyrimidin-4-one

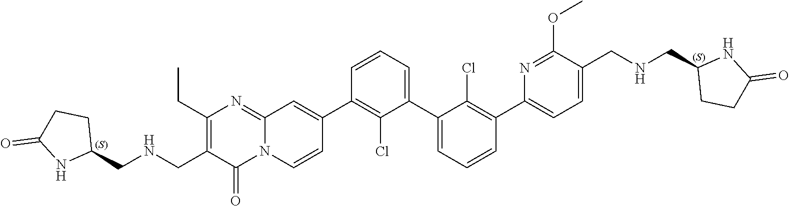

To a mixture of 8-(2,2′-dichloro-3′-(5-formyl-6-methoxypyridin-2-yl)-[1,1′-biphenyl]-3-yl)-2-ethyl-4-oxo-4H-pyrido[1,2-a]pyrimidine-3-carbaldehyde (20 mg, 0.03 mmol) and (S)-5-(aminomethyl)pyrrolidin-2-one, HCl (16 mg, 0.1 mmol) in DCE (1 mL) was added sodium acetate (9 mg, 0.1 mmol) and 4A molecular sieves (50 mg, 0.04 mmol). The mixture was stirred at rt for 12 h followed by stirring at 70° C. for 2 h. Sodium cyanoborohydride (7 mg, 0.18 mmol) and MeOH (1 mL) were added to the mixture and the stirring was continued at rt for 0.5 h. The mixture was concentrated and the residue was purified by reverse phase HPLC to afford 8-(2,2′-dichloro-3′-(6-methoxy-5-(((((S)-5-oxopyrrolidin-2-yl)methyl)amino)methyl)pyridin-2-yl)-[1,1′-biphenyl]-3-yl)-2-ethyl-3-(((((S)-5-oxopyrrolidin-2-yl)methyl)amino)methyl)-4H-pyrido[1,2-a]pyrimidin-4-one (3.4 mg, 12% yield, m/z: 754 [M+H]⁺, 756 [M+2]⁺ observed) as a yellow solid. ¹H NMR (400 MHz, DMSO-d₆): δ 8.96 (d, J=7.6 Hz, 1H), 8.13 (s, 1H), 7.82 (d, J=7.6 Hz, 1H), 7.69-7.68 (m, 4H), 7.67-7.66 (m, 2H), 7.61-7.59 (m, 2H), 7.55-7.44 (m, 2H), 7.27 (d, J=7.2 Hz, 1H), 6.52 (s, 1H), 3.91 (s, 3H), 3.83 (s, 2H), 3.73 (s, 2H), 3.65-3.62 (m, 2H), 2.82 (d, J=7.2 Hz, 2H), 2.60-2.55 (m, 4H), 2.12-2.08 (m, 5H), 1.71-1.64 (m, 2H), 2.25 (t, J=7.6 Hz, 3H).

### Example 376: 8-(2,2′-Dichloro-3′-(6-methoxy-5-(((((S)-5-oxopyrrolidin-2-yl)methyl)amino)methyl)pyridin-2-yl)-[1,1′-biphenyl]-3-yl)-2-((((1r,3r)-3-hydroxy-3-methylcyclobutyl)amino)methyl)-4H-pyrido[1,2-a]pyrimidin-4-one

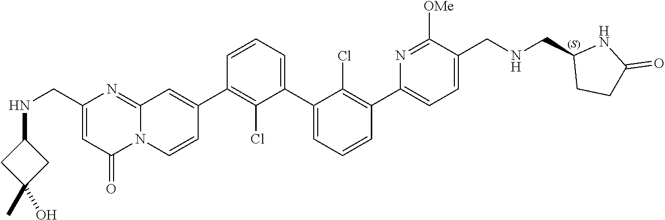

### tert-Butyl ((6-(2,2′-dichloro-3′-(2-((((1r,3r)-3-hydroxy-3-methylcyclobutyl)amino)methyl)-4-oxo-4H-pyrido[1,2-a]pyrimidin-8-yl)-[1,1′-biphenyl]-3-yl)-2-methoxypyridin-3-yl)methyl)(((S)-5-oxopyrrolidin-2-yl)methyl)carbamate

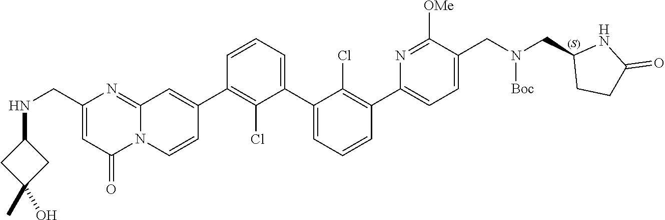

To a mixture of (S)-tert-butyl ((6-(2,2′-dichloro-3′-(2-formyl-4-oxo-4H-pyrido[1,2-a]pyrimidin-8-yl)-[1,1′-biphenyl]-3-yl)-2-methoxypyridin-3-yl)methyl)((5-oxopyrrolidin-2-yl)methyl) carbamate (0.2 g, 0.27 mmol), (1r,3r)-3-amino-1-methylcyclobutanol, HCl (0.06 g, 0.412 mmol), and sodium acetate (0.05 g, 0.68 mmol) in DCM (2 mL) was degassed and purged with N₂, and the mixture was stirred at 25° C. for 11 h under N₂ atmosphere. Sodium cyanoborohydride (0.03 g, 0.54 mmol) was added and the mixture was stirred at 25° C. for 0.5 h under N₂ atmosphere. The reaction mixture was filtered and the filtrate was concentrated under reduced pressure to give a tert-butyl ((6-(2,2′-dichloro-3′-(2-((((1r,3r)-3-hydroxy-3-methylcyclobutyl)amino)methyl)-4-oxo-4H-pyrido[1,2-a]pyrimidin-8-yl)-[1,1′-biphenyl]-3-yl)-2-methoxypyridin-3-yl)methyl)(((S)-5-oxopyrrolidin-2-yl)methyl)carbamate as a yellow solid (0.37 g, crude, m/z:813.1[M+H]⁺ observed).

### 8-(2,2′-Dichloro-3′-(6-methoxy-5-(((((S)-5-oxopyrrolidin-2-yl)methyl)amino)methyl)pyridin-2-yl)-[1,1′-biphenyl]-3-yl)-2-((((1r,3r)-3-hydroxy-3-methylcyclobutyl)amino)methyl)-4H-pyrido[1,2-a]pyrimidin-4-one

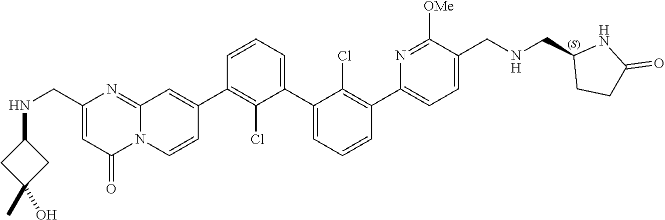

To a mixture of tert-butyl ((6-(2,2′-dichloro-3′-(2-((((1r,3r)-3-hydroxy-3-methylcyclobutyl) amino)methyl)-4-oxo-4H-pyrido[1,2-a]pyrimidin-8-yl)-[1,1′-biphenyl]-3-yl)-2-methoxypyridin-3-yl)methyl)(((S)-5-oxopyrrolidin-2-yl)methyl)carbamate (0.37 g, 0.45 mmol) in DCM (2 mL) was added trifluoroacetic acid (2 mL), and the mixture was stirred at 25° C. for 20 min. The reaction mixture was filtered and concentrated under reduced pressure to give a residue. The residue was purified by reverse phase HPLC to afford 8-(2,2′-dichloro-3′-(6-methoxy-5-(((((S)-5-oxopyrrolidin-2-yl)methyl)amino)methyl)pyridin-2-yl)-[1,1′-biphenyl]-3-yl)-2-((((1r,3r)-3-hydroxy-3-methylcyclobutyl)amino)methyl)-4H-pyrido[1,2-a]pyrimidin-4-one as a white solid (37.1 mg, 10% yield, m/z:713.2[M+H]⁺ observed). ¹H NMR (400 MHz, DMSO-d₆) δ 9.04 (d, J=7.6 Hz, 1H), 7.85 (d, J=7.6 Hz, 1H), 7.77 (s, 1H) 7.70-7.62 (m, 4H), 7.59-7.53 (m, 3H), 7.49 (dd, J=1.6 Hz, J=5.6 Hz, 1H), 7.30 (d, J=7.2 Hz, 1H), 6.55 (s, 1H), 4.92 (s, 1H), 3.93 (m, 5H), 3.82 (m, 2H), 3.69 (s, 2H), 2.67 (m, 2 H), 2.34-2.32 (m, 1H), 2.19-2.09 (m, 5H), 2.00 (m, 2H), 1.72 (m, 1H), 1.27 (s, 3H).

### Example 367: (S)-5-((((6-(2,2′-Dichloro-3′-(8-chloro-6-((((S)-2-hydroxypropyl)amino)methyl)-5-methoxyquinolin-2-yl)-[1,1′-biphenyl]-3-yl)-2-methoxypyridin-3-yl)methyl)amino) methyl)pyrrolidin-2-one

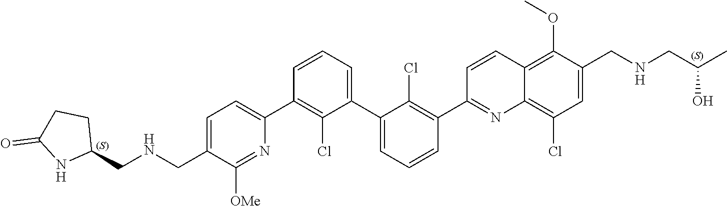

### (S)-tert-Butyl((6-(2,2′-dichloro-3′-(8-chloro-6-formyl-5-methoxyquinolin-2-yl)-[1,1′-biphenyl]-3-yl)-2-methoxypyridin-3-yl)methyl)((5-oxopyrrolidin-2-yl)methyl)carbamate

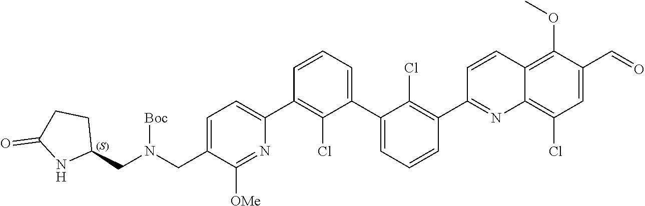

To a mixture of 2,8-dichloro-5-methoxyquinoline-6-carbaldehyde (130 mg, 0.51 mmol) and (S)-tert-butyl ((6-(2,2′-dichloro-3′-(4,4,5,5-tetramethyl-1,3,2-dioxaborolan-2-yl)-[1,1′-biphenyl]-3-yl)-2-methoxypyridin-3-yl)methyl)((5-oxopyrrolidin-2-yl)methyl)carbamate (520 mg, 0.76 mmol) in dioxane/water mixture (5:1, 12.5 mL) was added potassium carbonate (210 mg, 1.52 mmol), [1,1-bis(diphenylphosphino)ferrocene]palladium(II) chloride dichloromethane complex (41.5 mg, 0.05 mmol) and the mixture was stirred at 110° C. for 0.5 h under N₂. The mixture was filtered and the filtrate was concentrated to give the residue. The residue was purified by normal phase SiO₂ chromatography (0-80% EtOAc/petroleum ether) to afford (S)-tert-butyl((6-(2,2′-dichloro-3′-(8-chloro-6-formyl-5-methoxyquinolin-2-yl)-[1,1′-biphenyl]-3-yl)-2-methoxypyridin-3-yl)methyl)((5-oxopyrrolidin-2-yl)methyl)carbamate (400 mg, crude, m/z: 775 [M+H]⁺ observed) as a yellow solid.

### tert-Butyl((6-(2,2′-dichloro-3′-(8-chloro-6-((((S)-2-hydroxypropyl)amino)methyl)-5-methoxyquinolin-2-yl)-[1,1′-biphenyl]-3-yl)-2-methoxypyridin-3-yl)methyl)(((S)-5-oxopyrrolidin-2-yl)methyl)carbamate

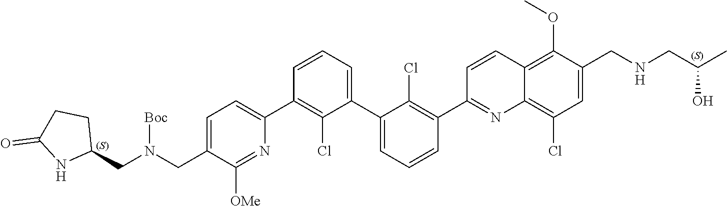

To a mixture of (S)-tert-butyl ((6-(2,2′-dichloro-3′-(8-chloro-6-formyl-5-methoxyquinolin-2-yl)-[1,1′-biphenyl]-3-yl)-2-methoxypyridin-3-yl)methyl)((5-oxopyrrolidin-2-yl)methyl)carbamate (120 mg, 0.15 mmol) and (S)-1-aminopropan-2-ol (35 mg, 0.46 mmol) in DCM (10 mL) was added sodium acetate (38 mg, 0.46 mmol), and stirred at rt for 12 h under N₂. Sodium cyanoborohydride (29 mg, 0.46 mmol) was added to the mixture and stirred at rt for 0.5 h under N₂. The mixture was concentrated to give the residue. The residue was purified by prep-TLC (SiO₂, EtOAc/MeOH=9:1) to afford tert-butyl((6-(2,2′-dichloro-3′-(8-chloro-6-((((S)-2-hydroxypropyl)amino)methyl)-5-methoxyquinolin-2-yl)-[1,1′-biphenyl]-3-yl)-2-methoxypyridin-3-yl)methyl)(((S)-5-oxopyrrolidin-2-yl)methyl)carbamate (100 mg, 77% yield, m/z: 834 [M+H]⁺ observed) as a yellow oil.

### (S)-5-((((6-(2,2′-Dichloro-3′-(8-chloro-6-((((S)-2-hydroxypropyl)amino)methyl)-5-methoxyquinolin-2-yl)-[1,1′-biphenyl]-3-yl)-2-methoxypyridin-3-yl)methyl)amino) methyl)pyrrolidin-2-one

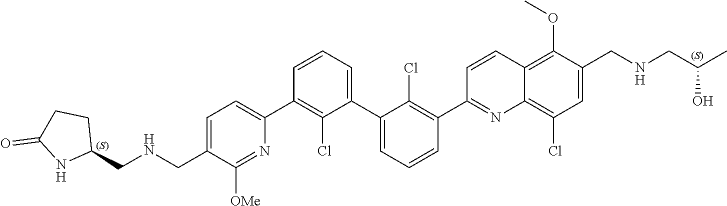

To a mixture of tert-butyl ((6-(2,2′-dichloro-3′-(8-chloro-6-((((S)-2-hydroxypropyl)amino)methyl)-5-methoxyquinolin-2-yl)-[1,1′-biphenyl]-3-yl)-2-methoxypyridin-3-yl)methyl)(((S)-5-oxopyrrolidin-2-yl)methyl)carbamate (100 mg, 0.12 mmol) in DCM (5 mL) was added trifluoroacetic acid (2 mL, 27.01 mmol). The mixture was stirred at rt for 0.5 h. Then the mixture was concentrated. The residue was purified by reverse phase HPLC to afford (S)-5-((((6-(2,2′-dichloro-3′-(8-chloro-6-((((S)-2-hydroxypropyl)amino)methyl)-5-methoxyquinolin-2-yl)-[1,1′-biphenyl]-3-yl)-2-methoxypyridin-3-yl)methyl)amino)methyl) pyrrolidin-2-one (14.2 mg, m/z: 734 [M+H]⁺ observed) as a white solid. ¹H NMR (400 MHz, DMSO-d₆): δ 8.64 (d, J=8.8 Hz, 1H), 7.96 (d, J=8.8 Hz, 1H), 7.85 (d, J=7.6 Hz, 1H), 7.76-7.66 (m, 4H), 7.60-7.59 (m, 2H), 7.53-7.52 (m, 1H), 7.31 (d, J=7.2 Hz, 1H), 4.67 (s, 1H), 4.07-4.05 (m, 2H), 3.97 (s, 3H), 3.94 (s, 3H), 3.79-3.75 (m, 4H), 3.68-3.65 (m, 2H), 3.34-3.32 (m, 2H), 2.68-2.67 (m, 2H), 2.15-2.09 (m, 3H), 1.73-1.71 (m, 1H), 1.08 (d, J=6 Hz, 1H).

### Example 371: (S)-8-(2,2′-Dichloro-3′-(6-methoxy-5-((((5-oxopyrrolidin-2-yl)methyl)amino)methyl)pyridin-2-yl)-[1,1′-biphenyl]-3-yl)-2-(((2-hydroxyethyl)amino)methyl)-3-methyl-4H-pyrido[1,2-a]pyrimidin-4-one

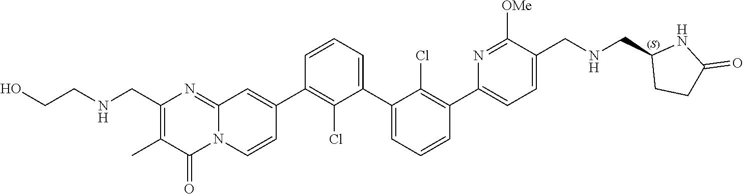

### Ethyl 4-bromo-2-methyl-3-oxobutanoate

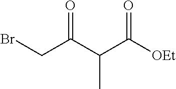

To a cooled (0° C.) solution of ethyl 2-methyl-3-oxobutanoate (9.80 mL, 69.4 mmol) in CHCl₃ (65 ml), was added a solution of bromine (3.58 mL, 69.4 mmol) in CHCl₃ (5 ml). After complete addition, the mixture was allowed to warm to 20° C. and stirred for 12 h. The solution was dried over anhydrous sodium sulfate, filtered and evaporated to give the crude ethyl 4-bromo-2-methyl-3-oxobutanoate as an orange oil (15.53 g, crude), which was used into the next step without further purification.

### 8-Bromo-2-(bromomethyl)-3-methyl-4H-pyrido[1,2-a]pyrimidin-4-one

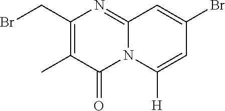

The mixture of 4-bromopyridin-2-amine (5 g, 28.90 mmol) and ethyl 4-bromo-2-methyl-3-oxobutanoate (12.9 g, 57.8 mmol) in PPA (100 mL) was stirred for 0.5 h at 110° C. After cooling to 0° C., the reaction was quenched with water (200 mL) and stirred for 1 h. Th aqueous layer was extracted with EtOAc (3×), separated, dried over sodium sulfate and concentrated. The residue was purified by normal phase SiO₂ chromatography (0-20% EtOAc/petroleum ether) to afford 8-bromo-2-(bromomethyl)-3-methyl-4H-pyrido[1,2-a]pyrimidin-4-one as a white solid (0.9 g, 9.38% yield). 1H NMR (400 MHz, CDCl₃): δ 8.85 (d, J=7.6 Hz, 1H), 7.74 (d, J=1.6 Hz, 1H), 7.09 (dd, J=2.0 Hz, J=7.6 Hz, 1H), 4.37 (s, 2H), 2.24 (s, 3H).

### 8-Bromo-2-(((2-hydroxyethyl)amino)methyl)-3-methyl-4H-pyrido[1,2-a]pyrimidin-4-one

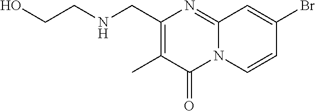 [SMILES: Cc1c(CNCCO)nc2cc(Br)ccn2c1=O]

To a mixture of 8-bromo-2-(bromomethyl)-3-methyl-4H-pyrido[1,2-a]pyrimidin-4-one (600 mg, 1.81 mmol) in DMF (5 mL) was added 2-aminoethanol (0.55 mL, 9.04 mmol) and potassium carbonate (749 mg, 5.42 mmol). The reaction was stirred for 2 h at rt. To the reaction was added water (30 mL) and extracted with EtOAc (300 mL). The organic layer was concentrated in vacuo to afford the crude 8-bromo-2-[(2-hydroxyethylamino)methyl]-3-methyl-pyrido[1,2-a]pyrimidin-4-one as an orange oil (564 mg, crude), which was used into the next step without further purification.

### tert-Butyl ((8-bromo-3-methyl-4-oxo-4H-pyrido[1,2-a]pyrimidin-2-yl)methyl)(2-hydroxyethyl)carbamate

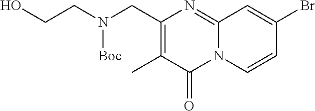 [SMILES: Cc1c(CN(CCO)C(=O)OC(C)(C)C)nc2cc(Br)ccn2c1=O]

To a mixture of 8-bromo-2-(((2-hydroxyethyl)amino)methyl)-3-methyl-4H-pyrido[1,2-a]pyrimidin-4-one (564 mg, 1.81 mmol) in DCM (10 mL) was added di-tert-butyl dicarbonate (591 mg, 2.71 mmol) and TEA (0.75 ml, 5.42 mmol). The reaction was stirred for 12 h at rt. The reaction was concentrated in vacuo. The residue was purified by normal phase SiO₂ chromatography (0-100% EtOAc/petroleum ether) to afford tert-butyl ((8-bromo-3-methyl-4-oxo-4H-pyrido[1,2-a]pyrimidin-2-yl)methyl)(2-hydroxyethyl)carbamate as a yellow oil (540 mg, 1.11 mmol, 61.62% yield, 85% purity), m/z: 412 [M]⁺, 414 [M+2]⁺ observed. ¹H NMR (400 MHz, CDCl₃): δ 8.85 (dd, J=7.6 Hz, J=12.4 Hz, 1H), 8.04 (s, 1H), 7.76 (d, J=22 Hz, 1H), 4.47 (d, J=6.8 Hz, 2H), 3.89-3.82 (m, 2H), 3.64-3.59 (m, 2H), 2.32-2.30 (m, 3 H), 1.62 (s, 9H).

### (S)-tert-Butyl ((6-(3′-(2-(((tert-butoxycarbonyl)(2-hydroxyethyl)amino)methyl)-3-methyl-4-oxo-4H-pyrido[1,2-a]pyrimidin-8-yl)-2,2′-dichloro-[1,1′-biphenyl]-3-yl)-2-methoxypyridin-3-yl)methyl)((5-oxopyrrolidin-2-yl)methyl)carbamate

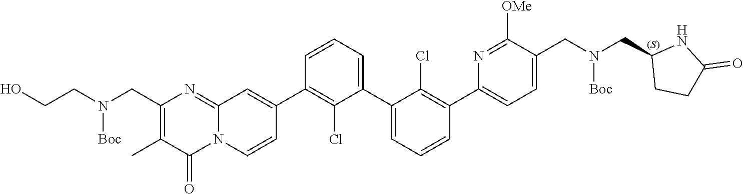

To a mixture of tert-butyl ((8-bromo-3-methyl-4-oxo-4H-pyrido[1,2-a]pyrimidin-2-yl) methyl)(2-hydroxyethyl)carbamate (510 mg, 1.24 mmol) and (S)-tert-butyl ((6-(2,2′-dichloro-3′-(4,4,5,5-tetramethyl-1,3,2-dioxaborolan-2-yl)-[1,1′-biphenyl]-3-yl)-2-methoxypyridin-3-yl)methyl)((5-oxopyrrolidin-2-yl)methyl)carbamate (1.01 g, 1.48 mmol) in dioxane/water (10/1, 22 mL) was added 1,1′-bis(diphenylphosphino)ferrocene-palladium(II)dichloride dichloromethane complex (101 mg, 0.12 mmol), followed by potassium phosphate (263 mg, 1.24 mmol) and was stirred for 1 h at 80° C. After cooling, the mixture was diluted with water (50 mL) and extracted with EtOAc (100 mL). The organic layer was dried over anhydrous sodium sulfate, filtered and concentrated in vacuo. The residue was purified by normal phase SiO₂ chromatography (0-100% EtOAc/30% THE in petroleum ether) to afford (S)-tert-butyl ((6-(3′-(2-(((tert-butoxycarbonyl)(2-hydroxyethyl)amino)methyl)-3-methyl-4-oxo-4H-pyrido[1,2-a]pyrimidin-8-yl)-2,2′-dichloro-[1,1′-biphenyl]-3-yl)-2-methoxypyridin-3-yl)methyl)((5-oxopyrrolidin-2-yl)methyl)carbamate as a brown solid (0.41 g, 31% yield, 84% purity). m/z: 887 [M]⁺, 889 [M+2]⁺ observed. ¹H NMR (400 MHz, Methanol-d₄): δ 9.07 (t, J=6.4 Hz, 1H), 7.67 (dd, J=2.0 Hz, J=7.6 Hz, 2H), 7.62-7.48 (m, 6H), 7.40 (dd, J=2.0 Hz, J=7.6 Hz, 1H), 7.27 (d, J=7.6 Hz, 1H), 4.63 (s, 2H), 4.55 (s, 2H), 4.03 (s, 3H), 3.99-3.96 (m, 1H), 3.79-3.78 (m, 2 H), 3.59-3.57 (m, 2H), 3.50-3.41 (m, 2H), 3.38-3.34 (m, 2H), 2.40-2.23 (m, 5H), 1.49-1.24 (m, 18H).

### (S)-8-(2,2′-Dichloro-3′-(6-methoxy-5-((((5-oxopyrrolidin-2-yl)methyl)amino)methyl)pyridin-2-yl)-[1,1′-biphenyl]-3-yl)-2-(((2-hydroxyethyl)amino)methyl)-3-methyl-4H-pyrido[1,2-a]pyrimidin-4-one

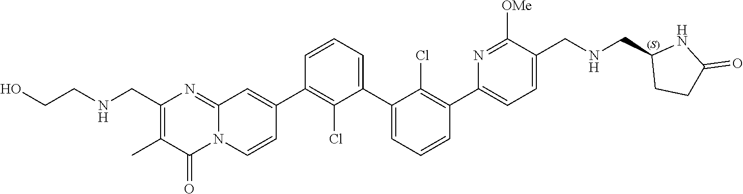

To a solution of (S)-tert-butyl ((6-(3′-(2-(((tert-butoxycarbonyl)(2-hydroxyethyl) amino)methyl)-3-methyl-4-oxo-4H-pyrido[1,2-a]pyrimidin-8-yl)-2,2′-dichloro-[1,1′-biphenyl]-3-yl)-2-methoxypyridin-3-yl)methyl)((5-oxopyrrolidin-2-yl)methyl)carbamate (0.4 g, 0.45 mmol) in DCM (8 mL) was added 2,2,2-trifluoroacetic acid (1.95 mL, 26.4 mmol) and stirred for 0.5 h at rt. The mixture was concentrated, and the residue was purified by reverse phase HPLC to afford (S)-8-(2,2′-dichloro-3′-(6-methoxy-5-((((5-oxopyrrolidin-2-yl) methyl)amino)methyl)pyridin-2-yl)-[1,1′-biphenyl]-3-yl)-2-(((2-hydroxy ethyl)amino)methyl)-3-methyl-4H-pyrido[1,2-a]pyrimidin-4-one as a yellow solid (87 mg, 27.8% yield). m/z: 687 [M]⁺, 689 [M+2]⁺ observed. ¹H NMR (400 MHz, DMSO-d₆): δ 8.99 (d, J=7.2 Hz, 1H), 8.22 (s, 1H), 7.84 (d, J=7.6 Hz, 1H), 7.73-7.62 (m, 4H), 7.58-7.52 (m, 4H), 7.28 (d, J=7.6 Hz, 1H), 4.05 (s, 1H), 3.93 (s, 3H), 3.74 (s, 3H), 3.70-3.59 (m, 4H), 2.90-2.87 (m, 2H), 2.58 (d, J=6.0 Hz, 2H), 2.19 (s, 2H), 2.15-2.09 (m, 3H), 1.73-1.69 (m, 1H).

### Example 372: 8-(2,2′-Dichloro-3′-(6-methoxy-5-(((((S)-5-oxopyrrolidin-2-yl)methyl)amino)methyl)pyridin-2-yl)-[1,1′-biphenyl]-3-yl)-3-methyl-2-(((((S)-5-oxopyrrolidin-2-yl)methyl)amino)methyl)-4H-pyrido[1,2-a]pyrimidin-4-one

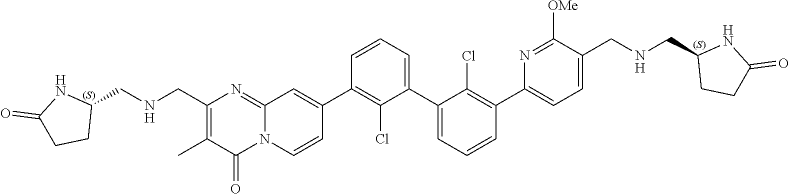

### (S)-8-Bromo-3-methyl-2-((((5-oxopyrrolidin-2-yl)methyl)amino)methyl)-4H-pyrido[1,2-a]pyrimidin-4-one

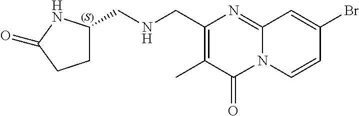

To a mixture of 8-bromo-2-(bromomethyl)-3-methyl-4H-pyrido[1,2-a]pyrimidin-4-one (500 mg, 1.51 mmol) in DMF (10 mL) was added (S)-5-(aminomethyl)pyrrolidin-2-one, HCl (860 mg, 7.53 mmol) and potassium carbonate (624 mg, 4.52 mmol). The reaction was stirred for 2 h at rt. Compound (S)-8-bromo-3-methyl-2-((((5-oxopyrrolidin-2-yl)methyl)amino)methyl)-4H-pyrido[1,2-a]pyrimidin-4-one was obtained as a DMF solution (10 mL). The crude product was used directly without further purification.

### (S)-tert-butyl ((8-bromo-3-methyl-4-oxo-4H-pyrido[1,2-a]pyrimidin-2-yl)methyl)((5-oxopyrrolidin-2-yl)methyl)carbamate

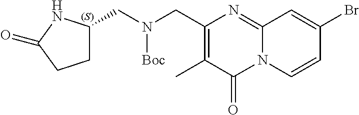

To a mixture of (S)-8-bromo-3-methyl-2-((((5-oxopyrrolidin-2-yl)methyl)amino) methyl)-4H-pyrido[1,2-a]pyrimidin-4-one (550 mg, 1.51 mmol) in DMF (10 mL) was added di-tert-butyl dicarbonate (2.77 mL, 12.05 mmol) and TEA (0.63 mL, 4.52 mmol). The reaction was stirred at rt for 12 h. The reaction was concentrated in vacuo. The residue was purified by normal phase SiO₂ chromatography (0-100% EtOAc/petroleum ether) to afford (S)-tert-butyl ((8-bromo-3-methyl-4-oxo-4H-pyrido[1,2-a]pyrimidin-2-yl) methyl) ((5-oxopyrrolidin-2-yl)methyl)carbamate as a yellow oil (720 mg, 98.18% yield). ¹H NMR (400 MHz, CDCl₃): δ 7.95 (s, 1H), 6.76 (s, 1H), 5.23 (s, 1H), 3.72 (s, 2H), 3.24-3.23 (s, 2H), 3.06-2.99 (m, 1H), 2.89 (s, 3H), 2.16-2.11 (m, 2H), 1.76-1.67 (m, 2H), 1.37 (s, 9H).

### tert-Butyl ((6-(3′-(2-(((tert-butoxycarbonyl)(((R)-5-oxopyrrolidin-2-yl)methyl)amino) methyl)-3-methyl-4-oxo-4H-pyrido[1,2-a]pyrimidin-8-yl)-2,2′-dichloro-[1,1′-biphenyl]-3-yl)-2-methoxypyridin-3-yl)methyl)(((S)-5-oxopyrrolidin-2-yl)methyl)carbamate

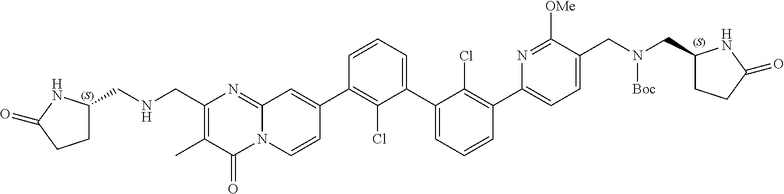

To a mixture of (S)-8-bromo-3-methyl-2-((methyl(5-oxopyrrolidin-2-yl)amino) methyl)-4H-pyrido[1,2-a]pyrimidin-4-one (480 mg, 1.31 mmol) and (S)-tert-butyl ((6-(2,2′-dichloro-3′-(4,4,5,5-tetramethyl-1,3,2-dioxaborolan-2-yl)-[1,1′-biphenyl]-3-yl)-2-methoxypyridin-3-yl)methyl)((5-oxopyrrolidin-2-yl)methyl)carbamate (718 mg, 1.05 mmol) in THF/water (10/1, 22 mL) was added potassium carbonate (545 mg, 3.94 mmol) and tetrakis(triphenylphosphine) palladium(0) (75.9 mg, 0.07 mmol). The reaction was stirred for 1 h at 80° C. After cooling, the mixture was diluted with water (20 mL) and extracted with EtOAc (30 mL). The organic layer was dried over anhydrous sodium sulfate, filtered and concentrated in vacuo. The residue was purified by normal phase SiO₂ chromatography (0-100% EtOAc/Petroleum ether gradient to 0-20% MeOH/EtOAc) to afford tert-butyl ((6-(3′-(2-(((tert-butoxycarbonyl)(((R)-5-oxopyrrolidin-2-yl) methyl)amino)methyl)-3-methyl-4-oxo-4H-pyrido[1,2-a]pyrimidin-8-yl)-2,2′-dichloro-[1,1′-biphenyl]-3-yl)-2-methoxypyridin-3-yl)methyl)(((S)-5-oxopyrrolidin-2-yl)methyl) carbamate as a yellow solid (0.54 g, 0.49 mmol).

### 8-(2,2′-Dichloro-3′-(6-methoxy-5-(((((S)-5-oxopyrrolidin-2-yl)methyl)amino) methyl) pyridin-2-yl)-[1,1′-biphenyl]-3-yl)-3-methyl-2-(((((S)-5-oxopyrrolidin-2-yl)methyl)amino) methyl)-4H-pyrido[1,2-a]pyrimidin-4-one

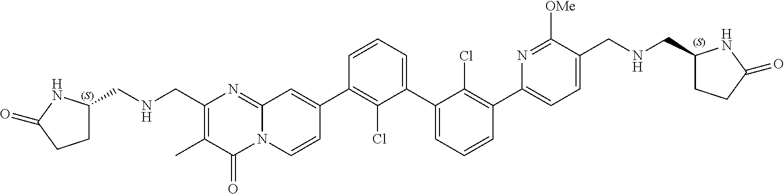

To a solution of tert-butyl ((6-(3′-(2-(((tert-butoxycarbonyl)(((R)-5-oxopyrrolidin-2-yl) methyl)amino)methyl)-3-methyl-4-oxo-4H-pyrido[1,2-a]pyrimidin-8-yl)-2,2′-dichloro-[1,1′-biphenyl]-3-yl)-2-methoxypyridin-3-yl)methyl)(((S)-5-oxopyrrolidin-2-yl)methyl)carbamate (0.53 g, 0.56 mmol) in DCM (1 mL) was added 2,2,2-trifluoroacetic acid (5.30 mL, 71.58 mmol) and stirred for 0.5 h at rt. The mixture was concentrated in vacuo and the residue was purified by reverse phase HPLC to afford 8-(2,2′-dichloro-3′-(6-methoxy-5-(((((S)-5-oxopyrrolidin-2-yl)methyl)amino)methyl)pyridin-2-yl)-[1,1′-biphenyl]-3-yl)-3-methyl-2-(((((S)-5-oxopyrrolidin-2-yl)methyl)amino)methyl)-4H-pyrido[1,2-a]pyrimidin-4-one as a yellow solid (105.5 mg, 23% yield). m/z: 740 [M]⁺, 742 [M+2]⁺ observed. ¹H NMR (400 MHz, DMSO-d₆): δ 8.97 (d, J=7.6 Hz, 1H), 8.17 (s, 1H), 7.85 (d, J=7.6 Hz, 1H), 7.73-7.62 (m, 6H), 7.58-7.55 (m, 2H), 7.49-7.44 (m, 2H), 7.29 (d, J=7.6 Hz, 1H), 3.90 (s, 3H), 3.76 (s, 3H), 3.70-3.66 (m, 3H), 2.67 (d, J=6.0 Hz, 2H), 2.61 (d, J=6.0 Hz, 2H), 2.20 (s, 3H), 2.17-2.07 (m, 6H), 1.76-1.70 (m, 2H).

### Example 377: 8-(2,2′-Dichloro-3′-(6-methoxy-5-(((((S)-5-oxopyrrolidin-2-yl)methyl)amino)methyl)pyridin-2-yl)-[1,1′-biphenyl]-3-yl)-2-((((S)-2-hydroxypropyl)amino)methyl)-3-methyl-4H-pyrido[1,2-a]pyrimidin-4-one

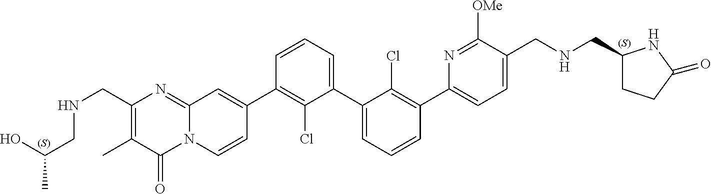

### (S)-tert-Butyl ((8-bromo-3-methyl-4-oxo-4H-pyrido[1,2-a]pyrimidin-2-yl)methyl)(2-hydroxypropyl)carbamate

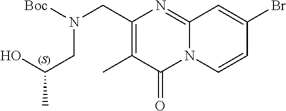

To a solution of 8-bromo-2-(bromomethyl)-3-methyl-4H-pyrido[1,2-a]pyrimidin-4-one (0.5 g, 1.51 mmol) in DMF (50 mL) was added (S)-1-aminopropan-2-ol (0.56 g, 7.53 mmol) and potassium carbonate (0.62 g, 4.52 mmol). The mixture was purged with N₂ and stirred at 25° C. for 1 h. Compound (S)-8-bromo-2-(((2-hydroxypropyl)amino)methyl)-3-methyl-4H-pyrido[1,2-a]pyrimidin-4-one was obtained as a yellow solution (in DMF) (m/z: 326.0 [M+H]⁺ observed). To this solution of (S)-8-bromo-2-(((2-hydroxypropyl)amino)methyl)-3-methyl-4H-pyrido[1,2-a]pyrimidin-4-one was added di-tert-butyl dicarbonate (1.64 g, 7.53 mmol) and TEA (0.30 g, 3.01 mmol). The mixture was stirred at 25° C. for 0.5 h. The reaction mixture was diluted with water (50 mL) and extracted with EtOAc (3×30 mL). The combined organic layers were washed with brine (20 mL), dried over anhydrous sodium sulfate, filtered and concentrated under reduced pressure. The residue was purified by normal phase SiO₂ chromatography (0-100% EtOAc/petroleum ether) to afford (S)-tert-butyl ((8-bromo-3-methyl-4-oxo-4H-pyrido[1,2-a]pyrimidin-2-yl)methyl)(2-hydroxypropyl)carbamate was obtained as yellow oily liquid (1 g, crude, m/z: 426.0[M+H]⁺ observed).

### tert-Butyl ((6-(3′-(2-(((tert-butoxycarbonyl)((S)-2-hydroxypropyl)amino)methyl)-3-methyl-4-oxo-4H-pyrido[1,2-a]pyrimidin-8-yl)-2,2′-dichloro-[1,1′-biphenyl]-3-yl)-2-methoxypyridin-3-yl)methyl)(((S)-5-oxopyrrolidin-2-yl)methyl)carbamate

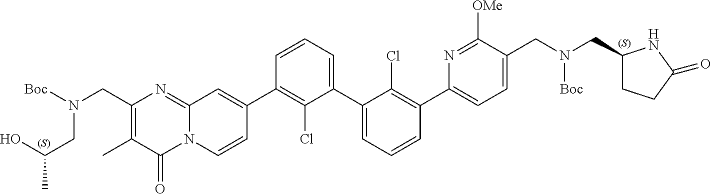

To a mixture of (S)-tert-butyl ((8-bromo-3-methyl-4-oxo-4H-pyrido[1,2-a]pyrimidin-2-yl)methyl)(2-hydroxypropyl)carbamate(0.5 g, 1.15 mmol) and (S)-tert-butyl ((6-(2,2′-dichloro-3′-(4,4,5,5-tetramethyl-1,3,2-dioxaborolan-2-yl)-[1,1′-biphenyl]-3-yl)-2-methoxypyridin-3-yl)methyl)((5-oxopyrrolidin-2-yl)methyl)carbamate (4 g, 0.55 mmol), and potassium phosphate (0.6 g, 2.9 mmol) in dioxane (5 mL) and water (0.5 mL) was added [1,1′-bis(di-tert-butylphosphino)ferrocene]dichloropalladium(II) (0.05 g, 0.1 mmol). The reaction was degassed and purged with N₂, and stirred at 80° C. for 1.5 h. The reaction was diluted with water (50 mL) and extracted with EtOAc (3×10 mL). The combined organic layers were washed with brine (2×10 mL), dried over anhydrous sodium sulfate, filtered and concentrated under reduced pressure. The residue was purified by normal phase SiO₂ chromatography (0-100% EtOAc/petroleum ether) to afford tert-butyl ((6-(3′-(2-(((tert-butoxycarbonyl)((S)-2-hydroxypropyl)amino)methyl)-3-methyl-4-oxo-4H-pyrido[1,2-a]pyrimidin-8-yl)-2,2′-dichloro-[1,1′-biphenyl]-3-yl)-2-methoxypyridin-3-yl)methyl)(((S)-5-oxopyrrolidin-2-yl)methyl) carbamate as a yellow solid (0.14 g, m/z: 901.2 [M+H]⁺ observed).

### 8-(2,2′-Dichloro-3′-(6-methoxy-5-(((((S)-5-oxopyrrolidin-2-yl)methyl)amino)methyl)pyridin-2-yl)-[1,1′-biphenyl]-3-yl)-2-((((S)-2-hydroxypropyl)amino)methyl)-3-methyl-4H-pyrido[1,2-a]pyrimidin-4-one

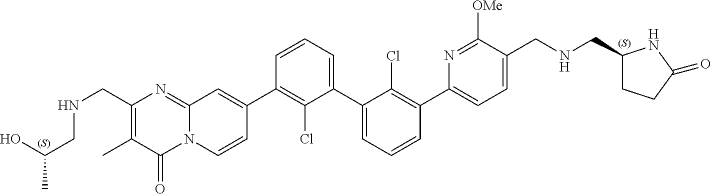

To a mixture of tert-butyl ((6-(3′-(2-(((tert-butoxycarbonyl)((S)-2-hydroxypropyl) amino)methyl)-3-methyl-4-oxo-4H-pyrido[1,2-a]pyrimidin-8-yl)-2,2′-dichloro-[1,1′-biphenyl]-3-yl)-2-methoxypyridin-3-yl)methyl)(((S)-5-oxopyrrolidin-2-yl)methyl) carbamate (0.14 g, 0.14 mmol) in DCM (2 mL) was added trifluoroacetic acid (0.6 mL), and stirred at 25° C. for 15 min. The mixture was filtered and concentrated under reduced pressure to give a residue. The residue was purified by reverse phase HPLC to afford 8-(2,2′-dichloro-3′-(6-methoxy-5-(((((S)-5-oxopyrrolidin-2-yl)methyl)amino) methyl)pyridin-2-yl)-[1,1′-biphenyl]-3-yl)-2-((((S)-2-hydroxypropyl)amino)methyl)-3-methyl-4H-pyrido[1,2-a]pyrimidin-4-one as a white solid (25.4 mg, m/z: 701.1 [M+H]⁺ observed). ¹H NMR (400 MHz, DMSO-d₆) δ 8.98 (d, J=7.6 Hz, 1H), 7.84 (d, J=7.6 Hz, 1H), 7.72-7.68 (m, 4H), 7.64 (t, J=7.4 Hz, 1H), 7.60-7.56 (m, 2H), 7.50-7.44 (m, 2H), 7.29 (d, J=7.6 Hz, 1H), 3.92-3.64 (m, 9H), 2.65-2.57 (m, 3H), 2.21-2.08 (m, 7H), 1.76-1.67 (m, 1H), 1.07 (d, J=6.0 Hz, 3H).

### Example 373: 8-(2,2′-Dichloro-3′-(6-methoxy-5-(((((S)-5-oxopyrrolidin-2-yl)methyl)amino)methyl)pyridin-2-yl)-[1,1′-biphenyl]-3-yl)-2-((((1r,3r)-3-hydroxy-3-methylcyclobutyl)amino)methyl)-3-methyl-4H-pyrido[1,2-a]pyrimidin-4-one

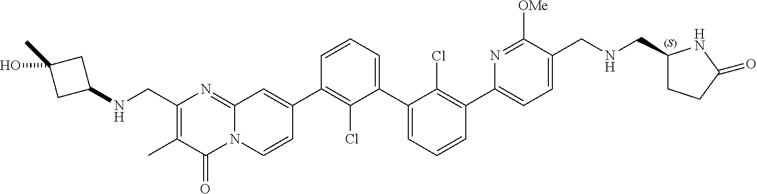

### 8-Bromo-2-((((1r,3r)-3-hydroxy-3-methylcyclobutyl)amino)methyl)-3-methyl-4H-pyrido[1,2-a]pyrimidin-4-one

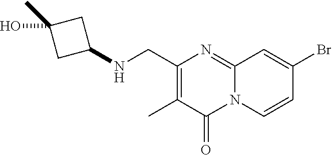

To a mixture of (1r,3r)-3-amino-1-methylcyclobutanol as a hydrochloride salt (259 mg, 1.88 mmol) and potassium carbonate (312 mg, 2.26 mmol) in DMF (10 ml) was added the solution of 8-bromo-2-(bromomethyl)-3-methyl-4H-pyrido[1,2-a]pyrimidin-4-one (250 mg, 0.75 mmol) in DMF (4 mL) slowly. The reaction was stirred for 2 h at rt. Compound 8-bromo-2-((((1r,3r)-3-hydroxy-3-methylcyclobutyl)amino)methyl)-3-methyl-4H-pyrido[1,2-a]pyrimidin-4-one (265 mg, crude) was obtained as a DMF solution (14 mL). The crude product was used directly without any purification.

### tert-Butyl ((8-bromo-3-methyl-4-oxo-4H-pyrido[1,2-a]pyrimidin-2-yl)methyl)((1r,3r)-3-hydroxy-3-methylcyclobutyl)carbamate

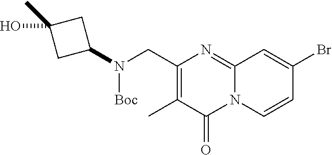

To a solution of 8-bromo-2-((((1r,3r)-3-hydroxy-3-methylcyclobutyl)amino)methyl)-3-methyl-4H-pyrido[1,2-a]pyrimidin-4-one (265 mg, 0.75 mmol) in DMF (14 mL) was added di-tert-butyl dicarbonate (821 mg, 3.76 mmol) and TEA (228 mg, 2.26 mmol). The reaction was stirred for 12 h at rt. The reaction was concentrated in vacuo and the residue was purified by normal phase SiO₂ chromatography (0-100% EtOAc/petroleum ether) to afford tert-butyl ((8-bromo-3-methyl-4-oxo-4H-pyrido[1,2-a]pyrimidin-2-yl) methyl)((1r,3r)-3-hydroxy-3-methylcyclobutyl) carbamate as a yellow oil (0.5 g, 96% yield). m/z: 452 [M]⁺, 454 [M+2]⁺ observed.

### tert-Butyl ((6-(3′-(2-(((tert-butoxycarbonyl)((1r,3r)-3-hydroxy-3-methylcyclobutyl)amino) methyl)-3-methyl-4-oxo-4H-pyrido[1,2-a]pyrimidin-8-yl)-2,2′-dichloro-[1,1′-biphenyl]-3-yl)-2-methoxypyridin-3-yl)methyl)(((S)-5-oxopyrrolidin-2-yl)methyl)carbamate

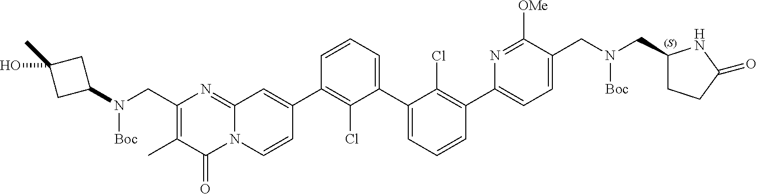

To a mixture of tert-butyl ((8-bromo-3-methyl-4-oxo-4H-pyrido[1,2-a]pyrimidin-2-yl) methyl)((1r,3r)-3-hydroxy-3-methylcyclobutyl)carbamate (470 mg, 1.04 mmol) and (S)-tert-butyl ((6-(2,2′-dichloro-3′-(4,4,5,5-tetramethyl-1,3,2-dioxaborolan-2-yl)-[1,1′-biphenyl]-3-yl)-2-methoxypyridin-3-yl)methyl)((5-oxopyrrolidin-2-yl)methyl)carbamate (780 mg, 1.14 mmol) in THF/water (10/1, 22 mL) was added tetrakis(triphenylphosphine)palladium(0) (60 mg, 0.052 mmol) and potassium carbonate (431 mg, 3.12 mmol). The reaction was stirred for 1 h at 80° C. After cooling, the mixture was diluted with water (20 mL) and extracted with EtOAc (80 mL). The organic layer was dried over anhydrous sodium sulfate, filtered and concentrated in vacuo. The residue was purified by normal phase SiO₂ chromatography (0-100% EtOAc/30% THF in petroleum ether) to afford tert-butyl ((6-(3′-(2-(((tert-butoxycarbonyl)((1r,3r)-3-hydroxy-3-methylcyclobutyl)amino)methyl)-3-methyl-4-oxo-4H-pyrido[1,2-a]pyrimidin-8-yl)-2,2′-dichloro-[1,1′-biphenyl]-3-yl)-2-methoxypyridin-3-yl)methyl)(((S)-5-oxopyrrolidin-2-yl)methyl)carbamate (0.26 g, 20% yield). ¹H NMR (400 MHz, CDCl₃): δ 9.05 (d, J=7.2 Hz, 1H), 7.68 (d, J=7.6 Hz, 2H), 7.46-7.44 (m, 6H), 7.44-7.40 (m, 2H), 7.28 (d, J=7.2 Hz, 1H), 4.62-4.55 (m, 5H), 4.03 (s, 3H), 3.99-3.97 (m, 1H), 3.76-3.73 (m, 4H), 3.47-3.45 (m, 2H), 2.38-2.24 (m, 7H), 2.03 (s, 2H), 1.91-1.88 (m, 4H), 1.53 (be s, 4H), 1.42 (s, 18H), 1.31 (s, 3H).

### 8-(2,2′-Dichloro-3′-(6-methoxy-5-(((((S)-5-oxopyrrolidin-2-yl)methyl)amino)methyl)pyridin-2-yl)-[1,1′-biphenyl]-3-yl)-2-((((1r,3r)-3-hydroxy-3-methylcyclobutyl)amino)methyl)-3-methyl-4H-pyrido[1,2-a]pyrimidin-4-one

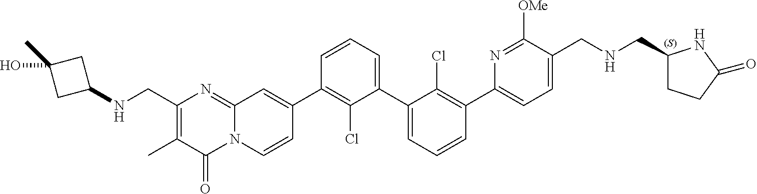

To a solution of tert-butyl ((6-(3′-(2-(((tert-butoxycarbonyl)((1r,3r)-3-hydroxy-3-methylcyclobutyl)amino)methyl)-3-methyl-4-oxo-4H-pyrido[1,2-a]pyrimidin-8-yl)-2,2′-dichloro-[1,1′-biphenyl]-3-yl)-2-methoxypyridin-3-yl)methyl)(((S)-5-oxopyrrolidin-2-yl)methyl)carbamate (250 mg, 0.27 mmol) in DCM (2 mL) was added 2,2,2-trifluoroacetic acid (2.17 mL, 29.4 mmol) and stirred at rt for 0.5 h. The mixture was concentrated in vacuo and the residue was purified by reverse phase HPLC to afford 8-(2,2′-dichloro-3′-(6-methoxy-5-(((((S)-5-oxopyrrolidin-2-yl)methyl)amino)methyl)pyridin-2-yl)-[1,1′-biphenyl]-3-yl)-2-((((1r,3r)-3-hydroxy-3-methylcyclobutyl)amino)methyl)-3-methyl-4H-pyrido[1,2-a]pyrimidin-4-one as a yellow solid (74.8 mg, 35% yield). m/z: 727 [M+H]⁺. ¹H NMR (400 MHz, DMSO-d₆): δ 8.97 (d, J=7.6 Hz, 1H), 8.18 (s, 1H), 7.84 (d, J=7.6 Hz, 1H), 7.72-7.67 (m, 5H), 7.63-7.55 (m, 2H), 7.49-7.44 (m, 2H), 7.29 (d, J=7.2 Hz, 1H), 3.93 (s, 3H), 3.77 (s, 2H), 3.73 (s, 2H), 3.65-3.61 (m, 2H), 2.57 (d, J=6.0 Hz, 2H), 2.20 (s, 3H), 2.16-2.09 (m, 5H), 1.82-1.80 (m, 2H), 1.78-1.69 (m, 1H).

### Example 378: (S)-8-(2,2′-Dichloro-3′-(4-chloro-6-methoxy-5-((((5-oxopyrrolidin-2-yl)methyl)amino) methyl)pyridin-2-yl)-[1,1′-biphenyl]-3-yl)-3-(((2-hydroxyethyl)amino)methyl)-4H-pyrido[1,2-a]pyrimidin-4-one

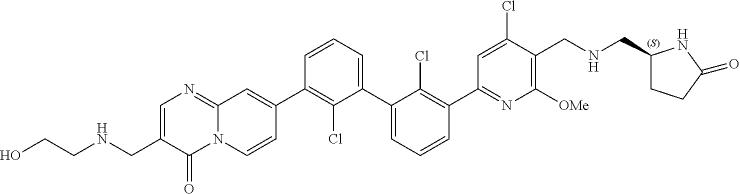

### (S)-5-((((6-(3-Bromo-2-chlorophenyl)-4-chloro-2-methoxypyridin-3-yl)methyl)amino) methyl)pyrrolidin-2-one

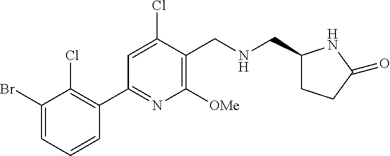 [SMILES: COc1nc(-c2cccc(Br)c2Cl)cc(Cl)c1CNC[C@@H]1CCC(=O)N1]

To a mixture of 6-(3-bromo-2-chloro-phenyl)-4-chloro-2-methoxy-pyridine-3-carbaldehyde (0.5 g, 1.4 mmol) in DCM (20 mL) was added (S)-5-(aminomethyl)pyrrolidin-2-one, HCl (0.31 g, 2.1 mmol) and sodium acetate (0.45 g, 5.5 mmol). The mixture was stirred at rt for 12 h. Sodium cyanoborohydride (0.17 g, 2.8 mmol) was added to the mixture and stirred at rt for 0.5 h. The solvent was evaporated to afford (S)-5-((((6-(3-bromo-2-chlorophenyl)-4-chloro-2-methoxypyridin-3-yl)methyl)amino)methyl)pyrrolidin-2-one. The crude product was used directly without further purification.

### (S)-tert-Butyl ((6-(3-bromo-2-chlorophenyl)-4-chloro-2-methoxypyridin-3-yl)methyl)((5-oxopyrrolidin-2-yl)methyl)carbamate

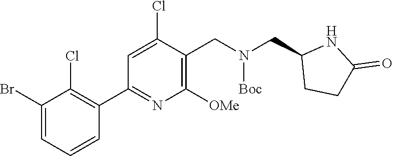 [SMILES: COc1nc(-c2cccc(Br)c2Cl)cc(Cl)c1CN(C[C@@H]1CCC(=O)N1)C(=O)OC(C)(C)C]

To a solution of (S)-5-((((6-(3-bromo-2-chlorophenyl)-4-chloro-2-methoxypyridin-3-yl)methyl)amino)methyl)pyrrolidin-2-one in DCM (20 mL) was added di-tert-butyl dicarbonate (0.96 mL, 4.2 mmol) and triethylamine (0.58 mL, 4.2 mmol). The mixture was stirred at rt for 1.5 h. The mixture was concentrated, and the residue was purified by normal phase SiO₂ chromatography (0-15% EtOAc/petroleum ether) to afford (S)-tert-butyl ((6-(3-bromo-2-chlorophenyl)-4-chloro-2-methoxypyridin-3-yl)methyl)((5-oxopyrrolidin-2-yl)methyl)carbamate as a white solid (0.6 g, 76% yield). ¹H NMR (400 MHz, CDCl₃): δ7.63 (d, J=8 Hz, 1H), 7.42 (d, J=8 Hz, 1H), 7.17-7.15 (m, 2H), 4.66 (br s, 2H), 3.92 (s, 3H), 3.73-3.70 (m, 1H), 3.16-3.15 (m, 2H), 2.24-2.20 (m, 2H), 2.00-1.98 (m, 1H), 1.66-1.62 (m, 1H), 1.56-1.38 (m, 9H).

### (S)-tert-Butyl ((4-chloro-6-(2,2′-dichloro-3′-(3-formyl-4-oxo-4H-pyrido[1,2-a]pyrimidin-8-yl)-[1,1′-biphenyl]-3-yl)-2-methoxypyridin-3-yl)methyl)((5-oxopyrrolidin-2-yl)methyl) carbamate

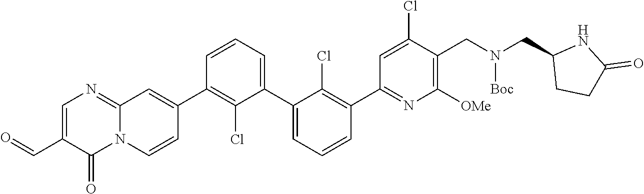 [SMILES: COc1nc(-c2cccc(-c3cccc(-c4ccn5c(=O)c(C=O)cnc5c4)c3Cl)c2Cl)cc(Cl)c1CN(C[C@@H]1CCC(=O)N1)C(=O)OC(C)(C)C]

To a mixture of tert-butyl N-[[6-(3-bromo-2-chloro-phenyl)-4-chloro-2-methoxy-3-pyridyl]methyl]-N-[[(2S)-5-oxopyrrolidin-2-yl]methyl]carbamate (0.4 g, 0.72 mmol), 8-[2-chloro-3-(4,4,5,5-tetramethyl-1,3,2-dioxaborolan-2-yl)phenyl]-4-oxopyrido[1,2-a]pyrimidine-3-carbaldehyde (0.29 g, 0.72 mmol), 2,8-dichloro-5-methoxyquinoline-6-carbaldehyde (47 mg, 72 umol), and potassium phosphate (0.46 g, 2.15 mmol) in dioxane/water mixture (10:1, 44 mL) was stirred at 110° C. for 0.5 h. The reaction mixture was concentrated. The residue was diluted with water (10 mL) and extracted with EtOAc (30 mL). The organic layer was dried over with anhydrous sodium sulfate, filtered and concentrated under reduced pressure. The residue was purified by normal phase SiO₂ chromatography (0-40% EtOAc/petroleum ether) to afford (S)-tert-butyl ((4-chloro-6-(2,2′-dichloro-3′-(3-formyl-4-oxo-4H-pyrido[1,2-a]pyrimidin-8-yl)-[1,1′-biphenyl]-3-yl)-2-methoxypyridin-3-yl)methyl)((5-oxopyrrolidin-2-yl)methyl)carbamate as a yellow solid (0.37 g, 67% yield) ¹H NMR (400 MHz, CDCl₃): δ. 7.63 (d, J=8 Hz, 1H), 7.42 (d, J=7.6 Hz, 1H), 7.17-7.15 (m, 2H), 4.66 (br s, 2H), 3.92 (s, 3H), 3.72-3.70 (m, 1H), 3.17-3.12 (m, 2H), 2.24-2.20 (m, 2H), 1.99-1.96 (m, 1H), 1.66-1.61 (m, 1H), 1.55-1.75 (m, 9H).

### (S)-tert-Butyl ((4-chloro-6-(2,2′-dichloro-3′-(3-(((2-hydroxyethyl)amino)methyl)-4-oxo-4H-pyrido[1,2-a]pyrimidin-8-yl)-[1,1′-biphenyl]-3-yl)-2-methoxypyridin-3-yl)methyl)((5-oxopyrrolidin-2-yl)methyl)carbamate

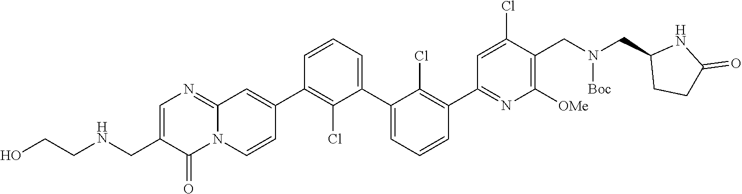 [SMILES: COc1nc(-c2cccc(-c3cccc(-c4ccn5c(=O)c(CNCCO)cnc5c4)c3Cl)c2Cl)cc(Cl)c1CN(C[C@@H]1CCC(=O)N1)C(=O)OC(C)(C)C]

To a mixture of tert-butyl N-[[4-chloro-6-[2-chloro-3-[2-chloro-3-(3-formyl-4-oxo-pyrido[1,2-a]pyrimidin-8-yl)phenyl]phenyl]-2-methoxy-3-pyridyl]methyl]-N-[[(2S)-5-oxopyrrolidin-2-yl]methyl]carbamate (0.2 g, 0.26 mmol) and 2-aminoethanol (48 uL, 0.79 mmol) in DCM (5 mL) was added potassium acetate (65 mg, 0.79 mmol). The mixture was stirred at rt for 12 h. Sodium cyanoborohydride (33 mg, 0.52 mmol) was added and the mixture was stirred at rt for 0.5 h. The mixture was concentrated and the residue was purified by prep-TLC (SiO₂, EtOAc:EtOH:NH₃.H₂O=3:1:0.08) to afford (S)-tert-butyl ((4-chloro-6-(2,2′-dichloro-3′-(3-(((2-hydroxyethyl)amino)methyl)-4-oxo-4H-pyrido[1,2-a]pyrimidin-8-yl)-[1,1′-biphenyl]-3-yl)-2-methoxypyridin-3-yl)methyl)((5-oxopyrrolidin-2-yl)methyl)carbamate as a yellow oil (50 mg, 21% yield).

### (S)-8-(2,2′-Dichloro-3′-(4-chloro-6-methoxy-5-((((5-oxopyrrolidin-2-yl)methyl)amino) methyl)pyridin-2-yl)-[1,1′-biphenyl]-3-yl)-3-(((2-hydroxyethyl)amino)methyl)-4H-pyrido[1,2-a]pyrimidin-4-one

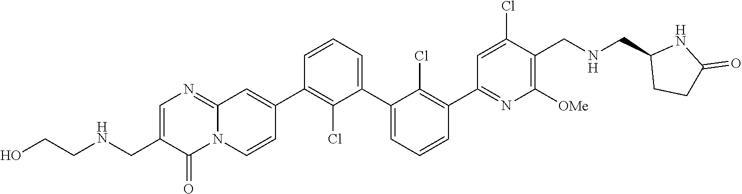 [SMILES: COc1nc(-c2cccc(-c3cccc(-c4ccn5c(=O)c(CNCCO)cnc5c4)c3Cl)c2Cl)cc(Cl)c1CNC[C@@H]1CCC(=O)N1]

To a mixture of (S)-tert-butyl ((4-chloro-6-(2,2′-dichloro-3′-(3-(((2-hydroxyethyl)amino)methyl)-4-oxo-4H-pyrido[1,2-a]pyrimidin-8-yl)-[1,1′-biphenyl]-3-yl)-2-methoxypyridin-3-yl)methyl)((5-oxopyrrolidin-2-yl)methyl)carbamate (50 mg, 62 umol) in DCM (1 mL) was added trifluoroacetic acid (1.5 mL) was stirred at rt for 0.5 h. The mixture was concentrated and the residue was purified by reverse phase HPLC to afford (S)-8-(2,2′-dichloro-3′-(4-chloro-6-methoxy-5-((((5-oxopyrrolidin-2-yl)methyl)amino)methyl)pyridin-2-yl)-[1,1′-biphenyl]-3-yl)-3-(((2-hydroxyethyl)amino)methyl)-4H-pyrido[1,2-a]pyrimidin-4-one (19.8 mg, 40% yield, m/z: 709 [(M+H]⁺ observed). ¹H NMR (400 MHz, DMSO-d₆): δ. 9.12 (d, J=7.6 Hz, 1H), 8.96 (br s, 1H), 8.72 (br s, 3H), 8.57 (s, 1H), 7.91 (d, J=2 Hz, 1H), 7.73-7.57 (m, 9H), 4.35 (s, 2H), 4.22-4.21 (m, 2H), 4.02 (s, 3H), 3.92-3.90 (m, 1H), 3.69-3.87 (m, 2H), 3.22-3.17 (m, 2H), 3.08-3.06 (m, 2H), 2.27-2.15 (m, 3H), 1.82-1.76 (m, 1H).

### Example 393: 8-(2,2′-Dichloro-3′-(4-chloro-6-methoxy-5-(((((S)-5-oxopyrrolidin-2-yl)methyl)amino) methyl)pyridin-2-yl)-[1,1′-biphenyl]-3-yl)-3-((((1r,3r)-3-hydroxy-3-methylcyclobutyl) amino)methyl)-4H-pyrido[1,2-a]pyrimidin-4-one

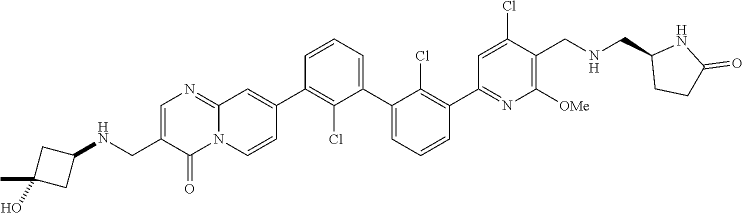

### tert-butyl ((4-Chloro-6-(2,2′-dichloro-3′-(3-((((1r,3r)-3-hydroxy-3-methylcyclobutyl)amino) methyl)-4-oxo-4H-pyrido[1,2-a]pyrimidin-8-yl)-[1,1′-biphenyl]-3-yl)-2-methoxypyridin-3-yl)methyl)(((S)-5-oxopyrrolidin-2-yl)methyl)carbamate

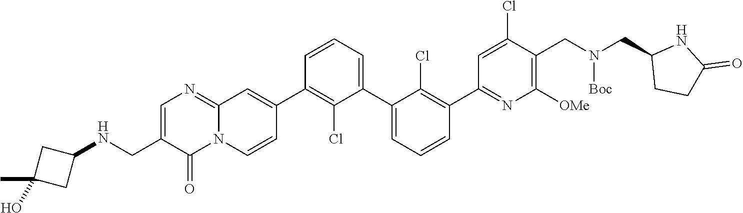

To a mixture of (S)-tert-butyl ((4-chloro-6-(2,2′-dichloro-3′-(3-formyl-4-oxo-4H-pyrido[1,2-a]pyrimidin-8-yl)-[1,1′-biphenyl]-3-yl)-2-methoxypyridin-3-yl)methyl)((5-oxopyrrolidin-2-yl)methyl)carbamate (0.1 g, 0.13 mol) and (1r,3r)-3-amino-1-methylcyclobutanol, HCl (22 mg, 0.16 mmol) in DCM/MeOH mixture (1:1, 4 mL) was added sodium acetate (38 mg, 0.46 mmol). The mixture was stirred at rt for 12 h. Sodium cyanoborohydride (16 mg, 0.26 mmol) was added and the mixture was stirred at rt for 0.5 h. The mixture was concentrated and the residue was purified prep-TLC (SiO₂, EtOAc:EtOH:NH₃.H₂O=3:1:0.04) to afford tert-butyl ((4-chloro-6-(2,2′-dichloro-3′-(3-((((1r,3r)-3-hydroxy-3-methylcyclobutyl)amino)methyl)-4-oxo-4H-pyrido[1,2-a]pyrimidin-8-yl)-[1,1′-biphenyl]-3-yl)-2-methoxypyridin-3-yl)methyl)(((S)-5-oxopyrrolidin-2-yl)methyl)carbamate as a yellow solid (60 mg, 51% yield, m/z: 849 [M+H]⁺ observed).

### 8-(2,2′-Dichloro-3′-(4-chloro-6-methoxy-5-(((((S)-5-oxopyrrolidin-2-yl)methyl)amino) methyl)pyridin-2-yl)-[1,1′-biphenyl]-3-yl)-3-((((1r,3r)-3-hydroxy-3-methylcyclobutyl) amino)methyl)-4H-pyrido[1,2-a]pyrimidin-4-one

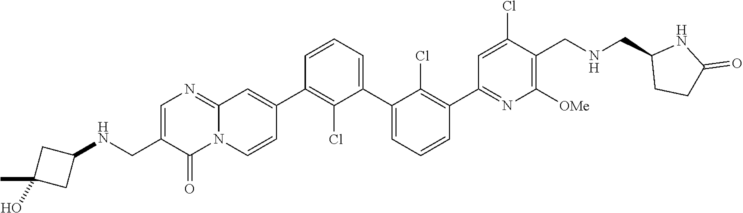

To a mixture of tert-butyl ((4-chloro-6-(2,2′-dichloro-3′-(3-((((1r,3r)-3-hydroxy-3-methylcyclobutyl)amino)methyl)-4-oxo-4H-pyrido[1,2-a]pyrimidin-8-yl)-[1,1′-biphenyl]-3-yl)-2-methoxypyridin-3-yl)methyl)(((S)-5-oxopyrrolidin-2-yl)methyl)carbamate (0.11 mg, 0.13 mmol) in DCM (2 mL) was added triethylamine (3 mL). The mixture was stirred at rt for 0.5 h. The mixture was filtered, and the filtrate was concentrated. The residue was purified by reverse phase HPLC to afford 8-(2,2′-dichloro-3′-(4-chloro-6-methoxy-5-(((((S)-5-oxopyrrolidin-2-yl)methyl)amino)methyl)pyridin-2-yl)-[1,1′-biphenyl]-3-yl)-3-((((1r,3r)-3-hydroxy-3-methylcyclobutyl)amino)methyl)-4H-pyrido[1,2-a]pyrimidin-4-one as a white solid (11.8 mg, 11% yield, m/z: 749 [M+H]⁺ observed). ¹H NMR (400 MHz, DMSO-d₆): δ. 9.03 (d, J=7.6 Hz, 1H), 8.39 (s, 1H), 8.20 (s, 1H), 7.79 (d, J=1.6 Hz, 1H), 7.71-7.64 (m, 6H), 7.59-7.51 (m, 2H), 7.40 (s, 1H), 4.70 (br s, 1H), 3.96 (s, 3H), 3.86 (s, 2H), 3.66 (s, 2H), 3.61-3.59 (m, 1H), 2.55-2.53 (m, 2H), 2.14-2.07 (m, 6H), 1.76-1.67 (m, 4H), 1.25 (s, 3H).

### Example 396: (S)-10-(2,2′-Dichloro-3′-(6-methoxy-5-((((5-oxopyrrolidin-2-yl)methyl)amino)methyl) pyridin-2-yl)-[1,1′-biphenyl]-3-yl)-4,5-dihydro-2H-pyrido[1′,2′:1,2]pyrimido[5,4-f][1,4]oxazepin-6(3H)-one

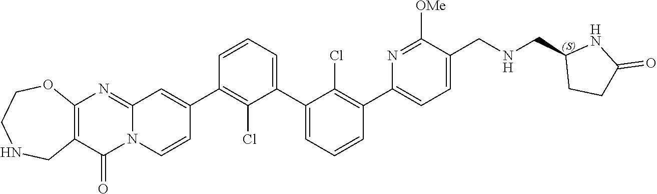

### (S)-tert-Butyl ((6-(2,2′-dichloro-3′-(2-chloro-3-(hydroxymethyl)-4-oxo-4H-pyrido[1,2-a]pyrimidin-8-yl)-[1,1′-biphenyl]-3-yl)-2-methoxypyridin-3-yl)methyl)((5-oxopyrrolidin-2-yl)methyl)carbamate

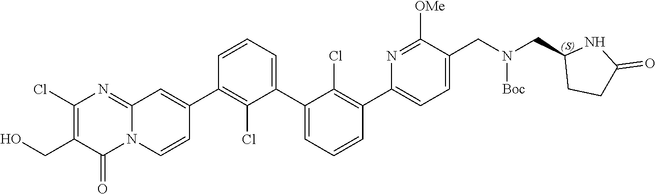

To a mixture of 8-(3-bromo-2-chlorophenyl)-2-chloro-3-(hydroxymethyl)-4H-pyrido[1,2-a]pyrimidin-4-one (1 g, 2.5 mmol) and (S)-tert-butyl ((6-(2-chloro-3-(4,4,5,5-tetramethyl-1,3,2-dioxaborolan-2-yl)phenyl)-2-methoxypyridin-3-yl)methyl)((5-oxopyrrolidin-2-yl)methyl)carbamate (1.72 g, 3 mmol) in dioxane (15 mL) and water (3 mL) was added potassium phosphate (1.6 g, 7.5 mmol) and [1,1′-bis(di-tert-butylphosphino)ferrocene]dichloropalladium(II) (81.5 mg, 0.13 mmol). The mixture was stirred at 110° C. for 0.5 h. The mixture was diluted with water (30 mL) extracted with ethyl acetate (30 mL). The organic phase was dried with anhydrous sodium sulfate, filtered and concentrated. The residue was purified by normal phase SiO₂ chromatography (10-100% in ethyl acetate/petroleum ether) to afford (S)-tert-butyl ((6-(2,2′-dichloro-3′-(2-chloro-3-(hydroxymethyl)-4-oxo-4H-pyrido[1,2-a]pyrimidin-8-yl)-[1,1′-biphenyl]-3-yl)-2-methoxypyridin-3-yl)methyl)((5-oxopyrrolidin-2-yl)methyl)carbamate (1.05 g, 55% yield) as a white solid. ¹H NMR (400 MHz, DMSO-d₆): δ 9.03 (d, J=7.6 Hz, 1H), 7.83 (s, 1H), 7.82 (s, 1H), 7.75-7.60 (m, 6H), 7.58-7.49 (m, 2H), 7.30 (d, J=7.6 Hz, 1H), 4.60-4.58 (m, 2H), 4.06-4.01 (m, 1H), 3.95 (s, 3H), 3.80-3.77 (m, 1H), 2.19-2.06 (m, 4H), 1.78-1.76 (m, 4H), 1.44 (s, 3H), 1.36 (s, 3H), 1.31 (s, 3H).

### (S)-tert-Butyl ((6-(2,2′-dichloro-3′-(2-chloro-3-formyl-4-oxo-4H-pyrido[1,2-a]pyrimidin-8-yl)-[1,1′-biphenyl]-3-yl)-2-methoxypyridin-3-yl)methyl)((5-oxopyrrolidin-2-yl)methyl) carbamate

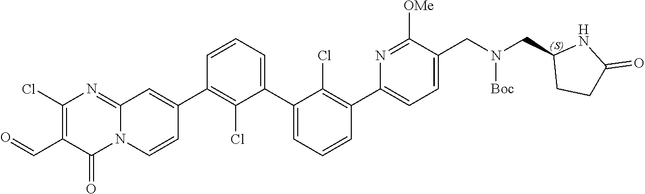

To a mixture of (S)-tert-butyl ((6-(2,2′-dichloro-3′-(2-chloro-3-(hydroxymethyl)-4-oxo-4H-pyrido[1,2-a]pyrimidin-8-yl)-[1,1′-biphenyl]-3-yl)-2-methoxypyridin-3-yl)methyl)((5-oxopyrrolidin-2-yl)methyl)carbamate (1 g, 1.3 mmol) in DCM (20 mL) was added Dess-Martin periodinane (832 mg, 2 mmol). The mixture was stirred at rt for 1.5 h. The reaction mixture was diluted with water (20 mL) and extracted with EtOAc (30 mL). The combined organic layers were concentrated under reduced pressure. The residue was purified by normal phase SiO₂ chromatography (10-100% in ethyl acetate/petroleum ether) to afford (S)-tert-butyl ((6-(2,2′-dichloro-3′-(2-chloro-3-formyl-4-oxo-4H-pyrido[1,2-a]pyrimidin-8-yl)-[1,1′-biphenyl]-3-yl)-2-methoxypyridin-3-yl)methyl)((5-oxopyrrolidin-2-yl)methyl)carbamate (750 mg, 71% yield) as a white solid. ¹H NMR (400 MHz, DMSO-d₆): δ 10.28 (s, 1H), 9.21 (d, J=7.6 Hz, 1H), 8.02 (s, 1H), 7.53 (s, 1H), 7.70-7.67 (m, 3H), 7.58 (s, 2H), 7.51-7.50 (m, 2H), 7.31 (s, 1H), 4.48-4.35 (m, 2H), 3.95 (s, 3H), 3.77 (s, 1H), 3.28 (s, 2H), 2.18-2.06 (m, 4H), 1.78 (s, 1H), 1.44-1.31 (m, 1OH).

### (S)-tert-Butyl ((6-(3′-(2-(2-((tert-butoxycarbonyl)amino)ethoxy)-3-formyl-4-oxo-4H-pyrido[1,2-a]pyrimidin-8-yl)-2,2′-dichloro-[1,1′-biphenyl]-3-yl)-2-methoxypyridin-3-yl)methyl)((5-oxopyrrolidin-2-yl)methyl)carbamate

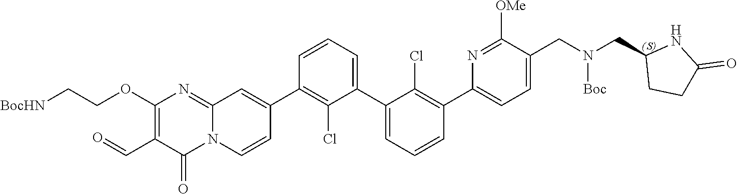

To a mixture of (S)-tert-butyl ((6-(2,2′-dichloro-3′-(2-chloro-3-formyl-4-oxo-4H-pyrido[1,2-a]pyrimidin-8-yl)-[1,1′-biphenyl]-3-yl)-2-methoxypyridin-3-yl)methyl)((5-oxopyrrolidin-2-yl)methyl)carbamate (100 mg, 0.13 mmol) and tert-butyl (2-hydroxyethyl)carbamate (6.45 mmol, 1 mL) in DCM (5 mL) was added triethylamine (66 mg, 0.65 mmol). The mixture was stirred at 50° C. for 12 h. The mixture was diluted with water (10 mL) and extracted with EtOAc (20 mL). The organic phase was dried with anhydrous sodium sulfate, filtered and concentrated. The residue was purified by prep-TLC (33% ethyl acetate/petroleum ether) to afford (S)-tert-butyl (S)-tert-butyl ((6-(3′-(2-(2-((tert-butoxycarbonyl)amino)ethoxy)-3-formyl-4-oxo-4H-pyrido[1,2-a]pyrimidin-8-yl)-2,2′-dichloro-[1,1′-biphenyl]-3-yl)-2-methoxypyridin-3-yl)methyl)((5-oxopyrrolidin-2-yl)methyl)carbamate (70 mg, 30% yield) as a yellow oil. ¹H NMR (400 MHz, CDCl₃): δ 10.33 (s, 1H), 9.10 (d, J=7.6 Hz, 1H), 7.62 (s, 1H), 7.43-7.40 (m, 6H), 7.39-7.36 (m, 1H), 7.22 (s, 1H), 4.55-4.52 (m, 1H), 3.42 (s, 2H), 3.95 (s, 3H), 3.86-3.82 (m, 1H), 3.54-3.49 (m, 2H), 3.30 (s, 2H), 2.28-2.24 (m, 2H), 2.13 (s, 1H), 1.98 (s, 1H), 1.74 (s, 1H), 1.37 (s, 18H), 1.22-1.17 (m, 2H).

### (S)-2-(2-Aminoethoxy)-8-(2,2′-dichloro-3′-(6-methoxy-5-((((5-oxopyrrolidin-2-yl)methyl) amino)methyl)pyridin-2-yl)-[1,1′-biphenyl]-3-yl)-4-oxo-4H-pyrido[1,2-a]pyrimidine-3-carbaldehyde

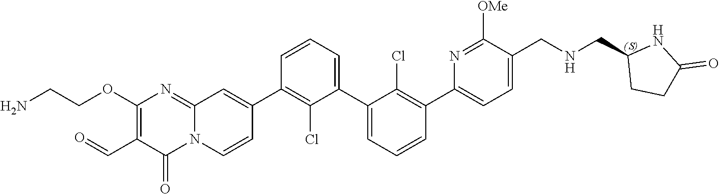

To a mixture of (S)-tert-butyl ((6-(3′-(2-(2-((tert-butoxycarbonyl)amino)ethoxy)-3-formyl-4-oxo-4H-pyrido[1,2-a]pyrimidin-8-yl)-2,2′-dichloro-[1,1′-biphenyl]-3-yl)-2-methoxypyridin-3-yl)methyl)((5-oxopyrrolidin-2-yl)methyl)carbamate (60 mg, 0.07 mmol) in DCM (2 mL) was added trifluoroacetic acid (13.5 mmol, 1 mL). The mixture was stirred at rt for 1 h. The reaction mixture was concentrated under reduced pressure to give crude (S)-2-(2-aminoethoxy)-8-(2,2′-dichloro-3′-(6-methoxy-5-((((5-oxopyrrolidin-2-yl)methyl)amino)methyl)pyridin-2-yl)-[1,1′-biphenyl]-3-yl)-4-oxo-4H-pyrido[1,2-a]pyrimidine-3-carbaldehyde (100 mg, crude, m/z: 687 [M+H]⁺, 689 [M+2]⁺ observed).

### (S)-10-(2,2′-Dichloro-3′-(6-methoxy-5-((((5-oxopyrrolidin-2-yl)methyl)amino)methyl) pyridin-2-yl)-[1,1′-biphenyl]-3-yl)-4,5-dihydro-2H-pyrido[1′,2′:1,2]pyrimido[5,4-f][1,4]oxazepin-6(3H)-one

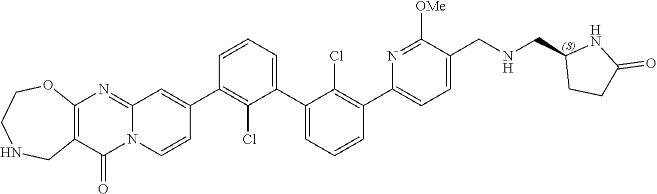

To a mixture of(S)-2-(2-aminoethoxy)-8-(2,2′-dichloro-3′-(6-methoxy-5-((((5-oxopyrrolidin-2-yl)methyl)amino)methyl)pyridin-2-yl)-[1,1′-biphenyl]-3-yl)-4-oxo-4H-pyrido[1,2-a]pyrimidine-3-carbaldehyde (45 mg, 0.06 mmol) in DCM (1 mL) was added triethylamine (99 mg, 0.1 mmol). The mixture was stirred at rt for 0.5 h. Sodium borohydride (7.5 mg, 0.2 mmol) and MeOH (1 mL) was added and the mixture was stirred for 0.5 h. The mixture was concentrated and the residue was purified by reverse phase HPLC to afford (S)-10-(2,2′-dichloro-3′-(6-methoxy-5-((((5-oxopyrrolidin-2-yl)methyl)amino)methyl)pyridin-2-yl)-[1,1′-biphenyl]-3-yl)-4,5-dihydro-2H-pyrido[1′,2′:1,2]pyrimido[5,4-f][1,4]oxazepin-6(3H)-one (2.5 mg, 3% yield, m/z: 671 [M+H]⁺, 673 [M+2]⁺ observed) as a yellow solid. ¹H NMR (400 MHz, DMSO-d₆): δ 8.89 (d, J=7.2 Hz, 1H), 8.20 (s, 1H), 7.81 (d, J=7.6 Hz, 1H), 7.68-7.62 (m, 4H), 7.57-7.55 (m, 3H), 7.47-7.46 (m, 1H), 7.38 (d, J=9.2 Hz, 1H), 7.26 (d, J=7.6 Hz, 1H), 4.34-4.31 (m, 2H), 3.91 (s, 5H), 3.71-3.70 (m, 2H), 3.64-3.62 (m, 2H), 3.09-3.06 (m, 2H), 2.55-2.54 (m, 3H), 2.11-2.08 (m, 2H), 1.74-1.65 (m, 1H).

### Example 374: (S)-10-(2,2′-Dichloro-3′-(6-methoxy-5-((((5-oxopyrrolidin-2-yl)methyl)amino)methyl) pyridin-2-yl)-[1,1′-biphenyl]-3-yl)-1-methyl-2,3,4,5-tetrahydropyrido[1′,2′:1,2]pyrimido [4,5-e][1,4]diazepin-6(1H)-one

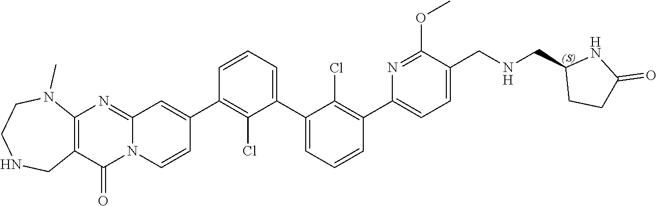

### (S)-tert-Butyl ((6-(3′-(2-((2-((tert-butoxycarbonyl)amino)ethyl)(methyl)amino)-3-formyl-4-oxo-4H-pyrido[1,2-a]pyrimidin-8-yl)-2,2′-dichloro-[1,1′-biphenyl]-3-yl)-2-methoxypyridin-3-yl)methyl)((5-oxopyrrolidin-2-yl)methyl)carbamate

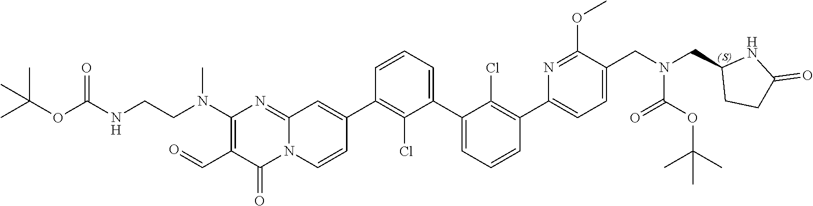

To a mixture of (S)-tert-butyl ((6-(2,2′-dichloro-3′-(2-chloro-3-formyl-4-oxo-4H-pyrido[1,2-a]pyrimidin-8-yl)-[1,1′-biphenyl]-3-yl)-2-methoxypyridin-3-yl)methyl)((5-oxopyrrolidin-2-yl)methyl)carbamate (100 mg, 0.13 mmol) and tert-butyl (2-(methylamino)ethyl)carbamate (20.55 mg, 0.12 mmol) in acetonitrile (3 mL) was added potassium carbonate (18 mg, 0.13 mmol). The mixture was stirred at rt for 12 h. The reaction mixture was diluted with water (5 mL) and extracted with EtOAc (10 mL). The organic phase was dried with anhydrous sodium sulfate, filtered and concentrated. The residue was purified by normal phase SiO₂ chromatography (0-100% in ethyl acetate/petroleum ether) to afford (S)-tert-butyl ((6-(3′-(2-((2-((tert-butoxycarbonyl)amino)ethyl)(methyl)amino)-3-formyl-4-oxo-4H-pyrido[1,2-a]pyrimidin-8-yl)-2,2′-dichloro-[1,1′-biphenyl]-3-yl)-2-methoxypyridin-3-yl)methyl)((5-oxopyrrolidin-2-yl)methyl)carbamate (100 mg, 85% yield, m/z: 900 [M+H]⁺, 902 [M+2]⁺ observed) as a yellow oil.

### (S)-10-(2,2′-Dichloro-3′-(6-methoxy-5-((((5-oxopyrrolidin-2-yl)methyl)amino)methyl) pyridin-2-yl)-[1,1′-biphenyl]-3-yl)-1-methyl-2,3-dihydropyrido[1′,2′:1,2]pyrimido[4,5-e][1,4]diazepin-6(1H)-one

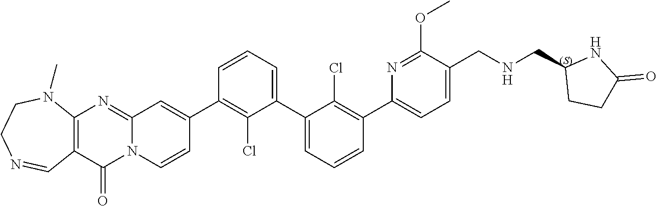

To a mixture of (S)-tert-butyl ((6-(3′-(2-((2-((tert-butoxycarbonyl)amino)ethyl)(methyl)amino)-3-formyl-4-oxo-4H-pyrido[1,2-a]pyrimidin-8-yl)-2,2′-dichloro-[1,1′-biphenyl]-3-yl)-2-methoxypyridin-3-yl)methyl)((5-oxopyrrolidin-2-yl)methyl)carbamate (80 mg, 0.09 mmol) in DCM (2 mL) was added trifluoroacetic acid (13.5 mmol, 1 mL). The mixture was stirred at rt for 1 h. The reaction mixture was concentrated under reduced pressure to give crude (S)-10-(2,2′-dichloro-3′-(6-methoxy-5-((((5-oxopyrrolidin-2-yl)methyl)amino)methyl)pyridin-2-yl)-[1,1′-biphenyl]-3-yl)-1-methyl-2,3-dihydropyrido[1′,2′:1,2]pyrimido[4,5-e][1,4]diazepin-6(1H)-one (100 mg, crude, m/z: 682 [M+H]⁺).

### (S)-10-(2,2′-dichloro-3′-(6-methoxy-5-((((5-oxopyrrolidin-2-yl)methyl)amino)methyl) pyridin-2-yl)-[1,1′-biphenyl]-3-yl)-1-methyl-2,3-dihydropyrido[1′,2′:1,2]pyrimido[4,5-e][1,4]diazepin-6(1H)-one

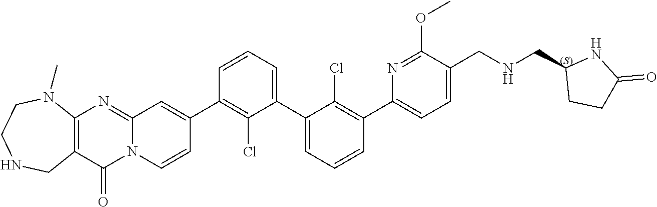

To a mixture of (S)-10-(2,2′-dichloro-3′-(6-methoxy-5-((((5-oxopyrrolidin-2-yl)methyl)amino) methyl)pyridin-2-yl)-[1,1′-biphenyl]-3-yl)-1-methyl-2,3-dihydropyrido[1′,2′:1,2]pyrimido[4,5-e][1,4]diazepin-6(1H)-one (25 mg, 0.04 mmol) in methanol (1 mL) was added sodium borohydride (7 mg, 0.2 mmol). The mixture was stirred at rt for 10 min. The mixture was concentrated and the residue was purified by reverse phase HPLC to afford (S)-10-(2,2′-dichloro-3′-(6-methoxy-5-((((5-oxopyrrolidin-2-yl)methyl)amino)methyl)pyridin-2-yl)-[1,1′-biphenyl]-3-yl)-1-methyl-2,3,4,5-tetrahydropyrido[1′,2′:1,2]pyrimido[4,5-e][1,4]diazepin-6(1H)-one (10.1 mg, 18% yield, m/z: 684 [M+H]⁺). ¹H NMR (400 MHz, DMSO-d₆): δ 8.80 (d, J=7.2 Hz, 1H), 8.26 (s, 1H), 7.83 (d, J=7.6 Hz, 1H), 7.70-7.68 (m, 2H), 7.64-7.63 (m, 2H), 7.62-7.55 (m, 2H), 7.54-7.47 (m, 1H), 7.37 (s, 1H), 7.28 (d, J=7.2 Hz, 1H), 7.17 (d, J=8.8 Hz, 1H), 4.06-4.03 (m, 1H), 4.01-3.93 (m, 4H), 3.65 (s, 2H), 3.60-3.57 (m, 4H), 3.15 (s, 3H), 3.11-3.10 (m, 2H), 2.57 (s, 1H), 2.55-2.51 (m, 1H), 2.11 (s, 2H), 1.99 (s, 1H), 1.73-1.70 (m, 1H), 1.20-1.16 (m, 1H).

### Example 177: 8-(2-Chloro-3-((E)-1-((3-chloro-6-methoxy-5-(((((S)-5-oxopyrrolidin-2-yl)methyl)amino) methyl)pyridin-2-yl)methylene)-2,3-dihydro-1H-inden-4-yl)phenyl)-3-(((((S)-5-oxopyrrolidin-2-yl)methyl)amino)methyl)-4H-pyrido[1,2-a]pyrimidin-4-one

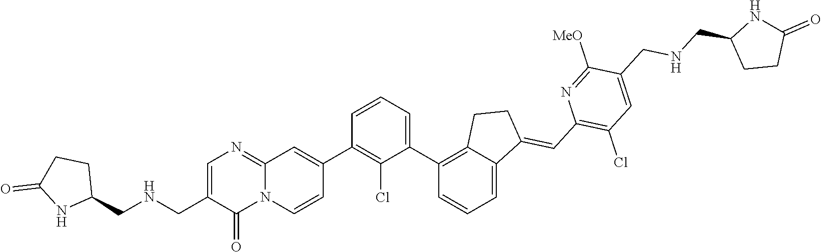 [SMILES: COc1nc(/C=C2\CCc3c2cccc3-c2cccc(-c3ccn4c(=O)c(CNC[C@@H]5CCC(=O)N5)cnc4c3)c2Cl)c(Cl)cc1CNC[C@@H]1CCC(=O)N1]

### 4-Bromo-2,3-dihydro-1H-inden-1-ol

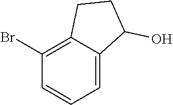 [SMILES: OC1CCc2c(Br)cccc21]

To a mixture of 4-bromo-2,3-dihydro-1H-inden-1-one (5 g, 23.7 mmol) in methanol (50 mL) was added sodium borohydride (2.63 g, 69.6 mmol). The mixture was stirred at rt for 1 h. The reaction mixture was concentrated, and the residue was suspended in water (60 mL). The aqueous layer was extracted with DCM (3×80 mL).The combined organic layers were washed with brine (20 mL), dried over anhydrous sodium sulfate, filtered and concentrated under reduced pressure to give 4-bromo-2,3-dihydro-1H-inden-1-ol as a white solid (6 g, crude). ¹H NMR (400 MHz, DMSO-d₆): δ7.42 (d, J=8 Hz, 1H), 7.33 (d, J=7.6 Hz, 1H), 7.16 (d, J=7.6 Hz, 1H), 5.12 (t, J=6.8 Hz, 1H), 2.92-2.87 (m, 1H), 2.71-2.67 (m, 1H), 2.36-2.33 (m, 1H), 1.82-1.77 (m, 1H).

### 1,4-Dibromo-2,3-dihydro-1H-indene

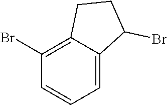 [SMILES: Brc1cccc2c1CCC2Br]

To a mixture of 4-bromo-2,3-dihydro-1H-inden-1-ol (6 g, 28.2 mmol) in DCM (30 mL) was added 45% hydrobromic acid (15 mL, 124 mmol) in water, and then the mixture was stirred at rt for 12 h under N₂ atmosphere. The reaction mixture was diluted with water (50 mL), and then extracted with DCM (3×80 ml). The combined organic layers were washed with brine (50 mL), dried over anhydrous sodium sulfate, filtered and concentrated under reduced pressure to give 1,4-dibromo-2,3-dihydro-1H-indene as yellow oil (5.8 g, crude).

### (4-Bromo-2,3-dihydro-1H-inden-1-yl)triphenylphosphonium bromide

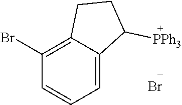

To a mixture of 1,4-dibromo-2,3-dihydro-1H-indene (3 g, 10.9 mmol) in toluene (40 mL) was added triphenylphosphine (3.14 g, 12 mmol) and purged with nitrogen gas. The mixture was stirred at 110° C. for 16 h under nitrogen atmosphere. The reaction mixture was filtered and then the resulting white solid was collected by filtration. The crude product was triturated with PE/EA(3:1, 20 mL) at rt for 0.5 h to get (4-bromo-2,3-dihydro-1H-inden-1-yl) triphenylphosphonium bromide as a white solid (2.8 g, 47% yield, m/z: 457.0/459.0 [M-Br]⁺ observed).

### Methyl 3-chloro-5-formyl-6-hydroxypicolinate

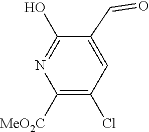 [SMILES: COC(=O)c1nc(O)c(C=O)cc1Cl]

To a mixture of methyl 5-formyl-6-hydroxypicolinate (25 g, 0.14 mol) in DMF (400 mL) was added n-chlorosuccinimide (25.8 g, 0.19 mol) and 1.5 mol of H₂SO₄ (80 mL). The mixture was stirred at 80° C. for 12 h under nitrogen. The mixture was diluted with water (150 mL) and extracted with DCM (3×400 mL). The combined organic phase was dried with anhydrous sodium sulfate, filtered and concentrated. The residue was purified by normal phase SiO₂ chromatography (0-90% EtOAc/petroleum ether) to afford methyl 3-chloro-5-formyl-6-hydroxypicolinate as a yellow solid (25 g, 35% yield, m/z: 216.0 [M+H]⁺ observed).

### Methyl 3-chloro-5-(1,3-dioxolan-2-yl)-6-hydroxypicolinate

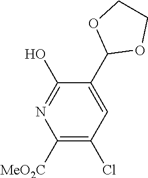 [SMILES: COC(=O)c1nc(O)c(C2OCCO2)cc1Cl]

A mixture of methyl 3-chloro-5-formyl-6-hydroxypicolinate (25 g, 116 mmol), ethyleneglycol (28.8 g, 463 mmo), p-Toluenesulfonic acid (2 g, 11.6 mmol) in toluene (400 mL) was degassed and purged with nitrogen. The mixture was stirred at 130° C. for 1 h under nitrogen atmosphere. The reaction was quenched with sodium carbonate (10 g) in water (150 mL). The aqueous layer was extracted with DCM (2×400 mL). The combined organic phase was dried with anhydrous sodium sulfate, filtered and concentrated. The residue was purified by normal phase SiO₂ chromatography (0-100% EtOAc/petroleum ether) to afford methyl 3-chloro-5-(1,3-dioxolan-2-yl)-6-hydroxypicolinate as a yellow solid (15 g, 41% yield).

### Methyl 3-chloro-5-(1,3-dioxolan-2-yl)-6-methoxypicolinate

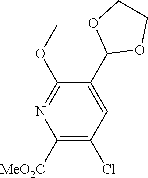 [SMILES: COC(=O)c1nc(OC)c(C2OCCO2)cc1Cl]

To a mixture of methyl 3-chloro-5-(1,3-dioxolan-2-yl)-6-hydroxypicolinate (14 g, 53.9 mmol) in CHCl₃ (250 mL) was added Mel (45.9 g, 323 mmol) and silver carbonate (29.7 g, 107 mmol). The mixture was stirred rt for 12 h under nitrogen. The reaction was quenched with ammonium chloride (20 g) in water (200 mL). The mixture was filtered and the filtrate was extracted with ethyl acetate (3×200 mL). The combined organic phase was dried with anhydrous sodium sulfate, filtered and concentrated. The residue was purified by normal phase SiO₂ chromatography (0-40% EtOAc/petroleum ether) to afford methyl 3-chloro-5-(1,3-dioxolan-2-yl)-6-methoxypicolinate as a yellow solid (7 g). ¹H NMR (400 MHz, CDCl₃): δ 7.84 (s, 1H), 5.99 (s, 1H), 4.12-4.04 (m, 4H), 4.01 (s, 3H), 3.97 (s, 3H).

### (3-Chloro-5-(1,3-dioxolan-2-yl)-6-methoxypyridin-2-yl)methanol

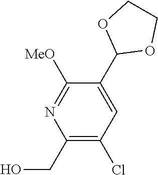 [SMILES: COc1nc(CO)c(Cl)cc1C1OCCO1]

To a mixture of methyl 3-chloro-5-(1,3-dioxolan-2-yl)-6-methoxypicolinate (6.5 g, 23.8 mmol) in DCM (52 mL) was added diisobutylaluminumhydride (1 M, 59.3 mL) at 0° C. The mixture was stirred at 0° C. for 1 h. The reaction was quenched carefully with saturated ammonium chloride (50 mL). The reaction mixture was diluted with water (30 mL) and extracted with THF/EtOAc (3/1, 3×80 mL). The residue was purified by normal phase SiO₂ chromatography (0-100% EtOAc/petroleum ether) to afford (3-chloro-5-(1,3-dioxolan-2-yl)-6-methoxypyridin-2-yl)methanol as a white solid. ¹H NMR (400 MHz, DMSO-d₆): δ 7.76 (s, 1H), 5.90 (s, 1H), 5.16 (t, J=6 Hz, 1H), 4.56 (d, J=5.6 Hz, 2H), 4.07-4.06 (m, 2H), 3.96-3.92 (m, 5H).

### 3-Chloro-5-(1,3-dioxolan-2-yl)-6-methoxypicolinaldehyde

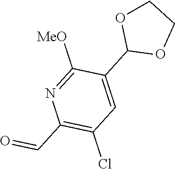 [SMILES: COc1nc(C=O)c(Cl)cc1C1OCCO1]

To a mixture of (3-chloro-5-(1,3-dioxolan-2-yl)-6-methoxypyridin-2-yl)methanol (2 g, 8.14 mmol) in CHCl₃ (20 mL) was added Dess-Martin (3.45 g, 8.14 mmol). The mixture was stirred at 15° C. for 4 h. The reaction mixture was concentrated under reduced pressure to give a residue. The residue was purified by normal phase SiO₂ chromatography (0-100% EtOAc/petroleum ether) to afford 3-chloro-5-(1,3-dioxolan-2-yl)-6-methoxypicolinaldehyde as a yellow solid (1.5 g, 75% yield). ¹H NMR (400 MHz, DMSO-d₆): δ 10.14 (s, 1H), 8.01 (s, 1H), 6.02 (s, 1H), 4.22-4.01 (m, 7H).

### (E)-2-((4-Bromo-2,3-dihydro-1H-inden-1-ylidene)methyl)-3-chloro-5-(1,3-dioxolan-2-yl)-6-methoxypyridine

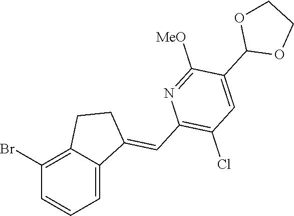 [SMILES: COc1nc(/C=C2\CCc3c(Br)cccc32)c(Cl)cc1C1OCCO1]

To a mixture of (4-bromo-2,3-dihydro-1H-inden-1-yl)triphenylphosphonium bromide (2.14 g, 4.1 mmol) in DCM (20 mL) was added 3-chloro-5-(1,3-dioxolan-2-yl)-6-methoxy-picolinaldehyde (1 g, 4.1 mmol), potassium carbonate (5.67 g, 41 mmol) and 18-CROWN-6 (10.84 mg, 0.04 mmol). The mixture was stirred at 40° C. for 4 h. The reaction was quenched with water (30 mL), and then extracted with EtOAc (3×60 ml). The combined organic layers were washed with brine (20 mL), dried over anhydrous sodium sulfate, filtered and concentrated. The residue was purified by normal phase SiO₂ chromatography (0-100% EtOAc/petroleum ether) to afford to get the crude product. The crude product was triturated with EtOH (20 mL) at 25° C. for 30 min to get (E)-2-((4-bromo-2,3-dihydro-1H-inden-1-ylidene)methyl)-3-chloro-5-(1,3-dioxolan-2-yl)-6-methoxypyridine (1 g, 57% yield). ¹H NMR (400 MHz, DMSO-d₆): δ 7.82-7.79 (m, 2H), 7.57 (d, J=7.6 Hz, 1H), 7.33 (t, J=2.4 Hz, 1H), 7.27 (t, J=7.6 Hz, 1H), 5.91 (s, 1H), 4.09-3.94 (m, 7H), 3.49-3.33 (m, 2H), 3.06-3.03 (m, 2H).

### (E)-8-(2-Chloro-3-(1-((3-chloro-5-(1,3-dioxolan-2-yl)-6-methoxypyridin-2-yl)methylene)-2,3-dihydro-1H-inden-4-yl)phenyl)-4-oxo-4H-pyrido[1,2-a]pyrimidine-3-carbaldehyde

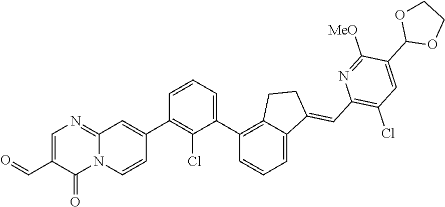 [SMILES: COc1nc(/C=C2\CCc3c2cccc3-c2cccc(-c3ccn4c(=O)c(C=O)cnc4c3)c2Cl)c(Cl)cc1C1OCCO1]

To a mixture of 8-(2-chloro-3-(4,4,5,5-tetramethyl-1,3,2-dioxaborolan-2-yl)phenyl)-4-oxo-4H-pyrido[1,2-a]pyrimidine-3-carbaldehyde (0.73 g, 1.77 mmol) in dioxane/Water/THE (2:2:1, 5 mL) was added (E)-2-((4-bromo-2,3-dihydro-1H-inden-1-ylidene)methyl)-3-chloro-5-(1,3-dioxolan-2-yl)-6-methoxypyridine (0.5 g, 1.18 mmol), potassium phosphate (0.75 g, 3.55 mmol) and [1,1′-Bis(di-tert-butylphosphino)ferrocene]dichloropalladium(II) (0.08 g, 0.12 mmol). The mixture was stirred at 80° C. for 2 h. The reaction mixture was diluted with water (30 mL) and extracted with EtOAc (3×30 ml).The combined organic layers were washed with brine (20 mL), dried over anhydrous sodium sulfate, filtered and concentrated. The residue was purified by normal phase SiO₂ chromatography (0-100% EtOAc/petroleum ether) to afford (E)-8-(2-chloro-3-(1-((3-chloro-5-(1,3-dioxolan-2-yl)-6-methoxypyridin-2-yl)methylene)-2,3-dihydro-1H-inden-4-yl)phenyl)-4-oxo-4H-pyrido[1,2-a]pyrimidine-3-carbaldehyde as a yellow solid (0.4 g, 54% yield).

### (E)-8-(2-Chloro-3-(1-((3-chloro-5-formyl-6-methoxypyridin-2-yl)methylene)-2,3-dihydro-1H-inden-4-yl)phenyl)-4-oxo-4H-pyrido[1,2-a]pyrimidine-3-carbaldehyde

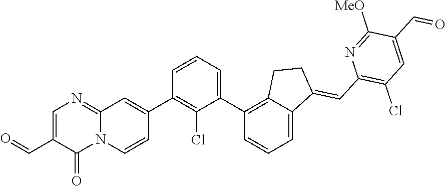 [SMILES: COc1nc(/C=C2\CCc3c2cccc3-c2cccc(-c3ccn4c(=O)c(C=O)cnc4c3)c2Cl)c(Cl)cc1C=O]

To a mixture of(E)-8-(2-chloro-3-(1-((3-chloro-5-(1,3-dioxolan-2-yl)-6-methoxypyridin-2-yl)methylene)-2,3-dihydro-1H-inden-4-yl)phenyl)-4-oxo-4H-pyrido[1,2-a]pyrimidine-3-carbaldehyde (0.15 g, 0.24 mmol) in THF (12 mL) was added concentrated HCl (6.00 mL). The mixture was stirred at rt for 1 h. The solvent was evaporated, and the residue poured into ice-water mixture. Th mixture was neutralized with saturated aqueous sodium bicarbonate solution, and the aqueous layer was extracted with EtOAc (3×30 ml). The combined organic layers were washed with brine (20 mL), dried over anhydrous sodium sulfate, filtered and concentrated under reduced pressure to give (E)-8-(2-chloro-3-(1-((3-chloro-5-formyl-6-methoxypyridin-2-yl)methylene)-2,3-dihydro-1H-inden-4-yl)phenyl)-4-oxo-4H-pyrido[1,2-a]pyrimidine-3-carbaldehyde as a yellow solid (0.15 g, crude).

### 8-(2-Chloro-3-((E)-1-((3-chloro-6-methoxy-5-(((((S)-5-oxopyrrolidin-2-yl)methyl)amino) methyl)pyridin-2-yl)methylene)-2,3-dihydro-1H-inden-4-yl)phenyl)-3-(((((S)-5-oxopyrrolidin-2-yl)methyl)amino)methyl)-4H-pyrido[1,2-a]pyrimidin-4-one

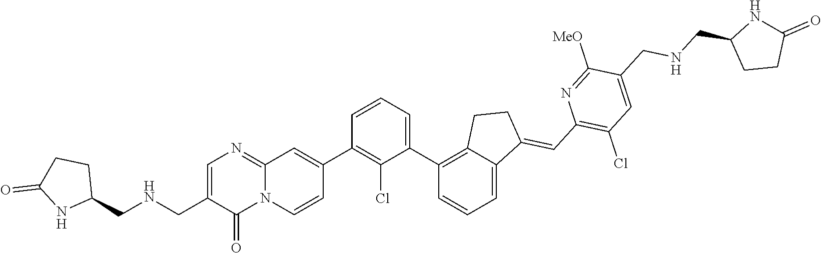 [SMILES: COc1nc(/C=C2\CCc3c2cccc3-c2cccc(-c3ccn4c(=O)c(CNC[C@@H]5CCC(=O)N5)cnc4c3)c2Cl)c(Cl)cc1CNC[C@@H]1CCC(=O)N1]

To a mixture of (E)-8-(2-chloro-3-(1-((3-chloro-5-formyl-6-methoxypyridin-2-yl)methylene)-2,3-dihydro-1H-inden-4-yl)phenyl)-4-oxo-4H-pyrido[1,2-a]pyrimidine-3-carbaldehyde (0.15 g, 0.24 mmol) in MeOH/DCM (1:1, 6 mL) was added ((S)-5-(aminomethyl)pyrrolidin-2-one, HCl (0.22 g, 1.45 mmol) and sodium acetate (0.12 g, 1.45 mmol). The mixture was stirred at rt for 2 h. Sodium cyanoborohydride (0.1 g, 1.45 mmol) was added and the mixture was stirred at rt for 1 hour. The reaction mixture was filtered, and the filtrate was concentrated under reduced pressure to give a residue. The residue was purified by reverse phase HPLC to afford 8-(2-chloro-3-((E)-1-((3-chloro-6-methoxy-5-(((((S)-5-oxopyrrolidin-2-yl)methyl)amino)methyl)pyridin-2-yl)methylene)-2,3-dihydro-1H-inden-4-yl)phenyl)-3-(((((S)-5-oxopyrrolidin-2-yl)methyl) amino)methyl)-4H-pyrido[1,2-a]pyrimidin-4-one as a yellow solid (28.6 mg, 14% yield, m/z: 780.2 [M+H]⁺ observed). ¹H NMR (400 MHz, DMSO-d₆): δ 9.10 (d, J=8 Hz, 1H), 9.40 (s, 1H), 7.83 (d, J=8 Hz, 1H), 7.79-7.69 (m, 2H), 7.65-7.58 (m, 4H), 7.55-7.49 (m, 2H), 7.43 (t, J=7.6 Hz, 1H), 7.36 (m, 1H), 7.28 (d, J=7.6 Hz, 1H), 3.95 (s, 3H), 3.72 (s, 2H), 3.65-3.61 (m, 4H), 3.44 (m, 2H), 2.91-2.89 (m, 2H), 2.53-2.52 (m, 4H), 2.25 (m, 1H), 2.16-2.02 (m, 6H), 1.70-1.67 (m, 2H).

### Example 375: (S)-5-((((6-(2,2′-Dichloro-3′-(1-methyl-3-(((((S)-5-oxopyrrolidin-2-yl)methyl)amino)methyl)-1H-pyrrolo[3,2-b]pyridin-6-yl)-[1,1′-biphenyl]-3-yl)-2-methoxypyridin-3-yl)methyl)amino) methyl)pyrrolidin-2-one

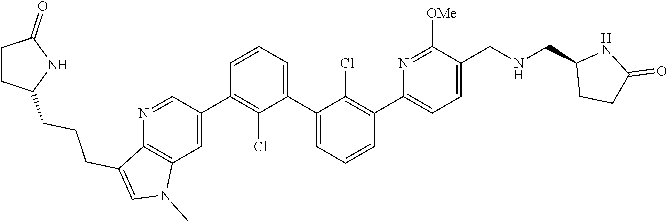

### 6-Bromo-1-methyl-1H-pyrrolo[3,2-b]pyridine-3-carbaldehyde

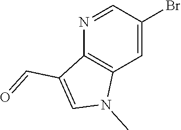

To a mixture of 6-bromo-1H-pyrrolo[3,2-b]pyridine-3-carbaldehyde (400 mg, 1.78 mmol) in DMF (8 mL) was added sodium hydride (107 mg, 2.67 mmol, 60% purity) at 0° C. followed by chloroiodomethane (303 mg, 2.13 mmol). The mixture was stirred at rt for 1.5 h under N₂. The reaction was quenched with water (20 mL). The aqueous layer was extracted with EtOAc (50 mL×3). The combined organic phase was dried over anhydrous sodium sulfate, filtered and concentrated under reduced pressure to afford 6-bromo-1-methyl-1H-pyrrolo[3,2-b]pyridine-3-carbaldehyde (300 mg, 70% yield) as a brown solid. (1H NMR) The product was used directly without further purification. ¹H NMR (400 MHz, DMSO-d₆): δ 10.12 (s, 1H), 8.60 (d, J=2 Hz, 1H), 8.48 (s, 1H), 8.43 (d, J=2 Hz, 1H), 3.92 (s, 3H).

### 6-(2,2′-Dichloro-3′-(5-formyl-6-methoxypyridin-2-yl)-[1,1′-biphenyl]-3-yl)-1-methyl-1H-pyrrolo[3,2-b]pyridine-3-carbaldehyde

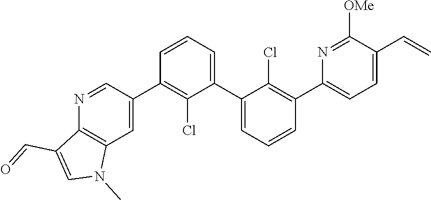 [SMILES: C=Cc1ccc(-c2cccc(-c3cccc(-c4cnc5c(C=O)cn(C)c5c4)c3Cl)c2Cl)nc1OC]

To a mixture of 6-bromo-1-methyl-1H-pyrrolo[3,2-b]pyridine-3-carbaldehyde (150 mg, 0.63 mmol), 6-(2,2′-dichloro-3′-(4,4,5,5-tetramethyl-1,3,2-dioxaborolan-2-yl)-[1,1′-biphenyl]-3-yl)-2-methoxynicotinaldehyde (456 mg, 0.94 mmol) in dioxane/water mixture (5:1, 12.5 mL) was added potassium carbonate (260 mg, 1.88 mmol), [1,1-bis(diphenylphosphino)ferrocene]palladium(II) chloride dichloromethane complex (51.2 mg, 0.06 mmol). The mixture was stirred at 110° C. for 0.5 h under N₂. The reaction mixture was concentrated, and the residue was purified by normal phase SiO₂ chromatography (0-80% EtOAc/petroleum ether) to afford 6-(2,2′-dichloro-3′-(5-formyl-6-methoxypyridin-2-yl)-[1,1′-biphenyl]-3-yl)-1-methyl-1H-pyrrolo[3,2-b]pyridine-3-carbaldehyde (220 mg, 68% yield) as a yellow solid. ¹H NMR (400 MHz, DMSO-d₆): δ 10.27 (s, 1H), 10.16 (s, 1H), 8.60 (d, J=1.6 Hz, 1H), 8.49 (s, 1H), 8.20-8.18 (m, 2H), 7.72 (dd, J₁=1.6 Hz, J₂=7.6 Hz, 1H), 7.61-7.46 (m, 6H), 4.04 (s, 3H), 3.94 (s, 3H).

### (S)-5-((((6-(2,2′-Dichloro-3′-(1-methyl-3-(((((S)-5-oxopyrrolidin-2-yl)methyl)amino) methyl)-1H-pyrrolo[3,2-b]pyridin-6-yl)-[1,1′-biphenyl]-3-yl)-2-methoxypyridin-3-yl)methyl)amino)methyl)pyrrolidin-2-one

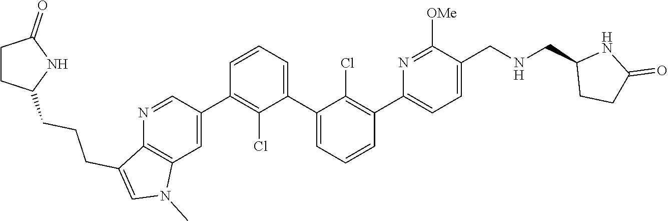

To a mixture of 6-(2,2′-dichloro-3′-(5-formyl-6-methoxypyridin-2-yl)-[1,1′-biphenyl]-3-yl)-1-methyl-1H-pyrrolo[3,2-b]pyridine-3-carbaldehyde (200 mg, 0.39 mmol), (5S)-5-(aminomethyl) pyrrolidin-2-one, HCl (350 mg, 2.32 mmol) in DCM (10 mL) was added sodium acetate (191 mg, 2.32 mmol). The mixture was stirred at rt for 2 h under N₂. Sodium cyanoborohydride (73 mg, 1.16 mmol) was added to the mixture, and the mixture was stirred at rt for 0.5 h. The mixture was concentrated and the residue was purified by reverse phase HPLC to afford (S)-5-((((6-(2,2′-dichloro-3′-(1-methyl-3-(((((S)-5-oxopyrrolidin-2-yl)methyl)amino)methyl)-1H-pyrrolo[3,2-b]pyridin-6-yl)-[1,1′-biphenyl]-3-yl)-2-methoxypyridin-3-yl)methyl)amino) methyl)pyrrolidin-2-one (40.2 mg, m/z: 712 [M+H]⁺ observed) as a white solid. ¹H NMR (400 MHz, DMSO-d₆): δ 8.45 (d, J=1.6 Hz, 1H), 8.01 (d, J=1.6 Hz, 1H), 7.83 (d, J=7.2 Hz, 1H), 7.71-7.66 (m, 3H), 7.59-7.55 (m, 3H), 7.48-7.45 (m, 2H), 7.28 (d, J=7.6 Hz, 1H), 4.11 (s, 2H), 4.07 (s, 3H), 4.03 (s, 3H), 3.77-3.65 (m, 2H), 2.77-2.72 (m, 2H), 2.57 (d, J=6 Hz, 2H), 2.18-2.05 (m, 6H), 1.75-1.67 (m, 2H).

### Example 387: 2-(2,2′-Dichloro-3′-(6-methoxy-5-(((((S)-5-oxopyrrolidin-2-yl)methyl)amino)methyl)pyridin-2-yl)-[1,1′-biphenyl]-3-yl)-5-(((((S)-5-oxopyrrolidin-2-yl)methyl)amino)methyl)isoindolin-1-one

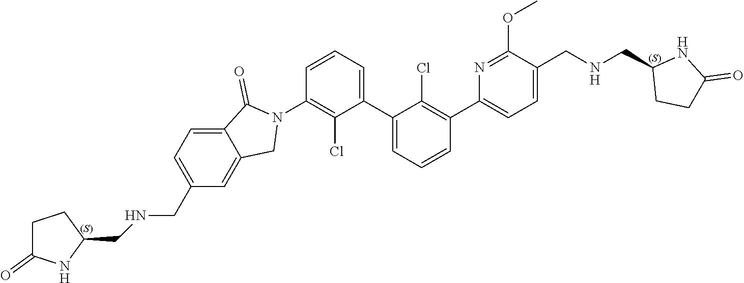

### 2-(2,2′-Dichloro-3′-(5-formyl-6-methoxypyridin-2-yl)-[1,1′-biphenyl]-3-yl)-1-oxoisoindoline-5-carbaldehyde

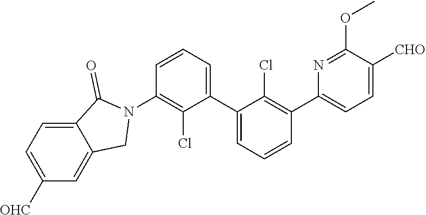 [SMILES: COc1nc(-c2cccc(-c3cccc(N4Cc5cc(C=O)ccc5C4=O)c3Cl)c2Cl)ccc1C=O]

To a mixture 1-oxoisoindoline-5-carbaldehyde (0.14 g, 0.77 mmol) in tert-amyl alcohol (14 mL) was added 6-(3′-bromo-2,2′-dichloro-[1,1′-biphenyl]-3-yl)-2-methoxynicotinaldehyde (0.35 g, 0.77 mmol), [2-(2-aminophenyl)phenyl]-methylsulfonyloxy-palladium;dicyclohexyl-[3,6-dimethoxy-2-(2,4,6-triisopropylphenyl)phenyl]phosphane (0.07 g, 0.07 mmol) and cesium carbonate (0.49 g, 1.61 mmol). The mixture was stirred at 100° C. for 12 h. The reaction mixture was filtered, and the filtrate was concentrated. The filtrate was diluted with water (30 mL) and extracted with EtOAc (3×30 ml). The combined organic layers were washed with brine (20 mL), dried over anhydrous sodium sulfate, filtered and concentrated. The residue was purified by reverse phase HPLC to afford 2-(2,2′-dichloro-3′-(5-formyl-6-methoxypyridin-2-yl)-[1,1′-biphenyl]-3-yl)-1-oxoisoindoline-5-carbaldehyde as a yellow solid (75 mg, m/z:517.1[M+H]⁺ observed).

### 2-(2,2′-Dichloro-3′-(6-methoxy-5-(((((S)-5-oxopyrrolidin-2-yl)methyl)amino)methyl)pyridin-2-yl)-[1,1′-biphenyl]-3-yl)-5-(((((S)-5-oxopyrrolidin-2-yl)methyl)amino)methyl)isoindolin-1-one

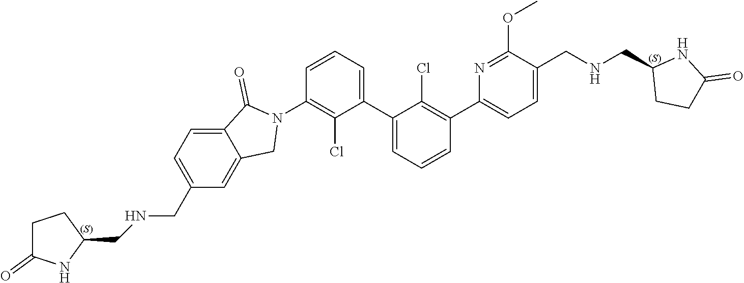

To a mixture of 2-(2,2′-dichloro-3′-(5-formyl-6-methoxypyridin-2-yl)-[1,1′-biphenyl]-3-yl)-1-oxoisoindoline-5-carbaldehyde (0.08 g, 0.05 mmol) in MeOH (2 mL) was added (S)-5-(aminomethyl)pyrrolidin-2-one, HCl (0.02 g, 0.14 mmol) and sodium acetate (0.02 g, 0.19 mmol). The mixture was stirred at 25° C. for 1 h. Sodium cyanoborohydride (0.009 g, 0.14 mmol) was added and the mixture was stirred at 25° C. for 0.5 h. The reaction mixture was filtered and concentrated under reduced pressure. The residue was purified by reverse phase HPLC to afford 2-(2,2′-dichloro-3′-(6-methoxy-5-(((((S)-5-oxopyrrolidin-2-yl)methyl)amino) methyl)pyridin-2-yl)-[1,1′-biphenyl]-3-yl)-5-(((((S)-5-oxopyrrolidin-2-yl)methyl)amino)methyl) isoindolin-1-one as a yellow solid (8.0 mg, 22% yield, m/z:713.3[M+H]⁺ observed). ¹H NMR (400 MHz, Methanol-d₄) δ 7.90 (d, J=7.6 Hz, 1H), 7.84 (d, J=7.6 Hz, 1H), 7.72 (s, 1H), 7.68-7.51 (m, 5H), 7.50-7.43 (m, 2H), 7.34-7.32 (d, J=7.6 Hz, 1H), 4.96 (s, 1H), 4.16-4.08 (m, 7H), 3.99-3.80 (m, 2H), 3.06-3.00 (m, 2H), 2.91-2.80 (m, 2H), 2.42-2.29 (m, 6H), 1.92-1.84 (m, 2H); ¹H NMR (400 MHz, DMSO-d₆) δ7.84 (d, J=7.6 Hz, 1H), 7.75 (d, J=8 Hz, 1H), 7.68-7.65 (m, 5H), 7.60-7.54 (m, 3H), 7.50-7.43 (m, 2H), 7.28 (d, J=7.2 Hz, 1H), 4.88 (s, 2H), 3.93 (s, 3H), 3.87 (d, J=3.2 Hz, 2H), 3.74 (m, 2H), 3.64 (m, 3H), 2.58-2.57 (m, 3H), 2.18-2.08 (m, 7H), 1.76-1.65 (m, 3H).

### Example 394: (5S,5'S)-5,5′-((6,6′-(2,2′-Dichloro-[1,1′-biphenyl]-3,3′-diyl)bis(8-methoxy-3,4-dihydroisoquinoline-6,2(1H)-diyl))bis(methylene))bis(pyrrolidin-2-one)

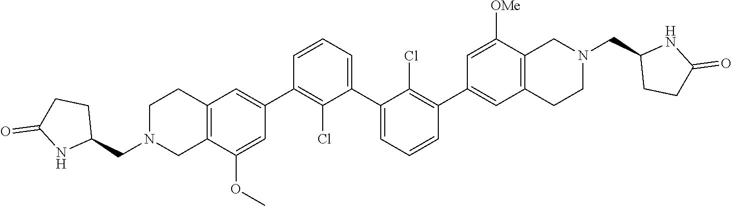 [SMILES: COc1cc(-c2cccc(-c3cccc(-c4cc5c(c(OC)c4)CN(C[C@@H]4CCC(=O)N4)CC5)c3Cl)c2Cl)cc2c1CN(C[C@@H]1CCC(=O)N1)CC2]

### (S)-5-((6-Bromo-8-methoxy-3,4-dihydroisoquinolin-2(1H)-yl)methyl)pyrrolidin-2-one

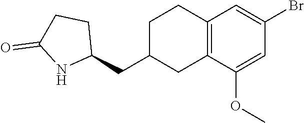 [SMILES: COc1cc(Br)cc2c1CC(C[C@@H]1CCC(=O)N1)CC2]

To a mixture of 6-bromo-8-methoxy-1,2,3,4-tetrahydroisoquinoline, HCl (400 mg, 1.65 mmol) in acetonitrile (100 mL) was added (S)-5-(bromomethyl)pyrrolidin-2-one (540 mg, 3.03 mmol) and potassium carbonate (685 mg, 4.96 mmol). The reaction was stirred for 12 h at 100° C. The reaction was quenched with water (100 mL) and extracted with EtOAc (200 mL). The organic layer was separated, dried over sodium sulfate, filtered and concentrated under vacuum. The residue was purified by normal phase SiO₂ chromatography (0-100% EtOAc/petroleum ether to 0-100% THF/EtOAc) to afford (S)-5-((6-bromo-8-methoxy-3,4-dihydroisoquinolin-2(1H)-yl)methyl)pyrrolidin-2-one as a yellow solid (0.76 g).

### (5S,5'S)-5,5′-((6,6′-(2,2′-Dichloro-[1,1′-biphenyl]-3,3′-diyl)bis(8-methoxy-3,4-dihydroisoquinoline-6,2(1H)-diyl))bis(methylene))bis(pyrrolidin-2-one)

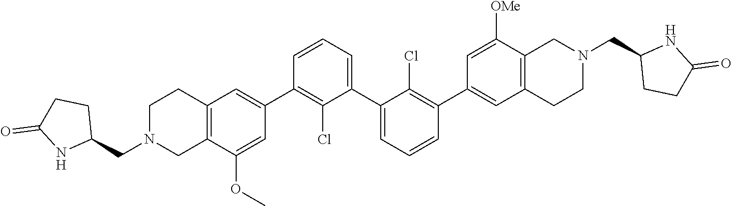 [SMILES: COc1cc(-c2cccc(-c3cccc(-c4cc5c(c(OC)c4)CN(C[C@@H]4CCC(=O)N4)CC5)c3Cl)c2Cl)cc2c1CN(C[C@@H]1CCC(=O)N1)CC2]

To a mixture of (S)-5-((6-bromo-8-methoxy-3,4-dihydroisoquinolin-2(1H)-yl)methyl) pyrrolidin-2-one (214 mg, 0.63 mmol) and 2,2′-(2,2′-dichloro-[1,1′-biphenyl]-3,3′-diyl) bis(4,4,5,5-tetramethyl-1,3,2-dioxaborolane) (100 mg, 0.21 mmol) in dioxane/water (10/1, 1.1 mL) was added 1,1′-bis(diphenylphosphino)ferrocene-palladium(ii) dichloride dichloromethane complex (8.60 mg, 0.01 mmol) and potassium carbonate (87.3 mg, 0.63 mmol). The reaction mixture was stirred for 0.5 h at 110° C. After cooling, the mixture was diluted with water (20 mL) and extracted with EtOAc (200 mL). The organic layer was separated, dried over sodium sulfate, filtered and concentrated under vacuum. The residue was purified by normal phase SiO₂ chromatography (0-100% EtOAc/petroleum ether to 0-100% MeOH/EtOAc) followed by reverse phase HPLC to afford (5S,5'S)-5,5′-((6,6′-(2,2′-dichloro-[1,1′-biphenyl]-3,3′-diyl) bis(8-methoxy-3,4-dihydroisoquinoline-6,2(1H)-diyl))bis(methylene))bis(pyrrolidin-2-one) as a white solid (10.2 mg, 6% yield). m/z: 739 [M+H]⁺. ¹H NMR (400 MHz, DMSO-d₆): δ 7.61 (s, 2H), 7.51-7.44 (m, 4H), 7.37 (dd, J=7.2 Hz, J=2.0 Hz, 2H), 6.81 (d, J=7.6 Hz, 3H), 3.80 (s, 3H), 3.79-3.77 (m, 1H), 3.60-3.47 (m, 4H), 2.84-2.83 (m, 4H), 2.71-2.67 (m, 4H), 2.57-2.2.54 (m, 3H), 2.48-2.46 (m, 2 H), 2.22-2.09 (m, 6H), 1.76-1.70 (m, 2H).

### Example 400: 6-(2,2′-Dichloro-3′-(6-methoxy-5-(((((S)-5-oxopyrrolidin-2-yl)methyl)amino)methyl)pyridin-2-yl)-[1,1′-biphenyl]-3-yl)-8-fluoro-2-(2-((((S)-5-oxopyrrolidin-2-yl)methyl)amino)ethyl)-3,4-dihydroisoquinolin-1(2H)-one

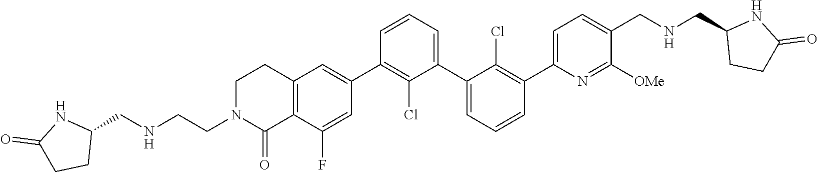 [SMILES: COc1nc(-c2cccc(-c3cccc(-c4cc(F)c5c(c4)CCN(CCNC[C@@H]4CCC(=O)N4)C5=O)c3Cl)c2Cl)ccc1CNC[C@@H]1CCC(=O)N1]

### 6-Bromo-2-(2,2-dimethoxyethyl)-8-fluoro-3,4-dihydroisoquinolin-1(2H)-one

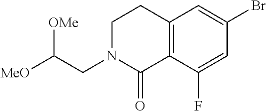 [SMILES: COC(CN1CCc2cc(Br)cc(F)c2C1=O)OC]

To a mixture of 6-bromo-8-fluoro-3,4-dihydroisoquinolin-1(2H)-one (800 mg, 3.28 mmol), 2-bromo-1,1-dimethoxyethane (1.11 g, 6.56 mmol) in THF/DMF mixture (5:1, 18 mL) was added potassium t-butoxide (552 mg, 4.92 mmol), tetrabutylammonium bromide (528 mg, 1.64 mmol). The mixture was stirred at 70° C. for 12 h under N₂. The reaction was quenched with water (30 mL) and extracted with ethyl acetate (2×30 mL). The combined organic phase was dried over anhydrous sodium sulfate, filtered and concentrated under reduced pressure to give a residue. The residue was purified by normal phase SiO₂ chromatography (0-40% EtOAc/petroleum ether) to afford 6-bromo-2-(2,2-dimethoxyethyl)-8-fluoro-3,4-dihydroisoquinolin-1(2H)-one (330 mg, 30% yield) as a yellow oil. ¹H NMR (400 MHz, DMSO-d₆): δ 7.47 (d, J=11.2 Hz, 1H), 7.41 (s, 1H), 4.51 (t, J=5.2 Hz, 1H), 3.51 (d, J=5.2 Hz, 4H), 3.29 (s, 6H), 2.91 (d, J=6.4 Hz, 2H).

### 6-(2,2′-Dichloro-3′-(2-(2,2-dimethoxyethyl)-8-fluoro-1-oxo-1,2,3,4-tetrahydroisoquinolin-6-yl)-[1,1′-biphenyl]-3-yl)-2-methoxynicotinaldehyde

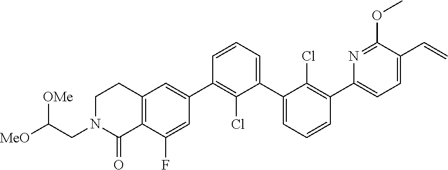 [SMILES: C=Cc1ccc(-c2cccc(-c3cccc(-c4cc(F)c5c(c4)CCN(CC(OC)OC)C5=O)c3Cl)c2Cl)nc1OC]

To a mixture of 6-bromo-2-(2,2-dimethoxyethyl)-8-fluoro-3,4-dihydroisoquinolin-1(2H)-one (170 mg, 0.51 mmol), 6-(2,2′-dichloro-3′-(4,4,5,5-tetramethyl-1,3,2-dioxaborolan-2-yl)-[1,1′-biphenyl]-3-yl)-2-methoxynicotinaldehyde (372 mg, 0.77 mmol) in dioxane/water mixture (5:1, 7.2 mL) was added [1,1-bis(diphenylphosphino)ferrocene]palladium(II) chloride dichloromethane complex (41.8 mg, 0.05 mmol) and potassium carbonate (212 mg, 1.54 mmol). The mixture was stirred at 110° C. for 0.5 h under N₂. The reaction was quenched with water (5 mL) and extracted with ethyl acetate (2×20 mL). The combined organic phase was dried over anhydrous sodium sulfate, filtered and concentrated under reduced pressure to give a residue. The residue was purified by normal phase SiO₂ chromatography (0-70% EtOAc/petroleum ether) to afford 6-(2,2′-dichloro-3′-(2-(2,2-dimethoxyethyl)-8-fluoro-1-oxo-1,2,3,4-tetrahydroisoquinolin-6-yl)-[1,1′-biphenyl]-3-yl)-2-methoxynicotinaldehyde (250 mg, 80% yield) as a white solid. ¹H NMR (400 MHz, DMSO-d₆): δ 10.31 (s, 1H), 8.22 (d, J=7.6 Hz, 1H), 8.22 (d, J=7.6 Hz, 1H), 7.75 (dd, J₁=1.6 Hz, J₂=7.6 Hz, 1H), 7.63-7.48 (m, 6H), 7.28-7.25 (m, 2H), 4.57 (t, J=5.6 Hz, 1H), 4.07 (s, 3H), 3.62-3.57 (m, 4H), 3.33 (s, 6H), 3.01 (t, J=6 Hz, 2H).

### 6-(2,2′-Dichloro-3′-(8-fluoro-1-oxo-2-(2-oxoethyl)-1,2,3,4-tetrahydroisoquinolin-6-yl)-[1,1′-biphenyl]-3-yl)-2-methoxynicotinaldehyde

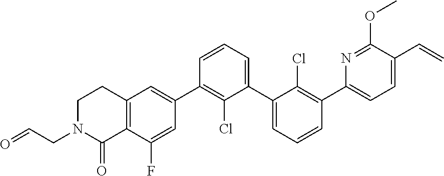 [SMILES: C=Cc1ccc(-c2cccc(-c3cccc(-c4cc(F)c5c(c4)CCN(CC=O)C5=O)c3Cl)c2Cl)nc1OC]

To a mixture of 6-(2,2′-dichloro-3′-(2-(2,2-dimethoxyethyl)-8-fluoro-1-oxo-1,2,3,4-tetrahydroisoquinolin-6-yl)-[1,1′-biphenyl]-3-yl)-2-methoxynicotinaldehyde (150 mg, 0.25 mmol) in DCM/H₂O mixture (10:1, 5.5 mL) was added trifluoroacetic acid (2.5 mL, 33.8 mmol), and then the mixture was stirred at rt for 0.5 h. The mixture was concentrated to afford 6-(2,2′-dichloro-3′-(8-fluoro-1-oxo-2-(2-oxoethyl)-1,2,3,4-tetrahydroisoquinolin-6-yl)-[1,1′-biphenyl]-3-yl)-2-methoxynicotinaldehyde (138 mg, crude, m/z: 565 [M+H]⁺ observed) as a yellow oil. The crude product was used directly without further purification.

### 6-(2,2′-Dichloro-3′-(6-methoxy-5-(((((S)-5-oxopyrrolidin-2-yl)methyl)amino)methyl)pyridin-2-yl)-[1,1′-biphenyl]-3-yl)-8-fluoro-2-(2-((((S)-5-oxopyrrolidin-2-yl)methyl)amino)ethyl)-3,4-dihydroisoquinolin-1(2H)-one

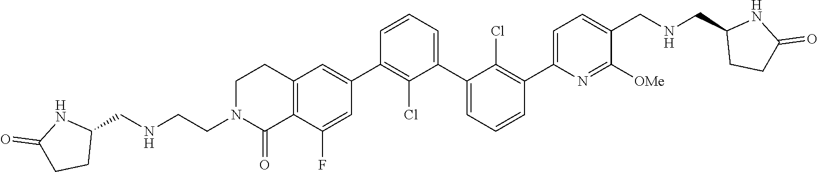 [SMILES: COc1nc(-c2cccc(-c3cccc(-c4cc(F)c5c(c4)CCN(CCNC[C@@H]4CCC(=O)N4)C5=O)c3Cl)c2Cl)ccc1CNC[C@@H]1CCC(=O)N1]

To a mixture of 6-[2-chloro-3-[2-chloro-3-[8-fluoro-1-oxo-2-(2-oxoethyl)-3,4-dihydroisoquinolin-6-yl]phenyl]phenyl]-2-methoxy-pyridine-3-carbaldehyde (138 mg, 0.24 mmol), (S)-5-(aminomethyl)pyrrolidin-2-one, HCl (148 mg, 0.98 mmol) in DCM (10 mL) was added TEA (0.68 mL, 4.90 mmol) and 4A molecular sieves (30 mg). The mixture was stirred at rt for 1.5 h under N₂. Sodium cyanoborohydride (61.6 mg, 0.98 mmol) was added and the mixture was stirred at rt for 0.5 h under N₂. The reaction mixture was filtered and concentrated under reduced pressure. The residue was purified by reverse phase HPLC to afford 6-(2,2′-dichloro-3′-(6-methoxy-5-(((((S)-5-oxopyrrolidin-2-yl)methyl)amino)methyl)pyridin-2-yl)-[1,1′-biphenyl]-3-yl)-8-fluoro-2-(2-((((S)-5-oxopyrrolidin-2-yl)methyl)amino)ethyl)-3,4-dihydroisoquinolin-1(2H)-one (84.6 mg, m/z: 759 [M+H]⁺ observed) as a white solid. ¹H NMR (400 MHz, DMSO-d₆): δ 7.83 (d, J=7.2 Hz, 1H), 7.69-7.66 (m, 3H), 7.56-7.45 (m, 5H), 7.29-7.23 (m, 3H), 3.93 (s, 3H), 3.74 (s, 2H), 3.68-3.51 (m, 4H), 3.05-3.02 (m, 2H), 2.81 (s, 2H), 2.62 (d, J=5.2 Hz, 2H), 2.58 (d, J=6 Hz, 1H), 2.15-2.06 (m, 6H), 1.72-1.67 (m, 2H).

### Example 401: (S)-5-((((2-(2,2′-Dichloro-3′-(6-methoxy-5-(((((S)-5-oxopyrrolidin-2-yl)methyl)amino) methyl)pyridin-2-yl)-[1,1′-biphenyl]-3-yl)-7-methyl-7H-pyrrolo[2,3-d]pyrimidin-5-yl)methyl)amino)methyl)pyrrolidin-2-one

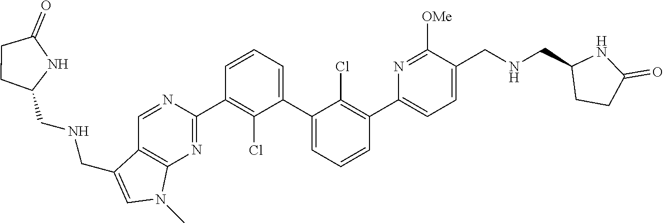 [SMILES: COc1nc(-c2cccc(-c3cccc(-c4ncc5c(CNC[C@@H]6CCC(=O)N6)cn(C)c5n4)c3Cl)c2Cl)ccc1CNC[C@@H]1CCC(=O)N1]

### 2-Chloro-7-methyl-7H-pyrrolo[2,3-d]pyrimidine-5-carbaldehyde

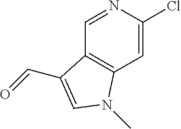

To a solution of 2-chloro-7H-pyrrolo[2,3-d]pyrimidine-5-carbaldehyde (0.3 g, 1.5 mmol) in THE (3 mL) at 0° C., sodium hydride (74 mg, 2.5 mmol) was added. After stirring for 15 min, iodomethane (0.28 mg, 2 mmol) was added at rt and the mixture was stirred for 12 h under N₂ atmosphere. The mixture was quenched with water (5 mL) and extracted with EtOAc (3×10 mL). The combined organic layer was dried over anhydrous sodium sulfate, filtered and concentrated under reduced pressure to afford 2-chloro-7-methyl-7H-pyrrolo[2,3-d]pyrimidine-5-carbaldehyde (0.3 g, 92% yield, m/z: 196 [M+H]⁺, observed). The crude product was used as is without purification.

### (S)-tert-Butyl ((6-(2,2′-dichloro-3′-(5-formyl-7-methyl-7H-pyrrolo[2,3-d]pyrimidin-2-yl)-[1,1′-biphenyl]-3-yl)-2-methoxypyridin-3-yl)methyl)((5-oxopyrrolidin-2-yl)methyl) carbamate

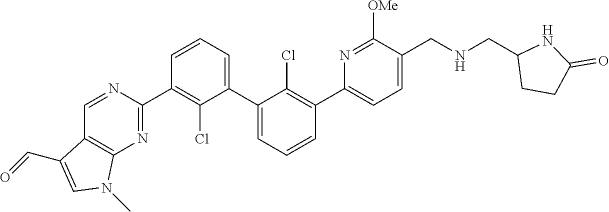 [SMILES: COc1nc(-c2cccc(-c3cccc(-c4ncc5c(C=O)cn(C)c5n4)c3Cl)c2Cl)ccc1CNCC1CCC(=O)N1]

A mixture of 2-chloro-7-methyl-pyrrolo[2,3-d]pyrimidine-5-carbaldehyde (0.28 g, 1 mmol), tert-butyl N-[[6-[2-chloro-3-[2-chloro-3-(4,4,5,5-tetramethyl-1,3,2-dioxaborolan-2-yl)phenyl]phenyl]-2-methoxy-3-pyridyl]methyl]-N-[[(2S)-5-oxopyrrolidin-2-yl]methyl]carbamate (0.95 g, 1 mmol), tris(dibenzylideneacetone)dipalladium(0) (0.13 g, 0.14 mmol), tricyclohexylphosphane (40 mg, 0.14 mmol) and potassium carbonate (0.6 g, 4 mmol) in dioxane/water mixture (10:1, 11 mL) was stirred at 120° C. for 3 h under N₂ atmosphere. The reaction mixture was concentrated, and the residue was suspended in water (15 mL). The aqueous layer was extracted with EtOAc (2×25 mL). The combined organic phase was dried with anhydrous sodium sulfate, filtered and concentrated to give a residue. The residue was purified by normal phase SiO₂ chromatography (0-40% PE/EtOAc) to afford (S)-tert-butyl ((6-(2,2′-dichloro-3′-(5-formyl-7-methyl-7H-pyrrolo[2,3-d]pyrimidin-2-yl)-[1,1′-biphenyl]-3-yl)-2-methoxypyridin-3-yl)methyl)((5-oxopyrrolidin-2-yl)methyl)carbamate (0.4 g, m/z: 715 [M+H]⁺ observed) as a brown solid.

### tert-Butyl ((6-(2,2′-dichloro-3′-(7-methyl-5-(((((S)-5-oxopyrrolidin-2-yl)methyl)amino) methyl)-7H-pyrrolo[2,3-d]pyrimidin-2-yl)-[1,1′-biphenyl]-3-yl)-2-methoxypyridin-3-yl)methyl)(((S)-5-oxopyrrolidin-2-yl)methyl)carbamate

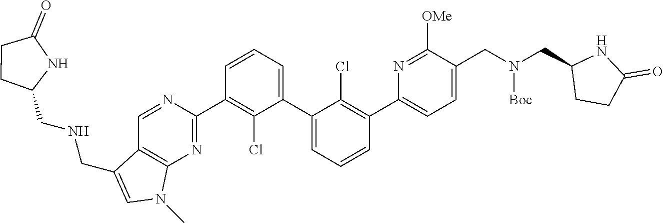 [SMILES: COc1nc(-c2cccc(-c3cccc(-c4ncc5c(CNC[C@@H]6CCC(=O)N6)cn(C)c5n4)c3Cl)c2Cl)ccc1CN(C[C@@H]1CCC(=O)N1)C(=O)OC(C)(C)C]

A mixture of tert-butyl N-[[6-[2-chloro-3-[2-chloro-3-(5-formyl-7-methyl-pyrrolo[2,3-d]pyrimidin-2-yl)phenyl]phenyl]-2-methoxy-3-pyridyl]methyl]-N-[[(2S)-5-oxopyrrolidin-2-yl]methyl]carbamate (0.4 g, 0.5 mmol), (5S)-5-(aminomethyl)pyrrolidin-2-one, HCl (0.12 g, 0.8 mmol), sodium acetate (0.13 g, 1.7 mmol) in DCM/MeOH mixture (1:1, 20 mL) was stirred at rt for 12 h. Sodium cyanoborohydride (70 mg, 1 mmol) was added and the mixture was stirred at rt for 1 h. The reaction mixture was filtered and concentrated under reduced pressure to afford tert-butyl ((6-(2,2′-dichloro-3′-(7-methyl-5-(((((S)-5-oxopyrrolidin-2-yl)methyl)amino)methyl)-7H-pyrrolo[2,3-d]pyrimidin-2-yl)-[1,1′-biphenyl]-3-yl)-2-methoxypyridin-3-yl)methyl)(((S)-5-oxopyrrolidin-2-yl)methyl)carbamate (0.6 g, crude, m/z: 813 [M+H]⁺, observed) as a yellow liquid.

### (S)-5-((((2-(2,2′-Dichloro-3′-(6-methoxy-5-(((((S)-5-oxopyrrolidin-2-yl)methyl)amino) methyl)pyridin-2-yl)-[1,1′-biphenyl]-3-yl)-7-methyl-7H-pyrrolo[2,3-d]pyrimidin-5-yl)methyl)amino)methyl)pyrrolidin-2-one

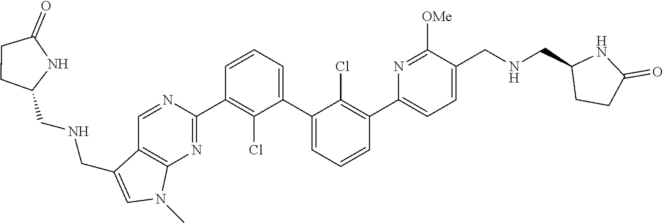 [SMILES: COc1nc(-c2cccc(-c3cccc(-c4ncc5c(CNC[C@@H]6CCC(=O)N6)cn(C)c5n4)c3Cl)c2Cl)ccc1CNC[C@@H]1CCC(=O)N1]

To a mixture of tert-butyl N-[[6-[2-chloro-3-[2-chloro-3-[7-methyl-5-[[[(2S)-5-oxopyrrolidin-2-yl]methylamino]methyl]pyrrolo[2,3-d]pyrimidin-2-yl]phenyl]phenyl]-2-methoxy-3-pyridyl]methyl]-N-[[(2S)-5-oxopyrrolidin-2-yl]methyl]carbamate (0.6 g, crude) in DCM (5 mL) was added trifluoroacetic acid (9 mL). The mixture was stirred at rt for 1 h. The reaction mixture was concentrated and the residue was purified by prep-HPLC to afford (S)-5-((((2-(2,2′-dichloro-3′-(6-methoxy-5-(((((S)-5-oxopyrrolidin-2-yl)methyl)amino)methyl)pyridin-2-yl)-[1,1′-biphenyl]-3-yl)-7-methyl-7H-pyrrolo[2,3-d]pyrimidin-5-yl)methyl)amino)methyl)pyrrolidin-2-one (50 mg, 12% yield, m/z: 713 [M+H]⁺, observed) was obtained as a white solid. ¹H NMR (400 MHz, DMSO-d₆): δ9.21 (s, 1H), 8.13 (s, 1H), 7.82 (d, J=6.8 Hz, 1H), 7.71-7.65 (m, 4H), 7.54-7.52 (m, 3H), 7.48-7.42 (m, 3H), 7.27 (d, J=7.6 Hz, 1H), 3.99-3.98 (m, 1H), 3.91 (d, J=1.6 Hz, 3H), 3.80 (s, 3H), 3.72 (s, 2H), 3.63 (br s, 3H), 2.62 (d, J=5.6 Hz, 2H), 2.56 (d, J=5.2 Hz, 2H), 2.10-2.08 (m, 7H), 1.70-1.67 (m, 3H).

### Example 402: (S)-5-((((6-(2,2′-Dichloro-3′-(1-methyl-3-(((((S)-5-oxopyrrolidin-2-yl)methyl)amino)methyl)-1H-pyrrolo[3,2-c]pyridin-6-yl)-[1,1′-biphenyl]-3-yl)-2-methoxypyridin-3-yl)methyl)amino) methyl)pyrrolidin-2-one

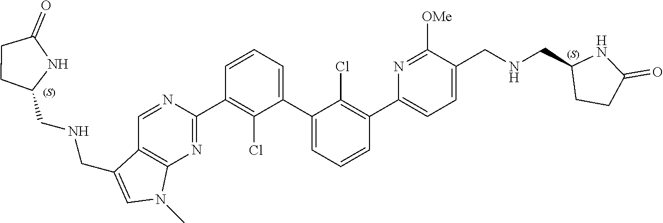

### 6-Chloro-1-methyl-1H-pyrrolo[3,2-c]pyridine-3-carbaldehyde

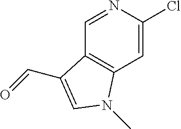

To a solution of 6-chloro-1H-pyrrolo[3,2-c]pyridine-3-carbaldehyde (0.5 g, 3 mmol) in DMF (3 mL) at 0° C., sodium hydride (0.12 g, 4 mmol) was added. After stirring for 15 min, iodomethane (0.2 mL, 3 mmol) was added at rt and the reaction mixture was stirred for 1.5 h under N₂. The reaction was quenched with water (8 mL) and extracted with EtOAc (3×15 mL). The combined organic phase was dried with anhydrous sodium sulfate, filtered and concentrated to give a residue. The residue was purified by normal phase SiO₂ chromatography (0-5% DCM/MeOH) to afford 6-chloro-1-methyl-1H-pyrrolo[3,2-c]pyridine-3-carbaldehyde (0.32 g, m/z: 195 [M+H]⁺ observed) as a yellow solid. 6-(2,2′-Dichloro-3′-(5-formyl-6-methoxypyridin-2-yl)-[1,1′-biphenyl]-3-yl)-1-methyl-1H-pyrrolo[3,2-c]pyridine-3-carbaldehyde

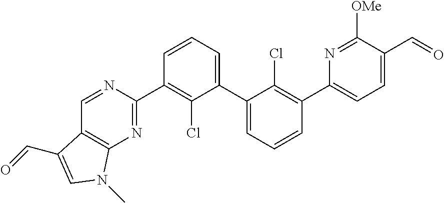 [SMILES: COc1nc(-c2cccc(-c3cccc(-c4ncc5c(C=O)cn(C)c5n4)c3Cl)c2Cl)ccc1C=O]

A mixture of 6-chloro-1-methyl-pyrrolo[3,2-b]pyridine-3-carbaldehyde (0.15 g, 0.8 mmol), 6-[2-chloro-3-[2-chloro-3-(4,4,5,5-tetramethyl-1,3,2-dioxaborolan-2-yl)phenyl]phenyl]-2-methoxy-pyridine-3-carbaldehyde (0.8 g, 1.7 mmol), tris(dibenzylideneacetone)dipalladium(0) (0.07 g, 0.08 mmol), tricyclohexylphosphane (0.02 g, 0.09 mmol) and potassium carbonate (0.32 g, 2 mmol) in dioxane/water mixture (10:1, 3.3 mL) was stirred at 120° C. for 12 h under N₂. The reaction mixture was concentrated, and the residue was purified by normal phase SiO₂ chromatography (0-50% EtOAc/petroleum ether) to afford 6-(2,2′-dichloro-3′-(5-formyl-6-methoxypyridin-2-yl)-[1,1′-biphenyl]-3-yl)-1-methyl-1H-pyrrolo[3,2-c]pyridine-3-carbaldehyde (0.13 g, 32% yield, m/z: 516[M+H]⁺, observed) as a yellow solid.

### (S)-5-((((6-(2,2′-Dichloro-3′-(1-methyl-3-(((((S)-5-oxopyrrolidin-2-yl)methyl)amino) methyl)-1H-pyrrolo[3,2-c]pyridin-6-yl)-[1,1′-biphenyl]-3-yl)-2-methoxypyridin-3-yl)methyl)amino)methyl)pyrrolidin-2-one

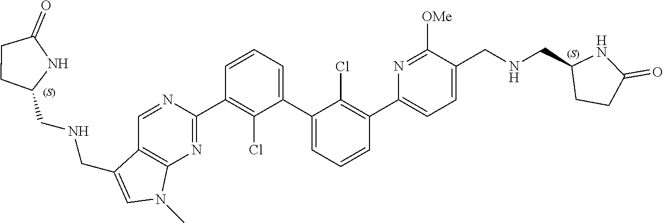

A mixture of 6-[2-chloro-3-[2-chloro-3-(5-formyl-6-methoxy-2-pyridyl)phenyl]phenyl]-1-methyl-pyrrolo[3,2-c]pyridine-3-carbaldehyde (45 mg, 87 umol), (5S)-5-(aminomethyl) pyrrolidin-2-one, HCl (40 mg, 0.26 mmol) and sodium acetate (40 mg, 0.5 mmol) in DCM/MeOH mixture (1:1, 1 mL) was stirred at rt for 12 h. Sodium cyanoborohydride (22 mg, 0.35 mol) was added and the stirring was continued for 1 h. The reaction mixture was concentrated and the residue was purified by prep-HPLC to afford (S)-5-((((6-(2,2′-dichloro-3′-(1-methyl-3-(((((S)-5-oxopyrrolidin-2-yl)methyl)amino)methyl)-1H-pyrrolo[3,2-c]pyridin-6-yl)-[1,1′-biphenyl]-3-yl)-2-methoxypyridin-3-yl)methyl)amino)methyl)pyrrolidin-2-one (7.4 mg, 10% yield, m/z: 712[M+H]⁺, observed) as a white solid. ¹H NMR (400 MHz, DMSO-d₆): δ8.99 (s, 1H), 8.14 (s, 2H), 7.81 (d, J=8.4 Hz, 1H), 7.68-7.64 (m, 4H), 7.61-7.59 (m, 1H), 7.56-7.48 (m, 2H), 7.44-7.37 (m, 3H), 7.26 (d, J=7.6 Hz, 1H), 3.97 (s, 2H), 3.90 (s, 3H), 3.78 (s, 3H), 3.70 (s, 2H), 3.61 (br s, 4H), 2.65-2.53 (m, 2H), 2.15-2.04 (m, 8H), 1.67-1.64 (m, 3H).

### Example 407: 8-(2,2′-Dichloro-3′-(6-methoxy-5-(((((S)-5-oxopyrrolidin-2-yl)methyl)amino)methyl)pyridin-2-yl)-[1,1′-biphenyl]-3-yl)-3-(((((1S,2R)-2-(hydroxymethyl)cyclopropyl)methyl)amino) methyl)-4H-pyrido[1,2-a]pyrimidin-4-one

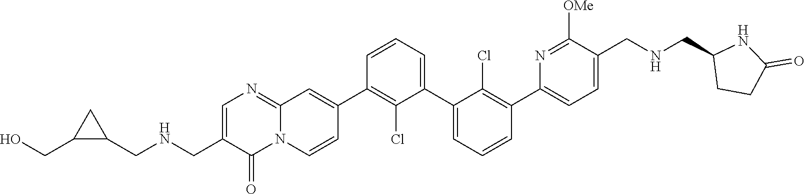 [SMILES: COc1nc(-c2cccc(-c3cccc(-c4ccn5c(=O)c(CNCC6CC6CO)cnc5c4)c3Cl)c2Cl)ccc1CNC[C@@H]1CCC(=O)N1]

### tert-Butyl ((6-(2,2′-dichloro-3′-(3-((((2-(hydroxymethyl)cyclopropyl)methyl)amino)methyl)-4-oxo-4H-pyrido[1,2-a]pyrimidin-8-yl)-[1,1′-biphenyl]-3-yl)-2-methoxypyridin-3-yl)methyl)(((S)-5-oxopyrrolidin-2-yl)methyl)carbamate

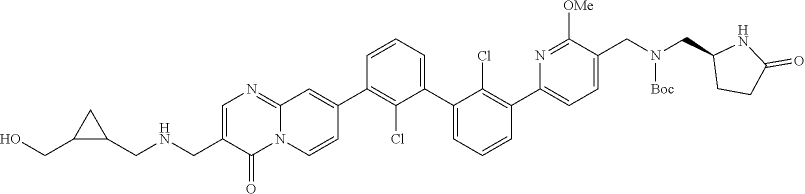 [SMILES: COc1nc(-c2cccc(-c3cccc(-c4ccn5c(=O)c(CNCC6CC6CO)cnc5c4)c3Cl)c2Cl)ccc1CN(C[C@@H]1CCC(=O)N1)C(=O)OC(C)(C)C]

To a mixture of (S)-tert-butyl ((6-(2,2′-dichloro-3′-(3-formyl-4-oxo-4H-pyrido[1,2-a]pyrimidin-8-yl)-[1,1′-biphenyl]-3-yl)-2-methoxypyridin-3-yl)methyl)((5-oxopyrrolidin-2-yl)methyl) carbamate (960 mg, 1.32 mmol) in DCM (10 mL) was added ((1R,2S)-2-(aminomethyl) cyclopropyl)methanol (199.9 mg, 1.98 mmol) and NaOAc (540 mg, 6.59 mmol). After 1 h, NaBH₃CN (124.2 mg, 1.98 mmol) was added and the mixture was stirred for 11 h at rt. The solvent was evaporated and the residue was purified by normal phase SiO₂ chromatography (0-100% EtOAc/petroleum ether to 0-25% MeOH/EtOAc) to afford tert-butyl ((6-(2,2′-dichloro-3′-(3-((((2-(hydroxymethyl)cyclopropyl)methyl)amino)methyl)-4-oxo-4H-pyrido[1,2-a]pyrimidin-8-yl)-[1,1′-biphenyl]-3-yl)-2-methoxypyridin-3-yl)methyl)(((S)-5-oxopyrrolidin-2-yl)methyl) carbamate as a yellow solid (0.74 g, 60% yield).

### 8-(2,2′-Dichloro-3′-(6-methoxy-5-(((((S)-5-oxopyrrolidin-2-yl)methyl)amino)methyl)pyridin-2-yl)-[1,1′-biphenyl]-3-yl)-3-(((((1S,2R)-2-(hydroxymethyl)cyclopropyl)methyl)amino) methyl)-4H-pyrido[1,2-a]pyrimidin-4-one

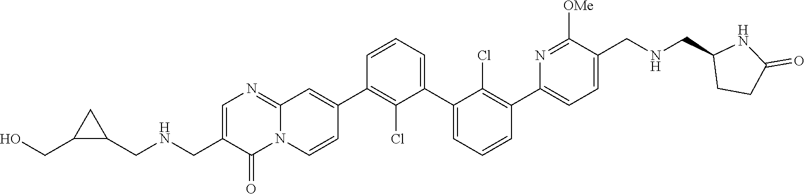 [SMILES: COc1nc(-c2cccc(-c3cccc(-c4ccn5c(=O)c(CNCC6CC6CO)cnc5c4)c3Cl)c2Cl)ccc1CNC[C@@H]1CCC(=O)N1]

To a solution of tert-butyl ((6-(2,2′-dichloro-3′-(3-((((2-(hydroxymethyl)cyclopropyl) methyl) amino)methyl)-4-oxo-4H-pyrido[1,2-a]pyrimidin-8-yl)-[1,1′-biphenyl]-3-yl)-2-methoxypyridin-3-yl)methyl)(((S)-5-oxopyrrolidin-2-yl)methyl) carbamate (0.74 g, 0.91 mmol) in DCM (8 mL) was added 2,2,2-trifluoroacetic acid (2 mL, 27.0 mmol) and stirred at rt for 0.5 h. The mixture was concentrated, and the residue was purified by reverse phase HPLC to afford 8-(2,2′-dichloro-3′-(6-methoxy-5-(((((S)-5-oxopyrrolidin-2-yl)methyl)amino)methyl) pyridin-2-yl)-[1,1′-biphenyl]-3-yl)-3-(((((1S,2R)-2-(hydroxymethyl)cyclopropyl)methyl)amino) methyl)-4H-pyrido[1,2-a]pyrimidin-4-one (220 mg, 0.29 mmol). m/z: 713 [M+H]⁺ observed). ¹H NMR (400 MHz, DMSO-d₆): δ 8.87 (d, J=7.6 Hz, 1H), 8.26 (s, 1H), 8.04 (s, 1H), 7.67 (d, J=7.6 Hz, 1H), 7.64 (d, J=1.2 Hz, 1H), 7.53-7.51 (m, 3H), 7.48 (t, J=7.2 Hz, 1H), 7.42-7.38 (m, 2H), 7.36 (dd, J=7.6 Hz, J=2.0 Hz, 1H), 7.31 (dd, J=7.6 Hz, J=1.6 Hz, 1H), 7.12 (d, J=7.2 Hz, 1H), 3.76 (s, 3H), 3.72-3.63 (m, 3H), 3.56-3.46 (m, 3H), 3.00-2.94 (m, 2H), 2.78-2.72 (m, 1H), 2.40 (d, J=6.0 Hz, 1H), 1.99-1.91 (m, 3H), 1.56-1.52 (m, 1H), 0.99-0.95 (m, 2H), 0.54-0.49 (m, 1H).

### Example 408: 8-(2,2′-Dichloro-3′-(6-methoxy-5-(((((S)-5-oxopyrrolidin-2-yl)methyl)amino)methyl)pyridin-2-yl)-[1,1′-biphenyl]-3-yl)-3-(((((1S,2R)-2-(hydroxymethyl)cyclopropyl)methyl) amino) methyl)-4H-pyrido[1,2-a]pyrimidin-4-one

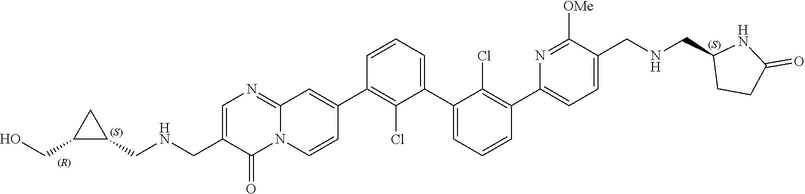

### Example 409: 8-(2,2′-Dichloro-3′-(6-methoxy-5-(((((S)-5-oxopyrrolidin-2-yl)methyl)amino)methyl)pyridin-2-yl)-[1,1′-biphenyl]-3-yl)-3-(((((1R,2S)-2-(hydroxymethyl)cyclopropyl)methyl)amino) methyl)-4H-pyrido[1,2-a]pyrimidin-4-one

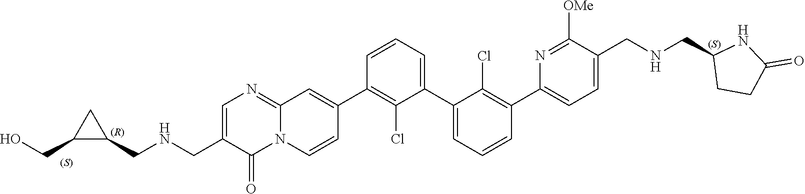

126 mg of the mixture of enantiomers was separated by SFC (supercritical fluid chromatography) on a DAICEL CHIRALPAK IF (250 mm*30 mm,10 um)column using liquid MeOH and ACN (50%; 0.1% aqueous NH₃ as modifier) to give8-(2,2′-dichloro-3′-(6-methoxy-5-(((((S)-5-oxopyrrolidin-2-yl)methyl)amino)methyl)pyridin-2-yl)-[1,1′-biphenyl]-3-yl)-3-(((((1S,2R)-2-(hydroxymethyl)cyclopropyl)methyl)amino)methyl)-4H-pyrido[1,2-a]pyrimidin-4-one (4.8 mg, 3% yield) as a yellow solid and 8-(2,2′-dichloro-3′-(6-methoxy-5-(((((S)-5-oxopyrrolidin-2-yl)methyl)amino)methyl)pyridin-2-yl)-[1,1′-biphenyl]-3-yl)-3-(((((1S,2R)-2-(hydroxymethyl)cyclopropyl)methyl)amino)methyl)-4H-pyrido[1,2-a]pyrimidin-4-one (6.7 mg, 5% yield).

### Example 408: 8-(2,2′-Dichloro-3′-(6-methoxy-5-(((((S)-5-oxopyrrolidin-2-yl)methyl)amino)methyl)pyridin-2-yl)-[1,1′-biphenyl]-3-yl)-3-(((((1S,2R)-2-(hydroxymethyl)cyclopropyl)methyl)amino) methyl)-4H-pyrido[1,2-a]pyrimidin-4-one

m/z: 713 [M+H]⁺ observed. ¹H NMR (400 MHz, DMSO-d₆): δ 8.93 (d, J=7.6 Hz, 1H), 7.74 (d, J=7.6 Hz, 1H), 7.68 (d, J=1.6 Hz, 1H), 7.60-7.58 (m, 3H), 7.54 (t, J=7.2 Hz, 1H), 7.49-7.46 (m, 2H), 7.41-7.37 (m, 2H), 7.19 (d, J=7.2 Hz, 1H), 3.83 (s, 3H), 3.69-3.59 (m, 5H), 3.57-3.53 (m, 2H), 3.06-3.01 (m, 1H), 2.77-2.72 (m, 1H), 2.06-1.99 (m, 3H), 1.67-1.59 (m, 1H), 1.15 (s, 2H), 1.01-0.96 (m, 2H), 0.80-0.72 (m, 1H), 0.58-0.53 (m, 1H), 0.03-0.02 (m, 1H). SFC, Rt=1.785 min on OD column.

### Example 409: 8-(2,2′-Dichloro-3′-(6-methoxy-5-(((((S)-5-oxopyrrolidin-2-yl)methyl)amino)methyl)pyridin-2-yl)-[1,1′-biphenyl]-3-yl)-3-(((((1S,2R)-2-(hydroxymethyl)cyclopropyl)methyl)amino) methyl)-4H-pyrido[1,2-a]pyrimidin-4-one

m/z: 713 [M+H]⁺ observed. ¹H NMR (400 MHz, DMSO-d₆): δ 8.92 (d, J=8.0 Hz, 1H), 8.29 (s, 1H), 7.73 (d, J=7.6 Hz, 1H), 7.67 (d, J=1.2 Hz, 1H), 7.60-7.57 (m, 3H), 7.53 (t, J=7.6 Hz, 1H), 7.48-7.44 (m, 2H), 7.40-7.36 (m, 2H), 7.18 (d, J=7.2 Hz, 1H), 3.82 (s, 3H), 3.68-3.58 (m, 3H), 3.56-3.51 (m, 2H), 3.05-3.00 (m, 1H), 2.76-2.72 (m, 1H), 2.25-2.22 (m, 2H), 2.05-1.96 (m, 1H), 1.65-1.58 (m, 1H), 1.16-1.14 (m, 1H), 0.99-0.95 (m, 2H), 0.76-0.74 (m, 1H), 10.57-0.52 (m, 1H). SFC, Rt=10.13 min on OD column.

### Example 418: (S)-5-((((6-(2,2′-Dichloro-3′-(2-((S)-2-hydroxypropyl)-8-methoxy-1,2,3,4 tetrahydroisoquinolin-6-yl)-[1,1′-biphenyl]-3-yl)-2-methoxypyridin-3-yl)methyl)amino) methyl)pyrrolidin-2-one

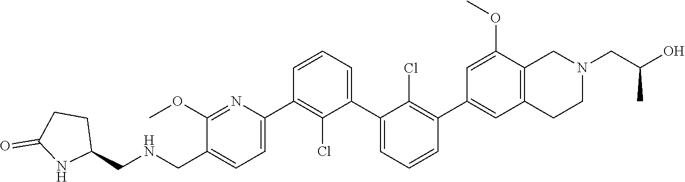 [SMILES: COc1cc(-c2cccc(-c3cccc(-c4ccc(CNC[C@@H]5CCC(=O)N5)c(OC)n4)c3Cl)c2Cl)cc2c1CN(C[C@H](C)O)CC2]

### (S)-1-(6-Bromo-8-methoxy-3,4-dihydroisoquinolin-2(1H)-yl)propan-2-ol

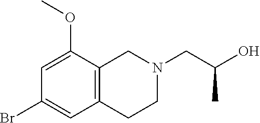 [SMILES: COc1cc(Br)cc2c1CN(C[C@H](C)O)CC2]

To a mixture of 6-bromo-8-methoxy-1,2,3,4-tetrahydroisoquinoline, HCl (0.12 g, 0.43 mmol) in EtOH (2 mL) was added N-ethyl-N-isopropylpropan-2-amine (0.45 mL, 2.58 mmol) and (S)-2-methyloxirane (0.15 g, 2.58 mmol) under N₂ atmosphere. The mixture was stirred at rt for 12 h. The mixture was concentrated to afford the (S)-1-(6-bromo-8-methoxy-3,4-dihydroisoquinolin-2(1H)-yl)propan-2-ol as a yellow gum (0.23 g, crude, m/z: 300 [M]⁺). The material was used as is without any purification.

### tert-Butyl ((6-(2,2′-dichloro-3′-(2-((S)-2-hydroxypropyl)-8-methoxy-1,2,3,4-tetrahydroisoquinolin-6-yl)-[1,1′-biphenyl]-3-yl)-2-methoxypyridin-3-yl)methyl)(((S)-5-oxopyrrolidin-2-yl)methyl)carbamate

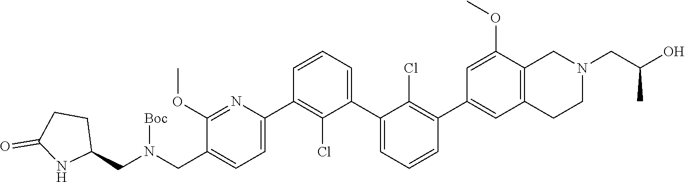 [SMILES: COc1cc(-c2cccc(-c3cccc(-c4ccc(CN(C[C@@H]5CCC(=O)N5)C(=O)OC(C)(C)C)c(OC)n4)c3Cl)c2Cl)cc2c1CN(C[C@H](C)O)CC2]

To a mixture of (S)-1-(6-bromo-8-methoxy-3,4-dihydroisoquinolin-2(1H)-yl)propan-2-ol (0.19 g, 0.63 mmol) and (S)-tert-butyl ((6-(2,2′-dichloro-3′-(4,4,5,5-tetramethyl-1,3,2-dioxaborolan-2-yl)-[1,1′-biphenyl]-3-yl)-2-methoxypyridin-3-yl)methyl)((5-oxopyrrolidin-2-yl)methyl) carbamate (0.39 g, 0.57 mmol) in THF/water mixture (10:1, 6.6 mL) was added potassium phosphate (0.27 g, 1.27 mmol) and [1,1′-Bis(di-tert-butylphosphino)ferrocene]dichloropalladium(II) (0.04 g, 0.06 mmol). The mixture was stirred at 80° C. for 2 h. The mixture was concentrated, and the residue was suspended in water (10 mL) and brine (10 mL). The mixture was extracted with ethyl acetate/THF mixture (1:1, 50 mL). The organic phase was dried with anhydrous sodium sulfate, filtered and concentrated. The residue was purified by normal phase SiO₂ chromatography (0-100% ethyl acetate/petroleum ether) to afford tert-butyl ((6-(2,2′-dichloro-3′-(2-((S)-2-hydroxypropyl)-8-methoxy-1,2,3,4-tetrahydroisoquinolin-6-yl)-[1,1′-biphenyl]-3-yl)-2-methoxypyridin-3-yl)methyl)(((S)-5-oxopyrrolidin-2-yl)methyl)carbamate (0.2 g) m/z: 775 [M+H]⁺ observed.

### (S)-5-((((6-(2,2′-Dichloro-3′-(2-((S)-2-hydroxypropyl)-8-methoxy-1,2,3,4-tetrahydroisoquinolin-6-yl)-[1,1′-biphenyl]-3-yl)-2-methoxypyridin-3-yl)methyl)amino) methyl)pyrrolidin-2-one

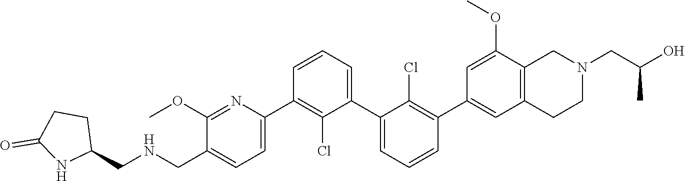 [SMILES: COc1cc(-c2cccc(-c3cccc(-c4ccc(CNC[C@@H]5CCC(=O)N5)c(OC)n4)c3Cl)c2Cl)cc2c1CN(C[C@H](C)O)CC2]

To a mixture of tert-butyl ((6-(2,2′-dichloro-3′-(2-((S)-2-hydroxypropyl)-8-methoxy-1,2,3,4-tetrahydroisoquinolin-6-yl)-[1,1′-biphenyl]-3-yl)-2-methoxypyridin-3-yl)methyl)(((S)-5-oxopyrrolidin-2-yl)methyl)carbamate (0.18 g, 0.23 mmol) in CH₂Cl₂ (1.5 mL) was added trifluoroacetic acid (0.8 mL, 10.8 mmol). The mixture was stirred at rt for 0.5 h. The mixture was concentrated and the residue was purified by reverse phase HPLC to afford (S)-5-((((6-(2,2′-dichloro-3′-(2-((S)-2-hydroxypropyl)-8-methoxy-1,2,3,4-tetrahydroisoquinolin-6-yl)-[1,1′-biphenyl]-3-yl)-2-methoxypyridin-3-yl)methyl)amino)methyl)pyrrolidin-2-one (36 mg, 13% yield for three steps, m/z: 675 [M+H]⁺ observed) as a yellow solid. ¹H NMR (400 MHz, DMSO-d₆): δ 7.83 (d, J=7.6 Hz, 1H), 7.71-7.65 (m, 2H), 7.54-7.39 (m, 5H), 7.28 (d, J=7.6 Hz, 1H), 6.81 (d, J=9.2 Hz, 2H), 4.00-3.85 (m, 5H), 3.81 (s, 3H), 3.78-3.39 (m, 4H), 2.84-2.83 (m, 2H), 2.73-2.72 (m, 2H), 2.57 (d, J=6.4 Hz, 2H), 2.48-2.38 (m, 2H), 2.18-2.05 (m, 3H), 1.75-1.67 (m, 1H), 1.09 (d, J=6 Hz, 3H).

### Example 419: (S)-5-((((6-(2,2′-Dichloro-3′-(2-((R)-2-hydroxypropyl)-8-methoxy-1,2,3,4-tetrahydroisoquinolin-6-yl)-[1,1′-biphenyl]-3-yl)-2-methoxypyridin-3-yl)methyl)amino) methyl)pyrrolidin-2-one

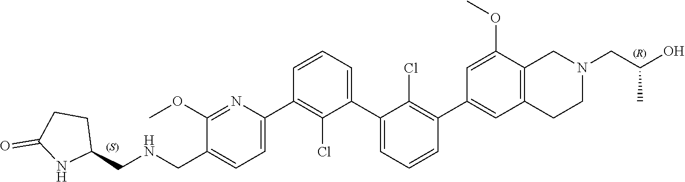

### (R)-1-(6-Bromo-8-methoxy-3,4-dihydroisoquinolin-2(1H)-yl)propan-2-ol

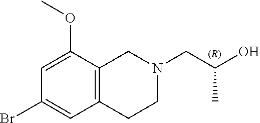

To a solution of 6-bromo-8-methoxy-1,2,3,4-tetrahydroisoquinoline, HCl (200 mg, 0.72 mmol) in EtOH (2 mL) was added (2R)-2-methyloxirane (0.25 mL, 3.59 mmol) and triethylamine (0.5 mL, 3.59 mmol). The mixture was stirred at rt for 12 h. The reaction mixture was concentrated to give (R)-1-(6-bromo-8-methoxy-3,4-dihydroisoquinolin-2(1H)-yl)propan-2-ol (350 mg, crude, m/z: 300 [M+], 302 [M+2]⁺ observed) as a yellow oil. The material was used as is without any purification.

### tert-Butyl ((6-(2,2′-dichloro-3′-(2-((R)-2-hydroxypropyl)-8-methoxy-1,2,3,4-tetrahydroisoquinolin-6-yl)-[1,1′-biphenyl]-3-yl)-2-methoxypyridin-3-yl)methyl)(((S)-5-oxopyrrolidin-2-yl)methyl)carbamate

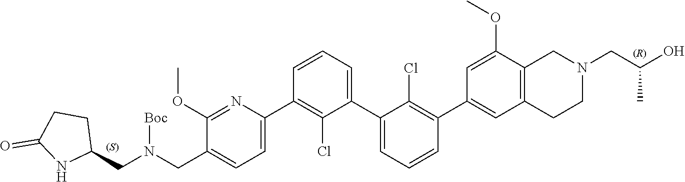

To a solution of (R)-1-(6-bromo-8-methoxy-3,4-dihydroisoquinolin-2(1H)-yl)propan-2-ol (330 mg, 1.10 mmol) and (S)-tert-butyl ((6-(2,2′-dichloro-3′-(4,4,5,5-tetramethyl-1,3,2-dioxaborolan-2-yl)-[1,1′-biphenyl]-3-yl)-2-methoxypyridin-3-yl)methyl)((5-oxopyrrolidin-2-yl)methyl)carbamate (750 mg, 1.10 mmol) in dioxane (5 mL) and water (1 mL) was added 1,1′-bis(diphenylphosphino)ferrocene-palladium(ii) dichloride dichloromethane complex (90 mg, 0.11 mmol) and potassium carbonate (304 mg, 2.20 mmol). The mixture was stirred at 100° C. for 2 h. The reaction mixture was diluted with water (10 mL) and extracted with ethyl acetate (2×10 mL). The combined organic layers were dried over anhydrous sodium sulfate, filtered and concentrated. The residue was purified by normal phase SiO₂ chromatography (0-100% ethyl acetate/petroleum ether) to afford tert-butyl ((6-(2,2′-dichloro-3′-(2-((R)-2-hydroxypropyl)-8-methoxy-1,2,3,4-tetrahydroisoquinolin-6-yl)-[1,1′-biphenyl]-3-yl)-2-methoxypyridin-3-yl)methyl)(((S)-5-oxopyrrolidin-2-yl)methyl)carbamate (250 mg, 29% yield, m/z: 775 [M+H]⁺ observed) as a white solid.

### (S)-5-((((6-(2,2′-Dichloro-3′-(2-((R)-2-hydroxypropyl)-8-methoxy-1,2,3,4-tetrahydroisoquinolin-6-yl)-[1,1′-biphenyl]-3-yl)-2-methoxypyridin-3-yl)methyl)amino) methyl)pyrrolidin-2-one

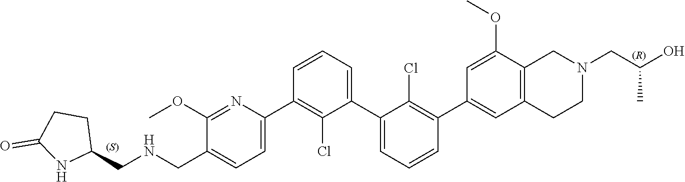

To a solution of tert-butyl ((6-(2,2′-dichloro-3′-(2-((R)-2-hydroxypropyl)-8-methoxy-1,2,3,4-tetrahydroisoquinolin-6-yl)-[1,1′-biphenyl]-3-yl)-2-methoxypyridin-3-yl)methyl)(((S)-5-oxopyrrolidin-2-yl)methyl)carbamate (230 mg, 0.30 mmol) in DCM (3 mL) was added trifluoroacetic acid (1 mL, 13.5 mmol). The mixture was stirred at rt for 30 min. The reaction mixture was concentrated and the residue was purified by reverse phase HPLC to afford (S)-5-((((6-(2,2′-dichloro-3′-(2-((R)-2-hydroxypropyl)-8-methoxy-1,2,3,4-tetrahydroisoquinolin-6-yl)-[1,1′-biphenyl]-3-yl)-2-methoxypyridin-3-yl)methyl)amino) methyl)pyrrolidin-2-one (36.5 mg, 17% yield, m/z: 675 [M+H]⁺ observed) as a white solid. ¹H NMR (400 MHz, DMSO-d₆): δ 7.74 (d, J=7.2 Hz, 1H), 7.63 (dd, J=7.6 Hz, J=1.6 Hz, 1H), 7.50-7.31 (m, 5H), 7.25 (d, J=7.2 Hz, 1H), 6.84-6.81 (m, 2H), 4.42-4.38 (m, 1H), 3.93 (s, 3H), 3.90-3.82 (m, 1H), 3.81 (s, 3H), 3.73-3.54 (m, 5H), 2.85-2.81 (m, 2H), 2.71-2.68 (m, 2H), 2.58-2.56 (m, 2H), 2.42-2.37 (m, 2H), 2.16-2.09 (m, 3H), 1.76-1.67 (m, 1H), 1.09 (d, J=6.0 Hz, 3H).

### Example 435: (S)-5-((((6-(2,2′-Dichloro-3′-((S)-5-((((S)-5-oxopyrrolidin-2-yl)methyl)amino)-5,6,7,8-tetrahydroquinolin-2-yl)-[1,1′-biphenyl]-3-yl)-2-methoxypyridin-3-yl)methyl)amino) methyl)pyrrolidin-2-one

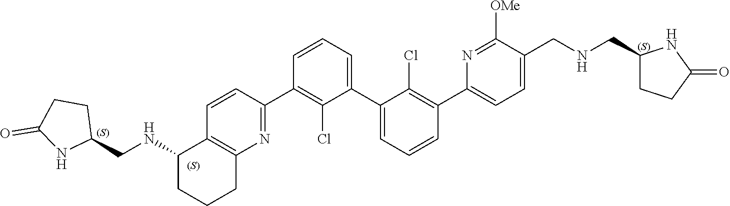

### (3R)-3-(((2-Chloro-5,6,7,8-tetrahydroquinolin-5-yl)amino)methyl)cyclopentanone

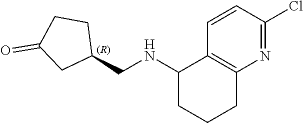

To a mixture of 2-chloro-7,8-dihydroquinolin-5(6H)-one (0.6 g, 3.3 mmol) and (S)-5-(aminomethyl)-2-pyrrolidinone (0.53 g, 4.63 mmol) in THE (20 mL) was added titanium(IV) ethoxide (2 mL, 9.91 mmol) in a sealed tube. The mixture was stirred at 105° C. for 16 h. The mixture was cooled to −20° C. Sodium borohydride (0.37 g, 9.91 mmol) was added, and the mixture was stirred at rt for 0.5 h. The mixture was quenched with saturated aqueous ammonium chloride solution (10 mL), ice water (10 mL) and MeOH (40 mL). The mixture was filtered. The filtrate was concentrated, and the residue was suspended in water (20 mL) and brine (10 mL). The mixture was extracted with ethyl acetate/THF mixture (1:1, 2×30 mL). The organic phase was dried with anhydrous sodium sulfate, filtered and concentrated. The residue was purified by normal phase SiO₂ chromatography (30-100% ethyl acetate/petroleum ether to 10% methanol/ethyl acetate) to afford (3R)-3-(((2-chloro-5,6,7,8-tetrahydroquinolin-5-yl)amino) methyl)cyclopentanone as yellow solid (0.68 g, 36% yield). ¹H NMR (400 MHz, Methanol-d₄): δ 7.94-7.90 (m, 1H), 7.25 (d, J=8.4 Hz, 1H), 3.97-3.76 (m, 2H), 2.94-2.65 (m, 4H), 2.42-2.25 (m, 3H), 2.11-1.94 (m, 2H), 1.91-1.76 (m, 3H).

### tert-Butyl ((S)-2-chloro-5,6,7,8-tetrahydroquinolin-5-yl)(((S)-5-oxopyrrolidin-2-yl)methyl) carbamate and tert-butyl ((R)-2-chloro-5,6,7,8-tetrahydroquinolin-5-yl)(((S)-5-oxopyrrolidin-2-yl)methyl)carbamate

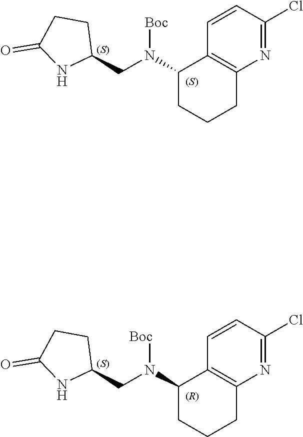

To a mixture of (5S)-5-[[(2-chloro-5,6,7,8-tetrahydroquinolin-5-yl)amino]methyl]pyrrolidin-2-one (0.15 g, 0.54 mmol), N-ethyl-N-isopropylpropan-2-amine (0.22 mL, 1.29 mmol), di-tert-butyl dicarbonate (0.53 g, 2.41 mmol) in THE (12 mL) was added 4-dimethylaminopyridine (0.1 g, 0.8 mmol) in one portion under N₂ atmosphere. The mixture was stirred at rt for 12 h. The mixture was diluted with water (20 mL) and brine (20 mL). The mixture was extracted with ethyl acetate (30 mL). The organic phase was dried with anhydrous sodium sulfate, filtered and concentrated. The residue was purified by normal phase SiO₂ chromatography (0-40% ethyl acetate/petroleum ether) to afford to afford 0.5 g of the racemic product. The racemate was further separated by SFC (column: DAICEL CHIRALCEL OX (250 mm*30 mm,10 um);mobile phase: [0.1% NH₃H₂O MeOH];B %: 25%-25%,min) to afford tert-butyl ((S)-2-chloro-5,6,7,8-tetrahydroquinolin-5-yl)(((S)-5-oxopyrrolidin-2-yl)methyl)carbamate (faster eluting enantiomer, 0.32 g) and tert-butyl ((R)-2-chloro-5,6,7,8-tetrahydroquinolin-5-yl)(((S)-5-oxopyrrolidin-2-yl)methyl)carbamate (slower eluting enantiomer, 0.23 g). 0.32 g of tert-butyl ((S)-2-chloro-5,6,7,8-tetrahydroquinolin-5-yl)(((S)-5-oxopyrrolidin-2-yl)methyl)carbamate was purified by prep-TLC (SiO₂, petroleum ether: ethyl acetate=1:2) to afford 0.15 g of tert-butyl ((S)-2-chloro-5,6,7,8-tetrahydroquinolin-5-yl)(((S)-5-oxopyrrolidin-2-yl)methyl)carbamate (faster eluting enantiomer, 0.15 g, 33% yield, m/z: 402 [M+Na]⁺ observed). 0.23 g of tert-butyl ((R)-2-chloro-5,6,7,8-tetrahydroquinolin-5-yl)(((S)-5-oxopyrrolidin-2-yl)methyl)carbamate was purified by prep-TLC (SiO₂, petroleum ether: ethyl acetate=1:2) to afford tert-butyl ((R)-2-chloro-5,6,7,8-tetrahydroquinolin-5-yl)(((S)-5-oxopyrrolidin-2-yl)methyl)carbamate(slower eluting enantiomer, 0.08 g, 17% yield, m/z: 402 [M+Na]⁺ observed).

### tert-Butyl N-[(5S)-2-[3-[3-[5-[[tert-butoxycarbonyl-[[(2S)-5-oxopyrrolidin-2-yl]methyl]amino]methyl]-6-methoxy-2-pyridyl]-2-chloro-phenyl]-2-chloro-phenyl]-5,6,7,8-tetrahydroquinolin-5-yl]-N-[[(2S)-5-oxopyrrolidin-2-yl]methyl]carbamate

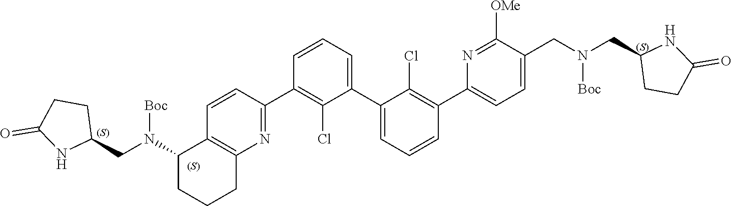

To a mixture of tert-butyl ((S)-2-chloro-5,6,7,8-tetrahydroquinolin-5-yl)(((S)-5-oxopyrrolidin-2-yl)methyl)carbamate (0.14 g, 0.37 mmol) and (S)-tert-butyl ((6-(2,2′-dichloro-3′-(4,4,5,5-tetramethyl-1,3,2-dioxaborolan-2-yl)-[1,1′-biphenyl]-3-yl)-2-methoxypyridin-3-yl)methyl)((5-oxopyrrolidin-2-yl)methyl)carbamate (0.4 g, 0.59 mmol) in dioxane/water mixture (10:1, 5.5 mL) was added [1,1′-bis(di-tert-butylphosphino)ferrocene]dichloropalladium(II) (0.02 g, 0.04 mmol) and potassium phosphate (0.23 g, 1.11 mmol) in one portion under N₂ atmosphere. The mixture was stirred at 110° C. for 1 h. The mixture was concentrated, and the residue was suspended in water (5 mL) and brine (5 mL). The mixture was extracted with ethyl acetate/THF mixture (1:1, 10 mL). The organic phase was dried with anhydrous sodium sulfate, filtered and concentrated. The residue was purified by normal phase SiO₂ chromatography (0-70% 50% THE in ethyl acetate/petroleum ether) to afford tert-butyl N-[(5S)-2-[3-[3-[5-[[tert-butoxycarbonyl-[[(2S)-5-oxopyrrolidin-2-yl]methyl]amino]methyl]-6-methoxy-2-pyridyl]-2-chloro-phenyl]-2-chloro-phenyl]-5,6,7,8-tetrahydroquinolin-5-yl]-N-[[(2S)-5-oxopyrrolidin-2-yl]methyl]carbamate as a yellow solid (0.14 g, 39% yield, m/z: 899 [M+H]⁺ observed).

### (S)-5-((((6-(2,2′-Dichloro-3′-((S)-5-((((S)-5-oxopyrrolidin-2-yl)methyl)amino)-5,6,7,8-tetrahydroquinolin-2-yl)-[1,1′-biphenyl]-3-yl)-2-methoxypyridin-3-yl)methyl)amino)methyl)pyrrolidin-2-one

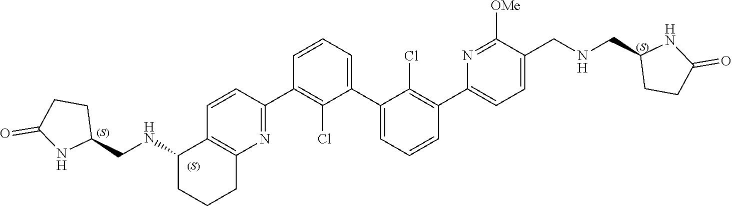

To a mixture of tert-butyl N-[(5S)-2-[3-[3-[5-[[tert-butoxycarbonyl-[[(2S)-5-oxopyrrolidin-2-yl]methyl]amino]methyl]-6-methoxy-2-pyridyl]-2-chloro-phenyl]-2-chloro-phenyl]-5,6,7,8-tetrahydroquinolin-5-yl]-N-[[(2S)-5-oxopyrrolidin-2-yl]methyl]carbamate (0.15 g, 0.17 mmol) in CH₂Cl₂ (1 mL) was added trifluoroacetic acid (1.5 mL, 20 mmol) in one portion under N₂ atmosphere. The mixture was stirred at rt for 1 h. The mixture was concentrated and the residue was purified by reverse phase HPLC to afford (S)-5-((((6-(2,2′-dichloro-3′-((S)-5-((((S)-5-oxopyrrolidin-2-yl)methyl)amino)-5,6,7,8-tetrahydroquinolin-2-yl)-[1,1′-biphenyl]-3-yl)-2-methoxypyridin-3-yl)methyl)amino)methyl)pyrrolidin-2-one as a white solid (15.5 mg, 12% yield, m/z: 699 [M+H]⁺ observed). ¹H NMR (400 MHz, DMSO-d₆): δ 7.94 (d, J=8 Hz, 1H), 7.85 (d, J=8 Hz, 1H), 7.68-7.66 (m, 3H), 7.57-7.51 (m, 3H), 7.46-7.44 (m, 3H), 7.30 (d, J=7.6 Hz, 1H), 3.94 (s, 3H), 3.82 (m, 3H), 3.69-3.63 (m, 3H), 2.93-2.85 (m, 3H), 2.68-2.63 (m, 5H), 2.16-1.93 (m, 9H), 1.79-1.71 (m, 4H).

### Example 456: (S)-5-((((6-(2,2′-Dichloro-3′-((R)-5-((((S)-5-oxopyrrolidin-2-yl)methyl)amino)-5,6,7,8-tetrahydroquinolin-2-yl)-[1,1′-biphenyl]-3-yl)-2-methoxypyridin-3-yl)methyl)amino) methyl)pyrrolidin-2-one

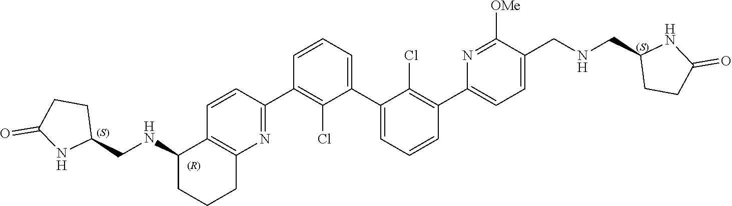

### tert-Butyl N-[(5R)-2-[3-[3-[5-[[tert-butoxycarbonyl-[[(2S)-5-oxopyrrolidin-2-yl]methyl]amino]methyl]-6-methoxy-2-pyridyl]-2-chloro-phenyl]-2-chloro-phenyl]-5,6,7,8-tetrahydroquinolin-5-yl]-N-[[(2S)-5-oxopyrrolidin-2-yl]methyl]carbamate

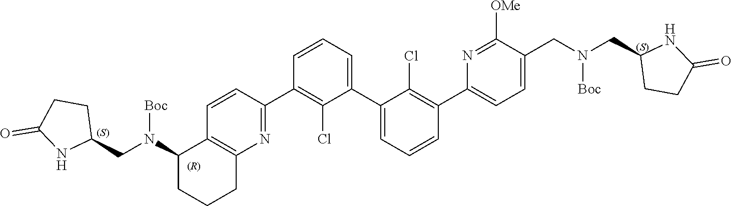

To a mixture of tert-butyl ((R)-2-chloro-5,6,7,8-tetrahydroquinolin-5-yl)(((S)-5-oxopyrrolidin-2-yl)methyl)carbamate (0.07 g, 0.14 mmol) and (S)-tert-butyl ((6-(2,2′-dichloro-3′-(4,4,5,5-tetramethyl-1,3,2-dioxaborolan-2-yl)-[1,1′-biphenyl]-3-yl)-2-methoxypyridin-3-yl)methyl)((5-oxopyrrolidin-2-yl)methyl)carbamate (0.25 g, 0.37 mmol) in dioxane/water mixture (8:1, 4.5 mL) was added [1,1′-bis(di-tert-butylphosphino)ferrocene]dichloropalladium(II) (0.01 g, 0.02 mmol) and potassium phosphate (0.12 g, 0.55 mmol) in one portion under N₂ atmosphere. The mixture was stirred at 110° C. for 1 h. The mixture was concentrated, and the residue was added water (5 mL) and brine (5 mL). The mixture was extracted with ethyl acetate/THF mixture (1:1, 10 mL). The organic phase was dried with anhydrous sodium sulfate, filtered and concentrated. The residue was purified by normal phase SiO₂ chromatography (0-70% 50% THE in ethyl acetate/petroleum ether) to afford tert-butyl N-[(5R)-2-[3-[3-[5-[[tert-butoxycarbonyl-[[(2S)-5-oxopyrrolidin-2-yl]methyl]amino]methyl]-6-methoxy-2-pyridyl]-2-chloro-phenyl]-2-chloro-phenyl]-5,6,7,8-tetrahydroquinolin-5-yl]-N-[[(2S)-5-oxopyrrolidin-2-yl]methyl]carbamate as a yellow solid (0.13 g, 45% yield, m/z: 899 [M+H]⁺ observed).

### (S)-5-((((6-(2,2′-Dichloro-3′-((R)-5-((((S)-5-oxopyrrolidin-2-yl)methyl)amino)-5,6,7,8-tetrahydroquinolin-2-yl)-[1,1′-biphenyl]-3-yl)-2-methoxypyridin-3-yl)methyl)amino) methyl)pyrrolidin-2-one

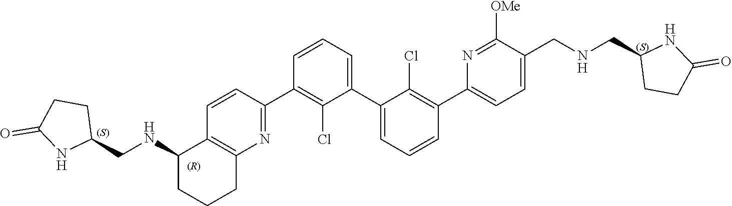

To a mixture of tert-butyl N-[(5R)-2-[3-[3-[5-[[tert-butoxycarbonyl-[[(2S)-5-oxopyrrolidin-2-yl]methyl]amino]methyl]-6-methoxy-2-pyridyl]-2-chloro-phenyl]-2-chloro-phenyl]-5,6,7,8-tetrahydroquinolin-5-yl]-N-[[(2S)-5-oxopyrrolidin-2-yl]methyl]carbamate (0.11 g, 0.04 mmol) in CH₂Cl₂ (1 mL) was added trifluoroacetic acid (2 mL, 27 mmol) in one portion under N₂ atmosphere. The mixture was stirred at rt for 1 h. The mixture was concentrated and the residue was purified by reverse phase HPLC to afford (S)-5-((((6-(2,2′-dichloro-3′-((R)-5-((((S)-5-oxopyrrolidin-2-yl)methyl)amino)-5, 6,7,8-tetrahydroquinolin-2-yl)-[1,1′-biphenyl]-3-yl)-2-methoxypyridin-3-yl)methyl)amino)methyl)pyrrolidin-2-one as a white solid (5.6 mg, 19% yield, m/z: 699 [M+H]⁺ observed). ¹H NMR (400 MHz, DMSO-d₆): δ 7.97 (d, J=8 Hz, 1H), 7.90 (d, J=7.2 Hz, 1H), 7.71-7.64 (m, 3H), 7.59-7.54 (m, 3H), 7.49-7.45 (m, 3H), 7.35 (d, J=7.2 Hz, 1H), 4.02-3.96 (m, 6H), 3.78 (m, 1H), 3.66 (m, 1H), 2.94-2.85 (m, 6H), 2.22-1.75 (m, 15H).

### Example 397: 6-(2,2′-Dichloro-3′-(6-methoxy-5-(((((S)-5-oxopyrrolidin-2-yl)methyl)amino)methyl)pyridin-2-yl)-[1,1′-biphenyl]-3-yl)-2-(2-((((S)-5-oxopyrrolidin-2-yl)methyl)amino)ethyl)-2,3-dihydro-1H-pyrrolo[3,4-c]pyridin-1-one

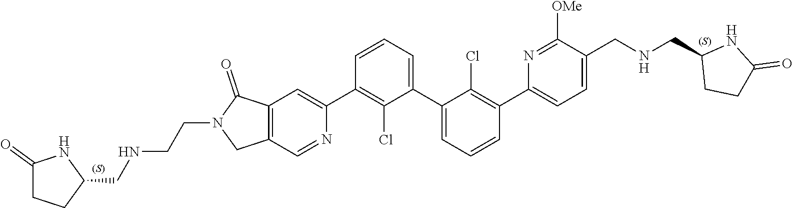

### Methyl 2-chloro-5-methylisonicotinate

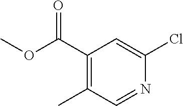

To a mixture of methyl 5-bromo-2-chloroisonicotinate (3.26 g, 13 mmol), methyl boronic acid (1.56 g, 26 mmol), potassium phosphate (6.91 g, 32.5 mmol) in water (3 mL) and dioxane (30 mL) was added [1,1′-bis(di-tert-butylphosphino)ferrocene]dichloropalladium(II) (0.85 g, 1.30 mmol). The mixture was degassed and purged with N₂ and stirred at 110° C. for 0.5 h under N₂ atmosphere. The reaction was combined with another batch at 5 g scale. The reaction mixture was extracted with saturated aqueous ammonium chloride solution (50 mL) and EtOAc (3×30 mL). The combined organic layers were washed with brine (2×30 mL), dried over anhydrous sodium sulfate, filtered and concentrated under reduced pressure to give a residue. The residue was purified by normal phase SiO₂ chromatography (0-100% EtOAc/petroleum ether) to afford methyl 2-chloro-5-methylisonicotinate as a yellow oily liquid (4.5 g). ¹H NMR (400 MHz, DMSO-d₆): δ 8.43 (s, 1H), 7.72 (s, 1H), 3.85 (s, 1H), 2.42 (s, 1H).

### Methyl 5-(bromomethyl)-2-chloroisonicotinate

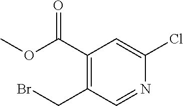

To a mixture of methyl 2-chloro-5-methylisonicotinate (4 g, 21.55 mmol) and n-bromosuccinimide (4.60 g, 25.9 mmol) in CHCl₃ (40 mL) was added AIBN (0.35 g, 2.16 mmol). The mixture was degassed and purged with N₂ and stirred at 70° C. for 12 h. The reaction mixture was diluted with water 50 ml and the aqueous was extracted with EtOAc (3×30 mL). The combined organic layers were washed with bine (2×50 mL), dried over anhydrous sodium sulfate, filtered and concentrated under reduced pressure. The residue was purified by normal phase SiO₂ chromatography (0-100% EtOAc/petroleum ether) to afford methyl 5-(bromomethyl)-2-chloroisonicotinate as a yellow liquid (4.24 g, 62% yield)¹H NMR (400 MHz, DMSO-d₆): δ 8.78 (s, 1H), 7.97 (s, 1H), 5.02 (s, 2H), 4.06 (s, 3H).

### 6-Chloro-2-(2,2-dimethoxyethyl)-2,3-dihydro-1H-pyrrolo[3,4-c]pyridin-1-one

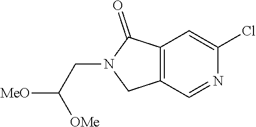 [SMILES: COC(CN1Cc2cnc(Cl)cc2C1=O)OC]

To a mixture of methyl 5-(bromomethyl)-2-chloroisonicotinate (4.24 g, 16. mmol) in MeOH (18 mL) was added 2,2-dimethoxyethanamine (5.79 g, 55.1 mmol). The mixture was degassed and purged with N₂ and stirred at 25° C. for 3 h. The reaction mixture was filtered and concentrated under reduced pressure. The residue was extracted with water 20 ml and EtOAc (3×25 mL). The combined organic layers were washed with brine (2×20 mL), dried over anhydrous sodium sulfate, filtered and concentrated under reduced pressure. The residue was purified by normal phase SiO₂ chromatography (0-100% EtOAc/petroleum ether) to afford 6-chloro-2-(2,2-dimethoxyethyl)-2,3-dihydro-1H-pyrrolo[3,4-c]pyridin-1-one as a white solid (3 g, 59% yield). ¹H NMR (400 MHz, DMSO-d₆): δ 8.71 (s, 1H), 7.75 (s, 1H), 4.61 (s, 2H), 4.57 (t, J=5.2 Hz, 1H), 3.63 (d, J=5.2 Hz, 2H), (3.28 (s, 6H).

### 6-(2,2′-Dichloro-3′-(2-(2,2-dimethoxyethyl)-1-oxo-2,3-dihydro-1H-pyrrolo[3,4-c]pyridin-6-yl)-[1,1′-biphenyl]-3-yl)-2-methoxynicotinaldehyde

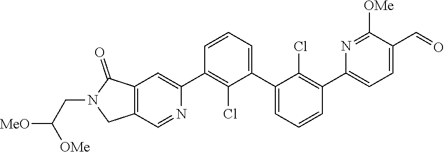 [SMILES: COc1nc(-c2cccc(-c3cccc(-c4cc5c(cn4)CN(CC(OC)OC)C5=O)c3Cl)c2Cl)ccc1C=O]

To a mixture of 6-chloro-2-(2,2-dimethoxyethyl)-2,3-dihydro-1H-pyrrolo[3,4-c]pyridin-1-one (0.5 g, 1.95 mmol), 6-(2,2′-dichloro-3′-(4,4,5,5-tetramethyl-1,3-dioxolan-2-yl)-[1,1′-biphenyl]-3-yl)-2-methoxynicotinaldehyde (1.32 g, 2.73 mmol) and potassium carbonate (0.67 g, 4.87 mmol) in dioxane (10 mL) and water (1 mL) was added 1,1′-bis(diphenylphosphino)ferrocene-palladium(ii) dichloride dichloromethane complex (0.16 g, 0.19 mmol). The mixture was degassed and purged with N₂ and stirred at 110° C. for 0.5 h. The reaction mixture was diluted with water 30 ml and the aqueous layer was extracted with ethyl acetate (3×30 mL). The combined organic layers were washed with brine (2×30 mL), dried over anhydrous sodium sulfate, filtered and concentrated under reduced pressure. The residue was purified by normal phase SiO₂ chromatography (0-100% EtOAc/petroleum ether) to afford 6-(2,2′-dichloro-3′-(2-(2,2-dimethoxyethyl)-1-oxo-2,3-dihydro-1H-pyrrolo[3,4-c]pyridin-6-yl)-[1,1′-biphenyl]-3-yl)-2-methoxynicotinaldehyde as a yellow solid. (500 mg, 38% yield m/z: 578.2 [M+H]⁺ observed)

### 6-(2,2′-Dichloro-3′-(1-oxo-2-(2-oxoethyl)-2,3-dihydro-1H-pyrrolo[3,4-c]pyridin-6-yl)-[1,1′-biphenyl]-3-yl)-2-methoxynicotinaldehyde

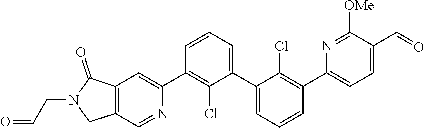 [SMILES: COc1nc(-c2cccc(-c3cccc(-c4cc5c(cn4)CN(CC=O)C5=O)c3Cl)c2Cl)ccc1C=O]

A solution 6-(2,2′-dichloro-3′-(2-(2,2-dimethoxyethyl)-1-oxo-2,3-dihydro-1H-pyrrolo[3,4-c]pyridin-6-yl)-[1,1′-biphenyl]-3-yl)-2-methoxynicotinaldehyde (0.12 g, 0.21 mmol) and iron(iii)chloridehexahydrate (0.16 g, 0.58 mmol) in DCM (0.8 mL) and acetonitrile (0.2 mL) was stirred at 45° C. for 2 h. The reaction mixture was diluted with water (10 mL), and the aqueous layer was extracted with DCM (2×10 ml). The combined organic layers were dried over anhydrous sodium sulfate, filtered and dried to give 6-(2,2′-dichloro-3′-(1-oxo-2-(2-oxoethyl)-2,3-dihydro-1H-pyrrolo[3,4-c]pyridin-6-yl)-[1,1′-biphenyl]-3-yl)-2-methoxynicotinaldehyde (m/z: 532.1, [M+H]⁺ observed). The crude product was used directly without further purification

### 6-(2,2′-Dichloro-3′-(6-methoxy-5-(((((S)-5-oxopyrrolidin-2-yl)methyl)amino)methyl)pyridin-2-yl)-[1,1′-biphenyl]-3-yl)-2-(2-((((S)-5-oxopyrrolidin-2-yl)methyl)amino)ethyl)-2,3-dihydro-1H-pyrrolo[3,4-c]pyridin-1-one

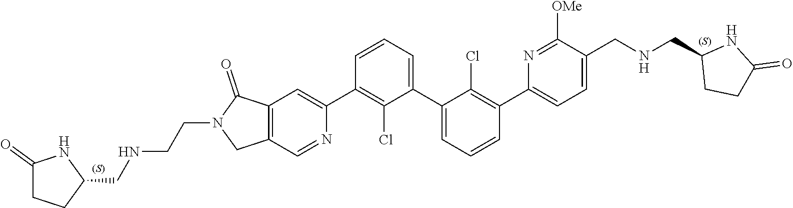

To a solution of 6-(2,2′-dichloro-3′-(1-oxo-2-(2-oxoethyl)-2,3-dihydro-1H-pyrrolo[3,4-c]pyridin-6-yl)-[1,1′-biphenyl]-3-yl)-2-methoxynicotinaldehyde in DCM (25 mL) and MeOH (5 mL) was added (S)-5-(aminomethyl)pyrrolidin-2-one, HCl (0.12 g, 1.09 mmol). The mixture was stirred at 25° C. for 20 min. Sodium cyanoborohydride (0.03 g, 0.41 mmol) was added and the mixture was stirred at 25° C. for 10 min. The reaction mixture was filtered and concentrated under reduced pressure. The residue was purified by reverse phase HPLC to afford 6-(2,2′-dichloro-3′-(6-methoxy-5-(((((S)-5-oxopyrrolidin-2-yl)methyl)amino)methyl)pyridin-2-yl)-[1,1′-biphenyl]-3-yl)-2-(2-((((S)-5-oxopyrrolidin-2-yl)methyl)amino)ethyl)-2,3-dihydro-1H-pyrrolo[3,4-c]pyridin-1-one as a white solid (16.9 mg, 10% yield, m/z: 728.3[M+H]⁺ observed. ¹H NMR (400 MHz, DMSO-d₆): δ 9.04 (s, 1H), 8.15 (s, 1H), 7.87-7.83 (m, 2H), 7.71-7.65 (m, 4H), 7.59-7.48 (m, 4H), 7.30 (d, J=7.6 Hz, 1H), 4.73 (s, 2H), 3.93 (s, 3H), 3.80-3.75 (m, 2H), 3.67-3.58 (m, 5H), 2.88 (t, J=6.0 Hz, 2H), 2.61-2.59 (m, 3H), 2.18-2.03 (m, 6H), 1.73-1.63 (m, 2H)

### Example 455: (S)-5-((((6-(2,2′-Dichloro-3′-(2-(2-hydroxyethyl)-8-methoxy-1,2,3,4-tetrahydroisoquinolin-6-yl)-[1,1′-biphenyl]-3-yl)-2-methoxypyridin-3-yl)methyl)amino)methyl)pyrrolidin-2-one

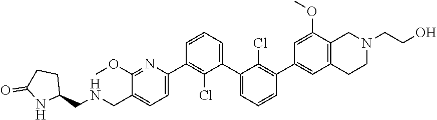 [SMILES: COc1cc(-c2cccc(-c3cccc(-c4ccc(CNC[C@@H]5CCC(=O)N5)c(OC)n4)c3Cl)c2Cl)cc2c1CN(CCO)CC2]

### 6-bromo-2-(2-((tert-Butyldimethylsilyl)oxy)ethyl)-8-methoxy-1,2,3,4-tetrahydroisoquinoline

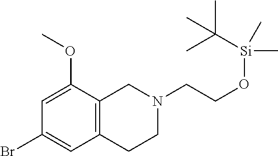 [SMILES: COc1cc(Br)cc2c1CN(CCO[Si](C)(C)C(C)(C)C)CC2]

To a mixture of 6-bromo-8-methoxy-1,2,3,4-tetrahydroisoquinoline (0.01 g, 0.41 mmol) and 2-bromoethoxy-tert-butyl-dimethyl-silane (0.15 g, 0.62 mmol) in acetonitrile (3 mL) was added triethylamine (0.28 mL, 2.07 mmol) in one portion. The mixture was stirred at 100° C. for 15 h under N₂ atmosphere. The mixture was concentrated, and the residue was suspended in water (5 mL). The mixture was extracted with ethyl acetate(5 mL). The organic phase was dried with anhydrous sodium sulfate, filtered and concentrated to afford 2-(6-bromo-8-methoxy-3,4-dihydro-1H-isoquinolin-2-yl)ethoxy-tert-butyl-dimethyl-silane as a yellow gum (0.14 g, crude, m/z: 400 [M]⁺). The material was used as is without any purification.

### (S)-tert-Butyl ((6-(3′-(2-(2-((tert-butyldimethylsilyl)oxy)ethyl)-8-methoxy-1,2,3,4-tetrahydroisoquinolin-6-yl)-2,2′-dichloro-[1,1′-biphenyl]-3-yl)-2-methoxypyridin-3-yl)methyl)((5-oxopyrrolidin-2-yl)methyl)carbamate

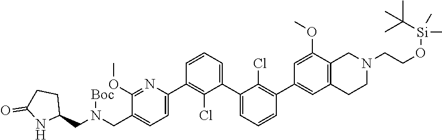 [SMILES: COc1cc(-c2cccc(-c3cccc(-c4ccc(CN(C[C@@H]5CCC(=O)N5)C(=O)OC(C)(C)C)c(OC)n4)c3Cl)c2Cl)cc2c1CN(CCO[Si](C)(C)C(C)(C)C)CC2]

To a mixture of 2-(6-bromo-8-methoxy-3,4-dihydro-1H-isoquinolin-2-yl)ethoxy-tert-butyl-dimethyl-silane (0.24 g, 0.6 mmol) and tert-butyl N-[[6-[2-chloro-3-[2-chloro-3-(4,4,5,5-tetramethyl-1,3,2-dioxaborolan-2-yl)phenyl]phenyl]-2-methoxy-3-pyridyl]methyl]-N-[[(2S)-5-oxopyrrolidin-2-yl]methyl]carbamate (0.61 g, 0.9 mmol) in dioxane/water mixture (50:7, 5.7 mL) was added potassium phosphate (0.38 g, 1.8 mmol) and 1,1′-bis(diphenylphosphino) ferrocene-palladium(ii) dichloride dichloromethane complex (0.31 g, 0.48 mmol) in one portion. The mixture was stirred at 110° C. for 1 h. To the residue was added water (10 mL) and brine solution (10 mL). The mixture was extracted with ethyl acetate (20 mL). The organic phase was dried with anhydrous sodium sulfate, filtered and concentrated to give the residue. The residue was purified by normal phase SiO₂ chromatography (0-60% ethyl acetate/petroleum ether) to afford tert-butyl N-[[6-[3-[3-[2-[2-[tert-butyl(dimethyl)silyl]oxyethyl]-8-methoxy-3,4-dihydro-1H-isoquinolin-6-yl]-2-chloro-phenyl]-2-chloro-phenyl]-2-methoxy-3-pyridyl]methyl]-N-[[(2S)-5-oxopyrrolidin-2-yl]methyl]carbamate as a white solid (0.39 g, 70% yield, m/z: 875 [M+H]⁺ observed).

### (S)-5-((((6-(2,2′-Dichloro-3′-(2-(2-hydroxyethyl)-8-methoxy-1,2,3,4-tetrahydroisoquinolin-6-yl)-[1,1′-biphenyl]-3-yl)-2-methoxypyridin-3-yl)methyl)amino)methyl)pyrrolidin-2-one

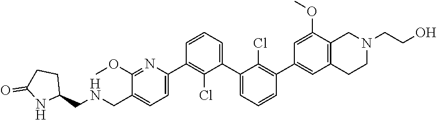 [SMILES: COc1cc(-c2cccc(-c3cccc(-c4ccc(CNC[C@@H]5CCC(=O)N5)c(OC)n4)c3Cl)c2Cl)cc2c1CN(CCO)CC2]

To a mixture of tert-butyl N-[[6-[3-[3-[2-[2-[tert-butyl(dimethyl)silyl]oxyethyl]-8-methoxy-3,4-dihydro-1H-isoquinolin-6-yl]-2-chloro-phenyl]-2-chloro-phenyl]-2-methoxy-3-pyridyl]methyl]-N-[[(2S)-5-oxopyrrolidin-2-yl]methyl]carbamate (0.36 g, 0.41 mmol) in CH₂Cl₂ (1 mL) was added trifluoroacetic acid (2 mL, 27 mmol) in one portion. The mixture was stirred at rt for 0.5 h. The mixture was concentrated, and the residue was purified by reverse phase HPLC to afford (5S)-5-[[[6-[2-chloro-3-[2-chloro-3-[2-(2-hydroxyethyl)-8-methoxy-3,4-dihydro-1H-isoquinolin-6-yl]phenyl]phenyl]-2-methoxy-3-pyridyl]methylamino]methyl]pyrrolidin-2-one as a white solid (0.04 g, 13% yield, m/z: 661 [M+H]⁺ observed). ¹H NMR (400 MHz, DMSO-d₆): δ 8.17 (s, 1H), 7.84 (d, J=7.2 Hz, 1H), 7.70-7.66 (m, 2H), 7.57-7.40 (m, 5H), 7.29 (d, J=7.2 Hz, 1H), 6.83 (d, J=9.6 Hz, 1H), 3.94 (s, 3H), 3.82 (s, 3H), 3.77-3.76 (m, 3H), 3.67-3.60 (m, 10H), 2.86-2.85 (m, 2H), 2.78-2.77 (m, 2H), 2.67 (t, J=6 Hz, 2H), 2.60 (d, J=6.4 Hz, 2H), 2.19-2.07 (m, 3H), 1.77-1.68 (m, 1H).

### Example 467: 6-(2,2′-Dichloro-3′-(6-methoxy-5-(((((S)-5-oxopyrrolidin-2-yl)methyl)amino)methyl)pyridin-2-yl)-[1,1′-biphenyl]-3-yl)-2-(2-((((S)-5-oxopyrrolidin-2-yl)methyl)amino)ethyl)-1H-pyrrolo[3,4-c]pyridin-3(2H)-one

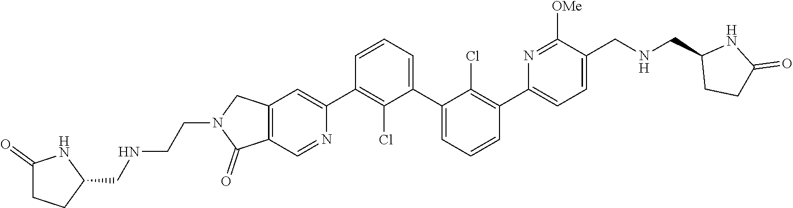 [SMILES: COc1nc(-c2cccc(-c3cccc(-c4cc5c(cn4)C(=O)N(CCNC[C@@H]4CCC(=O)N4)C5)c3Cl)c2Cl)ccc1CNC[C@@H]1CCC(=O)N1]

### Methyl 4-(bromomethyl)-6-chloronicotinate

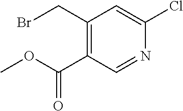

To a mixture of methyl 6-chloro-4-methylnicotinate (4 g, 21 mmol) in CHCl₃ (90 mL) was added N-Bromosuccinimide (4.99 g, 28 mmol) and 2,2-azobis(2-methylpropionitrile) (0.35 g, 2.16 mmol) in one portion under N₂ atmosphere. The mixture was stirred at 70° C. for 16 h. To the mixture was added water (5 mL). The organic phase was washed with brine solution (2×5 mL), dried with anhydrous sodium sulfate, filtered and concentrated. The residue was purified by normal phase SiO₂ chromatography (0-10% ethyl acetate/petroleum ether) to afford methyl 4-(bromomethyl)-6-chloronicotinate as a colorless oil (7 g, m/z: 266 [M+H]⁺ observed).

### 6-Chloro-2-(2,2-dimethoxyethyl)-1H-pyrrolo[3,4-c]pyridin-3(2H)-one

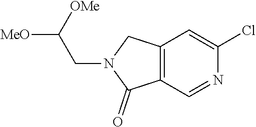 [SMILES: COC(CN1Cc2cc(Cl)ncc2C1=O)OC]

To a mixture of methyl 4-(bromomethyl)-6-chloronicotinate (7 g, 10.6 mmol) in MeOH (40 mL) was added 2,2-dimethoxyethanamine (3.5 mL, 31.8 mmol) in one portion under N₂ atmosphere. The mixture was stirred at rt for 1 h. The mixture was concentrated, and the residue was suspended in water (20 mL) and brine solution (5 mL). The mixture was extracted with ethyl acetate/THF mixture (1:1, 20 mL). The organic phase was dried with anhydrous sodium sulfate, filtered and concentrated. The residue was purified by normal phase SiO₂ chromatography (0-40% ethyl acetate/petroleum ether) to afford 6-chloro-2-(2,2-dimethoxyethyl)-1H-pyrrolo[3,4-c]pyridin-3(2H)-one as a white solid (1.2 g, 44% yield). ¹H NMR (400 MHz, CDCl₃): δ 8.79 (s, 1H), 7.38 (s, 1H), 4.49 (s, 2H), 4.47 (t, J=5.2 Hz, 1H), 3.65 (d, J=4.8 Hz, 2H), 3.36 (s, 6H).

### 2-(6-Chloro-3-oxo-1H-pyrrolo[3,4-c]pyridin-2(3H)-yl)acetaldehyde

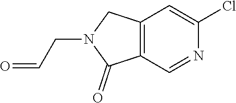 [SMILES: O=CCN1Cc2cc(Cl)ncc2C1=O]

To a mixture of 6-chloro-2-(2,2-dimethoxyethyl)-1H-pyrrolo[3,4-c]pyridin-3(2H)-one (0.4 g, 1.56 mmol) in CH₂Cl₂ (2 mL) was added trifluoroacetic acid (2 mL, 27.01 mmol) and water (0.08 mL, 4.44 mmol) in one portion. The mixture was stirred at rt for 2 h. The mixture was concentrated to afford 2-(6-chloro-3-oxo-1H-pyrrolo[3,4-c]pyridin-2(3H)-yl)acetaldehyde as a yellow gum (0.35 g, crude, m/z: 211 [M+H]⁺ observed). The material was used as is without any purification.

### (S)-6-Chloro-2-(2-(((5-oxopyrrolidin-2-yl)methyl)amino)ethyl)-1H-pyrrolo[3,4-c]pyridin-3(2H)-one

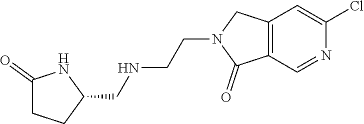 [SMILES: O=C1CC[C@@H](CNCCN2Cc3cc(Cl)ncc3C2=O)N1]

To a mixture of 2-(6-chloro-3-oxo-1H-pyrrolo[3,4-c]pyridin-2(3H)-yl)acetaldehyde (0.35 g, 1.66 mmol) and (S)-5-(aminomethyl)-2-pyrrolidinone, HCl (0.3 g, 1.99 mmol) in CH₂Cl₂/MeOH mixture (1:1, 4 mL) was added triethylamine (0.46 mL, 3.32 mmol) in one portion under N₂ atmosphere. The mixture was stirred at rt for 0.5 h. Sodium triacetoxyborohydride (0.7 g, 3.32 mmol) was added and the mixture was stirred at rt for 2.5 h. The solvent was evaporated to afford crude (S)-6-chloro-2-(2-(((5-oxopyrrolidin-2-yl)methyl)amino)ethyl)-1H-pyrrolo[3,4-c]pyridin-3(2H)-one (m/z: 309 [M+H]⁺ observed). The material was used for next step without purification.

### (S)-tert-Butyl (2-(6-chloro-3-oxo-1H-pyrrolo[3,4-c]pyridin-2(3H)-yl)ethyl)((5-oxopyrrolidin-2-yl)methyl)carbamate

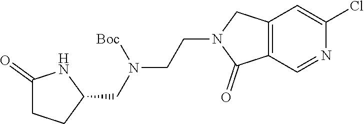 [SMILES: CC(C)(C)OC(=O)N(CCN1Cc2cc(Cl)ncc2C1=O)C[C@@H]1CCC(=O)N1]

To a mixture of (S)-6-chloro-2-(2-(((5-oxopyrrolidin-2-yl)methyl)amino)ethyl)-1H-pyrrolo[3,4-c]pyridin-3(2H)-one in a 5 mL of CH₂Cl₂/MeOH mixture was added di-tert-butyl dicarbonate (0.72 g, 3.32 mmol) in one portion under N₂ atmosphere. The mixture was stirred at rt for 0.5 h. The mixture was diluted with water (5 mL). The mixture was extracted with CH₂Cl₂ (2×10 mL). The combined organic phase was washed with brine (2×5 mL), dried with anhydrous sodium sulfate, filtered and concentrated. The residue was purified by normal phase SiO₂ chromatography (0-100% ethyl acetate/petroleum ether to 10% MeOH/ethyl acetate) to afford (S)-tert-butyl (2-(6-chloro-3-oxo-1H-pyrrolo[3,4-c]pyridin-2(3H)-yl)ethyl)((5-oxopyrrolidin-2-yl)methyl)carbamate as a yellow gum (0.32 g, 41% yield). ¹H NMR (400 MHz, Methanol-d₄): δ 8.64-8.58 (m, 1H), 7.63 (m, 1H), 4.59-4.53 (m, 2H), 3.89-3.79 (m, 1H), 3.68 (m, 2H), 3.54-3.41 (m, 2H), 3.31-3.25 (m, 2H), 2.28-2.16 (m, 3H), 1.75-1.71 (m, 1H), 1.23-1.11 (m, 9H).

### tert-Butyl N-[[6-[3-[3-[2-[2-[tert-butoxycarbonyl-[[(2S)-5-oxopyrrolidin-2-yl]methyl]amino]ethyl]-3-oxo-1H-pyrrolo[3,4-c]pyridin-6-yl]-2-chloro-phenyl]-2-chloro-phenyl]-2-methoxy-3-pyridyl]methyl]-N-[[(2S)-5-oxopyrrolidin-2-yl]methyl]carbamate

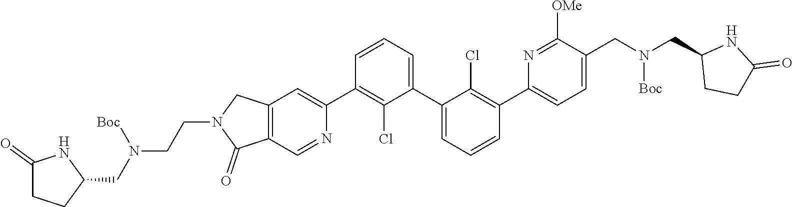 [SMILES: COc1nc(-c2cccc(-c3cccc(-c4cc5c(cn4)C(=O)N(CCN(C[C@@H]4CCC(=O)N4)C(=O)OC(C)(C)C)C5)c3Cl)c2Cl)ccc1CN(C[C@@H]1CCC(=O)N1)C(=O)OC(C)(C)C]

To a mixture of (S)-tert-butyl (2-(6-chloro-3-oxo-1H-pyrrolo[3,4-c]pyridin-2(3H)-yl)ethyl)((5-oxopyrrolidin-2-yl)methyl)carbamate (0.3 g, 0.73 mmol) and (S)-tert-butyl ((6-(2,2′-dichloro-3′-(4,4,5,5-tetramethyl-1,3,2-dioxaborolan-2-yl)-[1,1′-biphenyl]-3-yl)-2-methoxypyridin-3-yl)methyl)((5-oxopyrrolidin-2-yl)methyl)carbamate (0.75 g, 1.1 mmol) in dioxane/water mixture (6:1, 6.6 mL) was added [1,1′-bis(di-tert-butylphosphino)ferrocene]dichloropalladium(II) (0.02 g, 0.043 mmol) and potassium phosphate (0.47 g, 2.2 mmol) in one portion under N₂ atmosphere. The mixture was stirred at 110° C. for 0.5 h. The mixture was concentrated and the residue was purified by normal phase SiO₂ chromatography (0-100% (50% THE in ethyl acetate)/petroleum ether to 20% (50% THE in MeOH)/ethyl acetate) to afford tert-butyl N-[[6-[3-[3-[2-[2-[tert-butoxycarbonyl-[[(2S)-5-oxopyrrolidin-2-yl]methyl]amino]ethyl]-3-oxo-1H-pyrrolo[3,4-c]pyridin-6-yl]-2-chloro-phenyl]-2-chloro-phenyl]-2-methoxy-3-pyridyl]methyl]-N-[[(2S)-5-oxopyrrolidin-2-yl]methyl]carbamate as a yellow solid (0.32 g, 27% yield, m/z: 928 [M+H]⁺ observed).

### 6-(2,2′-Dichloro-3′-(6-methoxy-5-(((((S)-5-oxopyrrolidin-2-yl)methyl)amino)methyl)pyridin-2-yl)-[1,1′-biphenyl]-3-yl)-2-(2-((((S)-5-oxopyrrolidin-2-yl)methyl)amino)ethyl)-1H-pyrrolo[3,4-c]pyridin-3(2H)-one

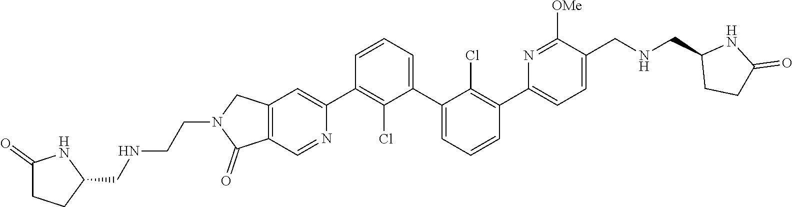 [SMILES: COc1nc(-c2cccc(-c3cccc(-c4cc5c(cn4)C(=O)N(CCNC[C@@H]4CCC(=O)N4)C5)c3Cl)c2Cl)ccc1CNC[C@@H]1CCC(=O)N1]

To a solution of tert-butyl N-[[6-[3-[3-[2-[2-[tert-butoxycarbonyl-[[(2S)-5-oxopyrrolidin-2-yl]methyl]amino]ethyl]-3-oxo-1H-pyrrolo[3,4-c]pyridin-6-yl]-2-chloro-phenyl]-2-chloro-phenyl]-2-methoxy-3-pyridyl]methyl]-N-[[(2S)-5-oxopyrrolidin-2-yl]methyl]carbamate (0.3 g, 0.32 mmol) in CH₂Cl₂ (1.5 mL) was added trifluoroacetic acid (3.2 mL, 43.4 mmol) in one portion. The mixture was stirred at rt for 1 h. The mixture was concentrated and the residue was purified by reverse phase HPLC to afford 6-(2,2′-dichloro-3′-(6-methoxy-5-(((((S)-5-oxopyrrolidin-2-yl)methyl)amino)methyl)pyridin-2-yl)-[1,1′-biphenyl]-3-yl)-2-(2-((((S)-5-oxopyrrolidin-2-yl)methyl)amino)ethyl)-1H-pyrrolo[3,4-c]pyridin-3(2H)-one as a yellow solid (9.5 mg, 3.8% yield, m/z: 728 [M+H]⁺ observed). ¹H NMR (400 MHz, DMSO-d₆): δ 8.97 (s, 1H), 7.92 (s, 1H), 7.81 (d, J=7.6 Hz, 1H), 7.67-7.51 (m, 7H), 7.45-7.43 (m, 1H), 7.26 (d, J=7.6 Hz, 1H), 4.67 (s, 2H), 3.91 (s, 3H), 3.75-3.70 (m, 2H), 3.61-3.53 (m, 4H), 2.80 (t, J=6 Hz, 2H), 2.55-2.53 (m, 2H), 2.13-2.00 (m, 6H), 1.69-1.60 (m, 2H).

### Example 483: 2-(2,2′-Dichloro-3′-(6-methoxy-5-(((((S)-5-oxopyrrolidin-2-yl)methyl)amino)methyl)pyridin-2-yl)-[1,1′-biphenyl]-3-yl)-6-(((((S)-5-oxopyrrolidin-2-yl)methyl)amino)methyl)isoindolin-1-one

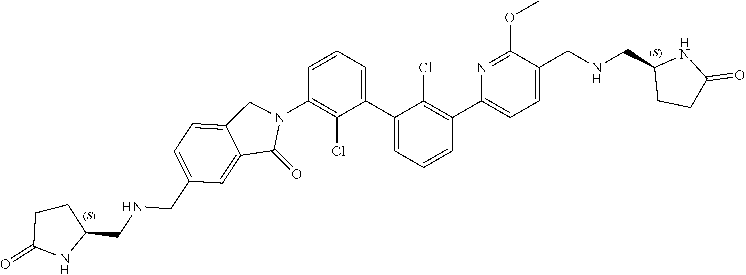

### Methyl 2-(bromomethyl)-5-iodobenzoate

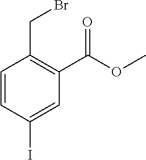

To a mixture of methyl 5-iodo-2-methylbenzoate (5 g, 18.11 mmol) and 1-bromopyrrolidine-2,5-dione (3.19 g, 17.93 mmol) in CHCl₃ (40 mL) was added AIBN (0.15 g, 0.91 mmol). The mixture was degassed and purged with N₂. The mixture was stirred at 75° C. for 12 h under N₂ atmosphere. The reaction mixture was diluted with water (40 ml) and the aqueous layer was extracted with EtOAc (3×30 mL). The combined organic layers were washed with brine (2×30 mL), dried over anhydrous sodium sulfate, filtered and concentrated under reduced pressure. The residue was purified by normal phase SiO₂ chromatography (0-100% EtOAc/petroleum ether) to afford methyl 2-(bromomethyl)-5-iodobenzoate as a yellow liquid (4.7 g, 71.65% yield). 1H NMR (400 MHz, DMSO-d₆) δ 8.12 (s, 1H), 7.92 (d, J=8.0 Hz, 1 H), 7.35 (d, J=8.0 Hz, 1H), 4.92 (s, 2H), 3.83 (s, 3H).

### Methyl 2-(((3-bromo-2-chlorophenyl)amino)methyl)-5-iodobenzoate

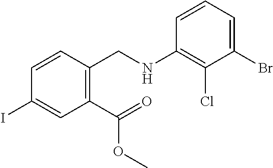 [SMILES: COC(=O)c1cc(I)ccc1CNc1cccc(Br)c1Cl]

To a mixture of methyl 2-(bromomethyl)-5-iodobenzoate (3.16 g, 8.89 mmol) and 3-bromo-2-chloroaniline (1.53 g, 7.41 mmol) in MeOH (15 mL) was added N-ethyl-N-propan-2-ylpropan-2-amine (2.58 mL). The mixture was stirred at 80° C. for 12 h under N₂. The reaction mixture was diluted with water (40 ml) and the aqueous layer was extracted with EtOAc (3×30 mL). The combined organic layers were washed with brine (2×30 mL), dried over anhydrous sodium sulfate, filtered and concentrated under reduced pressure. The residue was purified by normal phase SiO₂ chromatography chromatography (0-100% EtOAc/petroleum ether) to afford methyl 2-(((3-bromo-2-chlorophenyl)amino)methyl)-5-iodobenzoate as a white solid (2 g, 55% yield, m/z: 481.9 [M+H]⁺ observed). 2-(3-Bromo-2-chlorophenyl)-6-iodoisoindolin-1-one

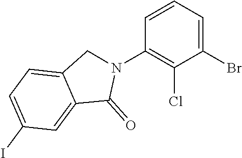 [SMILES: O=C1c2cc(I)ccc2CN1c1cccc(Br)c1Cl]

A mixture of methyl 2-(((3-bromo-2-chlorophenyl)amino)methyl)-5-iodobenzoate (2 g, 4.16 mmol) in glacial acetic acid (20 mL) was degassed and purged with N₂. The mixture was stirred at 140° C. for 2 h under N₂ atmosphere. The reaction mixture was concentrated under reduced pressure to give 2-(3-bromo-2-chlorophenyl)-6-iodoisoindolin-1-onen as a white solid (1.9 g, crude). ¹H NMR (400 MHz, DMSO-d₆) δ 8.08-8.07 (m, 1H), 8.01 (dd, J=1.6 Hz, J=6.4 Hz, 1H), 7.83 (dd, J=1.6 Hz, J=6.8 Hz, 1H), 7.62 (dd, J=1.6 Hz, J=6.8 Hz, 1H), 7.48 (d, J=7.6 Hz, 1H), 7.40 (t, J=8.4 Hz, 1H), 4.79 (s, 2H).

### 2-(3-Bromo-2-chlorophenyl)-6-vinylisoindolin-1-one

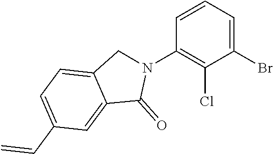 [SMILES: C=Cc1ccc2c(c1)C(=O)N(c1cccc(Br)c1Cl)C2]

To a mixture of 2-(3-bromo-2-chlorophenyl)-6-iodoisoindolin-1-one (1.4 g, 3.12 mmol), 4,4,5,5-tetramethyl-2-vinyl-1,3,2-dioxaborolane (1.32 mL) and sodium carbonate (1.32 g, 12.49 mmol) in THE (20 mL) and water (2 mL) was added tetrakis(triphenylphosphine)palladium (0.36 g, 0.31 mmol). The mixture was stirred at 80° C. for 6 h under N₂. The reaction mixture was diluted with water (40 ml) and the aqueous layer was extracted with EtOAc (3×30 mL). The combined organic layers were washed with brine (2×30 mL), dried over anhydrous sodium sulfate, filtered and concentrated under reduced pressure. The residue was purified by normal phase SiO₂ chromatography (0-100% EtOAc/petroleum ether) to afford 2-(3-bromo-2-chlorophenyl)-6-vinylisoindolin-1-one as a yellow solid (0.48 g, m/z: 350.0 [M+H]⁺ observed).

### 2-(3-Bromo-2-chlorophenyl)-3-oxoisoindoline-5-carbaldehyde

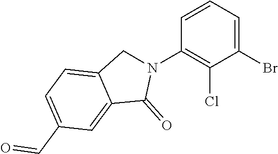 [SMILES: O=Cc1ccc2c(c1)C(=O)N(c1cccc(Br)c1Cl)C2]

To a mixture of 2-(3-bromo-2-chlorophenyl)-6-vinylisoindolin-1-one (0.30 g, 0.86 mmol) in THE (10 mL) and water (2 mL) at 0° C., sodium periodate(0.74 g, 3.44 mmol) and K₂OsO₄ (0.03 g, 0.17 mmol) were added. The mixture was stirred at 0° C. for 20 min and at rt for 4 h. The reaction mixture was diluted with water (40 ml) and the aqueous layer was extracted with EtOAc (3×30 mL). The combined organic layers were washed with brine (2×30 mL), dried over anhydrous sodium sulfate, filtered and concentrated under reduced pressure. The residue was purified by normal phase SiO₂ chromatography (0-100% EtOAc/petroleum ether) to afford 2-(3-bromo-2-chlorophenyl)-3-oxoisoindoline-5-carbaldehyde as a yellow solid (0.13 g). ¹H NMR (400 MHz, DMSO-d₆) δ 10.12 (s, 1H), 8.30 (s, 1H), 8.19 (dd, J=1.2 Hz, J=6.8 Hz, 1H), 7.88-7.84 (m, 2H), 7.66 (dd, J=1.6 Hz, J=6.4 Hz, 1H), 7.41 (t, J=8.0 Hz, 1 H), 4.97 (s, 2H).

### 2-(2,2′-Dichloro-3′-(5-formyl-6-methoxypyridin-2-yl)-[1,1′-biphenyl]-3-yl)-3-oxoisoindoline-5-carbaldehyde

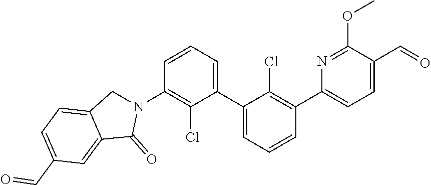 [SMILES: COc1nc(-c2cccc(-c3cccc(N4Cc5ccc(C=O)cc5C4=O)c3Cl)c2Cl)ccc1C=O]

To a mixture of 2-(3-bromo-2-chlorophenyl)-3-oxoisoindoline-5-carbaldehyde (0.04 g, 0.11 mmol,), 6-(2-chloro-3-(4,4,5,5-tetramethyl-1,3,2-dioxaborolan-2-yl)phenyl)-2-methoxynicotinaldehyde (0.6 g, 0.17 mmol) and potassium phosphate (0.06 g, 0.29 mmol) in dioxane (4 mL) and H₂O (0.4 mL) was added [1,1′-bis(di-tert-butylphosphino)ferrocene]dichloropalladium(II) (0.007 g, 0.01 mmol,). The mixture was stirred at 110° C. for 0.5 h under N₂. The reaction mixture was diluted with water (40 ml) and the aqueous layer was extracted with EtOAc (3×30 mL). The combined organic layers were washed with brine (2×30 mL), dried over anhydrous sodium sulfate, filtered and concentrated under reduced pressure. The residue was purified by normal phase SiO₂ chromatography (0-100% EtOAc/petroleum ether) to afford 2-(2,2′-dichloro-3′-(5-formyl-6-methoxypyridin-2-yl)-[1,1′-biphenyl]-3-yl)-3-oxoisoindoline-5-carbaldehyde as a yellow solid (0.09 g, m/z: 517.3 [M+H]⁺ observed).

### 2-(2,2′-Dichloro-3′-(6-methoxy-5-(((((S)-5-oxopyrrolidin-2-yl)methyl)amino)methyl)pyridin-2-yl)-[1,1′-biphenyl]-3-yl)-6-(((((S)-5-oxopyrrolidin-2-yl)methyl)amino)methyl)isoindolin-1-one

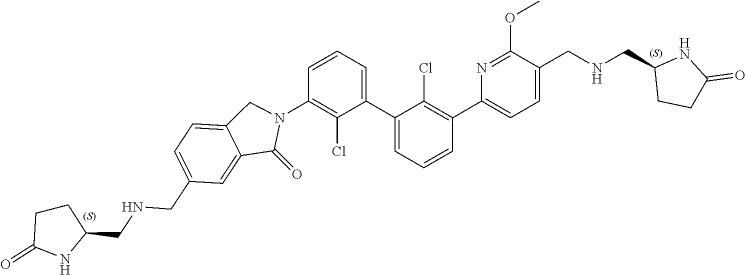

A mixture of 2-(2,2′-dichloro-3′-(5-formyl-6-methoxypyridin-2-yl)-[1,1′-biphenyl]-3-yl)-3-oxoisoindoline-5-carbaldehyde (0.08 g, 0.16 mmol), (S)-5-(aminomethyl)pyrrolidin-2-one, HCl (0.09 g, 0.58 mmol), sodium acetate (0.04 g, 0.49 mmol) and 4A molecular sieve (0.05 g, 0.16 mmol) in DCM (8 mL) was degassed and purged with N₂. The mixture was stirred at rt for 11.5 h under N₂ atmosphere. Sodium cyanoborohydride (0.03 g, 0.49 mmol) was added and the mixture was stirred at rt for 0.5 h under N₂ atmosphere. The reaction mixture was concentrated and the residue was purified by reverse phase HPLC to afford 2-(2,2′-dichloro-3′-(6-methoxy-5-(((((S)-5-oxopyrrolidin-2-yl)methyl)amino)methyl)pyridin-2-yl)-[1,1′-biphenyl]-3-yl)-6-(((((S)-5-oxopyrrolidin-2-yl)methyl)amino)methyl)isoindolin-1-one as a white solid (18.7 m g, m/z: 713.3 [M+H]⁺ observed). ¹H NMR (400 MHz, DMSO-d₆) δ 7.75-7.71 (m, 2H), 7.60-7.57 (m, 5H), 7.54-7.51 (m, 1H), 7.49-7.44 (m, 2H), 7.39 (dd, J=1.6 Hz, J=6.4 Hz, 1H), 7.34 (dd, J=1.6 Hz, J=6.0 Hz, 1H), 7.18 (d, J=7.2 Hz, 1H), 4.78 (s, 2H), 3.83 (s, 3H), 3.78-3.77 (m, 2H), 3.65 (m, 2H), 3.57-3.54 (m, 3H), 2.50-2.49 (m, 2H), 2.45 (m, 2H), 2.08-1.95 (m, 6H) 1.66-1.55 (m, 2H).

### Example 506: (5S)-5-[[[6-[2-Chloro-3-[2-chloro-3-[2-[[[(2S)-5-oxopyrrolidin-2-yl]methylamino]methyl]-[1,2,4]triazolo[1,5-a]pyrimidin-6-yl]phenyl]phenyl]-2-methoxy-3-pyridyl]methylamino] methyl]pyrrolidin-2-one

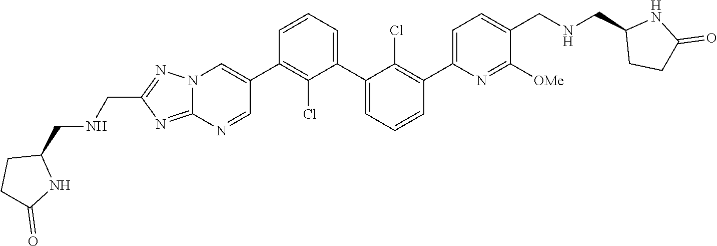 [SMILES: COc1nc(-c2cccc(-c3cccc(-c4cnc5nc(CNC[C@@H]6CCC(=O)N6)nn5c4)c3Cl)c2Cl)ccc1CNC[C@@H]1CCC(=O)N1]

### (6-bromo-[1,2,4]triazolo[1,5-a]pyrimidin-2-yl)methanol

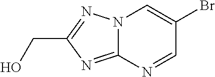 [SMILES: OCc1nc2ncc(Br)cn2n1]

A mixture of (5-amino-4H-1,2,4-triazol-3-yl)methanol (200 mg, 1.75 mmol) and 2-bromopropanedial (264.59 mg, 1.75 mmol) in acetic acid (2 mL) was heated to 60° C. for 15 h. The solvent was evaporated and the residue was purified by silica gel chromatography (5% MeOH in DCM) to afford (6-bromo-[1,2,4]triazolo[1,5-a]pyrimidin-2-yl)methanol (135 mg, 33.6%). m/z: 230.9 [M+H]⁺ observed. ¹H NMR (400 MHz, Methanol-d₄) δ 10.06-10.00 (m, 1H), 9.46-9.40 (m, 1H), 5.36-5.30 (m, 2H).

### tert-Butyl N-[[6-[2-chloro-3-[2-chloro-3-[2-(hydroxymethyl)-[1,2,4]triazolo[1,5-a]pyrimidin-6-yl]phenyl]phenyl]-2-methoxy-3-pyridyl]methyl]-N-[[(2S)-5-oxopyrrolidin-2-yl]methyl]carbamate

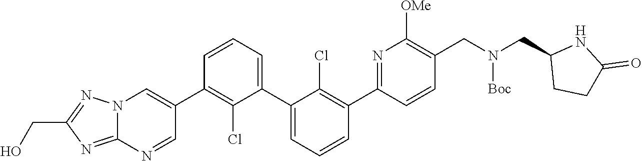 [SMILES: COc1nc(-c2cccc(-c3cccc(-c4cnc5nc(CO)nn5c4)c3Cl)c2Cl)ccc1CN(C[C@@H]1CCC(=O)N1)C(=O)OC(C)(C)C]

To a mixture of tert-butyl N-[[6-[2-chloro-3-[2-chloro-3-(4,4,5,5-tetramethyl-1,3,2-dioxaborolan-2-yl)phenyl]phenyl]-2-methoxy-3-pyridyl]methyl]-N-[[(2S)-5-oxopyrrolidin-2-yl]methyl]carbamate (442 mg, 0.65 mmol) and (6-bromo-[1,2,4]triazolo[1,5-a]pyrimidin-2-yl)methanol (135 mg, 0.59 mmol) in dioxane/water (4:1, 3 mL), potassium carbonate (70 mg, 0.51 mmol) was added. Nitrogen gas was bubbled through the mixture for two minutes. Tetrakis(triphenylphosphine)palladium(0) (39 mg, 0.03 mmol) was added and the reaction was stirred at 100° C. for 0.5 h in a microwave. The reaction mixture was diluted with water (30 mL), and the aqueous layer was extracted with EtOAc (3×30 ml). The combined organic layers were washed with brine (20 mL), dried over anhydrous sodium sulfate, filtered and concentrated. The residue was purified by normal phase silica gel chromatography (0-5% MeOH in DCM) to afford tert-butyl N-[[6-[2-chloro-3-[2-chloro-3-[2-(hydroxymethyl)-[1,2,4]triazolo[1,5-a]pyrimidin-6-yl]phenyl]phenyl]-2-methoxy-3-pyridyl]methyl]-N-[[(2S)-5-oxopyrrolidin-2-yl]methyl]carbamate (47 mg, 11.3%). m/z: 704.2 [M+H]⁺ observed.

### tert-Butyl N-[[6-[2-chloro-3-[2-chloro-3-[2-[[[(2S)-5-oxopyrrolidin-2-yl]methylamino]methyl]-[1,2,4]triazolo[1,5-a]pyrimidin-6-yl]phenyl]phenyl]-2-methoxy-3-pyridyl]methyl]-N-[[(2S)-5-oxopyrrolidin-2-yl]methyl]carbamate

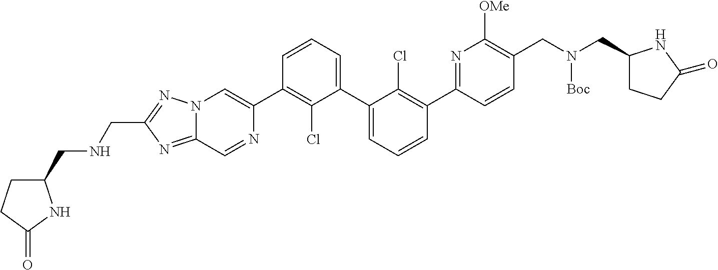 [SMILES: COc1nc(-c2cccc(-c3cccc(-c4cn5nc(CNC[C@@H]6CCC(=O)N6)nc5cn4)c3Cl)c2Cl)ccc1CN(C[C@@H]1CCC(=O)N1)C(=O)OC(C)(C)C]

To a solution of tert-butyl N-[[6-[2-chloro-3-[2-chloro-3-[2-(hydroxymethyl)-[1,2,4]triazolo[1,5-a]pyrimidin-6-yl]phenyl]phenyl]-2-methoxy-3-pyridyl]methyl]-N-[[(2S)-5-oxopyrrolidin-2-yl]methyl]carbamate (23 mg, 0.03 mmol) in DCM (0.4 mL) was added mesyl chloride (3.79 uL, 0.05 mmol) and triethylamine (13.65 uL, 0.10 mmol). The reaction was stirred at rt for 1 h. The solvent was evaporated, and the residue was taken up in DMF (0.4 mL). Potassium carbonate (14 mg, 0.10 mmol) and (5S)-5-(aminomethyl)pyrrolidin-2-one (11 mg, 0.10 mmol) were added and the reaction was stirred at rt for 12 h. LCMS suggested formation of the desired product. The solvent was evaporated, and the residue was suspended in water. The precipitate formed was collected to afford tert-butyl N-[[6-[2-chloro-3-[2-chloro-3-[2-[[[(2S)-5-oxopyrrolidin-2-yl]methylamino]methyl]-[1,2,4]triazolo[1,5-a]pyrimidin-6-yl]phenyl]phenyl]-2-methoxy-3-pyridyl]methyl]-N-[[(2S)-5-oxopyrrolidin-2-yl]methyl]carbamate (17 mg, 65.0%). The material was used as is without any purification. m/z: 802.1 [M+H]⁺ observed.

### (5S)-5-[[[6-[2-Chloro-3-[2-chloro-3-[2-[[[(2S)-5-oxopyrrolidin-2-yl]methylamino]methyl]-[1,2,4]triazolo[1,5-a]pyrimidin-6-yl]phenyl]phenyl]-2-methoxy-3-pyridyl]methylamino] methyl]pyrrolidin-2-one

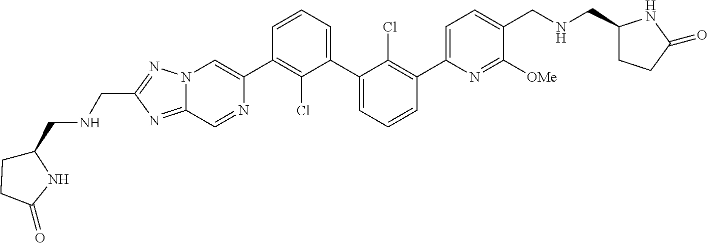 [SMILES: COc1nc(-c2cccc(-c3cccc(-c4cn5nc(CNC[C@@H]6CCC(=O)N6)nc5cn4)c3Cl)c2Cl)ccc1CNC[C@@H]1CCC(=O)N1]

To a solution of tert-butyl N-[[6-[2-chloro-3-[2-chloro-3-[2-[[[(2S)-5-oxopyrrolidin-2-yl]methylamino]methyl]-[1,2,4]triazolo[1,5-a]pyrimidin-6-yl]phenyl]phenyl]-2-methoxy-3-pyridyl]methyl]-N-[[(2S)-5-oxopyrrolidin-2-yl]methyl]carbamate (9 mg, 0.01 mmol) in DCM (0.1 mL) was added TFA (16 uL, 0.21 mmol). The reaction was stirred at rt for 1 h. The solvent was evaporated and the residue was purified by reverse phase HPLC (acetonitrile:water) to afford (5S)-5-[[[6-[2-chloro-3-[2-chloro-3-[2-[[[(2S)-5-oxopyrrolidin-2-yl]methylamino] methyl]-[1,2,4]triazolo[1,5-a]pyrimidin-6-yl]phenyl]phenyl]-2-methoxy-3-pyridyl]methylamino] methyl]pyrrolidin-2-one (4.8 mg, 64.5%). m/z: 701.4 [M+H]⁺ observed. ¹H NMR (400 MHz, CD₃OD): δ9.37 (d, J=2.3 Hz, 1H), 9.00 (d, J=2.4 Hz, 1H), 7.74 (d, J=7.5 Hz, 1H), 7.68-7.63 (m, 2H), 7.59 (t, J=7.6 Hz, 1H), 7.51 (ddd, J=7.7, 4.8, 3.0 Hz, 2H), 7.39 (dd, J=7.6, 1.8 Hz, 1H), 7.25 (d, J=7.4 Hz, 1H), 4.12 (d, J=3.1 Hz, 2H), 4.02 (s, 3H), 3.88 (d, J=3.9 Hz, 2H), 3.84 (t, J=6.8 Hz, 2H), 2.84 (dd, J=12.1, 4.9 Hz, 1H), 2.79-2.71 (m, 3H), 2.37-2.22 (m, 6H), 1.91-1.76 (m, 2H).

### Example 331: 6-(2,2′-Dichloro-3′-(6-methoxy-5-(((((S)-5-oxopyrrolidin-2-yl)methyl)amino)methyl)pyridin-2-yl)-[1,1′-biphenyl]-3-yl)-3-methyl-2-(((((S)-5-oxopyrrolidin-2-yl)methyl)amino) methyl)pyrrolo[2,1-f][1,2,4]triazin-4(3H)-one

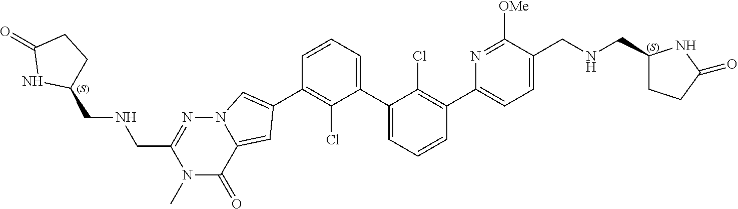

### 1-(4-Bromo-1H-pyrrol-2-yl)-2,2,2-trichloroethanone

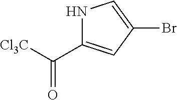

To a cooled solution (0° C.) of 2,2,2-trichloro-1-(1H-pyrrol-2-yl)ethanone (20 g, 94 mmol) in acetonitrile (100 mL) was added NBS (16.8 g, 94 mmol) in portions. The reaction was stirred at 25° C. for 1.5 h. The reaction was quenched by the slow addition of water (15 mL, 15.0 mL/g) and cooled to 0° C. The solids were allowed to desaturate for an additional 1 h. The solids were filtered, washed with water (15 mL, 5.0 mL/g), and dried in vacuo to 1-(4-bromo-1H-pyrrol-2-yl)-2,2,2-trichloroethanone as a black solid (25 g, 91% yield). ¹H NMR (400 MHz, DMSO-d₆) δ=12.84 (s, 1H), 7.54 (dd, J=1.2 Hz, J=3.2 Hz, 1H), 7.33 (dd, J=1.2 Hz, J=2.4 Hz, 1H)

### 4-Bromo-1H-pyrrole-2-carboxamide

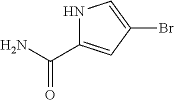

To the mixture of 1-(4-bromo-1H-pyrrol-2-yl)-2,2,2-trichloroethanone (25 g, 85.8 mmol) in acetonitrile (125 mL) was added 30% of NH₃.H₂O (75 mL). The reaction was stirred at 25° C. for 12 h. The reaction mixture was concentrated under reduced pressure to afford 4-bromo-1H-pyrrole-2-carboxamide as a black solid (15 g, 92% yield).

### 1-Amino-4-bromo-1H-pyrrole-2-carboxamide

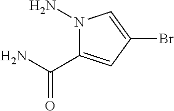

4-Bromo-1H-pyrrole-2-carboxamide (5 g, 26.4 mmol) was slowly added to a suspension of NaH (1.59 g, 39.7 mmol) in DMF (15 mL) at 0° C. After stirring at 0° C. for 1 hour, a solution of O-(2,4-dinitrophenyl)hydroxylamine (8.43 g, 42.3 mmol) in DMF (3 mL) was added dropwise over the period of 30 minutes. The resulting reaction mixture was stirred at 0° C. for 1.5 h. The reaction was quenched by slow addition of saturated sodium thiosulfate solution (120 mL). The resulting mixture was extracted with ethyl acetate (3×80 mL). The organic layer was washed with 10% lithium chloride aqueous solution (3×60 mL), dried with sodium sulfate, and filtered. The filtrate was concentrated, and the residue was purified by normal phase SiO₂ chromatography (0-100% EtOAc/petroleum ether) to afford crude product. The crude product was triturated with 10% lithium chloride aqueous solution (20 mL) at 25° C. for 30 min to get 1-amino-4-bromo-1H-pyrrole-2-carboxamide (3 g, 55% yield). ¹H NMR (400 MHz, DMSO-d₆) δ=7.93 (br, 1H), 7.27-7.25 (m, 1H), 7.00 (d, J=2.4 Hz, 1H), 6.74 (d, J=2.4 Hz, 1H)

### Ethyl 2-((4-bromo-2-carbamoyl-1H-pyrrol-1-yl)amino)-2-oxoacetate

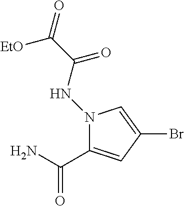 [SMILES: CCOC(=O)C(=O)Nn1cc(Br)cc1C(N)=O]

To a mixture of 1-amino-4-bromo-1H-pyrrole-2-carboxamide (3 g, 12.5 mmol) and pyridine (0.99 g, 12.5 mmol) in THF (10 mL) was added ethyl 2-chloro-2-oxoacetate (1.71 g, 12.5 mmol). The resulting reaction mixture was stirred at 25° C. for 3 h. The mixture was concentrated under reduce pressure. DCM (10 mL) was added, and the suspended solid was collected by filtration to afford ethyl 2-((4-bromo-2-carbamoyl-1H-pyrrol-1-yl)amino)-2-oxoacetate as a yellow solid (2.5 g, 65% yield). ¹H NMR (400 MHz, DMSO-d₆): δ 12.16 (s, 1H), 7.59 (br, 1H), 7.20 (d, J=2.0 Hz, 1H), 7.08 (br, 1H), 6.94 (d, J=2.0 Hz, 1H), 4.31 (q, J=7.2 Hz, 2H), 1.31 (t, J=7.2 Hz, 3H)

### Ethyl 6-bromo-4-oxo-3,4-dihydropyrrolo[2,1-f][1,2,4]triazine-2-carboxylate

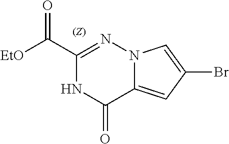 [SMILES: CCOC(=O)c1nn2cc(Br)cc2c(=O)[nH]1]

A mixture of ethyl 2-((4-bromo-2-carbamoyl-1H-pyrrol-1-yl)amino)-2-oxoacetate (2.1 g, 6.93 mmol), chlorotrimethylsilane (16.6 g, 151.8 mmol) and triethylamine (21.2 mL, 151.8 mmol) in 1,2-dichloroethane (250 mL) was heated at 110° C. for 1 h in a microwave. The reaction mixture was filtered, and the filtrate was concentrated. The reaction mixture was diluted with water (80 mL) and extracted with EtOAc (3×60 ml). The combined organic layers were washed with brine (50 mL), dried over anhydrous sodium sulfate, filtered and concentrated under reduced pressure. The residue was purified by normal phase SiO₂ chromatography (0-100% EtOAc/petroleum ether) to afford ethyl 6-bromo-4-oxo-3,4-dihydropyrrolo[2,1-f][1,2,4]triazine-2-carboxylate as a yellow solid (1.1 g). ¹H NMR (400 MHz, DMSO-d₆): δ 12.31 (br, 1H), 8.05 (s, 1H), 7.13 (s, 1H), 4.39 (q, J=7.2 Hz, 2H), 1.35 (t, J=7.2 Hz, 3H)

### Ethyl 6-bromo-3-methyl-4-oxo-3,4-dihydropyrrolo[2,1-f][1,2,4]triazine-2-carboxylate

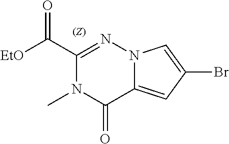 [SMILES: CCOC(=O)c1nn2cc(Br)cc2c(=O)n1C]

To a mixture of ethyl 6-bromo-4-oxo-3,4-dihydropyrrolo[2,1-f][1,2,4]triazine-2-carboxylate (1.5 g, 5.24 mmol) and potassium carbonate (0.72 g, 5.24 mmol) in DMF (20 mL), iodomethane (0.74 g, 5.24 mmol) was added dropwise. The mixture was stirred at 25° C. for 1 h. The reaction mixture was quenched with water (30 mL) and saturated ammonium chloride (50 mL) solution. The aqueous layer was extracted with EtOAc (3×20 mL). The combined organic layers were washed with brine (20 mL), dried over anhydrous sodium sulfate, filtered and concentrated under reduced pressure. The residue was purified by normal phase SiO₂ chromatography (0-100% EtOAc/petroleum ether) to afford ethyl 6-bromo-3-methyl-4-oxo-3,4-dihydropyrrolo[2,1-f][1,2,4]triazine-2-carboxylate as a yellow solid (1.02 g, 64% yield). ¹H NMR (400 MHz, DMSO-d₆): δ 7.95 (d, J=2.4 Hz, 1H), 7.09 (d, J=2 Hz, 1H), 4.40 (q, J=7.6 Hz, 2H), 3.42 (s, 3H), 1.32 (t, J=7.6 Hz, 3H)

### 6-Bromo-2-(hydroxymethyl)-3-methylpyrrolo[2,1-f][1,2,4]triazin-4(3H)-one

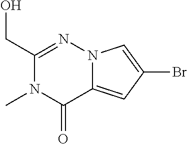

To a mixture of ethyl 6-bromo-3-methyl-4-oxo-3,4-dihydropyrrolo[2,1-f][1,2,4]triazine-2-carboxylate (0.5 g, 1.67 mmol) in THF/MeOH (10:11, 21 mL) at 0° C., sodium borohydride (0.19 g, 5 mmol) was added. The mixture was stirred at 20° C. for 2 hours. The reaction was quenched by addition of water (20 mL) and extracted with EtOAc (3×30 ml). The combined organic layers were washed with brine (20 mL), dried over anhydrous sodium sulfate, filtered and concentrated under reduced pressure. The residue was purified by SiO₂ chromatography (0-100% EtOAc/petroleum ether) to afford 6-bromo-2-(hydroxymethyl)-3-methylpyrrolo[2,1-f][1,2,4]triazin-4(3H)-one as a yellow solid (0.35 g, 81% yield)¹H NMR (400 MHz, DMSO-d₆): δ7.83 (d, J=2.0 Hz, 1H), 7.00 (d, J=2 Hz, 1H), 5.88 (t, J=7 Hz, 1H), 4.48 (d, J=5.6 Hz, 2H), 3.50 (s, 3H).

### Example 423: (S)-tert-Butyl ((6-(2,2′-dichloro-3′-(2-(hydroxymethyl)-3-methyl-4-oxo-3,4-dihydropyrrolo [2,1-f][1,2,4]triazin-6-yl)-[1,1′-biphenyl]-3-yl)-2-methoxypyridin-3-yl)methyl)((5-oxopyrrolidin-2-yl)methyl)carbamate

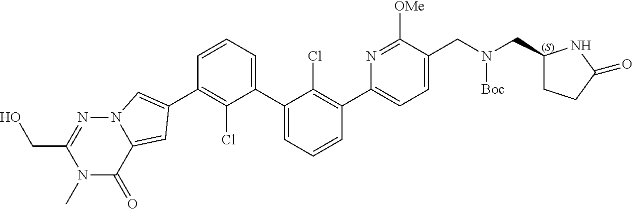

To a mixture of 6-bromo-2-(hydroxymethyl)-3-methylpyrrolo[2,1-f][1,2,4]triazin-4(3H)-one (0.35 g, 1.36 mmol) in dioxane/water (10:1, 8.8 mL), potassium phosphate (0.86 g, 4.07 mmol), (S)-tert-butyl ((6-(2,2′-dichloro-3′-(4,4,5,5-tetramethyl-1,3,2-dioxaborolan-2-yl)-[1,1′-biphenyl]-3-yl)-2-methoxypyridin-3-yl)methyl)((5-oxopyrrolidin-2-yl)methyl)carbamate (1.02 g, 1.49 mmol) and [1,1′-bis(di-tert-butylphosphino)ferrocene]dichloropalladium(II) (0.09 g, 0.1 eq) were added. The mixture was stirred at 110° C. for 0.5 h. The reaction mixture was diluted with water (30 mL), and the aqueous layer was extracted with EtOAc (3×30 ml). The combined organic layers were washed with brine (20 mL), dried over anhydrous sodium sulfate, filtered and concentrated under reduced pressure. The residue was purified by normal phase SiO₂ chromatography (0-100% EtOAc/petroleum ether) to afford (S)-tert-butyl ((6-(2,2′-dichloro-3′-(2-(hydroxymethyl)-3-methyl-4-oxo-3,4-dihydropyrrolo[2,1-f][1,2,4]triazin-6-yl)-[1,1′-biphenyl]-3-yl)-2-methoxypyridin-3-yl)methyl)((5-oxopyrrolidin-2-yl)methyl)carbamate as a yellow solid (0.8 g, 80% yield, m/z: 733.2 [M+H]⁺ observed).

### (S)-tert-Butyl ((6-(2,2′-dichloro-3′-(2-formyl-3-methyl-4-oxo-3,4-dihydropyrrolo[2,1-f][1,2,4]triazin-6-yl)-[1,1′-biphenyl]-3-yl)-2-methoxypyridin-3-yl)methyl)((5-oxopyrrolidin-2-yl)methyl)carbamate

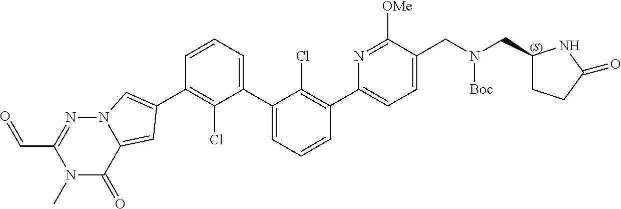

To a mixture (S)-tert-butyl ((6-(2,2′-dichloro-3′-(2-(hydroxymethyl)-3-methyl-4-oxo-3,4-dihydropyrrolo[2,1-f][1,2,4]triazin-6-yl)-[1,1′-biphenyl]-3-yl)-2-methoxypyridin-3-yl)methyl)((5-oxopyrrolidin-2-yl)methyl)carbamate (0.6 g, 0.82 mmol) in DCM (8 mL), Dess-Martin (0.69 g, 1.64 mmol) was added. The mixture was stirred at 25° C. for 2 h. The reaction mixture was filtered, and the filtrate was concentrated. The residue was purified by normal phase SiO₂ chromatography (0-100% EtOAc/petroleum ether) to afford (S)-tert-butyl ((6-(2,2′-dichloro-3′-(2-formyl-3-methyl-4-oxo-3,4-dihydropyrrolo[2,1-f][1,2,4]triazin-6-yl)-[1,1′-biphenyl]-3-yl)-2-methoxypyridin-3-yl)methyl)((5-oxopyrrolidin-2-yl)methyl)carbamate as a yellow solid (0.29 g, 48% yield, m/z: 731.2[M+H]⁺).

### tert-Butyl ((6-(2,2′-dichloro-3′-(3-methyl-4-oxo-2-(((((S)-5-oxopyrrolidin-2-yl)methyl) amino)methyl)-3,4-dihydropyrrolo[2,1-f][1,2,4]triazin-6-yl)-[1,1′-biphenyl]-3-yl)-2-methoxypyridin-3-yl)methyl)(((S)-5-oxopyrrolidin-2-yl)methyl)carbamate

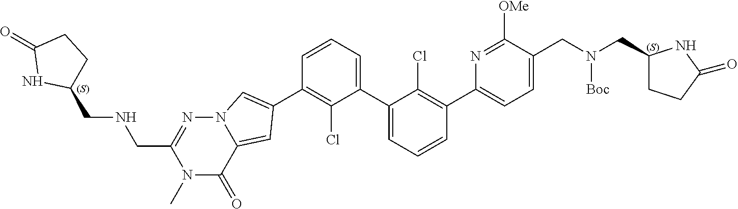

To a mixture(S)-tert-butyl ((6-(2,2′-dichloro-3′-(2-formyl-3-methyl-4-oxo-3,4-dihydropyrrolo [2,1-f][1,2,4]triazin-6-yl)-[1,1′-biphenyl]-3-yl)-2-methoxypyridin-3-yl)methyl)((5-oxopyrrolidin-2-yl)methyl)carbamate (0.15 g, 0.21 mmol) in DCM (5 mL) was added sodium acetate (0.05 g, 0.62 mmol) and (S)-5-(aminomethyl)pyrrolidin-2-one, HCl (0.06 g, 0.41 mmol). The mixture was stirred at 25° C. for 11 h. Sodium cyanoborohydride (0.03 g, 0.41 mmol) was added and the stirring continued for 1 h. The reaction mixture was concentrated to give tert-butyl ((6-(2,2′-dichloro-3′-(3-methyl-4-oxo-2-(((((S)-5-oxopyrrolidin-2-yl)methyl)amino)methyl)-3,4-dihydropyrrolo[2,1-f][1,2,4]triazin-6-yl)-[1,1′-biphenyl]-3-yl)-2-methoxypyridin-3-yl)methyl)(((S)-5-oxopyrrolidin-2-yl)methyl)carbamate as a yellow solid (0.2 g, crude, m/z: 829.2 [M+H]⁺ observed).

### 6-(2,2′-Dichloro-3′-(6-methoxy-5-(((((S)-5-oxopyrrolidin-2-yl)methyl)amino)methyl)pyridin-2-yl)-[1,1′-biphenyl]-3-yl)-3-methyl-2-(((((S)-5-oxopyrrolidin-2-yl)methyl)amino) methyl)pyrrolo[2,1-f][1,2,4]triazin-4(3H)-one

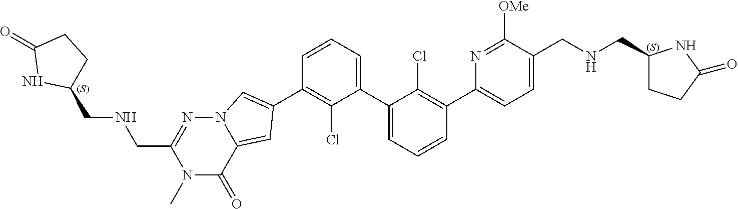

To a mixture of tert-butyl ((6-(2,2′-dichloro-3′-(3-methyl-4-oxo-2-(((((S)-5-oxopyrrolidin-2-yl)methyl)amino)methyl)-3,4-dihydropyrrolo[2,1-f][1,2,4]triazin-6-yl)-[1,1′-biphenyl]-3-yl)-2-methoxypyridin-3-yl)methyl)(((S)-5-oxopyrrolidin-2-yl)methyl)carbamate (0.2 g, 0.24 mmol) in DCM (1 mL), trifluoroacetic acid (3 mL) was added. The mixture was stirred at rt for 10 min. The reaction mixture was concentrated under reduced pressure. The residue was purified by reverse phase HPLC to afford 6-(2,2′-dichloro-3′-(6-methoxy-5-(((((S)-5-oxopyrrolidin-2-yl)methyl)amino)methyl)pyridin-2-yl)-[1,1′-biphenyl]-3-yl)-3-methyl-2-(((((S)-5-oxopyrrolidin-2-yl)methyl)amino)methyl)pyrrolo[2,1-f][1,2,4]triazin-4(3H)-one as a white solid (31.5 mg, 17% yield, m/z: 729.3 [M+H]⁺ observed)¹H NMR (400 MHz, Methanol-d₄): δ 7.95 (d, J=1.6 Hz, 1H), 7.91 (d, J=7.6 Hz, 1H), 7.68-7.65 (m, 2H), 7.53-7.49 (m, 2H), 7.42-7.36 (m, 3H), 7.32-7.29 (m, 1H), 4.62-4.52 (m, 2H), 4.40-4.33 (m, 2H), 4.17-4.06 (m, 5H), 3.58-3.54 (m, 3H), 3.39-3.35 (m, 2H), 3.30-3.27 (m, 2H), 2.50-2.38 (m, 6H), 2.00-1.88 (m, 2H).

### Example 350: 6-(2,2′-Dichloro-3′-(6-methoxy-5-(((((S)-5-oxopyrrolidin-2-yl)methyl)amino)methyl)pyridin-2-yl)-[1,1′-biphenyl]-3-yl)-2-((((S)-2-hydroxypropyl)amino)methyl)-3-methylpyrrolo[2,1-f][1,2,4]triazin-4(3H)-one

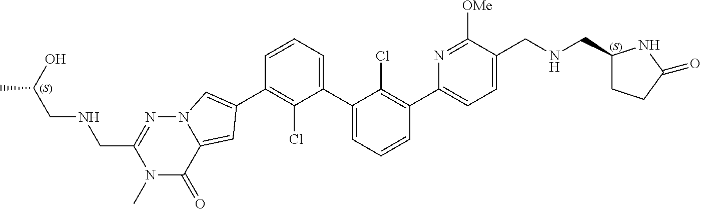

### tert-Butyl ((6-(2,2′-dichloro-3′-(2-((((S)-2-hydroxypropyl)amino)methyl)-3-methyl-4-oxo-3,4-dihydropyrrolo[2,1-f][1,2,4]triazin-6-yl)-[1,1′-biphenyl]-3-yl)-2-methoxypyridin-3-yl)methyl)(((S)-5-oxopyrrolidin-2-yl)methyl)carbamate

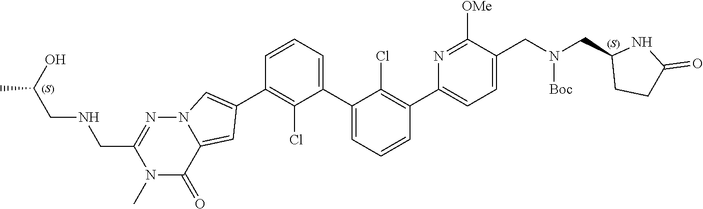

To a mixture (S)-tert-butyl ((6-(2,2′-dichloro-3′-(2-formyl-3-methyl-4-oxo-3,4-dihydropyrrolo [2,1-f][1,2,4]triazin-6-yl)-[1,1′-biphenyl]-3-yl)-2-methoxypyridin-3-yl)methyl)((5-oxopyrrolidin-2-yl)methyl)carbamate (0.16 g, 0.22 mmol) in DCM (2 mL) was added sodium acetate (0.05 g, 0.66 mmol) and (S)-1-aminopropan-2-ol (0.03 g, 0.44 mmol). The mixture was stirred at 25° C. for 11 h. Sodium cyanoborohydride (0.03 g, 0.44 mmol) was added. The mixture was stirred at 25° C. for 1 h. The reaction mixture was concentrated under reduced pressure to give tert-butyl ((6-(2,2′-dichloro-3′-(2-((((S)-2-hydroxypropyl)amino)methyl)-3-methyl-4-oxo-3,4-dihydropyrrolo[2,1-f][1,2,4]triazin-6-yl)-[1,1′-biphenyl]-3-yl)-2-methoxypyridin-3-yl)methyl) (((S)-5-oxopyrrolidin-2-yl)methyl)carbamate as a yellow solid (0.2 g, crude), m/z: 790.4 [M+H]⁺ observed).

### 6-(2,2′-Dichloro-3′-(6-methoxy-5-(((((S)-5-oxopyrrolidin-2-yl)methyl)amino)methyl)pyridin-2-yl)-[1,1′-biphenyl]-3-yl)-2-((((S)-2-hydroxypropyl)amino)methyl)-3-methylpyrrolo[2,1-f][1,2,4]triazin-4(3H)-one

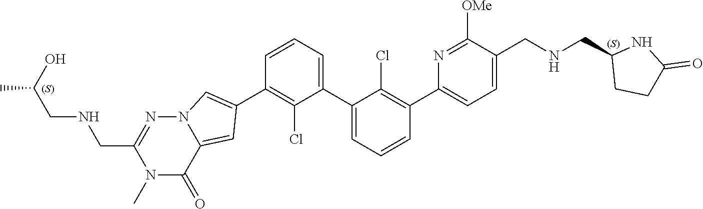

To a mixture tert-butyl ((6-(2,2′-dichloro-3′-(2-((((S)-2-hydroxypropyl)amino)methyl)-3-methyl-4-oxo-3,4-dihydropyrrolo[2,1-f][1,2,4]triazin-6-yl)-[1,1′-biphenyl]-3-yl)-2-methoxypyridin-3-yl)methyl)(((S)-5-oxopyrrolidin-2-yl)methyl)carbamate (0.2 g, 0.25 mmol) in DCM (3 mL) was added TFA (3.00 mL). The mixture was stirred at 25° C. for 10 min. The reaction mixture was concentrated under reduced pressure to give a residue. The residue was purified by reverse phase HPLC to 6-(2,2′-dichloro-3′-(6-methoxy-5-(((((S)-5-oxopyrrolidin-2-yl)methyl)amino) methyl)pyridin-2-yl)-[1,1′-biphenyl]-3-yl)-2-((((S)-2-hydroxypropyl)amino) methyl)-3-methylpyrrolo[2,1-f][1,2,4]triazin-4(3H)-one as a white solid. (10.9 mg, 5% yield, m/z: 690.3[M+H]⁺ observed). ¹H NMR (400 MHz, Methanol-d₄): δ 7.88 (s, 1H), 7.76 (d, J=7.2 Hz, 1H), 7.66-7.64 (m, 2H), 7.52-7.44 (m, 2H), 7.37 (d, J=7.6 Hz, 1H), 7.30-7.27 (m, 3H), 4.05 (s, 1H), 1.21 (d, J=6.4 Hz, 3H).

### Example 332: 6-(2,2′-Dichloro-3′-(6-methoxy-5-(((((S)-5-oxopyrrolidin-2-yl)methyl)amino)methyl)pyridin-2-yl)-[1,1′-biphenyl]-3-yl)-2-((((1r,3r)-3-hydroxy-3-methylcyclobutyl)amino)methyl)-3-methylpyrrolo[2,1-f][1,2,4]triazin-4(3H)-one

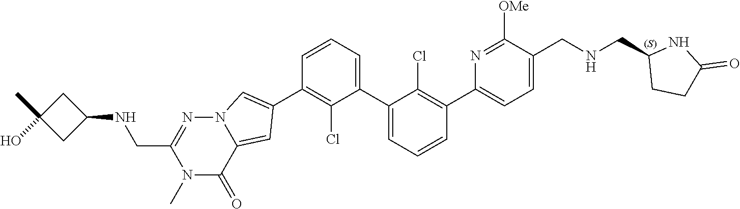

### tert-Butyl ((6-(2,2′-dichloro-3′-(2-((((1r,3r)-3-hydroxy-3-methylcyclobutyl)amino)methyl)-3-methyl-4-oxo-3,4-dihydropyrrolo[2,1-f][1,2,4]triazin-6-yl)-[1,1′-biphenyl]-3-yl)-2-methoxypyridin-3-yl)methyl)(((S)-5-oxopyrrolidin-2-yl)methyl)carbamate

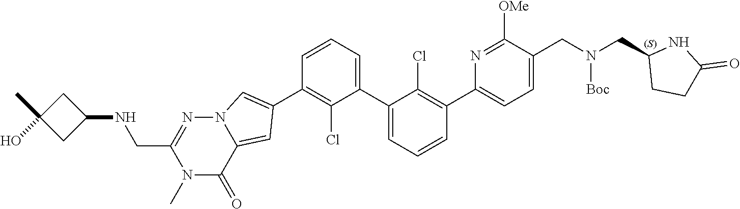

To a mixture of (S)-tert-butyl ((6-(2,2′-dichloro-3′-(2-formyl-3-methyl-4-oxo-3,4-dihydropyrrolo[2,1-f][1,2,4]triazin-6-yl)-[1,1′-biphenyl]-3-yl)-2-methoxypyridin-3-yl)methyl)((5-oxopyrrolidin-2-yl)methyl)carbamate (0.14 g, 0.19 mmol) in DCM (2 mL) was added sodium acetate (0.05 g, 0.57 mmol) and (1r,3r)-3-amino-1-methylcyclobutanol, HCl (0.04 g, 0.28 mmol). The mixture was stirred at 25° C. for 11 h. Sodium cyanoborohydride (0.02 g, 0.38 mmol) was added and the stirring continued for 1 h. The reaction mixture was concentrated under reduced pressure to give a tert-butyl ((6-(2,2′-dichloro-3′-(2-((((1r,3r)-3-hydroxy-3-methylcyclobutyl)amino)methyl)-3-methyl-4-oxo-3,4-dihydropyrrolo[2,1-f][1,2,4]triazin-6-yl)-[1,1′-biphenyl]-3-yl)-2-methoxypyridin-3-yl)methyl)(((S)-5-oxopyrrolidin-2-yl)methyl)carbamate as a yellow solid (0.2 g, crude, m/z: 816.3 [M+H]⁺ observed). The material was used without any purification.

### 6-(2,2′-Dichloro-3′-(6-methoxy-5-(((((S)-5-oxopyrrolidin-2-yl)methyl)amino)methyl)pyridin-2-yl)-[1,1′-biphenyl]-3-yl)-2-((((1r,3r)-3-hydroxy-3-methylcyclobutyl)amino)methyl)-3-methylpyrrolo[2,1-f][1,2,4]triazin-4(3H)-one

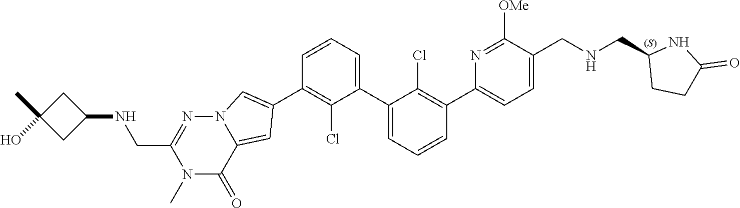

To a mixture of tert-butyl ((6-(2,2′-dichloro-3′-(2-((((1r,3r)-3-hydroxy-3-methylcyclobutyl)amino)methyl)-3-methyl-4-oxo-3,4-dihydropyrrolo[2,1-f][1,2,4]triazin-6-yl)-[1,1′-biphenyl]-3-yl)-2-methoxypyridin-3-yl)methyl)(((S)-5-oxopyrrolidin-2-yl)methyl) carbamate (0.2 g, 0.24 mmol) in DCM (5 mL) was added TFA (3 mL). The mixture was stirred at rt for 10 min. The reaction mixture was filtered and concentrated under reduced pressure and the residue was purified by reverse phase HPLC to afford 6-(2,2′-dichloro-3′-(6-methoxy-5-(((((S)-5-oxopyrrolidin-2-yl)methyl)amino)methyl)pyridin-2-yl)-[1,1′-biphenyl]-3-yl)-2-((((1r,3r)-3-hydroxy-3-methylcyclobutyl)amino)methyl)-3-methylpyrrolo[2,1-f][1,2,4]triazin-4(3H)-one as a white solid (40 mg, 20% yield, m/z: 716.3[M+H]⁺ observed). ¹H NMR (400 MHz, Methanol-d₄) δ 7.92-7.89 (m, 2H), 7.68-7.65 (m, 2H), 7.53-7.46 (m, 2H), 7.42-7.36 (m, 2H), 7.35 (d, J=2.0 Hz, 1H), 7.32 (dd, J=1.2 Hz, J=7.6 Hz, 1H), 4.38-4.29 (m, 4H), 4.12 (m, 3H), 4.09-4.01 (m, 2H), 3.53 (s, 3H), 3.29-3.20 (m, 2H), 2.51-2.27 (m, 7H), 1.97-1.88 (m, 1H), 1.44 (s, 3H).

### Example 351: (S)-6-(2,2′-Dichloro-3′-(6-methoxy-5-((((5-oxopyrrolidin-2-yl)methyl)amino)methyl)pyridin-2-yl)-[1,1′-biphenyl]-3-yl)-2-(((2-hydroxyethyl)amino)methyl)-3-methylpyrrolo[2,1-f][1,2,4]triazin-4(3H)-one

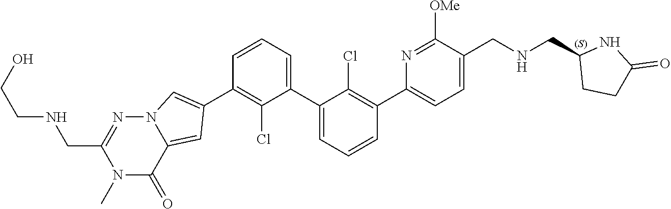

### (S)-tert-Butyl ((6-(2,2′-dichloro-3′-(2-(((2-hydroxyethyl)amino)methyl)-3-methyl-4-oxo-3,4-dihydropyrrolo[2,1-f][1,2,4]triazin-6-yl)-[1,1′-biphenyl]-3-yl)-2-methoxypyridin-3-yl)methyl)((5-oxopyrrolidin-2-yl)methyl)carbamate

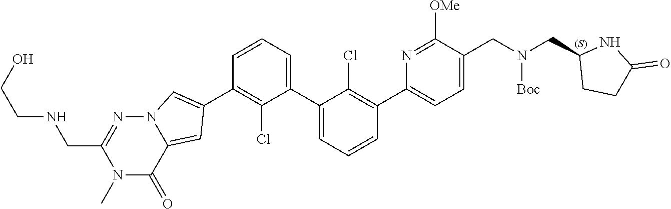

To a mixture of (S)-tert-butyl ((6-(2,2′-dichloro-3′-(2-formyl-3-methyl-4-oxo-3,4-dihydropyrrolo [2,1-f][1,2,4]triazin-6-yl)-[1,1′-biphenyl]-3-yl)-2-methoxypyridin-3-yl)methyl)((5-oxopyrrolidin-2-yl)methyl)carbamate (0.14 g, 0.19 mmol) in DCM (5 mL) was added sodium acetate (0.05 g, 0.57 mmol) and 2-aminoethanol (0.02 g, 0.29 mmol). The mixture was stirred at 25° C. for 11 h. Sodium cyanoborohydride (0.02 g, 0.38 mmol) was added and the mixture was stirred at 25° C. for 1 h. The reaction mixture was concentrated under reduced pressure to give (S)-tert-butyl ((6-(2,2′-dichloro-3′-(2-(((2-hydroxyethyl)amino)methyl)-3-methyl-4-oxo-3,4-dihydropyrrolo[2,1-f][1,2,4]triazin-6-yl)-[1,1′-biphenyl]-3-yl)-2-methoxypyridin-3-yl)methyl)((5-oxopyrrolidin-2-yl)methyl)carbamate as a yellow solid (0.2 g, crude, m/z: 776.3 [M+H]⁺ observed).

### (S)-6-(2,2′-Dichloro-3′-(6-methoxy-5-((((5-oxopyrrolidin-2-yl)methyl)amino)methyl)pyridin-2-yl)-[1,1′-biphenyl]-3-yl)-2-(((2-hydroxyethyl)amino)methyl)-3-methylpyrrolo[2,1-f][1,2,4]triazin-4(3H)-one

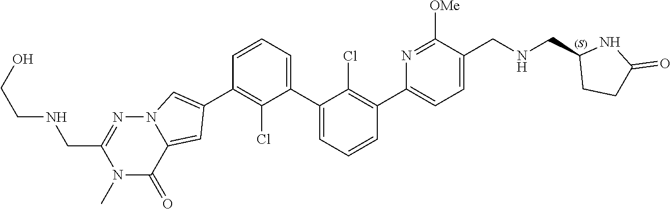

To a mixture of (S)-tert-butyl((6-(2,2′-dichloro-3′-(2-(((2-hydroxyethyl)amino)methyl)-3-methyl-4-oxo-3,4-dihydropyrrolo[2,1-f][1,2,4]]triazin-6-yl)-[1,1′-biphenyl]-3-yl)-2-methoxypyridin-3-yl)methyl)((5-oxopyrrolidin-2-yl)methyl)carbamate (0.2 g, 0.26 mmol) in DCM (2 mL) was added TFA (2 mL). The mixture was stirred at rt for 10 min. The reaction mixture was concentrated and the residue was purified by reverse phase HPLC to afford (S)-6-(2,2′-dichloro-3′-(6-methoxy-5-((((5-oxopyrrolidin-2-yl)methyl)amino)methyl)pyridin-2-yl)-[1,1′-biphenyl]-3-yl)-2-(((2-hydroxyethyl)amino)methyl)-3-methylpyrrolo[2,1-f][1,2,4]triazin-4(3H)-one as a white solid (14.1 mg, 8% yield, m/z: 676.2 [M+H]⁺ observed). ¹H NMR (400 MHz, Methanol-d₄): δ 7.88 (d, J=1.6 Hz, 1H), 7.77 (d, J=7.2 Hz, 1H), 7.65 (d, J=8 Hz, 2H), 7.53-7.44 (m, 2H), 7.38 (dd, J=1.2 Hz, J=7.2 Hz, 1H), 7.32-7.27 (m, 3H), 4.10 (s, 5H), 4.06-3.87 (m, 3H), 3.76 (t, J=5.2 Hz, 2H), 3.59 (s, 3H), 2.96 (t, J=5.6 Hz, 2H), 2.85-2.76 (m, 2H), 2.42-2.27 (m, 3H), 1.88-1.81 (m, 1H).

### Example 446: 6-[2-Chloro-3-[2-chloro-3-[6-methoxy-5-[[[(2S)-5-oxopyrrolidin-2-yl]methylamino]methyl]-2-pyridyl]phenyl]phenyl]-3-methyl-2-[(2-oxaspiro[3.3]heptan-6-ylamino)methyl] pyrrolo[2,1-f][1,2,4]triazin-4-one

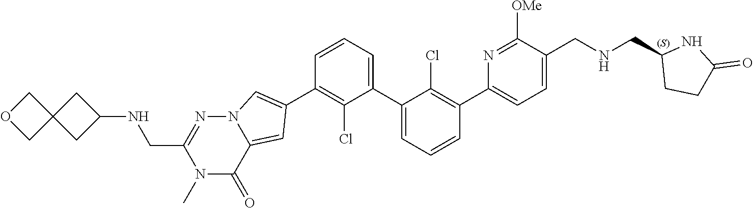

### tert-Butyl N-[[6-[2-chloro-3-[2-chloro-3-[3-methyl-2-[(2-oxaspiro[3.3]heptan-6-ylamino) methyl]-4-oxo-pyrrolo[2,1-f1[1,2,4]triazin-6-yl]phenyl]phenyl]-2-methoxy-3-pyridyl]methyl]-N-[[(2S)-5-oxopyrrolidin-2-yl]methyl]carbamate

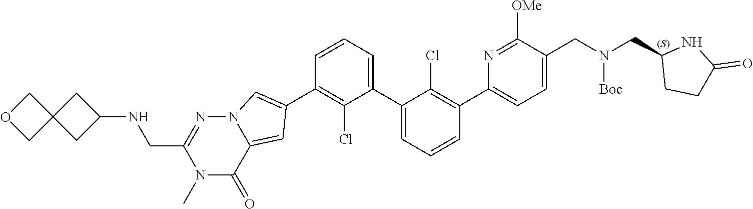

To a solution of tert-butyl N-[[6-[2-chloro-3-[2-chloro-3-[2-(hydroxymethyl)-3-methyl-4-oxo-pyrrolo[2,1-f][1,2,4]triazin-6-yl]phenyl]phenyl]-2-methoxy-3-pyridyl]methyl]-N-[[(2S)-5-oxopyrrolidin-2-yl]methyl]carbamate (20 mg, 0.03 mmol) in DCM (0.4 mL) was added mesyl chloride (2.3 uL, 0.03 mmol) and triethylamine (8 uL, 0.05 mmol). The reaction was stirred at rt for 1 h. The solvent was evaporated, and the residue was taken up in DMF (0.4 mL). Potassium carbonate, (7.4 mg, 0.05 mmol) and 2-oxaspiro[3.3]heptan-6-amine, HCl (12 mg, 0.08 mmol) were added and the reaction was stirred at rt for 12 h. The solvent was evaporated, and the residue was suspended in water. The solid formed was collected by filtration to afford tert-butyl N-[[6-[2-chloro-3-[2-chloro-3-[3-methyl-2-[(2-oxaspiro [3.3]heptan-6-ylamino)methyl]-4-oxo-pyrrolo[2,1-f][1,2,4]triazin-6-yl]phenyl]phenyl]-2-methoxy-3-pyridyl]methyl]-N-[[(2S)-5-oxopyrrolidin-2-yl]methyl]carbamate (16 mg, 70.8%). The material was used as is without any purification. m/z: 828.2 [M+H]⁺ observed.

### 6-[2-Chloro-3-[2-chloro-3-[6-methoxy-5-[[[(2S)-5-oxopyrrolidin-2-yl]methylamino]methyl]-2-pyridyl]phenyl]phenyl]-3-methyl-2-[(2-oxaspiro[3.3]heptan-6-ylamino)methyl]pyrrolo [2,1-f][1,2,4]triazin-4-one

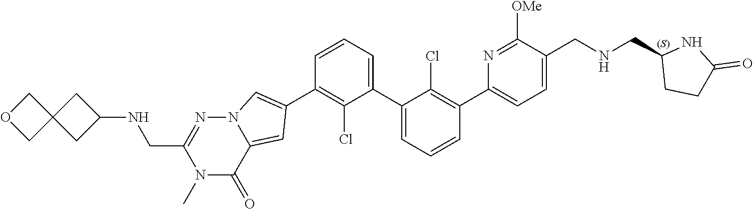

To a solution of tert-butyl N-[[6-[2-chloro-3-[2-chloro-3-[3-methyl-2-[(2-oxaspiro[3.3]heptan-6-ylamino)methyl]-4-oxo-pyrrolo[2,1-f][1,2,4]triazin-6-yl]phenyl]phenyl]-2-methoxy-3-pyridyl]methyl]-N-[[(2S)-5-oxopyrrolidin-2-yl]methyl]carbamate (16 mg, 0.02 mmol) in DCM (0.4 mL) was added TFA (74 uL, 110.06 mg, 0.97 mmol). The reaction was stirred at rt for 30 min. The solvent was evaporated and the residue was purified by reverse phase HPLC (acetonitrile:water) to afford 6-[2-chloro-3-[2-chloro-3-[6-methoxy-5-[[[(2S)-5-oxopyrrolidin-2-yl]methylamino]methyl]-2-pyridyl]phenyl]phenyl]-3-methyl-2-[(2-oxaspiro[3.3]heptan-6-ylamino)methyl]pyrrolo[2,1-f][1,2,4]triazin-4-one (3.4 mg, 24.2%). m/z: 728.2 [M+H]⁺ observed. ¹H NMR (400 MHz, Methanol-d₄): δ 7.85 (s, 1H), 7.79 (d, J=7.4 Hz, 1H), 7.63 (d, J=7.7 Hz, 2H), 7.46 (dt, J=18.0, 7.6 Hz, 2H), 7.38-7.34 (m, 1H), 7.31-7.24 (m, 3H), 4.71 (s, 2H), 4.61 (s, 2H), 4.04 (s, 3H), 4.00 (s, 2H), 3.89 (d, J=24.5 Hz, 2H), 3.75 (s, 1H), 3.58 (s, 1H), 3.56 (s, 3H), 3.22 (s, 1H), 2.89 (s, 2H), 2.58 (s, 1H), 2.42-2.27 (m, 4H), 2.06 (s, 1H), 1.86 (dd, J=34.2, 24.0 Hz, 2H).

### Example 447: 6-[2-Chloro-3-[2-chloro-3-[6-methoxy-5-[[[(2S)-5-oxopyrrolidin-2-yl]methylamino]methyl]-2-pyridyl]phenyl]phenyl]-3-methyl-2-(2-oxa-6-azaspiro[3.3]heptan-6-ylmethyl)pyrrolo[2,1-f][1,2,4]triazin-4-one

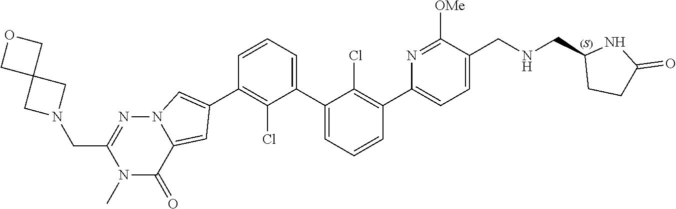

### tert-butyl N-[[6-[2-Chloro-3-[2-chloro-3-[3-methyl-2-(2-oxa-6-azaspiro[3.3]heptan-6-ylmethyl)-4-oxo-pyrrolo[2,1-f][1,2,4]triazin-6-yl]phenyl]phenyl]-2-methoxy-3-pyridyl]methyl]-N-[[(2S)-5-oxopyrrolidin-2-yl]methyl]carbamate

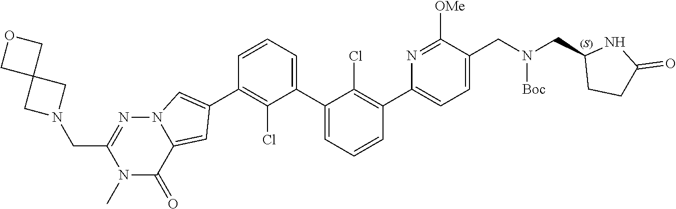

To a solution of tert-butyl N-[[6-[2-chloro-3-[2-chloro-3-[2-(hydroxymethyl)-3-methyl-4-oxo-pyrrolo[2,1-f1][1,2,4]triazin-6-yl]phenyl]phenyl]-2-methoxy-3-pyridyl]methyl]-N-[[(2S)-5-oxopyrrolidin-2-yl]methyl]carbamate (20 mg, 0.03 mmol) in DCM (0.5 mL), triethylamine (7.6 uL, 0.05 mmol) and mesyl chloride, (2.3 uL, 0.03 mmol) was added. The reaction was stirred at rt for 1 h. The solvent was evaporated and the residue was suspended in THF (0.4 mL) followed by addition of potassium carbonate (7.4 mg, 0.05 mmol) and 2-oxa-6-azaspiro[3.3]heptane, (8 mg, 0.08 mmol). The mixture was stirred at rt for 12 h. The solvent was evaporated, and the residue was suspended in water. The solid formed was collected to afford tert-butyl N-[[6-[2-chloro-3-[2-chloro-3-[3-methyl-2-(2-oxa-6-azaspiro[3.3]heptan-6-ylmethyl)-4-oxo-pyrrolo[2,1-f][1,2,4]triazin-6-yl]phenyl]phenyl]-2-methoxy-3-pyridyl]methyl]-N-[[(2S)-5-oxopyrrolidin-2-yl]methyl]carbamate (20 mg, 90.0%). m/z: 814.4 [M+H]⁺ observed.

### 6-[2-Chloro-3-[2-chloro-3-[6-methoxy-5-[[[(2S)-5-oxopyrrolidin-2-yl]methylamino]methyl]-2-pyridyl]phenyl]phenyl]-3-methyl-2-(2-oxa-6-azaspiro[3.3]heptan-6-ylmethyl)pyrrolo[2,1-f][1,2,4]triazin-4-one

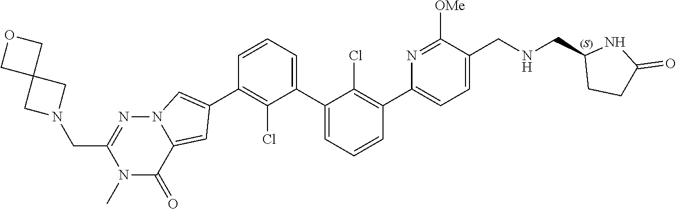

To a solution of tert-butyl N-[[6-[2-chloro-3-[2-chloro-3-[3-methyl-2-(2-oxa-6-azaspiro[3.3]heptan-6-ylmethyl)-4-oxo-pyrrolo[2,1-f][1,2,4]triazin-6-yl]phenyl]phenyl]-2-methoxy-3-pyridyl]methyl]-N-[[(2S)-5-oxopyrrolidin-2-yl]methyl]carbamate (20 mg, 0.02 mmol) in DCM (0.1 mL) was added TFA (38 uL, 56 mg, 0.49 mmol). The reaction was stirred at rt for 1 h. The solvent was evaporated and the residue was purified by reverse phase HPLC (acetonitrile:water) to afford 6-[2-chloro-3-[2-chloro-3-[6-methoxy-5-[[[(2S)-5-oxopyrrolidin-2-yl]methylamino]methyl]-2-pyridyl]phenyl]phenyl]-3-methyl-2-(2-oxa-6-azaspiro[3.3]heptan-6-ylmethyl)pyrrolo[2,1-f][1,2,4]triazin-4-one (3.7 mg, 21.1%). m/z: 714.3 [M+H]⁺ observed. ¹H NMR (400 MHz, Methanol-d₄) δ 8.30 (s, 3H), 7.88-7.81 (m, 2H), 7.63 (ddd, J=7.8, 3.3, 1.6 Hz, 2H), 7.46 (dt, J=22.0, 7.7 Hz, 2H), 7.41-7.30 (m, 2H), 7.30-7.23 (m, 2H), 4.78 (s, 4H), 4.21 (d, J=2.5 Hz, 2H), 4.07 (s, 3H), 4.01 (d, J=9.1 Hz, 2H), 3.89 (s, 2H), 3.77 (s, 4H), 3.51 (s, 3H), 3.11 (t, J=5.6 Hz, 2H), 2.36 (t, J=8.4 Hz, 3H).

### Example 448: 6-[2-Chloro-3-[2-chloro-3-[6-methoxy-5-[[[(2S)-5-oxopyrrolidin-2-yl]methylamino]methyl]-2-pyridyl]phenyl]phenyl]-3-methyl-2-[(6-oxo-2,5-diazaspiro[3.4]octan-2-yl)methyl]pyrrolo [2,1-f][1,2,4]triazin-4-one

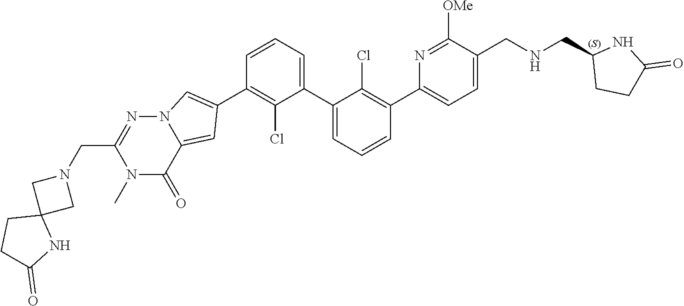

### tert-Butyl N-[[6-[2-chloro-3-[2-chloro-3-[3-methyl-4-oxo-2-[(6-oxo-2,5-diazaspiro[3.4]octan-2-yl)methyl]pyrrolo[2,1-f][1,2,4]triazin-6-yl]phenyl]phenyl]-2-methoxy-3-pyridyl]methyl]-N-[[(2S)-5-oxopyrrolidin-2-yl]methyl]carbamate

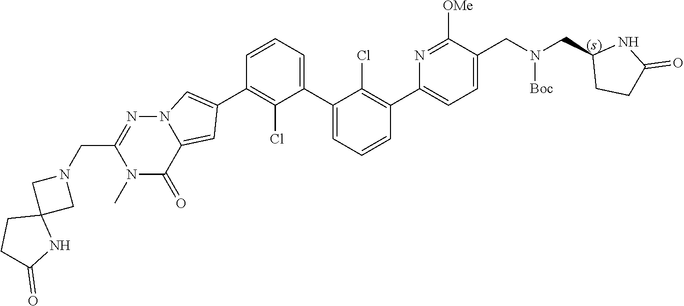 [SMILES: COc1nc(-c2cccc(-c3cccc(-c4cc5c(=O)n(C)c(CN6CC7(CCC(=O)N7)C6)nn5c4)c3Cl)c2Cl)ccc1CN(C[C@@H]1CCC(=O)N1)C(=O)OC(C)(C)C]

To a solution of [6-[3-[3-[5-[[tert-butoxycarbonyl-[[(2S)-5-oxopyrrolidin-2-yl]methyl]amino]methyl]-6-methoxy-2-pyridyl]-2-chloro-phenyl]-2-chloro-phenyl]-3-methyl-4-oxo-pyrrolo[2,1-f][1,2,4]triazin-2-yl]methyl methanesulfonate (16 mg, 0.02 mmol) in acetonitrile (0.4 mL), potassium carbonate (8.1 mg, 0.06 mmol) and 2,5-diazaspiro[3.4]octan-6-one, HCl (9.6 mg, 0.06 mmol) was added. The reaction was stirred at 60° C. for 30 min. The solvent was evaporated, and the residue was suspended in water. The solid formed was collected to afford tert-butyl N-[[6-[2-chloro-3-[2-chloro-3-[3-methyl-4-oxo-2-[(6-oxo-2,5-diazaspiro[3.4]octan-2-yl)methyl]pyrrolo[2,1-f][1,2,4]triazin-6-yl]phenyl]phenyl]-2-methoxy-3-pyridyl]methyl]-N-[[(2S)-5-oxopyrrolidin-2-yl]methyl]carbamate (9 mg, 54.2%). The material was used as is for the next step. m/z: 841.3 [M+H]⁺ observed.

### 6-[2-Chloro-3-[2-chloro-3-[6-methoxy-5-[[[(2S)-5-oxopyrrolidin-2-yl]methylamino]methyl]-2-pyridyl]phenyl]phenyl]-3-methyl-2-[(6-oxo-2,5-diazaspiro[3.4]octan-2-yl)methyl]pyrrolo [2,1-f][1,2,4]triazin-4-one

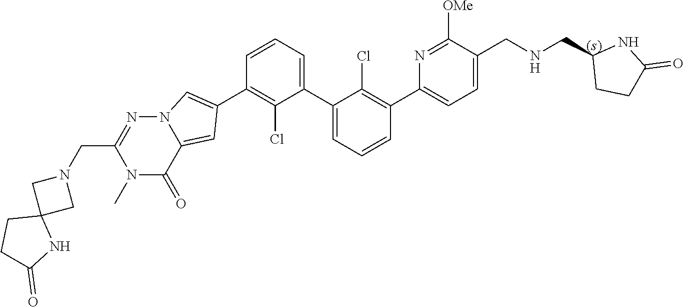 [SMILES: COc1nc(-c2cccc(-c3cccc(-c4cc5c(=O)n(C)c(CN6CC7(CCC(=O)N7)C6)nn5c4)c3Cl)c2Cl)ccc1CNC[C@@H]1CCC(=O)N1]

To a solution of tert-butyl N-[[6-[2-chloro-3-[2-chloro-3-[3-methyl-4-oxo-2-[(6-oxo-2,5-diazaspiro[3.4]octan-2-yl)methyl]pyrrolo[2,1-f][1,2,4]triazin-6-yl]phenyl]phenyl]-2-methoxy-3-pyridyl]methyl]-N-[[(2S)-5-oxopyrrolidin-2-yl]methyl]carbamate (9 mg, 0.01 mmol) in DCM (0.4 mL) was added TFA (17 uL, 0.22 mmol). The reaction was stirred at rt for 30 min. The solvent was evaporated and the residue was purified by reverse phase HPLC (acetonitrile:water) to afford 6-[2-chloro-3-[2-chloro-3-[6-methoxy-5-[[[(2S)-5-oxopyrrolidin-2-yl]methylamino] methyl]-2-pyridyl]phenyl]phenyl]-3-methyl-2-[(6-oxo-2,5-diazaspiro[3.4]octan-2-yl)methyl] pyrrolo[2,1-f][1,2,4]triazin-4-one (5 mg, 63.3%). m/z: 741.3 [M+H]⁺ observed. ¹H NMR (400 MHz, Methanol-d₄): δ 8.41 (s, 1H), 7.88-7.78 (m, 2H), 7.66-7.58 (m, 2H), 7.46 (dt, J=20.3, 7.7 Hz, 2H), 7.39-7.21 (m, 4H), 4.09 (s, 2H), 4.06 (s, 3H), 3.98 (d, J=18.9 Hz, 1H), 3.77 (s, 2H), 3.59 (d, J=3.7 Hz, 5H), 3.41 (d, J=7.7 Hz, 2H), 2.98 (t, J=6.0 Hz, 2H), 2.45-2.28 (m, 8H).

### Example 449: 6-[2-Chloro-3-[2-chloro-3-[6-methoxy-5-[[[(2S)-5-oxopyrrolidin-2-yl]methylamino] methyl]-2-pyridyl]phenyl]phenyl]-3-methyl-2-[[(6-oxo-5-azaspiro[3.4]octan-2-yl)amino] methyl] pyrrolo[2,1-f][1,2,4]triazin-4-one

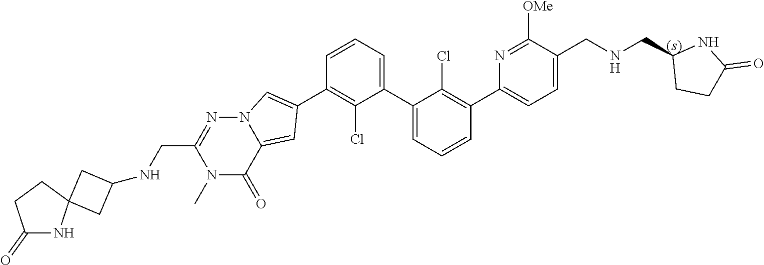 [SMILES: COc1nc(-c2cccc(-c3cccc(-c4cc5c(=O)n(C)c(CNC6CC7(CCC(=O)N7)C6)nn5c4)c3Cl)c2Cl)ccc1CNC[C@@H]1CCC(=O)N1]

### tert-Butyl N-[[6-[2-chloro-3-[2-chloro-3-[3-methyl-4-oxo-2-[[(6-oxo-5-azaspiro[3.4]octan-2-yl)amino]methyl]pyrrolo[2,1-f1[1,2,4]triazin-6-yl]phenyl]phenyl]-2-methoxy-3-pyridyl] methyl]-N-[[(2S)-5-oxopyrrolidin-2-yl]methyl]carbamate

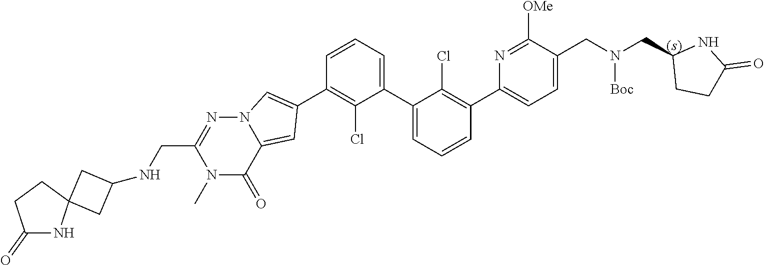 [SMILES: COc1nc(-c2cccc(-c3cccc(-c4cc5c(=O)n(C)c(CNC6CC7(CCC(=O)N7)C6)nn5c4)c3Cl)c2Cl)ccc1CN(C[C@@H]1CCC(=O)N1)C(=O)OC(C)(C)C]

To a solution of tert-butyl N-[[6-[2-chloro-3-[2-chloro-3-[2-(hydroxymethyl)-3-methyl-4-oxo-pyrrolo[2,1-f][1,2,4]triazin-6-yl]phenyl]phenyl]-2-methoxy-3-pyridyl]methyl]-N-[[(2S)-5-oxopyrrolidin-2-yl]methyl]carbamate (20 mg, 0.03 mmol) in DCM (0.4 mL) was added mesyl chloride (2.3 uL, 0.03 mmol) and triethylamine (7.6 uL, 0.05 mmol). The reaction was stirred at rt for 1 h. The solvent was evaporated, and the residue was dissolved in DMF (0.4 mL). Potassium carbonate (7.4 mg, 0.05 mmol) and 2-amino-5-azaspiro[3.4]octan-6-one (11.46 mg, 0.08 mmol) were added and the reaction was stirred at 60° C. for 16 h. The solvent was evaporated, and the residue was suspended in water. The solid formed was collected by filtration to afford tert-butyl N-[[6-[2-chloro-3-[2-chloro-3-[3-methyl-4-oxo-2-[[(6-oxo-5-azaspiro[3.4]octan-2-yl)amino]methyl]pyrrolo[2,1-f][1,2,4]triazin-6-yl]phenyl]phenyl]-2-methoxy-3-pyridyl]methyl]-N-[[(2S)-5-oxopyrrolidin-2-yl]methyl]carbamate (18 mg, 77.2%). The material was used as is without any purification. m/z: 855.3 [M+H]⁺ observed.

### 6-[2-Chloro-3-[2-chloro-3-[6-methoxy-5-[[[rac-(2S)-5-oxopyrrolidin-2-yl]methylamino]methyl]-2-pyridyl]phenyl]phenyl]-3-methyl-2-[[(6-oxo-5-azaspiro[3.4]octan-2-yl)amino]methyl]pyrrolo[2,1-f][1,2,4]triazin-4-one

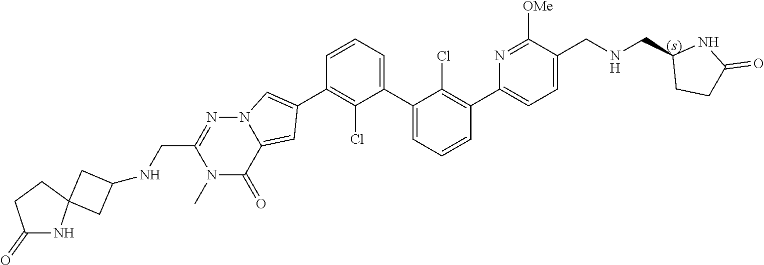 [SMILES: COc1nc(-c2cccc(-c3cccc(-c4cc5c(=O)n(C)c(CNC6CC7(CCC(=O)N7)C6)nn5c4)c3Cl)c2Cl)ccc1CNC[C@@H]1CCC(=O)N1]

To a solution of tert-butyl N-[[6-[2-chloro-3-[2-chloro-3-[3-methyl-4-oxo-2-[[(6-oxo-5-azaspiro[3.4]octan-2-yl)amino]methyl]pyrrolo[2,1-f][1,2,4]triazin-6-yl]phenyl]phenyl]-2-methoxy-3-pyridyl]methyl]-N-[[(2S)-5-oxopyrrolidin-2-yl]methyl]carbamate (18 mg, 0.02 mmol) in DCM (0.1 mL) was added TFA (32 uL, 0.42 mmol). The reaction was stirred at rt for 1 h. The solvent was evaporated and the residue was purified by reverse phase HPLC (acetonitrile:water) to afford 6-[2-chloro-3-[2-chloro-3-[6-methoxy-5-[[[rac-(2S)-5-oxopyrrolidin-2-yl]methylamino]methyl]-2-pyridyl]phenyl]phenyl]-3-methyl-2-[[(6-oxo-5-azaspiro[3.4]octan-2-yl)amino]methyl]pyrrolo[2,1-f][1,2,4]triazin-4-one (5.4 mg, 34.0%). m/z: 755.3 [M+H]⁺ observed. ¹H NMR (400 MHz, Methanol-d₄): δ7.85 (dd, J=5.8, 1.9 Hz, 1H), 7.76 (d, J=7.5 Hz, 1H), 7.66-7.59 (m, 2H), 7.46 (dt, J=16.0, 7.6 Hz, 2H), 7.35 (dd, J=7.6, 1.7 Hz, 1H), 7.27 (dd, J=7.3, 2.0 Hz, 3H), 4.03 (s, 3H), 3.95-3.85 (m, 5H), 3.65 (d, J=7.4 Hz, OH), 3.58 (d, J=1.2 Hz, 4H), 3.31-3.22 (m, 2H), 2.80 (t, J=6.3 Hz, 1H), 2.65-2.55 (m, OH), 2.39-2.26 (m, 2H), 2.29-2.14 (m, 3H), 1.95 (t, J=10.1 Hz, 1H), 1.88-1.78 (m, 1H).

### Example 450: 6-[2-Chloro-3-[2-chloro-3-[6-methoxy-5-[[[rac-(2S)-5-oxopyrrolidin-2-yl]methylamino] methyl]-2-pyridyl]phenyl]phenyl]-2-[[(4,4-difluoro-5-oxo-pyrrolidin-2-yl)methylamino] methyl]-3-methyl-pyrrolo[2,1-f][1,2,4]triazin-4-one

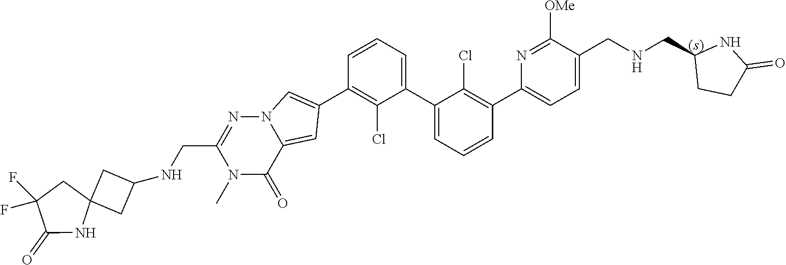 [SMILES: COc1nc(-c2cccc(-c3cccc(-c4cc5c(=O)n(C)c(CNC6CC7(C6)CC(F)(F)C(=O)N7)nn5c4)c3Cl)c2Cl)ccc1CNC[C@@H]1CCC(=O)N1]

### tert-butyl N-[[6-[2-Chloro-3-[2-chloro-3-[2-[[(4,4-difluoro-5-oxo-pyrrolidin-2-yl)methylamino]methyl]-3-methyl-4-oxo-pyrrolo[2,1-f][1,2,4]triazin-6-yl]phenyl]phenyl]-2-methoxy-3-pyridyl]methyl]-N-[[(2S)-5-oxopyrrolidin-2-yl]methyl]carbamate

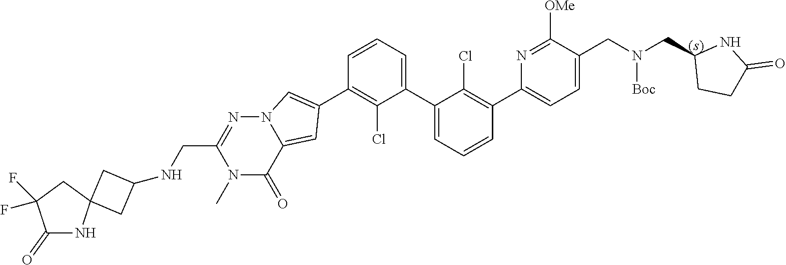 [SMILES: COc1nc(-c2cccc(-c3cccc(-c4cc5c(=O)n(C)c(CNC6CC7(C6)CC(F)(F)C(=O)N7)nn5c4)c3Cl)c2Cl)ccc1CN(C[C@@H]1CCC(=O)N1)C(=O)OC(C)(C)C]

To a solution of tert-butyl N-[[6-[2-chloro-3-[2-chloro-3-[2-(hydroxymethyl)-3-methyl-4-oxo-pyrrolo[2,1-f1][1,2,4]triazin-6-yl]phenyl]phenyl]-2-methoxy-3-pyridyl]methyl]-N-[[(2S)-5-oxopyrrolidin-2-yl]methyl]carbamate (20 mg, 0.03 mmol) in DCM (0.1 mL) was added mesyl chloride (2.3 uL, 0.03 mmol) and triethylamine (7.6 uL, 0.05 mmol). The reaction was stirred at rt for 1 h. The solvent was evaporated, and the residue was suspended in THE (0.4 mL). Potassium carbonate (7 mg, 0.05 mmol) and 5-(aminomethyl)-3,3-difluoro-pyrrolidin-2-one (12 mg, 0.08 mmol) was added, and the reaction was stirred at 60° C. for 2h. The solvent was evaporated, and the residue was suspended in water. The solid formed was collected by filtration to afford tert-butyl N-[[6-[2-chloro-3-[2-chloro-3-[2-[[(4,4-difluoro-5-oxo-pyrrolidin-2-yl)methylamino]methyl]-3-methyl-4-oxo-pyrrolo[2,1-f][1,2,4]triazin-6-yl]phenyl]phenyl]-2-methoxy-3-pyridyl]methyl]-N-[[(2S)-5-oxopyrrolidin-2-yl]methyl]carbamate (21 mg, 89.0%). used as is for the next step. m/z: 865.2 [M+H]⁺ observed.

### 6-[2-Chloro-3-[2-chloro-3-[6-methoxy-5-[[[rac-(2S)-5-oxopyrrolidin-2-yl]methylamino]methyl]-2-pyridyl]phenyl]phenyl]-2-[[(4,4-difluoro-5-oxo-pyrrolidin-2-yl)methylamino]methyl]-3-methyl-pyrrolo[2,1-f]1[1,2,4]triazin-4-one

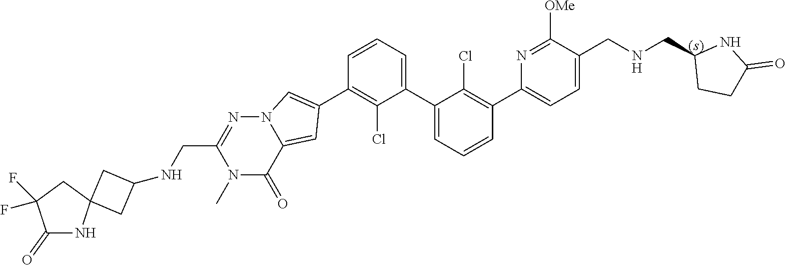 [SMILES: COc1nc(-c2cccc(-c3cccc(-c4cc5c(=O)n(C)c(CNC6CC7(C6)CC(F)(F)C(=O)N7)nn5c4)c3Cl)c2Cl)ccc1CNC[C@@H]1CCC(=O)N1]

To a solution of tert-butyl N-[[6-[2-chloro-3-[2-chloro-3-[2-[[(4,4-difluoro-5-oxo-pyrrolidin-2-yl)methylamino]methyl]-3-methyl-4-oxo-pyrrolo[2,1-f][1,2,4]triazin-6-yl]phenyl]phenyl]-2-methoxy-3-pyridyl]methyl]-N-[[(2S)-5-oxopyrrolidin-2-yl]methyl]carbamate (21 mg, 0.02 mmol) in DCM (0.4 mL) was added TFA (93 uL, 1.21 mmol). The reaction was stirred at rt for 30 min. The solvent was evaporated and the residue was purified by reverse phase HPLC (acetonitrile:water) to afford 6-[2-chloro-3-[2-chloro-3-[6-methoxy-5-[[[rac-(2S)-5-oxopyrrolidin-2-yl]methylamino]methyl]-2-pyridyl]phenyl]phenyl]-2-[[(4,4-difluoro-5-oxo-pyrrolidin-2-yl)methylamino]methyl]-3-methyl-pyrrolo[2,1-f][1,2,4]triazin-4-one (8.00 mg, 43.1%). m/z: 765.2 [M+H]⁺ observed. ¹H NMR (400 MHz, CD₃OD): δ7.84 (d, J=1.9 Hz, 1H), 7.79 (d, J=7.5 Hz, 1H), 7.63 (dd, J=7.6, 1.7 Hz, 2H), 7.52-7.41 (m, 2H), 7.36 (dd, J=7.6, 1.7 Hz, 1H), 7.30 (d, J=7.5 Hz, 1H), 7.28-7.24 (m, 2H), 4.05 (s, 3H), 4.04 (d, J=2.9 Hz, 2H), 3.94 (d, J=3.0 Hz, 3H), 3.84 (s, 1H), 3.59 (s, 3H), 2.95-2.86 (m, 3H), 2.82 (dd, J=12.4, 6.7 Hz, 1H), 2.54 (s, 1H), 2.40-2.29 (m, 4H).

### Example 451: 6-[2-Chloro-3-[2-chloro-3-[6-methoxy-5-[[[(2S)-5-oxopyrrolidin-2-yl]methylamino]methyl]-2-pyridyl]phenyl]phenyl]-3-methyl-2-[[2-(5-oxopyrrolidin-2-yl)ethylamino]methyl]pyrrolo [2,1-f][1,2,4]triazin-4-one

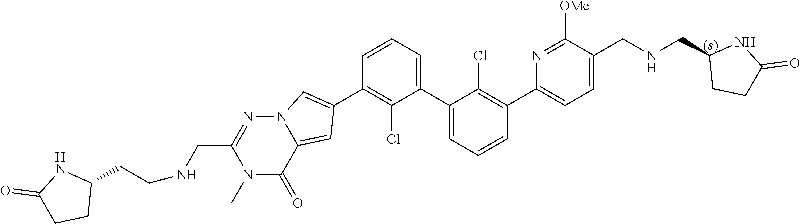 [SMILES: COc1nc(-c2cccc(-c3cccc(-c4cc5c(=O)n(C)c(CNCC[C@@H]6CCC(=O)N6)nn5c4)c3Cl)c2Cl)ccc1CNC[C@@H]1CCC(=O)N1]

### tert-butyl N-[[6-[2-Chloro-3-[2-chloro-3-[3-methyl-4-oxo-2-[[2-[(2S)-5-oxopyrrolidin-2-yl]ethylamino]methyl]pyrrolo[2,1-f][1,2,4]triazin-6-yl]phenyl]phenyl]-2-methoxy-3-pyridyl]methyl]-N-[[(2S)-5-oxopyrrolidin-2-yl]methyl]carbamate

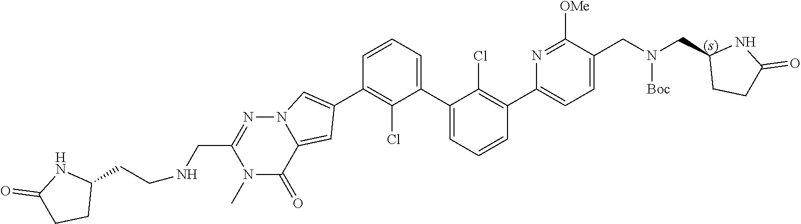 [SMILES: COc1nc(-c2cccc(-c3cccc(-c4cc5c(=O)n(C)c(CNCC[C@@H]6CCC(=O)N6)nn5c4)c3Cl)c2Cl)ccc1CN(C[C@@H]1CCC(=O)N1)C(=O)OC(C)(C)C]

To a solution of tert-butyl N-[[6-[2-chloro-3-[2-chloro-3-[2-(hydroxymethyl)-3-methyl-4-oxo-pyrrolo[2,1-f1][1,2,4]triazin-6-yl]phenyl]phenyl]-2-methoxy-3-pyridyl]methyl]-N-[[(2S)-5-oxopyrrolidin-2-yl]methyl]carbamate (20 mg, 0.03 mmol) in DCM (0.1 mL) was added mesyl chloride (2.3 uL, 0.03 mmol) and triethylamine (7.6 uL, 0.05 mmol). The reaction was stirred at rt for 1 h. The solvent was evaporated, and the residue was suspended in DMF (0.4 mL). Potassium carbonate (7.4 mg, 0.05 mmol) and (5S)-5-(2-aminoethyl)pyrrolidin-2-one (10.48 mg, 0.08 mmol) was added and the reaction was stirred at 60° C. for 12 h. The solvent was evaporated, and the residue was suspended in water. The solid formed was collected by filtration to afford tert-butyl N-[[6-[2-chloro-3-[2-chloro-3-[3-methyl-4-oxo-2-[[2-[(2S)-5-oxopyrrolidin-2-yl]ethylamino]methyl]pyrrolo[2,1-f][1,2,4]triazin-6-yl]phenyl]phenyl]-2-methoxy-3-pyridyl]methyl]-N-[[(2S)-5-oxopyrrolidin-2-yl]methyl]carbamate (15 mg, 65.2%). The material was used as is without any purification. m/z: 843.3 [M+H]⁺ observed.

### 6-[2-chloro-3-[2-Chloro-3-[6-methoxy-5-[[[(2S)-5-oxopyrrolidin-2-yl]methylamino]methyl]-2-pyridyl]phenyl]phenyl]-3-methyl-2-[[2-(5-oxopyrrolidin-2-yl)ethylamino]methyl]pyrrolo [2,1-f][1,2,4]triazin-4-one

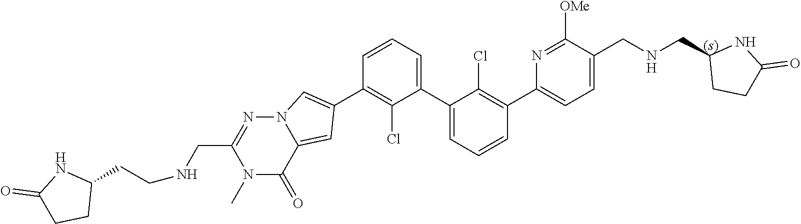 [SMILES: COc1nc(-c2cccc(-c3cccc(-c4cc5c(=O)n(C)c(CNCC[C@@H]6CCC(=O)N6)nn5c4)c3Cl)c2Cl)ccc1CNC[C@@H]1CCC(=O)N1]

To a solution of tert-butyl N-[[6-[2-chloro-3-[2-chloro-3-[3-methyl-4-oxo-2-[[2-[(2S)-5-oxopyrrolidin-2-yl]ethylamino]methyl]pyrrolo[2,1-f][1,2,4]triazin-6-yl]phenyl]phenyl]-2-methoxy-3-pyridyl]methyl]-N-[[(2S)-5-oxopyrrolidin-2-yl]methyl]carbamate (15 mg, 0.02 mmol) in DCM (0.4 mL) was added TFA (68 uL, 0.89 mmol). The reaction was stirred at rt for 30 min. The solvent was evaporated and the residue was purified by reverse phase HPLC (acetonitrile:water) to afford 6-[2-chloro-3-[2-chloro-3-[6-methoxy-5-[[[(2S)-5-oxopyrrolidin-2-yl]methylamino]methyl]-2-pyridyl]phenyl]phenyl]-3-methyl-2-[[2-(5-oxopyrrolidin-2-yl)ethylamino]methyl]pyrrolo[2,1-f][1,2,4]triazin-4-one (8.3 mg, 63%). m/z 745.0 [M+H]⁺ observed. ¹H NMR (400 MHz, Methanol-d₄): δ 7.88 (s, 1H), 7.81 (d, J=7.5 Hz, 1H), 7.68-7.57 (m, 2H), 7.46 (dt, J=18.3, 7.6 Hz, 2H), 7.35 (dd, J=7.6, 1.8 Hz, 1H), 7.33-7.23 (m, 3H), 4.08 (s, 4H), 4.05-4.02 (m, 3H), 3.97 (d, J=14.1 Hz, 1H), 3.80 (d, J=6.9 Hz, 1H), 3.55 (s, 3H), 2.95 (d, J=18.7 Hz, 4H), 2.45-2.23 (m, 7H), 1.97-1.69 (m, 4H).

### Example 460: 2-[(2-Acetyl-2,6-diazaspiro[3.3]heptan-6-yl)methyl]-6-[2-chloro-3-[2-chloro-3-[6-methoxy-5-[[[(2S)-5-oxopyrrolidin-2-yl]methylamino]methyl]-2-pyridyl]phenyl]phenyl]-3-methyl-pyrrolo[2,1-f][1,2,4]triazin-4-one

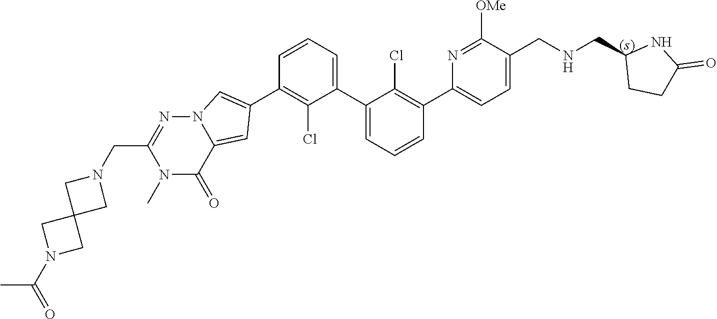 [SMILES: COc1nc(-c2cccc(-c3cccc(-c4cc5c(=O)n(C)c(CN6CC7(C6)CN(C(C)=O)C7)nn5c4)c3Cl)c2Cl)ccc1CNC[C@@H]1CCC(=O)N1]

### tert-Butyl N-[[6-[3-[3-[2-[(2-acetyl-2,6-diazaspiro[3.3]heptan-6-yl)methyl]-3-methyl-4-oxo-pyrrolo[2,1-f][1,2,4]triazin-6-yl]-2-chloro-phenyl]-2-chloro-phenyl]-2-methoxy-3-pyridyl]methyl]-N-[[(2S)-5-oxopyrrolidin-2-yl]methyl]carbamate

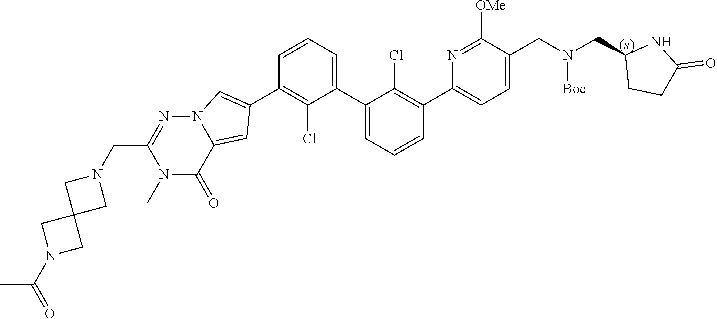 [SMILES: COc1nc(-c2cccc(-c3cccc(-c4cc5c(=O)n(C)c(CN6CC7(C6)CN(C(C)=O)C7)nn5c4)c3Cl)c2Cl)ccc1CN(C[C@@H]1CCC(=O)N1)C(=O)OC(C)(C)C]

To a solution of tert-butyl N-[[6-[2-chloro-3-[2-chloro-3-[2-(hydroxymethyl)-3-methyl-4-oxo-pyrrolo[2,1-f][1,2,4]triazin-6-yl]phenyl]phenyl]-2-methoxy-3-pyridyl]methyl]-N-[[(2S)-5-oxopyrrolidin-2-yl]methyl]carbamate (20 mg, 0.03 mmol) in DCM (0.1 mL) was added mesyl chloride (2.3 uL, 0.03 mmol) and triethylamine (7.6 uL, 0.05 mmol). The reaction was stirred at rt for 1 h. The solvent was evaporated, and the residue was suspended in DMF (0.4 mL). To this suspension, 1-(2,6-diazaspiro[3.3]heptan-2-yl)ethenone, HCl (13 mg, 0.07 mmol) and potassium carbonate (10 mg, 0.07 mmol) were added. The reaction was stirred at rt for 1 h. The solvent was evaporated, and the residue was suspended in water. The solid formed was collected by filtration to afford tert-butyl N-[[6-[3-[3-[2-[(2-acetyl-2,6-diazaspiro[3.3]heptan-6-yl)methyl]-3-methyl-4-oxo-pyrrolo[2,1-f][1,2,4]triazin-6-yl]-2-chloro-phenyl]-2-chloro-phenyl]-2-methoxy-3-pyridyl]methyl]-N-[[(2S)-5-oxopyrrolidin-2-yl]methyl]carbamate (18 mg, 85.4%). The material was used as is without any purification. m/z: 855.2 [M+H]⁺ observed.

### 2-[(2-Acetyl-2,6-diazaspiro[3.3]heptan-6-yl)methyl]-6-[2-chloro-3-[2-chloro-3-[6-methoxy-5-[[[(2S)-5-oxopyrrolidin-2-yl]methylamino]methyl]-2-pyridyl]phenyl]phenyl]-3-methyl-pyrrolo[2,1-f]1[1,2,4]triazin-4-one

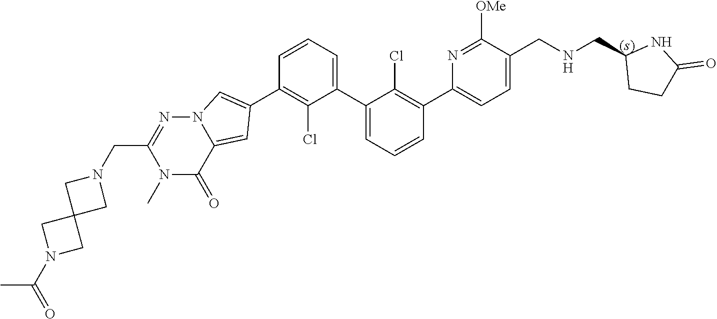 [SMILES: COc1nc(-c2cccc(-c3cccc(-c4cc5c(=O)n(C)c(CN6CC7(C6)CN(C(C)=O)C7)nn5c4)c3Cl)c2Cl)ccc1CNC[C@@H]1CCC(=O)N1]

To a solution of tert-butyl N-[[6-[3-[3-[2-[(2-acetyl-2,6-diazaspiro[3.3]heptan-6-yl)methyl]-3-methyl-4-oxo-pyrrolo[2,1-f][1,2,4]triazin-6-yl]-2-chloro-phenyl]-2-chloro-phenyl]-2-methoxy-3-pyridyl]methyl]-N-[[(2S)-5-oxopyrrolidin-2-yl]methyl]carbamate (18 mg, 0.02 mmol) in DCM (0.4 mL) was added TFA (80 uL, 1.05 mmol). The reaction was stirred at rt for 30 min. The solvent was evaporated and the residue was purified by reverse phase HPLC (acetonitrile:water) to afford 2-[(2-acetyl-2,6-diazaspiro[3.3]heptan-6-yl)methyl]-6-[2-chloro-3-[2-chloro-3-[6-methoxy-5-[[[(2S)-5-oxopyrrolidin-2-yl]methylamino]methyl]-2-pyridyl]phenyl]phenyl]-3-methyl-pyrrolo[2,1-f][1,2,4]triazin-4-one (5.6 mg, 35.2%). m/z: 747.3 [M+H]⁺ observed. ¹H NMR (400 MHz, Methanol-d₄): δ 8.33 (s, 1H), 7.86-7.80 (m, 2H), 7.63 (ddd, J=7.8, 3.6, 1.7 Hz, 2H), 7.47 (dt, J=21.4, 7.7 Hz, 2H), 7.41-7.29 (m, 2H), 7.30-7.23 (m, 2H), 4.32 (s, 2H), 4.16 (d, J=2.6 Hz, 2H), 4.07 (s, 5H), 4.02-3.94 (m, 1H), 3.81 (s, 2H), 3.68-3.58 (m, 4H), 3.55 (s, 3H), 3.13-2.99 (m, 2H), 2.46-2.31 (m, 3H), 1.84 (s, 3H).

### Example 461: 6-[2-Chloro-3-[2-chloro-3-[6-methoxy-5-[[[(2S)-5-oxopyrrolidin-2-yl]methylamino]methyl]-2-pyridyl]phenyl]phenyl]-2-[[3-(1-hydroxy-1-methyl-ethyl)azetidin-1-yl]methyl]-3-methyl-pyrrolo[2,1-f][1,2,4]triazin-4-one

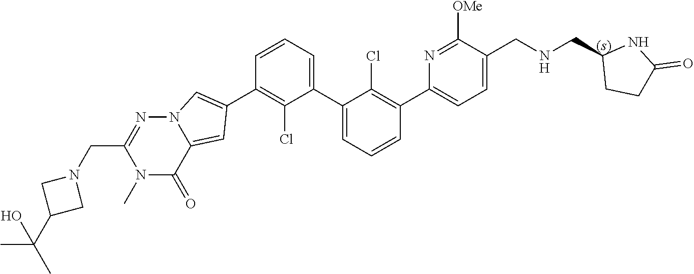 [SMILES: COc1nc(-c2cccc(-c3cccc(-c4cc5c(=O)n(C)c(CN6CC(C(C)(C)O)C6)nn5c4)c3Cl)c2Cl)ccc1CNC[C@@H]1CCC(=O)N1]

### tert-butyl N-[[6-[2-Chloro-3-[2-chloro-3-[2-[[3-(1-hydroxy-1-methyl-ethyl)azetidin-1-yl]methyl]-3-methyl-4-oxo-pyrrolo[2,1-f][1,2,4]triazin-6-yl]phenyl]phenyl]-2-methoxy-3-pyridyl]methyl]-N-[[(2S)-5-oxopyrrolidin-2-yl]methyl]carbamate

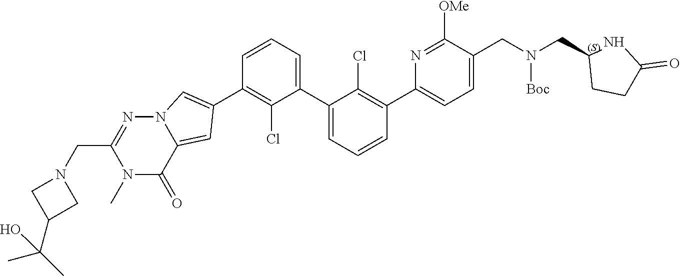

To a solution of tert-butyl N-[[6-[2-chloro-3-[2-chloro-3-[2-(hydroxymethyl)-3-methyl-4-oxo-pyrrolo[2,1-f][1,2,4]triazin-6-yl]phenyl]phenyl]-2-methoxy-3-pyridyl]methyl]-N-[[(2S)-5-oxopyrrolidin-2-yl]methyl]carbamate (20 mg, 0.03 mmol) in DCM (0.1 mL) was added mesyl chloride (2.3 uL, 0.03 mmol) and triethylamine (7.6 uL, 0.05 mmol). The reaction was stirred at rt for 1 h. The solvent was evaporated, and the residue was suspended in DMF (0.4 mL). To this suspension, potassium carbonate (10 mg, 0.07 mmol) and 2-(azetidin-3-yl)propan-2-ol (8.5 mg, 0.07 mmol) were added and the reaction was stirred at rt for 12 h. The solvent was evaporated, and the residue was suspended in water. The solid formed was collected by filtration to afford tert-butyl N-[[6-[2-chloro-3-[2-chloro-3-[2-[[3-(1-hydroxy-1-methyl-ethyl)azetidin-1-yl]methyl]-3-methyl-4-oxo-pyrrolo[2,1-f][1,2,4]triazin-6-yl]phenyl]phenyl]-2-methoxy-3-pyridyl]methyl]-N-[[(2S)-5-oxopyrrolidin-2-yl]methyl]carbamate (18 mg, 87.9%). The material was used as is without any purification. m/z: 830.4 [M+H]⁺ observed.

### 6-[2-Chloro-3-[2-chloro-3-[6-methoxy-5-[[[(2S)-5-oxopyrrolidin-2-yl]methylamino]methyl]-2-pyridyl]phenyl]phenyl]-2-[[3-(1-hydroxy-1-methyl-ethyl)azetidin-1-yl]methyl]-3-methyl-pyrrolo[2,1-f][1,2,4]triazin-4-one

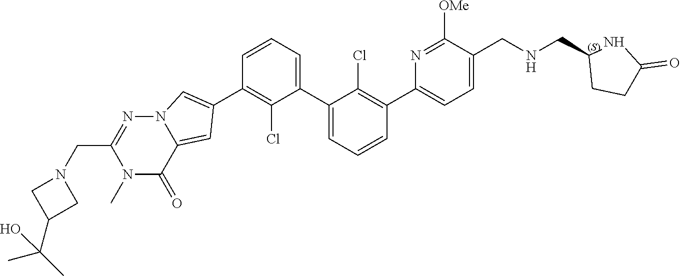

To a solution of tert-butyl N-[[6-[2-chloro-3-[2-chloro-3-[2-[[3-(1-hydroxy-1-methyl-ethyl)azetidin-1-yl]methyl]-3-methyl-4-oxo-pyrrolo[2,1-f][1,2,4]triazin-6-yl]phenyl]phenyl]-2-methoxy-3-pyridyl]methyl]-N-[[(2S)-5-oxopyrrolidin-2-yl]methyl]carbamate (18 mg, 0.02 mmol) in DCM (0.4 mL) was added TFA (83 uL, 1.08 mmol). The reaction was stirred at rt for 30 min. The solvent was evaporated and the residue was purified by reverse phase HPLC (acetonitrile:water) to afford 6-[2-chloro-3-[2-chloro-3-[6-methoxy-5-[[[(2S)-5-oxopyrrolidin-2-yl]methylamino]methyl]-2-pyridyl]phenyl]phenyl]-2-[[3-(1-hydroxy-1-methyl-ethyl)azetidin-1-yl]methyl]-3-methyl-pyrrolo[2,1-f][1,2,4]triazin-4-one (6 mg, 37.9%). m/z: 730.3 [M+H]⁺ observed. ¹H NMR (400 MHz, Methanol-d₄) δ 7.88 (s, 1H), 7.79 (d, J=7.5 Hz, 1H), 7.63 (d, J=7.6 Hz, 2H), 7.47 (dt, J=16.1, 7.7 Hz, 2H), 7.36 (d, J=7.7 Hz, 1H), 7.29 (s, 3H), 4.23 (s, 2H), 4.05 (s, 3H), 4.02-3.92 (m, 5H), 3.83 (s, 3H), 3.51 (s, 3H), 2.90 (s, 2H), 2.79 (t, J=8.0 Hz, 1H), 2.40-2.29 (m, 3H), 1.85 (s, 1H), 1.15 (s, 6H).

### Example 468: 6-[2-Chloro-3-[2-chloro-3-[6-methoxy-5-[[[(2S)-5-oxopyrrolidin-2-yl]methylamino]methyl]-2-pyridyl]phenyl]phenyl]-2-[(6-hydroxy-2-azaspiro[3.3]heptan-2-yl)methyl]-3-methyl-pyrrolo[2,1-f][1,2,4]triazin-4-one

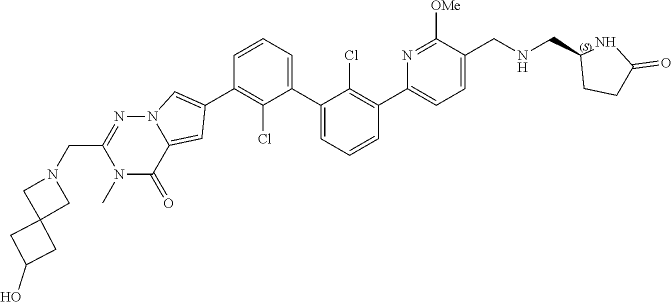

### tert-Butyl N-[[6-[2-chloro-3-[2-chloro-3-[2-[(6-hydroxy-2-azaspiro[3.3]heptan-2-yl)methyl]-3-methyl-4-oxo-pyrrolo[2,1-f][1,2,4]triazin-6-yl]phenyl]phenyl]-2-methoxy-3-pyridyl]methyl]-N-[[(2S)-5-oxopyrrolidin-2-yl]methyl]carbamate

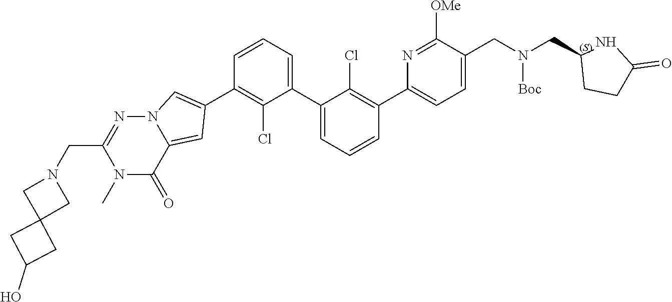

To a solution of tert-butyl N-[[6-[2-chloro-3-[2-chloro-3-[2-(hydroxymethyl)-3-methyl-4-oxo-pyrrolo[2,1-f][1,2,4]triazin-6-yl]phenyl]phenyl]-2-methoxy-3-pyridyl]methyl]-N-[[(2S)-5-oxopyrrolidin-2-yl]methyl]carbamate (20 mg, 0.03 mmol) in DCM (0.1 mL) was added mesyl chloride (2.3 uL, 0.03 mmol) and triethylamine (7.6 uL, 0.05 mmol). The reaction was stirred at rt for 1 h. The solvent was evaporated, and the residue was suspended in DMF (0.4 mL). To this suspension, potassium carbonate (10 mg, 0.07 mmol) and 2-azaspiro[3.3]heptan-6-ol, HCl (11 mg, 0.07 mmol) was added The reaction was heated to 60° C. for 3h. The solvent was evaporated, and the residue was suspended in water. The solid formed was collected by filtration to afford tert-butyl N-[[6-[2-chloro-3-[2-chloro-3-[2-[(6-hydroxy-2-azaspiro[3.3]heptan-2-yl)methyl]-3-methyl-4-oxo-pyrrolo[2,1-f][1,2,4]triazin-6-yl]phenyl]phenyl]-2-methoxy-3-pyridyl]methyl]-N-[[(2S)-5-oxopyrrolidin-2-yl]methyl]carbamate (16 mg, 78%). The material was used as is for the next step. m/z: 830.3 [M+H]⁺ observed.

### 6-[2-Chloro-3-[2-chloro-3-[6-methoxy-5-[[[(2S)-5-oxopyrrolidin-2-yl]methylamino]methyl]-2-pyridyl]phenyl]phenyl]-2-[(6-hydroxy-2-azaspiro[3.3]heptan-2-yl)methyl]-3-methyl-pyrrolo[2,1-f1[1,2,4]triazin-4-one

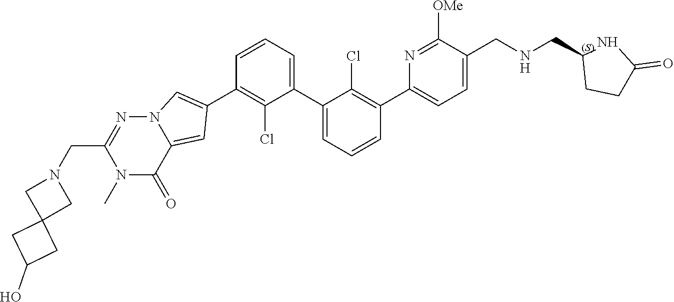

To a solution of tert-butyl N-[[6-[2-chloro-3-[2-chloro-3-[2-[(6-hydroxy-2-azaspiro[3.3]heptan-2-yl)methyl]-3-methyl-4-oxo-pyrrolo[2,1-f][1,2,4]triazin-6-yl]phenyl]phenyl]-2-methoxy-3-pyridyl]methyl]-N-[[(2S)-5-oxopyrrolidin-2-yl]methyl]carbamate (18 mg, 0.02 mmol) in DCM (0.1 mL) was added TFA (33 uL, 0.43 mmol). The reaction was stirred at rt for 1 h. The solvent was evaporated and the residue was purified by reverse phase HPLC (acetonitrile:water) to afford 6-[2-chloro-3-[2-chloro-3-[6-methoxy-5-[[[(2S)-5-oxopyrrolidin-2-yl]methylamino]methyl]-2-pyridyl]phenyl]phenyl]-2-[(6-hydroxy-2-azaspiro[3.3]heptan-2-yl)methyl]-3-methyl-pyrrolo[2,1-f][1,2,4]triazin-4-one (11 mg, 69.5%). m/z: 728.4 [M+H]⁺ observed. ¹H NMR (400 MHz, Methanol-d₄) δ 7.84 (s, 1H), 7.78 (s, 1H), 7.62 (s, 2H), 7.45 (d, J=17.5 Hz, 2H), 7.38-7.33 (m, 1H), 7.27 (s, 3H), 4.10 (s, 1H), 4.04 (d, J=3.3 Hz, 3H), 3.98 (s, 2H), 3.92 (s, 3H), 3.66-3.55 (m, 4H), 3.52 (d, J=3.3 Hz, 3H), 2.86 (s, 2H), 2.53 (s, 2H), 2.34 (s, 3H), 2.06 (s, 2H), 1.84 (s, 1H).

### Example 469: 6-[2-Chloro-3-[2-chloro-3-[6-methoxy-5-[[[(2S)-5-oxopyrrolidin-2-yl]methylamino]methyl]-2-pyridyl]phenyl]phenyl]-2-[(3-hydroxy-3-methyl-azetidin-1-yl)methyl]-3-methyl-pyrrolo[2,1-f][1,2,4]triazin-4-one

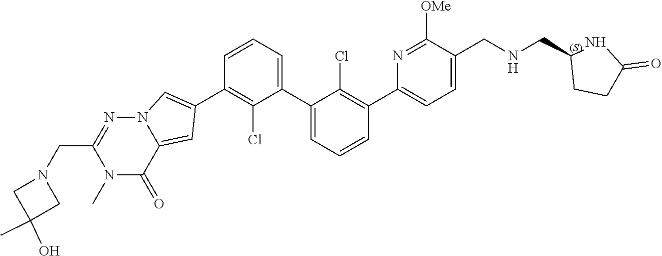

### tert-butyl N-[[6-[2-Chloro-3-[2-chloro-3-[2-[(3-hydroxy-3-methyl-azetidin-1-yl)methyl]-3-methyl-4-oxo-pyrrolo[2,1-f][1,2,4]triazin-6-yl]phenyl]phenyl]-2-methoxy-3-pyridyl]methyl]-N-[[(2S)-5-oxopyrrolidin-2-yl]methyl]carbamate

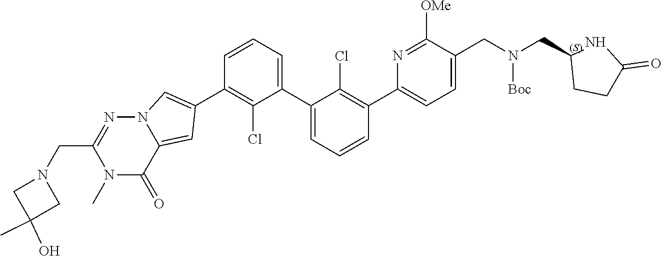

To a solution of tert-butyl N-[[6-[2-chloro-3-[2-chloro-3-[2-(hydroxymethyl)-3-methyl-4-oxo-pyrrolo[2,1-f][1,2,4]triazin-6-yl]phenyl]phenyl]-2-methoxy-3-pyridyl]methyl]-N-[[(2S)-5-oxopyrrolidin-2-yl]methyl]carbamate (20 mg, 0.03 mmol) in DCM (0.4 mL) was added mesyl chloride (2.3 uL, 0.03 mmol) and triethylamine (11 uL, 0.08 mmol). The reaction was stirred at rt for 1 h. The solvent was evaporated, and the residue was taken up in DMF (0.4 mL). Potassium carbonate (11 mg, 0.08 mmol) and 3-methylazetidin-3-ol, HCl (10 mg, 0.08 mmol) was added and the reaction was stirred at rt for 12 h. The solvent was evaporated, and the residue was suspended in water. The solid formed was collected by filtration to afford tert-butyl N-[[6-[2-chloro-3-[2-chloro-3-[2-[(3-hydroxy-3-methyl-azetidin-1-yl)methyl]-3-methyl-4-oxo-pyrrolo[2,1-f][1,2,4]triazin-6-yl]phenyl]phenyl]-2-methoxy-3-pyridyl]methyl]-N-[[(2S)-5-oxopyrrolidin-2-yl]methyl]carbamate (16 mg, 73.1%). The material was used as is without any purification. 804.2 [M+H]⁺ observed.

### 6-[2-Chloro-3-[2-chloro-3-[6-methoxy-5-[[[(2S)-5-oxopyrrolidin-2-yl]methylamino]methyl]-2-pyridyl]phenyl]phenyl]-2-[(3-hydroxy-3-methyl-azetidin-1-yl)methyl]-3-methyl-pyrrolo[2,1-f][1,2,4]triazin-4-one

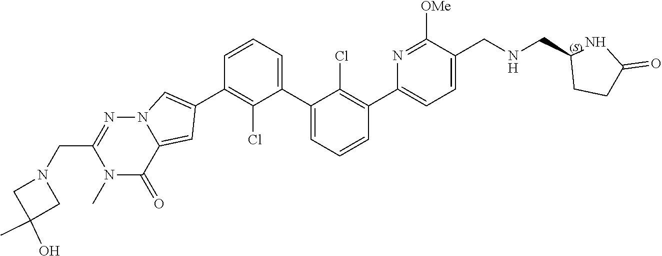

To a solution of tert-butyl N-[[6-[2-chloro-3-[2-chloro-3-[2-[(3-hydroxy-3-methyl-azetidin-1-yl)methyl]-3-methyl-4-oxo-pyrrolo[2,1-f][1,2,4]triazin-6-yl]phenyl]phenyl]-2-methoxy-3-pyridyl]methyl]-N-[[(2S)-5-oxopyrrolidin-2-yl]methyl]carbamate (16 mg, 0.02 mmol) in DCM (0.4 mL) was added TFA (81 uL, 1.06 mmol). The reaction was stirred at rt for 30 min. The solvent was evaporated and the residue was purified by reverse phase HPLC (acetonitrile:water) to afford 6-[2-chloro-3-[2-chloro-3-[6-methoxy-5-[[[(2S)-5-oxopyrrolidin-2-yl]methylamino]methyl]-2-pyridyl]phenyl]phenyl]-2-[(3-hydroxy-3-methyl-azetidin-1-yl)methyl]-3-methyl-pyrrolo[2,1-f][1,2,4]triazin-4-one (7 mg, 47.0%). m/z: 702.4 [M+H]⁺ observed. ¹H NMR (400 MHz, Methanol-d₄): δ 7.83 (d, J=1.9 Hz, 1H), 7.79 (d, J=7.5 Hz, 1H), 7.62 (dt, J=7.8, 2.0 Hz, 2H), 7.46 (dt, J=19.2, 7.7 Hz, 2H), 7.35 (dd, J=7.5, 1.7 Hz, 1H), 7.32-7.23 (m, 3H), 4.05 (s, 4H), 3.97-3.90 (m, 1H), 3.87 (s, 2H), 3.58 (s, 3H), 3.54 (d, J=8.0 Hz, 2H), 2.93 (s, 2H), 2.42-2.26 (m, 3H), 1.86 (d, J=10.4 Hz, 1H), 1.49 (s, 3H).

### Example 470: 6-[2-Chloro-3-[2-chloro-3-[6-methoxy-5-[[[(2S)-5-oxopyrrolidin-2-yl]methylamino]methyl]-2-pyridyl]phenyl]phenyl]-2-[(3-hydroxy-3-methyl-azetidin-1-yl)methyl]-3-methyl-pyrrolo[2,1-f][1,2,4]triazin-4-one

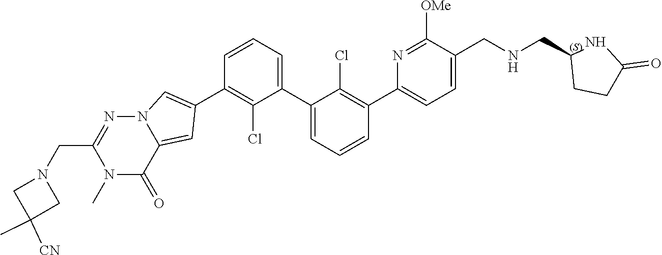

### tert-Butyl N-[[6-[2-chloro-3-[2-chloro-3-[2-[(3-cyano-3-methyl-azetidin-1-yl)methyl]-3-methyl-4-oxo-pyrrolo[2,1-f][1,2,4]triazin-6-yl]phenyl]phenyl]-2-methoxy-3-pyridyl]methyl]-N-[[(2S)-5-oxopyrrolidin-2-yl]methyl]carbamate

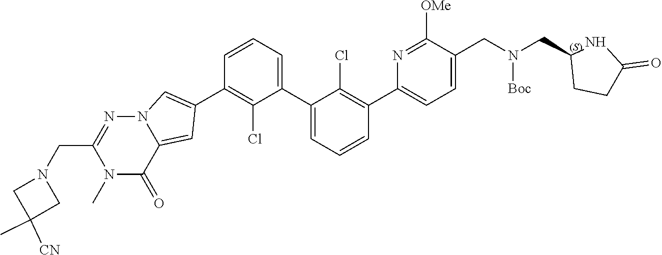

To a solution of tert-butyl N-[[6-[2-chloro-3-[2-chloro-3-[2-(hydroxymethyl)-3-methyl-4-oxo-pyrrolo[2,1-f][1,2,4]triazin-6-yl]phenyl]phenyl]-2-methoxy-3-pyridyl]methyl]-N-[[(2S)-5-oxopyrrolidin-2-yl]methyl]carbamate (20 mg, 0.03 mmol) in DCM (0.4 mL) was added triethylamine (11 uL, 0.08 mmol) and mesyl chloride (2.5 uL, 0.03 mmol). The reaction was stirred at rt for 1 h. The solvent was evaporated, and the residue was taken up in DMF (0.4 mL). Potassium carbonate (11 mg, 0.08 mmol) and 3-methylazetidine-3-carbonitrile, HCl (10 mg, 0.08 mmol) was added and the reaction was stirred at rt for 12 h. The solvent was evaporated, and the residue was suspended in water. The solid formed was collected by filtration to afford tert-butyl N-[[6-[2-chloro-3-[2-chloro-3-[2-[(3-cyano-3-methyl-azetidin-1-yl)methyl]-3-methyl-4-oxo-pyrrolo[2,1-f][1,2,4]triazin-6-yl]phenyl]phenyl]-2-methoxy-3-pyridyl]methyl]-N-[[(2S)-5-oxopyrrolidin-2-yl]methyl]carbamate (16 mg, 72.3%). The material was used as is without any purification. m/z: 811.3 [M+H]⁺ observed.

### 1-[[6-[2-Chloro-3-[2-chloro-3-[6-methoxy-5-[[[(2S)-5-oxopyrrolidin-2-yl]methylamino]methyl]-2-pyridyl]phenyl]phenyl]-3-methyl-4-oxo-pyrrolo[2,1-f]1[1,2,4]triazin-2-yl]methyl]-3-methyl-azetidine-3-carbonitrile

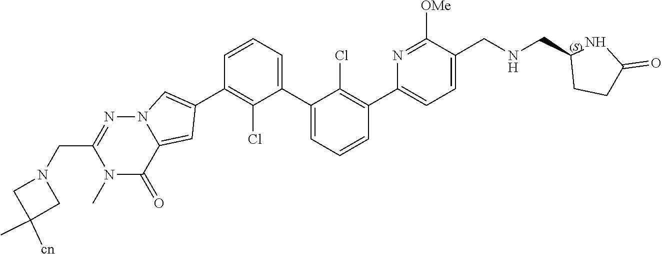

To a solution of tert-butyl N-[[6-[2-chloro-3-[2-chloro-3-[2-[(3-cyano-3-methyl-azetidin-1-yl)methyl]-3-methyl-4-oxo-pyrrolo[2,1-f][1,2,4]triazin-6-yl]phenyl]phenyl]-2-methoxy-3-pyridyl]methyl]-N-[[(2S)-5-oxopyrrolidin-2-yl]methyl]carbamate (16 mg, 0.02 mmol) in DCM (0.4 mL) was added TFA (75 uL, 0.99 mmol). The reaction was stirred at rt for 30 min. The solvent was evaporated and the residue was purified by reverse phase HPLC (acetonitrile:water) to afford 1-[[6-[2-chloro-3-[2-chloro-3-[6-methoxy-5-[[[(2S)-5-oxopyrrolidin-2-yl]methylamino]methyl]-2-pyridyl]phenyl]phenyl]-3-methyl-4-oxo-pyrrolo[2,1-f][1,2,4]triazin-2-yl]methyl]-3-methyl-azetidine-3-carbonitrile (8.6 mg, 61%). m/z: 711.3 [M+H]⁺ observed. ¹H NMR (400 MHz, Methanol-d₄) δ 7.82 (d, J=1.9 Hz, 1H), 7.78 (d, J=7.5 Hz, 1H), 7.62 (ddd, J=7.8, 4.1, 1.7 Hz, 2H), 7.45 (dt, J=19.1, 7.7 Hz, 2H), 7.35 (dd, J=7.6, 1.7 Hz, 1H), 7.31-7.24 (m, 3H), 4.04 (s, 3H), 3.96 (d, J=34.1 Hz, 2H), 3.79 (s, 2H), 3.73 (d, J=7.4 Hz, 2H), 3.58 (s, 3H), 3.40 (d, J=7.4 Hz, 2H), 2.89 (s, 2H), 2.45-2.24 (m, 3H), 1.86 (d, J=10.5 Hz, 1H), 1.63 (s, 3H).

### Example 476: 6-[2-Chloro-3-[2-chloro-3-[6-methoxy-5-[[[(2S)-5-oxopyrrolidin-2-yl]methylamino]methyl]-2-pyridyl]phenyl]phenyl]-3-methyl-2-[[[(3S)-5-oxopyrrolidin-3-yl]methylamino] methyl]pyrrolo[2,1-f][1,2,4]triazin-4-one

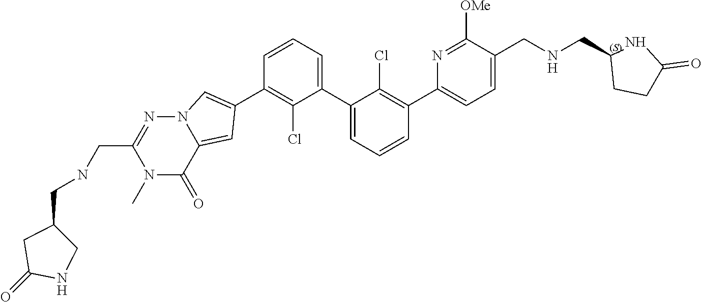

### tert-Butyl N-[[6-[2-chloro-3-[2-chloro-3-[3-methyl-4-oxo-2-[[[(3S)-5-oxopyrrolidin-3-yl]methylamino]methyl]pyrrolo[2,1-f][1,2,4]triazin-6-yl]phenyl]phenyl]-2-methoxy-3-pyridyl]methyl]-N-[[(2S)-5-oxopyrrolidin-2-yl]methyl]carbamate

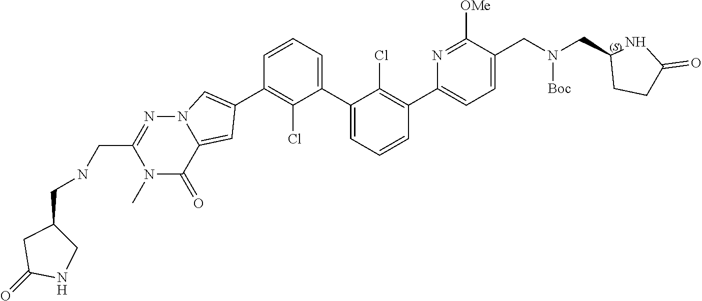

To a solution of tert-butyl N-[[6-[2-chloro-3-[2-chloro-3-[2-(hydroxymethyl)-3-methyl-4-oxo-pyrrolo[2,1-f1][1,2,4]triazin-6-yl]phenyl]phenyl]-2-methoxy-3-pyridyl]methyl]-N-[[(2S)-5-oxopyrrolidin-2-yl]methyl]carbamate (20 mg, 0.03 mmol) in DCM (0.4 mL) was added mesyl chloride (2.5 uL, 0.03 mmol) and triethylamine (11 uL, 0.08 mmol). The reaction was stirred at rt for 1 h. The solvent was evaporated, and the residue was taken up in DMF (0.4 mL). Potassium carbonate (11 mg, 0.08 mmol) and (4S)-4-(aminomethyl)pyrrolidin-2-one (9 mg, 0.08 mmol) were added and the reaction was stirred at rt for 12 h. The solvent was evaporated, and the residue was suspended in water. The solid formed was collected to afford tert-butyl N-[[6-[2-chloro-3-[2-chloro-3-[3-methyl-4-oxo-2-[[[(3S)-5-oxopyrrolidin-3-yl]methylamino]methyl]pyrrolo[2,1-f][1,2,4]triazin-6-yl]phenyl]phenyl]-2-methoxy-3-pyridyl]methyl]-N-[[(2S)-5-oxopyrrolidin-2-yl]methyl]carbamate (16 mg, 70.7%). The material was used as is without any purification. m/z: 829.2 [M+H]⁺ observed.

### 6-[2-Chloro-3-[2-chloro-3-[6-methoxy-5-[[[(2S)-5-oxopyrrolidin-2-yl]methylamino]methyl]-2-pyridyl]phenyl]phenyl]-3-methyl-2-[[[(3S)-5-oxopyrrolidin-3-yl]methylamino]methyl] pyrrolo[2,1-f][1,2,4]triazin-4-one

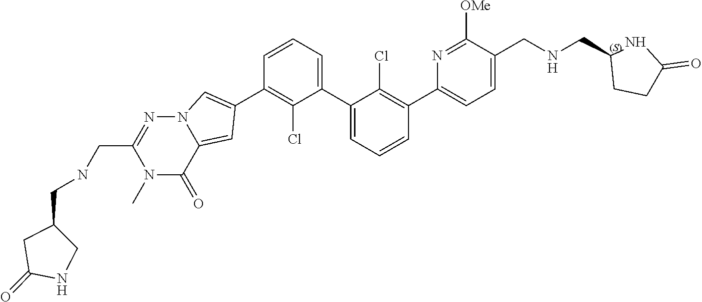

To a solution of tert-butyl N-[[6-[2-chloro-3-[2-chloro-3-[3-methyl-4-oxo-2-[[[(3 S)-5-oxopyrrolidin-3-yl]methylamino]methyl]pyrrolo[2,1-f][1,2,4]triazin-6-yl]phenyl]phenyl]-2-methoxy-3-pyridyl]methyl]-N-[[(2S)-5-oxopyrrolidin-2-yl]methyl]carbamate (16 mg, 0.02 mmol) in DCM (0.4 mL) was added TFA (74 uL, 0.96 mmol). The reaction was stirred at rt for 30 min. The solvent was evaporated and the residue was purified by reverse phase HPLC (acetonitrile:water) to afford 6-[2-chloro-3-[2-chloro-3-[6-methoxy-5-[[[(2S)-5-oxopyrrolidin-2-yl]methylamino]methyl]-2-pyridyl]phenyl]phenyl]-3-methyl-2-[[[(3S)-5-oxopyrrolidin-3-yl]methylamino]methyl]pyrrolo[2,1-f][1,2,4]triazin-4-one (4.80 mg, 34.1%). m/z: 729.3 [M+H]⁺ observed. ¹H NMR (400 MHz, Methanol-d₄): δ 7.84 (s, 1H), 7.77 (d, J=7.5 Hz, 1H), 7.63 (dd, J=7.7, 1.7 Hz, 2H), 7.46 (dt, J=18.0, 7.7 Hz, 2H), 7.35 (dd, J=7.5, 1.7 Hz, 1H), 7.31-7.24 (m, 3H), 4.04 (s, 3H), 3.98 (s, 2H), 3.92 (s, 3H), 3.59 (s, 3H), 3.56-3.49 (m, 1H), 3.18 (dd, J=10.1, 5.3 Hz, 1H), 2.92-2.77 (m, 4H), 2.70 (s, 1H), 2.48 (dd, J=16.9, 8.6 Hz, 1H), 2.40-2.28 (m, 3H), 2.16 (dd, J=16.9, 6.5 Hz, 1H), 1.91-1.77 (m, 1H).

### Example 477: 6-[2-Chloro-3-[2-chloro-3-[6-methoxy-5-[[[(2S)-5-oxopyrrolidin-2-yl]methylamino]methyl]-2-pyridyl]phenyl]phenyl]-2-[(isopropylamino)methyl]-3-methyl-pyrrolo[2,1-f][1,2,4]triazin-4-one

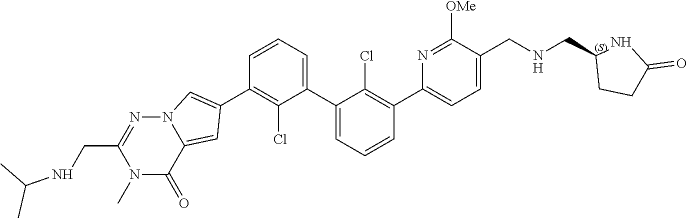

### tert-Butyl N-[[6-[2-chloro-3-[2-chloro-3-[2-[(isopropylamino)methyl]-3-methyl-4-oxo-pyrrolo[2,1-f][1,2,4]triazin-6-yl]phenyl]phenyl]-2-methoxy-3-pyridyl]methyl]-N-[[(2S)-5-oxopyrrolidin-2-yl]methyl]carbamate

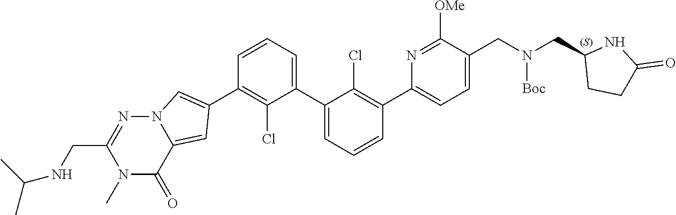

To a solution of tert-butyl N-[[6-[2-chloro-3-[2-chloro-3-[2-(hydroxymethyl)-3-methyl-4-oxo-pyrrolo[2,1-f][1,2,4]triazin-6-yl]phenyl]phenyl]-2-methoxy-3-pyridyl]methyl]-N-[[(2S)-5-oxopyrrolidin-2-yl]methyl]carbamate (20 mg, 0.03 mmol) in DCM (0.4 mL) was added mesyl chloride (2.5 uL, 0.03 mmol) and triethylamine (11 uL, 0.08 mmol). The reaction was stirred at rt for 1 h. The solvent was evaporated, and the residue was taken up in DMF (0.4 mL). Propan-2-amine (22 uL, 0.27 mmol) was added and the reaction was stirred at rt for 12 h. The solvent was evaporated, and the residue was suspended in water. The solid formed was collected to afford tert-butyl N-[[6-[2-chloro-3-[2-chloro-3-[2-[(isopropylamino)methyl]-3-methyl-4-oxo-pyrrolo[2,1-f][1,2,4]triazin-6-yl]phenyl]phenyl]-2-methoxy-3-pyridyl]methyl]-N-[[(2S)-5-oxopyrrolidin-2-yl]methyl]carbamate (16 mg, 75.8%). The material was used as is without any purification. m/z: 776.1 [M+H]⁺ observed.

### 6-[2-Chloro-3-[2-chloro-3-[6-methoxy-5-[[[(2S)-5-oxopyrrolidin-2-yl]methylamino]methyl]-2-pyridyl]phenyl]phenyl]-2-[(isopropylamino)methyl]-3-methyl-pyrrolo[2,1-f][1,2,4]triazin-4-one

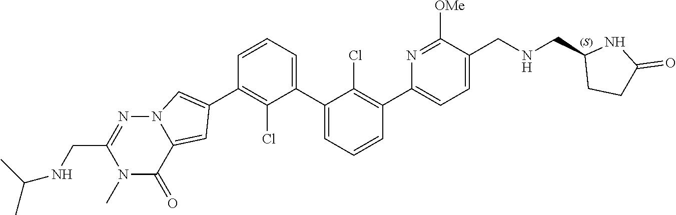

To a solution of tert-butyl N-[[6-[2-chloro-3-[2-chloro-3-[2-[(isopropylamino)methyl]-3-methyl-4-oxo-pyrrolo[2,1-f][1,2,4]triazin-6-yl]phenyl]phenyl]-2-methoxy-3-pyridyl]methyl]-N-[[(2S)-5-oxopyrrolidin-2-yl]methyl]carbamate (16 mg, 0.02 mmol) in DCM (0.4 mL) was added TFA (79 uL, 1.03 mmol). The reaction was stirred at rt for 30 min. The solvent was evaporated and the residue was purified by reverse phase HPLC (acetonitrile:water) to afford 6-[2-chloro-3-[2-chloro-3-[6-methoxy-5-[[[(2S)-5-oxopyrrolidin-2-yl]methylamino]methyl]-2-pyridyl]phenyl] phenyl]-2-[(isopropylamino)methyl]-3-methyl-pyrrolo[2,1-f][1,2,4]triazin-4-one (6.1 mg, 43.8%). m/z: 676.2 [M+H]⁺ observed. ¹H NMR (400 MHz, Methanol-d₄); δ7.90-7.85 (m, 1H), 7.77 (d, J=7.3 Hz, 1H), 7.63 (dd, J=7.8, 1.7 Hz, 2H), 7.46 (dt, J=14.9, 7.6 Hz, 2H), 7.35 (dd, J=7.6, 1.7 Hz, 1H), 7.31-7.25 (m, 3H), 4.23-4.18 (m, 2H), 4.03 (s, 3H), 3.93 (s, 3H), 3.54 (s, 3H), 2.82 (s, 1H), 2.34 (dt, J=15.7, 8.3 Hz, 3H), 1.83 (s, 1H), 1.31 (d, J=6.3 Hz, 6H).

### Example 479: 2-[(tert-Butylamino)methyl]-6-[2-chloro-3-[2-chloro-3-[6-methoxy-5-[[[(2S)-5-oxopyrrolidin-2-yl]methylamino]methyl]-2-pyridyl]phenyl]phenyl]-3-methyl-pyrrolo[2,1-f][1,2,4]triazin-4-one

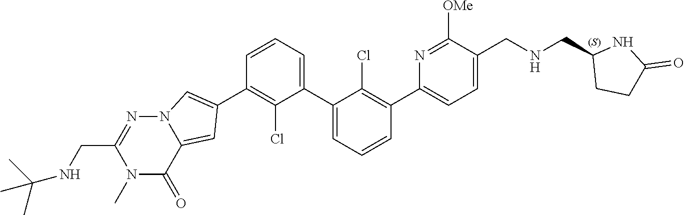

### tert-Butyl N-[[6-[3-[3-[2-[(tert-butylamino)methyl]-3-methyl-4-oxo-pyrrolo[2,1-f][1,2,4]triazin-6-yl]-2-chloro-phenyl]-2-chloro-phenyl]-2-methoxy-3-pyridyl]methyl]-N-[[(2S)-5-oxopyrrolidin-2-yl]methyl]carbamate

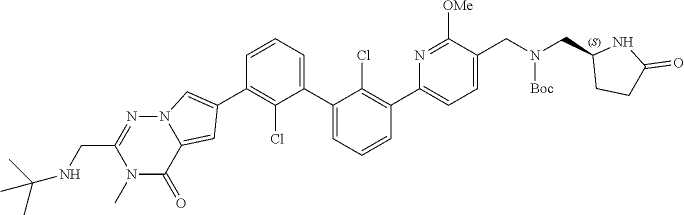

To a solution of tert-butyl N-[[6-[2-chloro-3-[2-chloro-3-[2-(hydroxymethyl)-3-methyl-4-oxo-pyrrolo[2,1-f1][1,2,4]triazin-6-yl]phenyl]phenyl]-2-methoxy-3-pyridyl]methyl]-N-[[(2S)-5-oxopyrrolidin-2-yl]methyl]carbamate (20 mg, 0.03 mmol) in DCM (0.4 mL) was added mesyl chloride (2.5 uL, 0.03 mmol) and triethylamine (11.4 uL, 0.08 mmol). The reaction was stirred at rt for 1 h. The solvent was evaporated, and the residue was taken up in DMF (0.4 mL). 2-methylpropan-2-amine (22 uL, 0.27 mmol) was added and the reaction was stirred at rt for 12 h. The solvent was evaporated, and the residue was suspended in water. The solid formed was collected to afford tert-butyl N-[[6-[3-[3-[2-[(tert-butylamino)methyl]-3-methyl-4-oxo-pyrrolo[2,1-f][1,2,4]triazin-6-yl]-2-chloro-phenyl]-2-chloro-phenyl]-2-methoxy-3-pyridyl]methyl]-N-[[(2S)-5-oxopyrrolidin-2-yl]methyl]carbamate (16 mg, 74.4%). The material was used as is without any purification. m/z: 790.1 [M+H]⁺ observed.

### 2-[(tert-Butylamino)methyl]-6-[2-chloro-3-[2-chloro-3-[6-methoxy-5-[[[(2S)-5-oxopyrrolidin-2-yl]methylamino]methyl]-2-pyridyl]phenyl]phenyl]-3-methyl-pyrrolo[2,1-f][1,2,4]triazin-4-one

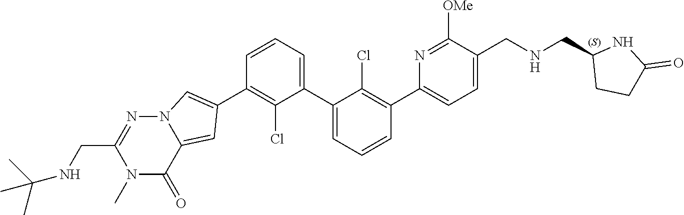

To a solution of tert-butyl N-[[6-[3-[3-[2-[(tert-butylamino)methyl]-3-methyl-4-oxo-pyrrolo[2,1-f][1,2,4]triazin-6-yl]-2-chloro-phenyl]-2-chloro-phenyl]-2-methoxy-3-pyridyl]methyl]-N-[[(2S)-5-oxopyrrolidin-2-yl]methyl]carbamate, (16 mg, 0.02 mmol) in DCM (0.4 mL) was added TFA (78 uL, 1.01 mmol). The reaction was stirred at rt for 30 min. The solvent was evaporated and the residue was purified by reverse phase HPLC (acetonitrile:water) to afford 2-[(tert-butylamino)methyl]-6-[2-chloro-3-[2-chloro-3-[6-methoxy-5-[[[(2S)-5-oxopyrrolidin-2-yl]methylamino]methyl]-2-pyridyl]phenyl]phenyl]-3-methyl-pyrrolo[2,1-f][1,2,4]triazin-4-one (6.7 mg, 48.0%). m/z: 688.3 [M+H]⁺ observed. ¹H NMR (400 MHz, Methanol-d₄): δ 7.89 (d, J=1.8 Hz, 1H), 7.79-7.70 (m, 1H), 7.63 (d, J=7.7 Hz, 2H), 7.46 (dt, J=13.8, 7.7 Hz, 2H), 7.34 (dd, J=7.6, 1.7 Hz, 1H), 7.31-7.24 (m, 3H), 4.16 (s, 2H), 4.03 (s, 3H), 3.89 (s, 2H), 3.57 (s, 3H), 2.78 (s, 1H), 2.32 (q, J=8.8 Hz, 3H), 1.83 (s, 1H), 1.35 (s, 9H).

### Example 482: Methyl 2-[[6-[2-chloro-3-[2-chloro-3-[6-methoxy-5-[[[(2S)-5-oxopyrrolidin-2-yl]methylamino]methyl]-2-pyridyl]phenyl]phenyl]-3-methyl-4-oxo-pyrrolo[2,1-f][1,2,4]triazin-2-yl]methylamino]acetate

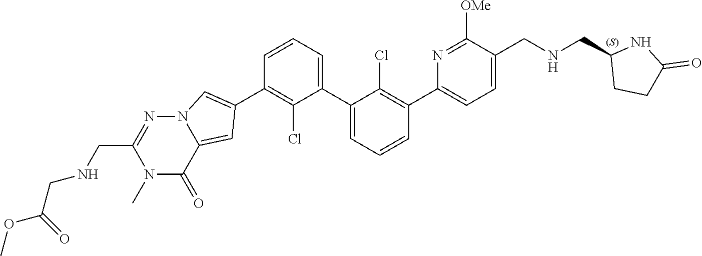

### Methyl 2-[[6-[3-[3-[5-[[tert-butoxycarbonyl-[[(2S)-5-oxopyrrolidin-2-yl]methyl]amino]methyl]-6-methoxy-2-pyridyl]-2-chloro-phenyl]-2-chloro-phenyl]-3-methyl-4-oxo-pyrrolo[2,1-f][1,2,4]triazin-2-yl]methylamino]acetate

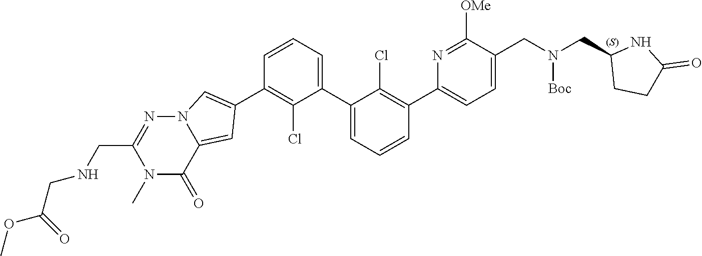

To a solution of tert-butyl N-[[6-[2-chloro-3-[2-chloro-3-[2-(hydroxymethyl)-3-methyl-4-oxo-pyrrolo[2,1-f][1,2,4]triazin-6-yl]phenyl]phenyl]-2-methoxy-3-pyridyl]methyl]-N-[[(2S)-5-oxopyrrolidin-2-yl]methyl]carbamate (20 mg, 0.03 mmol) in DCM (0.4 mL) was added mesyl chloride (2.5 uL, 0.03 mmol) and triethylamine (11 uL, 0.08 mmol). The reaction was stirred at rt for 1 h. The solvent was evaporated, and the residue was taken up in DMF (0.4 mL). Potassium carbonate (11.3 mg, 0.08 mmol) and methyl 2-aminoacetate, HCl (10.3 mg, 0.08 mmol) was added and the reaction was stirred at rt for 12 h. The solvent was evaporated, and the residue was suspended in water. The solid formed was collected by filtration to afford methyl 2-[[6-[3-[3-[5-[[tert-butoxycarbonyl-[[(2S)-5-oxopyrrolidin-2-yl]methyl]amino]methyl]-6-methoxy-2-pyridyl]-2-chloro-phenyl]-2-chloro-phenyl]-3-methyl-4-oxo-pyrrolo[2,1-f][1,2,4]triazin-2-yl]methylamino]acetate (18 mg, 72.9%). The material was used as is without any purification. m/z: 804.3 [M+H]⁺ observed.

### Methyl 2-[[6-[2-chloro-3-[2-chloro-3-[6-methoxy-5-[[[(2S)-5-oxopyrrolidin-2-yl]methylamino]methyl]-2-pyridyl]phenyl]phenyl]-3-methyl-4-oxo-pyrrolo[2,1-f][1,2,4]triazin-2-yl]methylamino]acetate

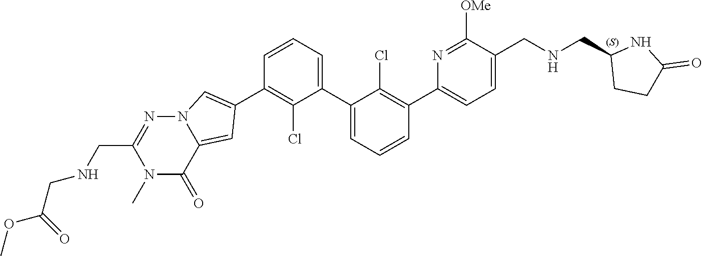

To a solution of methyl 2-[[6-[3-[3-[5-[[tert-butoxycarbonyl-[[(2S)-5-oxopyrrolidin-2-yl]methyl]amino]methyl]-6-methoxy-2-pyridyl]-2-chloro-phenyl]-2-chloro-phenyl]-3-methyl-4-oxo-pyrrolo[2,1-f][1,2,4]triazin-2-yl]methylamino]acetate (12 mg, 0.01 mmol) in DCM (0.4 mL) was added TFA (57 uL, 0.75 mmol). The reaction was stirred at rt for 30 min. The solvent was evaporated and the residue was purified by reverse phase HPLC (acetonitrile:water) to afford methyl 2-[[6-[2-chloro-3-[2-chloro-3-[6-methoxy-5-[[[(2S)-5-oxopyrrolidin-2-yl]methylamino]methyl]-2-pyridyl]phenyl]phenyl]-3-methyl-4-oxo-pyrrolo[2,1-f][1,2,4]triazin-2-yl]methylamino]acetate (4.1 mg, 39.0%). m/z: 704.3 [M+H]⁺ observed. ¹H NMR (400 MHz, Methanol-d₄) δ 7.84 (s, 1H), 7.79 (d, J=7.4 Hz, 1H), 7.63 (dd, J=7.7, 1.7 Hz, 2H), 7.52-7.40 (m, 2H), 7.36 (dd, J=7.6, 1.7 Hz, 1H), 7.32-7.21 (m, 3H), 4.05 (s, 4H), 4.04-3.99 (m, 2H), 3.93 (s, 2H), 3.66 (s, 3H), 3.60 (s, 4H), 2.91 (s, 1H), 2.36 (dd, J=12.1, 4.0 Hz, 3H).

### Example 486: 6-[2-Chloro-3-[2-chloro-3-[6-methoxy-5-[[[(2S)-5-oxopyrrolidin-2-yl]methylamino]methyl]-2-pyridyl]phenyl]phenyl]-2-[[(4-hydroxycyclohexyl)amino]methyl]-3-methyl-pyrrolo[2,1-f][1,2,4]triazin-4-one

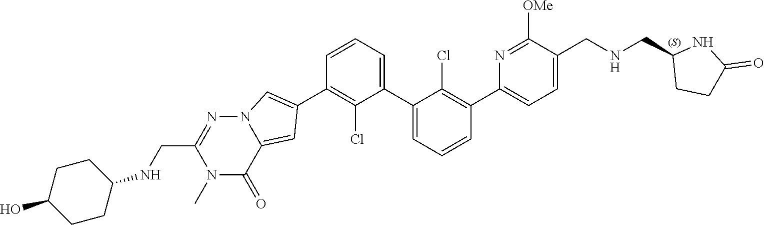

### tert-Butyl N-[[6-[2-chloro-3-[2-chloro-3-[2-[[(4-hydroxycyclohexyl)amino]methyl]-3-methyl-4-oxo-pyrrolo[2,1-f][1,2,4]triazin-6-yl]phenyl]phenyl]-2-methoxy-3-pyridyl]methyl]-N-[[(2S)-5-oxopyrrolidin-2-yl]methyl]carbamate

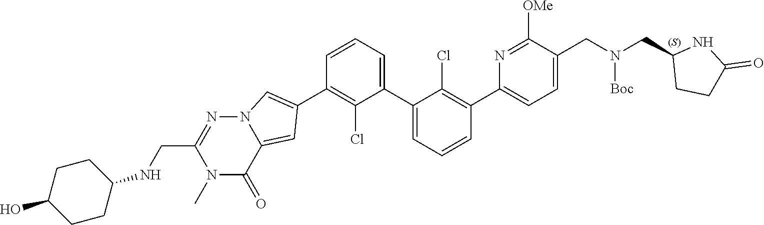

To a solution of tert-butyl N-[[6-[2-chloro-3-[2-chloro-3-[2-(hydroxymethyl)-3-methyl-4-oxo-pyrrolo[2,1-f][1,2,4]triazin-6-yl]phenyl]phenyl]-2-methoxy-3-pyridyl]methyl]-N-[[(2S)-5-oxopyrrolidin-2-yl]methyl]carbamate (15 mg, 0.02 mmol) in DCM (0.4 mL) was added mesyl chloride (2 uL, 0.02 mmol) and triethylamine (8.6 uL, 0.06 mmol). The reaction was stirred at rt for 1 h. The solvent was evaporated, and the residue was taken up in DMF (0.4 mL). Potassium carbonate (85 mg, 0.06 mmol) and 4-aminocyclohexanol, (7 mg, 0.06 mmol) was added and the reaction was stirred at rt for 12 h. The solvent was evaporated, and the residue was suspended in water. The solid formed was collected by filtration to afford tert-butyl N-[[6-[2-chloro-3-[2-chloro-3-[2-[[(4-hydroxycyclohexyl)amino]methyl]-3-methyl-4-oxo-pyrrolo[2,1-f][1,2,4]triazin-6-yl]phenyl]phenyl]-2-methoxy-3-pyridyl]methyl]-N-[[(2S)-5-oxopyrrolidin-2-yl]methyl]carbamate (16 mg, 94.2%). The material was used as is without any purification. m/z: 828.2 [M+H]⁺.

### 6-[2-Chloro-3-[2-chloro-3-[6-methoxy-5-[[[(2S)-5-oxopyrrolidin-2-yl]methylamino]methyl]-2-pyridyl]phenyl]phenyl]-2-[[(4-hydroxycyclohexyl)amino]methyl]-3-methyl-pyrrolo[2,1-f][1,2,4]triazin-4-one

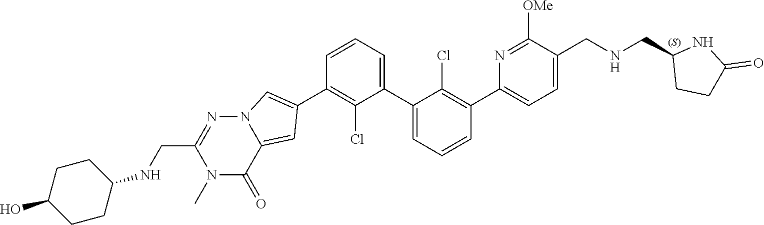

To a solution of tert-butyl N-[[6-[2-chloro-3-[2-chloro-3-[2-[[(4-hydroxycyclohexyl)amino]methyl]-3-methyl-4-oxo-pyrrolo[2,1-f][1,2,4]triazin-6-yl]phenyl]phenyl]-2-methoxy-3-pyridyl]methyl]-N-[[(2S)-5-oxopyrrolidin-2-yl]methyl]carbamate (16 mg, 0.02 mmol) in DCM (0.4 mL) was added TFA (74 uL, 0.96 mmol). The reaction was stirred at rt for 30 min. The solvent was evaporated and the residue was purified by reverse phase HPLC (acetonitrile:water) to afford 6-[2-chloro-3-[2-chloro-3-[6-methoxy-5-[[[(2S)-5-oxopyrrolidin-2-yl]methylamino]methyl]-2-pyridyl]phenyl]phenyl]-2-[[(4-hydroxycyclohexyl)amino]methyl]-3-methyl-pyrrolo[2,1-f][1,2,4]triazin-4-one (7.6 mg, 54.0%). %). m/z: 732.0 [M+H]⁺. ¹H NMR (400 MHz, Methanol-d₄) δ 7.89 (s, 1H), 7.82 (d, J=7.5 Hz, 1H), 7.63 (dd, J=7.8, 1.7 Hz, 2H), 7.47 (dt, J=17.5, 7.6 Hz, 2H), 7.36 (dd, J=7.6, 1.7 Hz, 1H), 7.29 (ddd, J=13.0, 6.1, 2.4 Hz, 3H), 4.27 (s, 2H), 4.10 (s, 2H), 4.06 (s, 3H), 3.96 (s, 1H), 3.62-3.55 (m, 1H), 3.53 (s, 3H), 2.99 (s, 3H), 2.35 (s, 3H), 2.18 (d, J=11.6 Hz, 2H), 2.04 (d, J=11.8 Hz, 2H), 1.86 (s, 1H), 1.48-1.26 (m, 4H).

### Example 487: 2-[[6-[2-Chloro-3-[2-chloro-3-[6-methoxy-5-[[[(2S)-5-oxopyrrolidin-2-yl]methylamino] methyl]-2-pyridyl]phenyl]phenyl]-3-methyl-4-oxo-pyrrolo[2,1-f][1,2,4]triazin-2-yl]methylamino]acetic acid

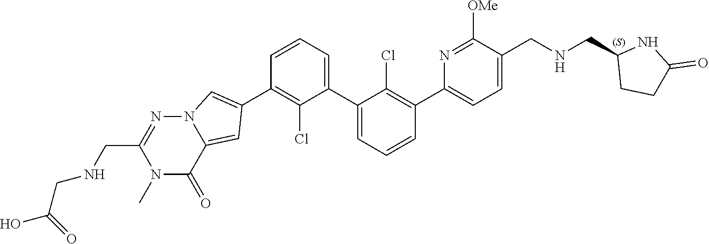

### tert-Butyl 2-[[6-[3-[3-[5-[[tert-butoxycarbonyl-[[(2S)-5-oxopyrrolidin-2-yl]methyl]amino]methyl]-6-methoxy-2-pyridyl]-2-chloro-phenyl]-2-chloro-phenyl]-3-methyl-4-oxo-pyrrolo[2,1-f][1,2,4]triazin-2-yl]methylamino]acetate

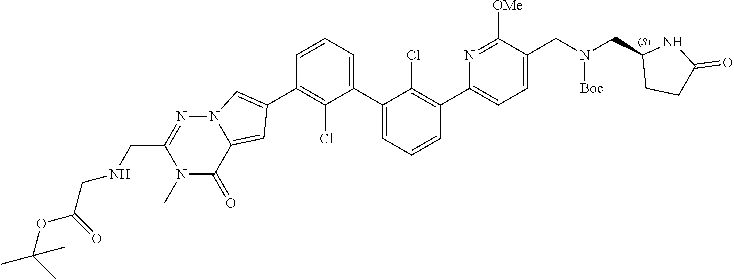

To a solution of tert-butyl N-[[6-[2-chloro-3-[2-chloro-3-[2-(hydroxymethyl)-3-methyl-4-oxo-pyrrolo[2,1-f1][1,2,4]triazin-6-yl]phenyl]phenyl]-2-methoxy-3-pyridyl]methyl]-N-[[(2S)-5-oxopyrrolidin-2-yl]methyl]carbamate (20 mg, 0.03 mmol) in DCM (0.4 mL) was added mesyl chloride (2.5 uL, 0.03 mmol) and triethylamine (11.4 uL, 0.08 mmol). The reaction was stirred at rt for 1 h. The solvent was evaporated, and the residue was taken up in DMF (0.4 mL). Potassium carbonate (11 mg, 0.08 mmol) and tert-butyl 2-aminoacetate (11 mg, 0.08 mmol) were added and the reaction was stirred at rt for 12 h. The solvent was evaporated, and the residue was suspended in water. The solid formed was collected by filtration to afford tert-butyl 2-[[6-[3-[3-[5-[[tert-butoxycarbonyl-[[(2S)-5-oxopyrrolidin-2-yl]methyl]amino]methyl]-6-methoxy-2-pyridyl]-2-chloro-phenyl]-2-chloro-phenyl]-3-methyl-4-oxo-pyrrolo[2,1-f][1,2,4]triazin-2-yl]methylamino]acetate (16 mg, 69.3%). The material was used as is without any purification. m/z: 846.1 [M+H]⁺.

### 2-[[6-[2-Chloro-3-[2-chloro-3-[6-methoxy-5-[[[(2S)-5-oxopyrrolidin-2-yl]methylamino]methyl]-2-pyridyl]phenyl]phenyl]-3-methyl-4-oxo-pyrrolo[2,1-f]1[1,2,4]triazin-2-yl]methylamino]acetic acid

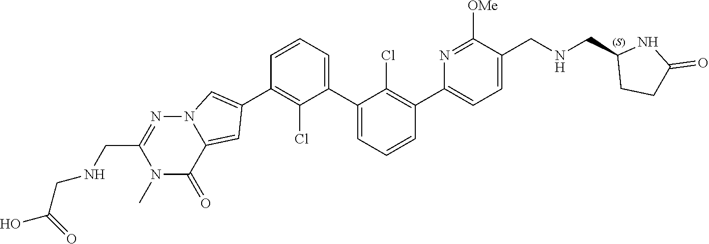

To a solution of tert-butyl 2-[[6-[3-[3-[5-[[tert-butoxycarbonyl-[[(2S)-5-oxopyrrolidin-2-yl]methyl]amino]methyl]-6-methoxy-2-pyridyl]-2-chloro-phenyl]-2-chloro-phenyl]-3-methyl-4-oxo-pyrrolo[2,1-f][1,2,4]triazin-2-yl]methylamino]acetate (15 mg, 0.02 mmol) in DCM (0.4 mL) was added TFA (68 uL, 0.89 mmol). The reaction was stirred at rt for 30 min. The solvent was evaporated and the residue was purified by reverse phase HPLC (acetonitrile:water) to afford 2-[[6-[2-chloro-3-[2-chloro-3-[6-methoxy-5-[[[(2S)-5-oxopyrrolidin-2-yl]methylamino] methyl]-2-pyridyl]phenyl]phenyl]-3-methyl-4-oxo-pyrrolo[2,1-f][1,2,4]triazin-2-yl]methylamino]acetic acid (8 mg, 65.4%). m/z: 690.1 [M+H]⁺. ¹H NMR (400 MHz, Methanol-d₄): δ 7.93 (s, 1H), 7.85 (d, J=7.5 Hz, 1H), 7.63 (d, J=7.7 Hz, 2H), 7.53-7.40 (m, 2H), 7.40-7.24 (m, 4H), 4.43 (s, 2H), 4.22 (s, 2H), 4.08 (s, 3H), 4.01 (s, 1H), 3.67 (s, 2H), 3.49 (s, 3H), 3.12 (t, J=5.7 Hz, 2H), 2.43-2.32 (m, 3H), 1.88 (s, 1H).

### Example 495: Methyl 3-[[6-[2-chloro-3-[2-chloro-3-[6-methoxy-5-[[[(2S)-5-oxopyrrolidin-2-yl] methyl amino]methyl]-2-pyridyl]phenyl]phenyl]-3-methyl-4-oxo-pyrrolo[2,1-f][1,2,4]triazin-2-yl]methylamino]-2,2-dimethyl-propanoate

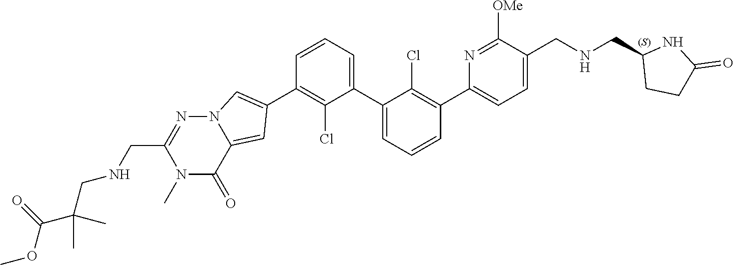

### Methyl 3-[[6-[3-[3-[5-[[tert-butoxycarbonyl-[[(2S)-5-oxopyrrolidin-2-yl]methyl]amino]methyl]-6-methoxy-2-pyridyl]-2-chloro-phenyl]-2-chloro-phenyl]-3-methyl-4-oxo-pyrrolo [2,1-f][1,2,4]triazin-2-yl]methylamino]-2,2-dimethyl-propanoate

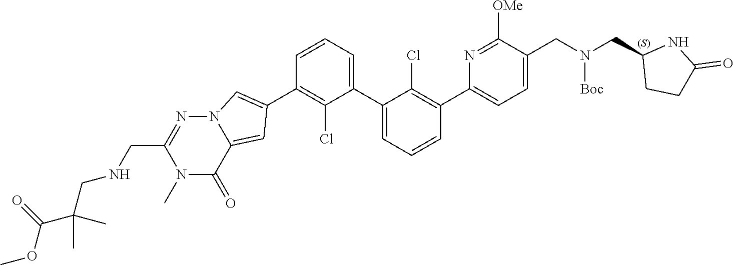

To a solution of tert-butyl N-[[6-[2-chloro-3-[2-chloro-3-[2-(hydroxymethyl)-3-methyl-4-oxo-pyrrolo[2,1-f][1,2,4]triazin-6-yl]phenyl]phenyl]-2-methoxy-3-pyridyl]methyl]-N-[[(2S)-5-oxopyrrolidin-2-yl]methyl]carbamate (20 mg, 0.03 mmol) in DCM (0.4 mL) was added mesyl chloride (2.5 uL, 0.03 mmol) and triethylamine (11.4 uL, 0.08 mmol). The reaction was stirred at rt for 1 h. The solvent was evaporated, and the residue was taken up in DMF (0.4 mL). Potassium carbonate (11.3 mg, 0.08 mmol) and methyl 3-amino-2,2-dimethyl-propanoate, HCl (14 mg, 0.08 mmol) were added and the reaction was stirred at rt for 12 h. The solvent was evaporated, and the residue was suspended in water. The solid formed was collected by filtration to afford methyl 3-[[6-[3-[3-[5-[[tert-butoxycarbonyl-[[(2S)-5-oxopyrrolidin-2-yl]methyl]amino]methyl]-6-methoxy-2-pyridyl]-2-chloro-phenyl]-2-chloro-phenyl]-3-methyl-4-oxo-pyrrolo[2,1-f][1,2,4]triazin-2-yl]methylamino]-2,2-dimethyl-propanoate (17 mg, 73.6%). m/z: 848.1 [M+H]⁺. The material was used as is without any purification.

### Methyl 3-[[6-[2-chloro-3-[2-chloro-3-[6-methoxy-5-[[[(2S)-5-oxopyrrolidin-2-yl] methyl amino]methyl]-2-pyridyl]phenyl]phenyl]-3-methyl-4-oxo-pyrrolo[2,1-f][1,2,4]triazin-2-yl]methylamino]-2,2-dimethyl-propanoate

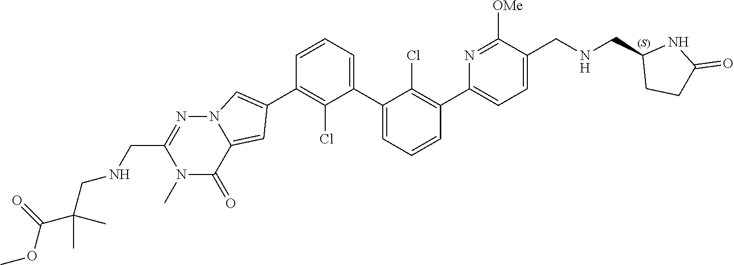

To a solution of methyl 3-[[6-[3-[3-[5-[[tert-butoxycarbonyl-[[(2S)-5-oxopyrrolidin-2-yl]methyl]amino]methyl]-6-methoxy-2-pyridyl]-2-chloro-phenyl]-2-chloro-phenyl]-3-methyl-4-oxo-pyrrolo[2,1-f][1,2,4]triazin-2-yl]methylamino]-2,2-dimethyl-propanoate (17 mg, 0.02 mmol) in DCM (0.1 mL) was added TFA (31 uL, 0.40 mmol). The reaction was stirred at rt for 1 h. The solvent was evaporated and the residue was purified by reverse phase HPLC (acetonitrile:water) to afford methyl 3-[[6-[2-chloro-3-[2-chloro-3-[6-methoxy-5-[[[(2S)-5-oxopyrrolidin-2-yl]methylamino]methyl]-2-pyridyl]phenyl]phenyl]-3-methyl-4-oxo-pyrrolo[2,1-f][1,2,4]triazin-2-yl]methylamino]-2,2-dimethyl-propanoate (7.9 mg, 52.7%). m/z: 747.2 [M+H]⁺. ¹H NMR (400 MHz, Methanol-d₄): δ 7.82 (d, J=1.8 Hz, 1H), 7.74 (d, J=7.5 Hz, 1H), 7.62 (ddd, J=7.7, 3.5, 1.7 Hz, 2H), 7.45 (dt, J=15.7, 7.6 Hz, 2H), 7.34 (dd, J=7.6, 1.7 Hz, 1H), 7.28-7.23 (m, 3H), 4.02 (s, 3H), 3.88 (d, J=3.9 Hz, 2H), 3.84 (s, 3H), 3.58 (d, J=4.4 Hz, 6H), 2.80 (s, 2H), 2.76 (t, J=6.5 Hz, 2H), 2.41-2.22 (m, 3H), 1.16 (s, 6H).

### Example 503: 3-[[6-[2-Chloro-3-[2-chloro-3-[6-methoxy-5-[[[(2S)-5-oxopyrrolidin-2-yl]methylamino] methyl]-2-pyridyl]phenyl]phenyl]-3-methyl-4-oxo-pyrrolo[2,1-f][1,2,4]triazin-2-yl]methylamino]-2,2-dimethyl-propanoic acid

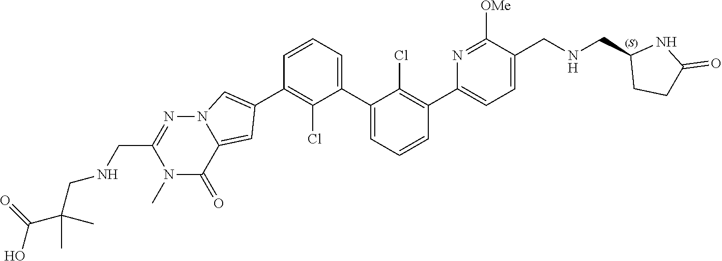

To a solution of methyl 3-[[6-[2-chloro-3-[2-chloro-3-[6-methoxy-5-[[[(2S)-5-oxopyrrolidin-2-yl]methylamino]methyl]-2-pyridyl]phenyl]phenyl]-3-methyl-4-oxo-pyrrolo[2,1-f][1,2,4]triazin-2-yl]methylamino]-2,2-dimethyl-propanoate (6.7 mg, 0.01 mmol) in DCM (0.1 mL) was added lithium hydroxide (0.6 mg, 0.03 mmol). The reaction was stirred at rt for 1 h. The solvent was evaporated and the residue was purified by reverse phase HPLC (acetonitrile:water) to afford 3-[[6-[2-chloro-3-[2-chloro-3-[6-methoxy-5-[[[(2S)-5-oxopyrrolidin-2-yl]methylamino]methyl]-2-pyridyl]phenyl]phenyl]-3-methyl-4-oxo-pyrrolo[2,1-f][1,2,4]triazin-2-yl]methylamino]-2,2-dimethyl-propanoic acid (4.1 mg, 62.4%). m/z: 732.3 [M+H]⁺. ¹H NMR (400 MHz, Methanol-d₄) δ 7.91 (s, 1H), 7.74 (d, J=7.5 Hz, 1H), 7.66-7.57 (m, 2H), 7.46 (dt, J=14.7, 7.6 Hz, 2H), 7.35 (dd, J=7.5, 1.6 Hz, 1H), 7.31-7.22 (m, 3H), 4.20 (s, 2H), 4.02 (s, 3H), 3.86 (d, J=14.0 Hz, 3H), 3.55 (s, 3H), 2.98 (s, 2H), 2.75 (t, J=6.4 Hz, 2H), 2.43-2.24 (m, 3H), 1.23 (s, 6H).

### Example 496: Methyl 1-[[6-[2-chloro-3-[2-chloro-3-[6-methoxy-5-[[[(2S)-5-oxopyrrolidin-2-yl]methyl amino]methyl]-2-pyridyl]phenyl]phenyl]-3-methyl-4-oxo-pyrrolo[2,1-f][1,2,4]triazin-2-yl]methyl]azetidine-3-carboxylate

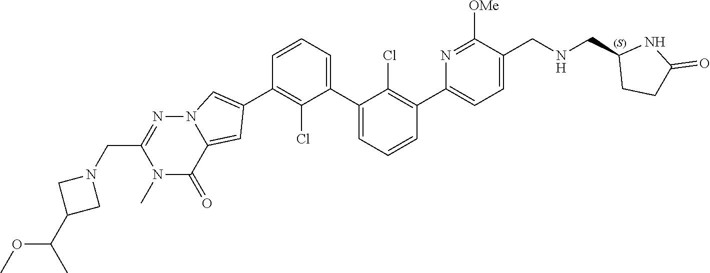

### Methyl 1-[[6-[3-[3-[5-[[tert-butoxycarbonyl-[[(2S)-5-oxopyrrolidin-2-yl]methyl]amino]methyl]-6-methoxy-2-pyridyl]-2-chloro-phenyl]-2-chloro-phenyl]-3-methyl-4-oxo-pyrrolo[2,1-f][1,2,4]triazin-2-yl]methyl]azetidine-3-carboxylate

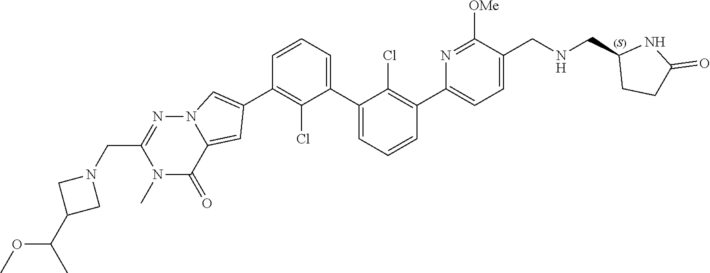

To a solution of tert-butyl N-[[6-[2-chloro-3-[2-chloro-3-[2-(hydroxymethyl)-3-methyl-4-oxo-pyrrolo[2,1-f1][1,2,4]triazin-6-yl]phenyl]phenyl]-2-methoxy-3-pyridyl]methyl]-N-[[(2S)-5-oxopyrrolidin-2-yl]methyl]carbamate (20 mg, 0.03 mmol) in DCM (0.4 mL) was added mesyl chloride (2.5 uL, 0.03 mmol) and triethylamine (11.4 uL, 0.08 mmol). The reaction was stirred at rt for 1 h. The solvent was evaporated, and the residue was taken up in DMF (0.4 mL). Potassium carbonate (11.30 mg, 0.08 mmol) and methyl azetidine-3-carboxylate, HCl (12.4 mg, 0.08 mmol) was added and the reaction was stirred at rt for 12 h. The solvent was evaporated, and the residue was suspended in water. The solid formed was collected by filtration to afford methyl 1-[[6-[3-[3-[5-[[tert-butoxycarbonyl-[[(2S)-5-oxopyrrolidin-2-yl]methyl]amino]methyl]-6-methoxy-2-pyridyl]-2-chloro-phenyl]-2-chloro-phenyl]-3-methyl-4-oxo-pyrrolo[2,1-f][1,2,4]triazin-2-yl]methyl]azetidine-3-carboxylate (18 mg, 79.4%). The material was used as is without any purification. m/z: 832.0 [M+H]⁺.

### Methyl 1-[[6-[2-chloro-3-[2-chloro-3-[6-methoxy-5-[[[(2S)-5-oxopyrrolidin-2-yl]methyl amino]methyl]-2-pyridyl]phenyl]phenyl]-3-methyl-4-oxo-pyrrolo[2,1-f][1,2,4]triazin-2-yl]methyl]azetidine-3-carboxylate

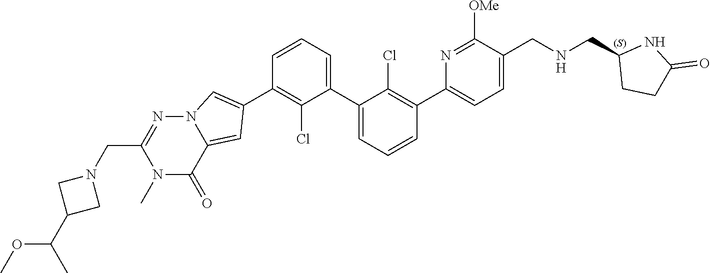

To a solution of methyl 1-[[6-[3-[3-[5-[[tert-butoxycarbonyl-[[(2S)-5-oxopyrrolidin-2-yl]methyl]amino]methyl]-6-methoxy-2-pyridyl]-2-chloro-phenyl]-2-chloro-phenyl]-3-methyl-4-oxo-pyrrolo[2,1-f][1,2,4]triazin-2-yl]methyl]azetidine-3-carboxylate (18 mg, 0.02 mmol) in DCM (0.1 mL) was added TFA (33 uL, 0.43 mmol). The reaction was stirred at rt for 1 h. The solvent was evaporated and the residue was purified by reverse phase HPLC (acetonitrile: water) to afford methyl 1-[[6-[2-chloro-3-[2-chloro-3-[6-methoxy-5-[[[(2S)-5-oxopyrrolidin-2-yl]methylamino]methyl]-2-pyridyl]phenyl]phenyl]-3-methyl-4-oxo-pyrrolo[2,1-f][1,2,4]triazin-2-yl]methyl]azetidine-3-carboxylate (8.7 mg, 55.0%). m/z: 730.3 [M+H]⁺. ¹H NMR (400 MHz, Methanol-d₄) δ 7.82 (d, J=1.9 Hz, 1H), 7.73 (d, J=7.5 Hz, 1H), 7.62 (dt, J=7.8, 1.8 Hz, 2H), 7.45 (dt, J=16.6, 7.6 Hz, 2H), 7.34 (dd, J=7.7, 1.7 Hz, 1H), 7.25 (dt, J=7.5, 2.7 Hz, 3H), 4.02 (s, 3H), 3.86 (d, J=3.7 Hz, 3H), 3.75 (s, 2H), 3.71 (s, 4H), 3.67 (d, J=7.8 Hz, 2H), 3.56 (s, 3H), 3.52 (t, J=7.2 Hz, 3H), 3.41 (q, J=7.6 Hz, 1H), 2.81-2.67 (m, 2H), 2.38-2.23 (m, 3H).

### Example 505: 1-[[6-[2-Chloro-3-[2-chloro-3-[6-methoxy-5-[[[(2S)-5-oxopyrrolidin-2-yl]methylamino] methyl]-2-pyridyl]phenyl]phenyl]-3-methyl-4-oxo-pyrrolo[2,1-f][1,2,4]triazin-2-yl]methyl]azetidine-3-carboxylic acid

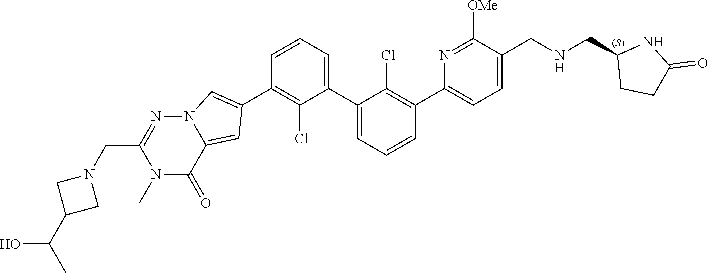

To a solution of methyl 1-[[6-[2-chloro-3-[2-chloro-3-[6-methoxy-5-[[[(2S)-5-oxopyrrolidin-2-yl]methylamino]methyl]-2-pyridyl]phenyl]phenyl]-3-methyl-4-oxo-pyrrolo[2,1-f][1,2,4]triazin-2-yl]methyl]azetidine-3-carboxylate (7.7 mg, 0.01 mmol) in THE (0.4 mL) and water (0.1 mL) was added lithium hydroxide (0.8 mg, 0.03 mmol). The reaction was stirred at rt for 1 h. The solvent was evaporated and the residue was purified by reverse phase HPLC (acetonitrile:water) to afford 1-[[6-[2-chloro-3-[2-chloro-3-[6-methoxy-5-[[[(2S)-5-oxopyrrolidin-2-yl]methylamino]methyl]-2-pyridyl]phenyl]phenyl]-3-methyl-4-oxo-pyrrolo[2,1-f][1,2,4]triazin-2-yl]methyl]azetidine-3-carboxylic acid (3 mg, 39.7%). m/z: 718.0 [M+H]⁺. ¹H NMR (400 MHz, Methanol-d₄): δ 7.87 (d, J=1.8 Hz, 1H), 7.79 (d, J=7.4 Hz, 1H), 7.63 (d, J=7.6 Hz, 2H), 7.46 (dt, J=17.9, 7.6 Hz, 2H), 7.36 (d, J=7.2 Hz, 1H), 7.32-7.24 (m, 3H), 4.19 (s, 2H), 4.05 (s, 8H), 3.92 (d, J=8.5 Hz, 3H), 3.52 (s, 3H), 2.93 (s, 2H), 2.36 (d, J=9.4 Hz, 3H).

### Example 504: (2R)-2-[[6-[2-Chloro-3-[2-chloro-3-[6-methoxy-5-[[[(2S)-5-oxopyrrolidin-2-yl]methylamino] methyl]-2-pyridyl]phenyl]phenyl]-3-methyl-4-oxo-pyrrolo[2,1-f][1,2,4]triazin-2-yl]methylamino]propanoate

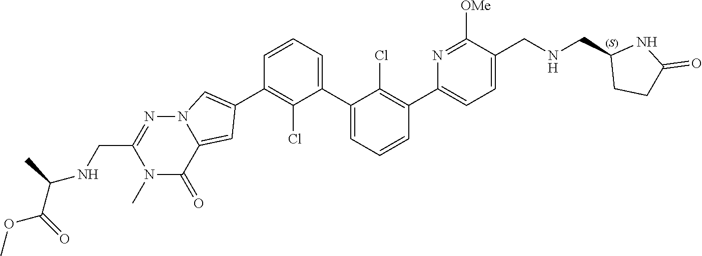

### Methyl (2R)-2-[[6-[3-[3-[5-[[tert-butoxycarbonyl-[[(2S)-5-oxopyrrolidin-2-yl]methyl]amino] methyl]-6-methoxy-2-pyridyl]-2-chloro-phenyl]-2-chloro-phenyl]-3-methyl-4-oxo-pyrrolo[2,1-f][1,2,4]triazin-2-yl]methylamino]propanoate

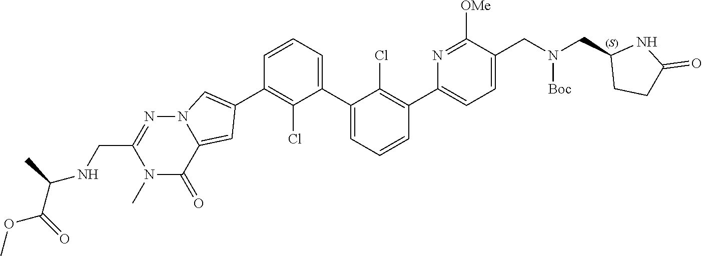

To a solution of tert-butyl N-[[6-[2-chloro-3-[2-chloro-3-[2-(hydroxymethyl)-3-methyl-4-oxo-pyrrolo[2,1-f1][1,2,4]triazin-6-yl]phenyl]phenyl]-2-methoxy-3-pyridyl]methyl]-N-[[(2S)-5-oxopyrrolidin-2-yl]methyl]carbamate (27 mg, 0.04 mmol) in DCM (0.4 mL) was added mesyl chloride (3.4 uL, 0.04 mmol) and triethylamine (15 uL, 0.11 mmol). The reaction was stirred at rt for 1 h. The solvent was evaporated, and the residue was taken up in DMF (0.4 mL). Potassium carbonate (15 mg, 0.11 mmol) and methyl (2R)-2-aminopropanoate, HCl (15.41 mg, 0.11 mmol) were added and the reaction was stirred at rt for 12 h. The solvent was evaporated, and the residue was suspended in water. The solid formed was collected by filtration to afford methyl (2R)-2-[[6-[3-[3-[5-[[tert-butoxycarbonyl-[[(2S)-5-oxopyrrolidin-2-yl]methyl]amino]methyl]-6-methoxy-2-pyridyl]-2-chloro-phenyl]-2-chloro-phenyl]-3-methyl-4-oxo-pyrrolo[2,1-f][1,2,4]triazin-2-yl]methylamino]propanoate (27 mg, 89.6%). The material was used as is without any purification. m/z: 818.0 [M+H]⁺.

### (2R)-2-[[6-[2-Chloro-3-[2-chloro-3-[6-methoxy-5-[[[(2S)-5-oxopyrrolidin-2-yl]methylamino] methyl]-2-pyridyl]phenyl]phenyl]-3-methyl-4-oxo-pyrrolo[2,1-f][1,2,4]triazin-2-yl]methylamino]propanoate

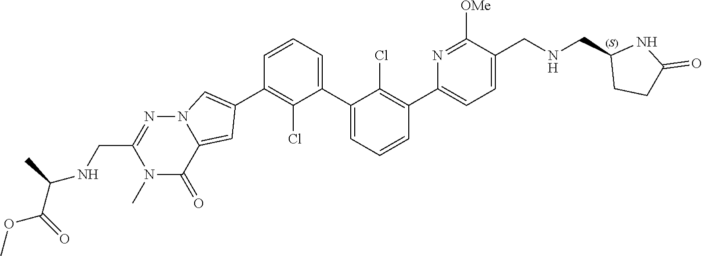

To a solution of methyl (2R)-2-[[6-[3-[3-[5-[[tert-butoxycarbonyl-[[(2S)-5-oxopyrrolidin-2-yl]methyl]amino]methyl]-6-methoxy-2-pyridyl]-2-chloro-phenyl]-2-chloro-phenyl]-3-methyl-4-oxo-pyrrolo[2,1-f][1,2,4]triazin-2-yl]methylamino]propanoate (27 mg, 0.03 mmol) in DCM (0.1 mL) was added TFA (50 uL, 0.66 mmol). The reaction was stirred at rt for 1 h. The solvent was evaporated and the residue was purified by reverse phase HPLC (acetonitrile:water) to afford methyl (2R)-2-[[6-[2-chloro-3-[2-chloro-3-[6-methoxy-5-[[[(2S)-5-oxopyrrolidin-2-yl]methylamino]methyl]-2-pyridyl]phenyl]phenyl]-3-methyl-4-oxo-pyrrolo[2,1-f][1,2,4]triazin-2-yl]methylamino]propanoate (15 mg, 54.9%). m/z: 718.2 [M+H]⁺. ¹H NMR (400 MHz, Methanol-d₄) δ 8.20 (s, 3H), 7.86 (d, J=7.6 Hz, 1H), 7.82 (d, J=1.8 Hz, 1H), 7.66-7.60 (m, 2H), 7.50 (t, J=7.6 Hz, 1H), 7.44 (t, J=7.7 Hz, 1H), 7.40-7.37 (m, 1H), 7.35 (d, J=7.5 Hz, 1H), 7.27 (dt, J=5.2, 2.4 Hz, 2H), 4.25 (d, J=2.1 Hz, 2H), 4.09 (s, 3H), 4.06-3.98 (m, 1H), 3.91 (d, J=1.9 Hz, 2H), 3.62 (d, J=8.6 Hz, 6H), 3.58-3.52 (m, 1H), 3.19-3.11 (m, 2H), 2.41-2.36 (m, 2H), 1.35 (d, J=7.1 Hz, 3H).

### Example 507: (2R)-2-[[6-[2-Chloro-3-[2-chloro-3-[6-methoxy-5-[[[(2S)-5-oxopyrrolidin-2-yl]methylamino] methyl]-2-pyridyl]phenyl]phenyl]-3-methyl-4-oxo-pyrrolo[2,1-f][1,2,4]triazin-2-yl]methylamino]propanoic acid

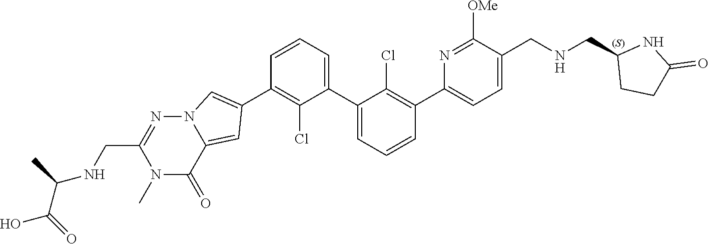

To a solution of methyl (2R)-2-[[6-[3-[3-[5-[[tert-butoxycarbonyl-[[(2S)-5-oxopyrrolidin-2-yl]methyl]amino]methyl]-6-methoxy-2-pyridyl]-2-chloro-phenyl]-2-chloro-phenyl]-3-methyl-4-oxo-pyrrolo[2,1-f][1,2,4]triazin-2-yl]methylamino]propanoate (14 mg, 0.02 mmol) in THE (0.4 mL) and water (0.1 mL) was added lithium hydroxide (2 mg, 0.09 mmol). The reaction was stirred at rt for 12 h. The solvent was evaporated and the residue was purified by reverse phase HPLC (acetonitrile: water) to afford (2R)-2-[[6-[2-chloro-3-[2-chloro-3-[6-methoxy-5-[[[(2S)-5-oxopyrrolidin-2-yl]methylamino]methyl]-2-pyridyl]phenyl]phenyl]-3-methyl-4-oxo-pyrrolo[2,1-f][1,2,4]triazin-2-yl]methylamino]propanoic acid (11.00 mg, 91.3%). m/z: 706.1 [M+H]⁺. ¹H NMR (400 MHz, Methanol-d₄) δ 8.78 (d, J=1.8 Hz, 1H), 8.66 (d, J=7.5 Hz, 1H), 8.52 (dd, J=13.3, 7.8 Hz, 2H), 8.40-8.29 (m, 2H), 8.24 (d, J=7.3 Hz, 1H), 8.18-8.08 (m, 3H), 4.81 (s, 4H), 4.66 (d, J=3.2 Hz, 2H), 4.60 (d, J=8.8 Hz, 1H), 4.39 (s, 3H), 4.25 (q, J=6.9 Hz, 1H), 4.03 (p, J=1.6 Hz, 3H), 3.59-3.45 (m, 2H), 3.06-3.01 (m, 2H), 2.18 (d, J=7.0 Hz, 3H).

### Example 465: 6-(2,2′-Dichloro-3′-(4-chloro-6-methoxy-5-(((((S)-5-oxopyrrolidin-2-yl)methyl)amino) methyl)pyridin-2-yl)-[1,1′-biphenyl]-3-yl)-2-((((1r,3r)-3-hydroxy-3-methylcyclobutyl) amino)methyl)-3-methylpyrrolo[2,1-f][1,2,4]triazin-4(3H)-one

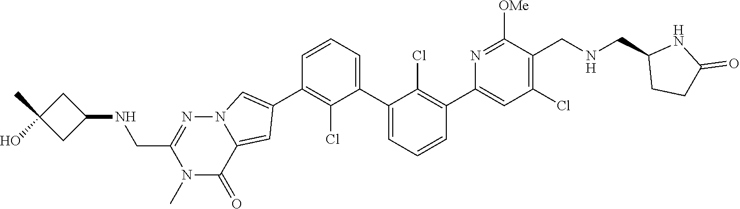

### 6-(3-Bromo-2-chlorophenyl)-4-chloro-2-methoxynicotinaldehyde

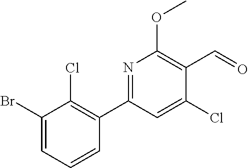 [SMILES: COc1nc(-c2cccc(Br)c2Cl)cc(Cl)c1C=O]

To a mixture of 4,6-dichloro-2-methoxynicotinaldehyde (4.9 g, 23.8 mmol), 2-(3-bromo-2-chlorophenyl)-4,4,5,5-tetramethyl-1,3,2-dioxaborolane (7.17 g, 22.6 mmol) in dioxane/water (5:1, 60 mL) was added cesium carbonate (23.3 g, 71.4 mmol), tetrakis(triphenylphosphine) palladium (1.37 g, 1.19 mmol), and stirred at 100° C. for 2 h. The reaction mixture was diluted with water (50 mL) and extracted with EtOAc (2×200 ml). The combined organic phase was dried with anhydrous sodium sulfate, filtered and concentrated. The residue was triturated with EtOAc/petroleum ether (1/5, 60 mL) at rt for 0.5 h to afford 6-(3-bromo-2-chlorophenyl)-4-chloro-2-methoxynicotinaldehyde (5.3 g, 62% yield) as a white solid. ¹H NMR (400 MHz, DMSO-d₆): δ 10.36 (s, 1H), 7.94 (d, J₁=1.6, J₂=8, Hz, 1H), 7.67 (d, J₁=1.6, J₁=7.6, Hz, 1H), 7.56 (s, 1H), 7.45 (t, J=8 Hz, 1H), 4.02 (s, 3H).

### 4-Chloro-6-(2-chloro-3-(4,4,5,5-tetramethyl-1,3,2-dioxaborolan-2-yl)phenyl)-2-methoxynicotinaldehyde

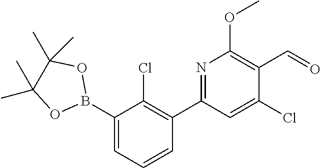 [SMILES: COc1nc(-c2cccc(B3OC(C)(C)C(C)(C)O3)c2Cl)cc(Cl)c1C=O]

To a mixture of 6-(3-bromo-2-chlorophenyl)-4-chloro-2-methoxynicotinaldehyde (1.3 g, 3.6 mmol), 4,4,4′,4′,5,5,5′,5′-octamethyl-2,2′-bi(1,3,2-dioxaborolane) (1.65 g, 6.48 mmol) in dioxane (12 mL) was added potassium acetate (707 mg, 7.2 mmol), [1,1′-bis(di-tert-butylphosphino) ferrocene]dichloropalladium(II) (263 mg, 0.36 mmol). The mixture was stirred at 95° C. for 2 h. The mixture was filtered, and the filtrate was concentrated. The residue was purified by normal phase SiO₂ chromatography (0-8% EtOAc/petroleum ether) to afford 4-chloro-6-(2-chloro-3-(4,4,5,5-tetramethyl-1,3,2-dioxaborolan-2-yl)phenyl)-2-methoxynicotinaldehyde (800 mg, 54% yield) as a white solid. ¹H NMR (400 MHz, DMSO-d₆): δ10.39 (s, 1H), 7.57 (d, J₁=1.2, J₂=7.6, Hz, 2H), 7.52 (t, J=7.6 Hz, 2H), 4.04 (s, 3H), 1.37 (s, 12H).

### 6-(3′-Bromo-2,2′-dichloro-[1,1′-biphenyl]-3-yl)-4-chloro-2-methoxynicotinaldehyde

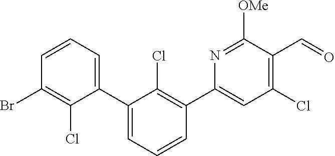 [SMILES: COc1nc(-c2cccc(-c3cccc(Br)c3Cl)c2Cl)cc(Cl)c1C=O]

To a mixture of 4-chloro-6-(2-chloro-3-(4,4,5,5-tetramethyl-1,3,2-dioxaborolan-2-yl)phenyl)-2-methoxynicotinaldehyde (800 mg, 1.96 mmol), 1,3-dibromo-2-chloro-benzene (1.59 g, 5.88 mmol) in dioxane/water (6:1, 21 mL) was added cesium carbonate (1.92 g, 5.88 mmol) and tetrakis(triphenylphosphine)palladium (113 mg, 0.1 mmol). The mixture was stirred at 100° C. for 1.5 h under N₂. The mixture was filtered, and the filtrate was concentrated. The residue was purified by normal phase SiO₂ chromatography (0-8% EtOAc/petroleum ether) to afford 6-(3′-bromo-2,2′-dichloro-[1,1′-biphenyl]-3-yl)-4-chloro-2-methoxynicotinaldehyde (380 mg, 41% yield) as a yellow solid. ¹H NMR (400 MHz, DMSO-d₆): δ 10.35 (s, 1H), 7.88 (d, J₁=2, J₂=7.6, Hz, 1H), 7.78 (d, J₁=1.6, J₂=7.6, Hz, 1H), 7.61 (t, J=7.6 Hz, 2H), 7.55 (s, 1H), 7.51 (d, J₁=1.6, J₂=7.6, Hz, 1H), 7.44 (d, J=2.4, Hz, 1H), 7.42 (d, J=7.6, Hz, 1H), 4.04 (s, 3H).

### (S)-5-((((6-(3′-Bromo-2,2′-dichloro-[1,1′-biphenyl]-3-yl)-4-chloro-2-methoxypyridin-3-yl)methyl)amino)methyl)pyrrolidin-2-one

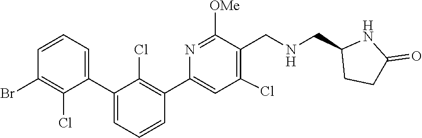 [SMILES: COc1nc(-c2cccc(-c3cccc(Br)c3Cl)c2Cl)cc(Cl)c1CNC[C@@H]1CCC(=O)N1]

To a mixture of 6-(3′-bromo-2,2′-dichloro-[1,1′-biphenyl]-3-yl)-4-chloro-2-methoxynicotinaldehyde (600 mg, 1.27 mmol), (S)-5-(aminomethyl)pyrrolidin-2-one, HCl (383 mg, 2.54 mmol) and trimethyl orthoformate (2 mL) in DCM (30 mL) was added sodium acetate (417 mg, 5.09 mmol). The mixture was stirred at rt for 3 h under N₂. Sodium cyanoborohydride (160 mg, 2.54 mmol) was added and the mixture was stirred at rt for 0.5 h under N₂. The mixture was concentrated to afford (S)-5-((((6-(3′-bromo-2,2′-dichloro-[1,1′-biphenyl]-3-yl)-4-chloro-2-methoxypyridin-3-yl)methyl)amino)methyl)pyrrolidin-2-one (724 mg, crude, m/z: 570 [M+H]⁺) as a white liquid. The crude product was used directly without any purification.

### (S)-tert-Butyl ((6-(3′-bromo-2,2′-dichloro-[1,1′-biphenyl]-3-yl)-4-chloro-2-methoxypyridin-3-yl)methyl)((5-oxopyrrolidin-2-yl)methyl)carbamate

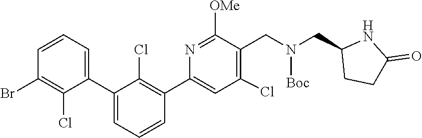 [SMILES: COc1nc(-c2cccc(-c3cccc(Br)c3Cl)c2Cl)cc(Cl)c1CN(C[C@@H]1CCC(=O)N1)C(=O)OC(C)(C)C]

To a mixture of (S)-5-((((6-(3′-bromo-2,2′-dichloro-[1,1′-biphenyl]-3-yl)-4-chloro-2-methoxypyridin-3-yl)methyl)amino)methyl)pyrrolidin-2-one (724 mg, 1.27 mmol) and tert-butoxycarbonyl tert-butyl carbonate (887 mg, 4.07 mmol) in DCM (30 mL) was added triethylamine (0.44 mL, 3.18 mmol). The mixture was stirred at rt for 1 h under N₂. The mixture was filtered, and the filtrate was concentrated to give the residue. The residue was purified by normal phase SiO₂ chromatography (0-8% EtOAc/petroleum ether) to afford (S)-tert-butyl ((6-(3′-bromo-2,2′-dichloro-[1,1′-biphenyl]-3-yl)-4-chloro-2-methoxypyridin-3-yl)methyl)((5-oxopyrrolidin-2-yl)methyl)carbamate (630 mg) as a white solid. ¹H NMR (400 MHz, DMSO-d₆): δ 7.87 (d, J₁=2, J₂=7.6, Hz, 1H), 7.76 (s, 1H), 7.69-7.66 (m, 2H), 7.57 (t, J=7.6 Hz, 1H), 7.46-7.38 (m, 4H), 4.68-4.57 (m, 2H), 3.93 (s, 3H), 3.76 (s, 1H), 3.27 (s, 1H), 2.20-2.02 (m, 4H), 1.67 (s, 1H), 1.43-1.27 (m, 9H).

### (S)-tert-Butyl ((4-chloro-6-(2,2′-dichloro-3′-(4,4,5,5-tetramethyl-1,3,2-dioxaborolan-2-yl)-[1,1′-biphenyl]-3-yl)-2-methoxypyridin-3-yl)methyl)((5-oxopyrrolidin-2-yl)methyl)carbamate

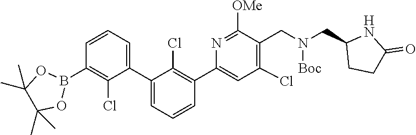 [SMILES: COc1nc(-c2cccc(-c3cccc(B4OC(C)(C)C(C)(C)O4)c3Cl)c2Cl)cc(Cl)c1CN(C[C@@H]1CCC(=O)N1)C(=O)OC(C)(C)C]

To a mixture of (S)-tert-butyl ((6-(3′-bromo-2,2′-dichloro-[1,1′-biphenyl]-3-yl)-4-chloro-2-methoxypyridin-3-yl)methyl)((5-oxopyrrolidin-2-yl)methyl)carbamate (600 mg, 0.89 mmol) and 4,4,4′,4′,5,5,5′,5′-octamethyl-2,2′-bi(1,3,2-dioxaborolane) (455 mg, 1.79 mmol) in dioxane (15 mL) was added potassium acetate (267 mg, 2.69 mmol) and [1,1′-bis(di-tert-butylphosphino) ferrocene]dichloropalladium(II) (73.15 mg, 0.09 mmol). The mixture was stirred at 110° C. for 1 h under N₂. The mixture was filtered, and the filtrate was concentrated. The residue was purified by normal phase SiO₂ chromatography (0-8% EtOAc/petroleum ether) to afford (S)-tert-butyl ((4-chloro-6-(2,2′-dichloro-3′-(4,4,5,5-tetramethyl-1,3,2-dioxaborolan-2-yl)-[1,1′-biphenyl]-3-yl)-2-methoxypyridin-3-yl)methyl)((5-oxopyrrolidin-2-yl)methyl)carbamate (550 mg, 86% yield) as a white solid. ¹H NMR (400 MHz, DMSO-d₆): δ 7.80 (s, 1H), 7.73-7.72 (m, 2H), 7.59 (t, J=8 Hz, 1H), 7.52-7.44 (m, 4H), 4.73-4.63 (m, 2H), 3.98 (s, 3H), 3.82-3.81 (m, 1H), 3.32 (s, 1H), 2.25-2.07 (m, 4H), 1.73 (s, 1H), 1.53-1.32 (m, 21H).

### (R)-tert-Butyl ((4-chloro-6-(2,2′-dichloro-3′-(2-(hydroxymethyl)-3-methyl-4-oxo-3,4-dihydropyrrolo[2,1-f][1,2,4]triazin-6-yl)-[1,1′-biphenyl]-3-yl)-2-methoxypyridin-3-yl)methyl)((5-oxopyrrolidin-2-yl)methyl)carbamate

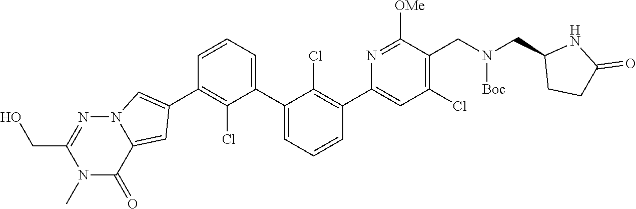 [SMILES: COc1nc(-c2cccc(-c3cccc(-c4cc5c(=O)n(C)c(CO)nn5c4)c3Cl)c2Cl)cc(Cl)c1CN(C[C@@H]1CCC(=O)N1)C(=O)OC(C)(C)C]

To a mixture of 6-bromo-2-(hydroxymethyl)-3-methylpyrrolo[2,1-f][1,2,4]triazin-4(3H)-one (110 mg, 0.43 mmol) and (S)-tert-butyl ((4-chloro-6-(2,2′-dichloro-3′-(4,4,5,5-tetramethyl-1,3,2-dioxaborolan-2-yl)-[1,1′-biphenyl]-3-yl)-2-methoxypyridin-3-yl)methyl)((5-oxopyrrolidin-2-yl)methyl)carbamate (458 mg, 0.64 mmol) in dioxane/water (10:1, 5.5 mL) was added potassium phosphate (271 mg, 1.28 mmol) and ditert-butyl(cyclopentyl)phosphane;dichloropalladium;iron (27.8 mg, 0.04 mmol). The mixture was stirred at 110° C. for 0.5 h under N₂. The mixture was filtered, and the filtrate was concentrated. The residue was purified by normal phase SiO₂ chromatography (0-100% EtOAc/petroleum ether) to afford (R)-tert-butyl ((4-chloro-6-(2,2′-dichloro-3′-(2-(hydroxymethyl)-3-methyl-4-oxo-3,4-dihydropyrrolo[2,1-f][1,2,4]triazin-6-yl)-[1,1′-biphenyl]-3-yl)-2-methoxypyridin-3-yl)methyl)((5-oxopyrrolidin-2-yl)methyl)carbamate (280 mg, 85% yield) as a brown solid. ¹H NMR (400 MHz, DMSO-d₆): δ 8.00 (d, J=1.6 Hz, 1H), 7.76-7.72 (m, 2H), 7.59-7.40 (m, 5H), 7.34 (d, J=7.6 Hz, 1H), 7.26 (d, J=1.6 Hz, 1H), 5.88 (t, J=6 Hz, 1H), 4.65 (s, 2H), 4.52 (d, J=6 Hz, 1H), 3.94 (s, 3H), 3.76 (s, 1H), 3.53 (s, 3H), 3.27 (s, 1H), 2.22-2.02 (m, 4H), 1.68 (s, 1H), 1.43-1.27 (m, 9H).

### (S)-tert-Butyl ((4-chloro-6-(2,2′-dichloro-3′-(2-formyl-3-methyl-4-oxo-3,4-dihydropyrrolo [2,1-f][1,2,4]triazin-6-yl)-[1,1′-biphenyl]-3-yl)-2-methoxypyridin-3-yl)methyl)((5-oxopyrrolidin-2-yl)methyl)carbamate

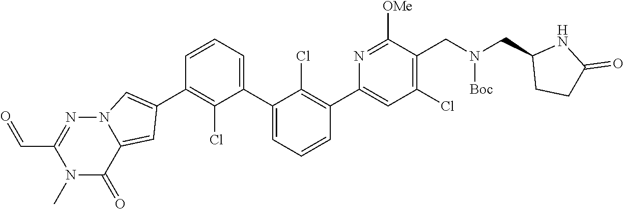 [SMILES: COc1nc(-c2cccc(-c3cccc(-c4cc5c(=O)n(C)c(C=O)nn5c4)c3Cl)c2Cl)cc(Cl)c1CN(C[C@@H]1CCC(=O)N1)C(=O)OC(C)(C)C]

A mixture of (R)-tert-butyl ((4-chloro-6-(2,2′-dichloro-3′-(2-(hydroxymethyl)-3-methyl-4-oxo-3,4-dihydropyrrolo[2,1-f1][1,2,4]triazin-6-yl)-[1,1′-biphenyl]-3-yl)-2-methoxypyridin-3-yl)methyl)((5-oxopyrrolidin-2-yl)methyl)carbamate (260 mg, 0.34 mmol) and Dess-Martin (287 mg, 0.68 mmol) in DCM (3 mL) was stirred at rt for 1 h under N₂. The solvent was evaporated and the residue was purified by normal phase SiO₂ chromatography (0-100% EtOAc/petroleum ether) to afford (S)-tert-butyl ((4-chloro-6-(2,2′-dichloro-3′-(2-formyl-3-methyl-4-oxo-3,4-dihydropyrrolo[2,1-f][1,2,4]triazin-6-yl)-[1,1′-biphenyl]-3-yl)-2-methoxypyridin-3-yl)methyl)((5-oxopyrrolidin-2-yl)methyl)carbamate (170 mg, m/z: 765 [M+H]⁺, observed).

### tert-Butyl ((4-chloro-6-(2,2′-dichloro-3′-(2-((((1r,3r)-3-hydroxy-3-methylcyclobutyl) amino)methyl)-3-methyl-4-oxo-3,4-dihydropyrrolo[2,1-f][1,2,4]triazin-6-yl)-[1,1′-biphenyl]-3-yl)-2-methoxypyridin-3-yl)methyl)(((S)-5-oxopyrrolidin-2-yl)methyl)carbamate

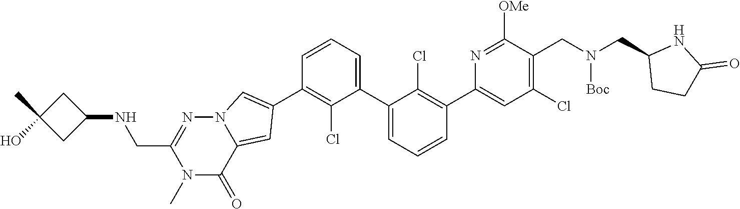

To a mixture of (S)-tert-butyl ((4-chloro-6-(2,2′-dichloro-3′-(2-formyl-3-methyl-4-oxo-3,4-dihydropyrrolo[2,1-f1][1,2,4]triazin-6-yl)-[1,1′-biphenyl]-3-yl)-2-methoxypyridin-3-yl)methyl)((5-oxopyrrolidin-2-yl)methyl)carbamate (100 mg, 0.13 mmol) and (1r,3r)-3-amino-1-methylcyclobutanol, HCl (53.9 mg, 0.39 mmol) DCM/MeOH (1:1, 6 mL) was added sodium acetate (32 mg, 0.39 mmol). The mixture was stirred at rt for 12 h under N₂. Sodium cyanoborohydride (24.6 mg, 0.39 mmol) was added and the mixture was stirred at rt for 0.5 h under N₂. The mixture was concentrated and the residue was purified by prep-TLC (SiO₂, EtOAc:MeOH=6:1) to afford tert-butyl ((4-chloro-6-(2,2′-dichloro-3′-(2-((((1r,3r)-3-hydroxy-3-methylcyclobutyl)amino)methyl)-3-methyl-4-oxo-3,4-dihydropyrrolo[2,1-f][1,2,4]triazin-6-yl)-[1,1′-biphenyl]-3-yl)-2-methoxypyridin-3-yl)methyl)(((S)-5-oxopyrrolidin-2-yl)methyl) carbamate (60 mg, m/z: 850 [M+H]⁺, observed) as a white solid.

### 6-(2,2′-Dichloro-3′-(4-chloro-6-methoxy-5-(((((S)-5-oxopyrrolidin-2-yl)methyl)amino) methyl)pyridin-2-yl)-[1,1′-biphenyl]-3-yl)-2-((((1r,3r)-3-hydroxy-3-methylcyclobutyl) amino)methyl)-3-methylpyrrolo[2,1-f][1,2,4]triazin-4(3H)-one

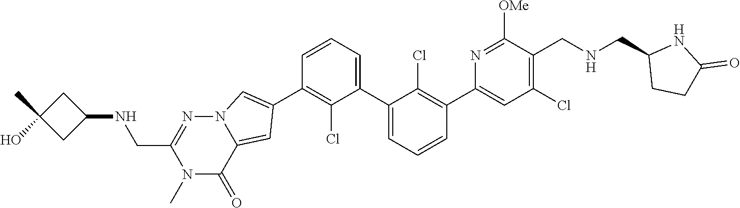

To a mixture of tert-butyl ((4-chloro-6-(2,2′-dichloro-3′-(2-((((1r,3r)-3-hydroxy-3-methylcyclobutyl)amino)methyl)-3-methyl-4-oxo-3,4-dihydropyrrolo[2,1-f][1,2,4]triazin-6-yl)-[1,1′-biphenyl]-3-yl)-2-methoxypyridin-3-yl)methyl)(((S)-5-oxopyrrolidin-2-yl)methyl) carbamate (55 mg, 0.06 mmol) in THE (5 mL) was added TFA (2 mL, 27.01 mmol). The mixture was stirred at 50° C. for 1 h. The mixture was concentrated and the residue was purified by reverse phase HPLC to afford 6-(2,2′-dichloro-3′-(4-chloro-6-methoxy-5-(((((S)-5-oxopyrrolidin-2-yl)methyl)amino)methyl)pyridin-2-yl)-[1,1′-biphenyl]-3-yl)-2-((((1r,3r)-3-hydroxy-3-methylcyclobutyl)amino)methyl)-3-methylpyrrolo[2,1-f][1,2,4]triazin-4(3H)-one (7.4 mg, 15% yield, m/z: 750 [M+H]⁺ observed) as a white solid. ¹H NMR (400 MHz, DMSO-d₆): δ 7.99 (d, J=2 Hz, 1H), 7.74-7.66 (m, 3H), 7.57-7.55 (m, 1H), 7.50-7.46 (m, 2H), 7.40 (s, 1H), 7.34 (dd, J₁=1.6 Hz, J₂=7.6 Hz, 1H), 7.24 (d, J=1.6 Hz, 1H), 4.69 (s, 1H), 3.96 (s, 3H), 3.87 (s, 2H), 3.70 (s, 2H), 3.63-3.62 (m, 1H), 3.55 (s, 3H), 2.57-2.55 (m, 2H), 2.16-2.07 (m, 5H), 1.73-1.66 (m, 3H), 1.25 (s, 3H).

### Example 395: (S)-6-(2,2′-Dichloro-3′-(6-methoxy-5-((((5-oxopyrrolidin-2-yl)methyl)amino)methyl)pyridin-2-yl)-[1,1′-biphenyl]-3-yl)-2-(((2-hydroxyethyl)(methyl)amino)methyl)-3-methylpyrrolo[2,1-f][1,2,4]triazin-4(3H)-one

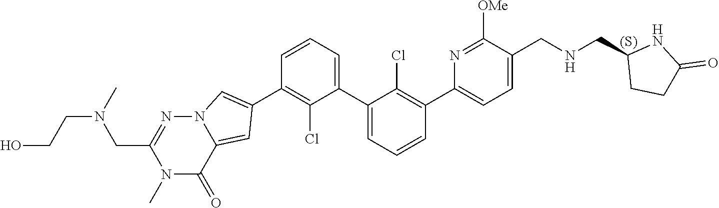

### (S)-tert-Butyl ((6-(2,2′-dichloro-3′-(2-(((2-hydroxyethyl)(methyl)amino)methyl)-3-methyl-4-oxo-3,4-dihydropyrrolo[2,1-f][1,2,4]triazin-6-yl)-[1,1′-biphenyl]-3-yl)-2-methoxypyridin-3-yl)methyl)((5-oxopyrrolidin-2-yl)methyl)carbamate

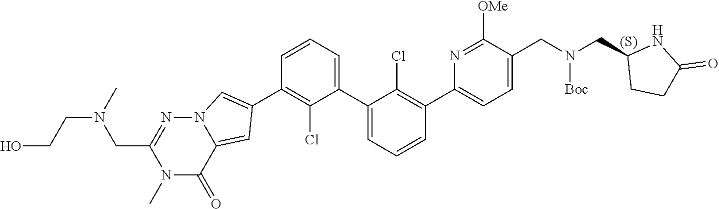

To a mixture (S)-tert-butyl ((6-(2,2′-dichloro-3′-(2-formyl-3-methyl-4-oxo-3,4-dihydropyrrolo [2,1-f][1,2,4]triazin-6-yl)-[1,1′-biphenyl]-3-yl)-2-methoxypyridin-3-yl)methyl)((5-oxopyrrolidin-2-yl)methyl)carbamate (0.2 g, 0.27 mmol) in DCM (5 mL) was added sodium acetate (0.07 g, 0.82 mmol) and 2-(methylamino)ethanol (0.04 g, 0.55 mmol). The mixture was stirred at 25° C. for 11 h. Sodium cyanoborohydride (0.03 g, 0.55 mmol) was added and the mixture was stirred at 25° C. for 1 h. The reaction mixture was concentrated under reduced pressure to give (S)-tert-butyl ((6-(2,2′-dichloro-3′-(2-(((2-hydroxyethyl)(methyl) amino)methyl)-3-methyl-4-oxo-3,4-dihydropyrrolo[2,1-f1][1,2,4]triazin-6-yl)-[1,1′-biphenyl]-3-yl)-2-methoxypyridin-3-yl)methyl)((5-oxopyrrolidin-2-yl)methyl)carbamate as a yellow solid (0.25 g, crude, m/z: 790.1[M+H]⁺ observed).

### (S)-6-(2,2′-Dichloro-3′-(6-methoxy-5-((((5-oxopyrrolidin-2-yl)methyl)amino)methyl)pyridin-2-yl)-[1,1′-biphenyl]-3-yl)-2-(((2-hydroxyethyl)(methyl)amino)methyl)-3-methylpyrrolo[2,1-f][1,2,4]triazin-4(3H)-one

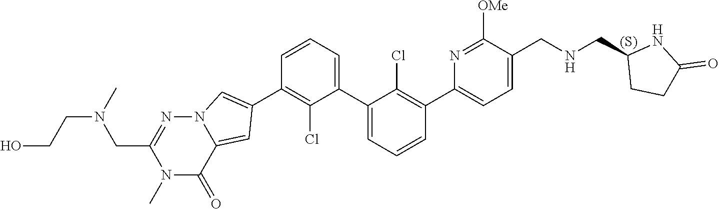

To a mixture (S)-tert-butyl ((6-(2,2′-dichloro-3′-(2-(((2-hydroxyethyl)(methyl)amino)methyl)-3-methyl-4-oxo-3,4-dihydropyrrolo[2,1-f][1,2,4]triazin-6-yl)-[1,1′-biphenyl]-3-yl)-2-methoxy pyridin-3-yl)methyl)((5-oxopyrrolidin-2-yl)methyl)carbamate (0.25 g, 0.32 mmol) in DCM (5 mL) was added trifluoroacetic acid (2.50 mL). The mixture was stirred at 25° C. for 10 min. The reaction mixture was concentrated under reduced pressure and the residue was purified by reverse phase HPLC to afford (S)-6-(2,2′-dichloro-3′-(6-methoxy-5-((((5-oxopyrrolidin-2-yl)methyl)amino)methyl)pyridin-2-yl)-[1,1′-biphenyl]-3-yl)-2-(((2-hydroxyethyl)(methyl) amino)methyl)-3-methylpyrrolo[2,1-f][1,2,4]triazin-4(3H)-one salt as a white solid (10.9 mg, 4% yield, m/z: 690.3[M+H]⁺ observed). ¹H NMR (400 MHz, Methanol-d₄): δ 7.84 (d, J=2.0 Hz, 1H), 7.78 (d, J=7.6 Hz, 1H), 7.65 (dd, J=0.8 Hz, J=7.6 Hz, 2H), 7.52-7.44 (m, 2H), 7.38 (dd, J=1.6 Hz, J=7.6 Hz, 1H), 7.30-7.28 (m, 3H), 4.06 (s, 3H), 3.97-3.88 (m, 3H), 3.72-3.69 (m, 7H), 2.86-2.77 (m, 2H), 2.71 (t, J=5.6 Hz, 2H), 2.40-2.30 (m, 6H), 1.90-1.81 (m, 1H);

### Example 436: 6-(2,2′-dichloro-3′-(6-methoxy-5-(((((S)-5-oxopyrrolidin-2-yl)methyl)amino)methyl)pyridin-2-yl)-[1,1′-biphenyl]-3-yl)-2-((((S)-2-hydroxypropyl)(methyl)amino)methyl)-3-methylpyrrolo[2,1-f][1,2,4]triazin-4(3H)-one

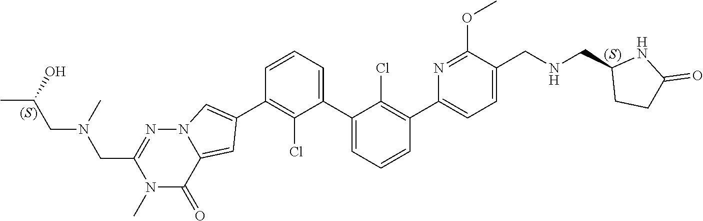

### tert-Butyl ((6-(2,2′-dichloro-3′-(2-((((S)-2-hydroxypropyl)(methyl)amino)methyl)-3-methyl-4-oxo-3,4-dihydropyrrolo[2,1-f][1,2,4]triazin-6-yl)-[1,1′-biphenyl]-3-yl)-2-methoxypyridin-3-yl)methyl)(((S)-5-oxopyrrolidin-2-yl)methyl)carbamate

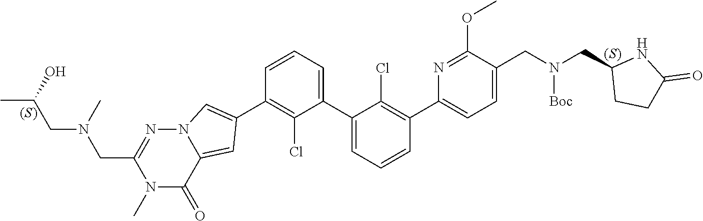

To a mixture of (S)-1-(methylamino)propan-2-ol (0.54 g, 0.61 mmol) in DCM (5 mL) was added (S)-tert-butyl ((6-(2,2′-dichloro-3′-(2-formyl-3-methyl-4-oxo-3,4-dihydropyrrolo[2,1-f][1,2,4]triazin-6-yl)-[1,1′-biphenyl]-3-yl)-2-methoxypyridin-3-yl)methyl)((5-oxopyrrolidin-2-yl)methyl)carbamate (0.15 g, 0.20 mmol), 4A molecular sieves (0.02 g, 0.20 mmol) and glacial acetic acid (0.05 mL). The mixture was degassed and purged with N₂ and stirred at 25° C. for 11.5 h under N₂ atmosphere. Sodium cyanoborohydride (0.02 g, 0.41 mmol) was added and the mixture was stirred at 25° C. for 12 h. Sodium borohydride (0.01 g, 0.41 mmol) was added, and the mixture was stirred at 25° C. for 0.5 h. The reaction mixture was filtered and concentrated under reduced pressure to give tert-butyl ((6-(2,2′-dichloro-3′-(2-((((S)-2-hydroxypropyl) (methyl)amino)methyl)-3-methyl-4-oxo-3,4-dihydropyrrolo[2,1-f]][1,2,4]triazin-6-yl)-[1,1′-biphenyl]-3-yl)-2-methoxypyridin-3-yl)methyl)(((S)-5-oxopyrrolidin-2-yl)methyl)carbamate as a white solid (0.3 g, crude, m/z:804.2[M+H]⁺ observed).

### 6-(2,2′-Dichloro-3′-(6-methoxy-5-(((((S)-5-oxopyrrolidin-2-yl)methyl)amino)methyl)pyridin-2-yl)-[1,1′-biphenyl]-3-yl)-2-((((S)-2-hydroxypropyl)(methyl)amino)methyl)-3-methylpyrrolo[2,1-f][1,2,4]triazin-4(3H)-one

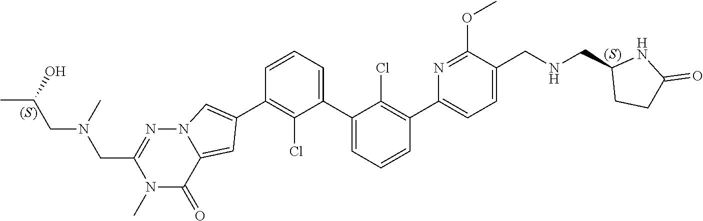

To a mixture of tert-butyl N-[[6-[2-chloro-3-[2-chloro-3-[2-[[[(2S)-2-hydroxypropyl]-methyl-amino]methyl]-3-methyl-4-oxo-pyrrolo[2,1-f][1,2,4]triazin-6-yl]phenyl]phenyl]-2-methoxy-3-pyridyl]methyl]-N-[[(2S)-5-oxopyrrolidin-2-yl]methyl]carbamate (0.3 g, 0.37 mmol) in DCM (3 mL) was added trifluoroacetic acid (1.5 mL). The mixture was stirred at 25° C. for 0.5 h. The reaction mixture was filtered and concentrated under reduced pressure to give a residue. The residue was purified by reverse phase HPLC to afford 6-(2,2′-dichloro-3′-(6-methoxy-5-(((((S)-5-oxopyrrolidin-2-yl)methyl)amino)methyl)pyridin-2-yl)-[1,1′-biphenyl]-3-yl)-2-((((S)-2-hydroxypropyl)(methyl)amino)methyl)-3-methylpyrrolo[2,1-f][1,2,4]triazin-4(3H)-one as a white solid (39.3 mg, m/z:704.3[M+H]⁺ observed). ¹H NMR (400 MHz, Methanol-d₄): δ 7.95 (s, 1H), 7.91 (d, J=7.6 Hz, 1H), 7.68-7.66 (m, 2H), 7.54 (t, J=7.6 Hz, 1H), 7.48 (t, J=7.8 Hz, 1H), 7.43-7.39 (m, 2H), 7.36 (d, J=2.0 Hz, 1H), 7.31 (dd, J=1.4 Hz, J=6.0 Hz, 1H), 4.40-4.33 (m, 3H), 4.13-4.08 (m, 4H), 3.70-3.66 (m, 1H), 3.63-3.50 (m, 4H), 3.29-3.27 (m, 2H), 3.15-3.00 (m, 3H), 2.46-2.37 (m, 3H), 2.17 (s, 2H), 1.95-1.92 (m, 1H), 1.27 (d, J=6.0 Hz, 3H).

### Example 405: (S)-6-(2,2′-Dichloro-3′-(6-methoxy-5-((((5-oxopyrrolidin-2-yl)methyl)amino)methyl)pyridin-2-yl)-[1,1′-biphenyl]-3-yl)-2-(((2-hydroxy-2-methylpropyl)amino)methyl)-3-methylpyrrolo [2,1-f][1,2,4]triazin-4(3H)-one

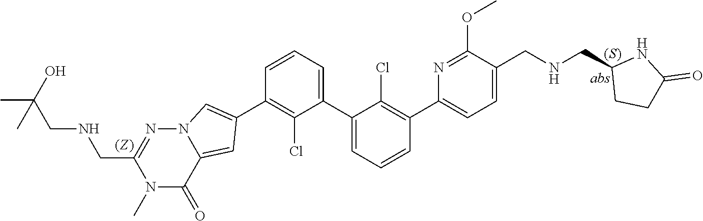

### (S)-tert-Butyl ((6-(2,2′-dichloro-3′-(2-(((2-hydroxy-2-methylpropyl)amino)methyl)-3-methyl-4-oxo-3,4-dihydropyrrolo[2,1-f][1,2,4]triazin-6-yl)-[1,1′-biphenyl]-3-yl)-2-methoxypyridin-3-yl)methyl)((5-oxopyrrolidin-2-yl)methyl)carbamate

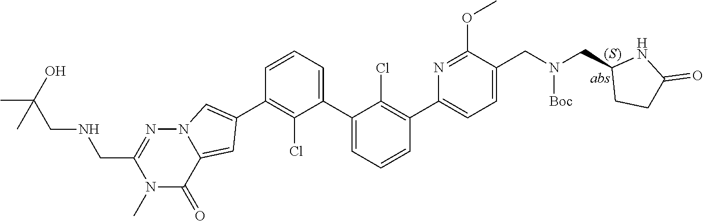

To a mixture of 1-amino-2-methyl-propan-2-ol (0.05 g, 0.61 mmol) in DCM (5 mL) was added (S)-tert-butyl ((6-(2,2′-dichloro-3′-(2-formyl-3-methyl-4-oxo-3,4-dihydropyrrolo[2,1-f]][1,2,4]triazin-6-yl)-[1,1′-biphenyl]-3-yl)-2-methoxypyridin-3-yl)methyl)((5-oxopyrrolidin-2-yl)methyl)carbamate (0.15 g, 0.20 mmol), molecular sieves pack 4A powder (40 mg), and glacial acetic acid (0.05 mL). The mixture was degassed and purged with N₂ and stirred at 25° C. for 12 h under N₂ atmosphere. Sodium cyanoborohydride (0.02 g, 0.41 mmol) was added and the mixture was stirred at 25° C. for 0.5 h under N₂ atmosphere. The reaction mixture was filtered and concentrated under reduced pressure to give a (S)-tert-butyl ((6-(2,2′-dichloro-3′-(2-(((2-hydroxy-2-methylpropyl)amino)methyl)-3-methyl-4-oxo-3,4-dihydropyrrolo[2,1-f1][1,2,4]triazin-6-yl)-[1,1′-biphenyl]-3-yl)-2-methoxypyridin-3-yl)methyl)((5-oxopyrrolidin-2-yl)methyl)carbamate as a yellow solid (0.32 g, crude, m/z: 804.3 [M+H]⁺ observed).

### (S)-6-(2,2′-Dichloro-3′-(6-methoxy-5-((((5-oxopyrrolidin-2-yl)methyl)amino)methyl)pyridin-2-yl)-[1,1′-biphenyl]-3-yl)-2-(((2-hydroxy-2-methylpropyl)amino)methyl)-3-methylpyrrolo[2,1-f][1,2,4]triazin-4(3H)-one

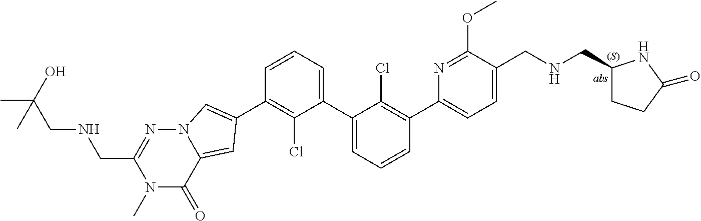

To a mixture of (S)-tert-butyl ((6-(2,2′-dichloro-3′-(2-(((2-hydroxy-2-methylpropyl)amino) methyl)-3-methyl-4-oxo-3,4-dihydropyrrolo[2,1-f][1,2,4]triazin-6-yl)-[1,1′-biphenyl]-3-yl)-2-methoxypyridin-3-yl)methyl)((5-oxopyrrolidin-2-yl)methyl)carbamate (0.32 g, 0.39 mmol) in DCM (3 mL) was added trifluoroacetic acid (1 mL). The mixture was stirred at 25° C. for 0.5 h. The reaction mixture was filtered and concentrated under reduced pressure to give a residue. The residue was purified by reverse phase HPLC to afford (S)-6-(2,2′-dichloro-3′-(6-methoxy-5-((((5-oxopyrrolidin-2-yl)methyl)amino)methyl)pyridin-2-yl)-[1,1′-biphenyl]-3-yl)-2-(((2-hydroxy-2-methylpropyl)amino)methyl)-3-methylpyrrolo[2,1-f][1,2,4]triazin-4(3H)-one as a white solid (35.7 mg, 12% yield, m/z: 704.2 [M+H]⁺ observed). ¹H NMR (400 MHz, Methanol-d₄) δ 7.88 (d, J=2.0 Hz, 1H),7.84 (d, J=7.6 Hz, 1H), 7.66-7.64 (m, 2 H), 7.54-7.45 (m, 2H), 7.40 (dd, J=1.6 Hz, J=6.0 Hz, 1H), 7.35-7.28 (m, 3H), 4.13-4.09 (m, 7H), 3.99-3.97 (m, 1H), 3.62 (s, 3H), 3.03-2.97 (m, 2H), 2.83 (s, 2H), 2.44-2.35 (m, 3H), 1.94-1.86 (m, 1H), 1.29 (s, 6H).

### Example 481: (S)-4-(((6-(2,2′-Dichloro-3′-(6-methoxy-5-(((((S)-5-oxopyrrolidin-2-yl)methyl)amino)methyl) pyridin-2-yl)-[1,1′-biphenyl]-3-yl)-3-methyl-4-oxo-3,4-dihydropyrrolo[2,1-f][1,2,4]triazin-2-yl)methyl)amino)-3-hydroxy-N,N-dimethylbutanamide

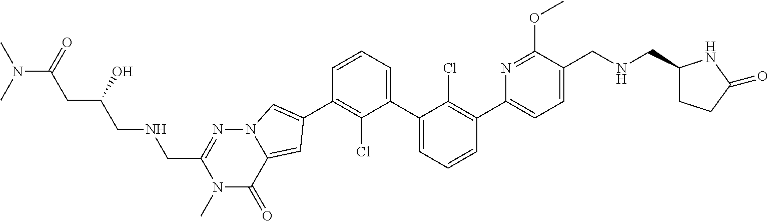 [SMILES: COc1nc(-c2cccc(-c3cccc(-c4cc5c(=O)n(C)c(CNC[C@@H](O)CC(=O)N(C)C)nn5c4)c3Cl)c2Cl)ccc1CNC[C@@H]1CCC(=O)N1]

### (S)-4-((tert-Butoxycarbonyl)amino)-3-hydroxybutanoic acid

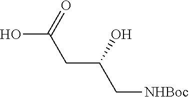 [SMILES: CC(C)(C)OC(=O)NC[C@@H](O)CC(=O)O]

To a solution of (S)-4-amino-3-hydroxybutanoic acid (800 mg, 6.72 mmol) and tert-butoxycarbonyl tert-butyl carbonate (2.20 g, 10.1 mmol) in methanol (10 mL) was added triethylamine (1.40 mL, 10.07 mmol). The mixture was stirred at rt for 12 h. The reaction mixture was concentrated to afford (S)-4-((tert-butoxycarbonyl)amino)-3-hydroxybutanoic acid (2 g, crude) as a white solid. The crude product was used directly without future purification. ¹H NMR (400 MHz, D₂O): δ4.00-3.94 (m, 1H), 3.14-3.05 (m, 1H), 3.03-3.01 (m, 1H), 2.38-2.35 (m, 1H), 2.33-2.27 (m, 1H), 1.35 (s, 9H).

### (S)-tert-Butyl (4-(dimethylamino)-2-hydroxy-4-oxobutyl)carbamate

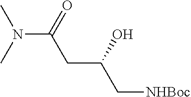 [SMILES: CN(C)C(=O)C[C@H](O)CNC(=O)OC(C)(C)C]

To a mixture of (S)-4-((tert-butoxycarbonyl)amino)-3-hydroxybutanoic acid (500 mg, 2.28 mmol) in DCM (15 mL) was added TEA (1.15 g, 11.40 mmol, 1.59 mL) and [dimethylamino(triazolo[4,5-b]pyridin-3-yloxy)methylidene]-dimethylazanium; hexafluorophosphate (1.04 g, 2.74 mmol). The mixture was stirred at rt for 0.5 h.N-methylmethanamine, HCl (558 mg, 6.84 mmol) was added and the mixture was stirred at rt for 12 h. The reaction mixture was diluted with water (15 mL) and extracted with EtOAc (2×30 ml). The combined organic layers were dried over anhydrous sodium sulfate, filtered and concentrated under reduced pressure. The residue was purified by normal phase SiO₂ chromatography (0-100% EtOAc/petroleum ether) to afford (S)-tert-butyl (4-(dimethylamino)-2-hydroxy-4-oxobutyl)carbamate (350 mg, semi-purified product, m/z: 247 [M+H]⁺, observed) as a yellow oil.

### (S)-4-Amino-3-hydroxy-N,N-dimethylbutanamide

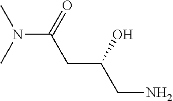

To a mixture of (S)-tert-butyl (4-(dimethylamino)-2-hydroxy-4-oxobutyl)carbamate (500 mg, 2.03 mmol) in DCM (10 mL) was added TFA(5 mL, 67.5 mmol). The mixture was stirred at rt for 0.5 h. The mixture was concentrated to afford (S)-4-amino-3-hydroxy-N,N-dimethylbutanamide (500 mg, crude) as a yellow oil. The crude product was used directly without future purification.

### tert-Butyl ((6-(2,2′-dichloro-3′-(2-((((S)-4-(dimethylamino)-2-hydroxy-4-oxobutyl)amino) methyl)-3-methyl-4-oxo-3,4-dihydropyrrolo[2,1-f][1,2,4]triazin-6-yl)-[1,1′-biphenyl]-3-yl)-2-methoxypyridin-3-yl)methyl)(((S)-5-oxopyrrolidin-2-yl)methyl)carbamate

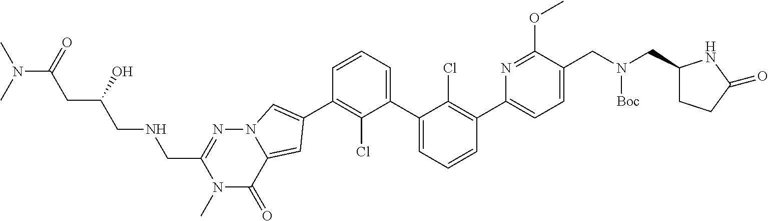 [SMILES: COc1nc(-c2cccc(-c3cccc(-c4cc5c(=O)n(C)c(CNC[C@@H](O)CC(=O)N(C)C)nn5c4)c3Cl)c2Cl)ccc1CN(C[C@@H]1CCC(=O)N1)C(=O)OC(C)(C)C]

To a mixture of(S)-6-(2,2′-dichloro-3′-(6-methoxy-5-((((5-oxopyrrolidin-2-yl)methyl)amino) methyl)pyridin-2-yl)-[1,1′-biphenyl]-3-yl)-3-methyl-4-oxo-3,4-dihydropyrrolo[2,1-f][1,2,4]triazine-2-carbaldehyde (150 mg, 0.21 mmol) and (S)-4-amino-3-hydroxy-N,N-dimethylbutanamide (150 mg, 1.03 mmol) in DCM (3 mL) was added sodium acetate (50.5 mg, 0.62 mmol). The mixture was stirred at rt for 12 h. Sodium cyanoborohydride (39 mg, 0.62 mmol) was added and the mixture was stirred at rt for 0.5 h under N₂. The mixture was concentrated and the residue was purified by prep-TLC (SiO₂, EtOAc:MeOH=6:1) to afford tert-butyl ((6-(2,2′-dichloro-3′-(2-((((S)-4-(dimethylamino)-2-hydroxy-4-oxobutyl)amino) methyl)-3-methyl-4-oxo-3,4-dihydropyrrolo[2,1-f][1,2,4]triazin-6-yl)-[1,1′-biphenyl]-3-yl)-2-methoxypyridin-3-yl)methyl)(((S)-5-oxopyrrolidin-2-yl)methyl)carbamate (40 mg, m/z: 861 [M+H]⁺, observed) as a white solid.

### (S)-4-(((6-(2,2′-Dichloro-3′-(6-methoxy-5-(((((S)-5-oxopyrrolidin-2-yl)methyl)amino) methyl)pyridin-2-yl)-[1,1′-biphenyl]-3-yl)-3-methyl-4-oxo-3,4-dihydropyrrolo[2,1-f][1,2,4]triazin-2-yl)methyl)amino)-3-hydroxy-N,N-dimethylbutanamide

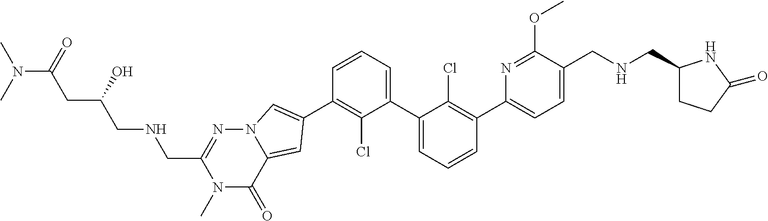 [SMILES: COc1nc(-c2cccc(-c3cccc(-c4cc5c(=O)n(C)c(CNC[C@@H](O)CC(=O)N(C)C)nn5c4)c3Cl)c2Cl)ccc1CNC[C@@H]1CCC(=O)N1]

To a solution of tert-butyl ((6-(2,2′-dichloro-3′-(2-((((S)-4-(dimethylamino)-2-hydroxy-4-oxobutyl)amino)methyl)-3-methyl-4-oxo-3,4-dihydropyrrolo[2,1-f][1,2,4]triazin-6-yl)-[1,1′-biphenyl]-3-yl)-2-methoxypyridin-3-yl)methyl)(((S)-5-oxopyrrolidin-2-yl)methyl)carbamate (35 mg, 0.04 mmol) in DCM (3.5 mL) was added TFA (3.50 mL, 47.3 mmol). The mixture was stirred at rt for 0.5 h under N₂. The mixture was concentrated and the residue was purified by reverse phase HPLC to afford (S)-4-(((6-(2,2′-dichloro-3′-(6-methoxy-5-(((((S)-5-oxopyrrolidin-2-yl)methyl)amino)methyl)pyridin-2-yl)-[1,1′-biphenyl]-3-yl)-3-methyl-4-oxo-3,4-dihydropyrrolo[2,1-f][1,2,4]triazin-2-yl)methyl)amino)-3-hydroxy-N,N-dimethylbutanamide (3 mg, 9% yield, m/z: 761 [M+H]⁺ observed) as a white solid. ¹H NMR (400 MHz, DMSO-d₆): δ 7.98 (d, J=1.2 Hz, 1H), 7.83 (d, J=7.6 Hz, 1H), 7.73-7.66 (m, 3H), 7.54 (t, J=7.6 Hz, 1H), 7.49 (t, J=7.6 Hz, 1H), 7.44-7.42 (m, 1H), 7.34 (d, J=8 Hz, 1H), 7.28 (d, J=7.6 Hz, 1H), 7.23 (d, J=2 Hz, 1H), 4.76 (d, J=4.4 Hz, 1H), 3.96 (m, 1H), 3.93 (s, 3H), 3.82 (s, 2H), 3.73 (s, 2H), 3.65 (s, 1H), 3.55 (s, 3H), 2.95 (s, 3H), 2.79 (s, 3H), 2.43-2.41 (m, 3H), 2.15-2.09 (m, 6H), 1.71-1.69 (m, 1H), 1.24 (s, 1H), 1.15 (s, 2H).

### Example 437: (S)-6-(2,2′-Dichloro-3′-(6-methoxy-5-((((5-oxopyrrolidin-2-yl)methyl)amino)methyl)pyridin-2-yl)-[1,1′-biphenyl]-3-yl)-2-(((3-fluoropropyl)amino)methyl)-3-methylpyrrolo[2,1-f][1,2,4]triazin-4(3H)-one

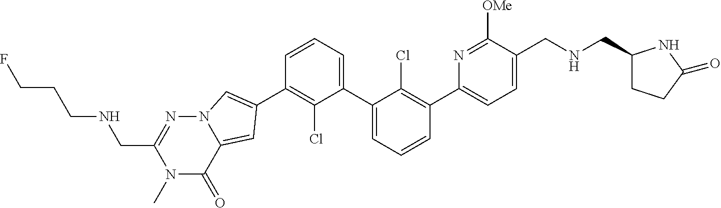 [SMILES: COc1nc(-c2cccc(-c3cccc(-c4cc5c(=O)n(C)c(CNCCCF)nn5c4)c3Cl)c2Cl)ccc1CNC[C@@H]1CCC(=O)N1]

### (S)-tert-Butyl ((6-(2,2′-dichloro-3′-(2-(((3-fluoropropyl)amino)methyl)-3-methyl-4-oxo-3,4-dihydropyrrolo[2,1-f][1,2,4]triazin-6-yl)-[1,1′-biphenyl]-3-yl)-2-methoxypyridin-3-yl)methyl)((5-oxopyrrolidin-2-yl)methyl)carbamate

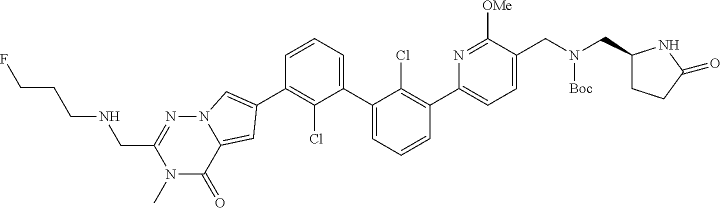 [SMILES: COc1nc(-c2cccc(-c3cccc(-c4cc5c(=O)n(C)c(CNCCCF)nn5c4)c3Cl)c2Cl)ccc1CN(C[C@@H]1CCC(=O)N1)C(=O)OC(C)(C)C]

To a mixture of (S)-tert-butyl ((6-(2,2′-dichloro-3′-(2-formyl-3-methyl-4-oxo-3,4-dihydropyrrolo[2,1-f1][1,2,4]triazin-6-yl)-[1,1′-biphenyl]-3-yl)-2-methoxypyridin-3-yl)methyl) ((5-oxopyrrolidin-2-yl)methyl)carbamate (170 mg, 0.23 mmol) in DCM (0.5 mL) was added 3-fluoropropan-1-amine, HCl (36.9 mg, 0.33 mmol) and sodium acetate (95.3 mg, 1.16 mmol). After 11 h, sodium cyanoborohydride (29.2 mg, 0.46 mmol) was added and stirred for 1 h at rt. The reaction mixture was diluted with water (10 mL) and extracted with DCM (20 ml). The organic layer was concentrated to afford (S)-tert-butyl ((6-(2,2′-dichloro-3′-(2-(((3-fluoropropyl)amino) methyl)-3-methyl-4-oxo-3,4-dihydropyrrolo[2,1-f][1,2,4]triazin-6-yl)-[1,1′-biphenyl]-3-yl)-2-methoxypyridin-3-yl)methyl)((5-oxopyrrolidin-2-yl)methyl)carbamate as a yellow solid (150 mg, crude) which was used without further purification.

### (S)-6-(2,2′-Dichloro-3′-(6-methoxy-5-((((5-oxopyrrolidin-2-yl)methyl)amino)methyl) pyridine-2-yl)-[1,1′-biphenyl]-3-yl)-2-(((3-fluoropropyl)amino)methyl)-3-methylpyrrolo [2,1-f][1,2,4]triazin-4(3H)-one

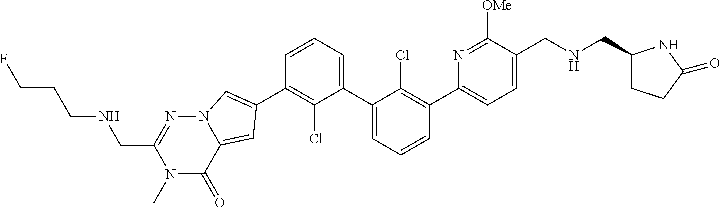 [SMILES: COc1nc(-c2cccc(-c3cccc(-c4cc5c(=O)n(C)c(CNCCCF)nn5c4)c3Cl)c2Cl)ccc1CNC[C@@H]1CCC(=O)N1]

The mixture of (S)-tert-butyl ((6-(2,2′-dichloro-3′-(2-(((3-fluoropropyl)amino)methyl)-3-methyl-4-oxo-3,4-dihydropyrrolo[2,1-f][1,2,4]triazin-6-yl)-[1,1′-biphenyl]-3-yl)-2-methoxypyridin-3-yl)methyl)((5-oxopyrrolidin-2-yl)methyl)carbamate (150 mg, 0.19 mmol) and TFA (1 mL, 13.5 mmol) in DCM (1 mL) was stirred for 0.5 h at rt. The mixture was concentrated in vacuo and the residue was purified by reverse phase HPLC to afford (S)-6-(2,2′-dichloro-3′-(6-methoxy-5-((((5-oxopyrrolidin-2-yl)methyl)amino)methyl)pyridin-2-yl)-[1,1′-biphenyl]-3-yl)-2-(((3-fluoropropyl)amino)methyl)-3-methylpyrrolo[2,1-f][1,2,4]triazin-4(3H)-one as a white solid (18 mg, 12% yield). m/z: 691 [M]⁺, 693 [M+2]⁺ observed. ¹H NMR (400 MHz, DMSO-d₆): δ 8.14 (s, 1H), 7.98 (d, J=1.6 Hz, 1H), 7.84 (d, J=7.2 Hz, 1 H), 7.73-7.66 (m, 3H), 7.57-7.47 (m, 2H), 7.44-7.42 (m, 1H), 7.35-7.33 (m, 1H), 7.29 (d, J=7.6 Hz, 1H), 7.24 (d, J=1.6 Hz, 1H), 4.57 (t, J=6.4 Hz, 1H), 4.45 (d, J=6.0 Hz, 1H), 3.93 (s, 3H), 3.80 (s, 2H), 3.77 (s, 2H), 3.54 (s, 3H), 2.71 (t, J=6.8 Hz, 3H), 2.61 (d, J=6.0 Hz, 2H), 2.16-2.09 (m, 3H), 1.87-1.77 (m, 2H), 1.72-1.70 (m, 1H).

### Example 465: (S)-6-(2,2′-Dichloro-3′-(6-methoxy-5-((((5-oxopyrrolidin-2-yl)methyl)amino)methyl)pyridin-2-yl)-[1,1′-biphenyl]-3-yl)-2-(((3,3-difluoropropyl)amino)methyl)-3-methylpyrrolo[2,1-f][1,2,4]triazin-4(3H)-one

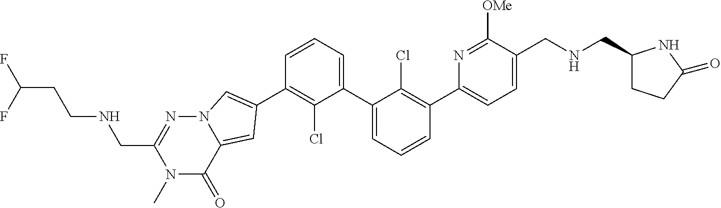 [SMILES: COc1nc(-c2cccc(-c3cccc(-c4cc5c(=O)n(C)c(CNCCC(F)F)nn5c4)c3Cl)c2Cl)ccc1CNC[C@@H]1CCC(=O)N1]

### (S)-tert-Butyl ((6-(2,2′-dichloro-3′-(2-(((3,3-difluoropropyl)amino)methyl)-3-methyl-4-oxo-3,4-dihydropyrrolo[2,1-f][1,2,4]triazin-6-yl)-[1,1′-biphenyl]-3-yl)-2-methoxypyridin-3-yl)methyl)((5-oxopyrrolidin-2-yl)methyl)carbamate

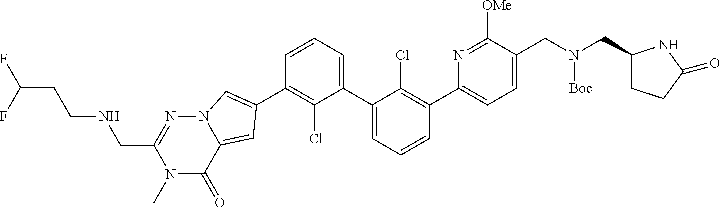 [SMILES: COc1nc(-c2cccc(-c3cccc(-c4cc5c(=O)n(C)c(CNCCC(F)F)nn5c4)c3Cl)c2Cl)ccc1CN(C[C@@H]1CCC(=O)N1)C(=O)OC(C)(C)C]

To a mixture of (S)-tert-butyl ((6-(2,2′-dichloro-3′-(2-formyl-3-methyl-4-oxo-3,4-dihydropyrrolo [2,1-f][1,2,4]triazin-6-yl)-[1,1′-biphenyl]-3-yl)-2-methoxypyridin-3-yl)methyl)((5-oxopyrrolidin-2-yl)methyl)carbamate (0.14 g, 0.19 mmol) and 3,3-difluoropropan-1-amine, HCl (0.05 g, 0.38 mmol) in DCM (3 mL) was added sodium acetate (0.05 g, 0.57 mmol) in one portion at rt under N₂. The mixture was stirred at rt for 0.5 h. Sodium borohydride (0.04 g, 0.57 mmol) was added and the mixture was stirred at rt for 0.5 h. The mixture was concentrated to afford (S)-tert-butyl ((6-(2,2′-dichloro-3′-(2-(((3,3-difluoropropyl)amino) methyl)-3-methyl-4-oxo-3,4-dihydropyrrol o[2,1-f][1,2,4]triazin-6-yl)-[1,1′-biphenyl]-3-yl)-2-methoxypyridin-3-yl)methyl)((5-oxopyrrolidin-2-yl)methyl)carbamate as a crude yellow gum (0.3 g, crude, m/z: 810 [M+H]⁺).

### (S)-6-(2,2′-Dichloro-3′-(6-methoxy-5-((((5-oxopyrrolidin-2-yl)methyl)amino)methyl)pyridin-2-yl)-[1,1′-biphenyl]-3-yl)-2-(((3,3-difluoropropyl)amino)methyl)-3-methylpyrrolo[2,1-f][1,2,4]triazin-4(3H)-one

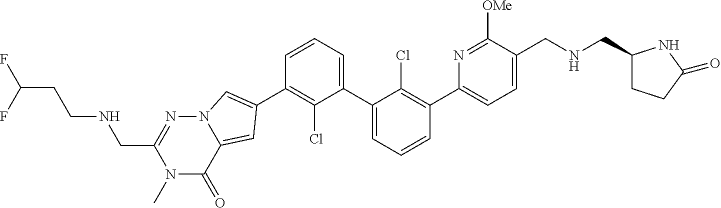 [SMILES: COc1nc(-c2cccc(-c3cccc(-c4cc5c(=O)n(C)c(CNCCC(F)F)nn5c4)c3Cl)c2Cl)ccc1CNC[C@@H]1CCC(=O)N1]

To a mixture of (S)-tert-butyl ((6-(2,2′-dichloro-3′-(2-(((3,3-difluoropropyl)amino)methyl)-3-methyl-4-oxo-3,4-dihydropyrrolo[2,1-f][1,2,4]triazin-6-yl)-[1,1′-biphenyl]-3-yl)-2-methoxypyridin-3-yl)methyl)((5-oxopyrrolidin-2-yl)methyl)carbamate (0.3 g, 0.3 mmol) in DCM (0.6 mL) was added trifluoroacetic acid (1.5 mL, 20.26 mmol). The mixture was stirred at rt for 0.5 h. The mixture was concentrated and the residue was purified by reverse phase HPLC to afford (S)-6-(2,2′-dichloro-3′-(6-methoxy-5-((((5-oxopyrrolidin-2-yl)methyl)amino) methyl)pyridin-2-yl)-[1,1′-biphenyl]-3-yl)-2-(((3,3-difluoropropyl)amino)methyl)-3-methylpyrrolo[2,1-f][1,2,4]triazin-4(3H)-one as a white solid (10.5 mg, 3% yield, m/z: 710 [M+H]⁺ observed). ¹H NMR (400 MHz, DMSO-d₆): δ 7.99 (d, J=2 Hz, 1H), 7.87-7.85 (m, 1H), 7.74-7.67 (m, 3H), 7.58-7.44 (m, 3H), 7.36-7.30 (m, 2H), 7.25 (d, J=2 Hz, 1H), 6.30-6.00 (m, 1H), 3.95 (s, 3H), 3.81-3.70 (m, 4H), 3.54 (s, 3H), 2.75 (t, J=6 Hz, 2H), 2.76-2.65 (m, 3H), 2.17-1.97 (m, 5H), 1.74-1.72 (m, 1H).

### Example 406: (S)-6-(2,2′-Dichloro-3′-(6-methoxy-5-((((5-oxopyrrolidin-2-yl)methyl)amino)methyl)pyridin-2-yl)-[1,1′-biphenyl]-3-yl)-3-methyl-2-(((3,3,3-trifluoropropyl)amino)methyl)pyrrolo[2,1-f]1[1,2,4]triazin-4(3H)-one

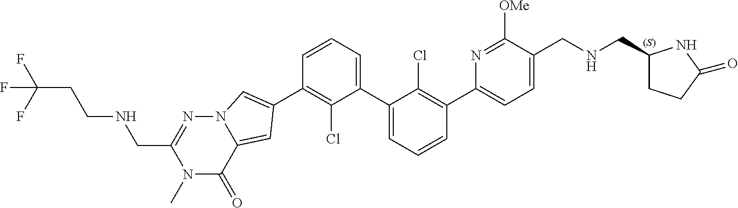

### (S)-tert-Butyl ((6-(2,2′-dichloro-3′-(3-methyl-4-oxo-2-(((3,3,3-trifluoropropyl)amino)methyl)-3,4-dihydropyrrolo[2,1-f][1,2,4]triazin-6-yl)-[1,1′-biphenyl]-3-yl)-2-methoxypyridin-3-yl)methyl)((5-oxopyrrolidin-2-yl)methyl)carbamate

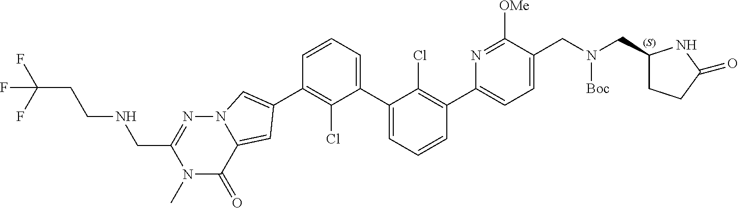

To a mixture (S)-tert-butyl ((6-(2,2′-dichloro-3′-(2-formyl-3-methyl-4-oxo-3,4-dihydropyrrolo [2,1-f][1,2,4]triazin-6-yl)-[1,1′-biphenyl]-3-yl)-2-methoxypyridin-3-yl)methyl)((5-oxopyrrolidin-2-yl)methyl)carbamate (0.15 g,0.2 mmol) in DCM (4 mL) was added sodium acetate (0.04 g, 0.51 mmol) and 3,3,3-trifluoropropan-1-amine, HCl (0.06 g, 0.41 mmol). The mixture was stirred at 25° C. for 11 h. Sodium cyanoborohydride (0.03 g, 0.41 mmol) was added and the mixture was stirred at 25° C. for 1 h. The reaction mixture was concentrated under reduced pressure to give (S)-tert-butyl ((6-(2,2′-dichloro-3′-(3-methyl-4-oxo-2-(((3,3,3-trifluoropropyl) amino)methyl)-3,4-dihydropyrrolo[2,1-f][1,2,4]triazin-6-yl)-[1,1′-biphenyl]-3-yl)-2-methoxypyridin-3-yl)methyl)((5-oxopyrrolidin-2-yl)methyl)carbamate as a yellow solid (0.2 g, crude, m/z: 828.3.1[M+H]⁺ observed).

### (S)-6-(2,2′-Dichloro-3′-(6-methoxy-5-((((5-oxopyrrolidin-2-yl)methyl)amino)methyl)pyridin-2-yl)-[1,1′-biphenyl]-3-yl)-3-methyl-2-(((3,3,3-trifluoropropyl)amino)methyl)pyrrolo[2,1-f][1,2,4]triazin-4(3H)-one

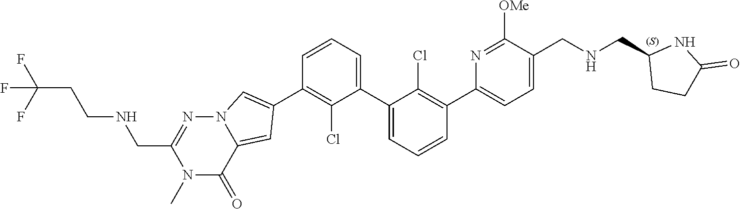

To a solution of (S)-tert-butyl ((6-(2,2′-dichloro-3′-(3-methyl-4-oxo-2-(((3,3,3-trifluoropropyl) amino)methyl)-3,4-dihydropyrrolo[2,1-f][1,2,4]triazin-6-yl)-[1,1′-biphenyl]-3-yl)-2-methoxypyridin-3-yl)methyl)((5-oxopyrrolidin-2-yl)methyl)carbamate (0.2 g, 0.24 mmol) in DCM (2 mL) was added trifluoroacetic acid (1 mL). The mixture was stirred at 25° C. for 10 min. The reaction mixture was concentrated and the residue was purified by reverse phase HPLC to afford (S)-6-(2,2′-dichloro-3′-(6-methoxy-5-((((5-oxopyrrolidin-2-yl)methyl)amino)methyl) pyridin-2-yl)-[1,1′-biphenyl]-3-yl)-3-methyl-2-(((3,3,3-trifluoropropyl)amino)methyl)pyrrolo [2,1-f][1,2,4]triazin-4(3H)-one as a yellow solid (34.3 mg, 18% yield, m/z: 728.1[M+H]⁺ observed)¹H NMR (400 MHz, Methanol-d₄) δ 7.86 (d, J=1.6 Hz, 1H), 7.82 (d, J=7.6 Hz, 1H), 7.65 (dd, J=1.6 Hz, J=8 Hz, 2H), 7.53-7.44 (m, 2H), 7.39 (dd, J=1.6 Hz, J=6 Hz, 1H), 7.34-7.28 (m, 3H), 4.14-4.05 (m, 5H), 3.95 (s, 3H), 3.60 (s, 3H), 3.04-2.94 (m, 4H), 2.51-217 (m, 5H), 1.93-1.85 (m, 1H)

### Example 478: (S)-6-(2,2′-Dichloro-3′-(4-chloro-6-methoxy-5-((((5-oxopyrrolidin-2-yl)methyl)amino) methyl)pyridin-2-yl)-[1,1′-biphenyl]-3-yl)-2-(((2-hydroxy-2-methylpropyl)amino)methyl)-3-methylpyrrolo[2,1-f][1,2,4]triazin-4(3H)-one

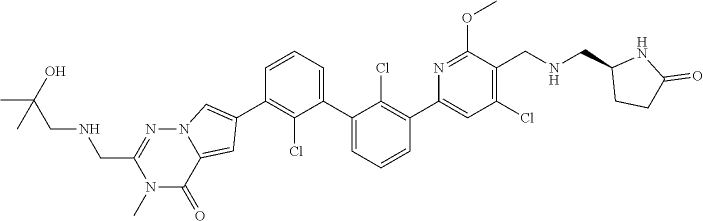 [SMILES: COc1nc(-c2cccc(-c3cccc(-c4cc5c(=O)n(C)c(CNCC(C)(C)O)nn5c4)c3Cl)c2Cl)cc(Cl)c1CNC[C@@H]1CCC(=O)N1]

### (S)-tert-Butyl ((4-chloro-6-(2,2′-dichloro-3′-(2-(((2-hydroxy-2-methylpropyl)amino)methyl)-3-methyl-4-oxo-3,4-dihydropyrrolo12,1-f][1,2,4]triazin-6-yl)-)1,1′-biphenyl]-3-yl)-2-methoxypyridin-3-yl)methyl)((5-oxopyrrolidin-2-yl)methyl)carbamate

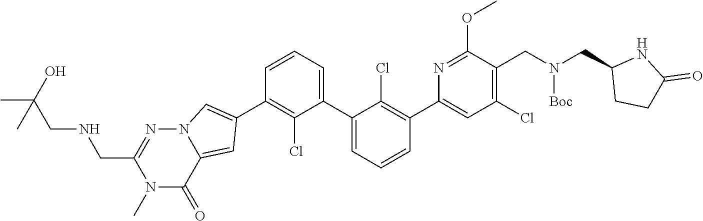 [SMILES: COc1nc(-c2cccc(-c3cccc(-c4cc5c(=O)n(C)c(CNCC(C)(C)O)nn5c4)c3Cl)c2Cl)cc(Cl)c1CN(C[C@@H]1CCC(=O)N1)C(=O)OC(C)(C)C]

To a mixture of (S)-tert-butyl ((4-chloro-6-(2,2′-dichloro-3′-(2-formyl-3-methyl-4-oxo-3,4-dihydropyrrolo[2,1-1′][1,2,4]triazin-6-yl)-[1,1′-biphenyl]-3-yl)-2-methoxypyridin-3-yl)methyl)((5-oxopyrrolidin-2-yl)methyl)carbamate (40 mg, 52.2 umol) and 1-amino-2-methyl-propan-2-ol (14 mg, 157 umol) in DCM/MeOH mixture (1:1, 4 mL) was added sodium acetate (13 mg, 157 umol). The mixture was stirred at rt for 12 h. Sodium cyanoborohydride (10 mg, 157 umol) was added and the mixture was stirred at rt for 0.5 h under N₂. The mixture was concentrated to afford (S)-tert-butyl ((4-chloro-6-(2,2′-dichloro-3′-(2-(((2-hydroxy-2-methylpropyl)amino)methyl)-3-methyl-4-oxo-3,4-dihydropyrrolo[2,1-f1][1,2,4]triazin-6-yl)-[1,1′-biphenyl]-3-yl)-2-methoxypyridin-3-yl)methyl)((5-oxopyrrolidin-2-yl)methyl)carbamate (40 mg, crude, m/z: 838 [M+H]⁺, observed) as a yellow oil. The crude product was used for next step directly.

### (S)-6-(2,2′-Dichloro-3′-(4-chloro-6-methoxy-5-((((5-oxopyrrolidin-2-yl)methyl)amino) methyl)pyridin-2-yl)-[1,1′-biphenyl]-3-yl)-2-(((2-hydroxy-2-methylpropyl)amino)methyl)-3-methylpyrrolo[2,1-f][1,2,4]triazin-4(3H)-one

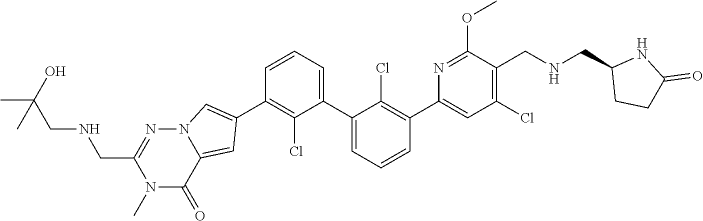 [SMILES: COc1nc(-c2cccc(-c3cccc(-c4cc5c(=O)n(C)c(CNCC(C)(C)O)nn5c4)c3Cl)c2Cl)cc(Cl)c1CNC[C@@H]1CCC(=O)N1]

To a solution of (S)-tert-butyl ((4-chloro-6-(2,2′-dichloro-3′-(2-(((2-hydroxy-2-methylpropyl) amino)methyl)-3-methyl-4-oxo-3,4-dihydropyrrolo[2,1-f][1,2,4]triazin-6-yl)-[1,1′-biphenyl]-3-yl)-2-methoxypyridin-3-yl)methyl)((5-oxopyrrolidin-2-yl)methyl)carbamate (40 mg, 47.7 umol) in DCM (1 mL) was added TFA (2 mL, 27 mmol). The mixture was stirred at 50° C. for 1 h. The mixture was concentrated and the residue was purified by reverse phase HPLC to afford (S)-6-(2,2′-dichloro-3′-(4-chloro-6-methoxy-5-((((5-oxopyrrolidin-2-yl)methyl)amino)methyl)pyridin-2-yl)-[1,1′-biphenyl]-3-yl)-2-(((2-hydroxy-2-methylpropyl)amino)methyl)-3-methylpyrrolo[2,1-f][1,2,4]triazin-4(3H)-one (3.6 mg, 9% yield, m/z: 738 [M+H]⁺ observed) as a white solid. ¹H NMR (400 MHz, Methanol-d₄): δ 7.95 (s, 1H), 7.70-7.66 (m, 2H), 7.55 (t, J=7.6 Hz, 1H), 7.51-7.43 (m, 3H), 7.37 (t, J=1.6 Hz, 1H), 7.31 (dd, J₁=1.6 Hz, J₁=7.6 Hz, 1H), 4.50-4.45 (m, 3H), 4.15 (s, 3H), 4.08-4.06 (m, 1H), 3.56 (s, 3H), 3.27-3.15 (m, 4H), 3.46-3.38 (m, 3H), 1.94-1.92 (m, 1H), 1.40 (s, 6H).

### Example 466: (S)-6-(2,2′-Dichloro-3′-(6-methoxy-5-((((5-oxopyrrolidin-2-yl)methyl)amino)methyl)pyridin-2-yl)-[1,1′-biphenyl]-3-yl)-3-methyl-2-(((3,3,3-trifluoro-2,2-dimethylpropyl)amino)methyl) pyrrolo[2,1-f][1,2,4]triazin-4(3H)-one

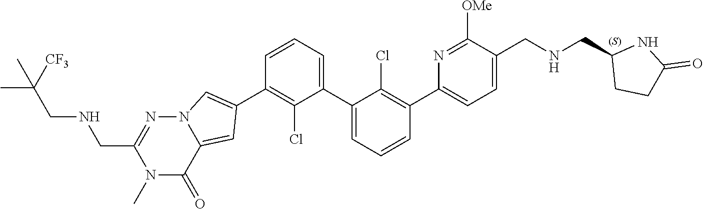

### (S)-tert-Butyl ((6-(2,2′-dichloro-3′-(3-methyl-4-oxo-2-(((3,3,3-trifluoro-2,2-dimethylpropyl) amino)methyl)-3,4-dihydropyrrolo[2,1-f][1,2,4]triazin-6-yl)-[1,1′-biphenyl]-3-yl)-2-methoxypyridin-3-yl)methyl)((5-oxopyrrolidin-2-yl)methyl)carbamate

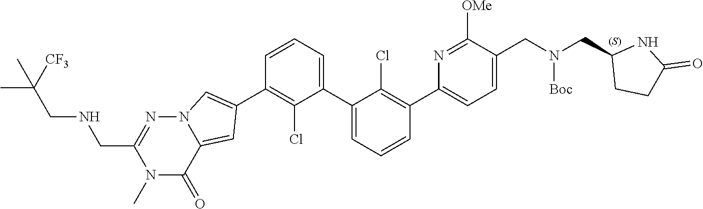

To a mixture of (S)-tert-butyl ((6-(2,2′-dichloro-3′-(2-formyl-3-methyl-4-oxo-3,4-dihydropyrrolo [2,1-f][1,2,4]triazin-6-yl)-[1,1′-biphenyl]-3-yl)-2-methoxypyridin-3-yl)methyl)((5-oxopyrrolidin-2-yl)methyl)carbamate (0.14 g, 0.19 mmol) and 3,3,3-trifluoro-2,2-dimethyl-propan-1-amine, HCl (0.07 g, 0.38 mmol) in CH₂Cl₂ (3 mL) was added sodium acetate (0.05 g, 0.57 mmol). The mixture was stirred at rt for 40 min. Sodium cyanoborohydride (0.04 g, 0.57 mmol) was added and the mixture was stirred at rt for 20 min. The mixture was concentrated to afford (S)-tert-butyl ((6-(2,2′-dichloro-3′-(3-methyl-4-oxo-2-(((3,3,3-trifluoro-2,2-dimethylpropyl)amino) methyl)-3,4-dihydropyrrolo[2,1-f][1,2,4]triazin-6-yl)-[1,1′-biphenyl]-3-yl)-2-methoxypyridin-3-yl)methyl)((5-oxopyrrolidin-2-yl)methyl)carbamate as a yellow gum (280 mg, crude, m/z: 856 [M+H]⁺ observed).

### (S)-6-(2,2′-Dichloro-3′-(6-methoxy-5-((((5-oxopyrrolidin-2-yl)methyl)amino)methyl)pyridin-2-yl)-[1,1′-biphenyl]-3-yl)-3-methyl-2-(((3,3,3-trifluoro-2,2-dimethylpropyl)amino) methyl)pyrrolo[2,1-f][1,2,4]triazin-4(3H)-one

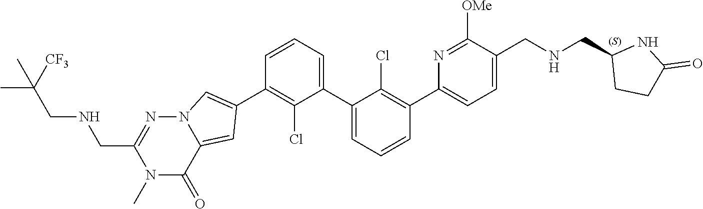

To a mixture of (S)-tert-butyl ((6-(2,2′-dichloro-3′-(3-methyl-4-oxo-2-(((3,3,3-trifluoro-2,2-dimethylpropyl)amino)methyl)-3,4-dihydropyrrolo[2,1-f][1,2,4]triazin-6-yl)-[1,1′-biphenyl]-3-yl)-2-methoxypyridin-3-yl)methyl)((5-oxopyrrolidin-2-yl)methyl)carbamate (0.28 g, 0.33 mmol) in CH₂Cl₂ (1 mL) was added trifluoroacetic acid (2 mL, 27 mmol). The mixture was stirred at rt for 0.5 h. The mixture was concentrated and the residue was purified by reverse phase HPLC to afford (S)-6-(2,2′-dichloro-3′-(6-methoxy-5-((((5-oxopyrrolidin-2-yl)methyl) amino)methyl)pyridin-2-yl)-[1,1′-biphenyl]-3-yl)-3-methyl-2-(((3,3,3-trifluoro-2,2-dimethyl propyl)amino)methyl)pyrrolo[2,1-f][1,2,4]triazin-4(3H)-one as a white solid (64.2 mg, 24% yield, m/z: 756 [M+H]⁺ observed). ¹H NMR (400 MHz, DMSO-d₆): δ 8.00-7.97 (m, 1H), 7.90 (d, J=7.2 Hz, 1H), 7.73 (d, J=8 Hz, d, J=1.6 Hz, 1H), 7.67 (d, J=7.6 Hz, d, J=1.6 Hz, 1H), 7.63 (bs, 1H), 7.57 (t, J=7.6 Hz, 1H), 7.52-7.44 (m, 2H), 7.36-7.33 (m, 2H), 7.24 (d, J=2 Hz, 1H), 4.03-3.79 (m, 8H), 3.55-3.52 (m, 3H), 2.88 (br s, 2H), 2.71 (s, 2H), 2.22-2.09 (m, 3H), 1.77-1.75 (m, 1H), 1.08 (s, 6H).

### Example 462: (S)-6-(2,2′-Dichloro-3′-(6-methoxy-5-((((5-oxopyrrolidin-2-yl)methyl)amino)methyl)pyridin-2-yl)-[1,1′-biphenyl]-3-yl)-2-((3-hydroxy-3-methylazetidin-1-yl)methyl)-3-methylpyrrolo [2,1-f][1,2,4]triazin-4(3H)-one

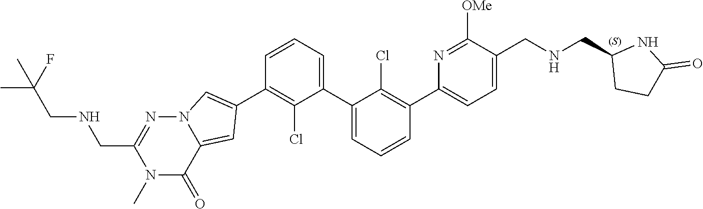

### (S)-tert-Butyl ((6-(2,2′-dichloro-3′-(2-(((2-fluoro-2-methylpropyl)amino)methyl)-3-methyl-4-oxo-3,4-dihydropyrrolo[2,1-f][1,2,4]triazin-6-yl)-[1,1′-biphenyl]-3-yl)-2-methoxypyridin-3-yl)methyl)((5-oxopyrrolidin-2-yl)methyl)carbamate

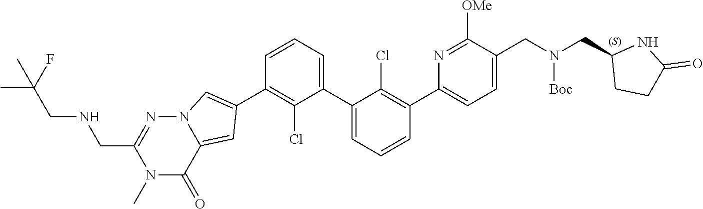

To a solution of (S)-tert-butyl ((6-(2,2′-dichloro-3′-(2-formyl-3-methyl-4-oxo-3,4-dihydropyrrolo[2,1-f][1,2,4]triazin-6-yl)-[1,1′-biphenyl]-3-yl)-2-methoxypyridin-3-yl)methyl) ((5-oxopyrrolidin-2-yl)methyl)carbamate (0.15 g, 0.2 mmol) in DCM (30 mL) was added 4A MS (0.15 g), 2-fluoro-2-methyl-propan-1-amine, HCl (0.05 g, 0.4 mmol) and sodium acetate (0.03 g, 0.4 mmol). The mixture was stirred at 25° C. for 11 h. Sodium cyanoborohydride (0.03 g, 0.4 mmol) was added and the mixture was stirred at 25° C. for 1 h. The reaction mixture was concentrated under reduced pressure to give (S)-tert-butyl ((6-(2,2′-dichloro-3′-(2-(((2-fluoro-2-methylpropyl)amino)methyl)-3-methyl-4-oxo-3,4-dihydropyrrolo[2,1-f][1,2,4]triazin-6-yl)-[1,1′-biphenyl]-3-yl)-2-methoxypyridin-3-yl)methyl) ((5-oxopyrrolidin-2-yl)methyl)carbamate as a white solid (0.2 g, crude, m/z: 806.3 [M+H]⁺ observed).

### (S)-6-(2,2′-Dichloro-3′-(6-methoxy-5-((((5-oxopyrrolidin-2-yl)methyl)amino)methyl)pyridin-2-yl)-[1,1′-biphenyl]-3-yl)-2-(((2-fluoro-2-methylpropyl)amino)methyl)-3-methylpyrrolo[2,1-f][1,2,4]triazin-4(3H)-one

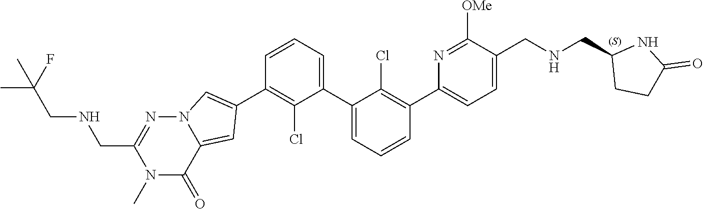

To a solution of (S)-tert-butyl ((6-(2,2′-dichloro-3′-(2-(((2-fluoro-2-methylpropyl) amino)methyl)-3-methyl-4-oxo-3,4-dihydropyrrolo[2,1-f][1,2,4]triazin-6-yl)-[1,1′-biphenyl]-3-yl)-2-methoxypyridin-3-yl)methyl)((5-oxopyrrolidin-2-yl)methyl)carbamate (0.2 g, 0.17 mmol) in DCM (2 mL) was added TFA (1.2 mL). The mixture was stirred at 25° C. for 0.5 h.

The reaction mixture was concentrated and the residue was purified by reverse phase HPLC to afford (S)-6-(2,2′-dichloro-3′-(6-methoxy-5-((((5-oxopyrrolidin-2-yl)methyl)amino)methyl)pyridin-2-yl)-[1,1′-biphenyl]-3-yl)-2-(((2-fluoro-2-methylpropyl)amino)methyl)-3-methylpyrrolo[2,1-f][1,2,4]triazin-4(3H)-one as a white solid (33 mg, 25% yield, m/z: 706.3[M+H]⁺ observed). ¹H NMR (400 MHz, Methanol-d₄) δ 7.83-7.86 (m, 2H), 7.66 (dd, J=1.6 Hz, J=8 Hz, 2H), 7.44-7.54 (m, 2H), 7.40 (dd, J=1.6 Hz, J=7.6 Hz, 1H), 7.35 (d, J=7.2 Hz, 1H), 7.28-7.30 (m, 2H), 3.98-4.17 (m, 8H), 3.65 (s, 3H), 2.87-3.15 (m, 4H), 2.36-2.46 (m, 3H), 1.90-1.91 (m, 1H), 1.46 (s, 3H), 1.37 (s, 3H).

### Example 463: (S)-6-(2,2′-Dichloro-3′-(6-methoxy-5-((((5-oxopyrrolidin-2-yl)methyl)amino)methyl)pyridin-2-yl)-[1,1′-biphenyl]-3-yl)-2-((((1-fluorocyclopropyl)methyl)amino)methyl)-3-methylpyrrolo[2,1-f][1,2,4]triazin-4(3H)-one

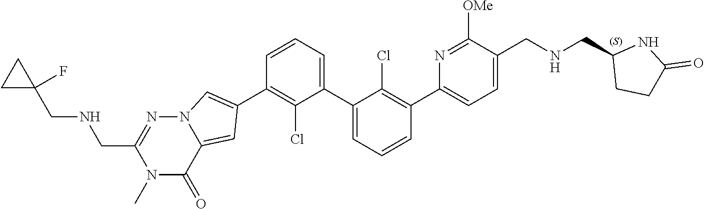

### (S)-tert-Butyl ((6-(2,2′-dichloro-3′-(2-((((1-fluorocyclopropyl)methyl)amino)methyl)-3-methyl-4-oxo-3,4-dihydropyrrolo[2,1-f][1,2,4]triazin-6-yl)-[1,1′-biphenyl]-3-yl)-2-methoxypyridin-3-yl)methyl)((5-oxopyrrolidin-2-yl)methyl)carbamate

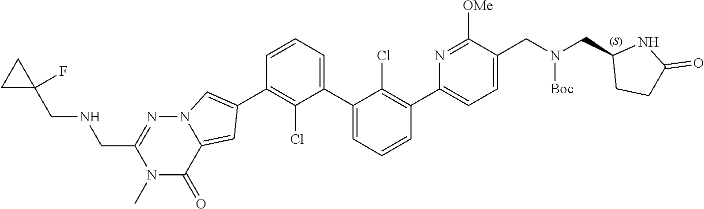

To a solution of tert-butyl (S)-tert-butyl ((4-chloro-6-(2,2′-dichloro-3′-(2-formyl-3-methyl-4-oxo-3,4-dihydropyrrolo[2,1-f][1,2,4]triazin-6-yl)-[1,1′-biphenyl]-3-yl)-2-methoxypyridin-3-yl)methyl)((5-oxopyrrolidin-2-yl)methyl)carbamate (0.15 g, 0.2 mmol) in DCM (30 mL) was added 4A MS (0.15 g), (1-fluorocyclopropyl)methanamine, HCl (0.05 g, 0.4 mmol) and sodium acetate (0.03 g, 0.4 mmol). The mixture was stirred at 25° C. for 11 h. Sodium cyanoborohydride (0.03 g, 0.4 mmol) was added and the resulting mixture was stirred at 25° C. for 1 h. The reaction mixture was concentrated under reduced pressure to give (S)-tert-butyl ((6-(2,2′-dichloro-3′-(2-((((1-fluorocyclopropyl)methyl)amino)methyl)-3-methyl-4-oxo-3,4-dihydropyrrolo[2,1-f][1,2,4]triazin-6-yl)-[1,1′-biphenyl]-3-yl)-2-methoxypyridin-3-yl)methyl)((5-oxopyrrolidin-2-yl)methyl)carbamate as a white solid (0.2 g, crude, m/z: 804.3[M+H]⁺ observed).

### (S)-6-(2,2′-Dichloro-3′-(6-methoxy-5-((((5-oxopyrrolidin-2-yl)methyl)amino)methyl)pyridin-2-yl)-[1,1′-biphenyl]-3-yl)-2-((((1-fluorocyclopropyl)methyl)amino)methyl)-3-methylpyrrolo[2,1-f][1,2,4]triazin-4(3H)-one

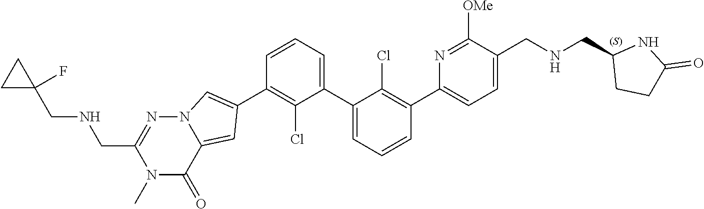

To a solution of (S)-tert-butyl ((6-(2,2′-dichloro-3′-(2-((((1-fluorocyclopropyl)methyl)amino) methyl)-3-methyl-4-oxo-3,4-dihydropyrrolo[2,1-f][1,2,4]triazin-6-yl)-[1,1′-biphenyl]-3-yl)-2-methoxypyridin-3-yl)methyl)((5-oxopyrrolidin-2-yl)methyl)carbamate (0.2 g, 0.19 mmol) in DCM (1 mL) was added TFA (1.3 mL). The mixture was stirred at 25° C. for 0.5 h. The reaction mixture was concentrated and the residue was purified by reverse phase HPLC to afford (S)-6-(2,2′-dichloro-3′-(6-methoxy-5-((((5-oxopyrrolidin-2-yl)methyl)amino) methyl)pyridin-2-yl)-[1,1′-biphenyl]-3-yl)-2-((((1-fluorocyclopropyl)methyl) amino)methyl)-3-methylpyrrolo[2,1-f][1,2,4]triazin-4(3H)-one as a white solid (34 mg, 25% yield, m/z: 704.3[M+H]⁺ observed). ¹H NMR (400 MHz, Methanol-d₄) δ 7.83-7.87 (m, 2H), 7.66 (dd, J=1.6 Hz, J=7.2 Hz, 2H), 7.44-7.54 (m, 2H), 7.39 (m, 1H), 7.28-7.35 (m, 3H), 3.98-4.20 (m, 8H), 3.61 (s, 3H), 3.13-3.19 (m, 2H), 2.97-3.03 (m, 2H), 2.35-2.45 (m, 3H), 1.86-1.92 (m, 1H), 0.99-1.12 (m, 2H), 0.77 (q, J=7.2 Hz, 2H).

### Example 480: (S)-6-(2,2′-Dichloro-3′-(6-methoxy-5-((((5-oxopyrrolidin-2-yl)methyl)amino)methyl)pyridin-2-yl)-[1,1′-biphenyl]-3-yl)-2-((3-hydroxy-3-methylazetidin-1-yl)methyl)-3-methylpyrrolo [2,1-f][1,2,4]triazin-4(3H)-one

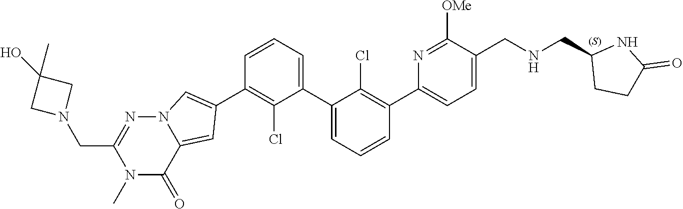

### (S)-tert-Butyl ((6-(2,2′-dichloro-3′-(2-((3-hydroxy-3-methylazetidin-1-yl)methyl)-3-methyl-4-oxo-3,4-dihydropyrrolo[2,1-f][1,2,4]triazin-6-yl)-[1,1′-biphenyl]-3-yl)-2-methoxypyridin-3-yl)methyl)((5-oxopyrrolidin-2-yl)methyl)carbamate

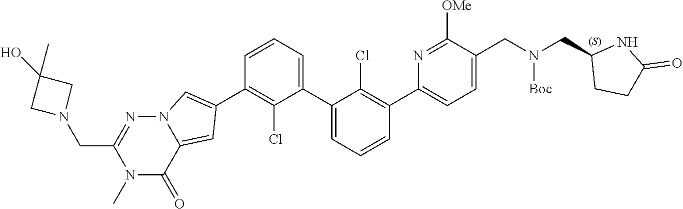

To a solution of (S)-tert-butyl ((6-(2,2′-dichloro-3′-(2-formyl-3-methyl-4-oxo-3,4-dihydropyrrolo [2,1-f][1,2,4]triazin-6-yl)-[1,1′-biphenyl]-3-yl)-2-methoxypyridin-3-yl)methyl)((5-oxopyrrolidin-2-yl)methyl)carbamate (0.1 g, 0.15 mmol) in DCM (4 mL) was added 4A molecular sieves (0.5 g), 3-methylazetidin-3-ol, HCl (0.025 g, 0.20 mmol) and sodium acetate (0.02 g, 0.25 mmol). The mixture was stirred at 25° C. for 11 h. Sodium cyanoborohydride (0.015 g, 0.25 mmol) was added and the mixture was stirred at 25° C. for 1 h. The reaction mixture was concentrated under reduced pressure to give (S)-tert-butyl ((6-(2,2′-dichloro-3′-(2-((3-hydroxy-3-methylazetidin-1-yl)methyl)-3-methyl-4-oxo-3,4-dihydropyrrolo[2,1-f][1,2,4]triazin-6-yl)-[1,1′-biphenyl]-3-yl)-2-methoxypyridin-3-yl)methyl)((5-oxopyrrolidin-2-yl)methyl)carbamate as a white solid (0.12 g, crude, m/z: 802.3 [M+H]⁺ observed).

### (S)-6-(2,2′-Dichloro-3′-(6-methoxy-5-((((5-oxopyrrolidin-2-yl)methyl)amino)methyl)pyridin-2-yl)-[1,1′-biphenyl]-3-yl)-2-((3-hydroxy-3-methylazetidin-1-yl)methyl)-3-methylpyrrolo [2,1-f][1,2,4]triazin-4(3H)-one

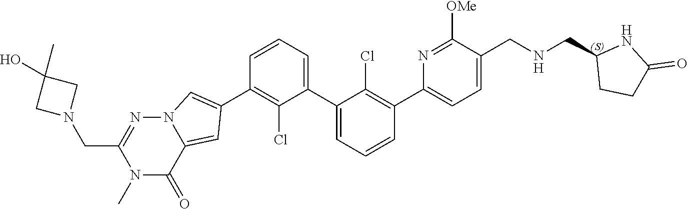

To a solution of (S)-tert-butyl ((6-(2,2′-dichloro-3′-(2-((3-hydroxy-3-methylazetidin-1-yl)methyl)-3-methyl-4-oxo-3,4-dihydropyrrolo[2,1-f][1,2,4]triazin-6-yl)-[1,1′-biphenyl]-3-yl)-2-methoxypyridin-3-yl)methyl)((5-oxopyrrolidin-2-yl)methyl)carbamate (0.12 g, 0.15 mmol) in DCM (30 mL) was added trifluoroacetic acid (1 mL). The mixture was stirred at 25° C. for 0.5 h. The reaction mixture was concentrated and the residue was purified by reverse phase HPLC to afford (S)-6-(2,2′-dichloro-3′-(6-methoxy-5-((((5-oxopyrrolidin-2-yl)methyl)amino)methyl) pyridin-2-yl)-[1,1′-biphenyl]-3-yl)-2-((3-hydroxy-3-methylazetidin-1-yl)methyl)-3-methylpyrrolo[2,1-f][1,2,4]triazin-4(3H)-one as a white solid (9.50 mg, 8% yield, m/z: 702.2 [M+H]⁺ observed). ¹H NMR (400 MHz, Methanol-d₄): δ 7.87-7.83 (m, 2H), 7.67-7.64 (m, 2H), 7.54-7.45 (m, 2H), 7.39 (dd, J=7.6 Hz J=1.6 Hz, 1H), 7.34 (d, J=7.6 Hz, 1H), 7.30-7.28 (m, 2H), 4.14 (m, 2H), 4.09 (s, 3H), 4.00-3.96 (m, 3H), 3.64-3.59 (m, 5H), 3.41-3.36 (m, 2H), 3.03-2.98 (m, 2H), 2.42-2.35 (m, 3H), 1.94-1.85 (m, 1H), 1.52 (s, 3H).

### Example 473: (S)-6-(2,2′-Dichloro-3′-(6-methoxy-5-((((5-oxopyrrolidin-2-yl)methyl)amino)methyl)pyridin-2-yl)-[1,1′-biphenyl]-3-yl)-2-(((3-hydroxybicyclo[1.1.1]pentan-1-yl)amino)methyl)-3-methylpyrrolo[2,1-f][1,2,4]triazin-4(3H)-one

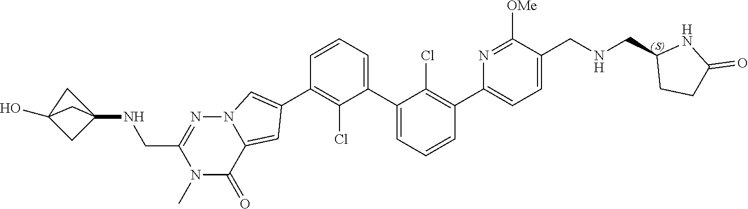

### (S)-tert-Butyl ((6-(2,2′-dichloro-3′-(2-(((3-hydroxybicyclo[1.1.1]pentan-1-yl)amino)methyl)-3-methyl-4-oxo-3,4-dihydropyrrolo[2,1-f][1,2,4]triazin-6-yl)-[1,1′-biphenyl]-3-yl)-2-methoxypyridin-3-yl)methyl)((5-oxopyrrolidin-2-yl)methyl)carbamate

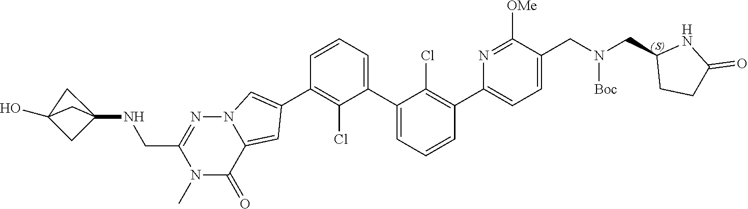

To a mixture of (S)-tert-butyl ((6-(2,2′-dichloro-3′-(2-formyl-3-methyl-4-oxo-3,4-dihydropyrrolo [2,1-f][1,2,4]triazin-6-yl)-[1,1′-biphenyl]-3-yl)-2-methoxypyridin-3-yl)methyl)((5-oxopyrrolidin-2-yl)methyl)carbamate (0.13 g, 0.18 mmol) and 3-aminobicyclo[1.1.1]pentan-1-ol, HCl (0.03 g, 0.25 mmol) in CH₂Cl₂ (4 mL) was added sodium acetate (0.044 g, 0.53 mmol). The mixture was stirred at rt for 7 h. Sodium triacetoxyborohydride (0.11 g, 0.53 mmol) was added and the mixture was stirred at rt for 8 h. The mixture was concentrated to afford (S)-tert-butyl ((6-(2,2′-dichloro-3′-(2-(((3-hydroxybicyclo[1.1.1]pentan-1-yl)amino)methyl)-3-methyl-4-oxo-3,4-dihydropyrrolo[2,1-f][1,2,4]triazin-6-yl)-[1,1′-biphenyl]-3-yl)-2-methoxypyridin-3-yl)methyl)((5-oxopyrrolidin-2-yl)methyl)carbamate as a light yellow solid (0.25 g, crude, m/z: 814 [M+H]⁺).

### (S)-6-(2,2′-Dichloro-3′-(6-methoxy-5-((((5-oxopyrrolidin-2-yl)methyl)amino)methyl)pyridin-2-yl)-[1,1′-biphenyl]-3-yl)-2-(((3-hydroxybicyclo[1.1.1]pentan-1-yl)amino)methyl)-3-methylpyrrolo[2,1-f][1,2,4]triazin-4(3H)-one

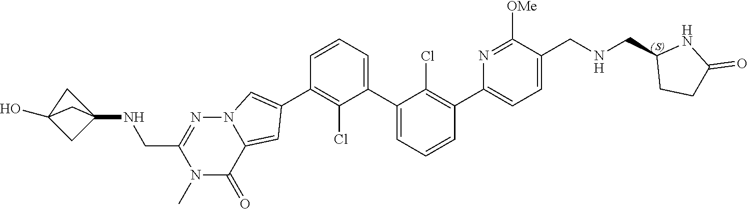

To a mixture of (S)-tert-butyl ((6-(2,2′-dichloro-3′-(2-(((3-hydroxybicyclo[1.1.1]pentan-1-yl)amino)methyl)-3-methyl-4-oxo-3,4-dihydropyrrolo[2,1-f][1,2,4]triazin-6-yl)-[1,1′-biphenyl]-3-yl)-2-methoxypyridin-3-yl)methyl)((5-oxopyrrolidin-2-yl)methyl)carbamate (0.25 g, 0.31 mmol) in CH₂Cl₂ (1 mL) was added trifluoroacetic acid (2 mL, 27 mmol). The mixture was stirred at rt for 0.5 h. The mixture was concentrated and the residue was purified by reverse phase HPLC to afford (S)-6-(2,2′-dichloro-3′-(6-methoxy-5-((((5-oxopyrrolidin-2-yl)methyl)amino) methyl)pyridin-2-yl)-[1,1′-biphenyl]-3-yl)-2-(((3-hydroxybicyclo[1.1.1]pentan-1-yl)amino) methyl)-3-methylpyrrolo[2,1-f][1,2,4]triazin-4(3H)-one as a gray solid (24 mg, 10% yield, m/z: 714 [M+H]⁺ observed). ¹H NMR (400 MHz, DMSO-d₆): δ 7.98 (d, J=2 Hz, 1H), 7.84 (d, J=7.6 Hz, 1H), 7.73 (d, J=8 Hz, J=1.6 Hz, 1H), 7.68-7.66 (m, 2H), 7.57-7.47 (m, 2H), 7.43 (d, J=7.6 Hz, J=1.6 Hz, 1H), 7.34 (d, J=7.6 Hz, J=1.6 Hz, 1H), 7.29 (d, J=7.6 Hz, 1H), 7.24 (d, J=1.6 Hz, 1H), 6.01 (s, 1H), 3.93 (s, 3H), 3.76 (s, 2H), 3.72 (s, 2H), 3.68-3.66 (m, 1H), 3.52 (s, 3H), 2.60 (d, J=5.6 Hz, 2H), 2.18-2.08 (m, 3H), 1.79 (s, 6H), 1.76-1.68 (m, 1H).

### Example 490: (S)-6-(2,2′-Dichloro-3′-(4-chloro-6-methoxy-5-((((5-oxopyrrolidin-2-yl)methyl)amino) methyl)pyridin-2-yl)-[1,1′-biphenyl]-3-yl)-2-(((3-fluoropropyl)amino)methyl)-3-methylpyrrolo[2,1-f][1,2,4]triazin-4(3H)-one

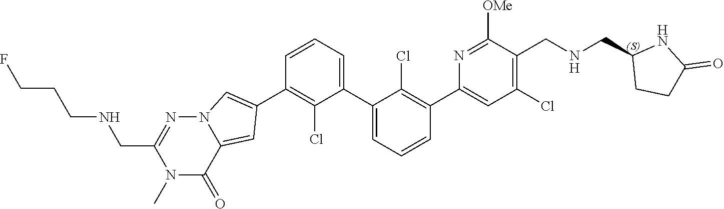

### (S)-tert-Butyl ((4-chloro-6-(2,2′-dichloro-3′-(2-(((3-fluoropropyl)amino)methyl)-3-methyl-4-oxo-3,4-dihydropyrrolo[2,1-f][1,2,4]triazin-6-yl)-[1,1′-biphenyl]-3-yl)-2-methoxypyridin-3-yl)methyl)((5-oxopyrrolidin-2-yl)methyl)carbamate

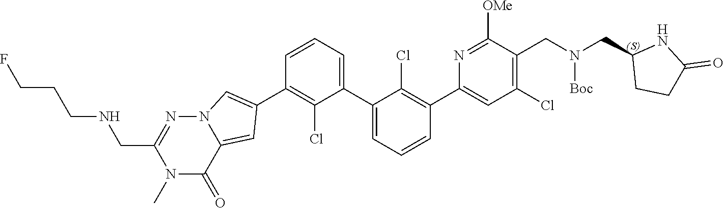

To a mixture of (S)-tert-butyl ((4-chloro-6-(2,2′-dichloro-3′-(2-formyl-3-methyl-4-oxo-3,4-dihydropyrrolo[2,1-f1][1,2,4]triazin-6-yl)-[1,1′-biphenyl]-3-yl)-2-methoxypyridin-3-yl)methyl)((5-oxopyrrolidin-2-yl)methyl)carbamate (200 mg, 0.26 mmol) and 3-fluoropropan-1-amine, HCl (50 mg, 0.44 mmol) in DCM/MeOH (1:1, 6 mL) was added sodium acetate (64.2 mg, 0.78 mmol,). The mixture was stirred at rt for 2 h under N₂. Sodium cyanoborohydride (49.2 mg, 0.78 mmol) was added and the mixture was stirred at rt for 0.5 h under N₂. The mixture was concentrated and the residue was purified by prep-TLC (SiO₂, EtOAc:MeOH=5:1) to afford (S)-tert-butyl ((4-chloro-6-(2,2′-dichloro-3′-(2-(((3-fluoropropyl)amino)methyl)-3-methyl-4-oxo-3,4-dihydropyrrolo[2,1-f][1,2,4]triazin-6-yl)-[1,1′-biphenyl]-3-yl)-2-methoxypyridin-3-yl)methyl)((5-oxopyrrolidin-2-yl)methyl)carbamate (110 mg, m/z: 826 [M+H]⁺, observed) as a white solid.

### (S)-6-(2,2′-Dichloro-3′-(4-chloro-6-methoxy-5-((((5-oxopyrrolidin-2-yl)methyl)amino) methyl)pyridin-2-yl)-[1,1′-biphenyl]-3-yl)-2-(((3-fluoropropyl)amino)methyl)-3-methylpyrrolo[2,1-f][1,2,4]triazin-4(3H)-one

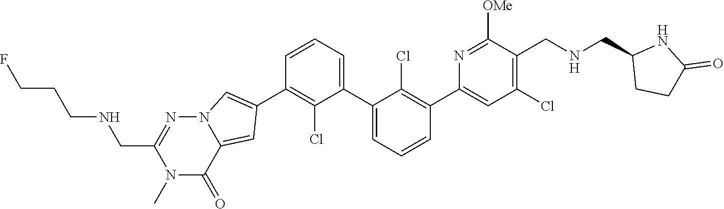 [SMILES: COc1nc(-c2cccc(-c3cccc(-c4cc5c(=O)n(C)c(CNCCCF)nn5c4)c3Cl)c2Cl)cc(Cl)c1CNC[C@@H]1CCC(=O)N1]

To a mixture of (S)-tert-butyl ((4-chloro-6-(2,2′-dichloro-3′-(2-(((3-fluoropropyl)amino)methyl)-3-methyl-4-oxo-3,4-dihydropyrrolo[2,1-f][1,2,4]triazin-6-yl)-[1,1′-biphenyl]-3-yl)-2-methoxypyridin-3-yl)methyl)((5-oxopyrrolidin-2-yl)methyl)carbamate (100 mg, 0.12 mmol) in DCM (5 mL) was added TFA (2.0 mL, 27 mmol). The mixture was stirred at rt for 0.5 h. The mixture was concentrated and the residue was purified by reverse phase HPLC to afford (S)-6-(2,2′-dichloro-3′-(4-chloro-6-methoxy-5-((((5-oxopyrrolidin-2-yl)methyl)amino)methyl)pyridin-2-yl)-[1,1′-biphenyl]-3-yl)-2-(((3-fluoropropyl)amino)methyl)-3-methylpyrrolo[2,1-f][1,2,4]triazin-4(3H)-one (15.3 mg, 17% yield, m/z: 726 [M+H]⁺ observed) as a white solid. NMR (400 MHz, Methanol-d₄): δ 7.87 (d, J=1.6 Hz, 1H), 7.68-7.65 (m, 2H), 7.52 (t, J=7.6 Hz, 1H), 7.47 (t, J=7.6 Hz, 1H), 7.42-7.39 (m, 1H), 7.36 (s, 1H), 7.30-7.28 (m, 2H), 4.62 (t, J=6 Hz, 1H), 4.50 (t, J=6 Hz, 1H), 4.07 (s, 3H), 4.03 (s, 2H), 3.94 (s, 2H), 3.86-3.85 (m, 1H), 3.60 (s, 3H), 3.89 (t, J=6.8 Hz, 1H), 2.73-2.70 (m, 2H), 2.37-2.28 (m, 3H), 2.01-1.84 (m, 2H), 1.81 (m, 1H).

### Example 491: (S)-6-(2,2′-Dichloro-3′-(4-chloro-6-methoxy-5-((((5-oxopyrrolidin-2-yl)methyl)amino) methyl)pyridin-2-yl)-[1,1′-biphenyl]-3-yl)-2-(((2-hydroxyethyl)amino)methyl)-3-methylpyrrolo[2,1-f][1,2,4]triazin-4(3H)-one

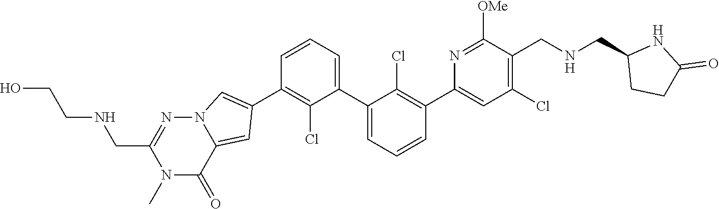 [SMILES: COc1nc(-c2cccc(-c3cccc(-c4cc5c(=O)n(C)c(CNCCO)nn5c4)c3Cl)c2Cl)cc(Cl)c1CNC[C@@H]1CCC(=O)N1]

### (S)-tert-Butyl ((4-chloro-6-(2,2′-dichloro-3′-(2-(((2-hydroxyethyl)amino)methyl)-3-methyl-4-oxo-3,4-dihydropyrrolo[2,1-f][1,2,4]triazin-6-yl)-[1,1′-biphenyl]-3-yl)-2-methoxypyridin-3-yl)methyl)((5-oxopyrrolidin-2-yl)methyl)carbamate

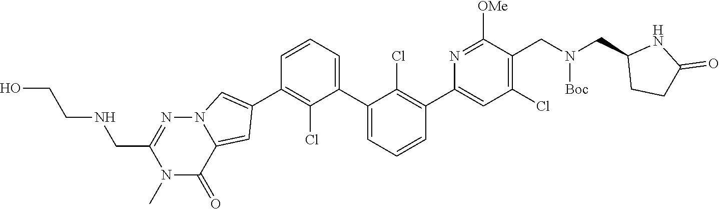 [SMILES: COc1nc(-c2cccc(-c3cccc(-c4cc5c(=O)n(C)c(CNCCO)nn5c4)c3Cl)c2Cl)cc(Cl)c1CN(C[C@@H]1CCC(=O)N1)C(=O)OC(C)(C)C]

To a solution of (S)-tert-butyl ((4-chloro-6-(2,2′-dichloro-3′-(2-formyl-3-methyl-4-oxo-3,4-dihydropyrrolo[2,1-f][1,2,4]triazin-6-yl)-[1,1′-biphenyl]-3-yl)-2-methoxypyridin-3-yl)methyl)((5-oxopyrrolidin-2-yl)methyl)carbamate (0.19 g, 0.25 mmol) in DCM (38 mL) was added 2-aminoethanol (0.038 g, 0.62 mmol). The mixture was stirred at 25° C. for 2 h. Sodium cyanoborohydride (0.03 g, 0.5 mmol) was added and the resulting mixture was stirred at 25° C. for 1 h. The reaction mixture was concentrated and the residue was purified by prep-TLC (SiO₂, EA:MeOH=3:1) to afford (S)-tert-butyl ((4-chloro-6-(2,2′-dichloro-3′-(2-(((2-hydroxyethyl) amino)methyl)-3-methyl-4-oxo-3,4-dihydropyrrolo[2,1-f][1,2,4]triazin-6-yl)-[1,1′-biphenyl]-3-yl)-2-methoxypyridin-3-yl)methyl)((5-oxopyrrolidin-2-yl)methyl)carbamate as a white solid (0.14 g, crude, m/z: 810.3[M+H]⁺ observed).

### (S)-6-(2,2′-Dichloro-3′-(4-chloro-6-methoxy-5-((((5-oxopyrrolidin-2-yl)methyl)amino) methyl)pyridin-2-yl)-[1,1′-biphenyl]-3-yl)-2-(((2-hydroxyethyl)amino)methyl)-3-methylpyrrolo[2,1-f][1,2,4]triazin-4(3H)-one

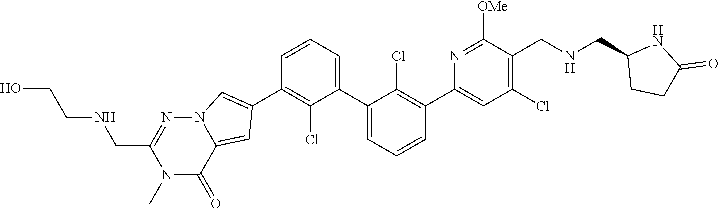 [SMILES: COc1nc(-c2cccc(-c3cccc(-c4cc5c(=O)n(C)c(CNCCO)nn5c4)c3Cl)c2Cl)cc(Cl)c1CNC[C@@H]1CCC(=O)N1]

To a solution of (S)-tert-butyl ((4-chloro-6-(2,2′-dichloro-3′-(2-(((2-hydroxyethyl) amino)methyl)-3-methyl-4-oxo-3,4-dihydropyrrolo[2,1-f][1,2,4]triazin-6-yl)-[1,1′-biphenyl]-3-yl)-2-methoxypyridin-3-yl)methyl)((5-oxopyrrolidin-2-yl)methyl)carbamate (0.12 g, 0.15 mmol) in DCM (5 mL) was added trifluoroacetic acid (5 mL). The mixture was stirred at 25° C. for 0.5 h. The reaction mixture was concentrated and the residue was purified by reverse phase HPLC to afford (S)-6-(2,2′-dichloro-3′-(4-chloro-6-methoxy-5-((((5-oxopyrrolidin-2-yl)methyl) amino)methyl)pyridin-2-yl)-[1,1′-biphenyl]-3-yl)-2-(((2-hydroxyethyl)amino)methyl)-3-methylpyrrolo[2,1-f][1,2,4]triazin-4(3H)-one as a white solid (15.2 mg, 11% yield, m/z: 710.2[M+H]⁺ observed). ¹H NMR (400 MHz, Methanol-d₄): δ 7.92 (d, J=1.6 Hz, 1H), 7.68-7.65 (m, 2H), 7.55-7.45 (m, 2H), 7.42-7.40 (m, 2H), 7.33-7.30 (m, 2H), 4.34 (s, 2H), 4.17-4.15 (m, 2H), 4.09 (s, 3H), 3.91-3.84 (m, 3H), 3.56 (s, 3H), 3.19 (br s, 2H), 2.90-2.87 (m, 2H), 2.42-2.30 (m, 3H), 1.88-1.81 (m, 1H).

### Example 492: (S)-6-(2,2′-Dichloro-3′-(4-chloro-6-methoxy-5-((((5-oxopyrrolidin-2-yl)methyl)amino) methyl)pyridin-2-yl)-[1,1′-biphenyl]-3-yl)-2-((3-hydroxy-3-methylazetidin-1-yl)methyl)-3-methylpyrrolo[2,1-f][1,2,4]triazin-4(3H)-one

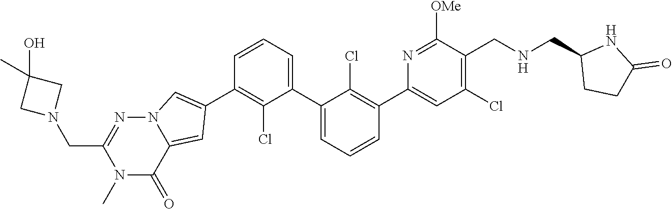 [SMILES: COc1nc(-c2cccc(-c3cccc(-c4cc5c(=O)n(C)c(CN6CC(C)(O)C6)nn5c4)c3Cl)c2Cl)cc(Cl)c1CNC[C@@H]1CCC(=O)N1]

### (S)-tert-Butyl ((4-chloro-6-(2,2′-dichloro-3′-(2-((3-hydroxy-3-methylazetidin-1-yl)methyl)-3-methyl-4-oxo-3,4-dihydropyrrolo[2,1-f][1,2,4]triazin-6-yl)-[1,1′-biphenyl]-3-yl)-2-methoxypyridin-3-yl)methyl)((5-oxopyrrolidin-2-yl)methyl)carbamate

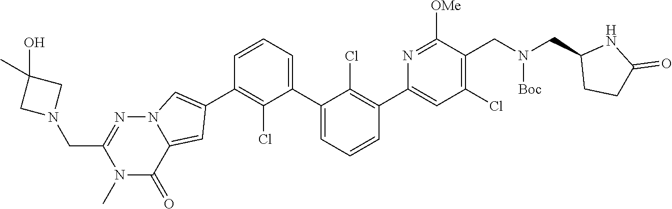 [SMILES: COc1nc(-c2cccc(-c3cccc(-c4cc5c(=O)n(C)c(CN6CC(C)(O)C6)nn5c4)c3Cl)c2Cl)cc(Cl)c1CN(C[C@@H]1CCC(=O)N1)C(=O)OC(C)(C)C]

To a mixture of (S)-tert-butyl ((4-chloro-6-(2,2′-dichloro-3′-(2-formyl-3-methyl-4-oxo-3,4-dihydropyrrolo[2,1-f][1,2,4]triazin-6-yl)-[1,1′-biphenyl]-3-yl)-2-methoxypyridin-3-yl)methyl) ((5-oxopyrrolidin-2-yl)methyl)carbamate (0.19 g, 0.25 mmol) and 3-methylazetidin-3-ol, HCl (0.06 g, 0.50 mmol) in DCM (30 mL) and MeOH (1 mL) was added sodium acetate (0.06 g, 0.74 mmol). The mixture was stirred at 25° C. for 11.5 h under N₂ atmosphere. Sodium cyanoborohydride (0.03 g, 0.50 mmol) was added and the mixture was stirred at 25° C. for 0.5 h. The reaction mixture was filtered and concentrated under reduced pressure to give a residue. The residue was purified by prep-TLC to give a (S)-tert-butyl ((4-chloro-6-(2,2′-dichloro-3′-(2-((3-hydroxy-3-methylazetidin-1-yl)methyl)-3-methyl-4-oxo-3,4-dihydropyrrolo[2,1-f][1,2,4]triazin-6-yl)-[1,1′-biphenyl]-3-yl)-2-methoxypyridin-3-yl)methyl)((5-oxopyrrolidin-2-yl)methyl)carbamate as a yellow solid (0.21 g, 89% yield, m/z: 836.2 [M+H]⁺ observed).

### (S)-6-(2,2′-Dichloro-3′-(4-chloro-6-methoxy-5-((((5-oxopyrrolidin-2-yl)methyl)amino) methyl)pyridin-2-yl)-[1,1′-biphenyl]-3-yl)-2-((3-hydroxy-3-methylazetidin-1-yl)methyl)-3-methylpyrrolo[2,1-f][1,2,4]triazin-4(3H)-one

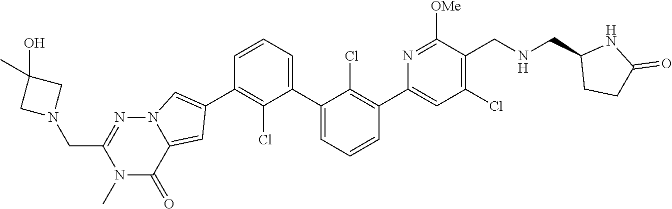 [SMILES: COc1nc(-c2cccc(-c3cccc(-c4cc5c(=O)n(C)c(CN6CC(C)(O)C6)nn5c4)c3Cl)c2Cl)cc(Cl)c1CNC[C@@H]1CCC(=O)N1]

To a mixture of (S)-tert-butyl ((4-chloro-6-(2,2′-dichloro-3′-(2-((3-hydroxy-3-methylazetidin-1-yl)methyl)-3-methyl-4-oxo-3,4-dihydropyrrolo[2,1-f][1,2,4]triazin-6-yl)-[1,1′-biphenyl]-3-yl)-2-methoxypyridin-3-yl)methyl)((5-oxopyrrolidin-2-yl)methyl)carbamate (0.20 g, 0.24 mmol) in DCM (10 mL) was added trifluoroacetic acid (1 mL). The mixture was stirred at 25° C. for 0.5 h. The reaction mixture was concentrated under reduced pressure and the residue was purified by reverse phase HPLC to afford (S)-6-(2,2′-dichloro-3′-(4-chloro-6-methoxy-5-((((5-oxopyrrolidin-2-yl)methyl)amino)methyl)pyridin-2-yl)-[1,1′-biphenyl]-3-yl)-2-((3-hydroxy-3-methylazetidin-1-yl)methyl)-3-methylpyrrolo[2,1-f][1,2,4]triazin-4(3H)-one as a white solid (61.9 mg, m/z: 736.2 [M+H]⁺ observed). ¹H NMR (400 MHz, Methanol-d₄): δ 7.87 (d, J=2.0 Hz, 1H), 7.68-7.65 (m, 2H), 7.53 (t, J=7.6 Hz, 1H), 7.47 (t, J=7.6 Hz, 1H), 7.42-7.40 (m, 2H), 7.30-7.29 (m, 2H), 4.59 (m, 1H), 4.16 (s, 2H), 4.09 (s, 3H), 4.00 (m, 2H), 3.91 (m, 1H), 3.67-3.65 (m, 3H), 3.60 (s, 3H) 3.50-3.43 (m, 1H), 2.89-2.86 (m, 2 H), 2.40-2.30 (m, 3H), 1.88-1.83 (m, 1H), 1.53 (s, 3H)

### Example 499: (S)-6-(2,2′-Dichloro-3′-(4-fluoro-6-methoxy-5-((((5-oxopyrrolidin-2-yl)methyl)amino) methyl)pyridin-2-yl)-[1,1′-biphenyl]-3-yl)-2-((3-hydroxy-3-methylazetidin-1-yl)methyl)-3-methylpyrrolo[2,1-f][1,2,4]triazin-4(3H)-one

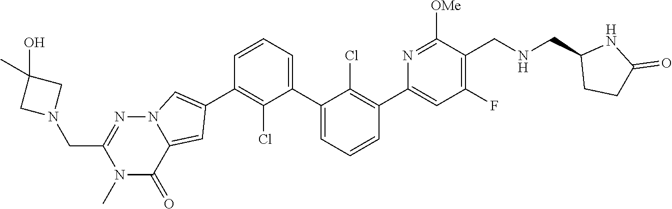 [SMILES: COc1nc(-c2cccc(-c3cccc(-c4cc5c(=O)n(C)c(CN6CC(C)(O)C6)nn5c4)c3Cl)c2Cl)cc(F)c1CNC[C@@H]1CCC(=O)N1]

### (S)-tert-Butyl ((6-(3′-bromo-2,2′-dichloro-[1,1′-biphenyl]-3-yl)-4-fluoro-2-methoxypyridin-3-yl)methyl)((5-oxopyrrolidin-2-yl)methyl)carbamate

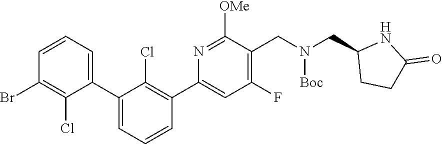 [SMILES: COc1nc(-c2cccc(-c3cccc(Br)c3Cl)c2Cl)cc(F)c1CN(C[C@@H]1CCC(=O)N1)C(=O)OC(C)(C)C]

To a mixture of 6-(3′-bromo-2,2′-dichloro-[1,1′-biphenyl]-3-yl)-4-fluoro-2-methoxynicotinaldehyde (2 g, 4.4 mmol) and (S)-5-(aminomethyl)pyrrolidin-2-one, HCl (0.8 g, 5.3 mmol) in DCM (15 mL) was added sodium acetate (1.1 g, 13.2 mmol). The was stirred at rt for 12 h. Sodium triacetyloxyboranuide (1.86 g, 8.8 mmol) was added and the mixture was stirred at rt for 2 h under N₂. To this mixture was added di-tert-butyl dicarbonate (2.2 g, 10 mmol) and triethylamine (1.7 mL, 12 mmol). The mixture was stirred at rt for 1.5 h under N₂. The mixture was filtered and the filtrate was concentrated. The residue was purified by normal phase SiO₂ chromatography (0-65% ethyl acetate/petroleum ether) to afford (S)-tert-butyl ((6-(3′-bromo-2,2′-dichloro-[1,1′-biphenyl]-3-yl)-4-fluoro-2-methoxypyridin-3-yl)methyl)((5-oxopyrrolidin-2-yl)methyl)carbamate (2.2 g, 84% yield) as a yellow solid. ¹H NMR (400 MHz, DMSO-d₆): δ7.68 (d, J=7.6 Hz, 1H), 7.78 (s, 1H), 7.71-7.68 (m, 1H), 7.56 (t, J=7.2 Hz, 1H), 7.46-7.40 (m, 3H), 7.23 (d, J=10 Hz, 1H), 4.54-4.39 (m, 2H), 3.97 (s, 3H), 3.77-3.76 (m, 1H), 3.33-3.30 (m, 2H), 2.16-2.06 (m, 3H), 1.72-1.64 (m, 1H), 1.40-1.28 (m, 9H).

### (S)-tert-Butyl ((6-(2,2′-dichloro-3′-(4,4,5,5-tetramethyl-1,3,2-dioxaborolan-2-yl)-[1,1′-biphenyl]-3-yl)-4-fluoro-2-methoxypyridin-3-yl)methyl)((5-oxopyrrolidin-2-yl)methyl)carbamate

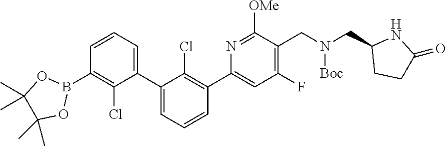 [SMILES: COc1nc(-c2cccc(-c3cccc(B4OC(C)(C)C(C)(C)O4)c3Cl)c2Cl)cc(F)c1CN(C[C@@H]1CCC(=O)N1)C(=O)OC(C)(C)C]

To a mixture of (S)-tert-butyl ((6-(3′-bromo-2,2′-dichloro-[1,1′-biphenyl]-3-yl)-4-fluoro-2-methoxypyridin-3-yl)methyl)((5-oxopyrrolidin-2-yl)methyl)carbamate (1.7 g, 2.6 mmol) and bis(pinacolato)diboron (2 g, 7.8 mmol) in dioxane (30 mL) was added potassium acetate (0.64 g, 6.5 mmol) and 1,1′-bis(diphenylphosphino)ferrocene-palladium(II) dichloride dichloromethane complex (0.1 g, 0.13 mmol). The mixture was stirred at 110° C. for 6 h. The mixture was filtered, and the filtrate was concentrated. The residue was purified by normal phase SiO₂ chromatography (0-90% ethyl acetate/petroleum ether) to afford (S)-tert-butyl ((6-(2,2′-dichloro-3′-(4,4,5,5-tetramethyl-1,3,2-dioxaborolan-2-yl)-[1,1′-biphenyl]-3-yl)-4-fluoro-2-methoxypyridin-3-yl)methyl)((5-oxopyrrolidin-2-yl)methyl)carbamate (1.5 g, 82% yield) as a yellow solid. ¹H NMR (400 MHz, CDCl₃): δ7.64-7.62 (d, J=5.2 Hz, 1H), 7.59-7.57 (m, 1H), 7.35-7.33 (m, 1H), 7.52-7.22 (m, 2H), 7.21-7.20 (m, 1H), 7.03 (d, J=9.6 Hz, 1H), 4.05-4.45 (m, 2H), 3.96 (s, 3H), 3.78-3.75 (br s, 1H), 3.39-3.19 (m, 2H), 2.26-2.08 (m, 3H), 1.71-1.66 (m, 1H), 1.43-1.41 (m, 21H).

### (S)-tert-Butyl ((6-(2,2′-dichloro-3′-(2-(hydroxymethyl)-3-methyl-4-oxo-3,4-dihydropyrrolo [2,1-f][1,2,4]triazin-6-yl)-[1,1′-biphenyl]-3-yl)-4-fluoro-2-methoxypyridin-3-yl)methyl)((5-oxopyrrolidin-2-yl)methyl)carbamate

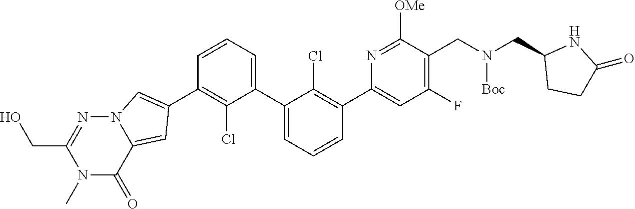 [SMILES: COc1nc(-c2cccc(-c3cccc(-c4cc5c(=O)n(C)c(CO)nn5c4)c3Cl)c2Cl)cc(F)c1CN(C[C@@H]1CCC(=O)N1)C(=O)OC(C)(C)C]

A mixture of (S)-tert-butyl ((6-(2,2′-dichloro-3′-(4,4,5,5-tetramethyl-1,3,2-dioxaborolan-2-yl)-[1,1′-biphenyl]-3-yl)-4-fluoro-2-methoxypyridin-3-yl)methyl)((5-oxopyrrolidin-2-yl)methyl)carbamate (1.7 g, 2.4 mmol), 6-bromo-2-(hydroxymethyl)-3-methyl-pyrrolo[2,1-f][1,2,4]triazin-4-one (0.48 g, 1.9 mmol), [1,1′-bis(di-tert-butylphosphino)ferrocene]dichloropalladium(II) (60 mg, 93 umol), potassium phosphate (1.2 g, 5.6 mmol) in dioxane/water mixture (5:1, 60 mL) was stirred at 110° C. for 0.5 h. The mixture was concentrated and the residue was purified by normal phase SiO₂ chromatography (0-100% ethyl acetate/petroleum ether) to afford (S)-tert-butyl ((6-(2,2′-dichloro-3′-(2-(hydroxymethyl)-3-methyl-4-oxo-3,4-dihydropyrrolo[2,1-f][1,2,4]triazin-6-yl)-[1,1′-biphenyl]-3-yl)-4-fluoro-2-methoxypyridin-3-yl)methyl)((5-oxopyrrolidin-2-yl)methyl)carbamate (1.2 g, 85% yield) as a yellow solid.

### (S)-tert-Butyl ((6-(2,2′-dichloro-3′-(2-formyl-3-methyl-4-oxo-3,4-dihydropyrrolo[2,1-f][1,2,4]triazin-6-yl)-[1,1′-biphenyl]-3-yl)-4-fluoro-2-methoxypyridin-3-yl)methyl)((5-oxopyrrolidin-2-yl)methyl)carbamate

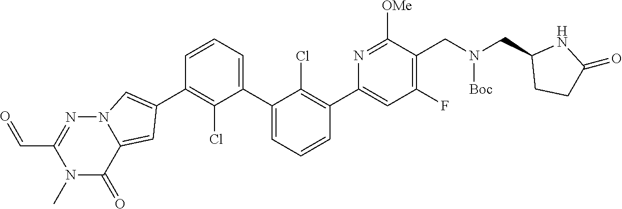 [SMILES: COc1nc(-c2cccc(-c3cccc(-c4cc5c(=O)n(C)c(C=O)nn5c4)c3Cl)c2Cl)cc(F)c1CN(C[C@@H]1CCC(=O)N1)C(=O)OC(C)(C)C]

To a mixture of (S)-tert-butyl ((6-(2,2′-dichloro-3′-(2-(hydroxymethyl)-3-methyl-4-oxo-3,4-dihydropyrrolo[2,1-f]][1,2,4]triazin-6-yl)-[1,1′-biphenyl]-3-yl)-4-fluoro-2-methoxypyridin-3-yl)methyl)((5-oxopyrrolidin-2-yl)methyl)carbamate (1.2 g, 1.60 mmol) in DCM (30 mL) was added Dess-Martin periodinane (0.88 g, 2.1 mmol). The mixture was stirred at rt for 0.5 h under N₂. The mixture was filtered, and the filtrate was concentrated. The residue was purified by normal phase SiO₂ chromatography (0-100% ethyl acetate/petroleum ether) to afford (S)-tert-butyl ((6-(2,2′-dichloro-3′-(2-formyl-3-methyl-4-oxo-3,4-dihydropyrrolo[2,1-f1][1,2,4]triazin-6-yl)-[1,1′-biphenyl]-3-yl)-4-fluoro-2-methoxypyridin-3-yl)methyl)((5-oxopyrrolidin-2-yl)methyl)carbamate (1 g, 83% yield) as a yellow solid. ¹H NMR (400 MHz, CDCl₃): δ9.56 (s, 1H), 7.89 (s, 1H), 7.65 (d, J=7.2 Hz, 1H), 7.56 (d, J=7.2 Hz, 1H), 7.44-7.35 (m, 4H), 7.33-7.30 (m, 1H), 7.28-7.24 (m, 1H), 7.10 (d, J=9.6 Hz, 1H), 7.56-7.50 (m, 2H), 4.03-4.01 (m, 4H), 3.83-3.80 (m, 5H), 3.36-3.30 (m, 2H), 2.33-2.13 (m, 3H), 1.77-1.75 (m, 1H), 1.45-1.42 (m, 9H).

### (S)-tert-Butyl ((6-(2,2′-dichloro-3′-(2-((3-hydroxy-3-methylazetidin-1-yl)methyl)-3-methyl-4-oxo-3,4-dihydropyrrolo[2,1-f][1,2,4]triazin-6-yl)-[1,1′-biphenyl]-3-yl)-4-fluoro-2-methoxypyridin-3-yl)methyl)((5-oxopyrrolidin-2-yl)methyl)carbamate

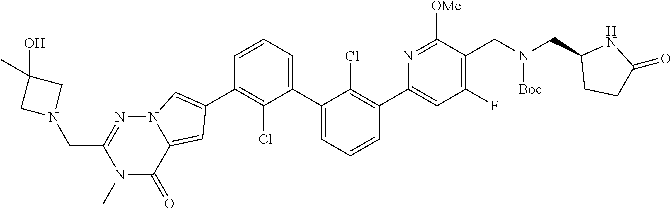 [SMILES: COc1nc(-c2cccc(-c3cccc(-c4cc5c(=O)n(C)c(CN6CC(C)(O)C6)nn5c4)c3Cl)c2Cl)cc(F)c1CN(C[C@@H]1CCC(=O)N1)C(=O)OC(C)(C)C]

To a mixture of (S)-tert-butyl ((6-(2,2′-dichloro-3′-(2-formyl-3-methyl-4-oxo-3,4-dihydropyrrolo [2,1-f][1,2,4]triazin-6-yl)-[1,1′-biphenyl]-3-yl)-4-fluoro-2-methoxypyridin-3-yl)methyl)((5-oxopyrrolidin-2-yl)methyl)carbamate (0.4 g, 0.53 mmol) and 3-methylazetidin-3-ol, HCl (0.1 g, 0.8 mmol) in DCM/trimethoxymethane mixture (4:1, 10 mL) was added sodium acetate (0.11 g, 1.3 mmol). The mixture was stirred at rt for 4 h under N₂. Sodium cyanoborohydride (84 mg, 1.33 mmol) was added and the mixture was stirred at rt for 0.5 h. The mixture was diluted with water (15 mL) and extracted with DCM (2×45 mL). The combined organic phase was dried with anhydrous sodium sulfate, filtered and concentrated. The residue was purified by prep-TLC (SiO₂, ethyl acetate:MeOH=10:1) to afford (S)-tert-butyl ((6-(2,2′-dichloro-3′-(2-((3-hydroxy-3-methylazetidin-1-yl)methyl)-3-methyl-4-oxo-3,4-dihydropyrrolo[2,1-f1][1,2,4]triazin-6-yl)-[1,1′-biphenyl]-3-yl)-4-fluoro-2-methoxypyridin-3-yl)methyl)((5-oxopyrrolidin-2-yl)methyl)carbamate (0.13 g, 29% yield, m/z: 820 [(M+H]⁺observed) as a yellow solid.

### (S)-6-(2,2′-Dichloro-3′-(4-fluoro-6-methoxy-5-((((5-oxopyrrolidin-2-yl)methyl)amino) methyl)pyridin-2-yl)-[1,1′-biphenyl]-3-yl)-2-((3-hydroxy-3-methylazetidin-1-yl)methyl)-3-methylpyrrolo[2,1-f][1,2,4]triazin-4(3H)-one

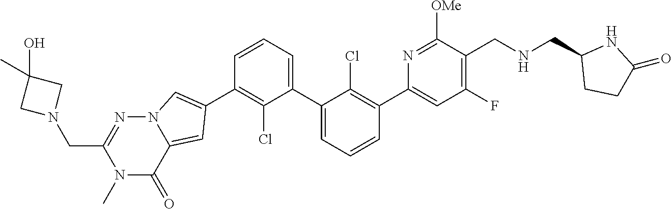 [SMILES: COc1nc(-c2cccc(-c3cccc(-c4cc5c(=O)n(C)c(CN6CC(C)(O)C6)nn5c4)c3Cl)c2Cl)cc(F)c1CNC[C@@H]1CCC(=O)N1]

To a solution of (S)-tert-butyl ((6-(2,2′-dichloro-3′-(2-((3-hydroxy-3-methylazetidin-1-yl)methyl)-3-methyl-4-oxo-3,4-dihydropyrrolo[2,1-f][1,2,4]triazin-6-yl)-[1,1′-biphenyl]-3-yl)-4-fluoro-2-methoxypyridin-3-yl)methyl)((5-oxopyrrolidin-2-yl)methyl)carbamate (0.13 g, 0.16 mmol) in DCM (2 mL) was added trifluoroacetic acid (3 mL). The mixture was stirred at rt for 0.5 h. The mixture was concentrated and the residue was purified by reverse phase HPLC to afford (S)-6-(2,2′-dichloro-3′-(4-fluoro-6-methoxy-5-((((5-oxopyrrolidin-2-yl)methyl)amino) methyl)pyridin-2-yl)-[1,1′-biphenyl]-3-yl)-2-((3-hydroxy-3-methylazetidin-1-yl)methyl)-3-methylpyrrolo[2,1-f][1,2,4]triazin-4(3H)-one (20.4 mg, 16% yield, m/z: 720 [(M+H]⁺ observed) as a white solid. ¹H NMR (400 MHz, Methanol-d₄): δ8.29 (s, 1H), 7.83 (s, 1H), 7.63 (t, J=8.8 Hz, 2H), 7.49 (t, J=7.6 Hz, 1H), 7.43 (t, J=7.6 Hz, 1H), 7.38 (d, J=8 Hz, 1H), 7.26-7.24 (m, 2H), 7.16 (d, J=9.6 Hz, 1H), 4.06-4.99 (m, 5H), 3.89 (s, 2H), 3.92-3.86 (m, 1H), 3.64 (d, J=8 Hz, 2H), 3.55 (s, 3H), 3.43 (d, J=7.6 Hz, 2H), 2.91-2.83 (m, 2H), 3.89-2.27 (m, 3H), 1.85-1.78 (m, 1H), 1.49 (s, 3H).

### Example 500: (S)-6-(2,2′-Dichloro-3′-(4-fluoro-6-methoxy-5-((((5-oxopyrrolidin-2-yl)methyl)amino) methyl)pyridin-2-yl)-[1,1′-biphenyl]-3-yl)-2-(((2-hydroxyethyl)amino)methyl)-3-methylpyrrolo[2,1-f][1,2,4]triazin-4(3H)-one

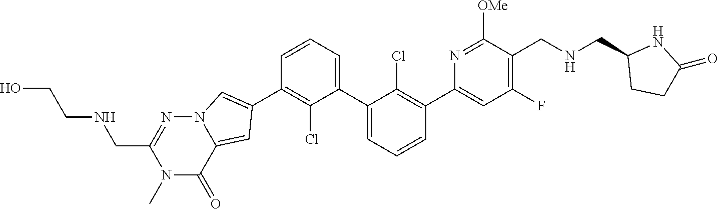 [SMILES: COc1nc(-c2cccc(-c3cccc(-c4cc5c(=O)n(C)c(CNCCO)nn5c4)c3Cl)c2Cl)cc(F)c1CNC[C@@H]1CCC(=O)N1]

### (S)-tert-Butyl ((6-(2,2′-dichloro-3′-(2-(((2-hydroxyethyl)amino)methyl)-3-methyl-4-oxo-3,4-dihydropyrrolo[2,1-f][1,2,4]triazin-6-yl)-[1,1′-biphenyl]-3-yl)-4-fluoro-2-methoxypyridin-3-yl)methyl)((5-oxopyrrolidin-2-yl)methyl)carbamate

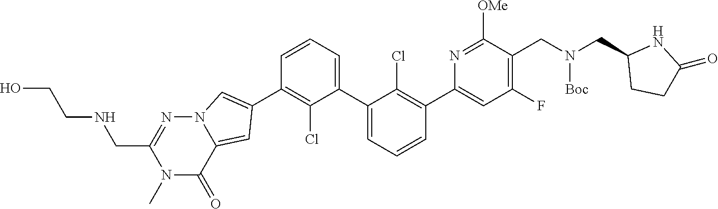 [SMILES: COc1nc(-c2cccc(-c3cccc(-c4cc5c(=O)n(C)c(CNCCO)nn5c4)c3Cl)c2Cl)cc(F)c1CN(C[C@@H]1CCC(=O)N1)C(=O)OC(C)(C)C]

To a mixture of (S)-tert-butyl ((6-(2,2′-dichloro-3′-(2-formyl-3-methyl-4-oxo-3,4-dihydropyrrolo[2,1-f][1,2,4]triazin-6-yl)-[1,1′-biphenyl]-3-yl)-4-fluoro-2-methoxypyridin-3-yl)methyl)((5-oxopyrrolidin-2-yl)methyl)carbamate (0.3 g, 0.4 mmol) and 2-aminoethanol (48 uL, 0.8 mmol) in DCM (10 mL) was added sodium acetate (66 mg, 0.8 mmol). The mixture was stirred at rt for 11.5 h. Sodium triacetyloxyboranuide (0.17 g, 0.8 mmol) was added and the mixture was stirred at rt for 1.5 h. The mixture was filtered, and the filtrate was concentrated to give the residue. The residue was purified by prep-TLC (SiO₂, EtOAc:MeOH=10:1) to afford (S)-tert-butyl ((6-(2,2′-dichloro-3′-(2-(((2-hydroxyethyl)amino)methyl)-3-methyl-4-oxo-3,4-dihydropyrrolo[2,1-f][1,2,4]triazin-6-yl)-[1,1′-biphenyl]-3-yl)-4-fluoro-2-methoxypyridin-3-yl)methyl)((5-oxopyrrolidin-2-yl)methyl)carbamate (0.15 g, 47% yield) as a yellow solid.

### (S)-6-(2,2′-Dichloro-3′-(4-fluoro-6-methoxy-5-((((5-oxopyrrolidin-2-yl)methyl)amino) methyl)pyridin-2-yl)-[1,1′-biphenyl]-3-yl)-2-(((2-hydroxyethyl)amino)methyl)-3-methylpyrrolo[2,1-f][1,2,4]triazin-4(3H)-one

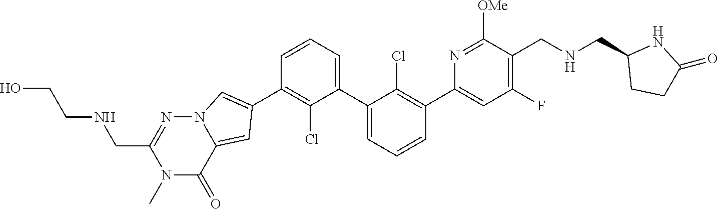 [SMILES: COc1nc(-c2cccc(-c3cccc(-c4cc5c(=O)n(C)c(CNCCO)nn5c4)c3Cl)c2Cl)cc(F)c1CNC[C@@H]1CCC(=O)N1]

To a solution of (S)-tert-butyl ((6-(2,2′-dichloro-3′-(2-(((2-hydroxyethyl)amino)methyl)-3-methyl-4-oxo-3,4-dihydropyrrolo[2,1-f][1,2,4]triazin-6-yl)-[1,1′-biphenyl]-3-yl)-4-fluoro-2-methoxypyridin-3-yl)methyl)((5-oxopyrrolidin-2-yl)methyl)carbamate (0.15 g, 0.2 mmol) in DCM (2 mL) was added trifluoroacetic acid (3 mL). The mixture was stirred at rt for 0.5 h. The mixture was concentrated and the residue was purified by reverse phase HPLC to afford (S)-6-(2,2′-dichloro-3′-(4-fluoro-6-methoxy-5-((((5-oxopyrrolidin-2-yl)methyl)amino)methyl)pyridin-2-yl)-[1,1′-biphenyl]-3-yl)-2-(((2-hydroxyethyl)amino)methyl)-3-methylpyrrolo[2,1-f][1,2,4] triazin-4(3H)-one (29.4 mg, 22% yield, m/z: 694 [(M+H]⁺ observed) as a white solid. ¹H NMR (400 MHz, Methanol-d₄): δ7.88 (s, 1H), 7.65-7.62 (m, 2H), 7.51-7.42 (m, 2H), 7.37 (d, J=7.2 Hz, 1H), 7.29-7.25 (m, 2H), 7.15 (m, 6.8 Hz, 1H), 4.26 (s, 2H), 4.06 (s, 3H), 4.01 (s, 2H), 3.87-3.79 (m, 3H), 3.52 (s, 3H), 3.13-3.11 (m, 2H), 2.84-2.78 (m, 2H), 2.38-2.26 (m, 3H), 1.48-1.79 (m, 1H).

### Example 473: (S)-6-(2,2′-Dichloro-3′-(6-methoxy-5-((((5-oxopyrrolidin-2-yl)methyl)amino)methyl)pyridin-2-yl)-[1,1′-biphenyl]-3-yl)-2-(((3-hydroxybicyclo[1.1.1]pentan-1-yl)amino)methyl)-3-methylpyrrolo[2,1-f][1,2,4]triazin-4(3H)-one

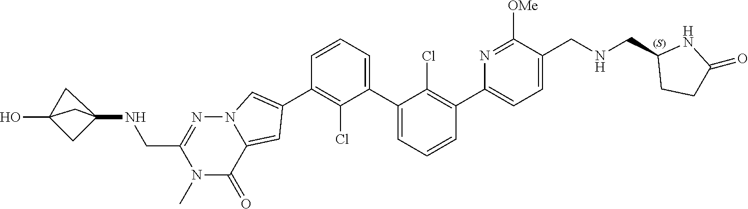

### (S)-tert-butyl ((6-(2,2′-Dichloro-3′-(2-(((3-hydroxybicyclo[1.1.1]pentan-1-yl)amino)methyl)-3-methyl-4-oxo-3,4-dihydropyrrolo[2,1-f][1,2,4]triazin-6-yl)-[1,1′-biphenyl]-3-yl)-2-methoxypyridin-3-yl)methyl)((5-oxopyrrolidin-2-yl)methyl)carbamate

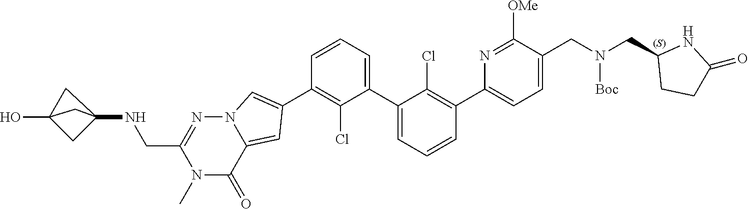

To a mixture of (S)-tert-butyl ((6-(2,2′-dichloro-3′-(2-formyl-3-methyl-4-oxo-3,4-dihydropyrrolo[2,1-f1][1,2,4]triazin-6-yl)-[1,1′-biphenyl]-3-yl)-2-methoxypyridin-3-yl)methyl) ((5-oxopyrrolidin-2-yl)methyl)carbamate (0.13 g, 0.18 mmol) and 3-aminobicyclo[1.1.1]pentan-1-ol, HCl (0.03 g, 0.25 mmol) in CH₂Cl₂ (4 mL) was added sodium acetate (0.044 g, 0.53 mmol) in one portion under N₂ atmosphere. The mixture was stirred at rt for 7 h. Sodium triacetoxyborohydride (0.11 g, 0.53 mmol) was added and the mixture was stirred at rt for 8 h. The mixture was concentrated to afford (S)-tert-butyl ((6-(2,2′-dichloro-3′-(2-(((3-hydroxybicyclo[1.1.1]pentan-1-yl)amino)methyl)-3-methyl-4-oxo-3,4-dihydropyrrolo[2,1-f][1,2,4]triazin-6-yl)-[1,1′-biphenyl]-3-yl)-2-methoxypyridin-3-yl)methyl)((5-oxopyrrolidin-2-yl)methyl)carbamate as a light-yellow solid. The material was uses further without any purification (0.25 g, crude, m/z: 814 [M+H]⁺ observed).

### (S)-6-(2,2′-Dichloro-3′-(6-methoxy-5-((((5-oxopyrrolidin-2-yl)methyl)amino)methyl)pyridin-2-yl)-[1,1′-biphenyl]-3-yl)-2-(((3-hydroxybicyclo[1.1.1]pentan-1-yl)amino)methyl)-3-methylpyrrolo[2,1-f][1,2,4]triazin-4(3H)-one

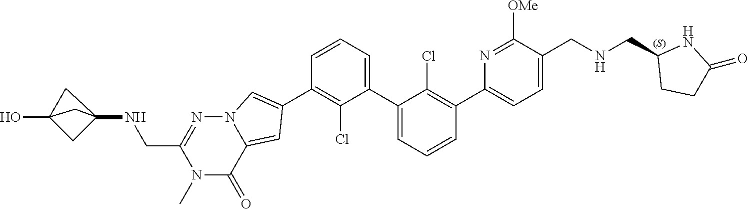

To a mixture of (S)-tert-butyl ((6-(2,2′-dichloro-3′-(2-(((3-hydroxybicyclo[1.1.1]pentan-1-yl)amino)methyl)-3-methyl-4-oxo-3,4-dihydropyrrolo[2,1-f][1,2,4]triazin-6-yl)-[1,1′-biphenyl]-3-yl)-2-methoxypyridin-3-yl)methyl)((5-oxopyrrolidin-2-yl)methyl)carbamate (0.25 g, 0.31 mmol) in CH₂Cl₂ (1 mL) was added trifluoroacetic acid (2 mL, 27 mmol) in one portion. The mixture was stirred at rt for 0.5 h. The mixture was concentrated and the residue was purified by reverse phase HPLC to afford (S)-6-(2,2′-dichloro-3′-(6-methoxy-5-((((5-oxopyrrolidin-2-yl)methyl)amino)methyl)pyridin-2-yl)-[1,1′-biphenyl]-3-yl)-2-(((3-hydroxybicyclo[1.1.1]pentan-1-yl)amino)methyl)-3-methylpyrrolo[2,1-f][1,2,4]triazin-4(3H)-one as a gray solid (24.3 mg, 10% yield, m/z: 714 [M+H]⁺ observed). ¹H NMR (400 MHz, DMSO-d₆): δ 7.98 (d, J=2 Hz, 1H), 7.84 (d, J=7.6 Hz, 1H), 7.73 (d, J=8 Hz, J=1.6 Hz, 1H), 7.68-7.66 (m, 2H), 7.57-7.47 (m, 2H), 7.43 (d, J=7.6 Hz, J=1.6 Hz, 1H), 7.34 (d, J=7.6 Hz, J=1.6 Hz, 1H), 7.29 (d, J=7.6 Hz, 1H), 7.24 (d, J=1.6 Hz, 1H), 6.01 (s, 1H), 3.93 (s, 3H), 3.76 (s, 2H), 3.72 (s, 2H), 3.68-3.66 (m, 1H), 3.52 (s, 3H), 2.60 (d, J=5.6 Hz, 2H), 2.18-2.08 (m, 3H), 1.79 (s, 6H), 1.76-1.68 (m, 1H).

### Example 480: 6-(2,2′-Dichloro-3′-(4-fluoro-6-methoxy-5-(((((S)-5-oxopyrrolidin-2-yl)methyl)amino) methyl)pyridin-2-yl)-[1,1′-biphenyl]-3-yl)-2-((((1r,3r)-3-hydroxy-3-methylcyclobutyl) amino)methyl)-3-methylpyrrolo[2,1-f][1,2,4]triazin-4(3H)-one

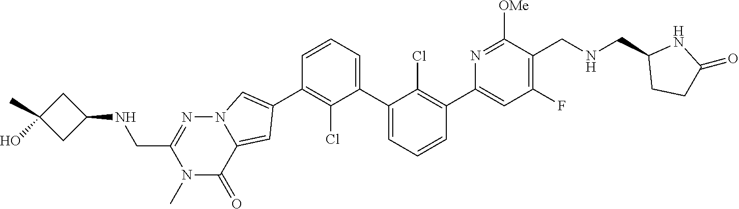 [SMILES: COc1nc(-c2cccc(-c3cccc(-c4cc5c(=O)n(C)c(CN[C@H]6C[C@](C)(O)C6)nn5c4)c3Cl)c2Cl)cc(F)c1CNC[C@@H]1CCC(=O)N1]

### (S)-5-((((6-(3′-Bromo-2,2′-dichloro-[1,1′-biphenyl]-3-yl)-4-fluoro-2-methoxypyridin-3-yl)methyl)amino)methyl)pyrrolidin-2-one

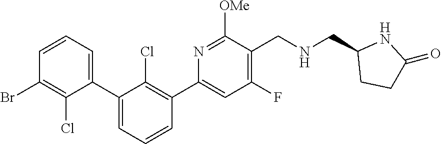 [SMILES: COc1nc(-c2cccc(-c3cccc(Br)c3Cl)c2Cl)cc(F)c1CNC[C@@H]1CCC(=O)N1]

A mixture of 6-(3′-bromo-2,2′-dichloro-[1,1′-biphenyl]-3-yl)-4-fluoro-2-methoxynicotinaldehyde (0.30 g, 0.66 mmol), (S)-5-(aminomethyl)pyrrolidin-2-one, HCl (0.20 g, 1.32 mmol) and sodium acetate (0.24 g, 2.97 mmol) in DCM (5 mL) was degassed and purged with N₂, and stirred at rt for 12 h. Sodium triacetoxyborohydride (0.35 g, 1.65 mmol) was added and the mixture was stirred at rt for 2 h. The solvent was evaporated to afford (S)-5-((((6-(3′-bromo-2,2′-dichloro-[1,1′-biphenyl]-3-yl)-4-fluoro-2-methoxypyridin-3-yl)methyl)amino)methyl)pyrrolidin-2-one. The crude product was used as is without any purification. (m/z: 554.0 [M+H]⁺ observed).

### (S)-tert-Butyl ((6-(3′-bromo-2,2′-dichloro-[1,1′-biphenyl]-3-yl)-4-fluoro-2-methoxypyridin-3-yl)methyl)((5-oxopyrrolidin-2-yl)methyl)carbamate

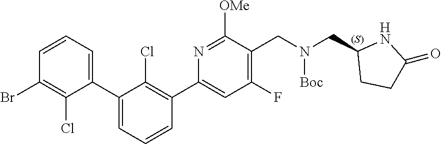

To a solution of (S)-5-((((6-(3′-bromo-2,2′-dichloro-[1,1′-biphenyl]-3-yl)-4-fluoro-2-methoxypyridin-3-yl)methyl)amino)methyl)pyrrolidin-2-one (0.66 mmol) in DCM (5 mL) was added triethylamine (0.23 mL) and Boc₂O (0.38 mL). The mixture was stirred at rt for 40 min under N₂. The reaction mixture was diluted with water (20 mL) and the aqueous layer was extracted with EtOAc (3×15 mL). The combined organic layers were washed with brine (2×10 mL), dried over anhydrous sodium sulfate, filtered and concentrated. The residue was purified by normal phase SiO₂ chromatography (0-100% EtOAc/petroleum ether) to afford (S)-tert-butyl ((6-(3′-bromo-2,2′-dichloro-[1,1′-biphenyl]-3-yl)-4-fluoro-2-methoxypyridin-3-yl)methyl)((5-oxopyrrolidin-2-yl)methyl)carbamate as a brown solid (0.35 g, 66% yield, m/z:654.1[M+H]⁺ observed).

### 2-(Hydroxymethyl)-3-methyl-6-(4,4,5,5-tetramethyl-1,3,2-dioxaborolan-2-yl)pyrrolo[2,1-f][1,2,4]triazin-4(3H)-one

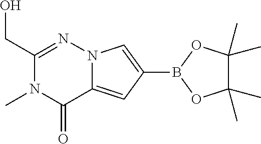

To a mixture of 6-bromo-2-(hydroxymethyl)-3-methylpyrrolo[2,1-f][1,2,4]triazin-4(3H)-one (0.50 g, 1.94 mmol) and 4,4,4′,4′,5,5,5′,5′-octamethyl-2,2′-bi(1,3,2-dioxaborolane) (2.46 g, 9.69 mmol) in dioxane (15 mL) was added potassium acetate (0.38 g, 3.87 mmol) and 1,1′-bis(diphenylphosphino)ferrocene-palladium(ii) dichloride dichloromethane complex (0.16 g, 0.19 mmol). The mixture was stirred at 110° C. for 3 h under N₂. The mixture was filtered, and the filtrate was concentrated. The residue was purified by normal phase SiO₂ chromatography (0-100% EtOAc/petroleum ether) to afford 2-(hydroxymethyl)-3-methyl-6-(4,4,5,5-tetramethyl-1,3,2-dioxaborolan-2-yl)pyrrolo[2,1-f][1,2,4]triazin-4(3H)-one as a yellow solid (0.39 g, 65% yield). ¹H NMR (400 MHz, DMSO-d₆) δ 7.66 (d, J=1.2 Hz, 1H), 6.95 (d, J=1.6 Hz, 1H), 4.44 (d, J=5.6 Hz, 3H), 3.46-3.45 (m, 4H), 1.24 (s, 12H).

### (S)-tert-Butyl ((6-(2,2′-dichloro-3′-(2-(hydroxymethyl)-3-methyl-4-oxo-3,4-dihydropyrrolo [2,1-f][1,2,4]triazin-6-yl)-[1,1′-biphenyl]-3-yl)-4-fluoro-2-methoxypyridin-3-yl)methyl)((5-oxopyrrolidin-2-yl)methyl)carbamate

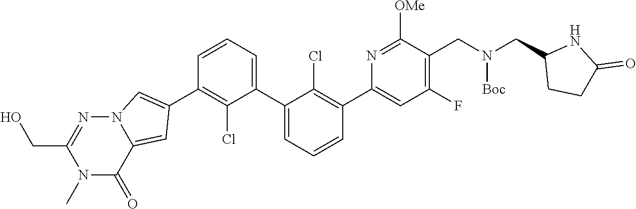

To a mixture of (S)-tert-butyl ((6-(3′-bromo-2,2′-dichloro-[1,1′-biphenyl]-3-yl)-4-fluoro-2-methoxypyridin-3-yl)methyl)((5-oxopyrrolidin-2-yl)methyl)carbamate (0.29 g, 0.44 mmol) and 2-(hydroxymethyl)-3-methyl-6-(4,4,5,5-tetramethyl-1,3,2-dioxaborolan-2-yl)pyrrolo[2,1-f][1,2,4]triazin-4(3H)-one (0.19 g, 0.62 mmol) in dioxane (5 mL) and water (0.5 mL) was added 1,1′-bis(diphenylphosphino)ferrocene-palladium(II)dichloride dichloromethane complex (0.05 g, 0.07 mmol) and potassium carbonate (0.03 g, 0.22 mmol). The mixture was stirred at 110° C. for 0.5 h. The reaction mixture was diluted with water (20 mL) and the aqueous layer was extracted with EtOAc (3×15 mL). The combined organic layers were washed with brine (2×10 mL), dried over anhydrous sodium sulfate, filtered and concentrated. The residue was purified by normal phase SiO₂ chromatography (0-100% EtOAc/petroleum ether) to afford (S)-tert-butyl ((6-(2,2′-dichloro-3′-(2-(hydroxymethyl)-3-methyl-4-oxo-3,4-dihydropyrrolo[2,1-f][1,2,4]triazin-6-yl)-[1,1′-biphenyl]-3-yl)-4-fluoro-2-methoxypyridin-3-yl)methyl)((5-oxopyrrolidin-2-yl)methyl)carbamate as a yellow solid (0.23 g, 64% yield). ¹H NMR (400 MHz, DMSO-d₆): δ 7.96 (d, J=1.6 Hz, 1H), 7.73 (s, 1H), 7.69 (dd, J=2.0 Hz, J=6.0 Hz, 1H),7.65-7.64 (m, 1H), 7.52 (t, J=7.6 Hz, 1H), 7.48-7.42 (m, 2 H), 7.30 (dd, J=1.6 Hz, J=6.0 Hz, 1H), 7.19 (m, 2H), 5.85-5.82 (m, 1H), 4.48-4.40 (m, 4 H), 3.91 (s, 3H), 3.73 (m, 1H), 3.48 (s, 3H), 2.10-2.00 (m, 3H), 1.66 (m, 1H), 1.34-1.24 (m, 9H).

### (S)-tert-Butyl ((6-(2,2′-dichloro-3′-(2-formyl-3-methyl-4-oxo-3,4-dihydropyrrolo[2,1-f][1,2,4]triazin-6-yl)-[1,1′-biphenyl]-3-yl)-4-fluoro-2-methoxypyridin-3-yl)methyl)((5-oxopyrrolidin-2-yl)methyl)carbamate

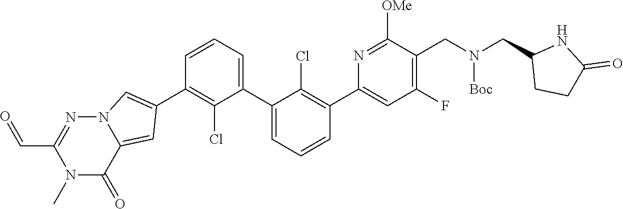

To a mixture of (S)-tert-butyl ((6-(2,2′-dichloro-3′-(2-(hydroxymethyl)-3-methyl-4-oxo-3,4-dihydropyrrolo[2,1-f1][1,2,4]triazin-6-yl)-[1,1′-biphenyl]-3-yl)-4-fluoro-2-methoxypyridin-3-yl)methyl)((5-oxopyrrolidin-2-yl)methyl)carbamate (0.21 g, 0.28 mmol) in DCM (5 mL) was added Dess-Martin (0.24 g, 0.56 mmol). The mixture was stirred at 25° C. for 1 h under N₂.

The reaction mixture was diluted with water (20 ml) and the aqueous layer was extracted with EtOAc (3×15 mL). The combined organic layers were washed with brine (2×10 mL), dried over anhydrous sodium sulfate, filtered and concentrated. The residue was purified by normal phase SiO₂ chromatography (0-100% EtOAc/petroleum ether) to afford (S)-tert-butyl ((6-(2,2′-dichloro-3′-(2-formyl-3-methyl-4-oxo-3,4-dihydropyrrolo[2,1-f][1,2,4]triazin-6-yl)-[1,1′-biphenyl]-3-yl)-4-fluoro-2-methoxypyridin-3-yl)methyl)((5-oxopyrrolidin-2-yl)methyl) carbamate as a yellow solid (0.28 g, m/z: 749.3 [M+H]⁺ observed).

### tert-Butyl ((6-(2,2′-dichloro-3′-(2-((((1r,3r)-3-hydroxy-3-methylcyclobutyl)amino)methyl)-3-methyl-4-oxo-3,4-dihydropyrrolo[2,1-f][1,2,4]triazin-6-yl)-[1,1′-biphenyl]-3-yl)-4-fluoro-2-methoxypyridin-3-yl)methyl)(((S)-5-oxopyrrolidin-2-yl)methyl)carbamate

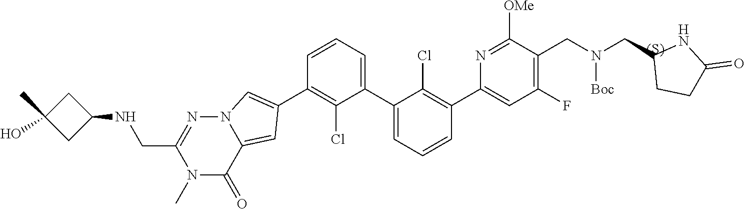

A mixture of (S)-tert-butyl ((6-(2,2′-dichloro-3′-(2-formyl-3-methyl-4-oxo-3,4-dihydropyrrolo [2,1-f][1,2,4]triazin-6-yl)-[1,1′-biphenyl]-3-yl)-4-fluoro-2-methoxypyridin-3-yl)methyl)((5-oxopyrrolidin-2-yl)methyl)carbamate (0.27 g, 0.36 mmol,), (1r,3r)-3-amino-1-methylcyclobutanol, HCl (0.12 g, 0.90 mmol) and sodium acetate (0.12 g, 1.44 mmol), 4A molecular sieve (20 mg) in DCM (15 mL) was degassed and purged with N₂. The mixture was stirred at 25° C. for 11.5 h under N₂ atmosphere. Sodium cyanoborohydride (0.07 g, 1.08 mmol) was added and the mixture was stirred at 25° C. for 0.5 h under N₂ atmosphere. The reaction mixture was filtered and concentrated under reduced pressure to give tert-butyl ((6-(2,2′-dichloro-3′-(2-((((1r,3r)-3-hydroxy-3-methylcyclobutyl)amino)methyl)-3-methyl-4-oxo-3,4-dihydropyrrolo[2,1-f1][1,2,4]triazin-6-yl)-[1,1′-biphenyl]-3-yl)-4-fluoro-2-methoxypyridin-3-yl)methyl)(((S)-5-oxopyrrolidin-2-yl)methyl)carbamate as a white solid (0.30 g, crude, m/z: 834.3 [M+H]⁺ observed).

### 6-(2,2′-Dichloro-3′-(4-fluoro-6-methoxy-5-(((((S)-5-oxopyrrolidin-2-yl)methyl)amino) methyl)pyridin-2-yl)-[1,1′-biphenyl]-3-yl)-2-((((1r,3r)-3-hydroxy-3-methylcyclobutyl) amino)methyl)-3-methylpyrrolo[2,1-f][1,2,4]triazin-4(3H)-one

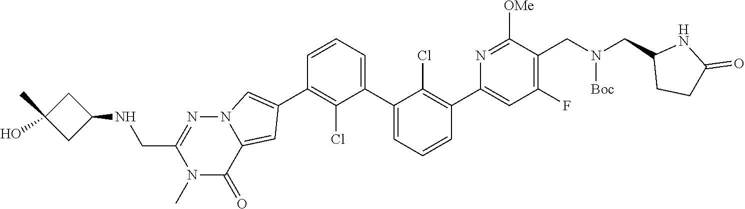

To a mixture of tert-butyl ((6-(2,2′-dichloro-3′-(2-((((1r,3r)-3-hydroxy-3-methylcyclobutyl) amino)methyl)-3-methyl-4-oxo-3,4-dihydropyrrolo[2,1-f][1,2,4]triazin-6-yl)-[1,1′-biphenyl]-3-yl)-4-fluoro-2-methoxypyridin-3-yl)methyl)(((S)-5-oxopyrrolidin-2-yl)methyl)carbamate (0.30 g, 0.36 mmol) in DCM was added trifluoroacetic acid (1.5 mL). The mixture was stirred at 25° C. for 0.5 h. The reaction mixture was concentrated under reduced pressure to give a residue. The residue was purified by reverse phase HPLC to afford 6-(2,2′-dichloro-3′-(4-fluoro-6-methoxy-5-(((((S)-5-oxopyrrolidin-2-yl)methyl)amino)methyl)pyridin-2-yl)-[1,1′-biphenyl]-3-yl)-2-((((1r,3r)-3-hydroxy-3-methylcyclobutyl)amino)methyl)-3-methylpyrrolo[2,1-f][1,2,4]triazin-4(3H)-one as a white solid (34 mg, m/z: 734.3[M+H]⁺ observed). ¹H NMR (400 MHz, DMSO-d₆): δ 7.99 (d, J=2.0 Hz, 1H), 7.74-7.69 (m, 2H), 7.64 (s, 1H), 7.57 (t, J=7.6 Hz, 1H), 7.52-7.46 (m, 2H), 7.34 (dd, J=1.2 Hz, J=6 Hz, 1H), 7.27-7.24 (m, 2H), 4.69 (m, 1H), 3.97 (s, 3H), 3.79-3.62 (m, 5H), 3.55 (s, 3H), 3.42 (m, 1H), 2.58 (m, 2H), 2.16-2.07 (m, 5H), 1.72-1.64 (m, 3H), 1.26 (s, 3H).

### Example 457: 2-[[(1-Acetyl-4-piperidyl)amino]methyl]-6-[2-chloro-3-[2-chloro-3-[6-methoxy-5-[[[(2S)-5-oxopyrrolidin-2-yl]methylamino]methyl]-2-pyridyl]phenyl]phenyl]-3-methyl-pyrrolo[2,1-f][1,2,4]triazin-4-one

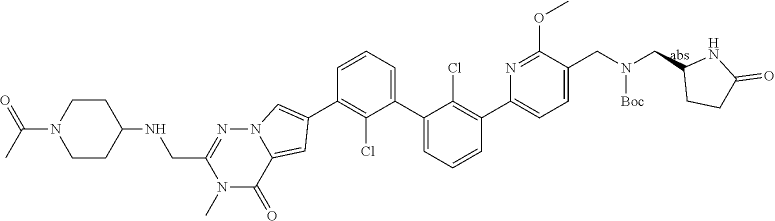

### tert-Butyl N-[[6-[3-[3-[2-[[(1-acetyl-4-piperidyl)amino]methyl]-3-methyl-4-oxo-pyrrolo[2,1-f][1,2,4]triazin-6-yl]-2-chloro-phenyl]-2-chloro-phenyl]-2-methoxy-3-pyridyl]methyl]-N-[[(2S)-5-oxopyrrolidin-2-yl]methyl]carbamate

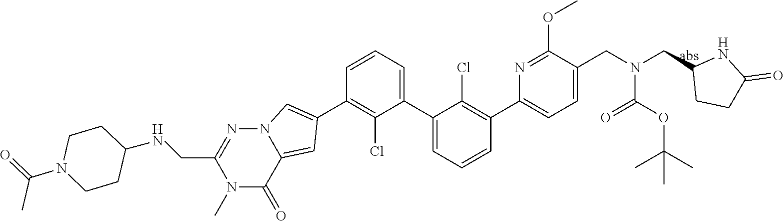

A mixture of tert-butyl N-[[6-[2-chloro-3-[2-chloro-3-(2-formyl-3-methyl-4-oxo-pyrrolo[2,1-f1][1,2,4]triazin-6-yl)phenyl]phenyl]-2-methoxy-3-pyridyl]methyl]-N-[[(2S)-5-oxopyrrolidin-2-yl]methyl]carbamate (35 mg, 0.05 mmol), 1-(4-amino-1-piperidyl)ethanone (14 mg, 0.10 mmol) and acetic acid (2.7 uL, 2.9 mg, 0.05 mmol) in MeOH/THF (1:1) was stirred for 1.5 hour at rt. NaBH₃CN (6 mg, 0.10 mmol) was added, the mixture was stirred for 30 min, quenched with water, directly purified by reversed phase chromatography to give tert-butyl N-[[6-[3-[3-[2-[[(1-acetyl-4-piperidyl)amino]methyl]-3-methyl-4-oxo-pyrrolo[2,1-f1][1,2,4]triazin-6-yl]-2-chloro-phenyl]-2-chloro-phenyl]-2-methoxy-3-pyridyl]methyl]-N-[[(2S)-5-oxopyrrolidin-2-yl]methyl]carbamate (36 mg, 88%). MS: m/z found 857.3 [M+H]⁺.

### 2-[[(1-Acetyl-4-piperidyl)amino]methyl]-6-[2-chloro-3-[2-chloro-3-[6-methoxy-5-[[[(2S)-5-oxopyrrolidin-2-yl]methylamino]methyl]-2-pyridyl]phenyl]phenyl]-3-methyl-pyrrolo[2,1-f][1,2,4]triazin-4-one

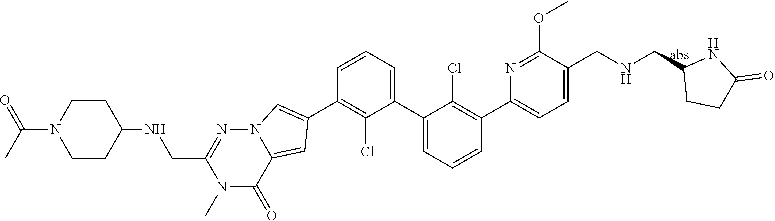

A solution of tert-butyl N-[[6-[3-[3-[2-[[(1-acetyl-4-piperidyl)amino]methyl]-3-methyl-4-oxo-pyrrolo[2,1-f][1,2,4]triazin-6-yl]-2-chloro-phenyl]-2-chloro-phenyl]-2-methoxy-3-pyridyl]methyl]-N-[[(2S)-5-oxopyrrolidin-2-yl]methyl]carbamate (13 mg, 0.02 mmol) in DCM/TFA (2:1) was stirred for 30 minutes, concentrated in vacuo and purified by reversed phase chromatography to give 2-[[(1-acetyl-4-piperidyl)amino]methyl]-6-[2-chloro-3-[2-chloro-3-[6-methoxy-5-[[[(2S)-5-oxopyrrolidin-2-yl]methylamino]methyl]-2-pyridyl]phenyl]phenyl]-3-methyl-pyrrolo[2,1-f][1,2,4]triazin-4-one (11 mg, 95.8%). MS: m/z found 757.3 [M+H]⁺. ¹H NMR (400 MHz, Methanol-d₄): δ 7.86 (d, J=1.9 Hz, 1H), 7.73 (d, J=7.5 Hz, 1H), 7.63 (dt, J=7.7, 1.7 Hz, 2H), 7.46 (dt, J=14.9, 7.6 Hz, 2H), 7.35 (dd, J=7.5, 1.7 Hz, 1H), 7.30-7.23 (m, 3H), 4.41 (d, J=13.5 Hz, 1H), 4.03 (s, 3H), 3.99-3.94 (m, 2H), 3.95-3.80 (m, 4H), 3.60 (s, 3H), 3.19 (ddd, J=14.1, 11.6, 2.8 Hz, 1H), 2.90 (td, J=10.1, 5.0 Hz, 1H), 2.84-2.76 (m, 1H), 2.70 (td, J=12.2, 6.8 Hz, 2H), 2.38-2.24 (m, 3H), 2.10 (s, 3H), 2.09-1.96 (m, 2H), 1.87-1.77 (m, 1H), 1.46-1.25 (m, 2H).

### Example 458: 6-[2-Chloro-3-[2-chloro-3-[6-methoxy-5-[[[(2S)-5-oxopyrrolidin-2-yl]methylamino]methyl]-2-pyridyl]phenyl]phenyl]-3-methyl-2-[[2-(2-oxopyrrolidin-1-yl)ethylamino]methyl]pyrrolo[2,1-f][1,2,4]triazin-4-one

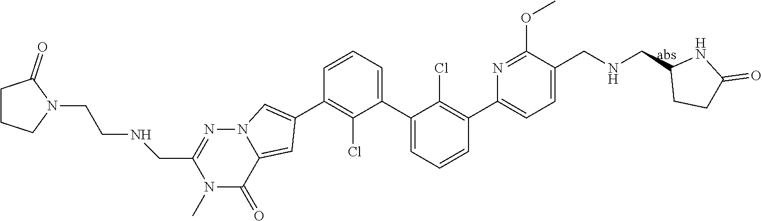

### tert-Butyl N-[[6-[2-chloro-3-[2-chloro-3-[3-methyl-4-oxo-2-[[2-(2-oxopyrrolidin-1-yl)ethylamino]methyl]pyrrolo[2,1-f][1,2,4]triazin-6-yl]phenyl]phenyl]-2-methoxy-3-pyridyl]methyl]-N-[[(2S)-5-oxopyrrolidin-2-yl]methyl]carbamate

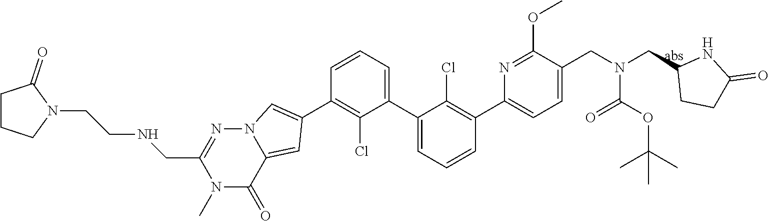

A mixture of tert-butyl N-[[6-[2-chloro-3-[2-chloro-3-(2-formyl-3-methyl-4-oxo-pyrrolo[2,1-f][1,2,4]triazin-6-yl)phenyl]phenyl]-2-methoxy-3-pyridyl]methyl]-N-[[(2S)-5-oxopyrrolidin-2-yl]methyl]carbamate (35 mg, 0.05 mmol), 1-(2-aminoethyl)pyrrolidin-2-one (12 mg, 0.10 mmol) and acetic acid (2.7 uL, 2.9 mg, 0.05 mmol) in MeOH/THF (1:1) was stirred for 1.5 hour at rt. NaBH₃CN (6.01 mg, 0.10 mmol) was added, the mixture was stirred for 30 min, quenched with water and directly purified by reversed phase chromatography to give tert-butyl N-[[6-[2-chloro-3-[2-chloro-3-[3-methyl-4-oxo-2-[[2-(2-oxopyrrolidin-1-yl)ethylamino]methyl]pyrrolo[2,1-f][1,2,4]triazin-6-yl]phenyl]phenyl]-2-methoxy-3-pyridyl]methyl]-N-[[(2S)-5-oxopyrrolidin-2-yl]methyl]carbamate (35 mg, 86.7%). MS: m/z found 843.3 [M+H]⁺.

### 6-[2-Chloro-3-[2-chloro-3-[6-methoxy-5-[[[(2S)-5-oxopyrrolidin-2-yl]methylamino]methyl]-2-pyridyl]phenyl]phenyl]-3-methyl-2-[[2-(2-oxopyrrolidin-1-yl)ethylamino]methyl]pyrrolo[2,1-f][1,2,4]triazin-4-one

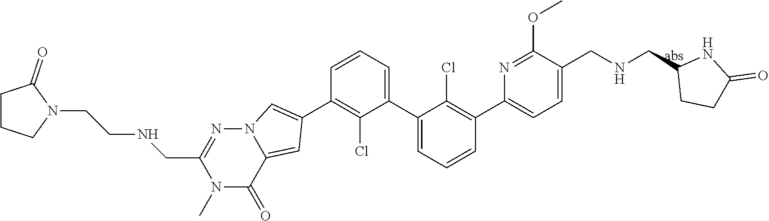

A solution of tert-butyl N-[[6-[2-chloro-3-[2-chloro-3-[3-methyl-4-oxo-2-[[2-(2-oxopyrrolidin-1-yl)ethylamino]methyl]pyrrolo[2,1-f][1,2,4]triazin-6-yl]phenyl]phenyl]-2-methoxy-3-pyridyl]methyl]-N-[[(2S)-5-oxopyrrolidin-2-yl]methyl]carbamate (12 mg, 0.01 mmol) in DCM/TFA (2:1) was stirred for 30 minutes, concentrated in vacuo and purified by reversed phase chromatography to give 6-[2-chloro-3-[2-chloro-3-[6-methoxy-5-[[[(2S)-5-oxopyrrolidin-2-yl]methylamino]methyl]-2-pyridyl]phenyl]phenyl]-3-methyl-2-[[2-(2-oxopyrrolidin-1-yl)ethylamino]methyl]pyrrolo[2,1-f][1,2,4]triazin-4-one (10 mg, 94.6%). MS: m/z found 743.3 [M+H]⁺. ¹H NMR (400 MHz, Methanol-d₄): δ 7.85 (d, J=1.9 Hz, 1H), 7.73 (d, J=7.5 Hz, 1H), 7.63 (ddd, J=7.7, 2.7, 1.7 Hz, 2H), 7.46 (dt, J=14.4, 7.7 Hz, 2H), 7.35 (dd, J=7.6, 1.7 Hz, 1H), 7.29-7.24 (m, 3H), 4.03 (s, 3H), 3.93 (s, 2H), 3.88-3.79 (m, 3H), 3.59 (s, 3H), 3.53-3.48 (m, 2H), 3.46 (t, J=6.2 Hz, 2H), 2.93 (t, J=6.2 Hz, 2H), 2.75-2.63 (m, 2H), 2.42-2.23 (m, 5H), 2.04 (p, J=7.7 Hz, 2H), 1.87-1.76 (m, 1H).

### Example 472: 6-[2-Chloro-3-[2-chloro-3-[6-methoxy-5-[[[(2S)-5-oxopyrrolidin-2-yl]methylamino]methyl]-2-pyridyl]phenyl]phenyl]-3-methyl-2-[[[(2S)-oxetan-2-yl]methylamino]methyl]pyrrolo[2,1-f][1,2,4]triazin-4-one

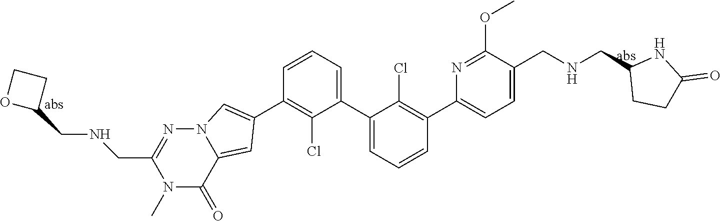

### tert-Butyl N-[[6-[2-chloro-3-[2-chloro-3-[3-methyl-2-[[[(2S)-oxetan-2-yl]methylamino]methyl]-4-oxo-pyrrolo[2,1-f]1[1,2,4]triazin-6-yl]phenyl]phenyl]-2-methoxy-3-pyridyl]methyl]-N-[[(2S)-5-oxopyrrolidin-2-yl]methyl]carbamate

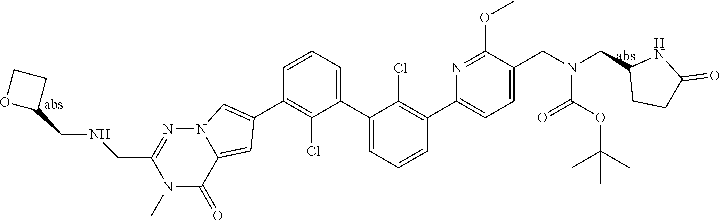

A mixture of tert-butyl N-[[6-[2-chloro-3-[2-chloro-3-(2-formyl-3-methyl-4-oxo-pyrrolo[2,1-f1][1,2,4]triazin-6-yl)phenyl]phenyl]-2-methoxy-3-pyridyl]methyl]-N-[[(2S)-5-oxopyrrolidin-2-yl]methyl]carbamate (30 mg, 0.04 mmol), [(2S)-oxetan-2-yl]methanamine (7 mg, 0.08 mmol) and acetic acid (2.4 uL, 2.5 mg, 0.04 mmol) in MeOH/THF (1:1) was stirred for 1.5 hour at rt. NaBH₃CN (5.15 mg, 0.08 mmol) was added, the mixture was stirred for 30 min, quenched with water and directly purified by reversed phase chromatography to give tert-butyl N-[[6-[2-chloro-3-[2-chloro-3-[3-methyl-2-[[[(2S)-oxetan-2-yl]methylamino]methyl]-4-oxo-pyrrolo[2,1-f][1,2,4]triazin-6-yl]phenyl]phenyl]-2-methoxy-3-pyridyl]methyl]-N-[[(2S)-5-oxopyrrolidin-2-yl]methyl]carbamate (25 mg, 76.0%). MS: m/z found 802.3 [M+H]⁺.

### 6-[2-Chloro-3-[2-chloro-3-[6-methoxy-5-[[[(2S)-5-oxopyrrolidin-2-yl]methylamino]methyl]-2-pyridyl]phenyl]phenyl]-3-methyl-2-[[[(2S)-oxetan-2-yl]methylamino]methyl]pyrrolo[2,1-f][1,2,4]triazin-4-one

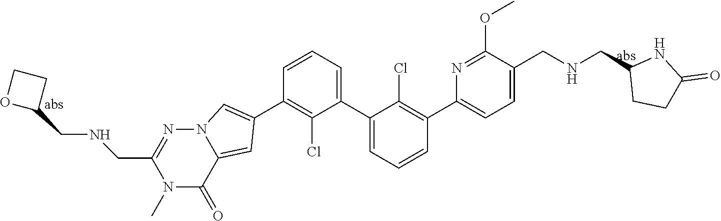

A solution of tert-butyl N-[[6-[2-chloro-3-[2-chloro-3-[3-methyl-2-[[[(2S)-oxetan-2-yl]methylamino]methyl]-4-oxo-pyrrolo[2,1-f][1,2,4]triazin-6-yl]phenyl]phenyl]-2-methoxy-3-pyridyl]methyl]-N-[[(2S)-5-oxopyrrolidin-2-yl]methyl]carbamate (20 mg, 0.02 mmol) in DCM/TFA (2:1) was stirred for 30 min, concentrated and purified by reversed phase chromatography to give 6-[2-chloro-3-[2-chloro-3-[6-methoxy-5-[[[(2S)-5-oxopyrrolidin-2-yl]methylamino]methyl]-2-pyridyl]phenyl]phenyl]-3-methyl-2-[[[(2S)-oxetan-2-yl]methylamino]methyl]pyrrolo[2,1-f][1,2,4]triazin-4-one (16 mg, 91.4%). MS: m/z found 702.2 [M+H]⁺. ¹H NMR (400 MHz, Methanol-d₄): δ 7.85 (d, J=1.9 Hz, 1H), 7.74 (d, J=7.5 Hz, 1H), 7.63 (dt, J=7.7, 1.9 Hz, 2H), 7.46 (dt, J=15.4, 7.6 Hz, 2H), 7.35 (dd, J=7.6, 1.7 Hz, 1H), 7.30-7.23 (m, 3H), 5.00 (qd, J=6.9, 3.9 Hz, 1H), 4.69 (ddd, J=8.6, 7.5, 5.8 Hz, 1H), 4.56 (dt, J=9.2, 5.9 Hz, 1H), 4.03 (s, 3H), 3.99-3.94 (m, 2H), 3.93-3.80 (m, 3H), 3.60 (s, 3H), 3.06 (dd, J=12.9, 6.7 Hz, 1H), 2.95 (dd, J=12.9, 4.0 Hz, 1H), 2.82-2.64 (m, 3H), 2.59-2.47 (m, 1H), 2.42-2.21 (m, 3H), 1.92-1.73 (m, 1H).

### Example 474: 6-[2-Chloro-3-[2-chloro-3-[6-methoxy-5-[[[(2S)-5-oxopyrrolidin-2-yl]methylamino]methyl]-2-pyridyl]phenyl]phenyl]-3-methyl-2-[[[(2S)-tetrahydrofuran-2-yl]methylamino]methyl]pyrrolo[2,1-f][1,2,4]triazin-4-one

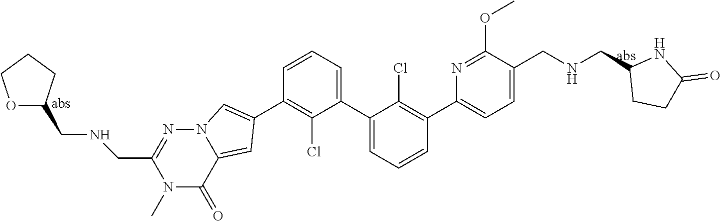

### tert-Butyl N-[[6-[2-chloro-3-[2-chloro-3-[3-methyl-4-oxo-2-[[[(2S)-tetrahydrofuran-2-yl]methylamino]methyl]pyrrolo[2,1-f][1,2,4]triazin-6-yl]phenyl]phenyl]-2-methoxy-3-pyridyl]methyl]-N-[[(2S)-5-oxopyrrolidin-2-yl]methyl]carbamate

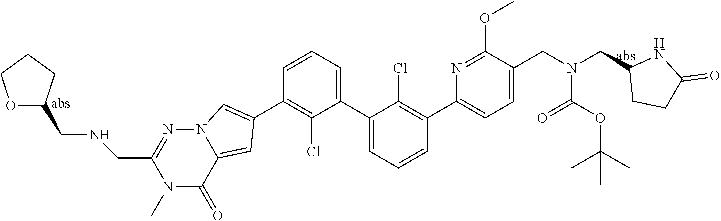

A mixture of tert-butyl N-[[6-[2-chloro-3-[2-chloro-3-(2-formyl-3-methyl-4-oxo-pyrrolo[2,1-f][1,2,4]triazin-6-yl)phenyl]phenyl]-2-methoxy-3-pyridyl]methyl]-N-[[(2S)-5-oxopyrrolidin-2-yl]methyl]carbamate (30 mg, 0.04 mmol), [(2S)-tetrahydrofuran-2-yl]methanamine (8.29 mg, 0.08 mmol) and acetic acid (2.4 uL, 2.5 mg, 0.04 mmol) in MeOH/THF (1:1) was stirred for 1.5 hour at rt. NaBH₃CN (5.15 mg, 0.08 mmol) was added, the mixture was stirred for 30 min, quenched with water and directly purified by reversed phase chromatography to give tert-butyl N-[[6-[2-chloro-3-[2-chloro-3-[3-methyl-4-oxo-2-[[[(2S)-tetrahydrofuran-2-yl]methylamino]methyl]pyrrolo[2,1-f][1,2,4]triazin-6-yl]phenyl]phenyl]-2-methoxy-3-pyridyl]methyl]-N-[[(2S)-5-oxopyrrolidin-2-yl]methyl]carbamate (26 mg, 77.6%). MS: m/z found 816.3 [M+H]⁺.

### 6-[2-Chloro-3-[2-chloro-3-[6-methoxy-5-[[[(2S)-5-oxopyrrolidin-2-yl]methylamino]methyl]-2-pyridyl]phenyl]phenyl]-3-methyl-2-[[[(2S)-tetrahydrofuran-2-yl]methylamino]methyl]pyrrolo[2,1-f][1,2,4]triazin-4-one

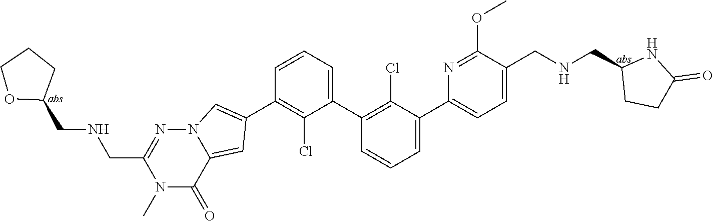 [SMILES: COc1nc(-c2cccc(-c3cccc(-c4cc5c(=O)n(C)c(CNC[C@@H]6CCCO6)nn5c4)c3Cl)c2Cl)ccc1CNC[C@@H]1CCC(=O)N1]

A solution of tert-butyl N-[[6-[2-chloro-3-[2-chloro-3-[3-methyl-4-oxo-2-[[[(2S)-tetrahydrofuran-2-yl]methylamino]methyl]pyrrolo[2,1-f][1,2,4]triazin-6-yl]phenyl]phenyl]-2-methoxy-3-pyridyl]methyl]-N-[[(2S)-5-oxopyrrolidin-2-yl]methyl]carbamate (25 mg, 0.03 mmol) in DCM/TFA (2:1) was stirred for 30 min, concentrated and purified by reversed phase chromatography to give 6-[2-chloro-3-[2-chloro-3-[6-methoxy-5-[[[(2S)-5-oxopyrrolidin-2-yl]methylamino]methyl]-2-pyridyl]phenyl]phenyl]-3-methyl-2-[[[(2S)-tetrahydrofuran-2-yl]methylamino]methyl]pyrrolo[2,1-f][1,2,4]triazin-4-one (21 mg, 96%). MS: m/z found 716.3 [M+H]⁺. ¹H NMR (400 MHz, Methanol-d₄): δ 7.86 (d, J=1.9 Hz, 1H), 7.75 (d, J=7.5 Hz, 1H), 7.63 (dt, J=7.7, 1.9 Hz, 2H), 7.46 (dt, J=15.5, 7.6 Hz, 2H), 7.36 (dd, J=7.6, 1.7 Hz, 1H), 7.30-7.23 (m, 3H), 4.11-4.05 (m, 1H), 4.03 (s, 3H), 3.99 (s, 2H), 3.91-3.84 (m, 4H), 3.76 (q, J=7.3 Hz, 1H), 3.58 (s, 3H), 2.87 (dd, J=12.3, 3.9 Hz, 1H), 2.84-2.79 (m, 1H), 2.79-2.69 (m, 2H), 2.38-2.24 (m, 3H), 2.09-1.98 (m, 1H), 1.97-1.88 (m, 2H), 1.86-1.77 (m, 1H), 1.62 (dt, J=19.1, 7.5 Hz, 1H).

### Example 475: 6-[2-Chloro-3-[2-chloro-3-[6-methoxy-5-[[[(2S)-5-oxopyrrolidin-2-yl]methylamino]methyl]-2-pyridyl]phenyl]phenyl]-2-[[[(2S)-2-methoxypropyl]amino]methyl]-3-methyl-pyrrolo[2,1-f][1,2,4]triazin-4-one

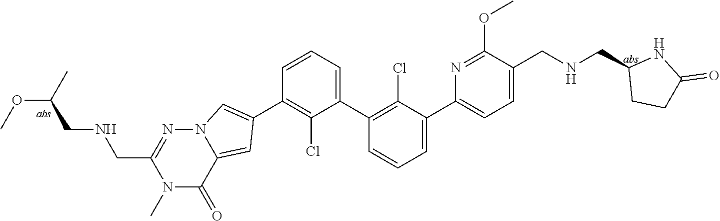 [SMILES: COc1nc(-c2cccc(-c3cccc(-c4cc5c(=O)n(C)c(CNC[C@H](C)OC)nn5c4)c3Cl)c2Cl)ccc1CNC[C@@H]1CCC(=O)N1]

### tert-butyl N-[[6-[2-chloro-3-[2-chloro-3-[2-[[[(2S)-2-methoxypropyl]amino]methyl]-3-methyl-4-oxo-pyrrolo[2,1-f][1,2,4]triazin-6-yl]phenyl]phenyl]-2-methoxy-3-pyridyl]methyl]-N-[[(2S)-5-oxopyrrolidin-2-yl]methyl]carbamate

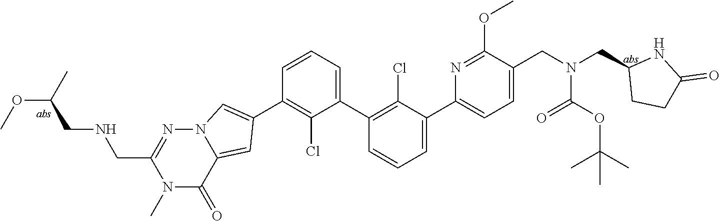 [SMILES: COc1nc(-c2cccc(-c3cccc(-c4cc5c(=O)n(C)c(CNC[C@H](C)OC)nn5c4)c3Cl)c2Cl)ccc1CN(C[C@@H]1CCC(=O)N1)C(=O)OC(C)(C)C]

A mixture of tert-butyl N-[[6-[2-chloro-3-[2-chloro-3-(2-formyl-3-methyl-4-oxo-pyrrolo[2,1-f1][1,2,4]triazin-6-yl)phenyl]phenyl]-2-methoxy-3-pyridyl]methyl]-N-[[(2S)-5-oxopyrrolidin-2-yl]methyl]carbamate (30 mg, 0.04 mmol), (2S)-2-methoxypropan-1-amine (7 mg, 0.08 mmol) and acetic acid (2.4 uL, 2.5 mg, 0.04 mmol) in MeOH/THF (1:1) was stirred for 1.5 hour at rt. NaBH₃CN (5.15 mg, 0.08 mmol) was added, the mixture was stirred for 30 min, quenched with water and directly purified by reversed phase chromatography to give tert-butyl N-[[6-[2-chloro-3-[2-chloro-3-[2-[[[(2S)-2-methoxypropyl]amino]methyl]-3-methyl-4-oxo-pyrrolo[2,1-f][1,2,4]triazin-6-yl]phenyl]phenyl]-2-methoxy-3-pyridyl]methyl]-N-[[(2S)-5-oxopyrrolidin-2-yl]methyl]carbamate (25 mg, 75.8%). MS: m/z found 804.3 [M+H]⁺.

### 6-[2-Chloro-3-[2-chloro-3-[6-methoxy-5-[[[(2S)-5-oxopyrrolidin-2-yl]methylamino]methyl]-2-pyridyl]phenyl]phenyl]-2-[[[(2S)-2-methoxypropyl]amino]methyl]-3-methyl-pyrrolo[2,1-f][1,2,4]triazin-4-one

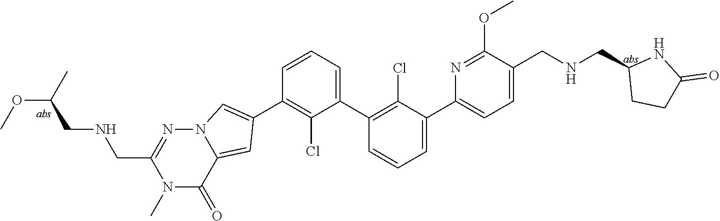 [SMILES: COc1nc(-c2cccc(-c3cccc(-c4cc5c(=O)n(C)c(CNC[C@H](C)OC)nn5c4)c3Cl)c2Cl)ccc1CNC[C@@H]1CCC(=O)N1]

A solution of tert-butyl N-[[6-[2-chloro-3-[2-chloro-3-[2-[[[(2S)-2-methoxypropyl]amino]methyl]-3-methyl-4-oxo-pyrrolo[2,1-f][1,2,4]triazin-6-yl]phenyl]phenyl]-2-methoxy-3-pyridyl]methyl]-N-[[(2S)-5-oxopyrrolidin-2-yl]methyl]carbamate (30 mg, 0.04 mmol) in DCM/TFA (2:1) was stirred for 30 min, concentrated and purified by reversed phase chromatography to give 6-[2-chloro-3-[2-chloro-3-[6-methoxy-5-[[[(2S)-5-oxopyrrolidin-2-yl]methylamino]methyl]-2-pyridyl]phenyl]phenyl]-2-[[[(2S)-2-methoxypropyl]amino]methyl]-3-methyl-pyrrolo[2,1-f][1,2,4]triazin-4-one (25 mg, 95.2%). MS: m/z found 704.3 [M+H]⁺. ¹H NMR (400 MHz, Methanol-d₄): δ 7.87 (d, J=1.9 Hz, 1H), 7.76 (d, J=7.5 Hz, 1H), 7.64 (dt, J=7.6, 1.9 Hz, 2H), 7.47 (dt, J=16.4, 7.7 Hz, 2H), 7.36 (dd, J=7.6, 1.7 Hz, 1H), 7.30-7.24 (m, 3H), 4.04 (s, 3H), 4.01 (s, 2H), 3.97-3.91 (m, 2H), 3.90-3.85 (m, 1H), 3.62-3.54 (m, 4H), 3.39 (s, 3H), 2.88-2.75 (m, 4H), 2.39-2.25 (m, 3H), 1.90-1.77 (m, 1H), 1.18 (d, J=6.3 Hz, 3H).

### Example 489: Methyl (3S)-4-[[6-[2-chloro-3-[2-chloro-3-[6-methoxy-5-[[[(2S)-5-oxopyrrolidin-2-yl]methylamino]methyl]-2-pyridyl]phenyl]phenyl]-3-methyl-4-oxo-pyrrolo[2,1-f][1,2,4]triazin-2-yl]methylamino]-3-hydroxy-butanoate

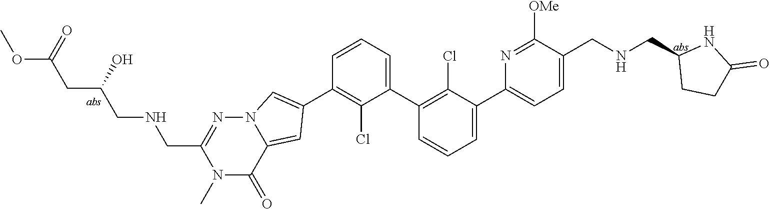 [SMILES: COC(=O)C[C@H](O)CNCc1nn2cc(-c3cccc(-c4cccc(-c5ccc(CNC[C@@H]6CCC(=O)N6)c(OC)n5)c4Cl)c3Cl)cc2c(=O)n1C]

### Methyl (3S)-4-[[6-[3-[3-[5-[[tert-butoxycarbonyl-[[(2S)-5-oxopyrrolidin-2-yl]methyl]amino]methyl]-6-methoxy-2-pyridyl]-2-chloro-phenyl]-2-chloro-phenyl]-3-methyl-4-oxo-pyrrolo[2,1-f][1,2,4]triazin-2-yl]methylamino]-3-hydroxy-butanoate

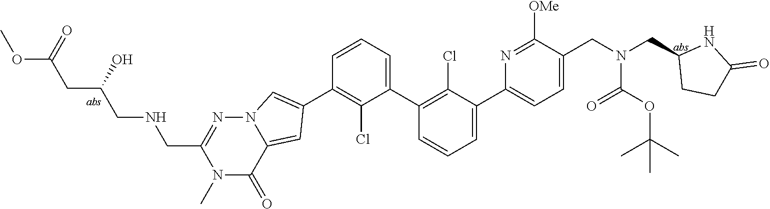 [SMILES: COC(=O)C[C@H](O)CNCc1nn2cc(-c3cccc(-c4cccc(-c5ccc(CN(C[C@@H]6CCC(=O)N6)C(=O)OC(C)(C)C)c(OC)n5)c4Cl)c3Cl)cc2c(=O)n1C]

A mixture of tert-butyl N-[[6-[2-chloro-3-[2-chloro-3-(2-formyl-3-methyl-4-oxo-pyrrolo[2,1-f1][1,2,4]triazin-6-yl)phenyl]phenyl]-2-methoxy-3-pyridyl]methyl]-N-[[(2S)-5-oxopyrrolidin-2-yl]methyl]carbamate (25 mg, 0.03 mmol), methyl (3S)-4-amino-3-hydroxy-butanoate (9 mg, 0.07 mmol) and acetic acid (2 uL, 2 mg, 0.03 mmol) in MeOH/THF (1:1) was stirred for 30 min at rt. NaBH₃CN (4 mg, 0.07 mmol) was added, the mixture was stirred for 20 min, quenched with water and directly purified by reversed phase chromatography to give methyl (3S)-4-[[6-[3-[3-[5-[[tert-butoxycarbonyl-[[(2S)-5-oxopyrrolidin-2-yl]methyl]amino]methyl]-6-methoxy-2-pyridyl]-2-chloro-phenyl]-2-chloro-phenyl]-3-methyl-4-oxo-pyrrolo[2,1-f][1,2,4]triazin-2-yl]methylamino]-3-hydroxy-butanoate (13 mg, 44.8%). MS: m/z found 848.3 [M+H]⁺.

### Methyl (3S)-4-[[6-[2-chloro-3-[2-chloro-3-[6-methoxy-5-[[[(2S)-5-oxopyrrolidin-2-yl]methylamino]methyl]-2-pyridyl]phenyl]phenyl]-3-methyl-4-oxo-pyrrolo[2,1-f][1,2,4]triazin-2-yl]methylamino]-3-hydroxy-butanoate

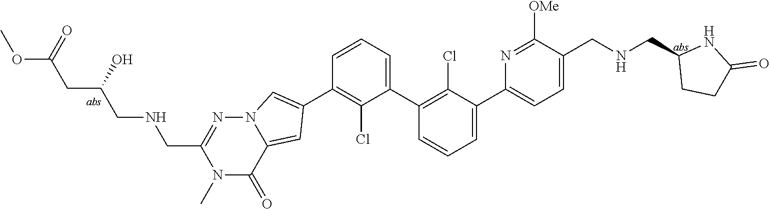 [SMILES: COC(=O)C[C@H](O)CNCc1nn2cc(-c3cccc(-c4cccc(-c5ccc(CNC[C@@H]6CCC(=O)N6)c(OC)n5)c4Cl)c3Cl)cc2c(=O)n1C]

A solution of methyl (3 S)-4-[[6-[3-[3-[5-[[tert-butoxycarbonyl-[[(2S)-5-oxopyrrolidin-2-yl]methyl]amino]methyl]-6-methoxy-2-pyridyl]-2-chloro-phenyl]-2-chloro-phenyl]-3-methyl-4-oxo-pyrrolo[2,1-f][1,2,4]triazin-2-yl]methylamino]-3-hydroxy-butanoate (10 mg, 0.01 mmol) in DCM/TFA (2:1) was stirred for 30 min, concentrated and purified by reversed phase chromatography to give methyl (3S)-4-[[6-[2-chloro-3-[2-chloro-3-[6-methoxy-5-[[[(2S)-5-oxopyrrolidin-2-yl]methylamino]methyl]-2-pyridyl]phenyl]phenyl]-3-methyl-4-oxo-pyrrolo[2,1-f][1,2,4]triazin-2-yl]methylamino]-3-hydroxy-butanoate (8.5 mg, 96.4%). MS: m/z found 748.2 [M+H]⁺. ¹H NMR (400 MHz, Methanol-d₄): δ 7.86 (d, J=1.9 Hz, 1H), 7.73 (d, J=7.4 Hz, 1H), 7.64 (t, J=2.0 Hz, 1H), 7.62 (t, J=1.9 Hz, 1H), 7.51-7.42 (m, 2H), 7.35 (dd, J=7.6, 1.7 Hz, 1H), 7.30-7.23 (m, 3H), 4.16 (dq, J=8.6, 4.7, 3.9 Hz, 1H), 4.03 (s, 3H), 4.00-3.89 (m, 2H), 3.88-3.78 (m, 3H), 3.65 (s, 3H), 3.60 (s, 3H), 2.85-2.73 (m, 2H), 2.73-2.64 (m, 2H), 2.59 (dd, J=15.4, 4.8 Hz, 1H), 2.46 (dd, J=15.3, 8.1 Hz, 1H), 2.38-2.25 (m, 3H), 1.88-1.76 (m, 1H).

### Example 493: (3S)-4-[[6-[2-Chloro-3-[2-chloro-3-[6-methoxy-5-[[[(2S)-5-oxopyrrolidin-2-yl]methylamino]methyl]-2-pyridyl]phenyl]phenyl]-3-methyl-4-oxo-pyrrolo[2,1-f][1,2,4]triazin-2-yl]methylamino]-3-hydroxy-butanoic acid

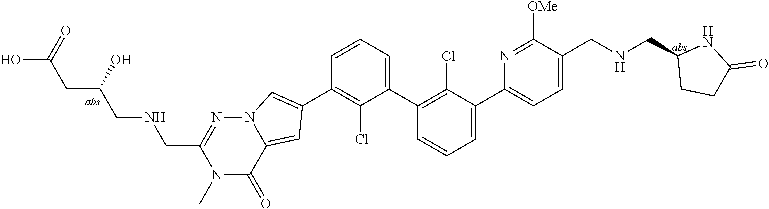 [SMILES: COc1nc(-c2cccc(-c3cccc(-c4cc5c(=O)n(C)c(CNC[C@@H](O)CC(=O)O)nn5c4)c3Cl)c2Cl)ccc1CNC[C@@H]1CCC(=O)N1]

### (3S)-4-[[6-[3-[3-[5-[[tert-Butoxycarbonyl-[[(2S)-5-oxopyrrolidin-2-yl]methyl]amino]methyl]-6-methoxy-2-pyridyl]-2-chloro-phenyl]-2-chloro-phenyl]-3-methyl-4-oxo-pyrrolo[2,1-f][1,2,4]triazin-2-yl]methylamino]-3-hydroxy-butanoic acid

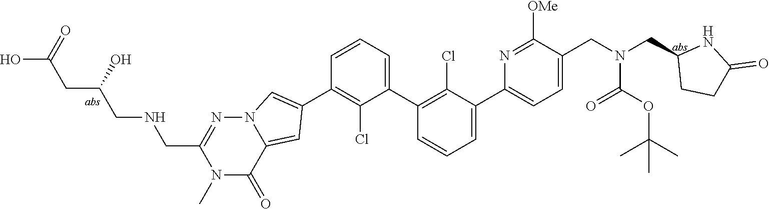 [SMILES: COc1nc(-c2cccc(-c3cccc(-c4cc5c(=O)n(C)c(CNC[C@@H](O)CC(=O)O)nn5c4)c3Cl)c2Cl)ccc1CN(C[C@@H]1CCC(=O)N1)C(=O)OC(C)(C)C]

A mixture of tert-butyl N-[[6-[2-chloro-3-[2-chloro-3-(2-formyl-3-methyl-4-oxo-pyrrolo[2,1-f1][1,2,4]triazin-6-yl)phenyl]phenyl]-2-methoxy-3-pyridyl]methyl]-N-[[(2S)-5-oxopyrrolidin-2-yl]methyl]carbamate (10 mg, 0.01 mmol), (3S)-4-amino-3-hydroxy-butanoic acid (3.26 mg, 0.03 mmol) and acetic acid (1 uL, 1 mg, 0.01 mmol) in MeOH/THF (1:1) was stirred for overnight at rt. NaBH₃CN (2 mg, 0.03 mmol) was added, the mixture was stirred for 2 h, quenched with water and directly purified by reversed phase chromatography to give (3S)-4-[[6-[3-[3-[5-[[tert-butoxycarbonyl-[[(2S)-5-oxopyrrolidin-2-yl]methyl]amino]methyl]-6-methoxy-2-pyridyl]-2-chloro-phenyl]-2-chloro-phenyl]-3-methyl-4-oxo-pyrrolo[2,1-f][1,2,4]triazin-2-yl]methylamino]-3-hydroxy-butanoic acid (6 mg, 52.6%). MS: m/z found 834.3 [M+H]⁺.

### (3S)-4-[[6-[2-Chloro-3-[2-chloro-3-[6-methoxy-5-[[[(2S)-5-oxopyrrolidin-2-yl]methylamino]methyl]-2-pyridyl]phenyl]phenyl]-3-methyl-4-oxo-pyrrolo[2,1-f][1,2,4]triazin-2-yl]methylamino]-3-hydroxy-butanoic acid

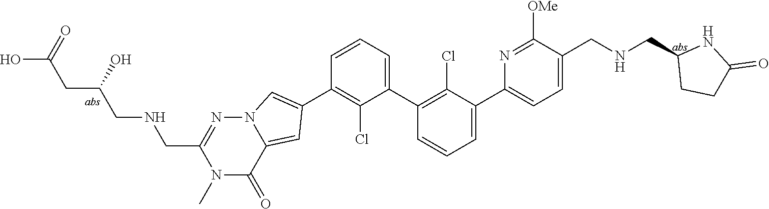 [SMILES: COc1nc(-c2cccc(-c3cccc(-c4cc5c(=O)n(C)c(CNC[C@@H](O)CC(=O)O)nn5c4)c3Cl)c2Cl)ccc1CNC[C@@H]1CCC(=O)N1]

A solution of (3S)-4-[[6-[3-[3-[5-[[tert-butoxycarbonyl-[[(2S)-5-oxopyrrolidin-2-yl]methyl]amino]methyl]-6-methoxy-2-pyridyl]-2-chloro-phenyl]-2-chloro-phenyl]-3-methyl-4-oxo-pyrrolo[2,1-f][1,2,4]triazin-2-yl]methylamino]-3-hydroxy-butanoic acid (5 mg, 0.01 mmol) in DCM/TFA (2:1) was stirred for 30 min, concentrated and purified by reversed phase chromatography to give (3S)-4-[[6-[2-chloro-3-[2-chloro-3-[6-methoxy-5-[[[(2S)-5-oxopyrrolidin-2-yl]methylamino]methyl]-2-pyridyl]phenyl]phenyl]-3-methyl-4-oxo-pyrrolo[2,1-f][1,2,4]triazin-2-yl]methylamino]-3-hydroxy-butanoic acid (4 mg, 90.9%). MS: m/z found 734.2 [M+H]⁺. ¹H NMR (400 MHz, Methanol-d₄): δ 7.90 (d, J=1.9 Hz, 1H), 7.74 (d, J=7.5 Hz, 1H), 7.64 (dd, J=3.4, 1.7 Hz, 1H), 7.63 (dd, J=3.3, 1.7 Hz, 1H), 7.51-7.42 (m, 2H), 7.36 (dd, J=7.6, 1.7 Hz, 1H), 7.30-7.24 (m, 3H), 4.20-4.12 (m, 1H), 4.11-4.06 (m, 2H), 4.03 (s, 3H), 3.92-3.80 (m, 3H), 3.57 (s, 3H), 2.98 (dd, J=12.3, 3.9 Hz, 1H), 2.87 (dd, J=12.2, 7.7 Hz, 1H), 2.80-2.66 (m, 2H), 2.43 (dd, J=6.2, 3.6 Hz, 2H), 2.38-2.25 (m, 3H), 1.87-1.77 (m, 1H).

### Example 501: (3S)-1-[[6-[2-Chloro-3-[2-chloro-3-[6-methoxy-5-[[[(2S)-5-oxopyrrolidin-2-yl]methylamino]methyl]-2-pyridyl]phenyl]phenyl]-3-methyl-4-oxo-pyrrolo[2,1-f][1,2,4]triazin-2-yl]methyl]pyrrolidine-3-carboxylic acid

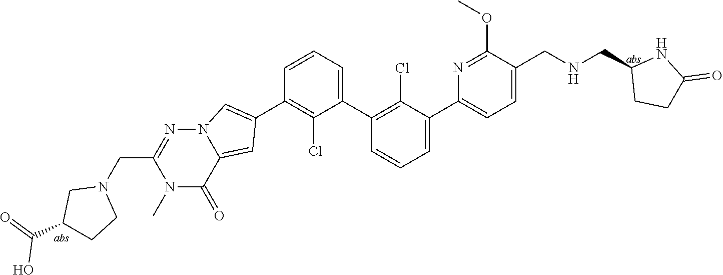 [SMILES: COc1nc(-c2cccc(-c3cccc(-c4cc5c(=O)n(C)c(CN6CC[C@H](C(=O)O)C6)nn5c4)c3Cl)c2Cl)ccc1CNC[C@@H]1CCC(=O)N1]

### (3S)-1-116-13-13-15-1[tert-Butoxycarbonyl-1[(2S)-5-oxopyrrolidin-2-yl]methyl]amino]methyl]-6-methoxy-2-pyridyl]-2-chloro-phenyl]-2-chloro-phenyl]-3-methyl-4-oxo-pyrrolo[2,1-f][1,2,4]triazin-2-yl]methyl]pyrrolidine-3-carboxylic acid

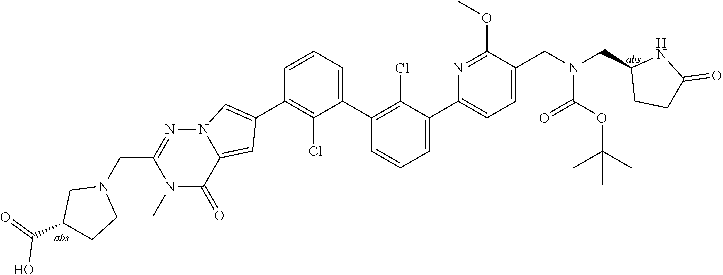 [SMILES: COc1nc(-c2cccc(-c3cccc(-c4cc5c(=O)n(C)c(CN6CC[C@H](C(=O)O)C6)nn5c4)c3Cl)c2Cl)ccc1CN(C[C@@H]1CCC(=O)N1)C(=O)OC(C)(C)C]

A mixture of tert-butyl N-[[6-[2-chloro-3-[2-chloro-3-(2-formyl-3-methyl-4-oxo-pyrrolo[2,1-f][1,2,4]triazin-6-yl)phenyl]phenyl]-2-methoxy-3-pyridyl]methyl]-N-[[(2S)-5-oxopyrrolidin-2-yl]methyl]carbamate (13 mg, 0.02 mmol), (3S)-pyrrolidine-3-carboxylic acid (4.1 mg, 0.04 mmol) and acetic acid (1.0 uL, 1.1 mg, 0.02 mmol) in MeOH/THF (1:1) was stirred for overnight at rt. NaBH₃CN (2.23 mg, 0.04 mmol) was added, the mixture was stirred for 2 h, quenched with water and directly purified by reversed phase chromatography to give (3S)-1-[[6-[3-[3-[5-[[tert-butoxycarbonyl-[[(2S)-5-oxopyrrolidin-2-yl]methyl]amino]methyl]-6-methoxy-2-pyridyl]-2-chloro-phenyl]-2-chloro-phenyl]-3-methyl-4-oxo-pyrrolo[2,1-f][1,2,4]triazin-2-yl]methyl]pyrrolidine-3-carboxylic acid (7 mg, 47.4%). MS: m/z found 830.3 [M+H]⁺.

### (3S)-1-[[6-[2-Chloro-3-[2-chloro-3-[6-methoxy-5-[[[(2S)-5-oxopyrrolidin-2-yl]methylamino]methyl]-2-pyridyl]phenyl]phenyl]-3-methyl-4-oxo-pyrrolo[2,1-f][1,2,4]triazin-2-yl]methyl]pyrrolidine-3-carboxylic acid

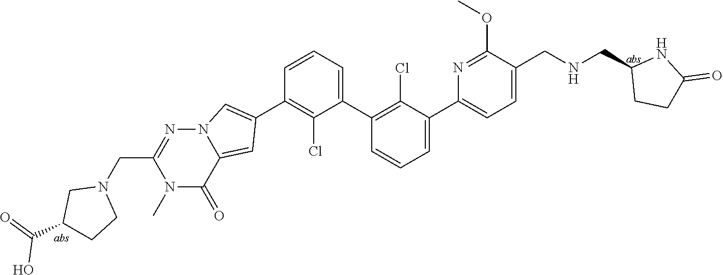 [SMILES: COc1nc(-c2cccc(-c3cccc(-c4cc5c(=O)n(C)c(CN6CC[C@H](C(=O)O)C6)nn5c4)c3Cl)c2Cl)ccc1CNC[C@@H]1CCC(=O)N1]

A solution of (3S)-1-[[6-[3-[3-[5-[[tert-butoxycarbonyl-[[(2S)-5-oxopyrrolidin-2-yl]methyl]amino]methyl]-6-methoxy-2-pyridyl]-2-chloro-phenyl]-2-chloro-phenyl]-3-methyl-4-oxo-pyrrolo[2,1-f][1,2,4]triazin-2-yl]methyl]pyrrolidine-3-carboxylic acid (5 mg, 0.01 mmol) in DCM/TFA (2:1) was stirred for 30 min, concentrated and purified by reversed phase chromatography to give (3S)-1-[[6-[2-chloro-3-[2-chloro-3-[6-methoxy-5-[[[(2S)-5-oxopyrrolidin-2-yl]methylamino]methyl]-2-pyridyl]phenyl]phenyl]-3-methyl-4-oxo-pyrrolo[2,1-f][1,2,4]triazin-2-yl]methyl]pyrrolidine-3-carboxylic acid (4.3 mg, 97.8%). MS: m/z found 730.2 [M+H]⁺. ¹H NMR (400 MHz, Methanol-d₄): δ 7.86 (d, J=1.9 Hz, 1H), 7.77 (d, J=7.4 Hz, 1H), 7.64 (dt, J=7.7, 1.9 Hz, 2H), 7.49 (t, J=7.7 Hz, 1H), 7.45 (t, J=7.7 Hz, 1H), 7.36 (dd, J=7.6, 1.7 Hz, 1H), 7.30-7.25 (m, 3H), 4.04 (s, 3H), 3.98-3.93 (m, 2H), 3.93-3.85 (m, 3H), 3.64 (s, 3H), 3.10-2.99 (m, 3H), 2.92-2.77 (m, 4H), 2.39-2.26 (m, 3H), 2.20-2.09 (m, 2H), 1.89-1.79 (m, 1H).

### Example 502: 1-[[6-[2-Chloro-3-[2-chloro-3-[6-methoxy-5-[[[(2S)-5-oxopyrrolidin-2-yl]methylamino]methyl]-2-pyridyl]phenyl]phenyl]-3-methyl-4-oxo-pyrrolo[2,1-f][1,2,4]triazin-2-yl]methylamino]cyclopropanecarboxylic acid

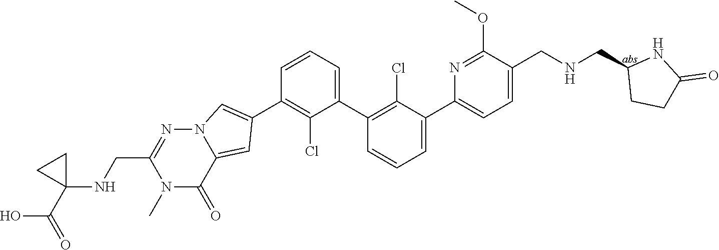 [SMILES: COc1nc(-c2cccc(-c3cccc(-c4cc5c(=O)n(C)c(CNC6(C(=O)O)CC6)nn5c4)c3Cl)c2Cl)ccc1CNC[C@@H]1CCC(=O)N1]

### 1-11[6-13-13-15-[[tert-Butoxycarbonyl-1[(2S)-5-oxopyrrolidin-2-yl]1methyl]amino]methyl]-6-methoxy-2-pyridyl]-2-chloro-phenyl]-2-chloro-phenyl-3-methyl-4-oxo-pyrrolo[2,1-f][1,2,4]triazin-2-ylmethylamino]cyclopropanecarboxylic acid

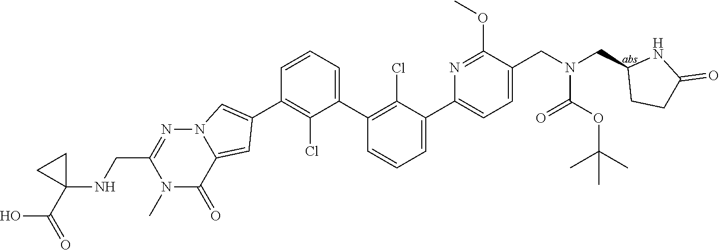 [SMILES: COc1nc(-c2cccc(-c3cccc(-c4cc5c(=O)n(C)c(CNC6(C(=O)O)CC6)nn5c4)c3Cl)c2Cl)ccc1CN(C[C@@H]1CCC(=O)N1)C(=O)OC(C)(C)C]

A mixture of tert-butyl N-[[6-[2-chloro-3-(2-formyl-3methyl-4-oxo-pyrrolo[2,1-f][1,2,4]triazin-6-yl)phenyl]phenyl]-2-methoxy-3-pyridyl]methyl]-N-[[(2S)-5-oxopyrrolidin-2-yl]methyl]carbamate (15 mg, 0.02 mmol), 1-aminocyclopropanecarboxylic acid (4.15 mg, 0.04 mmol) and acetic acid (1.2 uL, 1.2 mg, 0.02 mmol) in MeOH/THF (1:1) was stirred for overnight at rt. NaBH₃ CN (2.6 mg, 0.04 mmol) was added, the mixture was stirred for 2 h, quenched with water and directly purified by reversed phase chromatography to give 1[[6-[3-[3-[5-[[tert-butoxycarbonyl-[[(2S)-5-oxopyrrolidin-2-yl]methyl]amino]methyl]-6-methoxy-2-pyridyl]-2-chloro-phenyl]-2-chloro-phenyl]-3-methyl-4-oxo-pyrrolo[2,1-f][1,2,4]triazin-2-yl]methylamino]cyclopropanecarboxylic acid (12 mg, 71.7%). MS: m/z found 816.3 [M+H]⁺.

### 1-[[6-[2-chloro-3-[2-Chloro-3-[6-methoxy-5-[[[(2S)-5-oxopyrrolidin-2-yl]methylamino]methyl]-2-pyridyl]phenyl]phenyl]-3-methyl-4-oxo-pyrrolo[2,1-f][1,2,4]triazin-2-yl]methylamino]cyclopropanecarboxylic acid

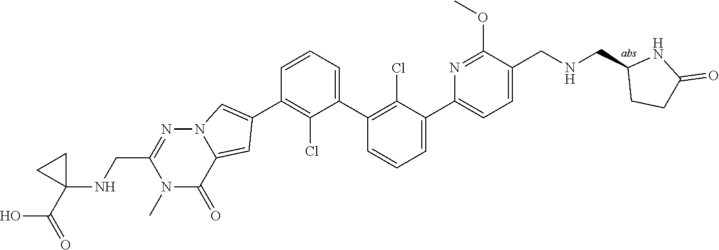 [SMILES: COc1nc(-c2cccc(-c3cccc(-c4cc5c(=O)n(C)c(CNC6(C(=O)O)CC6)nn5c4)c3Cl)c2Cl)ccc1CNC[C@@H]1CCC(=O)N1]

A solution of 1-[[6-[3-[3-[5-[[tert-butoxycarbonyl-[[(2S)-5-oxopyrrolidin-2-yl]methyl]amino]methyl]-6-methoxy-2-pyridyl]-2-chloro-phenyl]-2-chloro-phenyl]-3-methyl-4-oxo-pyrrolo[2,1-f][1,2,4]triazin-2-yl]methylamino]cyclopropanecarboxylic acid (10 mg, 0.01 mmol) in DCM/TFA (2:1) was stirred for 30 min, concentrated and purified by reversed phase chromatography to give 1-[[6-[2-chloro-3-[2-chloro-3-[6-methoxy-5-[[[(2S)-5-oxopyrrolidin-2-yl]methylamino]methyl]-2-pyridyl]phenyl]phenyl]-3-methyl-4-oxo-pyrrolo[2,1-f][1,2,4]triazin-2-yl]methylamino]cyclopropanecarboxylic acid (8 mg, 91.2 0%). MS: m/z found 716.2 [M+H]⁺. ¹H NMR (400 MHz, Methanol-d₄): δ7.84 (d, J=1.9 Hz, 1H), 7.79 (d, J=7.5 Hz, 1H), 7.65-7.60 (m, 2H), 7.48 (t, J=7.6 Hz, 1H), 7.44 (t, J=7.7 Hz, 1H), 7.36 (dd, J=7.6, 1.7 Hz, 1H), 7.30 (d, J=7.5 Hz, 1H), 7.28-7.24 (m, 2H), 4.10 (s, 2H), 4.05 (s, 3H), 4.03-4.00 (m, 2H), 3.96-3.89 (m, 1H), 3.60 (s, 3H), 2.97-2.86 (in, 2H), 2.40-2.27 (m, 3H), 1.84 (ddd, J=11.3, 9.1, 5.6 Hz, 1H), 1.29 (q, J=4.1 Hz, 2H), 1.02 (q, J=4.1 Hz, 2H).

### Example 330: N-((6-(2,2′-Dichloro-3′-(6-methoxy-5-(((((S)-5-oxopyrrolidin-2-yl)methyl)amino)methyl)pyridin-2-yl)-[1,1′-biphenyl]-3-yl)-2-methoxypyridin-3-yl)methyl)-N—(((S)-5-oxopyrrolidin-2-yl)methyl)formamide

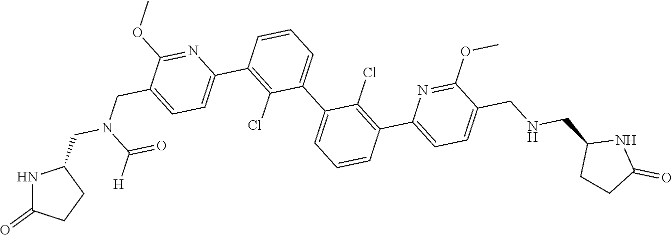 [SMILES: COc1nc(-c2cccc(-c3cccc(-c4ccc(CN(C=O)C[C@@H]5CCC(=O)N5)c(OC)n4)c3Cl)c2Cl)ccc1CNC[C@@H]1CCC(=O)N1]

### 6,6′-(2,2′-Dichloro-[1,1′-biphenyl]-3,3′-diyl)bis(2-methoxynicotinaldehyde)

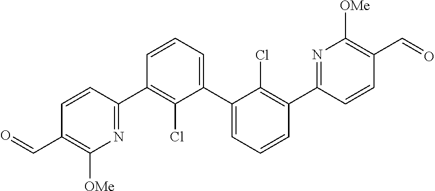 [SMILES: COc1nc(-c2cccc(-c3cccc(-c4ccc(C=O)c(OC)n4)c3Cl)c2Cl)ccc1C=O]

To a mixture of 6-(3-bromo-2-chlorophenyl)-2-methoxynicotinaldehyde (2 g, 6.12 mmol) and 6-(2-chloro-3-(4,4,5,5-tetramethyl-1,3,2-dioxaborolan-2-yl)phenyl)-2-methoxynicotinaldehyde (2.3 g, 6.12 mmol) in THE (40 mL) and H₂O (10 mL) were added 1,1′-bis(diphenylphosphino)ferrocene-palladium(ii) dichloride dichloromethane complex (0.5 g, 0.6 mmol) and cesium carbonate (6 g, 18.4 mmol). The mixture was stirred at 95° C. for 3 h. The reaction mixture was treated with water (10 mL), and then extracted with EtOAc (2×10 mL). The combined organic layers were washed with saturated aqueous brine solution (10 mL), dried over anhydrous sodium sulfate, filtered, and concentrated under reduced pressure to give a residue. The residue was triturated with PE/EtOAc/THF mixture (2:1:1, 40 mL) to afford 6,6′-(2,2′-dichloro-[1,1′-biphenyl]-3,3′-diyl)bis(2-methoxynicotinaldehyde) (2 g, 66% yield) as a brown solid. ¹H NMR (400 MHz, CDCl₃): δ 10.42 (s, 2H), 8.19 (d, J=7.6 Hz, 2H), 7.68 (d, J=7.6 Hz, 2H), 7.49-7.40 (m, 6H), 4.13 (s, 6H).

### N-((6-(2,2′-Dichloro-3′-(6-methoxy-5-(((((S)-5-oxopyrrolidin-2-yl)methyl)amino)methyl)-pyridin-2-yl)-[1,1′-biphenyl]-3-yl)-2-methoxypyridin-3-yl)methyl)-N—(((S)-5-oxopyrrolidin-2-yl)methyl)formamide

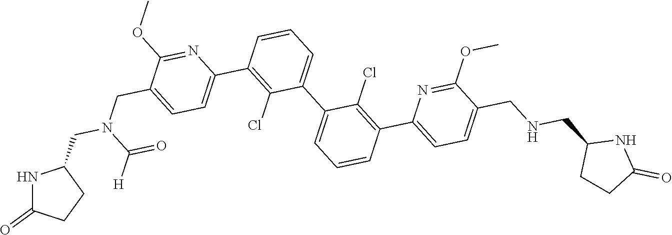 [SMILES: COc1nc(-c2cccc(-c3cccc(-c4ccc(CN(C=O)C[C@@H]5CCC(=O)N5)c(OC)n4)c3Cl)c2Cl)ccc1CNC[C@@H]1CCC(=O)N1]

To a mixture of 6,6′-(2,2′-dichloro-[1,1′-biphenyl]-3,3′-diyl)bis(2-methoxynicotinaldehyde) (2 g, 4 mmol) and (5S)-5-(aminomethyl)pyrrolidin-2-one hydrochloride (3 g, 20.3 mmol) in DCM (10 mL) was added sodium acetate (1.66 g, 20.3 mmol) and 4A molecular sieve (0.02 g). The mixture was stirred at r.t. for 1 h. Sodium triacetoxyborohydride (4.3 g, 20.3 mmol) was added to the mixture and the solution was stirred for 1 h. Reaction was filtered and concentrated under reduced pressure to give a residue. The residue was purified by reverse phase HPLC (with formic acid modifier). The combined product fractions were concentrated under reduced pressure at 45° C. to remove excess water. The aqueous was frozen and lyophilized to afford (5S)-5-[[[6-[2-chloro-3-[2-chloro-3-[6-methoxy-5-[[[(2S)-5-oxopyrrolidin-2-yl]methylamino]methyl]-2-pyridyl]phenyl]phenyl]-2-methoxy-3-pyridyl]methylamino]methyl]pyrrolidin-2-one (1.15 g, m/z: 711) as a white solid. The sample was further purified by reverse phase HPLC to isolate the formyl adduct N-((6-(2,2′-dichloro-3′-(6-methoxy-5-(((((S)-5-oxopyrrolidin-2-yl)methyl)amino)methyl)-pyridin-2-yl)-[1,1′-biphenyl]-3-yl)-2-methoxypyridin-3-yl)methyl)-N—(((S)-5-oxopyrrolidin-2-yl)methyl)formamide (9.6 mg) as a white solid. MS: m/z found 718 [M+H]. ¹H NMR (400 MHz, Chloroform-d) δ 8.29 (dd, J=55.2, 2.7 Hz, 1H), 7.68-7.63 (m, 2H), 7.58 (dd, J=7.1, 3.1 Hz, 1H), 7.43 (dtd, J=6.8, 4.6, 4.2, 2.3 Hz, 2H), 7.34 (ddt, J=15.0, 7.5, 2.7 Hz, 3H), 7.29-7.25 (m, 2H), 6.47 (d, J=6.0 Hz, 1H), 6.30 (s, 1H), 4.79-4.31 (m, 2H), 4.02 (t, J=2.5 Hz, 6H), 3.96-3.71 (m, 5H), 3.40-3.19 (m, 2H), 2.78 (dt, J=12.2, 3.5 Hz, 1H), 2.64-2.50 (m, 1H), 2.47-2.10 (m, 7H).

### Example 494: (S)-1-((6-(2,2′-Dichloro-3′-(6-methoxy-5-(((((S)-5-oxopyrrolidin-2-yl)methyl)amino)-methyl)pyridin-2-yl)-[1,1′-biphenyl]-3-yl)-2-methoxypyridin-3-yl)methyl)piperidine-2-carboxylic acid

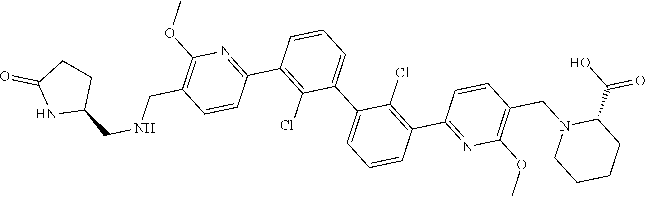 [SMILES: COc1nc(-c2cccc(-c3cccc(-c4ccc(CN5CCCC[C@H]5C(=O)O)c(OC)n4)c3Cl)c2Cl)ccc1CNC[C@@H]1CCC(=O)N1]

### tert-Butyl (S)-((6-(2,2′-dichloro-3′-(5-formyl-6-methoxypyridin-2-yl)-[1,1′-biphenyl]-3-yl)-2-methoxypyridin-3-yl)methyl)((5-oxopyrrolidin-2-yl)methyl)carbamate

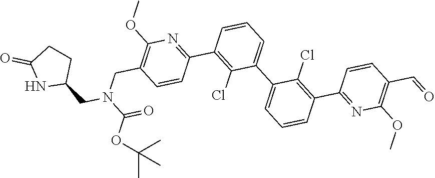 [SMILES: COc1nc(-c2cccc(-c3cccc(-c4ccc(CN(C[C@@H]5CCC(=O)N5)C(=O)OC(C)(C)C)c(OC)n4)c3Cl)c2Cl)ccc1C=O]

Pd(PPh₃)₄ (34 mg, 0.03 mmol), potassium carbonate (61 mg, 0.44 mmol), tert-butyl N-[[6-[2-chloro-3-[2-chloro-3-(4,4,5,5-tetramethyl-1,3,2-dioxaborolan-2-yl)phenyl]phenyl]-2-methoxy-3-pyridyl]methyl]-N-[[(2S)-5-oxopyrrolidin-2-yl]methyl]carbamate (100 mg, 0.15 mmol), and 6-chloro-2-methoxy-pyridine-3-carbaldehyde (33 mg, 0.19 mmol) were suspended in dioxane/water (4:1, 3 mL), then the solution was heated at 105° C. for 20 minutes. Reaction was cooled to rt, diluted with 5 mL water, and extracted with EtOAc (3×3 mL). The combined organics were concentrated under reduced pressure and crude sample was purified by normal phase SiO₂ chromatography (0-4% MeOH/DCM) to afford tert-butyl N-[[6-[2-chloro-3-[2-chloro-3-(4,4,5,5-tetramethyl-1,3,2-dioxaborolan-2-yl)phenyl]phenyl]-2-methoxy-3-pyridyl]methyl]-N-[[(2S)-5-oxopyrrolidin-2-yl]methyl]carbamate (100 mg, 0.15 mmol). MS: m/z found 691.3 [M+H]⁺.

### (S)-1-((6-(3′-(5-(((tert-Butoxycarbonyl)(((S)-5-oxopyrrolidin-2-yl)methyl)amino)methyl)-6-methoxypyridin-2-yl)-2,2′-dichloro-[1,1′-biphenyl]-3-yl)-2-methoxypyridin-3-yl)methyl)piperidine-2-carboxylic acid

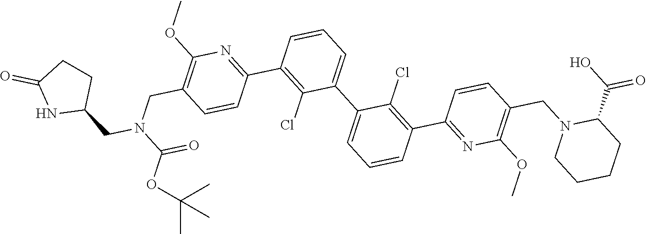 [SMILES: COc1nc(-c2cccc(-c3cccc(-c4ccc(CN5CCCC[C@H]5C(=O)O)c(OC)n4)c3Cl)c2Cl)ccc1CN(C[C@@H]1CCC(=O)N1)C(=O)OC(C)(C)C]

tert-Butyl N-[[6-[2-chloro-3-[2-chloro-3-(5-formyl-6-methoxy-2-pyridyl)phenyl]phenyl]-2-methoxy-3-pyridyl]methyl]-N-[[(2S)-5-oxopyrrolidin-2-yl]methyl]carbamate (30 mg, 0.03 mmol), (2S)-piperidine-2-carboxylic acid (8.40 mg, 0.07 mmol), and acetic acid (2 mg, 0.03 mmol) were dissolved in MeOH/THF (1:1, 1 mL), then the solution was stirred at 25° C. for 12 hours. NaBH₃CN (4.1 mg, 0.07 mmol) was added and the resulting mixture was stirred at 25° C. for 1 hour. Reaction was subsequently diluted with water (2 mL) and extracted with EtOAc (3×2 mL). The combined organics were further washed with brine, dried over sodium sulfate, filtered, and concentrated under reduced pressure. Crude sample was purified by reverse phase HPLC to afford (2S)-1-[[6-[3-[3-[5-[[tert-butoxycarbonyl-[[(2S)-5-oxopyrrolidin-2-yl]-methyl]-amino]-methyl]-6-methoxy-2-pyridyl]-2-chloro-phenyl]-2-chloro-phenyl]-2-methoxy-3-pyridyl]methyl]piperidine-2-carboxylic acid (6 mg, 22.9% yield) as a white solid. MS: m/z found 804.4, 806.3 [M+H]⁺. ¹H NMR (400 MHz, Methanol-d₄) δ 7.97 (d, J=7.6 Hz, 1H), 7.65 (ddd, J=11.0, 7.6, 1.7 Hz, 2H), 7.55-7.46 (m, 3H), 7.43-7.34 (m, 3H), 7.25 (d, J=7.5 Hz, 1H), 4.64-4.34 (m, 5H), 4.06 (s, 3H), 4.00 (s, 5H), 3.54 (s, 1H), 3.42 (s, 3H), 3.05 (s, 1H), 2.42-2.21 (m, 4H), 1.81 (d, J=21.0 Hz, 6H), 1.45 (d, J=42.8 Hz, 9H).

### (S)-1-((6-(2,2′-Dichloro-3′-(6-methoxy-5-(((((S)-5-oxopyrrolidin-2-yl)methyl)amino)-methyl)pyridin-2-yl)-[1,1′-biphenyl]-3-yl)-2-methoxypyridin-3-yl)methyl)piperidine-2-carboxylic acid

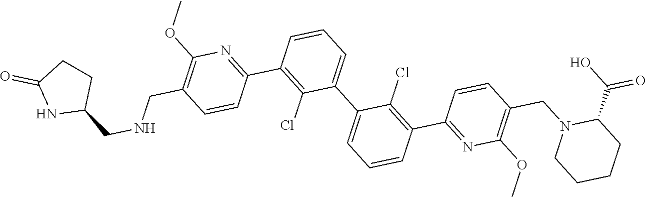 [SMILES: COc1nc(-c2cccc(-c3cccc(-c4ccc(CN5CCCC[C@H]5C(=O)O)c(OC)n4)c3Cl)c2Cl)ccc1CNC[C@@H]1CCC(=O)N1]

(2S)-1-[[6-[3-[3-[5-[[tert-butoxycarbonyl-[[(2S)-5-oxopyrrolidin-2-yl]methyl]amino]methyl]-6-methoxy-2-pyridyl]-2-chloro-phenyl]-2-chloro-phenyl]-2-methoxy-3-pyridyl]methyl]piperidine-2-carboxylic acid (6.00 mg, 0.01 mmol) was dissolved in 1 mL DCM and 4M HCl solution in dioxane (28 μL, 4 mg, 0.11 mmol) was added. Reaction was stirred at rt for 20 minutes and solvent was removed under reduced pressure. Crude sample was purified by reverse phase HPLC to afford (2S)-1-[[6-[2-chloro-3-[2-chloro-3-[6-methoxy-5-[[[(2S)-5-oxopyrrolidin-2-yl]methylamino]methyl]-2-pyridyl]phenyl]phenyl]-2-methoxy-3-pyridyl]methyl]piperidine-2-carboxylic acid (4 mg, 75%) as a white solid (formic acid salt). MS: m/z found 704.3, 706.3 [M+H]⁺. ¹H NMR formic acid salt (400 MHz, Methanol-d₄): δ 8.44 (s, 1H), 7.97 (d, J=7.6 Hz, 1H), 7.77 (d, J=7.6 Hz, 1H), 7.65 (ddd, J=11.0, 7.7, 1.8 Hz, 2H), 7.50 (td, J=7.6, 5.6 Hz, 2H), 7.39 (td, J=7.5, 1.9 Hz, 3H), 7.28 (d, J=7.5 Hz, 1H), 4.51 (d, J=13.1 Hz, 1H), 4.39 (d, J=13.4 Hz, 1H), 4.06 (s, 3H), 4.04 (s, 3H), 3.97 (d, J=3.5 Hz, 2H), 3.92 (d, J=15.4 Hz, 1H), 3.53 (s, 1H), 3.41 (s, 1H), 3.07 (d, J=12.5 Hz, 1H), 2.92-2.77 (m, 2H), 2.32 (qd, J=15.9, 14.6, 6.7 Hz, 4H), 1.93-1.66 (m, 5H), 1.57 (s, 1H).

### Example 508: (S)-6-(2,2′-Dichloro-3′-(4-fluoro-6-methoxy-5-((((5-oxopyrrolidin-2-yl)methyl)amino)methyl)pyridin-2-yl)-[1,1′-biphenyl]-3-yl)-2-(((3-fluoropropyl)amino)methyl)-3-methylpyrrolo[2,1-f][1,2,4]triazin-4(3H)-one

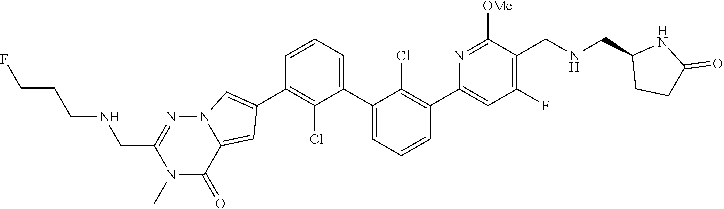 [SMILES: COc1nc(-c2cccc(-c3cccc(-c4cc5c(=O)n(C)c(CNCCCF)nn5c4)c3Cl)c2Cl)cc(F)c1CNC[C@@H]1CCC(=O)N1]

### (S)-tert-Butyl ((6-(2,2′-dichloro-3′-(2-(((3-fluoropropyl)amino)methyl)-3-methyl-4-oxo-3,4-dihydropyrrolo[2,1-f][1,2,4]triazin-6-yl)-[1,1′-biphenyl]-3-yl)-4-fluoro-2-methoxypyridin-3-yl)methyl)((5-oxopyrrolidin-2-yl)methyl)carbamate

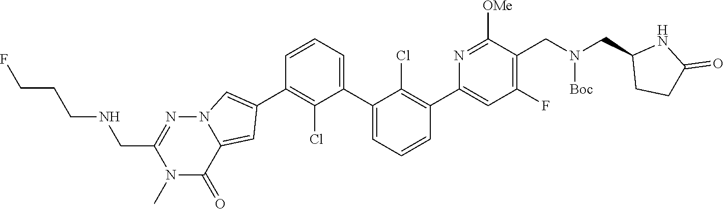 [SMILES: COc1nc(-c2cccc(-c3cccc(-c4cc5c(=O)n(C)c(CNCCCF)nn5c4)c3Cl)c2Cl)cc(F)c1CN(C[C@@H]1CCC(=O)N1)C(=O)OC(C)(C)C]

A mixture of tert-butyl N-[[6-[2-chloro-3-[2-chloro-3-(2-formyl-3-methyl-4-oxo-pyrrolo[2,1-f1][1,2,4]triazin-6-yl)phenyl]phenyl]-4-fluoro-2-methoxy-3-pyridyl]methyl]-N-[[(2S)-5-oxopyrrolidin-2-yl]methyl]carbamate (0.25 g, 0.3 mmol), 3-fluoropropan-1-amine as hydrochloride salt (76 mg, 0.7 mmol) and sodium acetate (96 mg, 1 mmol) in DCM/trimethoxymethane mixture (1:1, 4 mL) was degassed and purged with N₂ 3 times. After stirring for 12 hr at r.t., Sodium triacetoxyborohydride (0.14 g, 0.7 mmol) was added in the mixture and then the mixture was stirred at r.t. for 1 hr under N₂. The reaction mixture was filtered and concentrated to give a residue. The residue was purified by normal phase SiO₂ chromatography (0-15% ethyl acetate/petroleum ether) to afford (S)-tert-butyl ((6-(2,2′-dichloro-3′-(2-(((3-fluoropropyl)amino)methyl)-3-methyl-4-oxo-3,4-dihydropyrrolo[2,1-f1][1,2,4]triazin-6-yl)-[1,1′-biphenyl]-3-yl)-4-fluoro-2-methoxypyridin-3-yl)methyl)((5-oxopyrrolidin-2-yl)methyl)carbamate (0.2 g) as a yellow solid. MS: m/z found 810 [M+H]⁺.

### (S)-6-(2,2′-Dichloro-3′-(4-fluoro-6-methoxy-5-((((5-oxopyrrolidin-2-yl)methyl)amino)methyl)pyridin-2-yl)-[1,1′-biphenyl]-3-yl)-2-(((3-fluoropropyl)amino)methyl)-3-methylpyrrolo[2,1-f][1,2,4]triazin-4(3H)-one

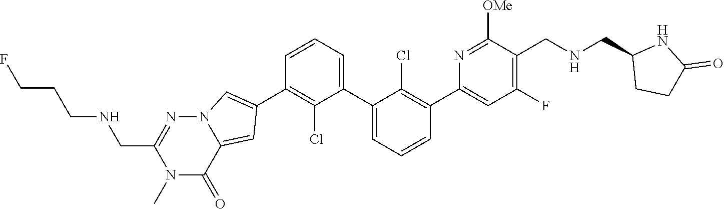 [SMILES: COc1nc(-c2cccc(-c3cccc(-c4cc5c(=O)n(C)c(CNCCCF)nn5c4)c3Cl)c2Cl)cc(F)c1CNC[C@@H]1CCC(=O)N1]

A mixture of tert-butyl N-[[6-[2-chloro-3-[2-chloro-3-[2-[(3-fluoropropylamino)methyl]-3-methyl-4-oxo-pyrrolo[2,1-f][1,2,4]triazin-6-yl]phenyl]phenyl]-4-fluoro-2-methoxy-3-pyridyl]methyl]-N-[[(2S)-5-oxopyrrolidin-2-yl]methyl]carbamate (0.2 g, 0.25 mmol) and trifluoroacetic acid (5 mL) in DCM (5 mL) was stirred at r.t. for 1 hr under N₂. The reaction mixture was filtered and concentrated to give a residue. The residue was purified by reversed phase HPLC to afford (S)-6-(2,2′-dichloro-3′-(4-fluoro-6-methoxy-5-((((5-oxopyrrolidin-2-yl)methyl)amino)methyl)pyridin-2-yl)-[1,1′-biphenyl]-3-yl)-2-(((3-fluoropropyl)amino)methyl)-3-methylpyrrolo[2,1-f][1,2,4]triazin-4(3H)-one as a white solid as a formic acid salt (14.8 mg). MS: m/z found 710 [M+H]⁺; ¹H NMR (400 MHz, Methanol-d₄): δ7.87 (d, J=2 Hz, 1H), 7.69-7.65 (m, 2H), 7.54-7.45 (m, 2H), 7.40 (dd, J=1.6 Hz, J=7.6 Hz, 1H), 7.31-7.28 (m, 2H), 7.16 (d, J=9.2 Hz, 1H), 4.64 (t, J=5.6 Hz, 1H), 4.52 (t, J=6 Hz, 1H), 4.08 (s, 3H), 4.03 (s, 2H), 3.95 (s, 2H), 3.89-3.84 (m, 1H), 3.59 (s, 3H), 2.97 (t, J=7.2 Hz, 2H), 2.79-2.70 (m, 2H), 2.38-2.27 (m, 3H), 2.07-1.94 (m, 2H), 1.87-1.80 (m, 1H). ¹H NMR (400 MHz, DMSO-d₆) δ7.98 (s, 1H), 7.74-7.66 (m, 3H), 7.59-7.55 (m, 1H), 7.52-7.46 (m, 2H), 7.34 (d, J=7.6 Hz, 1H), 7.26-7.24 (m, 2H), 4.59-4.56 (m, 1H), 4.47-4.44 (m, 1H), 3.97 (s, 3H), 3.79-347 (m, 4H), 3.60 (br s, 2H), 3.56-3.53 (m, 3H), 2.72-2.69 (m, 4H), 2.15-2.01 (m, 3H), 1.87-1.77 (m, 2H), 1.69-1.62 (m, 1H).

### Example 509: (S)-5-((6-(2,2′-Dichloro-3′-(3-fluoro-6-methoxy-5-(((((S)-5-oxopyrrolidin-2-yl)methyl)amino)methyl)pyridin-2-yl)-[1,1′-biphenyl]-3-yl)-8-methoxy-3,4-dihydroisoquinolin-2(1H)-yl)methyl)pyrrolidin-2-one

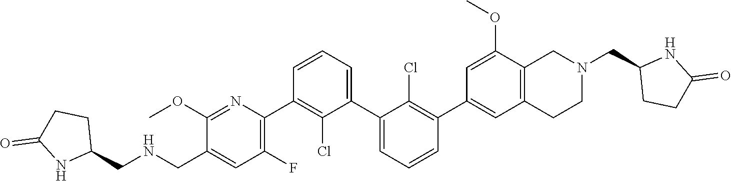 [SMILES: COc1cc(-c2cccc(-c3cccc(-c4nc(OC)c(CNC[C@@H]5CCC(=O)N5)cc4F)c3Cl)c2Cl)cc2c1CN(C[C@@H]1CCC(=O)N1)CC2]

### (S)-5-((6-Bromo-8-methoxy-3,4-dihydroisoquinolin-2(1H)-yl)methyl)pyrrolidin-2-one

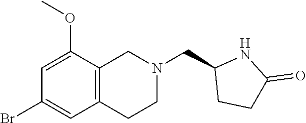 [SMILES: COc1cc(Br)cc2c1CN(C[C@@H]1CCC(=O)N1)CC2]

To the mixture of 6-bromo-8-methoxy-1,2,3,4-tetrahydroisoquinoline (300 mg, 1.24 mmol) in MeCN (100 mL) was added (S)-5-(bromomethyl)pyrrolidin-2-one (405 mg, 2.27 mmol) and potassium carbonate (514 mg, 3.72 mmol). The reaction was stirred for 12 hr at 100° C. The reaction was added H₂O (100 mL) and extracted with EtOAc (200 mL). The organic layer was concentrated under vacuum. The residue was purified by normal phase SiO₂ chromatography (0-100% EtOAc/petroleum ether to 0-10% THF/EtOAc) to afford (S)-5-((6-bromo-8-methoxy-3,4-dihydroisoquinolin-2(1H)-yl)methyl)pyrrolidin-2-one as a yellow solid (260 mg, 56% yield).

### tert-Butyl ((6-(2,2′-dichloro-3′-(8-methoxy-2-(((S)-5-oxopyrrolidin-2-yl)methyl)-1,2,3,4-tetrahydroisoquinolin-6-yl)-[1,1′-biphenyl]-3-yl)-5-fluoro-2-methoxypyridin-3-yl)methyl)(((S)-5-oxopyrrolidin-2-yl)methyl)carbamate

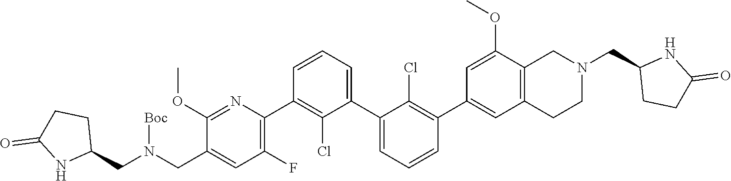 [SMILES: COc1cc(-c2cccc(-c3cccc(-c4nc(OC)c(CN(C[C@@H]5CCC(=O)N5)C(=O)OC(C)(C)C)cc4F)c3Cl)c2Cl)cc2c1CN(C[C@@H]1CCC(=O)N1)CC2]

To the mixture of (S)-5-((6-bromo-8-methoxy-3,4-dihydroisoquinolin-2(1H)-yl)methyl) pyrrolidin-2-one (50 mg, 0.15 mmol) and (S)-tert-butyl ((6-(2,2′-dichloro-3′-(4,4,5,5-tetramethyl-1,3,2-dioxaborolan-2-yl)-[1,1′-biphenyl]-3-yl)-5-fluoro-2-methoxypyridin-3-yl)methyl)((5-oxopyrrolidin-2-yl)methyl)carbamate (134 mg, 0.19 mmol) in dioxane/H₂O (2.2 ml, 10/1) was added potassium phosphate (93.9 mg, 0.44 mmol), followed by di-tert-butyl(cyclopentyl)phosphane; dichloropalladium; iron (9.61 mg, 0.015 mmol) and then the reaction was stirred for 1 hr at 110° C. After cooling, the mixture was added H₂O (10 mL) and extracted with ethyl acetate (20 mL). The organic layer was concentrated under vacuum. The residue was purified by prep-TLC (SiO₂, 20% MeOH/Ethyl acetate) to afford tert-butyl ((6-(2,2′-dichloro-3′-(8-methoxy-2-(((S)-5-oxopyrrolidin-2-yl)methyl)-1,2,3,4-tetrahydroisoquinolin-6-yl)-[1,1′-biphenyl]-3-yl)-5-fluoro-2-methoxypyridin-3-yl)methyl)(((S)-5-oxopyrrolidin-2-yl)methyl)carbamate as a yellow solid (59 mg, 41% yield). MS: m/z found 832 [M+H]⁺.

### (S)-5-((6-(2,2′-Dichloro-3′-(3-fluoro-6-methoxy-5-(((((S)-5-oxopyrrolidin-2-yl)methyl)amino)methyl)pyridin-2-yl)-[1,1′-biphenyl]-3-yl)-8-methoxy-3,4-dihydroisoquinolin-2(1H)-yl)methyl)pyrrolidin-2-one

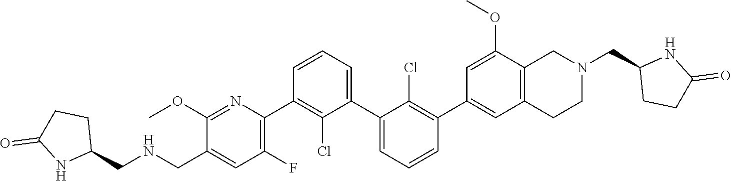 [SMILES: COc1cc(-c2cccc(-c3cccc(-c4nc(OC)c(CNC[C@@H]5CCC(=O)N5)cc4F)c3Cl)c2Cl)cc2c1CN(C[C@@H]1CCC(=O)N1)CC2]

To the mixture of tert-butyl ((6-(2,2′-dichloro-3′-(8-methoxy-2-(((S)-5-oxopyrrolidin-2-yl) methyl)-1,2,3,4-tetrahydroisoquinolin-6-yl)-[1,1′-biphenyl]-3-yl)-5-fluoro-2-methoxypyridin-3-yl)methyl)(((S)-5-oxopyrrolidin-2-yl)methyl)carbamate (54 mg, 0.065 mmol) in DCM (2 mL) was added TFA (3.24 mL, 43.8 mmol) and stirred for 0.5 hr at r.t. The reaction was concentrated under reduced pressure. The residue was purified by reversed phase HPLC to afford ((S)-5-((6-(2,2′-dichloro-3′-(3-fluoro-6-methoxy-5-(((((S)-5-oxopyrrolidin-2-yl)methyl)amino)methyl)pyridin-2-yl)-[1,1′-biphenyl]-3-yl)-8-methoxy-3,4-dihydroisoquinolin-2(1H)-yl)methyl)pyrrolidin-2-one as formic acid salt as a white solid (11.7 mg, 23.0% yield). MS: m/z found 732 [M+H]⁺; ¹H NMR (400 MHz, DMSO-d₆): δ 7.81 (d, J=9.6 Hz, 1H), 7.73 (s, 1H), 7.61-7.46 (m, 6H), 7.40 (dd, J=2.0 Hz, J=7.2 Hz, 1 H), 6.81 (d, J=8.4 Hz, 2H), 3.89 (s, 3H), 3.81 (s, 3H), 3.79-3.77 (m, 1H), 3.72 (d, J=4.4 Hz, 2H), 3.67-3.65 (m, 1H), 3.54 (q, J=16 Hz, J=28 Hz, 2H), 3.30 (s, 2H), 2.85-2.83 (m, 2H), 2.72-2.67 (m, 2H), 2.60-2.56 (m, 3H), 2.20-2.07 (m, 6H), 1.75-1.70 (m, 2H). ¹H NMR (400 MHz, Methanol-d₄): δ 7.66 (d, J=8.8 Hz, 1H), 7.53 (s, 1H), 7.51 (d, J=1.6 Hz, 1H), 7.48-7.44 (m, 1H), 7.44-7.41 (m, 2H), 7.33 (dd, J=2.4 Hz, J=7.8 Hz, 1H), 6.84 (s, 2 H), 4.07-4.04 (m, 1H), 4.00 (s, 3H), 3.91-3.86 (m, 6H), 3.77 (d, J=15.6 Hz, 1H), 3.64 (d, J=16.0 Hz, 1H), 3.37 (s, 1H), 3.00-2.97 (m, 2H), 2.94-2.90 (m, 1H), 2.82-2.63 (m, 5H), 2.41-2.30 (m, 6H), 1.88-1.83 (m, 2H).

### Example 510: 2-(2,2′-Dichloro-3′-(6-methoxy-5-(((((S)-5-oxopyrrolidin-2-yl)methyl)amino)methyl)pyridin-2-yl)-[1,1′-biphenyl]-3-yl)-1-methyl-5-(((((S)-5-oxopyrrolidin-2-yl)methyl)amino)methyl)pyrazolo[1,5-a]pyrimidin-7(1H)-one

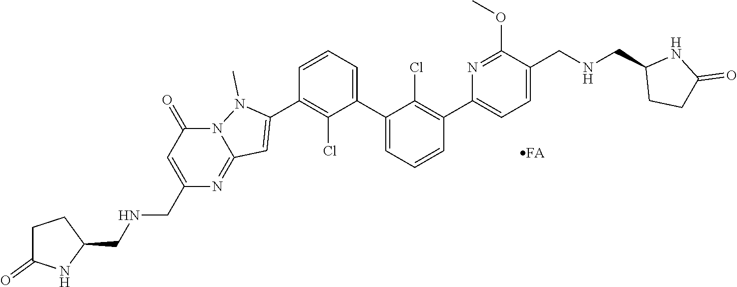

### 2-Bromo-5-(chloromethyl)-1-methylpyrazolo[1,5-a]pyrimidin-7(1H)-one

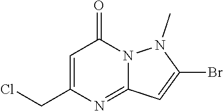

A mixture of 5-bromo-1-methyl-1H-pyrazol-3-amine (0.4 g, 2.27 mmol) and ethyl 4-chloro-3-oxo-butanoate (2.5 mmol, 1.1 eq) in polyphosphoric acid (2 g) was stirred at 100° C. for 3 h. To the mixture was added water (10 mL). The mixture was neutralized with saturated aqueous sodium hydroxide solution. The mixture was filtered. The filter cake was dried under vacuum to afford 2-bromo-5-(chloromethyl)-1-methylpyrazolo[1,5-a]pyrimidin-7(1H)-one as a yellow solid without purification (0.52 g, 82% yield). ¹H NMR (400 MHz, CDCl₃): δ 6.43 (s, 1H), 6.14 (s, 1H), 4.33 (s, 2H), 4.23 (s, 3H).

### (S)-2-Bromo-1-methyl-5-((((5-oxopyrrolidin-2-yl)methyl)amino)methyl)pyrazolo[1,5-a]pyrimidin-7(1H)-one

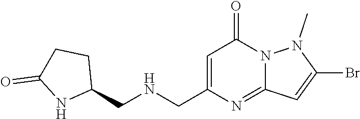 [SMILES: Cn1c(Br)cc2nc(CNC[C@@H]3CCC(=O)N3)cc(=O)n21]

To a mixture of 2-bromo-5-(chloromethyl)-1-methylpyrazolo[1,5-a]pyrimidin-7(1H)-one (0.5 g, 1.81 mmol) and (S)-5-(aminomethyl)-2-pyrrolidinone as a hydrochloride salt (0.41 g, 2.72 mmol) in DMF (12 mL) was added potassium carbonate (0.75 g, 5.43 mmol) in one portion under N₂ atmosphere. The mixture was stirred at 60° C. for 5 hrs. To the mixture was added (S)-5-(aminomethyl)-2-pyrrolidinone as a hydrochloride salt (0.21 g, 1.37 mmol) and potassium carbonate (0.25 g, 1.81 mmol). The mixture was stirred at 60° C. for 5 h. The mixture was used for next step without work-up. (S)-2-bromo-1-methyl-5-((((5-oxopyrrolidin-2-yl)methyl)amino)methyl)pyrazolo[1,5-a]pyrimidin-7(1H)-one was obtained as a yellow suspension in 12 mL of DMF solution. MS: m/z found 354 [M+H]⁺.

### (S)-tert-Butyl ((2-bromo-1-methyl-7-oxo-1,7-dihydropyrazolo[1,5-a]pyrimidin-5-yl)methyl)((5-oxopyrrolidin-2-yl)methyl)carbamate

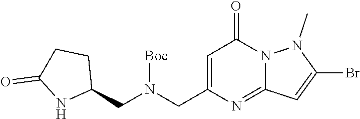 [SMILES: Cn1c(Br)cc2nc(CN(C[C@@H]3CCC(=O)N3)C(=O)OC(C)(C)C)cc(=O)n21]

To the above DMF solution was added triethylamine (0.25 mL, 1.81 mmol) and di-tert-butyl dicarbonate (1.25 mL, 5.42 mmol) in one portion. The mixture was stirred at r.t. for 0.5 h. To the mixture was added water (30 mL) and saturated aqueous brine solution (30 mL). The mixture was extracted with ethyl acetate/THF mixture (1:1, 4×30 mL). The organic phase was dried with anhydrous sodium sulfate, filtered and concentrated. The residue was purified by normal phase SiO₂ chromatography (50˜100% 30% THF in ethyl acetate/petroleum ether to 10% 30% THE in MeOH/ethyl acetate) to afford (S)-tert-butyl ((2-bromo-1-methyl-7-oxo-1,7-dihydropyrazolo[1,5-a]pyrimidin-5-yl)methyl)((5-oxopyrrolidin-2-yl)methyl)carbamate as a yellow gum (0.36 g, 42% yield). ¹H NMR (400 MHz, DMSO-d₆): δ 7.78 (d, J=15.6 Hz, 1H), 5.70 (d, J=5.2 Hz, 1H), 4.31-4.21 (m, 3H), 4.16 (d, J=5.6 Hz, 3H), 3.35-3.25 (m, 2H), 2.16-2.06 (m, 3H), 1.72-1.71 (m, 1H), 1.43-1.35 (m, 9H).

### tert-Butyl N-[[2-[3-[3-[5-[[tert-butoxycarbonyl-[[(2S)-5-oxopyrrolidin-2-yl]methyl]amino]methyl]-6-methoxy-2-pyridyl]-2-chloro-phenyl]-2-chloro-phenyl]-1-methyl-7-oxo-pyrazolo[1,5-a]pyrimidin-5-yl]methyl]-N-[[(2S)-5-oxopyrrolidin-2-yl]methyl]carbamate

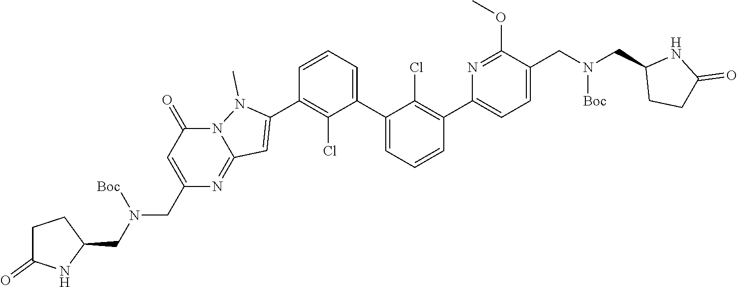 [SMILES: COc1nc(-c2cccc(-c3cccc(-c4cc5nc(CN(C[C@@H]6CCC(=O)N6)C(=O)OC(C)(C)C)cc(=O)n5n4C)c3Cl)c2Cl)ccc1CN(C[C@@H]1CCC(=O)N1)C(=O)OC(C)(C)C]

To a mixture of (S)-tert-butyl ((2-bromo-1-methyl-7-oxo-1,7-dihydropyrazolo[1,5-a]pyrimidin-5-yl)methyl)((5-oxopyrrolidin-2-yl)methyl)carbamate (0.15 g, 0.33 mmol) and (S)-tert-butyl ((6-(2,2′-dichloro-3′-(4,4,5,5-tetramethyl-1,3,2-dioxaborolan-2-yl)-[1,1′-biphenyl]-3-yl)-2-methoxypyridin-3-yl)methyl)((5-oxopyrrolidin-2-yl)methyl)carbamate (0.41 g, 0.59 mmol in dioxane/H₂O mixture (25:3, 5.6 mL) was added 1,1′-bis(diphenylphosphino)ferrocene-palladium(ii) dichloride dichloromethane complex (0.03 g, 0.03 mmol) and potassium carbonate (0.14 g, 0.99 mmol) in one portion under N₂ atmosphere. The mixture was stirred at 110° C. for 4 hrs. The mixture was concentrated. To the residue was added water (20 mL) and saturated aqueous brine solution (20 mL). The mixture was extracted with ethyl acetate/THF mixture (1:1, 40 mL). The organic phase was dried with anhydrous sodium sulfate, filtered and concentrated. The residue was purified by normal phase SiO₂ chromatography (50˜100% ethyl acetate/petroleum ether to 10% MeOH/ethyl acetate) to afford tert-butyl N-[[2-[3-[3-[5-[[tert-butoxycarbonyl-[[(2S)-5-oxopyrrolidin-2-yl]methyl]amino]methyl]-6-methoxy-2-pyridyl]-2-chloro-phenyl]-2-chloro-phenyl]-1-methyl-7-oxo-pyrazolo[1,5-a]pyrimidin-5-yl]methyl]-N-[[(2S)-5-oxopyrrolidin-2-yl]methyl]carbamate as a yellow solid (0.16 g, 37% yield). MS: m/z found 929 [M+H]⁺.

### 2-(2,2′-Dichloro-3′-(6-methoxy-5-(((((S)-5-oxopyrrolidin-2-yl)methyl)amino)methyl)pyridin-2-yl)-[1,1′-biphenyl]-3-yl)-1-methyl-5-(((((S)-5-oxopyrrolidin-2-yl)methyl)amino)methyl)pyrazolo[1,5-a]pyrimidin-7(1H)-one

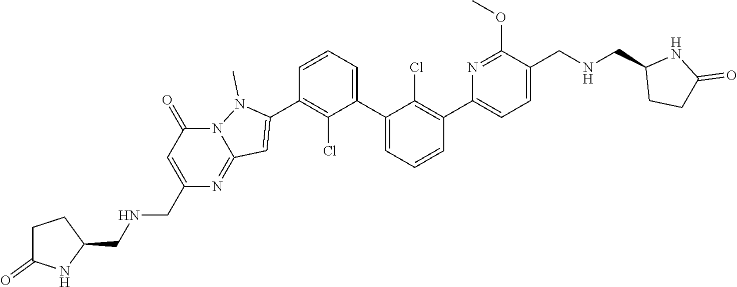 [SMILES: COc1nc(-c2cccc(-c3cccc(-c4cc5nc(CNC[C@@H]6CCC(=O)N6)cc(=O)n5n4C)c3Cl)c2Cl)ccc1CNC[C@@H]1CCC(=O)N1]

To a mixture of tert-butyl N-[[2-[3-[3-[5-[[tert-butoxycarbonyl-[[(2S)-5-oxopyrrolidin-2-yl]methyl]amino]methyl]-6-methoxy-2-pyridyl]-2-chloro-phenyl]-2-chloro-phenyl]-1-methyl-7-oxo-pyrazolo[1,5-a]pyrimidin-5-yl]methyl]-N-[[(2S)-5-oxopyrrolidin-2-yl]methyl]carbamate (0.15 g, 0.16 mmol) in CH₂Cl₂ (0.5 mL) was added trifluoroacetic acid (2 mL, 27 mmol) in one portion. The mixture was stirred at r.t. for 10 min. The mixture was combined with another batch at 10 mg scale. The mixture was concentrated. The residue was purified by reversed phase HPLC to afford 2-(2,2′-dichloro-3′-(6-methoxy-5-(((((S)-5-oxopyrrolidin-2-yl)methyl)amino)methyl)pyridin-2-yl)-[1,1′-biphenyl]-3-yl)-1-methyl-5-(((((S)-5-oxopyrrolidin-2-yl)methyl)amino)methyl)pyrazolo[1,5-a]pyrimidin-7(1H)-one as a formic salt as a light yellow solid (48.2 mg, 38% yield). MS: m/z found 729 [M+H]⁺; ¹H NMR (400 MHz, DMSO-d₆): δ 7.84 (d, J=7.6 Hz, 1H), 7.73-7.69 (m, 6H), 7.58 (t, J=7.6 Hz, 1H), 7.50 (dd, J=7.6 Hz, J=1.6 Hz, 1H), 7.29 (d, J=7.6 Hz, 1H), 6.72 (s, 1H), 6.11 (s, 1H), 3.95 (s, 3H), 3.93 (s, 3H), 3.79-3.71 (m, 2H), 3.69-3.60 (m, 4H), 2.59-2.56 (m, 4H), 2.18-2.08 (m, 6H), 1.75-1.66 (m, 2H). ¹H NMR (400 MHz, Methanol-d₄): δ 7.86 (d, J=6.8 Hz, 1H), 7.72-7.66 (m, 4H), 7.56 (t, J=7.6 Hz, 1H), 7.45 (dd, J=7.6 Hz, J=1.6 Hz, 1H), 7.34 (d, J=7.2 Hz, 1H), 6.65 (s, 1H), 6.15 (s, 1H), 4.15-4.13 (m, 5H), 4.08 (s, 3H), 4.05-3.96 (m, 4H), 3.15-2.92 (m, 4H), 2.44-2.31 (m, 6H), 1.97-1.83 (m, 2H).

### Example 511: 6-(2,2′-Dichloro-3′-(3-fluoro-6-methoxy-5-(((((S)-5-oxopyrrolidin-2-yl)methyl)amino)methyl)pyridin-2-yl)-[1,1′-biphenyl]-3-yl)-2-((((1r,3r)-3-hydroxy-3-methylcyclobutyl)amino)methyl)-3-methylpyrrolo[2,1-f][1,2,4]triazin-4(3H)-one

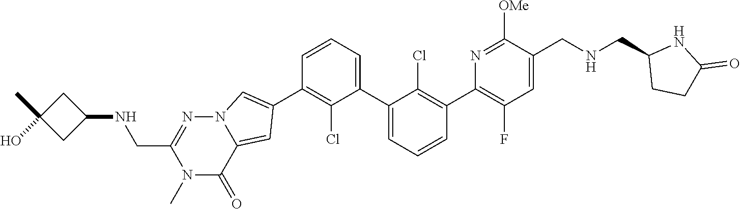

### 6-(3-Bromo-2-chlorophenyl)-2-chloro-5-fluoronicotinic acid

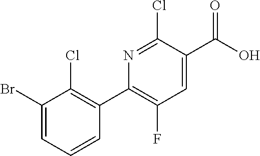 [SMILES: O=C(O)c1cc(F)c(-c2cccc(Br)c2Cl)nc1Cl]

A mixture of 2,6-dichloro-5-fluoronicotinic acid (16 g, 76 mmol), 2-(3-bromo-2-chlorophenyl)-4,4,5,5-tetramethyl-1,3,2-dioxaborolane (24 g, 76 mmol), cesium carbonate (75 g, 229 mmol) and tetrakis(triphenylphosphine)palladium (4.4 g, 3.8 mmol) in dioxane/H₂O mixture (5:1, 420 mL) was stirred at 120° C. for 12 hrs under N₂. The mixture was combined with another two batches at the same scale. The combined mixture was concentrated to remove dioxane. The mixture was diluted with H₂O (1.5 L). To the mixture was added 3 N aqueous HCl solution (200 mL) to adjust its Ph=5, then the mixture was extracted with ethyl acetate (1 L). The organic layers was dried over with anhydrous sodium sulfate, filtered and concentrated to afford 6-(3-bromo-2-chlorophenyl)-2-chloro-5-fluoronicotinic acid (80 g, crude) as a yellow solid without purification. MS: m/z found 366 [M+H]⁺.

### Methyl 6-(3-bromo-2-chlorophenyl)-2-chloro-5-fluoronicotinate

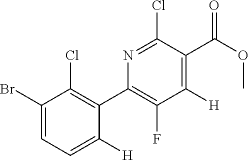 [SMILES: COC(=O)c1cc(F)c(-c2cccc(Br)c2Cl)nc1Cl]

A mixture of 6-(3-bromo-2-chlorophenyl)-2-chloro-5-fluoronicotinic acid (25 g, crude) and thionyl dichloride (7.5 mL, 0.1 mol) in MeOH (250 mL) was degassed and purged with N₂ for 3 times, then the mixture was stirred at 60° C. for 4 hrs under N₂. The reaction mixture was quenched by H₂O (100 mL) at 0° C., then extracted with ethyl acetate (200 mL). The organic layers were washed with saturated aqueous brine solution (200 mL), dried over with anhydrous sodium sulfate, filtered and concentrated to give a residue. The residue was purified by normal phase SiO₂ chromatography (0-10% ethyl acetate/petroleum ether) to afford methyl 6-(3-bromo-2-chlorophenyl)-2-chloro-5-fluoronicotinate was obtained as a yellow solid (15 g, 40%/two steps). ¹H NMR (400 MHz, DMSO-d₆): δ8.42 (d, J=8.4 Hz, 1H), 7.96 (d, J=8.4 Hz, 1H), 7.69 (d, J=7.6 Hz, 1H), 7.44 (t, J=7.6 Hz, 1H), 3.90 (s, 3H).

### 6-(3-Bromo-2-chlorophenyl)-5-fluoro-2-methoxynicotinic acid

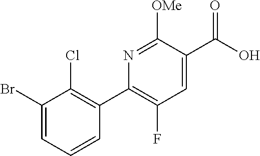 [SMILES: COc1nc(-c2cccc(Br)c2Cl)c(F)cc1C(=O)O]

A mixture of methyl 6-(3-bromo-2-chlorophenyl)-2-chloro-5-fluoronicotinate (10 g, 26 mmol) and sodium methoxide (9.5 g, 53 mmol, 30% in MeOH) in THE (180 mL) was degassed and purged with N₂ for 3 times, then the mixture was stirred at 60° C. for 5 hr under N₂. The reaction mixture was concentrated to give a residue. To the residue was added H₂O (100 ml), then the mixture was extracted with ethyl acetate (100 mL). The organic layers were dried over with anhydrous sodium sulfate, filtered under reduced pressure to afford 6-(3-bromo-2-chlorophenyl)-5-fluoro-2-methoxynicotinic acid as a yellow oil (1.4 g, 15%) without purification. MS: m/z found 362 [M+H]⁺.

### 6-(3-Bromo-2-chlorophenyl)-5-fluoro-2-methoxynicotinic (isobutyl carbonic) anhydride

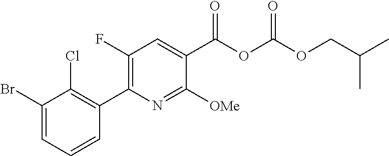 [SMILES: COc1nc(-c2cccc(Br)c2Cl)c(F)cc1C(=O)OC(=O)OCC(C)C]

A mixture of 6-(3-bromo-2-chlorophenyl)-5-fluoro-2-methoxynicotinic acid (7 g, 19.5 mmol), 2-methylpropyl carbonochloridate (2.6 mL, 20 mmol) and triethylamine (8.1 mL, 58 mmol) in THE (70 mL) was degassed and purged with N₂ 3 times, and then the mixture was stirred at 0° C. for 0.5 hrs under N₂. The reaction mixture was filtered to afford 6-(3-bromo-2-chlorophenyl)-5-fluoro-2-methoxynicotinic (isobutyl carbonic) anhydride (8.9 g, crude) as a THE solution (70 mL). The crude product was used directly without further purification.

### (6-(3-Bromo-2-chlorophenyl)-5-fluoro-2-methoxypyridin-3-yl)methanol

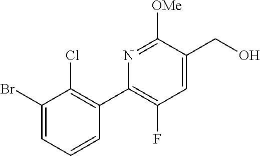 [SMILES: COc1nc(-c2cccc(Br)c2Cl)c(F)cc1CO]

To the above solution of 6-(3-bromo-2-chlorophenyl)-5-fluoro-2-methoxynicotinic (isobutyl carbonic) anhydride in THE (70 mL) was added dropwise sodium borohydride (1.4 g, 38 mmol) in H₂O (10 mL). The mixture was stirred at 0° C. for 0.5 hrs under N₂. H₂O (20 mL) was added to the mixture and stirred at 0° C. for 0.5 hrs, then the mixture was extracted with ethyl acetate (50 mL×3). The organic layers was dried over with anhydrous sodium sulfate, filtered and concentrated to afford (6-(3-bromo-2-chlorophenyl)-5-fluoro-2-methoxypyridin-3-yl)methanol (3 g, 44% yield) as a white solid. ¹H NMR (400 MHz, DMSO-d₆): δ7.91 (d, J=8 Hz, 1H), 7.21 (d, J=9.2 Hz, 1H), 7.53 (d, J=4.8 Hz, 1H), 7.42 (t, J=8 Hz, 1H), 5.46 (t, J=5.6 Hz, 1H), 4.51 (d, J=4.8 Hz, 2H), 3.86 (s, 3H).

### 6-(3-Bromo-2-chlorophenyl)-5-fluoro-2-methoxynicotinaldehyde

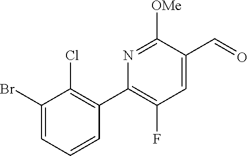 [SMILES: COc1nc(-c2cccc(Br)c2Cl)c(F)cc1C=O]

A mixture of (6-(3-bromo-2-chlorophenyl)-5-fluoro-2-methoxypyridin-3-yl)methanol (3 g, 8.7 mmol) and manganese dioxide (9. g, 104 mmol) in DCM (50 mL) was stirred at 30° C. for 12 hrs under N₂. The reaction mixture was filtered and the filtered was concentrated to afford 6-(3-bromo-2-chlorophenyl)-5-fluoro-2-methoxynicotinaldehyde as a white solid (2.7 g, 90% yield) without purification. ¹H NMR (400 MHz, DMSO-d₆): δ10.29 (s, 1H), 8.17 (d, J=8.4 Hz, 1H), 7.98 (d, J=8 Hz, 1H), 7.65 (d, J=7.6 Hz, 1H), 7.50 (t, J=8 Hz, 1H), 4.04 (s, 3H).

### 6-(2-Chloro-3-(4,4,5,5-tetramethyl-1,3,2-dioxaborolan-2-yl)phenyl)-5-fluoro-2-methoxynicotinaldehyde

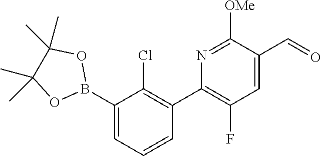 [SMILES: COc1nc(-c2cccc(B3OC(C)(C)C(C)(C)O3)c2Cl)c(F)cc1C=O]

A mixture of 6-(3-bromo-2-chlorophenyl)-5-fluoro-2-methoxynicotinaldehyde (2.5 g, 7.3 mmol), bis(pinacolato)diboron (4.6 g, 18 mmol), 1,1′-bis(diphenylphosphino)ferrocene-palladium(ii) dichloride dichloromethane complex (0.3 g, 0.36 mmol) and potassium acetate (2 g, 22 mmol) in dioxane (60 mL) was degassed and purged with N₂ for 3 times, then the mixture was stirred at 95° C. for 12 hrs under N₂. The reaction mixture was filtered and concentrated to give a residue. The residue was purified by normal phase SiO₂ chromatography (0-10% ethyl acetate/petroleum ether) to afford 6-(2-chloro-3-(4,4,5,5-tetramethyl-1,3,2-dioxaborolan-2-yl)phenyl)-5-fluoro-2-methoxynicotinaldehyde as a yellow solid (2.7 g, 95% yield). ¹H NMR (400 MHz, DMSO-d₆): δ10.25 (s, 1H), 8.11 (d, J=8.4 Hz, 1H), 7.78 (d, J=7.6 Hz, 1H), 7.64 (d, J=7.6 Hz, 1H), 7.54 (t, J=7.6 Hz, 1H), 4.01 (s, 3H), 1.34 (s, 12H).

### 6-(3′-Bromo-2,2′-dichloro-[1,1′-biphenyl]-3-yl)-5-fluoro-2-methoxynicotinaldehyde

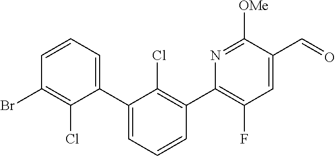 [SMILES: COc1nc(-c2cccc(-c3cccc(Br)c3Cl)c2Cl)c(F)cc1C=O]

A mixture of 6-(2-chloro-3-(4,4,5,5-tetramethyl-1,3,2-dioxaborolan-2-yl)phenyl)-5-fluoro-2-methoxynicotinaldehyde (2.2 g, 5.6 mmol), 1-bromo-2-chloro-3-iodobenzene (5.4 g, 17 mmol), potassium carbonate (2.3 g, 17 mmol), tetrakis(triphenylphosphine)palladium (0.32 g, 0.28 mmol) in dioxane/H₂O mixture (7:1, 80 mL) was degassed and purged with N₂ for 3 times, then the mixture was stirred at 80° C. for 3 hr under N₂. The reaction mixture was concentrated to remove dioxane. The mixture was diluted with H₂O (30 mL) and extracted with ethyl acetate (25 mL×2). The combined organic layers were washed with H₂O (25 mL×2), dried over with anhydrous sodium sulfate, filtered and concentrated to give a residue.

The residue was purified by normal phase SiO₂ chromatography (0-10% ethyl acetate/petroleum ether) to afford 6-(3′-bromo-2,2′-dichloro-[1,1′-biphenyl]-3-yl)-5-fluoro-2-methoxynicotinaldehyde as a brown solid (0.6 g, 23% yield). MS: m/z found 456 [M+H]⁺.

### (S)-5-((((6-(3′-Bromo-2,2′-dichloro-[1,1′-biphenyl]-3-yl)-5-fluoro-2-methoxypyridin-3-yl)methyl)amino)methyl)pyrrolidin-2-one

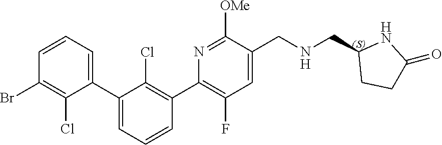

A mixture of 6-(3′-bromo-2,2′-dichloro-[1,1′-biphenyl]-3-yl)-5-fluoro-2-methoxynicotinaldehyde (0.6 g, 1.3 mmol), (S)-5-(aminomethyl)pyrrolidin-2-one as hydrochloride salt (0.3 g, 2 mmol) and sodium acetate (0.32 g, 4 mmol) in DCM (10 mL) was degassed and purged with N₂ for 3 times, then the mixture was stirred at r.t. for 12 hr under N₂. Sodium triacetyloxyborohydride (0.56 g, 2.6 mmol) was added to the mixture and stirred at r.t. for 1 hr under N₂ to afford (S)-5-((((6-(3′-bromo-2,2′-dichloro-[1,1′-biphenyl]-3-yl)-5-fluoro-2-methoxypyridin-3-yl)methyl)amino)methyl)pyrrolidin-2-one as a DCM solution. MS: m/z found 554 [M+H]⁺. The crude product was used directly without further purification.

### (S)-tert-Butyl ((6-(3′-bromo-2,2′-dichloro-[1,1′-biphenyl]-3-yl)-5-fluoro-2-methoxypyridin-3-yl)methyl)((5-oxopyrrolidin-2-yl)methyl)carbamate

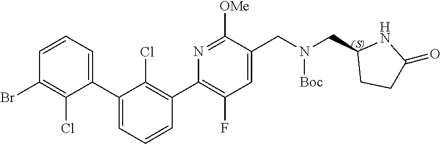

To the above solution of (S)-5-((((6-(3′-bromo-2,2′-dichloro-[1,1′-biphenyl]-3-yl)-5-fluoro-2-methoxypyridin-3-yl)methyl)amino)methyl)pyrrolidin-2-one in DCM (10 mL) was added di-tert-butyl dicarbonate (1 mL, 4.5 mmol), triethylamine (0.63 mL, 4.52 mmol). The mixture was stirred at r.t. for 1.5 hrs under N₂. The mixture was filtered to give the filtrate. The filtrate was concentrated to give the residue. The residue was purified by normal phase SiO₂ chromatography (0-80% ethyl acetate/petroleum ether) to afford (S)-tert-butyl ((6-(3′-bromo-2,2′-dichloro-[1,1′-biphenyl]-3-yl)-5-fluoro-2-methoxypyridin-3-yl)methyl)((5-oxopyrrolidin-2-yl)methyl)carbamate (0.8 g, 67% yield) as a yellow solid. ¹H NMR (400 MHz, DMSO-d₆): δ 7.89-7.86 (m, 1H), 7.78-7.76 (m, 1H), 7.61-7.57 (m, 2H), 7.49-7.40 (m, 3H), 4.40 (q, J=10.8 Hz, 2H), 4.05-4.03 (m, 1H), 4.02 (s, 3H), 3.32-3.31 (m, 2H), 2.16-2.07 (m, 3H), 1.73-1.71 (m, 1H), 1.44-1.30 (m, 9H).

### (S)-tert-Butyl ((6-(2,2′-dichloro-3′-(4,4,5,5-tetramethyl-1,3,2-dioxaborolan-2-yl)-[1,1′-biphenyl]-3-yl)-5-fluoro-2-methoxypyridin-3-yl)methyl)((5-oxopyrrolidin-2-yl)methyl)carbamate

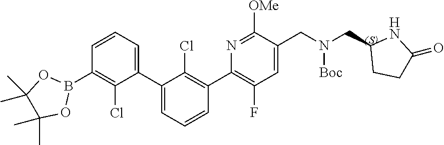

To a mixture of (S)-tert-butyl ((6-(3′-bromo-2,2′-dichloro-[1,1′-biphenyl]-3-yl)-5-fluoro-2-methoxypyridin-3-yl)methyl)((5-oxopyrrolidin-2-yl)methyl)carbamate (0.6 g, 0.92 mmol), bis(pinacolato)diboron (0.7 g, 2.8 mmol) and potassium acetate (0.23 mg, 2.3 mmol) in dioxane (8 mL) was added 1,1′-bis(diphenylphosphino)ferrocene-palladium(ii) dichloride dichloromethane complex (37.7 mg, 46 umol), then the mixture was stirred at 110° C. for 6 hr under N₂. The mixture was filtered to give the filtrate. The filtrate was concentrated to give the residue. The residue was purified by normal phase SiO₂ chromatography (0-80% ethyl acetate/petroleum ether) to afford (S)-tert-butyl ((6-(2,2′-dichloro-3′-(4,4,5,5-tetramethyl-1,3,2-dioxaborolan-2-yl)-[1,1′-biphenyl]-3-yl)-5-fluoro-2-methoxypyridin-3-yl)methyl)((5-oxopyrrolidin-2-yl)methyl)carbamate as a yellow solid (0.55 g, 85% yield). MS: m/z found 700 [M+H]⁺.

### 6-Bromo-3-methyl-4-oxo-3,4-dihydropyrrolo[2,1-f][1,2,4]triazine-2-carbaldehyde

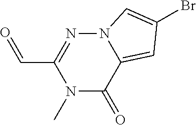

A mixture of 6-bromo-2-(hydroxymethyl)-3-methylpyrrolo[2,1-f][1,2,4]triazin-4(3H)-one (0.4 g, 1.5 mmol) and Dess-Martin (0.98 g, 2.3 mmol) in DCM (10 mL) was stirred at r.t. for 2 hrs under N₂. The reaction mixture was filtered and concentrated under reduced pressure to give a residue. The residue was purified by normal phase SiO₂ chromatography (0-20% ethyl acetate/petroleum ether) to afford 6-bromo-3-methyl-4-oxo-3,4-dihydropyrrolo[2,1-f][1,2,4]triazine-2-carbaldehyde (0.3 g, 75% yield) as a white solid. ¹H NMR (400 MHz, DMSO-d₆): δ 9.46 (s, 1H), 8.09 (s, 1H), 7.16 (s, 1H), 3.61 (s, 3H).

### 6-Bromo-2-((((1r,3r)-3-hydroxy-3-methylcyclobutyl)amino)methyl)-3-methylpyrrolo[2,1-f][1,2,4]triazin-4(3H)-one

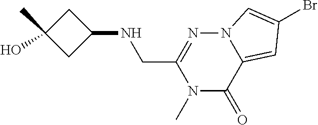

A mixture of 6-bromo-3-methyl-4-oxo-3,4-dihydropyrrolo[2,1-f][1,2,4]triazine-2-carbaldehyde (0.66 g, 2.6 mmol), (1r,3r)-3-amino-1-methylcyclobutanol as hydrochloride salt (0.53 g, 3.9 mmol), sodium acetate (0.63 g, 7.7 mmol) and sodium triacetyloxyborohydride (1.1 g, 5.1 mmol) in DCM (30 mL) was degassed and purged with N₂ for 3 times. Then the mixture was stirred at r.t for 6 hrs under N₂ to afford 6-bromo-2-((((1r,3r)-3-hydroxy-3-methylcyclobutyl)amino)methyl)-3-methylpyrrolo[2,1-f][L,2,4]triazin-4(3H)-one as a DCM solution (30 mL). MS: m/z found 341 [M+H]⁺. The crude product was used directly without further purification.

### tert-Butyl ((6-bromo-3-methyl-4-oxo-3,4-dihydropyrrolo[2,1-f][1,2,4]triazin-2-yl)methyl)((1r,3r)-3-hydroxy-3-methylcyclobutyl)carbamate

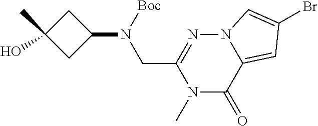

To the above solution of 6-bromo-2-((((1r,3r)-3-hydroxy-3-methylcyclobutyl)amino)methyl)-3-methylpyrrolo[2,1-f][1,2,4]triazin-4(3H)-one in DCM (30 mL) was added di-tert-butyl dicarbonate (1.47 mL, 6.4 mmol) and triethylamine (0.9 mL, 6.4 mmol). The mixture was degassed and purged with N₂ for 3 times and stirred at r.t. for 1 h under N₂. To the mixture was added H₂O (25 mL), then mixture was extracted with ethyl acetate (150 mL×2), dried over with anhydrous sodium sulfate, filtered and concentrated under reduced pressure to give a residue. The residue was purified by normal phase SiO₂ chromatography (0-100% ethyl acetate/petroleum ether) to afford tert-butyl ((6-bromo-3-methyl-4-oxo-3,4-dihydropyrrolo[2,1-f][1,2,4]triazin-2-yl)methyl)((1r,3r)-3-hydroxy-3-methylcyclobutyl)carbamate as a yellow solid (0.15 g, 13% yield). ¹H NMR (400 MHz, DMSO-d₆): δ 7.79 (s, 1H), 7.04 (s, 1H), 4.77 (s, 1H), 4.54 (s, 2H), 3.63 (s, 1H), 3.47 (s, 3H), 2.15 (d, J=8.8 Hz, 4H), 1.41 (br s, 9H), 1.27 (s, 3H).

### tert-Butyl ((6-(3′-(2-(((tert-butoxycarbonyl)((1r,3r)-3-hydroxy-3-methylcyclobutyl)amino)methyl)-3-methyl-4-oxo-3,4-dihydropyrrolo[2,1-f][1,2,4]triazin-6-yl)-2,2′-dichloro-[1,1′-biphenyl]-3-yl)-5-fluoro-2-methoxypyridin-3-yl)methyl)(((S)-5-oxopyrrolidin-2-yl)methyl)carbamate

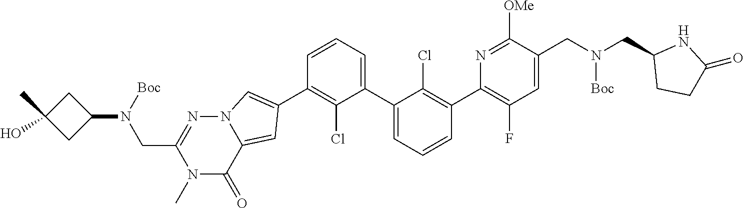

To a mixture of tert-butyl ((6-bromo-3-methyl-4-oxo-3,4-dihydropyrrolo[2,1-f][1,2,4]triazin-2-yl)methyl)((1r,3r)-3-hydroxy-3-methylcyclobutyl)carbamate (0.11 g, 0.26 mmol) and (S)-tert-butyl ((6-(2,2′-dichloro-3′-(4,4,5,5-tetramethyl-1,3,2-dioxaborolan-2-yl)-[1,1′-biphenyl]-3-yl)-5-fluoro-2-methoxypyridin-3-yl)methyl)((5-oxopyrrolidin-2-yl)methyl)carbamate (0.15 g, 0.21 mmol) in dioxane/H₂O mixture (5:1, 1.8 mL) was added [1,1′-Bis(di-tert-butylphosphino)ferrocene]dichloropalladium(II) (7 mg, 11 umol) and potassium phosphate (0.14 mg, 0.64 mmol). The mixture was stirred at 110° C. for 0.5 hrs under N₂. The mixture was combined with another batch at 50 mg scale. To the combined mixture was added water (25 mL). The mixture was extracted with ethyl acetate (50 mL×2). The combined organic phase was dried with anhydrous sodium sulfate, filtered and concentrated to give the residue. The residue was purified by normal phase SiO₂ chromatography (0-90% ethyl acetate/petroleum ether) to afford tert-butyl ((6-(3′-(2-(((tert-butoxycarbonyl)((1r,3r)-3-hydroxy-3-methylcyclobutyl)amino)methyl)-3-methyl-4-oxo-3,4-dihydropyrrolo[2,1-f1][1,2,4]triazin-6-yl)-2,2′-dichloro-[1,1′-biphenyl]-3-yl)-5-fluoro-2-methoxypyridin-3-yl)methyl)(((S)-5-oxopyrrolidin-2-yl)methyl)carbamate as a yellow solid (50 mg, 44% purity). MS: m/z found 934 [M+H]⁺.

### 6-(2,2′-Dichloro-3′-(3-fluoro-6-methoxy-5-(((((S)-5-oxopyrrolidin-2-yl)methyl)amino)methyl)pyridin-2-yl)-[1,1′-biphenyl]-3-yl)-2-((((1r,3r)-3-hydroxy-3-methylcyclobutyl)amino)methyl)-3-methylpyrrolo[2,1-f][1,2,4]triazin-4(3H)-one

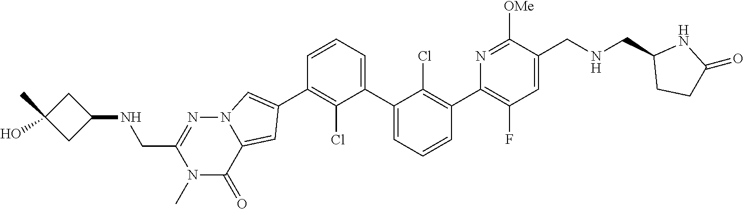

To a mixture of tert-butyl ((6-(3′-(2-(((tert-butoxycarbonyl)((1r,3r)-3-hydroxy-3-methylcyclobutyl)amino)methyl)-3-methyl-4-oxo-3,4-dihydropyrrolo[2,1-f][1,2,4]triazin-6-yl)-2,2′-dichloro-[1,1′-biphenyl]-3-yl)-5-fluoro-2-methoxypyridin-3-yl)methyl)(((S)-5-oxopyrrolidin-2-yl)methyl)carbamate (50 mg, 54 umol, 44% purity) in DCM (0.5 mL) was added trifluoroacetic acid (0.6 mL). The mixture was stirred at r.t. for 0.5 hrs under N₂. The mixture was concentrated to give the residue. The residue was purified by reversed phase HPLC to afford 6-(2,2′-dichloro-3′-(3-fluoro-6-methoxy-5-(((((S)-5-oxopyrrolidin-2-yl)methyl)amino)methyl)pyridin-2-yl)-[1,1′-biphenyl]-3-yl)-2-((((1r,3r)-3-hydroxy-3-methylcyclobutyl)amino)methyl)-3-methylpyrrolo[2,1-f][1,2,4]triazin-4(3H)-one as formic acid salt as a white solid (4 mg, 9.6% yield). MS: m/z found 734 [M+H]⁺; ¹H NMR (400 MHz, D₂O): δ7.92 (d, J=2 Hz, 1H), 7.75 (d, J=8 Hz, 1H), 7.64 (dd, J=6.8 Hz, J=2 Hz, 1H), 7.55-7.44 (m, 4H), 7.34-7.33 (m, 2H), 4.36 (s, 2H), 4.29 (s, 2H), 4.10-4.06 (m, 2H), 3.94 (s, 3H), 3.86 (s, 3H), 3.21 (d, J=6.4 Hz, 2H), 2.49-2.31 (m, 7H), 1.68-1.83 (m, 1H), 1.36 (s, 3H). ¹H NMR (400 MHz, Methanol-d₄): δ7.90 (d, J=2 Hz, 1H), 7.73 (d, J=9.2 Hz, 1H), 7.66 (dd, J=7.2 Hz, J=1.6 Hz, 1H), 7.55-7.54 (m, 2H), 7.50-7.43 (m, 2H), 7.34-7.30 (m, 2H), 4.22 (br s, 2H), 4.08-4.04 (m, 5H), 3.95 (br s, 2H), 3.56 (s, 3H), 2.97 (br s, 2H), 2.46-2.36 (m, 5H), 2.19-2.16 (m, 2H), 1.94-1.88 (m, 1H), 1.42 (s, 3H). ¹H NMR (400 MHz, DMSO-d₆): δ 8.00 (d, J=2 Hz, 1H), 7.76 (dd, J=6.8 Hz, J=1.2 Hz, 1H), 7.61-7.60 (m, 2H), 7.53-7.50 (m, 2H), 7.40-7.35 (m, 1H), 7.31-7.28 (m, 1H), 3.93 (s, 2H), 3.80-3.62 (m, 2H), 3.54-3.47 (m, 2H), 3.30 (s, 3H), (overlapped with solvent peak), 2.21-2.15 (m, 6H), 1.77-1.68 (m, 2H), 1.27 (s, 3H), 1.15 (s, 1H).

### Example 512: (S)-Isopropyl 2-(((6-(2,2′-dichloro-3′-(4-chloro-6-methoxy-5-((((5-oxopyrrolidin-2-yl)methyl)amino)methyl)pyridin-2-yl)-[1,1′-biphenyl]-3-yl)-3-methyl-4-oxo-3,4-dihydropyrrolo[2,1-f][1,2,4]triazin-2-yl)methyl)amino)-2-methylpropanoate

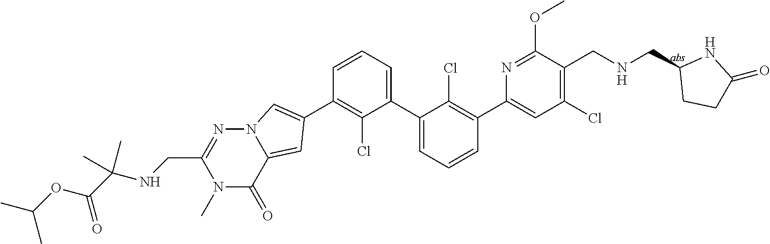 [SMILES: COc1nc(-c2cccc(-c3cccc(-c4cc5c(=O)n(C)c(CNC(C)(C)C(=O)OC(C)C)nn5c4)c3Cl)c2Cl)cc(Cl)c1CNC[C@@H]1CCC(=O)N1]

### Isopropyl 2-amino-2-methylpropanoate as hydrochloride salt

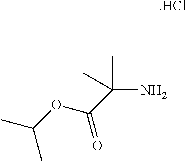

To a mixture of 2-amino-2-methylpropanoic acid (1 g, 9.70 mmol) in propan-2-ol (6 mL, 78.38 mmol) was added SOCl₂ (1.06 mL, 14.5 mmol) under N₂. The mixture was stirred at 95° C. for 24 h. The mixture was concentrated to afford isopropyl 2-amino-2-methylpropanoate as hydrochloride salt (1.7 g, crude). The crude product was used directly without future purification. ¹H NMR (400 MHz, D₂O): δ8.63 (s, 3H), 4.93-4.82 (m, 1H), 1.38 (s, 6H), 1.15 (d, J=6 Hz, 6H).

### (S)-Isopropyl 2-(((6-(3′-(5-(((tert-butoxycarbonyl)((5-oxopyrrolidin-2-yl)methyl)amino)methyl)-4-chloro-6-methoxypyridin-2-yl)-2,2′-dichloro-[1,1′-biphenyl]-3-yl)-3-methyl-4-oxo-3,4-dihydropyrrolo[2,1-f][1,2,4]triazin-2-yl)methyl)amino)-2-methylpropanoate

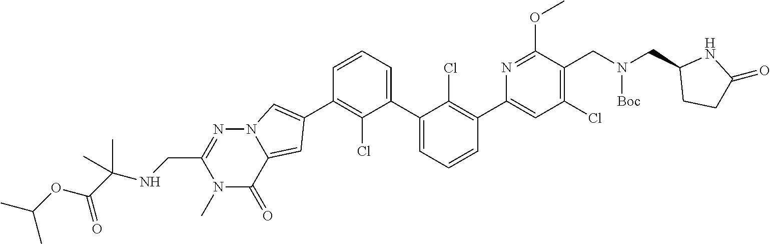 [SMILES: COc1nc(-c2cccc(-c3cccc(-c4cc5c(=O)n(C)c(CNC(C)(C)C(=O)OC(C)C)nn5c4)c3Cl)c2Cl)cc(Cl)c1CN(C[C@@H]1CCC(=O)N1)C(=O)OC(C)(C)C]

To a mixture of (S)-tert-butyl ((4-chloro-6-(2,2′-dichloro-3′-(2-formyl-3-methyl-4-oxo-3,4-dihydropyrrolo[2,1-f1][1,2,4]triazin-6-yl)-[1,1′-biphenyl]-3-yl)-2-methoxypyridin-3-yl)methyl)((5-oxopyrrolidin-2-yl)methyl)carbamate (20 mg, 26.1 umol), isopropyl 2-amino-2-methylpropanoate as hydrochloric acid salt (15.2 mg, 104 umol) in DCM/trimethyl orthoformate (3:1, 1.6 mL) was added sodium acetate (12.8 mg, 157 umol) then the mixture was stirred at r.t. for 12 h under N₂, after that sodium cyanoborohydride (4.92 mg, 78.3 umol) was added to the mixture, and then the mixture was stirred at r.t for 0.5 h under N₂. The mixture was combined with another two batches at the 20 mg and 10 mg scale. The mixture was concentrated to give the residue. The residue was purified by prep-TLC (SiO₂, EtOAc:MeOH=5:1) to afford (S)-isopropyl 2-(((6-(3′-(5-(((tert-butoxycarbonyl)((5-oxopyrrolidin-2-yl)methyl)amino)methyl)-4-chloro-6-methoxypyridin-2-yl)-2,2′-dichloro-[1,1′-biphenyl]-3-yl)-3-methyl-4-oxo-3,4-dihydropyrrolo[2,1-f][1,2,4]triazin-2-yl)methyl)amino)-2-methylpropanoate (35 mg) as a white solid. MS: m/z found 894 [M+H]⁺.

### (S)-Isopropyl 2-(((6-(2,2′-dichloro-3′-(4-chloro-6-methoxy-5-((((5-oxopyrrolidin-2-yl)methyl)amino)methyl)pyridin-2-yl)-[1,1′-biphenyl]-3-yl)-3-methyl-4-oxo-3,4-dihydropyrrolo[2,1-f][1,2,4]triazin-2-yl)methyl)amino)-2-methylpropanoate

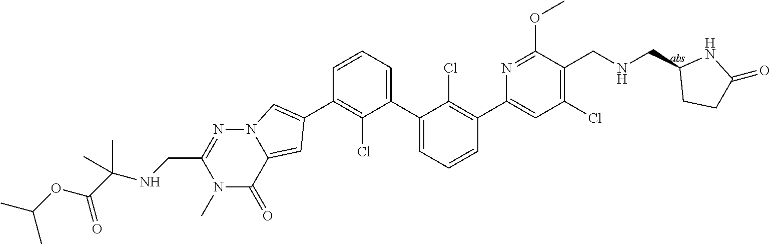 [SMILES: COc1nc(-c2cccc(-c3cccc(-c4cc5c(=O)n(C)c(CNC(C)(C)C(=O)OC(C)C)nn5c4)c3Cl)c2Cl)cc(Cl)c1CNC[C@@H]1CCC(=O)N1]

The (S)-isopropyl 2-(((6-(3′-(5-(((tert-butoxycarbonyl)((5-oxopyrrolidin-2-yl)methyl)amino)methyl)-4-chloro-6-methoxypyridin-2-yl)-2,2′-dichloro-[1,1′-biphenyl]-3-yl)-3-methyl-4-oxo-3,4-dihydropyrrolo[2,1-f][1,2,4]triazin-2-yl)methyl)amino)-2-methylpropanoate (30 mg, 0.03 mmol) in DCM (3 mL) was added TFA (3 mL, 40.5 mmol), after that the mixture was stirred at r.t. for 0.5 h under N₂. The mixture was concentrated to give the residue. The residue was purified by reverse phase HPLC to afford (S)-isopropyl 2-(((6-(2,2′-dichloro-3′-(4-chloro-6-methoxy-5-((((5-oxopyrrolidin-2-yl)methyl)amino)methyl)pyridin-2-yl)-[1,1′-biphenyl]-3-yl)-3-methyl-4-oxo-3,4-dihydropyrrolo[2,1-f][1,2,4]triazin-2-yl)methyl)amino)-2-methylpropanoate (7.5 mg, 27% yield) as a white solid. MS: m/z found 794 [M+H]⁺; ¹H NMR (400 MHz, DMSO-d₆): δ 8.00 (d, J=2 Hz, 1H), 7.76-7.70 (m, 3H), 7.59 (t, J=7.2 Hz, 1H), 7.54-7.49 (m, 2H), 7.42 (s, 1H), 7.37 (dd, J₁=1.2 Hz, J₂=7.6 Hz, 1H), 7.26 (d, J=2 Hz, 1H), 4.91-4.85 (m, 1H), 3.99 (s, 3H), 3.89 (s, 2H), 3.77 (d, J=6.8 Hz, 2H), 3.63-3.62 (m, 1H), 3.59 (s, 3H), 3.33 (s, 1H), 2.90 (t, J=7.2 Hz, 1H), 2.16-2.03 (m, 4H), 1.73-1.69 (m, 1H), 1.32 (s, 6H), 1.19 (d, J=6.8 Hz, 6H). ¹H NMR (400 MHz, MeOH-d₆): δ7.81 (d, J=1.6 Hz, 1H), 7.63-7.60 (m, 2H), 7.47 (t, J=7.6 Hz, 1H), 7.42 (t, J=8 Hz, 1H), 7.36 (dd, J₁=1.6 Hz, J₂=7.6 Hz, 1H), 7.31 (s, 1H), 7.25-7.23 (m, 2H), 4.92-4.88 (m, 1H), 4.02 (s, 3H), 3.99 (s, 2H), 3.84 (s, 2H), 3.80-3.79 (m, 1H), 3.60 (s, 3H), 2.69-2.65 (m, 2H), 2.32-2.21 (m, 3H), 1.80-1.79 (m, 1H), 1.37 (s, 6H), 1.19 (d, J=6 Hz, 6H).

### Example 514: (S)-2-(2,2′-Dichloro-3′-(6-methoxy-5-((((5-oxopyrrolidin-2-yl)methyl)amino)methyl)pyridin-2-yl)-[1,1′-biphenyl]-3-yl)-5-(((2-hydroxyethyl)amino)methyl)-1-methylpyrazolo[1,5-a]pyrimidin-7(1H)-one

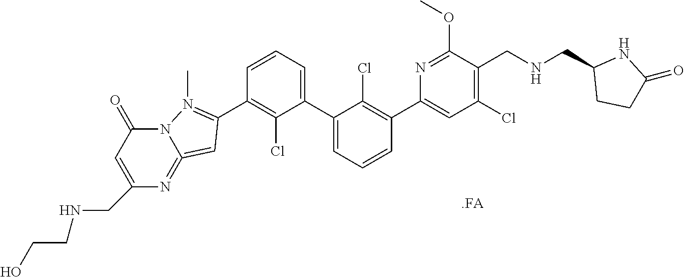

### 2-Bromo-5-(((2-((tert-butyldimethylsilyl)oxy)ethyl)amino)methyl)-1-methylpyrazolo[1,5-a]pyrimidin-7(1H)-oneL

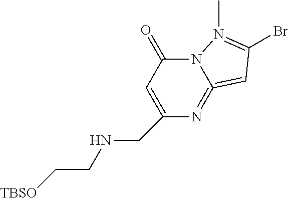

To a mixture of 2-bromo-5-(chloromethyl)-1-methyl-pyrazolo[1,5-a]pyrimidin-7-one (0.35 g, 1.27 mmol) and 2-((tert-butyldimethylsilyl)oxy)ethanamine (0.44 g, 2.53 mmol) in DMF (5 mL) was added potassium carbonate (0.52 g, 3.8 mmol) in one portion under N₂ atmosphere. The mixture was stirred at 60° C. for 4 h. The mixture was used for next step without work-up. 2-bromo-5-[[2-[tert-butyl(dimethyl)silyl]oxyethylamino]methyl]-1-methyl-pyrazolo[1,5-a]pyrimidin-7-one was obtained as a yellow liquid in 5 mL of DMF solution. MS: m/z found 415 [M+H]⁺.

### tert-Butyl ((2-bromo-1-methyl-7-oxo-1,7-dihydropyrazolo[1,5-a]pyrimidin-5-yl)methyl)(2-((tert-butyldimethylsilyl)oxy)ethyl)carbamate

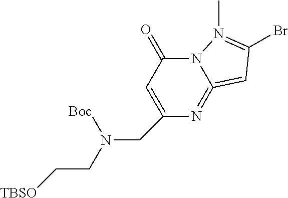

To the above solution was added triethylamine (0.18 mL, 1.26 mmol) and di-tert-butyl dicarbonate (0.73 mL, 3.16 mmol) in one portion. The mixture was stirred at r.t. for 0.5 h. To the mixture was added water (30 mL) and saturated aqueous brine solution (20 mL). The mixture was extracted with ethyl acetate (2×20 mL). The combined organic phase was washed with saturated aqueous brine solution (20 mL), dried with anhydrous sodium sulfate, filtered and concentrated. The residue was purified by normal phase SiO₂ chromatography (0-30% ethyl acetate/petroleum ether) to afford tert-butyl ((2-bromo-1-methyl-7-oxo-1,7-dihydropyrazolo[1,5-a]pyrimidin-5-yl)methyl)(2-((tert-butyldimethylsilyl)oxy)ethyl)carbamate as a yellow gum (0.2 g, 22% yield). ¹H NMR (400 MHz, CDCl₃): δ 6.41 (s, 1H), 5.91-5.83 (m, 1H), 4.24 (s, 2H), 4.20-4.15 (m, 3H), 3.71-3.65 (m, 2H), 3.45-3.38 (m, 2H), 1.35-1.24 (m, 9H), 0.82 (s, 9H), 0.00 (s, 6H).

### (S)-tert-Butyl ((2-(3′-(5-(((tert-butoxycarbonyl)((5-oxopyrrolidin-2-yl)methyl)amino)methyl)-6-methoxypyridin-2-yl)-2,2′-dichloro-[1,1′-biphenyl]-3-yl)-1-methyl-7-oxo-1,7-dihydropyrazolo[1,5-a]pyrimidin-5-yl)methyl)(2-((tert-butyldimethylsilyl)oxy)ethyl)carbamate

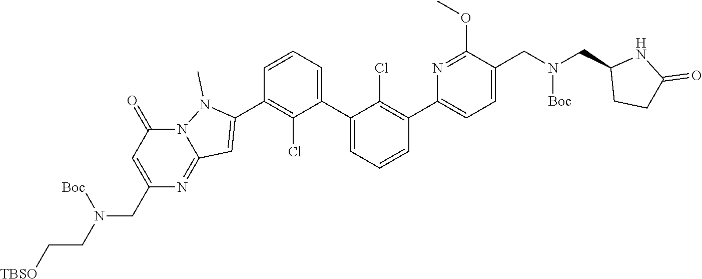 [SMILES: COc1nc(-c2cccc(-c3cccc(-c4cc5nc(CN(CCO[Si](C)(C)C(C)(C)C)C(=O)OC(C)(C)C)cc(=O)n5n4C)c3Cl)c2Cl)ccc1CN(C[C@@H]1CCC(=O)N1)C(=O)OC(C)(C)C]

To a mixture of tert-butyl ((2-bromo-1-methyl-7-oxo-1,7-dihydropyrazolo[1,5-a]pyrimidin-5-yl)methyl)(2-((tert-butyldimethylsilyl)oxy)ethyl)carbamate (0.16 g, 0.31 mmol) and (S)-tert-butyl ((6-(2,2′-dichloro-3′-(4,4,5,5-tetramethyl-1,3,2-dioxaborolan-2-yl)-[1,1′-biphenyl]-3-yl)-2-methoxypyridin-3-yl)methyl)((5-oxopyrrolidin-2-yl)methyl)carbamate (0.32 g, 0.47 mmol) in dioxane/H₂O mixture (5:1, 6 mL) was added potassium carbonate (0.13 g, 0.93 mmol) and 1,1′-bis(diphenylphosphino)ferrocene-palladium(ii) dichloride dichloromethane complex (0.03 g, 0.03 mmol) in one portion under N₂ atmosphere. The mixture was stirred at 110° C. for 5 h. To the mixture was added water (10 mL). The mixture was extracted with ethyl acetate (2×10 mL). The combined organic phase was washed with saturated aqueous brine solution (2×10 mL), dried with anhydrous sodium sulfate, filtered and concentrated. The residue was purified by normal phase SiO₂ chromatography (0-60% 30% THE in ethyl acetate/petroleum ether) to afford (S)-tert-butyl ((2-(3′-(5-(((tert-butoxycarbonyl)((5-oxopyrrolidin-2-yl)methyl)amino)methyl)-6-methoxypyridin-2-yl)-2,2′-dichloro-[1,1′-biphenyl]-3-yl)-1-methyl-7-oxo-1,7-dihydropyrazolo[1,5-a]pyrimidin-5-yl)methyl)(2-((tert-butyldimethylsilyl)oxy)ethyl)carbamate as a yellow solid (0.32 g, 29% yield). MS: m/z found 990 [M+H]⁺.

### (S)-2-(2,2′-dichloro-3′-(6-methoxy-5-((((5-oxopyrrolidin-2-yl)methyl)amino)methyl)pyridin-2-yl)-[1,1′-biphenyl]-3-yl)-5-(((2-hydroxyethyl)amino)methyl)-1-methylpyrazolo[1,5-a]pyrimidin-7(1H)-one

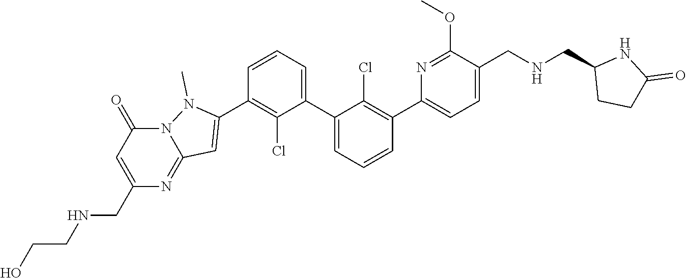 [SMILES: COc1nc(-c2cccc(-c3cccc(-c4cc5nc(CNCCO)cc(=O)n5n4C)c3Cl)c2Cl)ccc1CNC[C@@H]1CCC(=O)N1]

To a mixture of (S)-tert-butyl ((2-(3′-(5-(((tert-butoxycarbonyl)((5-oxopyrrolidin-2-yl)methyl)amino)methyl)-6-methoxypyridin-2-yl)-2,2′-dichloro-[1,1′-biphenyl]-3-yl)-1-methyl-7-oxo-1,7-dihydropyrazolo[1,5-a]pyrimidin-5-yl)methyl)(2-((tert-butyldimethylsilyl)oxy)ethyl)carbamate (0.31 g, 0.31 mmol) in CH₂Cl₂ (1 mL) was added trifluoroacetic acid (3 mL) in one portion under N₂ atmosphere. The mixture was stirred at r.t. for 2 h. The mixture was concentrated. The residue was purified by reversed phase HPLC to afford (S)-2-(2,2′-dichloro-3′-(6-methoxy-5-((((5-oxopyrrolidin-2-yl)methyl)amino)methyl)pyridin-2-yl)-[1,1′-biphenyl]-3-yl)-5-(((2-hydroxyethyl)amino)methyl)-1-methylpyrazolo[1,5-a]pyrimidin-7(1H)-one as a yellow solid as formic salt (19.5 mg, 8.49% yield). MS: m/z found 676 [M+H]⁺; ^{l}H NMR (400 MHz, DMSO-d₆): δ 7.78 (d, J=7.6 Hz, 1H), 7.68-7.63 (m, 5H), 7.52 (t, J=7.6 Hz, 1H), 7.44 (dd, J=7.6 Hz, J=1.6 Hz, 1H), 7.23 (d, J=7.2 Hz, 1H), 6.72 (s, 1H), 6.04 (s, 1H), 3.93 (s, 3H), 3.87 (s, 3H), 3.82 (s, 2H), 3.73-3.64 (m, 2H), 3.61-3.58 (m, 1H), 3.53 (t, J=5.6 Hz, 2H), 2.78 (t, J=5.2 Hz, 2H), 2.53-2.50 (m, 2H), 2.12-2.02 (m, 3H), 1.67-1.61 (m, 1H). ^{l}H NMR (400 MHz, Methanol-d₄): δ 7.68 (d, J=7.2 Hz, 1H), 7.67-7.49 (m, 4H), 7.39 (t, J=7.6 Hz, 1H), 7.44 (dd, J=7.2 Hz, J=1.6 Hz, 1H), 7.15 (d, J=7.2 Hz, 1H), 6.53 (s, 1H), 5.96 (s, 1H), 4.07 (s, 3H), 4.01-3.91 (m, 8H), 3.82-3.79 (m, 1H), 3.70 (t, J=5.2 Hz, 2H), 3.06 (t, J=5.2 Hz, 2H), 2.89-2.80 (m, 2H), 2.29-2.15 (m, 3H), 1.77-1.68 (m, 1H).

### Example 517: (S)-2-(2,2′-Dichloro-3′-(6-methoxy-5-((((5-oxopyrrolidin-2-yl)methyl)amino)methyl)pyridin-2-yl)-[1,1′-biphenyl]-3-yl)-5-((6-hydroxy-2-azaspiro[3.3]heptan-2-yl)methyl)-1-methylpyrazolo[1,5-a]pyrimidin-7(1H)-one

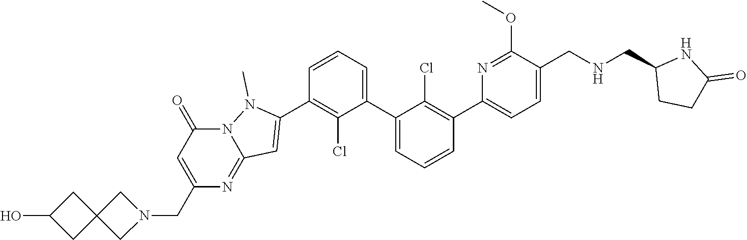 [SMILES: COc1nc(-c2cccc(-c3cccc(-c4cc5nc(CN6CC7(CC(O)C7)C6)cc(=O)n5n4C)c3Cl)c2Cl)ccc1CNC[C@@H]1CCC(=O)N1]

### 2-Bromo-5-((6-hydroxy-2-azaspiro[3.3]heptan-2-yl)methyl)-1-methylpyrazolo[1,5-a]pyrimidin-7(1H)-one

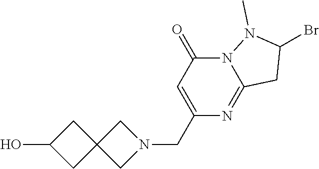 [SMILES: CN1C(Br)Cc2nc(CN3CC4(CC(O)C4)C3)cc(=O)n21]

To a mixture of 2-bromo-5-(chloromethyl)-1-methylpyrazolo[1,5-a]pyrimidin-7(1H)-one (0.3 g, 1.08 mmol) and 2-azaspiro[3.3]heptan-6-ol as a hydrochloride salt (0.18 g, 1.19 mmol) in DMF (4 mL) was added potassium carbonate (0.45 g, 3.25 mmol) in one portion under N₂ atmosphere. The mixture was stirred at 30° C. for 12 h. The mixture was concentrated. The residue was purified by normal phase SiO₂ chromatography (0-80% MeOH/ethyl acetate) to afford 2-bromo-5-((6-hydroxy-2-azaspiro[3.3]heptan-2-yl)methyl)-1-methylpyrazolo[1,5-a]pyrimidin-7(1H)-one as a yellow solid (0.3 g, 48% yield). MS: m/z found 353 [M+H]⁺.

### (S)-tert-Butyl ((6-(2,2′-dichloro-3′-(5-((6-hydroxy-2-azaspiro[3.3]heptan-2-yl)methyl)-1-methyl-7-oxo-1,7-dihydropyrazolo[1,5-a]pyrimidin-2-yl)-[1,1′-biphenyl]-3-yl)-2-methoxypyridin-3-yl)methyl)((5-oxopyrrolidin-2-yl)methyl)carbamate

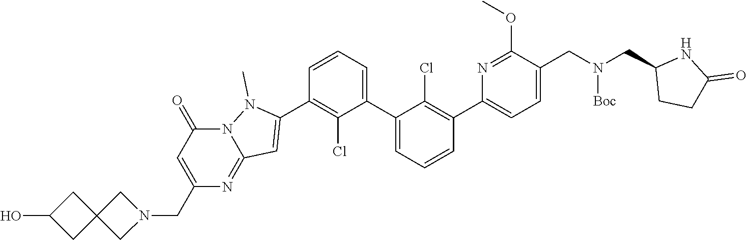 [SMILES: COc1nc(-c2cccc(-c3cccc(-c4cc5nc(CN6CC7(CC(O)C7)C6)cc(=O)n5n4C)c3Cl)c2Cl)ccc1CN(C[C@@H]1CCC(=O)N1)C(=O)OC(C)(C)C]

To a mixture of 2-bromo-5-((6-hydroxy-2-azaspiro[3.3]heptan-2-yl)methyl)-1-methylpyrazolo[1,5-a]pyrimidin-7(1H)-one (0.26 g, 0.73 mmol) and (S)-tert-butyl ((6-(2,2′-dichloro-3′-(4,4,5,5-tetramethyl-1,3,2-dioxaborolan-2-yl)-[1,1′-biphenyl]-3-yl)-2-methoxypyridin-3-yl)methyl)((5-oxopyrrolidin-2-yl)methyl)carbamate (0.25 g, 0.37 mmol) in dioxane/H₂O mixture (8:1, 4.5 mL) was added 1,1′-bis(diphenylphosphino)ferrocene-palladium(ii) dichloride dichloromethane complex (0.015 g, 0.018 mmol) and potassium carbonate (0.15 g, 1.1 mmol) in one portion under N₂ atmosphere. The mixture was stirred at 110° C. for 2 h. The mixture was concentrated. The residue was purified by prep-TLC (silica gel, MeOH/ethyl acetate=8/1) to afford (S)-tert-butyl ((6-(2,2′-dichloro-3′-(5-((6-hydroxy-2-azaspiro[3.3]heptan-2-yl)methyl)-1-methyl-7-oxo-1,7-dihydropyrazolo[1,5-a]pyrimidin-2-yl)-[1,1′-biphenyl]-3-yl)-2-methoxypyridin-3-yl)methyl)((5-oxopyrrolidin-2-yl)methyl)carbamate as a yellow solid (0.15 g, 16% yield). MS: m/z found 828 [M+H]⁺.

### (S)-2-(2,2′-Dichloro-3′-(6-methoxy-5-((((5-oxopyrrolidin-2-yl)methyl)amino)methyl)pyridin-2-yl)-[1,1′-biphenyl]-3-yl)-5-((6-hydroxy-2-azaspiro[3.3]heptan-2-yl)methyl)-1-methylpyrazolo[1,5-a]pyrimidin-7(1H)-one

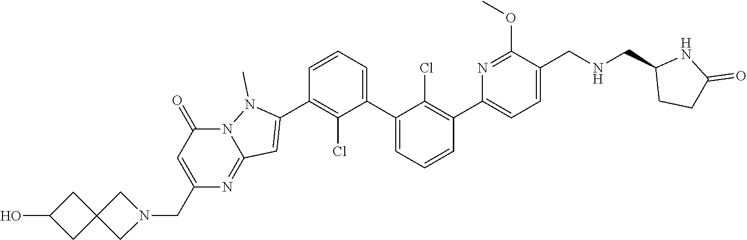 [SMILES: COc1nc(-c2cccc(-c3cccc(-c4cc5nc(CN6CC7(CC(O)C7)C6)cc(=O)n5n4C)c3Cl)c2Cl)ccc1CNC[C@@H]1CCC(=O)N1]

To a mixture of (S)-tert-butyl ((6-(2,2′-dichloro-3′-(5-((6-hydroxy-2-azaspiro[3.3]heptan-2-yl)methyl)-1-methyl-7-oxo-1,7-dihydropyrazolo[1,5-a]pyrimidin-2-yl)-[1,1′-biphenyl]-3-yl)-2-methoxypyridin-3-yl)methyl)((5-oxopyrrolidin-2-yl)methyl)carbamate (0.145 g, 0.17 mmol) in CH₂Cl₂ (0.3 mL) was added trifluoroacetic acid (1.5 mL) in one portion. The mixture was stirred at r.t. for 10 min. The mixture was concentrated. The residue was purified by reversed phase HPLC to afford (S)-2-(2,2′-dichloro-3′-(6-methoxy-5-((((5-oxopyrrolidin-2-yl)methyl)amino)methyl)pyridin-2-yl)-[1,1′-biphenyl]-3-yl)-5-((6-hydroxy-2-azaspiro[3.3]heptan-2-yl)methyl)-1-methylpyrazolo[1,5-a]pyrimidin-7(1H)-one as a light yellow solid (11.7 mg, 8% yield). MS: m/z found 728 [M+H]⁺; ¹H NMR (400 MHz, DMSO-d₆): δ7.84 (d, J=7.2 Hz, 1H), 7.73-7.69 (m, 5H), 7.58 (t, J=7.6 Hz, 1H), 7.50 (dd, J=7.6 Hz, J=1.6 Hz, 1H), 7.29 (d, J=7.2 Hz, 1H), 6.71 (s, 1H), 5.96 (s, 1H), 4.97 (d, J=6.4 Hz, 1H), 3.99-3.93 (m, 7H), 3.79-3.71 (m, 2H), 3.67-3.59 (m, 3H), 3.40-3.33 (m, 4H), 2.58 (d, J=6 Hz, 2H), 2.41-2.36 (m, 2H), 2.16-2.09 (m, 3H), 1.94-1.89 (m, 2H), 1.74-1.69 (m, 1H). ¹H NMR (400 MHz, Methanol-d₄): δ 7.76 (d, J=7.6 Hz, 1H), 7.71-7.65 (m, 4H), 7.54 (t, J=7.6 Hz, 1H), 7.43 (dd, J=7.6 Hz, J=1.6 Hz, 1H), 7.26 (d, J=7.6 Hz, 1H), 6.59 (s, 1H), 6.08 (s, 1H), 4.15-4.08 (m, 4H), 4.04 (s, 3H), 3.90-3.82 (m, 3H), 3.76 (s, 2H), 3.58 (d, J=15.2 Hz, 4H), 2.74-2.66 (m, 2H), 2.56-2.52 (m, 2H), 2.38-2.31 (m, 3H), 2.10-2.05 (m, 2H), 1.85-1.83 (m, 1H).

### Example 513: (3S)-1-[[6-[2-Chloro-3-[2-chloro-3-[6-methoxy-5-[[[(2S)-5-oxopyrrolidin-2-yl]methylamino]methyl]-2-pyridyl]phenyl]phenyl]-3-methyl-4-oxo-pyrrolo[2,1-f][1,2,4]triazin-2-yl]methyl]piperidine-3-carboxylic acid

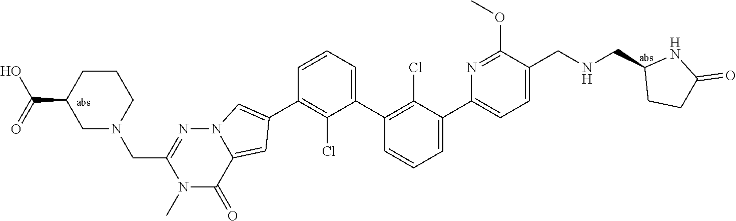 [SMILES: COc1nc(-c2cccc(-c3cccc(-c4cc5c(=O)n(C)c(CN6CCC[C@H](C(=O)O)C6)nn5c4)c3Cl)c2Cl)ccc1CNC[C@@H]1CCC(=O)N1]

### (3S)-1-[[6-[3-[3-[5-[[tert-Butoxycarbonyl-[[(2S)-5-oxopyrrolidin-2-yl]methyl]amino]methyl]-6-methoxy-2-pyridyl]-2-chloro-phenyl]-2-chloro-phenyl]-3-methyl-4-oxo-pyrrolo[2,1-f][1,2,4]triazin-2-yl]methyl]piperidine-3-carboxylic acid

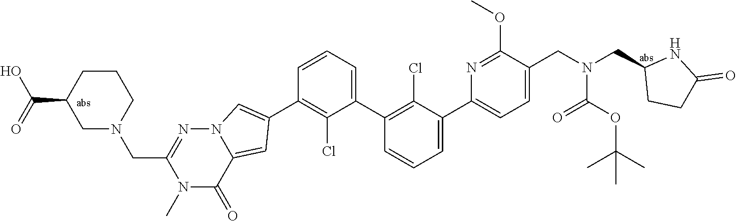 [SMILES: COc1nc(-c2cccc(-c3cccc(-c4cc5c(=O)n(C)c(CN6CCC[C@H](C(=O)O)C6)nn5c4)c3Cl)c2Cl)ccc1CN(C[C@@H]1CCC(=O)N1)C(=O)OC(C)(C)C]

A mixture of tert-butyl N-[[6-[2-chloro-3-[2-chloro-3-(2-formyl-3-methyl-4-oxo-pyrrolo[2,1-f1][1,2,4]triazin-6-yl)phenyl]phenyl]-2-methoxy-3-pyridyl]methyl]-N-[[(2S)-5-oxopyrrolidin-2-yl]methyl]carbamate (10.00 mg, 0.01 mmol), (3S)-piperidine-3-carboxylic acid (3.53 mg, 0.03 mmol) and acetic acid (0.78 uL, 0.82 mg, 0.01 mmol) in MeOH/THF (1:1) was stirred for 1.5 hour at rt. NaBH₃CN (1.72 mg, 0.03 mmol) was added, the mixture was stirred for 30 min, quenched with water and directly purified by reversed phase chromatography to give (3S)-1-[[6-[3-[3-[5-[[tert-butoxycarbonyl-[[(2S)-5-oxopyrrolidin-2-yl]methyl]amino]methyl]-6-methoxy-2-pyridyl]-2-chloro-phenyl]-2-chloro-phenyl]-3-methyl-4-oxo-pyrrolo[2,1-f][1,2,4]triazin-2-yl]methyl]piperidine-3-carboxylic acid (4.00 mg, 34.6%). MS: m/z found 844.3 [M+H]⁺.

### (3S)-1-[[6-[2-Chloro-3-[2-chloro-3-[6-methoxy-5-[[[(2S)-5-oxopyrrolidin-2-yl]methylamino]methyl]-2-pyridyl]phenyl]phenyl]-3-methyl-4-oxo-pyrrolo[2,1-f][1,2,4]triazin-2-yl]methyl]piperidine-3-carboxylic acid

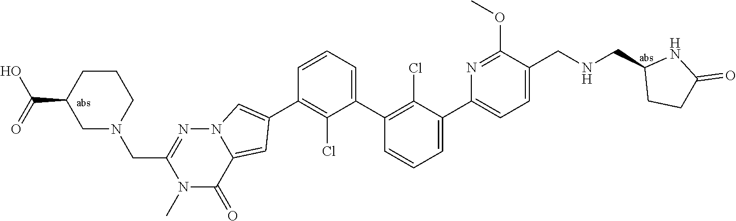 [SMILES: COc1nc(-c2cccc(-c3cccc(-c4cc5c(=O)n(C)c(CN6CCC[C@H](C(=O)O)C6)nn5c4)c3Cl)c2Cl)ccc1CNC[C@@H]1CCC(=O)N1]

A solution of (3S)-1-[[6-[3-[3-[5-[[tert-butoxycarbonyl-[[(2S)-5-oxopyrrolidin-2-yl]methyl]amino]methyl]-6-methoxy-2-pyridyl]-2-chloro-phenyl]-2-chloro-phenyl]-3-methyl-4-oxo-pyrrolo[2,1-f][1,2,4]triazin-2-yl]methyl]piperidine-3-carboxylic acid (7.00 mg, 0.01 mmol) in DCM/TFA (2:1) was stirred for 30 min, concentrated and purified by reversed phase chromatography to give (3S)-1-[[6-[2-chloro-3-[2-chloro-3-[6-methoxy-5-[[[(2S)-5-oxopyrrolidin-2-yl]methylamino]methyl]-2-pyridyl]phenyl]phenyl]-3-methyl-4-oxo-pyrrolo[2,1-f][1,2,4]triazin-2-yl]methyl]piperidine-3-carboxylic acid (6.00 mg, 97.2%). MS: m/z found 744.2 [M+H]⁺; ¹H NMR (400 MHz, Methanol-d₄): δ 7.83 (d, J=1.9 Hz, 1H), 7.77 (d, J=7.5 Hz, 1H), 7.63 (dt, J=7.7, 1.8 Hz, 2H), 7.49 (t, J=7.7 Hz, 1H), 7.44 (t, J=7.7 Hz, 1H), 7.36 (dd, J=7.6, 1.7 Hz, 1H), 7.30-7.24 (m, 3H), 4.04 (s, 3H), 3.99-3.92 (m, 2H), 3.92-3.86 (m, 1H), 3.66 (s, 3H), 3.62 (s, 2H), 3.05-2.92 (m, 1H), 2.89-2.71 (m, 3H), 2.54 (s, 2H), 2.42-2.24 (m, 4H), 1.93-1.73 (m, 3H), 1.69-1.52 (m, 2H).

### Example 516: 3-[[6-[2-Chloro-3-[2-chloro-3-[6-methoxy-5-[[[(2S)-5-oxopyrrolidin-2-yl]methylamino]methyl]-2-pyridyl]phenyl]phenyl]-3-methyl-4-oxo-pyrrolo[2,1-f][1,2,4]triazin-2-yl]methylamino]propanoic acid

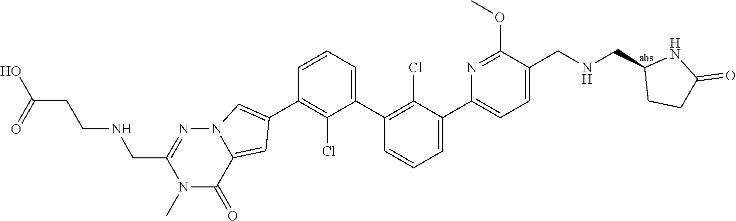 [SMILES: COc1nc(-c2cccc(-c3cccc(-c4cc5c(=O)n(C)c(CNCCC(=O)O)nn5c4)c3Cl)c2Cl)ccc1CNC[C@@H]1CCC(=O)N1]

### 3-[[6-[3-[3-[5-[[tert-butoxycarbonyl-[[(2S)-5-oxopyrrolidin-2-yl]methyl]amino]methyl]-6-methoxy-2-pyridyl]-2-chloro-phenyl]-2-chloro-phenyl]-3-methyl-4-oxo-pyrrolo[2,1-f][1,2,4]triazin-2-yl]methylamino]propanoic acid

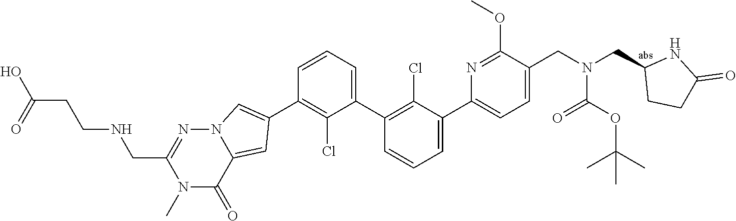 [SMILES: COc1nc(-c2cccc(-c3cccc(-c4cc5c(=O)n(C)c(CNCCC(=O)O)nn5c4)c3Cl)c2Cl)ccc1CN(C[C@@H]1CCC(=O)N1)C(=O)OC(C)(C)C]

A mixture of tert-butyl N-[[6-[2-chloro-3-[2-chloro-3-(2-formyl-3-methyl-4-oxo-pyrrolo[2,1-f1][1,2,4]triazin-6-yl)phenyl]phenyl]-2-methoxy-3-pyridyl]methyl]-N-[[(2S)-5-oxopyrrolidin-2-yl]methyl]carbamate (15.00 mg, 0.02 mmol), 3-aminopropanoic acid (3.65 mg, 0.04 mmol) and acetic acid (1.17 uL, 1.23 mg, 0.02 mmol) in MeOH/THF (1:1) was stirred for 4 hours at rt. NaBH₃CN (2.58 mg, 0.04 mmol) was added, the mixture was stirred for 30 min, quenched with water and directly purified by reversed phase chromatography to give 3-[[6-[3-[3-[5-[[tert-butoxycarbonyl-[[(2S)-5-oxopyrrolidin-2-yl]methyl]amino]methyl]-6-methoxy-2-pyridyl]-2-chloro-phenyl]-2-chloro-phenyl]-3-methyl-4-oxo-pyrrolo[2,1-f][1,2,4]triazin-2-yl]methylamino]propanoic acid (6.00 mg, 36.4%). MS: m/z found 804.3 [M+H]⁺.

### 3-[[6-[2-Chloro-3-[2-chloro-3-[6-methoxy-5-[[[(2S)-5-oxopyrrolidin-2-yl]methylamino]methyl]-2-pyridyl]phenyl]phenyl]-3-methyl-4-oxo-pyrrolo[2,1-f][1,2,4]triazin-2-yl]methylamino]propanoic acid

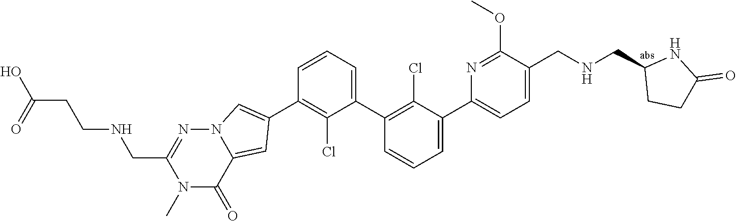 [SMILES: COc1nc(-c2cccc(-c3cccc(-c4cc5c(=O)n(C)c(CNCCC(=O)O)nn5c4)c3Cl)c2Cl)ccc1CNC[C@@H]1CCC(=O)N1]

A solution of 3-[[6-[3-[3-[5-[[tert-butoxycarbonyl-[[(2S)-5-oxopyrrolidin-2-yl]methyl]amino]methyl]-6-methoxy-2-pyridyl]-2-chloro-phenyl]-2-chloro-phenyl]-3-methyl-4-oxo-pyrrolo[2,1-f][1,2,4]triazin-2-yl]methylamino]propanoic acid (5.00 mg, 0.01 mmol) in DCM/TFA (2:1) was stirred for 30 min, concentrated and purified by reversed phase chromatography to give 3-[[6-[2-chloro-3-[2-chloro-3-[6-methoxy-5-[[[(2S)-5-oxopyrrolidin-2-yl]methylamino]methyl]-2-pyridyl]phenyl]phenyl]-3-methyl-4-oxo-pyrrolo[2,1-f][1,2,4]triazin-2-yl]methylamino]propanoic acid (4.25 mg, 97.10%). MS: m/z found 704.2 [M+H]⁺; ¹H NMR (400 MHz, Methanol-d₄): δ 7.93 (d, J=1.8 Hz, 1H), 7.77-7.71 (m, 1H), 7.64 (m, 2H), 7.49 (t, J=7.3 Hz, 1H), 7.47-7.43 (m, 1H), 7.39-7.33 (m, 1H), 7.32-7.22 (m, 3H), 4.03 (s, 3H), 4.02 (s, 2H), 3.89-3.85 (m, 3H), 3.53 (s, 3H), 3.24 (t, J=6.3 Hz, 2H), 2.75 (t, J=6.2 Hz, 2H), 2.58 (t, J=6.5 Hz, 2H), 2.38-2.24 (m, 3H), 1.88-1.76 (m, 1H).

### Example 515: (5S)-5-[[[6-[3-[3-[2-[[(1-Acetyl-4-piperidyl)amino]methyl]-[1,2,4]triazolo[1,5-a]pyrimidin-6-yl]-2-chloro-phenyl]-2-chloro-phenyl]-2-methoxy-3-pyridyl]methylamino]methyl] pyrrolidin-2-one

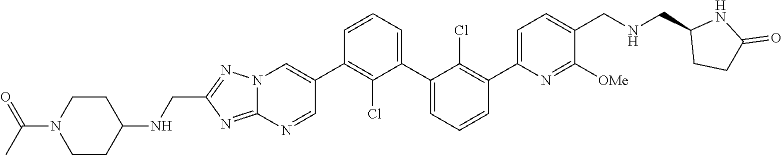 [SMILES: COc1nc(-c2cccc(-c3cccc(-c4cnc5nc(CNC6CCN(C(C)=O)CC6)nn5c4)c3Cl)c2Cl)ccc1CNC[C@@H]1CCC(=O)N1]

### tert-Butyl N-[[6-[3-[3-[2-[[(1-acetyl-4-piperidyl)amino]methyl]-[1,2,4]triazolo[1,5-a]pyrimidin-6-yl]-2-chloro-phenyl]-2-chloro-phenyl]-2-methoxy-3-pyridyl]methyl]-N-[[(2S)-5-oxopyrrolidin-2-yl]methyl]carbamate

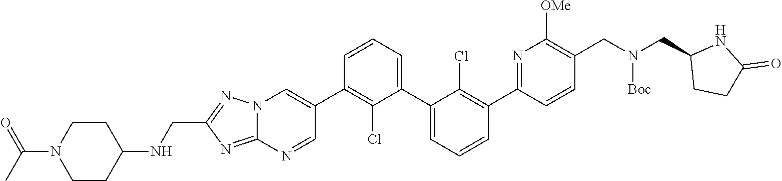 [SMILES: COc1nc(-c2cccc(-c3cccc(-c4cnc5nc(CNC6CCN(C(C)=O)CC6)nn5c4)c3Cl)c2Cl)ccc1CN(C[C@@H]1CCC(=O)N1)C(=O)OC(C)(C)C]

To a solution of tert-butyl N-[[6-[2-chloro-3-[2-chloro-3-[2-(hydroxymethyl)-[1,2,4]triazolo[1,5-a]pyrimidin-6-yl]phenyl]phenyl]-2-methoxy-3-pyridyl]methyl]-N-[[(2S)-5-oxopyrrolidin-2-yl]methyl]carbamate (23 mg, 0.03 mmol) in DCM (0.4 mL) was added mesyl chloride (3.8 uL, 0.05 mmol) and triethylamine (13 uL, 0.1 mmol). The reaction was stirred at rt for 1 h. The solvent was evaporated, and the residue was taken up in DMF (0.4 mL). Potassium carbonate (13.53 mg, 0.10 mmol) and 1-(4-amino-1-piperidyl)ethanone (11.18 mg, 0.10 mmol) were added and the reaction was stirred at rt for 12 h. The solvent was evaporated, and the residue was suspended in water. The precipitate formed was collected to afford tert-butyl N-[[6-[3-[3-[2-[[(1-acetyl-4-piperidyl)amino]methyl]-[1,2,4]triazolo[1,5-a]pyrimidin-6-yl]-2-chloro-phenyl]-2-chloro-phenyl]-2-methoxy-3-pyridyl]methyl]-N-[[(2S)-5-oxopyrrolidin-2-yl]methyl]carbamate (24 mg, 89%). The material was used as is without any purification. m/z: 828.2 [M+H]⁺ observed.

### (5S)-5-[[[6-[3-[3-[2-[[(1-Acetyl-4-piperidyl)amino]methyl]-[1,2,4]triazolo[1,5-a]pyrimidin-6-yl]-2-chloro-phenyl]-2-chloro-phenyl]-2-methoxy-3-pyridyl]methylamino]methyl] pyrrolidin-2-one

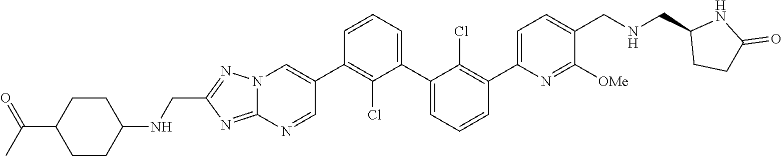 [SMILES: COc1nc(-c2cccc(-c3cccc(-c4cnc5nc(CNC6CCC(C(C)=O)CC6)nn5c4)c3Cl)c2Cl)ccc1CNC[C@@H]1CCC(=O)N1]

To a solution of tert-butyl N-[[6-[3-[3-[2-[[(1-acetyl-4-piperidyl)amino]methyl]-[1,2,4]triazolo[1,5-a]pyrimidin-6-yl]-2-chloro-phenyl]-2-chloro-phenyl]-2-methoxy-3-pyridyl]methyl]-N-[[(2S)-5-oxopyrrolidin-2-yl]methyl]carbamate (24 mg, 0.03 mmol) in DCM (0.1 mL) was added TFA (44 uL, 0.58 mmol). The reaction was stirred at rt for 1 h.

The solvent was evaporated and the residue was purified by reverse phase HPLC (acetonitrile:water) to afford (5S)-5-[[[6-[3-[3-[2-[[(1-acetyl-4-piperidyl)amino]methyl]-[1,2,4]triazolo[1,5-a]pyrimidin-6-yl]-2-chloro-phenyl]-2-chloro-phenyl]-2-methoxy-3-pyridyl]methylamino]methyl]pyrrolidin-2-one (11 mg, 52.1%). m/z: 729.0 [M+H]⁺. ¹H NMR (400 MHz, Methanol-d₄) δ 9.40 (s, 1H), 9.03 (d, J=2.3 Hz, 1H), 7.82 (d, J=7.7 Hz, 1H), 7.65 (s, 2H), 7.60 (s, 1H), 7.51 (d, J=7.5 Hz, 2H), 7.41 (d, J=7.7 Hz, 1H), 7.30 (d, J=7.4 Hz, 1H), 5.03 (s, 2H), 4.94 (d, J=5.8 Hz, 2H), 4.34 (s, 2H), 4.10 (s, 3H), 3.20-3.11 (m, 2H), 3.01 (dt, J=12.4, 6.6 Hz, 3H), 2.38 (d, J=16.6 Hz, 4H), 2.11 (s, 4H), 1.50 (t, J=7.1 Hz, 3H).

### HTRF Assay

PD-L1 His protein was prepared and added at the final concentration of 6 nM in the White opaque 384 well plate (Corning cat #3824BC). PD-L1 small molecule inhibitors were diluted by 3-fold starting from 20 μM and a final concentration of 0.001 μM and added to the well. PD-1 Fc protein was added at the final concentration of 6 nM. PD-L1 His protein, PD-L1 small molecule inhibitors, and PD1 Fc proteins were added to the well in this order, with each 5 μl volume, and were incubated for 15 minutes at room temperature. PAb anti-Human IgG-XL665 (Cisbio, cat #61HFCXLA) and Mab anti-6HIS Tb cryptate Gold (Cisbio, cat #61HI2TLA) were mixed at 6.7 nM and 0.35 nM respectively, and total 5 μl volume of mixture was added to the well and incubated at room temperature for 1 hour. The plate was read using PerkinElmer Envision plate reader and data was analyzed by Prism 6 software. Results are illustrated in Table 1.

TABLE 1

| | | PD-L1 |
|---|---|---|
| | | HTRF |
| | | IC₅₀ |
| Ex. | COMPOUND | (uM) |
| 1 | 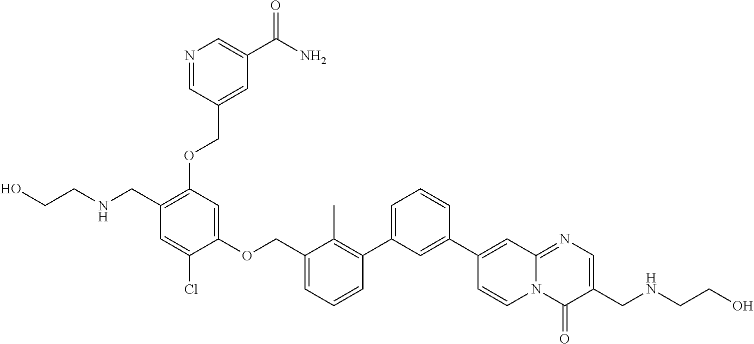 [SMILES: Cc1c(COc2cc(OCc3cncc(C(N)=O)c3)c(CNCCO)cc2Cl)cccc1-c1cccc(-c2ccn3c(=O)c(CNCCO)cnc3c2)c1] | 0.0028 |
| | 5-((4-chloro-2-(((2-hydroxyethyl)amino)methyl)-5-((3′-(3-(((2-hydroxyethyl)amino)methyl)-4-oxo-4H-pyrido[1,2-a]pyrimidin- | |
| | 8-yl)-2-methyl-[1,1′-biphenyl]-3-yl)methoxy)phenoxy)methyl)nicotinamide | |
| 2 | 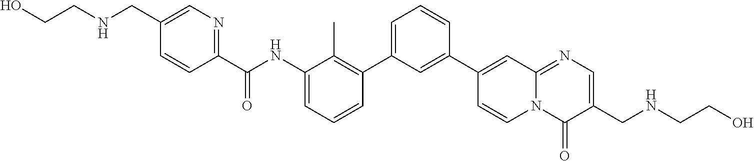 [SMILES: Cc1c(NC(=O)c2ccc(CNCCO)cn2)cccc1-c1cccc(-c2ccn3c(=O)c(CNCCO)cnc3c2)c1] | 0.079 |
| | 5-(((2-hydroxyethyl)amino)methyl)-N-(3′-(3-(((2-hydroxyethyl)amino)methyl)-4-oxo-4H-pyrido[1,2-a]pyrimidin-8-yl)-2- | |
| | methyl-[1,1′-biphenyl]-3-yl)picolinamide | |
| 3 | 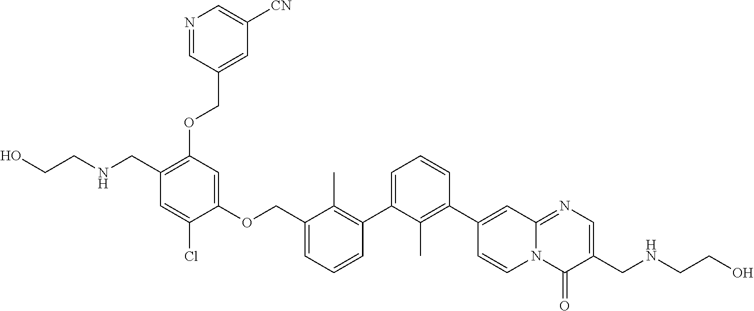 [SMILES: Cc1c(COc2cc(OCc3cncc(C#N)c3)c(CNCCO)cc2Cl)cccc1-c1cccc(-c2ccn3c(=O)c(CNCCO)cnc3c2)c1C] | 0.0012 |
| | 5-((4-chloro-2-(((2-hydroxyethyl)amino)methyl)-5-((3′-(3-(((2-hydroxyethyl)amino)methyl)-4-oxo-4H-pyrido[1,2-a]pyrimidin- | |
| | 8-yl)-2,2′-dimethyl-[1,1′-biphenyl]-3-yl)methoxy)phenoxy)methyl)nicotinonitrile | |
| 4 | 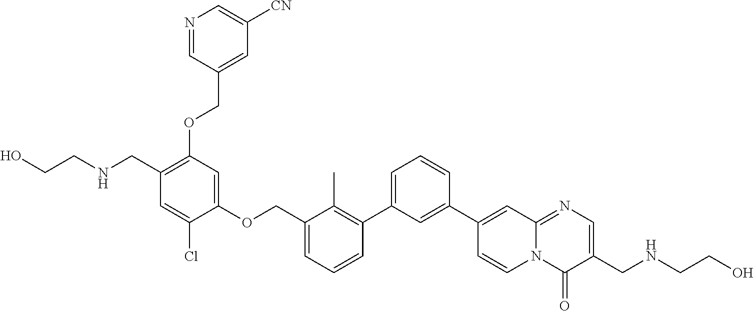 [SMILES: Cc1c(COc2cc(OCc3cncc(C#N)c3)c(CNCCO)cc2Cl)cccc1-c1cccc(-c2ccn3c(=O)c(CNCCO)cnc3c2)c1] | 0.002 |
| | 5-((4-chloro-2-(((2-hydroxyethyl)amino)methyl)-5-((3′-(3-(((2-hydroxyethyl)amino)methyl)-4-oxo-4H-pyrido[1,2-a]pyrimidin- | |
| | 8-yl)-2-methyl-[1,1′-biphenyl]-3-yl)methoxy)phenoxy)methyl)nicotinonitrile | |
| 5 | 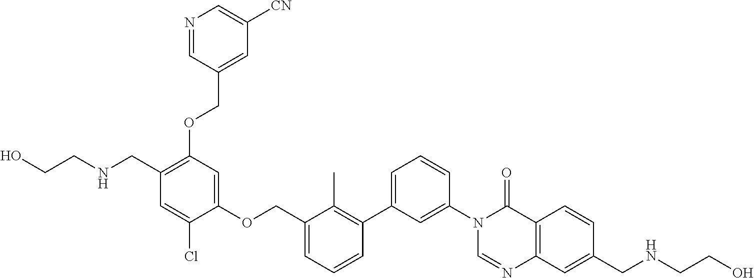 [SMILES: Cc1c(COc2cc(OCc3cncc(C#N)c3)c(CNCCO)cc2Cl)cccc1-c1cccc(-n2cnc3cc(CNCCO)ccc3c2=O)c1] | 0.15 |
| | 5-((4-chloro-2-(((2-hydroxyethyl)amino)methyl)-5-((3′-(7-(((2-hydroxyethyl)amino)methyl)-4-oxoquinazolin-3(4H)-yl)-2- | |
| | methyl-[1,1′-biphenyl]-3-yl)methoxy)phenoxy)methyl)nicotinonitrile | |
| 6 | 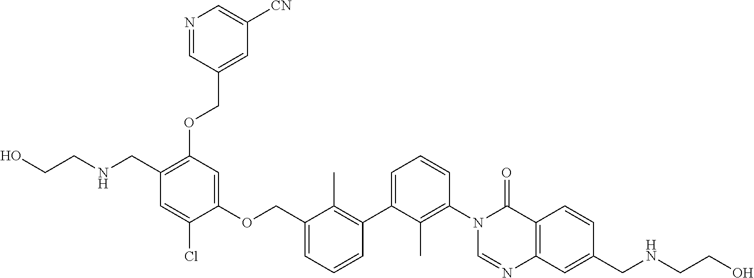 [SMILES: Cc1c(COc2cc(OCc3cncc(C#N)c3)c(CNCCO)cc2Cl)cccc1-c1cccc(-n2cnc3cc(CNCCO)ccc3c2=O)c1C] | 0.044 |
| | 5-((4-chloro-2-(((2-hydroxyethyl)amino)methyl)-5-((3′-(7-(((2-hydroxyethyl)amino)methyl)-4-oxoquinazolin-3(4H)-yl)-2,2′- | |
| | dimethyl-[1,1′-biphenyl]-3-yl)methoxy)phenoxy)methyl)nicotinonitrile | |
| 7 | 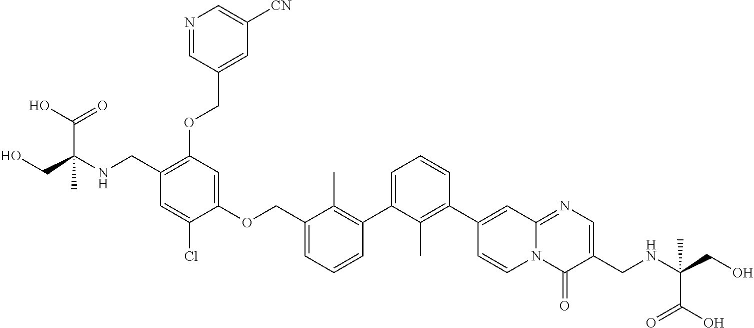 [SMILES: Cc1c(COc2cc(OCc3cncc(C#N)c3)c(CN[C@@](C)(CO)C(=O)O)cc2Cl)cccc1-c1cccc(-c2ccn3c(=O)c(CN[C@@](C)(CO)C(=O)O)cnc3c2)c1C] | 0.0002 |
| | (S)-2-(((8-(3′-((4-((((S)-2-carboxy-1-hydroxypropan-2-yl)amino)methyl)-2-chloro-5-((5-cyanopyridin-3- | |
| | yl)methoxy)phenoxy)methyl)-2,2′-dimethyl-[1,1′-biphenyl]-3-yl)-4-oxo-4H-pyrido[1,2-a]pyrimidin-3-yl)methyl)amino)-3- | |
| | hydroxy-2-methylpropanoic acid | |
| 8 | 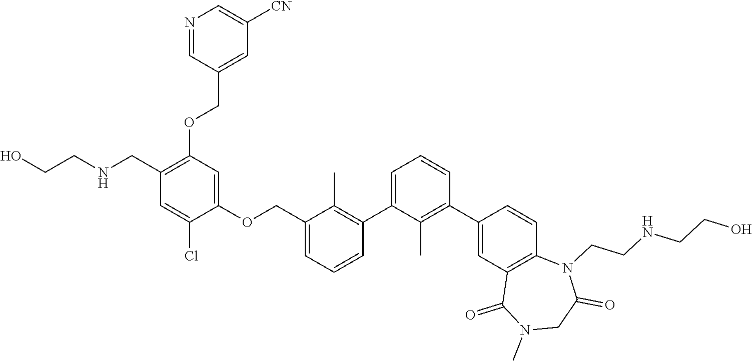 [SMILES: Cc1c(COc2cc(OCc3cncc(C#N)c3)c(CNCCO)cc2Cl)cccc1-c1cccc(-c2ccc3c(c2)C(=O)N(C)CC(=O)N3CCNCCO)c1C] | 0.0008 |
| | 5-((4-chloro-5-((3′-(1-(2-((2-hydroxyethyl)amino)ethyl)-4-methyl-2,5-dioxo-2,3,4,5-tetrahydro-1H-benzo[e][1,4]diazepin-7-yl)- | |
| | 2,2′-dimethyl-[1,1′-biphenyl]-3-yl)methoxy)-2-(((2-hydroxyethyl)amino)methyl)phenoxy)methyl)nicotinonitrile | |
| 9 | 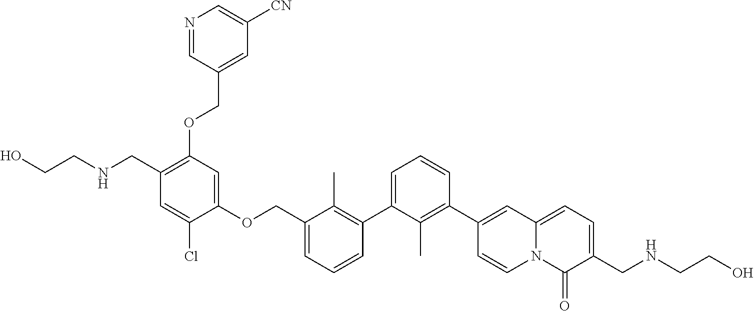 [SMILES: Cc1c(COc2cc(OCc3cncc(C#N)c3)c(CNCCO)cc2Cl)cccc1-c1cccc(-c2ccn3c(=O)c(CNCCO)ccc3c2)c1C] | 0.0011 |
| | 5-((4-chloro-2-(((2-hydroxyethyl)amino)methyl)-5-((3′-(3-(((2-hydroxyethyl)amino)methyl)-4-oxo-4H-quinolizin-8-yl)-2,2′- | |
| | dimethyl-[1,1′-biphenyl]-3-yl)methoxy)phenoxy)methyl)nicotinonitrile | |
| 10 | 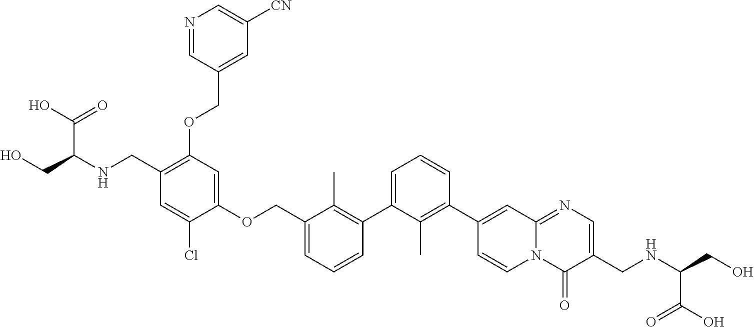 [SMILES: Cc1c(COc2cc(OCc3cncc(C#N)c3)c(CN[C@@H](CO)C(=O)O)cc2Cl)cccc1-c1cccc(-c2ccn3c(=O)c(CN[C@@H](CO)C(=O)O)cnc3c2)c1C] | 0.0003 |
| | ((8-(3′-((4-((((S)-1-carboxy-2-hydroxyethyl)amino)methyl)-2-chloro-5-((5-cyanopyridin-3-yl)methoxy)phenoxy)methyl)-2,2′- | |
| | dimethyl-[1,1′-biphenyl]-3-yl)-4-oxo-4H-pyrido[1,2-a]pyrimidin-3-yl)methyl)-L-serine or (S)-2-(((8-(3′-((4-((((S)-2-carboxy-1- | |
| | hydroxypropan-2-yl)amino)methyl)-2-chloro-5-((5-cyanopyridin-3-yl)methoxy)phenoxy)methyl)-2,2′-dimethyl-[1,1′-biphenyl]- | |
| | 3-yl)-4-oxo-4H-pyrido[1,2-a]pyrimidin-3-yl)methyl)amino)-3-hydroxy-2-methylpropanoic acid | |
| 11 | 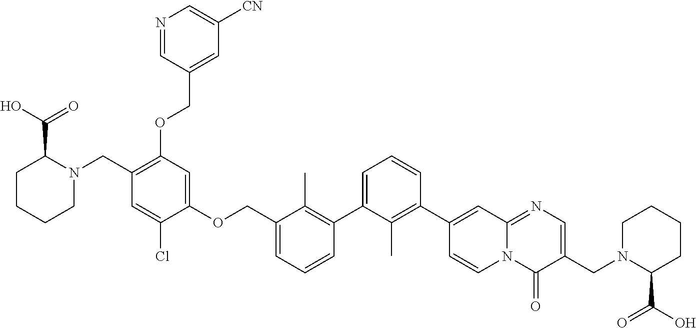 [SMILES: Cc1c(COc2cc(OCc3cncc(C#N)c3)c(CN3CCCC[C@H]3C(=O)O)cc2Cl)cccc1-c1cccc(-c2ccn3c(=O)c(CN4CCCC[C@H]4C(=O)O)cnc3c2)c1C] | 0.0006 |
| | (S)-1-((8-(3′-((4-(((S)-2-carboxypiperidin-1-yl)methyl)-2-chloro-5-((5-cyanopyridin-3-yl)methoxy)phenoxy)methyl)- | |
| | 2,2′-dimethyl-[1,1′-biphenyl]-3-yl)-4-oxo-4H-pyrido[1,2-a]pyrimidin-3-yl)methyl)piperidine-2-carboxylic acid | |
| 12 | 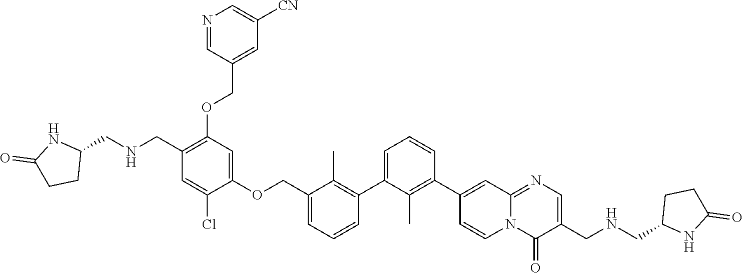 [SMILES: Cc1c(COc2cc(OCc3cncc(C#N)c3)c(CNC[C@@H]3CCC(=O)N3)cc2Cl)cccc1-c1cccc(-c2ccn3c(=O)c(CNC[C@@H]4CCC(=O)N4)cnc3c2)c1C] | 0.0003 |
| | 5-((4-chloro-5-((2,2′-dimethyl-3′-(4-oxo-3-(((((S)-5-oxopyrrolidin-2-yl)methyl)amino)methyl)-4H-pyrido[1,2-a]pyrimidin-8- | |
| | yl)-[1,1′-biphenyl]-3-yl)methoxy)-2-(((((S)-5-oxopyrrolidin-2-yl)methyl)amino)methyl)phenoxy)methyl)nicotinonitrile | |
| 13 | 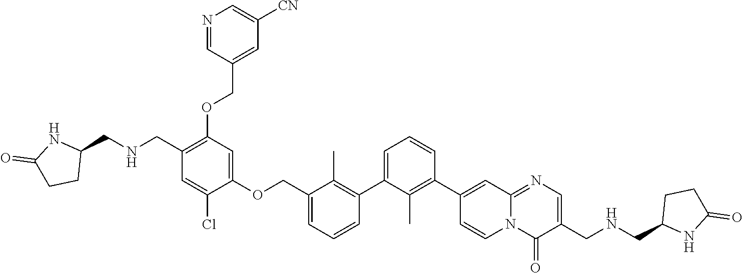 [SMILES: Cc1c(COc2cc(OCc3cncc(C#N)c3)c(CNC[C@H]3CCC(=O)N3)cc2Cl)cccc1-c1cccc(-c2ccn3c(=O)c(CNC[C@H]4CCC(=O)N4)cnc3c2)c1C] | 0.0008 |
| | 5-((4-chloro-5-((2,2′-dimethyl-3′-(4-oxo-3-(((((R)-5-oxopyrrolidin-2-yl)methyl)amino)methyl)-4H-pyrido[1,2-a]pyrimidin-8- | |
| | yl)-[1,1′-biphenyl]-3-yl)methoxy)-2-(((((R)-5-oxopyrrolidin-2-yl)methyl)amino)methyl)phenoxy)methyl)nicotinonitrile | |
| 14 | 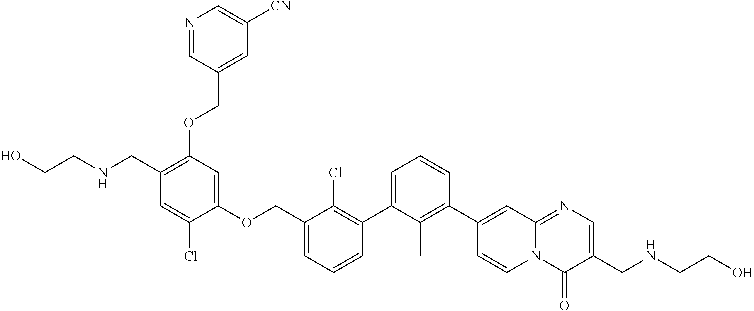 [SMILES: Cc1c(-c2ccn3c(=O)c(CNCCO)cnc3c2)cccc1-c1cccc(COc2cc(OCc3cncc(C#N)c3)c(CNCCO)cc2Cl)c1Cl] | 0.0004 |
| | 5-((4-chloro-5-((2-chloro-3′-(3-(((2-hydroxyethyl)amino)methyl)-4-oxo-4H-pyrido[1,2-a]pyrimidin-8-yl)-2′-methyl-[1,1′- | |
| | biphenyl]-3-yl)methoxy)-2-(((2-hydroxyethyl)amino)methyl)phenoxy)methyl)nicotinonitrile | |
| 15 | 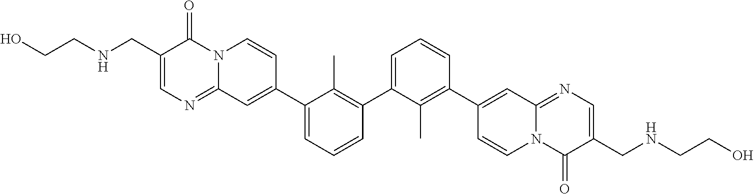 [SMILES: Cc1c(-c2ccn3c(=O)c(CNCCO)cnc3c2)cccc1-c1cccc(-c2ccn3c(=O)c(CNCCO)cnc3c2)c1C] | 0.060 |
| | 8,8′-(2,2′-dimethyl-[1,1′-biphenyl]-3,3′-diyl)bis(3-(((2-hydroxyethyl)amino)methyl)-4H-pyrido[1,2-a]pyrimidin-4-one) | |
| 16 | 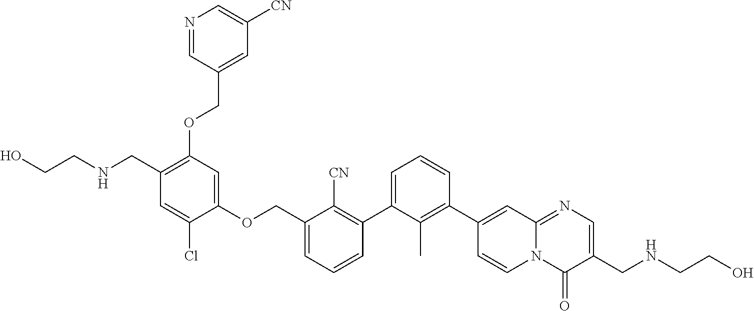 [SMILES: Cc1c(-c2ccn3c(=O)c(CNCCO)cnc3c2)cccc1-c1cccc(COc2cc(OCc3cncc(C#N)c3)c(CNCCO)cc2Cl)c1C#N] | 0.0007 |
| | 5-((4-chloro-5-((2-cyano-3′-(3-(((2-hydroxyethyl)amino)methyl)-4-oxo-4H-pyrido[1,2-a]pyrimidin-8-yl)-2′-methyl-[1,1′- | |
| | biphenyl]-3-yl)methoxy)-2-(((2-hydroxyethyl)amino)methyl)phenoxy)methyl)nicotinonitrile | |
| 17 | 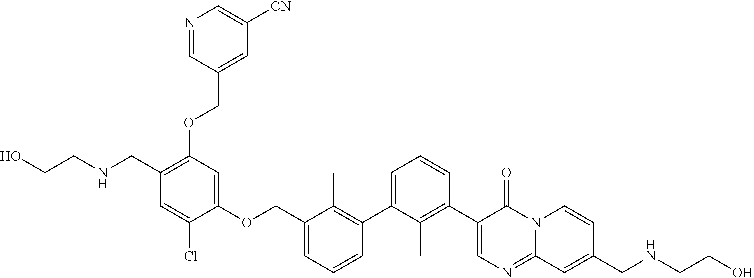 [SMILES: Cc1c(COc2cc(OCc3cncc(C#N)c3)c(CNCCO)cc2Cl)cccc1-c1cccc(-c2cnc3cc(CNCCO)ccn3c2=O)c1C] | 0.002 |
| | 5-((4-chloro-2-(((2-hydroxyethyl)amino)methyl)-5-((3′-(8-(((2-hydroxyethyl)amino)methyl)-4-oxo-4H-pyrido[1,2-a]pyrimidin- | |
| | 3-yl)-2,2′-dimethyl-[1,1′-biphenyl]-3-yl)methoxy)phenoxy)methyl)nicotinonitrile | |
| 18 | 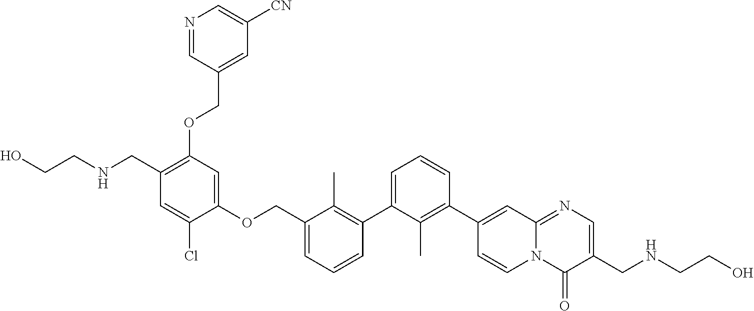 [SMILES: Cc1c(COc2cc(OCc3cncc(C#N)c3)c(CNCCO)cc2Cl)cccc1-c1cccc(-c2ccn3c(=O)c(CNCCO)cnc3c2)c1C] | 0.0048 |
| | 5-((4-chloro-2-(((2-hydroxyethyl)amino)methyl)-5-(((3′-(3-(((2-hydroxyethyl)amino)methyl)-4-oxo-4H-pyrido[1,2-a]pyrimidin- | |
| | 8-yl)-2,2′-dimethyl-[1,1′-biphenyl]-3-yl)oxy)methyl)phenoxy)methyl)nicotinonitrile | |
| 19 | 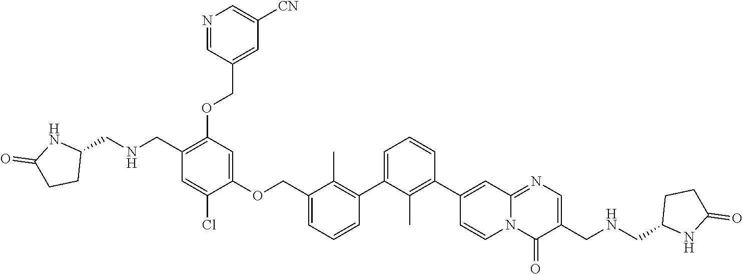 [SMILES: Cc1c(COc2cc(OCc3cncc(C#N)c3)c(CNC[C@@H]3CCC(=O)N3)cc2Cl)cccc1-c1cccc(-c2ccn3c(=O)c(CNC[C@@H]4CCC(=O)N4)cnc3c2)c1C] | 0.013 |
| | 5-((4-chloro-5-(((2,2′-dimethyl-3′-(4-oxo-3-(((((S)-5-oxopyrrolidin-2-yl)methyl)amino)methyl)-4H-pyrido[1,2-a]pyrimidin-8- | |
| | yl)-[1,1′-biphenyl]-3-yl)oxy)methyl)-2-(((((S)-5-oxopyrrolidin-2-yl)methyl)amino)methyl)phenoxy)methyl)nicotinonitrile | |
| 20 | 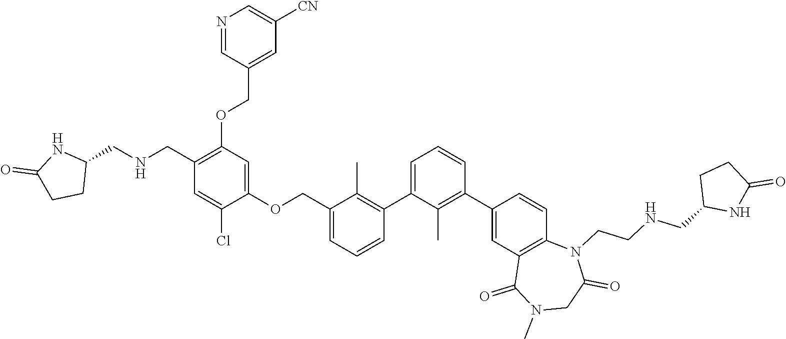 [SMILES: Cc1c(COc2cc(OCc3cncc(C#N)c3)c(CNC[C@@H]3CCC(=O)N3)cc2Cl)cccc1-c1cccc(-c2ccc3c(c2)C(=O)N(C)CC(=O)N3CCNC[C@@H]2CCC(=O)N2)c1C] | 0.0042 |
| | 5-((4-chloro-5-((2,2′-dimethyl-3′-(4-methyl-2,5-dioxo-1-(2-((((S)-5-oxopyrrolidin-2-yl)methyl)amino)ethyl)-2,3,4,5-tetrahydro-1H- | |
| | benzo[e][1,4]diazepin-7-yl)-[1,1′-biphenyl]-3-yl)methoxy)-2-(((((S)-5-oxopyrrolidin-2- | |
| | yl)methyl)amino)methyl)phenoxy)methyl)nicotinonitrile | |
| 21 | 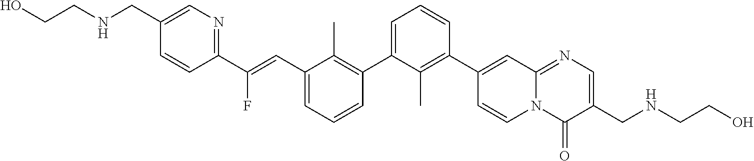 [SMILES: Cc1c(/C=C(\F)c2ccc(CNCCO)cn2)cccc1-c1cccc(-c2ccn3c(=O)c(CNCCO)cnc3c2)c1C] | 0.0048 |
| | (Z)-8-(3′-(2-fluoro-2-(5-(((2-hydroxyethyl)amino)methyl)pyridin-2-yl)vinyl)-2,2′-dimethyl-[1,1′-biphenyl]-3-yl)-3-(((2- | |
| | hydroxyethyl)amino)methyl)-4H-pyrido[1,2-a]pyrimidin-4-one | |
| 22 | 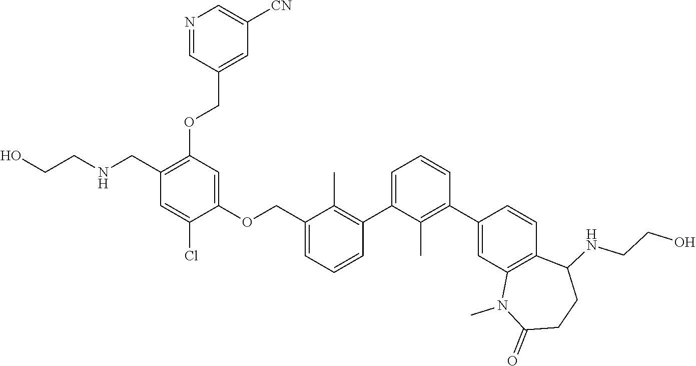 [SMILES: Cc1c(COc2cc(OCc3cncc(C#N)c3)c(CNCCO)cc2Cl)cccc1-c1cccc(-c2ccc3c(c2)N(C)C(=O)CCC3NCCO)c1C] | 0.006 |
| | 5-((4-chloro-5-((3′-(5-((2-hydroxyethyl)amino)-1-methyl-2-oxo-2,3,4,5-tetrahydro-1H-benzo[b]azepin-8-yl)-2,2′- | |
| | dimethyl-[1,1′-biphenyl]-3-yl)methoxy)-2-(((2-hydroxyethyl)amino)methyl)phenoxy)methyl)nicotinonitrile | |
| 23 | 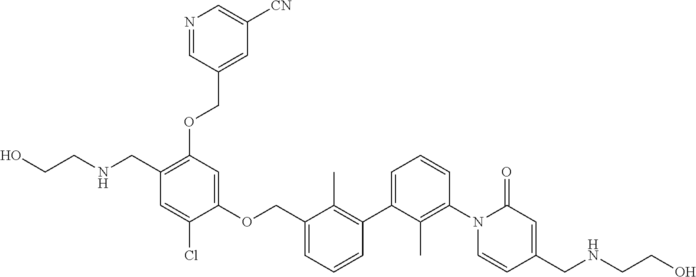 [SMILES: Cc1c(COc2cc(OCc3cncc(C#N)c3)c(CNCCO)cc2Cl)cccc1-c1cccc(-n2ccc(CNCCO)cc2=O)c1C] | 0.033 |
| | 5-((4-chloro-2-(((2-hydroxyethyl)amino)methyl)-5-((3′-(4-(((2-hydroxyethyl)amino)methyl)-2-oxopyridin-1(2H)-yl)- | |
| | 2,2′-dimethyl-[1,1′-biphenyl]-3-yl)methoxy)phenoxy)methyl)nicotinonitrile | |
| 24 | 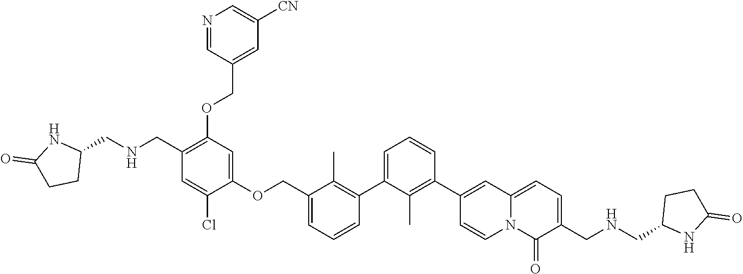 [SMILES: Cc1c(COc2cc(OCc3cncc(C#N)c3)c(CNC[C@@H]3CCC(=O)N3)cc2Cl)cccc1-c1cccc(-c2ccn3c(=O)c(CNC[C@@H]4CCC(=O)N4)ccc3c2)c1C] | 0.0005 |
| | 5-((4-chloro-5-((2,2′-dimethyl-3′-(4-oxo-3-(((((S)-5-oxopyrrolidin-2-yl)methyl)amino)methyl)-4H-quinolizin-8-yl)-[1,1′- | |
| | biphenyl]-3-yl)methoxy)-2-(((((S)-5-oxopyrrolidin-2-yl)methyl)amino)methyl)phenoxy)methyl)nicotinonitrile | |
| 25 | 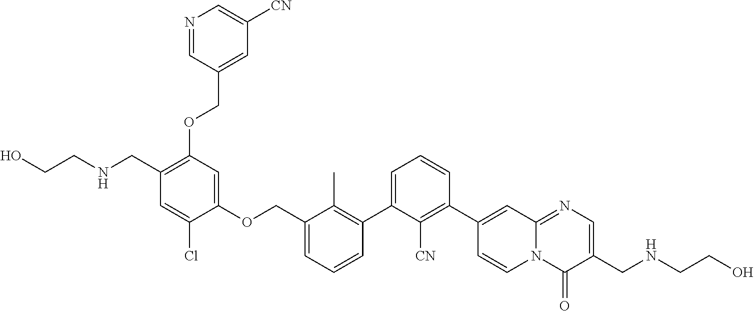 [SMILES: Cc1c(COc2cc(OCc3cncc(C#N)c3)c(CNCCO)cc2Cl)cccc1-c1cccc(-c2ccn3c(=O)c(CNCCO)cnc3c2)c1C#N] | 0.0003 |
| | 5-((4-chloro-5-((2′-cyano-3′-(3-(((2-hydroxyethyl)amino)methyl)-4-oxo-4H-pyrido[1,2-a]pyrimidin-8-yl)-methyl-[1,1′- | |
| | biphenyl]-3-yl)methoxy)-2-(((2-hydroxyethyl)amino)methyl)phenoxy)methyl)nicotinonitrile | |
| 26 | 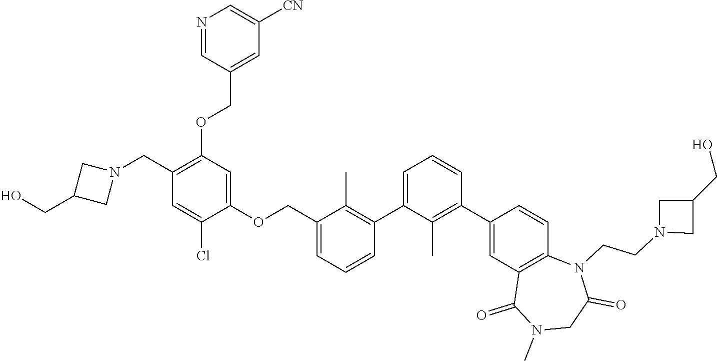 [SMILES: Cc1c(COc2cc(OCc3cncc(C#N)c3)c(CN3CC(CO)C3)cc2Cl)cccc1-c1cccc(-c2ccc3c(c2)C(=O)N(C)CC(=O)N3CCN2CC(CO)C2)c1C] | 0.0007 |
| | 5-((4-chloro-5-((3′-(1-(2-(3-(hydroxymethyl)azetidin-1-yl)ethyl)-4-methyl-2,5-dioxo-2,3,4,5-tetrahydro-1H- | |
| | benzo[e][1,4]diazepin-7-yl)-2,2′-dimethyl-[1,1′-biphenyl]-3-yl)methoxy)-2-((3-(hydroxymethyl)azetidin-1- | |
| | yl)methyl)phenoxy)methyl)nicotinonitrile | |
| 27 | 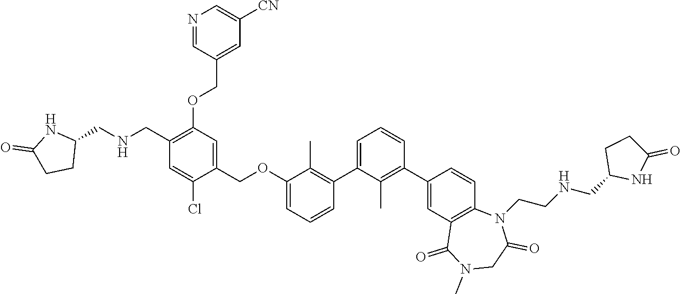 [SMILES: Cc1c(OCc2cc(OCc3cncc(C#N)c3)c(CNC[C@@H]3CCC(=O)N3)cc2Cl)cccc1-c1cccc(-c2ccc3c(c2)C(=O)N(C)CC(=O)N3CCNC[C@@H]2CCC(=O)N2)c1C] | 0.0099 |
| | 5-((4-chloro-5-(((2,2′-dimethyl-3′-(4-methyl-2,5-dioxo-1-(2-((((S)-5-oxopyrrolidin-2-yl)methyl)amino)ethyl)- | |
| | 2,3,4,5-tetrahydro-1H-benzo[e][1,4]diazepin-7-yl)-[1,1′-biphenyl]-3-yl)oxy)methyl)-2-(((((S)-5-oxopyrrolidin-2- | |
| | yl)methyl)amino)methyl)phenoxy)methyl)nicotinonitrile | |
| 28 | 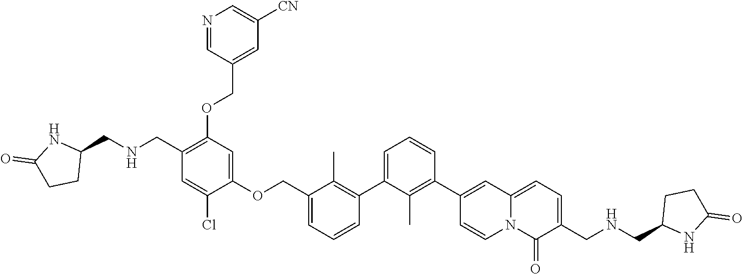 [SMILES: Cc1c(COc2cc(OCc3cncc(C#N)c3)c(CNC[C@H]3CCC(=O)N3)cc2Cl)cccc1-c1cccc(-c2ccn3c(=O)c(CNC[C@H]4CCC(=O)N4)ccc3c2)c1C] | 0.0007 |
| | 5-((4-chloro-5-((2,2′-dimethyl-3′-(4-oxo-3-(((((R)-5-oxopyrrolidin-2-yl)methyl)amino)methyl)-4H-quinolizin-8-yl)-[1,1′- | |
| | biphenyl]-3-yl)methoxy)-2-(((((R)-5-oxopyrrolidin-2-yl)methyl)amino)methyl)phenoxy)methyl)nicotinonitrile | |
| 29 | 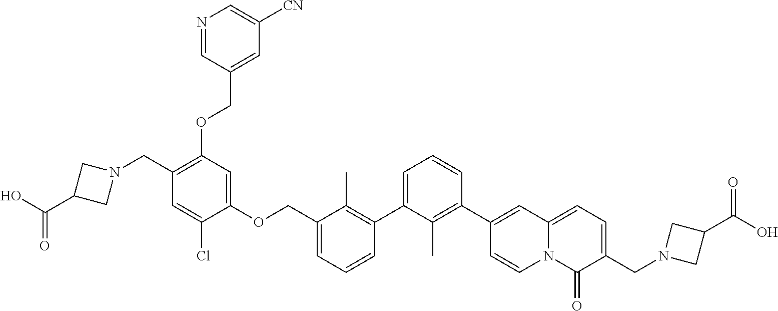 [SMILES: Cc1c(COc2cc(OCc3cncc(C#N)c3)c(CN3CC(C(=O)O)C3)cc2Cl)cccc1-c1cccc(-c2ccn3c(=O)c(CN4CC(C(=O)O)C4)ccc3c2)c1C] | 0.0009 |
| | 1-((8-(3′-((4-((3-carboxyazetidin-1-yl)methyl)-2-chloro-5-((5-cyanopyridin-3-yl)methoxy)phenoxy)methyl)-2,2′-dimethyl-[1,1′- | |
| | biphenyl]-3-yl)-4-oxo-4H-quinolizin-3-yl)methyl)azetidine-3-carboxylic acid | |
| 30 | 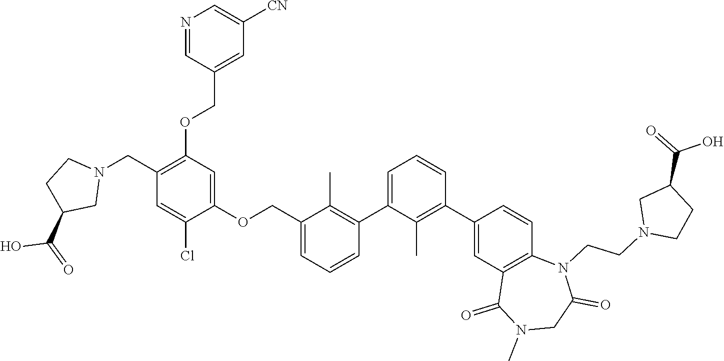 [SMILES: Cc1c(COc2cc(OCc3cncc(C#N)c3)c(CN3CC[C@H](C(=O)O)C3)cc2Cl)cccc1-c1cccc(-c2ccc3c(c2)C(=O)N(C)CC(=O)N3CCN2CC[C@H](C(=O)O)C2)c1C] | 0.0003 |
| | (S)-1-(4-((3′-(1-(2-((S)-3-carboxypyrrolidin-1-yl)ethyl)-4-methyl-2,5-dioxo-2,3,4,5-tetrahydro-1H-benzo[e][1,4]diazepin-7- | |
| | yl)-2,2′-dimethyl-[1,1′-biphenyl]-3-yl)methoxy)-5-chloro-2-((5-cyanopyridin-3-yl)methoxy)benzyl)pyrrolidine-3- | |
| | carboxylic acid | |
| 31 | 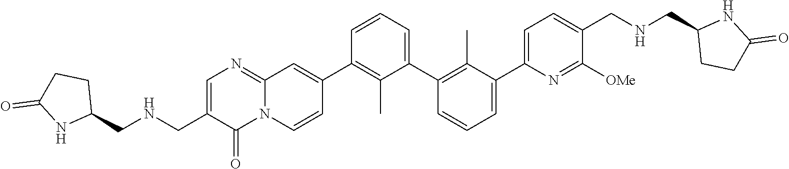 [SMILES: COc1nc(-c2cccc(-c3cccc(-c4ccn5c(=O)c(CNC[C@@H]6CCC(=O)N6)cnc5c4)c3C)c2C)ccc1CNC[C@@H]1CCC(=O)N1] | 0.0023 |
| | 8-(3′-(6-methoxy-5-(((((S)-5-oxopyrrolidin-2-yl)methyl)amino)methyl)pyridin-2-yl)-2,2′-dimethyl-[1,1′-biphenyl]-3-yl)-3- | |
| | (((((S)-5-oxopyrrolidin-2-yl)methyl)amino)methyl)-4H-pyrido[1,2-a]pyrimidin-4-one | |
| 32 | 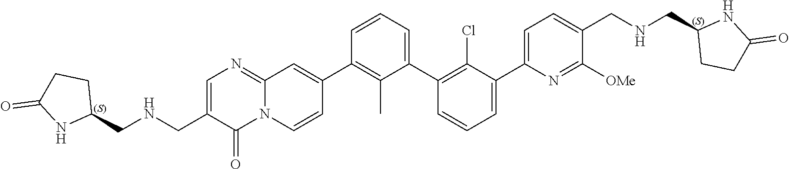 | 0.0023 |
| | 8-(2′-chloro-3′-(6-methoxy-5-(((((S)-5-oxopyrrolidin-2-yl)methyl)amino)methyl)pyridin-2-yl)-2-methyl-[1,1′-biphenyl]-3-yl)-3- | |
| | (((((S)-5-oxopyrrolidin-2-yl)methyl)amino)methyl)-4H-pyrido[1,2-a]pyrimidin-4-one | |
| 33 | 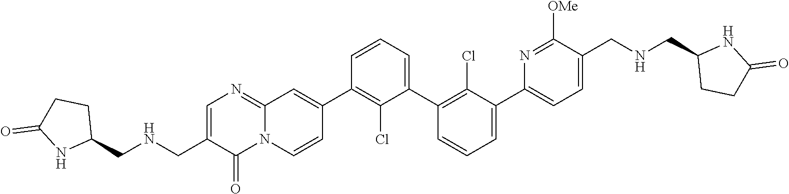 [SMILES: COc1nc(-c2cccc(-c3cccc(-c4ccn5c(=O)c(CNC[C@@H]6CCC(=O)N6)cnc5c4)c3Cl)c2Cl)ccc1CNC[C@@H]1CCC(=O)N1] | 0.0027 |
| | 8-(2,2′-dichloro-3′-(6-methoxy-5-(((((S)-5-oxopyrrolidin-2-yl)methyl)amino)methyl)pyridin-2-yl)-[1,1′-biphenyl]-3-yl)-3-(((((S)-5- | |
| | oxopyrrolidin-2-yl)methyl)amino)methyl)-4H-pyrido[1,2-a]pyrimidin-4-one | |
| 34 | 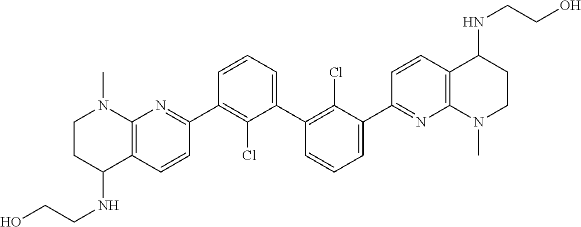 [SMILES: CN1CCC(NCCO)c2ccc(-c3cccc(-c4cccc(-c5ccc6c(n5)N(C)CCC6NCCO)c4Cl)c3Cl)nc21] | 0.0048 |
| | 2,2′-(((2,2′-dichloro-[1,1′-biphenyl]-3,3′-diyl)bis(1-methyl-1,2,3,4-tetrahydro-1,8-naphthyridine- | |
| | 7,4-diyl))bis(azanediyl))bis(ethan-1-ol) mixture of 2 diastereoisomers | |
| 35 | 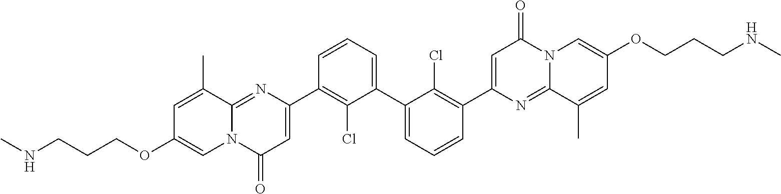 | 0.35 |
| | 2,2′-(2,2′-dichloro-[1,1′-biphenyl]-3,3′-diyl)bis(9-methyl-7-(3-(methylamino)propoxy)-4H-pyrido[1,2-a]pyrimidin-4-one) | |
| 36 | 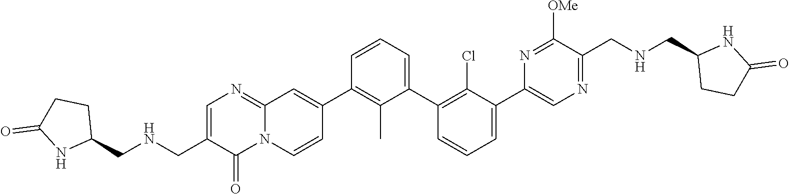 [SMILES: COc1nc(-c2cccc(-c3cccc(-c4ccn5c(=O)c(CNC[C@@H]6CCC(=O)N6)cnc5c4)c3C)c2Cl)cnc1CNC[C@@H]1CCC(=O)N1] | 0.0015 |
| | 8-(2′-chloro-3′-(6-methoxy-5-(((((S)-5-oxopyrrolidin-2-yl)methyl)amino)methyl)pyrazin-2-yl)-2-methyl-[1,1′-biphenyl]-3-yl)-3-(((((S)- | |
| | 5-oxopyrrolidin-2-yl)methyl)amino)methyl)-4H-pyrido[1,2-a]pyrimidin-4-one | |
| 37 | 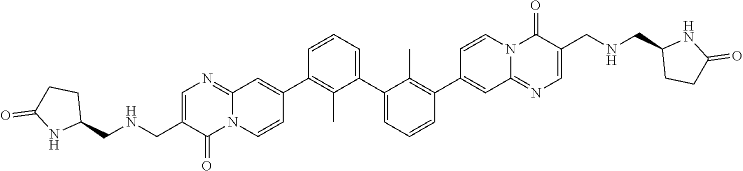 [SMILES: Cc1c(-c2ccn3c(=O)c(CNC[C@@H]4CCC(=O)N4)cnc3c2)cccc1-c1cccc(-c2ccn3c(=O)c(CNC[C@@H]4CCC(=O)N4)cnc3c2)c1C] | 0.14 |
| | 8,8′-(2,2′-dimethyl-[1,1′-biphenyl]-3,3′-diyl)bis(3-(((((S)-5-oxopyrrolidin-2-yl)methyl)amino)methyl)-4H-pyrido[1,2-a]pyrimidin- | |
| | 4-one) | |
| 38 | 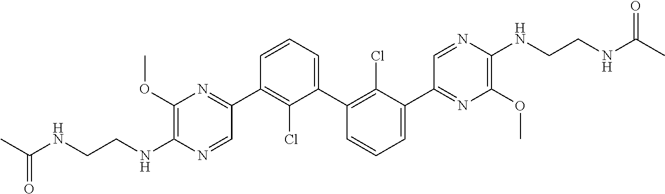 [SMILES: COc1nc(-c2cccc(-c3cccc(-c4cnc(NCCNC(C)=O)c(OC)n4)c3Cl)c2Cl)cnc1NCCNC(C)=O] | 0.02 |
| | N,N′-(((2,2′-dichloro-[1,1′-biphenyl]-3,3′-diyl)bis(3-methoxypyrazine-5,2-diyl))bis(azanediyl))bis(ethane-2,1- | |
| | diyl))diacetamide | |
| 39 | 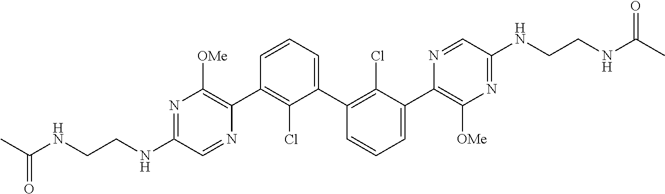 [SMILES: COc1nc(NCCNC(C)=O)cnc1-c1cccc(-c2cccc(-c3ncc(NCCNC(C)=O)nc3OC)c2Cl)c1Cl] | 1.00 |
| | N,N′-((((2,2′-dichloro-[1,1′-biphenyl]-3,3′-diyl)bis(6-methoxypyrazine-5,2-diyl))bis(azanediyl))bis(ethane- | |
| | 2,1-diyl))diacetamide | |
| 40 | 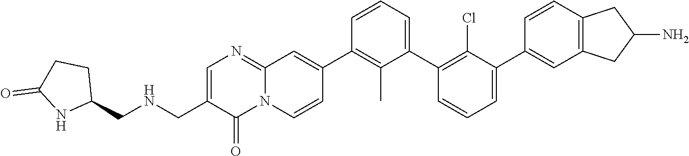 [SMILES: Cc1c(-c2ccn3c(=O)c(CNC[C@@H]4CCC(=O)N4)cnc3c2)cccc1-c1cccc(-c2ccc3c(c2)CC(N)C3)c1Cl] | 0.061 |
| | 8-(3′-(2-amino-2,3-dihydro-1H-inden-5-yl)-2′-chloro-2-methyl-[1,1′-biphenyl]-3-yl)-3-(((((S)-5-oxopyrrolidin-2- | |
| | yl)methyl)amino)methyl)-4H-pyrido[1,2-a]pyrimidin-4-one | |
| | mixture of 2 diastereoisomers | |
| 41 | 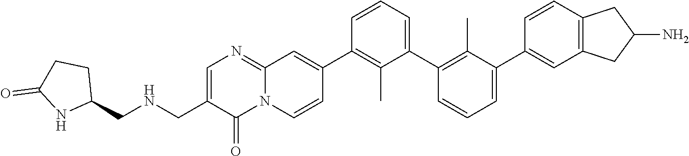 [SMILES: Cc1c(-c2ccc3c(c2)CC(N)C3)cccc1-c1cccc(-c2ccn3c(=O)c(CNC[C@@H]4CCC(=O)N4)cnc3c2)c1C] | 0.093 |
| | 8-(3′-(2-amino-2,3-dihydro-1H-inden-5-yl)-2,2′-dimethyl-[1,1′-biphenyl]-3-yl)-3-(((((S)-5-oxopyrrolidin-2- | |
| | yl)methyl)amino)methyl)-4H-pyrido[1,2-a]pyrimidin-4-one | |
| | mixture of 2 diastereoisomers | |
| 42 | 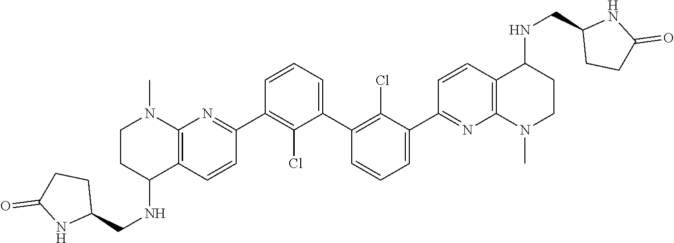 [SMILES: CN1CCC(NC[C@@H]2CCC(=O)N2)c2ccc(-c3cccc(-c4cccc(-c5ccc6c(n5)N(C)CCC6NC[C@@H]5CCC(=O)N5)c4Cl)c3Cl)nc21] | 0.0023 |
| | (5S,5′S)-5,5′-((((2,2′-dichloro-[1,1′-biphenyl]-3,3′-diyl)bis(1-methyl-1,2,3,4-tetrahydro-1,8-naphthyridine-7,4- | |
| | diyl))bis(azanediyl))bis(methylene))bis(pyrrolidin-2-one) | |
| | mixture of 2 diastereoisomers | |
| 43 | 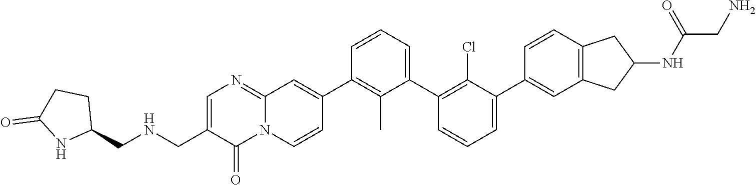 [SMILES: Cc1c(-c2ccn3c(=O)c(CNC[C@@H]4CCC(=O)N4)cnc3c2)cccc1-c1cccc(-c2ccc3c(c2)CC(NC(=O)CN)C3)c1Cl] | 0.27 |
| | 2-amino-N-(5-(2-chloro-2′-methyl-3′-(4-oxo-3-(((((S)-5-oxopyrrolidin-2-yl)methyl)amino)methyl)-4H-pyrido[1,2-a]pyrimidin-8- | |
| | yl)-[1,1′-biphenyl]-3-yl)-2,3-dihydro-1H-inden-2-yl)acetamide | |
| | mixture of 2 diastereoisomers | |
| 44 | 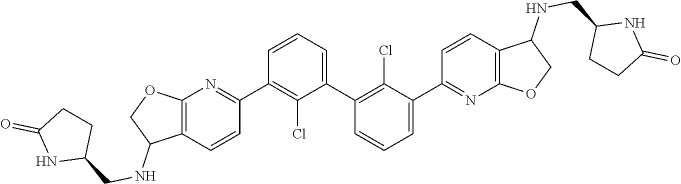 [SMILES: O=C1CC[C@@H](CNC2COc3nc(-c4cccc(-c5cccc(-c6ccc7c(n6)OCC7NC[C@@H]6CCC(=O)N6)c5Cl)c4Cl)ccc32)N1] | 0.55 |
| | (5S,5′S)-5,5′-((((2,2′-dichloro-[1,1′-biphenyl]-3,3′-diyl)bis(2,3-dihydrofuro[2,3-b]pyridine-6,3- | |
| | diyl))bis(azanediyl))bis(methylene))bis(pyrrolidin-2-one) | |
| | mixture of 2 diastereoisomers | |
| 45 | 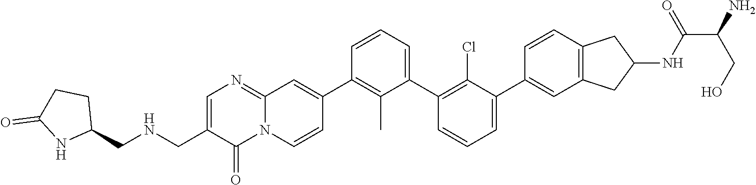 [SMILES: Cc1c(-c2ccn3c(=O)c(CNC[C@@H]4CCC(=O)N4)cnc3c2)cccc1-c1cccc(-c2ccc3c(c2)CC(NC(=O)[C@@H](N)CO)C3)c1Cl] | 0.68 |
| | (2S)-2-amino-N-(5-(2-chloro-2′-methyl-3′-(4-oxo-3-(((((S)-5-oxopyrrolidin-2-yl)methyl)amino)methyl)-4H- | |
| | pyrido[1,2-a]pyrimidin-8-yl)-[1,1′-biphenyl]-3-yl)-2,3-dihydro-1H-inden-2-yl)-3-hydroxypropanamide | |
| | mixture of 2 diastereoisomers | |
| 46 | 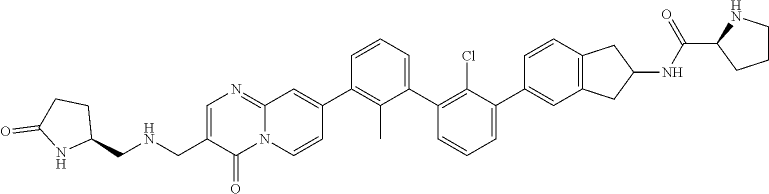 [SMILES: Cc1c(-c2ccn3c(=O)c(CNC[C@@H]4CCC(=O)N4)cnc3c2)cccc1-c1cccc(-c2ccc3c(c2)CC(NC(=O)[C@@H]2CCCN2)C3)c1Cl] | 0.52 |
| | (2S)-N-(5-(2-chloro-2′-methyl-3′-(4-oxo-3-(((((S)-5-oxopyrrolidin-2-yl)methyl)amino)methyl)-4H-pyrido[1,2-a]pyrimidin-8-yl)- | |
| | [1,1′-biphenyl]-3-yl)-2,3-dihydro-1H-inden-2-yl)pyrrolidine-2-carboxamide | |
| | mixture of 2 diastereoisomers | |
| 47 | 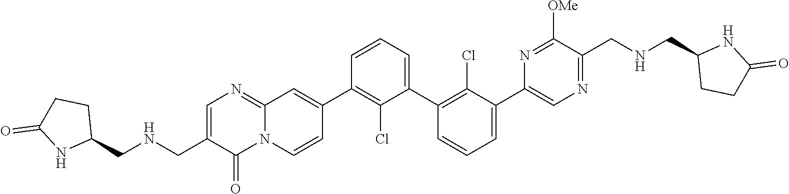 [SMILES: COc1nc(-c2cccc(-c3cccc(-c4ccn5c(=O)c(CNC[C@@H]6CCC(=O)N6)cnc5c4)c3Cl)c2Cl)cnc1CNC[C@@H]1CCC(=O)N1] | 0.0014 |
| | 8-(2,2′-dichloro-3′-(6-methoxy-5-(((((S)-5-oxopyrrolidin-2-yl)methyl)amino)methyl)pyrazin-2-yl)-[1,1′-biphenyl]-3-yl)-3-(((((S)-5- | |
| | oxopyrrolidin-2-yl)methyl)amino)methyl)-4H-pyrido[1,2-a]pyrimidin-4-one | |
| 48 | 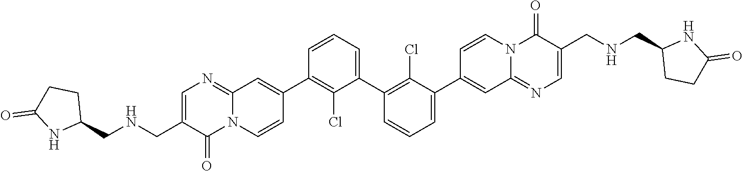 [SMILES: O=C1CC[C@@H](CNCc2cnc3cc(-c4cccc(-c5cccc(-c6ccn7c(=O)c(CNC[C@@H]8CCC(=O)N8)cnc7c6)c5Cl)c4Cl)ccn3c2=O)N1] | 0.007 |
| | 8,8′-(2,2′-dichloro-[1,1′-biphenyl]-3,3′-diyl)bis(3-(((((S)-5-oxopyrrolidin-2-yl)methyl)amino)methyl)-4H-pyridor[1,2-a]pyrimidin- | |
| | 4-one) | |
| 49 | 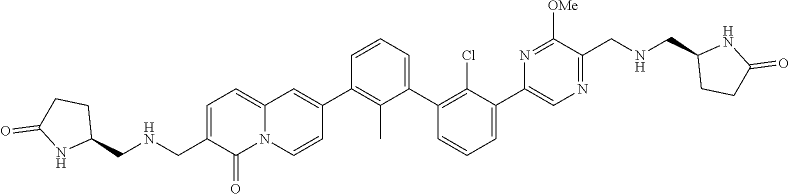 [SMILES: COc1nc(-c2cccc(-c3cccc(-c4ccn5c(=O)c(CNC[C@@H]6CCC(=O)N6)ccc5c4)c3C)c2Cl)cnc1CNC[C@@H]1CCC(=O)N1] | 0.0063 |
| | 8-(2′-chloro-3′-(6-methoxy-5-(((((S)-5-oxopyrrolidin-2-yl)methyl)amino)methyl)pyrazin-2-yl)-2-methyl-[1,1′-biphenyl]-3-yl)-3- | |
| | (((((S)-5-oxopyrrolidin-2-yl)methyl)amino)methyl)-4H-quinolizin-4-one | |
| 50 | 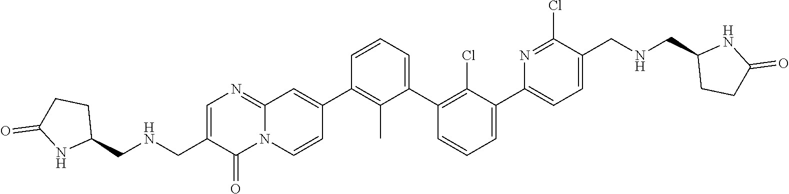 [SMILES: Cc1c(-c2ccn3c(=O)c(CNC[C@@H]4CCC(=O)N4)cnc3c2)cccc1-c1cccc(-c2ccc(CNC[C@@H]3CCC(=O)N3)c(Cl)n2)c1Cl] | 0.0081 |
| | 8-(2′-chloro-3′-(6-chloro-5-(((((S)-5-oxopyrrolidin-2-yl)methyl)amino)methyl)pyridin-2-yl)-2-methyl-[1,1′-biphenyl]-3-yl)-3-(((((S)- | |
| | 5-oxopyrrolidin-2-yl)methyl)amino)methyl)-4H-pyrido[1,2-a]pyrimidin-4-one | |
| 51 | 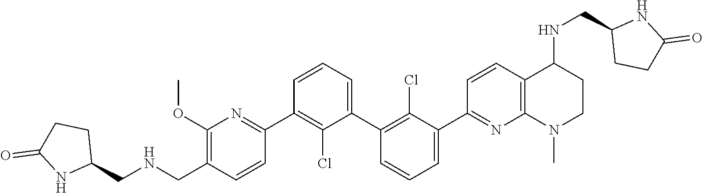 [SMILES: COc1nc(-c2cccc(-c3cccc(-c4ccc5c(n4)N(C)CCC5NC[C@@H]4CCC(=O)N4)c3Cl)c2Cl)ccc1CNC[C@@H]1CCC(=O)N1] | 0.0029 |
| | (5S)-5-(((7-(2,2′-dichloro-3′-(6-methoxy-5-(((((S)-5-oxopyrrolidin-2-yl)methyl)amino)methyl)pyridin-2-yl)-[1,1′- | |
| | biphenyl]-3-yl)-1-methyl-1,2,3,4-tetrahydro-1,8-naphthyridin-4-yl)amino)methyl)pyrrolidin-2-one | |
| | mixture of 2 diastereoisomers | |
| 52 | 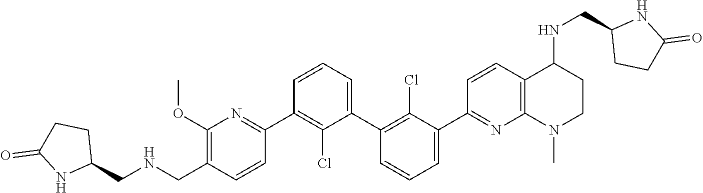 [SMILES: COc1nc(-c2cccc(-c3cccc(-c4ccc5c(n4)N(C)CCC5NC[C@@H]4CCC(=O)N4)c3Cl)c2Cl)ccc1CNC[C@@H]1CCC(=O)N1] | 0.0008 |
| | (5S)-5-(((7-(2,2′-dichloro-3′-(6-methoxy-5-(((((S)-5-oxopyrrolidin-2-yl)methyl)amino)methyl)pyridin-2-yl)-[1,1′- | |
| | biphenyl]-3-yl)-1-methyl-1,2,3,4-tetrahydro-1,8-naphthyridin-4-yl)amino)methyl)pyrrolidin-2-one | |
| | diastereoisomer I | |
| 53 | 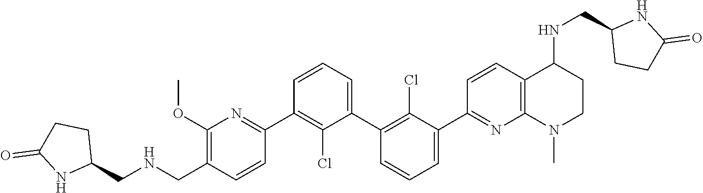 [SMILES: COc1nc(-c2cccc(-c3cccc(-c4ccc5c(n4)N(C)CCC5NC[C@@H]4CCC(=O)N4)c3Cl)c2Cl)ccc1CNC[C@@H]1CCC(=O)N1] | 0.0017 |
| | (5S)-5-(((7-(2,2′-dichloro-3′-(6-methoxy-5-(((((S)-5-oxopyrrolidin-2-yl)methyl)amino)methyl)pyridin-2-yl)-[1,1′- | |
| | biphenyl]-3-yl)-1-methyl-1,2,3,4-tetrahydro-1,8-naphthyridin-4-yl)amino)methyl)pyrrolidin-2-one | |
| | diastereoisomer II | |
| 54 | 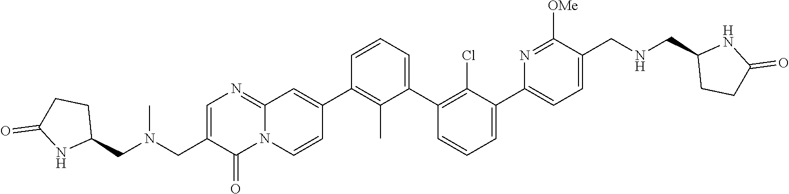 [SMILES: COc1nc(-c2cccc(-c3cccc(-c4ccn5c(=O)c(CN(C)C[C@@H]6CCC(=O)N6)cnc5c4)c3C)c2Cl)ccc1CNC[C@@H]1CCC(=O)N1] | 0.007 |
| | 8-(2′-chloro-3′-(6-methoxy-5-(((((S)-5-oxopyrrolidin-2-yl)methyl)amino)methyl)pyridin-2-yl)-2-methyl-[1,1′-biphenyl]-3-yl)-3- | |
| | ((methyl(((S)-5-oxopyrrolidin-2-yl)methyl)amino)methyl)-4H-pyrido[1,2-a]pyrimidin-4-one | |
| 55 | 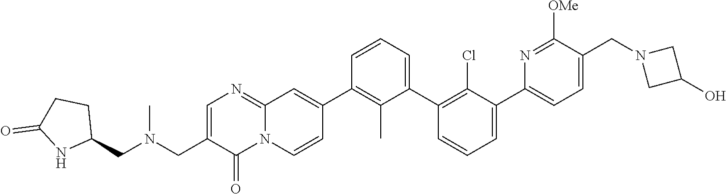 [SMILES: COc1nc(-c2cccc(-c3cccc(-c4ccn5c(=O)c(CN(C)C[C@@H]6CCC(=O)N6)cnc5c4)c3C)c2Cl)ccc1CN1CC(O)C1] | 0.043 |
| | (S)-8-(2′-chloro-3′-(5-((3-hydroxyazetidin-1-yl)methyl)-6-methoxypyridin-2-yl)-2-methyl-[1,1′-biphenyl]-3-yl)-3- | |
| | ((methyl((5-oxopyrrolidin-2-yl)methyl)amino)methyl)-4H-pyrido[1,2-a]pyrimidin-4-one | |
| 56 | 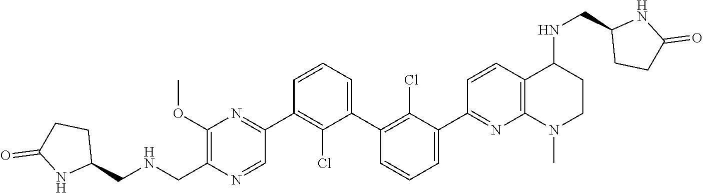 [SMILES: COc1nc(-c2cccc(-c3cccc(-c4ccc5c(n4)N(C)CCC5NC[C@@H]4CCC(=O)N4)c3Cl)c2Cl)cnc1CNC[C@@H]1CCC(=O)N1] | 0.0007 |
| | (5S)-5-(((7-(2,2′-dichloro-3′-(6-methoxy-5-(((((S)-5-oxopyrrolidin-2-yl)methyl)amino)methyl)pyrazin-2-yl)-[1,1′- | |
| | biphenyl]-3-yl)-1-methyl-1,2,3,4-tetrahydro-1,8-naphthyridin-4-yl)amino)methyl)pyrrolidin-2-one | |
| | mixture of 2 diastereoisomers | |
| 57 | 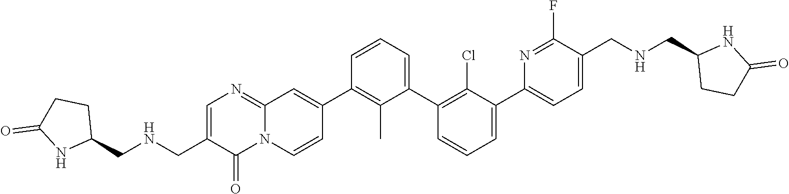 [SMILES: Cc1c(-c2ccn3c(=O)c(CNC[C@@H]4CCC(=O)N4)cnc3c2)cccc1-c1cccc(-c2ccc(CNC[C@@H]3CCC(=O)N3)c(F)n2)c1Cl] | 0.0031 |
| | 8-(2′-chloro-3′-(6-fluoro-5-(((((S)-5-oxopyrrolidin-2-yl)methyl)amino)methyl)pyridin-2-yl)-2-methyl-[1,1′-biphenyl]-3-yl)-3- | |
| | (((((S)-5-oxopyrrolidin-2-yl)methyl)amino)methyl)-4H-pyrido[1,2-a]pyrimidin-4-one | |
| 58 | 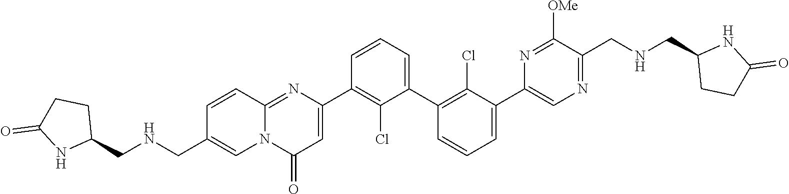 [SMILES: COc1nc(-c2cccc(-c3cccc(-c4cc(=O)n5cc(CNC[C@@H]6CCC(=O)N6)ccc5n4)c3Cl)c2Cl)cnc1CNC[C@@H]1CCC(=O)N1] | 0.0022 |
| | 2-(2,2′-dichloro-3′-(6-methoxy-5-(((((S)-5-oxopyrrolidin-2-yl)methyl)amino)methyl)pyrazin-2-yl)-[1,1′-biphenyl]-3-yl)-7-(((((S)-5- | |
| | oxopyrrolidin-2-yl)methyl)amino)methyl)-4H-pyrido[1,2-a]pyrimidin-4-one | |
| 59 | 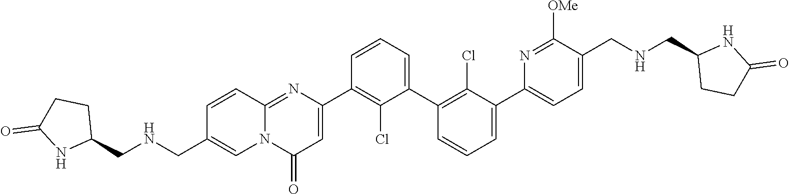 [SMILES: COc1nc(-c2cccc(-c3cccc(-c4cc(=O)n5cc(CNC[C@@H]6CCC(=O)N6)ccc5n4)c3Cl)c2Cl)ccc1CNC[C@@H]1CCC(=O)N1] | 0.0029 |
| | 2-(2,2′-dichloro-3′-(6-methoxy-5-(((((S)-5-oxopyrrolidin-2-yl)methyl)amino)methyl)pyridin-2-yl)-[1,1′-biphenyl]-3-yl)-7-(((((S)-5- | |
| | oxopyrrolidin-2-yl)methyl)amino)methyl)-4H-pyrido[1,2-a]pyrimidin-4-one | |
| 60 | 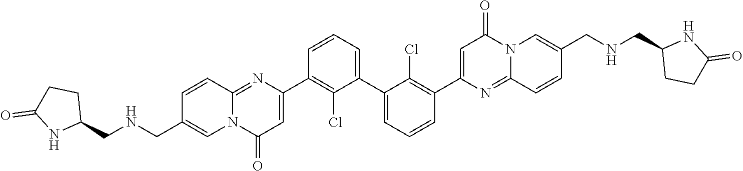 [SMILES: O=C1CC[C@@H](CNCc2ccc3nc(-c4cccc(-c5cccc(-c6cc(=O)n7cc(CNC[C@@H]8CCC(=O)N8)ccc7n6)c5Cl)c4Cl)cc(=O)n3c2)N1] | 0.46 |
| | 2,2′-(2,2′-dichloro-[1,1′-biphenyl]-3,3′-diyl)bis(7-(((((S)-5-oxopyrrolidin-2-yl)methyl)amino)methyl)-4H-pyrido[1,2-a]pyrimidin- | |
| | 4-one) | |
| 61 | 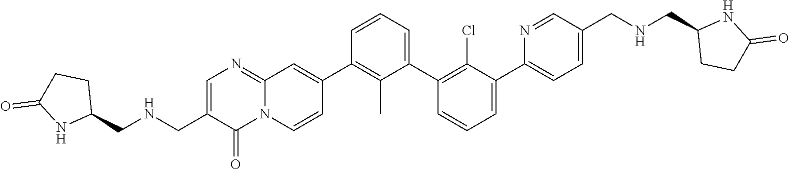 [SMILES: Cc1c(-c2ccn3c(=O)c(CNC[C@@H]4CCC(=O)N4)cnc3c2)cccc1-c1cccc(-c2ccc(CNC[C@@H]3CCC(=O)N3)cn2)c1Cl] | 0.016 |
| | 8-(2′-chloro-2-methyl-3′-(5-(((((S)-5-oxopyrrolidin-2-yl)methyl)amino)methyl)pyridin-2-yl)-[1,1′-biphenyl]-3-yl)-3-(((((S)-5- | |
| | oxopyrrolidin-2-yl)methyl)amino)methyl)-4H-pyrido[1,2-a]pyrimidin-4-one | |
| 62 | 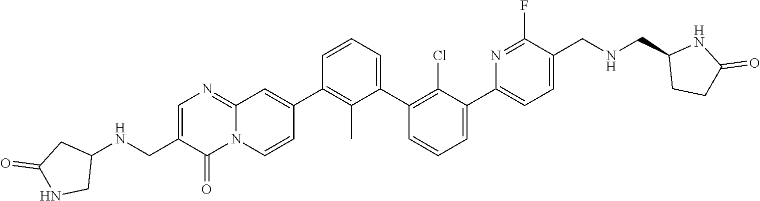 [SMILES: Cc1c(-c2ccn3c(=O)c(CNC4CNC(=O)C4)cnc3c2)cccc1-c1cccc(-c2ccc(CNC[C@@H]3CCC(=O)N3)c(F)n2)c1Cl] | 0.019 |
| | 8-(2′-chloro-3′-(6-fluoro-5-(((((S)-5-oxopyrrolidin-2-yl)methyl)amino)methyl)pyridin-2-yl)-2-methyl-[1,1′-biphenyl]-3-yl)-3-(((5- | |
| | oxopyrrolidin-3-yl)amino)methyl)-4H-pyrido[1,2-a]pyrimidin-4-one | |
| | mixture of 2 diastereoisomers | |
| 63 | 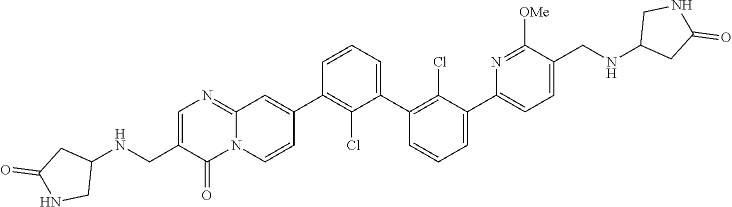 [SMILES: COc1nc(-c2cccc(-c3cccc(-c4ccn5c(=O)c(CNC6CNC(=O)C6)cnc5c4)c3Cl)c2Cl)ccc1CNC1CNC(=O)C1] | 0.0015 |
| | 8-(2,2′-dichloro-3′-(6-methoxy-5-(((5-oxopyrrolidin-3-yl)amino)methyl)pyridin-2-yl)-[1,1′-biphenyl]-3-yl)-3-(((5- | |
| | oxopyrrolidin-3-yl)amino)methyl)-4H-pyrido[1,2-a]pyrimidin-4-one | |
| | mixture of 2 diastereoisomers | |
| 64 | 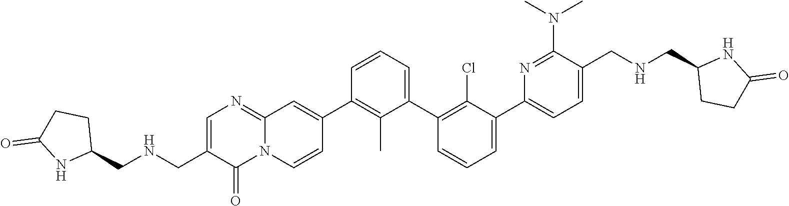 [SMILES: Cc1c(-c2ccn3c(=O)c(CNC[C@@H]4CCC(=O)N4)cnc3c2)cccc1-c1cccc(-c2ccc(CNC[C@@H]3CCC(=O)N3)c(N(C)C)n2)c1Cl] | 0.037 |
| | 8-(2′-chloro-3′-(6-(dimethylamino)-5-(((((S)-5-oxopyrrolidin-2-yl)methyl)amino)methyl)pyridin-2-yl)-2-methyl-[1,1′-biphenyl]-3-yl)- | |
| | 3-(((((S)-5-oxopyrrolidin-2-yl)methyl)amino)methyl)-4H-pyrido[1,2-a]pyrimidin-4-one | |
| 65 | 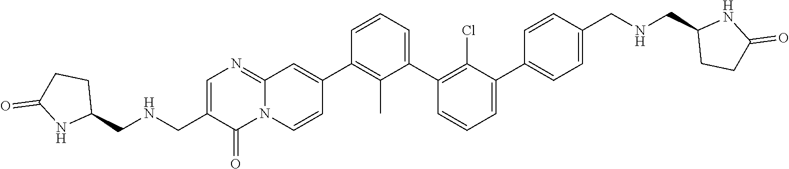 [SMILES: Cc1c(-c2ccn3c(=O)c(CNC[C@@H]4CCC(=O)N4)cnc3c2)cccc1-c1cccc(-c2ccc(CNC[C@@H]3CCC(=O)N3)cc2)c1Cl] | 0.0056 |
| | 8-(2′-chloro-2-methyl-4″-(((((S)-5-oxopyrrolidin-2-yl)methyl)amino)methyl)-[1,1′:3′,1″-terphenyl]-3-yl)-3-(((((S)-5-oxopyrrolidin-2- | |
| | yl)methyl)amino)methyl)-4H-pyrido[1,2-a]pyrimidin-4-one | |
| 66 | 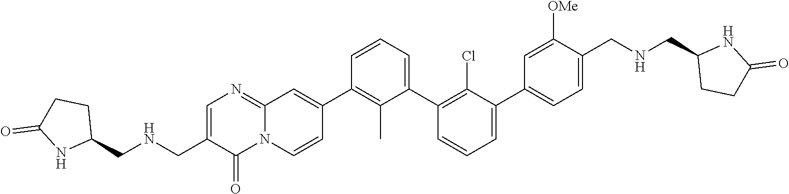 [SMILES: COc1cc(-c2cccc(-c3cccc(-c4ccn5c(=O)c(CNC[C@@H]6CCC(=O)N6)cnc5c4)c3C)c2Cl)ccc1CNC[C@@H]1CCC(=O)N1] | 0.0052 |
| | 8-(2′-chloro-3″-methoxy-2-methyl-4″-(((((S)-5-oxopyrrolidin-2-yl)methyl)amino)methyl)-[1,1′:3′,1″-terphenyl]-3-yl)-3-(((((S)-5- | |
| | oxopyrrolidin-2-yl)methyl)amino)methyl)-4H-pyrido[1,2-a]pyrimidin-4-one | |
| 67 | 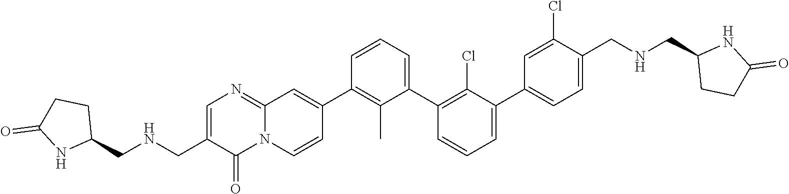 [SMILES: Cc1c(-c2ccn3c(=O)c(CNC[C@@H]4CCC(=O)N4)cnc3c2)cccc1-c1cccc(-c2ccc(CNC[C@@H]3CCC(=O)N3)c(Cl)c2)c1Cl] | 0.0035 |
| | 8-(2′,3″-dichloro-2-methyl-4″-(((((S)-5-oxopyrrolidin-2-yl)methyl)amino)methyl)-[1,1′:3′,1″-terphenyl]-3-yl)-3-(((((S)-5- | |
| | oxopyrrolidin-2-yl)methyl)amino)methyl)-4H-pyrido[1,2-a]pyrimidin-4-one | |
| 68 | 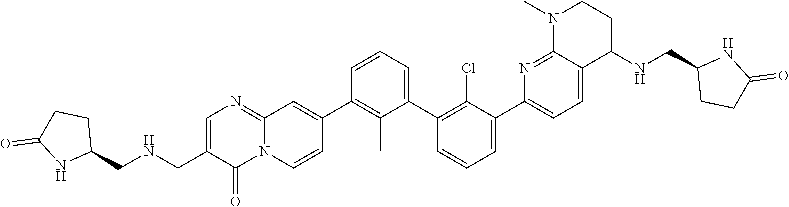 [SMILES: Cc1c(-c2ccn3c(=O)c(CNC[C@@H]4CCC(=O)N4)cnc3c2)cccc1-c1cccc(-c2ccc3c(n2)N(C)CCC3NC[C@@H]2CCC(=O)N2)c1Cl] | 0.011 |
| | 8-(2′-chloro-2-methyl-3′-(8-methyl-5-((((S)-5-oxopyrrolidin-2-yl)methyl)amino)-5,6,7,8-tetrahydro-1,8-naphthyridin-2-yl)-[1,1′- | |
| | biphenyl]-3-yl)-3-(((((S)-5-oxopyrrolidin-2-yl)methyl)amino)methyl)-4H-pyrido[1,2-a]pyrimidin-4-one | |
| 69 | 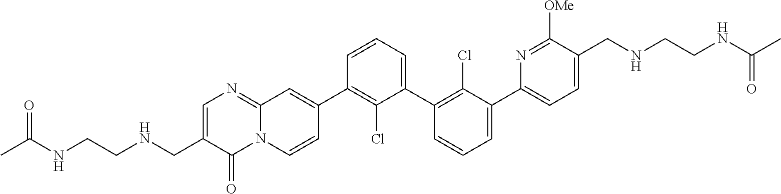 [SMILES: COc1nc(-c2cccc(-c3cccc(-c4ccn5c(=O)c(CNCCNC(C)=O)cnc5c4)c3Cl)c2Cl)ccc1CNCCNC(C)=O] | 0.0019 |
| | N-(2-(((6-(3′-(3-(((2-acetamidoethyl)amino)methyl)-4-oxo-4H-pyrido[1,2-a]pyrimidin-8-yl)-2,2′-dichloro-[1,1′-biphenyl]-3-yl)-2- | |
| | methoxypyridin-3-yl)methyl)amino)ethyl)acetamide | |
| 70 | 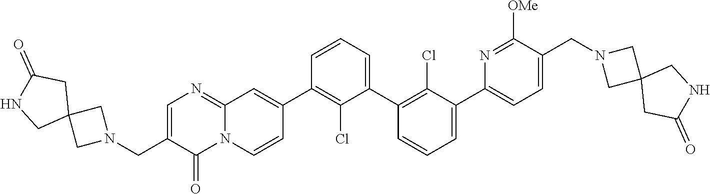 [SMILES: COc1nc(-c2cccc(-c3cccc(-c4ccn5c(=O)c(CN6CC7(CNC(=O)C7)C6)cnc5c4)c3Cl)c2Cl)ccc1CN1CC2(CNC(=O)C2)C1] | 0.0071 |
| | 8-(2,2′-dichloro-3′-(6-methoxy-5-((7-oxo-2,6-diazaspiro[3.4]octan-2-yl)methyl)pyridin-2-yl)-[1,1′-biphenyl]-3-yl)-3- | |
| | ((7-oxo-2,6-diazaspiro[3.4]octan-2-yl)methyl)-4H-pyrido[1,2-a]pyrimidin-4-one | |
| 71 | 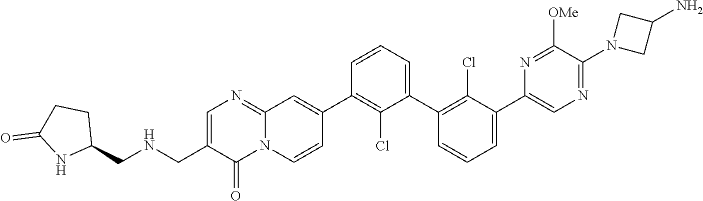 [SMILES: COc1nc(-c2cccc(-c3cccc(-c4ccn5c(=O)c(CNC[C@@H]6CCC(=O)N6)cnc5c4)c3Cl)c2Cl)cnc1N1CC(N)C1] | 0.002 |
| | (S)-8-(3′-(5-(3-aminoazetidin-1-yl)-6-methoxypyrazin-2-yl)-2,2′-dichloro-[1,1′-biphenyl]-3-yl)-3-((((5-oxopyrrolidin-2- | |
| | yl)methyl)amino)methyl)-4H-pyrido[1,2-a]pyrimidin-4-one | |
| 72 | 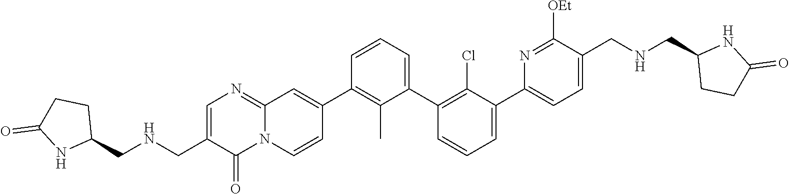 [SMILES: CCOc1nc(-c2cccc(-c3cccc(-c4ccn5c(=O)c(CNC[C@@H]6CCC(=O)N6)cnc5c4)c3C)c2Cl)ccc1CNC[C@@H]1CCC(=O)N1] | 0.0094 |
| | 8-(2′-chloro-3′-(6-ethoxy-5-(((((S)-5-oxopyrrolidin-2-yl)methyl)amino)methyl)pyridin-2-yl)-2-methyl-[1,1′-biphenyl]-3-yl)-3- | |
| | (((((S)-5-oxopyrrolidin-2-yl)methyl)amino)methyl)-4H-pyrido[1,2-a]pyrimidin-4-one | |
| 73 | 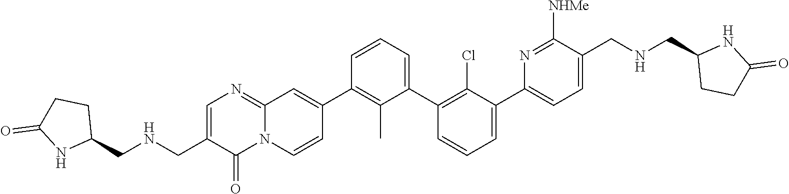 [SMILES: CNc1nc(-c2cccc(-c3cccc(-c4ccn5c(=O)c(CNC[C@@H]6CCC(=O)N6)cnc5c4)c3C)c2Cl)ccc1CNC[C@@H]1CCC(=O)N1] | 0.0063 |
| | 8-(2′-chloro-2-methyl-3′-(6-(methylamino)-5-(((((S)-5-oxopyrrolidin-2-yl)methyl)amino)methyl)pyridin-2-yl)-[1,1′-biphenyl]-3-yl)-3- | |
| | (((((S)-5-oxopyrrolidin-2-yl)methyl)amino)methyl)-4H-pyrido[1,2-a]pyrimidin-4-one | |
| 74 | 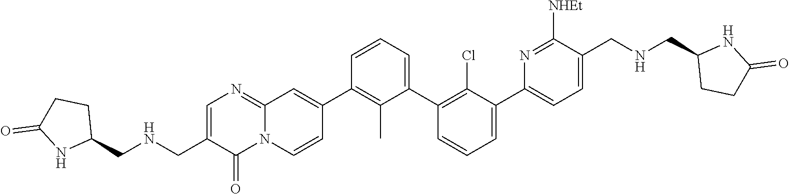 | 0.015 |
| | 8-(2′-chloro-3′-(6-(ethylamino)-5-(((((S)-5-oxopyrrolidin-2-yl)methyl)amino)methyl)pyridin-2-yl)-2-methyl-[1,1′-biphenyl]-3-yl)-3- | |
| | (((((S)-5-oxopyrrolidin-2-yl)methyl)amino)methyl)-4H-pyrido[1,2-a]pyrimidin-4-one | |
| 75 | 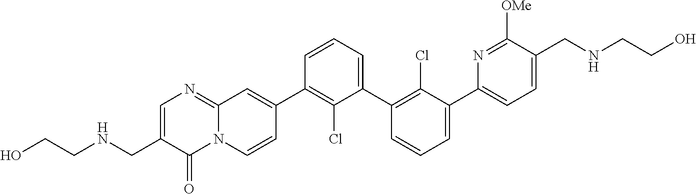 [SMILES: COc1nc(-c2cccc(-c3cccc(-c4ccn5c(=O)c(CNCCO)cnc5c4)c3Cl)c2Cl)ccc1CNCCO] | 0.0034 |
| | 8-(2,2′-dichloro-3′-(5-(((2-hydroxyethyl)amino)methyl)-6-methoxypyridin-2-yl)-[1,1′-biphenyl]-3-yl)-3-(((2- | |
| | hydroxyethyl)amino)methyl)-4H-pyrido[1,2-a]pyrimidin-4-one | |
| 76 | 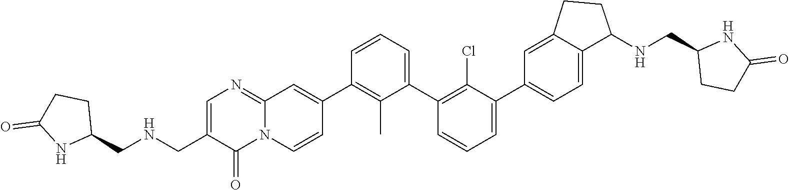 [SMILES: Cc1c(-c2ccn3c(=O)c(CNC[C@@H]4CCC(=O)N4)cnc3c2)cccc1-c1cccc(-c2ccc3c(c2)CCC3NC[C@@H]2CCC(=O)N2)c1Cl] | 0.0034 |
| | 8-(2′-chloro-2-methyl-3′-(1-((((S)-5-oxopyrrolidin-2-yl)methyl)amino)-2,3-dihydro-1H-inden-5-yl)-[1,1′-biphenyl]-3-yl)-3-(((((S)-5- | |
| | oxopyrrolidin-2-yl)methyl)amino)methyl)-4H-pyrido[1,2-a]pyrimidin-4-one | |
| | mixture of 2 diastereoisomers | |
| 77 | 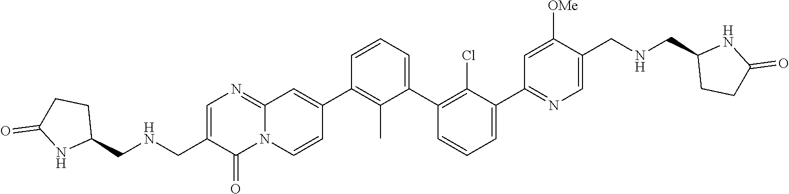 [SMILES: COc1cc(-c2cccc(-c3cccc(-c4ccn5c(=O)c(CNC[C@@H]6CCC(=O)N6)cnc5c4)c3C)c2Cl)ncc1CNC[C@@H]1CCC(=O)N1] | 0.0063 |
| | 8-(2′-chloro-3′-(4-methoxy-5-(((((S)-5-oxopyrrolidin-2-yl)methyl)amino)methyl)pyridin-2-yl)-2-methyl-[1,1′-biphenyl]-3-yl)-3- | |
| | (((((S)-5-oxopyrrolidin-2-yl)methyl)amino)methyl)-4H-pyrido[1,2-a]pyrimidin-4-one | |
| 78 | 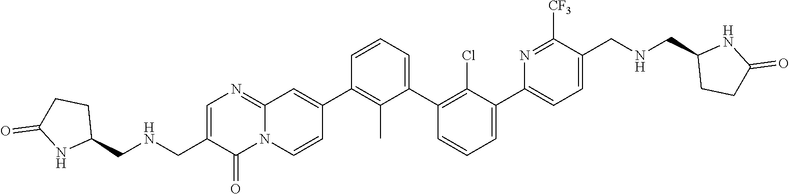 [SMILES: Cc1c(-c2ccn3c(=O)c(CNC[C@@H]4CCC(=O)N4)cnc3c2)cccc1-c1cccc(-c2ccc(CNC[C@@H]3CCC(=O)N3)c(C(F)(F)F)n2)c1Cl] | 0.016 |
| | 8-(2′-chloro-2-methyl-3′-(5-(((((S)-5-oxopyrrolidin-2-yl)methyl)amino)methyl)-6-(trifluoromethyl)pyridin-2-yl)-[1,1′-biphenyl]-3- | |
| | yl)-3-(((((S)-5-oxopyrrolidin-2-yl)methyl)amino)methyl)-4H-pyrido[1,2-a]pyrimidin-4-one | |
| 79 | 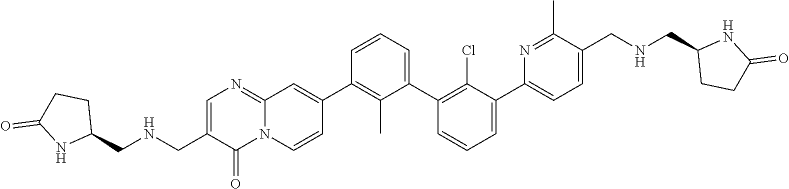 [SMILES: Cc1nc(-c2cccc(-c3cccc(-c4ccn5c(=O)c(CNC[C@@H]6CCC(=O)N6)cnc5c4)c3C)c2Cl)ccc1CNC[C@@H]1CCC(=O)N1] | 0.0028 |
| | 8-(2′-chloro-2-methyl-3′-(6-methyl-5-(((((S)-5-oxopyrrolidin-2-yl)methyl)amino)methyl)pyridin-2-yl)-[1,1′-biphenyl]-3-yl)-3-(((((S)- | |
| | 5-oxopyrrolidin-2-yl)methyl)amino)methyl)-4H-pyrido[l,2-a]pyrimidin-4-one | |
| 80 | 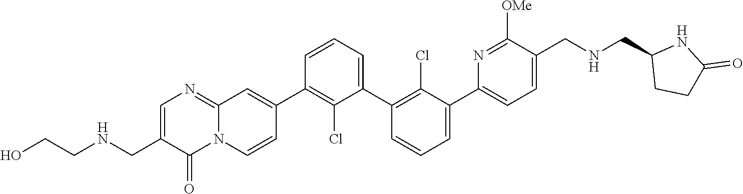 [SMILES: COc1nc(-c2cccc(-c3cccc(-c4ccn5c(=O)c(CNCCO)cnc5c4)c3Cl)c2Cl)ccc1CNC[C@@H]1CCC(=O)N1] | 0.0042 |
| | (S)-8-(2,2′-dichloro-3′-(6-methoxy-5-((((5-oxopyrrolidin-2-yl)methyl)amino)methyl)pyridin-2-yl)-[1,1′-biphenyl]-3-yl)-3-(((2- | |
| | hydroxyethyl)amino)methyl)-4H-pyrido[1,2-a]pyrimidin-4-one | |
| 81 | 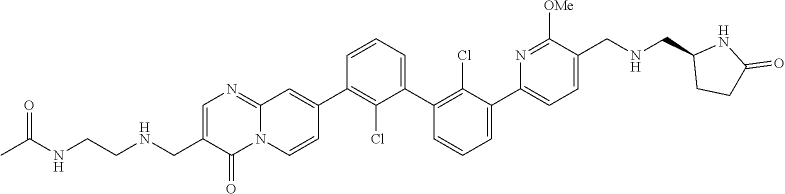 [SMILES: COc1nc(-c2cccc(-c3cccc(-c4ccn5c(=O)c(CNCCNC(C)=O)cnc5c4)c3Cl)c2Cl)ccc1CNC[C@@H]1CCC(=O)N1] | 0.0083 |
| | (S)-N-(2-(((8-(2,2′-dichloro-3′-(6-methoxy-5-((((5-oxopyrrolidin-2-yl)methyl)amino)methyl)pyridin-2-yl)-[1,1′-biphenyl]-3-yl)-4- | |
| | oxo-4H-pyrido[1,2-a]pyrimidin-3-yl)methyl)amino)ethyl)acetamide | |
| 82 | 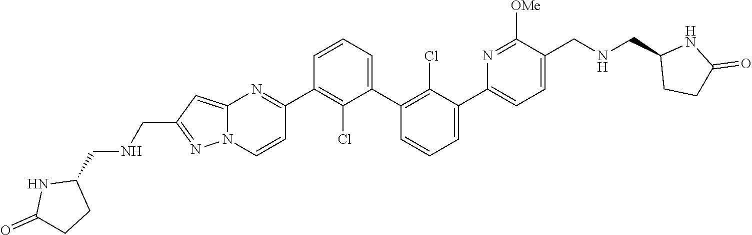 [SMILES: COc1nc(-c2cccc(-c3cccc(-c4ccn5nc(CNC[C@@H]6CCC(=O)N6)cc5n4)c3Cl)c2Cl)ccc1CNC[C@@H]1CCC(=O)N1] | 0.0099 |
| | (S)-5-((((6-(2,2′-dichloro-3′-(2-(((((S)-5-oxopyrrolidin-2-yl)methyl)amino)methyl)pyrazolo[1,5-a]pyrimidin-5-yl)-[1,1′- | |
| | biphenyl]-3-yl)-2-methoxypyridin-3-yl)methyl)amino)methyl)pyrrolidin-2-one | |
| 83 | 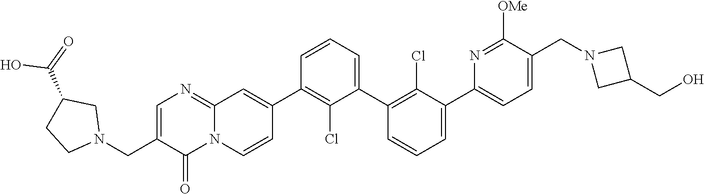 [SMILES: COc1nc(-c2cccc(-c3cccc(-c4ccn5c(=O)c(CN6CC[C@H](C(=O)O)C6)cnc5c4)c3Cl)c2Cl)ccc1CN1CC(CO)C1] | 0.0004 |
| | (S)-1-((8-(2,2′-dichloro-3′-(5-((3-(hydroxymethyl)azetidin-1-yl)methyl)-6-methoxypyridin-2-yl)-[1,1′-biphenyl]-3-yl)-4- | |
| | oxo-4H-pyrido[1,2-a]pyrimidin-3-yl)methyl)pyrrolidine-3-carboxylic acid | |
| 84 | 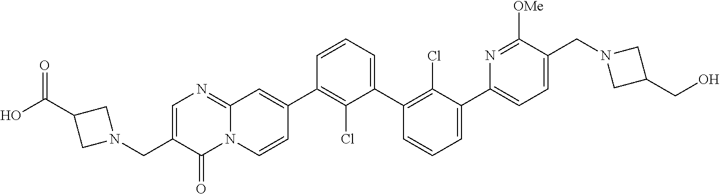 [SMILES: COc1nc(-c2cccc(-c3cccc(-c4ccn5c(=O)c(CN6CC(C(=O)O)C6)cnc5c4)c3Cl)c2Cl)ccc1CN1CC(CO)C1] | 0.0003 |
| | 1-((8-(2,2′-dichloro-3′-(5-((3-(hydroxymethyl)azetidin-1-yl)methyl)-6-methoxypyridin-2-yl)-[1,1′-biphenyl]-3-yl)-4-oxo- | |
| | 4H-pyrido[1,2-a]pyrimidin-3-yl)methyl)azetidine-3-carboxylic acid | |
| 85 | 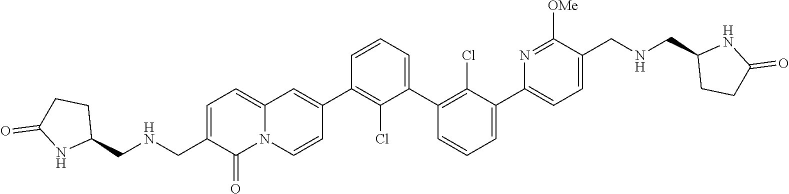 [SMILES: COc1nc(-c2cccc(-c3cccc(-c4ccn5c(=O)c(CNC[C@@H]6CCC(=O)N6)ccc5c4)c3Cl)c2Cl)ccc1CNC[C@@H]1CCC(=O)N1] | 0.0025 |
| | 8-(2,2′-dichloro-3′-(6-methoxy-5-(((((S)-5-oxopyrrolidin-2-yl)methyl)amino)methyl)pyridin-2-yl)-[1,1′-biphenyl]-3-yl)-3-(((((S)-5- | |
| | oxopyrrolidin-2-yl)methyl)amino)methyl)-4H-quinolizin-4-one | |
| 86 | 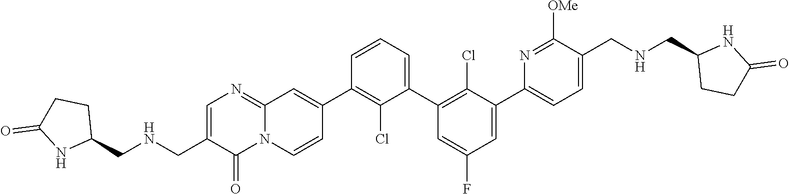 [SMILES: COc1nc(-c2cc(F)cc(-c3cccc(-c4ccn5c(=O)c(CNC[C@@H]6CCC(=O)N6)cnc5c4)c3Cl)c2Cl)ccc1CNC[C@@H]1CCC(=O)N1] | 0.0041 |
| | 8-(2,2′-dichloro-5′-fluoro-3′-(6-methoxy-5-(((((S)-5-oxopyrrolidin-2-yl)methyl)amino)methyl)pyridin-2-yl)-[1,1′-biphenyl]-3-yl)-3- | |
| | (((((S)-5-oxopyrrolidin-2-yl)methyl)amino)methyl)-4H-pyrido[1,2-a]pyrimidin-4-one | |
| 87 | 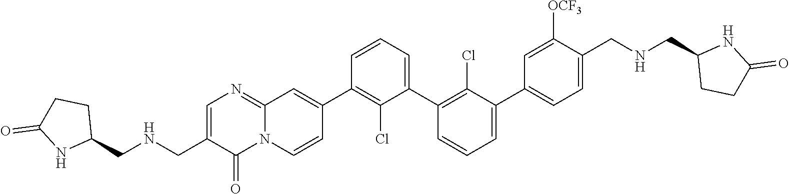 [SMILES: O=C1CC[C@@H](CNCc2ccc(-c3cccc(-c4cccc(-c5ccn6c(=O)c(CNC[C@@H]7CCC(=O)N7)cnc6c5)c4Cl)c3Cl)cc2OC(F)(F)F)N1] | 0.0026 |
| | 8-(2,2′-dichloro-4″-(((((S)-5-oxopyrrolidin-2-yl)methyl)amino)methyl)-3″-(trifluoromethoxy)-[1,1′:3′,1″-terphenyl]-3-yl)-3-(((((S)-5- | |
| | oxopyrrolidin-2-yl)methyl)amino)methyl)-4H-pyrido[1,2-a]pyrimidin-4-one | |
| 88 | 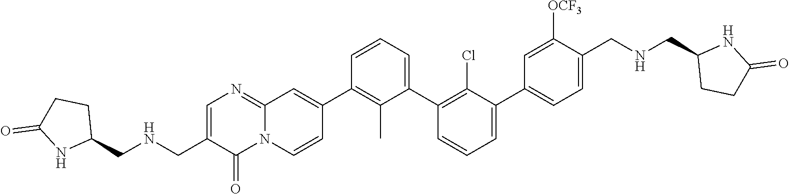 [SMILES: Cc1c(-c2ccn3c(=O)c(CNC[C@@H]4CCC(=O)N4)cnc3c2)cccc1-c1cccc(-c2ccc(CNC[C@@H]3CCC(=O)N3)c(OC(F)(F)F)c2)c1Cl] | 0.011 |
| | 8-(2′-chloro-2-methyl-4″-(((((S)-5-oxopyrrolidin-2-yl)methyl)amino)methyl)-3″-(trifluoromethoxy)-[1,1′:3′,1″-terphenyl]-3-yl)-3- | |
| | (((((S)-5-oxopyrrolidin-2-yl)methyl)amino)methyl)-4H-pyrido[1,2-a]pyrimidin-4-one | |
| 89 | 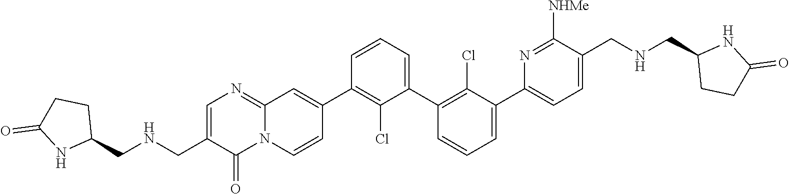 [SMILES: CNc1nc(-c2cccc(-c3cccc(-c4ccn5c(=O)c(CNC[C@@H]6CCC(=O)N6)cnc5c4)c3Cl)c2Cl)ccc1CNC[C@@H]1CCC(=O)N1] | 0.0035 |
| | 8-(2,2′-dichloro-3′-(6-(methylamino)-5-(((((S)-5-oxopyrrolidin-2-yl)methyl)amino)methyl)pyridin-2-yl)-[1,1′-biphenyl]-3-yl)-3-(((((S)- | |
| | 5-oxopyrrolidin-2-yl)methyl)amino)methyl)-4H-pyrido[1,2-a]pyrimidin-4-one | |
| 90 | 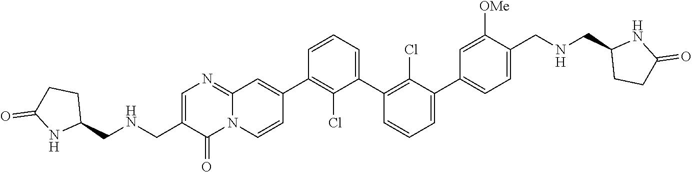 [SMILES: COc1cc(-c2cccc(-c3cccc(-c4ccn5c(=O)c(CNC[C@@H]6CCC(=O)N6)cnc5c4)c3Cl)c2Cl)ccc1CNC[C@@H]1CCC(=O)N1] | 0.0015 |
| | 8-(2,2′-dichloro-3″-methoxy-4″-(((((S)-5-oxopyrrolidin-2-yl)methyl)amino)methyl)-[1,1′:3′,1-terphenyl]-3-yl)-3- | |
| | (((((S)-5-oxopyrrolidin-2-yl)methyl)amino)methyl)-4H-pyrido[1,2-a]pyrimidin-4-one | |
| 91 | 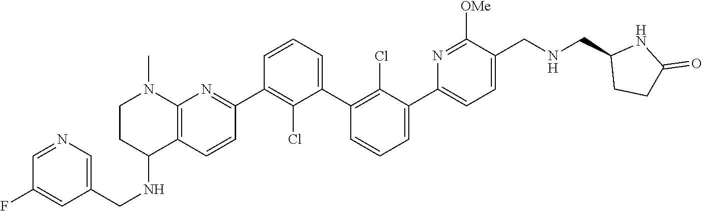 [SMILES: COc1nc(-c2cccc(-c3cccc(-c4ccc5c(n4)N(C)CCC5NCc4cncc(F)c4)c3Cl)c2Cl)ccc1CNC[C@@H]1CCC(=O)N1] | 0.18 |
| | (5S)-5-((((6-(2,2′-dichloro-3′-(5-(((5-fluoropyridin-3-yl)methyl)amino)-8-methyl-5,6,7,8-tetrahydro-1,8-naphthyridin-2- | |
| | yl)-[1,1′-biphenyl]-3-yl)-2-methoxypyridin-3-yl)methyl)amino)methyl)pyrrolidin-2-one | |
| | mixture of 2 diastereoisomers | |
| 92 | 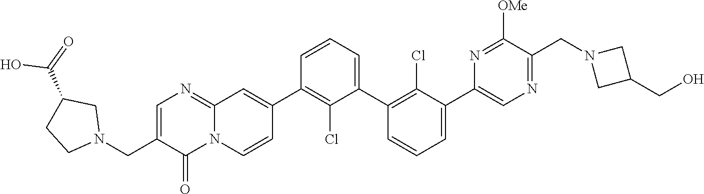 [SMILES: COc1nc(-c2cccc(-c3cccc(-c4ccn5c(=O)c(CN6CC[C@H](C(=O)O)C6)cnc5c4)c3Cl)c2Cl)cnc1CN1CC(CO)C1] | 0.0003 |
| | (S)-1-((8-(2,2′-dichloro-3′-(5-((3-(hydroxymethyl)azetidin-1-yl)methyl)-6-methoxypyrazin-2-yl)-[1,1′-biphenyl]-3-yl)-4- | |
| | oxo-4H-pyrido[1,2-a]pyrimidin-3-yl)methyl)pyrrolidine-3-carboxylic acid | |
| 93 | 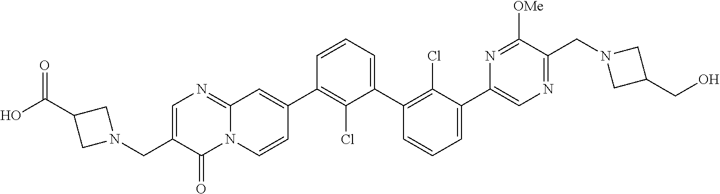 [SMILES: COc1nc(-c2cccc(-c3cccc(-c4ccn5c(=O)c(CN6CC(C(=O)O)C6)cnc5c4)c3Cl)c2Cl)cnc1CN1CC(CO)C1] | 0.0003 |
| | 1-((8-(2,2′-dichloro-3′-(5-((3-(hydroxymethyl)azetidin-1-yl)methyl)-6-methoxypyrazin-2-yl)-[1,1′-biphenyl]-3-yl)-4-oxo- | |
| | 4H-pyrido[1,2-a]pyrimidin-3-yl)methyl)azetidine-3-carboxylic acid | |
| 94 | 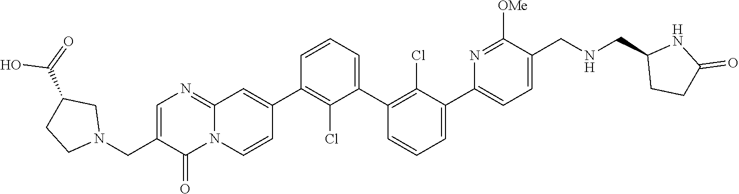 [SMILES: COc1nc(-c2cccc(-c3cccc(-c4ccn5c(=O)c(CN6CC[C@H](C(=O)O)C6)cnc5c4)c3Cl)c2Cl)ccc1CNC[C@@H]1CCC(=O)N1] | 0.0003 |
| | (S)-1-((8-(2,2′-dichloro-3′-(6-methoxy-5-(((((S)-5-oxopyrrolidin-2-yl)methyl)amino)methyl)pyridin-2-yl)-[1,1′-biphenyl]-3- | |
| | yl)-4-oxo-4H-pyrido[1,2-a]pyrimidin-3-yl)methyl)pyrrolidine-3-carboxylic acid | |
| 95 | 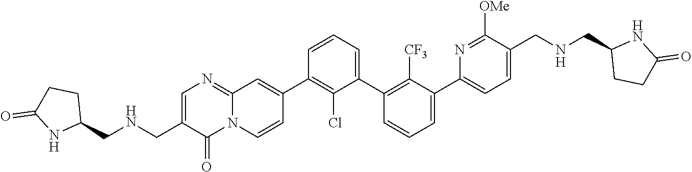 [SMILES: COc1nc(-c2cccc(-c3cccc(-c4ccn5c(=O)c(CNC[C@@H]6CCC(=O)N6)cnc5c4)c3Cl)c2C(F)(F)F)ccc1CNC[C@@H]1CCC(=O)N1] | 0.023 |
| | 8-(2-chloro-3′-(6-methoxy-5-(((((S)-5-oxopyrrolidin-2-yl)methyl)amino)methyl)pyridin-2-yl)-2′-(trifluoromethyl)- | |
| | [1,1′-biphenyl]-3-yl)-3-(((((S)-5-oxopyrrolidin-2-yl)methyl)amino)methyl)-4H-pyrido[1,2-a]pyrimidin-4-one | |
| 96 | 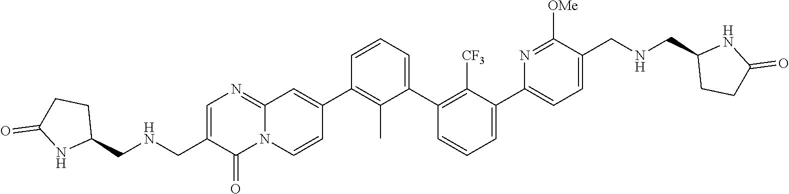 [SMILES: COc1nc(-c2cccc(-c3cccc(-c4ccn5c(=O)c(CNC[C@@H]6CCC(=O)N6)cnc5c4)c3C)c2C(F)(F)F)ccc1CNC[C@@H]1CCC(=O)N1] | 0.068 |
| | 8-(3′-(6-methoxy-5-(((((S)-5-oxopyrrolidin-2-yl)methyl)amino)methyl)pyridin-2-yl)-2-methyl-2′-(trifluoromethyl)-[1,1′-biphenyl]- | |
| | 3-yl)-3-(((((S)-5-oxopyrrolidin-2-yl)methyl)amino)methyl)-4H-pyrido[1,2-a]pyrimidin-4-one | |
| 97 | 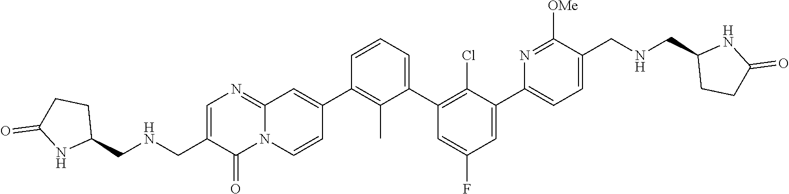 [SMILES: COc1nc(-c2cc(F)cc(-c3cccc(-c4ccn5c(=O)c(CNC[C@@H]6CCC(=O)N6)cnc5c4)c3C)c2Cl)ccc1CNC[C@@H]1CCC(=O)N1] | 0.0051 |
| | 8-(2′-chloro-5′-fluoro-3′-(6-methoxy-5-(((((S)-5-oxopyrrolidin-2-yl)methyl)amino)methyl)pyridin-2-yl)-2-methyl-[1,1′-biphenyl]-3- | |
| | yl)-3-(((((S)-5-oxopyrrolidin-2-yl)methyl)amino)methyl)-4H-pyrido[1,2-a]pyrimidin-4-one | |
| 98 | 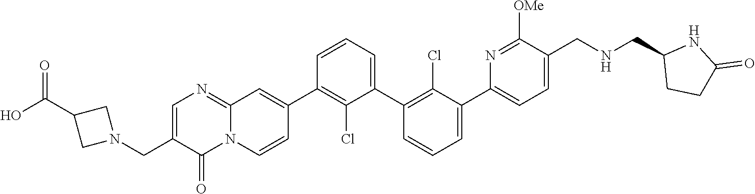 [SMILES: COc1nc(-c2cccc(-c3cccc(-c4ccn5c(=O)c(CN6CC(C(=O)O)C6)cnc5c4)c3Cl)c2Cl)ccc1CNC[C@@H]1CCC(=O)N1] | 0.0003 |
| | (S)-1-((8-(2,2′-dichloro-3′-(6-methoxy-5-((((5-oxopyrrolidin-2-yl)methyl)amino)methyl)pyridin-2-yl)-[1,1′-biphenyl]-3-yl)-4- | |
| | oxo-4H-pyrido[1,2-a]pyrimidin-3-yl)methyl)azetidine-3-carboxylic acid | |
| 99 | 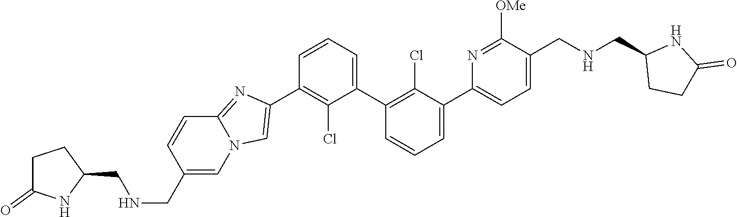 [SMILES: COc1nc(-c2cccc(-c3cccc(-c4cn5cc(CNC[C@@H]6CCC(=O)N6)ccc5n4)c3Cl)c2Cl)ccc1CNC[C@@H]1CCC(=O)N1] | 0.0048 |
| | (S)-5-((((6-(2,2′-dichloro-3′-(6-(((((S)-5-oxopyrrolidin-2-yl)methyl)amino)methyl)imidazo[1,2-a]pyridin-2-yl)-[1,1′- | |
| | biphenyl]-3-yl)-2-methoxypyridin-3-yl)methyl)amino)methyl)pyrrolidin-2-one | |
| 100 | 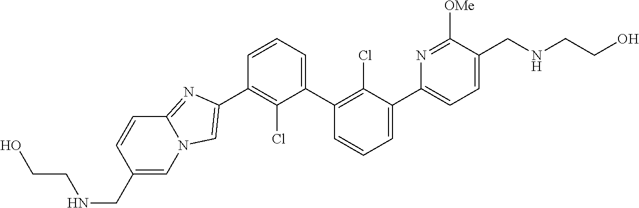 [SMILES: COc1nc(-c2cccc(-c3cccc(-c4cn5cc(CNCCO)ccc5n4)c3Cl)c2Cl)ccc1CNCCO] | 0.0043 |
| | 2-(((2-(2,2′-dichloro-3′-(5-(((2-hydroxyethyl)amino)methyl)-6-methoxypyridin-2-yl)-[1,1′-biphenyl]-3- | |
| | yl)imidazo[1,2-a]pyridin-6-yl)methyl)amino)ethan-1-ol | |
| 101 | 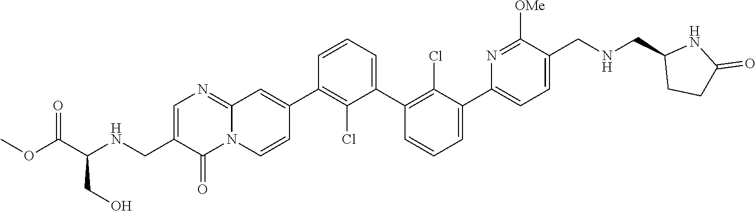 [SMILES: COC(=O)[C@H](CO)NCc1cnc2cc(-c3cccc(-c4cccc(-c5ccc(CNC[C@@H]6CCC(=O)N6)c(OC)n5)c4Cl)c3Cl)ccn2c1=O] | 0.0021 |
| | methyl ((8-(2,2′-dichloro-3′-(6-methoxy-5-(((((S)-5-oxopyrrolidin-2-yl)methyl)amino)methyl)pyridin-2-yl)-[1,1′-biphenyl]-3- | |
| | yl)-4-oxo-4H-pyrido[1,2-a]pyrimidin-3-yl)methyl)-L-serinate | |
| 102 | 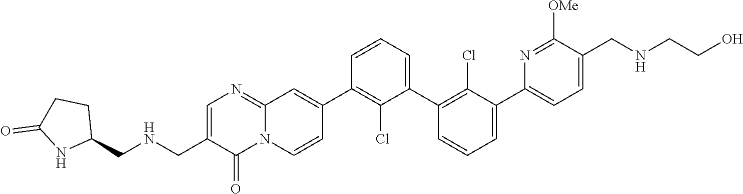 [SMILES: COc1nc(-c2cccc(-c3cccc(-c4ccn5c(=O)c(CNC[C@@H]6CCC(=O)N6)cnc5c4)c3Cl)c2Cl)ccc1CNCCO] | 0.0071 |
| | (S)-8-(2,2′-dichloro-3′-(5-(((2-hydroxyethyl)amino)methyl)-6-methoxypyridin-2-yl)-[1,1′-biphenyl]-3-yl)-3-((((5- | |
| | oxopyrrolidin-2-yl)methyl)amino)methyl)-4H-pyrido[1,2-a]pyrimidin-4-one | |
| 103 | 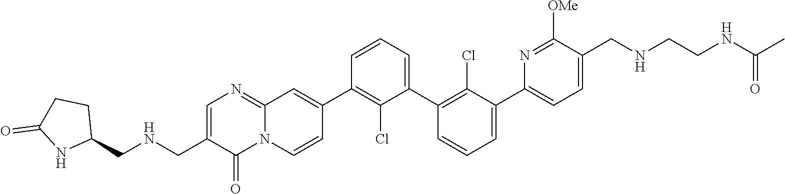 [SMILES: COc1nc(-c2cccc(-c3cccc(-c4ccn5c(=O)c(CNC[C@@H]6CCC(=O)N6)cnc5c4)c3Cl)c2Cl)ccc1CNCCNC(C)=O] | 0.005 |
| | (S)-N-(2-(((6-(2,2′-dichloro-3′-(4-oxo-3-((((5-oxopyrrolidin-2-yl)methyl)amino)methyl)-4H-pyrido[1,2-a]pyrimidin-8-yl)-[1,1′- | |
| | biphenyl]-3-yl)-2-methoxypyridin-3-yl)methyl)amino)ethyl)acetamide | |
| 104 | 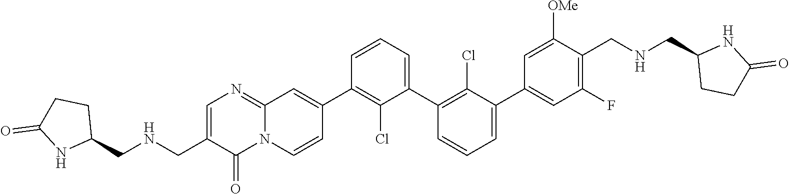 [SMILES: COc1cc(-c2cccc(-c3cccc(-c4ccn5c(=O)c(CNC[C@@H]6CCC(=O)N6)cnc5c4)c3Cl)c2Cl)cc(F)c1CNC[C@@H]1CCC(=O)N1] | 0.0088 |
| | 8-(2,2′-dichloro-3″-fluoro-5″-methoxy-4″-(((((S)-5-oxopyrrolidin-2-yl)methyl)amino)methyl)-[1,1′:3′,1″-terphenyl]-3-yl)-3-(((((S)-5- | |
| | oxopyrrolidin-2-yl)methyl)amino)methyl)-4H-pyrido[1,2-a]pyrimidin-4-one | |
| 105 | 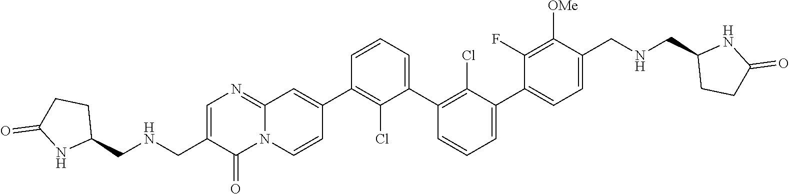 [SMILES: COc1c(CNC[C@@H]2CCC(=O)N2)ccc(-c2cccc(-c3cccc(-c4ccn5c(=O)c(CNC[C@@H]6CCC(=O)N6)cnc5c4)c3Cl)c2Cl)c1F] | 0.0059 |
| | 8-(2,2′-dichloro-2″-fluoro-3″-methoxy-4″-(((((S)-5-oxopyrrolidin-2-yl)methyl)amino)methyl)-[1,1′:3′,1″-terphenyl]-3-yl)-3-(((((S)- | |
| | 5-oxopyrrolidin-2-yl)methyl)amino)methyl)-4H-pyrido[1,2-a]pyrimidin-4-one | |
| 106 | 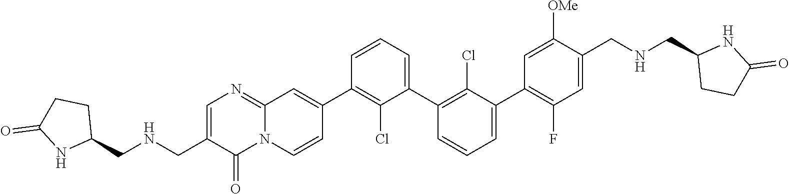 [SMILES: COc1cc(-c2cccc(-c3cccc(-c4ccn5c(=O)c(CNC[C@@H]6CCC(=O)N6)cnc5c4)c3Cl)c2Cl)c(F)cc1CNC[C@@H]1CCC(=O)N1] | 0.0049 |
| | 8-(2,2′-dichloro-2″-fluoro-5″-methoxy-4″-(((((S)-5-oxopyrrolidin-2-yl)methyl)amino)methyl)-[1,1′:3′,1″-terphenyl]-3-yl)-3-(((((S)- | |
| | 5-oxopyrrolidin-2-yl)methyl)amino)methyl)-4H-pyrido[1,2-a]pyrimidin-4-one | |
| 107 | 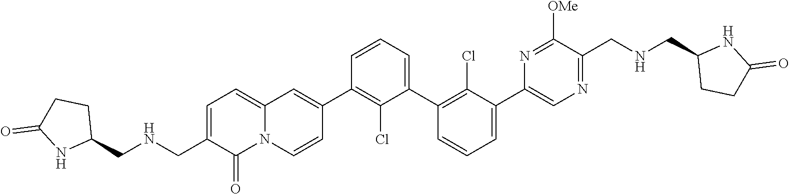 [SMILES: COc1nc(-c2cccc(-c3cccc(-c4ccn5c(=O)c(CNC[C@@H]6CCC(=O)N6)ccc5c4)c3Cl)c2Cl)cnc1CNC[C@@H]1CCC(=O)N1] | 0.0004 |
| | 8-(2,2′-dichloro-3′-(6-methoxy-5-(((((S)-5-oxopyrrolidin-2-yl)methyl)amino)methyl)pyrazin-2-yl)-[1,1′-biphenyl]-3-yl)-3-(((((S)-5- | |
| | oxopyrrolidin-2-yl)methyl)amino)methyl)-4H-quinolizin-4-one | |
| 108 | 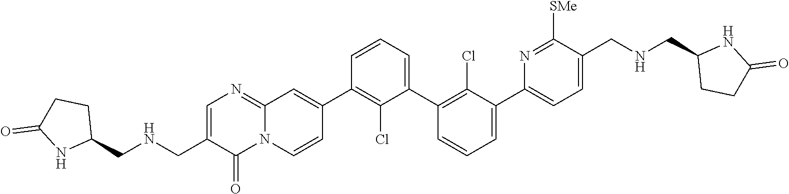 [SMILES: CSc1nc(-c2cccc(-c3cccc(-c4ccn5c(=O)c(CNC[C@@H]6CCC(=O)N6)cnc5c4)c3Cl)c2Cl)ccc1CNC[C@@H]1CCC(=O)N1] | 0.001 |
| | 8-(2,2′-dichloro-3′-(6-(methylthio)-5-(((((S)-5-oxopyrrolidin-2-yl)methyl)amino)methyl)pyridin-2-yl)-[1,1′-biphenyl]-3-yl)-3-(((((S)-5- | |
| | oxopyrrolidin-2-yl)methyl)amino)methyl)-4H-pyrido[1,2-a]pyrimidin-4-one | |
| 109 | 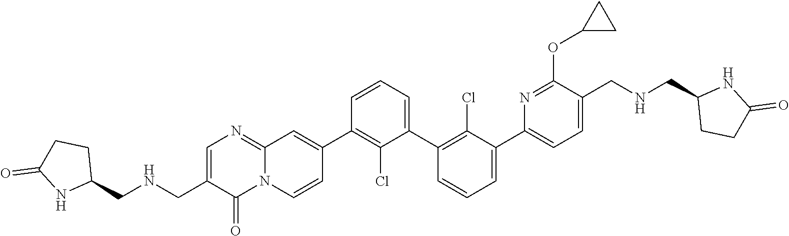 [SMILES: O=C1CC[C@@H](CNCc2ccc(-c3cccc(-c4cccc(-c5ccn6c(=O)c(CNC[C@@H]7CCC(=O)N7)cnc6c5)c4Cl)c3Cl)nc2OC2CC2)N1] | 0.0014 |
| | 8-(2,2′-dichloro-3′-(6-cyclopropoxy-5-(((((S)-5-oxopyrrolidin-2-yl)methyl)amino)methyl)pyridin-2-yl)-[1,1′-biphenyl]-3-yl)-3-(((((S)- | |
| | 5-oxopyrrolidin-2-yl)methyl)amino)methyl)-4H-pyrido[1,2-a]pyrimidin-4-one | |
| 110 | 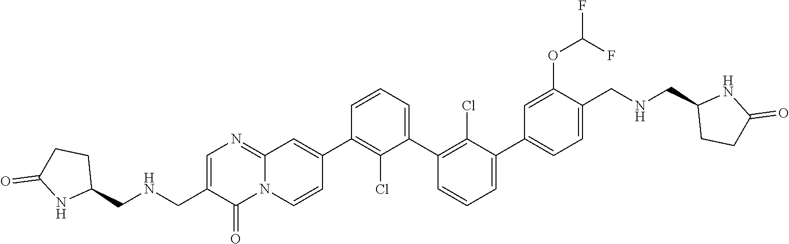 [SMILES: O=C1CC[C@@H](CNCc2ccc(-c3cccc(-c4cccc(-c5ccn6c(=O)c(CNC[C@@H]7CCC(=O)N7)cnc6c5)c4Cl)c3Cl)cc2OC(F)F)N1] | 0.0024 |
| | 8-(2,2′-dichloro-3″-(difluoromethoxy)-4″-(((((S)-5-oxopyrrolidin-2-yl)methyl)amino)methyl)-[1,1′:3′,1″-terphenyl]-3-yl)-3-(((((S)- | |
| | 5-oxopyrrolidin-2-yl)methyl)amino)methyl)-4H-pyrido[1,2-a]pyrimidin-4-one | |
| 111 | 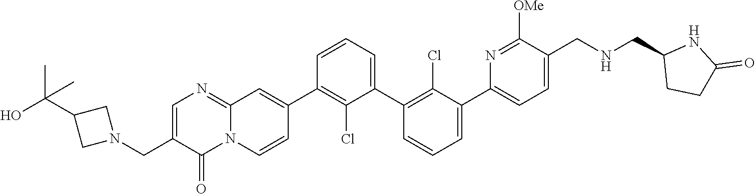 [SMILES: COc1nc(-c2cccc(-c3cccc(-c4ccn5c(=O)c(CN6CC(C(C)(C)O)C6)cnc5c4)c3Cl)c2Cl)ccc1CNC[C@@H]1CCC(=O)N1] | 0.0065 |
| | (S)-8-(2,2′-dichloro-3′-(6-methoxy-5-((((5-oxopyrrolidin-2-yl)methyl)amino)methyl)pyridin-2-yl)-[1,1′-biphenyl]-3-yl)-3-((3-(2- | |
| | hydroxypropan-2-yl)azetidin-1-yl)methyl)-4H-pyrido[1,2-a]pyrimidin-4-one | |
| 112 | 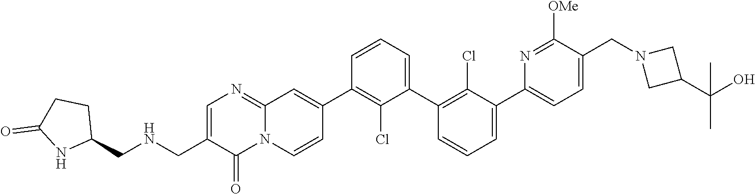 [SMILES: COc1nc(-c2cccc(-c3cccc(-c4ccn5c(=O)c(CNC[C@@H]6CCC(=O)N6)cnc5c4)c3Cl)c2Cl)ccc1CN1CC(C(C)(C)O)C1] | 0.0029 |
| | (S)-8-(2,2′-dichloro-3′-(5-((3-(2-hydroxypropan-2-yl)azetidin-1-yl)methyl)-6-methoxypyridin-2-yl)-[1,1′-biphenyl]-3-yl)-3-((((5- | |
| | oxopyrrolidin-2-yl)methyl)amino)methyl)-4H-pyrido[1,2-a]pyrimidin-4-one | |
| 113 | 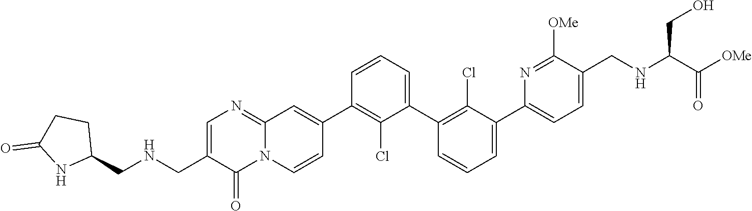 [SMILES: COC(=O)[C@H](CO)NCc1ccc(-c2cccc(-c3cccc(-c4ccn5c(=O)c(CNC[C@@H]6CCC(=O)N6)cnc5c4)c3Cl)c2Cl)nc1OC] | 0.054 |
| | methyl ((6-(2,2′-dichloro-3′-(4-oxo-3-(((((S)-5-oxopyrrolidin-2-yl)methyl)amino)methyl)-4H-pyrido[1,2-a]pyrimidin-8-yl)- | |
| | [1,1′-biphenyl]-3-yl)-2-methoxypyridin-3-yl)methyl)-L-serinate | |
| 114 | 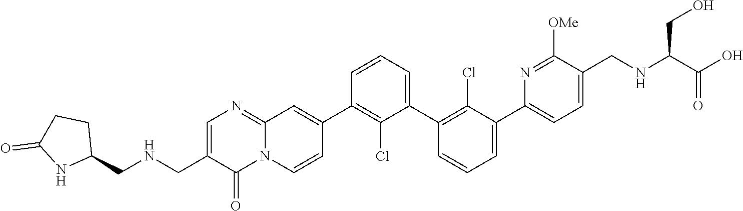 [SMILES: COc1nc(-c2cccc(-c3cccc(-c4ccn5c(=O)c(CNC[C@@H]6CCC(=O)N6)cnc5c4)c3Cl)c2Cl)ccc1CN[C@@H](CO)C(=O)O] | 0.0006 |
| | ((6-(2,2′-dichloro-3′-(4-oxo-3-(((((S)-5-oxopyrrolidin-2-yl)methyl)amino)methyl)-4H-pyrido[1,2-a]pyrimidin-8-yl)-[1,1′- | |
| | biphenyl]-3-yl)-2-methoxypyridin-3-yl)methyl)-L-serine | |
| 115 | 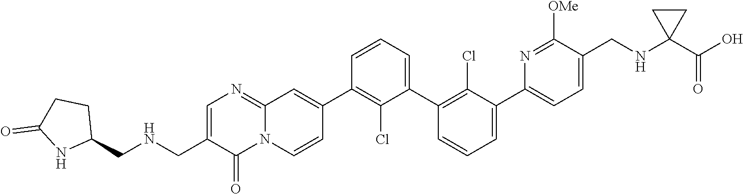 [SMILES: COc1nc(-c2cccc(-c3cccc(-c4ccn5c(=O)c(CNC[C@@H]6CCC(=O)N6)cnc5c4)c3Cl)c2Cl)ccc1CNC1(C(=O)O)CC1] | 0.0025 |
| | (S)-1-(((6-(2,2′-dichloro-3′-(4-oxo-3-((((5-oxopyrrolidin-2-yl)methyl)amino)methyl)-4H-pyrido[1,2-a]pyrimidin-8-yl)-[1,1′- | |
| | biphenyl]-3-yl)-2-methoxypyridin-3-yl)methyl)amino)cyclopropane-1-carboxylic acid | |
| 116 | 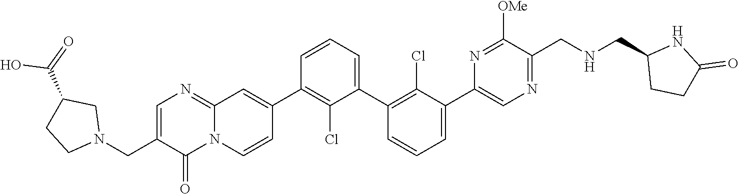 [SMILES: COc1nc(-c2cccc(-c3cccc(-c4ccn5c(=O)c(CN6CC[C@H](C(=O)O)C6)cnc5c4)c3Cl)c2Cl)cnc1CNC[C@@H]1CCC(=O)N1] | 0.0003 |
| | (S)-1-((8-(2,2′-dichloro-3′-(6-methoxy-5-(((((S)-5-oxopyrrolidin-2-yl)methyl)amino)methyl)pyrazin-2-yl)-[1,1′-biphenyl]-3- | |
| | yl)-4-oxo-4H-pyrido[1,2-a]pyrimidin-3-yl)methyl)pyrrolidine-3-carboxylic acid | |
| 117 | 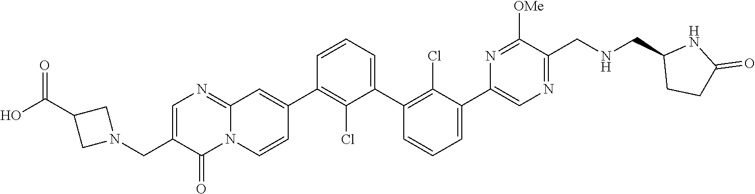 [SMILES: COc1nc(-c2cccc(-c3cccc(-c4ccn5c(=O)c(CN6CC(C(=O)O)C6)cnc5c4)c3Cl)c2Cl)cnc1CNC[C@@H]1CCC(=O)N1] | 0.0004 |
| | (S)-1-((8-(2,2′-dichloro-3′-(6-methoxy-5-((((5-oxopyrrolidin-2-yl)methyl)amino)methyl)pyrazin-2-yl)-[1,1′-biphenyl]-3-yl)-4- | |
| | oxo-4H-pyrido[1,2-a]pyrimidin-3-yl)methyl)azetidine-3-carboxylic acid | |
| 118 | 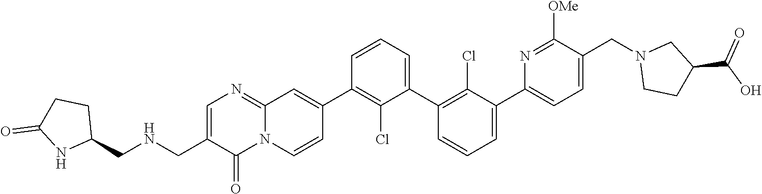 [SMILES: COc1nc(-c2cccc(-c3cccc(-c4ccn5c(=O)c(CNC[C@@H]6CCC(=O)N6)cnc5c4)c3Cl)c2Cl)ccc1CN1CC[C@H](C(=O)O)C1] | 0.0002 |
| | (S)-1-((6-(2,2′-dichloro-3′-(4-oxo-3-(((((S)-5-oxopyrrolidin-2-yl)methyl)amino)methyl)-4H-pyrido[1,2-a]pyrimidin-8-yl)-[1,1′- | |
| | biphenyl]-3-yl)-2-methoxypyridin-3-yl)methyl)pyrrolidine-3-carboxylic acid | |
| 119 | 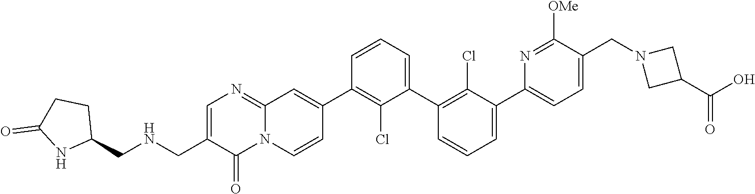 [SMILES: COc1nc(-c2cccc(-c3cccc(-c4ccn5c(=O)c(CNC[C@@H]6CCC(=O)N6)cnc5c4)c3Cl)c2Cl)ccc1CN1CC(C(=O)O)C1] | 0.0003 |
| | (S)-1-((6-(2,2′-dichloro-3′-(4-oxo-3-((((5-oxopyrrolidin-2-yl)methyl)amino)methyl)-4H-pyrido[1,2-a]pyrimidin-8-yl)-[1,1′- | |
| | biphenyl]-3-yl)-2-methoxypyridin-3-yl)methyl)azetidine-3-carboxylic acid | |
| 120 | 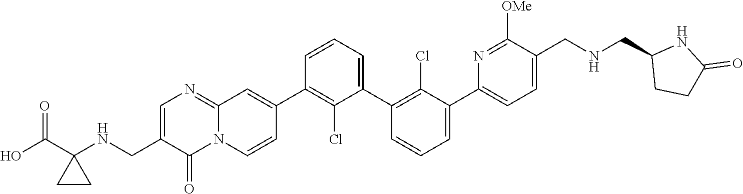 [SMILES: COc1nc(-c2cccc(-c3cccc(-c4ccn5c(=O)c(CNC6(C(=O)O)CC6)cnc5c4)c3Cl)c2Cl)ccc1CNC[C@@H]1CCC(=O)N1] | 0.0004 |
| | (S)-1-(((8-(2,2′-dichloro-3′-(6-methoxy-5-((((5-oxopyrrolidin-2-yl)methyl)amino)methyl)pyridin-2-yl)-[1,1′-biphenyl]-3-yl)-4- | |
| | oxo-4H-pyrido[1,2-a]pyrimidin-3-yl)methyl)amino)cyclopropane-1-carboxylic acid | |
| 121 | 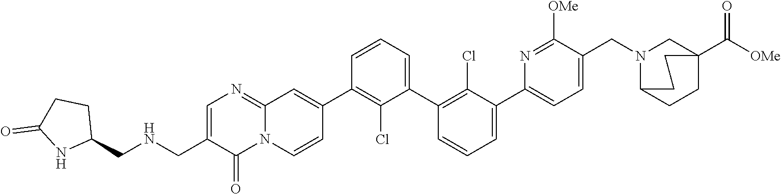 [SMILES: COC(=O)C12CCC(CC1)N(Cc1ccc(-c3cccc(-c4cccc(-c5ccn6c(=O)c(CNC[C@@H]7CCC(=O)N7)cnc6c5)c4Cl)c3Cl)nc1OC)C2] | 0.001 |
| | methyl (S)-2-((6-(2,2′-dichloro-3′-(4-oxo-3-((((5-oxopyrrolidin-2-yl)methyl)amino)methyl)-4H-pyrido[1,2-a]pyrimidin-8-yl)-[1,1′- | |
| | biphenyl]-3-yl)-2-methoxypyridin-3-yl)methyl)-2-azabicyclo[2.2.2]octane-4-carboxylate | |
| 122 | 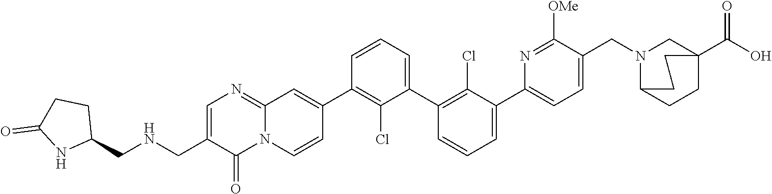 [SMILES: COc1nc(-c2cccc(-c3cccc(-c4ccn5c(=O)c(CNC[C@@H]6CCC(=O)N6)cnc5c4)c3Cl)c2Cl)ccc1CN1CC2(C(=O)O)CCC1CC2] | 0.0008 |
| | (S)-2-((6-(2,2′-dichloro-3′-(4-oxo-3-((((5-oxopyrrolidin-2-yl)methyl)amino)methyl)-4H-pyrido[1,2-a]pyrimidin-8-yl)-[1,1′- | |
| | biphenyl]-3-yl)-2-methoxypyridin-3-yl)methyl)-2-azabicyclo[2.2.2]octane-4-carboxylic acid | |
| 123 | 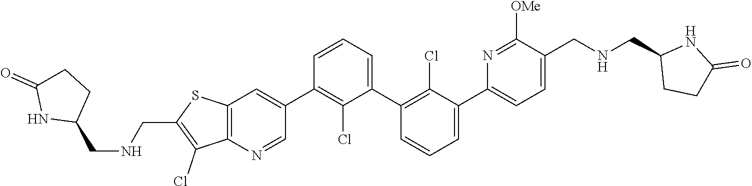 [SMILES: COc1nc(-c2cccc(-c3cccc(-c4cnc5c(Cl)c(CNC[C@@H]6CCC(=O)N6)sc5c4)c3Cl)c2Cl)ccc1CNC[C@@H]1CCC(=O)N1] | 0.0002 |
| | (S)-5-((((3-chloro-6-(2,2′-dichloro-3′-(6-methoxy-5-(((((S)-5-oxopyrrolidin-2-yl)methyl)amino)methyl)pyridin-2-yl)-[1,1′- | |
| | biphenyl]-3-yl)thieno[3,2-b]pyridin-2-yl)methyl)amino)methyl)pyrrolidin-2-one | |
| 124 | 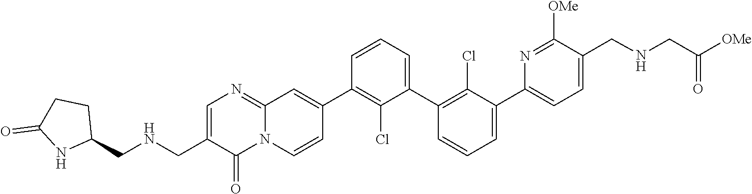 [SMILES: COC(=O)CNCc1ccc(-c2cccc(-c3cccc(-c4ccn5c(=O)c(CNC[C@@H]6CCC(=O)N6)cnc5c4)c3Cl)c2Cl)nc1OC] | 0.0038 |
| | methyl (S)-((6-(2,2′-dichloro-3′-(4-oxo-3-((((5-oxopyrrolidin-2-yl)methyl)amino)methyl)-4H-pyrido[1,2-a]pyrimidin-8-yl)- | |
| | [1,1′-biphenyl]-3-yl)-2-methoxypyridin-3-yl)methyl)glycinate | |
| 125 | 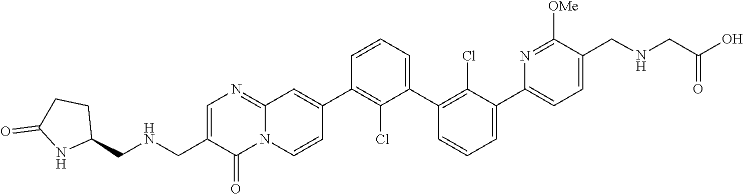 [SMILES: COc1nc(-c2cccc(-c3cccc(-c4ccn5c(=O)c(CNC[C@@H]6CCC(=O)N6)cnc5c4)c3Cl)c2Cl)ccc1CNCC(=O)O] | 0.0007 |
| | (S)-((6-(2,2′-dichloro-3′-(4-oxo-3-((((5-oxopyrrolidin-2-yl)methyl)amino)methyl)-4H-pyrido[1,2-a]pyrimidin-8-yl)-[1,1′- | |
| | biphenyl]-3-yl)-2-methoxypyridin-3-yl)methyl)glycine | |
| 126 | 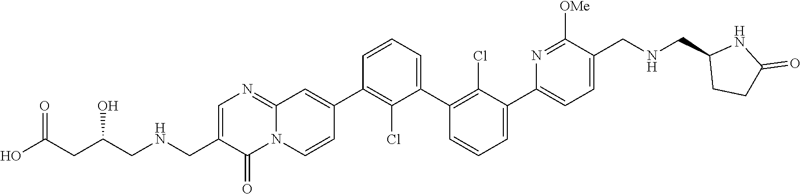 [SMILES: COc1nc(-c2cccc(-c3cccc(-c4ccn5c(=O)c(CNC[C@@H](O)CC(=O)O)cnc5c4)c3Cl)c2Cl)ccc1CNC[C@@H]1CCC(=O)N1] | 0.0004 |
| | (S)-4-(((8-(2,2′-dichloro-3′-(6-methoxy-5-(((((S)-5-oxopyrrolidin-2-yl)methyl)amino)methyl)pyridin-2-yl)-[1,1′-biphenyl]-3-yl)-4-oxo- | |
| | 4H-pyrido[1,2-a]pyrimidin-3-yl)methyl)amino)-3-hydroxybutanoic acid | |
| 127 | 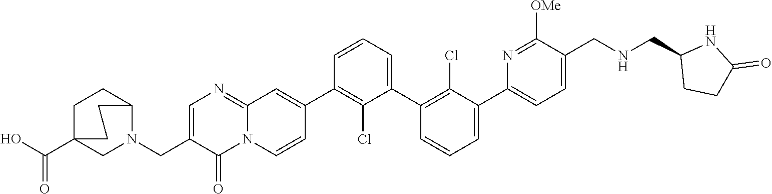 [SMILES: COc1nc(-c2cccc(-c3cccc(-c4ccn5c(=O)c(CN6CC7(C(=O)O)CCC6CC7)cnc5c4)c3Cl)c2Cl)ccc1CNC[C@@H]1CCC(=O)N1] | 0.0003 |
| | (S)-2-((8-(2,2′-dichloro-3′-(6-methoxy-5-((((5-oxopyrrolidin-2-yl)methyl)amino)methyl)pyridin-2-yl)-[1,1′-biphenyl]-3-yl)-4-oxo- | |
| | 4H-pyrido[1,2-a]pyrimidin-3-yl)methyl)-2-azabicyclo[2.2.2]octane-4-carboxylic acid | |
| 128 | 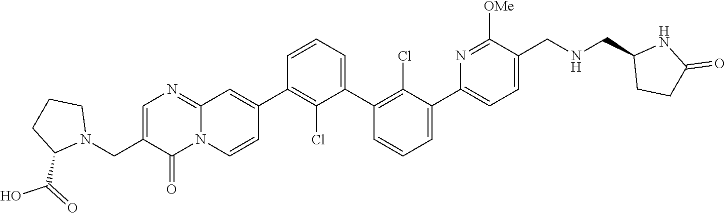 [SMILES: COc1nc(-c2cccc(-c3cccc(-c4ccn5c(=O)c(CN6CCC[C@H]6C(=O)O)cnc5c4)c3Cl)c2Cl)ccc1CNC[C@@H]1CCC(=O)N1] | 0.0002 |
| | ((8-(2,2′-dichloro-3′-(6-methoxy-5-(((((S)-5-oxopyrrolidin-2-yl)methyl)amino)methyl)pyridin-2-yl)-[1,1′-biphenyl]-3-yl)- | |
| | 4-oxo-4H-pyrido[1,2-a]pyrimidin-3-yl)methyl)-L-proline | |
| 129 | 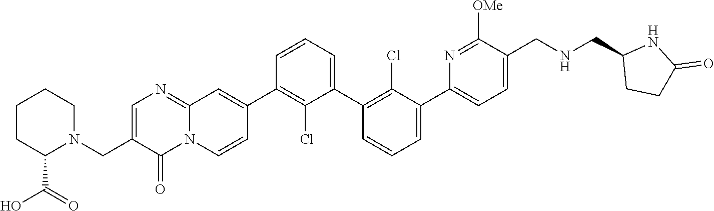 [SMILES: COc1nc(-c2cccc(-c3cccc(-c4ccn5c(=O)c(CN6CCCC[C@H]6C(=O)O)cnc5c4)c3Cl)c2Cl)ccc1CNC[C@@H]1CCC(=O)N1] | 0.0003 |
| | (S)-1-((8-(2,2′-dichloro-3′-(6-methoxy-5-(((((S)-5-oxopyrrolidin-2-yl)methyl)amino)methyl)pyridin-2-yl)-[1,1′-biphenyl]- | |
| | 3-yl)-4-oxo-4H-pyrido[1,2-a]pyrimidin-3-yl)methyl)piperidine-2-carboxylic acid | |
| 130 | 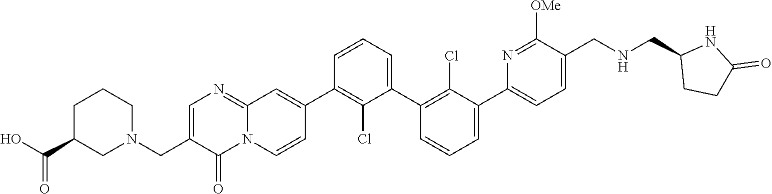 [SMILES: COc1nc(-c2cccc(-c3cccc(-c4ccn5c(=O)c(CN6CCC[C@H](C(=O)O)C6)cnc5c4)c3Cl)c2Cl)ccc1CNC[C@@H]1CCC(=O)N1] | 0.0003 |
| | (S)-1-((8-(2,2′-dichloro-3′-(6-methoxy-5-(((((S)-5-oxopyrrolidin-2-yl)methyl)amino)methyl)pyridin-2-yl)-[1,1′-biphenyl]-3-yl)-4- | |
| | oxo-4H-pyrido[1,2-a]pyrimidin-3-yl)methyl)piperidine-3-carboxylic acid | |
| 131 | 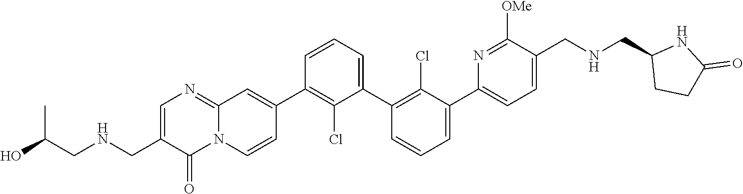 [SMILES: COc1nc(-c2cccc(-c3cccc(-c4ccn5c(=O)c(CNC[C@H](C)O)cnc5c4)c3Cl)c2Cl)ccc1CNC[C@@H]1CCC(=O)N1] | 0.0017 |
| | 8-(2,2′-dichloro-3′-(6-methoxy-5-(((((S)-5-oxopyrrolidin-2-yl)methyl)amino)methyl)pyridin-2-yl)-[1,1′-biphenyl]-3-yl)-3- | |
| | ((((S)-2-hydroxypropyl)amino)methyl)-4H-pyrido[1,2-a]pyrimidin-4-one | |
| 132 | 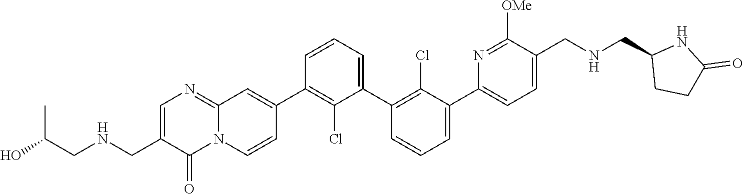 [SMILES: COc1nc(-c2cccc(-c3cccc(-c4ccn5c(=O)c(CNC[C@@H](C)O)cnc5c4)c3Cl)c2Cl)ccc1CNC[C@@H]1CCC(=O)N1] | 0.0017 |
| | 8-(2,2′-dichloro-3′-(6-methoxy-5-(((((S)-5-oxopyrrolidin-2-yl)methyl)amino)methyl)pyridin-2-yl)-[1,1′-biphenyl]-3-yl)-3- | |
| | ((((R)-2-hydroxypropyl)amino)methyl)-4H-pyrido[1,2-a]pyrimidin-4-one | |
| 133 | 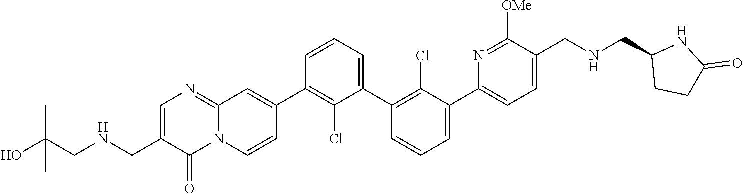 [SMILES: COc1nc(-c2cccc(-c3cccc(-c4ccn5c(=O)c(CNCC(C)(C)O)cnc5c4)c3Cl)c2Cl)ccc1CNC[C@@H]1CCC(=O)N1] | 0.0015 |
| | (S)-8-(2,2′-dichloro-3′-(6-methoxy-5-((((5-oxopyrrolidin-2-yl)methyl)amino)methyl)pyridin-2-yl)-[1,1′-biphenyl]-3-yl)-3- | |
| | (((2-hydroxy-2-methylpropyl)amino)methyl)-4H-pyrido[1,2-a]pyrimidin-4-one | |
| 134 | 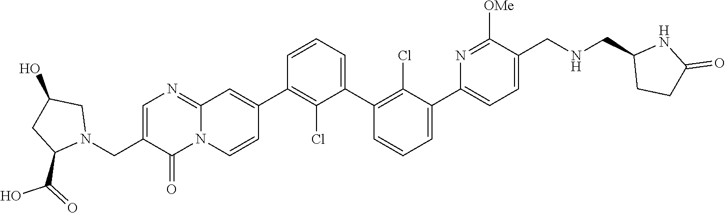 [SMILES: COc1nc(-c2cccc(-c3cccc(-c4ccn5c(=O)c(CN6C[C@H](O)C[C@@H]6C(=O)O)cnc5c4)c3Cl)c2Cl)ccc1CNC[C@@H]1CCC(=O)N1] | 0.0011 |
| | (2R,4R)-1-((8-(2,2′-dichloro-3′-(6-methoxy-5-(((((S)-5-oxopyrrolidin-2-yl)methyl)amino)methyl)pyridin-2-yl)-[1,1′- | |
| | biphenyl]-3-yl)-4-oxo-4H-pyrido[1,2-a]pyrimidin-3-yl)methyl)-4-hydroxypyrrolidine-2-carboxylic acid | |
| 135 | 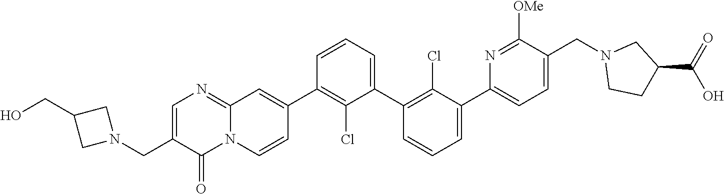 [SMILES: COc1nc(-c2cccc(-c3cccc(-c4ccn5c(=O)c(CN6CC(CO)C6)cnc5c4)c3Cl)c2Cl)ccc1CN1CC[C@H](C(=O)O)C1] | 0.0002 |
| | (S)-1-((6-(2,2′-dichloro-3′-(3-((3-(hydroxymethyl)azetidin-1-yl)methyl)-4-oxo-4H-pyrido[1,2-a]pyrimidin-8-yl)-[1,1′- | |
| | biphenyl]-3-yl)-2-methoxypyridin-3-yl)methyl)pyrrolidine-3-carboxylic acid | |
| 136 | 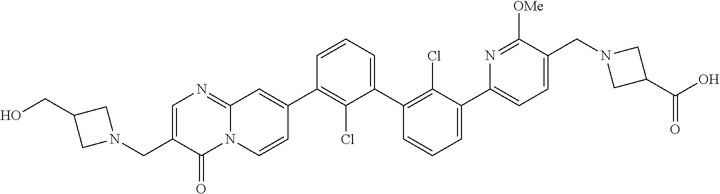 [SMILES: COc1nc(-c2cccc(-c3cccc(-c4ccn5c(=O)c(CN6CC(CO)C6)cnc5c4)c3Cl)c2Cl)ccc1CN1CC(C(=O)O)C1] | 0.0002 |
| | 1-((6-(2,2′-dichloro-3′-(3-((3-(hydroxymethyl)azetidin-1-yl)methyl)-4-oxo-4H-pyrido[1,2-a]pyrimidin-8-yl)-[1,1′- | |
| | biphenyl]-3-yl)-2-methoxypyridin-3-yl)methyl)azetidine-3-carboxylic acid | |
| 137 | 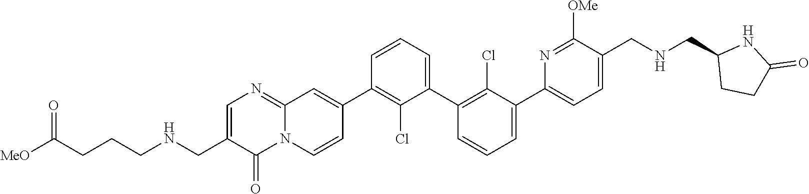 [SMILES: COC(=O)CCCNCc1cnc2cc(-c3cccc(-c4cccc(-c5ccc(CNC[C@@H]6CCC(=O)N6)c(OC)n5)c4Cl)c3Cl)ccn2c1=O] | 0.0017 |
| | methyl (S)-4-(((8-(2,2′-dichloro-3′-(6-methoxy-5-((((5-oxopyrrolidin-2-yl)methyl)amino)methyl)pyridin-2-yl)-[1,1′-biphenyl]-3-yl)-4- | |
| | oxo-4H-pyrido[1,2-a]pyrimidin-3-yl)methyl)amino)butanoate | |
| 138 | 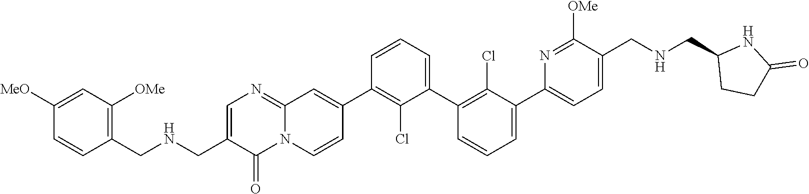 [SMILES: COc1ccc(CNCc2cnc3cc(-c4cccc(-c5cccc(-c6ccc(CNC[C@@H]7CCC(=O)N7)c(OC)n6)c5Cl)c4Cl)ccn3c2=O)c(OC)c1] | 0.0014 |
| | (S)-8-(2,2′-dichloro-3′-(6-methoxy-5-((((5-oxopyrrolidin-2-yl)methyl)amino)methyl)pyridin-2-yl)-[1,1′-biphenyl]-3-yl)-3-(((2,4- | |
| | dimethoxybenzyl)amino)methyl)-4H-pyrido[1,2-a]pyrimidin-4-one | |
| 139 | 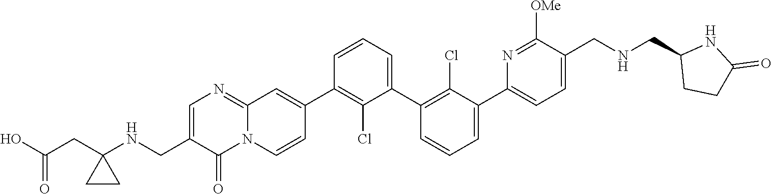 [SMILES: COc1nc(-c2cccc(-c3cccc(-c4ccn5c(=O)c(CNC6(CC(=O)O)CC6)cnc5c4)c3Cl)c2Cl)ccc1CNC[C@@H]1CCC(=O)N1] | 0.0004 |
| | (S)-2-(1-(((8-(2,2′-dichloro-3′-(6-methoxy-5-((((5-oxopyrrolidin-2-yl)methyl)amino)methyl)pyridin-2-yl)-[1,1′-biphenyl]-3-yl)-4- | |
| | oxo-4H-pyrido[1,2-a]pyrimidin-3-yl)methyl)amino)cyclopropyl)acetic acid | |
| 140 | 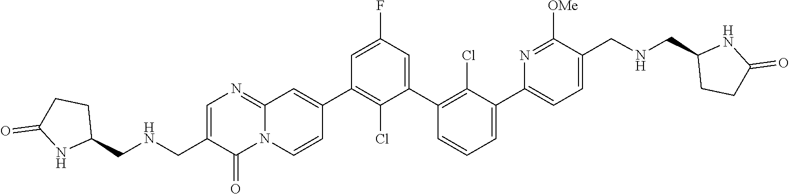 [SMILES: COc1nc(-c2cccc(-c3cc(F)cc(-c4ccn5c(=O)c(CNC[C@@H]6CCC(=O)N6)cnc5c4)c3Cl)c2Cl)ccc1CNC[C@@H]1CCC(=O)N1] | 0.002 |
| | 8-(2,2′-dichloro-5-fluoro-3′-(6-methoxy-5-(((((S)-5-oxopyrrolidin-2-yl)methyl)amino)methyl)pyridin-2-yl)-[1,1′-biphenyl]-3-yl)-3- | |
| | (((((S)-5-oxopyrrolidin-2-yl)methyl)amino)methyl)-4H-pyrido[1,2-a]pyrimidin-4-one | |
| 141 | 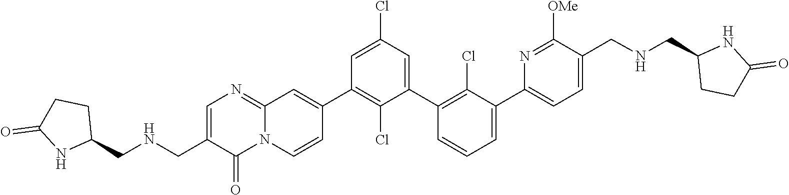 [SMILES: COc1nc(-c2cccc(-c3cc(Cl)cc(-c4ccn5c(=O)c(CNC[C@@H]6CCC(=O)N6)cnc5c4)c3Cl)c2Cl)ccc1CNC[C@@H]1CCC(=O)N1] | 0.0058 |
| | 3-(((((S)-5-oxopyrrolidin-2-yl)methyl)amino)methyl)-8-(2,2′,5-trichloro-3′-(6-methoxy-5-(((((S)-5-oxopyrrolidin-2- | |
| | yl)methyl)amino)methyl)pyridin-2-yl)-[1,1′-biphenyl]-3-yl)-4H-pyrido[1,2-a]pyrimidin-4-one | |
| 142 | 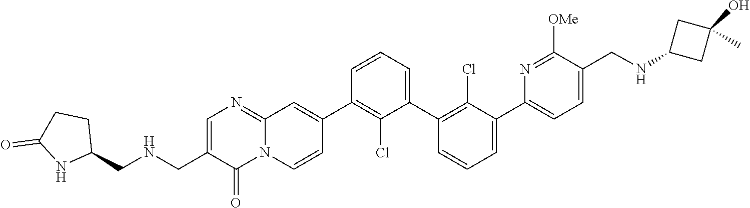 [SMILES: COc1nc(-c2cccc(-c3cccc(-c4ccn5c(=O)c(CNC[C@@H]6CCC(=O)N6)cnc5c4)c3Cl)c2Cl)ccc1CN[C@H]1C[C@](C)(O)C1] | 0.0017 |
| | 8-(2,2′-dichloro-3′-(5-((((1r,3r)-3-hydroxy-3-methylcyclobutyl)amino)methyl)-6-methoxypyridin-2-yl)-[1,1′-biphenyl]-3-yl)-3- | |
| | (((((S)-5-oxopyrrolidin-2-yl)methyl)amino)methyl)-4H-pyrido[1,2-a]pyrimidin-4-one | |
| 143 | 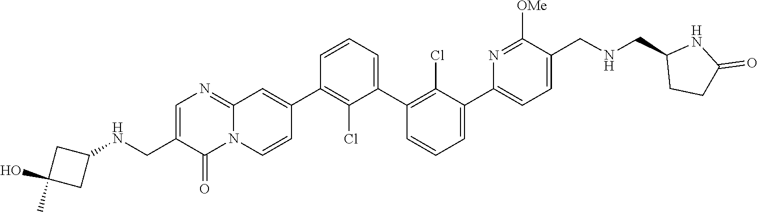 | 0.0018 |
| | 8-(2,2′-dichloro-3′-(6-methoxy-5-(((((S)-5-oxopyrrolidin-2-yl)methyl)amino)methyl)pyridin-2-yl)-[1,1′-biphenyl]-3-yl)-3- | |
| | ((((1r,3r)-3-hydroxy-3-methylcyclobutyl)amino)methyl)-4H-pyrido[1,2-a]pyrimidin-4-one | |
| 144 | 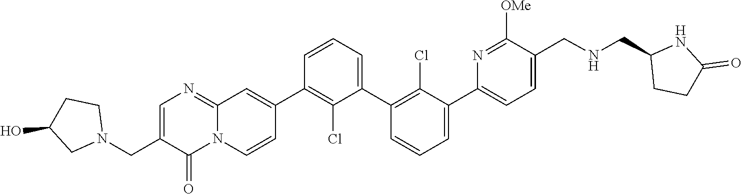 [SMILES: COc1nc(-c2cccc(-c3cccc(-c4ccn5c(=O)c(CN6CC[C@H](O)C6)cnc5c4)c3Cl)c2Cl)ccc1CNC[C@@H]1CCC(=O)N1] | 0.0031 |
| | 8-(2,2′-dichloro-3′-(6-methoxy-5-(((((S)-5-oxopyrrolidin-2-yl)methyl)amino)methyl)pyridin-2-yl)-[1,1′-biphenyl]-3-yl)-3-(((S)- | |
| | 3-hydroxypyrrolidin-1-yl)methyl)-4H-pyrido[1,2-a]pyrimidin-4-one | |
| 145 | 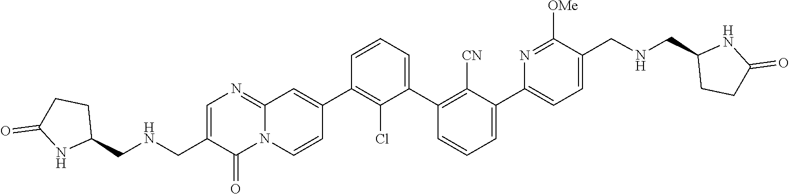 [SMILES: COc1nc(-c2cccc(-c3cccc(-c4ccn5c(=O)c(CNC[C@@H]6CCC(=O)N6)cnc5c4)c3Cl)c2C#N)ccc1CNC[C@@H]1CCC(=O)N1] | 0.0022 |
| | 2′-chloro-3-(6-methoxy-5-(((((S)-5-oxopyrrolidin-2-yl)methyl)amino)methyl)pyridin-2-yl)-3′-(4-oxo-3-(((((S)-5-oxopyrrolidin-2- | |
| | yl)methyl)amino)methyl)-4H-pyrido[1,2-a]pyrimidin-8-yl)-[1,1′-biphenyl]-2-carbonitrile | |
| 146 | 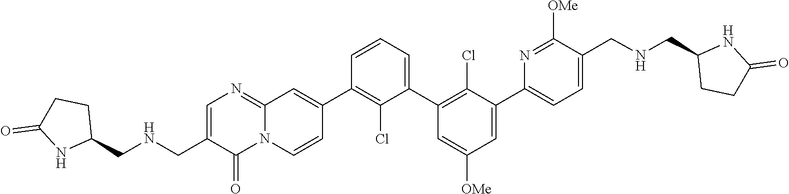 [SMILES: COc1cc(-c2ccc(CNC[C@@H]3CCC(=O)N3)c(OC)n2)c(Cl)c(-c2cccc(-c3ccn4c(=O)c(CNC[C@@H]5CCC(=O)N5)cnc4c3)c2Cl)c1] | 0.24 |
| | 8-(2,2′-dichloro-5′-methoxy-3′-(6-methoxy-5-(((((S)-5-oxopyrrolidin-2-yl)methyl)amino)methyl)pyridin-2-yl)-[1,1′-phenyl]-3-yl)-3- | |
| | (((((S)-5-oxopyrrolidin-2-yl)methyl)amino)methyl)-4H-pyrido[1,2-a]pyrimidin-4-one | |
| 147 | 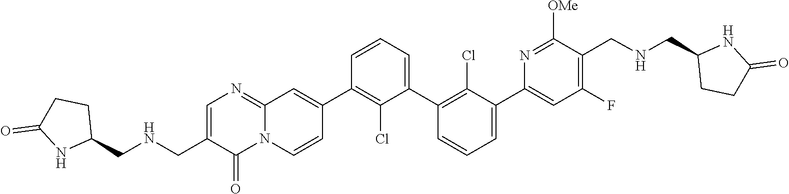 [SMILES: COc1nc(-c2cccc(-c3cccc(-c4ccn5c(=O)c(CNC[C@@H]6CCC(=O)N6)cnc5c4)c3Cl)c2Cl)cc(F)c1CNC[C@@H]1CCC(=O)N1] | 0.0039 |
| | 8-(2,2′-dichloro-3′-(4-fluoro-6-methoxy-5-(((((S)-5-oxopyrrolidin-2-yl)methyl)amino)methyl)pyridin-2-yl)-[1,1′-phenyl]-3-yl)-3- | |
| | (((((S)-5-oxopyrrolidin-2-yl)methyl)amino)methyl)-4H-pyrido[1,2-a]pyrimidin-4-one | |
| 148 | 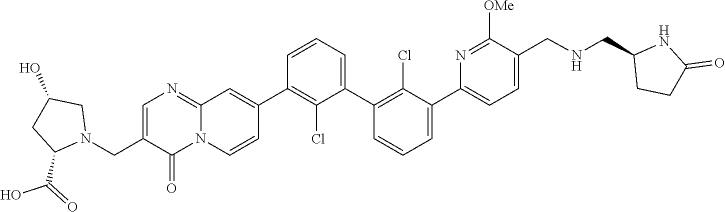 [SMILES: COc1nc(-c2cccc(-c3cccc(-c4ccn5c(=O)c(CN6C[C@@H](O)C[C@H]6C(=O)O)cnc5c4)c3Cl)c2Cl)ccc1CNC[C@@H]1CCC(=O)N1] | 0.0004 |
| | (2S,4S)-1-((8-(2,2′-dichloro-3′-(6-methoxy-5-(((((S)-5-oxopyrrolidin-2-yl)methyl)amino)methyl)pyridin-2-yl)-[1,1′- | |
| | biphenyl]-3-yl)-4-oxo-4H-pyrido[1,2-a]pyrimidin-3-yl)methyl)-4-hydroxypyrrolidine-2-carboxylic acid | |
| 149 | 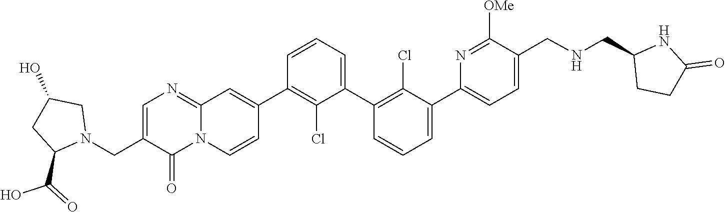 [SMILES: COc1nc(-c2cccc(-c3cccc(-c4ccn5c(=O)c(CN6C[C@@H](O)C[C@@H]6C(=O)O)cnc5c4)c3Cl)c2Cl)ccc1CNC[C@@H]1CCC(=O)N1] | 0.0003 |
| | (2R,4S)-1-((8-(2,2′-dichloro-3′-(6-methoxy-5-(((((S)-5-oxopyrrolidin-2-yl)methyl)amino)methyl)pyridin-2-yl)-[1,1′- | |
| | biphenyl]-3-yl)-4-oxo-4H-pyrido[1,2-a]pyrimidin-3-yl)methyl)-4-hydroxypyrrolidine-2-carboxylic acid | |
| 150 | 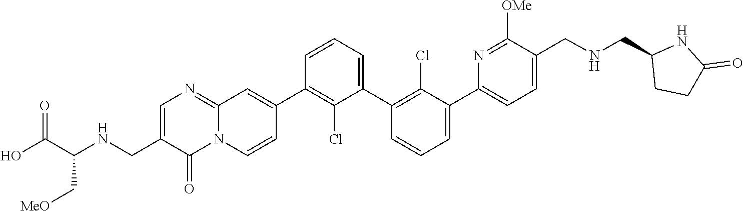 [SMILES: COC[C@@H](NCc1cnc2cc(-c3cccc(-c4cccc(-c5ccc(CNC[C@@H]6CCC(=O)N6)c(OC)n5)c4Cl)c3Cl)ccn2c1=O)C(=O)O] | 0.0003 |
| | N-((8-(2,2′-dichloro-3′-(6-methoxy-5-(((((S)-5-oxopyrrolidin-2-yl)methyl)amino)methyl)pyridin-2-yl)-[1,1′-biphenyl]-3-yl)-4- | |
| | oxo-4H-pyrido[1,2-a]pyrimidin-3-yl)methyl)-O-methyl-D-serine | |
| 151 | 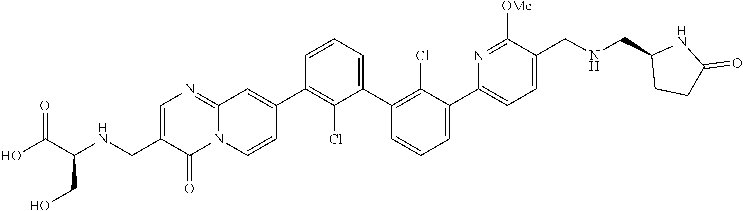 [SMILES: COc1nc(-c2cccc(-c3cccc(-c4ccn5c(=O)c(CN[C@@H](CO)C(=O)O)cnc5c4)c3Cl)c2Cl)ccc1CNC[C@@H]1CCC(=O)N1] | 0.0003 |
| | ((8-(2,2′-dichloro-3′-(6-methoxy-5-(((((S)-5-oxopyrrolidin-2-yl)methyl)amino)methyl)pyridin-2-yl)-[1,1′-biphenyl]-3-yl)-4- | |
| | oxo-4H-pyrido[1,2-a]pyrimidin-3-yl)methyl)-L-serine | |
| 152 | 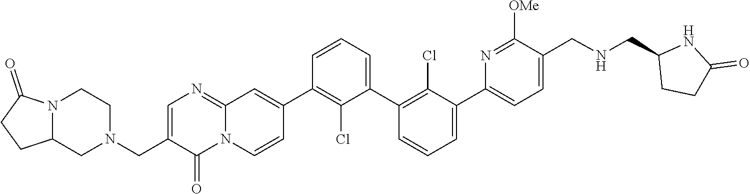 [SMILES: COc1nc(-c2cccc(-c3cccc(-c4ccn5c(=O)c(CN6CCN7C(=O)CCC7C6)cnc5c4)c3Cl)c2Cl)ccc1CNC[C@@H]1CCC(=O)N1] | 0.0031 |
| | 8-(2,2′-dichloro-3′-(6-methoxy-5-(((((S)-5-oxopyrrolidin-2-yl)methyl)amino)methyl)pyridin-2-yl)-[1,1′-biphenyl]-3-yl)-3-((6- | |
| | oxohexahydropyrrolo[1,2-a]pyrazin-2(1H)-yl)methyl)-4H-pyrido[1,2-a]pyrimidin-4-one | |
| | mixture of 2 diastereoisomers | |
| 153 | 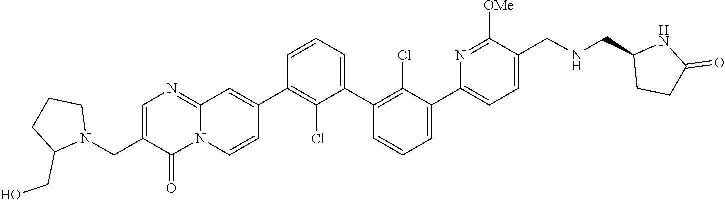 [SMILES: COc1nc(-c2cccc(-c3cccc(-c4ccn5c(=O)c(CN6CCCC6CO)cnc5c4)c3Cl)c2Cl)ccc1CNC[C@@H]1CCC(=O)N1] | 0.0008 |
| | 8-(2,2′-dichloro-3′-(6-methoxy-5-(((((S)-5-oxopyrrolidin-2-yl)methyl)amino)methyl)pyridin-2-yl)-[1,1′-biphenyl]-3-yl)-3- | |
| | ((2-(hydroxymethyl)pyrrolidin-1-yl)methyl)-4H-pyrido[1,2-a]pyrimidin-4-one | |
| | mixture of 2 diastereoisomers | |
| 154 | 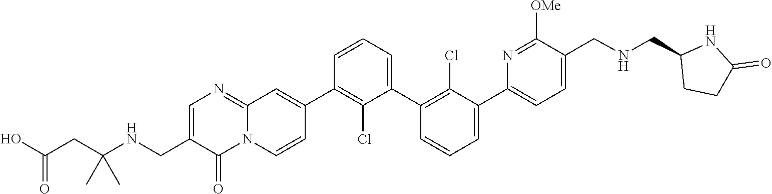 [SMILES: COc1nc(-c2cccc(-c3cccc(-c4ccn5c(=O)c(CNC(C)(C)CC(=O)O)cnc5c4)c3Cl)c2Cl)ccc1CNC[C@@H]1CCC(=O)N1] | 0.0004 |
| | (S)-3-(((8-(2,2′-dichloro-3′-(6-methoxy-5-((((5-oxopyrrolidin-2-yl)methyl)amino)methyl)pyridin-2-yl)-[1,1′-biphenyl]-3-yl)-4-oxo- | |
| | 4H-pyrido[1,2-a]pyrimidin-3-yl)methyl)amino)-3-methylbutanoic acid | |
| 155 | 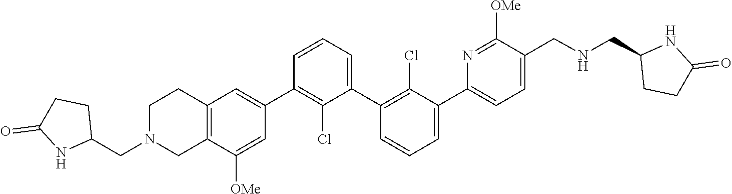 [SMILES: COc1cc(-c2cccc(-c3cccc(-c4ccc(CNC[C@@H]5CCC(=O)N5)c(OC)n4)c3Cl)c2Cl)cc2c1CN(CC1CCC(=O)N1)CC2] | 0.0004 |
| | 5-((6-(2,2′-dichloro-3′-(6-methoxy-5-(((((S)-5-oxopyrrolidin-2-yl)methyl)amino)methyl)pyridin-2-yl)-[1,1′-biphenyl]-3-yl)-8- | |
| | methoxy-3,4-dihydroisoquinolin-2(1H)-yl)methyl)pyrrolidin-2-one | |
| 156 | 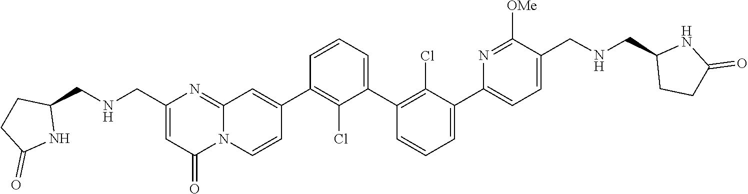 [SMILES: COc1nc(-c2cccc(-c3cccc(-c4ccn5c(=O)cc(CNC[C@@H]6CCC(=O)N6)nc5c4)c3Cl)c2Cl)ccc1CNC[C@@H]1CCC(=O)N1] | 0.0014 |
| | 8-(2,2′-dichloro-3′-(6-methoxy-5-(((((S)-5-oxopyrrolidin-2-yl)methyl)amino)methyl)pyridin-2-yl)-[1,1′-biphenyl]-3-yl)-2- | |
| | (((((S)-5-oxopyrrolidin-2-yl)methyl)amino)methyl)-4H-pyrido[1,2-a]pyrimidin-4-one | |
| 157 | 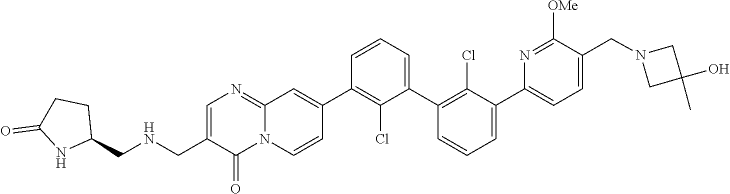 [SMILES: COc1nc(-c2cccc(-c3cccc(-c4ccn5c(=O)c(CNC[C@@H]6CCC(=O)N6)cnc5c4)c3Cl)c2Cl)ccc1CN1CC(C)(O)C1] | 0.0016 |
| | (S)-8-(2,2′-dichloro-3′-(5-((3-hydroxy-3-methylazetidin-1-yl)methyl)-6-methoxypyridin-2-yl)-[1,1′-biphenyl]-3-((((5- | |
| | oxopyrrolidin-2-yl)methyl)amino)methyl)-4H-pyrido[1,2-a]pyrimidin-4-one | |
| 158 | 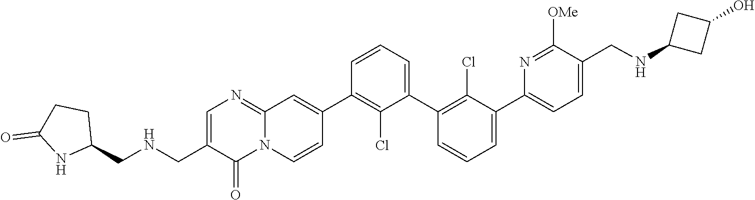 [SMILES: COc1nc(-c2cccc(-c3cccc(-c4ccn5c(=O)c(CNC[C@@H]6CCC(=O)N6)cnc5c4)c3Cl)c2Cl)ccc1CN[C@H]1C[C@H](O)C1] | 0.0020 |
| | 8-(2,2′-dichloro-3′-(5-((((1r,3r)-3-hydroxycyclobutyl)amino)methyl)-6-methoxypyridin-2-yl)-[1,1′-biphenyl]-3-yl)-3-(((((S)-5- | |
| | oxopyrrolidin-2-yl)methyl)amino)methyl)-4H-pyrido[1,2-a]pyrimidin-4-one | |
| 159 | 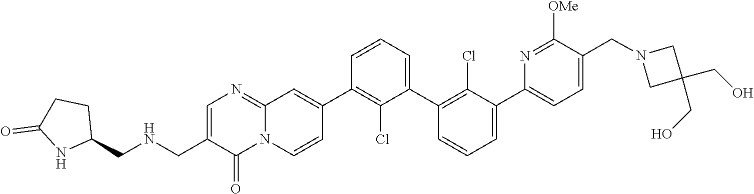 [SMILES: COc1nc(-c2cccc(-c3cccc(-c4ccn5c(=O)c(CNC[C@@H]6CCC(=O)N6)cnc5c4)c3Cl)c2Cl)ccc1CN1CC(CO)(CO)C1] | 0.0013 |
| | (S)-8-(3′-(5-((3,3-bis(hydroxymethyl)azetidin-1-yl)methyl)-6-methoxypyridin-2-yl)-2,2′-dichloro-[1,1′-biphenyl]-3-yl)-3-((((5- | |
| | oxopyrrolidin-2-yl)methyl)amino)methyl)-4H-pyrido[1,2-a]pyrimidin-4-one | |
| 160 | 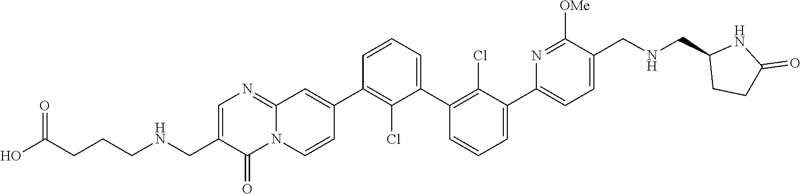 [SMILES: COc1nc(-c2cccc(-c3cccc(-c4ccn5c(=O)c(CNCCCC(=O)O)cnc5c4)c3Cl)c2Cl)ccc1CNC[C@@H]1CCC(=O)N1] | 0.0007 |
| | (S)-4-(((8-(2,2′-dichloro-3′-(6-methoxy-5-((((5-oxopyrrolidin-2-yl)methyl)amino)methyl)pyridin-2-yl)-[1,1′-biphenyl]-3-yl)-4-oxo-4H- | |
| | pyrido[1,2-a]pyrimidin-3-yl)methyl)amino)butanoic acid | |
| 161 | 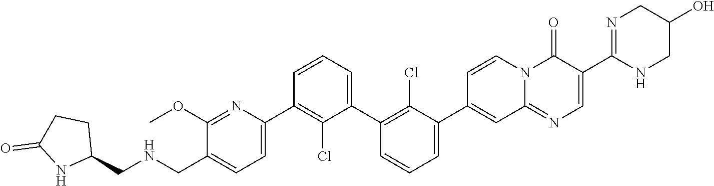 [SMILES: COc1nc(-c2cccc(-c3cccc(-c4ccn5c(=O)c(C6=NCC(O)CN6)cnc5c4)c3Cl)c2Cl)ccc1CNC[C@@H]1CCC(=O)N1] | 0.0001 |
| | 8-(2,2′-dichloro-3′-(6-methoxy-5-(((((S)-5-oxopyrrolidin-2-yl)methyl)amino)methyl)pyridin-2-yl)-[1,1′-biphenyl]-3-yl)-3- | |
| | (5-hydroxy-1,4,5,6-tetrahydropyrimidin-2-yl)-4H-pyrido[1,2-a]pyrimidin-4-one | |
| 162 | 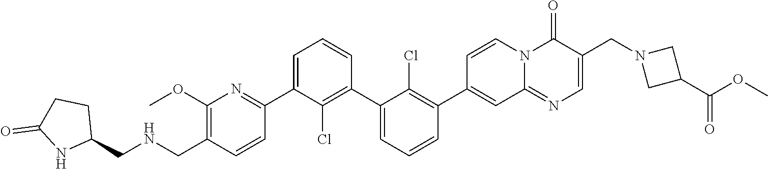 [SMILES: COC(=O)C1CN(Cc2cnc3cc(-c4cccc(-c5cccc(-c6ccc(CNC[C@@H]7CCC(=O)N7)c(OC)n6)c5Cl)c4Cl)ccn3c2=O)C1] | 0.0038 |
| | methyl (S)-1-((8-(2,2′-dichloro-3′-(6-methoxy-5-((((5-oxopyrrolidin-2-yl)methyl)amino)methyl)pyridin-2-yl)-[1,1′-biphenyl]-3-yl)- | |
| | 4-oxo-4H-pyrido[1,2-a]pyrimidin-3-yl)methyl)azetidine-3-carboxylate | |
| 163 | 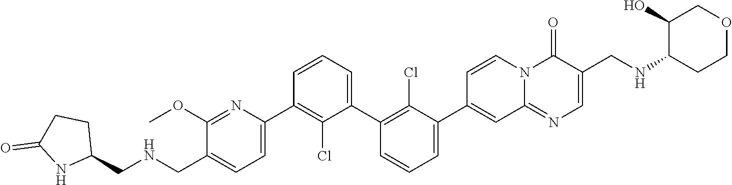 [SMILES: COc1nc(-c2cccc(-c3cccc(-c4ccn5c(=O)c(CN[C@H]6CCOC[C@@H]6O)cnc5c4)c3Cl)c2Cl)ccc1CNC[C@@H]1CCC(=O)N1] | 0.0008 |
| | 8-(2,2′-dichloro-3′-(6-methoxy-5-(((((S)-5-oxopyrrolidin-2-yl)methyl)amino)methyl)pyridin-2-yl)-[1,1′-biphenyl]-3-yl)-3- | |
| | ((((3R,4S)-3-hydroxytetrahydro-2H-pyran-4-yl)amino)methyl)-4H-pyrido[1,2-a]pyrimidin-4-one | |
| 164 | 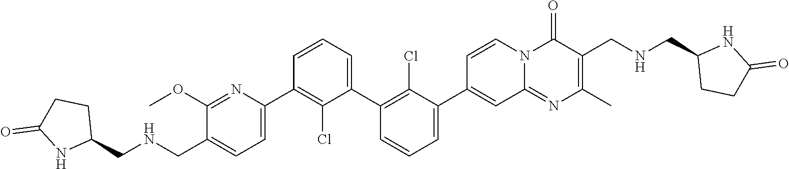 [SMILES: COc1nc(-c2cccc(-c3cccc(-c4ccn5c(=O)c(CNC[C@@H]6CCC(=O)N6)c(C)nc5c4)c3Cl)c2Cl)ccc1CNC[C@@H]1CCC(=O)N1] | 0.0013 |
| | 8-(2,2′-dichloro-3′-(6-methoxy-5-(((((S)-5-oxopyrrolidin-2-yl)methyl)amino)methyl)pyridin-2-yl)-[1,1′-biphenyl]-3-yl)-2-methyl-3- | |
| | (((((S)-5-oxopyrrolidin-2-yl)methyl)amino)methyl)-4H-pyrido[1,2-a]pyrimidin-4-one | |
| 165 | 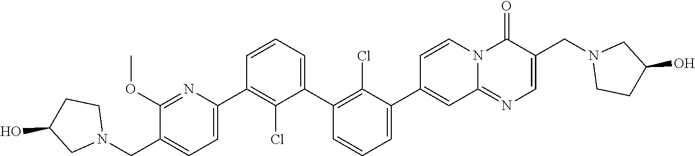 [SMILES: COc1nc(-c2cccc(-c3cccc(-c4ccn5c(=O)c(CN6CC[C@H](O)C6)cnc5c4)c3Cl)c2Cl)ccc1CN1CC[C@H](O)C1] | 0.0001 |
| | 8-(2,2′-dichloro-3′-(5-(((S)-3-hydroxypyrrolidin-1-yl)methyl)-6-methoxypyridin-2-yl)-[1,1′-biphenyl]-3-yl)-3-(((S)-3- | |
| | hydroxypyrrolidin-1-yl)methyl)-4H-pyrido[1,2-a]pyrimidin-4-one | |
| 166 | 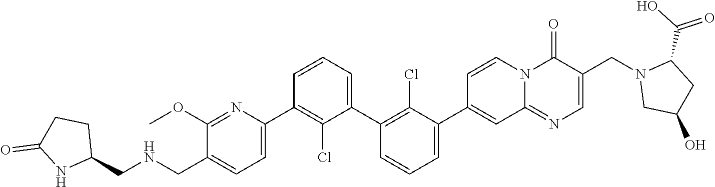 [SMILES: COc1nc(-c2cccc(-c3cccc(-c4ccn5c(=O)c(CN6C[C@H](O)C[C@H]6C(=O)O)cnc5c4)c3Cl)c2Cl)ccc1CNC[C@@H]1CCC(=O)N1] | 0.0001 |
| | (2S,4R)-1-((8-(2,2′-dichloro-3′-(6-methoxy-5-(((((S)-5-oxopyrrolidin-2-yl)methyl)amino)methyl)pyridin-2-yl)-[1,1′- | |
| | biphenyl]-3-yl)-4-oxo-4H-pyrido[1,2-a]pyrimidin-3-yl)methyl)-4-hydroxypyrrolidine-2-carboxylic acid | |
| 167 | 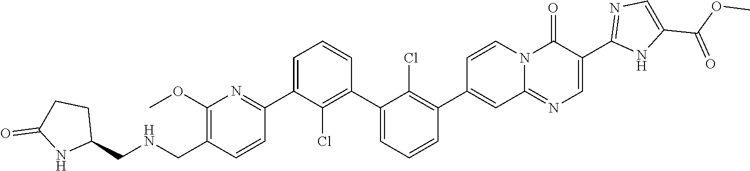 [SMILES: COC(=O)c1cnc(-c2cnc3cc(-c4cccc(-c5cccc(-c6ccc(CNC[C@@H]7CCC(=O)N7)c(OC)n6)c5Cl)c4Cl)ccn3c2=O)[nH]1] | 0.0005 |
| | methyl (S)-2-(8-(2,2′-dichloro-3′-(6-methoxy-5-((((5-oxopyrrolidin-2-yl)methyl)amino)methyl)pyridin-2-yl)-[1,1′-biphenyl]-3- | |
| | yl)-4-oxo-4H-pyrido[1,2-a]pyrimidin-3-yl)-1H-imidazole-5-carboxylate | |
| 168 | 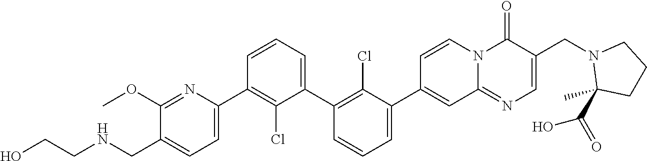 [SMILES: COc1nc(-c2cccc(-c3cccc(-c4ccn5c(=O)c(CN6CCC[C@@]6(C)C(=O)O)cnc5c4)c3Cl)c2Cl)ccc1CNCCO] | 0.0002 |
| | (S)-1-((8-(2,2′-dichloro-3′-(5-(((2-hydroxyethyl)amino)methyl)-6-methoxypyridin-2-yl)-[1,1′-biphenyl]-3-yl)- | |
| | 4-oxo-4H-pyrido[1,2-a]pyrimidin-3-yl)methyl)-2-methylpyrrolidine-2-carboxylic acid | |
| 169 | 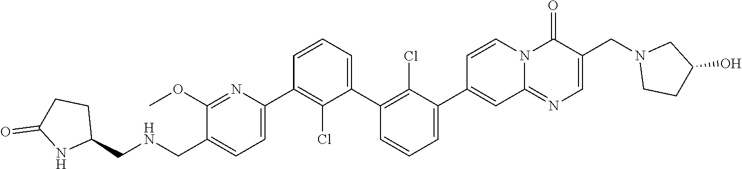 [SMILES: COc1nc(-c2cccc(-c3cccc(-c4ccn5c(=O)c(CN6CC[C@@H](O)C6)cnc5c4)c3Cl)c2Cl)ccc1CNC[C@@H]1CCC(=O)N1] | 0.0004 |
| | 8-(2,2′-dichloro-3′-(6-methoxy-5-(((((S)-5-oxopyrrolidin-2-yl)methyl)amino)methyl)pyridin-2-yl)-[1,1′-biphenyl]-3-yl)-3- | |
| | (((R)-3-hydroxypyrrolidin-1-yl)methyl)-4H-pyrido[1,2-a]pyrimidin-4-one | |
| 170 | 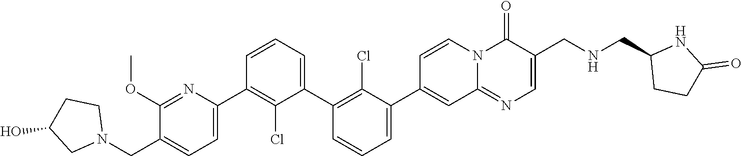 [SMILES: COc1nc(-c2cccc(-c3cccc(-c4ccn5c(=O)c(CNC[C@@H]6CCC(=O)N6)cnc5c4)c3Cl)c2Cl)ccc1CN1CC[C@@H](O)C1] | 0.0003 |
| | 8-(2,2′-dichloro-3′-(5-(((R)-3-hydroxypyrrolidin-1-yl)methyl)-6-methoxypyridin-2-yl)-[1,1′-biphenyl]-3-yl)-3-(((((S)-5- | |
| | oxopyrrolidin-2-yl)methyl)amino)methyl)-4H-pyrido[1,2-a]pyrimidin-4-one | |
| 171 | 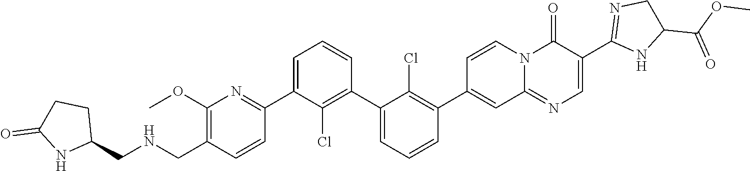 [SMILES: COC(=O)C1CN=C(c2cnc3cc(-c4cccc(-c5cccc(-c6ccc(CNC[C@@H]7CCC(=O)N7)c(OC)n6)c5Cl)c4Cl)ccn3c2=O)N1] | 0.0002 |
| | methyl 2-(8-(2,2′-dichloro-3′-(6-methoxy-5-(((((S)-5-oxopyrrolidin-2-yl)methyl)amino)methyl)pyridin-2-yl)-[1,1′-biphenyl]-3- | |
| | yl)-4-oxo-4H-pyrido[1,2-a]pyrimidin-3-yl)-4,5-dihydro-1H-imidazole-5-carboxylate | |
| | mixture of 2 diastereoisomers | |
| 172 | 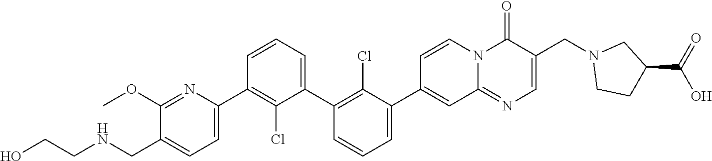 [SMILES: COc1nc(-c2cccc(-c3cccc(-c4ccn5c(=O)c(CN6CC[C@H](C(=O)O)C6)cnc5c4)c3Cl)c2Cl)ccc1CNCCO] | 0.0003 |
| | (S)-1-((8-(2,2′-dichloro-3′-(5-(((2-hydroxyethyl)amino)methyl)-6-methoxypyridin-2-yl)-[1,1′-biphenyl]-3-yl)-4-oxo-4H- | |
| | pyrido[1,2-a]pyrimidin-3-yl)methyl)pyrrolidine-3-carboxylic acid | |
| 173 | 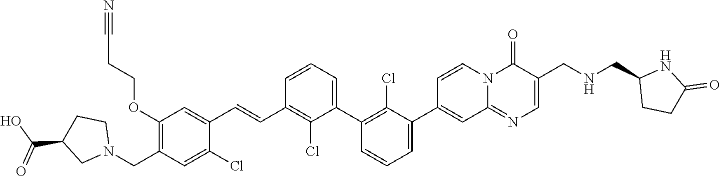 [SMILES: N#CCCOc1cc(/C=C/c2cccc(-c3cccc(-c4ccn5c(=O)c(CNC[C@@H]6CCC(=O)N6)cnc5c4)c3Cl)c2Cl)c(Cl)cc1CN1CC[C@H](C(=O)O)C1] | 0.0004 |
| | (S)-1-(5-chloro-2-(2-cyanoethoxy)-4-((E)-2-(2,2′-dichloro-3′-(4-oxo-3-(((((S)-5-oxopyrrolidin-2-yl)methyl)amino)methyl)- | |
| | 4H-pyrido[1,2-a]pyrimidin-8-yl)-[1,1′-biphenyl]-3-yl)vinyl)benzyl)pyrrolidine-3-carboxylic acid | |
| 174 | 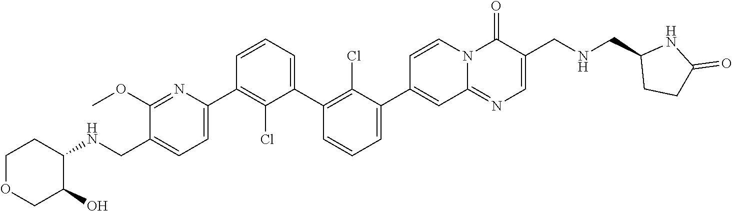 [SMILES: COc1nc(-c2cccc(-c3cccc(-c4ccn5c(=O)c(CNC[C@@H]6CCC(=O)N6)cnc5c4)c3Cl)c2Cl)ccc1CN[C@H]1CCOC[C@@H]1O] | 0.0008 |
| | 8-(2,2′-dichloro-3′-(5-((((3R,4S)-3-hydroxytetrahydro-2H-pyran-4-yl)amino)methyl)-6-methoxypyridin-2-yl)-[1,1′-biphenyl]- | |
| | 3-yl)-3-(((((S)-5-oxopyrrolidin-2-yl)methyl)amino)methyl)-4H-pyrido[1,2-a]pyrimidin-4-one | |
| 175 | 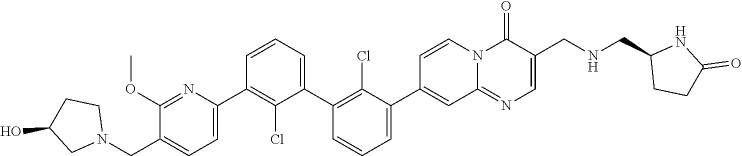 [SMILES: COc1nc(-c2cccc(-c3cccc(-c4ccn5c(=O)c(CNC[C@@H]6CCC(=O)N6)cnc5c4)c3Cl)c2Cl)ccc1CN1CC[C@H](O)C1] | 0.0001 |
| | 8-(2,2′-dichloro-3′-(5-(((S)-3-hydroxypyrrolidin-1-yl)methyl)-6-methoxypyridin-2-yl)-[1,1′-biphenyl]-3-yl)-3-(((((S)-5- | |
| | oxopyrrolidin-2-yl)methyl)amino)methyl)-4H-pyrido[1,2-a]pyrimidin-4-one | |
| 176 | 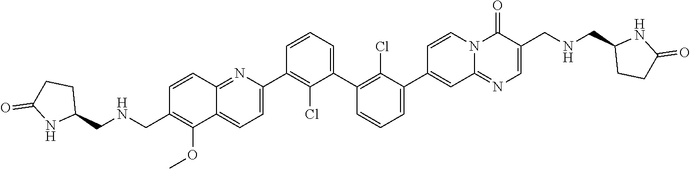 [SMILES: COc1c(CNC[C@@H]2CCC(=O)N2)ccc2nc(-c3cccc(-c4cccc(-c5ccn6c(=O)c(CNC[C@@H]7CCC(=O)N7)cnc6c5)c4Cl)c3Cl)ccc12] | 0.25 |
| | 8-(2,2′-dichloro-3′-(5-methoxy-6-(((((S)-5-oxopyrrolidin-2-yl)methyl)amino)methyl)quinolin-2-yl)-[1,1′-biphenyl]-3- | |
| | yl)-3-(((((S)-5-oxopyrrolidin-2-yl)methyl)amino)methyl)-4H-pyrido[1,2-a]pyrimidin-4-one | |
| 177 | 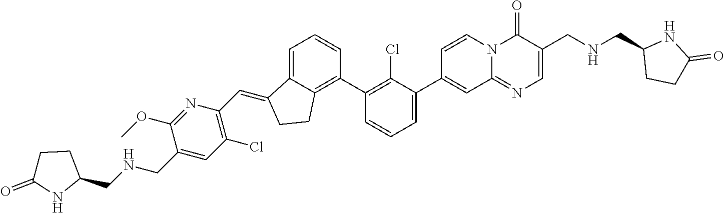 [SMILES: COc1nc(/C=C2\CCc3c2cccc3-c2cccc(-c3ccn4c(=O)c(CNC[C@@H]5CCC(=O)N5)cnc4c3)c2Cl)c(Cl)cc1CNC[C@@H]1CCC(=O)N1] | 0.0050 |
| | 8-(2-chloro-3-((E)-1-((3-chloro-6-methoxy-5-(((((S)-5-oxopyrrolidin-2-yl)methyl)amino)methyl)pyridin-2-yl)methylene)- | |
| | 2,3-dihydro-1H-inden-4-yl)phenyl)-3-(((((S)-5-oxopyrrolidin-2-yl)methyl)amino)methyl)-4H-pyrido[1,2-a]pyrimidin-4-one | |
| 178 | 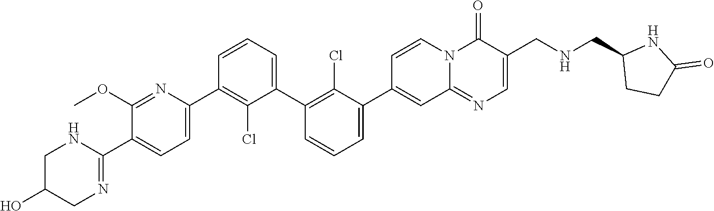 [SMILES: COc1nc(-c2cccc(-c3cccc(-c4ccn5c(=O)c(CNC[C@@H]6CCC(=O)N6)cnc5c4)c3Cl)c2Cl)ccc1C1=NCC(O)CN1] | 0.0035 |
| | 8-(2,2′-dichloro-3′-(5-(5-hydroxy-1,4,5,6-tetrahydropyrimidin-2-yl)-6-methoxypyridin-2-yl)-[1,1′-phenyl]-3-yl)-3-(((((S)- | |
| | 5-oxopyrrolidin-2-yl)methyl)amino)methyl)-4H-pyrido[1,2-a]pyrimidin-4-one | |
| 179 | 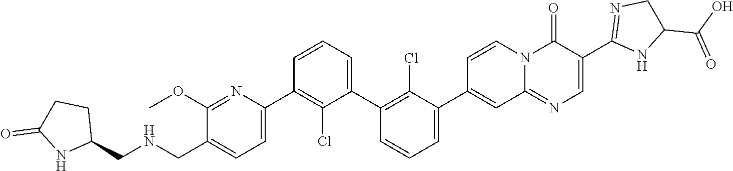 [SMILES: COc1nc(-c2cccc(-c3cccc(-c4ccn5c(=O)c(C6=NCC(C(=O)O)N6)cnc5c4)c3Cl)c2Cl)ccc1CNC[C@@H]1CCC(=O)N1] | 0.0001 |
| | 2-(8-(2,2′-dichloro-3′-(6-methoxy-5-(((((S)-5-oxopyrrolidin-2-yl)methyl)amino)methyl)pyridin-2-yl)-[1,1′-biphenyl]-3-yl)-4- | |
| | oxo-4H-pyrido[1,2-a]pyrimidin-3-yl)-4,5-dihydro-1H-imidazole-5-carboxylic acid | |
| | mixture of 2 diastereoisomers | |
| 180 | 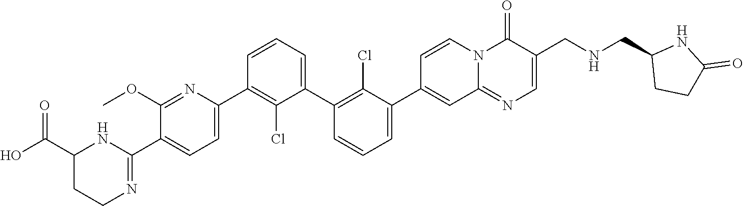 [SMILES: COc1nc(-c2cccc(-c3cccc(-c4ccn5c(=O)c(CNC[C@@H]6CCC(=O)N6)cnc5c4)c3Cl)c2Cl)ccc1C1=NCCC(C(=O)O)N1] | 0.0009 |
| | 2-(6-(2,2′-dichloro-3′-(4-oxo-3-(((((S)-5-oxopyrrolidin-2-yl)methyl)amino)methyl)-4H-pyrido[1,2-a]pyrimidin-8-yl)-[1,1′- | |
| | biphenyl]-3-yl)-2-methoxypyridin-3-yl)-3,4,5,6-tetrahydropyrimidine-4-carboxylic acid | |
| | mixture of 2 diastereoisomers | |
| 181 | 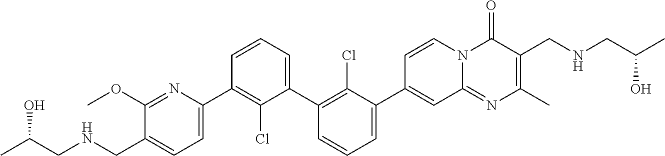 [SMILES: COc1nc(-c2cccc(-c3cccc(-c4ccn5c(=O)c(CNC[C@H](C)O)c(C)nc5c4)c3Cl)c2Cl)ccc1CNC[C@H](C)O] | 0.0038 |
| | 8-(2,2′-dichloro-3′-(5-((((S)-2-hydroxypropyl)amino)methyl)-6-methoxypyridin-2-yl)-[1,1′-biphenyl]-3-yl)-3- | |
| | ((((S)-2-hydroxypropyl)amino)methyl)-2-methyl-4H-pyrido[1,2-a]pyrimidin-4-one | |
| 182 | 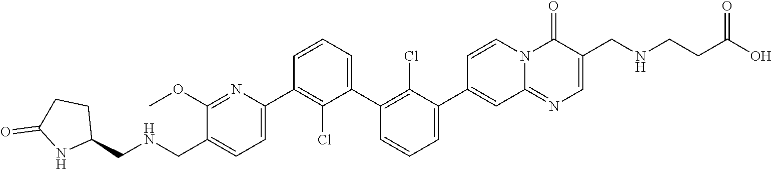 [SMILES: COc1nc(-c2cccc(-c3cccc(-c4ccn5c(=O)c(CNCCC(=O)O)cnc5c4)c3Cl)c2Cl)ccc1CNC[C@@H]1CCC(=O)N1] | 0.0001 |
| | (S)-3-(((8-(2,2′-dichloro-3′-(6-methoxy-5-((((5-oxopyrrolidin-2-yl)methyl)amino)methyl)pyridin-2-yl)-[1,1′-biphenyl]-3-yl)-4-oxo- | |
| | 4H-pyrido[1,2-a]pyrimidin-3-yl)methyl)amino)propanoic acid | |
| 183 | 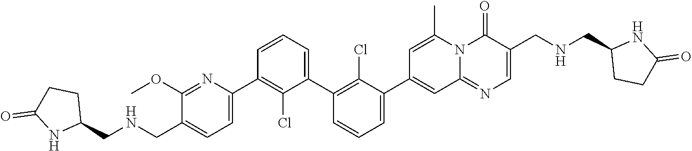 [SMILES: COc1nc(-c2cccc(-c3cccc(-c4cc(C)n5c(=O)c(CNC[C@@H]6CCC(=O)N6)cnc5c4)c3Cl)c2Cl)ccc1CNC[C@@H]1CCC(=O)N1] | 0.044 |
| | 8-(2,2′-dichloro-3′-(6-methoxy-5-(((((S)-5-oxopyrrolidin-2-yl)methyl)amino)methyl)pyridin-2-yl)-[1,1′-biphenyl]-3-yl)-6-methyl-3- | |
| | (((((S)-5-oxopyrrolidin-2-yl)methyl)amino)methyl)-4H-pyrido[1,2-a]pyrimidin-4-one | |
| 184 | 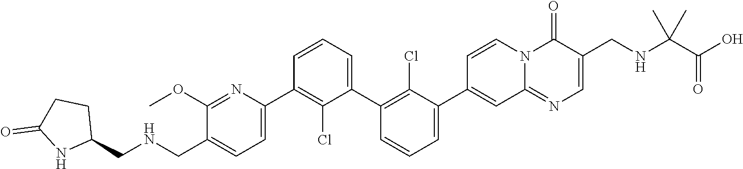 [SMILES: COc1nc(-c2cccc(-c3cccc(-c4ccn5c(=O)c(CNC(C)(C)C(=O)O)cnc5c4)c3Cl)c2Cl)ccc1CNC[C@@H]1CCC(=O)N1] | 0.0001 |
| | (S)-2-(((8-(2,2′-dichloro-3′-(6-methoxy-5-((((5-oxopyrrolidin-2-yl)methyl)amino)methyl)pyridin-2-yl)-[1,1′-biphenyl]-3-yl)-4- | |
| | oxo-4H-pyrido[1,2-a]pyrimidin-3-yl)methyl)amino)-2-methylpropanoic acid | |
| 185 | 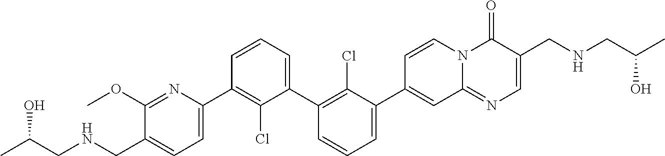 [SMILES: COc1nc(-c2cccc(-c3cccc(-c4ccn5c(=O)c(CNC[C@H](C)O)cnc5c4)c3Cl)c2Cl)ccc1CNC[C@H](C)O] | 0.0012 |
| | 8-(2,2′-dichloro-3′-(5-((((S)-2-hydroxypropyl)amino)methyl)-6-methoxypyridin-2-yl)-[1,1′-biphenyl]-3-yl)-3- | |
| | ((((S)-2-hydroxypropyl)amino)methyl)-4H-pyrido[1,2-a]pyrimidin-4-one | |
| 186 | 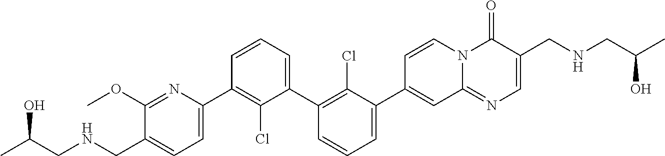 [SMILES: COc1nc(-c2cccc(-c3cccc(-c4ccn5c(=O)c(CNC[C@@H](C)O)cnc5c4)c3Cl)c2Cl)ccc1CNC[C@@H](C)O] | 0.0064 |
| | 8-(2,2′-dichloro-3′-(5-((((R)-2-hydroxypropyl)amino)methyl)-6-methoxypyridin-2-yl)-[1,1′-biphenyl]-3-yl)-3- | |
| | ((((R)-2-hydroxypropyl)amino)methyl)-4H-pyrido[1,2-a]pyrimidin-4-one | |
| 187 | 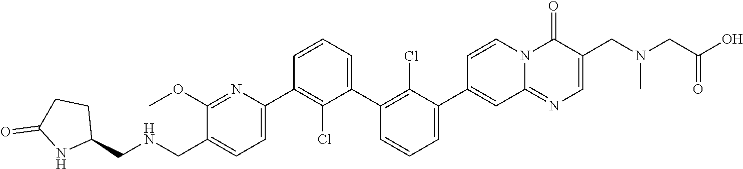 [SMILES: COc1nc(-c2cccc(-c3cccc(-c4ccn5c(=O)c(CN(C)CC(=O)O)cnc5c4)c3Cl)c2Cl)ccc1CNC[C@@H]1CCC(=O)N1] | 0.0001 |
| | (S)-N-((8-(2,2′-dichloro-3′-(6-methoxy-5-((((5-oxopyrrolidin-2-yl)methyl)amino)methyl)pyridin-2-yl)-[1,1′-biphenyl]-3-yl)-4- | |
| | oxo-4H-pyrido[1,2-a]pyrimidin-3-yl)methyl)-N-methylglycine | |
| 188 | 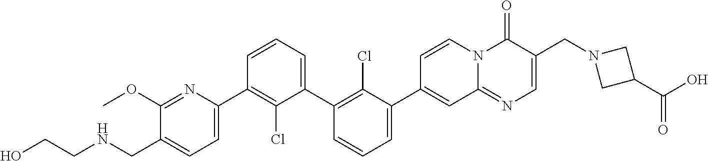 [SMILES: COc1nc(-c2cccc(-c3cccc(-c4ccn5c(=O)c(CN6CC(C(=O)O)C6)cnc5c4)c3Cl)c2Cl)ccc1CNCCO] | 0.0001 |
| | 1-((8-(2,2′-dichloro-3′-(5-(((2-hydroxyethyl)amino)methyl)-6-methoxypyridin-2-yl)-[1,1′-biphenyl]-3-yl)-4-oxo-4H- | |
| | pyrido[1,2-a]pyrimidin-3-yl)methyl)azetidine-3-carboxylic acid | |
| 189 | 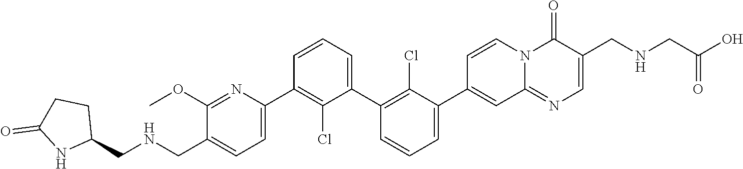 [SMILES: COc1nc(-c2cccc(-c3cccc(-c4ccn5c(=O)c(CNCC(=O)O)cnc5c4)c3Cl)c2Cl)ccc1CNC[C@@H]1CCC(=O)N1] | 0.0001 |
| | (S)-((8-(2,2′-dichloro-3′-(6-methoxy-5-((((5-oxopyrrolidin-2-yl)methyl)amino)methyl)pyridin-2-yl)-[1,1′-biphenyl]-3-yl)-4- | |
| | oxo-4H-pyrido[1,2-a]pyrimidin-3-yl)methyl)glycine | |
| 190 | 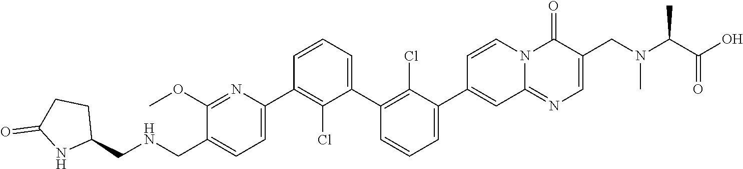 [SMILES: COc1nc(-c2cccc(-c3cccc(-c4ccn5c(=O)c(CN(C)[C@@H](C)C(=O)O)cnc5c4)c3Cl)c2Cl)ccc1CNC[C@@H]1CCC(=O)N1] | 0.0001 |
| | N-((8-(2,2′-dichloro-3′-(6-methoxy-5-(((((S)-5-oxopyrrolidin-2-yl)methyl)amino)methyl)pyridin-2-yl)-[1,1′-biphenyl]-3-yl)-4- | |
| | oxo-4H-pyrido[1,2-a]pyrimidin-3-yl)methyl)-N-methyl-L-alanine | |
| 191 | 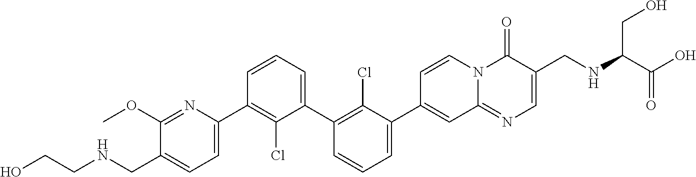 [SMILES: COc1nc(-c2cccc(-c3cccc(-c4ccn5c(=O)c(CN[C@@H](CO)C(=O)O)cnc5c4)c3Cl)c2Cl)ccc1CNCCO] | 0.0001 |
| | ((8-(2,2′-dichloro-3′-(5-(((2-hydroxyethyl)amino)methyl)-6-methoxypyridin-2-yl)-[1,1′-biphenyl]-3-yl)-4-oxo-4H- | |
| | pyrido[1,2-a]pyrimidin-3-yl)methyl)-L-serine | |
| 192 | 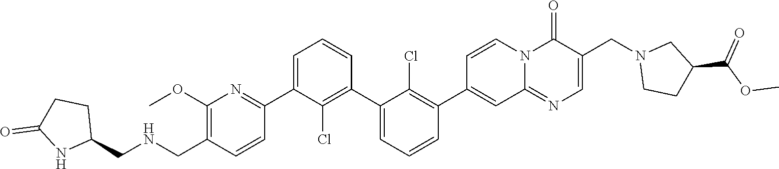 [SMILES: COC(=O)[C@H]1CCN(Cc2cnc3cc(-c4cccc(-c5cccc(-c6ccc(CNC[C@@H]7CCC(=O)N7)c(OC)n6)c5Cl)c4Cl)ccn3c2=O)C1] | 0.0008 |
| | methyl (S)-1-((8-(2,2′-dichloro-3′-(6-methoxy-5-(((((S)-5-oxopyrrolidin-2-yl)methyl)amino)methyl)pyridin-2-yl)-[1,1′-biphenyl]-3- | |
| | yl)-4-oxo-4H-pyrido[1,2-a]pyrimidin-3-yl)methyl)pyrrolidine-3-carboxylate | |
| 193 | 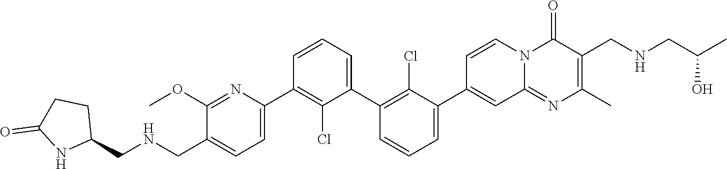 [SMILES: COc1nc(-c2cccc(-c3cccc(-c4ccn5c(=O)c(CNC[C@H](C)O)c(C)nc5c4)c3Cl)c2Cl)ccc1CNC[C@@H]1CCC(=O)N1] | 0.0001 |
| | 8-(2,2′-dichloro-3′-(6-methoxy-5-(((((S)-5-oxopyrrolidin-2-yl)methyl)amino)methyl)pyridin-2-yl)-[1,1′-biphenyl]-3-yl)-3- | |
| | ((((S)-2-hydroxypropyl)amino)methyl)-2-methyl-4H-pyrido[1,2-a]pyrimidin-4-one | |
| 194 | 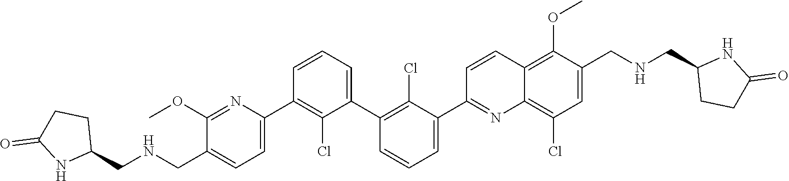 [SMILES: COc1nc(-c2cccc(-c3cccc(-c4ccc5c(OC)c(CNC[C@@H]6CCC(=O)N6)cc(Cl)c5n4)c3Cl)c2Cl)ccc1CNC[C@@H]1CCC(=O)N1] | 0.026 |
| | (S)-5-((((8-chloro-2-(2,2′-dichloro-3′-(6-methoxy-5-(((((S)-5-oxopyrrolidin-2-yl)methyl)amino)methyl)pyridin-2-yl)-[1,1′-biphenyl]-3- | |
| | yl)-5-methoxyquinolin-6-yl)methyl)amino)methyl)pyrrolidin-2-one | |
| 195 | 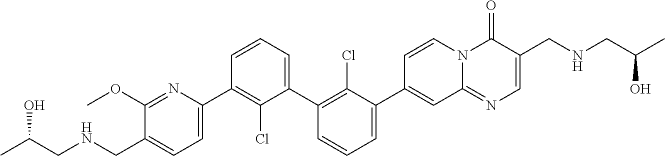 [SMILES: COc1nc(-c2cccc(-c3cccc(-c4ccn5c(=O)c(CNC[C@@H](C)O)cnc5c4)c3Cl)c2Cl)ccc1CNC[C@H](C)O] | 0.0025 |
| | 8-(2,2′-dichloro-3′-(5-((((S)-2-hydroxypropyl)amino)methyl)-6-methoxypyridin-2-yl)-[1,1′-biphenyl]-3-yl)-3- | |
| | ((((R)-2-hydroxypropyl)amino)methyl)-4H-pyrido[1,2-a]pyrimidin-4-one | |
| 196 | 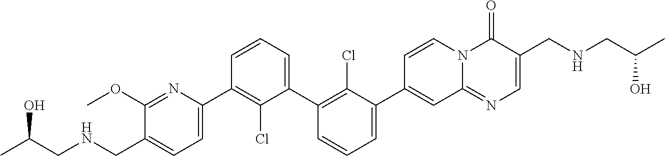 [SMILES: COc1nc(-c2cccc(-c3cccc(-c4ccn5c(=O)c(CNC[C@H](C)O)cnc5c4)c3Cl)c2Cl)ccc1CNC[C@@H](C)O] | 0.0028 |
| | 8-(2,2′-dichloro-3′-(5-((((R)-2-hydroxypropyl)amino)methyl)-6-methoxypyridin-2-yl)-[1,1′-biphenyl]-3-yl)-3- | |
| | ((((S)-2-hydroxypropyl)amino)methyl)-4H-pyrido[1,2-a]pyrimidin-4-one | |
| 197 | 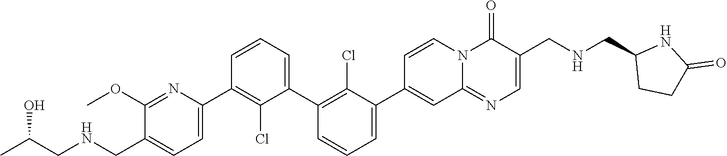 [SMILES: COc1nc(-c2cccc(-c3cccc(-c4ccn5c(=O)c(CNC[C@@H]6CCC(=O)N6)cnc5c4)c3Cl)c2Cl)ccc1CNC[C@H](C)O] | 0.0002 |
| | 8-(2,2′-dichloro-3′-(5-((((S)-2-hydroxypropyl)amino)methyl)-6-methoxypyridin-2-yl)-[1,1′-biphenyl]-3-yl)-3-(((((S)-5- | |
| | oxopyrrolidin-2-yl)methyl)amino)methyl)-4H-pyrido[1,2-a]pyrimidin-4-one | |
| 198 | 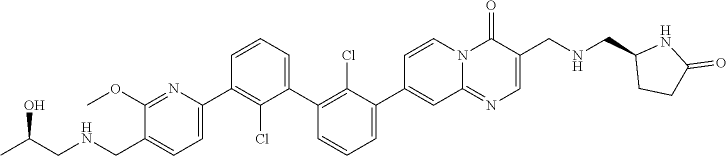 [SMILES: COc1nc(-c2cccc(-c3cccc(-c4ccn5c(=O)c(CNC[C@@H]6CCC(=O)N6)cnc5c4)c3Cl)c2Cl)ccc1CNC[C@@H](C)O] | 0.0003 |
| | 8-(2,2′-dichloro-3′-(5-((((R)-2-hydroxypropyl)amino)methyl)-6-methoxypyridin-2-yl)-[1,1′-biphenyl]-3-yl)-3-(((((S)-5- | |
| | oxopyrrolidin-2-yl)methyl)amino)methyl)-4H-pyrido[1,2-a]pyrimidin-4-one | |
| 199 | 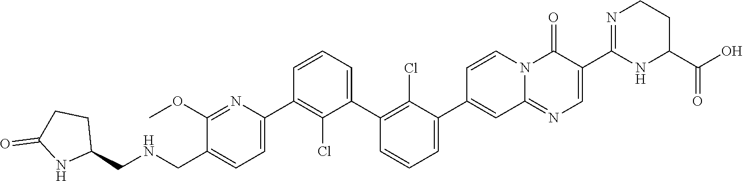 [SMILES: COc1nc(-c2cccc(-c3cccc(-c4ccn5c(=O)c(C6=NCCC(C(=O)O)N6)cnc5c4)c3Cl)c2Cl)ccc1CNC[C@@H]1CCC(=O)N1] | 0.0001 |
| | 2-(8-(2,2′-dichloro-3′-(6-methoxy-5-(((((S)-5-oxopyrrolidin-2-yl)methyl)amino)methyl)pyridin-2-yl)-[1,1′-biphenyl]-3-yl)-4- | |
| | oxo-4H-pyrido[1,2-a]pyrimidin-3-yl)-3,4,5,6-tetrahydropyrimidine-4-carboxylic acid | |
| | mixture of 2 diastereoisomers | |
| 200 | 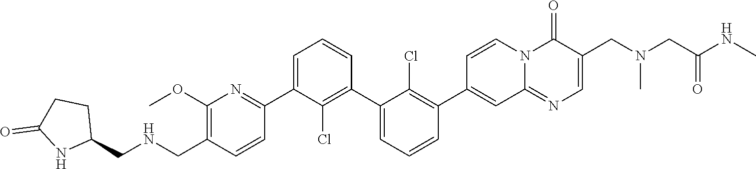 [SMILES: CNC(=O)CN(C)Cc1cnc2cc(-c3cccc(-c4cccc(-c5ccc(CNC[C@@H]6CCC(=O)N6)c(OC)n5)c4Cl)c3Cl)ccn2c1=O] | 0.0001 |
| | (S)-2-(((8-(2,2′-dichloro-3′-(6-methoxy-5-((((5-oxopyrrolidin-2-yl)methyl)amino)methyl)pyridin-2-yl)-[1,1′-biphenyl]-3-yl)-4- | |
| | oxo-4H-pyrido[1,2-a]pyrimidin-3-yl)methyl)(methyl)amino)-N-methylacetamide | |
| 201 | 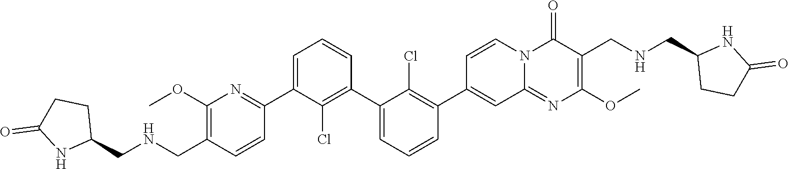 [SMILES: COc1nc(-c2cccc(-c3cccc(-c4ccn5c(=O)c(CNC[C@@H]6CCC(=O)N6)c(OC)nc5c4)c3Cl)c2Cl)ccc1CNC[C@@H]1CCC(=O)N1] | 0.10 |
| | 8-(2,2′-dichloro-3′-(6-methoxy-5-(((((S)-5-oxopyrrolidin-2-yl)methyl)amino)methyl)pyridin-2-yl)-[1,1′-biphenyl]-3-yl)-2-methoxy-3- | |
| | (((((S)-5-oxopyrrolidin-2-yl)methyl)amino)methyl)-4H-pyrido[1,2-a]pyrimidin-4-one | |
| 202 | 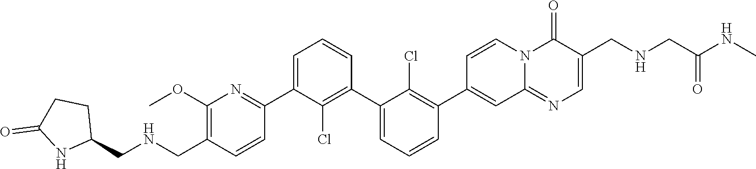 [SMILES: CNC(=O)CNCc1cnc2cc(-c3cccc(-c4cccc(-c5ccc(CNC[C@@H]6CCC(=O)N6)c(OC)n5)c4Cl)c3Cl)ccn2c1=O] | 0.0001 |
| | (S)-2-(((8-(2,2′-dichloro-3′-(6-methoxy-5-((((5-oxopyrrolidin-2-yl)methyl)amino)methyl)pyridin-2-yl)-[1,1′-biphenyl]-3-yl)-4- | |
| | oxo-4H-pyrido[1,2-a]pyrimidin-3-yl)methyl)amino)-N-methylacetamide | |
| 203 | 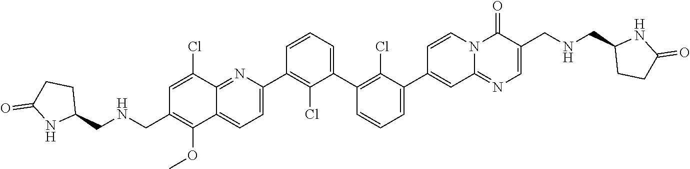 [SMILES: COc1c(CNC[C@@H]2CCC(=O)N2)cc(Cl)c2nc(-c3cccc(-c4cccc(-c5ccn6c(=O)c(CNC[C@@H]7CCC(=O)N7)cnc6c5)c4Cl)c3Cl)ccc12] | 0.15 |
| | 8-(2,2′-dichloro-3′-(8-chloro-5-methoxy-6-(((((S)-5-oxopyrrolidin-2-yl)methyl)amino)methyl)quinolin-2-yl)-[1,1′- | |
| | biphenyl]-3-yl)-3-(((((S)-5-oxopyrrolidin-2-yl)methyl)amino)methyl)-4H-pyrido[1,2-a]pyrimidin-4-one | |
| 204 | 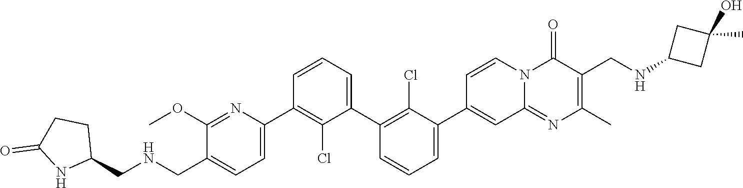 [SMILES: COc1nc(-c2cccc(-c3cccc(-c4ccn5c(=O)c(CN[C@H]6C[C@](C)(O)C6)c(C)nc5c4)c3Cl)c2Cl)ccc1CNC[C@@H]1CCC(=O)N1] | 0.0001 |
| | 8-(2,2′-dichloro-3′-(6-methoxy-5-(((((S)-5-oxopyrrolidin-2-yl)methyl)amino)methyl)pyridin-2-yl)-[1,1′-biphenyl]-3-yl)-3- | |
| | ((((1r,3r)-3-hydroxy-3-methylcyclobutyl)amino)methyl)-2-methyl-4H-pyrido[1,2-a]pyrimidin-4-one | |
| 205 | 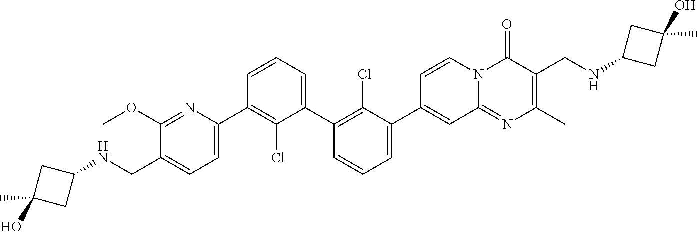 | 0.0009 |
| | 8-(2,2′-dichloro-3′-(5-((((1r,3r)-3-hydroxy-3-methylcyclobutyl)amino)methyl)-6-methoxypyridin-2-yl)-[1,1′-biphenyl]- | |
| | 3-yl)-3-((((1r,3r)-3-hydroxy-3-methylcyclobutyl)amino)methyl)-2-methyl-4H-pyrido[1,2-a]pyrimidin-4-one | |
| 206 | 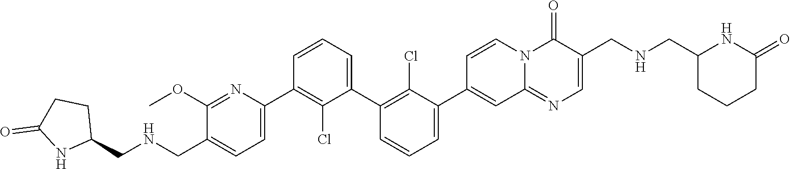 [SMILES: COc1nc(-c2cccc(-c3cccc(-c4ccn5c(=O)c(CNCC6CCCC(=O)N6)cnc5c4)c3Cl)c2Cl)ccc1CNC[C@@H]1CCC(=O)N1] | 0.014 |
| | 8-(2,2′-dichloro-3′-(6-methoxy-5-(((((S)-5-oxopyrrolidin-2-yl)methyl)amino)methyl)pyridin-2-yl)-[1,1′-biphenyl]-3-yl)-3-((((6- | |
| | oxopiperidin-2-yl)methyl)amino)methyl)-4H-pyrido[1,2-a]pyrimidin-4-one | |
| | mixture of 2 diastereoisomers | |
| 207 | 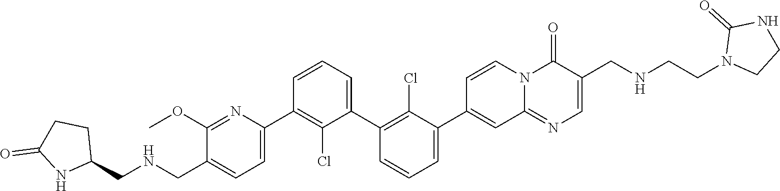 [SMILES: COc1nc(-c2cccc(-c3cccc(-c4ccn5c(=O)c(CNCCN6CCNC6=O)cnc5c4)c3Cl)c2Cl)ccc1CNC[C@@H]1CCC(=O)N1] | 0.0003 |
| | (S)-8-(2,2′-dichloro-3′-(6-methoxy-5-((((5-oxopyrrolidin-2-yl)methyl)amino)methyl)pyridin-2-yl)-[1,1′-biphenyl]-3-yl)-3-(((2-(2- | |
| | oxoimidazolidin-1-yl)ethyl)amino)methyl)-4H-pyrido[1,2-a]pyrimidin-4-one | |
| 208 | 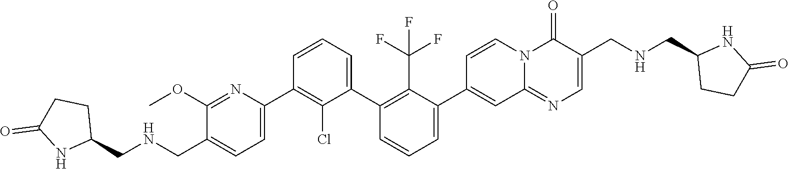 [SMILES: COc1nc(-c2cccc(-c3cccc(-c4ccn5c(=O)c(CNC[C@@H]6CCC(=O)N6)cnc5c4)c3C(F)(F)F)c2Cl)ccc1CNC[C@@H]1CCC(=O)N1] | 0.038 |
| | 8-(2′-chloro-3′-(6-methoxy-5-(((((S)-5-oxopyrrolidin-2-yl)methyl)amino)methyl)pyridin-2-yl)-2-(trifluoromethyl)-[1,1′-biphenyl]-3- | |
| | yl)-3-(((((S)-5-oxopyrrolidin-2-yl)methyl)amino)methyl)-4H-pyrido[1,2-a]pyrimidin-4-one | |
| 209 | 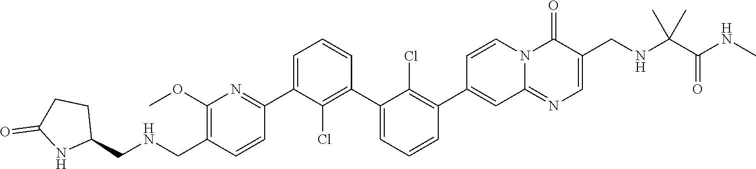 [SMILES: CNC(=O)C(C)(C)NCc1cnc2cc(-c3cccc(-c4cccc(-c5ccc(CNC[C@@H]6CCC(=O)N6)c(OC)n5)c4Cl)c3Cl)ccn2c1=O] | 0.0001 |
| | (S)-2-(((8-(2,2′-dichloro-3′-(6-methoxy-5-((((5-oxopyrrolidin-2-yl)methyl)amino)methyl)pyridin-2-yl)-[1,1′-biphenyl]-3-yl)-4- | |
| | oxo-4H-pyrido[1,2-a]pyrimidin-3-yl)methyl)amino)-N,2-dimethylpropanamide | |
| 210 | 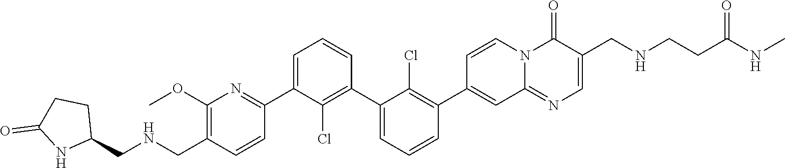 [SMILES: CNC(=O)CCNCc1cnc2cc(-c3cccc(-c4cccc(-c5ccc(CNC[C@@H]6CCC(=O)N6)c(OC)n5)c4Cl)c3Cl)ccn2c1=O] | 0.0001 |
| | (S)-3-(((8-(2,2′-dichloro-3′-(6-methoxy-5-((((5-oxopyrrolidin-2-yl)methyl)amino)methyl)pyridin-2-yl)-[1,1′-biphenyl]-3-yl)-4-oxo- | |
| | 4H-pyrido[1,2-a]pyrimidin-3-yl)methyl)amino)-N-methylpropanamide | |
| 211 | 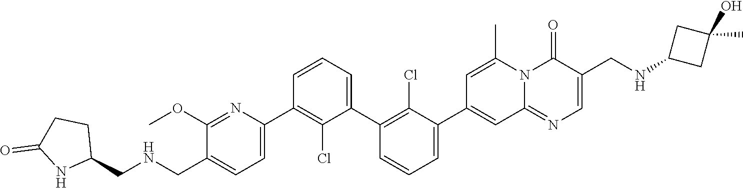 [SMILES: COc1nc(-c2cccc(-c3cccc(-c4cc(C)n5c(=O)c(CN[C@H]6C[C@](C)(O)C6)cnc5c4)c3Cl)c2Cl)ccc1CNC[C@@H]1CCC(=O)N1] | 0.021 |
| | 8-(2,2′-dichloro-3′-(6-methoxy-5-(((((S)-5-oxopyrrolidin-2-yl)methyl)amino)methyl)pyridin-2-yl)-[1,1′-biphenyl]-3-yl)-3- | |
| | ((((1r,3r)-3-hydroxy-3-methylcyclobutyl)amino)methyl)-6-methyl-4H-pyrido[1,2-a]pyrimidin-4-one | |
| 212 | 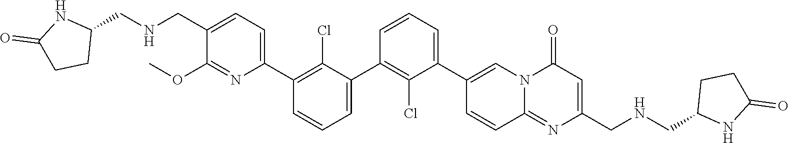 [SMILES: COc1nc(-c2cccc(-c3cccc(-c4ccc5nc(CNC[C@@H]6CCC(=O)N6)cc(=O)n5c4)c3Cl)c2Cl)ccc1CNC[C@@H]1CCC(=O)N1] | 0.0001 |
| | 7-(2,2′-dichloro-3′-(6-methoxy-5-(((((S)-5-oxopyrrolidin-2-yl)methyl)amino)methyl)pyridin-2-yl)-[1,1′-biphenyl]-3-yl)-2-(((((S)-5- | |
| | oxopyrrolidin-2-yl)methyl)amino)methyl)-4H-pyrido[1,2-a]pyrimidin-4-one | |
| 213 | 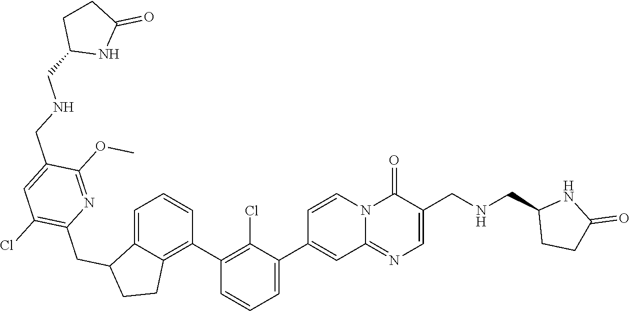 [SMILES: COc1nc(CC2CCc3c(-c4cccc(-c5ccn6c(=O)c(CNC[C@@H]7CCC(=O)N7)cnc6c5)c4Cl)cccc32)c(Cl)cc1CNC[C@@H]1CCC(=O)N1] | 3.7 |
| | 8-(2-chloro-3-(1-((3-chloro-6-methoxy-5-(((((S)-5-oxopyrrolidin-2-yl)methyl)amino)methyl)pyridin-2- | |
| | yl)methyl)-2,3-dihydro-1H-inden-4-yl)phenyl)-3-(((((S)-5-oxopyrrolidin-2-yl)methyl)amino)methyl)-4H- | |
| | pyrido[1,2-a]pyrimidin-4-one | |
| | mixture of 2 diastereoisomers | |
| 214 | 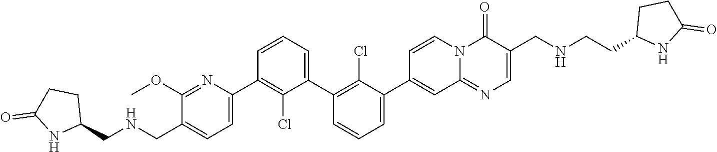 [SMILES: COc1nc(-c2cccc(-c3cccc(-c4ccn5c(=O)c(CNCC[C@@H]6CCC(=O)N6)cnc5c4)c3Cl)c2Cl)ccc1CNC[C@@H]1CCC(=O)N1] | 0.0005 |
| | 8-(2,2′-dichloro-3′-(6-methoxy-5-(((((S)-5-oxopyrrolidin-2-yl)methyl)amino)methyl)pyridin-2-yl)-[1,1′-biphenyl]-3-yl)-3- | |
| | (((2-((S)-5-oxopyrrolidin-2-yl)ethyl)amino)methyl)-4H-pyrido[1,2-a]pyrimidin-4-one | |
| 215 | 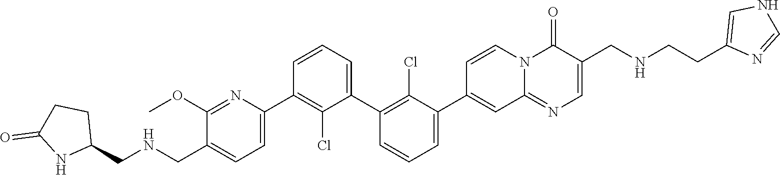 [SMILES: COc1nc(-c2cccc(-c3cccc(-c4ccn5c(=O)c(CNCCc6c[nH]cn6)cnc5c4)c3Cl)c2Cl)ccc1CNC[C@@H]1CCC(=O)N1] | 0.0080 |
| | (S)-3-(((2-(1H-imidazol-4-yl)ethyl)amino)methyl)-8-(2,2′-dichloro-3′-(6-methoxy-5-((((5-oxopyrrolidin-2- | |
| | yl)methyl)amino)methyl)pyridin-2-yl)-[1,1′-biphenyl]-3-yl)-4H-pyrido[1,2-a]pyrimidin-4-one | |
| 216 | 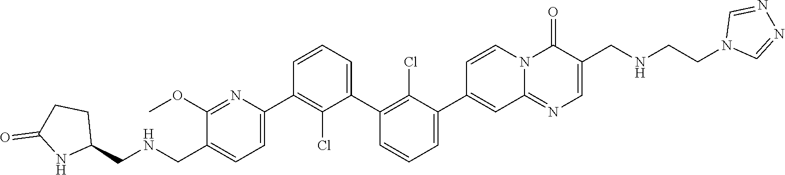 [SMILES: COc1nc(-c2cccc(-c3cccc(-c4ccn5c(=O)c(CNCCn6cnnc6)cnc5c4)c3Cl)c2Cl)ccc1CNC[C@@H]1CCC(=O)N1] | 0.0001 |
| | (S)-3-(((2-(4H-1,2,4-triazol-4-yl)ethyl)amino)methyl)-8-(2,2′-dichloro-3′-(6-methoxy-5-((((5-oxopyrrolidin-2- | |
| | yl)methyl)amino)methyl)pyridin-2-yl)-[1,1′-biphenyl]-3-yl)-4H-pyrido[1,2-a]pyrimidin-4-one | |
| 217 | 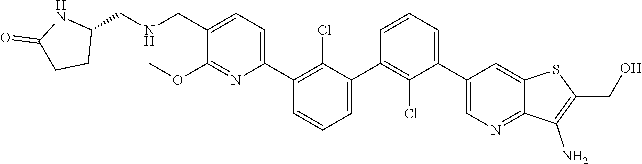 [SMILES: COc1nc(-c2cccc(-c3cccc(-c4cnc5c(N)c(CO)sc5c4)c3Cl)c2Cl)ccc1CNC[C@@H]1CCC(=O)N1] | 0.0035 |
| | (S)-5-((((6-(3′-(3-amino-2-(hydroxymethyl)thieno[3,2-b]pyridin-6-yl)-2,2′-dichloro-[1,1′-biphenyl]-3-yl)-2- | |
| | methoxypyridin-3-yl)methyl)amino)methyl)pyrrolidin-2-one | |
| 218 | 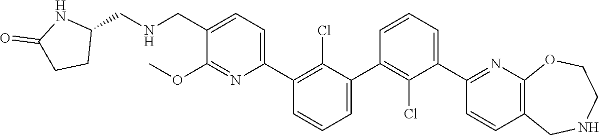 [SMILES: COc1nc(-c2cccc(-c3cccc(-c4ccc5c(n4)OCCNC5)c3Cl)c2Cl)ccc1CNC[C@@H]1CCC(=O)N1] | 0.0006 |
| | (S)-5-((((6-(2,2′-dichloro-3′-(2,3,4,5-tetrahydropyrido[3,2-f][1,4]oxazepin-8-yl)-[1,1′-biphenyl]-3-yl)- | |
| | 2-methoxypyridin-3-yl)methyl)amino)methyl)pyrrolidin-2-one | |
| 219 | 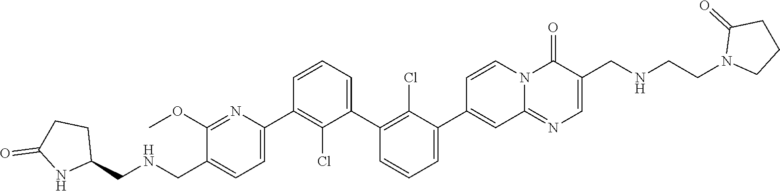 [SMILES: COc1nc(-c2cccc(-c3cccc(-c4ccn5c(=O)c(CNCCN6CCCC6=O)cnc5c4)c3Cl)c2Cl)ccc1CNC[C@@H]1CCC(=O)N1] | 0.0007 |
| | (S)-8-(2,2′-dichloro-3′-(6-methoxy-5-((((5-oxopyrrolidin-2-yl)methyl)amino)methyl)pyridin-2-yl)-[1,1′-biphenyl]-3-yl)-3-(((2-(2- | |
| | oxopyrrolidin-1-yl)ethyl)amino)methyl)-4H-pyrido[1,2-a]pyrimidin-4-one | |
| 220 | 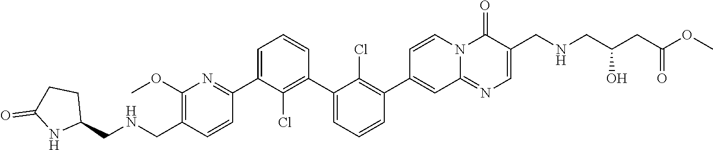 [SMILES: COC(=O)C[C@H](O)CNCc1cnc2cc(-c3cccc(-c4cccc(-c5ccc(CNC[C@@H]6CCC(=O)N6)c(OC)n5)c4Cl)c3Cl)ccn2c1=O] | 0.0001 |
| | methyl (S)-4-(((8-(2,2′-dichloro-3′-(6-methoxy-5-(((((S)-5-oxopyrrolidin-2-yl)methyl)amino)methyl)pyridin-2-yl)-[1,1′- | |
| | biphenyl]-3-yl)-4-oxo-4H-pyrido[1,2-a]pyrimidin-3-yl)methyl)amino)-3-hydroxybutanoate | |
| 221 | 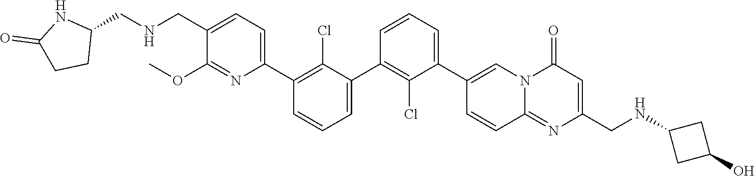 [SMILES: COc1nc(-c2cccc(-c3cccc(-c4ccc5nc(CN[C@H]6C[C@H](O)C6)cc(=O)n5c4)c3Cl)c2Cl)ccc1CNC[C@@H]1CCC(=O)N1] | 0.0006 |
| | 7-(2,2′-dichloro-3′-(6-methoxy-5-(((((S)-5-oxopyrrolidin-2-yl)methyl)amino)methyl)pyridin-2-yl)-[1,1′-biphenyl]-3-yl)-2- | |
| | ((((1r,3r)-3-hydroxycyclobutyl)amino)methyl)-4H-pyrido[1,2-a]pyrimidin-4-one | |
| 222 | 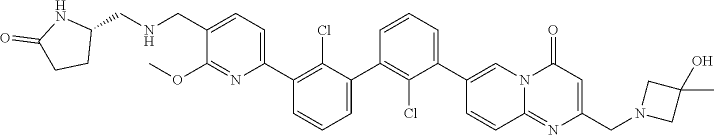 [SMILES: COc1nc(-c2cccc(-c3cccc(-c4ccc5nc(CN6CC(C)(O)C6)cc(=O)n5c4)c3Cl)c2Cl)ccc1CNC[C@@H]1CCC(=O)N1] | 0.0002 |
| | (S)-7-(2,2′-dichloro-3′-(6-methoxy-5-((((5-oxopyrrolidin-2-yl)methyl)amino)methyl)pyridin-2-yl)-[1,1′-biphenyl]-3-yl)- | |
| | 2-((3-hydroxy-3-methylazetidin-1-yl)methyl)-4H-pyrido[1,2-a]pyrimidin-4-one | |
| 223 | 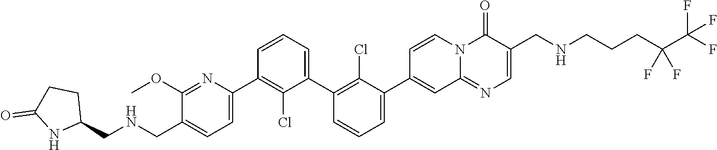 [SMILES: COc1nc(-c2cccc(-c3cccc(-c4ccn5c(=O)c(CNCCCC(F)(F)C(F)(F)F)cnc5c4)c3Cl)c2Cl)ccc1CNC[C@@H]1CCC(=O)N1] | 0.010 |
| | (S)-8-(2,2′-dichloro-3′-(6-methoxy-5-((((5-oxopyrrolidin-2-yl)methyl)amino)methyl)pyridin-2-yl)-[1,1′-biphenyl]-3-yl)- | |
| | 3-(((4,4,5,5,5-pentafluoropentyl)amino)methyl)-4H-pyrido[1,2-a]pyrimidin-4-one | |
| 224 | 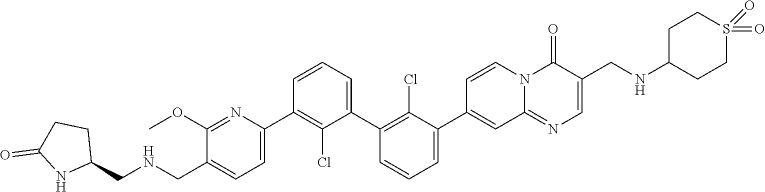 [SMILES: COc1nc(-c2cccc(-c3cccc(-c4ccn5c(=O)c(CNC6CCS(=O)(=O)CC6)cnc5c4)c3Cl)c2Cl)ccc1CNC[C@@H]1CCC(=O)N1] | 0.0028 |
| | (S)-8-(2,2′-dichloro-3′-(6-methoxy-5-((((5-oxopyrrolidin-2-yl)methyl)amino)methyl)pyridin-2-yl)-[1,1′-biphenyl]-3-yl)-3-(((1,1- | |
| | dioxidotetrahydro-2H-thiopyran-4-yl)amino)methyl)-4H-pyrido[1,2-a]pyrimidin-4-one | |
| 225 | 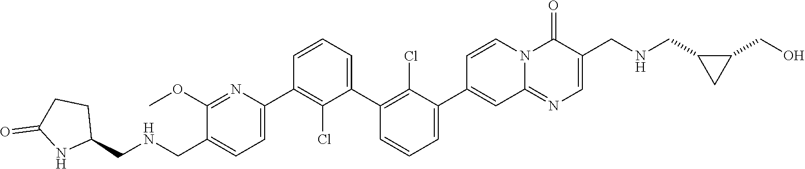 | 0.0002 |
| | cis-8-(2,2′-dichloro-3′-(6-methoxy-5-(((((S)-5-oxopyrrolidin-2-yl)methyl)amino)methyl)pyridin-2-yl)-[1,1′-biphenyl]-3-yl)-3-((((2- | |
| | (hydroxymethyl)cyclopropyl)methyl)amino)methyl)-4H-pyrido[1,2-a]pyrimidin-4-one | |
| | mixture of 2 diastereoisomers | |
| 226 | 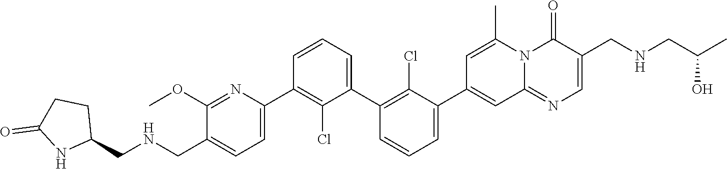 [SMILES: COc1nc(-c2cccc(-c3cccc(-c4cc(C)n5c(=O)c(CNC[C@H](C)O)cnc5c4)c3Cl)c2Cl)ccc1CNC[C@@H]1CCC(=O)N1] | 0.015 |
| | 8-(2,2′-dichloro-3′-(6-methoxy-5-(((((S)-5-oxopyrrolidin-2-yl)methyl)amino)methyl)pyridin-2-yl)-[1,1′-biphenyl]-3-yl)-3- | |
| | ((((S)-2-hydroxypropyl)amino)methyl)-6-methyl-4H-pyrido[1,2-a]pyrimidin-4-one | |
| 227 | 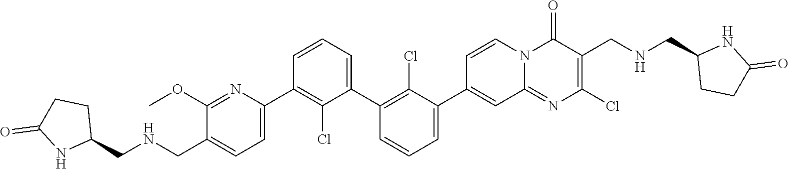 [SMILES: COc1nc(-c2cccc(-c3cccc(-c4ccn5c(=O)c(CNC[C@@H]6CCC(=O)N6)c(Cl)nc5c4)c3Cl)c2Cl)ccc1CNC[C@@H]1CCC(=O)N1] | 0.012 |
| | 2-chloro-8-(2,2′-dichloro-3′-(6-methoxy-5-(((((S)-5-oxopyrrolidin-2-yl)methyl)amino)methyl)pyridin-2-yl)-[1,1′-biphenyl]-3-yl)-3- | |
| | (((((S)-5-oxopyrrolidin-2-yl)methyl)amino)methyl)-4H-pyrido[1,2-a]pyrimidin-4-one | |
| 228 | 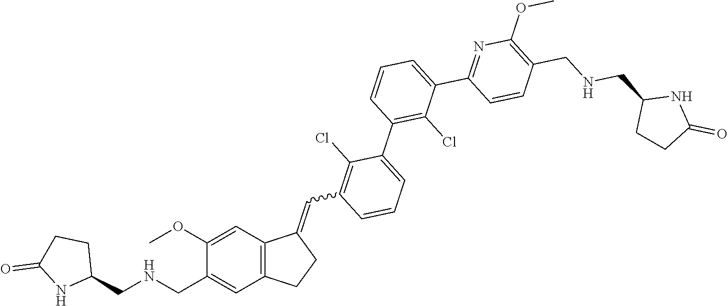 [SMILES: COc1cc2c(cc1CNC[C@@H]1CCC(=O)N1)CCC2=Cc1cccc(-c2cccc(-c3ccc(CNC[C@@H]4CCC(=O)N4)c(OC)n3)c2Cl)c1Cl] | 3.20 |
| | (S)-5-((((6-(2,2′-dichloro-3′-((6-methoxy-5-(((((S)-5-oxopyrrolidin-2-yl)methyl)amino)methyl)-2,3-dihydro-1H-inden-1- | |
| | ylidene)methyl)-[1,1′-biphenyl]-3-yl)-2-methoxypyridin-3-yl)methyl)amino)methyl)pyrrolidin-2-one | |
| 229 | 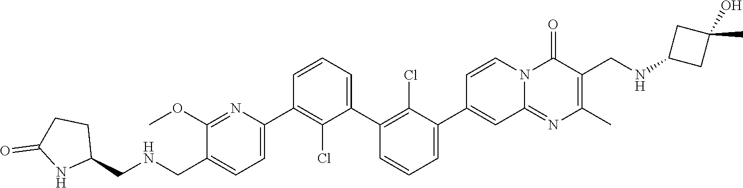 [SMILES: COc1nc(-c2cccc(-c3cccc(-c4ccn5c(=O)c(CN[C@H]6C[C@@](C)(O)C6)c(C)nc5c4)c3Cl)c2Cl)ccc1CNC[C@@H]1CCC(=O)N1] | 0.0069 |
| | 8-(2,2′-dichloro-3′-(6-methoxy-5-(((((S)-5-oxopyrrolidin-2-yl)methyl)amino)methyl)pyridin-2-yl)-[1,1′-biphenyl]-3-yl)-3- | |
| | ((((1s,3s)-3-hydroxy-3-methylcyclobutyl)amino)methyl)-2-methyl-4H-pyrido[1,2-a]pyrimidin-4-one | |
| 230 | 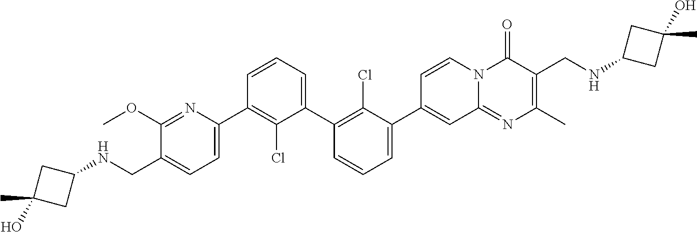 | 0.098 |
| | 8-(2,2′-dichloro-3′-(5-((((1s,3s)-3-hydroxy-3-methylcyclobutyl)amino)methyl)-6-methoxypyridin-2-yl)-[1,1′-biphenyl]- | |
| | 3-yl)-3-((((1s,3s)-3-hydroxy-3-methylcyclobutyl)amino)methyl)-2-methyl-4H-pyrido[1,2-a]pyrimidin-4-one | |
| 231 | 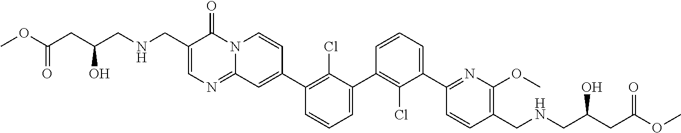 | 0.12 |
| | methyl (S)-4-(((6-(2,2′-dichloro-3′-(3-((((S)-2-hydroxy-4-methoxy-4-oxobutyl)amino)methyl)-4-oxo-4H- | |
| | pyrido[1,2-a]pyrimidin-8-yl)-[1,1′-biphenyl]-3-yl)-2-methoxypyridin-3-yl)methyl)amino)-3-hydroxybutanoate | |
| 232 | 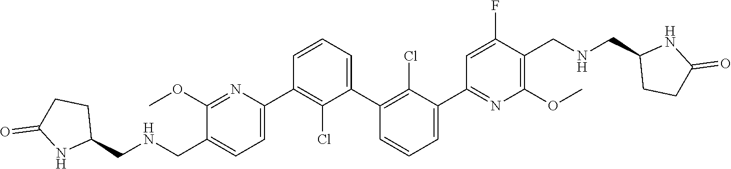 [SMILES: COc1nc(-c2cccc(-c3cccc(-c4cc(F)c(CNC[C@@H]5CCC(=O)N5)c(OC)n4)c3Cl)c2Cl)ccc1CNC[C@@H]1CCC(=O)N1] | 0.063 |
| | (S)-5-((((6-(2,2′-dichloro-3′-(4-fluoro-6-methoxy-5-(((((S)-5-oxopyrrolidin-2-yl)methyl)amino)methyl)pyridin-2-yl)-[1,1′- | |
| | biphenyl]-3-yl)-2-methoxypyridin-3-yl)methyl)amino)methyl)pyrrolidin-2-one | |
| 233 | 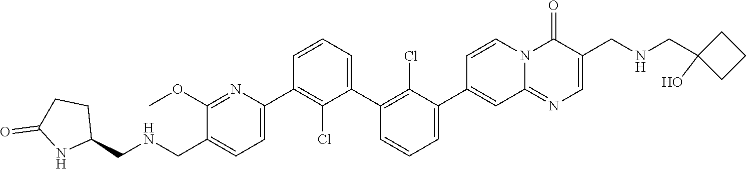 [SMILES: COc1nc(-c2cccc(-c3cccc(-c4ccn5c(=O)c(CNCC6(O)CCC6)cnc5c4)c3Cl)c2Cl)ccc1CNC[C@@H]1CCC(=O)N1] | 0.0002 |
| | (S)-8-(2,2′-dichloro-3′-(6-methoxy-5-((((5-oxopyrrolidin-2-yl)methyl)amino)methyl)pyridin-2-yl)-[1,1′-biphenyl]-3-yl)-3-((((1- | |
| | hydroxycyclobutyl)methyl)amino)methyl)-4H-pyrido[1,2-a]pyrimidin-4-one | |
| 234 | 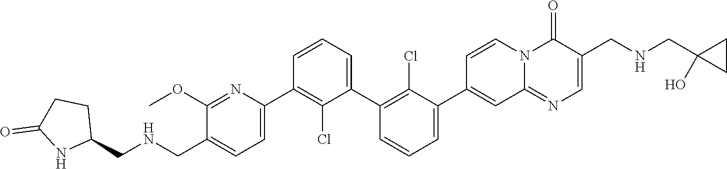 [SMILES: COc1nc(-c2cccc(-c3cccc(-c4ccn5c(=O)c(CNCC6(O)CC6)cnc5c4)c3Cl)c2Cl)ccc1CNC[C@@H]1CCC(=O)N1] | 0.0001 |
| | (S)-8-(2,2′-dichloro-3′-(6-methoxy-5-((((5-oxopyrrolidin-2-yl)methyl)amino)methyl)pyridin-2-yl)-[1,1′-biphenyl]-3-yl)-3- | |
| | ((((1-hydroxycyclopropyl)methyl)amino)methyl)-4H-pyrido[1,2-a]pyrimidin-4-one | |
| 235 | 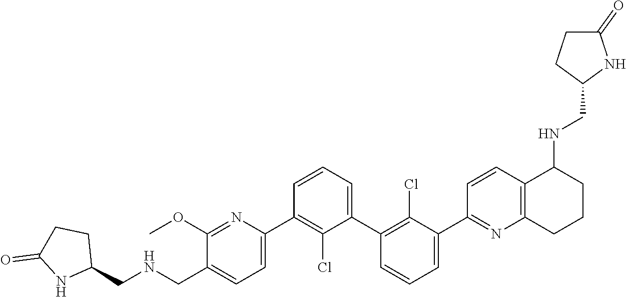 [SMILES: COc1nc(-c2cccc(-c3cccc(-c4ccc5c(n4)CCCC5NC[C@@H]4CCC(=O)N4)c3Cl)c2Cl)ccc1CNC[C@@H]1CCC(=O)N1] | 0.0010 |
| | (5S)-5-((((6-(2,2′-dichloro-3′-(5-((((S)-5-oxopyrrolidin-2-yl)methyl)amino)-5,6,7,8-tetrahydroquinolin-2- | |
| | yl)-[1,1′-biphenyl]-3-yl)-2-methoxypyridin-3-yl)methyl)amino)methyl)pyrrolidin-2-one | |
| | mixture of 2 diastereoisomers | |
| 236 | 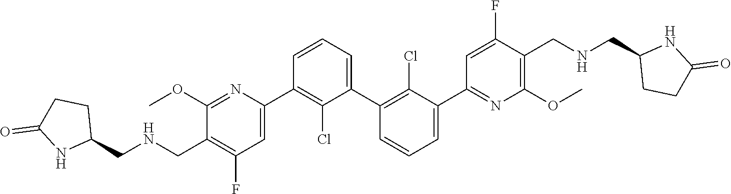 [SMILES: COc1nc(-c2cccc(-c3cccc(-c4cc(F)c(CNC[C@@H]5CCC(=O)N5)c(OC)n4)c3Cl)c2Cl)cc(F)c1CNC[C@@H]1CCC(=O)N1] | 0.0012 |
| | (5S,5′S)-5,5′-(((((2,2′-dichloro-[1,1′-biphenyl]-3,3′-diyl)bis(4-fluoro-2-methoxypyridine-6,3- | |
| | diyl))bis(methylene))bis(azanediyl))bis(methylene))bis(pyrrolidin-2-one) | |
| 237 | 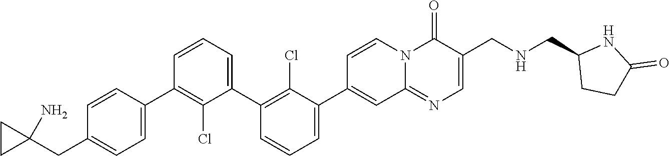 [SMILES: NC1(Cc2ccc(-c3cccc(-c4cccc(-c5ccn6c(=O)c(CNC[C@@H]7CCC(=O)N7)cnc6c5)c4Cl)c3Cl)cc2)CC1] | 0.90 |
| | (S)-8-(4″-((1-aminocyclopropyl)methyl)-2,2′-dichloro-[1,1′:3′,1″-terphenyl]-3-yl)-3-((((5-oxopyrrolidin-2- | |
| | yl)methyl)amino)methyl)-4H-pyrido[1,2-a]pyrimidin-4-one | |
| 238 | 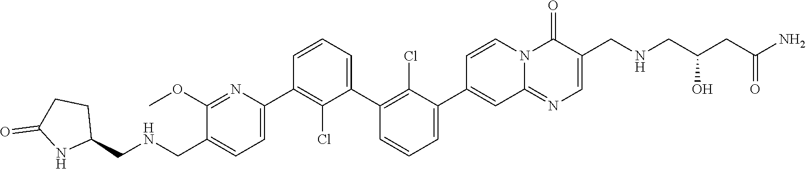 [SMILES: COc1nc(-c2cccc(-c3cccc(-c4ccn5c(=O)c(CNC[C@@H](O)CC(N)=O)cnc5c4)c3Cl)c2Cl)ccc1CNC[C@@H]1CCC(=O)N1] | 0.0001 |
| | (S)-4-(((8-(2,2′-dichloro-3′-(6-methoxy-5-(((((S)-5-oxopyrrolidin-2-yl)methyl)amino)methyl)pyridin-2-yl)-[1,1′-biphenyl]-3-yl)-4-oxo- | |
| | 4H-pyrido[1,2-a]pyrimidin-3-yl)methyl)amino)-3-hydroxybutanamide | |
| 239 | 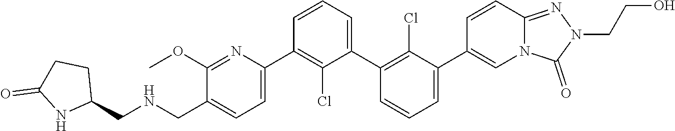 [SMILES: COc1nc(-c2cccc(-c3cccc(-c4ccc5nn(CCO)c(=O)n5c4)c3Cl)c2Cl)ccc1CNC[C@@H]1CCC(=O)N1] | 0.0001 |
| | (S)-6-(2,2′-dichloro-3′-(6-methoxy-5-((((5-oxopyrrolidin-2-yl)methyl)amino)methyl)pyridin-2-yl)-[1,1′-biphenyl]- | |
| | 3-yl)-2-(2-hydroxyethyl)-[1,2,4]triazolo[4,3-a]pyridin-3(2H)-one | |
| 240 | 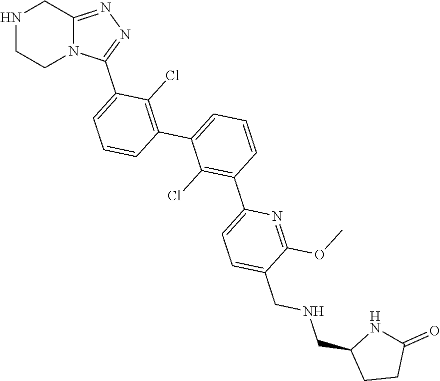 [SMILES: COc1nc(-c2cccc(-c3cccc(-c4nnc5n4CCNC5)c3Cl)c2Cl)ccc1CNC[C@@H]1CCC(=O)N1] | 0.35 |
| | (S)-5-((((6-(2,2′-dichloro-3′-(5,6,7,8-tetrahydro-[1,2,4]triazolo[4,3- | |
| | a]pyrazin-3-yl)-[1,1′-biphenyl]-3-yl)-2-methoxypyridin-3- | |
| | yl)methyl)amino)methyl)pyrrolidin-2-one | |
| 241 | 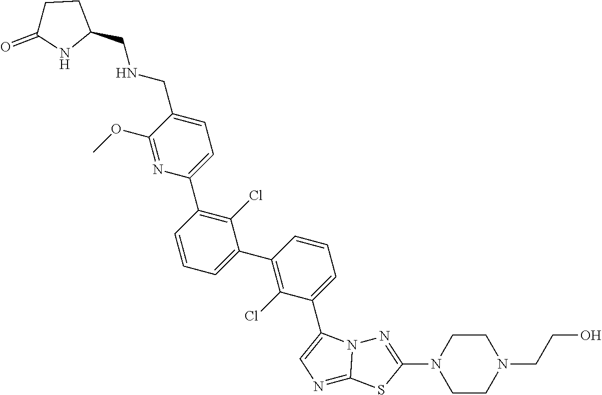 [SMILES: COc1nc(-c2cccc(-c3cccc(-c4cnc5sc(N6CCN(CCO)CC6)nn45)c3Cl)c2Cl)ccc1CNC[C@@H]1CCC(=O)N1] | 0.90 |
| | (S)-5-((((6-(2,2′-dichloro-3′-(2-(4-(2-hydroxyethyl)piperazin-1-yl)imidazo[2,1-b][1,3,4]thiadiazol-5-yl)- | |
| | [1,1′-biphenyl]-3-yl)-2-methoxypyridin-3-yl)methyl)amino)methyl)pyrrolidin-2-one | |
| 242 | 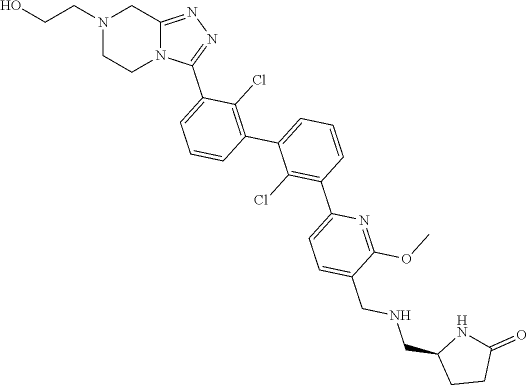 [SMILES: COc1nc(-c2cccc(-c3cccc(-c4nnc5n4CCN(CCO)C5)c3Cl)c2Cl)ccc1CNC[C@@H]1CCC(=O)N1] | 1.7 |
| | (S)-5-((((6-(2,2′-dichloro-3′-(7-(2-hydroxyethyl)-5,6,7,8-tetrahydro-[1,2,4]triazolo[4,3- | |
| | a]pyrazin-3-yl)-[1,1′-biphenyl]-3-yl)-2-methoxypyridin-3- | |
| | yl)methyl)amino)methyl)pyrrolidin-2-one | |
| 243 | 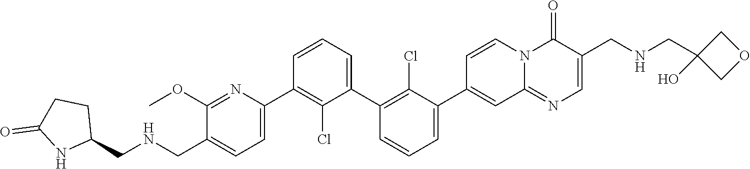 [SMILES: COc1nc(-c2cccc(-c3cccc(-c4ccn5c(=O)c(CNCC6(O)COC6)cnc5c4)c3Cl)c2Cl)ccc1CNC[C@@H]1CCC(=O)N1] | 0.0003 |
| | (S)-8-(2,2′-dichloro-3′-(6-methoxy-5-((((5-oxopyrrolidin-2-yl)methyl)amino)methyl)pyridin-2-yl)-[1,1′-biphenyl]-3-yl)-3-((((3- | |
| | hydroxyoxetan-3-yl)methyl)amino)methyl)-4H-pyrido[1,2-a]pyrimidin-4-one | |
| 244 | 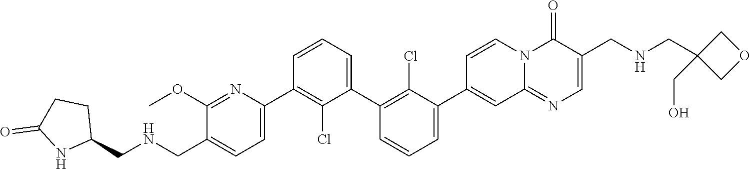 [SMILES: COc1nc(-c2cccc(-c3cccc(-c4ccn5c(=O)c(CNCC6(CO)COC6)cnc5c4)c3Cl)c2Cl)ccc1CNC[C@@H]1CCC(=O)N1] | 0.0006 |
| | (S)-8-(2,2′-dichloro-3′-(6-methoxy-5-((((5-oxopyrrolidin-2-yl)methyl)amino)methyl)pyridin-2-yl)-[1,1′-biphenyl]-3-yl)-3-((((3- | |
| | (hydroxymethyl)oxetan-3-yl)methyl)amino)methyl)-4H-pyrido[1,2-a]pyrimidin-4-one | |
| 245 | 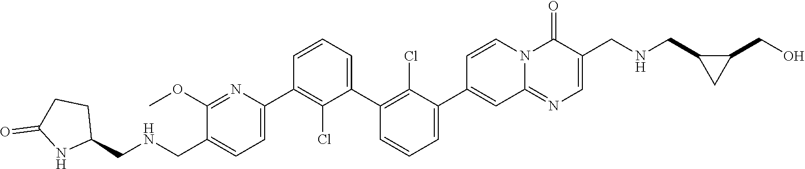 | 0.0021 |
| | cis-8-(2,2′-dichloro-3′-(6-methoxy-5-(((((S)-5-oxopyrrolidin-2-yl)methyl)amino)methyl)pyridin-2-yl)-[1,1′-biphenyl]-3-yl)-3-((((2- | |
| | (hydroxymethyl)cyclopropyl)methyl)amino)methyl)-4H-pyrido[1,2-a]pyrimidin-4-one | |
| | mixture of 2 diastereoisomers | |
| 246 | 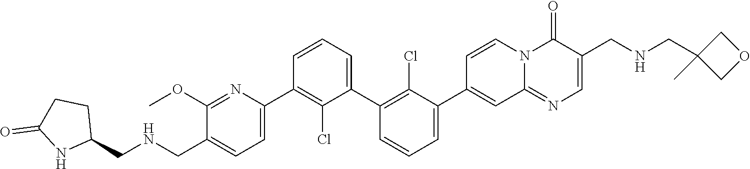 [SMILES: COc1nc(-c2cccc(-c3cccc(-c4ccn5c(=O)c(CNCC6(C)COC6)cnc5c4)c3Cl)c2Cl)ccc1CNC[C@@H]1CCC(=O)N1] | 0.0067 |
| | (S)-8-(2,2′-dichloro-3′-(6-methoxy-5-((((5-oxopyrrolidin-2-yl)methyl)amino)methyl)pyridin-2-yl)-[1,1′-biphenyl]-3-yl)-3-((((3- | |
| | methyloxetan-3-yl)methyl)amino)methyl)-4H-pyrido[1,2-a]pyrimidin-4-one | |
| 247 | 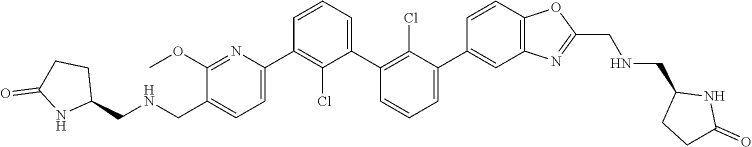 [SMILES: COc1nc(-c2cccc(-c3cccc(-c4ccc5oc(CNC[C@@H]6CCC(=O)N6)nc5c4)c3Cl)c2Cl)ccc1CNC[C@@H]1CCC(=O)N1] | 0.45 |
| | (S)-5-((((6-(2,2′-dichloro-3′-(2-(((((S)-5-oxopyrrolidin-2-yl)methyl)amino)methyl)benzo[d]oxazol-5-yl)-[1,1′-biphenyl]-3-yl)-2- | |
| | methoxypyridin-3-yl)methyl)amino)methyl)pyrrolidin-2-one | |
| 248 | 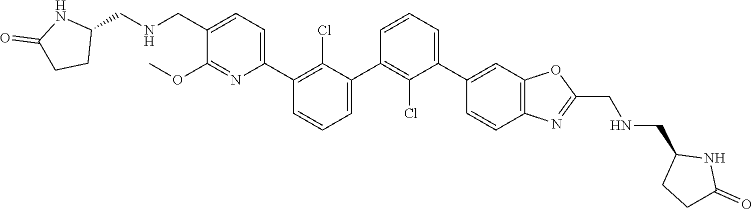 [SMILES: COc1nc(-c2cccc(-c3cccc(-c4ccc5nc(CNC[C@@H]6CCC(=O)N6)oc5c4)c3Cl)c2Cl)ccc1CNC[C@@H]1CCC(=O)N1] | 0.25 |
| | (S)-5-((((6-(2,2′-dichloro-3′-(2-(((((S)-5-oxopyrrolidin-2-yl)methyl)amino)methyl)benzo[d]oxazol-6-yl)-[1,1′-biphenyl]-3-yl)-2- | |
| | methoxypyridin-3-yl)methyl)amino)methyl)pyrrolidin-2-one | |
| 249 | 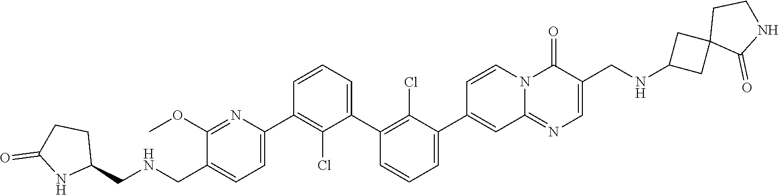 [SMILES: COc1nc(-c2cccc(-c3cccc(-c4ccn5c(=O)c(CNC6CC7(CCNC7=O)C6)cnc5c4)c3Cl)c2Cl)ccc1CNC[C@@H]1CCC(=O)N1] | 0.0006 |
| | (S)-8-(2,2′-dichloro-3′-(6-methoxy-5-((((5-oxopyrrolidin-2-yl)methyl)amino)methyl)pyridin-2-yl)-[1,1′-biphenyl]-3-yl)-3-(((5-oxo-6- | |
| | azaspiro[3.4]octan-2-yl)amino)methyl)-4H-pyrido[1,2-a]pyrimidin-4-one | |
| | mixture of 2 diastereoisomers | |
| 250 | 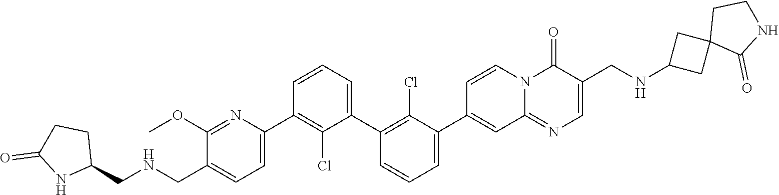 [SMILES: COc1nc(-c2cccc(-c3cccc(-c4ccn5c(=O)c(CNC6CC7(CCNC7=O)C6)cnc5c4)c3Cl)c2Cl)ccc1CNC[C@@H]1CCC(=O)N1] | 0.0010 |
| | (S)-8-(2,2′-dichloro-3′-(6-methoxy-5-((((5-oxopyrrolidin-2-yl)methyl)amino)methyl)pyridin-2-yl)-[1,1′-biphenyl]-3-yl)-3-(((5-oxo- | |
| | 6-azaspiro[3.4]octan-2-yl)amino)methyl)-4H-pyrido[1,2-a]pyrimidin-4-one | |
| | diastereoisomer I | |
| 251 | 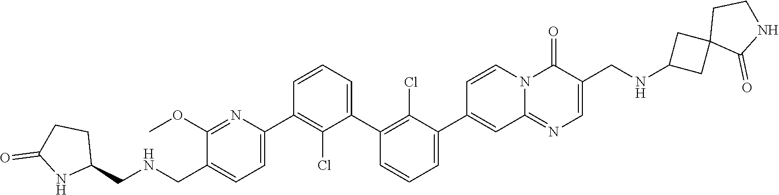 [SMILES: COc1nc(-c2cccc(-c3cccc(-c4ccn5c(=O)c(CNC6CC7(CCNC7=O)C6)cnc5c4)c3Cl)c2Cl)ccc1CNC[C@@H]1CCC(=O)N1] | 0.0089 |
| | (S)-8-(2,2′-dichloro-3′-(6-methoxy-5-((((5-oxopyrrolidin-2-yl)methyl)amino)methyl)pyridin-2-yl)-[1,1′-biphenyl]-3-yl)-3-(((5-oxo-6- | |
| | azaspiro[3.4]octan-2-yl)amino)methyl)-4H-pyrido[1,2-a]pyrimidin-4-one | |
| | diastereoisomer II | |
| 252 | 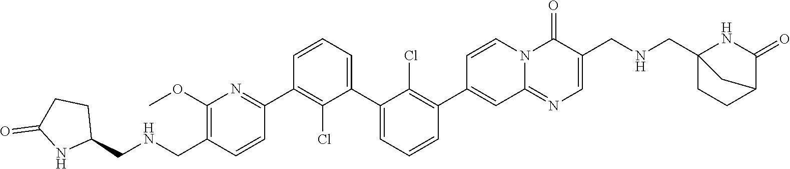 [SMILES: COc1nc(-c2cccc(-c3cccc(-c4ccn5c(=O)c(CNCC67CCC(C6)C(=O)N7)cnc5c4)c3Cl)c2Cl)ccc1CNC[C@@H]1CCC(=O)N1] | 0.0047 |
| | 8-(2,2′-dichloro-3′-(6-methoxy-5-(((((S)-5-oxopyrrolidin-2-yl)methyl)amino)methyl)pyridin-2-yl)-[1,1′-biphenyl]-3-yl)-3-((((3-oxo-2- | |
| | azabicyclo[2.2.1]heptan-1-yl)methyl)amino)methyl)-4H-pyrido[1,2-a]pyrimidin-4-one | |
| 253 | 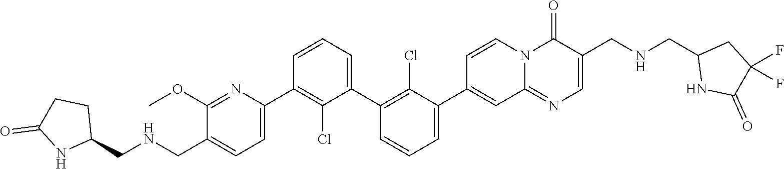 [SMILES: COc1nc(-c2cccc(-c3cccc(-c4ccn5c(=O)c(CNCC6CC(F)(F)C(=O)N6)cnc5c4)c3Cl)c2Cl)ccc1CNC[C@@H]1CCC(=O)N1] | 0.0057 |
| | 8-(2,2′-dichloro-3′-(6-methoxy-5-(((((S)-5-oxopyrrolidin-2-yl)methyl)amino)methyl)pyridin-2-yl)-[1,1′-biphenyl]-3-yl)-3-((((4,4- | |
| | difluoro-5-oxopyrrolidin-2-yl)methyl)amino)methyl)-4H-pyrido[1,2-a]pyrimidin-4-one | |
| | mixture of 2 diastereoisomers | |
| 254 | 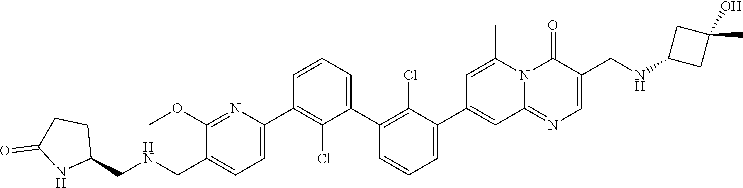 [SMILES: COc1nc(-c2cccc(-c3cccc(-c4cc(C)n5c(=O)c(CN[C@H]6C[C@@](C)(O)C6)cnc5c4)c3Cl)c2Cl)ccc1CNC[C@@H]1CCC(=O)N1] | 0.061 |
| | 8-(2,2′-dichloro-3′-(6-methoxy-5-(((((S)-5-oxopyrrolidin-2-yl)methyl)amino)methyl)pyridin-2-yl)-[1,1′-biphenyl]-3-yl)-3- | |
| | ((((1s,3s)-3-hydroxy-3-methylcyclobutyl)amino)methyl)-6-methyl-4H-pyrido[1,2-a]pyrimidin-4-one | |
| 255 | 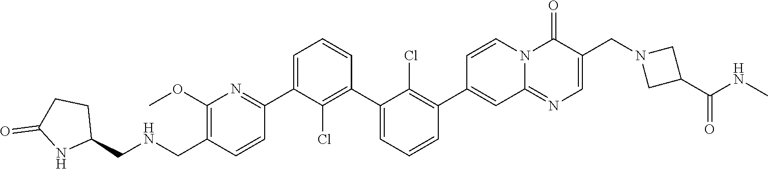 [SMILES: CNC(=O)C1CN(Cc2cnc3cc(-c4cccc(-c5cccc(-c6ccc(CNC[C@@H]7CCC(=O)N7)c(OC)n6)c5Cl)c4Cl)ccn3c2=O)C1] | 0.0001 |
| | (S)-1-((8-(2,2′-dichloro-3′-(6-methoxy-5-((((5-oxopyrrolidin-2-yl)methyl)amino)methyl)pyridin-2-yl)-[1,1′-biphenyl]-3-yl)-4-oxo- | |
| | 4H-pyrido[1,2-a]pyrimidin-3-yl)methyl)-N-methylazetidine-3-carboxamide | |
| 256 | 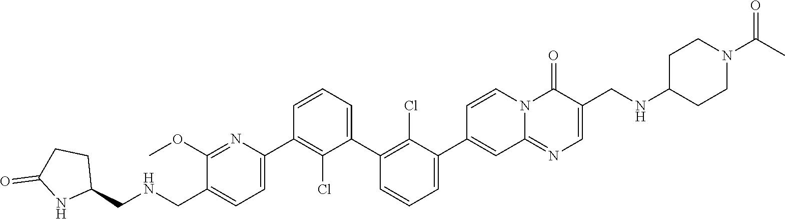 [SMILES: COc1nc(-c2cccc(-c3cccc(-c4ccn5c(=O)c(CNC6CCN(C(C)=O)CC6)cnc5c4)c3Cl)c2Cl)ccc1CNC[C@@H]1CCC(=O)N1] | 0.0001 |
| | (S)-3-(((1-acetylpiperidin-4-yl)amino)methyl)-8-(2,2′-dichloro-3′-(6-methoxy-5-((((5-oxopyrrolidin-2-yl)methyl)amino)methyl)pyridin- | |
| | 2-yl)-[1,1′-biphenyl]-3-yl)-4H-pyrido[1,2-a]pyrimidin-4-one | |
| 257 | 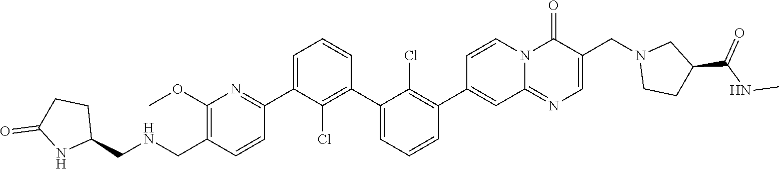 [SMILES: CNC(=O)[C@H]1CCN(Cc2cnc3cc(-c4cccc(-c5cccc(-c6ccc(CNC[C@@H]7CCC(=O)N7)c(OC)n6)c5Cl)c4Cl)ccn3c2=O)C1] | 0.0019 |
| | (S)-1-((8-(2,2′-dichloro-3′-(6-methoxy-5-(((((S)-5-oxopyrrolidin-2-yl)methyl)amino)methyl)pyridin-2-yl)-[1,1′-biphenyl]-3-yl)-4- | |
| | oxo-4H-pyrido[1,2-a]pyrimidin-3-yl)methyl)-N-methylpyrrolidine-3-carboxamide | |
| 258 | 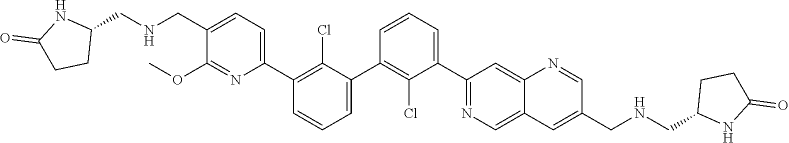 [SMILES: COc1nc(-c2cccc(-c3cccc(-c4cc5ncc(CNC[C@@H]6CCC(=O)N6)cc5cn4)c3Cl)c2Cl)ccc1CNC[C@@H]1CCC(=O)N1] | 0.0001 |
| | (S)-5-((((6-(2,2′-dichloro-3′-(3-(((((S)-5-oxopyrrolidin-2-yl)methyl)amino)methyl)-1,6-naphthyridin-7-yl)-[1,1′-biphenyl]-3-yl)-2- | |
| | methoxypyridin-3-yl)methyl)amino)methyl)pyrrolidin-2-one | |
| 259 | 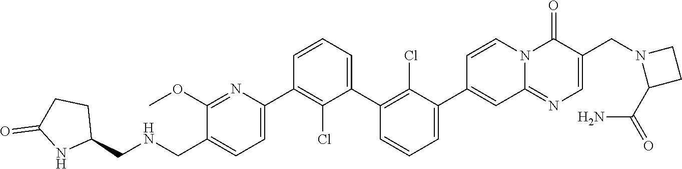 [SMILES: COc1nc(-c2cccc(-c3cccc(-c4ccn5c(=O)c(CN6CCC6C(N)=O)cnc5c4)c3Cl)c2Cl)ccc1CNC[C@@H]1CCC(=O)N1] | 0.0023 |
| | 1-((8-(2,2′-dichloro-3′-(6-methoxy-5-(((((S)-5-oxopyrrolidin-2-yl)methyl)amino)methyl)pyridin-2-yl)-[1,1′-biphenyl]- | |
| | 3-yl)-4-oxo-4H-pyrido[1,2-a]pyrimidin-3-yl)methyl)azetidine-2-carboxamide | |
| | mixture of 2 diastereoisomers | |
| 260 | 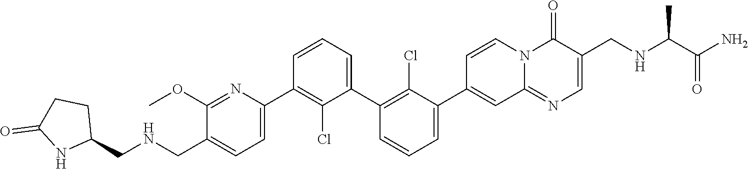 [SMILES: COc1nc(-c2cccc(-c3cccc(-c4ccn5c(=O)c(CN[C@@H](C)C(N)=O)cnc5c4)c3Cl)c2Cl)ccc1CNC[C@@H]1CCC(=O)N1] | 0.0001 |
| | (S)-2-(((8-(2,2′-dichloro-3′-(6-methoxy-5-(((((S)-5-oxopyrrolidin-2-yl)methyl)amino)methyl)pyridin-2-yl)-[1,1′-biphenyl]-3- | |
| | yl)-4-oxo-4H-pyrido[1,2-a]pyrimidin-3-yl)methyl)amino)propanamide | |
| 261 | 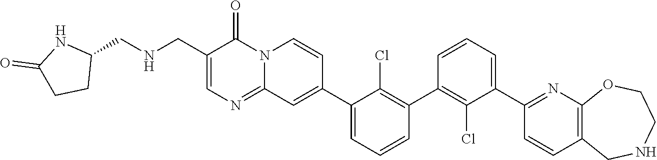 [SMILES: O=C1CC[C@@H](CNCc2cnc3cc(-c4cccc(-c5cccc(-c6ccc7c(n6)OCCNC7)c5Cl)c4Cl)ccn3c2=O)N1] | 0.25 |
| | (S)-8-(2,2′-dichloro-3′-(2,3,4,5-tetrahydropyrido[3,2-f][1,4]oxazepin-8-yl)-[1,1′-biphenyl]-3-yl)-3-((((5- | |
| | oxopyrrolidin-2-yl)methyl)amino)methyl)-4H-pyrido[1,2-a]pyrimidin-4-one | |
| 262 | 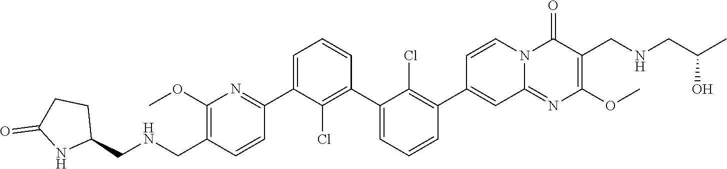 [SMILES: COc1nc(-c2cccc(-c3cccc(-c4ccn5c(=O)c(CNC[C@H](C)O)c(OC)nc5c4)c3Cl)c2Cl)ccc1CNC[C@@H]1CCC(=O)N1] | 0.0061 |
| | 8-(2,2′-dichloro-3′-(6-methoxy-5-(((((S)-5-oxopyrrolidin-2-yl)methyl)amino)methyl)pyridin-2-yl)-[1,1′-biphenyl]-3-yl)-3- | |
| | ((((S)-2-hydroxypropyl)amino)methyl)-2-methoxy-4H-pyrido[1,2-a]pyrimidin-4-one | |
| 263 | 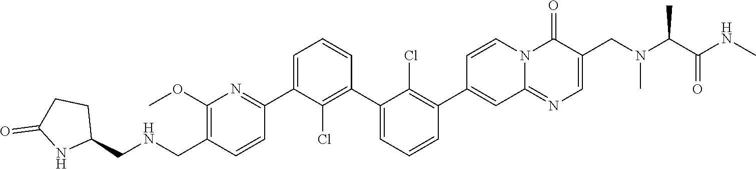 [SMILES: CNC(=O)[C@H](C)N(C)Cc1cnc2cc(-c3cccc(-c4cccc(-c5ccc(CNC[C@@H]6CCC(=O)N6)c(OC)n5)c4Cl)c3Cl)ccn2c1=O] | 0.0006 |
| | (S)-2-(((8-(2,2′-dichloro-3′-(6-methoxy-5-(((((S)-5-oxopyrrolidin-2-yl)methyl)amino)methyl)pyridin-2-yl)-[1,1′-biphenyl]-3-yl)- | |
| | 4-oxo-4H-pyrido[1,2-a]pyrimidin-3-yl)methyl)(methyl)amino)-N-methylpropanamide | |
| 264 | 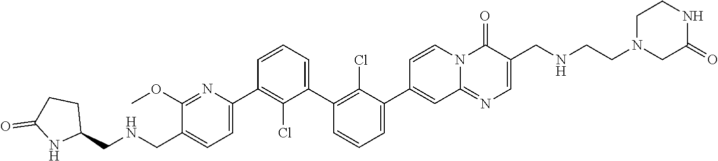 [SMILES: COc1nc(-c2cccc(-c3cccc(-c4ccn5c(=O)c(CNCCN6CCNC(=O)C6)cnc5c4)c3Cl)c2Cl)ccc1CNC[C@@H]1CCC(=O)N1] | 0.0001 |
| | (S)-8-(2,2′-dichloro-3′-(6-methoxy-5-((((5-oxopyrrolidin-2-yl)methyl)amino)methyl)pyridin-2-yl)-[1,1′-biphenyl]-3-yl)-3- | |
| | (((2-(3-oxopiperazin-1-yl)ethyl)amino)methyl)-4H-pyrido[1,2-a]pyrimidin-4-one | |
| 265 | 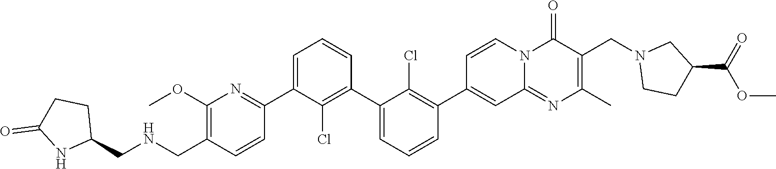 [SMILES: COC(=O)[C@H]1CCN(Cc2c(C)nc3cc(-c4cccc(-c5cccc(-c6ccc(CNC[C@@H]7CCC(=O)N7)c(OC)n6)c5Cl)c4Cl)ccn3c2=O)C1] | 0.0007 |
| | methyl (S)-1-((8-(2,2′-dichloro-3′-(6-methoxy-5-(((((S)-5-oxopyrrolidin-2-yl)methyl)amino)methyl)pyridin-2-yl)-[1,1′-biphenyl]-3- | |
| | yl)-2-methyl-4-oxo-4H-pyrido[1,2-a]pyrimidin-3-yl)methyl)pyrrolidine-3-carboxylate | |
| 266 | 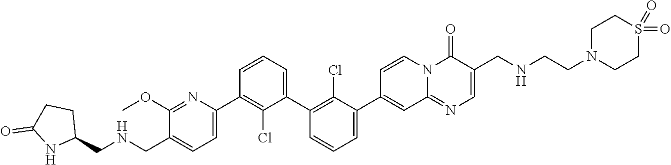 [SMILES: COc1nc(-c2cccc(-c3cccc(-c4ccn5c(=O)c(CNCCN6CCS(=O)(=O)CC6)cnc5c4)c3Cl)c2Cl)ccc1CNC[C@@H]1CCC(=O)N1] | 0.0172 |
| | (S)-8-(2,2′-dichloro-3′-(6-methoxy-5-((((5-oxopyrrolidin-2-yl)methyl)amino)methyl)pyridin-2-yl)-[1,1′-biphenyl]- | |
| | 3-yl)-3-(((2-(1,1-dioxidothiomorpholino)ethyl)amino)methyl)-4H-pyrido[1,2-a]pyrimidin-4-one | |
| 267 | 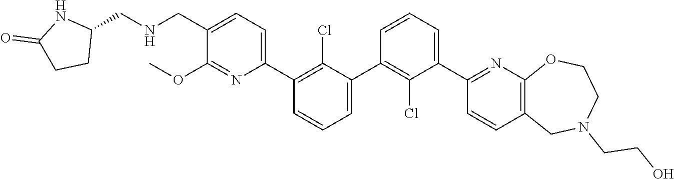 [SMILES: COc1nc(-c2cccc(-c3cccc(-c4ccc5c(n4)OCCN(CCO)C5)c3Cl)c2Cl)ccc1CNC[C@@H]1CCC(=O)N1] | 0.0002 |
| | (S)-5-((((6-(2,2′-dichloro-3′-(4-(2-hydroxyethyl)-2,3,4,5-tetrahydropyrido[3,2-f][1,4]oxazepin-8-yl)-[1,1′-biphenyl]- | |
| | 3-yl)-2-methoxypyridin-3-yl)methyl)amino)methyl)pyrrolidin-2-one | |
| 268 | 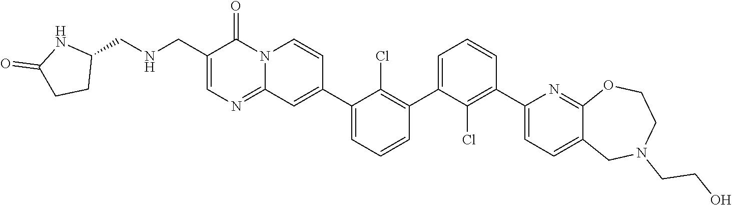 [SMILES: O=C1CC[C@@H](CNCc2cnc3cc(-c4cccc(-c5cccc(-c6ccc7c(n6)OCCN(CCO)C7)c5Cl)c4Cl)ccn3c2=O)N1] | 0.71 |
| | (S)-8-(2,2′-dichloro-3′-(4-(2-hydroxyethyl)-2,3,4,5-tetrahydropyrido[3,2-f][1,4]oxazepin-8-yl)-[1,1′-biphenyl]-3-yl)-3-((((5- | |
| | oxopyrrolidin-2-yl)methyl)amino)methyl)-4H-pyrido[1,2-a]pyrimidin-4-one | |
| 269 | 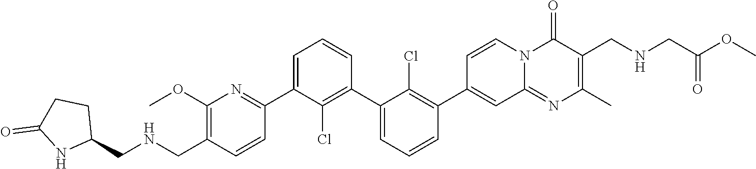 [SMILES: COC(=O)CNCc1c(C)nc2cc(-c3cccc(-c4cccc(-c5ccc(CNC[C@@H]6CCC(=O)N6)c(OC)n5)c4Cl)c3Cl)ccn2c1=O] | 0.0010 |
| | methyl (S)-((8-(2,2′-dichloro-3′-(6-methoxy-5-((((5-oxopyrrolidin-2-yl)methyl)amino)methyl)pyridin-2-yl)-[1,1′-biphenyl]-3-yl)- | |
| | 2-methyl-4-oxo-4H-pyrido[1,2-a]pyrimidin-3-yl)methyl)glycinate | |
| 270 | 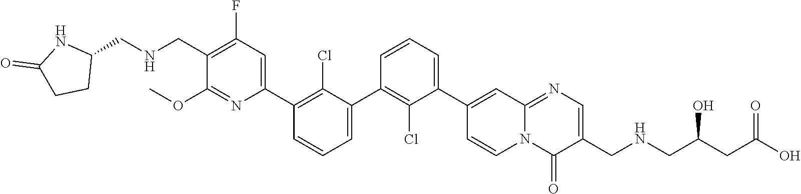 [SMILES: COc1nc(-c2cccc(-c3cccc(-c4ccn5c(=O)c(CNC[C@@H](O)CC(=O)O)cnc5c4)c3Cl)c2Cl)cc(F)c1CNC[C@@H]1CCC(=O)N1] | 0.0001 |
| | (S)-4-(((8-(2,2′-dichloro-3′-(4-fluoro-6-methoxy-5-(((((S)-5-oxopyrrolidin-2-yl)methyl)amino)methyl)pyridin-2-yl)-[1,1′-biphenyl]- | |
| | 3-yl)-4-oxo-4H-pyrido[1,2-a]pyrimidin-3-yl)methyl)amino)-3-hydroxybutanoic acid | |
| 271 | 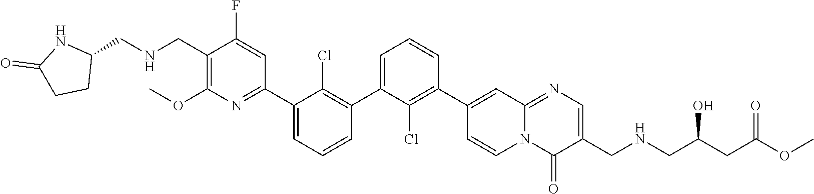 [SMILES: COC(=O)C[C@H](O)CNCc1cnc2cc(-c3cccc(-c4cccc(-c5cc(F)c(CNC[C@@H]6CCC(=O)N6)c(OC)n5)c4Cl)c3Cl)ccn2c1=O] | 0.0001 |
| | methyl (S)-4-(((8-(2,2′-dichloro-3′-(4-fluoro-6-methoxy-5-(((((S)-5-oxopyrrolidin-2-yl)methyl)amino)methyl)pyridin-2-yl)-[1,1′-biphenyl]- | |
| | 3-yl)-4-oxo-4H-pyrido[1,2-a]pyrimidin-3-yl)methyl)amino)-3-hydroxybutanoate | |
| 272 | 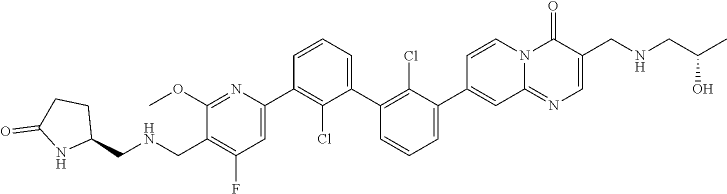 [SMILES: COc1nc(-c2cccc(-c3cccc(-c4ccn5c(=O)c(CNC[C@H](C)O)cnc5c4)c3Cl)c2Cl)cc(F)c1CNC[C@@H]1CCC(=O)N1] | 0.0002 |
| | 8-(2,2′-dichloro-3′-(4-fluoro-6-methoxy-5-(((((S)-5-oxopyrrolidin-2-yl)methyl)amino)methyl)pyridin-2-yl)-[1,1′-biphenyl]- | |
| | 3-yl)-3-((((S)-2-hydroxypropyl)amino)methyl)-4H-pyrido[1,2-a]pyrimidin-4-one | |
| 273 | 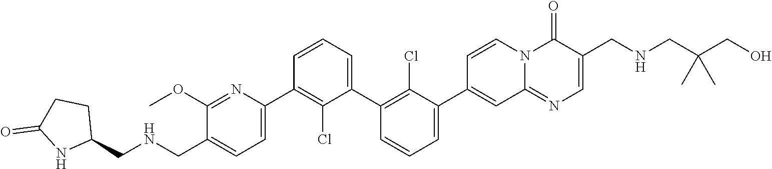 [SMILES: COc1nc(-c2cccc(-c3cccc(-c4ccn5c(=O)c(CNCC(C)(C)CO)cnc5c4)c3Cl)c2Cl)ccc1CNC[C@@H]1CCC(=O)N1] | 0.0042 |
| | (S)-8-(2,2′-dichloro-3′-(6-methoxy-5-((((5-oxopyrrolidin-2-yl)methyl)amino)methyl)pyridin-2-yl)-[1,1′-biphenyl]-3-yl)-3-(((3- | |
| | hydroxy-2,2-dimethylpropyl)amino)methyl)-4H-pyrido[1,2-a]pyrimidin-4-one | |
| 274 | 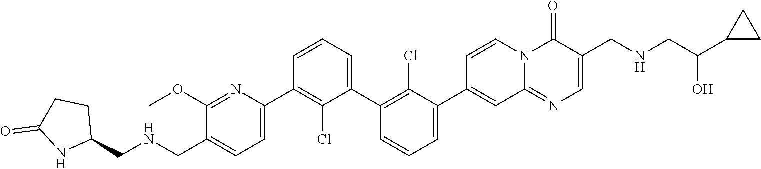 [SMILES: COc1nc(-c2cccc(-c3cccc(-c4ccn5c(=O)c(CNCC(O)C6CC6)cnc5c4)c3Cl)c2Cl)ccc1CNC[C@@H]1CCC(=O)N1] | 0.065 |
| | 3-(((2-cyclopropyl-2-hydroxyethyl)amino)methyl)-8-(2,2′-dichloro-3′-(6-methoxy-5-(((((S)-5-oxopyrrolidin-2- | |
| | yl)methyl)amino)methyl)pyridin-2-yl)-[1,1′-biphenyl]-3-yl)-4H-pyrido[1,2-a]pyrimidin-4-one | |
| | mixture of 2 diastereoisomers | |
| 275 | 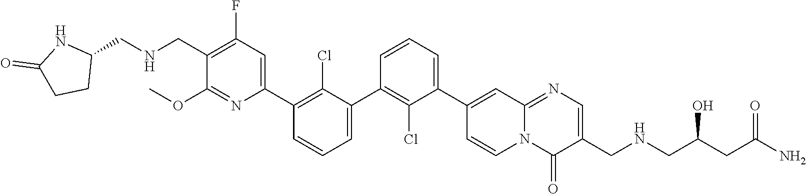 [SMILES: COc1nc(-c2cccc(-c3cccc(-c4ccn5c(=O)c(CNC[C@@H](O)CC(N)=O)cnc5c4)c3Cl)c2Cl)cc(F)c1CNC[C@@H]1CCC(=O)N1] | 0.0001 |
| | (S)-4-(((8-(2,2′-dichloro-3′-(4-fluoro-6-methoxy-5-(((((S)-5-oxopyrrolidin-2-yl)methyl)amino)methyl)pyridin-2-yl)-[1,1′-biphenyl]-3-yl)- | |
| | 4-oxo-4H-pyrido[1,2-a]pyrimidin-3-yl)methyl)amino)-3-hydroxybutanamide | |
| 276 | 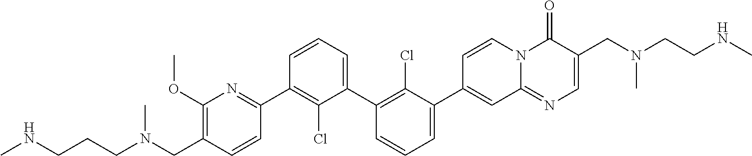 | 0.090 |
| | 8-(2,2′-dichloro-3′-(6-methoxy-5-((methyl(3-(methylamino)propyl)amino)methyl)pyridin-2-yl)-[1,1′-biphenyl]-3-yl)-3- | |
| | ((methyl(2-(methylamino)ethyl)amino)methyl)-4H-pyrido[1,2-a]pyrimidin-4-one | |
| 277 | 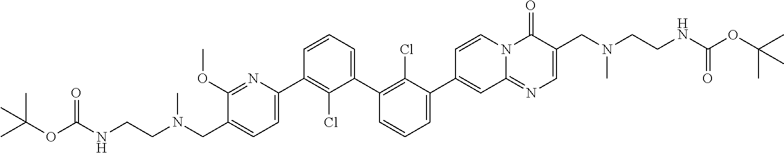 [SMILES: COc1nc(-c2cccc(-c3cccc(-c4ccn5c(=O)c(CN(C)CCNC(=O)OC(C)(C)C)cnc5c4)c3Cl)c2Cl)ccc1CN(C)CCNC(=O)OC(C)(C)C] | 0.20 |
| | tert-butyl (2-(((6-(3′-(3-(((2-((tert-butoxycarbonyl)amino)ethyl)(methyl)amino)methyl)-4-oxo-4H-pyrido[1,2-a]pyrimidin-8-yl)-2,2′- | |
| | dichloro-[1,1′-biphenyl]-3-yl)-2-methoxypyridin-3-yl)methyl)(methyl)amino)ethyl)carbamate | |
| 278 | 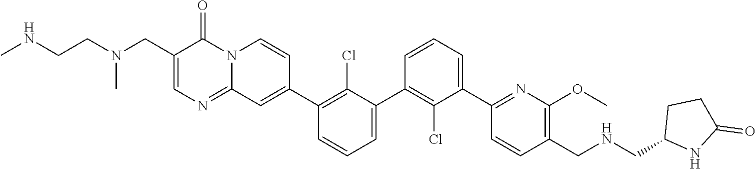 [SMILES: CNCCN(C)Cc1cnc2cc(-c3cccc(-c4cccc(-c5ccc(CNC[C@@H]6CCC(=O)N6)c(OC)n5)c4Cl)c3Cl)ccn2c1=O] | 0.0001 |
| | (S)-8-(2,2′-dichloro-3′-(6-methoxy-5-((((5-oxopyrrolidin-2-yl)methyl)amino)methyl)pyridin-2-yl)-[1,1′-biphenyl]-3-yl)-3- | |
| | ((methyl(2-(methylamino)ethyl)amino)methyl)-4H-pyrido[1,2-a]pyrimidin-4-one | |
| 279 | 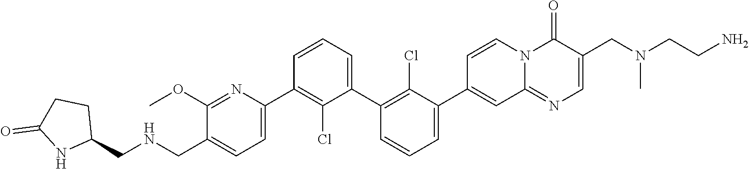 [SMILES: COc1nc(-c2cccc(-c3cccc(-c4ccn5c(=O)c(CN(C)CCN)cnc5c4)c3Cl)c2Cl)ccc1CNC[C@@H]1CCC(=O)N1] | 0.0001 |
| | (S)-3-(((2-aminoethyl)(methyl)amino)methyl)-8-(2,2′-dichloro-3′-(6-methoxy-5-((((5-oxopyrrolidin-2- | |
| | yl)methyl)amino)methyl)pyridin-2-yl)-[1,1′-biphenyl]-3-yl)-4H-pyrido[1,2-a]pyrimidin-4-one | |
| 280 | 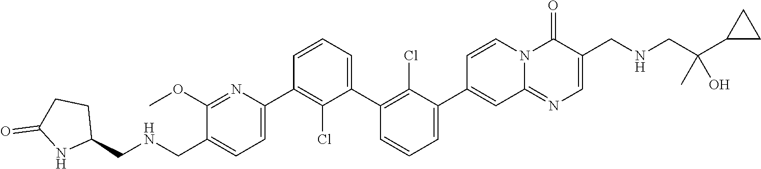 [SMILES: COc1nc(-c2cccc(-c3cccc(-c4ccn5c(=O)c(CNCC(C)(O)C6CC6)cnc5c4)c3Cl)c2Cl)ccc1CNC[C@@H]1CCC(=O)N1] | 0.037 |
| | 3-(((2-cyclopropyl-2-hydroxypropyl)amino)methyl)-8-(2,2′-dichloro-3′-(6-methoxy-5-(((((S)-5-oxopyrrolidin-2- | |
| | yl)methyl)amino)methyl)pyridin-2-yl)-[1,1′-biphenyl]-3-yl)-4H-pyrido[1,2-a]pyrimidin-4-one | |
| | mixture of 2 diastereoisomers | |
| 281 | 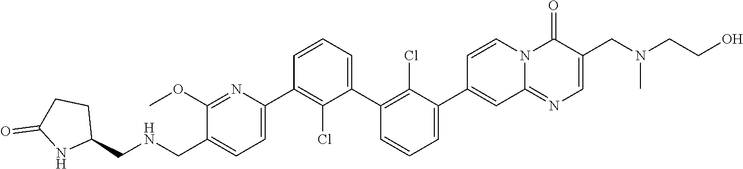 [SMILES: COc1nc(-c2cccc(-c3cccc(-c4ccn5c(=O)c(CN(C)CCO)cnc5c4)c3Cl)c2Cl)ccc1CNC[C@@H]1CCC(=O)N1] | 0.0001 |
| | (S)-8-(2,2′-dichloro-3′-(6-methoxy-5-((((5-oxopyrrolidin-2-yl)methyl)amino)methyl)pyridin-2-yl)-[1,1′-biphenyl]-3-yl)-3-(((2- | |
| | hydroxyethyl)(methyl)amino)methyl)-4H-pyrido[1,2-a]pyrimidin-4-one | |
| 282 | 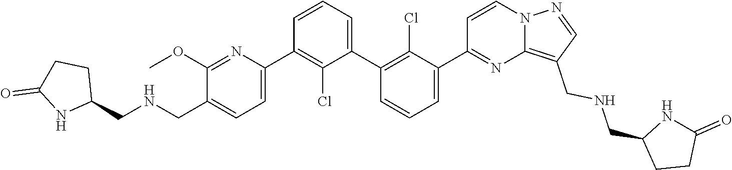 [SMILES: COc1nc(-c2cccc(-c3cccc(-c4ccn5ncc(CNC[C@@H]6CCC(=O)N6)c5n4)c3Cl)c2Cl)ccc1CNC[C@@H]1CCC(=O)N1] | 0.0087 |
| | (S)-5-((((6-(2,2′-dichloro-3′-(3-(((((S)-5-oxopyrrolidin-2-yl)methyl)amino)methyl)pyrazolo[1,5-a]pyrimidin-5-yl)-[1,1′- | |
| | biphenyl]-3-yl)-2-methoxypyridin-3-yl)methyl)amino)methyl)pyrrolidin-2-one | |
| 283 | 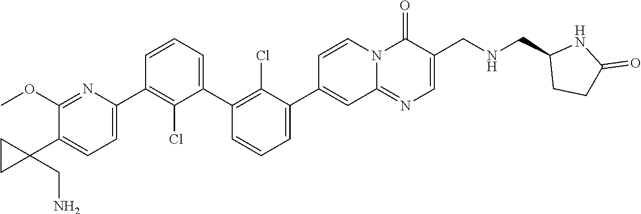 [SMILES: COc1nc(-c2cccc(-c3cccc(-c4ccn5c(=O)c(CNC[C@@H]6CCC(=O)N6)cnc5c4)c3Cl)c2Cl)ccc1C1(CN)CC1] | 0.15 |
| | (S)-8-(3′-(5-(1-(aminomethyl)cyclopropyl)-6-methoxypyridin-2-yl)-2,2′-dichloro-[1,1′-biphenyl]-3-yl)-3-((((5- | |
| | oxopyrrolidin-2-yl)methyl)amino)methyl)-4H-pyrido[1,2-a]pyrimidin-4-one | |
| 284 | 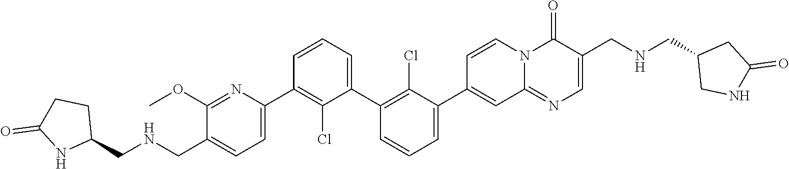 [SMILES: COc1nc(-c2cccc(-c3cccc(-c4ccn5c(=O)c(CNC[C@H]6CNC(=O)C6)cnc5c4)c3Cl)c2Cl)ccc1CNC[C@@H]1CCC(=O)N1] | 0.0001 |
| | 8-(2,2′-dichloro-3′-(6-methoxy-5-(((((S)-5-oxopyrrolidin-2-yl)methyl)amino)methyl)pyridin-2-yl)-[1,1′-biphenyl]-3-yl)-3-(((((S)-5- | |
| | oxopyrrolidin-3-yl)methyl)amino)methyl)-4H-pyrido[1,2-a]pyrimidin-4-one | |
| 288 | 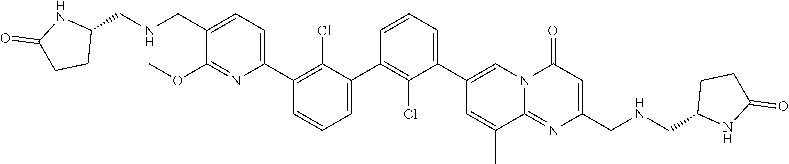 [SMILES: COc1nc(-c2cccc(-c3cccc(-c4cc(C)c5nc(CNC[C@@H]6CCC(=O)N6)cc(=O)n5c4)c3Cl)c2Cl)ccc1CNC[C@@H]1CCC(=O)N1] | 0.056 |
| | 7-(2,2′-dichloro-3′-(6-methoxy-5-(((((S)-5-oxopyrrolidin-2-yl)methyl)amino)methyl)pyridin-2-yl)-[1,1′-biphenyl]-3-yl)-9-methyl-2- | |
| | (((((S)-5-oxopyrrolidin-2-yl)methyl)amino)methyl)-4H-pyrido[1,2-a]pyrimidin-4-one | |
| 289 | 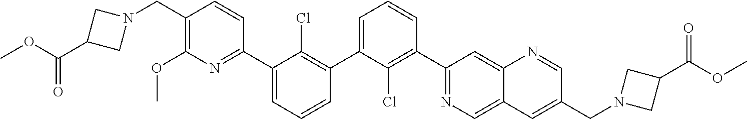 | 0.0090 |
| | methyl 1-((6-(2,2′-dichloro-3′-(3-((3-(methoxycarbonyl)azetidin-1-yl)methyl)-1,6-naphthyridin-7-yl)-[1,1′-biphenyl]-3-yl)-2- | |
| | methoxypyridin-3-yl)methyl)azetidine-3-carboxylate | |
| 290 | 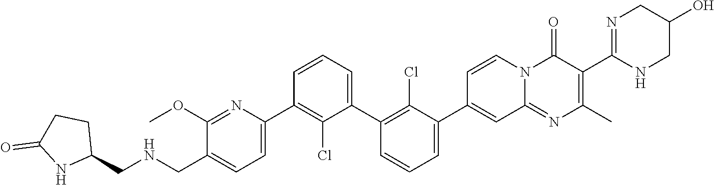 [SMILES: COc1nc(-c2cccc(-c3cccc(-c4ccn5c(=O)c(C6=NCC(O)CN6)c(C)nc5c4)c3Cl)c2Cl)ccc1CNC[C@@H]1CCC(=O)N1] | 0.0004 |
| | 8-(2,2′-dichloro-3′-(6-methoxy-5-(((((S)-5-oxopyrrolidin-2-yl)methyl)amino)methyl)pyridin-2-yl)-[1,1′-biphenyl]-3-yl)-3- | |
| | (5-hydroxy-1,4,5,6-tetrahydropyrimidin-2-yl)-2-methyl-4H-pyrido[1,2-a]pyrimidin-4-one | |
| 291 | 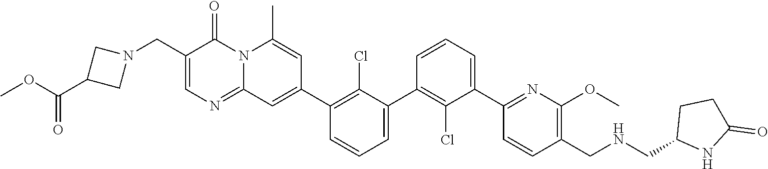 [SMILES: COC(=O)C1CN(Cc2cnc3cc(-c4cccc(-c5cccc(-c6ccc(CNC[C@@H]7CCC(=O)N7)c(OC)n6)c5Cl)c4Cl)cc(C)n3c2=O)C1] | 0.0001 |
| | methyl (S)-1-((8-(2,2′-dichloro-3′-(6-methoxy-5-((((5-oxopyrrolidin-2-yl)methyl)amino)methyl)pyridin-2-yl)-[1,1′-biphenyl]-3-yl)- | |
| | 6-methyl-4-oxo-4H-pyrido[1,2-a]pyrimidin-3-yl)methyl)azetidine-3-carboxylate | |
| 292 | 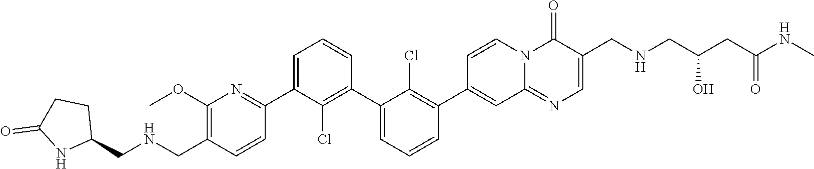 [SMILES: CNC(=O)C[C@H](O)CNCc1cnc2cc(-c3cccc(-c4cccc(-c5ccc(CNC[C@@H]6CCC(=O)N6)c(OC)n5)c4Cl)c3Cl)ccn2c1=O] | 0.0001 |
| | (S)-4-(((8-(2,2′-dichloro-3′-(6-methoxy-5-(((((S)-5-oxopyrrolidin-2-yl)methyl)amino)methyl)pyridin-2-yl)-[1,1′-biphenyl]-3-yl)-4-oxo-4H- | |
| | pyrido[1,2-a]pyrimidin-3-yl)methyl)amino)-3-hydroxy-N-methylbutanamide | |
| 293 | 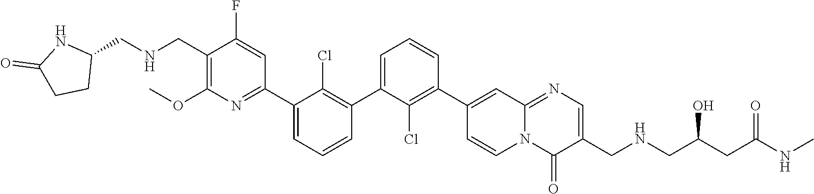 [SMILES: CNC(=O)C[C@H](O)CNCc1cnc2cc(-c3cccc(-c4cccc(-c5cc(F)c(CNC[C@@H]6CCC(=O)N6)c(OC)n5)c4Cl)c3Cl)ccn2c1=O] | 0.0001 |
| | (S)-4-(((8-(2,2′-dichloro-3′-(4-fluoro-6-methoxy-5-(((((S)-5-oxopyrrolidin-2-yl)methyl)amino)methyl)pyridin-2-yl)-[1,1′-biphenyl]-3-yl)- | |
| | 4-oxo-4H-pyrido[1,2-a]pyrimidin-3-yl)methyl)amino)-3-hydroxy-N-methylbutanamide | |
| 294 | 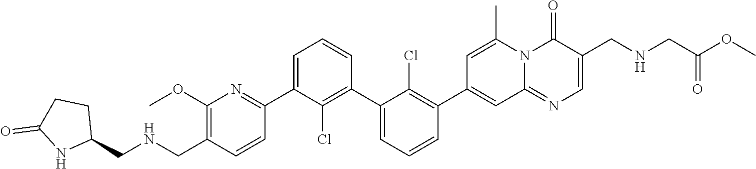 [SMILES: COC(=O)CNCc1cnc2cc(-c3cccc(-c4cccc(-c5ccc(CNC[C@@H]6CCC(=O)N6)c(OC)n5)c4Cl)c3Cl)cc(C)n2c1=O] | 0.0004 |
| | methyl (S)-((8-(2,2′-dichloro-3′-(6-methoxy-5-((((5-oxopyrrolidin-2-yl)methyl)amino)methyl)pyridin-2-yl)-[1,1′-biphenyl]-3-yl)- | |
| | 6-methyl-4-oxo-4H-pyrido[1,2-a]pyrimidin-3-yl)methyl)glycinate | |
| 295 | 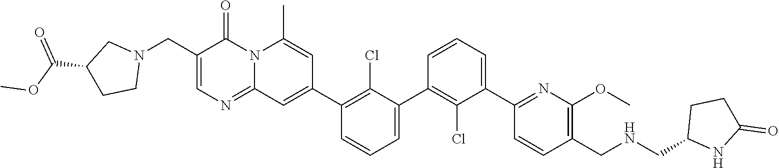 [SMILES: COC(=O)[C@H]1CCN(Cc2cnc3cc(-c4cccc(-c5cccc(-c6ccc(CNC[C@@H]7CCC(=O)N7)c(OC)n6)c5Cl)c4Cl)cc(C)n3c2=O)C1] | 0.0001 |
| | methyl (S)-1-((8-(2,2′-dichloro-3′-(6-methoxy-5-(((((S)-5-oxopyrrolidin-2-yl)methyl)amino)methyl)pyridin-2-yl)-[1,1′-biphenyl]-3- | |
| | yl)-6-methyl-4-oxo-4H-pyrido[1,2-a]pyrimidin-3-yl)methyl)pyrrolidine-3-carboxylate | |
| 296 | 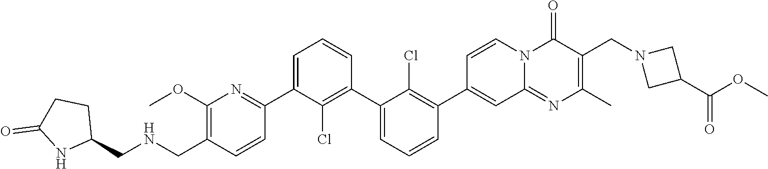 [SMILES: COC(=O)C1CN(Cc2c(C)nc3cc(-c4cccc(-c5cccc(-c6ccc(CNC[C@@H]7CCC(=O)N7)c(OC)n6)c5Cl)c4Cl)ccn3c2=O)C1] | 0.0005 |
| | methyl (S)-1-((8-(2,2′-dichloro-3′-(6-methoxy-5-((((5-oxopyrrolidin-2-yl)methyl)amino)methyl)pyridin-2-yl)-[1,1′-biphenyl]-3-yl)- | |
| | 2-methyl-4-oxo-4H-pyrido[1,2-a]pyrimidin-3-yl)methyl)azetidine-3-carboxylate | |
| 297 | 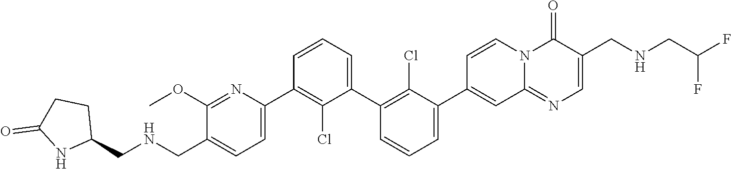 [SMILES: COc1nc(-c2cccc(-c3cccc(-c4ccn5c(=O)c(CNCC(F)F)cnc5c4)c3Cl)c2Cl)ccc1CNC[C@@H]1CCC(=O)N1] | 0.32 |
| | (S)-8-(2,2′-dichloro-3′-(6-methoxy-5-((((5-oxopyrrolidin-2-yl)methyl)amino)methyl)pyridin-2-yl)-[1,1′-biphenyl]-3-yl)-3- | |
| | (((2,2-difluoroethyl)amino)methyl)-4H-pyrido[1,2-a]pyrimidin-4-one | |
| 298 | 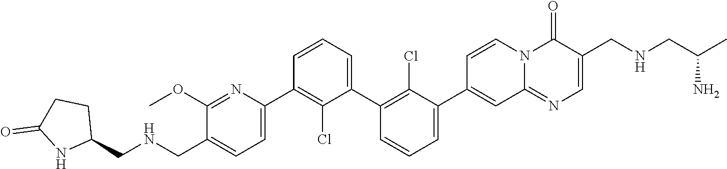 [SMILES: COc1nc(-c2cccc(-c3cccc(-c4ccn5c(=O)c(CNC[C@H](C)N)cnc5c4)c3Cl)c2Cl)ccc1CNC[C@@H]1CCC(=O)N1] | 0.025 |
| | 3-((((S)-2-aminopropyl)amino)methyl)-8-(2,2′-dichloro-3′-(6-methoxy-5-(((((S)-5-oxopyrrolidin-2- | |
| | yl)methyl)amino)methyl)pyridin-2-yl)-[1,1′-biphenyl]-3-yl)-4H-pyrido[1,2-a]pyrimidin-4-one | |
| 299 | 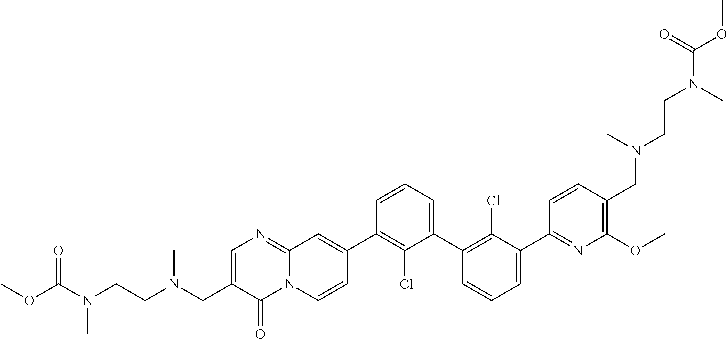 | 3.0 |
| | methyl (2-(((8-(2,2′-dichloro-3′-(6-methoxy-5-(((2-((methoxycarbonyl)(methyl)amino)ethyl)(methyl)amino)methyl)pyridin- | |
| | 2-yl)-[1,1′-biphenyl]-3-yl)-4-oxo-4H-pyrido[1,2-a]pyrimidin-3-yl)methyl)(methyl)amino)ethyl)(methyl)carbamate | |
| 300 | 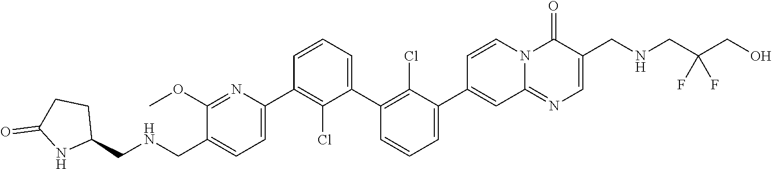 [SMILES: COc1nc(-c2cccc(-c3cccc(-c4ccn5c(=O)c(CNCC(F)(F)CO)cnc5c4)c3Cl)c2Cl)ccc1CNC[C@@H]1CCC(=O)N1] | 0.13 |
| | (S)-8-(2,2′-dichloro-3′-(6-methoxy-5-((((5-oxopyrrolidin-2-yl)methyl)amino)methyl)pyridin-2-yl)-[1,1′-biphenyl]-3-yl)-3-(((2,2- | |
| | difluoro-3-hydroxypropyl)amino)methyl)-4H-pyrido[1,2-a]pyrimidin-4-one | |
| 301 | 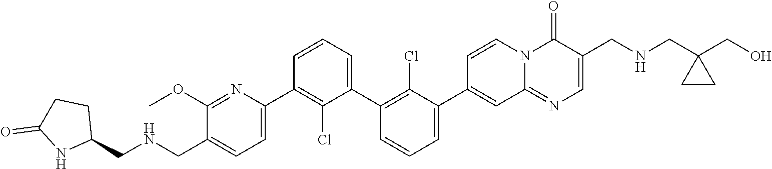 [SMILES: COc1nc(-c2cccc(-c3cccc(-c4ccn5c(=O)c(CNCC6(CO)CC6)cnc5c4)c3Cl)c2Cl)ccc1CNC[C@@H]1CCC(=O)N1] | 0.040 |
| | (S)-8-(2,2′-dichloro-3′-(6-methoxy-5-((((5-oxopyrrolidin-2-yl)methyl)amino)methyl)pyridin-2-yl)-[1,1′-biphenyl]-3-yl)-3-((((1- | |
| | (hydroxymethyl)cyclopropyl)methyl)amino)methyl)-4H-pyrido[1,2-a]pyrimidin-4-one | |
| 302 | 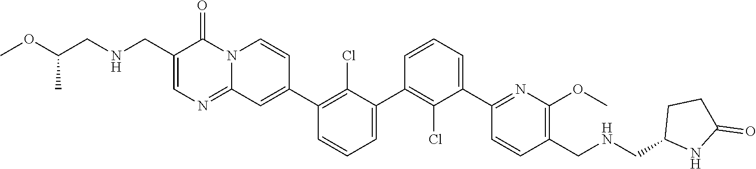 [SMILES: COc1nc(-c2cccc(-c3cccc(-c4ccn5c(=O)c(CNC[C@H](C)OC)cnc5c4)c3Cl)c2Cl)ccc1CNC[C@@H]1CCC(=O)N1] | 0.035 |
| | 8-(2,2′-dichloro-3′-(6-methoxy-5-(((((S)-5-oxopyrrolidin-2-yl)methyl)amino)methyl)pyridin-2-yl)-[1,1′-biphenyl]-3-yl)-3-((((S)- | |
| | 2-methoxypropyl)amino)methyl)-4H-pyrido[1,2-a]pyrimidin-4-one | |
| 303 | 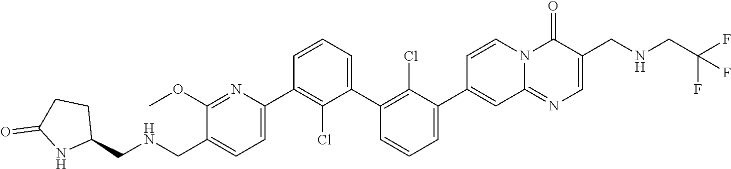 [SMILES: COc1nc(-c2cccc(-c3cccc(-c4ccn5c(=O)c(CNCC(F)(F)F)cnc5c4)c3Cl)c2Cl)ccc1CNC[C@@H]1CCC(=O)N1] | 0.0004 |
| | (S)-8-(2,2′-dichloro-3′-(6-methoxy-5-((((5-oxopyrrolidin-2-yl)methyl)amino)methyl)pyridin-2-yl)-[1,1′-biphenyl]-3-yl)-3- (((2,2,2-trifluoroethyl)amino)methyl)-4H-pyrido[1,2-a]pyrimidin-4-one | |
| 304 | 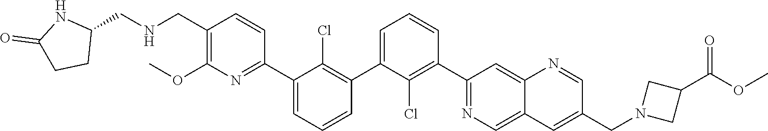 [SMILES: COC(=O)C1CN(Cc2cnc3cc(-c4cccc(-c5cccc(-c6ccc(CNC[C@@H]7CCC(=O)N7)c(OC)n6)c5Cl)c4Cl)ncc3c2)C1] | 0.0002 |
| | methyl (S)-1-((7-(2,2′-dichloro-3′-(6-methoxy-5-((((5-oxopyrrolidin-2-yl)methyl)amino)methyl)pyridin-2-yl)-[1,1′-biphenyl]-3-yl)- | |
| | 1,6-naphthyridin-3-yl)methyl)azetidine-3-carboxylate | |
| 305 | 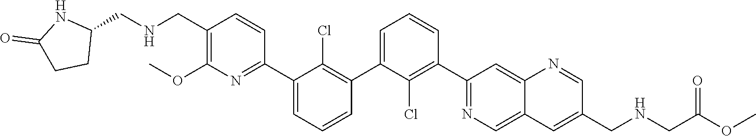 [SMILES: COC(=O)CNCc1cnc2cc(-c3cccc(-c4cccc(-c5ccc(CNC[C@@H]6CCC(=O)N6)c(OC)n5)c4Cl)c3Cl)ncc2c1] | 0.0004 |
| | methyl (S)-((7-(2,2′-dichloro-3′-(6-methoxy-5-((((5-oxopyrrolidin-2-yl)methyl)amino)methyl)pyridin-2-yl)-[1,1′-biphenyl]-3-yl)- | |
| | 1,6-naphthyridin-3-yl)methyl)glycinate | |
| 306 | 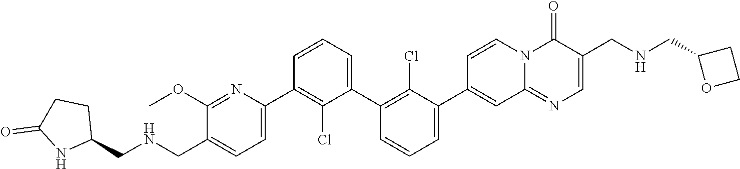 [SMILES: COc1nc(-c2cccc(-c3cccc(-c4ccn5c(=O)c(CNC[C@@H]6CCO6)cnc5c4)c3Cl)c2Cl)ccc1CNC[C@@H]1CCC(=O)N1] | 0.0048 |
| | 8-(2,2′-dichloro-3′-(6-methoxy-5-(((((S)-5-oxopyrrolidin-2-yl)methyl)amino)methyl)pyridin-2-yl)-[1,1′-biphenyl]-3-yl)-3- | |
| | (((((S)-oxetan-2-yl)methyl)amino)methyl)-4H-pyrido[1,2-a]pyrimidin-4-one | |
| 307 | 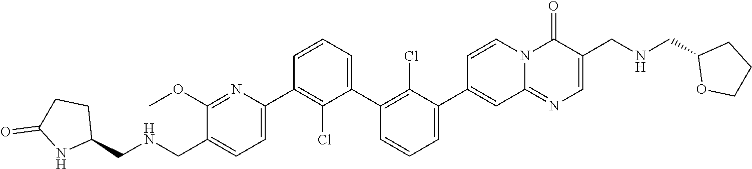 [SMILES: COc1nc(-c2cccc(-c3cccc(-c4ccn5c(=O)c(CNC[C@@H]6CCCO6)cnc5c4)c3Cl)c2Cl)ccc1CNC[C@@H]1CCC(=O)N1] | 0.037 |
| | 8-(2,2′-dichloro-3′-(6-methoxy-5-(((((S)-5-oxopyrrolidin-2-yl)methyl)amino)methyl)pyridin-2-yl)-[1,1′-biphenyl]-3-yl)-3-(((((S)- | |
| | tetrahydrofuran-2-yl)methyl)amino)methyl)-4H-pyrido[1,2-a]pyrimidin-4-one | |
| 308 | 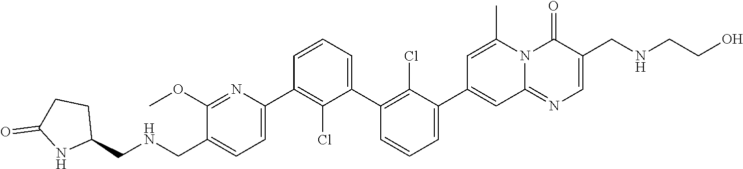 [SMILES: COc1nc(-c2cccc(-c3cccc(-c4cc(C)n5c(=O)c(CNCCO)cnc5c4)c3Cl)c2Cl)ccc1CNC[C@@H]1CCC(=O)N1] | 0.022 |
| | (S)-8-(2,2′-dichloro-3′-(6-methoxy-5-((((5-oxopyrrolidin-2-yl)methyl)amino)methyl)pyridin-2-yl)-[1,1′-biphenyl]-3-yl)-3-(((2- | |
| | hydroxyethyl)amino)methyl)-6-methyl-4H-pyrido[1,2-a]pyrimidin-4-one | |
| 309 | 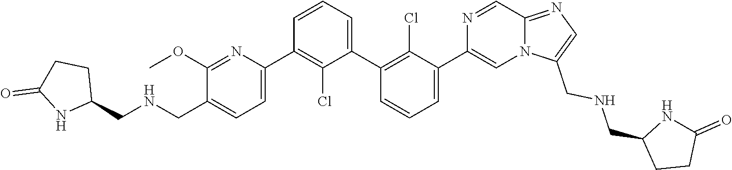 [SMILES: COc1nc(-c2cccc(-c3cccc(-c4cn5c(CNC[C@@H]6CCC(=O)N6)cnc5cn4)c3Cl)c2Cl)ccc1CNC[C@@H]1CCC(=O)N1] | 0.0003 |
| | (S)-5-((((6-(2,2′-dichloro-3′-(3-(((((S)-5-oxopyrrolidin-2-yl)methyl)amino)methyl)imidazo[1,2-a]pyrazin-6-yl)-[1,1′- | |
| | biphenyl]-3-yl)-2-methoxypyridin-3-yl)methyl)amino)methyl)pyrrolidin-2-one | |
| 310 | 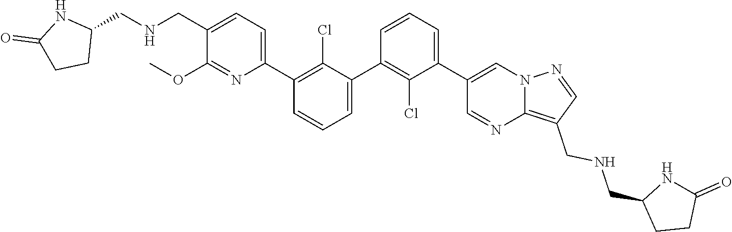 [SMILES: COc1nc(-c2cccc(-c3cccc(-c4cnc5c(CNC[C@@H]6CCC(=O)N6)cnn5c4)c3Cl)c2Cl)ccc1CNC[C@@H]1CCC(=O)N1] | 0.0002 |
| | (S)-5-((((6-(2,2′-dichloro-3′-(3-(((((S)-5-oxopyrrolidin-2-yl)methyl)amino)methyl)pyrazolo[1,5-a]pyrimidin-6-yl)-[1,1′- | |
| | biphenyl]-3-yl)-2-methoxypyridin-3-yl)methyl)amino)methyl)pyrrolidin-2-one | |
| 311 | 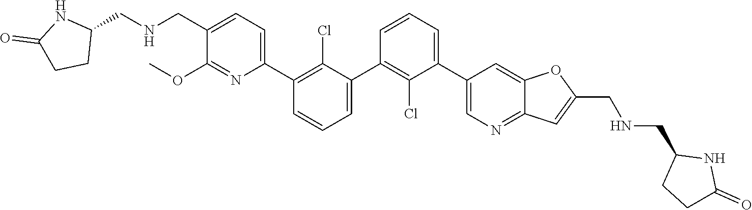 [SMILES: COc1nc(-c2cccc(-c3cccc(-c4cnc5cc(CNC[C@@H]6CCC(=O)N6)oc5c4)c3Cl)c2Cl)ccc1CNC[C@@H]1CCC(=O)N1] | 0.0050 |
| | (S)-5-((((6-(2,2′-dichloro-3′-(2-(((((S)-5-oxopyrrolidin-2-yl)methyl)amino)methyl)furo[3,2-b]pyridin-6-yl)-[1,1′-biphenyl]-3-yl)- | |
| | 2-methoxypyridin-3-yl)methyl)amino)methyl)pyrrolidin-2-one | |
| 312 | 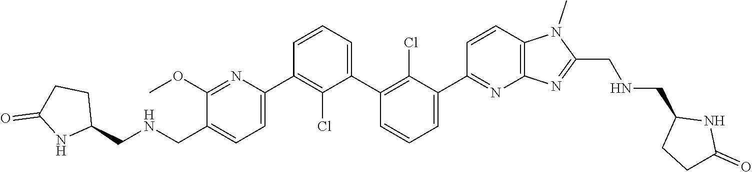 [SMILES: COc1nc(-c2cccc(-c3cccc(-c4ccc5c(n4)nc(CNC[C@@H]4CCC(=O)N4)n5C)c3Cl)c2Cl)ccc1CNC[C@@H]1CCC(=O)N1] | 0.0001 |
| | (S)-5-((((6-(2,2′-dichloro-3′-(1-methyl-2-(((((S)-5-oxopyrrolidin-2-yl)methyl)amino)methyl)-1H-imidazo[4,5-b]pyridin-5-yl)- | |
| | [1,1′-biphenyl]-3-yl)-2-methoxypyridin-3-yl)methyl)amino)methyl)pyrrolidin-2-one | |
| 313 | 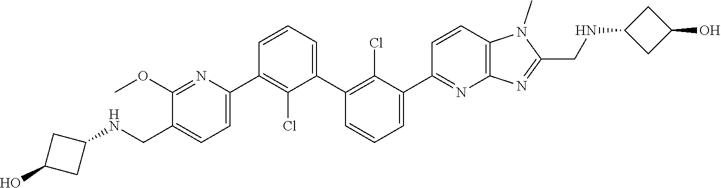 [SMILES: COc1nc(-c2cccc(-c3cccc(-c4ccc5c(n4)nc(CN[C@H]4C[C@H](O)C4)n5C)c3Cl)c2Cl)ccc1CN[C@H]1C[C@H](O)C1] | 0.0019 |
| | (1r,3r)-3-(((6-(2,2′-dichloro-3′-(2-((((1r,3r)-3-hydroxycyclobutyl)amino)methyl)-1-methyl-1H-imidazo[4,5-b]pyridin-5-yl)- | |
| | [1,1′-biphenyl]-3-yl)-2-methoxypyridin-3-yl)methyl)amino)cyclobutan-1-ol | |
| 314 | 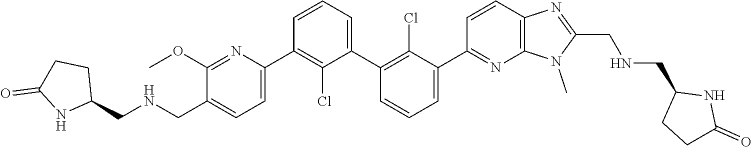 [SMILES: COc1nc(-c2cccc(-c3cccc(-c4ccc5nc(CNC[C@@H]6CCC(=O)N6)n(C)c5n4)c3Cl)c2Cl)ccc1CNC[C@@H]1CCC(=O)N1] | 0.0039 |
| | (S)-5-((((6-(2,2′-dichloro-3′-(3-methyl-2-(((((S)-5-oxopyrrolidin-2-yl)methyl)amino)methyl)-3H-imidazo[4,5-b]pyridin-5-yl)- | |
| | [1,1′-biphenyl]-3-yl)-2-methoxypyridin-3-yl)methyl)amino)methyl)pyrrolidin-2-one | |
| 315 | 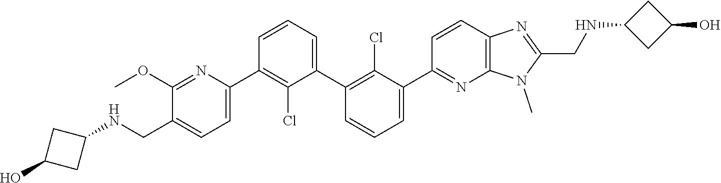 [SMILES: COc1nc(-c2cccc(-c3cccc(-c4ccc5nc(CN[C@H]6C[C@H](O)C6)n(C)c5n4)c3Cl)c2Cl)ccc1CN[C@H]1C[C@H](O)C1] | 0.0004 |
| | (1r,3r)-3-(((6-(2,2′-dichloro-3′-(2-((((1r,3r)-3-hydroxycyclobutyl)amino)methyl)-3-methyl-3H-imidazo[4,5-b]pyridin-5-yl)- | |
| | [1,1′-biphenyl]-3-yl)-2-methoxypyridin-3-yl)methyl)amino)cyclobutan-1-ol | |
| 316 | 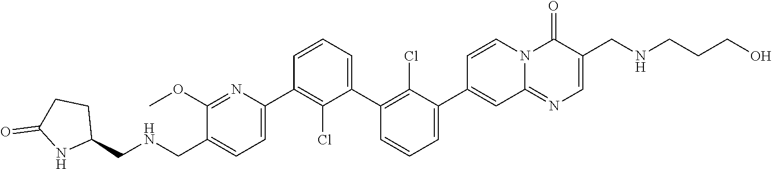 [SMILES: COc1nc(-c2cccc(-c3cccc(-c4ccn5c(=O)c(CNCCCO)cnc5c4)c3Cl)c2Cl)ccc1CNC[C@@H]1CCC(=O)N1] | 0.0003 |
| | (S)-8-(2,2′-dichloro-3′-(6-methoxy-5-((((5-oxopyrrolidin-2-yl)methyl)amino)methyl)pyridin-2-yl)-[1,1′-biphenyl]-3-yl)-3-(((3- | |
| | hydroxypropyl)amino)methyl)-4H-pyrido[1,2-a]pyrimidin-4-one | |
| 317 | 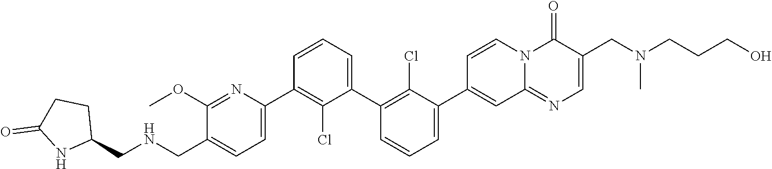 [SMILES: COc1nc(-c2cccc(-c3cccc(-c4ccn5c(=O)c(CN(C)CCCO)cnc5c4)c3Cl)c2Cl)ccc1CNC[C@@H]1CCC(=O)N1] | 0.0002 |
| | (S)-8-(2,2′-dichloro-3′-(6-methoxy-5-((((5-oxopyrrolidin-2-yl)methyl)amino)methyl)pyridin-2-yl)-[1,1′-biphenyl]-3-yl)-3-(((3- | |
| | hydroxypropyl)(methyl)amino)methyl)-4H-pyrido[1,2-a]pyrimidin-4-one | |
| 318 | 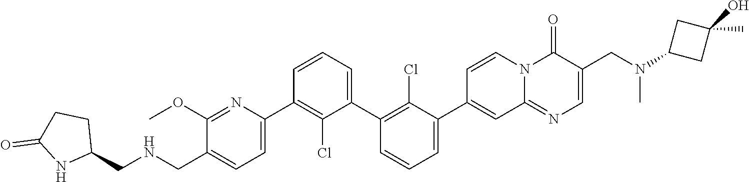 [SMILES: COc1nc(-c2cccc(-c3cccc(-c4ccn5c(=O)c(CN(C)[C@H]6C[C@](C)(O)C6)cnc5c4)c3Cl)c2Cl)ccc1CNC[C@@H]1CCC(=O)N1] | 0.0001 |
| | 8-(2,2′-dichloro-3′-(6-methoxy-5-(((((S)-5-oxopyrrolidin-2-yl)methyl)amino)methyl)pyridin-2-yl)-[1,1′-biphenyl]-3-yl)-3- | |
| | ((((1r,3r)-3-hydroxy-3-methylcyclobutyl)(methyl)amino)methyl)-4H-pyrido[1,2-a]pyrimidin-4-one | |
| 319 | 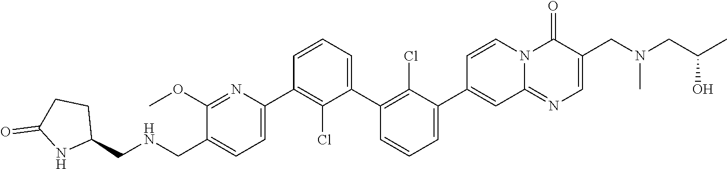 [SMILES: COc1nc(-c2cccc(-c3cccc(-c4ccn5c(=O)c(CN(C)C[C@H](C)O)cnc5c4)c3Cl)c2Cl)ccc1CNC[C@@H]1CCC(=O)N1] | 0.0001 |
| | 8-(2,2′-dichloro-3′-(6-methoxy-5-(((((S)-5-oxopyrrolidin-2-yl)methyl)amino)methyl)pyridin-2-yl)-[1,1′-biphenyl]-3-yl)-3- | |
| | ((((S)-2-hydroxypropyl)(methyl)amino)methyl)-4H-pyrido[1,2-a]pyrimidin-4-one | |
| 320 | 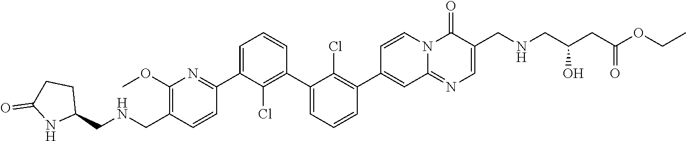 [SMILES: CCOC(=O)C[C@H](O)CNCc1cnc2cc(-c3cccc(-c4cccc(-c5ccc(CNC[C@@H]6CCC(=O)N6)c(OC)n5)c4Cl)c3Cl)ccn2c1=O] | 0.0000 |
| | ethyl (S)-4-(((8-(2,2′-dichloro-3′-(6-methoxy-5-(((((S)-5-oxopyrrolidin-2-yl)methyl)amino)methyl)pyridin-2-yl)- | |
| | [1,1′-biphenyl]-3-yl)-4-oxo-4H-pyrido[1,2-a]pyrimidin-3-yl)methyl)amino)-3-hydroxybutanoate | |
| 321 | 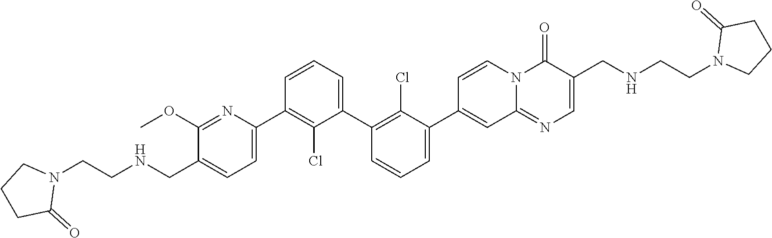 [SMILES: COc1nc(-c2cccc(-c3cccc(-c4ccn5c(=O)c(CNCCN6CCCC6=O)cnc5c4)c3Cl)c2Cl)ccc1CNCCN1CCCC1=O] | 0.0002 |
| | 8-(2,2′-dichloro-3′-(6-methoxy-5-(((2-(2-oxopyrrolidin-1-yl)ethyl)amino)methyl)pyridin-2-yl)-[1,1′-biphenyl]-3-yl)-3-(((2-(2- | |
| | oxopyrrolidin-1-yl)ethyl)amino)methyl)-4H-pyrido[1,2-a]pyrimidin-4-one | |
| 322 | 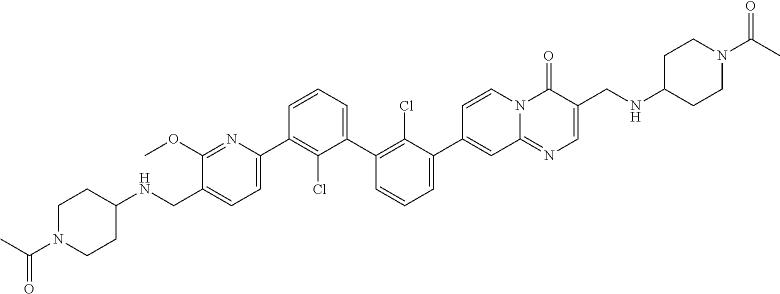 [SMILES: COc1nc(-c2cccc(-c3cccc(-c4ccn5c(=O)c(CNC6CCN(C(C)=O)CC6)cnc5c4)c3Cl)c2Cl)ccc1CNC1CCN(C(C)=O)CC1] | 0.0001 |
| | 3-(((1-acetylpiperidin-4-yl)amino)methyl)-8-(3′-(5-(((1-acetylpiperidin-4-yl)amino)methyl)-6-methoxypyridin-2-yl)-2,2′-dichloro- | |
| | [1,1′-biphenyl]-3-yl)-4H-pyrido[1,2-a]pyrimidin-4-one | |
| 323 | 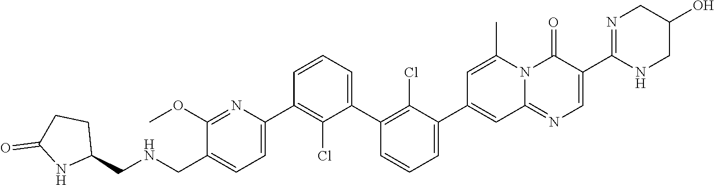 [SMILES: COc1nc(-c2cccc(-c3cccc(-c4cc(C)n5c(=O)c(C6=NCC(O)CN6)cnc5c4)c3Cl)c2Cl)ccc1CNC[C@@H]1CCC(=O)N1] | 0.0059 |
| | 8-(2,2′-dichloro-3′-(6-methoxy-5-(((((S)-5-oxopyrrolidin-2-yl)methyl)amino)methyl)pyridin-2-yl)-[1,1′-biphenyl]-3-yl)- | |
| | 3-(5-hydroxy-1,4,5,6-tetrahydropyrimidin-2-yl)-6-methyl-4H-pyrido[1,2-a]pyrimidin-4-one | |
| 324 | 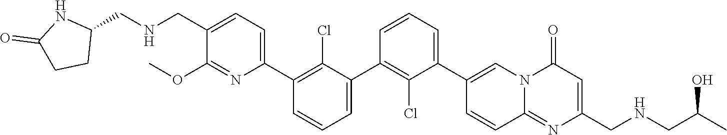 [SMILES: COc1nc(-c2cccc(-c3cccc(-c4ccc5nc(CNC[C@H](C)O)cc(=O)n5c4)c3Cl)c2Cl)ccc1CNC[C@@H]1CCC(=O)N1] | 0.0057 |
| | 7-(2,2′-dichloro-3′-(6-methoxy-5-(((((S)-5-oxopyrrolidin-2-yl)methyl)amino)methyl)pyridin-2-yl)-[1,1′-biphenyl]-3-yl)-2- | |
| | ((((S)-2-hydroxypropyl)amino)methyl)-4H-pyrido[1,2-a]pyrimidin-4-one | |
| 325 | 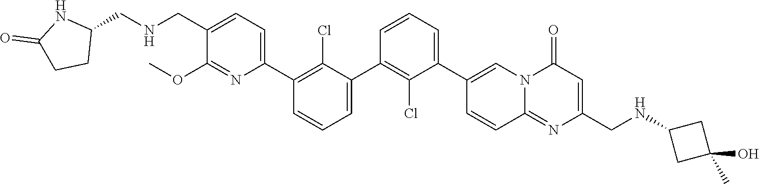 [SMILES: COc1nc(-c2cccc(-c3cccc(-c4ccc5nc(CN[C@H]6C[C@](C)(O)C6)cc(=O)n5c4)c3Cl)c2Cl)ccc1CNC[C@@H]1CCC(=O)N1] | 0.0001 |
| | 7-(2,2′-dichloro-3′-(6-methoxy-5-(((((S)-5-oxopyrrolidin-2-yl)methyl)amino)methyl)pyridin-2-yl)-[1,1′-biphenyl]-3-yl)-2- | |
| | ((((1r,3r)-3-hydroxy-3-methylcyclobutyl)amino)methyl)-4H-pyrido[1,2-a]pyrimidin-4-one | |
| 326 | 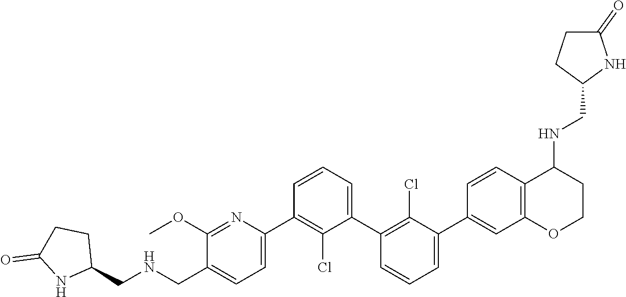 [SMILES: COc1nc(-c2cccc(-c3cccc(-c4ccc5c(c4)OCCC5NC[C@@H]4CCC(=O)N4)c3Cl)c2Cl)ccc1CNC[C@@H]1CCC(=O)N1] | 0.024 |
| | (5S)-5-((((6-(2,2′-dichloro-3′-(4-((((S)-5-oxopyrrolidin-2-yl)methyl)amino)chroman-7-yl)-[1,1′-biphenyl]- | |
| | 3-yl)-2-methoxypyridin-3-yl)methyl)amino)methyl)pyrrolidin-2-one | |
| | mixture of 2 diastereoisomers | |
| 327 | 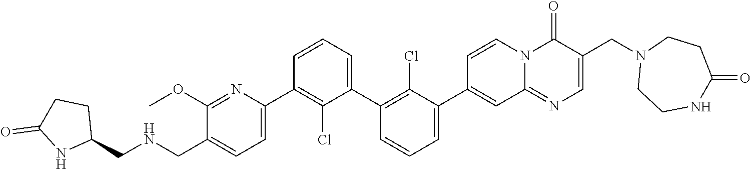 [SMILES: COc1nc(-c2cccc(-c3cccc(-c4ccn5c(=O)c(CN6CCNC(=O)CC6)cnc5c4)c3Cl)c2Cl)ccc1CNC[C@@H]1CCC(=O)N1] | 0.0001 |
| | (S)-8-(2,2′-dichloro-3′-(6-methoxy-5-((((5-oxopyrrolidin-2-yl)methyl)amino)methyl)pyridin-2-yl)-[1,1′-biphenyl]-3-yl)-3-((5- | |
| | oxo-1,4-diazepan-1-yl)methyl)-4H-pyrido[1,2-a]pyrimidin-4-one | |
| 328 | 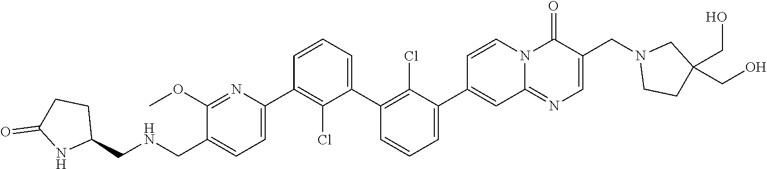 [SMILES: COc1nc(-c2cccc(-c3cccc(-c4ccn5c(=O)c(CN6CCC(CO)(CO)C6)cnc5c4)c3Cl)c2Cl)ccc1CNC[C@@H]1CCC(=O)N1] | 0.0001 |
| | (S)-3-((3,3-bis(hydroxymethyl)pyrrolidin-1-yl)methyl)-8-(2,2′-dichloro-3′-(6-methoxy-5-((((5-oxopyrrolidin-2- | |
| | yl)methyl)amino)methyl)pyridin-2-yl)-[1,1′-biphenyl]-3-yl)-4H-pyrido[1,2-a]pyrimidin-4-one | |
| 329 | 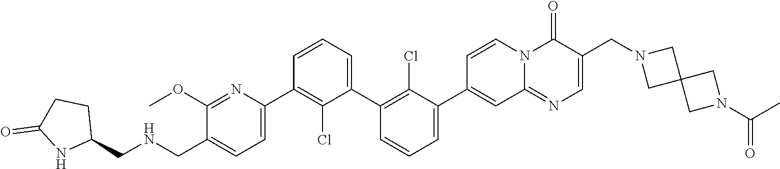 [SMILES: COc1nc(-c2cccc(-c3cccc(-c4ccn5c(=O)c(CN6CC7(C6)CN(C(C)=O)C7)cnc5c4)c3Cl)c2Cl)ccc1CNC[C@@H]1CCC(=O)N1] | 0.0001 |
| | (S)-3-((6-acetyl-2,6-diazaspiro[3.3]heptan-2-yl)methyl)-8-(2,2′-dichloro-3′-(6-methoxy-5-((((5-oxopyrrolidin-2- | |
| | yl)methyl)amino)methyl)pyridin-2-yl)-[1,1′-biphenyl]-3-yl)-4H-pyrido[1,2-a]pyrimidin-4-one | |
| 330 | 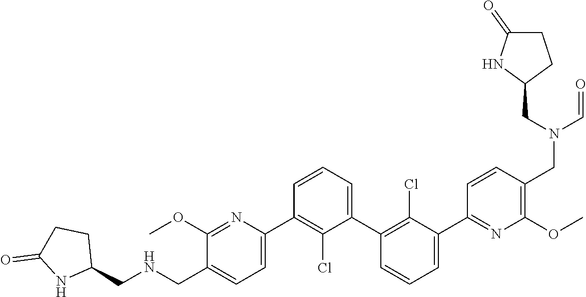 [SMILES: COc1nc(-c2cccc(-c3cccc(-c4ccc(CN(C=O)C[C@@H]5CCC(=O)N5)c(OC)n4)c3Cl)c2Cl)ccc1CNC[C@@H]1CCC(=O)N1] | 0.0008 |
| | N-((6-(2,2′-dichloro-3′-(6-methoxy-5-(((((S)-5-oxopyrrolidin-2-yl)methyl)amino)methyl)pyridin-2- | |
| | yl)-[1,1′-biphenyl]-3-yl)-2-methoxypyridin-3-yl)methyl)-N-(((S)-5-oxopyrrolidin-2- | |
| | yl)methyl)formamide | |
| 331 | 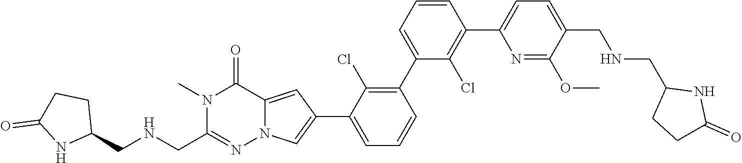 [SMILES: COc1nc(-c2cccc(-c3cccc(-c4cc5c(=O)n(C)c(CNC[C@@H]6CCC(=O)N6)nn5c4)c3Cl)c2Cl)ccc1CNCC1CCC(=O)N1] | 0.31 |
| | 6-(2,2′-dichloro-3′-(6-methoxy-5-((((5-oxopyrrolidin-2-yl)methyl)amino)methyl)pyridin-2-yl)-[1,1′-biphenyl]-3-yl)-3-methyl- | |
| | 2-(((((S)-5-oxopyrrolidin-2-yl)methyl)amino)methyl)pyrrolo[2,1-f][1,2,4]triazin-4(3H)-one | |
| 332 | 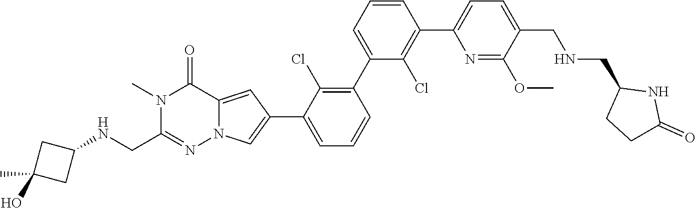 [SMILES: COc1nc(-c2cccc(-c3cccc(-c4cc5c(=O)n(C)c(CN[C@H]6C[C@](C)(O)C6)nn5c4)c3Cl)c2Cl)ccc1CNC[C@@H]1CCC(=O)N1] | 0.50 |
| | 6-(2,2′-dichloro-3′-(6-methoxy-5-(((((S)-5-oxopyrrolidin-2-yl)methyl)amino)methyl)pyridin-2-yl)-[1,1′-biphenyl]-3-yl)- | |
| | 2-((((1r,3r)-3-hydroxy-3-methylcyclobutyl)amino)methyl)-3-methylpyrrolo[2,1-f][1,2,4]triazin-4(3H)-one | |
| 333 | 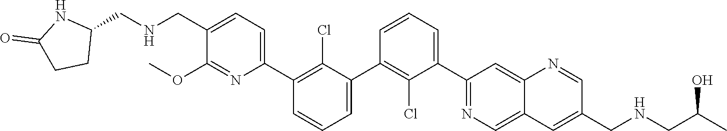 [SMILES: COc1nc(-c2cccc(-c3cccc(-c4cc5ncc(CNC[C@H](C)O)cc5cn4)c3Cl)c2Cl)ccc1CNC[C@@H]1CCC(=O)N1] | 0.0007 |
| | (S)-5-((((6-(2,2′-dichloro-3′-(3-((((S)-2-hydroxypropyl)amino)methyl)-1,6-naphthyridin-7-yl)-[1,1′-biphenyl]-3-yl)-2- | |
| | methoxypyridin-3-yl)methyl)amino)methyl)pyrrolidin-2-one | |
| 334 | 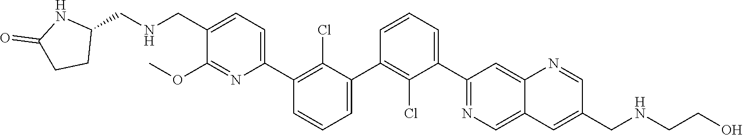 [SMILES: COc1nc(-c2cccc(-c3cccc(-c4cc5ncc(CNCCO)cc5cn4)c3Cl)c2Cl)ccc1CNC[C@@H]1CCC(=O)N1] | 0.0001 |
| | (S)-5-((((6-(2,2′-dichloro-3′-(3-(((2-hydroxyethyl)amino)methyl)-1,6-naphthyridin-7-yl)-[1,1′-biphenyl]-3-yl)-2- | |
| | methoxypyridin-3-yl)methyl)amino)methyl)pyrrolidin-2-one | |
| 335 | 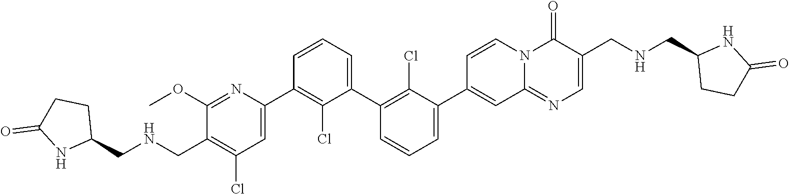 [SMILES: COc1nc(-c2cccc(-c3cccc(-c4ccn5c(=O)c(CNC[C@@H]6CCC(=O)N6)cnc5c4)c3Cl)c2Cl)cc(Cl)c1CNC[C@@H]1CCC(=O)N1] | 0.0021 |
| | 8-(2,2′-dichloro-3′-(4-chloro-6-methoxy-5-(((((S)-5-oxopyrrolidin-2-yl)methyl)amino)methyl)pyridin-2-yl)-[1,1′-biphenyl]-3-yl)-3- | |
| | (((((S)-5-oxopyrrolidin-2-yl)methyl)amino)methyl)-4H-pyrido[1,2-a]pyrimidin-4-one | |
| 336 | 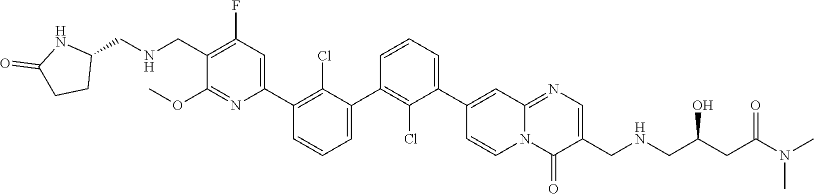 [SMILES: COc1nc(-c2cccc(-c3cccc(-c4ccn5c(=O)c(CNC[C@@H](O)CC(=O)N(C)C)cnc5c4)c3Cl)c2Cl)cc(F)c1CNC[C@@H]1CCC(=O)N1] | 0.0001 |
| | (S)-4-(((8-(2,2′-dichloro-3′-(4-fluoro-6-methoxy-5-(((((S)-5-oxopyrrolidin-2-yl)methyl)amino)methyl)pyridin-2-yl)-[1,1′-biphenyl]-3-yl)- | |
| | 4-oxo-4H-pyrido[1,2-a]pyrimidin-3-yl)methyl)amino)-3-hydroxy-N,N-dimethylbutanamide | |
| 337 | 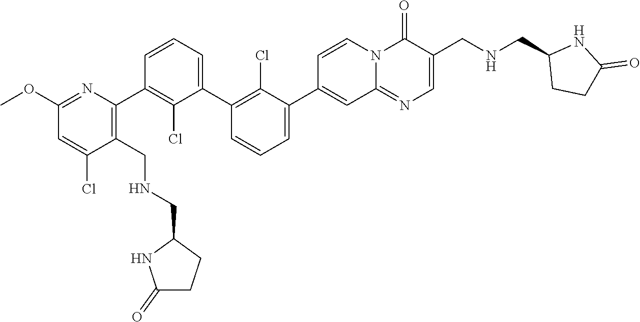 [SMILES: COc1cc(Cl)c(CNC[C@H]2CCC(=O)N2)c(-c2cccc(-c3cccc(-c4ccn5c(=O)c(CNC[C@@H]6CCC(=O)N6)cnc5c4)c3Cl)c2Cl)n1] | 0.047 |
| | 8-(2,2′-dichloro-3′-(4-chloro-6-methoxy-3-(((((R)-5-oxopyrrolidin-2-yl)methyl)amino)methyl)pyridin-2-yl)- | |
| | [1,1′-biphenyl]-3-yl)-3-(((((S)-5-oxopyrrolidin-2-yl)methyl)amino)methyl)-4H-pyrido[1,2-a]pyrimidin-4-one | |
| 338 | 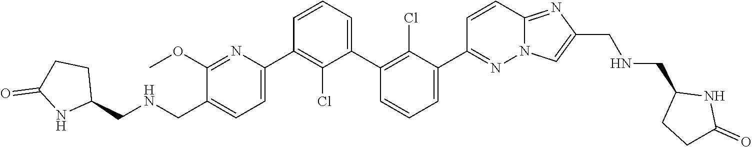 [SMILES: COc1nc(-c2cccc(-c3cccc(-c4ccc5nc(CNC[C@@H]6CCC(=O)N6)cn5n4)c3Cl)c2Cl)ccc1CNC[C@@H]1CCC(=O)N1] | 0.0003 |
| | (S)-5-((((6-(2,2′-dichloro-3′-(2-(((((S)-5-oxopyrrolidin-2-yl)methyl)amino)methyl)imidazo[1,2-b]pyridazin-6-yl)-[1,1′- | |
| | biphenyl]-3-yl)-2-methoxypyridin-3-yl)methyl)amino)methyl)pyrrolidin-2-one | |
| 339 | 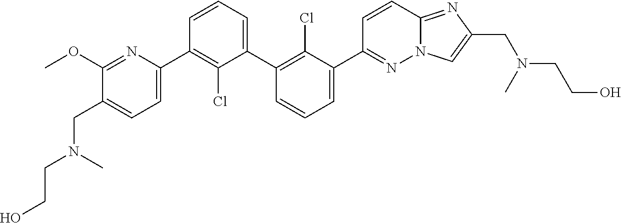 [SMILES: COc1nc(-c2cccc(-c3cccc(-c4ccc5nc(CN(C)CCO)cn5n4)c3Cl)c2Cl)ccc1CN(C)CCO] | 0.026 |
| | 2-(((6-(2,2′-dichloro-3′-(2-(((2-hydroxyethyl)(methyl)amino)methyl)imidazo[1,2-b]pyridazin-6-yl)-[1,1′- | |
| | biphenyl]-3-yl)-2-methoxypyridin-3-yl)methyl)(methyl)amino)ethan-1-ol | |
| 340 | 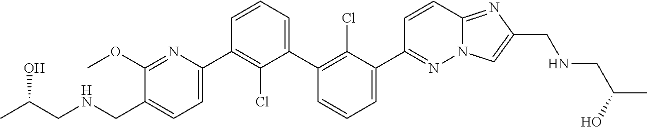 [SMILES: COc1nc(-c2cccc(-c3cccc(-c4ccc5nc(CNC[C@H](C)O)cn5n4)c3Cl)c2Cl)ccc1CNC[C@H](C)O] | 0.0035 |
| | (S)-1-(((6-(2,2′-dichloro-3′-(2-((((S)-2-hydroxypropyl)amino)methyl)imidazo[1,2-b]pyridazin-6-yl)-[1,1′- | |
| | biphenyl]-3-yl)-2-methoxypyridin-3-yl)methyl)amino)propan-2-ol | |
| 341 | 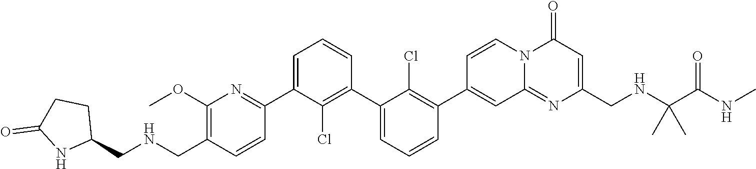 [SMILES: CNC(=O)C(C)(C)NCc1cc(=O)n2ccc(-c3cccc(-c4cccc(-c5ccc(CNC[C@@H]6CCC(=O)N6)c(OC)n5)c4Cl)c3Cl)cc2n1] | 0.0001 |
| | (S)-2-(((8-(2,2′-dichloro-3′-(6-methoxy-5-((((5-oxopyrrolidin-2-yl)methyl)amino)methyl)pyridin-2-yl)-[1,1′-biphenyl]-3-yl)-4- | |
| | oxo-4H-pyrido[1,2-a]pyrimidin-2-yl)methyl)amino)-N,2-dimethylpropanamide | |
| 342 | 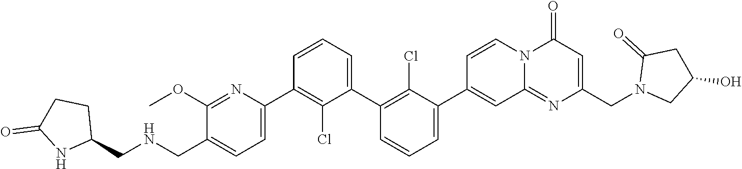 [SMILES: COc1nc(-c2cccc(-c3cccc(-c4ccn5c(=O)cc(CN6C[C@@H](O)CC6=O)nc5c4)c3Cl)c2Cl)ccc1CNC[C@@H]1CCC(=O)N1] | 0.0001 |
| | 8-(2,2′-dichloro-3′-(6-methoxy-5-(((((S)-5-oxopyrrolidin-2-yl)methyl)amino)methyl)pyridin-2-yl)-[1,1′-biphenyl]-3-yl)-2-(((S)- | |
| | 4-hydroxy-2-oxopyrrolidin-1-yl)methyl)-4H-pyrido[1,2-a]pyrimidin-4-one | |
| 343 | 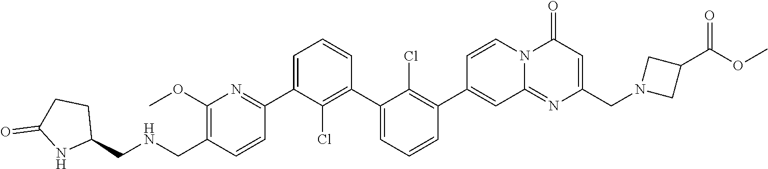 [SMILES: COC(=O)C1CN(Cc2cc(=O)n3ccc(-c4cccc(-c5cccc(-c6ccc(CNC[C@@H]7CCC(=O)N7)c(OC)n6)c5Cl)c4Cl)cc3n2)C1] | 0.0001 |
| | methyl (S)-1-((8-(2,2′-dichloro-3′-(6-methoxy-5-((((5-oxopyrrolidin-2-yl)methyl)amino)methyl)pyridin-2-yl)-[1,1′-biphenyl]-3-yl)- | |
| | 4-oxo-4H-pyrido[1,2-a]pyrimidin-2-yl)methyl)azetidine-3-carboxylate | |
| 344 | 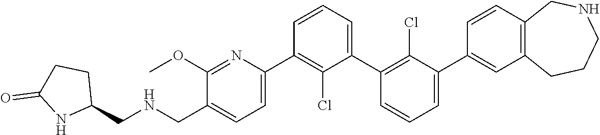 [SMILES: COc1nc(-c2cccc(-c3cccc(-c4ccc5c(c4)CCCNC5)c3Cl)c2Cl)ccc1CNC[C@@H]1CCC(=O)N1] | 0.57 |
| | (S)-5-((((6-(2,2′-dichloro-3′-(2,3,4,5-tetrahydro-1H-benzo[c]azepin-7-yl)-[1,1′-biphenyl]-3-yl)-2- | |
| | methoxypyridin-3-yl)methyl)amino)methyl)pyrrolidin-2-one | |
| 345 | 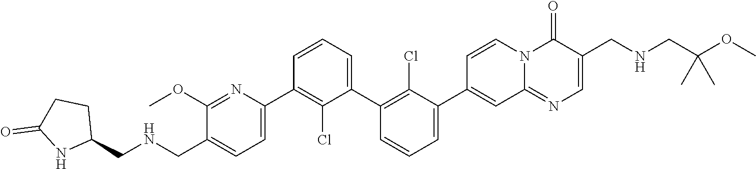 [SMILES: COc1nc(-c2cccc(-c3cccc(-c4ccn5c(=O)c(CNCC(C)(C)OC)cnc5c4)c3Cl)c2Cl)ccc1CNC[C@@H]1CCC(=O)N1] | 0.042 |
| | (S)-8-(2,2′-dichloro-3′-(6-methoxy-5-((((5-oxopyrrolidin-2-yl)methyl)amino)methyl)pyridin-2-yl)-[1,1′-biphenyl]-3-yl)-3- | |
| | (((2-methoxy-2-methylpropyl)amino)methyl)-4H-pyrido[1,2-a]pyrimidin-4-one | |
| 346 | 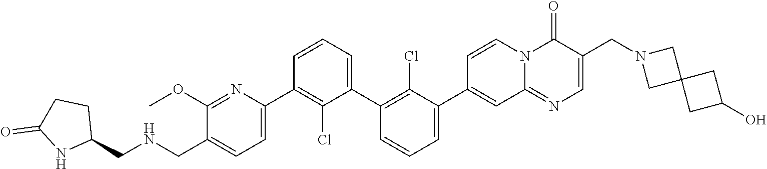 [SMILES: COc1nc(-c2cccc(-c3cccc(-c4ccn5c(=O)c(CN6CC7(CC(O)C7)C6)cnc5c4)c3Cl)c2Cl)ccc1CNC[C@@H]1CCC(=O)N1] | 1.3 |
| | (S)-8-(2,2′-dichloro-3′-(6-methoxy-5-((((5-oxopyrrolidin-2-yl)methyl)amino)methyl)pyridin-2-yl)-[1,1′-biphenyl]-3-yl)-3-((6- | |
| | hydroxy-2-azaspiro[3.3]heptan-2-yl)methyl)-4H-pyrido[1,2-a]pyrimidin-4-one | |
| 347 | 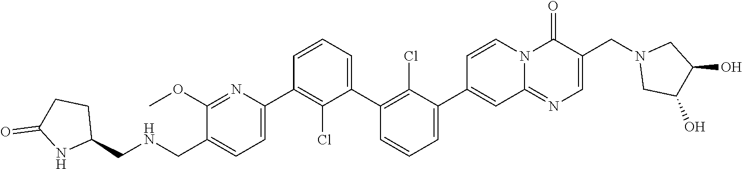 [SMILES: COc1nc(-c2cccc(-c3cccc(-c4ccn5c(=O)c(CN6C[C@@H](O)[C@H](O)C6)cnc5c4)c3Cl)c2Cl)ccc1CNC[C@@H]1CCC(=O)N1] | 0.53 |
| | 8-(2,2′-dichloro-3′-(6-methoxy-5-(((((S)-5-oxopyrrolidin-2-yl)methyl)amino)methyl)pyridin-2-yl)-[1,1′-biphenyl]-3-yl)-3- | |
| | (((3R,4R)-3,4-dihydroxypyrrolidin-1-yl)methyl)-4H-pyrido[1,2-a]pyrimidin-4-one | |
| 348 | 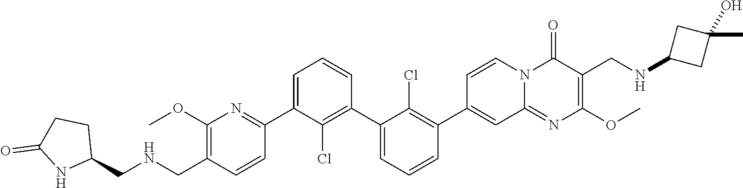 | 0.0022 |
| | (S)-8-(2,2′-dichloro-3′-(6-methoxy-5-((((5-oxopyrrolidin-2-yl)methyl)amino)methyl)pyridin-2-yl)-[1,1′-biphenyl]-3-yl)-3-(((3- | |
| | hydroxy-3-methylcyclobutyl)amino)methyl)-2-methoxy-4H-pyrido[1,2-a]pyrimidin-4-one | |
| 349 | 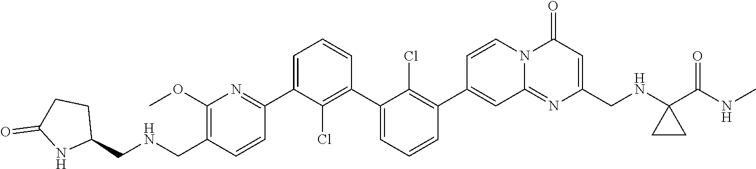 [SMILES: CNC(=O)C1(NCc2cc(=O)n3ccc(-c4cccc(-c5cccc(-c6ccc(CNC[C@@H]7CCC(=O)N7)c(OC)n6)c5Cl)c4Cl)cc3n2)CC1] | 0.0002 |
| | (S)-1-(((8-(2,2′-dichloro-3′-(6-methoxy-5-((((5-oxopyrrolidin-2-yl)methyl)amino)methyl)pyridin-2-yl)-[1,1′-biphenyl]-3-yl)-4- | |
| | oxo-4H-pyrido[1,2-a]pyrimidin-2-yl)methyl)amino)-N-methylcyclopropane-1-carboxamide | |
| 350 | 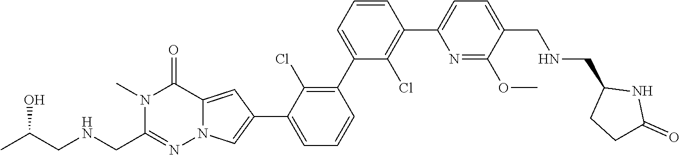 [SMILES: COc1nc(-c2cccc(-c3cccc(-c4cc5c(=O)n(C)c(CNC[C@H](C)O)nn5c4)c3Cl)c2Cl)ccc1CNC[C@@H]1CCC(=O)N1] | 0.0001 |
| | 6-(2,2′-dichloro-3′-(6-methoxy-5-(((((S)-5-oxopyrrolidin-2-yl)methyl)amino)methyl)pyridin-2-yl)-[1,1′-biphenyl]-3- | |
| | yl)-2-((((S)-2-hydroxypropyl)amino)methyl)-3-methylpyrrolo[2,1-f][1,2,4]triazin-4(3H)-one | |
| 351 | 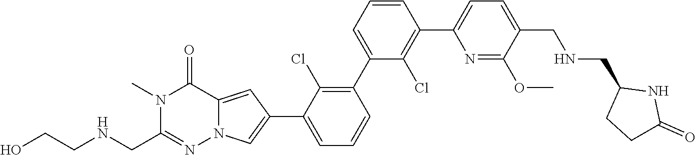 [SMILES: COc1nc(-c2cccc(-c3cccc(-c4cc5c(=O)n(C)c(CNCCO)nn5c4)c3Cl)c2Cl)ccc1CNC[C@@H]1CCC(=O)N1] | 0.20 |
| | (S)-6-(2,2′-dichloro-3′-(6-methoxy-5-((((5-oxopyrrolidin-2-yl)methyl)amino)methyl)pyridin-2-yl)-[1,1′-biphenyl]-3-yl)- | |
| | 2-(((2-hydroxyethyl)amino)methyl)-3-methylpyrrolo[2,1-f][1,2,4]triazin-4(3H)-one | |
| 352 | 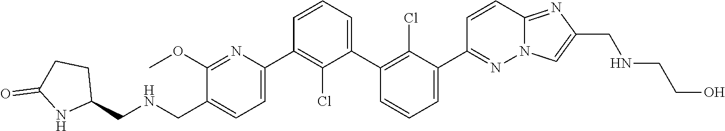 [SMILES: COc1nc(-c2cccc(-c3cccc(-c4ccc5nc(CNCCO)cn5n4)c3Cl)c2Cl)ccc1CNC[C@@H]1CCC(=O)N1] | 0.0005 |
| | (S)-5-((((6-(2,2′-dichloro-3′-(2-(((2-hydroxyethyl)amino)methyl)imidazo[1,2-b]pyridazin-6-yl)-[1,1′-biphenyl]-3-yl)- | |
| | 2-methoxypyridin-3-yl)methyl)amino)methyl)pyrrolidin-2-one | |
| 353 | 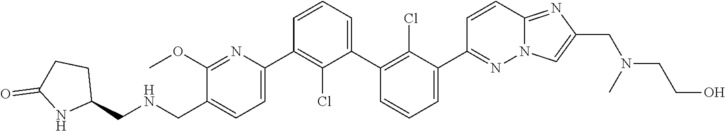 [SMILES: COc1nc(-c2cccc(-c3cccc(-c4ccc5nc(CN(C)CCO)cn5n4)c3Cl)c2Cl)ccc1CNC[C@@H]1CCC(=O)N1] | 0.0000 |
| | (S)-5-((((6-(2,2′-dichloro-3′-(2-(((2-hydroxyethyl)(methyl)amino)methyl)imidazo[1,2-b]pyridazin-6-yl)-[1,1′-biphenyl]- | |
| | 3-yl)-2-methoxypyridin-3-yl)methyl)amino)methyl)pyrrolidin-2-one | |
| 354 | 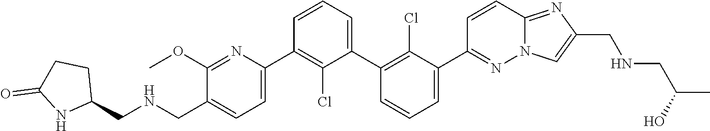 [SMILES: COc1nc(-c2cccc(-c3cccc(-c4ccc5nc(CNC[C@H](C)O)cn5n4)c3Cl)c2Cl)ccc1CNC[C@@H]1CCC(=O)N1] | 0.0008 |
| | (S)-5-((((6-(2,2′-dichloro-3′-(2-((((S)-2-hydroxypropyl)amino)methyl)imidazo[1,2-b]pyridazin-6-yl)-[1,1′-biphenyl]-3- | |
| | yl)-2-methoxypyridin-3-yl)methyl)amino)methyl)pyrrolidin-2-one | |
| 355 | 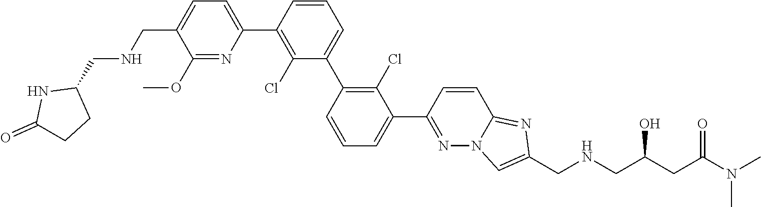 [SMILES: COc1nc(-c2cccc(-c3cccc(-c4ccc5nc(CNC[C@@H](O)CC(=O)N(C)C)cn5n4)c3Cl)c2Cl)ccc1CNC[C@@H]1CCC(=O)N1] | 0.0001 |
| | (S)-4-(((6-(2,2′-dichloro-3′-(6-methoxy-5-(((((S)-5-oxopyrrolidin-2-yl)methyl)amino)methyl)pyridin-2-yl)-[1,1′-biphenyl]-3- | |
| | yl)imidazo[1,2-b]pyridazin-2-yl)methyl)amino)-3-hydroxy-N,N-dimethylbutanamide | |
| 356 | 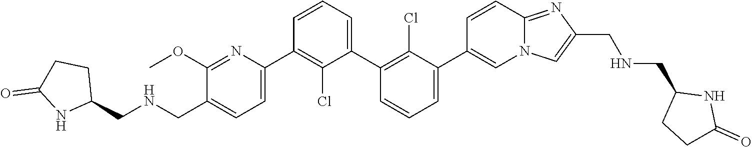 [SMILES: COc1nc(-c2cccc(-c3cccc(-c4ccc5nc(CNC[C@@H]6CCC(=O)N6)cn5c4)c3Cl)c2Cl)ccc1CNC[C@@H]1CCC(=O)N1] | 0.086 |
| | (S)-5-((((6-(2,2′-dichloro-3′-(2-(((((S)-5-oxopyrrolidin-2-yl)methyl)amino)methyl)imidazo[1,2-a]pyridin-6-yl)-[1,1′-biphenyl]- | |
| | 3-yl)-2-methoxypyridin-3-yl)methyl)amino)methyl)pyrrolidin-2-one | |
| 357 | 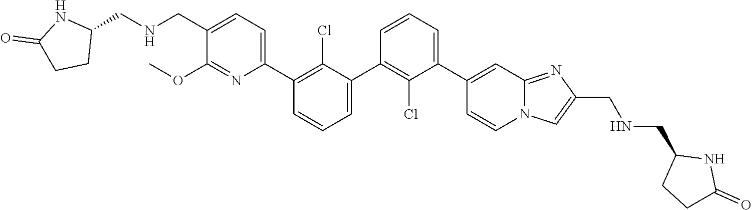 [SMILES: COc1nc(-c2cccc(-c3cccc(-c4ccn5cc(CNC[C@@H]6CCC(=O)N6)nc5c4)c3Cl)c2Cl)ccc1CNC[C@@H]1CCC(=O)N1] | 0.050 |
| | (S)-5-((((6-(2,2′-dichloro-3′-(2-(((((S)-5-oxopyrrolidin-2-yl)methyl)amino)methyl)imidazo[1,2-a]pyridin-7-yl)-[1,1′- | |
| | biphenyl]-3-yl)-2-methoxypyridin-3-yl)methyl)amino)methyl)pyrrolidin-2-one | |
| 358 | 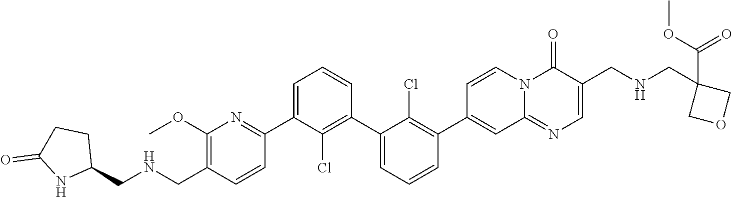 [SMILES: COC(=O)C1(CNCc2cnc3cc(-c4cccc(-c5cccc(-c6ccc(CNC[C@@H]7CCC(=O)N7)c(OC)n6)c5Cl)c4Cl)ccn3c2=O)COC1] | 0.0001 |
| | methyl (S)-3-((((8-(2,2′-dichloro-3′-(6-methoxy-5-((((5-oxopyrrolidin-2-yl)methyl)amino)methyl)pyridin-2-yl)-[1,1′- | |
| | biphenyl]-3-yl)-4-oxo-4H-pyrido[1,2-a]pyrimidin-3-yl)methyl)amino)methyl)oxetane-3-carboxylate | |
| 359 | 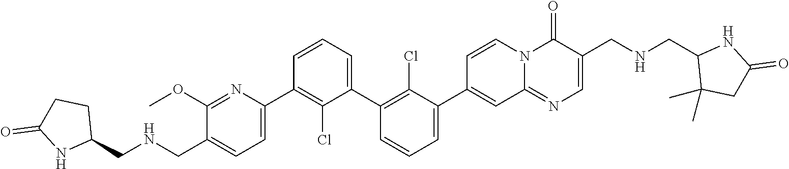 [SMILES: COc1nc(-c2cccc(-c3cccc(-c4ccn5c(=O)c(CNCC6NC(=O)CC6(C)C)cnc5c4)c3Cl)c2Cl)ccc1CNC[C@@H]1CCC(=O)N1] | 0.067 |
| | 8-(2,2′-dichloro-3′-(6-methoxy-5-(((((S)-5-oxopyrrolidin-2-yl)methyl)amino)methyl)pyridin-2-yl)-[1,1′-biphenyl]-3-yl)-3-((((3,3- | |
| | dimethyl-5-oxopyrrolidin-2-yl)methyl)amino)methyl)-4H-pyrido[1,2-a]pyrimidin-4-one | |
| | mixture of 2 diastereoisomers | |
| 360 | 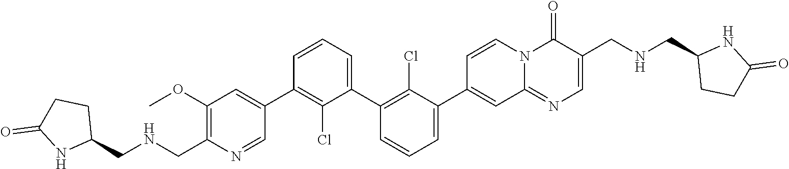 [SMILES: COc1cc(-c2cccc(-c3cccc(-c4ccn5c(=O)c(CNC[C@@H]6CCC(=O)N6)cnc5c4)c3Cl)c2Cl)cnc1CNC[C@@H]1CCC(=O)N1] | 0.0007 |
| | 8-(2,2′-dichloro-3′-(5-methoxy-6-(((((S)-5-oxopyrrolidin-2-yl)methyl)amino)methyl)pyridin-3-yl)-[1,1′-biphenyl]-3-yl)-3-(((((S)-5- | |
| | oxopyrrolidin-2-yl)methyl)amino)methyl)-4H-pyrido[1,2-a]pyrimidin-4-one | |
| 361 | 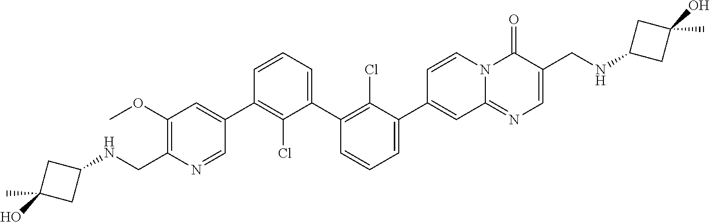 | 0.039 |
| | 8-(2,2′-dichloro-3′-(6-((((1r,3r)-3-hydroxy-3-methylcyclobutyl)amino)methyl)-5-methoxypyridin-3-yl)-[1,1′-biphenyl]-3- | |
| | yl)-3-((((1r,3r)-3-hydroxy-3-methylcyclobutyl)amino)methyl)-4H-pyrido[1,2-a]pyrimidin-4-one | |
| 362 | 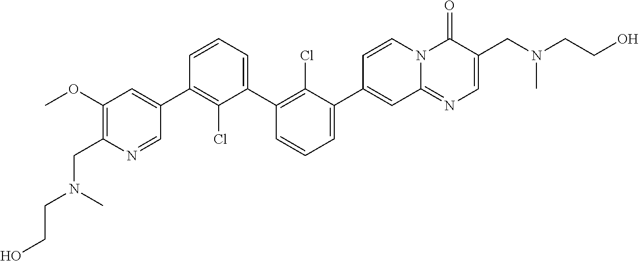 [SMILES: COc1cc(-c2cccc(-c3cccc(-c4ccn5c(=O)c(CN(C)CCO)cnc5c4)c3Cl)c2Cl)cnc1CN(C)CCO] | 0.036 |
| | 8-(2,2′-dichloro-3′-(6-(((2-hydroxyethyl)(methyl)amino)methyl)-5-methoxypyridin-3-yl)-[1,1′-biphenyl]-3- | |
| | yl)-3-(((2-hydroxyethyl)(methyl)amino)methyl)-4H-pyrido[1,2-a]pyrimidin-4-one | |
| 363 | 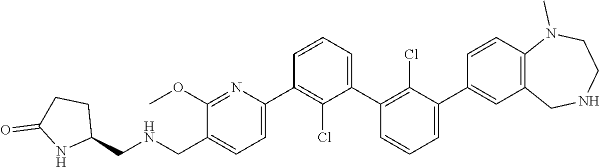 [SMILES: COc1nc(-c2cccc(-c3cccc(-c4ccc5c(c4)CNCCN5C)c3Cl)c2Cl)ccc1CNC[C@@H]1CCC(=O)N1] | 0.085 |
| | (S)-5-((((6-(2,2′-dichloro-3′-(1-methyl-2,3,4,5-tetrahydro-1H-benzo[e][1,4]diazepin-7-yl)-[1,1′- | |
| | biphenyl]-3-yl)-2-methoxypyridin-3-yl)methyl)amino)methyl)pyrrolidin-2-one | |
| 364 | 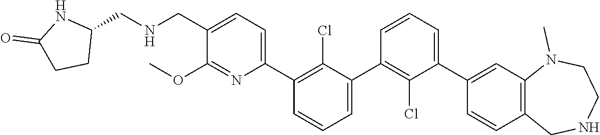 [SMILES: COc1nc(-c2cccc(-c3cccc(-c4ccc5c(c4)N(C)CCNC5)c3Cl)c2Cl)ccc1CNC[C@@H]1CCC(=O)N1] | 0.15 |
| | (S)-5-((((6-(2,2′-dichloro-3′-(1-methyl-2,3,4,5-tetrahydro-1H-benzo[e][1,4]diazepin-8-yl)-[1,1′- | |
| | biphenyl]-3-yl)-2-methoxypyridin-3-yl)methyl)amino)methyl)pyrrolidin-2-one | |
| 365 | 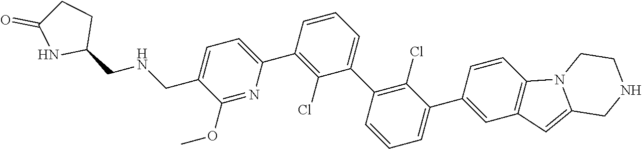 [SMILES: COc1nc(-c2cccc(-c3cccc(-c4ccc5c(c4)cc4n5CCNC4)c3Cl)c2Cl)ccc1CNC[C@@H]1CCC(=O)N1] | 0.013 |
| | (S)-5-((((6-(2,2′-dichloro-3′-(1,2,3,4-tetrahydropyrazino[1,2-a]indol-8-yl)-[1,1′-biphenyl]-3-yl)-2- | |
| | methoxypyridin-3-yl)methyl)amino)methyl)pyrrolidin-2-one | |
| 366 | 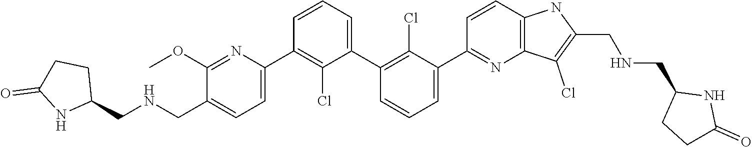 [SMILES: COc1nc(-c2cccc(-c3cccc(-c4ccc5[nH]c(CNC[C@@H]6CCC(=O)N6)c(Cl)c5n4)c3Cl)c2Cl)ccc1CNC[C@@H]1CCC(=O)N1] | 0.0015 |
| | (S)-5-((((3-chloro-5-(2,2′-dichloro-3′-(6-methoxy-5-(((((S)-5-oxopyrrolidin-2-yl)methyl)amino)methyl)pyridin-2-yl)-[1,1′- | |
| | biphenyl]-3-yl)-1-methyl-1H-pyrrolo[3,2-b]pyridin-2-yl)methyl)amino)methyl)pyrrolidin-2-one | |
| 367 | 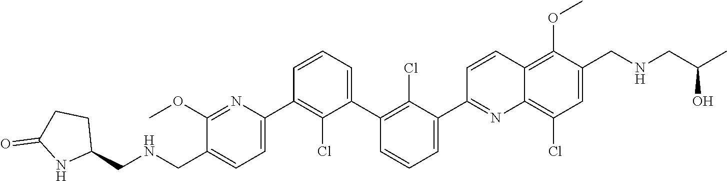 [SMILES: COc1nc(-c2cccc(-c3cccc(-c4ccc5c(OC)c(CNC[C@@H](C)O)cc(Cl)c5n4)c3Cl)c2Cl)ccc1CNC[C@@H]1CCC(=O)N1] | 0.021 |
| | (S)-5-((((6-(2,2′-dichloro-3′-(8-chloro-6-((((R)-2-hydroxypropyl)amino)methyl)-5-methoxyquinolin-2-yl)-[1,1′-biphenyl]-3- | |
| | yl)-2-methoxypyridin-3-yl)methyl)amino)methyl)pyrrolidin-2-one | |
| 368 | 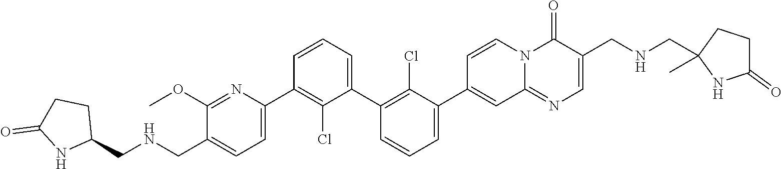 [SMILES: COc1nc(-c2cccc(-c3cccc(-c4ccn5c(=O)c(CNCC6(C)CCC(=O)N6)cnc5c4)c3Cl)c2Cl)ccc1CNC[C@@H]1CCC(=O)N1] | 0.0010 |
| | 8-(2,2′-dichloro-3′-(6-methoxy-5-(((((S)-5-oxopyrrolidin-2-yl)methyl)amino)methyl)pyridin-2-yl)-[1,1′-biphenyl]-3-yl)-3-((((2- | |
| | methyl-5-oxopyrrolidin-2-yl)methyl)amino)methyl)-4H-pyrido[1,2-a]pyrimidin-4-one | |
| | mixture of 2 diastereoisomers | |
| 369 | 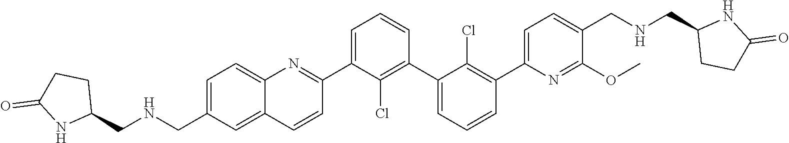 [SMILES: COc1nc(-c2cccc(-c3cccc(-c4ccc5cc(CNC[C@@H]6CCC(=O)N6)ccc5n4)c3Cl)c2Cl)ccc1CNC[C@@H]1CCC(=O)N1] | 0.56 |
| | (S)-5-((((6-(2,2′-dichloro-3′-(6-(((((S)-5-oxopyrrolidin-2-yl)methyl)amino)methyl)quinolin-2-yl)-[1,1′-biphenyl]-3-yl)-2- | |
| | methoxypyridin-3-yl)methyl)amino)methyl)pyrrolidin-2-one | |
| 370 | 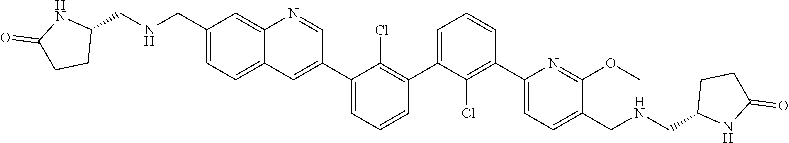 [SMILES: COc1nc(-c2cccc(-c3cccc(-c4cnc5cc(CNC[C@@H]6CCC(=O)N6)ccc5c4)c3Cl)c2Cl)ccc1CNC[C@@H]1CCC(=O)N1] | 0.58 |
| | (S)-5-((((3-(2,2′-dichloro-3′-(6-methoxy-5-(((((S)-5-oxopyrrolidin-2-yl)methyl)amino)methyl)pyridin-2-yl)-[1,1′-biphenyl]-3- | |
| | yl)quinolin-7-yl)methyl)amino)methyl)pyrrolidin-2-one | |
| 371 | 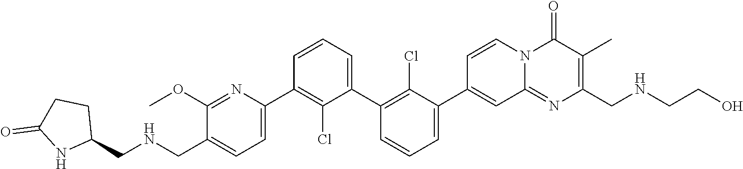 [SMILES: COc1nc(-c2cccc(-c3cccc(-c4ccn5c(=O)c(C)c(CNCCO)nc5c4)c3Cl)c2Cl)ccc1CNC[C@@H]1CCC(=O)N1] | 0.033 |
| | (S)-8-(2,2′-dichloro-3′-(6-methoxy-5-((((5-oxopyrrolidin-2-yl)methyl)amino)methyl)pyridin-2-yl)-[1,1′-biphenyl]-3-yl)-2-(((2- | |
| | hydroxyethyl)amino)methyl)-3-methyl-4H-pyrido[1,2-a]pyrimidin-4-one | |
| 372 | 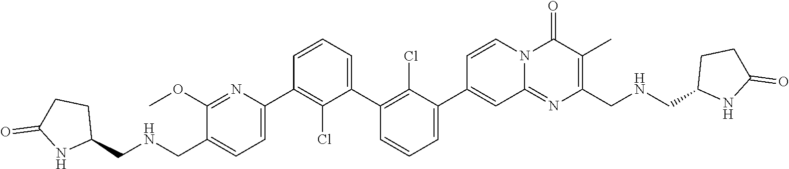 [SMILES: COc1nc(-c2cccc(-c3cccc(-c4ccn5c(=O)c(C)c(CNC[C@@H]6CCC(=O)N6)nc5c4)c3Cl)c2Cl)ccc1CNC[C@@H]1CCC(=O)N1] | 0.060 |
| | 8-(2,2′-dichloro-3′-(6-methoxy-5-(((((S)-5-oxopyrrolidin-2-yl)methyl)amino)methyl)pyridin-2-yl)-[1,1′-biphenyl]-3-yl)-3-methyl-2- | |
| | (((((S)-5-oxopyrrolidin-2-yl)methyl)amino)methyl)-4H-pyrido[1,2-a]pyrimidin-4-one | |
| 373 | 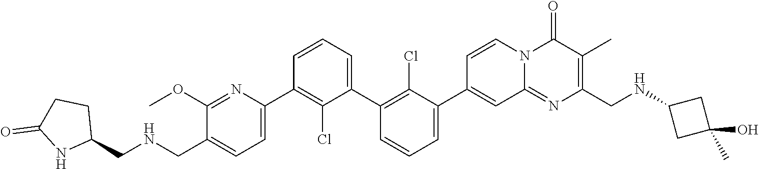 [SMILES: COc1nc(-c2cccc(-c3cccc(-c4ccn5c(=O)c(C)c(CN[C@H]6C[C@](C)(O)C6)nc5c4)c3Cl)c2Cl)ccc1CNC[C@@H]1CCC(=O)N1] | 0.033 |
| | 8-(2,2′-dichloro-3′-(6-methoxy-5-(((((S)-5-oxopyrrolidin-2-yl)methyl)amino)methyl)pyridin-2-yl)-[1,1′-biphenyl]-3-yl)-2- | |
| | ((((1r,3r)-3-hydroxy-3-methylcyclobutyl)amino)methyl)-3-methyl-4H-pyrido[1,2-a]pyrimidin-4-one | |
| 374 | 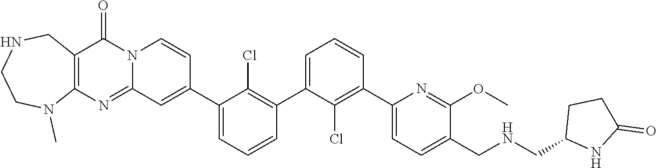 [SMILES: COc1nc(-c2cccc(-c3cccc(-c4ccn5c(=O)c6c(nc5c4)N(C)CCNC6)c3Cl)c2Cl)ccc1CNC[C@@H]1CCC(=O)N1] | 0.0065 |
| | (S)-10-(2,2′-dichloro-3′-(6-methoxy-5-((((5-oxopyrrolidin-2-yl)methyl)amino)methyl)pyridin-2-yl)-[1,1′- | |
| | biphenyl]-3-yl)-1-methyl-2,3,4,5-tetrahydropyrido[1′,2′:1,2]pyrimido[4,5-e][1,4]diazepin-6(1H)-one | |
| 375 | 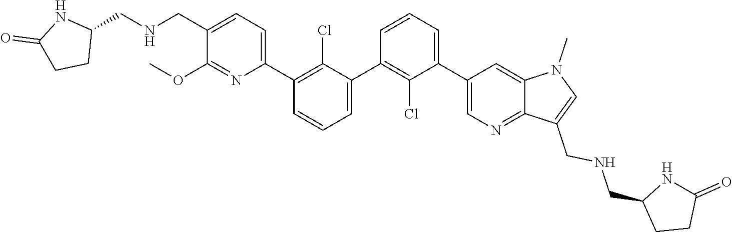 [SMILES: COc1nc(-c2cccc(-c3cccc(-c4cnc5c(CNC[C@@H]6CCC(=O)N6)cn(C)c5c4)c3Cl)c2Cl)ccc1CNC[C@@H]1CCC(=O)N1] | 0.18 |
| | (S)-5-((((6-(2,2′-dichloro-3′-(1-methyl-3-(((((S)-5-oxopyrrolidin-2-yl)methyl)amino)methyl)-1H-pyrrolo[3,2-b]pyridin-6-yl)- | |
| | [1,1′-biphenyl]-3-yl)-2-methoxypyridin-3-yl)methyl)amino)methyl)pyrrolidin-2-one | |
| 376 | 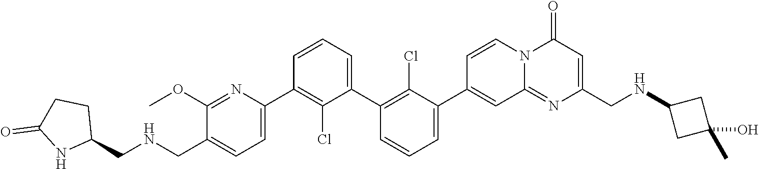 | 0.0007 |
| | (S)-8-(2,2′-dichloro-3′-(6-methoxy-5-((((5-oxopyrrolidin-2-yl)methyl)amino)methyl)pyridin-2-yl)-[1,1′-biphenyl]-3-yl)-2-(((3- | |
| | hydroxy-3-methylcyclobutyl)amino)methyl)-4H-pyrido[1,2-a]pyrimidin-4-one | |
| 377 | 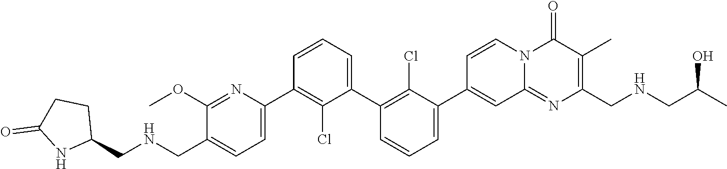 [SMILES: COc1nc(-c2cccc(-c3cccc(-c4ccn5c(=O)c(C)c(CNC[C@H](C)O)nc5c4)c3Cl)c2Cl)ccc1CNC[C@@H]1CCC(=O)N1] | 0.021 |
| | 8-(2,2′-dichloro-3′-(6-methoxy-5-(((((S)-5-oxopyrrolidin-2-yl)methyl)amino)methyl)pyridin-2-yl)-[1,1′-biphenyl]-3-yl)-2- | |
| | ((((S)-2-hydroxypropyl)amino)methyl)-3-methyl-4H-pyrido[1,2-a]pyrimidin-4-one | |
| 378 | 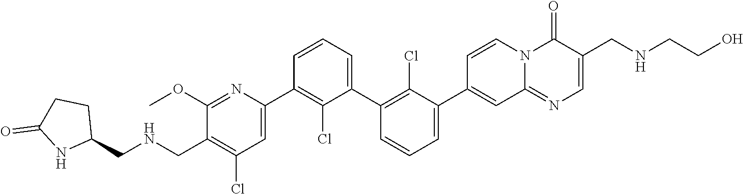 [SMILES: COc1nc(-c2cccc(-c3cccc(-c4ccn5c(=O)c(CNCCO)cnc5c4)c3Cl)c2Cl)cc(Cl)c1CNC[C@@H]1CCC(=O)N1] | 0.11 |
| | (S)-8-(2,2′-dichloro-3′-(4-chloro-6-methoxy-5-((((5-oxopyrrolidin-2-yl)methyl)amino)methyl)pyridin-2-yl)-[1,1′-biphenyl]-3- | |
| | yl)-3-(((2-hydroxyethyl)amino)methyl)-4H-pyrido[1′,2-a]pyrimidin-4-one | |
| 379 | 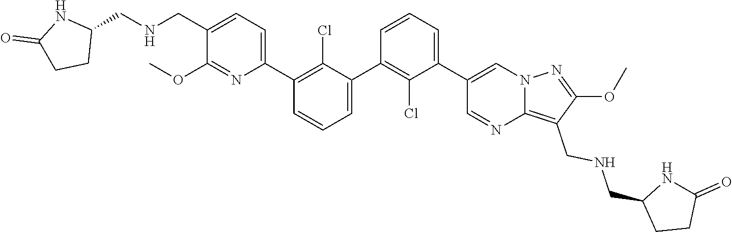 [SMILES: COc1nc(-c2cccc(-c3cccc(-c4cnc5c(CNC[C@@H]6CCC(=O)N6)c(OC)nn5c4)c3Cl)c2Cl)ccc1CNC[C@@H]1CCC(=O)N1] | 0.0056 |
| | (S)-5-((((6-(2,2′-dichloro-3′-(2-methoxy-3-(((((S)-5-oxopyrrolidin-2-yl)methyl)amino)methyl)pyrazolo[1,5-a]pyrimidin-6-yl)- | |
| | [1,1′-biphenyl]-3-yl)-2-methoxypyridin-3-yl)methyl)amino)methyl)pyrrolidin-2-one | |
| 380 | 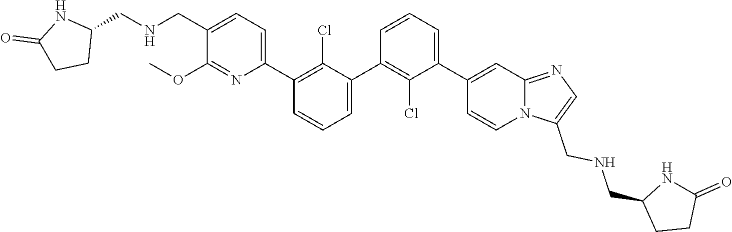 [SMILES: COc1nc(-c2cccc(-c3cccc(-c4ccn5c(CNC[C@@H]6CCC(=O)N6)cnc5c4)c3Cl)c2Cl)ccc1CNC[C@@H]1CCC(=O)N1] | 0.024 |
| | (S)-5-((((6-(2,2′-dichloro-3′-(3-(((((S)-5-oxopyrrolidin-2-yl)methyl)amino)methyl)imidazo[1,2-a]pyridin-7-yl)-[1,1′- | |
| | biphenyl]-3-yl)-2-methoxypyridin-3-yl)methyl)amino)methyl)pyrrolidin-2-one | |
| 381 | 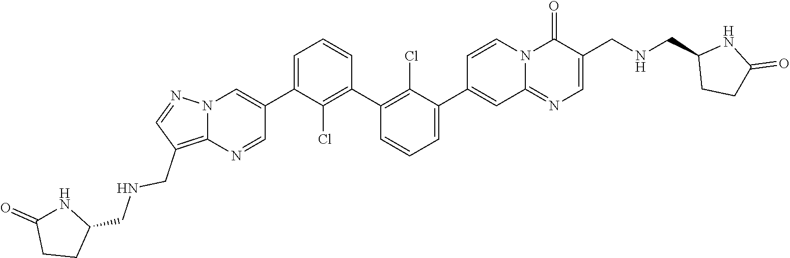 [SMILES: O=C1CC[C@@H](CNCc2cnc3cc(-c4cccc(-c5cccc(-c6cnc7c(CNC[C@@H]8CCC(=O)N8)cnn7c6)c5Cl)c4Cl)ccn3c2=O)N1] | 0.0059 |
| | 8-(2,2′-dichloro-3′-(3-(((((S)-5-oxopyrrolidin-2-yl)methyl)amino)methyl)pyrazolo[1,5-a]pyrimidin-6-yl)-[1,1′-biphenyl]-3-yl)-3- | |
| | (((((S)-5-oxopyrrolidin-2-yl)methyl)amino)methyl)-4H-pyrido[1,2-a]pyrimidin-4-one | |
| 382 | 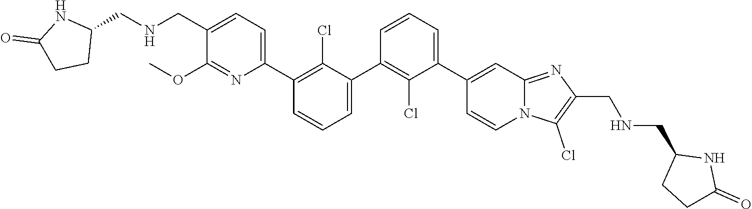 [SMILES: COc1nc(-c2cccc(-c3cccc(-c4ccn5c(Cl)c(CNC[C@@H]6CCC(=O)N6)nc5c4)c3Cl)c2Cl)ccc1CNC[C@@H]1CCC(=O)N1] | 0.11 |
| | (S)-5-((((3-chloro-7-(2,2′-dichloro-3′-(6-methoxy-5-(((((S)-5-oxopyrrolidin-2-yl)methyl)amino)methyl)pyridin-2-yl)-[1,1′- | |
| | biphenyl]-3-yl)imidazo[1,2-a]pyridin-2-yl)methyl)amino)methyl)pyrrolidin-2-one | |
| 383 | 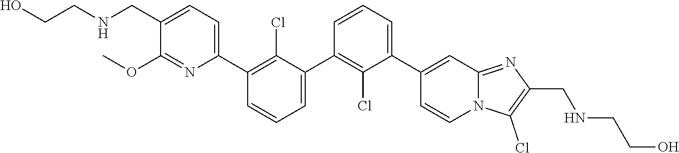 [SMILES: COc1nc(-c2cccc(-c3cccc(-c4ccn5c(Cl)c(CNCCO)nc5c4)c3Cl)c2Cl)ccc1CNCCO] | 0.040 |
| | 2-(((3-chloro-7-(2,2′-dichloro-3′-(5-(((2-hydroxyethyl)amino)methyl)-6-methoxypyridin-2-yl)-[1,1′-biphenyl]-3- | |
| | yl)imidazo[1,2-a]pyridin-2-yl)methyl)amino)ethan-1-ol | |
| 384 | 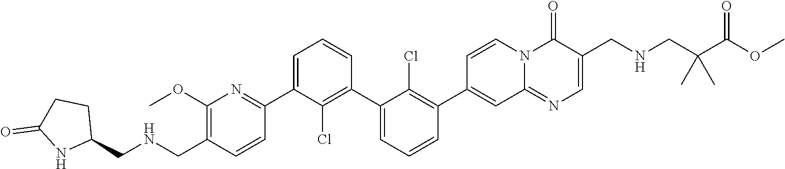 [SMILES: COC(=O)C(C)(C)CNCc1cnc2cc(-c3cccc(-c4cccc(-c5ccc(CNC[C@@H]6CCC(=O)N6)c(OC)n5)c4Cl)c3Cl)ccn2c1=O] | 0.0027 |
| | methyl (S)-3-(((8-(2,2′-dichloro-3′-(6-methoxy-5-((((5-oxopyrrolidin-2-yl)methyl)amino)methyl)pyridin-2-yl)-[1,1′-biphenyl]-3-yl)-4- | |
| | oxo-4H-pyrido[1,2-a]pyrimidin-3-yl)methyl)amino)-2,2-dimethylpropanoate | |
| 385 | 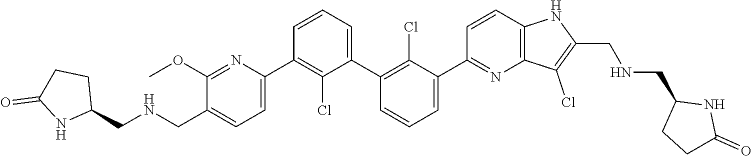 [SMILES: COc1nc(-c2cccc(-c3cccc(-c4ccc5[nH]c(CNC[C@@H]6CCC(=O)N6)c(Cl)c5n4)c3Cl)c2Cl)ccc1CNC[C@@H]1CCC(=O)N1] | 0.013 |
| | (S)-5-((((3-chloro-5-(2,2′-dichloro-3′-(6-methoxy-5-(((((S)-5-oxopyrrolidin-2-yl)methyl)amino)methyl)pyridin-2-yl)-[1,1′- | |
| | biphenyl]-3-yl)-1H-pyrrolo[3,2-b]pyridin-2-yl)methyl)amino)methyl)pyrrolidin-2-one | |
| 386 | 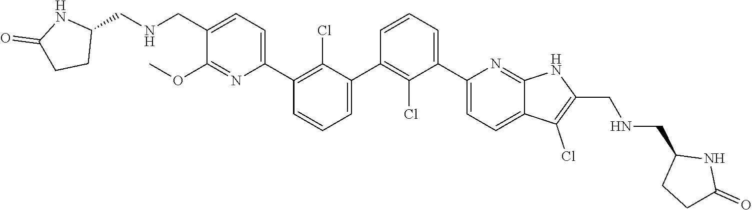 [SMILES: COc1nc(-c2cccc(-c3cccc(-c4ccc5c(Cl)c(CNC[C@@H]6CCC(=O)N6)[nH]c5n4)c3Cl)c2Cl)ccc1CNC[C@@H]1CCC(=O)N1] | 0.028 |
| | (S)-5-((((3-chloro-6-(2,2′-dichloro-3′-(6-methoxy-5-(((((S)-5-oxopyrrolidin-2-yl)methyl)amino)methyl)pyridin-2-yl)-[1,1′- | |
| | biphenyl]-3-yl)-1H-pyrrolo[2,3-b]pyridin-2-yl)methyl)amino)methyl)pyrrolidin-2-one | |
| 387 | 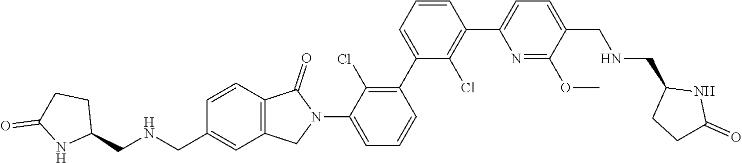 [SMILES: COc1nc(-c2cccc(-c3cccc(N4Cc5cc(CNC[C@@H]6CCC(=O)N6)ccc5C4=O)c3Cl)c2Cl)ccc1CNC[C@@H]1CCC(=O)N1] | 0.0001 |
| | 2-(2,2′-dichloro-3′-(6-methoxy-5-(((((S)-5-oxopyrrolidin-2-yl)methyl)amino)methyl)pyridin-2-yl)-[1,1′-biphenyl]-3-yl)-5- | |
| | (((((S)-5-oxopyrrolidin-2-yl)methyl)amino)methyl)isoindolin-1-one | |
| 388 | 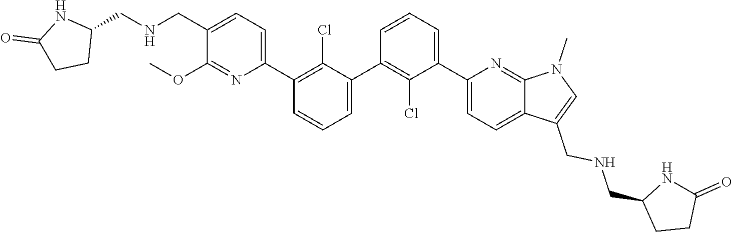 [SMILES: COc1nc(-c2cccc(-c3cccc(-c4ccc5c(CNC[C@@H]6CCC(=O)N6)cn(C)c5n4)c3Cl)c2Cl)ccc1CNC[C@@H]1CCC(=O)N1] | 0.61 |
| | (S)-5-((((6-(2,2′-dichloro-3′-(1-methyl-3-(((((S)-5-oxopyrrolidin-2-yl)methyl)amino)methyl)-1H-pyrrolo[2,3-b]pyridin-6-yl)- | |
| | [1,1′-biphenyl]-3-yl)-2-methoxypyridin-3-yl)methyl)amino)methyl)pyrrolidin-2-one | |
| 389 | 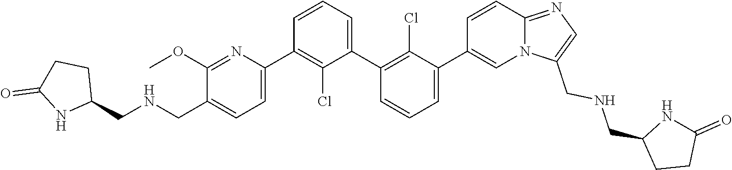 [SMILES: COc1nc(-c2cccc(-c3cccc(-c4ccc5ncc(CNC[C@@H]6CCC(=O)N6)n5c4)c3Cl)c2Cl)ccc1CNC[C@@H]1CCC(=O)N1] | 0.040 |
| | (S)-5-((((6-(2,2′-dichloro-3′-(3-(((((S)-5-oxopyrrolidin-2-yl)methyl)amino)methyl)imidazo[1,2-a]pyridin-6-yl)-[1,1′- | |
| | biphenyl]-3-yl)-2-methoxypyridin-3-yl)methyl)amino)methyl)pyrrolidin-2-one | |
| 390 | 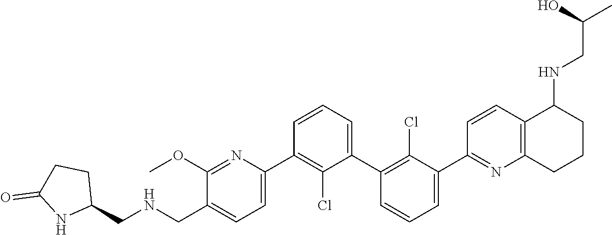 [SMILES: COc1nc(-c2cccc(-c3cccc(-c4ccc5c(n4)CCCC5NC[C@H](C)O)c3Cl)c2Cl)ccc1CNC[C@@H]1CCC(=O)N1] | 0.0065 |
| | (5S)-5-((((6-(2,2′-dichloro-3′-(5-(((S)-2-hydroxypropyl)amino)-5,6,7,8-tetrahydroquinolin-2-yl)-[1,1′- | |
| | biphenyl]-3-yl)-2-methoxypyridin-3-yl)methyl)amino)methyl)pyrrolidin-2-one | |
| | mixture of 2 diastereoisomers | |
| 391 | 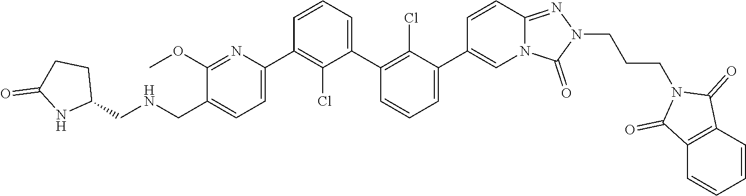 [SMILES: COc1nc(-c2cccc(-c3cccc(-c4ccc5nn(CCCN6C(=O)c7ccccc7C6=O)c(=O)n5c4)c3Cl)c2Cl)ccc1CNC[C@H]1CCC(=O)N1] | 0.065 |
| | (R)-2-(3-(6-(2,2′-dichloro-3′-(6-methoxy-5-((((5-oxopyrrolidin-2-yl)methyl)amino)methyl)pyridin-2-yl)-[1,1′-biphenyl]-3-yl)- | |
| | 3-oxo-[1,2,4]triazolo[4,3-a]pyridin-2(3H)-yl)propyl)isoindoline-1,3-dione | |
| 392 | 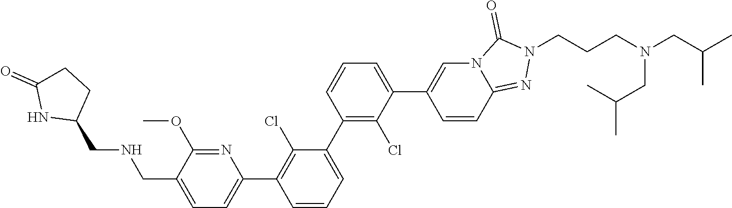 [SMILES: COc1nc(-c2cccc(-c3cccc(-c4ccc5nn(CCCN(CC(C)C)CC(C)C)c(=O)n5c4)c3Cl)c2Cl)ccc1CNC[C@@H]1CCC(=O)N1] | 0.0067 |
| | (S)-6-(2,2′-dichloro-3′-(6-methoxy-5-((((5-oxopyrrolidin-2-yl)methyl)amino)methyl)pyridin-2-yl)-[1,1′-biphenyl]-3-yl)-2-(3- | |
| | (diisobutylamino)propyl)-[1,2,4]triazolo[4,3-a]pyridin-3(2H)-one | |
| 393 | 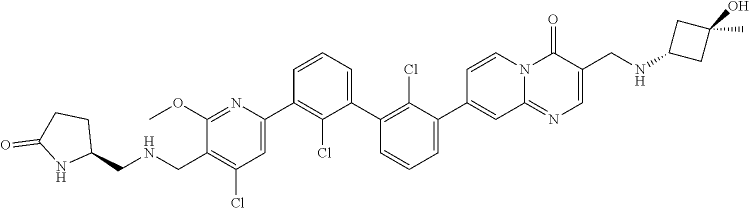 [SMILES: COc1nc(-c2cccc(-c3cccc(-c4ccn5c(=O)c(CN[C@H]6C[C@](C)(O)C6)cnc5c4)c3Cl)c2Cl)cc(Cl)c1CNC[C@@H]1CCC(=O)N1] | 0.062 |
| | 8-(2,2′-dichloro-3′-(4-chloro-6-methoxy-5-(((((S)-5-oxopyrrolidin-2-yl)methyl)amino)methyl)pyridin-2-yl)-[1,1′-biphenyl]-3- | |
| | yl)-((((1r,3r)-3-hydroxy-3-methylcyclobutyl)amino)methyl)-4H-pyrido[1,2-a]pyrimidin-4-one | |
| 394 | 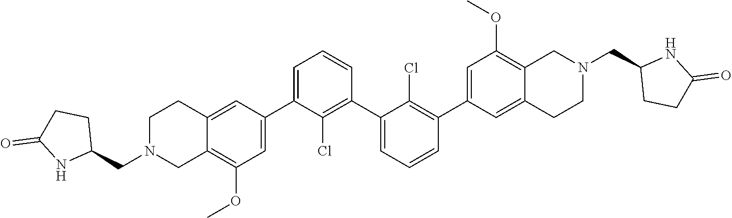 [SMILES: COc1cc(-c2cccc(-c3cccc(-c4cc5c(c(OC)c4)CN(C[C@@H]4CCC(=O)N4)CC5)c3Cl)c2Cl)cc2c1CN(C[C@@H]1CCC(=O)N1)CC2] | 0.071 |
| | (5S,5′S)-5,5′-(((2,2′-dichloro-[1,1′-biphenyl]-3,3′-diyl)bis(8-methoxy-3,4-dihydroisoquinoline-6,2(1H)- | |
| | diyl))bis(methylene))bis(pyrrolidin-2-one) | |
| 395 | 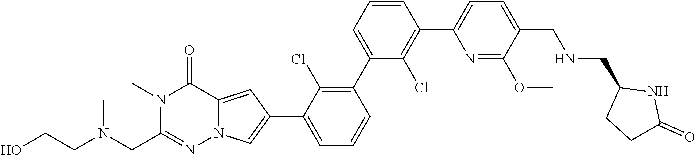 [SMILES: COc1nc(-c2cccc(-c3cccc(-c4cc5c(=O)n(C)c(CN(C)CCO)nn5c4)c3Cl)c2Cl)ccc1CNC[C@@H]1CCC(=O)N1] | 0.0067 |
| | (S)-6-(2,2′-dichloro-3′-(6-methoxy-5-((((5-oxopyrrolidin-2-yl)methyl)amino)methyl)pyridin-2-yl)-[1,1′-biphenyl]-3- | |
| | yl)-2-(((2-hydroxyethyl)(methyl)amino)methyl)-3-methylpyrrolo[2,1-f][1,2,4]triazin-4(3H)-one | |
| 396 | 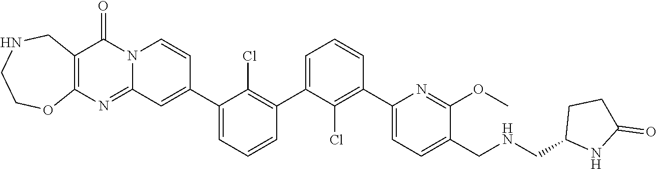 [SMILES: COc1nc(-c2cccc(-c3cccc(-c4ccn5c(=O)c6c(nc5c4)OCCNC6)c3Cl)c2Cl)ccc1CNC[C@@H]1CCC(=O)N1] | 0.015 |
| | (S)-10-(2,2′-dichloro-3′-(6-methoxy-5-((((5-oxopyrrolidin-2-yl)methyl)amino)methyl)pyridin-2-yl)-[1,1′- | |
| | biphenyl]-3-yl)-2,3,4,5-tetrahydro-6H-pyrido[1′,2′:1,2]pyrimido[5,4-f][1,4]oxazepin-6-one | |
| 397 | 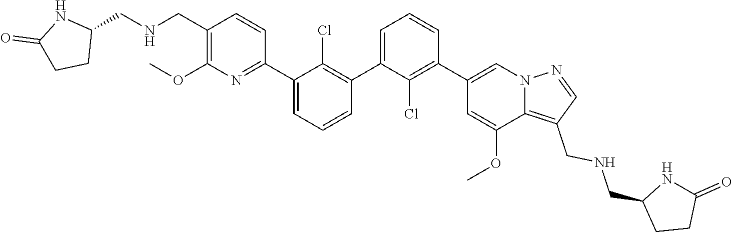 [SMILES: COc1nc(-c2cccc(-c3cccc(-c4cc(OC)c5c(CNC[C@@H]6CCC(=O)N6)cnn5c4)c3Cl)c2Cl)ccc1CNC[C@@H]1CCC(=O)N1] | 0.29 |
| | (S)-5-((((6-(2,2′-dichloro-3′-(4-methoxy-3-(((((S)-5-oxopyrrolidin-2-yl)methyl)amino)methyl)pyrazolo[1,5-a]pyridin-6-yl)- | |
| | [1,1′-biphenyl]-3-yl)-2-methoxypyridin-3-yl)methyl)amino)methyl)pyrrolidin-2-one | |
| 398 | 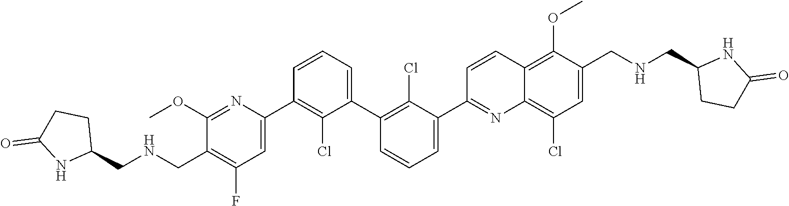 [SMILES: COc1nc(-c2cccc(-c3cccc(-c4ccc5c(OC)c(CNC[C@@H]6CCC(=O)N6)cc(Cl)c5n4)c3Cl)c2Cl)cc(F)c1CNC[C@@H]1CCC(=O)N1] | 0.028 |
| | (S)-5-((((8-chloro-2-(2,2′-dichloro-3′-(4-fluoro-6-methoxy-5-(((((S)-5-oxopyrrolidin-2-yl)methyl)amino)methyl)pyridin-2-yl)-[1,1′- | |
| | biphenyl]-3-yl)-5-methoxyquinolin-6-yl)methyl)amino)methyl)pyrrolidin-2-one | |
| 399 | 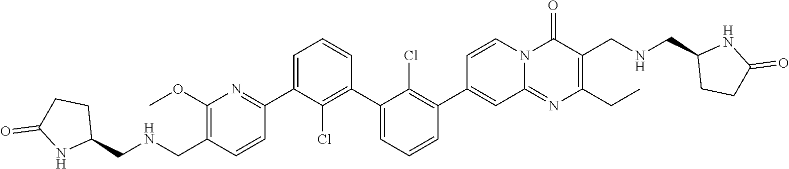 [SMILES: CCc1nc2cc(-c3cccc(-c4cccc(-c5ccc(CNC[C@@H]6CCC(=O)N6)c(OC)n5)c4Cl)c3Cl)ccn2c(=O)c1CNC[C@@H]1CCC(=O)N1] | 0.11 |
| | 8-(2,2′-dichloro-3′-(6-methoxy-5-(((((S)-5-oxopyrrolidin-2-yl)methyl)amino)methyl)pyridin-2-yl)-[1,1′-biphenyl]-3-yl)-2-ethyl-3- | |
| | (((((S)-5-oxopyrrolidin-2-yl)methyl)amino)methyl)-4H-pyrido[1,2-a]pyrimidin-4-one | |
| 400 | 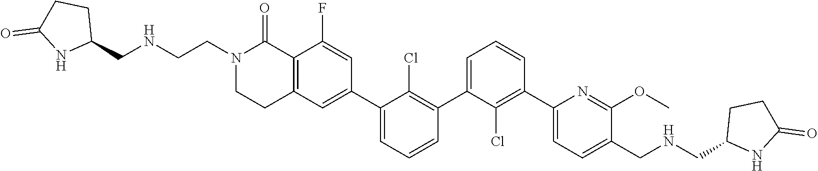 [SMILES: COc1nc(-c2cccc(-c3cccc(-c4cc(F)c5c(c4)CCN(CCNC[C@@H]4CCC(=O)N4)C5=O)c3Cl)c2Cl)ccc1CNC[C@@H]1CCC(=O)N1] | 0.55 |
| | 6-(2,2′-dichloro-3′-(6-methoxy-5-(((((S)-5-oxopyrrolidin-2-yl)methyl)amino)methyl)pyridin-2-yl)-[1,1′-biphenyl]-3-yl)-8-fluoro-2-(2- | |
| | ((((S)-5-oxopyrrolidin-2-yl)methyl)amino)ethyl)-3,4-dihydroisoquinolin-1(2H)-one | |
| 401 | 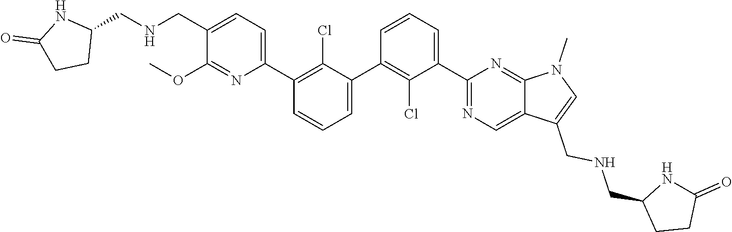 [SMILES: COc1nc(-c2cccc(-c3cccc(-c4ncc5c(CNC[C@@H]6CCC(=O)N6)cn(C)c5n4)c3Cl)c2Cl)ccc1CNC[C@@H]1CCC(=O)N1] | 0.0001 |
| | (S)-5-((((2-(2,2′-dichloro-3′-(6-methoxy-5-(((((S)-5-oxopyrrolidin-2-yl)methyl)amino)methyl)pyridin-2-yl)-[1,1′-biphenyl]-3- | |
| | yl)-7-methyl-7H-pyrrolo[2,3-d]pyrimidin-5-yl)methyl)amino)methyl)pyrrolidin-2-one | |
| 402 | 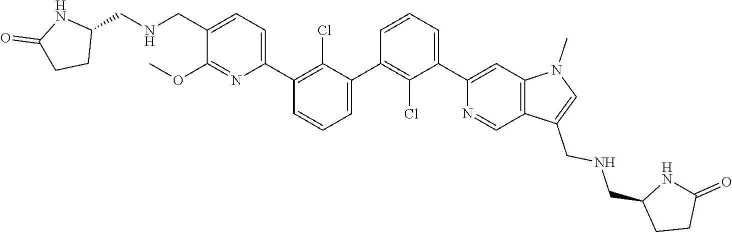 [SMILES: COc1nc(-c2cccc(-c3cccc(-c4cc5c(cn4)c(CNC[C@@H]4CCC(=O)N4)cn5C)c3Cl)c2Cl)ccc1CNC[C@@H]1CCC(=O)N1] | 0.050 |
| | (S)-5-((((6-(2,2′-dichloro-3′-(1-methyl-3-(((((S)-5-oxopyrrolidin-2-yl)methyl)amino)methyl)-1H-pyrrolo[3,2-c]pyridin-6-yl)- | |
| | [1,1′-biphenyl]-3-yl)-2-methoxypyridin-3-yl)methyl)amino)methyl)pyrrolidin-2-one | |
| 403 | 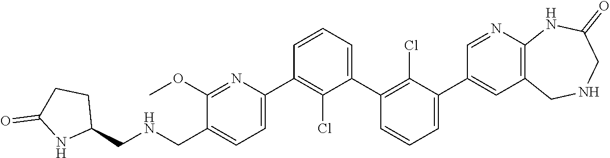 [SMILES: COc1nc(-c2cccc(-c3cccc(-c4cnc5c(c4)CNCC(=O)N5)c3Cl)c2Cl)ccc1CNC[C@@H]1CCC(=O)N1] | 0.0001 |
| | (S)-7-(2,2′-dichloro-3′-(6-methoxy-5-((((5-oxopyrrolidin-2-yl)methyl)amino)methyl)pyridin-2-yl)-[1,1′- | |
| | biphenyl]-3-yl)-1,3,4,5-tetrahydro-2H-pyrido[2,3-e][1,4]diazepin-2-one | |
| 404 | 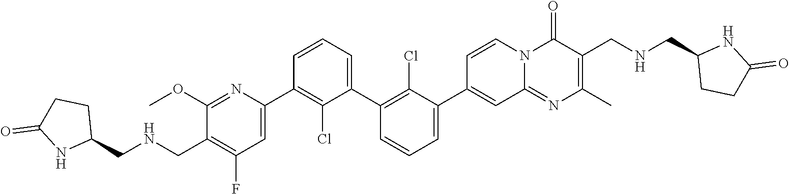 [SMILES: COc1nc(-c2cccc(-c3cccc(-c4ccn5c(=O)c(CNC[C@@H]6CCC(=O)N6)c(C)nc5c4)c3Cl)c2Cl)cc(F)c1CNC[C@@H]1CCC(=O)N1] | 0.0001 |
| | 8-(2,2′-dichloro-3′-(4-fluoro-6-methoxy-5-(((((S)-5-oxopyrrolidin-2-yl)methyl)amino)methyl)pyridin-2-yl)-[1,1′-biphenyl]-3-yl)-2- | |
| | methyl-3-(((((S)-5-oxopyrrolidin-2-yl)methyl)amino)methyl)-4H-pyrido[1,2-a]pyrimidin-4-one | |
| 405 | 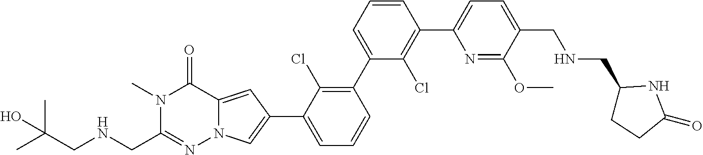 [SMILES: COc1nc(-c2cccc(-c3cccc(-c4cc5c(=O)n(C)c(CNCC(C)(C)O)nn5c4)c3Cl)c2Cl)ccc1CNC[C@@H]1CCC(=O)N1] | 0.084 |
| | (S)-6-(2,2′-dichloro-3′-(6-methoxy-5-((((5-oxopyrrolidin-2-yl)methyl)amino)methyl)pyridin-2-yl)-[1,1′-biphenyl]-3-yl)- | |
| | 2-(((2-hydroxy-2-methylpropyl)amino)methyl)-3-methylpyrrolo[2,1-f][1,2,4]triazin-4(3H)-one | |
| 406 | 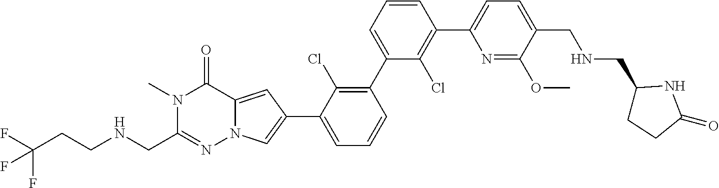 [SMILES: COc1nc(-c2cccc(-c3cccc(-c4cc5c(=O)n(C)c(CNCCC(F)(F)F)nn5c4)c3Cl)c2Cl)ccc1CNC[C@@H]1CCC(=O)N1] | 0.033 |
| | (S)-6-(2,2′-dichloro-3′-(6-methoxy-5-((((5-oxopyrrolidin-2-yl)methyl)amino)methyl)pyridin-2-yl)-[1,1′-biphenyl]-3-yl)-3- | |
| | methyl-2-(((3,3,3-trifluoropropyl)amino)methyl)pyrrolo[2,1-f][1,2,4]triazin-4(3H)-one | |
| 408 | 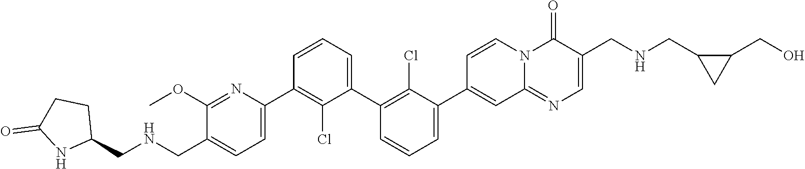 [SMILES: COc1nc(-c2cccc(-c3cccc(-c4ccn5c(=O)c(CNCC6CC6CO)cnc5c4)c3Cl)c2Cl)ccc1CNC[C@@H]1CCC(=O)N1] | 0.0011 |
| | 8-(2,2′-dichloro-3′-(6-methoxy-5-(((((S)-5-oxopyrrolidin-2-yl)methyl)amino)methyl)pyridin-2-yl)-[1,1′-biphenyl]-3-yl)-3-((((2- | |
| | (hydroxymethyl)cyclopropyl)methyl)amino)methyl)-4H-pyrido[1,2-a]pyrimidin-4-one | |
| | cis-diastereoisomer I | |
| 409 | 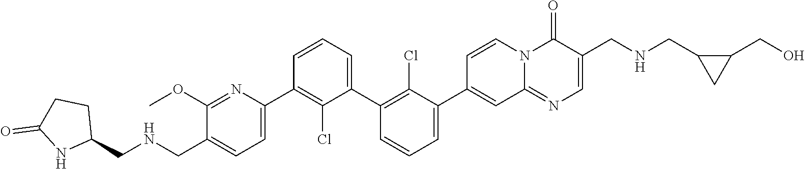 [SMILES: COc1nc(-c2cccc(-c3cccc(-c4ccn5c(=O)c(CNCC6CC6CO)cnc5c4)c3Cl)c2Cl)ccc1CNC[C@@H]1CCC(=O)N1] | 0.0002 |
| | 8-(2,2′-dichloro-3′-(6-methoxy-5-(((((S)-5-oxopyrrolidin-2-yl)methyl)amino)methyl)pyridin-2-yl)-[1,1′-biphenyl]-3-yl)-3-((((2- | |
| | (hydroxymethyl)cyclopropyl)methyl)amino)methyl)-4H-pyrido[1,2-a]pyrimidin-4-one | |
| | cis-diastereoisomer II | |
| 410 | 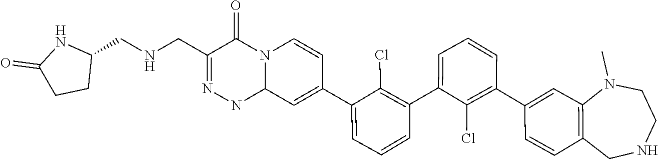 [SMILES: CN1CCNCc2ccc(-c3cccc(-c4cccc(C5=CC6NN=C(CNC[C@@H]7CCC(=O)N7)C(=O)N6C=C5)c4Cl)c3Cl)cc21] | 0.12 |
| | (S)-8-(2,2′-dichloro-3′-(1-methyl-2,3,4,5-tetrahydro-1H-benzo[e][1,4]diazepin-8-yl)-[1,1′-biphenyl]-3-yl)-3- | |
| | ((((5-oxopyrrolidin-2-yl)methyl)amino)methyl)-4H-pyrido[1,2-a]pyrimidin-4-one | |
| 411 | 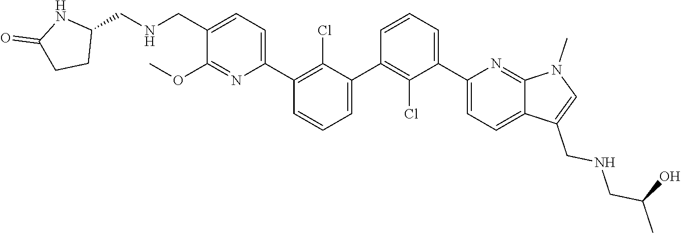 [SMILES: COc1nc(-c2cccc(-c3cccc(-c4ccc5c(CNC[C@H](C)O)cn(C)c5n4)c3Cl)c2Cl)ccc1CNC[C@@H]1CCC(=O)N1] | 0.0016 |
| | (S)-5-((((6-(2,2′-dichloro-3′-(3-((((S)-2-hydroxypropyl)amino)methyl)-1-methyl-1H-pyrrolo[2,3-b]pyridin-6-yl)- | |
| | [1,1′-biphenyl]-3-yl)-2-methoxypyridin-3-yl)methyl)amino)methyl)pyrrolidin-2-one | |
| 412 | 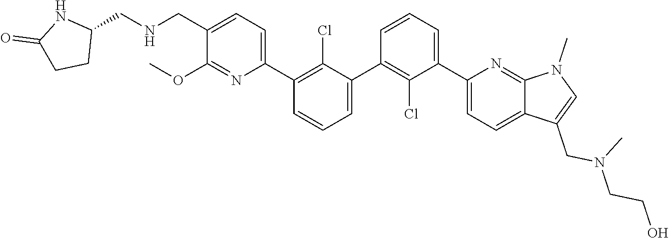 [SMILES: COc1nc(-c2cccc(-c3cccc(-c4ccc5c(CN(C)CCO)cn(C)c5n4)c3Cl)c2Cl)ccc1CNC[C@@H]1CCC(=O)N1] | 0.0052 |
| | (S)-5-((((6-(2,2′-dichloro-3′-(3-(((2-hydroxyethyl)(methyl)amino)methyl)-1-methyl-1H-pyrrolo[2,3-b]pyridin-6- | |
| | yl)-[1,1′-biphenyl]-3-yl)-2-methoxypyridin-3-yl)methyl)amino)methyl)pyrrolidin-2-one | |
| 413 | 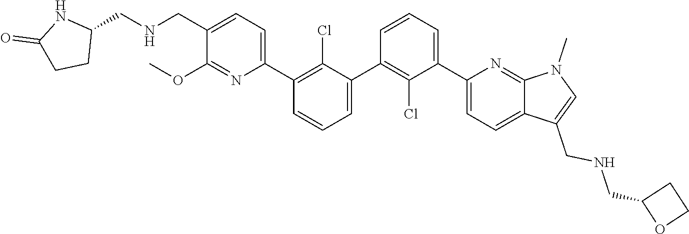 [SMILES: COc1nc(-c2cccc(-c3cccc(-c4ccc5c(CNC[C@@H]6CCO6)cn(C)c5n4)c3Cl)c2Cl)ccc1CNC[C@@H]1CCC(=O)N1] | 0.19 |
| | (S)-5-((((6-(2,2′-dichloro-3′-(1-methyl-3-(((((S)-oxetan-2-yl)methyl)amino)methyl)-1H-pyrrolo[2,3-b]pyridin-6-yl)- | |
| | [1,1′-biphenyl]-3-yl)-2-methoxypyridin-3-yl)methyl)amino)methyl)pyrrolidin-2-one | |
| 414 | 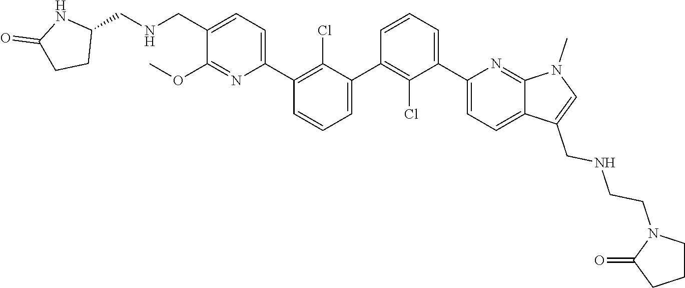 [SMILES: COc1nc(-c2cccc(-c3cccc(-c4ccc5c(CNCCN6CCCC6=O)cn(C)c5n4)c3Cl)c2Cl)ccc1CNC[C@@H]1CCC(=O)N1] | 0.17 |
| | (S)-5-((((6-(2,2′-dichloro-3′-(1-methyl-3-(((2-(2-oxopyrrolidin-1-yl)ethyl)amino)methyl)-1H-pyrrolo[2,3-b]pyridin- | |
| | 6-yl)-[1,1′-biphenyl]-3-yl)-2-methoxypyridin-3-yl)methyl)amino)methyl)pyrrolidin-2-one | |
| 415 | 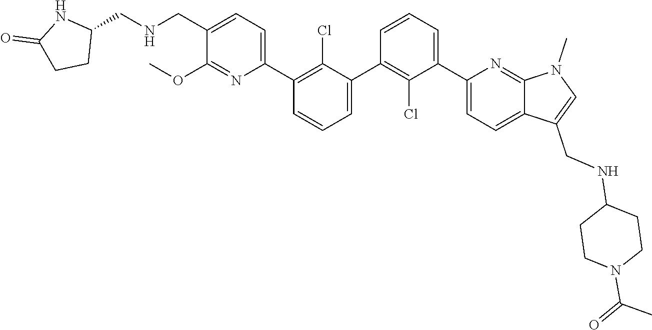 [SMILES: COc1nc(-c2cccc(-c3cccc(-c4ccc5c(CNC6CCN(C(C)=O)CC6)cn(C)c5n4)c3Cl)c2Cl)ccc1CNC[C@@H]1CCC(=O)N1] | 0.22 |
| | (S)-5-((((6-(3′-(3-(((1-acetylpiperidin-4-yl)amino)methyl)-1-methyl-1H-pyrrolo[2,3-b]pyridin-6-yl)-2,2′- | |
| | dichloro-[1,1′-biphenyl]-3-yl)-2-methoxypyridin-3-yl)methyl)amino)methyl)pyrrolidin-2-one | |
| 416 | 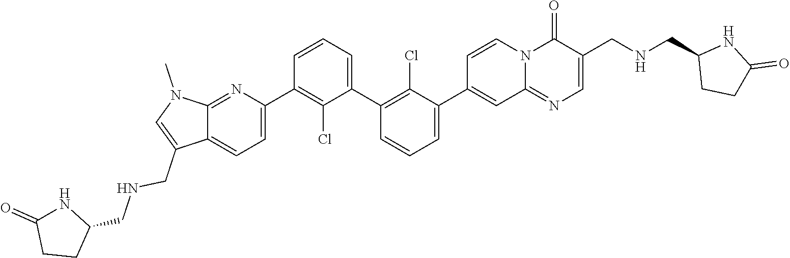 [SMILES: Cn1cc(CNC[C@@H]2CCC(=O)N2)c2ccc(-c3cccc(-c4cccc(-c5ccn6c(=O)c(CNC[C@@H]7CCC(=O)N7)cnc6c5)c4Cl)c3Cl)nc21] | 0.071 |
| | 8-(2,2′-dichloro-3′-(1-methyl-3-(((((S)-5-oxopyrrolidin-2-yl)methyl)amino)methyl)-1H-pyrrolo[2,3-b]pyridin-6-yl)-[1,1′-biphenyl]-3- | |
| | yl)-3-(((((S)-5-oxopyrrolidin-2-yl)methyl)amino)methyl)-4H-pyrido[1,2-a]pyrimidin-4-one | |
| 417 | 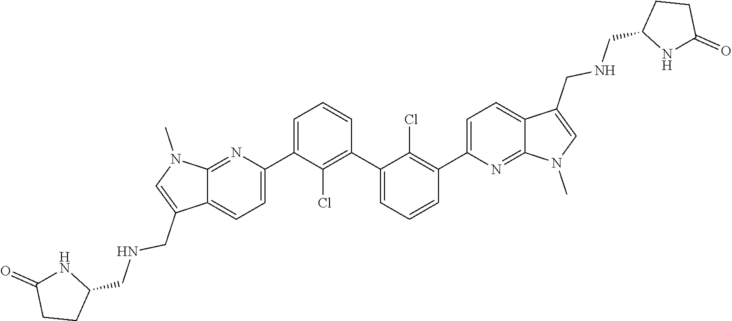 [SMILES: Cn1cc(CNC[C@@H]2CCC(=O)N2)c2ccc(-c3cccc(-c4cccc(-c5ccc6c(CNC[C@@H]7CCC(=O)N7)cn(C)c6n5)c4Cl)c3Cl)nc21] | 0.032 |
| | (5S,5′S)-5,5′-(((((2,2′-dichloro-[1,1′-biphenyl]-3,3′-diyl)bis(1-methyl-1H-pyrrolo[2,3-b]pyridine-6,3- | |
| | diyl))bis(methylene))bis(azanediyl))bis(methylene))bis(pyrrolidin-2-one) | |
| 418 | 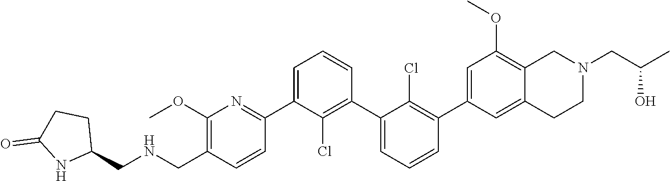 [SMILES: COc1cc(-c2cccc(-c3cccc(-c4ccc(CNC[C@@H]5CCC(=O)N5)c(OC)n4)c3Cl)c2Cl)cc2c1CN(C[C@H](C)O)CC2] | 0.023 |
| | (S)-5-((((6-(2,2′-dichloro-3′-(2-((S)-2-hydroxypropyl)-8-methoxy-1,2,3,4-tetrahydroisoquinolin-6-yl)-[1,1′- | |
| | biphenyl]-3-yl)-2-methoxypyridin-3-yl)methyl)amino)methyl)pyrrolidin-2-one | |
| 419 | 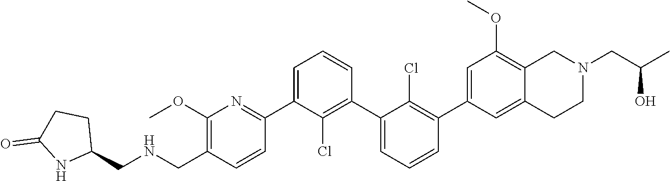 [SMILES: COc1cc(-c2cccc(-c3cccc(-c4ccc(CNC[C@@H]5CCC(=O)N5)c(OC)n4)c3Cl)c2Cl)cc2c1CN(C[C@@H](C)O)CC2] | 0.026 |
| | (S)-5-((((6-(2,2′-dichloro-3′-(2-((R)-2-hydroxypropyl)-8-methoxy-1,2,3,4-tetrahydroisoquinolin-6-yl)-[1,1′- | |
| | biphenyl]-3-yl)-2-methoxypyridin-3-yl)methyl)amino)methyl)pyrrolidin-2-one | |
| 420 | 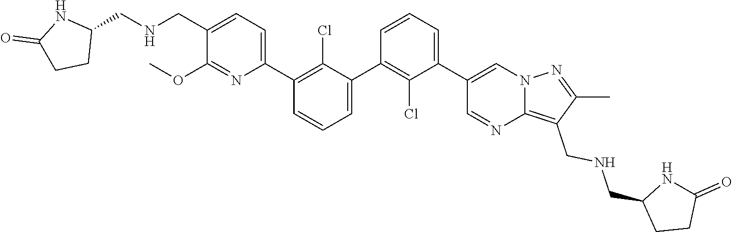 [SMILES: COc1nc(-c2cccc(-c3cccc(-c4cnc5c(CNC[C@@H]6CCC(=O)N6)c(C)nn5c4)c3Cl)c2Cl)ccc1CNC[C@@H]1CCC(=O)N1] | 0.0006 |
| | (S)-5-((((6-(2,2′-dichloro-3′-(2-methyl-3-(((((S)-5-oxopyrrolidin-2-yl)methyl)amino)methyl)pyrazolo[1,5-a]pyrimidin-6-yl)- | |
| | [1,1′-biphenyl]-3-yl)-2-methoxypyridin-3-yl)methyl)amino)methyl)pyrrolidin-2-one | |
| 421 | 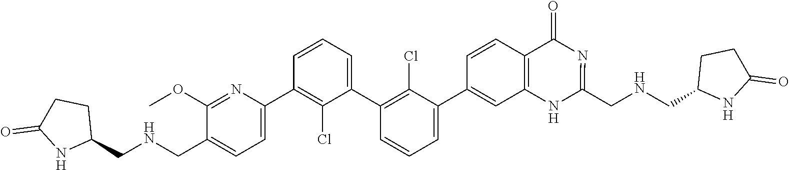 [SMILES: COc1nc(-c2cccc(-c3cccc(-c4ccc5c(=O)nc(CNC[C@@H]6CCC(=O)N6)[nH]c5c4)c3Cl)c2Cl)ccc1CNC[C@@H]1CCC(=O)N1] | 0.014 |
| | 7-(2,2′-dichloro-3′-(6-methoxy-5-(((((S)-5-oxopyrrolidin-2-yl)methyl)amino)methyl)pyridin-2-yl)-[1,1′-biphenyl]-3-yl)-2-(((((S)-5- | |
| | oxopyrrolidin-2-yl)methyl)amino)methyl)quinazolin-4(1H)-one | |
| 422 | 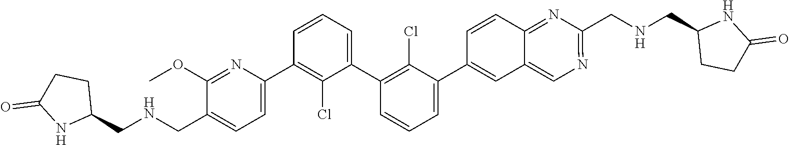 [SMILES: COc1nc(-c2cccc(-c3cccc(-c4ccc5nc(CNC[C@@H]6CCC(=O)N6)ncc5c4)c3Cl)c2Cl)ccc1CNC[C@@H]1CCC(=O)N1] | 0.0041 |
| | (S)-5-((((6-(2,2′-dichloro-3′-(2-(((((S)-5-oxopyrrolidin-2-yl)methyl)amino)methyl)quinazolin-6-yl)-[1,1′-biphenyl]-3-yl)-2- | |
| | methoxypyridin-3-yl)methyl)amino)methyl)pyrrolidin-2-one | |
| 424 | 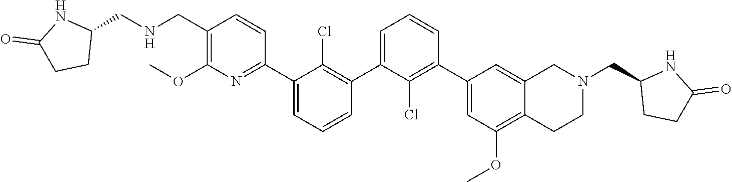 [SMILES: COc1cc(-c2cccc(-c3cccc(-c4ccc(CNC[C@@H]5CCC(=O)N5)c(OC)n4)c3Cl)c2Cl)cc2c1CCN(C[C@@H]1CCC(=O)N1)C2] | 0.027 |
| | (S)-5-((((6-(2,2′-dichloro-3′-(5-methoxy-2-(((S)-5-oxopyrrolidin-2-yl)methyl)-1,2,3,4-tetrahydroisoquinolin-7-yl)-[1,1′- | |
| | biphenyl]-3-yl)-2-methoxypyridin-3-yl)methyl)amino)methyl)pyrrolidin-2-one | |
| 425 | 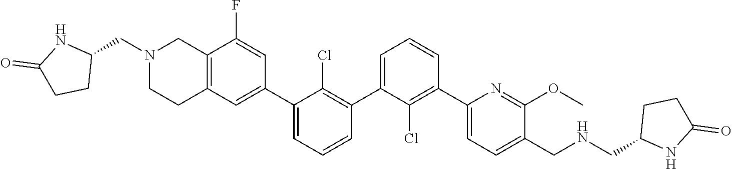 [SMILES: COc1nc(-c2cccc(-c3cccc(-c4cc(F)c5c(c4)CCN(C[C@@H]4CCC(=O)N4)C5)c3Cl)c2Cl)ccc1CNC[C@@H]1CCC(=O)N1] | 0.0083 |
| | (S)-5-((6-(2,2′-dichloro-3′-(6-methoxy-5-(((((S)-5-oxopyrrolidin-2-yl)methyl)amino)methyl)pyridin-2-yl)-[1,1′-biphenyl]- | |
| | 3-yl)-8-fluoro-3,4-dihydroisoquinolin-2(1H)-yl)methyl)pyrrolidin-2-one | |
| 426 | 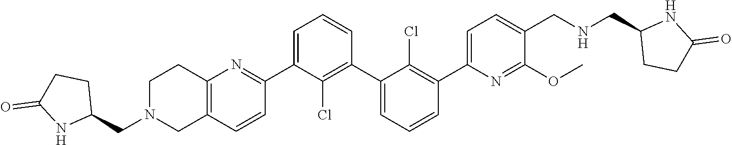 [SMILES: COc1nc(-c2cccc(-c3cccc(-c4ccc5c(n4)CCN(C[C@@H]4CCC(=O)N4)C5)c3Cl)c2Cl)ccc1CNC[C@@H]1CCC(=O)N1] | 0.0001 |
| | (S)-5-((((6-(2,2′-dichloro-3′-(6-(((S)-5-oxopyrrolidin-2-yl)methyl)-5,6,7,8-tetrahydro-1,6-naphthyridin-2-yl)-[1,1′-biphenyl]- | |
| | 3-yl)-2-methoxypyridin-3-yl)methyl)amino)methyl)pyrrolidin-2-one | |
| 427 | 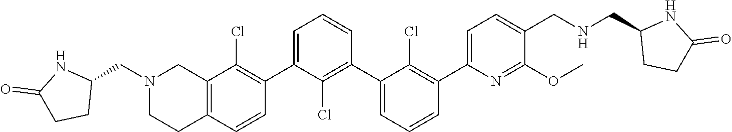 [SMILES: COc1nc(-c2cccc(-c3cccc(-c4ccc5c(c4Cl)CN(C[C@@H]4CCC(=O)N4)CC5)c3Cl)c2Cl)ccc1CNC[C@@H]1CCC(=O)N1] | 0.0052 |
| | (S)-5-((8-chloro-7-(2,2′-dichloro-3′-(6-methoxy-5-(((((S)-5-oxopyrrolidin-2-yl)methyl)amino)methyl)pyridin-2-yl)-[1,1- | |
| | biphenyl]-3-yl)-3,4-dihydroisoquinolin-2(1H)-yl)methyl)pyrrolidin-2-one | |
| 428 | 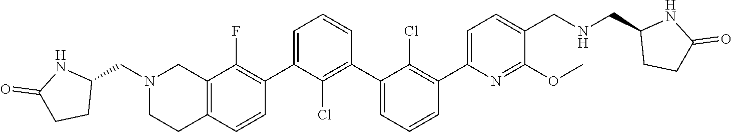 [SMILES: COc1nc(-c2cccc(-c3cccc(-c4ccc5c(c4F)CN(C[C@@H]4CCC(=O)N4)CC5)c3Cl)c2Cl)ccc1CNC[C@@H]1CCC(=O)N1] | 0.14 |
| | (S)-5-((7-(2,2′-dichloro-3′-(6-methoxy-5-(((((S)-5-oxopyrrolidin-2-yl)methyl)amino)methyl)pyridin-2-yl)-[1,1′-biphenyl]- | |
| | 3-yl)-8-fluoro-3,4-dihydroisoquinolin-2(1H)-yl)methyl)pyrrolidin-2-one | |
| 429 | 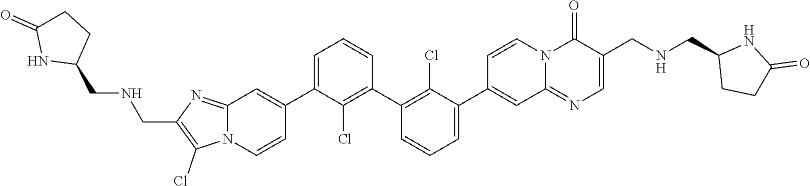 [SMILES: O=C1CC[C@@H](CNCc2nc3cc(-c4cccc(-c5cccc(-c6ccn7c(=O)c(CNC[C@@H]8CCC(=O)N8)cnc7c6)c5Cl)c4Cl)ccn3c2Cl)N1] | 0.24 |
| | 8-(2,2′-dichloro-3′-(3-chloro-2-(((((S)-5-oxopyrrolidin-2-yl)methyl)amino)methyl)imidazo[1,2-a]pyridin-7-yl)-[1,1′-biphenyl]-3-yl)-3- | |
| | (((((S)-5-oxopyrrolidin-2-yl)methyl)amino)methyl)-4H-pyrido[1,2-a]pyrimidin-4-one | |
| 430 | 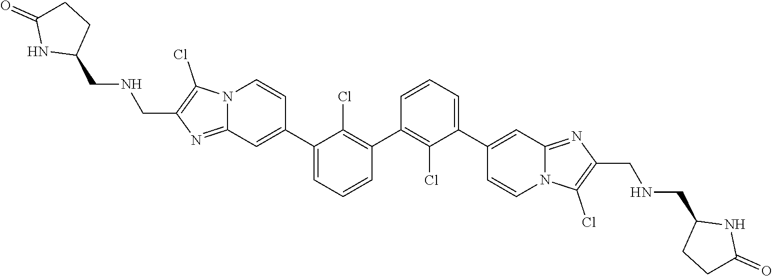 [SMILES: O=C1CC[C@@H](CNCc2nc3cc(-c4cccc(-c5cccc(-c6ccn7c(Cl)c(CNC[C@@H]8CCC(=O)N8)nc7c6)c5Cl)c4Cl)ccn3c2Cl)N1] | 0.17 |
| | (5S,5′S)-5,5′-(((((2,2′-dichloro-[1,1′-biphenyl]-3,3′-diyl)bis(3-chloroimidazo[1,2-a]pyridine-7,2- | |
| | diyl))bis(methylene))bis(azanediyl))bis(methylene))bis(pyrrolidin-2-one) | |
| 431 | 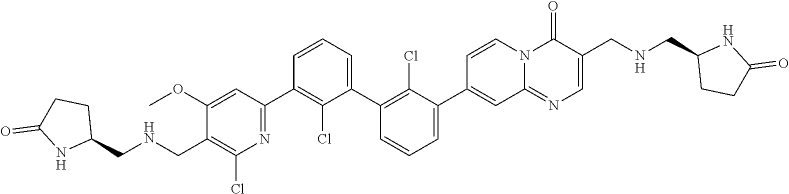 [SMILES: COc1cc(-c2cccc(-c3cccc(-c4ccn5c(=O)c(CNC[C@@H]6CCC(=O)N6)cnc5c4)c3Cl)c2Cl)nc(Cl)c1CNC[C@@H]1CCC(=O)N1] | 0.18 |
| | 8-(2,2′-dichloro-3′-(6-chloro-4-methoxy-5-(((((S)-5-oxopyrrolidin-2-yl)methyl)amino)methyl)pyridin-2-yl)-[1,1′-biphenyl]-3-yl)-3- | |
| | (((((S)-5-oxopyrrolidin-2-yl)methyl)amino)methyl)-4H-pyrido[1,2-a]pyrimidin-4-one | |
| 432 | 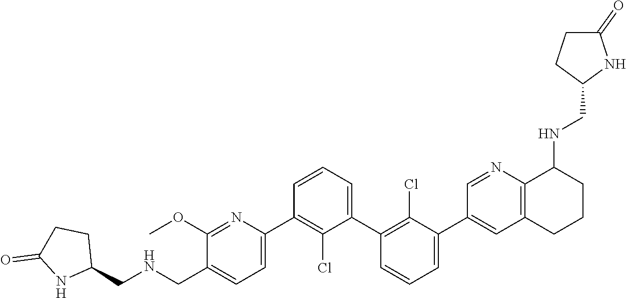 [SMILES: COc1nc(-c2cccc(-c3cccc(-c4cnc5c(c4)CCCC5NC[C@@H]4CCC(=O)N4)c3Cl)c2Cl)ccc1CNC[C@@H]1CCC(=O)N1] | 0.27 |
| | (5S)-5-(((3-(2,2′-dichloro-3′-(6-methoxy-5-(((((S)-5-oxopyrrolidin-2-yl)methyl)amino)methyl)pyridin-2-yl)- | |
| | [1,1′-biphenyl]-3-yl)-5,6,7,8-tetrahydroquinolin-8-yl)amino)methyl)pyrrolidin-2-one | |
| 433 | 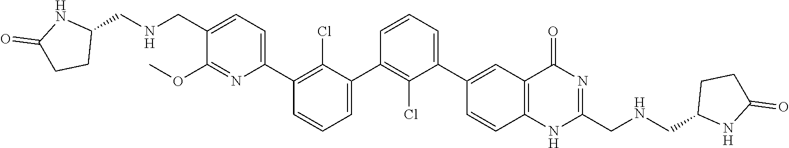 [SMILES: COc1nc(-c2cccc(-c3cccc(-c4ccc5[nH]c(CNC[C@@H]6CCC(=O)N6)nc(=O)c5c4)c3Cl)c2Cl)ccc1CNC[C@@H]1CCC(=O)N1] | 0.071 |
| | 6-(2,2′-dichloro-3′-(6-methoxy-5-(((((S)-5-oxopyrrolidin-2-yl)methyl)amino)methyl)pyridin-2-yl)-[1,1′-biphenyl]-3-yl)-2-(((((S)-5- | |
| | oxopyrrolidin-2-yl)methyl)amino)methyl)quinazolin-4(1H)-one | |
| 434 | 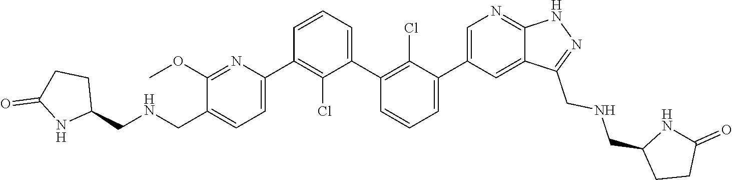 [SMILES: COc1nc(-c2cccc(-c3cccc(-c4cnc5[nH]nc(CNC[C@@H]6CCC(=O)N6)c5c4)c3Cl)c2Cl)ccc1CNC[C@@H]1CCC(=O)N1] | 0.032 |
| | (S)-5-((((6-(2,2′-dichloro-3′-(3-(((((S)-5-oxopyrrolidin-2-yl)methyl)amino)methyl)-1H-pyrazolo[3,4-b]pyridin-5-yl)-[1,1′- | |
| | biphenyl]-3-yl)-2-methoxypyridin-3-yl)methyl)amino)methyl)pyrrolidin-2-one | |
| 435 | 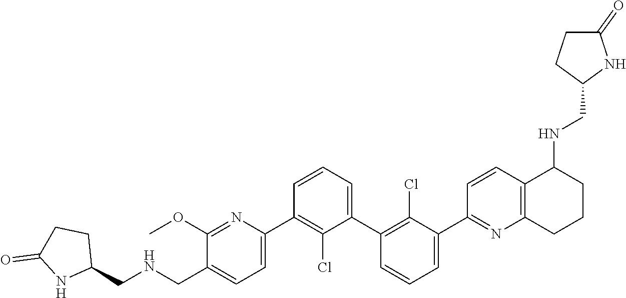 [SMILES: COc1nc(-c2cccc(-c3cccc(-c4ccc5c(n4)CCCC5NC[C@@H]4CCC(=O)N4)c3Cl)c2Cl)ccc1CNC[C@@H]1CCC(=O)N1] | 0.023 |
| | (5S)-5-((((6-(2,2′-dichloro-3′-(5-((((S)-5-oxopyrrolidin-2-yl)methyl)amino)-5,6,7,8-tetrahydroquinolin-2- | |
| | yl)-[1,1′-biphenyl]-3-yl)-2-methoxypyridin-3-yl)methyl)amino)methyl)pyrrolidin-2-one | |
| 436 | 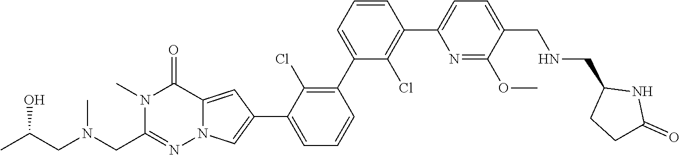 [SMILES: COc1nc(-c2cccc(-c3cccc(-c4cc5c(=O)n(C)c(CN(C)C[C@H](C)O)nn5c4)c3Cl)c2Cl)ccc1CNC[C@@H]1CCC(=O)N1] | 0.026 |
| | 6-(2,2′-dichloro-3′-(6-methoxy-5-(((((S)-5-oxopyrrolidin-2-yl)methyl)amino)methyl)pyridin-2-yl)-[1,1′-biphenyl]-3- | |
| | yl)-2-((((S)-2-hydroxypropyl)(methyl)amino)methyl)-3-methylpyrrolo[2,1-f][1,2,4]triazin-4(3H)-one | |
| 437 | 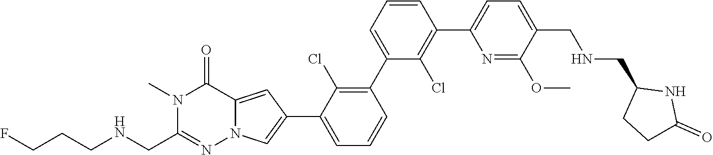 [SMILES: COc1nc(-c2cccc(-c3cccc(-c4cc5c(=O)n(C)c(CNCCCF)nn5c4)c3Cl)c2Cl)ccc1CNC[C@@H]1CCC(=O)N1] | 0.052 |
| | (S)-6-(2,2′-dichloro-3′-(6-methoxy-5-((((5-oxopyrrolidin-2-yl)methyl)amino)methyl)pyridin-2-yl)-[1,1′-biphenyl]-3-yl)-2- | |
| | (((3-fluoropropyl)amino)methyl)-3-methylpyrrolo[2,1-f][1,2,4]triazin-4(3H)-one | |
| 438 | 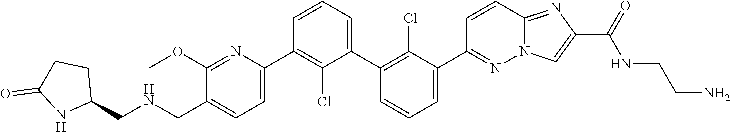 [SMILES: COc1nc(-c2cccc(-c3cccc(-c4ccc5nc(C(=O)NCCN)cn5n4)c3Cl)c2Cl)ccc1CNC[C@@H]1CCC(=O)N1] | 0.014 |
| | (S)-N-(2-aminoethyl)-6-(2,2′-dichloro-3′-(6-methoxy-5-((((5-oxopyrrolidin-2-yl)methyl)amino)methyl)pyridin-2-yl)-[1,1′- | |
| | biphenyl]-3-yl)imidazo[1,2-b]pyridazine-2-carboxamide | |
| 439 | 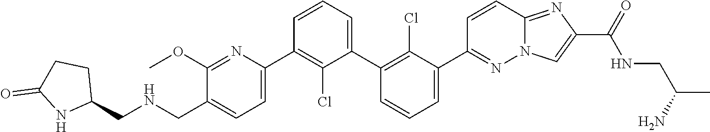 [SMILES: COc1nc(-c2cccc(-c3cccc(-c4ccc5nc(C(=O)NC[C@H](C)N)cn5n4)c3Cl)c2Cl)ccc1CNC[C@@H]1CCC(=O)N1] | 0.020 |
| | N-((S)-2-aminopropyl)-6-(2,2′-dichloro-3′-(6-methoxy-5-(((((S)-5-oxopyrrolidin-2-yl)methyl)amino)methyl)pyridin-2- | |
| | yl)-[1,1′-biphenyl]-3-yl)imidazo[1,2-b]pyridazine-2-carboxamide | |
| 440 | 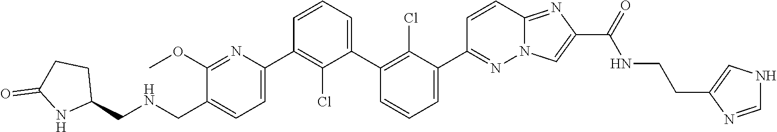 [SMILES: COc1nc(-c2cccc(-c3cccc(-c4ccc5nc(C(=O)NCCc6c[nH]cn6)cn5n4)c3Cl)c2Cl)ccc1CNC[C@@H]1CCC(=O)N1] | 0.027 |
| | (S)-N-(2-(1H-imidazol-4-yl)ethyl)-6-(2,2′-dichloro-3′-(6-methoxy-5-((((5-oxopyrrolidin-2-yl)methyl)amino)methyl)pyridin-2-yl)- | |
| | [1,1′-biphenyl]-3-yl)imidazo[1,2-b]pyridazine-2-carboxamid | |
| 441 | 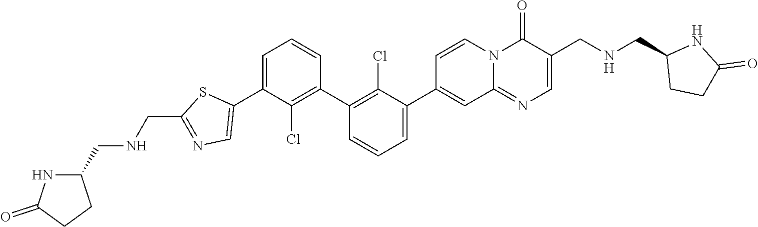 [SMILES: O=C1CC[C@@H](CNCc2ncc(-c3cccc(-c4cccc(-c5ccn6c(=O)c(CNC[C@@H]7CCC(=O)N7)cnc6c5)c4Cl)c3Cl)s2)N1] | 0.79 |
| | 8-(2,2′-dichloro-3′-(2-(((((S)-5-oxopyrrolidin-2-yl)methyl)amino)methyl)thiazol-5-yl)-[1,1′-biphenyl]-3-yl)-3-(((((S)-5- | |
| | oxopyrrolidin-2-yl)methyl)amino)methyl)-4H-pyrido[1,2-a]pyrimidin-4-one | |
| 442 | 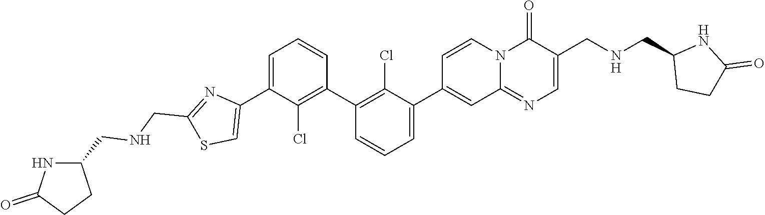 [SMILES: O=C1CC[C@@H](CNCc2nc(-c3cccc(-c4cccc(-c5ccn6c(=O)c(CNC[C@@H]7CCC(=O)N7)cnc6c5)c4Cl)c3Cl)cs2)N1] | 2.3 |
| | 8-(2,2′-dichloro-3′-(2-(((((S)-5-oxopyrrolidin-2-yl)methyl)amino)methyl)thiazol-4-yl)-[1,1′-biphenyl]-3-yl)-3-(((((S)-5- | |
| | oxopyrrolidin-2-yl)methyl)amino)methyl)-4H-pyrido[1,2-a]pyrimidin-4-one | |
| 443 | 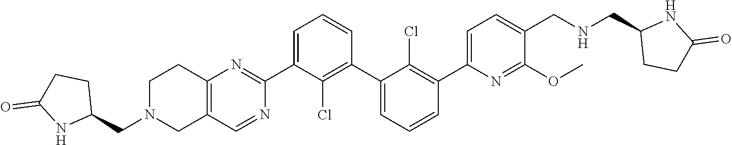 [SMILES: COc1nc(-c2cccc(-c3cccc(-c4ncc5c(n4)CCN(C[C@@H]4CCC(=O)N4)C5)c3Cl)c2Cl)ccc1CNC[C@@H]1CCC(=O)N1] | 0.0001 |
| | (S)-5-((((6-(2,2′-dichloro-3′-(6-(((S)-5-oxopyrrolidin-2-yl)methyl)-5,6,7,8-tetrahydropyrido[4,3-d]pyrimidin-2-yl)-[1,1′- | |
| | biphenyl]-3-yl)-2-methoxypyridin-3-yl)methyl)amino)methyl)pyrrolidin-2-one | |
| 444 | 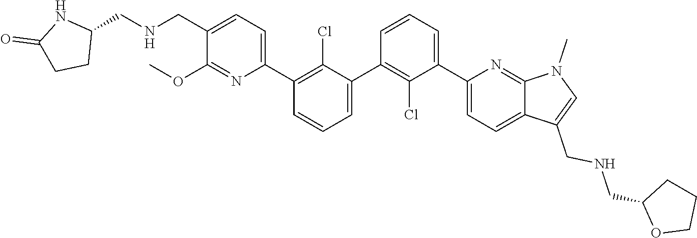 [SMILES: COc1nc(-c2cccc(-c3cccc(-c4ccc5c(CNC[C@@H]6CCCO6)cn(C)c5n4)c3Cl)c2Cl)ccc1CNC[C@@H]1CCC(=O)N1] | 0.92 |
| | (S)-5-((((6-(2,2′-dichloro-3′-(1-methyl-3-(((((S)-tetrahydrofuran-2-yl)methyl)amino)methyl)-1H-pyrrolo[2,3-b]pyridin- | |
| | 6-yl)-[1,1′-biphenyl]-3-yl)-2-methoxypyridin-3-yl)methyl)amino)methyl)pyrrolidin-2-one | |
| 445 | 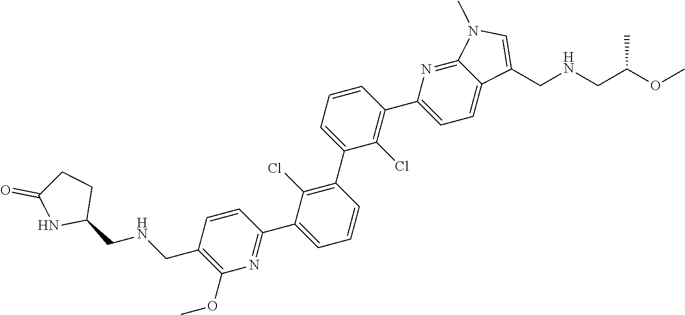 [SMILES: COc1nc(-c2cccc(-c3cccc(-c4ccc5c(CNC[C@H](C)OC)cn(C)c5n4)c3Cl)c2Cl)ccc1CNC[C@@H]1CCC(=O)N1] | 0.46 |
| | (S)-5-((((6-(2,2′-dichloro-3′-(3-((((S)-2-methoxypropyl)amino)methyl)-1-methyl-1H-pyrrolo[2,3-b]pyridin-6-yl)-[1,1′- | |
| | biphenyl]-3-yl)-2-methoxypyridin-3-yl)methyl)amino)methyl)pyrrolidin-2-one | |
| 446 | 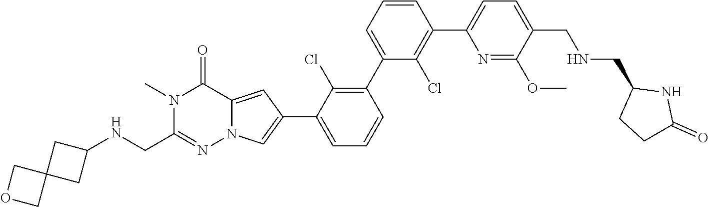 [SMILES: COc1nc(-c2cccc(-c3cccc(-c4cc5c(=O)n(C)c(CNC6CC7(COC7)C6)nn5c4)c3Cl)c2Cl)ccc1CNC[C@@H]1CCC(=O)N1] | 0.16 |
| | (S)-2-(((2-oxaspiro[3.3]heptan-6-yl)amino)methyl)-6-(2,2′-dichloro-3′-(6-methoxy-5-((((5-oxopyrrolidin-2- | |
| | yl)methyl)amino)methyl)pyridin-2-yl)-[1,1′-biphenyl]-3-yl)-3-methylpyrrolo[2,1-f][1,2,4]triazin-4(3H)-one | |
| 447 | 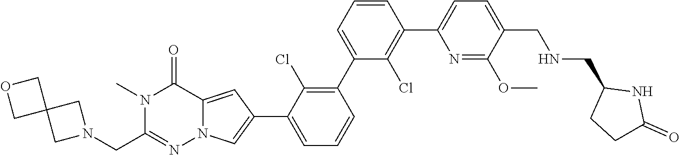 [SMILES: COc1nc(-c2cccc(-c3cccc(-c4cc5c(=O)n(C)c(CN6CC7(COC7)C6)nn5c4)c3Cl)c2Cl)ccc1CNC[C@@H]1CCC(=O)N1] | 0.0050 |
| | (S)-2-((2-oxa-6-azaspiro[3.3]heptan-6-yl)methyl)-6-(2,2′-dichloro-3′-(6-methoxy-5((((5-oxopyrrolidin-2- | |
| | yl)methyl)amino)methyl)pyridin-2-yl)-[1,1′-biphenyl]-3-yl)-3-methylpyrrolo[2,1-f][1,2,4]triazin-4(3H)-one | |
| 448 | 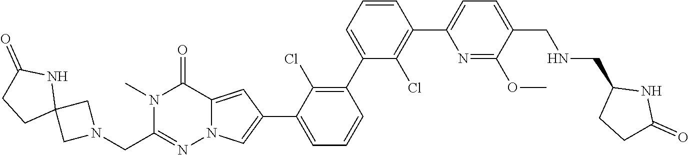 [SMILES: COc1nc(-c2cccc(-c3cccc(-c4cc5c(=O)n(C)c(CN6CC7(CCC(=O)N7)C6)nn5c4)c3Cl)c2Cl)ccc1CNC[C@@H]1CCC(=O)N1] | 0.0068 |
| | (S)-6-(2,2′-dichloro-3′-(6-methoxy-5-((((5-oxopyrrolidin-2-yl)methyl)amino)methyl)pyridin-2-yl)-[1,1′-biphenyl]-3- | |
| | yl)-3-methyl-2-((6-oxo-2,5-diazaspiro[3.4]octan-2-yl)methyl)pyrrolo[2,1-f][1,2,4]triazin-4(3H)-one | |
| 449 | 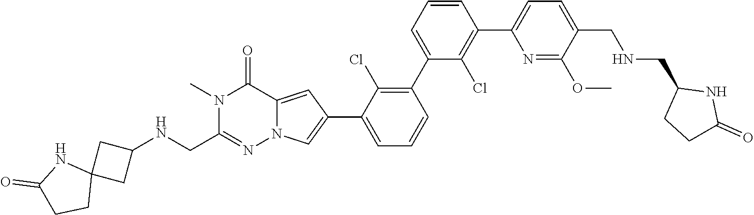 [SMILES: COc1nc(-c2cccc(-c3cccc(-c4cc5c(=O)n(C)c(CNC6CC7(CCC(=O)N7)C6)nn5c4)c3Cl)c2Cl)ccc1CNC[C@@H]1CCC(=O)N1] | 0.45 |
| | (S)-6-(2,2′-dichloro-3′-(6-methoxy-5-((((5-oxopyrrolidin-2-yl)methyl)amino)methyl)pyridin-2-yl)-[1,1′-biphenyl]-3-yl)-3-methyl- | |
| | 2-(((6-oxo-5-azaspiro[3.4]octan-2-yl)amino)methyl)pyrrolo[2,1-f][1,2,4]triazin-4(3H)-one | |
| 450 | 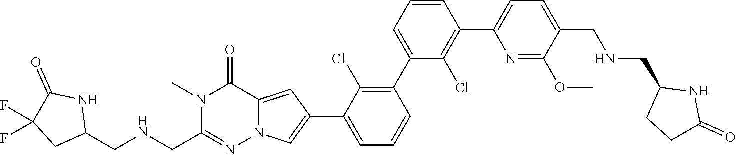 [SMILES: COc1nc(-c2cccc(-c3cccc(-c4cc5c(=O)n(C)c(CNCC6CC(F)(F)C(=O)N6)nn5c4)c3Cl)c2Cl)ccc1CNC[C@@H]1CCC(=O)N1] | 0.012 |
| | 6-(2,2′-dichloro-3′-(6-methoxy-5-(((((S)-5-oxopyrrolidin-2-yl)methyl)amino)methyl)pyridin-2-yl)-[1,1′-biphenyl]-3-yl)-2- | |
| | ((((4,4-difluoro-5-oxopyrrolidin-2-yl)methyl)amino)methyl)-3-methylpyrrolo[2,1-f][1,2,4]triazin-4(3H)-one | |
| 451 | 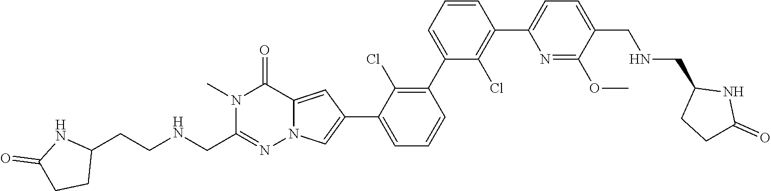 [SMILES: COc1nc(-c2cccc(-c3cccc(-c4cc5c(=O)n(C)c(CNCCC6CCC(=O)N6)nn5c4)c3Cl)c2Cl)ccc1CNC[C@@H]1CCC(=O)N1] | 0.20 |
| | 6-(2,2′-dichloro-3′-(6-methoxy-5-(((((S)-5-oxopyrrolidin-2-yl)methyl)amino)methyl)pyridin-2-yl)-[1,1′-biphenyl]-3-yl)-3-methyl-2- | |
| | (((2-(5-oxopyrrolidin-2-yl)ethyl)amino)methyl)pyrrolo[2,1-f][1,2,4]triazin-4(3H)-one | |
| 452 | 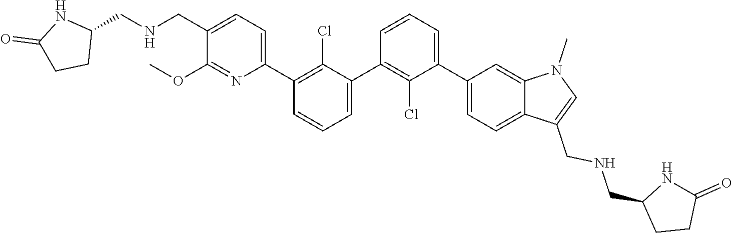 [SMILES: COc1nc(-c2cccc(-c3cccc(-c4ccc5c(CNC[C@@H]6CCC(=O)N6)cn(C)c5c4)c3Cl)c2Cl)ccc1CNC[C@@H]1CCC(=O)N1] | 0.068 |
| | (S)-5-((((6-(2,2′-dichloro-3′-(1-methyl-3-(((((S)-5-oxopyrrolidin-2-yl)methyl)amino)methyl)-1H-indol-6-yl)-[1,1′-biphenyl]- | |
| | 3-yl)-2-methoxypyridin-3-yl)methyl)amino)methyl)pyrrolidin-2-one | |
| 453 | 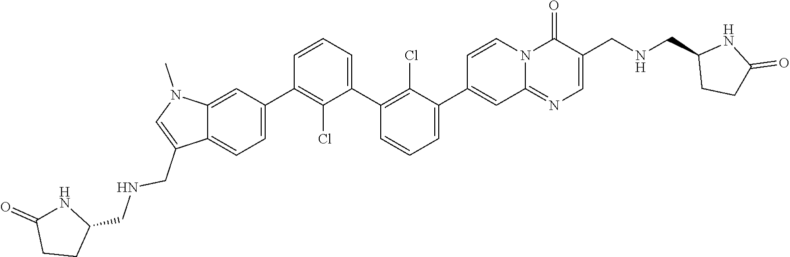 [SMILES: Cn1cc(CNC[C@@H]2CCC(=O)N2)c2ccc(-c3cccc(-c4cccc(-c5ccn6c(=O)c(CNC[C@@H]7CCC(=O)N7)cnc6c5)c4Cl)c3Cl)cc21] | 1.2 |
| | 8-(2,2′-dichloro-3′-(1-methyl-3-(((((S)-5-oxopyrrolidin-2-yl)methyl)amino)methyl)-1H-indol-6-yl)-[1,1′-biphenyl]-3-yl)-3-(((((S)-5- | |
| | oxopyrrolidin-2-yl)methyl)amino)methyl)-4H-pyrido[1,2-a]pyrimidin-4-one | |
| 454 | 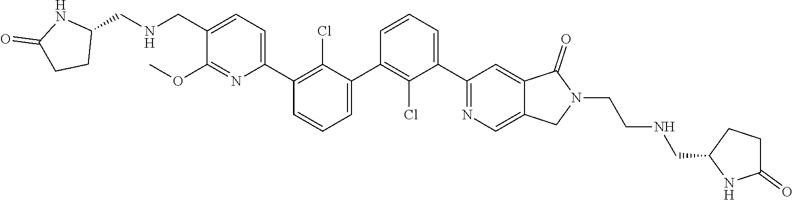 [SMILES: COc1nc(-c2cccc(-c3cccc(-c4cc5c(cn4)CN(CCNC[C@@H]4CCC(=O)N4)C5=O)c3Cl)c2Cl)ccc1CNC[C@@H]1CCC(=O)N1] | 0.0001 |
| | 6-(2,2′-dichloro-3′-(6-methoxy-5-(((((S)-5-oxopyrrolidin-2-yl)methyl)amino)methyl)pyridin-2-yl)-[1,1′-biphenyl]-3-yl)-2-(2-((((S)-5- | |
| | oxopyrrolidin-2-yl)methyl)amino)ethyl)-2,3-dihydro-1H-pyrrolo[3,4-c]pyridin-1-one | |
| 455 | 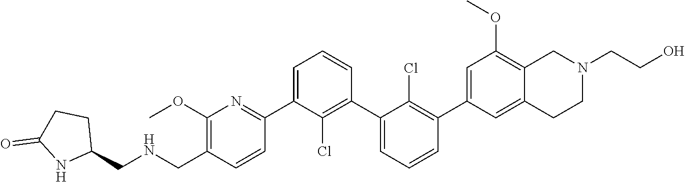 [SMILES: COc1cc(-c2cccc(-c3cccc(-c4ccc(CNC[C@@H]5CCC(=O)N5)c(OC)n4)c3Cl)c2Cl)cc2c1CN(CCO)CC2] | 0.012 |
| | (S)-5-((((6-(2,2′-dichloro-3′-(2-(2-hydroxyethyl)-8-methoxy-1,2,3,4-tetrahydroisoquinolin-6-yl)-[1,1′-biphenyl]-3-yl)- | |
| | 2-methoxypyridin-3-yl)methyl)amino)methyl)pyrrolidin-2-one | |
| 456 | 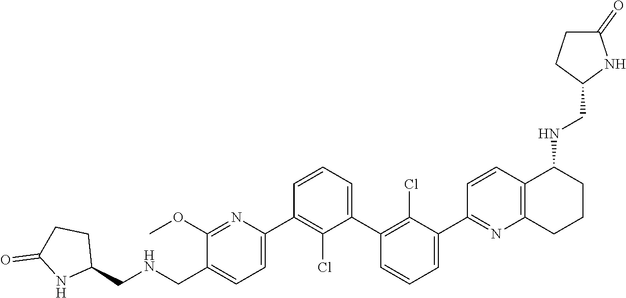 [SMILES: COc1nc(-c2cccc(-c3cccc(-c4ccc5c(n4)CCC[C@H]5NC[C@@H]4CCC(=O)N4)c3Cl)c2Cl)ccc1CNC[C@@H]1CCC(=O)N1] | 0.0002 |
| | (S)-5-((((6-(2,2′-dichloro-3′-((R)-5-((((S)-5-oxopyrrolidin-2-yl)methyl)amino)-5,6,7,8-tetrahydroquinolin-2- | |
| | yl)-[1,1′-biphenyl]-3-yl)-2-methoxypyridin-3-yl)methyl)amino)methyl)pyrrolidin-2-one | |
| 457 | 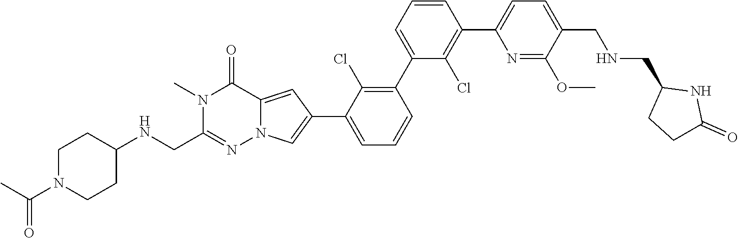 [SMILES: COc1nc(-c2cccc(-c3cccc(-c4cc5c(=O)n(C)c(CNC6CCN(C(C)=O)CC6)nn5c4)c3Cl)c2Cl)ccc1CNC[C@@H]1CCC(=O)N1] | 0.19 |
| | (S)-2-(((1-acetylpiperidin-4-yl)amino)methyl)-6-(2,2′-dichloro-3′-(6-methoxy-5-((((5-oxopyrrolidin-2- | |
| | yl)methyl)amino)methyl)pyridin-2-yl)-[1,1′-biphenyl]-3-yl)-3-methylpyrrolo[2,1-f][1,2,4]triazin-4(3H)-one | |
| 458 | 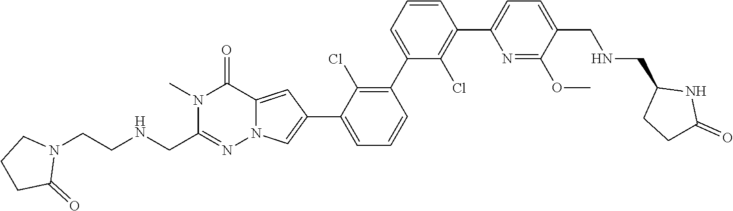 [SMILES: COc1nc(-c2cccc(-c3cccc(-c4cc5c(=O)n(C)c(CNCCN6CCCC6=O)nn5c4)c3Cl)c2Cl)ccc1CNC[C@@H]1CCC(=O)N1] | 0.24 |
| | (S)-6-(2,2′-dichloro-3′-(6-methoxy-5-((((5-oxopyrrolidin-2-yl)methyl)amino)methyl)pyridin-2-yl)-[1,1′-biphenyl]-3-yl)-3- | |
| | methyl-2-(((2-(2-oxopyrrolidin-1-yl)ethyl)amino)methyl)pyrrolo[2,1-f][1,2,4]triazin-4(3H)-one | |
| 459 | 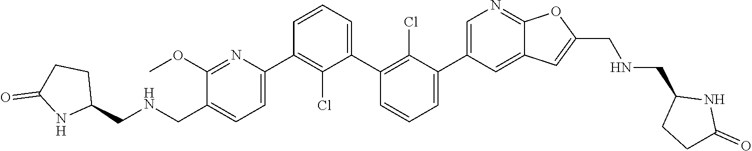 [SMILES: COc1nc(-c2cccc(-c3cccc(-c4cnc5oc(CNC[C@@H]6CCC(=O)N6)cc5c4)c3Cl)c2Cl)ccc1CNC[C@@H]1CCC(=O)N1] | 0.20 |
| | (S)-5-((((6-(2,2′-dichloro-3′-(2-(((((S)-5-oxopyrrolidin-2-yl)methyl)amino)methyl)furo[2,3-b]pyridin-5-yl)-[1,1′-biphenyl]-3-yl)- | |
| | 2-methoxypyridin-3-yl)methyl)amino)methyl)pyrrolidin-2-one | |
| 460 | 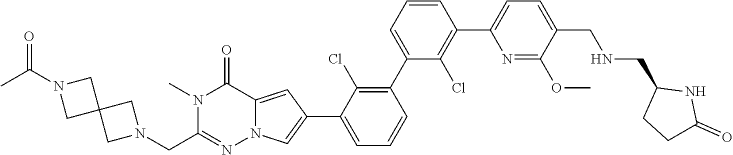 [SMILES: COc1nc(-c2cccc(-c3cccc(-c4cc5c(=O)n(C)c(CN6CC7(C6)CN(C(C)=O)C7)nn5c4)c3Cl)c2Cl)ccc1CNC[C@@H]1CCC(=O)N1] | 0.0099 |
| | (S)-2-((6-acetyl-2,6-diazaspiro[3.3]heptan-2-yl)methyl)-6-(2,2′-dichloro-3′-(6-methoxy-5-((((5-oxopyrrolidin-2- | |
| | yl)methyl)amino)methyl)pyridin-2-yl)-[1,1′-biphenyl]-3-yl)-3-methylpyrrolo[2,1-f][1,2,4]triazin-4(3H)-one | |
| 461 | 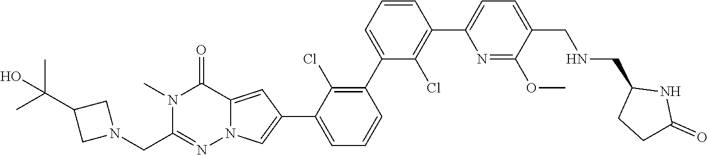 [SMILES: COc1nc(-c2cccc(-c3cccc(-c4cc5c(=O)n(C)c(CN6CC(C(C)(C)O)C6)nn5c4)c3Cl)c2Cl)ccc1CNC[C@@H]1CCC(=O)N1] | 0.013 |
| | (S)-6-(2,2′-dichloro-3′-(6-methoxy-5-((((5-oxopyrrolidin-2-yl)methyl)amino)methyl)pyridin-2-yl)-[1,1′-biphenyl]-3-yl)-2- | |
| | ((3-(2-hydroxypropan-2-yl)azetidin-1-yl)methyl)-3-methylpyrrolo[2,1-f][1,2,4]triazin-4(3H)-one | |
| 462 | 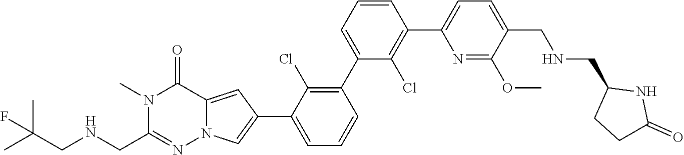 [SMILES: COc1nc(-c2cccc(-c3cccc(-c4cc5c(=O)n(C)c(CNCC(C)(C)F)nn5c4)c3Cl)c2Cl)ccc1CNC[C@@H]1CCC(=O)N1] | 0.058 |
| | (S)-6-(2,2′-dichloro-3′-(6-methoxy-5-((((5-oxopyrrolidin-2-yl)methyl)amino)methyl)pyridin-2-yl)[1,1′-biphenyl]-3- | |
| | yl)-2-(((2-fluoro-2-methylpropyl)amino)methyl)-3-methylpyrrolo[2,1-f][1,2,4]triazin-4(3H)-one | |
| 463 | 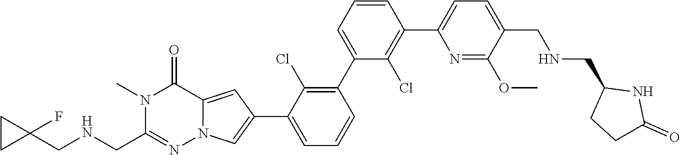 [SMILES: COc1nc(-c2cccc(-c3cccc(-c4cc5c(=O)n(C)c(CNCC6(F)CC6)nn5c4)c3Cl)c2Cl)ccc1CNC[C@@H]1CCC(=O)N1] | 0.036 |
| | (S)-6-(2,2′-dichloro-3′-(6-methoxy-5-((((5-oxopyrrolidin-2-yl)methyl)amino)methyl)pyridin-2-yl)-[1,1′-biphenyl]-3- | |
| | yl)-2-((((1-fluorocyclopropyl)methyl)amino)methyl)-3-methylpyrrolo[2,1-f][1,2,4]triazin-4(3H)-one | |
| 464 | 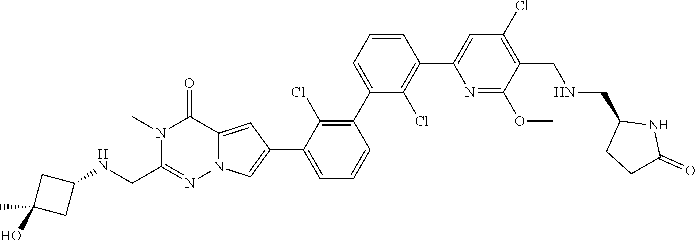 [SMILES: COc1nc(-c2cccc(-c3cccc(-c4cc5c(=O)n(C)c(CN[C@H]6C[C@](C)(O)C6)nn5c4)c3Cl)c2Cl)cc(Cl)c1CNC[C@@H]1CCC(=O)N1] | 0.0075 |
| | 6-(2,2′-dichloro-3′-(4-chloro-6-methoxy-5-(((((S)-5-oxopyrrolidin-2-yl)methyl)amino)methyl)pyridin-2-yl)-[1,1′- | |
| | biphenyl]-3-yl)-2-((((1r,3r)-3-hydroxy-3-methylcyclobutyl)amino)methyl)-3-methylpyrrolo[2,1-f][1,2,4]triazin- | |
| | 4(3H)-one | |
| 465 | 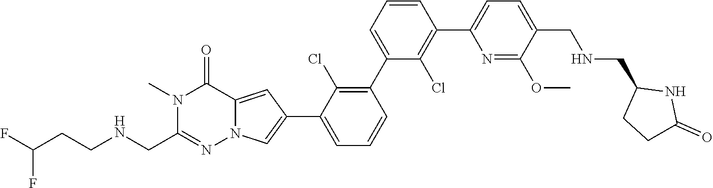 [SMILES: COc1nc(-c2cccc(-c3cccc(-c4cc5c(=O)n(C)c(CNCCC(F)F)nn5c4)c3Cl)c2Cl)ccc1CNC[C@@H]1CCC(=O)N1] | 0.014 |
| | (S)-6-(2,2′-dichloro-3′-(6-methoxy-5-((((5-oxopyrrolidin-2-yl)methyl)amino)methyl)pyridin-2-yl)-[1,1′-biphenyl]-3-yl)- | |
| | 2-(((3,3-difluoropropyl)amino)methyl)-3-methylpyrrolo[2,1-f][1,2,4]triazin-4(3H)-one | |
| 466 | 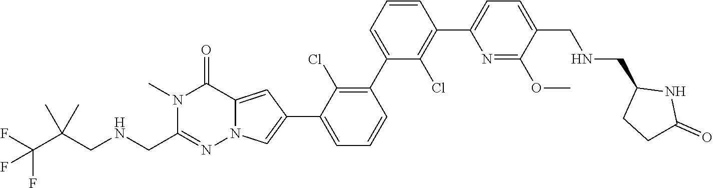 [SMILES: COc1nc(-c2cccc(-c3cccc(-c4cc5c(=O)n(C)c(CNCC(C)(C)C(F)(F)F)nn5c4)c3Cl)c2Cl)ccc1CNC[C@@H]1CCC(=O)N1] | 0.063 |
| | (S)-6-(2,2′-dichloro-3′-(6-methoxy-5-((((5-oxopyrrolidin-2-yl)methyl)amino)methyl)pyridin-2-yl)-[1,1′-biphenyl]-3-yl)-3- | |
| | methyl-2-(((3,3,3-trifluoro-2,2-dimethylpropyl)amino)methyl)pyrrolo[2,1-f][1,2,4]triazin-4(3H)-one | |
| 467 | 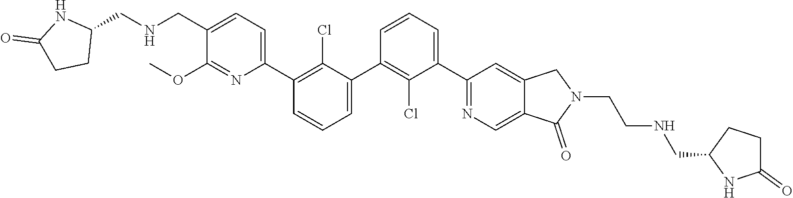 [SMILES: COc1nc(-c2cccc(-c3cccc(-c4cc5c(cn4)C(=O)N(CCNC[C@@H]4CCC(=O)N4)C5)c3Cl)c2Cl)ccc1CNC[C@@H]1CCC(=O)N1] | 0.0001 |
| | 6-(2,2′-dichloro-3′-(6-methoxy-5-(((((S)-5-oxopyrrolidin-2-yl)methyl)amino)methyl)pyridin-2-yl)-[1,1′-biphenyl]-3-yl)-2-(2-((((S)-5- | |
| | oxopyrrolidin-2-yl)methyl)amino)ethyl)-1,2-dihydro-3H-pyrrolo[3,4-c]pyridin-3-one | |
| 468 | 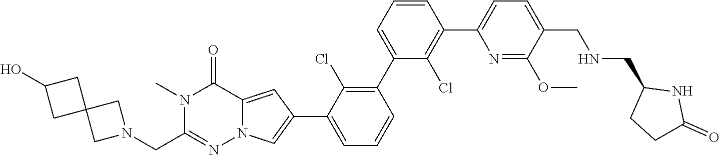 [SMILES: COc1nc(-c2cccc(-c3cccc(-c4cc5c(=O)n(C)c(CN6CC7(CC(O)C7)C6)nn5c4)c3Cl)c2Cl)ccc1CNC[C@@H]1CCC(=O)N1] | 0.0070 |
| | (S)-6-(2,2′-dichloro-3′-(6-methoxy-5-((((5-oxopyrrolidin-2-yl)methyl)amino)methyl)pyridin-2-yl)-[1,1′-biphenyl]-3-yl)-2- | |
| | ((6-hydroxy-2-azaspiro[3.3]heptan-2-yl)methyl)-3-methylpyrrolo[2,1-f][1,2,4]triazin-4(3H)-one | |
| 469 | 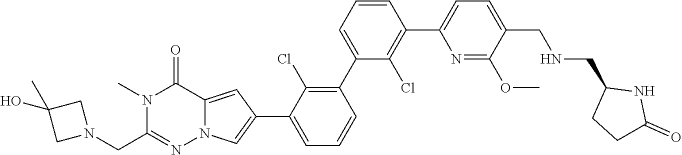 [SMILES: COc1nc(-c2cccc(-c3cccc(-c4cc5c(=O)n(C)c(CN6CC(C)(O)C6)nn5c4)c3Cl)c2Cl)ccc1CNC[C@@H]1CCC(=O)N1] | 0.0059 |
| | (S)-6-(2,2′-dichloro-3′-(6-methoxy-5-((((5-oxopyrrolidin-2-yl)methyl)amino)methyl)pyridin-2-yl)-[1,1′-biphenyl]-3- | |
| | yl)-2-((3-hydroxy-3-methylazetidin-1-yl)methyl)-3-methylpyrrolo[2,1-f][1,2,4]triazin-4(3H)-one | |
| 470 | 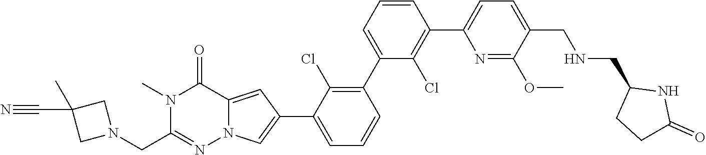 [SMILES: COc1nc(-c2cccc(-c3cccc(-c4cc5c(=O)n(C)c(CN6CC(C)(C#N)C6)nn5c4)c3Cl)c2Cl)ccc1CNC[C@@H]1CCC(=O)N1] | 0.037 |
| | (S)-1-((6-(2,2′-dichloro-3′-(6-methoxy-5-((((5-oxopyrrolidin-2-yl)methyl)amino)methyl)pyridin-2-yl)-[1,1′-biphenyl]-3- | |
| | yl)-3-methyl-4-oxo-3,4-dihydropyrrolo[2,1-f][1,2,4]triazin-2-yl)methyl)-3-methylazetidine-3-carbonitrile | |
| 471 | 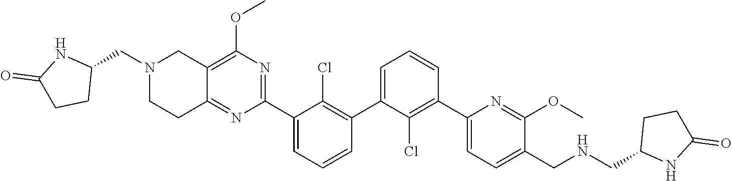 [SMILES: COc1nc(-c2cccc(-c3cccc(-c4nc5c(c(OC)n4)CN(C[C@@H]4CCC(=O)N4)CC5)c3Cl)c2Cl)ccc1CNC[C@@H]1CCC(=O)N1] | 0.0001 |
| | (S)-5-((((6-(2,2′-dichloro-3′-(4-methoxy-6-(((S)-5-oxopyrrolidin-2-yl)methyl)-5,6,7,8-tetrahydropyrido[4,3-d]pyrimidin-2- | |
| | yl)-[1,1′-biphenyl]-3-yl)-2-methoxypyridin-3-yl)methyl)amino)methyl)pyrrolidin-2-one | |
| 472 | 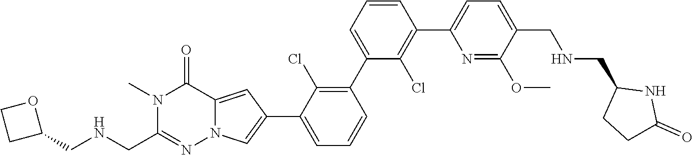 [SMILES: COc1nc(-c2cccc(-c3cccc(-c4cc5c(=O)n(C)c(CNC[C@@H]6CCO6)nn5c4)c3Cl)c2Cl)ccc1CNC[C@@H]1CCC(=O)N1] | 0.40 |
| | 6-(2,2′-dichloro-3′-(6-methoxy-5-(((((S)-5-oxopyrrolidin-2-yl)methyl)amino)methyl)pyridin-2-yl)-[1,1′-biphenyl]-3- | |
| | yl)-3-methyl-2-(((((S)-oxetan-2-yl)methyl)amino)methyl)pyrrolo[2,1-f][1,2,4]triazin-4(3H)-one | |
| 473 | 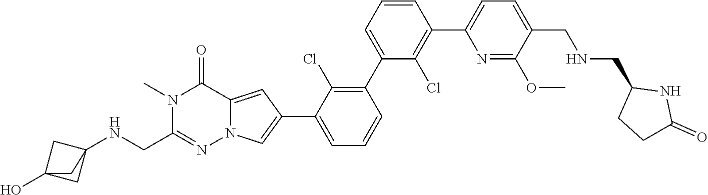 [SMILES: COc1nc(-c2cccc(-c3cccc(-c4cc5c(=O)n(C)c(CNC67CC(O)(C6)C7)nn5c4)c3Cl)c2Cl)ccc1CNC[C@@H]1CCC(=O)N1] | 0.0053 |
| | (S)-6-(2,2′-dichloro-3′-(6-methoxy-5-((((5-oxopyrrolidin-2-yl)methyl)amino)methyl)pyridin-2-yl)-[1,1′-biphenyl]-3-yl)- | |
| | 2-(((3-hydroxybicyclo[1.1.1]pentan-1-yl)amino)methyl)-3-methylpyrrolo[2,1-f][1,2,4]triazin-4(3H)-one | |
| 474 | 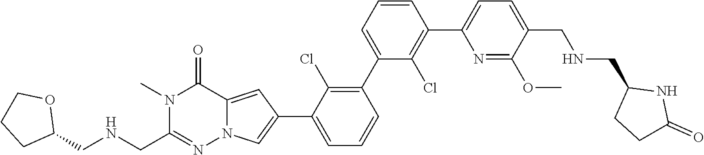 [SMILES: COc1nc(-c2cccc(-c3cccc(-c4cc5c(=O)n(C)c(CNC[C@@H]6CCCO6)nn5c4)c3Cl)c2Cl)ccc1CNC[C@@H]1CCC(=O)N1] | 0.021 |
| | 6-(2,2′-dichloro-3′-(6-methoxy-5-(((((S)-5-oxopyrrolidin-2-yl)methyl)amino)methyl)pyridin-2-yl)-[1,1′-biphenyl]-3-yl)- | |
| | 3-methyl-2-(((((S)-tetrahydrofuran-2-yl)methyl)amino)methyl)pyrrolo[2,1-f][1,2,4]triazin-4(3H)-one | |
| 475 | 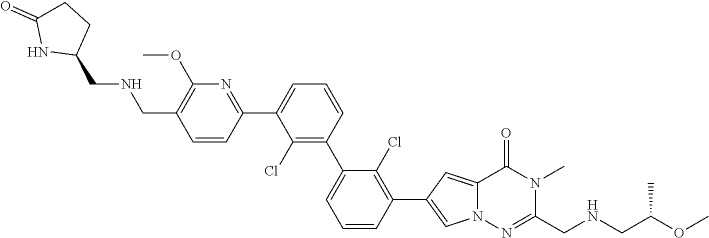 [SMILES: COc1nc(-c2cccc(-c3cccc(-c4cc5c(=O)n(C)c(CNC[C@H](C)OC)nn5c4)c3Cl)c2Cl)ccc1CNC[C@@H]1CCC(=O)N1] | 0.53 |
| | 6-(2,2′-dichloro-3′-(6-methoxy-5-(((((S)-5-oxopyrrolidin-2-yl)methyl)amino)methyl)pyridin-2-yl)-[1,1′-biphenyl]-3-yl)- | |
| | 2-((((S)-2-methoxypropyl)amino)methyl)-3-methylpyrrolo[2,1-f][1,2,4]triazin-4(3H)-one | |
| 476 | 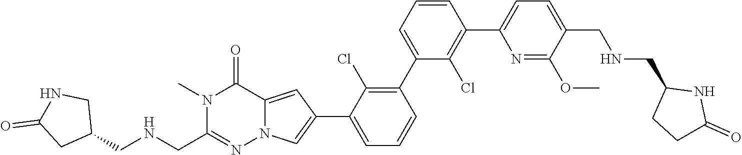 [SMILES: COc1nc(-c2cccc(-c3cccc(-c4cc5c(=O)n(C)c(CNC[C@H]6CNC(=O)C6)nn5c4)c3Cl)c2Cl)ccc1CNC[C@@H]1CCC(=O)N1] | 0.033 |
| | 6-(2,2′-dichloro-3′-(6-methoxy-5-(((((S)-5-oxopyrrolidin-2-yl)methyl)amino)methyl)pyridin-2-yl)-[1,1′-biphenyl]-3-yl)-3- | |
| | methyl-2-(((((S)-5-oxopyrrolidin-3-yl)methyl)amino)methyl)pyrrolo[2,1-f][1,2,4]triazin-4(3H)-one | |
| 477 | 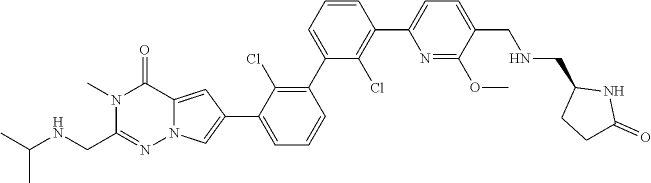 [SMILES: COc1nc(-c2cccc(-c3cccc(-c4cc5c(=O)n(C)c(CNC(C)C)nn5c4)c3Cl)c2Cl)ccc1CNC[C@@H]1CCC(=O)N1] | 0.017 |
| | (S)-6-(2,2′-dichloro-3′-(6-methoxy-5-((((5-oxopyrrolidin-2-yl)methyl)amino)methyl)pyridin-2-yl)-[1,1′- | |
| | biphenyl]-3-yl)-2-((isopropylamino)methyl)-3-methylpyrrolo[2,1-f][1,2,4]triazin-4(3H)-one | |
| 478 | 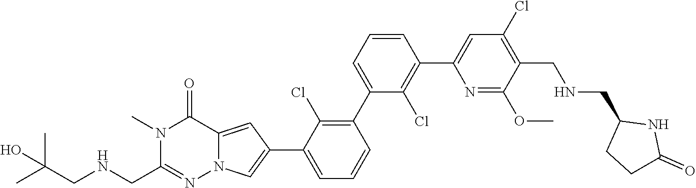 [SMILES: COc1nc(-c2cccc(-c3cccc(-c4cc5c(=O)n(C)c(CNCC(C)(C)O)nn5c4)c3Cl)c2Cl)cc(Cl)c1CNC[C@@H]1CCC(=O)N1] | 0.029 |
| | (S)-6-(2,2′-dichloro-3′-(4-chloro-6-methoxy-5-((((5-oxopyrrolidin-2-yl)methyl)amino)methyl)pyridin-2-yl)-[1,1′- | |
| | biphenyl]-3-yl)-2-(((2-hydroxy-2-methylpropyl)amino)methyl-3-methylpyrrolo[2,1-f][1,2,4]triazin-4(3H)-one | |
| 479 | 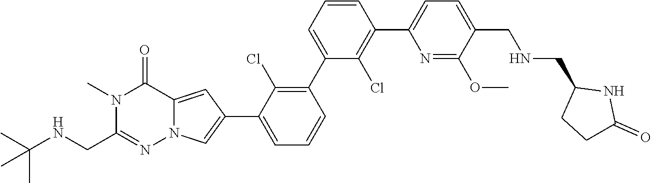 [SMILES: COc1nc(-c2cccc(-c3cccc(-c4cc5c(=O)n(C)c(CNC(C)(C)C)nn5c4)c3Cl)c2Cl)ccc1CNC[C@@H]1CCC(=O)N1] | 0.014 |
| | (S)-2-((tert-butylamino)methyl)-6-(2,2′-dichloro-3′-(6-methoxy-5-((((5-oxopyrrolidin-2- | |
| | yl)methyl)amino)methyl)pyridin-2-yl)-[1,1′-biphenyl]-3-yl)-3-methylpyrrolo[2,1-f][1,2,4]triazin-4(3H)-one | |
| 480 | 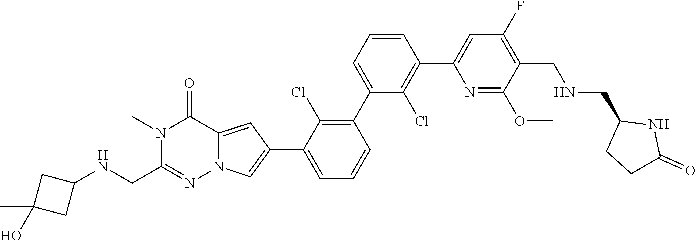 [SMILES: COc1nc(-c2cccc(-c3cccc(-c4cc5c(=O)n(C)c(CNC6CC(C)(O)C6)nn5c4)c3Cl)c2Cl)cc(F)c1CNC[C@@H]1CCC(=O)N1] | 0.069 |
| | (S)-6-(2,2′-dichloro-3′-(4-fluoro-6-methoxy-5-((((5-oxopyrrolidin-2-yl)methyl)amino)methyl)pyridin-2-yl)-[1,1′- | |
| | biphenyl]-3-yl)-2-(((3-hydroxy-3-methylcyclobutyl)amino)methyl)-3-methylpyrrolo[2,1-f][1,2,4]triazin-4(3H)-one | |
| 481 | 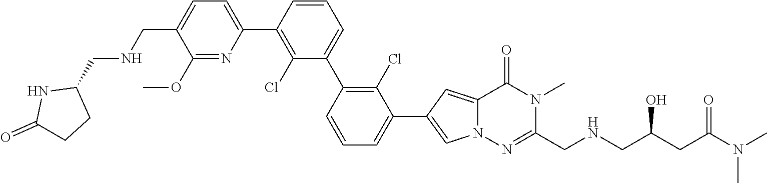 [SMILES: COc1nc(-c2cccc(-c3cccc(-c4cc5c(=O)n(C)c(CNC[C@@H](O)CC(=O)N(C)C)nn5c4)c3Cl)c2Cl)ccc1CNC[C@@H]1CCC(=O)N1] | 0.042 |
| | (S)-4-(((6-(2,2′-dichloro-3′-(6-methoxy-5-(((((S)-5-oxopyrrolidin-2-yl)methyl)amino)methyl)pyridin-2-yl)-[1,1′-biphenyl]-3-yl)-3- | |
| | methyl-4-oxo-3,4-dihydropyrrolo[2,1-f][1,2,4]triazin-2-yl)methyl)amino)-3-hydroxy-N,N-dimethylbutanamide | |
| 482 | 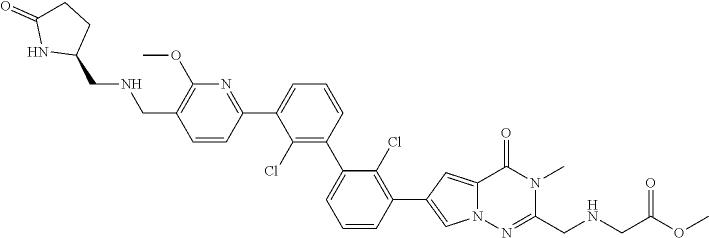 [SMILES: COC(=O)CNCc1nn2cc(-c3cccc(-c4cccc(-c5ccc(CNC[C@@H]6CCC(=O)N6)c(OC)n5)c4Cl)c3Cl)cc2c(=O)n1C] | 0.028 |
| | methyl (S)-((6-(2,2′-dichloro-3′-(6-methoxy-5-((((5-oxopyrrolidin-2-yl)methyl)amino)methyl)pyridin-2-yl)-[1,1′- | |
| | biphenyl]-3-yl)-3-methyl-4-oxo-3,4-dihydropyrrolo[2,1-f][1,2,4]triazin-2-yl)methyl)glycinate | |
| 483 | 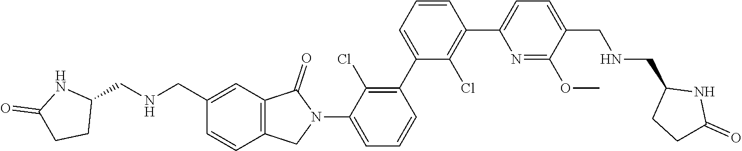 [SMILES: COc1nc(-c2cccc(-c3cccc(N4Cc5ccc(CNC[C@@H]6CCC(=O)N6)cc5C4=O)c3Cl)c2Cl)ccc1CNC[C@@H]1CCC(=O)N1] | 0.0001 |
| | 2-(2,2′-dichloro-3′-(6-methoxy-5-(((((S)-5-oxopyrrolidin-2-yl)methyl)amino)methyl)pyridin-2-yl)-[1,1′-biphenyl]-3-yl)-6- | |
| | (((((S)-5-oxopyrrolidin-2-yl)methyl)amino)methyl)isoindolin-1-one | |
| 484 | 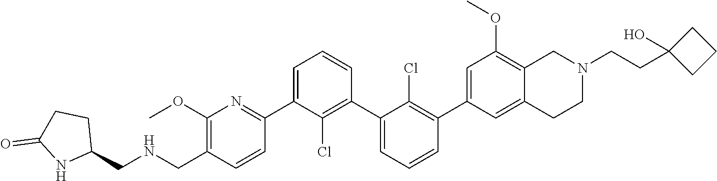 [SMILES: COc1cc(-c2cccc(-c3cccc(-c4ccc(CNC[C@@H]5CCC(=O)N5)c(OC)n4)c3Cl)c2Cl)cc2c1CN(CCC1(O)CCC1)CC2] | 0.0003 |
| | (S)-5-((((6-(2,2′-dichloro-3′-(2-(2-(1-hydroxycyclobutyl)ethyl)-8-methoxy-1,2,3,4-tetrahydroisoquinolin-6-yl)-[1,1′- | |
| | biphenyl]-3-yl)-2-methoxypyridin-3-yl)methyl)amino)methyl)pyrrolidin-2-one | |
| 485 | 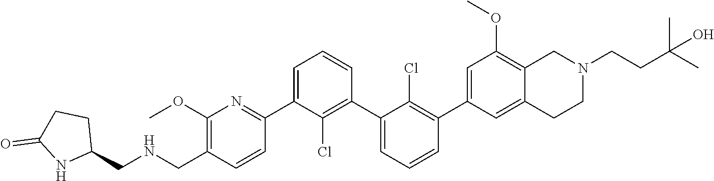 [SMILES: COc1cc(-c2cccc(-c3cccc(-c4ccc(CNC[C@@H]5CCC(=O)N5)c(OC)n4)c3Cl)c2Cl)cc2c1CN(CCC(C)(C)O)CC2] | 0.0001 |
| | (S)-5-((((6-(2,2′-dichloro-3′-(2-(3-hydroxy-3-methylbutyl)-8-methoxy-1,2,3,4-tetrahydroisoquinolin-6-yl)-[1,1′-biphenyl]- | |
| | 3-yl)-2-methoxypyridin-3-yl)methyl)amino)methyl)pyrrolidin-2-one | |
| 486 | 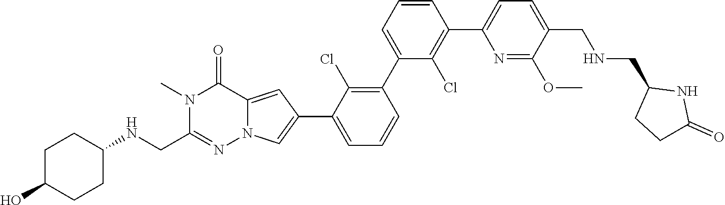 [SMILES: COc1nc(-c2cccc(-c3cccc(-c4cc5c(=O)n(C)c(CN[C@H]6CC[C@H](O)CC6)nn5c4)c3Cl)c2Cl)ccc1CNC[C@@H]1CCC(=O)N1] | 0.0004 |
| | 6-(2,2′-dichloro-3′-(6-methoxy-5-(((((S)-5-oxopyrrolidin-2-yl)methyl)amino)methyl)pyridin-2-yl)-[1,1′-biphenyl]-3-yl)-2- | |
| | ((((1r,4r)-4-hydroxycyclohexyl)amino)methyl)-3-methylpyrrolo[2,1-f][1,2,4]triazin-4(3H)-one | |
| 487 | 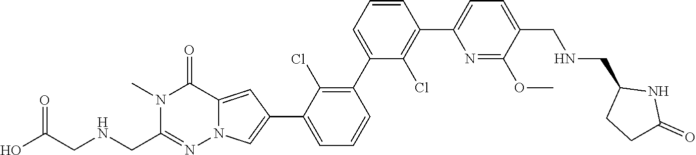 [SMILES: COc1nc(-c2cccc(-c3cccc(-c4cc5c(=O)n(C)c(CNCC(=O)O)nn5c4)c3Cl)c2Cl)ccc1CNC[C@@H]1CCC(=O)N1] | 0.0001 |
| | (S)-((6-(2,2′-dichloro-3′-(6-methoxy-5-((((5-oxopyrrolidin-2-yl)methyl)amino)methyl)pyridin-2-yl)-[1,1′-biphenyl]-3- | |
| | yl)-3-methyl-4-oxo-3,4-dihydropyrrolo[2,1-f][1,2,4]triazin-2-yl)methyl)glycine | |
| 488 | 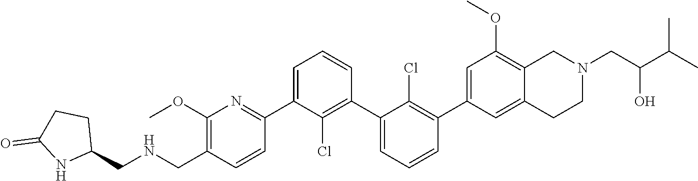 [SMILES: COc1cc(-c2cccc(-c3cccc(-c4ccc(CNC[C@@H]5CCC(=O)N5)c(OC)n4)c3Cl)c2Cl)cc2c1CN(CC(O)C(C)C)CC2] | 0.0002 |
| | (5S)-5-((((6-(2,2′-dichloro-3′-(2-(2-hydroxy-3-methylbutyl)-8-methoxy-1,2,3,4-tetrahydroisoquinolin-6-yl)-[1,1′- | |
| | biphenyl]-3-yl)-2-methoxypyridin-3-yl)methyl)amino)methyl)pyrrolidin-2-one | |
| | mixture of 2 diastereoisomers | |
| 489 | 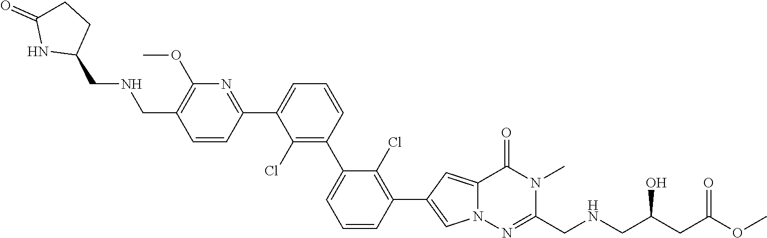 [SMILES: COC(=O)C[C@H](O)CNCc1nn2cc(-c3cccc(-c4cccc(-c5ccc(CNC[C@@H]6CCC(=O)N6)c(OC)n5)c4Cl)c3Cl)cc2c(=O)n1C] | 1.5 |
| | methyl (S)-4-(((6-(2,2′-dichloro-3′-(6-methoxy-5-(((((S)-5-oxopyrrolidin-2-yl)methyl)amino)methyl)pyridin-2-yl)-[1,1′-biphenyl]- | |
| | 3-yl)-3-methyl-4-oxo-3,4-dihydropyrrolo[2,1-f][1,2,4]triazin-2-yl)methyl)amino)-3-hydroxybutanoate | |
| 490 | 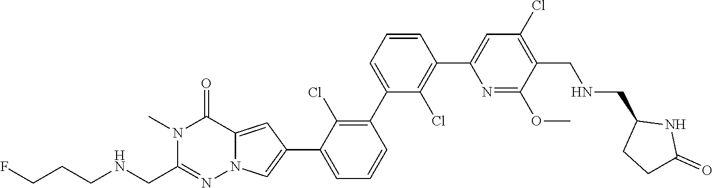 [SMILES: COc1nc(-c2cccc(-c3cccc(-c4cc5c(=O)n(C)c(CNCCCF)nn5c4)c3Cl)c2Cl)cc(Cl)c1CNC[C@@H]1CCC(=O)N1] | 0.0001 |
| | (S)-6-(2,2′-dichloro-3′-(4-chloro-6-methoxy-5-((((5-oxopyrrolidin-2-yl)methyl)amino)methyl)pyridin-2-yl)-[1,1′- | |
| | biphenyl]-3-yl)-2-(((3-fluoropropyl)amino)methyl)-3-methylpyrrolo[2,1-f][1,2,4]triazin-4(3H)-one | |
| 491 | 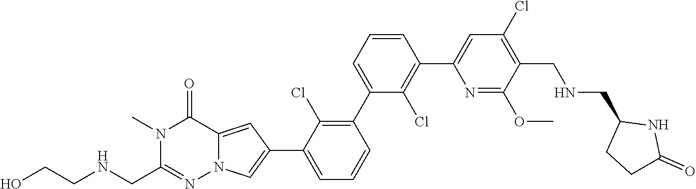 [SMILES: COc1nc(-c2cccc(-c3cccc(-c4cc5c(=O)n(C)c(CNCCO)nn5c4)c3Cl)c2Cl)cc(Cl)c1CNC[C@@H]1CCC(=O)N1] | 0.0007 |
| | (S)-6-(2,2′-dichloro-3′-(4-chloro-6-methoxy-5-((((5-oxopyrrolidin-2-yl)methyl)amino)methyl)pyridin-2-yl)-[1,1′- | |
| | biphenyl]-3-yl)-2-(((2-hydroxyethyl)amino)methyl)-3-methylpyrrolo[2,1-f][1,2,4]triazin-4(3H)-one | |
| 492 | 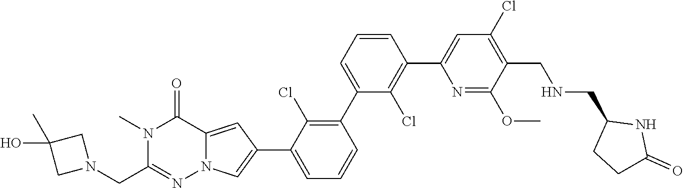 [SMILES: COc1nc(-c2cccc(-c3cccc(-c4cc5c(=O)n(C)c(CN6CC(C)(O)C6)nn5c4)c3Cl)c2Cl)cc(Cl)c1CNC[C@@H]1CCC(=O)N1] | 0.0006 |
| | (S)-6-(2,2′-dichloro-3′-(4-chloro-6-methoxy-5-((((5-oxopyrrolidin-2-yl)methyl)amino)methyl)pyridin-2-yl)-[1,1′- | |
| | biphenyl]-3-yl)-2-((3-hydroxy-3-methylazetidin-1-yl)methyl)-3-methylpyrrolo[2,1-f][1,2,4]triazin-4(3H)-one | |
| 493 | 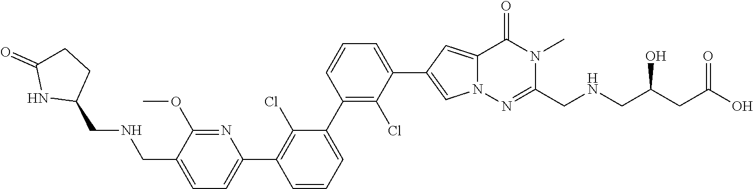 [SMILES: COc1nc(-c2cccc(-c3cccc(-c4cc5c(=O)n(C)c(CNC[C@@H](O)CC(=O)O)nn5c4)c3Cl)c2Cl)ccc1CNC[C@@H]1CCC(=O)N1] | 0.0001 |
| | (S)-4-(((6-(2,2′-dichloro-3′-(6-methoxy-5-(((((S)-5-oxopyrrolidin-2-yl)methyl)amino)methyl)pyridin-2-yl)-[1,1′-biphenyl]-3-yl)- | |
| | 3-methyl-4-oxo-3,4-dihydropyrrolo[2,1-f][1,2,4]triazin-2-yl)methyl)amino)-3-hydroxybutanoic acid | |
| 494 | 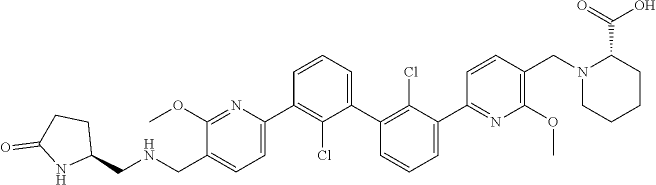 [SMILES: COc1nc(-c2cccc(-c3cccc(-c4ccc(CN5CCCC[C@H]5C(=O)O)c(OC)n4)c3Cl)c2Cl)ccc1CNC[C@@H]1CCC(=O)N1] | 0.0001 |
| | (S)-1-((6-(2,2′-dichloro-3′-(6-methoxy-5-(((((S)-5-oxopyrrolidin-2-yl)methyl)amino)methyl)pyridin-2-yl)-[1,1′- | |
| | biphenyl]-3-yl)-2-methoxypyridin-3-yl)methyl)piperidine-2-carboxylic acid | |
| 495 | 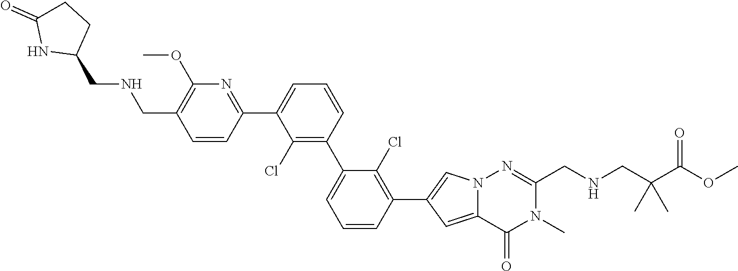 [SMILES: COC(=O)C(C)(C)CNCc1nn2cc(-c3cccc(-c4cccc(-c5ccc(CNC[C@@H]6CCC(=O)N6)c(OC)n5)c4Cl)c3Cl)cc2c(=O)n1C] | 0.0007 |
| | methyl (S)-3-(((6-(2,2′-dichloro-3′-(6-methoxy-5-((((5-oxopyrrolidin-2-yl)methyl)amino)methyl)pyridin-2-yl)-[1,1′-biphenyl]- | |
| | 3-yl)-3-methyl-4-oxo-3,4-dihydropyrrolo[2,1-f][1,2,4]triazin-2-yl)methyl)amino)-2,2-dimethylpropanoate | |
| 496 | 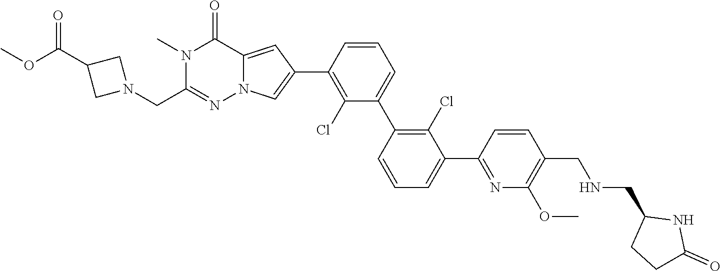 [SMILES: COC(=O)C1CN(Cc2nn3cc(-c4cccc(-c5cccc(-c6ccc(CNC[C@@H]7CCC(=O)N7)c(OC)n6)c5Cl)c4Cl)cc3c(=O)n2C)C1] | |
| | methyl (S)-1-((6-(2,2′-dichloro-3′-(6-methoxy-5-((((5-oxopyrrolidin-2-yl)methyl)amino)methyl)pyridin-2-yl)-[1,1′- | |
| | biphenyl]-3-yl)-3-methyl-4-oxo-3,4-dihydropyrrolo[2,1-f][1,2,4]triazin-2-yl)methyl)azetidine-3-carboxylate | |

The disclosures of each and every patent, patent application, and publication cited herein are hereby incorporated herein by reference in their entirety. While this invention has been disclosed with reference to specific embodiments, it is apparent that other embodiments and variations of this invention may be devised by others skilled in the art without departing from the true spirit and scope of the invention. The appended claims are intended to be construed to include all such embodiments and equivalent variations.

1. A compound, or a salt, solvate, geometric isomer, stereoisomer, or tautomer thereof, selected from the group consisting of:
(a) a compound of formula (Ib): 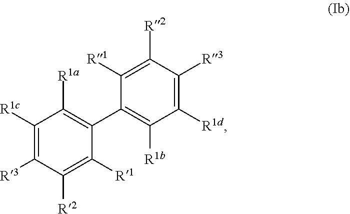
wherein:
R^{1a} is selected from the group consisting of H, C₁-C₃ alkyl, C₃-C₈ cycloalkyl, C₁-C₃ alkoxy, cyano, halogen, C₁-C₃ haloalkyl, and C₁-C₃ haloalkoxy;
R^{1b} is selected from the group consisting of H, C₁-C₃ alkyl, C₃-C₈ cycloalkyl, C₁-C₃ alkoxy, cyano, halogen, C₁-C₃ haloalkyl, and C₁-C₃ haloalkoxy;
R^{1c} is selected from the group consisting of: 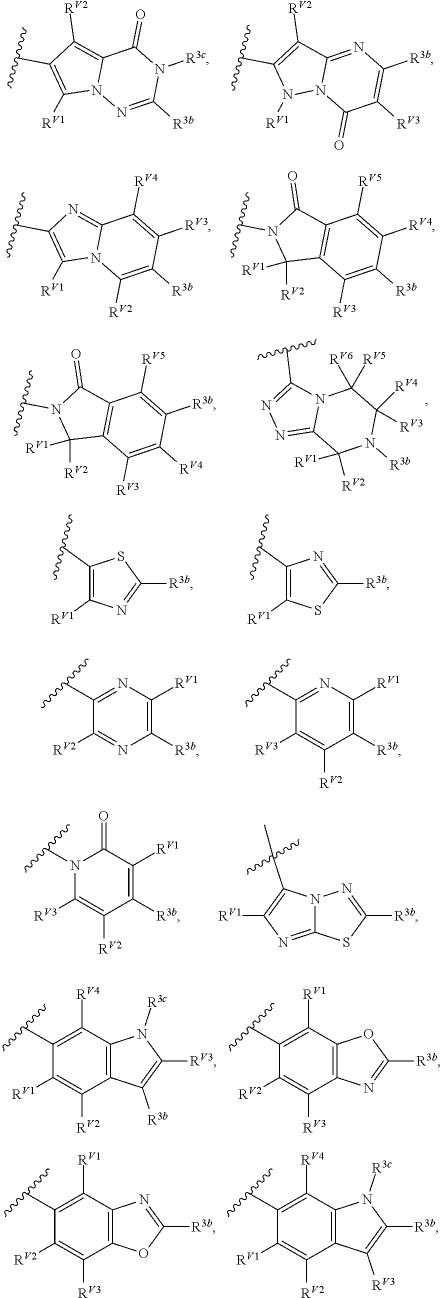 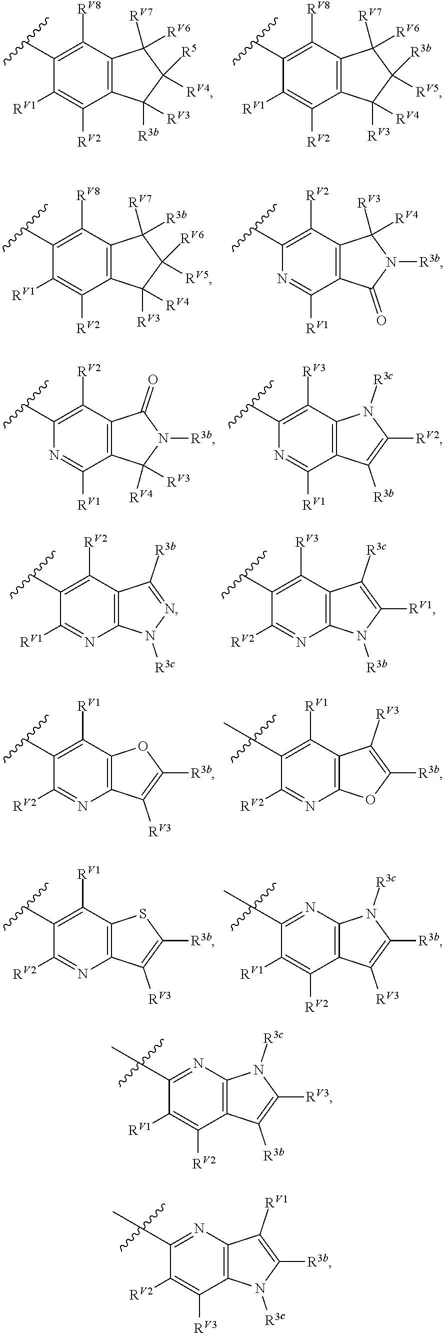 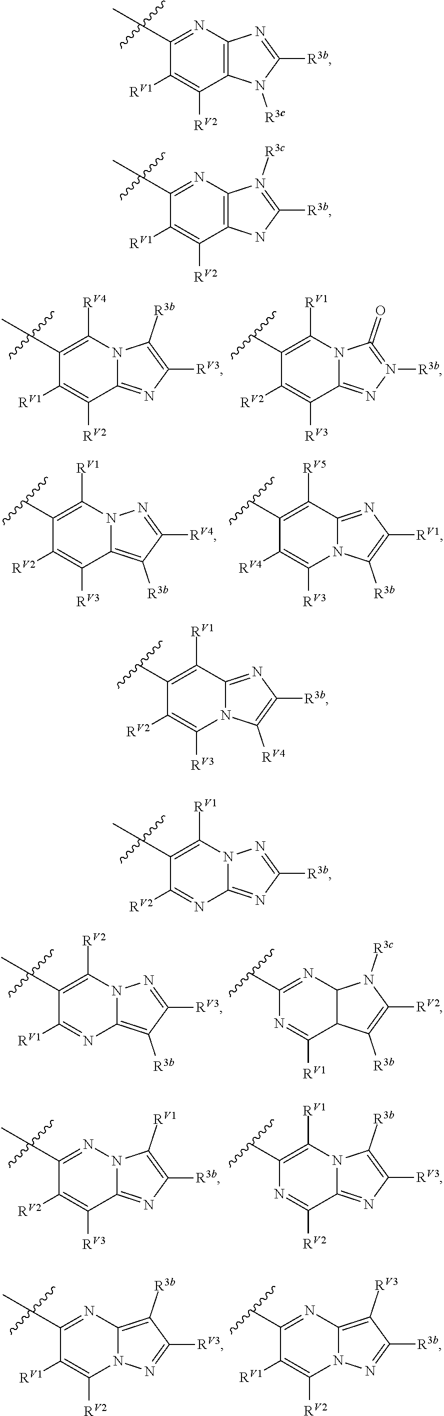 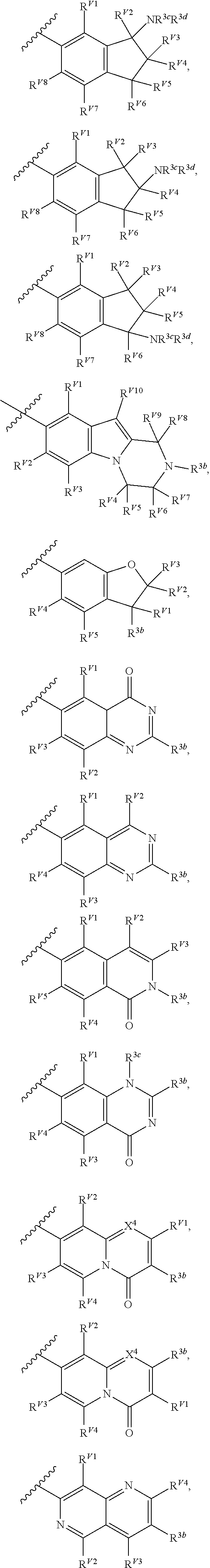 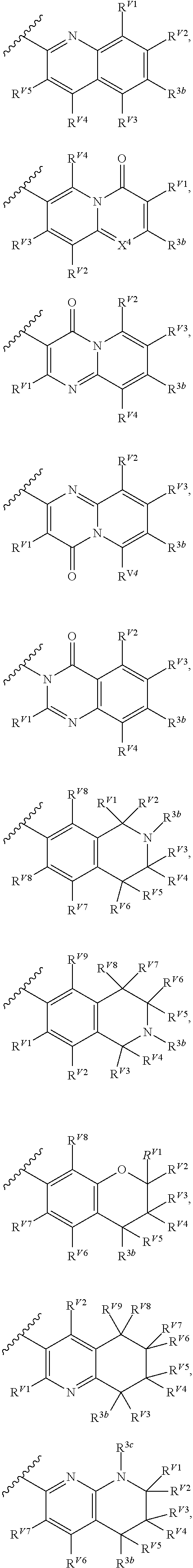 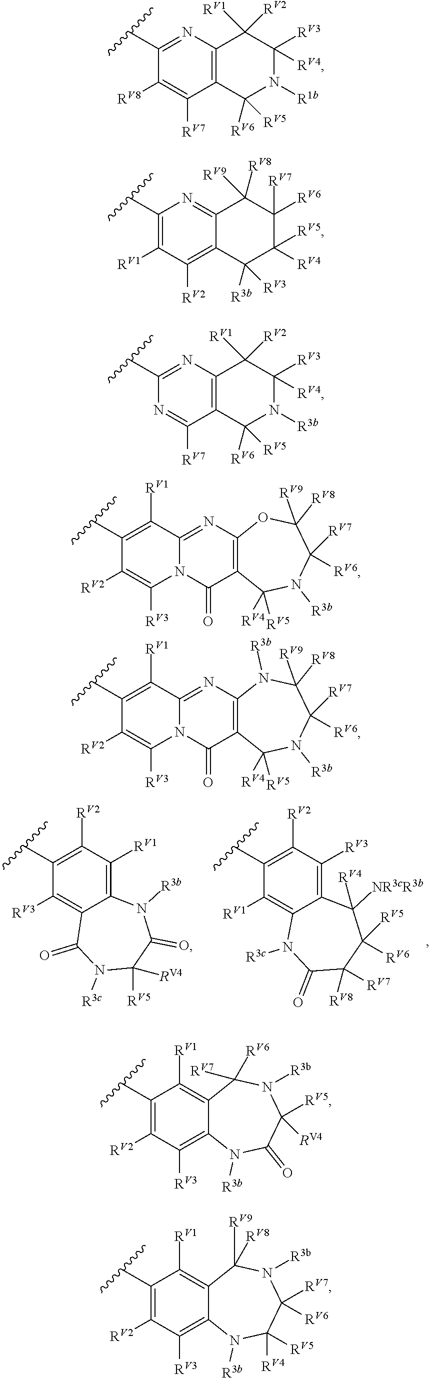 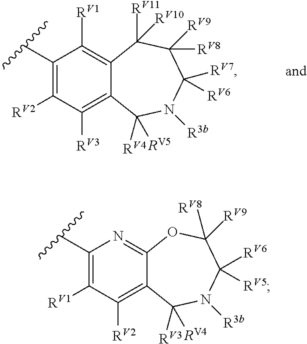
R^{1d} is selected from the group consisting of: 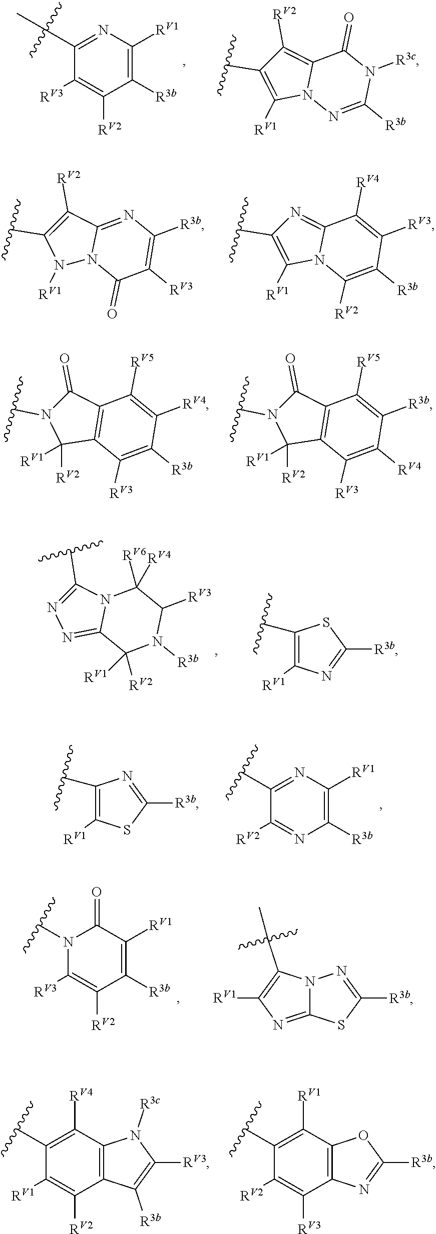 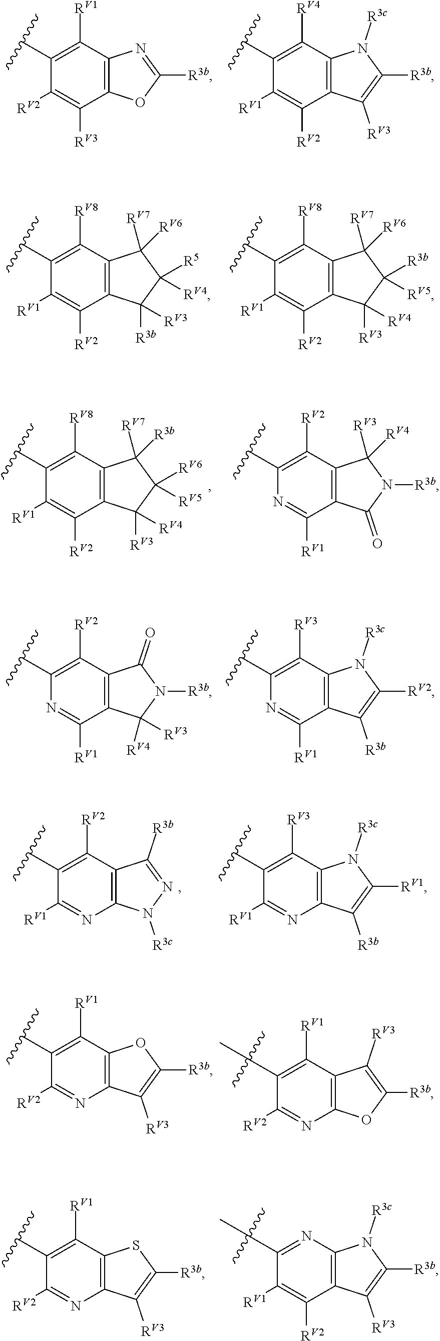 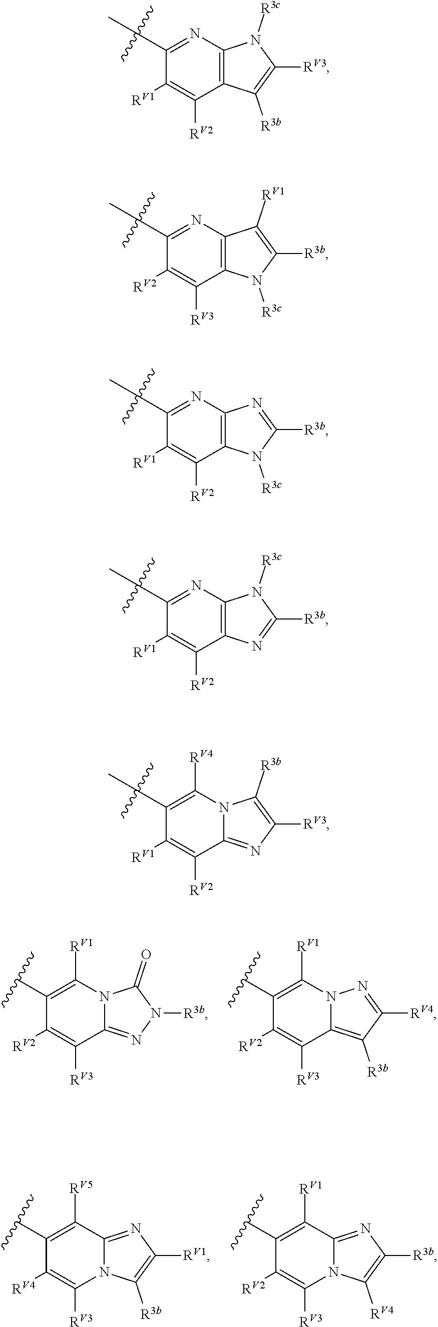 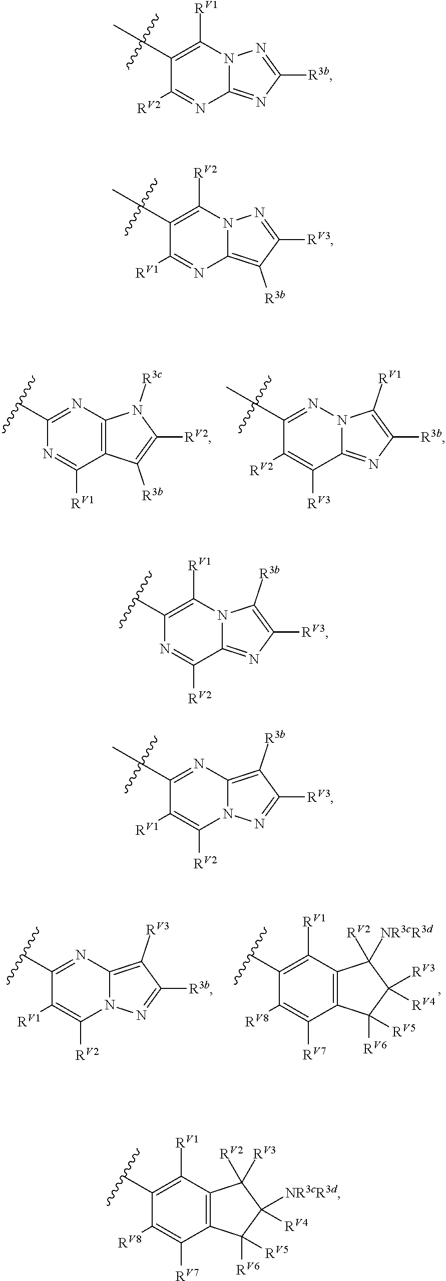 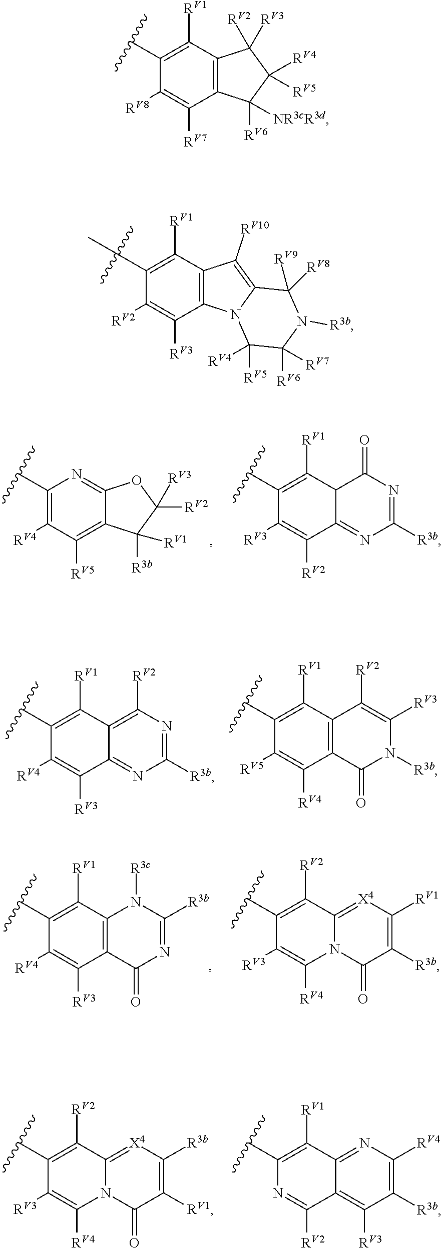 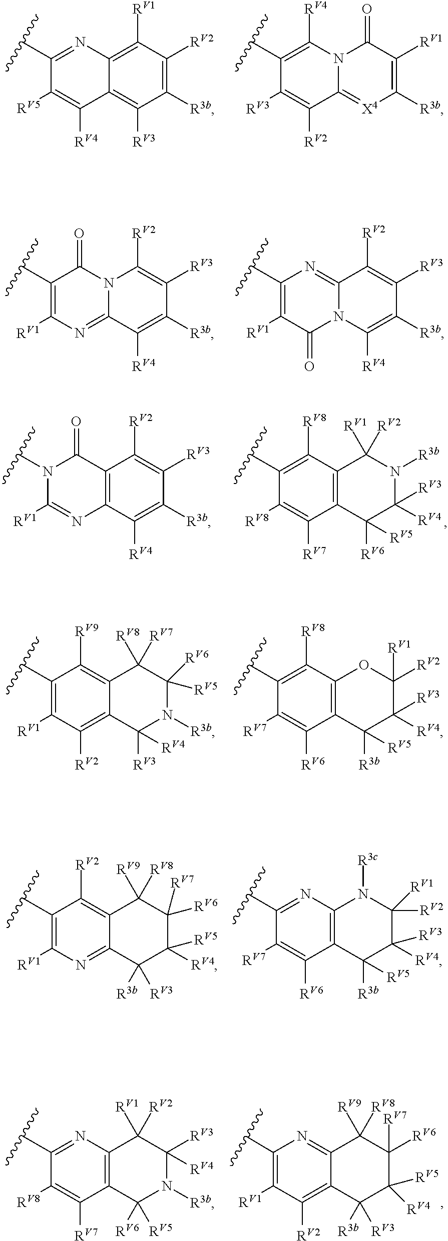 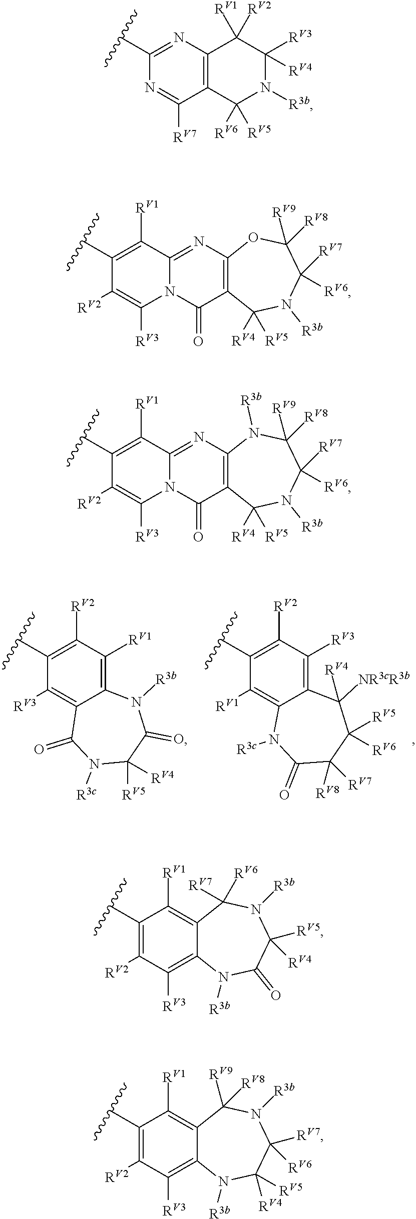 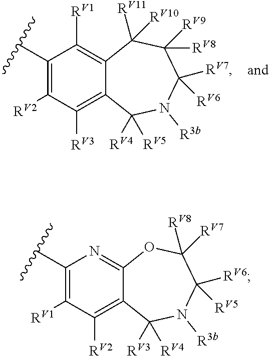
R′¹, R′², and R′³ are independently selected from the group consisting of H, C₁-C₃ alkyl, C₃-C₈ cycloalkyl, C₁-C₃ alkoxy, cyano, halogen, C₁-C₃ haloalkyl, and C₁-C₃ haloalkoxy;
R″¹, R″², and R″³ are independently selected from the group consisting of H, C₁-C₃ alkyl, C₃-C₈ cycloalkyl, C₁-C₃ alkoxy, cyano, halogen, C₁-C₃ haloalkyl, and C₁-C₃ haloalkoxy;
each occurrence of X⁴ is independently selected from the group consisting of CR^{X4} and N, wherein each occurrence of R^{X4} is selected from the group consisting of H, C₁-C₃ alkyl, C₃-C₈ cycloalkyl, C₁-C₃ alkoxy, cyano, halogen, C₁-C₃ haloalkyl, and C₁-C₃ haloalkoxy;
R^{3a} is selected from the group consisting of —CHO, —C(O)OR^{III}, —C(═O)NR^{III}R^{III}, —C(═NR^{S})NR^{III}R^{III}, optionally substituted heterocyclyl, —(CH₂)₁₋₃(optionally substituted heterocyclyl), optionally substituted C₁-C₆ alkoxy, optionally substituted C₁-C₆ aminoalkyl, and optionally substituted C₁-C₆ hydroxyalkyl,
wherein each occurrence of R^{III} is independently H, C₁-C₆ alkyl, or C₃-C₈ cycloalkyl, wherein the alkyl or cycloalkyl is independently optionally substituted with at least one of halogen, —OH, C₁-C₆ alkoxy, —NH₂, —NH(C₁-C₆ alkyl), and —N(C₁-C₆ alkyl)(C₁-C₆ alkyl),
or two R^{III} can combine with the N atom to which they are bound to form 3-membered to 8-membered optionally substituted heterocyclyl,
or, if R^{3a} is —C(═NR⁵)NR^{III}R^{III}, then R⁵ and one Rm can combine to form 4-membered to 8-membered optionally substituted heterocyclyl,
or, if X³ is CR^{X3} and R^{3a} has a tetravalent a carbon, then optionally RX³ and the tetravalent a carbon of R^{3a} combine to form a 3-membered to to 8-membered carbocyclyl;
each occurrence of R^{3b} is independently selected from the group consisting of H, optionally substituted C₁-C₆ alkyl, optionally substituted C₁-C₆ alkoxy, optionally substituted C₁-C₆ aminoalkyl, optionally substituted C₁-C₆ hydroxyalkyl, optionally substituted C₃-C₈ cycloalkyl, —CHO, —C(O)OR^{IV}, —C(═O)NR^{IV}R^{IV}, —C(═NR⁵)NR^{IV}R^{IV}, —NR^{3d}(optionally substituted alkyl), optionally substituted heterocyclyl, and —(CH₂)₁₋₃(optionally substituted heterocyclyl),
wherein each occurrence of R^{IV} is independently H, C₁-C₆ alkyl, or C₃-C₈ cycloalkyl, wherein the alkyl or cycloalkyl is independently optionally substituted with at least one of halogen, —OH, C₁-C₆ alkoxy, —NH₂, —NH(C₁-C₆ alkyl), and —N(C₁-C₆ alkyl)(C₁-C₆ alkyl),
or two R^{IV} can combine with the N atom to which they are bound to form 3-membered to 8-membered optionally substituted heterocyclyl,
or, if R^{3b} is —C(═NR⁵)NR^{IV}R^{IV}, then R⁵ and one R^{IV} can combine to form 4-membered to 8-membered optionally substituted heterocyclyl;
each occurrence of R³, is independently selected from the group consisting of H, optionally substituted C₁-C₆ alkyl, optionally substituted C₃-C₈ cycloalkyl, and optionally substituted C₁-C₆ acyl;
each occurrence of R^{3d} is independently selected from the group consisting of H, optionally substituted C₁-C₆ alkyl, optionally substituted C₃-C₈ cycloalkyl, and optionally substituted C₁-C₆ acyl;
each occurrence of R⁵ is independently H, C₁-C₆ alkyl, or C₃-C₈ cycloalkyl, wherein the alkyl or cycloalkyl is independently optionally substituted with at least one of halogen, —OH, C₁-C₆ alkoxy, —NH₂, —NH(C₁-C₆ alkyl), and —N(C₁-C₆ alkyl)(C₁-C₆ alkyl),
each occurrence of R^{V1}, R^{V2}, R^{V3}, R^{V4}, R^{V5}, R^{V6}, R^{V7}, R^{V8}, R^{V9}, RV¹⁰, and R^{V11} is independently selected from the group consisting of H, C₁-C₃ alkyl, C₃-C₈ cycloalkyl, C₁-C₃ alkoxy, cyano, halogen, C₁-C₃ haloalkyl, and C₁-C₃ haloalkoxy;
(b) a compound of formula (Ia): 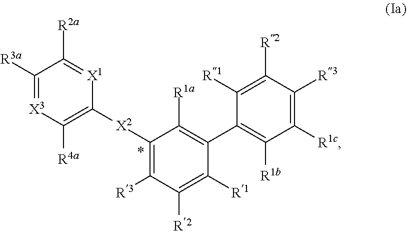
wherein:
X¹ is selected from the group consisting of CR^{X1} and N, wherein each occurrence of R^{X1} is independently selected from the group consisting of H, C₁-C₃ alkyl, C₃-C₈ cycloalkyl, C₁-C₃ alkoxy, cyano, halogen, C₁-C₃ haloalkyl, and C₁-C₃ haloalkoxy;
X² is selected from the group consisting of bond, —OCH₂—**, —CH₂O—**, —CH═CH—**, —CF═CH—**, —CH═CF—**, —CF═CF—**, —C(═O)NH—**, and —NHC(═O)—**,
wherein the bond marked with ** is to the phenyl ring carbon marked with *; 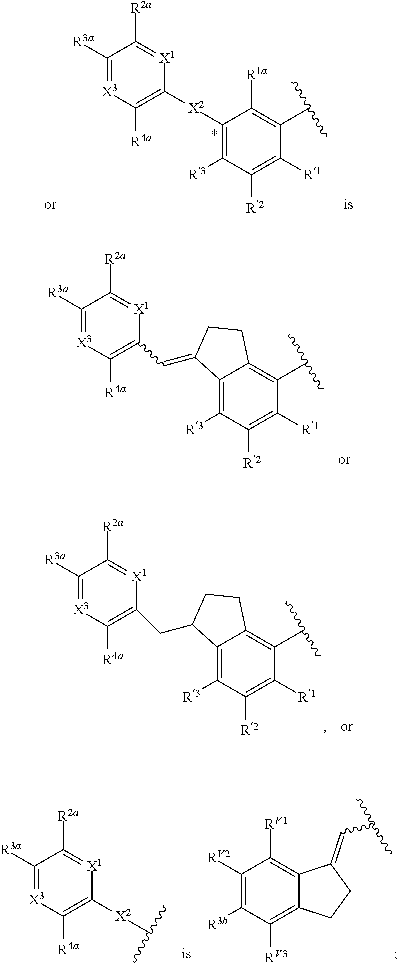
X³ is selected from the group consisting of CR^{X3} and N, wherein each occurrence of R^{X3} is independently selected from the group consisting of H, C₁-C₃ alkyl, C₃-C₈ cycloalkyl, C₁-C₃ alkoxy, cyano, halogen, C₁-C₃ haloalkyl, and C₁-C₃ haloalkoxy;
R′¹, R′², and R′³ are independently selected from the group consisting of H, C₁-C₃ alkyl, C₃-C₈ cycloalkyl, C₁-C₃ alkoxy, cyano, halogen, C₁-C₃ haloalkyl, and C₁-C₃ haloalkoxy;
R″1¹, R″², and R″³ are independently selected from the group consisting of H, C₁-C₃ alkyl, C₃-C₈ cycloalkyl, C₁-C₃ alkoxy, cyano, halogen, C₁-C₃ haloalkyl, and C₁-C₃ haloalkoxy;
R^{1a} is selected from the group consisting of H, C₁-C₃ alkyl, C₃-C₈ cycloalkyl, C₁-C₃ alkoxy, cyano, halogen, C₁-C₃ haloalkyl, and C₁-C₃ haloalkoxy;
R^{1b} is selected from the group consisting of H, C₁-C₃ alkyl, C₃-C₈ cycloalkyl, C₁-C₃ alkoxy, cyano, halogen, C₁-C₃ haloalkyl, and C₁-C₃ haloalkoxy;
R^{1c} is selected from the group consisting of: 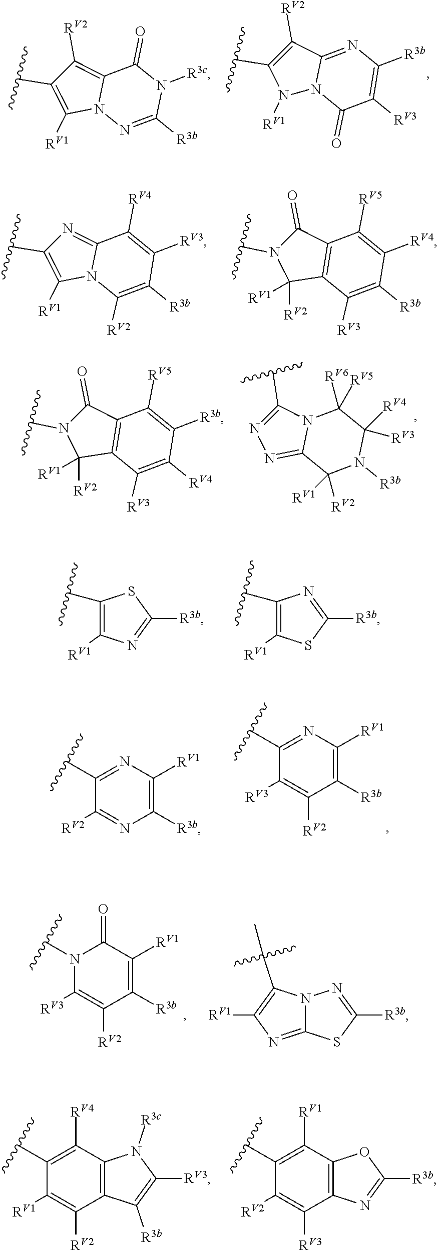 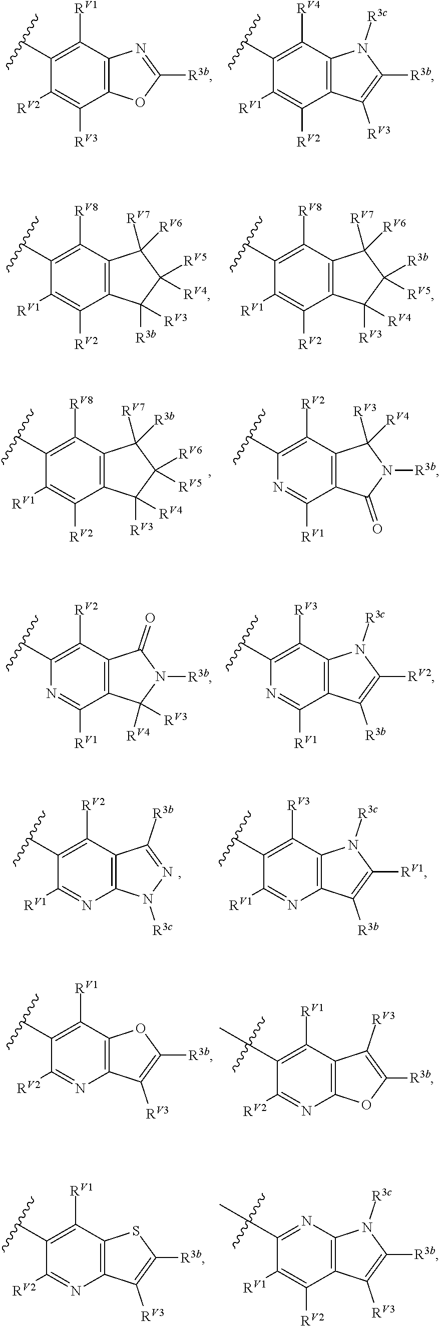 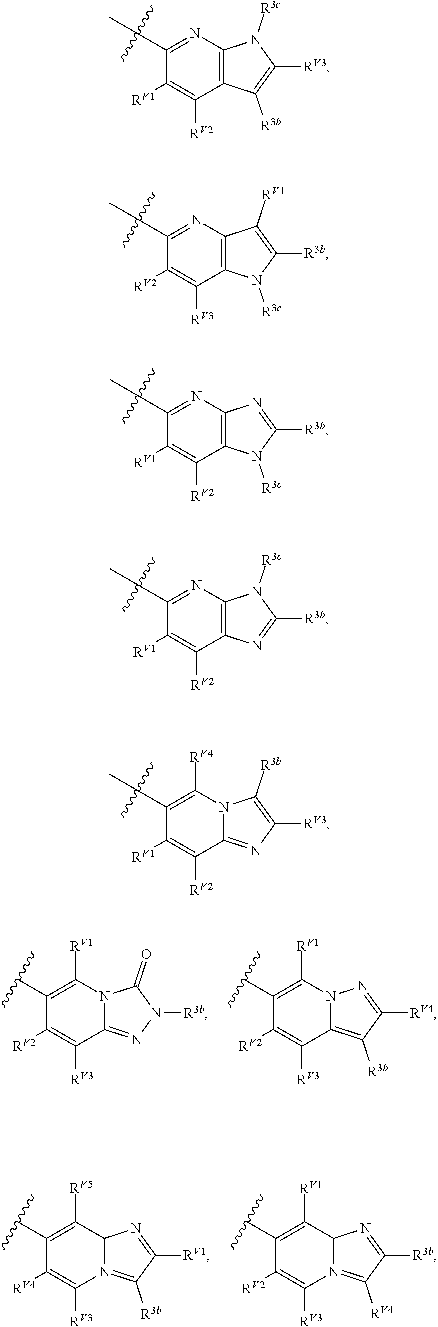 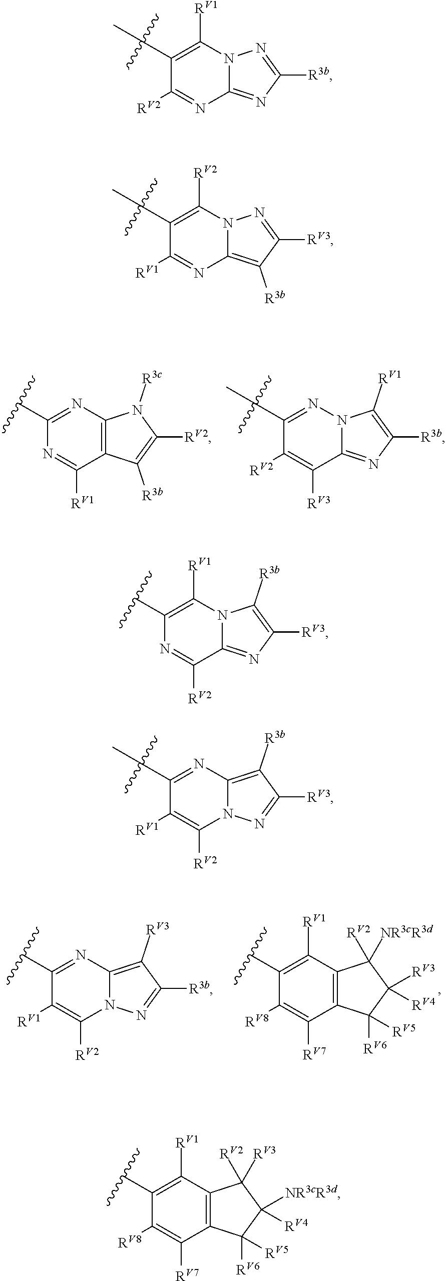 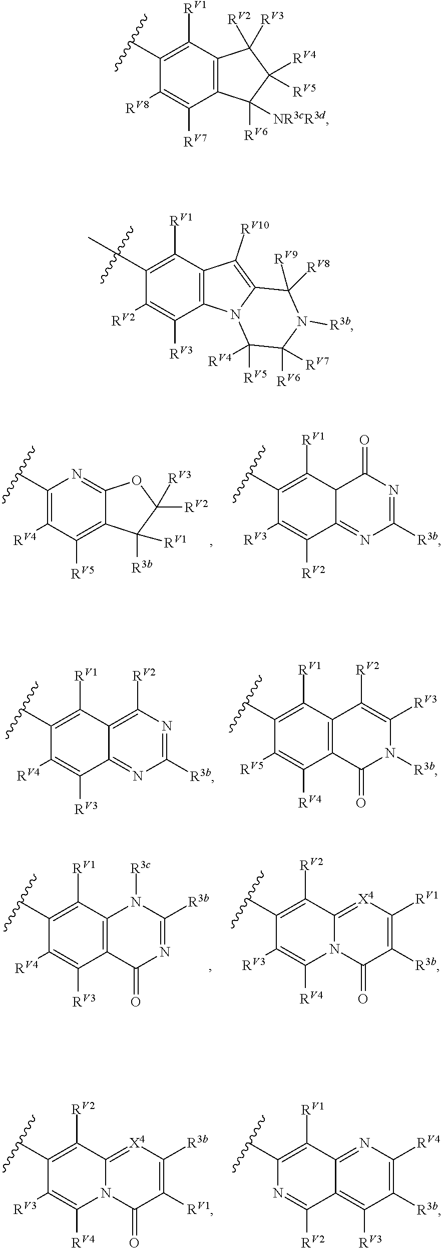 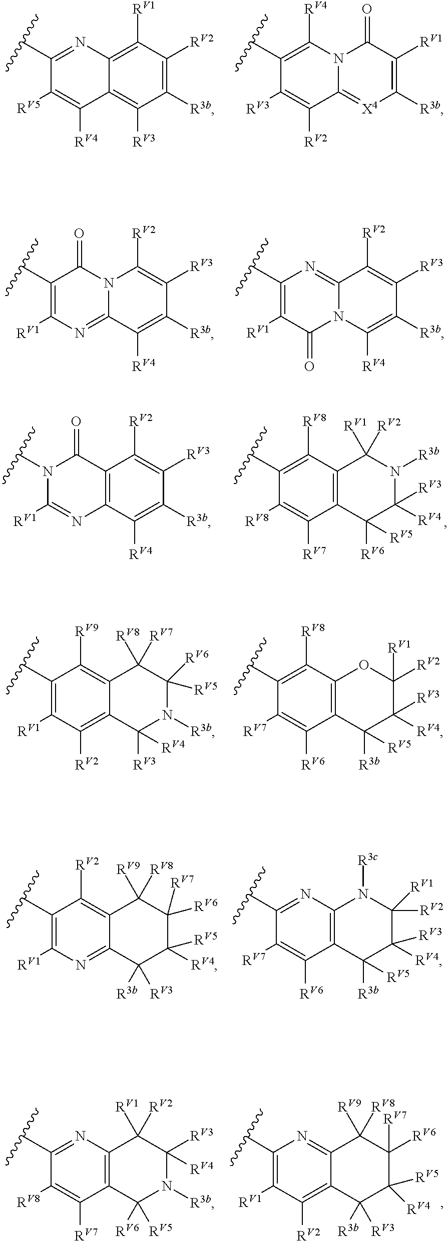 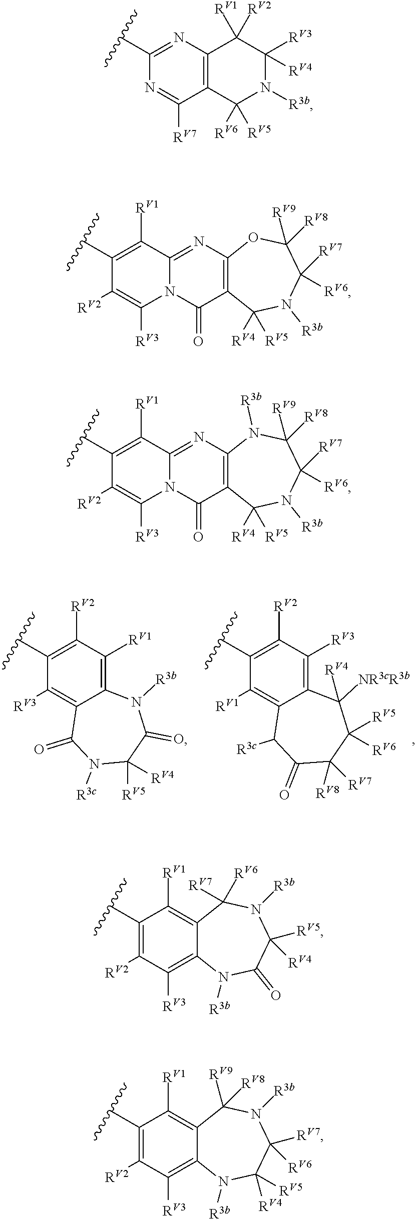 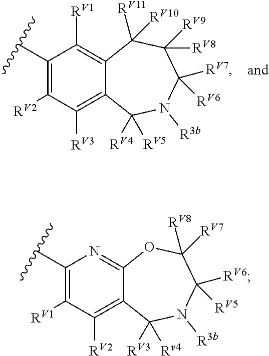
each occurrence of X⁴ is independently selected from the group consisting of CR^{X4} and N, wherein each occurrence of R^{X4} is independently selected from the group consisting of H, C₁-C₃ alkyl, C₃-C₅ cycloalkyl, C₁-C₃ alkoxy, cyano, halogen, C₁-C₃ haloalkyl, and C₁-C₃ haloalkoxy;
R^{2a} is selected from the group consisting of H, halogen, optionally substituted C₁-C₆ alkyl, optionally substituted C₃-C₅ cycloalkyl, —(CH₂)₁₋₃(optionally substituted phenyl), —(CH₂)₁₋₃(optionally substituted heteroaryl), —OR, —SR¹, —NR^{I}R^{I}, —O(CH₂)₁₋₃(optionally substituted phenyl), —O(CH₂)₁₋₃(optionally substituted heteroaryl), —(CH₂)₁₋₃C(═O)OR, —(CH₂)₁₋₃C(═O)NR′R′, —O(CH₂)₁₋₃C(═O)OR′, and —O(CH₂)₁₋₃C(═O)NR^{III}R^{III},
wherein each occurrence of R is independently H, C₁-C₆ alkyl, or C₃-C₈ cycloalkyl, wherein the alkyl or cycloalkyl is independently optionally substituted with at least one of halogen, —OH, C₁-C₆ alkoxy, —NH₂, —NH(C₁-C₆ alkyl), and —N(C₁-C₆ alkyl)(C₁-C₆ alkyl),
or two R can combine with the N atom to which they are bound to form 3-membered to 8-membered optionally substituted heterocyclyl;
R^{3a} is selected from the group consisting of —CHO, —C(O)OR^{III}, —C(═O)NR^{III}R^{III}, —C(═NR⁵)NR^{III}R^{III}, optionally substituted heterocyclyl, —(CH₂)₁₋₃(optionally substituted heterocyclyl), optionally substituted C₁-C₆ alkoxy, optionally substituted C₁-C₆ aminoalkyl, and optionally substituted C₁-C₆ hydroxyalkyl,
wherein each occurrence of R^{III} is independently H, C₁-C₆ alkyl, or C₃-C₈ cycloalkyl, wherein the alkyl or cycloalkyl is independently optionally substituted with at least one of halogen, —OH, C₁-C₆ alkoxy, —NH₂, —NH(C₁-C₆ alkyl), and —N(C₁-C₆ alkyl)(C₁-C₆ alkyl),
or two R^{III} can combine with the N atom to which they are bound to form 3-membered to 8-membered optionally substituted heterocyclyl,
or, if R^{3a} is —C(═NR⁵)NR^{III}R^{III}, then R⁵ and one R^{III} can combine to form 4-membered to 8-membered optionally substituted heterocyclyl,
or, if X³ is CR^{X3} and R^{3a} has a tetravalent a carbon, then optionally RX³ and the tetravalent a carbon of R^{3a} combine to form a 3-membered to to 8-membered carbocyclyl;
R^{3b} is selected from the group consisting of H, optionally substituted C₁-C₆ alkyl, optionally substituted C₁-C₆ alkoxy, optionally substituted C₁-C₆ aminoalkyl, optionally substituted C₁-C₆ hydroxyalkyl, optionally substituted C₃-C₈ cycloalkyl, —CHO, —C(O)OR^{1V}, —C(═O)NR^{IV}R^{IV}, —C(═NR⁵)NR^{1V}R^{1V}, —NR^{3′} (optionally substituted alkyl), optionally substituted heterocyclyl, and —(CH₂)₁₋₃(optionally substituted heterocyclyl),
wherein each occurrence of R^{IV} is independently H, C₁-C₆ alkyl, or C₃-C₈ cycloalkyl, wherein the alkyl or cycloalkyl is independently optionally substituted with at least one of halogen, —OH, C₁-C₆ alkoxy, —NH₂, —NH(C₁-C₆ alkyl), and —N(C₁-C₆ alkyl)(C₁-C₆ alkyl),
or two R^{1V} can combine with the N atom to which they are bound to form 3-membered to 8-membered optionally substituted heterocyclyl,
or, if R^{3b} is —C(═NR⁵)NR^{IV}R^{1V}, then R⁵ and one R^{1V} can combine to form 4-membered to 8-membered optionally substituted heterocyclyl;
each occurrence of R^{3c} is independently selected from the group consisting of H, optionally substituted C₁-C₆ alkyl, optionally substituted C₃-C₈ cycloalkyl, and optionally substituted C₁-C₆ acyl;
each occurrence of R^{3d} is independently selected from the group consisting of H, optionally substituted C₁-C₆ alkyl, optionally substituted C₃-C₈ cycloalkyl, and optionally substituted C₁-C₆ acyl;
R^{4a} is selected from the group consisting of H, halogen, cyano, C₁-C₃ alkyl, and C₃-C₈ cycloalkyl;
each occurrence of R⁵ is independently H, C₁-C₆ alkyl, or C₃-C₈ cycloalkyl, wherein the alkyl or cycloalkyl is independently optionally substituted with at least one of halogen, —OH, C₁-C₆ alkoxy, —NH₂, —NH(C₁-C₆ alkyl), and —N(C₁-C₆ alkyl)(C₁-C₆ alkyl),
each occurrence of R^{V1}, R^{V2}, R^{V3}, R^{V4}, R^{V5}, R^{V6}, R^{V7}, R^{V8}, R^{V9}, RV¹⁰, and R^{V11} is independently selected from the group consisting of H, C₁-C₃ alkyl, C₃-C₈ cycloalkyl, C₁-C₃ alkoxy, cyano, halogen, C₁-C₃ haloalkyl, and C₁-C₃ haloalkoxy; and any mixtures thereof.

2. The compound of claim 1, wherein R^{1a} is selected from the group consisting of hydrogen, methyl, cyano, trifluoromethyl, fluoro, and chloro, and R^{1b} is selected from the group consisting of hydrogen, methyl, cyano, trifluoromethyl, fluoro, and chloro, with the proviso that R^{1a} and R^{1b} are not simultaneously hydrogen.

3. The compound of claim 1, wherein R^{2a} is selected from the group consisting of halogen, C₁-C₆ alkoxy, —CH₂(optionally substituted pyridinyl), —O(CH₂)₁₋₃C(═O)OH, and —O(CH₂)₁₋₃C(═O)O(C₁-C₆ alkyl).

4. The compound of claim 1, wherein R^{3a} is identical to R^{3b}.

5. The compound of claim 1, wherein R^{3a} is selected from the group consisting of —CHO, —CH₂OH, —C(═NH)NH₂, —(CH₂)₀₋₁(optionally substituted piperidinyl), —(CH₂)₀₋₁(optionally substituted tetrahydropyrimidinyl), —(CH₂)₀₋₁(optionally substituted imidazolyl), —(CH₂)₀₋₁(optionally substituted dihydroimidazolyl), —C(═O)NR³(C₁-C₆ hydroxyalkyl), —(CH₂)₀₋₁NR³C(C₁-C₆ haloalkyl), —(CH₂)₀₋₁NR^{3C}(C₁-C₆ hydroxyalkyl), —(CH₂)₀₋₁N(C₁-C₆ hydroxyalkyl)(C₁-C₆ hydroxyalkyl), —O(C₁-C₆ aminoalkyl), —(CH₂)₀₋₁NR^{3C}(C₁-C₆ aminoalkyl), —(CH₂)₀₋₁NR^{3C}(C₁-C₆ acetamidoalkyl), —(CH₂)₀₋₁NR³—CH(COOH)(CH₂)₁₋₆OH, —(CH₂)₀₋₁NR^{3C}—CH(C(═O)OC₁-C₆ alkyl)(CH₂)₁₋₆OH, —(CH₂)₀₋₁NR³C—C(C₁-C₆ alkyl)(COOH)(CH₂)₁₋₆OH, —(CH₂)₀₋₁NR^{3C}—C(C₁-C₆ alkyl)(C(═O)OC₁-C₆ alkyl)(CH₂)₁₋₆OH, —(CH₂)₀₋₁NR³, (CH₂)₁₋₆-(optionally substituted pyridinyl), and —(CH₂)₀₋₁NR^{3C}(CH₂)₁₋₆-(optionally substituted 5-oxopyrrolidin-2-yl).

6. The compound of claim 1 wherein R^{3a} is selected from the group consisting of —C(═NH)NH₂, —(CH₂)₀₋₁(optionally substituted azetidinyl), —(CH₂)₀₋₁(optionally substituted pyrrolidinyl), —(CH₂)₀₋₁(optionally substituted piperidinyl), —(CH₂)₀₋₁(optionally substituted tetrahydropyrimidinyl), —(CH₂)₀₋₁(optionally substituted imidazolyl), and —(CH₂)₀₋₁(optionally substituted dihydroimidazolyl).

7. The compound of claim 6, wherein in R^{3a} the —C(═NH)NH₂, azetidinyl, pyrrolidinyl, piperidinyl, tetrahydropyrimidinyl, imidazolyl, or dihydroimidazolyl is optionally substituted with at least one selected from the group consisting of oxo (═O), C₁-C₆ alkyl, C₁-C₆ hydroxyalkyl, C₁-C₆ aminoalkyl, C₁-C₆ N-acylaminoalkyl, —(CH₂)₀₋₃C(═O)OH, —(CH₂)₀₋₃C(═O)O(C₁-C₆ alkyl), —OH, C₁-C₆ alkoxy, —O(CH₂)₀₋₃C(═O)OH, and —O(CH₂)₀₋₃C(═O)O(C₁-C₆ alkyl).

8. The compound of claim 1, wherein R^{3a} is selected from the group consisting of —CH₂CH₂OH, —CH₂NHCH₂CH₂OH, —CH₂CH₂NHCH₂CH₂OH, —CH₂NHCH(COOH)CH₂OH, —CH₂NHC(CH₃)(COOH)CH₂OH, —CH₂(2-carboxyazetidin-1-yl), —CH₂(3-carboxyazetidin-1-yl), —CH₂(2-carboxypyrrolidin-1-yl), —CH₂(3-carboxypyrrolidin-1-yl), —CH₂(2-carboxypiperidin-1-yl), —CH₂(3-carboxypiperidin-1-yl), —CH₂(4-carboxypiperidin-1-yl), and —CH₂NHCH₂-(5-oxopyrrolin-2-yl).

9. The compound of claim 1, wherein R^{3b} is selected from the group consisting of —CHO, —CH₂OH, —C(═NH)NH₂, —(CH₂)₀₋₁(optionally substituted piperidinyl), —(CH₂)₀₋₁(optionally substituted tetrahydropyrimidinyl), —(CH₂)₀₋₁(optionally substituted imidazolyl), —(CH₂)₀₋₁(optionally substituted dihydroimidazolyl), —C(═O)NH(C₁-C₆ hydroxyalkyl), —CH₂NH(C₁-C₆ haloalkyl), —CH₂NH(C₁-C₆ hydroxyalkyl), —CH₂N(C₁-C₆ hydroxyalkyl)(C₁-C₆ hydroxyalkyl), —CH₂NH(C₁-C₆ aminoalkyl), —CH₂NH(C₁-C₆ acetamidoalkyl), —CH₂NH—CH(COOH)(CH₂)₁₋₆OH, —CH₂NH—CH(C(═O)OC₁-C₆alkyl)(CH₂)₁₋₆OH, —CH₂NH—C(C₁-C₆ alkyl)(COOH)(CH₂)₁₋₆₀H, —CH₂NH—C(C₁-C₆ alkyl)(C(═O)OC₁-C₆alkyl)(CH₂)₁₋₆₀H, and —CH₂N(C₀-C₆ alkyl)-(CH₂)₁₋₆-(optionally substituted 5-oxopyrrolidin-2-yl).

10. The compound of claim 1, wherein R^{3b} is selected from the group consisting of —C(═NH)NH₂, —(CH₂)₀₋₄(optionally substituted azetidinyl), —(CH₂)₀₋₄(optionally substituted pyrrolidinyl), —(CH₂)₀₋₄(optionally substituted piperidinyl), —(CH₂)₀₋₁(optionally substituted tetrahydropyrimidinyl), —(CH₂)₀₋₄(optionally substituted imidazolyl), and —(CH₂)₀₋₄(optionally substituted dihydroimidazolyl).

11. The compound of claim 10, wherein in R^{3b} the —C(═NH)NH₂, azetidinyl, pyrrolidinyl, piperidinyl, tetrahydropyrimidinyl, imidazolyl, or dihydroimidazolyl is optionally substituted with at least one selected from the group consisting of oxo (═O), C₁-C₆ alkyl, C₁-C₆ hydroxyalkyl, C₁-C₆ aminoalkyl, C₁-C₆ N-acylaminoalkyl, —(CH₂)₀₋₃C(═O)OH, —(CH₂)₀₋₃C(═O)O(C₁-C₆ alkyl), —OH, C₁-C₆ alkoxy, —O(CH₂)₀₋₃C(═O)OH, or —O(CH₂)₀₋₃C(═O)O(C₁-C₆ alkyl).

12. The compound of claim 1, wherein R^{3b} is selected from the group consisting of —CH₂CH₂OH, —CH₂NHCH₂CH₂OH, —CH₂CH₂NHCH₂CH₂OH, —CH₂NHCH(COOH)CH₂OH, —CH₂NHC(CH₃)(COOH)CH₂OH, —CH₂(2-carboxyazetidin-1-yl), —CH₂(3-carboxyazetidin-1-yl), —CH₂(2-carboxypyrrolidin-1-yl), —CH₂(3-carboxypyrrolidin-1-yl), —CH₂(2-carboxypiperidin-1-yl), —CH₂(3-carboxypiperidin-1-yl), —CH₂(4-carboxypiperidin-1-yl), and —CH₂NHCH₂-(5-oxopyrrolin-2-yl).

13. The compound of claim 1, wherein R^{4a} is chloro.

14. The compound of claim 1, which is selected from the group consisting of:

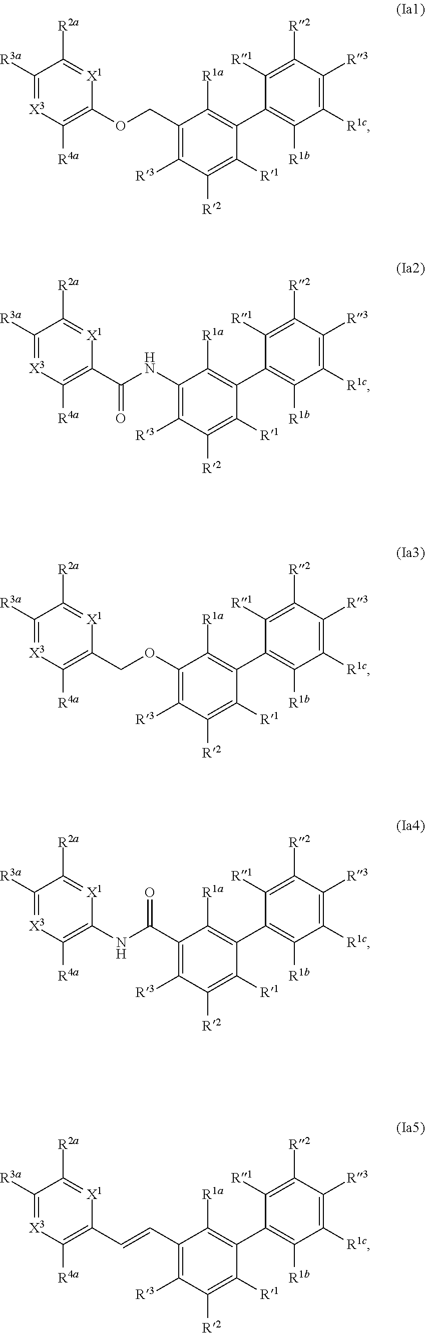

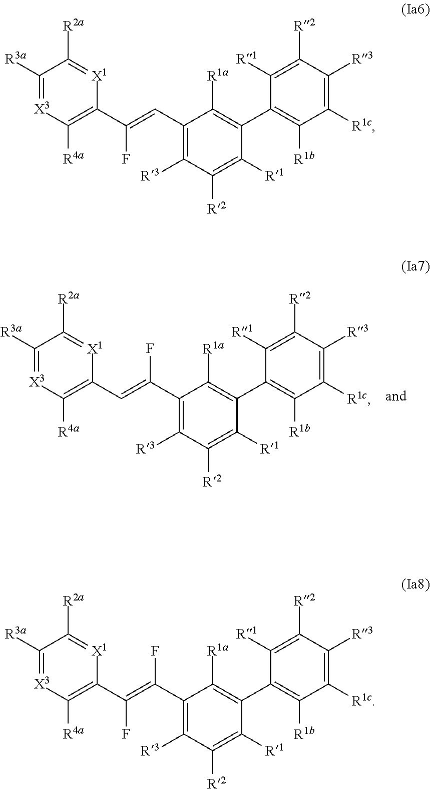

15. The compound of claim 1, wherein R^{1c} is selected from the group consisting of: 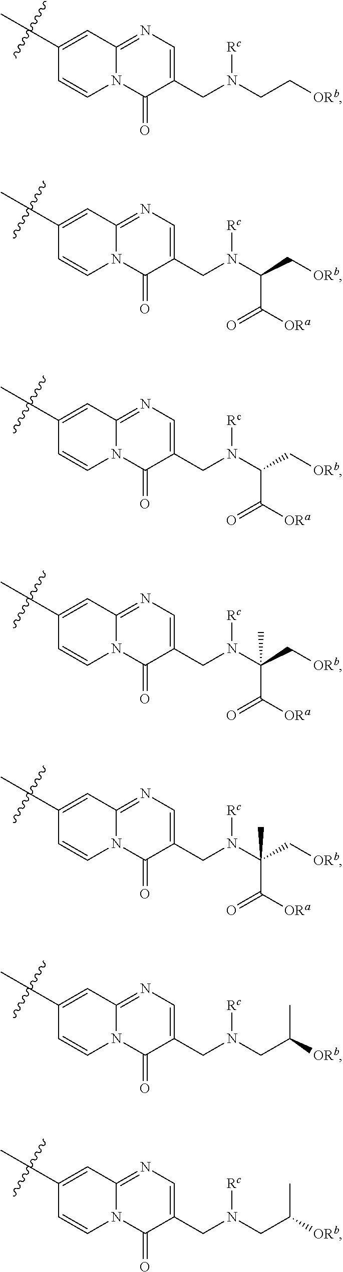 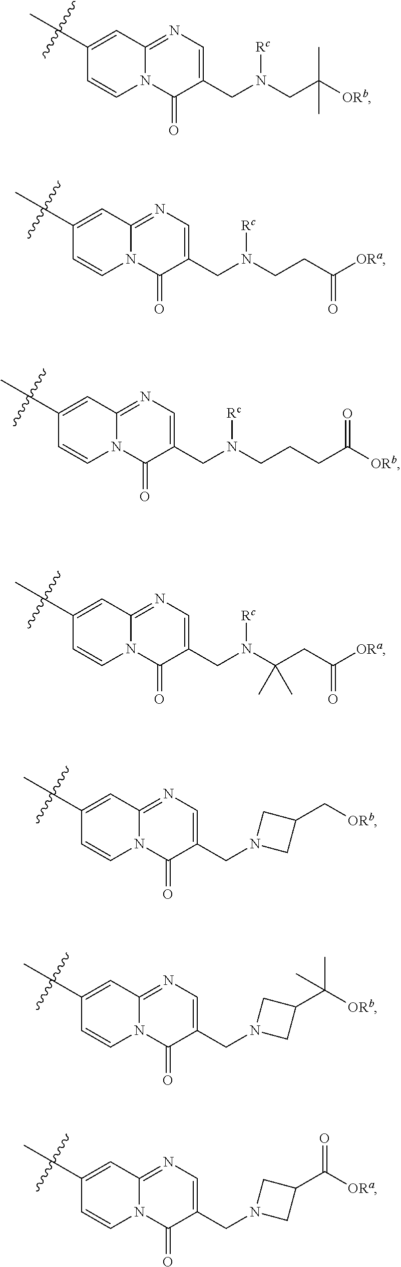 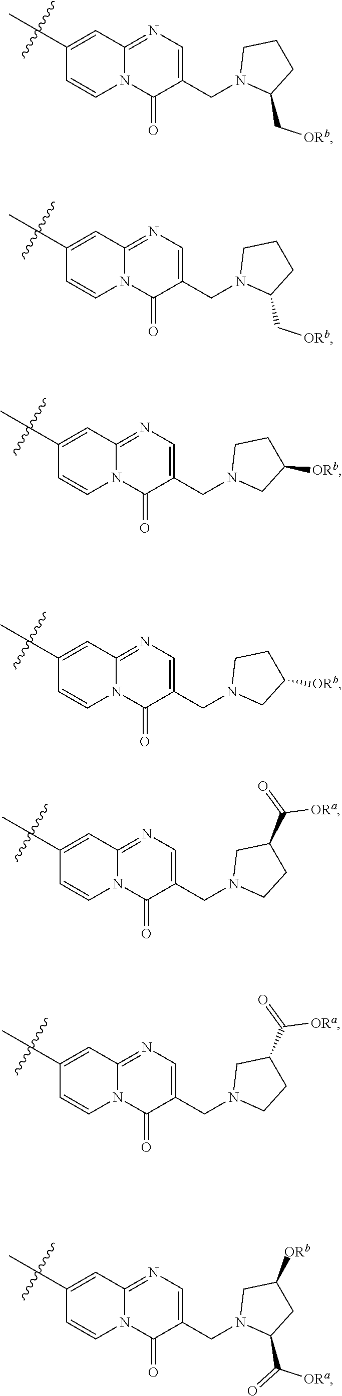 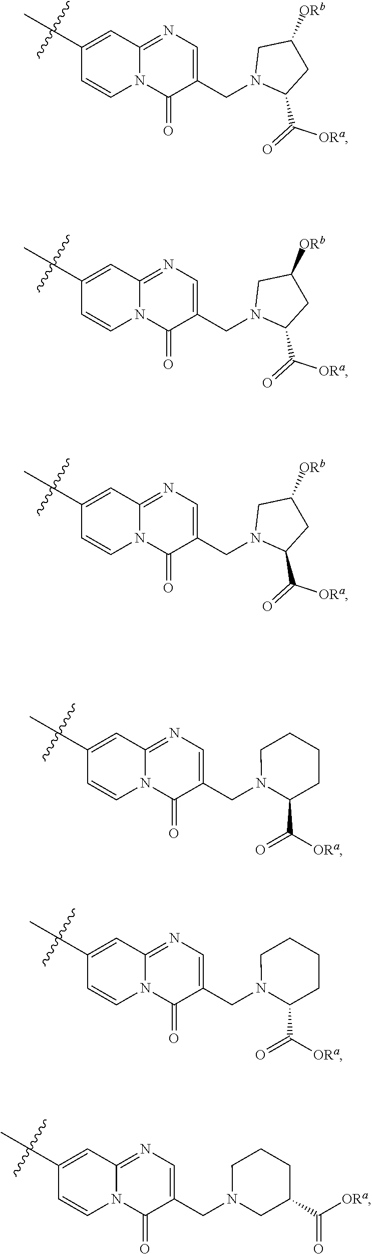 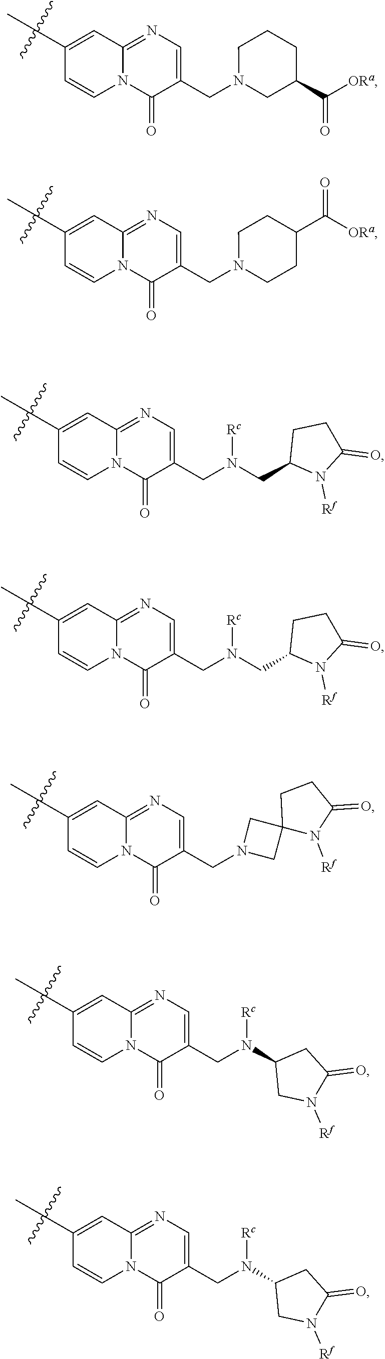 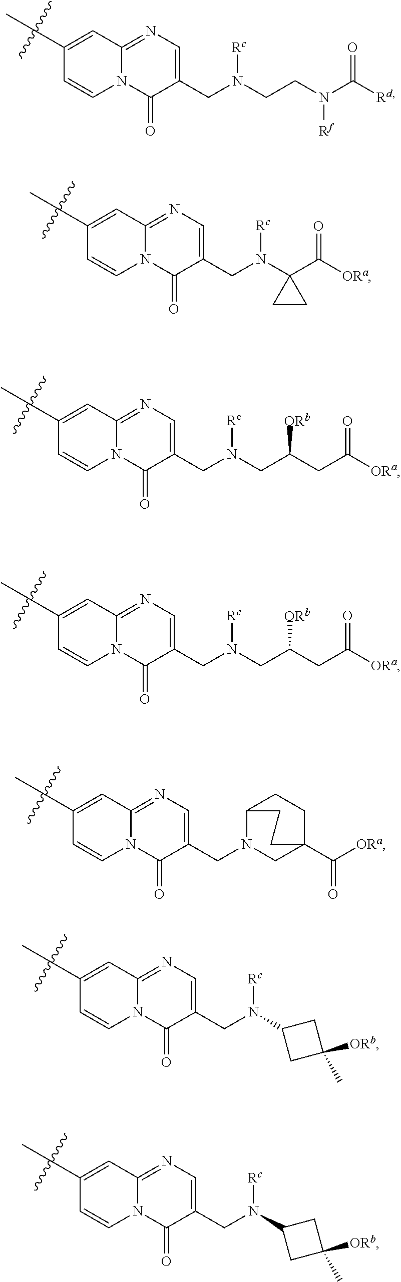 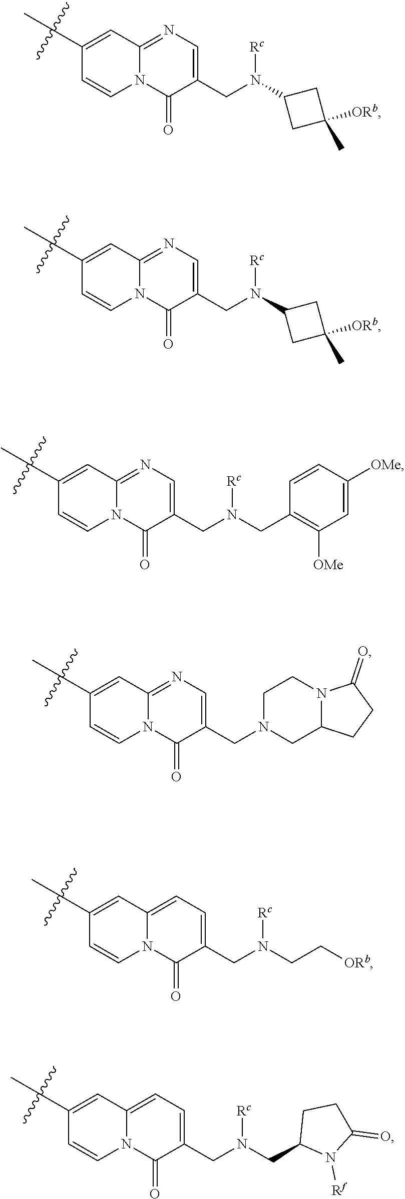 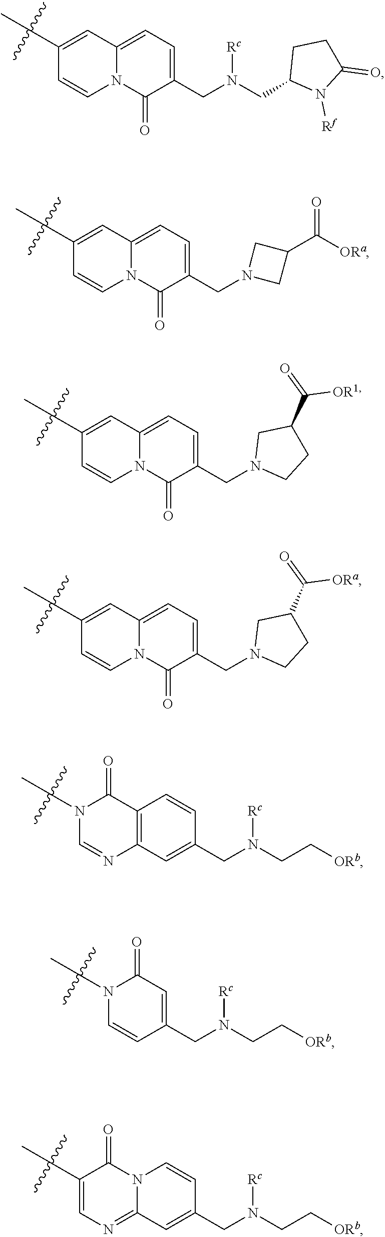 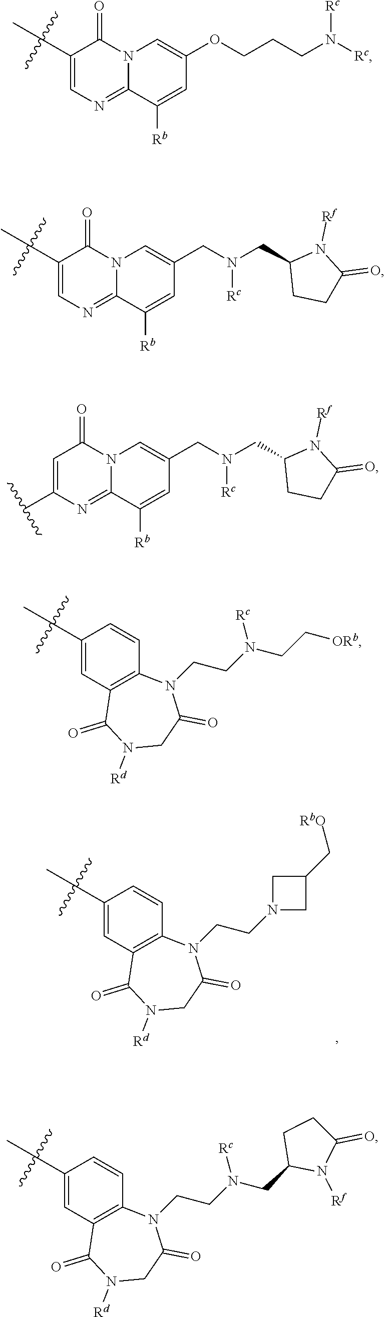 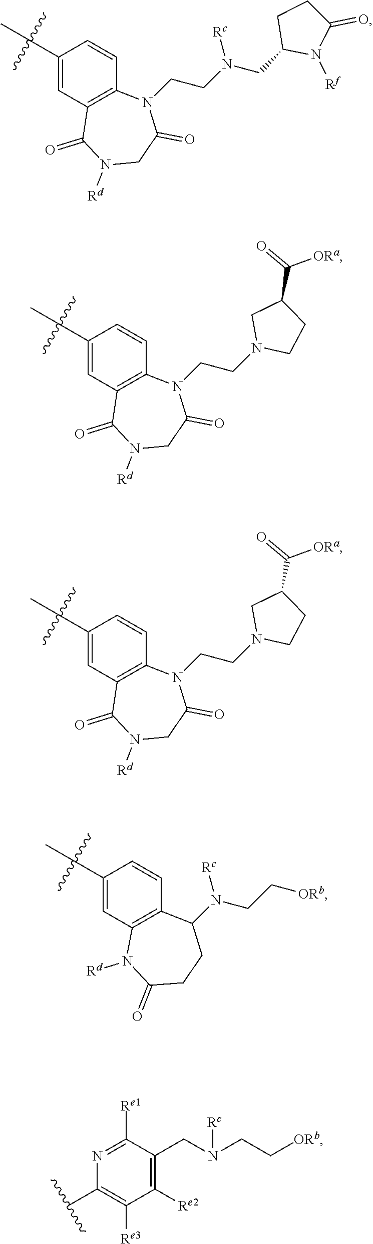 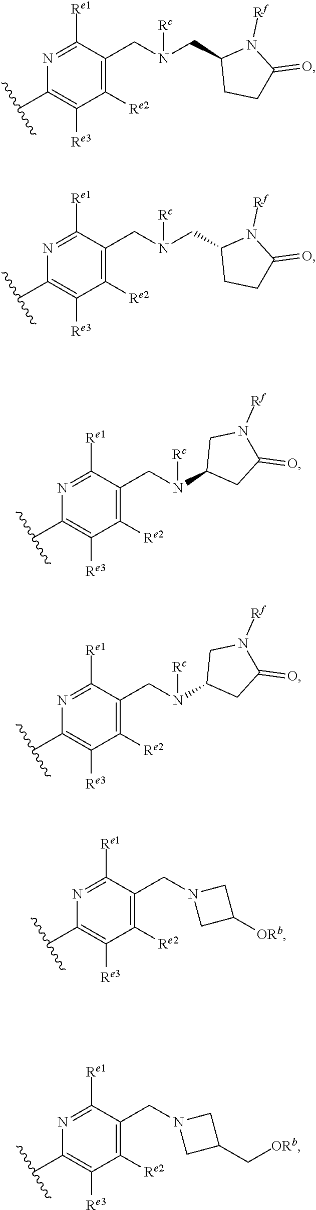 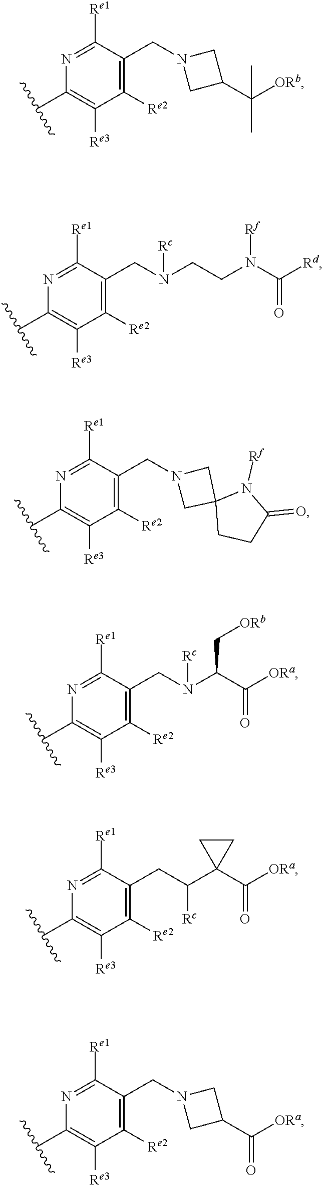 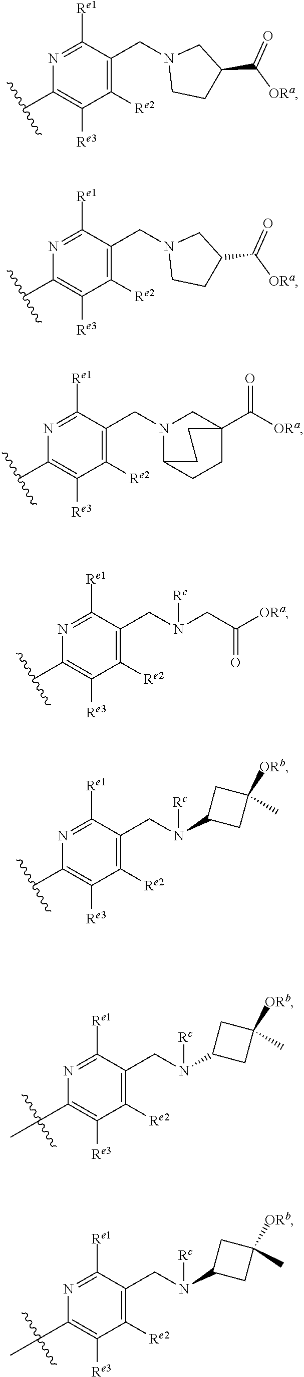 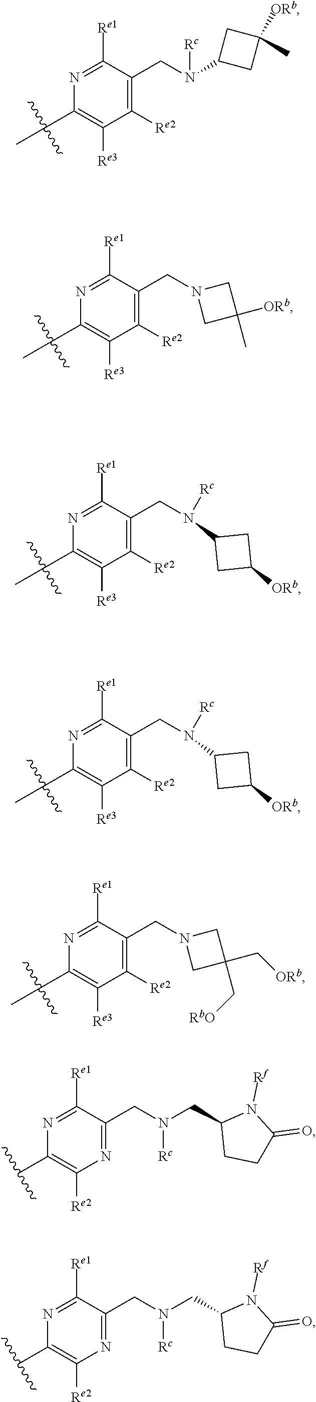 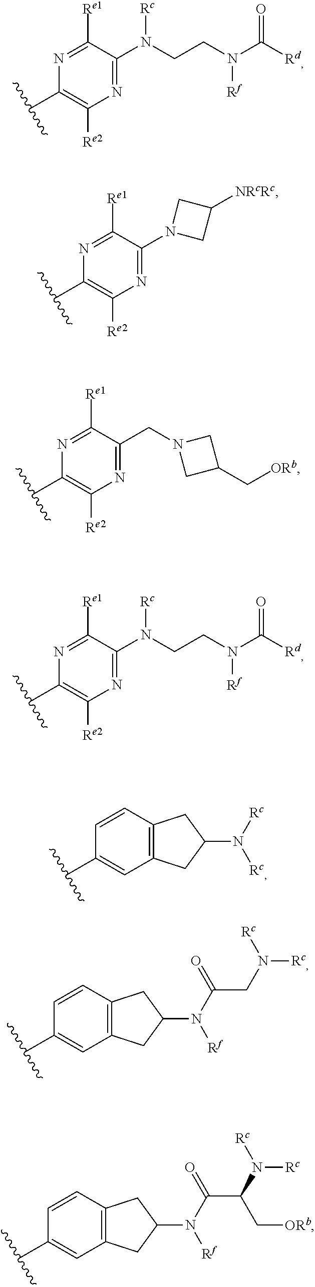 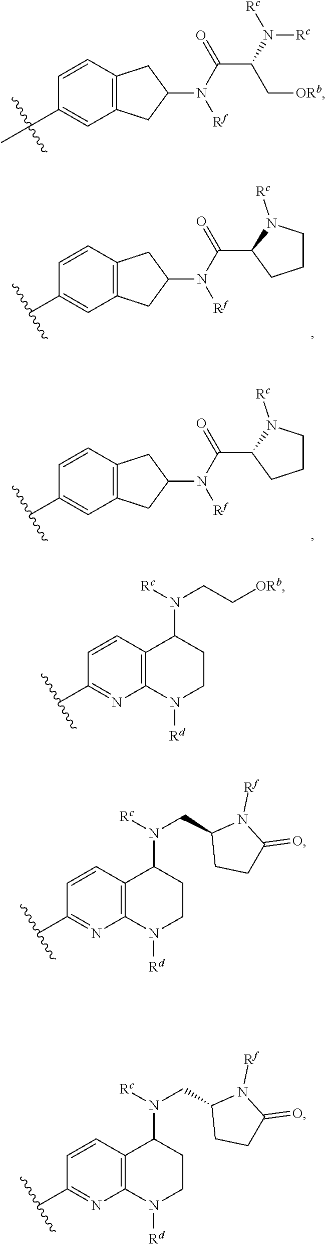 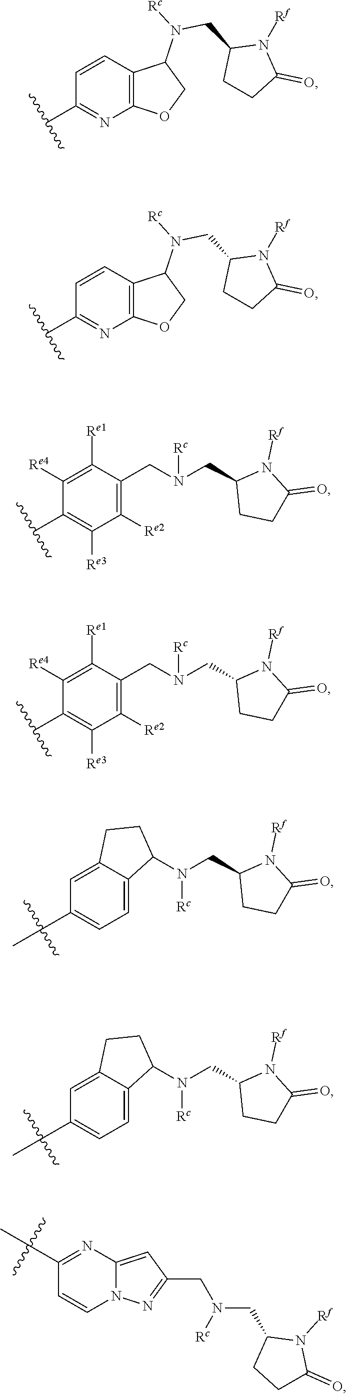 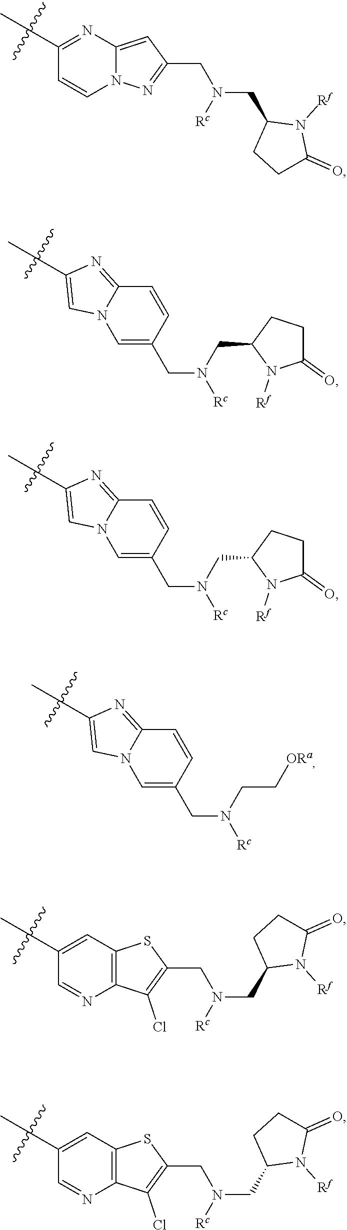 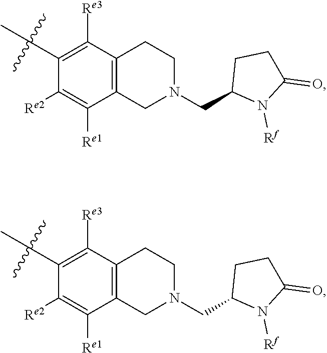
wherein:
each occurrence of R^{a}, R^{b}, R^{c}, R^{d}, and R is independently selected from the group consisting of H, optionally substituted C₁-C₆ alkyl, and optionally substituted C₃-C₈ cycloalkyl; and
each occurrence of R^{e1}, R^{e2}, R^{e3}, and R^{e4} is independently selected from the group consisting of H, F, Cl, Br, C₁-C₆ alkyl, C₁-C₆ haloalkyl, C₁-C₆ alkoxy, C₁-C₆ haloalkoxy, C₁-C₆ thioalkoxy, C₃-C₈ cycloalkyl, C₃-C₈ halocycloalkyl, C₃-C₈ cycloalkoxy, C₃-C₈ halocycloalkoxy, C₃-C₈ thioalkoxy, and NR^{g}R^{g},
wherein each occurrence of R^{g} is independently selected from the group consisting of H, optionally substituted C₁-C₆ alkyl, and optionally substituted C₃-C₈ cycloalkyl.

16. The compound of claim 1, wherein R^{1d} is selected from the group consisting of: 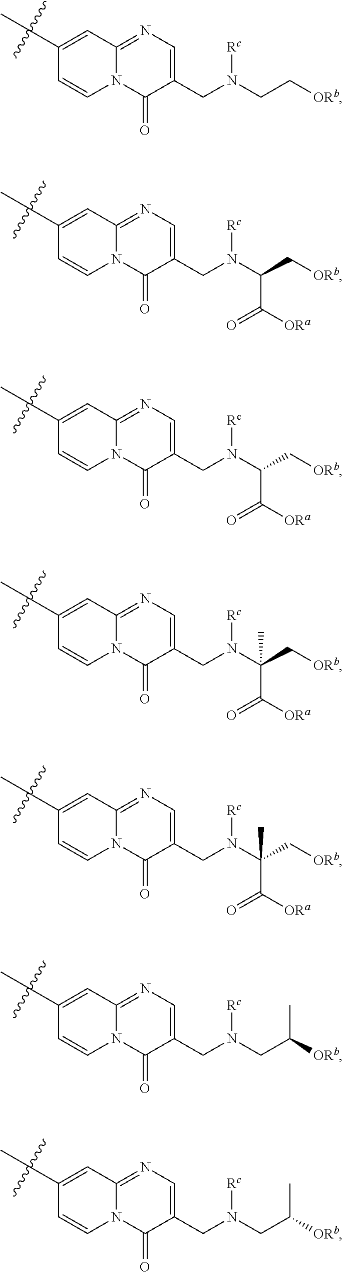 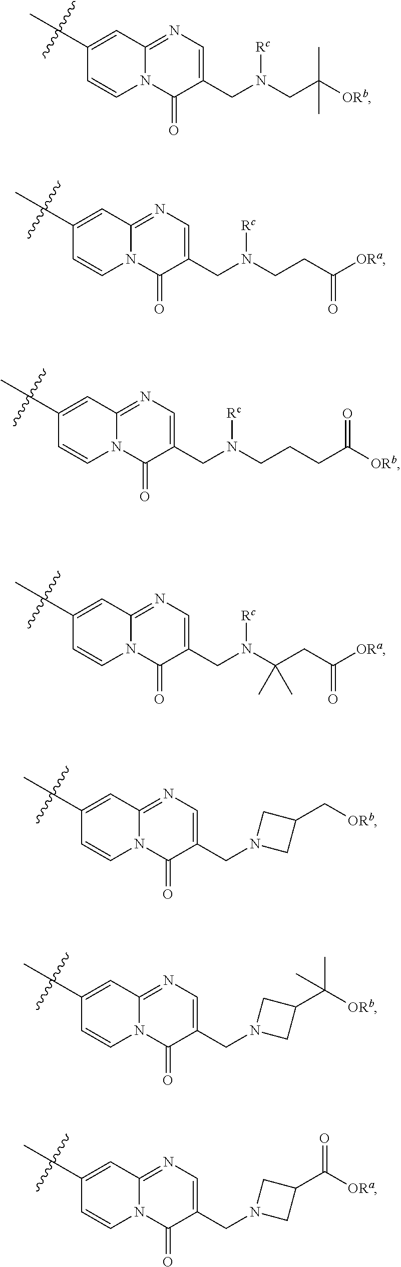 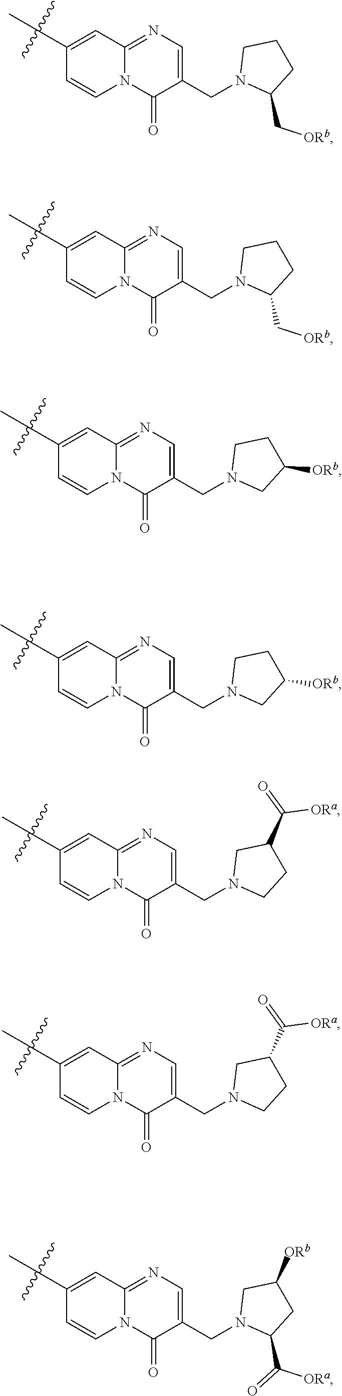 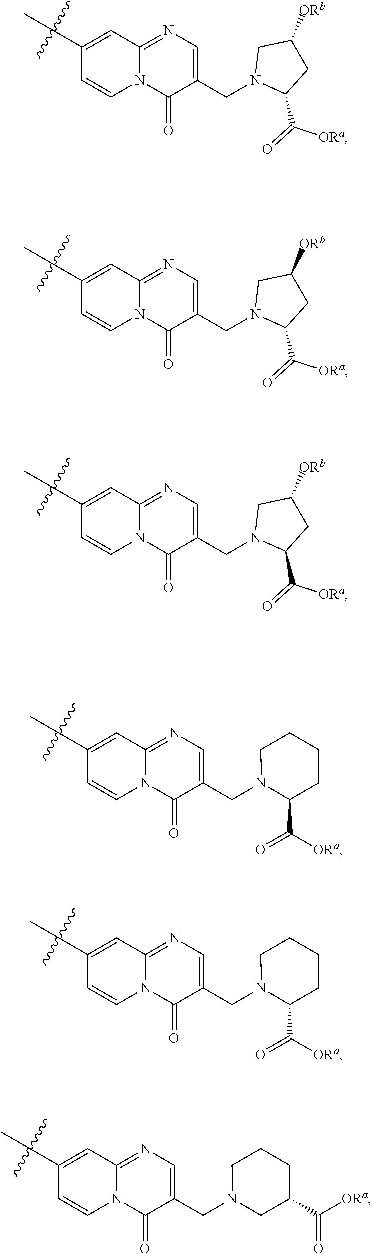 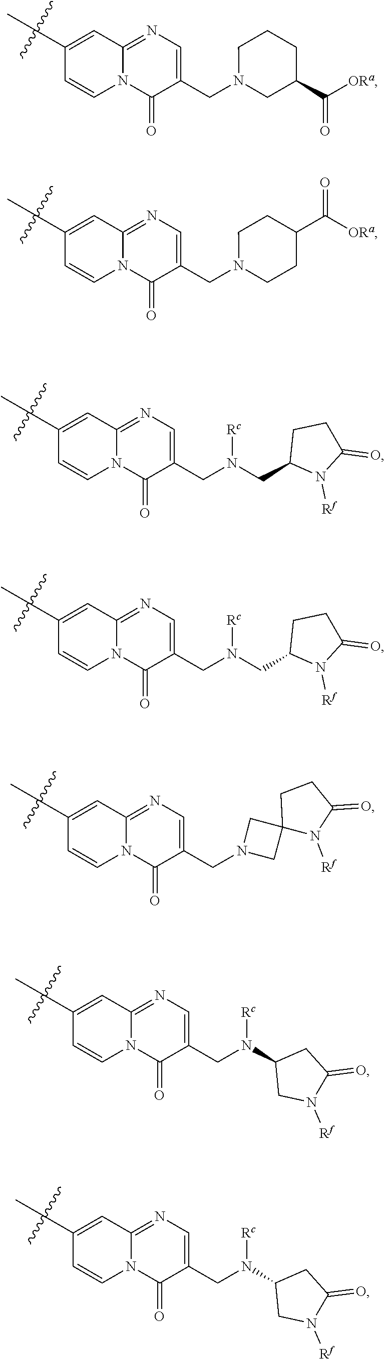 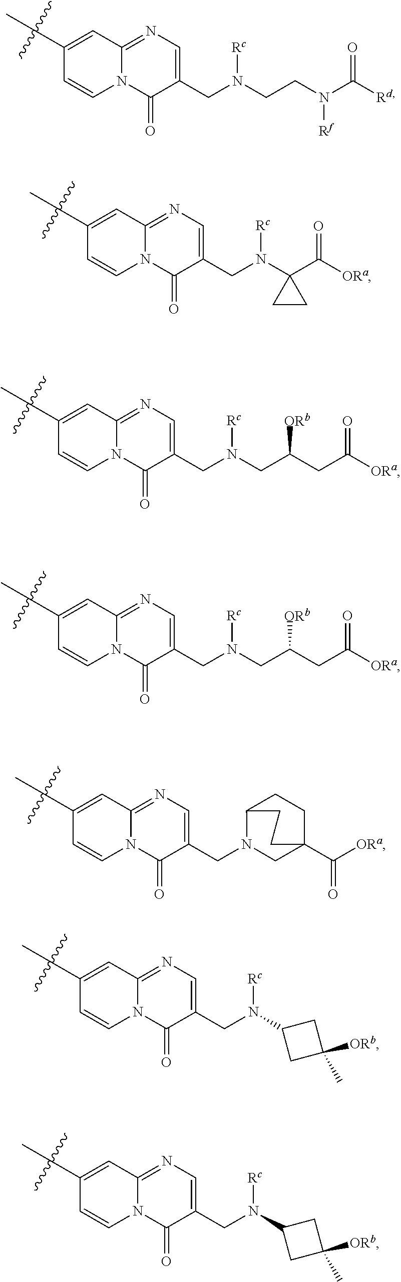 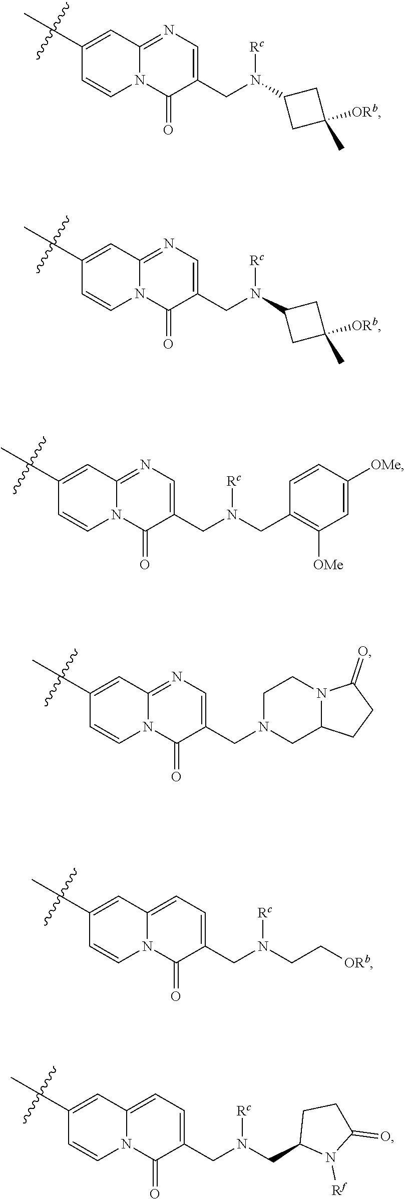 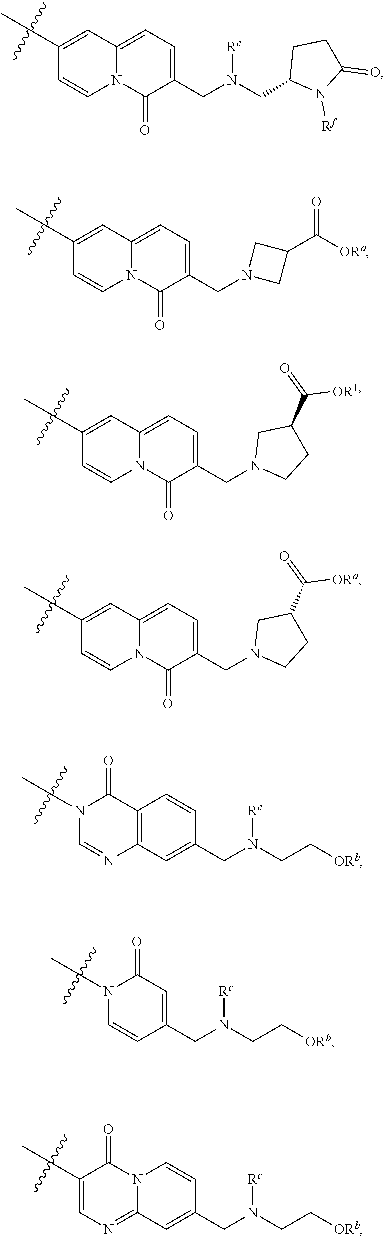 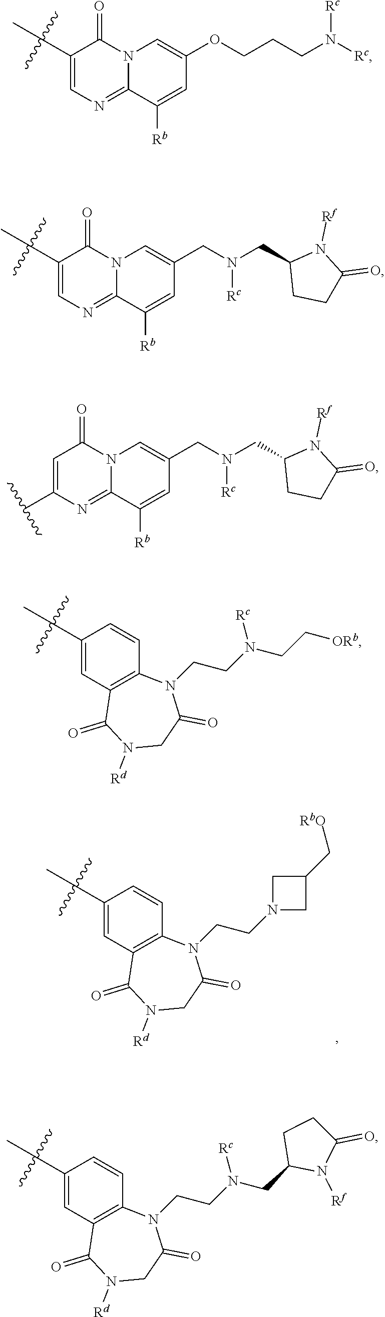 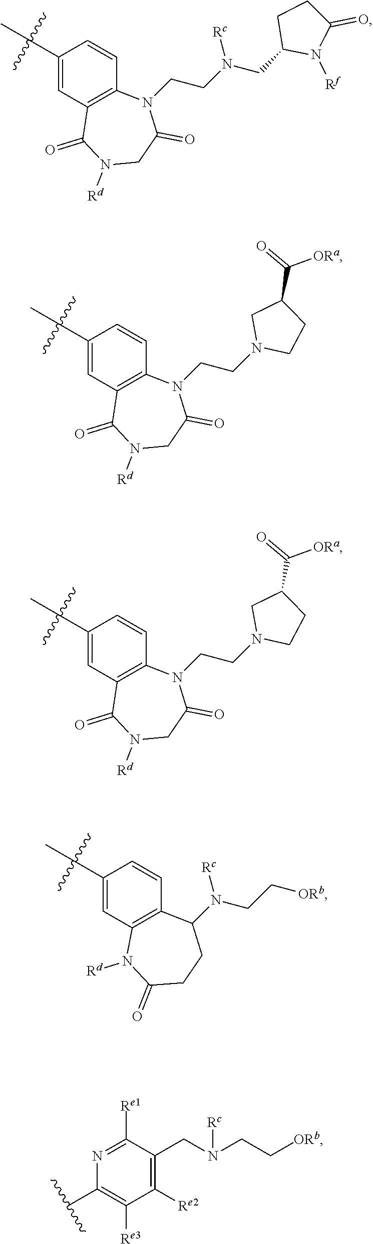 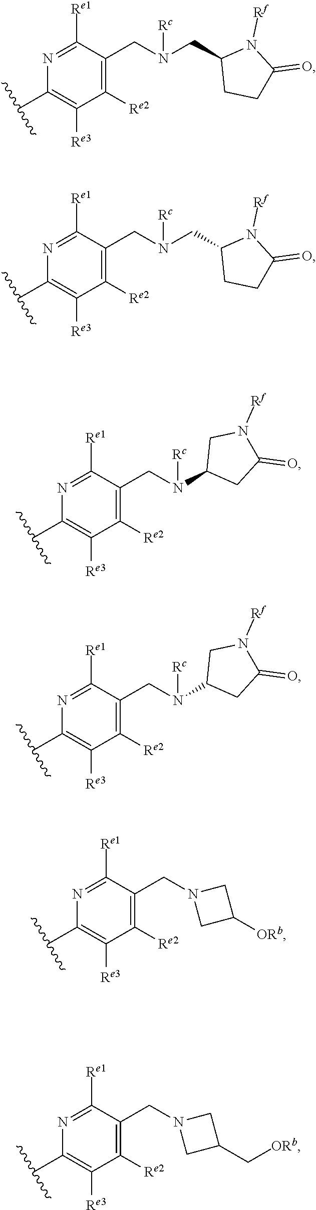 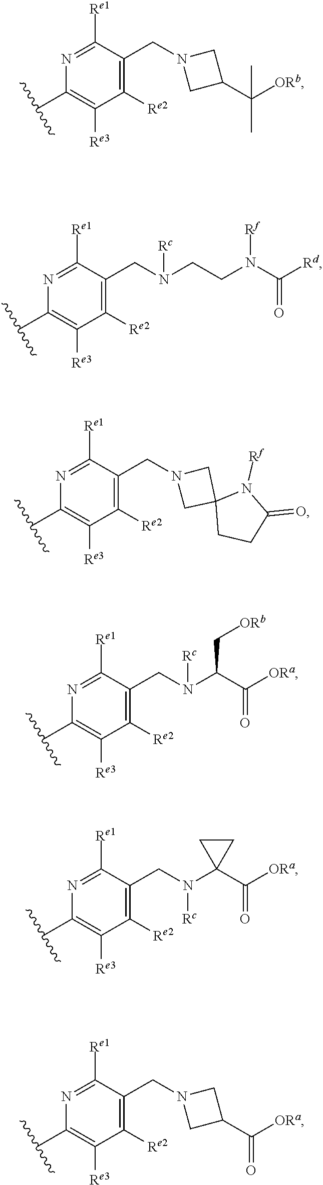 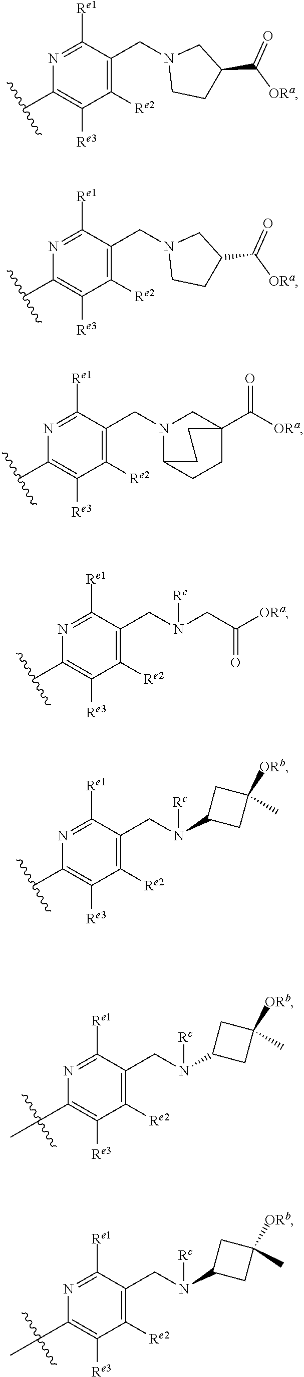 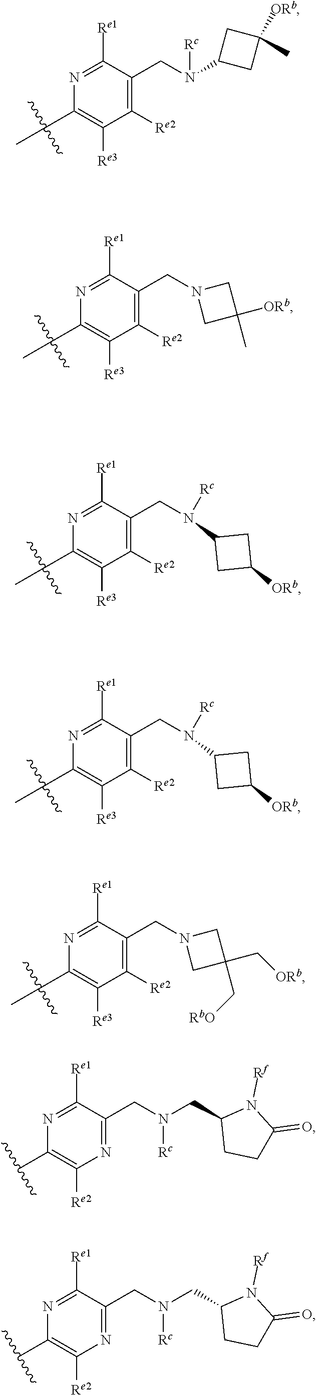 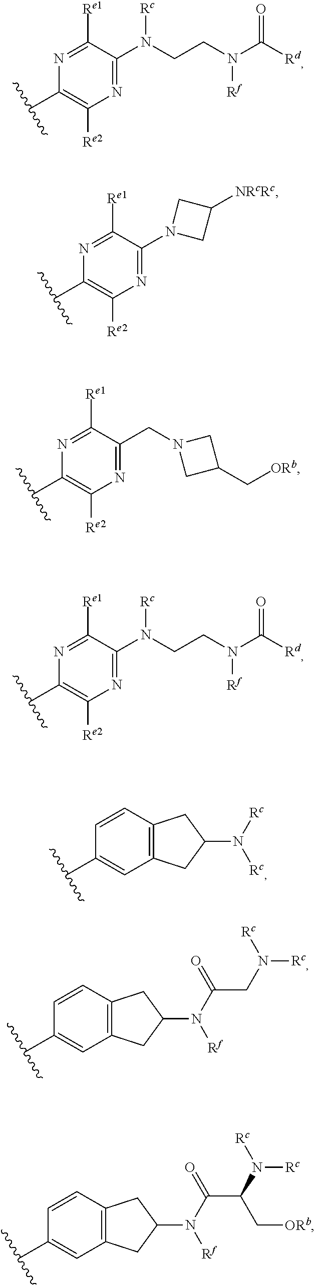 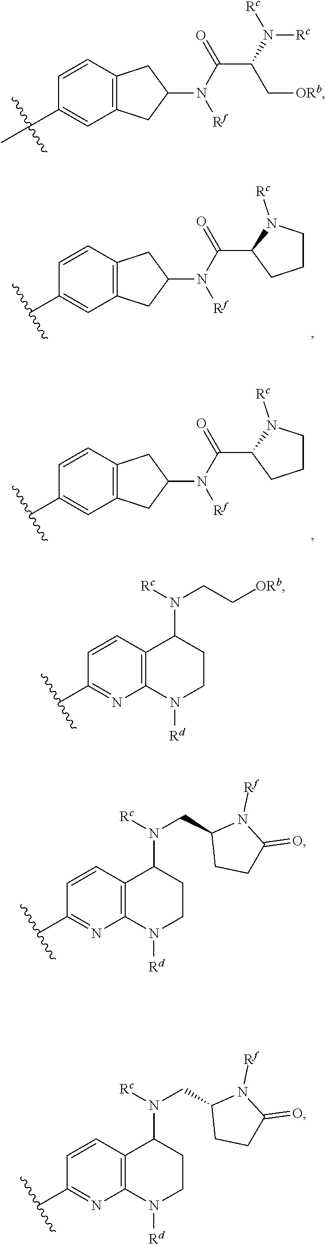 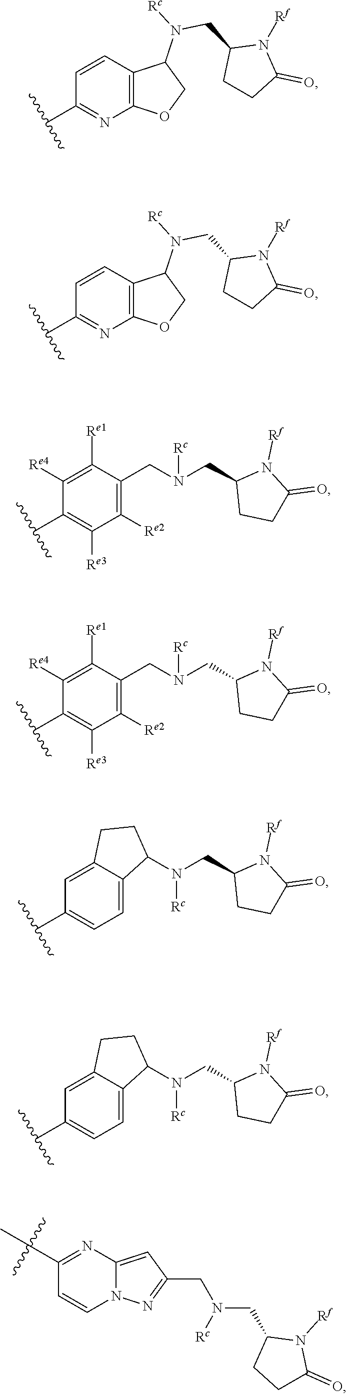 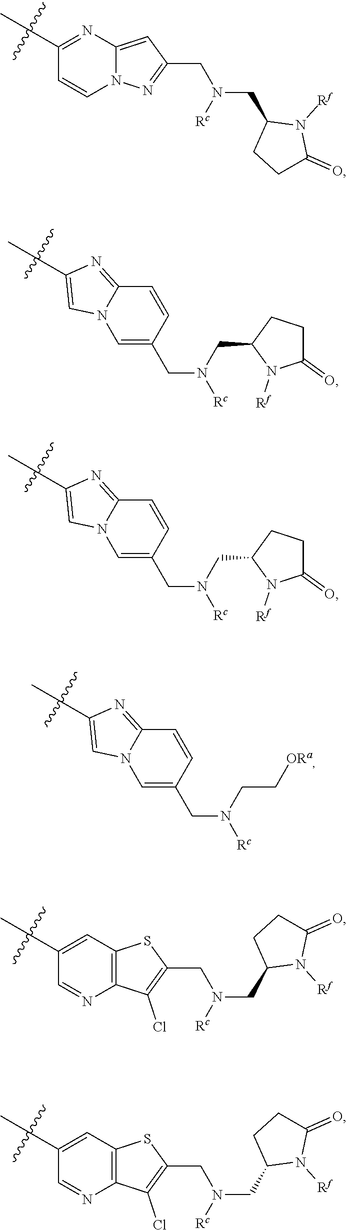 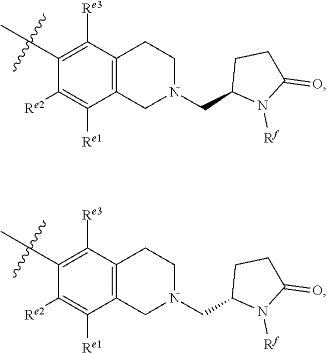
wherein:
each occurrence of R^{a}, R^{b}, R^{c}, R^{d}, and R is independently selected from the group consisting of H, optionally substituted C₁-C₆ alkyl, and optionally substituted C₃-C₈ cycloalkyl; and
each occurrence of R^{e1}, R^{e2}, R^{e3}, and R^{e4} is independently selected from the group consisting of H, F, Cl, Br, C₁-C₆ alkyl, C₁-C₆ haloalkyl, C₁-C₆ alkoxy, C₁-C₆ haloalkoxy, C₁-C₆ thioalkoxy, C₃-C₅ cycloalkyl, C₃-C₅ halocycloalkyl, C₃-C₈ cycloalkoxy, C₃-C₈ halocycloalkoxy, C₃-C₈ thioalkoxy, and NR^{g}R^{g},
wherein each occurrence of R^{g} is independently selected from the group consisting of H, optionally substituted C₁-C₆ alkyl, or optionally substituted C₃-C₈ cycloalkyl.

17. The compound of claim 1, wherein at least one of R^{3a} and R^{3b} is selected from the group consisting of:
—NR^{b}R^{c}, —CH₂NR^{b}R^{c}, —NR^{a}C(O)CH₂NR^{b}R^{c}, —NR^{a}C(O)CH(NR^{b}R^{c})CH₂OR^{d}, 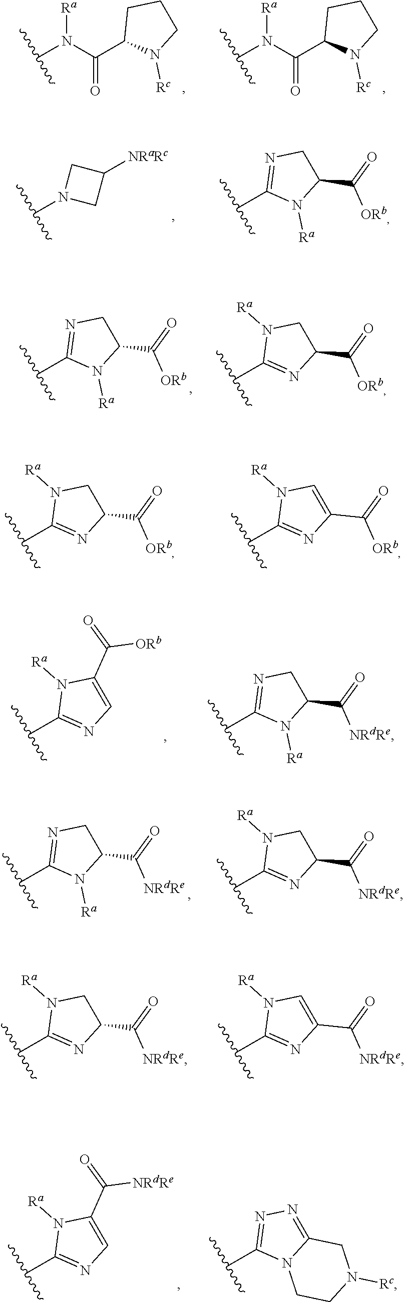 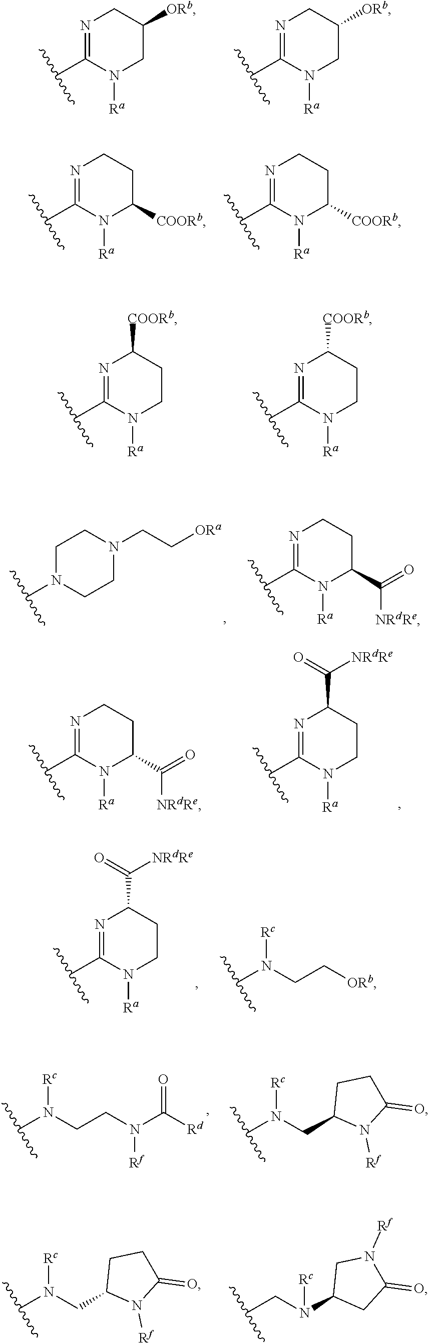 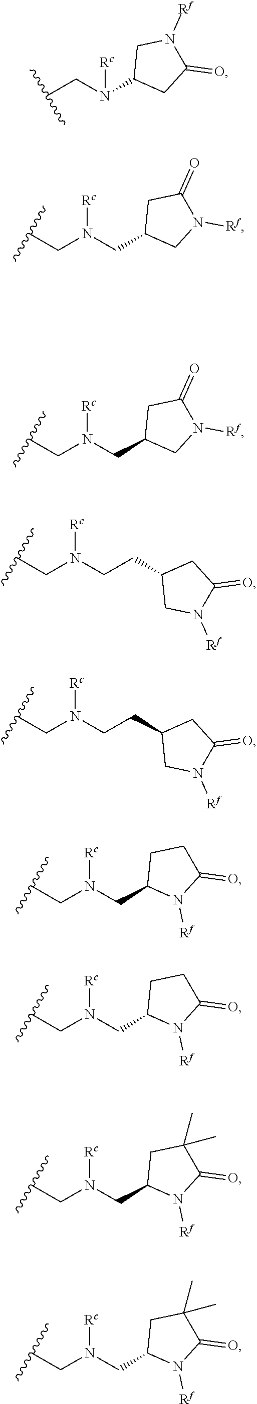 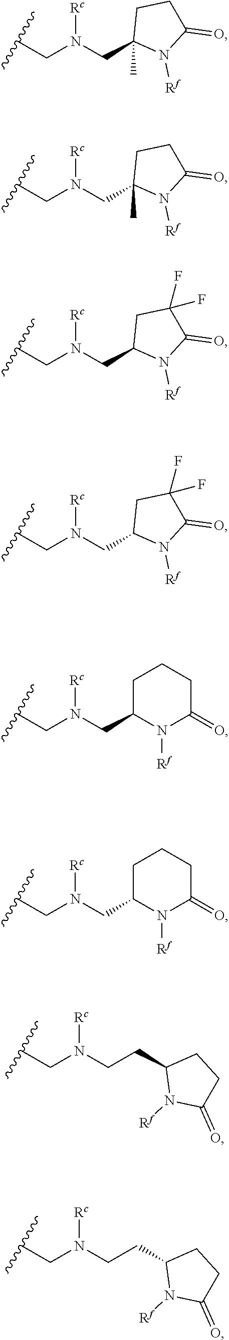 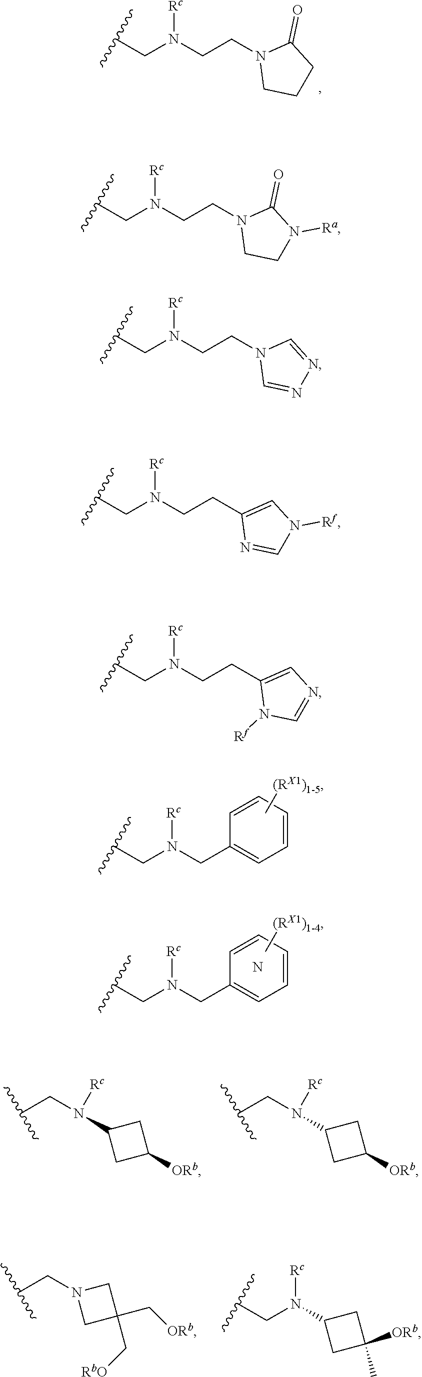 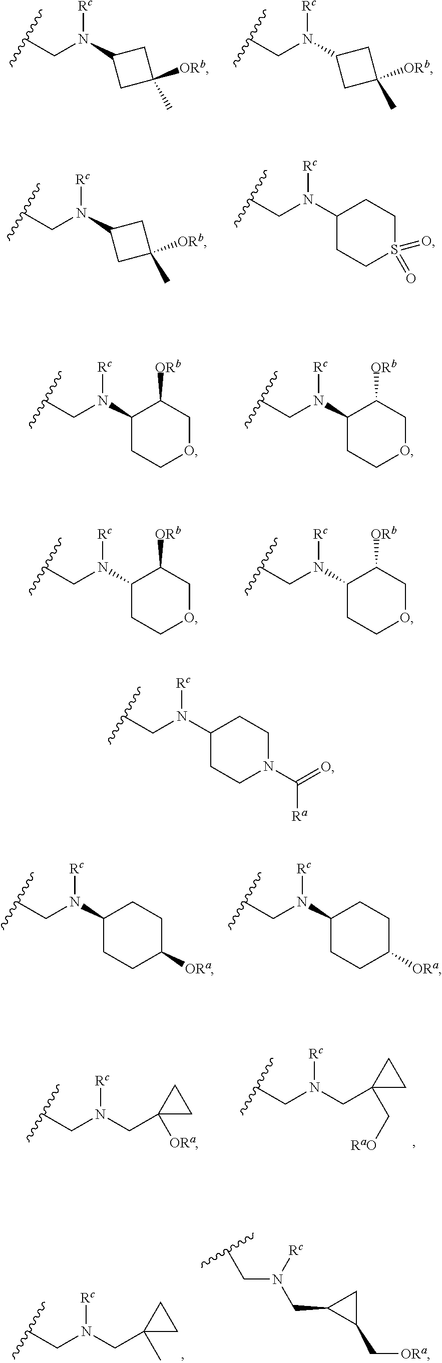 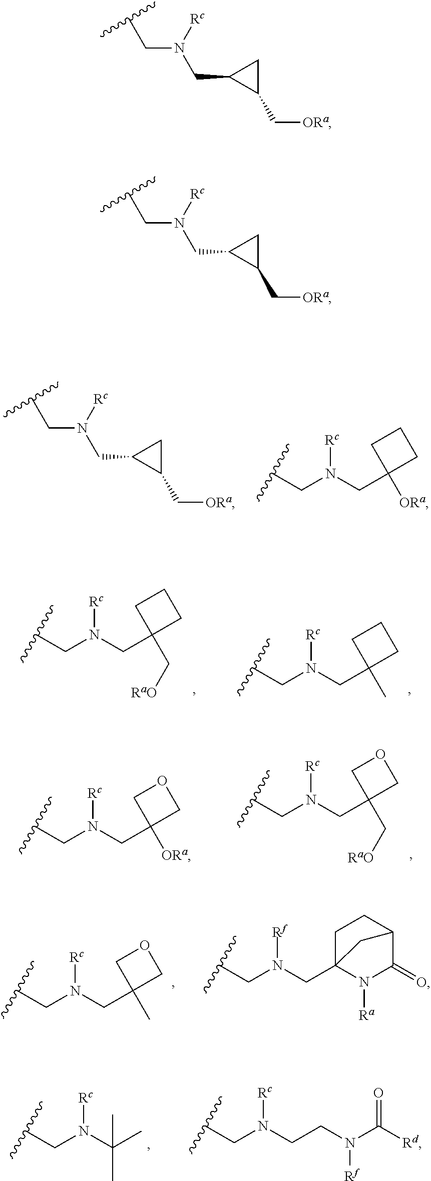 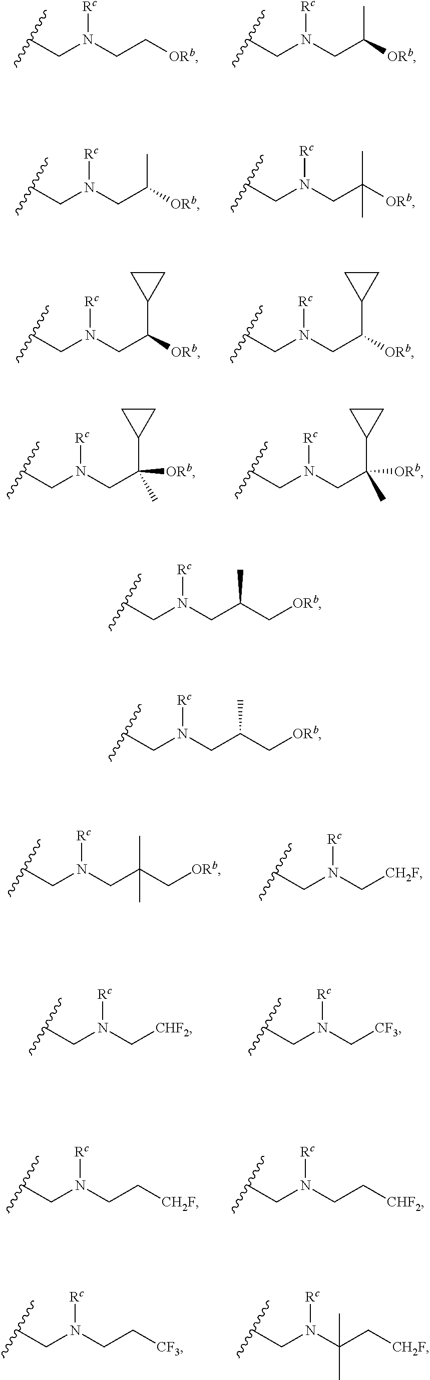 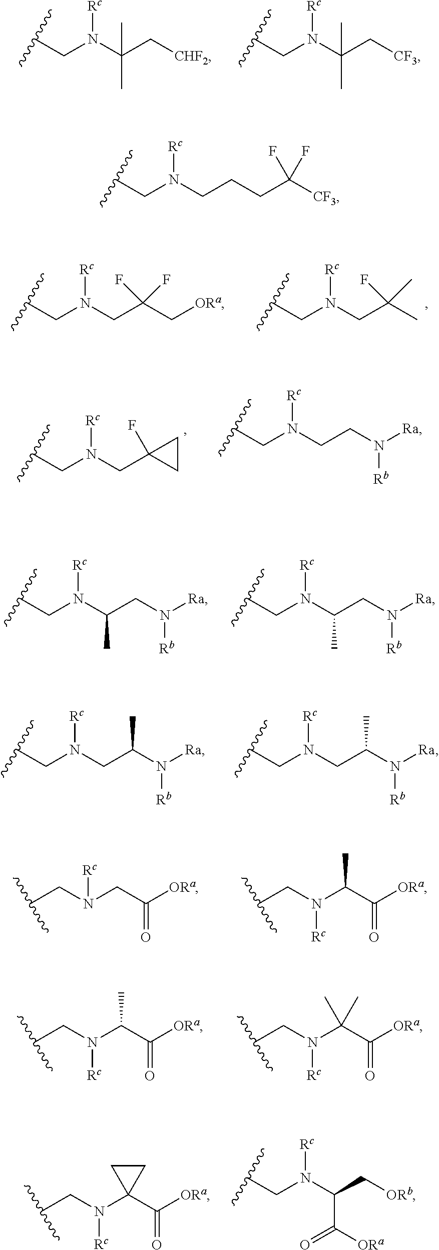 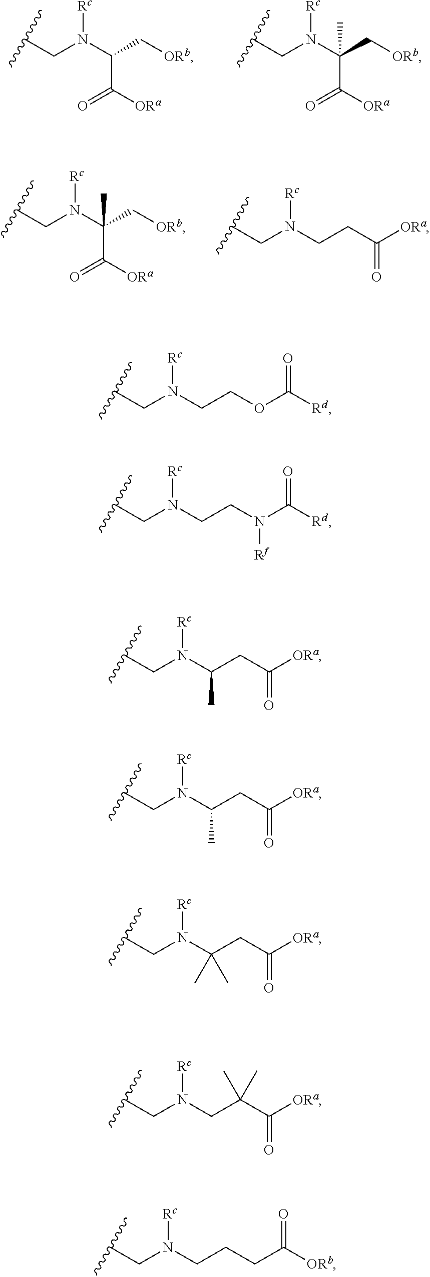 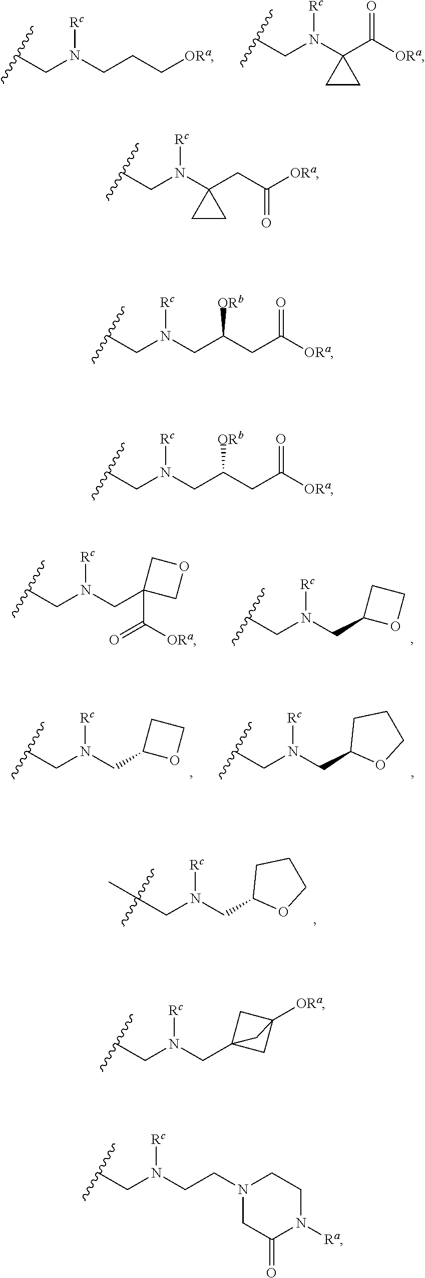 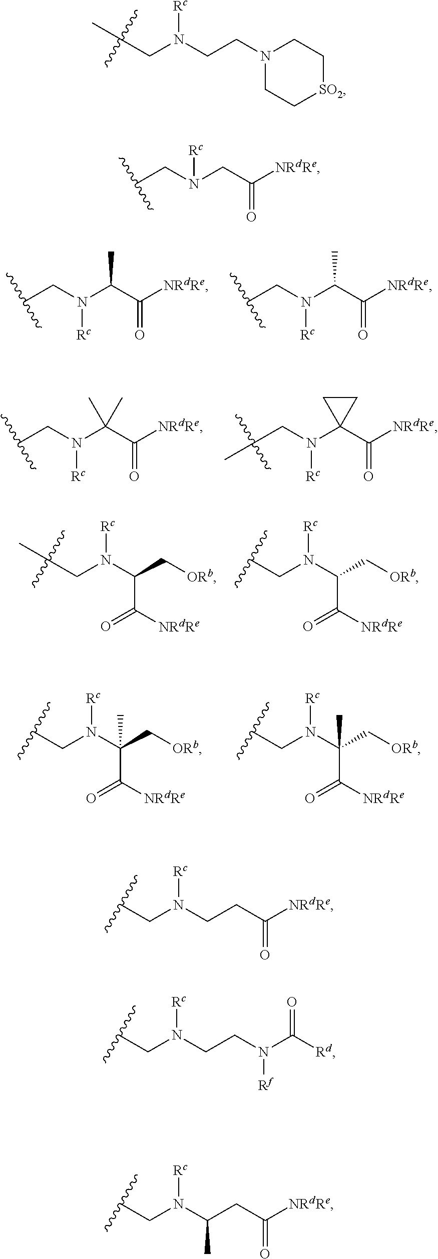 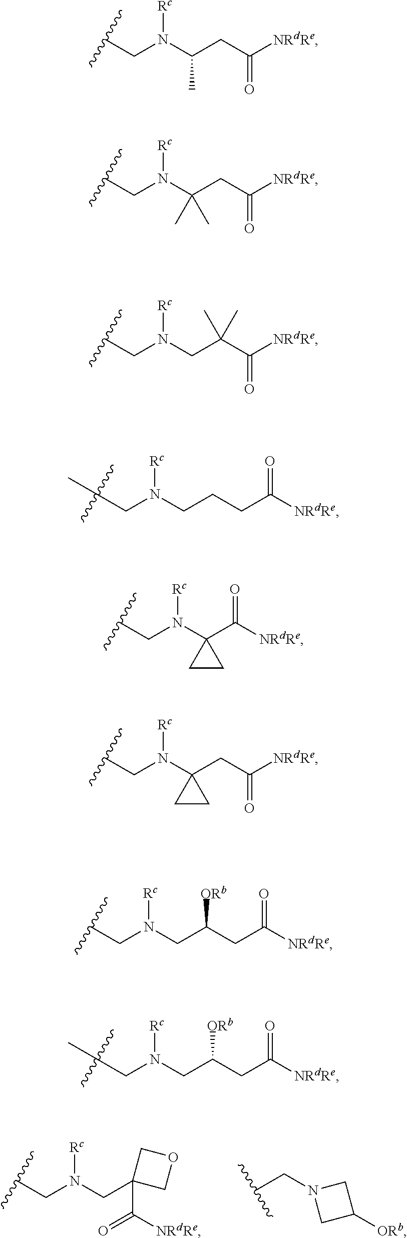 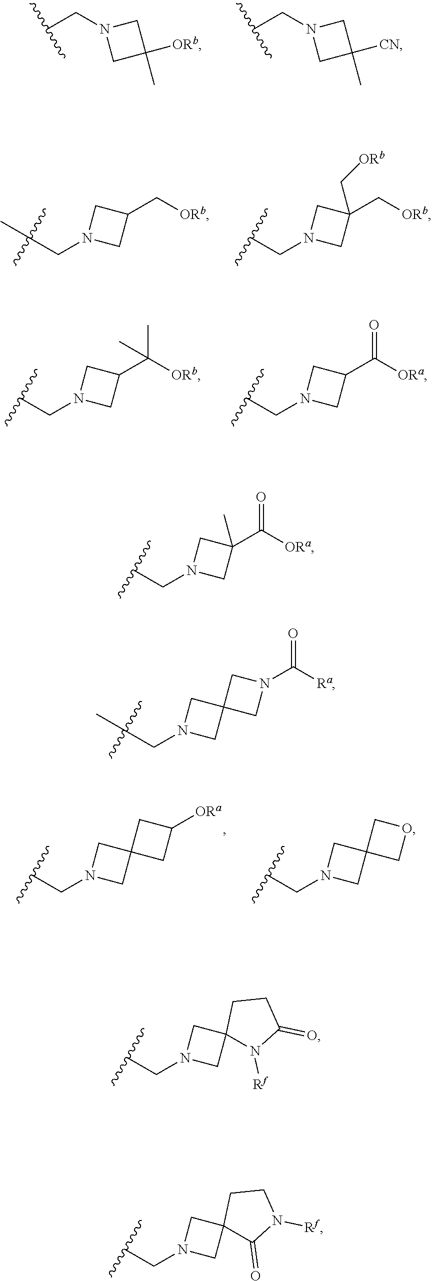 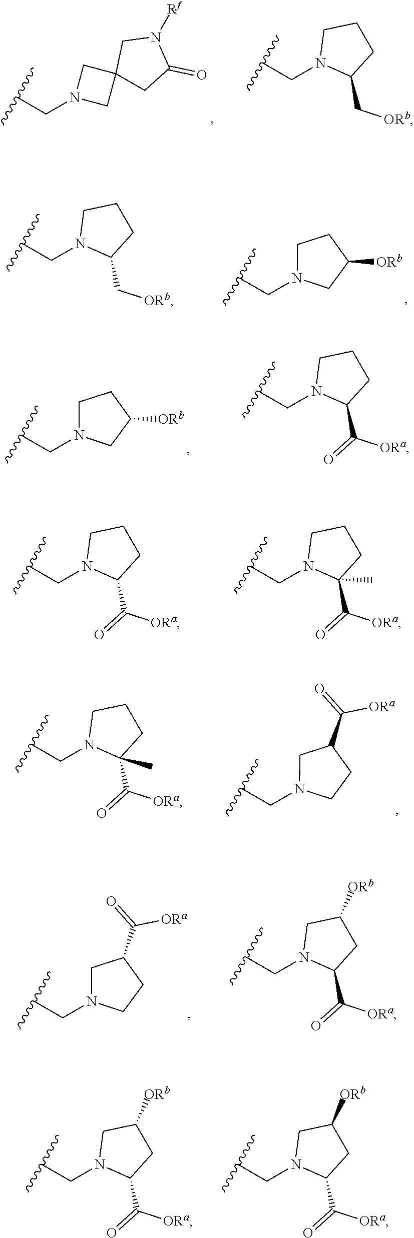 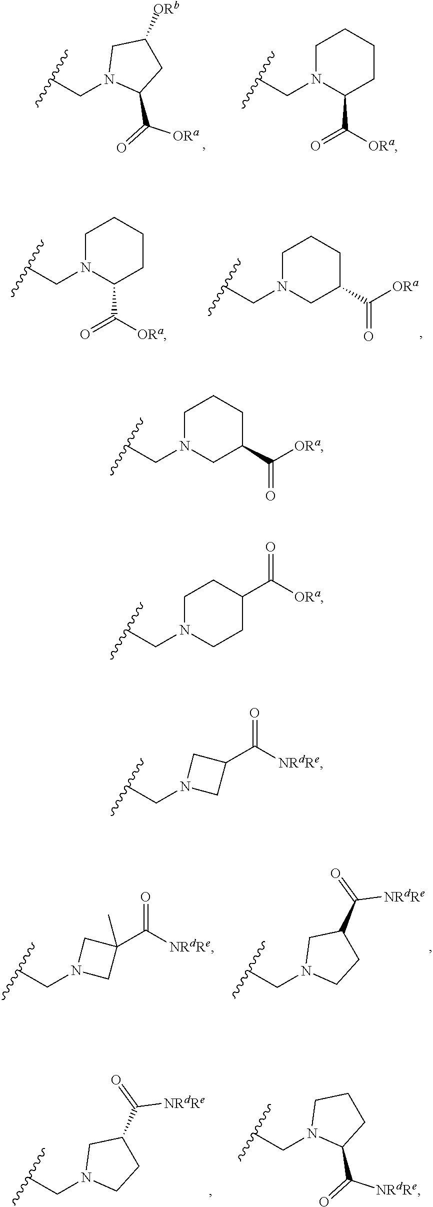 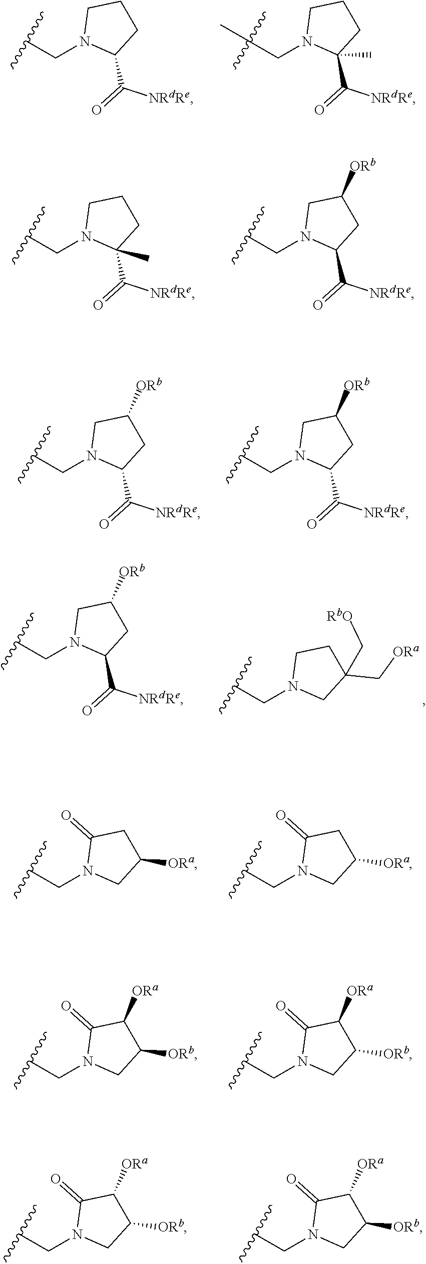 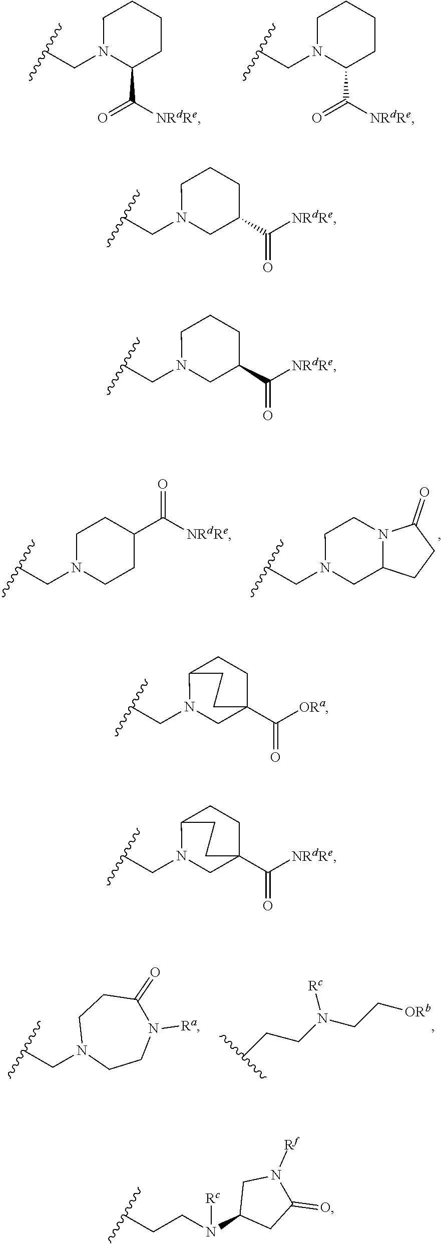 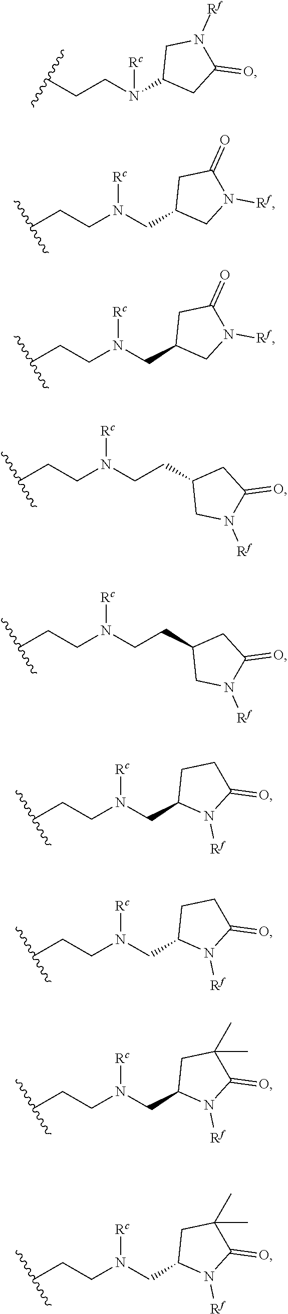 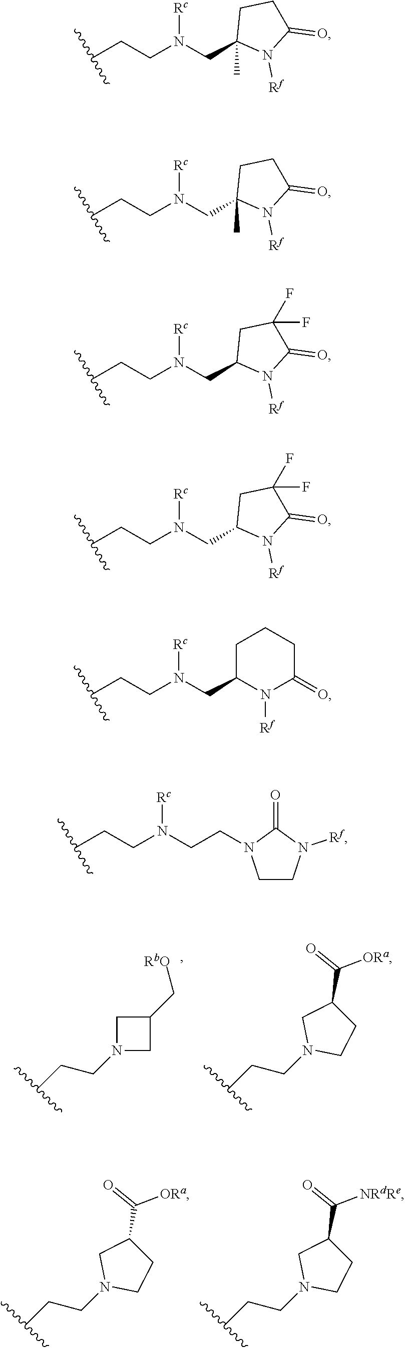 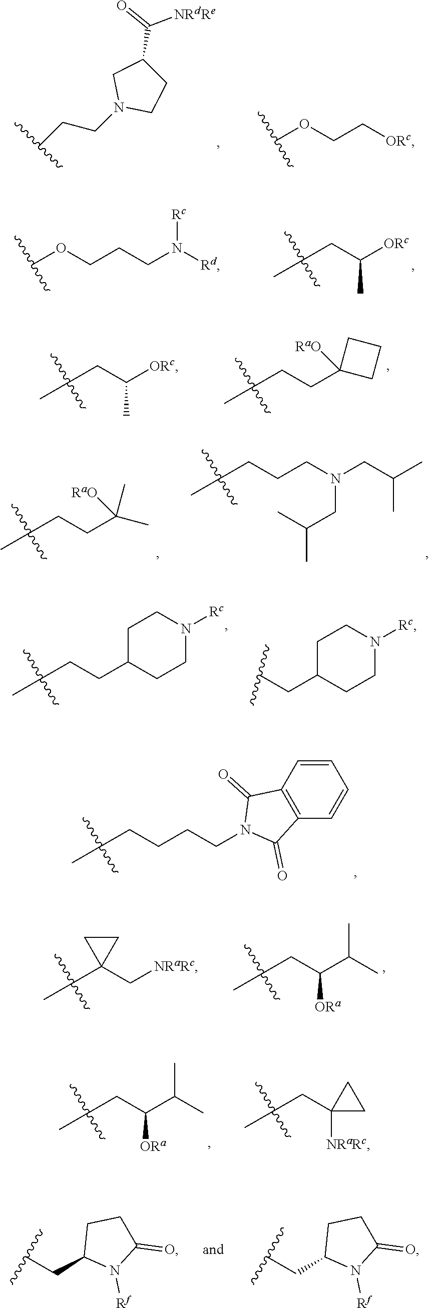
wherein:
each occurrence of R^{a}, R^{b}, R^{c}, R^{d}, R^{e}, and R^{f} is independently selected from the group consisting of H, optionally substituted C₁-C₆ alkyl, and optionally substituted C₃-C₈ cycloalkyl.

18. The compound of claim 1, which is selected from the group consisting of:
5-((4-chloro-2-(((2-hydroxyethyl)amino)methyl)-5-((3′-(3-(((2-hydroxyethyl)amino)methyl)-4-oxo-4H-pyrido[1,2-a]pyrimidin-8-yl)-2-methyl-[1,1′-biphenyl]-3-yl)methoxy)phenoxy)methyl)nicotinamide;
5-(((2-hydroxyethyl)amino)methyl)-N-(3′-(3-(((2-hydroxyethyl)amino)methyl)-4-oxo-4H-pyrido[1,2-a]pyrimidin-8-yl)-2-methyl-[1,1′-biphenyl]-3-yl)picolinamide;
5-((4-chloro-2-(((2-hydroxyethyl)amino)methyl)-5-((3′-(3-(((2-hydroxyethyl)amino)methyl)-4-oxo-4H-pyrido[1,2-a]pyrimidin-8-yl)-2,2′-dimethyl-[1,1′-biphenyl]-3-yl)methoxy)phenoxy)methyl)nicotinonitrile;
5-((4-chloro-2-(((2-hydroxyethyl)amino)methyl)-5-((3′-(3-(((2-hydroxyethyl)amino)methyl)-4-oxo-4H-pyrido[1,2-a]pyrimidin-8-yl)-2-methyl-[1,1′-biphenyl]-3-yl)methoxy)phenoxy)methyl)nicotinonitrile;
5-((4-chloro-2-(((2-hydroxyethyl)amino)methyl)-5-((3′-(7-(((2-hydroxyethyl)amino)methyl)-4-oxoquinazolin-3(4H)-yl)-2-methyl-[1,1′-biphenyl]-3-yl)methoxy)phenoxy)methyl)nicotinonitrile;
5-((4-chloro-2-(((2-hydroxyethyl)amino)methyl)-5-((3′-(7-(((2-hydroxyethyl)amino)methyl)-4-oxoquinazolin-3(4H)-yl)-2,2′-dimethyl-[1,1′-biphenyl]-3-yl)methoxy)phenoxy)methyl)nicotinonitrile;
(S)-2-(((8-(3′-((4-((((S)-2-carboxy-1-hydroxypropan-2-yl)amino)methyl)-2-chloro-5-((5-cyanopyridin-3-yl)methoxy)phenoxy)methyl)-2,2′-dimethyl-[1,1′-biphenyl]-3-yl)-4-oxo-4H-pyrido[1,2-a]pyrimidin-3-yl)methyl)amino)-3-hydroxy-2-methylpropanoic acid;
(S)-2-(((8-(3′-((4-((((R)-2-carboxy-1-hydroxypropan-2-yl)amino)methyl)-2-chloro-5-((5-cyanopyridin-3-yl)methoxy)phenoxy)methyl)-2,2′-dimethyl-[1,1′-biphenyl]-3-yl)-4-oxo-4H-pyrido[1,2-a]pyrimidin-3-yl)methyl)amino)-3-hydroxy-2-methylpropanoic acid;
(R)-2-(((8-(3′-((4-((((R)-2-carboxy-1-hydroxypropan-2-yl)amino)methyl)-2-chloro-5-((5-cyanopyridin-3-yl)methoxy)phenoxy)methyl)-2,2′-dimethyl-[1,1′-biphenyl]-3-yl)-4-oxo-4H-pyrido[1,2-a]pyrimidin-3-yl)methyl)amino)-3-hydroxy-2-methylpropanoic acid;
(R)-2-(((8-(3′-((4-((((R)-2-carboxy-1-hydroxypropan-2-yl)amino)methyl)-2-chloro-5-((5-cyanopyridin-3-yl)methoxy)phenoxy)methyl)-2,2′-dimethyl-[1,1′-biphenyl]-3-yl)-4-oxo-4H-pyrido[1,2-a]pyrimidin-3-yl)methyl)amino)-3-hydroxy-2-methylpropanoic acid;
5-((4-chloro-5-((3′-(1-(2-((2-hydroxyethyl)amino)ethyl)-4-methyl-2,5-dioxo-2,3,4,5-tetrahydro-1H-benzo[e][1,4]diazepin-7-yl)-2,2′-dimethyl-[1,1′-biphenyl]-3-yl)methoxy)-2-(((2-hydroxyethyl)amino)methyl)phenoxy)methyl)nicotinonitrile;
5-((4-chloro-2-(((2-hydroxyethyl)amino)methyl)-5-((3′-(3-(((2-hydroxyethyl)amino)methyl)-4-oxo-4H-quinolizin-8-yl)-2,2′-dimethyl-[1,1′-biphenyl]-3-yl)methoxy)phenoxy)methyl)nicotinonitrile;
(S)-2-(((8-(3′-((4-((((S)-2-carboxy-1-hydroxypropan-2-yl)amino)methyl)-2-chloro-5-((5-cyanopyridin-3-yl)methoxy)phenoxy)methyl)-2,2′-dimethyl-[1,1′-biphenyl]-3-yl)-4-oxo-4H-pyrido[1,2-a]pyrimidin-3-yl)methyl)amino)-3-hydroxy-2-methylpropanoic acid;
(S)-2-(((8-(3′-((4-((((R)-2-carboxy-1-hydroxypropan-2-yl)amino)methyl)-2-chloro-5-((5-cyanopyridin-3-yl)methoxy)phenoxy)methyl)-2,2′-dimethyl-[1,1′-biphenyl]-3-yl)-4-oxo-4H-pyrido[1,2-a]pyrimidin-3-yl)methyl)amino)-3-hydroxy-2-methylpropanoic acid;
(R)-2-(((8-(3′-((4-((((S)-2-carboxy-1-hydroxypropan-2-yl)amino)methyl)-2-chloro-5-((5-cyanopyridin-3-yl)methoxy)phenoxy)methyl)-2,2′-dimethyl-[1,1′-biphenyl]-3-yl)-4-oxo-4H-pyrido[1,2-a]pyrimidin-3-yl)methyl)amino)-3-hydroxy-2-methylpropanoic acid;
(R)-2-(((8-(3′-((4-((((R)-2-carboxy-1-hydroxypropan-2-yl)amino)methyl)-2-chloro-5-((5-cyanopyridin-3-yl)methoxy)phenoxy)methyl)-2,2′-dimethyl-[1,1′-biphenyl]-3-yl)-4-oxo-4H-pyrido[1,2-a]pyrimidin-3-yl)methyl)amino)-3-hydroxy-2-methylpropanoic acid;
(S)-1-((8-(3′-((4-(((S)-2-carboxypiperidin-1-yl)methyl)-2-chloro-5-((5-cyanopyridin-3-yl)methoxy)phenoxy)methyl)-2,2′-dimethyl-[1,1′-biphenyl]-3-yl)-4-oxo-4H-pyrido[1,2-a]pyrimidin-3-yl)methyl)piperidine-2-carboxylic acid;
(S)-1-((8-(3′-((4-(((R)-2-carboxypiperidin-1-yl)methyl)-2-chloro-5-((5-cyanopyridin-3-yl)methoxy)phenoxy)methyl)-2,2′-dimethyl-[1,1′-biphenyl]-3-yl)-4-oxo-4H-pyrido[1,2-a]pyrimidin-3-yl)methyl)piperidine-2-carboxylic acid;
(R)-1-((8-(3′-((4-(((R)-2-carboxypiperidin-1-yl)methyl)-2-chloro-5-((5-cyanopyridin-3-yl)methoxy)phenoxy)methyl)-2,2′-dimethyl-[1,1′-biphenyl]-3-yl)-4-oxo-4H-pyrido[1,2-a]pyrimidin-3-yl)methyl)piperidine-2-carboxylic acid;
(R)-1-((8-(3′-((4-(((S)-2-carboxypiperidin-1-yl)methyl)-2-chloro-5-((5-cyanopyridin-3-yl)methoxy)phenoxy)methyl)-2,2′-dimethyl-[1,1′-biphenyl]-3-yl)-4-oxo-4H-pyrido[1,2-a]pyrimidin-3-yl)methyl)piperidine-2-carboxylic acid;
5-((4-chloro-5-((2,2′-dimethyl-3′-(4-oxo-3-(((((S)-5-oxopyrrolidin-2-yl)methyl)amino)methyl)-4H-pyrido[1,2-a]pyrimidin-8-yl)-[1,1′-biphenyl]-3-yl)methoxy)-2-(((((S)-5-oxopyrrolidin-2-yl)methyl)amino)methyl)phenoxy)methyl)nicotinonitrile;
5-((4-chloro-5-((2,2′-dimethyl-3′-(4-oxo-3-(((((S)-5-oxopyrrolidin-2-yl)methyl)amino)methyl)-4H-pyrido[1,2-a]pyrimidin-8-yl)-[1,1′-biphenyl]-3-yl)methoxy)-2-(((((R)-5-oxopyrrolidin-2-yl)methyl)amino)methyl)phenoxy)methyl)nicotinonitrile;
5-((4-chloro-5-((2,2′-dimethyl-3′-(4-oxo-3-(((((R)-5-oxopyrrolidin-2-yl)methyl)amino)methyl)-4H-pyrido[1,2-a]pyrimidin-8-yl)-[1,1′-biphenyl]-3-yl)methoxy)-2-(((((R)-5-oxopyrrolidin-2-yl)methyl)amino)methyl)phenoxy)methyl)nicotinonitrile;
5-((4-chloro-5-((2,2′-dimethyl-3′-(4-oxo-3-(((((R)-5-oxopyrrolidin-2-yl)methyl)amino)methyl)-4H-pyrido[1,2-a]pyrimidin-8-yl)-[1,1′-biphenyl]-3-yl)methoxy)-2-(((((S)-5-oxopyrrolidin-2-yl)methyl)amino)methyl)phenoxy)methyl)nicotinonitrile;
5-((4-chloro-5-((2-chloro-3′-(3-(((2-hydroxyethyl)amino)methyl)-4-oxo-4H-pyrido[1,2-a]pyrimidin-8-yl)-2′-methyl-[1,1′-biphenyl]-3-yl)methoxy)-2-(((2-hydroxyethyl)amino)methyl)phenoxy)methyl)nicotinonitrile;
8,8′-(2,2′-dimethyl-[1,1′-biphenyl]-3,3′-diyl)bis(3-(((2-hydroxyethyl)amino)methyl)-4H-pyrido[1,2-a]pyrimidin-4-one);
5-((4-chloro-5-((2-cyano-3′-(3-(((2-hydroxyethyl)amino)methyl)-4-oxo-4H-pyrido[1,2-a]pyrimidin-8-yl)-2′-methyl-[1,1′-biphenyl]-3-yl)methoxy)-2-(((2-hydroxyethyl)amino)methyl)phenoxy)methyl)nicotinonitrile;
5-((4-chloro-2-(((2-hydroxyethyl)amino)methyl)-5-((3′-(8-(((2-hydroxyethyl)amino)methyl)-4-oxo-4H-pyrido[1,2-a]pyrimidin-3-yl)-2,2′-dimethyl-[1,1′-biphenyl]-3-yl)methoxy)phenoxy)methyl)nicotinonitrile;
5-((4-chloro-2-(((2-hydroxyethyl)amino)methyl)-5-(((3′-(3-(((2-hydroxyethyl)amino)methyl)-4-oxo-4H-pyrido[1,2-a]pyrimidin-8-yl)-2,2′-dimethyl-[1,1′-biphenyl]-3-yl)oxy)methyl)phenoxy)methyl)nicotinonitrile;
5-((4-chloro-5-(((2,2′-dimethyl-3′-(4-oxo-3-(((((S)-5-oxopyrrolidin-2-yl)methyl)amino)methyl)-4H-pyrido[1,2-a]pyrimidin-8-yl)-[1,1′-biphenyl]-3-yl)oxy)methyl)-2-(((((S)-5-oxopyrrolidin-2-yl)methyl)amino)methyl)phenoxy)methyl)nicotinonitrile;
5-((4-chloro-5-(((2,2′-dimethyl-3′-(4-oxo-3-(((((R)-5-oxopyrrolidin-2-yl)methyl)amino)methyl)-4H-pyrido[1,2-a]pyrimidin-8-yl)-[1,1′-biphenyl]-3-yl)oxy)methyl)-2-(((((S)-5-oxopyrrolidin-2-yl)methyl)amino)methyl)phenoxy)methyl)nicotinonitrile;
5-((4-chloro-5-(((2,2′-dimethyl-3′-(4-oxo-3-(((((S)-5-oxopyrrolidin-2-yl)methyl)amino)methyl)-4H-pyrido[1,2-a]pyrimidin-8-yl)-[1,1′-biphenyl]-3-yl)oxy)methyl)-2-(((((R)-5-oxopyrrolidin-2-yl)methyl)amino)methyl)phenoxy)methyl)nicotinonitrile;
5-((4-chloro-5-(((2,2′-dimethyl-3′-(4-oxo-3-(((((R)-5-oxopyrrolidin-2-yl)methyl)amino)methyl)-4H-pyrido[1,2-a]pyrimidin-8-yl)-[1,1′-biphenyl]-3-yl)oxy)methyl)-2-(((((R)-5-oxopyrrolidin-2-yl)methyl)amino)methyl)phenoxy)methyl)nicotinonitrile;
5-((4-chloro-5-((2,2′-dimethyl-3′-(4-methyl-2,5-dioxo-1-(2-((((S)-5-oxopyrrolidin-2-yl)methyl)amino)ethyl)-2,3,4,5-tetrahydro-1H-benzo[e][1,4]diazepin-7-yl)-[1,1′-biphenyl]-3-yl)methoxy)-2-(((((S)-5-oxopyrrolidin-2-yl)methyl)amino)methyl)phenoxy)methyl)nicotinonitrile;
5-((4-chloro-5-((2,2′-dimethyl-3′-(4-methyl-2,5-dioxo-1-(2-((((R)-5-oxopyrrolidin-2-yl)methyl)amino)ethyl)-2,3,4,5-tetrahydro-1H-benzo[e][1,4]diazepin-7-yl)-[1,1′-biphenyl]-3-yl)methoxy)-2-(((((S)-5-oxopyrrolidin-2-yl)methyl)amino)methyl)phenoxy)methyl)nicotinonitrile;
5-((4-chloro-5-((2,2′-dimethyl-3′-(4-methyl-2,5-dioxo-1-(2-((((S)-5-oxopyrrolidin-2-yl)methyl)amino)ethyl)-2,3,4,5-tetrahydro-1H-benzo[e][1,4]diazepin-7-yl)-[1,1′-biphenyl]-3-yl)methoxy)-2-(((((R)-5-oxopyrrolidin-2-yl)methyl)amino)methyl)phenoxy)methyl)nicotinonitrile;
5-((4-chloro-5-((2,2′-dimethyl-3′-(4-methyl-2,5-dioxo-1-(2-((((R)-5-oxopyrrolidin-2-yl)methyl)amino)ethyl)-2,3,4,5-tetrahydro-1H-benzo[e][1,4]diazepin-7-yl)-[1,1′-biphenyl]-3-yl)methoxy)-2-(((((R)-5-oxopyrrolidin-2-yl)methyl)amino)methyl)phenoxy)methyl)nicotinonitrile;
(Z)-8-(3′-(2-fluoro-2-(5-(((2-hydroxyethyl)amino)methyl)pyridin-2-yl)vinyl)-2,2′-dimethyl-[1,1′-biphenyl]-3-yl)-3-(((2-hydroxyethyl)amino)methyl)-4H-pyrido[1,2-a]pyrimidin-4-one;
(R)-5-((4-chloro-5-((3′-(5-((2-hydroxyethyl)amino)-1-methyl-2-oxo-2,3,4,5-tetrahydro-1H-benzo[b]azepin-8-yl)-2,2′-dimethyl-[1,1′-biphenyl]-3-yl)methoxy)-2-((((2-hydroxyethyl)amino)methyl)phenoxy)methyl)nicotinonitrile;
(S)-5-((4-chloro-5-((3′-(5-((2-hydroxyethyl)amino)-1-methyl-2-oxo-2,3,4,5-tetrahydro-1H-benzo[b]azepin-8-yl)-2,2′-dimethyl-[1,1′-biphenyl]-3-yl)methoxy)-2-((((2-hydroxyethyl)amino)methyl)phenoxy)methyl)nicotinonitrile;
5-((4-chloro-2-(((2-hydroxyethyl)amino)methyl)-5-((3′-(4-(((2-hydroxyethyl)amino)methyl)-2-oxopyridin-1(2H)-yl)-2,2′-dimethyl-[1,1′-biphenyl]-3-yl)methoxy)phenoxy)methyl)nicotinonitrile;
5-((4-chloro-5-((2,2′-dimethyl-3′-(4-oxo-3-(((((S)-5-oxopyrrolidin-2-yl)methyl)amino)methyl)-4H-quinolizin-8-yl)-[1,1′-biphenyl]-3-yl)methoxy)-2-(((((S)-5-oxopyrrolidin-2-yl)methyl)amino)methyl)phenoxy)methyl)nicotinonitrile;
5-((4-chloro-5-((2,2′-dimethyl-3′-(4-oxo-3-(((((R)-5-oxopyrrolidin-2-yl)methyl)amino)methyl)-4H-quinolizin-8-yl)-[1,1′-biphenyl]-3-yl)methoxy)-2-(((((S)-5-oxopyrrolidin-2-yl)methyl)amino)methyl)phenoxy)methyl)nicotinonitrile;
5-((4-chloro-5-((2,2′-dimethyl-3′-(4-oxo-3-(((((S)-5-oxopyrrolidin-2-yl)methyl)amino)methyl)-4H-quinolizin-8-yl)-[1,1′-biphenyl]-3-yl)methoxy)-2-(((((R)-5-oxopyrrolidin-2-yl)methyl)amino)methyl)phenoxy)methyl)nicotinonitrile;
5-((4-chloro-5-((2,2′-dimethyl-3′-(4-oxo-3-(((((R)-5-oxopyrrolidin-2-yl)methyl)amino)methyl)-4H-quinolizin-8-yl)-[1,1′-biphenyl]-3-yl)methoxy)-2-(((((R)-5-oxopyrrolidin-2-yl)methyl)amino)methyl)phenoxy)methyl)nicotinonitrile;
5-((4-chloro-5-((2′-cyano-3′-(3-(((2-hydroxyethyl)amino)methyl)-4-oxo-4H-pyrido[1,2-a]pyrimidin-8-yl)-2-methyl-[1,1′-biphenyl]-3-yl)methoxy)-2-(((2-hydroxyethyl)amino)methyl)phenoxy)methyl)nicotinonitrile;
5-((4-chloro-5-((3′-(1-(2-(3-(hydroxymethyl)azetidin-1-yl)ethyl)-4-methyl-2,5-dioxo-2,3,4,5-tetrahydro-1H-benzo[e][1,4]diazepin-7-yl)-2,2′-dimethyl-[1,1′-biphenyl]-3-yl)methoxy)-2-((3-(hydroxymethyl)azetidin-1-yl)methyl)phenoxy)methyl)nicotinonitrile;
5-((4-chloro-5-(((2,2′-dimethyl-3′-(4-methyl-2,5-dioxo-1-(2-((((S)-5-oxopyrrolidin-2-yl)methyl)amino)ethyl)-2,3,4,5-tetrahydro-1H-benzo[e][1,4]diazepin-7-yl)-[1,1′-biphenyl]-3-yl)oxy)methyl)-2-(((((S)-5-oxopyrrolidin-2-yl)methyl)amino)methyl)phenoxy)methyl)nicotinonitrile;
5-((4-chloro-5-(((2,2′-dimethyl-3′-(4-methyl-2,5-dioxo-1-(2-((((R)-5-oxopyrrolidin-2-yl)methyl)amino)ethyl)-2,3,4,5-tetrahydro-1H-benzo[e][1,4]diazepin-7-yl)-[1,1′-biphenyl]-3-yl)oxy)methyl)-2-(((((S)-5-oxopyrrolidin-2-yl)methyl)amino)methyl)phenoxy)methyl)nicotinonitrile;
5-((4-chloro-5-(((2,2′-dimethyl-3′-(4-methyl-2,5-dioxo-1-(2-((((S)-5-oxopyrrolidin-2-yl)methyl)amino)ethyl)-2,3,4,5-tetrahydro-1H-benzo[e][1,4]diazepin-7-yl)-[1,1′-biphenyl]-3-yl)oxy)methyl)-2-(((((R)-5-oxopyrrolidin-2-yl)methyl)amino)methyl)phenoxy)methyl)nicotinonitrile;
5-((4-chloro-5-(((2,2′-dimethyl-3′-(4-methyl-2,5-dioxo-1-(2-((((R)-5-oxopyrrolidin-2-yl)methyl)amino)ethyl)-2,3,4,5-tetrahydro-1H-benzo[e][1,4]diazepin-7-yl)-[1,1′-biphenyl]-3-yl)oxy)methyl)-2-(((((R)-5-oxopyrrolidin-2-yl)methyl)amino)methyl)phenoxy)methyl)nicotinonitrile;
5-((4-chloro-5-((2,2′-dimethyl-3′-(4-oxo-3-(((((R)-5-oxopyrrolidin-2-yl)methyl)amino)methyl)-4H-quinolizin-8-yl)-[1,1′-biphenyl]-3-yl)methoxy)-2-(((((R)-5-oxopyrrolidin-2-yl)methyl)amino)methyl)phenoxy)methyl)nicotinonitrile;
5-((4-chloro-5-((2,2′-dimethyl-3′-(4-oxo-3-(((((R)-5-oxopyrrolidin-2-yl)methyl)amino)methyl)-4H-quinolizin-8-yl)-[1,1′-biphenyl]-3-yl)methoxy)-2-(((((S)-5-oxopyrrolidin-2-yl)methyl)amino)methyl)phenoxy)methyl)nicotinonitrile;
5-((4-chloro-5-((2,2′-dimethyl-3′-(4-oxo-3-(((((S)-5-oxopyrrolidin-2-yl)methyl)amino)methyl)-4H-quinolizin-8-yl)-[1,1′-biphenyl]-3-yl)methoxy)-2-(((((R)-5-oxopyrrolidin-2-yl)methyl)amino)methyl)phenoxy)methyl)nicotinonitrile;
5-((4-chloro-5-((2,2′-dimethyl-3′-(4-oxo-3-(((((S)-5-oxopyrrolidin-2-yl)methyl)amino)methyl)-4H-quinolizin-8-yl)-[1,1′-biphenyl]-3-yl)methoxy)-2-(((((S)-5-oxopyrrolidin-2-yl)methyl)amino)methyl)phenoxy)methyl)nicotinonitrile;
1-((8-(3′-((4-((3-carboxyazetidin-1-yl)methyl)-2-chloro-5-((5-cyanopyridin-3-yl)methoxy)phenoxy)methyl)-2,2′-dimethyl-[1,1′-biphenyl]-3-yl)-4-oxo-4H-quinolizin-3-yl)methyl)azetidine-3-carboxylic acid;
(S)-1-(4-((3′-(1-(2-((S)-3-carboxypyrrolidin-1-yl)ethyl)-4-methyl-2,5-dioxo-2,3,4,5-tetrahydro-1H-benzo[e][1,4]diazepin-7-yl)-2,2′-dimethyl-[1,1′-biphenyl]-3-yl)methoxy)-5-chloro-2-((5-cyanopyridin-3-yl)methoxy)benzyl)pyrrolidine-3-carboxylic acid;
(R)-1-(4-((3′-(1-(2-((S)-3-carboxypyrrolidin-1-yl)ethyl)-4-methyl-2,5-dioxo-2,3,4,5-tetrahydro-1H-benzo[e][1,4]diazepin-7-yl)-2,2′-dimethyl-[1,1′-biphenyl]-3-yl)methoxy)-5-chloro-2-((5-cyanopyridin-3-yl)methoxy)benzyl)pyrrolidine-3-carboxylic acid;
(S)-1-(4-((3′-(1-(2-((R)-3-carboxypyrrolidin-1-yl)ethyl)-4-methyl-2,5-dioxo-2,3,4,5-tetrahydro-1H-benzo[e][1,4]diazepin-7-yl)-2,2′-dimethyl-[1,1′-biphenyl]-3-yl)methoxy)-5-chloro-2-((5-cyanopyridin-3-yl)methoxy)benzyl)pyrrolidine-3-carboxylic acid;
(R)-1-(4-((3′-(1-(2-((R)-3-carboxypyrrolidin-1-yl)ethyl)-4-methyl-2,5-dioxo-2,3,4,5-tetrahydro-1H-benzo[e][1,4]diazepin-7-yl)-2,2′-dimethyl-[1,1′-biphenyl]-3-yl)methoxy)-5-chloro-2-((5-cyanopyridin-3-yl)methoxy)benzyl)pyrrolidine-3-carboxylic acid;
8-(3′-(6-methoxy-5-(((((S)-5-oxopyrrolidin-2-yl)methyl)amino)methyl)pyridin-2-yl)-2,2′-dimethyl-[1,1′-biphenyl]-3-yl)-3-(((((S)-5-oxopyrrolidin-2-yl)methyl)amino)methyl)-4H-pyrido[1,2-a]pyrimidin-4-one;
8-(3′-(6-methoxy-5-(((((R)-5-oxopyrrolidin-2-yl)methyl)amino)methyl)pyridin-2-yl)-2,2′-dimethyl-[1,1′-biphenyl]-3-yl)-3-(((((S)-5-oxopyrrolidin-2-yl)methyl)amino)methyl)-4H-pyrido[1,2-a]pyrimidin-4-one;
8-(3′-(6-methoxy-5-(((((S)-5-oxopyrrolidin-2-yl)methyl)amino)methyl)pyridin-2-yl)-2,2′-dimethyl-[1,1′-biphenyl]-3-yl)-3-(((((R)-5-oxopyrrolidin-2-yl)methyl)amino)methyl)-4H-pyrido[1,2-a]pyrimidin-4-one;
8-(3′-(6-methoxy-5-(((((R)-5-oxopyrrolidin-2-yl)methyl)amino)methyl)pyridin-2-yl)-2,2′-dimethyl-[1,1′-biphenyl]-3-yl)-3-(((((R)-5-oxopyrrolidin-2-yl)methyl)amino)methyl)-4H-pyrido[1,2-a]pyrimidin-4-one;
8-(2′-chloro-3′-(6-methoxy-5-(((((S)-5-oxopyrrolidin-2-yl)methyl)amino)methyl)pyridin-2-yl)-2-methyl-[1,1′-biphenyl]-3-yl)-3-(((((S)-5-oxopyrrolidin-2-yl)methyl)amino)methyl)-4H-pyrido[1,2-a]pyrimidin-4-one;
8-(2′-chloro-3′-(6-methoxy-5-(((((R)-5-oxopyrrolidin-2-yl)methyl)amino)methyl)pyridin-2-yl)-2-methyl-[1,1′-biphenyl]-3-yl)-3-(((((S)-5-oxopyrrolidin-2-yl)methyl)amino)methyl)-4H-pyrido[1,2-a]pyrimidin-4-one;
8-(2′-chloro-3′-(6-methoxy-5-(((((S)-5-oxopyrrolidin-2-yl)methyl)amino)methyl)pyridin-2-yl)-2-methyl-[1,1′-biphenyl]-3-yl)-3-(((((R)-5-oxopyrrolidin-2-yl)methyl)amino)methyl)-4H-pyrido[1,2-a]pyrimidin-4-one;
8-(2′-chloro-3′-(6-methoxy-5-(((((R)-5-oxopyrrolidin-2-yl)methyl)amino)methyl)pyridin-2-yl)-2-methyl-[1,1′-biphenyl]-3-yl)-3-(((((R)-5-oxopyrrolidin-2-yl)methyl)amino)methyl)-4H-pyrido[1,2-a]pyrimidin-4-one;
8-(2,2′-dichloro-3′-(6-methoxy-5-(((((S)-5-oxopyrrolidin-2-yl)methyl)amino)methyl)pyridin-2-yl)-[1,1′-biphenyl]-3-yl)-3-(((((S)-5-oxopyrrolidin-2-yl)methyl)amino)methyl)-4H-pyrido[1,2-a]pyrimidin-4-one;
8-(2,2′-dichloro-3′-(6-methoxy-5-(((((R)-5-oxopyrrolidin-2-yl)methyl)amino)methyl)pyridin-2-yl)-[1,1′-biphenyl]-3-yl)-3-(((((S)-5-oxopyrrolidin-2-yl)methyl)amino)methyl)-4H-pyrido[1,2-a]pyrimidin-4-one;
8-(2,2′-dichloro-3′-(6-methoxy-5-(((((S)-5-oxopyrrolidin-2-yl)methyl)amino)methyl)pyridin-2-yl)-[1,1′-biphenyl]-3-yl)-3-(((((R)-5-oxopyrrolidin-2-yl)methyl)amino)methyl)-4H-pyrido[1,2-a]pyrimidin-4-one;
8-(2,2′-dichloro-3′-(6-methoxy-5-(((((R)-5-oxopyrrolidin-2-yl)methyl)amino)methyl)pyridin-2-yl)-[1,1′-biphenyl]-3-yl)-3-(((((R)-5-oxopyrrolidin-2-yl)methyl)amino)methyl)-4H-pyrido[1,2-a]pyrimidin-4-one;
2-(((S)-7-(2,2′-dichloro-3′-((R)-5-((2-hydroxyethyl)amino)-8-methyl-5,6,7,8-tetrahydro-1,8-naphthyridin-2-yl)-[1,1′-biphenyl]-3-yl)-1-methyl-1,2,3,4-tetrahydro-1,8-naphthyridin-4-yl)amino)ethan-1-ol;
2-(((S)-7-(2,2′-dichloro-3′-((S)-5-((2-hydroxyethyl)amino)-8-methyl-5,6,7,8-tetrahydro-1,8-naphthyridin-2-yl)-[1,1′-biphenyl]-3-yl)-1-methyl-1,2,3,4-tetrahydro-1,8-naphthyridin-4-yl)amino)ethan-1-ol;
2-(((R)-7-(2,2′-dichloro-3′-((R)-5-((2-hydroxyethyl)amino)-8-methyl-5,6,7,8-tetrahydro-1,8-naphthyridin-2-yl)-[1,1′-biphenyl]-3-yl)-1-methyl-1,2,3,4-tetrahydro-1,8-naphthyridin-4-yl)amino)ethan-1-ol;
2-(((R)-7-(2,2′-dichloro-3′-((S)-5-((2-hydroxyethyl)amino)-8-methyl-5,6,7,8-tetrahydro-1,8-naphthyridin-2-yl)-[1,1′-biphenyl]-3-yl)-1-methyl-1,2,3,4-tetrahydro-1,8-naphthyridin-4-yl)amino)ethan-1-ol;
2,2′-(2,2′-dichloro-[1,1′-biphenyl]-3,3′-diyl)bis(9-methyl-7-(3-(methylamino)propoxy)-4H-pyrido[1,2-a]pyrimidin-4-one);
8-(2′-chloro-3′-(6-methoxy-5-(((((S)-5-oxopyrrolidin-2-yl)methyl)amino)methyl)pyrazin-2-yl)-2-methyl-[1,1′-biphenyl]-3-yl)-3-(((((S)-5-oxopyrrolidin-2-yl)methyl)amino)methyl)-4H-pyrido[1,2-a]pyrimidin-4-one;
8-(2′-chloro-3′-(6-methoxy-5-(((((R)-5-oxopyrrolidin-2-yl)methyl)amino)methyl)pyrazin-2-yl)-2-methyl-[1,1′-biphenyl]-3-yl)-3-(((((S)-5-oxopyrrolidin-2-yl)methyl)amino)methyl)-4H-pyrido[1,2-a]pyrimidin-4-one;
8-(2′-chloro-3′-(6-methoxy-5-(((((S)-5-oxopyrrolidin-2-yl)methyl)amino)methyl)pyrazin-2-yl)-2-methyl-[1,1′-biphenyl]-3-yl)-3-(((((R)-5-oxopyrrolidin-2-yl)methyl)amino)methyl)-4H-pyrido[1,2-a]pyrimidin-4-one;
8-(2′-chloro-3′-(6-methoxy-5-(((((R)-5-oxopyrrolidin-2-yl)methyl)amino)methyl)pyrazin-2-yl)-2-methyl-[1,1′-biphenyl]-3-yl)-3-(((((R)-5-oxopyrrolidin-2-yl)methyl)amino)methyl)-4H-pyrido[1,2-a]pyrimidin-4-one;
8-(2,2′-dimethyl-3′-(4-oxo-3-(((((R)-5-oxopyrrolidin-2-yl)methyl)amino)methyl)-4H-pyrido[1,2-a]pyrimidin-8-yl)-[1,1′-biphenyl]-3-yl)-3-(((((S)-5-oxopyrrolidin-2-yl)methyl)amino)methyl)-4H-pyrido[1,2-a]pyrimidin-4-one;
8-(2,2′-dimethyl-3′-(4-oxo-3-(((((S)-5-oxopyrrolidin-2-yl)methyl)amino)methyl)-4H-pyrido[1,2-a]pyrimidin-8-yl)-[1,1′-biphenyl]-3-yl)-3-(((((R)-5-oxopyrrolidin-2-yl)methyl)amino)methyl)-4H-pyrido[1,2-a]pyrimidin-4-one;
8-(2,2′-dimethyl-3′-(4-oxo-3-(((((R)-5-oxopyrrolidin-2-yl)methyl)amino)methyl)-4H-pyrido[1,2-a]pyrimidin-8-yl)-[1,1′-biphenyl]-3-yl)-3-(((((R)-5-oxopyrrolidin-2-yl)methyl)amino)methyl)-4H-pyrido[1,2-a]pyrimidin-4-one;
8-(2,2′-dimethyl-3′-(4-oxo-3-(((((S)-5-oxopyrrolidin-2-yl)methyl)amino)methyl)-4H-pyrido[1,2-a]pyrimidin-8-yl)-[1,1′-biphenyl]-3-yl)-3-(((((S)-5-oxopyrrolidin-2-yl)methyl)amino)methyl)-4H-pyrido[1,2-a]pyrimidin-4-one;
N,N′-((((2,2′-dichloro-[1,1′-biphenyl]-3,3′-diyl)bis(3-methoxypyrazine-5,2-diyl))bis(azanediyl))bis(ethane-2,1-diyl))diacetamide;
N,N′-((((2,2′-dichloro-[1,1′-biphenyl]-3,3′-diyl)bis(6-methoxypyrazine-5,2-diyl))bis(azanediyl))bis(ethane-2,1-diyl))diacetamide;
8-(3′-((2R)-amino-2,3-dihydro-1H-inden-5-yl)-2′-chloro-2-methyl-[1,1′-biphenyl]-3-yl)-3-(((((R)-5-oxopyrrolidin-2-yl)methyl)amino)methyl)-4H-pyrido[1,2-a]pyrimidin-4-one;
8-(3′-((2R)-amino-2,3-dihydro-1H-inden-5-yl)-2′-chloro-2-methyl-[1,1′-biphenyl]-3-yl)-3-(((((S)-5-oxopyrrolidin-2-yl)methyl)amino)methyl)-4H-pyrido[1,2-a]pyrimidin-4-one;
8-(3′-((2S)-amino-2,3-dihydro-1H-inden-5-yl)-2′-chloro-2-methyl-[1,1′-biphenyl]-3-yl)-3-(((((R)-5-oxopyrrolidin-2-yl)methyl)amino)methyl)-4H-pyrido[1,2-a]pyrimidin-4-one;
8-(3′-((2S)-amino-2,3-dihydro-1H-inden-5-yl)-2′-chloro-2-methyl-[1,1′-biphenyl]-3-yl)-3-(((((S)-5-oxopyrrolidin-2-yl)methyl)amino)methyl)-4H-pyrido[1,2-a]pyrimidin-4-one;
8-(3′-((2R)-amino-2,3-dihydro-1H-inden-5-yl)-2,2′-dimethyl-[1,1′-biphenyl]-3-yl)-3-(((((R)-5-oxopyrrolidin-2-yl)methyl)amino)methyl)-4H-pyrido[1,2-a]pyrimidin-4-one;
8-(3′-((2R)-amino-2,3-dihydro-1H-inden-5-yl)-2,2′-dimethyl-[1,1′-biphenyl]-3-yl)-3-(((((S)-5-oxopyrrolidin-2-yl)methyl)amino)methyl)-4H-pyrido[1,2-a]pyrimidin-4-one;
8-(3′-((2S)-amino-2,3-dihydro-1H-inden-5-yl)-2,2′-dimethyl-[1,1′-biphenyl]-3-yl)-3-(((((R)-5-oxopyrrolidin-2-yl)methyl)amino)methyl)-4H-pyrido[1,2-a]pyrimidin-4-one;
8-(3′-((2S)-amino-2,3-dihydro-1H-inden-5-yl)-2,2′-dimethyl-[1,1′-biphenyl]-3-yl)-3-(((((S)-5-oxopyrrolidin-2-yl)methyl)amino)methyl)-4H-pyrido[1,2-a]pyrimidin-4-one;
(5S,5'S)-5,5′-((((4S,4'S)-(2,2′-dichloro-[1,1′-biphenyl]-3,3′-diyl)bis(1-methyl-1,2,3,4-tetrahydro-1,8-naphthyridine-7,4-diyl))bis(azanediyl))bis(methylene))bis(pyrrolidin-2-one);
(5S,5'S)-5,5′-((((4R,4'S)-(2,2′-dichloro-[1,1′-biphenyl]-3,3′-diyl)bis(1-methyl-1,2,3,4-tetrahydro-1,8-naphthyridine-7,4-diyl))bis(azanediyl))bis(methylene))bis(pyrrolidin-2-one);
(5S,5'S)-5,5′-((((4S,4′R)-(2,2′-dichloro-[1,1′-biphenyl]-3,3′-diyl)bis(1-methyl-1,2,3,4-tetrahydro-1,8-naphthyridine-7,4-diyl))bis(azanediyl))bis(methylene))bis(pyrrolidin-2-one);
(5S,5'S)-5,5′-((((4R,4′R)-(2,2′-dichloro-[1,1′-biphenyl]-3,3′-diyl)bis(1-methyl-1,2,3,4-tetrahydro-1,8-naphthyridine-7,4-diyl))bis(azanediyl))bis(methylene))bis(pyrrolidin-2-one);
(5R,5'S)-5,5′-((((4S,4'S)-(2,2′-dichloro-[1,1′-biphenyl]-3,3′-diyl)bis(1-methyl-1,2,3,4-tetrahydro-1,8-naphthyridine-7,4-diyl))bis(azanediyl))bis(methylene))bis(pyrrolidin-2-one);
(5R,5'S)-5,5′-((((4R,4'S)-(2,2′-dichloro-[1,1′-biphenyl]-3,3′-diyl)bis(1-methyl-1,2,3,4-tetrahydro-1,8-naphthyridine-7,4-diyl))bis(azanediyl))bis(methylene))bis(pyrrolidin-2-one);
(5R,5'S)-5,5′-((((4S,4′R)-(2,2′-dichloro-[1,1′-biphenyl]-3,3′-diyl)bis(1-methyl-1,2,3,4-tetrahydro-1,8-naphthyridine-7,4-diyl))bis(azanediyl))bis(methylene))bis(pyrrolidin-2-one);
(5R,5'S)-5,5′-((((4R,4′R)-(2,2′-dichloro-[1,1′-biphenyl]-3,3′-diyl)bis(1-methyl-1,2,3,4-tetrahydro-1,8-naphthyridine-7,4-diyl))bis(azanediyl))bis(methylene))bis(pyrrolidin-2-one);
(5S,5′R)-5,5′-((((4S,4'S)-(2,2′-dichloro-[1,1′-biphenyl]-3,3′-diyl)bis(1-methyl-1,2,3,4-tetrahydro-1,8-naphthyridine-7,4-diyl))bis(azanediyl))bis(methylene))bis(pyrrolidin-2-one);
(5S,5′R)-5,5′-((((4R,4'S)-(2,2′-dichloro-[1,1′-biphenyl]-3,3′-diyl)bis(1-methyl-1,2,3,4-tetrahydro-1,8-naphthyridine-7,4-diyl))bis(azanediyl))bis(methylene))bis(pyrrolidin-2-one);
(5S,5′R)-5,5′-((((4S,4′R)-(2,2′-dichloro-[1,1′-biphenyl]-3,3′-diyl)bis(1-methyl-1,2,3,4-tetrahydro-1,8-naphthyridine-7,4-diyl))bis(azanediyl))bis(methylene))bis(pyrrolidin-2-one);
(5S,5′R)-5,5′-((((4R,4′R)-(2,2′-dichloro-[1,1′-biphenyl]-3,3′-diyl)bis(1-methyl-1,2,3,4-tetrahydro-1,8-naphthyridine-7,4-diyl))bis(azanediyl))bis(methylene))bis(pyrrolidin-2-one);
(5R,5′R)-5,5′-((((4S,4'S)-(2,2′-dichloro-[1,1′-biphenyl]-3,3′-diyl)bis(1-methyl-1,2,3,4-tetrahydro-1,8-naphthyridine-7,4-diyl))bis(azanediyl))bis(methylene))bis(pyrrolidin-2-one);
(5R,5′R)-5,5′-((((4R,4'S)-(2,2′-dichloro-[1,1′-biphenyl]-3,3′-diyl)bis(1-methyl-1,2,3,4-tetrahydro-1,8-naphthyridine-7,4-diyl))bis(azanediyl))bis(methylene))bis(pyrrolidin-2-one);
(5R,5′R)-5,5′-((((4S,4′R)-(2,2′-dichloro-[1,1′-biphenyl]-3,3′-diyl)bis(1-methyl-1,2,3,4-tetrahydro-1,8-naphthyridine-7,4-diyl))bis(azanediyl))bis(methylene))bis(pyrrolidin-2-one);
(5R,5′R)-5,5′-((((4R,4′R)-(2,2′-dichloro-[1,1′-biphenyl]-3,3′-diyl)bis(1-methyl-1,2,3,4-tetrahydro-1,8-naphthyridine-7,4-diyl))bis(azanediyl))bis(methylene))bis(pyrrolidin-2-one);
2-amino-N-(5-(2-chloro-2′-methyl-3′-(4-oxo-3-(((((S)-5-oxopyrrolidin-2-yl)methyl)amino)methyl)-4H-pyrido[1,2-a]pyrimidin-8-yl)-[1,1′-biphenyl]-3-yl)-2,3-dihydro-1H-inden-(2S)-yl)acetamide;
2-amino-N-(5-(2-chloro-2′-methyl-3′-(4-oxo-3-(((((R)-5-oxopyrrolidin-2-yl)methyl)amino)methyl)-4H-pyrido[1,2-a]pyrimidin-8-yl)-[1,1′-biphenyl]-3-yl)-2,3-dihydro-1H-inden-(2S)-yl)acetamide;
2-amino-N-(5-(2-chloro-2′-methyl-3′-(4-oxo-3-(((((S)-5-oxopyrrolidin-2-yl)methyl)amino)methyl)-4H-pyrido[1,2-a]pyrimidin-8-yl)-[1,1′-biphenyl]-3-yl)-2,3-dihydro-1H-inden-(2R)-yl)acetamide;
2-amino-N-(5-(2-chloro-2′-methyl-3′-(4-oxo-3-(((((R)-5-oxopyrrolidin-2-yl)methyl)amino)methyl)-4H-pyrido[1,2-a]pyrimidin-8-yl)-[1,1′-biphenyl]-3-yl)-2,3-dihydro-1H-inden-(2R)-yl)acetamide;
(S)-5-((((S)-6-(2,2′-dichloro-3′-((S)-3-((((S)-5-oxopyrrolidin-2-yl)methyl)amino)-2,3-dihydrofuro[2,3-b]pyridin-6-yl)-[1,1′-biphenyl]-3-yl)-2,3-dihydrofuro[2,3-b]pyridin-3-yl)amino)methyl)pyrrolidin-2-one;
(S)-5-((((S)-6-(2,2′-dichloro-3′-((R)-3-((((S)-5-oxopyrrolidin-2-yl)methyl)amino)-2,3-dihydrofuro[2,3-b]pyridin-6-yl)-[1,1′-biphenyl]-3-yl)-2,3-dihydrofuro[2,3-b]pyridin-3-yl)amino)methyl)pyrrolidin-2-one;
(S)-5-((((S)-6-(2,2′-dichloro-3′-((S)-3-((((R)-5-oxopyrrolidin-2-yl)methyl)amino)-2,3-dihydrofuro[2,3-b]pyridin-6-yl)-[1,1′-biphenyl]-3-yl)-2,3-dihydrofuro[2,3-b]pyridin-3-yl)amino)methyl)pyrrolidin-2-one;
(S)-5-((((S)-6-(2,2′-dichloro-3′-((R)-3-((((R)-5-oxopyrrolidin-2-yl)methyl)amino)-2,3-dihydrofuro[2,3-b]pyridin-6-yl)-[1,1′-biphenyl]-3-yl)-2,3-dihydrofuro[2,3-b]pyridin-3-yl)amino)methyl)pyrrolidin-2-one;
(R)-5-((((S)-6-(2,2′-dichloro-3′-((S)-3-((((S)-5-oxopyrrolidin-2-yl)methyl)amino)-2,3-dihydrofuro[2,3-b]pyridin-6-yl)-[1,1′-biphenyl]-3-yl)-2,3-dihydrofuro[2,3-b]pyridin-3-yl)amino)methyl)pyrrolidin-2-one;
(R)-5-((((S)-6-(2,2′-dichloro-3′-((R)-3-((((S)-5-oxopyrrolidin-2-yl)methyl)amino)-2,3-dihydrofuro[2,3-b]pyridin-6-yl)-[1,1′-biphenyl]-3-yl)-2,3-dihydrofuro[2,3-b]pyridin-3-yl)amino)methyl)pyrrolidin-2-one;
(R)-5-((((S)-6-(2,2′-dichloro-3′-((S)-3-((((R)-5-oxopyrrolidin-2-yl)methyl)amino)-2,3-dihydrofuro[2,3-b]pyridin-6-yl)-[1,1′-biphenyl]-3-yl)-2,3-dihydrofuro[2,3-b]pyridin-3-yl)amino)methyl)pyrrolidin-2-one;
(R)-5-((((S)-6-(2,2′-dichloro-3′-((R)-3-((((R)-5-oxopyrrolidin-2-yl)methyl)amino)-2,3-dihydrofuro[2,3-b]pyridin-6-yl)-[1,1′-biphenyl]-3-yl)-2,3-dihydrofuro[2,3-b]pyridin-3-yl)amino)methyl)pyrrolidin-2-one;
(S)-5-((((R)-6-(2,2′-dichloro-3′-((S)-3-((((S)-5-oxopyrrolidin-2-yl)methyl)amino)-2,3-dihydrofuro[2,3-b]pyridin-6-yl)-[1,1′-biphenyl]-3-yl)-2,3-dihydrofuro[2,3-b]pyridin-3-yl)amino)methyl)pyrrolidin-2-one;
(S)-5-((((R)-6-(2,2′-dichloro-3′-((R)-3-((((S)-5-oxopyrrolidin-2-yl)methyl)amino)-2,3-dihydrofuro[2,3-b]pyridin-6-yl)-[1,1′-biphenyl]-3-yl)-2,3-dihydrofuro[2,3-b]pyridin-3-yl)amino)methyl)pyrrolidin-2-one;
(S)-5-((((R)-6-(2,2′-dichloro-3′-((S)-3-((((R)-5-oxopyrrolidin-2-yl)methyl)amino)-2,3-dihydrofuro[2,3-b]pyridin-6-yl)-[1,1′-biphenyl]-3-yl)-2,3-dihydrofuro[2,3-b]pyridin-3-yl)amino)methyl)pyrrolidin-2-one;
(S)-5-((((R)-6-(2,2′-dichloro-3′-((R)-3-((((R)-5-oxopyrrolidin-2-yl)methyl)amino)-2,3-dihydrofuro[2,3-b]pyridin-6-yl)-[1,1′-biphenyl]-3-yl)-2,3-dihydrofuro[2,3-b]pyridin-3-yl)amino)methyl)pyrrolidin-2-one;
(R)-5-((((R)-6-(2,2′-dichloro-3′-((S)-3-((((S)-5-oxopyrrolidin-2-yl)methyl)amino)-2,3-dihydrofuro[2,3-b]pyridin-6-yl)-[1,1′-biphenyl]-3-yl)-2,3-dihydrofuro[2,3-b]pyridin-3-yl)amino)methyl)pyrrolidin-2-one;
(R)-5-((((R)-6-(2,2′-dichloro-3′-((R)-3-((((S)-5-oxopyrrolidin-2-yl)methyl)amino)-2,3-dihydrofuro[2,3-b]pyridin-6-yl)-[1,1′-biphenyl]-3-yl)-2,3-dihydrofuro[2,3-b]pyridin-3-yl)amino)methyl)pyrrolidin-2-one;
(R)-5-((((R)-6-(2,2′-dichloro-3′-((S)-3-((((R)-5-oxopyrrolidin-2-yl)methyl)amino)-2,3-dihydrofuro[2,3-b]pyridin-6-yl)-[1,1′-biphenyl]-3-yl)-2,3-dihydrofuro[2,3-b]pyridin-3-yl)amino)methyl)pyrrolidin-2-one;
(R)-5-((((R)-6-(2,2′-dichloro-3′-((R)-3-((((R)-5-oxopyrrolidin-2-yl)methyl)amino)-2,3-dihydrofuro[2,3-b]pyridin-6-yl)-[1,1′-biphenyl]-3-yl)-2,3-dihydrofuro[2,3-b]pyridin-3-yl)amino)methyl)pyrrolidin-2-one;
(S)-2-amino-N—((S)-5-(2-chloro-2′-methyl-3′-(4-oxo-3-(((((S)-5-oxopyrrolidin-2-yl)methyl)amino)methyl)-4H-pyrido[1,2-a]pyrimidin-8-yl)-[1,1′-biphenyl]-3-yl)-2,3-dihydro-1H-inden-2-yl)-3-hydroxypropanamide;
(S)-2-amino-N—((R)-5-(2-chloro-2′-methyl-3′-(4-oxo-3-(((((S)-5-oxopyrrolidin-2-yl)methyl)amino)methyl)-4H-pyrido[1,2-a]pyrimidin-8-yl)-[1,1′-biphenyl]-3-yl)-2,3-dihydro-1H-inden-2-yl)-3-hydroxypropanamide;
(S)-2-amino-N—((S)-5-(2-chloro-2′-methyl-3′-(4-oxo-3-(((((R)-5-oxopyrrolidin-2-yl)methyl)amino)methyl)-4H-pyrido[1,2-a]pyrimidin-8-yl)-[1,1′-biphenyl]-3-yl)-2,3-dihydro-1H-inden-2-yl)-3-hydroxypropanamide;
(S)-2-amino-N—((R)-5-(2-chloro-2′-methyl-3′-(4-oxo-3-(((((R)-5-oxopyrrolidin-2-yl)methyl)amino)methyl)-4H-pyrido[1,2-a]pyrimidin-8-yl)-[1,1′-biphenyl]-3-yl)-2,3-dihydro-1H-inden-2-yl)-3-hydroxypropanamide;
(R)-2-amino-N—((S)-5-(2-chloro-2′-methyl-3′-(4-oxo-3-(((((S)-5-oxopyrrolidin-2-yl)methyl)amino)methyl)-4H-pyrido[1,2-a]pyrimidin-8-yl)-[1,1′-biphenyl]-3-yl)-2,3-dihydro-1H-inden-2-yl)-3-hydroxypropanamide;
(R)-2-acmino-N—((R)-5-(2-chloro-2′-methyl-3′-(4-oxo-3-(((((S)-5-oxopyrrolidin-2-yl)methyl)amino)methyl)-4H-pyrido[1,2-a]pyrimidin-8-yl)-[1,1′-biphenyl]-3-yl)-2,3-dihydro-1H-inden-2-yl)-3-hydroxypropanamide;
(R)-2-amino-N—((S)-5-(2-chloro-2′-methyl-3′-(4-oxo-3-(((((R)-5-oxopyrrolidin-2-yl)methyl)amino)methyl)-4H-pyrido[1,2-a]pyrimidin-8-yl)-[1,1′-biphenyl]-3-yl)-2,3-dihydro-1H-inden-2-yl)-3-hydroxypropanamide;
(R)-2-amino-N—((R)-5-(2-chloro-2′-methyl-3′-(4-oxo-3-(((((R)-5-oxopyrrolidin-2-yl)methyl)amino)methyl)-4H-pyrido[1,2-a]pyrimidin-8-yl)-[1,1′-biphenyl]-3-yl)-2,3-dihydro-1H-inden-2-yl)-3-hydroxypropanamide;
(S)—N—((S)-5-(2-chloro-2′-methyl-3′-(4-oxo-3-(((((S)-5-oxopyrrolidin-2-yl)methyl)amino)methyl)-4H-pyrido[1,2-a]pyrimidin-8-yl)-[1,1′-biphenyl]-3-yl)-2,3-dihydro-1H-inden-2-yl)pyrrolidine-2-carboxamide;
(S)—N—((R)-5-(2-chloro-2′-methyl-3′-(4-oxo-3-(((((S)-5-oxopyrrolidin-2-yl)methyl)amino)methyl)-4H-pyrido[1,2-a]pyrimidin-8-yl)-[1,1′-biphenyl]-3-yl)-2,3-dihydro-1H-inden-2-yl)pyrrolidine-2-carboxamide;
(S)—N—((S)-5-(2-chloro-2′-methyl-3′-(4-oxo-3-(((((R)-5-oxopyrrolidin-2-yl)methyl)amino)methyl)-4H-pyrido[1,2-a]pyrimidin-8-yl)-[1,1′-biphenyl]-3-yl)-2,3-dihydro-1H-inden-2-yl)pyrrolidine-2-carboxamide;
(S)—N—((R)-5-(2-chloro-2′-methyl-3′-(4-oxo-3-(((((R)-5-oxopyrrolidin-2-yl)methyl)amino)methyl)-4H-pyrido[1,2-a]pyrimidin-8-yl)-[1,1′-biphenyl]-3-yl)-2,3-dihydro-1H-inden-2-yl)pyrrolidine-2-carboxamide;
(R)—N—((S)-5-(2-chloro-2′-methyl-3′-(4-oxo-3-(((((S)-5-oxopyrrolidin-2-yl)methyl)amino)methyl)-4H-pyrido[1,2-a]pyrimidin-8-yl)-[1,1′-biphenyl]-3-yl)-2,3-dihydro-1H-inden-2-yl)pyrrolidine-2-carboxamide;
(R)—N—((R)-5-(2-chloro-2′-methyl-3′-(4-oxo-3-(((((S)-5-oxopyrrolidin-2-yl)methyl)amino)methyl)-4H-pyrido[1,2-a]pyrimidin-8-yl)-[1,1′-biphenyl]-3-yl)-2,3-dihydro-1H-inden-2-yl)pyrrolidine-2-carboxamide;
(R)—N—((S)-5-(2-chloro-2′-methyl-3′-(4-oxo-3-(((((R)-5-oxopyrrolidin-2-yl)methyl)amino)methyl)-4H-pyrido[1,2-a]pyrimidin-8-yl)-[1,1′-biphenyl]-3-yl)-2,3-dihydro-1H-inden-2-yl)pyrrolidine-2-carboxamide;
(R)—N—((R)-5-(2-chloro-2′-methyl-3′-(4-oxo-3-(((((R)-5-oxopyrrolidin-2-yl)methyl)amino)methyl)-4H-pyrido[1,2-a]pyrimidin-8-yl)-[1,1′-biphenyl]-3-yl)-2,3-dihydro-1H-inden-2-yl)pyrrolidine-2-carboxamide;
8-(2,2′-dichloro-3′-(6-methoxy-5-(((((S)-5-oxopyrrolidin-2-yl)methyl)amino)methyl)pyrazin-2-yl)-[1,1′-biphenyl]-3-yl)-3-(((((S)-5-oxopyrrolidin-2-yl)methyl)amino)methyl)-4H-pyrido[1,2-a]pyrimidin-4-one;
8-(2,2′-dichloro-3′-(6-methoxy-5-(((((S)-5-oxopyrrolidin-2-yl)methyl)amino)methyl)pyrazin-2-yl)-[1,1′-biphenyl]-3-yl)-3-(((((R)-5-oxopyrrolidin-2-yl)methyl)amino)methyl)-4H-pyrido[1,2-a]pyrimidin-4-one;
8-(2,2′-dichloro-3′-(6-methoxy-5-(((((R)-5-oxopyrrolidin-2-yl)methyl)amino)methyl)pyrazin-2-yl)-[1,1′-biphenyl]-3-yl)-3-(((((S)-5-oxopyrrolidin-2-yl)methyl)amino)methyl)-4H-pyrido[1,2-a]pyrimidin-4-one;
8-(2,2′-dichloro-3′-(6-methoxy-5-(((((R)-5-oxopyrrolidin-2-yl)methyl)amino)methyl)pyrazin-2-yl)-[1,1′-biphenyl]-3-yl)-3-(((((R)-5-oxopyrrolidin-2-yl)methyl)amino)methyl)-4H-pyrido[1,2-a]pyrimidin-4-one;
8-(2,2′-dichloro-3′-(4-oxo-3-(((((S)-5-oxopyrrolidin-2-yl)methyl)amino)methyl)-4H-pyrido[1,2-a]pyrimidin-8-yl)-[1,1′-biphenyl]-3-yl)-3-(((((S)-5-oxopyrrolidin-2-yl)methyl)amino)methyl)-4H-pyrido[1,2-a]pyrimidin-4-one;
8-(2,2′-dichloro-3′-(4-oxo-3-(((((R)-5-oxopyrrolidin-2-yl)methyl)amino)methyl)-4H-pyrido[1,2-a]pyrimidin-8-yl)-[1,1′-biphenyl]-3-yl)-3-(((((S)-5-oxopyrrolidin-2-yl)methyl)amino)methyl)-4H-pyrido[1,2-a]pyrimidin-4-one;
8-(2,2′-dichloro-3′-(4-oxo-3-(((((S)-5-oxopyrrolidin-2-yl)methyl)amino)methyl)-4H-pyrido[1,2-a]pyrimidin-8-yl)-[1,1′-biphenyl]-3-yl)-3-(((((R)-5-oxopyrrolidin-2-yl)methyl)amino)methyl)-4H-pyrido[1,2-a]pyrimidin-4-one;
8-(2,2′-dichloro-3′-(4-oxo-3-(((((R)-5-oxopyrrolidin-2-yl)methyl)amino)methyl)-4H-pyrido[1,2-a]pyrimidin-8-yl)-[1,1′-biphenyl]-3-yl)-3-(((((R)-5-oxopyrrolidin-2-yl)methyl)amino)methyl)-4H-pyrido[1,2-a]pyrimidin-4-one;
8-(2′-chloro-3′-(6-methoxy-5-(((((S)-5-oxopyrrolidin-2-yl)methyl)amino)methyl)pyrazin-2-yl)-2-methyl-[1,1′-biphenyl]-3-yl)-3-(((((S)-5-oxopyrrolidin-2-yl)methyl)amino)methyl)-4H-quinolizin-4-one;
8-(2′-chloro-3′-(6-methoxy-5-(((((R)-5-oxopyrrolidin-2-yl)methyl)amino)methyl)pyrazin-2-yl)-2-methyl-[1,1′-biphenyl]-3-yl)-3-(((((S)-5-oxopyrrolidin-2-yl)methyl)amino)methyl)-4H-quinolizin-4-one;
8-(2′-chloro-3′-(6-methoxy-5-(((((S)-5-oxopyrrolidin-2-yl)methyl)amino)methyl)pyrazin-2-yl)-2-methyl-[1,1′-biphenyl]-3-yl)-3-(((((R)-5-oxopyrrolidin-2-yl)methyl)amino)methyl)-4H-quinolizin-4-one;
8-(2′-chloro-3′-(6-methoxy-5-(((((R)-5-oxopyrrolidin-2-yl)methyl)amino)methyl)pyrazin-2-yl)-2-methyl-[1,1′-biphenyl]-3-yl)-3-(((((R)-5-oxopyrrolidin-2-yl)methyl)amino)methyl)-4H-quinolizin-4-one;
8-(2′-chloro-3′-(6-chloro-5-(((((S)-5-oxopyrrolidin-2-yl)methyl)amino)methyl)pyridin-2-yl)-2-methyl-[1,1′-biphenyl]-3-yl)-3-(((((S)-5-oxopyrrolidin-2-yl)methyl)amino)methyl)-4H-pyrido[1,2-a]pyrimidin-4-one;
8-(2′-chloro-3′-(6-chloro-5-(((((S)-5-oxopyrrolidin-2-yl)methyl)amino)methyl)pyridin-2-yl)-2-methyl-[1,1′-biphenyl]-3-yl)-3-(((((R)-5-oxopyrrolidin-2-yl)methyl)amino)methyl)-4H-pyrido[1,2-a]pyrimidin-4-one;
8-(2′-chloro-3′-(6-chloro-5-(((((R)-5-oxopyrrolidin-2-yl)methyl)amino)methyl)pyridin-2-yl)-2-methyl-[1,1′-biphenyl]-3-yl)-3-(((((S)-5-oxopyrrolidin-2-yl)methyl)amino)methyl)-4H-pyrido[1,2-a]pyrimidin-4-one;
8-(2′-chloro-3′-(6-chloro-5-(((((R)-5-oxopyrrolidin-2-yl)methyl)amino)methyl)pyridin-2-yl)-2-methyl-[1,1′-biphenyl]-3-yl)-3-(((((R)-5-oxopyrrolidin-2-yl)methyl)amino)methyl)-4H-pyrido[1,2-a]pyrimidin-4-one;
(S)-5-((((6-(2,2′-dichloro-3′-((S)-8-methyl-5-((((S)-5-oxopyrrolidin-2-yl)methyl)amino)-5,6,7,8-tetrahydro-1,8-naphthyridin-2-yl)-[1,1′-biphenyl]-3-yl)-2-methoxypyridin-3-yl)methyl)amino)methyl)pyrrolidin-2-one;
(S)-5-((((6-(2,2′-dichloro-3′-((S)-8-methyl-5-((((R)-5-oxopyrrolidin-2-yl)methyl)amino)-5,6,7,8-tetrahydro-1,8-naphthyridin-2-yl)-[1,1′-biphenyl]-3-yl)-2-methoxypyridin-3-yl)methyl)amino)methyl)pyrrolidin-2-one;
(S)-5-((((6-(2,2′-dichloro-3′-((R)-8-methyl-5-((((S)-5-oxopyrrolidin-2-yl)methyl)amino)-5,6,7,8-tetrahydro-1,8-naphthyridin-2-yl)-[1,1′-biphenyl]-3-yl)-2-methoxypyridin-3-yl)methyl)amino)methyl)pyrrolidin-2-one;
(S)-5-((((6-(2,2′-dichloro-3′-((R)-8-methyl-5-((((R)-5-oxopyrrolidin-2-yl)methyl)amino)-5,6,7,8-tetrahydro-1,8-naphthyridin-2-yl)-[1,1′-biphenyl]-3-yl)-2-methoxypyridin-3-yl)methyl)amino)methyl)pyrrolidin-2-one;
(R)-5-((((6-(2,2′-dichloro-3′-((S)-8-methyl-5-((((S)-5-oxopyrrolidin-2-yl)methyl)amino)-5,6,7,8-tetrahydro-1,8-naphthyridin-2-yl)-[1,1′-biphenyl]-3-yl)-2-methoxypyridin-3-yl)methyl)amino)methyl)pyrrolidin-2-one;
(R)-5-((((6-(2,2′-dichloro-3′-((S)-8-methyl-5-((((R)-5-oxopyrrolidin-2-yl)methyl)amino)-5,6,7,8-tetrahydro-1,8-naphthyridin-2-yl)-[1,1′-biphenyl]-3-yl)-2-methoxypyridin-3-yl)methyl)amino)methyl)pyrrolidin-2-one;
(R)-5-((((6-(2,2′-dichloro-3′-((R)-8-methyl-5-((((S)-5-oxopyrrolidin-2-yl)methyl)amino)-5,6,7,8-tetrahydro-1,8-naphthyridin-2-yl)-[1,1′-biphenyl]-3-yl)-2-methoxypyridin-3-yl)methyl)amino)methyl)pyrrolidin-2-one;
(R)-5-((((6-(2,2′-dichloro-3′-((R)-8-methyl-5-((((R)-5-oxopyrrolidin-2-yl)methyl)amino)-5,6,7,8-tetrahydro-1,8-naphthyridin-2-yl)-[1,1′-biphenyl]-3-yl)-2-methoxypyridin-3-yl)methyl)amino)methyl)pyrrolidin-2-one;
8-(2′-chloro-3′-(6-methoxy-5-(((((S)-5-oxopyrrolidin-2-yl)methyl)amino)methyl)pyridin-2-yl)-2-methyl-[1,1′-biphenyl]-3-yl)-3-((methyl(((S)-5-oxopyrrolidin-2-yl)methyl)amino)methyl)-4H-pyrido[1,2-a]pyrimidin-4-one;
8-(2′-chloro-3′-(6-methoxy-5-(((((R)-5-oxopyrrolidin-2-yl)methyl)amino)methyl)pyridin-2-yl)-2-methyl-[1,1′-biphenyl]-3-yl)-3-((methyl(((S)-5-oxopyrrolidin-2-yl)methyl)amino)methyl)-4H-pyrido[1,2-a]pyrimidin-4-one;
8-(2′-chloro-3′-(6-methoxy-5-(((((S)-5-oxopyrrolidin-2-yl)methyl)amino)methyl)pyridin-2-yl)-2-methyl-[1,1′-biphenyl]-3-yl)-3-((methyl(((R)-5-oxopyrrolidin-2-yl)methyl)amino)methyl)-4H-pyrido[1,2-a]pyrimidin-4-one;
8-(2′-chloro-3′-(6-methoxy-5-(((((R)-5-oxopyrrolidin-2-yl)methyl)amino)methyl)pyridin-2-yl)-2-methyl-[1,1′-biphenyl]-3-yl)-3-((methyl(((R)-5-oxopyrrolidin-2-yl)methyl)amino)methyl)-4H-pyrido[1,2-a]pyrimidin-4-one;
(S)-8-(2′-chloro-3′-(5-((3-hydroxyazetidin-1-yl)methyl)-6-methoxypyridin-2-yl)-2-methyl-[1,1′-biphenyl]-3-yl)-3-((methyl((5-oxopyrrolidin-2-yl)methyl)amino)methyl)-4H-pyrido[1,2-a]pyrimidin-4-one;
(R)-8-(2′-chloro-3′-(5-((3-hydroxyazetidin-1-yl)methyl)-6-methoxypyridin-2-yl)-2-methyl-[1,1′-biphenyl]-3-yl)-3-((methyl((5-oxopyrrolidin-2-yl)methyl)amino)methyl)-4H-pyrido[1,2-a]pyrimidin-4-one;
(S)-5-((((5-(2,2′-dichloro-3′-((S)-8-methyl-5-((((S)-5-oxopyrrolidin-2-yl)methyl)amino)-5,6,7,8-tetrahydro-1,8-naphthyridin-2-yl)-[1,1′-biphenyl]-3-yl)-3-methoxypyrazin-2-yl)methyl)amino)methyl)pyrrolidin-2-one;
(S)-5-((((5-(2,2′-dichloro-3′-((R)-8-methyl-5-((((S)-5-oxopyrrolidin-2-yl)methyl)amino)-5,6,7,8-tetrahydro-1,8-naphthyridin-2-yl)-[1,1′-biphenyl]-3-yl)-3-methoxypyrazin-2-yl)methyl)amino)methyl)pyrrolidin-2-one;
(S)-5-((((5-(2,2′-dichloro-3′-((S)-8-methyl-5-((((R)-5-oxopyrrolidin-2-yl)methyl)amino)-5,6,7,8-tetrahydro-1,8-naphthyridin-2-yl)-[1,1′-biphenyl]-3-yl)-3-methoxypyrazin-2-yl)methyl)amino)methyl)pyrrolidin-2-one;
(S)-5-((((5-(2,2′-dichloro-3′-((R)-8-methyl-5-((((R)-5-oxopyrrolidin-2-yl)methyl)amino)-5,6,7,8-tetrahydro-1,8-naphthyridin-2-yl)-[1,1′-biphenyl]-3-yl)-3-methoxypyrazin-2-yl)methyl)amino)methyl)pyrrolidin-2-one;
(R)-5-((((5-(2,2′-dichloro-3′-((S)-8-methyl-5-((((S)-5-oxopyrrolidin-2-yl)methyl)amino)-5,6,7,8-tetrahydro-1,8-naphthyridin-2-yl)-[1,1′-biphenyl]-3-yl)-3-methoxypyrazin-2-yl)methyl)amino)methyl)pyrrolidin-2-one;
(R)-5-((((5-(2,2′-dichloro-3′-((R)-8-methyl-5-((((S)-5-oxopyrrolidin-2-yl)methyl)amino)-5,6,7,8-tetrahydro-1,8-naphthyridin-2-yl)-[1,1′-biphenyl]-3-yl)-3-methoxypyrazin-2-yl)methyl)amino)methyl)pyrrolidin-2-one;
(R)-5-((((5-(2,2′-dichloro-3′-((S)-8-methyl-5-((((R)-5-oxopyrrolidin-2-yl)methyl)amino)-5,6,7,8-tetrahydro-1,8-naphthyridin-2-yl)-[1,1′-biphenyl]-3-yl)-3-methoxypyrazin-2-yl)methyl)amino)methyl)pyrrolidin-2-one;
(R)-5-((((5-(2,2′-dichloro-3′-((R)-8-methyl-5-((((R)-5-oxopyrrolidin-2-yl)methyl)amino)-5,6,7,8-tetrahydro-1,8-naphthyridin-2-yl)-[1,1′-biphenyl]-3-yl)-3-methoxypyrazin-2-yl)methyl)amino)methyl)pyrrolidin-2-one;
8-(2′-chloro-3′-(6-fluoro-5-(((((S)-5-oxopyrrolidin-2-yl)methyl)amino)methyl)pyridin-2-yl)-2-methyl-[1,1′-biphenyl]-3-yl)-3-(((((S)-5-oxopyrrolidin-2-yl)methyl)amino)methyl)-4H-pyrido[1,2-a]pyrimidin-4-one;
8-(2′-chloro-3′-(6-fluoro-5-(((((R)-5-oxopyrrolidin-2-yl)methyl)amino)methyl)pyridin-2-yl)-2-methyl-[1,1′-biphenyl]-3-yl)-3-(((((S)-5-oxopyrrolidin-2-yl)methyl)amino)methyl)-4H-pyrido[1,2-a]pyrimidin-4-one;
8-(2′-chloro-3′-(6-fluoro-5-(((((S)-5-oxopyrrolidin-2-yl)methyl)amino)methyl)pyridin-2-yl)-2-methyl-[1,1′-biphenyl]-3-yl)-3-(((((R)-5-oxopyrrolidin-2-yl)methyl)amino)methyl)-4H-pyrido[1,2-a]pyrimidin-4-one;
8-(2′-chloro-3′-(6-fluoro-5-(((((R)-5-oxopyrrolidin-2-yl)methyl)amino)methyl)pyridin-2-yl)-2-methyl-[1,1′-biphenyl]-3-yl)-3-(((((R)-5-oxopyrrolidin-2-yl)methyl)amino)methyl)-4H-pyrido[1,2-a]pyrimidin-4-one;
2-(2,2′-dichloro-3′-(6-methoxy-5-(((((S)-5-oxopyrrolidin-2-yl)methyl)amino)methyl)pyrazin-2-yl)-[1,1′-biphenyl]-3-yl)-7-(((((S)-5-oxopyrrolidin-2-yl)methyl)amino)methyl)-4H-pyrido[1,2-a]pyrimidin-4-one;
2-(2,2′-dichloro-3′-(6-methoxy-5-(((((R)-5-oxopyrrolidin-2-yl)methyl)amino)methyl)pyrazin-2-yl)-[1,1′-biphenyl]-3-yl)-7-(((((S)-5-oxopyrrolidin-2-yl)methyl)amino)methyl)-4H-pyrido[1,2-a]pyrimidin-4-one;
2-(2,2′-dichloro-3′-(6-methoxy-5-(((((S)-5-oxopyrrolidin-2-yl)methyl)amino)methyl)pyrazin-2-yl)-[1,1′-biphenyl]-3-yl)-7-(((((R)-5-oxopyrrolidin-2-yl)methyl)amino)methyl)-4H-pyrido[1,2-a]pyrimidin-4-one;
2-(2,2′-dichloro-3′-(6-methoxy-5-(((((R)-5-oxopyrrolidin-2-yl)methyl)amino)methyl)pyrazin-2-yl)-[1,1′-biphenyl]-3-yl)-7-(((((R)-5-oxopyrrolidin-2-yl)methyl)amino)methyl)-4H-pyrido[1,2-a]pyrimidin-4-one;
2-(2,2′-dichloro-3′-(6-methoxy-5-(((((S)-5-oxopyrrolidin-2-yl)methyl)amino)methyl)pyridin-2-yl)-[1,1′-biphenyl]-3-yl)-7-(((((S)-5-oxopyrrolidin-2-yl)methyl)amino)methyl)-4H-pyrido[1,2-a]pyrimidin-4-one;
2-(2,2′-dichloro-3′-(6-methoxy-5-(((((R)-5-oxopyrrolidin-2-yl)methyl)amino)methyl)pyridin-2-yl)-[1,1′-biphenyl]-3-yl)-7-(((((S)-5-oxopyrrolidin-2-yl)methyl)amino)methyl)-4H-pyrido[1,2-a]pyrimidin-4-one;
2-(2,2′-dichloro-3′-(6-methoxy-5-(((((S)-5-oxopyrrolidin-2-yl)methyl)amino)methyl)pyridin-2-yl)-[1,1′-biphenyl]-3-yl)-7-(((((R)-5-oxopyrrolidin-2-yl)methyl)amino)methyl)-4H-pyrido[1,2-2-(2,2′-dichloro-3′-(6-methoxy-5-(((((R)-5-oxopyrrolidin-2-yl)methyl)amino)methyl)pyridin-2-yl)-[1,1′-biphenyl]-3-yl)-7-(((((R)-5-oxopyrrolidin-2-yl)methyl)amino)methyl)-4H-pyrido[1,2-a]pyrimidin-4-one;
2-(2,2′-dichloro-3′-(4-oxo-7-(((((S)-5-oxopyrrolidin-2-yl)methyl)amino)methyl)-4H-pyrido[1,2-a]pyrimidin-2-yl)-[1,1′-biphenyl]-3-yl)-7-(((((S)-5-oxopyrrolidin-2-yl)methyl)amino)methyl)-4H-pyrido[1,2-a]pyrimidin-4-one;
2-(2,2′-dichloro-3′-(4-oxo-7-(((((S)-5-oxopyrrolidin-2-yl)methyl)amino)methyl)-4H-pyrido[1,2-a]pyrimidin-2-yl)-[1,1′-biphenyl]-3-yl)-7-(((((R)-5-oxopyrrolidin-2-yl)methyl)amino)methyl)-4H-pyrido[1,2-a]pyrimidin-4-one;
2-(2,2′-dichloro-3′-(4-oxo-7-(((((R)-5-oxopyrrolidin-2-yl)methyl)amino)methyl)-4H-pyrido[1,2-a]pyrimidin-2-yl)-[1,1′-biphenyl]-3-yl)-7-(((((S)-5-oxopyrrolidin-2-yl)methyl)amino)methyl)-4H-pyrido[1,2-a]pyrimidin-4-one;
2-(2,2′-dichloro-3′-(4-oxo-7-(((((R)-5-oxopyrrolidin-2-yl)methyl)amino)methyl)-4H-pyrido[1,2-a]pyrimidin-2-yl)-[1,1′-biphenyl]-3-yl)-7-(((((R)-5-oxopyrrolidin-2-yl)methyl)amino)methyl)-4H-pyrido[1,2-a]pyrimidin-4-one;
8-(2′-chloro-2-methyl-3′-(5-(((((S)-5-oxopyrrolidin-2-yl)methyl)amino)methyl)pyridin-2-yl)-[1,1′-biphenyl]-3-yl)-3-(((((S)-5-oxopyrrolidin-2-yl)methyl)amino)methyl)-4H-pyrido[1,2-a]pyrimidin-4-one;
8-(2′-chloro-2-methyl-3′-(5-(((((R)-5-oxopyrrolidin-2-yl)methyl)amino)methyl)pyridin-2-yl)-[1,1′-biphenyl]-3-yl)-3-(((((S)-5-oxopyrrolidin-2-yl)methyl)amino)methyl)-4H-pyrido[1,2-a]pyrimidin-4-one;
8-(2′-chloro-2-methyl-3′-(5-(((((S)-5-oxopyrrolidin-2-yl)methyl)amino)methyl)pyridin-2-yl)-[1,1′-biphenyl]-3-yl)-3-(((((R)-5-oxopyrrolidin-2-yl)methyl)amino)methyl)-4H-pyrido[1,2-a]pyrimidin-4-one;
8-(2′-chloro-2-methyl-3′-(5-(((((R)-5-oxopyrrolidin-2-yl)methyl)amino)methyl)pyridin-2-yl)-[1,1′-biphenyl]-3-yl)-3-(((((R)-5-oxopyrrolidin-2-yl)methyl)amino)methyl)-4H-pyrido[1,2-a]pyrimidin-4-one;
8-(2′-chloro-3′-(6-fluoro-5-(((((S)-5-oxopyrrolidin-2-yl)methyl)amino)methyl)pyridin-2-yl)-2-methyl-[1,1′-biphenyl]-3-yl)-3-((((S)-5-oxopyrrolidin-3-yl)amino)methyl)-4H-pyrido[1,2-a]pyrimidin-4-one;
8-(2′-chloro-3′-(6-fluoro-5-(((((R)-5-oxopyrrolidin-2-yl)methyl)amino)methyl)pyridin-2-yl)-2-methyl-[1,1′-biphenyl]-3-yl)-3-((((S)-5-oxopyrrolidin-3-yl)amino)methyl)-4H-pyrido[1,2-a]pyrimidin-4-one;
8-(2′-chloro-3′-(6-fluoro-5-(((((S)-5-oxopyrrolidin-2-yl)methyl)amino)methyl)pyridin-2-yl)-2-methyl-[1,1′-biphenyl]-3-yl)-3-((((R)-5-oxopyrrolidin-3-yl)amino)methyl)-4H-pyrido[1,2-a]pyrimidin-4-one;
8-(2′-chloro-3′-(6-fluoro-5-(((((R)-5-oxopyrrolidin-2-yl)methyl)amino)methyl)pyridin-2-yl)-2-methyl-[1,1′-biphenyl]-3-yl)-3-((((R)-5-oxopyrrolidin-3-yl)amino)methyl)-4H-pyrido[1,2-a]pyrimidin-4-one;
8-(2,2′-dichloro-3′-(6-methoxy-5-((((R)-5-oxopyrrolidin-3-yl)amino)methyl)pyridin-2-yl)-[1,1′-biphenyl]-3-yl)-3-((((R)-5-oxopyrrolidin-3-yl)amino)methyl)-4H-pyrido[1,2-a]pyrimidin-4-one;
8-(2,2′-dichloro-3′-(6-methoxy-5-((((R)-5-oxopyrrolidin-3-yl)amino)methyl)pyridin-2-yl)-[1,1′-biphenyl]-3-yl)-3-((((S)-5-oxopyrrolidin-3-yl)amino)methyl)-4H-pyrido[1,2-a]pyrimidin-4-one;
8-(2,2′-dichloro-3′-(6-methoxy-5-((((S)-5-oxopyrrolidin-3-yl)amino)methyl)pyridin-2-yl)-[1,1′-biphenyl]-3-yl)-3-((((R)-5-oxopyrrolidin-3-yl)amino)methyl)-4H-pyrido[1,2-a]pyrimidin-4-one;
8-(2,2′-dichloro-3′-(6-methoxy-5-((((S)-5-oxopyrrolidin-3-yl)amino)methyl)pyridin-2-yl)-[1,1′-biphenyl]-3-yl)-3-((((S)-5-oxopyrrolidin-3-yl)amino)methyl)-4H-pyrido[1,2-a]pyrimidin-4-one;
8-(2′-chloro-3′-(6-(dimethylamino)-5-(((((S)-5-oxopyrrolidin-2-yl)methyl)amino)methyl)pyridin-2-yl)-2-methyl-[1,1′-biphenyl]-3-yl)-3-(((((S)-5-oxopyrrolidin-2-yl)methyl)amino)methyl)-4H-pyrido[1,2-a]pyrimidin-4-one;
8-(2′-chloro-3′-(6-(dimethylamino)-5-(((((R)-5-oxopyrrolidin-2-yl)methyl)amino)methyl)pyridin-2-yl)-2-methyl-[1,1′-biphenyl]-3-yl)-3-(((((S)-5-oxopyrrolidin-2-yl)methyl)amino)methyl)-4H-pyrido[1,2-a]pyrimidin-4-one;
8-(2′-chloro-3′-(6-(dimethylamino)-5-(((((S)-5-oxopyrrolidin-2-yl)methyl)amino)methyl)pyridin-2-yl)-2-methyl-[1,1′-biphenyl]-3-yl)-3-(((((R)-5-oxopyrrolidin-2-yl)methyl)amino)methyl)-4H-pyrido[1,2-a]pyrimidin-4-one;
8-(2′-chloro-3′-(6-(dimethylamino)-5-(((((R)-5-oxopyrrolidin-2-yl)methyl)amino)methyl)pyridin-2-yl)-2-methyl-[1,1′-biphenyl]-3-yl)-3-(((((R)-5-oxopyrrolidin-2-yl)methyl)amino)methyl)-4H-pyrido[1,2-a]pyrimidin-4-one;
8-(2′-chloro-2-methyl-4″-(((((S)-5-oxopyrrolidin-2-yl)methyl)amino)methyl)-[1,1′:3′,1″-terphenyl]-3-yl)-3-(((((S)-5-oxopyrrolidin-2-yl)methyl)amino)methyl)-4H-pyrido[1,2-a]pyrimidin-4-one;
8-(2′-chloro-2-methyl-4″-(((((R)-5-oxopyrrolidin-2-yl)methyl)amino)methyl)-[1,1′:3′,1″-terphenyl]-3-yl)-3-(((((S)-5-oxopyrrolidin-2-yl)methyl)amino)methyl)-4H-pyrido[1,2-a]pyrimidin-4-one;
8-(2′-chloro-2-methyl-4″-(((((S)-5-oxopyrrolidin-2-yl)methyl)amino)methyl)-[1,1′:3′,1″-terphenyl]-3-yl)-3-(((((R)-5-oxopyrrolidin-2-yl)methyl)amino)methyl)-4H-pyrido[1,2-a]pyrimidin-4-one;
8-(2′-chloro-2-methyl-4″-(((((R)-5-oxopyrrolidin-2-yl)methyl)amino)methyl)-[1,1′:3′,1″-terphenyl]-3-yl)-3-(((((R)-5-oxopyrrolidin-2-yl)methyl)amino)methyl)-4H-pyrido[1,2-a]pyrimidin-4-one;
8-(2′-chloro-3″-methoxy-2-methyl-4″-(((((S)-5-oxopyrrolidin-2-yl)methyl)amino)methyl)-[1,1′:3′,1″-terphenyl]-3-yl)-3-(((((S)-5-oxopyrrolidin-2-yl)methyl)amino)methyl)-4H-pyrido[1,2-a]pyrimidin-4-one;
8-(2′-chloro-3″-methoxy-2-methyl-4″-(((((R)-5-oxopyrrolidin-2-yl)methyl)amino)methyl)-[1,1′:3′,1″-terphenyl]-3-yl)-3-(((((S)-5-oxopyrrolidin-2-yl)methyl)amino)methyl)-4H-pyrido[1,2-a]pyrimidin-4-one;
8-(2′-chloro-3″-methoxy-2-methyl-4″-(((((S)-5-oxopyrrolidin-2-yl)methyl)amino)methyl)-[1,1′:3′,1″-terphenyl]-3-yl)-3-(((((R)-5-oxopyrrolidin-2-yl)methyl)amino)methyl)-4H-pyrido[1,2-a]pyrimidin-4-one;
8-(2′-chloro-3″-methoxy-2-methyl-4″-(((((R)-5-oxopyrrolidin-2-yl)methyl)amino)methyl)-[1,1′:3′,1″-terphenyl]-3-yl)-3-(((((R)-5-oxopyrrolidin-2-yl)methyl)amino)methyl)-4H-pyrido[1,2-a]pyrimidin-4-one;
8-(2′,3″-dichloro-2-methyl-4″-(((((S)-5-oxopyrrolidin-2-yl)methyl)amino)methyl)-[1,1′:3′,1″-terphenyl]-3-yl)-3-(((((S)-5-oxopyrrolidin-2-yl)methyl)amino)methyl)-4H-pyrido[1,2-a]pyrimidin-4-one;
8-(2′,3″-dichloro-2-methyl-4″-(((((R)-5-oxopyrrolidin-2-yl)methyl)amino)methyl)-[1,1′:3′,1″-terphenyl]-3-yl)-3-(((((S)-5-oxopyrrolidin-2-yl)methyl)amino)methyl)-4H-pyrido[1,2-a]pyrimidin-4-one;
8-(2′,3″-dichloro-2-methyl-4″-(((((S)-5-oxopyrrolidin-2-yl)methyl)amino)methyl)-[1,1′:3′,1″-terphenyl]-3-yl)-3-(((((R)-5-oxopyrrolidin-2-yl)methyl)amino)methyl)-4H-pyrido[1,2-a]pyrimidin-4-one;
8-(2′,3″-dichloro-2-methyl-4″-(((((R)-5-oxopyrrolidin-2-yl)methyl)amino)methyl)-[1,1′:3′,1″-terphenyl]-3-yl)-3-(((((R)-5-oxopyrrolidin-2-yl)methyl)amino)methyl)-4H-pyrido[1,2-a]pyrimidin-4-one;
8-(2′-chloro-2-methyl-3′-((R)-8-methyl-5-((((R)-5-oxopyrrolidin-2-yl)methyl)amino)-5,6,7,8-tetrahydro-1,8-naphthyridin-2-yl)-[1,1′-biphenyl]-3-yl)-3-(((((R)-5-oxopyrrolidin-2-yl)methyl)amino)methyl)-4H-pyrido[1,2-a]pyrimidin-4-one;
8-(2′-chloro-2-methyl-3′-((R)-8-methyl-5-((((R)-5-oxopyrrolidin-2-yl)methyl)amino)-5,6,7,8-tetrahydro-1,8-naphthyridin-2-yl)-[1,1′-biphenyl]-3-yl)-3-(((((S)-5-oxopyrrolidin-2-yl)methyl)amino)methyl)-4H-pyrido[1,2-a]pyrimidin-4-one;
8-(2′-chloro-2-methyl-3′-((R)-8-methyl-5-((((S)-5-oxopyrrolidin-2-yl)methyl)amino)-5,6,7,8-tetrahydro-1,8-naphthyridin-2-yl)-[1,1′-biphenyl]-3-yl)-3-(((((R)-5-oxopyrrolidin-2-yl)methyl)amino)methyl)-4H-pyrido[1,2-a]pyrimidin-4-one;
8-(2′-chloro-2-methyl-3′-((R)-8-methyl-5-((((S)-5-oxopyrrolidin-2-yl)methyl)amino)-5,6,7,8-tetrahydro-1,8-naphthyridin-2-yl)-[1,1′-biphenyl]-3-yl)-3-(((((S)-5-oxopyrrolidin-2-yl)methyl)amino)methyl)-4H-pyrido[1,2-a]pyrimidin-4-one;
8-(2′-chloro-2-methyl-3′-((S)-8-methyl-5-((((R)-5-oxopyrrolidin-2-yl)methyl)amino)-5,6,7,8-tetrahydro-1,8-naphthyridin-2-yl)-[1,1′-biphenyl]-3-yl)-3-(((((R)-5-oxopyrrolidin-2-yl)methyl)amino)methyl)-4H-pyrido[1,2-a]pyrimidin-4-one;
8-(2′-chloro-2-methyl-3′-((S)-8-methyl-5-((((R)-5-oxopyrrolidin-2-yl)methyl)amino)-5,6,7,8-tetrahydro-1,8-naphthyridin-2-yl)-[1,1′-biphenyl]-3-yl)-3-(((((S)-5-oxopyrrolidin-2-yl)methyl)amino)methyl)-4H-pyrido[1,2-a]pyrimidin-4-one;
8-(2′-chloro-2-methyl-3′-((S)-8-methyl-5-((((S)-5-oxopyrrolidin-2-yl)methyl)amino)-5,6,7,8-tetrahydro-1,8-naphthyridin-2-yl)-[1,1′-biphenyl]-3-yl)-3-(((((R)-5-oxopyrrolidin-2-yl)methyl)amino)methyl)-4H-pyrido[1,2-a]pyrimidin-4-one;
8-(2′-chloro-2-methyl-3′-((S)-8-methyl-5-((((S)-5-oxopyrrolidin-2-yl)methyl)amino)-5,6,7,8-tetrahydro-1,8-naphthyridin-2-yl)-[1,1′-biphenyl]-3-yl)-3-(((((S)-5-oxopyrrolidin-2-yl)methyl)amino)methyl)-4H-pyrido[1,2-a]pyrimidin-4-one;
N-(2-(((6-(3′-(3-(((2-acetamidoethyl)amino)methyl)-4-oxo-4H-pyrido[1,2-a]pyrimidin-8-yl)-2,2′-dichloro-[1,1′-biphenyl]-3-yl)-2-methoxypyridin-3-yl)methyl)amino)ethyl)acetamide;
8-(2,2′-dichloro-3′-(6-methoxy-5-((7-oxo-2,6-diazaspiro[3.4]octan-2-yl)methyl)pyridin-2-yl)-[1,1′-biphenyl]-3-yl)-3-((7-oxo-2,6-diazaspiro[3.4]octan-2-yl)methyl)-4H-pyrido[1,2-a]pyrimidin-4-one;
(S)-8-(3′-(5-(3-aminoazetidin-1-yl)-6-methoxypyrazin-2-yl)-2,2′-dichloro-[1,1′-biphenyl]-3-yl)-3-((((5-oxopyrrolidin-2-yl)methyl)amino)methyl)-4H-pyrido[1,2-a]pyrimidin-4-one;
(R)-8-(3′-(5-(3-aminoazetidin-1-yl)-6-methoxypyrazin-2-yl)-2,2′-dichloro-[1,1′-biphenyl]-3-yl)-3-((((5-oxopyrrolidin-2-yl)methyl)amino)methyl)-4H-pyrido[1,2-a]pyrimidin-4-one;
8-(2′-chloro-3′-(6-ethoxy-5-(((((S)-5-oxopyrrolidin-2-yl)methyl)amino)methyl)pyridin-2-yl)-2-methyl-[1,1′-biphenyl]-3-yl)-3-(((((S)-5-oxopyrrolidin-2-yl)methyl)amino)methyl)-4H-pyrido[1,2-a]pyrimidin-4-one;
8-(2′-chloro-3′-(6-ethoxy-5-(((((R)-5-oxopyrrolidin-2-yl)methyl)amino)methyl)pyridin-2-yl)-2-methyl-[1,1′-biphenyl]-3-yl)-3-(((((S)-5-oxopyrrolidin-2-yl)methyl)amino)methyl)-4H-pyrido[1,2-a]pyrimidin-4-one;
8-(2′-chloro-3′-(6-ethoxy-5-(((((S)-5-oxopyrrolidin-2-yl)methyl)amino)methyl)pyridin-2-yl)-2-methyl-[1,1′-biphenyl]-3-yl)-3-(((((R)-5-oxopyrrolidin-2-yl)methyl)amino)methyl)-4H-pyrido[1,2-a]pyrimidin-4-one;
8-(2′-chloro-3′-(6-ethoxy-5-(((((R)-5-oxopyrrolidin-2-yl)methyl)amino)methyl)pyridin-2-yl)-2-methyl-[1,1′-biphenyl]-3-yl)-3-(((((R)-5-oxopyrrolidin-2-yl)methyl)amino)methyl)-4H-pyrido[1,2-a]pyrimidin-4-one;
8-(2′-chloro-2-methyl-3′-(6-(methylamino)-5-(((((S)-5-oxopyrrolidin-2-yl)methyl)amino)methyl)pyridin-2-yl)-[1,1′-biphenyl]-3-yl)-3-(((((S)-5-oxopyrrolidin-2-yl)methyl)amino)methyl)-4H-pyrido[1,2-a]pyrimidin-4-one;
8-(2′-chloro-2-methyl-3′-(6-(methylamino)-5-(((((R)-5-oxopyrrolidin-2-yl)methyl)amino)methyl)pyridin-2-yl)-[1,1′-biphenyl]-3-yl)-3-(((((S)-5-oxopyrrolidin-2-yl)methyl)amino)methyl)-4H-pyrido[1,2-a]pyrimidin-4-one;
8-(2′-chloro-2-methyl-3′-(6-(methylamino)-5-(((((S)-5-oxopyrrolidin-2-yl)methyl)amino)methyl)pyridin-2-yl)-[1,1′-biphenyl]-3-yl)-3-(((((R)-5-oxopyrrolidin-2-yl)methyl)amino)methyl)-4H-pyrido[1,2-a]pyrimidin-4-one;
8-(2′-chloro-2-methyl-3′-(6-(methylamino)-5-(((((R)-5-oxopyrrolidin-2-yl)methyl)amino)methyl)pyridin-2-yl)-[1,1′-biphenyl]-3-yl)-3-(((((R)-5-oxopyrrolidin-2-yl)methyl)amino)methyl)-4H-pyrido[1,2-a]pyrimidin-4-one;
8-(2′-chloro-3′-(6-(ethylamino)-5-(((((S)-5-oxopyrrolidin-2-yl)methyl)amino)methyl)pyridin-2-yl)-2-methyl-[1,1′-biphenyl]-3-yl)-3-(((((S)-5-oxopyrrolidin-2-yl)methyl)amino)methyl)-4H-pyrido[1,2-a]pyrimidin-4-one;
8-(2′-chloro-3′-(6-(ethylamino)-5-(((((R)-5-oxopyrrolidin-2-yl)methyl)amino)methyl)pyridin-2-yl)-2-methyl-[1,1′-biphenyl]-3-yl)-3-(((((S)-5-oxopyrrolidin-2-yl)methyl)amino)methyl)-4H-pyrido[1,2-a]pyrimidin-4-one;
8-(2′-chloro-3′-(6-(ethylamino)-5-(((((S)-5-oxopyrrolidin-2-yl)methyl)amino)methyl)pyridin-2-yl)-2-methyl-[1,1′-biphenyl]-3-yl)-3-(((((R)-5-oxopyrrolidin-2-yl)methyl)amino)methyl)-4H-pyrido[1,2-a]pyrimidin-4-one;
8-(2′-chloro-3′-(6-(ethylamino)-5-(((((R)-5-oxopyrrolidin-2-yl)methyl)amino)methyl)pyridin-2-yl)-2-methyl-[1,1′-biphenyl]-3-yl)-3-(((((R)-5-oxopyrrolidin-2-yl)methyl)amino)methyl)-4H-pyrido[1,2-a]pyrimidin-4-one;
8-(2,2′-dichloro-3′-(5-(((2-hydroxyethyl)amino)methyl)-6-methoxypyridin-2-yl)-[1,1′-biphenyl]-3-yl)-3-(((2-hydroxyethyl)amino)methyl)-4H-pyrido[1,2-a]pyrimidin-4-one;
8-(2′-chloro-2-methyl-3′-((R)-1-((((S)-5-oxopyrrolidin-2-yl)methyl)amino)-2,3-dihydro-1H-inden-5-yl)-[1,1′-biphenyl]-3-yl)-3-(((((S)-5-oxopyrrolidin-2-yl)methyl)amino)methyl)-4H-pyrido[1,2-a]pyrimidin-4-one;
8-(2′-chloro-2-methyl-3′-((R)-1-((((R)-5-oxopyrrolidin-2-yl)methyl)amino)-2,3-dihydro-1H-inden-5-yl)-[1,1′-biphenyl]-3-yl)-3-(((((S)-5-oxopyrrolidin-2-yl)methyl)amino)methyl)-4H-pyrido[1,2-a]pyrimidin-4-one;
8-(2′-chloro-2-methyl-3′-((R)-1-((((S)-5-oxopyrrolidin-2-yl)methyl)amino)-2,3-dihydro-1H-inden-5-yl)-[1,1′-biphenyl]-3-yl)-3-(((((R)-5-oxopyrrolidin-2-yl)methyl)amino)methyl)-4H-pyrido[1,2-a]pyrimidin-4-one;
8-(2′-chloro-2-methyl-3′-((R)-1-((((R)-5-oxopyrrolidin-2-yl)methyl)amino)-2,3-dihydro-1H-inden-5-yl)-[1,1′-biphenyl]-3-yl)-3-(((((R)-5-oxopyrrolidin-2-yl)methyl)amino)methyl)-4H-pyrido[1,2-a]pyrimidin-4-one;
8-(2′-chloro-2-methyl-3′-((S)-1-((((S)-5-oxopyrrolidin-2-yl)methyl)amino)-2,3-dihydro-1H-inden-5-yl)-[1,1′-biphenyl]-3-yl)-3-(((((S)-5-oxopyrrolidin-2-yl)methyl)amino)methyl)-4H-pyrido[1,2-a]pyrimidin-4-one;
8-(2′-chloro-2-methyl-3′-((S)-1-((((R)-5-oxopyrrolidin-2-yl)methyl)amino)-2,3-dihydro-1H-inden-5-yl)-[1,1′-biphenyl]-3-yl)-3-(((((S)-5-oxopyrrolidin-2-yl)methyl)amino)methyl)-4H-pyrido[1,2-a]pyrimidin-4-one;
8-(2′-chloro-2-methyl-3′-((S)-1-((((S)-5-oxopyrrolidin-2-yl)methyl)amino)-2,3-dihydro-1H-inden-5-yl)-[1,1′-biphenyl]-3-yl)-3-(((((R)-5-oxopyrrolidin-2-yl)methyl)amino)methyl)-4H-pyrido[1,2-a]pyrimidin-4-one;
8-(2′-chloro-2-methyl-3′-((S)-1-((((R)-5-oxopyrrolidin-2-yl)methyl)amino)-2,3-dihydro-1H-inden-5-yl)-[1,1′-biphenyl]-3-yl)-3-(((((R)-5-oxopyrrolidin-2-yl)methyl)amino)methyl)-4H-pyrido[1,2-a]pyrimidin-4-one;
8-(2′-chloro-3′-(4-methoxy-5-(((((S)-5-oxopyrrolidin-2-yl)methyl)amino)methyl)pyridin-2-yl)-2-methyl-[1,1′-biphenyl]-3-yl)-3-(((((S)-5-oxopyrrolidin-2-yl)methyl)amino)methyl)-4H-pyrido[1,2-a]pyrimidin-4-one;
8-(2′-chloro-3′-(4-methoxy-5-(((((R)-5-oxopyrrolidin-2-yl)methyl)amino)methyl)pyridin-2-yl)-2-methyl-[1,1′-biphenyl]-3-yl)-3-(((((S)-5-oxopyrrolidin-2-yl)methyl)amino)methyl)-4H-pyrido[1,2-a]pyrimidin-4-one;
8-(2′-chloro-3′-(4-methoxy-5-(((((S)-5-oxopyrrolidin-2-yl)methyl)amino)methyl)pyridin-2-yl)-2-methyl-[1,1′-biphenyl]-3-yl)-3-(((((R)-5-oxopyrrolidin-2-yl)methyl)amino)methyl)-4H-pyrido[1,2-a]pyrimidin-4-one;
8-(2′-chloro-3′-(4-methoxy-5-(((((R)-5-oxopyrrolidin-2-yl)methyl)amino)methyl)pyridin-2-yl)-2-methyl-[1,1′-biphenyl]-3-yl)-3-(((((R)-5-oxopyrrolidin-2-yl)methyl)amino)methyl)-4H-pyrido[1,2-a]pyrimidin-4-one;
8-(2′-chloro-2-methyl-3′-(5-(((((S)-5-oxopyrrolidin-2-yl)methyl)amino)methyl)-6-(trifluoromethyl)pyridin-2-yl)-[1,1′-biphenyl]-3-yl)-3-(((((S)-5-oxopyrrolidin-2-yl)methyl)amino)methyl)-4H-pyrido[1,2-a]pyrimidin-4-one;
8-(2′-chloro-2-methyl-3′-(5-(((((R)-5-oxopyrrolidin-2-yl)methyl)amino)methyl)-6-(trifluoromethyl)pyridin-2-yl)-[1,1′-biphenyl]-3-yl)-3-(((((S)-5-oxopyrrolidin-2-yl)methyl)amino)methyl)-4H-pyrido[1,2-a]pyrimidin-4-one;
8-(2′-chloro-2-methyl-3′-(5-(((((S)-5-oxopyrrolidin-2-yl)methyl)amino)methyl)-6-(trifluoromethyl)pyridin-2-yl)-[1,1′-biphenyl]-3-yl)-3-(((((R)-5-oxopyrrolidin-2-yl)methyl)amino)methyl)-4H-pyrido[1,2-a]pyrimidin-4-one;
8-(2′-chloro-2-methyl-3′-(5-(((((R)-5-oxopyrrolidin-2-yl)methyl)amino)methyl)-6-(trifluoromethyl)pyridin-2-yl)-[1,1′-biphenyl]-3-yl)-3-(((((R)-5-oxopyrrolidin-2-yl)methyl)amino)methyl)-4H-pyrido[1,2-a]pyrimidin-4-one;
8-(2′-chloro-2-methyl-3′-(6-methyl-5-(((((S)-5-oxopyrrolidin-2-yl)methyl)amino)methyl)pyridin-2-yl)-[1,1′-biphenyl]-3-yl)-3-(((((S)-5-oxopyrrolidin-2-yl)methyl)amino)methyl)-4H-pyrido[1,2-a]pyrimidin-4-one;
8-(2′-chloro-2-methyl-3′-(6-methyl-5-(((((R)-5-oxopyrrolidin-2-yl)methyl)amino)methyl)pyridin-2-yl)-[1,1′-biphenyl]-3-yl)-3-(((((S)-5-oxopyrrolidin-2-yl)methyl)amino)methyl)-4H-pyrido[1,2-a]pyrimidin-4-one;
8-(2′-chloro-2-methyl-3′-(6-methyl-5-(((((S)-5-oxopyrrolidin-2-yl)methyl)amino)methyl)pyridin-2-yl)-[1,1′-biphenyl]-3-yl)-3-(((((R)-5-oxopyrrolidin-2-yl)methyl)amino)methyl)-4H-pyrido[1,2-a]pyrimidin-4-one;
8-(2′-chloro-2-methyl-3′-(6-methyl-5-(((((R)-5-oxopyrrolidin-2-yl)methyl)amino)methyl)pyridin-2-yl)-[1,1′-biphenyl]-3-yl)-3-(((((R)-5-oxopyrrolidin-2-yl)methyl)amino)methyl)-4H-pyrido[1,2-a]pyrimidin-4-one;
(S)-8-(2,2′-dichloro-3′-(6-methoxy-5-((((5-oxopyrrolidin-2-yl)methyl)amino)methyl)pyridin-2-yl)-[1,1′-biphenyl]-3-yl)-3-(((2-hydroxyethyl)amino)methyl)-4H-pyrido[1,2-a]pyrimidin-4-one;
(R)-8-(2,2′-dichloro-3′-(6-methoxy-5-((((5-oxopyrrolidin-2-yl)methyl)amino)methyl)pyridin-2-yl)-[1,1′-biphenyl]-3-yl)-3-(((2-hydroxyethyl)amino)methyl)-4H-pyrido[1,2-a]pyrimidin-4-one;
(S)—N-(2-(((8-(2,2′-dichloro-3′-(6-methoxy-5-((((5-oxopyrrolidin-2-yl)methyl)amino)methyl)pyridin-2-yl)-[1,1′-biphenyl]-3-yl)-4-oxo-4H-pyrido[1,2-a]pyrimidin-3-yl)methyl)amino)ethyl)acetamide;
(R)—N-(2-(((8-(2,2′-dichloro-3′-(6-methoxy-5-((((5-oxopyrrolidin-2-yl)methyl)amino)methyl)pyridin-2-yl)-[1,1′-biphenyl]-3-yl)-4-oxo-4H-pyrido[1,2-a]pyrimidin-3-yl)methyl)amino)ethyl)acetamide;
(S)-5-((((6-(2,2′-dichloro-3′-(2-(((((S)-5-oxopyrrolidin-2-yl)methyl)amino)methyl)pyrazolo[1,5-a]pyrimidin-5-yl)-[1,1′-biphenyl]-3-yl)-2-methoxypyridin-3-yl)methyl)amino)methyl)pyrrolidin-2-one;
(R)-5-((((6-(2,2′-dichloro-3′-(2-(((((S)-5-oxopyrrolidin-2-yl)methyl)amino)methyl)pyrazolo[1,5-a]pyrimidin-5-yl)-[1,1′-biphenyl]-3-yl)-2-methoxypyridin-3-yl)methyl)amino)methyl)pyrrolidin-2-one;
(S)-5-((((6-(2,2′-dichloro-3′-(2-(((((R)-5-oxopyrrolidin-2-yl)methyl)amino)methyl)pyrazolo[1,5-a]pyrimidin-5-yl)-[1,1′-biphenyl]-3-yl)-2-methoxypyridin-3-yl)methyl)amino)methyl)pyrrolidin-2-one;
(R)-5-((((6-(2,2′-dichloro-3′-(2-(((((R)-5-oxopyrrolidin-2-yl)methyl)amino)methyl)pyrazolo[1,5-a]pyrimidin-5-yl)-[1,1′-biphenyl]-3-yl)-2-methoxypyridin-3-yl)methyl)amino)methyl)pyrrolidin-2-one;
(S)-1-((8-(2,2′-dichloro-3′-(5-((3-(hydroxymethyl)azetidin-1-yl)methyl)-6-methoxypyridin-2-yl)-[1,1′-biphenyl]-3-yl)-4-oxo-4H-pyrido[1,2-a]pyrimidin-3-yl)methyl)pyrrolidine-3-carboxylic acid;
(R)-1-((8-(2,2′-dichloro-3′-(5-((3-(hydroxymethyl)azetidin-1-yl)methyl)-6-methoxypyridin-2-yl)-[1,1′-biphenyl]-3-yl)-4-oxo-4H-pyrido[1,2-a]pyrimidin-3-yl)methyl)pyrrolidine-3-carboxylic acid;
1-((8-(2,2′-dichloro-3′-(5-((3-(hydroxymethyl)azetidin-1-yl)methyl)-6-methoxypyridin-2-yl)-[1,1′-biphenyl]-3-yl)-4-oxo-4H-pyrido[1,2-a]pyrimidin-3-yl)methyl)azetidine-3-carboxylic acid;
8-(2,2′-dichloro-3′-(6-methoxy-5-(((((S)-5-oxopyrrolidin-2-yl)methyl)amino)methyl)pyridin-2-yl)-[1,1′-biphenyl]-3-yl)-3-(((((S)-5-oxopyrrolidin-2-yl)methyl)amino)methyl)-4H-quinolizin-4-one;
8-(2,2′-dichloro-3′-(6-methoxy-5-(((((R)-5-oxopyrrolidin-2-yl)methyl)amino)methyl)pyridin-2-yl)-[1,1′-biphenyl]-3-yl)-3-(((((S)-5-oxopyrrolidin-2-yl)methyl)amino)methyl)-4H-quinolizin-4-one;
8-(2,2′-dichloro-3′-(6-methoxy-5-(((((S)-5-oxopyrrolidin-2-yl)methyl)amino)methyl)pyridin-2-yl)-[1,1′-biphenyl]-3-yl)-3-(((((R)-5-oxopyrrolidin-2-yl)methyl)amino)methyl)-4H-quinolizin-4-one;
8-(2,2′-dichloro-3′-(6-methoxy-5-(((((R)-5-oxopyrrolidin-2-yl)methyl)amino)methyl)pyridin-2-yl)-[1,1′-biphenyl]-3-yl)-3-(((((R)-5-oxopyrrolidin-2-yl)methyl)amino)methyl)-4H-quinolizin-4-one;
8-(2,2′-dichloro-5′-fluoro-3′-(6-methoxy-5-(((((S)-5-oxopyrrolidin-2-yl)methyl)amino)methyl)pyridin-2-yl)-[1,1′-biphenyl]-3-yl)-3-(((((S)-5-oxopyrrolidin-2-yl)methyl)amino)methyl)-4H-pyrido[1,2-a]pyrimidin-4-one;
8-(2,2′-dichloro-5′-fluoro-3′-(6-methoxy-5-(((((R)-5-oxopyrrolidin-2-yl)methyl)amino)methyl)pyridin-2-yl)-[1,1′-biphenyl]-3-yl)-3-(((((S)-5-oxopyrrolidin-2-yl)methyl)amino)methyl)-4H-pyrido[1,2-a]pyrimidin-4-one;
8-(2,2′-dichloro-5′-fluoro-3′-(6-methoxy-5-(((((S)-5-oxopyrrolidin-2-yl)methyl)amino)methyl)pyridin-2-yl)-[1,1′-biphenyl]-3-yl)-3-(((((R)-5-oxopyrrolidin-2-yl)methyl)amino)methyl)-4H-pyrido[1,2-a]pyrimidin-4-one;
8-(2,2′-dichloro-5′-fluoro-3′-(6-methoxy-5-(((((R)-5-oxopyrrolidin-2-yl)methyl)amino)methyl)pyridin-2-yl)-[1,1′-biphenyl]-3-yl)-3-(((((R)-5-oxopyrrolidin-2-yl)methyl)amino)methyl)-4H-pyrido[1,2-a]pyrimidin-4-one;
8-(2,2′-dichloro-4″-(((((S)-5-oxopyrrolidin-2-yl)methyl)amino)methyl)-3″-(trifluoromethoxy)-[1,1′:3′,1″-terphenyl]-3-yl)-3-(((((S)-5-oxopyrrolidin-2-yl)methyl)amino)methyl)-4H-pyrido[1,2-a]pyrimidin-4-one;
8-(2,2′-dichloro-4″-(((((R)-5-oxopyrrolidin-2-yl)methyl)amino)methyl)-3″-(trifluoromethoxy)-[1,1′:3′,1″-terphenyl]-3-yl)-3-(((((S)-5-oxopyrrolidin-2-yl)methyl)amino)methyl)-4H-pyrido[1,2-a]pyrimidin-4-one;
8-(2,2′-dichloro-4″-(((((S)-5-oxopyrrolidin-2-yl)methyl)amino)methyl)-3″-(trifluoromethoxy)-[1,1′:3′,1″-terphenyl]-3-yl)-3-(((((R)-5-oxopyrrolidin-2-yl)methyl)amino)methyl)-4H-pyrido[1,2-a]pyrimidin-4-one;
8-(2,2′-dichloro-4″-(((((R)-5-oxopyrrolidin-2-yl)methyl)amino)methyl)-3″-(trifluoromethoxy)-[1,1′:3′,1″-terphenyl]-3-yl)-3-(((((R)-5-oxopyrrolidin-2-yl)methyl)amino)methyl)-4H-pyrido[1,2-a]pyrimidin-4-one;
8-(2′-chloro-2-methyl-4″-(((((S)-5-oxopyrrolidin-2-yl)methyl)amino)methyl)-3″-(trifluoromethoxy)-[1,1′:3′,1″-terphenyl]-3-yl)-3-(((((S)-5-oxopyrrolidin-2-yl)methyl)amino)methyl)-4H-pyrido[1,2-a]pyrimidin-4-one;
8-(2′-chloro-2-methyl-4″-(((((R)-5-oxopyrrolidin-2-yl)methyl)amino)methyl)-3″-(trifluoromethoxy)-[1,1′:3′,1″-terphenyl]-3-yl)-3-(((((S)-5-oxopyrrolidin-2-yl)methyl)amino)methyl)-4H-pyrido[1,2-a]pyrimidin-4-one;
8-(2′-chloro-2-methyl-4″-(((((S)-5-oxopyrrolidin-2-yl)methyl)amino)methyl)-3″-(trifluoromethoxy)-[1,1′:3′,1″-terphenyl]-3-yl)-3-(((((R)-5-oxopyrrolidin-2-yl)methyl)amino)methyl)-4H-pyrido[1,2-a]pyrimidin-4-one;
8-(2′-chloro-2-methyl-4″-(((((R)-5-oxopyrrolidin-2-yl)methyl)amino)methyl)-3″-(trifluoromethoxy)-[1,1′:3′,1″-terphenyl]-3-yl)-3-(((((R)-5-oxopyrrolidin-2-yl)methyl)amino)methyl)-4H-pyrido[1,2-a]pyrimidin-4-one;
8-(2,2′-dichloro-3′-(6-(methylamino)-5-(((((S)-5-oxopyrrolidin-2-yl)methyl)amino)methyl)pyridin-2-yl)-[1,1′-biphenyl]-3-yl)-3-(((((S)-5-oxopyrrolidin-2-yl)methyl)amino)methyl)-4H-pyrido[1,2-a]pyrimidin-4-one;
8-(2,2′-dichloro-3′-(6-(methylamino)-5-(((((R)-5-oxopyrrolidin-2-yl)methyl)amino)methyl)pyridin-2-yl)-[1,1′-biphenyl]-3-yl)-3-(((((S)-5-oxopyrrolidin-2-yl)methyl)amino)methyl)-4H-pyrido[1,2-a]pyrimidin-4-one;
8-(2,2′-dichloro-3′-(6-(methylamino)-5-(((((S)-5-oxopyrrolidin-2-yl)methyl)amino)methyl)pyridin-2-yl)-[1,1′-biphenyl]-3-yl)-3-(((((R)-5-oxopyrrolidin-2-yl)methyl)amino)methyl)-4H-pyrido[1,2-a]pyrimidin-4-one;
8-(2,2′-dichloro-3′-(6-(methylamino)-5-(((((R)-5-oxopyrrolidin-2-yl)methyl)amino)methyl)pyridin-2-yl)-[1,1′-biphenyl]-3-yl)-3-(((((R)-5-oxopyrrolidin-2-yl)methyl)amino)methyl)-4H-pyrido[1,2-a]pyrimidin-4-one;
8-(2,2′-dichloro-3″-methoxy-4″-(((((S)-5-oxopyrrolidin-2-yl)methyl)amino)methyl)-[1,1′:3′,1″-terphenyl]-3-yl)-3-(((((S)-5-oxopyrrolidin-2-yl)methyl)amino)methyl)-4H-pyrido[1,2-a]pyrimidin-4-one;
8-(2,2′-dichloro-3″-methoxy-4″-(((((R)-5-oxopyrrolidin-2-yl)methyl)amino)methyl)-[1,1′:3′,1″-terphenyl]-3-yl)-3-(((((S)-5-oxopyrrolidin-2-yl)methyl)amino)methyl)-4H-pyrido[1,2-a]pyrimidin-4-one;
8-(2,2′-dichloro-3″-methoxy-4″-(((((S)-5-oxopyrrolidin-2-yl)methyl)amino)methyl)-[1,1′:3′,1″-terphenyl]-3-yl)-3-(((((R)-5-oxopyrrolidin-2-yl)methyl)amino)methyl)-4H-pyrido[1,2-a]pyrimidin-4-one;
8-(2,2′-dichloro-3″-methoxy-4″-(((((R)-5-oxopyrrolidin-2-yl)methyl)amino)methyl)-[1,1′:3′,1″-terphenyl]-3-yl)-3-(((((R)-5-oxopyrrolidin-2-yl)methyl)amino)methyl)-4H-pyrido[1,2-a]pyrimidin-4-one;
(S)-5-((((6-(2,2′-dichloro-3′-((S)-5-(((5-fluoropyridin-3-yl)methyl)amino)-8-methyl-5,6,7,8-tetrahydro-1,8-naphthyridin-2-yl)-[1,1′-biphenyl]-3-yl)-2-methoxypyridin-3-yl)methyl)amino)methyl)pyrrolidin-2-one;
(R)-5-((((6-(2,2′-dichloro-3′-((S)-5-(((5-fluoropyridin-3-yl)methyl)amino)-8-methyl-5,6,7,8-tetrahydro-1,8-naphthyridin-2-yl)-[1,1′-biphenyl]-3-yl)-2-methoxypyridin-3-yl)methyl)amino)methyl)pyrrolidin-2-one;
(S)-5-((((6-(2,2′-dichloro-3′-((R)-5-(((5-fluoropyridin-3-yl)methyl)amino)-8-methyl-5,6,7,8-tetrahydro-1,8-naphthyridin-2-yl)-[1,1′-biphenyl]-3-yl)-2-methoxypyridin-3-yl)methyl)amino)methyl)pyrrolidin-2-one;
(R)-5-((((6-(2,2′-dichloro-3′-((R)-5-(((5-fluoropyridin-3-yl)methyl)amino)-8-methyl-5,6,7,8-tetrahydro-1,8-naphthyridin-2-yl)-[1,1′-biphenyl]-3-yl)-2-methoxypyridin-3-yl)methyl)amino)methyl)pyrrolidin-2-one;
(S)-1-((8-(2,2′-dichloro-3′-(5-((3-(hydroxymethyl)azetidin-1-yl)methyl)-6-methoxypyrazin-2-yl)-[1,1′-biphenyl]-3-yl)-4-oxo-4H-pyrido[1,2-a]pyrimidin-3-yl)methyl)pyrrolidine-3-carboxylic acid;
(R)-1-((8-(2,2′-dichloro-3′-(5-((3-(hydroxymethyl)azetidin-1-yl)methyl)-6-methoxypyrazin-2-yl)-[1,1′-biphenyl]-3-yl)-4-oxo-4H-pyrido[1,2-a]pyrimidin-3-yl)methyl)pyrrolidine-3-carboxylic acid;
1-((8-(2,2′-dichloro-3′-(5-((3-(hydroxymethyl)azetidin-1-yl)methyl)-6-methoxypyrazin-2-yl)-[1,1′-biphenyl]-3-yl)-4-oxo-4H-pyrido[1,2-a]pyrimidin-3-yl)methyl)azetidine-3-carboxylic acid;
(S)-1-((8-(2,2′-dichloro-3′-(6-methoxy-5-(((((S)-5-oxopyrrolidin-2-yl)methyl)amino)methyl)pyridin-2-yl)-[1,1′-biphenyl]-3-yl)-4-oxo-4H-pyrido[1,2-a]pyrimidin-3-yl)methyl)pyrrolidine-3-carboxylic acid;
(R)-1-((8-(2,2′-dichloro-3′-(6-methoxy-5-(((((S)-5-oxopyrrolidin-2-yl)methyl)amino)methyl)pyridin-2-yl)-[1,1′-biphenyl]-3-yl)-4-oxo-4H-pyrido[1,2-a]pyrimidin-3-yl)methyl)pyrrolidine-3-carboxylic acid;
(S)-1-((8-(2,2′-dichloro-3′-(6-methoxy-5-(((((R)-5-oxopyrrolidin-2-yl)methyl)amino)methyl)pyridin-2-yl)-[1,1′-biphenyl]-3-yl)-4-oxo-4H-pyrido[1,2-a]pyrimidin-3-yl)methyl)pyrrolidine-3-carboxylic acid;
(R)-1-((8-(2,2′-dichloro-3′-(6-methoxy-5-(((((R)-5-oxopyrrolidin-2-yl)methyl)amino)methyl)pyridin-2-yl)-[1,1′-biphenyl]-3-yl)-4-oxo-4H-pyrido[1,2-a]pyrimidin-3-yl)methyl)pyrrolidine-3-carboxylic acid;
8-(2-chloro-3′-(6-methoxy-5-(((((S)-5-oxopyrrolidin-2-yl)methyl)amino)methyl)pyridin-2-yl)-2′-(trifluoromethyl)-[1,1′-biphenyl]-3-yl)-3-(((((S)-5-oxopyrrolidin-2-yl)methyl)amino)methyl)-4H-pyrido[1,2-a]pyrimidin-4-one;
8-(2-chloro-3′-(6-methoxy-5-(((((R)-5-oxopyrrolidin-2-yl)methyl)amino)methyl)pyridin-2-yl)-2′-(trifluoromethyl)-[1,1′-biphenyl]-3-yl)-3-(((((S)-5-oxopyrrolidin-2-yl)methyl)amino)methyl)-4H-pyrido[1,2-a]pyrimidin-4-one;
8-(2-chloro-3′-(6-methoxy-5-(((((S)-5-oxopyrrolidin-2-yl)methyl)amino)methyl)pyridin-2-yl)-2′-(trifluoromethyl)-[1,1′-biphenyl]-3-yl)-3-(((((R)-5-oxopyrrolidin-2-yl)methyl)amino)methyl)-4H-pyrido[1,2-a]pyrimidin-4-one;
8-(2-chloro-3′-(6-methoxy-5-(((((R)-5-oxopyrrolidin-2-yl)methyl)amino)methyl)pyridin-2-yl)-2′-(trifluoromethyl)-[1,1′-biphenyl]-3-yl)-3-(((((R)-5-oxopyrrolidin-2-yl)methyl)amino)methyl)-4H-pyrido[1,2-a]pyrimidin-4-one;
8-(3′-(6-methoxy-5-(((((S)-5-oxopyrrolidin-2-yl)methyl)amino)methyl)pyridin-2-yl)-2-methyl-2′-(trifluoromethyl)-[1,1′-biphenyl]-3-yl)-3-(((((S)-5-oxopyrrolidin-2-yl)methyl)amino)methyl)-4H-pyrido[1,2-a]pyrimidin-4-one;
8-(3′-(6-methoxy-5-(((((R)-5-oxopyrrolidin-2-yl)methyl)amino)methyl)pyridin-2-yl)-2-methyl-2′-(trifluoromethyl)-[1,1′-biphenyl]-3-yl)-3-(((((S)-5-oxopyrrolidin-2-yl)methyl)amino)methyl)-4H-pyrido[1,2-a]pyrimidin-4-one;
8-(3′-(6-methoxy-5-(((((S)-5-oxopyrrolidin-2-yl)methyl)amino)methyl)pyridin-2-yl)-2-methyl-2′-(trifluoromethyl)-[1,1′-biphenyl]-3-yl)-3-(((((R)-5-oxopyrrolidin-2-yl)methyl)amino)methyl)-4H-pyrido[1,2-a]pyrimidin-4-one;
8-(3′-(6-methoxy-5-(((((R)-5-oxopyrrolidin-2-yl)methyl)amino)methyl)pyridin-2-yl)-2-methyl-2′-(trifluoromethyl)-[1,1′-biphenyl]-3-yl)-3-(((((R)-5-oxopyrrolidin-2-yl)methyl)amino)methyl)-4H-pyrido[1,2-a]pyrimidin-4-one;
8-(2′-chloro-5′-fluoro-3′-(6-methoxy-5-(((((S)-5-oxopyrrolidin-2-yl)methyl)amino)methyl)pyridin-2-yl)-2-methyl-[1,1′-biphenyl]-3-yl)-3-(((((S)-5-oxopyrrolidin-2-yl)methyl)amino)methyl)-4H-pyrido[1,2-a]pyrimidin-4-one;
8-(2′-chloro-5′-fluoro-3′-(6-methoxy-5-(((((R)-5-oxopyrrolidin-2-yl)methyl)amino)methyl)pyridin-2-yl)-2-methyl-[1,1′-biphenyl]-3-yl)-3-(((((S)-5-oxopyrrolidin-2-yl)methyl)amino)methyl)-4H-pyrido[1,2-a]pyrimidin-4-one;
8-(2′-chloro-5′-fluoro-3′-(6-methoxy-5-(((((S)-5-oxopyrrolidin-2-yl)methyl)amino)methyl)pyridin-2-yl)-2-methyl-[1,1′-biphenyl]-3-yl)-3-(((((R)-5-oxopyrrolidin-2-yl)methyl)amino)methyl)-4H-pyrido[1,2-a]pyrimidin-4-one;
8-(2′-chloro-5′-fluoro-3′-(6-methoxy-5-(((((R)-5-oxopyrrolidin-2-yl)methyl)amino)methyl)pyridin-2-yl)-2-methyl-[1,1′-biphenyl]-3-yl)-3-(((((R)-5-oxopyrrolidin-2-yl)methyl)amino)methyl)-4H-pyrido[1,2-a]pyrimidin-4-one;
(S)-1-((8-(2,2′-dichloro-3′-(6-methoxy-5-((((5-oxopyrrolidin-2-yl)methyl)amino)methyl)pyridin-2-yl)-[1,1′-biphenyl]-3-yl)-4-oxo-4H-pyrido[1,2-a]pyrimidin-3-yl)methyl)azetidine-3-carboxylic acid;
(R)-1-((8-(2,2′-dichloro-3′-(6-methoxy-5-((((5-oxopyrrolidin-2-yl)methyl)amino)methyl)pyridin-2-yl)-[1,1′-biphenyl]-3-yl)-4-oxo-4H-pyrido[1,2-a]pyrimidin-3-yl)methyl)azetidine-3-carboxylic acid;
(S)-5-((((6-(2,2′-dichloro-3′-(6-(((((S)-5-oxopyrrolidin-2-yl)methyl)amino)methyl)imidazo[1,2-a]pyridin-2-yl)-[1,1′-biphenyl]-3-yl)-2-methoxypyridin-3-yl)methyl)amino)methyl)pyrrolidin-2-one;
(R)-5-((((6-(2,2′-dichloro-3′-(6-(((((S)-5-oxopyrrolidin-2-yl)methyl)amino)methyl)imidazo[1,2-a]pyridin-2-yl)-[1,1′-biphenyl]-3-yl)-2-methoxypyridin-3-yl)methyl)amino)methyl)pyrrolidin-2-one;
(S)-5-((((6-(2,2′-dichloro-3′-(6-(((((R)-5-oxopyrrolidin-2-yl)methyl)amino)methyl)imidazo[1,2-a]pyridin-2-yl)-[1,1′-biphenyl]-3-yl)-2-methoxypyridin-3-yl)methyl)amino)methyl)pyrrolidin-2-one;
(R)-5-((((6-(2,2′-dichloro-3′-(6-(((((R)-5-oxopyrrolidin-2-yl)methyl)amino)methyl)imidazo[1,2-a]pyridin-2-yl)-[1,1′-biphenyl]-3-yl)-2-methoxypyridin-3-yl)methyl)amino)methyl)pyrrolidin-2-one;
2-(((2-(2,2′-dichloro-3′-(5-(((2-hydroxyethyl)amino)methyl)-6-methoxypyridin-2-yl)-[1,1′-biphenyl]-3-yl)imidazo[1,2-a]pyridin-6-yl)methyl)amino)ethan-1-ol;
methyl ((8-(2,2′-dichloro-3′-(6-methoxy-5-(((((S)-5-oxopyrrolidin-2-yl)methyl)amino)methyl)pyridin-2-yl)-[1,1′-biphenyl]-3-yl)-4-oxo-4H-pyrido[1,2-a]pyrimidin-3-yl)methyl)-L-serinate;
methyl ((8-(2,2′-dichloro-3′-(6-methoxy-5-(((((R)-5-oxopyrrolidin-2-yl)methyl)amino)methyl)pyridin-2-yl)-[1,1′-biphenyl]-3-yl)-4-oxo-4H-pyrido[1,2-a]pyrimidin-3-yl)methyl)-L-serinate;
methyl ((8-(2,2′-dichloro-3′-(6-methoxy-5-(((((S)-5-oxopyrrolidin-2-yl)methyl)amino)methyl)pyridin-2-yl)-[1,1′-biphenyl]-3-yl)-4-oxo-4H-pyrido[1,2-a]pyrimidin-3-yl)methyl)-D-serinate;
methyl ((8-(2,2′-dichloro-3′-(6-methoxy-5-(((((R)-5-oxopyrrolidin-2-yl)methyl)amino)methyl)pyridin-2-yl)-[1,1′-biphenyl]-3-yl)-4-oxo-4H-pyrido[1,2-a]pyrimidin-3-yl)methyl)-D-serinate;
(S)-8-(2,2′-dichloro-3′-(5-(((2-hydroxyethyl)amino)methyl)-6-methoxypyridin-2-yl)-[1,1′-biphenyl]-3-yl)-3-((((5-oxopyrrolidin-2-yl)methyl)amino)methyl)-4H-pyrido[1,2-a]pyrimidin-4-one;
(R)-8-(2,2′-dichloro-3′-(5-(((2-hydroxyethyl)amino)methyl)-6-methoxypyridin-2-yl)-[1,1′-biphenyl]-3-yl)-3-((((5-oxopyrrolidin-2-yl)methyl)amino)methyl)-4H-pyrido[1,2-a]pyrimidin-4-one;
(S)—N-(2-(((6-(2,2′-dichloro-3′-(4-oxo-3-((((5-oxopyrrolidin-2-yl)methyl)amino)methyl)-4H-pyrido[1,2-a]pyrimidin-8-yl)-[1,1′-biphenyl]-3-yl)-2-methoxypyridin-3-yl)methyl)amino)ethyl)acetamide;
(R)—N-(2-(((6-(2,2′-dichloro-3′-(4-oxo-3-((((5-oxopyrrolidin-2-yl)methyl)amino)methyl)-4H-pyrido[1,2-a]pyrimidin-8-yl)-[1,1′-biphenyl]-3-yl)-2-methoxypyridin-3-yl)methyl)amino)ethyl)acetamide;
8-(2,2′-dichloro-3″-fluoro-5″-methoxy-4″-(((((S)-5-oxopyrrolidin-2-yl)methyl)amino)methyl)-[1,1′:3′,1″-terphenyl]-3-yl)-3-(((((S)-5-oxopyrrolidin-2-yl)methyl)amino)methyl)-4H-pyrido[1,2-a]pyrimidin-4-one;
8-(2,2′-dichloro-3″-fluoro-5″-methoxy-4″-(((((R)-5-oxopyrrolidin-2-yl)methyl)amino)methyl)-[1,1′:3′,1″-terphenyl]-3-yl)-3-(((((S)-5-oxopyrrolidin-2-yl)methyl)amino)methyl)-4H-pyrido[1,2-a]pyrimidin-4-one;
8-(2,2′-dichloro-3″-fluoro-5″-methoxy-4″-(((((S)-5-oxopyrrolidin-2-yl)methyl)amino)methyl)-[1,1′:3′,1″-terphenyl]-3-yl)-3-(((((R)-5-oxopyrrolidin-2-yl)methyl)amino)methyl)-4H-pyrido[1,2-a]pyrimidin-4-one;
8-(2,2′-dichloro-3″-fluoro-5″-methoxy-4″-(((((R)-5-oxopyrrolidin-2-yl)methyl)amino)methyl)-[1,1′:3′,1″-terphenyl]-3-yl)-3-(((((R)-5-oxopyrrolidin-2-yl)methyl)amino)methyl)-4H-pyrido[1,2-a]pyrimidin-4-one;
8-(2,2′-dichloro-2″-fluoro-3″-methoxy-4″-(((((S)-5-oxopyrrolidin-2-yl)methyl)amino)methyl)-[1,1′:3′,1″-terphenyl]-3-yl)-3-(((((S)-5-oxopyrrolidin-2-yl)methyl)amino)methyl)-4H-pyrido[1,2-a]pyrimidin-4-one;
8-(2,2′-dichloro-2″-fluoro-3″-methoxy-4″-(((((R)-5-oxopyrrolidin-2-yl)methyl)amino)methyl)-[1,1′:3′,1″-terphenyl]-3-yl)-3-(((((S)-5-oxopyrrolidin-2-yl)methyl)amino)methyl)-4H-pyrido[1,2-a]pyrimidin-4-one;
8-(2,2′-dichloro-2″-fluoro-3″-methoxy-4″-(((((S)-5-oxopyrrolidin-2-yl)methyl)amino)methyl)-[1,1′:3′,1″-terphenyl]-3-yl)-3-(((((R)-5-oxopyrrolidin-2-yl)methyl)amino)methyl)-4H-pyrido[1,2-a]pyrimidin-4-one;
8-(2,2′-dichloro-2″-fluoro-3″-methoxy-4″-(((((R)-5-oxopyrrolidin-2-yl)methyl)amino)methyl)-[1,1′:3′,1″-terphenyl]-3-yl)-3-(((((R)-5-oxopyrrolidin-2-yl)methyl)amino)methyl)-4H-pyrido[1,2-a]pyrimidin-4-one;
8-(2,2′-dichloro-2″-fluoro-5″-methoxy-4″-(((((S)-5-oxopyrrolidin-2-yl)methyl)amino)methyl)-[1,1′:3′,1″-terphenyl]-3-yl)-3-(((((S)-5-oxopyrrolidin-2-yl)methyl)amino)methyl)-4H-pyrido[1,2-a]pyrimidin-4-one;
8-(2,2′-dichloro-2″-fluoro-5″-methoxy-4″-(((((R)-5-oxopyrrolidin-2-yl)methyl)amino)methyl)-[1,1′:3′,1″-terphenyl]-3-yl)-3-(((((S)-5-oxopyrrolidin-2-yl)methyl)amino)methyl)-4H-pyrido[1,2-a]pyrimidin-4-one;
8-(2,2′-dichloro-2″-fluoro-5″-methoxy-4″-(((((S)-5-oxopyrrolidin-2-yl)methyl)amino)methyl)-[1,1′:3′,1″-terphenyl]-3-yl)-3-(((((R)-5-oxopyrrolidin-2-yl)methyl)amino)methyl)-4H-pyrido[1,2-a]pyrimidin-4-one;
8-(2,2′-dichloro-2″-fluoro-5″-methoxy-4″-(((((R)-5-oxopyrrolidin-2-yl)methyl)amino)methyl)-[1,1′:3′,1″-terphenyl]-3-yl)-3-(((((R)-5-oxopyrrolidin-2-yl)methyl)amino)methyl)-4H-pyrido[1,2-a]pyrimidin-4-one;
8-(2,2′-dichloro-3′-(6-methoxy-5-(((((S)-5-oxopyrrolidin-2-yl)methyl)amino)methyl)pyrazin-2-yl)-[1,1′-biphenyl]-3-yl)-3-(((((S)-5-oxopyrrolidin-2-yl)methyl)amino)methyl)-4H-quinolizin-4-one;
8-(2,2′-dichloro-3′-(6-methoxy-5-(((((R)-5-oxopyrrolidin-2-yl)methyl)amino)methyl)pyrazin-2-yl)-[1,1′-biphenyl]-3-yl)-3-(((((S)-5-oxopyrrolidin-2-yl)methyl)amino)methyl)-4H-quinolizin-4-one;
8-(2,2′-dichloro-3′-(6-methoxy-5-(((((S)-5-oxopyrrolidin-2-yl)methyl)amino)methyl)pyrazin-2-yl)-[1,1′-biphenyl]-3-yl)-3-(((((R)-5-oxopyrrolidin-2-yl)methyl)amino)methyl)-4H-quinolizin-4-one;
8-(2,2′-dichloro-3′-(6-methoxy-5-(((((R)-5-oxopyrrolidin-2-yl)methyl)amino)methyl)pyrazin-2-yl)-[1,1′-biphenyl]-3-yl)-3-(((((R)-5-oxopyrrolidin-2-yl)methyl)amino)methyl)-4H-quinolizin-4-one;
8-(2,2′-dichloro-3′-(6-(methylthio)-5-(((((S)-5-oxopyrrolidin-2-yl)methyl)amino)methyl)pyridin-2-yl)-[1,1′-biphenyl]-3-yl)-3-(((((S)-5-oxopyrrolidin-2-yl)methyl)amino)methyl)-4H-pyrido[1,2-a]pyrimidin-4-one;
8-(2,2′-dichloro-3′-(6-(methylthio)-5-(((((R)-5-oxopyrrolidin-2-yl)methyl)amino)methyl)pyridin-2-yl)-[1,1′-biphenyl]-3-yl)-3-(((((S)-5-oxopyrrolidin-2-yl)methyl)amino)methyl)-4H-pyrido[1,2-a]pyrimidin-4-one;
8-(2,2′-dichloro-3′-(6-(methylthio)-5-(((((S)-5-oxopyrrolidin-2-yl)methyl)amino)methyl)pyridin-2-yl)-[1,1′-biphenyl]-3-yl)-3-(((((R)-5-oxopyrrolidin-2-yl)methyl)amino)methyl)-4H-pyrido[1,2-a]pyrimidin-4-one;
8-(2,2′-dichloro-3′-(6-(methylthio)-5-(((((R)-5-oxopyrrolidin-2-yl)methyl)amino)methyl)pyridin-2-yl)-[1,1′-biphenyl]-3-yl)-3-(((((R)-5-oxopyrrolidin-2-yl)methyl)amino)methyl)-4H-pyrido[1,2-a]pyrimidin-4-one;
8-(2,2′-dichloro-3′-(6-cyclopropoxy-5-(((((S)-5-oxopyrrolidin-2-yl)methyl)amino)methyl)pyridin-2-yl)-[1,1′-biphenyl]-3-yl)-3-(((((S)-5-oxopyrrolidin-2-yl)methyl)amino)methyl)-4H-pyrido[1,2-a]pyrimidin-4-one;
8-(2,2′-dichloro-3′-(6-cyclopropoxy-5-(((((R)-5-oxopyrrolidin-2-yl)methyl)amino)methyl)pyridin-2-yl)-[1,1′-biphenyl]-3-yl)-3-(((((S)-5-oxopyrrolidin-2-yl)methyl)amino)methyl)-4H-pyrido[1,2-a]pyrimidin-4-one;
8-(2,2′-dichloro-3′-(6-cyclopropoxy-5-(((((S)-5-oxopyrrolidin-2-yl)methyl)amino)methyl)pyridin-2-yl)-[1,1′-biphenyl]-3-yl)-3-(((((R)-5-oxopyrrolidin-2-yl)methyl)amino)methyl)-4H-pyrido[1,2-a]pyrimidin-4-one;
8-(2,2′-dichloro-3′-(6-cyclopropoxy-5-(((((R)-5-oxopyrrolidin-2-yl)methyl)amino)methyl)pyridin-2-yl)-[1,1′-biphenyl]-3-yl)-3-(((((R)-5-oxopyrrolidin-2-yl)methyl)amino)methyl)-4H-pyrido[1,2-a]pyrimidin-4-one;
8-(2,2′-dichloro-3″-(difluoromethoxy)-4″-(((((S)-5-oxopyrrolidin-2-yl)methyl)amino)methyl)-[1,1′:3′,1″-terphenyl]-3-yl)-3-(((((S)-5-oxopyrrolidin-2-yl)methyl)amino)methyl)-4H-pyrido[1,2-a]pyrimidin-4-one;
8-(2,2′-dichloro-3″-(difluoromethoxy)-4″-(((((R)-5-oxopyrrolidin-2-yl)methyl)amino)methyl)-[1,1′:3′,1″-terphenyl]-3-yl)-3-(((((S)-5-oxopyrrolidin-2-yl)methyl)amino)methyl)-4H-pyrido[1,2-a]pyrimidin-4-one;
8-(2,2′-dichloro-3″-(difluoromethoxy)-4″-(((((S)-5-oxopyrrolidin-2-yl)methyl)amino)methyl)-[1,1′:3′,1″-terphenyl]-3-yl)-3-(((((R)-5-oxopyrrolidin-2-yl)methyl)amino)methyl)-4H-pyrido[1,2-a]pyrimidin-4-one;
8-(2,2′-dichloro-3″-(difluoromethoxy)-4″-(((((R)-5-oxopyrrolidin-2-yl)methyl)amino)methyl)-[1,1′:3′,1″-terphenyl]-3-yl)-3-(((((R)-5-oxopyrrolidin-2-yl)methyl)amino)methyl)-4H-pyrido[1,2-a]pyrimidin-4-one;
(S)-8-(2,2′-dichloro-3′-(6-methoxy-5-((((5-oxopyrrolidin-2-yl)methyl)amino)methyl)pyridin-2-yl)-[1,1′-biphenyl]-3-yl)-3-((3-(2-hydroxypropan-2-yl)azetidin-1-yl)methyl)-4H-pyrido[1,2-a]pyrimidin-4-one;
(R)-8-(2,2′-dichloro-3′-(6-methoxy-5-((((5-oxopyrrolidin-2-yl)methyl)amino)methyl)pyridin-2-yl)-[1,1′-biphenyl]-3-yl)-3-((3-(2-hydroxypropan-2-yl)azetidin-1-yl)methyl)-4H-pyrido[1,2-a]pyrimidin-4-one;
(S)-8-(2,2′-dichloro-3′-(5-((3-(2-hydroxypropan-2-yl)azetidin-1-yl)methyl)-6-methoxypyridin-2-yl)-[1,1′-biphenyl]-3-yl)-3-((((5-oxopyrrolidin-2-yl)methyl)amino)methyl)-4H-pyrido[1,2-a]pyrimidin-4-one;
(R)-8-(2,2′-dichloro-3′-(5-((3-(2-hydroxypropan-2-yl)azetidin-1-yl)methyl)-6-methoxypyridin-2-yl)-[1,1′-biphenyl]-3-yl)-3-((((5-oxopyrrolidin-2-yl)methyl)amino)methyl)-4H-pyrido[1,2-a]pyrimidin-4-one;
methyl ((6-(2,2′-dichloro-3′-(4-oxo-3-(((((S)-5-oxopyrrolidin-2-yl)methyl)amino)methyl)-4H-pyrido[1,2-a]pyrimidin-8-yl)-[1,1′-biphenyl]-3-yl)-2-methoxypyridin-3-yl)methyl)-L-serinate;
methyl ((6-(2,2′-dichloro-3′-(4-oxo-3-(((((R)-5-oxopyrrolidin-2-yl)methyl)amino)methyl)-4H-pyrido[1,2-a]pyrimidin-8-yl)-[1,1′-biphenyl]-3-yl)-2-methoxypyridin-3-yl)methyl)-L-serinate;
methyl ((6-(2,2′-dichloro-3′-(4-oxo-3-(((((S)-5-oxopyrrolidin-2-yl)methyl)amino)methyl)-4H-pyrido[1,2-a]pyrimidin-8-yl)-[1,1′-biphenyl]-3-yl)-2-methoxypyridin-3-yl)methyl)-D-serinate;
methyl ((6-(2,2′-dichloro-3′-(4-oxo-3-(((((R)-5-oxopyrrolidin-2-yl)methyl)amino)methyl)-4H-pyrido[1,2-a]pyrimidin-8-yl)-[1,1′-biphenyl]-3-yl)-2-methoxypyridin-3-yl)methyl)-D-serinate;
((6-(2,2′-dichloro-3′-(4-oxo-3-(((((S)-5-oxopyrrolidin-2-yl)methyl)amino)methyl)-4H-pyrido[1,2-a]pyrimidin-8-yl)-[1,1′-biphenyl]-3-yl)-2-methoxypyridin-3-yl)methyl)-L-serine;
((6-(2,2′-dichloro-3′-(4-oxo-3-(((((R)-5-oxopyrrolidin-2-yl)methyl)amino)methyl)-4H-pyrido[1,2-a]pyrimidin-8-yl)-[1,1′-biphenyl]-3-yl)-2-methoxypyridin-3-yl)methyl)-L-serine;
((6-(2,2′-dichloro-3′-(4-oxo-3-(((((S)-5-oxopyrrolidin-2-yl)methyl)amino)methyl)-4H-pyrido[1,2-a]pyrimidin-8-yl)-[1,1′-biphenyl]-3-yl)-2-methoxypyridin-3-yl)methyl)-D-serine;
((6-(2,2′-dichloro-3′-(4-oxo-3-(((((R)-5-oxopyrrolidin-2-yl)methyl)amino)methyl)-4H-pyrido[1,2-a]pyrimidin-8-yl)-[1,1′-biphenyl]-3-yl)-2-methoxypyridin-3-yl)methyl)-D-serine;
(S)-1-(((6-(2,2′-dichloro-3′-(4-oxo-3-((((5-oxopyrrolidin-2-yl)methyl)amino)methyl)-4H-pyrido[1,2-a]pyrimidin-8-yl)-[1,1′-biphenyl]-3-yl)-2-methoxypyridin-3-yl)methyl)amino)cyclopropane-1-carboxylic acid;
(R)-1-(((6-(2,2′-dichloro-3′-(4-oxo-3-((((5-oxopyrrolidin-2-yl)methyl)amino)methyl)-4H-pyrido[1,2-a]pyrimidin-8-yl)-[1,1′-biphenyl]-3-yl)-2-methoxypyridin-3-yl)methyl)amino)cyclopropane-1-carboxylic acid;
(S)-1-((8-(2,2′-dichloro-3′-(6-methoxy-5-(((((S)-5-oxopyrrolidin-2-yl)methyl)amino)methyl)pyrazin-2-yl)-[1,1′-biphenyl]-3-yl)-4-oxo-4H-pyrido[1,2-a]pyrimidin-3-yl)methyl)pyrrolidine-3-carboxylic acid;
(R)-1-((8-(2,2′-dichloro-3′-(6-methoxy-5-(((((S)-5-oxopyrrolidin-2-yl)methyl)amino)methyl)pyrazin-2-yl)-[1,1′-biphenyl]-3-yl)-4-oxo-4H-pyrido[1,2-a]pyrimidin-3-yl)methyl)pyrrolidine-3-carboxylic acid;
(S)-1-((8-(2,2′-dichloro-3′-(6-methoxy-5-(((((R)-5-oxopyrrolidin-2-yl)methyl)amino)methyl)pyrazin-2-yl)-[1,1′-biphenyl]-3-yl)-4-oxo-4H-pyrido[1,2-a]pyrimidin-3-yl)methyl)pyrrolidine-3-carboxylic acid;
(R)-1-((8-(2,2′-dichloro-3′-(6-methoxy-5-(((((R)-5-oxopyrrolidin-2-yl)methyl)amino)methyl)pyrazin-2-yl)-[1,1′-biphenyl]-3-yl)-4-oxo-4H-pyrido[1,2-a]pyrimidin-3-yl)methyl)pyrrolidine-3-carboxylic acid;
(S)-1-((8-(2,2′-dichloro-3′-(6-methoxy-5-((((5-oxopyrrolidin-2-yl)methyl)amino)methyl)pyrazin-2-yl)-[1,1′-biphenyl]-3-yl)-4-oxo-4H-pyrido[1,2-a]pyrimidin-3-yl)methyl)azetidine-3-carboxylic acid;
(R)-1-((8-(2,2′-dichloro-3′-(6-methoxy-5-((((5-oxopyrrolidin-2-yl)methyl)amino)methyl)pyrazin-2-yl)-[1,1′-biphenyl]-3-yl)-4-oxo-4H-pyrido[1,2-a]pyrimidin-3-yl)methyl)azetidine-3-carboxylic acid;
(S)-1-((6-(2,2′-dichloro-3′-(4-oxo-3-(((((S)-5-oxopyrrolidin-2-yl)methyl)amino)methyl)-4H-pyrido[1,2-a]pyrimidin-8-yl)-[1,1′-biphenyl]-3-yl)-2-methoxypyridin-3-yl)methyl)pyrrolidine-3-carboxylic acid;
(R)-1-((6-(2,2′-dichloro-3′-(4-oxo-3-(((((S)-5-oxopyrrolidin-2-yl)methyl)amino)methyl)-4H-pyrido[1,2-a]pyrimidin-8-yl)-[1,1′-biphenyl]-3-yl)-2-methoxypyridin-3-yl)methyl)pyrrolidine-3-carboxylic acid;
(S)-1-((6-(2,2′-dichloro-3′-(4-oxo-3-(((((R)-5-oxopyrrolidin-2-yl)methyl)amino)methyl)-4H-pyrido[1,2-a]pyrimidin-8-yl)-[1,1′-biphenyl]-3-yl)-2-methoxypyridin-3-yl)methyl)pyrrolidine-3-carboxylic acid;
(R)-1-((6-(2,2′-dichloro-3′-(4-oxo-3-(((((R)-5-oxopyrrolidin-2-yl)methyl)amino)methyl)-4H-pyrido[1,2-a]pyrimidin-8-yl)-[1,1′-biphenyl]-3-yl)-2-methoxypyridin-3-yl)methyl)pyrrolidine-3-carboxylic acid;
(S)-1-((6-(2,2′-dichloro-3′-(4-oxo-3-((((5-oxopyrrolidin-2-yl)methyl)amino)methyl)-4H-pyrido[1,2-a]pyrimidin-8-yl)-[1,1′-biphenyl]-3-yl)-2-methoxypyridin-3-yl)methyl)azetidine-3-carboxylic acid;
(R)-1-((6-(2,2′-dichloro-3′-(4-oxo-3-((((5-oxopyrrolidin-2-yl)methyl)amino)methyl)-4H-pyrido[1,2-a]pyrimidin-8-yl)-[1,1′-biphenyl]-3-yl)-2-methoxypyridin-3-yl)methyl)azetidine-3-carboxylic acid;
(S)-1-(((8-(2,2′-dichloro-3′-(6-methoxy-5-((((5-oxopyrrolidin-2-yl)methyl)amino)methyl)pyridin-2-yl)-[1,1′-biphenyl]-3-yl)-4-oxo-4H-pyrido[1,2-a]pyrimidin-3-yl)methyl)amino)cyclopropane-1-carboxylic acid;
(R)-1-(((8-(2,2′-dichloro-3′-(6-methoxy-5-((((5-oxopyrrolidin-2-yl)methyl)amino)methyl)pyridin-2-yl)-[1,1′-biphenyl]-3-yl)-4-oxo-4H-pyrido[1,2-a]pyrimidin-3-yl)methyl)amino)cyclopropane-1-carboxylic acid;
methyl (S)-2-((6-(2,2′-dichloro-3′-(4-oxo-3-((((5-oxopyrrolidin-2-yl)methyl)amino)methyl)-4H-pyrido[1,2-a]pyrimidin-8-yl)-[1,1′-biphenyl]-3-yl)-2-methoxypyridin-3-yl)methyl)-2-azabicyclo[2.2.2]octane-4-carboxylate;
methyl (R)-2-((6-(2,2′-dichloro-3′-(4-oxo-3-((((5-oxopyrrolidin-2-yl)methyl)amino)methyl)-4H-pyrido[1,2-a]pyrimidin-8-yl)-[1,1′-biphenyl]-3-yl)-2-methoxypyridin-3-yl)methyl)-2-azabicyclo[2.2.2]octane-4-carboxylate;
(S)-2-((6-(2,2′-dichloro-3′-(4-oxo-3-((((5-oxopyrrolidin-2-yl)methyl)amino)methyl)-4H-pyrido[1,2-a]pyrimidin-8-yl)-[1,1′-biphenyl]-3-yl)-2-methoxypyridin-3-yl)methyl)-2-azabicyclo[2.2.2]octane-4-carboxylic acid;
(R)-2-((6-(2,2′-dichloro-3′-(4-oxo-3-((((5-oxopyrrolidin-2-yl)methyl)amino)methyl)-4H-pyrido[1,2-a]pyrimidin-8-yl)-[1,1′-biphenyl]-3-yl)-2-methoxypyridin-3-yl)methyl)-2-azabicyclo[2.2.2]octane-4-carboxylic acid;
(S)-5-((((3-chloro-6-(2,2′-dichloro-3′-(6-methoxy-5-(((((S)-5-oxopyrrolidin-2-yl)methyl)amino)methyl)pyridin-2-yl)-[1,1′-biphenyl]-3-yl)thieno[3,2-b]pyridin-2-yl)methyl)amino)methyl)pyrrolidin-2-one;
(R)-5-((((3-chloro-6-(2,2′-dichloro-3′-(6-methoxy-5-(((((S)-5-oxopyrrolidin-2-yl)methyl)amino)methyl)pyridin-2-yl)-[1,1′-biphenyl]-3-yl)thieno[3,2-b]pyridin-2-yl)methyl)amino)methyl)pyrrolidin-2-one;
(S)-5-((((3-chloro-6-(2,2′-dichloro-3′-(6-methoxy-5-(((((R)-5-oxopyrrolidin-2-yl)methyl)amino)methyl)pyridin-2-yl)-[1,1′-biphenyl]-3-yl)thieno[3,2-b]pyridin-2-yl)methyl)amino)methyl)pyrrolidin-2-one;
(R)-5-((((3-chloro-6-(2,2′-dichloro-3′-(6-methoxy-5-(((((R)-5-oxopyrrolidin-2-yl)methyl)amino)methyl)pyridin-2-yl)-[1,1′-biphenyl]-3-yl)thieno[3,2-b]pyridin-2-yl)methyl)amino)methyl)pyrrolidin-2-one;
methyl (S)-((6-(2,2′-dichloro-3′-(4-oxo-3-((((5-oxopyrrolidin-2-yl)methyl)amino)methyl)-4H-pyrido[1,2-a]pyrimidin-8-yl)-[1,1′-biphenyl]-3-yl)-2-methoxypyridin-3-yl)methyl)glycinate;
methyl (R)-((6-(2,2′-dichloro-3′-(4-oxo-3-((((5-oxopyrrolidin-2-yl)methyl)amino)methyl)-4H-pyrido[1,2-a]pyrimidin-8-yl)-[1,1′-biphenyl]-3-yl)-2-methoxypyridin-3-yl)methyl)glycinate;
(S)-((6-(2,2′-dichloro-3′-(4-oxo-3-((((5-oxopyrrolidin-2-yl)methyl)amino)methyl)-4H-pyrido[1,2-a]pyrimidin-8-yl)-[1,1′-biphenyl]-3-yl)-2-methoxypyridin-3-yl)methyl)glycine;
(R)-((6-(2,2′-dichloro-3′-(4-oxo-3-((((5-oxopyrrolidin-2-yl)methyl)amino)methyl)-4H-pyrido[1,2-a]pyrimidin-8-yl)-[1,1′-biphenyl]-3-yl)-2-methoxypyridin-3-yl)methyl)glycine;
(S)-4-(((8-(2,2′-dichloro-3′-(6-methoxy-5-(((((S)-5-oxopyrrolidin-2-yl)methyl)amino)methyl)pyridin-2-yl)-[1,1′-biphenyl]-3-yl)-4-oxo-4H-pyrido[1,2-a]pyrimidin-3-yl)methyl)amino)-3-hydroxybutanoic acid;
(R)-4-(((8-(2,2′-dichloro-3′-(6-methoxy-5-(((((S)-5-oxopyrrolidin-2-yl)methyl)amino)methyl)pyridin-2-yl)-[1,1′-biphenyl]-3-yl)-4-oxo-4H-pyrido[1,2-a]pyrimidin-3-yl)methyl)amino)-3-hydroxybutanoic acid;
(S)-4-(((8-(2,2′-dichloro-3′-(6-methoxy-5-(((((R)-5-oxopyrrolidin-2-yl)methyl)amino)methyl)pyridin-2-yl)-[1,1′-biphenyl]-3-yl)-4-oxo-4H-pyrido[1,2-a]pyrimidin-3-yl)methyl)amino)-3-hydroxybutanoic acid;
(R)-4-(((8-(2,2′-dichloro-3′-(6-methoxy-5-(((((R)-5-oxopyrrolidin-2-yl)methyl)amino)methyl)pyridin-2-yl)-[1,1′-biphenyl]-3-yl)-4-oxo-4H-pyrido[1,2-a]pyrimidin-3-yl)methyl)amino)-3-hydroxybutanoic acid;
(S)-2-((8-(2,2′-dichloro-3′-(6-methoxy-5-((((5-oxopyrrolidin-2-yl)methyl)amino)methyl)pyridin-2-yl)-[1,1′-biphenyl]-3-yl)-4-oxo-4H-pyrido[1,2-a]pyrimidin-3-yl)methyl)-2-azabicyclo[2.2.2]octane-4-carboxylic acid;
(R)-2-((8-(2,2′-dichloro-3′-(6-methoxy-5-((((5-oxopyrrolidin-2-yl)methyl)amino)methyl)pyridin-2-yl)-[1,1′-biphenyl]-3-yl)-4-oxo-4H-pyrido[1,2-a]pyrimidin-3-yl)methyl)-2-azabicyclo[2.2.2]octane-4-carboxylic acid;
((8-(2,2′-dichloro-3′-(6-methoxy-5-(((((S)-5-oxopyrrolidin-2-yl)methyl)amino)methyl)pyridin-2-yl)-[1,1′-biphenyl]-3-yl)-4-oxo-4H-pyrido[1,2-a]pyrimidin-3-yl)methyl)-L-proline;
((8-(2,2′-dichloro-3′-(6-methoxy-5-(((((R)-5-oxopyrrolidin-2-yl)methyl)amino)methyl)pyridin-2-yl)-[1,1′-biphenyl]-3-yl)-4-oxo-4H-pyrido[1,2-a]pyrimidin-3-yl)methyl)-D-proline;
((8-(2,2′-dichloro-3′-(6-methoxy-5-(((((S)-5-oxopyrrolidin-2-yl)methyl)amino)methyl)pyridin-2-yl)-[1,1′-biphenyl]-3-yl)-4-oxo-4H-pyrido[1,2-a]pyrimidin-3-yl)methyl)-D-proline;
((8-(2,2′-dichloro-3′-(6-methoxy-5-(((((R)-5-oxopyrrolidin-2-yl)methyl)amino)methyl)pyridin-2-yl)-[1,1′-biphenyl]-3-yl)-4-oxo-4H-pyrido[1,2-a]pyrimidin-3-yl)methyl)-L-proline;
(S)-1-((8-(2,2′-dichloro-3′-(6-methoxy-5-(((((S)-5-oxopyrrolidin-2-yl)methyl)amino)methyl)pyridin-2-yl)-[1,1′-biphenyl]-3-yl)-4-oxo-4H-pyrido[1,2-a]pyrimidin-3-yl)methyl)piperidine-2-carboxylic acid;
(R)-1-((8-(2,2′-dichloro-3′-(6-methoxy-5-(((((S)-5-oxopyrrolidin-2-yl)methyl)amino)methyl)pyridin-2-yl)-[1,1′-biphenyl]-3-yl)-4-oxo-4H-pyrido[1,2-a]pyrimidin-3-yl)methyl)piperidine-2-carboxylic acid;
(S)-1-((8-(2,2′-dichloro-3′-(6-methoxy-5-(((((R)-5-oxopyrrolidin-2-yl)methyl)amino)methyl)pyridin-2-yl)-[1,1′-biphenyl]-3-yl)-4-oxo-4H-pyrido[1,2-a]pyrimidin-3-yl)methyl)piperidine-2-carboxylic acid;
(R)-1-((8-(2,2′-dichloro-3′-(6-methoxy-5-(((((R)-5-oxopyrrolidin-2-yl)methyl)amino)methyl)pyridin-2-yl)-[1,1′-biphenyl]-3-yl)-4-oxo-4H-pyrido[1,2-a]pyrimidin-3-yl)methyl)piperidine-2-carboxylic acid;
(S)-1-((8-(2,2′-dichloro-3′-(6-methoxy-5-(((((S)-5-oxopyrrolidin-2-yl)methyl)amino)methyl)pyridin-2-yl)-[1,1′-biphenyl]-3-yl)-4-oxo-4H-pyrido[1,2-a]pyrimidin-3-yl)methyl)piperidine-3-carboxylic acid;
(R)-1-((8-(2,2′-dichloro-3′-(6-methoxy-5-(((((S)-5-oxopyrrolidin-2-yl)methyl)amino)methyl)pyridin-2-yl)-[1,1′-biphenyl]-3-yl)-4-oxo-4H-pyrido[1,2-a]pyrimidin-3-yl)methyl)piperidine-3-carboxylic acid;
(S)-1-((8-(2,2′-dichloro-3′-(6-methoxy-5-(((((R)-5-oxopyrrolidin-2-yl)methyl)amino)methyl)pyridin-2-yl)-[1,1′-biphenyl]-3-yl)-4-oxo-4H-pyrido[1,2-a]pyrimidin-3-yl)methyl)piperidine-3-carboxylic acid;
(R)-1-((8-(2,2′-dichloro-3′-(6-methoxy-5-(((((R)-5-oxopyrrolidin-2-yl)methyl)amino)methyl)pyridin-2-yl)-[1,1′-biphenyl]-3-yl)-4-oxo-4H-pyrido[1,2-a]pyrimidin-3-yl)methyl)piperidine-3-carboxylic acid;
8-(2,2′-dichloro-3′-(6-methoxy-5-(((((S)-5-oxopyrrolidin-2-yl)methyl)amino)methyl)pyridin-2-yl)-[1,1′-biphenyl]-3-yl)-3-((((S)-2-hydroxypropyl)amino)methyl)-4H-pyrido[1,2-a]pyrimidin-4-one;
8-(2,2′-dichloro-3′-(6-methoxy-5-(((((R)-5-oxopyrrolidin-2-yl)methyl)amino)methyl)pyridin-2-yl)-[1,1′-biphenyl]-3-yl)-3-((((S)-2-hydroxypropyl)amino)methyl)-4H-pyrido[1,2-a]pyrimidin-4-one;
8-(2,2′-dichloro-3′-(6-methoxy-5-(((((S)-5-oxopyrrolidin-2-yl)methyl)amino)methyl)pyridin-2-yl)-[1,1′-biphenyl]-3-yl)-3-((((R)-2-hydroxypropyl)amino)methyl)-4H-pyrido[1,2-a]pyrimidin-4-one;
8-(2,2′-dichloro-3′-(6-methoxy-5-(((((R)-5-oxopyrrolidin-2-yl)methyl)amino)methyl)pyridin-2-yl)-[1,1′-biphenyl]-3-yl)-3-((((R)-2-hydroxypropyl)amino)methyl)-4H-pyrido[1,2-a]pyrimidin-4-one;
8-(2,2′-dichloro-3′-(6-methoxy-5-(((((S)-5-oxopyrrolidin-2-yl)methyl)amino)methyl)pyridin-2-yl)-[1,1′-biphenyl]-3-yl)-3-((((R)-2-hydroxypropyl)amino)methyl)-4H-pyrido[1,2-a]pyrimidin-4-one;
8-(2,2′-dichloro-3′-(6-methoxy-5-(((((R)-5-oxopyrrolidin-2-yl)methyl)amino)methyl)pyridin-2-yl)-[1,1′-biphenyl]-3-yl)-3-((((R)-2-hydroxypropyl)amino)methyl)-4H-pyrido[1,2-a]pyrimidin-4-one;
8-(2,2′-dichloro-3′-(6-methoxy-5-(((((S)-5-oxopyrrolidin-2-yl)methyl)amino)methyl)pyridin-2-yl)-[1,1′-biphenyl]-3-yl)-3-((((S)-2-hydroxypropyl)amino)methyl)-4H-pyrido[1,2-a]pyrimidin-4-one;
8-(2,2′-dichloro-3′-(6-methoxy-5-(((((R)-5-oxopyrrolidin-2-yl)methyl)amino)methyl)pyridin-2-yl)-[1,1′-biphenyl]-3-yl)-3-((((S)-2-hydroxypropyl)amino)methyl)-4H-pyrido[1,2-a]pyrimidin-4-one;
(S)-8-(2,2′-dichloro-3′-(6-methoxy-5-((((5-oxopyrrolidin-2-yl)methyl)amino)methyl)pyridin-2-yl)-[1,1′-biphenyl]-3-yl)-3-(((2-hydroxy-2-methylpropyl)amino)methyl)-4H-pyrido[1,2-a]pyrimidin-4-one;
(R)-8-(2,2′-dichloro-3′-(6-methoxy-5-((((5-oxopyrrolidin-2-yl)methyl)amino)methyl)pyridin-2-yl)-[1,1′-biphenyl]-3-yl)-3-(((2-hydroxy-2-methylpropyl)amino)methyl)-4H-pyrido[1,2-a]pyrimidin-4-one;
(2R,4R)-1-((8-(2,2′-dichloro-3′-(6-methoxy-5-(((((S)-5-oxopyrrolidin-2-yl)methyl)amino)methyl)pyridin-2-yl)-[1,1′-biphenyl]-3-yl)-4-oxo-4H-pyrido[1,2-a]pyrimidin-3-yl)methyl)-4-hydroxypyrrolidine-2-carboxylic acid;
(2R,4S)-1-((8-(2,2′-dichloro-3′-(6-methoxy-5-(((((S)-5-oxopyrrolidin-2-yl)methyl)amino)methyl)pyridin-2-yl)-[1,1′-biphenyl]-3-yl)-4-oxo-4H-pyrido[1,2-a]pyrimidin-3-yl)methyl)-4-hydroxypyrrolidine-2-carboxylic acid;
(2S,4R)-1-((8-(2,2′-dichloro-3′-(6-methoxy-5-(((((S)-5-oxopyrrolidin-2-yl)methyl)amino)methyl)pyridin-2-yl)-[1,1′-biphenyl]-3-yl)-4-oxo-4H-pyrido[1,2-a]pyrimidin-3-yl)methyl)-4-hydroxypyrrolidine-2-carboxylic acid;
(2S,4S)-1-((8-(2,2′-dichloro-3′-(6-methoxy-5-(((((S)-5-oxopyrrolidin-2-yl)methyl)amino)methyl)pyridin-2-yl)-[1,1′-biphenyl]-3-yl)-4-oxo-4H-pyrido[1,2-a]pyrimidin-3-yl)methyl)-4-hydroxypyrrolidine-2-carboxylic acid;
(2R,4R)-1-((8-(2,2′-dichloro-3′-(6-methoxy-5-(((((R)-5-oxopyrrolidin-2-yl)methyl)amino)methyl)pyridin-2-yl)-[1,1′-biphenyl]-3-yl)-4-oxo-4H-pyrido[1,2-a]pyrimidin-3-yl)methyl)-4-hydroxypyrrolidine-2-carboxylic acid;
(2R,4S)-1-((8-(2,2′-dichloro-3′-(6-methoxy-5-(((((R)-5-oxopyrrolidin-2-yl)methyl)amino)methyl)pyridin-2-yl)-[1,1′-biphenyl]-3-yl)-4-oxo-4H-pyrido[1,2-a]pyrimidin-3-yl)methyl)-4-hydroxypyrrolidine-2-carboxylic acid;
(2S,4R)-1-((8-(2,2′-dichloro-3′-(6-methoxy-5-(((((R)-5-oxopyrrolidin-2-yl)methyl)amino)methyl)pyridin-2-yl)-[1,1′-biphenyl]-3-yl)-4-oxo-4H-pyrido[1,2-a]pyrimidin-3-yl)methyl)-4-hydroxypyrrolidine-2-carboxylic acid;
(2S,4S)-1-((8-(2,2′-dichloro-3′-(6-methoxy-5-(((((R)-5-oxopyrrolidin-2-yl)methyl)amino)methyl)pyridin-2-yl)-[1,1′-biphenyl]-3-yl)-4-oxo-4H-pyrido[1,2-a]pyrimidin-3-yl)methyl)-4-hydroxypyrrolidine-2-carboxylic acid;
(S)-1-((6-(2,2′-dichloro-3′-(3-((3-(hydroxymethyl)azetidin-1-yl)methyl)-4-oxo-4H-pyrido[1,2-a]pyrimidin-8-yl)-[1,1′-biphenyl]-3-yl)-2-methoxypyridin-3-yl)methyl)pyrrolidine-3-carboxylic acid;
(R)-1-((6-(2,2′-dichloro-3′-(3-((3-(hydroxymethyl)azetidin-1-yl)methyl)-4-oxo-4H-pyrido[1,2-a]pyrimidin-8-yl)-[1,1′-biphenyl]-3-yl)-2-methoxypyridin-3-yl)methyl)pyrrolidine-3-carboxylic acid;
1-((6-(2,2′-dichloro-3′-(3-((3-(hydroxymethyl)azetidin-1-yl)methyl)-4-oxo-4H-pyrido[1,2-a]pyrimidin-8-yl)-[1,1′-biphenyl]-3-yl)-2-methoxypyridin-3-yl)methyl)azetidine-3-carboxylic acid;
methyl (S)-4-(((8-(2,2′-dichloro-3′-(6-methoxy-5-((((5-oxopyrrolidin-2-yl)methyl)amino)methyl)pyridin-2-yl)-[1,1′-biphenyl]-3-yl)-4-oxo-4H-pyrido[1,2-a]pyrimidin-3-yl)methyl)amino)butanoate;
methyl (R)-4-(((8-(2,2′-dichloro-3′-(6-methoxy-5-((((5-oxopyrrolidin-2-yl)methyl)amino)methyl)pyridin-2-yl)-[1,1′-biphenyl]-3-yl)-4-oxo-4H-pyrido[1,2-a]pyrimidin-3-yl)methyl)amino)butanoate;
(S)-8-(2,2′-dichloro-3′-(6-methoxy-5-((((5-oxopyrrolidin-2-yl)methyl)amino)methyl)pyridin-2-yl)-[1,1′-biphenyl]-3-yl)-3-(((2,4-dimethoxybenzyl)amino)methyl)-4H-pyrido[1,2-a]pyrimidin-4-one;
(R)-8-(2,2′-dichloro-3′-(6-methoxy-5-((((5-oxopyrrolidin-2-yl)methyl)amino)methyl)pyridin-2-yl)-[1,1′-biphenyl]-3-yl)-3-(((2,4-dimethoxybenzyl)amino)methyl)-4H-pyrido[1,2-a]pyrimidin-4-one;
(S)-2-(1-(((8-(2,2′-dichloro-3′-(6-methoxy-5-((((5-oxopyrrolidin-2-yl)methyl)amino)methyl)pyridin-2-yl)-[1,1′-biphenyl]-3-yl)-4-oxo-4H-pyrido[1,2-a]pyrimidin-3-yl)methyl)amino)cyclopropyl)acetic acid;
(R)-2-(1-(((8-(2,2′-dichloro-3′-(6-methoxy-5-((((5-oxopyrrolidin-2-yl)methyl)amino)methyl)pyridin-2-yl)-[1,1′-biphenyl]-3-yl)-4-oxo-4H-pyrido[1,2-a]pyrimidin-3-yl)methyl)amino)cyclopropyl)acetic acid;
8-(2,2′-dichloro-5-fluoro-3′-(6-methoxy-5-(((((S)-5-oxopyrrolidin-2-yl)methyl)amino)methyl)pyridin-2-yl)-[1,1′-biphenyl]-3-yl)-3-(((((S)-5-oxopyrrolidin-2-yl)methyl)amino)methyl)-4H-pyrido[1,2-a]pyrimidin-4-one;
8-(2,2′-dichloro-5-fluoro-3′-(6-methoxy-5-(((((R)-5-oxopyrrolidin-2-yl)methyl)amino)methyl)pyridin-2-yl)-[1,1′-biphenyl]-3-yl)-3-(((((S)-5-oxopyrrolidin-2-yl)methyl)amino)methyl)-4H-pyrido[1,2-a]pyrimidin-4-one;
8-(2,2′-dichloro-5-fluoro-3′-(6-methoxy-5-(((((S)-5-oxopyrrolidin-2-yl)methyl)amino)methyl)pyridin-2-yl)-[1,1′-biphenyl]-3-yl)-3-(((((R)-5-oxopyrrolidin-2-yl)methyl)amino)methyl)-4H-pyrido[1,2-a]pyrimidin-4-one;
8-(2,2′-dichloro-5-fluoro-3′-(6-methoxy-5-(((((R)-5-oxopyrrolidin-2-yl)methyl)amino)methyl)pyridin-2-yl)-[1,1′-biphenyl]-3-yl)-3-(((((R)-5-oxopyrrolidin-2-yl)methyl)amino)methyl)-4H-pyrido[1,2-a]pyrimidin-4-one;
3-(((((S)-5-oxopyrrolidin-2-yl)methyl)amino)methyl)-8-(2,2′,5-trichloro-3′-(6-methoxy-5-(((((S)-5-oxopyrrolidin-2-yl)methyl)amino)methyl)pyridin-2-yl)-[1,1′-biphenyl]-3-yl)-4H-pyrido[1,2-a]pyrimidin-4-one;
3-(((((R)-5-oxopyrrolidin-2-yl)methyl)amino)methyl)-8-(2,2′,5-trichloro-3′-(6-methoxy-5-(((((S)-5-oxopyrrolidin-2-yl)methyl)amino)methyl)pyridin-2-yl)-[1,1′-biphenyl]-3-yl)-4H-pyrido[1,2-a]pyrimidin-4-one;
3-(((((S)-5-oxopyrrolidin-2-yl)methyl)amino)methyl)-8-(2,2′,5-trichloro-3′-(6-methoxy-5-(((((R)-5-oxopyrrolidin-2-yl)methyl)amino)methyl)pyridin-2-yl)-[1,1′-biphenyl]-3-yl)-4H-pyrido[1,2-a]pyrimidin-4-one;
3-(((((R)-5-oxopyrrolidin-2-yl)methyl)amino)methyl)-8-(2,2′,5-trichloro-3′-(6-methoxy-5-(((((R)-5-oxopyrrolidin-2-yl)methyl)amino)methyl)pyridin-2-yl)-[1,1′-biphenyl]-3-yl)-4H-pyrido[1,2-a]pyrimidin-4-one;
8-(2,2′-dichloro-3′-(5-((((1r,3r)-3-hydroxy-3-methylcyclobutyl)amino)methyl)-6-methoxypyridin-2-yl)-[1,1′-biphenyl]-3-yl)-3-(((((S)-5-oxopyrrolidin-2-yl)methyl)amino)methyl)-4H-pyrido[1,2-a]pyrimidin-4-one;
8-(2,2′-dichloro-3′-(5-((((1r,3r)-3-hydroxy-3-methylcyclobutyl)amino)methyl)-6-methoxypyridin-2-yl)-[1,1′-biphenyl]-3-yl)-3-(((((R)-5-oxopyrrolidin-2-yl)methyl)amino)methyl)-4H-pyrido[1,2-a]pyrimidin-4-one;
8-(2,2′-dichloro-3′-(5-((((1s,3s)-3-hydroxy-3-methylcyclobutyl)amino)methyl)-6-methoxypyridin-2-yl)-[1,1′-biphenyl]-3-yl)-3-(((((S)-5-oxopyrrolidin-2-yl)methyl)amino)methyl)-4H-pyrido[1,2-a]pyrimidin-4-one;
8-(2,2′-dichloro-3′-(5-((((1s,3s)-3-hydroxy-3-methylcyclobutyl)amino)methyl)-6-methoxypyridin-2-yl)-[1,1′-biphenyl]-3-yl)-3-(((((R)-5-oxopyrrolidin-2-yl)methyl)amino)methyl)-4H-pyrido[1,2-a]pyrimidin-4-one;
8-(2,2′-dichloro-3′-(6-methoxy-5-(((((S)-5-oxopyrrolidin-2-yl)methyl)amino)methyl)pyridin-2-yl)-[1,1′-biphenyl]-3-yl)-3-((((1r,3r)-3-hydroxy-3-methylcyclobutyl)amino)methyl)-4H-pyrido[1,2-a]pyrimidin-4-one;
8-(2,2′-dichloro-3′-(6-methoxy-5-(((((R)-5-oxopyrrolidin-2-yl)methyl)amino)methyl)pyridin-2-yl)-[1,1′-biphenyl]-3-yl)-3-((((1s,3s)-3-hydroxy-3-methylcyclobutyl)amino)methyl)-4H-pyrido[1,2-a]pyrimidin-4-one;
8-(2,2′-dichloro-3′-(6-methoxy-5-(((((S)-5-oxopyrrolidin-2-yl)methyl)amino)methyl)pyridin-2-yl)-[1,1′-biphenyl]-3-yl)-3-((((1s,3s)-3-hydroxy-3-methylcyclobutyl)amino)methyl)-4H-pyrido[1,2-a]pyrimidin-4-one;
8-(2,2′-dichloro-3′-(6-methoxy-5-(((((R)-5-oxopyrrolidin-2-yl)methyl)amino)methyl)pyridin-2-yl)-[1,1′-biphenyl]-3-yl)-3-((((1r,3r)-3-hydroxy-3-methylcyclobutyl)amino)methyl)-4H-pyrido[1,2-a]pyrimidin-4-one;
8-(2,2′-dichloro-3′-(6-methoxy-5-(((((S)-5-oxopyrrolidin-2-yl)methyl)amino)methyl)pyridin-2-yl)-[1,1′-biphenyl]-3-yl)-3-(((S)-3-hydroxypyrrolidin-1-yl)methyl)-4H-pyrido[1,2-a]pyrimidin-4-one;
8-(2,2′-dichloro-3′-(6-methoxy-5-(((((R)-5-oxopyrrolidin-2-yl)methyl)amino)methyl)pyridin-2-yl)-[1,1′-biphenyl]-3-yl)-3-(((S)-3-hydroxypyrrolidin-1-yl)methyl)-4H-pyrido[1,2-a]pyrimidin-4-one;
8-(2,2′-dichloro-3′-(6-methoxy-5-(((((S)-5-oxopyrrolidin-2-yl)methyl)amino)methyl)pyridin-2-yl)-[1,1′-biphenyl]-3-yl)-3-(((R)-3-hydroxypyrrolidin-1-yl)methyl)-4H-pyrido[1,2-a]pyrimidin-4-one;
8-(2,2′-dichloro-3′-(6-methoxy-5-(((((R)-5-oxopyrrolidin-2-yl)methyl)amino)methyl)pyridin-2-yl)-[1,1′-biphenyl]-3-yl)-3-(((R)-3-hydroxypyrrolidin-1-yl)methyl)-4H-pyrido[1,2-a]pyrimidin-4-one;
2′-chloro-3-(6-methoxy-5-(((((S)-5-oxopyrrolidin-2-yl)methyl)amino)methyl)pyridin-2-yl)-3′-(4-oxo-3-(((((S)-5-oxopyrrolidin-2-yl)methyl)amino)methyl)-4H-pyrido[1,2-a]pyrimidin-8-yl)-[1,1′-biphenyl]-2-carbonitrile;
2′-chloro-3-(6-methoxy-5-(((((R)-5-oxopyrrolidin-2-yl)methyl)amino)methyl)pyridin-2-yl)-3′-(4-oxo-3-(((((S)-5-oxopyrrolidin-2-yl)methyl)amino)methyl)-4H-pyrido[1,2-a]pyrimidin-8-yl)-[1,1′-biphenyl]-2-carbonitrile;
2′-chloro-3-(6-methoxy-5-(((((S)-5-oxopyrrolidin-2-yl)methyl)amino)methyl)pyridin-2-yl)-3′-(4-oxo-3-(((((R)-5-oxopyrrolidin-2-yl)methyl)amino)methyl)-4H-pyrido[1,2-a]pyrimidin-8-yl)-[1,1′-biphenyl]-2-carbonitrile;
2′-chloro-3-(6-methoxy-5-(((((R)-5-oxopyrrolidin-2-yl)methyl)amino)methyl)pyridin-2-yl)-3′-(4-oxo-3-(((((R)-5-oxopyrrolidin-2-yl)methyl)amino)methyl)-4H-pyrido[1,2-a]pyrimidin-8-yl)-[1,1′-biphenyl]-2-carbonitrile;
8-(2,2′-dichloro-5′-methoxy-3′-(6-methoxy-5-(((((S)-5-oxopyrrolidin-2-yl)methyl)amino)methyl)pyridin-2-yl)-[1,1′-biphenyl]-3-yl)-3-(((((S)-5-oxopyrrolidin-2-yl)methyl)amino)methyl)-4H-pyrido[1,2-a]pyrimidin-4-one;
8-(2,2′-dichloro-5′-methoxy-3′-(6-methoxy-5-(((((R)-5-oxopyrrolidin-2-yl)methyl)amino)methyl)pyridin-2-yl)-[1,1′-biphenyl]-3-yl)-3-(((((S)-5-oxopyrrolidin-2-yl)methyl)amino)methyl)-4H-pyrido[1,2-a]pyrimidin-4-one;
8-(2,2′-dichloro-5′-methoxy-3′-(6-methoxy-5-(((((S)-5-oxopyrrolidin-2-yl)methyl)amino)methyl)pyridin-2-yl)-[1,1′-biphenyl]-3-yl)-3-(((((R)-5-oxopyrrolidin-2-yl)methyl)amino)methyl)-4H-pyrido[1,2-a]pyrimidin-4-one;
8-(2,2′-dichloro-5′-methoxy-3′-(6-methoxy-5-(((((R)-5-oxopyrrolidin-2-yl)methyl)amino)methyl)pyridin-2-yl)-[1,1′-biphenyl]-3-yl)-3-(((((R)-5-oxopyrrolidin-2-yl)methyl)amino)methyl)-4H-pyrido[1,2-a]pyrimidin-4-one;
8-(2,2′-dichloro-3′-(4-fluoro-6-methoxy-5-(((((S)-5-oxopyrrolidin-2-yl)methyl)amino)methyl)pyridin-2-yl)-[1,1′-biphenyl]-3-yl)-3-(((((S)-5-oxopyrrolidin-2-yl)methyl)amino)methyl)-4H-pyrido[1,2-a]pyrimidin-4-one;
8-(2,2′-dichloro-3′-(4-fluoro-6-methoxy-5-(((((R)-5-oxopyrrolidin-2-yl)methyl)amino)methyl)pyridin-2-yl)-[1,1′-biphenyl]-3-yl)-3-(((((S)-5-oxopyrrolidin-2-yl)methyl)amino)methyl)-4H-pyrido[1,2-a]pyrimidin-4-one;
8-(2,2′-dichloro-3′-(4-fluoro-6-methoxy-5-(((((S)-5-oxopyrrolidin-2-yl)methyl)amino)methyl)pyridin-2-yl)-[1,1′-biphenyl]-3-yl)-3-(((((R)-5-oxopyrrolidin-2-yl)methyl)amino)methyl)-4H-pyrido[1,2-a]pyrimidin-4-one;
8-(2,2′-dichloro-3′-(4-fluoro-6-methoxy-5-(((((R)-5-oxopyrrolidin-2-yl)methyl)amino)methyl)pyridin-2-yl)-[1,1′-biphenyl]-3-yl)-3-(((((R)-5-oxopyrrolidin-2-yl)methyl)amino)methyl)-4H-pyrido[1,2-a]pyrimidin-4-one;
(2S,4S)-1-((8-(2,2′-dichloro-3′-(6-methoxy-5-(((((S)-5-oxopyrrolidin-2-yl)methyl)amino)methyl)pyridin-2-yl)-[1,1′-biphenyl]-3-yl)-4-oxo-4H-pyrido[1,2-a]pyrimidin-3-yl)methyl)-4-hydroxypyrrolidine-2-carboxylic acid;
(2R,4S)-1-((8-(2,2′-dichloro-3′-(6-methoxy-5-(((((S)-5-oxopyrrolidin-2-yl)methyl)amino)methyl)pyridin-2-yl)-[1,1′-biphenyl]-3-yl)-4-oxo-4H-pyrido[1,2-a]pyrimidin-3-yl)methyl)-4-hydroxypyrrolidine-2-carboxylic acid;
(2S,4R)-1-((8-(2,2′-dichloro-3′-(6-methoxy-5-(((((S)-5-oxopyrrolidin-2-yl)methyl)amino)methyl)pyridin-2-yl)-[1,1′-biphenyl]-3-yl)-4-oxo-4H-pyrido[1,2-a]pyrimidin-3-yl)methyl)-4-hydroxypyrrolidine-2-carboxylic acid;
(2R,4R)-1-((8-(2,2′-dichloro-3′-(6-methoxy-5-(((((S)-5-oxopyrrolidin-2-yl)methyl)amino)methyl)pyridin-2-yl)-[1,1′-biphenyl]-3-yl)-4-oxo-4H-pyrido[1,2-a]pyrimidin-3-yl)methyl)-4-hydroxypyrrolidine-2-carboxylic acid;
(2S,4S)-1-((8-(2,2′-dichloro-3′-(6-methoxy-5-(((((R)-5-oxopyrrolidin-2-yl)methyl)amino)methyl)pyridin-2-yl)-[1,1′-biphenyl]-3-yl)-4-oxo-4H-pyrido[1,2-a]pyrimidin-3-yl)methyl)-4-hydroxypyrrolidine-2-carboxylic acid;
(2R,4S)-1-((8-(2,2′-dichloro-3′-(6-methoxy-5-(((((R)-5-oxopyrrolidin-2-yl)methyl)amino)methyl)pyridin-2-yl)-[1,1′-biphenyl]-3-yl)-4-oxo-4H-pyrido[1,2-a]pyrimidin-3-yl)methyl)-4-hydroxypyrrolidine-2-carboxylic acid;
(2S,4R)-1-((8-(2,2′-dichloro-3′-(6-methoxy-5-(((((R)-5-oxopyrrolidin-2-yl)methyl)amino)methyl)pyridin-2-yl)-[1,1′-biphenyl]-3-yl)-4-oxo-4H-pyrido[1,2-a]pyrimidin-3-yl)methyl)-4-hydroxypyrrolidine-2-carboxylic acid;
(2R,4R)-1-((8-(2,2′-dichloro-3′-(6-methoxy-5-(((((R)-5-oxopyrrolidin-2-yl)methyl)amino)methyl)pyridin-2-yl)-[1,1′-biphenyl]-3-yl)-4-oxo-4H-pyrido[1,2-a]pyrimidin-3-yl)methyl)-4-hydroxypyrrolidine-2-carboxylic acid;
(2R,4S)-1-((8-(2,2′-dichloro-3′-(6-methoxy-5-(((((S)-5-oxopyrrolidin-2-yl)methyl)amino)methyl)pyridin-2-yl)-[1,1′-biphenyl]-3-yl)-4-oxo-4H-pyrido[1,2-a]pyrimidin-3-yl)methyl)-4-hydroxypyrrolidine-2-carboxylic acid;
(2S,4S)-1-((8-(2,2′-dichloro-3′-(6-methoxy-5-(((((S)-5-oxopyrrolidin-2-yl)methyl)amino)methyl)pyridin-2-yl)-[1,1′-biphenyl]-3-yl)-4-oxo-4H-pyrido[1,2-a]pyrimidin-3-yl)methyl)-4-hydroxypyrrolidine-2-carboxylic acid;
(2R,4R)-1-((8-(2,2′-dichloro-3′-(6-methoxy-5-(((((S)-5-oxopyrrolidin-2-yl)methyl)amino)methyl)pyridin-2-yl)-[1,1′-biphenyl]-3-yl)-4-oxo-4H-pyrido[1,2-a]pyrimidin-3-yl)methyl)-4-hydroxypyrrolidine-2-carboxylic acid;
(2S,4R)-1-((8-(2,2′-dichloro-3′-(6-methoxy-5-(((((S)-5-oxopyrrolidin-2-yl)methyl)amino)methyl)pyridin-2-yl)-[1,1′-biphenyl]-3-yl)-4-oxo-4H-pyrido[1,2-a]pyrimidin-3-yl)methyl)-4-hydroxypyrrolidine-2-carboxylic acid;
(2R,4S)-1-((8-(2,2′-dichloro-3′-(6-methoxy-5-(((((R)-5-oxopyrrolidin-2-yl)methyl)amino)methyl)pyridin-2-yl)-[1,1′-biphenyl]-3-yl)-4-oxo-4H-pyrido[1,2-a]pyrimidin-3-yl)methyl)-4-hydroxypyrrolidine-2-carboxylic acid;
(2S,4S)-1-((8-(2,2′-dichloro-3′-(6-methoxy-5-(((((R)-5-oxopyrrolidin-2-yl)methyl)amino)methyl)pyridin-2-yl)-[1,1′-biphenyl]-3-yl)-4-oxo-4H-pyrido[1,2-a]pyrimidin-3-yl)methyl)-4-hydroxypyrrolidine-2-carboxylic acid;
(2R,4R)-1-((8-(2,2′-dichloro-3′-(6-methoxy-5-(((((R)-5-oxopyrrolidin-2-yl)methyl)amino)methyl)pyridin-2-yl)-[1,1′-biphenyl]-3-yl)-4-oxo-4H-pyrido[1,2-a]pyrimidin-3-yl)methyl)-4-hydroxypyrrolidine-2-carboxylic acid;
(2S,4R)-1-((8-(2,2′-dichloro-3′-(6-methoxy-5-(((((R)-5-oxopyrrolidin-2-yl)methyl)amino)methyl)pyridin-2-yl)-[1,1′-biphenyl]-3-yl)-4-oxo-4H-pyrido[1,2-a]pyrimidin-3-yl)methyl)-4-hydroxypyrrolidine-2-carboxylic acid;
N-((8-(2,2′-dichloro-3′-(6-methoxy-5-(((((S)-5-oxopyrrolidin-2-yl)methyl)amino)methyl)pyridin-2-yl)-[1,1′-biphenyl]-3-yl)-4-oxo-4H-pyrido[1,2-a]pyrimidin-3-yl)methyl)-O-methyl-D-serine;
N-((8-(2,2′-dichloro-3′-(6-methoxy-5-(((((R)-5-oxopyrrolidin-2-yl)methyl)amino)methyl)pyridin-2-yl)-[1,1′-biphenyl]-3-yl)-4-oxo-4H-pyrido[1,2-a]pyrimidin-3-yl)methyl)-O-methyl-D-serine;
N-((8-(2,2′-dichloro-3′-(6-methoxy-5-(((((S)-5-oxopyrrolidin-2-yl)methyl)amino)methyl)pyridin-2-yl)-[1,1′-biphenyl]-3-yl)-4-oxo-4H-pyrido[1,2-a]pyrimidin-3-yl)methyl)-O-methyl-L-serine;
N-((8-(2,2′-dichloro-3′-(6-methoxy-5-(((((R)-5-oxopyrrolidin-2-yl)methyl)amino)methyl)pyridin-2-yl)-[1,1′-biphenyl]-3-yl)-4-oxo-4H-pyrido[1,2-a]pyrimidin-3-yl)methyl)-O-methyl-L-serine;
((8-(2,2′-dichloro-3′-(6-methoxy-5-(((((S)-5-oxopyrrolidin-2-yl)methyl)amino)methyl)pyridin-2-yl)-[1,1′-biphenyl]-3-yl)-4-oxo-4H-pyrido[1,2-a]pyrimidin-3-yl)methyl)-L-serine;
((8-(2,2′-dichloro-3′-(6-methoxy-5-(((((R)-5-oxopyrrolidin-2-yl)methyl)amino)methyl)pyridin-2-yl)-[1,1′-biphenyl]-3-yl)-4-oxo-4H-pyrido[1,2-a]pyrimidin-3-yl)methyl)-L-serine;
((8-(2,2′-dichloro-3′-(6-methoxy-5-(((((S)-5-oxopyrrolidin-2-yl)methyl)amino)methyl)pyridin-2-yl)-[1,1′-biphenyl]-3-yl)-4-oxo-4H-pyrido[1,2-a]pyrimidin-3-yl)methyl)-D-serine;
((8-(2,2′-dichloro-3′-(6-methoxy-5-(((((R)-5-oxopyrrolidin-2-yl)methyl)amino)methyl)pyridin-2-yl)-[1,1′-biphenyl]-3-yl)-4-oxo-4H-pyrido[1,2-a]pyrimidin-3-yl)methyl)-D-serine;
8-(2,2′-dichloro-3′-(6-methoxy-5-(((((S)-5-oxopyrrolidin-2-yl)methyl)amino)methyl)pyridin-2-yl)-[1,1′-biphenyl]-3-yl)-3-(((S)-6-oxohexahydropyrrolo[1,2-a]pyrazin-2(1H)-yl)methyl)-4H-pyrido[1,2-a]pyrimidin-4-one;
8-(2,2′-dichloro-3′-(6-methoxy-5-(((((R)-5-oxopyrrolidin-2-yl)methyl)amino)methyl)pyridin-2-yl)-[1,1′-biphenyl]-3-yl)-3-(((S)-6-oxohexahydropyrrolo[1,2-a]pyrazin-2(1H)-yl)methyl)-4H-pyrido[1,2-a]pyrimidin-4-one;
8-(2,2′-dichloro-3′-(6-methoxy-5-(((((S)-5-oxopyrrolidin-2-yl)methyl)amino)methyl)pyridin-2-yl)-[1,1′-biphenyl]-3-yl)-3-(((R)-6-oxohexahydropyrrolo[1,2-a]pyrazin-2(1H)-yl)methyl)-4H-pyrido[1,2-a]pyrimidin-4-one;
8-(2,2′-dichloro-3′-(6-methoxy-5-(((((R)-5-oxopyrrolidin-2-yl)methyl)amino)methyl)pyridin-2-yl)-[1,1′-biphenyl]-3-yl)-3-(((R)-6-oxohexahydropyrrolo[1,2-a]pyrazin-2(1H)-yl)methyl)-4H-pyrido[1,2-a]pyrimidin-4-one;
8-(2,2′-dichloro-3′-(6-methoxy-5-(((((S)-5-oxopyrrolidin-2-yl)methyl)amino)methyl)pyridin-2-yl)-[1,1′-biphenyl]-3-yl)-3-(((S)-2-(hydroxymethyl)pyrrolidin-1-yl)methyl)-4H-pyrido[1,2-a]pyrimidin-4-one;
8-(2,2′-dichloro-3′-(6-methoxy-5-(((((R)-5-oxopyrrolidin-2-yl)methyl)amino)methyl)pyridin-2-yl)-[1,1′-biphenyl]-3-yl)-3-(((S)-2-(hydroxymethyl)pyrrolidin-1-yl)methyl)-4H-pyrido[1,2-a]pyrimidin-4-one;
8-(2,2′-dichloro-3′-(6-methoxy-5-(((((S)-5-oxopyrrolidin-2-yl)methyl)amino)methyl)pyridin-2-yl)-[1,1′-biphenyl]-3-yl)-3-(((R)-2-(hydroxymethyl)pyrrolidin-1-yl)methyl)-4H-pyrido[1,2-a]pyrimidin-4-one;
8-(2,2′-dichloro-3′-(6-methoxy-5-(((((R)-5-oxopyrrolidin-2-yl)methyl)amino)methyl)pyridin-2-yl)-[1,1′-biphenyl]-3-yl)-3-(((R)-2-(hydroxymethyl)pyrrolidin-1-yl)methyl)-4H-pyrido[1,2-a]pyrimidin-4-one;
(S)-3-(((8-(2,2′-dichloro-3′-(6-methoxy-5-((((5-oxopyrrolidin-2-yl)methyl)amino)methyl)pyridin-2-yl)-[1,1′-biphenyl]-3-yl)-4-oxo-4H-pyrido[1,2-a]pyrimidin-3-yl)methyl)amino)-3-methylbutanoic acid;
(R)-3-(((8-(2,2′-dichloro-3′-(6-methoxy-5-((((5-oxopyrrolidin-2-yl)methyl)amino)methyl)pyridin-2-yl)-[1,1′-biphenyl]-3-yl)-4-oxo-4H-pyrido[1,2-a]pyrimidin-3-yl)methyl)amino)-3-methylbutanoic acid;
(S)-5-((6-(2,2′-dichloro-3′-(6-methoxy-5-(((((S)-5-oxopyrrolidin-2-yl)methyl)amino)methyl)pyridin-2-yl)-[1,1′-biphenyl]-3-yl)-8-methoxy-3,4-dihydroisoquinolin-2(1H)-yl)methyl)pyrrolidin-2-one;
(R)-5-((6-(2,2′-dichloro-3′-(6-methoxy-5-(((((R)-5-oxopyrrolidin-2-yl)methyl)amino)methyl)pyridin-2-yl)-[1,1′-biphenyl]-3-yl)-8-methoxy-3,4-dihydroisoquinolin-2(1H)-yl)methyl)pyrrolidin-2-one;5-((6-(2,2′-dichloro-3′-(R)-(6-methoxy-5-(((((S)-5-oxopyrrolidin-2-yl)methyl)amino)methyl)pyridin-2-yl)-[1,1′-biphenyl]-3-yl)-8-methoxy-3,4-dihydroisoquinolin-2(1H)-yl)methyl)pyrrolidin-2-one;
(S)-5-((6-(2,2′-dichloro-3′-(6-methoxy-5-(((((R)-5-oxopyrrolidin-2-yl)methyl)amino)methyl)pyridin-2-yl)-[1,1′-biphenyl]-3-yl)-8-methoxy-3,4-dihydroisoquinolin-2(1H)-yl)methyl)pyrrolidin-2-one;
8-(2,2′-dichloro-3′-(6-methoxy-5-(((((S)-5-oxopyrrolidin-2-yl)methyl)amino)methyl)pyridin-2-yl)-[1,1′-biphenyl]-3-yl)-2-(((((S)-5-oxopyrrolidin-2-yl)methyl)amino)methyl)-4H-pyrido[1,2-a]pyrimidin-4-one;
8-(2,2′-dichloro-3′-(6-methoxy-5-(((((R)-5-oxopyrrolidin-2-yl)methyl)amino)methyl)pyridin-2-yl)-[1,1′-biphenyl]-3-yl)-2-(((((S)-5-oxopyrrolidin-2-yl)methyl)amino)methyl)-4H-pyrido[1,2-a]pyrimidin-4-one;
8-(2,2′-dichloro-3′-(6-methoxy-5-(((((S)-5-oxopyrrolidin-2-yl)methyl)amino)methyl)pyridin-2-yl)-[1,1′-biphenyl]-3-yl)-2-(((((R)-5-oxopyrrolidin-2-yl)methyl)amino)methyl)-4H-pyrido[1,2-a]pyrimidin-4-one;
8-(2,2′-dichloro-3′-(6-methoxy-5-(((((R)-5-oxopyrrolidin-2-yl)methyl)amino)methyl)pyridin-2-yl)-[1,1′-biphenyl]-3-yl)-2-(((((R)-5-oxopyrrolidin-2-yl)methyl)amino)methyl)-4H-pyrido[1,2-a]pyrimidin-4-one;
(S)-8-(2,2′-dichloro-3′-(5-((3-hydroxy-3-methylazetidin-1-yl)methyl)-6-methoxypyridin-2-yl)-[1,1′-biphenyl]-3-yl)-3-((((5-oxopyrrolidin-2-yl)methyl)amino)methyl)-4H-pyrido[1,2-a]pyrimidin-4-one;
(R)-8-(2,2′-dichloro-3′-(5-((3-hydroxy-3-methylazetidin-1-yl)methyl)-6-methoxypyridin-2-yl)-[1,1′-biphenyl]-3-yl)-3-((((5-oxopyrrolidin-2-yl)methyl)amino)methyl)-4H-pyrido[1,2-a]pyrimidin-4-one;
8-(2,2′-dichloro-3′-(5-((((1r,3r)-3-hydroxycyclobutyl)amino)methyl)-6-methoxypyridin-2-yl)-[1,1′-biphenyl]-3-yl)-3-(((((S)-5-oxopyrrolidin-2-yl)methyl)amino)methyl)-4H-pyrido[1,2-a]pyrimidin-4-one;
8-(2,2′-dichloro-3′-(5-((((1s,3s)-3-hydroxycyclobutyl)amino)methyl)-6-methoxypyridin-2-yl)-[1,1′-biphenyl]-3-yl)-3-(((((S)-5-oxopyrrolidin-2-yl)methyl)amino)methyl)-4H-pyrido[1,2-a]pyrimidin-4-one;
8-(2,2′-dichloro-3′-(5-((((1r,3r)-3-hydroxycyclobutyl)amino)methyl)-6-methoxypyridin-2-yl)-[1,1′-biphenyl]-3-yl)-3-(((((R)-5-oxopyrrolidin-2-yl)methyl)amino)methyl)-4H-pyrido[1,2-a]pyrimidin-4-one;
8-(2,2′-dichloro-3′-(5-((((1s,3s)-3-hydroxycyclobutyl)amino)methyl)-6-methoxypyridin-2-yl)-[1,1′-biphenyl]-3-yl)-3-(((((R)-5-oxopyrrolidin-2-yl)methyl)amino)methyl)-4H-pyrido[1,2-a]pyrimidin-4-one;
(S)-8-(3′-(5-((3,3-bis(hydroxymethyl)azetidin-1-yl)methyl)-6-methoxypyridin-2-yl)-2,2′-dichloro-[1,1′-biphenyl]-3-yl)-3-((((5-oxopyrrolidin-2-yl)methyl)amino)methyl)-4H-pyrido[1,2-a]pyrimidin-4-one;
(R)-8-(3′-(5-((3,3-bis(hydroxymethyl)azetidin-1-yl)methyl)-6-methoxypyridin-2-yl)-2,2′-dichloro-[1,1′-biphenyl]-3-yl)-3-((((5-oxopyrrolidin-2-yl)methyl)amino)methyl)-4H-pyrido[1,2-a]pyrimidin-4-one;
(S)-4-(((8-(2,2′-dichloro-3′-(6-methoxy-5-((((5-oxopyrrolidin-2-yl)methyl)amino)methyl)pyridin-2-yl)-[1,1′-biphenyl]-3-yl)-4-oxo-4H-pyrido[1,2-a]pyrimidin-3-yl)methyl)amino)butanoic acid;
(R)-4-(((8-(2,2′-dichloro-3′-(6-methoxy-5-((((5-oxopyrrolidin-2-yl)methyl)amino)methyl)pyridin-2-yl)-[1,1′-biphenyl]-3-yl)-4-oxo-4H-pyrido[1,2-a]pyrimidin-3-yl)methyl)amino)butanoic acid;
8-(2,2′-dichloro-3′-(6-methoxy-5-(((((S)-5-oxopyrrolidin-2-yl)methyl)amino)methyl)pyridin-2-yl)-[1,1′-biphenyl]-3-yl)-3-(5-hydroxy-1,4,5,6-tetrahydropyrimidin-2-yl)-4H-pyrido[1,2-a]pyrimidin-4-one;
8-(2,2′-dichloro-3′-(6-methoxy-5-(((((R)-5-oxopyrrolidin-2-yl)methyl)amino)methyl)pyridin-2-yl)-[1,1′-biphenyl]-3-yl)-3-(5-hydroxy-1,4,5,6-tetrahydropyrimidin-2-yl)-4H-pyrido[1,2-a]pyrimidin-4-one;
methyl (S)-1-((8-(2,2′-dichloro-3′-(6-methoxy-5-((((5-oxopyrrolidin-2-yl)methyl)amino)methyl)pyridin-2-yl)-[1,1′-biphenyl]-3-yl)-4-oxo-4H-pyrido[1,2-a]pyrimidin-3-yl)methyl)azetidine-3-carboxylate;
methyl (R)-1-((8-(2,2′-dichloro-3′-(6-methoxy-5-((((5-oxopyrrolidin-2-yl)methyl)amino)methyl)pyridin-2-yl)-[1,1′-biphenyl]-3-yl)-4-oxo-4H-pyrido[1,2-a]pyrimidin-3-yl)methyl)azetidine-3-carboxylate;
8-(2,2′-dichloro-3′-(6-methoxy-5-(((((S)-5-oxopyrrolidin-2-yl)methyl)amino)methyl)pyridin-2-yl)-[1,1′-biphenyl]-3-yl)-3-((((3R,4S)-3-hydroxytetrahydro-2H-pyran-4-yl)amino)methyl)-4H-pyrido[1,2-a]pyrimidin-4-one;
8-(2,2′-dichloro-3′-(6-methoxy-5-(((((S)-5-oxopyrrolidin-2-yl)methyl)amino)methyl)pyridin-2-yl)-[1,1′-biphenyl]-3-yl)-3-((((3S,4S)-3-hydroxytetrahydro-2H-pyran-4-yl)amino)methyl)-4H-pyrido[1,2-a]pyrimidin-4-one;
8-(2,2′-dichloro-3′-(6-methoxy-5-(((((S)-5-oxopyrrolidin-2-yl)methyl)amino)methyl)pyridin-2-yl)-[1,1′-biphenyl]-3-yl)-3-((((3R,4R)-3-hydroxytetrahydro-2H-pyran-4-yl)amino)methyl)-4H-pyrido[1,2-a]pyrimidin-4-one;
8-(2,2′-dichloro-3′-(6-methoxy-5-(((((S)-5-oxopyrrolidin-2-yl)methyl)amino)methyl)pyridin-2-yl)-[1,1′-biphenyl]-3-yl)-3-((((3S,4R)-3-hydroxytetrahydro-2H-pyran-4-yl)amino)methyl)-4H-pyrido[1,2-a]pyrimidin-4-one;
8-(2,2′-dichloro-3′-(6-methoxy-5-(((((R)-5-oxopyrrolidin-2-yl)methyl)amino)methyl)pyridin-2-yl)-[1,1′-biphenyl]-3-yl)-3-((((3R,4S)-3-hydroxytetrahydro-2H-pyran-4-yl)amino)methyl)-4H-pyrido[1,2-a]pyrimidin-4-one;
8-(2,2′-dichloro-3′-(6-methoxy-5-(((((R)-5-oxopyrrolidin-2-yl)methyl)amino)methyl)pyridin-2-yl)-[1,1′-biphenyl]-3-yl)-3-((((3S,4S)-3-hydroxytetrahydro-2H-pyran-4-yl)amino)methyl)-4H-pyrido[1,2-a]pyrimidin-4-one;
8-(2,2′-dichloro-3′-(6-methoxy-5-(((((R)-5-oxopyrrolidin-2-yl)methyl)amino)methyl)pyridin-2-yl)-[1,1′-biphenyl]-3-yl)-3-((((3R,4R)-3-hydroxytetrahydro-2H-pyran-4-yl)amino)methyl)-4H-pyrido[1,2-a]pyrimidin-4-one;
8-(2,2′-dichloro-3′-(6-methoxy-5-(((((R)-5-oxopyrrolidin-2-yl)methyl)amino)methyl)pyridin-2-yl)-[1,1′-biphenyl]-3-yl)-3-((((3S,4R)-3-hydroxytetrahydro-2H-pyran-4-yl)amino)methyl)-4H-pyrido[1,2-a]pyrimidin-4-one;
8-(2,2′-dichloro-3′-(6-methoxy-5-(((((S)-5-oxopyrrolidin-2-yl)methyl)amino)methyl)pyridin-2-yl)-[1,1′-biphenyl]-3-yl)-2-methyl-3-(((((S)-5-oxopyrrolidin-2-yl)methyl)amino)methyl)-4H-pyrido[1,2-a]pyrimidin-4-one;
8-(2,2′-dichloro-3′-(6-methoxy-5-(((((R)-5-oxopyrrolidin-2-yl)methyl)amino)methyl)pyridin-2-yl)-[1,1′-biphenyl]-3-yl)-2-methyl-3-(((((S)-5-oxopyrrolidin-2-yl)methyl)amino)methyl)-4H-pyrido[1,2-a]pyrimidin-4-one;
8-(2,2′-dichloro-3′-(6-methoxy-5-(((((S)-5-oxopyrrolidin-2-yl)methyl)amino)methyl)pyridin-2-yl)-[1,1′-biphenyl]-3-yl)-2-methyl-3-(((((R)-5-oxopyrrolidin-2-yl)methyl)amino)methyl)-4H-pyrido[1,2-a]pyrimidin-4-one;
8-(2,2′-dichloro-3′-(6-methoxy-5-(((((R)-5-oxopyrrolidin-2-yl)methyl)amino)methyl)pyridin-2-yl)-[1,1′-biphenyl]-3-yl)-2-methyl-3-(((((R)-5-oxopyrrolidin-2-yl)methyl)amino)methyl)-4H-pyrido[1,2-a]pyrimidin-4-one;
8-(2,2′-dichloro-3′-(5-(((S)-3-hydroxypyrrolidin-1-yl)methyl)-6-methoxypyridin-2-yl)-[1,1′-biphenyl]-3-yl)-3-(((S)-3-hydroxypyrrolidin-1-yl)methyl)-4H-pyrido[1,2-a]pyrimidin-4-one;
8-(2,2′-dichloro-3′-(5-(((R)-3-hydroxypyrrolidin-1-yl)methyl)-6-methoxypyridin-2-yl)-[1,1′-biphenyl]-3-yl)-3-(((S)-3-hydroxypyrrolidin-1-yl)methyl)-4H-pyrido[1,2-a]pyrimidin-4-one;
8-(2,2′-dichloro-3′-(5-(((S)-3-hydroxypyrrolidin-1-yl)methyl)-6-methoxypyridin-2-yl)-[1,1′-biphenyl]-3-yl)-3-(((R)-3-hydroxypyrrolidin-1-yl)methyl)-4H-pyrido[1,2-a]pyrimidin-4-one;
8-(2,2′-dichloro-3′-(5-(((R)-3-hydroxypyrrolidin-1-yl)methyl)-6-methoxypyridin-2-yl)-[1,1′-biphenyl]-3-yl)-3-(((R)-3-hydroxypyrrolidin-1-yl)methyl)-4H-pyrido[1,2-a]pyrimidin-4-one;
(2R,4R)-1-((8-(2,2′-dichloro-3′-(6-methoxy-5-(((((S)-5-oxopyrrolidin-2-yl)methyl)amino)methyl)pyridin-2-yl)-[1,1′-biphenyl]-3-yl)-4-oxo-4H-pyrido[1,2-a]pyrimidin-3-yl)methyl)-4-hydroxypyrrolidine-2-carboxylic acid;
(2S,4R)-1-((8-(2,2′-dichloro-3′-(6-methoxy-5-(((((S)-5-oxopyrrolidin-2-yl)methyl)amino)methyl)pyridin-2-yl)-[1,1′-biphenyl]-3-yl)-4-oxo-4H-pyrido[1,2-a]pyrimidin-3-yl)methyl)-4-hydroxypyrrolidine-2-carboxylic acid;
(2R,4S)-1-((8-(2,2′-dichloro-3′-(6-methoxy-5-(((((S)-5-oxopyrrolidin-2-yl)methyl)amino)methyl)pyridin-2-yl)-[1,1′-biphenyl]-3-yl)-4-oxo-4H-pyrido[1,2-a]pyrimidin-3-yl)methyl)-4-hydroxypyrrolidine-2-carboxylic acid;
(2S,4SR)-1-((8-(2,2′-dichloro-3′-(6-methoxy-5-(((((S)-5-oxopyrrolidin-2-yl)methyl)amino)methyl)pyridin-2-yl)-[1,1′-biphenyl]-3-yl)-4-oxo-4H-pyrido[1,2-a]pyrimidin-3-yl)methyl)-4-hydroxypyrrolidine-2-carboxylic acid;
(2R,4R)-1-((8-(2,2′-dichloro-3′-(6-methoxy-5-(((((R)-5-oxopyrrolidin-2-yl)methyl)amino)methyl)pyridin-2-yl)-[1,1′-biphenyl]-3-yl)-4-oxo-4H-pyrido[1,2-a]pyrimidin-3-yl)methyl)-4-hydroxypyrrolidine-2-carboxylic acid;
(2S,4R)-1-((8-(2,2′-dichloro-3′-(6-methoxy-5-(((((R)-5-oxopyrrolidin-2-yl)methyl)amino)methyl)pyridin-2-yl)-[1,1′-biphenyl]-3-yl)-4-oxo-4H-pyrido[1,2-a]pyrimidin-3-yl)methyl)-4-hydroxypyrrolidine-2-carboxylic acid;
(2R,4S)-1-((8-(2,2′-dichloro-3′-(6-methoxy-5-(((((R)-5-oxopyrrolidin-2-yl)methyl)amino)methyl)pyridin-2-yl)-[1,1′-biphenyl]-3-yl)-4-oxo-4H-pyrido[1,2-a]pyrimidin-3-yl)methyl)-4-hydroxypyrrolidine-2-carboxylic acid;
(2S,4SR)-1-((8-(2,2′-dichloro-3′-(6-methoxy-5-(((((R)-5-oxopyrrolidin-2-yl)methyl)amino)methyl)pyridin-2-yl)-[1,1′-biphenyl]-3-yl)-4-oxo-4H-pyrido[1,2-a]pyrimidin-3-yl)methyl)-4-hydroxypyrrolidine-2-carboxylic acid;
methyl (S)-2-(8-(2,2′-dichloro-3′-(6-methoxy-5-((((5-oxopyrrolidin-2-yl)methyl)amino)methyl)pyridin-2-yl)-[1,1′-biphenyl]-3-yl)-4-oxo-4H-pyrido[1,2-a]pyrimidin-3-yl)-1H-imidazole-5-carboxylate;
methyl (R)-2-(8-(2,2′-dichloro-3′-(6-methoxy-5-((((5-oxopyrrolidin-2-yl)methyl)amino)methyl)pyridin-2-yl)-[1,1′-biphenyl]-3-yl)-4-oxo-4H-pyrido[1,2-a]pyrimidin-3-yl)-1H-imidazole-5-carboxylate;
(S)-1-((8-(2,2′-dichloro-3′-(5-(((2-hydroxyethyl)amino)methyl)-6-methoxypyridin-2-yl)-[1,1′-biphenyl]-3-yl)-4-oxo-4H-pyrido[1,2-a]pyrimidin-3-yl)methyl)-2-methylpyrrolidine-2-carboxylic acid;
(R)-1-((8-(2,2′-dichloro-3′-(5-(((2-hydroxyethyl)amino)methyl)-6-methoxypyridin-2-yl)-[1,1′-biphenyl]-3-yl)-4-oxo-4H-pyrido[1,2-a]pyrimidin-3-yl)methyl)-2-methylpyrrolidine-2-carboxylic acid;
8-(2,2′-dichloro-3′-(6-methoxy-5-(((((S)-5-oxopyrrolidin-2-yl)methyl)amino)methyl)pyridin-2-yl)-[1,1′-biphenyl]-3-yl)-3-(((R)-3-hydroxypyrrolidin-1-yl)methyl)-4H-pyrido[1,2-a]pyrimidin-4-one;
8-(2,2′-dichloro-3′-(6-methoxy-5-(((((R)-5-oxopyrrolidin-2-yl)methyl)amino)methyl)pyridin-2-yl)-[1,1′-biphenyl]-3-yl)-3-(((R)-3-hydroxypyrrolidin-1-yl)methyl)-4H-pyrido[1,2-a]pyrimidin-4-one;
8-(2,2′-dichloro-3′-(6-methoxy-5-(((((S)-5-oxopyrrolidin-2-yl)methyl)amino)methyl)pyridin-2-yl)-[1,1′-biphenyl]-3-yl)-3-(((S)-3-hydroxypyrrolidin-1-yl)methyl)-4H-pyrido[1,2-a]pyrimidin-4-one;
8-(2,2′-dichloro-3′-(6-methoxy-5-(((((R)-5-oxopyrrolidin-2-yl)methyl)amino)methyl)pyridin-2-yl)-[1,1′-biphenyl]-3-yl)-3-(((S)-3-hydroxypyrrolidin-1-yl)methyl)-4H-pyrido[1,2-a]pyrimidin-4-one;
8-(2,2′-dichloro-3′-(5-(((R)-3-hydroxypyrrolidin-1-yl)methyl)-6-methoxypyridin-2-yl)-[1,1′-biphenyl]-3-yl)-3-(((((S)-5-oxopyrrolidin-2-yl)methyl)amino)methyl)-4H-pyrido[1,2-a]pyrimidin-4-one;
8-(2,2′-dichloro-3′-(5-(((S)-3-hydroxypyrrolidin-1-yl)methyl)-6-methoxypyridin-2-yl)-[1,1′-biphenyl]-3-yl)-3-(((((S)-5-oxopyrrolidin-2-yl)methyl)amino)methyl)-4H-pyrido[1,2-a]pyrimidin-4-one;
8-(2,2′-dichloro-3′-(5-(((R)-3-hydroxypyrrolidin-1-yl)methyl)-6-methoxypyridin-2-yl)-[1,1′-biphenyl]-3-yl)-3-(((((R)-5-oxopyrrolidin-2-yl)methyl)amino)methyl)-4H-pyrido[1,2-a]pyrimidin-4-one;
8-(2,2′-dichloro-3′-(5-(((S)-3-hydroxypyrrolidin-1-yl)methyl)-6-methoxypyridin-2-yl)-[1,1′-biphenyl]-3-yl)-3-(((((R)-5-oxopyrrolidin-2-yl)methyl)amino)methyl)-4H-pyrido[1,2-a]pyrimidin-4-one;
methyl (S)-2-(8-(2,2′-dichloro-3′-(6-methoxy-5-(((((S)-5-oxopyrrolidin-2-yl)methyl)amino)methyl)pyridin-2-yl)-[1,1′-biphenyl]-3-yl)-4-oxo-4H-pyrido[1,2-a]pyrimidin-3-yl)-4,5-dihydro-1H-imidazole-5-carboxylate;
methyl (R)-2-(8-(2,2′-dichloro-3′-(6-methoxy-5-(((((S)-5-oxopyrrolidin-2-yl)methyl)amino)methyl)pyridin-2-yl)-[1,1′-biphenyl]-3-yl)-4-oxo-4H-pyrido[1,2-a]pyrimidin-3-yl)-4,5-dihydro-1H-imidazole-5-carboxylate;
methyl (S)-2-(8-(2,2′-dichloro-3′-(6-methoxy-5-(((((R)-5-oxopyrrolidin-2-yl)methyl)amino)methyl)pyridin-2-yl)-[1,1′-biphenyl]-3-yl)-4-oxo-4H-pyrido[1,2-a]pyrimidin-3-yl)-4,5-dihydro-1H-imidazole-5-carboxylate;
methyl (R)-2-(8-(2,2′-dichloro-3′-(6-methoxy-5-(((((R)-5-oxopyrrolidin-2-yl)methyl)amino)methyl)pyridin-2-yl)-[1,1′-biphenyl]-3-yl)-4-oxo-4H-pyrido[1,2-a]pyrimidin-3-yl)-4,5-dihydro-1H-imidazole-5-carboxylate;
(S)-1-((8-(2,2′-dichloro-3′-(5-(((2-hydroxyethyl)amino)methyl)-6-methoxypyridin-2-yl)-[1,1′-biphenyl]-3-yl)-4-oxo-4H-pyrido[1,2-a]pyrimidin-3-yl)methyl)pyrrolidine-3-carboxylic acid;
(R)-1-((8-(2,2′-dichloro-3′-(5-(((2-hydroxyethyl)amino)methyl)-6-methoxypyridin-2-yl)-[1,1′-biphenyl]-3-yl)-4-oxo-4H-pyrido[1,2-a]pyrimidin-3-yl)methyl)pyrrolidine-3-carboxylic acid;
(S)-1-(5-chloro-2-(2-cyanoethoxy)-4-((E)-2-(2,2′-dichloro-3′-(4-oxo-3-(((((S)-5-oxopyrrolidin-2-yl)methyl)amino)methyl)-4H-pyrido[1,2-a]pyrimidin-8-yl)-[1,1′-biphenyl]-3-yl)vinyl)benzyl)pyrrolidine-3-carboxylic acid;
(R)-1-(5-chloro-2-(2-cyanoethoxy)-4-((E)-2-(2,2′-dichloro-3′-(4-oxo-3-(((((S)-5-oxopyrrolidin-2-yl)methyl)amino)methyl)-4H-pyrido[1,2-a]pyrimidin-8-yl)-[1,1′-biphenyl]-3-yl)vinyl)benzyl)pyrrolidine-3-carboxylic acid;
(S)-1-(5-chloro-2-(2-cyanoethoxy)-4-((E)-2-(2,2′-dichloro-3′-(4-oxo-3-(((((R)-5-oxopyrrolidin-2-yl)methyl)amino)methyl)-4H-pyrido[1,2-a]pyrimidin-8-yl)-[1,1′-biphenyl]-3-yl)vinyl)benzyl)pyrrolidine-3-carboxylic acid;
(R)-1-(5-chloro-2-(2-cyanoethoxy)-4-((E)-2-(2,2′-dichloro-3′-(4-oxo-3-(((((R)-5-oxopyrrolidin-2-yl)methyl)amino)methyl)-4H-pyrido[1,2-a]pyrimidin-8-yl)-[1,1′-biphenyl]-3-yl)vinyl)benzyl)pyrrolidine-3-carboxylic acid;
(S)-1-(5-chloro-2-(2-cyanoethoxy)-4-((Z)-2-(2,2′-dichloro-3′-(4-oxo-3-(((((S)-5-oxopyrrolidin-2-yl)methyl)amino)methyl)-4H-pyrido[1,2-a]pyrimidin-8-yl)-[1,1′-biphenyl]-3-yl)vinyl)benzyl)pyrrolidine-3-carboxylic acid;
(R)-1-(5-chloro-2-(2-cyanoethoxy)-4-((Z)-2-(2,2′-dichloro-3′-(4-oxo-3-(((((S)-5-oxopyrrolidin-2-yl)methyl)amino)methyl)-4H-pyrido[1,2-a]pyrimidin-8-yl)-[1,1′-biphenyl]-3-yl)vinyl)benzyl)pyrrolidine-3-carboxylic acid;
(S)-1-(5-chloro-2-(2-cyanoethoxy)-4-((Z)-2-(2,2′-dichloro-3′-(4-oxo-3-(((((R)-5-oxopyrrolidin-2-yl)methyl)amino)methyl)-4H-pyrido[1,2-a]pyrimidin-8-yl)-[1,1′-biphenyl]-3-yl)vinyl)benzyl)pyrrolidine-3-carboxylic acid;
(R)-1-(5-chloro-2-(2-cyanoethoxy)-4-((Z)-2-(2,2′-dichloro-3′-(4-oxo-3-(((((R)-5-oxopyrrolidin-2-yl)methyl)amino)methyl)-4H-pyrido[1,2-a]pyrimidin-8-yl)-[1,1′-biphenyl]-3-yl)vinyl)benzyl)pyrrolidine-3-carboxylic acid;
8-(2,2′-dichloro-3′-(5-((((3R,4S)-3-hydroxytetrahydro-2H-pyran-4-yl)amino)methyl)-6-methoxypyridin-2-yl)-[1,1′-biphenyl]-3-yl)-3-(((((S)-5-oxopyrrolidin-2-yl)methyl)amino)methyl)-4H-pyrido[1,2-a]pyrimidin-4-one;
8-(2,2′-dichloro-3′-(5-((((3R,4R)-3-hydroxytetrahydro-2H-pyran-4-yl)amino)methyl)-6-methoxypyridin-2-yl)-[1,1′-biphenyl]-3-yl)-3-(((((S)-5-oxopyrrolidin-2-yl)methyl)amino)methyl)-4H-pyrido[1,2-a]pyrimidin-4-one;
8-(2,2′-dichloro-3′-(5-((((3S,4S)-3-hydroxytetrahydro-2H-pyran-4-yl)amino)methyl)-6-methoxypyridin-2-yl)-[1,1′-biphenyl]-3-yl)-3-(((((S)-5-oxopyrrolidin-2-yl)methyl)amino)methyl)-4H-pyrido[1,2-a]pyrimidin-4-one;
8-(2,2′-dichloro-3′-(5-((((3S,4R)-3-hydroxytetrahydro-2H-pyran-4-yl)amino)methyl)-6-methoxypyridin-2-yl)-[1,1′-biphenyl]-3-yl)-3-(((((S)-5-oxopyrrolidin-2-yl)methyl)amino)methyl)-4H-pyrido[1,2-a]pyrimidin-4-one;
8-(2,2′-dichloro-3′-(5-((((3R,4S)-3-hydroxytetrahydro-2H-pyran-4-yl)amino)methyl)-6-methoxypyridin-2-yl)-[1,1′-biphenyl]-3-yl)-3-(((((R)-5-oxopyrrolidin-2-yl)methyl)amino)methyl)-4H-pyrido[1,2-a]pyrimidin-4-one;
8-(2,2′-dichloro-3′-(5-((((3R,4R)-3-hydroxytetrahydro-2H-pyran-4-yl)amino)methyl)-6-methoxypyridin-2-yl)-[1,1′-biphenyl]-3-yl)-3-(((((R)-5-oxopyrrolidin-2-yl)methyl)amino)methyl)-4H-pyrido[1,2-a]pyrimidin-4-one;
8-(2,2′-dichloro-3′-(5-((((3S,4S)-3-hydroxytetrahydro-2H-pyran-4-yl)amino)methyl)-6-methoxypyridin-2-yl)-[1,1′-biphenyl]-3-yl)-3-(((((R)-5-oxopyrrolidin-2-yl)methyl)amino)methyl)-4H-pyrido[1,2-a]pyrimidin-4-one;
8-(2,2′-dichloro-3′-(5-((((3S,4R)-3-hydroxytetrahydro-2H-pyran-4-yl)amino)methyl)-6-methoxypyridin-2-yl)-[1,1′-biphenyl]-3-yl)-3-(((((R)-5-oxopyrrolidin-2-yl)methyl)amino)methyl)-4H-pyrido[1,2-a]pyrimidin-4-one;
8-(2,2′-dichloro-3′-(5-(((S)-3-hydroxypyrrolidin-1-yl)methyl)-6-methoxypyridin-2-yl)-[1,1′-biphenyl]-3-yl)-3-(((((S)-5-oxopyrrolidin-2-yl)methyl)amino)methyl)-4H-pyrido[1,2-a]pyrimidin-4-one;
8-(2,2′-dichloro-3′-(5-(((R)-3-hydroxypyrrolidin-1-yl)methyl)-6-methoxypyridin-2-yl)-[1,1′-biphenyl]-3-yl)-3-(((((S)-5-oxopyrrolidin-2-yl)methyl)amino)methyl)-4H-pyrido[1,2-a]pyrimidin-4-one;
8-(2,2′-dichloro-3′-(5-(((S)-3-hydroxypyrrolidin-1-yl)methyl)-6-methoxypyridin-2-yl)-[1,1′-biphenyl]-3-yl)-3-(((((R)-5-oxopyrrolidin-2-yl)methyl)amino)methyl)-4H-pyrido[1,2-a]pyrimidin-4-one;
8-(2,2′-dichloro-3′-(5-(((R)-3-hydroxypyrrolidin-1-yl)methyl)-6-methoxypyridin-2-yl)-[1,1′-biphenyl]-3-yl)-3-(((((R)-5-oxopyrrolidin-2-yl)methyl)amino)methyl)-4H-pyrido[1,2-a]pyrimidin-4-one;
8-(2,2′-dichloro-3′-(5-methoxy-6-(((((S)-5-oxopyrrolidin-2-yl)methyl)amino)methyl)quinolin-2-yl)-[1,1′-biphenyl]-3-yl)-3-(((((S)-5-oxopyrrolidin-2-yl)methyl)amino)methyl)-4H-pyrido[1,2-a]pyrimidin-4-one;
8-(2,2′-dichloro-3′-(5-methoxy-6-(((((R)-5-oxopyrrolidin-2-yl)methyl)amino)methyl)quinolin-2-yl)-[1,1′-biphenyl]-3-yl)-3-(((((S)-5-oxopyrrolidin-2-yl)methyl)amino)methyl)-4H-pyrido[1,2-a]pyrimidin-4-one;
8-(2,2′-dichloro-3′-(5-methoxy-6-(((((S)-5-oxopyrrolidin-2-yl)methyl)amino)methyl)quinolin-2-yl)-[1,1′-biphenyl]-3-yl)-3-(((((R)-5-oxopyrrolidin-2-yl)methyl)amino)methyl)-4H-pyrido[1,2-a]pyrimidin-4-one;
8-(2,2′-dichloro-3′-(5-methoxy-6-(((((R)-5-oxopyrrolidin-2-yl)methyl)amino)methyl)quinolin-2-yl)-[1,1′-biphenyl]-3-yl)-3-(((((R)-5-oxopyrrolidin-2-yl)methyl)amino)methyl)-4H-pyrido[1,2-a]pyrimidin-4-one;
8-(2-chloro-3-((E)-1-((3-chloro-6-methoxy-5-(((((S)-5-oxopyrrolidin-2-yl)methyl)amino)methyl)pyridin-2-yl)methylene)-2,3-dihydro-1H-inden-4-yl)phenyl)-3-(((((S)-5-oxopyrrolidin-2-yl)methyl)amino)methyl)-4H-pyrido[1,2-a]pyrimidin-4-one;
8-(2-chloro-3-((E)-1-((3-chloro-6-methoxy-5-(((((R)-5-oxopyrrolidin-2-yl)methyl)amino)methyl)pyridin-2-yl)methylene)-2,3-dihydro-1H-inden-4-yl)phenyl)-3-(((((S)-5-oxopyrrolidin-2-yl)methyl)amino)methyl)-4H-pyrido[1,2-a]pyrimidin-4-one;
8-(2-chloro-3-((E)-1-((3-chloro-6-methoxy-5-(((((S)-5-oxopyrrolidin-2-yl)methyl)amino)methyl)pyridin-2-yl)methylene)-2,3-dihydro-1H-inden-4-yl)phenyl)-3-(((((R)-5-oxopyrrolidin-2-yl)methyl)amino)methyl)-4H-pyrido[1,2-a]pyrimidin-4-one;
8-(2-chloro-3-((E)-1-((3-chloro-6-methoxy-5-(((((R)-5-oxopyrrolidin-2-yl)methyl)amino)methyl)pyridin-2-yl)methylene)-2,3-dihydro-1H-inden-4-yl)phenyl)-3-(((((R)-5-oxopyrrolidin-2-yl)methyl)amino)methyl)-4H-pyrido[1,2-a]pyrimidin-4-one;
8-(2-chloro-3-((Z)-1-((3-chloro-6-methoxy-5-(((((S)-5-oxopyrrolidin-2-yl)methyl)amino)methyl)pyridin-2-yl)methylene)-2,3-dihydro-1H-inden-4-yl)phenyl)-3-(((((S)-5-oxopyrrolidin-2-yl)methyl)amino)methyl)-4H-pyrido[1,2-a]pyrimidin-4-one;
8-(2-chloro-3-((Z)-1-((3-chloro-6-methoxy-5-(((((R)-5-oxopyrrolidin-2-yl)methyl)amino)methyl)pyridin-2-yl)methylene)-2,3-dihydro-1H-inden-4-yl)phenyl)-3-(((((S)-5-oxopyrrolidin-2-yl)methyl)amino)methyl)-4H-pyrido[1,2-a]pyrimidin-4-one;
8-(2-chloro-3-((Z)-1-((3-chloro-6-methoxy-5-(((((S)-5-oxopyrrolidin-2-yl)methyl)amino)methyl)pyridin-2-yl)methylene)-2,3-dihydro-1H-inden-4-yl)phenyl)-3-(((((R)-5-oxopyrrolidin-2-yl)methyl)amino)methyl)-4H-pyrido[1,2-a]pyrimidin-4-one;
8-(2-chloro-3-((Z)-1-((3-chloro-6-methoxy-5-(((((R)-5-oxopyrrolidin-2-yl)methyl)amino)methyl)pyridin-2-yl)methylene)-2,3-dihydro-1H-inden-4-yl)phenyl)-3-(((((R)-5-oxopyrrolidin-2-yl)methyl)amino)methyl)-4H-pyrido[1,2-a]pyrimidin-4-one;
8-(2,2′-dichloro-3′-(5-(5-hydroxy-1,4,5,6-tetrahydropyrimidin-2-yl)-6-methoxypyridin-2-yl)-[1,1′-biphenyl]-3-yl)-3-(((((S)-5-oxopyrrolidin-2-yl)methyl)amino)methyl)-4H-pyrido[1,2-a]pyrimidin-4-one;
8-(2,2′-dichloro-3′-(5-(5-hydroxy-1,4,5,6-tetrahydropyrimidin-2-yl)-6-methoxypyridin-2-yl)-[1,1′-biphenyl]-3-yl)-3-(((((R)-5-oxopyrrolidin-2-yl)methyl)amino)methyl)-4H-pyrido[1,2-a]pyrimidin-4-one;
2-(8-(2,2′-dichloro-3′-(6-methoxy-5-(((((S)-5-oxopyrrolidin-2-yl)methyl)amino)methyl)pyridin-2-yl)-[1,1′-biphenyl]-3-yl)-4-oxo-4H-pyrido[1,2-a]pyrimidin-3-yl)-4,5-dihydro-1H-imidazole-(5S)-carboxylic acid;
2-(8-(2,2′-dichloro-3′-(6-methoxy-5-(((((R)-5-oxopyrrolidin-2-yl)methyl)amino)methyl)pyridin-2-yl)-[1,1′-biphenyl]-3-yl)-4-oxo-4H-pyrido[1,2-a]pyrimidin-3-yl)-4,5-dihydro-1H-imidazole-(5S)-carboxylic acid;
2-(8-(2,2′-dichloro-3′-(6-methoxy-5-(((((S)-5-oxopyrrolidin-2-yl)methyl)amino)methyl)pyridin-2-yl)-[1,1′-biphenyl]-3-yl)-4-oxo-4H-pyrido[1,2-a]pyrimidin-3-yl)-4,5-dihydro-1H-imidazole-(5R)-carboxylic acid;
2-(8-(2,2′-dichloro-3′-(6-methoxy-5-(((((R)-5-oxopyrrolidin-2-yl)methyl)amino)methyl)pyridin-2-yl)-[1,1′-biphenyl]-3-yl)-4-oxo-4H-pyrido[1,2-a]pyrimidin-3-yl)-4,5-dihydro-1H-imidazole-(5R)-carboxylic acid;
2-(6-(2,2′-dichloro-3′-(4-oxo-3-(((((S)-5-oxopyrrolidin-2-yl)methyl)amino)methyl)-4H-pyrido[1,2-a]pyrimidin-8-yl)-[1,1′-biphenyl]-3-yl)-2-methoxypyridin-3-yl)-3,4,5,6-tetrahydropyrimidine-(4S)-carboxylic acid;
2-(6-(2,2′-dichloro-3′-(4-oxo-3-(((((R)-5-oxopyrrolidin-2-yl)methyl)amino)methyl)-4H-pyrido[1,2-a]pyrimidin-8-yl)-[1,1′-biphenyl]-3-yl)-2-methoxypyridin-3-yl)-3,4,5,6-tetrahydropyrimidine-(4S)-carboxylic acid;
2-(6-(2,2′-dichloro-3′-(4-oxo-3-(((((S)-5-oxopyrrolidin-2-yl)methyl)amino)methyl)-4H-pyrido[1,2-a]pyrimidin-8-yl)-[1,1′-biphenyl]-3-yl)-2-methoxypyridin-3-yl)-3,4,5,6-tetrahydropyrimidine-(4R)-carboxylic acid;
2-(6-(2,2′-dichloro-3′-(4-oxo-3-(((((R)-5-oxopyrrolidin-2-yl)methyl)amino)methyl)-4H-pyrido[1,2-a]pyrimidin-8-yl)-[1,1′-biphenyl]-3-yl)-2-methoxypyridin-3-yl)-3,4,5,6-tetrahydropyrimidine-(4R)-carboxylic acid;
8-(2,2′-dichloro-3′-(5-((((S)-2-hydroxypropyl)amino)methyl)-6-methoxypyridin-2-yl)-[1,1′-biphenyl]-3-yl)-3-((((S)-2-hydroxypropyl)amino)methyl)-2-methyl-4H-pyrido[1,2-a]pyrimidin-4-one;
8-(2,2′-dichloro-3′-(5-((((R)-2-hydroxypropyl)amino)methyl)-6-methoxypyridin-2-yl)-[1,1′-biphenyl]-3-yl)-3-((((S)-2-hydroxypropyl)amino)methyl)-2-methyl-4H-pyrido[1,2-a]pyrimidin-4-one;
8-(2,2′-dichloro-3′-(5-((((S)-2-hydroxypropyl)amino)methyl)-6-methoxypyridin-2-yl)-[1,1′-biphenyl]-3-yl)-3-((((R)-2-hydroxypropyl)amino)methyl)-2-methyl-4H-pyrido[1,2-a]pyrimidin-4-one;
8-(2,2′-dichloro-3′-(5-((((R)-2-hydroxypropyl)amino)methyl)-6-methoxypyridin-2-yl)-[1,1′-biphenyl]-3-yl)-3-((((R)-2-hydroxypropyl)amino)methyl)-2-methyl-4H-pyrido[1,2-a]pyrimidin-4-one;
(S)-3-(((8-(2,2′-dichloro-3′-(6-methoxy-5-((((5-oxopyrrolidin-2-yl)methyl)amino)methyl)pyridin-2-yl)-[1,1′-biphenyl]-3-yl)-4-oxo-4H-pyrido[1,2-a]pyrimidin-3-yl)methyl)amino)propanoic acid;
(R)-3-(((8-(2,2′-dichloro-3′-(6-methoxy-5-((((5-oxopyrrolidin-2-yl)methyl)amino)methyl)pyridin-2-yl)-[1,1′-biphenyl]-3-yl)-4-oxo-4H-pyrido[1,2-a]pyrimidin-3-yl)methyl)amino)propanoic acid;
8-(2,2′-dichloro-3′-(6-methoxy-5-(((((S)-5-oxopyrrolidin-2-yl)methyl)amino)methyl)pyridin-2-yl)-[1,1′-biphenyl]-3-yl)-6-methyl-3-(((((S)-5-oxopyrrolidin-2-yl)methyl)amino)methyl)-4H-pyrido[1,2-a]pyrimidin-4-one;
8-(2,2′-dichloro-3′-(6-methoxy-5-(((((R)-5-oxopyrrolidin-2-yl)methyl)amino)methyl)pyridin-2-yl)-[1,1′-biphenyl]-3-yl)-6-methyl-3-(((((S)-5-oxopyrrolidin-2-yl)methyl)amino)methyl)-4H-pyrido[1,2-a]pyrimidin-4-one;
8-(2,2′-dichloro-3′-(6-methoxy-5-(((((S)-5-oxopyrrolidin-2-yl)methyl)amino)methyl)pyridin-2-yl)-[1,1′-biphenyl]-3-yl)-6-methyl-3-(((((R)-5-oxopyrrolidin-2-yl)methyl)amino)methyl)-4H-pyrido[1,2-a]pyrimidin-4-one;
8-(2,2′-dichloro-3′-(6-methoxy-5-(((((R)-5-oxopyrrolidin-2-yl)methyl)amino)methyl)pyridin-2-yl)-[1,1′-biphenyl]-3-yl)-6-methyl-3-(((((R)-5-oxopyrrolidin-2-yl)methyl)amino)methyl)-4H-pyrido[1,2-a]pyrimidin-4-one;
(S)-2-(((8-(2,2′-dichloro-3′-(6-methoxy-5-((((5-oxopyrrolidin-2-yl)methyl)amino)methyl)pyridin-2-yl)-[1,1′-biphenyl]-3-yl)-4-oxo-4H-pyrido[1,2-a]pyrimidin-3-yl)methyl)amino)-2-methylpropanoic acid;
(R)-2-(((8-(2,2′-dichloro-3′-(6-methoxy-5-((((5-oxopyrrolidin-2-yl)methyl)amino)methyl)pyridin-2-yl)-[1,1′-biphenyl]-3-yl)-4-oxo-4H-pyrido[1,2-a]pyrimidin-3-yl)methyl)amino)-2-methylpropanoic acid;
8-(2,2′-dichloro-3′-(5-((((R)-2-hydroxypropyl)amino)methyl)-6-methoxypyridin-2-yl)-[1,1′-biphenyl]-3-yl)-3-((((R)-2-hydroxypropyl)amino)methyl)-4H-pyrido[1,2-a]pyrimidin-4-one;
8-(2,2′-dichloro-3′-(5-((((S)-2-hydroxypropyl)amino)methyl)-6-methoxypyridin-2-yl)-[1,1′-biphenyl]-3-yl)-3-((((R)-2-hydroxypropyl)amino)methyl)-4H-pyrido[1,2-a]pyrimidin-4-one;
8-(2,2′-dichloro-3′-(5-((((R)-2-hydroxypropyl)amino)methyl)-6-methoxypyridin-2-yl)-[1,1′-biphenyl]-3-yl)-3-((((S)-2-hydroxypropyl)amino)methyl)-4H-pyrido[1,2-a]pyrimidin-4-one;
8-(2,2′-dichloro-3′-(5-((((S)-2-hydroxypropyl)amino)methyl)-6-methoxypyridin-2-yl)-[1,1′-biphenyl]-3-yl)-3-((((S)-2-hydroxypropyl)amino)methyl)-4H-pyrido[1,2-a]pyrimidin-4-one;
S)-N-((8-(2,2′-dichloro-3′-(6-methoxy-5-((((5-oxopyrrolidin-2-yl)methyl)amino)methyl)pyridin-2-yl)-[1,1′-biphenyl]-3-yl)-4-oxo-4H-pyrido[1,2-a]pyrimidin-3-yl)methyl)-N-methylglycine;
(R)—N-((8-(2,2′-dichloro-3′-(6-methoxy-5-((((5-oxopyrrolidin-2-yl)methyl)amino)methyl)pyridin-2-yl)-[1,1′-biphenyl]-3-yl)-4-oxo-4H-pyrido[1,2-a]pyrimidin-3-yl)methyl)-N-methylglycine;
1-((8-(2,2′-dichloro-3′-(5-(((2-hydroxyethyl)amino)methyl)-6-methoxypyridin-2-yl)-[1,1′-biphenyl]-3-yl)-4-oxo-4H-pyrido[1,2-a]pyrimidin-3-yl)methyl)azetidine-3-carboxylic acid;
(S)-((8-(2,2′-dichloro-3′-(6-methoxy-5-((((5-oxopyrrolidin-2-yl)methyl)amino)methyl)pyridin-2-yl)-[1,1′-biphenyl]-3-yl)-4-oxo-4H-pyrido[1,2-a]pyrimidin-3-yl)methyl)glycine;
(R)-((8-(2,2′-dichloro-3′-(6-methoxy-5-((((5-oxopyrrolidin-2-yl)methyl)amino)methyl)pyridin-2-yl)-[1,1′-biphenyl]-3-yl)-4-oxo-4H-pyrido[1,2-a]pyrimidin-3-yl)methyl)glycine;
N-((8-(2,2′-dichloro-3′-(6-methoxy-5-(((((S)-5-oxopyrrolidin-2-yl)methyl)amino)methyl)pyridin-2-yl)-[1,1′-biphenyl]-3-yl)-4-oxo-4H-pyrido[1,2-a]pyrimidin-3-yl)methyl)-N-methyl-L-alanine;
N-((8-(2,2′-dichloro-3′-(6-methoxy-5-(((((R)-5-oxopyrrolidin-2-yl)methyl)amino)methyl)pyridin-2-yl)-[1,1′-biphenyl]-3-yl)-4-oxo-4H-pyrido[1,2-a]pyrimidin-3-yl)methyl)-N-methyl-L-alanine;
N-((8-(2,2′-dichloro-3′-(6-methoxy-5-(((((S)-5-oxopyrrolidin-2-yl)methyl)amino)methyl)pyridin-2-yl)-[1,1′-biphenyl]-3-yl)-4-oxo-4H-pyrido[1,2-a]pyrimidin-3-yl)methyl)-N-methyl-D-alanine;
N-((8-(2,2′-dichloro-3′-(6-methoxy-5-(((((R)-5-oxopyrrolidin-2-yl)methyl)amino)methyl)pyridin-2-yl)-[1,1′-biphenyl]-3-yl)-4-oxo-4H-pyrido[1,2-a]pyrimidin-3-yl)methyl)-N-methyl-D-alanine;
((8-(2,2′-dichloro-3′-(5-(((2-hydroxyethyl)amino)methyl)-6-methoxypyridin-2-yl)-[1,1′-biphenyl]-3-yl)-4-oxo-4H-pyrido[1,2-a]pyrimidin-3-yl)methyl)-L-serine;
((8-(2,2′-dichloro-3′-(5-(((2-hydroxyethyl)amino)methyl)-6-methoxypyridin-2-yl)-[1,1′-biphenyl]-3-yl)-4-oxo-4H-pyrido[1,2-a]pyrimidin-3-yl)methyl)-D-serine;
methyl (S)-1-((8-(2,2′-dichloro-3′-(6-methoxy-5-(((((S)-5-oxopyrrolidin-2-yl)methyl)amino)methyl)pyridin-2-yl)-[1,1′-biphenyl]-3-yl)-4-oxo-4H-pyrido[1,2-a]pyrimidin-3-yl)methyl)pyrrolidine-3-carboxylate;
methyl (R)-1-((8-(2,2′-dichloro-3′-(6-methoxy-5-(((((S)-5-oxopyrrolidin-2-yl)methyl)amino)methyl)pyridin-2-yl)-[1,1′-biphenyl]-3-yl)-4-oxo-4H-pyrido[1,2-a]pyrimidin-3-yl)methyl)pyrrolidine-3-carboxylate;
methyl (S)-1-((8-(2,2′-dichloro-3′-(6-methoxy-5-(((((R)-5-oxopyrrolidin-2-yl)methyl)amino)methyl)pyridin-2-yl)-[1,1′-biphenyl]-3-yl)-4-oxo-4H-pyrido[1,2-a]pyrimidin-3-yl)methyl)pyrrolidine-3-carboxylate;
methyl (R)-1-((8-(2,2′-dichloro-3′-(6-methoxy-5-(((((R)-5-oxopyrrolidin-2-yl)methyl)amino)methyl)pyridin-2-yl)-[1,1′-biphenyl]-3-yl)-4-oxo-4H-pyrido[1,2-a]pyrimidin-3-yl)methyl)pyrrolidine-3-carboxylate;
8-(2,2′-dichloro-3′-(6-methoxy-5-(((((S)-5-oxopyrrolidin-2-yl)methyl)amino)methyl)pyridin-2-yl)-[1,1′-biphenyl]-3-yl)-3-((((S)-2-hydroxypropyl)amino)methyl)-2-methyl-4H-pyrido[1,2-a]pyrimidin-4-one;
8-(2,2′-dichloro-3′-(6-methoxy-5-(((((R)-5-oxopyrrolidin-2-yl)methyl)amino)methyl)pyridin-2-yl)-[1,1′-biphenyl]-3-yl)-3-((((S)-2-hydroxypropyl)amino)methyl)-2-methyl-4H-pyrido[1,2-a]pyrimidin-4-one;
8-(2,2′-dichloro-3′-(6-methoxy-5-(((((S)-5-oxopyrrolidin-2-yl)methyl)amino)methyl)pyridin-2-yl)-[1,1′-biphenyl]-3-yl)-3-((((R)-2-hydroxypropyl)amino)methyl)-2-methyl-4H-pyrido[1,2-a]pyrimidin-4-one;
8-(2,2′-dichloro-3′-(6-methoxy-5-(((((R)-5-oxopyrrolidin-2-yl)methyl)amino)methyl)pyridin-2-yl)-[1,1′-biphenyl]-3-yl)-3-((((R)-2-hydroxypropyl)amino)methyl)-2-methyl-4H-pyrido[1,2-a]pyrimidin-4-one;
(S)-5-((((8-chloro-2-(2,2′-dichloro-3′-(6-methoxy-5-(((((S)-5-oxopyrrolidin-2-yl)methyl)amino)methyl)pyridin-2-yl)-[1,1′-biphenyl]-3-yl)-5-methoxyquinolin-6-yl)methyl)amino)methyl)pyrrolidin-2-one;
(R)-5-((((8-chloro-2-(2,2′-dichloro-3′-(6-methoxy-5-(((((S)-5-oxopyrrolidin-2-yl)methyl)amino)methyl)pyridin-2-yl)-[1,1′-biphenyl]-3-yl)-5-methoxyquinolin-6-yl)methyl)amino)methyl)pyrrolidin-2-one;
(S)-5-((((8-chloro-2-(2,2′-dichloro-3′-(6-methoxy-5-(((((R)-5-oxopyrrolidin-2-yl)methyl)amino)methyl)pyridin-2-yl)-[1,1′-biphenyl]-3-yl)-5-methoxyquinolin-6-yl)methyl)amino)methyl)pyrrolidin-2-one;
(R)-5-((((8-chloro-2-(2,2′-dichloro-3′-(6-methoxy-5-(((((R)-5-oxopyrrolidin-2-yl)methyl)amino)methyl)pyridin-2-yl)-[1,1′-biphenyl]-3-yl)-5-methoxyquinolin-6-yl)methyl)amino)methyl)pyrrolidin-2-one;
8-(2,2′-dichloro-3′-(5-((((R)-2-hydroxypropyl)amino)methyl)-6-methoxypyridin-2-yl)-[1,1′-biphenyl]-3-yl)-3-((((S)-2-hydroxypropyl)amino)methyl)-4H-pyrido[1,2-a]pyrimidin-4-one;
8-(2,2′-dichloro-3′-(5-((((R)-2-hydroxypropyl)amino)methyl)-6-methoxypyridin-2-yl)-[1,1′-biphenyl]-3-yl)-3-((((R)-2-hydroxypropyl)amino)methyl)-4H-pyrido[1,2-a]pyrimidin-4-one;
8-(2,2′-dichloro-3′-(5-((((S)-2-hydroxypropyl)amino)methyl)-6-methoxypyridin-2-yl)-[1,1′-biphenyl]-3-yl)-3-((((R)-2-hydroxypropyl)amino)methyl)-4H-pyrido[1,2-a]pyrimidin-4-one;
8-(2,2′-dichloro-3′-(5-((((R)-2-hydroxypropyl)amino)methyl)-6-methoxypyridin-2-yl)-[1,1′-biphenyl]-3-yl)-3-((((R)-2-hydroxypropyl)amino)methyl)-4H-pyrido[1,2-a]pyrimidin-4-one;
8-(2,2′-dichloro-3′-(5-((((S)-2-hydroxypropyl)amino)methyl)-6-methoxypyridin-2-yl)-[1,1′-biphenyl]-3-yl)-3-(((((S)-5-oxopyrrolidin-2-yl)methyl)amino)methyl)-4H-pyrido[1,2-a]pyrimidin-4-one;
8-(2,2′-dichloro-3′-(5-((((R)-2-hydroxypropyl)amino)methyl)-6-methoxypyridin-2-yl)-[1,1′-biphenyl]-3-yl)-3-(((((S)-5-oxopyrrolidin-2-yl)methyl)amino)methyl)-4H-pyrido[1,2-a]pyrimidin-4-one;
8-(2,2′-dichloro-3′-(5-((((S)-2-hydroxypropyl)amino)methyl)-6-methoxypyridin-2-yl)-[1,1′-biphenyl]-3-yl)-3-(((((R)-5-oxopyrrolidin-2-yl)methyl)amino)methyl)-4H-pyrido[1,2-a]pyrimidin-4-one;
8-(2,2′-dichloro-3′-(5-((((R)-2-hydroxypropyl)amino)methyl)-6-methoxypyridin-2-yl)-[1,1′-biphenyl]-3-yl)-3-(((((R)-5-oxopyrrolidin-2-yl)methyl)amino)methyl)-4H-pyrido[1,2-a]pyrimidin-4-one;
8-(2,2′-dichloro-3′-(5-((((R)-2-hydroxypropyl)amino)methyl)-6-methoxypyridin-2-yl)-[1,1′-biphenyl]-3-yl)-3-(((((S)-5-oxopyrrolidin-2-yl)methyl)amino)methyl)-4H-pyrido[1,2-a]pyrimidin-4-one;
8-(2,2′-dichloro-3′-(5-((((R)-2-hydroxypropyl)amino)methyl)-6-methoxypyridin-2-yl)-[1,1′-biphenyl]-3-yl)-3-(((((R)-5-oxopyrrolidin-2-yl)methyl)amino)methyl)-4H-pyrido[1,2-a]pyrimidin-4-one;
8-(2,2′-dichloro-3′-(5-((((S)-2-hydroxypropyl)amino)methyl)-6-methoxypyridin-2-yl)-[1,1′-biphenyl]-3-yl)-3-(((((S)-5-oxopyrrolidin-2-yl)methyl)amino)methyl)-4H-pyrido[1,2-a]pyrimidin-4-one;
8-(2,2′-dichloro-3′-(5-((((S)-2-hydroxypropyl)amino)methyl)-6-methoxypyridin-2-yl)-[1,1′-biphenyl]-3-yl)-3-(((((R)-5-oxopyrrolidin-2-yl)methyl)amino)methyl)-4H-pyrido[1,2-a]pyrimidin-4-one;
2-(8-(2,2′-dichloro-3′-(6-methoxy-5-(((((S)-5-oxopyrrolidin-2-yl)methyl)amino)methyl)pyridin-2-yl)-[1,1′-biphenyl]-3-yl)-4-oxo-4H-pyrido[1,2-a]pyrimidin-3-yl)-3,4,5,6-tetrahydropyrimidine-(4S)-carboxylic acid;
2-(8-(2,2′-dichloro-3′-(6-methoxy-5-(((((S)-5-oxopyrrolidin-2-yl)methyl)amino)methyl)pyridin-2-yl)-[1,1′-biphenyl]-3-yl)-4-oxo-4H-pyrido[1,2-a]pyrimidin-3-yl)-3,4,5,6-tetrahydropyrimidine-(4R)-carboxylic acid;
2-(8-(2,2′-dichloro-3′-(6-methoxy-5-(((((R)-5-oxopyrrolidin-2-yl)methyl)amino)methyl)pyridin-2-yl)-[1,1′-biphenyl]-3-yl)-4-oxo-4H-pyrido[1,2-a]pyrimidin-3-yl)-3,4,5,6-tetrahydropyrimidine-(4S)-carboxylic acid;
2-(8-(2,2′-dichloro-3′-(6-methoxy-5-(((((R)-5-oxopyrrolidin-2-yl)methyl)amino)methyl)pyridin-2-yl)-[1,1′-biphenyl]-3-yl)-4-oxo-4H-pyrido[1,2-a]pyrimidin-3-yl)-3,4,5,6-tetrahydropyrimidine-(4R)-carboxylic acid;
(S)-2-(((8-(2,2′-dichloro-3′-(6-methoxy-5-((((5-oxopyrrolidin-2-yl)methyl)amino)methyl)pyridin-2-yl)-[1,1′-biphenyl]-3-yl)-4-oxo-4H-pyrido[1,2-a]pyrimidin-3-yl)methyl)(methyl)amino)-N-methylacetamide;
(R)-2-(((8-(2,2′-dichloro-3′-(6-methoxy-5-((((5-oxopyrrolidin-2-yl)methyl)amino)methyl)pyridin-2-yl)-[1,1′-biphenyl]-3-yl)-4-oxo-4H-pyrido[1,2-a]pyrimidin-3-yl)methyl)(methyl)amino)-N-methylacetamide;
8-(2,2′-dichloro-3′-(6-methoxy-5-(((((S)-5-oxopyrrolidin-2-yl)methyl)amino)methyl)pyridin-2-yl)-[1,1′-biphenyl]-3-yl)-2-methoxy-3-(((((S)-5-oxopyrrolidin-2-yl)methyl)amino)methyl)-4H-pyrido[1,2-a]pyrimidin-4-one;
8-(2,2′-dichloro-3′-(6-methoxy-5-(((((R)-5-oxopyrrolidin-2-yl)methyl)amino)methyl)pyridin-2-yl)-[1,1′-biphenyl]-3-yl)-2-methoxy-3-(((((S)-5-oxopyrrolidin-2-yl)methyl)amino)methyl)-4H-pyrido[1,2-a]pyrimidin-4-one;
8-(2,2′-dichloro-3′-(6-methoxy-5-(((((S)-5-oxopyrrolidin-2-yl)methyl)amino)methyl)pyridin-2-yl)-[1,1′-biphenyl]-3-yl)-2-methoxy-3-(((((R)-5-oxopyrrolidin-2-yl)methyl)amino)methyl)-4H-pyrido[1,2-a]pyrimidin-4-one;
8-(2,2′-dichloro-3′-(6-methoxy-5-(((((R)-5-oxopyrrolidin-2-yl)methyl)amino)methyl)pyridin-2-yl)-[1,1′-biphenyl]-3-yl)-2-methoxy-3-(((((R)-5-oxopyrrolidin-2-yl)methyl)amino)methyl)-4H-pyrido[1,2-a]pyrimidin-4-one;
(S)-2-(((8-(2,2′-dichloro-3′-(6-methoxy-5-((((5-oxopyrrolidin-2-yl)methyl)amino)methyl)pyridin-2-yl)-[1,1′-biphenyl]-3-yl)-4-oxo-4H-pyrido[1,2-a]pyrimidin-3-yl)methyl)amino)-N-methylacetamide;
(R)-2-(((8-(2,2′-dichloro-3′-(6-methoxy-5-((((5-oxopyrrolidin-2-yl)methyl)amino)methyl)pyridin-2-yl)-[1,1′-biphenyl]-3-yl)-4-oxo-4H-pyrido[1,2-a]pyrimidin-3-yl)methyl)amino)-N-methylacetamide;
8-(2,2′-dichloro-3′-(8-chloro-5-methoxy-6-(((((S)-5-oxopyrrolidin-2-yl)methyl)amino)methyl)quinolin-2-yl)-[1,1′-biphenyl]-3-yl)-3-(((((S)-5-oxopyrrolidin-2-yl)methyl)amino)methyl)-4H-pyrido[1,2-a]pyrimidin-4-one;
8-(2,2′-dichloro-3′-(8-chloro-5-methoxy-6-(((((R)-5-oxopyrrolidin-2-yl)methyl)amino)methyl)quinolin-2-yl)-[1,1′-biphenyl]-3-yl)-3-(((((S)-5-oxopyrrolidin-2-yl)methyl)amino)methyl)-4H-pyrido[1,2-a]pyrimidin-4-one;
8-(2,2′-dichloro-3′-(8-chloro-5-methoxy-6-(((((S)-5-oxopyrrolidin-2-yl)methyl)amino)methyl)quinolin-2-yl)-[1,1′-biphenyl]-3-yl)-3-(((((R)-5-oxopyrrolidin-2-yl)methyl)amino)methyl)-4H-pyrido[1,2-a]pyrimidin-4-one;
8-(2,2′-dichloro-3′-(8-chloro-5-methoxy-6-(((((R)-5-oxopyrrolidin-2-yl)methyl)amino)methyl)quinolin-2-yl)-[1,1′-biphenyl]-3-yl)-3-(((((R)-5-oxopyrrolidin-2-yl)methyl)amino)methyl)-4H-pyrido[1,2-a]pyrimidin-4-one;
8-(2,2′-dichloro-3′-(6-methoxy-5-(((((S)-5-oxopyrrolidin-2-yl)methyl)amino)methyl)pyridin-2-yl)-[1,1′-biphenyl]-3-yl)-3-((((1r,3r)-3-hydroxy-3-methylcyclobutyl)amino)methyl)-2-methyl-4H-pyrido[1,2-a]pyrimidin-4-one;
8-(2,2′-dichloro-3′-(6-methoxy-5-(((((S)-5-oxopyrrolidin-2-yl)methyl)amino)methyl)pyridin-2-yl)-[1,1′-biphenyl]-3-yl)-3-((((1s,3s)-3-hydroxy-3-methylcyclobutyl)amino)methyl)-2-methyl-4H-pyrido[1,2-a]pyrimidin-4-one;
8-(2,2′-dichloro-3′-(6-methoxy-5-(((((R)-5-oxopyrrolidin-2-yl)methyl)amino)methyl)pyridin-2-yl)-[1,1′-biphenyl]-3-yl)-3-((((1r,3r)-3-hydroxy-3-methylcyclobutyl)amino)methyl)-2-methyl-4H-pyrido[1,2-a]pyrimidin-4-one;
8-(2,2′-dichloro-3′-(6-methoxy-5-(((((R)-5-oxopyrrolidin-2-yl)methyl)amino)methyl)pyridin-2-yl)-[1,1′-biphenyl]-3-yl)-3-((((1s,3s)-3-hydroxy-3-methylcyclobutyl)amino)methyl)-2-methyl-4H-pyrido[1,2-a]pyrimidin-4-one;
8-(2,2′-dichloro-3′-(5-((((1r,3r)-3-hydroxy-3-methylcyclobutyl)amino)methyl)-6-methoxypyridin-2-yl)-[1,1′-biphenyl]-3-yl)-3-((((1r,3r)-3-hydroxy-3-methylcyclobutyl)amino)methyl)-2-methyl-4H-pyrido[1,2-a]pyrimidin-4-one;
8-(2,2′-dichloro-3′-(5-((((1s,3s)-3-hydroxy-3-methylcyclobutyl)amino)methyl)-6-methoxypyridin-2-yl)-[1,1′-biphenyl]-3-yl)-3-((((1r,3r)-3-hydroxy-3-methylcyclobutyl)amino)methyl)-2-methyl-4H-pyrido[1,2-a]pyrimidin-4-one;
8-(2,2′-dichloro-3′-(5-((((1r,3r)-3-hydroxy-3-methylcyclobutyl)amino)methyl)-6-methoxypyridin-2-yl)-[1,1′-biphenyl]-3-yl)-3-((((1s,3s)-3-hydroxy-3-methylcyclobutyl)amino)methyl)-2-methyl-4H-pyrido[1,2-a]pyrimidin-4-one;
8-(2,2′-dichloro-3′-(5-((((1s,3s)-3-hydroxy-3-methylcyclobutyl)amino)methyl)-6-methoxypyridin-2-yl)-[1,1′-biphenyl]-3-yl)-3-((((1s,3s)-3-hydroxy-3-methylcyclobutyl)amino)methyl)-2-methyl-4H-pyrido[1,2-a]pyrimidin-4-one;
8-(2,2′-dichloro-3′-(6-methoxy-5-(((((S)-5-oxopyrrolidin-2-yl)methyl)amino)methyl)pyridin-2-yl)-[1,1′-biphenyl]-3-yl)-3-(((((6S)-oxopiperidin-2-yl)methyl)amino)methyl)-4H-pyrido[1,2-a]pyrimidin-4-one;
8-(2,2′-dichloro-3′-(6-methoxy-5-(((((R)-5-oxopyrrolidin-2-yl)methyl)amino)methyl)pyridin-2-yl)-[1,1′-biphenyl]-3-yl)-3-(((((6S)-oxopiperidin-2-yl)methyl)amino)methyl)-4H-pyrido[1,2-a]pyrimidin-4-one;
8-(2,2′-dichloro-3′-(6-methoxy-5-(((((S)-5-oxopyrrolidin-2-yl)methyl)amino)methyl)pyridin-2-yl)-[1,1′-biphenyl]-3-yl)-3-(((((6R)-oxopiperidin-2-yl)methyl)amino)methyl)-4H-pyrido[1,2-a]pyrimidin-4-one;
8-(2,2′-dichloro-3′-(6-methoxy-5-(((((R)-5-oxopyrrolidin-2-yl)methyl)amino)methyl)pyridin-2-yl)-[1,1′-biphenyl]-3-yl)-3-(((((6R)-oxopiperidin-2-yl)methyl)amino)methyl)-4H-pyrido[1,2-a]pyrimidin-4-one;
(S)-8-(2,2′-dichloro-3′-(6-methoxy-5-((((5-oxopyrrolidin-2-yl)methyl)amino)methyl)pyridin-2-yl)-[1,1′-biphenyl]-3-yl)-3-(((2-(2-oxoimidazolidin-1-yl)ethyl)amino)methyl)-4H-pyrido[1,2-a]pyrimidin-4-one;
(R)-8-(2,2′-dichloro-3′-(6-methoxy-5-((((5-oxopyrrolidin-2-yl)methyl)amino)methyl)pyridin-2-yl)-[1,1′-biphenyl]-3-yl)-3-(((2-(2-oxoimidazolidin-1-yl)ethyl)amino)methyl)-4H-pyrido[1,2-a]pyrimidin-4-one;
8-(2′-chloro-3′-(6-methoxy-5-(((((S)-5-oxopyrrolidin-2-yl)methyl)amino)methyl)pyridin-2-yl)-2-(trifluoromethyl)-[1,1′-biphenyl]-3-yl)-3-(((((S)-5-oxopyrrolidin-2-yl)methyl)amino)methyl)-4H-pyrido[1,2-a]pyrimidin-4-one;
8-(2′-chloro-3′-(6-methoxy-5-(((((R)-5-oxopyrrolidin-2-yl)methyl)amino)methyl)pyridin-2-yl)-2-(trifluoromethyl)-[1,1′-biphenyl]-3-yl)-3-(((((S)-5-oxopyrrolidin-2-yl)methyl)amino)methyl)-4H-pyrido[1,2-a]pyrimidin-4-one;
8-(2′-chloro-3′-(6-methoxy-5-(((((S)-5-oxopyrrolidin-2-yl)methyl)amino)methyl)pyridin-2-yl)-2-(trifluoromethyl)-[1,1′-biphenyl]-3-yl)-3-(((((R)-5-oxopyrrolidin-2-yl)methyl)amino)methyl)-4H-pyrido[1,2-a]pyrimidin-4-one;
8-(2′-chloro-3′-(6-methoxy-5-(((((R)-5-oxopyrrolidin-2-yl)methyl)amino)methyl)pyridin-2-yl)-2-(trifluoromethyl)-[1,1′-biphenyl]-3-yl)-3-(((((R)-5-oxopyrrolidin-2-yl)methyl)amino)methyl)-4H-pyrido[1,2-a]pyrimidin-4-one;
(S)-2-(((8-(2,2′-dichloro-3′-(6-methoxy-5-((((5-oxopyrrolidin-2-yl)methyl)amino)methyl)pyridin-2-yl)-[1,1′-biphenyl]-3-yl)-4-oxo-4H-pyrido[1,2-a]pyrimidin-3-yl)methyl)amino)-N,2-dimethylpropanamide;
(R)-2-(((8-(2,2′-dichloro-3′-(6-methoxy-5-((((5-oxopyrrolidin-2-yl)methyl)amino)methyl)pyridin-2-yl)-[1,1′-biphenyl]-3-yl)-4-oxo-4H-pyrido[1,2-a]pyrimidin-3-yl)methyl)amino)-N,2-dimethylpropanamide;
(S)-3-(((8-(2,2′-dichloro-3′-(6-methoxy-5-((((5-oxopyrrolidin-2-yl)methyl)amino)methyl)pyridin-2-yl)-[1,1′-biphenyl]-3-yl)-4-oxo-4H-pyrido[1,2-a]pyrimidin-3-yl)methyl)amino)-N-methylpropanamide;
(R)-3-(((8-(2,2′-dichloro-3′-(6-methoxy-5-((((5-oxopyrrolidin-2-yl)methyl)amino)methyl)pyridin-2-yl)-[1,1′-biphenyl]-3-yl)-4-oxo-4H-pyrido[1,2-a]pyrimidin-3-yl)methyl)amino)-N-methylpropanamide;
8-(2,2′-dichloro-3′-(6-methoxy-5-(((((S)-5-oxopyrrolidin-2-yl)methyl)amino)methyl)pyridin-2-yl)-[1,1′-biphenyl]-3-yl)-3-((((1r,3r)-3-hydroxy-3-methylcyclobutyl)amino)methyl)-6-methyl-4H-pyrido[1,2-a]pyrimidin-4-one;
8-(2,2′-dichloro-3′-(6-methoxy-5-(((((S)-5-oxopyrrolidin-2-yl)methyl)amino)methyl)pyridin-2-yl)-[1,1′-biphenyl]-3-yl)-3-((((1s,3s)-3-hydroxy-3-methylcyclobutyl)amino)methyl)-6-methyl-4H-pyrido[1,2-a]pyrimidin-4-one;
8-(2,2′-dichloro-3′-(6-methoxy-5-(((((R)-5-oxopyrrolidin-2-yl)methyl)amino)methyl)pyridin-2-yl)-[1,1′-biphenyl]-3-yl)-3-((((1r,3r)-3-hydroxy-3-methylcyclobutyl)amino)methyl)-6-methyl-4H-pyrido[1,2-a]pyrimidin-4-one;
8-(2,2′-dichloro-3′-(6-methoxy-5-(((((R)-5-oxopyrrolidin-2-yl)methyl)amino)methyl)pyridin-2-yl)-[1,1′-biphenyl]-3-yl)-3-((((1s,3s)-3-hydroxy-3-methylcyclobutyl)amino)methyl)-6-methyl-4H-pyrido[1,2-a]pyrimidin-4-one;
7-(2,2′-dichloro-3′-(6-methoxy-5-(((((S)-5-oxopyrrolidin-2-yl)methyl)amino)methyl)pyridin-2-yl)-[1,1′-biphenyl]-3-yl)-2-(((((S)-5-oxopyrrolidin-2-yl)methyl)amino)methyl)-4H-pyrido[1,2-a]pyrimidin-4-one;
7-(2,2′-dichloro-3′-(6-methoxy-5-(((((R)-5-oxopyrrolidin-2-yl)methyl)amino)methyl)pyridin-2-yl)-[1,1′-biphenyl]-3-yl)-2-(((((S)-5-oxopyrrolidin-2-yl)methyl)amino)methyl)-4H-pyrido[1,2-a]pyrimidin-4-one;
7-(2,2′-dichloro-3′-(6-methoxy-5-(((((S)-5-oxopyrrolidin-2-yl)methyl)amino)methyl)pyridin-2-yl)-[1,1′-biphenyl]-3-yl)-2-(((((R)-5-oxopyrrolidin-2-yl)methyl)amino)methyl)-4H-pyrido[1,2-a]pyrimidin-4-one;
7-(2,2′-dichloro-3′-(6-methoxy-5-(((((R)-5-oxopyrrolidin-2-yl)methyl)amino)methyl)pyridin-2-yl)-[1,1′-biphenyl]-3-yl)-2-(((((R)-5-oxopyrrolidin-2-yl)methyl)amino)methyl)-4H-pyrido[1,2-a]pyrimidin-4-one;
8-(2-chloro-3-((S)-1-((3-chloro-6-methoxy-5-(((((S)-5-oxopyrrolidin-2-yl)methyl)amino)methyl)pyridin-2-yl)methyl)-2,3-dihydro-1H-inden-4-yl)phenyl)-3-(((((S)-5-oxopyrrolidin-2-yl)methyl)amino)methyl)-4H-pyrido[1,2-a]pyrimidin-4-one;
8-(2-chloro-3-((S)-1-((3-chloro-6-methoxy-5-(((((R)-5-oxopyrrolidin-2-yl)methyl)amino)methyl)pyridin-2-yl)methyl)-2,3-dihydro-1H-inden-4-yl)phenyl)-3-(((((S)-5-oxopyrrolidin-2-yl)methyl)amino)methyl)-4H-pyrido[1,2-a]pyrimidin-4-one;
8-(2-chloro-3-((S)-1-((3-chloro-6-methoxy-5-(((((S)-5-oxopyrrolidin-2-yl)methyl)amino)methyl)pyridin-2-yl)methyl)-2,3-dihydro-1H-inden-4-yl)phenyl)-3-(((((R)-5-oxopyrrolidin-2-yl)methyl)amino)methyl)-4H-pyrido[1,2-a]pyrimidin-4-one;
8-(2-chloro-3-((S)-1-((3-chloro-6-methoxy-5-(((((R)-5-oxopyrrolidin-2-yl)methyl)amino)methyl)pyridin-2-yl)methyl)-2,3-dihydro-1H-inden-4-yl)phenyl)-3-(((((R)-5-oxopyrrolidin-2-yl)methyl)amino)methyl)-4H-pyrido[1,2-a]pyrimidin-4-one;
8-(2-chloro-3-((R)-1-((3-chloro-6-methoxy-5-(((((S)-5-oxopyrrolidin-2-yl)methyl)amino)methyl)pyridin-2-yl)methyl)-2,3-dihydro-1H-inden-4-yl)phenyl)-3-(((((S)-5-oxopyrrolidin-2-yl)methyl)amino)methyl)-4H-pyrido[1,2-a]pyrimidin-4-one;
8-(2-chloro-3-((R)-1-((3-chloro-6-methoxy-5-(((((R)-5-oxopyrrolidin-2-yl)methyl)amino)methyl)pyridin-2-yl)methyl)-2,3-dihydro-1H-inden-4-yl)phenyl)-3-(((((S)-5-oxopyrrolidin-2-yl)methyl)amino)methyl)-4H-pyrido[1,2-a]pyrimidin-4-one;
8-(2-chloro-3-((R)-1-((3-chloro-6-methoxy-5-(((((S)-5-oxopyrrolidin-2-yl)methyl)amino)methyl)pyridin-2-yl)methyl)-2,3-dihydro-1H-inden-4-yl)phenyl)-3-(((((R)-5-oxopyrrolidin-2-yl)methyl)amino)methyl)-4H-pyrido[1,2-a]pyrimidin-4-one;
8-(2-chloro-3-((R)-1-((3-chloro-6-methoxy-5-(((((R)-5-oxopyrrolidin-2-yl)methyl)amino)methyl)pyridin-2-yl)methyl)-2,3-dihydro-1H-inden-4-yl)phenyl)-3-(((((R)-5-oxopyrrolidin-2-yl)methyl)amino)methyl)-4H-pyrido[1,2-a]pyrimidin-4-one;
8-(2,2′-dichloro-3′-(6-methoxy-5-(((((S)-5-oxopyrrolidin-2-yl)methyl)amino)methyl)pyridin-2-yl)-[1,1′-biphenyl]-3-yl)-3-(((2-((S)-5-oxopyrrolidin-2-yl)ethyl)amino)methyl)-4H-pyrido[1,2-a]pyrimidin-4-one;
8-(2,2′-dichloro-3′-(6-methoxy-5-(((((R)-5-oxopyrrolidin-2-yl)methyl)amino)methyl)pyridin-2-yl)-[1,1′-biphenyl]-3-yl)-3-(((2-((S)-5-oxopyrrolidin-2-yl)ethyl)amino)methyl)-4H-pyrido[1,2-a]pyrimidin-4-one;
8-(2,2′-dichloro-3′-(6-methoxy-5-(((((S)-5-oxopyrrolidin-2-yl)methyl)amino)methyl)pyridin-2-yl)-[1,1′-biphenyl]-3-yl)-3-(((2-((R)-5-oxopyrrolidin-2-yl)ethyl)amino)methyl)-4H-pyrido[1,2-a]pyrimidin-4-one;
8-(2,2′-dichloro-3′-(6-methoxy-5-(((((R)-5-oxopyrrolidin-2-yl)methyl)amino)methyl)pyridin-2-yl)-[1,1′-biphenyl]-3-yl)-3-(((2-((R)-5-oxopyrrolidin-2-yl)ethyl)amino)methyl)-4H-pyrido[1,2-a]pyrimidin-4-one;
(S)-3-(((2-(1H-imidazol-4-yl)ethyl)amino)methyl)-8-(2,2′-dichloro-3′-(6-methoxy-5-((((5-oxopyrrolidin-2-yl)methyl)amino)methyl)pyridin-2-yl)-[1,1′-biphenyl]-3-yl)-4H-pyrido[1,2-a]pyrimidin-4-one;
(R)-3-(((2-(1H-imidazol-4-yl)ethyl)amino)methyl)-8-(2,2′-dichloro-3′-(6-methoxy-5-((((5-oxopyrrolidin-2-yl)methyl)amino)methyl)pyridin-2-yl)-[1,1′-biphenyl]-3-yl)-4H-pyrido[1,2-a]pyrimidin-4-one;
(S)-3-(((2-(4H-1,2,4-triazol-4-yl)ethyl)amino)methyl)-8-(2,2′-dichloro-3′-(6-methoxy-5-((((5-oxopyrrolidin-2-yl)methyl)amino)methyl)pyridin-2-yl)-[1,1′-biphenyl]-3-yl)-4H-pyrido[1,2-a]pyrimidin-4-one;
(R)-3-(((2-(4H-1,2,4-triazol-4-yl)ethyl)amino)methyl)-8-(2,2′-dichloro-3′-(6-methoxy-5-((((5-oxopyrrolidin-2-yl)methyl)amino)methyl)pyridin-2-yl)-[1,1′-biphenyl]-3-yl)-4H-pyrido[1,2-a]pyrimidin-4-one;
(S)-5-((((6-(3′-(3-amino-2-(hydroxymethyl)thieno[3,2-b]pyridin-6-yl)-2,2′-dichloro-[1,1′-biphenyl]-3-yl)-2-methoxypyridin-3-yl)methyl)amino)methyl)pyrrolidin-2-one;
(R)-5-((((6-(3′-(3-amino-2-(hydroxymethyl)thieno[3,2-b]pyridin-6-yl)-2,2′-dichloro-[1,1′-biphenyl]-3-yl)-2-methoxypyridin-3-yl)methyl)amino)methyl)pyrrolidin-2-one;
(S)-5-((((6-(2,2′-dichloro-3′-(2,3,4,5-tetrahydropyrido[3,2-f][1,4]oxazepin-8-yl)-[1,1′-biphenyl]-3-yl)-2-methoxypyridin-3-yl)methyl)amino)methyl)pyrrolidin-2-one;
(R)-5-((((6-(2,2′-dichloro-3′-(2,3,4,5-tetrahydropyrido[3,2-f][1,4]oxazepin-8-yl)-[1,1′-biphenyl]-3-yl)-2-methoxypyridin-3-yl)methyl)amino)methyl)pyrrolidin-2-one;
(S)-8-(2,2′-dichloro-3′-(6-methoxy-5-((((5-oxopyrrolidin-2-yl)methyl)amino)methyl)pyridin-2-yl)-[1,1′-biphenyl]-3-yl)-3-(((2-(2-oxopyrrolidin-1-yl)ethyl)amino)methyl)-4H-pyrido[1,2-a]pyrimidin-4-one;
(R)-8-(2,2′-dichloro-3′-(6-methoxy-5-((((5-oxopyrrolidin-2-yl)methyl)amino)methyl)pyridin-2-yl)-[1,1′-biphenyl]-3-yl)-3-(((2-(2-oxopyrrolidin-1-yl)ethyl)amino)methyl)-4H-pyrido[1,2-a]pyrimidin-4-one;
methyl (S)-4-(((8-(2,2′-dichloro-3′-(6-methoxy-5-(((((S)-5-oxopyrrolidin-2-yl)methyl)amino)methyl)pyridin-2-yl)-[1,1′-biphenyl]-3-yl)-4-oxo-4H-pyrido[1,2-a]pyrimidin-3-yl)methyl)amino)-3-hydroxybutanoate;
methyl (R)-4-(((8-(2,2′-dichloro-3′-(6-methoxy-5-(((((S)-5-oxopyrrolidin-2-yl)methyl)amino)methyl)pyridin-2-yl)-[1,1′-biphenyl]-3-yl)-4-oxo-4H-pyrido[1,2-a]pyrimidin-3-yl)methyl)amino)-3-hydroxybutanoate;
methyl (S)-4-(((8-(2,2′-dichloro-3′-(6-methoxy-5-(((((R)-5-oxopyrrolidin-2-yl)methyl)amino)methyl)pyridin-2-yl)-[1,1′-biphenyl]-3-yl)-4-oxo-4H-pyrido[1,2-a]pyrimidin-3-yl)methyl)amino)-3-hydroxybutanoate;
methyl (R)-4-(((8-(2,2′-dichloro-3′-(6-methoxy-5-(((((R)-5-oxopyrrolidin-2-yl)methyl)amino)methyl)pyridin-2-yl)-[1,1′-biphenyl]-3-yl)-4-oxo-4H-pyrido[1,2-a]pyrimidin-3-yl)methyl)amino)-3-hydroxybutanoate;
7-(2,2′-dichloro-3′-(6-methoxy-5-(((((S)-5-oxopyrrolidin-2-yl)methyl)amino)methyl)pyridin-2-yl)-[1,1′-biphenyl]-3-yl)-2-((((1r,3r)-3-hydroxycyclobutyl)amino)methyl)-4H-pyrido[1,2-a]pyrimidin-4-one;
7-(2,2′-dichloro-3′-(6-methoxy-5-(((((R)-5-oxopyrrolidin-2-yl)methyl)amino)methyl)pyridin-2-yl)-[1,1′-biphenyl]-3-yl)-2-((((1r,3r)-3-hydroxycyclobutyl)amino)methyl)-4H-pyrido[1,2-a]pyrimidin-4-one;
7-(2,2′-dichloro-3′-(6-methoxy-5-(((((S)-5-oxopyrrolidin-2-yl)methyl)amino)methyl)pyridin-2-yl)-[1,1′-biphenyl]-3-yl)-2-((((1s,3s)-3-hydroxycyclobutyl)amino)methyl)-4H-pyrido[1,2-a]pyrimidin-4-one;
7-(2,2′-dichloro-3′-(6-methoxy-5-(((((R)-5-oxopyrrolidin-2-yl)methyl)amino)methyl)pyridin-2-yl)-[1,1′-biphenyl]-3-yl)-2-((((1s,3s)-3-hydroxycyclobutyl)amino)methyl)-4H-pyrido[1,2-a]pyrimidin-4-one;
(S)-7-(2,2′-dichloro-3′-(6-methoxy-5-((((5-oxopyrrolidin-2-yl)methyl)amino)methyl)pyridin-2-yl)-[1,1′-biphenyl]-3-yl)-2-((3-hydroxy-3-methylazetidin-1-yl)methyl)-4H-pyrido[1,2-a]pyrimidin-4-one;
(R)-7-(2,2′-dichloro-3′-(6-methoxy-5-((((5-oxopyrrolidin-2-yl)methyl)amino)methyl)pyridin-2-yl)-[1,1′-biphenyl]-3-yl)-2-((3-hydroxy-3-methylazetidin-1-yl)methyl)-4H-pyrido[1,2-a]pyrimidin-4-one;
(S)-8-(2,2′-dichloro-3′-(6-methoxy-5-((((5-oxopyrrolidin-2-yl)methyl)amino)methyl)pyridin-2-yl)-[1,1′-biphenyl]-3-yl)-3-(((4,4,5,5,5-pentafluoropentyl)amino)methyl)-4H-pyrido[1,2-a]pyrimidin-4-one;
(R)-8-(2,2′-dichloro-3′-(6-methoxy-5-((((5-oxopyrrolidin-2-yl)methyl)amino)methyl)pyridin-2-yl)-[1,1′-biphenyl]-3-yl)-3-(((4,4,5,5,5-pentafluoropentyl)amino)methyl)-4H-pyrido[1,2-a]pyrimidin-4-one;
(S)-8-(2,2′-dichloro-3′-(6-methoxy-5-((((5-oxopyrrolidin-2-yl)methyl)amino)methyl)pyridin-2-yl)-[1,1′-biphenyl]-3-yl)-3-(((1,1-dioxidotetrahydro-2H-thiopyran-4-yl)amino)methyl)-4H-pyrido[1,2-a]pyrimidin-4-one;
(R)-8-(2,2′-dichloro-3′-(6-methoxy-5-((((5-oxopyrrolidin-2-yl)methyl)amino)methyl)pyridin-2-yl)-[1,1′-biphenyl]-3-yl)-3-(((1,1-dioxidotetrahydro-2H-thiopyran-4-yl)amino)methyl)-4H-pyrido[1,2-a]pyrimidin-4-one;
8-(2,2′-dichloro-3′-(6-methoxy-5-(((((S)-5-oxopyrrolidin-2-yl)methyl)amino)methyl)pyridin-2-yl)-[1,1′-biphenyl]-3-yl)-3-(((((1S,2S)-2-(hydroxymethyl)cyclopropyl)methyl)amino)methyl)-4H-pyrido[1,2-a]pyrimidin-4-one;
8-(2,2′-dichloro-3′-(6-methoxy-5-(((((S)-5-oxopyrrolidin-2-yl)methyl)amino)methyl)pyridin-2-yl)-[1,1′-biphenyl]-3-yl)-3-(((((1R,2S)-2-(hydroxymethyl)cyclopropyl)methyl)amino)methyl)-4H-pyrido[1,2-a]pyrimidin-4-one;
8-(2,2′-dichloro-3′-(6-methoxy-5-(((((S)-5-oxopyrrolidin-2-yl)methyl)amino)methyl)pyridin-2-yl)-[1,1′-biphenyl]-3-yl)-3-(((((1S,2R)-2-(hydroxymethyl)cyclopropyl)methyl)amino)methyl)-4H-pyrido[1,2-a]pyrimidin-4-one;
8-(2,2′-dichloro-3′-(6-methoxy-5-(((((S)-5-oxopyrrolidin-2-yl)methyl)amino)methyl)pyridin-2-yl)-[1,1′-biphenyl]-3-yl)-3-(((((1R,2R)-2-(hydroxymethyl)cyclopropyl)methyl)amino)methyl)-4H-pyrido[1,2-a]pyrimidin-4-one;
8-(2,2′-dichloro-3′-(6-methoxy-5-(((((R)-5-oxopyrrolidin-2-yl)methyl)amino)methyl)pyridin-2-yl)-[1,1′-biphenyl]-3-yl)-3-(((((1S,2S)-2-(hydroxymethyl)cyclopropyl)methyl)amino)methyl)-4H-pyrido[1,2-a]pyrimidin-4-one;
8-(2,2′-dichloro-3′-(6-methoxy-5-(((((R)-5-oxopyrrolidin-2-yl)methyl)amino)methyl)pyridin-2-yl)-[1,1′-biphenyl]-3-yl)-3-(((((1R,2S)-2-(hydroxymethyl)cyclopropyl)methyl)amino)methyl)-4H-pyrido[1,2-a]pyrimidin-4-one;
8-(2,2′-dichloro-3′-(6-methoxy-5-(((((R)-5-oxopyrrolidin-2-yl)methyl)amino)methyl)pyridin-2-yl)-[1,1′-biphenyl]-3-yl)-3-(((((1S,2R)-2-(hydroxymethyl)cyclopropyl)methyl)amino)methyl)-4H-pyrido[1,2-a]pyrimidin-4-one;
8-(2,2′-dichloro-3′-(6-methoxy-5-(((((R)-5-oxopyrrolidin-2-yl)methyl)amino)methyl)pyridin-2-yl)-[1,1′-biphenyl]-3-yl)-3-(((((1R,2R)-2-(hydroxymethyl)cyclopropyl)methyl)amino)methyl)-4H-pyrido[1,2-a]pyrimidin-4-one;
8-(2,2′-dichloro-3′-(6-methoxy-5-(((((S)-5-oxopyrrolidin-2-yl)methyl)amino)methyl)pyridin-2-yl)-[1,1′-biphenyl]-3-yl)-3-((((S)-2-hydroxypropyl)amino)methyl)-6-methyl-4H-pyrido[1,2-a]pyrimidin-4-one;
8-(2,2′-dichloro-3′-(6-methoxy-5-(((((R)-5-oxopyrrolidin-2-yl)methyl)amino)methyl)pyridin-2-yl)-[1,1′-biphenyl]-3-yl)-3-((((S)-2-hydroxypropyl)amino)methyl)-6-methyl-4H-pyrido[1,2-a]pyrimidin-4-one;
8-(2,2′-dichloro-3′-(6-methoxy-5-(((((S)-5-oxopyrrolidin-2-yl)methyl)amino)methyl)pyridin-2-yl)-[1,1′-biphenyl]-3-yl)-3-((((R)-2-hydroxypropyl)amino)methyl)-6-methyl-4H-pyrido[1,2-a]pyrimidin-4-one;
8-(2,2′-dichloro-3′-(6-methoxy-5-(((((R)-5-oxopyrrolidin-2-yl)methyl)amino)methyl)pyridin-2-yl)-[1,1′-biphenyl]-3-yl)-3-((((R)-2-hydroxypropyl)amino)methyl)-6-methyl-4H-pyrido[1,2-a]pyrimidin-4-one;
2-chloro-8-(2,2′-dichloro-3′-(6-methoxy-5-(((((S)-5-oxopyrrolidin-2-yl)methyl)amino)methyl)pyridin-2-yl)-[1,1′-biphenyl]-3-yl)-3-(((((S)-5-oxopyrrolidin-2-yl)methyl)amino)methyl)-4H-pyrido[1,2-a]pyrimidin-4-one;
2-chloro-8-(2,2′-dichloro-3′-(6-methoxy-5-(((((R)-5-oxopyrrolidin-2-yl)methyl)amino)methyl)pyridin-2-yl)-[1,1′-biphenyl]-3-yl)-3-(((((S)-5-oxopyrrolidin-2-yl)methyl)amino)methyl)-4H-pyrido[1,2-a]pyrimidin-4-one;
2-chloro-8-(2,2′-dichloro-3′-(6-methoxy-5-(((((S)-5-oxopyrrolidin-2-yl)methyl)amino)methyl)pyridin-2-yl)-[1,1′-biphenyl]-3-yl)-3-(((((R)-5-oxopyrrolidin-2-yl)methyl)amino)methyl)-4H-pyrido[1,2-a]pyrimidin-4-one;
2-chloro-8-(2,2′-dichloro-3′-(6-methoxy-5-(((((R)-5-oxopyrrolidin-2-yl)methyl)amino)methyl)pyridin-2-yl)-[1,1′-biphenyl]-3-yl)-3-(((((R)-5-oxopyrrolidin-2-yl)methyl)amino)methyl)-4H-pyrido[1,2-a]pyrimidin-4-one;
(S)-5-((((6-(2,2′-dichloro-3′-(((E)-6-methoxy-5-(((((S)-5-oxopyrrolidin-2-yl)methyl)amino)methyl)-2,3-dihydro-1H-inden-1-ylidene)methyl)-[1,1′-biphenyl]-3-yl)-2-methoxypyridin-3-yl)methyl)amino)methyl)pyrrolidin-2-one;
(R)-5-((((6-(2,2′-dichloro-3′-(((E)-6-methoxy-5-(((((S)-5-oxopyrrolidin-2-yl)methyl)amino)methyl)-2,3-dihydro-1H-inden-1-ylidene)methyl)-[1,1′-biphenyl]-3-yl)-2-methoxypyridin-3-yl)methyl)amino)methyl)pyrrolidin-2-one;
(S)-5-((((6-(2,2′-dichloro-3′-(((E)-6-methoxy-5-(((((R)-5-oxopyrrolidin-2-yl)methyl)amino)methyl)-2,3-dihydro-1H-inden-1-ylidene)methyl)-[1,1′-biphenyl]-3-yl)-2-methoxypyridin-3-yl)methyl)amino)methyl)pyrrolidin-2-one;
(R)-5-((((6-(2,2′-dichloro-3′-(((E)-6-methoxy-5-(((((R)-5-oxopyrrolidin-2-yl)methyl)amino)methyl)-2,3-dihydro-1H-inden-1-ylidene)methyl)-[1,1′-biphenyl]-3-yl)-2-methoxypyridin-3-yl)methyl)amino)methyl)pyrrolidin-2-one;
(S)-5-((((6-(2,2′-dichloro-3′-(((Z)-6-methoxy-5-(((((S)-5-oxopyrrolidin-2-yl)methyl)amino)methyl)-2,3-dihydro-1H-inden-1-ylidene)methyl)-[1,1′-biphenyl]-3-yl)-2-methoxypyridin-3-yl)methyl)amino)methyl)pyrrolidin-2-one;
(R)-5-((((6-(2,2′-dichloro-3′-(((Z)-6-methoxy-5-(((((S)-5-oxopyrrolidin-2-yl)methyl)amino)methyl)-2,3-dihydro-1H-inden-1-ylidene)methyl)-[1,1′-biphenyl]-3-yl)-2-methoxypyridin-3-yl)methyl)amino)methyl)pyrrolidin-2-one;
(S)-5-((((6-(2,2′-dichloro-3′-(((Z)-6-methoxy-5-(((((R)-5-oxopyrrolidin-2-yl)methyl)amino)methyl)-2,3-dihydro-1H-inden-1-ylidene)methyl)-[1,1′-biphenyl]-3-yl)-2-methoxypyridin-3-yl)methyl)amino)methyl)pyrrolidin-2-one;
(R)-5-((((6-(2,2′-dichloro-3′-(((Z)-6-methoxy-5-(((((R)-5-oxopyrrolidin-2-yl)methyl)amino)methyl)-2,3-dihydro-1H-inden-1-ylidene)methyl)-[1,1′-biphenyl]-3-yl)-2-methoxypyridin-3-yl)methyl)amino)methyl)pyrrolidin-2-one;
8-(2,2′-dichloro-3′-(6-methoxy-5-(((((S)-5-oxopyrrolidin-2-yl)methyl)amino)methyl)pyridin-2-yl)-[1,1′-biphenyl]-3-yl)-3-((((1s,3s)-3-hydroxy-3-methylcyclobutyl)amino)methyl)-2-methyl-4H-pyrido[1,2-a]pyrimidin-4-one;
8-(2,2′-dichloro-3′-(6-methoxy-5-(((((S)-5-oxopyrrolidin-2-yl)methyl)amino)methyl)pyridin-2-yl)-[1,1′-biphenyl]-3-yl)-3-((((1r,3r)-3-hydroxy-3-methylcyclobutyl)amino)methyl)-2-methyl-4H-pyrido[1,2-a]pyrimidin-4-one;
8-(2,2′-dichloro-3′-(6-methoxy-5-(((((R)-5-oxopyrrolidin-2-yl)methyl)amino)methyl)pyridin-2-yl)-[1,1′-biphenyl]-3-yl)-3-((((1s,3s)-3-hydroxy-3-methylcyclobutyl)amino)methyl)-2-methyl-4H-pyrido[1,2-a]pyrimidin-4-one;
8-(2,2′-dichloro-3′-(6-methoxy-5-(((((R)-5-oxopyrrolidin-2-yl)methyl)amino)methyl)pyridin-2-yl)-[1,1′-biphenyl]-3-yl)-3-((((1r,3r)-3-hydroxy-3-methylcyclobutyl)amino)methyl)-2-methyl-4H-pyrido[1,2-a]pyrimidin-4-one;
8-(2,2′-dichloro-3′-(5-((((1s,3s)-3-hydroxy-3-methylcyclobutyl)amino)methyl)-6-methoxypyridin-2-yl)-[1,1′-biphenyl]-3-yl)-3-((((1s,3s)-3-hydroxy-3-methylcyclobutyl)amino)methyl)-2-methyl-4H-pyrido[1,2-a]pyrimidin-4-one;
8-(2,2′-dichloro-3′-(5-((((1r,3r)-3-hydroxy-3-methylcyclobutyl)amino)methyl)-6-methoxypyridin-2-yl)-[1,1′-biphenyl]-3-yl)-3-((((1s,3s)-3-hydroxy-3-methylcyclobutyl)amino)methyl)-2-methyl-4H-pyrido[1,2-a]pyrimidin-4-one;
8-(2,2′-dichloro-3′-(5-((((1s,3s)-3-hydroxy-3-methylcyclobutyl)amino)methyl)-6-methoxypyridin-2-yl)-[1,1′-biphenyl]-3-yl)-3-((((1r,3r)-3-hydroxy-3-methylcyclobutyl)amino)methyl)-2-methyl-4H-pyrido[1,2-a]pyrimidin-4-one;
8-(2,2′-dichloro-3′-(5-((((1r,3r)-3-hydroxy-3-methylcyclobutyl)amino)methyl)-6-methoxypyridin-2-yl)-[1,1′-biphenyl]-3-yl)-3-((((1r,3r)-3-hydroxy-3-methylcyclobutyl)amino)methyl)-2-methyl-4H-pyrido[1,2-a]pyrimidin-4-one;
methyl (S)-4-(((6-(2,2′-dichloro-3′-(3-((((S)-2-hydroxy-4-methoxy-4-oxobutyl)amino)methyl)-4-oxo-4H-pyrido[1,2-a]pyrimidin-8-yl)-[1,1′-biphenyl]-3-yl)-2-methoxypyridin-3-yl)methyl)amino)-3-hydroxybutanoate;
methyl (R)-4-(((6-(2,2′-dichloro-3′-(3-((((S)-2-hydroxy-4-methoxy-4-oxobutyl)amino)methyl)-4-oxo-4H-pyrido[1,2-a]pyrimidin-8-yl)-[1,1′-biphenyl]-3-yl)-2-methoxypyridin-3-yl)methyl)amino)-3-hydroxybutanoate;
methyl (S)-4-(((6-(2,2′-dichloro-3′-(3-((((R)-2-hydroxy-4-methoxy-4-oxobutyl)amino)methyl)-4-oxo-4H-pyrido[1,2-a]pyrimidin-8-yl)-[1,1′-biphenyl]-3-yl)-2-methoxypyridin-3-yl)methyl)amino)-3-hydroxybutanoate;
methyl (R)-4-(((6-(2,2′-dichloro-3′-(3-((((R)-2-hydroxy-4-methoxy-4-oxobutyl)amino)methyl)-4-oxo-4H-pyrido[1,2-a]pyrimidin-8-yl)-[1,1′-biphenyl]-3-yl)-2-methoxypyridin-3-yl)methyl)amino)-3-hydroxybutanoate;
(S)-5-((((6-(2,2′-dichloro-3′-(4-fluoro-6-methoxy-5-(((((S)-5-oxopyrrolidin-2-yl)methyl)amino)methyl)pyridin-2-yl)-[1,1′-biphenyl]-3-yl)-2-methoxypyridin-3-yl)methyl)amino)methyl)pyrrolidin-2-one;
(R)-5-((((6-(2,2′-dichloro-3′-(4-fluoro-6-methoxy-5-(((((S)-5-oxopyrrolidin-2-yl)methyl)amino)methyl)pyridin-2-yl)-[1,1′-biphenyl]-3-yl)-2-methoxypyridin-3-yl)methyl)amino)methyl)pyrrolidin-2-one;
(S)-5-((((6-(2,2′-dichloro-3′-(4-fluoro-6-methoxy-5-(((((R)-5-oxopyrrolidin-2-yl)methyl)amino)methyl)pyridin-2-yl)-[1,1′-biphenyl]-3-yl)-2-methoxypyridin-3-yl)methyl)amino)methyl)pyrrolidin-2-one;
(R)-5-((((6-(2,2′-dichloro-3′-(4-fluoro-6-methoxy-5-(((((R)-5-oxopyrrolidin-2-yl)methyl)amino)methyl)pyridin-2-yl)-[1,1′-biphenyl]-3-yl)-2-methoxypyridin-3-yl)methyl)amino)methyl)pyrrolidin-2-one;
(S)-8-(2,2′-dichloro-3′-(6-methoxy-5-((((5-oxopyrrolidin-2-yl)methyl)amino)methyl)pyridin-2-yl)-[1,1′-biphenyl]-3-yl)-3-((((1-hydroxycyclobutyl)methyl)amino)methyl)-4H-pyrido[1,2-a]pyrimidin-4-one;
(R)-8-(2,2′-dichloro-3′-(6-methoxy-5-((((5-oxopyrrolidin-2-yl)methyl)amino)methyl)pyridin-2-yl)-[1,1′-biphenyl]-3-yl)-3-((((1-hydroxycyclobutyl)methyl)amino)methyl)-4H-pyrido[1,2-a]pyrimidin-4-one;
(S)-8-(2,2′-dichloro-3′-(6-methoxy-5-((((5-oxopyrrolidin-2-yl)methyl)amino)methyl)pyridin-2-yl)-[1,1′-biphenyl]-3-yl)-3-((((1-hydroxycyclopropyl)methyl)amino)methyl)-4H-pyrido[1,2-a]pyrimidin-4-one;
(R)-8-(2,2′-dichloro-3′-(6-methoxy-5-((((5-oxopyrrolidin-2-yl)methyl)amino)methyl)pyridin-2-yl)-[1,1′-biphenyl]-3-yl)-3-((((1-hydroxycyclopropyl)methyl)amino)methyl)-4H-pyrido[1,2-a]pyrimidin-4-one;
(5S)-5-((((6-(2,2′-dichloro-3′-((R)-5-((((S)-5-oxopyrrolidin-2-yl)methyl)amino)-5,6,7,8-tetrahydroquinolin-2-yl)-[1,1′-biphenyl]-3-yl)-2-methoxypyridin-3-yl)methyl)amino)methyl)pyrrolidin-2-one;
(5R)-5-((((6-(2,2′-dichloro-3′-((R)-5-((((S)-5-oxopyrrolidin-2-yl)methyl)amino)-5,6,7,8-tetrahydroquinolin-2-yl)-[1,1′-biphenyl]-3-yl)-2-methoxypyridin-3-yl)methyl)amino)methyl)pyrrolidin-2-one;
(5S)-5-((((6-(2,2′-dichloro-3′-((R)-5-((((R)-5-oxopyrrolidin-2-yl)methyl)amino)-5,6,7,8-tetrahydroquinolin-2-yl)-[1,1′-biphenyl]-3-yl)-2-methoxypyridin-3-yl)methyl)amino)methyl)pyrrolidin-2-one;
(5R)-5-((((6-(2,2′-dichloro-3′-((R)-5-((((R)-5-oxopyrrolidin-2-yl)methyl)amino)-5,6,7,8-tetrahydroquinolin-2-yl)-[1,1′-biphenyl]-3-yl)-2-methoxypyridin-3-yl)methyl)amino)methyl)pyrrolidin-2-one;
(5S)-5-((((6-(2,2′-dichloro-3′-((S)-5-((((S)-5-oxopyrrolidin-2-yl)methyl)amino)-5,6,7,8-tetrahydroquinolin-2-yl)-[1,1′-biphenyl]-3-yl)-2-methoxypyridin-3-yl)methyl)amino)methyl)pyrrolidin-2-one;
(5R)-5-((((6-(2,2′-dichloro-3′-((S)-5-((((S)-5-oxopyrrolidin-2-yl)methyl)amino)-5,6,7,8-tetrahydroquinolin-2-yl)-[1,1′-biphenyl]-3-yl)-2-methoxypyridin-3-yl)methyl)amino)methyl)pyrrolidin-2-one;
(5S)-5-((((6-(2,2′-dichloro-3′-((S)-5-((((R)-5-oxopyrrolidin-2-yl)methyl)amino)-5,6,7,8-tetrahydroquinolin-2-yl)-[1,1′-biphenyl]-3-yl)-2-methoxypyridin-3-yl)methyl)amino)methyl)pyrrolidin-2-one;
(5R)-5-((((6-(2,2′-dichloro-3′-((S)-5-((((R)-5-oxopyrrolidin-2-yl)methyl)amino)-5,6,7,8-tetrahydroquinolin-2-yl)-[1,1′-biphenyl]-3-yl)-2-methoxypyridin-3-yl)methyl)amino)methyl)pyrrolidin-2-one;
(5S,5'S)-5,5′-(((((2,2′-dichloro-[1,1′-biphenyl]-3,3′-diyl)bis(4-fluoro-2-methoxypyridine-6,3-diyl))bis(methylene))bis(azanediyl))bis(methylene))bis(pyrrolidin-2-one);
(5R,5'S)-5,5′-(((((2,2′-dichloro-[1,1′-biphenyl]-3,3′-diyl)bis(4-fluoro-2-methoxypyridine-6,3-diyl))bis(methylene))bis(azanediyl))bis(methylene))bis(pyrrolidin-2-one);
(5S,5′R)-5,5′-(((((2,2′-dichloro-[1,1′-biphenyl]-3,3′-diyl)bis(4-fluoro-2-methoxypyridine-6,3-diyl))bis(methylene))bis(azanediyl))bis(methylene))bis(pyrrolidin-2-one);
(5R,5′R)-5,5′-(((((2,2′-dichloro-[1,1′-biphenyl]-3,3′-diyl)bis(4-fluoro-2-methoxypyridine-6,3-diyl))bis(methylene))bis(azanediyl))bis(methylene))bis(pyrrolidin-2-one);
(S)-8-(4″-((1-aminocyclopropyl)methyl)-2,2′-dichloro-[1,1′:3′,1″-terphenyl]-3-yl)-3-((((5-oxopyrrolidin-2-yl)methyl)amino)methyl)-4H-pyrido[1,2-a]pyrimidin-4-one;
(R)-8-(4″-((1-aminocyclopropyl)methyl)-2,2′-dichloro-[1,1′:3′,1″-terphenyl]-3-yl)-3-((((5-oxopyrrolidin-2-yl)methyl)amino)methyl)-4H-pyrido[1,2-a]pyrimidin-4-one;
(S)-4-(((8-(2,2′-dichloro-3′-(6-methoxy-5-(((((S)-5-oxopyrrolidin-2-yl)methyl)amino)methyl)pyridin-2-yl)-[1,1′-biphenyl]-3-yl)-4-oxo-4H-pyrido[1,2-a]pyrimidin-3-yl)methyl)amino)-3-hydroxybutanamide;
(R)-4-(((8-(2,2′-dichloro-3′-(6-methoxy-5-(((((S)-5-oxopyrrolidin-2-yl)methyl)amino)methyl)pyridin-2-yl)-[1,1′-biphenyl]-3-yl)-4-oxo-4H-pyrido[1,2-a]pyrimidin-3-yl)methyl)amino)-3-hydroxybutanamide;
(S)-4-(((8-(2,2′-dichloro-3′-(6-methoxy-5-(((((R)-5-oxopyrrolidin-2-yl)methyl)amino)methyl)pyridin-2-yl)-[1,1′-biphenyl]-3-yl)-4-oxo-4H-pyrido[1,2-a]pyrimidin-3-yl)methyl)amino)-3-hydroxybutanamide;
(R)-4-(((8-(2,2′-dichloro-3′-(6-methoxy-5-(((((R)-5-oxopyrrolidin-2-yl)methyl)amino)methyl)pyridin-2-yl)-[1,1′-biphenyl]-3-yl)-4-oxo-4H-pyrido[1,2-a]pyrimidin-3-yl)methyl)amino)-3-hydroxybutanamide;
(S)-6-(2,2′-dichloro-3′-(6-methoxy-5-((((5-oxopyrrolidin-2-yl)methyl)amino)methyl)pyridin-2-yl)-[1,1′-biphenyl]-3-yl)-2-(2-hydroxyethyl)-[1,2,4]triazolo[4,3-a]pyridin-3(2H)-one;
(R)-6-(2,2′-dichloro-3′-(6-methoxy-5-((((5-oxopyrrolidin-2-yl)methyl)amino)methyl)pyridin-2-yl)-[1,1′-biphenyl]-3-yl)-2-(2-hydroxyethyl)-[1,2,4]triazolo[4,3-a]pyridin-3(2H)-one;
(S)-5-((((6-(2,2′-dichloro-3′-(5,6,7,8-tetrahydro-[1,2,4]triazolo[4,3-a]pyrazin-3-yl)-[1,1′-biphenyl]-3-yl)-2-methoxypyridin-3-yl)methyl)amino)methyl)pyrrolidin-2-one;
(R)-5-((((6-(2,2′-dichloro-3′-(5,6,7,8-tetrahydro-[1,2,4]triazolo[4,3-a]pyrazin-3-yl)-[1,1′-biphenyl]-3-yl)-2-methoxypyridin-3-yl)methyl)amino)methyl)pyrrolidin-2-one;
(S)-5-((((6-(2,2′-dichloro-3′-(2-(4-(2-hydroxyethyl)piperazin-1-yl)imidazo[2,1-b][1,3,4]thiadiazol-5-yl)-[1,1′-biphenyl]-3-yl)-2-methoxypyridin-3-yl)methyl)amino)methyl)pyrrolidin-2-one;
(R)-5-((((6-(2,2′-dichloro-3′-(2-(4-(2-hydroxyethyl)piperazin-1-yl)imidazo[2,1-b][1,3,4]thiadiazol-5-yl)-[1,1′-biphenyl]-3-yl)-2-methoxypyridin-3-yl)methyl)amino)methyl)pyrrolidin-2-one;
(S)-5-((((6-(2,2′-dichloro-3′-(7-(2-hydroxyethyl)-5,6,7,8-tetrahydro-[1,2,4]triazolo[4,3-a]pyrazin-3-yl)-[1,1′-biphenyl]-3-yl)-2-methoxypyridin-3-yl)methyl)amino)methyl)pyrrolidin-2-one;
(R)-5-((((6-(2,2′-dichloro-3′-(7-(2-hydroxyethyl)-5,6,7,8-tetrahydro-[1,2,4]triazolo[4,3-a]pyrazin-3-yl)-[1,1′-biphenyl]-3-yl)-2-methoxypyridin-3-yl)methyl)amino)methyl)pyrrolidin-2-one;
(S)-8-(2,2′-dichloro-3′-(6-methoxy-5-((((5-oxopyrrolidin-2-yl)methyl)amino)methyl)pyridin-2-yl)-[1,1′-biphenyl]-3-yl)-3-((((3-hydroxyoxetan-3-yl)methyl)amino)methyl)-4H-pyrido[1,2-a]pyrimidin-4-one;
(R)-8-(2,2′-dichloro-3′-(6-methoxy-5-((((5-oxopyrrolidin-2-yl)methyl)amino)methyl)pyridin-2-yl)-[1,1′-biphenyl]-3-yl)-3-((((3-hydroxyoxetan-3-yl)methyl)amino)methyl)-4H-pyrido[1,2-a]pyrimidin-4-one;
(S)-8-(2,2′-dichloro-3′-(6-methoxy-5-((((5-oxopyrrolidin-2-yl)methyl)amino)methyl)pyridin-2-yl)-[1,1′-biphenyl]-3-yl)-3-((((3-(hydroxymethyl)oxetan-3-yl)methyl)amino)methyl)-4H-pyrido[1,2-a]pyrimidin-4-one;
(R)-8-(2,2′-dichloro-3′-(6-methoxy-5-((((5-oxopyrrolidin-2-yl)methyl)amino)methyl)pyridin-2-yl)-[1,1′-biphenyl]-3-yl)-3-((((3-(hydroxymethyl)oxetan-3-yl)methyl)amino)methyl)-4H-pyrido[1,2-a]pyrimidin-4-one;
(S)-8-(2,2′-dichloro-3′-(6-methoxy-5-((((5-oxopyrrolidin-2-yl)methyl)amino)methyl)pyridin-2-yl)-[1,1′-biphenyl]-3-yl)-3-((((3-methyloxetan-3-yl)methyl)amino)methyl)-4H-pyrido[1,2-a]pyrimidin-4-one;
(R)-8-(2,2′-dichloro-3′-(6-methoxy-5-((((5-oxopyrrolidin-2-yl)methyl)amino)methyl)pyridin-2-yl)-[1,1′-biphenyl]-3-yl)-3-((((3-methyloxetan-3-yl)methyl)amino)methyl)-4H-pyrido[1,2-a]pyrimidin-4-one;
(S)-5-((((6-(2,2′-dichloro-3′-(2-(((((S)-5-oxopyrrolidin-2-yl)methyl)amino)methyl)benzo[d]oxazol-5-yl)-[1,1′-biphenyl]-3-yl)-2-methoxypyridin-3-yl)methyl)amino)methyl)pyrrolidin-2-one;
(R)-5-((((6-(2,2′-dichloro-3′-(2-(((((S)-5-oxopyrrolidin-2-yl)methyl)amino)methyl)benzo[d]oxazol-5-yl)-[1,1′-biphenyl]-3-yl)-2-methoxypyridin-3-yl)methyl)amino)methyl)pyrrolidin-2-one;
(S)-5-((((6-(2,2′-dichloro-3′-(2-(((((R)-5-oxopyrrolidin-2-yl)methyl)amino)methyl)benzo[d]oxazol-5-yl)-[1,1′-biphenyl]-3-yl)-2-methoxypyridin-3-yl)methyl)amino)methyl)pyrrolidin-2-one;
(R)-5-((((6-(2,2′-dichloro-3′-(2-(((((R)-5-oxopyrrolidin-2-yl)methyl)amino)methyl)benzo[d]oxazol-5-yl)-[1,1′-biphenyl]-3-yl)-2-methoxypyridin-3-yl)methyl)amino)methyl)pyrrolidin-2-one;
(S)-5-((((6-(2,2′-dichloro-3′-(2-(((((S)-5-oxopyrrolidin-2-yl)methyl)amino)methyl)benzo[d]oxazol-6-yl)-[1,1′-biphenyl]-3-yl)-2-methoxypyridin-3-yl)methyl)amino)methyl)pyrrolidin-2-one;
(R)-5-((((6-(2,2′-dichloro-3′-(2-(((((S)-5-oxopyrrolidin-2-yl)methyl)amino)methyl)benzo[d]oxazol-6-yl)-[1,1′-biphenyl]-3-yl)-2-methoxypyridin-3-yl)methyl)amino)methyl)pyrrolidin-2-one;
(S)-5-((((6-(2,2′-dichloro-3′-(2-(((((R)-5-oxopyrrolidin-2-yl)methyl)amino)methyl)benzo[d]oxazol-6-yl)-[1,1′-biphenyl]-3-yl)-2-methoxypyridin-3-yl)methyl)amino)methyl)pyrrolidin-2-one;
(R)-5-((((6-(2,2′-dichloro-3′-(2-(((((R)-5-oxopyrrolidin-2-yl)methyl)amino)methyl)benzo[d]oxazol-6-yl)-[1,1′-biphenyl]-3-yl)-2-methoxypyridin-3-yl)methyl)amino)methyl)pyrrolidin-2-one;
(S)-8-(2,2′-dichloro-3′-(6-methoxy-5-(((((S)-5-oxopyrrolidin-2-yl)methyl)amino)methyl)pyridin-2-yl)-[1,1′-biphenyl]-3-yl)-3-(((5-oxo-6-azaspiro[3.4]octan-2-yl)amino)methyl)-4H-pyrido[1,2-a]pyrimidin-4-one;
(R)-8-(2,2′-dichloro-3′-(6-methoxy-5-(((((S)-5-oxopyrrolidin-2-yl)methyl)amino)methyl)pyridin-2-yl)-[1,1′-biphenyl]-3-yl)-3-(((5-oxo-6-azaspiro[3.4]octan-2-yl)amino)methyl)-4H-pyrido[1,2-a]pyrimidin-4-one;
(S)-8-(2,2′-dichloro-3′-(6-methoxy-5-(((((R)-5-oxopyrrolidin-2-yl)methyl)amino)methyl)pyridin-2-yl)-[1,1′-biphenyl]-3-yl)-3-(((5-oxo-6-azaspiro[3.4]octan-2-yl)amino)methyl)-4H-pyrido[1,2-a]pyrimidin-4-one;
(R)-8-(2,2′-dichloro-3′-(6-methoxy-5-(((((R)-5-oxopyrrolidin-2-yl)methyl)amino)methyl)pyridin-2-yl)-[1,1′-biphenyl]-3-yl)-3-(((5-oxo-6-azaspiro[3.4]octan-2-yl)amino)methyl)-4H-pyrido[1,2-a]pyrimidin-4-one;
8-(2,2′-dichloro-3′-(6-methoxy-5-(((((S)-5-oxopyrrolidin-2-yl)methyl)amino)methyl)pyridin-2-yl)-[1,1′-biphenyl]-3-yl)-3-((((3-oxo-2-azabicyclo[2.2.1]heptan-1-yl)methyl)amino)methyl)-4H-pyrido[1,2-a]pyrimidin-4-one;
8-(2,2′-dichloro-3′-(6-methoxy-5-(((((R)-5-oxopyrrolidin-2-yl)methyl)amino)methyl)pyridin-2-yl)-[1,1′-biphenyl]-3-yl)-3-((((3-oxo-2-azabicyclo[2.2.1]heptan-1-yl)methyl)amino)methyl)-4H-pyrido[1,2-a]pyrimidin-4-one;
8-(2,2′-dichloro-3′-(6-methoxy-5-(((((S)-5-oxopyrrolidin-2-yl)methyl)amino)methyl)pyridin-2-yl)-[1,1′-biphenyl]-3-yl)-3-(((((S)-4,4-difluoro-5-oxopyrrolidin-2-yl)methyl)amino)methyl)-4H-pyrido[1,2-a]pyrimidin-4-one;
8-(2,2′-dichloro-3′-(6-methoxy-5-(((((S)-5-oxopyrrolidin-2-yl)methyl)amino)methyl)pyridin-2-yl)-[1,1′-biphenyl]-3-yl)-3-(((((R)-4,4-difluoro-5-oxopyrrolidin-2-yl)methyl)amino)methyl)-4H-pyrido[1,2-a]pyrimidin-4-one;
8-(2,2′-dichloro-3′-(6-methoxy-5-(((((R)-5-oxopyrrolidin-2-yl)methyl)amino)methyl)pyridin-2-yl)-[1,1′-biphenyl]-3-yl)-3-(((((S)-4,4-difluoro-5-oxopyrrolidin-2-yl)methyl)amino)methyl)-4H-pyrido[1,2-a]pyrimidin-4-one;
8-(2,2′-dichloro-3′-(6-methoxy-5-(((((R)-5-oxopyrrolidin-2-yl)methyl)amino)methyl)pyridin-2-yl)-[1,1′-biphenyl]-3-yl)-3-(((((R)-4,4-difluoro-5-oxopyrrolidin-2-yl)methyl)amino)methyl)-4H-pyrido[1,2-a]pyrimidin-4-one;
8-(2,2′-dichloro-3′-(6-methoxy-5-(((((S)-5-oxopyrrolidin-2-yl)methyl)amino)methyl)pyridin-2-yl)-[1,1′-biphenyl]-3-yl)-3-((((1s,3s)-3-hydroxy-3-methylcyclobutyl)amino)methyl)-6-methyl-4H-pyrido[1,2-a]pyrimidin-4-one;
8-(2,2′-dichloro-3′-(6-methoxy-5-(((((R)-5-oxopyrrolidin-2-yl)methyl)amino)methyl)pyridin-2-yl)-[1,1′-biphenyl]-3-yl)-3-((((1s,3s)-3-hydroxy-3-methylcyclobutyl)amino)methyl)-6-methyl-4H-pyrido[1,2-a]pyrimidin-4-one;
8-(2,2′-dichloro-3′-(6-methoxy-5-(((((S)-5-oxopyrrolidin-2-yl)methyl)amino)methyl)pyridin-2-yl)-[1,1′-biphenyl]-3-yl)-3-((((1r,3r)-3-hydroxy-3-methylcyclobutyl)amino)methyl)-6-methyl-4H-pyrido[1,2-a]pyrimidin-4-one;
8-(2,2′-dichloro-3′-(6-methoxy-5-(((((R)-5-oxopyrrolidin-2-yl)methyl)amino)methyl)pyridin-2-yl)-[1,1′-biphenyl]-3-yl)-3-((((1r,3r)-3-hydroxy-3-methylcyclobutyl)amino)methyl)-6-methyl-4H-pyrido[1,2-a]pyrimidin-4-one;
(S)-1-((8-(2,2′-dichloro-3′-(6-methoxy-5-((((5-oxopyrrolidin-2-yl)methyl)amino)methyl)pyridin-2-yl)-[1,1′-biphenyl]-3-yl)-4-oxo-4H-pyrido[1,2-a]pyrimidin-3-yl)methyl)-N-methylazetidine-3-carboxamide;
(R)-1-((8-(2,2′-dichloro-3′-(6-methoxy-5-((((5-oxopyrrolidin-2-yl)methyl)amino)methyl)pyridin-2-yl)-[1,1′-biphenyl]-3-yl)-4-oxo-4H-pyrido[1,2-a]pyrimidin-3-yl)methyl)-N-methylazetidine-3-carboxamide;
(S)-3-(((1-acetylpiperidin-4-yl)amino)methyl)-8-(2,2′-dichloro-3′-(6-methoxy-5-((((5-oxopyrrolidin-2-yl)methyl)amino)methyl)pyridin-2-yl)-[1,1′-biphenyl]-3-yl)-4H-pyrido[1,2-a]pyrimidin-4-one;
(R)-3-(((1-acetylpiperidin-4-yl)amino)methyl)-8-(2,2′-dichloro-3′-(6-methoxy-5-((((5-oxopyrrolidin-2-yl)methyl)amino)methyl)pyridin-2-yl)-[1,1′-biphenyl]-3-yl)-4H-pyrido[1,2-a]pyrimidin-4-one;
(S)-1-((8-(2,2′-dichloro-3′-(6-methoxy-5-(((((S)-5-oxopyrrolidin-2-yl)methyl)amino)methyl)pyridin-2-yl)-[1,1′-biphenyl]-3-yl)-4-oxo-4H-pyrido[1,2-a]pyrimidin-3-yl)methyl)-N-methylpyrrolidine-3-carboxamide;
(R)-1-((8-(2,2′-dichloro-3′-(6-methoxy-5-(((((S)-5-oxopyrrolidin-2-yl)methyl)amino)methyl)pyridin-2-yl)-[1,1′-biphenyl]-3-yl)-4-oxo-4H-pyrido[1,2-a]pyrimidin-3-yl)methyl)-N-methylpyrrolidine-3-carboxamide;
(S)-1-((8-(2,2′-dichloro-3′-(6-methoxy-5-(((((R)-5-oxopyrrolidin-2-yl)methyl)amino)methyl)pyridin-2-yl)-[1,1′-biphenyl]-3-yl)-4-oxo-4H-pyrido[1,2-a]pyrimidin-3-yl)methyl)-N-methylpyrrolidine-3-carboxamide;
(R)-1-((8-(2,2′-dichloro-3′-(6-methoxy-5-(((((R)-5-oxopyrrolidin-2-yl)methyl)amino)methyl)pyridin-2-yl)-[1,1′-biphenyl]-3-yl)-4-oxo-4H-pyrido[1,2-a]pyrimidin-3-yl)methyl)-N-methylpyrrolidine-3-carboxamide;
(S)-5-((((6-(2,2′-dichloro-3′-(3-(((((S)-5-oxopyrrolidin-2-yl)methyl)amino)methyl)-1,6-naphthyridin-7-yl)-[1,1′-biphenyl]-3-yl)-2-methoxypyridin-3-yl)methyl)amino)methyl)pyrrolidin-2-one;
(R)-5-((((6-(2,2′-dichloro-3′-(3-(((((S)-5-oxopyrrolidin-2-yl)methyl)amino)methyl)-1,6-naphthyridin-7-yl)-[1,1′-biphenyl]-3-yl)-2-methoxypyridin-3-yl)methyl)amino)methyl)pyrrolidin-2-one;
(S)-5-((((6-(2,2′-dichloro-3′-(3-(((((R)-5-oxopyrrolidin-2-yl)methyl)amino)methyl)-1,6-naphthyridin-7-yl)-[1,1′-biphenyl]-3-yl)-2-methoxypyridin-3-yl)methyl)amino)methyl)pyrrolidin-2-one;
(R)-5-((((6-(2,2′-dichloro-3′-(3-(((((R)-5-oxopyrrolidin-2-yl)methyl)amino)methyl)-1,6-naphthyridin-7-yl)-[1,1′-biphenyl]-3-yl)-2-methoxypyridin-3-yl)methyl)amino)methyl)pyrrolidin-2-one;
1-((8-(2,2′-dichloro-3′-(6-methoxy-5-(((((S)-5-oxopyrrolidin-2-yl)methyl)amino)methyl)pyridin-2-yl)-[1,1′-biphenyl]-3-yl)-4-oxo-4H-pyrido[1,2-a]pyrimidin-3-yl)methyl)azetidine-(2S)-carboxamide;
1-((8-(2,2′-dichloro-3′-(6-methoxy-5-(((((R)-5-oxopyrrolidin-2-yl)methyl)amino)methyl)pyridin-2-yl)-[1,1′-biphenyl]-3-yl)-4-oxo-4H-pyrido[1,2-a]pyrimidin-3-yl)methyl)azetidine-(2S)-carboxamide;
1-((8-(2,2′-dichloro-3′-(6-methoxy-5-(((((S)-5-oxopyrrolidin-2-yl)methyl)amino)methyl)pyridin-2-yl)-[1,1′-biphenyl]-3-yl)-4-oxo-4H-pyrido[1,2-a]pyrimidin-3-yl)methyl)azetidine-(2R)-carboxamide;
1-((8-(2,2′-dichloro-3′-(6-methoxy-5-(((((R)-5-oxopyrrolidin-2-yl)methyl)amino)methyl)pyridin-2-yl)-[1,1′-biphenyl]-3-yl)-4-oxo-4H-pyrido[1,2-a]pyrimidin-3-yl)methyl)azetidine-(2R)-carboxamide;
(S)-2-(((8-(2,2′-dichloro-3′-(6-methoxy-5-(((((S)-5-oxopyrrolidin-2-yl)methyl)amino)methyl)pyridin-2-yl)-[1,1′-biphenyl]-3-yl)-4-oxo-4H-pyrido[1,2-a]pyrimidin-3-yl)methyl)amino)propenamide;
(R)-2-(((8-(2,2′-dichloro-3′-(6-methoxy-5-(((((S)-5-oxopyrrolidin-2-yl)methyl)amino)methyl)pyridin-2-yl)-[1,1′-biphenyl]-3-yl)-4-oxo-4H-pyrido[1,2-a]pyrimidin-3-yl)methyl)amino)propenamide;
(S)-2-(((8-(2,2′-dichloro-3′-(6-methoxy-5-(((((R)-5-oxopyrrolidin-2-yl)methyl)amino)methyl)pyridin-2-yl)-[1,1′-biphenyl]-3-yl)-4-oxo-4H-pyrido[1,2-a]pyrimidin-3-yl)methyl)amino)propenamide;
(R)-2-(((8-(2,2′-dichloro-3′-(6-methoxy-5-(((((R)-5-oxopyrrolidin-2-yl)methyl)amino)methyl)pyridin-2-yl)-[1,1′-biphenyl]-3-yl)-4-oxo-4H-pyrido[1,2-a]pyrimidin-3-yl)methyl)amino)propenamide;
(S)-8-(2,2′-dichloro-3′-(2,3,4,5-tetrahydropyrido[3,2-f][1,4]oxazepin-8-yl)-[1,1′-biphenyl]-3-yl)-3-((((5-oxopyrrolidin-2-yl)methyl)amino)methyl)-4H-pyrido[1,2-a]pyrimidin-4-one;
(R)-8-(2,2′-dichloro-3′-(2,3,4,5-tetrahydropyrido[3,2-f][1,4]oxazepin-8-yl)-[1,1′-biphenyl]-3-yl)-3-((((5-oxopyrrolidin-2-yl)methyl)amino)methyl)-4H-pyrido[1,2-a]pyrimidin-4-one;
8-(2,2′-dichloro-3′-(6-methoxy-5-(((((S)-5-oxopyrrolidin-2-yl)methyl)amino)methyl)pyridin-2-yl)-[1,1′-biphenyl]-3-yl)-3-((((S)-2-hydroxypropyl)amino)methyl)-2-methoxy-4H-pyrido[1,2-a]pyrimidin-4-one;
8-(2,2′-dichloro-3′-(6-methoxy-5-(((((R)-5-oxopyrrolidin-2-yl)methyl)amino)methyl)pyridin-2-yl)-[1,1′-biphenyl]-3-yl)-3-((((S)-2-hydroxypropyl)amino)methyl)-2-methoxy-4H-pyrido[1,2-a]pyrimidin-4-one;
8-(2,2′-dichloro-3′-(6-methoxy-5-(((((S)-5-oxopyrrolidin-2-yl)methyl)amino)methyl)pyridin-2-yl)-[1,1′-biphenyl]-3-yl)-3-((((R)-2-hydroxypropyl)amino)methyl)-2-methoxy-4H-pyrido[1,2-a]pyrimidin-4-one;
8-(2,2′-dichloro-3′-(6-methoxy-5-(((((R)-5-oxopyrrolidin-2-yl)methyl)amino)methyl)pyridin-2-yl)-[1,1′-biphenyl]-3-yl)-3-((((R)-2-hydroxypropyl)amino)methyl)-2-methoxy-4H-pyrido[1,2-a]pyrimidin-4-one;
(S)-2-(((8-(2,2′-dichloro-3′-(6-methoxy-5-(((((S)-5-oxopyrrolidin-2-yl)methyl)amino)methyl)pyridin-2-yl)-[1,1′-biphenyl]-3-yl)-4-oxo-4H-pyrido[1,2-a]pyrimidin-3-yl)methyl)(methyl)amino)-N-methylpropanamide;
(R)-2-(((8-(2,2′-dichloro-3′-(6-methoxy-5-(((((S)-5-oxopyrrolidin-2-yl)methyl)amino)methyl)pyridin-2-yl)-[1,1′-biphenyl]-3-yl)-4-oxo-4H-pyrido[1,2-a]pyrimidin-3-yl)methyl)(methyl)amino)-N-methylpropanamide;
(S)-2-(((8-(2,2′-dichloro-3′-(6-methoxy-5-(((((R)-5-oxopyrrolidin-2-yl)methyl)amino)methyl)pyridin-2-yl)-[1,1′-biphenyl]-3-yl)-4-oxo-4H-pyrido[1,2-a]pyrimidin-3-yl)methyl)(methyl)amino)-N-methylpropanamide;
(R)-2-(((8-(2,2′-dichloro-3′-(6-methoxy-5-(((((R)-5-oxopyrrolidin-2-yl)methyl)amino)methyl)pyridin-2-yl)-[1,1′-biphenyl]-3-yl)-4-oxo-4H-pyrido[1,2-a]pyrimidin-3-yl)methyl)(methyl)amino)-N-methylpropanamide;
(S)-8-(2,2′-dichloro-3′-(6-methoxy-5-((((5-oxopyrrolidin-2-yl)methyl)amino)methyl)pyridin-2-yl)-[1,1′-biphenyl]-3-yl)-3-(((2-(3-oxopiperazin-1-yl)ethyl)amino)methyl)-4H-pyrido[1,2-a]pyrimidin-4-one;
(R)-8-(2,2′-dichloro-3′-(6-methoxy-5-((((5-oxopyrrolidin-2-yl)methyl)amino)methyl)pyridin-2-yl)-[1,1′-biphenyl]-3-yl)-3-(((2-(3-oxopiperazin-1-yl)ethyl)amino)methyl)-4H-pyrido[1,2-a]pyrimidin-4-one;
methyl (S)-1-((8-(2,2′-dichloro-3′-(6-methoxy-5-(((((S)-5-oxopyrrolidin-2-yl)methyl)amino)methyl)pyridin-2-yl)-[1,1′-biphenyl]-3-yl)-2-methyl-4-oxo-4H-pyrido[1,2-a]pyrimidin-3-yl)methyl)pyrrolidine-3-carboxylate;
methyl (R)-1-((8-(2,2′-dichloro-3′-(6-methoxy-5-(((((S)-5-oxopyrrolidin-2-yl)methyl)amino)methyl)pyridin-2-yl)-[1,1′-biphenyl]-3-yl)-2-methyl-4-oxo-4H-pyrido[1,2-a]pyrimidin-3-yl)methyl)pyrrolidine-3-carboxylate;
methyl (S)-1-((8-(2,2′-dichloro-3′-(6-methoxy-5-(((((R)-5-oxopyrrolidin-2-yl)methyl)amino)methyl)pyridin-2-yl)-[1,1′-biphenyl]-3-yl)-2-methyl-4-oxo-4H-pyrido[1,2-a]pyrimidin-3-yl)methyl)pyrrolidine-3-carboxylate;
methyl (R)-1-((8-(2,2′-dichloro-3′-(6-methoxy-5-(((((R)-5-oxopyrrolidin-2-yl)methyl)amino)methyl)pyridin-2-yl)-[1,1′-biphenyl]-3-yl)-2-methyl-4-oxo-4H-pyrido[1,2-a]pyrimidin-3-yl)methyl)pyrrolidine-3-carboxylate;
(S)-8-(2,2′-dichloro-3′-(6-methoxy-5-((((5-oxopyrrolidin-2-yl)methyl)amino)methyl)pyridin-2-yl)-[1,1′-biphenyl]-3-yl)-3-(((2-(1,1-dioxidothiomorpholino)ethyl)amino)methyl)-4H-pyrido[1,2-a]pyrimidin-4-one;
(R)-8-(2,2′-dichloro-3′-(6-methoxy-5-((((5-oxopyrrolidin-2-yl)methyl)amino)methyl)pyridin-2-yl)-[1,1′-biphenyl]-3-yl)-3-(((2-(1,1-dioxidothiomorpholino)ethyl)amino)methyl)-4H-pyrido[1,2-a]pyrimidin-4-one;
(S)-5-((((6-(2,2′-dichloro-3′-(4-(2-hydroxyethyl)-2,3,4,5-tetrahydropyrido[3,2-f1][1,4]oxazepin-8-yl)-[1,1′-biphenyl]-3-yl)-2-methoxypyridin-3-yl)methyl)amino)methyl)pyrrolidin-2-one;
(R)-5-((((6-(2,2′-dichloro-3′-(4-(2-hydroxyethyl)-2,3,4,5-tetrahydropyrido[3,2-f1][1,4]oxazepin-8-yl)-[1,1′-biphenyl]-3-yl)-2-methoxypyridin-3-yl)methyl)amino)methyl)pyrrolidin-2-one;
(S)-8-(2,2′-dichloro-3′-(4-(2-hydroxyethyl)-2,3,4,5-tetrahydropyrido[3,2-f][1,4]oxazepin-8-yl)-[1,1′-biphenyl]-3-yl)-3-((((5-oxopyrrolidin-2-yl)methyl)amino)methyl)-4H-pyrido[1,2-a]pyrimidin-4-one;
(R)-8-(2,2′-dichloro-3′-(4-(2-hydroxyethyl)-2,3,4,5-tetrahydropyrido[3,2-f][1,4]oxazepin-8-yl)-[1,1′-biphenyl]-3-yl)-3-((((5-oxopyrrolidin-2-yl)methyl)amino)methyl)-4H-pyrido[1,2-a]pyrimidin-4-one;
methyl (S)-((8-(2,2′-dichloro-3′-(6-methoxy-5-((((5-oxopyrrolidin-2-yl)methyl)amino)methyl)pyridin-2-yl)-[1,1′-biphenyl]-3-yl)-2-methyl-4-oxo-4H-pyrido[1,2-a]pyrimidin-3-yl)methyl)glycinate;
methyl (R)-((8-(2,2′-dichloro-3′-(6-methoxy-5-((((5-oxopyrrolidin-2-yl)methyl)amino)methyl)pyridin-2-yl)-[1,1′-biphenyl]-3-yl)-2-methyl-4-oxo-4H-pyrido[1,2-a]pyrimidin-3-yl)methyl)glycinate;
(S)-4-(((8-(2,2′-dichloro-3′-(4-fluoro-6-methoxy-5-(((((S)-5-oxopyrrolidin-2-yl)methyl)amino)methyl)pyridin-2-yl)-[1,1′-biphenyl]-3-yl)-4-oxo-4H-pyrido[1,2-a]pyrimidin-3-yl)methyl)amino)-3-hydroxybutanoic acid;
(R)-4-(((8-(2,2′-dichloro-3′-(4-fluoro-6-methoxy-5-(((((S)-5-oxopyrrolidin-2-yl)methyl)amino)methyl)pyridin-2-yl)-[1,1′-biphenyl]-3-yl)-4-oxo-4H-pyrido[1,2-a]pyrimidin-3-yl)methyl)amino)-3-hydroxybutanoic acid;
(S)-4-(((8-(2,2′-dichloro-3′-(4-fluoro-6-methoxy-5-(((((R)-5-oxopyrrolidin-2-yl)methyl)amino)methyl)pyridin-2-yl)-[1,1′-biphenyl]-3-yl)-4-oxo-4H-pyrido[1,2-a]pyrimidin-3-yl)methyl)amino)-3-hydroxybutanoic acid;
(R)-4-(((8-(2,2′-dichloro-3′-(4-fluoro-6-methoxy-5-(((((R)-5-oxopyrrolidin-2-yl)methyl)amino)methyl)pyridin-2-yl)-[1,1′-biphenyl]-3-yl)-4-oxo-4H-pyrido[1,2-a]pyrimidin-3-yl)methyl)amino)-3-hydroxybutanoic acid;
methyl (S)-4-(((8-(2,2′-dichloro-3′-(4-fluoro-6-methoxy-5-(((((S)-5-oxopyrrolidin-2-yl)methyl)amino)methyl)pyridin-2-yl)-[1,1′-biphenyl]-3-yl)-4-oxo-4H-pyrido[1,2-a]pyrimidin-3-yl)methyl)amino)-3-hydroxybutanoate;
methyl (R)-4-(((8-(2,2′-dichloro-3′-(4-fluoro-6-methoxy-5-(((((S)-5-oxopyrrolidin-2-yl)methyl)amino)methyl)pyridin-2-yl)-[1,1′-biphenyl]-3-yl)-4-oxo-4H-pyrido[1,2-a]pyrimidin-3-yl)methyl)amino)-3-hydroxybutanoate;
methyl (S)-4-(((8-(2,2′-dichloro-3′-(4-fluoro-6-methoxy-5-(((((R)-5-oxopyrrolidin-2-yl)methyl)amino)methyl)pyridin-2-yl)-[1,1′-biphenyl]-3-yl)-4-oxo-4H-pyrido[1,2-a]pyrimidin-3-yl)methyl)amino)-3-hydroxybutanoate;
methyl (R)-4-(((8-(2,2′-dichloro-3′-(4-fluoro-6-methoxy-5-(((((R)-5-oxopyrrolidin-2-yl)methyl)amino)methyl)pyridin-2-yl)-[1,1′-biphenyl]-3-yl)-4-oxo-4H-pyrido[1,2-a]pyrimidin-3-yl)methyl)amino)-3-hydroxybutanoate;
8-(2,2′-dichloro-3′-(4-fluoro-6-methoxy-5-(((((S)-5-oxopyrrolidin-2-yl)methyl)amino)methyl)pyridin-2-yl)-[1,1′-biphenyl]-3-yl)-3-((((S)-2-hydroxypropyl)amino)methyl)-4H-pyrido[1,2-a]pyrimidin-4-one;
8-(2,2′-dichloro-3′-(4-fluoro-6-methoxy-5-(((((R)-5-oxopyrrolidin-2-yl)methyl)amino)methyl)pyridin-2-yl)-[1,1′-biphenyl]-3-yl)-3-((((S)-2-hydroxypropyl)amino)methyl)-4H-pyrido[1,2-a]pyrimidin-4-one;
8-(2,2′-dichloro-3′-(4-fluoro-6-methoxy-5-(((((S)-5-oxopyrrolidin-2-yl)methyl)amino)methyl)pyridin-2-yl)-[1,1′-biphenyl]-3-yl)-3-((((R)-2-hydroxypropyl)amino)methyl)-4H-pyrido[1,2-a]pyrimidin-4-one;
8-(2,2′-dichloro-3′-(4-fluoro-6-methoxy-5-(((((R)-5-oxopyrrolidin-2-yl)methyl)amino)methyl)pyridin-2-yl)-[1,1′-biphenyl]-3-yl)-3-((((R)-2-hydroxypropyl)amino)methyl)-4H-pyrido[1,2-a]pyrimidin-4-one;
(S)-8-(2,2′-dichloro-3′-(6-methoxy-5-((((5-oxopyrrolidin-2-yl)methyl)amino)methyl)pyridin-2-yl)-[1,1′-biphenyl]-3-yl)-3-(((3-hydroxy-2,2-dimethylpropyl)amino)methyl)-4H-pyrido[1,2-a]pyrimidin-4-one;
(R)-8-(2,2′-dichloro-3′-(6-methoxy-5-((((5-oxopyrrolidin-2-yl)methyl)amino)methyl)pyridin-2-yl)-[1,1′-biphenyl]-3-yl)-3-(((3-hydroxy-2,2-dimethylpropyl)amino)methyl)-4H-pyrido[1,2-a]pyrimidin-4-one;
3-((((2S)-2-cyclopropyl-2-hydroxyethyl)amino)methyl)-8-(2,2′-dichloro-3′-(6-methoxy-5-(((((S)-5-oxopyrrolidin-2-yl)methyl)amino)methyl)pyridin-2-yl)-[1,1′-biphenyl]-3-yl)-4H-pyrido[1,2-a]pyrimidin-4-one;
3-((((2R)-2-cyclopropyl-2-hydroxyethyl)amino)methyl)-8-(2,2′-dichloro-3′-(6-methoxy-5-(((((S)-5-oxopyrrolidin-2-yl)methyl)amino)methyl)pyridin-2-yl)-[1,1′-biphenyl]-3-yl)-4H-pyrido[1,2-a]pyrimidin-4-one;
3-((((2S)-2-cyclopropyl-2-hydroxyethyl)amino)methyl)-8-(2,2′-dichloro-3′-(6-methoxy-5-(((((R)-5-oxopyrrolidin-2-yl)methyl)amino)methyl)pyridin-2-yl)-[1,1′-biphenyl]-3-yl)-4H-pyrido[1,2-a]pyrimidin-4-one;
3-((((2R)-2-cyclopropyl-2-hydroxyethyl)amino)methyl)-8-(2,2′-dichloro-3′-(6-methoxy-5-(((((R)-5-oxopyrrolidin-2-yl)methyl)amino)methyl)pyridin-2-yl)-[1,1′-biphenyl]-3-yl)-4H-pyrido[1,2-a]pyrimidin-4-one;
(S)-4-(((8-(2,2′-dichloro-3′-(4-fluoro-6-methoxy-5-(((((S)-5-oxopyrrolidin-2-yl)methyl)amino)methyl)pyridin-2-yl)-[1,1′-biphenyl]-3-yl)-4-oxo-4H-pyrido[1,2-a]pyrimidin-3-yl)methyl)amino)-3-hydroxybutanamide;
(R)-4-(((8-(2,2′-dichloro-3′-(4-fluoro-6-methoxy-5-(((((S)-5-oxopyrrolidin-2-yl)methyl)amino)methyl)pyridin-2-yl)-[1,1′-biphenyl]-3-yl)-4-oxo-4H-pyrido[1,2-a]pyrimidin-3-yl)methyl)amino)-3-hydroxybutanamide;
(S)-4-(((8-(2,2′-dichloro-3′-(4-fluoro-6-methoxy-5-(((((R)-5-oxopyrrolidin-2-yl)methyl)amino)methyl)pyridin-2-yl)-[1,1′-biphenyl]-3-yl)-4-oxo-4H-pyrido[1,2-a]pyrimidin-3-yl)methyl)amino)-3-hydroxybutanamide;
(R)-4-(((8-(2,2′-dichloro-3′-(4-fluoro-6-methoxy-5-(((((R)-5-oxopyrrolidin-2-yl)methyl)amino)methyl)pyridin-2-yl)-[1,1′-biphenyl]-3-yl)-4-oxo-4H-pyrido[1,2-a]pyrimidin-3-yl)methyl)amino)-3-hydroxybutanamide;
8-(2,2′-dichloro-3′-(6-methoxy-5-((methyl(3-(methylamino)propyl)amino)methyl)pyridin-2-yl)-[1,1′-biphenyl]-3-yl)-3-((methyl(2-(methylamino)ethyl)amino)methyl)-4H-pyrido[1,2-a]pyrimidin-4-one;
1 tert-butyl (2-(((6-(3′-(3-(((2-((tert-butoxycarbonyl)amino)ethyl)(methyl)amino)methyl)-4-oxo-4H-pyrido[1,2-a]pyrimidin-8-yl)-2,2′-dichloro-[1,1′-biphenyl]-3-yl)-2-methoxypyridin-3-yl)methyl)(methyl)amino)ethyl)carbamate;
(S)-8-(2,2′-dichloro-3′-(6-methoxy-5-((((5-oxopyrrolidin-2-yl)methyl)amino)methyl)pyridin-2-yl)-[1,1′-biphenyl]-3-yl)-3-((methyl(2-(methylamino)ethyl)amino)methyl)-4H-pyrido[1,2-a]pyrimidin-4-one;
(R)-8-(2,2′-dichloro-3′-(6-methoxy-5-((((5-oxopyrrolidin-2-yl)methyl)amino)methyl)pyridin-2-yl)-[1,1′-biphenyl]-3-yl)-3-((methyl(2-(methylamino)ethyl)amino)methyl)-4H-pyrido[1,2-a]pyrimidin-4-one;
(S)-3-(((2-aminoethyl)(methyl)amino)methyl)-8-(2,2′-dichloro-3′-(6-methoxy-5-((((5-oxopyrrolidin-2-yl)methyl)amino)methyl)pyridin-2-yl)-[1,1′-biphenyl]-3-yl)-4H-pyrido[1,2-a]pyrimidin-4-one;
(R)-3-(((2-aminoethyl)(methyl)amino)methyl)-8-(2,2′-dichloro-3′-(6-methoxy-5-((((5-oxopyrrolidin-2-yl)methyl)amino)methyl)pyridin-2-yl)-[1,1′-biphenyl]-3-yl)-4H-pyrido[1,2-a]pyrimidin-4-one;
3-((((2S)-2-cyclopropyl-2-hydroxypropyl)amino)methyl)-8-(2,2′-dichloro-3′-(6-methoxy-5-(((((S)-5-oxopyrrolidin-2-yl)methyl)amino)methyl)pyridin-2-yl)-[1,1′-biphenyl]-3-yl)-4H-pyrido[1,2-a]pyrimidin-4-one;
3-((((2S)-2-cyclopropyl-2-hydroxypropyl)amino)methyl)-8-(2,2′-dichloro-3′-(6-methoxy-5-(((((R)-5-oxopyrrolidin-2-yl)methyl)amino)methyl)pyridin-2-yl)-[1,1′-biphenyl]-3-yl)-4H-pyrido[1,2-a]pyrimidin-4-one;
3-((((2R)-2-cyclopropyl-2-hydroxypropyl)amino)methyl)-8-(2,2′-dichloro-3′-(6-methoxy-5-(((((S)-5-oxopyrrolidin-2-yl)methyl)amino)methyl)pyridin-2-yl)-[1,1′-biphenyl]-3-yl)-4H-pyrido[1,2-a]pyrimidin-4-one;
3-((((2R)-2-cyclopropyl-2-hydroxypropyl)amino)methyl)-8-(2,2′-dichloro-3′-(6-methoxy-5-(((((R)-5-oxopyrrolidin-2-yl)methyl)amino)methyl)pyridin-2-yl)-[1,1′-biphenyl]-3-yl)-4H-pyrido[1,2-a]pyrimidin-4-one;
(S)-8-(2,2′-dichloro-3′-(6-methoxy-5-((((5-oxopyrrolidin-2-yl)methyl)amino)methyl)pyridin-2-yl)-[1,1′-biphenyl]-3-yl)-3-(((2-hydroxyethyl)(methyl)amino)methyl)-4H-pyrido[1,2-a]pyrimidin-4-one;
(R)-8-(2,2′-dichloro-3′-(6-methoxy-5-((((5-oxopyrrolidin-2-yl)methyl)amino)methyl)pyridin-2-yl)-[1,1′-biphenyl]-3-yl)-3-(((2-hydroxyethyl)(methyl)amino)methyl)-4H-pyrido[1,2-a]pyrimidin-4-one;
(S)-5-((((6-(2,2′-dichloro-3′-(3-(((((S)-5-oxopyrrolidin-2-yl)methyl)amino)methyl)pyrazolo[1,5-a]pyrimidin-5-yl)-[1,1′-biphenyl]-3-yl)-2-methoxypyridin-3-yl)methyl)amino)methyl)pyrrolidin-2-one;
(R)-5-((((6-(2,2′-dichloro-3′-(3-(((((S)-5-oxopyrrolidin-2-yl)methyl)amino)methyl)pyrazolo[1,5-a]pyrimidin-5-yl)-[1,1′-biphenyl]-3-yl)-2-methoxypyridin-3-yl)methyl)amino)methyl)pyrrolidin-2-one;
(S)-5-((((6-(2,2′-dichloro-3′-(3-(((((R)-5-oxopyrrolidin-2-yl)methyl)amino)methyl)pyrazolo[1,5-a]pyrimidin-5-yl)-[1,1′-biphenyl]-3-yl)-2-methoxypyridin-3-yl)methyl)amino)methyl)pyrrolidin-2-one;
(R)-5-((((6-(2,2′-dichloro-3′-(3-(((((R)-5-oxopyrrolidin-2-yl)methyl)amino)methyl)pyrazolo[1,5-a]pyrimidin-5-yl)-[1,1′-biphenyl]-3-yl)-2-methoxypyridin-3-yl)methyl)amino)methyl)pyrrolidin-2-one;
(S)-8-(3′-(5-(1-(aminomethyl)cyclopropyl)-6-methoxypyridin-2-yl)-2,2′-dichloro-[1,1′-biphenyl]-3-yl)-3-((((5-oxopyrrolidin-2-yl)methyl)amino)methyl)-4H-pyrido[1,2-a]pyrimidin-4-one;
(R)-8-(3′-(5-(1-(aminomethyl)cyclopropyl)-6-methoxypyridin-2-yl)-2,2′-dichloro-[1,1′-biphenyl]-3-yl)-3-((((5-oxopyrrolidin-2-yl)methyl)amino)methyl)-4H-pyrido[1,2-a]pyrimidin-4-one;
8-(2,2′-dichloro-3′-(6-methoxy-5-(((((S)-5-oxopyrrolidin-2-yl)methyl)amino)methyl)pyridin-2-yl)-[1,1′-biphenyl]-3-yl)-3-(((((S)-5-oxopyrrolidin-3-yl)methyl)amino)methyl)-4H-pyrido[1,2-a]pyrimidin-4-one;
8-(2,2′-dichloro-3′-(6-methoxy-5-(((((R)-5-oxopyrrolidin-2-yl)methyl)amino)methyl)pyridin-2-yl)-[1,1′-biphenyl]-3-yl)-3-(((((S)-5-oxopyrrolidin-3-yl)methyl)amino)methyl)-4H-pyrido[1,2-a]pyrimidin-4-one;
8-(2,2′-dichloro-3′-(6-methoxy-5-(((((S)-5-oxopyrrolidin-2-yl)methyl)amino)methyl)pyridin-2-yl)-[1,1′-biphenyl]-3-yl)-3-(((((R)-5-oxopyrrolidin-3-yl)methyl)amino)methyl)-4H-pyrido[1,2-a]pyrimidin-4-one;
8-(2,2′-dichloro-3′-(6-methoxy-5-(((((R)-5-oxopyrrolidin-2-yl)methyl)amino)methyl)pyridin-2-yl)-[1,1′-biphenyl]-3-yl)-3-(((((R)-5-oxopyrrolidin-3-yl)methyl)amino)methyl)-4H-pyrido[1,2-a]pyrimidin-4-one;
8-(2,2′-dichloro-3′-(6-methoxy-5-(((((S)-5-oxopyrrolidin-2-yl)methyl)amino)methyl)pyridin-2-yl)-[1,1′-biphenyl]-3-yl)-3-((((3R,4S)-3-hydroxytetrahydro-2H-pyran-4-yl)amino)methyl)-2-methyl-4H-pyrido[1,2-a]pyrimidin-4-one;
8-(2,2′-dichloro-3′-(6-methoxy-5-(((((S)-5-oxopyrrolidin-2-yl)methyl)amino)methyl)pyridin-2-yl)-[1,1′-biphenyl]-3-yl)-3-((((3S,4S)-3-hydroxytetrahydro-2H-pyran-4-yl)amino)methyl)-2-methyl-4H-pyrido[1,2-a]pyrimidin-4-one;
8-(2,2′-dichloro-3′-(6-methoxy-5-(((((S)-5-oxopyrrolidin-2-yl)methyl)amino)methyl)pyridin-2-yl)-[1,1′-biphenyl]-3-yl)-3-((((3R,4R)-3-hydroxytetrahydro-2H-pyran-4-yl)amino)methyl)-2-methyl-4H-pyrido[1,2-a]pyrimidin-4-one;
8-(2,2′-dichloro-3′-(6-methoxy-5-(((((S)-5-oxopyrrolidin-2-yl)methyl)amino)methyl)pyridin-2-yl)-[1,1′-biphenyl]-3-yl)-3-((((3S,4R)-3-hydroxytetrahydro-2H-pyran-4-yl)amino)methyl)-2-methyl-4H-pyrido[1,2-a]pyrimidin-4-one;
8-(2,2′-dichloro-3′-(6-methoxy-5-(((((R)-5-oxopyrrolidin-2-yl)methyl)amino)methyl)pyridin-2-yl)-[1,1′-biphenyl]-3-yl)-3-((((3R,4S)-3-hydroxytetrahydro-2H-pyran-4-yl)amino)methyl)-2-methyl-4H-pyrido[1,2-a]pyrimidin-4-one;
8-(2,2′-dichloro-3′-(6-methoxy-5-(((((R)-5-oxopyrrolidin-2-yl)methyl)amino)methyl)pyridin-2-yl)-[1,1′-biphenyl]-3-yl)-3-((((3S,4S)-3-hydroxytetrahydro-2H-pyran-4-yl)amino)methyl)-2-methyl-4H-pyrido[1,2-a]pyrimidin-4-one;
8-(2,2′-dichloro-3′-(6-methoxy-5-(((((R)-5-oxopyrrolidin-2-yl)methyl)amino)methyl)pyridin-2-yl)-[1,1′-biphenyl]-3-yl)-3-((((3R,4R)-3-hydroxytetrahydro-2H-pyran-4-yl)amino)methyl)-2-methyl-4H-pyrido[1,2-a]pyrimidin-4-one;
8-(2,2′-dichloro-3′-(6-methoxy-5-(((((R)-5-oxopyrrolidin-2-yl)methyl)amino)methyl)pyridin-2-yl)-[1,1′-biphenyl]-3-yl)-3-((((3S,4R)-3-hydroxytetrahydro-2H-pyran-4-yl)amino)methyl)-2-methyl-4H-pyrido[1,2-a]pyrimidin-4-one;
8-(2,2′-dichloro-3′-(5-((((3R,4R)-3-hydroxytetrahydro-2H-pyran-4-yl)amino)methyl)-6-methoxypyridin-2-yl)-[1,1′-biphenyl]-3-yl)-2-methyl-3-(((((S)-5-oxopyrrolidin-2-yl)methyl)amino)methyl)-4H-pyrido[1,2-a]pyrimidin-4-one;
8-(2,2′-dichloro-3′-(5-((((3R,4S)-3-hydroxytetrahydro-2H-pyran-4-yl)amino)methyl)-6-methoxypyridin-2-yl)-[1,1′-biphenyl]-3-yl)-2-methyl-3-(((((S)-5-oxopyrrolidin-2-yl)methyl)amino)methyl)-4H-pyrido[1,2-a]pyrimidin-4-one;
8-(2,2′-dichloro-3′-(5-((((3S,4R)-3-hydroxytetrahydro-2H-pyran-4-yl)amino)methyl)-6-methoxypyridin-2-yl)-[1,1′-biphenyl]-3-yl)-2-methyl-3-(((((S)-5-oxopyrrolidin-2-yl)methyl)amino)methyl)-4H-pyrido[1,2-a]pyrimidin-4-one;
8-(2,2′-dichloro-3′-(5-((((3S,4S)-3-hydroxytetrahydro-2H-pyran-4-yl)amino)methyl)-6-methoxypyridin-2-yl)-[1,1′-biphenyl]-3-yl)-2-methyl-3-(((((S)-5-oxopyrrolidin-2-yl)methyl)amino)methyl)-4H-pyrido[1,2-a]pyrimidin-4-one;
8-(2,2′-dichloro-3′-(5-((((3R,4R)-3-hydroxytetrahydro-2H-pyran-4-yl)amino)methyl)-6-methoxypyridin-2-yl)-[1,1′-biphenyl]-3-yl)-2-methyl-3-(((((R)-5-oxopyrrolidin-2-yl)methyl)amino)methyl)-4H-pyrido[1,2-a]pyrimidin-4-one;
8-(2,2′-dichloro-3′-(5-((((3R,4S)-3-hydroxytetrahydro-2H-pyran-4-yl)amino)methyl)-6-methoxypyridin-2-yl)-[1,1′-biphenyl]-3-yl)-2-methyl-3-(((((R)-5-oxopyrrolidin-2-yl)methyl)amino)methyl)-4H-pyrido[1,2-a]pyrimidin-4-one;
8-(2,2′-dichloro-3′-(5-((((3S,4R)-3-hydroxytetrahydro-2H-pyran-4-yl)amino)methyl)-6-methoxypyridin-2-yl)-[1,1′-biphenyl]-3-yl)-2-methyl-3-(((((R)-5-oxopyrrolidin-2-yl)methyl)amino)methyl)-4H-pyrido[1,2-a]pyrimidin-4-one;
8-(2,2′-dichloro-3′-(5-((((3S,4S)-3-hydroxytetrahydro-2H-pyran-4-yl)amino)methyl)-6-methoxypyridin-2-yl)-[1,1′-biphenyl]-3-yl)-2-methyl-3-(((((R)-5-oxopyrrolidin-2-yl)methyl)amino)methyl)-4H-pyrido[1,2-a]pyrimidin-4-one;
(S)-8-(2,2′-dichloro-3′-(6-methoxy-5-((((5-oxopyrrolidin-2-yl)methyl)amino)methyl)pyridin-2-yl)-[1,1′-biphenyl]-3-yl)-3-(((2-hydroxyethyl)amino)methyl)-2-methyl-4H-pyrido[1,2-a]pyrimidin-4-one;
(R)-8-(2,2′-dichloro-3′-(6-methoxy-5-((((5-oxopyrrolidin-2-yl)methyl)amino)methyl)pyridin-2-yl)-[1,1′-biphenyl]-3-yl)-3-(((2-hydroxyethyl)amino)methyl)-2-methyl-4H-pyrido[1,2-a]pyrimidin-4-one;
7-(2,2′-dichloro-3′-(6-methoxy-5-(((((S)-5-oxopyrrolidin-2-yl)methyl)amino)methyl)pyridin-2-yl)-[1,1′-biphenyl]-3-yl)-9-methyl-2-(((((S)-5-oxopyrrolidin-2-yl)methyl)amino)methyl)-4H-pyrido[1,2-a]pyrimidin-4-one;
7-(2,2′-dichloro-3′-(6-methoxy-5-(((((R)-5-oxopyrrolidin-2-yl)methyl)amino)methyl)pyridin-2-yl)-[1,1′-biphenyl]-3-yl)-9-methyl-2-(((((S)-5-oxopyrrolidin-2-yl)methyl)amino)methyl)-4H-pyrido[1,2-a]pyrimidin-4-one;
7-(2,2′-dichloro-3′-(6-methoxy-5-(((((S)-5-oxopyrrolidin-2-yl)methyl)amino)methyl)pyridin-2-yl)-[1,1′-biphenyl]-3-yl)-9-methyl-2-(((((R)-5-oxopyrrolidin-2-yl)methyl)amino)methyl)-4H-pyrido[1,2-a]pyrimidin-4-one;
7-(2,2′-dichloro-3′-(6-methoxy-5-(((((R)-5-oxopyrrolidin-2-yl)methyl)amino)methyl)pyridin-2-yl)-[1,1′-biphenyl]-3-yl)-9-methyl-2-(((((R)-5-oxopyrrolidin-2-yl)methyl)amino)methyl)-4H-pyrido[1,2-a]pyrimidin-4-one;
methyl 1-((6-(2,2′-dichloro-3′-(3-((3-(methoxycarbonyl)azetidin-1-yl)methyl)-1,6-naphthyridin-7-yl)-[1,1′-biphenyl]-3-yl)-2-methoxypyridin-3-yl)methyl)azetidine-3-carboxylate;
8-(2,2′-dichloro-3′-(6-methoxy-5-(((((S)-5-oxopyrrolidin-2-yl)methyl)amino)methyl)pyridin-2-yl)-[1,1′-biphenyl]-3-yl)-3-(5-hydroxy-1,4,5,6-tetrahydropyrimidin-2-yl)-2-methyl-4H-pyrido[1,2-a]pyrimidin-4-one;
8-(2,2′-dichloro-3′-(6-methoxy-5-(((((R)-5-oxopyrrolidin-2-yl)methyl)amino)methyl)pyridin-2-yl)-[1,1′-biphenyl]-3-yl)-3-(5-hydroxy-1,4,5,6-tetrahydropyrimidin-2-yl)-2-methyl-4H-pyrido[1,2-a]pyrimidin-4-one;
methyl (S)-1-((8-(2,2′-dichloro-3′-(6-methoxy-5-((((5-oxopyrrolidin-2-yl)methyl)amino)methyl)pyridin-2-yl)-[1,1′-biphenyl]-3-yl)-6-methyl-4-oxo-4H-pyrido[1,2-a]pyrimidin-3-yl)methyl)azetidine-3-carboxylate;
methyl (R)-1-((8-(2,2′-dichloro-3′-(6-methoxy-5-((((5-oxopyrrolidin-2-yl)methyl)amino)methyl)pyridin-2-yl)-[1,1′-biphenyl]-3-yl)-6-methyl-4-oxo-4H-pyrido[1,2-a]pyrimidin-3-yl)methyl)azetidine-3-carboxylate;
(S)-4-(((8-(2,2′-dichloro-3′-(6-methoxy-5-(((((S)-5-oxopyrrolidin-2-yl)methyl)amino)methyl)pyridin-2-yl)-[1,1′-biphenyl]-3-yl)-4-oxo-4H-pyrido[1,2-a]pyrimidin-3-yl)methyl)amino)-3-hydroxy-N-methylbutanamide;
(R)-4-(((8-(2,2′-dichloro-3′-(6-methoxy-5-(((((S)-5-oxopyrrolidin-2-yl)methyl)amino)methyl)pyridin-2-yl)-[1,1′-biphenyl]-3-yl)-4-oxo-4H-pyrido[1,2-a]pyrimidin-3-yl)methyl)amino)-3-hydroxy-N-methylbutanamide;
(S)-4-(((8-(2,2′-dichloro-3′-(6-methoxy-5-(((((R)-5-oxopyrrolidin-2-yl)methyl)amino)methyl)pyridin-2-yl)-[1,1′-biphenyl]-3-yl)-4-oxo-4H-pyrido[1,2-a]pyrimidin-3-yl)methyl)amino)-3-hydroxy-N-methylbutanamide;
(R)-4-(((8-(2,2′-dichloro-3′-(6-methoxy-5-(((((R)-5-oxopyrrolidin-2-yl)methyl)amino)methyl)pyridin-2-yl)-[1,1′-biphenyl]-3-yl)-4-oxo-4H-pyrido[1,2-a]pyrimidin-3-yl)methyl)amino)-3-hydroxy-N-methylbutanamide;
(S)-4-(((8-(2,2′-dichloro-3′-(4-fluoro-6-methoxy-5-(((((S)-5-oxopyrrolidin-2-yl)methyl)amino)methyl)pyridin-2-yl)-[1,1′-biphenyl]-3-yl)-4-oxo-4H-pyrido[1,2-a]pyrimidin-3-yl)methyl)amino)-3-hydroxy-N-methylbutanamide;
(R)-4-(((8-(2,2′-dichloro-3′-(4-fluoro-6-methoxy-5-(((((S)-5-oxopyrrolidin-2-yl)methyl)amino)methyl)pyridin-2-yl)-[1,1′-biphenyl]-3-yl)-4-oxo-4H-pyrido[1,2-a]pyrimidin-3-yl)methyl)amino)-3-hydroxy-N-methylbutanamide;
(S)-4-(((8-(2,2′-dichloro-3′-(4-fluoro-6-methoxy-5-(((((R)-5-oxopyrrolidin-2-yl)methyl)amino)methyl)pyridin-2-yl)-[1,1′-biphenyl]-3-yl)-4-oxo-4H-pyrido[1,2-a]pyrimidin-3-yl)methyl)amino)-3-hydroxy-N-methylbutanamide;
(R)-4-(((8-(2,2′-dichloro-3′-(4-fluoro-6-methoxy-5-(((((R)-5-oxopyrrolidin-2-yl)methyl)amino)methyl)pyridin-2-yl)-[1,1′-biphenyl]-3-yl)-4-oxo-4H-pyrido[1,2-a]pyrimidin-3-yl)methyl)amino)-3-hydroxy-N-methylbutanamide;
methyl (S)-((8-(2,2′-dichloro-3′-(6-methoxy-5-((((5-oxopyrrolidin-2-yl)methyl)amino)methyl)pyridin-2-yl)-[1,1′-biphenyl]-3-yl)-6-methyl-4-oxo-4H-pyrido[1,2-a]pyrimidin-3-yl)methyl)glycinate;
methyl (R)-((8-(2,2′-dichloro-3′-(6-methoxy-5-((((5-oxopyrrolidin-2-yl)methyl)amino)methyl)pyridin-2-yl)-[1,1′-biphenyl]-3-yl)-6-methyl-4-oxo-4H-pyrido[1,2-a]pyrimidin-3-yl)methyl)glycinate;
methyl (S)-1-((8-(2,2′-dichloro-3′-(6-methoxy-5-(((((S)-5-oxopyrrolidin-2-yl)methyl)amino)methyl)pyridin-2-yl)-[1,1′-biphenyl]-3-yl)-6-methyl-4-oxo-4H-pyrido[1,2-a]pyrimidin-3-yl)methyl)pyrrolidine-3-carboxylate;
methyl (R)-1-((8-(2,2′-dichloro-3′-(6-methoxy-5-(((((S)-5-oxopyrrolidin-2-yl)methyl)amino)methyl)pyridin-2-yl)-[1,1′-biphenyl]-3-yl)-6-methyl-4-oxo-4H-pyrido[1,2-a]pyrimidin-3-yl)methyl)pyrrolidine-3-carboxylate;
methyl (S)-1-((8-(2,2′-dichloro-3′-(6-methoxy-5-(((((R)-5-oxopyrrolidin-2-yl)methyl)amino)methyl)pyridin-2-yl)-[1,1′-biphenyl]-3-yl)-6-methyl-4-oxo-4H-pyrido[1,2-a]pyrimidin-3-yl)methyl)pyrrolidine-3-carboxylate;
methyl (R)-1-((8-(2,2′-dichloro-3′-(6-methoxy-5-(((((R)-5-oxopyrrolidin-2-yl)methyl)amino)methyl)pyridin-2-yl)-[1,1′-biphenyl]-3-yl)-6-methyl-4-oxo-4H-pyrido[1,2-a]pyrimidin-3-yl)methyl)pyrrolidine-3-carboxylate;
methyl (S)-1-((8-(2,2′-dichloro-3′-(6-methoxy-5-((((5-oxopyrrolidin-2-yl)methyl)amino)methyl)pyridin-2-yl)-[1,1′-biphenyl]-3-yl)-2-methyl-4-oxo-4H-pyrido[1,2-a]pyrimidin-3-yl)methyl)azetidine-3-carboxylate;
methyl (R)-1-((8-(2,2′-dichloro-3′-(6-methoxy-5-((((5-oxopyrrolidin-2-yl)methyl)amino)methyl)pyridin-2-yl)-[1,1′-biphenyl]-3-yl)-2-methyl-4-oxo-4H-pyrido[1,2-a]pyrimidin-3-yl)methyl)azetidine-3-carboxylate;
(S)-8-(2,2′-dichloro-3′-(6-methoxy-5-((((5-oxopyrrolidin-2-yl)methyl)amino)methyl)pyridin-2-yl)-[1,1′-biphenyl]-3-yl)-3-(((2,2-difluoroethyl)amino)methyl)-4H-pyrido[1,2-a]pyrimidin-4-one;
(R)-8-(2,2′-dichloro-3′-(6-methoxy-5-((((5-oxopyrrolidin-2-yl)methyl)amino)methyl)pyridin-2-yl)-[1,1′-biphenyl]-3-yl)-3-(((2,2-difluoroethyl)amino)methyl)-4H-pyrido[1,2-a]pyrimidin-4-one;
3-((((S)-2-aminopropyl)amino)methyl)-8-(2,2′-dichloro-3′-(6-methoxy-5-(((((S)-5-oxopyrrolidin-2-yl)methyl)amino)methyl)pyridin-2-yl)-[1,1′-biphenyl]-3-yl)-4H-pyrido[1,2-a]pyrimidin-4-one;
3-((((R)-2-aminopropyl)amino)methyl)-8-(2,2′-dichloro-3′-(6-methoxy-5-(((((S)-5-oxopyrrolidin-2-yl)methyl)amino)methyl)pyridin-2-yl)-[1,1′-biphenyl]-3-yl)-4H-pyrido[1,2-a]pyrimidin-4-one;
3-((((S)-2-aminopropyl)amino)methyl)-8-(2,2′-dichloro-3′-(6-methoxy-5-(((((R)-5-oxopyrrolidin-2-yl)methyl)amino)methyl)pyridin-2-yl)-[1,1′-biphenyl]-3-yl)-4H-pyrido[1,2-a]pyrimidin-4-one;
3-((((R)-2-aminopropyl)amino)methyl)-8-(2,2′-dichloro-3′-(6-methoxy-5-(((((R)-5-oxopyrrolidin-2-yl)methyl)amino)methyl)pyridin-2-yl)-[1,1′-biphenyl]-3-yl)-4H-pyrido[1,2-a]pyrimidin-4-one;
methyl (2-(((8-(2,2′-dichloro-3′-(6-methoxy-5-(((2-((methoxycarbonyl)(methyl)amino)ethyl)(methyl)amino)methyl)pyridin-2-yl)-[1,1′-biphenyl]-3-yl)-4-oxo-4H-pyrido[1,2-a]pyrimidin-3-yl)methyl)(methyl)amino)ethyl)(methyl)carbamate;
(S)-8-(2,2′-dichloro-3′-(6-methoxy-5-((((5-oxopyrrolidin-2-yl)methyl)amino)methyl)pyridin-2-yl)-[1,1′-biphenyl]-3-yl)-3-(((2,2-difluoro-3-hydroxypropyl)amino)methyl)-4H-pyrido[1,2-a]pyrimidin-4-one;
(R)-8-(2,2′-dichloro-3′-(6-methoxy-5-((((5-oxopyrrolidin-2-yl)methyl)amino)methyl)pyridin-2-yl)-[1,1′-biphenyl]-3-yl)-3-(((2,2-difluoro-3-hydroxypropyl)amino)methyl)-4H-pyrido[1,2-a]pyrimidin-4-one;
(S)-8-(2,2′-dichloro-3′-(6-methoxy-5-((((5-oxopyrrolidin-2-yl)methyl)amino)methyl)pyridin-2-yl)-[1,1′-biphenyl]-3-yl)-3-((((1-(hydroxymethyl)cyclopropyl)methyl)amino)methyl)-4H-pyrido[1,2-a]pyrimidin-4-one;
(R)-8-(2,2′-dichloro-3′-(6-methoxy-5-((((5-oxopyrrolidin-2-yl)methyl)amino)methyl)pyridin-2-yl)-[1,1′-biphenyl]-3-yl)-3-((((1-(hydroxymethyl)cyclopropyl)methyl)amino)methyl)-4H-pyrido[1,2-a]pyrimidin-4-one;
8-(2,2′-dichloro-3′-(6-methoxy-5-(((((S)-5-oxopyrrolidin-2-yl)methyl)amino)methyl)pyridin-2-yl)-[1,1′-biphenyl]-3-yl)-3-((((S)-2-methoxypropyl)amino)methyl)-4H-pyrido[1,2-a]pyrimidin-4-one;
8-(2,2′-dichloro-3′-(6-methoxy-5-(((((R)-5-oxopyrrolidin-2-yl)methyl)amino)methyl)pyridin-2-yl)-[1,1′-biphenyl]-3-yl)-3-((((S)-2-methoxypropyl)amino)methyl)-4H-pyrido[1,2-a]pyrimidin-4-one;
8-(2,2′-dichloro-3′-(6-methoxy-5-(((((S)-5-oxopyrrolidin-2-yl)methyl)amino)methyl)pyridin-2-yl)-[1,1′-biphenyl]-3-yl)-3-((((R)-2-methoxypropyl)amino)methyl)-4H-pyrido[1,2-a]pyrimidin-4-one;
8-(2,2′-dichloro-3′-(6-methoxy-5-(((((R)-5-oxopyrrolidin-2-yl)methyl)amino)methyl)pyridin-2-yl)-[1,1′-biphenyl]-3-yl)-3-((((R)-2-methoxypropyl)amino)methyl)-4H-pyrido[1,2-a]pyrimidin-4-one;
(S)-8-(2,2′-dichloro-3′-(6-methoxy-5-((((5-oxopyrrolidin-2-yl)methyl)amino)methyl)pyridin-2-yl)-[1,1′-biphenyl]-3-yl)-3-(((2,2,2-trifluoroethyl)amino)methyl)-4H-pyrido[1,2-a]pyrimidin-4-one;
(R)-8-(2,2′-dichloro-3′-(6-methoxy-5-((((5-oxopyrrolidin-2-yl)methyl)amino)methyl)pyridin-2-yl)-[1,1′-biphenyl]-3-yl)-3-(((2,2,2-trifluoroethyl)amino)methyl)-4H-pyrido[1,2-a]pyrimidin-4-one;
methyl (S)-1-((7-(2,2′-dichloro-3′-(6-methoxy-5-((((5-oxopyrrolidin-2-yl)methyl)amino)methyl)pyridin-2-yl)-[1,1′-biphenyl]-3-yl)-1,6-naphthyridin-3-yl)methyl)azetidine-3-carboxylate;
methyl (R)-1-((7-(2,2′-dichloro-3′-(6-methoxy-5-((((5-oxopyrrolidin-2-yl)methyl)amino)methyl)pyridin-2-yl)-[1,1′-biphenyl]-3-yl)-1,6-naphthyridin-3-yl)methyl)azetidine-3-carboxylate;
methyl (S)-((7-(2,2′-dichloro-3′-(6-methoxy-5-((((5-oxopyrrolidin-2-yl)methyl)amino)methyl)pyridin-2-yl)-[1,1′-biphenyl]-3-yl)-1,6-naphthyridin-3-yl)methyl)glycinate;
methyl (R)-((7-(2,2′-dichloro-3′-(6-methoxy-5-((((5-oxopyrrolidin-2-yl)methyl)amino)methyl)pyridin-2-yl)-[1,1′-biphenyl]-3-yl)-1,6-naphthyridin-3-yl)methyl)glycinate;
8-(2,2′-dichloro-3′-(6-methoxy-5-(((((S)-5-oxopyrrolidin-2-yl)methyl)amino)methyl)pyridin-2-yl)-[1,1′-biphenyl]-3-yl)-3-(((((S)-oxetan-2-yl)methyl)amino)methyl)-4H-pyrido[1,2-a]pyrimidin-4-one;
8-(2,2′-dichloro-3′-(6-methoxy-5-(((((R)-5-oxopyrrolidin-2-yl)methyl)amino)methyl)pyridin-2-yl)-[1,1′-biphenyl]-3-yl)-3-(((((S)-oxetan-2-yl)methyl)amino)methyl)-4H-pyrido[1,2-a]pyrimidin-4-one;
8-(2,2′-dichloro-3′-(6-methoxy-5-(((((S)-5-oxopyrrolidin-2-yl)methyl)amino)methyl)pyridin-2-yl)-[1,1′-biphenyl]-3-yl)-3-(((((R)-oxetan-2-yl)methyl)amino)methyl)-4H-pyrido[1,2-a]pyrimidin-4-one;
8-(2,2′-dichloro-3′-(6-methoxy-5-(((((R)-5-oxopyrrolidin-2-yl)methyl)amino)methyl)pyridin-2-yl)-[1,1′-biphenyl]-3-yl)-3-(((((R)-oxetan-2-yl)methyl)amino)methyl)-4H-pyrido[1,2-a]pyrimidin-4-one;
8-(2,2′-dichloro-3′-(6-methoxy-5-(((((S)-5-oxopyrrolidin-2-yl)methyl)amino)methyl)pyridin-2-yl)-[1,1′-biphenyl]-3-yl)-3-(((((S)-tetrahydrofuran-2-yl)methyl)amino)methyl)-4H-pyrido[1,2-a]pyrimidin-4-one;
8-(2,2′-dichloro-3′-(6-methoxy-5-(((((R)-5-oxopyrrolidin-2-yl)methyl)amino)methyl)pyridin-2-yl)-[1,1′-biphenyl]-3-yl)-3-(((((S)-tetrahydrofuran-2-yl)methyl)amino)methyl)-4H-pyrido[1,2-a]pyrimidin-4-one;
8-(2,2′-dichloro-3′-(6-methoxy-5-(((((S)-5-oxopyrrolidin-2-yl)methyl)amino)methyl)pyridin-2-yl)-[1,1′-biphenyl]-3-yl)-3-(((((R)-tetrahydrofuran-2-yl)methyl)amino)methyl)-4H-pyrido[1,2-a]pyrimidin-4-one;
8-(2,2′-dichloro-3′-(6-methoxy-5-(((((R)-5-oxopyrrolidin-2-yl)methyl)amino)methyl)pyridin-2-yl)-[1,1′-biphenyl]-3-yl)-3-(((((R)-tetrahydrofuran-2-yl)methyl)amino)methyl)-4H-pyrido[1,2-a]pyrimidin-4-one;
(S)-8-(2,2′-dichloro-3′-(6-methoxy-5-((((5-oxopyrrolidin-2-yl)methyl)amino)methyl)pyridin-2-yl)-[1,1′-biphenyl]-3-yl)-3-(((2-hydroxyethyl)amino)methyl)-6-methyl-4H-pyrido[1,2-a]pyrimidin-4-one;
(R)-8-(2,2′-dichloro-3′-(6-methoxy-5-((((5-oxopyrrolidin-2-yl)methyl)amino)methyl)pyridin-2-yl)-[1,1′-biphenyl]-3-yl)-3-(((2-hydroxyethyl)amino)methyl)-6-methyl-4H-pyrido[1,2-a]pyrimidin-4-one;
(S)-5-((((6-(2,2′-dichloro-3′-(3-(((((S)-5-oxopyrrolidin-2-yl)methyl)amino)methyl)imidazo[1,2-a]pyrazin-6-yl)-[1,1′-biphenyl]-3-yl)-2-methoxypyridin-3-yl)methyl)amino)methyl)pyrrolidin-2-one;
(R)-5-((((6-(2,2′-dichloro-3′-(3-(((((S)-5-oxopyrrolidin-2-yl)methyl)amino)methyl)imidazo[1,2-a]pyrazin-6-yl)-[1,1′-biphenyl]-3-yl)-2-methoxypyridin-3-yl)methyl)amino)methyl)pyrrolidin-2-one;
(S)-5-((((6-(2,2′-dichloro-3′-(3-(((((R)-5-oxopyrrolidin-2-yl)methyl)amino)methyl)imidazo[1,2-a]pyrazin-6-yl)-[1,1′-biphenyl]-3-yl)-2-methoxypyridin-3-yl)methyl)amino)methyl)pyrrolidin-2-one;
(R)-5-((((6-(2,2′-dichloro-3′-(3-(((((R)-5-oxopyrrolidin-2-yl)methyl)amino)methyl)imidazo[1,2-a]pyrazin-6-yl)-[1,1′-biphenyl]-3-yl)-2-methoxypyridin-3-yl)methyl)amino)methyl)pyrrolidin-2-one;
(S)-5-((((6-(2,2′-dichloro-3′-(3-(((((S)-5-oxopyrrolidin-2-yl)methyl)amino)methyl)pyrazolo[1,5-a]pyrimidin-6-yl)-[1,1′-biphenyl]-3-yl)-2-methoxypyridin-3-yl)methyl)amino)methyl)pyrrolidin-2-one;
(R)-5-((((6-(2,2′-dichloro-3′-(3-(((((S)-5-oxopyrrolidin-2-yl)methyl)amino)methyl)pyrazolo[1,5-a]pyrimidin-6-yl)-[1,1′-biphenyl]-3-yl)-2-methoxypyridin-3-yl)methyl)amino)methyl)pyrrolidin-2-one;
(S)-5-((((6-(2,2′-dichloro-3′-(3-(((((R)-5-oxopyrrolidin-2-yl)methyl)amino)methyl)pyrazolo[1,5-a]pyrimidin-6-yl)-[1,1′-biphenyl]-3-yl)-2-methoxypyridin-3-yl)methyl)amino)methyl)pyrrolidin-2-one;
(R)-5-((((6-(2,2′-dichloro-3′-(3-(((((R)-5-oxopyrrolidin-2-yl)methyl)amino)methyl)pyrazolo[1,5-a]pyrimidin-6-yl)-[1,1′-biphenyl]-3-yl)-2-methoxypyridin-3-yl)methyl)amino)methyl)pyrrolidin-2-one;
(S)-5-((((6-(2,2′-dichloro-3′-(2-(((((S)-5-oxopyrrolidin-2-yl)methyl)amino)methyl)furo[3,2-b]pyridin-6-yl)-[1,1′-biphenyl]-3-yl)-2-methoxypyridin-3-yl)methyl)amino)methyl)pyrrolidin-2-one;
(R)-5-((((6-(2,2′-dichloro-3′-(2-(((((S)-5-oxopyrrolidin-2-yl)methyl)amino)methyl)furo[3,2-b]pyridin-6-yl)-[1,1′-biphenyl]-3-yl)-2-methoxypyridin-3-yl)methyl)amino)methyl)pyrrolidin-2-one;
(S)-5-((((6-(2,2′-dichloro-3′-(2-(((((R)-5-oxopyrrolidin-2-yl)methyl)amino)methyl)furo[3,2-b]pyridin-6-yl)-[1,1′-biphenyl]-3-yl)-2-methoxypyridin-3-yl)methyl)amino)methyl)pyrrolidin-2-one;
(R)-5-((((6-(2,2′-dichloro-3′-(2-(((((R)-5-oxopyrrolidin-2-yl)methyl)amino)methyl)furo[3,2-b]pyridin-6-yl)-[1,1′-biphenyl]-3-yl)-2-methoxypyridin-3-yl)methyl)amino)methyl)pyrrolidin-2-one;
(S)-5-((((6-(2,2′-dichloro-3′-(1-methyl-2-(((((S)-5-oxopyrrolidin-2-yl)methyl)amino)methyl)-1H-imidazo[4,5-b]pyridin-5-yl)-[1,1′-biphenyl]-3-yl)-2-methoxypyridin-3-yl)methyl)amino)methyl)pyrrolidin-2-one;
(R)-5-((((6-(2,2′-dichloro-3′-(1-methyl-2-(((((S)-5-oxopyrrolidin-2-yl)methyl)amino)methyl)-1H-imidazo[4,5-b]pyridin-5-yl)-[1,1′-biphenyl]-3-yl)-2-methoxypyridin-3-yl)methyl)amino)methyl)pyrrolidin-2-one;
(S)-5-((((6-(2,2′-dichloro-3′-(1-methyl-2-(((((R)-5-oxopyrrolidin-2-yl)methyl)amino)methyl)-1H-imidazo[4,5-b]pyridin-5-yl)-[1,1′-biphenyl]-3-yl)-2-methoxypyridin-3-yl)methyl)amino)methyl)pyrrolidin-2-one;
(R)-5-((((6-(2,2′-dichloro-3′-(1-methyl-2-(((((R)-5-oxopyrrolidin-2-yl)methyl)amino)methyl)-1H-imidazo[4,5-b]pyridin-5-yl)-[1,1′-biphenyl]-3-yl)-2-methoxypyridin-3-yl)methyl)amino)methyl)pyrrolidin-2-one;
(1r,3r)-3-(((6-(2,2′-dichloro-3′-(2-((((1r,3r)-3-hydroxycyclobutyl)amino)methyl)-1-methyl-1H-imidazo[4,5-b]pyridin-5-yl)-[1,1′-biphenyl]-3-yl)-2-methoxypyridin-3-yl)methyl)amino)cyclobutan-1-ol;
(1s,3s)-3-(((6-(2,2′-dichloro-3′-(2-((((1r,3r)-3-hydroxycyclobutyl)amino)methyl)-1-methyl-1H-imidazo[4,5-b]pyridin-5-yl)-[1,1′-biphenyl]-3-yl)-2-methoxypyridin-3-yl)methyl)amino)cyclobutan-1-ol;
(1r,3r)-3-(((6-(2,2′-dichloro-3′-(2-((((1s,3s)-3-hydroxycyclobutyl)amino)methyl)-1-methyl-1H-imidazo[4,5-b]pyridin-5-yl)-[1,1′-biphenyl]-3-yl)-2-methoxypyridin-3-yl)methyl)amino)cyclobutan-1-ol;
(1s,3s)-3-(((6-(2,2′-dichloro-3′-(2-((((1s,3s)-3-hydroxycyclobutyl)amino)methyl)-1-methyl-1H-imidazo[4,5-b]pyridin-5-yl)-[1,1′-biphenyl]-3-yl)-2-methoxypyridin-3-yl)methyl)amino)cyclobutan-1-ol;
(S)-5-((((6-(2,2′-dichloro-3′-(3-methyl-2-(((((S)-5-oxopyrrolidin-2-yl)methyl)amino)methyl)-3H-imidazo[4,5-b]pyridin-5-yl)-[1,1′-biphenyl]-3-yl)-2-methoxypyridin-3-yl)methyl)amino)methyl)pyrrolidin-2-one;
(R)-5-((((6-(2,2′-dichloro-3′-(3-methyl-2-(((((S)-5-oxopyrrolidin-2-yl)methyl)amino)methyl)-3H-imidazo[4,5-b]pyridin-5-yl)-[1,1′-biphenyl]-3-yl)-2-methoxypyridin-3-yl)methyl)amino)methyl)pyrrolidin-2-one;
(S)-5-((((6-(2,2′-dichloro-3′-(3-methyl-2-(((((R)-5-oxopyrrolidin-2-yl)methyl)amino)methyl)-3H-imidazo[4,5-b]pyridin-5-yl)-[1,1′-biphenyl]-3-yl)-2-methoxypyridin-3-yl)methyl)amino)methyl)pyrrolidin-2-one;
(R)-5-((((6-(2,2′-dichloro-3′-(3-methyl-2-(((((R)-5-oxopyrrolidin-2-yl)methyl)amino)methyl)-3H-imidazo[4,5-b]pyridin-5-yl)-[1,1′-biphenyl]-3-yl)-2-methoxypyridin-3-yl)methyl)amino)methyl)pyrrolidin-2-one;
(1r,3r)-3-(((6-(2,2′-dichloro-3′-(2-((((1r,3r)-3-hydroxycyclobutyl)amino)methyl)-3-methyl-3H-imidazo[4,5-b]pyridin-5-yl)-[1,1′-biphenyl]-3-yl)-2-methoxypyridin-3-yl)methyl)amino)cyclobutan-1-ol;
(1s,3s)-3-(((6-(2,2′-dichloro-3′-(2-((((1r,3r)-3-hydroxycyclobutyl)amino)methyl)-3-methyl-3H-imidazo[4,5-b]pyridin-5-yl)-[1,1′-biphenyl]-3-yl)-2-methoxypyridin-3-yl)methyl)amino)cyclobutan-1-ol;
(1r,3r)-3-(((6-(2,2′-dichloro-3′-(2-((((1s,3s)-3-hydroxycyclobutyl)amino)methyl)-3-methyl-3H-imidazo[4,5-b]pyridin-5-yl)-[1,1′-biphenyl]-3-yl)-2-methoxypyridin-3-yl)methyl)amino)cyclobutan-1-ol;
(1s,3s)-3-(((6-(2,2′-dichloro-3′-(2-((((1s,3s)-3-hydroxycyclobutyl)amino)methyl)-3-methyl-3H-imidazo[4,5-b]pyridin-5-yl)-[1,1′-biphenyl]-3-yl)-2-methoxypyridin-3-yl)methyl)amino)cyclobutan-1-ol;
(S)-8-(2,2′-dichloro-3′-(6-methoxy-5-((((5-oxopyrrolidin-2-yl)methyl)amino)methyl)pyridin-2-yl)-[1,1′-biphenyl]-3-yl)-3-(((3-hydroxypropyl)amino)methyl)-4H-pyrido[1,2-a]pyrimidin-4-one;
(R)-8-(2,2′-dichloro-3′-(6-methoxy-5-((((5-oxopyrrolidin-2-yl)methyl)amino)methyl)pyridin-2-yl)-[1,1′-biphenyl]-3-yl)-3-(((3-hydroxypropyl)amino)methyl)-4H-pyrido[1,2-a]pyrimidin-4-one;
(S)-8-(2,2′-dichloro-3′-(6-methoxy-5-((((5-oxopyrrolidin-2-yl)methyl)amino)methyl)pyridin-2-yl)-[1,1′-biphenyl]-3-yl)-3-(((3-hydroxypropyl)(methyl)amino)methyl)-4H-pyrido[1,2-a]pyrimidin-4-one;
(R)-8-(2,2′-dichloro-3′-(6-methoxy-5-((((5-oxopyrrolidin-2-yl)methyl)amino)methyl)pyridin-2-yl)-[1,1′-biphenyl]-3-yl)-3-(((3-hydroxypropyl)(methyl)amino)methyl)-4H-pyrido[1,2-a]pyrimidin-4-one;
8-(2,2′-dichloro-3′-(6-methoxy-5-(((((S)-5-oxopyrrolidin-2-yl)methyl)amino)methyl)pyridin-2-yl)-[1,1′-biphenyl]-3-yl)-3-((((1r,3r)-3-hydroxy-3-methylcyclobutyl)(methyl)amino)methyl)-4H-pyrido[1,2-a]pyrimidin-4-one;
8-(2,2′-dichloro-3′-(6-methoxy-5-(((((S)-5-oxopyrrolidin-2-yl)methyl)amino)methyl)pyridin-2-yl)-[1,1′-biphenyl]-3-yl)-3-((((1s,3s)-3-hydroxy-3-methylcyclobutyl)(methyl)amino)methyl)-4H-pyrido[1,2-a]pyrimidin-4-one;
8-(2,2′-dichloro-3′-(6-methoxy-5-(((((R)-5-oxopyrrolidin-2-yl)methyl)amino)methyl)pyridin-2-yl)-[1,1′-biphenyl]-3-yl)-3-((((1r,3r)-3-hydroxy-3-methylcyclobutyl)(methyl)amino)methyl)-4H-pyrido[1,2-a]pyrimidin-4-one;
8-(2,2′-dichloro-3′-(6-methoxy-5-(((((R)-5-oxopyrrolidin-2-yl)methyl)amino)methyl)pyridin-2-yl)-[1,1′-biphenyl]-3-yl)-3-((((1s,3s)-3-hydroxy-3-methylcyclobutyl)(methyl)amino)methyl)-4H-pyrido[1,2-a]pyrimidin-4-one;
8-(2,2′-dichloro-3′-(6-methoxy-5-(((((S)-5-oxopyrrolidin-2-yl)methyl)amino)methyl)pyridin-2-yl)-[1,1′-biphenyl]-3-yl)-3-((((S)-2-hydroxypropyl)(methyl)amino)methyl)-4H-pyrido[1,2-a]pyrimidin-4-one;
8-(2,2′-dichloro-3′-(6-methoxy-5-(((((R)-5-oxopyrrolidin-2-yl)methyl)amino)methyl)pyridin-2-yl)-[1,1′-biphenyl]-3-yl)-3-((((S)-2-hydroxypropyl)(methyl)amino)methyl)-4H-pyrido[1,2-a]pyrimidin-4-one;
8-(2,2′-dichloro-3′-(6-methoxy-5-(((((S)-5-oxopyrrolidin-2-yl)methyl)amino)methyl)pyridin-2-yl)-[1,1′-biphenyl]-3-yl)-3-((((R)-2-hydroxypropyl)(methyl)amino)methyl)-4H-pyrido[1,2-a]pyrimidin-4-one;
8-(2,2′-dichloro-3′-(6-methoxy-5-(((((R)-5-oxopyrrolidin-2-yl)methyl)amino)methyl)pyridin-2-yl)-[1,1′-biphenyl]-3-yl)-3-((((R)-2-hydroxypropyl)(methyl)amino)methyl)-4H-pyrido[1,2-a]pyrimidin-4-one;
ethyl (S)-4-(((8-(2,2′-dichloro-3′-(6-methoxy-5-(((((S)-5-oxopyrrolidin-2-yl)methyl)amino)methyl)pyridin-2-yl)-[1,1′-biphenyl]-3-yl)-4-oxo-4H-pyrido[1,2-a]pyrimidin-3-yl)methyl)amino)-3-hydroxybutanoate;
ethyl (R)-4-(((8-(2,2′-dichloro-3′-(6-methoxy-5-(((((S)-5-oxopyrrolidin-2-yl)methyl)amino)methyl)pyridin-2-yl)-[1,1′-biphenyl]-3-yl)-4-oxo-4H-pyrido[1,2-a]pyrimidin-3-yl)methyl)amino)-3-hydroxybutanoate;
ethyl (S)-4-(((8-(2,2′-dichloro-3′-(6-methoxy-5-(((((R)-5-oxopyrrolidin-2-yl)methyl)amino)methyl)pyridin-2-yl)-[1,1′-biphenyl]-3-yl)-4-oxo-4H-pyrido[1,2-a]pyrimidin-3-yl)methyl)amino)-3-hydroxybutanoate;
ethyl (R)-4-(((8-(2,2′-dichloro-3′-(6-methoxy-5-(((((R)-5-oxopyrrolidin-2-yl)methyl)amino)methyl)pyridin-2-yl)-[1,1′-biphenyl]-3-yl)-4-oxo-4H-pyrido[1,2-a]pyrimidin-3-yl)methyl)amino)-3-hydroxybutanoate;
8-(2,2′-dichloro-3′-(6-methoxy-5-(((2-(2-oxopyrrolidin-1-yl)ethyl)amino)methyl)pyridin-2-yl)-[1,1′-biphenyl]-3-yl)-3-(((2-(2-oxopyrrolidin-1-yl)ethyl)amino)methyl)-4H-pyrido[1,2-a]pyrimidin-4-one;
3-(((1-acetylpiperidin-4-yl)amino)methyl)-8-(3′-(5-(((1-acetylpiperidin-4-yl)amino)methyl)-6-methoxypyridin-2-yl)-2,2′-dichloro-[1,1′-biphenyl]-3-yl)-4H-pyrido[1,2-a]pyrimidin-4-one;
8-(2,2′-dichloro-3′-(6-methoxy-5-(((((S)-5-oxopyrrolidin-2-yl)methyl)amino)methyl)pyridin-2-yl)-[1,1′-biphenyl]-3-yl)-3-(5-hydroxy-1,4,5,6-tetrahydropyrimidin-2-yl)-6-methyl-4H-pyrido[1,2-a]pyrimidin-4-one;
8-(2,2′-dichloro-3′-(6-methoxy-5-(((((R)-5-oxopyrrolidin-2-yl)methyl)amino)methyl)pyridin-2-yl)-[1,1′-biphenyl]-3-yl)-3-(5-hydroxy-1,4,5,6-tetrahydropyrimidin-2-yl)-6-methyl-4H-pyrido[1,2-a]pyrimidin-4-one;
7-(2,2′-dichloro-3′-(6-methoxy-5-(((((S)-5-oxopyrrolidin-2-yl)methyl)amino)methyl)pyridin-2-yl)-[1,1′-biphenyl]-3-yl)-2-((((S)-2-hydroxypropyl)amino)methyl)-4H-pyrido[1,2-a]pyrimidin-4-one;
7-(2,2′-dichloro-3′-(6-methoxy-5-(((((R)-5-oxopyrrolidin-2-yl)methyl)amino)methyl)pyridin-2-yl)-[1,1′-biphenyl]-3-yl)-2-((((S)-2-hydroxypropyl)amino)methyl)-4H-pyrido[1,2-a]pyrimidin-4-one;
7-(2,2′-dichloro-3′-(6-methoxy-5-(((((S)-5-oxopyrrolidin-2-yl)methyl)amino)methyl)pyridin-2-yl)-[1,1′-biphenyl]-3-yl)-2-((((R)-2-hydroxypropyl)amino)methyl)-4H-pyrido[1,2-a]pyrimidin-4-one;
7-(2,2′-dichloro-3′-(6-methoxy-5-(((((R)-5-oxopyrrolidin-2-yl)methyl)amino)methyl)pyridin-2-yl)-[1,1′-biphenyl]-3-yl)-2-((((R)-2-hydroxypropyl)amino)methyl)-4H-pyrido[1,2-a]pyrimidin-4-one;
7-(2,2′-dichloro-3′-(6-methoxy-5-(((((S)-5-oxopyrrolidin-2-yl)methyl)amino)methyl)pyridin-2-yl)-[1,1′-biphenyl]-3-yl)-2-((((1r,3r)-3-hydroxy-3-methylcyclobutyl)amino)methyl)-4H-pyrido[1,2-a]pyrimidin-4-one;
7-(2,2′-dichloro-3′-(6-methoxy-5-(((((S)-5-oxopyrrolidin-2-yl)methyl)amino)methyl)pyridin-2-yl)-[1,1′-biphenyl]-3-yl)-2-((((1s,3s)-3-hydroxy-3-methylcyclobutyl)amino)methyl)-4H-pyrido[1,2-a]pyrimidin-4-one;
7-(2,2′-dichloro-3′-(6-methoxy-5-(((((R)-5-oxopyrrolidin-2-yl)methyl)amino)methyl)pyridin-2-yl)-[1,1′-biphenyl]-3-yl)-2-((((1r,3r)-3-hydroxy-3-methylcyclobutyl)amino)methyl)-4H-pyrido[1,2-a]pyrimidin-4-one;
7-(2,2′-dichloro-3′-(6-methoxy-5-(((((R)-5-oxopyrrolidin-2-yl)methyl)amino)methyl)pyridin-2-yl)-[1,1′-biphenyl]-3-yl)-2-((((1s,3s)-3-hydroxy-3-methylcyclobutyl)amino)methyl)-4H-pyrido[1,2-a]pyrimidin-4-one;
(S)-5-((((6-(2,2′-dichloro-3′-((S)-4-((((S)-5-oxopyrrolidin-2-yl)methyl)amino)chroman-7-yl)-[1,1′-biphenyl]-3-yl)-2-methoxypyridin-3-yl)methyl)amino)methyl)pyrrolidin-2-one;
(S)-5-((((6-(2,2′-dichloro-3′-((R)-4-((((S)-5-oxopyrrolidin-2-yl)methyl)amino)chroman-7-yl)-[1,1′-biphenyl]-3-yl)-2-methoxypyridin-3-yl)methyl)amino)methyl)pyrrolidin-2-one;
(S)-5-((((6-(2,2′-dichloro-3′-((S)-4-((((R)-5-oxopyrrolidin-2-yl)methyl)amino)chroman-7-yl)-[1,1′-biphenyl]-3-yl)-2-methoxypyridin-3-yl)methyl)amino)methyl)pyrrolidin-2-one;
(S)-5-((((6-(2,2′-dichloro-3′-((R)-4-((((R)-5-oxopyrrolidin-2-yl)methyl)amino)chroman-7-yl)-[1,1′-biphenyl]-3-yl)-2-methoxypyridin-3-yl)methyl)amino)methyl)pyrrolidin-2-one;
(R)-5-((((6-(2,2′-dichloro-3′-((S)-4-((((S)-5-oxopyrrolidin-2-yl)methyl)amino)chroman-7-yl)-[1,1′-biphenyl]-3-yl)-2-methoxypyridin-3-yl)methyl)amino)methyl)pyrrolidin-2-one;
(R)-5-((((6-(2,2′-dichloro-3′-((R)-4-((((S)-5-oxopyrrolidin-2-yl)methyl)amino)chroman-7-yl)-[1,1′-biphenyl]-3-yl)-2-methoxypyridin-3-yl)methyl)amino)methyl)pyrrolidin-2-one;
(R)-5-((((6-(2,2′-dichloro-3′-((S)-4-((((R)-5-oxopyrrolidin-2-yl)methyl)amino)chroman-7-yl)-[1,1′-biphenyl]-3-yl)-2-methoxypyridin-3-yl)methyl)amino)methyl)pyrrolidin-2-one;
(R)-5-((((6-(2,2′-dichloro-3′-((R)-4-((((R)-5-oxopyrrolidin-2-yl)methyl)amino)chroman-7-yl)-[1,1′-biphenyl]-3-yl)-2-methoxypyridin-3-yl)methyl)amino)methyl)pyrrolidin-2-one;
(S)-8-(2,2′-dichloro-3′-(6-methoxy-5-((((5-oxopyrrolidin-2-yl)methyl)amino)methyl)pyridin-2-yl)-[1,1′-biphenyl]-3-yl)-3-((5-oxo-1,4-diazepan-1-yl)methyl)-4H-pyrido[1,2-a]pyrimidin-4-one;
(R)-8-(2,2′-dichloro-3′-(6-methoxy-5-((((5-oxopyrrolidin-2-yl)methyl)amino)methyl)pyridin-2-yl)-[1,1′-biphenyl]-3-yl)-3-((5-oxo-1,4-diazepan-1-yl)methyl)-4H-pyrido[1,2-a]pyrimidin-4-one;
(S)-3-((3,3-bis(hydroxymethyl)pyrrolidin-1-yl)methyl)-8-(2,2′-dichloro-3′-(6-methoxy-5-((((5-oxopyrrolidin-2-yl)methyl)amino)methyl)pyridin-2-yl)-[1,1′-biphenyl]-3-yl)-4H-pyrido[1,2-a]pyrimidin-4-one;
(R)-3-((3,3-bis(hydroxymethyl)pyrrolidin-1-yl)methyl)-8-(2,2′-dichloro-3′-(6-methoxy-5-((((5-oxopyrrolidin-2-yl)methyl)amino)methyl)pyridin-2-yl)-[1,1′-biphenyl]-3-yl)-4H-pyrido[1,2-a]pyrimidin-4-one;
(S)-3-((6-acetyl-2,6-diazaspiro[3.3]heptan-2-yl)methyl)-8-(2,2′-dichloro-3′-(6-methoxy-5-((((5-oxopyrrolidin-2-yl)methyl)amino)methyl)pyridin-2-yl)-[1,1′-biphenyl]-3-yl)-4H-pyrido[1,2-a]pyrimidin-4-one;
(R)-3-((6-acetyl-2,6-diazaspiro[3.3]heptan-2-yl)methyl)-8-(2,2′-dichloro-3′-(6-methoxy-5-((((5-oxopyrrolidin-2-yl)methyl)amino)methyl)pyridin-2-yl)-[1,1′-biphenyl]-3-yl)-4H-pyrido[1,2-a]pyrimidin-4-one;
N-((6-(2,2′-dichloro-3′-(6-methoxy-5-(((((S)-5-oxopyrrolidin-2-yl)methyl)amino)methyl)pyridin-2-yl)-[1,1′-biphenyl]-3-yl)-2-methoxypyridin-3-yl)methyl)-N—(((S)-5-oxopyrrolidin-2-yl)methyl)formamide;
N-((6-(2,2′-dichloro-3′-(6-methoxy-5-(((((R)-5-oxopyrrolidin-2-yl)methyl)amino)methyl)pyridin-2-yl)-[1,1′-biphenyl]-3-yl)-2-methoxypyridin-3-yl)methyl)-N—(((S)-5-oxopyrrolidin-2-yl)methyl)formamide;
N-((6-(2,2′-dichloro-3′-(6-methoxy-5-(((((S)-5-oxopyrrolidin-2-yl)methyl)amino)methyl)pyridin-2-yl)-[1,1′-biphenyl]-3-yl)-2-methoxypyridin-3-yl)methyl)-N—(((R)-5-oxopyrrolidin-2-yl)methyl)formamide;
N-((6-(2,2′-dichloro-3′-(6-methoxy-5-(((((R)-5-oxopyrrolidin-2-yl)methyl)amino)methyl)pyridin-2-yl)-[1,1′-biphenyl]-3-yl)-2-methoxypyridin-3-yl)methyl)-N—(((R)-5-oxopyrrolidin-2-yl)methyl)formamide;
6-(2,2′-dichloro-3′-(6-methoxy-5-(((((S)-5-oxopyrrolidin-2-yl)methyl)amino)methyl)pyridin-2-yl)-[1,1′-biphenyl]-3-yl)-3-methyl-2-(((((S)-5-oxopyrrolidin-2-yl)methyl)amino)methyl)pyrrolo[2,1-f][1,2,4]triazin-4(3H)-one;
6-(2,2′-dichloro-3′-(6-methoxy-5-(((((R)-5-oxopyrrolidin-2-yl)methyl)amino)methyl)pyridin-2-yl)-[1,1′-biphenyl]-3-yl)-3-methyl-2-(((((S)-5-oxopyrrolidin-2-yl)methyl)amino)methyl)pyrrolo[2,1-f][1,2,4]triazin-4(3H)-one;
6-(2,2′-dichloro-3′-(6-methoxy-5-(((((S)-5-oxopyrrolidin-2-yl)methyl)amino)methyl)pyridin-2-yl)-[1,1′-biphenyl]-3-yl)-3-methyl-2-(((((R)-5-oxopyrrolidin-2-yl)methyl)amino)methyl)pyrrolo[2,1-f][1,2,4]triazin-4(3H)-one;
6-(2,2′-dichloro-3′-(6-methoxy-5-(((((R)-5-oxopyrrolidin-2-yl)methyl)amino)methyl)pyridin-2-yl)-[1,1′-biphenyl]-3-yl)-3-methyl-2-(((((R)-5-oxopyrrolidin-2-yl)methyl)amino)methyl)pyrrolo[2,1-f][1,2,4]triazin-4(3H)-one;
6-(2,2′-dichloro-3′-(6-methoxy-5-(((((S)-5-oxopyrrolidin-2-yl)methyl)amino)methyl)pyridin-2-yl)-[1,1′-biphenyl]-3-yl)-2-((((1r,3r)-3-hydroxy-3-methylcyclobutyl)amino)methyl)-3-methylpyrrolo[2,1-f][1,2,4]triazin-4(3H)-one;
6-(2,2′-dichloro-3′-(6-methoxy-5-(((((S)-5-oxopyrrolidin-2-yl)methyl)amino)methyl)pyridin-2-yl)-[1,1′-biphenyl]-3-yl)-2-((((1s,3s)-3-hydroxy-3-methylcyclobutyl)amino)methyl)-3-methylpyrrolo[2,1-f][1,2,4]triazin-4(3H)-one;
6-(2,2′-dichloro-3′-(6-methoxy-5-(((((R)-5-oxopyrrolidin-2-yl)methyl)amino)methyl)pyridin-2-yl)-[1,1′-biphenyl]-3-yl)-2-((((1r,3r)-3-hydroxy-3-methylcyclobutyl)amino)methyl)-3-methylpyrrolo[2,1-f][1,2,4]triazin-4(3H)-one;
6-(2,2′-dichloro-3′-(6-methoxy-5-(((((R)-5-oxopyrrolidin-2-yl)methyl)amino)methyl)pyridin-2-yl)-[1,1′-biphenyl]-3-yl)-2-((((1s,3s)-3-hydroxy-3-methylcyclobutyl)amino)methyl)-3-methylpyrrolo[2,1-f][1,2,4]triazin-4(3H)-one;
(S)-5-((((6-(2,2′-dichloro-3′-(3-((((S)-2-hydroxypropyl)amino)methyl)-1,6-naphthyridin-7-yl)-[1,1′-biphenyl]-3-yl)-2-methoxypyridin-3-yl)methyl)amino)methyl)pyrrolidin-2-one;
(S)-5-((((6-(2,2′-dichloro-3′-(3-((((R)-2-hydroxypropyl)amino)methyl)-1,6-naphthyridin-7-yl)-[1,1′-biphenyl]-3-yl)-2-methoxypyridin-3-yl)methyl)amino)methyl)pyrrolidin-2-one;
(R)-5-((((6-(2,2′-dichloro-3′-(3-((((S)-2-hydroxypropyl)amino)methyl)-1,6-naphthyridin-7-yl)-[1,1′-biphenyl]-3-yl)-2-methoxypyridin-3-yl)methyl)amino)methyl)pyrrolidin-2-one;
(R)-5-((((6-(2,2′-dichloro-3′-(3-((((R)-2-hydroxypropyl)amino)methyl)-1,6-naphthyridin-7-yl)-[1,1′-biphenyl]-3-yl)-2-methoxypyridin-3-yl)methyl)amino)methyl)pyrrolidin-2-one;
(S)-5-((((6-(2,2′-dichloro-3′-(3-(((2-hydroxyethyl)amino)methyl)-1,6-naphthyridin-7-yl)-[1,1′-biphenyl]-3-yl)-2-methoxypyridin-3-yl)methyl)amino)methyl)pyrrolidin-2-one;
(R)-5-((((6-(2,2′-dichloro-3′-(3-(((2-hydroxyethyl)amino)methyl)-1,6-naphthyridin-7-yl)-[1,1′-biphenyl]-3-yl)-2-methoxypyridin-3-yl)methyl)amino)methyl)pyrrolidin-2-one;
8-(2,2′-dichloro-3′-(4-chloro-6-methoxy-5-(((((S)-5-oxopyrrolidin-2-yl)methyl)amino)methyl)pyridin-2-yl)-[1,1′-biphenyl]-3-yl)-3-(((((S)-5-oxopyrrolidin-2-yl)methyl)amino)methyl)-4H-pyrido[1,2-a]pyrimidin-4-one;
8-(2,2′-dichloro-3′-(4-chloro-6-methoxy-5-(((((R)-5-oxopyrrolidin-2-yl)methyl)amino)methyl)pyridin-2-yl)-[1,1′-biphenyl]-3-yl)-3-(((((S)-5-oxopyrrolidin-2-yl)methyl)amino)methyl)-4H-pyrido[1,2-a]pyrimidin-4-one;
8-(2,2′-dichloro-3′-(4-chloro-6-methoxy-5-(((((S)-5-oxopyrrolidin-2-yl)methyl)amino)methyl)pyridin-2-yl)-[1,1′-biphenyl]-3-yl)-3-(((((R)-5-oxopyrrolidin-2-yl)methyl)amino)methyl)-4H-pyrido[1,2-a]pyrimidin-4-one;
8-(2,2′-dichloro-3′-(4-chloro-6-methoxy-5-(((((R)-5-oxopyrrolidin-2-yl)methyl)amino)methyl)pyridin-2-yl)-[1,1′-biphenyl]-3-yl)-3-(((((R)-5-oxopyrrolidin-2-yl)methyl)amino)methyl)-4H-pyrido[1,2-a]pyrimidin-4-one;
(S)-4-(((8-(2,2′-dichloro-3′-(4-fluoro-6-methoxy-5-(((((S)-5-oxopyrrolidin-2-yl)methyl)amino)methyl)pyridin-2-yl)-[1,1′-biphenyl]-3-yl)-4-oxo-4H-pyrido[1,2-a]pyrimidin-3-yl)methyl)amino)-3-hydroxy-N,N-dimethylbutanamide;
(R)-4-(((8-(2,2′-dichloro-3′-(4-fluoro-6-methoxy-5-(((((S)-5-oxopyrrolidin-2-yl)methyl)amino)methyl)pyridin-2-yl)-[1,1′-biphenyl]-3-yl)-4-oxo-4H-pyrido[1,2-a]pyrimidin-3-yl)methyl)amino)-3-hydroxy-N,N-dimethylbutanamide;
(S)-4-(((8-(2,2′-dichloro-3′-(4-fluoro-6-methoxy-5-(((((R)-5-oxopyrrolidin-2-yl)methyl)amino)methyl)pyridin-2-yl)-[1,1′-biphenyl]-3-yl)-4-oxo-4H-pyrido[1,2-a]pyrimidin-3-yl)methyl)amino)-3-hydroxy-N,N-dimethylbutanamide;
(R)-4-(((8-(2,2′-dichloro-3′-(4-fluoro-6-methoxy-5-(((((R)-5-oxopyrrolidin-2-yl)methyl)amino)methyl)pyridin-2-yl)-[1,1′-biphenyl]-3-yl)-4-oxo-4H-pyrido[1,2-a]pyrimidin-3-yl)methyl)amino)-3-hydroxy-N,N-dimethylbutanamide;
8-(2,2′-dichloro-3′-(4-chloro-6-methoxy-3-(((((R)-5-oxopyrrolidin-2-yl)methyl)amino)methyl)pyridin-2-yl)-[1,1′-biphenyl]-3-yl)-3-(((((S)-5-oxopyrrolidin-2-yl)methyl)amino)methyl)-4H-pyrido[1,2-a]pyrimidin-4-one;
8-(2,2′-dichloro-3′-(4-chloro-6-methoxy-3-(((((R)-5-oxopyrrolidin-2-yl)methyl)amino)methyl)pyridin-2-yl)-[1,1′-biphenyl]-3-yl)-3-(((((R)-5-oxopyrrolidin-2-yl)methyl)amino)methyl)-4H-pyrido[1,2-a]pyrimidin-4-one;
8-(2,2′-dichloro-3′-(4-chloro-6-methoxy-3-(((((S)-5-oxopyrrolidin-2-yl)methyl)amino)methyl)pyridin-2-yl)-[1,1′-biphenyl]-3-yl)-3-(((((S)-5-oxopyrrolidin-2-yl)methyl)amino)methyl)-4H-pyrido[1,2-a]pyrimidin-4-one;
8-(2,2′-dichloro-3′-(4-chloro-6-methoxy-3-(((((S)-5-oxopyrrolidin-2-yl)methyl)amino)methyl)pyridin-2-yl)-[1,1′-biphenyl]-3-yl)-3-(((((R)-5-oxopyrrolidin-2-yl)methyl)amino)methyl)-4H-pyrido[1,2-a]pyrimidin-4-one;
(S)-5-((((6-(2,2′-dichloro-3′-(2-(((((S)-5-oxopyrrolidin-2-yl)methyl)amino)methyl)imidazo[1,2-b]pyridazin-6-yl)-[1,1′-biphenyl]-3-yl)-2-methoxypyridin-3-yl)methyl)amino)methyl)pyrrolidin-2-one;
(R)-5-((((6-(2,2′-dichloro-3′-(2-(((((S)-5-oxopyrrolidin-2-yl)methyl)amino)methyl)imidazo[1,2-b]pyridazin-6-yl)-[1,1′-biphenyl]-3-yl)-2-methoxypyridin-3-yl)methyl)amino)methyl)pyrrolidin-2-one;
(S)-5-((((6-(2,2′-dichloro-3′-(2-(((((R)-5-oxopyrrolidin-2-yl)methyl)amino)methyl)imidazo[1,2-b]pyridazin-6-yl)-[1,1′-biphenyl]-3-yl)-2-methoxypyridin-3-yl)methyl)amino)methyl)pyrrolidin-2-one;
(R)-5-((((6-(2,2′-dichloro-3′-(2-(((((R)-5-oxopyrrolidin-2-yl)methyl)amino)methyl)imidazo[1,2-b]pyridazin-6-yl)-[1,1′-biphenyl]-3-yl)-2-methoxypyridin-3-yl)methyl)amino)methyl)pyrrolidin-2-one;
2-(((6-(2,2′-dichloro-3′-(2-(((2-hydroxyethyl)(methyl)amino)methyl)imidazo[1,2-b]pyridazin-6-yl)-[1,1′-biphenyl]-3-yl)-2-methoxypyridin-3-yl)methyl)(methyl)amino)ethan-1-ol;
(S)-1-(((6-(2,2′-dichloro-3′-(2-((((S)-2-hydroxypropyl)amino)methyl)imidazo[1,2-b]pyridazin-6-yl)-[1,1′-biphenyl]-3-yl)-2-methoxypyridin-3-yl)methyl)amino)propan-2-ol;
(R)-1-(((6-(2,2′-dichloro-3′-(2-((((S)-2-hydroxypropyl)amino)methyl)imidazo[1,2-b]pyridazin-6-yl)-[1,1′-biphenyl]-3-yl)-2-methoxypyridin-3-yl)methyl)amino)propan-2-ol;
(S)-1-(((6-(2,2′-dichloro-3′-(2-((((R)-2-hydroxypropyl)amino)methyl)imidazo[1,2-b]pyridazin-6-yl)-[1,1′-biphenyl]-3-yl)-2-methoxypyridin-3-yl)methyl)amino)propan-2-ol;
(R)-1-(((6-(2,2′-dichloro-3′-(2-((((R)-2-hydroxypropyl)amino)methyl)imidazo[1,2-b]pyridazin-6-yl)-[1,1′-biphenyl]-3-yl)-2-methoxypyridin-3-yl)methyl)amino)propan-2-ol;
(S)-2-(((8-(2,2′-dichloro-3′-(6-methoxy-5-((((5-oxopyrrolidin-2-yl)methyl)amino)methyl)pyridin-2-yl)-[1,1′-biphenyl]-3-yl)-4-oxo-4H-pyrido[1,2-a]pyrimidin-2-yl)methyl)amino)-N,2-dimethylpropanamide;
(R)-2-(((8-(2,2′-dichloro-3′-(6-methoxy-5-((((5-oxopyrrolidin-2-yl)methyl)amino)methyl)pyridin-2-yl)-[1,1′-biphenyl]-3-yl)-4-oxo-4H-pyrido[1,2-a]pyrimidin-2-yl)methyl)amino)-N,2-dimethylpropanamide;
8-(2,2′-dichloro-3′-(6-methoxy-5-(((((S)-5-oxopyrrolidin-2-yl)methyl)amino)methyl)pyridin-2-yl)-[1,1′-biphenyl]-3-yl)-2-(((S)-4-hydroxy-2-oxopyrrolidin-1-yl)methyl)-4H-pyrido[1,2-a]pyrimidin-4-one;
8-(2,2′-dichloro-3′-(6-methoxy-5-(((((R)-5-oxopyrrolidin-2-yl)methyl)amino)methyl)pyridin-2-yl)-[1,1′-biphenyl]-3-yl)-2-(((S)-4-hydroxy-2-oxopyrrolidin-1-yl)methyl)-4H-pyrido[1,2-a]pyrimidin-4-one;
8-(2,2′-dichloro-3′-(6-methoxy-5-(((((S)-5-oxopyrrolidin-2-yl)methyl)amino)methyl)pyridin-2-yl)-[1,1′-biphenyl]-3-yl)-2-(((R)-4-hydroxy-2-oxopyrrolidin-1-yl)methyl)-4H-pyrido[1,2-a]pyrimidin-4-one;
8-(2,2′-dichloro-3′-(6-methoxy-5-(((((R)-5-oxopyrrolidin-2-yl)methyl)amino)methyl)pyridin-2-yl)-[1,1′-biphenyl]-3-yl)-2-(((R)-4-hydroxy-2-oxopyrrolidin-1-yl)methyl)-4H-pyrido[1,2-a]pyrimidin-4-one;
methyl (S)-1-((8-(2,2′-dichloro-3′-(6-methoxy-5-((((5-oxopyrrolidin-2-yl)methyl)amino)methyl)pyridin-2-yl)-[1,1′-biphenyl]-3-yl)-4-oxo-4H-pyrido[1,2-a]pyrimidin-2-yl)methyl)azetidine-3-carboxylate;
methyl (R)-1-((8-(2,2′-dichloro-3′-(6-methoxy-5-((((5-oxopyrrolidin-2-yl)methyl)amino)methyl)pyridin-2-yl)-[1,1′-biphenyl]-3-yl)-4-oxo-4H-pyrido[1,2-a]pyrimidin-2-yl)methyl)azetidine-3-carboxylate;
(S)-5-((((6-(2,2′-dichloro-3′-(2,3,4,5-tetrahydro-1H-benzo[c]azepin-7-yl)-[1,1′-biphenyl]-3-yl)-2-methoxypyridin-3-yl)methyl)amino)methyl)pyrrolidin-2-one;
(R)-5-((((6-(2,2′-dichloro-3′-(2,3,4,5-tetrahydro-1H-benzo[c]azepin-7-yl)-[1,1′-biphenyl]-3-yl)-2-methoxypyridin-3-yl)methyl)amino)methyl)pyrrolidin-2-one;
(S)-8-(2,2′-dichloro-3′-(6-methoxy-5-((((5-oxopyrrolidin-2-yl)methyl)amino)methyl)pyridin-2-yl)-[1,1′-biphenyl]-3-yl)-3-(((2-methoxy-2-methylpropyl)amino)methyl)-4H-pyrido[1,2-a]pyrimidin-4-one;
(R)-8-(2,2′-dichloro-3′-(6-methoxy-5-((((5-oxopyrrolidin-2-yl)methyl)amino)methyl)pyridin-2-yl)-[1,1′-biphenyl]-3-yl)-3-(((2-methoxy-2-methylpropyl)amino)methyl)-4H-pyrido[1,2-a]pyrimidin-4-one;
(S)-8-(2,2′-dichloro-3′-(6-methoxy-5-((((5-oxopyrrolidin-2-yl)methyl)amino)methyl)pyridin-2-yl)-[1,1′-biphenyl]-3-yl)-3-((6-hydroxy-2-azaspiro[3.3]heptan-2-yl)methyl)-4H-pyrido[1,2-a]pyrimidin-4-one;
(R)-8-(2,2′-dichloro-3′-(6-methoxy-5-((((5-oxopyrrolidin-2-yl)methyl)amino)methyl)pyridin-2-yl)-[1,1′-biphenyl]-3-yl)-3-((6-hydroxy-2-azaspiro[3.3]heptan-2-yl)methyl)-4H-pyrido[1,2-a]pyrimidin-4-one;
8-(2,2′-dichloro-3′-(6-methoxy-5-(((((S)-5-oxopyrrolidin-2-yl)methyl)amino)methyl)pyridin-2-yl)-[1,1′-biphenyl]-3-yl)-3-(((3R,4R)-3,4-dihydroxypyrrolidin-1-yl)methyl)-4H-pyrido[1,2-a]pyrimidin-4-one;
8-(2,2′-dichloro-3′-(6-methoxy-5-(((((S)-5-oxopyrrolidin-2-yl)methyl)amino)methyl)pyridin-2-yl)-[1,1′-biphenyl]-3-yl)-3-(((3R,4S)-3,4-dihydroxypyrrolidin-1-yl)methyl)-4H-pyrido[1,2-a]pyrimidin-4-one;
8-(2,2′-dichloro-3′-(6-methoxy-5-(((((S)-5-oxopyrrolidin-2-yl)methyl)amino)methyl)pyridin-2-yl)-[1,1′-biphenyl]-3-yl)-3-(((3S,4R)-3,4-dihydroxypyrrolidin-1-yl)methyl)-4H-pyrido[1,2-a]pyrimidin-4-one;
8-(2,2′-dichloro-3′-(6-methoxy-5-(((((S)-5-oxopyrrolidin-2-yl)methyl)amino)methyl)pyridin-2-yl)-[1,1′-biphenyl]-3-yl)-3-(((3S,4S)-3,4-dihydroxypyrrolidin-1-yl)methyl)-4H-pyrido[1,2-a]pyrimidin-4-one;
8-(2,2′-dichloro-3′-(6-methoxy-5-(((((R)-5-oxopyrrolidin-2-yl)methyl)amino)methyl)pyridin-2-yl)-[1,1′-biphenyl]-3-yl)-3-(((3R,4R)-3,4-dihydroxypyrrolidin-1-yl)methyl)-4H-pyrido[1,2-a]pyrimidin-4-one;
8-(2,2′-dichloro-3′-(6-methoxy-5-(((((R)-5-oxopyrrolidin-2-yl)methyl)amino)methyl)pyridin-2-yl)-[1,1′-biphenyl]-3-yl)-3-(((3R,4S)-3,4-dihydroxypyrrolidin-1-yl)methyl)-4H-pyrido[1,2-a]pyrimidin-4-one;
8-(2,2′-dichloro-3′-(6-methoxy-5-(((((R)-5-oxopyrrolidin-2-yl)methyl)amino)methyl)pyridin-2-yl)-[1,1′-biphenyl]-3-yl)-3-(((3S,4R)-3,4-dihydroxypyrrolidin-1-yl)methyl)-4H-pyrido[1,2-a]pyrimidin-4-one;
8-(2,2′-dichloro-3′-(6-methoxy-5-(((((R)-5-oxopyrrolidin-2-yl)methyl)amino)methyl)pyridin-2-yl)-[1,1′-biphenyl]-3-yl)-3-(((3S,4S)-3,4-dihydroxypyrrolidin-1-yl)methyl)-4H-pyrido[1,2-a]pyrimidin-4-one;
8-(2,2′-dichloro-3′-(6-methoxy-5-(((((R)-5-oxopyrrolidin-2-yl)methyl)amino)methyl)pyridin-2-yl)-[1,1′-biphenyl]-3-yl)-3-((((1r,3r)-3-hydroxy-3-methylcyclobutyl)amino)methyl)-2-methoxy-4H-pyrido[1,2-a]pyrimidin-4-one;
8-(2,2′-dichloro-3′-(6-methoxy-5-(((((S)-5-oxopyrrolidin-2-yl)methyl)amino)methyl)pyridin-2-yl)-[1,1′-biphenyl]-3-yl)-3-((((1s,3s)-3-hydroxy-3-methylcyclobutyl)amino)methyl)-2-methoxy-4H-pyrido[1,2-a]pyrimidin-4-one;
8-(2,2′-dichloro-3′-(6-methoxy-5-(((((R)-5-oxopyrrolidin-2-yl)methyl)amino)methyl)pyridin-2-yl)-[1,1′-biphenyl]-3-yl)-3-((((1s,3s)-3-hydroxy-3-methylcyclobutyl)amino)methyl)-2-methoxy-4H-pyrido[1,2-a]pyrimidin-4-one;
8-(2,2′-dichloro-3′-(6-methoxy-5-(((((S)-5-oxopyrrolidin-2-yl)methyl)amino)methyl)pyridin-2-yl)-[1,1′-biphenyl]-3-yl)-3-((((1r,3r)-3-hydroxy-3-methylcyclobutyl)amino)methyl)-2-methoxy-4H-pyrido[1,2-a]pyrimidin-4-one;
(S)-1-(((8-(2,2′-dichloro-3′-(6-methoxy-5-((((5-oxopyrrolidin-2-yl)methyl)amino)methyl)pyridin-2-yl)-[1,1′-biphenyl]-3-yl)-4-oxo-4H-pyrido[1,2-a]pyrimidin-2-yl)methyl)amino)-N-methylcyclopropane-1-carboxamide;
(R)-1-(((8-(2,2′-dichloro-3′-(6-methoxy-5-((((5-oxopyrrolidin-2-yl)methyl)amino)methyl)pyridin-2-yl)-[1,1′-biphenyl]-3-yl)-4-oxo-4H-pyrido[1,2-a]pyrimidin-2-yl)methyl)amino)-N-methylcyclopropane-1-carboxamide;
6-(2,2′-dichloro-3′-(6-methoxy-5-(((((S)-5-oxopyrrolidin-2-yl)methyl)amino)methyl)pyridin-2-yl)-[1,1′-biphenyl]-3-yl)-2-((((S)-2-hydroxypropyl)amino)methyl)-3-methylpyrrolo[2,1-f][1,2,4]triazin-4(3H)-one;
6-(2,2′-dichloro-3′-(6-methoxy-5-(((((S)-5-oxopyrrolidin-2-yl)methyl)amino)methyl)pyridin-2-yl)-[1,1′-biphenyl]-3-yl)-2-((((R)-2-hydroxypropyl)amino)methyl)-3-methylpyrrolo[2,1-f][1,2,4]triazin-4(3H)-one;
6-(2,2′-dichloro-3′-(6-methoxy-5-(((((R)-5-oxopyrrolidin-2-yl)methyl)amino)methyl)pyridin-2-yl)-[1,1′-biphenyl]-3-yl)-2-((((S)-2-hydroxypropyl)amino)methyl)-3-methylpyrrolo[2,1-f][1,2,4]triazin-4(3H)-one;
6-(2,2′-dichloro-3′-(6-methoxy-5-(((((R)-5-oxopyrrolidin-2-yl)methyl)amino)methyl)pyridin-2-yl)-[1,1′-biphenyl]-3-yl)-2-((((R)-2-hydroxypropyl)amino)methyl)-3-methylpyrrolo[2,1-f][1,2,4]triazin-4(3H)-one;
(S)-6-(2,2′-dichloro-3′-(6-methoxy-5-((((5-oxopyrrolidin-2-yl)methyl)amino)methyl)pyridin-2-yl)-[1,1′-biphenyl]-3-yl)-2-(((2-hydroxyethyl)amino)methyl)-3-methylpyrrolo[2,1-f][1,2,4]triazin-4(3H)-one;
(R)-6-(2,2′-dichloro-3′-(6-methoxy-5-((((5-oxopyrrolidin-2-yl)methyl)amino)methyl)pyridin-2-yl)-[1,1′-biphenyl]-3-yl)-2-(((2-hydroxyethyl)amino)methyl)-3-methylpyrrolo[2,1-f][1,2,4]triazin-4(3H)-one;
(S)-5-((((6-(2,2′-dichloro-3′-(2-(((2-hydroxyethyl)amino)methyl)imidazo[1,2-b]pyridazin-6-yl)-[1,1′-biphenyl]-3-yl)-2-methoxypyridin-3-yl)methyl)amino)methyl)pyrrolidin-2-one;
(R)-5-((((6-(2,2′-dichloro-3′-(2-(((2-hydroxyethyl)amino)methyl)imidazo[1,2-b]pyridazin-6-yl)-[1,1′-biphenyl]-3-yl)-2-methoxypyridin-3-yl)methyl)amino)methyl)pyrrolidin-2-one;
(S)-5-((((6-(2,2′-dichloro-3′-(2-(((2-hydroxyethyl)(methyl)amino)methyl)imidazo[1,2-b]pyridazin-6-yl)-[1,1′-biphenyl]-3-yl)-2-methoxypyridin-3-yl)methyl)amino)methyl)pyrrolidin-2-one;
(R)-5-((((6-(2,2′-dichloro-3′-(2-(((2-hydroxyethyl)(methyl)amino)methyl)imidazo[1,2-b]pyridazin-6-yl)-[1,1′-biphenyl]-3-yl)-2-methoxypyridin-3-yl)methyl)amino)methyl)pyrrolidin-2-one;
(S)-5-((((6-(2,2′-dichloro-3′-(2-((((S)-2-hydroxypropyl)amino)methyl)imidazo[1,2-b]pyridazin-6-yl)-[1,1′-biphenyl]-3-yl)-2-methoxypyridin-3-yl)methyl)amino)methyl)pyrrolidin-2-one;
(R)-5-((((6-(2,2′-dichloro-3′-(2-((((S)-2-hydroxypropyl)amino)methyl)imidazo[1,2-b]pyridazin-6-yl)-[1,1′-biphenyl]-3-yl)-2-methoxypyridin-3-yl)methyl)amino)methyl)pyrrolidin-2-one;
(S)-5-((((6-(2,2′-dichloro-3′-(2-((((R)-2-hydroxypropyl)amino)methyl)imidazo[1,2-b]pyridazin-6-yl)-[1,1′-biphenyl]-3-yl)-2-methoxypyridin-3-yl)methyl)amino)methyl)pyrrolidin-2-one;
(R)-5-((((6-(2,2′-dichloro-3′-(2-((((R)-2-hydroxypropyl)amino)methyl)imidazo[1,2-b]pyridazin-6-yl)-[1,1′-biphenyl]-3-yl)-2-methoxypyridin-3-yl)methyl)amino)methyl)pyrrolidin-2-one;
(S)-4-(((6-(2,2′-dichloro-3′-(6-methoxy-5-(((((S)-5-oxopyrrolidin-2-yl)methyl)amino)methyl)pyridin-2-yl)-[1,1′-biphenyl]-3-yl)imidazo[1,2-b]pyridazin-2-yl)methyl)amino)-3-hydroxy-N,N-dimethylbutanamide;
(R)-4-(((6-(2,2′-dichloro-3′-(6-methoxy-5-(((((S)-5-oxopyrrolidin-2-yl)methyl)amino)methyl)pyridin-2-yl)-[1,1′-biphenyl]-3-yl)imidazo[1,2-b]pyridazin-2-yl)methyl)amino)-3-hydroxy-N,N-dimethylbutanamide;
(S)-4-(((6-(2,2′-dichloro-3′-(6-methoxy-5-(((((R)-5-oxopyrrolidin-2-yl)methyl)amino)methyl)pyridin-2-yl)-[1,1′-biphenyl]-3-yl)imidazo[1,2-b]pyridazin-2-yl)methyl)amino)-3-hydroxy-N,N-dimethylbutanamide;
(R)-4-(((6-(2,2′-dichloro-3′-(6-methoxy-5-(((((R)-5-oxopyrrolidin-2-yl)methyl)amino)methyl)pyridin-2-yl)-[1,1′-biphenyl]-3-yl)imidazo[1,2-b]pyridazin-2-yl)methyl)amino)-3-hydroxy-N,N-dimethylbutanamide;
(S)-5-((((6-(2,2′-dichloro-3′-(2-(((((S)-5-oxopyrrolidin-2-yl)methyl)amino)methyl)imidazo[1,2-a]pyridin-6-yl)-[1,1′-biphenyl]-3-yl)-2-methoxypyridin-3-yl)methyl)amino)methyl)pyrrolidin-2-one;
(R)-5-((((6-(2,2′-dichloro-3′-(2-(((((S)-5-oxopyrrolidin-2-yl)methyl)amino)methyl)imidazo[1,2-a]pyridin-6-yl)-[1,1′-biphenyl]-3-yl)-2-methoxypyridin-3-yl)methyl)amino)methyl)pyrrolidin-2-one;
(S)-5-((((6-(2,2′-dichloro-3′-(2-(((((R)-5-oxopyrrolidin-2-yl)methyl)amino)methyl)imidazo[1,2-a]pyridin-6-yl)-[1,1′-biphenyl]-3-yl)-2-methoxypyridin-3-yl)methyl)amino)methyl)pyrrolidin-2-one;
(R)-5-((((6-(2,2′-dichloro-3′-(2-(((((R)-5-oxopyrrolidin-2-yl)methyl)amino)methyl)imidazo[1,2-a]pyridin-6-yl)-[1,1′-biphenyl]-3-yl)-2-methoxypyridin-3-yl)methyl)amino)methyl)pyrrolidin-2-one;
(S)-5-((((6-(2,2′-dichloro-3′-(2-(((((S)-5-oxopyrrolidin-2-yl)methyl)amino)methyl)imidazo[1,2-a]pyridin-7-yl)-[1,1′-biphenyl]-3-yl)-2-methoxypyridin-3-yl)methyl)amino)methyl)pyrrolidin-2-one;
(R)-5-((((6-(2,2′-dichloro-3′-(2-(((((S)-5-oxopyrrolidin-2-yl)methyl)amino)methyl)imidazo[1,2-a]pyridin-7-yl)-[1,1′-biphenyl]-3-yl)-2-methoxypyridin-3-yl)methyl)amino)methyl)pyrrolidin-2-one;
(S)-5-((((6-(2,2′-dichloro-3′-(2-(((((R)-5-oxopyrrolidin-2-yl)methyl)amino)methyl)imidazo[1,2-a]pyridin-7-yl)-[1,1′-biphenyl]-3-yl)-2-methoxypyridin-3-yl)methyl)amino)methyl)pyrrolidin-2-one;
(R)-5-((((6-(2,2′-dichloro-3′-(2-(((((R)-5-oxopyrrolidin-2-yl)methyl)amino)methyl)imidazo[1,2-a]pyridin-7-yl)-[1,1′-biphenyl]-3-yl)-2-methoxypyridin-3-yl)methyl)amino)methyl)pyrrolidin-2-one;
methyl (S)-3-((((8-(2,2′-dichloro-3′-(6-methoxy-5-((((5-oxopyrrolidin-2-yl)methyl)amino)methyl)pyridin-2-yl)-[1,1′-biphenyl]-3-yl)-4-oxo-4H-pyrido[1,2-a]pyrimidin-3-yl)methyl)amino)methyl)oxetane-3-carboxylate;
methyl (R)-3-((((8-(2,2′-dichloro-3′-(6-methoxy-5-((((5-oxopyrrolidin-2-yl)methyl)amino)methyl)pyridin-2-yl)-[1,1′-biphenyl]-3-yl)-4-oxo-4H-pyrido[1,2-a]pyrimidin-3-yl)methyl)amino)methyl)oxetane-3-carboxylate;
8-(2,2′-dichloro-3′-(6-methoxy-5-(((((S)-5-oxopyrrolidin-2-yl)methyl)amino)methyl)pyridin-2-yl)-[1,1′-biphenyl]-3-yl)-3-(((((R)-3,3-dimethyl-5-oxopyrrolidin-2-yl)methyl)amino)methyl)-4H-pyrido[1,2-a]pyrimidin-4-one;
8-(2,2′-dichloro-3′-(6-methoxy-5-(((((S)-5-oxopyrrolidin-2-yl)methyl)amino)methyl)pyridin-2-yl)-[1,1′-biphenyl]-3-yl)-3-(((((S)-3,3-dimethyl-5-oxopyrrolidin-2-yl)methyl)amino)methyl)-4H-pyrido[1,2-a]pyrimidin-4-one;
8-(2,2′-dichloro-3′-(6-methoxy-5-(((((R)-5-oxopyrrolidin-2-yl)methyl)amino)methyl)pyridin-2-yl)-[1,1′-biphenyl]-3-yl)-3-(((((R)-3,3-dimethyl-5-oxopyrrolidin-2-yl)methyl)amino)methyl)-4H-pyrido[1,2-a]pyrimidin-4-one;
8-(2,2′-dichloro-3′-(6-methoxy-5-(((((R)-5-oxopyrrolidin-2-yl)methyl)amino)methyl)pyridin-2-yl)-[1,1′-biphenyl]-3-yl)-3-(((((S)-3,3-dimethyl-5-oxopyrrolidin-2-yl)methyl)amino)methyl)-4H-pyrido[1,2-a]pyrimidin-4-one;
8-(2,2′-dichloro-3′-(5-methoxy-6-(((((S)-5-oxopyrrolidin-2-yl)methyl)amino)methyl)pyridin-3-yl)-[1,1′-biphenyl]-3-yl)-3-(((((S)-5-oxopyrrolidin-2-yl)methyl)amino)methyl)-4H-pyrido[1,2-a]pyrimidin-4-one;
8-(2,2′-dichloro-3′-(5-methoxy-6-(((((R)-5-oxopyrrolidin-2-yl)methyl)amino)methyl)pyridin-3-yl)-[1,1′-biphenyl]-3-yl)-3-(((((S)-5-oxopyrrolidin-2-yl)methyl)amino)methyl)-4H-pyrido[1,2-a]pyrimidin-4-one;
8-(2,2′-dichloro-3′-(5-methoxy-6-(((((S)-5-oxopyrrolidin-2-yl)methyl)amino)methyl)pyridin-3-yl)-[1,1′-biphenyl]-3-yl)-3-(((((R)-5-oxopyrrolidin-2-yl)methyl)amino)methyl)-4H-pyrido[1,2-a]pyrimidin-4-one;
8-(2,2′-dichloro-3′-(5-methoxy-6-(((((R)-5-oxopyrrolidin-2-yl)methyl)amino)methyl)pyridin-3-yl)-[1,1′-biphenyl]-3-yl)-3-(((((R)-5-oxopyrrolidin-2-yl)methyl)amino)methyl)-4H-pyrido[1,2-a]pyrimidin-4-one;
8-(2,2′-dichloro-3′-(6-((((1r,3r)-3-hydroxy-3-methylcyclobutyl)amino)methyl)-5-methoxypyridin-3-yl)-[1,1′-biphenyl]-3-yl)-3-((((1r,3r)-3-hydroxy-3-methylcyclobutyl)amino)methyl)-4H-pyrido[1,2-a]pyrimidin-4-one;
8-(2,2′-dichloro-3′-(6-((((1r,3r)-3-hydroxy-3-methylcyclobutyl)amino)methyl)-5-methoxypyridin-3-yl)-[1,1′-biphenyl]-3-yl)-3-((((1s,3s)-3-hydroxy-3-methylcyclobutyl)amino)methyl)-4H-pyrido[1,2-a]pyrimidin-4-one;
8-(2,2′-dichloro-3′-(6-((((1s,3s)-3-hydroxy-3-methylcyclobutyl)amino)methyl)-5-methoxypyridin-3-yl)-[1,1′-biphenyl]-3-yl)-3-((((1r,3r)-3-hydroxy-3-methylcyclobutyl)amino)methyl)-4H-pyrido[1,2-a]pyrimidin-4-one;
8-(2,2′-dichloro-3′-(6-((((1s,3s)-3-hydroxy-3-methylcyclobutyl)amino)methyl)-5-methoxypyridin-3-yl)-[1,1′-biphenyl]-3-yl)-3-((((1s,3s)-3-hydroxy-3-methylcyclobutyl)amino)methyl)-4H-pyrido[1,2-a]pyrimidin-4-one;
8-(2,2′-dichloro-3′-(6-(((2-hydroxyethyl)(methyl)amino)methyl)-5-methoxypyridin-3-yl)-[1,1′-biphenyl]-3-yl)-3-(((2-hydroxyethyl)(methyl)amino)methyl)-4H-pyrido[1,2-a]pyrimidin-4-one;
(S)-5-((((6-(2,2′-dichloro-3′-(1-methyl-2,3,4,5-tetrahydro-1H-benzo[e][1,4]diazepin-7-yl)-[1,1′-biphenyl]-3-yl)-2-methoxypyridin-3-yl)methyl)amino)methyl)pyrrolidin-2-one;
(R)-5-((((6-(2,2′-dichloro-3′-(1-methyl-2,3,4,5-tetrahydro-1H-benzo[e][1,4]diazepin-7-yl)-[1,1′-biphenyl]-3-yl)-2-methoxypyridin-3-yl)methyl)amino)methyl)pyrrolidin-2-one;
(S)-5-((((6-(2,2′-dichloro-3′-(1-methyl-2,3,4,5-tetrahydro-1H-benzo[e][1,4]diazepin-8-yl)-[1,1′-biphenyl]-3-yl)-2-methoxypyridin-3-yl)methyl)amino)methyl)pyrrolidin-2-one;
(R)-5-((((6-(2,2′-dichloro-3′-(1-methyl-2,3,4,5-tetrahydro-1H-benzo[e][1,4]diazepin-8-yl)-[1,1′-biphenyl]-3-yl)-2-methoxypyridin-3-yl)methyl)amino)methyl)pyrrolidin-2-one;
(S)-5-((((6-(2,2′-dichloro-3′-(1,2,3,4-tetrahydropyrazino[1,2-a]indol-8-yl)-[1,1′-biphenyl]-3-yl)-2-methoxypyridin-3-yl)methyl)amino)methyl)pyrrolidin-2-one;
(R)-5-((((6-(2,2′-dichloro-3′-(1,2,3,4-tetrahydropyrazino[1,2-a]indol-8-yl)-[1,1′-biphenyl]-3-yl)-2-methoxypyridin-3-yl)methyl)amino)methyl)pyrrolidin-2-one;
(S)-5-((((3-chloro-5-(2,2′-dichloro-3′-(6-methoxy-5-(((((S)-5-oxopyrrolidin-2-yl)methyl)amino)methyl)pyridin-2-yl)-[1,1′-biphenyl]-3-yl)-1-methyl-1H-pyrrolo[3,2-b]pyridin-2-yl)methyl)amino)methyl)pyrrolidin-2-one;
(R)-5-((((3-chloro-5-(2,2′-dichloro-3′-(6-methoxy-5-(((((S)-5-oxopyrrolidin-2-yl)methyl)amino)methyl)pyridin-2-yl)-[1,1′-biphenyl]-3-yl)-1-methyl-1H-pyrrolo[3,2-b]pyridin-2-yl)methyl)amino)methyl)pyrrolidin-2-one;
(S)-5-((((3-chloro-5-(2,2′-dichloro-3′-(6-methoxy-5-(((((R)-5-oxopyrrolidin-2-yl)methyl)amino)methyl)pyridin-2-yl)-[1,1′-biphenyl]-3-yl)-1-methyl-1H-pyrrolo[3,2-b]pyridin-2-yl)methyl)amino)methyl)pyrrolidin-2-one;
(R)-5-((((3-chloro-5-(2,2′-dichloro-3′-(6-methoxy-5-(((((R)-5-oxopyrrolidin-2-yl)methyl)amino)methyl)pyridin-2-yl)-[1,1′-biphenyl]-3-yl)-1-methyl-1H-pyrrolo[3,2-b]pyridin-2-yl)methyl)amino)methyl)pyrrolidin-2-one;
(S)-5-((((6-(2,2′-dichloro-3′-(8-chloro-6-((((R)-2-hydroxypropyl)amino)methyl)-5-methoxyquinolin-2-yl)-[1,1′-biphenyl]-3-yl)-2-methoxypyridin-3-yl)methyl)amino)methyl)pyrrolidin-2-one;
(R)-5-((((6-(2,2′-dichloro-3′-(8-chloro-6-((((R)-2-hydroxypropyl)amino)methyl)-5-methoxyquinolin-2-yl)-[1,1′-biphenyl]-3-yl)-2-methoxypyridin-3-yl)methyl)amino)methyl)pyrrolidin-2-one;
(S)-5-((((6-(2,2′-dichloro-3′-(8-chloro-6-((((S)-2-hydroxypropyl)amino)methyl)-5-methoxyquinolin-2-yl)-[1,1′-biphenyl]-3-yl)-2-methoxypyridin-3-yl)methyl)amino)methyl)pyrrolidin-2-one;
(R)-5-((((6-(2,2′-dichloro-3′-(8-chloro-6-((((S)-2-hydroxypropyl)amino)methyl)-5-methoxyquinolin-2-yl)-[1,1′-biphenyl]-3-yl)-2-methoxypyridin-3-yl)methyl)amino)methyl)pyrrolidin-2-one;
8-(2,2′-dichloro-3′-(6-methoxy-5-(((((S)-5-oxopyrrolidin-2-yl)methyl)amino)methyl)pyridin-2-yl)-[1,1′-biphenyl]-3-yl)-3-(((((2R)-methyl-5-oxopyrrolidin-2-yl)methyl)amino)methyl)-4H-pyrido[1,2-a]pyrimidin-4-one;
8-(2,2′-dichloro-3′-(6-methoxy-5-(((((S)-5-oxopyrrolidin-2-yl)methyl)amino)methyl)pyridin-2-yl)-[1,1′-biphenyl]-3-yl)-3-(((((2S)-methyl-5-oxopyrrolidin-2-yl)methyl)amino)methyl)-4H-pyrido[1,2-a]pyrimidin-4-one;
8-(2,2′-dichloro-3′-(6-methoxy-5-(((((R)-5-oxopyrrolidin-2-yl)methyl)amino)methyl)pyridin-2-yl)-[1,1′-biphenyl]-3-yl)-3-(((((2R)-methyl-5-oxopyrrolidin-2-yl)methyl)amino)methyl)-4H-pyrido[1,2-a]pyrimidin-4-one;
8-(2,2′-dichloro-3′-(6-methoxy-5-(((((R)-5-oxopyrrolidin-2-yl)methyl)amino)methyl)pyridin-2-yl)-[1,1′-biphenyl]-3-yl)-3-(((((2S)-methyl-5-oxopyrrolidin-2-yl)methyl)amino)methyl)-4H-pyrido[1,2-a]pyrimidin-4-one;
(S)-5-((((6-(2,2′-dichloro-3′-(6-(((((S)-5-oxopyrrolidin-2-yl)methyl)amino)methyl)quinolin-2-yl)-[1,1′-biphenyl]-3-yl)-2-methoxypyridin-3-yl)methyl)amino)methyl)pyrrolidin-2-one;
(R)-5-((((6-(2,2′-dichloro-3′-(6-(((((S)-5-oxopyrrolidin-2-yl)methyl)amino)methyl)quinolin-2-yl)-[1,1′-biphenyl]-3-yl)-2-methoxypyridin-3-yl)methyl)amino)methyl)pyrrolidin-2-one;
(S)-5-((((6-(2,2′-dichloro-3′-(6-(((((R)-5-oxopyrrolidin-2-yl)methyl)amino)methyl)quinolin-2-yl)-[1,1′-biphenyl]-3-yl)-2-methoxypyridin-3-yl)methyl)amino)methyl)pyrrolidin-2-one;
(R)-5-((((6-(2,2′-dichloro-3′-(6-(((((R)-5-oxopyrrolidin-2-yl)methyl)amino)methyl)quinolin-2-yl)-[1,1′-biphenyl]-3-yl)-2-methoxypyridin-3-yl)methyl)amino)methyl)pyrrolidin-2-one;
(S)-5-((((3-(2,2′-dichloro-3′-(6-methoxy-5-(((((S)-5-oxopyrrolidin-2-yl)methyl)amino)methyl)pyridin-2-yl)-[1,1′-biphenyl]-3-yl)quinolin-7-yl)methyl)amino)methyl)pyrrolidin-2-one;
(R)-5-((((3-(2,2′-dichloro-3′-(6-methoxy-5-(((((S)-5-oxopyrrolidin-2-yl)methyl)amino)methyl)pyridin-2-yl)-[1,1′-biphenyl]-3-yl)quinolin-7-yl)methyl)amino)methyl)pyrrolidin-2-one;
(S)-5-((((3-(2,2′-dichloro-3′-(6-methoxy-5-(((((R)-5-oxopyrrolidin-2-yl)methyl)amino)methyl)pyridin-2-yl)-[1,1′-biphenyl]-3-yl)quinolin-7-yl)methyl)amino)methyl)pyrrolidin-2-one;
(R)-5-((((3-(2,2′-dichloro-3′-(6-methoxy-5-(((((R)-5-oxopyrrolidin-2-yl)methyl)amino)methyl)pyridin-2-yl)-[1,1′-biphenyl]-3-yl)quinolin-7-yl)methyl)amino)methyl)pyrrolidin-2-one;
(S)-8-(2,2′-dichloro-3′-(6-methoxy-5-((((5-oxopyrrolidin-2-yl)methyl)amino)methyl)pyridin-2-yl)-[1,1′-biphenyl]-3-yl)-2-(((2-hydroxyethyl)amino)methyl)-3-methyl-4H-pyrido[1,2-a]pyrimidin-4-one;
(R)-8-(2,2′-dichloro-3′-(6-methoxy-5-((((5-oxopyrrolidin-2-yl)methyl)amino)methyl)pyridin-2-yl)-[1,1′-biphenyl]-3-yl)-2-(((2-hydroxyethyl)amino)methyl)-3-methyl-4H-pyrido[1,2-a]pyrimidin-4-one;
8-(2,2′-dichloro-3′-(6-methoxy-5-(((((S)-5-oxopyrrolidin-2-yl)methyl)amino)methyl)pyridin-2-yl)-[1,1′-biphenyl]-3-yl)-3-methyl-2-(((((S)-5-oxopyrrolidin-2-yl)methyl)amino)methyl)-4H-pyrido[1,2-a]pyrimidin-4-one;
8-(2,2′-dichloro-3′-(6-methoxy-5-(((((R)-5-oxopyrrolidin-2-yl)methyl)amino)methyl)pyridin-2-yl)-[1,1′-biphenyl]-3-yl)-3-methyl-2-(((((S)-5-oxopyrrolidin-2-yl)methyl)amino)methyl)-4H-pyrido[1,2-a]pyrimidin-4-one;
8-(2,2′-dichloro-3′-(6-methoxy-5-(((((S)-5-oxopyrrolidin-2-yl)methyl)amino)methyl)pyridin-2-yl)-[1,1′-biphenyl]-3-yl)-3-methyl-2-(((((R)-5-oxopyrrolidin-2-yl)methyl)amino)methyl)-4H-pyrido[1,2-a]pyrimidin-4-one;
8-(2,2′-dichloro-3′-(6-methoxy-5-(((((R)-5-oxopyrrolidin-2-yl)methyl)amino)methyl)pyridin-2-yl)-[1,1′-biphenyl]-3-yl)-3-methyl-2-(((((R)-5-oxopyrrolidin-2-yl)methyl)amino)methyl)-4H-pyrido[1,2-a]pyrimidin-4-one;
8-(2,2′-dichloro-3′-(6-methoxy-5-(((((S)-5-oxopyrrolidin-2-yl)methyl)amino)methyl)pyridin-2-yl)-[1,1′-biphenyl]-3-yl)-2-((((1r,3r)-3-hydroxy-3-methylcyclobutyl)amino)methyl)-3-methyl-4H-pyrido[1,2-a]pyrimidin-4-one;
8-(2,2′-dichloro-3′-(6-methoxy-5-(((((S)-5-oxopyrrolidin-2-yl)methyl)amino)methyl)pyridin-2-yl)-[1,1′-biphenyl]-3-yl)-2-((((1s,3s)-3-hydroxy-3-methylcyclobutyl)amino)methyl)-3-methyl-4H-pyrido[1,2-a]pyrimidin-4-one;
8-(2,2′-dichloro-3′-(6-methoxy-5-(((((R)-5-oxopyrrolidin-2-yl)methyl)amino)methyl)pyridin-2-yl)-[1,1′-biphenyl]-3-yl)-2-((((1r,3r)-3-hydroxy-3-methylcyclobutyl)amino)methyl)-3-methyl-4H-pyrido[1,2-a]pyrimidin-4-one;
8-(2,2′-dichloro-3′-(6-methoxy-5-(((((R)-5-oxopyrrolidin-2-yl)methyl)amino)methyl)pyridin-2-yl)-[1,1′-biphenyl]-3-yl)-2-((((1s,3s)-3-hydroxy-3-methylcyclobutyl)amino)methyl)-3-methyl-4H-pyrido[1,2-a]pyrimidin-4-one;
(S)-10-(2,2′-dichloro-3′-(6-methoxy-5-((((5-oxopyrrolidin-2-yl)methyl)amino)methyl)pyridin-2-yl)-[1,1′-biphenyl]-3-yl)-1-methyl-2,3,4,5-tetrahydropyrido[1′,2′:1,2]pyrimido[4,5-e][1,4]diazepin-6(1H)-one;
(R)-10-(2,2′-dichloro-3′-(6-methoxy-5-((((5-oxopyrrolidin-2-yl)methyl)amino)methyl)pyridin-2-yl)-[1,1′-biphenyl]-3-yl)-1-methyl-2,3,4,5-tetrahydropyrido[1′,2′:1,2]pyrimido[4,5-e][1,4]diazepin-6(1H)-one;
(S)-5-((((6-(2,2′-dichloro-3′-(1-methyl-3-(((((S)-5-oxopyrrolidin-2-yl)methyl)amino)methyl)-1H-pyrrolo[3,2-b]pyridin-6-yl)-[1,1′-biphenyl]-3-yl)-2-methoxypyridin-3-yl)methyl)amino)methyl)pyrrolidin-2-one;
(R)-5-((((6-(2,2′-dichloro-3′-(1-methyl-3-(((((S)-5-oxopyrrolidin-2-yl)methyl)amino)methyl)-1H-pyrrolo[3,2-b]pyridin-6-yl)-[1,1′-biphenyl]-3-yl)-2-methoxypyridin-3-yl)methyl)amino)methyl)pyrrolidin-2-one;
(S)-5-((((6-(2,2′-dichloro-3′-(1-methyl-3-(((((R)-5-oxopyrrolidin-2-yl)methyl)amino)methyl)-1H-pyrrolo[3,2-b]pyridin-6-yl)-[1,1′-biphenyl]-3-yl)-2-methoxypyridin-3-yl)methyl)amino)methyl)pyrrolidin-2-one;
(R)-5-((((6-(2,2′-dichloro-3′-(1-methyl-3-(((((R)-5-oxopyrrolidin-2-yl)methyl)amino)methyl)-1H-pyrrolo[3,2-b]pyridin-6-yl)-[1,1′-biphenyl]-3-yl)-2-methoxypyridin-3-yl)methyl)amino)methyl)pyrrolidin-2-one;
8-(2,2′-dichloro-3′-(6-methoxy-5-(((((S)-5-oxopyrrolidin-2-yl)methyl)amino)methyl)pyridin-2-yl)-[1,1′-biphenyl]-3-yl)-2-((((1r,3r)-3-hydroxy-3-methylcyclobutyl)amino)methyl)-4H-pyrido[1,2-a]pyrimidin-4-one;
8-(2,2′-dichloro-3′-(6-methoxy-5-(((((R)-5-oxopyrrolidin-2-yl)methyl)amino)methyl)pyridin-2-yl)-[1,1′-biphenyl]-3-yl)-2-((((1r,3r)-3-hydroxy-3-methylcyclobutyl)amino)methyl)-4H-pyrido[1,2-a]pyrimidin-4-one;
8-(2,2′-dichloro-3′-(6-methoxy-5-(((((S)-5-oxopyrrolidin-2-yl)methyl)amino)methyl)pyridin-2-yl)-[1,1′-biphenyl]-3-yl)-2-((((1s,3s)-3-hydroxy-3-methylcyclobutyl)amino)methyl)-4H-pyrido[1,2-a]pyrimidin-4-one;
8-(2,2′-dichloro-3′-(6-methoxy-5-(((((R)-5-oxopyrrolidin-2-yl)methyl)amino)methyl)pyridin-2-yl)-[1,1′-biphenyl]-3-yl)-2-((((1s,3s)-3-hydroxy-3-methylcyclobutyl)amino)methyl)-4H-pyrido[1,2-a]pyrimidin-4-one;
8-(2,2′-dichloro-3′-(6-methoxy-5-(((((S)-5-oxopyrrolidin-2-yl)methyl)amino)methyl)pyridin-2-yl)-[1,1′-biphenyl]-3-yl)-2-((((S)-2-hydroxypropyl)amino)methyl)-3-methyl-4H-pyrido[1,2-a]pyrimidin-4-one;
8-(2,2′-dichloro-3′-(6-methoxy-5-(((((R)-5-oxopyrrolidin-2-yl)methyl)amino)methyl)pyridin-2-yl)-[1,1′-biphenyl]-3-yl)-2-((((S)-2-hydroxypropyl)amino)methyl)-3-methyl-4H-pyrido[1,2-a]pyrimidin-4-one;
8-(2,2′-dichloro-3′-(6-methoxy-5-(((((S)-5-oxopyrrolidin-2-yl)methyl)amino)methyl)pyridin-2-yl)-[1,1′-biphenyl]-3-yl)-2-((((R)-2-hydroxypropyl)amino)methyl)-3-methyl-4H-pyrido[1,2-a]pyrimidin-4-one;
8-(2,2′-dichloro-3′-(6-methoxy-5-(((((R)-5-oxopyrrolidin-2-yl)methyl)amino)methyl)pyridin-2-yl)-[1,1′-biphenyl]-3-yl)-2-((((R)-2-hydroxypropyl)amino)methyl)-3-methyl-4H-pyrido[1,2-a]pyrimidin-4-one;
(S)-8-(2,2′-dichloro-3′-(4-chloro-6-methoxy-5-((((5-oxopyrrolidin-2-yl)methyl)amino)methyl)pyridin-2-yl)-[1,1′-biphenyl]-3-yl)-3-(((2-hydroxyethyl)amino)methyl)-4H-pyrido[1,2-a]pyrimidin-4-one;
(R)-8-(2,2′-dichloro-3′-(4-chloro-6-methoxy-5-((((5-oxopyrrolidin-2-yl)methyl)amino)methyl)pyridin-2-yl)-[1,1′-biphenyl]-3-yl)-3-(((2-hydroxyethyl)amino)methyl)-4H-pyrido[1,2-a]pyrimidin-4-one;
(S)-5-((((6-(2,2′-dichloro-3′-(2-methoxy-3-(((((S)-5-oxopyrrolidin-2-yl)methyl)amino)methyl)pyrazolo[1,5-a]pyrimidin-6-yl)-[1,1′-biphenyl]-3-yl)-2-methoxypyridin-3-yl)methyl)amino)methyl)pyrrolidin-2-one;
(R)-5-((((6-(2,2′-dichloro-3′-(2-methoxy-3-(((((S)-5-oxopyrrolidin-2-yl)methyl)amino)methyl)pyrazolo[1,5-a]pyrimidin-6-yl)-[1,1′-biphenyl]-3-yl)-2-methoxypyridin-3-yl)methyl)amino)methyl)pyrrolidin-2-one;
(S)-5-((((6-(2,2′-dichloro-3′-(2-methoxy-3-(((((R)-5-oxopyrrolidin-2-yl)methyl)amino)methyl)pyrazolo[1,5-a]pyrimidin-6-yl)-[1,1′-biphenyl]-3-yl)-2-methoxypyridin-3-yl)methyl)amino)methyl)pyrrolidin-2-one;
(R)-5-((((6-(2,2′-dichloro-3′-(2-methoxy-3-(((((R)-5-oxopyrrolidin-2-yl)methyl)amino)methyl)pyrazolo[1,5-a]pyrimidin-6-yl)-[1,1′-biphenyl]-3-yl)-2-methoxypyridin-3-yl)methyl)amino)methyl)pyrrolidin-2-one;
(S)-5-((((6-(2,2′-dichloro-3′-(3-(((((S)-5-oxopyrrolidin-2-yl)methyl)amino)methyl)imidazo[1,2-a]pyridin-7-yl)-[1,1′-biphenyl]-3-yl)-2-methoxypyridin-3-yl)methyl)amino)methyl)pyrrolidin-2-one;
(R)-5-((((6-(2,2′-dichloro-3′-(3-(((((S)-5-oxopyrrolidin-2-yl)methyl)amino)methyl)imidazo[1,2-a]pyridin-7-yl)-[1,1′-biphenyl]-3-yl)-2-methoxypyridin-3-yl)methyl)amino)methyl)pyrrolidin-2-one;
(S)-5-((((6-(2,2′-dichloro-3′-(3-(((((R)-5-oxopyrrolidin-2-yl)methyl)amino)methyl)imidazo[1,2-a]pyridin-7-yl)-[1,1′-biphenyl]-3-yl)-2-methoxypyridin-3-yl)methyl)amino)methyl)pyrrolidin-2-one;
(R)-5-((((6-(2,2′-dichloro-3′-(3-(((((R)-5-oxopyrrolidin-2-yl)methyl)amino)methyl)imidazo[1,2-a]pyridin-7-yl)-[1,1′-biphenyl]-3-yl)-2-methoxypyridin-3-yl)methyl)amino)methyl)pyrrolidin-2-one;
8-(2,2′-dichloro-3′-(3-(((((S)-5-oxopyrrolidin-2-yl)methyl)amino)methyl)pyrazolo[1,5-a]pyrimidin-6-yl)-[1,1′-biphenyl]-3-yl)-3-(((((S)-5-oxopyrrolidin-2-yl)methyl)amino)methyl)-4H-pyrido[1,2-a]pyrimidin-4-one;
8-(2,2′-dichloro-3′-(3-(((((R)-5-oxopyrrolidin-2-yl)methyl)amino)methyl)pyrazolo[1,5-a]pyrimidin-6-yl)-[1,1′-biphenyl]-3-yl)-3-(((((S)-5-oxopyrrolidin-2-yl)methyl)amino)methyl)-4H-pyrido[1,2-a]pyrimidin-4-one;
8-(2,2′-dichloro-3′-(3-(((((S)-5-oxopyrrolidin-2-yl)methyl)amino)methyl)pyrazolo[1,5-a]pyrimidin-6-yl)-[1,1′-biphenyl]-3-yl)-3-(((((R)-5-oxopyrrolidin-2-yl)methyl)amino)methyl)-4H-pyrido[1,2-a]pyrimidin-4-one;
8-(2,2′-dichloro-3′-(3-(((((R)-5-oxopyrrolidin-2-yl)methyl)amino)methyl)pyrazolo[1,5-a]pyrimidin-6-yl)-[1,1′-biphenyl]-3-yl)-3-(((((R)-5-oxopyrrolidin-2-yl)methyl)amino)methyl)-4H-pyrido[1,2-a]pyrimidin-4-one;
(S)-5-((((3-chloro-7-(2,2′-dichloro-3′-(6-methoxy-5-(((((S)-5-oxopyrrolidin-2-yl)methyl)amino)methyl)pyridin-2-yl)-[1,1′-biphenyl]-3-yl)imidazo[1,2-a]pyridin-2-yl)methyl)amino)methyl)pyrrolidin-2-one;
(R)-5-((((3-chloro-7-(2,2′-dichloro-3′-(6-methoxy-5-(((((S)-5-oxopyrrolidin-2-yl)methyl)amino)methyl)pyridin-2-yl)-[1,1′-biphenyl]-3-yl)imidazo[1,2-a]pyridin-2-yl)methyl)amino)methyl)pyrrolidin-2-one;
(S)-5-((((3-chloro-7-(2,2′-dichloro-3′-(6-methoxy-5-(((((R)-5-oxopyrrolidin-2-yl)methyl)amino)methyl)pyridin-2-yl)-[1,1′-biphenyl]-3-yl)imidazo[1,2-a]pyridin-2-yl)methyl)amino)methyl)pyrrolidin-2-one;
(R)-5-((((3-chloro-7-(2,2′-dichloro-3′-(6-methoxy-5-(((((R)-5-oxopyrrolidin-2-yl)methyl)amino)methyl)pyridin-2-yl)-[1,1′-biphenyl]-3-yl)imidazo[1,2-a]pyridin-2-yl)methyl)amino)methyl)pyrrolidin-2-one;
2-(((3-chloro-7-(2,2′-dichloro-3′-(5-(((2-hydroxyethyl)amino)methyl)-6-methoxypyridin-2-yl)-[1,1′-biphenyl]-3-yl)imidazo[1,2-a]pyridin-2-yl)methyl)amino)ethan-1-ol;
methyl (S)-3-(((8-(2,2′-dichloro-3′-(6-methoxy-5-((((5-oxopyrrolidin-2-yl)methyl)amino)methyl)pyridin-2-yl)-[1,1′-biphenyl]-3-yl)-4-oxo-4H-pyrido[1,2-a]pyrimidin-3-yl)methyl)amino)-2,2-dimethylpropanoate;
methyl (R)-3-(((8-(2,2′-dichloro-3′-(6-methoxy-5-((((5-oxopyrrolidin-2-yl)methyl)amino)methyl)pyridin-2-yl)-[1,1′-biphenyl]-3-yl)-4-oxo-4H-pyrido[1,2-a]pyrimidin-3-yl)methyl)amino)-2,2-dimethylpropanoate;
(S)-5-((((3-chloro-5-(2,2′-dichloro-3′-(6-methoxy-5-(((((S)-5-oxopyrrolidin-2-yl)methyl)amino)methyl)pyridin-2-yl)-[1,1′-biphenyl]-3-yl)-1H-pyrrolo[3,2-b]pyridin-2-yl)methyl)amino)methyl)pyrrolidin-2-one;
(R)-5-((((3-chloro-5-(2,2′-dichloro-3′-(6-methoxy-5-(((((S)-5-oxopyrrolidin-2-yl)methyl)amino)methyl)pyridin-2-yl)-[1,1′-biphenyl]-3-yl)-1H-pyrrolo[3,2-b]pyridin-2-yl)methyl)amino)methyl)pyrrolidin-2-one;
(S)-5-((((3-chloro-5-(2,2′-dichloro-3′-(6-methoxy-5-(((((R)-5-oxopyrrolidin-2-yl)methyl)amino)methyl)pyridin-2-yl)-[1,1′-biphenyl]-3-yl)-1H-pyrrolo[3,2-b]pyridin-2-yl)methyl)amino)methyl)pyrrolidin-2-one;
(R)-5-((((3-chloro-5-(2,2′-dichloro-3′-(6-methoxy-5-(((((R)-5-oxopyrrolidin-2-yl)methyl)amino)methyl)pyridin-2-yl)-[1,1′-biphenyl]-3-yl)-1H-pyrrolo[3,2-b]pyridin-2-yl)methyl)amino)methyl)pyrrolidin-2-one;
(S)-5-((((3-chloro-6-(2,2′-dichloro-3′-(6-methoxy-5-(((((S)-5-oxopyrrolidin-2-yl)methyl)amino)methyl)pyridin-2-yl)-[1,1′-biphenyl]-3-yl)-1H-pyrrolo[2,3-b]pyridin-2-yl)methyl)amino)methyl)pyrrolidin-2-one;
(R)-5-((((3-chloro-6-(2,2′-dichloro-3′-(6-methoxy-5-(((((S)-5-oxopyrrolidin-2-yl)methyl)amino)methyl)pyridin-2-yl)-[1,1′-biphenyl]-3-yl)-1H-pyrrolo[2,3-b]pyridin-2-yl)methyl)amino)methyl)pyrrolidin-2-one;
(S)-5-((((3-chloro-6-(2,2′-dichloro-3′-(6-methoxy-5-(((((R)-5-oxopyrrolidin-2-yl)methyl)amino)methyl)pyridin-2-yl)-[1,1′-biphenyl]-3-yl)-1H-pyrrolo[2,3-b]pyridin-2-yl)methyl)amino)methyl)pyrrolidin-2-one;
(R)-5-((((3-chloro-6-(2,2′-dichloro-3′-(6-methoxy-5-(((((R)-5-oxopyrrolidin-2-yl)methyl)amino)methyl)pyridin-2-yl)-[1,1′-biphenyl]-3-yl)-1H-pyrrolo[2,3-b]pyridin-2-yl)methyl)amino)methyl)pyrrolidin-2-one;
2-(2,2′-dichloro-3′-(6-methoxy-5-(((((S)-5-oxopyrrolidin-2-yl)methyl)amino)methyl)pyridin-2-yl)-[1,1′-biphenyl]-3-yl)-5-(((((S)-5-oxopyrrolidin-2-yl)methyl)amino)methyl)isoindolin-1-one;
2-(2,2′-dichloro-3′-(6-methoxy-5-(((((R)-5-oxopyrrolidin-2-yl)methyl)amino)methyl)pyridin-2-yl)-[1,1′-biphenyl]-3-yl)-5-(((((S)-5-oxopyrrolidin-2-yl)methyl)amino)methyl)isoindolin-1-one;
2-(2,2′-dichloro-3′-(6-methoxy-5-(((((S)-5-oxopyrrolidin-2-yl)methyl)amino)methyl)pyridin-2-yl)-[1,1′-biphenyl]-3-yl)-5-(((((R)-5-oxopyrrolidin-2-yl)methyl)amino)methyl)isoindolin-1-one;
2-(2,2′-dichloro-3′-(6-methoxy-5-(((((R)-5-oxopyrrolidin-2-yl)methyl)amino)methyl)pyridin-2-yl)-[1,1′-biphenyl]-3-yl)-5-(((((R)-5-oxopyrrolidin-2-yl)methyl)amino)methyl)isoindolin-1-one;
(S)-5-((((6-(2,2′-dichloro-3′-(1-methyl-3-(((((S)-5-oxopyrrolidin-2-yl)methyl)amino)methyl)-1H-pyrrolo[2,3-b]pyridin-6-yl)-[1,1′-biphenyl]-3-yl)-2-methoxypyridin-3-yl)methyl)amino)methyl)pyrrolidin-2-one;
(R)-5-((((6-(2,2′-dichloro-3′-(1-methyl-3-(((((S)-5-oxopyrrolidin-2-yl)methyl)amino)methyl)-1H-pyrrolo[2,3-b]pyridin-6-yl)-[1,1′-biphenyl]-3-yl)-2-methoxypyridin-3-yl)methyl)amino)methyl)pyrrolidin-2-one;
(S)-5-((((6-(2,2′-dichloro-3′-(1-methyl-3-(((((R)-5-oxopyrrolidin-2-yl)methyl)amino)methyl)-1H-pyrrolo[2,3-b]pyridin-6-yl)-[1,1′-biphenyl]-3-yl)-2-methoxypyridin-3-yl)methyl)amino)methyl)pyrrolidin-2-one;
(R)-5-((((6-(2,2′-dichloro-3′-(1-methyl-3-(((((R)-5-oxopyrrolidin-2-yl)methyl)amino)methyl)-1H-pyrrolo[2,3-b]pyridin-6-yl)-[1,1′-biphenyl]-3-yl)-2-methoxypyridin-3-yl)methyl)amino)methyl)pyrrolidin-2-one;
(S)-5-((((6-(2,2′-dichloro-3′-(3-(((((S)-5-oxopyrrolidin-2-yl)methyl)amino)methyl)imidazo[1,2-a]pyridin-6-yl)-[1,1′-biphenyl]-3-yl)-2-methoxypyridin-3-yl)methyl)amino)methyl)pyrrolidin-2-one;
(R)-5-((((6-(2,2′-dichloro-3′-(3-(((((S)-5-oxopyrrolidin-2-yl)methyl)amino)methyl)imidazo[1,2-a]pyridin-6-yl)-[1,1′-biphenyl]-3-yl)-2-methoxypyridin-3-yl)methyl)amino)methyl)pyrrolidin-2-one;
(S)-5-((((6-(2,2′-dichloro-3′-(3-(((((R)-5-oxopyrrolidin-2-yl)methyl)amino)methyl)imidazo[1,2-a]pyridin-6-yl)-[1,1′-biphenyl]-3-yl)-2-methoxypyridin-3-yl)methyl)amino)methyl)pyrrolidin-2-one;
(R)-5-((((6-(2,2′-dichloro-3′-(3-(((((R)-5-oxopyrrolidin-2-yl)methyl)amino)methyl)imidazo[1,2-a]pyridin-6-yl)-[1,1′-biphenyl]-3-yl)-2-methoxypyridin-3-yl)methyl)amino)methyl)pyrrolidin-2-one;
(5S)-5-((((6-(2,2′-dichloro-3′-((S)-5-(((S)-2-hydroxypropyl)amino)-5,6,7,8-tetrahydroquinolin-2-yl)-[1,1′-biphenyl]-3-yl)-2-methoxypyridin-3-yl)methyl)amino)methyl)pyrrolidin-2-one;
(5R)-5-((((6-(2,2′-dichloro-3′-((S)-5-(((S)-2-hydroxypropyl)amino)-5,6,7,8-tetrahydroquinolin-2-yl)-[1,1′-biphenyl]-3-yl)-2-methoxypyridin-3-yl)methyl)amino)methyl)pyrrolidin-2-one;
(5S)-5-((((6-(2,2′-dichloro-3′-((S)-5-(((R)-2-hydroxypropyl)amino)-5,6,7,8-tetrahydroquinolin-2-yl)-[1,1′-biphenyl]-3-yl)-2-methoxypyridin-3-yl)methyl)amino)methyl)pyrrolidin-2-one;
(5R)-5-((((6-(2,2′-dichloro-3′-((S)-5-(((R)-2-hydroxypropyl)amino)-5,6,7,8-tetrahydroquinolin-2-yl)-[1,1′-biphenyl]-3-yl)-2-methoxypyridin-3-yl)methyl)amino)methyl)pyrrolidin-2-one;
(5S)-5-((((6-(2,2′-dichloro-3′-((R)-5-(((S)-2-hydroxypropyl)amino)-5,6,7,8-tetrahydroquinolin-2-yl)-[1,1′-biphenyl]-3-yl)-2-methoxypyridin-3-yl)methyl)amino)methyl)pyrrolidin-2-one;
(5R)-5-((((6-(2,2′-dichloro-3′-((R)-5-(((S)-2-hydroxypropyl)amino)-5,6,7,8-tetrahydroquinolin-2-yl)-[1,1′-biphenyl]-3-yl)-2-methoxypyridin-3-yl)methyl)amino)methyl)pyrrolidin-2-one;
(5S)-5-((((6-(2,2′-dichloro-3′-((R)-5-(((R)-2-hydroxypropyl)amino)-5,6,7,8-tetrahydroquinolin-2-yl)-[1,1′-biphenyl]-3-yl)-2-methoxypyridin-3-yl)methyl)amino)methyl)pyrrolidin-2-one;
(5R)-5-((((6-(2,2′-dichloro-3′-((R)-5-(((R)-2-hydroxypropyl)amino)-5,6,7,8-tetrahydroquinolin-2-yl)-[1,1′-biphenyl]-3-yl)-2-methoxypyridin-3-yl)methyl)amino)methyl)pyrrolidin-2-one;
(R)-2-(3-(6-(2,2′-dichloro-3′-(6-methoxy-5-((((5-oxopyrrolidin-2-yl)methyl)amino)methyl)pyridin-2-yl)-[1,1′-biphenyl]-3-yl)-3-oxo-[1,2,4]triazolo[4,3-a]pyridin-2(3H)-yl)propyl)isoindoline-1,3-dione;
(S)-2-(3-(6-(2,2′-dichloro-3′-(6-methoxy-5-((((5-oxopyrrolidin-2-yl)methyl)amino)methyl)pyridin-2-yl)-[1,1′-biphenyl]-3-yl)-3-oxo-[1,2,4]triazolo[4,3-a]pyridin-2(3H)-yl)propyl)isoindoline-1,3-dione;
(S)-6-(2,2′-dichloro-3′-(6-methoxy-5-((((5-oxopyrrolidin-2-yl)methyl)amino)methyl)pyridin-2-yl)-[1,1′-biphenyl]-3-yl)-2-(3-(diisobutylamino)propyl)-[1,2,4]triazolo[4,3-a]pyridin-3(2H)-one;
(R)-6-(2,2′-dichloro-3′-(6-methoxy-5-((((5-oxopyrrolidin-2-yl)methyl)amino)methyl)pyridin-2-yl)-[1,1′-biphenyl]-3-yl)-2-(3-(diisobutylamino)propyl)-[1,2,4]triazolo[4,3-a]pyridin-3(2H)-one;
8-(2,2′-dichloro-3′-(4-chloro-6-methoxy-5-(((((S)-5-oxopyrrolidin-2-yl)methyl)amino)methyl)pyridin-2-yl)-[1,1′-biphenyl]-3-yl)-3-((((1r,3r)-3-hydroxy-3-methylcyclobutyl)amino)methyl)-4H-pyrido[1,2-a]pyrimidin-4-one;
8-(2,2′-dichloro-3′-(4-chloro-6-methoxy-5-(((((S)-5-oxopyrrolidin-2-yl)methyl)amino)methyl)pyridin-2-yl)-[1,1′-biphenyl]-3-yl)-3-((((1s,3s)-3-hydroxy-3-methylcyclobutyl)amino)methyl)-4H-pyrido[1,2-a]pyrimidin-4-one;
8-(2,2′-dichloro-3′-(4-chloro-6-methoxy-5-(((((R)-5-oxopyrrolidin-2-yl)methyl)amino)methyl)pyridin-2-yl)-[1,1′-biphenyl]-3-yl)-3-((((1r,3r)-3-hydroxy-3-methylcyclobutyl)amino)methyl)-4H-pyrido[1,2-a]pyrimidin-4-one;
8-(2,2′-dichloro-3′-(4-chloro-6-methoxy-5-(((((R)-5-oxopyrrolidin-2-yl)methyl)amino)methyl)pyridin-2-yl)-[1,1′-biphenyl]-3-yl)-3-((((1s,3s)-3-hydroxy-3-methylcyclobutyl)amino)methyl)-4H-pyrido[1,2-a]pyrimidin-4-one;
(5S,5'S)-5,5′-(((2,2′-dichloro-[1,1′-biphenyl]-3,3′-diyl)bis(8-methoxy-3,4-dihydroisoquinoline-6,2(1H)-diyl))bis(methylene))bis(pyrrolidin-2-one);
(5R,5'S)-5,5′-(((2,2′-dichloro-[1,1′-biphenyl]-3,3′-diyl)bis(8-methoxy-3,4-dihydroisoquinoline-6,2(1H)-diyl))bis(methylene))bis(pyrrolidin-2-one);
(5S,5′R)-5,5′-(((2,2′-dichloro-[1,1′-biphenyl]-3,3′-diyl)bis(8-methoxy-3,4-dihydroisoquinoline-6,2(1H)-diyl))bis(methylene))bis(pyrrolidin-2-one);
(5R,5′R)-5,5′-(((2,2′-dichloro-[1,1′-biphenyl]-3,3′-diyl)bis(8-methoxy-3,4-dihydroisoquinoline-6,2(1H)-diyl))bis(methylene))bis(pyrrolidin-2-one);
(S)-6-(2,2′-dichloro-3′-(6-methoxy-5-((((5-oxopyrrolidin-2-yl)methyl)amino)methyl)pyridin-2-yl)-[1,1′-biphenyl]-3-yl)-2-(((2-hydroxyethyl)(methyl)amino)methyl)-3-methylpyrrolo[2,1-f][1,2,4]triazin-4(3H)-one;
(r)-6-(2,2′-dichloro-3′-(6-methoxy-5-((((5-oxopyrrolidin-2-yl)methyl)amino)methyl)pyridin-2-yl)-[1,1′-biphenyl]-3-yl)-2-(((2-hydroxyethyl)(methyl)amino)methyl)-3-methylpyrrolo[2,1-f][1,2,4]triazin-4(3H)-one;
(S)-10-(2,2′-dichloro-3′-(6-methoxy-5-((((5-oxopyrrolidin-2-yl)methyl)amino)methyl)pyridin-2-yl)-[1,1′-biphenyl]-3-yl)-2,3,4,5-tetrahydro-6H-pyrido[1′,2′:1,2]pyrimido[5,4-f][1,4]oxazepin-6-one;
(R)-10-(2,2′-dichloro-3′-(6-methoxy-5-((((5-oxopyrrolidin-2-yl)methyl)amino)methyl)pyridin-2-yl)-[1,1′-biphenyl]-3-yl)-2,3,4,5-tetrahydro-6H-pyrido[1′,2′:1,2]pyrimido[5,4-f][1,4]oxazepin-6-one;
(S)-5-((((6-(2,2′-dichloro-3′-(4-methoxy-3-(((((S)-5-oxopyrrolidin-2-yl)methyl)amino)methyl)pyrazolo[1,5-a]pyridin-6-yl)-[1,1′-biphenyl]-3-yl)-2-methoxypyridin-3-yl)methyl)amino)methyl)pyrrolidin-2-one;
(R)-5-((((6-(2,2′-dichloro-3′-(4-methoxy-3-(((((S)-5-oxopyrrolidin-2-yl)methyl)amino)methyl)pyrazolo[1,5-a]pyridin-6-yl)-[1,1′-biphenyl]-3-yl)-2-methoxypyridin-3-yl)methyl)amino)methyl)pyrrolidin-2-one;
(S)-5-((((6-(2,2′-dichloro-3′-(4-methoxy-3-(((((R)-5-oxopyrrolidin-2-yl)methyl)amino)methyl)pyrazolo[1,5-a]pyridin-6-yl)-[1,1′-biphenyl]-3-yl)-2-methoxypyridin-3-yl)methyl)amino)methyl)pyrrolidin-2-one;
(R)-5-((((6-(2,2′-dichloro-3′-(4-methoxy-3-(((((R)-5-oxopyrrolidin-2-yl)methyl)amino)methyl)pyrazolo[1,5-a]pyridin-6-yl)-[1,1′-biphenyl]-3-yl)-2-methoxypyridin-3-yl)methyl)amino)methyl)pyrrolidin-2-one;
(S)-5-((((8-chloro-2-(2,2′-dichloro-3′-(4-fluoro-6-methoxy-5-(((((S)-5-oxopyrrolidin-2-yl)methyl)amino)methyl)pyridin-2-yl)-[1,1′-biphenyl]-3-yl)-5-methoxyquinolin-6-yl)methyl)amino)methyl)pyrrolidin-2-one;
(R)-5-((((8-chloro-2-(2,2′-dichloro-3′-(4-fluoro-6-methoxy-5-(((((S)-5-oxopyrrolidin-2-yl)methyl)amino)methyl)pyridin-2-yl)-[1,1′-biphenyl]-3-yl)-5-methoxyquinolin-6-yl)methyl)amino)methyl)pyrrolidin-2-one;
(S)-5-((((8-chloro-2-(2,2′-dichloro-3′-(4-fluoro-6-methoxy-5-(((((R)-5-oxopyrrolidin-2-yl)methyl)amino)methyl)pyridin-2-yl)-[1,1′-biphenyl]-3-yl)-5-methoxyquinolin-6-yl)methyl)amino)methyl)pyrrolidin-2-one;
(R)-5-((((8-chloro-2-(2,2′-dichloro-3′-(4-fluoro-6-methoxy-5-(((((R)-5-oxopyrrolidin-2-yl)methyl)amino)methyl)pyridin-2-yl)-[1,1′-biphenyl]-3-yl)-5-methoxyquinolin-6-yl)methyl)amino)methyl)pyrrolidin-2-one;
8-(2,2′-dichloro-3′-(6-methoxy-5-(((((S)-5-oxopyrrolidin-2-yl)methyl)amino)methyl)pyridin-2-yl)-[1,1′-biphenyl]-3-yl)-2-ethyl-3-(((((S)-5-oxopyrrolidin-2-yl)methyl)amino)methyl)-4H-pyrido[1,2-a]pyrimidin-4-one;
8-(2,2′-dichloro-3′-(6-methoxy-5-(((((R)-5-oxopyrrolidin-2-yl)methyl)amino)methyl)pyridin-2-yl)-[1,1′-biphenyl]-3-yl)-2-ethyl-3-(((((S)-5-oxopyrrolidin-2-yl)methyl)amino)methyl)-4H-pyrido[1,2-a]pyrimidin-4-one;
8-(2,2′-dichloro-3′-(6-methoxy-5-(((((S)-5-oxopyrrolidin-2-yl)methyl)amino)methyl)pyridin-2-yl)-[1,1′-biphenyl]-3-yl)-2-ethyl-3-(((((R)-5-oxopyrrolidin-2-yl)methyl)amino)methyl)-4H-pyrido[1,2-a]pyrimidin-4-one;
8-(2,2′-dichloro-3′-(6-methoxy-5-(((((R)-5-oxopyrrolidin-2-yl)methyl)amino)methyl)pyridin-2-yl)-[1,1′-biphenyl]-3-yl)-2-ethyl-3-(((((R)-5-oxopyrrolidin-2-yl)methyl)amino)methyl)-4H-pyrido[1,2-a]pyrimidin-4-one;
6-(2,2′-dichloro-3′-(6-methoxy-5-(((((S)-5-oxopyrrolidin-2-yl)methyl)amino)methyl)pyridin-2-yl)-[1,1′-biphenyl]-3-yl)-8-fluoro-2-(2-((((S)-5-oxopyrrolidin-2-yl)methyl)amino)ethyl)-3,4-dihydroisoquinolin-1(2H)-one;
6-(2,2′-dichloro-3′-(6-methoxy-5-(((((R)-5-oxopyrrolidin-2-yl)methyl)amino)methyl)pyridin-2-yl)-[1,1′-biphenyl]-3-yl)-8-fluoro-2-(2-((((S)-5-oxopyrrolidin-2-yl)methyl)amino)ethyl)-3,4-dihydroisoquinolin-1(2H)-one;
6-(2,2′-dichloro-3′-(6-methoxy-5-(((((S)-5-oxopyrrolidin-2-yl)methyl)amino)methyl)pyridin-2-yl)-[1,1′-biphenyl]-3-yl)-8-fluoro-2-(2-((((R)-5-oxopyrrolidin-2-yl)methyl)amino)ethyl)-3,4-dihydroisoquinolin-1(2H)-one;
6-(2,2′-dichloro-3′-(6-methoxy-5-(((((R)-5-oxopyrrolidin-2-yl)methyl)amino)methyl)pyridin-2-yl)-[1,1′-biphenyl]-3-yl)-8-fluoro-2-(2-((((R)-5-oxopyrrolidin-2-yl)methyl)amino)ethyl)-3,4-dihydroisoquinolin-1(2H)-one;
(S)-5-((((2-(2,2′-dichloro-3′-(6-methoxy-5-(((((S)-5-oxopyrrolidin-2-yl)methyl)amino)methyl)pyridin-2-yl)-[1,1′-biphenyl]-3-yl)-7-methyl-7H-pyrrolo[2,3-d]pyrimidin-5-yl)methyl)amino)methyl)pyrrolidin-2-one;
(R)-5-((((2-(2,2′-dichloro-3′-(6-methoxy-5-(((((S)-5-oxopyrrolidin-2-yl)methyl)amino)methyl)pyridin-2-yl)-[1,1′-biphenyl]-3-yl)-7-methyl-7H-pyrrolo[2,3-d]pyrimidin-5-yl)methyl)amino)methyl)pyrrolidin-2-one;
(S)-5-((((2-(2,2′-dichloro-3′-(6-methoxy-5-(((((R)-5-oxopyrrolidin-2-yl)methyl)amino)methyl)pyridin-2-yl)-[1,1′-biphenyl]-3-yl)-7-methyl-7H-pyrrolo[2,3-d]pyrimidin-5-yl)methyl)amino)methyl)pyrrolidin-2-one;
(R)-5-((((2-(2,2′-dichloro-3′-(6-methoxy-5-(((((R)-5-oxopyrrolidin-2-yl)methyl)amino)methyl)pyridin-2-yl)-[1,1′-biphenyl]-3-yl)-7-methyl-7H-pyrrolo[2,3-d]pyrimidin-5-yl)methyl)amino)methyl)pyrrolidin-2-one;
(S)-5-((((6-(2,2′-dichloro-3′-(1-methyl-3-(((((S)-5-oxopyrrolidin-2-yl)methyl)amino)methyl)-1H-pyrrolo[3,2-c]pyridin-6-yl)-[1,1′-biphenyl]-3-yl)-2-methoxypyridin-3-yl)methyl)amino)methyl)pyrrolidin-2-one;
(R)-5-((((6-(2,2′-dichloro-3′-(1-methyl-3-(((((S)-5-oxopyrrolidin-2-yl)methyl)amino)methyl)-1H-pyrrolo[3,2-c]pyridin-6-yl)-[1,1′-biphenyl]-3-yl)-2-methoxypyridin-3-yl)methyl)amino)methyl)pyrrolidin-2-one;
(S)-5-((((6-(2,2′-dichloro-3′-(1-methyl-3-(((((R)-5-oxopyrrolidin-2-yl)methyl)amino)methyl)-1H-pyrrolo[3,2-c]pyridin-6-yl)-[1,1′-biphenyl]-3-yl)-2-methoxypyridin-3-yl)methyl)amino)methyl)pyrrolidin-2-one;
(R)-5-((((6-(2,2′-dichloro-3′-(1-methyl-3-(((((R)-5-oxopyrrolidin-2-yl)methyl)amino)methyl)-1H-pyrrolo[3,2-c]pyridin-6-yl)-[1,1′-biphenyl]-3-yl)-2-methoxypyridin-3-yl)methyl)amino)methyl)pyrrolidin-2-one;
(S)-7-(2,2′-dichloro-3′-(6-methoxy-5-((((5-oxopyrrolidin-2-yl)methyl)amino)methyl)pyridin-2-yl)-[1,1′-biphenyl]-3-yl)-1,3,4,5-tetrahydro-2H-pyrido[2,3-e][1,4]diazepin-2-one;
(R)-7-(2,2′-dichloro-3′-(6-methoxy-5-((((5-oxopyrrolidin-2-yl)methyl)amino)methyl)pyridin-2-yl)-[1,1′-biphenyl]-3-yl)-1,3,4,5-tetrahydro-2H-pyrido[2,3-e][1,4]diazepin-2-one;
8-(2,2′-dichloro-3′-(4-fluoro-6-methoxy-5-(((((S)-5-oxopyrrolidin-2-yl)methyl)amino)methyl)pyridin-2-yl)-[1,1′-biphenyl]-3-yl)-2-methyl-3-(((((S)-5-oxopyrrolidin-2-yl)methyl)amino)methyl)-4H-pyrido[1,2-a]pyrimidin-4-one;
8-(2,2′-dichloro-3′-(4-fluoro-6-methoxy-5-(((((R)-5-oxopyrrolidin-2-yl)methyl)amino)methyl)pyridin-2-yl)-[1,1′-biphenyl]-3-yl)-2-methyl-3-(((((S)-5-oxopyrrolidin-2-yl)methyl)amino)methyl)-4H-pyrido[1,2-a]pyrimidin-4-one;
8-(2,2′-dichloro-3′-(4-fluoro-6-methoxy-5-(((((S)-5-oxopyrrolidin-2-yl)methyl)amino)methyl)pyridin-2-yl)-[1,1′-biphenyl]-3-yl)-2-methyl-3-(((((R)-5-oxopyrrolidin-2-yl)methyl)amino)methyl)-4H-pyrido[1,2-a]pyrimidin-4-one;
8-(2,2′-dichloro-3′-(4-fluoro-6-methoxy-5-(((((R)-5-oxopyrrolidin-2-yl)methyl)amino)methyl)pyridin-2-yl)-[1,1′-biphenyl]-3-yl)-2-methyl-3-(((((R)-5-oxopyrrolidin-2-yl)methyl)amino)methyl)-4H-pyrido[1,2-a]pyrimidin-4-one;
(S)-6-(2,2′-dichloro-3′-(6-methoxy-5-((((5-oxopyrrolidin-2-yl)methyl)amino)methyl)pyridin-2-yl)-[1,1′-biphenyl]-3-yl)-2-(((2-hydroxy-2-methylpropyl)amino)methyl)-3-methylpyrrolo[2,1-f][1,2,4]triazin-4(3H)-one;
(R)-6-(2,2′-dichloro-3′-(6-methoxy-5-((((5-oxopyrrolidin-2-yl)methyl)amino)methyl)pyridin-2-yl)-[1,1′-biphenyl]-3-yl)-2-(((2-hydroxy-2-methylpropyl)amino)methyl)-3-methylpyrrolo[2,1-f][1,2,4]triazin-4(3H)-one;
(S)-6-(2,2′-dichloro-3′-(6-methoxy-5-((((5-oxopyrrolidin-2-yl)methyl)amino)methyl)pyridin-2-yl)-[1,1′-biphenyl]-3-yl)-3-methyl-2-(((3,3,3-trifluoropropyl)amino)methyl)pyrrolo[2,1-f][1,2,4]triazin-4(3H)-one;
(R)-6-(2,2′-dichloro-3′-(6-methoxy-5-((((5-oxopyrrolidin-2-yl)methyl)amino)methyl)pyridin-2-yl)-[1,1′-biphenyl]-3-yl)-3-methyl-2-(((3,3,3-trifluoropropyl)amino)methyl)pyrrolo[2,1-f][1,2,4]triazin-4(3H)-one;
(S)-8-(2,2′-dichloro-3′-(1-methyl-2,3,4,5-tetrahydro-1H-benzo[e][1,4]diazepin-8-yl)-[1,1′-biphenyl]-3-yl)-3-((((5-oxopyrrolidin-2-yl)methyl)amino)methyl)-4H-pyrido[1,2-a]pyrimidin-4-one;
(R)-8-(2,2′-dichloro-3′-(1-methyl-2,3,4,5-tetrahydro-1H-benzo[e][1,4]diazepin-8-yl)-[1,1′-biphenyl]-3-yl)-3-((((5-oxopyrrolidin-2-yl)methyl)amino)methyl)-4H-pyrido[1,2-a]pyrimidin-4-one;
(S)-5-((((6-(2,2′-dichloro-3′-(3-((((S)-2-hydroxypropyl)amino)methyl)-1-methyl-1H-pyrrolo[2,3-b]pyridin-6-yl)-[1,1′-biphenyl]-3-yl)-2-methoxypyridin-3-yl)methyl)amino)methyl)pyrrolidin-2-one;
(R)-5-((((6-(2,2′-dichloro-3′-(3-((((S)-2-hydroxypropyl)amino)methyl)-1-methyl-1H-pyrrolo[2,3-b]pyridin-6-yl)-[1,1′-biphenyl]-3-yl)-2-methoxypyridin-3-yl)methyl)amino)methyl)pyrrolidin-2-one;
(S)-5-((((6-(2,2′-dichloro-3′-(3-((((R)-2-hydroxypropyl)amino)methyl)-1-methyl-1H-pyrrolo[2,3-b]pyridin-6-yl)-[1,1′-biphenyl]-3-yl)-2-methoxypyridin-3-yl)methyl)amino)methyl)pyrrolidin-2-one;
(R)-5-((((6-(2,2′-dichloro-3′-(3-((((R)-2-hydroxypropyl)amino)methyl)-1-methyl-1H-pyrrolo[2,3-b]pyridin-6-yl)-[1,1′-biphenyl]-3-yl)-2-methoxypyridin-3-yl)methyl)amino)methyl)pyrrolidin-2-one;
(S)-5-((((6-(2,2′-dichloro-3′-(3-(((2-hydroxyethyl)(methyl)amino)methyl)-1-methyl-1H-pyrrolo[2,3-b]pyridin-6-yl)-[1,1′-biphenyl]-3-yl)-2-methoxypyridin-3-yl)methyl)amino)methyl)pyrrolidin-2-one;
(R)-5-((((6-(2,2′-dichloro-3′-(3-(((2-hydroxyethyl)(methyl)amino)methyl)-1-methyl-1H-pyrrolo[2,3-b]pyridin-6-yl)-[1,1′-biphenyl]-3-yl)-2-methoxypyridin-3-yl)methyl)amino)methyl)pyrrolidin-2-one;
(S)-5-((((6-(2,2′-dichloro-3′-(1-methyl-3-(((((S)-oxetan-2-yl)methyl)amino)methyl)-1H-pyrrolo[2,3-b]pyridin-6-yl)-[1,1′-biphenyl]-3-yl)-2-methoxypyridin-3-yl)methyl)amino)methyl)pyrrolidin-2-one;
(R)-5-((((6-(2,2′-dichloro-3′-(1-methyl-3-(((((S)-oxetan-2-yl)methyl)amino)methyl)-1H-pyrrolo[2,3-b]pyridin-6-yl)-[1,1′-biphenyl]-3-yl)-2-methoxypyridin-3-yl)methyl)amino)methyl)pyrrolidin-2-one;
(S)-5-((((6-(2,2′-dichloro-3′-(1-methyl-3-(((((R)-oxetan-2-yl)methyl)amino)methyl)-1H-pyrrolo[2,3-b]pyridin-6-yl)-[1,1′-biphenyl]-3-yl)-2-methoxypyridin-3-yl)methyl)amino)methyl)pyrrolidin-2-one;
(R)-5-((((6-(2,2′-dichloro-3′-(1-methyl-3-(((((R)-oxetan-2-yl)methyl)amino)methyl)-1H-pyrrolo[2,3-b]pyridin-6-yl)-[1,1′-biphenyl]-3-yl)-2-methoxypyridin-3-yl)methyl)amino)methyl)pyrrolidin-2-one;
(S)-5-((((6-(2,2′-dichloro-3′-(1-methyl-3-(((2-(2-oxopyrrolidin-1-yl)ethyl)amino)methyl)-1H-pyrrolo[2,3-b]pyridin-6-yl)-[1,1′-biphenyl]-3-yl)-2-methoxypyridin-3-yl)methyl)amino)methyl)pyrrolidin-2-one;
(R)-5-((((6-(2,2′-dichloro-3′-(1-methyl-3-(((2-(2-oxopyrrolidin-1-yl)ethyl)amino)methyl)-1H-pyrrolo[2,3-b]pyridin-6-yl)-[1,1′-biphenyl]-3-yl)-2-methoxypyridin-3-yl)methyl)amino)methyl)pyrrolidin-2-one;
(S)-5-((((6-(3′-(3-(((1-acetylpiperidin-4-yl)amino)methyl)-1-methyl-1H-pyrrolo[2,3-b]pyridin-6-yl)-2,2′-dichloro-[1,1′-biphenyl]-3-yl)-2-methoxypyridin-3-yl)methyl)amino)methyl)pyrrolidin-2-one;
(R)-5-((((6-(3′-(3-(((1-acetylpiperidin-4-yl)amino)methyl)-1-methyl-1H-pyrrolo[2,3-b]pyridin-6-yl)-2,2′-dichloro-[1,1′-biphenyl]-3-yl)-2-methoxypyridin-3-yl)methyl)amino)methyl)pyrrolidin-2-one;
8-(2,2′-dichloro-3′-(1-methyl-3-(((((S)-5-oxopyrrolidin-2-yl)methyl)amino)methyl)-1H-pyrrolo[2,3-b]pyridin-6-yl)-[1,1′-biphenyl]-3-yl)-3-(((((S)-5-oxopyrrolidin-2-yl)methyl)amino)methyl)-4H-pyrido[1,2-a]pyrimidin-4-one;
8-(2,2′-dichloro-3′-(1-methyl-3-(((((R)-5-oxopyrrolidin-2-yl)methyl)amino)methyl)-1H-pyrrolo[2,3-b]pyridin-6-yl)-[1,1′-biphenyl]-3-yl)-3-(((((S)-5-oxopyrrolidin-2-yl)methyl)amino)methyl)-4H-pyrido[1,2-a]pyrimidin-4-one;
8-(2,2′-dichloro-3′-(1-methyl-3-(((((S)-5-oxopyrrolidin-2-yl)methyl)amino)methyl)-1H-pyrrolo[2,3-b]pyridin-6-yl)-[1,1′-biphenyl]-3-yl)-3-(((((R)-5-oxopyrrolidin-2-yl)methyl)amino)methyl)-4H-pyrido[1,2-a]pyrimidin-4-one;
8-(2,2′-dichloro-3′-(1-methyl-3-(((((R)-5-oxopyrrolidin-2-yl)methyl)amino)methyl)-1H-pyrrolo[2,3-b]pyridin-6-yl)-[1,1′-biphenyl]-3-yl)-3-(((((R)-5-oxopyrrolidin-2-yl)methyl)amino)methyl)-4H-pyrido[1,2-a]pyrimidin-4-one;
(5S,5'S)-5,5′-(((((2,2′-dichloro-[1,1′-biphenyl]-3,3′-diyl)bis(1-methyl-1H-pyrrolo[2,3-b]pyridine-6,3-diyl))bis(methylene))bis(azanediyl))bis(methylene))bis(pyrrolidin-2-one);
(5R,5'S)-5,5′-(((((2,2′-dichloro-[1,1′-biphenyl]-3,3′-diyl)bis(1-methyl-1H-pyrrolo[2,3-b]pyridine-6,3-diyl))bis(methylene))bis(azanediyl))bis(methylene))bis(pyrrolidin-2-one);
(5S,5′R)-5,5′-(((((2,2′-dichloro-[1,1′-biphenyl]-3,3′-diyl)bis(1-methyl-1H-pyrrolo[2,3-b]pyridine-6,3-diyl))bis(methylene))bis(azanediyl))bis(methylene))bis(pyrrolidin-2-one);
(5R,5′R)-5,5′-(((((2,2′-dichloro-[1,1′-biphenyl]-3,3′-diyl)bis(1-methyl-1H-pyrrolo[2,3-b]pyridine-6,3-diyl))bis(methylene))bis(azanediyl))bis(methylene))bis(pyrrolidin-2-one);
(S)-5-((((6-(2,2′-dichloro-3′-(2-((S)-2-hydroxypropyl)-8-methoxy-1,2,3,4-tetrahydroisoquinolin-6-yl)-[1,1′-biphenyl]-3-yl)-2-methoxypyridin-3-yl)methyl)amino)methyl)pyrrolidin-2-one;
(R)-5-((((6-(2,2′-dichloro-3′-(2-((S)-2-hydroxypropyl)-8-methoxy-1,2,3,4-tetrahydroisoquinolin-6-yl)-[1,1′-biphenyl]-3-yl)-2-methoxypyridin-3-yl)methyl)amino)methyl)pyrrolidin-2-one;
(S)-5-((((6-(2,2′-dichloro-3′-(2-((R)-2-hydroxypropyl)-8-methoxy-1,2,3,4-tetrahydroisoquinolin-6-yl)-[1,1′-biphenyl]-3-yl)-2-methoxypyridin-3-yl)methyl)amino)methyl)pyrrolidin-2-one;
(R)-5-((((6-(2,2′-dichloro-3′-(2-((R)-2-hydroxypropyl)-8-methoxy-1,2,3,4-tetrahydroisoquinolin-6-yl)-[1,1′-biphenyl]-3-yl)-2-methoxypyridin-3-yl)methyl)amino)methyl)pyrrolidin-2-one;
(S)-5-((((6-(2,2′-dichloro-3′-(2-((R)-2-hydroxypropyl)-8-methoxy-1,2,3,4-tetrahydroisoquinolin-6-yl)-[1,1′-biphenyl]-3-yl)-2-methoxypyridin-3-yl)methyl)amino)methyl)pyrrolidin-2-one;
(R)-5-((((6-(2,2′-dichloro-3′-(2-((S)-2-hydroxypropyl)-8-methoxy-1,2,3,4-tetrahydroisoquinolin-6-yl)-[1,1′-biphenyl]-3-yl)-2-methoxypyridin-3-yl)methyl)amino)methyl)pyrrolidin-2-one;
(S)-5-((((6-(2,2′-dichloro-3′-(2-((S)-2-hydroxypropyl)-8-methoxy-1,2,3,4-tetrahydroisoquinolin-6-yl)-[1,1′-biphenyl]-3-yl)-2-methoxypyridin-3-yl)methyl)amino)methyl)pyrrolidin-2-one;
(R)-5-((((6-(2,2′-dichloro-3′-(2-((R)-2-hydroxypropyl)-8-methoxy-1,2,3,4-tetrahydroisoquinolin-6-yl)-[1,1′-biphenyl]-3-yl)-2-methoxypyridin-3-yl)methyl)amino)methyl)pyrrolidin-2-one;
(S)-5-((((6-(2,2′-dichloro-3′-(2-methyl-3-(((((S)-5-oxopyrrolidin-2-yl)methyl)amino)methyl)pyrazolo[1,5-a]pyrimidin-6-yl)-[1,1′-biphenyl]-3-yl)-2-methoxypyridin-3-yl)methyl)amino)methyl)pyrrolidin-2-one;
(R)-5-((((6-(2,2′-dichloro-3′-(2-methyl-3-(((((S)-5-oxopyrrolidin-2-yl)methyl)amino)methyl)pyrazolo[1,5-a]pyrimidin-6-yl)-[1,1′-biphenyl]-3-yl)-2-methoxypyridin-3-yl)methyl)amino)methyl)pyrrolidin-2-one;
(S)-5-((((6-(2,2′-dichloro-3′-(2-methyl-3-(((((S)-5-oxopyrrolidin-2-yl)methyl)amino)methyl)pyrazolo[1,5-a]pyrimidin-6-yl)-[1,1′-biphenyl]-3-yl)-2-methoxypyridin-3-yl)methyl)amino)methyl)pyrrolidin-2-one;
(R)-5-((((6-(2,2′-dichloro-3′-(2-methyl-3-(((((R)-5-oxopyrrolidin-2-yl)methyl)amino)methyl)pyrazolo[1,5-a]pyrimidin-6-yl)-[1,1′-biphenyl]-3-yl)-2-methoxypyridin-3-yl)methyl)amino)methyl)pyrrolidin-2-one;
7-(2,2′-dichloro-3′-(6-methoxy-5-(((((S)-5-oxopyrrolidin-2-yl)methyl)amino)methyl)pyridin-2-yl)-[1,1′-biphenyl]-3-yl)-2-(((((S)-5-oxopyrrolidin-2-yl)methyl)amino)methyl)quinazolin-4(1H)-one;
7-(2,2′-dichloro-3′-(6-methoxy-5-(((((R)-5-oxopyrrolidin-2-yl)methyl)amino)methyl)pyridin-2-yl)-[1,1′-biphenyl]-3-yl)-2-(((((S)-5-oxopyrrolidin-2-yl)methyl)amino)methyl)quinazolin-4(1H)-one;
7-(2,2′-dichloro-3′-(6-methoxy-5-(((((S)-5-oxopyrrolidin-2-yl)methyl)amino)methyl)pyridin-2-yl)-[1,1′-biphenyl]-3-yl)-2-(((((R)-5-oxopyrrolidin-2-yl)methyl)amino)methyl)quinazolin-4(1H)-one;
7-(2,2′-dichloro-3′-(6-methoxy-5-(((((R)-5-oxopyrrolidin-2-yl)methyl)amino)methyl)pyridin-2-yl)-[1,1′-biphenyl]-3-yl)-2-(((((R)-5-oxopyrrolidin-2-yl)methyl)amino)methyl)quinazolin-4(1H)-one;
(S)-5-((((6-(2,2′-dichloro-3′-(2-(((((S)-5-oxopyrrolidin-2-yl)methyl)amino)methyl)quinazolin-6-yl)-[1,1′-biphenyl]-3-yl)-2-methoxypyridin-3-yl)methyl)amino)methyl)pyrrolidin-2-one;
(R)-5-((((6-(2,2′-dichloro-3′-(2-(((((S)-5-oxopyrrolidin-2-yl)methyl)amino)methyl)quinazolin-6-yl)-[1,1′-biphenyl]-3-yl)-2-methoxypyridin-3-yl)methyl)amino)methyl)pyrrolidin-2-one;
(S)-5-((((6-(2,2′-dichloro-3′-(2-(((((R)-5-oxopyrrolidin-2-yl)methyl)amino)methyl)quinazolin-6-yl)-[1,1′-biphenyl]-3-yl)-2-methoxypyridin-3-yl)methyl)amino)methyl)pyrrolidin-2-one;
(R)-5-((((6-(2,2′-dichloro-3′-(2-(((((R)-5-oxopyrrolidin-2-yl)methyl)amino)methyl)quinazolin-6-yl)-[1,1′-biphenyl]-3-yl)-2-methoxypyridin-3-yl)methyl)amino)methyl)pyrrolidin-2-one;
tert-butyl (S)-((6-(2,2′-dichloro-3′-(2-(hydroxymethyl)-3-methyl-4-oxo-3,4-dihydropyrrolo[2,1-f][1,2,4]triazin-6-yl)-[1,1′-biphenyl]-3-yl)-2-methoxypyridin-3-yl)methyl)((5-oxopyrrolidin-2-yl)methyl)carbamate;
tert-butyl (r)-((6-(2,2′-dichloro-3′-(2-(hydroxymethyl)-3-methyl-4-oxo-3,4-dihydropyrrolo[2,1-f][1,2,4]triazin-6-yl)-[1,1′-biphenyl]-3-yl)-2-methoxypyridin-3-yl)methyl)((5-oxopyrrolidin-2-yl)methyl)carbamate;
(S)-5-((((6-(2,2′-dichloro-3′-(5-methoxy-2-(((S)-5-oxopyrrolidin-2-yl)methyl)-1,2,3,4-tetrahydroisoquinolin-7-yl)-[1,1′-biphenyl]-3-yl)-2-methoxypyridin-3-yl)methyl)amino)methyl)pyrrolidin-2-one;
(R)-5-((((6-(2,2′-dichloro-3′-(5-methoxy-2-(((S)-5-oxopyrrolidin-2-yl)methyl)-1,2,3,4-tetrahydroisoquinolin-7-yl)-[1,1′-biphenyl]-3-yl)-2-methoxypyridin-3-yl)methyl)amino)methyl)pyrrolidin-2-one;
(S)-5-((((6-(2,2′-dichloro-3′-(5-methoxy-2-(((R)-5-oxopyrrolidin-2-yl)methyl)-1,2,3,4-tetrahydroisoquinolin-7-yl)-[1,1′-biphenyl]-3-yl)-2-methoxypyridin-3-yl)methyl)amino)methyl)pyrrolidin-2-one;
(R)-5-((((6-(2,2′-dichloro-3′-(5-methoxy-2-(((R)-5-oxopyrrolidin-2-yl)methyl)-1,2,3,4-tetrahydroisoquinolin-7-yl)-[1,1′-biphenyl]-3-yl)-2-methoxypyridin-3-yl)methyl)amino)methyl)pyrrolidin-2-one;
(S)-5-((6-(2,2′-dichloro-3′-(6-methoxy-5-(((((S)-5-oxopyrrolidin-2-yl)methyl)amino)methyl)pyridin-2-yl)-[1,1′-biphenyl]-3-yl)-8-fluoro-3,4-dihydroisoquinolin-2(1H)-yl)methyl)pyrrolidin-2-one;
(R)-5-((6-(2,2′-dichloro-3′-(6-methoxy-5-(((((S)-5-oxopyrrolidin-2-yl)methyl)amino)methyl)pyridin-2-yl)-[1,1′-biphenyl]-3-yl)-8-fluoro-3,4-dihydroisoquinolin-2(1H)-yl)methyl)pyrrolidin-2-one;
(S)-5-((6-(2,2′-dichloro-3′-(6-methoxy-5-(((((R)-5-oxopyrrolidin-2-yl)methyl)amino)methyl)pyridin-2-yl)-[1,1′-biphenyl]-3-yl)-8-fluoro-3,4-dihydroisoquinolin-2(1H)-yl)methyl)pyrrolidin-2-one;
(R)-5-((6-(2,2′-dichloro-3′-(6-methoxy-5-(((((R)-5-oxopyrrolidin-2-yl)methyl)amino)methyl)pyridin-2-yl)-[1,1′-biphenyl]-3-yl)-8-fluoro-3,4-dihydroisoquinolin-2(1H)-yl)methyl)pyrrolidin-2-one;
(S)-5-((((6-(2,2′-dichloro-3′-(6-(((S)-5-oxopyrrolidin-2-yl)methyl)-5,6,7,8-tetrahydro-1,6-naphthyridin-2-yl)-[1,1′-biphenyl]-3-yl)-2-methoxypyridin-3-yl)methyl)amino)methyl)pyrrolidin-2-one;
(R)-5-((((6-(2,2′-dichloro-3′-(6-(((S)-5-oxopyrrolidin-2-yl)methyl)-5,6,7,8-tetrahydro-1,6-naphthyridin-2-yl)-[1,1′-biphenyl]-3-yl)-2-methoxypyridin-3-yl)methyl)amino)methyl)pyrrolidin-2-one;
(S)-5-((((6-(2,2′-dichloro-3′-(6-(((R)-5-oxopyrrolidin-2-yl)methyl)-5,6,7,8-tetrahydro-1,6-naphthyridin-2-yl)-[1,1′-biphenyl]-3-yl)-2-methoxypyridin-3-yl)methyl)amino)methyl)pyrrolidin-2-one;
(R)-5-((((6-(2,2′-dichloro-3′-(6-(((R)-5-oxopyrrolidin-2-yl)methyl)-5,6,7,8-tetrahydro-1,6-naphthyridin-2-yl)-[1,1′-biphenyl]-3-yl)-2-methoxypyridin-3-yl)methyl)amino)methyl)pyrrolidin-2-one;
(S)-5-((8-chloro-7-(2,2′-dichloro-3′-(6-methoxy-5-(((((S)-5-oxopyrrolidin-2-yl)methyl)amino)methyl)pyridin-2-yl)-[1,1′-biphenyl]-3-yl)-3,4-dihydroisoquinolin-2(1H)-yl)methyl)pyrrolidin-2-one;
(R)-5-((8-chloro-7-(2,2′-dichloro-3′-(6-methoxy-5-(((((S)-5-oxopyrrolidin-2-yl)methyl)amino)methyl)pyridin-2-yl)-[1,1′-biphenyl]-3-yl)-3,4-dihydroisoquinolin-2(1H)-yl)methyl)pyrrolidin-2-one;
(S)-5-((8-chloro-7-(2,2′-dichloro-3′-(6-methoxy-5-(((((R)-5-oxopyrrolidin-2-yl)methyl)amino)methyl)pyridin-2-yl)-[1,1′-biphenyl]-3-yl)-3,4-dihydroisoquinolin-2(1H)-yl)methyl)pyrrolidin-2-one;
(R)-5-((8-chloro-7-(2,2′-dichloro-3′-(6-methoxy-5-(((((R)-5-oxopyrrolidin-2-yl)methyl)amino)methyl)pyridin-2-yl)-[1,1′-biphenyl]-3-yl)-3,4-dihydroisoquinolin-2(1H)-yl)methyl)pyrrolidin-2-one;
(S)-5-((7-(2,2′-dichloro-3′-(6-methoxy-5-(((((S)-5-oxopyrrolidin-2-yl)methyl)amino)methyl)pyridin-2-yl)-[1,1′-biphenyl]-3-yl)-8-fluoro-3,4-dihydroisoquinolin-2(1H)-yl)methyl)pyrrolidin-2-one;
(R)-5-((7-(2,2′-dichloro-3′-(6-methoxy-5-(((((S)-5-oxopyrrolidin-2-yl)methyl)amino)methyl)pyridin-2-yl)-[1,1′-biphenyl]-3-yl)-8-fluoro-3,4-dihydroisoquinolin-2(1H)-yl)methyl)pyrrolidin-2-one;
(S)-5-((7-(2,2′-dichloro-3′-(6-methoxy-5-(((((R)-5-oxopyrrolidin-2-yl)methyl)amino)methyl)pyridin-2-yl)-[1,1′-biphenyl]-3-yl)-8-fluoro-3,4-dihydroisoquinolin-2(1H)-yl)methyl)pyrrolidin-2-one;
(R)-5-((7-(2,2′-dichloro-3′-(6-methoxy-5-(((((R)-5-oxopyrrolidin-2-yl)methyl)amino)methyl)pyridin-2-yl)-[1,1′-biphenyl]-3-yl)-8-fluoro-3,4-dihydroisoquinolin-2(1H)-yl)methyl)pyrrolidin-2-one;
8-(2,2′-dichloro-3′-(3-chloro-2-(((((S)-5-oxopyrrolidin-2-yl)methyl)amino)methyl)imidazo[1,2-a]pyridin-7-yl)-[1,1′-biphenyl]-3-yl)-3-(((((S)-5-oxopyrrolidin-2-yl)methyl)amino)methyl)-4H-pyrido[1,2-a]pyrimidin-4-one;
8-(2,2′-dichloro-3′-(3-chloro-2-(((((R)-5-oxopyrrolidin-2-yl)methyl)amino)methyl)imidazo[1,2-a]pyridin-7-yl)-[1,1′-biphenyl]-3-yl)-3-(((((S)-5-oxopyrrolidin-2-yl)methyl)amino)methyl)-4H-pyrido[1,2-a]pyrimidin-4-one;
8-(2,2′-dichloro-3′-(3-chloro-2-(((((S)-5-oxopyrrolidin-2-yl)methyl)amino)methyl)imidazo[1,2-a]pyridin-7-yl)-[1,1′-biphenyl]-3-yl)-3-(((((R)-5-oxopyrrolidin-2-yl)methyl)amino)methyl)-4H-pyrido[1,2-a]pyrimidin-4-one;
8-(2,2′-dichloro-3′-(3-chloro-2-(((((R)-5-oxopyrrolidin-2-yl)methyl)amino)methyl)imidazo[1,2-a]pyridin-7-yl)-[1,1′-biphenyl]-3-yl)-3-(((((R)-5-oxopyrrolidin-2-yl)methyl)amino)methyl)-4H-pyrido[1,2-a]pyrimidin-4-one;
(5S,5'S)-5,5′-(((((2,2′-dichloro-[1,1′-biphenyl]-3,3′-diyl)bis(3-chloroimidazo[1,2-a]pyridine-7,2-diyl))bis(methylene))bis(azanediyl))bis(methylene))bis(pyrrolidin-2-one);
(5R,5'S)-5,5′-(((((2,2′-dichloro-[1,1′-biphenyl]-3,3′-diyl)bis(3-chloroimidazo[1,2-a]pyridine-7,2-diyl))bis(methylene))bis(azanediyl))bis(methylene))bis(pyrrolidin-2-one);
(5S,5′R)-5,5′-(((((2,2′-dichloro-[1,1′-biphenyl]-3,3′-diyl)bis(3-chloroimidazo[1,2-a]pyridine-7,2-diyl))bis(methylene))bis(azanediyl))bis(methylene))bis(pyrrolidin-2-one);
(5R,5′R)-5,5′-(((((2,2′-dichloro-[1,1′-biphenyl]-3,3′-diyl)bis(3-chloroimidazo[1,2-a]pyridine-7,2-diyl))bis(methylene))bis(azanediyl))bis(methylene))bis(pyrrolidin-2-one);
8-(2,2′-dichloro-3′-(6-chloro-4-methoxy-5-(((((S)-5-oxopyrrolidin-2-yl)methyl)amino)methyl)pyridin-2-yl)-[1,1′-biphenyl]-3-yl)-3-(((((S)-5-oxopyrrolidin-2-yl)methyl)amino)methyl)-4H-pyrido[1,2-a]pyrimidin-4-one;
8-(2,2′-dichloro-3′-(6-chloro-4-methoxy-5-(((((R)-5-oxopyrrolidin-2-yl)methyl)amino)methyl)pyridin-2-yl)-[1,1′-biphenyl]-3-yl)-3-(((((S)-5-oxopyrrolidin-2-yl)methyl)amino)methyl)-4H-pyrido[1,2-a]pyrimidin-4-one;
8-(2,2′-dichloro-3′-(6-chloro-4-methoxy-5-(((((S)-5-oxopyrrolidin-2-yl)methyl)amino)methyl)pyridin-2-yl)-[1,1′-biphenyl]-3-yl)-3-(((((R)-5-oxopyrrolidin-2-yl)methyl)amino)methyl)-4H-pyrido[1,2-a]pyrimidin-4-one;
8-(2,2′-dichloro-3′-(6-chloro-4-methoxy-5-(((((R)-5-oxopyrrolidin-2-yl)methyl)amino)methyl)pyridin-2-yl)-[1,1′-biphenyl]-3-yl)-3-(((((R)-5-oxopyrrolidin-2-yl)methyl)amino)methyl)-4H-pyrido[1,2-a]pyrimidin-4-one;
(S)-5-((((6-(2,2′-dichloro-3′-((S)-8-((((S)-5-oxopyrrolidin-2-yl)methyl)amino)-5,6,7,8-tetrahydroquinolin-3-yl)-[1,1′-biphenyl]-3-yl)-2-methoxypyridin-3-yl)methyl)amino)methyl)pyrrolidin-2-one;
(S)-5-((((6-(2,2′-dichloro-3′-((R)-8-((((S)-5-oxopyrrolidin-2-yl)methyl)amino)-5,6,7,8-tetrahydroquinolin-3-yl)-[1,1′-biphenyl]-3-yl)-2-methoxypyridin-3-yl)methyl)amino)methyl)pyrrolidin-2-one;
(S)-5-((((6-(2,2′-dichloro-3′-((S)-8-((((R)-5-oxopyrrolidin-2-yl)methyl)amino)-5,6,7,8-tetrahydroquinolin-3-yl)-[1,1′-biphenyl]-3-yl)-2-methoxypyridin-3-yl)methyl)amino)methyl)pyrrolidin-2-one;
(S)-5-((((6-(2,2′-dichloro-3′-((R)-8-((((R)-5-oxopyrrolidin-2-yl)methyl)amino)-5,6,7,8-tetrahydroquinolin-3-yl)-[1,1′-biphenyl]-3-yl)-2-methoxypyridin-3-yl)methyl)amino)methyl)pyrrolidin-2-one;
(R)-5-((((6-(2,2′-dichloro-3′-((S)-8-((((S)-5-oxopyrrolidin-2-yl)methyl)amino)-5,6,7,8-tetrahydroquinolin-3-yl)-[1,1′-biphenyl]-3-yl)-2-methoxypyridin-3-yl)methyl)amino)methyl)pyrrolidin-2-one;
(R)-5-((((6-(2,2′-dichloro-3′-((R)-8-((((S)-5-oxopyrrolidin-2-yl)methyl)amino)-5,6,7,8-tetrahydroquinolin-3-yl)-[1,1′-biphenyl]-3-yl)-2-methoxypyridin-3-yl)methyl)amino)methyl)pyrrolidin-2-one;
(R)-5-((((6-(2,2′-dichloro-3′-((S)-8-((((R)-5-oxopyrrolidin-2-yl)methyl)amino)-5,6,7,8-tetrahydroquinolin-3-yl)-[1,1′-biphenyl]-3-yl)-2-methoxypyridin-3-yl)methyl)amino)methyl)pyrrolidin-2-one;
(R)-5-((((6-(2,2′-dichloro-3′-((R)-8-((((R)-5-oxopyrrolidin-2-yl)methyl)amino)-5,6,7,8-tetrahydroquinolin-3-yl)-[1,1′-biphenyl]-3-yl)-2-methoxypyridin-3-yl)methyl)amino)methyl)pyrrolidin-2-one;
6-(2,2′-dichloro-3′-(6-methoxy-5-(((((S)-5-oxopyrrolidin-2-yl)methyl)amino)methyl)pyridin-2-yl)-[1,1′-biphenyl]-3-yl)-2-(((((S)-5-oxopyrrolidin-2-yl)methyl)amino)methyl)quinazolin-4(1H)-one;
6-(2,2′-dichloro-3′-(6-methoxy-5-(((((R)-5-oxopyrrolidin-2-yl)methyl)amino)methyl)pyridin-2-yl)-[1,1′-biphenyl]-3-yl)-2-(((((S)-5-oxopyrrolidin-2-yl)methyl)amino)methyl)quinazolin-4(1H)-one;
6-(2,2′-dichloro-3′-(6-methoxy-5-(((((S)-5-oxopyrrolidin-2-yl)methyl)amino)methyl)pyridin-2-yl)-[1,1′-biphenyl]-3-yl)-2-(((((R)-5-oxopyrrolidin-2-yl)methyl)amino)methyl)quinazolin-4(1H)-one;
6-(2,2′-dichloro-3′-(6-methoxy-5-(((((R)-5-oxopyrrolidin-2-yl)methyl)amino)methyl)pyridin-2-yl)-[1,1′-biphenyl]-3-yl)-2-(((((R)-5-oxopyrrolidin-2-yl)methyl)amino)methyl)quinazolin-4(1H)-one;
(S)-5-((((6-(2,2′-dichloro-3′-(3-(((((S)-5-oxopyrrolidin-2-yl)methyl)amino)methyl)-1H-pyrazolo[3,4-b]pyridin-5-yl)-[1,1′-biphenyl]-3-yl)-2-methoxypyridin-3-yl)methyl)amino)methyl)pyrrolidin-2-one;
(R)-5-((((6-(2,2′-dichloro-3′-(3-(((((S)-5-oxopyrrolidin-2-yl)methyl)amino)methyl)-1H-pyrazolo[3,4-b]pyridin-5-yl)-[1,1′-biphenyl]-3-yl)-2-methoxypyridin-3-yl)methyl)amino)methyl)pyrrolidin-2-one;
(S)-5-((((6-(2,2′-dichloro-3′-(3-(((((R)-5-oxopyrrolidin-2-yl)methyl)amino)methyl)-1H-pyrazolo[3,4-b]pyridin-5-yl)-[1,1′-biphenyl]-3-yl)-2-methoxypyridin-3-yl)methyl)amino)methyl)pyrrolidin-2-one;
(R)-5-((((6-(2,2′-dichloro-3′-(3-(((((R)-5-oxopyrrolidin-2-yl)methyl)amino)methyl)-1H-pyrazolo[3,4-b]pyridin-5-yl)-[1,1′-biphenyl]-3-yl)-2-methoxypyridin-3-yl)methyl)amino)methyl)pyrrolidin-2-one;
(S)-5-((((6-(2,2′-dichloro-3′-((S)-5-((((S)-5-oxopyrrolidin-2-yl)methyl)amino)-5,6,7,8-tetrahydroquinolin-2-yl)-[1,1′-biphenyl]-3-yl)-2-methoxypyridin-3-yl)methyl)amino)methyl)pyrrolidin-2-one;
(S)-5-((((6-(2,2′-dichloro-3′-((R)-5-((((S)-5-oxopyrrolidin-2-yl)methyl)amino)-5,6,7,8-tetrahydroquinolin-2-yl)-[1,1′-biphenyl]-3-yl)-2-methoxypyridin-3-yl)methyl)amino)methyl)pyrrolidin-2-one;
(S)-5-((((6-(2,2′-dichloro-3′-((S)-5-((((R)-5-oxopyrrolidin-2-yl)methyl)amino)-5,6,7,8-tetrahydroquinolin-2-yl)-[1,1′-biphenyl]-3-yl)-2-methoxypyridin-3-yl)methyl)amino)methyl)pyrrolidin-2-one;
(S)-5-((((6-(2,2′-dichloro-3′-((R)-5-((((R)-5-oxopyrrolidin-2-yl)methyl)amino)-5,6,7,8-tetrahydroquinolin-2-yl)-[1,1′-biphenyl]-3-yl)-2-methoxypyridin-3-yl)methyl)amino)methyl)pyrrolidin-2-one;
(R)-5-((((6-(2,2′-dichloro-3′-((S)-5-((((S)-5-oxopyrrolidin-2-yl)methyl)amino)-5,6,7,8-tetrahydroquinolin-2-yl)-[1,1′-biphenyl]-3-yl)-2-methoxypyridin-3-yl)methyl)amino)methyl)pyrrolidin-2-one;
(R)-5-((((6-(2,2′-dichloro-3′-((R)-5-((((S)-5-oxopyrrolidin-2-yl)methyl)amino)-5,6,7,8-tetrahydroquinolin-2-yl)-[1,1′-biphenyl]-3-yl)-2-methoxypyridin-3-yl)methyl)amino)methyl)pyrrolidin-2-one;
(R)-5-((((6-(2,2′-dichloro-3′-((S)-5-((((R)-5-oxopyrrolidin-2-yl)methyl)amino)-5,6,7,8-tetrahydroquinolin-2-yl)-[1,1′-biphenyl]-3-yl)-2-methoxypyridin-3-yl)methyl)amino)methyl)pyrrolidin-2-one;
(R)-5-((((6-(2,2′-dichloro-3′-((R)-5-((((R)-5-oxopyrrolidin-2-yl)methyl)amino)-5,6,7,8-tetrahydroquinolin-2-yl)-[1,1′-biphenyl]-3-yl)-2-methoxypyridin-3-yl)methyl)amino)methyl)pyrrolidin-2-one;
6-(2,2′-dichloro-3′-(6-methoxy-5-(((((S)-5-oxopyrrolidin-2-yl)methyl)amino)methyl)pyridin-2-yl)-[1,1′-biphenyl]-3-yl)-2-((((S)-2-hydroxypropyl)(methyl)amino)methyl)-3-methylpyrrolo[2,1-f][1,2,4]triazin-4(3H)-one;
6-(2,2′-dichloro-3′-(6-methoxy-5-(((((R)-5-oxopyrrolidin-2-yl)methyl)amino)methyl)pyridin-2-yl)-[1,1′-biphenyl]-3-yl)-2-((((S)-2-hydroxypropyl)(methyl)amino)methyl)-3-methylpyrrolo[2,1-f][1,2,4]triazin-4(3H)-one;
6-(2,2′-dichloro-3′-(6-methoxy-5-(((((S)-5-oxopyrrolidin-2-yl)methyl)amino)methyl)pyridin-2-yl)-[1,1′-biphenyl]-3-yl)-2-((((R)-2-hydroxypropyl)(methyl)amino)methyl)-3-methylpyrrolo[2,1-f][1,2,4]triazin-4(3H)-one;
6-(2,2′-dichloro-3′-(6-methoxy-5-(((((R)-5-oxopyrrolidin-2-yl)methyl)amino)methyl)pyridin-2-yl)-[1,1′-biphenyl]-3-yl)-2-((((R)-2-hydroxypropyl)(methyl)amino)methyl)-3-methylpyrrolo[2,1-f][1,2,4]triazin-4(3H)-one;
(S)-6-(2,2′-dichloro-3′-(6-methoxy-5-((((5-oxopyrrolidin-2-yl)methyl)amino)methyl)pyridin-2-yl)-[1,1′-biphenyl]-3-yl)-2-(((3-fluoropropyl)amino)methyl)-3-methylpyrrolo[2,1-f][1,2,4]triazin-4(3H)-one;
(R)-6-(2,2′-dichloro-3′-(6-methoxy-5-((((5-oxopyrrolidin-2-yl)methyl)amino)methyl)pyridin-2-yl)-[1,1′-biphenyl]-3-yl)-2-(((3-fluoropropyl)amino)methyl)-3-methylpyrrolo[2,1-f][1,2,4]triazin-4(3H)-one;
(S)—N-(2-aminoethyl)-6-(2,2′-dichloro-3′-(6-methoxy-5-((((5-oxopyrrolidin-2-yl)methyl)amino)methyl)pyridin-2-yl)-[1,1′-biphenyl]-3-yl)imidazo[1,2-b]pyridazine-2-carboxamide;
(R)—N-(2-aminoethyl)-6-(2,2′-dichloro-3′-(6-methoxy-5-((((5-oxopyrrolidin-2-yl)methyl)amino)methyl)pyridin-2-yl)-[1,1′-biphenyl]-3-yl)imidazo[1,2-b]pyridazine-2-carboxamide;
N—((S)-2-aminopropyl)-6-(2,2′-dichloro-3′-(6-methoxy-5-(((((S)-5-oxopyrrolidin-2-yl)methyl)amino)methyl)pyridin-2-yl)-[1,1′-biphenyl]-3-yl)imidazo[1,2-b]pyridazine-2-carboxamide;
N—((R)-2-aminopropyl)-6-(2,2′-dichloro-3′-(6-methoxy-5-(((((S)-5-oxopyrrolidin-2-yl)methyl)amino)methyl)pyridin-2-yl)-[1,1′-biphenyl]-3-yl)imidazo[1,2-b]pyridazine-2-carboxamide;
N—((S)-2-aminopropyl)-6-(2,2′-dichloro-3′-(6-methoxy-5-(((((R)-5-oxopyrrolidin-2-yl)methyl)amino)methyl)pyridin-2-yl)-[1,1′-biphenyl]-3-yl)imidazo[1,2-b]pyridazine-2-carboxamide;
N—((R)-2-aminopropyl)-6-(2,2′-dichloro-3′-(6-methoxy-5-(((((R)-5-oxopyrrolidin-2-yl)methyl)amino)methyl)pyridin-2-yl)-[1,1′-biphenyl]-3-yl)imidazo[1,2-b]pyridazine-2-carboxamide;
(S)—N-(2-(1H-imidazol-4-yl)ethyl)-6-(2,2′-dichloro-3′-(6-methoxy-5-((((5-oxopyrrolidin-2-yl)methyl)amino)methyl)pyridin-2-yl)-[1,1′-biphenyl]-3-yl)imidazo[1,2-b]pyridazine-2-carboxamide;
(R)—N-(2-(1H-imidazol-4-yl)ethyl)-6-(2,2′-dichloro-3′-(6-methoxy-5-((((5-oxopyrrolidin-2-yl)methyl)amino)methyl)pyridin-2-yl)-[1,1′-biphenyl]-3-yl)imidazo[1,2-b]pyridazine-2-carboxamide;
8-(2,2′-dichloro-3′-(2-(((((S)-5-oxopyrrolidin-2-yl)methyl)amino)methyl)thiazol-5-yl)-[1,1′-biphenyl]-3-yl)-3-(((((S)-5-oxopyrrolidin-2-yl)methyl)amino)methyl)-4H-pyrido[1,2-a]pyrimidin-4-one;
8-(2,2′-dichloro-3′-(2-(((((R)-5-oxopyrrolidin-2-yl)methyl)amino)methyl)thiazol-5-yl)-[1,1′-biphenyl]-3-yl)-3-(((((S)-5-oxopyrrolidin-2-yl)methyl)amino)methyl)-4H-pyrido[1,2-a]pyrimidin-4-one;
8-(2,2′-dichloro-3′-(2-(((((S)-5-oxopyrrolidin-2-yl)methyl)amino)methyl)thiazol-5-yl)-[1,1′-biphenyl]-3-yl)-3-(((((R)-5-oxopyrrolidin-2-yl)methyl)amino)methyl)-4H-pyrido[1,2-a]pyrimidin-4-one;
8-(2,2′-dichloro-3′-(2-(((((R)-5-oxopyrrolidin-2-yl)methyl)amino)methyl)thiazol-5-yl)-[1,1′-biphenyl]-3-yl)-3-(((((R)-5-oxopyrrolidin-2-yl)methyl)amino)methyl)-4H-pyrido[1,2-a]pyrimidin-4-one;
8-(2,2′-dichloro-3′-(2-(((((S)-5-oxopyrrolidin-2-yl)methyl)amino)methyl)thiazol-4-yl)-[1,1′-biphenyl]-3-yl)-3-(((((S)-5-oxopyrrolidin-2-yl)methyl)amino)methyl)-4H-pyrido[1,2-a]pyrimidin-4-one;
8-(2,2′-dichloro-3′-(2-(((((R)-5-oxopyrrolidin-2-yl)methyl)amino)methyl)thiazol-4-yl)-[1,1′-biphenyl]-3-yl)-3-(((((S)-5-oxopyrrolidin-2-yl)methyl)amino)methyl)-4H-pyrido[1,2-a]pyrimidin-4-one;
8-(2,2′-dichloro-3′-(2-(((((S)-5-oxopyrrolidin-2-yl)methyl)amino)methyl)thiazol-4-yl)-[1,1′-biphenyl]-3-yl)-3-(((((R)-5-oxopyrrolidin-2-yl)methyl)amino)methyl)-4H-pyrido[1,2-a]pyrimidin-4-one;
8-(2,2′-dichloro-3′-(2-(((((R)-5-oxopyrrolidin-2-yl)methyl)amino)methyl)thiazol-4-yl)-[1,1′-biphenyl]-3-yl)-3-(((((R)-5-oxopyrrolidin-2-yl)methyl)amino)methyl)-4H-pyrido[1,2-a]pyrimidin-4-one;
(S)-5-((((6-(2,2′-dichloro-3′-(6-(((S)-5-oxopyrrolidin-2-yl)methyl)-5,6,7,8-tetrahydropyrido[4,3-d]pyrimidin-2-yl)-[1,1′-biphenyl]-3-yl)-2-methoxypyridin-3-yl)methyl)amino)methyl)pyrrolidin-2-one;
(R)-5-((((6-(2,2′-dichloro-3′-(6-(((S)-5-oxopyrrolidin-2-yl)methyl)-5,6,7,8-tetrahydropyrido[4,3-d]pyrimidin-2-yl)-[1,1′-biphenyl]-3-yl)-2-methoxypyridin-3-yl)methyl)amino)methyl)pyrrolidin-2-one;
(S)-5-((((6-(2,2′-dichloro-3′-(6-(((R)-5-oxopyrrolidin-2-yl)methyl)-5,6,7,8-tetrahydropyrido[4,3-d]pyrimidin-2-yl)-[1,1′-biphenyl]-3-yl)-2-methoxypyridin-3-yl)methyl)amino)methyl)pyrrolidin-2-one;
(R)-5-((((6-(2,2′-dichloro-3′-(6-(((R)-5-oxopyrrolidin-2-yl)methyl)-5,6,7,8-tetrahydropyrido[4,3-d]pyrimidin-2-yl)-[1,1′-biphenyl]-3-yl)-2-methoxypyridin-3-yl)methyl)amino)methyl)pyrrolidin-2-one;
(S)-5-((((6-(2,2′-dichloro-3′-(1-methyl-3-(((((S)-tetrahydrofuran-2-yl)methyl)amino)methyl)-1H-pyrrolo[2,3-b]pyridin-6-yl)-[1,1′-biphenyl]-3-yl)-2-methoxypyridin-3-yl)methyl)amino)methyl)pyrrolidin-2-one;
(R)-5-((((6-(2,2′-dichloro-3′-(1-methyl-3-(((((S)-tetrahydrofuran-2-yl)methyl)amino)methyl)-1H-pyrrolo[2,3-b]pyridin-6-yl)-[1,1′-biphenyl]-3-yl)-2-methoxypyridin-3-yl)methyl)amino)methyl)pyrrolidin-2-one;
(S)-5-((((6-(2,2′-dichloro-3′-(1-methyl-3-(((((R)-tetrahydrofuran-2-yl)methyl)amino)methyl)-1H-pyrrolo[2,3-b]pyridin-6-yl)-[1,1′-biphenyl]-3-yl)-2-methoxypyridin-3-yl)methyl)amino)methyl)pyrrolidin-2-one;
(R)-5-((((6-(2,2′-dichloro-3′-(1-methyl-3-(((((R)-tetrahydrofuran-2-yl)methyl)amino)methyl)-1H-pyrrolo[2,3-b]pyridin-6-yl)-[1,1′-biphenyl]-3-yl)-2-methoxypyridin-3-yl)methyl)amino)methyl)pyrrolidin-2-one;
(S)-5-((((6-(2,2′-dichloro-3′-(3-((((S)-2-methoxypropyl)amino)methyl)-1-methyl-1H-pyrrolo[2,3-b]pyridin-6-yl)-[1,1′-biphenyl]-3-yl)-2-methoxypyridin-3-yl)methyl)amino)methyl)pyrrolidin-2-one;
(S)-5-((((6-(2,2′-dichloro-3′-(3-((((R)-2-methoxypropyl)amino)methyl)-1-methyl-1H-pyrrolo[2,3-b]pyridin-6-yl)-[1,1′-biphenyl]-3-yl)-2-methoxypyridin-3-yl)methyl)amino)methyl)pyrrolidin-2-one;
(R)-5-((((6-(2,2′-dichloro-3′-(3-((((S)-2-methoxypropyl)amino)methyl)-1-methyl-1H-pyrrolo[2,3-b]pyridin-6-yl)-[1,1′-biphenyl]-3-yl)-2-methoxypyridin-3-yl)methyl)amino)methyl)pyrrolidin-2-one;
(R)-5-((((6-(2,2′-dichloro-3′-(3-((((R)-2-methoxypropyl)amino)methyl)-1-methyl-1H-pyrrolo[2,3-b]pyridin-6-yl)-[1,1′-biphenyl]-3-yl)-2-methoxypyridin-3-yl)methyl)amino)methyl)pyrrolidin-2-one;
(S)-2-(((2-oxaspiro[3.3]heptan-6-yl)amino)methyl)-6-(2,2′-dichloro-3′-(6-methoxy-5-((((5-oxopyrrolidin-2-yl)methyl)amino)methyl)pyridin-2-yl)-[1,1′-biphenyl]-3-yl)-3-methylpyrrolo[2,1-f][1,2,4]triazin-4(3H)-one;
(R)-2-(((2-oxaspiro[3.3]heptan-6-yl)amino)methyl)-6-(2,2′-dichloro-3′-(6-methoxy-5-((((5-oxopyrrolidin-2-yl)methyl)amino)methyl)pyridin-2-yl)-[1,1′-biphenyl]-3-yl)-3-methylpyrrolo[2,1-f][1,2,4]triazin-4(3H)-one;
(S)-2-((2-oxa-6-azaspiro[3.3]heptan-6-yl)methyl)-6-(2,2′-dichloro-3′-(6-methoxy-5-((((5-oxopyrrolidin-2-yl)methyl)amino)methyl)pyridin-2-yl)-[1,1′-biphenyl]-3-yl)-3-methylpyrrolo[2,1-f][1,2,4]triazin-4(3H)-one;
(R)-2-((2-oxa-6-azaspiro[3.3]heptan-6-yl)methyl)-6-(2,2′-dichloro-3′-(6-methoxy-5-((((5-oxopyrrolidin-2-yl)methyl)amino)methyl)pyridin-2-yl)-[1,1′-biphenyl]-3-yl)-3-methylpyrrolo[2,1-f][1,2,4]triazin-4(3H)-one;
(S)-6-(2,2′-dichloro-3′-(6-methoxy-5-((((5-oxopyrrolidin-2-yl)methyl)amino)methyl)pyridin-2-yl)-[1,1′-biphenyl]-3-yl)-3-methyl-2-((6-oxo-2,5-diazaspiro[3.4]octan-2-yl)methyl)pyrrolo[2,1-f][1,2,4]triazin-4(3H)-one;
(R)-6-(2,2′-dichloro-3′-(6-methoxy-5-((((5-oxopyrrolidin-2-yl)methyl)amino)methyl)pyridin-2-yl)-[1,1′-biphenyl]-3-yl)-3-methyl-2-((6-oxo-2,5-diazaspiro[3.4]octan-2-yl)methyl)pyrrolo[2,1-f][1,2,4]triazin-4(3H)-one;
6-(2,2′-dichloro-3′-(6-methoxy-5-(((((S)-5-oxopyrrolidin-2-yl)methyl)amino)methyl)pyridin-2-yl)-[1,1′-biphenyl]-3-yl)-3-methyl-2-((((2s,4r)-6-oxo-5-azaspiro[3.4]octan-2-yl)amino)methyl)pyrrolo[2,1-f][1,2,4]triazin-4(3H)-one;
6-(2,2′-dichloro-3′-(6-methoxy-5-(((((S)-5-oxopyrrolidin-2-yl)methyl)amino)methyl)pyridin-2-yl)-[1,1′-biphenyl]-3-yl)-3-methyl-2-((((2r,4s)-6-oxo-5-azaspiro[3.4]octan-2-yl)amino)methyl)pyrrolo[2,1-f][1,2,4]triazin-4(3H)-one;
6-(2,2′-dichloro-3′-(6-methoxy-5-(((((R)-5-oxopyrrolidin-2-yl)methyl)amino)methyl)pyridin-2-yl)-[1,1′-biphenyl]-3-yl)-3-methyl-2-((((2s,4r)-6-oxo-5-azaspiro[3.4]octan-2-yl)amino)methyl)pyrrolo[2,1-f][1,2,4]triazin-4(3H)-one;
6-(2,2′-dichloro-3′-(6-methoxy-5-(((((R)-5-oxopyrrolidin-2-yl)methyl)amino)methyl)pyridin-2-yl)-[1,1′-biphenyl]-3-yl)-3-methyl-2-((((2r,4s)-6-oxo-5-azaspiro[3.4]octan-2-yl)amino)methyl)pyrrolo[2,1-f][1,2,4]triazin-4(3H)-one;
6-(2,2′-dichloro-3′-(6-methoxy-5-(((((S)-5-oxopyrrolidin-2-yl)methyl)amino)methyl)pyridin-2-yl)-[1,1′-biphenyl]-3-yl)-2-(((((R)-4,4-difluoro-5-oxopyrrolidin-2-yl)methyl)amino)methyl)-3-methylpyrrolo[2,1-f][1,2,4]triazin-4(3H)-one;
6-(2,2′-dichloro-3′-(6-methoxy-5-(((((S)-5-oxopyrrolidin-2-yl)methyl)amino)methyl)pyridin-2-yl)-[1,1′-biphenyl]-3-yl)-2-(((((S)-4,4-difluoro-5-oxopyrrolidin-2-yl)methyl)amino)methyl)-3-methylpyrrolo[2,1-f][1,2,4]triazin-4(3H)-one;
6-(2,2′-dichloro-3′-(6-methoxy-5-(((((R)-5-oxopyrrolidin-2-yl)methyl)amino)methyl)pyridin-2-yl)-[1,1′-biphenyl]-3-yl)-2-(((((R)-4,4-difluoro-5-oxopyrrolidin-2-yl)methyl)amino)methyl)-3-methylpyrrolo[2,1-f][1,2,4]triazin-4(3H)-one;
6-(2,2′-dichloro-3′-(6-methoxy-5-(((((R)-5-oxopyrrolidin-2-yl)methyl)amino)methyl)pyridin-2-yl)-[1,1′-biphenyl]-3-yl)-2-(((((S)-4,4-difluoro-5-oxopyrrolidin-2-yl)methyl)amino)methyl)-3-methylpyrrolo[2,1-f][1,2,4]triazin-4(3H)-one;
6-(2,2′-dichloro-3′-(6-methoxy-5-(((((S)-5-oxopyrrolidin-2-yl)methyl)amino)methyl)pyridin-2-yl)-[1,1′-biphenyl]-3-yl)-3-methyl-2-(((2-((R)-5-oxopyrrolidin-2-yl)ethyl)amino)methyl)pyrrolo[2,1-f][1,2,4]triazin-4(3H)-one;
6-(2,2′-dichloro-3′-(6-methoxy-5-(((((S)-5-oxopyrrolidin-2-yl)methyl)amino)methyl)pyridin-2-yl)-[1,1′-biphenyl]-3-yl)-3-methyl-2-(((2-((S)-5-oxopyrrolidin-2-yl)ethyl)amino)methyl)pyrrolo[2,1-f][1,2,4]triazin-4(3H)-one;
6-(2,2′-dichloro-3′-(6-methoxy-5-(((((R)-5-oxopyrrolidin-2-yl)methyl)amino)methyl)pyridin-2-yl)-[1,1′-biphenyl]-3-yl)-3-methyl-2-(((2-((R)-5-oxopyrrolidin-2-yl)ethyl)amino)methyl)pyrrolo[2,1-f][1,2,4]triazin-4(3H)-one;
6-(2,2′-dichloro-3′-(6-methoxy-5-(((((R)-5-oxopyrrolidin-2-yl)methyl)amino)methyl)pyridin-2-yl)-[1,1′-biphenyl]-3-yl)-3-methyl-2-(((2-((S)-5-oxopyrrolidin-2-yl)ethyl)amino)methyl)pyrrolo[2,1-f][1,2,4]triazin-4(3H)-one;
(S)-5-((((6-(2,2′-dichloro-3′-(1-methyl-3-(((((S)-5-oxopyrrolidin-2-yl)methyl)amino)methyl)-1H-indol-6-yl)-[1,1′-biphenyl]-3-yl)-2-methoxypyridin-3-yl)methyl)amino)methyl)pyrrolidin-2-one;
(R)-5-((((6-(2,2′-dichloro-3′-(1-methyl-3-(((((S)-5-oxopyrrolidin-2-yl)methyl)amino)methyl)-1H-indol-6-yl)-[1,1′-biphenyl]-3-yl)-2-methoxypyridin-3-yl)methyl)amino)methyl)pyrrolidin-2-one;
(S)-5-((((6-(2,2′-dichloro-3′-(1-methyl-3-(((((R)-5-oxopyrrolidin-2-yl)methyl)amino)methyl)-1H-indol-6-yl)-[1,1′-biphenyl]-3-yl)-2-methoxypyridin-3-yl)methyl)amino)methyl)pyrrolidin-2-one;
(R)-5-((((6-(2,2′-dichloro-3′-(1-methyl-3-(((((R)-5-oxopyrrolidin-2-yl)methyl)amino)methyl)-1H-indol-6-yl)-[1,1′-biphenyl]-3-yl)-2-methoxypyridin-3-yl)methyl)amino)methyl)pyrrolidin-2-one;
8-(2,2′-dichloro-3′-(1-methyl-3-(((((S)-5-oxopyrrolidin-2-yl)methyl)amino)methyl)-1H-indol-6-yl)-[1,1′-biphenyl]-3-yl)-3-(((((S)-5-oxopyrrolidin-2-yl)methyl)amino)methyl)-4H-pyrido[1,2-a]pyrimidin-4-one;
8-(2,2′-dichloro-3′-(1-methyl-3-(((((R)-5-oxopyrrolidin-2-yl)methyl)amino)methyl)-1H-indol-6-yl)-[1,1′-biphenyl]-3-yl)-3-(((((S)-5-oxopyrrolidin-2-yl)methyl)amino)methyl)-4H-pyrido[1,2-a]pyrimidin-4-one;
8-(2,2′-dichloro-3′-(1-methyl-3-(((((S)-5-oxopyrrolidin-2-yl)methyl)amino)methyl)-1H-indol-6-yl)-[1,1′-biphenyl]-3-yl)-3-(((((R)-5-oxopyrrolidin-2-yl)methyl)amino)methyl)-4H-pyrido[1,2-a]pyrimidin-4-one;
8-(2,2′-dichloro-3′-(1-methyl-3-(((((R)-5-oxopyrrolidin-2-yl)methyl)amino)methyl)-1H-indol-6-yl)-[1,1′-biphenyl]-3-yl)-3-(((((R)-5-oxopyrrolidin-2-yl)methyl)amino)methyl)-4H-pyrido[1,2-a]pyrimidin-4-one;
6-(2,2′-dichloro-3′-(6-methoxy-5-(((((S)-5-oxopyrrolidin-2-yl)methyl)amino)methyl)pyridin-2-yl)-[1,1′-biphenyl]-3-yl)-2-(2-((((S)-5-oxopyrrolidin-2-yl)methyl)amino)ethyl)-2,3-dihydro-1H-pyrrolo[3,4-c]pyridin-1-one;
6-(2,2′-dichloro-3′-(6-methoxy-5-(((((R)-5-oxopyrrolidin-2-yl)methyl)amino)methyl)pyridin-2-yl)-[1,1′-biphenyl]-3-yl)-2-(2-((((S)-5-oxopyrrolidin-2-yl)methyl)amino)ethyl)-2,3-dihydro-1H-pyrrolo[3,4-c]pyridin-1-one;
6-(2,2′-dichloro-3′-(6-methoxy-5-(((((S)-5-oxopyrrolidin-2-yl)methyl)amino)methyl)pyridin-2-yl)-[1,1′-biphenyl]-3-yl)-2-(2-((((R)-5-oxopyrrolidin-2-yl)methyl)amino)ethyl)-2,3-dihydro-1H-pyrrolo[3,4-c]pyridin-1-one;
6-(2,2′-dichloro-3′-(6-methoxy-5-(((((R)-5-oxopyrrolidin-2-yl)methyl)amino)methyl)pyridin-2-yl)-[1,1′-biphenyl]-3-yl)-2-(2-((((R)-5-oxopyrrolidin-2-yl)methyl)amino)ethyl)-2,3-dihydro-1H-pyrrolo[3,4-c]pyridin-1-one;
(S)-5-((((6-(2,2′-dichloro-3′-(2-(2-hydroxyethyl)-8-methoxy-1,2,3,4-tetrahydroisoquinolin-6-yl)-[1,1′-biphenyl]-3-yl)-2-methoxypyridin-3-yl)methyl)amino)methyl)pyrrolidin-2-one;
(R)-5-((((6-(2,2′-dichloro-3′-(2-(2-hydroxyethyl)-8-methoxy-1,2,3,4-tetrahydroisoquinolin-6-yl)-[1,1′-biphenyl]-3-yl)-2-methoxypyridin-3-yl)methyl)amino)methyl)pyrrolidin-2-one;
(S)-5-((((6-(2,2′-dichloro-3′-((R)-5-((((S)-5-oxopyrrolidin-2-yl)methyl)amino)-5,6,7,8-tetrahydroquinolin-2-yl)-[1,1′-biphenyl]-3-yl)-2-methoxypyridin-3-yl)methyl)amino)methyl)pyrrolidin-2-one;
(S)-5-((((6-(2,2′-dichloro-3′-((S)-5-((((R)-5-oxopyrrolidin-2-yl)methyl)amino)-5,6,7,8-tetrahydroquinolin-2-yl)-[1,1′-biphenyl]-3-yl)-2-methoxypyridin-3-yl)methyl)amino)methyl)pyrrolidin-2-one;
(S)-5-((((6-(2,2′-dichloro-3′-((R)-5-((((R)-5-oxopyrrolidin-2-yl)methyl)amino)-5,6,7,8-tetrahydroquinolin-2-yl)-[1,1′-biphenyl]-3-yl)-2-methoxypyridin-3-yl)methyl)amino)methyl)pyrrolidin-2-one;
(S)-5-((((6-(2,2′-dichloro-3′-((S)-5-((((S)-5-oxopyrrolidin-2-yl)methyl)amino)-5,6,7,8-tetrahydroquinolin-2-yl)-[1,1′-biphenyl]-3-yl)-2-methoxypyridin-3-yl)methyl)amino)methyl)pyrrolidin-2-one;
(R)-5-((((6-(2,2′-dichloro-3′-((R)-5-((((S)-5-oxopyrrolidin-2-yl)methyl)amino)-5,6,7,8-tetrahydroquinolin-2-yl)-[1,1′-biphenyl]-3-yl)-2-methoxypyridin-3-yl)methyl)amino)methyl)pyrrolidin-2-one;
(R)-5-((((6-(2,2′-dichloro-3′-((S)-5-((((R)-5-oxopyrrolidin-2-yl)methyl)amino)-5,6,7,8-tetrahydroquinolin-2-yl)-[1,1′-biphenyl]-3-yl)-2-methoxypyridin-3-yl)methyl)amino)methyl)pyrrolidin-2-one;
(R)-5-((((6-(2,2′-dichloro-3′-((R)-5-((((R)-5-oxopyrrolidin-2-yl)methyl)amino)-5,6,7,8-tetrahydroquinolin-2-yl)-[1,1′-biphenyl]-3-yl)-2-methoxypyridin-3-yl)methyl)amino)methyl)pyrrolidin-2-one;
(R)-5-((((6-(2,2′-dichloro-3′-((S)-5-((((S)-5-oxopyrrolidin-2-yl)methyl)amino)-5,6,7,8-tetrahydroquinolin-2-yl)-[1,1′-biphenyl]-3-yl)-2-methoxypyridin-3-yl)methyl)amino)methyl)pyrrolidin-2-one;
(S)-2-(((1-acetylpiperidin-4-yl)amino)methyl)-6-(2,2′-dichloro-3′-(6-methoxy-5-((((5-oxopyrrolidin-2-yl)methyl)amino)methyl)pyridin-2-yl)-[1,1′-biphenyl]-3-yl)-3-methylpyrrolo[2,1-f][1,2,4]triazin-4(3H)-one;
(R)-2-(((1-acetylpiperidin-4-yl)amino)methyl)-6-(2,2′-dichloro-3′-(6-methoxy-5-((((5-oxopyrrolidin-2-yl)methyl)amino)methyl)pyridin-2-yl)-[1,1′-biphenyl]-3-yl)-3-methylpyrrolo[2,1-f][1,2,4]triazin-4(3H)-one;
(S)-6-(2,2′-dichloro-3′-(6-methoxy-5-((((5-oxopyrrolidin-2-yl)methyl)amino)methyl)pyridin-2-yl)-[1,1′-biphenyl]-3-yl)-3-methyl-2-(((2-(2-oxopyrrolidin-1-yl)ethyl)amino)methyl)pyrrolo[2,1-f][1,2,4]triazin-4(3H)-one;
(R)-6-(2,2′-dichloro-3′-(6-methoxy-5-((((5-oxopyrrolidin-2-yl)methyl)amino)methyl)pyridin-2-yl)-[1,1′-biphenyl]-3-yl)-3-methyl-2-(((2-(2-oxopyrrolidin-1-yl)ethyl)amino)methyl)pyrrolo[2,1-f][1,2,4]triazin-4(3H)-one;
(S)-5-((((6-(2,2′-dichloro-3′-(2-(((((S)-5-oxopyrrolidin-2-yl)methyl)amino)methyl)furo[2,3-b]pyridin-5-yl)-[1,1′-biphenyl]-3-yl)-2-methoxypyridin-3-yl)methyl)amino)methyl)pyrrolidin-2-one;
(R)-5-((((6-(2,2′-dichloro-3′-(2-(((((S)-5-oxopyrrolidin-2-yl)methyl)amino)methyl)furo[2,3-b]pyridin-5-yl)-[1,1′-biphenyl]-3-yl)-2-methoxypyridin-3-yl)methyl)amino)methyl)pyrrolidin-2-one;
(S)-5-((((6-(2,2′-dichloro-3′-(2-(((((R)-5-oxopyrrolidin-2-yl)methyl)amino)methyl)furo[2,3-b]pyridin-5-yl)-[1,1′-biphenyl]-3-yl)-2-methoxypyridin-3-yl)methyl)amino)methyl)pyrrolidin-2-one;
(R)-5-((((6-(2,2′-dichloro-3′-(2-(((((R)-5-oxopyrrolidin-2-yl)methyl)amino)methyl)furo[2,3-b]pyridin-5-yl)-[1,1′-biphenyl]-3-yl)-2-methoxypyridin-3-yl)methyl)amino)methyl)pyrrolidin-2-one;
(S)-2-((6-acetyl-2,6-diazaspiro[3.3]heptan-2-yl)methyl)-6-(2,2′-dichloro-3′-(6-methoxy-5-((((5-oxopyrrolidin-2-yl)methyl)amino)methyl)pyridin-2-yl)-[1,1′-biphenyl]-3-yl)-3-methylpyrrolo[2,1-f][1,2,4]triazin-4(3H)-one;
(R)-2-((6-acetyl-2,6-diazaspiro[3.3]heptan-2-yl)methyl)-6-(2,2′-dichloro-3′-(6-methoxy-5-((((5-oxopyrrolidin-2-yl)methyl)amino)methyl)pyridin-2-yl)-[1,1′-biphenyl]-3-yl)-3-methylpyrrolo[2,1-f][1,2,4]triazin-4(3H)-one;
(S)-6-(2,2′-dichloro-3′-(6-methoxy-5-((((5-oxopyrrolidin-2-yl)methyl)amino)methyl)pyridin-2-yl)-[1,1′-biphenyl]-3-yl)-2-((3-(2-hydroxypropan-2-yl)azetidin-1-yl)methyl)-3-methylpyrrolo[2,1-f][1,2,4]triazin-4(3H)-one;
(R)-6-(2,2′-dichloro-3′-(6-methoxy-5-((((5-oxopyrrolidin-2-yl)methyl)amino)methyl)pyridin-2-yl)-[1,1′-biphenyl]-3-yl)-2-((3-(2-hydroxypropan-2-yl)azetidin-1-yl)methyl)-3-methylpyrrolo[2,1-f][1,2,4]triazin-4(3H)-one;
(S)-6-(2,2′-dichloro-3′-(6-methoxy-5-((((5-oxopyrrolidin-2-yl)methyl)amino)methyl)pyridin-2-yl)-[1,1′-biphenyl]-3-yl)-2-(((2-fluoro-2-methylpropyl)amino)methyl)-3-methylpyrrolo[2,1-f][1,2,4]triazin-4(3H)-one;
(R)-6-(2,2′-dichloro-3′-(6-methoxy-5-((((5-oxopyrrolidin-2-yl)methyl)amino)methyl)pyridin-2-yl)-[1,1′-biphenyl]-3-yl)-2-(((2-fluoro-2-methylpropyl)amino)methyl)-3-methylpyrrolo[2,1-f][1,2,4]triazin-4(3H)-one;
(S)-6-(2,2′-dichloro-3′-(6-methoxy-5-((((5-oxopyrrolidin-2-yl)methyl)amino)methyl)pyridin-2-yl)-[1,1′-biphenyl]-3-yl)-2-((((1-fluorocyclopropyl)methyl)amino)methyl)-3-methylpyrrolo[2,1-f][1,2,4]triazin-4(3H)-one;
(R)-6-(2,2′-dichloro-3′-(6-methoxy-5-((((5-oxopyrrolidin-2-yl)methyl)amino)methyl)pyridin-2-yl)-[1,1′-biphenyl]-3-yl)-2-((((1-fluorocyclopropyl)methyl)amino)methyl)-3-methylpyrrolo[2,1-f][1,2,4]triazin-4(3H)-one;
6-(2,2′-dichloro-3′-(4-chloro-6-methoxy-5-(((((S)-5-oxopyrrolidin-2-yl)methyl)amino)methyl)pyridin-2-yl)-[1,1′-biphenyl]-3-yl)-2-((((1r,3r)-3-hydroxy-3-methylcyclobutyl)amino)methyl)-3-methylpyrrolo[2,1-f][1,2,4]triazin-4(3H)-one;
6-(2,2′-dichloro-3′-(4-chloro-6-methoxy-5-(((((S)-5-oxopyrrolidin-2-yl)methyl)amino)methyl)pyridin-2-yl)-[1,1′-biphenyl]-3-yl)-2-((((1s,3s)-3-hydroxy-3-methylcyclobutyl)amino)methyl)-3-methylpyrrolo[2,1-f][1,2,4]triazin-4(3H)-one;
6-(2,2′-dichloro-3′-(4-chloro-6-methoxy-5-(((((R)-5-oxopyrrolidin-2-yl)methyl)amino)methyl)pyridin-2-yl)-[1,1′-biphenyl]-3-yl)-2-((((1r,3r)-3-hydroxy-3-methylcyclobutyl)amino)methyl)-3-methylpyrrolo[2,1-f][1,2,4]triazin-4(3H)-one;
6-(2,2′-dichloro-3′-(4-chloro-6-methoxy-5-(((((R)-5-oxopyrrolidin-2-yl)methyl)amino)methyl)pyridin-2-yl)-[1,1′-biphenyl]-3-yl)-2-((((1s,3s)-3-hydroxy-3-methylcyclobutyl)amino)methyl)-3-methylpyrrolo[2,1-f][1,2,4]triazin-4(3H)-one;
(S)-6-(2,2′-dichloro-3′-(6-methoxy-5-((((5-oxopyrrolidin-2-yl)methyl)amino)methyl)pyridin-2-yl)-[1,1′-biphenyl]-3-yl)-2-(((3,3-difluoropropyl)amino)methyl)-3-methylpyrrolo[2,1-f][1,2,4]triazin-4(3H)-one;
(R)-6-(2,2′-dichloro-3′-(6-methoxy-5-((((5-oxopyrrolidin-2-yl)methyl)amino)methyl)pyridin-2-yl)-[1,1′-biphenyl]-3-yl)-2-(((3,3-difluoropropyl)amino)methyl)-3-methylpyrrolo[2,1-f][1,2,4]triazin-4(3H)-one;
(S)-6-(2,2′-dichloro-3′-(6-methoxy-5-((((5-oxopyrrolidin-2-yl)methyl)amino)methyl)pyridin-2-yl)-[1,1′-biphenyl]-3-yl)-3-methyl-2-(((3,3,3-trifluoro-2,2-dimethylpropyl)amino)methyl)pyrrolo[2,1-f][1,2,4]triazin-4(3H)-one;
(R)-6-(2,2′-dichloro-3′-(6-methoxy-5-((((5-oxopyrrolidin-2-yl)methyl)amino)methyl)pyridin-2-yl)-[1,1′-biphenyl]-3-yl)-3-methyl-2-(((3,3,3-trifluoro-2,2-dimethylpropyl)amino)methyl)pyrrolo[2,1-f][1,2,4]triazin-4(3H)-one;
6-(2,2′-dichloro-3′-(6-methoxy-5-(((((S)-5-oxopyrrolidin-2-yl)methyl)amino)methyl)pyridin-2-yl)-[1,1′-biphenyl]-3-yl)-2-(2-((((S)-5-oxopyrrolidin-2-yl)methyl)amino)ethyl)-1,2-dihydro-3H-pyrrolo[3,4-c]pyridin-3-one;
6-(2,2′-dichloro-3′-(6-methoxy-5-(((((R)-5-oxopyrrolidin-2-yl)methyl)amino)methyl)pyridin-2-yl)-[1,1′-biphenyl]-3-yl)-2-(2-((((S)-5-oxopyrrolidin-2-yl)methyl)amino)ethyl)-1,2-dihydro-3H-pyrrolo[3,4-c]pyridin-3-one;
6-(2,2′-dichloro-3′-(6-methoxy-5-(((((S)-5-oxopyrrolidin-2-yl)methyl)amino)methyl)pyridin-2-yl)-[1,1′-biphenyl]-3-yl)-2-(2-((((R)-5-oxopyrrolidin-2-yl)methyl)amino)ethyl)-1,2-dihydro-3H-pyrrolo[3,4-c]pyridin-3-one;
6-(2,2′-dichloro-3′-(6-methoxy-5-(((((R)-5-oxopyrrolidin-2-yl)methyl)amino)methyl)pyridin-2-yl)-[1,1′-biphenyl]-3-yl)-2-(2-((((R)-5-oxopyrrolidin-2-yl)methyl)amino)ethyl)-1,2-dihydro-3H-pyrrolo[3,4-c]pyridin-3-one;
(S)-6-(2,2′-dichloro-3′-(6-methoxy-5-((((5-oxopyrrolidin-2-yl)methyl)amino)methyl)pyridin-2-yl)-[1,1′-biphenyl]-3-yl)-2-((6-hydroxy-2-azaspiro[3.3]heptan-2-yl)methyl)-3-methylpyrrolo[2,1-f][1,2,4]triazin-4(3H)-one;
(R)-6-(2,2′-dichloro-3′-(6-methoxy-5-((((5-oxopyrrolidin-2-yl)methyl)amino)methyl)pyridin-2-yl)-[1,1′-biphenyl]-3-yl)-2-((6-hydroxy-2-azaspiro[3.3]heptan-2-yl)methyl)-3-methylpyrrolo[2,1-f][1,2,4]triazin-4(3H)-one;
(S)-6-(2,2′-dichloro-3′-(6-methoxy-5-((((5-oxopyrrolidin-2-yl)methyl)amino)methyl)pyridin-2-yl)-[1,1′-biphenyl]-3-yl)-2-((3-hydroxy-3-methylazetidin-1-yl)methyl)-3-methylpyrrolo[2,1-f][1,2,4]triazin-4(3H)-one;
(R)-6-(2,2′-dichloro-3′-(6-methoxy-5-((((5-oxopyrrolidin-2-yl)methyl)amino)methyl)pyridin-2-yl)-[1,1′-biphenyl]-3-yl)-2-((3-hydroxy-3-methylazetidin-1-yl)methyl)-3-methylpyrrolo[2,1-f][1,2,4]triazin-4(3H)-one;
(S)-1-((6-(2,2′-dichloro-3′-(6-methoxy-5-((((5-oxopyrrolidin-2-yl)methyl)amino)methyl)pyridin-2-yl)-[1,1′-biphenyl]-3-yl)-3-methyl-4-oxo-3,4-dihydropyrrolo[2,1-f][1,2,4]triazin-2-yl)methyl)-3-methylazetidine-3-carbonitrile;
(R)-1-((6-(2,2′-dichloro-3′-(6-methoxy-5-((((5-oxopyrrolidin-2-yl)methyl)amino)methyl)pyridin-2-yl)-[1,1′-biphenyl]-3-yl)-3-methyl-4-oxo-3,4-dihydropyrrolo[2,1-f][1,2,4]triazin-2-yl)methyl)-3-methylazetidine-3-carbonitrile;
(S)-5-((((6-(2,2′-dichloro-3′-(4-methoxy-6-(((S)-5-oxopyrrolidin-2-yl)methyl)-5,6,7,8-tetrahydropyrido[4,3-d]pyrimidin-2-yl)-[1,1′-biphenyl]-3-yl)-2-methoxypyridin-3-yl)methyl)amino)methyl)pyrrolidin-2-one;
(R)-5-((((6-(2,2′-dichloro-3′-(4-methoxy-6-(((S)-5-oxopyrrolidin-2-yl)methyl)-5,6,7,8-tetrahydropyrido[4,3-d]pyrimidin-2-yl)-[1,1′-biphenyl]-3-yl)-2-methoxypyridin-3-yl)methyl)amino)methyl)pyrrolidin-2-one;
(S)-5-((((6-(2,2′-dichloro-3′-(4-methoxy-6-(((R)-5-oxopyrrolidin-2-yl)methyl)-5,6,7,8-tetrahydropyrido[4,3-d]pyrimidin-2-yl)-[1,1′-biphenyl]-3-yl)-2-methoxypyridin-3-yl)methyl)amino)methyl)pyrrolidin-2-one;
(R)-5-((((6-(2,2′-dichloro-3′-(4-methoxy-6-(((R)-5-oxopyrrolidin-2-yl)methyl)-5,6,7,8-tetrahydropyrido[4,3-d]pyrimidin-2-yl)-[1,1′-biphenyl]-3-yl)-2-methoxypyridin-3-yl)methyl)amino)methyl)pyrrolidin-2-one;
6-(2,2′-dichloro-3′-(6-methoxy-5-(((((S)-5-oxopyrrolidin-2-yl)methyl)amino)methyl)pyridin-2-yl)-[1,1′-biphenyl]-3-yl)-3-methyl-2-(((((S)-oxetan-2-yl)methyl)amino)methyl)pyrrolo[2,1-f][1,2,4]triazin-4(3H)-one;
6-(2,2′-dichloro-3′-(6-methoxy-5-(((((R)-5-oxopyrrolidin-2-yl)methyl)amino)methyl)pyridin-2-yl)-[1,1′-biphenyl]-3-yl)-3-methyl-2-(((((S)-oxetan-2-yl)methyl)amino)methyl)pyrrolo[2,1-f][1,2,4]triazin-4(3H)-one;
6-(2,2′-dichloro-3′-(6-methoxy-5-(((((S)-5-oxopyrrolidin-2-yl)methyl)amino)methyl)pyridin-2-yl)-[1,1′-biphenyl]-3-yl)-3-methyl-2-(((((R)-oxetan-2-yl)methyl)amino)methyl)pyrrolo[2,1-f][1,2,4]triazin-4(3H)-one;
6-(2,2′-dichloro-3′-(6-methoxy-5-(((((R)-5-oxopyrrolidin-2-yl)methyl)amino)methyl)pyridin-2-yl)-[1,1′-biphenyl]-3-yl)-3-methyl-2-(((((R)-oxetan-2-yl)methyl)amino)methyl)pyrrolo[2,1-f][1,2,4]triazin-4(3H)-one;
(S)-6-(2,2′-dichloro-3′-(6-methoxy-5-((((5-oxopyrrolidin-2-yl)methyl)amino)methyl)pyridin-2-yl)-[1,1′-biphenyl]-3-yl)-2-(((3-hydroxybicyclo[1.1.1]pentan-1-yl)amino)methyl)-3-methylpyrrolo[2,1-f][1,2,4]triazin-4(3H)-one;
(R)-6-(2,2′-dichloro-3′-(6-methoxy-5-((((5-oxopyrrolidin-2-yl)methyl)amino)methyl)pyridin-2-yl)-[1,1′-biphenyl]-3-yl)-2-(((3-hydroxybicyclo[1.1.1]pentan-1-yl)amino)methyl)-3-methylpyrrolo[2,1-f][1,2,4]triazin-4(3H)-one;
6-(2,2′-dichloro-3′-(6-methoxy-5-(((((S)-5-oxopyrrolidin-2-yl)methyl)amino)methyl)pyridin-2-yl)-[1,1′-biphenyl]-3-yl)-3-methyl-2-(((((S)-tetrahydrofuran-2-yl)methyl)amino)methyl)pyrrolo[2,1-f][1,2,4]triazin-4(3H)-one;
6-(2,2′-dichloro-3′-(6-methoxy-5-(((((R)-5-oxopyrrolidin-2-yl)methyl)amino)methyl)pyridin-2-yl)-[1,1′-biphenyl]-3-yl)-3-methyl-2-(((((S)-tetrahydrofuran-2-yl)methyl)amino)methyl)pyrrolo[2,1-f][1,2,4]triazin-4(3H)-one;
6-(2,2′-dichloro-3′-(6-methoxy-5-(((((S)-5-oxopyrrolidin-2-yl)methyl)amino)methyl)pyridin-2-yl)-[1,1′-biphenyl]-3-yl)-3-methyl-2-(((((R)-tetrahydrofuran-2-yl)methyl)amino)methyl)pyrrolo[2,1-f][1,2,4]triazin-4(3H)-one;
6-(2,2′-dichloro-3′-(6-methoxy-5-(((((R)-5-oxopyrrolidin-2-yl)methyl)amino)methyl)pyridin-2-yl)-[1,1′-biphenyl]-3-yl)-3-methyl-2-(((((R)-tetrahydrofuran-2-yl)methyl)amino)methyl)pyrrolo[2,1-f][1,2,4]triazin-4(3H)-one;
6-(2,2′-dichloro-3′-(6-methoxy-5-(((((S)-5-oxopyrrolidin-2-yl)methyl)amino)methyl)pyridin-2-yl)-[1,1′-biphenyl]-3-yl)-2-((((S)-2-methoxypropyl)amino)methyl)-3-methylpyrrolo[2,1-f][1,2,4]triazin-4(3H)-one;
6-(2,2′-dichloro-3′-(6-methoxy-5-(((((R)-5-oxopyrrolidin-2-yl)methyl)amino)methyl)pyridin-2-yl)-[1,1′-biphenyl]-3-yl)-2-((((S)-2-methoxypropyl)amino)methyl)-3-methylpyrrolo[2,1-f][1,2,4]triazin-4(3H)-one;
6-(2,2′-dichloro-3′-(6-methoxy-5-(((((S)-5-oxopyrrolidin-2-yl)methyl)amino)methyl)pyridin-2-yl)-[1,1′-biphenyl]-3-yl)-2-((((R)-2-methoxypropyl)amino)methyl)-3-methylpyrrolo[2,1-f][1,2,4]triazin-4(3H)-one;
6-(2,2′-dichloro-3′-(6-methoxy-5-(((((R)-5-oxopyrrolidin-2-yl)methyl)amino)methyl)pyridin-2-yl)-[1,1′-biphenyl]-3-yl)-2-((((R)-2-methoxypropyl)amino)methyl)-3-methylpyrrolo[2,1-f][1,2,4]triazin-4(3H)-one;
6-(2,2′-dichloro-3′-(6-methoxy-5-(((((S)-5-oxopyrrolidin-2-yl)methyl)amino)methyl)pyridin-2-yl)-[1,1′-biphenyl]-3-yl)-3-methyl-2-(((((S)-5-oxopyrrolidin-3-yl)methyl)amino)methyl)pyrrolo[2,1-f][1,2,4]triazin-4(3H)-one;
6-(2,2′-dichloro-3′-(6-methoxy-5-(((((R)-5-oxopyrrolidin-2-yl)methyl)amino)methyl)pyridin-2-yl)-[1,1′-biphenyl]-3-yl)-3-methyl-2-(((((S)-5-oxopyrrolidin-3-yl)methyl)amino)methyl)pyrrolo[2,1-f][1,2,4]triazin-4(3H)-one;
6-(2,2′-dichloro-3′-(6-methoxy-5-(((((S)-5-oxopyrrolidin-2-yl)methyl)amino)methyl)pyridin-2-yl)-[1,1′-biphenyl]-3-yl)-3-methyl-2-(((((R)-5-oxopyrrolidin-3-yl)methyl)amino)methyl)pyrrolo[2,1-f][1,2,4]triazin-4(3H)-one;
6-(2,2′-dichloro-3′-(6-methoxy-5-(((((R)-5-oxopyrrolidin-2-yl)methyl)amino)methyl)pyridin-2-yl)-[1,1′-biphenyl]-3-yl)-3-methyl-2-(((((R)-5-oxopyrrolidin-3-yl)methyl)amino)methyl)pyrrolo[2,1-f][1,2,4]triazin-4(3H)-one;
(S)-6-(2,2′-dichloro-3′-(6-methoxy-5-((((5-oxopyrrolidin-2-yl)methyl)amino)methyl)pyridin-2-yl)-[1,1′-biphenyl]-3-yl)-2-((isopropylamino)methyl)-3-methylpyrrolo[2,1-f][1,2,4]triazin-4(3H)-one;
(R)-6-(2,2′-dichloro-3′-(6-methoxy-5-((((5-oxopyrrolidin-2-yl)methyl)amino)methyl)pyridin-2-yl)-[1,1′-biphenyl]-3-yl)-2-((isopropylamino)methyl)-3-methylpyrrolo[2,1-f][1,2,4]triazin-4(3H)-one;
(S)-6-(2,2′-dichloro-3′-(4-chloro-6-methoxy-5-((((5-oxopyrrolidin-2-yl)methyl)amino)methyl)pyridin-2-yl)-[1,1′-biphenyl]-3-yl)-2-(((2-hydroxy-2-methylpropyl)amino)methyl)-3-methylpyrrolo[2,1-f][1,2,4]triazin-4(3H)-one;
(R)-6-(2,2′-dichloro-3′-(4-chloro-6-methoxy-5-((((5-oxopyrrolidin-2-yl)methyl)amino)methyl)pyridin-2-yl)-[1,1′-biphenyl]-3-yl)-2-(((2-hydroxy-2-methylpropyl)amino)methyl)-3-methylpyrrolo[2,1-f][1,2,4]triazin-4(3H)-one;
(S)-2-((tert-butylamino)methyl)-6-(2,2′-dichloro-3′-(6-methoxy-5-((((5-oxopyrrolidin-2-yl)methyl)amino)methyl)pyridin-2-yl)-[1,1′-biphenyl]-3-yl)-3-methylpyrrolo[2,1-f][1,2,4]triazin-4(3H)-one;
(R)-2-((tert-butylamino)methyl)-6-(2,2′-dichloro-3′-(6-methoxy-5-((((5-oxopyrrolidin-2-yl)methyl)amino)methyl)pyridin-2-yl)-[1,1′-biphenyl]-3-yl)-3-methylpyrrolo[2,1-f][1,2,4]triazin-4(3H)-one;
6-(2,2′-dichloro-3′-(4-fluoro-6-methoxy-5-(((((S)-5-oxopyrrolidin-2-yl)methyl)amino)methyl)pyridin-2-yl)-[1,1′-biphenyl]-3-yl)-2-((((1r,3r)-3-hydroxy-3-methylcyclobutyl)amino)methyl)-3-methylpyrrolo[2,1-f][1,2,4]triazin-4(3H)-one;
6-(2,2′-dichloro-3′-(4-fluoro-6-methoxy-5-(((((R)-5-oxopyrrolidin-2-yl)methyl)amino)methyl)pyridin-2-yl)-[1,1′-biphenyl]-3-yl)-2-((((1r,3r)-3-hydroxy-3-methylcyclobutyl)amino)methyl)-3-methylpyrrolo[2,1-f][1,2,4]triazin-4(3H)-one;
6-(2,2′-dichloro-3′-(4-fluoro-6-methoxy-5-(((((S)-5-oxopyrrolidin-2-yl)methyl)amino)methyl)pyridin-2-yl)-[1,1′-biphenyl]-3-yl)-2-((((1s,3s)-3-hydroxy-3-methylcyclobutyl)amino)methyl)-3-methylpyrrolo[2,1-f][1,2,4]triazin-4(3H)-one;
6-(2,2′-dichloro-3′-(4-fluoro-6-methoxy-5-(((((R)-5-oxopyrrolidin-2-yl)methyl)amino)methyl)pyridin-2-yl)-[1,1′-biphenyl]-3-yl)-2-((((1s,3s)-3-hydroxy-3-methylcyclobutyl)amino)methyl)-3-methylpyrrolo[2,1-f][1,2,4]triazin-4(3H)-one;
(S)-4-(((6-(2,2′-dichloro-3′-(6-methoxy-5-(((((S)-5-oxopyrrolidin-2-yl)methyl)amino)methyl)pyridin-2-yl)-[1,1′-biphenyl]-3-yl)-3-methyl-4-oxo-3,4-dihydropyrrolo[2,1-f][1,2,4]triazin-2-yl)methyl)amino)-3-hydroxy-N,N-dimethylbutanamide;
(R)-4-(((6-(2,2′-dichloro-3′-(6-methoxy-5-(((((S)-5-oxopyrrolidin-2-yl)methyl)amino)methyl)pyridin-2-yl)-[1,1′-biphenyl]-3-yl)-3-methyl-4-oxo-3,4-dihydropyrrolo[2,1-f][1,2,4]triazin-2-yl)methyl)amino)-3-hydroxy-N,N-dimethylbutanamide;
(S)-4-(((6-(2,2′-dichloro-3′-(6-methoxy-5-(((((R)-5-oxopyrrolidin-2-yl)methyl)amino)methyl)pyridin-2-yl)-[1,1′-biphenyl]-3-yl)-3-methyl-4-oxo-3,4-dihydropyrrolo[2,1-f][1,2,4]triazin-2-yl)methyl)amino)-3-hydroxy-N,N-dimethylbutanamide;
(R)-4-(((6-(2,2′-dichloro-3′-(6-methoxy-5-(((((R)-5-oxopyrrolidin-2-yl)methyl)amino)methyl)pyridin-2-yl)-[1,1′-biphenyl]-3-yl)-3-methyl-4-oxo-3,4-dihydropyrrolo[2,1-f][1,2,4]triazin-2-yl)methyl)amino)-3-hydroxy-N,N-dimethylbutanamide;
methyl (S)-((6-(2,2′-dichloro-3′-(6-methoxy-5-((((5-oxopyrrolidin-2-yl)methyl)amino)methyl)pyridin-2-yl)-[1,1′-biphenyl]-3-yl)-3-methyl-4-oxo-3,4-dihydropyrrolo[2,1-f][1,2,4]triazin-2-yl)methyl)glycinate;
methyl (R)-((6-(2,2′-dichloro-3′-(6-methoxy-5-((((5-oxopyrrolidin-2-yl)methyl)amino)methyl)pyridin-2-yl)-[1,1′-biphenyl]-3-yl)-3-methyl-4-oxo-3,4-dihydropyrrolo[2,1-f][1,2,4]triazin-2-yl)methyl)glycinate;
2-(2,2′-dichloro-3′-(6-methoxy-5-(((((S)-5-oxopyrrolidin-2-yl)methyl)amino)methyl)pyridin-2-yl)-[1,1′-biphenyl]-3-yl)-6-(((((S)-5-oxopyrrolidin-2-yl)methyl)amino)methyl)isoindolin-1-one;
2-(2,2′-dichloro-3′-(6-methoxy-5-(((((R)-5-oxopyrrolidin-2-yl)methyl)amino)methyl)pyridin-2-yl)-[1,1′-biphenyl]-3-yl)-6-(((((S)-5-oxopyrrolidin-2-yl)methyl)amino)methyl)isoindolin-1-one;
2-(2,2′-dichloro-3′-(6-methoxy-5-(((((S)-5-oxopyrrolidin-2-yl)methyl)amino)methyl)pyridin-2-yl)-[1,1′-biphenyl]-3-yl)-6-(((((R)-5-oxopyrrolidin-2-yl)methyl)amino)methyl)isoindolin-1-one;
2-(2,2′-dichloro-3′-(6-methoxy-5-(((((R)-5-oxopyrrolidin-2-yl)methyl)amino)methyl)pyridin-2-yl)-[1,1′-biphenyl]-3-yl)-6-(((((R)-5-oxopyrrolidin-2-yl)methyl)amino)methyl)isoindolin-1-one;
(S)-5-((((6-(2,2′-dichloro-3′-(2-(2-(1-hydroxycyclobutyl)ethyl)-8-methoxy-1,2,3,4-tetrahydroisoquinolin-6-yl)-[1,1′-biphenyl]-3-yl)-2-methoxypyridin-3-yl)methyl)amino)methyl)pyrrolidin-2-one;
(R)-5-((((6-(2,2′-dichloro-3′-(2-(2-(1-hydroxycyclobutyl)ethyl)-8-methoxy-1,2,3,4-tetrahydroisoquinolin-6-yl)-[1,1′-biphenyl]-3-yl)-2-methoxypyridin-3-yl)methyl)amino)methyl)pyrrolidin-2-one;
(S)-5-((((6-(2,2′-dichloro-3′-(2-(3-hydroxy-3-methylbutyl)-8-methoxy-1,2,3,4-tetrahydroisoquinolin-6-yl)-[1,1′-biphenyl]-3-yl)-2-methoxypyridin-3-yl)methyl)amino)methyl)pyrrolidin-2-one;
(R)-5-((((6-(2,2′-dichloro-3′-(2-(3-hydroxy-3-methylbutyl)-8-methoxy-1,2,3,4-tetrahydroisoquinolin-6-yl)-[1,1′-biphenyl]-3-yl)-2-methoxypyridin-3-yl)methyl)amino)methyl)pyrrolidin-2-one;
6-(2,2′-dichloro-3′-(6-methoxy-5-(((((S)-5-oxopyrrolidin-2-yl)methyl)amino)methyl)pyridin-2-yl)-[1,1′-biphenyl]-3-yl)-2-((((1r,4r)-4-hydroxycyclohexyl)amino)methyl)-3-methylpyrrolo[2,1-f][1,2,4]triazin-4(3H)-one;
6-(2,2′-dichloro-3′-(6-methoxy-5-(((((S)-5-oxopyrrolidin-2-yl)methyl)amino)methyl)pyridin-2-yl)-[1,1′-biphenyl]-3-yl)-2-((((1s,4s)-4-hydroxycyclohexyl)amino)methyl)-3-methylpyrrolo[2,1-f][1,2,4]triazin-4(3H)-one;
6-(2,2′-dichloro-3′-(6-methoxy-5-(((((R)-5-oxopyrrolidin-2-yl)methyl)amino)methyl)pyridin-2-yl)-[1,1′-biphenyl]-3-yl)-2-((((1r,4r)-4-hydroxycyclohexyl)amino)methyl)-3-methylpyrrolo[2,1-f][1,2,4]triazin-4(3H)-one;
6-(2,2′-dichloro-3′-(6-methoxy-5-(((((R)-5-oxopyrrolidin-2-yl)methyl)amino)methyl)pyridin-2-yl)-[1,1′-biphenyl]-3-yl)-2-((((1s,4s)-4-hydroxycyclohexyl)amino)methyl)-3-methylpyrrolo[2,1-f][1,2,4]triazin-4(3H)-one;
(S)-((6-(2,2′-dichloro-3′-(6-methoxy-5-((((5-oxopyrrolidin-2-yl)methyl)amino)methyl)pyridin-2-yl)-[1,1′-biphenyl]-3-yl)-3-methyl-4-oxo-3,4-dihydropyrrolo[2,1-f][1,2,4]triazin-2-yl)methyl)glycine;
(R)-((6-(2,2′-dichloro-3′-(6-methoxy-5-((((5-oxopyrrolidin-2-yl)methyl)amino)methyl)pyridin-2-yl)-[1,1′-biphenyl]-3-yl)-3-methyl-4-oxo-3,4-dihydropyrrolo[2,1-f][1,2,4]triazin-2-yl)methyl)glycine;
(5S)-5-((((6-(2,2′-dichloro-3′-(2-((2S)-hydroxy-3-methylbutyl)-8-methoxy-1,2,3,4-tetrahydroisoquinolin-6-yl)-[1,1′-biphenyl]-3-yl)-2-methoxypyridin-3-yl)methyl)amino)methyl)pyrrolidin-2-one;
(5R)-5-((((6-(2,2′-dichloro-3′-(2-((2S)-hydroxy-3-methylbutyl)-8-methoxy-1,2,3,4-tetrahydroisoquinolin-6-yl)-[1,1′-biphenyl]-3-yl)-2-methoxypyridin-3-yl)methyl)amino)methyl)pyrrolidin-2-one;
(5S)-5-((((6-(2,2′-dichloro-3′-(2-((2R)-hydroxy-3-methylbutyl)-8-methoxy-1,2,3,4-tetrahydroisoquinolin-6-yl)-[1,1′-biphenyl]-3-yl)-2-methoxypyridin-3-yl)methyl)amino)methyl)pyrrolidin-2-one;
(5R)-5-((((6-(2,2′-dichloro-3′-(2-((2R)-hydroxy-3-methylbutyl)-8-methoxy-1,2,3,4-tetrahydroisoquinolin-6-yl)-[1,1′-biphenyl]-3-yl)-2-methoxypyridin-3-yl)methyl)amino)methyl)pyrrolidin-2-one;
methyl (S)-4-(((6-(2,2′-dichloro-3′-(6-methoxy-5-(((((S)-5-oxopyrrolidin-2-yl)methyl)amino)methyl)pyridin-2-yl)-[1,1′-biphenyl]-3-yl)-3-methyl-4-oxo-3,4-dihydropyrrolo[2,1-f][1,2,4]triazin-2-yl)methyl)amino)-3-hydroxybutanoate;
methyl (R)-4-(((6-(2,2′-dichloro-3′-(6-methoxy-5-(((((S)-5-oxopyrrolidin-2-yl)methyl)amino)methyl)pyridin-2-yl)-[1,1′-biphenyl]-3-yl)-3-methyl-4-oxo-3,4-dihydropyrrolo[2,1-f][1,2,4]triazin-2-yl)methyl)amino)-3-hydroxybutanoate;
methyl (S)-4-(((6-(2,2′-dichloro-3′-(6-methoxy-5-(((((R)-5-oxopyrrolidin-2-yl)methyl)amino)methyl)pyridin-2-yl)-[1,1′-biphenyl]-3-yl)-3-methyl-4-oxo-3,4-dihydropyrrolo[2,1-f][1,2,4]triazin-2-yl)methyl)amino)-3-hydroxybutanoate;
methyl (R)-4-(((6-(2,2′-dichloro-3′-(6-methoxy-5-(((((R)-5-oxopyrrolidin-2-yl)methyl)amino)methyl)pyridin-2-yl)-[1,1′-biphenyl]-3-yl)-3-methyl-4-oxo-3,4-dihydropyrrolo[2,1-f][1,2,4]triazin-2-yl)methyl)amino)-3-hydroxybutanoate;
(S)-6-(2,2′-dichloro-3′-(4-chloro-6-methoxy-5-((((5-oxopyrrolidin-2-yl)methyl)amino)methyl)pyridin-2-yl)-[1,1′-biphenyl]-3-yl)-2-(((3-fluoropropyl)amino)methyl)-3-methylpyrrolo[2,1-f][1,2,4]triazin-4(3H)-one;
(R)-6-(2,2′-dichloro-3′-(4-chloro-6-methoxy-5-((((5-oxopyrrolidin-2-yl)methyl)amino)methyl)pyridin-2-yl)-[1,1′-biphenyl]-3-yl)-2-(((3-fluoropropyl)amino)methyl)-3-methylpyrrolo[2,1-f][1,2,4]triazin-4(3H)-one;
(S)-6-(2,2′-dichloro-3′-(4-chloro-6-methoxy-5-((((5-oxopyrrolidin-2-yl)methyl)amino)methyl)pyridin-2-yl)-[1,1′-biphenyl]-3-yl)-2-(((2-hydroxyethyl)amino)methyl)-3-methylpyrrolo[2,1-f][1,2,4]triazin-4(3H)-one;
(R)-6-(2,2′-dichloro-3′-(4-chloro-6-methoxy-5-((((5-oxopyrrolidin-2-yl)methyl)amino)methyl)pyridin-2-yl)-[1,1′-biphenyl]-3-yl)-2-(((2-hydroxyethyl)amino)methyl)-3-methylpyrrolo[2,1-f][1,2,4]triazin-4(3H)-one;
(S)-6-(2,2′-dichloro-3′-(4-chloro-6-methoxy-5-((((5-oxopyrrolidin-2-yl)methyl)amino)methyl)pyridin-2-yl)-[1,1′-biphenyl]-3-yl)-2-((3-hydroxy-3-methylazetidin-1-yl)methyl)-3-methylpyrrolo[2,1-f][1,2,4]triazin-4(3H)-one;
(R)-6-(2,2′-dichloro-3′-(4-chloro-6-methoxy-5-((((5-oxopyrrolidin-2-yl)methyl)amino)methyl)pyridin-2-yl)-[1,1′-biphenyl]-3-yl)-2-((3-hydroxy-3-methylazetidin-1-yl)methyl)-3-methylpyrrolo[2,1-f][1,2,4]triazin-4(3H)-one;
(S)-4-(((6-(2,2′-dichloro-3′-(6-methoxy-5-(((((S)-5-oxopyrrolidin-2-yl)methyl)amino)methyl)pyridin-2-yl)-[1,1′-biphenyl]-3-yl)-3-methyl-4-oxo-3,4-dihydropyrrolo[2,1-f][1,2,4]triazin-2-yl)methyl)amino)-3-hydroxybutanoic acid;
(R)-4-(((6-(2,2′-dichloro-3′-(6-methoxy-5-(((((S)-5-oxopyrrolidin-2-yl)methyl)amino)methyl)pyridin-2-yl)-[1,1′-biphenyl]-3-yl)-3-methyl-4-oxo-3,4-dihydropyrrolo[2,1-f][1,2,4]triazin-2-yl)methyl)amino)-3-hydroxybutanoic acid;
(S)-4-(((6-(2,2′-dichloro-3′-(6-methoxy-5-(((((R)-5-oxopyrrolidin-2-yl)methyl)amino)methyl)pyridin-2-yl)-[1,1′-biphenyl]-3-yl)-3-methyl-4-oxo-3,4-dihydropyrrolo[2,1-f][1,2,4]triazin-2-yl)methyl)amino)-3-hydroxybutanoic acid;
(R)-4-(((6-(2,2′-dichloro-3′-(6-methoxy-5-(((((R)-5-oxopyrrolidin-2-yl)methyl)amino)methyl)pyridin-2-yl)-[1,1′-biphenyl]-3-yl)-3-methyl-4-oxo-3,4-dihydropyrrolo[2,1-f][1,2,4]triazin-2-yl)methyl)amino)-3-hydroxybutanoic acid;
(S)-1-((6-(2,2′-dichloro-3′-(6-methoxy-5-(((((S)-5-oxopyrrolidin-2-yl)methyl)amino)methyl)pyridin-2-yl)-[1,1′-biphenyl]-3-yl)-2-methoxypyridin-3-yl)methyl)piperidine-2-carboxylic acid;
(R)-1-((6-(2,2′-dichloro-3′-(6-methoxy-5-(((((S)-5-oxopyrrolidin-2-yl)methyl)amino)methyl)pyridin-2-yl)-[1,1′-biphenyl]-3-yl)-2-methoxypyridin-3-yl)methyl)piperidine-2-carboxylic acid;
(S)-1-((6-(2,2′-dichloro-3′-(6-methoxy-5-(((((R)-5-oxopyrrolidin-2-yl)methyl)amino)methyl)pyridin-2-yl)-[1,1′-biphenyl]-3-yl)-2-methoxypyridin-3-yl)methyl)piperidine-2-carboxylic acid;
(R)-1-((6-(2,2′-dichloro-3′-(6-methoxy-5-(((((R)-5-oxopyrrolidin-2-yl)methyl)amino)methyl)pyridin-2-yl)-[1,1′-biphenyl]-3-yl)-2-methoxypyridin-3-yl)methyl)piperidine-2-carboxylic acid;
ethyl (S)-3-(((6-(2,2′-dichloro-3′-(6-methoxy-5-((((5-oxopyrrolidin-2-yl)methyl)amino)methyl)pyridin-2-yl)-[1,1′-biphenyl]-3-yl)-3-methyl-4-oxo-3,4-dihydropyrrolo[2,1-f][1,2,4]triazin-2-yl)methyl)amino)-2,2-dimethylpropanoate;
methyl (R)-3-(((6-(2,2′-dichloro-3′-(6-methoxy-5-((((5-oxopyrrolidin-2-yl)methyl)amino)methyl)pyridin-2-yl)-[1,1′-biphenyl]-3-yl)-3-methyl-4-oxo-3,4-dihydropyrrolo[2,1-f][1,2,4]triazin-2-yl)methyl)amino)-2,2-dimethylpropanoate;
methyl (S)-1-((6-(2,2′-dichloro-3′-(6-methoxy-5-((((5-oxopyrrolidin-2-yl)methyl)amino)methyl)pyridin-2-yl)-[1,1′-biphenyl]-3-yl)-3-methyl-4-oxo-3,4-dihydropyrrolo[2,1-f][1,2,4]triazin-2-yl)methyl)azetidine-3-carboxylate;
methyl (R)-1-((6-(2,2′-dichloro-3′-(6-methoxy-5-((((5-oxopyrrolidin-2-yl)methyl)amino)methyl)pyridin-2-yl)-[1,1′-biphenyl]-3-yl)-3-methyl-4-oxo-3,4-dihydropyrrolo[2,1-f][1,2,4]triazin-2-yl)methyl)azetidine-3-carboxylate;
(S)-5-((((6-(2,2′-dichloro-3′-(8-methoxy-2-(2-(piperidin-4-yl)ethyl)-1,2,3,4-tetrahydroisoquinolin-6-yl)-[1,1′-biphenyl]-3-yl)-2-methoxypyridin-3-yl)methyl)amino)methyl)pyrrolidin-2-one;
(R)-5-((((6-(2,2′-dichloro-3′-(8-methoxy-2-(2-(piperidin-4-yl)ethyl)-1,2,3,4-tetrahydroisoquinolin-6-yl)-[1,1′-biphenyl]-3-yl)-2-methoxypyridin-3-yl)methyl)amino)methyl)pyrrolidin-2-one;
(S)-5-((((6-(3′-(2-((1-acetylpiperidin-4-yl)methyl)-8-methoxy-1,2,3,4-tetrahydroisoquinolin-6-yl)-2,2′-dichloro-[1,1′-biphenyl]-3-yl)-2-methoxypyridin-3-yl)methyl)amino)methyl)pyrrolidin-2-one;
(R)-5-((((6-(3′-(2-((1-acetylpiperidin-4-yl)methyl)-8-methoxy-1,2,3,4-tetrahydroisoquinolin-6-yl)-2,2′-dichloro-[1,1′-biphenyl]-3-yl)-2-methoxypyridin-3-yl)methyl)amino)methyl)pyrrolidin-2-one;
(S)-6-(2,2′-dichloro-3′-(4-fluoro-6-methoxy-5-((((5-oxopyrrolidin-2-yl)methyl)amino)methyl)pyridin-2-yl)-[1,1′-biphenyl]-3-yl)-2-((3-hydroxy-3-methylazetidin-1-yl)methyl)-3-methylpyrrolo[2,1-f][1,2,4]triazin-4(3H)-one;
(R)-6-(2,2′-dichloro-3′-(4-fluoro-6-methoxy-5-((((5-oxopyrrolidin-2-yl)methyl)amino)methyl)pyridin-2-yl)-[1,1′-biphenyl]-3-yl)-2-((3-hydroxy-3-methylazetidin-1-yl)methyl)-3-methylpyrrolo[2,1-f][1,2,4]triazin-4(3H)-one;
(S)-6-(2,2′-dichloro-3′-(4-fluoro-6-methoxy-5-((((5-oxopyrrolidin-2-yl)methyl)amino)methyl)pyridin-2-yl)-[1,1′-biphenyl]-3-yl)-2-(((2-hydroxyethyl)amino)methyl)-3-methylpyrrolo[2,1-f][1,2,4]triazin-4(3H)-one;
(R)-6-(2,2′-dichloro-3′-(4-fluoro-6-methoxy-5-((((5-oxopyrrolidin-2-yl)methyl)amino)methyl)pyridin-2-yl)-[1,1′-biphenyl]-3-yl)-2-(((2-hydroxyethyl)amino)methyl)-3-methylpyrrolo[2,1-f][1,2,4]triazin-4(3H)-one;
(S)-1-((6-(2,2′-dichloro-3′-(6-methoxy-5-(((((S)-5-oxopyrrolidin-2-yl)methyl)amino)methyl)pyridin-2-yl)-[1,1′-biphenyl]-3-yl)-3-methyl-4-oxo-3,4-dihydropyrrolo[2,1-f][1,2,4]triazin-2-yl)methyl)pyrrolidine-3-carboxylic acid;
(R)-1-((6-(2,2′-dichloro-3′-(6-methoxy-5-(((((S)-5-oxopyrrolidin-2-yl)methyl)amino)methyl)pyridin-2-yl)-[1,1′-biphenyl]-3-yl)-3-methyl-4-oxo-3,4-dihydropyrrolo[2,1-f][1,2,4]triazin-2-yl)methyl)pyrrolidine-3-carboxylic acid;
(S)-1-((6-(2,2′-dichloro-3′-(6-methoxy-5-(((((R)-5-oxopyrrolidin-2-yl)methyl)amino)methyl)pyridin-2-yl)-[1,1′-biphenyl]-3-yl)-3-methyl-4-oxo-3,4-dihydropyrrolo[2,1-f][1,2,4]triazin-2-yl)methyl)pyrrolidine-3-carboxylic acid;
(R)-1-((6-(2,2′-dichloro-3′-(6-methoxy-5-(((((R)-5-oxopyrrolidin-2-yl)methyl)amino)methyl)pyridin-2-yl)-[1,1′-biphenyl]-3-yl)-3-methyl-4-oxo-3,4-dihydropyrrolo[2,1-f][1,2,4]triazin-2-yl)methyl)pyrrolidine-3-carboxylic acid;
(S)-1-(((6-(2,2′-dichloro-3′-(6-methoxy-5-((((5-oxopyrrolidin-2-yl)methyl)amino)methyl)pyridin-2-yl)-[1,1′-biphenyl]-3-yl)-3-methyl-4-oxo-3,4-dihydropyrrolo[2,1-f][1,2,4]triazin-2-yl)methyl)amino)cyclopropane-1-carboxylic acid;
(R)-1-(((6-(2,2′-dichloro-3′-(6-methoxy-5-((((5-oxopyrrolidin-2-yl)methyl)amino)methyl)pyridin-2-yl)-[1,1′-biphenyl]-3-yl)-3-methyl-4-oxo-3,4-dihydropyrrolo[2,1-f][1,2,4]triazin-2-yl)methyl)amino)cyclopropane-1-carboxylic acid;
(S)-3-(((6-(2,2′-dichloro-3′-(6-methoxy-5-((((5-oxopyrrolidin-2-yl)methyl)amino)methyl)pyridin-2-yl)-[1,1′-biphenyl]-3-yl)-3-methyl-4-oxo-3,4-dihydropyrrolo[2,1-f][1,2,4]triazin-2-yl)methyl)amino)-2,2-dimethylpropanoic acid;
(R)-3-(((6-(2,2′-dichloro-3′-(6-methoxy-5-((((5-oxopyrrolidin-2-yl)methyl)amino)methyl)pyridin-2-yl)-[1,1′-biphenyl]-3-yl)-3-methyl-4-oxo-3,4-dihydropyrrolo[2,1-f][1,2,4]triazin-2-yl)methyl)amino)-2,2-dimethylpropanoic acid;
methyl ((6-(2,2′-dichloro-3′-(6-methoxy-5-(((((S)-5-oxopyrrolidin-2-yl)methyl)amino)methyl)pyridin-2-yl)-[1,1′-biphenyl]-3-yl)-3-methyl-4-oxo-3,4-dihydropyrrolo[2,1-f][1,2,4]triazin-2-yl)methyl)-D-alaninate;
methyl ((6-(2,2′-dichloro-3′-(6-methoxy-5-(((((R)-5-oxopyrrolidin-2-yl)methyl)amino)methyl)pyridin-2-yl)-[1,1′-biphenyl]-3-yl)-3-methyl-4-oxo-3,4-dihydropyrrolo[2,1-f][1,2,4]triazin-2-yl)methyl)-D-alaninate;
methyl ((6-(2,2′-dichloro-3′-(6-methoxy-5-(((((S)-5-oxopyrrolidin-2-yl)methyl)amino)methyl)pyridin-2-yl)-[1,1′-biphenyl]-3-yl)-3-methyl-4-oxo-3,4-dihydropyrrolo[2,1-f][1,2,4]triazin-2-yl)methyl)-L-alaninate;
methyl ((6-(2,2′-dichloro-3′-(6-methoxy-5-(((((R)-5-oxopyrrolidin-2-yl)methyl)amino)methyl)pyridin-2-yl)-[1,1′-biphenyl]-3-yl)-3-methyl-4-oxo-3,4-dihydropyrrolo[2,1-f][1,2,4]triazin-2-yl)methyl)-L-alaninate;
(S)-1-((6-(2,2′-dichloro-3′-(6-methoxy-5-((((5-oxopyrrolidin-2-yl)methyl)amino)methyl)pyridin-2-yl)-[1,1′-biphenyl]-3-yl)-3-methyl-4-oxo-3,4-dihydropyrrolo[2,1-f][1,2,4]triazin-2-yl)methyl)azetidine-3-carboxylic acid;
(R)-1-((6-(2,2′-dichloro-3′-(6-methoxy-5-((((5-oxopyrrolidin-2-yl)methyl)amino)methyl)pyridin-2-yl)-[1,1′-biphenyl]-3-yl)-3-methyl-4-oxo-3,4-dihydropyrrolo[2,1-f][1,2,4]triazin-2-yl)methyl)azetidine-3-carboxylic acid;
(5S)-5-[[[6-[2-chloro-3-[2-chloro-3-[2-[[[(2S)-5-oxopyrrolidin-2-yl]methylamino]methyl]-[1,2,4]triazolo[1,5-a]pyrimidin-6-yl]phenyl]phenyl]-2-methoxy-3-pyridyl]methylamino]methyl]pyrrolidin-2-one;
(5R)-5-[[[6-[2-chloro-3-[2-chloro-3-[2-[[[(2S)-5-oxopyrrolidin-2-yl]methylamino]methyl]-[1,2,4]triazolo[1,5-a]pyrimidin-6-yl]phenyl]phenyl]-2-methoxy-3-pyridyl]methylamino]methyl]pyrrolidin-2-one;
(5S)-5-[[[6-[2-chloro-3-[2-chloro-3-[2-[[[(2R)-5-oxopyrrolidin-2-yl]methylamino]methyl]-[1,2,4]triazolo[1,5-a]pyrimidin-6-yl]phenyl]phenyl]-2-methoxy-3-pyridyl]methylamino]methyl]pyrrolidin-2-one;
(5R)-5-[[[6-[2-chloro-3-[2-chloro-3-[2-[[[(2R)-5-oxopyrrolidin-2-yl]methylamino]methyl]-[1,2,4]triazolo[1,5-a]pyrimidin-6-yl]phenyl]phenyl]-2-methoxy-3-pyridyl]methylamino]methyl]pyrrolidin-2-one;
(2R)-2-[[6-[2-chloro-3-[2-chloro-3-[6-methoxy-5-[[[(2S)-5-oxopyrrolidin-2-yl]methylamino]methyl]-2-pyridyl]phenyl]phenyl]-3-methyl-4-oxo-pyrrolo[2,1-f][1,2,4]triazin-2-yl]methylamino]propanoic acid;
(2R)-2-[[6-[2-chloro-3-[2-chloro-3-[6-methoxy-5-[[[(2R)-5-oxopyrrolidin-2-yl]methylamino]methyl]-2-pyridyl]phenyl]phenyl]-3-methyl-4-oxo-pyrrolo[2,1-f][1,2,4]triazin-2-yl]methylamino]propanoic acid;
(2S)-2-[[6-[2-chloro-3-[2-chloro-3-[6-methoxy-5-[[[(2S)-5-oxopyrrolidin-2-yl]methylamino]methyl]-2-pyridyl]phenyl]phenyl]-3-methyl-4-oxo-pyrrolo[2,1-f][1,2,4]triazin-2-yl]methylamino]propanoic acid;
(2S)-2-[[6-[2-chloro-3-[2-chloro-3-[6-methoxy-5-[[[(2R)-5-oxopyrrolidin-2-yl]methylamino]methyl]-2-pyridyl]phenyl]phenyl]-3-methyl-4-oxo-pyrrolo[2,1-f][1,2,4]triazin-2-yl]methylamino]propanoic acid;
(S)-6-(2,2′-Dichloro-3′-(4-fluoro-6-methoxy-5-((((5-oxopyrrolidin-2-yl)methyl)amino)methyl)pyridin-2-yl)-[1,1′-biphenyl]-3-yl)-2-(((3-fluoropropyl)amino)methyl)-3-methylpyrrolo[2,1-f][1,2,4]triazin-4(3H)-one;
(R)-6-(2,2′-Dichloro-3′-(4-fluoro-6-methoxy-5-((((5-oxopyrrolidin-2-yl)methyl)amino)methyl)pyridin-2-yl)-[1,1′-biphenyl]-3-yl)-2-(((3-fluoropropyl)amino)methyl)-3-methylpyrrolo[2,1-f][1,2,4]triazin-4(3H)-one;
(S)-5-((6-(2,2′-Dichloro-3′-(3-fluoro-6-methoxy-5-(((((S)-5-oxopyrrolidin-2-yl)methyl)amino)methyl)pyridin-2-yl)-[1,1′-biphenyl]-3-yl)-8-methoxy-3,4-dihydroisoquinolin-2(1H)-yl)methyl)pyrrolidin-2-one;
(R)-5-((6-(2,2′-Dichloro-3′-(3-fluoro-6-methoxy-5-(((((S)-5-oxopyrrolidin-2-yl)methyl)amino)methyl)pyridin-2-yl)-[1,1′-biphenyl]-3-yl)-8-methoxy-3,4-dihydroisoquinolin-2(1H)-yl)methyl)pyrrolidin-2-one;
(S)-5-((6-(2,2′-Dichloro-3′-(3-fluoro-6-methoxy-5-(((((R)-5-oxopyrrolidin-2-yl)methyl)amino)methyl)pyridin-2-yl)-[1,1′-biphenyl]-3-yl)-8-methoxy-3,4-dihydroisoquinolin-2(1H)-yl)methyl)pyrrolidin-2-one;
(R)-5-((6-(2,2′-Dichloro-3′-(3-fluoro-6-methoxy-5-(((((R)-5-oxopyrrolidin-2-yl)methyl)amino)methyl)pyridin-2-yl)-[1,1′-biphenyl]-3-yl)-8-methoxy-3,4-dihydroisoquinolin-2(1H)-yl)methyl)pyrrolidin-2-one;
2-(2,2′-Dichloro-3′-(6-methoxy-5-(((((S)-5-oxopyrrolidin-2-yl)methyl)amino)methyl)pyridin-2-yl)-[1,1′-biphenyl]-3-yl)-1-methyl-5-(((((S)-5-oxopyrrolidin-2-yl)methyl)amino)methyl)pyrazolo[1,5-a]pyrimidin-7(1H)-one;
2-(2,2′-Dichloro-3′-(6-methoxy-5-(((((R)-5-oxopyrrolidin-2-yl)methyl)amino)methyl)pyridin-2-yl)-[1,1′-biphenyl]-3-yl)-1-methyl-5-(((((S)-5-oxopyrrolidin-2-yl)methyl)amino)methyl)pyrazolo[1,5-a]pyrimidin-7(1H)-one;
2-(2,2′-Dichloro-3′-(6-methoxy-5-(((((S)-5-oxopyrrolidin-2-yl)methyl)amino)methyl)pyridin-2-yl)-[1,1′-biphenyl]-3-yl)-1-methyl-5-(((((R)-5-oxopyrrolidin-2-yl)methyl)amino)methyl)pyrazolo[1,5-a]pyrimidin-7(1H)-one;
2-(2,2′-Dichloro-3′-(6-methoxy-5-(((((R)-5-oxopyrrolidin-2-yl)methyl)amino)methyl)pyridin-2-yl)-[1,1′-biphenyl]-3-yl)-1-methyl-5-(((((R)-5-oxopyrrolidin-2-yl)methyl)amino)methyl)pyrazolo[1,5-a]pyrimidin-7(1H)-one;
6-(2,2′-Dichloro-3′-(3-fluoro-6-methoxy-5-(((((S)-5-oxopyrrolidin-2-yl)methyl)amino)methyl)pyridin-2-yl)-[1,1′-biphenyl]-3-yl)-2-((((1r,3r)-3-hydroxy-3-methylcyclobutyl)amino)methyl)-3-methylpyrrolo[2,1-f][1,2,4]triazin-4(3H)-one;
6-(2,2′-Dichloro-3′-(3-fluoro-6-methoxy-5-(((((S)-5-oxopyrrolidin-2-yl)methyl)amino)methyl)pyridin-2-yl)-[1,1′-biphenyl]-3-yl)-2-((((1s,3s)-3-hydroxy-3-methylcyclobutyl)amino)methyl)-3-methylpyrrolo[2,1-f][1,2,4]triazin-4(3H)-one;
6-(2,2′-Dichloro-3′-(3-fluoro-6-methoxy-5-(((((R)-5-oxopyrrolidin-2-yl)methyl)amino)methyl)pyridin-2-yl)-[1,1′-biphenyl]-3-yl)-2-((((1r,3r)-3-hydroxy-3-methylcyclobutyl)amino)methyl)-3-methylpyrrolo[2,1-f][1,2,4]triazin-4(3H)-one;
6-(2,2′-Dichloro-3′-(3-fluoro-6-methoxy-5-(((((R)-5-oxopyrrolidin-2-yl)methyl)amino)methyl)pyridin-2-yl)-[1,1′-biphenyl]-3-yl)-2-((((1s,3s)-3-hydroxy-3-methylcyclobutyl)amino)methyl)-3-methylpyrrolo[2,1-f][1,2,4]triazin-4(3H)-one;
(S)-Isopropyl 2-(((6-(2,2′-dichloro-3′-(4-chloro-6-methoxy-5-((((5-oxopyrrolidin-2-yl)methyl)amino)methyl)pyridin-2-yl)-[1,1′-biphenyl]-3-yl)-3-methyl-4-oxo-3,4-dihydropyrrolo[2,1-f][1,2,4]triazin-2-yl)methyl)amino)-2-methylpropanoate;
(R)-Isopropyl 2-(((6-(2,2′-dichloro-3′-(4-chloro-6-methoxy-5-((((5-oxopyrrolidin-2-yl)methyl)amino)methyl)pyridin-2-yl)-[1,1′-biphenyl]-3-yl)-3-methyl-4-oxo-3,4-dihydropyrrolo[2,1-f][1,2,4]triazin-2-yl)methyl)amino)-2-methylpropanoate;
(3S)-1-[[6-[2-Chloro-3-[2-chloro-3-[6-methoxy-5-[[[(2S)-5-oxopyrrolidin-2-yl]methylamino]methyl]-2-pyridyl]phenyl]phenyl]-3-methyl-4-oxo-pyrrolo[2,1-f][1,2,4]triazin-2-yl]methyl]piperidine-3-carboxylic acid;
(3S)-1-[[6-[2-Chloro-3-[2-chloro-3-[6-methoxy-5-[[[(2R)-5-oxopyrrolidin-2-yl]methylamino]methyl]-2-pyridyl]phenyl]phenyl]-3-methyl-4-oxo-pyrrolo[2,1-f][1,2,4]triazin-2-yl]methyl]piperidine-3-carboxylic acid;
(3R)-1-[[6-[2-Chloro-3-[2-chloro-3-[6-methoxy-5-[[[(2S)-5-oxopyrrolidin-2-yl]methylamino]methyl]-2-pyridyl]phenyl]phenyl]-3-methyl-4-oxo-pyrrolo[2,1-f][1,2,4]triazin-2-yl]methyl]piperidine-3-carboxylic acid;
(3R)-1-[[6-[2-Chloro-3-[2-chloro-3-[6-methoxy-5-[[[(2R)-5-oxopyrrolidin-2-yl]methylamino]methyl]-2-pyridyl]phenyl]phenyl]-3-methyl-4-oxo-pyrrolo[2,1-f][1,2,4]triazin-2-yl]methyl]piperidine-3-carboxylic acid;
(S)-2-(2,2′-Dichloro-3′-(6-methoxy-5-((((5-oxopyrrolidin-2-yl)methyl)amino)methyl)pyridin-2-yl)-[1,1′-biphenyl]-3-yl)-5-(((2-hydroxyethyl)amino)methyl)-1-methylpyrazolo[1,5-a]pyrimidin-7(1H)-one;
(R)-2-(2,2′-Dichloro-3′-(6-methoxy-5-((((5-oxopyrrolidin-2-yl)methyl)amino)methyl)pyridin-2-yl)-[1,1′-biphenyl]-3-yl)-5-(((2-hydroxyethyl)amino)methyl)-1-methylpyrazolo[1,5-a]pyrimidin-7(1H)-one;
(5S)-5-[[[6-[3-[3-[2-[[(1-Acetyl-4-piperidyl)amino]methyl]-[1,2,4]triazolo[1,5-a]pyrimidin-6-yl]-2-chloro-phenyl]-2-chloro-phenyl]-2-methoxy-3-pyridyl]methylamino]methyl] pyrrolidin-2-one;
(5R)-5-[[[6-[3-[3-[2-[[(1-Acetyl-4-piperidyl)amino]methyl]-[1,2,4]triazolo[1,5-a]pyrimidin-6-yl]-2-chloro-phenyl]-2-chloro-phenyl]-2-methoxy-3-pyridyl]methylamino]methyl] pyrrolidin-2-one;
3-[[6-[2-Chloro-3-[2-chloro-3-[6-methoxy-5-[[[(2S)-5-oxopyrrolidin-2-yl]methylamino]methyl]-2-pyridyl]phenyl]phenyl]-3-methyl-4-oxo-pyrrolo[2,1-f][1,2,4]triazin-2-yl]methylamino]propanoic acid;
3-[[6-[2-Chloro-3-[2-chloro-3-[6-methoxy-5-[[[(2R)-5-oxopyrrolidin-2-yl]methylamino]methyl]-2-pyridyl]phenyl]phenyl]-3-methyl-4-oxo-pyrrolo[2,1-f][1,2,4]triazin-2-yl]methylamino]propanoic acid;
(S)-2-(2,2′-Dichloro-3′-(6-methoxy-5-((((5-oxopyrrolidin-2-yl)methyl)amino)methyl)pyridin-2-yl)-[1,1′-biphenyl]-3-yl)-5-((6-hydroxy-2-azaspiro[3.3]heptan-2-yl)methyl)-1-methylpyrazolo[1,5-a]pyrimidin-7(1H)-one;
(R)-2-(2,2′-Dichloro-3′-(6-methoxy-5-((((5-oxopyrrolidin-2-yl)methyl)amino)methyl)pyridin-2-yl)-[1,1′-biphenyl]-3-yl)-5-((6-hydroxy-2-azaspiro[3.3]heptan-2-yl)methyl)-1-methylpyrazolo[1,5-a]pyrimidin-7(1H)-one;

19. A pharmaceutical composition comprising at least one compound of claim 1 and at least one pharmaceutically acceptable carrier, wherein the hepatitis virus is at least one of hepatitis B virus (HBV) and hepatitis D virus (HDV), optionally further comprising at least one additional agent that treats, ameliorates, or prevents hepatitis virus infection.

20. (canceled)

21. The pharmaceutical composition of claim 19, wherein the at least one additional agent comprises at least one selected from the group consisting of reverse transcriptase inhibitor; capsid inhibitor; cccDNA formation inhibitor; RNA destabilizer; oligomeric nucleotide targeted against the HBV genome; immunostimulator; and GalNAc-siRNA conjugate targeted against an HBV gene transcript, optionally wherein the immunostimulator is a checkpoint inhibitor.

22-24. (canceled)

25. A method of treating, ameliorating, or preventing hepatitis B virus infection in a subject, the method comprising administering to the subject in need thereof a therapeutically effective amount of the compound of claim 1, or a salt, solvate, prodrug, stereoisomer, tautomer, or any mixtures thereof, optionally wherein the subject is further infected with hepatitis D virus (HDV), optionally wherein the subject is further administered at least one additional agent useful for treating, ameliorating, or preventing the hepatitis virus infection; optionally wherein the subject is a mammal.

26-29. (canceled)

30. The method of claim 25, wherein the at least one additional agent comprises at least one selected from the group consisting of reverse transcriptase inhibitor; capsid inhibitor; cccDNA formation inhibitor; RNA destabilizer; oligomeric nucleotide targeted against the HBV genome; immunostimulator; and GalNAc-siRNA conjugate targeted against an HBV gene transcript, optionally wherein the immunostimulator is a checkpoint inhibitor.

31-32. (canceled)

33. The method of claim 25, wherein the subject is co-administered the at least one compound and the at least one additional agent, optionally wherein the at least one compound and the at least one additional agent are coformulated.

34. (canceled)

35. A method of treating, ameliorating, aid/or preventing cancer in a subject, the method comprising administering to the subject in need thereof a therapeutically effective amount of the compound of claim 1, or a salt, solvate, prodrug, stereoisomer, tautomer, or any mixtures thereof, optionally wherein the compound is administered to the subject in a pharmaceutically acceptable composition, optionally wherein the subject is a mammal.

36. The method of claim 35, wherein the compound is the only anticancer agent administered to the subject or wherein the subject is further administered at least one additional agent or therapy useful for treating, ameliorating, or preventing cancer.

37-38. (canceled)

39. The method of claim 36, wherein the additional anticancer agent or therapy comprises rituxan, doxorubicin, gemcitabine, nivolumab, pembrolizumab, atezolizumab, ipilimumab, chemotherapy, radiation therapy, or resection therapy.

40. (canceled)

41. The method of claim 36, wherein the compound is coformulated or co-administered with the at least one additional agent.

42. The method of claim 35, wherein the cancer is amenable to treatment by inhibiting PD-1, PD-L1 or the PD-1/PD-L1 interaction.

43. The method of claim 35, wherein the cancer is at least one of:
(a) pancreatic cancer, bladder cancer, colorectal cancer, breast cancer, prostate cancer, renal cancer, hepatocellular cancer, lung cancer, ovarian cancer, cervical cancer, gastric cancer, esophageal cancer, head and neck cancer, melanoma, neuroendocrine cancer, CNS cancer, brain cancer, bone cancer, soft tissue sarcoma, non-small cell lung cancer, small-cell lung cancer, or colon cancer;
(b) lymphoma, multiple myeloma, or leukemia;
(c) acute lymphocytic leukemia (ALL), acute myeloid leukemia (AML), chronic lymphocytic leukemia (CLL), small lymphocytic lymphoma (SLL), myelodysplastic syndrome (MDS), myeloproliferative disease (MPD), chronic myeloid leukemia (CML), multiple myeloma (MM), non-Hodgkin's lymphoma (NHL), mantle cell lymphoma (MCL), follicular lymphoma, Waldestrom's macroglobulinemia (WM), T-cell lymphoma, B-cell lymphoma, or diffuse large B-cell lymphoma (DLBCL).

44-47. (canceled)
